(19) United States
(12) Patent Application Publication
(10) Pub. No.: US 2026/0355196 A1
(43) Pub. Date: Nov. 12, 2026

(54) INHIBITORS AND DEGRADERS OF PIP4K PROTEIN

(71) Applicant: Larkspur Biosciences, Inc., Cambridge, MA (US)

(72) Inventors: Andrew GOOD, Westward Ho (GB); Kristin BROWN, Limerick, PA (US); Krista B. GOODMAN, Belmont, MA (US); Guosen YE, Waltham, MA (US); Morgan O'SHEA, Waltham, MA (US)

(21) Appl. No.: 19/482,426

(22) PCT Filed: May 9, 2024
(86) PCT No.: PCT/US2024/028686

§ 371 Date: Nov. 7, 2025

Related U.S. Application Data
(60) Provisional application No. 63/465,458, filed on May 10, 2023.
(60) Provisional application No. 63/611,367, filed on Dec. 18, 2023.
(60) Provisional application No. 63/575,561, filed on Apr. 5, 2024.

(51) Int. Cl. A61K 31/506; A61K 31/505; A61K 31/5355; A61K 31/5377; A61K 39/39; A61P 35/00; C07D 239/42; C07D 401/10; C07D 401/12; C07D 401/14; C07D 403/04; C07D 403/10; C07D 405/10; C07D 413/10; C07D 413/14

(52) U.S. Cl. CPC A61K 31/506; A61K 31/505; A61K 31/5355; A61K 31/5377; A61K 39/39; A61P 35/00; C07D 239/42; C07D 401/10; C07D 401/12; C07D 401/14; C07D 403/04; C07D 403/10; C07D 405/10; C07D 413/10; C07D 413/14

(57) ABSTRACT

Provided are compounds and compositions of Formula (I′): which modulate the level or activity of PIP4K2A, PIP4K2B, or PIP4K2C. Also provided are methods for treating diseases or conditions by modulating (e.g., reducing) the level or activity of PIP4K2A, PIP4K2B, or PIP4K2C, comprising administering the compounds and compositions.

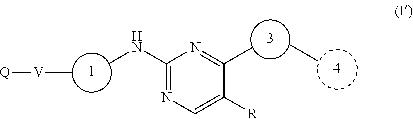

### CROSS-REFERENCE TO RELATED APPLICATIONS

This application claims priority to U.S. Provisional Application No. 63/465,458, filed May 10, 2023; U.S. Provisional Application No. 63/611,367, filed Dec. 18, 2023; and U.S. Provisional Application No. 63/575,561, filed Apr. 5, 2024; the contents of which are hereby incorporated by reference in their entirety for all purposes.

### BACKGROUND

The phosphatidylinositol 5-phosphate 4-kinases (PI5P4Ks), consisting of the three isoforms, PI5P4Kα, β, and γ, are lipid kinases that catalyze phosphorylation of phosphatidylinositol 5-phosphate (PI5P) on its 4-position to form phosphatidylinositol-4,5-bisphosphate (PI-4,5-P2) (Rameh et al., Nature 390(6656):192-196 (1997). In the cellular membrane, PI-4,5-P2 is also produced by another signaling pathway in which phosphatidylinositol 4-phosphate (PI4P) is phosphorylated by phosphatidylinositol 4-phosphate 5-kinases (PI4P5Ks).

Although the majority of PI-4,5-P2 is produced via the PI4P5K pathway, PI5P4Ks have been recognized as key regulators of many cell functions including metabolism, stress response, autophagy, and immunological processes (Hu et al., J. Lipid Res. 59:507-514 (2018); Lamia et al., Mol. Cell. Biol. 24(11):5080-5087 (2004); Shim et al., Proc. Natl. Acad. Sci. U.S.A 113(27):7596-7601 (2016); Al-Ramahi et al., eLife 6:e29123 (2017); Lundquist et al., Mol. Cell 70(3):531-543 (2018); Bulley et al., Proc. Natl. Acad. Sci. U.S.A 113(38):10571-10576 (2016); Keune et al., Adv. Biol. Regul. 53(2):179-189 (2013)). PIP4Ks have distinct catalytic and non-catalytic functions in controlling cellular metabolism and suppress PIP5K activity and insulin-stimulated production of PI(3,4,5)P3 (Wang et al., Cell Rep. 27:1991-2001 (2019)).

Dysregulation of the PI5P4K signaling pathway has been further linked to diseases such as diabetes, neurodegenerative disorders, and cancers (Lamia et al., Mol. Cell. Biol. 24(11): 5080-5087 (2004); Al-Ramahi et al., eLife 6:e29123 (2017); Jude et al., Oncogene 34(10):1253-1262 (2015); Luoh et al., Oncogene 23:1354-1363 (2004); Emerling et al., Cell 155(4):844-857 (2013). Analysis of PI(4,5)P2 levels in cells with single or double knockdown of PIP4K isoforms revealed an additive effect among all three isoforms that does not correlate with their relative catalytic activities. Double knockdown of the most active isoforms, PIP4KA/B, failed to phenocopy the triple knockdown, suggesting that catalytic activity is not the most important factor in regulating PI(4,5)P2 levels (Wang et al., Cell Rep. 27:1991-2001 (2019), and suggesting a unique role for PIP4KC.

These findings indicate that the inhibition of PI5P4K kinase activity and/or degradation of PI4P4K might have therapeutic potential across various diseases.

### SUMMARY OF THE INVENTION

In one aspect, provided is a compound of Formula (I′):

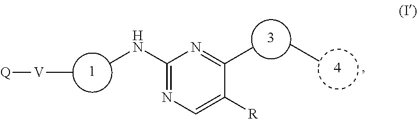

or a tautomer or stereoisomer thereof, or a pharmaceutically acceptable salt of any of the foregoing, wherein Q, V, R, Ring 1, Ring 3, and Ring 4 are as detailed herein.

In one aspect, provided is a compound of Formula (I):

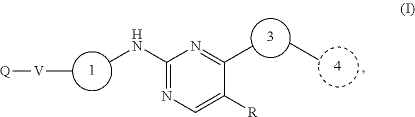

or a tautomer or stereoisomer thereof, or a pharmaceutically acceptable salt of any of the foregoing, wherein Q, V, R, Ring 1, Ring 3, and Ring 4 are as detailed herein. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I), Q is Ring 5, which is as detailed herein. In some embodiments, provided is a compound of Formula (II):

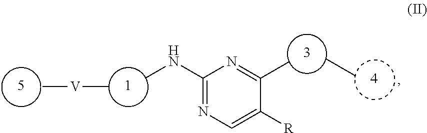

or a tautomer or stereoisomer thereof, or a pharmaceutically acceptable salt of any of the foregoing, wherein V, R, Ring 1, Ring 3, Ring 4, and Ring 5 are as detailed herein. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I), Q is R^{Q}, which is as detailed herein. In some embodiments, provided is a compound of Formula (III):

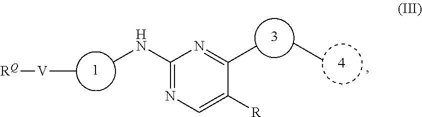

or a tautomer or stereoisomer thereof, or a pharmaceutically acceptable salt of any of the foregoing, wherein V, R, Ring 1, Ring 3, Ring 4, and R^{Q} are as detailed herein. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I), Q is of Formula (i)

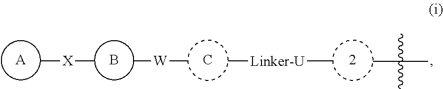

wherein Ring A, X, Ring B, W, Ring C, Linker, U, and Ring 2 are as detailed herein. In some embodiments, provided is a compound of Formula (IV):

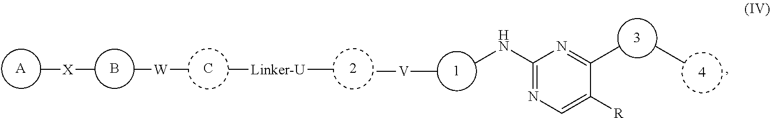

or a tautomer or stereoisomer thereof, or a pharmaceutically acceptable salt of any of the foregoing, wherein Ring A, X, Ring B, W, Ring C, Linker, U, Ring 2, V, Ring 1, Ring 3, and Ring 4 are as detailed herein. In some variations, the embodiments provided herein also apply to a compound of Formula (I′), or a stereoisomer or tautomer thereof, or a pharmaceutically acceptable salt of any of the foregoing, or any variation or embodiment thereof.

In another aspect, provided is a pharmaceutical composition comprising a compound of Formula (I), (II), (III), or (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier. In some embodiments, provided is a pharmaceutical composition comprising a therapeutically effective amount of a compound of Formula (I), (II), (III), or (IV), or any related formula such as Formula (I-a), (I-b), (I-c), (I-d), (I-e), (I-f), (I-x), (I-y), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier. In another aspect, provided is a pharmaceutical composition comprising a compound of Formula (I′), or any related formula such as Formula (I), (II), (III), (IV), (I-a), (I-b), (I-c), u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier. In some embodiments, provided is a pharmaceutical composition comprising a therapeutically effective amount of a compound of Formula (I′), or any related formula such as Formula (I), (II), (III), (IV), (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier.

In another aspect, provided is a method of making a compound of Formula (I), (II), (III), (III), or (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing. In another aspect, provided is a method of making a compound of Formula (I′), or any related formula such as Formula (I), (II), (III), (III), (IV), (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing.

In another aspect, provided is a method of treating a disease or disorder comprising modulating (e.g., reducing) the activity or level of at least one of PIP4K2A, PIP4K2B, and PIP4K2C, comprising administering (a) a compound of Formula (I), (II), (III), or (IV), or any related formula such as Formula (I-a), (I-b), (I-c), (I-d), (I-e), (I-f), (I-g), (I-h), (I-j), (I-k), (I-l), (I-m), (I-o), (I-p), (I-q), (I-r), (I-s), (I-t), (I-u), (I-v), (I-w), (I-x), (I-y), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I), (II), (III), or (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier. In some embodiments, the method comprises reducing the activity of PIP4K2C. In some embodiments, the method comprises selectively reducing the activity of PIP4K2C. In some embodiments, the method comprises selectively reducing the level of PIP4K2C. In some embodiments, the method comprises selectively degrading PIP4K2C. In another aspect, provided is a method of treating a disease or disorder comprising modulating (e.g., reducing) the activity or level of at least one of PIP4K2A, PIP4K2B, and PIP4K2C, comprising administering (a) a compound of Formula (I′), or any related formula such as Formula (I), (II), (III), (IV), (I-a), (I-b), (I-c), (I-d), (I-e), (I-f), (I-g), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I′), or any related formula such as Formula (I), (II), (III), (IV), (I-a), (I-b), (I-c), (I-d), (I-e), (I-f), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier. In some embodiments, the method comprises reducing the activity of PIP4K2C. In some embodiments, the method comprises selectively reducing the activity of PIP4K2C. In some embodiments, the method comprises selectively reducing the level of PIP4K2C. In some embodiments, the method comprises selectively degrading PIP4K2C.

In another aspect, provided is a method of treating a disease or disorder comprising modulating (e.g., reducing) the activity or level of at least one of PIP4K2A, PIP4K2B, and PIP4K2C, comprising administering (a) a therapeutically effective amount of the compound of Formula (I), (II), (III), or (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a therapeutically effective amount of the compound of Formula (I), (II), (III), or (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier. In some embodiments, the method comprises reducing the activity of PIP4K2C. In some embodiments, the method comprises selectively reducing the activity of PIP4K2C. In some embodiments, the method comprises selectively reducing the level of PIP4K2C. In some embodiments, the method comprises selectively degrading PIP4K2C. In some embodiments, the methods are selective for PIP4K2C over PIP4K2A. In some embodiments, the methods are selective for PIP4K2C over PIP4K2B. In some embodiments, the methods are selective for PIP4K2C over both PIP4K2A and PIP4K2B. In another aspect, provided is a method of treating a disease or disorder comprising modulating (e.g., reducing) the activity or level of at least one of PIP4K2A, PIP4K2B, and PIP4K2C, comprising administering (a) a therapeutically effective amount of the compound of Formula (I′), or any related formula such as Formula (I), (II), (III), (IV), (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a therapeutically effective amount of the compound of Formula (I′), or any related formula such as Formula (I), (II), (III), (IV), (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier. In some embodiments, the method comprises reducing the activity of PIP4K2C. In some embodiments, the method comprises selectively reducing the activity of PIP4K2C. In some embodiments, the method comprises selectively reducing the level of PIP4K2C. In some embodiments, the method comprises selectively degrading PIP4K2C. In some embodiments, the methods are selective for PIP4K2C over PIP4K2A. In some embodiments, the methods are selective for PIP4K2C over PIP4K2B. In some embodiments, the methods are selective for PIP4K2C over both PIP4K2A and PIP4K2B.

In another aspect, provided is a method of treating a disease or disorder comprising modulating (e.g., reducing) the level of at least one of PIP4K2A, PIP4K2B, and PIP4K2C, comprising administering (a) a compound of Formula (IV), or any related formula such as (IV-a), (IV-a), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (IV), or any related formula such as (IV-a), (IV-a), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to a subject in need thereof. In some embodiments, the method comprises reducing the level of PIP4K2C. In some embodiments, the method comprises selectively reducing the level of PIP4K2C. In some embodiments, the method comprises degrading PIP4K2C. In some embodiments, the method comprises selectively degrading PIP4K2C.

In another aspect, provided is a method of treating a disease or disorder comprising modulating (e.g., reducing) the level of at least one of PIP4K2A, PIP4K2B, and PIP4K2C, comprising administering (a) a therapeutically effective amount of the compound of Formula (IV), or any related formula such as (IV-a), (IV-a), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a therapeutically effective amount of a compound of Formula (IV), or any related formula such as (IV-a), (IV-a), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to a subject in need thereof. In some embodiments, the method comprises reducing the level of PIP4K2C. In some embodiments, the method comprises degrading PIP4K2C. In some embodiments, the method comprises selectively reducing the level of PIP4K2C. In some embodiments, the method comprises selectively degrading PIP4K2C.

In another aspect, provided is a method of enhancing immune function in a subject in need thereof, comprising administering to a subject (a) a compound of Formula (I), (II), (III), or (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or pharmaceutically acceptable salt of any of the foregoing, or (b) a pharmaceutical composition comprising a compound of Formula (I), (II), (III), or (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier. In another aspect, provided is a method of enhancing immune function in a subject in need thereof, comprising administering to a subject (a) a compound of Formula (I′), or any related formula such as Formula (I), (II), (III), (IV), (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or pharmaceutically acceptable salt of any of the foregoing, or (b) a pharmaceutical composition comprising a compound of Formula (I′), or any related formula such as Formula (I), (II), (III), (IV), (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier.

In some embodiments, provided is a method of enhancing immune function in a subject in need thereof, comprising administering to a subject (a) a therapeutically effective amount of the compound of Formula (I), (II), (III), or (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or pharmaceutically acceptable salt of any of the foregoing, or a (b) pharmaceutical composition comprising a therapeutically effective amount of the compound of Formula (I), (II), (III), or (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier. In some embodiments, provided is a method of enhancing immune function in a subject in need thereof, comprising administering to a subject (a) a therapeutically effective amount of the compound of Formula (I′), or any related formula such as Formula (I), (II), (III), (IV), (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or pharmaceutically acceptable salt of any of the foregoing, or a (b) pharmaceutical composition comprising a therapeutically effective amount of the compound of Formula (I′), or any related formula such as Formula (I), (II), (III), (IV), (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier.

In some aspects, the present invention is directed to methods of stimulating the immune system comprising reducing scaffolding or interaction of at least one of PIP4K2A, PIP4K2B, and PIP4K2C with at least one of PIP4K2A, PIP4K2B, or phosphatidylinositol-4-phosphate 5-kinase (PIP5K) in a subject in need thereof, comprising administering to a subject (a) a compound of Formula (I), (II), (III), or (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or pharmaceutically acceptable salt of any of the foregoing, or (b) a pharmaceutical composition comprising a compound of Formula (I), (II), (III), (III), or (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier. In some embodiments, the method comprises reducing the scaffolding or interaction of PIP4K2C with at least one of PIP4K2A, PIP4K2B, or phosphatidylinositol-4-phosphate 5-kinase (PIP5K). In some aspects, the present invention is directed to methods of stimulating the immune system comprising reducing scaffolding or interaction of at least one of PIP4K2A, PIP4K2B, and PIP4K2C with at least one of PIP4K2A, PIP4K2B, or phosphatidylinositol-4-phosphate 5-kinase (PIP5K) in a subject in need thereof, comprising administering to a subject (a) a compound of Formula (I′), or any related formula such as Formula (I), (II), (III), (IV), (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or pharmaceutically acceptable salt of any of the foregoing, or (b) a pharmaceutical composition comprising a compound of Formula (I′), or any related formula such as Formula (I), (II), (III), (III), (IV), (I-a), (I-b), (I-c), (I-d), (I-e), (I-f), (I-g), (I-h), (I-j), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier. In some embodiments, the method comprises reducing the scaffolding or interaction of PIP4K2C with at least one of PIP4K2A, PIP4K2B, or phosphatidylinositol-4-phosphate 5-kinase (PIP5K).

Any embodiments provided herein of a compound of formula (I) or (I′), or a stereoisomer or tautomer thereof, or a pharmaceutically acceptable salt of any of the foregoing, or any variation or embodiment thereof, apply where applicable to any other formula detailed herein, the same as if each and every embodiment were specifically and individually listed. Thus, it is understood and described that each embodiment provided herein of a compound of formula (I) or (I′), or a stereoisomer or tautomer thereof, or a pharmaceutically acceptable salt of any of the foregoing, or any variation or embodiment thereof, such as embodiments related to Ring 1, Ring 2, Ring 3, Ring 4, Ring 5, Ring A, Ring B, Ring C, R, R¹, R^{2a}, R^{2b}, R^{3a}, R^{3b}, R^{4a}, R^{4b}, R^{5a}, R^{5b}, R^{C}, R^{D}, R^{L}, R^{Q}, R^{V}, R^{w}, R^{x}, R^{y}, R^{z}, Q, U, V, X, W, Linker, L¹, L², L³, and n apply to any one of formulae (i), (II), (III), (IV), (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), the same as if each and every embodiment were specifically and individually listed. It is also understood and described that all such embodiments may be used in any of the pharmaceutical compositions, methods, kits, uses, etc. comprising such compounds or other aspects detailed herein.

As shown in working examples, compounds embraced by Formula (I′) and Formula (I) inhibit the activity or promote the degradation of at least one of PIP4K2A, PIP4K2B, and PIP4K2C. In some embodiments, compounds embraced by Formula (I′) and Formula (I) inhibit the activity or promote the degradation of PIP4K2C.

Without intending to be bound by any particular theory, the compounds of Formula (IV) and any related formula as applicable such as, e.g., (IV-a), (IV-a), (IV-c), (IV-d), (IV-e), (IV-f), (IV-g), (IV-h), (IV-j), (IV-k), or (IV-l), are believed to cause degradation of at least one of PIP4K2A, PIP4K2B, and PIP4K2C by recruitment of cells' Ubiquitin/Proteasome System, whose function is to routinely identify and remove damaged proteins, into close proximity with PIP4K2A, PIP4K2B, or PIP4K2C as a result of binding between PIP4K2A, PIP4K2B, or PIP4K2C, and the targeting ligand. After destruction of a PIP4K2A, PIP4K2B, or PIP4K2C protein, the degrader is released and continues to be active. Thus, by engaging and exploiting the body's own natural protein disposal system, the compounds of the present invention may represent a potential improvement over current small molecule inhibitors of PIP4K2A, PIP4K2B, and PIP4K2C and may overcome one or more limitations regarding their use. Therefore, effective intracellular concentrations of the degraders may be significantly lower than for small molecule PIP4K2A, PIP4K2B, or PIP4K2C inhibitors. Also, although PIP4K2A, PIP4K2B, and/or PIP4K2C genetic knockdown or knockout can be used to reduce the cellular concentration or amount of these proteins, it may be preferable to post-translationally disrupt, degrade, or destabilize PIP4K2A, PIP4K2B, and/or PIP4K2C proteins. Targeting proteins directly, rather than via the DNA or mRNA molecules that encode them, is a more direct and rapid method for reducing the scaffolding function of PIP4K proteins. Hence, degradation can allow some PIP4K2A, PIP4K2B, and/or PIP4K2C catalytic function to proceed while reducing the non-catalytic functions such as scaffolding between PIP4K2A, PIP4K2B, and/or PIP4K2C proteins and other cellular proteins and structures. Collectively, the present compounds may represent a set of new chemical tools for at least one of PIP4K2A, PIP4K2B, and PIP4K2C knockdown and may provide a potential treatment modality for PIP4K2A, PIP4K2B, and/or PIP4K2C-associated cancers and autophagy-dependent diseases (e.g., neurodegenerative disorders, insulin). In some instances, the present compounds may represent a set of new chemical tools for PIP4K2C knockdown and may provide a potential treatment modality for PIP4K2C-associated cancers and autophagy-dependent diseases (e.g., neurodegenerative disorders, insulin). The compounds may be used to enhance immune functions as described in, e.g., US20220168402A1 and Wang et al., Cell Rep. 27:1991-2001 (2019), each of which is incorporated herein by reference in its entirety. Inhibitors and degraders of PIP4K protein and methods for treating diseases or conditions by modulating the level or activity of at least one of PIP4K2A, PIP4K2B, and PIP4K2C are also disclosed in WO 2022/246025, herein incorporated by reference in its entirety.

### BRIEF DESCRIPTION OF THE FIGURES

FIG. 1 shows modulation of phagocytosis by Compound U. The degradation of PIP4K2C by Compound U in human dendritic cells enhanced their phagocytosis of E. coli bioparticles, emphasizing the intrinsic immunological benefits of PIP4K2C targeting in myeloid cells.

FIG. 2A shows the ability of Compound U to reduce tumor growth in the mouse syngeneic colorectal cancer model MC38-OVA. A marked reduction in tumor growth was observed in the treated group when compared to the vehicle control group. FIG. 2B shows the survival of the mice treated with Compound U and mice treated with the vehicle control.

FIG. 3 shows that degradation of PIP4K2C by Compound U enhances phagocytosis of E. coli bioparticles in human dendritic cells. The enhanced phagocytosis effect is not seen when using a PIP4K2C inhibitor compound that does not cause degradation, demonstrating the unique impact of a degrader of PIP4K2C over an inhibitory compound alone.

FIG. 4 shows that degradation of PIP4K2C by Compound U in dendritic cells leads to an increased uptake of dying tumor-derived material, adding to the enhanced engulfment of PIP4K2C-deficient tumor cells by immune cells.

FIG. 5A shows that Compound U degrades endogenous PIP4K2C in vivo in rats.

FIG. 5B shows that Compound U degrades endogenous PIP4K2C in vivo in dogs.

### DETAILED DESCRIPTION

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, “about X” includes and describes “X” per se.

“Alkyl” as used herein refers to and includes, unless otherwise stated, a saturated linear (i.e., unbranched) or branched univalent hydrocarbon chain or combination thereof, having the number of carbon atoms designated (i.e., C₁₋₁₀ means one to ten carbon atoms). Particular alkyl groups are those having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Particular C₁-C₄ alkyl groups include C₁-C₃ alkyl groups. Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Alkylene” as used herein refers to the same residues as alkyl, but having bivalency. Particular alkylene groups are those having 1 to 10 carbon atoms (a “C₁-C₁₀ alkylene”), having 6 to 10 carbon atoms (a “C₆-C₁₀ alkylene”), having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

“Carbocyclyl” as used herein refers to and includes, unless otherwise stated, saturated and partially unsaturated hydrocarbon structures, having the number of carbon atoms designated (i.e., C₃-C₁₀ means three to ten carbon atoms). Cycloalkyl can consist of one ring (e.g., cyclohexyl), or multiple rings (e.g., adamantyl). In polycyclic ring systems, one or more of the fused rings can be carbocyclyl or aryl (e.g., spiro[4.5]decanyl, or 1,2,3,4-tetrahydronaphthalenyl), but excludes heterocyclyl or heteroaryl groups. A carbocyclyl comprising more than one ring may be fused, spiro or bridged, or combinations thereof. Particular carbocyclyl groups are those having from 3 to 12 annular carbon atoms (a “C₃₋₁₂ carbocyclyl”), having 3 to 10 annular carbon atoms (a “C₃₋₁₀ carbocyclyl”), having 3 to 8 annular carbon atoms (a “C₃-C₈ carbocyclyl”), having 3 to 6 annular carbon atoms (a “C₃-C₆ carbocyclyl”), or having from 3 to 4 annular carbon atoms (a “C₃-C₄ carbocyclyl”). A carbocyclyl group having more than one ring where at least one ring is aromatic may be connected to the parent structure at either an aromatic ring position or at a non-aromatic ring position. In one variation, a carbocyclyl group having more than one ring where at least one ring is aromatic is connected to the parent structure at an aromatic ring position. In another variation, a carbocyclyl group having more than one ring where at least one ring is aromatic is connected to the parent structure at a non-aromatic ring position.

“Cycloalkyl” as used herein refers to and includes, unless otherwise stated, saturated cyclic hydrocarbon structures, having the number of carbon atoms designated (i.e., C₃-C₁₀ means three to ten carbon atoms). Cycloalkyl can consist of one ring (e.g., cyclohexyl), or multiple rings (e.g., adamantyl). In polycyclic ring systems, one or more of the fused rings can be cycloalkyl or aryl (e.g., spiro[4.5]decanyl, or 1,2,3,4-tetrahydronaphthalenyl), but excludes heterocyclyl or heteroaryl groups. A cycloalkyl comprising more than one ring may be fused, spiro or bridged, or combinations thereof. Particular cycloalkyl groups are those having from 3 to 12 annular carbon atoms (a “C₃₋₁₂ cycloalkyl”), having 3 to 10 annular carbon atoms (a “C₃₋₁₀ cycloalkyl”), having 3 to 8 annular carbon atoms (a “C₃-C₅ cycloalkyl”), having 3 to 6 annular carbon atoms (a “C₃-C₆ cycloalkyl”), or having from 3 to 4 annular carbon atoms (a “C₃-C₄ cycloalkyl”). Examples of cycloalkyl include, but are not limited to, cyclopropyl, cyclobutyl, cyclopentyl, cyclohexyl, cycloheptyl, norbornyl, and the like. A cycloalkyl group having more than one ring where at least one ring is aromatic may be connected to the parent structure at either an aromatic ring position or at a non-aromatic ring position. In one variation, a cycloalkyl group having more than one ring where at least one ring is aromatic is connected to the parent structure at an aromatic ring position. In another variation, a cycloalkyl group having more than one ring where at least one ring is aromatic is connected to the parent structure at a non-aromatic ring position.

“Aryl” or “Ar” as used herein refers to an unsaturated aromatic carbocyclic group having a single ring (e.g., phenyl) or multiple condensed rings (e.g., naphthyl or anthryl) which condensed rings must be aromatic. Particular aryl groups are those having from 6 to 14 annular carbon atoms (a “C₆-C₁₄ aryl”).

“Heteroaryl” as used herein refers to an unsaturated aromatic cyclic group having from 1 to 14 annular carbon atoms and at least one annular heteroatom, including but not limited to heteroatoms such as nitrogen, oxygen and sulfur. A heteroaryl group may have a single ring (e.g., pyridyl, furyl) or multiple condensed rings (e.g., indolizinyl, benzothienyl) which condensed rings must be aromatic. Particular heteroaryl groups are 5 to 14-membered rings having 1 to 12 annular carbon atoms and 1 to 6 annular heteroatoms independently selected from nitrogen, oxygen and sulfur, 5 to 10-membered rings having 1 to 8 annular carbon atoms and 1 to 4 annular heteroatoms independently selected from nitrogen, oxygen and sulfur, or 5, 6 or 7-membered rings having 1 to 5 annular carbon atoms and 1 to 4 annular heteroatoms independently selected from nitrogen, oxygen and sulfur. In one variation, particular heteroaryl groups are monocyclic aromatic 5-, 6- or 7-membered rings having from 1 to 6 annular carbon atoms and 1 to 4 annular heteroatoms independently selected from nitrogen, oxygen and sulfur. In another variation, particular heteroaryl groups are polycyclic aromatic rings having from 1 to 12 annular carbon atoms and 1 to 6 annular heteroatoms independently selected from nitrogen, oxygen and sulfur. A heteroaryl group may be connected to the parent structure at a ring carbon atom or a ring heteroatom.

“Heterocycle”, “heterocyclic”, or “heterocyclyl” as used herein refers to a saturated or partially unsaturated non-aromatic cyclic group having a single ring or multiple condensed rings, and having from 1 to 14 annular carbon atoms and from 1 to 6 annular heteroatoms, such as nitrogen, sulfur or oxygen, and the like. A heterocycle comprising more than one ring may be fused, bridged or spiro, or any combination thereof. In fused ring systems, one or more of the fused rings can be cycloalkyl, aryl, or heteroaryl groups. The heterocyclyl group may be optionally substituted independently with one or more substituents described herein. Particular heterocyclyl groups are 3 to 14-membered rings having 1 to 13 annular carbon atoms and 1 to 6 annular heteroatoms independently selected from nitrogen, oxygen and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur. A heterocyclyl group having more than one ring where at least one ring is aromatic may be connected to the parent structure at either an aromatic ring position or at a non-aromatic ring position. In one variation, a heterocyclyl group having more than one ring where at least one ring is aromatic is connected to the parent structure at an aromatic ring position. In another variation, a heterocyclyl group having more than one ring where at least one ring is aromatic is connected to the parent structure at a non-aromatic ring position.

“Halo” or “halogen” refers to elements of the Group 7 of the periodic table. Preferred halo groups include fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbonyl” refers to the group C═O.

“Oxo” refers to the moiety ═O.

“Optionally substituted” unless otherwise specified means that a group may be unsubstituted or substituted with one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

It is understood that aspects and embodiments described herein as “comprising” include “consisting of” and “consisting essentially of” embodiments.

The term “pharmaceutically acceptable salt”, as used herein, of a given compound refers to salts that retain the biological effectiveness and properties of the given compound and which are not biologically or otherwise undesirable. “Pharmaceutically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. See, e.g., Handbook of Pharmaceutical Salts Properties, Selection, and Use, International Union of Pure and Applied Chemistry, John Wiley & Sons (2008), which is incorporated herein by reference. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, trifluoroacetic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Some of the compounds provided herein may exist as tautomers. Tautomers are in equilibrium with one another. By way of illustration, amide containing compounds may exist in equilibrium with imidic acid tautomers. Regardless of which tautomer is shown and regardless of the nature of the equilibrium among tautomers, the compounds of this disclosure are understood by one of ordinary skill in the art to comprise both amide and imidic acid tautomers. Thus, for example, amide-containing compounds are understood to include their imidic acid tautomers. Likewise, imidic-acid containing compounds are understood to include their amide tautomers.

The compounds of the present disclosure, or their pharmaceutically acceptable salts, may include an asymmetric center and may thus give rise to enantiomers, diastereomers, and other stereoisomeric forms that may be defined, in terms of absolute stereochemistry, as (R)- or (S)- (or as (D)- or (L)- for amino acids). The present disclosure is meant to include all such possible isomers, as well as their racemic and optically pure forms and mixtures thereof in any ratio. Optically active (+) and (−), (R)- and (S)-, or (D)- and (L)-isomers may be prepared using chiral synthons or chiral reagents, or may be resolved using conventional techniques, for example, chromatography and/or fractional crystallization. Conventional techniques for the preparation/isolation of individual enantiomers include chiral synthesis from a suitable optically pure precursor or the resolution of the racemate (or the racemate of a salt or derivative) using, for example, chiral high pressure liquid chromatography (HPLC), and chiral supercritical fluid chromatography (SFC). When the compounds described herein contain olefinic double bonds or other centers of geometric asymmetry, unless specified otherwise, it is intended that the present disclosure includes both E and Z geometric isomers. Likewise, cis- and trans- are used in their conventional sense to describe relative spatial relationships.

A “stereoisomer” refers to a compound made up of the same atoms bonded by the same bonds, but having different three-dimensional structures, which are not interchangeable. The present disclosure contemplates various stereoisomers, or mixtures thereof (e.g., racemic mixtures), and includes “enantiomers,” which refers to two stereoisomers whose structures are non-superimposable mirror images of one another. “Diastereomers” are stereoisomers that have at least two asymmetric atoms, but which are not mirror images of each other. Where enantiomeric and/or diastereomeric forms exist of a given structure, flat bonds indicate that all stereoisomeric forms of the depicted structure may be present, e.g.,

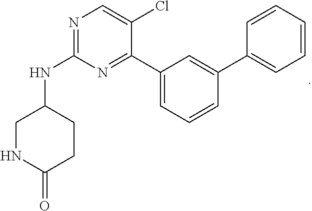 [SMILES: O=C1CCC(Nc2ncc(Cl)c(-c3cccc(-c4ccccc4)c3)n2)CN1]

Where enantiomeric and/or diastereomeric forms exist of a given structure, unless indicated otherwise, wedged or hashed bonds without “R”, “S”, or “abs” indicate the composition is made up of at least 90% by weight of a mixture of isomers with known relative stereochemistry, e.g.,

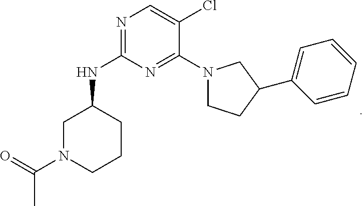 [SMILES: CC(=O)N1CCC[C@H](Nc2ncc(Cl)c(N3CCC(c4ccccc4)C3)n2)C1]

Where enantiomeric and/or diastereomeric forms exist of a given structure, wedged or hashed bonds with “R”, “S”, or “abs” indicate the composition is made up of at least 90% by weight of a single enantiomer or diastereomer with known absolute stereochemistry, e.g.,

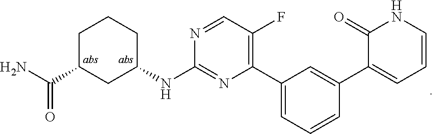 [SMILES: NC(=O)[C@@H]1CCC[C@H](Nc2ncc(F)c(-c3cccc(-c4ccc[nH]c4=O)c3)n2)C1]

In some instances, diastereomeric mixtures are designated using “r” and/or “s” configurations. In such instances, relative stereochemistry is designated by lowercase (“r” or “s”), and absolute stereochemistry is designated by uppercase (“R” or “S”).

The term “disease” as used herein is regarded as a state of health of a subject, wherein the subject cannot maintain homeostasis, and/or wherein, if the disease is not ameliorated, then the subject's health further deteriorates. In contrast, a “disorder” in a subject is a state of health in which the subject is able to maintain homeostasis, but the subject's health is less favorable than it in the absence of the disorder. A disorder does not necessarily cause a further decrease in the subject's health, when left untreated.

The term “subject” (or “patient”) as used herein includes mammals prone to or suffering from the indicated disease or disorder e.g., a human or a non-human mammal. In some embodiments, the subject is a human. In some embodiments, the subject is a companion animal such as dogs and cats; in some embodiments, the subject is a livestock animal such as cows, horses, sheep, goats, and pigs; in some embodiments, the subject may be other domesticated and wild animals. A subject “in need of” treatment according to the present invention may be “suffering from or suspected of suffering from” a specific disease or disorder; may have been positively diagnosed with the disease or disorder; or otherwise presents with a sufficient number of risk factors or combination of symptoms such that a medical professional could diagnose the subject with the disease or disorder or suspect that the subject was suffering from the disease or disorder.

As used herein, the term, “therapeutically effective amount” is an amount of a compound, or a tautomer or stereoisomer thereof, or a pharmaceutically acceptable salt of any of the foregoing; or a composition including a compound, or a tautomer or stereoisomer thereof, or a pharmaceutically acceptable salt of any of the foregoing, which is effective in yielding the desired therapeutic response in a particular subject in need thereof. Therefore, the term “therapeutically effective amount” includes the amount of a compound, or a tautomer or stereoisomer thereof, or a pharmaceutically acceptable salt of any of the foregoing, that when administered, induces an improvement in the disease or disorder to be treated, or is sufficient to prevent or slow development or progression of the disease or disorder, or alleviate to some extent one or more of the symptoms of the disease or disorder being treated in a subject, or kills or inhibits the growth of diseased cells (e.g., cancer cells, or autophagy-dependent disease cells), or reduces the amount of at least one of PIP4K2A, PIP4K2B, and PIP4K2C (e.g., reduces the amount of at least PIP4K2C) in diseased cells.

As used herein, the term “neurodegenerative diseases and disorders” refers to conditions characterized by progressive degeneration and/or death of nerve cells, including problems with movement or mental functioning. Representative examples include Alzheimer's disease (AD) and related dementias, Parkinson's disease (PD) and related dementias, prion disease, motor neuron diseases (MND), Huntington's disease (HD), Pick's syndrome, spinocerebellar ataxia (SCA), spinal muscular atrophy (SMA), primary progressive aphasia (PPA), amyotrophic lateral sclerosis (ALS), traumatic brain injury (TBI), multiple sclerosis (MS), and other dementias (e.g., vascular dementia (VaD), Lewy body dementia (LBD), semantic dementia, and frontotemporal lobar dementia (FTD)).

As used herein, the term “autoimmune disease” refers to conditions where the immune system produces antibodies that attack normal body tissues. Representative examples of autoimmune diseases include, without limitation, autoimmune hematological disorders (e.g., hemolytic anemia, aplastic anemia, anhidrotic ectodermal dysplasia, pure red cell anemia and idiopathic thrombocytopenia), Sjogren's syndrome, Hashimoto thyroiditis, rheumatoid arthritis, juvenile (type 1) diabetes, polymyositis, scleroderma, Addison's disease, lupus including systemic lupus erythematosus, vitiligo, pernicious anemia, glomerulonephritis, pulmonary fibrosis, celiac disease, polymyalgia rheumatica, multiple sclerosis, ankylosing spondylitis, alopecia areata, vasculitis, autoimmune uveoretinitis, lichen planus, bullous pemphigus, pemphigus vulgaris, pemphigus foliaceus, paraneoplastic pemphigus, myasthenia gravis, immunoglobulin A nephropathy, Wegener granulomatosis, autoimmune oophoritis, sarcoidosis, rheumatic carditis, ankylosing spondylitis, Grave's disease, autoimmune thrombocytopenic purpura, psoriasis, psoriatic arthritis, dermatitis herpetiformis, ulcerative colitis, and temporal arteritis, and the like.

As used herein, the term “cell proliferative disease or disorder” refers to a condition characterized by dysregulated or abnormal cell growth, or both, including noncancerous conditions such as neoplasms, precancerous conditions, benign tumors, and cancer.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), adenocarcinoma, small cell carcinoma, large cell carcinoma, squamous cell carcinoma, and mesothelioma. Lung cancer can include “scar carcinoma”, bronchoalveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, a compound treats non-metastatic or metastatic lung cancer (e.g., NSCLC, ALK-positive NSCLC, NSCLC harboring ROS1 rearrangement, lung adenocarcinoma, and squamous cell lung carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

In some embodiments, the cancer is a hematological cancer e.g., leukemia, lymphoma or multiple myeloma.

### Compounds of Formula (I′) or (I)

In one aspect, provided is a compound of Formula (I′)

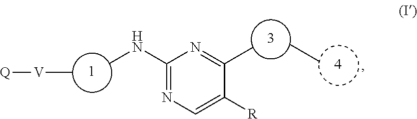

or a tautomer or stereoisomer thereof, or a pharmaceutically acceptable salt of any of the foregoing, wherein:
R is halo;
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Ring 3 is a monocyclic ring selected from the group consisting of 3- to 6-membered cycloalkyl optionally substituted with one or more R^{3a}; 4- to 6-membered heterocyclyl optionally substituted with one or more R^{3a}; phenyl optionally substituted with one or more R^{3b}; or 5- to 6-membered heteroaryl optionally substituted with one or more R^{3b};
Ring 1 is a 4- to 8-membered cycloalkyl or saturated 4- to 8-membered heterocyclyl, each of which is optionally substituted with one or more R¹;
V is a bond, —C₁₋₄alkylene-, —C(O)—, —C(O)O-#, —OC(O)#—C₁₋₄alkylene-C(O)-#, —C(O)N(R^{V})-#, —N(R^{V})C(O)-#, —OC(O)N(R^{V})-#, —N(R^{V})—C(O)O#, —C₁₋₄alkylene-C(O)N(R)-# or —C₁₋₄alkylene-N(R^{V})C(O)-#, wherein # indicates the point of attachment to Ring 1; and
Q is R^{Q}, or Ring 5,
wherein Ring 5 is selected from the group consisting of 3- to 10-membered cycloalkyl optionally substituted with one or more R^{5a}, 4- to 12-membered heterocyclyl optionally substituted with one or more R^{5a}, and 5- to 12-membered heteroaryl optionally substituted with one or more R^{b};
or
Q is of Formula (i)

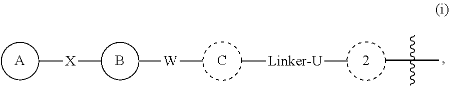

wherein:
Ring A is selected from the group consisting of

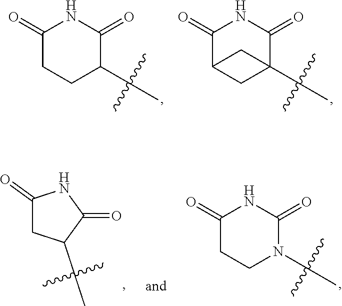 [SMILES: *C1CCC(=O)NC1=O ; *C12CC(C1)C(=O)NC2=O ; *C1CC(=O)NC1=O ; *N1CCC(=O)NC1=O]

each of which is optionally substituted with one or more C₁₋₄alkyl or halo;
X is a bond, —O—, —NH—, or —NHC(O)—;
Ring B is selected from the group consisting of

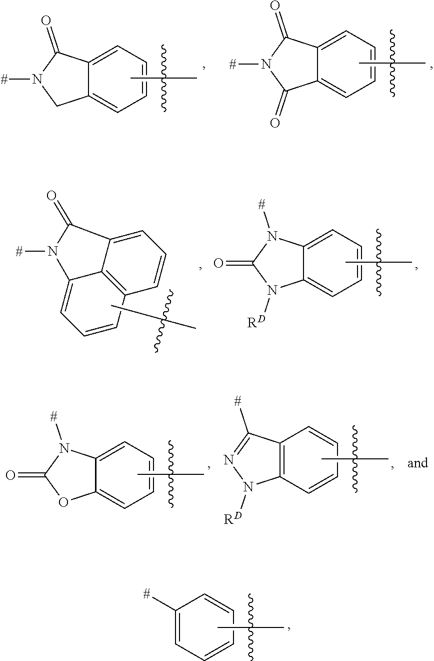

which is optionally substituted with one or more halo, C₁₋₄alkyl, C₁₋₄haloalkyl, or OH, and wherein R^{D} is H or C₁₋₄alkyl, wherein # indicates attachment to X;
W is a bond, —O—, —NH—, or —NHC(O)—;
Ring C is optional, and when present is 4- to 12-membered heterocyclyl optionally substituted with one or more R^{C};
   Linker is a bond, —O—, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein
      * indicates the point of attachment to Ring C when Ring C is present, and * indicates the point of attachment to W when Ring C is absent; and
      ** indicates the point of attachment to U when Ring 2 is present, and ** indicates the point of attachment to V when Ring 2 is absent;
      L¹ is a bond or C₁₋₆ alkylene;
      L² is a 3- to 10-membered cycloalkyl or 4- to 12-membered heterocyclyl optionally substituted with one or more R^{L}; and
      L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y});
   U is absent when Ring 2 is absent, and is a bond or C(O) when Ring 2 is present;
   Ring 2 is optional, and when present is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b};
each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{2a}, R^{3a}, R^{4a}, and R^{5a} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{2b}, R^{3b}, R^{4b}, and R^{5b} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C₁₋₄hydroxyalkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R^{C} is independently halo, OH, C₁₋₄alkyl, or C₁₋₄haloalkyl;
each R^{L} is independently halo, OH, C₁₋₄alkyl, or C₁₋₄haloalkyl;
each R^{Q} is independently H, halo, OH, C₁₋₄alkyl, or C₁₋₄haloalkyl;
each R^{V} is independently H, halo, OH, C₁₋₄alkyl, or C₁₋₄haloalkyl;
each R^{w} is independently 3- to 10-membered cycloalkyl, phenyl optionally substituted with halo, or 4- to 12-membered heterocyclyl optionally substituted with hydroxyl, C₁₋₄ hydroxyalkyl, or C₁₋₄aminoalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ-C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, 3- to 10-membered cycloalkyl, or C₁₋₄alkylene-S(O)ₙ-C₁₋₄alkyl; and
each n is independently 0, 1, or 2;
   wherein:
(a) Ring 1 comprises an intraannular amide, or
(b) V comprises an amide or carbamate, or
(c) V is taken together with an atom of Ring 1, Ring 2, or Ring 5 to which it is attached, to form an amide or carbamate;
   and wherein:
when Ring 3 is 5-membered heteroaryl, Ring 4 is present and is not cycloalkyl or tetrahydropyranyl;
   and wherein:
when Ring 1 is 8-membered heterocyclyl, Ring 3 is phenyl substituted with CN, Ring 4 is absent, Q is R^{Q}, and V is —OC(O)#, then R^{Q} is not tert-butyl.

In one aspect, provided is a compound of Formula (I′)

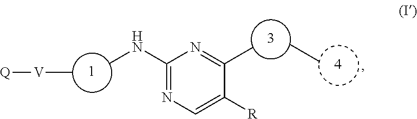

or a tautomer or stereoisomer thereof, or a pharmaceutically acceptable salt of any of the foregoing, wherein:
R is halo;
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Ring 3 is a monocyclic ring selected from the group consisting of 3- to 6-membered cycloalkyl optionally substituted with one or more R^{3a}; 4- to 6-membered heterocyclyl optionally substituted with one or more R^{3a}; phenyl optionally substituted with one or more R^{3b}; or 5- to 6-membered heteroaryl optionally substituted with one or more R^{3b};
Ring 1 is a 4- to 8-membered cycloalkyl or saturated 4- to 8-membered heterocyclyl, each of which is optionally substituted with one or more R¹;
V is a bond, —C₁₋₄alkylene-, —C(O)—, —C(O)O-#, —OC(O)#—C₁₋₄alkylene-C(O)-#, —C(O)N(R^{V})-#, —N(R^{V})C(O)-#, —OC(O)N(R^{V})-#, —N(R^{V})—C(O)O #, —C₁₋₄alkylene-C(O)N(R^{V})-# or —C₁₋₄alkylene-N(R^{V})C(O)-#, wherein # indicates the point of attachment to Ring 1; and
Q is R^{Q}, or Ring 5,
wherein Ring 5 is selected from the group consisting of 3- to 10-membered cycloalkyl optionally substituted with one or more R^{5a}, 4- to 12-membered heterocyclyl optionally substituted with one or more R^{5a}, and 5- to 12-membered heteroaryl optionally substituted with one or more R^{5b};
or
Q is of Formula (i)

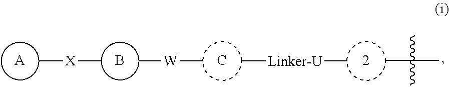

wherein:
Ring A is selected from the group consisting of

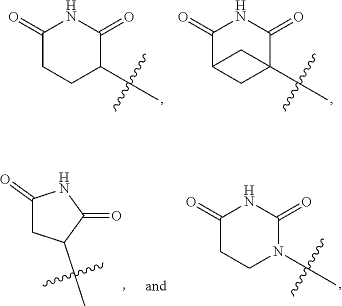 [SMILES: *C1CCC(=O)NC1=O ; *C12CC(C1)C(=O)NC2=O ; *C1CC(=O)NC1=O ; *N1CCC(=O)NC1=O]

each of which is optionally substituted with one or more C₁₋₄alkyl or halo;
X is a bond, —O—, —NH—, or —NHC(O)—;
Ring B is selected from the group consisting of

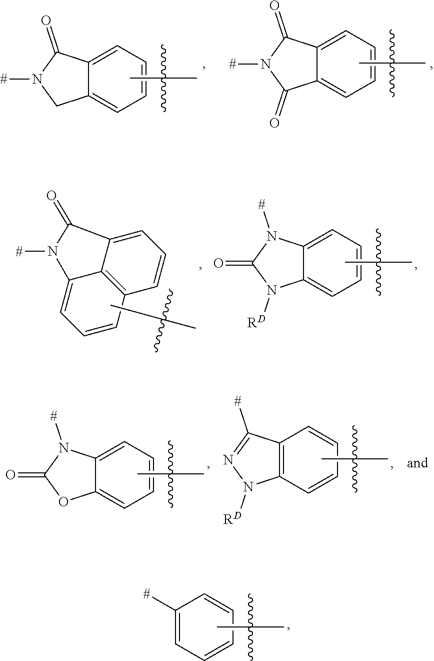

each of which is optionally substituted with one or more halo, C₁₋₄alkyl, C₁₋₄haloalkyl, or OH, and wherein R^{D} is H or C₁₋₄alkyl, wherein # indicates attachment to X;
W is a bond, —O—, —NH—, or —NHC(O)—;
Ring C is optional, and when present is 4- to 12-membered heterocyclyl optionally substituted with one or more R^{C};
   Linker is a bond, —O—, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein
      * indicates the point of attachment to Ring C when Ring C is present, and * indicates the point of attachment to W when Ring C is absent; and
      ** indicates the point of attachment to U when Ring 2 is present, and ** indicates the point of attachment to V when Ring 2 is absent;
      L¹ is a bond or C₁₋₆ alkylene;
      L² is a 3- to 10-membered cycloalkyl or 4- to 12-membered heterocyclyl optionally substituted with one or more R^{L}; and
      L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y});
   U is absent when Ring 2 is absent, and is a bond or C(O) when Ring 2 is present;
   Ring 2 is optional, and when present is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b};
each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)₁—C₁₋₄alkyl;
each R^{2a}, R^{3a}, R^{4a}, and R^{5a} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{2b}, R^{3b}, R^{4b}, and R^{5b} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C₁₋₄hydroxyalkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R^{C} is independently halo, OH, C₁₋₄alkyl, or C₁₋₄haloalkyl;
each R^{L} is independently halo, OH, C₁₋₄alkyl, or C₁₋₄haloalkyl;
each R^{Q} is independently H, halo, OH, C₁₋₄alkyl, or C₁₋₄haloalkyl;
each R^{V} is independently H, halo, OH, C₁₋₄alkyl, or C₁₋₄haloalkyl;
each R^{w} is independently 3- to 10-membered cycloalkyl, phenyl optionally substituted with halo, or 4- to 12-membered heterocyclyl optionally substituted with hydroxyl, C₁. 4hydroxyalkyl, or C₁₋₄aminoalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₐ—C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, 3- to 10-membered cycloalkyl, or C₁₋₄alkylene-S(O)ₙ—C₁₋₄alkyl; and
each n is independently 0, 1, or 2;
   wherein:
(a) Ring 1 comprises an intraannular amide, or
(b) V comprises an amide or carbamate, or
(c) V is taken together with an atom of Ring 1, Ring 2, or Ring 5 to which it is attached, to form an amide or carbamate;
   and wherein:
when Ring 3 is 5-membered heteroaryl, Ring 4 is present and is not cycloalkyl or tetrahydropyranyl.

In one aspect, provided is a compound of Formula (I):

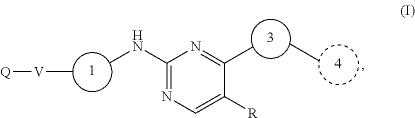

or a tautomer or stereoisomer thereof, or a pharmaceutically acceptable salt of any of the foregoing, wherein:
R is halo;
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Ring 3 is a monocyclic ring selected from the group consisting of 3- to 6-membered cycloalkyl optionally substituted with one or more R^{3a}; 4- to 6-membered heterocyclyl optionally substituted with one or more R^{3a}; phenyl optionally substituted with one or more R^{3b}; or 5- to 6-membered heteroaryl optionally substituted with one or more R^{3b};
Ring 1 is a 4- to 8-membered cycloalkyl or saturated 4- to 8-membered heterocyclyl, each of which is optionally substituted with one or more R¹, wherein each 4- to 8-membered heterocyclyl of Ring 1 comprises at least one heteroatom selected from N and O;
V is a bond, —C₁₋₄alkylene-, —C(O)—, —C₁₋₄alkylene-C(O)-#, —C(O)N(R^{V})-#, —N(R^{V})C(O)-#, —C₁₋₄alkylene-C(O)N(R^{V})-# or —C₁₋₄alkylene-N(R^{V})C(O)-#, wherein # indicates the point of attachment to Ring 1; and
Q is R^{Q}, or Ring 5,
wherein Ring 5 is selected from the group consisting of 3- to 10-membered cycloalkyl optionally substituted with one or more R^{5a}, 4- to 12-membered heterocyclyl optionally substituted with one or more R^{5a}, and 5- to 12-membered heteroaryl optionally substituted with one or more R^{5b},
or
Q is of Formula (i)

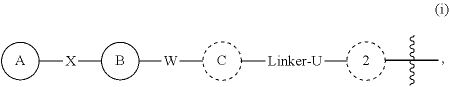

wherein:
Ring A is selected from the group consisting of

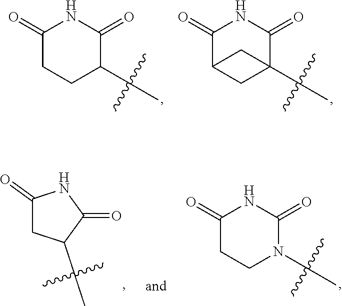 [SMILES: *C1CCC(=O)NC1=O ; *C12CC(C1)C(=O)NC2=O ; *C1CC(=O)NC1=O ; *N1CCC(=O)NC1=O]

each of which is optionally substituted with one or more C₁₋₄alkyl or halo;
X is a bond, —O—, —NH—, or —NHC(O)—;
Ring B is selected from the group consisting of

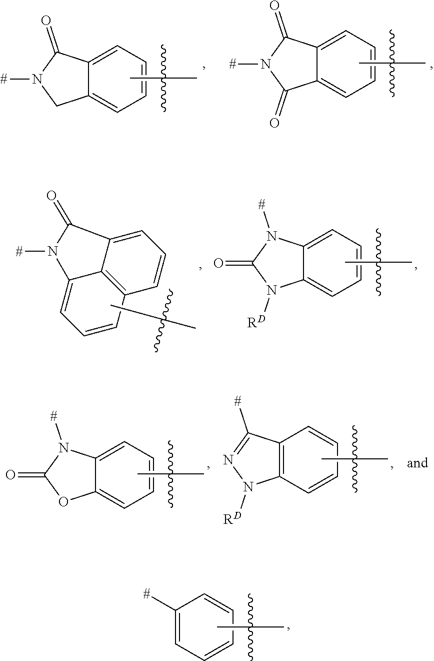

each of which is optionally substituted with one or more halo, C₁₋₄alkyl, C₁₋₄haloalkyl, or OH, and wherein R^{D} is H or C₁₋₄alkyl, wherein # indicates attachment to X;
W is a bond, —O—, —NH—, or —NHC(O)—;
Ring C is optional, and when present is 4- to 12-membered heterocyclyl optionally substituted with one or more R^{C};
   Linker is a bond, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein
      * indicates the point of attachment to Ring C when Ring C is present, and * indicates the point of attachment to W when Ring C is absent; and
      ** indicates the point of attachment to U when Ring 2 is present, and ** indicates the point of attachment to V when Ring 2 is absent;
      L¹ is a bond or C₁₋₆ alkylene;
      L² is a 3- to 10-membered cycloalkyl or 4- to 12-membered heterocyclyl optionally substituted with one or more R^{L}; and
      L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y});
   U is absent when Ring 2 is absent, and is a bond or C(O) when Ring 2 is present;
   Ring 2 is optional, and when present is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b};
each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{2a}, R^{3a}, R^{4a}, and R^{5a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₐN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{2b}, R^{3b}, R^{4b}, and R^{5b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R^{C} is independently halo, OH, C₁₋₄alkyl, or C₁₋₄haloalkyl;
each R^{L} is independently halo, OH, C₁₋₄alkyl, or C₁₋₄haloalkyl;
each R^{Q} is independently H, halo, OH, C₁₋₄alkyl, or C₁₋₄haloalkyl;
each R^{V} is independently halo, OH, C₁₋₄alkyl, or C₁₋₄haloalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ-C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, or C₁₋₄alkylene-S(O)ₙ-C₁₋₄alkyl; and
each n is independently 0, 1, or 2;
   wherein:
(a) Ring 1 comprises an intraannular amide, or
(b) V comprises an amide, or
(c) V is taken together with an atom of Ring 1, Ring 2, or Ring 5 to which it is attached, to form an amide;
   and wherein:
when Ring 3 is 5-membered heteroaryl, Ring 4 is present and is not cycloalkyl or tetrahydropyranyl;
   and wherein:
when Ring 1 is 8-membered heterocyclyl, Ring 3 is phenyl substituted with CN, Ring 4 is absent, Q is R^{Q}, and V is —OC(O)#, then R^{Q} is not tert-butyl.

In one aspect, provided is a compound of Formula (I):

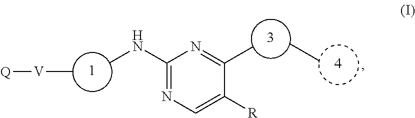

or a tautomer or stereoisomer thereof, or a pharmaceutically acceptable salt of any of the foregoing, wherein:
R is halo;
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Ring 3 is a monocyclic ring selected from the group consisting of 3- to 6-membered cycloalkyl optionally substituted with one or more R^{3a}; 4- to 6-membered heterocyclyl optionally substituted with one or more R^{3a}; phenyl optionally substituted with one or more R^{3b}; or 5- to 6-membered heteroaryl optionally substituted with one or more R^{3b};
Ring 1 is a 4- to 8-membered cycloalkyl or saturated 4- to 8-membered heterocyclyl, each of which is optionally substituted with one or more R¹, wherein each 4- to 8-membered heterocyclyl of Ring 1 comprises at least one heteroatom selected from N and O;
V is a bond, —C₁₋₄alkylene-, —C(O)—, —C₁₋₄alkylene-C(O)-#, —C(O)N(R^{V})-#, —N(R^{V})C(O)-#, —C₁₋₄alkylene-C(O)N(R^{V})-# or —C₁₋₄alkylene-N(R^{V})C(O)-#, wherein # indicates the point of attachment to Ring 1; and
Q is R^{Q}, or Ring 5,
wherein Ring 5 is selected from the group consisting of 3- to 10-membered cycloalkyl optionally substituted with one or more R^{5a}, 4- to 12-membered heterocyclyl optionally substituted with one or more R^{5a}, and 5- to 12-membered heteroaryl optionally substituted with one or more R^{5b};
or
Q is of Formula (i)

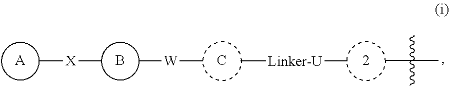

wherein:
Ring A is selected from the group consisting of

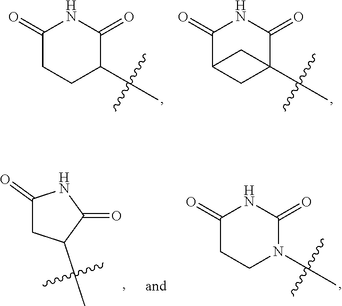 [SMILES: *C1CCC(=O)NC1=O ; *C12CC(C1)C(=O)NC2=O ; *C1CC(=O)NC1=O ; *N1CCC(=O)NC1=O]

each of which is optionally substituted with one or more C₁₋₄alkyl or halo;
X is a bond, —O—, —NH—, or —NHC(O)—;
Ring B is selected from the group consisting of

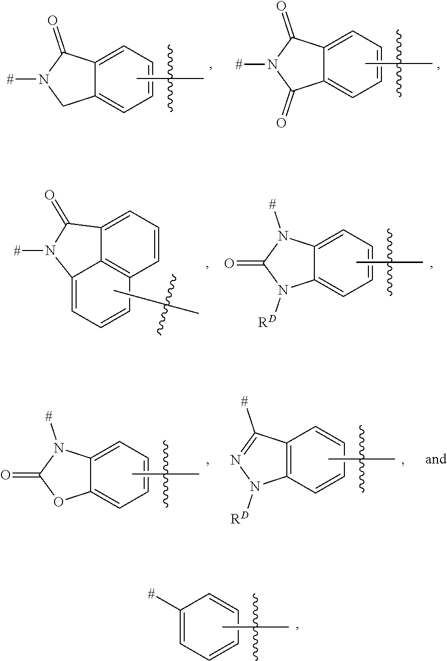

each of which is optionally substituted with one or more halo, C₁₋₄alkyl, C₁₋₄haloalkyl, or OH, and wherein R^{D} is H or C₁₋₄alkyl, wherein # indicates attachment to X;
W is a bond, —O—, —NH—, or —NHC(O)—;
Ring C is optional, and when present is 4- to 12-membered heterocyclyl optionally substituted with one or more R^{C};
   Linker is a bond, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein
      * indicates the point of attachment to Ring C when Ring C is present, and * indicates the point of attachment to W when Ring C is absent; and
      ** indicates the point of attachment to U when Ring 2 is present, and ** indicates the point of attachment to V when Ring 2 is absent;
      L¹ is a bond or C₁₋₆ alkylene;
      L² is a 3- to 10-membered cycloalkyl or 4- to 12-membered heterocyclyl optionally substituted with one or more R^{L}; and
      L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y});
   U is absent when Ring 2 is absent, and is a bond or C(O) when Ring 2 is present;
   Ring 2 is optional, and when present is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b};
each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{2a}, R^{3a}, R^{4a}, and R^{5a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{2b}, R^{3b}, R^{4b}, and R^{5b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R^{C} is independently halo, OH, C₁₋₄alkyl, or C₁₋₄haloalkyl;
each R^{L} is independently halo, OH, C₁₋₄alkyl, or C₁₋₄haloalkyl;
each R^{Q} is independently H, halo, OH, C₁₋₄alkyl, or C₁₋₄haloalkyl;
each R^{V} is independently halo, OH, C₁₋₄alkyl, or C₁₋₄haloalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, or C₁₋₄alkylene-S(O)ₙ—C₁₋₄alkyl; and
each n is independently 0, 1, or 2;
   wherein:
(a) Ring 1 comprises an intraannular amide, or
(b) V comprises an amide, or
(c) V is taken together with an atom of Ring 1, Ring 2, or Ring 5 to which it is attached, to form an amide;
   and wherein:
when Ring 3 is 5-membered heteroaryl, Ring 4 is present and is not cycloalkyl or tetrahydropyranyl.

In one aspect, provided is a compound of Formula (I):

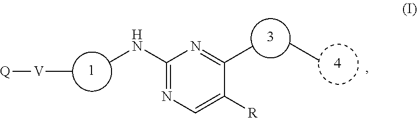

or a tautomer or stereoisomer thereof, or a pharmaceutically acceptable salt of any of the foregoing, wherein:
R is halo;
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Ring 3 is a monocyclic ring selected from the group consisting of 3- to 6-membered cycloalkyl optionally substituted with one or more R^{3a}; 4- to 6-membered heterocyclyl optionally substituted with one or more R^{3a}; phenyl optionally substituted with one or more R^{3b}; or 5- to 6-membered heteroaryl optionally substituted with one or more R^{3b};
Ring 1 is a 4- to 8-membered cycloalkyl, saturated 4- to 8-membered heterocyclyl comprising at least one annular ether or annular amine, or saturated 4- to 8-membered heterocyclyl comprising an annular amide, each of which is optionally substituted with one or more R¹;
V is a bond, —C₁₋₄alkylene-, —C(O)—, —C₁₋₄alkylene-C(O)-#, —C(O)N(R^{V})-#, —N(R^{V})C(O)-#, —C₁₋₄alkylene-C(O)N(R^{V})-# or —C₁₋₄alkylene-N(R^{V})C(O)-#, wherein # indicates the point of attachment to Ring 1; and
Q is R^{Q}, or Ring 5,
wherein Ring 5 is selected from the group consisting of 3- to 10-membered cycloalkyl optionally substituted with one or more R^{5a}, 4- to 12-membered heterocyclyl optionally substituted with one or more R^{5a}, and 5- to 12-membered heteroaryl optionally substituted with one or more R^{5b}
or
Q is of Formula (i)

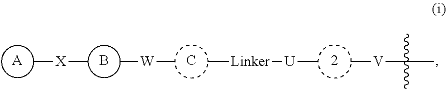

wherein:
Ring A is selected from the group consisting of

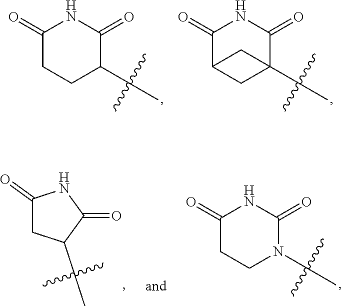 [SMILES: *C1CCC(=O)NC1=O ; *C12CC(C1)C(=O)NC2=O ; *C1CC(=O)NC1=O ; *N1CCC(=O)NC1=O]

each of which is optionally substituted with one or more C₁₋₄alkyl or halo;
X is a bond, —O—, —NH—, or —NHC(O)—;
Ring B is selected from the group consisting of

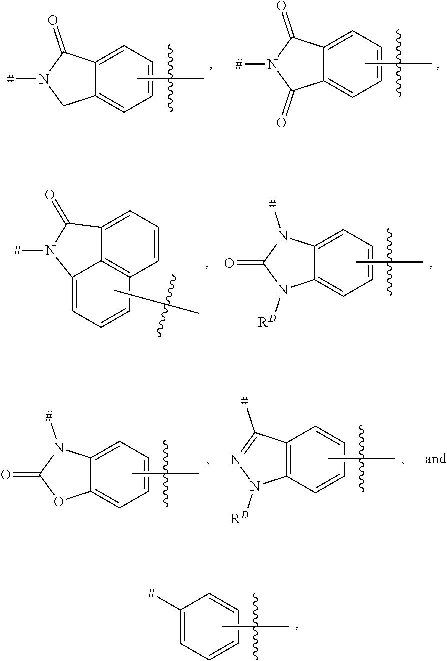

each of which is optionally substituted with one or more halo, C₁₋₄alkyl, C₁₋₄haloalkyl, or OH, and wherein R^{D} is H or C₁₋₄alkyl, wherein # indicates attachment to X;
W is a bond, —O—, —NH—, or —NHC(O)—;
Ring C is optional, and when present is 4- to 12-membered heterocyclyl optionally substituted with one or more R^{C};
   Linker is a bond, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein
      * indicates the point of attachment to Ring C when Ring C is present, and * indicates the point of attachment to W when Ring C is absent; and
      ** indicates the point of attachment to U when Ring 2 is present, and ** indicates the point of attachment to V when Ring 2 is absent;
      L¹ is a bond or C₁₋₆ alkylene;
      L² is a 3- to 10-membered cycloalkyl or 4- to 12-membered heterocyclyl optionally substituted with one or more R^{L}; and
      L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y});
   U is absent when Ring 2 is absent, and is a bond or C(O) when Ring 2 is present;
   Ring 2 is optional, and when present is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b};
each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)₁—C₁₋₄alkyl;
each R^{2a}, R^{3a}, R^{4a}, and R^{5a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{2b}, R^{3b}, R^{4b}, and R^{5b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R^{C} is independently halo, OH, C₁₋₄alkyl, or C₁₋₄haloalkyl;
each R^{L} is independently halo, OH, C₁₋₄alkyl, or C₁₋₄haloalkyl;
each R^{Q} is independently H, halo, OH, C₁₋₄alkyl, or C₁₋₄haloalkyl;
each R^{V} is independently halo, OH, C₁₋₄alkyl, or C₁₋₄haloalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ-C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, or C₁₋₄alkylene-S(O)ₙ-C₁₋₄alkyl; and
each n is independently 0, 1, or 2;
   wherein:
(i) when Ring 1 is a 4- to 8-membered heterocyclyl ring comprising at least one annular amine, optionally substituted with one or more R¹, then V is attached to an annular nitrogen atom of Ring 1 and is —C(O)— or —C₁₋₄alkylene-C(O)-#; and
(ii) when Ring 1 is a 4- to 8-membered heterocyclyl ring comprising at least one annular amide, optionally substituted with one or more R¹, then V is attached to an annular nitrogen atom of Ring 1 and is a bond or —C₁₋₄alkylene-; and
(iii) when Ring 1 is C₄₋₈ cycloalkyl or a 4- to 8-membered heterocyclyl ring comprising no annular amine or annular amide, each of which is optionally substituted with one or more R¹, then:
   (a) V is —C(O)N(R^{V})-#, —N(R^{V})C(O)-#, —C₁₋₄alkylene-C(O)N(R^{V})-# or —C₁₋₄alkylene-N(R^{V})C(O)-#; or
   (b) Ring 5 is 4- to 12-membered heterocyclyl optionally substituted with one or more R^{5a}, and V is —C(O)— and is attached to an annular nitrogen atom of Ring 5; or
   (c) Ring 2 is 4- to 12-membered heterocyclyl optionally substituted with one or more R^{2a}, and V is —C(O)— and is attached to an annular nitrogen atom of Ring 2;
      and wherein:
when Ring 3 is 5-membered heteroaryl, Ring 4 is present and is not cycloalkyl or tetrahydropyranyl.

In one aspect, provided is a compound of Formula (I):

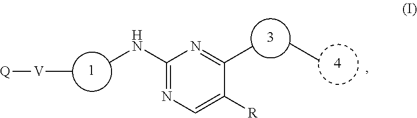

or a tautomer or stereoisomer thereof, or a pharmaceutically acceptable salt of any of the foregoing, wherein:
R is halo;
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Ring 3 is a monocyclic ring selected from the group consisting of 4- to 6-membered heterocyclyl optionally substituted with one or more R^{3a}; phenyl optionally substituted with one or more R^{3b}; or 5- to 6-membered heteroaryl optionally substituted with one or more R^{3b};
Ring 1 is a 4- to 8-membered cycloalkyl or saturated 4- to 8-membered heterocyclyl, each of which is optionally substituted with one or more R¹, wherein each 4- to 8-membered heterocyclyl of Ring 1 comprises at least one N heteroatom;
V is a —C(O)—, —C₁₋₄alkylene-C(O)-#, —C(O)N(R^{V})-#, —N(R′)C(O)-#, or —C₁₋₄alkylene-C(O)N(R^{V})-#, wherein # indicates the point of attachment to Ring 1; and
Q is R^{Q},
or
Q is of Formula (i)

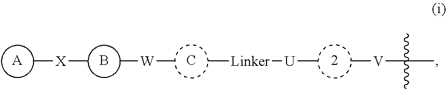

wherein:
Ring A is

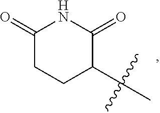 [SMILES: *C1CCC(=O)NC1=O]

is optionally substituted with one or more C₁₋₄alkyl or halo;
X is a bond, or —NH—;
Ring B is selected from the group consisting of

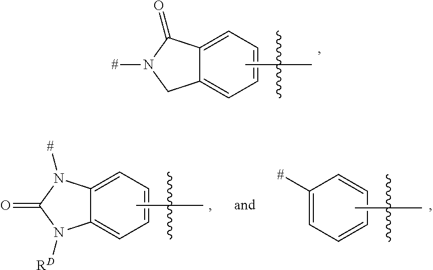

each of which is optionally substituted with one or more halo, C₁₋₄alkyl, C₁₋₄haloalkyl, or OH, and wherein R^{D} is H or C₁₋₄alkyl, wherein # indicates attachment to X;
W is a bond or —O—;
Ring C is optional, and when present is 6-membered heterocyclyl optionally substituted with one or more R^{C};
   Linker is a bond, C₁₋₁₀alkylene, or C(O)C₁₋₁₀alkylene;
   U is absent when Ring 2 is absent, and is a bond when Ring 2 is present;
   Ring 2 is optional, and when present is 6-membered or 11-membered heterocyclyl optionally substituted with one or more R^{2a};
each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ-C₁₋₄alkyl;
each R^{2a}, R^{3a}, and R^{4a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{3b} and R^{4b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R^{C} is independently halo, OH, C₁₋₄alkyl, or C₁₋₄haloalkyl;
each R^{Q} is independently H, halo, OH, C₁₋₄alkyl, or C₁₋₄haloalkyl;
each R^{V} is independently halo, OH, C₁₋₄alkyl, or C₁₋₄haloalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O), —C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, or C₁₋₄alkylene-S(O)ₙ-C₁₋₄alkyl; and
each n is independently 0, 1, or 2;
   wherein:
(i) when Ring 1 is a 4- to 8-membered heterocyclyl ring comprising at least one annular amine, optionally substituted with one or more R¹, then V is attached to an annular nitrogen atom of Ring 1 and is —C(O)— or —C₁₋₄alkylene-C(O)-#; and
(ii) when Ring 1 is a 4- to 8-membered heterocyclyl ring comprising at an annular amide, optionally substituted with one or more R¹, then V is attached to the annular amide nitrogen atom of Ring 1 and is a bond or —C₁₋₄alkylene-; and
(iii) when Ring 1 is C₄₋₈ cycloalkyl, optionally substituted with one or more R¹, then:
   (a) V is —C(O)N(R^{V})-# or —N(R^{V})C(O)-#; or
   (b) Ring 2 is 6- or 11-membered heterocyclyl optionally substituted with one
or more R^{2a}, and V is —C(O)— and is attached to an annular nitrogen atom of Ring 2;
   and wherein:
when Ring 3 is 5-membered heteroaryl, Ring 4 is present and is not cycloalkyl or tetrahydropyranyl.

In some embodiments of the compound of Formula (I), R is chloro, fluoro, or bromo. In some embodiments of the compound of Formula (I), R is chloro or fluoro. In some embodiments of the compound of Formula (I), R is chloro. In some embodiments of the compound of Formula (I), R is fluoro.

In some embodiments of the compound of Formula (I), Ring 1 is a C₄₋₈ cycloalkyl optionally substituted with one or more R¹, wherein each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ-C₁₋₄alkyl. In some embodiments, Ring 1 is a C₅₋₈ cycloalkyl optionally substituted with one or more R¹. In some embodiments, Ring 1 is a C₆₋₈ cycloalkyl optionally substituted with one or more R¹. In some embodiments, Ring 1 is cyclohexyl or spiro[3.3.]heptanyl, each of which is optionally substituted with one or more R¹. In some embodiments, Ring 1 is

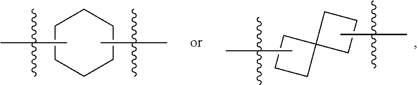

each of which is optionally substituted with one or more R¹. In some embodiments, Ring 1 is

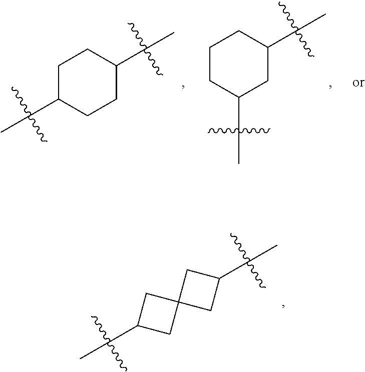 [SMILES: *C1CCC(*)CC1 ; *C1CCCC(*)C1 ; *C1CC2(C1)CC(*)C2]

each of which is optionally substituted with one or more R¹. In some embodiments, Ring 1 is

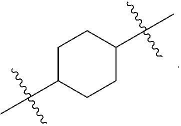 [SMILES: *C1CCC(*)CC1]

In some embodiments, Ring 1 is

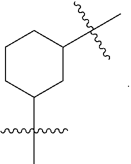 [SMILES: *C1CCCC(*)C1]

In some embodiments, Ring 1 is

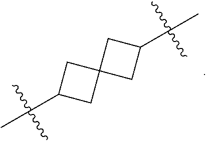 [SMILES: *C1CC2(C1)CC(*)C2]

In some embodiments, Ring 1 is

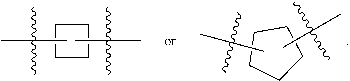

In some embodiments, Ring 1 is

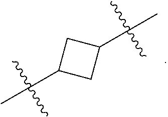 [SMILES: *C1CC(*)C1]

In some embodiments, Ring 1 is

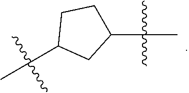 [SMILES: *C1CCC(*)C1]

In some embodiments of the compound of Formula (I), Ring 1 is a saturated 4- to 8-membered heterocyclyl comprising at least one annular ether or annular amine, optionally substituted with one or more R¹, wherein each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ-C₁₋₄alkyl. In some embodiments of the compound of Formula (I), Ring 1 is a saturated 5- to 8-membered heterocyclyl comprising at least one annular ether or annular amine, optionally substituted with one or more R¹. In some embodiments of the compound of Formula (I), Ring 1 is a saturated 6- to 8-membered heterocyclyl comprising at least one annular ether or annular amine, optionally substituted with one or more R¹. In some embodiments, Ring 1 is piperidinyl or 2-azaspiro[3.3]heptanyl, each of which is optionally substituted with one or more R¹. In some embodiments, Ring 1 is

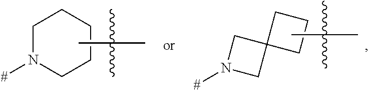

wherein # indicates attachment to V, each of which is optionally substituted with one or more R¹. In some embodiments, Ring 1 is

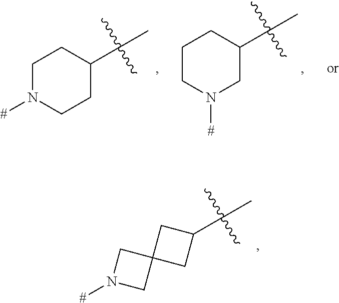

wherein # indicates attachment to V, each of which is optionally substituted with one or more R¹. In some embodiments, Ring 1 is

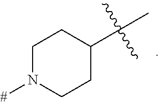

In some embodiments, Ring 1 is

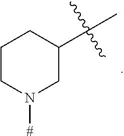

In some embodiments, Ring 1 is

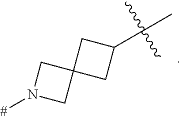

In some embodiments of the compound of Formula (I), Ring 1 is a saturated 4- to 8-membered heterocyclyl comprising an annular amide, optionally substituted with one or more R¹, wherein each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl. In some embodiments, Ring 1 is

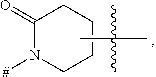

optionally substituted with one or more R¹, wherein # indicates attachment to V. In some embodiments, Ring 1 is

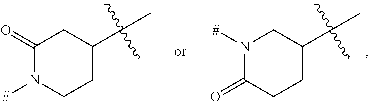

each of which is optionally substituted with one or more R¹. In some embodiments, Ring 1 is

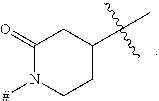

In some embodiments, Ring 1 is

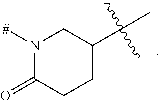

In some embodiments of the compound of Formula (I), Ring 1 is a C₄₋₈ cycloalkyl optionally substituted with one or more R¹, wherein each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl; and V is —C(O)N(R^{V})-##, —N(R^{V})C(O)-##, —C₁₋₄alkylene-C(O)N(R^{V})-## or —C₁₋₄alkylene-N(R^{V})C(O)-##, wherein ## indicates attachment to Ring 1. In some embodiments, Ring 1 is a C₅₋₈ cycloalkyl optionally substituted with one or more R¹, and V is —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is a C₆₋₈ cycloalkyl optionally substituted with one or more R¹, and V is —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is cyclohexyl or spiro[3.3.]heptanyl, each of which is optionally substituted with one or more R¹, and V is —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is

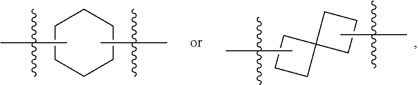

each of which is optionally substituted with one or more R¹, and V is —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is

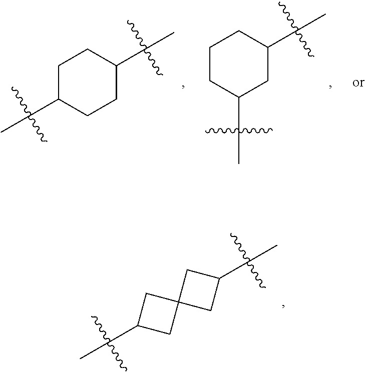 [SMILES: *C1CCC(*)CC1 ; *C1CCCC(*)C1 ; *C1CC2(C1)CC(*)C2]

each of which is optionally substituted with one or more R¹, and V is —C(O)N(R^{V})-##, —N(R^{V})C(O)-##, —C₁₋₄ alkylene-C(O)N(R^{V})-## or —C₁₋₄alkylene-N(R^{V})C(O)-##. In some embodiments, Ring 1 is

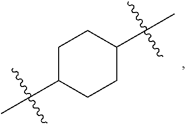 [SMILES: *C1CCC(*)CC1]

and V is —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is

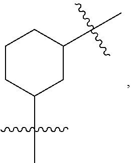 [SMILES: *C1CCCC(*)C1]

and V is —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is

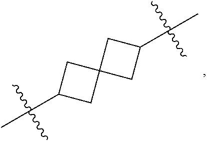 [SMILES: *C1CC2(C1)CC(*)C2]

and V is —C(O)N(R^{V})-##, —N(R^{V})C(O)-##, —C₁₋₄alkylene-C(O)N(R^{V})-## or —C₁₋₄alkylene-N(R^{V})C(O)-##.

In some embodiments of the compound of Formula (I), Ring 1 is a C₄₋₈ cycloalkyl optionally substituted with one or more R¹, wherein each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl; and V is —OC(O)N(R^{V})-##, —N(R^{V})—C(O)O##, —C(O)N(R^{V})-##, —N(R^{V})C(O)-##, —C₁₋₄alkylene-C(O)N(R^{V})-## or —C₁₋₄alkylene-N(R^{V})C(O)-##, wherein ## indicates attachment to Ring 1. In some embodiments, Ring 1 is a C₅₋₈ cycloalkyl optionally substituted with one or more R¹, and V is —OC(O)N(R^{V})-##, —N(R^{V})—C(O)O##, —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is a C₆₋₈ cycloalkyl optionally substituted with one or more R¹, and V is —OC(O)N(R^{V})-##, —N(R^{V})—C(O)O##, —C(O)N(R^{V})-##, —N(R^{V})C(O)-##, —C₁₋₄alkylene-C(O)N(R^{V})-## or —C₁₋₄ alkylene-N(R^{V})C(O)-##. In some embodiments, Ring 1 is cyclohexyl or spiro[3.3.]heptanyl, each of which is optionally substituted with one or more R¹, and V is —OC(O)N(R^{V})-##, —N(R^{V})—C(O)O##, —C(O)N(R^{V})-##, —N(R^{V})C(O)-##, —C₁₋₄alkylene-C(O)N(R^{V})-## or —C₁₋₄ alkylene-N(R^{V})C(O)-##. In some embodiments, Ring 1 is

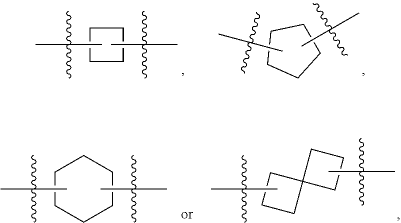

each of which is optionally substituted with one or more R¹, and V is —OC(O)N(R^{V})-##, —N(R^{V})—C(O)O##, —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is

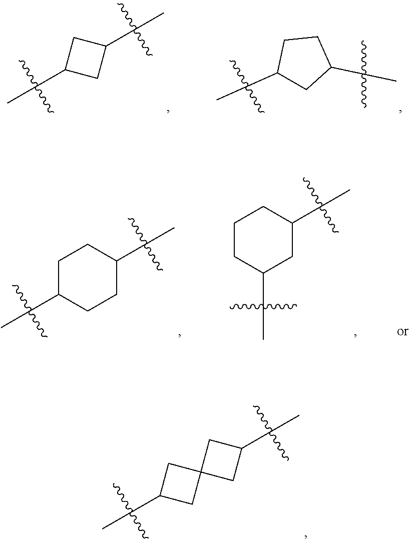 [SMILES: *C1CC(*)C1 ; *C1CCC(*)C1 ; *C1CCC(*)CC1 ; *C1CCCC(*)C1 ; *C1CC2(C1)CC(*)C2]

each of which is optionally substituted with one or more R¹, and V is —OC(O)N(R^{V})-##, —N(R^{V})—C(O)O##, —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is

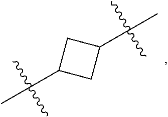 [SMILES: *C1CC(*)C1]

and V is —OC(O)N(R^{V})-##, —N(R^{V})—C(O)O##, —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is

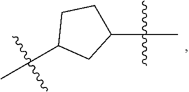 [SMILES: *C1CCC(*)C1]

and V is —C(O)N(R)-##, —N(R^{V})—C(O)O##, —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is

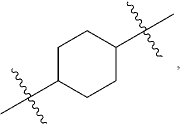 [SMILES: *C1CCC(*)CC1]

and V is —OC(O)N(R^{V})-##, —N(R^{V})—C(O)O##, —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is

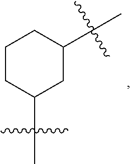 [SMILES: *C1CCCC(*)C1]

and V is —OC(O)N(R^{V})-##, —N(R^{V})—C(O)O##, —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is

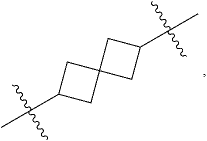 [SMILES: *C1CC2(C1)CC(*)C2]

and V is —OC(O)N(R^{V})-##, —N(R^{V})—C(O)O##, —C(O)N(R^{V})-##, —N(R^{V})C(O)-##, —C₁₋₄alkylene-C(O)N(R^{V})-## or —C₁₋₄alkylene-N(R^{V})C(O)-##.

In some embodiments of the compound of Formula (I), Ring 1 is a 4- to 8-membered heterocyclyl ring comprising at least one annular ether and no annular amine or annular amide, optionally substituted with one or more R¹, wherein each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ-C₁₋₄alkyl; and V is —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is a 5- to 8-membered heterocyclyl ring comprising no annular amine or annular amide, optionally substituted with one or more R¹, and V is —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is a 6- to 8-membered heterocyclyl ring comprising no annular amine or annular amide, optionally substituted with one or more R¹, and V is —C(O)N(R^{V})-##, —N(R^{V})C(O)-##, —C₁₋₄alkylene-C(O)N(R^{V})-## or —C₁₋₄alkylene-N(R^{V})C(O)-##.

In some embodiments of the compound of Formula (I), Ring 1 is a 4- to 8-membered heterocyclyl ring comprising at least one annular ether and no annular amine or annular amide, optionally substituted with one or more R¹, wherein each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O), —C₁₋₄alkyl; and V is —OC(O)N(R^{V})-##, —N(R^{V})—C(O)O##, —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is a 5- to 8-membered heterocyclyl ring comprising no annular amine or annular amide, optionally substituted with one or more R¹, and V is —OC(O)N(R^{V})-##, —N(R^{V})—C(O)O##, —C(O)N(R^{V})-##, —N(R^{V})C(O)-##, —C₁₋₄alkylene-C(O)N(R^{V})-## or —C₁₋₄alkylene-N(R^{V})C(O)-##. In some embodiments, Ring 1 is a 6- to 8-membered heterocyclyl ring comprising no annular amine or annular amide, optionally substituted with one or more R¹, and V is —OC(O)N(R^{V})-##, —N(R^{V})—C(O)O##, —C(O)N(R^{V})-##, —N(R^{V})C(O)-##, —C₁₋₄alkylene-C(O)N(R^{V})-## or —C₁₋₄alkylene-N(R^{V})C(O)-##.

In some embodiments of the compound of Formula (I), Ring 1 is a saturated 4- to 8-membered heterocyclyl comprising at least one annular amine and no annular amide, optionally substituted with one or more R¹, wherein each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ-C₁₋₄alkyl; and V is —C(O)-## or —C₁₋₄alkylene-C(O)-##, wherein ## indicates attachment to Ring 1. In some embodiments of the compound of Formula (I), Ring 1 is a saturated 5- to 8-membered heterocyclyl comprising at least one annular amine, optionally substituted with one or more R¹, and V is —C(O)-## or —C₁₋₄alkylene-C(O)-##, wherein ## indicates attachment to Ring 1. In some embodiments of the compound of Formula (I), Ring 1 is a saturated 6- to 8-membered heterocyclyl comprising at least one or annular amine, optionally substituted with one or more R¹, and V is —C(O)-## or —C₁₋₄alkylene-C(O)-##, wherein ## indicates attachment to Ring 1. In some embodiments, Ring 1 is piperidinyl or 2-azaspiro[3.3]heptanyl, each of which is optionally substituted with one or more R¹, and V is —C(O)-## or —C₁₋₄alkylene-C(O)-##, wherein ## indicates attachment to Ring 1. In some embodiments, Ring 1 is

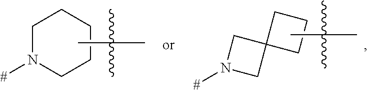

each of which is optionally substituted with one or more R¹, wherein # indicates attachment to V; and V is —C(O)-## or —C₁₋₄alkylene-C(O)-##. In some embodiments, Ring 1 is

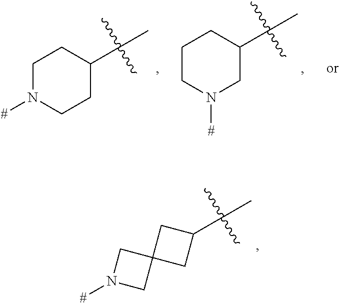

each of which is optionally substituted with one or more R¹, and V is —C(O)-## or —C₁₋₄alkylene-C(O)-##. In some embodiments, Ring 1 is

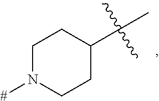

and V is —C(O)-## or —C₁₋₄alkylene-C(O). In some embodiments, Ring 1 is

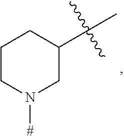

and V is —C(O)-## or —C₁₋₄alkylene-C(O). In some embodiments, Ring 1 is

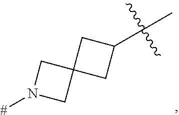

### and V is —C(O)-## or —C₁₋₄alkylene-C(O).

In some embodiments of the compound of Formula (I), Ring 1 is a saturated 4- to 8-membered heterocyclyl comprising at least one annular amine and no annular amide, optionally substituted with one or more R¹, wherein each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)₁—C₁₋₄alkyl; and V is —C(O)O-##, —OC(O)##, —C(O)-## or —C₁₋₄alkylene-C(O)-##, wherein ## indicates attachment to Ring 1. In some embodiments of the compound of Formula (I), Ring 1 is a saturated 5- to 8-membered heterocyclyl comprising at least one annular amine, optionally substituted with one or more R¹, and V is —C(O)O-##, —OC(O)##, —C(O)-## or —C₁₋₄alkylene-C(O)-##, wherein ## indicates attachment to Ring 1. In some embodiments of the compound of Formula (I), Ring 1 is a saturated 6- to 8-membered heterocyclyl comprising at least one or annular amine, optionally substituted with one or more R¹, and V is —C(O)O-##, —OC(O)##, —C(O)-## or —C₁₋₄alkylene-C(O)-##, wherein ## indicates attachment to Ring 1. In some embodiments, Ring 1 is piperidinyl or 2-azaspiro[3.3]heptanyl, each of which is optionally substituted with one or more R¹, and V is —C(O)O-##, —OC(O)##, —C(O)-## or —C₁₋₄alkylene-C(O)-##, wherein ## indicates attachment to Ring 1. In some embodiments, Ring 1 is

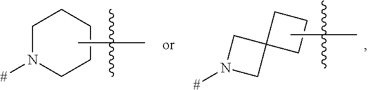

each of which is optionally substituted with one or more R¹, wherein # indicates attachment to V; and V is —OC(O)##, —C(O)-## or —C₁₋₄alkylene-C(O)-##. In some embodiments, Ring 1 is

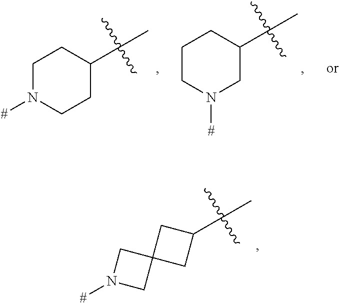

each of which is optionally substituted with one or more R¹, and V is —OC(O)##, —C(O)-## or —C₁₋₄alkylene-C(O)-##. In some embodiments, Ring 1 is

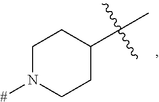

and V is —OC(O)##, —C(O)-## or —C₁₋₄alkylene-C(O). In some embodiments, Ring 1 is

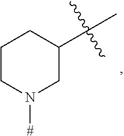

and V is —OC(O)##, —C(O)-## or —C₁₋₄alkylene-C(O). In some embodiments, Ring 1 is

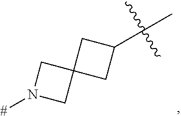

### and V is —OC(O)##, —C(O)-## or —C₁₋₄alkylene-C(O).

In some embodiments of the compound of Formula (I), Ring 1 is a saturated 4- to 8-membered heterocyclyl comprising an annular amide, optionally substituted with one or more R¹, and V is a bond or —C₁₋₄alkylene-. In some embodiments, Ring 1 is

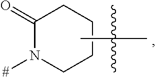

optionally substituted with one or more R¹, wherein # indicates attachment to V; and V is a bond or —C₁₋₄alkylene-. In some embodiments, Ring 1 is

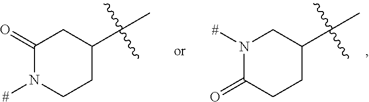

each of which is optionally substituted with one or more R¹; and V is a bond or —C₁₋₄alkylene-. In some embodiments, Ring 1 is

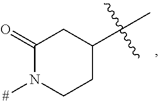

and V is a bond or —C₁₋₄alkylene-. In some embodiments, Ring 1 is

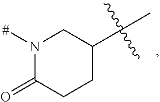

and V is a bond or —C₁₋₄alkylene-.

In some embodiments, Ring 1 comprises an intraannular amide, or V comprises an amide, or V is taken together with an atom of Ring 1, Ring 2, or Ring 5 to which it is attached, to form an amide. In some embodiments, Ring 1 comprises an intraannular amide. In some embodiments, V comprises an amide. In some embodiments, V is taken together with an atom of Ring 1, Ring 2, or Ring 5 to which it is attached, to form an amide. In some embodiments of Formula (I), (II), (III), or (IV), or a related formula as appropriate, V is taken together with an atom of Ring 1 to which it is attached, to form an amide. In some embodiments of Formula (I) or (II), or a related formula as appropriate, V is taken together with an atom of Ring 5 to which it is attached, to form an amide. In some embodiments of Formula (I) or (IV), or a related formula as appropriate, V is taken together with an atom of Ring 5 to which it is attached, to form an amide.

In some embodiments, V comprises a carbamate, or V is taken together with an atom of Ring 1, Ring 2, or Ring 5 to which it is attached, to form a carbamate. In some embodiments, V comprises a carbamate. In some embodiments, V is taken together with an atom of Ring 1, Ring 2, or Ring 5 to which it is attached, to form a carbamate. In some embodiments of Formula (I′), (I), (II), (III), or (IV), or a related formula as appropriate, V is taken together with an atom of Ring 1 to which it is attached, to form a carbamate. In some embodiments of Formula (I′), (I), or (II), or a related formula as appropriate, V is taken together with an atom of Ring 5 to which it is attached, to form a carbamate. In some embodiments of Formula (I′), (I), or (IV), or a related formula as appropriate, V is taken together with an atom of Ring 5 to which it is attached, to form a carbamate.

In some embodiments, when Ring 1 is a 4- to 8-membered heterocyclyl ring comprising at least one annular amine, optionally substituted with one or more R¹, then V is attached to an annular nitrogen atom of Ring 1 and is —C(O)— or —C₁₋₄alkylene-C(O)-#.

In some embodiments, when Ring 1 is a 4- to 8-membered heterocyclyl ring comprising at least one annular amine, optionally substituted with one or more R¹, then V is attached to an annular nitrogen atom of Ring 1 and is —OC(O)##.

In some embodiments, when Ring 1 is a 4- to 8-membered heterocyclyl ring comprising at least one annular amide, optionally substituted with one or more R¹, then V is attached to an annular nitrogen atom of Ring 1 and is a bond or —C₁₋₄alkylene-.

In some embodiments, when Ring 1 is C₄₋₈ cycloalkyl or a 4- to 8-membered heterocyclyl ring comprising no annular amine or annular amide, each of which is optionally substituted with one or more R¹, then (a) V is —C(O)N(R^{V})-#, —N(R^{y})C(O)-#, —C₁₋₄alkylene-C(O)N(R^{V})-# or —C₁₋₄alkylene-N(R^{V})C(O)-#; or (b) Ring 5 is 4- to 12-membered heterocyclyl optionally substituted with one or more R^{5a}, and V is —C(O)— and is attached to an annular nitrogen atom of Ring 5; or (c) Ring 2 is 4- to 12-membered heterocyclyl optionally substituted with one or more R^{2a}, and V is —C(O)— and is attached to an annular nitrogen atom of Ring 2.

In some embodiments, when Ring 1 is C₄₋₈ cycloalkyl or a 4- to 8-membered heterocyclyl ring comprising no annular amine or annular amide, each of which is optionally substituted with one or more R¹, then (a) V is —OC(O)N(R^{V})-# or —N(R^{V})—C(O)O #, wherein # indicates attachment to Ring 1; or (b) Ring 5 is 4- to 12-membered heterocyclyl optionally substituted with one or more R^{5a}, and V is —C(O)O-# and is attached to an annular nitrogen atom of Ring 5, wherein # indicates attachment to Ring 1; or (c) Ring 2 is 4- to 12-membered heterocyclyl optionally substituted with one or more R^{2a}, and V is —C(O)O-# and is attached to an annular nitrogen atom of Ring 2, wherein # indicates attachment to Ring 1.

In some embodiments, when Ring 1 is C₄₋₈ cycloalkyl or a 4- to 8-membered heterocyclyl ring comprising no annular amine or annular amide, each of which is optionally substituted with one or more R¹, then V is —C(O)N(R^{V})-#, —N(R^{V})C(O)-#, —C₁₋₄alkylene-C(O)N(R^{V})-# or —C₁₋₄alkylene-N(R^{V})C(O)-#.

In some embodiments, when Ring 1 is C₄₋₈ cycloalkyl or a 4- to 8-membered heterocyclyl ring comprising no annular amine or annular amide, each of which is optionally substituted with one or more R¹, then V is —OC(O)N(R^{V})-# or —N(R^{V})—C(O)O #, wherein # indicates attachment to Ring 1.

In some embodiments, when Ring 1 is C₄₋₈ cycloalkyl or a 4- to 8-membered heterocyclyl ring comprising no annular amine or annular amide, each of which is optionally substituted with one or more R¹, then Ring 5 is 4- to 12-membered heterocyclyl optionally substituted with one or more R^{5a}, and V is —C(O)— and is attached to an annular nitrogen atom of Ring 5.

In some embodiments, when Ring 1 is C₄₋₈ cycloalkyl or a 4- to 8-membered heterocyclyl ring comprising no annular amine or annular amide, each of which is optionally substituted with one or more R¹, then Ring 5 is 4- to 12-membered heterocyclyl optionally substituted with one or more R^{5a}, and V is —C(O)O-# and is attached to an annular nitrogen atom of Ring 5, wherein # indicates attachment to Ring 1.

In some embodiments, when Ring 1 is C₄₋₈ cycloalkyl or a 4- to 8-membered heterocyclyl ring comprising no annular amine or annular amide, each of which is optionally substituted with one or more R¹, then Ring 2 is 4- to 12-membered heterocyclyl optionally substituted with one or more R^{2a}, and V is —C(O)— and is attached to an annular nitrogen atom of Ring 2.

In some embodiments, when Ring 1 is C₄₋₈ cycloalkyl or a 4- to 8-membered heterocyclyl ring comprising no annular amine or annular amide, each of which is optionally substituted with one or more R¹, then Ring 2 is 4- to 12-membered heterocyclyl optionally substituted with one or more R^{2a}, and V is —C(O)O-# and is attached to an annular nitrogen atom of Ring 2, wherein # indicates attachment to Ring 1.

In some embodiments of the compound of Formula (I), the compound is of Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w):

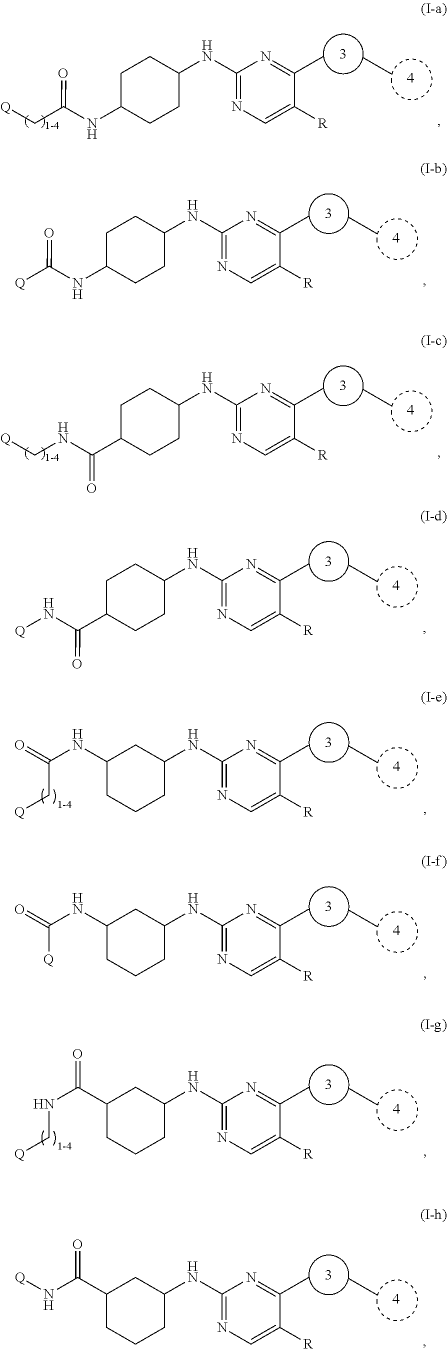

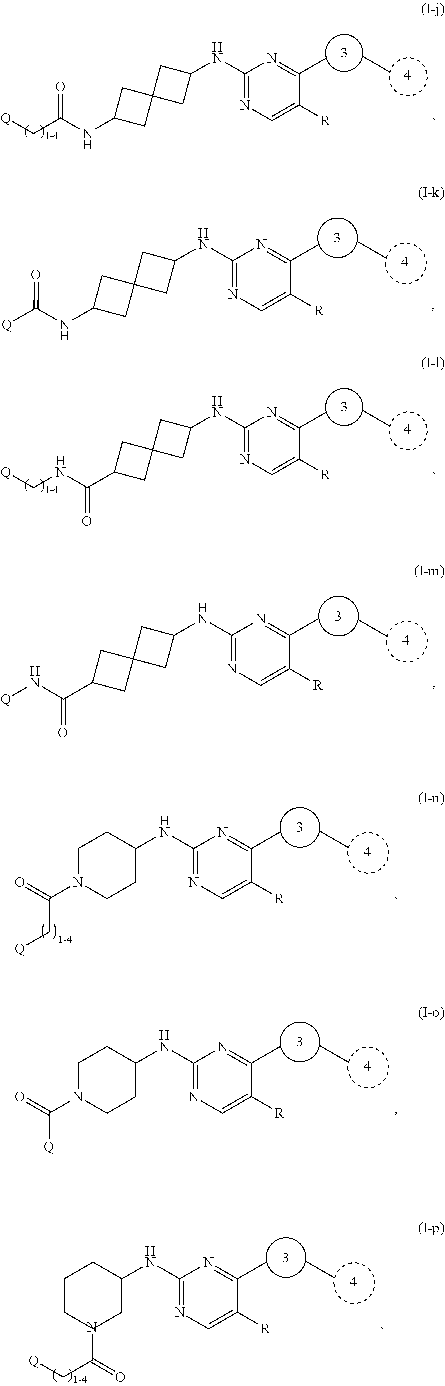

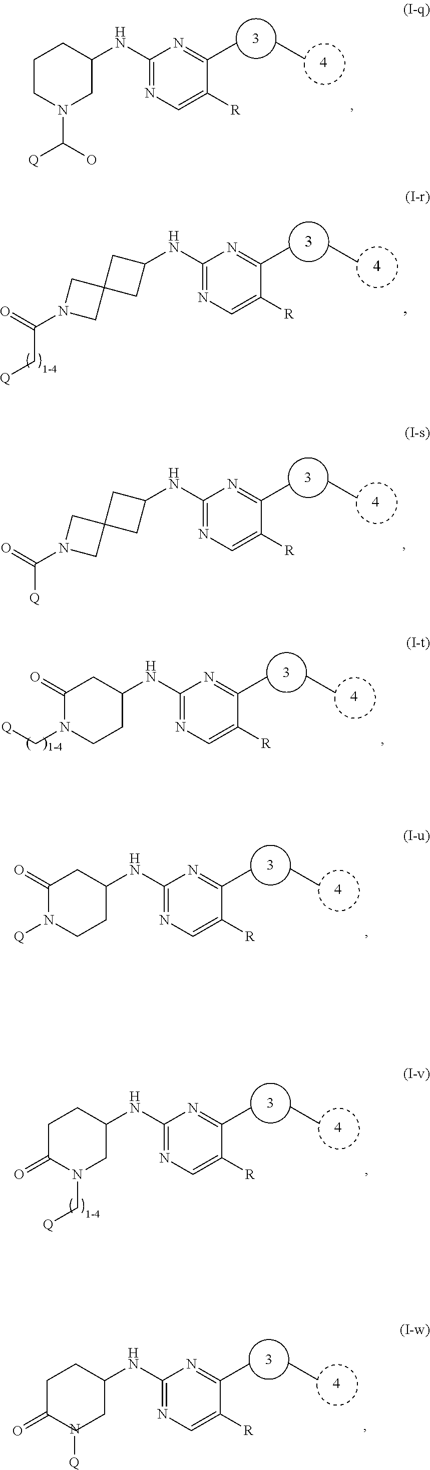

wherein Q, R, Ring 3, and Ring 4 are as defined herein for Formula (I). In some embodiments, Ring 4 is present.

In some embodiments of the compound of Formula (I), the compound is of Formula (I-z), (I-aa), (I-ab), (I-ac), (I-ad), (I-ae), (I-af), (I-ag):

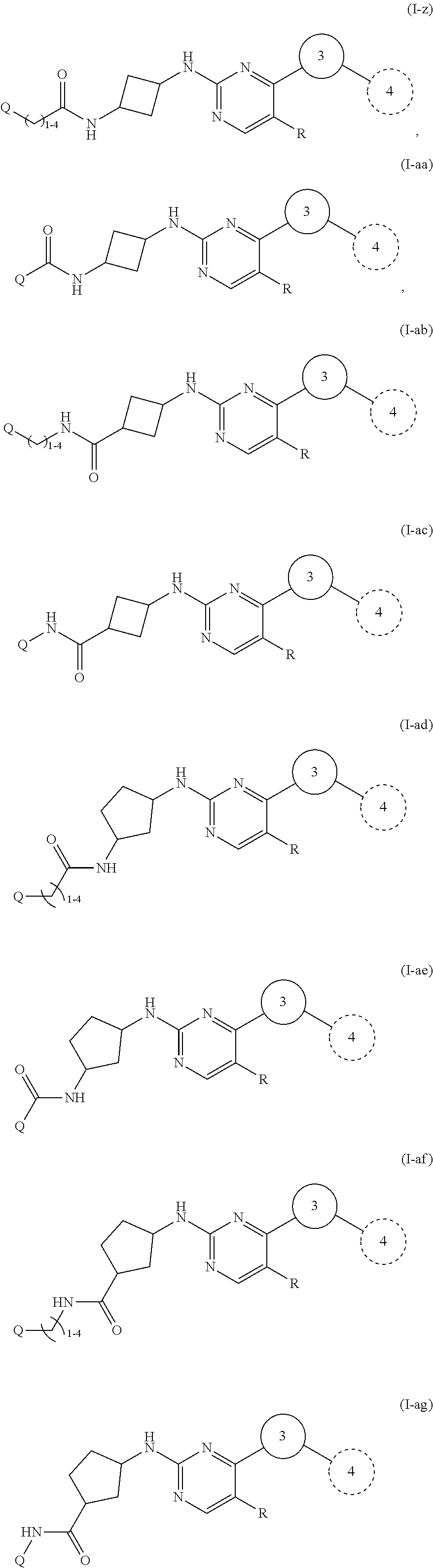

wherein Q, R, Ring 3, and Ring 4 are as defined herein for Formula (I). In some embodiments, Ring 4 is present.

In some embodiments of the compound of Formula (I), or a related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), Ring 3 is phenyl optionally substituted with one or more R^{3b}, and Ring 4 is phenyl optionally substituted with one or more R^{4b}. In some embodiments, the compound is of Formula (I-x),

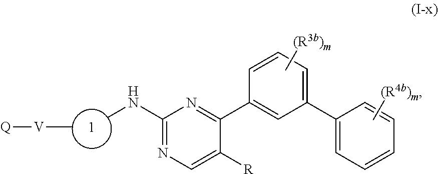

or a tautomer or stereoisomer thereof, or a pharmaceutically acceptable salt of any of the foregoing, wherein each m is independently an integer from 0 to 4, and Q, V, R, and Ring 1 are as described herein for Formula (I). In some embodiments, each m is independently an integer from 0 to 2. In some embodiments, each m is 0 or 1. In some embodiments, Ring 3 is phenyl, Ring 4 is phenyl, and the compound is of Formula (I-y)

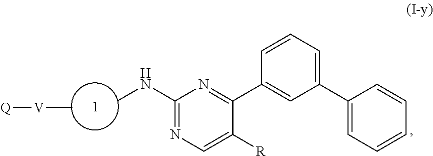

or a tautomer or stereoisomer thereof, or a pharmaceutically acceptable salt of any of the foregoing, wherein Q, V, R, and Ring 1 are as described herein for Formula (I). In some embodiments of Formula (I-x) or (I-y), Ring 1 is cyclohexyl or piperidinyl, each of which is optionally substituted with one or more R¹. In some embodiments, Ring 1 is cyclohexyl or piperidinyl, each of which is optionally substituted with one or more R¹, and Q is of Formula (i). In some embodiments, Ring 1 is cyclohexyl or piperidinyl, each of which is optionally substituted with one or more R¹, and Q is R^{Q}.

In some embodiments, Ring 3 is 5-membered heteroaryl, and Ring 4 is present. In some embodiments, Ring 3 is 5-membered heteroaryl, and Ring 4 is not cycloalkyl or tetrahydropyranyl. In some embodiments, Ring 3 is 5-membered heteroaryl, and Ring 4 is phenyl optionally substituted with one or more R^{4b}, 5- to 6-membered heteroaryl optionally substituted with one or more R^{4b}, or 4- to 6-membered heterocyclyl optionally substituted with one or more R^{4a}, wherein Ring 4 is not tetrahydropyranyl. In some embodiments, Ring 3 is 5-membered heteroaryl, and Ring 4 is phenyl optionally substituted with one or more R^{4b} or 5- to 6-membered heteroaryl optionally substituted with one or more R^{4b}. In some embodiments, Ring 3 is 5-membered heteroaryl, and Ring 4 is 4- to 6-membered heterocyclyl optionally substituted with one or more R^{4a}, wherein Ring 4 is not tetrahydropyranyl.

In some embodiments of the compound of Formula (I) or a related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), Ring 3 is 5- or 6-membered heterocyclyl optionally substituted with one or more R^{3a} and Ring 4 is phenyl optionally substituted with one or more R^{4b}.

In some embodiments of the compound of Formula (I) or a related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), Ring 3 is 5- or 6-membered heterocyclyl optionally substituted with one or more R^{3a} and Ring 4 is 5- or 6-membered heteroaryl optionally substituted with one or more R^{4b}.

In some embodiments of the compound of Formula (I) or a related formula such as (I-r), (I-s), (I-t), (I-u), (I-v), (I-w), Ring 3 is phenyl optionally substituted with one or more R^{3b}, and Ring 4 is 5- or 6-membered heterocyclyl optionally substituted with one or more R^{4a}.

In some embodiments of the compound of Formula (I) or a related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), Ring 3 is phenyl optionally substituted with one or more R^{3b}, and Ring 4 is 3- to 6-membered cycloalkyl optionally substituted with one or more R^{4a}.

In some embodiments of the compound of Formula (I) or a related formula such as (I-a), (I-b), (I-c), (I-d), (I-e), (If), (I-g), (I-h), (I-j), (I-k), (I-l), (I-m), (I-n), (I-o), (I-p), (I-q), (I-r), (I-s), (I-t), (I-u), (I-v), (I-w), Ring 3 is 5- or 6-membered heteroaryl optionally substituted with one or more R^{3b}, and Ring 4 is phenyl optionally substituted with one or more R^{4b}.

In some embodiments of the compound of Formula (I), or related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), and (I-y), Q is H.

In some embodiments of the compound of Formula (I), or related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), and (I-y), Q is R^{Q}. In some embodiments, R^{Q} is H, halo, OH, C₁₋₄alkyl, or C₁₋₄haloalkyl. In some embodiments, R^{Q} is halo, OH, C₁₋₄alkyl, or C₁₋₄haloalkyl. In some embodiments, R^{Q} is C₁₋₄alkyl or C₁₋₄haloalkyl. In some embodiments, R^{Q} is C₁₋₂alkyl or C₁₋₂haloalkyl. In some embodiments, R^{Q} is methyl. In some embodiments, R^{Q} is H.

In some embodiments of the compound of Formula (I), Q is Ring 5, and the compound is of Formula (II)

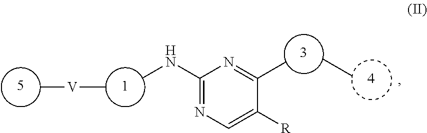

or a tautomer or stereoisomer thereof, or a pharmaceutically acceptable salt of any of the foregoing, wherein Ring 5, V, Ring 1, R, Ring 3, and Ring 4 are as described for Formula (I). In some embodiments, Ring 4 is present.

In some embodiments of the compound of Formula (I), Q is Ring 5, and the compound is of Formula (II)

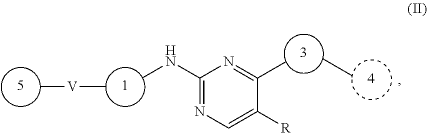

or a tautomer or stereoisomer thereof, or a pharmaceutically acceptable salt of any of the foregoing, wherein:
R is halo;
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Ring 3 is a monocyclic ring selected from the group consisting of 3- to 6-membered cycloalkyl optionally substituted with one or more R^{3a}; 4- to 6-membered heterocyclyl optionally substituted with one or more R^{3a}; phenyl optionally substituted with one or more R^{3b}; or 5- to 6-membered heteroaryl optionally substituted with one or more R^{3b};
Ring 1 is a 4- to 8-membered cycloalkyl or saturated 4- to 8-membered heterocyclyl, each of which is optionally substituted with one or more R¹;
V is a bond, —C₁₋₄alkylene-, —C(O)—, —C(O)O-#, —OC(O)#—C₁₋₄alkylene-C(O)-#, —C(O)N(R^{V})-#, —N(R^{V})C(O)-#, —OC(O)N(R^{V})-#, —N(R^{V})—C(O)O #, —C₁₋₄alkylene-C(O)N(R^{V})-# or —C₁₋₄alkylene-N(R^{V})C(O)-#, wherein # indicates the point of attachment to Ring 1; and
Ring 5 is selected from the group consisting of 3- to 10-membered cycloalkyl optionally substituted with one or more R^{5a}, 4- to 12-membered heterocyclyl optionally substituted with one or more R^{5a}, and 5- to 12-membered heteroaryl optionally substituted with one or more R^{5b};
each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)₁—C₁₋₄alkyl;
each R^{3a}, R^{4a}, and R^{5a} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{3b}, R^{4b}, and R^{5b} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C₁₋₄hydroxyalkyl, C(O)N(R^{y})(R^{z}), CN, N(R^{y})C(O)C₁₋₄alkyl, N(R^{y})C(O)C₁₋₄haloalkyl, S(O)₁R^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R^{V} is independently H, halo, OH, C₁₋₄alkyl, or C₁₋₄haloalkyl;
each R^{w} is independently 3- to 10-membered cycloalkyl, phenyl optionally substituted with halo, or 4- to 12-membered heterocyclyl optionally substituted with hydroxyl, C₁₋₄ hydroxyalkyl, or C₁₋₄aminoalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ-C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, 3- to 10-membered cycloalkyl, or C₁₋₄alkylene-S(O)ₙ—C₁₋₄alkyl; and
each n is independently 0, 1, or 2;
   wherein:
(a) Ring 1 comprises an intraannular amide, or
(b) V comprises an amide or carbamate, or
(c) V is taken together with an atom of Ring 1 or Ring 5 to which it is attached, to form an amide or carbamate;
   and wherein:
when Ring 3 is 5-membered heteroaryl, Ring 4 is present and is not cycloalkyl or tetrahydropyranyl.

In some embodiments of the compound of Formula (I), Q is R^{Q}, and the compound is of Formula (III)

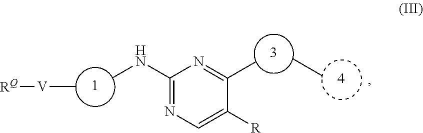

or a tautomer or stereoisomer thereof, or a pharmaceutically acceptable salt of any of the foregoing, wherein:
R is halo;
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Ring 3 is a monocyclic ring selected from the group consisting of 3- to 6-membered cycloalkyl optionally substituted with one or more R^{3a}; 4- to 6-membered heterocyclyl optionally substituted with one or more R^{3a}; phenyl optionally substituted with one or more R^{3b}; or 5- to 6-membered heteroaryl optionally substituted with one or more R^{3b};
Ring 1 is a 4- to 8-membered cycloalkyl or saturated 4- to 8-membered heterocyclyl, each of which is optionally substituted with one or more R¹;
V is a bond, —C₁₋₄alkylene-, —C(O)—, —C(O)O-#, —OC(O)#—C₁₋₄alkylene-C(O)-#, —C(O)N(R^{V})-#, —N(R^{V})C(O)-#, —OC(O)N(R^{V})-#, —N(R^{V})—C(O)O #, —C₁₋₄alkylene-C(O)N(R^{V})-# or —C₁₋₄alkylene-N(R^{V})C(O)-#, wherein # indicates the point of attachment to Ring 1; and
each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{3a}, R^{4a}, and R^{5a} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{3b}, R^{4b}, and R^{5b} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C₁₋₄hydroxyalkyl, C(O)N(R^{y})(R^{z}), CN, N(R^{y})C(O)C₁₋₄alkyl, N(R^{y})C(O)C₁₋₄haloalkyl, S(O)₁R^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R^{Q} is independently H, halo, OH, C₁₋₄alkyl, or C₁₋₄haloalkyl;
each R^{V} is independently H, halo, OH, C₁₋₄alkyl, or C₁₋₄haloalkyl;
each R^{w} is independently 3- to 10-membered cycloalkyl, phenyl optionally substituted with halo, or 4- to 12-membered heterocyclyl optionally substituted with hydroxyl, C₁. 4hydroxyalkyl, or C₁₋₄aminoalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O), —C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, 3- to 10-membered cycloalkyl, or C₁₋₄alkylene-S(O)ₙ-C₁₋₄alkyl; and
each n is independently 0, 1, or 2;
   wherein:
(a) Ring 1 comprises an intraannular amide, or
(b) V comprises an amide or carbamate, or
(c) V is taken together with an atom of Ring 1 to which it is attached, to form an amide or carbamate;
   and wherein:
when Ring 3 is 5-membered heteroaryl, Ring 4 is present and is not cycloalkyl or tetrahydropyranyl;
   and wherein:
when Ring 1 is 8-membered heterocyclyl, Ring 3 is phenyl substituted with CN, Ring 4 is absent, and V is —OC(O)#, then R^{Q} is not tert-butyl.

In some embodiments of the compound of Formula (I), Q is R^{Q}, and the compound is of Formula (III)

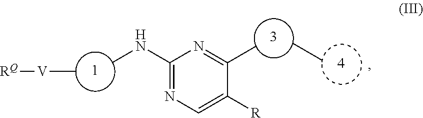

or a tautomer or stereoisomer thereof, or a pharmaceutically acceptable salt of any of the foregoing, wherein:
R is halo;
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Ring 3 is a monocyclic ring selected from the group consisting of 3- to 6-membered cycloalkyl optionally substituted with one or more R^{3a}; 4- to 6-membered heterocyclyl optionally substituted with one or more R^{3a}; phenyl optionally substituted with one or more R^{3b}; or 5- to 6-membered heteroaryl optionally substituted with one or more R^{3b};
Ring 1 is a 4- to 8-membered cycloalkyl or saturated 4- to 8-membered heterocyclyl, each of which is optionally substituted with one or more R¹;
V is a bond, —C₁₋₄alkylene-, —C(O)—, —C(O)O-#, —OC(O)#—C₁₋₄alkylene-C(O)-#, —C(O)N(R^{V})-#, —N(R^{V})C(O)-#, —OC(O)N(R^{V})-#, —N(R^{V})—C(O)O #, —C₁₋₄alkylene-C(O)N(R^{V})-# or —C₁₋₄alkylene-N(R^{V})C(O)-#, wherein # indicates the point of attachment to Ring 1; and
each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{3a}, R^{4a}, and R^{5a} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{3b}, R^{4b}, and R^{5b} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C₁₋₄hydroxyalkyl, C(O)N(R^{y})(R^{z}), CN, N(R^{y})C(O)C₁₋₄alkyl, N(R^{y})C(O)C₁₋₄haloalkyl, S(O)~R^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R^{Q} is independently H, halo, OH, C₁₋₄alkyl, or C₁₋₄haloalkyl;
each R^{V} is independently H, halo, OH, C₁₋₄alkyl, or C₁₋₄haloalkyl;
each R^{w} is independently 3- to 10-membered cycloalkyl, phenyl optionally substituted with halo, or 4- to 12-membered heterocyclyl optionally substituted with hydroxyl, C₁₋₄hydroxyalkyl, or C₁₋₄aminoalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)—C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, 3- to 10-membered cycloalkyl, or C₁₋₄alkylene-S(O)ₙ-C₁₋₄alkyl; and
each n is independently 0, 1, or 2;
   wherein:
(a) Ring 1 comprises an intraannular amide, or
(b) V comprises an amide or carbamate, or
(c) V is taken together with an atom of Ring 1 to which it is attached, to form an amide or carbamate;
   and wherein:
when Ring 3 is 5-membered heteroaryl, Ring 4 is present and is not cycloalkyl or tetrahydropyranyl.

In some embodiments of the compound of Formula (I) or (II), Ring 5 is selected from the group consisting of 3- to 10-membered cycloalkyl optionally substituted with one or more R^{5a}, 4- to 12-membered heterocyclyl optionally substituted with one or more R^{5a}, and 5- to 12-membered heteroaryl optionally substituted with one or more R^{5b}; wherein each R^{5a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo; and each R^{5b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x}.

In some embodiments of the compound of Formula (I) or (II), Ring 5 is C₃₋₁₀ cycloalkyl optionally substituted with one or more R^{5a}, wherein each R^{5a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo. In some embodiments, Ring 5 is C₅₋₁₀ cycloalkyl optionally substituted with one or more R^{5a} wherein each R^{5a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo. In some embodiments, Ring 5 is C₅₋₆ cycloalkyl optionally substituted with one or more R^{5a}, wherein each R^{5a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR¹, or oxo. In some embodiments, Ring 5 is C₆ cycloalkyl optionally substituted with one or more R^{5a} wherein each R^{5a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo.

In some embodiments of the compound of Formula (I) or (II), Ring 5 is 4- to 12-membered heterocyclyl optionally substituted with one or more R^{5a}, wherein each R^{5a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo. In some embodiments, Ring 5 is 5- to 6-membered heterocyclyl optionally substituted with one or more R^{5a}. In some embodiments, Ring 5 is piperidinyl or piperazinyl, each of which is optionally substituted with one or more R^{5a}. In some embodiments, Ring 5 is 10- to 12-membered heterocyclyl optionally substituted with one or more R^{5a}. In some embodiments, Ring 5 is

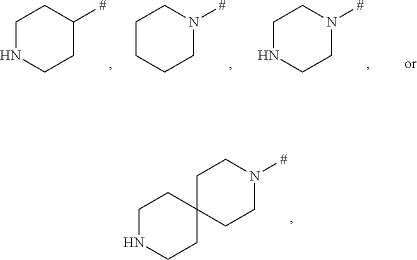

each of which is optionally substituted with one or more R^{5a}, wherein # indicates attachment to V. In some embodiments, Ring 5 is

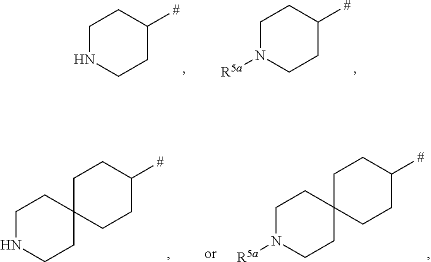

each of which is optionally substituted with one or more R wherein # indicates attachment to V. In some embodiments, Ring 5 is

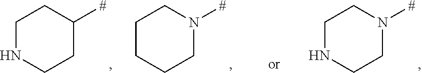

each of which is optionally substituted with one or more R^{5a}, wherein # indicates attachment to V. In some embodiments, Ring 5 is

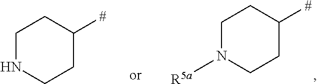

In some embodiments, Ring 5 is

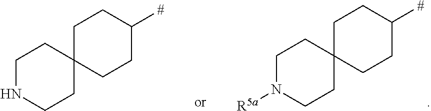

In some embodiments, R^{5a} is C₁₋₄ alkyl or C(O)C₁₋₄alkyl. In some embodiments, R^{5a} is methyl or acetyl.

In some embodiments of the compound of Formula (I) or (II), Ring 5 is 5- to 12-membered heteroaryl optionally substituted with one or more R^{5b}, wherein each R^{5b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x}. In some embodiments, Ring 5 is 5- to 6-membered heteroaryl optionally substituted with one or more R^{5b}.

In some embodiments, Ring 1 is

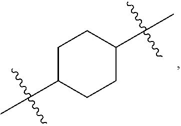 [SMILES: *C1CCC(*)CC1]

and Q is R^{Q}. In some embodiments, Ring 1 is

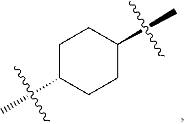

and Q is R^{Q}. In some embodiments, Ring 1 is

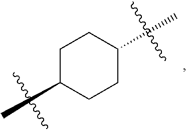

and Q is R^{Q}. In some embodiments, Ring 1 is

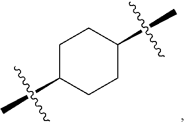

and Q is R^{Q}. In some embodiments, Ring 1 is

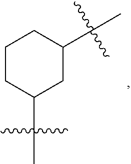 [SMILES: *C1CCCC(*)C1]

and Q is R^{Q}. In some embodiments, Ring 1 is

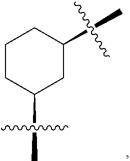

and Q is R^{Q}. In some embodiments, Ring 1 is

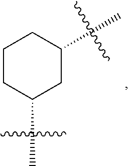

and Q is R^{Q}. In some embodiments, Ring 4 is present.

In some embodiments, Ring 1 is

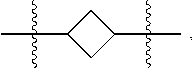 [SMILES: *C1CC(*)C1]

and Q is R^{Q}. In some embodiments, Ring 1 is

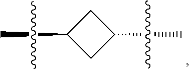

and Q is R^{Q}. In some embodiments, Ring 1 is

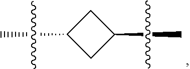

and Q is R^{Q}. In some embodiments, Ring 1 is

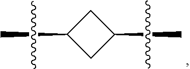

and Q is R^{Q}. In some embodiments, Ring 1 is

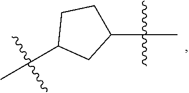 [SMILES: *C1CCC(*)C1]

and Q is R^{Q}. In some embodiments, Ring 1 is

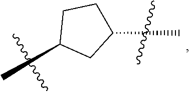

and Q is R^{Q}. In some embodiments, Ring 1 is

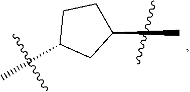

and Q is R^{Q}. In some embodiments, Ring 1 is

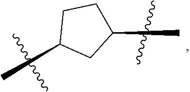

and Q is R^{Q}. In some embodiments, Ring 1 is

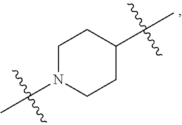 [SMILES: *C1CCN(*)CC1]

and Q is R^{Q}. In some embodiments, Ring 1 is

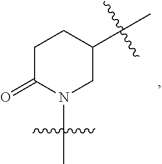 [SMILES: *C1CCC(=O)N(*)C1]

and Q is R^{Q}. In some embodiments, Ring 1 is

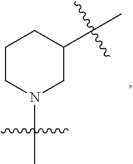 [SMILES: *C1CCCN(*)C1]

and Q is R^{Q}. In some embodiments, Ring 1 is

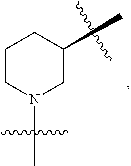 [SMILES: *N1CCC[C@@H](C)C1]

and Q is R^{Q}. In some embodiments, Ring 4 is present.

In some embodiments of the compound of Formula (I) or (II), Ring 1 is a C₄₋₈ cycloalkyl optionally substituted with one or more R¹, Ring 5 is a 4- to 12-membered heterocyclyl optionally substituted with one or more R^{5a}, wherein Ring 5 comprises at least one N atom, and V is —C(O)— and is attached to an annular nitrogen atom of Ring 5, and wherein each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl; and each R^{5a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)~R^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo. In some embodiments, Ring 1 is a C₅₋₈ cycloalkyl optionally substituted with one or more R¹, Ring 5 is a 4- to 12-membered heterocyclyl optionally substituted with one or more R^{5a}, wherein Ring 5 comprises at least one N atom, and V is —C(O)— and is attached to an annular nitrogen atom of Ring 5. In some embodiments, Ring 1 is a C₆₋₈ cycloalkyl optionally substituted with one or more R¹, Ring 5 is a 4- to 12-membered heterocyclyl optionally substituted with one or more R^{5a}, wherein Ring 5 comprises at least one N atom, and V is —C(O)— and is attached to an annular nitrogen atom of Ring 5. In some embodiments, Ring 1 is cyclohexyl or spiro[3.3.]heptanyl, each of which is optionally substituted with one or more R¹, Ring 5 is a 4- to 12-membered heterocyclyl optionally substituted with one or more R^{5a}, wherein Ring 5 comprises at least one N atom, and V is —C(O)— and is attached to an annular nitrogen atom of Ring 5. In some embodiments, Ring 1 is

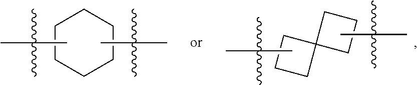

each of which is optionally substituted with one or more R¹, Ring 5 is a 4- to 12-membered heterocyclyl optionally substituted with one or more R^{5a}, wherein Ring 5 comprises at least one N atom, and V is —C(O)— and is attached to an annular nitrogen atom of Ring 5. In some embodiments, Ring 1 is

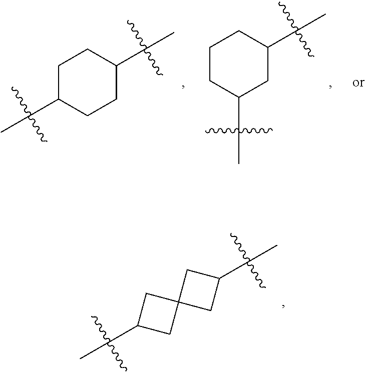 [SMILES: *C1CCC(*)CC1 ; *C1CCCC(*)C1 ; *C1CC2(C1)CC(*)C2]

each of which is optionally substituted with one or more R¹, Ring 5 is a 4- to 12-membered heterocyclyl optionally substituted with one or more R^{5a}, wherein Ring 5 comprises at least one N atom, and V is —C(O)— and is attached to an annular nitrogen atom of Ring 5. In some embodiments, Ring 1 is

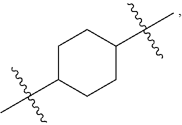 [SMILES: *C1CCC(*)CC1]

Ring 5 is a 4- to 12-membered heterocyclyl optionally substituted with one or more R^{5a}, wherein Ring 5 comprises at least one N atom, and V is —C(O)— and is attached to an annular nitrogen atom of Ring 5. In some embodiments, Ring 1 is

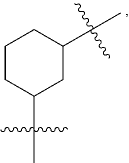 [SMILES: *C1CCCC(*)C1]

Ring 5 is a 4- to 12-membered heterocyclyl optionally substituted with one or more R^{5a}, wherein Ring 5 comprises at least one N atom, and V is —C(O)— and is attached to an annular nitrogen atom of Ring 5. In some embodiments, Ring 1 is

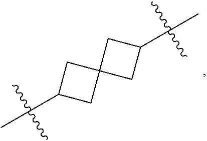 [SMILES: *C1CC2(C1)CC(*)C2]

Ring 5 is a 4- to 12-membered heterocyclyl optionally substituted with one or more R^{5a}, wherein Ring 5 comprises at least one N atom, and V is —C(O)— and is attached to an annular nitrogen atom of Ring 5.

In some embodiments, Q is Ring 5, and Ring 1, V, and Ring 5 are taken together to form

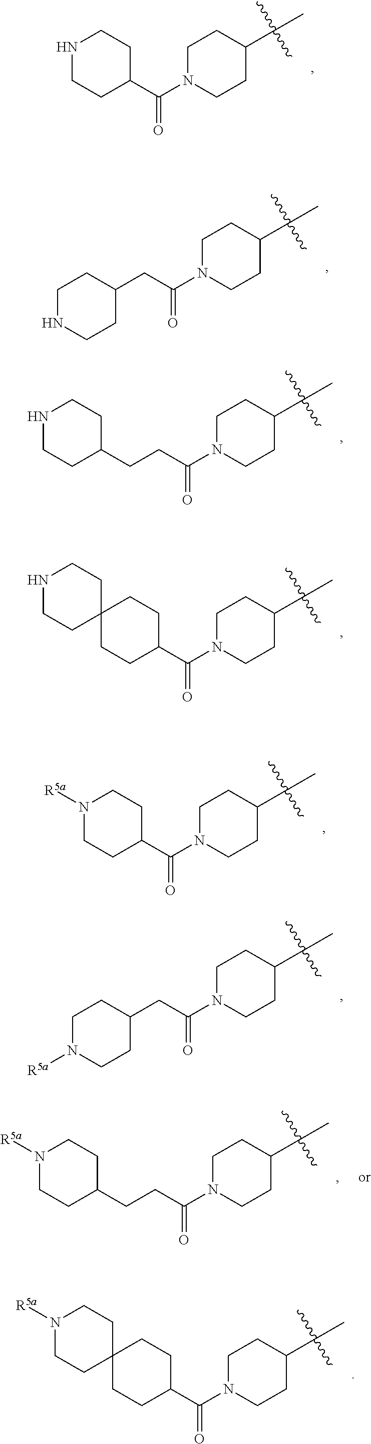

In some embodiments, Ring 4 is present.

In some embodiments of the compound of Formula (I) or (II), Ring 1 is a 4- to 8-membered heterocyclyl ring comprising at least one annular ether and no annular amine or annular amide, optionally substituted with one or more R¹, Ring 5 is a 4- to 12-membered heterocyclyl optionally substituted with one or more R^{5a}, wherein Ring 5 comprises at least one N atom, and V is —C(O)— and is attached to an annular nitrogen atom of Ring 5, and wherein each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)₁—C₁₋₄alkyl; and each R^{5a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo. In some embodiments, Ring 1 is a 5- to 8-membered heterocyclyl ring comprising no annular amine or annular amide, optionally substituted with one or more R¹, Ring 5 is a 4- to 12-membered heterocyclyl optionally substituted with one or more R^{5a}, wherein Ring 5 comprises at least one N atom, and V is —C(O)— and is attached to an annular nitrogen atom of Ring 5. In some embodiments, Ring 1 is a 6- to 8-membered heterocyclyl ring comprising no annular amine or annular amide, optionally substituted with one or more R¹, Ring 5 is a 4- to 12-membered heterocyclyl optionally substituted with one or more R^{5a}, wherein Ring 5 comprises at least one N atom, and V is —C(O)— and is attached to an annular nitrogen atom of Ring 5.

In some embodiments, Ring 1 is unsubstituted with R¹. In some embodiments, Ring 1 is substituted with one or more R¹. In some embodiments, Ring 1 is substituted with 1, 2, or 3 R¹. In some embodiments, each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl. In some embodiments, each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, or N(R^{y})C(O)C₁₋₄haloalkyl. In some embodiments, each R¹ is independently oxo, halo, C₁₋₄alkyl, C₁₋₄haloalkyl, OH, C₁₋₄alkoxy, C₁₋₄haloalkoxy, or CN.

In some embodiments of the compound of Formula (I) or Formula (II), the compound is of Formula (II-a), (II-b), (II-c), (II-d), (II-e), (II-f), or (II-g):

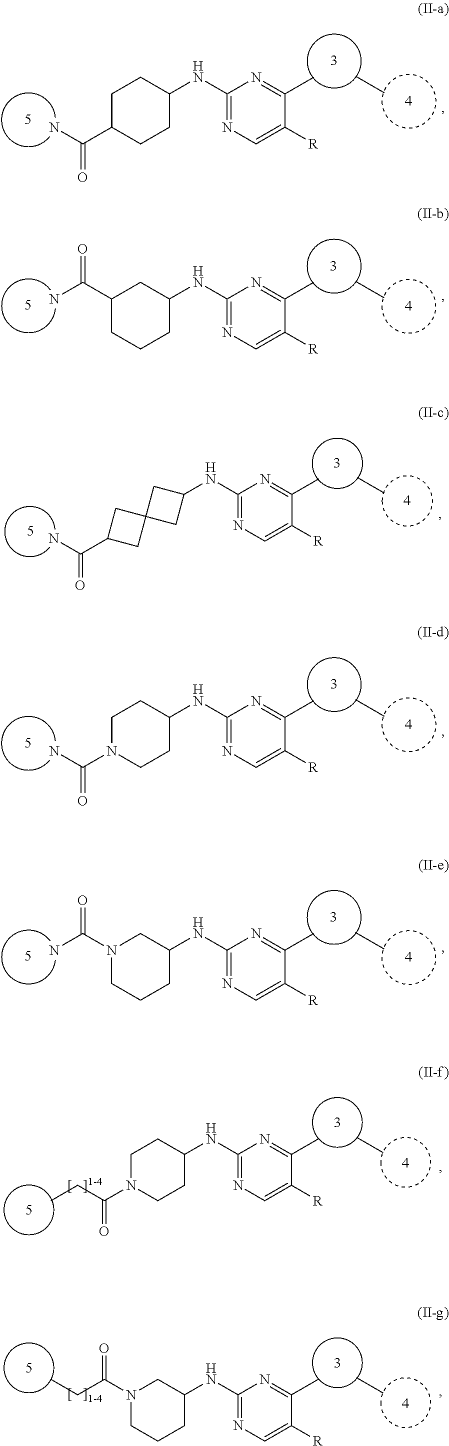

or a stereoisomer thereof, or a pharmaceutically acceptable salt of any of the foregoing, wherein Ring 5 is 4- to 12-membered heterocyclyl optionally substituted with one or more R^{5a}, and R, Ring 3, and Ring 4 are as defined for Formula (I). In some such embodiments, Ring 5 is 5- to 6-membered heterocyclyl optionally substituted with one or more R^{5a}. In some such embodiments, Ring 5 is piperidinyl or piperazinyl, each of which is optionally substituted with one or more R^{5a}. In some embodiments, Ring 4 is present.

In some embodiments of Formula (I) or Formula (II), or any related formula as applicable, when Ring 5 is present, Ring 5, V, and Ring 1 are taken together to form a group of Formula (A′), (B′), (C′), (D′), (E′), (F′), (G′), (H′), or (J′):

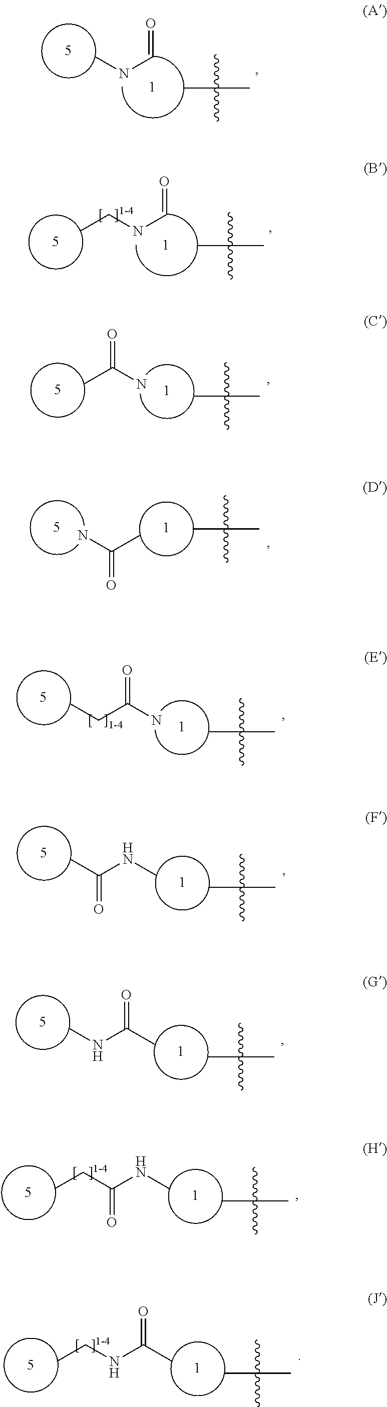

In some embodiments of the compound of Formula (I) or Formula (III), or any related formula as applicable, R^{Q}, V, and Ring 1 are taken together to form a group of Formula (K′), (L′), (N′), or (O′):

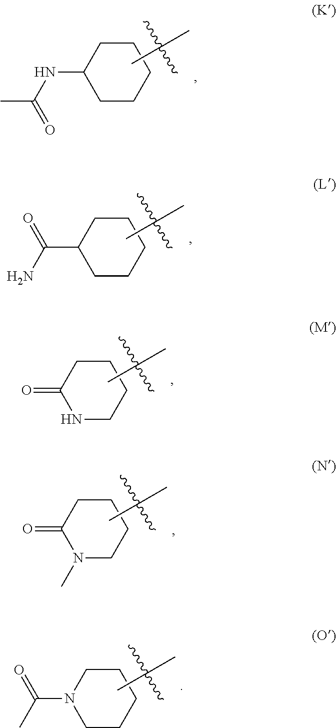

In some embodiments of the compound of Formula (I), Q is of Formula (i),

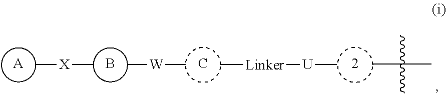

and the compound is of Formula (IV),

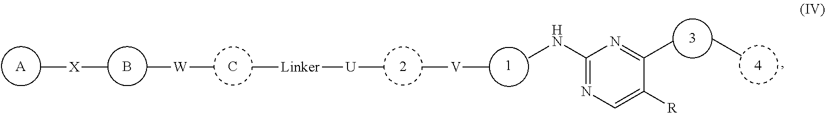

or a tautomer or stereoisomer thereof, or a pharmaceutically acceptable salt of any of the foregoing, wherein Ring A, X, Ring B, W, Ring C, Linker, U, Ring 2, V, Ring 1, R, Ring 3, and Ring 4 are as detailed herein for Formula (I). In some embodiments, Ring 4 is present. In some embodiments, Ring 2 is present. In some embodiments, Ring 2 and Ring 4 are each present. In some embodiments, Ring C is present. In some embodiments, Ring 2, Ring 4, and Ring C are each present.

In some embodiments of the compound of Formula (I), Q is of Formula (i),

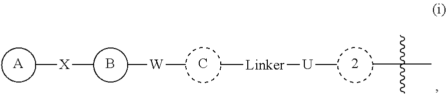

and the compound is of Formula (IV),

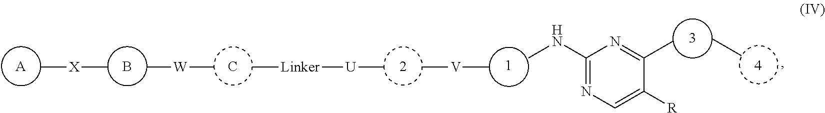

or a tautomer or stereoisomer thereof, or a pharmaceutically acceptable salt of any of the foregoing, wherein:
R is halo;
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Ring 3 is a monocyclic ring selected from the group consisting of 3- to 6-membered cycloalkyl optionally substituted with one or more R^{3a}; 4- to 6-membered heterocyclyl optionally substituted with one or more R^{3a}; phenyl optionally substituted with one or more R^{3b}; or 5- to 6-membered heteroaryl optionally substituted with one or more R^{3b};
Ring 1 is a 4- to 8-membered cycloalkyl or saturated 4- to 8-membered heterocyclyl, each of which is optionally substituted with one or more R¹;
V is a bond, —C₁₋₄alkylene-, —C(O)—, —C(O)O-#, —OC(O)#—C₁₋₄alkylene-C(O)-#, —C(O)N(R^{V})-#, —N(R^{V})C(O)-#, —OC(O)N(R^{V})-#, —N(R^{V})—C(O)O #, —C₁₋₄alkylene-C(O)N(R^{V})-# or —C₁₋₄alkylene-N(R^{V})C(O)-#, wherein # indicates the point of attachment to Ring 1; and
Ring A is selected from the group consisting of

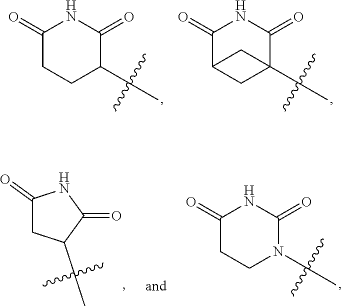 [SMILES: *C1CCC(=O)NC1=O ; *C12CC(C1)C(=O)NC2=O ; *C1CC(=O)NC1=O ; *N1CCC(=O)NC1=O]

each of which is optionally substituted with one or more C₁₋₄alkyl or halo;
X is a bond, —O—, —NH—, or —NHC(O)—;
Ring B is selected from the group consisting of

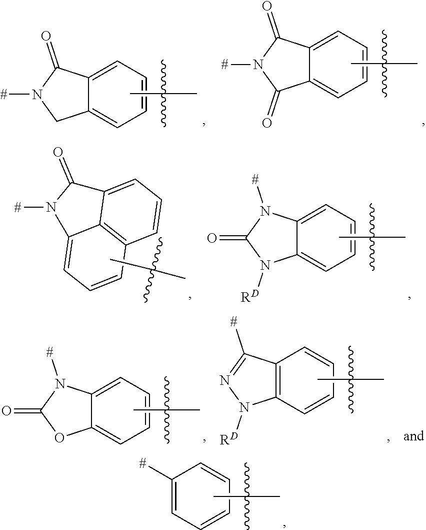

each of which is optionally substituted with one or more halo, C₁₋₄alkyl, C₁₋₄haloalkyl, or OH, and wherein R^{D} is H or C₁₋₄alkyl, wherein # indicates attachment to X;
W is a bond, —O—, —NH—, or —NHC(O)—;
Ring C is optional, and when present is 4- to 12-membered heterocyclyl optionally substituted with one or more R^{C};
   Linker is a bond, —O—, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein
      * indicates the point of attachment to Ring C when Ring C is present, and * indicates the point of attachment to W when Ring C is absent; and
      ** indicates the point of attachment to U when Ring 2 is present, and ** indicates the point of attachment to V when Ring 2 is absent;
      L¹ is a bond or C₁₋₆ alkylene;
      L² is a 3- to 10-membered cycloalkyl or 4- to 12-membered heterocyclyl optionally substituted with one or more R^{L}; and
      L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y});
   U is absent when Ring 2 is absent, and is a bond or C(O) when Ring 2 is present;
   Ring 2 is optional, and when present is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b};
each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ-C₁₋₄alkyl;
each R^{2a}, R^{3a}, and R^{4a} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{2b}, R^{3b}, and R^{4b} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C₁₋₄hydroxyalkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R^{C} is independently halo, OH, C₁₋₄alkyl, or C₁₋₄haloalkyl;
each R^{L} is independently halo, OH, C₁₋₄alkyl, or C₁₋₄haloalkyl;
each R^{V} is independently H, halo, OH, C₁₋₄alkyl, or C₁₋₄haloalkyl;
each R^{w} is independently 3- to 10-membered cycloalkyl, phenyl optionally substituted with halo, or 4- to 12-membered heterocyclyl optionally substituted with hydroxyl, C₁₋₄ hydroxyalkyl, or C₁₋₄aminoalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ-C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, 3- to 10-membered cycloalkyl, or C₁₋₄alkylene-S(O)ₙ-C₁₋₄alkyl; and
each n is independently 0, 1, or 2;
   wherein:
(a) Ring 1 comprises an intraannular amide, or
(b) V comprises an amide or carbamate, or
(c) V is taken together with an atom of Ring 1 or Ring 2 to which it is attached, to form an amide or carbamate;
   and wherein:
when Ring 3 is 5-membered heteroaryl, Ring 4 is present and is not cycloalkyl or tetrahydropyranyl.

In some embodiments of the compound of Formula (I) or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-a), (IV-a), (IV-c), (IV-d), (IV-e), (IV-f), (IV-g), (IV-h), (IV-j), (IV-k), or (IV-l), Ring A is selected from the group consisting of

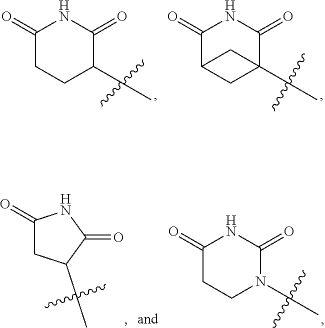 [SMILES: *C1CCC(=O)NC1=O ; *C12CC(C1)C(=O)NC2=O ; *C1CC(=O)NC1=O ; *N1CCC(=O)NC1=O]

each of which is optionally substituted with C₁₋₄alkyl or halo. In some embodiments, Ring A is

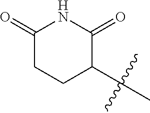 [SMILES: *C1CCC(=O)NC1=O]

optionally substituted with C₁₋₄alkyl or halo. In some embodiments, Ring A is

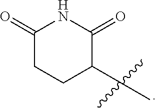 [SMILES: *C1CCC(=O)NC1=O]

In some embodiments of the compound of Formula (I) or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-a), (IV-a), (IV-c), (IV-d), (IV-e), (IV-f), (IV-g), (IV-h), (IV-j), (IV-k), or (IV-l), X is a bond, —O—, —NH—, or —NHC(O)—. In some embodiments, X is a bond or —NH—.

In some embodiments of the compound of Formula (I) or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-a), (IV-a), (IV-c), (IV-d), (IV-e), (IV-f), (IV-g), (IV-h), (IV-j), (IV-k), or (IV-l), Ring B is selected from the group consisting of

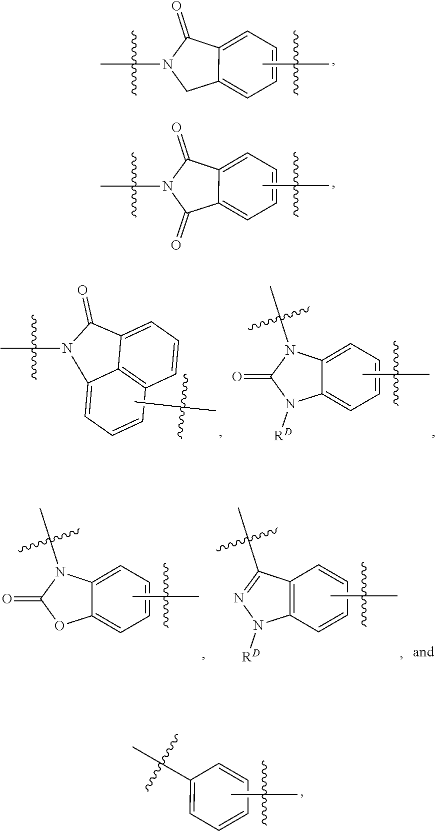

each of which is optionally substituted with one or more halo or OH, wherein R^{D} is H or C₁₋₄alkyl. In some embodiments, Ring B is selected from the group consisting of

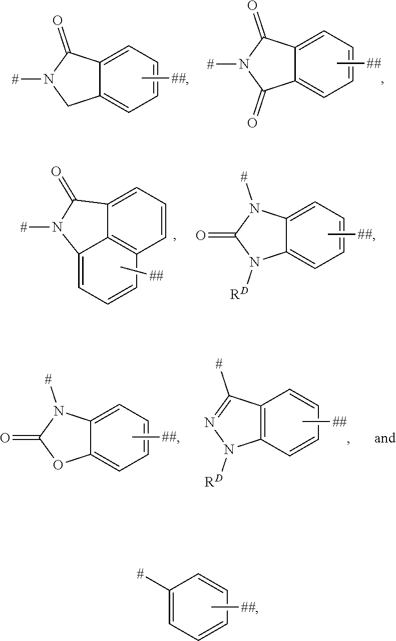

each of which is optionally substituted with one or more halo or OH, wherein R^{D} is H or C₁₋₄alkyl, # indicates attachment to X, and ## indicates attachment to W. In some embodiments, Ring B is

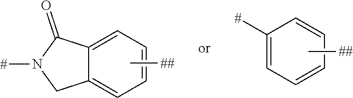

optionally substituted with one or more halo or OH. In some embodiments, Ring B is

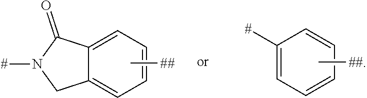

In some embodiments of the compound of Formula (I) or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-a), (IV-a), (IV-c), (IV-d), (IV-e), (IV-f), (IV-g), (IV-h), (IV-j), (IV-k), or (IV-l), W is a bond, —O—, —NH—, or —NHC(O)—. In some embodiments, W is a bond or —O—.

In some embodiments of the compound of Formula (I) or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-a), (IV-d), (IV-e), (IV-f), (IV-g), (IV-h), (IV-j), (IV-k), or (IV-l), Ring C is absent.

In some embodiments of the compound of Formula (I) or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-a), (IV-c), (IV-e), (IV-f), (IV-g), (IV-h), (IV-j), (IV-k), or (IV-l), Ring C is present and is 4- to 12-membered heterocyclyl optionally substituted with one or more R^{C}, wherein each R^{C} is independently halo, OH, C₁₋₄alkyl, or C₁₋₄haloalkyl. In some embodiments, Ring C is 4- to 12-membered nitrogen-containing heterocyclyl optionally substituted with one or more R^{C}. In some embodiments, Ring C is 5- to 6-membered heterocyclyl optionally substituted with one or more R^{C}. In some embodiments, Ring C is

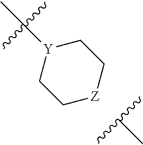

optionally substituted with one or more R^{C}, wherein Y and Z are independently CH or N. In some embodiments, Ring C is piperidinyl or piperazinyl, each of which is optionally substituted with one or more R^{C}. In some embodiments, Ring C is piperidinyl optionally substituted with one or more R^{C}. In some embodiments, Ring C is piperazinyl optionally substituted with one or more R^{C}. In some embodiments, Ring C is

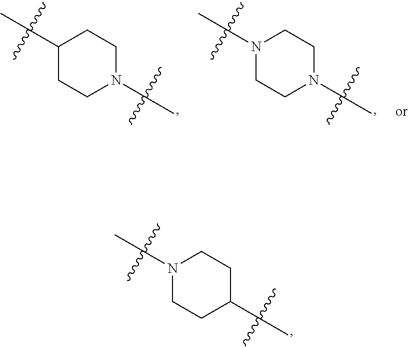 [SMILES: *C1CCN(*)CC1 ; *N1CCN(*)CC1 ; *C1CCN(*)CC1]

each of which is optionally substituted with one or more R^{C}. In some embodiments, Ring C is

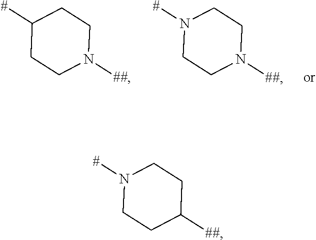

each of which is optionally substituted with one or more R^{C}, wherein # indicates attachment to W and ## indicates attachment to Linker. In some embodiments, Ring C is

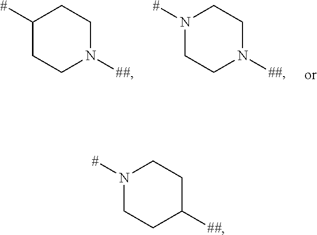

wherein # indicates attachment to W and ## indicates attachment to Linker.

In some embodiments of the compound of Formula (I) or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-a), (IV-a), (IV-c), (IV-d), (IV-e), (IV-f), (IV-g), (IV-h), (IV-j), (IV-k), or (IV-l), Linker is a bond, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein * indicates attachment to Ring C when Ring C is present, or attachment to W when Ring C is absent; and ** indicates attachment to U when Ring 2 is present, or attachment to V when Ring 2 is absent; L¹ is a bond or C₁₋₆ alkylene; L² is a 3- to 10-membered cycloalkyl or 4- to 12-membered heterocyclyl optionally substituted with one or more R^{L}, wherein each R^{L} is independently halo, OH, C₁₋₄alkyl, or C₁₋₄haloalkyl; and L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y}).

In some embodiments of the compound of Formula (I′), (I), or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (IV-a), (IV-a), (IV-c), (IV-d), (IV-e), (IV-f), (IV-g), (IV-h), (IV-j), (IV-k), or (IV-l), Linker is a bond, —O—, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein * indicates attachment to Ring C when Ring C is present, or attachment to W when Ring C is absent; and ** indicates attachment to U when Ring 2 is present, or attachment to V when Ring 2 is absent; L¹ is a bond or C₁₋₆ alkylene; L² is a 3- to 10-membered cycloalkyl or 4- to 12-membered heterocyclyl optionally substituted with one or more R^{L}, wherein each R^{L} is independently halo, OH, C₁₋₄alkyl, or C₁₋₄haloalkyl; and L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y}).

In some embodiments of the compound of Formula (I) or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-a), (IV-a), (IV-c), (IV-d), (IV-e), (IV-f), (IV-g), (IV-h), (IV-j), (IV-k), or (IV-l), Linker is a bond, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}). In some embodiments of the compound of Formula (I) or (IV), Linker is a bond, C₁₋₁₀alkylene, or C(O)C₁₋₁₀alkylene.

In some embodiments of the compound of Formula (I) or (IV), Linker is a bond, C₁₋₁₀alkylene, C(O)—C₁₋₁₀alkylene, C₁₋₆alkylene-C(O)—C₁₋₁₀alkylene, C₀₋₆alkylene-C(O)N(R^{y})—C₁₋₁₀alkylene, or is of the formula *-L¹-L²-L³-**, wherein L¹ is a bond or C₁₋₆ alkylene, L² is a piperidinyl ring or piperazinyl ring, and L³ is C₁₋₁₀ alkylene. In some embodiments, Linker is a bond, C₁₋₂alkylene, C(O)—C₁₋₂alkylene, or C₁₋₂alkylene-C(O)N(R^{y})—C₁₋₁₀alkylene.

In some embodiments of the compound of Formula (I) or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-c), or (IV-d), Ring 2 is absent.

In some embodiments of the compound of Formula (I) or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-a), (IV-a), (IV-e), (IV-f), (IV-g), (IV-h), (IV-j), (IV-k), or (IV-l), Ring 2 is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b}, wherein each R^{2a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo; and each R^{2b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x}; and U is a bond. In some embodiments, the 4- to 12-membered heterocyclyl optionally substituted with one or more R^{2a} is a nitrogen-containing heterocyclyl.

In some embodiments of the compound of Formula (I) or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-a), (IV-a), (IV-e), (IV-f), (IV-g), (IV-h), (IV-j), (IV-k), or (IV-l), Ring 2 is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b}, wherein each R^{2a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo; and each R^{2b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)~R^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x}; and U is C(O). In some embodiments, the 4- to 12-membered heterocyclyl optionally substituted with one or more R^{2a} is a nitrogen-containing heterocyclyl.

In some embodiments of the compound of Formula (I) or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-a), (IV-a), (IV-e), (IV-f), (IV-g), (IV-h), (IV-j), (IV-k), or (IV-l), Ring 2 is C₃₋₁₀ cycloalkyl optionally substituted with one or more R^{5a}, wherein each R^{2a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo. In some embodiments, Ring 2 is C₅₋₁₀ cycloalkyl optionally substituted with one or more R^{5a}. In some embodiments, Ring 2 is C₅₋₆ cycloalkyl optionally substituted with one or more R^{5a}. In some embodiments, Ring 2 is C₆ cycloalkyl optionally substituted with one or more R^{5a}.

In some embodiments, Ring 2 is

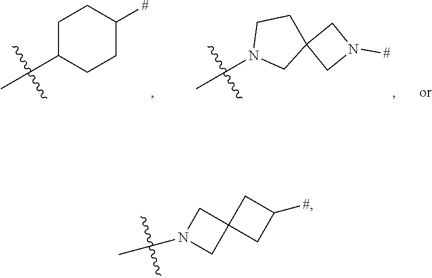

each of which is optionally substituted with one or more R^{2a}, wherein # indicates attachment to V.

In some embodiments of the compound of Formula (I) or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-a), (IV-a), (IV-e), (IV-f), (IV-g), (IV-h), (IV-j), (IV-k), or (IV-l), Ring 2 is 4- to 12-membered heterocyclyl optionally substituted with one or more R^{2a}, wherein each R^{2a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo. In some embodiments, Ring 2 is 5- to 6-membered heterocyclyl optionally substituted with one or more R^{2a}. In some embodiments, Ring 2 is 4- to 12-membered nitrogen-containing heterocyclyl optionally substituted with one or more R^{2a}. In some embodiments, Ring 2 is 5- to 6-membered nitrogen-containing heterocyclyl optionally substituted with one or more R^{2a}. In some embodiments, Ring 2 is piperidinyl or piperazinyl, each of which is optionally substituted with one or more R^{2a}. In some embodiments, Ring 2 is 10- to 12-membered heterocyclyl optionally substituted with one or more R^{2a}. In some embodiments, Ring 2 is

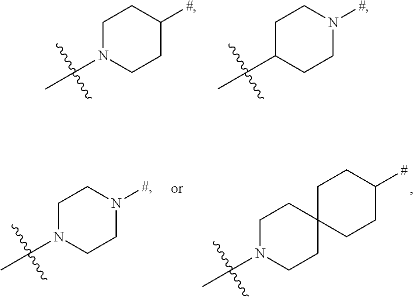

each of which is optionally substituted with one or more R^{2a}, wherein # indicates attachment to V. In some embodiments, Ring 2 is

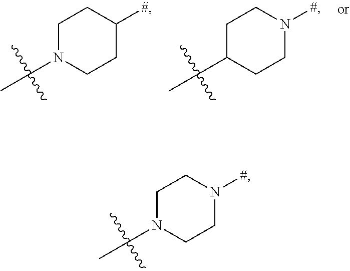

each of which is optionally substituted with one or more R^{2a}, wherein # indicates attachment to V. In some embodiments, Ring 2 is

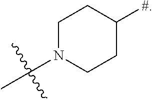

In some embodiments, Ring 2 is

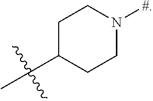

In some embodiments, Ring 2 is

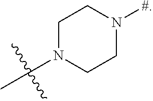

In some embodiments, Ring 2 is

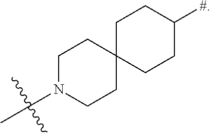

In some embodiments of the compound of Formula (I) or (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-a), (IV-a), (IV-e), (IV-f), (IV-g), (IV-h), (IV-j), (IV-k), or (IV-l), Ring 2 is 5- to 12-membered heteroaryl optionally substituted with one or more R^{5b}, wherein each R^{2b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)~R^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x}. In some embodiments, Ring 2 is 5- to 6-membered heteroaryl optionally substituted with one or more R^{5b}.

In some embodiments of the compound of Formula (I′), or any related formula such as (I), (IV), (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (IV-a), (IV-a), (IV-e), (IV-f), (IV-g), (IV-h), (IV-j), (IV-k), or (IV-l), each R^{2a}, R^{3a}, R^{4a}, and R^{5a} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C(O)N(R^{y})(R^{z}), CN, N(R^{y})C(O)C₁₋₄alkyl, N(R^{y})C(O)C₁₋₄haloalkyl, S(O)ₙR^{x}, S(O)ₐN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo. In some embodiments, each R^{2a}, R^{3a}, R^{4a}, and R^{5a} is C(O)R^{w}. In some embodiments, each R^{3a} is C(O)R^{w}.

In some embodiments of the compound of Formula (I′), or any related formula such as (I), (IV), (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (IV-a), (IV-a), (IV-e), (IV-f), (IV-g), (IV-h), (IV-j), (IV-k), or (IV-l), each R^{2b}, R^{3b}, R^{4b}, and R^{5b} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C₁₋₄hydroxyalkyl, C(O)N(R^{y})(R^{z}), CN, N(R^{y})C(O)C₁₋₄alkyl, N(R^{y})C(O)C₁₋₄haloalkyl, S(O)~R^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x}. In some embodiments, each R^{2b}, R^{3b}, R^{4b}, and R^{5b} is independently C(O)R^{w} or C₁₋₄hydroxyalkyl. In some embodiments, each R^{2b}, R^{3b}, R^{4b}, and R^{5b} is independently C(O)R^{w}. In some embodiments, each R^{2b}, R^{3b}, R^{4b}, and R^{5b} is independently C₁₋₄hydroxyalkyl. In some embodiments, each R^{3b} is independently C(O)R^{w} or C₁₋₄hydroxyalkyl. In some embodiments, each R^{3b} is independently C(O)R^{w}. In some embodiments, each R^{3b} is independently C₁₋₄hydroxyalkyl. In some embodiments, each R^{3b} is

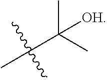 [SMILES: *C(C)(C)O]

In some embodiments, each R^{w} is independently 3- to 10-membered cycloalkyl, phenyl optionally substituted with halo, or 4- to 12-membered heterocyclyl optionally substituted with hydroxyl, C₁₋₄hydroxyalkyl, or C₁₋₄aminoalkyl. In some embodiments, each R^{w} is independently 3- to 10-membered cycloalkyl. In some embodiments, each R^{w} is independently phenyl optionally substituted with halo. In some embodiments, each R^{w} is phenyl optionally substituted with fluoro or chloro. In some embodiments, each R^{w} is phenyl optionally substituted with fluoro. In some embodiments, each R^{w} is phenyl optionally substituted with chloro. In some embodiments, each R^{w} is independently 4- to 12-membered heterocyclyl optionally substituted with hydroxyl, C₁₋₄hydroxyalkyl, or C₁₋₄aminoalkyl. In some embodiments, each R^{w} is 4- to 12-membered heterocyclyl optionally substituted with hydroxyl. In some embodiments, each R^{w} is 4- to 12-membered heterocyclyl optionally substituted with C₁₋₄hydroxyalkyl. In some embodiments, each R^{w} is 4- to 12-membered heterocyclyl optionally substituted with C₁₋₄aminoalkyl. In some embodiments, R^{w} is

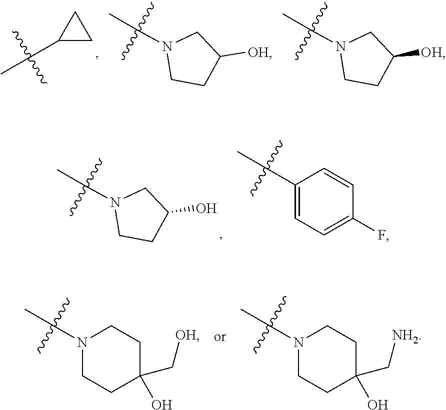

In some embodiments, each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, 3- to 10-membered cycloalkyl, or C₁₋₄alkylene-S(O)ₙ-C₁₋₄alkyl. In some embodiments, each R^{y} and R^{z} is independently 3- to 10-membered cycloalkyl. In some embodiments, one of R^{y} and R^{z} is hydrogen and the other is 3- to 10-membered cycloalkyl. In some embodiments, one of R^{y} and R^{z} is hydrogen and the other is cyclopropyl.

In some embodiments, each R^{V} is independently H, halo, OH, C₁₋₄alkyl, or C₁₋₄haloalkyl. In some embodiments, each R^{V} is independently H or C₁₋₄alkyl. In some embodiments, each R^{V} is independently H or C₁₋₂alkyl. In some embodiments, each R^{V} is independently H or methyl. In some embodiments, each R^{V} is independently H or ethyl. In some embodiments, each R^{V} is H. In some embodiments, each R^{V} is methyl. In some embodiments, each R^{V} is ethyl.

In some embodiments of Formula (IV), Ring C, U, and Ring 2 are present, and the compound is of Formula (IV-a). In some embodiments of Formula (IV), U and Ring 2 are present, and Ring C is absent, and the compound is of Formula (IV-a). In some embodiments, Ring C is present, and U and Ring 2 are absent, and the compound is of Formula (IV-c). In some embodiments, Ring C, U, and Ring 2 are absent, and the compound is of Formula (IV-d). In some embodiments, the compound of Formula (I) or (IV) is of Formula (IV-a), (IV-a), (IV-c), or (IV-d):

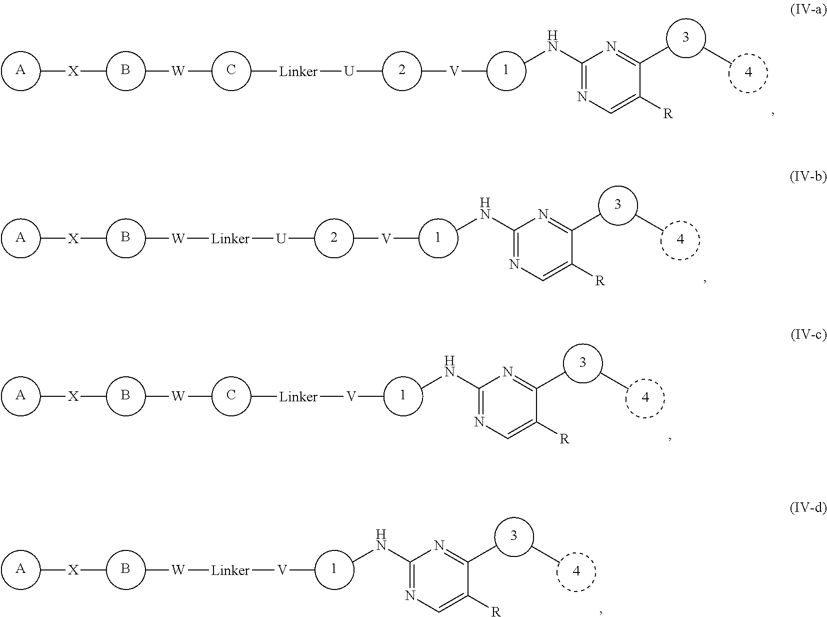

wherein Ring A, X, Ring B, W, Ring C, Linker, U, Ring 2, V, Ring 1, R, Ring 3, and Ring 4 are as described herein for Formula (I).

In some embodiments of Formula (I), Formula (IV), or any related formula such as Formula (IV-a), (IV-a), (IV-e), (IV-f), (IV-g), (IV-h), (IV-j), (IV-k), or (IV-l), when Ring 2 is present, Ring 2, V, and Ring 1 are taken together to form a group of Formula (A), (B), (C), (D), (E), (F), (G), (H), or (J):

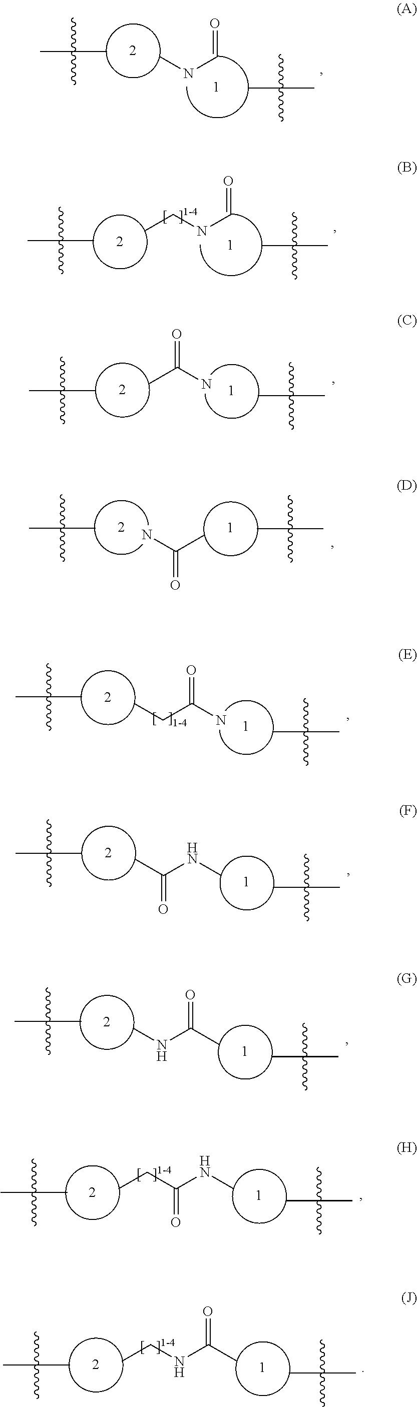

In some such embodiments, Ring 2, V, and Ring 1 are taken together to form a group of Formula (C), (D), or (E). In some such embodiments, when Ring 2 is present, Ring 2, V, and Ring 1 are taken together to form a group of Formula (K), (L), (M), or (N):

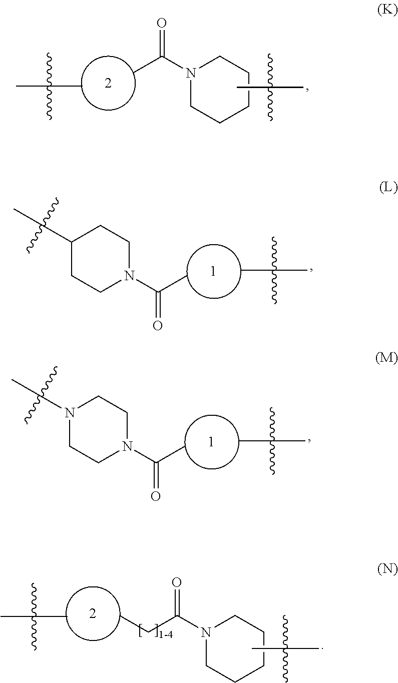

In some such embodiments, when Ring 2 is present, Ring 2, V, and Ring 1 are taken together to form a group of Formula (O), (P), (Q), or (R), or a stereoisomer thereof:

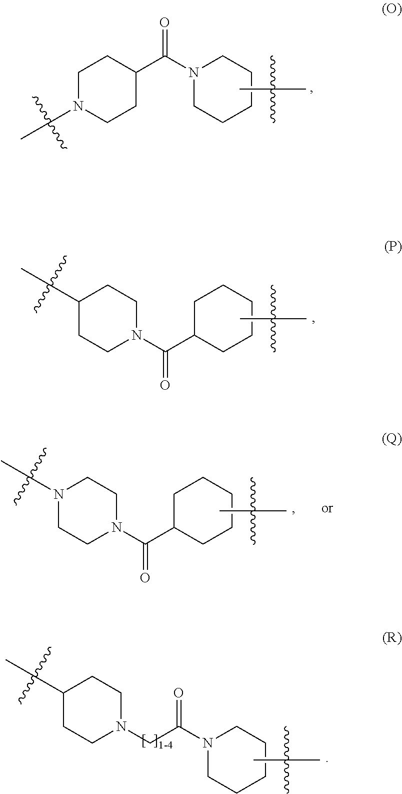

In some embodiments of the compound of Formula (I), (II), (III), (IV), or any related formula such as (IV-a), (IV-a), (IV-c), (IV-d), (IV-e), (IV-f), (IV-g), (IV-h), (IV-j), (IV-k), or (IV-l), Ring 1 is a saturated 4- to 8-membered heterocyclyl comprising an annular amide, optionally substituted with one or more R¹, and V is a bond or C₁₋₄ alkylene. In some embodiments, Ring 1 is a saturated 5- to 6-membered heterocyclyl comprising an annular amide, optionally substituted with one or more R¹, and V is a bond or C₁₋₄ alkylene. In some embodiments, Ring 1 is

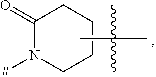

optionally substituted with one or more R¹, wherein # indicates attachment to V, which is a bond or C₁₋₄ alkylene. In some embodiments, Ring 1 is

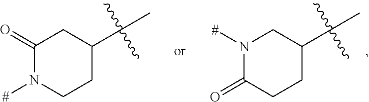

each of which is optionally substituted with one or more R¹, and V is a bond or C₁₋₄ alkylene. In some embodiments, Ring 1 is

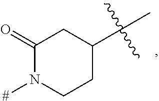

and V is a bond or C₁₋₄ alkylene. In some embodiments, Ring 1 is

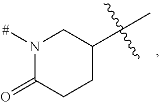

and V is a bond or C₁₋₄ alkylene.

In some embodiments of the compound of Formula (I), (II), (III), (IV), or any related formula such as (IV-a), (IV-a), (IV-c), or (IV-d), Ring 1 is a saturated 4- to 8-membered heterocyclyl comprising at least one annular amine, optionally substituted with one or more R¹, and V is —C(O)— or —C₁₋₄alkylene-C(O)—. In some embodiments of the compound of Formula (I), Ring 1 is a saturated 5- to 8-membered heterocyclyl comprising at least one annular amine, optionally substituted with one or more R¹, and V is —C(O)— or —C₁₋₄alkylene-C(O)—. In some embodiments of the compound of Formula (I), Ring 1 is a saturated 6- to 8-membered heterocyclyl comprising at least one annular amine, optionally substituted with one or more R¹, and V is —C(O)— or —C₁₋₄alkylene-C(O)—. In some embodiments, Ring 1 is piperidinyl or 2-azaspiro[3.3]heptanyl, each of which is optionally substituted with one or more R¹, and V is —C(O)— or —C₁₋₄alkylene-C(O)—. In some embodiments, Ring 1 is

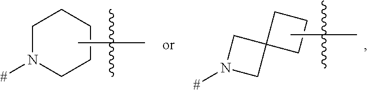

wherein # indicates attachment to V, each of which is optionally substituted with one or more R¹, and V is —C(O)— or —C₁₋₄alkylene-C(O)—. In some embodiments, Ring 1 is

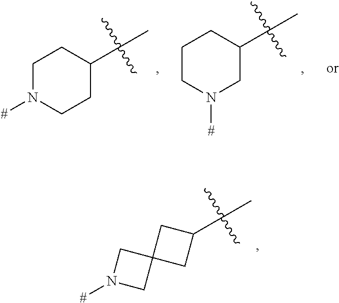

wherein # indicates attachment to V, each of which is optionally substituted with one or more R¹, and V is —C(O)— or —C₁₋₄alkylene-C(O)—. In some embodiments, Ring 1 is

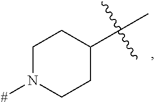

and V is —C(O)— or —C₁₋₄alkylene-C(O)—. In some embodiments, Ring 1 is

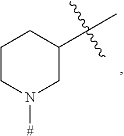

and V is —C(O)— or —C₁₋₄alkylene-C(O)—. In some embodiments, Ring 1 is

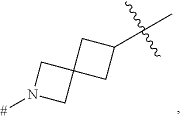

### and V is —C(O)— or —C₁₋₄alkylene-C(O)—.

In some embodiments of the compound of Formula (I), (II), (III), (IV), or any related formula such as (IV-a), (IV-a), (IV-c), or (IV-d), Ring 1 is a C₄₋₈ cycloalkyl optionally substituted with one or more R¹, and V is —C(O)N(R^{V})-#, —N(R^{V})C(O)-#, —C₁₋₄alkylene-C(O)N(R^{V})-# or —C₁₋₄alkylene-N(R^{V})C(O)-#.

In some embodiments of the compound of Formula (I), (IV), or any related formula such as (IV-a) or (IV-a), Ring 1 is a C₄₋₈ cycloalkyl optionally substituted with one or more R¹, Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is a C₅₋₈ cycloalkyl optionally substituted with one or more R¹, Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is a C₆₋₈ cycloalkyl optionally substituted with one or more R¹, Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is cyclohexyl or spiro[3.3.]heptanyl, each of which is optionally substituted with one or more R¹, Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is

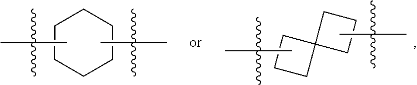

each of which is optionally substituted with one or more R¹, Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is

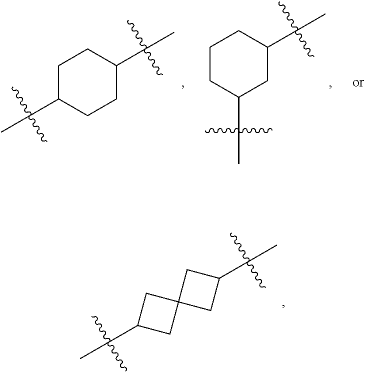 [SMILES: *C1CCC(*)CC1 ; *C1CCCC(*)C1 ; *C1CC2(C1)CC(*)C2]

each of which is optionally substituted with one or more R¹, Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is

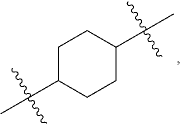 [SMILES: *C1CCC(*)CC1]

Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is

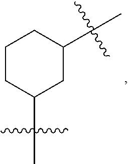 [SMILES: *C1CCCC(*)C1]

Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is

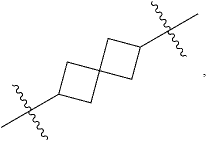 [SMILES: *C1CC2(C1)CC(*)C2]

Ring 2 is a 4- to 12-membered nitrogen-containing heterocyclyl optionally substituted with one or more R^{2a}, and V is —C(O)— and is attached to an annular nitrogen atom of Ring 2.

In some embodiments of the compound of Formula (I), (IV), or any related formula such as (IV-a) or (IV-a), Ring 1 is a C₄₋₈ cycloalkyl optionally substituted with one or more R¹, Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is a C₅₋₈ cycloalkyl optionally substituted with one or more R¹, Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is a C₆₋₈ cycloalkyl optionally substituted with one or more R¹, Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is cyclohexyl or spiro[3.3.]heptanyl, each of which is optionally substituted with one or more R¹, Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is

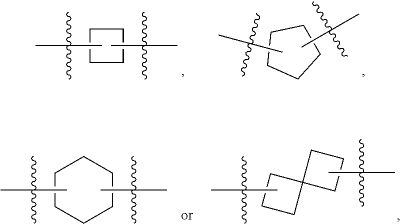

each of which is optionally substituted with one or more R¹, Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is

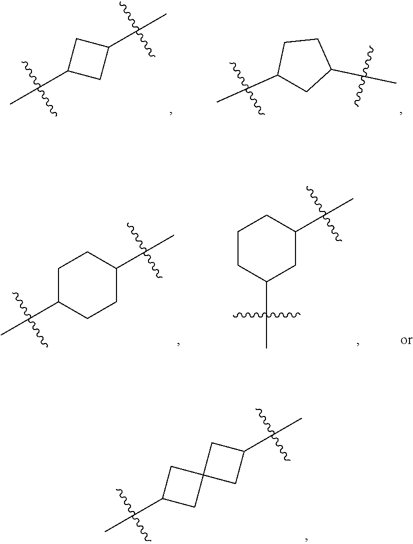 [SMILES: *C1CC(*)C1 ; *C1CCC(*)C1 ; *C1CCC(*)CC1 ; *C1CCCC(*)C1 ; *C1CC2(C1)CC(*)C2]

each of which is optionally substituted with one or more R¹, Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is

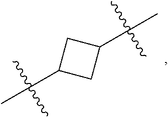 [SMILES: *C1CC(*)C1]

Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is

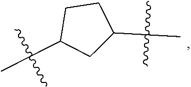 [SMILES: *C1CCC(*)C1]

Ring 2 is a 4- to 12-membered nitrogen-containing heterocyclyl optionally substituted with one or more R^{2a}, and V is —C(O)— and is attached to an annular nitrogen atom of Ring 2. In some embodiments, Ring 1 is a C₄₋₈ cycloalkyl optionally substituted with one or more R¹, Ring 2 is a C₄₋₈ cycloalkyl optionally substituted with one or more R^{2a}, and V is —C(O)N(R^{V})-#, wherein # indicates the point of attachment to Ring 1. In some embodiments, Ring 1 is a C₅₋₈ cycloalkyl optionally substituted with one or more R¹, Ring 2 is a C₅₋₈ cycloalkyl optionally substituted with one or more R^{a} and V is —C(O)N(R^{V})-#, wherein # indicates the point of attachment to Ring 1. In some embodiments, Ring 1 is a C₆₋₈ cycloalkyl optionally substituted with one or more R¹, Ring 2 is a C₆₋₈ cycloalkyl optionally substituted with one or more R^{2a}, and V is —C(O)N(R^{V})-#, wherein # indicates the point of attachment to Ring 1. In some embodiments, Ring 1 is a cyclohexyl optionally substituted with one or more R¹, Ring 2 is a cyclohexyl optionally substituted with one or more R^{2a}, and V is —C(O)N(R^{V})-#, wherein # indicates the point of attachment to Ring 1.

In some embodiments of the compound of Formula (I) or Formula (IV), Ring 2 is present and the compound is of Formula (IV-e), (IV-f), (IV-g), (IV-h), (IV-j), (IV-k), or (IV-l):

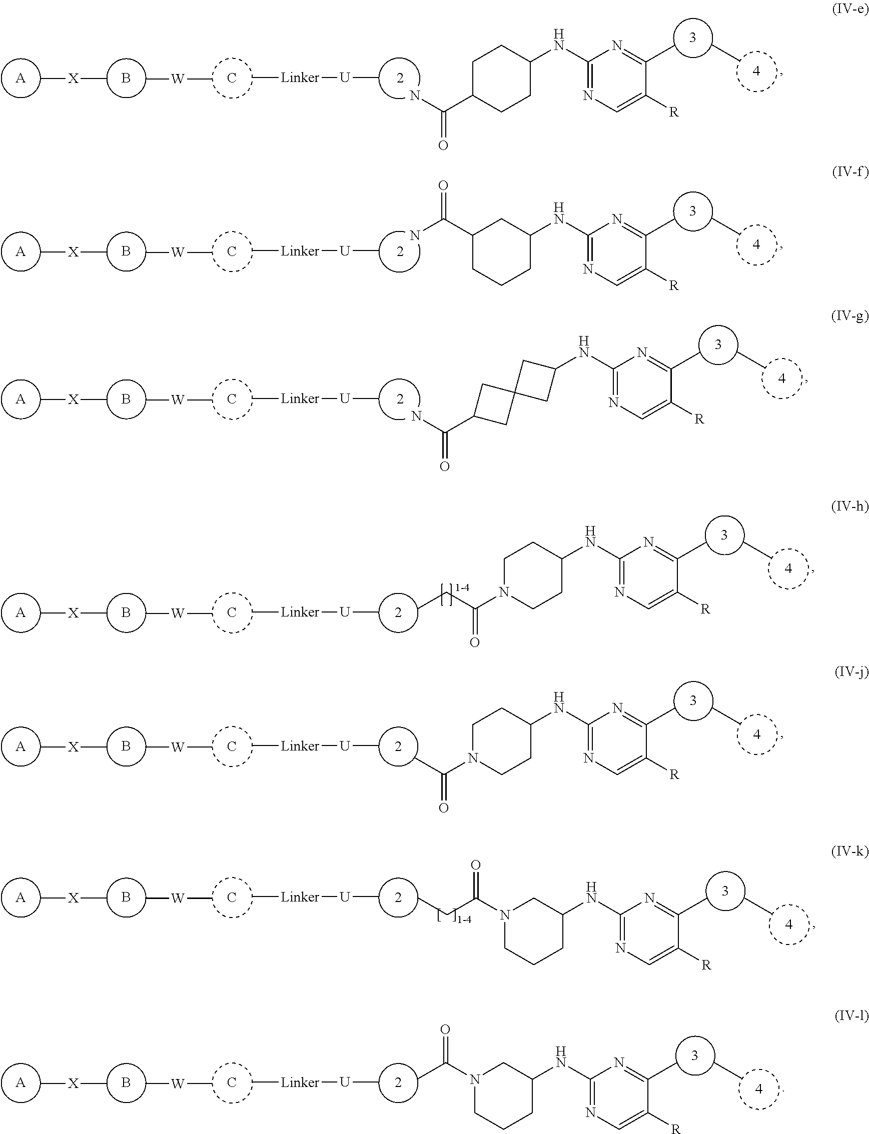

In some embodiments of the compound of Formula (I), (II), (III), (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), Ring 3 is 3- to 6-membered cycloalkyl optionally substituted with one or more R^{3a}, 4- to 6-membered heterocyclyl optionally substituted with one or more R^{3a}, phenyl optionally substituted with one or more R^{3b}, or 5- to 6-membered heteroaryl optionally substituted with one or more R^{3b}, wherein each R^{3a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₐN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo, and each R^{3b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)~R^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x}. In some embodiments, each R^{3a} is independently halo, methyl, trifluoromethyl, OH, OCH₃, acetyl, CN, or oxo. In some embodiments, each R^{3a} is oxo. In some embodiments, each R^{3b} is independently halo, methyl, trifluoromethyl, OH, OCH₃, acetyl, or CN.

In some embodiments of the compound of Formula (I), (II), (III), (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), Ring 3 is 3- to 6-membered cycloalkyl optionally substituted with one or more R^{3a}.

In some embodiments of the compound of Formula (I), (II), (III), (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), Ring 3 is 4- to 6-membered heterocyclyl optionally substituted with one or more R^{3a}. In some embodiments of the compound of Formula (I), Ring 3 is 4- to 6-membered nitrogen-containing heterocyclyl optionally substituted with one or more R^{3a}. In some embodiments, Ring 3 is pyrrolidinyl, piperidinyl, piperazinyl, or morpholinyl. In some embodiments, Ring 3 is

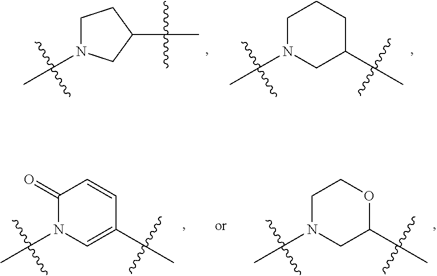 [SMILES: *C1CCN(*)C1 ; *C1CCCN(*)C1 ; *c1ccc(=O)n(*)c1 ; *C1CN(*)CCO1]

each of which is optionally substituted with one or more R^{3a}. In some embodiments, Ring 3 is

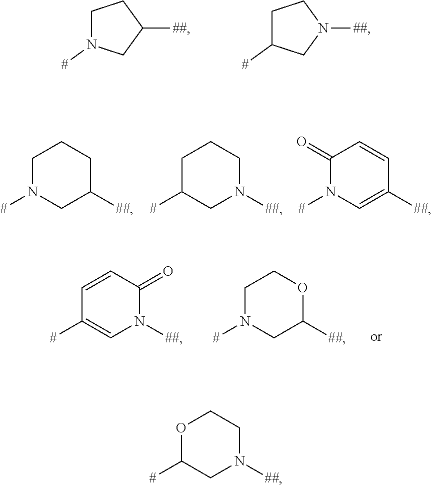

each of which is optionally substituted with one or more R^{3a}, wherein # indicates attachment to the pyrimidine ring of Formula (I), and ## indicates attachment to Ring 4, when present. In some embodiments, Ring 3 is

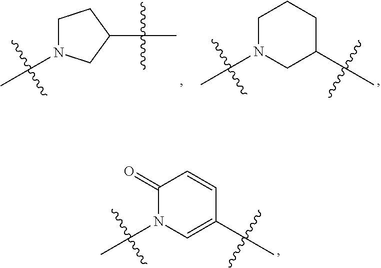 [SMILES: *C1CCN(*)C1 ; *C1CCCN(*)C1 ; *c1ccc(=O)n(*)c1]

each of which is optionally substituted with one or more R^{3a}. In some embodiments, Ring 3 is

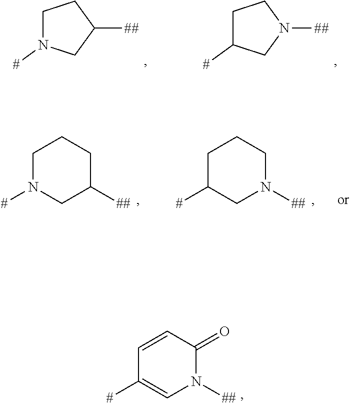

wherein # indicates attachment to the pyrimidine ring of Formula (I), and ## indicates attachment to Ring 4, when present.

In some embodiments of the compound of Formula (I), (II), (III), or (IV), or a related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), Ring 3 is phenyl optionally substituted with one or more R^{3b}. In some embodiments, Ring 3 is

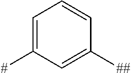

optionally substituted with one or more R^{3b}. In some embodiments, Ring 3 is

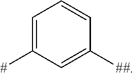

In some embodiments, Ring 3 is

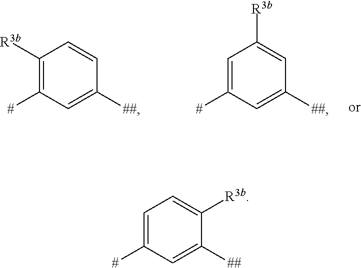

In some embodiments, each R^{3b} is independently halo, methyl, trifluoromethyl, OH, OCH₃, acetyl, or CN. In some embodiments, each R^{3b} is independently halo, methyl, or OCH₃.

In some embodiments of the compound of Formula (I), (II), (III), (IV), or any related formula such as (I-a), (I-b), (I-c), (J-d), (I-e), (I-f), (J-g), (I-h), (I-j), (I-k), (I-l), (I-m), (I-n), (I-o), (I-p), (I-q), (I-r), (I-s), (I-t), (I-u), (I-v), (I-w), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), Ring 3 is 5- to 6-membered heteroaryl optionally substituted with one or more R^{3b}. In some embodiments, Ring 3 is 5- to 6-membered nitrogen-containing heteroaryl optionally substituted with one or more R^{3b}. In some embodiments, Ring 3 is 6-membered heteroaryl optionally substituted with one or more R^{3b}. In some embodiments, Ring 3 is 5-membered heteroaryl optionally substituted with one or more R^{3b}. In some embodiments, Ring 3 is pyrazolyl optionally substituted with one or more R^{3b}. In some embodiments, Ring 3 is

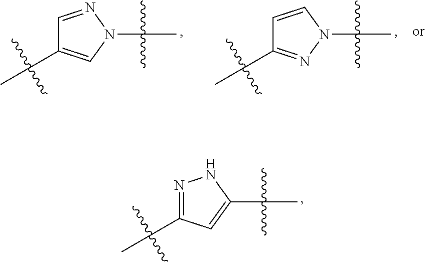 [SMILES: *c1cnn(*)c1 ; *c1ccn(*)n1 ; *c1cc(*)[nH]n1]

each of which is optionally substituted with one or more R^{3b}. In some embodiments, Ring 3 is

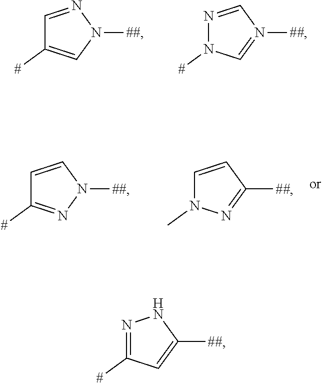

each of which is optionally substituted with one or more R^{3b}, wherein # indicates attachment to the pyrimidine ring of Formula (I), and ## indicates attachment to Ring 4, when present. In some embodiments, Ring 3 is

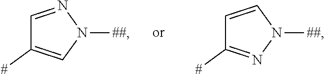

wherein # indicates attachment to the pyrimidine ring of Formula (I), and ## indicates attachment to Ring 4, when present. In some embodiments, Ring 3 is 6-membered heteroaryl optionally substituted with one or more R^{3b}. In some embodiments, Ring 3 is pyridinyl, pyrimidinyl, or pyrazinyl optionally substituted with one or more R^{3b}.

In some embodiments, Ring 3 is

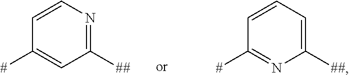

each of which is optionally substituted with one or more R^{3b}, wherein # indicates attachment to the pyrimidine ring of Formula (I), and ## indicates attachment to Ring 4, when present.

In some embodiments of the compound of Formula (I), (II), (III), (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), Ring 3 is other than 5-membered heteroaryl, and Ring 4 is absent. In some such embodiments, Ring 3 is 3- to 6-membered cycloalkyl optionally substituted with one or more R^{3a}; 4- to 6-membered heterocyclyl optionally substituted with one or more R^{3a}; phenyl optionally substituted with one or more R^{3b}; or 6-membered heteroaryl optionally substituted with one or more R^{3b}, and Ring 4 is absent. In some such embodiments, Ring 3 is 3- to 6-membered cycloalkyl optionally substituted with one or more R^{3a}; 4- to 6-membered heterocyclyl optionally substituted with one or more R^{3a}; or phenyl optionally substituted with one or more R^{3b}; and Ring 4 is absent.

In some embodiments of the compound of Formula (I), (II), (III), (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), Ring 4 is present. In some embodiments, Ring 4 is present and is unsubstituted. In some embodiments, Ring 4 is present and is unsubstituted or substituted with halo. In some embodiments, Ring 4 is phenyl substituted with halo. In some embodiments, Ring 4 is phenyl substituted with fluoro.

In some embodiments of the compound of Formula (I), (II), (III), (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), Ring 4 is present and is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b}, wherein each R^{4a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₐN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo, and each R^{4b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)~R^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x}, wherein when Ring 3 is 5-membered heteroaryl, Ring 4 is not cycloalkyl or tetrahydropyranyl. In some embodiments, each R^{4a} is independently halo, methyl, trifluoromethyl, OH, OCH₃, acetyl, CN, or oxo. In some embodiments, each R^{4a} is methyl or acetyl. In some embodiments, each R^{4b} is independently halo, methyl, trifluoromethyl, OH, OCH₃, acetyl, or CN.

In some embodiments of the compound of Formula (I), (II), (III), (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), Ring 4 is 3- to 6-membered cycloalkyl optionally substituted with one or more R^{4a} and Ring 3 is not 5-membered heteroaryl. In some embodiments, Ring 4 is 4- to 6-membered cycloalkyl optionally substituted with one or more R^{4a}, and Ring 3 is not 5-membered heteroaryl. In some embodiments, Ring 4 is cyclopropyl, cyclobutyl, cyclopentyl, or cyclohexyl optionally substituted with one or more R^{4a}, and Ring 3 is not 5-membered heteroaryl. In some embodiments, Ring 4 is cyclopropyl, cyclobutyl, cyclopentyl, or cyclohexyl, and Ring e is not 5-membered heteroaryl. In some embodiments, Ring 4 is cyclobutyl, cyclopentyl, or cyclohexyl, and Ring 3 is not 5-membered heteroaryl.

In some embodiments of the compound of Formula (I), (II), (III), (IV), or any related formula such as ((I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), Ring 4 is 4- to 6-membered heterocyclyl optionally substituted with one or more R^{4a}, wherein when Ring 3 is 5-membered heteroaryl, Ring 4 is not tetrahydropyranyl. In some embodiments, Ring 4 is 4- to 6-membered nitrogen-containing heterocyclyl optionally substituted with one or more R^{4a}. In some embodiments, Ring 4 is 5- to 6-membered nitrogen-containing heterocyclyl optionally substituted with one or more R^{4a}. In some embodiments, Ring 4 is pyrrolidinyl, piperidinyl, or piperazinyl, each of which is optionally substituted with one or more R^{4a}. In some embodiments, Ring 4 is pyrrolidinyl or piperidinyl, each of which is optionally substituted with one or more R^{4a}. In some embodiments, Ring 4 is

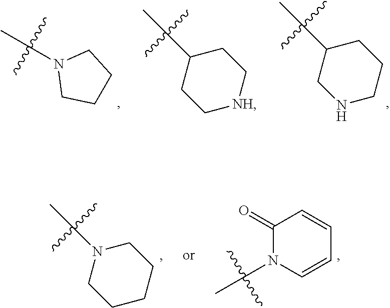 [SMILES: *N1CCCC1 ; *C1CCNCC1 ; *C1CCCNC1 ; *N1CCCCC1 ; *n1ccccc1=O]

each of which is optionally substituted with one or more R^{4a}. In some embodiments, Ring 4 is

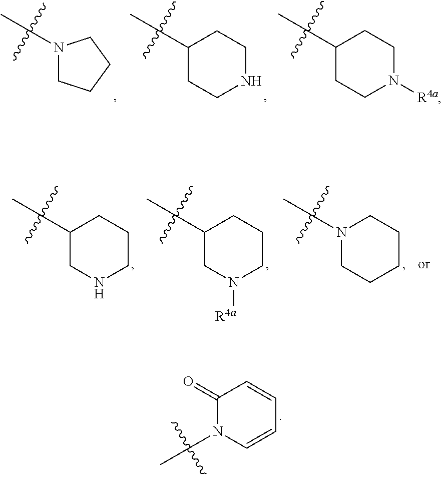

In some embodiments, each R^{4a} is independently halo, methyl, trifluoromethyl, OH, OCH₃, acetyl, CN, or oxo. In some embodiments, each R^{4a} is methyl or acetyl.

In some embodiments of the compound of Formula (I), (II), (III), (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), Ring 4 is phenyl optionally substituted with one or more R^{4b}. In some embodiments, Ring 4 is

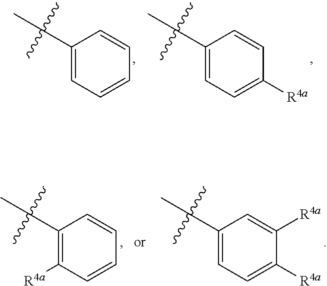

In some embodiments, each R^{4a} is independently halo, methyl, trifluoromethyl, OH, OCH₃, acetyl, or CN. In some embodiments, each R^{4a} is halo, methyl, or OCH₃.

In some embodiments of the compound of Formula (I), (II), (III), (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), Ring 4 is 5- to 6-membered heteroaryl optionally substituted with one or more R^{4b}. In some embodiments, Ring 4 is 5- to 6-membered nitrogen-containing heteroaryl optionally substituted with one or more R^{4b}. In some embodiments, Ring 4 is 6-membered heteroaryl optionally substituted with one or more R^{4b}. In some embodiments, Ring 4 is pyridinyl or pyrazinyl, each of which is optionally substituted with one or more R^{4b}. In some embodiments, Ring 4 is

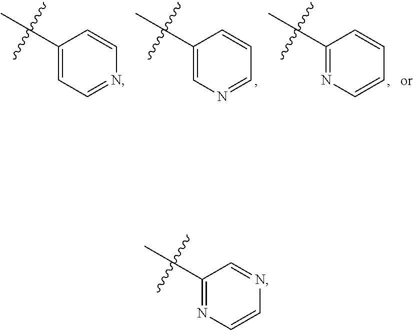 [SMILES: *c1ccncc1 ; *c1cccnc1 ; *c1ccccn1 ; *c1cnccn1]

each of which is optionally substituted with one or more R^{4b}. In some embodiments, Ring 4 is

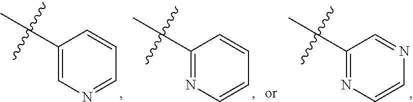 [SMILES: *c1cccnc1 ; *c1ccccn1 ; *c1cnccn1]

each of which is optionally substituted with one or more R^{4b}. In some embodiments, Ring 4 is

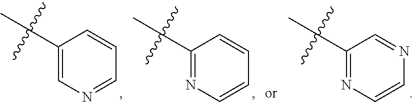 [SMILES: *c1cccnc1 ; *c1ccccn1 ; *c1cnccn1]

In some embodiments, Ring 4 is

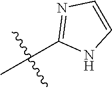 [SMILES: *c1ncc[nH]1]

optionally substituted with one or more R^{4b}.

In some embodiments, Ring 4 is

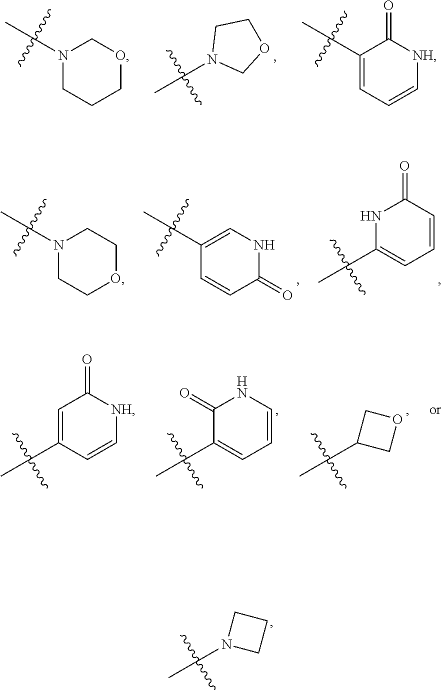

each of which is optionally substituted with one or more R^{4a}.

In some embodiments, Ring 4 is

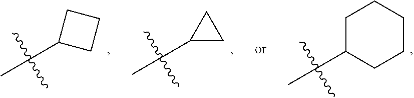

each of which is optionally substituted with one or more R^{4a}.

In some embodiments of the compound of Formula (I), (II), (III), (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), Ring 3 is 3- to 6-membered cycloalkyl optionally substituted with one or more R^{3a}, 4- to 6-membered heterocyclyl optionally substituted with one or more R^{3a}, phenyl optionally substituted with one or more R^{3b}, or 6-membered heteroaryl optionally substituted with one or more R^{3b}; and Ring 4 is absent. In some embodiments of the compound of Formula (I), Ring 3 is 3- to 6-membered cycloalkyl optionally substituted with one or more R^{3a}, 4- to 6-membered heterocyclyl optionally substituted with one or more R^{3a}, or phenyl optionally substituted with one or more R^{3b}; and Ring 4 is absent.

In some embodiments of the compound of Formula (I), (TT), (III), (IV), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), Ring 3 is 5-membered heteroaryl, and Ring 4 is present and is not cycloalkyl or tetrahydropyranyl. In some embodiments of the compound of Formula (I), Ring 3 is 5-membered heteroaryl, and Ring 4 is present and is 4- to 6-membered nitrogen-containing heterocyclyl optionally substituted with one or more R^{4a}, phenyl optionally substituted with one or more R^{4b}, or 5- to 6-membered heteroaryl optionally substituted with one or more R^{4b}. In some embodiments of the compound of Formula (I), Ring 3 is 5-membered heteroaryl, and Ring 4 is present and is phenyl optionally substituted with one or more R^{4b}, or 5- to 6-membered heteroaryl optionally substituted with one or more R^{4b}.

In some embodiments, Ring 3 is phenyl, and Ring 4 is present and is 4- to 6-membered nitrogen-containing heterocyclyl optionally substituted with one or more R^{4a} phenyl optionally substituted with one or more R^{4b}, or 5- to 6-membered heteroaryl optionally substituted with one or more R^{4b}. In some embodiments, Ring 3 is phenyl and Ring 4 is present and is 4- to 6-membered nitrogen-containing heterocyclyl optionally substituted with one or more R^{4a}. In some embodiments, Ring 3 is phenyl and Ring 4 is present and is

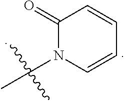 [SMILES: *n1ccccc1=O]

In some embodiments of the compound of Formula (I), or any related formula such as (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), Ring 4 is absent and Q is of Formula (i). In some embodiments, Ring 4 is present and Q is of Formula (i).

In some embodiments, Ring 4 is absent. In some embodiments, Ring 4 is present.

In some embodiments of the compound of Formula (I), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (IV-a), (IV-a), (IV-c), (IV-d), (IV-e), (IV-f), (IV-g), (IV-h), (IV-j), (IV-k), or (IV-l), Ring A, X, Ring B, W, and C together are:

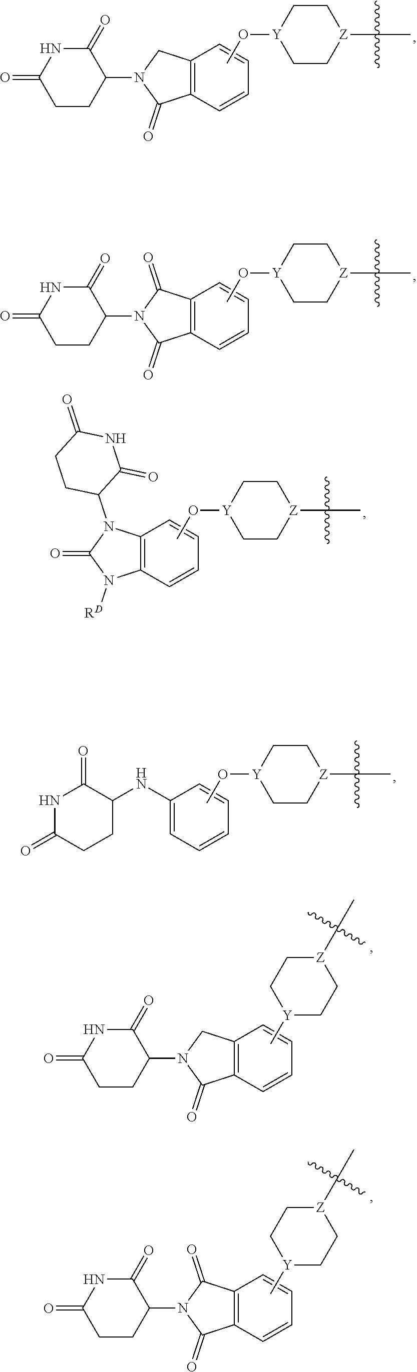

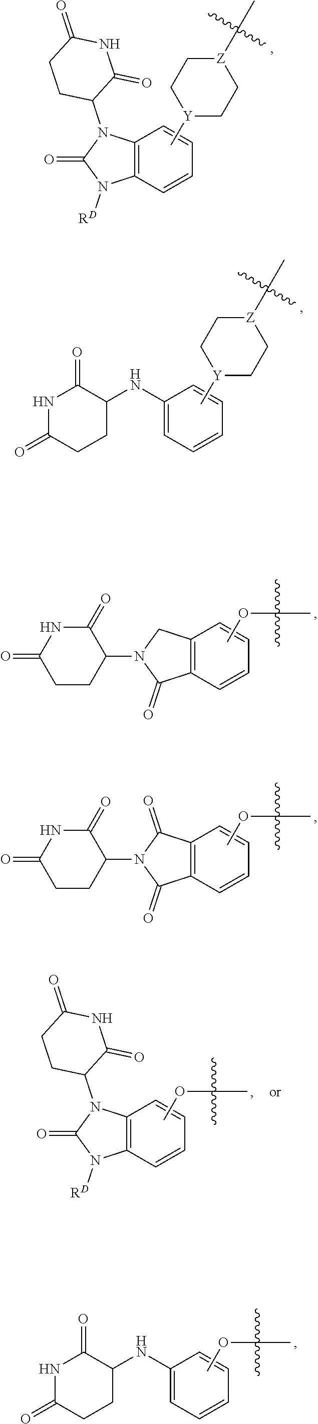

wherein each

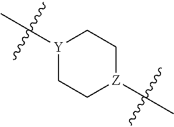

is optionally substituted with one or more R^{C}, and wherein Y and Z are independently CH or N. In some embodiments, Ring A, X, Rin B, W, and C together are:

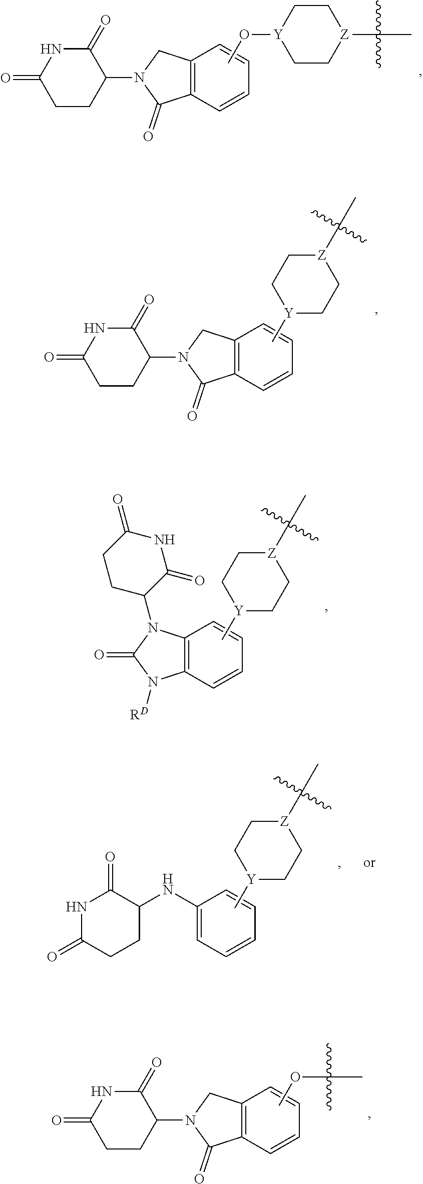

wherein each

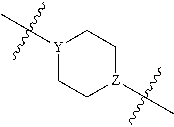

is optionally substituted with one or more R^{C}, and wherein Y and Z are independently CH or N. In some embodiments, Ring A, X, Ring B, W, and C together are:

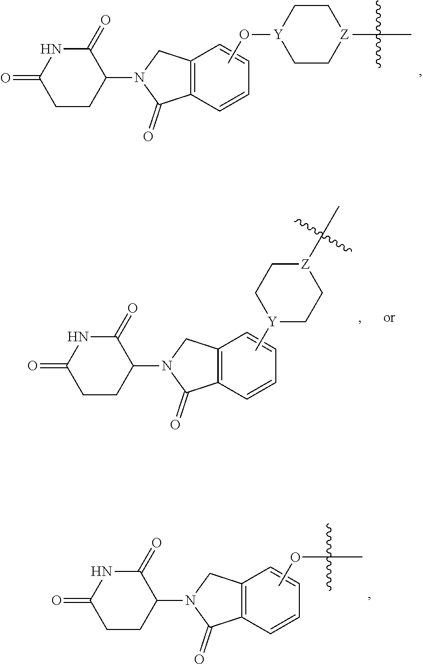

wherein each

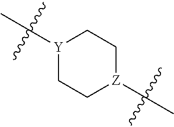

is optionally substituted with one or more R^{C}, and wherein Y and Z are independently CH or N. In some embodiments, Ring A, X, Ring B, W, and C together are:

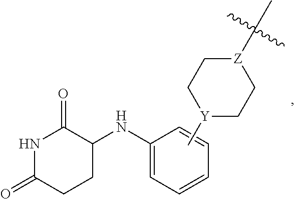

wherein each

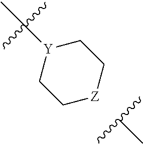

is optionally substituted with one or more R^{C}, and wherein Y and Z are independently CH or N.

In some embodiments, Ring A, X, Ring B, W, and C together are:

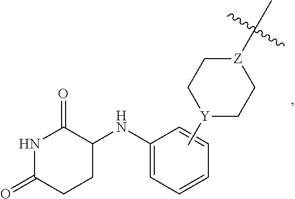

wherein

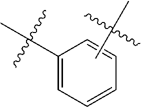

is optionally substituted with R^{2b}, wherein each

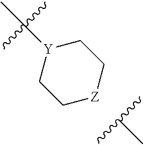

is optionally substituted with one or more R^{C}, and wherein Y and Z are independently CH or N.

In some embodiments, when Ring 3 is 5-membered heteroaryl, Ring 4 is present and is not cycloalkyl or tetrahydropyranyl; and when Ring 1 is 8-membered heterocyclyl, Ring 3 is phenyl substituted with CN, Ring 4 is absent, Q is R^{Q}, and V is —OC(O)#, then R^{Q} is not tert-butyl. In some embodiments, when Ring 3 is 5-membered heteroaryl, Ring 4 is present and is not cycloalkyl or tetrahydropyranyl. In some embodiments, when Ring 1 is 8-membered heterocyclyl, Ring 3 is phenyl substituted with CN, Ring 4 is absent, Q is R^{Q}, and V is —OC(O)#, then R^{Q} is not tert-butyl.

It is to be understood that all embodiments of Formula (I) and sub-formulas thereof, also apply to Formulas (II), (III), and (IV), including all sub-formulas, and variations thereof, as applicable. Further, the present disclosure contemplates combinations of features as disclosed herein, as if each were individually listed. Exemplary combinations of features are provided herein but are not to be construed as limiting.

It is to be understood that all embodiments of Formula (I) and sub-formulas thereof, also apply to Formulas (I′), (II), (III), and (IV), including all sub-formulas, and variations thereof, as applicable. Further, the present disclosure contemplates combinations of features as disclosed herein, as if each were individually listed. Exemplary combinations of features are provided herein but are not to be construed as limiting.

Representative compounds are listed in Table 1 and Table 2.

In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from Table 1 or Table 2. In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from Table 1. In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from Table 2.

In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 1-43 and 91 of Table 1, and compounds 44-90 of Table 2. In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 1-43 and 91-274 of Table 1, and compounds 44-90 and 275-605 of Table 2. In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 1-43, 91-274, and 606-631 of Table 1, and compounds 44-90, 275-605, and 632-705 of Table 2. In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 1-43 and 91 of Table 1. In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 1-43 and 91-274 of Table 1. In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 1-43, 91-274, and 606-631 of Table 1. In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 44-90 of Table 2. In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 44-90 and 275-605 of Table 2. In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 44-90, 275-605, and 632-705 of Table 2.

TABLE 1
Representative Compounds of Formula (I′), (I), (II), and (III)

| Cmpd. | | |
|---|---|---|
| No. | Structure | Name |
| 1 | 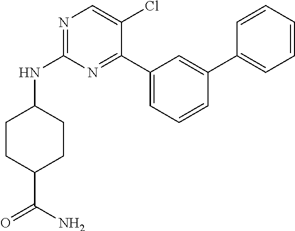 [SMILES: NC(=O)C1CCC(Nc2ncc(Cl)c(-c3cccc(-c4ccccc4)c3)n2)CC1] | 4-((4-([1,1′-biphenyl]-3- yl)-5-chloropyrimidin-2- yl)amino)cyclohexane-1- carboxamide |
| 2 | 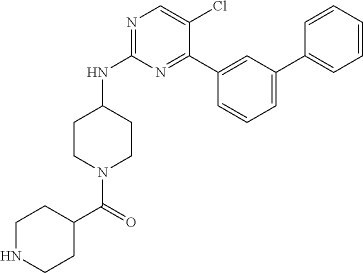 [SMILES: O=C(C1CCNCC1)N1CCC(Nc2ncc(Cl)c(-c3cccc(-c4ccccc4)c3)n2)CC1] | (4-((4-([1,1′-biphenyl]-3- yl)-5-chloropyrimidin-2- yl)amino)piperidin-1- yl)(piperidin-4- yl)methanone |
| 3 | 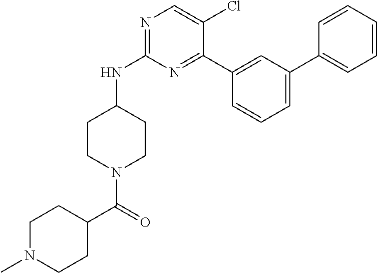 [SMILES: CN1CCC(C(=O)N2CCC(Nc3ncc(Cl)c(-c4cccc(-c5ccccc5)c4)n3)CC2)CC1] | (4-((4-([1,1′-biphenyl]-3- yl)-5-chloropyrimidin-2- yl)amino)piperidin-1-yl)(1- methylpiperidin-4- yl)methanone |
| 4 | 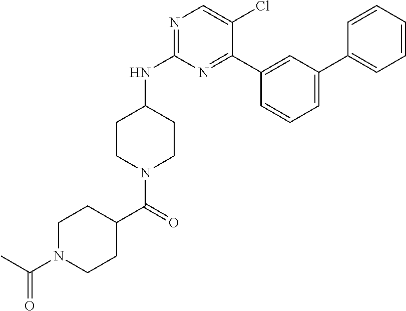 [SMILES: CC(=O)N1CCC(C(=O)N2CCC(Nc3ncc(Cl)c(-c4cccc(-c5ccccc5)c4)n3)CC2)CC1] | 1-(4-(4-((4-([1,1′- biphenyl]-3-yl)-5- choropyrimidin-2- yl)amino)piperidine-1- carbonyl)piperidin-1- yl)ethan-1-one |
| 5 | 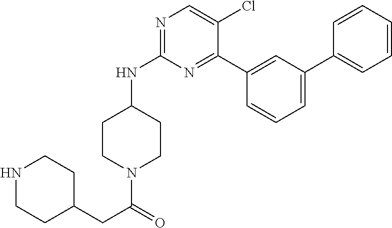 [SMILES: O=C(CC1CCNCC1)N1CCC(Nc2ncc(Cl)c(-c3cccc(-c4ccccc4)c3)n2)CC1] | 1-(4-((4-([1,1′-biphenyl]-3- yl)-5-chloropyrimidin-2- yl)amino)piperidin-1-yl)-2- (piperidin-4-yl)ethan-1-one |
| 6 | 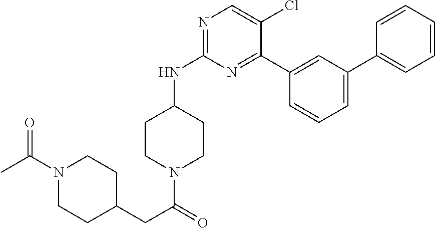 [SMILES: CC(=O)N1CCC(CC(=O)N2CCC(Nc3ncc(Cl)c(-c4cccc(-c5ccccc5)c4)n3)CC2)CC1] | 1-(4-((4-([1,1′-biphenyl]-3- yl)-5-chloropyrimidin-2- yl)amino)piperidin-1-yl)-2- (1-acetylpiperidin-4- yl)ethan-1-one |
| 7 | 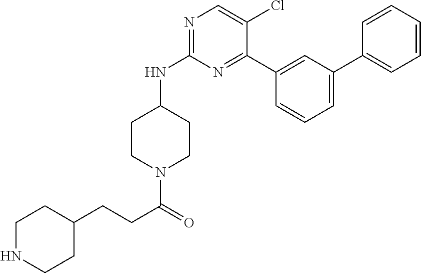 [SMILES: O=C(CCC1CCNCC1)N1CCC(Nc2ncc(Cl)c(-c3cccc(-c4ccccc4)c3)n2)CC1] | 1-(4-((4-([1,1′-biphenyl]-3- yl)-5-chloropyrimidin-2- yl)amino)piperidin-1-yl)-3- (piperidin-4-yl)propan-1- one |
| 8 | 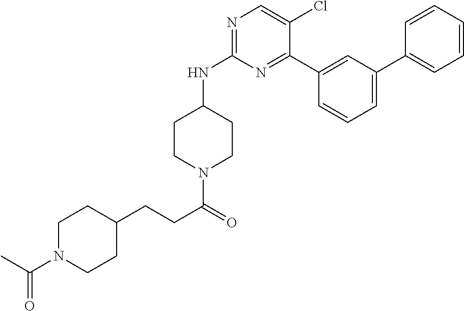 [SMILES: CC(=O)N1CCC(CCC(=O)N2CCC(Nc3ncc(Cl)c(-c4cccc(-c5ccccc5)c4)n3)CC2)CC1] | 1-(4-((4-([1,1′-biphenyl]-3- yl)-5-chloropyrimidin-2- yl)amino)piperidin-1-yl)-3- (1-acetylpiperidin-4- yl)propan-1-one |
| 9 | 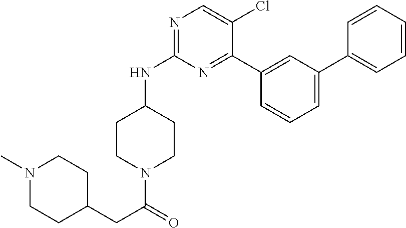 [SMILES: CN1CCC(CC(=O)N2CCC(Nc3ncc(Cl)c(-c4cccc(-c5ccccc5)c4)n3)CC2)CC1] | 1-(4-((4-([1,1′-biphenyl]-3- yl)-5-chloropyrimidin-2- yl)amino)piperidin-1-yl)-2- (1-methylpiperidin-4- yl)ethan-1-one |
| 10 | 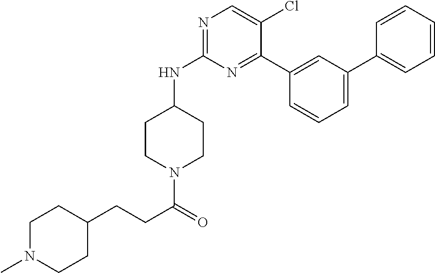 [SMILES: CN1CCC(CCC(=O)N2CCC(Nc3ncc(Cl)c(-c4cccc(-c5ccccc5)c4)n3)CC2)CC1] | 1-(4-((4-([1,1′-biphenyl]-3- yl)-5-chloropyrimidin-2- yl)amino)piperidin-1-yl)-3- (1-methylpiperidin-4- yl)propan-1-one |
| 11 | 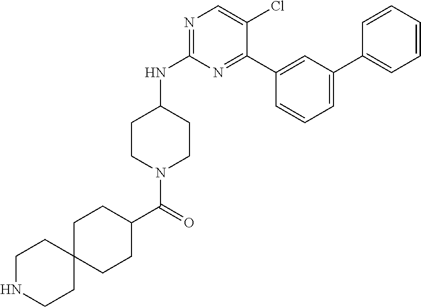 [SMILES: O=C(C1CCC2(CCNCC2)CC1)N1CCC(Nc2ncc(Cl)c(-c3cccc(-c4ccccc4)c3)n2)CC1] | (4-((4-([1,1′-biphenyl]-3- yl)-5-chloropyrimidin-2- yl)amino)piperidin-1-yl)(3- azaspiro[5.5]undecan-9- yl)methanone |
| 12 | 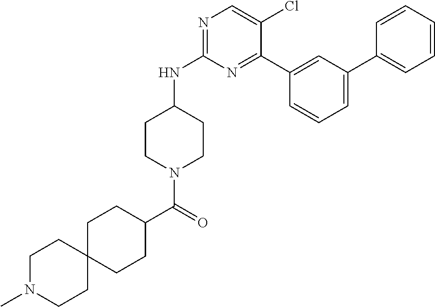 [SMILES: CN1CCC2(CCC(C(=O)N3CCC(Nc4ncc(Cl)c(-c5cccc(-c6ccccc6)c5)n4)CC3)CC2)CC1] | (4-((4-([1,1′-biphenyl]-3- yl)-5-chloropyrimidin-2- yl)amino)piperidin-1-yl)(3- methyl-3- azaspiro[5.5]undecan-9- yl)methanone |
| 13 | 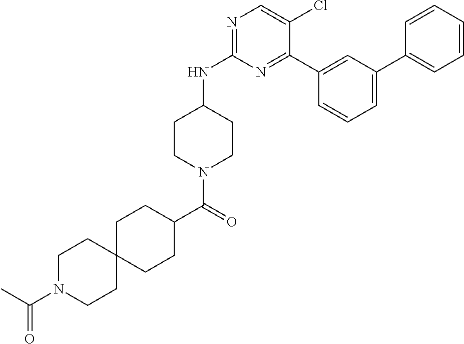 [SMILES: CC(=O)N1CCC2(CCC(C(=O)N3CCC(Nc4ncc(Cl)c(-c5cccc(-c6ccccc6)c5)n4)CC3)CC2)CC1] | 1-(9-(4-((4-([′,1′- biphenyl]-3-yl)-5- chloropyrimidin-2- yl)amino)piperidine-1- carbonyl)-3- azaspiro[5.5]undecan-3- yl)ethan-1-one |
| 14 | 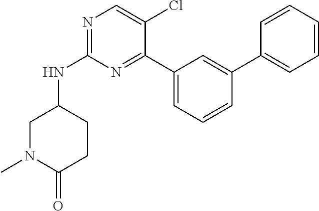 [SMILES: CN1CC(Nc2ncc(Cl)c(-c3cccc(-c4ccccc4)c3)n2)CCC1=O] | 5-((4-([1,1′-biphenyl]-3- yl)-5-chloropyrimidin-2- yl)amino)-1- methylpiperidin-2-one |
| 15 | 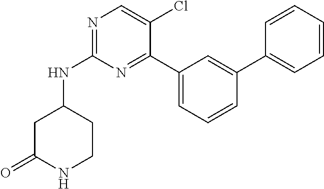 [SMILES: O=C1CC(Nc2ncc(Cl)c(-c3cccc(-c4ccccc4)c3)n2)CCN1] | 4-((4-([1,1′-biphenyl]-3- yl)-5-chloropyrimidin-2- yl)amino)piperidin-2-one |
| 16 | 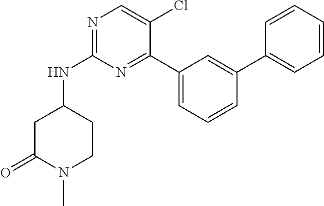 [SMILES: CN1CCC(Nc2ncc(Cl)c(-c3cccc(-c4ccccc4)c3)n2)CC1=O] | 4-((4-([1,1′-biphenyl]-3- yl)-5-chloropyrimidin-2- yl)amino)-1- methylpiperidin-2-one |
| 17 | 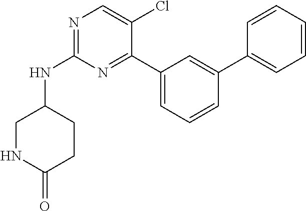 [SMILES: O=C1CCC(Nc2ncc(Cl)c(-c3cccc(-c4ccccc4)c3)n2)CN1] | 5-((4-([1,1′-biphenyl]-3- yl)-5-chloropyrimidin-2- yl)amino)piperidin-2-one |
| 18 | 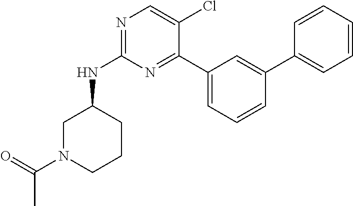 [SMILES: CC(=O)N1CCC[C@H](Nc2ncc(Cl)c(-c3cccc(-c4ccccc4)c3)n2)C1] | (S)-1-(3-((4-([1,1′- biphenyl]-3-yl)-5- chloropyrimidin-2- yl)amino)piperidin-1- yl)ethan-1-one |
| 19 | 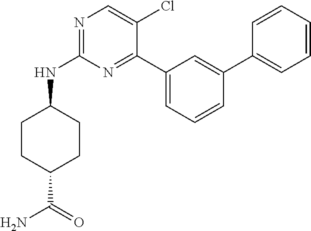 [SMILES: NC(=O)[C@H]1CC[C@H](Nc2ncc(Cl)c(-c3cccc(-c4ccccc4)c3)n2)CC1] | trans-4-((4-([1,1′- biphenyl]-3-yl)-5- chloropyrimidin-2- yl)amino)cyclohexane-1- carboxamide |
| 20 | 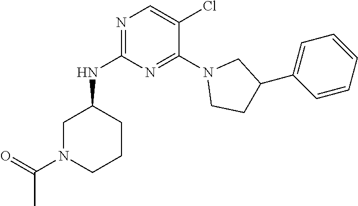 [SMILES: CC(=O)N1CCC[C@H](Nc2ncc(Cl)c(N3CCC(c4ccccc4)C3)n2)C1] | 1-((3S)-3-((5-chloro-4-(3- phenylpyrrolidin-1- yl)pyrimidin-2- yl)amino)piperidin-1- yl)ethan-1-one |
| 21 | 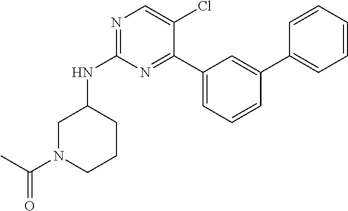 [SMILES: CC(=O)N1CCCC(Nc2ncc(Cl)c(-c3cccc(-c4ccccc4)c3)n2)C1] | 1-(3-((4-([1,1′-biphenyl]-3- yl)-5-chloropyrimidin-2- yl)amino)piperidin-1- yl)ethan-1-one |
| 22 | 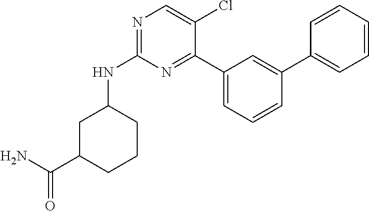 [SMILES: NC(=O)C1CCCC(Nc2ncc(Cl)c(-c3cccc(-c4ccccc4)c3)n2)C1] | 3-((4-([1,1′-biphenyl]-3- yl)-5-chloropyrimidin-2- yl)amino)cyclohexane-1- carboxamide |
| 23 | 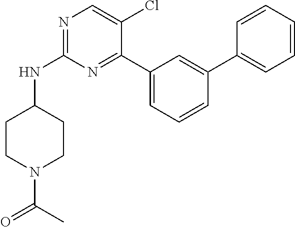 [SMILES: CC(=O)N1CCC(Nc2ncc(Cl)c(-c3cccc(-c4ccccc4)c3)n2)CC1] | 1-(4-((4-[(1,1′-biphenyl]-3- yl)-5-chloropyrimidin-2- yl)amino)piperidin-1- yl)ethan-1-one |
| 24 | 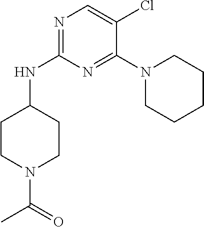 [SMILES: CC(=O)N1CCC(Nc2ncc(Cl)c(N3CCCCC3)n2)CC1] | 1-(4-((5-chloro-4- (piperidin-1-yl)pyrimidin- 2-yl)amino)piperidin-1- yl)ethan-1-one |
| 25 | 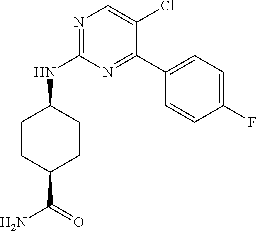 [SMILES: NC(=O)[C@H]1CC[C@@H](Nc2ncc(Cl)c(-c3ccc(F)cc3)n2)CC1] | cis-4-((5-chloro-4-(4- fluorophenyl)pyrimidin-2- yl)amino)cyclohexane-1- carboxamide |
| 26 | 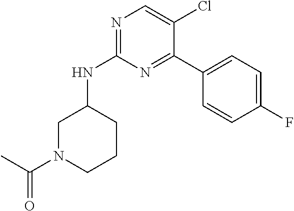 [SMILES: CC(=O)N1CCCC(Nc2ncc(Cl)c(-c3ccc(F)cc3)n2)C1] | 1-(3-((5-chloro-4-(4- fluorophenyl)pyrimidin-2- yl)amino)piperidin-1- yl)ethan-1-one |
| 27 | 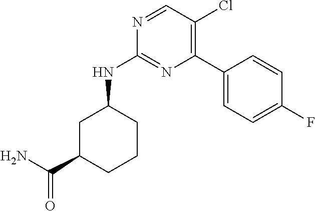 [SMILES: NC(=O)[C@@H]1CCC[C@H](Nc2ncc(Cl)c(-c3ccc(F)cc3)n2)C1] | 1-(4-((5-chloro-4-(4- fluorophenyl)pyrimidin-2- yl)amino)piperidin-1- yl)ethan-1-one |
| 28 | 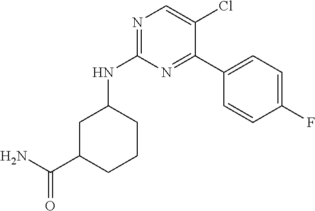 [SMILES: NC(=O)C1CCCC(Nc2ncc(Cl)c(-c3ccc(F)cc3)n2)C1] | trans-3-((5-chloro-4-(4- fluorophenyl)pyrimidin-2- yl)amino)cyclohexane-1- carboxamide |
| 29 | 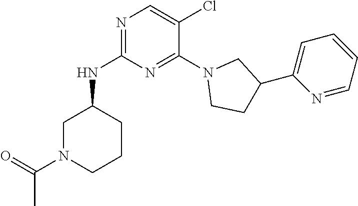 [SMILES: CC(=O)N1CCC[C@H](Nc2ncc(Cl)c(N3CCC(c4ccccn4)C3)n2)C1] | 1-((3S)-3-((5-chloro-4-(3- (pyridin-2-yl)pyrrolidin-1- yl)pyrimidin-2- yl)amino)piperidin-1- yl)ethan-1-one |
| 30 | 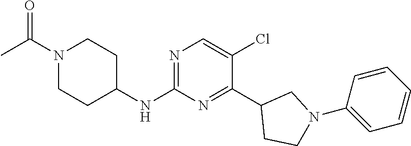 [SMILES: CC(=O)N1CCC(Nc2ncc(Cl)c(C3CCN(c4ccccc4)C3)n2)CC1] | 1-(4-((5-chloro-4-(1- phenylpyrrolidin-3- yl)pyrimidin-2- yl)amino)piperidin-1- yl)ethan-1-one |
| 31 | 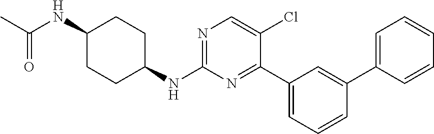 [SMILES: CC(=O)N[C@H]1CC[C@@H](Nc2ncc(Cl)c(-c3cccc(-c4ccccc4)c3)n2)CC1] | cis-N-(4-((4-([1,1′- biphenyl]-3-yl)-5- chloropyrimidin-2- yl)amino)cyclohexyl) acetamide |
| 32 | 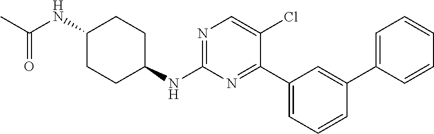 [SMILES: CC(=O)N[C@H]1CC[C@H](Nc2ncc(Cl)c(-c3cccc(-c4ccccc4)c3)n2)CC1] | trans-N-(4-((4-([1,1′- biphenyl]-3-yl)-5- chloropyrimidin-2- yl)amino)cyclohexyl) acetamide |
| 33 | 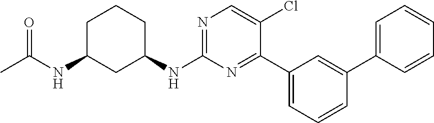 [SMILES: CC(=O)N[C@H]1CCC[C@@H](Nc2ncc(Cl)c(-c3cccc(-c4ccccc4)c3)n2)C1] | cis-N-(3-((4-([1,1′- biphenyl]-3-yl)-5- chloropyrimidin-2- yl)amino)cyclohexyl) acetamide |
| 34 | 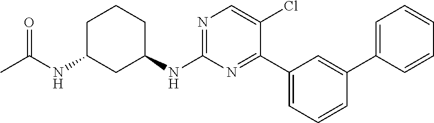 [SMILES: CC(=O)N[C@@H]1CCC[C@@H](Nc2ncc(Cl)c(-c3cccc(-c4ccccc4)c3)n2)C1] | trans-N-(3-((4-([1,1′- biphenyl]-3-yl)-5- chloropyrimidin-2- yl)amino)cyclohexyl) acetamide |
| 35 | 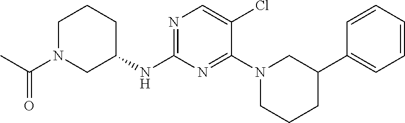 [SMILES: CC(=O)N1CCC[C@H](Nc2ncc(Cl)c(N3CCCC(c4ccccc4)C3)n2)C1] | 1-((3S)-3-((5-chloro-4-(3- phenylpiperidin-1-yl) pyrimidin-2-yl) amino)piperidin-1-yl) ethan-1-one |
| 36 | 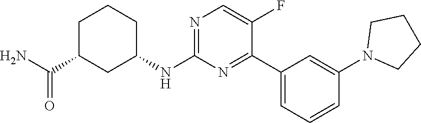 [SMILES: NC(=O)[C@@H]1CCC[C@H](Nc2ncc(F)c(-c3cccc(N4CCCC4)c3)n2)C1] | cis-3-((5-fluoro-4-(3- (pyrrolidin-1- yl)phenyl)pyrimidin-2- yl)amino)cyclohexane-1- carboxamide |
| 37 | 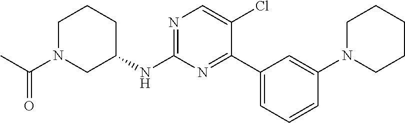 [SMILES: CC(=O)N1CCC[C@H](Nc2ncc(Cl)c(-c3cccc(N4CCCCC4)c3)n2)C1] | (S)-1-(3-((5-chloro-4-(3- (piperidin-1- yl)phenyl)pyrimidin-2- yl)amino)piperidin-1- yl)ethan-1-one |
| 38 | 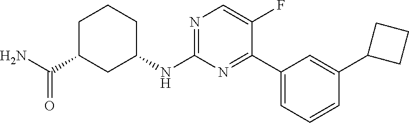 [SMILES: NC(=O)[C@@H]1CCC[C@H](Nc2ncc(F)c(-c3cccc(C4CCC4)c3)n2)C1] | cis-3-((4-(3- cyclobutylphenyl)-5- fluoropyrimidin-2- yl)amino)cyclohexane-1- carboxamide |
| 39 | 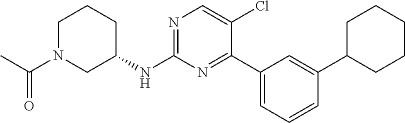 [SMILES: CC(=O)N1CCC[C@H](Nc2ncc(Cl)c(-c3cccc(C4CCCCC4)c3)n2)C1] | (S)-1-(3-((5-chloro-4-(3- cyclohexylphenyl) pyrimidin-2-yl)amino) piperidin-1-yl)ethan-1-one |
| 40 | 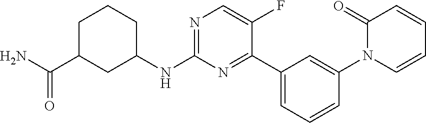 [SMILES: NC(=O)C1CCCC(Nc2ncc(F)c(-c3cccc(-n4ccccc4=O)c3)n2)C1] | 3-[[5-fluoro-4-[3-(2-oxo-1- pyridyl)phenyl]pyrimidin- 2-yl]amino]cyclohexane- carboxamide |
| 41 | 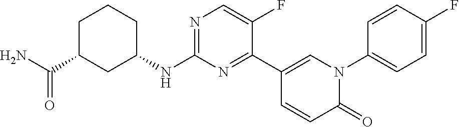 [SMILES: NC(=O)[C@@H]1CCC[C@H](Nc2ncc(F)c(-c3ccc(=O)n(-c4ccc(F)cc4)c3)n2)C1] | cis-3-((5-fluoro-4-(1-(4- fluorophenyl)-6-oxo-1,6- dihydropyridin-3- yl)pyrimidin-2- yl)amino)cyclohexane-1- carboxamide |
| 42 | 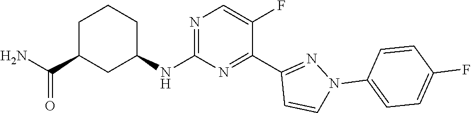 [SMILES: NC(=O)[C@H]1CCC[C@@H](Nc2ncc(F)c(-c3ccn(-c4ccc(F)cc4)n3)n2)C1] | cis-3-((5-fluoro-4-(1-(4- fluorophenyl)-1H-pyrazol- 3-yl)pyrimidin-2- yl)amino)cyclohexane-1- carboxamide |
| 43 | 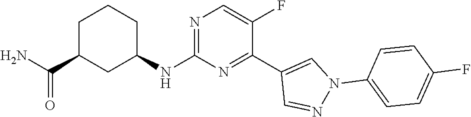 [SMILES: NC(=O)[C@H]1CCC[C@@H](Nc2ncc(F)c(-c3cnn(-c4ccc(F)cc4)c3)n2)C1] | cis-3-((5-fluoro-4-(1-(4- fluorophenyl)-1H-pyrazol- 4-yl)pyrimidin-2- yl)amino)cyclohexane-1- carboxamide |
| 91 | 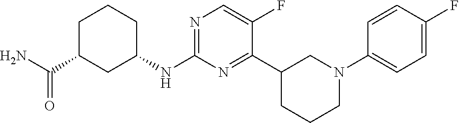 [SMILES: NC(=O)[C@@H]1CCC[C@H](Nc2ncc(F)c(C3CCCN(c4ccc(F)cc4)C3)n2)C1] | cis-3-((5-fluoro-4-(1-(4- fluorophenyl)piperidin-3- yl)pyrimidin-2- yl)amino)cyclohexane-1- carboxamide |
| 92 | 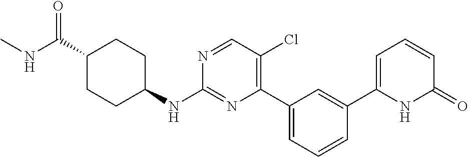 [SMILES: CNC(=O)[C@H]1CC[C@H](Nc2ncc(Cl)c(-c3cccc(-c4cccc(=O)[nH]4)c3)n2)CC1] | trans-(1r,4r)-4-((5-chloro- 4-(3-(6-oxo-1,6- dihydropyridin-2- yl)phenyl)pyrimidin-2- yl)amino)-N- methylcyclohexane-1- carboxamide |
| 93 | 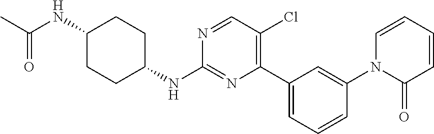 [SMILES: CC(=O)N[C@H]1CC[C@@H](Nc2ncc(Cl)c(-c3cccc(-n4ccccc4=O)c3)n2)CC1] | cis-N-((1s,4s)-4-((5-chloro- 4-(3-(2-oxopyridin-1(2H)- yl)phenyl)pyrimidin-2- yl)amino)cyclohexyl) acetamide |
| 94 | 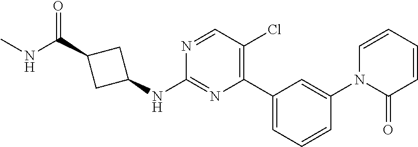 [SMILES: CNC(=O)[C@H]1C[C@@H](Nc2ncc(Cl)c(-c3cccc(-n4ccccc4=O)c3)n2)C1] | cis-3-((5-chloro-4-(3-(2- oxopyridin-1(2H)- yl)phenyl)pyrimidin-2- yl)amino)-N- methylcyclobutane-1- carboxamide |
| 95 | 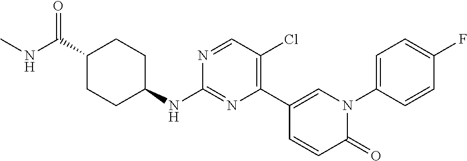 [SMILES: CNC(=O)[C@H]1CC[C@H](Nc2ncc(Cl)c(-c3ccc(=O)n(-c4ccc(F)cc4)c3)n2)CC1] | trans-(1r,4r)-4-((5-chloro- 4-(1-(4-fluorophenyl)-6- oxo-1,6-dihydropyridin-3- yl)pyrimidin-2-yl)amino)- N-methylcyclohexane-1- carboxamide |
| 96 | 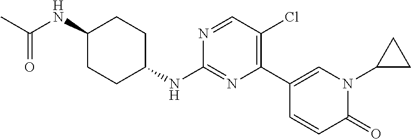 [SMILES: CC(=O)N[C@H]1CC[C@H](Nc2ncc(Cl)c(-c3ccc(=O)n(C4CC4)c3)n2)CC1] | trans-N-((1r,4r)-4-((5- chloro-4-(1-cyclopropyl-6- oxo-1,6-dihydropyridin-3- yl)pyrimidin-2- yl)amino)cyclohexyl) acetamide |
| 97 | 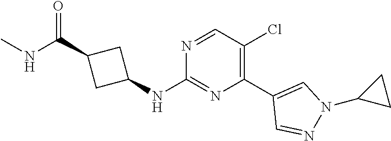 [SMILES: CNC(=O)[C@H]1C[C@@H](Nc2ncc(Cl)c(-c3cnn(C4CC4)c3)n2)C1] | cis-3-((5-chloro-4-(1- cyclopropyl-1H-pyrazol-4- yl)pyrimidin-2-yl)amino)- N-methylcyclobutane-1- carboxamide |
| 98 | 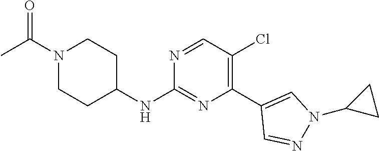 [SMILES: CC(=O)N1CCC(Nc2ncc(Cl)c(-c3cnn(C4CC4)c3)n2)CC1] | 1-(4-((5-chloro-4-(1- cyclopropyl-1H-pyrazol-4- yl)pyrimidin-2- yl)amino)piperidin-1- yl)ethan-1-one |
| 99 | 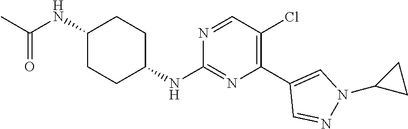 [SMILES: CC(=O)N[C@H]1CC[C@@H](Nc2ncc(Cl)c(-c3cnn(C4CC4)c3)n2)CC1] | cis-N-((1s,4s)-4-((5-chloro- 4-(1-cyclopropyl-1H- pyrazol-4-yl)pyrimidin-2- yl)amino)cyclohexyl) acetamide |
| 100 | 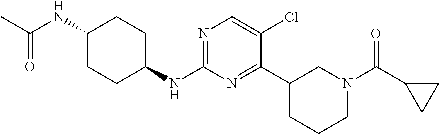 [SMILES: CC(=O)N[C@H]1CC[C@H](Nc2ncc(Cl)c(C3CCCN(C(=O)C4CC4)C3)n2)CC1] | trans-N-(4-((5-chloro-4-(1- (cyclopropanecarbonyl) piperidin-3-yl)pyrimidin- 2-yl)amino) cyclohexyl)acetamide |
| 101 | 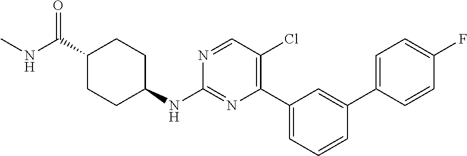 [SMILES: CNC(=O)[C@H]1CC[C@H](Nc2ncc(Cl)c(-c3cccc(-c4ccc(F)cc4)c3)n2)CC1] | trans-(1r,4r)-4-((5-chloro- 4-(4′-fluoro-[1,1′- biphenyl]-3-yl)pyrimidin- 2-yl)amino)-N- methylcyclohexane-1- carboxamide |
| 102 | 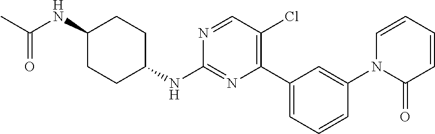 [SMILES: CC(=O)N[C@H]1CC[C@H](Nc2ncc(Cl)c(-c3cccc(-n4ccccc4=O)c3)n2)CC1] | trans-N-((1r,4r)-4-((5- chloro-4-(3-(2-oxopyridin- 1(2H)- yl)phenyl)pyrimidin-2- yl)amino)cyclohexyl) acetamide |
| 103 | 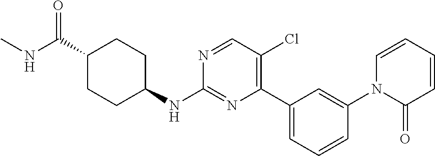 [SMILES: CNC(=O)[C@H]1CC[C@H](Nc2ncc(Cl)c(-c3cccc(-n4ccccc4=O)c3)n2)CC1] | trans-(1r,4r)-4-((5-chloro- 4-(3-(2-oxopyridin-1(2H)- yl)phenyl)pyrimidin-2- yl)amino)-N- methylcyclohexane-1- carboxamide |
| 104 | 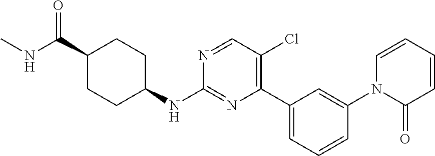 [SMILES: CNC(=O)[C@H]1CC[C@@H](Nc2ncc(Cl)c(-c3cccc(-n4ccccc4=O)c3)n2)CC1] | cis-(1s,4s)-4-((5-chloro-4- (3-(2-oxopyridin-1(2H)- yl)phenyl)pyrimidin-2- yl)amino)-N- methylcyclohexane-1- carboxamide |
| 105 | 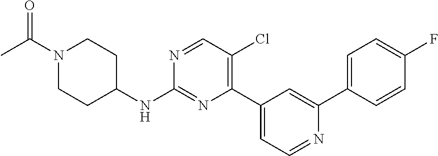 [SMILES: CC(=O)N1CCC(Nc2ncc(Cl)c(-c3ccnc(-c4ccc(F)cc4)c3)n2)CC1] | 1-(4-((5-chloro-4-(2-(4- fluorophenyl)pyridin-4- yl)pyrimidin-2- yl)amino)piperidin-1- yl)ethan-1-one |
| 106 | 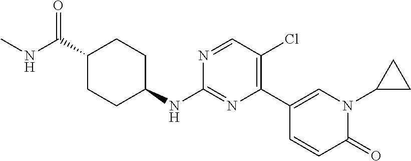 [SMILES: CNC(=O)[C@H]1CC[C@H](Nc2ncc(Cl)c(-c3ccc(=O)n(C4CC4)c3)n2)CC1] | trans-(1r,4r)-4-((5-chloro- 4-(1-cyclopropyl-6-oxo- 1,6-dihydropyridin-3- yl)pyrimidin-2-yl)amino)- N-methylcyclohexane-1- carboxamide |
| 107 | 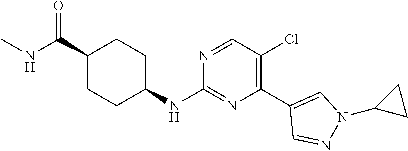 [SMILES: CNC(=O)[C@H]1CC[C@@H](Nc2ncc(Cl)c(-c3cnn(C4CC4)c3)n2)CC1] | cis-(1s,4s)-4-((5-chloro-4- (1-cyclopropyl-1H- pyrazol-4-yl)pyrimidin-2- yl)amino)-N- methylcyclohexane-1- carboxamide |
| 108 | 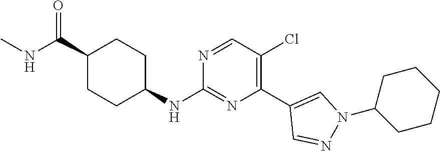 [SMILES: CNC(=O)[C@H]1CC[C@@H](Nc2ncc(Cl)c(-c3cnn(C4CCCCC4)c3)n2)CC1] | cis-(1s,4s)-4-((5-chloro-4- (1-cyclohexyl-1H-pyrazol- 4-yl)pyrimidin-2- yl)amino)-N- methylcyclohexane-1- carboxamide |
| 109 | 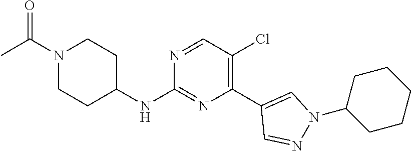 [SMILES: CC(=O)N1CCC(Nc2ncc(Cl)c(-c3cnn(C4CCCCC4)c3)n2)CC1] | 1-(4-((5-chloro-4-(1- cyclohexyl-1H-pyrazol-3- yl)pyrimidin-2- yl)amino)piperidin-1- yl)ethan-1-one |
| 110 | 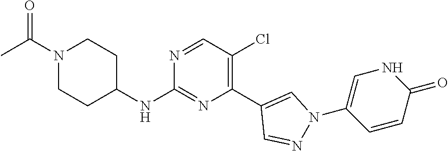 [SMILES: CC(=O)N1CCC(Nc2ncc(Cl)c(-c3cnn(-c4ccc(=O)[nH]c4)c3)n2)CC1] | 5-(4-(2-((1-acetylpiperidin- 4-yl)amino)-5- chloropyrimidin-4-yl)-1H- pyrazol-1-yl)pyridin- 2(1H)-one |
| 111 | 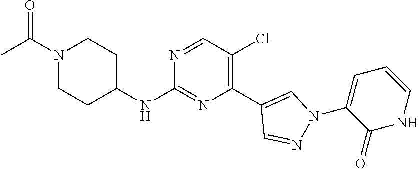 [SMILES: CC(=O)N1CCC(Nc2ncc(Cl)c(-c3cnn(-c4ccc[nH]c4=O)c3)n2)CC1] | 3-(4-(2-((1-acetylpiperidin- 4-yl)amino)-5- chloropyrimidin-4-yl)-1H- pyrazol-1-yl)pyridin- 2(1H)-one |
| 112 | 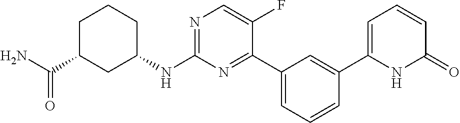 [SMILES: NC(=O)[C@@H]1CCC[C@H](Nc2ncc(F)c(-c3cccc(-c4cccc(=O)[nH]4)c3)n2)C1] | cis-3-((5-fluoro-4-(3-(6- oxo-1,6-dihydropyridin-2- yl)phenyl)pyrimidin-2- yl)amino)cyclohexane-1- carboxamide |
| 113 | 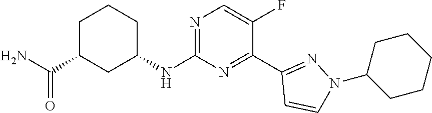 [SMILES: NC(=O)[C@@H]1CCC[C@H](Nc2ncc(F)c(-c3ccn(C4CCCCC4)n3)n2)C1] | cis-3-((4-(1-cyclohexyl- 1H-pyrazol-3-yl)-5- fluoropyrimidin-2- yl)amino)cyclohexane-1- carboxamide |
| 114 | 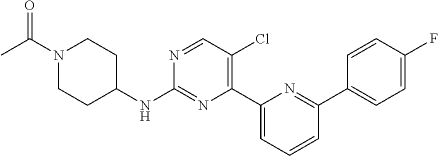 [SMILES: CC(=O)N1CCC(Nc2ncc(Cl)c(-c3cccc(-c4ccc(F)cc4)n3)n2)CC1] | 1-(4-((5-chloro-4-(6-(4- fluorophenyl)pyridin-2- yl)pyrimidin-2- yl)amino)piperidin-1- yl)ethan-1-one |
| 115 | 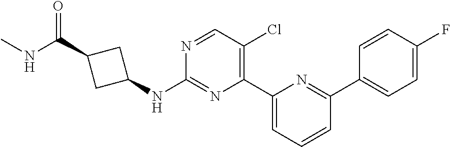 [SMILES: CNC(=O)[C@H]1C[C@@H](Nc2ncc(Cl)c(-c3cccc(-c4ccc(F)cc4)n3)n2)C1] | cis-3-((5-chloro-4-(6-(4- fluorophenyl)pyridin-2- yl)pyrimidin-2-yl)amino)- N-methylcyclobutane-1- carboxamide |
| 116 | 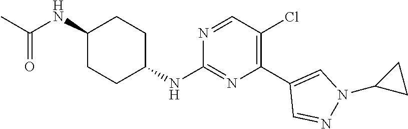 [SMILES: CC(=O)N[C@H]1CC[C@H](Nc2ncc(Cl)c(-c3cnn(C4CC4)c3)n2)CC1] | trans-N-((1r,4r)-4-((5- chloro-4-(1-cyclopropyl- 1H-pyraazol-4- yl)pyrimidin-2- yl)amino)cyclohexyl) acetamide |
| 117 | 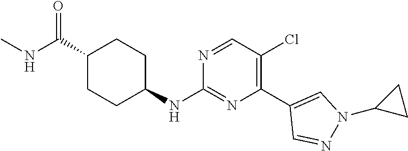 [SMILES: CNC(=O)[C@H]1CC[C@H](Nc2ncc(Cl)c(-c3cnn(C4CC4)c3)n2)CC1] | trans-(1r,4r)-4-((5-chloro- 4-(1-cyclopropyl-1H- pyrazol-4-yl)pyrimidin-2- yl)amino)-N- methylcyclohexane-1- carboxamide |
| 118 | 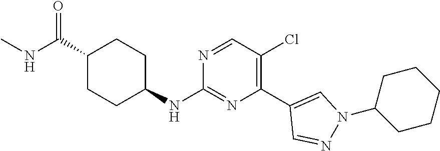 [SMILES: CNC(=O)[C@H]1CC[C@H](Nc2ncc(Cl)c(-c3cnn(C4CCCCC4)c3)n2)CC1] | trans-(1r,4r)-4-((5-chloro- 4-(1-cyclohexyl-1H- pyrazol-4-yl)pyrimidin-2- yl)amino)-N- methylcyclohexane-1- carboxamide |
| 119 | 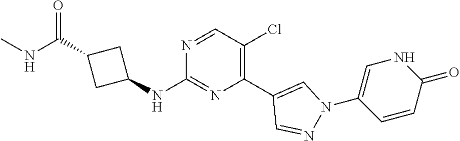 [SMILES: CNC(=O)[C@H]1C[C@H](Nc2ncc(Cl)c(-c3cnn(-c4ccc(=O)[nH]c4)c3)n2)C1] | trans-(1r,3r)-3-((5-chloro- 4-(1-(6-oxo-1,6- dihydropyridin-3-yl)-1H- pyrazol-4-yl)pyrimidin-2- yl)amino)-N- methylcyclobutane-1- carboxamide |
| 120 | 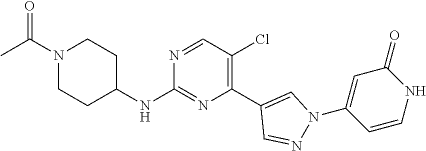 [SMILES: CC(=O)N1CCC(Nc2ncc(Cl)c(-c3cnn(-c4cc[nH]c(=O)c4)c3)n2)CC1] | 4-(4-(2-((1-acetylpiperidin- 4-yl)amino)-5- chloropyrimidin-4-yl)-1H- pyrazol-1-yl)pyridin- 2(1H)-one |
| 121 | 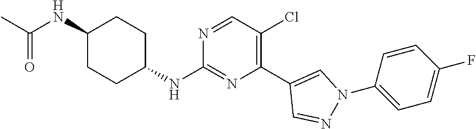 [SMILES: CC(=O)N[C@H]1CC[C@H](Nc2ncc(Cl)c(-c3cnn(-c4ccc(F)cc4)c3)n2)CC1] | trans-N-((1r,4r)-4-((5- chloro-4-(1-(4- fluorophenyl)-1H-pyrazol- 4-yl)pyrimidin-2- yl)amino)cyclohexyl) acetamide |
| 122 | 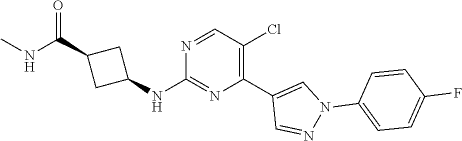 [SMILES: CNC(=O)[C@H]1C[C@@H](Nc2ncc(Cl)c(-c3cnn(-c4ccc(F)cc4)c3)n2)C1] | cis-3-((5-chloro-4-(1-(4- fluorophenyl)-1H-pyrazol- 4-yl)pyrimidin-2- yl)amino)-N- methylcyclobutane-1- carboxamide |
| 123 | 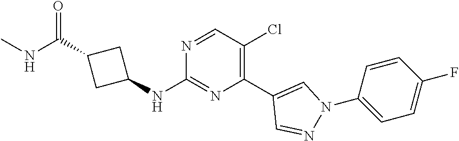 [SMILES: CNC(=O)[C@H]1C[C@H](Nc2ncc(Cl)c(-c3cnn(-c4ccc(F)cc4)c3)n2)C1] | trans-(1r,3r)-3-((5-chloro- 4-(1-(4-fluorophenyl)-1H- pyrazol-4-yl)pyrimidin-2- yl)amino)-N- methylcyclobutane-1- carboxamide |
| 124 | 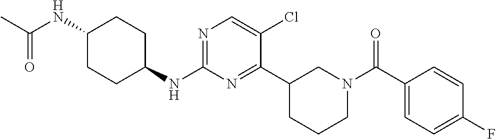 [SMILES: CC(=O)N[C@H]1CC[C@H](Nc2ncc(Cl)c(C3CCCN(C(=O)c4ccc(F)cc4)C3)n2)CC1] | trans-N-(4-((5-chloro-4-(1- (4-fluorobenzoyl)piperidin- 3-yl)pyrimidin-2- yl)amino)cyclohexyl) acetamide |
| 125 | 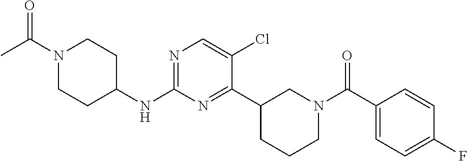 [SMILES: CC(=O)N1CCC(Nc2ncc(Cl)c(C3CCCN(C(=O)c4ccc(F)cc4)C3)n2)CC1] | 1-(4-((5-chloro-4-(1-(4- fluorobenzoyl)piperidin-3- yl)pyrimidin-2- yl)amino)piperidin-1- yl)ethan-1-one |
| 126 | 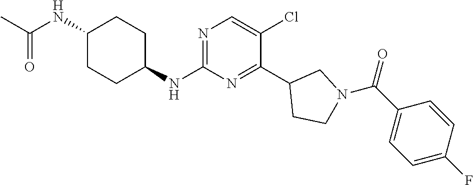 [SMILES: CC(=O)N[C@H]1CC[C@H](Nc2ncc(Cl)c(C3CCN(C(=O)c4ccc(F)cc4)C3)n2)CC1] | trans-N-(4-((5-chloro-4-(1- (4- fluorobenzoyl)pyrrolidin- 3-yl)pyrimidin-2- yl)amino)cyclohexyl) acetamide |
| 127 | 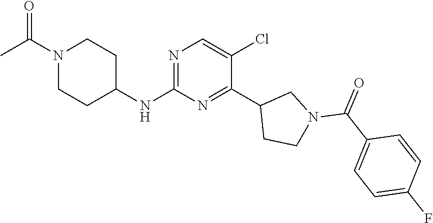 [SMILES: CC(=O)N1CCC(Nc2ncc(Cl)c(C3CCN(C(=O)c4ccc(F)cc4)C3)n2)CC1] | 1-(4-((5-chloro-4-(1-(4- fluorobenzoyl)pyrrolidin- 3-yl)pyrimidin-2- yl)amino)piperidin-1- yl)ethan-1-one |
| 128 | 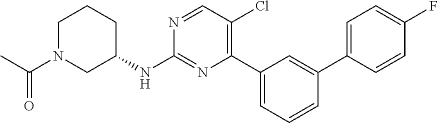 [SMILES: CC(=O)N1CCC[C@H](Nc2ncc(Cl)c(-c3cccc(-c4ccc(F)cc4)c3)n2)C1] | (S)-1-(3-((5-chloro-4-(4′- fluoro-[1,1′-biphenyl]-3- yl)pyrimidin-2- yl)amino)piperidin-1- yl)ethan-1-one |
| 129 | 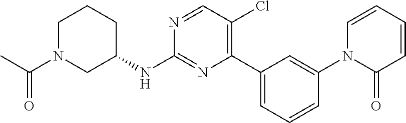 [SMILES: CC(=O)N1CCC[C@H](Nc2ncc(Cl)c(-c3cccc(-n4ccccc4=O)c3)n2)C1] | (S)-1-(3-(2-((1- acetylpiperidin-3- yl)amino)-5- chloropyrimidin-4- yl)phenyl)pyridin-2(1H)- one |
| 130 | 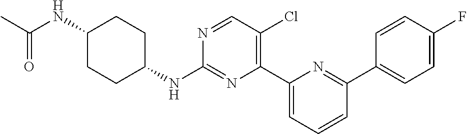 [SMILES: CC(=O)N[C@H]1CC[C@@H](Nc2ncc(Cl)c(-c3cccc(-c4ccc(F)cc4)n3)n2)CC1] | cis-N-((1s,4s)-4-((5-chloro- 4-(6-(4- fluorophenyl)pyridin-2- yl)pyrimidin-2- yl)amino)cyclohexyl) acetamide |
| 131 | 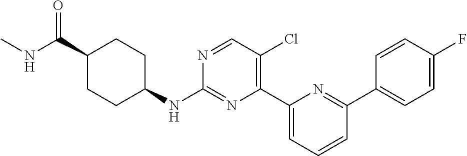 [SMILES: CNC(=O)[C@H]1CC[C@@H](Nc2ncc(Cl)c(-c3cccc(-c4ccc(F)cc4)n3)n2)CC1] | cis-(1s,4s)-4-((5-chloro-4- (6-(4-fluorophenyl)pyridin- 2-yl)pyrimidin-2- yl)amino)-N- methylcyclohexane-1- carboxamide |
| 132 | 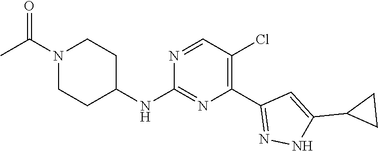 [SMILES: CC(=O)N1CCC(Nc2ncc(Cl)c(-c3cc(C4CC4)[nH]n3)n2)CC1] | 1-(4-((5-chloro-4-(5- cyclopropyl-1H-pyrazol-3- yl)pyrimidin-2- yl)amino)piperidin-1- yl)ethan-1-one |
| 133 | 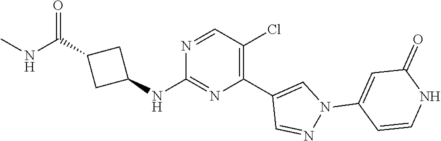 [SMILES: CNC(=O)[C@H]1C[C@H](Nc2ncc(Cl)c(-c3cnn(-c4cc[nH]c(=O)c4)c3)n2)C1] | trans-(1r,3r)-3-((5-chloro- 4-(1-(2-oxo-1,2- dihydropyridin-4-yl)-1H- pyrazol-4-yl)pyrimidin-2- yl)amino)-N- methylcyclobutane-1- carboxamide |
| 134 | 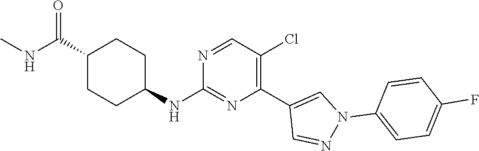 [SMILES: CNC(=O)[C@H]1CC[C@H](Nc2ncc(Cl)c(-c3cnn(-c4ccc(F)cc4)c3)n2)CC1] | trans-(1r,4r)-4-((5-chloro- 4-(1-(4-fluorophenyl)-1H- pyrazol-4-yl)pyrimidin-2- yl)amino)-N- methylcyclohexane-1- carboxamide |
| 135 | 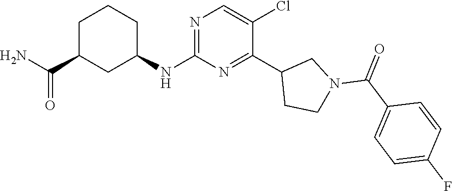 [SMILES: NC(=O)[C@H]1CCC[C@@H](Nc2ncc(Cl)c(C3CCN(C(=O)c4ccc(F)cc4)C3)n2)C1] | cis-3-((5-chloro-4-(1-(4- fluorobenzoyl)pyrrolidin- 3-yl)pyrimidin-2- yl)amino)cyclohexane-1- carboxamide |
| 136 | 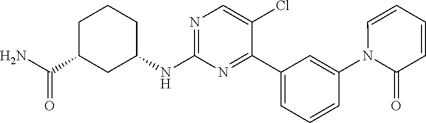 [SMILES: NC(=O)[C@@H]1CCC[C@H](Nc2ncc(Cl)c(-c3cccc(-n4ccccc4=O)c3)n2)C1] | cis-3-((5-chloro-4-(3-(2- oxopyridin-1(2H)- yl)phenyl)pyrimidin-2- yl)amino)cyclohexane-1- carboxamide |
| 137 | 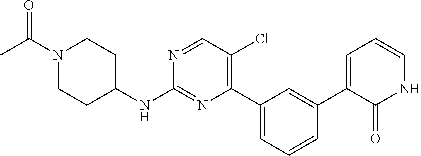 [SMILES: CC(=O)N1CCC(Nc2ncc(Cl)c(-c3cccc(-c4ccc[nH]c4=O)c3)n2)CC1] | 3-(3-(2-((1-acetylpiperidin- 4-yl)amino)-5- chloropyrimidin-4- yl)phenyl)pyridin-2(1H)- one |
| 138 | 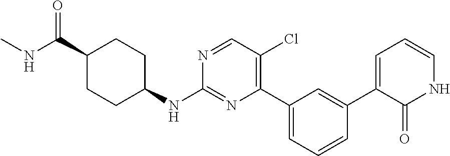 [SMILES: CNC(=O)[C@H]1CC[C@@H](Nc2ncc(Cl)c(-c3cccc(-c4ccc[nH]c4=O)c3)n2)CC1] | cis-(1s,4s)-4-((5-chloro-4- (3-(2-oxo-1,2- dihydropyridin-3- yl)phenyl)pyrimidin-2- yl)amino)-N- methylcyclohexane-1- carboxamide |
| 139 | 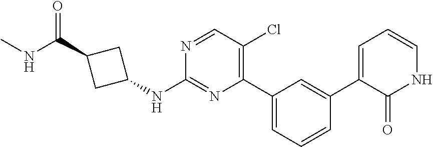 [SMILES: CNC(=O)[C@H]1C[C@H](Nc2ncc(Cl)c(-c3cccc(-c4ccc[nH]c4=O)c3)n2)C1] | trans-(1r,3r)-3-((5-chloro- 4-(3-(2-oxo-1,2- dihydropyridin-3- yl)phenyl)pyrimidin-2- yl)amino)-N- methylcyclobutane-1- carboxamide |
| 140 | 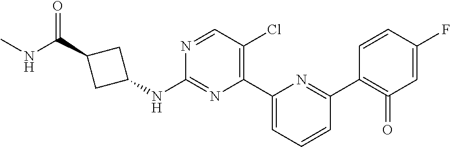 | trans-(1r,3r)-3-((5-chloro- 4-(6-(4- fluorophenyl)pyridin-2- yl)pyrimidin-2-yl)amino)- N-methylcyclobutane-1- carboxamide |
| 141 | 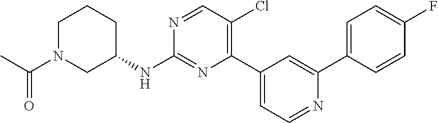 [SMILES: CC(=O)N1CCC[C@H](Nc2ncc(Cl)c(-c3ccnc(-c4ccc(F)cc4)c3)n2)C1] | (S)-1-(3-((5-chloro-4-(2- (4-fluorophenyl)pyridin-4- yl)pyrimidin-2- yl)amino)piperidin-1- yl)ethan-1-one |
| 142 | 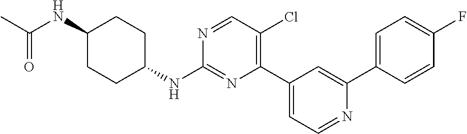 [SMILES: CC(=O)N[C@H]1CC[C@H](Nc2ncc(Cl)c(-c3ccnc(-c4ccc(F)cc4)c3)n2)CC1] | trans-N-((1r,4r)-4-((5- chloro-4-(2-(4- fluorophenyl)pyridin-4- yl)pyrimidin-2- yl)amino)cyclohexyl) acetamide |
| 143 | 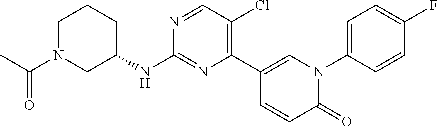 [SMILES: CC(=O)N1CCC[C@H](Nc2ncc(Cl)c(-c3ccc(=O)n(-c4ccc(F)cc4)c3)n2)C1] | (S)-5-(2-((1- acetylpiperidin-3- yl)amino)-5- chloropyrimidin-4-yl)-1- (4-fluorophenyl)pyridin- 2(1H)-one |
| 144 | 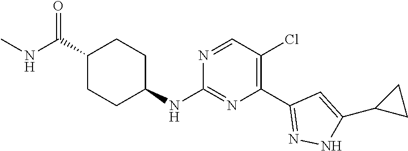 [SMILES: CNC(=O)[C@H]1CC[C@H](Nc2ncc(Cl)c(-c3cc(C4CC4)[nH]n3)n2)CC1] | trans-(1r,4r)-4-((5-chloro- 4-(5-cyclopropyl-1H- pyrazol-3-yl)pyrimidin-2- yl)amino)-N- methylcyclohexane-1- carboxamide |
| 145 | 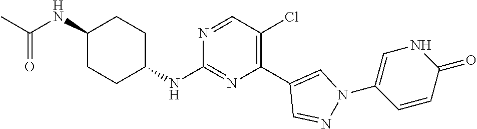 [SMILES: CC(=O)N[C@H]1CC[C@H](Nc2ncc(Cl)c(-c3cnn(-c4ccc(=O)[nH]c4)c3)n2)CC1] | trans-N-((1r,4r)-4-((5- chloro-4-(1-(6-oxo-1,6- dihydropyridin-3-yl)-1H- pyrazol-4-yl)pyrimidin-2- yl)amino)cyclohexyl) acetamide |
| 146 | 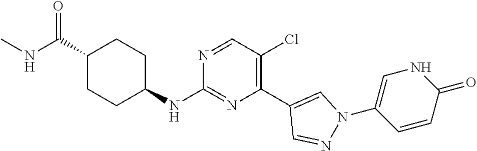 [SMILES: CNC(=O)[C@H]1CC[C@H](Nc2ncc(Cl)c(-c3cnn(-c4ccc(=O)[nH]c4)c3)n2)CC1] | trans-(1r,4r)-4-((5-chloro- 4-(1-(6-oxo-1,6- dihydropyridin-3-yl)-1H- pyrazol-4-yl)pyrimidin-2- yl)amino)-N- methylcyclohexane-1- carboxamide |
| 147 | 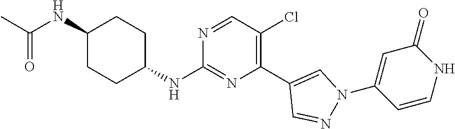 [SMILES: CC(=O)N[C@H]1CC[C@H](Nc2ncc(Cl)c(-c3cnn(-c4cc[nH]c(=O)c4)c3)n2)CC1] | trans-N-((1r,4r)-4-((5- chloro-4-(1-(2-oxo-1,2- dihydropyridin-4-yl)-1H- pyrazol-4-yl)pyrimidin-2- yl)amino)cyclohexyl) acetamide |
| 148 | 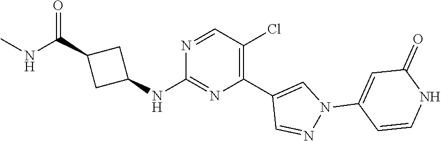 [SMILES: CNC(=O)[C@H]1C[C@@H](Nc2ncc(Cl)c(-c3cnn(-c4cc[nH]c(=O)c4)c3)n2)C1] | cis-(1s,4s)-4-((5-chloro-4- (1-(2-oxo-1,2- dihydropyridin-4-yl)-1H- pyrazol-4-yl)pyrimidin-2- yl)amino)-N- methylcyclohexane-1- carboxamide |
| 149 | 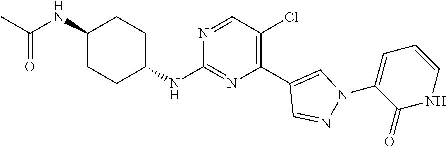 [SMILES: CC(=O)N[C@H]1CC[C@H](Nc2ncc(Cl)c(-c3cnn(-c4ccc[nH]c4=O)c3)n2)CC1] | trans-N-((1r,4r)-4-((5- chloro-4-(1-(2-oxo-1,2- dihydropyridin-3-yl)-1H- pyrazol-4-yl)pyrimidin-2- yl)amino)cyclohexyl) acetamide |
| 150 | 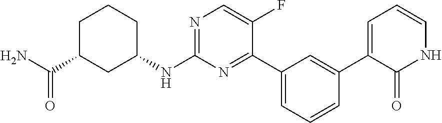 [SMILES: NC(=O)[C@@H]1CCC[C@H](Nc2ncc(F)c(-c3cccc(-c4ccc[nH]c4=O)c3)n2)C1] | cis-3-((5-fluoro-4-(3-(2- oxo-1,2-dihydropyridin-3- yl)phenyl)pyrimidin-2- yl)amino)cyclohexane-1- carboxamide |
| 152 | 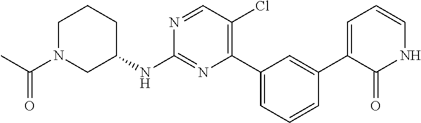 [SMILES: CC(=O)N1CCC[C@H](Nc2ncc(Cl)c(-c3cccc(-c4ccc[nH]c4=O)c3)n2)C1] | (S)-3-(3-(2-((1- acetylpiperidin-3- yl)amino)-5- chloropyrimidin-4- yl)phenyl)pyridin-2(1H)- one |
| 153 | 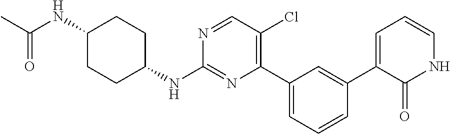 [SMILES: CC(=O)N[C@H]1CC[C@@H](Nc2ncc(Cl)c(-c3cccc(-c4ccc[nH]c4=O)c3)n2)CC1] | cis-N-((1s,4s)-4-((5-chloro- 4-(3-(2-oxo-1,2- dihydropyridin-3- yl)phenyl)pyrimidin-2- yl)amino)cyclohexyl) acetamide |
| 154 | 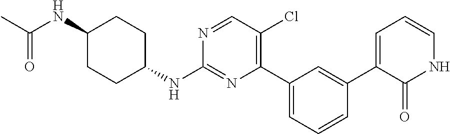 [SMILES: CC(=O)N[C@H]1CC[C@H](Nc2ncc(Cl)c(-c3cccc(-c4ccc[nH]c4=O)c3)n2)CC1] | trans-N-((1r,4r)-4-((5- chloro-4-(3-(2-oxo-1,2- dihydropyridin-3- yl)phenyl)pyrimidin-2- yl)amino)cyclohexyl) acetamide |
| 155 | 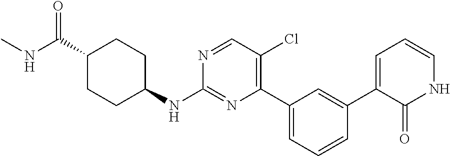 [SMILES: CNC(=O)[C@H]1CC[C@H](Nc2ncc(Cl)c(-c3cccc(-c4ccc[nH]c4=O)c3)n2)CC1] | trans-(1r,4r)-4-((5-chloro- 4-(3-(2-oxo-1,2- dihydropyridin-3- yl)phenyl)pyrimidin-2- yl)amino)-N- methylcyclohexane-1- carboxamide |
| 156 | 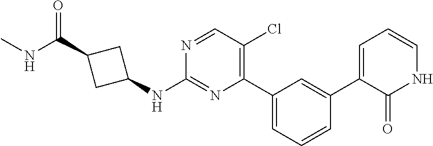 [SMILES: CNC(=O)[C@H]1C[C@@H](Nc2ncc(Cl)c(-c3cccc(-c4ccc[nH]c4=O)c3)n2)C1] | cis-3-((5-chloro-4-(3-(2- oxo-1,2-dihydropyridin-3- yl)phenyl)pyrimidin-2- yl)amino)-N- methylcyclobutane-1- carboxamide |
| 157 | 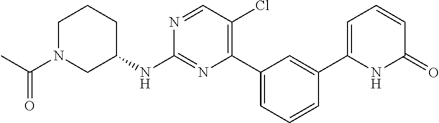 [SMILES: CC(=O)N1CCC[C@H](Nc2ncc(Cl)c(-c3cccc(-c4cccc(=O)[nH]4)c3)n2)C1] | (S)-6-(3-(2-((1- acetylpiperidin-3- yl)amino)-5- chloropyrimidin-4- yl)phenyl)pyridin-2(1H)- one |
| 158 | 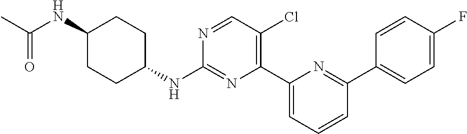 [SMILES: CC(=O)N[C@H]1CC[C@H](Nc2ncc(Cl)c(-c3cccc(-c4ccc(F)cc4)n3)n2)CC1] | trans-N-((1r,4r)-4-((5- chloro-4-(6-(4- fluorophenyl)pyridin-2- yl)pyrimidin-2- yl)amino)cyclohexyl) acetamide |
| 159 | 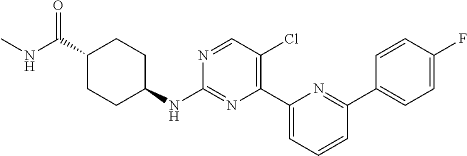 [SMILES: CNC(=O)[C@H]1CC[C@H](Nc2ncc(Cl)c(-c3cccc(-c4ccc(F)cc4)n3)n2)CC1] | trans-(1r,4r)-4-((5-chloro- 4-(6-(4- fluorophenyl)pyridin-2- yl)pyrimidin-2-yl)amino)- N-methylcyclohexane-1- carboxamide |
| 160 | 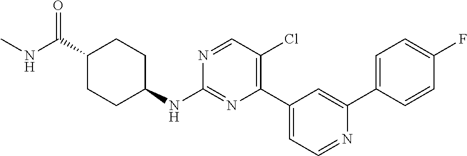 [SMILES: CNC(=O)[C@H]1CC[C@H](Nc2ncc(Cl)c(-c3ccnc(-c4ccc(F)cc4)c3)n2)CC1] | trans-(1r,4r)-4-((5-chloro- 4-(2-(4- fluorophenyl)pyridin-4- yl)pyrimidin-2-yl)amino)- N-methylcyclohexane-1- carboxamide |
| 161 | 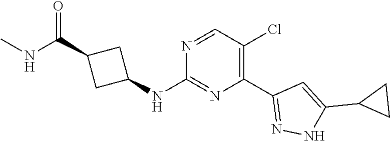 [SMILES: CNC(=O)[C@H]1C[C@@H](Nc2ncc(Cl)c(-c3cc(C4CC4)[nH]n3)n2)C1] | cis-3-((5-chloro-4-(5- cyclopropyl-1H-pyrazol-3- yl)pyrimidin-2-yl)amino)- N-methylcyclobutane-1- carboxamide |
| 162 | 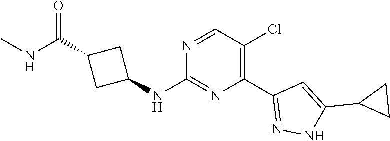 [SMILES: CNC(=O)[C@H]1C[C@H](Nc2ncc(Cl)c(-c3cc(C4CC4)[nH]n3)n2)C1] | trans-(1r,3r)-3-((5-chloro- 4-(5-cyclopropyl-1H- pyrazol-3-yl)pyrimidin-2- yl)amino)-N- methylcyclobutane-1- carboxamide |
| 163 | 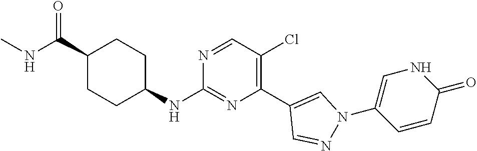 [SMILES: CNC(=O)[C@H]1CC[C@@H](Nc2ncc(Cl)c(-c3cnn(-c4ccc(=O)[nH]c4)c3)n2)CC1] | cis-(1s,4s)-4-((5-chloro-4- (1-(6-oxo-1,6- dihydropyridin-3-yl)-1H- pyrazol-4-yl)pyrimidin-2- yl)amino)-N- methylcyclohexane-1- carboxamide |
| 164 | 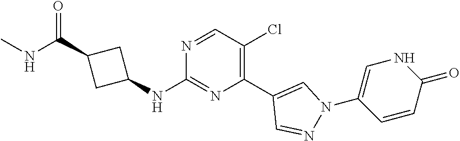 [SMILES: CNC(=O)[C@H]1C[C@@H](Nc2ncc(Cl)c(-c3cnn(-c4ccc(=O)[nH]c4)c3)n2)C1] | cis-3-((5-chloro-4-(1-(6- oxo-1,6-dihydropyridin-3- yl)-1H-pyrazol-4- yl)pyrimidin-2-yl)amino)- N-methylcyclobutane-1- carboxamide |
| 165 | 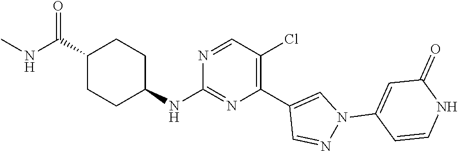 [SMILES: CNC(=O)[C@H]1CC[C@H](Nc2ncc(Cl)c(-c3cnn(-c4cc[nH]c(=O)c4)c3)n2)CC1] | trans-(1r,4r)-4-((5-chloro- 4-(1-(2-oxo-1,2- dihydropyridin-4-yl)-1H- pyrazol-4-yl)pyrimidin-2- yl)amino)-N- methylcyclohexane-1- carboxamide |
| 166 | 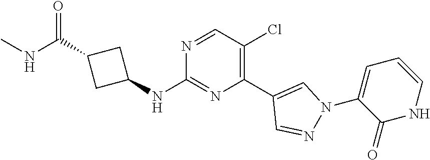 [SMILES: CNC(=O)[C@H]1C[C@H](Nc2ncc(Cl)c(-c3cnn(-c4ccc[nH]c4=O)c3)n2)C1] | trans-(1r,3r)-3-((5-chloro- 4-(1-(2-oxo-1,2- dihydropyridin-3-yl)-1H- pyrazol-4-yl)pyrimidin-2- yl)amino)-N- methylcyclobutane-1- carboxamide |
| 167 | 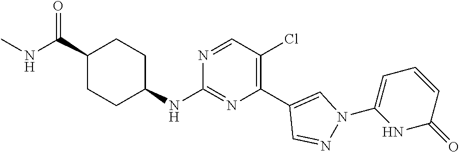 [SMILES: CNC(=O)[C@H]1CC[C@@H](Nc2ncc(Cl)c(-c3cnn(-c4cccc(=O)[nH]4)c3)n2)CC1] | cis-(1s,4s)-4-((5-chloro-4- (1-(6-oxo-1,6- dihydropyridin-2-yl)-1H- pyrazol-4-yl)pyrimidin-2- yl)amino)-N- methylcyclohexane-1- carboxamide |
| 168 | 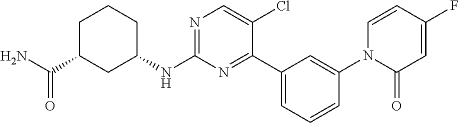 [SMILES: NC(=O)[C@@H]1CCC[C@H](Nc2ncc(Cl)c(-c3cccc(-n4ccc(F)cc4=O)c3)n2)C1] | cis-methyl 3-((5-chloro-4- (3-(4-fluoro-2-oxopyridin- 1(2H)- yl)phenyl)pyrimidin-2- yl)amino)cyclohexane-1- carboxylate |
| 169 | 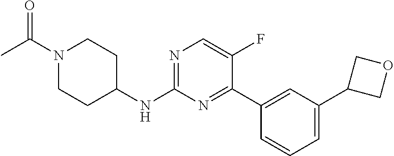 [SMILES: CC(=O)N1CCC(Nc2ncc(F)c(-c3cccc(C4COC4)c3)n2)CC1] | 1-(4-((5-fluoro-4-(3- (oxetan-3- yl)phenyl)pyrimidin-2- yl)amino)piperidin-1- yl)ethan-1-one |
| 170 | 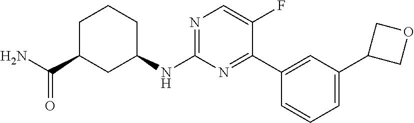 [SMILES: NC(=O)[C@H]1CCC[C@@H](Nc2ncc(F)c(-c3cccc(C4COC4)c3)n2)C1] | cis-3-((5-fluoro-4-(3- (oxetan-3- yl)phenyl)pyrimidin-2- yl)amino)cyclohexane-1- carboxamide |
| 171 | 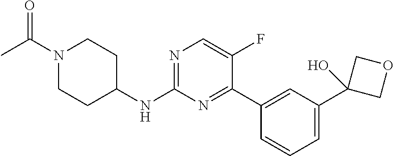 [SMILES: CC(=O)N1CCC(Nc2ncc(F)c(-c3cccc(C4(O)COC4)c3)n2)CC1] | 1-(4-((5-fluoro-4-(3-(3- hydroxyoxetan-3- yl)phenyl)pyrimidin-2- yl)amino)piperidin-1- yl)ethan-1-one |
| 172 | 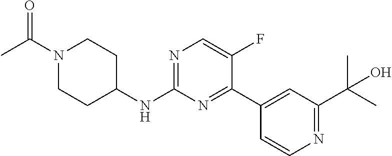 [SMILES: CC(=O)N1CCC(Nc2ncc(F)c(-c3ccnc(C(C)(C)O)c3)n2)CC1] | 1-(4-((5-fluoro-4-(2-(2- hydroxypropan-2- yl)pyridin-4-yl)pyrimidin- 2-yl)amino)piperidin-1- yl)ethan-1-one |
| 173 | 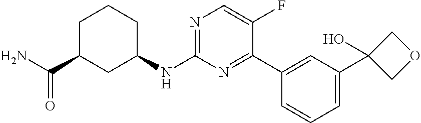 [SMILES: NC(=O)[C@H]1CCC[C@@H](Nc2ncc(F)c(-c3cccc(C4(O)COC4)c3)n2)C1] | cis-3-((5-fluoro-4-(3-(3- hydroxyoxetan-3- yl)phenyl)pyrimidin-2- yl)amino)cyclohexane-1- carboxamide |
| 174 | 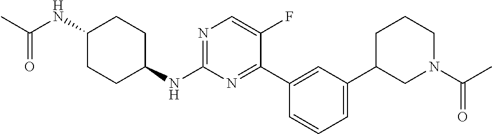 [SMILES: CC(=O)N[C@H]1CC[C@H](Nc2ncc(F)c(-c3cccc(C4CCCN(C(C)=O)C4)c3)n2)CC1] | trans-N-(4-((4-(3-(1- acetylpiperidin-3- yl)phenyl)-5- fluoropyrimidin-2- yl)amino)cyclohexyl) acetamide |
| 175 | 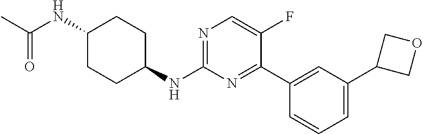 [SMILES: CC(=O)N[C@H]1CC[C@H](Nc2ncc(F)c(-c3cccc(C4COC4)c3)n2)CC1] | N-(trans-4-((5-fluoro-4-(3- (oxetan-3- yl)phenyl)pyrimidin-2- yl)amino)cyclohexyl) acetamide |
| 176 | 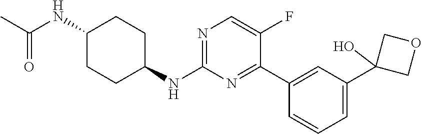 [SMILES: CC(=O)N[C@H]1CC[C@H](Nc2ncc(F)c(-c3cccc(C4(O)COC4)c3)n2)CC1] | N-(trans-4-((5-fluoro-4-(3- (3-hydroxyoxetan-3- yl)phenyl)pyrimidin-2- yl)amino)cyclohexyl) acetamide |
| 177 | 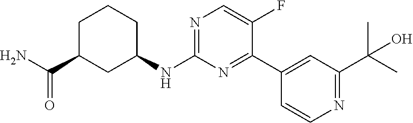 [SMILES: CC(C)(O)c1cc(-c2nc(N[C@@H]3CCC[C@H](C(N)=O)C3)ncc2F)ccn1] | cis-3-((5-fluoro-4-(2-(2- hydroxypropan-2- yl)pyridin-4-yl)pyrimidin- 2-yl)amino)cyclohexane-1- carboxamide |
| 178 | 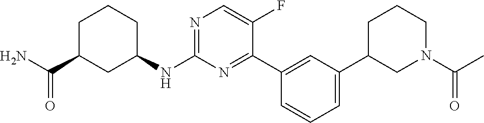 [SMILES: CC(=O)N1CCCC(c2cccc(-c3nc(N[C@@H]4CCC[C@H](C(N)=O)C4)ncc3F)c2)C1] | cis-3-((4-(3-(1- acetylpiperidin-3- yl)phenyl)-5- fluoropyrimidin-2- yl)amino)cyclohexane-1- carboxamide |
| 179 | 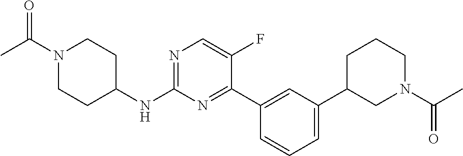 [SMILES: CC(=O)N1CCC(Nc2ncc(F)c(-c3cccc(C4CCCN(C(C)=O)C4)c3)n2)CC1] | 1-(4-((4-(3-(1- acetylpiperidin-3- yl)phenyl)-5- fluoropyrimidin-2- yl)amino)piperidin-1- yl)ethan-1-one |
| 180 | 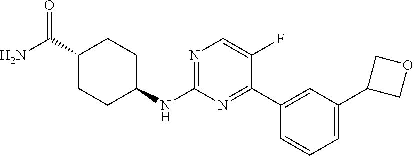 [SMILES: NC(=O)[C@H]1CC[C@H](Nc2ncc(F)c(-c3cccc(C4COC4)c3)n2)CC1] | trans-4-((5-fluoro-4-(3- (oxetan-3- yl)phenyl)pyrimidin-2- yl)amino)cyclohexane-1- carboxamide |
| 181 | 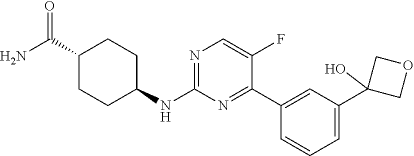 [SMILES: NC(=O)[C@H]1CC[C@H](Nc2ncc(F)c(-c3cccc(C4(O)COC4)c3)n2)CC1] | trans-4-((5-fluoro-4-(3-(3- hydroxyoxetan-3- yl)phenyl)pyrimidin-2- yl)amino)cyclohexane-1- carboxamide |
| 182 | 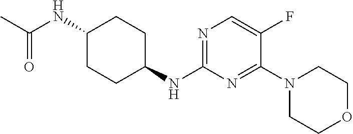 [SMILES: CC(=O)N[C@H]1CC[C@H](Nc2ncc(F)c(N3CCOCC3)n2)CC1] | trans-N-(4-((5-fluoro-4- morpholinopyrimidin-2- yl)amino)cyclohexyl) acetamide |
| 187 | 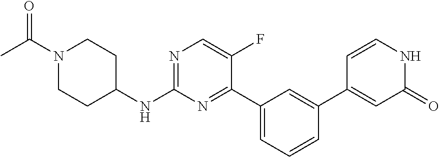 [SMILES: CC(=O)N1CCC(Nc2ncc(F)c(-c3cccc(-c4cc[nH]c(=O)c4)c3)n2)CC1] | 4-(3-(2-((1-acetylpiperidin- 4-yl)amino)-5- fluoropyrimidin-4- yl)phenyl)pyridin-2(1H)- one |
| 188 | 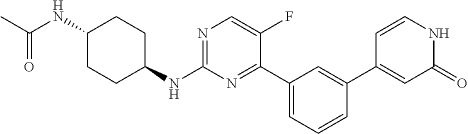 [SMILES: CC(=O)N[C@H]1CC[C@H](Nc2ncc(F)c(-c3cccc(-c4cc[nH]c(=O)c4)c3)n2)CC1] | trans-N-(4-((5-fluoro-4-(3- (2-oxo-1,2-dihydropyridin- 4-yl)phenyl)pyrimidin-2- yl)amino)cyclohexyl) acetamide |
| 189 | 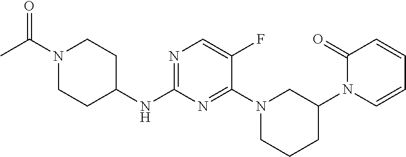 [SMILES: CC(=O)N1CCC(Nc2ncc(F)c(N3CCCC(n4ccccc4=O)C3)n2)CC1] | 1-(1-(2-((1-acetylpiperidin- 4-yl)amino)-5- fluoropyrimidin-4- yl)piperidin-3-yl)pyridin- 2(1H)-one |
| 190 | 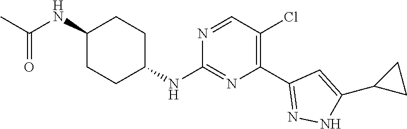 [SMILES: CC(=O)N[C@H]1CC[C@H](Nc2ncc(Cl)c(-c3cc(C4CC4)[nH]n3)n2)CC1] | trans-N-((1r,4r)-4-((5- chloro-4-(5-cyclopropyl- 1H-pyrazol-3- yl)pyrimidin-2- yl)amino)cyclohexyl) acetamide |
| 191 | 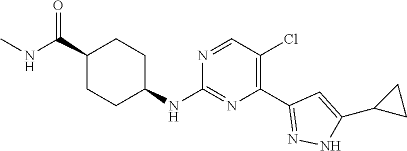 [SMILES: CNC(=O)[C@H]1CC[C@@H](Nc2ncc(Cl)c(-c3cc(C4CC4)[nH]n3)n2)CC1] | cis-(1s,4s)-4-((5-chloro-4- (5-cyclopropyl-1H- pyrazol-3-yl)pyrimidin-2- yl)amino)-N- methylcyclohexane-1- carboxamide |
| 192 | 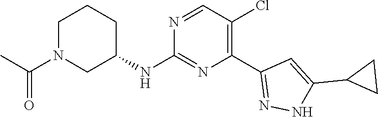 [SMILES: CC(=O)N1CCC[C@H](Nc2ncc(Cl)c(-c3cc(C4CC4)[nH]n3)n2)C1] | (S)-1-(3-((5-chloro-4-(5- cyclopropyl-1H-pyrazol-3- yl)pyrimidin-2- yl)amino)piperidin-1- yl)ethan-1-one |
| 193 | 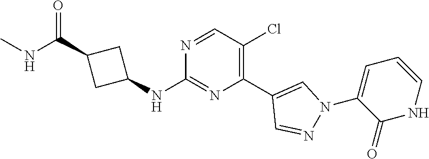 [SMILES: CNC(=O)[C@H]1C[C@@H](Nc2ncc(Cl)c(-c3cnn(-c4ccc[nH]c4=O)c3)n2)C1] | cis-3-((5-chloro-4-(1-(2- oxo-1,2-dihydropyridin-3- yl)-1H-pyrazol-4- yl)pyrimidin-2-yl)amino)- N-methylcyclobutane-1- carboxamide |
| 194 | 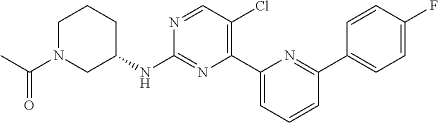 [SMILES: CC(=O)N1CCC[C@H](Nc2ncc(Cl)c(-c3cccc(-c4ccc(F)cc4)n3)n2)C1] | (S)-1-(3-((5-chloro-4-(6- (4-fluorophenyl)pyridin-2- yl)pyrimidin-2- yl)amino)piperidin-1- yl)ethan-1-one |
| 195 | 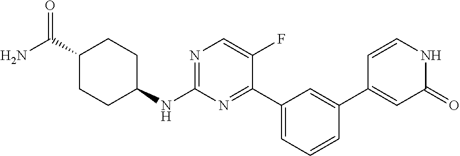 [SMILES: NC(=O)[C@H]1CC[C@H](Nc2ncc(F)c(-c3cccc(-c4cc[nH]c(=O)c4)c3)n2)CC1] | trans-4-((5-fluoro-4-(3-(2- oxo-1,2-dihydropyridin-4- yl)phenyl)pyrimidin-2- yl)amino)cyclohexane-1- carboxamide |
| 196 | 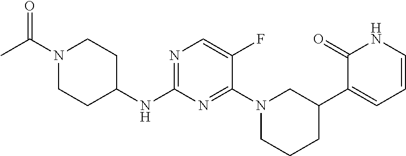 [SMILES: CC(=O)N1CCC(Nc2ncc(F)c(N3CCCC(c4ccc[nH]c4=O)C3)n2)CC1] | 3-[1-[2-[(1-acetyl-4- piperidyl)amino]-5-fluoro- pyrimidin-4-yl]-3- piperidyl]-1H-pyridin-2- one |
| 197 | 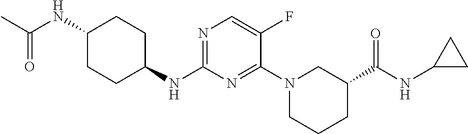 [SMILES: CC(=O)N[C@H]1CC[C@H](Nc2ncc(F)c(N3CCC[C@@H](C(=O)NC4CC4)C3)n2)CC1] | trans-(R)-1-(2-((4- acetamidocyclohexyl) amino)-5-fluoropyrimidin- 4-yl)-N-cyclopropyl- piperidine-3-carboxamide |
| 198 | 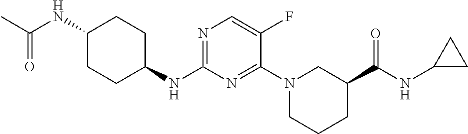 [SMILES: CC(=O)N[C@H]1CC[C@H](Nc2ncc(F)c(N3CCC[C@H](C(=O)NC4CC4)C3)n2)CC1] | trans-(S)-1-(2-((4- acetamidocyclohexyl) amino)-5-fluoropyrimidin- 4-yl)-N-cyclopropyl- piperidine-3-carboxamide |
| 199 | 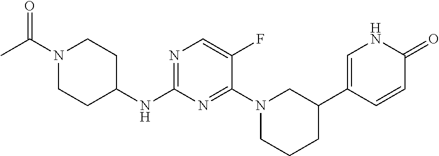 [SMILES: CC(=O)N1CCC(Nc2ncc(F)c(N3CCCC(c4ccc(=O)[nH]c4)C3)n2)CC1] | 5-(1-(2-((1-acetylpiperidin- 4-yl)amino)-5- fluoropyrimidin-4- yl)piperidin-3-yl)pyridin- 2(1H)-one |
| 200 | 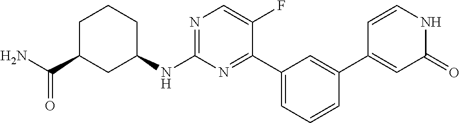 [SMILES: NC(=O)[C@H]1CCC[C@@H](Nc2ncc(F)c(-c3cccc(-c4cc[nH]c(=O)c4)c3)n2)C1] | cis-3-((5-fluoro-4-(3-(2- oxo-1,2-dihydropyridin-4- yl)phenyl)pyrimidin-2- yl)amino)cyclohexane-1- carboxamide |
| 201 | 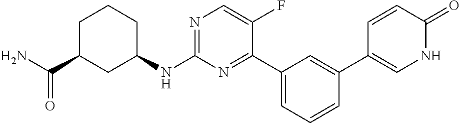 [SMILES: NC(=O)[C@H]1CCC[C@@H](Nc2ncc(F)c(-c3cccc(-c4ccc(=O)[nH]c4)c3)n2)C1] | rac-(1S,3R)-3-((5-fluoro-4- (3-(6-oxo-1,6- dihydropyridin-3- yl)phenyl)pyrimidin-2- yl)amino)cyclohexane-1- carboxamide |
| 202 | 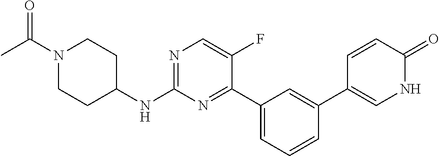 [SMILES: CC(=O)N1CCC(Nc2ncc(F)c(-c3cccc(-c4ccc(=O)[nH]c4)c3)n2)CC1] | 5-(3-(2-((1-acetylpiperidin- 4-yl)amino)-5- fluoropyrimidin-4- yl)phenyl)pyridin-2(1H)- one |
| 203 | 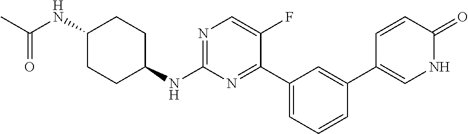 [SMILES: CC(=O)N[C@H]1CC[C@H](Nc2ncc(F)c(-c3cccc(-c4ccc(=O)[nH]c4)c3)n2)CC1] | trans-N-(4-((5-fluoro-4-(3- (6-oxo-1,6-dihydropyridin- 3-yl)phenyl)pyrimidin-2- yl)amino)cyclohexyl) acetamide |
| 204 | 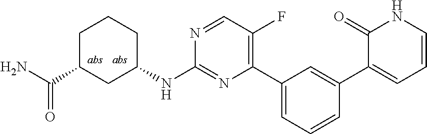 [SMILES: NC(=O)[C@@H]1CCC[C@H](Nc2ncc(F)c(-c3cccc(-c4ccc[nH]c4=O)c3)n2)C1] | (1R,3S)-3-((5-fluoro-4-(3- (2-oxo-1,2-dihydropyridin- 3-yl)phenyl)pyrimidin-2- yl)amino)cyclohexane-1- carboxamide |
| 205 | 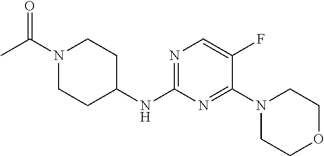 [SMILES: CC(=O)N1CCC(Nc2ncc(F)c(N3CCOCC3)n2)CC1] | 1-(4-((5-fluoro-4- morpholinopyrimidin-2- yl)amino)piperidin-1- yl)ethan-1-one |
| 206 | 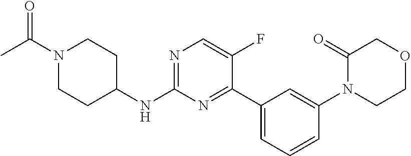 [SMILES: CC(=O)N1CCC(Nc2ncc(F)c(-c3cccc(N4CCOCC4=O)c3)n2)CC1] | 4-(3-(2-((1-acetylpiperidin- 4-yl)amino)-5- fluoropyrimidin-4- yl)phenyl)morpholin-3-one |
| 207 | 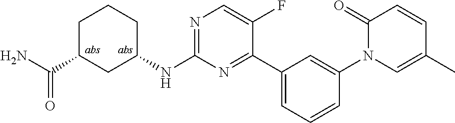 [SMILES: Cc1ccc(=O)n(-c2cccc(-c3nc(N[C@H]4CCC[C@@H](C(N)=O)C4)ncc3F)c2)c1] | (1R,3S)-3-((5-fluoro-4-(3- (5-methyl-2-oxopyridin- 1(2H)- yl)phenyl)pyrimidin-2- yl)amino)cyclohexane-1- carboxamide |
| 208 | 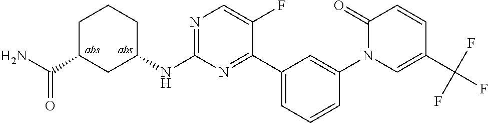 [SMILES: NC(=O)[C@@H]1CCC[C@H](Nc2ncc(F)c(-c3cccc(-n4cc(C(F)(F)F)ccc4=O)c3)n2)C1] | (1R,3S)-3-((5-fluoro-4-(3- (2-oxo-5- (trifluoromethyl)pyridin- 1(2H)- yl)phenyl)pyrimidin-2- yl)amino)cyclohexane-1- carboxamide |
| 209 | 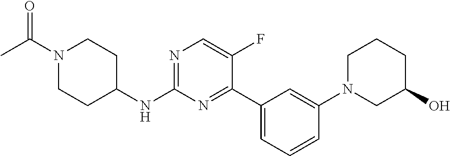 [SMILES: CC(=O)N1CCC(Nc2ncc(F)c(-c3cccc(N4CCC[C@@H](O)C4)c3)n2)CC1] | (R)-1-(4-((5-fluoro-4-(3- (3-hydroxypiperidin-1- yl)phenyl)pyrimidin-2- yl)amino)piperidin-1- yl)ethan-1-one |
| 210 | 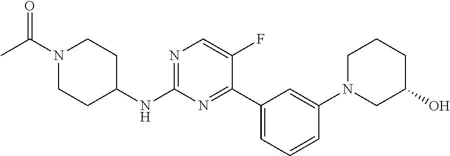 [SMILES: CC(=O)N1CCC(Nc2ncc(F)c(-c3cccc(N4CCC[C@H](O)C4)c3)n2)CC1] | (S)-1-(4-((5-fluoro-4-(3-(3- hydroxypiperidin-1- yl)phenyl)pyrimidin-2- yl)amino)piperidin-1- yl)ethan-1-one |
| 211 | 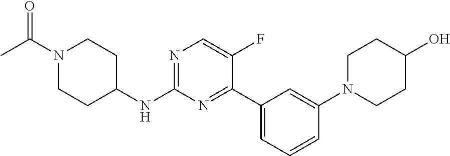 [SMILES: CC(=O)N1CCC(Nc2ncc(F)c(-c3cccc(N4CCC(O)CC4)c3)n2)CC1] | 1-(4-((5-fluoro-4-(3-(4- hydroxypiperidin-1- yl)phenyl)pyrimidin-2- yl)amino)piperidin-1- yl)ethan-1-one |
| 212 | 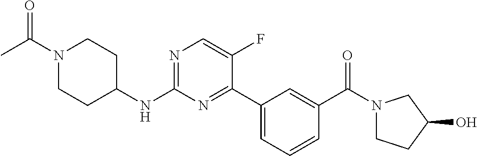 [SMILES: CC(=O)N1CCC(Nc2ncc(F)c(-c3cccc(C(=O)N4CC[C@H](O)C4)c3)n2)CC1] | (S)-1-(4-((5-fluoro-4-(3-(3- hydroxypyrrolidine-1- carbonyl)phenyl)pyrimidin- 2-yl)amino)piperidin-1- yl)ethan-1-one |
| 213 | 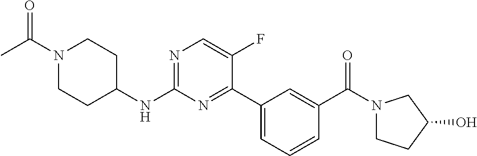 [SMILES: CC(=O)N1CCC(Nc2ncc(F)c(-c3cccc(C(=O)N4CC[C@@H](O)C4)c3)n2)CC1] | (R)-1-(4-((5-fluoro-4-(3- (3-hydroxypyrrolidine-1- carbonyl)phenyl)pyrimidin- 2-yl)amino)piperidin-1- yl)ethan-1-one |
| 214 | 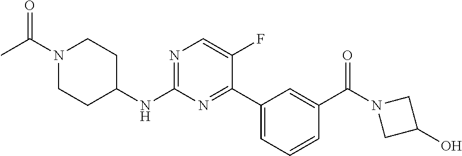 [SMILES: CC(=O)N1CCC(Nc2ncc(F)c(-c3cccc(C(=O)N4CC(O)C4)c3)n2)CC1] | 1-(4-((5-fluoro-4-(3-(3- hydroxyazetidine-1- carbonyl)phenyl)pyrimidin- 2-yl)amino)piperidin-1- yl)ethan-1-one |
| 215 | 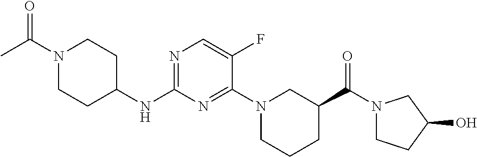 [SMILES: CC(=O)N1CCC(Nc2ncc(F)c(N3CCC[C@H](C(=O)N4CC[C@H](O)C4)C3)n2)CC1] | 1-(4-((5-fluoro-4-((S)-3- ((S)-3-hydroxypyrrolidine- 1-carbonyl)piperidin-1- yl)pyrimidin-2- yl)amino)piperidin-1- yl)ethan-1-one |
| 216 | 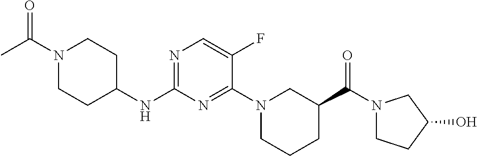 [SMILES: CC(=O)N1CCC(Nc2ncc(F)c(N3CCC[C@H](C(=O)N4CC[C@@H](O)C4)C3)n2)CC1] | 1-(4-((5-fluoro-4-((S)-3- ((R)-3-hydroxypyrrolidine- 1-carbonyl)piperidin-1- yl)pyrimidin-2- yl)amino)piperidin-1- yl)ethan-1-one |
| 217 | 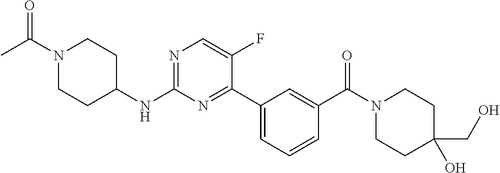 [SMILES: CC(=O)N1CCC(Nc2ncc(F)c(-c3cccc(C(=O)N4CCC(O)(CO)CC4)c3)n2)CC1] | 1-[4-[[5-fluoro-4-[3-[4- hydroxy-4- (hydroxymethyl)piperidine- 1- carbonyl]phenyl]pyrimidin- 2-yl]amino]-1- piperidyl]ethanone |
| 218 | 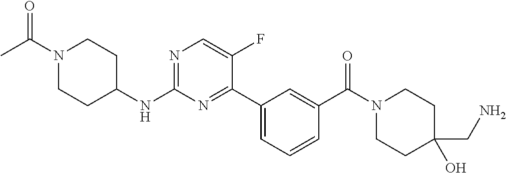 [SMILES: CC(=O)N1CCC(Nc2ncc(F)c(-c3cccc(C(=O)N4CCC(O)(CN)CC4)c3)n2)CC1] | 1-(4-((4-(3-(4- (aminomethyl)-4- hydroxypiperidine-1- carbonyl)phenyl)-5- fluoropyrimidin-2- yl)amino)piperidin-1- yl)ethan-1-one |
| 219 | 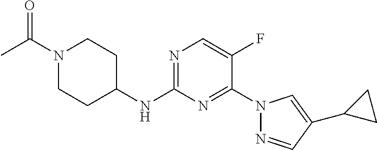 [SMILES: CC(=O)N1CCC(Nc2ncc(F)c(-n3cc(C4CC4)cn3)n2)CC1] | 1-(4-((4-(4-cyclopropyl- 1H-pyrazol-1-yl)-5- fluoropyrimidin-2- yl)amino)piperidin-1- yl)ethan-1-one |
| 220 | 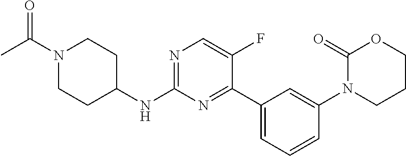 [SMILES: CC(=O)N1CCC(Nc2ncc(F)c(-c3cccc(N4CCCOC4=O)c3)n2)CC1] | 3-[3-[2-[(1-acetyl-4- piperidyl)amino]-5-fluoro- pyrimidin-4-yl]phenyl]- 1,3-oxazinan-2-one |
| 221 | 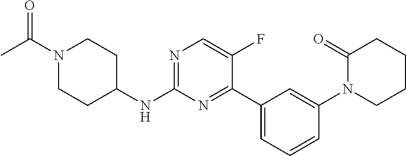 [SMILES: CC(=O)N1CCC(Nc2ncc(F)c(-c3cccc(N4CCCCC4=O)c3)n2)CC1] | 1-(3-(2-((1-acetylpiperidin- 4-yl)amino)-5- fluoropyrimidin-4- yl)phenyl)piperidin-2-one |
| 222 | 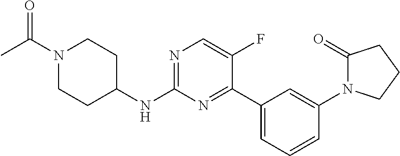 [SMILES: CC(=O)N1CCC(Nc2ncc(F)c(-c3cccc(N4CCCC4=O)c3)n2)CC1] | 1-[3-[2-[(1-acetyl-4- piperidyl)amino]-5-fluoro- pyrimidin-4- yl]phenyl]pyrrolidin-2-one |
| 223 | 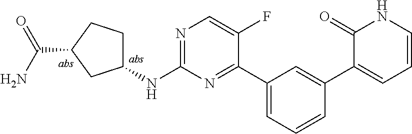 [SMILES: NC(=O)[C@@H]1CC[C@H](Nc2ncc(F)c(-c3cccc(-c4ccc[nH]c4=O)c3)n2)C1] | (1R,3S)-3-((5-fluoro-4-(3- (2-oxo-1,2-dihydropyridin- 3-yl)phenyl)pyrimidin-2- yl)amino)cyclopentane-1- carboxamide |
| 224 | 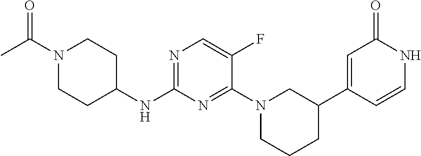 [SMILES: CC(=O)N1CCC(Nc2ncc(F)c(N3CCCC(c4cc[nH]c(=O)c4)C3)n2)CC1] | 4-(1-(2-((1-acetylpiperidin- 4-yl)amino)-5- fluoropyrimidin-4- yl)piperidin-3-yl)pyridin- 2(1H)-one |
| 225 | 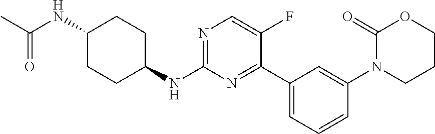 [SMILES: CC(=O)N[C@H]1CC[C@H](Nc2ncc(F)c(-c3cccc(N4CCCOC4=O)c3)n2)CC1] | trans-N-(4-((5-fluoro-4-(3- (2-oxo-1,3-oxazinan-3- yl)phenyl)pyrimidin-2- yl)amino)cyclohexyl) acetamide |
| 226 | 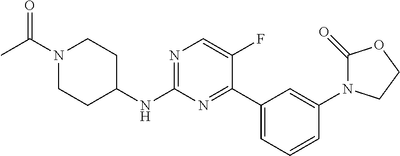 [SMILES: CC(=O)N1CCC(Nc2ncc(F)c(-c3cccc(N4CCOC4=O)c3)n2)CC1] | 3-(3-(2-((1-acetylpiperidin- 4-yl)amino)-5- fluoropyrimidin-4- yl)phenyl)oxazolidin-2-one |
| 227 | 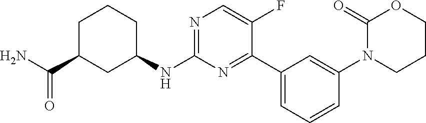 [SMILES: NC(=O)[C@H]1CCC[C@@H](Nc2ncc(F)c(-c3cccc(N4CCCOC4=O)c3)n2)C1] | cis-3-((5-fluoro-4-(3-(2- oxo-1,3-oxazinan-3- yl)phenyl)pyrimidin-2- yl)amino)cyclohexane-1- carboxamide |
| 228 | 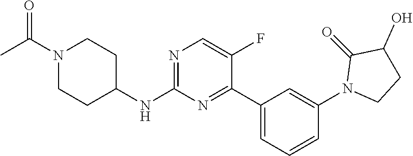 [SMILES: CC(=O)N1CCC(Nc2ncc(F)c(-c3cccc(N4CCC(O)C4=O)c3)n2)CC1] | 1-(3-(2-((1-acetylpiperidin- 4-yl)amino)-5- fluoropyrimidin-4- yl)phenyl)-3- hydroxypyrrolidin-2-one |
| 229 | 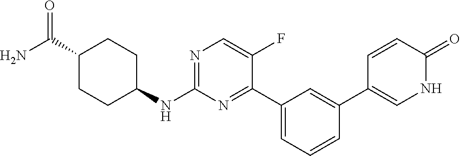 [SMILES: NC(=O)[C@H]1CC[C@H](Nc2ncc(F)c(-c3cccc(-c4ccc(=O)[nH]c4)c3)n2)CC1] | trans-4-((5-fluoro-4-(3-(6- oxo-1,6-dihydropyridin-3- yl)phenyl)pyrimidin-2- yl)amino)cyclohexane-1- carboxamide |
| 230 | 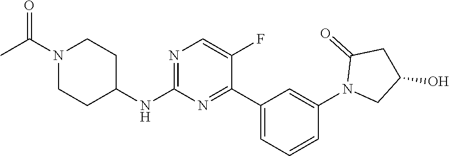 [SMILES: CC(=O)N1CCC(Nc2ncc(F)c(-c3cccc(N4C[C@@H](O)CC4=O)c3)n2)CC1] | (4S)-1-[3-[2-[(1-acetyl-4- piperidyl)amino]-5-fluoro- pyrimidin-4-yl]phenyl]-4- hydroxy-pyrrolidin-2-one |
| 231 | 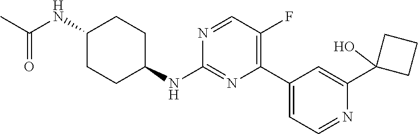 [SMILES: CC(=O)N[C@H]1CC[C@H](Nc2ncc(F)c(-c3ccnc(C4(O)CCC4)c3)n2)CC1] | trans-N-(4-((5-fluoro-4-(2- (1- hydroxycyclobutyl)pyridin- 4-yl)pyrimidin-2- yl)amino)cyclohexyl) acetamide |
| 232 | 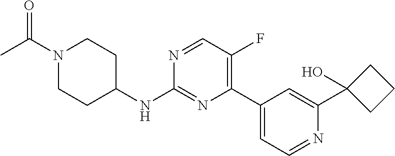 [SMILES: CC(=O)N1CCC(Nc2ncc(F)c(-c3ccnc(C4(O)CCC4)c3)n2)CC1] | 1-(4-((5-fluoro-4-(2-(1- hydroxycyclobutyl)pyridin- 4-yl)pyrimidin-2- yl)amino)piperidin-1- yl)ethan-1-one |
| 233 | 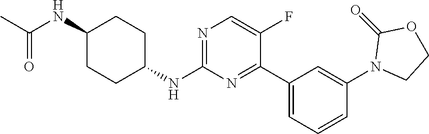 [SMILES: CC(=O)N[C@H]1CC[C@H](Nc2ncc(F)c(-c3cccc(N4CCOC4=O)c3)n2)CC1] | trans-N-(4-((5-fluoro-4-(3- (2-oxooxazolidin-3- yl)phenyl)pyrimidin-2- yl)amino)cyclohexyl) acetamide |
| 234 | 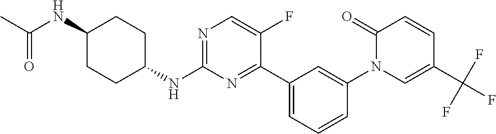 [SMILES: CC(=O)N[C@H]1CC[C@H](Nc2ncc(F)c(-c3cccc(-n4cc(C(F)(F)F)ccc4=O)c3)n2)CC1] | trans-N-(4-((5-fluoro-4-(3- (2-oxo-5- (trifluoromethyl)pyridin- 1(2H)- yl)phenyl)pyrimidin-2- yl)amino)cyclohexyl) acetamide |
| 235 | 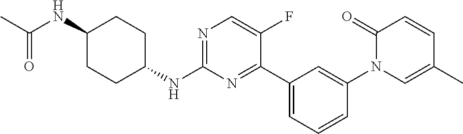 [SMILES: CC(=O)N[C@H]1CC[C@H](Nc2ncc(F)c(-c3cccc(-n4cc(C)ccc4=O)c3)n2)CC1] | trans-N-(4-((5-fluoro-4-(3- (5-methyl-2-oxopyridin- 1(2H)- yl)phenyl)pyrimidin-2- yl)amino)cyclohexyl) acetamide |
| 236 | 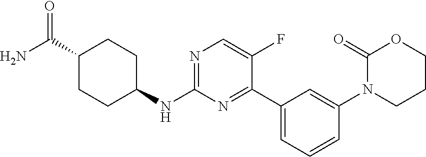 [SMILES: NC(=O)[C@H]1CC[C@H](Nc2ncc(F)c(-c3cccc(N4CCCOC4=O)c3)n2)CC1] | trans-4-((5-fluoro-4-(3-(2- oxo-1,3-oxazinan-3- yl)phenyl)pyrimidin-2- yl)amino)cyclohexane-1- carboxamide |
| 237 | 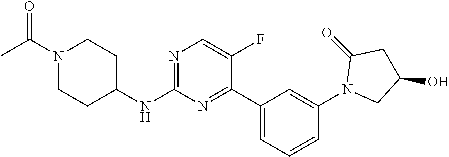 [SMILES: CC(=O)N1CCC(Nc2ncc(F)c(-c3cccc(N4C[C@H](O)CC4=O)c3)n2)CC1] | (4R)-1-[3-[2-[(1-acetyl-4- piperidyl)amino]-5-fluoro- pyrimidin-4-yl]phenyl]-4- hydroxy-pyrrolidin-2-one |
| 238 | 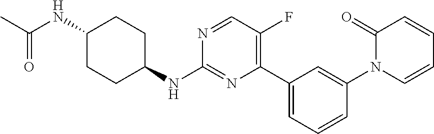 [SMILES: CC(=O)N[C@H]1CC[C@H](Nc2ncc(F)c(-c3cccc(-n4ccccc4=O)c3)n2)CC1] | trans-N-[4-[[5-fluoro-4-[3- (2-oxo-1- pyridyl)phenyl]pyrimidin- 2-yl]amino]cyclohexyl] acetamide |
| 239 | 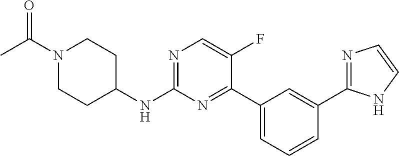 [SMILES: CC(=O)N1CCC(Nc2ncc(F)c(-c3cccc(-c4ncc[nH]4)c3)n2)CC1] | 1-(4-((4-(3-(1H-imidazol- 2-yl)phenyl)-5- fluoropyrimidin-2- yl)amino)piperidin-1- yl)ethan-1-one |
| 240 | 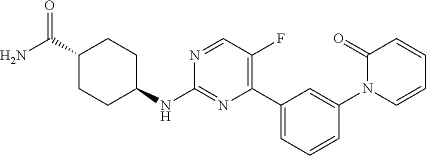 [SMILES: NC(=O)[C@H]1CC[C@H](Nc2ncc(F)c(-c3cccc(-n4ccccc4=O)c3)n2)CC1] | trans-(1r,4r)-4-((5-fluoro- 4-(3-(2-oxopyridin-1(2H)- yl)phenyl)pyrimidin-2- yl)amino)cyclohexane-1- carboxamide |
| 241 | 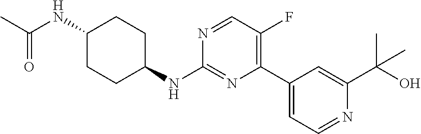 [SMILES: CC(=O)N[C@H]1CC[C@H](Nc2ncc(F)c(-c3ccnc(C(C)(C)O)c3)n2)CC1] | trans-N-(4-((5-fluoro-4-(2- (2-hydroxypropan-2- yl)pyridin-4-yl)pyrimidin- 2-yl)amino)cyclohexyl) acetamide |
| 242 | 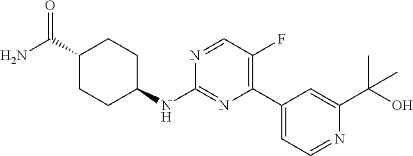 [SMILES: CC(C)(O)c1cc(-c2nc(N[C@H]3CC[C@H](C(N)=O)CC3)ncc2F)ccn1] | trans-4-((5-fluoro-4-(2-(2- hydroxypropan-2- yl)pyridin-4-yl)pyrimidin- 2-yl)amino)cyclohexane-1- carboxamide |
| 243 | 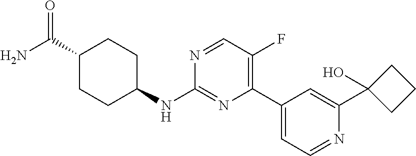 [SMILES: NC(=O)[C@H]1CC[C@H](Nc2ncc(F)c(-c3ccnc(C4(O)CCC4)c3)n2)CC1] | trans-4-((5-fluoro-4-(2-(1- hydroxycyclobutyl)pyridin- 4-yl)pyrimidin-2- yl)amino)cyclohexane-1- carboxamide |
| 244 | 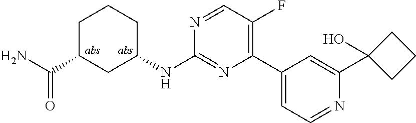 [SMILES: NC(=O)[C@@H]1CCC[C@H](Nc2ncc(F)c(-c3ccnc(C4(O)CCC4)c3)n2)C1] | (1R,3S)-3-((5-fluoro-4-(2- (1- hydroxycyclobutyl)pyridin- 4-yl)pyrimidin-2- yl)amino)cyclohexane-1- carboxamide |
| 245 | 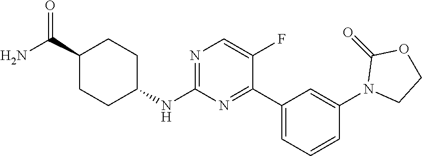 [SMILES: NC(=O)[C@H]1CC[C@H](Nc2ncc(F)c(-c3cccc(N4CCOC4=O)c3)n2)CC1] | trans-4-((5-fluoro-4-(3-(2- oxooxazolidin-3- yl)phenyl)pyrimidin-2- yl)amino)cyclohexane-1- carboxamide |
| 246 | 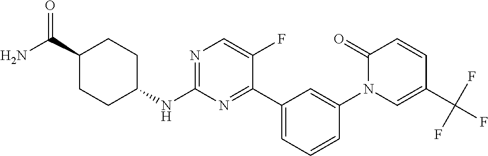 [SMILES: NC(=O)[C@H]1CC[C@H](Nc2ncc(F)c(-c3cccc(-n4cc(C(F)(F)F)ccc4=O)c3)n2)CC1] | trans-4-((5-fluoro-4-(3-(2- oxo-5- (trifluoromethyl)pyridin- 1(2H)-yl)phenyl) pyrimidin-2-yl)amino) cyclohexane-1- carboxamide |
| 247 | 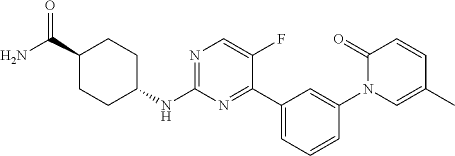 [SMILES: Cc1ccc(=O)n(-c2cccc(-c3nc(N[C@H]4CC[C@H](C(N)=O)CC4)ncc3F)c2)c1] | trans-4-((5-fluoro-4-(3-(5- methyl-2-oxopyridin- 1(2H)- yl)phenyl)pyrimidin-2- yl)amino)cyclohexane-1- carboxamide |
| 248 | 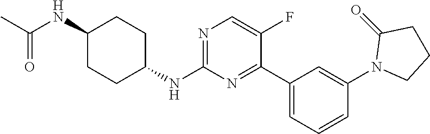 [SMILES: CC(=O)N[C@H]1CC[C@H](Nc2ncc(F)c(-c3cccc(N4CCCC4=O)c3)n2)CC1] | trans-N-(4-((5-fluoro-4-(3- (2-oxopyrrolidin-1- yl)phenyl)pyrimidin-2- yl)amino)cyclohexyl) acetamide |
| 249 | 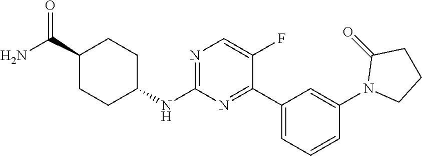 [SMILES: NC(=O)[C@H]1CC[C@H](Nc2ncc(F)c(-c3cccc(N4CCCC4=O)c3)n2)CC1] | trans-4-((5-fluoro-4-(3-(2- oxopyrrolidin-1- yl)phenyl)pyrimidin-2- yl)amino)cyclohexane-1- carboxamide |
| 250 | 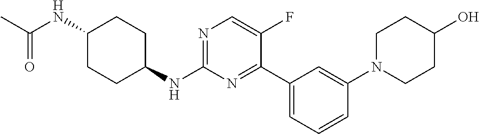 [SMILES: CC(=O)N[C@H]1CC[C@H](Nc2ncc(F)c(-c3cccc(N4CCC(O)CC4)c3)n2)CC1] | trans-N-(4-((5-fluoro-4-(3- (4-hydroxypiperidin-1- yl)phenyl)pyrimidin-2- yl)amino)cyclohexyl) acetamide |
| 251 | 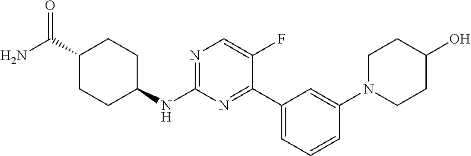 [SMILES: NC(=O)[C@H]1CC[C@H](Nc2ncc(F)c(-c3cccc(N4CCC(O)CC4)c3)n2)CC1] | trans-4-((5-fluoro-4-(3-(4- hydroxypiperidin-1- yl)phenyl)pyrimidin-2- yl)amino)cyclohexane-1- carboxamide |
| 252 | 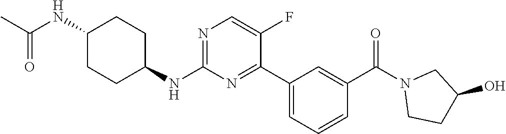 [SMILES: CC(=O)N[C@H]1CC[C@H](Nc2ncc(F)c(-c3cccc(C(=O)N4CC[C@H](O)C4)c3)n2)CC1] | trans-N-[4-[[5-fluoro-4-[3- [(3S)-3- hydroxypyrrolidine-1- carbonyl]phenyl] pyrimidin-2-yl]amino] cyclohexyl]acetamide |
| 253 | 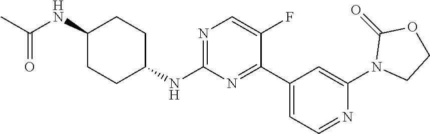 [SMILES: CC(=O)N[C@H]1CC[C@H](Nc2ncc(F)c(-c3ccnc(N4CCOC4=O)c3)n2)CC1] | trans-N-((1r,4r)-4-((5- fluoro-4-(2-(2- oxooxazolidin-3- yl)pyridin-4-yl)pyrimidin- 2-yl)amino)cyclohexyl) acetamide |
| 254 | 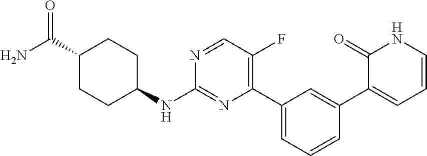 [SMILES: NC(=O)[C@H]1CC[C@H](Nc2ncc(F)c(-c3cccc(-c4ccc[nH]c4=O)c3)n2)CC1] | trans-(1r,4r)-4-((5-fluoro- 4-(3-(2-oxo-1,2- dihydropyridin-3- yl)phenyl)pyrimidin-2- yl)amino)cyclohexane-1- carboxamide |
| 255 | 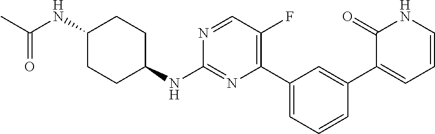 [SMILES: CC(=O)N[C@H]1CC[C@H](Nc2ncc(F)c(-c3cccc(-c4ccc[nH]c4=O)c3)n2)CC1] | N-(trans-4-((5-fluoro-4-(3- (2-oxo-1,2-dihydropyridin- 3-yl)phenyl)pyrimidin-2- yl)amino)cyclohexyl) acetamide |
| 256 | 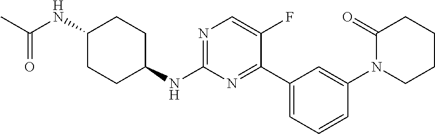 [SMILES: CC(=O)N[C@H]1CC[C@H](Nc2ncc(F)c(-c3cccc(N4CCCCC4=O)c3)n2)CC1] | trans-N-(4-((5-fluoro-4-(3- (2-oxopiperidin-1- yl)phenyl)pyrimidin-2- yl)amino)cyclohexyl) acetamide |
| 257 | 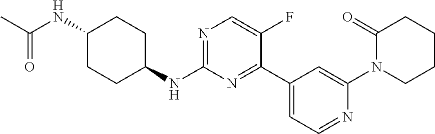 [SMILES: CC(=O)N[C@H]1CC[C@H](Nc2ncc(F)c(-c3ccnc(N4CCCCC4=O)c3)n2)CC1] | trans-N-(-4-((5-fluoro-4-(2- (2-oxopiperidin-1- yl)pyridin-4-yl)pyrimidin- 2-yl)amino)cyclohexyl) acetamide |
| 258 | 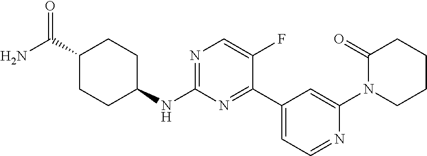 [SMILES: NC(=O)[C@H]1CC[C@H](Nc2ncc(F)c(-c3ccnc(N4CCCCC4=O)c3)n2)CC1] | trans-4-((5-fluoro-4-(2-(2- oxopiperidin-1-yl)pyridin- 4-yl)pyrimidin-2- yl)amino)cyclohexane-1- carboxamide |
| 259 | 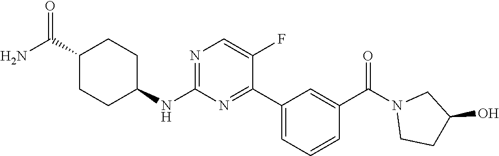 [SMILES: NC(=O)[C@H]1CC[C@H](Nc2ncc(F)c(-c3cccc(C(=O)N4CC[C@H](O)C4)c3)n2)CC1] | trans-(S)-4-((5-fluoro-4-(3- (3-hydroxypyrrolidine-1- carbonyl)phenyl)pyrimidin- 2-yl)amino)cyclohexane- 1-carboxamide |
| 260 | 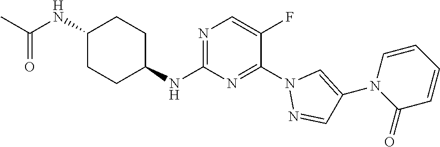 [SMILES: CC(=O)N[C@H]1CC[C@H](Nc2ncc(F)c(-n3cc(-n4ccccc4=O)cn3)n2)CC1] | trans-N-((1r,4r)-4-((5- fluoro-4-(4-(2-oxopyridin- 1(2H)-yl)-1H-pyrazol-1- yl)pyrimidin-2- yl)amino)cyclohexyl) acetamide |
| 261 | 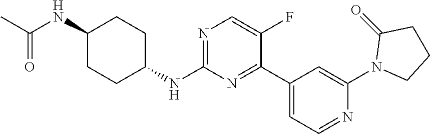 [SMILES: CC(=O)N[C@H]1CC[C@H](Nc2ncc(F)c(-c3ccnc(N4CCCC4=O)c3)n2)CC1] | trans-N-(4-((5-fluoro-4-(2- (2-oxopyrrolidin-1- yl)pyridin-4-yl)pyrimidin- 2-yl)amino)cyclohexyl) acetamide |
| 262 | 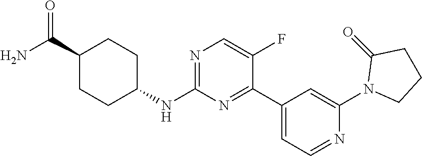 [SMILES: NC(=O)[C@H]1CC[C@H](Nc2ncc(F)c(-c3ccnc(N4CCCC4=O)c3)n2)CC1] | trans-(1r,4r)-4-((5-fluoro- 4-(2-(2-oxopyrrolidin-1- yl)pyridin-4-yl)pyrimidin- 2-yl)amino)cyclohexane- 1-carboxamide |
| 263 | 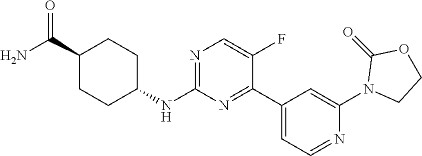 [SMILES: NC(=O)[C@H]1CC[C@H](Nc2ncc(F)c(-c3ccnc(N4CCOC4=O)c3)n2)CC1] | trans-4-((5-fluoro-4-(2-(2- oxooxazolidin-3- yl)pyridin-4-yl)pyrimidin- 2-yl)amino)cyclohexane-1- carboxamide |
| 264 | 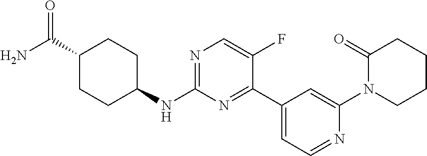 [SMILES: NC(=O)[C@H]1CC[C@H](Nc2ncc(F)c(-c3ccnc(N4CCCCC4=O)c3)n2)CC1] | trans-4-((5-fluoro-4-(3-(2- oxopiperidin-1- yl)phenyl)pyrimidin-2- yl)amino)cyclohexane-1- carboxamide |
| 265 | 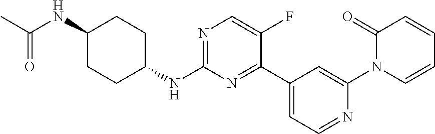 [SMILES: CC(=O)N[C@H]1CC[C@H](Nc2ncc(F)c(-c3ccnc(-n4ccccc4=O)c3)n2)CC1] | trans-N-(4-((5-fluoro-4-(2- oxo-2H-[1,2′-bipyridin]-4′- yl)pyrimidin-2- yl)amino)cyclohexyl) acetamide |
| 266 | 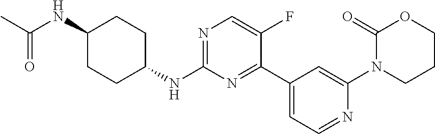 [SMILES: CC(=O)N[C@H]1CC[C@H](Nc2ncc(F)c(-c3ccnc(N4CCCOC4=O)c3)n2)CC1] | trans-N-(4-((5-fluoro-4-(2- (2-oxo-1,3-oxazinan-3- yl)pyridin-4-yl)pyrimidin- 2-yl)amino)cyclohexyl) acetamide |
| 267 | 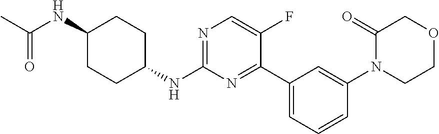 [SMILES: CC(=O)N[C@H]1CC[C@H](Nc2ncc(F)c(-c3cccc(N4CCOCC4=O)c3)n2)CC1] | trans-N-(4-((5-fluoro-4-(3- (3- oxomorpholino)phenyl) pyrimidin-2-yl)amino) cyclohexyl)acetamide |
| 268 | 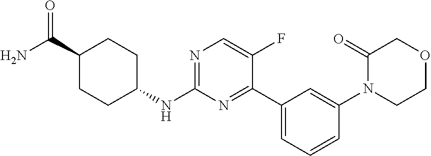 [SMILES: NC(=O)[C@H]1CC[C@H](Nc2ncc(F)c(-c3cccc(N4CCOCC4=O)c3)n2)CC1] | trans-4-((5-fluoro-4-(3-(3- oxomorpholino)phenyl) pyrimidin-2-yl)amino) cyclohexane-1- carboxamide |
| 269 | 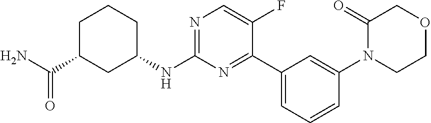 [SMILES: NC(=O)[C@@H]1CCC[C@H](Nc2ncc(F)c(-c3cccc(N4CCOCC4=O)c3)n2)C1] | cis-3-((5-fluoro-4-(3-(3- oxomorpholino)phenyl) pyrimidin-2-yl) amino)cyclohexane-1- carboxamide |
| 270 | 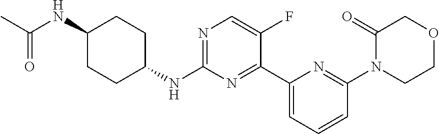 [SMILES: CC(=O)N[C@H]1CC[C@H](Nc2ncc(F)c(-c3cccc(N4CCOCC4=O)n3)n2)CC1] | trans-N-(4-((5-fluoro-4-(6- (3-oxomorpholino)pyridin- 2-yl)pyrimidin-2- yl)amino)cyclohexyl) acetamide |
| 271 | 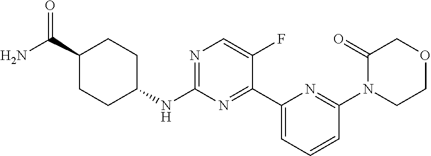 [SMILES: NC(=O)[C@H]1CC[C@H](Nc2ncc(F)c(-c3cccc(N4CCOCC4=O)n3)n2)CC1] | trans-4-((5-fluoro-4-(6-(3- oxomorpholino)pyridin-3- yl)pyrimidin-2- yl)amino)cyclohexane-1- carboxamide |
| 272 | 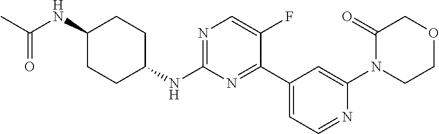 [SMILES: CC(=O)N[C@H]1CC[C@H](Nc2ncc(F)c(-c3ccnc(N4CCOCC4=O)c3)n2)CC1] | trans-N-(4-((5-fluoro-4-(2- (3-oxomorpholino)pyridin- 4-yl)pyrimidin-2- yl)amino)cyclohexyl) acetamide |
| 273 | 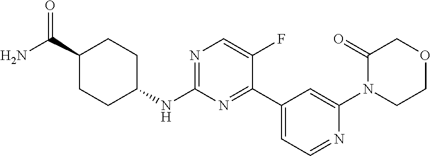 [SMILES: NC(=O)[C@H]1CC[C@H](Nc2ncc(F)c(-c3ccnc(N4CCOCC4=O)c3)n2)CC1] | trans-4-((5-fluoro-4-(2-(3- oxomorpholino)pyridin-4- yl)pyrimidin-2- yl)amino)cyclohexane-1- carboxamide |
| 274 | 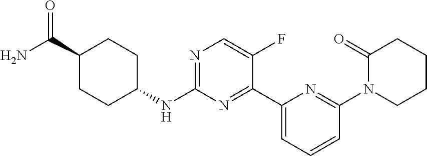 [SMILES: NC(=O)[C@H]1CC[C@H](Nc2ncc(F)c(-c3cccc(N4CCCCC4=O)n3)n2)CC1] | trans-4-((5-fluoro-4-(6-(2- oxopiperidin-1-yl)pyridin- 2-yl)pyrimidin-2- yl)amino)cyclohexane-1- carboxamide |
| 606 | 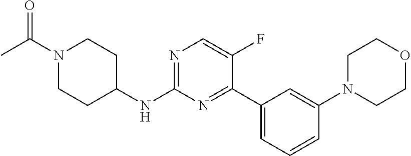 [SMILES: CC(=O)N1CCC(Nc2ncc(F)c(-c3cccc(N4CCOCC4)c3)n2)CC1] | 1-[4-[[5-fluoro-4-(3- morpholinophenyl) pyrimidin-2-yl]amino]-1- piperidyl]ethanone |
| 607 | 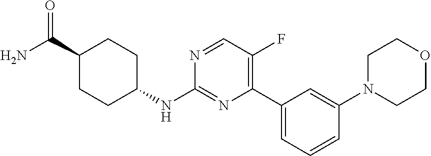 [SMILES: NC(=O)[C@H]1CC[C@H](Nc2ncc(F)c(-c3cccc(N4CCOCC4)c3)n2)CC1] | trans-4-((5-fluoro-4-(3- morpholinophenyl) pyrimidin-2-yl)amino) cyclohexane-1- carboxamide |
| 608 | 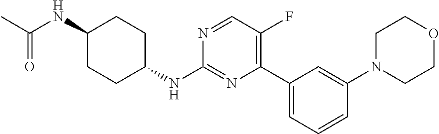 [SMILES: CC(=O)N[C@H]1CC[C@H](Nc2ncc(F)c(-c3cccc(N4CCOCC4)c3)n2)CC1] | trans-N-(4-((5-fluoro-4-(3- morpholinophenyl) pyrimidin-2-yl)amino) cyclohexyl)acetamide |
| 609 | 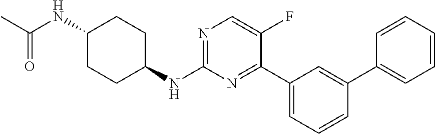 [SMILES: CC(=O)N[C@H]1CC[C@H](Nc2ncc(F)c(-c3cccc(-c4ccccc4)c3)n2)CC1] | trans-N-(4-((4-([1,1′- biphenyl]-3-yl)-5- fluoropyrimidin-2- yl)amino)cyclohexyl) acetamide |
| 610 | 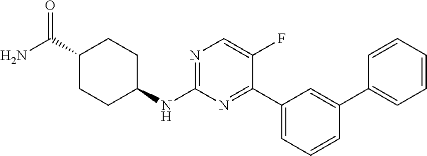 [SMILES: NC(=O)[C@H]1CC[C@H](Nc2ncc(F)c(-c3cccc(-c4ccccc4)c3)n2)CC1] | trans-4-((4-([1,1′- biphenyl]-3-yl)-5- fluoropyrimidin-2- yl)amino)cyclohexane-1- carboxamide |
| 611 | 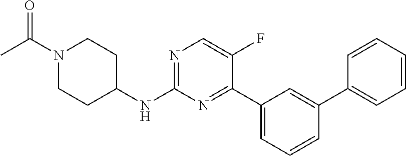 [SMILES: CC(=O)N1CCC(Nc2ncc(F)c(-c3cccc(-c4ccccc4)c3)n2)CC1] | 1-(4-((4-([1,1′-biphenyl]-3- yl)-5-fluoropyrimidin-2- yl)amino)piperidin-1- yl)ethan-1-one |
| 612 | 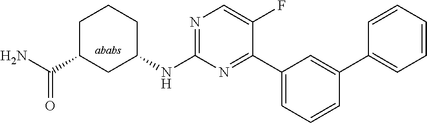 [SMILES: NC(=O)[C@@H]1CCC[C@H](Nc2ncc(F)c(-c3cccc(-c4ccccc4)c3)n2)C1] | (1R,3S)-3-((4-([1,1′- biphenyl]-3-yl)-5- fluoropyrimidin-2-yl) amino)cyclohexane-1- carboxamide |
| 613 | 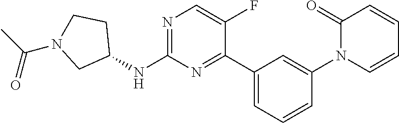 [SMILES: CC(=O)N1CC[C@H](Nc2ncc(F)c(-c3cccc(-n4ccccc4=O)c3)n2)C1] | (S)-1-(3-(2-((1- acetylpyrrolidin-3- yl)amino)-5- fluoropyrimidin-4- yl)phenyl)pyridin-2(1H)- one |
| 614 | 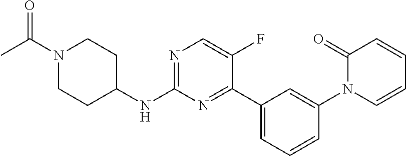 [SMILES: CC(=O)N1CCC(Nc2ncc(F)c(-c3cccc(-n4ccccc4=O)c3)n2)CC1] | 1-(3-(2-((1-acetylpiperidin- 4-yl)amino)-5- fluoropyrimidin-4- yl)phenyl)pyridin-2(1H)- one |
| 615 | 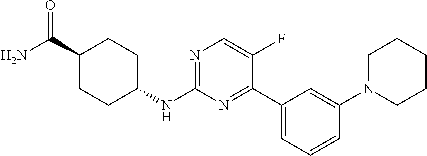 [SMILES: NC(=O)[C@H]1CC[C@H](Nc2ncc(F)c(-c3cccc(N4CCCCC4)c3)n2)CC1] | trans-4-((5-fluoro-4-(3- (piperidin-1- yl)phenyl)pyrimidin-2- yl)amino)cyclohexane-1- carboxamide |
| 616 | 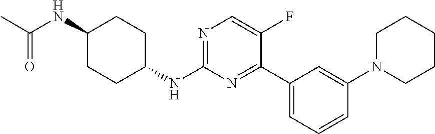 [SMILES: CC(=O)N[C@H]1CC[C@H](Nc2ncc(F)c(-c3cccc(N4CCCCC4)c3)n2)CC1] | trans-N-(4-((5-fluoro-4-(3- (piperidin-1- yl)phenyl)pyrimidin-2- yl)amino)cyclohexyl) acetamide |
| 617 | 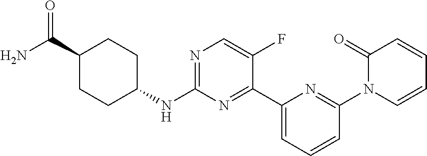 [SMILES: NC(=O)[C@H]1CC[C@H](Nc2ncc(F)c(-c3cccc(-n4ccccc4=O)n3)n2)CC1] | trans-4-((5-fluoro-4-(2- oxo-2H-[1,2′-bipyridin]-6′- yl)pyrimidin-2- yl)amino)cyclohexane-1- carboxamide |
| 618 | 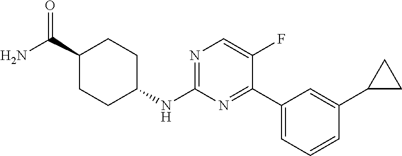 [SMILES: NC(=O)[C@H]1CC[C@H](Nc2ncc(F)c(-c3cccc(C4CC4)c3)n2)CC1] | trans-(1r,4r)-4-((4-(3- cyclopropylphenyl)-5- fluoropyrimidin-2- yl)amino)cyclohexane-1- carboxamide |
| 619 | 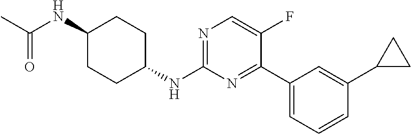 [SMILES: CC(=O)N[C@H]1CC[C@H](Nc2ncc(F)c(-c3cccc(C4CC4)c3)n2)CC1] | trans-N-((1r,4r)-4-((4-(3- cyclopropylphenyl)-5- fluoropyrimidin-2- yl)amino)cyclohexyl) acetamide |
| 620 | 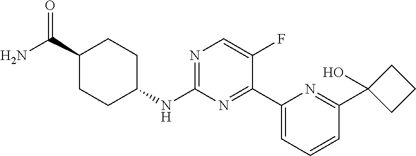 [SMILES: NC(=O)[C@H]1CC[C@H](Nc2ncc(F)c(-c3cccc(C4(O)CCC4)n3)n2)CC1] | trans-4-((5-fluoro-4-(6-(1- hydroxycyclobutyl)pyridin- 2-yl)pyrimidin-2- yl)amino)cyclohexane-1- carboxamide |
| 621 | 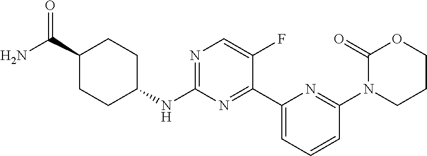 [SMILES: NC(=O)[C@H]1CC[C@H](Nc2ncc(F)c(-c3cccc(N4CCCOC4=O)n3)n2)CC1] | trans-4-((5-fluoro-4-(6-(2- oxo-1,3-oxazinan-3- yl)pyridin-2-yl)pyrimidin- 2-yl)amino)cyclohexane-1- carboxamide |
| 622 | 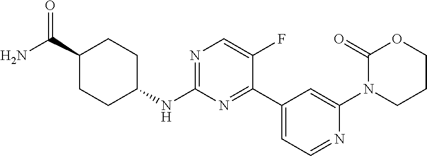 [SMILES: NC(=O)[C@H]1CC[C@H](Nc2ncc(F)c(-c3ccnc(N4CCCOC4=O)c3)n2)CC1] | trans-4-[[5-fluoro-4-[2-(2- oxo-1,3-oxazinan-3-yl)-4- pyridyl]pyrimidin-2- yl]amino]cyclohexane- carboxamide |
| 623 | 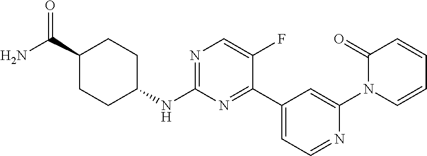 [SMILES: NC(=O)[C@H]1CC[C@H](Nc2ncc(F)c(-c3ccnc(-n4ccccc4=O)c3)n2)CC1] | trans-4-((5-fluoro-4-(2- oxo-2H-[1,2′-bipyridin]-4′- yl)pyrimidin-2- yl)amino)cyclohexane-1- carboxamide |
| 624 | 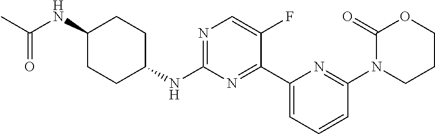 [SMILES: CC(=O)N[C@H]1CC[C@H](Nc2ncc(F)c(-c3cccc(N4CCCOC4=O)n3)n2)CC1] | N-(trans-4-((5-fluoro-4-(6- (2-oxo-1,3-oxazinan-3- yl)pyridin-2-yl)pyrimidin- 2-yl)amino)cyclohexyl) acetamide |
| 625 | 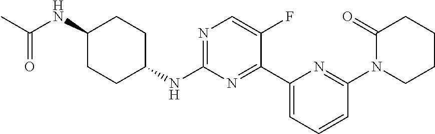 [SMILES: CC(=O)N[C@H]1CC[C@H](Nc2ncc(F)c(-c3cccc(N4CCCCC4=O)n3)n2)CC1] | N-(trans-4-((5-fluoro-4-(6- (2-oxopiperidin-1-yl) pyridin-2-yl)pyrimidin- 2-yl)amino)cyclohexyl) acetamide |
| 626 | 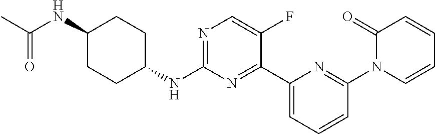 [SMILES: CC(=O)N[C@H]1CC[C@H](Nc2ncc(F)c(-c3cccc(-n4ccccc4=O)n3)n2)CC1] | trans-N-(4-((5-fluoro-4-(2- oxo-2H-[1,2′-bipyridin]-6′- yl)pyrimidin-2- yl)amino)cyclohexyl) acetamide |
| 627 | 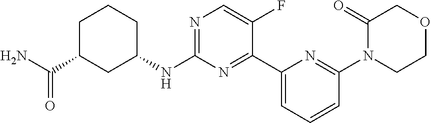 [SMILES: NC(=O)[C@@H]1CCC[C@H](Nc2ncc(F)c(-c3cccc(N4CCOCC4=O)n3)n2)C1] | cis-3-((5-fluoro-4-(6-(3- oxomorpholino)pyridin-2- yl)pyrimidin-2- yl)amino)cyclohexane-1- carboxamide |
| 628 | 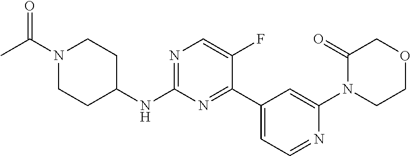 [SMILES: CC(=O)N1CCC(Nc2ncc(F)c(-c3ccnc(N4CCOCC4=O)c3)n2)CC1] | 4-(4-(2-((1-acetylpiperidin- 4-yl)amino)-5- fluoropyrimidin-4- yl)pyridin-2-yl)morpholin- 3-one |
| 629 | 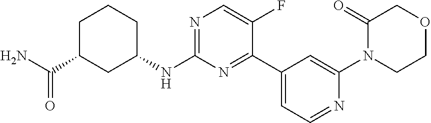 [SMILES: NC(=O)[C@@H]1CCC[C@H](Nc2ncc(F)c(-c3ccnc(N4CCOCC4=O)c3)n2)C1] | cis-3-((5-fluoro-4-(2-(3- oxomorpholino)pyridin-4- yl)pyrimidin-2- yl)amino)cyclohexane-1- carboxamide |
| 630 | 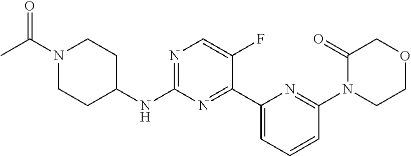 [SMILES: CC(=O)N1CCC(Nc2ncc(F)c(-c3cccc(N4CCOCC4=O)n3)n2)CC1] | 4-(6-(2-((1-acetylpiperidin- 4-yl)amino)-5- fluoropyrimidin-4- yl)pyridin-2-yl)morpholin- 3-one |
| 631 | 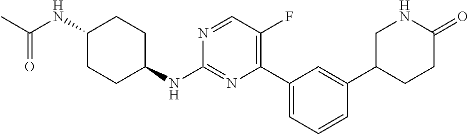 [SMILES: CC(=O)N[C@H]1CC[C@H](Nc2ncc(F)c(-c3cccc(C4CCC(=O)NC4)c3)n2)CC1] | N-(trans-4-((5-fluoro-4-(3- (6-oxopiperidin-3- yl)phenyl)pyrimidin-2- yl)amino)cyclohexyl) acetamide |

TABLE 2
Representative Compounds of Formula (I′), (I), and (IV)

| Co. No. | Structure and Name |
|---|---|
| 44 | 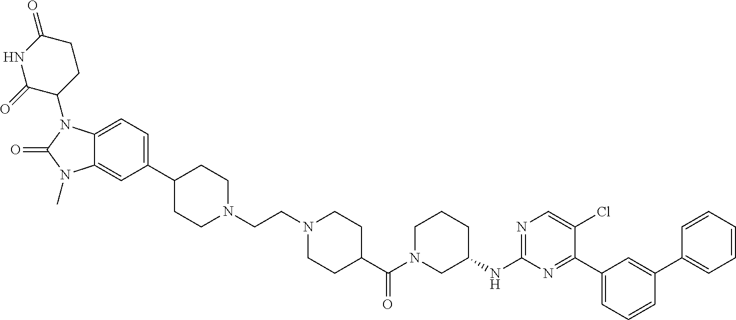 [SMILES: Cn1c(=O)n(C2CCC(=O)NC2=O)c2ccc(C3CCN(CCN4CCC(C(=O)N5CCC[C@H](Nc6ncc(Cl)c(-c7cccc(-c8ccccc8)c7)n6)C5)CC4)CC3)cc21] |
| | 3-(5-(1-(2-(4-((S)-3-((4-([1,1′-biphenyl]-3-yl)-5-chloropyrimidin-2-yl)amino)piperidine-1-carbonyl)piperidin-1-yl)ethyl)- |
| | piperidin-4-yl)-3-methyl-2-oxo-2,3-dihydro-1H-benzo[d]imidazol-1-yl)piperidine-2,6-dione |
| 45 | 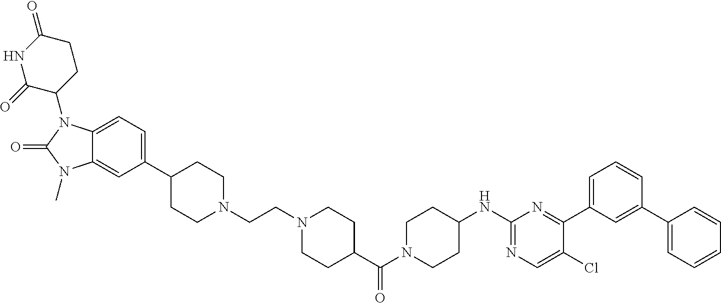 [SMILES: Cn1c(=O)n(C2CCC(=O)NC2=O)c2ccc(C3CCN(CCN4CCC(C(=O)N5CCC(Nc6ncc(Cl)c(-c7cccc(-c8ccccc8)c7)n6)CC5)CC4)CC3)cc21] |
| | 3-[5-[1-[2-[4-[4-[[5-chloro-4-(3-phenylphenyl)pyrimidin-2-yl]amino]piperidine-1-carbonyl]-1-piperidyl]ethyl]-4- |
| | piperidyl]-3-methyl-2-oxo-benzimidazol-1-yl]piperidine-2,6-dione |
| 46 | 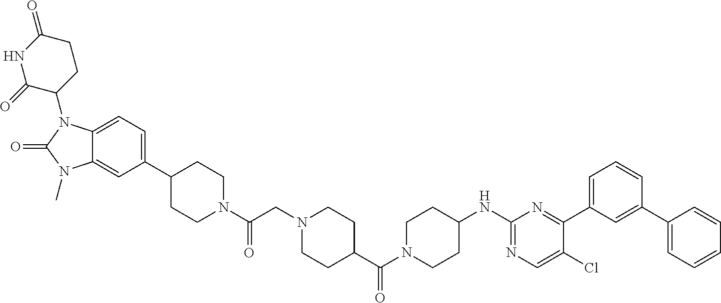 [SMILES: Cn1c(=O)n(C2CCC(=O)NC2=O)c2ccc(C3CCN(C(=O)CN4CCC(C(=O)N5CCC(Nc6ncc(Cl)c(-c7cccc(-c8ccccc8)c7)n6)CC5)CC4)CC3)cc21] |
| | 3-(5-(1-(2-(4-(4-((4-([1,1′-biphenyl]-3-yl)-5-chloropyrimidin-2-yl)amino)piperidine-1-carbonyl)piperidin-1-yl)acetyl)- |
| | piperidin-4-yl)-3-methyl-2-oxo-2,3-dihydro-1H-benzo[d]imidazol-1-yl)piperidine-2,6-dione |
| 47 | 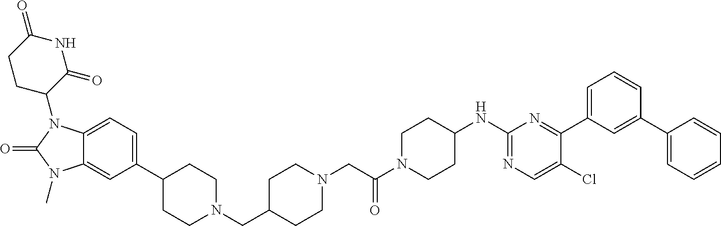 [SMILES: Cn1c(=O)n(C2CCC(=O)NC2=O)c2ccc(C3CCN(CC4CCN(CC(=O)N5CCC(Nc6ncc(Cl)c(-c7cccc(-c8ccccc8)c7)n6)CC5)CC4)CC3)cc21] |
| | 3-(5-(1-((1-(2-(4-((4-([1,1′-biphenyl]-3-yl)-5-chloropyrimidin-2-yl)amino)piperidin-1-yl)-2-oxoethyl)piperidin-4-yl)- |
| | methyl)piperidin-4-yl)-3-methyl-2-oxo-2,3-dihydro-1H-benzo[d]imidazol-1-yl)piperidine-2,6-dione |
| 48 | 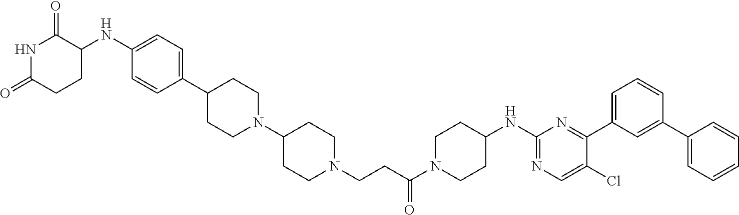 [SMILES: O=C1CCC(Nc2ccc(C3CCN(C4CCN(CCC(=O)N5CCC(Nc6ncc(Cl)c(-c7cccc(-c8ccccc8)c7)n6)CC5)CC4)CC3)cc2)C(=O)N1] |
| | 3-((4-(1′-(3-(4-((4-([1,1′-biphenyl]-3-yl)-5-chloropyrimidin-2-yl)amino)piperidin-1-yl)-3-oxopropyl)-[1,4′-bipiperidin]-4- |
| | yl)phenyl)amino)piperidine-2,6-dione |
| 49 | 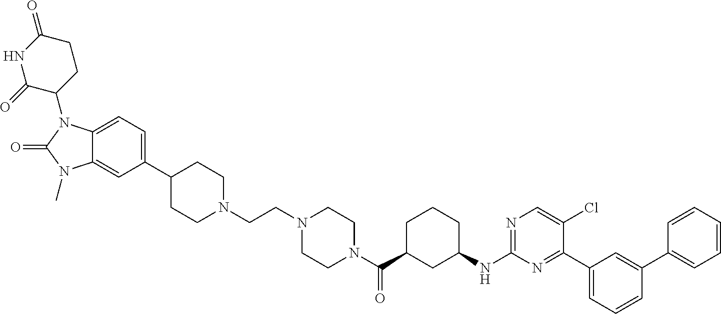 [SMILES: Cn1c(=O)n(C2CCC(=O)NC2=O)c2ccc(C3CCN(CCN4CCN(C(=O)[C@H]5CCC[C@@H](Nc6ncc(Cl)c(-c7cccc(-c8ccccc8)c7)n6)C5)CC4)CC3)cc21] |
| | cis-3-(5-(1-(2-(4-(3-((4-([1,1′-biphenyl]-3-yl)-5-chloropyrimidin-2-yl)amino)cyclohexane-1-carbonyl)piperazin-1-yl)- |
| | ethyl)piperidin-4-yl)-3-methyl-2-oxo-2,3-dihydro-1H-benzo[d]imidazol-1-yl)piperidine-2,6-dione |
| 50 | 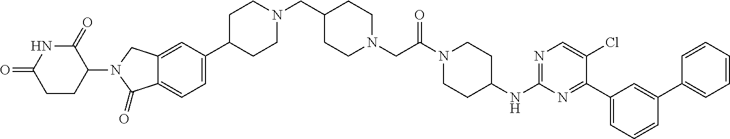 [SMILES: O=C1CCC(N2Cc3cc(C4CCN(CC5CCN(CC(=O)N6CCC(Nc7ncc(Cl)c(-c8cccc(-c9ccccc9)c8)n7)CC6)CC5)CC4)ccc3C2=O)C(=O)N1] |
| | 3-[5-[1-[[1-[2-[4-[5-chloro-4-(3-phenylphenyl)pyrimidin-2-yl]amino]-1-piperidyl]-2-oxo-ethyl]-4-piperidyl]methyl]-4- |
| | piperidyl]-1-oxo-indan-2-yl]piperidine-2,6-dione |
| 51 | 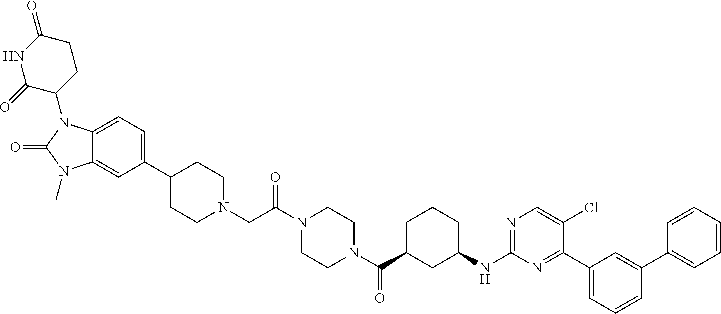 [SMILES: Cn1c(=O)n(C2CCC(=O)NC2=O)c2ccc(C3CCN(CC(=O)N4CCN(C(=O)[C@H]5CCC[C@@H](Nc6ncc(Cl)c(-c7cccc(-c8ccccc8)c7)n6)C5)CC4)CC3)cc21] |
| | cis-3-(5-(1-(2-(4-(3-((4-([1,1′-biphenyl]-3-yl)-5-chloropyrimidin-2-yl)amino)cyclohexane-1-carbonyl)piperazin-1-yl)- |
| | 2-oxoethyl)piperidin-4-yl)-3-methyl-2-oxo-2,3-dihydro-1H-benzo[d]imidazol-1-yl)piperidine-2,6-dione |
| 52 | 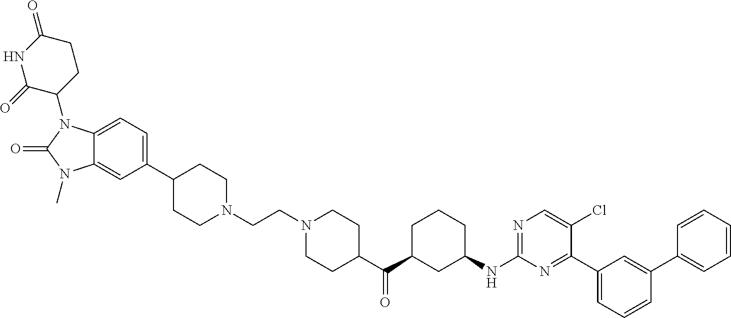 [SMILES: Cn1c(=O)n(C2CCC(=O)NC2=O)c2ccc(C3CCN(CCN4CCC(C(=O)[C@H]5CCC[C@@H](Nc6ncc(Cl)c(-c7cccc(-c8ccccc8)c7)n6)C5)CC4)CC3)cc21] |
| | cis-3-(5-(1-(2-(1-(3-((4-([1,1′-biphenyl]-3-yl)-5-chloropyrimidin-2-yl)amino)cyclohexane-1-carbonyl)piperidin-4-yl)- |
| | ethyl)piperidin-4-yl)-3-methyl-2-oxo-2,3-dihydro-1H-benzo[d]imidazol-1-yl)piperidine-2,6-dione |
| 53 | 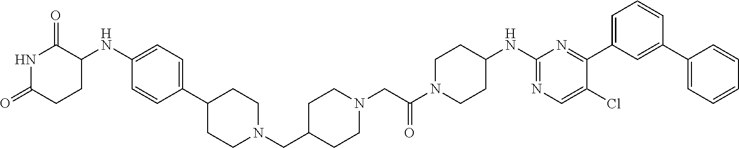 [SMILES: O=C1CCC(Nc2ccc(C3CCN(CC4CCN(CC(=O)N5CCC(Nc6ncc(Cl)c(-c7cccc(-c8ccccc8)c7)n6)CC5)CC4)CC3)cc2)C(=O)N1] |
| | 3-((4-(1-((1-(2-(4-((4-([1,1′-biphenyl]-3-yl)-5-chloropyrimidin-2-yl)amino)piperidin-1-yl)-2-oxoethyl)piperidin-4-yl)- |
| | methyl)piperidin-4-yl)phenyl)amino)piperidine-2,6-dione |
| 54 | 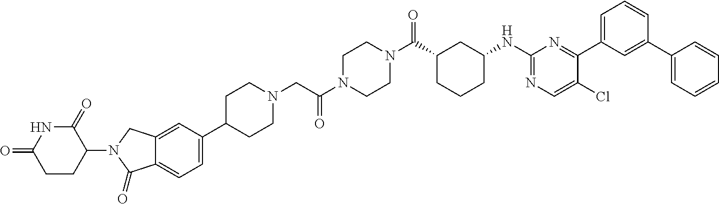 [SMILES: O=C1CCC(N2Cc3cc(C4CCN(CC(=O)N5CCN(C(=O)[C@H]6CCC[C@@H](Nc7ncc(Cl)c(-c8cccc(-c9ccccc9)c8)n7)C6)CC5)CC4)ccc3C2=O)C(=O)N1] |
| | cis-3-(5-(1-(2-(4-(3-((4-([1,1′-biphenyl]-3-yl)-5-chloropyrimidin-2-yl)amino)cyclohexane-1-carbonyl)piperazin-1-yl)- |
| | 2-oxoethyl)piperidin-4-yl)-1-oxoisoindolin-2-yl)piperidine-2,6-dione |
| 55 | 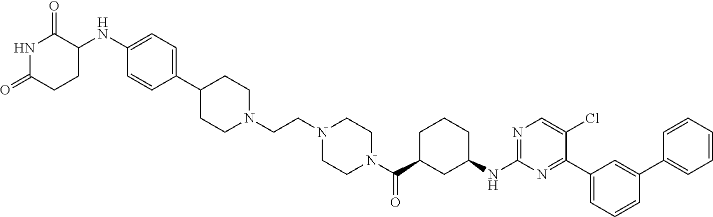 [SMILES: O=C1CCC(Nc2ccc(C3CCN(CCN4CCN(C(=O)[C@H]5CCC[C@@H](Nc6ncc(Cl)c(-c7cccc(-c8ccccc8)c7)n6)C5)CC4)CC3)cc2)C(=O)N1] |
| | cis-3-((4-(1-(2-(4-(3-((4-([1,1′-biphenyl]-3-yl)-5-chloropyrimidin-2-yl)amino)cyclohexane-1-carbonyl)piperazin-1- |
| | yl)ethyl)piperidin-4-yl)phenyl)amino)piperidine-2,6-dione |
| 56 | 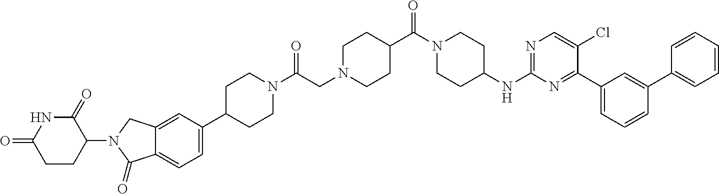 [SMILES: O=C1CCC(N2Cc3cc(C4CCN(C(=O)CN5CCC(C(=O)N6CCC(Nc7ncc(Cl)c(-c8cccc(-c9ccccc9)c8)n7)CC6)CC5)CC4)ccc3C2=O)C(=O)N1] |
| | 3-(5-(1-(2-(4-(4-((4-([1,1′-biphenyl]-3-yl)-5-chloropyrimidin-2-yl)amino)piperidine-1-carbonyl)piperidin-1-yl)acetyl)- |
| | piperidin-4-yl)-1-oxoisoindolin-2-yl)piperidine-2,6-dione |
| 57 | 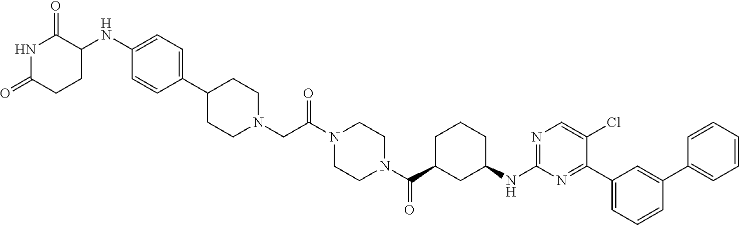 [SMILES: O=C1CCC(Nc2ccc(C3CCN(CC(=O)N4CCN(C(=O)[C@H]5CCC[C@@H](Nc6ncc(Cl)c(-c7cccc(-c8ccccc8)c7)n6)C5)CC4)CC3)cc2)C(=O)N1] |
| | cis-3-((4-(1-(2-(4-(3-((4-([1,1′-biphenyl]-3-yl)-5-chloropyrimidin-2-yl)amino)cyclohexane-1-carbonyl)piperazin-1-yl)- |
| | 2-oxoethyl)piperidin-4-yl)phenyl)amino)piperidine-2,6-dione |
| 58 | 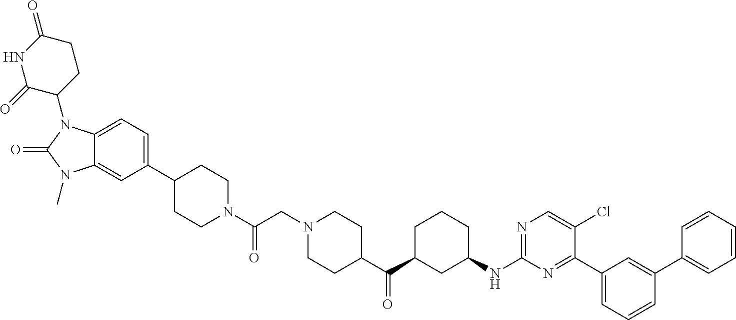 [SMILES: Cn1c(=O)n(C2CCC(=O)NC2=O)c2ccc(C3CCN(C(=O)CN4CCC(C(=O)[C@H]5CCC[C@@H](Nc6ncc(Cl)c(-c7cccc(-c8ccccc8)c7)n6)C5)CC4)CC3)cc21] |
| | cis-3-(5-(1-(2-(4-(3-((4-([1,1′-biphenyl]-3-yl)-5-chloropyrimidin-2-yl)amino)cyclohexane-1-carbonyl)piperazin-1-yl)- |
| | acetyl)piperidin-4-yl)-3-methyl-2-oxo-2,3-dihydro-1H-benzo[d]imidazol-1-yl)piperidine-2,6-dione |
| 59 | 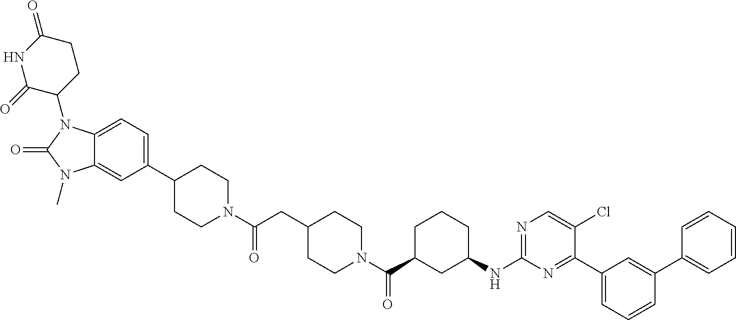 [SMILES: Cn1c(=O)n(C2CCC(=O)NC2=O)c2ccc(C3CCN(C(=O)CC4CCN(C(=O)[C@H]5CCC[C@@H](Nc6ncc(Cl)c(-c7cccc(-c8ccccc8)c7)n6)C5)CC4)CC3)cc21] |
| | cis-3-(5-(1-(2-(1-(3-((4-([1,1′-biphenyl]-3-yl)-5-chloropyrimidin-2-yl)amino)cyclohexane-1-carbonyl)piperidin-4-yl)- |
| | acetyl)piperidin-4-yl)-3-methyl-2-oxo-2,3-dihydro-1H-benzo[d]imidazol-1-yl)piperidine-2,6-dione |
| 60 | 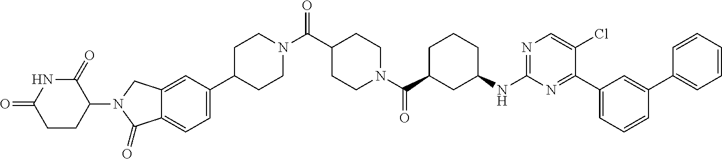 [SMILES: O=C1CCC(N2Cc3cc(C4CCN(C(=O)C5CCN(C(=O)[C@H]6CCC[C@@H](Nc7ncc(Cl)c(-c8cccc(-c9ccccc9)c8)n7)C6)CC5)CC4)ccc3C2=O)C(=O)N1] |
| | cis-3-(5-(1-(1-(3-((4-([1,1′-biphenyl]-3-yl)-5-chloropyrimidin-2-yl)amino)cyclohexane-1-carbonyl)piperidine-4- |
| | carbonyl)piperidin-4-yl)-1-oxoisoindolin-2-yl)piperidine-2,6-dione |
| 61 | 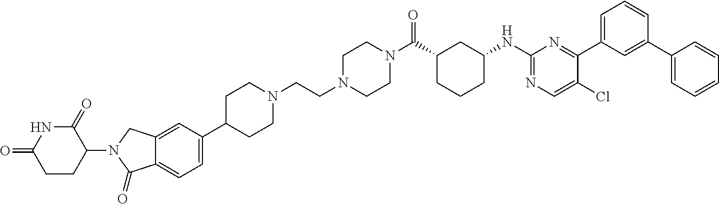 [SMILES: O=C1CCC(N2Cc3cc(C4CCN(CCN5CCN(C(=O)[C@H]6CCC[C@@H](Nc7ncc(Cl)c(-c8cccc(-c9ccccc9)c8)n7)C6)CC5)CC4)ccc3C2=O)C(=O)N1] |
| | cis-3-(5-(1-(2-(4-(3-((4-([1,1′-biphenyl]-3-yl)-5-chloropyrimidin-2-yl)amino)cyclohexane-1-carbonyl)piperazin-1-yl)- |
| | ethyl)piperidin-4-yl)-1-oxoisoindolin-2-yl)piperidine-2,6-dione |
| 62 | 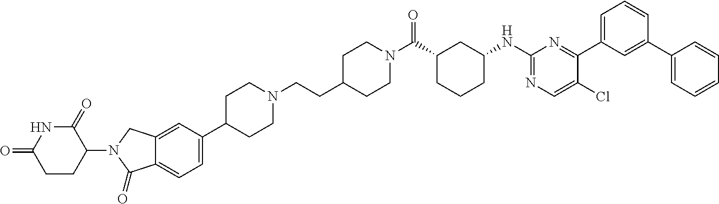 [SMILES: O=C1CCC(N2Cc3cc(C4CCN(CCC5CCN(C(=O)[C@H]6CCC[C@@H](Nc7ncc(Cl)c(-c8cccc(-c9ccccc9)c8)n7)C6)CC5)CC4)ccc3C2=O)C(=O)N1] |
| | cis-3-(5-(1-(2-(1-(3-((4-([1,1′-biphenyl]-3-yl)-5-chloropyrimidin-2-yl)amino)cyclohexane-1-carbonyl)piperidin-4- |
| | yl)ethyl)piperidin-4-yl)-1-oxoisoindolin-2-yl)piperidine-2,6-dione |
| 63 | 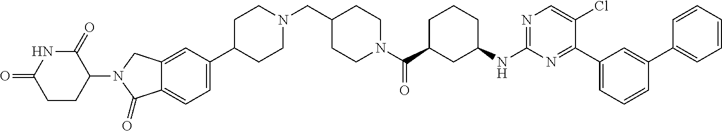 [SMILES: O=C1CCC(N2Cc3cc(C4CCN(CC5CCN(C(=O)[C@H]6CCC[C@@H](Nc7ncc(Cl)c(-c8cccc(-c9ccccc9)c8)n7)C6)CC5)CC4)ccc3C2=O)C(=O)N1] |
| | cis-3-(5-(1-((1-(3-((4-([1,1′-biphenyl]-3-yl)-5-chloropyrimidin-2-yl)amino)cyclohexane-1-carbonyl)piperidin-4-yl)- |
| | methyl)piperidin-4-yl)-1-oxoisoindolin-2-yl)piperidine-2,6-dione |
| 64 | 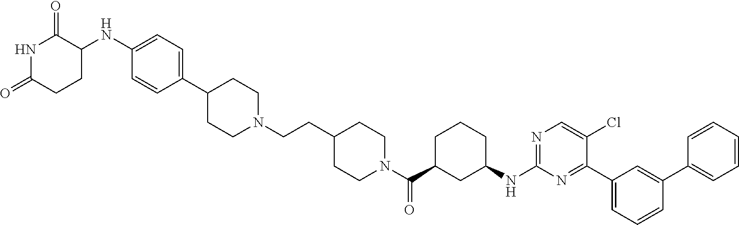 [SMILES: O=C1CCC(Nc2ccc(C3CCN(CCC4CCN(C(=O)[C@H]5CCC[C@@H](Nc6ncc(Cl)c(-c7cccc(-c8ccccc8)c7)n6)C5)CC4)CC3)cc2)C(=O)N1] |
| | cis-3-((4-(1-(2-(1-(3-((4-([1,1′-biphenyl]-3-yl)-5-chloropyrimidin-2-yl)amino)cyclohexane-1-carbonyl)piperidin-4- |
| | yl)ethyl)piperidin-4-yl)phenyl)amino)piperidine-2,6-dione |
| 65 | 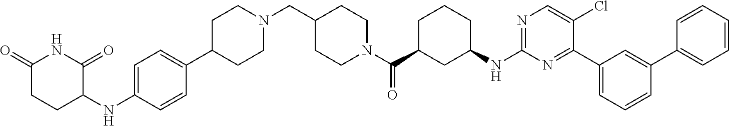 [SMILES: O=C1CCC(Nc2ccc(C3CCN(CC4CCN(C(=O)[C@H]5CCC[C@@H](Nc6ncc(Cl)c(-c7cccc(-c8ccccc8)c7)n6)C5)CC4)CC3)cc2)C(=O)N1] |
| | cis-3-((4-(1-((1-(3-((4-([1,1′-biphenyl]-3-yl)-5-chloropyrimidin-2-yl)amino)cyclohexane-1-carbonyl)piperidin-4-yl)- |
| | methyl)piperidin-4-yl)phenyl)amino)piperidine-2,6-dione |
| 66 | 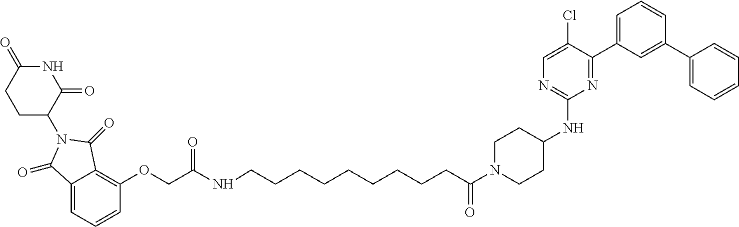 [SMILES: O=C(COc1cccc2c1C(=O)N(C1CCC(=O)NC1=O)C2=O)NCCCCCCCCCC(=O)N1CCC(Nc2ncc(Cl)c(-c3cccc(-c4ccccc4)c3)n2)CC1] |
| | N-(10-(4-((4-([1,1′-biphenyl]-3-yl)-5-chloropyrimidin-2-yl)amino)piperidin-1-yl)-10-oxodecyl)-2-((2-(2,6-dioxo- |
| | piperidin-3-yl)-1,3-dioxoisoindolin-4-yl)oxy)acetamide |
| 67 | 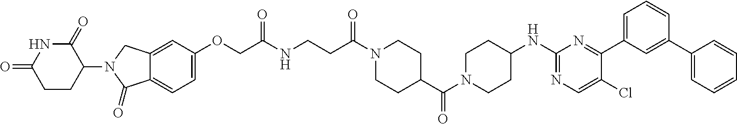 [SMILES: O=C(COc1ccc2c(c1)CN(C1CCC(=O)NC1=O)C2=O)NCCC(=O)N1CCC(C(=O)N2CCC(Nc3ncc(Cl)c(-c4cccc(-c5ccccc5)c4)n3)CC2)CC1] |
| | N-(3-(4-(4-((4-([1,1′-biphenyl]-3-yl)-5-chloropyrimidin-2-yl)amino)piperidine-1-carbonyl)piperidin-1-yl)-3-oxopropyl)- |
| | 2-((2-(2,6-dioxopiperidin-3-yl)-1-oxoisoindolin-5-yl)oxy)acetamide |
| 68 | 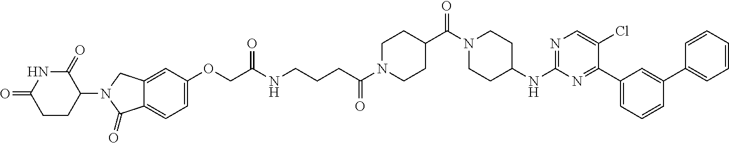 [SMILES: O=C(COc1ccc2c(c1)CN(C1CCC(=O)NC1=O)C2=O)NCCCC(=O)N1CCC(C(=O)N2CCC(Nc3ncc(Cl)c(-c4cccc(-c5ccccc5)c4)n3)CC2)CC1] |
| | N-(4-(4-(4-((4-([1,1′-biphenyl]-3-yl)-5-chloropyrimidin-2-yl)amino)piperidine-1-carbonyl)piperidin-1-yl)-4- |
| | oxobutyl)-2-((2-(2,6-dioxopiperidin-3-yl)-1-oxoisoindolin-5-yl)oxy)acetamide |
| 69 | 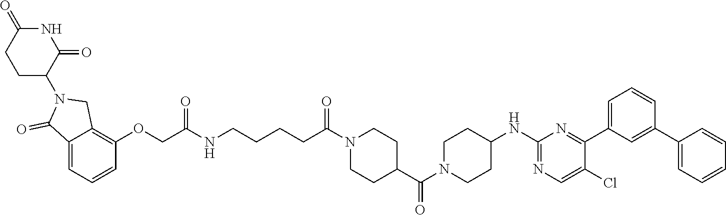 [SMILES: O=C(COc1cccc2c1CN(C1CCC(=O)NC1=O)C2=O)NCCCCC(=O)N1CCC(C(=O)N2CCC(Nc3ncc(Cl)c(-c4cccc(-c5ccccc5)c4)n3)CC2)CC1] |
| | N-(5-(4-(4-((4-([1,1′-biphenyl]-3-yl)-5-chloropyrimidin-2-yl)amino)piperidine-1-carbonyl)piperidin-1-yl)-5- |
| | oxopentyl)-2-((2-(2,6-dioxopiperidin-3-yl)-1-oxoisoindolin-4-yl)oxy)acetamide |
| 70 | 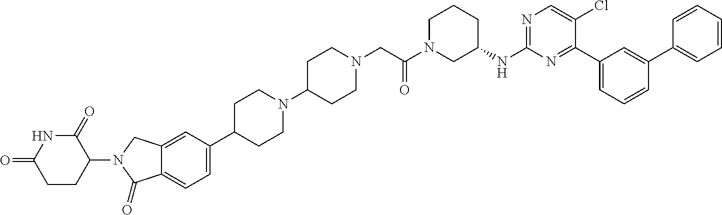 [SMILES: O=C1CCC(N2Cc3cc(C4CCN(C5CCN(CC(=O)N6CCC[C@H](Nc7ncc(Cl)c(-c8cccc(-c9ccccc9)c8)n7)C6)CC5)CC4)ccc3C2=O)C(=O)N1] |
| | 3-(5-(1′-(2-((S)-3-((4-([1,1′-biphenyl]-3-yl)-5-chloropyrimidin-2-yl)amino)piperidin-1-yl)-2-oxoethyl)-[1,4′- |
| | bipiperidin]-4-yl)-1-oxoisoindolin-2-yl)piperidine-2,6-dione |
| 71 | 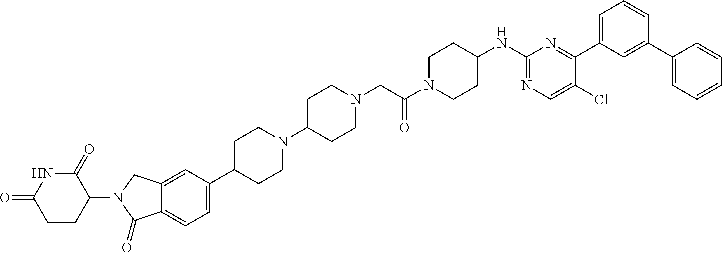 [SMILES: O=C1CCC(N2Cc3cc(C4CCN(C5CCN(CC(=O)N6CCC(Nc7ncc(Cl)c(-c8cccc(-c9ccccc9)c8)n7)CC6)CC5)CC4)ccc3C2=O)C(=O)N1] |
| | 3-(5-(1′-(2-(4-((4-([1,1′-biphenyl]-3-yl)-5-chloropyrimidin-2-yl)amino)piperidin-1-yl)-2-oxoethyl)-[1,4′-bipiperidin]- |
| | 4-yl)-1-oxoisoindolin-2-yl)piperidine-2,6-dione |
| 72 | 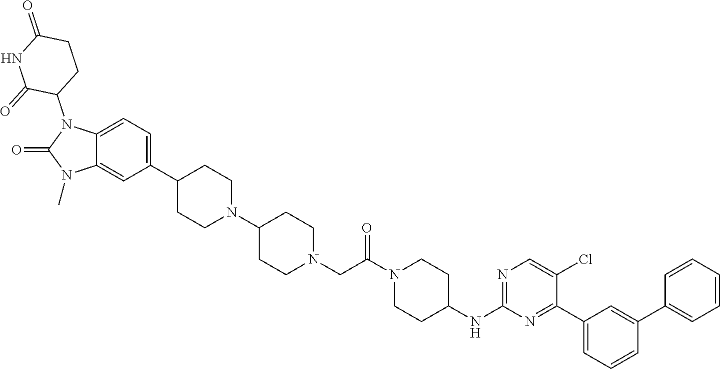 [SMILES: Cn1c(=O)n(C2CCC(=O)NC2=O)c2ccc(C3CCN(C4CCN(CC(=O)N5CCC(Nc6ncc(Cl)c(-c7cccc(-c8ccccc8)c7)n6)CC5)CC4)CC3)cc21] |
| | 3-(5-(1′-(2-(4-((4-([1,1′-biphenyl]-3-yl)-5-chloropyrimidin-2-yl)amino)piperidin-1-yl)-2-oxoethyl)-[1,4′-bipiperidin]- |
| | 4-yl)-3-methyl-2-oxo-2,3-dihydro-1H-benzo[d]imidazol-1-yl)piperidine-2,6-dione |
| 73 | 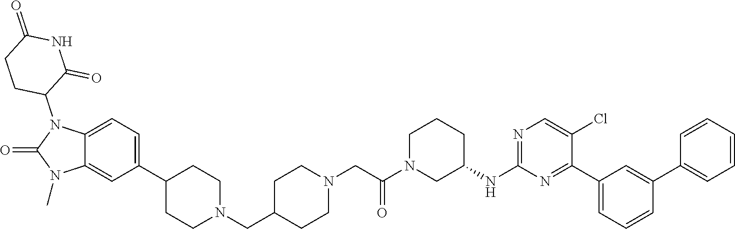 [SMILES: Cn1c(=O)n(C2CCC(=O)NC2=O)c2ccc(C3CCN(CC4CCN(CC(=O)N5CCC[C@H](Nc6ncc(Cl)c(-c7cccc(-c8ccccc8)c7)n6)C5)CC4)CC3)cc21] |
| | 3-(5-(1-((1-(2-((S)-3-((4-([1,1′-biphenyl]-3-yl)-5-chloropyrimidin-2-yl)amino)piperidin-1-yl)-2-oxoethyl)piperidin-4- |
| | yl)methyl)piperidin-4-yl)-3-methyl-2-oxo-2,3-dihydro-1H-benzo[d]imidazol-1-yl)piperidine-2,6-dione |
| 74 | 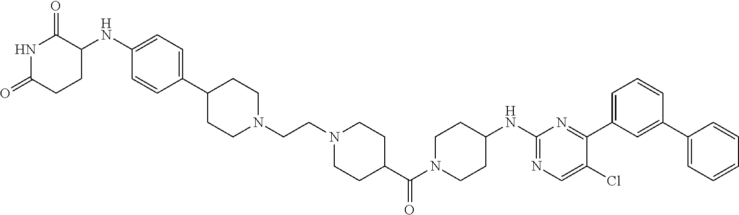 [SMILES: O=C1CCC(Nc2ccc(C3CCN(CCN4CCC(C(=O)N5CCC(Nc6ncc(Cl)c(-c7cccc(-c8ccccc8)c7)n6)CC5)CC4)CC3)cc2)C(=O)N1] |
| | 3-((4-(1-(2-(4-(4-((4-([1,1′-biphenyl]-3-yl)-5-chloropyrimidin-2-yl)amino)piperidine-1-carbonyl)piperidin-1-yl)ethyl)- |
| | piperidin-4-yl)phenyl)amino)piperidine-2,6-dione |
| 75 | 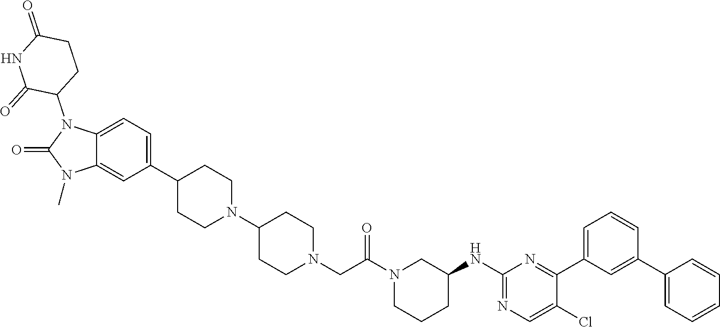 [SMILES: Cn1c(=O)n(C2CCC(=O)NC2=O)c2ccc(C3CCN(C4CCN(CC(=O)N5CCC[C@H](Nc6ncc(Cl)c(-c7cccc(-c8ccccc8)c7)n6)C5)CC4)CC3)cc21] |
| | 3-(5-(1′-(2-((S)-3-((4-([1,1′-biphenyl]-3-yl)-5-chloropyrimidin-2-yl)amino)piperidin-1-yl)-2-oxoethyl)-[1,4′- |
| | bipiperidin]-4-yl)-3-methyl-2-oxo-2,3-dihydro-1H-benzo[d]imidazol-1-yl)piperidine-2,6-dione |
| 76 | 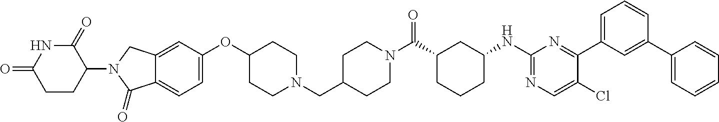 [SMILES: O=C1CCC(N2Cc3cc(OC4CCN(CC5CCN(C(=O)[C@H]6CCC[C@@H](Nc7ncc(Cl)c(-c8cccc(-c9ccccc9)c8)n7)C6)CC5)CC4)ccc3C2=O)C(=O)N1] |
| | cis-3-(5-((1-((1-(3-((4-([1,1′-biphenyl]-3-yl)-5-chloropyrimidin-2-yl)amino)cyclohexane-1-carbonyl)piperidin-4- |
| | yl)methyl)piperidin-4-yl)oxy)-1-oxoisoindolin-2-yl)piperidine-2,6-dione |
| 77 | 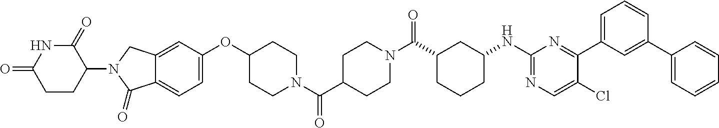 |
| | cis-3-(5-((1-(1-(3-((4-([1,1′-biphenyl]-3-yl)-5-chloropyrimidin-2-yl)amino)cyclohexane-1-carbonyl)piperidine-4- |
| | carbonyl)piperidin-4-yl)oxy)-1-oxoisoindolin-2-yl)piperidine-2,6-dione |
| 78 | 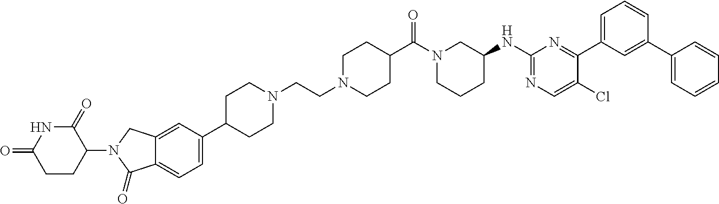 [SMILES: O=C1CCC(N2Cc3cc(C4CCN(CCN5CCC(C(=O)N6CCC[C@H](Nc7ncc(Cl)c(-c8cccc(-c9ccccc9)c8)n7)C6)CC5)CC4)ccc3C2=O)C(=O)N1] |
| | 3-(5-(1-(2-(4-((S)-3-((4-([1,1′-biphenyl]-3-yl)-5-chloropyrimidin-2-yl)amino)piperidine-1-carbonyl)piperidin-1-yl)- |
| | ethyl)piperidin-4-yl)-1-oxo-2,3-dihydro-1H-inden-2-yl)piperidine-2,6-dione |
| 79 | 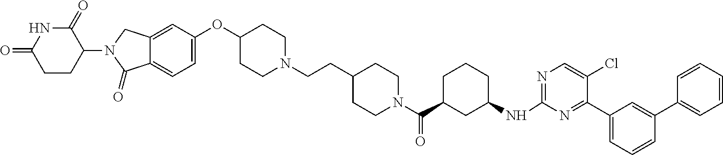 [SMILES: O=C1CCC(N2Cc3cc(OC4CCN(CCC5CCN(C(=O)[C@H]6CCC[C@@H](Nc7ncc(Cl)c(-c8cccc(-c9ccccc9)c8)n7)C6)CC5)CC4)ccc3C2=O)C(=O)N1] |
| | cis-3-(5-((1-(2-(1-(3-((4-([1,1′-biphenyl]-3-yl)-5-chloropyrimidin-2-yl)amino)cyclohexane-1-carbonyl)piperidin-4-yl)- |
| | ethyl)piperidin-4-yl)oxy)-1-oxoisoindolin-2-yl)piperidine-2,6-dione |
| 80 | 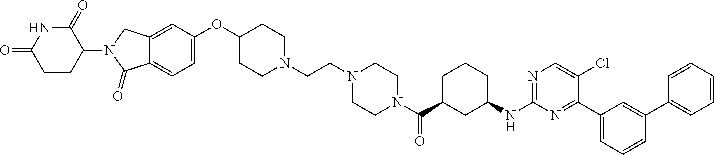 [SMILES: O=C1CCC(N2Cc3cc(OC4CCN(CCN5CCN(C(=O)[C@H]6CCC[C@@H](Nc7ncc(Cl)c(-c8cccc(-c9ccccc9)c8)n7)C6)CC5)CC4)ccc3C2=O)C(=O)N1] |
| | cis-3-(5-((1-(2-(4-(3-((4-([1,1′-biphenyl]-3-yl)-5-chloropyrimidin-2-yl)amino)cyclohexane-1-carbonyl)piperazin-1- |
| | yl)ethyl)piperidin-4-yl)oxy)-1-oxoisoindolin-2-yl)piperidine-2,6-dione |
| 81 | 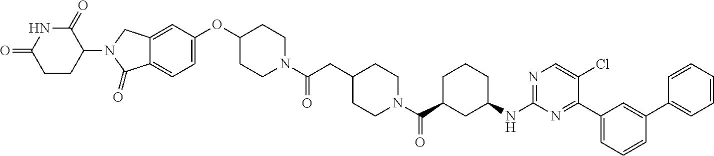 [SMILES: O=C1CCC(N2Cc3cc(OC4CCN(C(=O)CC5CCN(C(=O)[C@H]6CCC[C@@H](Nc7ncc(Cl)c(-c8cccc(-c9ccccc9)c8)n7)C6)CC5)CC4)ccc3C2=O)C(=O)N1] |
| | cis-3-(5-((1-(2-(1-(3-((4-([1,1′-biphenyl]-3-yl)-5-chloropyrimidin-2-yl)amino)cyclohexane-1-carbonyl)piperidin-4- |
| | yl)acetyl)piperidin-4-yl)oxy)-1-oxoisoindolin-2-yl)piperidine-2,6-dione |
| 82 | 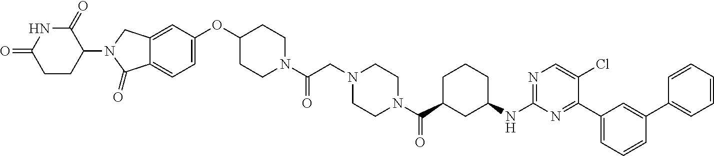 [SMILES: O=C1CCC(N2Cc3cc(OC4CCN(C(=O)CN5CCN(C(=O)[C@H]6CCC[C@@H](Nc7ncc(Cl)c(-c8cccc(-c9ccccc9)c8)n7)C6)CC5)CC4)ccc3C2=O)C(=O)N1] |
| | cis-3-(5-((1-(2-(4-(3-((4-([1,1′-biphenyl]-3-yl)-5-chloropyrimidin-2-yl)amino)cyclohexane-1-carbonyl)piperazin-1- |
| | yl)acetyl)piperidin-4-yl)oxy)-1-oxoisoindolin-2-yl)piperidine-2,6-dione |
| 83 | 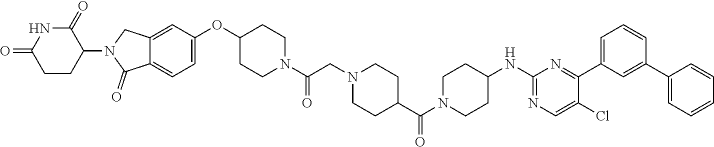 [SMILES: O=C1CCC(N2Cc3cc(OC4CCN(C(=O)CN5CCC(C(=O)N6CCC(Nc7ncc(Cl)c(-c8cccc(-c9ccccc9)c8)n7)CC6)CC5)CC4)ccc3C2=O)C(=O)N1] |
| | 3-(5-((1-(2-(4-(4-((4-([1,1′-biphenyl]-3-yl)-5-chloropyrimidin-2-yl)amino)piperidine-1-carbonyl)piperidin-1-yl)- |
| | acetyl)piperidin-4-yl)oxy)-1-oxoisoindolin-2-yl)piperidine-2,6-dione |
| 84 | 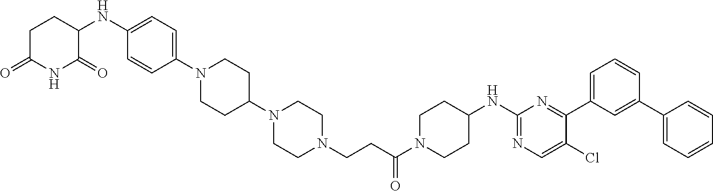 [SMILES: O=C1CCC(Nc2ccc(N3CCC(N4CCN(CCC(=O)N5CCC(Nc6ncc(Cl)c(-c7cccc(-c8ccccc8)c7)n6)CC5)CC4)CC3)cc2)C(=O)N1] |
| | 3-((4-(4-(4-(3-(4-((4-([1,1′-biphenyl]-3-yl)-5-chloropyrimidin-2-yl)amino)piperidin-1-yl)-3-oxopropyl)piperazin- |
| | 1-yl)piperidin-1-yl)phenyl)amino)piperidine-2,6-dione |
| 85 | 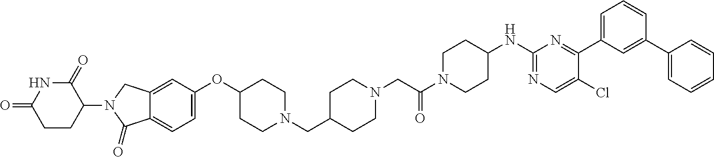 [SMILES: O=C1CCC(N2Cc3cc(OC4CCN(CC5CCN(CC(=O)N6CCC(Nc7ncc(Cl)c(-c8cccc(-c9ccccc9)c8)n7)CC6)CC5)CC4)ccc3C2=O)C(=O)N1] |
| | 3-(5-((1-((1-(2-(4-((4-([1,1′-biphenyl]-3-yl)-5-chloropyrimidin-2-yl)amino)piperidin-1-yl)-2-oxoethyl)piperidin-4- |
| | yl)methyl)piperidin-4-yl)oxy)-1-oxoisoindolin-2-yl)piperidine-2,6-dione |
| 86 | 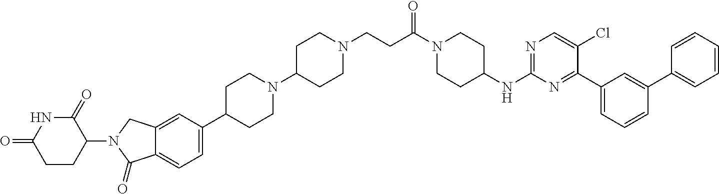 [SMILES: O=C1CCC(N2Cc3cc(C4CCN(C5CCN(CCC(=O)N6CCC(Nc7ncc(Cl)c(-c8cccc(-c9ccccc9)c8)n7)CC6)CC5)CC4)ccc3C2=O)C(=O)N1] |
| | 3-(5-(1′-(3-(4-((4-([1,1′-biphenyl]-3-yl)-5-chloropyrimidin-2-yl)amino)piperidin-1-yl)-3-oxopropyl)-[1,4′- |
| | bipiperidin]-4-yl)-1-oxoisoindolin-2-yl)piperidine-2,6-dione |
| 87 | 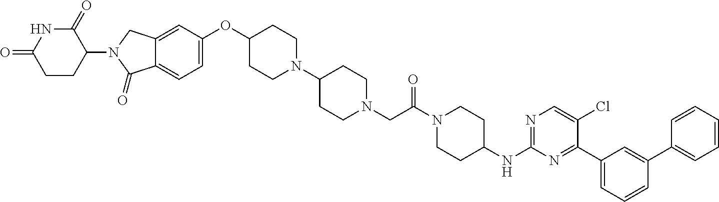 [SMILES: O=C1CCC(N2Cc3cc(OC4CCN(C5CCN(CC(=O)N6CCC(Nc7ncc(Cl)c(-c8cccc(-c9ccccc9)c8)n7)CC6)CC5)CC4)ccc3C2=O)C(=O)N1] |
| | 3-(5-((1′-(2-(4-((4-([1,1′-biphenyl]-3-yl)-5-chloropyrimidin-2-yl)amino)piperidin-1-yl)-2-oxoethyl)-[1,4′-bipiperidin]- |
| | 4-yl)oxy)-1-oxoisoindolin-2-yl)piperidine-2,6-dione |
| 88 | 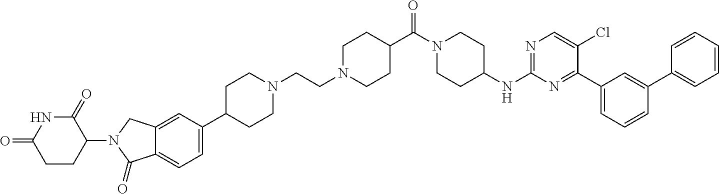 [SMILES: O=C1CCC(N2Cc3cc(C4CCN(CCN5CCC(C(=O)N6CCC(Nc7ncc(Cl)c(-c8cccc(-c9ccccc9)c8)n7)CC6)CC5)CC4)ccc3C2=O)C(=O)N1] |
| | 3-(5-(1-(2-(4-(4-((4-([1,1′-biphenyl]-3-yl)-5-chloropyrimidin-2-yl)amino)piperidine-1-carbonyl)piperidin-1-yl)ethyl)- |
| | piperidin-4-yl)-1-oxoisoindolin-2-yl)piperidine-2,6-dione |
| 89 | 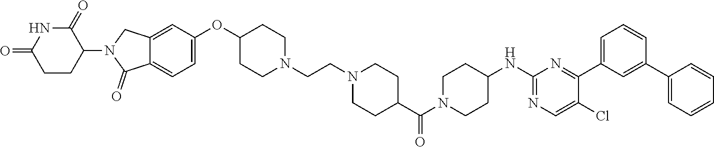 [SMILES: O=C1CCC(N2Cc3cc(OC4CCN(CCN5CCC(C(=O)N6CCC(Nc7ncc(Cl)c(-c8cccc(-c9ccccc9)c8)n7)CC6)CC5)CC4)ccc3C2=O)C(=O)N1] |
| | 3-(5-((1-(2-(4-(4-((4-([1,1′-biphenyl]-3-yl)-5-chloropyrimidin-2-yl)amino)piperidine-1-carbonyl)piperidin-1-yl) |
| | ethyl)piperidin-4-yl)oxy)-1-oxoisoindolin-2-yl)piperidine-2,6-dione |
| 90 | 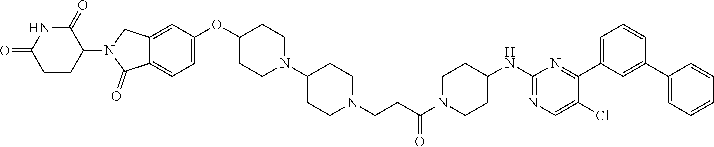 [SMILES: O=C1CCC(N2Cc3cc(OC4CCN(C5CCN(CCC(=O)N6CCC(Nc7ncc(Cl)c(-c8cccc(-c9ccccc9)c8)n7)CC6)CC5)CC4)ccc3C2=O)C(=O)N1] |
| | 3-(5-((1′-(3-(4-((4-([1,1′-biphenyl]-3-yl)-5-chloropyrimidin-2-yl)amino)piperidin-1-yl)-3-oxopropyl)-[1,4′-bipiperidin]- |
| | 4-yl)oxy)-1-oxoisoindolin-2-yl)piperidine-2,6-dione |
| 275 | 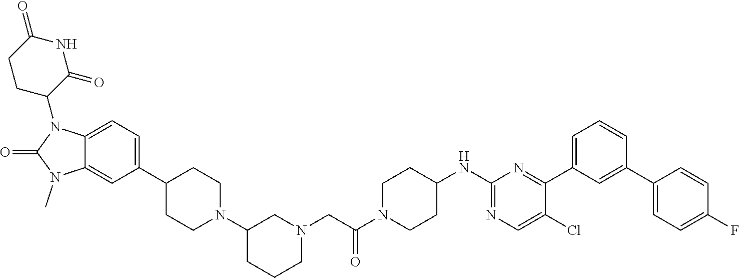 [SMILES: Cn1c(=O)n(C2CCC(=O)NC2=O)c2ccc(C3CCN(C4CCCN(CC(=O)N5CCC(Nc6ncc(Cl)c(-c7cccc(-c8ccc(F)cc8)c7)n6)CC5)C4)CC3)cc21] |
| | 3-(5-(1′-(2-(4-((5-chloro-4-(4′-fluoro-[1,1′-biphenyl]-3-yl)pyrimidin-2-yl)amino)piperidin-1-yl)-2-oxoethyl)-[1,3′- |
| | bipiperidin]-4-yl)-3-methyl-2-oxo-2,3-dihydro-1H-benzo[d]imidazol-1-yl)piperidine-2,6-dione |
| 276 | 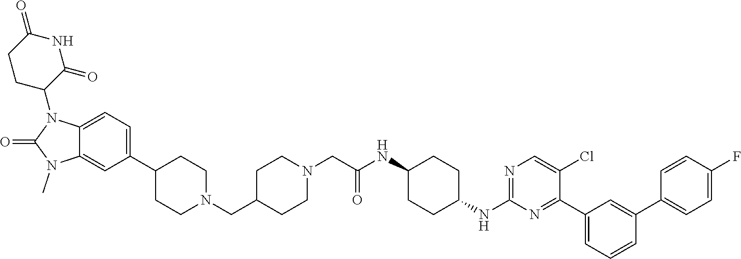 [SMILES: Cn1c(=O)n(C2CCC(=O)NC2=O)c2ccc(C3CCN(CC4CCN(CC(=O)N[C@H]5CC[C@H](Nc6ncc(Cl)c(-c7cccc(-c8ccc(F)cc8)c7)n6)CC5)CC4)CC3)cc21] |
| | trans-N-(4-((5-chloro-4-(4′-fluoro-[1,1′-biphenyl]-3-yl)pyrimidin-2-yl)amino)cyclohexyl)-2-(4-((4-(1-(2,6-dioxopiperidin- |
| | 3-yl)-3-methyl-2-oxo-2,3-dihydro-1H-benzo[d]imidazol-5-yl)piperidin-1-yl)methyl)piperidin-1-yl)acetamide |
| 277 | 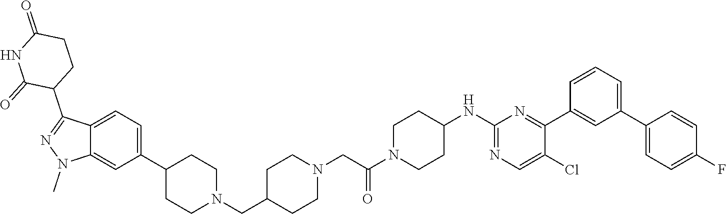 [SMILES: Cn1nc(C2CCC(=O)NC2=O)c2ccc(C3CCN(CC4CCN(CC(=O)N5CCC(Nc6ncc(Cl)c(-c7cccc(-c8ccc(F)cc8)c7)n6)CC5)CC4)CC3)cc21] |
| | 3-(6-(1-((1-(2-(4-((5-chloro-4-(4′-fluoro-[1,1′-biphenyl]-3-yl)pyrimidin-2-yl)amino)piperidin-1-yl)-2-oxoethyl) |
| | piperidin-4-yl)methyl)piperidin-4-yl)-1-methyl-1H-indazol-3-yl)piperidine-2,6-dione |
| 278 | 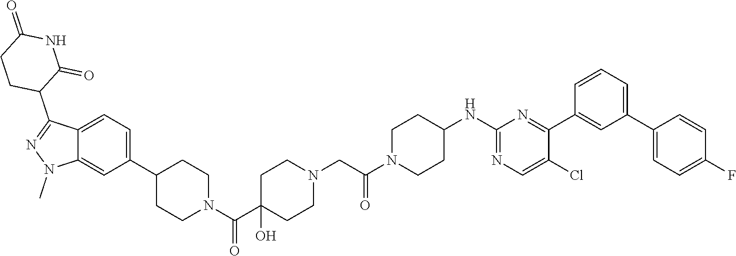 [SMILES: Cn1nc(C2CCC(=O)NC2=O)c2ccc(C3CCN(C(=O)C4(O)CCN(CC(=O)N5CCC(Nc6ncc(Cl)c(-c7cccc(-c8ccc(F)cc8)c7)n6)CC5)CC4)CC3)cc21] |
| | 3-(6-(1-(1-(2-(4-((5-chloro-4-(4′-fluoro-[1,1′-biphenyl]-3-yl)pyrimidin-2-yl)amino)piperidin-1-yl)-2-oxoethyl)-4- |
| | hydroxypiperidine-4-carbonyl)piperidin-4-yl)-1-methyl-1H-indazol-3-yl)piperidine-2,6-dione |
| 279 | 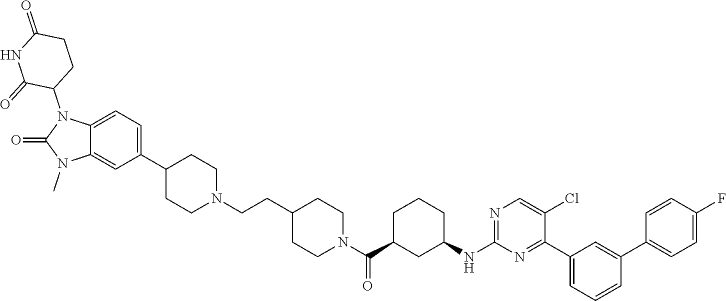 [SMILES: Cn1c(=O)n(C2CCC(=O)NC2=O)c2ccc(C3CCN(CCC4CCN(C(=O)[C@H]5CCC[C@@H](Nc6ncc(Cl)c(-c7cccc(-c8ccc(F)cc8)c7)n6)C5)CC4)CC3)cc21] |
| | rac-3-(5-(1-(2-(1-((1S,3R)-3-((5-chloro-4-(4′-fluoro-[1,1′-biphenyl]-3-yl)pyrimidin-2-yl)amino)cyclohexane-1-carbon- |
| | yl)piperidin-4-yl)ethyl)piperidin-4-yl)-3-methyl-2-oxo-2,3-dihydro-1H-benzo[d]imidazol-1-yl)piperidine-2,6-dione |
| 280 | 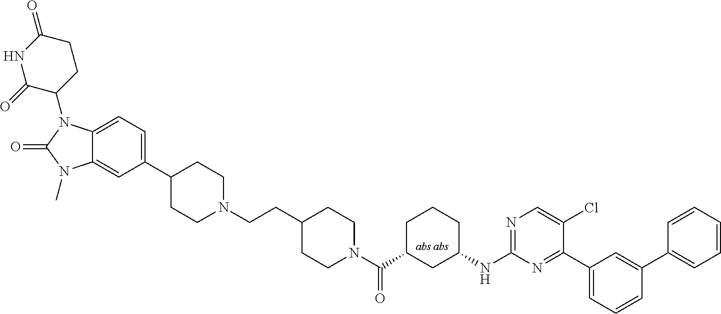 [SMILES: Cn1c(=O)n(C2CCC(=O)NC2=O)c2ccc(C3CCN(CCC4CCN(C(=O)[C@@H]5CCC[C@H](Nc6ncc(Cl)c(-c7cccc(-c8ccccc8)c7)n6)C5)CC4)CC3)cc21] |
| | 3-(5-(1-(2-(1-((1R,3S)-3-((4-([1,1′-biphenyl]-3-yl)-5-chloropyrimidin-2-yl)amino)cyclohexane-1-carbonyl)piperidin-4- |
| | yl)ethyl)piperidin-4-yl)-3-methyl-2-oxo-2,3-dihydro-1H-benzo[d]imidazol-1-yl)piperidine-2,6-dione |
| 281 | 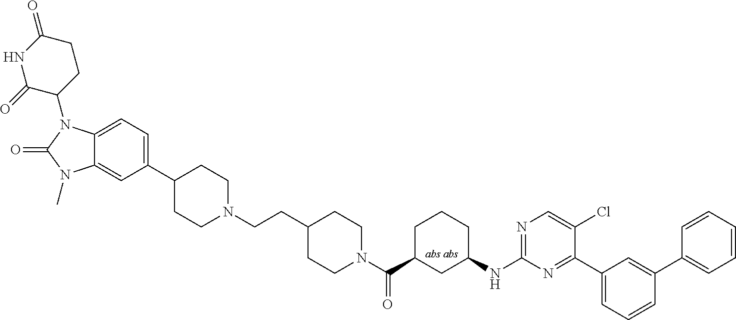 [SMILES: Cn1c(=O)n(C2CCC(=O)NC2=O)c2ccc(C3CCN(CCC4CCN(C(=O)[C@H]5CCC[C@@H](Nc6ncc(Cl)c(-c7cccc(-c8ccccc8)c7)n6)C5)CC4)CC3)cc21] |
| | 3-(5-(1-(2-(1-((1S,3R)-3-((4-([1,1′-biphenyl]-3-yl)-5-chloropyrimidin-2-yl)amino)cyclohexane-1-carbonyl)piperidin-4- |
| | yl)ethyl)piperidin-4-yl)-3-methyl-2-oxo-2,3-dihydro-1H-benzo[d]imidazol-1-yl)piperidine-2,6-dione |
| 282 | 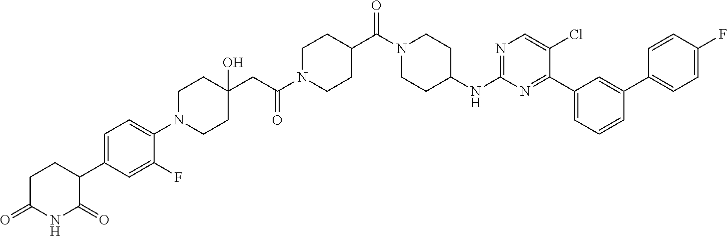 [SMILES: O=C1CCC(c2ccc(N3CCC(O)(CC(=O)N4CCC(C(=O)N5CCC(Nc6ncc(Cl)c(-c7cccc(-c8ccc(F)cc8)c7)n6)CC5)CC4)CC3)c(F)c2)C(=O)N1] |
| | 3-(4-(4-(2-(4-(4-((5-chloro-4-(4′-fluoro-[1,1′-biphenyl]-3-yl)pyrimidin-2-yl)amino)piperidine-1-carbonyl)piperidin- |
| | 1-yl)-2-oxoethyl)-4-hydroxypiperidin-1-yl)-3-fluorophenyl)piperidine-2,6-dione |
| 283 | 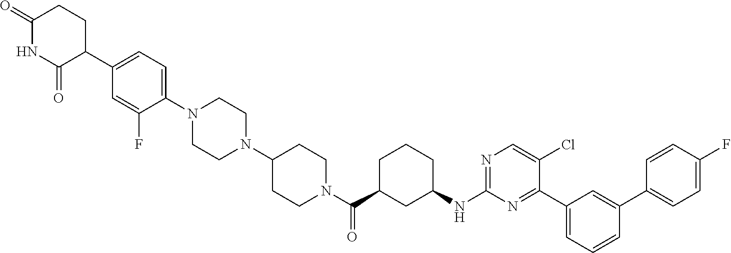 [SMILES: O=C1CCC(c2ccc(N3CCN(C4CCN(C(=O)[C@H]5CCC[C@@H](Nc6ncc(Cl)c(-c7cccc(-c8ccc(F)cc8)c7)n6)C5)CC4)CC3)c(F)c2)C(=O)N1] |
| | rac-3-(4-(4-(1-((1S,3R)-3-((5-chloro-4-(4′-fluoro-[1,1′-biphenyl]-3-yl)pyrimidin-2-yl)amino)cyclohexane-1-carbonyl)- |
| | piperidin-4-yl)piperazin-1-yl)-3-fluorophenyl)piperidine-2,6-dione |
| 284 | 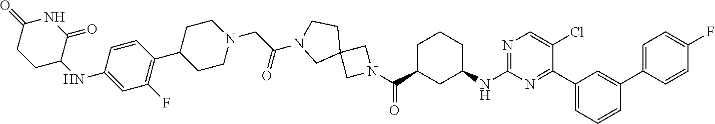 [SMILES: O=C1CCC(Nc2ccc(C3CCN(CC(=O)N4CCC5(C4)CN(C(=O)[C@H]4CCC[C@@H](Nc6ncc(Cl)c(-c7cccc(-c8ccc(F)cc8)c7)n6)C4)C5)CC3)c(F)c2)C(=O)N1] |
| | cis-3-((4-(1-(2-(2-(3-((5-chloro-4-(4′-fluoro-[1,1′-biphenyl]-3-yl)pyrimidin-2-yl)amino)cyclohexane-1-carbonyl)-2,6- |
| | diazaspiro[3.4]octan-6-yl)-2-oxoethyl)piperidin-4-yl)-3-fluorophenyl)amino)piperidine-2,6-dione |
| 285 | 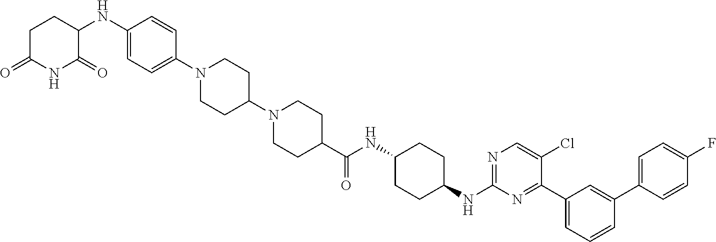 [SMILES: O=C1CCC(Nc2ccc(N3CCC(N4CCC(C(=O)N[C@H]5CC[C@H](Nc6ncc(Cl)c(-c7cccc(-c8ccc(F)cc8)c7)n6)CC5)CC4)CC3)cc2)C(=O)N1] |
| | trans-N-(4-((5-chloro-4-(4′-fluoro-[1,1′-biphenyl]-3-yl)pyrimidin-2-yl)amino)cyclohexyl)-1′-(4-((2,6-dioxopiperidin- |
| | 3-yl)amino)phenyl)-[1,4′-bipiperidine]-4-carboxamide |
| 286 | 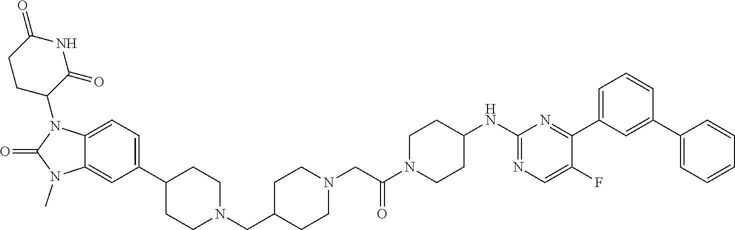 [SMILES: Cn1c(=O)n(C2CCC(=O)NC2=O)c2ccc(C3CCN(CC4CCN(CC(=O)N5CCC(Nc6ncc(F)c(-c7cccc(-c8ccccc8)c7)n6)CC5)CC4)CC3)cc21] |
| | 3-(5-(1-((1-(2-(4-((4-([1,1′-biphenyl]-3-yl)-5-fluoropyrimidin-2-yl)amino)piperidin-1-yl)-2-oxoethyl)piperidin-4-yl)- |
| | methyl)piperidin-4-yl)-3-methyl-2-oxo-2,3-dihydro-1H-benzo[d]imidazol-1-yl)piperidine-2,6-dione |
| 287 | 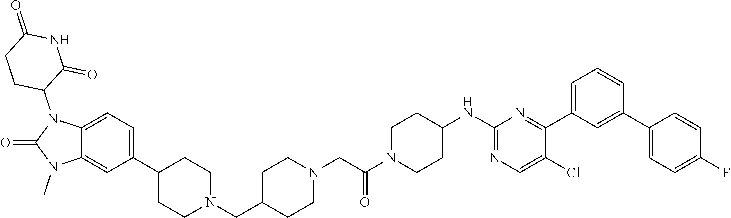 [SMILES: Cn1c(=O)n(C2CCC(=O)NC2=O)c2ccc(C3CCN(CC4CCN(CC(=O)N5CCC(Nc6ncc(Cl)c(-c7cccc(-c8ccc(F)cc8)c7)n6)CC5)CC4)CC3)cc21] |
| | 3-(5-(1-((1-(2-(4-((5-chloro-4-(4′-fluoro-[1,1′-biphenyl]-3-yl)pyrimidin-2-yl)amino)piperidin-1-yl)-2-oxoethyl)piperidin- |
| | 4-yl)methyl)piperidin-4-yl)-3-methyl-2-oxo-2,3-dihydro-1H-benzo[d]imidazol-1-yl)piperidine-2,6-dione |
| 288 | 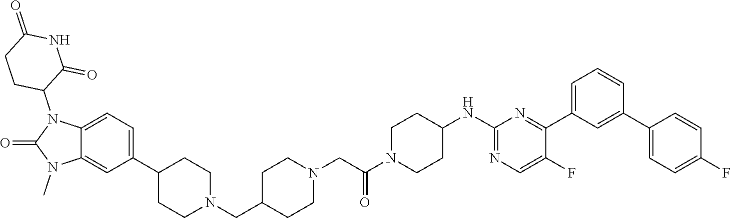 [SMILES: Cn1c(=O)n(C2CCC(=O)NC2=O)c2ccc(C3CCN(CC4CCN(CC(=O)N5CCC(Nc6ncc(F)c(-c7cccc(-c8ccc(F)cc8)c7)n6)CC5)CC4)CC3)cc21] |
| | 3-(5-(1-((1-(2-(4-((5-fluoro-4-(4′-fluoro-[1,1′-biphenyl]-3-yl)pyrimidin-2-yl)amino)piperidin-1-yl)-2-oxoethyl)piperidin- |
| | 4-yl)methyl)piperidin-4-yl)-3-methyl-2-oxo-2,3-dihydro-1H-benzo[d]imidazol-1-yl)piperidine-2,6-dione |
| 289 | 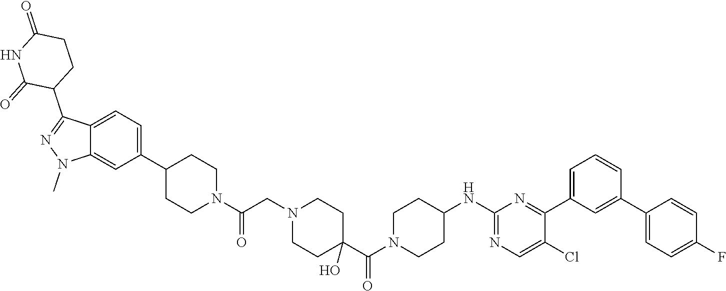 [SMILES: Cn1nc(C2CCC(=O)NC2=O)c2ccc(C3CCN(C(=O)CN4CCC(O)(C(=O)N5CCC(Nc6ncc(Cl)c(-c7cccc(-c8ccc(F)cc8)c7)n6)CC5)CC4)CC3)cc21] |
| | 3-(6-(1-(2-(4-(4-((5-chloro-4-(4′-fluoro-[1,1′-biphenyl]-3-yl)pyrimidin-2-yl)amino)piperidine-1-carbonyl)-4-hydroxy- |
| | piperidin-1-yl)acetyl)piperidin-4-yl)-1-methyl-1H-indazol-3-yl)piperidine-2,6-dione |
| 290 | 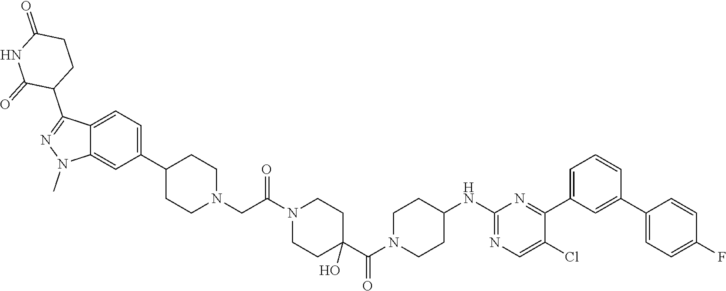 [SMILES: Cn1nc(C2CCC(=O)NC2=O)c2ccc(C3CCN(CC(=O)N4CCC(O)(C(=O)N5CCC(Nc6ncc(Cl)c(-c7cccc(-c8ccc(F)cc8)c7)n6)CC5)CC4)CC3)cc21] |
| | 3-(6-(1-(2-(4-(4-((5-chloro-4-(4′-fluoro-[1,1′-biphenyl]-3-yl)pyrimidin-2-yl)amino)piperidine-1-carbonyl)-4-hydroxy- |
| | piperidin-1-yl)-2-oxoethyl)piperidin-4-yl)-1-methyl-1H-indazol-3-yl)piperidine-2,6-dione |
| 291 | 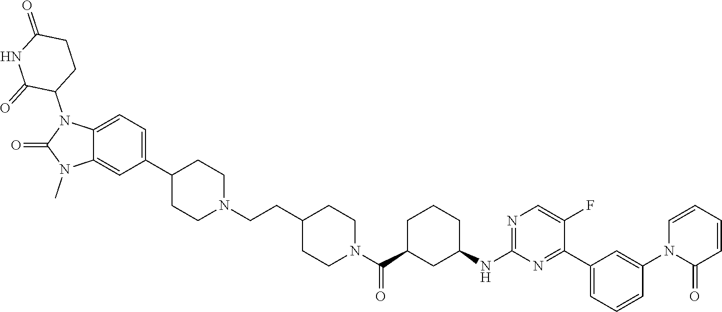 [SMILES: Cn1c(=O)n(C2CCC(=O)NC2=O)c2ccc(C3CCN(CCC4CCN(C(=O)[C@H]5CCC[C@@H](Nc6ncc(F)c(-c7cccc(-n8ccccc8=O)c7)n6)C5)CC4)CC3)cc21] |
| | rac-3-(5-(1-(2-(1-((1S,3R)-3-((5-fluoro-4-(3-(2-oxopyridin-1(2H)-yl)phenyl)pyrimidin-2-yl)amino)cyclohexane-1- |
| | carbonyl)piperidin-4-yl)ethyl)piperidin-4-yl)-3-methyl-2-oxo-2,3-dihydro-1H-benzo[d]imidazol-1-yl)piperidine- |
| | 2,6-dione |
| 292 | 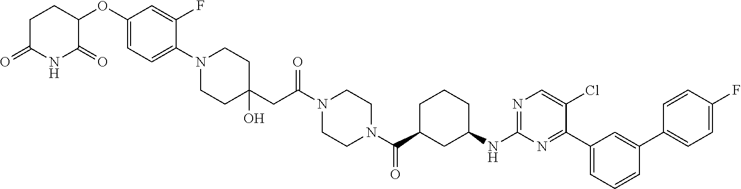 [SMILES: O=C1CCC(Oc2ccc(N3CCC(O)(CC(=O)N4CCN(C(=O)[C@H]5CCC[C@@H](Nc6ncc(Cl)c(-c7cccc(-c8ccc(F)cc8)c7)n6)C5)CC4)CC3)c(F)c2)C(=O)N1] |
| | rac-3-(4-(4-(2-(4-((1S,3R)-3-((5-chloro-4-(4′-fluoro-[1,1′-biphenyl]-3-yl)pyrimidin-2-yl)amino)cyclohexane-1- |
| | carbonyl)piperazin-1-yl)-2-oxoethyl)-4-hydroxypiperidin-1-yl)-3-fluorophenoxy)piperidine-2,6-dione |
| 293 | 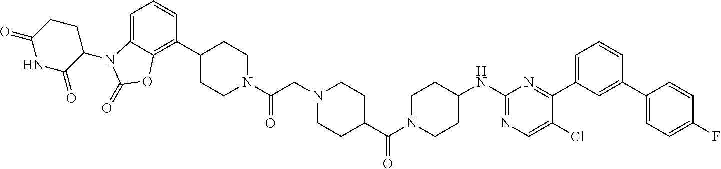 [SMILES: O=C1CCC(n2c(=O)oc3c(C4CCN(C(=O)CN5CCC(C(=O)N6CCC(Nc7ncc(Cl)c(-c8cccc(-c9ccc(F)cc9)c8)n7)CC6)CC5)CC4)cccc32)C(=O)N1] |
| | 3-(7-(1-(2-(4-(4-((5-chloro-4-(4′-fluoro-[1,1′-biphenyl]-3-yl)pyrimidin-2-yl)amino)piperidine-1-carbonyl)piperidin- |
| | 1-yl)acetyl)piperidin-4-yl)-2-oxobenzo[d]oxazol-3(2H)-yl)piperidine-2,6-dione |
| 294 | 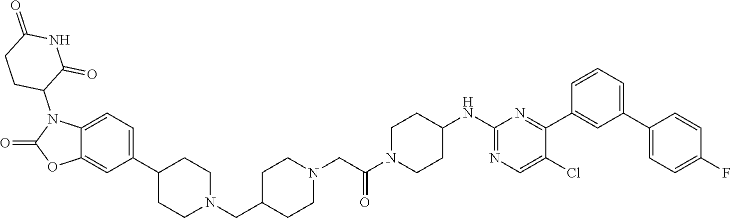 [SMILES: O=C1CCC(n2c(=O)oc3cc(C4CCN(CC5CCN(CC(=O)N6CCC(Nc7ncc(Cl)c(-c8cccc(-c9ccc(F)cc9)c8)n7)CC6)CC5)CC4)ccc32)C(=O)N1] |
| | 3-(6-(1-((1-(2-(4-((5-chloro-4-(4′-fluoro-[1,1′-biphenyl]-3-yl)pyrimidin-2-yl)amino)piperidin-1-yl)-2-oxoethyl)piperidin- |
| | 4-yl)methyl)piperidin-4-yl)-2-oxobenzo[d]oxazol-3(2H)-yl)piperidine-2,6-dione |
| 295 | 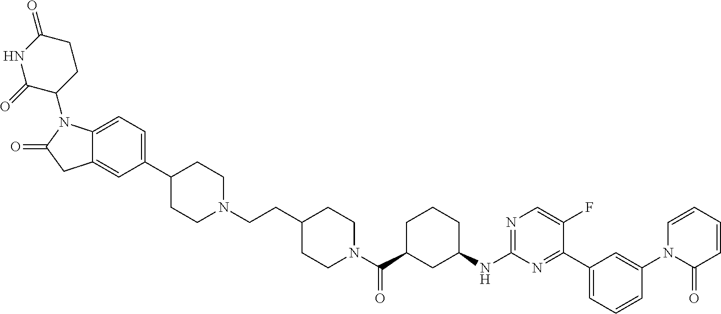 [SMILES: O=C1CCC(N2C(=O)Cc3cc(C4CCN(CCC5CCN(C(=O)[C@H]6CCC[C@@H](Nc7ncc(F)c(-c8cccc(-n9ccccc9=O)c8)n7)C6)CC5)CC4)ccc32)C(=O)N1] |
| | rac-3-(6-(1-(2-(1-((1S,3R)-3-((5-fluoro-4-(3-(2-oxopyridin-1(2H)-yl)phenyl)pyrimidin-2-yl)amino)cyclohexane-1- |
| | carbonyl)piperidin-4-yl)ethyl)piperidin-4-yl)-2-oxobenzo[d]oxazol-3(2H)-yl)piperidine-2,6-dione |
| 296 | 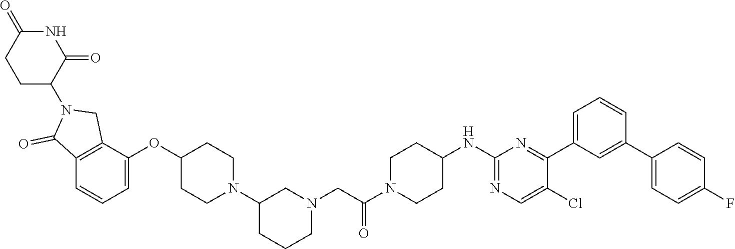 [SMILES: O=C1CCC(N2Cc3c(OC4CCN(C5CCCN(CC(=O)N6CCC(Nc7ncc(Cl)c(-c8cccc(-c9ccc(F)cc9)c8)n7)CC6)C5)CC4)cccc3C2=O)C(=O)N1] |
| | 3-(4-((1′-(2-(4-((5-chloro-4-(4′-fluoro-[1,1′-biphenyl]-3-yl)pyrimidin-2-yl)amino)piperidin-1-yl)-2-oxoethyl)-[1,3′- |
| | bipiperidin]-4-yl)oxy)-1-oxoisoindolin-2-yl)piperidine-2,6-dione |
| 297 | 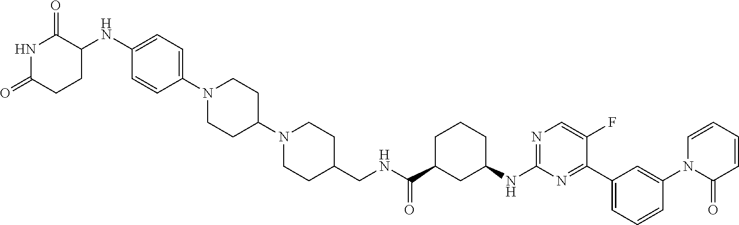 [SMILES: O=C1CCC(Nc2ccc(N3CCC(N4CCC(CNC(=O)[C@H]5CCC[C@@H](Nc6ncc(F)c(-c7cccc(-n8ccccc8=O)c7)n6)C5)CC4)CC3)cc2)C(=O)N1] |
| | rac-(1S,3R)-N-((1′-(4-((2,6-dioxopiperidin-3-yl)amino)phenyl)-[1,4′-bipiperidin]-4-yl)methyl)-3-((5-fluoro-4-(3-(2- |
| | oxopyridin-1(2H)-yl)phenyl)pyrimidin-2-yl)amino)cyclohexane-1-carboxamide |
| 298 | 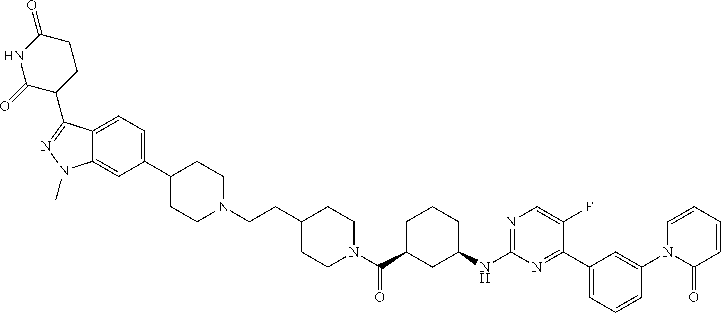 [SMILES: Cn1nc(C2CCC(=O)NC2=O)c2ccc(C3CCN(CCC4CCN(C(=O)[C@H]5CCC[C@@H](Nc6ncc(F)c(-c7cccc(-n8ccccc8=O)c7)n6)C5)CC4)CC3)cc21] |
| | 3-(6-(1-(2-(1-(cis-3-((5-fluoro-4-(3-(2-oxopyridin-1(2H)-yl)phenyl)pyrimidin-2-yl)amino)cyclohexane-1-carbonyl) |
| | piperidin-4-yl)ethyl)piperidin-4-yl)-1-methyl-1H-indazol-3-yl)piperidine-2,6-dione |
| 299 | 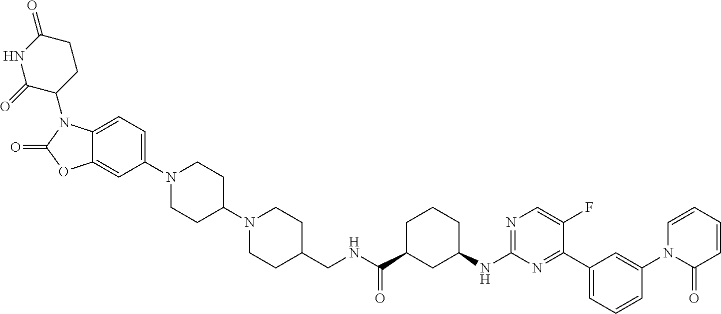 [SMILES: O=C1CCC(n2c(=O)oc3cc(N4CCC(N5CCC(CNC(=O)[C@H]6CCC[C@@H](Nc7ncc(F)c(-c8cccc(-n9ccccc9=O)c8)n7)C6)CC5)CC4)ccc32)C(=O)N1] |
| | rac-(1S,3R)-N-((1′-(3-(2,6-dioxopiperidin-3-yl)-2-oxo-2,3-dihydrobenzo[d]oxazol-6-yl)-[1,4′-bipiperidin]-4-yl)- |
| | methyl)-3-((5-fluoro-4-(3-(2-oxopyridin-1(2H)-yl)phenyl)pyrimidin-2-yl)amino)cyclohexane-1-carboxamide |
| 300 | 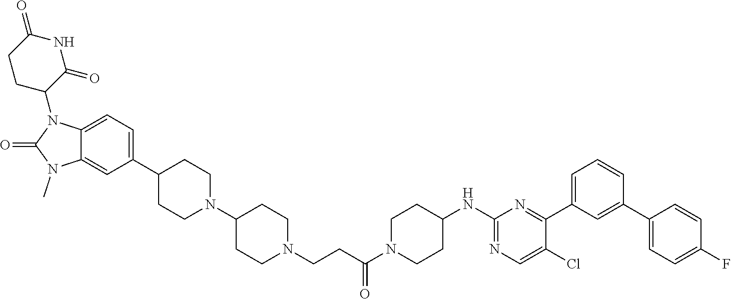 [SMILES: Cn1c(=O)n(C2CCC(=O)NC2=O)c2ccc(C3CCN(C4CCN(CCC(=O)N5CCC(Nc6ncc(Cl)c(-c7cccc(-c8ccc(F)cc8)c7)n6)CC5)CC4)CC3)cc21] |
| | 3-(5-(1′-(3-(4-((5-chloro-4-(4′-fluoro-[1,1′-biphenyl]-3-yl)pyrimidin-2-yl)amino)piperidin-1-yl)-3-oxopropyl)-[1,4′- |
| | bipiperidin]-4-yl)-3-methyl-2-oxo-2,3-dihydro-1H-benzo[d]imidazol-1-yl)piperidine-2,6-dione |
| 301 | 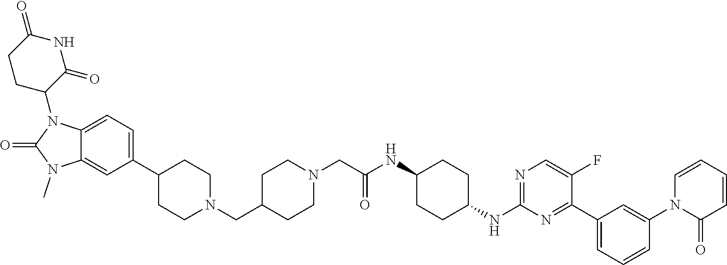 [SMILES: Cn1c(=O)n(C2CCC(=O)NC2=O)c2ccc(C3CCN(CC4CCN(CC(=O)N[C@H]5CC[C@H](Nc6ncc(F)c(-c7cccc(-n8ccccc8=O)c7)n6)CC5)CC4)CC3)cc21] |
| | trans-2-(4-((4-(1-(2,6-dioxopiperidin-3-yl)-3-methyl-2-oxo-2,3-dihydro-1H-benzo[d]imidazol-5-yl)piperidin-1-yl)meth- |
| | yl)piperidin-1-yl)-N-(4-((5-fluoro-4-(3-(2-oxopyridin-1(2H)-yl)phenyl)pyrimidin-2-yl)amino)cyclohexyl)acetamide |
| 302 | 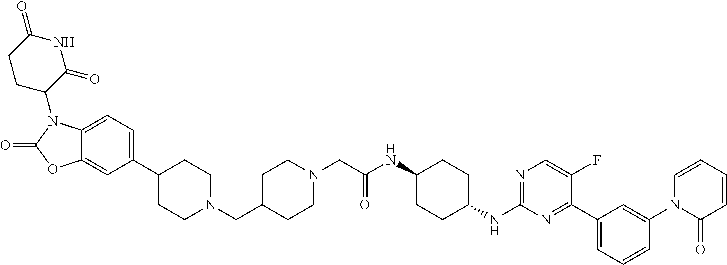 [SMILES: O=C1CCC(n2c(=O)oc3cc(C4CCN(CC5CCN(CC(=O)N[C@H]6CC[C@H](Nc7ncc(F)c(-c8cccc(-n9ccccc9=O)c8)n7)CC6)CC5)CC4)ccc32)C(=O)N1] |
| | trans-2-(4-((4-(3-(2,6-dioxopiperidin-3-yl)-2-oxo-2,3-dihydrobenzo[d]oxazol-6-yl)piperidin-1-yl)methyl)piperidin-1- |
| | yl)-N-(4-((5-fluoro-4-(3-(2-oxopyridin-1(2H)-yl)phenyl)pyrimidin-2-yl)amino)cyclohexyl)acetamide |
| 303 | 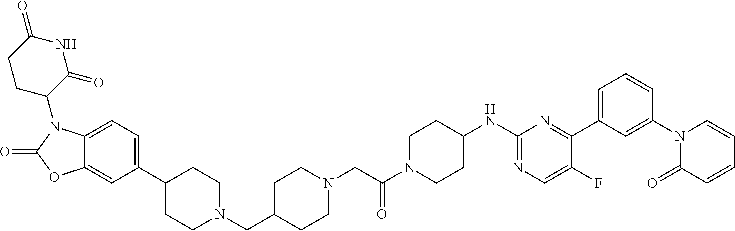 [SMILES: O=C1CCC(n2c(=O)oc3cc(C4CCN(CC5CCN(CC(=O)N6CCC(Nc7ncc(F)c(-c8cccc(-n9ccccc9=O)c8)n7)CC6)CC5)CC4)ccc32)C(=O)N1] |
| | 3-(6-(1-((1-(2-(4-((5-fluoro-4-(3-(2-oxopyridin-1(2H)-yl)phenyl)pyrimidin-2-yl)amino)piperidin-1-yl)-2-oxoethyl)- |
| | piperidin-4-yl)methyl)piperidin-4-yl)-2-oxobenzo[d]oxazol-3(2H)-yl)piperidine-2,6-dione |
| 304 | 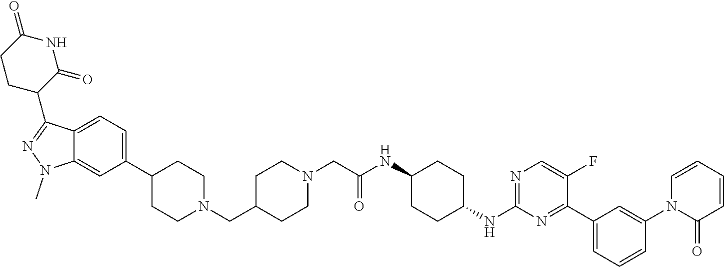 [SMILES: Cn1nc(C2CCC(=O)NC2=O)c2ccc(C3CCN(CC4CCN(CC(=O)N[C@H]5CC[C@H](Nc6ncc(F)c(-c7cccc(-n8ccccc8=O)c7)n6)CC5)CC4)CC3)cc21] |
| | trans-2-(4-((4-(3-(2,6-dioxopiperidin-3-yl)-1-methyl-1H-indazol-6-yl)piperidin-1-yl)methyl)piperidin-1-yl)-N-(4-((5- |
| | fluoro-4-(3-(2-oxopyridin-1(2H)-yl)phenyl)pyrimidin-2-yl)amino)cyclohexyl)acetamide |
| 305 | 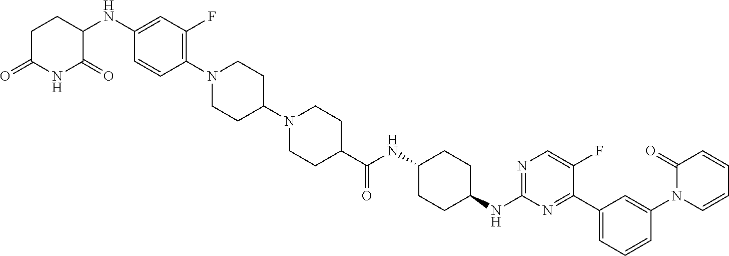 [SMILES: O=C1CCC(Nc2ccc(N3CCC(N4CCC(C(=O)N[C@H]5CC[C@H](Nc6ncc(F)c(-c7cccc(-n8ccccc8=O)c7)n6)CC5)CC4)CC3)c(F)c2)C(=O)N1] |
| | trans-1′-(4-((2,6-dioxopiperidin-3-yl)amino)-2-fluorophenyl)-N-(4-((5-fluoro-4-(3-(2-oxopyridin-1(2H)-yl)phenyl)- |
| | pyrimidin-2-yl)amino)cyclohexyl)-[1,4′-bipiperidine]-4-carboxamide |
| 306 | 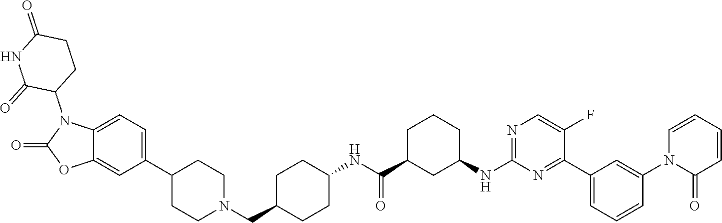 [SMILES: O=C1CCC(n2c(=O)oc3cc(C4CCN(C[C@H]5CC[C@H](NC(=O)[C@H]6CCC[C@@H](Nc7ncc(F)c(-c8cccc(-n9ccccc9=O)c8)n7)C6)CC5)CC4)ccc32)C(=O)N1] |
| | cis-trans-N-(4-((4-(3-(2,6-dioxopiperidin-3-yl)-2-oxo-2,3-dihydrobenzo[d]oxazol-6-yl)piperidin-1-yl)methyl)- |
| | cyclohexyl)-3-((5-fluoro-4-(3-(2-oxopyridin-1(2H)-yl)phenyl)pyrimidin-2-yl)amino)cyclohexane-1-carboxamide |
| 307 | 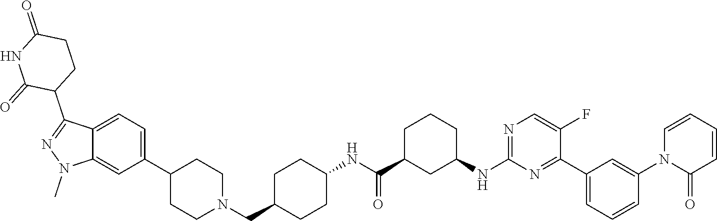 [SMILES: Cn1nc(C2CCC(=O)NC2=O)c2ccc(C3CCN(C[C@H]4CC[C@H](NC(=O)[C@H]5CCC[C@@H](Nc6ncc(F)c(-c7cccc(-n8ccccc8=O)c7)n6)C5)CC4)CC3)cc21] |
| | cis-trans-N-(4-((4-(3-(2,6-dioxopiperidin-3-yl)-1-methyl-1H-indazol-6-yl)piperidin-1-yl)methyl)cyclohexyl)-3-((5- |
| | fluoro-4-(3-(2-oxopyridin-1(2H)-yl)phenyl)pyrimidin-2-yl)amino)cyclohexane-1-carboxamide |
| 308 | 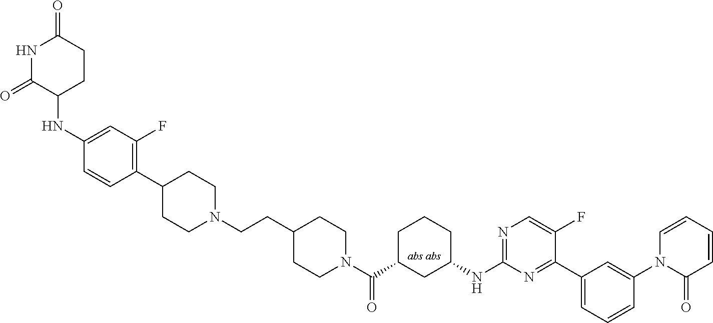 [SMILES: O=C1CCC(Nc2ccc(C3CCN(CCC4CCN(C(=O)[C@@H]5CCC[C@H](Nc6ncc(F)c(-c7cccc(-n8ccccc8=O)c7)n6)C5)CC4)CC3)c(F)c2)C(=O)N1] |
| | 3-((3-fluoro-4-(1-(2-(1-((1R,3S)-3-((5-fluoro-4-(3-(2-oxopyridin-1(2H)-yl)phenyl)pyrimidin-2-yl)amino)cyclo- |
| | hexane-1-carbonyl)piperidin-4-yl)ethyl)piperidin-4-yl)phenyl)amino)piperidine-2,6-dione |
| 309 | 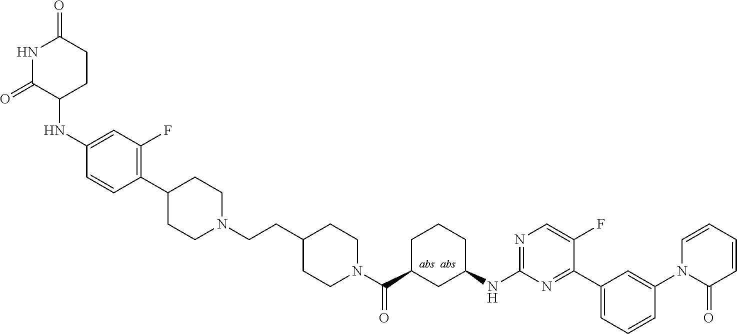 [SMILES: O=C1CCC(Nc2ccc(C3CCN(CCC4CCN(C(=O)[C@H]5CCC[C@@H](Nc6ncc(F)c(-c7cccc(-n8ccccc8=O)c7)n6)C5)CC4)CC3)c(F)c2)C(=O)N1] |
| | 3-((3-fluoro-4-(1-(2-(1-((1S,3R)-3-((5-fluoro-4-(3-(2-oxopyridin-1(2H)-yl)phenyl)pyrimidin-2-yl)amino)cyclohexane- |
| | 1-carbonyl)piperidin-4-yl)ethyl)piperidin-4-yl)phenyl)amino)piperidine-2,6-dione |
| 310 | 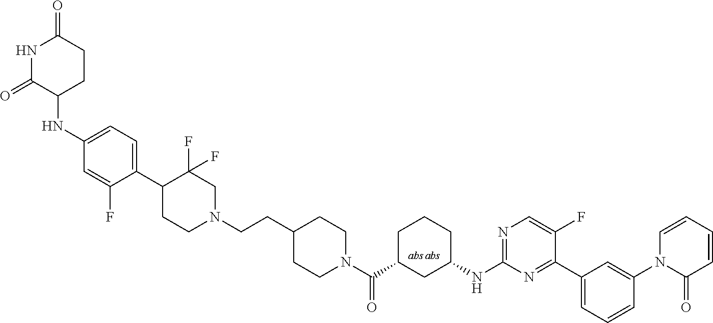 [SMILES: O=C1CCC(Nc2ccc(C3CCN(CCC4CCN(C(=O)[C@@H]5CCC[C@H](Nc6ncc(F)c(-c7cccc(-n8ccccc8=O)c7)n6)C5)CC4)CC3(F)F)c(F)c2)C(=O)N1] |
| | 3-[4-[3,3-difluoro-1-[2-[1-[(1R,3S)-3-[[5-fluoro-4-[3-(2-oxo-1-pyridyl)phenyl]pyrimidin-2-yl]amino]cyclohexane- |
| | carbonyl]-4-piperidyl]ethyl]-4-piperidyl]-3-fluoro-anilino]piperidine-2,6-dione |
| 311 | 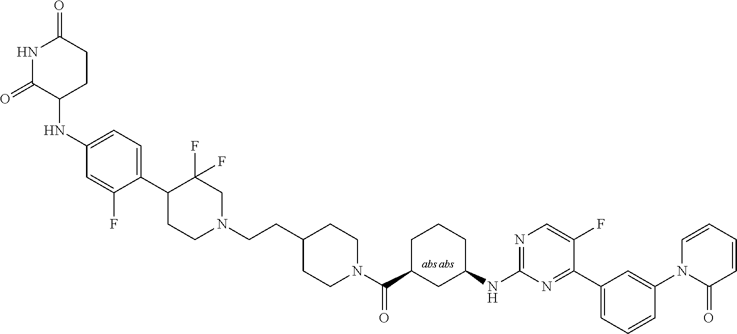 [SMILES: O=C1CCC(Nc2ccc(C3CCN(CCC4CCN(C(=O)[C@H]5CCC[C@@H](Nc6ncc(F)c(-c7cccc(-n8ccccc8=O)c7)n6)C5)CC4)CC3(F)F)c(F)c2)C(=O)N1] |
| | 3-((4-(3,3-difluoro-1-(2-(1-((1S,3R)-3-((5-fluoro-4-(3-(2-oxopyridin-1(2H)-yl)phenyl)pyrimidin-2-yl)amino)cyclohexane- |
| | 1-carbonyl)piperidin-4-yl)ethyl)piperidin-4-yl)-3-fluorophenyl)amino)piperidine-2,6-dione |
| 312 | 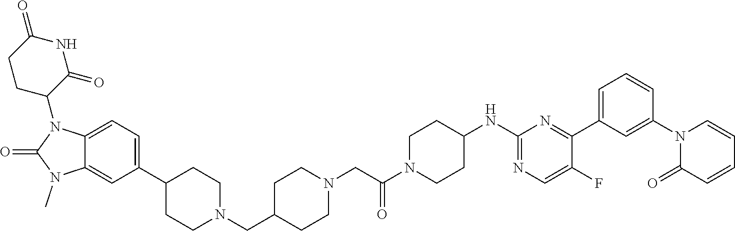 [SMILES: Cn1c(=O)n(C2CCC(=O)NC2=O)c2ccc(C3CCN(CC4CCN(CC(=O)N5CCC(Nc6ncc(F)c(-c7cccc(-n8ccccc8=O)c7)n6)CC5)CC4)CC3)cc21] |
| | 3-(5-(1-((1-(2-(4-((5-fluoro-4-(3-(2-oxopyridin-1(2H)-yl)phenyl)pyrimidin-2-yl)amino)piperidin-1-yl)-2-oxoethyl)- |
| | piperidin-4-yl)methyl)piperidin-4-yl)-3-methyl-2-oxo-2,3-dihydro-1H-benzo[d]imidazol-1-yl)piperidine-2,6-dione |
| 313 | 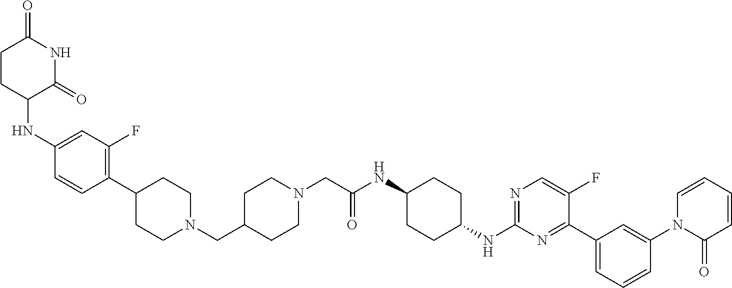 [SMILES: O=C1CCC(Nc2ccc(C3CCN(CC4CCN(CC(=O)N[C@H]5CC[C@H](Nc6ncc(F)c(-c7cccc(-n8ccccc8=O)c7)n6)CC5)CC4)CC3)c(F)c2)C(=O)N1] |
| | trans-2-(4-((4-(4-((2,6-dioxopiperidin-3-yl)amino)-2-fluorophenyl)piperidin-1-yl)methyl)piperidin-1-yl)-N-(4-((5-fluoro- |
| | 4-(3-(2-oxopyridin-1(2H)-yl)phenyl)pyrimidin-2-yl)amino)cyclohexyl)acetamide |
| 314 | 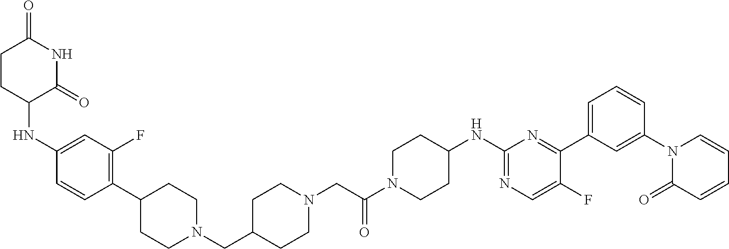 [SMILES: O=C1CCC(Nc2ccc(C3CCN(CC4CCN(CC(=O)N5CCC(Nc6ncc(F)c(-c7cccc(-n8ccccc8=O)c7)n6)CC5)CC4)CC3)c(F)c2)C(=O)N1] |
| | 3-((3-fluoro-4-(1-((1-(2-(4-((5-fluoro-4-(3-(2-oxopyridin-1(2H)-yl)phenyl)pyrimidin-2-yl)amino)piperidin-1-yl)- |
| | 2-oxoethyl)piperidin-4-yl)methyl)piperidin-4-yl)phenyl)amino)piperidine-2,6-dione |
| 315 | 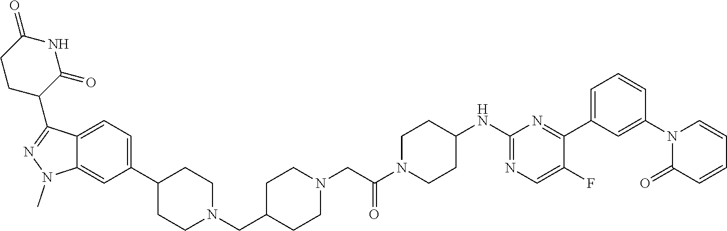 [SMILES: Cn1nc(C2CCC(=O)NC2=O)c2ccc(C3CCN(CC4CCN(CC(=O)N5CCC(Nc6ncc(F)c(-c7cccc(-n8ccccc8=O)c7)n6)CC5)CC4)CC3)cc21] |
| | 3-(6-(1-((1-(2-(4-((5-fluoro-4-(3-(2-oxopyridin-1(2H)-yl)phenyl)pyrimidin-2-yl)amino)piperidin-1-yl)-2-oxoethyl) |
| | piperidin-4-yl)methyl)piperidin-4-yl)-1-methyl-1H-indazol-3-yl)piperidine-2,6-dione |
| 316 | 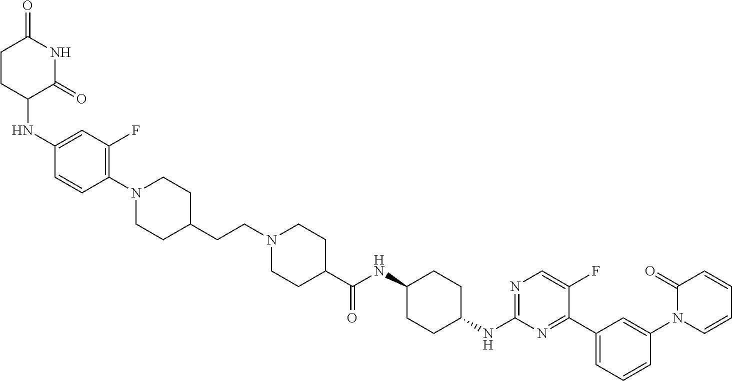 [SMILES: O=C1CCC(Nc2ccc(N3CCC(CCN4CCC(C(=O)N[C@H]5CC[C@H](Nc6ncc(F)c(-c7cccc(-n8ccccc8=O)c7)n6)CC5)CC4)CC3)c(F)c2)C(=O)N1] |
| | trans-1-[2-[1-[4-[(2,6-dioxo-3-piperidyl)amino]-2-fluoro-phenyl]-4-piperidyl]ethyl]-N-[4-[[5-fluoro-4-[3-(2-oxo-1- |
| | pyridyl)phenyl]pyrimidin-2-yl]amino]cyclohexyl]piperidine-4-carboxamide |
| 317 | 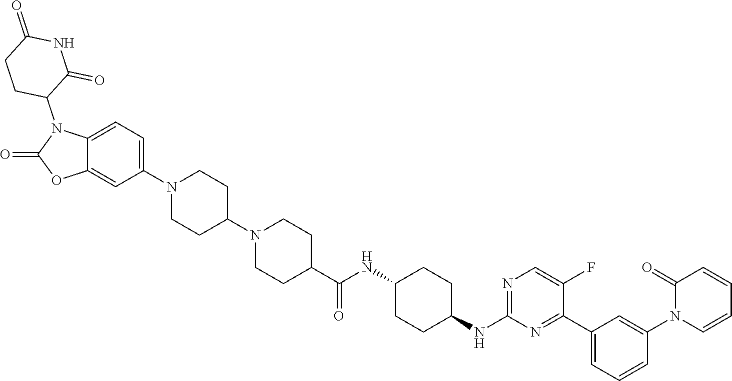 [SMILES: O=C1CCC(n2c(=O)oc3cc(N4CCC(N5CCC(C(=O)N[C@H]6CC[C@H](Nc7ncc(F)c(-c8cccc(-n9ccccc9=O)c8)n7)CC6)CC5)CC4)ccc32)C(=O)N1] |
| | trans-1′-(3-(2,6-dioxopiperidin-3-yl)-2-oxo-2,3-dihydrobenzo[d]oxazol-6-yl)-N-(4-((5-fluoro-4-(3-(2-oxopyridin-1(2H)- |
| | yl)phenyl)pyrimidin-2-yl)amino)cyclohexyl)-[1,4′-bipiperidine]-4-carboxamide |
| 318 | 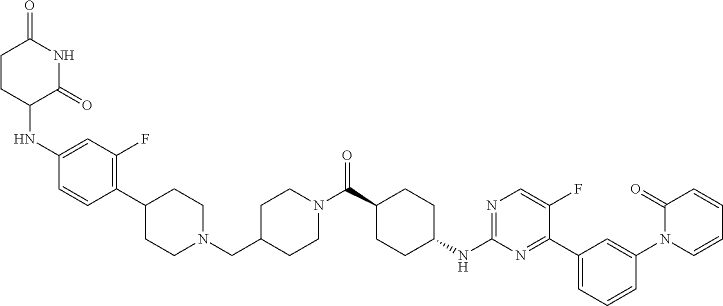 [SMILES: O=C1CCC(Nc2ccc(C3CCN(CC4CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-3-((3-fluoro-4-(1-((1-(4-((5-fluoro-4-(3-(2-oxopyridin-1(2H)-yl)phenyl)pyrimidin-2-yl)amino)cyclohexane- |
| | 1-carbonyl)piperidin-4-yl)methyl)piperidin-4-yl)phenyl)amino)piperidine-2,6-dione |
| 319 | 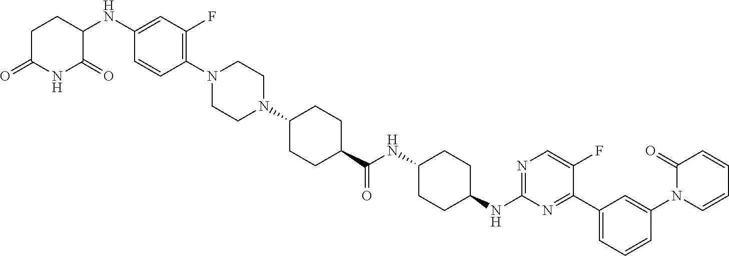 [SMILES: O=C1CCC(Nc2ccc(N3CCN([C@H]4CC[C@H](C(=O)N[C@H]5CC[C@H](Nc6ncc(F)c(-c7cccc(-n8ccccc8=O)c7)n6)CC5)CC4)CC3)c(F)c2)C(=O)N1] |
| | trans-trans-4-(4-(4-((2,6-dioxopiperidin-3-yl)amino)-2-fluorophenyl)piperazin-1-yl)-N-(4-((5-fluoro-4-(3-(2-oxopyridin- |
| | 1(2H)-yl)phenyl)pyrimidin-2-yl)amino)cyclohexyl)cyclohexane-1-carboxamide |
| 320 | 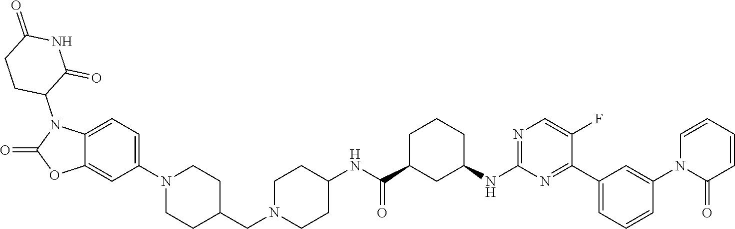 [SMILES: O=C1CCC(n2c(=O)oc3cc(N4CCC(CN5CCC(NC(=O)[C@H]6CCC[C@@H](Nc7ncc(F)c(-c8cccc(-n9ccccc9=O)c8)n7)C6)CC5)CC4)ccc32)C(=O)N1] |
| | cis-N-(1-((1-(3-(2,6-dioxopiperidin-3-yl)-2-oxo-2,3-dihydrobenzo[d]oxazol-6-yl)piperidin-4-yl)methyl)piperidin-4-yl)- |
| | 3-((5-fluoro-4-(3-(2-oxopyridin-1(2H)-yl)phenyl)pyrimidin-2-yl)amino)cyclohexane-1-carboxamide |
| 321 | 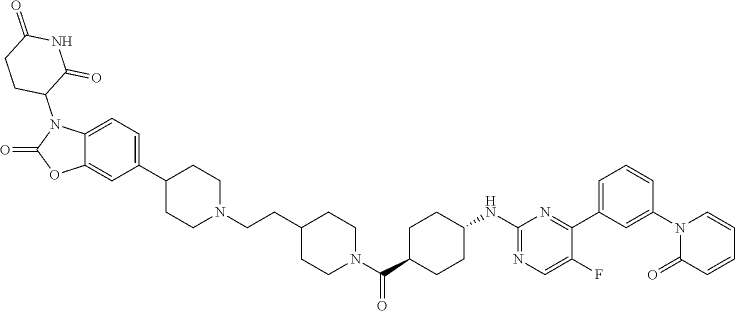 [SMILES: O=C1CCC(n2c(=O)oc3cc(C4CCN(CCC5CCN(C(=O)[C@H]6CC[C@H](Nc7ncc(F)c(-c8cccc(-n9ccccc9=O)c8)n7)CC6)CC5)CC4)ccc32)C(=O)N1] |
| | trans-3-(6-(1-(2-(1-(4-((5-fluoro-4-(3-(2-oxopyridin-1(2H)-yl)phenyl)pyrimidin-2-yl)amino)cyclohexane-1-carbonyl)- |
| | piperidin-4-yl)ethyl)piperidin-4-yl)-2-oxobenzo[d]oxazol-3(2H)-yl)piperidine-2,6-dione |
| 322 | 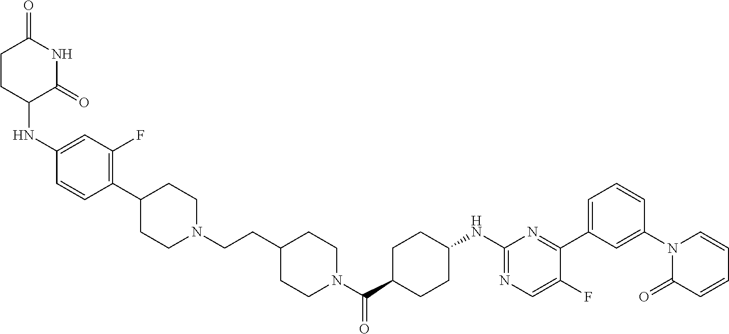 [SMILES: O=C1CCC(Nc2ccc(C3CCN(CCC4CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-3-((3-fluoro-4-(1-(2-(1-((1r,4r)-4-((5-fluoro-4-(3-(2-oxopyridin-1(2H)-yl)phenyl)pyrimidin-2-yl)amino)cyclo- |
| | hexane-1-carbonyl)piperidin-4-yl)ethyl)piperidin-4-yl)phenyl)amino)piperidine-2,6-dione |
| 323 | 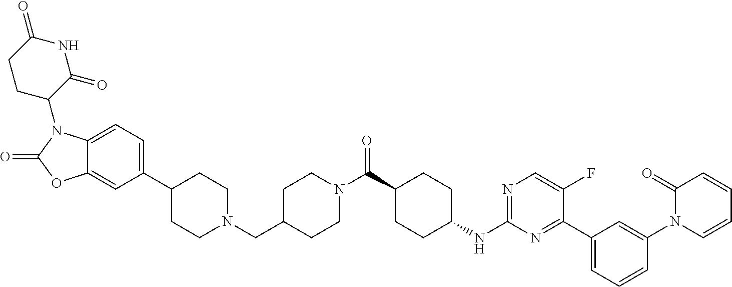 [SMILES: O=C1CCC(n2c(=O)oc3cc(C4CCN(CC5CCN(C(=O)[C@H]6CC[C@H](Nc7ncc(F)c(-c8cccc(-n9ccccc9=O)c8)n7)CC6)CC5)CC4)ccc32)C(=O)N1] |
| | trans-3-(6-(1-((1-(4-((5-fluoro-4-(3-(2-oxopyridin-1(2H)-yl)phenyl)pyrimidin-2-yl)amino)cyclohexane-1-carbonyl)- |
| | piperidin-4-yl)methyl)piperidin-4-yl)-2-oxobenzo[d]oxazol-3(2H)-yl)piperidine-2,6-dione |
| 324 | 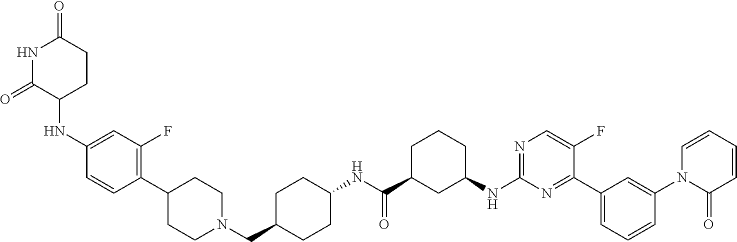 [SMILES: O=C1CCC(Nc2ccc(C3CCN(C[C@H]4CC[C@H](NC(=O)[C@H]5CCC[C@@H](Nc6ncc(F)c(-c7cccc(-n8ccccc8=O)c7)n6)C5)CC4)CC3)c(F)c2)C(=O)N1] |
| | trans-N-(cis-4-((4-(4-((2,6-dioxopiperidin-3-yl)amino)-2-fluorophenyl)piperidin-1-yl)methyl)cyclohexyl)-3-((5-fluoro- |
| | 4-(3-(2-oxopyridin-1(2H)-yl)phenyl)pyrimidin-2-yl)amino)cyclohexane-1-carboxamide |
| 325 | 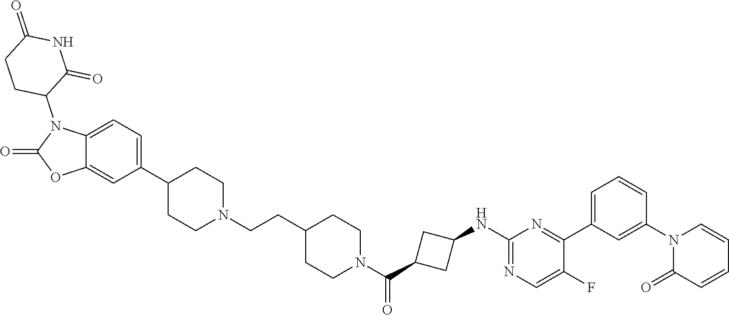 [SMILES: O=C1CCC(n2c(=O)oc3cc(C4CCN(CCC5CCN(C(=O)[C@H]6C[C@@H](Nc7ncc(F)c(-c8cccc(-n9ccccc9=O)c8)n7)C6)CC5)CC4)ccc32)C(=O)N1] |
| | cis-3-(6-(1-(2-(1-(3-((5-fluoro-4-(3-(2-oxopyridin-1(2H)-yl)phenyl)pyrimidin-2-yl)amino)cyclobutane-1-carbonyl) |
| | piperidin-4-yl)ethyl)piperidin-4-yl)-2-oxobenzo[d]oxazol-3(2H)-yl)piperidine-2,6-dione |
| 326 | 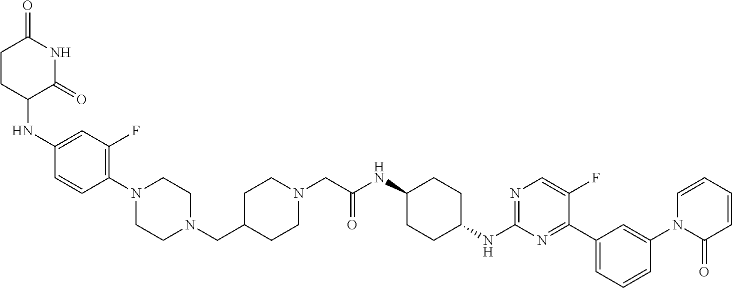 [SMILES: O=C1CCC(Nc2ccc(N3CCN(CC4CCN(CC(=O)N[C@H]5CC[C@H](Nc6ncc(F)c(-c7cccc(-n8ccccc8=O)c7)n6)CC5)CC4)CC3)c(F)c2)C(=O)N1] |
| | trans-2-(4-((4-(4-((2,6-dioxopiperidin-3-yl)amino)-2-fluorophenyl)piperazin-1-yl)methyl)piperidin-1-yl)-N-(4-((5- |
| | fluoro-4-(3-(2-oxopyridin-1(2H)-yl)phenyl)pyrimidin-2-yl)amino)cyclohexyl)acetamide |
| 327 | 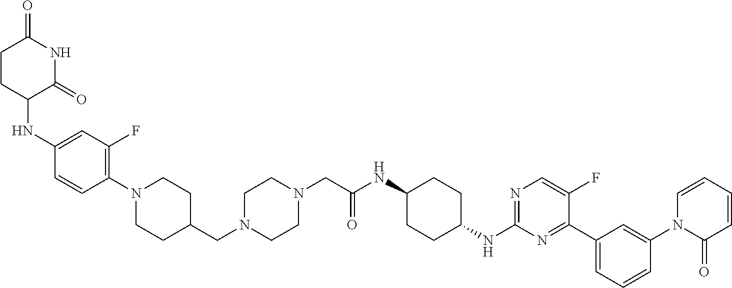 [SMILES: O=C1CCC(Nc2ccc(N3CCC(CN4CCN(CC(=O)N[C@H]5CC[C@H](Nc6ncc(F)c(-c7cccc(-n8ccccc8=O)c7)n6)CC5)CC4)CC3)c(F)c2)C(=O)N1] |
| | trans-2-[4-[[1-[4-[(2,6-dioxo-3-piperidyl)amino]-2-fluoro-phenyl]-4-piperidyl]methyl]piperazin-1-yl]-N-[4-[[5-fluoro- |
| | 4-[3-(2-oxo-1-pyridyl)phenyl]pyrimidin-2-yl]amino]cyclohexyl]acetamide |
| 328 | 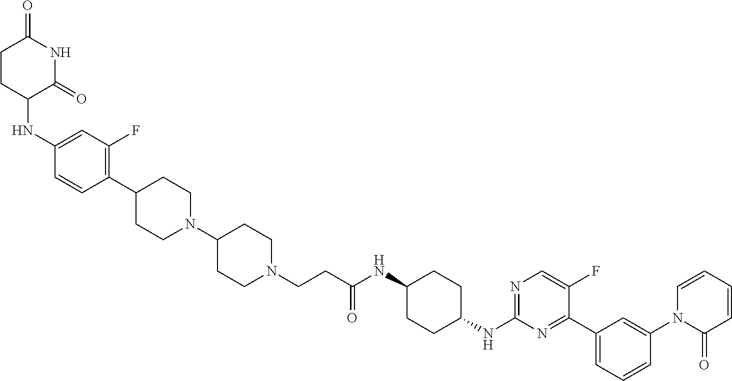 [SMILES: O=C1CCC(Nc2ccc(C3CCN(C4CCN(CCC(=O)N[C@H]5CC[C@H](Nc6ncc(F)c(-c7cccc(-n8ccccc8=O)c7)n6)CC5)CC4)CC3)c(F)c2)C(=O)N1] |
| | trans-3-(4-(4-((2,6-dioxopiperidin-3-yl)amino)-2-fluorophenyl)-[1,4′-bipiperidin]-1′-yl)-N-(4-((5-fluoro-4-(3-(2- |
| | oxopyridin-1(2H)-yl)phenyl)pyrimidin-2-yl)amino)cyclohexyl)propanamide |
| 329 | 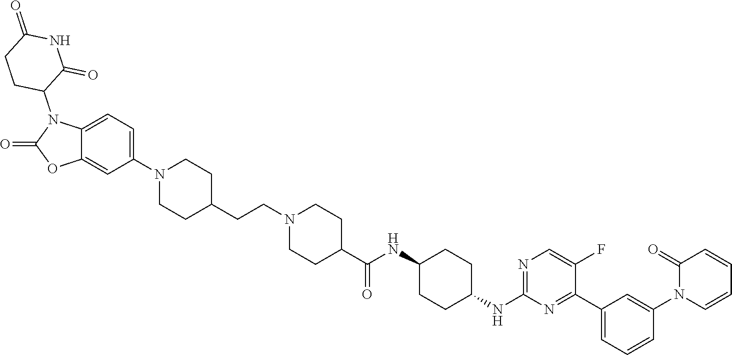 [SMILES: O=C1CCC(n2c(=O)oc3cc(N4CCC(CCN5CCC(C(=O)N[C@H]6CC[C@H](Nc7ncc(F)c(-c8cccc(-n9ccccc9=O)c8)n7)CC6)CC5)CC4)ccc32)C(=O)N1] |
| | trans-1-[2-[1-[3-(2,6-dioxo-3-piperidyl)-2-oxo-1,3-benzoxazol-6-yl]-4-piperidyl]ethyl]-N-[4-[[5-fluoro-4-[3-(2-oxo- |
| | 1-pyridyl)phenyl]pyrimidin-2-yl]amino]cyclohexyl]piperidine-4-carboxamide |
| 330 | 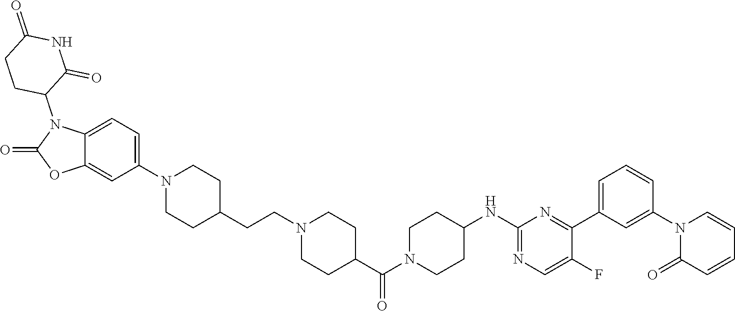 [SMILES: O=C1CCC(n2c(=O)oc3cc(N4CCC(CCN5CCC(C(=O)N6CCC(Nc7ncc(F)c(-c8cccc(-n9ccccc9=O)c8)n7)CC6)CC5)CC4)ccc32)C(=O)N1] |
| | 3-[6-[4-[2-[4-[4-[5-fluoro-4-[3-(2-oxo-1-pyridyl)phenyl]pyrimidin-2-yl]amino]piperidine-1-carbonyl]-1-piperidyl] |
| | ethyl]-1-piperidyl]-2-oxo-1,3-benzoxazol-3-yl]piperidine-2,6-dione |
| 331 | 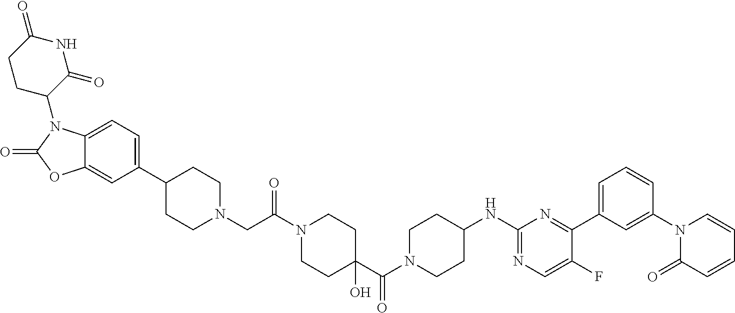 [SMILES: O=C1CCC(n2c(=O)oc3cc(C4CCN(CC(=O)N5CCC(O)(C(=O)N6CCC(Nc7ncc(F)c(-c8cccc(-n9ccccc9=O)c8)n7)CC6)CC5)CC4)ccc32)C(=O)N1] |
| | 3-(6-(1-(2-(4-(4-((5-fluoro-4-(3-(2-oxopyridin-1(2H)-yl)phenyl)pyrimidin-2-yl)amino)piperidine-1-carbonyl)-4-hydroxy- |
| | piperidin-1-yl)-2-oxoethyl)piperidin-4-yl)-2-oxobenzo[d]oxazol-3(2H)-yl)piperidine-2,6-dione |
| 332 | 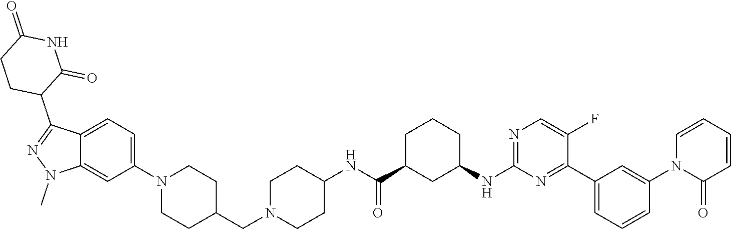 [SMILES: Cn1nc(C2CCC(=O)NC2=O)c2ccc(N3CCC(CN4CCC(NC(=O)[C@H]5CCC[C@@H](Nc6ncc(F)c(-c7cccc(-n8ccccc8=O)c7)n6)C5)CC4)CC3)cc21] |
| | cis-N-(1-((1-(3-(2,6-dioxopiperidin-3-yl)-1-methyl-1H-indazol-6-yl)piperidin-4-yl)methyl)piperidin-4-yl)-3-((5-fluoro- |
| | 4-(3-(2-oxopyridin-1(2H)-yl)phenyl)pyrimidin-2-yl)amino)cyclohexane-1-carboxamide |
| 333 | 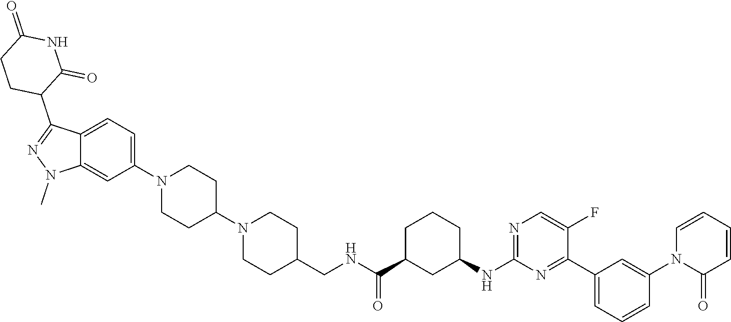 [SMILES: Cn1nc(C2CCC(=O)NC2=O)c2ccc(N3CCC(N4CCC(CNC(=O)[C@H]5CCC[C@@H](Nc6ncc(F)c(-c7cccc(-n8ccccc8=O)c7)n6)C5)CC4)CC3)cc21] |
| | cis-N-((1′-(3-(2,6-dioxopiperidin-3-yl)-1-methyl-1H-indazol-6-yl)-[1,4′-bipiperidin]-4-yl)methyl)-3-((5-fluoro-4-(3- |
| | (2-oxopyridin-1(2H)-yl)phenyl)pyrimidin-2-yl)amino)cyclohexane-1-carboxamide |
| 334 | 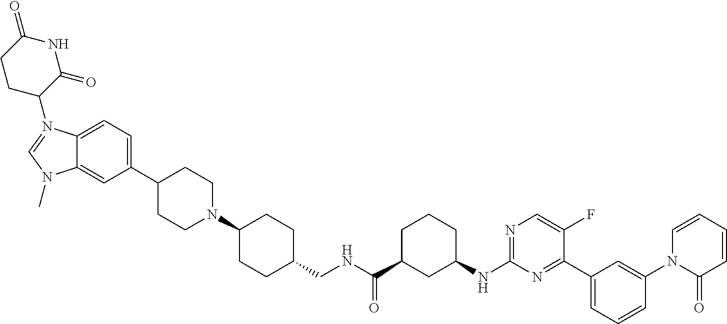 |
| | cis-trans-N-((4-(4-(3-(2,6-dioxopiperidin-3-yl)-1-methyl-1H-indazol-6-yl)piperidin-1-yl)cyclohexyl)methyl)-3-((5- |
| | fluoro-4-(3-(2-oxopyridin-1(2H)-yl)phenyl)pyrimidin-2-yl)amino)cyclohexane-1-carboxamide |
| 335 | 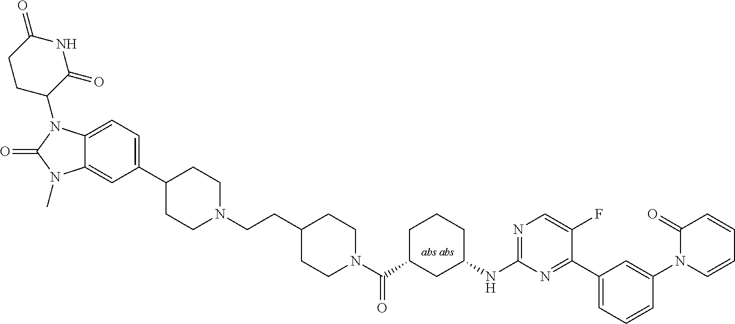 [SMILES: Cn1c(=O)n(C2CCC(=O)NC2=O)c2ccc(C3CCN(CCC4CCN(C(=O)[C@@H]5CCC[C@H](Nc6ncc(F)c(-c7cccc(-n8ccccc8=O)c7)n6)C5)CC4)CC3)cc21] |
| | 3-(5-(1-(2-(1-((1R,3S)-3-((5-fluoro-4-(3-(2-oxopyridin-1(2H)-yl)phenyl)pyrimidin-2-yl)amino)cyclohexane-1-carbon- |
| | yl)piperidin-4-yl)ethyl)piperidin-4-yl)-3-methyl-2-oxo-2,3-dihydro-1H-benzo[d]imidazol-1-yl)piperidine-2,6-dione |
| 336 | 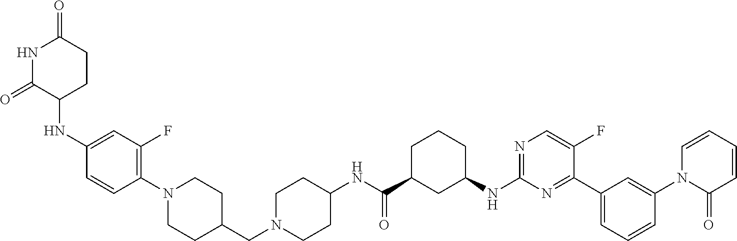 [SMILES: O=C1CCC(Nc2ccc(N3CCC(CN4CCC(NC(=O)[C@H]5CCC[C@@H](Nc6ncc(F)c(-c7cccc(-n8ccccc8=O)c7)n6)C5)CC4)CC3)c(F)c2)C(=O)N1] |
| | cis-N-(1-((1-(4-((2,6-dioxopiperidin-3-yl)amino)-2-fluorophenyl)piperidin-4-yl)methyl)piperidin-4-yl)-3-((5-fluoro- |
| | 4-(3-(2-oxopyridin-1(2H)-yl)phenyl)pyrimidin-2-yl)amino)cyclohexane-1-carboxamide |
| 337 | 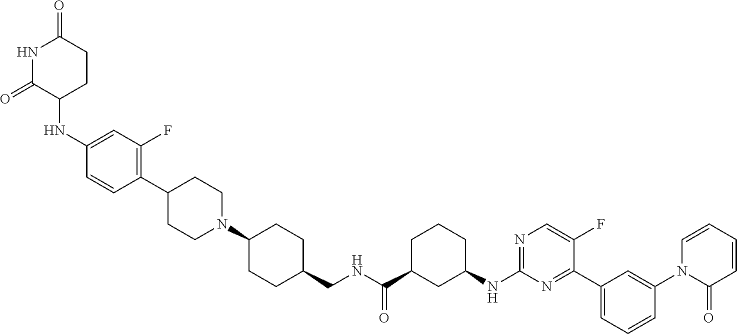 [SMILES: O=C1CCC(Nc2ccc(C3CCN([C@H]4CC[C@@H](CNC(=O)[C@H]5CCC[C@@H](Nc6ncc(F)c(-c7cccc(-n8ccccc8=O)c7)n6)C5)CC4)CC3)c(F)c2)C(=O)N1] |
| | cis-N-((cis-4-(4-(4-((2,6-dioxopiperidin-3-yl)amino)-2-fluorophenyl)piperidin-1-yl)cyclohexyl)methyl)-3-((5-fluoro-4- |
| | (3-(2-oxopyridin-1(2H)-yl)phenyl)pyrimidin-2-yl)amino)cyclohexane-1-carboxamide |
| 338 | 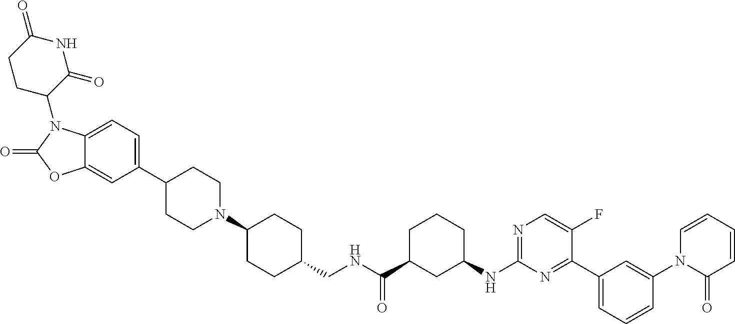 [SMILES: O=C1CCC(n2c(=O)oc3cc(C4CCN([C@H]5CC[C@H](CNC(=O)[C@H]6CCC[C@@H](Nc7ncc(F)c(-c8cccc(-n9ccccc9=O)c8)n7)C6)CC5)CC4)ccc32)C(=O)N1] |
| | trans-3-(5-(1-(2-(4-(4-((5-chloro-4-(4′-fluoro-[1,1′-biphenyl]-3-yl)pyrimidin-2-yl)amino)piperidine-1-carbonyl)cyclo- |
| | hexyl)ethyl)piperidin-4-yl)-3-methyl-2-oxo-2,3-dihydro-1H-benzo[d]imidazol-1-yl)piperidine-2,6-dione |
| 339 | 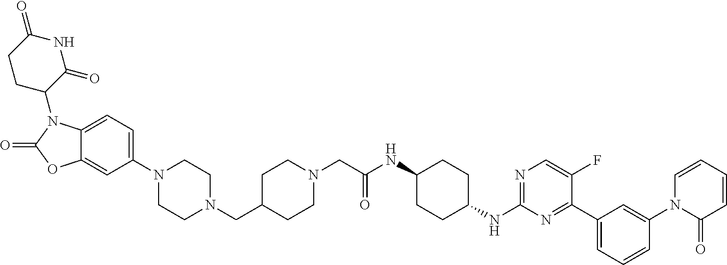 [SMILES: O=C1CCC(n2c(=O)oc3cc(N4CCN(CC5CCN(CC(=O)N[C@H]6CC[C@H](Nc7ncc(F)c(-c8cccc(-n9ccccc9=O)c8)n7)CC6)CC5)CC4)ccc32)C(=O)N1] |
| | trans-2-(4-((4-(3-(2,6-dioxopiperidin-3-yl)-2-oxo-2,3-dihydrobenzo[d]oxazol-6-yl)piperazin-1-yl)methyl)piperidin-1- |
| | yl)-N-(4-((5-fluoro-4-(3-(2-oxopyridin-1(2H)-yl)phenyl)pyrimidin-2-yl)amino)cyclohexyl)acetamide |
| 340 | 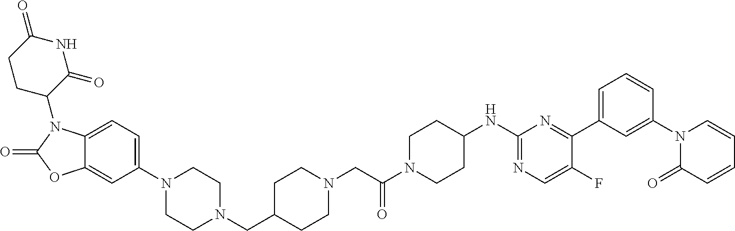 [SMILES: O=C1CCC(n2c(=O)oc3cc(N4CCN(CC5CCN(CC(=O)N6CCC(Nc7ncc(F)c(-c8cccc(-n9ccccc9=O)c8)n7)CC6)CC5)CC4)ccc32)C(=O)N1] |
| | 3-(6-(4-((1-(2-(4-((5-fluoro-4-(3-(2-oxopyridin-1(2H)-yl)phenyl)pyrimidin-2-yl)amino)piperidin-1-yl)-2-oxoethyl)- |
| | piperidin-4-yl)methyl)piperazin-1-yl)-2-oxobenzo[d]oxazol-3(2H)-yl)piperidine-2,6-dione |
| 341 | 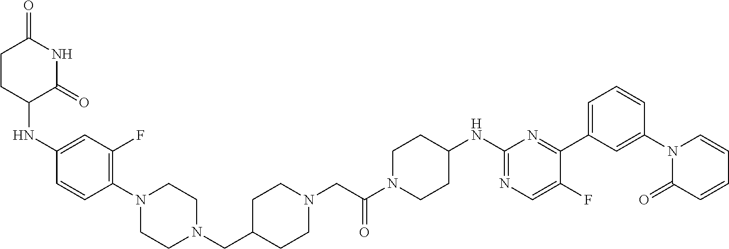 [SMILES: O=C1CCC(Nc2ccc(N3CCN(CC4CCN(CC(=O)N5CCC(Nc6ncc(F)c(-c7cccc(-n8ccccc8=O)c7)n6)CC5)CC4)CC3)c(F)c2)C(=O)N1] |
| | 3-((3-fluoro-4-(4-((1-(2-(4-((5-fluoro-4-(3-(2-oxopyridin-1(2H)-yl)phenyl)pyrimidin-2-yl)amino)piperidin-1-yl)-2- |
| | oxoethyl)piperidin-4-yl)methyl)piperazin-1-yl)phenyl)amino)piperidine-2,6-dione |
| 342 | 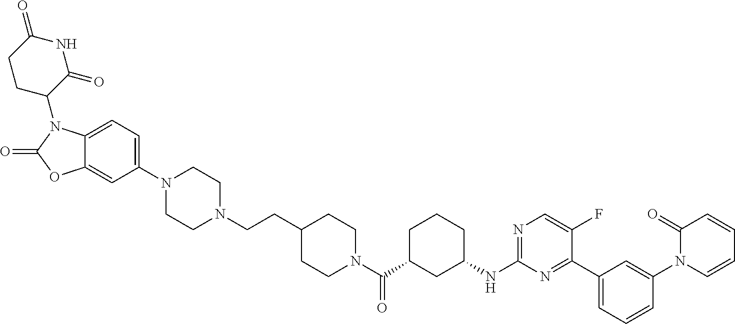 [SMILES: O=C1CCC(n2c(=O)oc3cc(N4CCN(CCC5CCN(C(=O)[C@@H]6CCC[C@H](Nc7ncc(F)c(-c8cccc(-n9ccccc9=O)c8)n7)C6)CC5)CC4)ccc32)C(=O)N1] |
| | cis-3-(6-(4-(2-(1-(3-((5-fluoro-4-(3-(2-oxopyridin-1(2H)-yl)phenyl)pyrimidin-2-yl)amino)cyclohexane-1-carbonyl)- |
| | piperidin-4-yl)ethyl)piperazin-1-yl)-2-oxobenzo[d]oxazol-3(2H)-yl)piperidine-2,6-dione |
| 343 | 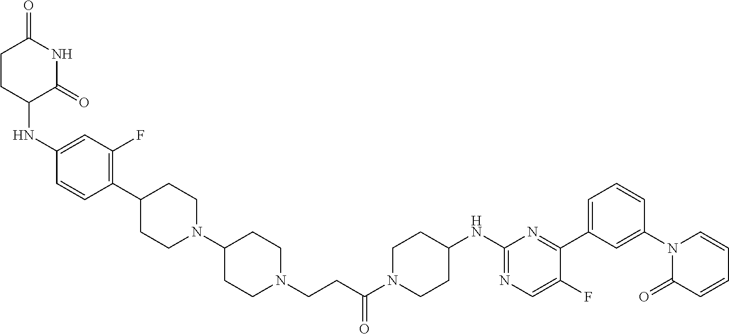 [SMILES: O=C1CCC(Nc2ccc(C3CCN(C4CCN(CCC(=O)N5CCC(Nc6ncc(F)c(-c7cccc(-n8ccccc8=O)c7)n6)CC5)CC4)CC3)c(F)c2)C(=O)N1] |
| | 3-((3-fluoro-4-(1′-(3-(4-((5-fluoro-4-(3-(2-oxopyridin-1(2H)-yl)phenyl)pyrimidin-2-yl)amino)piperidin-1-yl)-3- |
| | oxopropyl)-[1,4′-bipiperidin]-4-yl)phenyl)amino)piperidine-2,6-dione |
| 344 | 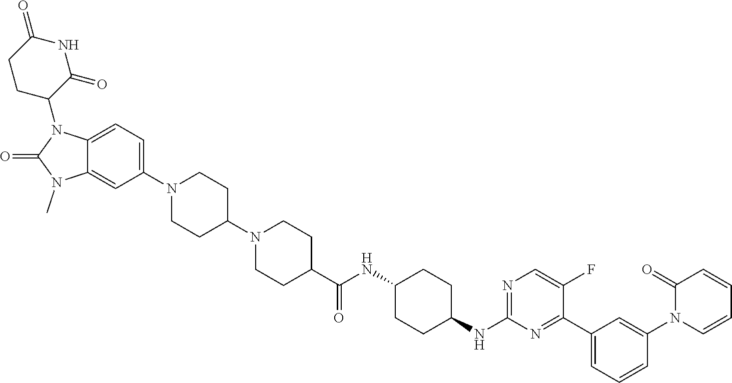 [SMILES: Cn1c(=O)n(C2CCC(=O)NC2=O)c2ccc(N3CCC(N4CCC(C(=O)N[C@H]5CC[C@H](Nc6ncc(F)c(-c7cccc(-n8ccccc8=O)c7)n6)CC5)CC4)CC3)cc21] |
| | trans-1′-(1-(2,6-dioxopiperidin-3-yl)-3-methyl-2-oxo-2,3-dihydro-1H-benzo[d]imidazol-5-yl)-N-(4-((5-fluoro-4-(3- |
| | (2-oxopyridin-1(2H)-yl)phenyl)pyrimidin-2-yl)amino)cyclohexyl)-[1,4′-bipiperidine]-4-carboxamide |
| 345 | 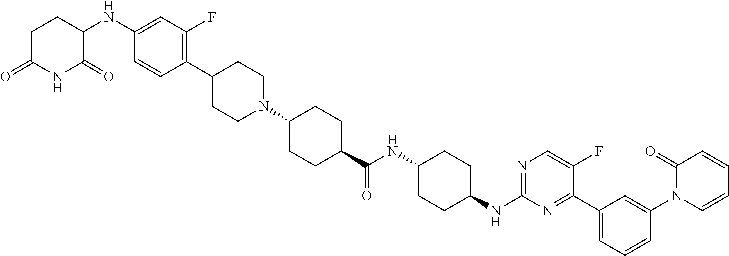 [SMILES: O=C1CCC(Nc2ccc(C3CCN([C@H]4CC[C@H](C(=O)N[C@H]5CC[C@H](Nc6ncc(F)c(-c7cccc(-n8ccccc8=O)c7)n6)CC5)CC4)CC3)c(F)c2)C(=O)N1] |
| | trans-4-(4-(4-((2,6-dioxopiperidin-3-yl)amino)-2-fluorophenyl)piperidin-1-yl)-N-(trans-4-((5-fluoro-4-(3-(2-oxo- |
| | pyridin-1(2H)-yl)phenyl)pyrimidin-2-yl)amino)cyclohexyl)cyclohexane-1-carboxamide |
| 346 | 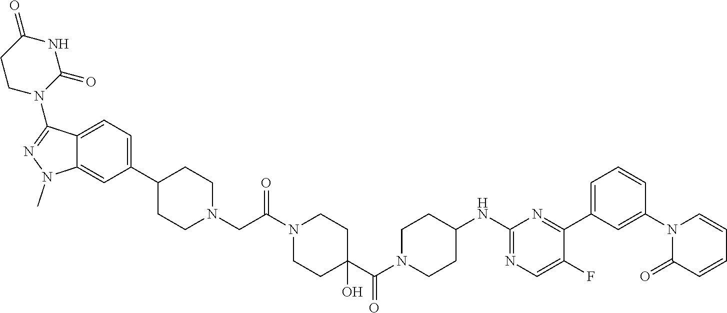 [SMILES: Cn1nc(N2CCC(=O)NC2=O)c2ccc(C3CCN(CC(=O)N4CCC(O)(C(=O)N5CCC(Nc6ncc(F)c(-c7cccc(-n8ccccc8=O)c7)n6)CC5)CC4)CC3)cc21] |
| | 1-(6-(1-(2-(4-(4-((5-fluoro-4-(3-(2-oxopyridin-1(2H)-yl)phenyl)pyrimidin-2-yl)amino)piperidine-1-carbonyl)-4- |
| | hydroxypiperidin-1-yl)-2-oxoethyl)piperidin-4-yl)-1-methyl-1H-indazol-3-yl)dihydropyrimidine-2,4(1H,3H)-dione |
| 347 | 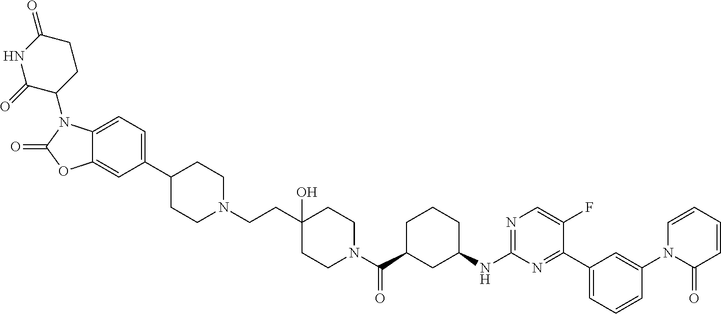 [SMILES: O=C1CCC(n2c(=O)oc3cc(C4CCN(CCC5(O)CCN(C(=O)[C@H]6CCC[C@@H](Nc7ncc(F)c(-c8cccc(-n9ccccc9=O)c8)n7)C6)CC5)CC4)ccc32)C(=O)N1] |
| | cis-3-(6-(1-(2-(1-(3-((5-fluoro-4-(3-(2-oxopyridin-1(2H)-yl)phenyl)pyrimidin-2-yl)amino)cyclohexane-1-carbonyl)- |
| | 4-hydroxypiperidin-4-yl)ethyl)piperidin-4-yl)-2-oxobenzo[d]oxazol-3(2H)-yl)piperidine-2,6-dione |
| 348 | 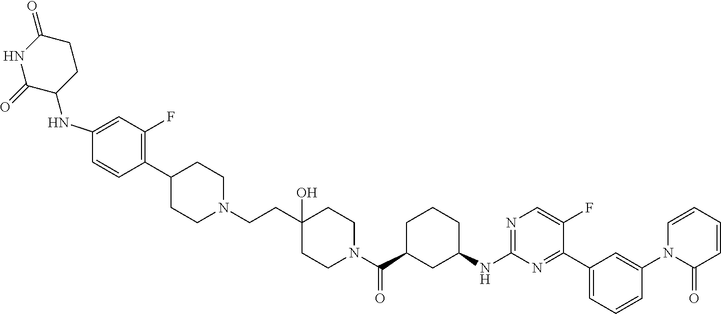 [SMILES: O=C1CCC(Nc2ccc(C3CCN(CCC4(O)CCN(C(=O)[C@H]5CCC[C@@H](Nc6ncc(F)c(-c7cccc(-n8ccccc8=O)c7)n6)C5)CC4)CC3)c(F)c2)C(=O)N1] |
| | cis-3-((3-fluoro-4-(1-(2-(1-(3-((5-fluoro-4-(3-(2-oxopyridin-1(2H)-yl)phenyl)pyrimidin-2-yl)amino)cyclohexane- |
| | 1-carbonyl)-4-hydroxypiperidin-4-yl)ethyl)piperidin-4-yl)phenyl)amino)piperidine-2,6-dione |
| 349 | 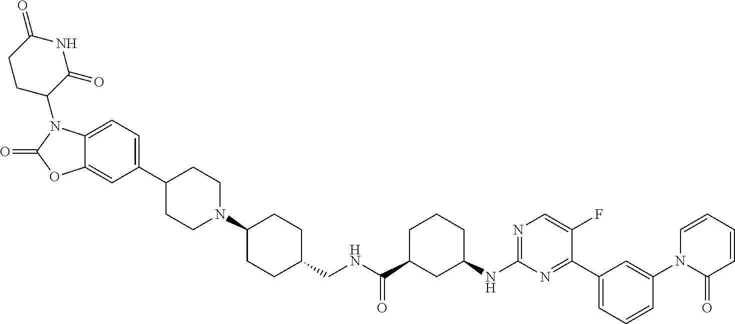 [SMILES: O=C1CCC(n2c(=O)oc3cc(C4CCN([C@H]5CC[C@H](CNC(=O)[C@H]6CCC[C@@H](Nc7ncc(F)c(-c8cccc(-n9ccccc9=O)c8)n7)C6)CC5)CC4)ccc32)C(=O)N1] |
| | cis-trans-N-((4-(4-(3-(2,6-dioxopiperidin-3-yl)-2-oxo-2,3-dihydrobenzo[d]oxazol-6-yl)piperidin-1-yl)cyclohexyl)meth- |
| | yl)-3-((5-fluoro-4-(3-(2-oxopyridin-1(2H)-yl)phenyl)pyrimidin-2-yl)amino)cyclohexane-1-carboxamide |
| 350 | 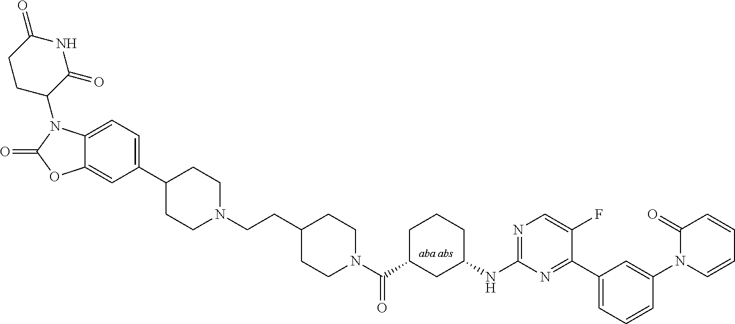 [SMILES: O=C1CCC(n2c(=O)oc3cc(C4CCN(CCC5CCN(C(=O)[C@@H]6CCC[C@H](Nc7ncc(F)c(-c8cccc(-n9ccccc9=O)c8)n7)C6)CC5)CC4)ccc32)C(=O)N1] |
| | 3-(6-(1-(2-(1-((1R,3S)-3-((5-fluoro-4-(3-(2-oxopyridin-1(2H)-yl)phenyl)pyrimidin-2-yl)amino)cyclohexane-1-carbon- |
| | yl)piperidin-4-yl)ethyl)piperidin-4-yl)-2-oxobenzo[d]oxazol-3(2H)-yl)piperidine-2,6-dione |
| 351 | 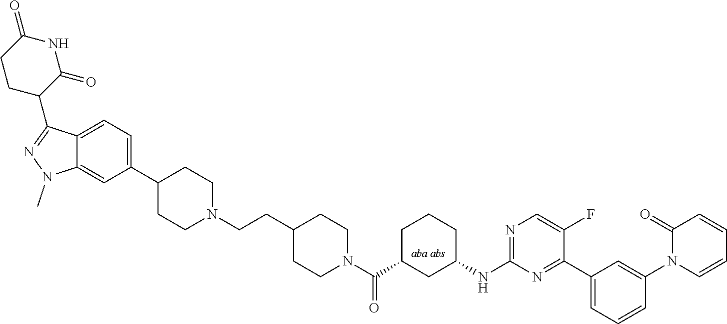 [SMILES: Cn1nc(C2CCC(=O)NC2=O)c2ccc(C3CCN(CCC4CCN(C(=O)[C@@H]5CCC[C@H](Nc6ncc(F)c(-c7cccc(-n8ccccc8=O)c7)n6)C5)CC4)CC3)cc21] |
| | 3-(6-(1-(2-(1-((1R,3S)-3-((5-fluoro-4-(3-(2-oxopyridin-1(2H)-yl)phenyl)pyrimidin-2-yl)amino)cyclohexane-1- |
| | carbonyl)piperidin-4-yl)ethyl)piperidin-4-yl)-1-methyl-1H-indazol-3-yl)piperidine-2,6-dione |
| 352 | 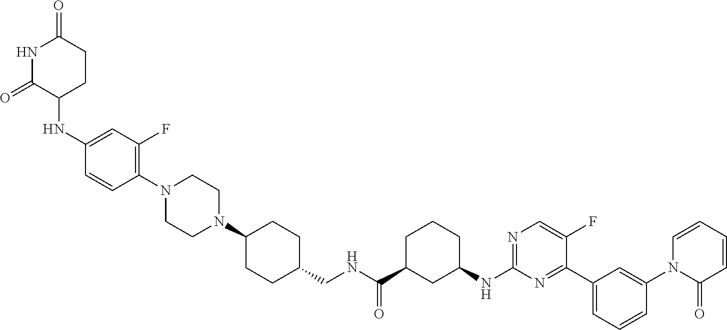 [SMILES: O=C1CCC(Nc2ccc(N3CCN([C@H]4CC[C@H](CNC(=O)[C@H]5CCC[C@@H](Nc6ncc(F)c(-c7cccc(-n8ccccc8=O)c7)n6)C5)CC4)CC3)c(F)c2)C(=O)N1] |
| | cis-N-((trans-4-(4-(4-((2,6-dioxopiperidin-3-yl)amino)-2-fluorophenyl)piperazin-1-yl)cyclohexyl)methyl)-3-((5- |
| | fluoro-4-(3-(2-oxopyridin-1(2H)-yl)phenyl)pyrimidin-2-yl)amino)cyclohexane-1-carboxamide |
| 353 | 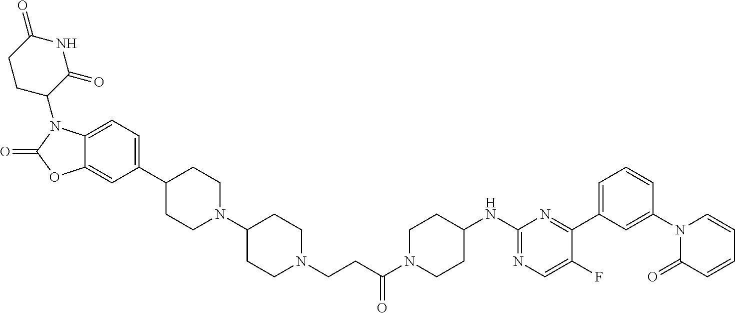 [SMILES: O=C1CCC(n2c(=O)oc3cc(C4CCN(C5CCN(CCC(=O)N6CCC(Nc7ncc(F)c(-c8cccc(-n9ccccc9=O)c8)n7)CC6)CC5)CC4)ccc32)C(=O)N1] |
| | 3-(6-(1′-(3-(4-((5-fluoro-4-(3-(2-oxopyridin-1(2H)-yl)phenyl)pyrimidin-2-yl)amino)piperidin-1-yl)-3-oxopropyl)-[1,4′- |
| | bipiperidin]-4-yl)-2-oxobenzo[d]oxazol-3(2H)-yl)piperidine-2,6-dione |
| 354 | 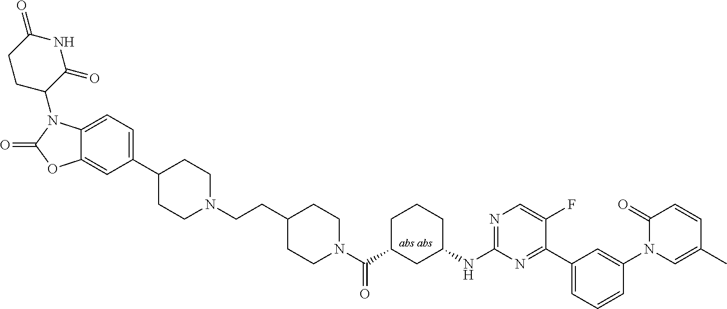 [SMILES: Cc1ccc(=O)n(-c2cccc(-c3nc(N[C@H]4CCC[C@@H](C(=O)N5CCC(CCN6CCC(c7ccc8c(c7)oc(=O)n8C7CCC(=O)NC7=O)CC6)CC5)C4)ncc3F)c2)c1] |
| | 3-(6-(1-(2-(1-((1R,3S)-3-((5-fluoro-4-(3-(5-methyl-2-oxopyridin-1(2H)-yl)phenyl)pyrimidin-2-yl)amino)cyclohexane-1- |
| | carbonyl)piperidin-4-yl)ethyl)piperidin-4-yl)-2-oxobenzo[d]oxazol-3(2H)-yl)piperidine-2,6-dione |
| 355 | 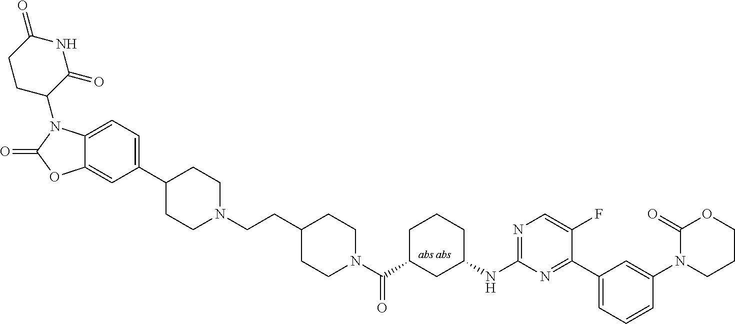 [SMILES: O=C1CCC(n2c(=O)oc3cc(C4CCN(CCC5CCN(C(=O)[C@@H]6CCC[C@H](Nc7ncc(F)c(-c8cccc(N9CCCOC9=O)c8)n7)C6)CC5)CC4)ccc32)C(=O)N1] |
| | 3-(6-(1-(2-(1-((1R,3S)-3-((5-fluoro-4-(3-(2-oxo-1,3-oxazinan-3-yl)phenyl)pyrimidin-2-yl)amino)cyclohexane-1- |
| | carbonyl)piperidin-4-yl)ethyl)piperidin-4-yl)-2-oxobenzo[d]oxazol-3(2H)-yl)piperidine-2,6-dione |
| 356 | 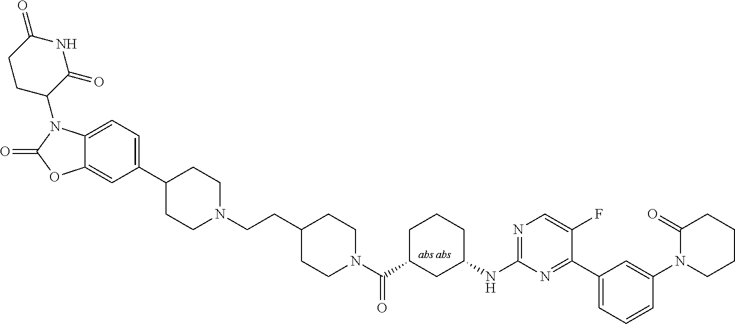 [SMILES: O=C1CCC(n2c(=O)oc3cc(C4CCN(CCC5CCN(C(=O)[C@@H]6CCC[C@H](Nc7ncc(F)c(-c8cccc(N9CCCCC9=O)c8)n7)C6)CC5)CC4)ccc32)C(=O)N1] |
| | 3-(6-(1-(2-(1-((1R,3S)-3-((5-fluoro-4-(3-(2-oxopiperidin-1-yl)phenyl)pyrimidin-2-yl)amino)cyclohexane-1-carbonyl)- |
| | piperidin-4-yl)ethyl)piperidin-4-yl)-2-oxobenzo[d]oxazol-3(2H)-yl)piperidine-2,6-dione |
| 357 | 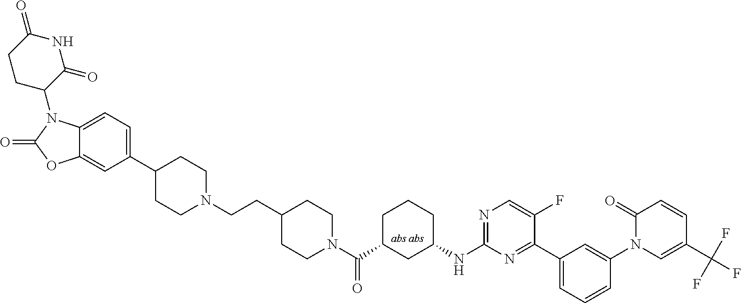 [SMILES: O=C1CCC(n2c(=O)oc3cc(C4CCN(CCC5CCN(C(=O)[C@@H]6CCC[C@H](Nc7ncc(F)c(-c8cccc(-n9cc(C(F)(F)F)ccc9=O)c8)n7)C6)CC5)CC4)ccc32)C(=O)N1] |
| | 3-(6-(1-(2-(1-((1R,3S)-3-((5-fluoro-4-(3-(2-oxo-5-(trifluoromethyl)pyridin-1(2H)-yl)phenyl)pyrimidin-2-yl)amino)- |
| | cyclohexane-1-carbonyl)piperidin-4-yl)ethyl)piperidin-4-yl)-2-oxobenzo[d]oxazol-3(2H)-yl)piperidine-2,6-dione |
| 358 | 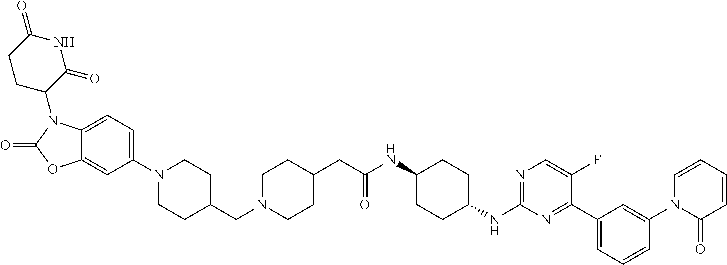 [SMILES: O=C1CCC(n2c(=O)oc3cc(N4CCC(CN5CCC(CC(=O)N[C@H]6CC[C@H](Nc7ncc(F)c(-c8cccc(-n9ccccc9=O)c8)n7)CC6)CC5)CC4)ccc32)C(=O)N1] |
| | trans-2-(1-((1-(3-(2,6-dioxopiperidin-3-yl)-2-oxo-2,3-dihydrobenzo[d]oxazol-6-yl)piperidin-4-yl)methyl)piperidin-4- |
| | yl)-N-(4-((5-fluoro-4-(3-(2-oxopyridin-1(2H)-yl)phenyl)pyrimidin-2-yl)amino)cyclohexyl)acetamide |
| 359 | 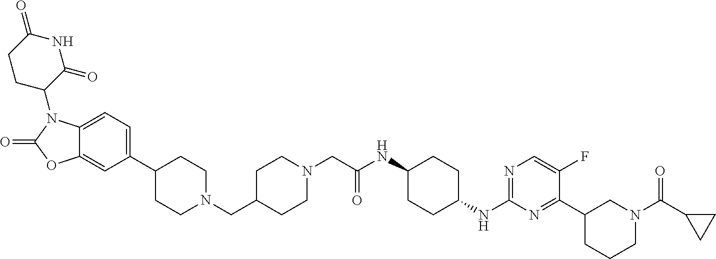 [SMILES: O=C1CCC(n2c(=O)oc3cc(C4CCN(CC5CCN(CC(=O)N[C@H]6CC[C@H](Nc7ncc(F)c(C8CCCN(C(=O)C9CC9)C8)n7)CC6)CC5)CC4)ccc32)C(=O)N1] |
| | trans-N-(4-((4-(1-(cyclopropanecarbonyl)piperidin-3-yl)-5-fluoropyrimidin-2-yl)amino)cyclohexyl)-2-(4-((4-(3-(2,6- |
| | dioxopiperidin-3-yl)-2-oxo-2,3-dihydrobenzo[d]oxazol-6-yl)piperidin-1-yl)methyl)piperidin-1-yl)acetamide |
| 360 | 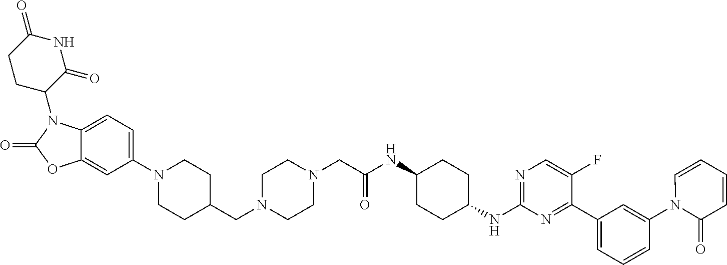 [SMILES: O=C1CCC(n2c(=O)oc3cc(N4CCC(CN5CCN(CC(=O)N[C@H]6CC[C@H](Nc7ncc(F)c(-c8cccc(-n9ccccc9=O)c8)n7)CC6)CC5)CC4)ccc32)C(=O)N1] |
| | 2-(4-((1-(3-(2,6-dioxopiperidin-3-yl)-2-oxo-2,3-dihydrobenzo[d]oxazol-6-yl)piperidin-4-yl)methyl)piperazin-1-yl)-N- |
| | (trans-4-((5-fluoro-4-(3-(2-oxopyridin-1(2H)-yl)phenyl)pyrimidin-2-yl)amino)cyclohexyl)acetamide |
| 361 | 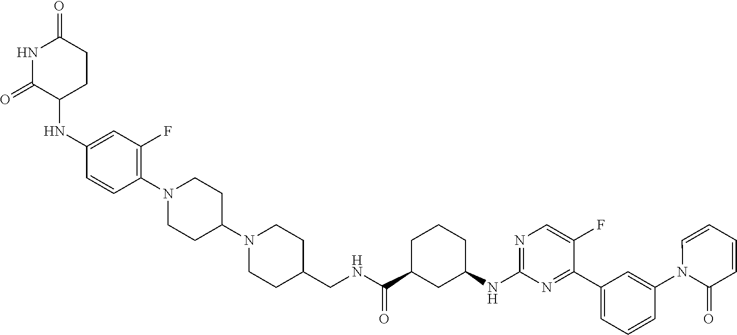 [SMILES: O=C1CCC(Nc2ccc(N3CCC(N4CCC(CNC(=O)[C@H]5CCC[C@@H](Nc6ncc(F)c(-c7cccc(-n8ccccc8=O)c7)n6)C5)CC4)CC3)c(F)c2)C(=O)N1] |
| | cis-N-((1′-(4-((2,6-dioxopiperidin-3-yl)amino)-2-fluorophenyl)-[1,4′-bipiperidin]-4-yl)methyl)-3-((5-fluoro-4-(3-(2- |
| | oxopyridin-1(2H)-yl)phenyl)pyrimidin-2-yl)amino)cyclohexane-1-carboxamide |
| 362 | 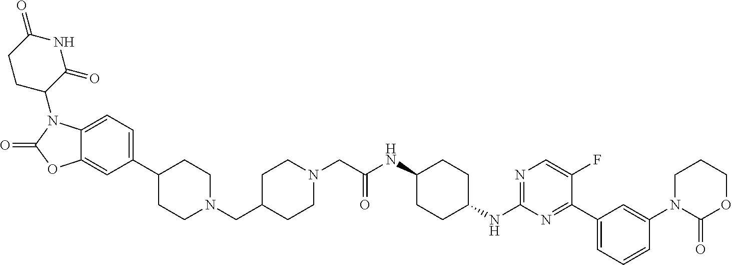 [SMILES: O=C1CCC(n2c(=O)oc3cc(C4CCN(CC5CCN(CC(=O)N[C@H]6CC[C@H](Nc7ncc(F)c(-c8cccc(N9CCCOC9=O)c8)n7)CC6)CC5)CC4)ccc32)C(=O)N1] |
| | trans-2-(4-((4-(3-(2,6-dioxopiperidin-3-yl)-2-oxo-2,3-dihydrobenzo[d]oxazol-6-yl)piperidin-1-yl)methyl)piperidin-1- |
| | yl)-N-(4-((5-fluoro-4-(3-(2-oxo-1,3-oxazinan-3-yl)phenyl)pyrimidin-2-yl)amino)cyclohexyl)acetamide |
| 363 | 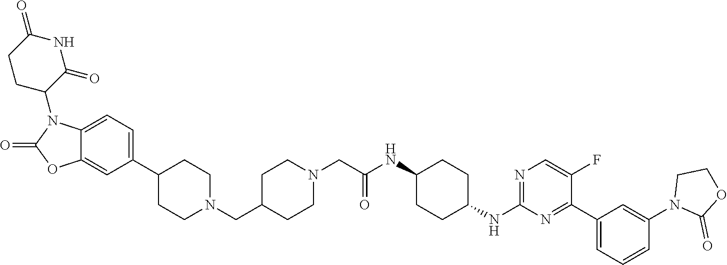 [SMILES: O=C1CCC(n2c(=O)oc3cc(C4CCN(CC5CCN(CC(=O)N[C@H]6CC[C@H](Nc7ncc(F)c(-c8cccc(N9CCOC9=O)c8)n7)CC6)CC5)CC4)ccc32)C(=O)N1] |
| | trans-2-[4-[[4-[3-(2,6-dioxo-3-piperidyl)-2-oxo-1,3-benzoxazol-6-yl]-1-piperidyl]methyl]-1-piperidyl]-N-[4-[[5-fluoro- |
| | 4-[3-(2-oxooxazolidin-3-yl)phenyl]pyrimidin-2-yl]amino]cyclohexyl]acetamide |
| 364 | 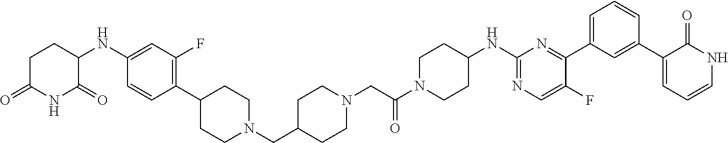 [SMILES: O=C1CCC(Nc2ccc(C3CCN(CC4CCN(CC(=O)N5CCC(Nc6ncc(F)c(-c7cccc(-c8ccc[nH]c8=O)c7)n6)CC5)CC4)CC3)c(F)c2)C(=O)N1] |
| | 3-((3-fluoro-4-(1-((1-(2-(4-((5-fluoro-4-(3-(2-oxo-1,2-dihydropyridin-3-yl)phenyl)pyrimidin-2-yl)amino)piperidin-1- |
| | yl)-2-oxoethyl)piperidin-4-yl)methyl)piperidin-4-yl)phenyl)amino)piperidine-2,6-dione |
| 365 | 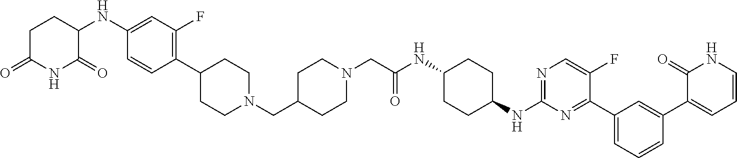 [SMILES: O=C1CCC(Nc2ccc(C3CCN(CC4CCN(CC(=O)N[C@H]5CC[C@H](Nc6ncc(F)c(-c7cccc(-c8ccc[nH]c8=O)c7)n6)CC5)CC4)CC3)c(F)c2)C(=O)N1] |
| | trans-2-(4-((4-(4-((2,6-dioxopiperidin-3-yl)amino)-2-fluorophenyl)piperidin-1-yl)methyl)piperidin-1-yl)-N-(4-((5-fluoro- |
| | 4-(3-(2-oxo-1,2-dihydropyridin-3-yl)phenyl)pyrimidin-2-yl)amino)cyclohexyl)acetamide |
| 366 | 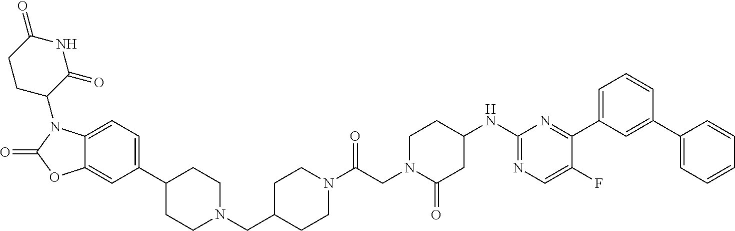 [SMILES: O=C1CCC(n2c(=O)oc3cc(C4CCN(CC5CCN(C(=O)CN6CCC(Nc7ncc(F)c(-c8cccc(-c9ccccc9)c8)n7)CC6=O)CC5)CC4)ccc32)C(=O)N1] |
| | 3-(6-(1-((1-(2-(4-((4-([1,1′-biphenyl]-3-yl)-5-fluoropyrimidin-2-yl)amino)-2-oxopiperidin-1-yl)acetyl)piperidin-4-yl)- |
| | methyl)piperidin-4-yl)-2-oxobenzo[d]oxazol-3(2H)-yl)piperidine-2,6-dione |
| 367 | 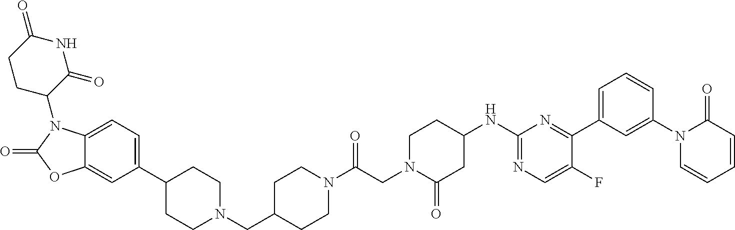 [SMILES: O=C1CCC(n2c(=O)oc3cc(C4CCN(CC5CCN(C(=O)CN6CCC(Nc7ncc(F)c(-c8cccc(-n9ccccc9=O)c8)n7)CC6=O)CC5)CC4)ccc32)C(=O)N1] |
| | 3-(6-(1-((1-(2-(4-((5-fluoro-4-(3-(2-oxopyridin-1(2H)-yl)phenyl)pyrimidin-2-yl)amino)-2-oxopiperidin-1-yl)acetyl)- |
| | piperidin-4-yl)methyl)piperidin-4-yl)-2-oxobenzo[d]oxazol-3(2H)-yl)piperidine-2,6-dione |
| 368 | 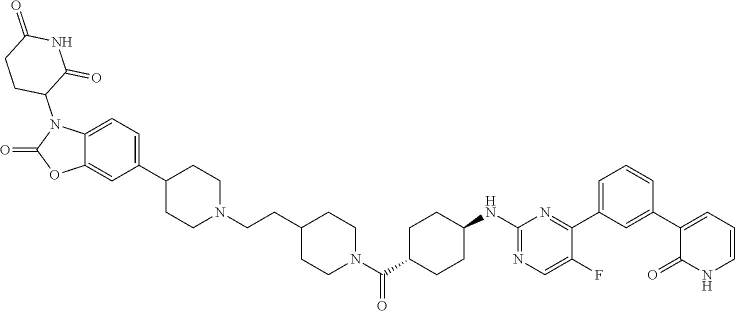 [SMILES: O=C1CCC(n2c(=O)oc3cc(C4CCN(CCC5CCN(C(=O)[C@H]6CC[C@H](Nc7ncc(F)c(-c8cccc(-c9ccc[nH]c9=O)c8)n7)CC6)CC5)CC4)ccc32)C(=O)N1] |
| | 3-(6-(1-(2-(1-(trans-4-((5-fluoro-4-(3-(2-oxo-1,2-dihydropyridin-3-yl)phenyl)pyrimidin-2-yl)amino)cyclohexane-1- |
| | carbonyl)piperidin-4-yl)ethyl)piperidin-4-yl)-2-oxobenzo[d]oxazol-3(2H)-yl)piperidine-2,6-dione |
| 369 | 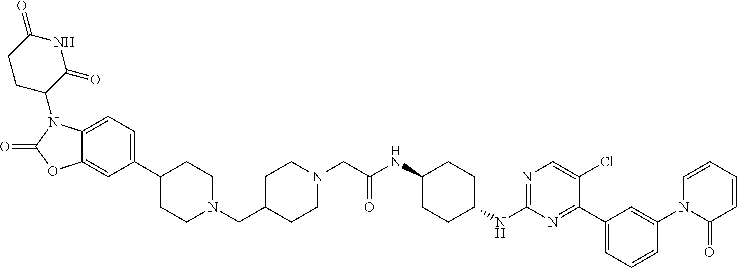 [SMILES: O=C1CCC(n2c(=O)oc3cc(C4CCN(CC5CCN(CC(=O)N[C@H]6CC[C@H](Nc7ncc(Cl)c(-c8cccc(-n9ccccc9=O)c8)n7)CC6)CC5)CC4)ccc32)C(=O)N1] |
| | trans-N-(4-((5-chloro-4-(3-(2-oxopyridin-1(2H)-yl)phenyl)pyrimidin-2-yl)amino)cyclohexyl)-2-(4-((4-(3-(2,6-dioxo- |
| | piperidin-3-yl)-2-oxo-2,3-dihydrobenzo[d]oxazol-6-yl)piperidin-1-yl)methyl)piperidin-1-yl)acetamide |
| 370 | 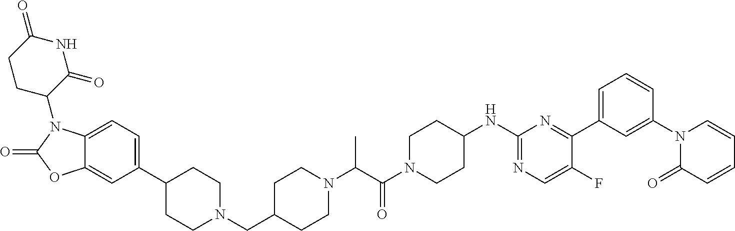 [SMILES: CC(C(=O)N1CCC(Nc2ncc(F)c(-c3cccc(-n4ccccc4=O)c3)n2)CC1)N1CCC(CN2CCC(c3ccc4c(c3)oc(=O)n4C3CCC(=O)NC3=O)CC2)CC1] |
| | 3-(6-(1-((1-(1-(4-((5-fluoro-4-(3-(2-oxopyridin-1(2H)-yl)phenyl)pyrimidin-2-yl)amino)piperidin-1-yl)-1-oxopropan-2- |
| | yl)piperidin-4-yl)methyl)piperidin-4-yl)-2-oxobenzo[d]oxazol-3(2H)-yl)piperidine-2,6-dione |
| 371 | 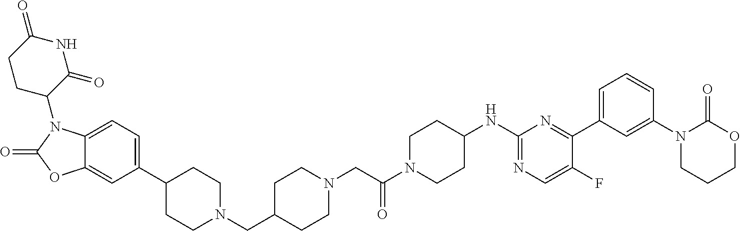 [SMILES: O=C1CCC(n2c(=O)oc3cc(C4CCN(CC5CCN(CC(=O)N6CCC(Nc7ncc(F)c(-c8cccc(N9CCCOC9=O)c8)n7)CC6)CC5)CC4)ccc32)C(=O)N1] |
| | 3-(6-(1-((1-(2-(4-((5-fluoro-4-(3-(2-oxo-1,3-oxazinan-3-yl)phenyl)pyrimidin-2-yl)amino)piperidin-1-yl)-2-oxoethyl)- |
| | piperidin-4-yl)methyl)piperidin-4-yl)-2-oxobenzo[d]oxazol-3(2H)-yl)piperidine-2,6-dione |
| 372 | 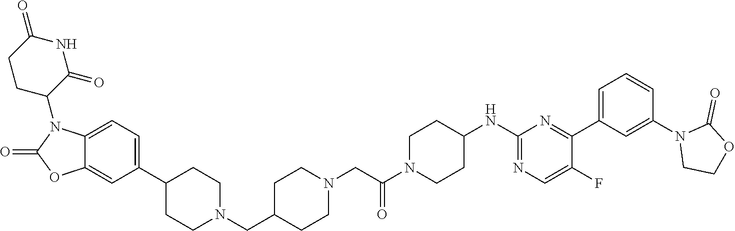 [SMILES: O=C1CCC(n2c(=O)oc3cc(C4CCN(CC5CCN(CC(=O)N6CCC(Nc7ncc(F)c(-c8cccc(N9CCOC9=O)c8)n7)CC6)CC5)CC4)ccc32)C(=O)N1] |
| | 3-(6-(1-((1-(2-(4-((5-fluoro-4-(3-(2-oxooxazolidin-3-yl)phenyl)pyrimidin-2-yl)amino)piperidin-1-yl)-2-oxoethyl)- |
| | piperidin-4-yl)methyl)piperidin-4-yl)-2-oxobenzo[d]oxazol-3(2H)-yl)piperidine-2,6-dione |
| 373 | 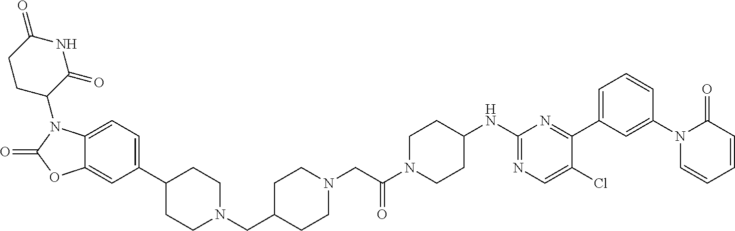 [SMILES: O=C1CCC(n2c(=O)oc3cc(C4CCN(CC5CCN(CC(=O)N6CCC(Nc7ncc(Cl)c(-c8cccc(-n9ccccc9=O)c8)n7)CC6)CC5)CC4)ccc32)C(=O)N1] |
| | 3-(6-(1-((1-(2-(4-((5-chloro-4-(3-(2-oxopyridin-1(2H)-yl)phenyl)pyrimidin-2-yl)amino)piperidin-1-yl)-2-oxoethyl)- |
| | piperidin-4-yl)methyl)piperidin-4-yl)-2-oxobenzo[d]oxazol-3(2H)-yl)piperidine-2,6-dione |
| 374 | 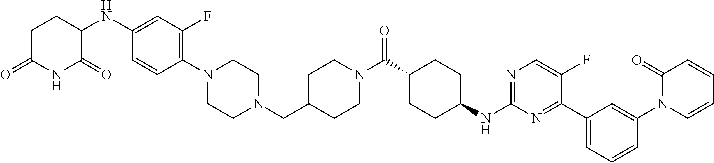 [SMILES: O=C1CCC(Nc2ccc(N3CCN(CC4CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-3-((3-fluoro-4-(4-((1-(4-((5-fluoro-4-(3-(2-oxopyridin-1(2H)-yl)phenyl)pyrimidin-2-yl)amino)cyclohexane- |
| | 1-carbonyl)piperidin-4-yl)methyl)piperazin-1-yl)phenyl)amino)piperidine-2,6-dione |
| 375 | 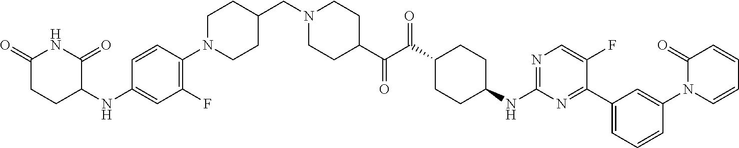 [SMILES: O=C1CCC(Nc2ccc(N3CCC(CN4CCC(C(=O)C(=O)[C@H]5CC[C@H](Nc6ncc(F)c(-c7cccc(-n8ccccc8=O)c7)n6)CC5)CC4)CC3)c(F)c2)C(=O)N1] |
| | trans-N-(1-((1-(4-((2,6-dioxopiperidin-3-yl)amino)-2-fluorophenyl)piperidin-4-yl)methyl)piperidin-4-yl)-4-((5-fluoro-4- |
| | (3-(2-oxopyridin-1(2H)-yl)phenyl)pyrimidin-2-yl)amino)cyclohexane-1-carboxamide |
| 376 | 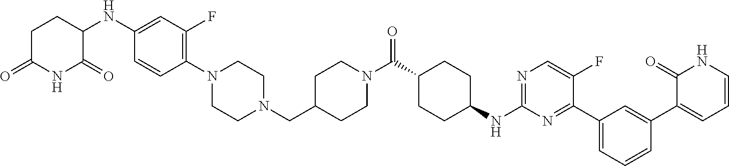 [SMILES: O=C1CCC(Nc2ccc(N3CCN(CC4CCN(C(=O)[C@H]5CC[C@H](Nc6ncc(F)c(-c7cccc(-c8ccc[nH]c8=O)c7)n6)CC5)CC4)CC3)c(F)c2)C(=O)N1] |
| | trans-3-((3-fluoro-4-(4-((1-(4-((5-fluoro-4-(3-(2-oxo-1,2-dihydropyridin-3-yl)phenyl)pyrimidin-2-yl)amino)cyclohexane- |
| | 1-carbonyl)piperidin-4-yl)methyl)piperazin-1-yl)phenyl)amino)piperidine-2,6-dione |
| 377 | 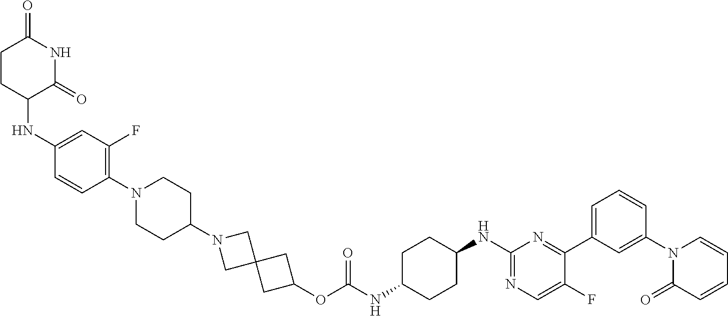 [SMILES: O=C1CCC(Nc2ccc(N3CCC(N4CC5(CC(OC(=O)N[C@H]6CC[C@H](Nc7ncc(F)c(-c8cccc(-n9ccccc9=O)c8)n7)CC6)C5)C4)CC3)c(F)c2)C(=O)N1] |
| | trans-2-(1-(4-((2,6-dioxopiperidin-3-yl)amino)-2-fluorophenyl)piperidin-4-yl)-2-azaspiro[3.3]heptan-6-yl(4-((5-fluoro- |
| | 4-(3-(2-oxopyridin-1(2H)-yl)phenyl)pyrimidin-2-yl)amino)cyclohexyl)carbamate |
| 378 | 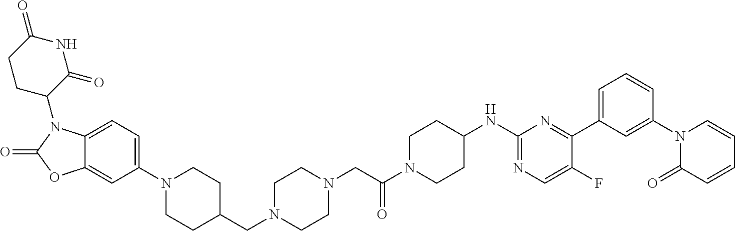 [SMILES: O=C1CCC(n2c(=O)oc3cc(N4CCC(CN5CCN(CC(=O)N6CCC(Nc7ncc(F)c(-c8cccc(-n9ccccc9=O)c8)n7)CC6)CC5)CC4)ccc32)C(=O)N1] |
| | 3-(6-(4-((4-(2-(4-((5-fluoro-4-(3-(2-oxopyridin-1(2H)-yl)phenyl)pyrimidin-2-yl)amino)piperidin-1-yl)-2-oxoethyl)- |
| | piperazin-1-yl)methyl)piperidin-1-yl)-2-oxobenzo[d]oxazol-3(2H)-yl)piperidine-2,6-dione |
| 379 | 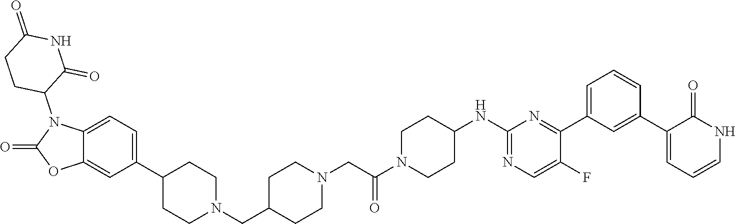 [SMILES: O=C1CCC(n2c(=O)oc3cc(C4CCN(CC5CCN(CC(=O)N6CCC(Nc7ncc(F)c(-c8cccc(-c9ccc[nH]c9=O)c8)n7)CC6)CC5)CC4)ccc32)C(=O)N1] |
| | 3-[6-[1-[[1-[2-[4-[5-fluoro-4-[3-(2-oxo-1H-pyridin-3-yl)phenyl]pyrimidin-2-yl]amino]-1-piperidyl]-2-oxo-ethyl]-4- |
| | piperidyl]methyl]-4-piperidyl]-2-oxo-1,3-benzoxazol-3-yl]piperidine-2,6-dione |
| 380 | 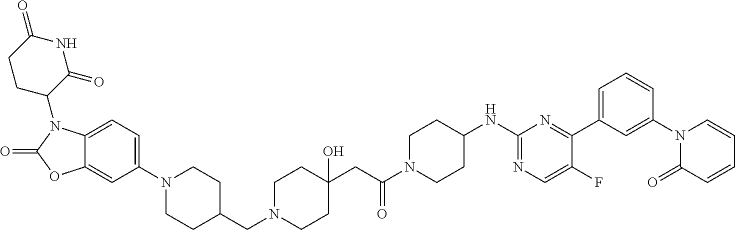 [SMILES: O=C1CCC(n2c(=O)oc3cc(N4CCC(CN5CCC(O)(CC(=O)N6CCC(Nc7ncc(F)c(-c8cccc(-n9ccccc9=O)c8)n7)CC6)CC5)CC4)ccc32)C(=O)N1] |
| | 3-(6-(4-((4-(2-(4-((5-fluoro-4-(3-(2-oxopyridin-1(2H)-yl)phenyl)pyrimidin-2-yl)amino)piperidin-1-yl)-2-oxoethyl)- |
| | 4-hydroxypiperidin-1-yl)methyl)piperidin-1-yl)-2-oxobenzo[d]oxazol-3(2H)-yl)piperidine-2,6-dione |
| 381 | 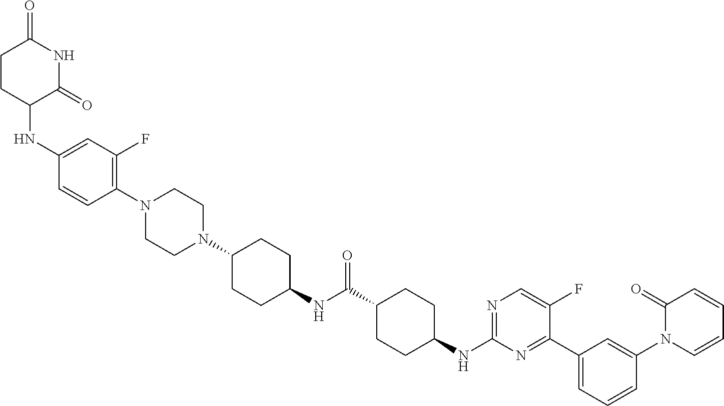 [SMILES: O=C1CCC(Nc2ccc(N3CCN([C@H]4CC[C@H](NC(=O)[C@H]5CC[C@H](Nc6ncc(F)c(-c7cccc(-n8ccccc8=O)c7)n6)CC5)CC4)CC3)c(F)c2)C(=O)N1] |
| | trans-(1r,4R)-N-((1r,4R)-4-(4-(4-((2,6-dioxopiperidin-3-yl)amino)-2-fluorophenyl)piperazin-1-yl)cyclohexyl)-4-((5- |
| | fluoro-4-(3-(2-oxopyridin-1(2H)-yl)phenyl)pyrimidin-2-yl)amino)cyclohexane-1-carboxamide |
| 382 | 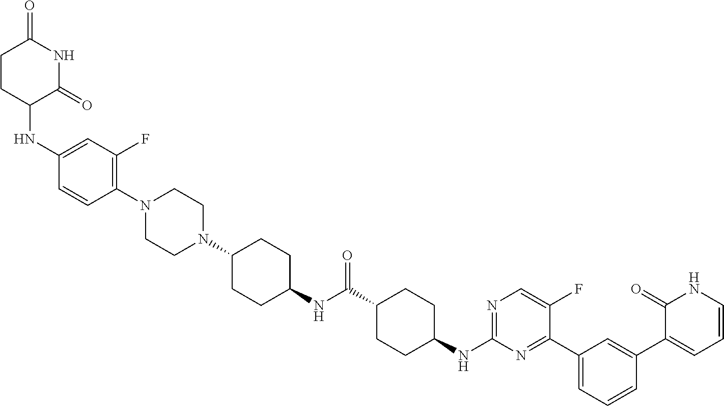 [SMILES: O=C1CCC(Nc2ccc(N3CCN([C@H]4CC[C@H](NC(=O)[C@H]5CC[C@H](Nc6ncc(F)c(-c7cccc(-c8ccc[nH]c8=O)c7)n6)CC5)CC4)CC3)c(F)c2)C(=O)N1] |
| | trans-N-(trans-4-(4-(4-((2,6-dioxopiperidin-3-yl)amino)-2-fluorophenyl)piperazin-1-yl)cyclohexyl)-4-((5-fluoro- |
| | 4-(3-(2-oxo-1,2-dihydropyridin-3-yl)phenyl)pyrimidin-2-yl)amino)cyclohexane-1-carboxamide |
| 383 | 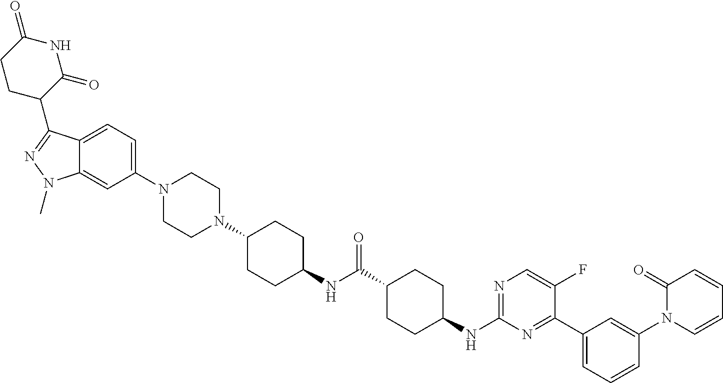 [SMILES: Cn1nc(C2CCC(=O)NC2=O)c2ccc(N3CCN([C@H]4CC[C@H](NC(=O)[C@H]5CC[C@H](Nc6ncc(F)c(-c7cccc(-n8ccccc8=O)c7)n6)CC5)CC4)CC3)cc21] |
| | trans-N-(trans-4-(4-(3-(2,6-dioxopiperidin-3-yl)-1-methyl-1H-indazol-6-yl)piperazin-1-yl)cyclohexyl)-4-((5-fluoro-4- |
| | (3-(2-oxopyridin-1(2H)-yl)phenyl)pyrimidin-2-yl)amino)cyclohexane-1-carboxamide |
| 384 | 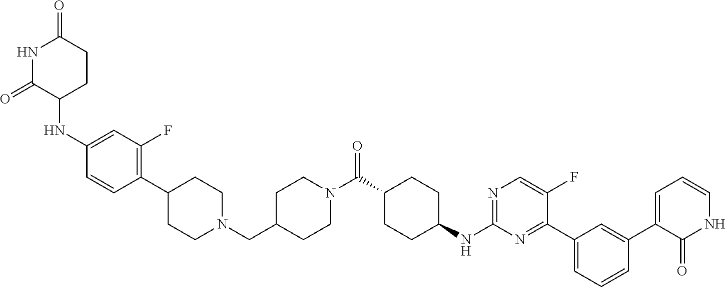 [SMILES: O=C1CCC(Nc2ccc(C3CCN(CC4CCN(C(=O)[C@H]5CC[C@H](Nc6ncc(F)c(-c7cccc(-c8ccc[nH]c8=O)c7)n6)CC5)CC4)CC3)c(F)c2)C(=O)N1] |
| | trans-3-((3-fluoro-4-(1-((1-(4-((5-fluoro-4-(3-(2-oxo-1,2-dihydropyridin-3-yl)phenyl)pyrimidin-2-yl)amino)cyclohexane- |
| | 1-carbonyl)piperidin-4-yl)methyl)piperidin-4-yl)phenyl)amino)piperidine-2,6-dione |
| 385 | 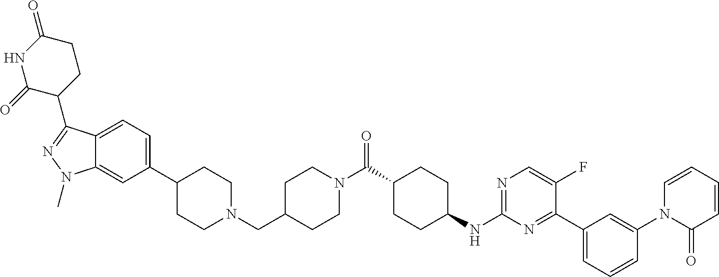 [SMILES: Cn1nc(C2CCC(=O)NC2=O)c2ccc(C3CCN(CC4CCN(C(=O)[C@H]5CC[C@H](Nc6ncc(F)c(-c7cccc(-n8ccccc8=O)c7)n6)CC5)CC4)CC3)cc21] |
| | trans-3-(6-(1-((1-(4-((5-fluoro-4-(3-(2-oxopyridin-1(2H)-yl)phenyl)pyrimidin-2-yl)amino)cyclohexane-1-carbonyl)- |
| | piperidin-4-yl)methyl)piperidin-4-yl)-1-methyl-1H-indazol-3-yl)piperidine-2,6-dione |
| 386 | 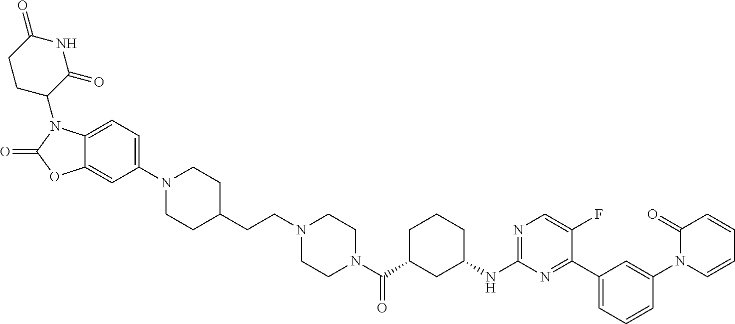 [SMILES: O=C1CCC(n2c(=O)oc3cc(N4CCC(CCN5CCN(C(=O)[C@@H]6CCC[C@H](Nc7ncc(F)c(-c8cccc(-n9ccccc9=O)c8)n7)C6)CC5)CC4)ccc32)C(=O)N1] |
| | 3-(6-(4-(2-(4-(cis-3-((5-fluoro-4-(3-(2-oxopyridin-1(2H)-yl)phenyl)pyrimidin-2-yl)amino)cyclohexane-1-carbonyl)- |
| | piperazin-1-yl)ethyl)piperidin-1-yl)-2-oxobenzo[d]oxazol-3(2H)-yl)piperidine-2,6-dione |
| 387 | 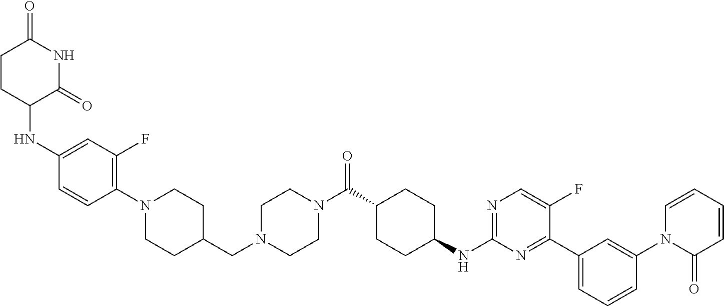 [SMILES: O=C1CCC(Nc2ccc(N3CCC(CN4CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-3-((3-fluoro-4-(4-((4-(4-((5-fluoro-4-(3-(2-oxopyridin-1(2H)-yl)phenyl)pyrimidin-2-yl)amino)cyclohexane- |
| | 1-carbonyl)piperazin-1-yl)methyl)piperidin-1-yl)phenyl)amino)piperidine-2,6-dione |
| 388 | 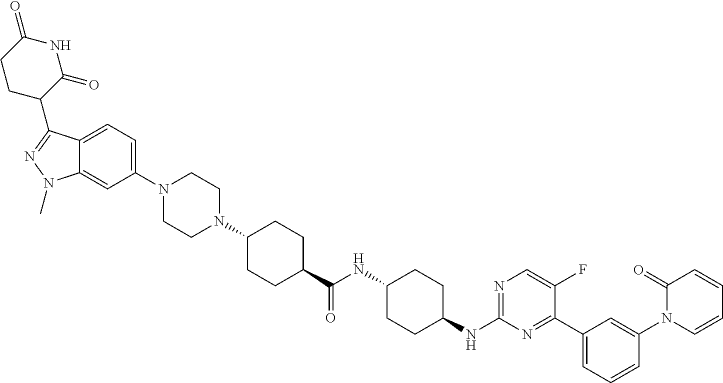 [SMILES: Cn1nc(C2CCC(=O)NC2=O)c2ccc(N3CCN([C@H]4CC[C@H](C(=O)N[C@H]5CC[C@H](Nc6ncc(F)c(-c7cccc(-n8ccccc8=O)c7)n6)CC5)CC4)CC3)cc21] |
| | trans-4-(4-(3-(2,6-dioxopiperidin-3-yl)-1-methyl-1H-indazol-6-yl)piperazin-1-yl)-N-(trans-4-((5-fluoro-4-(3-(2- |
| | oxopyridin-1(2H)-yl)phenyl)pyrimidin-2-yl)amino)cyclohexyl)cyclohexane-1-carboxamide |
| 389 | 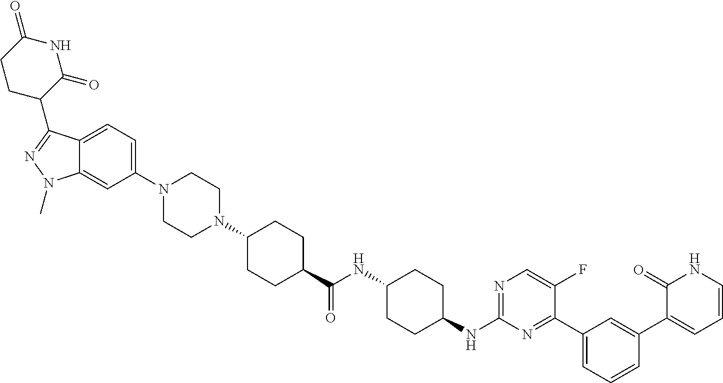 [SMILES: Cn1nc(C2CCC(=O)NC2=O)c2ccc(N3CCN([C@H]4CC[C@H](C(=O)N[C@H]5CC[C@H](Nc6ncc(F)c(-c7cccc(-c8ccc[nH]c8=O)c7)n6)CC5)CC4)CC3)cc21] |
| | trans-(1r,4R)-4-(4-(3-(2,6-dioxopiperidin-3-yl)-1-methyl-1H-indazol-6-yl)piperazin-1-yl)-N-((1r,4R)-4-((5-fluoro-4-(3- |
| | (2-oxo-1,2-dihydropyridin-3-yl)phenyl)pyrimidin-2-yl)amino)cyclohexyl)cyclohexane-1-carboxamide |
| 390 | 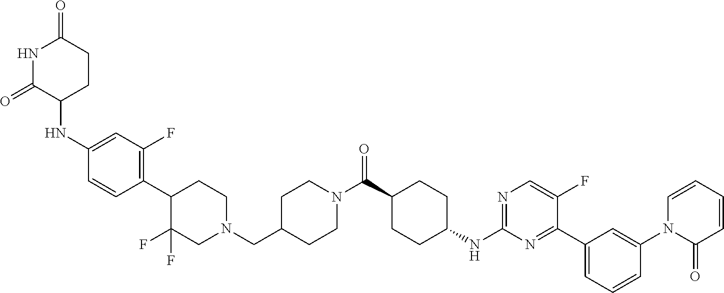 [SMILES: O=C1CCC(Nc2ccc(C3CCN(CC4CCN(C(=O)[C@H]5CC[C@H](Nc6ncc(F)c(-c7cccc(-n8ccccc8=O)c7)n6)CC5)CC4)CC3(F)F)c(F)c2)C(=O)N1] |
| | trans-3-[4-[3,3-difluoro-1-[[1-[4-[[5-fluoro-4-[3-(2-oxo-1-pyridyl)phenyl]pyrimidin-2-yl]amino]cyclohexanecarbonyl]- |
| | 4-piperidyl]methyl]-4-piperidyl]-3-fluoro-anilino]piperidine-2,6-dione |
| 391 | 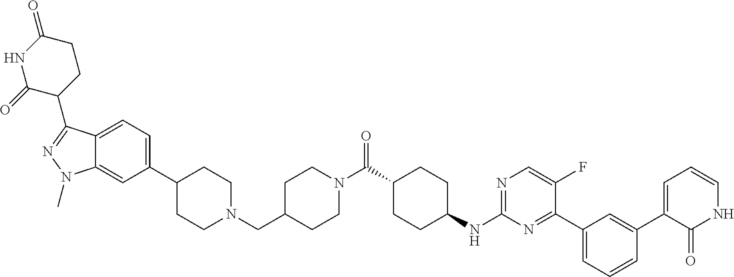 [SMILES: Cn1nc(C2CCC(=O)NC2=O)c2ccc(C3CCN(CC4CCN(C(=O)[C@H]5CC[C@H](Nc6ncc(F)c(-c7cccc(-c8ccc[nH]c8=O)c7)n6)CC5)CC4)CC3)cc21] |
| | trans-3-(6-(1-((1-(4-((5-fluoro-4-(3-(2-oxo-1,2-dihydropyridin-3-yl)phenyl)pyrimidin-2-yl)amino)cyclohexane-1- |
| | carbonyl)piperidin-4-yl)methyl)piperidin-4-yl)-1-methyl-1H-indazol-3-yl)piperidine-2,6-dione |
| 392 | 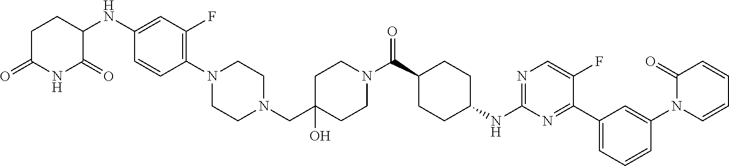 [SMILES: O=C1CCC(Nc2ccc(N3CCN(CC4(O)CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-3-((3-fluoro-4-(4-((1-(4-((5-fluoro-4-(3-(2-oxopyridin-1(2H)-yl)phenyl)pyrimidin-2-yl)amino)cyclohexane-1- |
| | carbonyl)-4-hydroxypiperidin-4-yl)methyl)piperazin-1-yl)phenyl)amino)piperidine-2,6-dione |
| 393 | 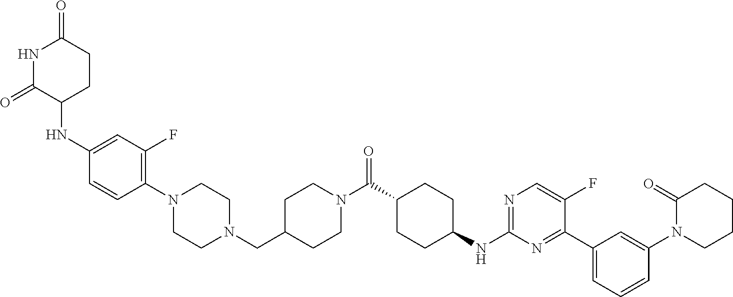 [SMILES: O=C1CCC(Nc2ccc(N3CCN(CC4CCN(C(=O)[C@H]5CC[C@H](Nc6ncc(F)c(-c7cccc(N8CCCCC8=O)c7)n6)CC5)CC4)CC3)c(F)c2)C(=O)N1] |
| | trans-3-[3-fluoro-4-[4-[[1-[4-[5-fluoro-4-[3-(2-oxo-1-piperidyl)phenyl]pyrimidin-2-yl]amino]cyclohexanecarbonyl]-4- |
| | piperidyl]methyl]piperazin-1-yl]anilino]piperidine-2,6-dione |
| 394 | 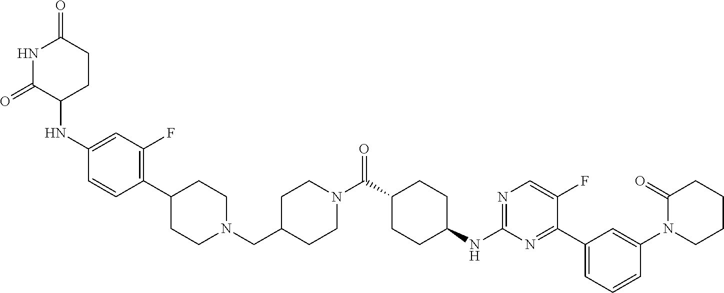 [SMILES: O=C1CCC(Nc2ccc(C3CCN(CC4CCN(C(=O)[C@H]5CC[C@H](Nc6ncc(F)c(-c7cccc(N8CCCCC8=O)c7)n6)CC5)CC4)CC3)c(F)c2)C(=O)N1] |
| | trans-3-((3-fluoro-4-(1-((1-(4-((5-fluoro-4-(3-(2-oxopiperidin-1-yl)phenyl)pyrimidin-2-yl)amino)cyclohexane-1- |
| | carbonyl)piperidin-4-yl)methyl)piperidin-4-yl)phenyl)amino)piperidine-2,6-dione |
| 395 | 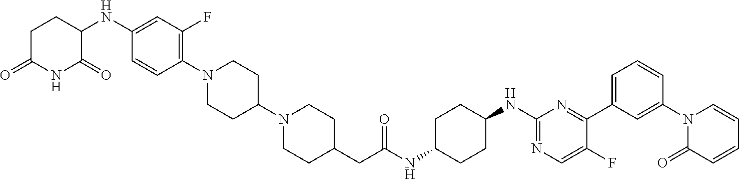 [SMILES: O=C1CCC(Nc2ccc(N3CCC(N4CCC(CC(=O)N[C@H]5CC[C@H](Nc6ncc(F)c(-c7cccc(-n8ccccc8=O)c7)n6)CC5)CC4)CC3)c(F)c2)C(=O)N1] |
| | trans-2-(1′-(4-((2,6-dioxopiperidin-3-yl)amino)-2-fluorophenyl)-[1,4′-bipiperidin]-4-yl)-N-((1r,4r)-4-((5-fluoro-4-(3- |
| | (2-oxopyridin-1(2H)-yl)phenyl)pyrimidin-2-yl)amino)cyclohexyl)acetamide |
| 396 | 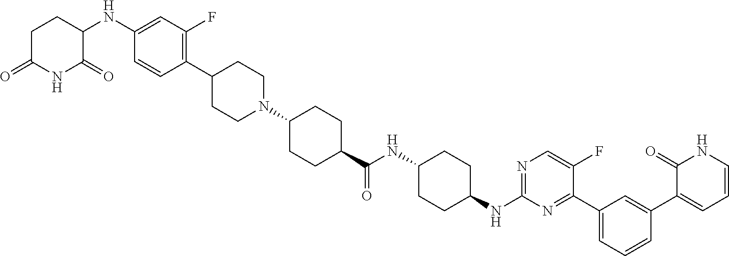 [SMILES: O=C1CCC(Nc2ccc(C3CCN([C@H]4CC[C@H](C(=O)N[C@H]5CC[C@H](Nc6ncc(F)c(-c7cccc(-c8ccc[nH]c8=O)c7)n6)CC5)CC4)CC3)c(F)c2)C(=O)N1] |
| | trans-4-(4-(4-((2,6-dioxopiperidin-3-yl)amino)-2-fluorophenyl)piperidin-1-yl)-N-(trans-4-((5-fluoro-4-(3-(2-oxo-1,2- |
| | dihydropyridin-3-yl)phenyl)pyrimidin-2-yl)amino)cyclohexyl)cyclohexane-1-carboxamide |
| 397 | 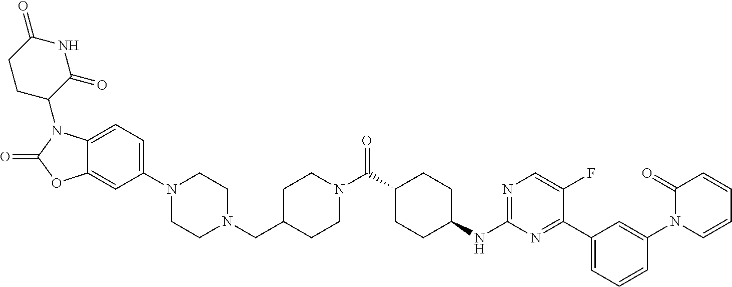 [SMILES: O=C1CCC(n2c(=O)oc3cc(N4CCN(CC5CCN(C(=O)[C@H]6CC[C@H](Nc7ncc(F)c(-c8cccc(-n9ccccc9=O)c8)n7)CC6)CC5)CC4)ccc32)C(=O)N1] |
| | trans-3-(6-(4-((1-(4-((5-fluoro-4-(3-(2-oxopyridin-1(2H)-yl)phenyl)pyrimidin-2-yl)amino)cyclohexane-1-carbonyl)- |
| | piperidin-4-yl)methyl)piperazin-1-yl)-2-oxobenzo[d]oxazol-3(2H)-yl)piperidine-2,6-dione |
| 398 | 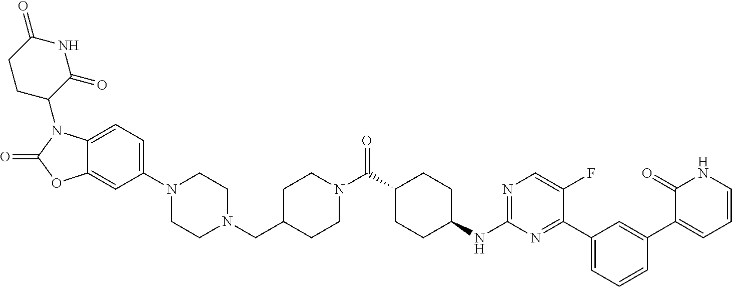 [SMILES: O=C1CCC(n2c(=O)oc3cc(N4CCN(CC5CCN(C(=O)[C@H]6CC[C@H](Nc7ncc(F)c(-c8cccc(-c9ccc[nH]c9=O)c8)n7)CC6)CC5)CC4)ccc32)C(=O)N1] |
| | trans-3-(6-(4-((1-(4-((5-fluoro-4-(3-(2-oxo-1,2-dihydropyridin-3-yl)phenyl)pyrimidin-2-yl)amino)cyclohexane-1- |
| | carbonyl)piperidin-4-yl)methyl)piperazin-1-yl)-2-oxobenzo[d]oxazol-3(2H)-yl)piperidine-2,6-dione |
| 399 | 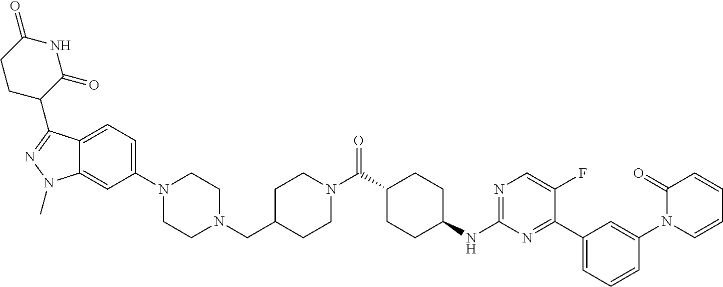 [SMILES: Cn1nc(C2CCC(=O)NC2=O)c2ccc(N3CCN(CC4CCN(C(=O)[C@H]5CC[C@H](Nc6ncc(F)c(-c7cccc(-n8ccccc8=O)c7)n6)CC5)CC4)CC3)cc21] |
| | 3-(6-(4-((1-(trans-4-((5-fluoro-4-(3-(2-oxopyridin-1(2H)-yl)phenyl)pyrimidin-2-yl)amino)cyclohexane-1-carbonyl)- |
| | piperidin-4-yl)methyl)piperazin-1-yl)-1-methyl-1H-indazol-3-yl)piperidine-2,6-dione |
| 400 | 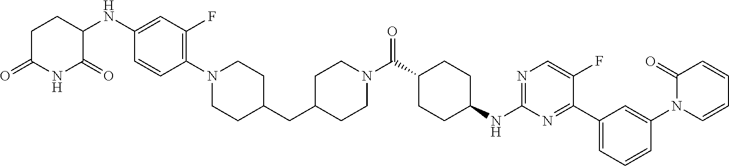 [SMILES: O=C1CCC(Nc2ccc(N3CCC(CC4CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-3-((3-fluoro-4-(4-((1-(4-((5-fluoro-4-(3-(2-oxopyridin-1(2H)-yl)phenyl)pyrimidin-2-yl)amino)cyclohexane-1- |
| | carbonyl)piperidin-4-yl)methyl)piperidin-1-yl)phenyl)amino)piperidine-2,6-dione |
| 401 | 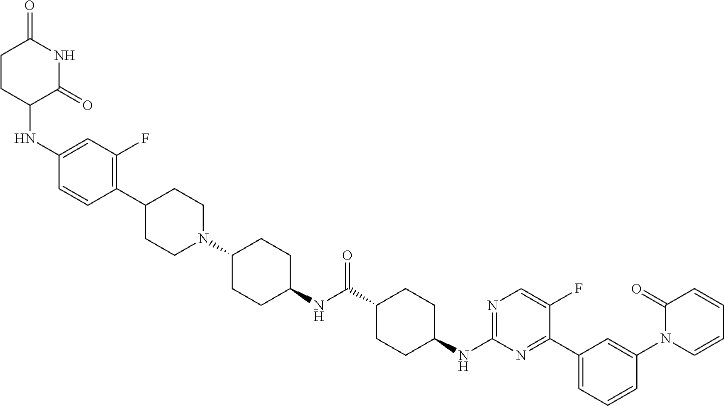 [SMILES: O=C1CCC(Nc2ccc(C3CCN([C@H]4CC[C@H](NC(=O)[C@H]5CC[C@H](Nc6ncc(F)c(-c7cccc(-n8ccccc8=O)c7)n6)CC5)CC4)CC3)c(F)c2)C(=O)N1] |
| | trans-(1r,4R)-N-((1r,4R)-4-(4-(4-((2,6-dioxopiperidin-3-yl)amino)-2-fluorophenyl)piperidin-1-yl)cyclohexyl)-4- |
| | ((5-fluoro-4-(3-(2-oxopyridin-1(2H)-yl)phenyl)pyrimidin-2-yl)amino)cyclohexane-1-carboxamide |
| 402 | 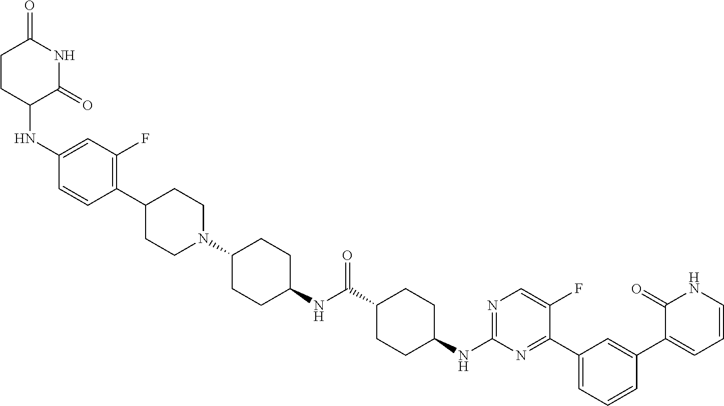 [SMILES: O=C1CCC(Nc2ccc(C3CCN([C@H]4CC[C@H](NC(=O)[C@H]5CC[C@H](Nc6ncc(F)c(-c7cccc(-c8ccc[nH]c8=O)c7)n6)CC5)CC4)CC3)c(F)c2)C(=O)N1] |
| | trans-(1r,4R)-N-((1r,4R)-4-(4-(4-((2,6-dioxopiperidin-3-yl)amino)-2-fluorophenyl)piperidin-1-yl)cyclohexyl)-4-((5- |
| | fluoro-4-(3-(2-oxo-1,2-dihydropyridin-3-yl)phenyl)pyrimidin-2-yl)amino)cyclohexane-1-carboxamide |
| 403 | 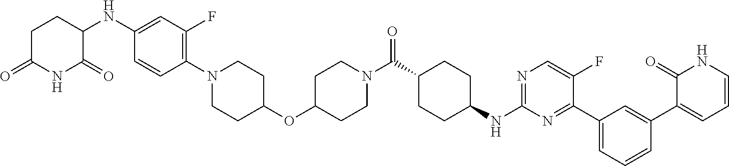 [SMILES: O=C1CCC(Nc2ccc(N3CCC(OC4CCN(C(=O)[C@H]5CC[C@H](Nc6ncc(F)c(-c7cccc(-c8ccc[nH]c8=O)c7)n6)CC5)CC4)CC3)c(F)c2)C(=O)N1] |
| | trans-3-((3-fluoro-4-(4-((1-(4-((5-fluoro-4-(3-(2-oxo-1,2-dihydropyridin-3-yl)phenyl)pyrimidin-2-yl)amino)cyclohexane- |
| | 1-carbonyl)piperidin-4-yl)oxy)piperidin-1-yl)phenyl)amino)piperidine-2,6-dione |
| 404 | 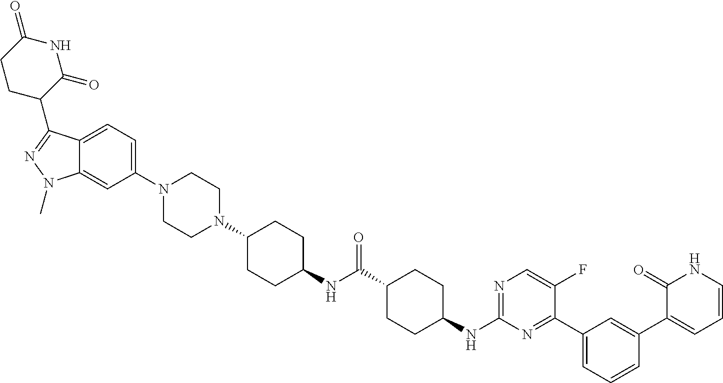 [SMILES: Cn1nc(C2CCC(=O)NC2=O)c2ccc(N3CCN([C@H]4CC[C@H](NC(=O)[C@H]5CC[C@H](Nc6ncc(F)c(-c7cccc(-c8ccc[nH]c8=O)c7)n6)CC5)CC4)CC3)cc21] |
| | trans-(1r,4R)-N-((1r,4R)-4-(4-(3-(2,6-dioxopiperidin-3-yl)-1-methyl-1H-indazol-6-yl)piperazin-1-yl)cyclohexyl)-4- |
| | ((5-fluoro-4-(3-(2-oxo-1,2-dihydropyridin-3-yl)phenyl)pyrimidin-2-yl)amino)cyclohexane-1-carboxamide |
| 405 | 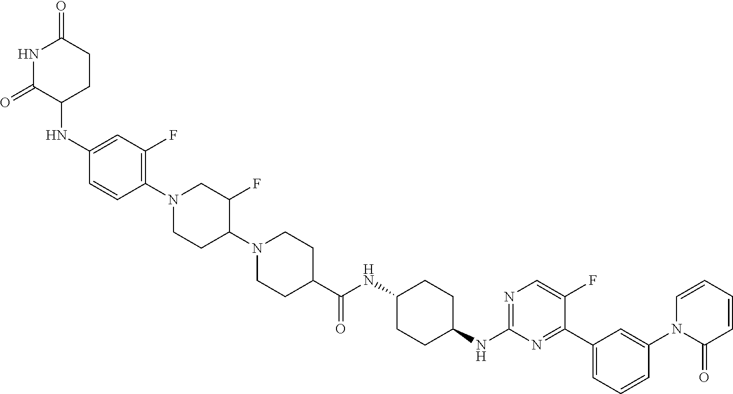 [SMILES: O=C1CCC(Nc2ccc(N3CCC(N4CCC(C(=O)N[C@H]5CC[C@H](Nc6ncc(F)c(-c7cccc(-n8ccccc8=O)c7)n6)CC5)CC4)C(F)C3)c(F)c2)C(=O)N1] |
| | 1′-(4-((2,6-dioxopiperidin-3-yl)amino)-2-fluorophenyl)-3′-fluoro-N-((1r,4r)-4-((5-fluoro-4-(3-(2-oxopyridin-1(2H)-yl)- |
| | phenyl)pyrimidin-2-yl)amino)cyclohexyl)-[1,4′-bipiperidine]-4-carboxamide |
| 406 | 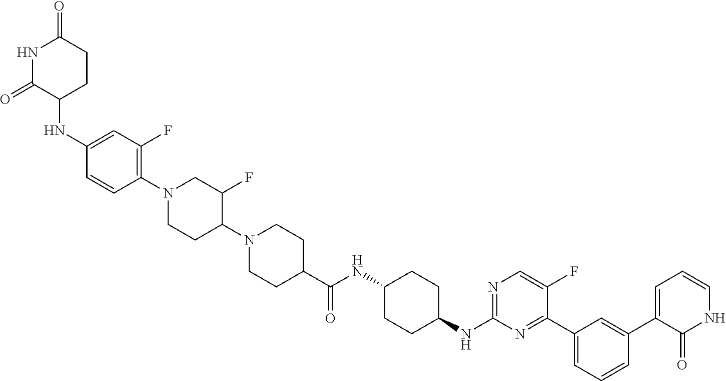 [SMILES: O=C1CCC(Nc2ccc(N3CCC(N4CCC(C(=O)N[C@H]5CC[C@H](Nc6ncc(F)c(-c7cccc(-c8ccc[nH]c8=O)c7)n6)CC5)CC4)C(F)C3)c(F)c2)C(=O)N1] |
| | 1′-(4-((2,6-dioxopiperidin-3-yl)amino)-2-fluorophenyl)-3′-fluoro-N-((1r,4r)-4-((5-fluoro-4-(3-(2-oxo-1,2-dihydro- |
| | pyridin-3-yl)phenyl)pyrimidin-2-yl)amino)cyclohexyl)-[1,4′-bipiperidine]-4-carboxamide |
| 407 | 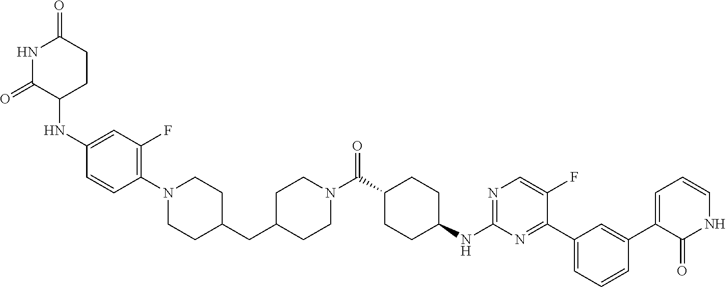 [SMILES: O=C1CCC(Nc2ccc(N3CCC(CC4CCN(C(=O)[C@H]5CC[C@H](Nc6ncc(F)c(-c7cccc(-c8ccc[nH]c8=O)c7)n6)CC5)CC4)CC3)c(F)c2)C(=O)N1] |
| | trans-3-((3-fluoro-4-(4-((1-(4-((5-fluoro-4-(3-(2-oxo-1,2-dihydropyridin-3-yl)phenyl)pyrimidin-2-yl)amino)cyclohexane- |
| | 1-carbonyl)piperidin-4-yl)methyl)piperidin-1-yl)phenyl)amino)piperidine-2,6-dione |
| 408 | 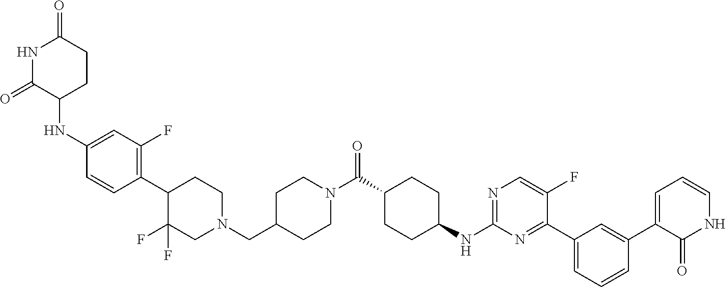 [SMILES: O=C1CCC(Nc2ccc(C3CCN(CC4CCN(C(=O)[C@H]5CC[C@H](Nc6ncc(F)c(-c7cccc(-c8ccc[nH]c8=O)c7)n6)CC5)CC4)CC3(F)F)c(F)c2)C(=O)N1] |
| | trans-3-[4-[3,3-difluoro-1-[1-[4-[5-fluoro-4-[3-(2-oxo-1H-pyridin-3-yl)phenyl]pyrimidin-2-yl]amino]cyclohexane- |
| | carbonyl]-4-piperidyl]methyl]-4-piperidyl]-3-fluoro-anilino]piperidine-2,6-dione |
| 409 | 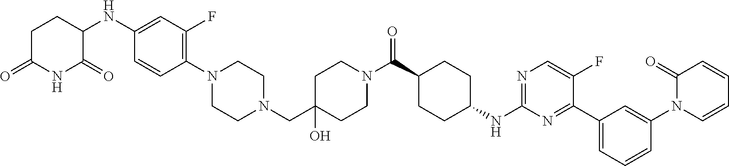 [SMILES: O=C1CCC(Nc2ccc(N3CCN(CC4(O)CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-3-((3-fluoro-4-(1-((1-(-4-((5-fluoro-4-(3-(2-oxopyridin-1(2H)-yl)phenyl)pyrimidin-2-yl)amino)cyclohexane-1- |
| | carbonyl)-4-hydroxypiperidin-4-yl)methyl)piperidin-4-yl)phenyl)amino)piperidine-2,6-dione |
| 410 | 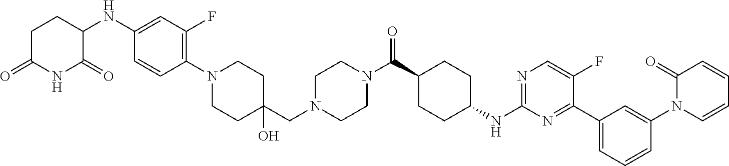 [SMILES: O=C1CCC(Nc2ccc(N3CCC(O)(CN4CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-3-((3-fluoro-4-(4-((4-(4-((5-fluoro-4-(3-(2-oxopyridin-1(2H)-yl)phenyl)pyrimidin-2-yl)amino)cyclohexane-1- |
| | carbonyl)piperazin-1-yl)methyl)-4-hydroxypiperidin-1-yl)phenyl)amino)piperidine-2,6-dione |
| 411 | 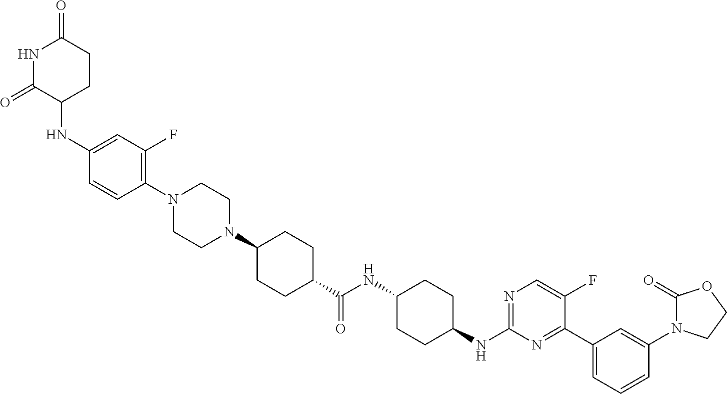 [SMILES: O=C1CCC(Nc2ccc(N3CCN([C@H]4CC[C@H](C(=O)N[C@H]5CC[C@H](Nc6ncc(F)c(-c7cccc(N8CCOC8=O)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperazin-1-yl)-N-((1r,4R)-4-((5-fluoro-4-(3- |
| | (2-oxooxazolidin-3-yl)phenyl)pyrimidin-2-yl)amino)cyclohexyl)cyclohexane-1-carboxamide |
| 412 | 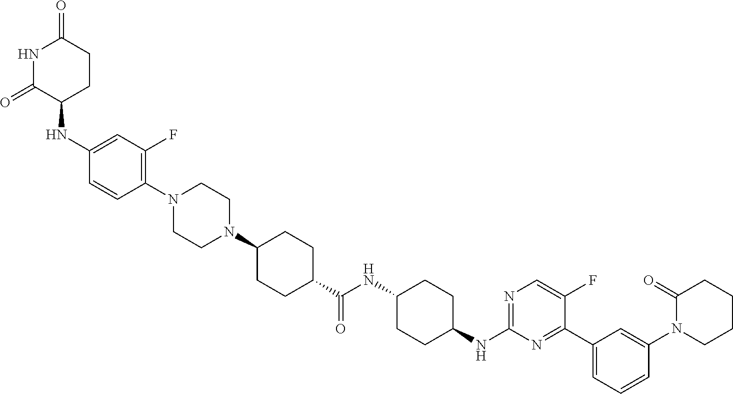 [SMILES: O=C1CC[C@@H](Nc2ccc(N3CCN([C@H]4CC[C@H](C(=O)N[C@H]5CC[C@H](Nc6ncc(F)c(-c7cccc(N8CCCCC8=O)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperazin-1-yl)-N-((1r,4R)-4-((5-fluoro-4-(3- |
| | (2-oxopiperidin-1-yl)phenyl)pyrimidin-2-yl)amino)cyclohexyl)cyclohexane-1-carboxamide |
| 413 | 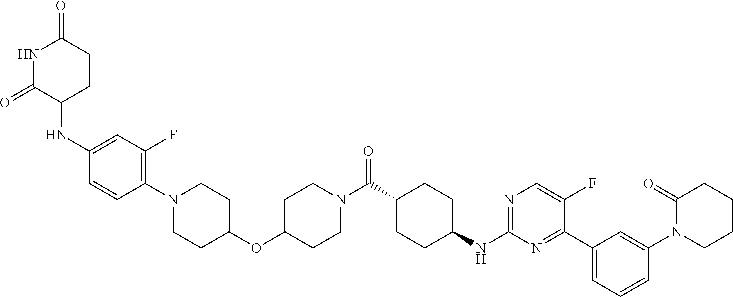 [SMILES: O=C1CCC(Nc2ccc(N3CCC(OC4CCN(C(=O)[C@H]5CC[C@H](Nc6ncc(F)c(-c7cccc(N8CCCCC8=O)c7)n6)CC5)CC4)CC3)c(F)c2)C(=O)N1] |
| | trans-3-((3-fluoro-4-(4-((1-(4-((5-fluoro-4-(3-(2-oxopiperidin-1-yl)phenyl)pyrimidin-2-yl)amino)cyclohexane-1- |
| | carbonyl)piperidin-4-yl)oxy)piperidin-1-yl)phenyl)amino)piperidine-2,6-dione |
| 414 | 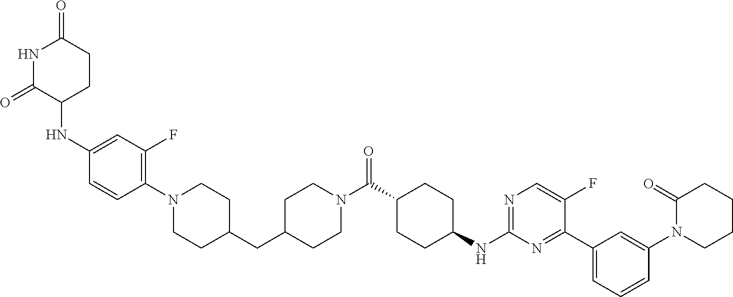 [SMILES: O=C1CCC(Nc2ccc(N3CCC(CC4CCN(C(=O)[C@H]5CC[C@H](Nc6ncc(F)c(-c7cccc(N8CCCCC8=O)c7)n6)CC5)CC4)CC3)c(F)c2)C(=O)N1] |
| | trans-3-((3-fluoro-4-(4-((1-(-4-((5-fluoro-4-(3-(2-oxopiperidin-1-yl)phenyl)pyrimidin-2-yl)amino)cyclohexane-1- |
| | carbonyl)piperidin-4-yl)methyl)piperidin-1-yl)phenyl)amino)piperidine-2,6-dione |
| 415 | 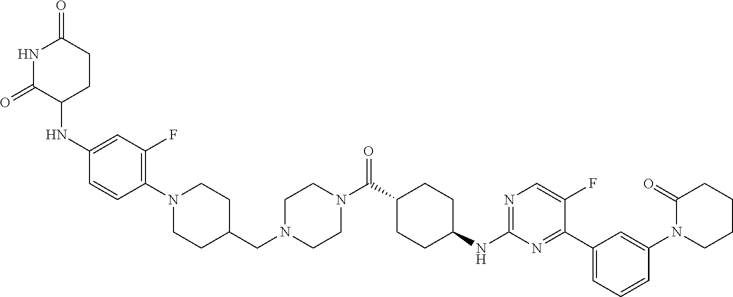 [SMILES: O=C1CCC(Nc2ccc(N3CCC(CN4CCN(C(=O)[C@H]5CC[C@H](Nc6ncc(F)c(-c7cccc(N8CCCCC8=O)c7)n6)CC5)CC4)CC3)c(F)c2)C(=O)N1] |
| | trans-3-((3-fluoro-4-(4-((4-(4-((5-fluoro-4-(3-(2-oxopiperidin-1-yl)phenyl)pyrimidin-2-yl)amino)cyclohexane-1- |
| | carbonyl)piperazin-1-yl)methyl)piperidin-1-yl)phenyl)amino)piperidine-2,6-dione |
| 416 | 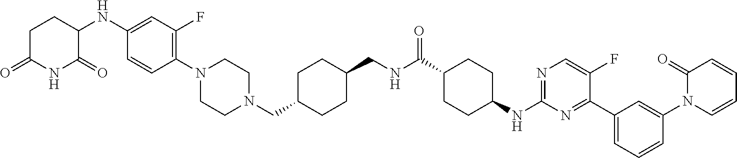 [SMILES: O=C1CCC(Nc2ccc(N3CCN(C[C@H]4CC[C@H](CNC(=O)[C@H]5CC[C@H](Nc6ncc(F)c(-c7cccc(-n8ccccc8=O)c7)n6)CC5)CC4)CC3)c(F)c2)C(=O)N1] |
| | trans-N-((trans-4-((4-(4-((2,6-dioxopiperidin-3-yl)amino)-2-fluorophenyl)piperazin-1-yl)methyl)cyclohexyl)methyl)- |
| | 4-((5-fluoro-4-(3-(2-oxopyridin-1(2H)-yl)phenyl)pyrimidin-2-yl)amino)cyclohexane-1-carboxamide |
| 417 | 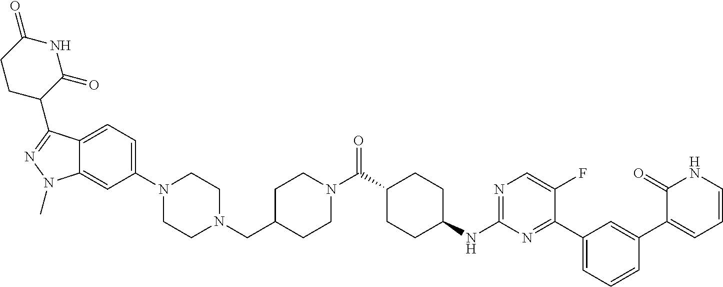 [SMILES: Cn1nc(C2CCC(=O)NC2=O)c2ccc(N3CCN(CC4CCN(C(=O)[C@H]5CC[C@H](Nc6ncc(F)c(-c7cccc(-c8ccc[nH]c8=O)c7)n6)CC5)CC4)CC3)cc21] |
| | 3-(6-(4-((1-(trans-4-((5-fluoro-4-(3-(2-oxo-1,2-dihydropyridin-3-yl)phenyl)pyrimidin-2-yl)amino)cyclohexane-1- |
| | carbonyl)piperidin-4-yl)methyl)piperazin-1-yl)-1-methyl-1H-indazol-3-yl)piperidine-2,6-dione |
| 418 | 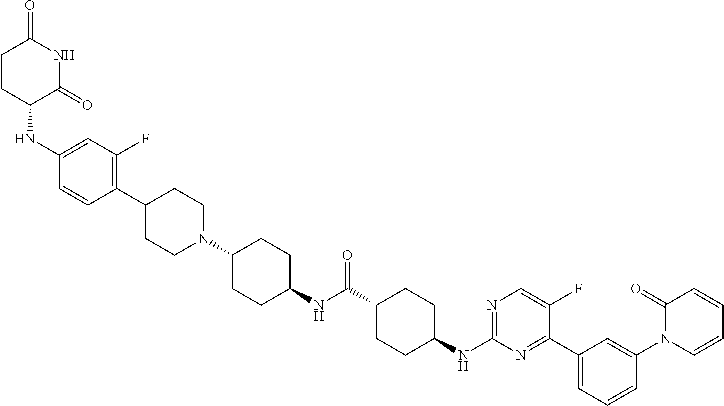 [SMILES: O=C1CC[C@@H](Nc2ccc(C3CCN([C@H]4CC[C@H](NC(=O)[C@H]5CC[C@H](Nc6ncc(F)c(-c7cccc(-n8ccccc8=O)c7)n6)CC5)CC4)CC3)c(F)c2)C(=O)N1] |
| | trans-(1r,4R)-N-((1R,4R)-4-(4-(4-(((R)-2,6-dioxopiperidin-3-yl)amino)-2-fluorophenyl)piperidin-1-yl)cyclohexyl)-4- |
| | ((5-fluoro-4-(3-(2-oxopyridin-1(2H)-yl)phenyl)pyrimidin-2-yl)amino)cyclohexane-1-carboxamide |
| 419 | 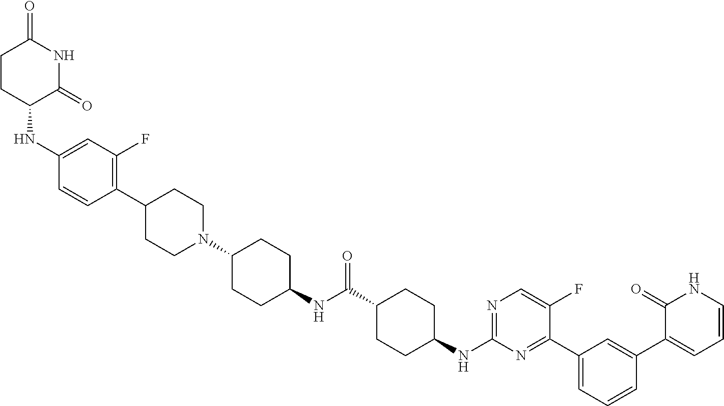 [SMILES: O=C1CC[C@@H](Nc2ccc(C3CCN([C@H]4CC[C@H](NC(=O)[C@H]5CC[C@H](Nc6ncc(F)c(-c7cccc(-c8ccc[nH]c8=O)c7)n6)CC5)CC4)CC3)c(F)c2)C(=O)N1] |
| | trans-(1r,4R)-N-((1R,4R)-4-(4-(4-(((R)-2,6-dioxopiperidin-3-yl)amino)-2-fluorophenyl)piperidin-1-yl)cyclohexyl)-4- |
| | ((5-fluoro-4-(3-(2-oxo-1,2-dihydropyridin-3-yl)phenyl)pyrimidin-2-yl)amino)cyclohexane-1-carboxamide |
| 420 | 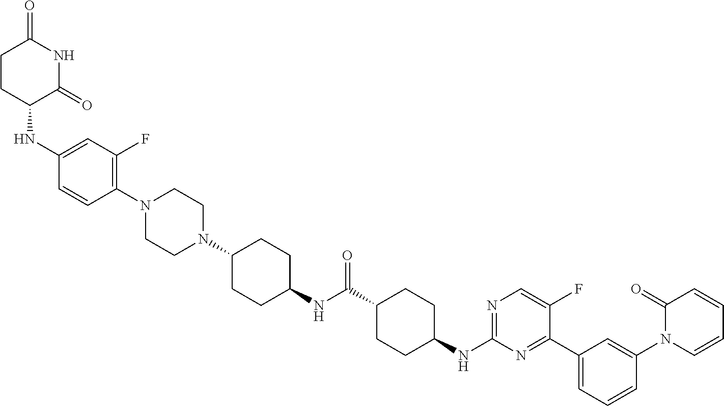 [SMILES: O=C1CC[C@@H](Nc2ccc(N3CCN([C@H]4CC[C@H](NC(=O)[C@H]5CC[C@H](Nc6ncc(F)c(-c7cccc(-n8ccccc8=O)c7)n6)CC5)CC4)CC3)c(F)c2)C(=O)N1] |
| | trans-(1r,4R)-N-((1R,4R)-4-(4-(4-(((R)-2,6-dioxopiperidin-3-yl)amino)-2-fluorophenyl)piperazin-1-yl)cyclohexyl)- |
| | 4-((5-fluoro-4-(3-(2-oxopyridin-1(2H)-yl)phenyl)pyrimidin-2-yl)amino)cyclohexane-1-carboxamide |
| 421 | 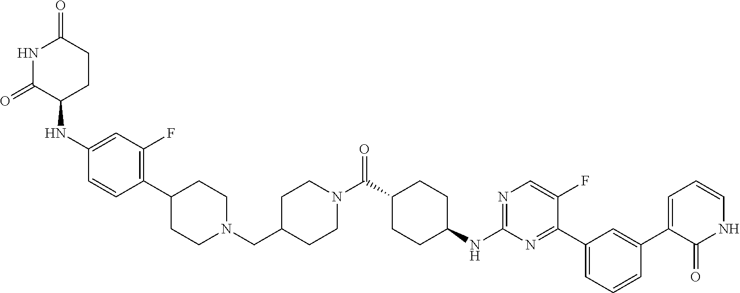 [SMILES: O=C1CC[C@@H](Nc2ccc(C3CCN(CC4CCN(C(=O)[C@H]5CC[C@H](Nc6ncc(F)c(-c7cccc(-c8ccc[nH]c8=O)c7)n6)CC5)CC4)CC3)c(F)c2)C(=O)N1] |
| | trans-(R)-3-((3-fluoro-4-(1-((1-(-4-((5-fluoro-4-(3-(2-oxo-1,2-dihydropyridin-3-yl)phenyl)pyrimidin-2-yl)amino)cyclo- |
| | hexane-1-carbonyl)piperidin-4-yl)methyl)piperidin-4-yl)phenyl)amino)piperidine-2,6-dione |
| 422 | 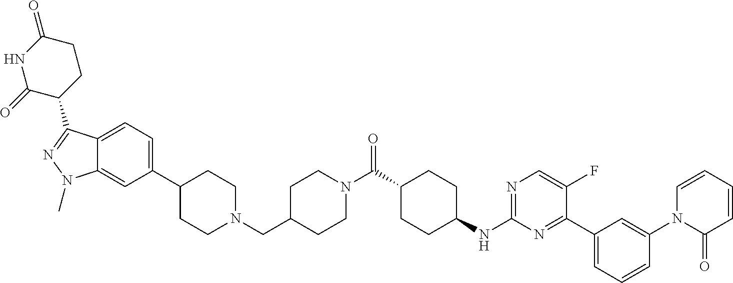 [SMILES: Cn1nc([C@H]2CCC(=O)NC2=O)c2ccc(C3CCN(CC4CCN(C(=O)[C@H]5CC[C@H](Nc6ncc(F)c(-c7cccc(-n8ccccc8=O)c7)n6)CC5)CC4)CC3)cc21] |
| | trans-(R)-3-(6-(1-((1-(4-((5-fluoro-4-(3-(2-oxopyridin-1(2H)-yl)phenyl)pyrimidin-2-yl)amino)cyclohexane-1- |
| | carbonyl)piperidin-4-yl)methyl)piperidin-4-yl)-1-methyl-1H-indazol-3-yl)piperidine-2,6-dione |
| 423 | 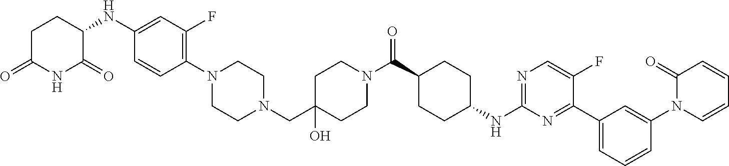 [SMILES: O=C1CC[C@H](Nc2ccc(N3CCN(CC4(O)CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-(R)-3-((3-fluoro-4-(4-((1-(4-((5-fluoro-4-(3-(2-oxopyridin-1(2H)-yl)phenyl)pyrimidin-2-yl)amino)cyclohexane- |
| | 1-carbonyl)-4-hydroxypiperidin-4-yl)methyl)piperazin-1-yl)phenyl)amino)piperidine-2,6-dione |
| 424 | 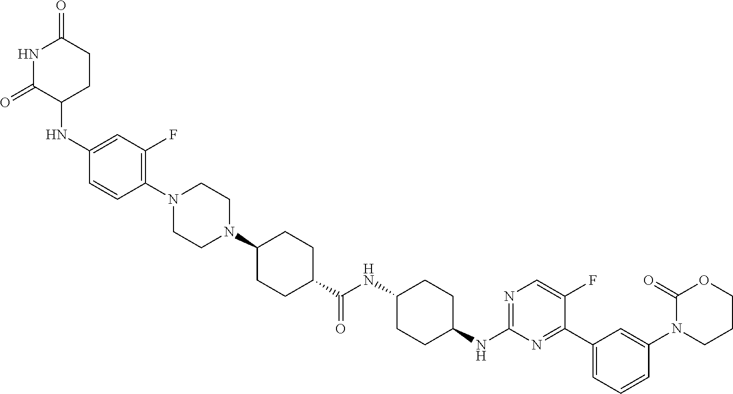 [SMILES: O=C1CCC(Nc2ccc(N3CCN([C@H]4CC[C@H](C(=O)N[C@H]5CC[C@H](Nc6ncc(F)c(-c7cccc(N8CCCOC8=O)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperazin-1-yl)-N-((1r,4R)-4-((5-fluoro-4-(3- |
| | (2-oxo-1,3-oxazinan-3-yl)phenyl)pyrimidin-2-yl)amino)cyclohexyl)cyclohexane-1-carboxamide |
| 425 | 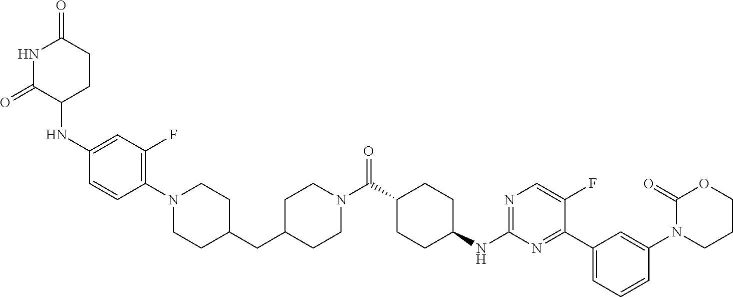 [SMILES: O=C1CCC(Nc2ccc(N3CCC(CC4CCN(C(=O)[C@H]5CC[C@H](Nc6ncc(F)c(-c7cccc(N8CCCOC8=O)c7)n6)CC5)CC4)CC3)c(F)c2)C(=O)N1] |
| | trans-3-((3-fluoro-4-(4-((1-(-4-((5-fluoro-4-(3-(2-oxo-1,3-oxazinan-3-yl)phenyl)pyrimidin-2-yl)amino)cyclohexane- |
| | 1-carbonyl)piperidin-4-yl)methyl)piperidin-1-yl)phenyl)amino)piperidine-2,6-dione |
| 426 | 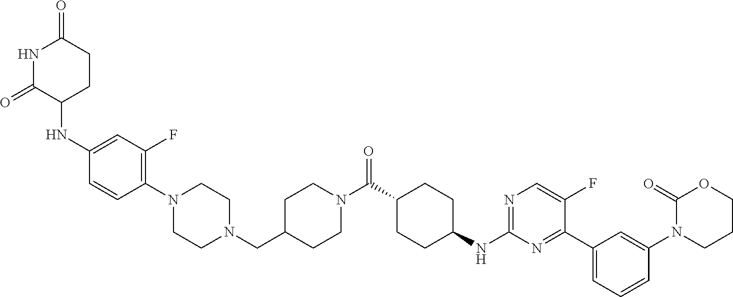 [SMILES: O=C1CCC(Nc2ccc(N3CCN(CC4CCN(C(=O)[C@H]5CC[C@H](Nc6ncc(F)c(-c7cccc(N8CCCOC8=O)c7)n6)CC5)CC4)CC3)c(F)c2)C(=O)N1] |
| | trans-3-[3-fluoro-4-[4-[[1-[4-[[5-fluoro-4-[3-(2-oxo-1,3-oxazinan-3-yl)phenyl]pyrimidin-2-yl]amino]cyclohexane- |
| | carbonyl]-4-piperidyl]methyl]piperazin-1-yl]anilino]piperidine-2,6-dione |
| 427 | 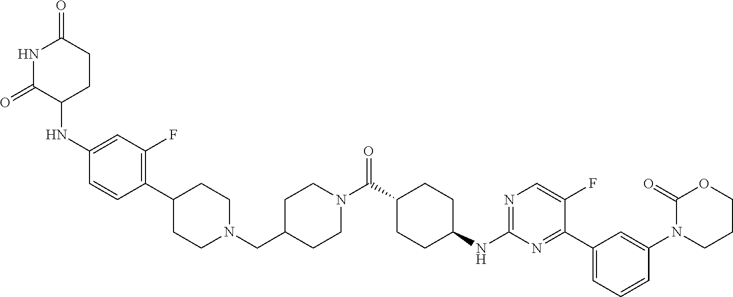 [SMILES: O=C1CCC(Nc2ccc(C3CCN(CC4CCN(C(=O)[C@H]5CC[C@H](Nc6ncc(F)c(-c7cccc(N8CCCOC8=O)c7)n6)CC5)CC4)CC3)c(F)c2)C(=O)N1] |
| | trans-3-((3-fluoro-4-(1-((1-(4-((5-fluoro-4-(3-(2-oxo-1,3-oxazinan-3-yl)phenyl)pyrimidin-2-yl)amino)cyclohexane- |
| | 1-carbonyl)piperidin-4-yl)methyl)piperidin-4-yl)phenyl)amino)piperidine-2,6-dione |
| 428 | 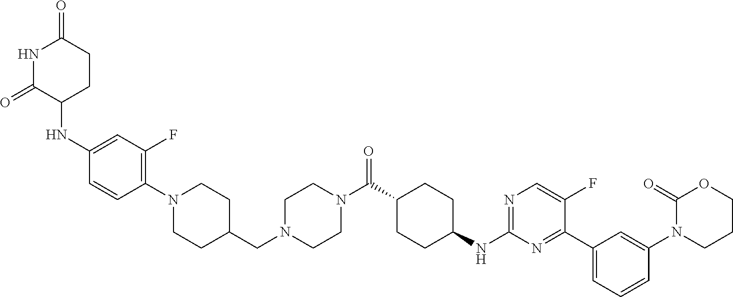 [SMILES: O=C1CCC(Nc2ccc(N3CCC(CN4CCN(C(=O)[C@H]5CC[C@H](Nc6ncc(F)c(-c7cccc(N8CCCOC8=O)c7)n6)CC5)CC4)CC3)c(F)c2)C(=O)N1] |
| | trans-3-((3-fluoro-4-(4-((4-(4-((5-fluoro-4-(3-(2-oxo-1,3-oxazinan-3-yl)phenyl)pyrimidin-2-yl)amino)cyclohexane- |
| | 1-carbonyl)piperazin-1-yl)methyl)piperidin-1-yl)phenyl)amino)piperidine-2,6-dione |
| 429 | 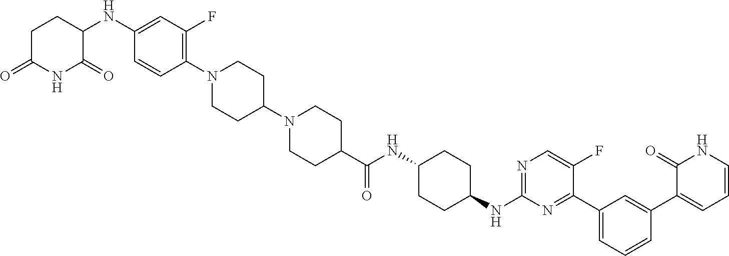 [SMILES: O=C1CCC(Nc2ccc(N3CCC(N4CCC(C(=O)N[C@H]5CC[C@H](Nc6ncc(F)c(-c7cccc(-c8ccc[nH]c8=O)c7)n6)CC5)CC4)CC3)c(F)c2)C(=O)N1] |
| | trans-1′-(4-((2,6-dioxopiperidin-3-yl)amino)-2-fluorophenyl)-N-(4-((5-fluoro-4-(3-(2-oxo-1,2-dihydropyridin-3- |
| | yl)phenyl)pyrimidin-2-yl)amino)cyclohexyl)-[1,4′-bipiperidine]-4-carboxamide |
| 430 | 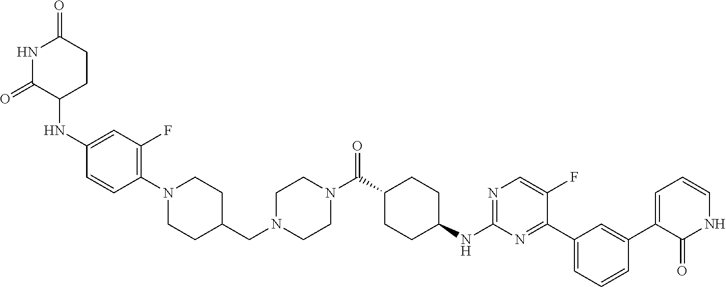 [SMILES: O=C1CCC(Nc2ccc(N3CCC(CN4CCN(C(=O)[C@H]5CC[C@H](Nc6ncc(F)c(-c7cccc(-c8ccc[nH]c8=O)c7)n6)CC5)CC4)CC3)c(F)c2)C(=O)N1] |
| | 3-((3-fluoro-4-(4-((4-(trans-4-((5-fluoro-4-(3-(2-oxo-1,2-dihydropyridin-3-yl)phenyl)pyrimidin-2-yl)amino)cyclo- |
| | hexane-1-carbonyl)piperazin-1-yl)methyl)piperidin-1-yl)phenyl)amino)piperidine-2,6-dione |
| 431 | 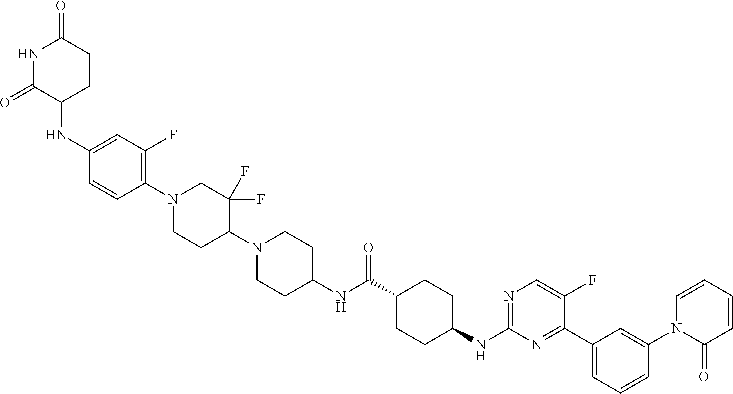 [SMILES: O=C1CCC(Nc2ccc(N3CCC(N4CCC(NC(=O)[C@H]5CC[C@H](Nc6ncc(F)c(-c7cccc(-n8ccccc8=O)c7)n6)CC5)CC4)C(F)(F)C3)c(F)c2)C(=O)N1] |
| | trans-N-(1′-(4-((2,6-dioxopiperidin-3-yl)amino)-2-fluorophenyl)-3′,3′-difluoro-[1,4′-bipiperidin]-4-yl)-4-((5-fluoro-4- |
| | (3-(2-oxopyridin-1(2H)-yl)phenyl)pyrimidin-2-yl)amino)cyclohexane-1-carboxamide |
| 432 | 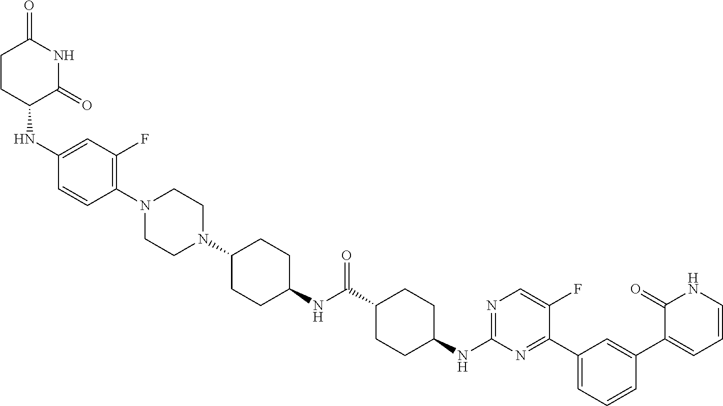 [SMILES: O=C1CC[C@@H](Nc2ccc(N3CCN([C@H]4CC[C@H](NC(=O)[C@H]5CC[C@H](Nc6ncc(F)c(-c7cccc(-c8ccc[nH]c8=O)c7)n6)CC5)CC4)CC3)c(F)c2)C(=O)N1] |
| | trans-(1r,4R)-N-((1R,4R)-4-(4-(4-(((R)-2,6-dioxopiperidin-3-yl)amino)-2-fluorophenyl)piperazin-1-yl)cyclohexyl)-4- |
| | ((5-fluoro-4-(3-(2-oxo-1,2-dihydropyridin-3-yl)phenyl)pyrimidin-2-yl)amino)cyclohexane-1-carboxamide |
| 433 | 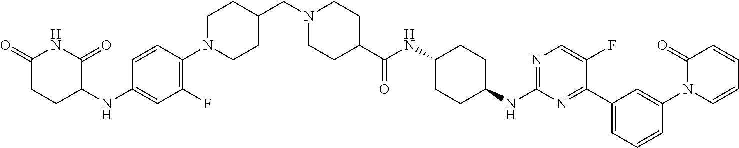 [SMILES: O=C1CCC(Nc2ccc(N3CCC(CN4CCC(C(=O)N[C@H]5CC[C@H](Nc6ncc(F)c(-c7cccc(-n8ccccc8=O)c7)n6)CC5)CC4)CC3)c(F)c2)C(=O)N1] |
| | 1-((1-(4-((2,6-dioxopiperidin-3-yl)amino)-2-fluorophenyl)piperidin-4-yl)methyl)-N-((1r,4r)-4-((5-fluoro-4-(3-(2- |
| | oxopyridin-1(2H)-yl)phenyl)pyrimidin-2-yl)amino)cyclohexyl)piperidine-4-carboxamide |
| 434 | 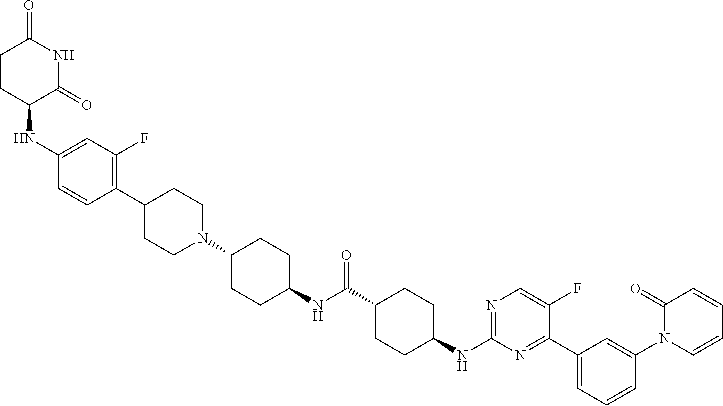 [SMILES: O=C1CC[C@H](Nc2ccc(C3CCN([C@H]4CC[C@H](NC(=O)[C@H]5CC[C@H](Nc6ncc(F)c(-c7cccc(-n8ccccc8=O)c7)n6)CC5)CC4)CC3)c(F)c2)C(=O)N1] |
| | trans-(1r,4R)-N-((1S,4R)-4-(4-(4-(((S)-2,6-dioxopiperidin-3-yl)amino)-2-fluorophenyl)piperidin-1-yl)cyclohexyl)-4- |
| | ((5-fluoro-4-(3-(2-oxopyridin-1(2H)-yl)phenyl)pyrimidin-2-yl)amino)cyclohexane-1-carboxamide |
| 435 | 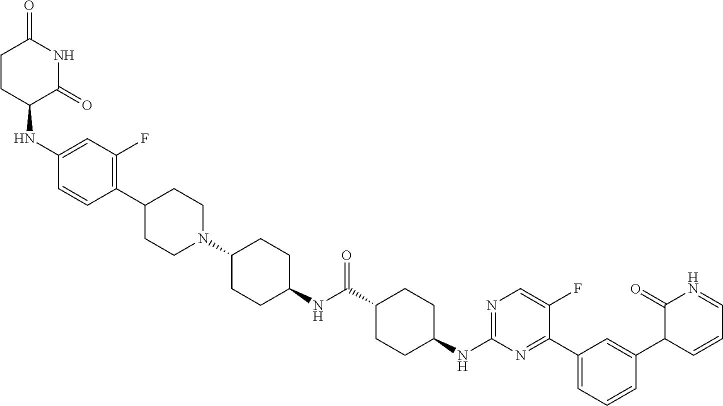 |
| | trans-(1r,4R)-N-((1S,4R)-4-(4-(4-(((S)-2,6-dioxopiperidin-3-yl)amino)-2-fluorophenyl)piperidin-1-yl)cyclohexyl)-4-((5- |
| | fluoro-4-(3-(2-oxo-1,2-dihydropyridin-3-yl)phenyl)pyrimidin-2-yl)amino)cyclohexane-1-carboxamide |
| 436 | 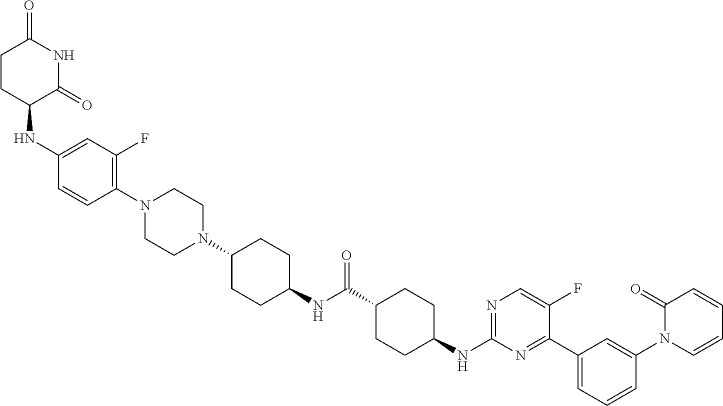 [SMILES: O=C1CC[C@H](Nc2ccc(N3CCN([C@H]4CC[C@H](NC(=O)[C@H]5CC[C@H](Nc6ncc(F)c(-c7cccc(-n8ccccc8=O)c7)n6)CC5)CC4)CC3)c(F)c2)C(=O)N1] |
| | trans-(1r,4R)-N-((1S,4R)-4-(4-(4-(((S)-2,6-dioxopiperidin-3-yl)amino)-2-fluorophenyl)piperazin-1-yl)cyclohexyl)-4- |
| | ((5-fluoro-4-(3-(2-oxopyridin-1(2H)-yl)phenyl)pyrimidin-2-yl)amino)cyclohexane-1-carboxamide |
| 437 | 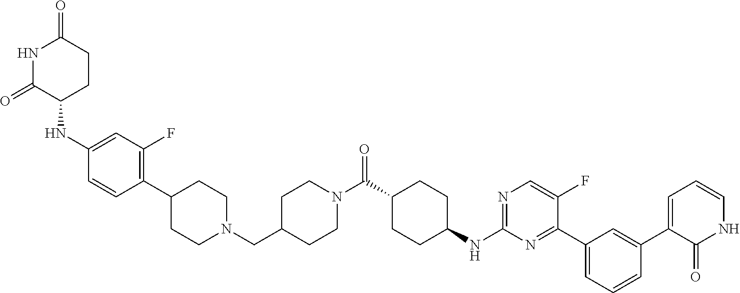 [SMILES: O=C1CC[C@H](Nc2ccc(C3CCN(CC4CCN(C(=O)[C@H]5CC[C@H](Nc6ncc(F)c(-c7cccc(-c8ccc[nH]c8=O)c7)n6)CC5)CC4)CC3)c(F)c2)C(=O)N1] |
| | trans-(S)-3-((3-fluoro-4-(1-((1-(-4-((5-fluoro-4-(3-(2-oxo-1,2-dihydropyridin-3-yl)phenyl)pyrimidin-2-yl)amino)cyclo- |
| | hexane-1-carbonyl)piperidin-4-yl)methyl)piperidin-4-yl)phenyl)amino)piperidine-2,6-dione |
| 438 | 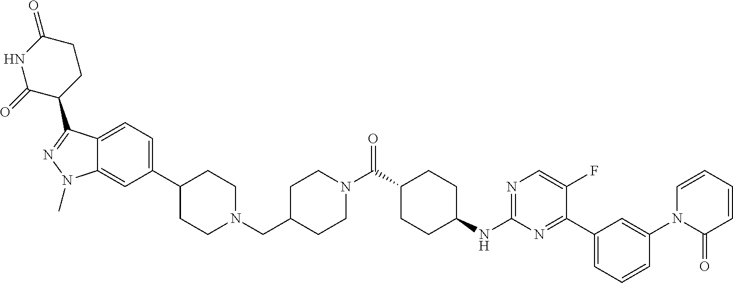 [SMILES: Cn1nc([C@@H]2CCC(=O)NC2=O)c2ccc(C3CCN(CC4CCN(C(=O)[C@H]5CC[C@H](Nc6ncc(F)c(-c7cccc(-n8ccccc8=O)c7)n6)CC5)CC4)CC3)cc21] |
| | trans-(S)-3-(6-(1-((1-(4-((5-fluoro-4-(3-(2-oxopyridin-1(2H)-yl)phenyl)pyrimidin-2-yl)amino)cyclohexane-1- |
| | carbonyl)piperidin-4-yl)methyl)piperidin-4-yl)-1-methyl-1H-indazol-3-yl)piperidine-2,6-dione |
| 439 | 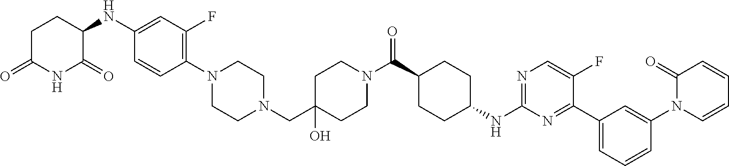 [SMILES: O=C1CC[C@@H](Nc2ccc(N3CCN(CC4(O)CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-(S)-3-((3-fluoro-4-(4-((1-(4-((5-fluoro-4-(3-(2-oxopyridin-1(2H)-yl)phenyl)pyrimidin-2-yl)amino)cyclohexane- |
| | 1-carbonyl)-4-hydroxypiperidin-4-yl)methyl)piperazin-1-yl)phenyl)amino)piperidine-2,6-dione |
| 440 | 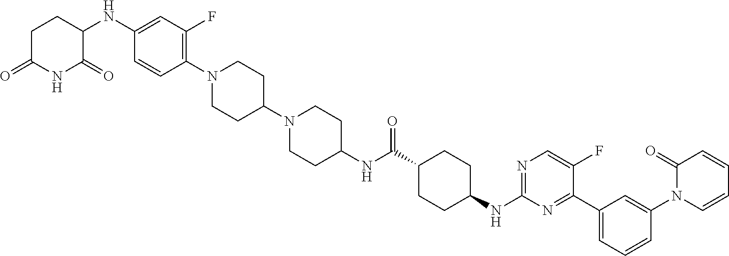 [SMILES: O=C1CCC(Nc2ccc(N3CCC(N4CCC(NC(=O)[C@H]5CC[C@H](Nc6ncc(F)c(-c7cccc(-n8ccccc8=O)c7)n6)CC5)CC4)CC3)c(F)c2)C(=O)N1] |
| | trans-N-(1′-(4-(((R)-2,6-dioxopiperidin-3-yl)amino)-2-fluorophenyl)-[1,4′-bipiperidin]-4-yl)-4-((5-fluoro-4-(3-(2- |
| | oxopyridin-1(2H)-yl)phenyl)pyrimidin-2-yl)amino)cyclohexane-1-carboxamide |
| 441 | 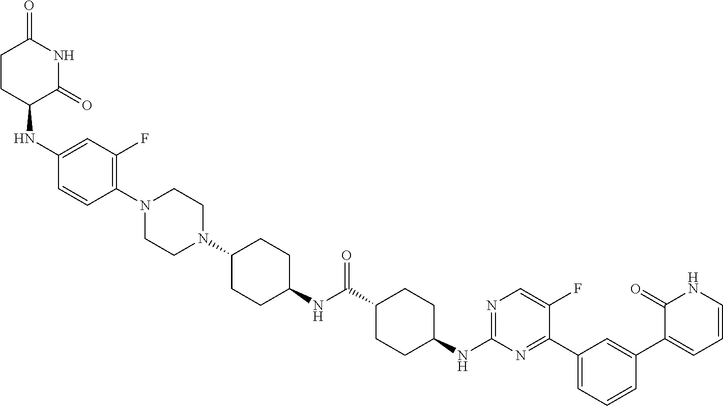 [SMILES: O=C1CC[C@H](Nc2ccc(N3CCN([C@H]4CC[C@H](NC(=O)[C@H]5CC[C@H](Nc6ncc(F)c(-c7cccc(-c8ccc[nH]c8=O)c7)n6)CC5)CC4)CC3)c(F)c2)C(=O)N1] |
| | trans-(1r,4R)-N-((1S,4R)-4-(4-(4-(((S)-2,6-dioxopiperidin-3-yl)amino)-2-fluorophenyl)piperazin-1-yl)cyclohexyl)-4- |
| | ((5-fluoro-4-(3-(2-oxo-1,2-dihydropyridin-3-yl)phenyl)pyrimidin-2-yl)amino)cyclohexane-1-carboxamide |
| 442 | 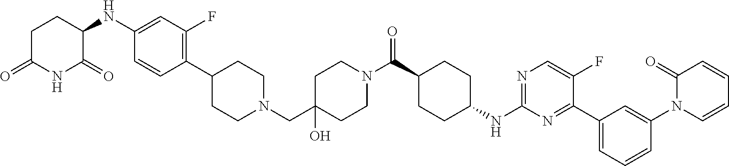 [SMILES: O=C1CC[C@@H](Nc2ccc(C3CCN(CC4(O)CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-(R)-3-((3-fluoro-4-(1-((1-(-4-((5-fluoro-4-(3-(2-oxopyridin-1(2H)-yl)phenyl)pyrimidin-2-yl)amino)cyclohexane- |
| | 1-carbonyl)-4-hydroxypiperidin-4-yl)methyl)piperidin-4-yl)phenyl)amino)piperidine-2,6-dione |
| 443 | 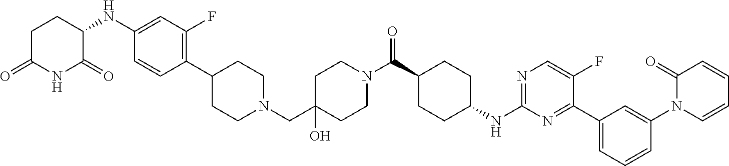 [SMILES: O=C1CC[C@H](Nc2ccc(C3CCN(CC4(O)CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-(S)-3-((3-fluoro-4-(1-((1-(-4-((5-fluoro-4-(3-(2-oxopyridin-1(2H)-yl)phenyl)pyrimidin-2-yl)amino)cyclohexane- |
| | 1-carbonyl)-4-hydroxypiperidin-4-yl)methyl)piperidin-4-yl)phenyl)amino)piperidine-2,6-dione |
| 444 | 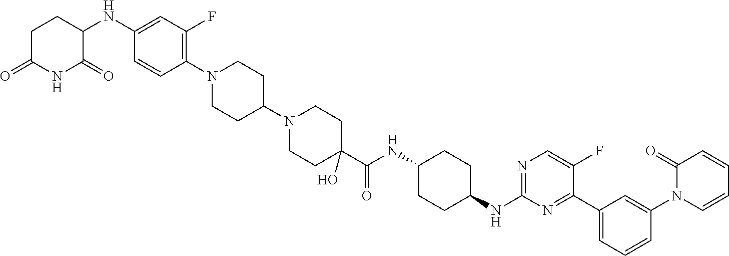 [SMILES: O=C1CCC(Nc2ccc(N3CCC(N4CCC(O)(C(=O)N[C@H]5CC[C@H](Nc6ncc(F)c(-c7cccc(-n8ccccc8=O)c7)n6)CC5)CC4)CC3)c(F)c2)C(=O)N1] |
| | trans-1′-(4-((2,6-dioxopiperidin-3-yl)amino)-2-fluorophenyl)-N-(4-((5-fluoro-4-(3-(2-oxopyridin-1(2H)-yl)phenyl)- |
| | pyrimidin-2-yl)amino)cyclohexyl)-4-hydroxy-[1,4′-bipiperidine]-4-carboxamide |
| 445 | 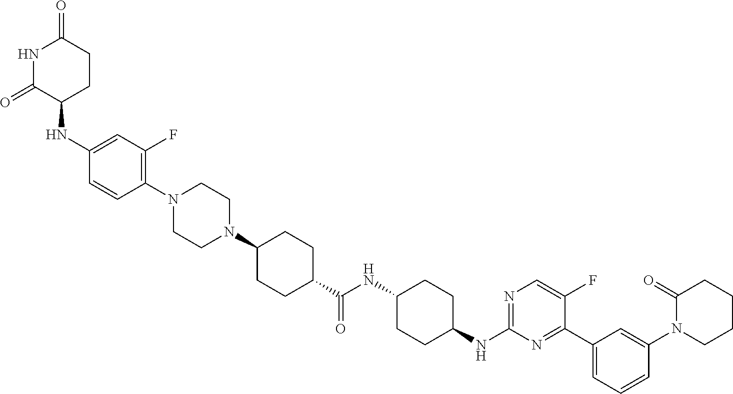 [SMILES: O=C1CC[C@@H](Nc2ccc(N3CCN([C@H]4CC[C@H](C(=O)N[C@H]5CC[C@H](Nc6ncc(F)c(-c7cccc(N8CCCCC8=O)c7)n6)CC5)CC4)CC3)c(F)c2)C(=O)N1] |
| | trans-(1R,4R)-4-(4-(4-(((R)-2,6-dioxopiperidin-3-yl)amino)-2-fluorophenyl)piperazin-1-yl)-N-((1r,4R)-4-((5-fluoro- |
| | 4-(3-(2-oxopiperidin-1-yl)phenyl)pyrimidin-2-yl)amino)cyclohexyl)cyclohexane-1-carboxamide |
| 446 | 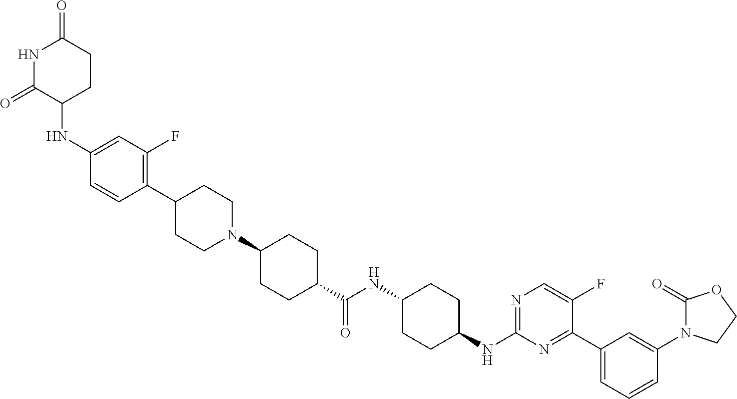 [SMILES: O=C1CCC(Nc2ccc(C3CCN([C@H]4CC[C@H](C(=O)N[C@H]5CC[C@H](Nc6ncc(F)c(-c7cccc(N8CCOC8=O)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4-(3-(2- |
| | oxooxazolidin-3-yl)phenyl)pyrimidin-2-yl)amino)cyclohexyl)cyclohexane-1-carboxamide |
| 447 | 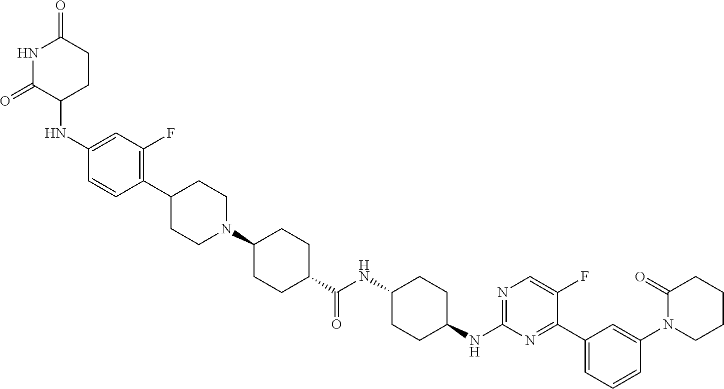 [SMILES: O=C1CCC(Nc2ccc(C3CCN([C@H]4CC[C@H](C(=O)N[C@H]5CC[C@H](Nc6ncc(F)c(-c7cccc(N8CCCCC8=O)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4-(3- |
| | (2-oxopiperidin-1-yl)phenyl)pyrimidin-2-yl)amino)cyclohexyl)cyclohexane-1-carboxamide |
| 448 | 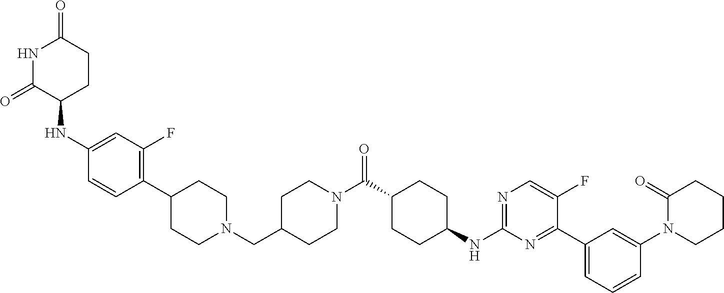 [SMILES: O=C1CC[C@@H](Nc2ccc(C3CCN(CC4CCN(C(=O)[C@H]5CC[C@H](Nc6ncc(F)c(-c7cccc(N8CCCCC8=O)c7)n6)CC5)CC4)CC3)c(F)c2)C(=O)N1] |
| | trans-(R)-3-((3-fluoro-4-(1-((1-((1r,4r)-4-((5-fluoro-4-(3-(2-oxopiperidin-1-yl)phenyl)pyrimidin-2-yl)amino)cyclo- |
| | hexane-1-carbonyl)piperidin-4-yl)methyl)piperidin-4-yl)phenyl)amino)piperidine-2,6-dione |
| 449 | 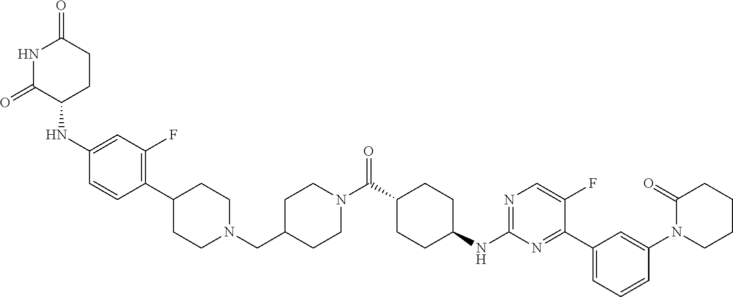 [SMILES: O=C1CC[C@H](Nc2ccc(C3CCN(CC4CCN(C(=O)[C@H]5CC[C@H](Nc6ncc(F)c(-c7cccc(N8CCCCC8=O)c7)n6)CC5)CC4)CC3)c(F)c2)C(=O)N1] |
| | trans-(S)-3-((3-fluoro-4-(1-((1-((1r,4r)-4-((5-fluoro-4-(3-(2-oxopiperidin-1-yl)phenyl)pyrimidin-2-yl)amino)cyclohexane- |
| | 1-carbonyl)piperidin-4-yl)methyl)piperidin-4-yl)phenyl)amino)piperidine-2,6-dione |
| 450 | 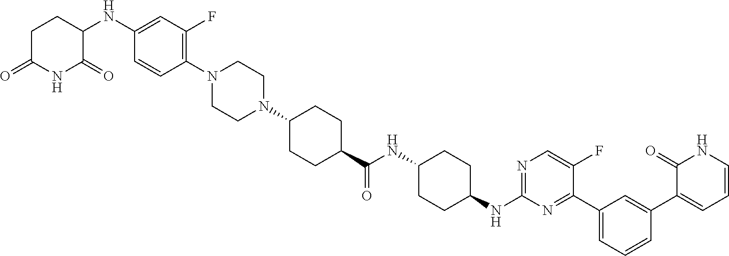 [SMILES: O=C1CCC(Nc2ccc(N3CCN([C@H]4CC[C@H](C(=O)N[C@H]5CC[C@H](Nc6ncc(F)c(-c7cccc(-c8ccc[nH]c8=O)c7)n6)CC5)CC4)CC3)c(F)c2)C(=O)N1] |
| | trans-4-(4-(4-((2,6-dioxopiperidin-3-yl)amino)-2-fluorophenyl)piperazin-1-yl)-N-(trans-4-((5-fluoro-4-(3-(2-oxo-1,2- |
| | dihydropyridin-3-yl)phenyl)pyrimidin-2-yl)amino)cyclohexyl)cyclohexane-1-carboxamide |
| 451 | 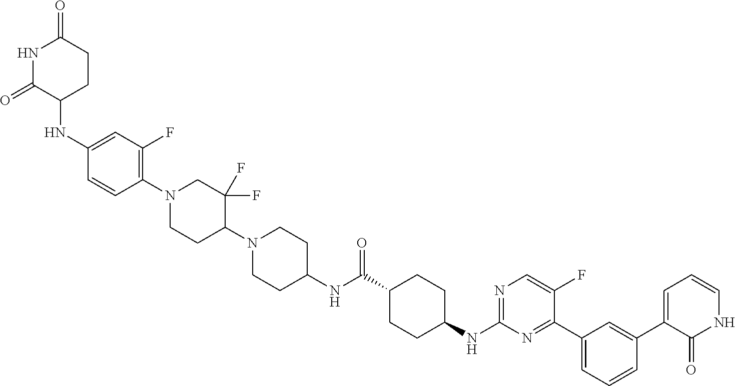 [SMILES: O=C1CCC(Nc2ccc(N3CCC(N4CCC(NC(=O)[C@H]5CC[C@H](Nc6ncc(F)c(-c7cccc(-c8ccc[nH]c8=O)c7)n6)CC5)CC4)C(F)(F)C3)c(F)c2)C(=O)N1] |
| | trans-N-(1′-(4-((2,6-dioxopiperidin-3-yl)amino)-2-fluorophenyl)-3′,3′-difluoro-[1,4′-bipiperidin]-4-yl)-4-((5-fluoro-4-(3- |
| | (2-oxo-1,2-dihydropyridin-3-yl)phenyl)pyrimidin-2-yl)amino)cyclohexane-1-carboxamide |
| 452 | 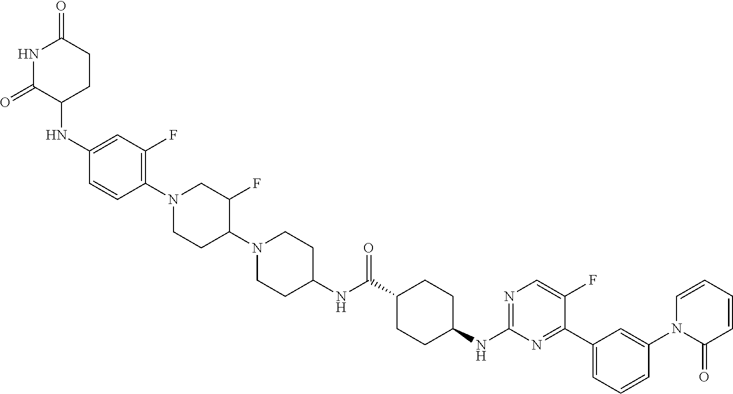 [SMILES: O=C1CCC(Nc2ccc(N3CCC(N4CCC(NC(=O)[C@H]5CC[C@H](Nc6ncc(F)c(-c7cccc(-n8ccccc8=O)c7)n6)CC5)CC4)C(F)C3)c(F)c2)C(=O)N1] |
| | trans-N-(1′-(4-((2,6-dioxopiperidin-3-yl)amino)-2-fluorophenyl)-3′-fluoro-[1,4′-bipiperidin]-4-yl)-4-((5-fluoro-4-(3- |
| | (2-oxopyridin-1(2H)-yl)phenyl)pyrimidin-2-yl)amino)cyclohexane-1-carboxamide |
| 453 | 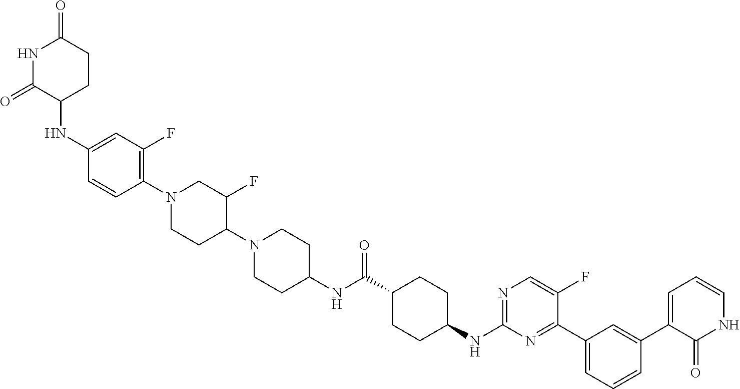 [SMILES: O=C1CCC(Nc2ccc(N3CCC(N4CCC(NC(=O)[C@H]5CC[C@H](Nc6ncc(F)c(-c7cccc(-c8ccc[nH]c8=O)c7)n6)CC5)CC4)C(F)C3)c(F)c2)C(=O)N1] |
| | trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(2- |
| | oxo-1,2-dihydropyridin-3-yl)phenyl)pyrimidin-2-yl)amino)cyclohexane-1-carboxamide |
| 454 | 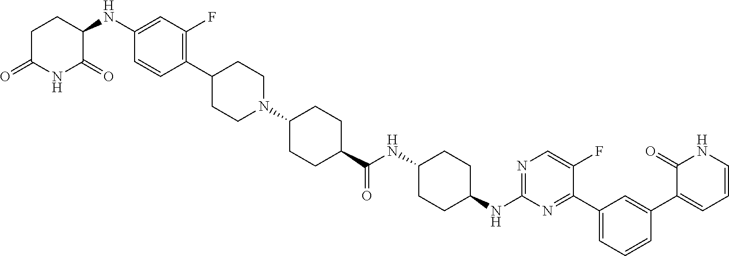 [SMILES: O=C1CC[C@@H](Nc2ccc(C3CCN([C@H]4CC[C@H](C(=O)N[C@H]5CC[C@H](Nc6ncc(F)c(-c7cccc(-c8ccc[nH]c8=O)c7)n6)CC5)CC4)CC3)c(F)c2)C(=O)N1] |
| | trans-4-(4-(4-(((R)-2,6-dioxopiperidin-3-yl)amino)-2-fluorophenyl)piperidin-1-yl)-N-(trans-4-((5-fluoro-4-(3-(2-oxo- |
| | 1,2-dihydropyridin-3-yl)phenyl)pyrimidin-2-yl)amino)cyclohexyl)cyclohexane-1-carboxamide |
| 455 | 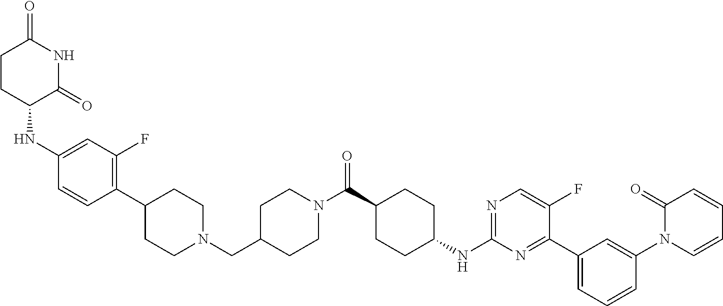 [SMILES: O=C1CC[C@@H](Nc2ccc(C3CCN(CC4CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-(R)-3-((3-fluoro-4-(1-((1-((1r,4r)-4-((5-fluoro-4-(3-(2-oxopyridin-1(2H)-yl)phenyl)pyrimidin-2-yl)amino)cyclo- |
| | hexane-1-carbonyl)piperidin-4-yl)methyl)piperidin-4-yl)phenyl)amino)piperidine-2,6-dione |
| 456 | 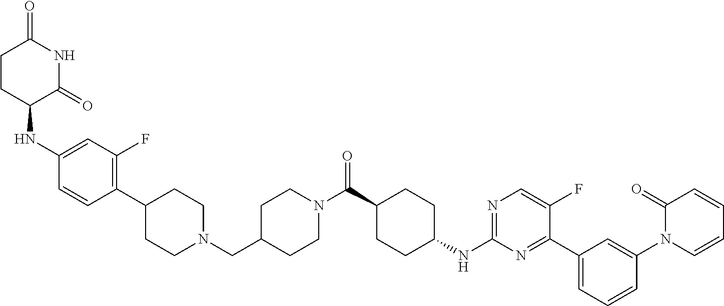 [SMILES: O=C1CC[C@H](Nc2ccc(C3CCN(CC4CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-(S)-3-((3-fluoro-4-(1-((1-((1r,4r)-4-((5-fluoro-4-(3-(2-oxopyridin-1(2H)-yl)phenyl)pyrimidin-2-yl)amino)cyclo- |
| | hexane-1-carbonyl)piperidin-4-yl)methyl)piperidin-4-yl)phenyl)amino)piperidine-2,6-dione |
| 457 | 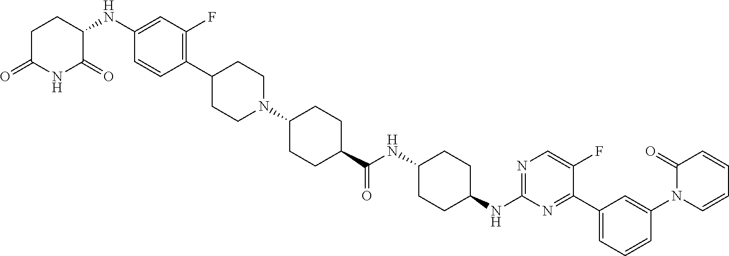 [SMILES: O=C1CC[C@H](Nc2ccc(C3CCN([C@H]4CC[C@H](C(=O)N[C@H]5CC[C@H](Nc6ncc(F)c(-c7cccc(-n8ccccc8=O)c7)n6)CC5)CC4)CC3)c(F)c2)C(=O)N1] |
| | (1S,4R)-4-(4-(4-(((S)-2,6-dioxopiperidin-3-yl)amino)-2-fluorophenyl)piperidin-1-yl)-N-((1r,4R)-4-((5-fluoro-4-(3-(2- |
| | oxopyridin-1(2H)-yl)phenyl)pyrimidin-2-yl)amino)cyclohexyl)cyclohexane-1-carboxamide |
| 458 | 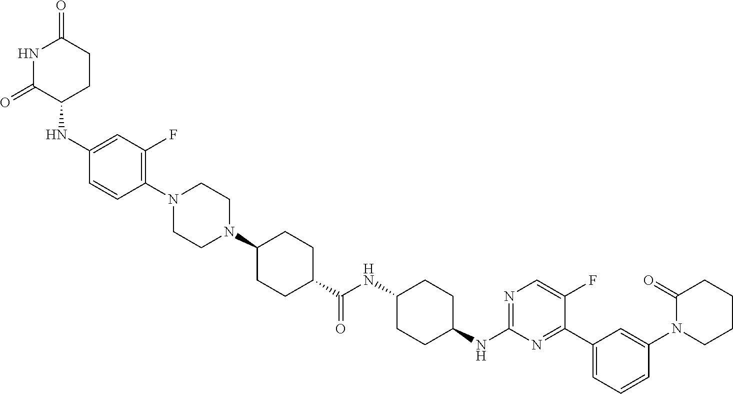 [SMILES: O=C1CC[C@H](Nc2ccc(N3CCN([C@H]4CC[C@H](C(=O)N[C@H]5CC[C@H](Nc6ncc(F)c(-c7cccc(N8CCCCC8=O)c7)n6)CC5)CC4)CC3)c(F)c2)C(=O)N1] |
| | trans-(1S,4R)-4-(4-(4-(((S)-2,6-dioxopiperidin-3-yl)amino)-2-fluorophenyl)piperazin-1-yl)-N-((1r,4R)-4-((5-fluoro- |
| | 4-(3-(2-oxopiperidin-1-yl)phenyl)pyrimidin-2-yl)amino)cyclohexyl)cyclohexane-1-carboxamide |
| 459 | 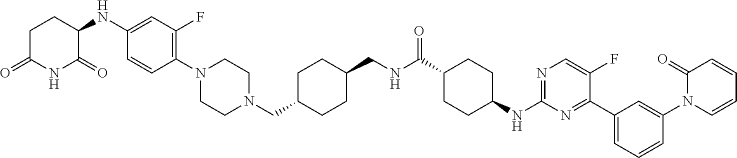 [SMILES: O=C1CC[C@@H](Nc2ccc(N3CCN(C[C@H]4CC[C@H](CNC(=O)[C@H]5CC[C@H](Nc6ncc(F)c(-c7cccc(-n8ccccc8=O)c7)n6)CC5)CC4)CC3)c(F)c2)C(=O)N1] |
| | (1r,4R)-N-(((1R,4R)-4-((4-(4-(((R)-2,6-dioxopiperidin-3-yl)amino)-2-fluorophenyl)piperazin-1-yl)methyl)cyclohexyl)- |
| | methyl)-4-((5-fluoro-4-(3-(2-oxopyridin-1(2H)-yl)phenyl)pyrimidin-2-yl)amino)cyclohexane-1-carboxamide |
| 460 | 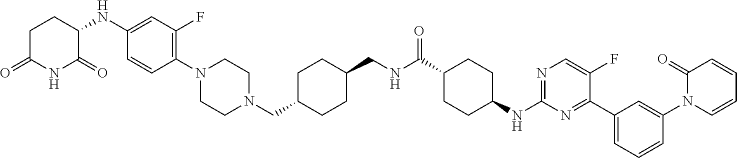 [SMILES: O=C1CC[C@H](Nc2ccc(N3CCN(C[C@H]4CC[C@H](CNC(=O)[C@H]5CC[C@H](Nc6ncc(F)c(-c7cccc(-n8ccccc8=O)c7)n6)CC5)CC4)CC3)c(F)c2)C(=O)N1] |
| | (1r,4R)-N-(((1S,4R)-4-((4-(4-(((S)-2,6-dioxopiperidin-3-yl)amino)-2-fluorophenyl)piperazin-1-yl)methyl)cyclohexyl)- |
| | methyl)-4-((5-fluoro-4-(3-(2-oxopyridin-1(2H)-yl)phenyl)pyrimidin-2-yl)amino)cyclohexane-1-carboxamide |
| 461 | 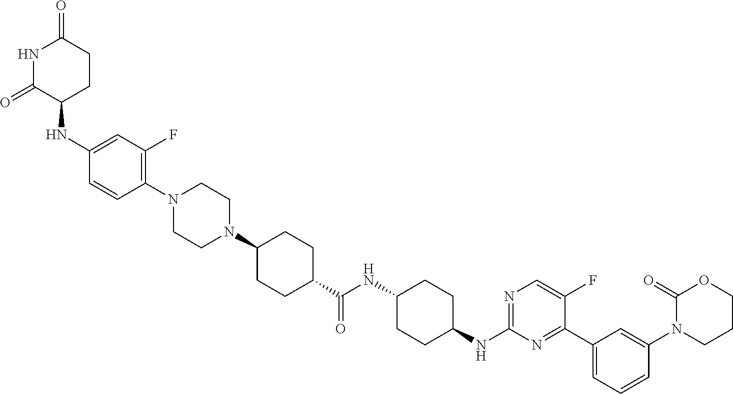 [SMILES: O=C1CC[C@@H](Nc2ccc(N3CCN([C@H]4CC[C@H](C(=O)N[C@H]5CC[C@H](Nc6ncc(F)c(-c7cccc(N8CCCOC8=O)c7)n6)CC5)CC4)CC3)c(F)c2)C(=O)N1] |
| | trans-(1R,4R)-4-(4-(4-(((R)-2,6-dioxopiperidin-3-yl)amino)-2-fluorophenyl)piperazin-1-yl)-N-((1r,4R)-4-((5-fluoro- |
| | 4-(3-(2-oxo-1,3-oxazinan-3-yl)phenyl)pyrimidin-2-yl)amino)cyclohexyl)cyclohexane-1-carboxamide |
| 462 | 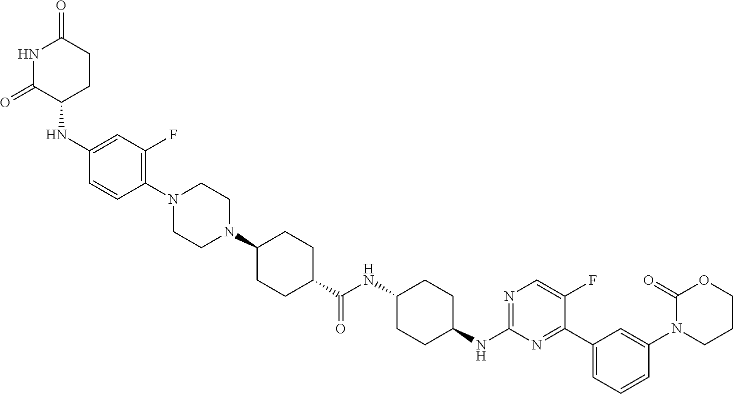 [SMILES: O=C1CC[C@H](Nc2ccc(N3CCN([C@H]4CC[C@H](C(=O)N[C@H]5CC[C@H](Nc6ncc(F)c(-c7cccc(N8CCCOC8=O)c7)n6)CC5)CC4)CC3)c(F)c2)C(=O)N1] |
| | trans-(1S,4R)-4-(4-(4-(((S)-2,6-dioxopiperidin-3-yl)amino)-2-fluorophenyl)piperazin-1-yl)-N-((1r,4R)-4-((5-fluoro- |
| | 4-(3-(2-oxo-1,3-oxazinan-3-yl)phenyl)pyrimidin-2-yl)amino)cyclohexyl)cyclohexane-1-carboxamide |
| 463 | 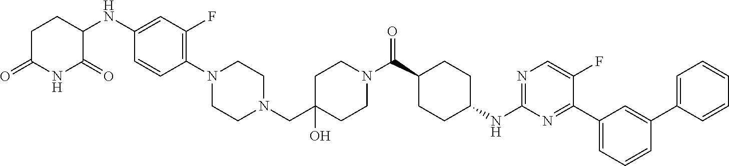 [SMILES: O=C1CCC(Nc2ccc(N3CCN(CC4(O)CCN(C(=O)[C@H]5CC[C@H](Nc6ncc(F)c(-c7cccc(-c8ccccc8)c7)n6)CC5)CC4)CC3)c(F)c2)C(=O)N1] |
| | trans-3-[3-fluoro-4-[4-[[1-[4-[[5-fluoro-4-(3-phenylphenyl)pyrimidin-2-yl]amino]cyclohexanecarbonyl]-4-hydroxy- |
| | 4-piperidyl]methyl]piperazin-1-yl]anilino]piperidine-2,6-dione |
| 464 | 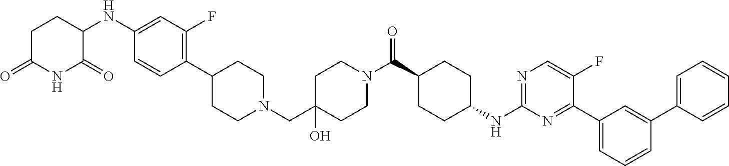 [SMILES: O=C1CCC(Nc2ccc(C3CCN(CC4(O)CCN(C(=O)[C@H]5CC[C@H](Nc6ncc(F)c(-c7cccc(-c8ccccc8)c7)n6)CC5)CC4)CC3)c(F)c2)C(=O)N1] |
| | 3-((4-(1-((1-(trans-4-((4-([1,1′-biphenyl]-3-yl)-5-fluoropyrimidin-2-yl)amino)cyclohexane-1-carbonyl)-4-hydroxy- |
| | piperidin-4-yl)methyl)piperidin-4-yl)-3-fluorophenyl)amino)piperidine-2,6-dione |
| 465 | 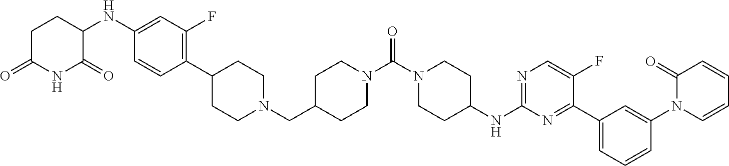 [SMILES: O=C1CCC(Nc2ccc(C3CCN(CC4CCN(C(=O)N5CCC(Nc6ncc(F)c(-c7cccc(-n8ccccc8=O)c7)n6)CC5)CC4)CC3)c(F)c2)C(=O)N1] |
| | 3-((3-fluoro-4-(1-((1-(4-((5-fluoro-4-(3-(2-oxopyridin-1(2H)-yl)phenyl)pyrimidin-2-yl)amino)piperidine-1- |
| | carbonyl)piperidin-4-yl)methyl)piperidin-4-yl)phenyl)amino)piperidine-2,6-dione |
| 466 | 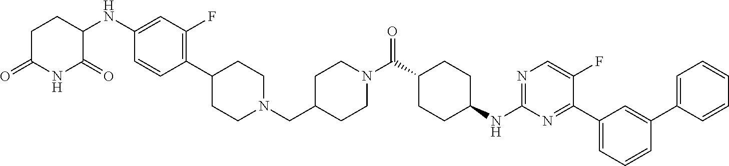 [SMILES: O=C1CCC(Nc2ccc(C3CCN(CC4CCN(C(=O)[C@H]5CC[C@H](Nc6ncc(F)c(-c7cccc(-c8ccccc8)c7)n6)CC5)CC4)CC3)c(F)c2)C(=O)N1] |
| | 3-((4-(1-((1-(trans-4-((4-([1,1′-biphenyl]-3-yl)-5-fluoropyrimidin-2-yl)amino)cyclohexane-1-carbonyl)piperidin-4-yl)- |
| | methyl)piperidin-4-yl)-3-fluorophenyl)amino)piperidine-2,6-dione |
| 467 | 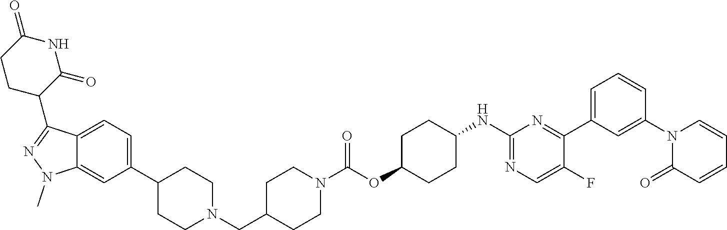 [SMILES: Cn1nc(C2CCC(=O)NC2=O)c2ccc(C3CCN(CC4CCN(C(=O)O[C@H]5CC[C@H](Nc6ncc(F)c(-c7cccc(-n8ccccc8=O)c7)n6)CC5)CC4)CC3)cc21] |
| | trans-4-((5-fluoro-4-(3-(2-oxopyridin-1(2H)-yl)phenyl)pyrimidin-2-yl)amino)cyclohexyl 4-((4-(3-(2,6-dioxopiperidin- |
| | 3-yl)-1-methyl-1H-indazol-6-yl)piperidin-1-yl)methyl)piperidine-1-carboxylate |
| 468 | 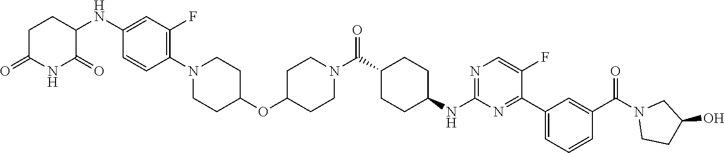 [SMILES: O=C1CCC(Nc2ccc(N3CCC(OC4CCN(C(=O)[C@H]5CC[C@H](Nc6ncc(F)c(-c7cccc(C(=O)N8CC[C@H](O)C8)c7)n6)CC5)CC4)CC3)c(F)c2)C(=O)N1] |
| | trans-3-((3-fluoro-4-(1-((1-(4-((5-fluoro-4-(3-((S)-3-hydroxypyrrolidine-1-carbonyl)phenyl)pyrimidin-2-yl)amino)- |
| | cyclohexane-1-carbonyl)piperidin-4-yl)methyl)piperidin-4-yl)phenyl)amino)piperidine-2,6-dione |
| 469 | 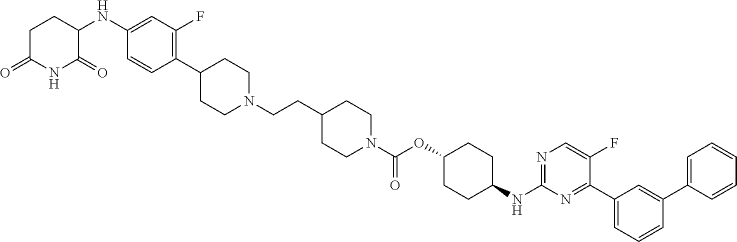 [SMILES: O=C1CCC(Nc2ccc(C3CCN(CCC4CCN(C(=O)O[C@H]5CC[C@H](Nc6ncc(F)c(-c7cccc(-c8ccccc8)c7)n6)CC5)CC4)CC3)c(F)c2)C(=O)N1] |
| | trans-4-((4-([1,1′-biphenyl]-3-yl)-5-fluoropyrimidin-2-yl)amino)cyclohexyl 4-(2-(4-(4-((2,6-dioxopiperidin-3-yl)amino)- |
| | 2-fluorophenyl)piperidin-1-yl)ethyl)piperidine-1-carboxylate |
| 470 | 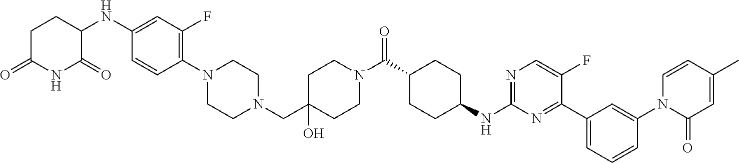 [SMILES: Cc1ccn(-c2cccc(-c3nc(N[C@H]4CC[C@H](C(=O)N5CCC(O)(CN6CCN(c7ccc(NC8CCC(=O)NC8=O)cc7F)CC6)CC5)CC4)ncc3F)c2)c(=O)c1] |
| | trans-3-((3-fluoro-4-(4-((1-(4-((5-fluoro-4-(3-(4-methyl-2-oxopyridin-1(2H)-yl)phenyl)pyrimidin-2-yl)amino)cyclo- |
| | hexane-1-carbonyl)-4-hydroxypiperidin-4-yl)methyl)piperazin-1-yl)phenyl)amino)piperidine-2,6-dione |
| 471 | 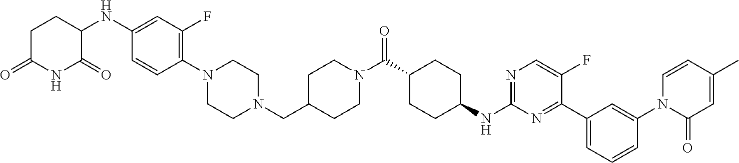 [SMILES: Cc1ccn(-c2cccc(-c3nc(N[C@H]4CC[C@H](C(=O)N5CCC(CN6CCN(c7ccc(NC8CCC(=O)NC8=O)cc7F)CC6)CC5)CC4)ncc3F)c2)c(=O)c1] |
| | trans-3-((3-fluoro-4-(4-((1-(4-((5-fluoro-4-(3-(4-methyl-2-oxopyridin-1(2H)-yl)phenyl)pyrimidin-2-yl)amino)cyclo- |
| | hexane-1-carbonyl)piperidin-4-yl)methyl)piperazin-1-yl)phenyl)amino)piperidine-2,6-dione |
| 472 | 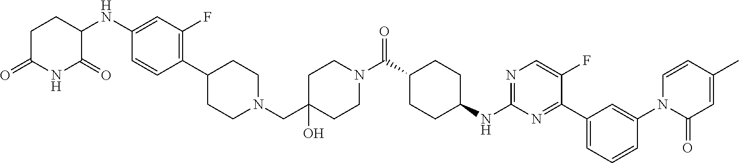 [SMILES: Cc1ccn(-c2cccc(-c3nc(N[C@H]4CC[C@H](C(=O)N5CCC(O)(CN6CCC(c7ccc(NC8CCC(=O)NC8=O)cc7F)CC6)CC5)CC4)ncc3F)c2)c(=O)c1] |
| | trans-3-[3-fluoro-4-[1-[[1-[4-[[5-fluoro-4-[3-(4-methyl-2-oxo-1-pyridyl)phenyl]pyrimidin-2-yl]amino]cyclohexane- |
| | carbonyl]-4-hydroxy-4-piperidyl]methyl]-4-piperidyl]anilino]piperidine-2,6-dione |
| 473 | 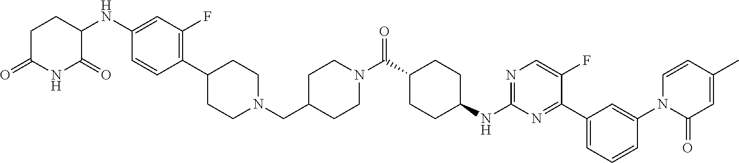 [SMILES: Cc1ccn(-c2cccc(-c3nc(N[C@H]4CC[C@H](C(=O)N5CCC(CN6CCC(c7ccc(NC8CCC(=O)NC8=O)cc7F)CC6)CC5)CC4)ncc3F)c2)c(=O)c1] |
| | trans-3-((3-fluoro-4-(1-((1-(4-((5-fluoro-4-(3-(4-methyl-2-oxopyridin-1(2H)-yl)phenyl)pyrimidin-2-yl)amino)cyclo- |
| | hexane-1-carbonyl)piperidin-4-yl)methyl)piperidin-4-yl)phenyl)amino)piperidine-2,6-dione |
| 474 | 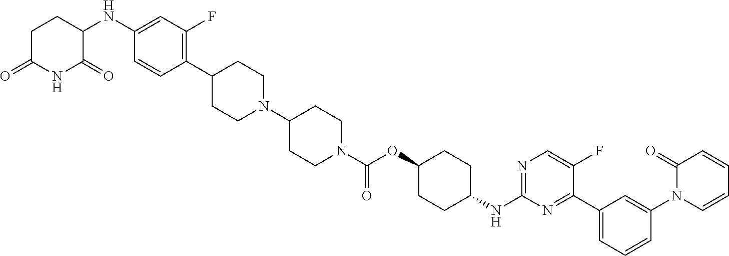 [SMILES: O=C1CCC(Nc2ccc(C3CCN(C4CCN(C(=O)O[C@H]5CC[C@H](Nc6ncc(F)c(-c7cccc(-n8ccccc8=O)c7)n6)CC5)CC4)CC3)c(F)c2)C(=O)N1] |
| | trans-4-((5-fluoro-4-(3-(2-oxopyridin-1(2H)-yl)phenyl)pyrimidin-2-yl)amino)cyclohexyl 4-(4-((2,6-dioxopiperidin-3- |
| | yl)amino)-2-fluorophenyl)-[1,4′-bipiperidine]-1′-carboxylate |
| 475 | 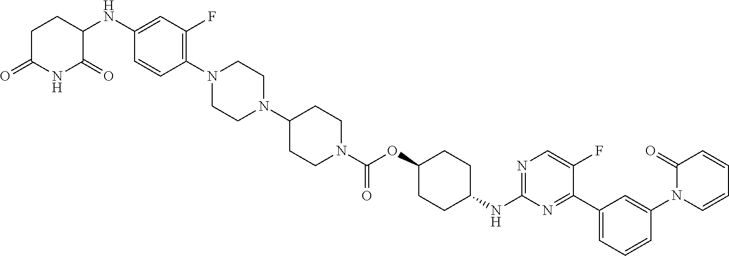 [SMILES: O=C1CCC(Nc2ccc(N3CCN(C4CCN(C(=O)O[C@H]5CC[C@H](Nc6ncc(F)c(-c7cccc(-n8ccccc8=O)c7)n6)CC5)CC4)CC3)c(F)c2)C(=O)N1] |
| | trans-4-((5-fluoro-4-(3-(2-oxopyridin-1(2H)-yl)phenyl)pyrimidin-2-yl)amino)cyclohexyl 4-(4-(4-((2,6-dioxopiperidin-3- |
| | yl)amino)-2-fluorophenyl)piperazin-1-yl)piperidine-1-carboxylate |
| 476 | 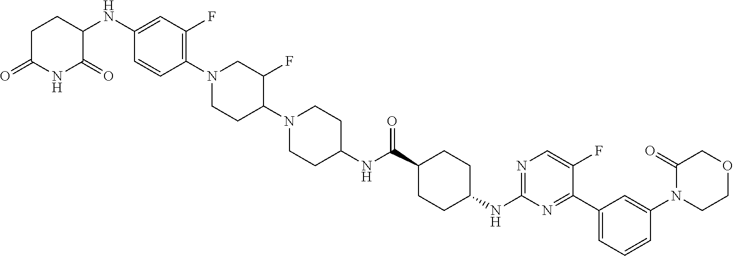 [SMILES: O=C1CCC(Nc2ccc(N3CCC(N4CCC(NC(=O)[C@H]5CC[C@H](Nc6ncc(F)c(-c7cccc(N8CCOCC8=O)c7)n6)CC5)CC4)C(F)C3)c(F)c2)C(=O)N1] |
| | trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(3- |
| | oxomorpholino)phenyl)pyrimidin-2-yl)amino)cyclohexane-1-carboxamide |
| 477 | 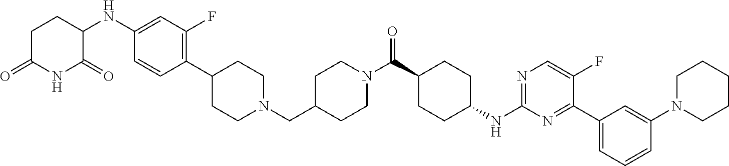 [SMILES: O=C1CCC(Nc2ccc(C3CCN(CC4CCN(C(=O)[C@H]5CC[C@H](Nc6ncc(F)c(-c7cccc(N8CCCCC8)c7)n6)CC5)CC4)CC3)c(F)c2)C(=O)N1] |
| | trans-3-((3-fluoro-4-(1-((1-(trans-4-((5-fluoro-4-(3-(piperidin-1-yl)phenyl)pyrimidin-2-yl)amino)cyclohexane-1- |
| | carbonyl)piperidin-4-yl)methyl)piperidin-4-yl)phenyl)amino)piperidine-2,6-dione |
| 478 | 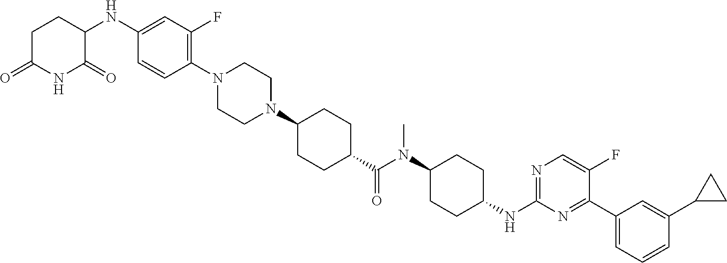 [SMILES: CN(C(=O)[C@H]1CC[C@H](N2CCN(c3ccc(NC4CCC(=O)NC4=O)cc3F)CC2)CC1)[C@H]1CC[C@H](Nc2ncc(F)c(-c3cccc(C4CC4)c3)n2)CC1] |
| | trans-N-((trans)-4-((4-(3-cyclopropylphenyl)-5-fluoropyrimidin-2-yl)amino)cyclohexyl)-4-(4-(4-((2,6-dioxopiperidin- |
| | 3-yl)amino)-2-fluorophenyl)piperazin-1-yl)-N-methylcyclohexane-1-carboxamide |
| 479 | 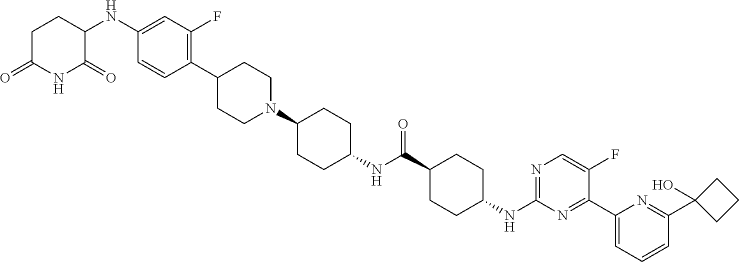 [SMILES: O=C1CCC(Nc2ccc(C3CCN([C@H]4CC[C@H](NC(=O)[C@H]5CC[C@H](Nc6ncc(F)c(-c7cccc(C8(O)CCC8)n7)n6)CC5)CC4)CC3)c(F)c2)C(=O)N1] |
| | trans-N-((trans)-4-(4-(4-((2,6-dioxopiperidin-3-yl)amino)-2-fluorophenyl)piperidin-1-yl)cyclohexyl)-4-((5-fluoro- |
| | 4-(6-(1-hydroxycyclobutyl)pyridin-2-yl)pyrimidin-2-yl)amino)cyclohexane-1-carboxamide |
| 480 | 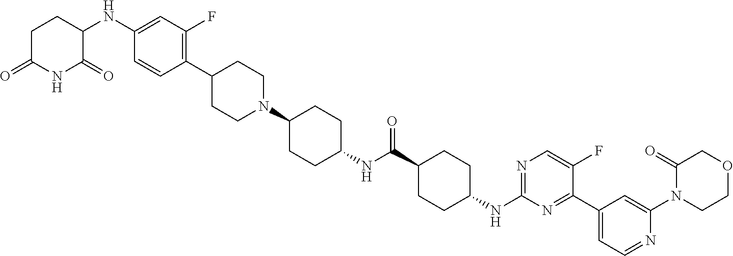 [SMILES: O=C1CCC(Nc2ccc(C3CCN([C@H]4CC[C@H](NC(=O)[C@H]5CC[C@H](Nc6ncc(F)c(-c7ccnc(N8CCOCC8=O)c7)n6)CC5)CC4)CC3)c(F)c2)C(=O)N1] |
| | trans-N-((trans)-4-(4-(4-((2,6-dioxopiperidin-3-yl)amino)-2-fluorophenyl)piperidin-1-yl)cyclohexyl)-4-((5-fluoro- |
| | 4-(2-(3-oxomorpholino)pyridin-4-yl)pyrimidin-2-yl)amino)cyclohexane-1-carboxamide |
| 481 | 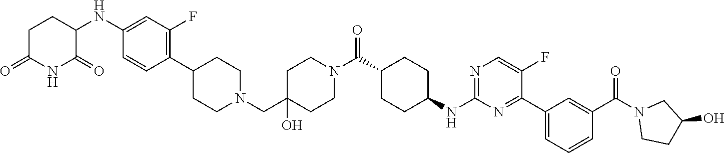 [SMILES: O=C1CCC(Nc2ccc(C3CCN(CC4(O)CCN(C(=O)[C@H]5CC[C@H](Nc6ncc(F)c(-c7cccc(C(=O)N8CC[C@H](O)C8)c7)n6)CC5)CC4)CC3)c(F)c2)C(=O)N1] |
| | trans-3-((3-fluoro-4-(1-((1-(4-((5-fluoro-4-(3-((S)-3-hydroxypyrrolidine-1-carbonyl)phenyl)pyrimidin-2-yl)amino)- |
| | cyclohexane-1-carbonyl)-4-hydroxypiperidin-4-yl)methyl)piperidin-4-yl)phenyl)amino)piperidine-2,6-dione |
| 482 | 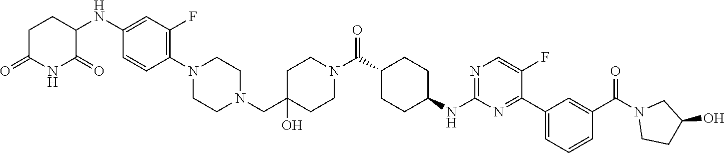 [SMILES: O=C1CCC(Nc2ccc(N3CCN(CC4(O)CCN(C(=O)[C@H]5CC[C@H](Nc6ncc(F)c(-c7cccc(C(=O)N8CC[C@H](O)C8)c7)n6)CC5)CC4)CC3)c(F)c2)C(=O)N1] |
| | trans-3-((3-fluoro-4-(4-((1-(4-((5-fluoro-4-(3-((S)-3-hydroxypyrrolidine-1-carbonyl)phenyl)pyrimidin-2-yl)amino) |
| | cyclohexane-1-carbonyl)-4-hydroxypiperidin-4-yl)methyl)piperazin-1-yl)phenyl)amino)piperidine-2,6-dione |
| 483 | 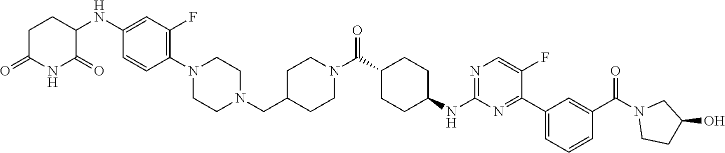 [SMILES: O=C1CCC(Nc2ccc(N3CCN(CC4CCN(C(=O)[C@H]5CC[C@H](Nc6ncc(F)c(-c7cccc(C(=O)N8CC[C@H](O)C8)c7)n6)CC5)CC4)CC3)c(F)c2)C(=O)N1] |
| | trans-3-((3-fluoro-4-(4-((1-(4-((5-fluoro-4-(3-((S)-3-hydroxypyrrolidine-1-carbonyl)phenyl)pyrimidin-2-yl)amino)- |
| | cyclohexane-1-carbonyl)piperidin-4-yl)methyl)piperazin-1-yl)phenyl)amino)piperidine-2,6-dione |
| 484 | 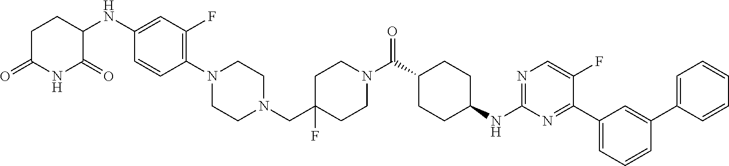 [SMILES: O=C1CCC(Nc2ccc(N3CCN(CC4(F)CCN(C(=O)[C@H]5CC[C@H](Nc6ncc(F)c(-c7cccc(-c8ccccc8)c7)n6)CC5)CC4)CC3)c(F)c2)C(=O)N1] |
| | trans-3-[3-fluoro-4-[4-[[4-fluoro-1-[4-[[5-fluoro-4-(3-phenylphenyl)pyrimidin-2-yl]amino]cyclohexanecarbonyl]- |
| | 4-piperidyl]methyl]piperazin-1-yl]anilino]piperidine-2,6-dione |
| 485 | 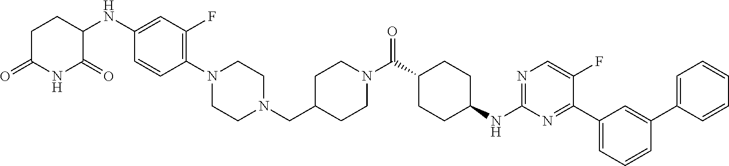 [SMILES: O=C1CCC(Nc2ccc(N3CCN(CC4CCN(C(=O)[C@H]5CC[C@H](Nc6ncc(F)c(-c7cccc(-c8ccccc8)c7)n6)CC5)CC4)CC3)c(F)c2)C(=O)N1] |
| | trans-3-((4-(4-((1-(4-((4-([1,1′-biphenyl]-3-yl)-5-fluoropyrimidin-2-yl)amino)cyclohexane-1-carbonyl)piperidin-4-yl)- |
| | methyl)piperazin-1-yl)-3-fluorophenyl)amino)piperidine-2,6-dione |
| 486 | 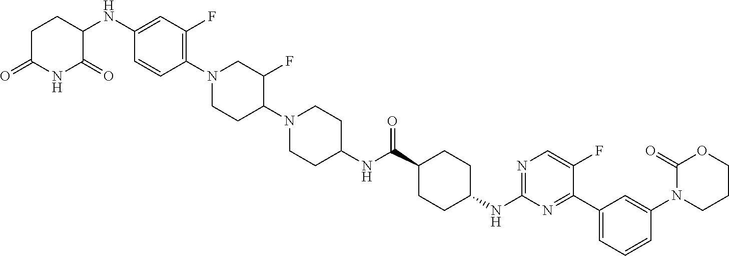 [SMILES: O=C1CCC(Nc2ccc(N3CCC(N4CCC(NC(=O)[C@H]5CC[C@H](Nc6ncc(F)c(-c7cccc(N8CCCOC8=O)c7)n6)CC5)CC4)C(F)C3)c(F)c2)C(=O)N1] |
| | trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(2- |
| | oxo-1,3-oxazinan-3-yl)phenyl)pyrimidin-2-yl)amino)cyclohexane-1-carboxamide |
| 487 | 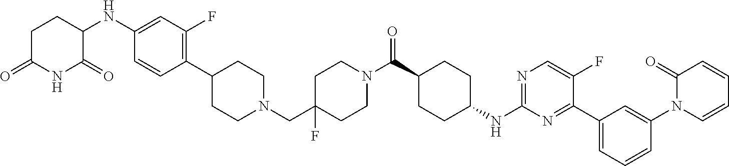 [SMILES: O=C1CCC(Nc2ccc(C3CCN(CC4(F)CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-3-((3-fluoro-4-(1-((4-fluoro-1-(4-((5-fluoro-4-(3-(2-oxopyridin-1(2H)-yl)phenyl)pyrimidin-2-yl)amino)cyclo- |
| | hexane-1-carbonyl)piperidin-4-yl)methyl)piperidin-4-yl)phenyl)amino)piperidine-2,6-dione |
| 488 | 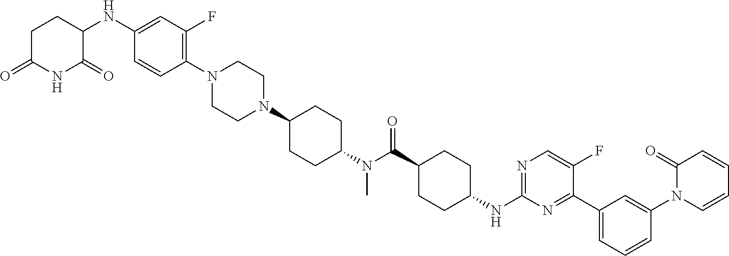 [SMILES: CN(C(=O)[C@H]1CC[C@H](Nc2ncc(F)c(-c3cccc(-n4ccccc4=O)c3)n2)CC1)[C@H]1CC[C@H](N2CCN(c3ccc(NC4CCC(=O)NC4=O)cc3F)CC2)CC1] |
| | trans-(1r,4R)-N-((1r,4R)-4-(4-(4-((2,6-dioxopiperidin-3-yl)amino)-2-fluorophenyl)piperazin-1-yl)cyclohexyl)-4-((5- |
| | fluoro-4-(3-(2-oxopyridin-1(2H)-yl)phenyl)pyrimidin-2-yl)amino)-N-methylcyclohexane-1-carboxamide |
| 489 | 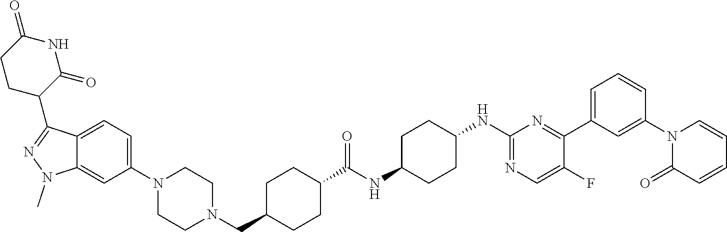 [SMILES: Cn1nc(C2CCC(=O)NC2=O)c2ccc(N3CCN(C[C@H]4CC[C@H](C(=O)N[C@H]5CC[C@H](Nc6ncc(F)c(-c7cccc(-n8ccccc8=O)c7)n6)CC5)CC4)CC3)cc21] |
| | trans-(1r,4R)-4-((4-(3-(2,6-dioxopiperidin-3-yl)-1-methyl-1H-indazol-6-yl)piperazin-1-yl)methyl)-N-((1r,4R)-4-((5- |
| | fluoro-4-(3-(2-oxopyridin-1(2H)-yl)phenyl)pyrimidin-2-yl)amino)cyclohexyl)cyclohexane-1-carboxamide |
| 490 | 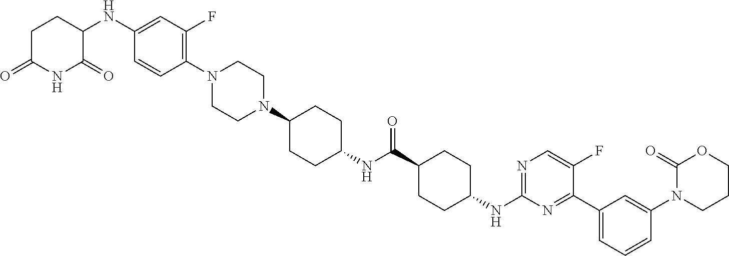 [SMILES: O=C1CCC(Nc2ccc(N3CCN([C@H]4CC[C@H](NC(=O)[C@H]5CC[C@H](Nc6ncc(F)c(-c7cccc(N8CCCOC8=O)c7)n6)CC5)CC4)CC3)c(F)c2)C(=O)N1] |
| | trans-N-(trans-4-(4-(4-((2,6-dioxopiperidin-3-yl)amino)-2-fluorophenyl)piperazin-1-yl)cyclohexyl)-4-((5-fluoro- |
| | 4-(3-(2-oxo-1,3-oxazinan-3-yl)phenyl)pyrimidin-2-yl)amino)cyclohexane-1-carboxamide |
| 491 | 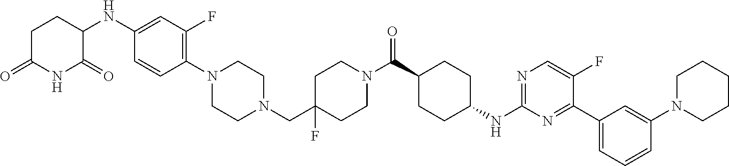 [SMILES: O=C1CCC(Nc2ccc(N3CCN(CC4(F)CCN(C(=O)[C@H]5CC[C@H](Nc6ncc(F)c(-c7cccc(N8CCCCC8)c7)n6)CC5)CC4)CC3)c(F)c2)C(=O)N1] |
| | trans-3-((3-fluoro-4-(4-((4-fluoro-1-(4-((5-fluoro-4-(3-(piperidin-1-yl)phenyl)pyrimidin-2-yl)amino)cyclohexane-1- |
| | carbonyl)piperidin-4-yl)methyl)piperazin-1-yl)phenyl)amino)piperidine-2,6-dione |
| 492 | 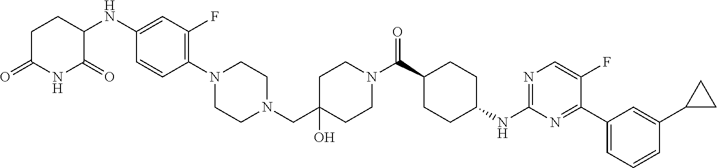 [SMILES: O=C1CCC(Nc2ccc(N3CCN(CC4(O)CCN(C(=O)[C@H]5CC[C@H](Nc6ncc(F)c(-c7cccc(C8CC8)c7)n6)CC5)CC4)CC3)c(F)c2)C(=O)N1] |
| | trans-3-((4-(4-((1-(4-((4-(3-cyclopropylphenyl)-5-fluoropyrimidin-2-yl)amino)cyclohexane-1-carbonyl)-4-hydroxy- |
| | piperidin-4-yl)methyl)piperazin-1-yl)-3-fluorophenyl)amino)piperidine-2,6-dione |
| 493 | 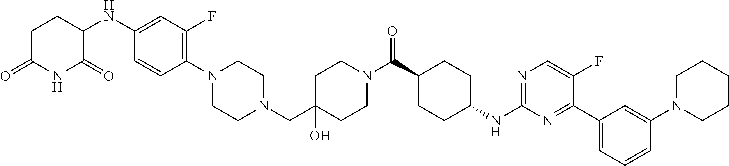 [SMILES: O=C1CCC(Nc2ccc(N3CCN(CC4(O)CCN(C(=O)[C@H]5CC[C@H](Nc6ncc(F)c(-c7cccc(N8CCCCC8)c7)n6)CC5)CC4)CC3)c(F)c2)C(=O)N1] |
| | trans-3-[3-fluoro-4-[4-[[1-[4-[5-fluoro-4-[3-(1-piperidyl)phenyl]pyrimidin-2-yl]amino]cyclohexanecarbonyl]-4- |
| | hydroxy-4-piperidyl]methyl]piperazin-1-yl]anilino]piperidine-2,6-dione |
| 494 | 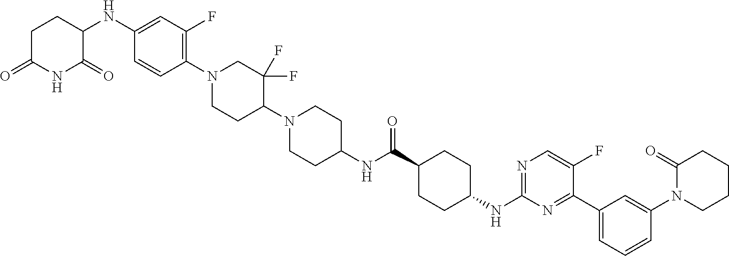 [SMILES: O=C1CCC(Nc2ccc(N3CCC(N4CCC(NC(=O)[C@H]5CC[C@H](Nc6ncc(F)c(-c7cccc(N8CCCCC8=O)c7)n6)CC5)CC4)C(F)(F)C3)c(F)c2)C(=O)N1] |
| | trans-N-(1′-(4-((2,6-dioxopiperidin-3-yl)amino)-2-fluorophenyl)-3′,3′-difluoro-[1,4′-bipiperidin]-4-yl)-4-((5-fluoro- |
| | 4-(3-(2-oxopiperidin-1-yl)phenyl)pyrimidin-2-yl)amino)cyclohexane-1-carboxamide |
| 495 | 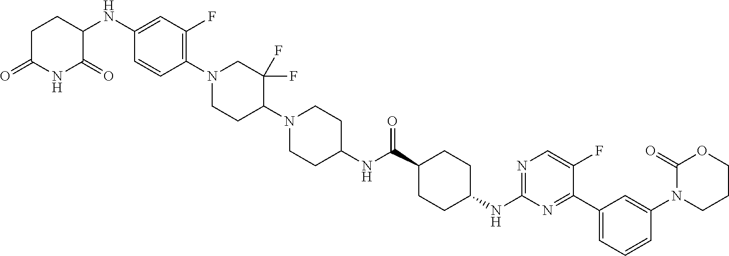 [SMILES: O=C1CCC(Nc2ccc(N3CCC(N4CCC(NC(=O)[C@H]5CC[C@H](Nc6ncc(F)c(-c7cccc(N8CCCOC8=O)c7)n6)CC5)CC4)C(F)(F)C3)c(F)c2)C(=O)N1] |
| | trans-N-(1′-(4-((2,6-dioxopiperidin-3-yl)amino)-2-fluorophenyl)-3′,3′-difluoro-[1,4′-bipiperidin]-4-yl)-4-((5-fluoro-4- |
| | (3-(2-oxo-1,3-oxazinan-3-yl)phenyl)pyrimidin-2-yl)amino)cyclohexane-1-carboxamide |
| 496 | 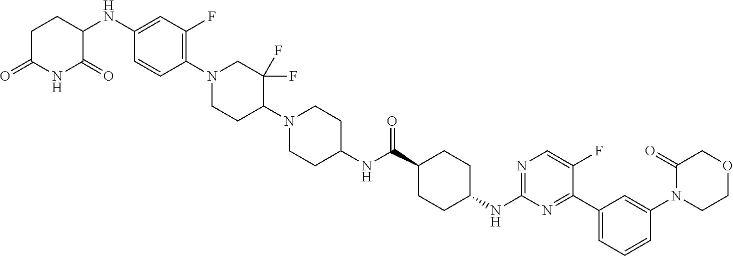 [SMILES: O=C1CCC(Nc2ccc(N3CCC(N4CCC(NC(=O)[C@H]5CC[C@H](Nc6ncc(F)c(-c7cccc(N8CCOCC8=O)c7)n6)CC5)CC4)C(F)(F)C3)c(F)c2)C(=O)N1] |
| | trans-N-(1′-(4-((2,6-dioxopiperidin-3-yl)amino)-2-fluorophenyl)-3′,3′-difluoro-[1,4′-bipiperidin]-4-yl)-4-((5-fluoro-4- |
| | (3-(3-oxomorpholino)phenyl)pyrimidin-2-yl)amino)cyclohexane-1-carboxamide |
| 497 | 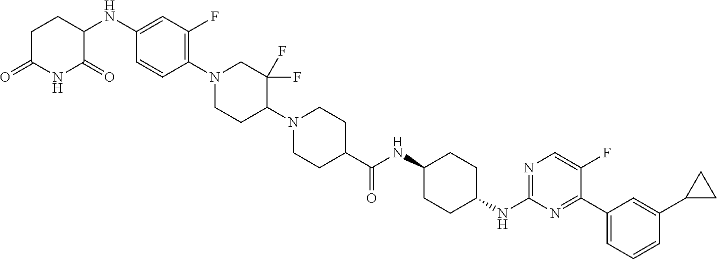 [SMILES: O=C1CCC(Nc2ccc(N3CCC(N4CCC(C(=O)N[C@H]5CC[C@H](Nc6ncc(F)c(-c7cccc(C8CC8)c7)n6)CC5)CC4)C(F)(F)C3)c(F)c2)C(=O)N1] |
| | trans-N-(4-((4-(3-cyclopropylphenyl)-5-fluoropyrimidin-2-yl)amino)cyclohexyl)-1′-(4-((2,6-dioxopiperidin-3-yl)- |
| | amino)-2-fluorophenyl)-3′,3′-difluoro-[1,4′-bipiperidine]-4-carboxamide |
| 498 | 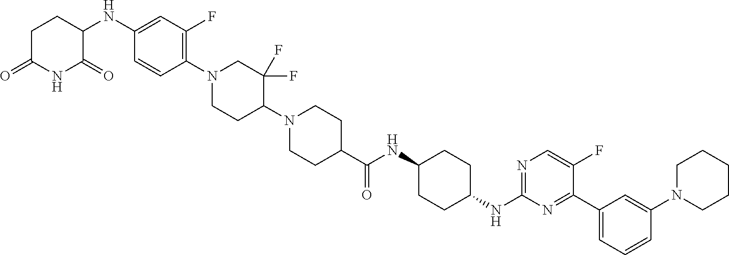 [SMILES: O=C1CCC(Nc2ccc(N3CCC(N4CCC(C(=O)N[C@H]5CC[C@H](Nc6ncc(F)c(-c7cccc(N8CCCCC8)c7)n6)CC5)CC4)C(F)(F)C3)c(F)c2)C(=O)N1] |
| | trans-1′-(4-((2,6-dioxopiperidin-3-yl)amino)-2-fluorophenyl)-3′,3′-difluoro-N-(4-((5-fluoro-4-(3-(piperidin-1-yl)phen- |
| | yl)pyrimidin-2-yl)amino)cyclohexyl)-[1,4′-bipiperidine]-4-carboxamide |
| 499 | 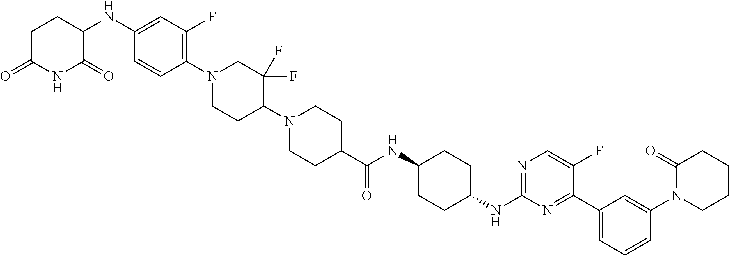 [SMILES: O=C1CCC(Nc2ccc(N3CCC(N4CCC(C(=O)N[C@H]5CC[C@H](Nc6ncc(F)c(-c7cccc(N8CCCCC8=O)c7)n6)CC5)CC4)C(F)(F)C3)c(F)c2)C(=O)N1] |
| | trans-1′-(4-((2,6-dioxopiperidin-3-yl)amino)-2-fluorophenyl)-3′,3′-difluoro-N-(4-((5-fluoro-4-(3-(2-oxopiperidin-1- |
| | yl)phenyl)pyrimidin-2-yl)amino)cyclohexyl)-[1,4′-bipiperidine]-4-carboxamide |
| 500 | 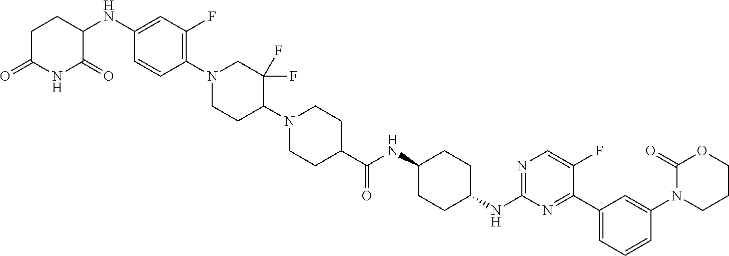 [SMILES: O=C1CCC(Nc2ccc(N3CCC(N4CCC(C(=O)N[C@H]5CC[C@H](Nc6ncc(F)c(-c7cccc(N8CCCOC8=O)c7)n6)CC5)CC4)C(F)(F)C3)c(F)c2)C(=O)N1] |
| | trans-1′-(4-((2,6-dioxopiperidin-3-yl)amino)-2-fluorophenyl)-3′,3′-difluoro-N-(4-((5-fluoro-4-(3-(2-oxo-1,3-oxazinan- |
| | 3-yl)phenyl)pyrimidin-2-yl)amino)cyclohexyl)-[1,4′-bipiperidine]-4-carboxamide |
| 501 | 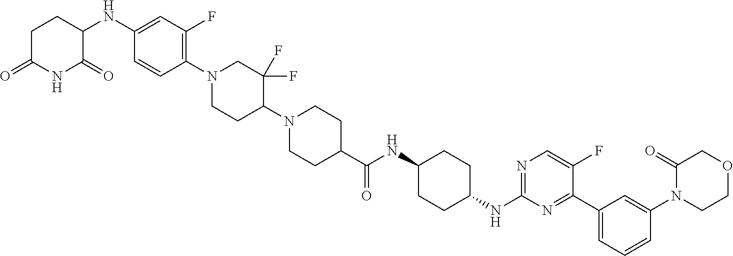 [SMILES: O=C1CCC(Nc2ccc(N3CCC(N4CCC(C(=O)N[C@H]5CC[C@H](Nc6ncc(F)c(-c7cccc(N8CCOCC8=O)c7)n6)CC5)CC4)C(F)(F)C3)c(F)c2)C(=O)N1] |
| | trans-1′-(4-((2,6-dioxopiperidin-3-yl)amino)-2-fluorophenyl)-3′,3′-difluoro-N-(4-((5-fluoro-4-(3-(3-oxomorpholino) |
| | phenyl)pyrimidin-2-yl)amino)cyclohexyl)-[1,4′-bipiperidine]-4-carboxamide |
| 502 | 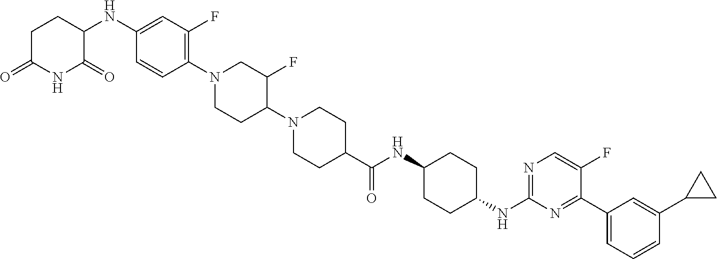 [SMILES: O=C1CCC(Nc2ccc(N3CCC(N4CCC(C(=O)N[C@H]5CC[C@H](Nc6ncc(F)c(-c7cccc(C8CC8)c7)n6)CC5)CC4)C(F)C3)c(F)c2)C(=O)N1] |
| | N-((1r,4r)-4-((4-(3-cyclopropylphenyl)-5-fluoropyrimidin-2-yl)amino)cyclohexyl)-1′-(4-((2,6-dioxopiperidin-3-yl)- |
| | amino)-2-fluorophenyl)-3′-fluoro-[1,4′-bipiperidine]-4-carboxamide |
| 503 | 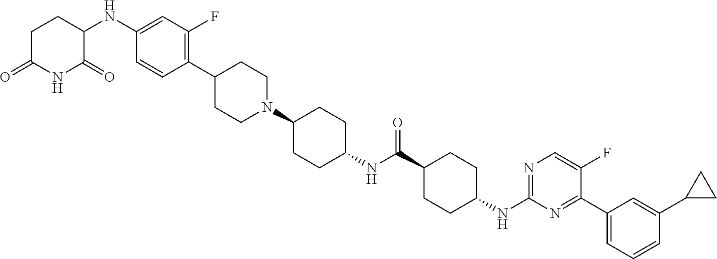 [SMILES: O=C1CCC(Nc2ccc(C3CCN([C@H]4CC[C@H](NC(=O)[C@H]5CC[C@H](Nc6ncc(F)c(-c7cccc(C8CC8)c7)n6)CC5)CC4)CC3)c(F)c2)C(=O)N1] |
| | trans-4-((4-(3-cyclopropylphenyl)-5-fluoropyrimidin-2-yl)amino)-N-(trans-4-(4-(4-((2,6-dioxopiperidin-3-yl)amino)- |
| | 2-fluorophenyl)piperidin-1-yl)cyclohexyl)cyclohexane-1-carboxamide |
| 504 | 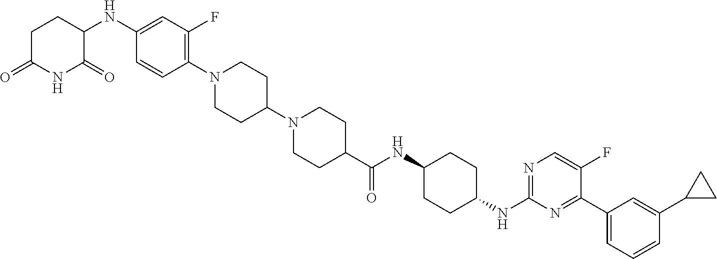 [SMILES: O=C1CCC(Nc2ccc(N3CCC(N4CCC(C(=O)N[C@H]5CC[C@H](Nc6ncc(F)c(-c7cccc(C8CC8)c7)n6)CC5)CC4)CC3)c(F)c2)C(=O)N1] |
| | trans-N-(4-((4-(3-cyclopropylphenyl)-5-fluoropyrimidin-2-yl)amino)cyclohexyl)-1′-(4-((2,6-dioxopiperidin-3-yl)- |
| | amino)-2-fluorophenyl)-[1,4′-bipiperidine]-4-carboxamide |
| 505 | 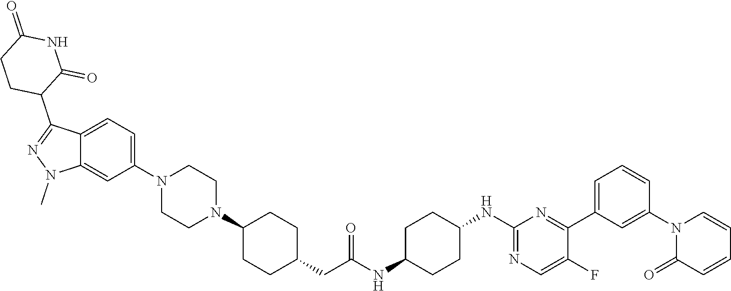 [SMILES: Cn1nc(C2CCC(=O)NC2=O)c2ccc(N3CCN([C@H]4CC[C@H](CC(=O)N[C@H]5CC[C@H](Nc6ncc(F)c(-c7cccc(-n8ccccc8=O)c7)n6)CC5)CC4)CC3)cc21] |
| | trans-2-((1r,4R)-4-(4-(3-(2,6-dioxopiperidin-3-yl)-1-methyl-1H-indazol-6-yl)piperazin-1-yl)cyclohexyl)-N-((1r,4R)-4- |
| | ((5-fluoro-4-(3-(2-oxopyridin-1(2H)-yl)phenyl)pyrimidin-2-yl)amino)cyclohexyl)acetamide |
| 506 | 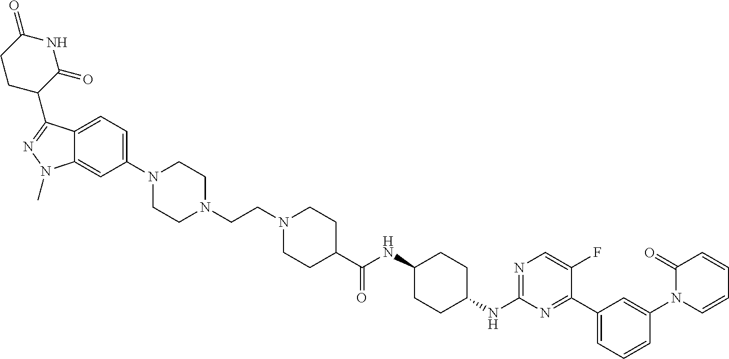 [SMILES: Cn1nc(C2CCC(=O)NC2=O)c2ccc(N3CCN(CCN4CCC(C(=O)N[C@H]5CC[C@H](Nc6ncc(F)c(-c7cccc(-n8ccccc8=O)c7)n6)CC5)CC4)CC3)cc21] |
| | rac-1-(2-(4-(3-(2,6-dioxopiperidin-3-yl)-1-methyl-1H-indazol-6-yl)piperazin-1-yl)ethyl)-N-((1r,4r)-4-((5-fluoro-4-(3- |
| | (2-oxopyridin-1(2H)-yl)phenyl)pyrimidin-2-yl)amino)cyclohexyl)piperidine-4-carboxamide |
| 507 | 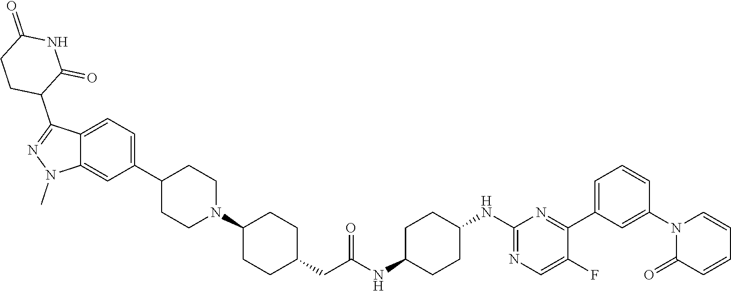 [SMILES: Cn1nc(C2CCC(=O)NC2=O)c2ccc(C3CCN([C@H]4CC[C@H](CC(=O)N[C@H]5CC[C@H](Nc6ncc(F)c(-c7cccc(-n8ccccc8=O)c7)n6)CC5)CC4)CC3)cc21] |
| | trans-2-((1r,4R)-4-(4-(3-(2,6-dioxopiperidin-3-yl)-1-methyl-1H-indazol-6-yl)piperidin-1-yl)cyclohexyl)-N-((1r,4R)- |
| | 4-((5-fluoro-4-(3-(2-oxopyridin-1(2H)-yl)phenyl)pyrimidin-2-yl)amino)cyclohexyl)acetamide |
| 508 | 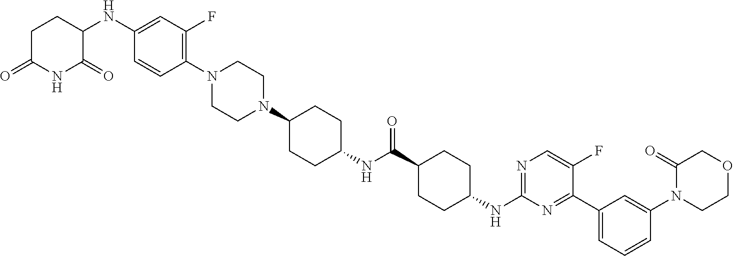 [SMILES: O=C1CCC(Nc2ccc(N3CCN([C@H]4CC[C@H](NC(=O)[C@H]5CC[C@H](Nc6ncc(F)c(-c7cccc(N8CCOCC8=O)c7)n6)CC5)CC4)CC3)c(F)c2)C(=O)N1] |
| | rac-(1r,4R)-N-((1R,4R)-4-(4-(4-(((R)-2,6-dioxopiperidin-3-yl)amino)-2-fluorophenyl)piperazin-1-yl)cyclohexyl)-4- |
| | ((5-fluoro-4-(3-(3-oxomorpholino)phenyl)pyrimidin-2-yl)amino)cyclohexane-1-carboxamide |
| 509 | 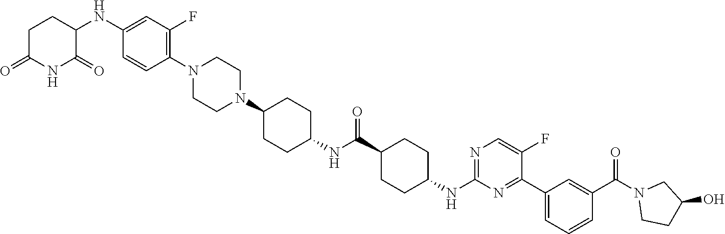 [SMILES: O=C1CCC(Nc2ccc(N3CCN([C@H]4CC[C@H](NC(=O)[C@H]5CC[C@H](Nc6ncc(F)c(-c7cccc(C(=O)N8CC[C@H](O)C8)c7)n6)CC5)CC4)CC3)c(F)c2)C(=O)N1] |
| | trans-N-(trans-4-(4-(4-((2,6-dioxopiperidin-3-yl)amino)-2-fluorophenyl)piperazin-1-yl)cyclohexyl)-4-((5-fluoro-4- |
| | (3-((S)-3-hydroxypyrrolidine-1-carbonyl)phenyl)pyrimidin-2-yl)amino)cyclohexane-1-carboxamide |
| 510 | 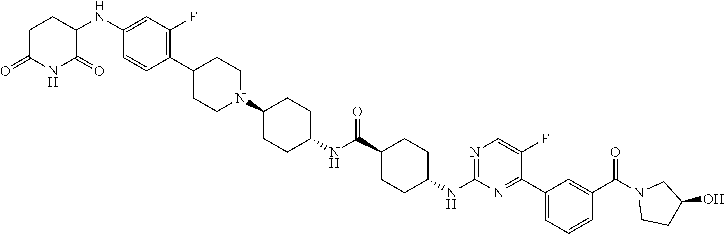 [SMILES: O=C1CCC(Nc2ccc(C3CCN([C@H]4CC[C@H](NC(=O)[C@H]5CC[C@H](Nc6ncc(F)c(-c7cccc(C(=O)N8CC[C@H](O)C8)c7)n6)CC5)CC4)CC3)c(F)c2)C(=O)N1] |
| | trans-N-(trans-4-(4-(4-((2,6-dioxopiperidin-3-yl)amino)-2-fluorophenyl)piperidin-1-yl)cyclohexyl)-4-((5-fluoro-4- |
| | (3-((S)-3-hydroxypyrrolidine-1-carbonyl)phenyl)pyrimidin-2-yl)amino)cyclohexane-1-carboxamide |
| 511 | 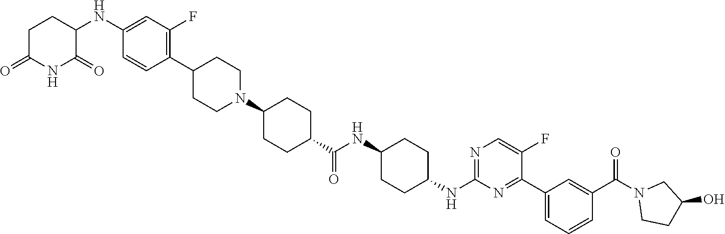 [SMILES: O=C1CCC(Nc2ccc(C3CCN([C@H]4CC[C@H](C(=O)N[C@H]5CC[C@H](Nc6ncc(F)c(-c7cccc(C(=O)N8CC[C@H](O)C8)c7)n6)CC5)CC4)CC3)c(F)c2)C(=O)N1] |
| | trans-4-(4-(4-((2,6-dioxopiperidin-3-yl)amino)-2-fluorophenyl)piperidin-1-yl)-N-(trans-4-((5-fluoro-4-(3-((S)-3- |
| | hydroxypyrrolidine-1-carbonyl)phenyl)pyrimidin-2-yl)amino)cyclohexyl)cyclohexane-1-carboxamide |
| 512 | 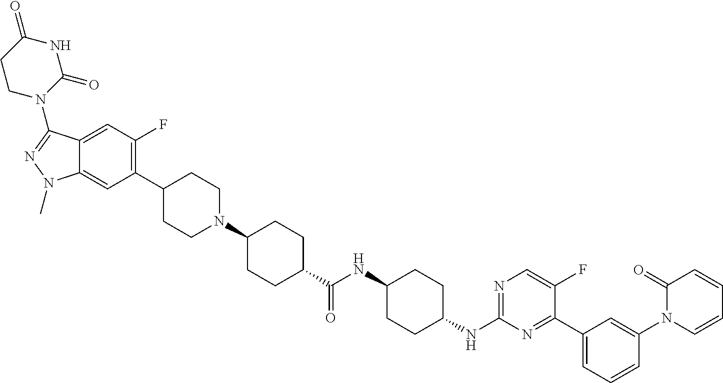 [SMILES: Cn1nc(N2CCC(=O)NC2=O)c2cc(F)c(C3CCN([C@H]4CC[C@H](C(=O)N[C@H]5CC[C@H](Nc6ncc(F)c(-c7cccc(-n8ccccc8=O)c7)n6)CC5)CC4)CC3)cc21] |
| | trans-4-(4-(3-(2,4-dioxotetrahydropyrimidin-1(2H)-yl)-5-fluoro-1-methyl-1H-indazol-6-yl)piperidin-1-yl)-N-(trans- |
| | 4-((5-fluoro-4-(3-(2-oxopyridin-1(2H)-yl)phenyl)pyrimidin-2-yl)amino)cyclohexyl)cyclohexane-1-carboxamide |
| 513 | 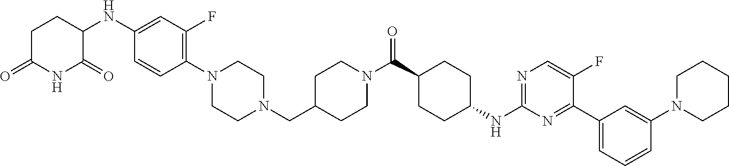 [SMILES: O=C1CCC(Nc2ccc(N3CCN(CC4CCN(C(=O)[C@H]5CC[C@H](Nc6ncc(F)c(-c7cccc(N8CCCCC8)c7)n6)CC5)CC4)CC3)c(F)c2)C(=O)N1] |
| | trans-3-((3-fluoro-4-(4-((1-(4-((5-fluoro-4-(3-(piperidin-1-yl)phenyl)pyrimidin-2-yl)amino)cyclohexane-1-carbonyl) |
| | piperidin-4-yl)methyl)piperazin-1-yl)phenyl)amino)piperidine-2,6-dione |
| 514 | 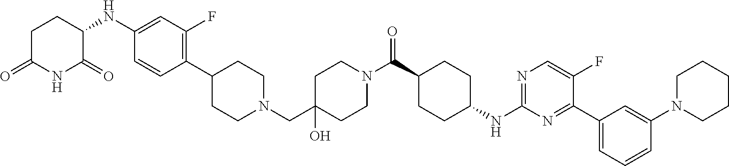 [SMILES: O=C1CC[C@H](Nc2ccc(C3CCN(CC4(O)CCN(C(=O)[C@H]5CC[C@H](Nc6ncc(F)c(-c7cccc(N8CCCCC8)c7)n6)CC5)CC4)CC3)c(F)c2)C(=O)N1] |
| | trans-3-((3-fluoro-4-(1-((1-(4-((5-fluoro-4-(3-(piperidin-1-yl)phenyl)pyrimidin-2-yl)amino)cyclohexane-1-carbonyl)- |
| | 4-hydroxypiperidin-4-yl)methyl)piperidin-4-yl)phenyl)amino)piperidine-2,6-dione |
| 515 | 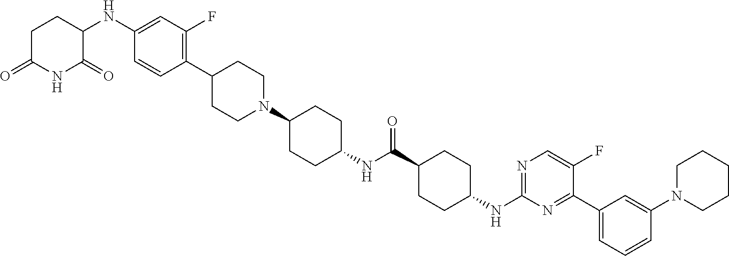 [SMILES: O=C1CCC(Nc2ccc(C3CCN([C@H]4CC[C@H](NC(=O)[C@H]5CC[C@H](Nc6ncc(F)c(-c7cccc(N8CCCCC8)c7)n6)CC5)CC4)CC3)c(F)c2)C(=O)N1] |
| | trans-N-(trans-4-(4-(4-((2,6-dioxopiperidin-3-yl)amino)-2-fluorophenyl)piperidin-1-yl)cyclohexyl)-4-((5-fluoro-4- |
| | (3-(piperidin-1-yl)phenyl)pyrimidin-2-yl)amino)cyclohexane-1-carboxamide |
| 516 | 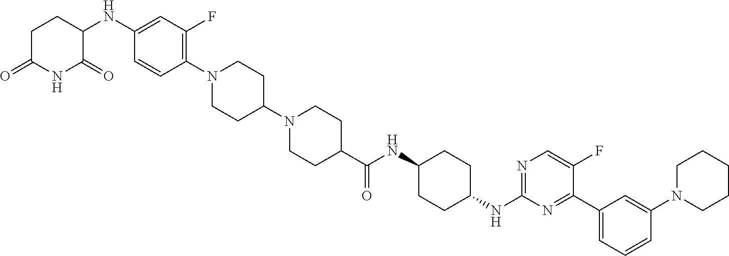 [SMILES: O=C1CCC(Nc2ccc(N3CCC(N4CCC(C(=O)N[C@H]5CC[C@H](Nc6ncc(F)c(-c7cccc(N8CCCCC8)c7)n6)CC5)CC4)CC3)c(F)c2)C(=O)N1] |
| | trans-1′-(4-((2,6-dioxopiperidin-3-yl)amino)-2-fluorophenyl)-N-(4-((5-fluoro-4-(3-(piperidin-1-yl)phenyl)pyrimidin- |
| | 2-yl)amino)cyclohexyl)-[1,4′-bipiperidine]-4-carboxamide |
| 517 | 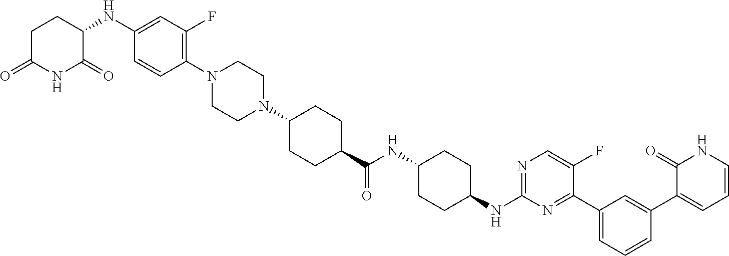 [SMILES: O=C1CC[C@H](Nc2ccc(N3CCN([C@H]4CC[C@H](C(=O)N[C@H]5CC[C@H](Nc6ncc(F)c(-c7cccc(-c8ccc[nH]c8=O)c7)n6)CC5)CC4)CC3)c(F)c2)C(=O)N1] |
| | trans-4-(4-(4-(((S)-2,6-dioxopiperidin-3-yl)amino)-2-fluorophenyl)piperazin-1-yl)-N-((trans)-4-((5-fluoro-4-(3-(2-oxo- |
| | 1,2-dihydropyridin-3-yl)phenyl)pyrimidin-2-yl)amino)cyclohexyl)cyclohexane-1-carboxamide |
| 518 | 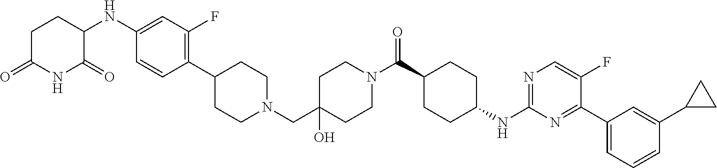 [SMILES: O=C1CCC(Nc2ccc(C3CCN(CC4(O)CCN(C(=O)[C@H]5CC[C@H](Nc6ncc(F)c(-c7cccc(C8CC8)c7)n6)CC5)CC4)CC3)c(F)c2)C(=O)N1] |
| | trans-3-((4-(1-((1-(4-((4-(3-cyclopropylphenyl)-5-fluoropyrimidin-2-yl)amino)cyclohexane-1-carbonyl)-4-hydroxy- |
| | piperidin-4-yl)methyl)piperidin-4-yl)-3-fluorophenyl)amino)piperidine-2,6-dione |
| 519 | 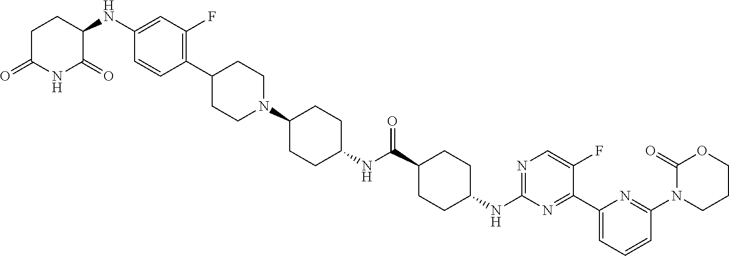 [SMILES: O=C1CC[C@@H](Nc2ccc(C3CCN([C@H]4CC[C@H](NC(=O)[C@H]5CC[C@H](Nc6ncc(F)c(-c7cccc(N8CCCOC8=O)n7)n6)CC5)CC4)CC3)c(F)c2)C(=O)N1] |
| | (trans)-N-((trans)-4-(4-(4-((2,6-dioxopiperidin-3-yl)amino)-2-fluorophenyl)piperidin-1-yl)cyclohexyl)-4-((5-fluoro-4- |
| | (6-(2-oxo-1,3-oxazinan-3-yl)pyridin-2-yl)pyrimidin-2-yl)amino)cyclohexane-1-carboxamide |
| 520 | 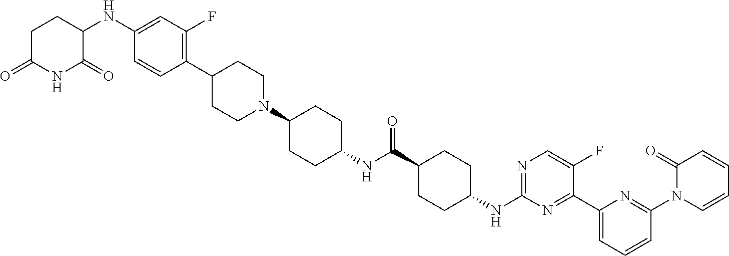 [SMILES: O=C1CCC(Nc2ccc(C3CCN([C@H]4CC[C@H](NC(=O)[C@H]5CC[C@H](Nc6ncc(F)c(-c7cccc(-n8ccccc8=O)n7)n6)CC5)CC4)CC3)c(F)c2)C(=O)N1] |
| | trans-N-(trans-4-(4-(4-((2,6-dioxopiperidin-3-yl)amino)-2-fluorophenyl)piperidin-1-yl)cyclohexyl)-4-((5-fluoro-4-(2- |
| | oxo-2H-[1,2′-bipyridin]-6′-yl)pyrimidin-2-yl)amino)cyclohexane-1-carboxamide |
| 521 | 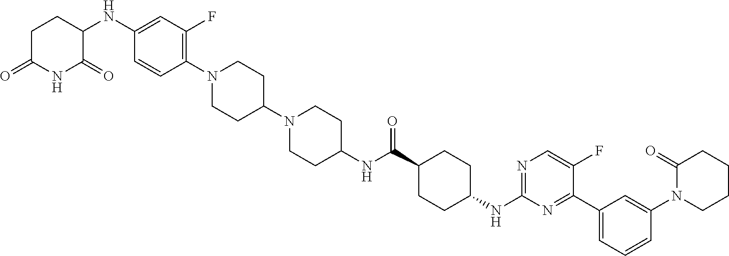 [SMILES: O=C1CCC(Nc2ccc(N3CCC(N4CCC(NC(=O)[C@H]5CC[C@H](Nc6ncc(F)c(-c7cccc(N8CCCCC8=O)c7)n6)CC5)CC4)CC3)c(F)c2)C(=O)N1] |
| | trans-N-(1′-(4-((2,6-dioxopiperidin-3-yl)amino)-2-fluorophenyl)-[1,4′-bipiperidin]-4-yl)-4-((5-fluoro-4-(3-(2-oxo- |
| | piperidin-1-yl)phenyl)pyrimidin-2-yl)amino)cyclohexane-1-carboxamide |
| 522 | 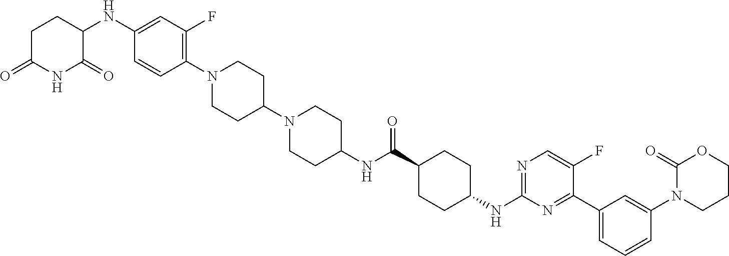 [SMILES: O=C1CCC(Nc2ccc(N3CCC(N4CCC(NC(=O)[C@H]5CC[C@H](Nc6ncc(F)c(-c7cccc(N8CCCOC8=O)c7)n6)CC5)CC4)CC3)c(F)c2)C(=O)N1] |
| | trans-N-(1′-(4-((2,6-dioxopiperidin-3-yl)amino)-2-fluorophenyl)-[1,4′-bipiperidin]-4-yl)-4-((5-fluoro-4-(3-(2-oxo-1,3- |
| | oxazinan-3-yl)phenyl)pyrimidin-2-yl)amino)cyclohexane-1-carboxamide |
| 523 | 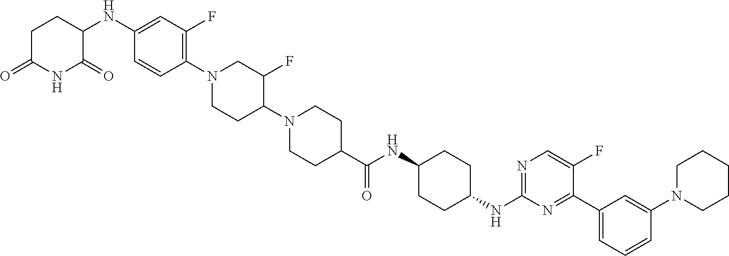 [SMILES: O=C1CCC(Nc2ccc(N3CCC(N4CCC(C(=O)N[C@H]5CC[C@H](Nc6ncc(F)c(-c7cccc(N8CCCCC8)c7)n6)CC5)CC4)C(F)C3)c(F)c2)C(=O)N1] |
| | 1′-(4-((2,6-dioxopiperidin-3-yl)amino)-2-fluorophenyl)-3′-fluoro-N-((1r,4r)-4-((5-fluoro-4-(3-(piperidin-1-yl)phenyl) |
| | pyrimidin-2-yl)amino)cyclohexyl)-[1,4′-bipiperidine]-4-carboxamide |
| 524 | 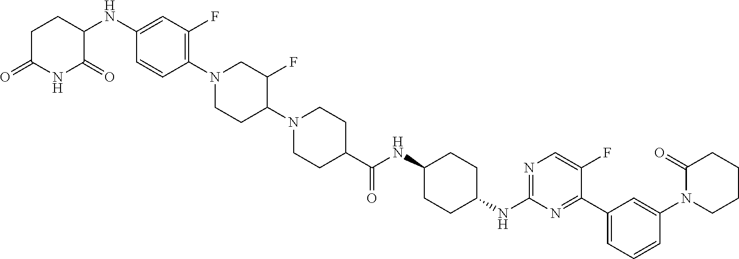 [SMILES: O=C1CCC(Nc2ccc(N3CCC(N4CCC(C(=O)N[C@H]5CC[C@H](Nc6ncc(F)c(-c7cccc(N8CCCCC8=O)c7)n6)CC5)CC4)C(F)C3)c(F)c2)C(=O)N1] |
| | 1′-(4-((2,6-dioxopiperidin-3-yl)amino)-2-fluorophenyl)-3′-fluoro-N-((1r,4r)-4-((5-fluoro-4-(3-(2-oxopiperidin-1-yl)- |
| | phenyl)pyrimidin-2-yl)amino)cyclohexyl)-[1,4′-bipiperidine]-4-carboxamide |
| 525 | 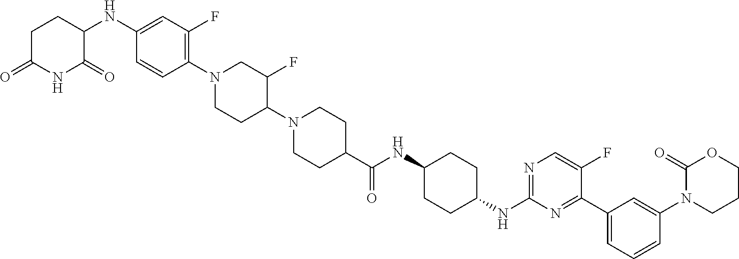 [SMILES: O=C1CCC(Nc2ccc(N3CCC(N4CCC(C(=O)N[C@H]5CC[C@H](Nc6ncc(F)c(-c7cccc(N8CCCOC8=O)c7)n6)CC5)CC4)C(F)C3)c(F)c2)C(=O)N1] |
| | 1′-(4-((2,6-dioxopiperidin-3-yl)amino)-2-fluorophenyl)-3′-fluoro-N-((1r,4r)-4-((5-fluoro-4-(3-(2-oxo-1,3-oxazinan-3- |
| | yl)phenyl)pyrimidin-2-yl)amino)cyclohexyl)-[1,4′-bipiperidine]-4-carboxamide |
| 526 | 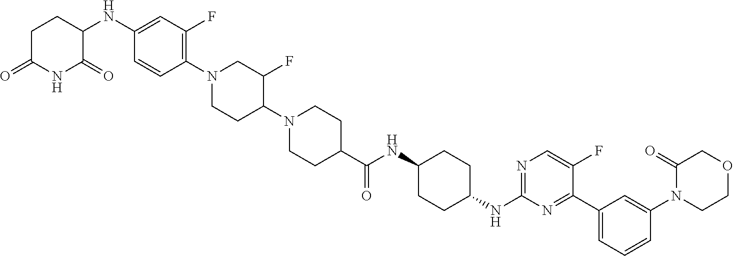 [SMILES: O=C1CCC(Nc2ccc(N3CCC(N4CCC(C(=O)N[C@H]5CC[C@H](Nc6ncc(F)c(-c7cccc(N8CCOCC8=O)c7)n6)CC5)CC4)C(F)C3)c(F)c2)C(=O)N1] |
| | 1′-(4-((2,6-dioxopiperidin-3-yl)amino)-2-fluorophenyl)-3′-fluoro-N-((1r,4r)-4-((5-fluoro-4-(3-(3-oxomorpholino)phen- |
| | yl)pyrimidin-2-yl)amino)cyclohexyl)-[1,4′-bipiperidine]-4-carboxamide |
| 527 | 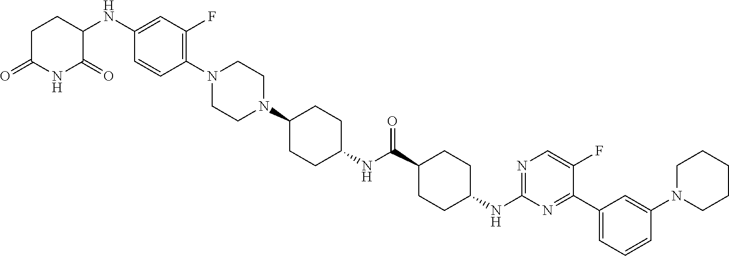 [SMILES: O=C1CCC(Nc2ccc(N3CCN([C@H]4CC[C@H](NC(=O)[C@H]5CC[C@H](Nc6ncc(F)c(-c7cccc(N8CCCCC8)c7)n6)CC5)CC4)CC3)c(F)c2)C(=O)N1] |
| | trans-(1r,4R)-N-((1r,4R)-4-(4-(4-((2,6-dioxopiperidin-3-yl)amino)-2-fluorophenyl)piperazin-1-yl)cyclohexyl)-4- |
| | ((5-fluoro-4-(3-(piperidin-1-yl)phenyl)pyrimidin-2-yl)amino)cyclohexane-1-carboxamide |
| 528 | 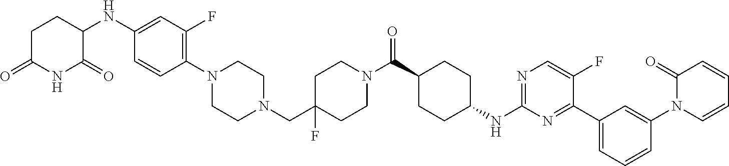 [SMILES: O=C1CCC(Nc2ccc(N3CCN(CC4(F)CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-3-((3-fluoro-4-(4-((4-fluoro-1-(4-((5-fluoro-4-(3-(2-oxopyridin-1(2H)-yl)phenyl)pyrimidin-2-yl)amino)cyclo- |
| | hexane-1-carbonyl)piperidin-4-yl)methyl)piperazin-1-yl)phenyl)amino)piperidine-2,6-dione |
| 529 | 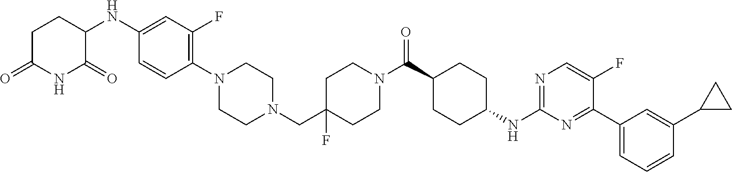 [SMILES: O=C1CCC(Nc2ccc(N3CCN(CC4(F)CCN(C(=O)[C@H]5CC[C@H](Nc6ncc(F)c(-c7cccc(C8CC8)c7)n6)CC5)CC4)CC3)c(F)c2)C(=O)N1] |
| | trans-3-((4-(4-((1-((1r,4r)-4-((4-(3-cyclopropylphenyl)-5-fluoropyrimidin-2-yl)amino)cyclohexane-1-carbonyl)-4-fluoro- |
| | piperidin-4-yl)methyl)piperazin-1-yl)-3-fluorophenyl)amino)piperidine-2,6-dione |
| 530 | 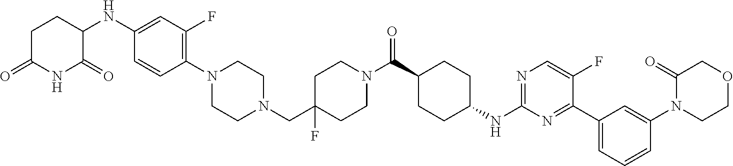 [SMILES: O=C1CCC(Nc2ccc(N3CCN(CC4(F)CCN(C(=O)[C@H]5CC[C@H](Nc6ncc(F)c(-c7cccc(N8CCOCC8=O)c7)n6)CC5)CC4)CC3)c(F)c2)C(=O)N1] |
| | trans-3-((3-fluoro-4-(4-((4-fluoro-1-(4-((5-fluoro-4-(3-(3-oxomorpholino)phenyl)pyrimidin-2-yl)amino)cyclohexane- |
| | 1-carbonyl)piperidin-4-yl)methyl)piperazin-1-yl)phenyl)amino)piperidine-2,6-dione |
| 531 | 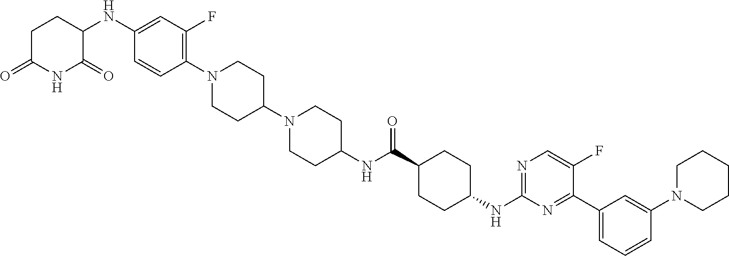 [SMILES: O=C1CCC(Nc2ccc(N3CCC(N4CCC(NC(=O)[C@H]5CC[C@H](Nc6ncc(F)c(-c7cccc(N8CCCCC8)c7)n6)CC5)CC4)CC3)c(F)c2)C(=O)N1] |
| | trans-N-(1′-(4-((2,6-dioxopiperidin-3-yl)amino)-2-fluorophenyl)-[1,4′-bipiperidin]-4-yl)-4-((5-fluoro-4-(3-(piperidin- |
| | 1-yl)phenyl)pyrimidin-2-yl)amino)cyclohexane-1-carboxamide |
| 532 | 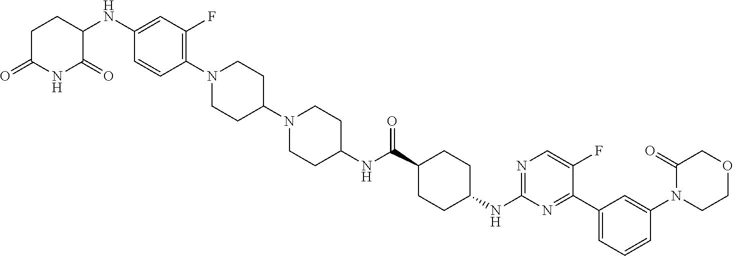 [SMILES: O=C1CCC(Nc2ccc(N3CCC(N4CCC(NC(=O)[C@H]5CC[C@H](Nc6ncc(F)c(-c7cccc(N8CCOCC8=O)c7)n6)CC5)CC4)CC3)c(F)c2)C(=O)N1] |
| | trans-N-(1′-(4-((2,6-dioxopiperidin-3-yl)amino)-2-fluorophenyl)-[1,4′-bipiperidin]-4-yl)-4-((5-fluoro-4-(3-(3-oxo- |
| | morpholino)phenyl)pyrimidin-2-yl)amino)cyclohexane-1-carboxamide |
| 533 | 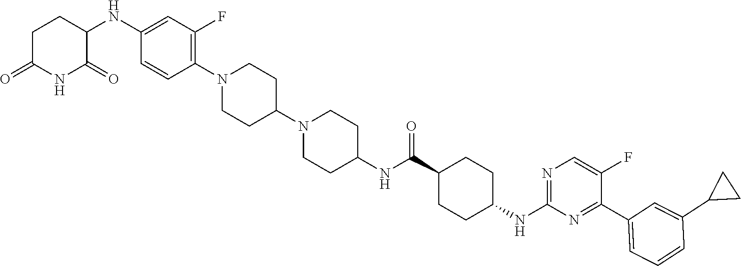 [SMILES: O=C1CCC(Nc2ccc(N3CCC(N4CCC(NC(=O)[C@H]5CC[C@H](Nc6ncc(F)c(-c7cccc(C8CC8)c7)n6)CC5)CC4)CC3)c(F)c2)C(=O)N1] |
| | trans-4-((4-(3-cyclopropylphenyl)-5-fluoropyrimidin-2-yl)amino)-N-(1′-(4-((2,6-dioxopiperidin-3-yl)amino)-2-fluoro- |
| | phenyl)-[1,4′-bipiperidin]-4-yl)cyclohexane-1-carboxamide |
| 534 | 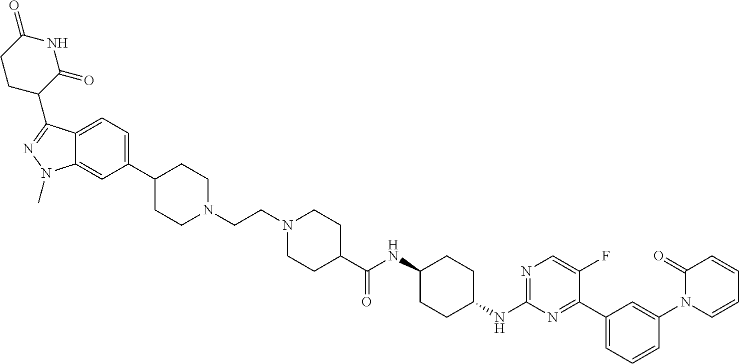 [SMILES: Cn1nc(C2CCC(=O)NC2=O)c2ccc(C3CCN(CCN4CCC(C(=O)N[C@H]5CC[C@H](Nc6ncc(F)c(-c7cccc(-n8ccccc8=O)c7)n6)CC5)CC4)CC3)cc21] |
| | 1-(2-(4-(3-(2,6-dioxopiperidin-3-yl)-1-methyl-1H-indazol-6-yl)piperidin-1-yl)ethyl)-N-((1r,4r)-4-((5-fluoro-4-(3-(2- |
| | oxopyridin-1(2H)-yl)phenyl)pyrimidin-2-yl)amino)cyclohexyl)piperidine-4-carboxamide |
| 535 | 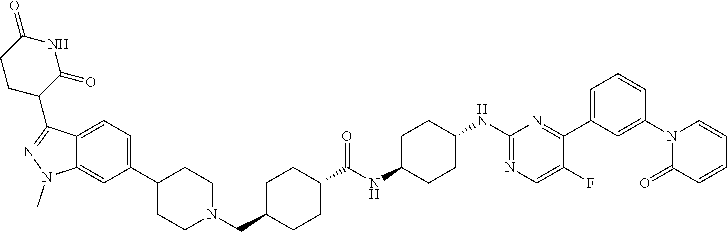 [SMILES: Cn1nc(C2CCC(=O)NC2=O)c2ccc(C3CCN(C[C@H]4CC[C@H](C(=O)N[C@H]5CC[C@H](Nc6ncc(F)c(-c7cccc(-n8ccccc8=O)c7)n6)CC5)CC4)CC3)cc21] |
| | trans-(1r,4R)-4-((4-(3-(2,6-dioxopiperidin-3-yl)-1-methyl-1H-indazol-6-yl)piperidin-1-yl)methyl)-N-((1r,4R)-4-((5- |
| | fluoro-4-(3-(2-oxopyridin-1(2H)-yl)phenyl)pyrimidin-2-yl)amino)cyclohexyl)cyclohexane-1-carboxamide |
| 536 | 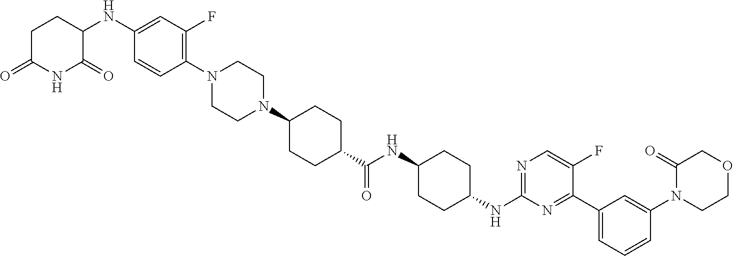 [SMILES: O=C1CCC(Nc2ccc(N3CCN([C@H]4CC[C@H](C(=O)N[C@H]5CC[C@H](Nc6ncc(F)c(-c7cccc(N8CCOCC8=O)c7)n6)CC5)CC4)CC3)c(F)c2)C(=O)N1] |
| | trans-4-(4-(4-((2,6-dioxopiperidin-3-yl)amino)-2-fluorophenyl)piperazin-1-yl)-N-(trans-4-((5-fluoro-4-(3-(3-oxo- |
| | morpholino)phenyl)pyrimidin-2-yl)amino)cyclohexyl)cyclohexane-1-carboxamide |
| 537 | 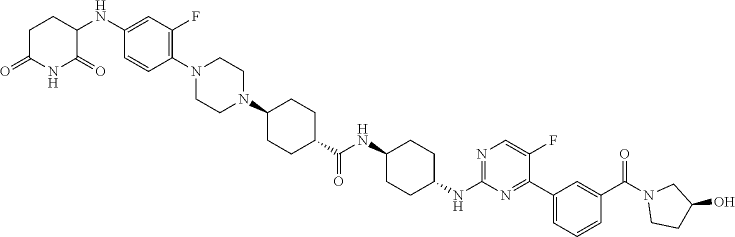 [SMILES: O=C1CCC(Nc2ccc(N3CCN([C@H]4CC[C@H](C(=O)N[C@H]5CC[C@H](Nc6ncc(F)c(-c7cccc(C(=O)N8CC[C@H](O)C8)c7)n6)CC5)CC4)CC3)c(F)c2)C(=O)N1] |
| | trans-4-(4-(4-((2,6-dioxopiperidin-3-yl)amino)-2-fluorophenyl)piperazin-1-yl)-N-(trans-4-((5-fluoro-4-(3-((S)-3-hydroxy- |
| | pyrrolidine-1-carbonyl)phenyl)pyrimidin-2-yl)amino)cyclohexyl)cyclohexane-1-carboxamide |
| 538 | 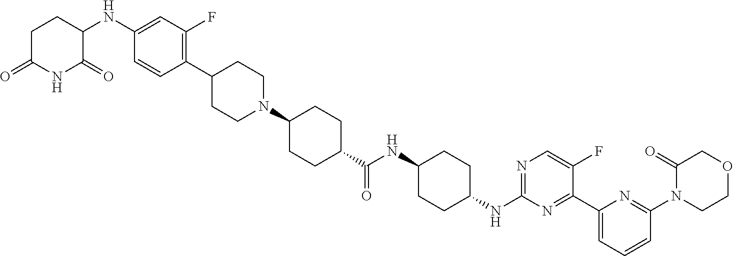 [SMILES: O=C1CCC(Nc2ccc(C3CCN([C@H]4CC[C@H](C(=O)N[C@H]5CC[C@H](Nc6ncc(F)c(-c7cccc(N8CCOCC8=O)n7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4-(6-(3- |
| | oxomorpholino)pyridin-2-yl)pyrimidin-2-yl)amino)cyclohexyl)cyclohexane-1-carboxamide |
| 539 | 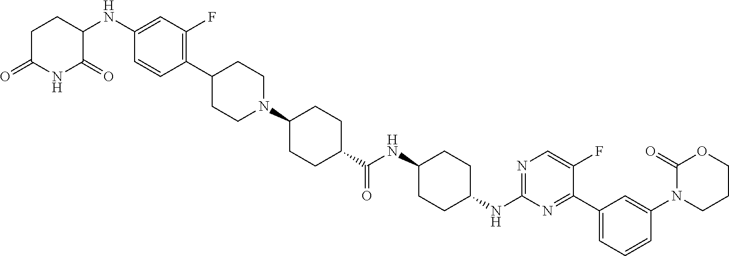 [SMILES: O=C1CCC(Nc2ccc(C3CCN([C@H]4CC[C@H](C(=O)N[C@H]5CC[C@H](Nc6ncc(F)c(-c7cccc(N8CCCOC8=O)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4- |
| | (6-(2-oxo-1,3-oxazinan-3-yl)pyridin-2-yl)pyrimidin-2-yl)amino)cyclohexyl)cyclohexane-1-carboxamide |
| 540 | 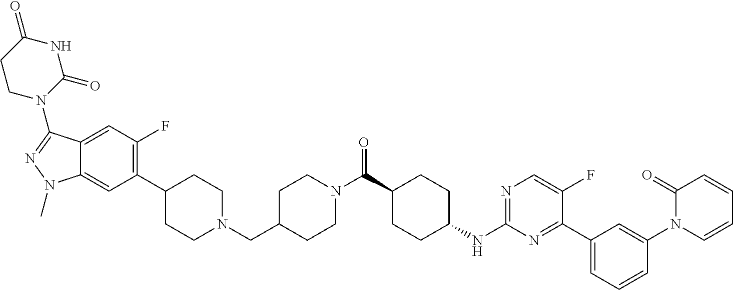 [SMILES: Cn1nc(N2CCC(=O)NC2=O)c2cc(F)c(C3CCN(CC4CCN(C(=O)[C@H]5CC[C@H](Nc6ncc(F)c(-c7cccc(-n8ccccc8=O)c7)n6)CC5)CC4)CC3)cc21] |
| | trans-1-((1-(4-((2,6-dioxopiperidin-3-yl)amino)-2-fluorophenyl)piperidin-4-yl)methyl)-N-((1r,4r)-4-((5-fluoro-4-(3-(2- |
| | oxopyridin-1(2H)-yl)phenyl)pyrimidin-2-yl)amino)cyclohexyl)piperidine-4-carboxamide |
| 541 | 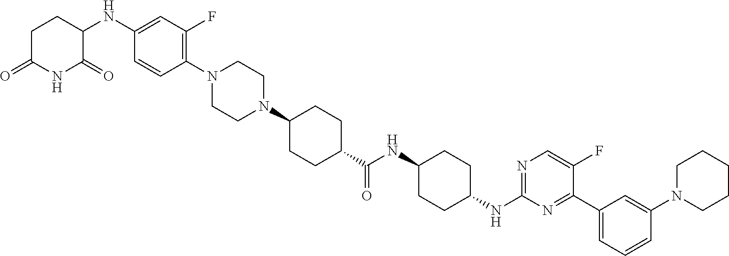 [SMILES: O=C1CCC(Nc2ccc(N3CCN([C@H]4CC[C@H](C(=O)N[C@H]5CC[C@H](Nc6ncc(F)c(-c7cccc(N8CCCCC8)c7)n6)CC5)CC4)CC3)c(F)c2)C(=O)N1] |
| | trans-4-(4-(4-((2,6-dioxopiperidin-3-yl)amino)-2-fluorophenyl)piperazin-1-yl)-N-(trans-4-((5-fluoro-4-(3-(piperidin-1- |
| | yl)phenyl)pyrimidin-2-yl)amino)cyclohexyl)cyclohexane-1-carboxamide |
| 542 | 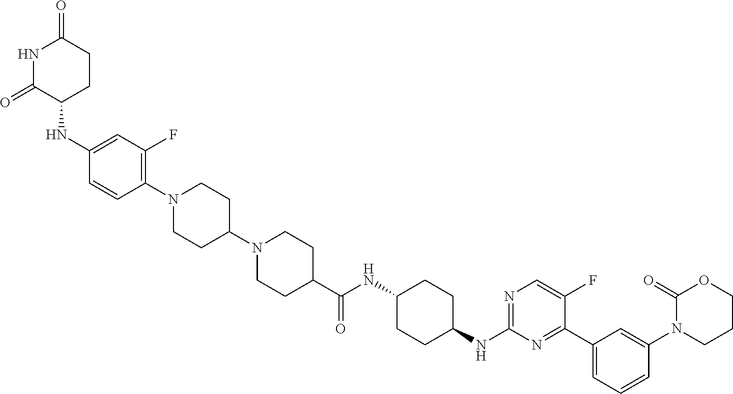 [SMILES: O=C1CC[C@H](Nc2ccc(N3CCC(N4CCC(C(=O)N[C@H]5CC[C@H](Nc6ncc(F)c(-c7cccc(N8CCCOC8=O)c7)n6)CC5)CC4)CC3)c(F)c2)C(=O)N1] |
| | 1′-(4-(((S)-2,6-dioxopiperidin-3-yl)amino)-2-fluorophenyl)-N-((trans)-4-((5-fluoro-4-(3-(2-oxo-1,3-oxazinan-3-yl)- |
| | phenyl)pyrimidin-2-yl)amino)cyclohexyl)-[1,4′-bipiperidine]-4-carboxamide |
| 543 | 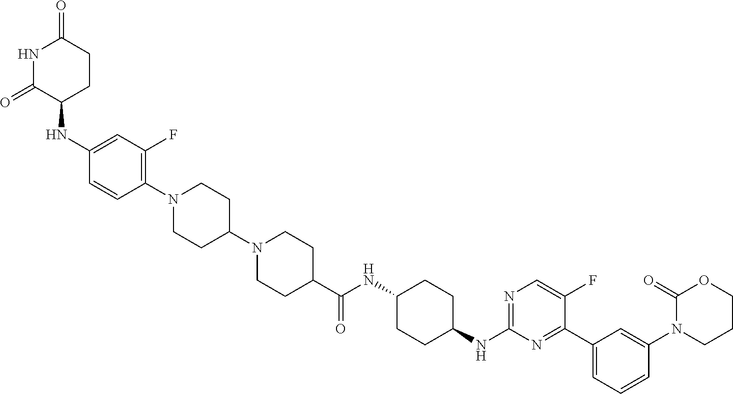 [SMILES: O=C1CC[C@@H](Nc2ccc(N3CCC(N4CCC(C(=O)N[C@H]5CC[C@H](Nc6ncc(F)c(-c7cccc(N8CCCOC8=O)c7)n6)CC5)CC4)CC3)c(F)c2)C(=O)N1] |
| | 1′-(4-(((R)-2,6-dioxopiperidin-3-yl)amino)-2-fluorophenyl)-N-((trans)-4-((5-fluoro-4-(3-(2-oxo-1,3-oxazinan-3-yl)- |
| | phenyl)pyrimidin-2-yl)amino)cyclohexyl)-[1,4′-bipiperidine]-4-carboxamide |
| 544 | 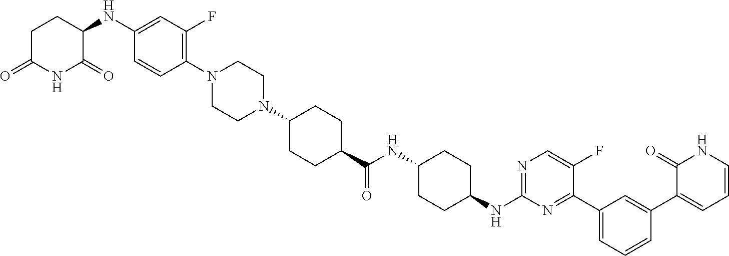 [SMILES: O=C1CC[C@@H](Nc2ccc(N3CCN([C@H]4CC[C@H](C(=O)N[C@H]5CC[C@H](Nc6ncc(F)c(-c7cccc(-c8ccc[nH]c8=O)c7)n6)CC5)CC4)CC3)c(F)c2)C(=O)N1] |
| | trans-4-(4-(4-(((R)-2,6-dioxopiperidin-3-yl)amino)-2-fluorophenyl)piperazin-1-yl)-N-((trans)-4-((5-fluoro-4-(3-(2-oxo- |
| | 1,2-dihydropyridin-3-yl)phenyl)pyrimidin-2-yl)amino)cyclohexyl)cyclohexane-1-carboxamide |
| 545 | 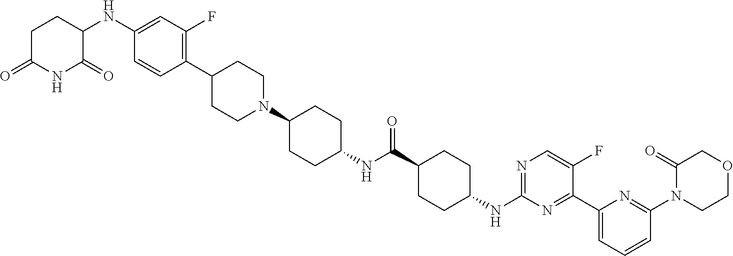 [SMILES: O=C1CCC(Nc2ccc(C3CCN([C@H]4CC[C@H](NC(=O)[C@H]5CC[C@H](Nc6ncc(F)c(-c7cccc(N8CCOCC8=O)n7)n6)CC5)CC4)CC3)c(F)c2)C(=O)N1] |
| | trans-N-(trans-4-(4-(4-((2,6-dioxopiperidin-3-yl)amino)-2-fluorophenyl)piperidin-1-yl)cyclohexyl)-4-((5-fluoro-4-(6-(3- |
| | oxomorpholino)pyridin-2-yl)pyrimidin-2-yl)amino)cyclohexane-1-carboxamide |
| 546 | 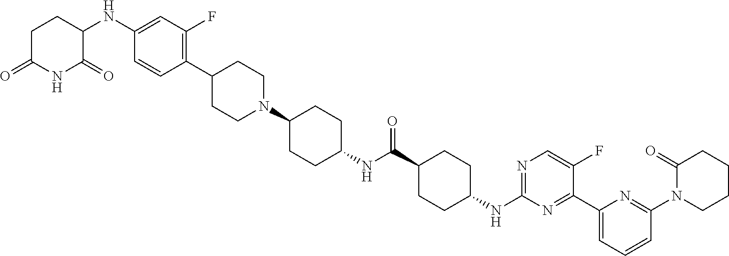 [SMILES: O=C1CCC(Nc2ccc(C3CCN([C@H]4CC[C@H](NC(=O)[C@H]5CC[C@H](Nc6ncc(F)c(-c7cccc(N8CCCCC8=O)n7)n6)CC5)CC4)CC3)c(F)c2)C(=O)N1] |
| | trans-N-(trans-4-(4-(4-((2,6-dioxopiperidin-3-yl)amino)-2-fluorophenyl)piperidin-1-yl)cyclohexyl)-4-((5-fluoro-4-(6- |
| | (2-oxopiperidin-1-yl)pyridin-2 --- )pyrimidin-2-yl)amino)cyclohexane-1-carboxamide |
| 547 | 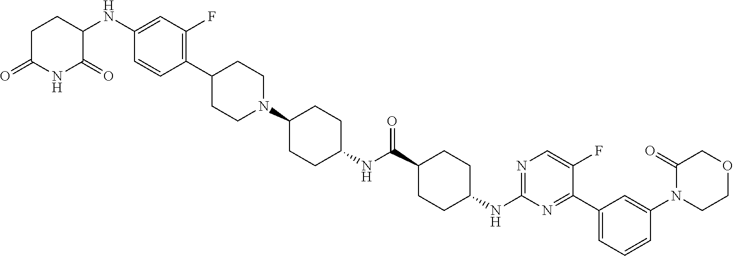 [SMILES: O=C1CCC(Nc2ccc(C3CCN([C@H]4CC[C@H](NC(=O)[C@H]5CC[C@H](Nc6ncc(F)c(-c7cccc(N8CCOCC8=O)c7)n6)CC5)CC4)CC3)c(F)c2)C(=O)N1] |
| | trans-N-(trans-4-(4-(4-((2,6-dioxopiperidin-3-yl)amino)-2-fluorophenyl)piperidin-1-yl)cyclohexyl)-4-((5-fluoro-4-(3- |
| | (3-oxomorpholino)phenyl)pyrimidin-2-yl)amino)cyclohexane-1-carboxamide |
| 548 | 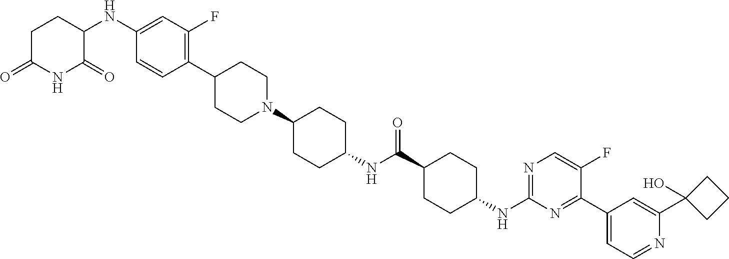 [SMILES: O=C1CCC(Nc2ccc(C3CCN([C@H]4CC[C@H](NC(=O)[C@H]5CC[C@H](Nc6ncc(F)c(-c7ccnc(C8(O)CCC8)c7)n6)CC5)CC4)CC3)c(F)c2)C(=O)N1] |
| | trans-N-(trans-4-(4-(4-((2,6-dioxopiperidin-3-yl)amino)-2-fluorophenyl)piperidin-1-yl)cyclohexyl)-4-((5-fluoro-4- |
| | (2-(1-hydroxycyclobutyl)pyridin-4-yl)pyrimidin-2-yl)amino)cyclohexane-1-carboxamide |
| 549 | 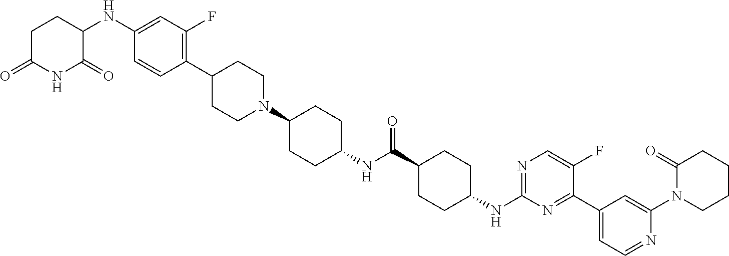 [SMILES: O=C1CCC(Nc2ccc(C3CCN([C@H]4CC[C@H](NC(=O)[C@H]5CC[C@H](Nc6ncc(F)c(-c7ccnc(N8CCCCC8=O)c7)n6)CC5)CC4)CC3)c(F)c2)C(=O)N1] |
| | trans-N-(trans-4-(4-(4-((2,6-dioxopiperidin-3-yl)amino)-2-fluorophenyl)piperidin-1-yl)cyclohexyl)-4-((5-fluoro-4-(2- |
| | (2-oxopiperidin-1-yl)pyridin-4-yl)pyrimidin-2-yl)amino)cyclohexane-1-carboxamide |
| 550 | 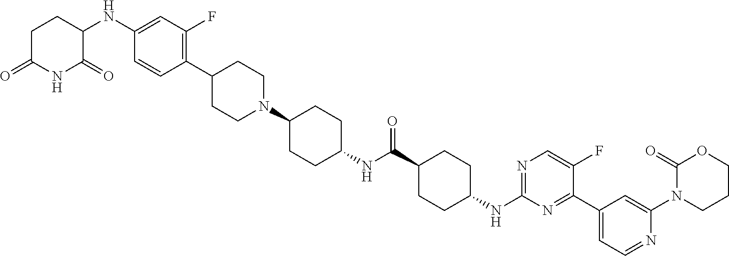 [SMILES: O=C1CCC(Nc2ccc(C3CCN([C@H]4CC[C@H](NC(=O)[C@H]5CC[C@H](Nc6ncc(F)c(-c7ccnc(N8CCCOC8=O)c7)n6)CC5)CC4)CC3)c(F)c2)C(=O)N1] |
| | trans-N-((trans)-4-(4-(4-((2,6-dioxopiperidin-3-yl)amino)-2-fluorophenyl)piperidin-1-yl)cyclohexyl)-4-((5-fluoro-4- |
| | (2-(2-oxo-1,3-oxazinan-3-yl)pyridin-4-yl)pyrimidin-2-yl)amino)cyclohexane-1-carboxamide |
| 551 | 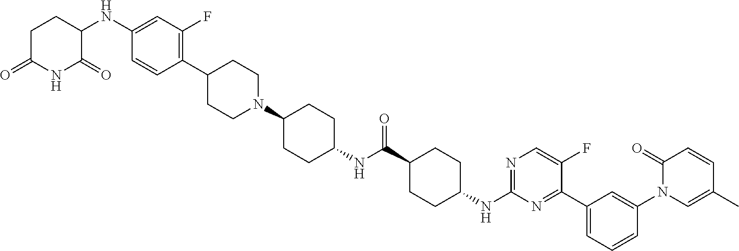 [SMILES: Cc1ccc(=O)n(-c2cccc(-c3nc(N[C@H]4CC[C@H](C(=O)N[C@H]5CC[C@H](N6CCC(c7ccc(NC8CCC(=O)NC8=O)cc7F)CC6)CC5)CC4)ncc3F)c2)c1] |
| | trans-N-(trans-4-(4-(4-((2,6-dioxopiperidin-3-yl)amino)-2-fluorophenyl)piperidin-1-yl)cyclohexyl)-4-((5-fluoro-4-(3- |
| | (5-methyl-2-oxopyridin-1(2H)-yl)phenyl)pyrimidin-2-yl)amino)cyclohexane-1-carboxamide |
| 552 | 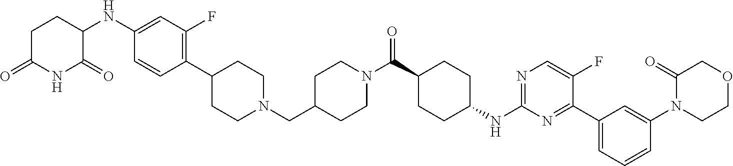 [SMILES: O=C1CCC(Nc2ccc(C3CCN(CC4CCN(C(=O)[C@H]5CC[C@H](Nc6ncc(F)c(-c7cccc(N8CCOCC8=O)c7)n6)CC5)CC4)CC3)c(F)c2)C(=O)N1] |
| | trans-3-((3-fluoro-4-(1-((1-(4-((5-fluoro-4-(3-(3-oxomorpholino)phenyl)pyrimidin-2-yl)amino)cyclohexane-1- |
| | carbonyl)piperidin-4-yl)methyl)piperidin-4-yl)phenyl)amino)piperidine-2,6-dione |
| 553 | 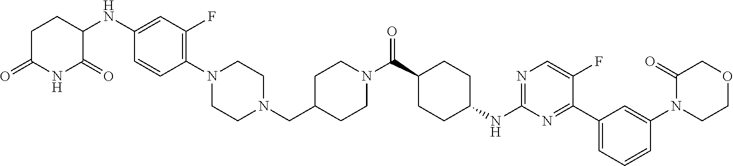 [SMILES: O=C1CCC(Nc2ccc(N3CCN(CC4CCN(C(=O)[C@H]5CC[C@H](Nc6ncc(F)c(-c7cccc(N8CCOCC8=O)c7)n6)CC5)CC4)CC3)c(F)c2)C(=O)N1] |
| | trans-3-((3-fluoro-4-(4-((1-(4-((5-fluoro-4-(3-(3-oxomorpholino)phenyl)pyrimidin-2-yl)amino)cyclohexane-1- |
| | carbonyl)piperidin-4-yl)methyl)piperazin-1-yl)phenyl)amino)piperidine-2,6-dione |
| 554 | 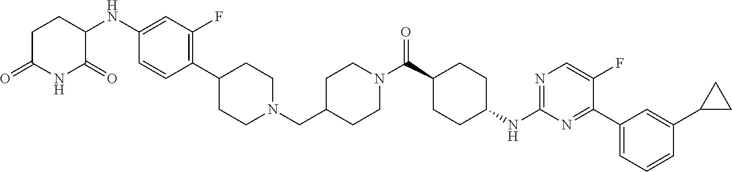 [SMILES: O=C1CCC(Nc2ccc(C3CCN(CC4CCN(C(=O)[C@H]5CC[C@H](Nc6ncc(F)c(-c7cccc(C8CC8)c7)n6)CC5)CC4)CC3)c(F)c2)C(=O)N1] |
| | trans-3-((4-(1-((1-(4-((4-(3-cyclopropylphenyl)-5-fluoropyrimidin-2-yl)amino)cyclohexane-1-carbonyl)piperidin-4-yl)- |
| | methyl)piperidin-4-yl)-3-fluorophenyl)amino)piperidine-2,6-dione |
| 555 | 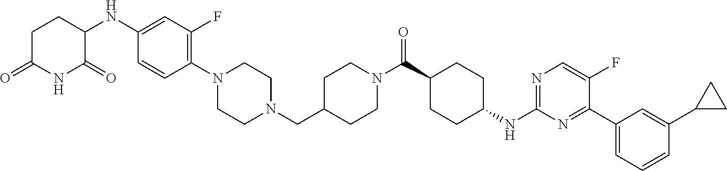 [SMILES: O=C1CCC(Nc2ccc(N3CCN(CC4CCN(C(=O)[C@H]5CC[C@H](Nc6ncc(F)c(-c7cccc(C8CC8)c7)n6)CC5)CC4)CC3)c(F)c2)C(=O)N1] |
| | trans-3-[4-[4-[[1-[4-[4-(3-cyclopropylphenyl)-5-fluoro-pyrimidin-2-yl]amino]cyclohexanecarbonyl]-4-piperidyl]methyl] |
| | piperazin-1-yl]-3-fluoro-anilino]piperidine-2,6-dione |
| 556 | 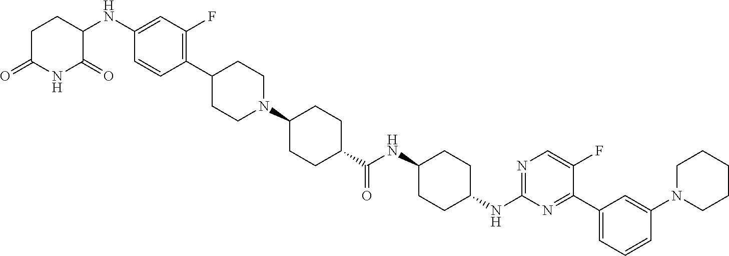 [SMILES: O=C1CCC(Nc2ccc(C3CCN([C@H]4CC[C@H](C(=O)N[C@H]5CC[C@H](Nc6ncc(F)c(-c7cccc(N8CCCCC8)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4- |
| | (3-(piperidin-1-yl)phenyl)pyrimidin-2-yl)amino)cyclohexyl)cyclohexane-1-carboxamide |
| 557 | 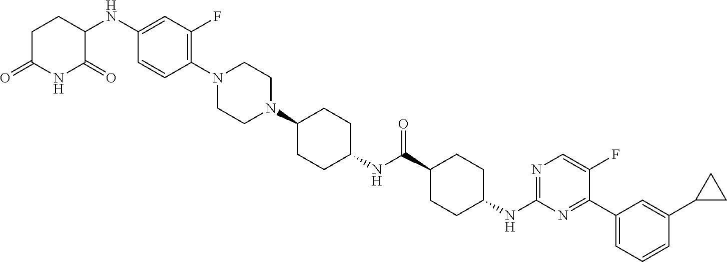 [SMILES: O=C1CCC(Nc2ccc(N3CCN([C@H]4CC[C@H](NC(=O)[C@H]5CC[C@H](Nc6ncc(F)c(-c7cccc(C8CC8)c7)n6)CC5)CC4)CC3)c(F)c2)C(=O)N1] |
| | trans-(1r,4R)-4-((4-(3-cyclopropylphenyl)-5-fluoropyrimidin-2-yl)amino)-N-((1r,4R)-4-(4-(4-((2,6-dioxopiperidin- |
| | 3-yl)amino)-2-fluorophenyl)piperazin-1-yl)cyclohexyl)cyclohexane-1-carboxamide |
| 558 | 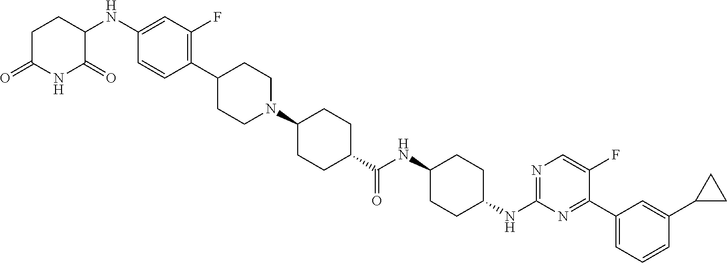 [SMILES: O=C1CCC(Nc2ccc(C3CCN([C@H]4CC[C@H](C(=O)N[C@H]5CC[C@H](Nc6ncc(F)c(-c7cccc(C8CC8)c7)n6)CC5)CC4)CC3)c(F)c2)C(=O)N1] |
| | trans-(1r,4R)-N-((1r,4R)-4-((4-(3-cyclopropylphenyl)-5-fluoropyrimidin-2-yl)amino)cyclohexyl)-4-(4-(4-((2,6- |
| | dioxopiperidin-3-yl)amino)-2-fluorophenyl)piperidin-1-yl)cyclohexane-1-carboxamide |
| 559 | 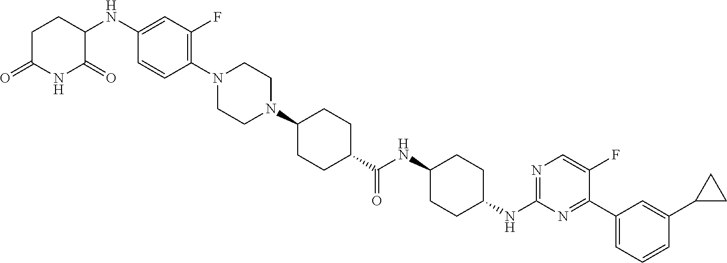 [SMILES: O=C1CCC(Nc2ccc(N3CCN([C@H]4CC[C@H](C(=O)N[C@H]5CC[C@H](Nc6ncc(F)c(-c7cccc(C8CC8)c7)n6)CC5)CC4)CC3)c(F)c2)C(=O)N1] |
| | trans-N-(trans-4-((4-(3-cyclopropylphenyl)-5-fluoropyrimidin-2-yl)amino)cyclohexyl)-4-(4-(4-((2,6-dioxopiperidin- |
| | 3-yl)amino)-2-fluorophenyl)piperazin-1-yl)cyclohexane-1-carboxamide |
| 560 | 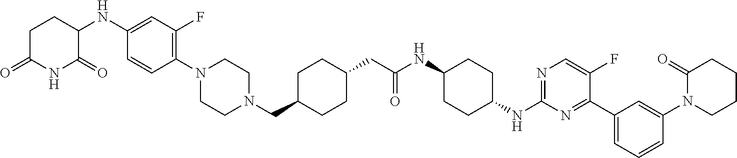 [SMILES: O=C1CCC(Nc2ccc(N3CCN(C[C@H]4CC[C@H](CC(=O)N[C@H]5CC[C@H](Nc6ncc(F)c(-c7cccc(N8CCCCC8=O)c7)n6)CC5)CC4)CC3)c(F)c2)C(=O)N1] |
| | trans-2-((1r,4R)-4-((4-(4-((2,6-dioxopiperidin-3-yl)amino)-2-fluorophenyl)piperazin-1-yl)methyl)cyclohexyl)-N- |
| | ((1r,4R)-4-((5-fluoro-4-(3-(2-oxopiperidin-1-yl)phenyl)pyrimidin-2-yl)amino)cyclohexyl)acetamide |
| 561 | 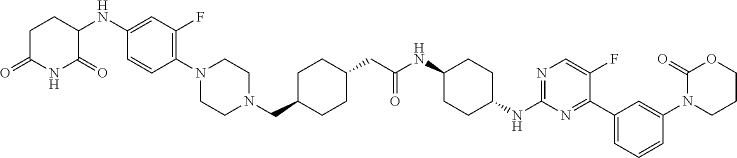 [SMILES: O=C1CCC(Nc2ccc(N3CCN(C[C@H]4CC[C@H](CC(=O)N[C@H]5CC[C@H](Nc6ncc(F)c(-c7cccc(N8CCCOC8=O)c7)n6)CC5)CC4)CC3)c(F)c2)C(=O)N1] |
| | trans-2-((1r,4R)-4-((4-(4-((2,6-dioxopiperidin-3-yl)amino)-2-fluorophenyl)piperazin-1-yl)methyl)cyclohexyl)-N- |
| | ((1r,4R)-4-((5-fluoro-4-(3-(2-oxo-1,3-oxazinan-3-yl)phenyl)pyrimidin-2-yl)amino)cyclohexyl)acetamide |
| 562 | 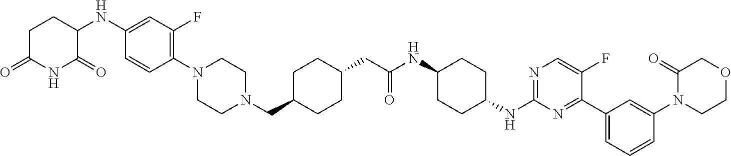 [SMILES: O=C1CCC(Nc2ccc(N3CCN(C[C@H]4CC[C@H](CC(=O)N[C@H]5CC[C@H](Nc6ncc(F)c(-c7cccc(N8CCOCC8=O)c7)n6)CC5)CC4)CC3)c(F)c2)C(=O)N1] |
| | trans-2-((1r,4R)-4-((4-(4-((2,6-dioxopiperidin-3-yl)amino)-2-fluorophenyl)piperazin-1-yl)methyl)cyclohexyl)-N- |
| | ((1r,4R)-4-((5-fluoro-4-(3-(3-oxomorpholino)phenyl)pyrimidin-2-yl)amino)cyclohexyl)acetamide |
| 563 | 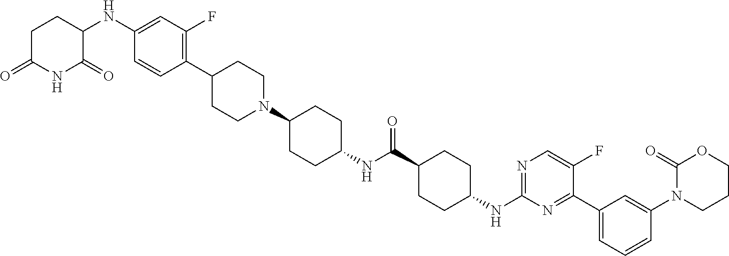 [SMILES: O=C1CCC(Nc2ccc(C3CCN([C@H]4CC[C@H](NC(=O)[C@H]5CC[C@H](Nc6ncc(F)c(-c7cccc(N8CCCOC8=O)c7)n6)CC5)CC4)CC3)c(F)c2)C(=O)N1] |
| | trans-N-(trans-4-(4-(4-((2,6-dioxopiperidin-3-yl)amino)-2-fluorophenyl)piperidin-1-yl)cyclohexyl)-4-((5-fluoro- |
| | 4-(3-(2-oxo-1,3-oxazinan-3-yl)phenyl)pyrimidin-2-yl)amino)cyclohexane-1-carboxamide |
| 564 | 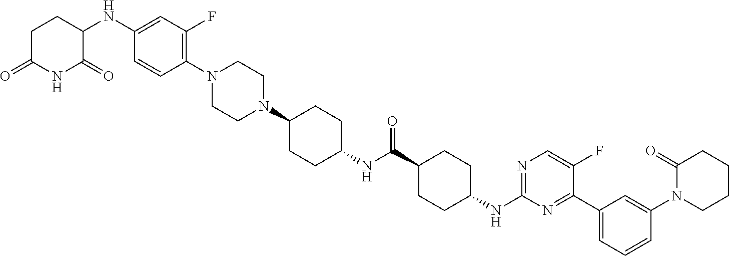 [SMILES: O=C1CCC(Nc2ccc(N3CCN([C@H]4CC[C@H](NC(=O)[C@H]5CC[C@H](Nc6ncc(F)c(-c7cccc(N8CCCCC8=O)c7)n6)CC5)CC4)CC3)c(F)c2)C(=O)N1] |
| | (1r,4R)-N-((1r,4R)-4-(4-(4-((2,6-dioxopiperidin-3-yl)amino)-2-fluorophenyl)piperazin-1-yl)cyclohexyl)-4-((5- |
| | fluoro-4-(3-(2-oxopiperidin-1-yl)phenyl)pyrimidin-2-yl)amino)cyclohexane-1-carboxamide |
| 565 | 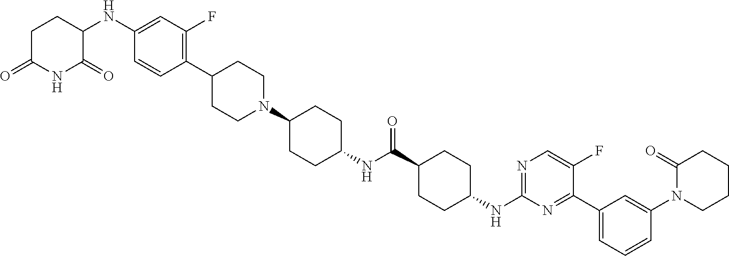 [SMILES: O=C1CCC(Nc2ccc(C3CCN([C@H]4CC[C@H](NC(=O)[C@H]5CC[C@H](Nc6ncc(F)c(-c7cccc(N8CCCCC8=O)c7)n6)CC5)CC4)CC3)c(F)c2)C(=O)N1] |
| | trans-N-(trans-4-(4-(4-((2,6-dioxopiperidin-3-yl)amino)-2-fluorophenyl)piperidin-1-yl)cyclohexyl)-4-((5-fluoro-4- |
| | (3-(2-oxopiperidin-1-yl)phenyl)pyrimidin-2-yl)amino)cyclohexane-1-carboxamide |
| 566 | 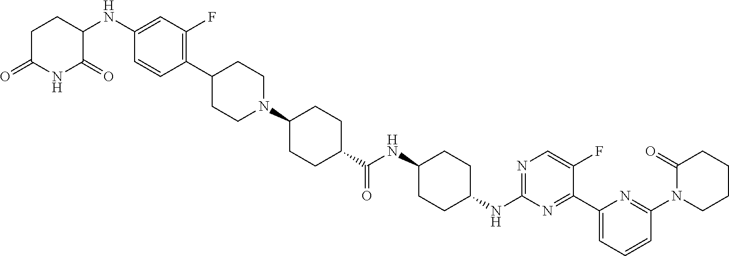 [SMILES: O=C1CCC(Nc2ccc(C3CCN([C@H]4CC[C@H](C(=O)N[C@H]5CC[C@H](Nc6ncc(F)c(-c7cccc(N8CCCCC8=O)n7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4-(6- |
| | (2-oxopiperidin-1-yl)pyridin-2-yl)pyrimidin-2-yl)amino)cyclohexyl)cyclohexane-1-carboxamide |
| 567 | 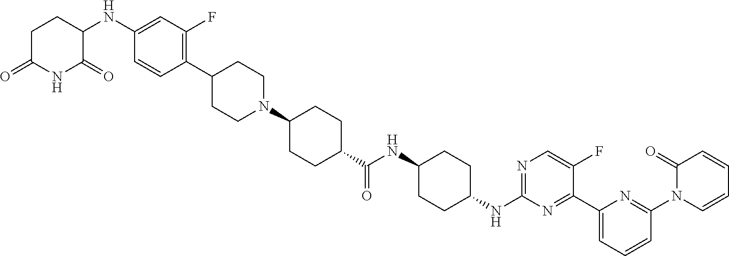 [SMILES: O=C1CCC(Nc2ccc(C3CCN([C@H]4CC[C@H](C(=O)N[C@H]5CC[C@H](Nc6ncc(F)c(-c7cccc(-n8ccccc8=O)n7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4-(2- |
| | oxo-2H-[1,2′-bipyridin]-6′-yl)pyrimidin-2-yl)amino)cyclohexyl)cyclohexane-1-carboxamide |
| 568 | 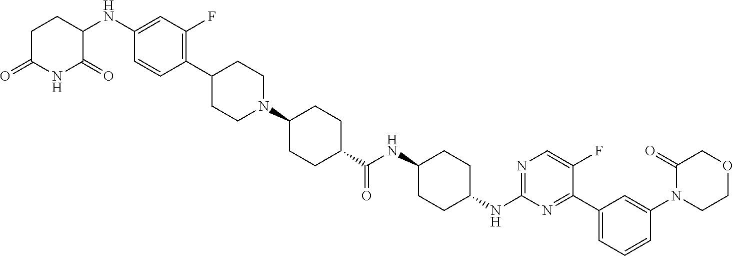 [SMILES: O=C1CCC(Nc2ccc(C3CCN([C@H]4CC[C@H](C(=O)N[C@H]5CC[C@H](Nc6ncc(F)c(-c7cccc(N8CCOCC8=O)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4-(3- |
| | (3-oxomorpholino)phenyl)pyrimidin-2-yl)amino)cyclohexyl)cyclohexane-1-carboxamide |
| 569 | 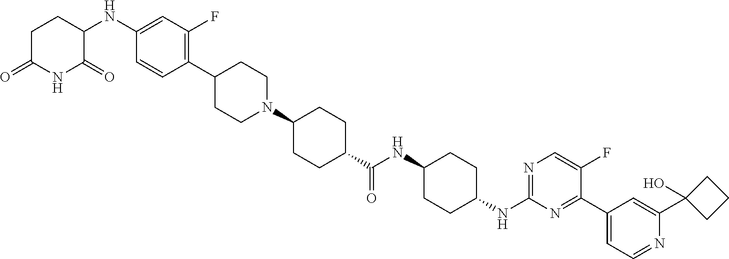 [SMILES: O=C1CCC(Nc2ccc(C3CCN([C@H]4CC[C@H](C(=O)N[C@H]5CC[C@H](Nc6ncc(F)c(-c7ccnc(C8(O)CCC8)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4- |
| | (2-(1-hydroxycyclobutyl)pyridin-4-yl)pyrimidin-2-yl)amino)cyclohexyl)cyclohexane-1-carboxamide |
| 570 | 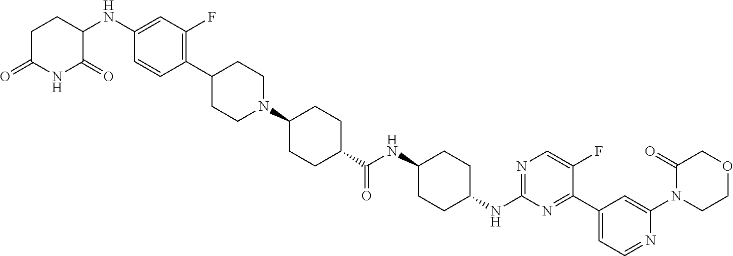 [SMILES: O=C1CCC(Nc2ccc(C3CCN([C@H]4CC[C@H](C(=O)N[C@H]5CC[C@H](Nc6ncc(F)c(-c7ccnc(N8CCOCC8=O)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4- |
| | (2-(3-oxomorpholino)pyridin-4-yl)pyrimidin-2-yl)amino)cyclohexyl)cyclohexane-1-carboxamide |
| 571 | 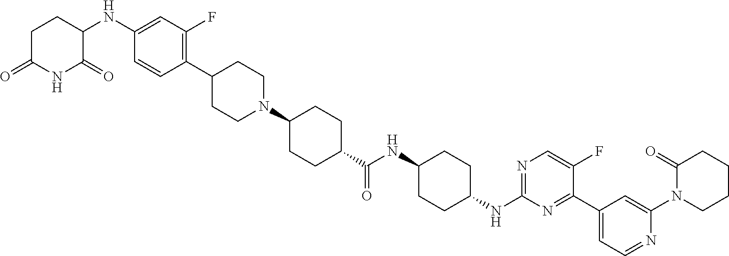 [SMILES: O=C1CCC(Nc2ccc(C3CCN([C@H]4CC[C@H](C(=O)N[C@H]5CC[C@H](Nc6ncc(F)c(-c7ccnc(N8CCCCC8=O)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4-(2- |
| | (2-oxopiperidin-1-yl)pyridin-4-yl)pyrimidin-2-yl)amino)cyclohexyl)cyclohexane-1-carboxamide |
| 572 | 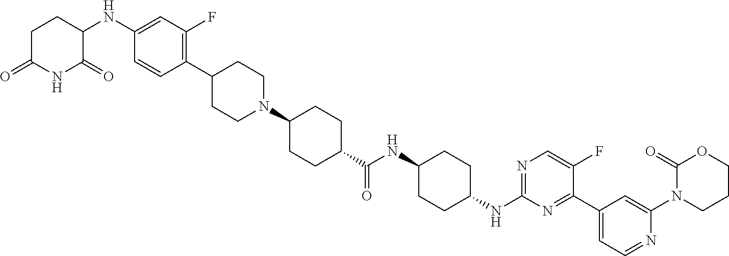 [SMILES: O=C1CCC(Nc2ccc(C3CCN([C@H]4CC[C@H](C(=O)N[C@H]5CC[C@H](Nc6ncc(F)c(-c7ccnc(N8CCCOC8=O)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4-(2- |
| | (2-oxo-1,3-oxazinan-3-yl)pyridin-4-yl)pyrimidin-2-yl)amino)cyclohexyl)cyclohexane-1-carboxamide |
| 573 | 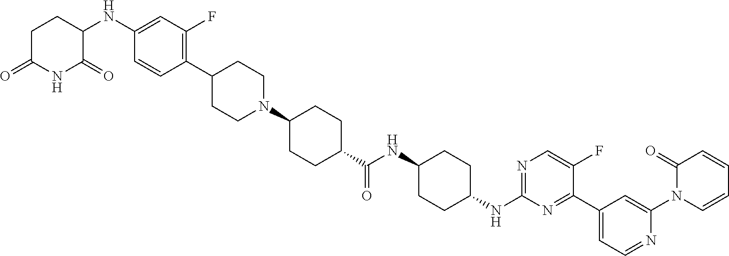 [SMILES: O=C1CCC(Nc2ccc(C3CCN([C@H]4CC[C@H](C(=O)N[C@H]5CC[C@H](Nc6ncc(F)c(-c7ccnc(-n8ccccc8=O)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4-(2- |
| | oxo-2H-[1,2′-bipyridin]-4′-yl)pyrimidin-2-yl)amino)cyclohexyl)cyclohexane-1-carboxamide |
| 574 | 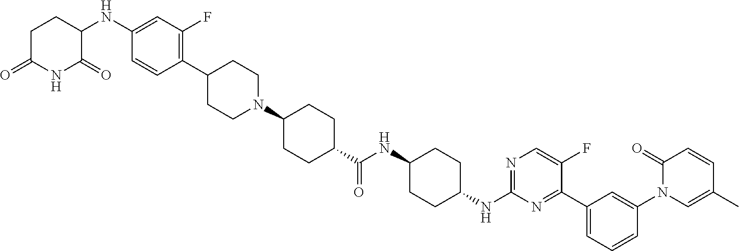 [SMILES: Cc1ccc(=O)n(-c2cccc(-c3nc(N[C@H]4CC[C@H](NC(=O)[C@H]5CC[C@H](N6CCC(c7ccc(NC8CCC(=O)NC8=O)cc7F)CC6)CC5)CC4)ncc3F)c2)c1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4-(3-(5- |
| | methyl-2-oxopyridin-1(2H)-yl)phenyl)pyrimidin-2-yl)amino)cyclohexyl)cyclohexane-1-carboxamide |
| 575 | 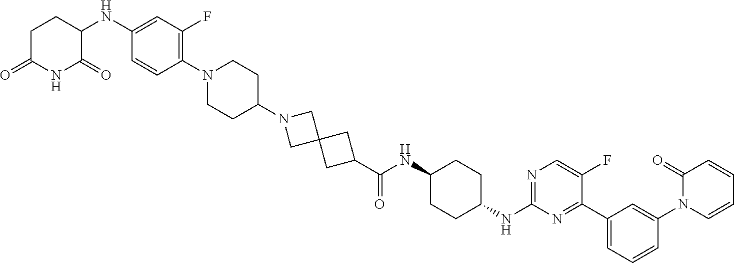 [SMILES: O=C1CCC(Nc2ccc(N3CCC(N4CC5(CC(C(=O)N[C@H]6CC[C@H](Nc7ncc(F)c(-c8cccc(-n9ccccc9=O)c8)n7)CC6)C5)C4)CC3)c(F)c2)C(=O)N1] |
| | trans-2-(1-(4-((2,6-dioxopiperidin-3-yl)amino)-2-fluorophenyl)piperidin-4-yl)-N-((1r,4r)-4-((5-fluoro-4-(3-(2-oxo |
| | pyridin-1(2H)-yl)phenyl)pyrimidin-2-yl)amino)cyclohexyl)-2-azaspiro[3.3]heptane-6-carboxamide |
| 576 | 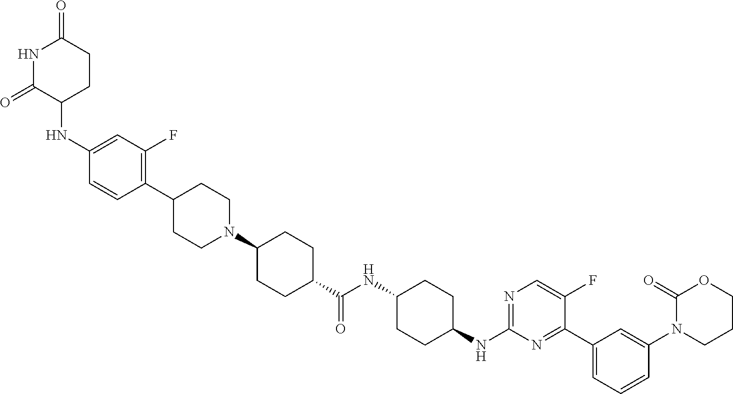 [SMILES: O=C1CCC(Nc2ccc(C3CCN([C@H]4CC[C@H](C(=O)N[C@H]5CC[C@H](Nc6ncc(F)c(-c7cccc(N8CCCOC8=O)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4- |
| | (3-(2-oxo-1,3-oxazinan-3-yl)phenyl)pyrimidin-2-yl)amino)cyclohexyl)cyclohexane-1-carboxamide |
| 577 | 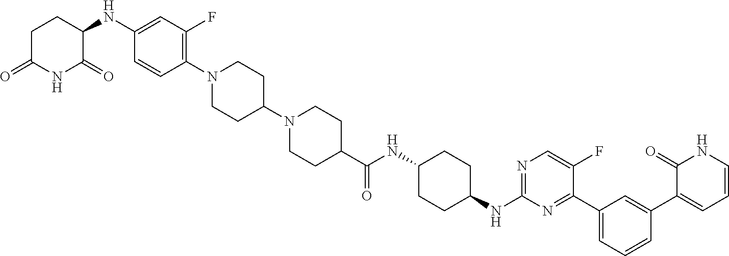 [SMILES: O=C1CC[C@@H](Nc2ccc(N3CCC(N4CCC(C(=O)N[C@H]5CC[C@H](Nc6ncc(F)c(-c7cccc(-c8ccc[nH]c8=O)c7)n6)CC5)CC4)CC3)c(F)c2)C(=O)N1] |
| | trans-1′-(4-(((R)-2,6-dioxopiperidin-3-yl)amino)-2-fluorophenyl)-N-((1r,4r)-4-((5-fluoro-4-(3-(2-oxo-1,2-dihydro- |
| | pyridin-3-yl)phenyl)pyrimidin-2-yl)amino)cyclohexyl)-[1,4′-bipiperidine]-4-carboxamide |
| 578 | 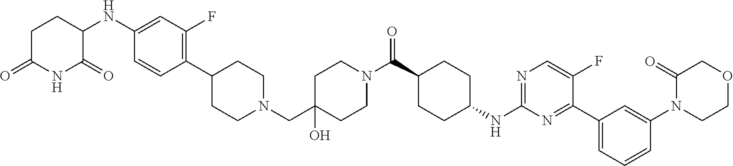 [SMILES: O=C1CCC(Nc2ccc(C3CCN(CC4(O)CCN(C(=O)[C@H]5CC[C@H](Nc6ncc(F)c(-c7cccc(N8CCOCC8=O)c7)n6)CC5)CC4)CC3)c(F)c2)C(=O)N1] |
| | trans-3-((3-fluoro-4-(1-((1-(4-((5-fluoro-4-(3-(3-oxomorpholino)phenyl)pyrimidin-2-yl)amino)cyclohexane-1- |
| | carbonyl)-4-hydroxypiperidin-4-yl)methyl)piperidin-4-yl)phenyl)amino)piperidine-2,6-dione |
| 579 | 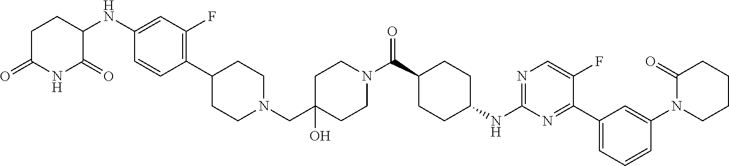 [SMILES: O=C1CCC(Nc2ccc(C3CCN(CC4(O)CCN(C(=O)[C@H]5CC[C@H](Nc6ncc(F)c(-c7cccc(N8CCCCC8=O)c7)n6)CC5)CC4)CC3)c(F)c2)C(=O)N1] |
| | 3-((3-fluoro-4-(1-((1-(trans-4-((5-fluoro-4-(3-(2-oxopiperidin-1-yl)phenyl)pyrimidin-2-yl)amino)cyclohexane-1- |
| | carbonyl)-4-hydroxypiperidin-4-yl)methyl)piperidin-4-yl)phenyl)amino)piperidine-2,6-dione |
| 580 | 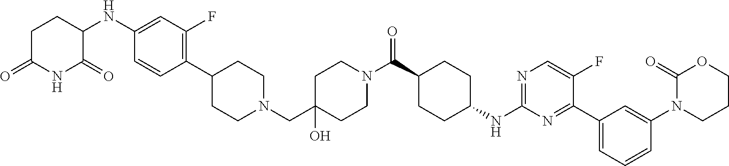 [SMILES: O=C1CCC(Nc2ccc(C3CCN(CC4(O)CCN(C(=O)[C@H]5CC[C@H](Nc6ncc(F)c(-c7cccc(N8CCCOC8=O)c7)n6)CC5)CC4)CC3)c(F)c2)C(=O)N1] |
| | trans-3-((3-fluoro-4-(1-((1-(4-((5-fluoro-4-(3-(2-oxo-1,3-oxazinan-3-yl)phenyl)pyrimidin-2-yl)amino)cyclohexane-1- |
| | carbonyl)-4-hydroxypiperidin-4-yl)methyl)piperidin-4-yl)phenyl)amino)piperidine-2,6-dione |
| 581 | 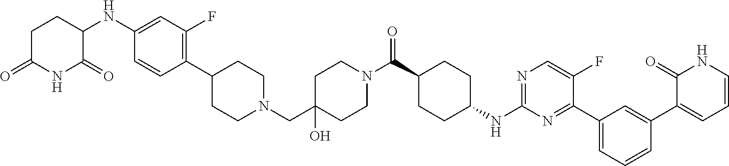 [SMILES: O=C1CCC(Nc2ccc(C3CCN(CC4(O)CCN(C(=O)[C@H]5CC[C@H](Nc6ncc(F)c(-c7cccc(-c8ccc[nH]c8=O)c7)n6)CC5)CC4)CC3)c(F)c2)C(=O)N1] |
| | 3-((3-fluoro-4-(1-((1-(trans-4-((5-fluoro-4-(3-(2-oxo-1,2-dihydropyridin-3-yl)phenyl)pyrimidin-2-yl)amino)cyclo- |
| | hexane-1-carbonyl)-4-hydroxypiperidin-4-yl)methyl)piperidin-4-yl)phenyl)amino)piperidine-2,6-dione |
| 582 | 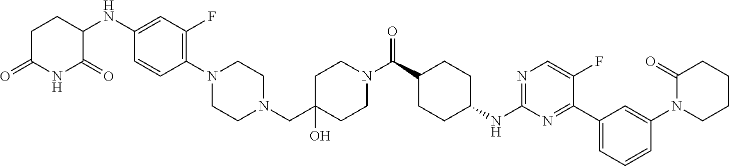 |
| | trans-3-((3-fluoro-4-(4-((1-(4-((5-fluoro-4-(3-(2-oxopiperidin-1-yl)phenyl)pyrimidin-2-yl)amino)cyclohexane-1- |
| | carbonyl)-4-hydroxypiperidin-4-yl)methyl)piperazin-1-yl)phenyl)amino)piperidine-2,6-dione |
| 583 | 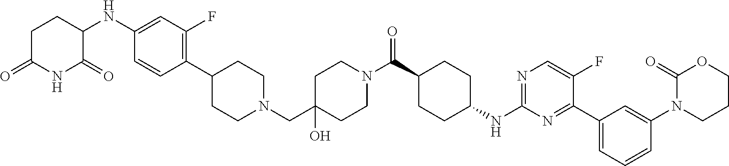 [SMILES: O=C1CCC(Nc2ccc(C3CCN(CC4(O)CCN(C(=O)[C@H]5CC[C@H](Nc6ncc(F)c(-c7cccc(N8CCCOC8=O)c7)n6)CC5)CC4)CC3)c(F)c2)C(=O)N1] |
| | trans-3-((3-fluoro-4-(4-((1-(4-((5-fluoro-4-(3-(2-oxo-1,3-oxazinan-3-yl)phenyl)pyrimidin-2-yl)amino)cyclohexane-1- |
| | carbonyl)-4-hydroxypiperidin-4-yl)methyl)piperazin-1-yl)phenyl)amino)piperidine-2,6-dione |
| 584 | 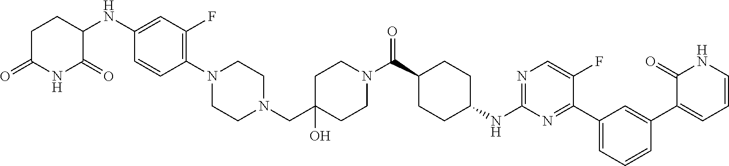 [SMILES: O=C1CCC(Nc2ccc(N3CCN(CC4(O)CCN(C(=O)[C@H]5CC[C@H](Nc6ncc(F)c(-c7cccc(-c8ccc[nH]c8=O)c7)n6)CC5)CC4)CC3)c(F)c2)C(=O)N1] |
| | trans-3-((3-fluoro-4-(4-((1-(4-((5-fluoro-4-(3-(2-oxo-1,2-dihydropyridin-3-yl)phenyl)pyrimidin-2-yl)amino)cyclo- |
| | hexane-1-carbonyl)-4-hydroxypiperidin-4-yl)methyl)piperazin-1-yl)phenyl)amino)piperidine-2,6-dione |
| 585 | 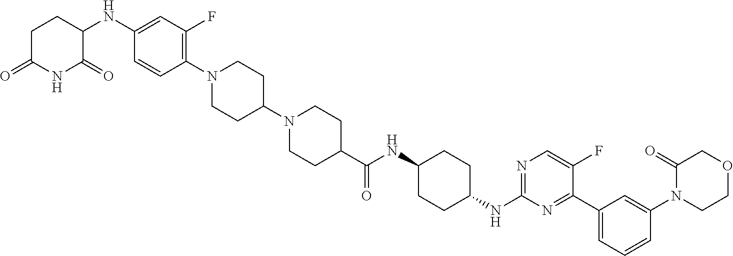 [SMILES: O=C1CCC(Nc2ccc(N3CCC(N4CCC(C(=O)N[C@H]5CC[C@H](Nc6ncc(F)c(-c7cccc(N8CCOCC8=O)c7)n6)CC5)CC4)CC3)c(F)c2)C(=O)N1] |
| | trans-1′-(4-((2,6-dioxopiperidin-3-yl)amino)-2-fluorophenyl)-N-(4-((5-fluoro-4-(3-(3-oxomorpholino)phenyl)pyrimidin- |
| | 2-yl)amino)cyclohexyl)-[1,4′-bipiperidine]-4-carboxamide |
| 586 | 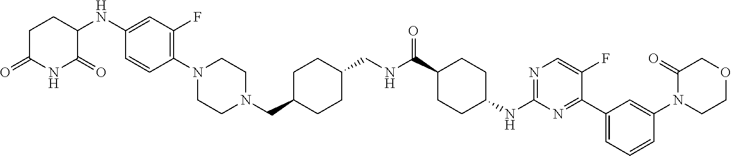 [SMILES: O=C1CCC(Nc2ccc(N3CCN(C[C@H]4CC[C@H](CNC(=O)[C@H]5CC[C@H](Nc6ncc(F)c(-c7cccc(N8CCOCC8=O)c7)n6)CC5)CC4)CC3)c(F)c2)C(=O)N1] |
| | trans-(1r,4R)-N-(((1r,4R)-4-((4-(4-((2,6-dioxopiperidin-3-yl)amino)-2-fluorophenyl)piperazin-1-yl)methyl)cyclo- |
| | hexyl)methyl)-4-((5-fluoro-4-(3-(3-oxomorpholino)phenyl)pyrimidin-2-yl)amino)cyclohexane-1-carboxamide |
| 587 | 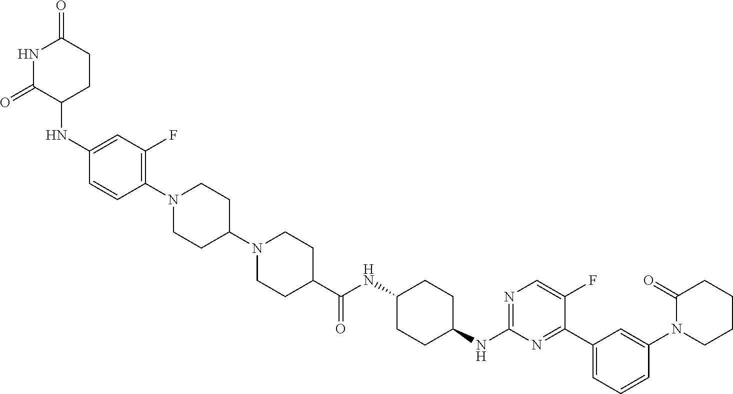 [SMILES: O=C1CCC(Nc2ccc(N3CCC(N4CCC(C(=O)N[C@H]5CC[C@H](Nc6ncc(F)c(-c7cccc(N8CCCCC8=O)c7)n6)CC5)CC4)CC3)c(F)c2)C(=O)N1] |
| | 1′-(4-((2,6-dioxopiperidin-3-yl)amino)-2-fluorophenyl)-N-(trans-4-((5-fluoro-4-(3-(2-oxopiperidin-1-yl)phenyl)pyrimidin- |
| | 2-yl)amino)cyclohexyl)-[1,4′-bipiperidine]-4-carboxamide |
| 588 | 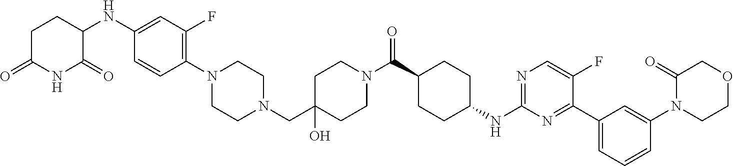 [SMILES: O=C1CCC(Nc2ccc(N3CCN(CC4(O)CCN(C(=O)[C@H]5CC[C@H](Nc6ncc(F)c(-c7cccc(N8CCOCC8=O)c7)n6)CC5)CC4)CC3)c(F)c2)C(=O)N1] |
| | trans-3-((3-fluoro-4-(4-((1-4-((5-fluoro-4-(3-(3-oxomorpholino)phenyl)pyrimidin-2-yl)amino)cyclohexane-1-carbonyl)- |
| | 4-hydroxypiperidin-4-yl)methyl)piperazin-1-yl)phenyl)amino)piperidine-2,6-dione |
| 589 | 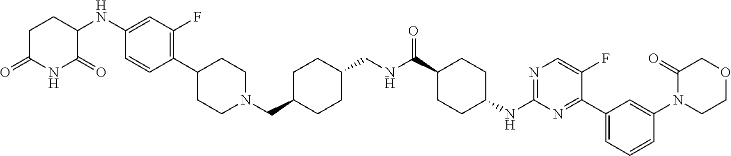 [SMILES: O=C1CCC(Nc2ccc(C3CCN(C[C@H]4CC[C@H](CNC(=O)[C@H]5CC[C@H](Nc6ncc(F)c(-c7cccc(N8CCOCC8=O)c7)n6)CC5)CC4)CC3)c(F)c2)C(=O)N1] |
| | trans-(1r,4R)-N-(((1r,4R)-4-((4-(4-((2,6-dioxopiperidin-3-yl)amino)-2-fluorophenyl)piperidin-1-yl)methyl)cyclohexyl)- |
| | methyl)-4-((5-fluoro-4-(3-(3-oxomorpholino)phenyl)pyrimidin-2-yl)amino)cyclohexane-1-carboxamide |
| 590 | 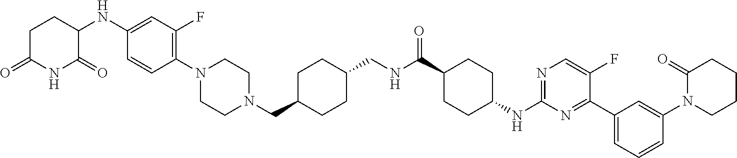 [SMILES: O=C1CCC(Nc2ccc(N3CCN(C[C@H]4CC[C@H](CNC(=O)[C@H]5CC[C@H](Nc6ncc(F)c(-c7cccc(N8CCCCC8=O)c7)n6)CC5)CC4)CC3)c(F)c2)C(=O)N1] |
| | trans-(1r,4R)-N-(((1r,4R)-4-((4-(4-((2,6-dioxopiperidin-3-yl)amino)-2-fluorophenyl)piperazin-1-yl)methyl)cyclohexyl)- |
| | methyl)-4-((5-fluoro-4-(3-(2-oxopiperidin-1-yl)phenyl)pyrimidin-2-yl)amino)cyclohexane-1-carboxamide |
| 591 | 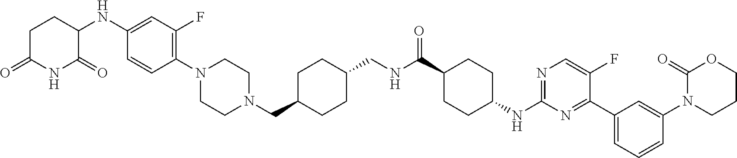 [SMILES: O=C1CCC(Nc2ccc(N3CCN(C[C@H]4CC[C@H](CNC(=O)[C@H]5CC[C@H](Nc6ncc(F)c(-c7cccc(N8CCCOC8=O)c7)n6)CC5)CC4)CC3)c(F)c2)C(=O)N1] |
| | trans-(1r,4R)-N-(((1r,4R)-4-((4-(4-((2,6-dioxopiperidin-3-yl)amino)-2-fluorophenyl)piperazin-1-yl)methyl)cyclohexyl)- |
| | methyl)-4-((5-fluoro-4-(3-(2-oxo-1,3-oxazinan-3-yl)phenyl)pyrimidin-2-yl)amino)cyclohexane-1-carboxamide |
| 592 | 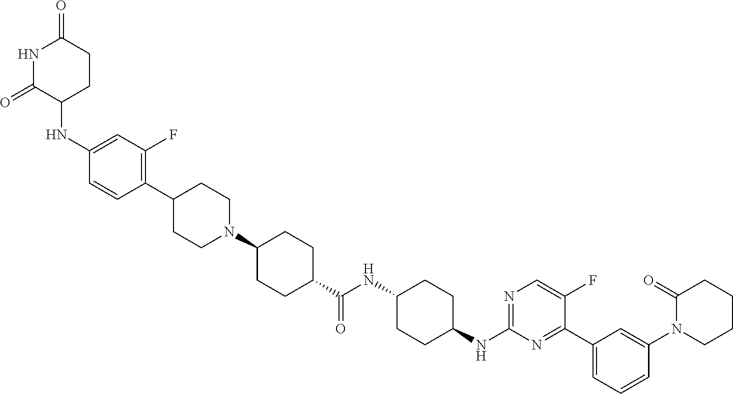 [SMILES: O=C1CCC(Nc2ccc(C3CCN([C@H]4CC[C@H](C(=O)N[C@H]5CC[C@H](Nc6ncc(F)c(-c7cccc(N8CCCCC8=O)c7)n6)CC5)CC4)CC3)c(F)c2)C(=O)N1] |
| | trans-(1S,4R)-4-(4-(4-(((S)-2,6-dioxopiperidin-3-yl)amino)-2-fluorophenyl)piperidin-1-yl)-N-((1r,4R)-4-((5-fluoro- |
| | 4-(3-(2-oxopiperidin-1-yl)phenyl)pyrimidin-2-yl)amino)cyclohexyl)cyclohexane-1-carboxamide |
| 593 | 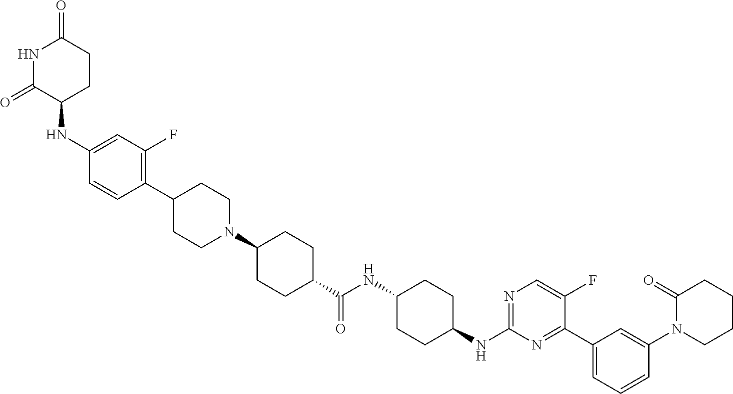 [SMILES: O=C1CC[C@@H](Nc2ccc(C3CCN([C@H]4CC[C@H](C(=O)N[C@H]5CC[C@H](Nc6ncc(F)c(-c7cccc(N8CCCCC8=O)c7)n6)CC5)CC4)CC3)c(F)c2)C(=O)N1] |
| | trans-(1R,4R)-4-(4-(4-(((R)-2,6-dioxopiperidin-3-yl)amino)-2-fluorophenyl)piperidin-1-yl)-N-((1r,4R)-4-((5-fluoro- |
| | 4-(3-(2-oxopiperidin-1-yl)phenyl)pyrimidin-2-yl)amino)cyclohexyl)cyclohexane-1-carboxamide |
| 594 | 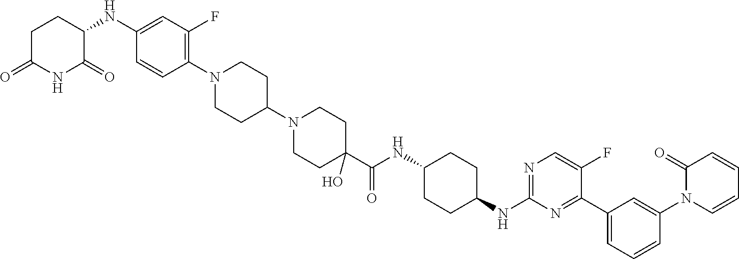 [SMILES: O=C1CC[C@H](Nc2ccc(N3CCC(N4CCC(O)(C(=O)N[C@H]5CC[C@H](Nc6ncc(F)c(-c7cccc(-n8ccccc8=O)c7)n6)CC5)CC4)CC3)c(F)c2)C(=O)N1] |
| | trans-(S)-1′-(4-((2,6-dioxopiperidin-3-yl)amino)-2-fluorophenyl)-N-(4-((5-fluoro-4-(3-(2-oxopyridin-1(2H)-yl)phenyl)- |
| | pyrimidin-2-yl)amino)cyclohexyl)-4-hydroxy-[1,4′-bipiperidine]-4-carboxamide |
| 595 | 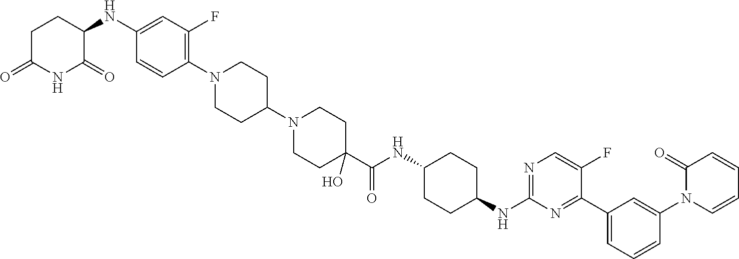 [SMILES: O=C1CC[C@@H](Nc2ccc(N3CCC(N4CCC(O)(C(=O)N[C@H]5CC[C@H](Nc6ncc(F)c(-c7cccc(-n8ccccc8=O)c7)n6)CC5)CC4)CC3)c(F)c2)C(=O)N1] |
| | trans-(R)-1′-(4-((2,6-dioxopiperidin-3-yl)amino)-2-fluorophenyl)-N-(4-((5-fluoro-4-(3-(2-oxopyridin-1(2H)-yl)phenyl)- |
| | pyrimidin-2-yl)amino)cyclohexyl)-4-hydroxy-[1,4′-bipiperidine]-4-carboxamide |
| 596 | 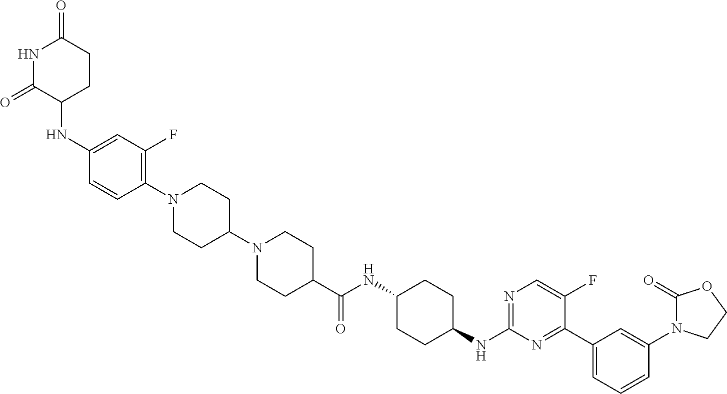 [SMILES: O=C1CCC(Nc2ccc(N3CCC(N4CCC(C(=O)N[C@H]5CC[C@H](Nc6ncc(F)c(-c7cccc(N8CCOC8=O)c7)n6)CC5)CC4)CC3)c(F)c2)C(=O)N1] |
| | trans-1′-(4-((2,6-dioxopiperidin-3-yl)amino)-2-fluorophenyl)-N-(4-((5-fluoro-4-(3-(2-oxooxazolidin-3-yl)phenyl)- |
| | pyrimidin-2-yl)amino)cyclohexyl)-[1,4′-bipiperidine]-4-carboxamide |
| 597 | 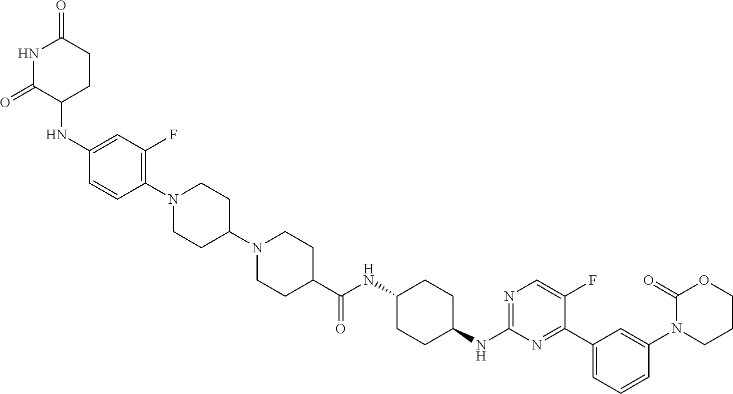 [SMILES: O=C1CCC(Nc2ccc(N3CCC(N4CCC(C(=O)N[C@H]5CC[C@H](Nc6ncc(F)c(-c7cccc(N8CCCOC8=O)c7)n6)CC5)CC4)CC3)c(F)c2)C(=O)N1] |
| | trans-1′-(4-((2,6-dioxopiperidin-3-yl)amino)-2-fluorophenyl)-N-(4-((5-fluoro-4-(3-(2-oxo-1,3-oxazinan-3-yl)phenyl)- |
| | pyrimidin-2-yl)amino)cyclohexyl)-[1,4′-bipiperidine]-4-carboxamide |
| 598 | 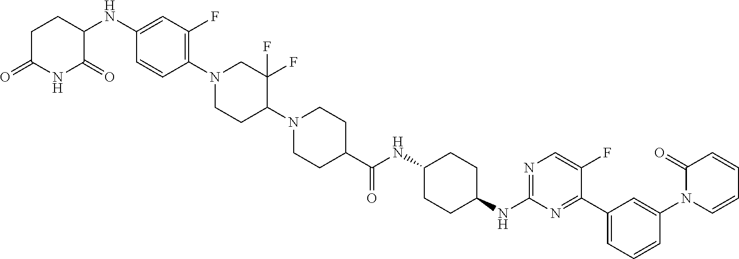 [SMILES: O=C1CCC(Nc2ccc(N3CCC(N4CCC(C(=O)N[C@H]5CC[C@H](Nc6ncc(F)c(-c7cccc(-n8ccccc8=O)c7)n6)CC5)CC4)C(F)(F)C3)c(F)c2)C(=O)N1] |
| | trans-1′-(4-((2,6-dioxopiperidin-3-yl)amino)-2-fluorophenyl)-3′,3′-difluoro-N-(4-((5-fluoro-4-(3-(2-oxopyridin-1(2H)- |
| | yl)phenyl)pyrimidin-2-yl)amino)cyclohexyl)-[1,4′-bipiperidine]-4-carboxamide |
| 599 | 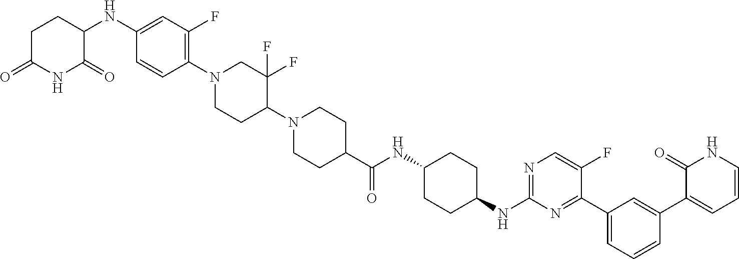 [SMILES: O=C1CCC(Nc2ccc(N3CCC(N4CCC(C(=O)N[C@H]5CC[C@H](Nc6ncc(F)c(-c7cccc(-c8ccc[nH]c8=O)c7)n6)CC5)CC4)C(F)(F)C3)c(F)c2)C(=O)N1] |
| | 1′-(4-((2,6-dioxopiperidin-3-yl)amino)-2-fluorophenyl)-3′,3′-difluoro-N-(trans-4-((5-fluoro-4-(3-(2-oxo-1,2-dihydro- |
| | pyridin-3-yl)phenyl)pyrimidin-2-yl)amino)cyclohexyl)-[1,4′-bipiperidine]-4-carboxamide |
| 600 | 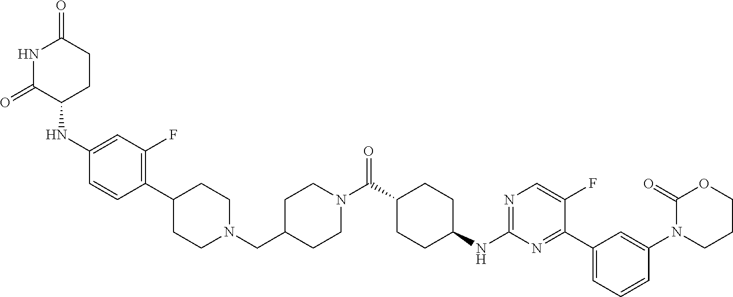 [SMILES: O=C1CC[C@H](Nc2ccc(C3CCN(CC4CCN(C(=O)[C@H]5CC[C@H](Nc6ncc(F)c(-c7cccc(N8CCCOC8=O)c7)n6)CC5)CC4)CC3)c(F)c2)C(=O)N1] |
| | trans-(S)-3-((3-fluoro-4-(1-((1-((1r,4r)-4-((5-fluoro-4-(3-(2-oxo-1,3-oxazinan-3-yl)phenyl)pyrimidin-2-yl)amino)cyclo- |
| | hexane-1-carbonyl)piperidin-4-yl)methyl)piperidin-4-yl)phenyl)amino)piperidine-2,6-dione |
| 601 | 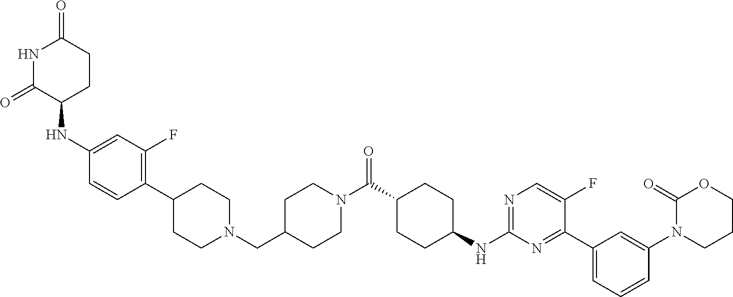 [SMILES: O=C1CC[C@@H](Nc2ccc(C3CCN(CC4CCN(C(=O)[C@H]5CC[C@H](Nc6ncc(F)c(-c7cccc(N8CCCOC8=O)c7)n6)CC5)CC4)CC3)c(F)c2)C(=O)N1] |
| | trans-(R)-3-((3-fluoro-4-(1-((1-((1r,4r)-4-((5-fluoro-4-(3-(2-oxo-1,3-oxazinan-3-yl)phenyl)pyrimidin-2-yl)amino)cyclo- |
| | hexane-1-carbonyl)piperidin-4-yl)methyl)piperidin-4-yl)phenyl)amino)piperidine-2,6-dione |
| 602 | 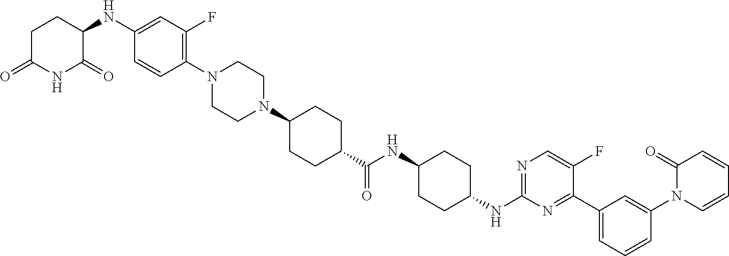 [SMILES: O=C1CC[C@@H](Nc2ccc(N3CCN([C@H]4CC[C@H](C(=O)N[C@H]5CC[C@H](Nc6ncc(F)c(-c7cccc(-n8ccccc8=O)c7)n6)CC5)CC4)CC3)c(F)c2)C(=O)N1] |
| | trans-(1R,4R)-4-(4-(4-(((R)-2,6-dioxopiperidin-3-yl)amino)-2-fluorophenyl)piperazin-1-yl)-N-((1r,4R)-4-((5-fluoro- |
| | 4-(3-(2-oxopyridin-1(2H)-yl)phenyl)pyrimidin-2-yl)amino)cyclohexyl)cyclohexane-1-carboxamide |
| 603 | 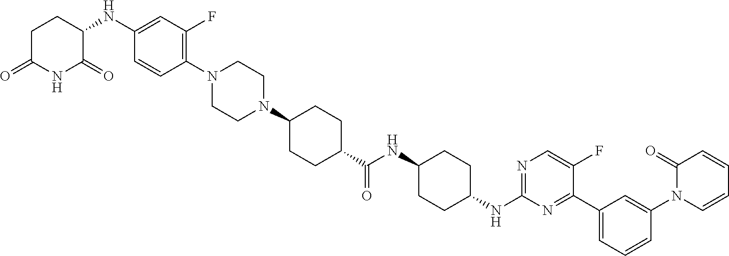 [SMILES: O=C1CC[C@H](Nc2ccc(N3CCN([C@H]4CC[C@H](C(=O)N[C@H]5CC[C@H](Nc6ncc(F)c(-c7cccc(-n8ccccc8=O)c7)n6)CC5)CC4)CC3)c(F)c2)C(=O)N1] |
| | trans-(1S,4R)-4-(4-(4-(((S)-2,6-dioxopiperidin-3-yl)amino)-2-fluorophenyl)piperazin-1-yl)-N-((1r,4R)-4-((5-fluoro- |
| | 4-(3-(2-oxopyridin-1(2H)-yl)phenyl)pyrimidin-2-yl)amino)cyclohexyl)cyclohexane-1-carboxamide |
| 604 | 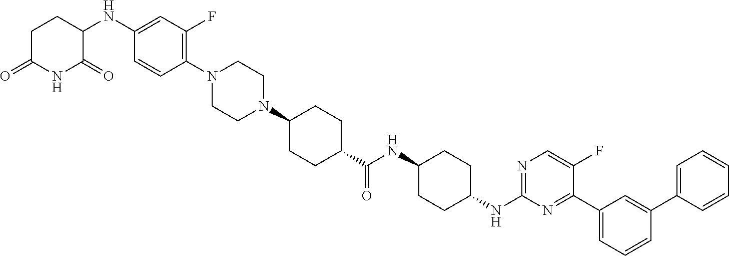 [SMILES: O=C1CCC(Nc2ccc(N3CCN([C@H]4CC[C@H](C(=O)N[C@H]5CC[C@H](Nc6ncc(F)c(-c7cccc(-c8ccccc8)c7)n6)CC5)CC4)CC3)c(F)c2)C(=O)N1] |
| | trans-(1r,4R)-N-((1r,4R)-4-((4-([1,1′-biphenyl]-3-yl)-5-fluoropyrimidin-2-yl)amino)cyclohexyl)-4-(4-(4-((2,6-dioxo- |
| | piperidin-3-yl)amino)-2-fluorophenyl)piperazin-1-yl)cyclohexane-1-carboxamide |
| 605 | 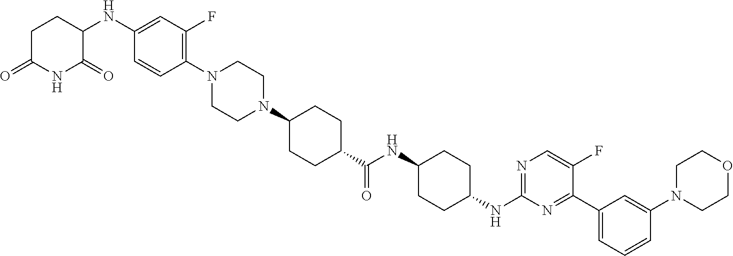 [SMILES: O=C1CCC(Nc2ccc(N3CCN([C@H]4CC[C@H](C(=O)N[C@H]5CC[C@H](Nc6ncc(F)c(-c7cccc(N8CCOCC8)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperazin-1-yl)-N-((1r,4R)-4-((5-fluoro-4-(3- |
| | morpholinophenyl)pyrimidin-2-yl)amino)cyclohexyl)cyclohexane-1-carboxamide |
| 632 | 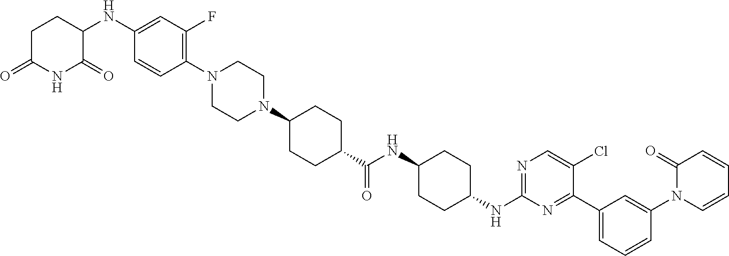 [SMILES: O=C1CCC(Nc2ccc(N3CCN([C@H]4CC[C@H](C(=O)N[C@H]5CC[C@H](Nc6ncc(Cl)c(-c7cccc(-n8ccccc8=O)c7)n6)CC5)CC4)CC3)c(F)c2)C(=O)N1] |
| | trans-(1r,4R)-N-((1r,4R)-4-((5-chloro-4-(3-(2-oxopyridin-1(2H)-yl)phenyl)pyrimidin-2-yl)amino)cyclohexyl)-4-(4-(4- |
| | ((2,6-dioxopiperidin-3-yl)amino)-2-fluorophenyl)piperazin-1-yl)cyclohexane-1-carboxamide |
| 633 | 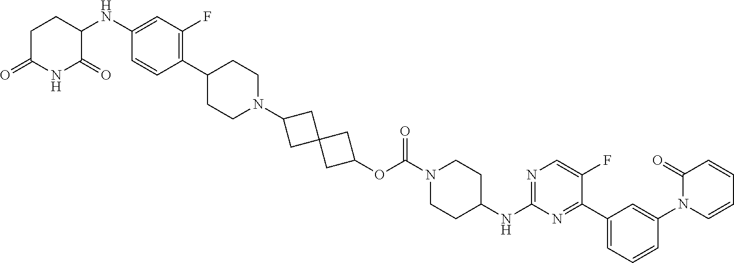 [SMILES: O=C1CCC(Nc2ccc(C3CCN(C4CC5(CC(OC(=O)N6CCC(Nc7ncc(F)c(-c8cccc(-n9ccccc9=O)c8)n7)CC6)C5)C4)CC3)c(F)c2)C(=O)N1] |
| | 6-(4-(4-((2,6-dioxopiperidin-3-yl)amino)-2-fluorophenyl)piperidin-1-yl)spiro[3.3]heptan-2-yl 4-((5-fluoro-4-(3-(2- |
| | oxopyridin-1(2H)-yl)phenyl)pyrimidin-2-yl)amino)piperidine-1-carboxylate |
| 634 | 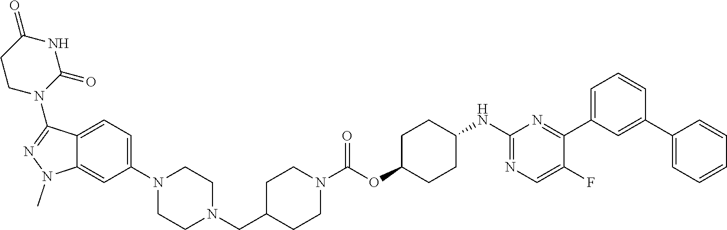 [SMILES: Cn1nc(N2CCC(=O)NC2=O)c2ccc(N3CCN(CC4CCN(C(=O)O[C@H]5CC[C@H](Nc6ncc(F)c(-c7cccc(-c8ccccc8)c7)n6)CC5)CC4)CC3)cc21] |
| | trans-4-((4-([1,1′-biphenyl]-3-yl)-5-fluoropyrimidin-2-yl)amino)cyclohexyl 4-((4-(3-(2,4-dioxotetrahydropyrimidin- |
| | 1(2H)-yl)-1-methyl-1H-indazol-6-yl)piperazin-1-yl)methyl)piperidine-1-carboxylate |
| 635 | 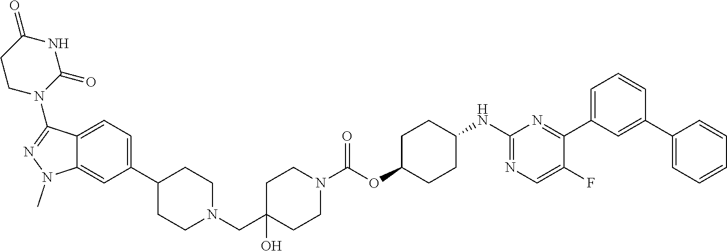 [SMILES: Cn1nc(N2CCC(=O)NC2=O)c2ccc(C3CCN(CC4(O)CCN(C(=O)O[C@H]5CC[C@H](Nc6ncc(F)c(-c7cccc(-c8ccccc8)c7)n6)CC5)CC4)CC3)cc21] |
| | trans-4-((4-([1,1′-biphenyl]-3-yl)-5-fluoropyrimidin-2-yl)amino)cyclohexyl 4-((4-(3-(2,4-dioxotetrahydropyrimidin- |
| | 1(2H)-yl)-1-methyl-1H-indazol-6-yl)piperidin-1-yl)methyl)-4-hydroxypiperidine-1-carboxylate |
| 636 | 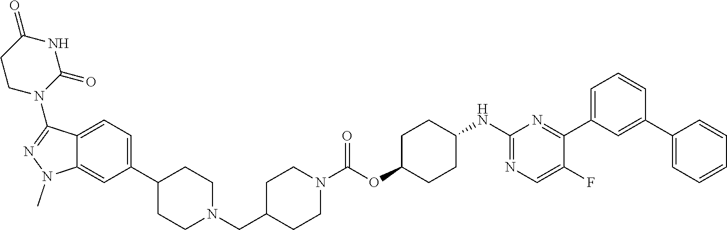 [SMILES: Cn1nc(N2CCC(=O)NC2=O)c2ccc(C3CCN(CC4CCN(C(=O)O[C@H]5CC[C@H](Nc6ncc(F)c(-c7cccc(-c8ccccc8)c7)n6)CC5)CC4)CC3)cc21] |
| | trans-4-((4-([1,1′-biphenyl]-3-yl)-5-fluoropyrimidin-2-yl)amino)cyclohexyl 4-((4-(3-(2,4-dioxotetrahydropyrimidin- |
| | 1(2H)-yl)-1-methyl-1H-indazol-6-yl)piperidin-1-yl)methyl)piperidine-1-carboxylate |
| 637 | 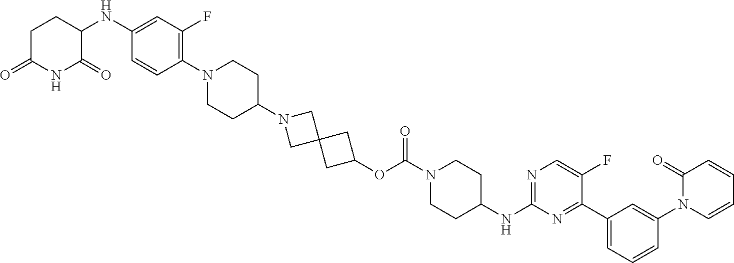 [SMILES: O=C1CCC(Nc2ccc(N3CCC(N4CC5(CC(OC(=O)N6CCC(Nc7ncc(F)c(-c8cccc(-n9ccccc9=O)c8)n7)CC6)C5)C4)CC3)c(F)c2)C(=O)N1] |
| | 2-(1-(4-((2,6-dioxopiperidin-3-yl)amino)-2-fluorophenyl)piperidin-4-yl)-2-azaspiro[3.3]heptan-6-yl 4-((5-fluoro-4-(3- |
| | (2-oxopyridin-1(2H)-yl)phenyl)pyrimidin-2-yl)amino)piperidine-1-carboxylate |
| 638 | 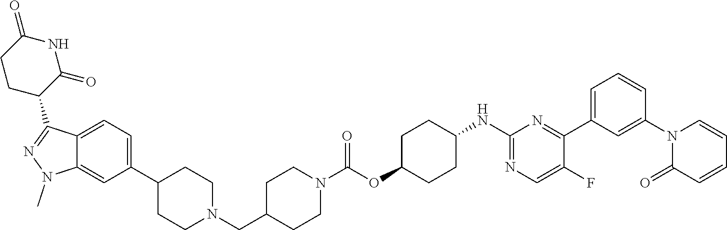 [SMILES: Cn1nc([C@@H]2CCC(=O)NC2=O)c2ccc(C3CCN(CC4CCN(C(=O)O[C@H]5CC[C@H](Nc6ncc(F)c(-c7cccc(-n8ccccc8=O)c7)n6)CC5)CC4)CC3)cc21] |
| | trans-4-((5-fluoro-4-(3-(2-oxopyridin-1(2H)-yl)phenyl)pyrimidin-2-yl)amino)cyclohexyl 4-((4-(3-((S)-2,6-dioxo- |
| | piperidin-3-yl)-1-methyl-1H-indazol-6-yl)piperidin-1-yl)methyl)piperidine-1-carboxylate |
| 639 | 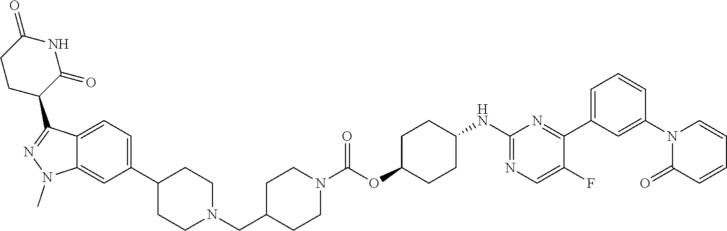 [SMILES: Cn1nc([C@H]2CCC(=O)NC2=O)c2ccc(C3CCN(CC4CCN(C(=O)O[C@H]5CC[C@H](Nc6ncc(F)c(-c7cccc(-n8ccccc8=O)c7)n6)CC5)CC4)CC3)cc21] |
| | trans-4-((5-fluoro-4-(3-(2-oxopyridin-1(2H)-yl)phenyl)pyrimidin-2-yl)amino)cyclohexyl 4-((4-(3-((R)-2,6-dioxo- |
| | piperidin-3-yl)-1-methyl-1H-indazol-6-yl)piperidin-1-yl)methyl)piperidine-1-carboxylate |
| 640 | 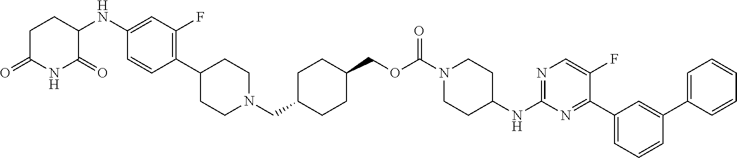 [SMILES: O=C1CCC(Nc2ccc(C3CCN(C[C@H]4CC[C@H](COC(=O)N5CCC(Nc6ncc(F)c(-c7cccc(-c8ccccc8)c7)n6)CC5)CC4)CC3)c(F)c2)C(=O)N1] |
| | trans-(4-((4-(4-((2,6-dioxopiperidin-3-yl)amino)-2-fluorophenyl)piperidin-1-yl)methyl)cyclohexyl)methyl 4-((4-([1,1′- |
| | biphenyl]-3-yl)-5-fluoropyrimidin-2-yl)amino)piperidine-1-carboxylate |
| 641 | 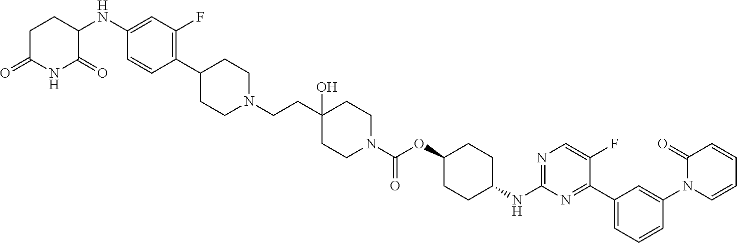 [SMILES: O=C1CCC(Nc2ccc(C3CCN(CCC4(O)CCN(C(=O)O[C@H]5CC[C@H](Nc6ncc(F)c(-c7cccc(-n8ccccc8=O)c7)n6)CC5)CC4)CC3)c(F)c2)C(=O)N1] |
| | trans-4-((5-fluoro-4-(3-(2-oxopyridin-1(2H)-yl)phenyl)pyrimidin-2-yl)amino)cyclohexyl 4-(2-(4-(4-((2,6-dioxopiperidin- |
| | 3-yl)amino)-2-fluorophenyl)piperidin-1-yl)ethyl)-4-hydroxypiperidine-1-carboxylate |
| 642 | 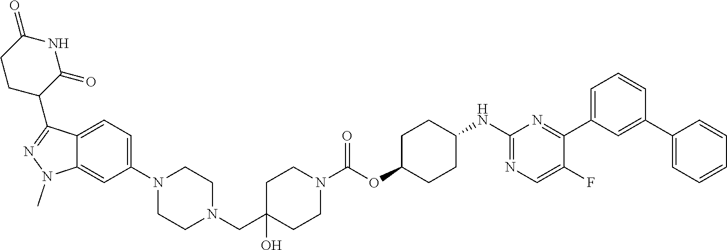 [SMILES: Cn1nc(C2CCC(=O)NC2=O)c2ccc(N3CCN(CC4(O)CCN(C(=O)O[C@H]5CC[C@H](Nc6ncc(F)c(-c7cccc(-c8ccccc8)c7)n6)CC5)CC4)CC3)cc21] |
| | trans-4-((4-([1,1′-biphenyl]-3-yl)-5-fluoropyrimidin-2-yl)amino)cyclohexyl 4-((4-(3-(2,6-dioxopiperidin-3-yl)-1-methyl- |
| | 1H-indazol-6-yl)piperazin-1-yl)methyl)-4-hydroxypiperidine-1-carboxylate |
| 643 | 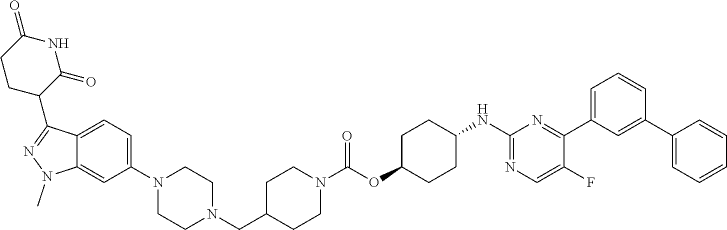 [SMILES: Cn1nc(C2CCC(=O)NC2=O)c2ccc(N3CCN(CC4CCN(C(=O)O[C@H]5CC[C@H](Nc6ncc(F)c(-c7cccc(-c8ccccc8)c7)n6)CC5)CC4)CC3)cc21] |
| | trans-4-((4-([1,1′-biphenyl]-3-yl)-5-fluoropyrimidin-2-yl)amino)cyclohexyl 4-((4-(3-(2,6-dioxopiperidin-3-yl)-1-methyl- |
| | 1H-indazol-6-yl)piperazin-1-yl)methyl)piperidine-1-carboxylate |
| 644 | 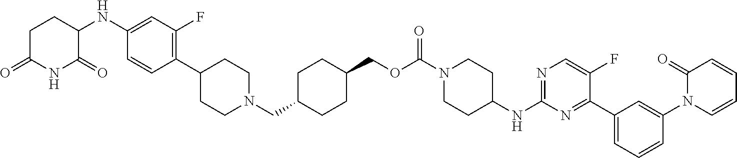 [SMILES: O=C1CCC(Nc2ccc(C3CCN(C[C@H]4CC[C@H](COC(=O)N5CCC(Nc6ncc(F)c(-c7cccc(-n8ccccc8=O)c7)n6)CC5)CC4)CC3)c(F)c2)C(=O)N1] |
| | trans-(4-((4-(4-((2,6-dioxopiperidin-3-yl)amino)-2-fluorophenyl)piperidin-1-yl)methyl)cyclohexyl)methyl 4-((5-fluoro- |
| | 4-(3-(2-oxopyridin-1(2H)-yl)phenyl)pyrimidin-2-yl)amino)piperidine-1-carboxylate |
| 645 | 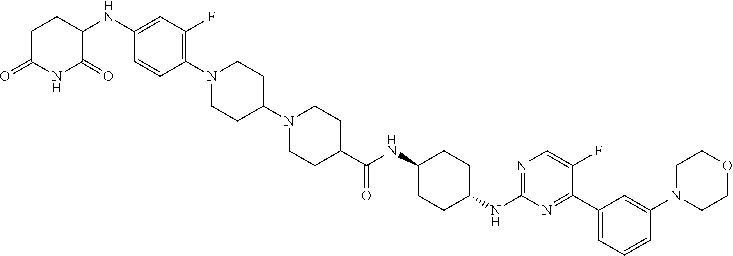 [SMILES: O=C1CCC(Nc2ccc(N3CCC(N4CCC(C(=O)N[C@H]5CC[C@H](Nc6ncc(F)c(-c7cccc(N8CCOCC8)c7)n6)CC5)CC4)CC3)c(F)c2)C(=O)N1] |
| | trans-1′-(4-((2,6-dioxopiperidin-3-yl)amino)-2-fluorophenyl)-N-(4-((5-fluoro-4-(3-morpholinophenyl)pyrimidin-2-yl)- |
| | amino)cyclohexyl)-[1,4′-bipiperidine]-4-carboxamide |
| 646 | 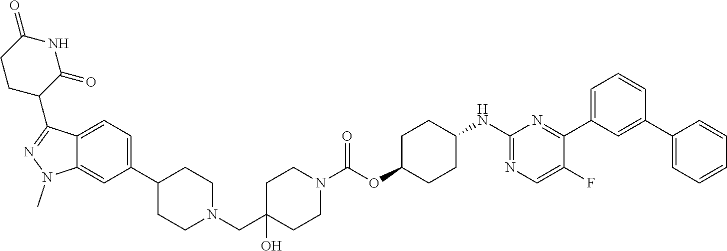 [SMILES: Cn1nc(C2CCC(=O)NC2=O)c2ccc(C3CCN(CC4(O)CCN(C(=O)O[C@H]5CC[C@H](Nc6ncc(F)c(-c7cccc(-c8ccccc8)c7)n6)CC5)CC4)CC3)cc21] |
| | trans-4-((4-([1,1′-biphenyl]-3-yl)-5-fluoropyrimidin-2-yl)amino)cyclohexyl 4-((4-(3-(2,6-dioxopiperidin-3-yl)-1-methyl- |
| | 1H-indazol-6-yl)piperidin-1-yl)methyl)-4-hydroxypiperidine-1-carboxylate |
| 647 | 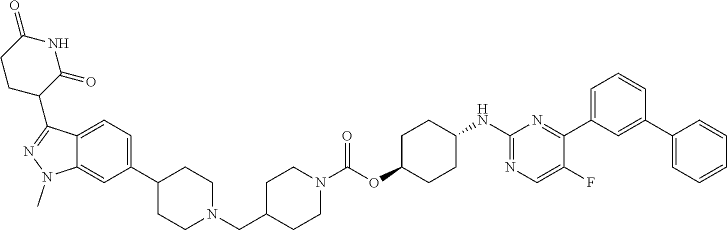 [SMILES: Cn1nc(C2CCC(=O)NC2=O)c2ccc(C3CCN(CC4CCN(C(=O)O[C@H]5CC[C@H](Nc6ncc(F)c(-c7cccc(-c8ccccc8)c7)n6)CC5)CC4)CC3)cc21] |
| | trans-4-((4-([1,1′-biphenyl]-3-yl)-5-fluoropyrimidin-2-yl)amino)cyclohexyl 4-((4-(3-(2,6-dioxopiperidin-3-yl)-1-methyl- |
| | 1H-indazol-6-yl)piperidin-1-yl)methyl)piperidine-1-carboxylate |
| 648 | 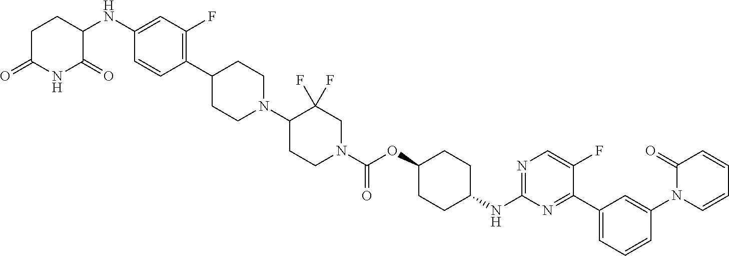 [SMILES: O=C1CCC(Nc2ccc(C3CCN(C4CCN(C(=O)O[C@H]5CC[C@H](Nc6ncc(F)c(-c7cccc(-n8ccccc8=O)c7)n6)CC5)CC4(F)F)CC3)c(F)c2)C(=O)N1] |
| | trans-4-((5-fluoro-4-(3-(2-oxopyridin-1(2H)-yl)phenyl)pyrimidin-2-yl)amino)cyclohexyl 4-(4-((2,6-dioxopiperidin- |
| | 3-yl)amino)-2-fluorophenyl)-3′,3′-difluoro-[1,4′-bipiperidine]-1′-carboxylate |
| 649 | 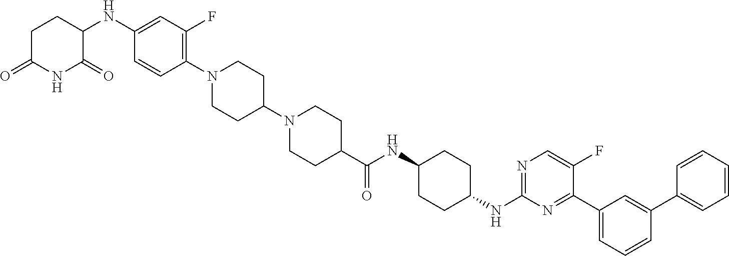 [SMILES: O=C1CCC(Nc2ccc(N3CCC(N4CCC(C(=O)N[C@H]5CC[C@H](Nc6ncc(F)c(-c7cccc(-c8ccccc8)c7)n6)CC5)CC4)CC3)c(F)c2)C(=O)N1] |
| | trans-N-(4-((4-([1,1′-biphenyl]-3-yl)-5-fluoropyrimidin-2-yl)amino)cyclohexyl)-1′-(4-((2,6-dioxopiperidin-3-yl)amino)- |
| | 2-fluorophenyl)-[1,4′-bipiperidine]-4-carboxamide |
| 650 | 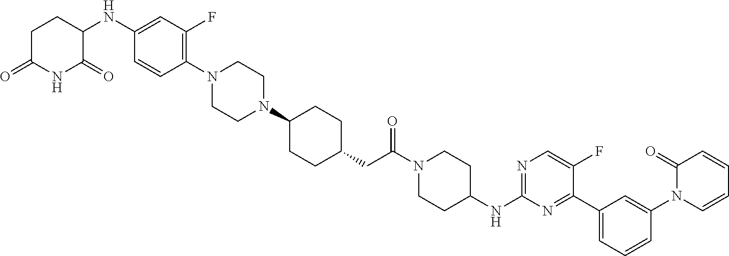 [SMILES: O=C1CCC(Nc2ccc(N3CCN([C@H]4CC[C@H](CC(=O)N5CCC(Nc6ncc(F)c(-c7cccc(-n8ccccc8=O)c7)n6)CC5)CC4)CC3)c(F)c2)C(=O)N1] |
| | trans-3-((3-fluoro-4-(4-(4-(2-(4-((5-fluoro-4-(3-(2-oxopyridin-1(2H)-yl)phenyl)pyrimidin-2-yl)amino)piperidin-1- |
| | yl)-2-oxoethyl)cyclohexyl)piperazin-1-yl)phenyl)amino)piperidine-2,6-dione |
| 651 | 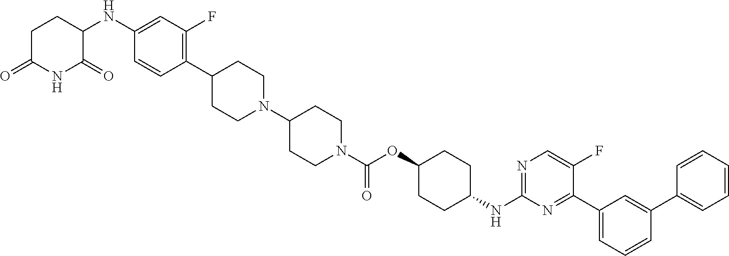 [SMILES: O=C1CCC(Nc2ccc(C3CCN(C4CCN(C(=O)O[C@H]5CC[C@H](Nc6ncc(F)c(-c7cccc(-c8ccccc8)c7)n6)CC5)CC4)CC3)c(F)c2)C(=O)N1] |
| | trans-4-((4-([1,1′-biphenyl]-3-yl)-5-fluoropyrimidin-2-yl)amino)cyclohexyl 4-(4-((2,6-dioxopiperidin-3-yl)amino)- |
| | 2-fluorophenyl)-[1,4′-bipiperidine]-1′-carboxylate |
| 652 | 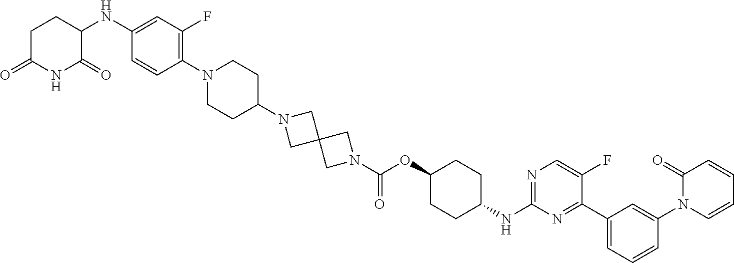 [SMILES: O=C1CCC(Nc2ccc(N3CCC(N4CC5(CN(C(=O)O[C@H]6CC[C@H](Nc7ncc(F)c(-c8cccc(-n9ccccc9=O)c8)n7)CC6)C5)C4)CC3)c(F)c2)C(=O)N1] |
| | (trans)-4-((5-fluoro-4-(3-(2-oxopyridin-1(2H)-yl)phenyl)pyrimidin-2-yl)amino)cyclohexyl 6-(1-(4-((2,6-dioxopiperidin- |
| | 3-yl)amino)-2-fluorophenyl)piperidin-4-yl)-2,6-diazaspiro[3.3]heptane-2-carboxylate |
| 653 | 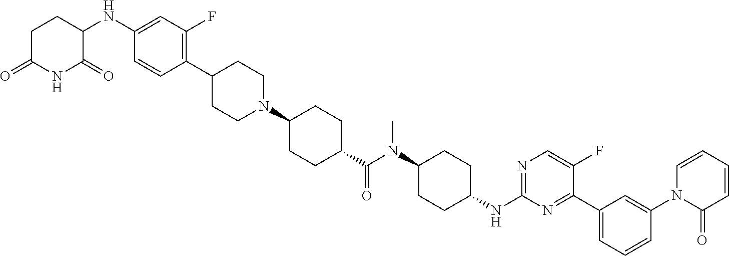 [SMILES: CN(C(=O)[C@H]1CC[C@H](N2CCC(c3ccc(NC4CCC(=O)NC4=O)cc3F)CC2)CC1)[C@H]1CC[C@H](Nc2ncc(F)c(-c3cccc(-n4ccccc4=O)c3)n2)CC1] |
| | trans-4-(4-(4-((2,6-dioxopiperidin-3-yl)amino)-2-fluorophenyl)piperidin-1-yl)-N-((trans)-4-((5-fluoro-4-(3-(2-oxo- |
| | pyridin-1(2H)-yl)phenyl)pyrimidin-2-yl)amino)cyclohexyl)-N-methylcyclohexane-1-carboxamide |
| 654 | 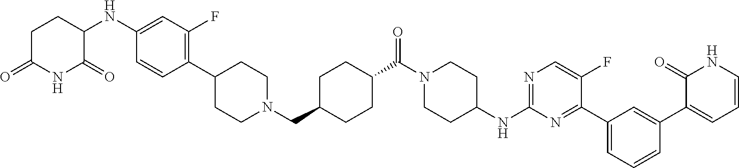 [SMILES: O=C1CCC(Nc2ccc(C3CCN(C[C@H]4CC[C@H](C(=O)N5CCC(Nc6ncc(F)c(-c7cccc(-c8ccc[nH]c8=O)c7)n6)CC5)CC4)CC3)c(F)c2)C(=O)N1] |
| | 3-((3-fluoro-4-(1-(((trans)-4-(4-((5-fluoro-4-(3-(2-oxo-1,2-dihydropyridin-3-yl)phenyl)pyrimidin-2-yl)amino)piperidine- |
| | 1-carbonyl)cyclohexyl)methyl)piperidin-4-yl)phenyl)amino)piperidine-2,6-dione |
| 655 | 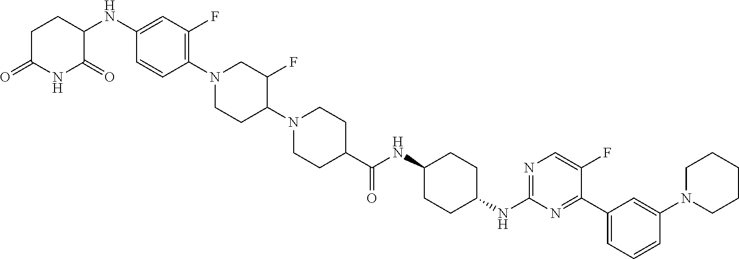 [SMILES: O=C1CCC(Nc2ccc(N3CCC(N4CCC(C(=O)N[C@H]5CC[C@H](Nc6ncc(F)c(-c7cccc(N8CCCCC8)c7)n6)CC5)CC4)C(F)C3)c(F)c2)C(=O)N1] |
| | 1′-(4-((2,6-dioxopiperidin-3-yl)amino)-2-fluorophenyl)-3′-fluoro-N-((1r,4r)-4-((5-fluoro-4-(3-morpholinophenyl)- |
| | pyrimidin-2-yl)amino)cyclohexyl)-[1,4′-bipiperidine]-4-carboxamide |
| 656 | 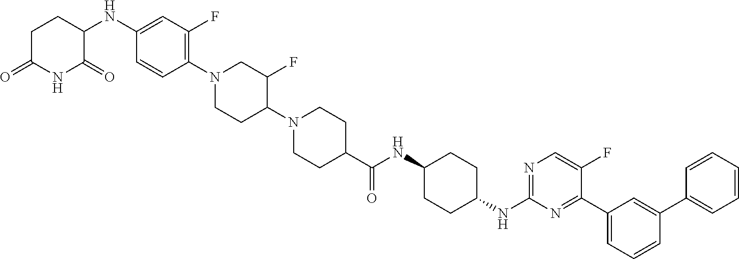 [SMILES: O=C1CCC(Nc2ccc(N3CCC(N4CCC(C(=O)N[C@H]5CC[C@H](Nc6ncc(F)c(-c7cccc(-c8ccccc8)c7)n6)CC5)CC4)C(F)C3)c(F)c2)C(=O)N1] |
| | N-((1r,4r)-4-((4-([1,1′-biphenyl]-3-yl)-5-fluoropyrimidin-2-yl)amino)cyclohexyl)-1′-(4-((2,6-dioxopiperidin-3-yl)amino)- |
| | 2-fluorophenyl)-3′-fluoro-[1,4′-bipiperidine]-4-carboxamide |
| 657 | 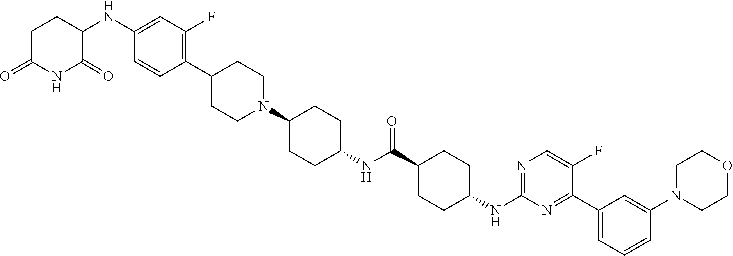 [SMILES: O=C1CCC(Nc2ccc(C3CCN([C@H]4CC[C@H](NC(=O)[C@H]5CC[C@H](Nc6ncc(F)c(-c7cccc(N8CCOCC8)c7)n6)CC5)CC4)CC3)c(F)c2)C(=O)N1] |
| | trans-(1r,4R)-N-((1r,4R)-4-(4-(4-((2,6-dioxopiperidin-3-yl)amino)-2-fluorophenyl)piperidin-1-yl)cyclohexyl)-4-((5- |
| | fluoro-4-(3-morpholinophenyl)pyrimidin-2-yl)amino)cyclohexane-1-carboxamide |
| 658 | 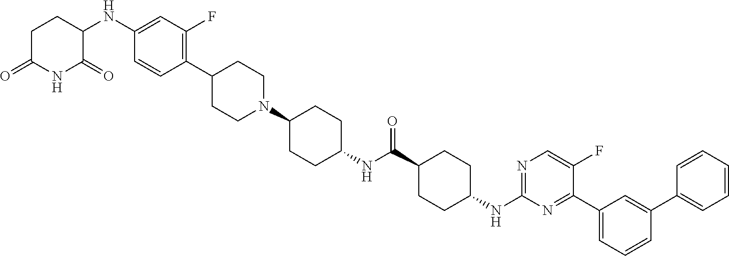 [SMILES: O=C1CCC(Nc2ccc(C3CCN([C@H]4CC[C@H](NC(=O)[C@H]5CC[C@H](Nc6ncc(F)c(-c7cccc(-c8ccccc8)c7)n6)CC5)CC4)CC3)c(F)c2)C(=O)N1] |
| | trans-(1r,4R)-4-((4-([1,1′-biphenyl]-3-yl)-5-fluoropyrimidin-2-yl)amino)-N-((1r,4R)-4-(4-(4-((2,6-dioxopiperidin-3- |
| | yl)amino)-2-fluorophenyl)piperidin-1-yl)cyclohexyl)cyclohexane-1-carboxamide |
| 659 | 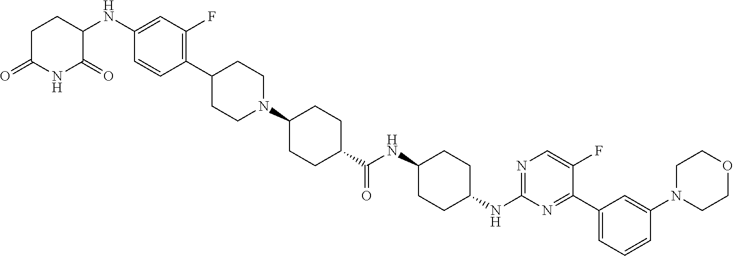 [SMILES: O=C1CCC(Nc2ccc(C3CCN([C@H]4CC[C@H](C(=O)N[C@H]5CC[C@H](Nc6ncc(F)c(-c7cccc(N8CCOCC8)c7)n6)CC5)CC4)CC3)c(F)c2)C(=O)N1] |
| | trans-(1r,4R)-4-(4-(4-((2,6-dioxopiperidin-3-yl)amino)-2-fluorophenyl)piperidin-1-yl)-N-((1r,4R)-4-((5-fluoro-4-(3- |
| | morpholinophenyl)pyrimidin-2-yl)amino)cyclohexyl)cyclohexane-1-carboxamide |
| 660 | 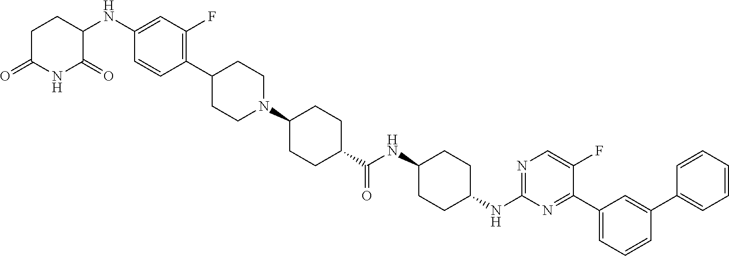 [SMILES: O=C1CCC(Nc2ccc(C3CCN([C@H]4CC[C@H](C(=O)N[C@H]5CC[C@H](Nc6ncc(F)c(-c7cccc(-c8ccccc8)c7)n6)CC5)CC4)CC3)c(F)c2)C(=O)N1] |
| | trans-(1r,4R)-N-((1r,4R)-4-((4-([1,1′-biphenyl]-3-yl)-5-fluoropyrimidin-2-yl)amino)cyclohexyl)-4-(4-(4-((2,6-dioxo- |
| | piperidin-3-yl)amino)-2-fluorophenyl)piperidin-1-yl)cyclohexane-1-carboxamide |
| 661 | 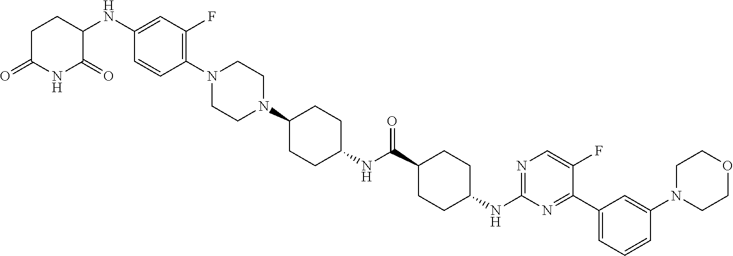 [SMILES: O=C1CCC(Nc2ccc(N3CCN([C@H]4CC[C@H](NC(=O)[C@H]5CC[C@H](Nc6ncc(F)c(-c7cccc(N8CCOCC8)c7)n6)CC5)CC4)CC3)c(F)c2)C(=O)N1] |
| | trans-(1r,4R)-N-((1r,4R)-4-(4-(4-((2,6-dioxopiperidin-3-yl)amino)-2-fluorophenyl)piperazin-1-yl)cyclohexyl)-4- |
| | ((5-fluoro-4-(3-morpholinophenyl)pyrimidin-2-yl)amino)cyclohexane-1-carboxamide |
| 662 | 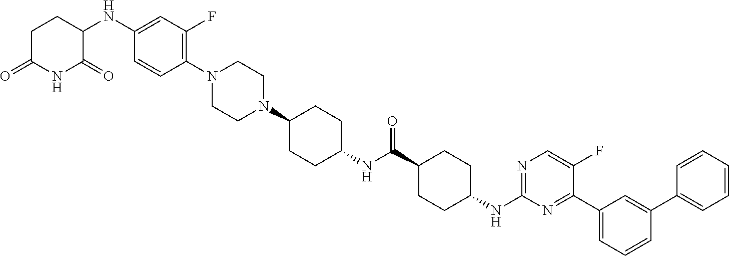 [SMILES: O=C1CCC(Nc2ccc(N3CCN([C@H]4CC[C@H](NC(=O)[C@H]5CC[C@H](Nc6ncc(F)c(-c7cccc(-c8ccccc8)c7)n6)CC5)CC4)CC3)c(F)c2)C(=O)N1] |
| | trans-(1r,4R)-4-((4-([1,1′-biphenyl]-3-yl)-5-fluoropyrimidin-2-yl)amino)-N-((1r,4R)-4-(4-(4-((2,6-dioxopiperidin-3-yl)- |
| | amino)-2-fluorophenyl)piperazin-1-yl)cyclohexyl)cyclohexane-1-carboxamide |
| 663 | 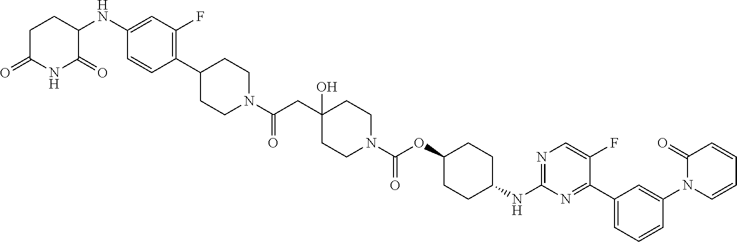 [SMILES: O=C1CCC(Nc2ccc(C3CCN(C(=O)CC4(O)CCN(C(=O)O[C@H]5CC[C@H](Nc6ncc(F)c(-c7cccc(-n8ccccc8=O)c7)n6)CC5)CC4)CC3)c(F)c2)C(=O)N1] |
| | trans-4-((5-fluoro-4-(3-(2-oxopyridin-1(2H)-yl)phenyl)pyrimidin-2-yl)amino)cyclohexyl 4-(2-(4-(4-((2,6-dioxo- |
| | piperidin-3-yl)amino)-2-fluorophenyl)piperidin-1-yl)-2-oxoethyl)-4-hydroxypiperidine-1-carboxylate |
| 664 | 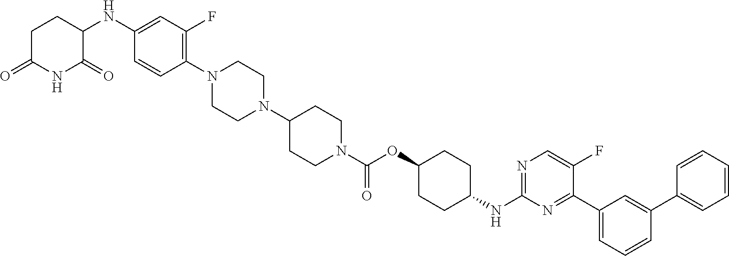 [SMILES: O=C1CCC(Nc2ccc(N3CCN(C4CCN(C(=O)O[C@H]5CC[C@H](Nc6ncc(F)c(-c7cccc(-c8ccccc8)c7)n6)CC5)CC4)CC3)c(F)c2)C(=O)N1] |
| | trans-[4-[[5-fluoro-4-(3-phenylphenyl)pyrimidin-2-yl]amino]cyclohexyl]4-[4-[4-[(2,6-dioxo-3-piperidyl)amino]-2-fluoro- |
| | phenyl]piperazin-1-yl]piperidine-1-carboxylate |
| 665 | 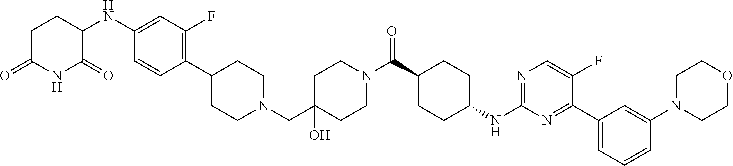 [SMILES: O=C1CCC(Nc2ccc(C3CCN(CC4(O)CCN(C(=O)[C@H]5CC[C@H](Nc6ncc(F)c(-c7cccc(N8CCOCC8)c7)n6)CC5)CC4)CC3)c(F)c2)C(=O)N1] |
| | 3-((3-fluoro-4-(1-((1-((trans)-4-((5-fluoro-4-(3-morpholinophenyl)pyrimidin-2-yl)amino)cyclohexane-1-carbonyl)-4- |
| | hydroxypiperidin-4-yl)methyl)piperidin-4-yl)phenyl)amino)piperidine-2,6-dione |
| 666 | 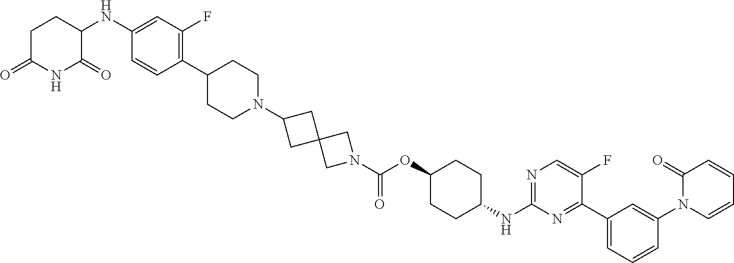 [SMILES: O=C1CCC(Nc2ccc(C3CCN(C4CC5(C4)CN(C(=O)O[C@H]4CC[C@H](Nc6ncc(F)c(-c7cccc(-n8ccccc8=O)c7)n6)CC4)C5)CC3)c(F)c2)C(=O)N1] |
| | (trans)-4-((5-fluoro-4-(3-(2-oxopyridin-1(2H)-yl)phenyl)pyrimidin-2-yl)amino)cyclohexyl 6-(4-(4-((2,6-dioxo- |
| | piperidin-3-yl)amino)-2-fluorophenyl)piperidin-1-yl)-2-azaspiro[3.3]heptane-2-carboxylate |
| 667 | 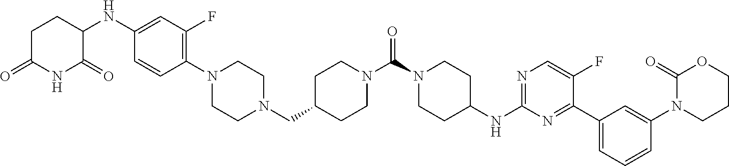 |
| | 3-((3-fluoro-4-(4-((trans-4-(4-((5-fluoro-4-(3-(2-oxo-1,3-oxazinan-3-yl)phenyl)pyrimidin-2-yl)amino)piperidine- |
| | 1-carbonyl)cyclohexyl)methyl)piperazin-1-yl)phenyl)amino)piperidine-2,6-dione |
| 668 | 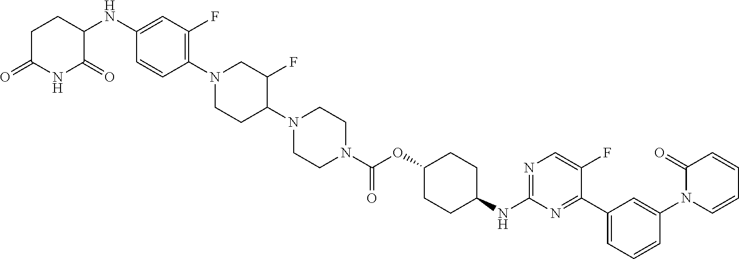 [SMILES: O=C1CCC(Nc2ccc(N3CCC(N4CCN(C(=O)O[C@H]5CC[C@H](Nc6ncc(F)c(-c7cccc(-n8ccccc8=O)c7)n6)CC5)CC4)C(F)C3)c(F)c2)C(=O)N1] |
| | trans-[4-[5-fluoro-4-[3-(2-oxo-1-pyridyl)phenyl]pyrimidin-2-yl]amino]cyclohexyl]4-[1-[4-[(2,6-dioxo-3-piperidyl)- |
| | amino]2-fluoro-phenyl]-3-fluoro-4-piperidyl]piperazine-1-carboxylate |
| 669 | 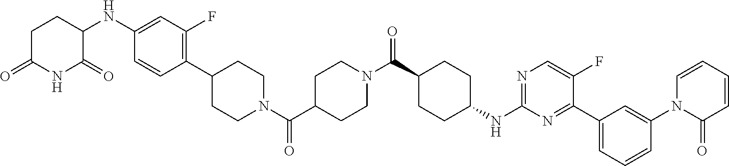 [SMILES: O=C1CCC(Nc2ccc(C3CCN(C(=O)C4CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-3-((3-fluoro-4-(1-(1-(4-((5-fluoro-4-(3-(2-oxopyridin-1(2H)-yl)phenyl)pyrimidin-2-yl)amino)cyclohexane-1- |
| | carbonyl)piperidine-4-carbonyl)piperidin-4-yl)phenyl)amino)piperidine-2,6-dione |
| 670 | 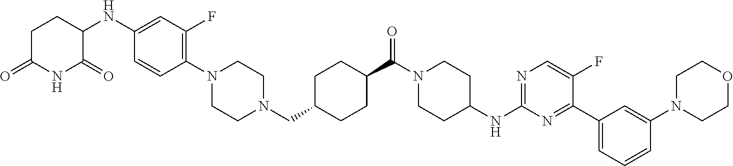 [SMILES: O=C1CCC(Nc2ccc(N3CCN(C[C@H]4CC[C@H](C(=O)N5CCC(Nc6ncc(F)c(-c7cccc(N8CCOCC8)c7)n6)CC5)CC4)CC3)c(F)c2)C(=O)N1] |
| | 3-((3-fluoro-4-(4-((trans-4-(4-((5-fluoro-4-(3-morpholinophenyl)pyrimidin-2-yl)amino)piperidine-1-carbonyl)cyclo- |
| | hexyl)methyl)piperazin-1-yl)phenyl)amino)piperidine-2,6-dione |
| 671 | 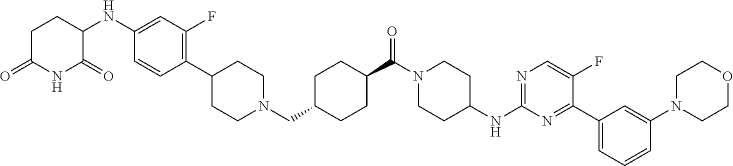 [SMILES: O=C1CCC(Nc2ccc(C3CCN(C[C@H]4CC[C@H](C(=O)N5CCC(Nc6ncc(F)c(-c7cccc(N8CCOCC8)c7)n6)CC5)CC4)CC3)c(F)c2)C(=O)N1] |
| | trans-3-((3-fluoro-4-(1-((4-(4-((5-fluoro-4-(3-morpholinophenyl)pyrimidin-2-yl)amino)piperidine-1-carbonyl)cyclo- |
| | hexyl)methyl)piperidin-4-yl)phenyl)amino)piperidine-2,6-dione |
| 672 | 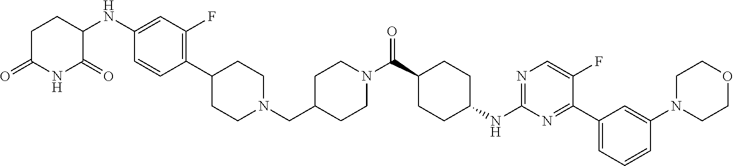 [SMILES: O=C1CCC(Nc2ccc(C3CCN(CC4CCN(C(=O)[C@H]5CC[C@H](Nc6ncc(F)c(-c7cccc(N8CCOCC8)c7)n6)CC5)CC4)CC3)c(F)c2)C(=O)N1] |
| | trans-3-((3-fluoro-4-(1-((1-(4-((5-fluoro-4-(3-morpholinophenyl)pyrimidin-2-yl)amino)cyclohexane-1-carbonyl)piperidin- |
| | 4-yl)methyl)piperidin-4-yl)phenyl)amino)piperidine-2,6-dione |
| 673 | 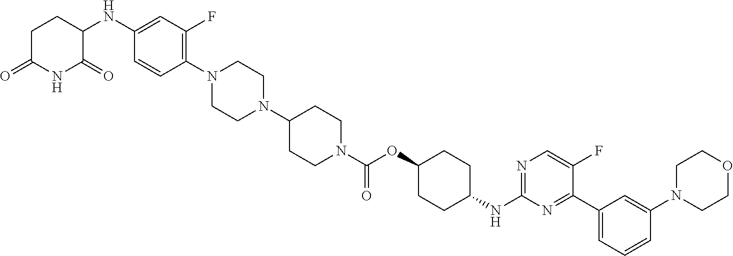 [SMILES: O=C1CCC(Nc2ccc(N3CCN(C4CCN(C(=O)O[C@H]5CC[C@H](Nc6ncc(F)c(-c7cccc(N8CCOCC8)c7)n6)CC5)CC4)CC3)c(F)c2)C(=O)N1] |
| | trans-4-((5-fluoro-4-(3-morpholinophenyl)pyrimidin-2-yl)amino)cyclohexyl 4-(4-(4-((2,6-dioxopiperidin-3-yl)amino)- |
| | 2-fluorophenyl)piperazin-1-yl)piperidine-1-carboxylate |
| 674 | 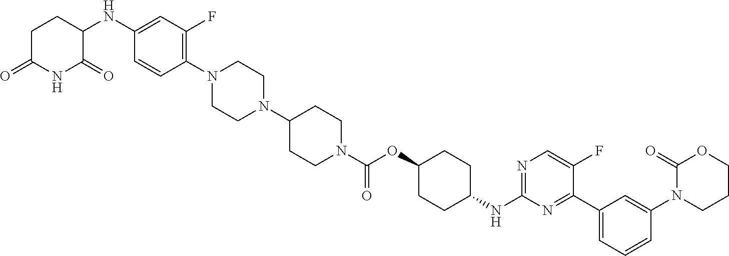 [SMILES: O=C1CCC(Nc2ccc(N3CCN(C4CCN(C(=O)O[C@H]5CC[C@H](Nc6ncc(F)c(-c7cccc(N8CCCOC8=O)c7)n6)CC5)CC4)CC3)c(F)c2)C(=O)N1] |
| | trans-4-((5-fluoro-4-(3-(2-oxo-1,3-oxazinan-3-yl)phenyl)pyrimidin-2-yl)amino)cyclohexyl 4-(4-(4-((2,6-dioxopiperidin- |
| | 3-yl)amino)-2-fluorophenyl)piperazin-1-yl)piperidine-1-carboxylate |
| 675 | 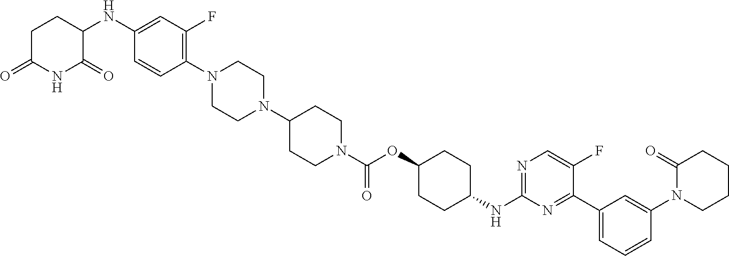 [SMILES: O=C1CCC(Nc2ccc(N3CCN(C4CCN(C(=O)O[C@H]5CC[C@H](Nc6ncc(F)c(-c7cccc(N8CCCCC8=O)c7)n6)CC5)CC4)CC3)c(F)c2)C(=O)N1] |
| | trans-4-((5-fluoro-4-(3-(2-oxopiperidin-1-yl)phenyl)pyrimidin-2-yl)amino)cyclohexyl 4-(4-(4-((2,6-dioxopiperidin- |
| | 3-yl)amino)-2-fluorophenyl)piperazin-1-yl)piperidine-1-carboxylate |
| 676 | 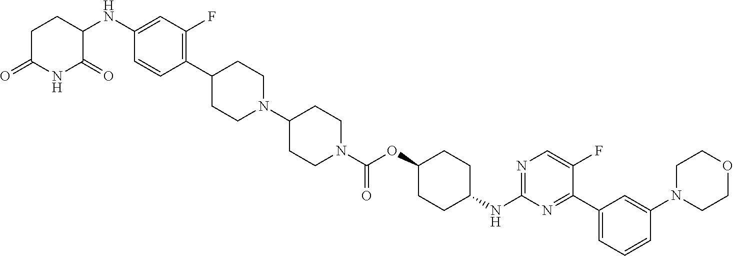 [SMILES: O=C1CCC(Nc2ccc(C3CCN(C4CCN(C(=O)O[C@H]5CC[C@H](Nc6ncc(F)c(-c7cccc(N8CCOCC8)c7)n6)CC5)CC4)CC3)c(F)c2)C(=O)N1] |
| | trans-4-((5-fluoro-4-(3-morpholinophenyl)pyrimidin-2-yl)amino)cyclohexyl 4-(4-((2,6-dioxopiperidin-3-yl)amino)- |
| | 2-fluorophenyl)-[1,4′-bipiperidine]-1′-carboxylate |
| 677 | 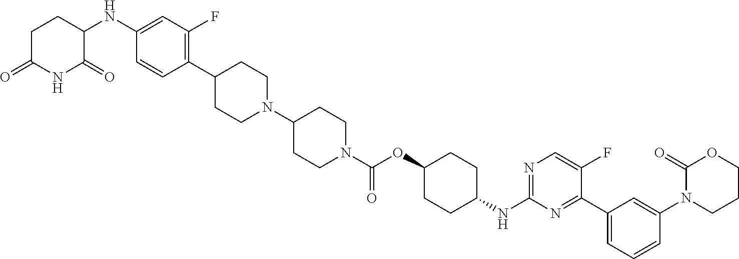 [SMILES: O=C1CCC(Nc2ccc(C3CCN(C4CCN(C(=O)O[C@H]5CC[C@H](Nc6ncc(F)c(-c7cccc(N8CCCOC8=O)c7)n6)CC5)CC4)CC3)c(F)c2)C(=O)N1] |
| | trans-4-((5-fluoro-4-(3-(2-oxo-1,3-oxazinan-3-yl)phenyl)pyrimidin-2-yl)amino)cyclohexyl 4-(4-((2,6-dioxopiperidin- |
| | 3-yl)amino)-2-fluorophenyl)-[1,4′-bipiperidine]-1′-carboxylate |
| 678 | 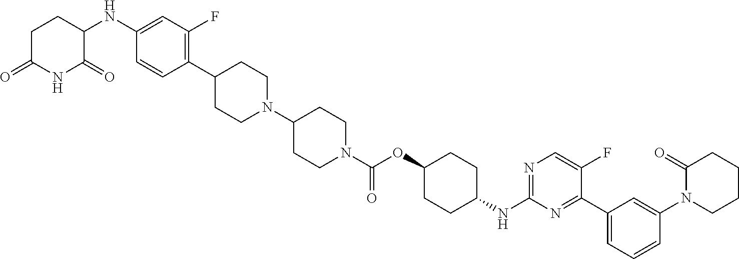 [SMILES: O=C1CCC(Nc2ccc(C3CCN(C4CCN(C(=O)O[C@H]5CC[C@H](Nc6ncc(F)c(-c7cccc(N8CCCCC8=O)c7)n6)CC5)CC4)CC3)c(F)c2)C(=O)N1] |
| | trans-4-((5-fluoro-4-(3-(2-oxopiperidin-1-yl)phenyl)pyrimidin-2-yl)amino)cyclohexyl 4-(4-((2,6-dioxopiperidin-3-yl)- |
| | amino)-2-fluorophenyl)-[1,4′-bipiperidine]-1′-carboxylate |
| 679 | 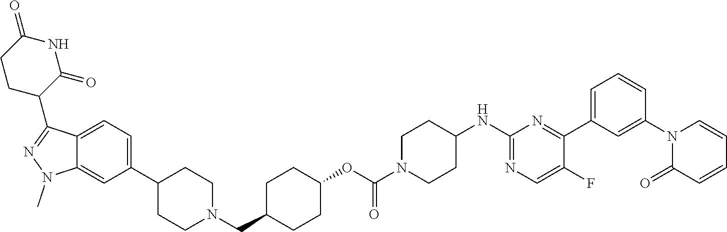 [SMILES: Cn1nc(C2CCC(=O)NC2=O)c2ccc(C3CCN(C[C@H]4CC[C@H](OC(=O)N5CCC(Nc6ncc(F)c(-c7cccc(-n8ccccc8=O)c7)n6)CC5)CC4)CC3)cc21] |
| | trans-4-((4-(3-(2,6-dioxopiperidin-3-yl)-1-methyl-1H-indazol-6-yl)piperidin-1-yl)methyl)cyclohexyl 4-((5-fluoro-4- |
| | (3-(2-oxopyridin-1(2H)-yl)phenyl)pyrimidin-2-yl)amino)piperidine-1-carboxylate |
| 680 | 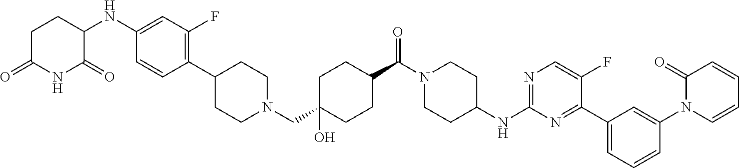 |
| | trans-3-((3-fluoro-4-(1-((4-(4-((5-fluoro-4-(3-(2-oxopyridin-1(2H)-yl)phenyl)pyrimidin-2-yl)amino)piperidine-1- |
| | carbonyl)-1-hydroxycyclohexyl)methyl)piperidin-4-yl)phenyl)amino)piperidine-2,6-dione |
| 681 | 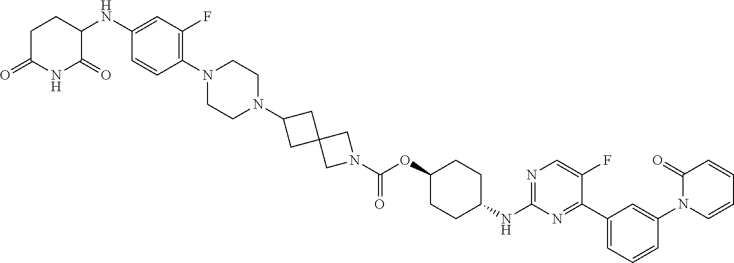 [SMILES: O=C1CCC(Nc2ccc(N3CCN(C4CC5(C4)CN(C(=O)O[C@H]4CC[C@H](Nc6ncc(F)c(-c7cccc(-n8ccccc8=O)c7)n6)CC4)C5)CC3)c(F)c2)C(=O)N1] |
| | trans-4-((5-fluoro-4-(3-(2-oxopyridin-1(2H)-yl)phenyl)pyrimidin-2-yl)amino)cyclohexyl 6-(4-(4-((2,6-dioxopiperidin- |
| | 3-yl)amino)-2-fluorophenyl)piperazin-1-yl)-2-azaspiro[3.3]heptane-2-carboxylate |
| 682 | 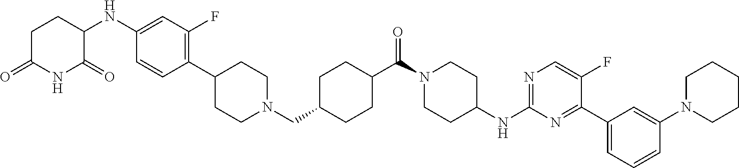 |
| | trans-3-((3-fluoro-4-(1-((4-(4-((5-fluoro-4-(3-(piperidin-1-yl)phenyl)pyrimidin-2-yl)amino)piperidine-1-carbonyl)cyclo- |
| | hexyl)methyl)piperidin-4-yl)phenyl)amino)piperidine-2,6-dione |
| 683 | 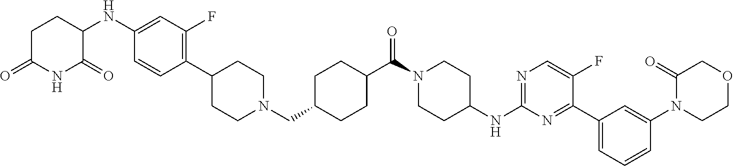 |
| | trans-3-[3-fluoro-4-[4-[[4-[4-[[5-fluoro-4-[3-(3-oxomorpholin-4-yl)phenyl]pyrimidin-2-yl]amino]cyclohexanecarbonyl] |
| | cyclohexyl]methyl]piperazin-1-yl]anilino]piperidine-2,6-dione |
| 684 | 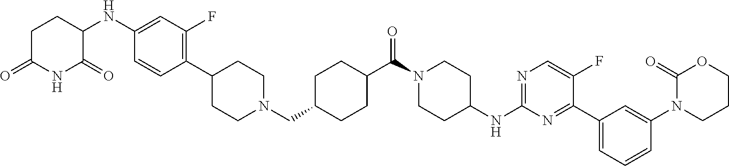 |
| | trans-3-((3-fluoro-4-(1-((4-(4-((5-fluoro-4-(3-(2-oxo-1,3-oxazinan-3-yl)phenyl)pyrimidin-2-yl)amino)piperidine-1- |
| | carbonyl)cyclohexyl)methyl)piperidin-4-yl)phenyl)amino)piperidine-2,6-dione |
| 685 | 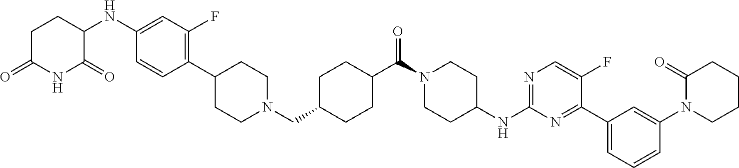 |
| | 3-((3-fluoro-4-(1-((trans-4-(4-((5-fluoro-4-(3-(2-oxopiperidin-1-yl)phenyl)pyrimidin-2-yl)amino)piperidine-1-carbonyl)- |
| | cyclohexyl)methyl)piperidin-4-yl)phenyl)amino)piperidine-2,6-dione |
| 686 | 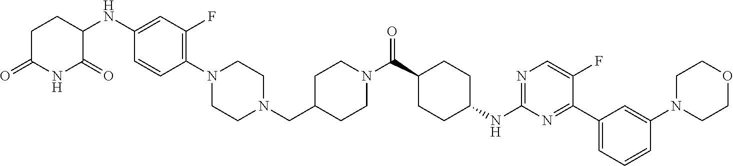 [SMILES: O=C1CCC(Nc2ccc(N3CCN(CC4CCN(C(=O)[C@H]5CC[C@H](Nc6ncc(F)c(-c7cccc(N8CCOCC8)c7)n6)CC5)CC4)CC3)c(F)c2)C(=O)N1] |
| | trans-3-((3-fluoro-4-(4-((1-(4-((5-fluoro-4-(3-morpholinophenyl)pyrimidin-2-yl)amino)cyclohexane-1-carbonyl)piperidin- |
| | 4-yl)methyl)piperazin-1-yl)phenyl)amino)piperidine-2,6-dione |
| 687 | 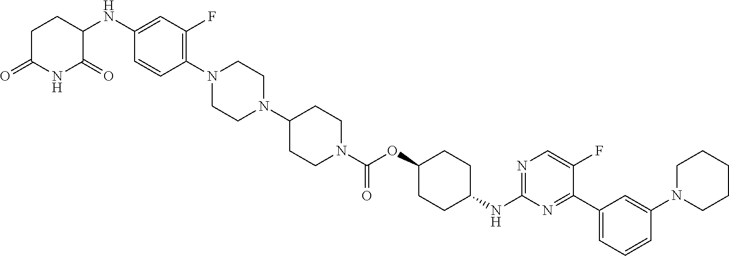 [SMILES: O=C1CCC(Nc2ccc(N3CCN(C4CCN(C(=O)O[C@H]5CC[C@H](Nc6ncc(F)c(-c7cccc(N8CCCCC8)c7)n6)CC5)CC4)CC3)c(F)c2)C(=O)N1] |
| | trans-4-((5-fluoro-4-(3-(piperidin-1-yl)phenyl)pyrimidin-2-yl)amino)cyclohexyl 4-(4-(4-((2,6-dioxopiperidin-3-yl)- |
| | amino)-2-fluorophenyl)piperazin-1-yl)piperidine-1-carboxylate |
| 688 | 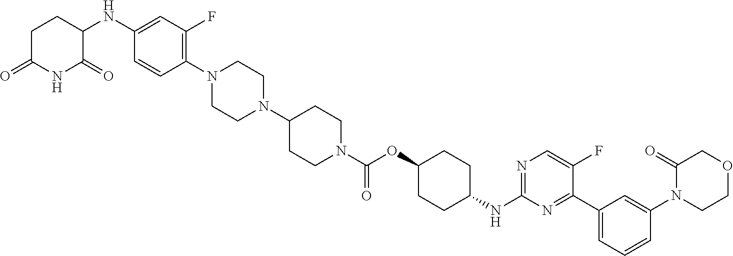 [SMILES: O=C1CCC(Nc2ccc(N3CCN(C4CCN(C(=O)O[C@H]5CC[C@H](Nc6ncc(F)c(-c7cccc(N8CCOCC8=O)c7)n6)CC5)CC4)CC3)c(F)c2)C(=O)N1] |
| | trans-4-((5-fluoro-4-(3-(3-oxomorpholino)phenyl)pyrimidin-2-yl)amino)cyclohexyl 4-(4-(4-((2,6-dioxopiperidin-3- |
| | yl)amino)-2-fluorophenyl)piperazin-1-yl)piperidine-1-carboxylate |
| 689 | 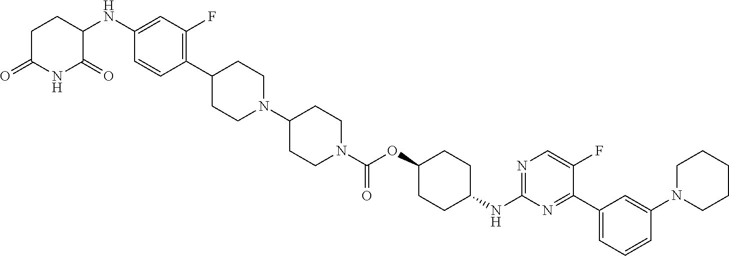 [SMILES: O=C1CCC(Nc2ccc(C3CCN(C4CCN(C(=O)O[C@H]5CC[C@H](Nc6ncc(F)c(-c7cccc(N8CCCCC8)c7)n6)CC5)CC4)CC3)c(F)c2)C(=O)N1] |
| | trans-4-((5-fluoro-4-(3-(piperidin-1-yl)phenyl)pyrimidin-2-yl)amino)cyclohexyl 4-(4-((2,6-dioxopiperidin-3-yl)amino)- |
| | 2-fluorophenyl)-[1,4′-bipiperidine]-1′-carboxylate |
| 690 | 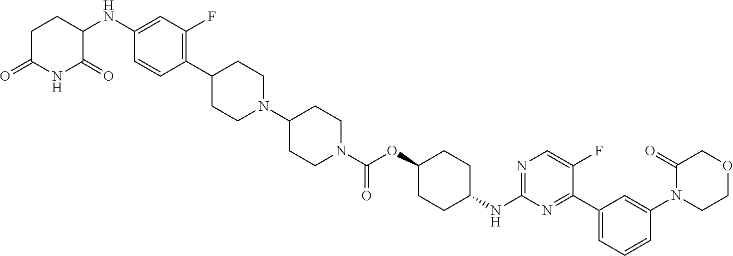 [SMILES: O=C1CCC(Nc2ccc(C3CCN(C4CCN(C(=O)O[C@H]5CC[C@H](Nc6ncc(F)c(-c7cccc(N8CCOCC8=O)c7)n6)CC5)CC4)CC3)c(F)c2)C(=O)N1] |
| | trans-4-((5-fluoro-4-(3-(3-oxomorpholino)phenyl)pyrimidin-2-yl)amino)cyclohexyl 4-(4-((2,6-dioxopiperidin-3-yl)amino)- |
| | 2-fluorophenyl)-[1,4′-bipiperidine]-1′-carboxylate |
| 691 | 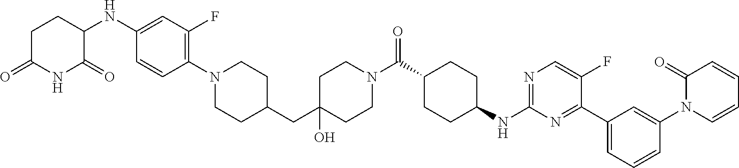 [SMILES: O=C1CCC(Nc2ccc(N3CCC(CC4(O)CCN(C(=O)[C@H]5CC[C@H](Nc6ncc(F)c(-c7cccc(-n8ccccc8=O)c7)n6)CC5)CC4)CC3)c(F)c2)C(=O)N1] |
| | trans-3-((3-fluoro-4-(4-((1-(4-((5-fluoro-4-(3-(2-oxopyridin-1(2H)-yl)phenyl)pyrimidin-2-yl)amino)cyclohexane-1- |
| | carbonyl)-4-hydroxypiperidin-4-yl)methyl)piperidin-1-yl)phenyl)amino)piperidine-2,6-dione |
| 692 | 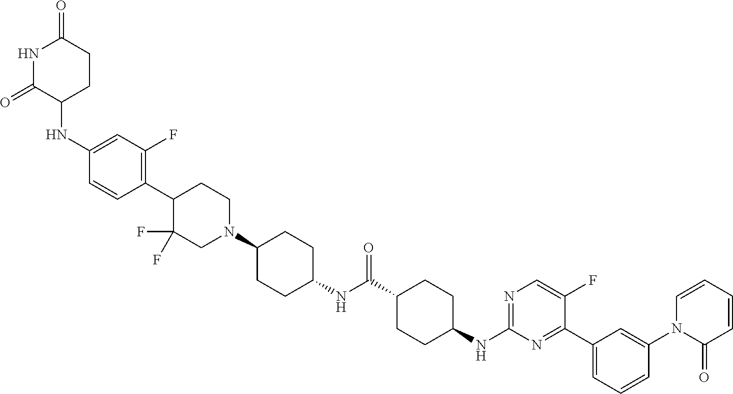 [SMILES: O=C1CCC(Nc2ccc(C3CCN([C@H]4CC[C@H](NC(=O)[C@H]5CC[C@H](Nc6ncc(F)c(-c7cccc(-n8ccccc8=O)c7)n6)CC5)CC4)CC3(F)F)c(F)c2)C(=O)N1] |
| | trans-N-(trans-4-(4-(4-((2,6-dioxopiperidin-3-yl)amino)-2-fluorophenyl)-3,3-difluoropiperidin-1-yl)cyclohexyl)-4-((5- |
| | fluoro-4-(3-(2-oxopyridin-1(2H)-yl)phenyl)pyrimidin-2-yl)amino)cyclohexane-1-carboxamide |
| 693 | 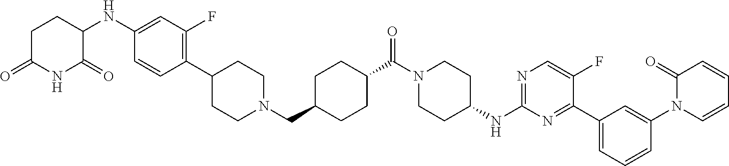 |
| | 3-((3-fluoro-4-(1-(((trans)-4-(4-((5-fluoro-4-(3-(2-oxopyridin-1(2H)-yl)phenyl)pyrimidin-2-yl)amino)piperidine-1- |
| | carbonyl)cyclohexyl)methyl)piperidin-4-yl)phenyl)amino)piperidine-2,6-dione |
| 694 | 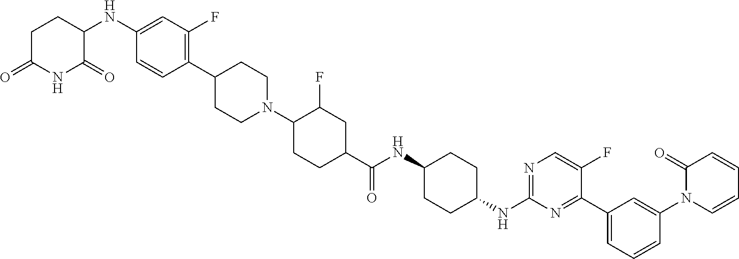 [SMILES: O=C1CCC(Nc2ccc(C3CCN(C4CCC(C(=O)N[C@H]5CC[C@H](Nc6ncc(F)c(-c7cccc(-n8ccccc8=O)c7)n6)CC5)CC4F)CC3)c(F)c2)C(=O)N1] |
| | (1R,3S,4R)-4-(4-(4-((2,6-dioxopiperidin-3-yl)amino)-2-fluorophenyl)piperidin-1-yl)-3-fluoro-N-((trans)-4-((5-fluoro- |
| | 4-(3-(2-oxopyridin-1(2H)-yl)phenyl)pyrimidin-2-yl)amino)cyclohexyl)cyclohexane-1-carboxamide |
| 695 | 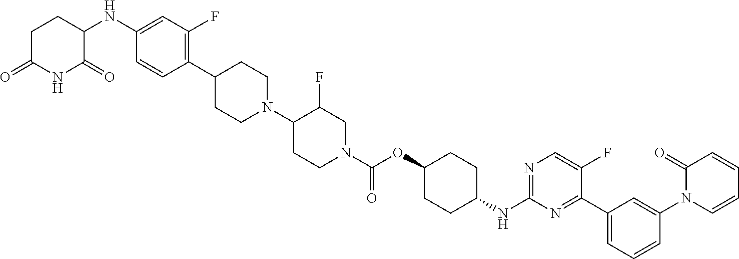 [SMILES: O=C1CCC(Nc2ccc(C3CCN(C4CCN(C(=O)O[C@H]5CC[C@H](Nc6ncc(F)c(-c7cccc(-n8ccccc8=O)c7)n6)CC5)CC4F)CC3)c(F)c2)C(=O)N1] |
| | trans-4-((5-fluoro-4-(3-(2-oxopyridin-1(2H)-yl)phenyl)pyrimidin-2-yl)amino)cyclohexyl 4-(4-((2,6-dioxopiperidin-3-yl)- |
| | amino)-2-fluorophenyl)-3′-fluoro-[1,4′-bipiperidine]-1′-carboxylate |
| 696 | 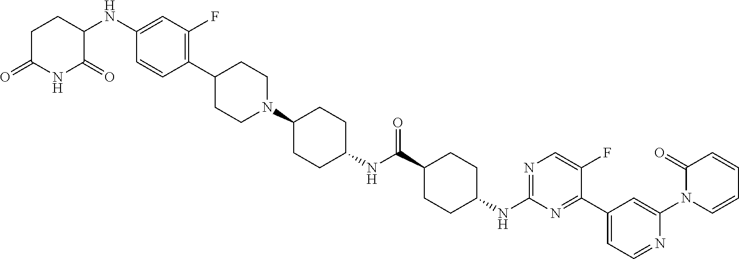 [SMILES: O=C1CCC(Nc2ccc(C3CCN([C@H]4CC[C@H](NC(=O)[C@H]5CC[C@H](Nc6ncc(F)c(-c7ccnc(-n8ccccc8=O)c7)n6)CC5)CC4)CC3)c(F)c2)C(=O)N1] |
| | trans-N-(trans-4-(4-(4-((2,6-dioxopiperidin-3-yl)amino)-2-fluorophenyl)piperidin-1-yl)cyclohexyl)-4-((5-fluoro-4-(2- |
| | oxo-2H-[1,2′-bipyridin]-4′-yl)pyrimidin-2-yl)amino)cyclohexane-1-carboxamide |
| 697 | 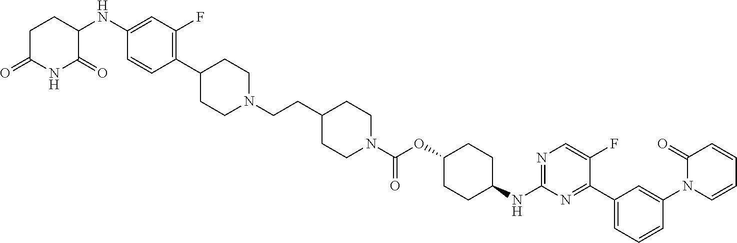 [SMILES: O=C1CCC(Nc2ccc(C3CCN(CCC4CCN(C(=O)O[C@H]5CC[C@H](Nc6ncc(F)c(-c7cccc(-n8ccccc8=O)c7)n6)CC5)CC4)CC3)c(F)c2)C(=O)N1] |
| | trans-4-((5-fluoro-4-(3-(2-oxopyridin-1(2H)-yl)phenyl)pyrimidin-2-yl)amino)cyclohexyl 4-(2-(4-(4-((2,6-dioxo- |
| | piperidin-3-yl)amino)-2-fluorophenyl)piperidin-1-yl)ethyl)piperidine-1-carboxylate |
| 698 | 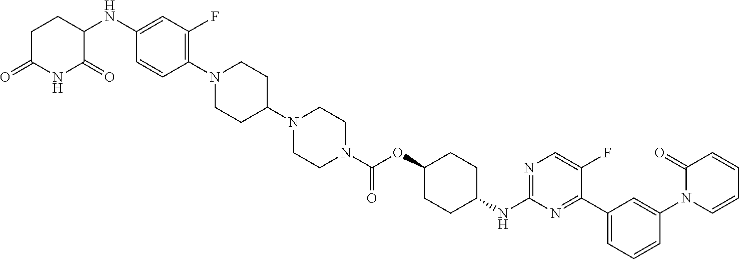 [SMILES: O=C1CCC(Nc2ccc(N3CCC(N4CCN(C(=O)O[C@H]5CC[C@H](Nc6ncc(F)c(-c7cccc(-n8ccccc8=O)c7)n6)CC5)CC4)CC3)c(F)c2)C(=O)N1] |
| | trans-4-((5-fluoro-4-(3-(2-oxopyridin-1(2H)-yl)phenyl)pyrimidin-2-yl)amino)cyclohexyl 4-(1-(4-((2,6-dioxopiperidin-3- |
| | yl)amino)-2-fluorophenyl)piperidin-4-yl)piperazine-1-carboxylate |
| 699 | 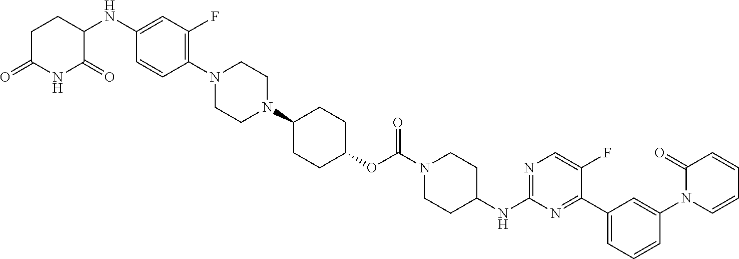 [SMILES: O=C1CCC(Nc2ccc(N3CCN([C@H]4CC[C@H](OC(=O)N5CCC(Nc6ncc(F)c(-c7cccc(-n8ccccc8=O)c7)n6)CC5)CC4)CC3)c(F)c2)C(=O)N1] |
| | trans-(1R,4r)-4-(4-(4-(((R)-2,6-dioxopiperidin-3-yl)amino)-2-fluorophenyl)piperazin-1-yl)cyclohexyl 4-((5-fluoro-4- |
| | (3-(2-oxopyridin-1(2H)-yl)phenyl)pyrimidin-2-yl)amino)piperidine-1-carboxylate |
| 700 | 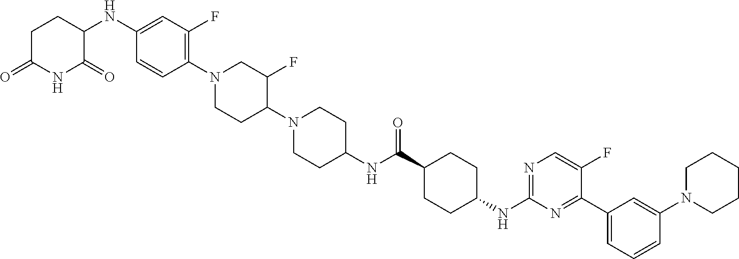 [SMILES: O=C1CCC(Nc2ccc(N3CCC(N4CCC(NC(=O)[C@H]5CC[C@H](Nc6ncc(F)c(-c7cccc(N8CCCCC8)c7)n6)CC5)CC4)C(F)C3)c(F)c2)C(=O)N1] |
| | trans-N-(1′-(4-((2,6-dioxopiperidin-3-yl)amino)-2-fluorophenyl)-3′-fluoro-[1,4′-bipiperidin]-4-yl)-4-((5-fluoro-4-(3- |
| | (piperidin-1-yl)phenyl)pyrimidin-2-yl)amino)cyclohexane-1-carboxamide |
| 701 | 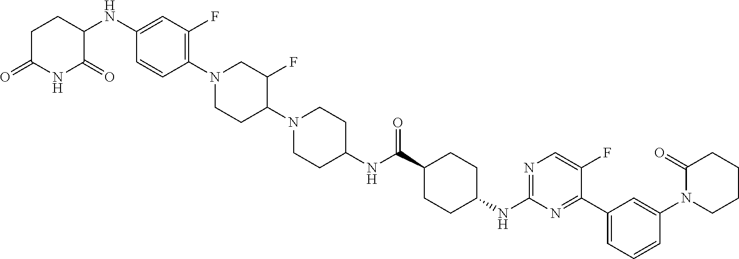 [SMILES: O=C1CCC(Nc2ccc(N3CCC(N4CCC(NC(=O)[C@H]5CC[C@H](Nc6ncc(F)c(-c7cccc(N8CCCCC8=O)c7)n6)CC5)CC4)C(F)C3)c(F)c2)C(=O)N1] |
| | trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(2- |
| | oxopiperidin-1-yl)phenyl)pyrimidin-2-yl)amino)cyclohexane-1-carboxamide |
| 702 | 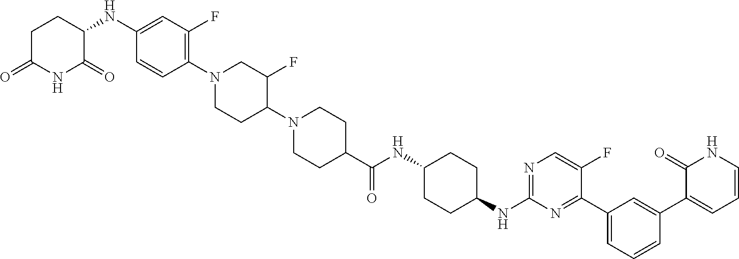 [SMILES: O=C1CC[C@H](Nc2ccc(N3CCC(N4CCC(C(=O)N[C@H]5CC[C@H](Nc6ncc(F)c(-c7cccc(-c8ccc[nH]c8=O)c7)n6)CC5)CC4)C(F)C3)c(F)c2)C(=O)N1] |
| | trans-1′-(4-(((S)-2,6-dioxopiperidin-3-yl)amino)-2-fluorophenyl)-N-((1r,4r)-4-((5-fluoro-4-(3-(2-oxo-1,2-dihydropyridin- |
| | 3-yl)phenyl)pyrimidin-2-yl)amino)cyclohexyl)-[1,4′-bipiperidine]-4-carboxamide |
| 703 | 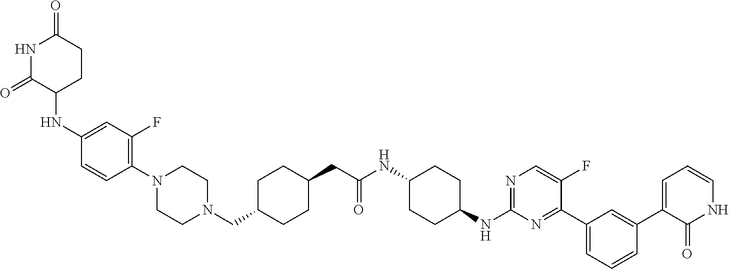 [SMILES: O=C1CCC(Nc2ccc(N3CCN(C[C@H]4CC[C@H](CC(=O)N[C@H]5CC[C@H](Nc6ncc(F)c(-c7cccc(-c8ccc[nH]c8=O)c7)n6)CC5)CC4)CC3)c(F)c2)C(=O)N1] |
| | trans-2-((1r,4R)-4-((4-(4-((2,6-dioxopiperidin-3-yl)amino)-2-fluorophenyl)piperazin-1-yl)methyl)cyclohexyl)-N- |
| | ((1r,4R)-4-((5-fluoro-4-(3-(2-oxo-1,2-dihydropyridin-3-yl)phenyl)pyrimidin-2-yl)amino)cyclohexyl)acetamide |
| 704 | 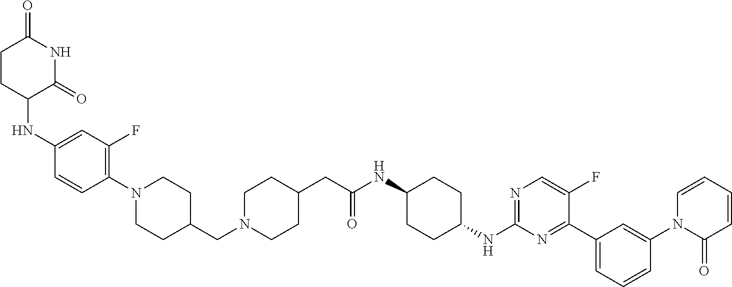 [SMILES: O=C1CCC(Nc2ccc(N3CCC(CN4CCC(CC(=O)N[C@H]5CC[C@H](Nc6ncc(F)c(-c7cccc(-n8ccccc8=O)c7)n6)CC5)CC4)CC3)c(F)c2)C(=O)N1] |
| | trans-2-(1-((1-(4-((2,6-dioxopiperidin-3-yl)amino)-2-fluorophenyl)piperidin-4-yl)methyl)piperidin-4-yl)-N-(4-((5-fluoro- |
| | 4-(3-(2-oxopyridin-1(2H)-yl)phenyl)pyrimidin-2-yl)amino)cyclohexyl)acetamide |
| 705 | 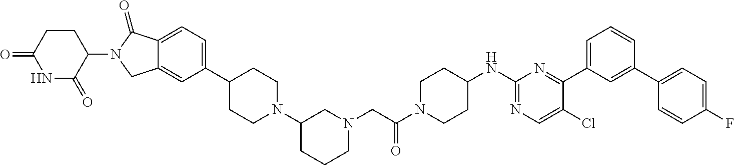 [SMILES: O=C1CCC(N2Cc3cc(C4CCN(C5CCCN(CC(=O)N6CCC(Nc7ncc(Cl)c(-c8cccc(-c9ccc(F)cc9)c8)n7)CC6)C5)CC4)ccc3C2=O)C(=O)N1] |
| | 3-(5-(1′-(2-(4-((5-chloro-4-(4′-fluoro-[1,1′-biphenyl]-3-yl)pyrimidin-2-yl)amino)piperidin-1-yl)-2-oxoethyl)-[1,3′- |
| | bipiperidin]-4-yl)-1-oxoisoindolin-2-yl)piperidine-2,6-dione |
| 706 | 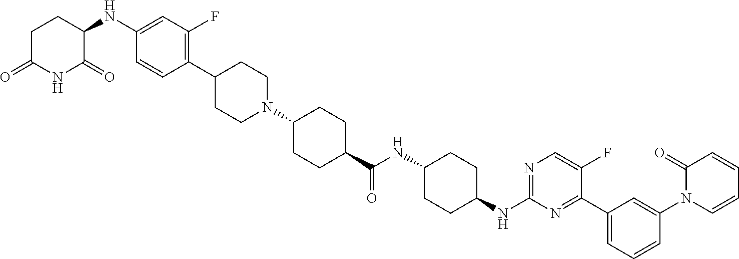 [SMILES: O=C1CC[C@@H](Nc2ccc(C3CCN([C@H]4CC[C@H](C(=O)N[C@H]5CC[C@H](Nc6ncc(F)c(-c7cccc(-n8ccccc8=O)c7)n6)CC5)CC4)CC3)c(F)c2)C(=O)N1] |
| | (1R,4R)-4-(4-(4-(((R)-2,6-dioxopiperidin-3-yl)amino)-2-fluorophenyl)piperidin-1-yl)-N-((1r,4R)-4-((5-fluoro-4-(3- |
| | (2-oxopyridin-1(2H)-yl)phenyl)pyrimidin-2-yl)amino)cyclohexyl)cyclohexane-1-carboxamide |

### Pharmaceutical Compositions

Another aspect of the present invention is directed to a pharmaceutical composition comprising a compound of Formula (I′), (I), (II), (III), (IV), or any related formula such as (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier. In some embodiments, the pharmaceutical composition comprises a therapeutically effective amount of a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and one or more pharmaceutically acceptable carriers or excipients. Representative pharmaceutically acceptable carriers and excipients are disclosed in, e.g., Remington: The Science and Practice of Pharmacy (20th ed.), ed. A. R. Gennaro, Lippincott Williams & Wilkins, 2000; Encyclopedia of Pharmaceutical Technology, eds. J. Swarbrick and J. C. Boylan, 1988-1999, Marcel Dekker, New York; and Handbook of Pharmaceutical Excipients (3^{rd} ed.) ed. A. H. Kibbe, Amer. Pharm. Assoc., 2000; each of which is incorporated herein by reference in its entirety).

Compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing may be formulated into a composition in accordance with conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York, each of which is incorporated herein by reference in its entirety). The type of formulation depends on the mode of administration which may include, for example, enteral administration, parenteral administration, and topical administration.

In some embodiments, the compounds are formulated for oral or intravenous (i.v.) administration (e.g., systemic i.v. injection). In some embodiments, the compounds are formulated for oral administration. In some embodiments, the compounds are formulated for intravenous (i.v.) administration.

Accordingly, compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing may be formulated into a solid composition, a liquid composition, a semi-solid composition, or as a gas composition.

Solid dosage forms include capsules (e.g., gelatin capsules or hard-shell capsules), tablets, pills, powders, and granules.

Liquid dosage forms for oral administration include solutions, suspensions, emulsions, micro-emulsions, and syrups.

Injectable preparations for parenteral administration may be formulated according to standard techniques, using dispersing/wetting and suspending agents. The sterile injectable preparation may be a sterile injectable solution, suspension, or emulsion in a nontoxic parenterally acceptable diluent or solvent.

The compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing may be formulated for administration by inhalation.

Compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing may be formulated for topical administration which as used herein, refers to intradermal administration to the epidermis.

In some embodiments, the topical formulations may also include an excipient, such as a penetration enhancing agent. See, e.g., Percutaneous Penetration Enhancers, Maibach H. I. and Smith H. E. (eds.), CRC Press, Inc., Boca Raton, Fla. (1995), and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997), each of which is incorporated herein by reference in its entirety.

### Dosage Amounts

The total daily dosage of the compounds thereof may be determined in accordance with standard medical practice (see, e.g., Goodman and Gilman's The Pharmacological Basis of Therapeutics, 10th Edition, A. Gilman, J. Hardman and L. Limbird, eds., McGraw-Hill Press, 155-173, 2001, which is incorporated herein by reference in its entirety).

### Methods of Use

In some aspects, provided is a method of treating a disease or disorder comprising reducing the level or activity of at least one of PIP4K2A, PIP4K2B, and PIP4K2C, comprising administering (a) a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to a subject in need thereof. In some aspects, provided is a method of treating a disease or disorder comprising reducing the level or activity of at least one of PIP4K2A, PIP4K2B, and PIP4K2C, which method comprises administration of a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, to a subject in need thereof. In some aspects, provided is a method of treating a disease or disorder comprising reducing the level or activity of at least one of PIP4K2A, PIP4K2B, and PIP4K2C, comprising administration of (a) a therapeutically effective amount of a compound of Formula (I′), (I), (II), (III), (IV), or any related formula such as Formula (I-a), (I-b), (I-c), (I-d), (I-e), (I-f), (I-g), (I-h), (I-j), (I-k), (1-1), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a therapeutically effective amount of compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to a subject in need thereof. In some aspects, provided is a method of treating a disease or disorder comprising reducing the level or activity of at least one of PIP4K2A, PIP4K2B, and PIP4K2C, which method comprises administration of a therapeutically effective amount of a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, to a subject in need thereof. In some embodiments, the methods comprise reducing the level or activity of PIP4K2C.

In some embodiments, the level or activity of at least one of PIP4K2A, PIP4K2B, and PIP4K2C in the subject is reduced by between about 5% and about 100% as compared to an untreated control subject, e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to an untreated control subject. In some embodiments, the level or activity of at least PIP4K2C is reduced.

In some embodiments, degradation or depletion of PIP4K2A, PIP4K2B, and/or PIP4K2C proteins (e.g., PIP4K2C protein) treats cancer. In some embodiments, degradation or depletion of PIP4K2A, PIP4K2B, and/or PIP4K2C proteins (e.g., PIP4K2C protein) is useful for diagnosing cancer. In some cases, the PIP4K2C protein is degraded compared to PIP4K2A and PIP4K2B proteins. In some embodiments, degradation or depletion of the PIP4K2A protein, the PIP4K2B protein, and/or the PIP4K2C protein (for instance, degradation or depletion of the PIP4K2C protein) enhances immune responses against cancer and tumors. In some embodiments, depletion or degradation of PIP4Ks, (for instance, degradation or depletion of the PIP4K2B protein) treats insulin resistance.

In some embodiments, the compounds or compositions herein reduce the level of PIP4K2B in a patient suffering from insulin resistance or suspected of suffering from insulin resistance. In some embodiments, the compounds or compositions herein decrease the level of PIP4K2C in a patient suffering from cancer, immune deficiency, autoimmune disease, or infectious disease, or a combination thereof, or suspected of suffering from cancer, immune deficiency, autoimmune disease, or infectious disease, or a combination thereof.

In some aspects, provided is a method of enhancing immune function in a subject in need thereof, comprising administering (a) a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to the subject. In some aspects, provided is a method of enhancing immune function in a subject in need thereof, comprising administering to a subject a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing. In some aspects, provided is a method of enhancing immune function in a subject in need thereof, comprising administering to a subject a therapeutically effective amount of (a) a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to the subject. In some aspects, provided is a method of enhancing immune function in a subject in need thereof, comprising administering to a subject a therapeutically effective amount of a compound of Formula (I′), (I), (II), (III), (IV), or any related formula such as Formula (I-a), (I-b), (I-c), (I-d), (I-e), (I-f), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing.

In some embodiments, immune function in the subject is enhanced by between about 5% and about 100% as compared to an untreated control subject, e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to an untreated control subject.

In some aspects, provided is a method of stimulating the immune system comprising reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering (a) a compound of Formula (I′), (I), (II), (III), (IV), or any related formula such as Formula (I-a), (I-b), (I-c), (I-d), (I-e), (If), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to the subject. In some aspects, provided is a method of stimulating the immune system comprising reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering to a subject a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing. In some embodiments, provided is a method of stimulating the immune system comprising reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering a therapeutically effective amount of (a) a compound of Formula (I′), (I), (II), (III), (IV), or any related formula such as Formula (I-a), (I-b), (I-c), (I-d), (I-e), (I-f), (I-g), (I-h), (I-J), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I′), (I), (II), (III), (IV), or any related formula such as Formula (I-a), (I-b), (I-c), (I-d), (I-e), (I—), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to the subject. In some embodiments, provided is a method of stimulating the immune system comprising reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering to a subject a therapeutically effective amount of a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing. In some embodiments, provided is a method of stimulating the immune system comprising reducing scaffolding or interaction of PIP4K2C with at least one of PIP4K2A, PIP4K2B, or phosphatidylinositol-4-phosphate 5-kinase (PIP5K) in a subject in need thereof. The reduction in the scaffolding or interaction may stimulate the immune system. In some embodiments, compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, are components of vaccines. In some embodiments, compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing are vaccine adjuvants.

In some embodiments, compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing treat a viral infection. In some embodiments, provided is a method of treating a viral infection in a subject in need thereof, comprising administering (a) a compound of Formula (I′), (I), (TT), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to the subject. In some embodiments, provided is a method of treating a viral infection in a subject in need thereof, comprising administering a compound of Formula (I′), (I), (TT), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, to the subject. In some embodiments, the compound, or a tautomer or stereoisomer thereof, or a pharmaceutically acceptable salt of any of the foregoing, is administered in combination with a vaccine for viral infections to enhance the immune response.

In some embodiments, compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing treat bacterial infections (e.g., necrotizing soft tissue infection). In some embodiments, provided is a method of treating a bacterial infection in a subject in need thereof, comprising administering (a) a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to the subject. In some embodiments, provided is a method of treating a bacterial infection in a subject in need thereof, comprising administering a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, to the subject. In some embodiments, the bacterial infection comprises necrotizing soft tissue infection.

In some embodiments, compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing treat diseases or disorders associated with at least one of PIP4K2A, PIP4K2B, and PIP4K2C. In some embodiments, provided is a method of treating a disease or disorder associated with at least one of PIP4K2A, PIP4K2B, and PIP4K2C in a subject in need thereof, comprising administering (a) a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to the subject. In some embodiments, provided is a method of treating a disease or disorder associated with at least one of PIP4K2A, PIP4K2B, and PIP4K2C in a subject in need thereof, comprising administering a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, to the subject. In some embodiments, the disease or disorder is associated with PIP4K2C. In some embodiments, compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing treat diseases or disorders associated with PIP4K2C.

In some embodiments, compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing treat cell proliferative diseases or disorders. In some embodiments, provided is a method of treating a cell proliferative disease or disorder in a subject in need thereof, comprising administering (a) a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to the subject. In some embodiments, provided is a method of treating a cell proliferative disease or disorder in a subject in need thereof, comprising administering a compound of Formula (I′), (I), (II), (III), (IV), or any related formula as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, to the subject.

Exemplary non-cancerous (e.g., cell proliferative) a diseases or disorders that may be amenable to treatment with the compounds or compositions of the present invention include inflammatory, autoimmune, neurodegenerative, heart, viral, metabolic, and genetic diseases or conditions, and chronic and acute kidney diseases or injuries.

In some embodiments, the compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing treat neurodegenerative diseases and disorders. In some embodiments, provided is a method of treating a neurodegenerative disease or disorder in a subject in need thereof, comprising administering (a) a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to the subject. In some embodiments, provided is a method of treating a neurodegenerative disease or disorder in a subject in need thereof, comprising administering a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, to the subject.

In some embodiments, the compounds of Formula (I′), (I), (II), (III), (IV), or any related formula as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing treat autoimmune diseases and disorders. In some embodiments, provided is a method of treating an autoimmune disease or disorder in a subject in need thereof, comprising administering (a) a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to the subject. In some embodiments, provided is a method of treating an autoimmune disease or disorder in a subject in need thereof, comprising administering a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, to the subject. In some embodiments, the disease or disorder is fibrosis.

In some embodiments, the compounds of Formula (I′), (I), (II), (III), (IV), or any related formula as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, are used to promote wound healing.

In other embodiments, the methods are directed to treating subjects having cancer. Compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing treat carcinomas (including primary and metastatic tumors), sarcomas, melanomas, and hematological cancers such as leukemia, lymphoma and multiple myeloma. In some embodiments, provided is a method of treating a carcinoma, sarcoma, melanoma, or hematological cancer in a subject in need thereof, comprising administering (a) a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to the subject. In some embodiments, provided is a method of treating carcinoma, sarcoma, melanoma, or hematological cancer in a subject in need thereof, comprising administering a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, to the subject. The subject may be an adult or pediatric subject. Tumors may be vascularized, not substantially vascularized, or non-vascularized. In some embodiments, tumors are solid tumors. In some embodiments, the cancer is acute myelocytic leukemia. In some embodiments, the cancer is colorectal cancer. In some embodiments, the cancer is lung cancer.

In some embodiments, provided herein is a method of degrading PIP4K2C using a compound of Formula (I′), or any related formula, or a tautomer or stereoisomer thereof, or a pharmaceutically acceptable salt of any of the foregoing. In some embodiments, provided herein is a method of degrading PIP4K2C using a compound of Formula (I′), or any related formula, or a tautomer or stereoisomer thereof, or a pharmaceutically acceptable salt of any of the foregoing, wherein Q is formula (i):

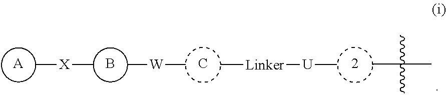

In some embodiments, provided herein is a method of degrading PIP4K2C using a compound of Formula (I′), (I), or (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing.

In some embodiments, provided herein is a method of inhibiting PIP4K using a compound of Formula (I′), or any related formula, or a tautomer or stereoisomer thereof, or a pharmaceutically acceptable salt of any of the foregoing. In some embodiments, provided herein is a method of inhibiting PIP4K using a compound of Formula (I′), or any related formula, or a tautomer or stereoisomer thereof, or a pharmaceutically acceptable salt of any of the foregoing, wherein Q is R^{Q} or Ring 5. In some embodiments, Q is R^{Q}. In some embodiments, Q is Ring 5. In some embodiments, provided herein is a method of inhibiting PIP4K using a compound of Formula (I′), (I), (II), or (III), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), or (II-g).

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodermal tumors, visual pathway and hypothalamic glioma), breast cancer (e.g., triple negative breast cancer), bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colorectal carcinoma, colorectal carcinoma with microsatellite instability (MSI), colorectal carcinoma that is microsatellite stable (MSS), colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., gastric cancer, stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma, Ann Arbor stage III and stage IV childhood Non-Hodgkin's lymphoma, ROS1-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinema, melanoma, intraocular (eye) melanoma, Merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, pancreatic ductal adenocarcinoma, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma (e.g., head and neck squamous cell carcinoma), testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

In some embodiments, the cancer is Hodgkin lymphoma or Non-Hodgkin Lymphoma, for example small lymphocytic lymphoma, Lymphoplasmacytic lymphoma, Burkitt lymphoma, Diffuse large B-cell lymphoma (DLBCL), Marginal zone Lymphoma, Follicular lymphoma, Hairy cell leukemia, and plasma cell neoplasms such as plasmacytoma and Multiple myeloma. See Hematology Reports. 2024; 16(1):164-178.

In some embodiments, the cancer is acute myeloid leukemia.

Compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, treat sarcomas including soft tissue and bone cancers, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), mesenchymous or mixed mesodermal tumor (mixed connective tissue types), and histiocytic sarcoma (immune cancer). In some embodiments, provided is a method of treating a sarcoma in a subject in need thereof, comprising administering (a) a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, or (b) a pharmaceutical composition comprising a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, and a pharmaceutically acceptable carrier, to the subject. In some embodiments, provided is a method of treating sarcoma in a subject in need thereof, comprising administering a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, to the subject.

In some embodiments, provided are methods of treating subjects having cell proliferative a disease or disorder of the hematological system, liver, brain, lung, colon, pancreas, prostate, ovary, breast, skin, or endometrium.

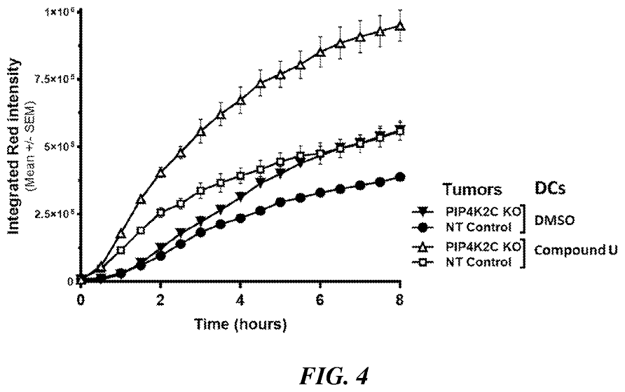

In some embodiments, provided herein are methods of treating subjects having a disease or disorder related to loss-of-function mutations of the phosphoinositide phosphatase FIG. 4. In some embodiments, the disease or disorder is selected from unis-Varón Syndrome, Charcot-Marie-Tooth, Charcot-Marie-Tooth Type 4J, polymicrogyria with epilepsy, and pediatric neurodegeneration with hypomyelination. In some embodiments, the disease or disorder is Charcot-Marie-Tooth. In some embodiments, the disease or disorder is Charcot-Marie-Tooth Type 4J. See Cao X, Lenk G M, Meisler M H. Altered phenotypes due to genetic interaction between the mouse phosphoinositide biosynthesis genes FIG. 4 and Pip4k2c. G3 (Bethesda). 2023 Aug. 9; 13(8):jkad007. doi: 10.1093/g3journal/jkad007. PMID: 36691351; PMCID: PMC10411592.

The compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing is administered to a patient as a monotherapy or by way of combination therapy. In some embodiments, the compound is administered to a patient who has received another therapy, such as chemotherapy, radioimmunotherapy, surgical therapy, immunotherapy, radiation therapy, or targeted therapy, or any combination or combinations thereof.

The methods of the present invention may entail administration of a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the compound is administered from about once a day. In other embodiments, the compound is administered twice a day (BID).

In some embodiments, use of a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, for the manufacture of a medicament is provided. In some embodiments, the use is for the manufacture of a medicament for a disease or disorder as disclosed herein.

In some embodiments, use of a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, for use in treatment is provided. In some embodiments, the use is for treatment of a disease or disorder as disclosed herein. In some embodiments, the use is for a method as disclosed herein.

In some embodiments, a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, for use in treatment is provided. In some embodiments, the compound is for use in treatment of a disease or disclosure as disclosed herein. In some embodiments, the compound is for use in a method as disclosed herein.

### Combination Therapy

The compounds of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing may be used in combination with, or concurrently with, at least one other active agent, e.g., an anti-cancer agent or regimen.

In some embodiments, the treatment may include administration of a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing, in combination with one or more additional therapeutics known for use in treating the disease or condition (e.g., cancer). Representative examples of additional active agents and treatment regimens include radiation therapy, chemotherapeutics, immunomodulators, therapeutic antibodies, and CAR-T therapy.

### Pharmaceutical Kits

The compounds and their pharmaceutically acceptable salts and stereoisomers and/or compositions comprising them may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a compound of Formula (I′), (I), (II), (III), (IV), or any related formula such as Formula (I-a), (I-b), (I-c), (I-d), (I-e), (I-f), (I-g), (I-h), (I-j), (I-k), (I-l), (I-m), (I-n), (I-o), (I-p), (I-q), (I-r), (I-s), (I-t), (I-u), (I-v), (I-w), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutical composition thereof. The kits or pharmaceutical systems of the invention may also include printed instructions for using the compounds and compositions. In one aspect, a kit comprises a compound of the disclosure or a pharmaceutically acceptable salt thereof, and a label and/or instructions for use of the compound in the treatment of a disease or disorder described herein. The kit may comprise a unit dosage form of the compound.

### ENUMERATED EMBODIMENTS

The following enumerated embodiments are representative of some aspects of the invention.

Enumerated Embodiment 1. A compound of Formula (I)

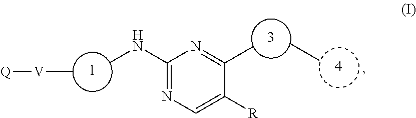

or a tautomer or stereoisomer thereof, or a pharmaceutically acceptable salt of any of the foregoing, wherein:
R is halo;
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Ring 3 is a monocyclic ring selected from the group consisting of 3- to 6-membered cycloalkyl optionally substituted with one or more R^{3a}; 4- to 6-membered heterocyclyl optionally substituted with one or more R^{3a}; phenyl optionally substituted with one or more R^{3b}; or 5- to 6-membered heteroaryl optionally substituted with one or more R^{3b};
Ring 1 is a 4- to 8-membered cycloalkyl or saturated 4- to 8-membered heterocyclyl, each of which is optionally substituted with one or more R¹;
V is a bond, —C₁₋₄alkylene-, —C(O)—, —C₁₋₄alkylene-C(O)-#, —C(O)N(R^{V})-#, —N(R^{V})C(O)-#, —C₁₋₄alkylene-C(O)N(R^{V})-# or —C₁₋₄alkylene-N(R^{V})C(O)-#, wherein # indicates the point of attachment to Ring 1; and
Q is R^{Q}, or Ring 5,
wherein Ring 5 is selected from the group consisting of 3- to 10-membered cycloalkyl optionally substituted with one or more R^{5a}, 4- to 12-membered heterocyclyl optionally substituted with one or more R^{5a}, and 5- to 12-membered heteroaryl optionally substituted with one or more R^{5b}
or
Q is of Formula (i)

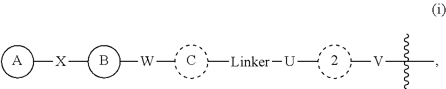

wherein:
Ring A is selected from the group consisting of

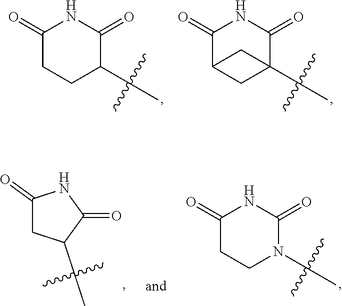 [SMILES: *C1CCC(=O)NC1=O ; *C12CC(C1)C(=O)NC2=O ; *C1CC(=O)NC1=O ; *N1CCC(=O)NC1=O]

each of which is optionally substituted with one or more C₁₋₄alkyl or halo;
X is a bond, —O—, —NH—, or —NHC(O)—;
Ring B is selected from the group consisting of

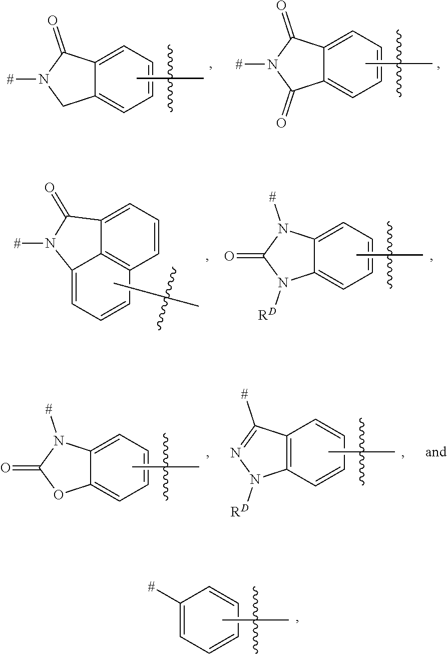

each of which is optionally substituted with one or more halo, C₁₋₄alkyl, C₁₋₄haloalkyl, or OH, and wherein R^{D} is H or C₁₋₄alkyl, wherein # indicates attachment to X;
W is a bond, —O—, —NH—, or —NHC(O)—;
Ring C is optional, and when present is 4- to 12-membered heterocyclyl optionally substituted with one or more R^{C};
   Linker is a bond, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁-6alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein
      * indicates the point of attachment to Ring C when Ring C is present, and * indicates the point of attachment to W when Ring C is absent; and
      ** indicates the point of attachment to U when Ring 2 is present, and ** indicates the point of attachment to V when Ring 2 is absent;
      L¹ is a bond or C₁₋₆ alkylene;
      L² is a 3- to 10-membered cycloalkyl or 4- to 12-membered heterocyclyl optionally substituted with one or more R^{L}; and
      L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y});
   U is absent when Ring 2 is absent, and is a bond or C(O) when Ring 2 is present;
   Ring 2 is optional, and when present is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b};
each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)₁—C₁₋₄alkyl;
each R^{2a}, R^{3a}, R^{4a}, and R^{5a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{2b}, R^{3b}, R^{4b}, and R^{5b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R^{C} is independently halo, OH, C₁₋₄alkyl, or C₁₋₄haloalkyl;
each R^{L} is independently halo, OH, C₁₋₄alkyl, or C₁₋₄haloalkyl;
each R^{Q} is independently H, halo, OH, C₁₋₄alkyl, or C₁₋₄haloalkyl;
each R^{V} is independently halo, OH, C₁₋₄alkyl, or C₁₋₄haloalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ-C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, or C₁₋₄alkylene-S(O)ₙ-C₁₋₄alkyl; and
each n is independently 0, 1, or 2;
   wherein:
(a) Ring 1 comprises an intraannular amide, or
(b) V comprises an amide, or
(c) V is taken together with an atom of Ring 1, Ring 2, or Ring 5 to which it is attached, to form an amide;
   and wherein:
when Ring 3 is 5-membered heteroaryl, Ring 4 is present and is not cycloalkyl or tetrahydropyranyl.

Enumerated Embodiment 2. The compound of Enumerated Embodiment 1, or a tautomer or stereoisomer thereof, or a pharmaceutically acceptable salt of any of the foregoing, wherein:
Ring 1 is a 4- to 8-membered cycloalkyl, saturated 4- to 8-membered heterocyclyl comprising at least one annular ether, saturated 4- to 8-membered heterocyclyl comprising at least one annular amine, or saturated 4- to 8-membered heterocyclyl comprising an annular amide, each of which is optionally substituted with one or more R¹;
and wherein:
(i) when Ring 1 is a 4- to 8-membered heterocyclyl ring comprising at least one annular amine, optionally substituted with one or more R¹, then V is attached to an annular nitrogen atom of Ring 1 and is —C(O)— or —C₁₋₄alkylene-C(O)-#, wherein # indicates the point of attachment to Ring 1;
(ii) when Ring 1 is a 4- to 8-membered heterocyclyl ring comprising at least one annular amide, optionally substituted with one or more R¹, then V is attached to an annular nitrogen atom of Ring 1 and is a bond or —C₁₋₄alkylene-; and
(iii) when Ring 1 is C₄₋₈ cycloalkyl or a 4- to 8-membered heterocyclyl ring comprising at least one annular ether and no annular amine or annular amide, each of which is optionally substituted with one or more R¹, then:
   (a) V is —C(O)N(R^{V})-#, —N(R^{V})C(O)-#, —C₁₋₄alkylene-C(O)N(R^{V})-# or —C₁₋₄alkylene-N(R^{V})C(O)-#, wherein # indicates the point of attachment to Ring 1; or
   (b) Ring 5 is 4- to 12-membered heterocyclyl optionally substituted with one or more R^{5a}, and V is —C(O)— and is attached to an annular nitrogen atom of Ring 5; or
   (c) Ring 2 is 4- to 12-membered heterocyclyl optionally substituted with one or more R^{2a}, and V is —C(O)— and is attached to an annular nitrogen atom of Ring 2.

Enumerated Embodiment 3. The compound of Enumerated Embodiment 1 or 2, wherein R is chloro.

Enumerated Embodiment 4. The compound of Enumerated Embodiment 1 or 2, wherein R is fluoro.

Enumerated Embodiment 5. The compound of any one of Enumerated Embodiments 1-4, wherein Ring 3 is 4- to 6-membered heterocyclyl optionally substituted with one or more R^{3a}.

Enumerated Embodiment 6. The compound of Enumerated Embodiment 5, wherein Ring 3 is

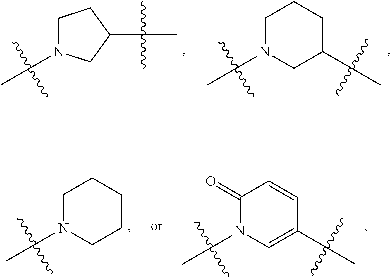 [SMILES: *C1CCN(*)C1 ; *C1CCCN(*)C1 ; *N1CCCCC1 ; *c1ccc(=O)n(*)c1]

each of which is optionally substituted with one or more R^{3a}.

Enumerated Embodiment 7. The compound of any one of Enumerated Embodiments 1-4, wherein Ring 3 is phenyl optionally substituted with one or more R^{3b}.

Enumerated Embodiment 8. The compound of any one of Enumerated Embodiments 1-4, wherein Ring 3 is 5- to 6-membered heteroaryl optionally substituted with one or more R^{3b}

Enumerated Embodiment 9. The compound of Enumerated Embodiment 8, wherein Ring 3 is

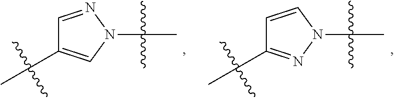 [SMILES: *c1cnn(*)c1 ; *c1ccn(*)n1]

each of which is optionally substituted with one or more R^{3b}.

Enumerated Embodiment 10. The compound of any one of Enumerated Embodiments 1-7, wherein Ring 4 is absent.

Enumerated Embodiment 11. The compound of any one of Enumerated Embodiments 1-7, wherein Ring 4 is 3- to 6-membered cycloalkyl.

Enumerated Embodiment 12. The compound of Enumerated Embodiment 11, wherein Ring 4 is cyclobutyl, cyclopentyl, or cyclohexyl, optionally substituted with one or more R^{4a}.

Enumerated Embodiment 13. The compound of any one of Enumerated Embodiments 1-9, wherein Ring 4 is 4- to 6-membered heterocyclyl optionally substituted with one or more R^{4a}.

Enumerated Embodiment 14. The compound of Enumerated Embodiment 13, wherein Ring 4 is

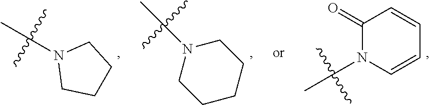 [SMILES: *N1CCCC1 ; *N1CCCCC1 ; *n1ccccc1=O]

each of which is optionally substituted with one or more R^{4a}.

Enumerated Embodiment 15. The compound of any one of Enumerated Embodiments 1-9, wherein Ring 4 is phenyl optionally substituted with one or more R^{4b}.

Enumerated Embodiment 16. The compound of any one of Enumerated Embodiments 1-9, wherein Ring 4 is 5- to 6-membered heteroaryl optionally substituted with one or more R^{4b}.

Enumerated Embodiment 17. The compound of Enumerated Embodiment 16, wherein Ring 4 is

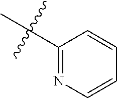 [SMILES: *c1ccccn1]

optionally substituted with one or more R^{4b}.

Enumerated Embodiment 18. The compound of any one of Enumerated Embodiments 1-4, wherein Ring 3 is phenyl optionally substituted by R^{3b}, and Ring 4 is phenyl optionally substituted by R^{4b}.

Enumerated Embodiment 19. The compound of any one of Enumerated Embodiments 1-4, wherein Ring 3 is phenyl optionally substituted by R^{3b}, or Ring 4 is phenyl optionally substituted by R^{4b}.

Enumerated Embodiment 20. The compound of any one of Enumerated Embodiments 1-4, wherein
Ring 3 is selected from the group consisting of 3- to 6-membered cycloalkyl optionally substituted with one or more R^{3a}, 4- to 6-membered heterocyclyl optionally substituted with one or more R^{3a}, and 5- to 6-membered heteroaryl optionally substituted with one or more R^{3b}; and
Ring 4 is selected from the group consisting of 3- to 6-membered cycloalkyl optionally substituted with one or more R^{4a}, 4- to 6-membered heterocyclyl optionally substituted with one or more R^{4a}, and 5- to 6-membered heteroaryl optionally substituted with one or more R^{4b}.

Enumerated Embodiment 21. The compound of any one of Enumerated Embodiments 1-9 or 13-20, wherein Ring 1 is

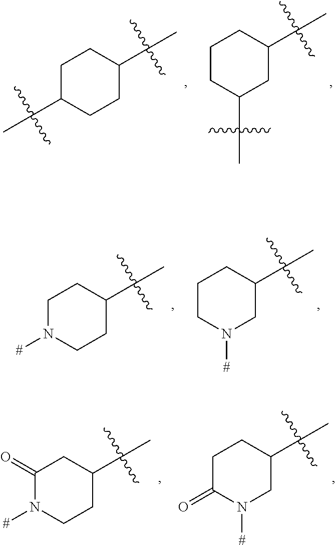

each of which is optionally substituted with one or more R¹, wherein # indicates attachment to V.

Enumerated Embodiment 22. The compound of Enumerated Embodiment 21, wherein
Ring 1 is

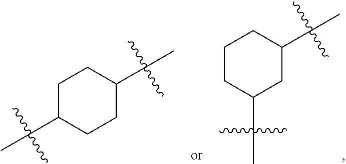 [SMILES: *C1CCC(*)CC1 ; *C1CCCC(*)C1]

each of which is optionally substituted with one or more R¹; and
V is —C(O)N(R^{V})—, —N(R^{V})C(O)—, —C₁₋₄alkylene-C(O)N(R^{V})-#, or —C₁₋₄alkylene-N(R^{V})C(O)-#, wherein ## indicates the point of attachment to Ring 1.

Enumerated Embodiment 23. The compound of Enumerated Embodiment 21, wherein
Ring 1 is

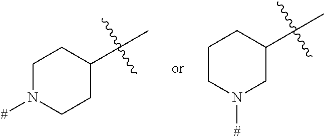

each of which is optionally substituted with one or more R¹, wherein # indicates attachment to V; and
V is —C(O)— or —C₁₋₄alkylene-C(O)-#, wherein # indicates attachment to Ring 1.

Enumerated Embodiment 23-1 The compound of Enumerated Embodiment **, wherein
Ring 1 is

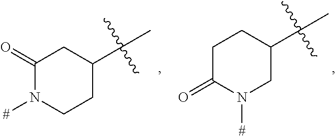

each of which is optionally substituted with one or more R¹, wherein # indicates attachment to V; and
V is bond or —C₁₋₄alkylene-.

Enumerated Embodiment 24. The compound of any one of Enumerated Embodiments 1-9 or 13-23, wherein Q is R^{Q}.

Enumerated Embodiment 25. The compound of Enumerated Embodiment 24, wherein R^{Q} is H, methyl, or acetyl.

Enumerated Embodiment 26. The compound of any one of Enumerated Embodiments 1-9 or 13-23, wherein Q is Ring 5 and the compound is of Formula (II):

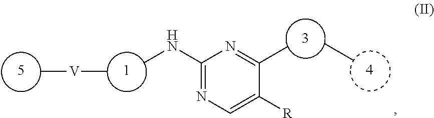

or a tautomer or stereoisomer thereof, or a pharmaceutically acceptable salt of any of the foregoing.

Enumerated Embodiment 27. The compound of any one of Enumerated Embodiments 1-9 or 13-23, wherein Q is of Formula (i) and the compound is of Formula (IV):

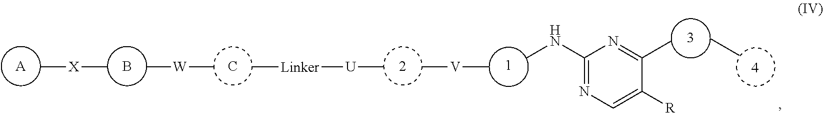

or a tautomer or stereoisomer thereof, or a pharmaceutically acceptable salt of any of the foregoing.

Enumerated Embodiment 28. The compound of Enumerated Embodiment 27, wherein Ring A is

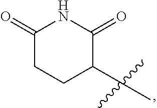 [SMILES: *C1CCC(=O)NC1=O]

optionally substituted with C₁₋₄alkyl or halo.

Enumerated Embodiment 29. The compound of Enumerated Embodiment 27 or 28, wherein X is a bond or —NH—.

Enumerated Embodiment 30. The compound of any one of Enumerated Embodiments 27-29, wherein Ring B is

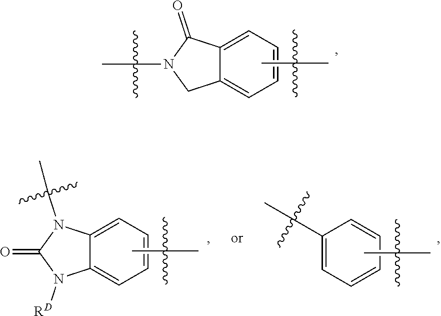

each of which is optionally substituted with one or more halo or OH, wherein R^{D} is H or C₁₋₄alkyl.

Enumerated Embodiment 31. The compound of any one of Enumerated Embodiments 27-30, wherein W is a bond or —O—.

Enumerated Embodiment 32. The compound of any one of Enumerated Embodiments 27-31, wherein Ring C is absent.

Enumerated Embodiment 33. The compound of any one of Enumerated Embodiments 27-31, wherein Ring C is

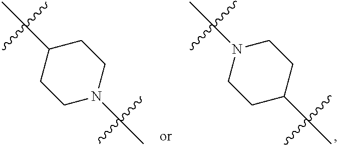 [SMILES: *C1CCN(*)CC1 ; *C1CCN(*)CC1]

each of which is optionally substituted with one or more R^{C}.

Enumerated Embodiment 34. The compound of any one of Enumerated Embodiments 27-33, wherein Linker is a bond, C₁₋₁₀alkylene, or C(O)C₁₋₁₀alkylene.

Enumerated Embodiment 35. The compound of any one of Enumerated Embodiments 27-34, wherein Ring 2 is present and U is a bond.

Enumerated Embodiment 36. The compound of any one of Enumerated Embodiments 27-35, wherein Ring 2 is 4- to 12-membered heterocyclyl optionally substituted with one or more R^{2a}.

Enumerated Embodiment 37. The compound of any one of Enumerated Embodiments 27-36, wherein Ring 2 is

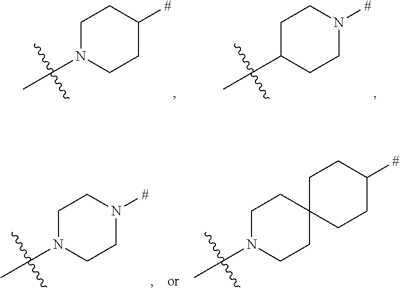

each of which is optionally substituted with one or more R², wherein # indicates attachment to V.

Enumerated Embodiment 38. The compound of any one of Enumerated Embodiments 27-36, wherein Ring 2 is

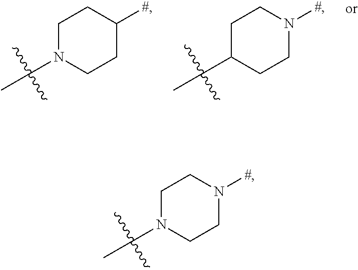

each of which is optionally substituted with one or more R^{2a}, wherein # indicates attachment to V.

Enumerated Embodiment 39. The compound of Enumerated Embodiment 38, wherein Ring 2 is

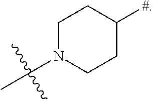

Enumerated Embodiment 40. The compound of Enumerated Embodiment 39, wherein V is —C(O)— and Ring 1 is piperidinyl.

Enumerated Embodiment 41. The compound of Enumerated Embodiment 37, wherein Ring 2 is

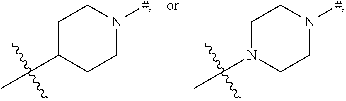

each of which is optionally substituted with one or more R^{2a}, wherein # indicates attachment to V.

Enumerated Embodiment 42. The compound of Enumerated Embodiment 41, wherein V is —C(O)— and Ring 1 is cyclohexyl.

Enumerated Embodiment 43. The compound of Enumerated Embodiment 41, wherein V is —C₁₋₄alkyl-C(O)— and Ring 1 is piperidinyl.

Enumerated Embodiment 44. The compound of any one of Enumerated Embodiments 25-33, wherein Ring 2, V, and Ring 1 are taken together to form:

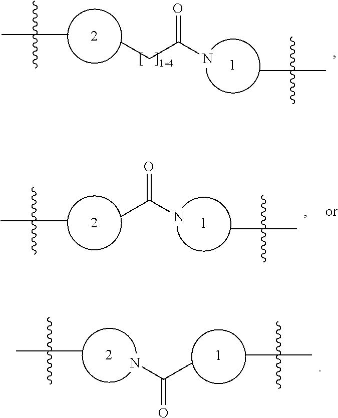

Enumerated Embodiment 45. The compound Enumerated Embodiment 44, wherein Ring 2, V, and Ring 1 are taken together to form:

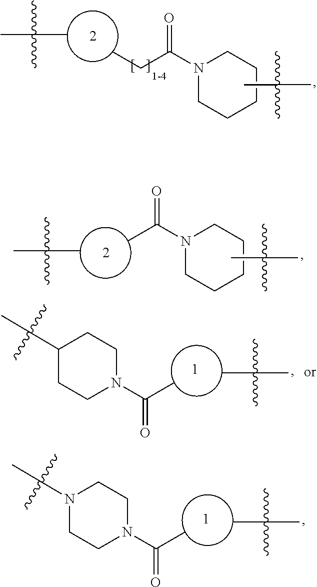

Enumerated Embodiment 46. The compound of Enumerated Embodiment 1, wherein the compound is selected from the compounds provided in Table 1 and Table 2, a tautomer or stereoisomer thereof, or a pharmaceutically acceptable salt of any of the foregoing.

Enumerated Embodiment 47. The compound of Enumerated Embodiment 46, wherein the compound is selected from the compounds provided in Table 1, a tautomer or stereoisomer thereof, or a pharmaceutically acceptable salt of any of the foregoing.

Enumerated Embodiment 48. The compound of Enumerated Embodiment 46, wherein the compound is selected from the compounds provided in Table 2, a tautomer or stereoisomer thereof, or a pharmaceutically acceptable salt of any of the foregoing.

Enumerated Embodiment 49. A pharmaceutical composition, comprising a compound of any one of Enumerated Embodiments 1-48, a tautomer or stereoisomer thereof, or a pharmaceutically acceptable salt of any of the foregoing, and a pharmaceutically acceptable excipient.

Enumerated Embodiment 50. The pharmaceutical composition of Enumerated Embodiment 49, wherein the compound is a compound of any one of Enumerated Embodiments 24-26 or 47, a tautomer or stereoisomer thereof, or a pharmaceutically acceptable salt of any of the foregoing, and a pharmaceutically acceptable excipient.

Enumerated Embodiment 51. The pharmaceutical composition of Enumerated Embodiment 49, wherein the compound is a compound of any one of Enumerated Embodiments 27-45 or 48, a tautomer or stereoisomer thereof, or a pharmaceutically acceptable salt of any of the foregoing, and a pharmaceutically acceptable excipient.

Enumerated Embodiment 52. A method of treating a disease or disorder associated with PIP4K2C, comprising administering to a subject a compound of any of Enumerated Embodiments 1-48, or the pharmaceutical composition of Enumerated Embodiment 49.

Enumerated Embodiment 53. A method of treating a disease or disorder by modulating the level or activity of PIP4K2C, comprising administering to a subject a compound of any of Enumerated Embodiments 1-48, or a tautomer or stereoisomer thereof, or a pharmaceutically acceptable salt of any of the foregoing, or the pharmaceutical composition of Enumerated Embodiment 49.

Enumerated Embodiment 54. The method of Enumerated Embodiment 53, wherein the method modulates the activity of PIP4K2C, and wherein the method comprises administering to the subject a compound of any of Enumerated Embodiments 24-26 or 47, or a tautomer or stereoisomer thereof, or a pharmaceutically acceptable salt of any of the foregoing, or the pharmaceutical composition of Enumerated Embodiment 50.

Enumerated Embodiment 55. The method of Enumerated Embodiment 53, wherein the method modulates the level of PIP4K2C, and wherein the method comprises administering to the subject a compound of any of Enumerated Embodiments 27-45 or 48, or a tautomer or stereoisomer thereof, or a pharmaceutically acceptable salt of any of the foregoing, or the pharmaceutical composition of Enumerated Embodiment 51.

Enumerated Embodiment 56. The method of any one of Enumerated Embodiments 52-55, wherein the disease or disorder is a cancer, immune deficiency, autoimmune disease, infectious disease, or a combination thereof.

Enumerated Embodiment 57. The method of Enumerated Embodiment 56, wherein the cancer is bladder cancer, breast cancer, triple negative breast cancer, cervical cancer, colorectal carcinoma, colorectal carcinoma with MSI, colorectal carcinoma with MSS, gastric cancer, head and neck squamous cell carcinoma, hepatocellular carcinoma, leukemia, lung cancer, small cell lung cancer, non-small cell lung cancer, lymphoma, melanoma, Merkel cell carcinoma, multiple myeloma, pancreatic ductal carcinoma, or renal cell carcinoma.

Enumerated Embodiment 58. The method of Enumerated Embodiment 57, wherein the cancer is breast cancer, triple negative breast cancer, colorectal carcinoma, colorectal carcinoma with MSI, colorectal carcinoma with MSS, or non-small cell lung cancer.

Enumerated Embodiment 59. The method of any one of Enumerated Embodiments 52-55, wherein the disease or disorder is a neurodegenerative disease.

Enumerated Embodiment 60. A method of treating a viral infection by modulating the level of PIP4K2C, comprising administering to a subject a compound of any of Enumerated Embodiments 27-45 or 48, or a tautomer or stereoisomer thereof, or a pharmaceutically acceptable salt of any of the foregoing, or the pharmaceutical composition of Enumerated Embodiment 51.

Enumerated Embodiment 61. A method of treating necrotizing soft tissue infection (NSTI), comprising administering to a subject a compound of any of Enumerated Embodiments 1-48, or a tautomer or stereoisomer thereof, or a pharmaceutically acceptable salt of any of the foregoing, or the pharmaceutical composition of Enumerated Embodiment 49.

Enumerated Embodiment 62. A method of modulating the level or activity of at least one of PIP4K2A, PIP4K2B, and PIP4K2C, comprising administering to a subject a compound of any of Enumerated Embodiments 1-48, or a tautomer or stereoisomer thereof, or a pharmaceutically acceptable salt of any of the foregoing, or the pharmaceutical composition of Enumerated Embodiment 49.

Enumerated Embodiment 63. A kit, comprising (i) a compound of any one of Enumerated Embodiments 1-48, or a stereoisomer or tautomer thereof, or a pharmaceutically acceptable salt of any of the foregoing, or the pharmaceutical composition of Enumerated Embodiment 49, and (ii) instructions for use in treating an PIP4K-mediated disease, disorder, or condition in an individual in need thereof.

### General Synthetic Methods and Intermediates

The compounds of the present invention can be prepared by methods known to one of ordinary skill in the art and/or by reference to the schemes shown below and the synthetic examples that follow. Exemplary synthetic routes are set forth in Schemes 1-6, and in the Examples. One of ordinary skill in the art will recognize that numerous variations in reaction conditions including variations in solvent, reagents, catalysts, reaction temperatures and times are possible for each of the reactions described. Variation of order of synthetic steps and alternative synthetic routes are also possible.

In some embodiments, provided is a method of making a compound of Formula (I′), (I), (II), (III), (IV), or any related formula such as Formula (I-a), (I-b), (I-c), (I-d), (I-e), (I-f), (I-x), (I-y), (I-z), (I-aa), (I-ab), (I-ac), (I-ad), (I-ae), (I-af), (I-ag) (II-a), (II-b), (II-c), (II-d), (II-e), (II-f), (II-g), (IV-a), (IV-b), (IV-c), (IV-d), (IV-e), (IV-f), (IV-g), (IV-h), (IV-j), (IV-k), or (IV-l), or a tautomer or stereoisomer thereof, or a pharmaceutically acceptable salt of any of the foregoing.

In some embodiments, provided is a method of preparing a compound of

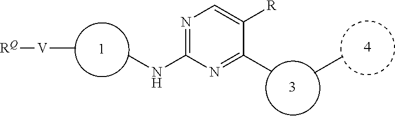

(Formula (S—III)), comprising coupling

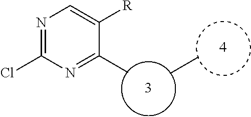

(Intermediate 1) with

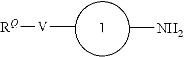

to form the compound of Formula (S—III). In some embodiments, provided is a method of preparing a compound of Formula (S—III), comprising coupling Intermediate 1 to

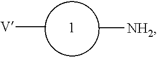

to form

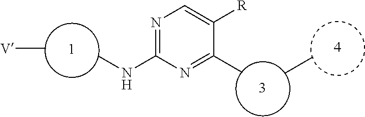

(Intermediate 2), and converting Intermediate 2 to the compound of Formula (S—III), wherein V′ is a moiety which may be converted to V—R^{Q}. For instance,

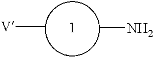

may be

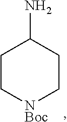

wherein V′ is a Boc protecting group, which may be deprotected later in the synthesis (i.e., V is a bond and R^{Q} is H). As another example,

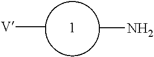

may be

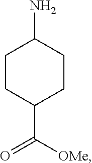

wherein V′ is a CO₂Me group, which may be converted later in the synthesis to a C(O)NH₂ (i.e., V is #—C(O)NH— wherein # indicates attachment to Ring 1, and R^{Q} is H). In some embodiments, a base is used in the coupling reaction. In some embodiments, the base is K₂CO₃. In some embodiments, the base is Cs₂CO₃. In some embodiments, the base is DIEA. In some embodiments, the coupling reaction is catalyzed. In some embodiments, the catalyst is a palladium catalyst. In some embodiments, the catalyst is Pd₂(dba)₃. In some embodiments, the catalyst is Pd(OAc)₂.

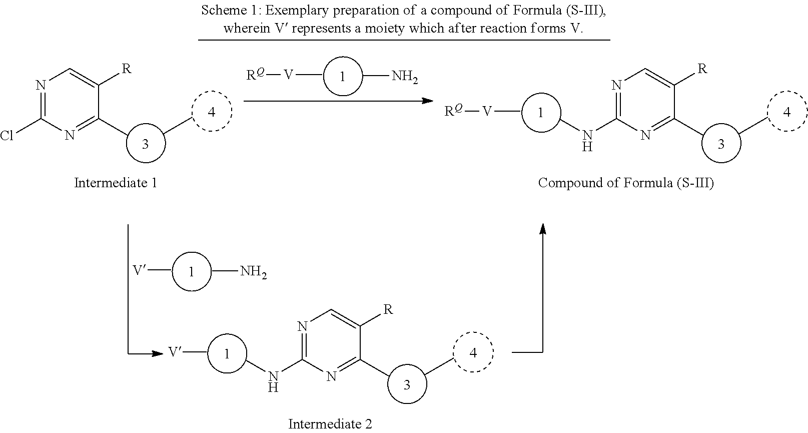

In some embodiments, provided is a method of preparing a compound of

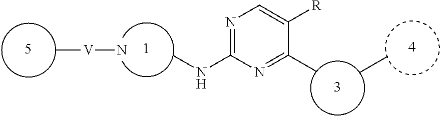

(Formula (S-II)) wherein Ring 1 is a heterocyclic ring comprising an annular amine), comprising coupling Intermediate 1 to

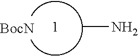

to form

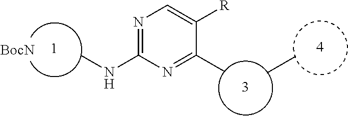

(Intermediate 3), deprotecting Intermediate 3 to form

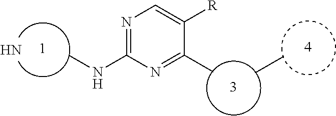

(Intermediate 4), and reacting Intermediate 4 with

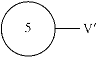

to form the compound of

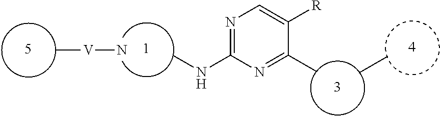

(Formula S-II)), wherein V′ represents a moiety which after reaction forms V. For example, V′ may be a —CO₂H group, which upon reaction with Intermediate 4 forms an amide (i.e., V═C(O)). In some embodiments, the coupling of Intermediate 1 to

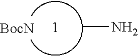

to form Intermediate 3 comprises a base. In some embodiments, the base is DIEA. In some embodiments, the deprotection of Intermediate 3 to form Intermediate 4 comprises an acid. In some embodiments, the acid is TFA. In some embodiments, the reaction of Intermediate 4 with

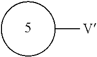

to form a compound of Formula (S-II) comprises an amide coupling reagent. In some embodiments, the amide coupling reagent is HATU.

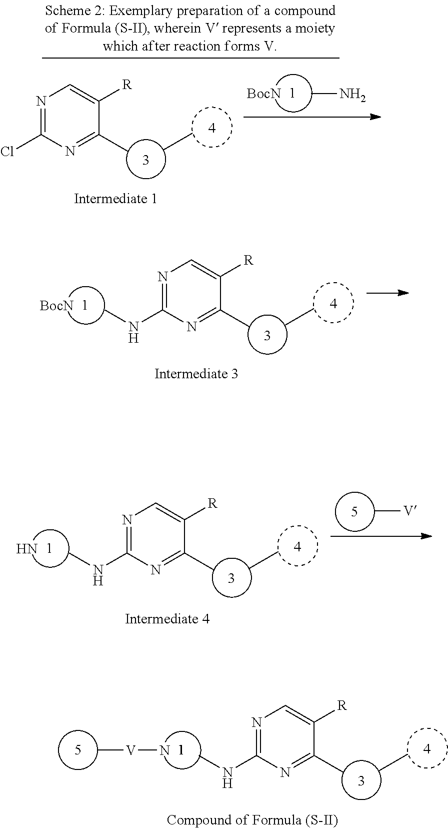

In some embodiments, provided is a method of preparing a compound of

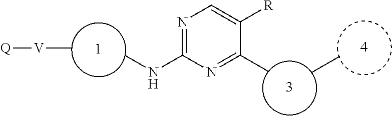

(Formula (S—IV), wherein Ring 1 is a heterocyclic ring comprising an annular amine), comprising coupling Intermediate 1 to

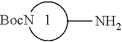

to form

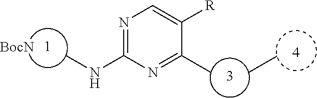

(Intermediate 3), deprotecting Intermediate 3 to form

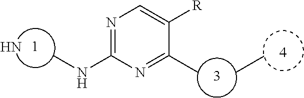

(Intermediate 4), and reacting Intermediate 4 with Q-V′ to form a compound of

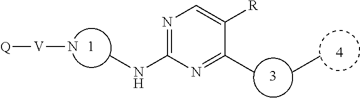

(Formula (S—IV)), wherein V′ represents a moiety which after reaction forms V. In some embodiments, provided is a method of preparing a compound of

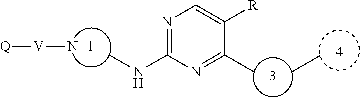

(Formula (S—IV), wherein Ring 1 is a heterocyclic ring comprising an annular amine, comprising coupling Intermediate 1 with

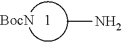

to form

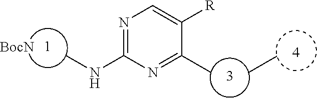

(Intermediate 3), deprotecting Intermediate 3 to form

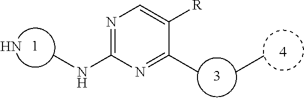

(Intermediate 4), reacting Intermediate 4 with V″ to form

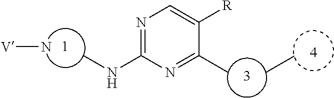

(Intermediate 2-a), and reacting Intermediate 2-a with Q′ to form a compound of

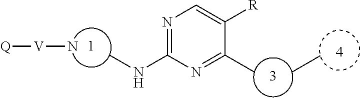

(Formula (S—IV)), wherein V″ represents a moiety which after reaction forms V′ and Q′ is a moiety which after reaction forms Q. In some embodiments, the coupling of Intermediate 1 to

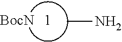

to form Intermediate 3 comprises a base. In some embodiments, the base is DIEA. In some embodiments, the deprotection of Intermediate 3 to form Intermediate 4 comprises an acid. In some embodiments, the acid is TFA. In some embodiments, the reaction of Intermediate 4 with Q-V′ to form a compound of Formula (S—IV) comprises an amide coupling reagent. In some embodiments, the amide coupling reagent is HATU. In some embodiments, wherein Ring 1 is a heterocyclic ring comprising an annular amine, the coupling of Intermediate 1 to

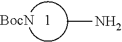

to form Intermediate 3 comprises a base. In some embodiments, the base is DIEA. In some embodiments, wherein Ring 1 is a heterocyclic ring comprising an annular amine, the coupling of Intermediate 1 to

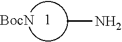

to form Intermediate 3 is catalyzed. In some embodiments, the catalyst is a palladium catalyst. In some embodiments, the catalyst is Pd(OAc)₂. In some embodiments, the deprotection of Intermediate 3 to form Intermediate 4 comprises an acid. In some embodiments, the acid is TFA. In some embodiments, the reaction of Intermediate 4 with V″ to form

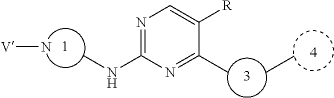

(Intermediate 2-a) comprises a base. In some embodiments, the base is TEA. In some embodiments, the reaction of Intermediate 2-a with Q′ to form a compound of Formula (S—IV) comprises a base. In some embodiments, the base is DIPEA.

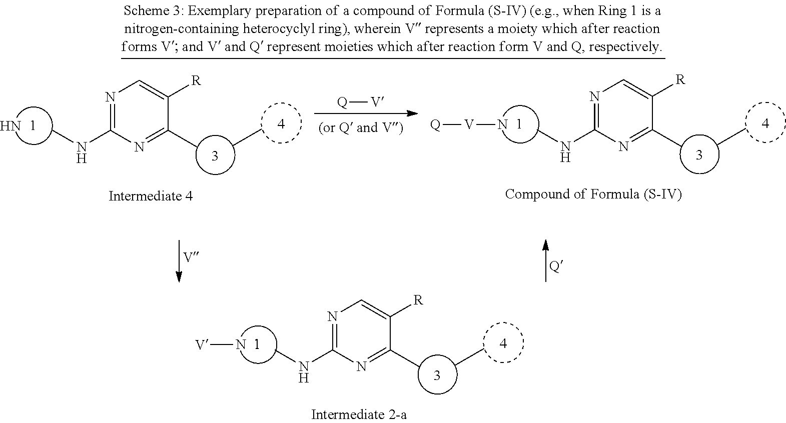

In some embodiments, provided is a method of preparing a compound of

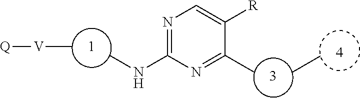

(Formula (S—IV-a)), comprising coupling Intermediate 1 to

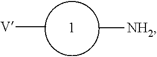

to form

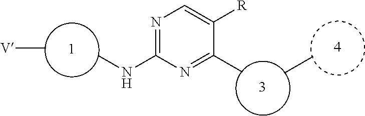

(Intermediate 2), and reacting Intermediate 2 with Q′ to form the compound of

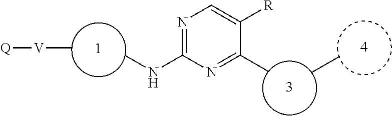

(Formula (S—IV-a)), wherein V′ represents a moiety which after reaction forms V, and Q′ represents a moiety which after reaction forms Q. In some embodiments, reacting Intermediate 2 with Q′ to form a compound of Formula (S—IV-a) comprises an amide coupling reagent. In some embodiments, the amide coupling reagent is HATU.

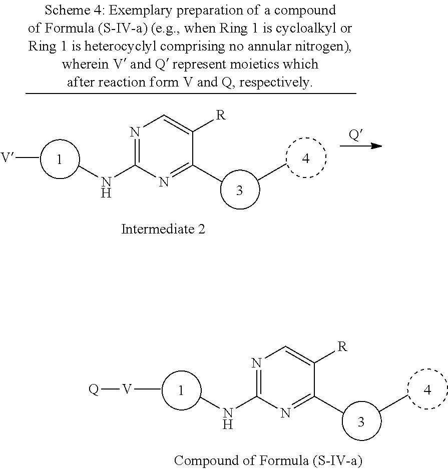

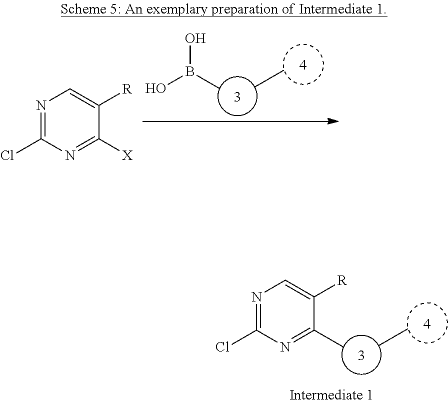

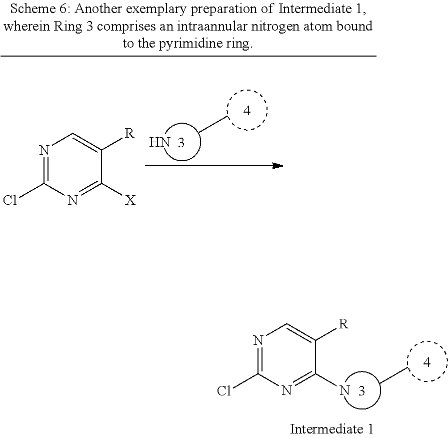

These and other aspects of the present invention will be further appreciated upon consideration of the following Examples, which are intended to illustrate certain particular embodiments of the invention but are not intended to limit its scope, as defined by the claims.

### EXAMPLES

### Synthetic Examples

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

TLC spots were visualized by UV light (254 and 220 nm). Purification by column and flash chromatography was carried out using silica gel (100-200 mesh). Solvent systems are reported as the ratio of solvents. NMR spectra were recorded on a Bruker 400 (400 MHz) spectrometer. ¹H chemical shifts are reported in δ values in ppm with tetramethylsilane (TMS, =0.00 ppm) as the internal standard. LCMS spectra were obtained on an Agilent 1200 series 6110 or 6120 mass spectrometer with ESI (+) ionization mode. LCMS spectra were obtained on a Shimadzu LC-20AD MSD:LCMS-2020 mass spectrometer with ESI (−) ionization mode See, e.g., the data provided in Tables 3 and 4.

### ABBREVIATIONS

| | |
|---|---|
| ACN, MeCN | Acetonitrile |
| AcOH | Acetic acid |
| DCM | Dichloromethane |
| DMSO | Dimethylsulfoxide |
| DIEA | Diisopropylethylamine |
| EtOAc | Ethyl acetate |
| EtOH | Ethanol |
| FA | Formic acid |
| NMP | N-methyl-2-pyrrolidone |
| MeOH | Methanol |
| PE | Petroleum ether |
| TEA | Triethylamine |
| TFA | Trifluoroacetic acid |
| THF | Tetrahydrofuran |

### Example 1. Preparation Examples

### Synthesis of 4-([1,1′-biphenyl]-3-yl)-2,5-dichloropyrimidine (Compound A)

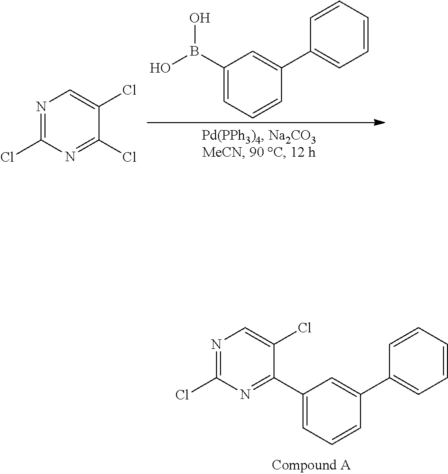

Synthesis of 4-([1,1′-biphenyl]-3-yl)-2,5-dichloropyrimidine (Compound A). To a solution of (3-phenylphenyl)boronic acid (100 g, 505 mmol, 1.00 eq) and 2,4,5-trichloropyrimidine (92.6 g, 505 mmol, 1.00 eq) in MeCN (1500 mL) were added Na₂CO₃ (107 g, 1.01 μmol, 2.00 eq) and Pd(PPh₃)₄ (5.84 g, 5.05 mmol, 0.0100 eq) at 25 °C. The resulting mixture was stirred at 90 °C for 12 h under N₂. LCMS showed the desired mass was detected. The mixture was filtered and the filtrate was concentrated under reduced pressure to give a residue. The crude product was triturated with MeOH (2 L) and H₂O (4 L) at 15 °C for 0.5 h and then filtered. The filter cake was dried under reduced pressure to give the title compound (95.0 g, 315 mmol, 31.2% yield) as a white solid. LCMS (ES+): m/z 301.0 [M+H]⁺.

### Synthesis of 4-([1,1′-biphenyl]-3-yl)-5-chloro-N-(piperidin-4-yl)pyrimidin-2-amine (Compound B)

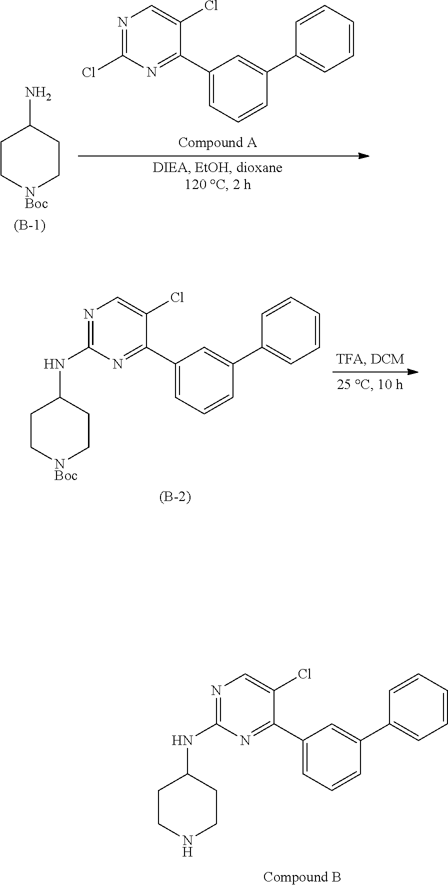

Synthesis of tert-butyl 4-((4-([1,1′-biphenyl]-3-yl)-5-chloropyrimidin-2-yl)amino)piperidine-1-carboxylate (B-2). The solution of tert-butyl 4-aminopiperidine-1-carboxylate (B-1) (366 mg, 1.83 mmol, 1.10 eq), 2,5-dichloro-4-(3-phenylphenyl)pyrimidine (Compound A) (500 mg, 1.66 mmol, 1.00 eq) and DIEA (644 mg, 4.98 mmol, 868 μL, 3.00 eq) in EtOH (5 mL) and dioxane (5 mL) was stirred at 120 °C for 2 h. LCMS showed the desired MS was detected. The reaction mixture was concentrated. The residue was purified by column chromatography (SiO₂, Petroleum ether:Ethyl acetate=10:1 to 1:1) to give the title compound (B-2) (390 mg, 839 μmol, 50.5% yield) as a yellow solid. LCMS (ES−): m/z 463.2.

Synthesis of 4-([1,1′-biphenyl]-3-yl)-5-chloro-N-(piperidin-4-yl)pyrimidin-2-amine (Compound B). To a solution of tert-butyl 4-((4-([1,1′-biphenyl]-3-yl)-5-chloropyrimidin-2-yl)amino)piperidine-1-carboxylate (B-2) (389 mg, 837 μmol, 1.00 eq) in DCM (5 mL) was added TFA (95.4 mg, 837 μmol, 62.0 μL, 1.00 eq). The mixture was stirred at 25 °C for 10 h. LCMS showed the desired MS was detected. The reaction mixture was concentrated. The residue was purified by prep-HPLC (column: Phenomenex Luna C18 75*30 mm*3 μm; mobile phase: [water (FA)-ACN]; B %: 20%-60%, 8 min) to give Compound B (79.1 mg, 175 μmol, 17.5% yield, 97.4% purity) as a white solid. LCMS (ES+): m/z 365.0 [M+H]⁺. ¹H NMR (400 MHz, DMSO-d₆) δ 8.46 (s, 1H), 8.00 (br s, 1H), 7.81 (br d, J=7.6 Hz, 1H), 7.75 (br s, 1H), 7.70 (br d, J=7.4 Hz, 2H), 7.65-7.58 (m, 1H), 7.50 (t, J=7.6 Hz, 2H), 7.44-7.37 (m, 1H), 3.95 (br d, J=6.3 Hz, 2H), 3.22 (br d, J=11.5 Hz, 2H), 2.89 (br t, J=10.5 Hz, 2H), 2.01 (br d, J=11.4 Hz, 2H), 1.64 (br s, 2H).

### Synthesis of (S)-1-(3-aminopiperidin-1-yl)ethan-1-one (Compound C)

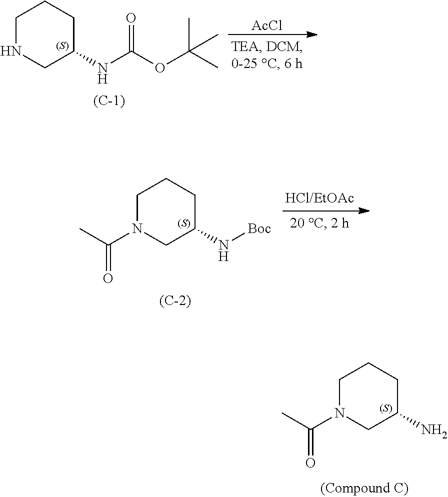

Synthesis of tert-butyl (S)-(1-acetylpiperidin-3-yl)carbamate (C-2). To a solution of tert-butyl (S)-piperidin-3-ylcarbamate (C-1) (5 g, 24.97 mmol, 1 eq) and TEA (3.79 g, 37.45 mmol, 5.21 mL, 1.5 eq) in DCM (50 mL) was slowly added acetyl chloride (1.76 g, 22.47 mmol, 1.60 mL, 0.9 eq) at 0 °C. The reaction mixture was stirred at 25 °C for 6 h. LCMS showed tert-butyl (S)-piperidin-3-ylcarbamate (C-1) was consumed completely and desired mass was detected. Then diluted with DCM (200 mL), washed with saturated NaHCO₃ (50 ml) and brine (100 ml), dried over Na₂SO₄ and filtered, concentrated under reduced pressure to give title compound (C-2) (5.67 g, 23.39 mmol, 93.69% yield) as a white solid. LCMS (ES⁺): m/z 187.1 [M+H−56]⁺.

Synthesis of (S)-1-(3-aminopiperidin-1-yl)ethan-1-one (Compound C). A mixture of tert-butyl (S)-(1-acetylpiperidin-3-yl)carbamate (C-2) (200 mg, 825.38 μmol, 1 eq) in HCl/EtOAc (5 mL) was stirred at 20 °C for 2 h. LCMS showed tert-butyl (S)-(1-acetylpiperidin-3-yl)carbamate (C-2) was consumed completely and desired mass was detected. The reaction mixture was concentrated to give title compound (C) (110 mg, 615.70 mol, 74.60% yield, HCl) as a white solid, which was used in next step directly. LCMS (ES⁺): m/z 143.1 [M+H]⁺.

### Synthesis of cis-N-((1s,4s)-4-aminocyclohexyl)acetamide (Compound D)

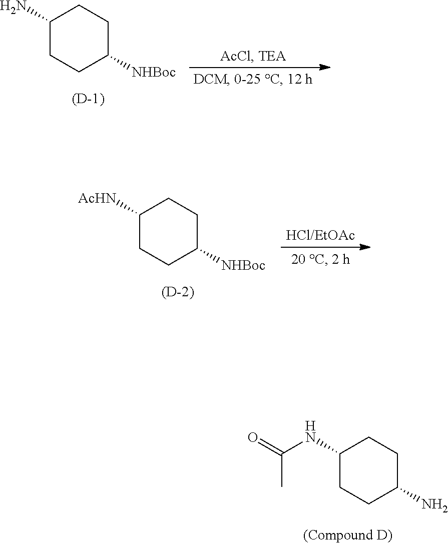

Synthesis of cis-tert-butyl ((1s,4s)-4-acetamidocyclohexyl)carbamate (C-2) To a solution of cis-tert-butyl ((1s,4s)-4-aminocyclohexyl)carbamate (D-1) (4.5 g, 21.00 mmol, 1 eq) in DCM (40 mL) were added TEA (3.19 g, 31.50 mmol, 4.38 mL, 1.5 eq), and then added acetyl chloride (1.48 g, 18.90 mmol, 1.34 mL, 0.9 eq) in DCM (10 mL) at 0 °C, and the reaction mixture was stirred at 25 °C for 12 h under N₂. LCMS showed cis-tert-butyl ((1s,4s)-4-aminocyclohexyl)carbamate (D-1) was consumed completely and the desired mass was detected. The reaction mixture was diluted with H₂O (50 mL) and extracted with DCM (50 mL*3). The combined organic layers were washed with brine (50 mL), and then dried over Na₂SO₄, filtered and the filtrate was concentrated to give title compound (D-2) (5 g, 19.51 mmol, 92.89% yield) as a yellow solid which was used in next step directly. LCMS (ES⁺): m/z 201.3 [M+H−56]⁺.

Synthesis of cis-N-((1s,4s)-4-aminocyclohexyl)acetamide (Compound D) A mixture of cis-tert-butyl ((1s,4s)-4-acetamidocyclohexyl)carbamate (D-2) (200 mg, 780.21 μmol, 1 eq) in HCl/EtOAc (5 mL) stirred at 20 °C for 2 h. LCMS showed cis-tert-butyl ((1s,4s)-4-acetamidocyclohexyl)carbamate (D-2) was consumed completely and desired mass was detected. The reaction mixture was concentrated to give title compound (Compound D) (120 mg, 768.12 μmol, 98.45% yield) as a white solid, which was used in next step directly. LCMS (ES⁺): m/z 157.1 [M+H]⁺.

### Synthesis of trans-N-((1r,4r)-4-aminocyclohexyl)acetamide (Compound E)

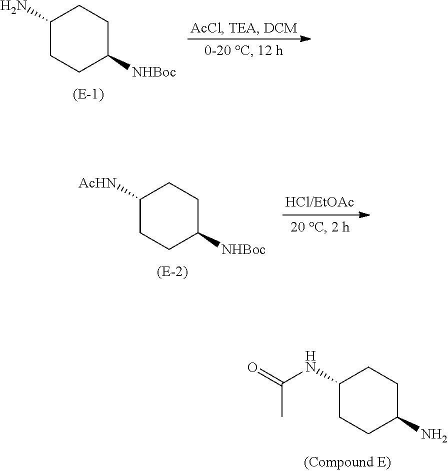

Synthesis of trans-tert-butyl ((1r,4r)-4-acetamidocyclohexyl)carbamate (E-2). To a solution of trans-tert-butyl ((1r,4r)-4-aminocyclohexyl)carbamate (E-1) (5.00 g, 19.94 mmol, 1.00 eq, HCl) and TEA (3.03 g, 29.91 mmol, 4.16 mL, 1.5 eq) in DCM (50 mL) was added acetyl chloride (1.41 g, 17.95 mmol, 1.28 mL, 0.9 eq) in DCM (20 mL) at 0 °C and the mixture was stirred at 20 °C for 12 h. LCMS showed trans-tert-butyl ((1r,4r)-4-aminocyclohexyl)carbamate (E-1) was consumed completely and the desired mass was detected. The reaction was quenched by sat.aq.NaHCO₃ (40 mL) and extracted with EtOAc (40 mL*3). The combined organic layers were washed with brine (50 mL), dried over Na₂SO₄ and concentrated under reduced pressure to give title compound (E-2) (5.1 g, 19.90 mmol, 99.78% yield) as a white solid. LCMS (ES⁺): m/z 201.2 [M+H−56]⁺.

Synthesis of trans-N-((1r,4r)-4-aminocyclohexyl)acetamide (Compound E). A mixture of trans-tert-butyl ((1r,4r)-4-acetamidocyclohexyl)carbamate (E-2) (200 mg, 780.21 μmol, 1 eq) in HCl/EtOAc (4 M, 3.33 mL, 17.09 eq) was stirred at 20 °C for 2 hr. LCMS showed trans-tert-butyl ((1r,4r)-4-acetamidocyclohexyl)carbamate (E-2) was consumed completely and desired mass was detected. The reaction was concentrated to give title compound (Compound E) (120 mg, crude, HCl) as a white solid. LCMS (ES⁺): m/z 157.1 [M+H]⁺.

Synthesis of trans-(1r,4r)-4-amino-N-methylcyclohexane-1-carboxamide (Compound F)

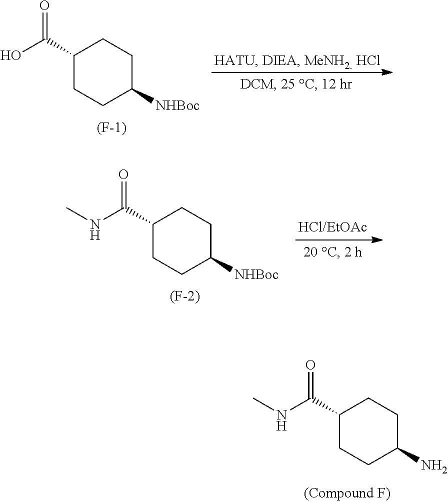

Synthesis of trans-tert-butyl ((1r, 4r)-4-(methyl carbamoyl) cyclohexyl) carbamate (F-2). To a solution of trans-(1r,4r)-4-((tert-butoxycarbonyl)amino)cyclohexane-1-carboxylic acid (F-1) (3 g, 12.33 mmol, 1 eq) in DCM (5 mL) was added methanamine; hydrochloride (1.25 g, 18.50 mmol, 1.5 eq), DIEA (4.78 g, 36.99 mmol, 6.44 mL, 3 eq) and HATU (4.69 g, 12.33 mmol, 1 eq) at 25 °C, the mixture was stirred at 25 °C for 12 h. LCMS showed trans-(1r,4r)-4-((tert-butoxycarbonyl)amino)cyclohexane-1-carboxylic acid (F-1) was remaining but the desired mass was detected. The reaction liquid is concentrated directly under vacuum to obtain the residue. The crude product was rinsed with MTBE (50 mL) and then concentrated under vacuum to give title compound (F-2) (2.2 g, 8.58 mmol, 69.60% yield) as a white solid. LCMS (ES+): m/z 201.1 [M+H−56]⁺.

Synthesis of trans-(1r,4r)-4-amino-N-methylcyclohexane-1-carboxamide (Compound F). A mixture of trans-tert-butyl ((1r, 4r)-4-(methyl carbamoyl) cyclohexyl) carbamate (F-2) (200 mg, 780.21 μmol, 1 eq) in HCl/EtOAc (4 M, 3.33 mL, 17.09 eq) was stirred at 20 °C for 2 h. LCMS showed trans-tert-butyl ((1s, 4s)-4-(methyl carbamoyl) cyclohexyl) carbamate (F-2) was consumed completely and desired mass was detected. The reaction was concentrated to give title compound (Compound F) (120 mg, crude, HCl) as a white solid. LCMS (ES⁺): m/z 157.1 [M+H]⁺.

### Synthesis of cis-(1s,4s)-4-amino-N-methylcyclohexane-1-carboxamide (Compound G)

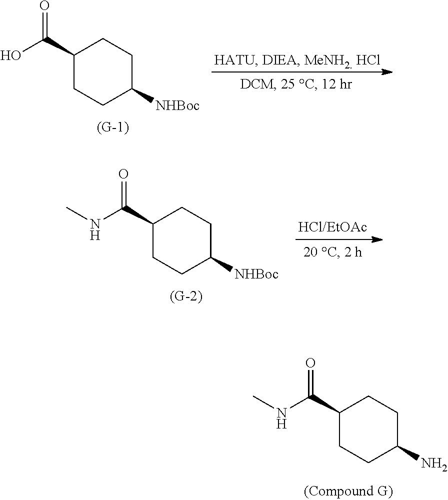

Synthesis of cis-tert-butyl ((1s,4s)-4-(methylcarbamoyl)cyclohexyl)carbamate (G-2). To a solution of cis-(1s,4s)-4-((tert-butoxycarbonyl)amino)cyclohexane-1-carboxylic acid (G-1) (5 g, 20.55 mmol, 1 eq) in DCM (100 mL) was added HATU (8.60 g, 22.61 mmol, 1.1 eq), DIEA (7.97 g, 61.65 mmol, 10.74 mL, 3 eq), and methanamine; hydrochloride (1.67 g, 24.66 mmol, 1.2 eq). The reaction stirred at 25 °C for 2 h. LCMS showed cis-(1s,4s)-4-((tert-butoxycarbonyl)amino)cyclohexane-1-carboxylic acid (G-1) was consumed completely and the desired mass was detected. The reaction mixture was added to H₂O (20 mL), and extracted with DCM (30 mL*3). The combined organic layers were washed with brine (20 mL*3), dried over Na₂SO₄, filtered and the filtrate was concentrated under reduced pressure to give a residue. The resulting solid was collected by filtration, washed with EtOAc/MTBE=1:3 (60 mL) and dried to give title compound (G-2) (4.74 g, 18.31 mmol, 89.08% yield, 99% purity) as a white solid. LCMS (ES+): m/z 201.1 [M+H-56]⁺.

Synthesis of cis-(1s,4s)-4-amino-N-methylcyclohexane-1-carboxamide (Compound G). A mixture of cis-tert-butyl ((1s, 4s)-4-(methyl carbamoyl) cyclohexyl) carbamate (G-2) (200 mg, 780.21 μmol, 1 eq) in HCl/EtOAc (4 M, 3.33 mL, 17.09 eq) was stirred at 20 °C for 2 h. LCMS showed cis-tert-butyl ((1s, 4s)-4-(methyl carbamoyl) cyclohexyl) carbamate (G-2) was consumed completely and desired mass was detected. The reaction was concentrated to give title compound (Compound G) (120 mg, crude, HCl) as a white solid. LCMS (ES⁺): m/z 157.2 [M+H]⁺.

Synthesis of cis-3-amino-N-methylcyclobutane-1-carboxamide (Compound H)

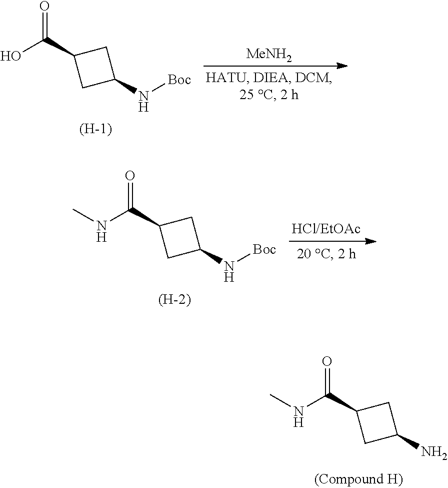

Synthesis of cis-tert-butyl (3-(methylcarbamoyl)cyclobutyl)carbamate. (H-2). To a solution of cis-3-((tert-butoxycarbonyl)amino)cyclobutane-1-carboxylic acid (H-1) (5 g, 23.23 mmol, 1 eq) in DCM (70 mL) was added HATU (9.72 g, 25.55 mmol, 1.1 eq), DIEA (9.01 g, 69.69 mmol, 12.14 mL, 3 eq) and MeNH₂ (1.73 g, 25.55 mmol, 1.1 eq, HCl). The mixture was stirred at 25 °C for 2 hr. LCMS showed cis-3-((tert-butoxycarbonyl)amino)cyclobutane-1-carboxylic acid (H-1) was consumed completely and desired mass was detected. The reaction mixture was concentrated to give a residue. The residue was diluted with H₂O (50 mL) and extracted with EtOAc (50 mL*3). The combined organic layers were dried over Na₂SO₄, filtered and the filtrate was concentrated to give a residue. The residue was triturated with Petroleum ether (20 mL*2). The resulting solid was collected by filtration, washed with Petroleum ether (10 mL) and dried to give title compound (H-2) (4.1 g, 17.96 mmol, 77.32% yield) as a white solid. LCMS (ES+): m/z 173.1 [M+H−56]⁺.

Synthesis of cis-3-amino-N-methylcyclobutane-1-carboxamide (Compound H). A mixture of cis-tert-butyl (3-(methylcarbamoyl)cyclobutyl)carbamate (H-2) (100 mg, 438.04 μmol, 1 eq) in HCl/EtOAc (4 M, 2.14 mL, 19.57 eq) was stirred at 20 °C for 2 hr. LCMS showed cis-tert-butyl (3-(methylcarbamoyl)cyclobutyl)carbamate (H-2) was consumed completely and the desired mass was detected. Vacuum distillation removes solvents to give title compound (Compound H) (72 mg, crude, HCl) as a white solid. LCMS (ES+): m/z 129.2 [M+H]⁺.

### Synthesis of trans-(1r,3r)-3-amino-N-methylcyclobutane-1-carboxamide (Compound J)

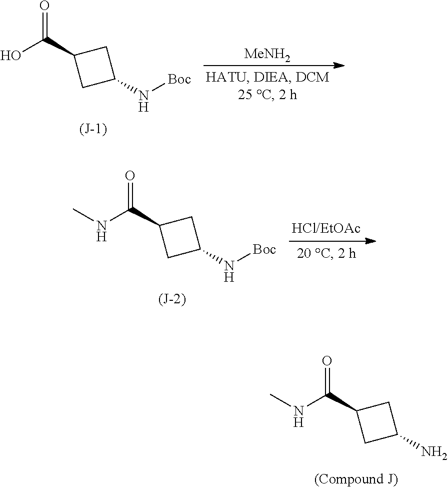

Synthesis of trans-tert-butyl ((1r,3r)-3-(methylcarbamoyl)cyclobutyl)carbamate (J-2). To a solution of trans-(1r,3r)-3-((tert-butoxycarbonyl)amino)cyclobutane-1-carboxylic acid (J-1) (5 g, 23.23 mmol, 1 eq) in DCM (70 mL) was added DIEA (9.01 g, 69.69 mmol, 12.14 mL, 3 eq), HATU (9.72 g, 25.55 mmol, 9.09 mL, 1.1 eq) and methanamine (1.73 g, 25.55 mmol, 1.1 eq, HCl). The mixture was stirred at 20 °C for 1 hr. LCMS showed trans-(1r,3r)-3-((tert-butoxycarbonyl)amino)cyclobutane-1-carboxylic acid (J-1) was consumed completely and 41% product with desired mass was detected. The reaction mixture was diluted with water (100 mL) and the aqueous phase was extracted with DCM (30 mL*3). The combined organic layers were dried over Na₂SO₄, filtered and the filtrate was concentrated to give a residue. The crude product was purified by re-crystallization from MTBE (50 mL) to give title Compound (J-2) (4.7 g, 20.59 mmol, 88.63% yield, 100% purity) as a white solid. LCMS (ES+): m/z 173.2 [M+H−56]⁺.

Synthesis of trans-(1r,3r)-3-amino-N-methylcyclobutane-1-carboxamide (Compound J). A mixture of trans-tert-butyl ((1r,3r)-3-(methylcarbamoyl)cyclobutyl)carbamate (J-2) (100 mg, 438.04 μmol, 1 eq) in HCl/EtOAc (4 M, 2.14 mL, 19.57 eq) was stirred at 20 °C for 2 hr. LCMS showed trans-tert-butyl ((1r,3r)-3-(methylcarbamoyl)cyclobutyl)carbamate (J-2) was consumed completely and the desired mass was detected. Vacuum distillation removes solvents to give title compound (Compound J) (72 mg, crude, HCl) as a white solid. LCMS (ES+): m/z 129.2 [M+H]⁺.

### Synthesis of 2-chloro-5-fluoro-4-(3-(2-methoxypyridin-3-yl) phenyl) pyrimidine (Compound K)

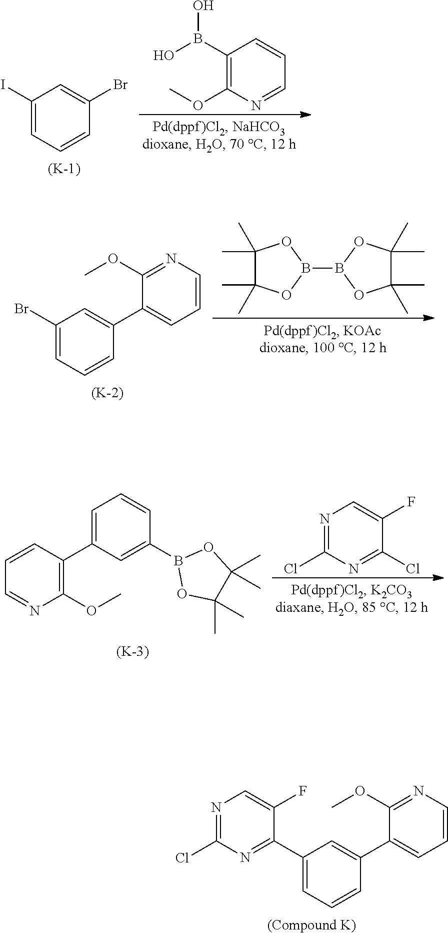

Synthesis of (2-methoxypyridin-3-yl) boronic acid (K-2). A mixture of 1-bromo-3-iodobenzene (2.11 kg, 7.47 mol, 952 mL, 1 eq), (2-methoxypyridin-3-yl)boronic acid (1.2 kg, 7.85 mol, 1.05 eq), Pd(dppf)Cl₂ (109 g, 149 mmol, 0.02 eq) and NaHCO₃ (1.26 kg, 14.9 mol, 2 eq) in dioxane (21 L) and H₂O (4.2 L) was degassed and purged with N₂ for 3 times at 15 °C, and then the mixture was stirred at 70 °C for 12 hours under N₂ atmosphere. LCMS showed (2-methoxy-3-pyridyl) boronic acid (K-1) was consumed completely and one main peak with desired mass was detected. The reaction mixture was partitioned between H₂O (4 L) and MTBE (12 L). The organic phase was separated, washed with 0.5 M NaOH (4 L*2) and brine (4 L*2), dried over anhydrous Na₂SO₄. The dried filtrate was concentrated under reduced pressure to give a residue. The residue was purified by MPLC (SiO₂, petroleum ether/ethyl acetate=5/1) to give the desired product (K-2) (1.88 kg, 7.12 mol, 95.3% yield) as a yellow oil. LCMS (ES⁺): m/z 264.1 [M+H]⁺.

Synthesis of 2-methoxy-3-(3-(4,4,5,5-tetramethyl-1,3,2-dioxaborolan-2-yl) phenyl) pyridine (K-3). A mixture of 3-(3-bromophenyl)-2-methoxy-pyridine (1.5 kg, 5.68 mol, 1 eq), 4, 4, 5, 5-tetramethyl-2-(4, 4, 5, 5-tetramethyl-1, 3, 2-dioxaborolan-2-yl)-1, 3, 2-dioxaborolane (1.87 kg, 7.38 mol, 1.3 eq), KOAc (1.45 kg, 14.7 mol, 2.6 eq) and Pd(dppf)Cl₂ (104 g, 142 mmol, 0.025 eq) in dioxane (15 L) was degassed and purged with N₂ for 3 times, and then the mixture was stirred at 100 °C for 12 hours under N₂ atmosphere. LCMS showed 3-(3-bromophenyl)-2-methoxy-pyridine was consumed completely and one main peak with desired mass was detected. The reaction mixture was filtered, and then the filtrate was used next step without further workup. LCMS (ES⁺): m/z 312.2 [M+H]⁺.

Synthesis of 2-chloro-5-fluoro-4-(3-(2-methoxypyridin-3-yl)phenyl)pyrimidine (Compound K). 2,4-dichloro-5-fluoropyrimidine (1.16 kg, 6.94 mol, 1.2 eq), K₂CO₃ (2.40 kg, 17.4 mol, 3 eq) and Pd(dppf)Cl₂ (106 g, 145 mmol, 0.025 eq) were added into the above reaction mixture. It was degassed and purged with N₂ for 3 times, and then the mixture was stirred at 85 °C for 12 hours under N₂ atmosphere. LCMS showed 2-methoxy-3-[3-(4, 4, 5, 5-tetramethyl-1, 3, 2-dioxaborolan-2-yl) phenyl]pyridine (K-3) was consumed completely and one main peak with desired mass was detected. The reaction mixture was partitioned between H₂O (4000 mL) and EtOAc (20 L). The organic phase was separated, washed with and brine (4 L * 2), dried over anhydrous Na₂SO₄. The dried filtrate was concentrated under reduced pressure to give a residue. The residue was purified by flash silica gel chromatography (ISCO®; 1500 g SepaFlash® Silica Flash Column, Eluent of 0~10% ethyl acetate/petroleum ether gradient @ 120 mL/min) to give desired product (Compound K) (1.1 kg, 3.38 mol, 58.4% yield, 97% purity) as a light-yellow solid. LCMS (ES⁺): m/z 316.1 [M+H]⁺.

### Synthesis of 1-(3-(2-chloro-5-fluoropyrimidin-4-yl)phenyl)pyridin-2(1H)-one (Compound L)

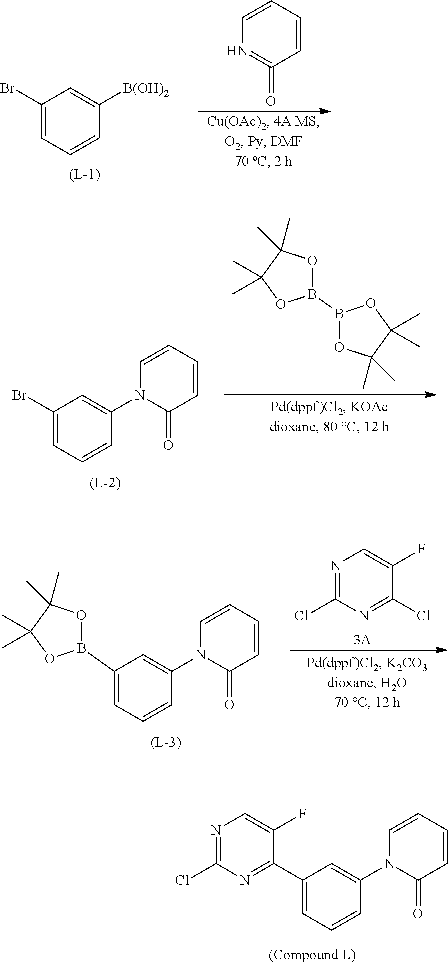

Synthesis of 1-(3-bromophenyl)pyridin-2(1H)-one (L-2). Sixteen reactions were conducted in parallel. (3-bromophenyl) boronic acid (250 g, 1.24 mol, 2 eq) was added into a solution of 1H-pyridin-2-one (59.2 g, 622 mmol, 1 eq), Cu(OAc)₂ (11.3 g, 62.2 mmol, 0.1 eq), Py (98.5 g, 1.24 mol, 100 mL, 2 eq) and 4 A molecular sieve (96 g) in DMF (2500 mL). O₂ (5% purity in N₂) was bubbled into the reaction mixture and stirred at 70 °C for 2 hours. HPLC showed (3-bromophenyl) boronic acid (L-1) remained and the desired compound was detected. Sixteen batches were combined for work-up. The reaction mixture was filtered and concentrated under reduced pressure to give a residue. Then the residue was diluted with H₂O (25 L) and extracted with EtOAc (25 L). The organic layer was washed with brine (20 L), dried over anhydrous Na₂SO₄, filtered, and concentrated under reduced pressure to give a residue. The residue was purified by flash silica gel chromatography (ISCO®; 13 kg SepaFlash®Silica Flash Column, Eluent of 0~100% EtOAc/petroleum ether gradient@ 200 mL/min) to give the title compound L-2 (1.3 kg, 5.20 mol, 52.2% yield) as a purple solid. LCMS (ES⁺): m/z 250.0 [M+H]⁺.

Synthesis of 1-(3-(4,4,5,5-tetramethyl-1,3,2-dioxaborolan-2-yl)phenyl)pyridin-2(1H)-one (L-3). A mixture of 1-(3-bromophenyl) pyridin-2-one (L-2) (1.3 kg, 5.20 mol, 1 eq), 4,4,5,5-tetramethyl-2-(4,4,5,5-tetramethyl-1,3,2-dioxaborolan-2-yl)-1,3,2-dioxaborolane (1.72 kg, 6.76 mol, 1.3 eq), KOAc (1.28 kg, 13.0 mol, 2.5 eq) and Pd(dppf)Cl₂ (95.1 g, 130 mmol, 0.025 eq) in dioxane (13 L) was degassed and purged with N₂ for 3 times, and then the mixture was stirred at 80 °C for 12 hours under N₂ atmosphere. LC-MS showed 1-(3-bromophenyl) pyridin-2-one (L-2) was consumed completely and desired mass was detected. The reaction mixture was filtered to give the title compound 3 (1540 g, crude) as a brown solid. LCMS (ES⁺): m/z 298.2 [M+H]⁺.

Synthesis of 1-(3-(2-chloro-5-fluoropyrimidin-4-yl)phenyl)pyridin-2(1H)-one (Compound L). A mixture of 2, 4-dichloro-5-fluoro-pyrimidine (952 g, 5.7 mol, 1.1 eq), 1-[3-(4,4,5,5-tetramethyl-1,3,2-dioxaborolan-2-yl)phenyl]pyridine-2-one (L-3) (1540 g, 5.18 mol, 1 eq), K₂CO₃ (2.15 kg, 15.6 mol, 3 eq) and Pd(dppf)Cl₂ (94.8 g, 130 mmol, 0.025 eq) in dioxane (25 L) and H₂O (5 L) was degassed and purged with N₂ for 3 times, and then the mixture was stirred at 70 °C for 12 hours under N₂ atmosphere. LC-MS showed 2, 4-dichloro-5-fluoro-pyrimidine (L-3) was consumed completely and desired mass was detected. The reaction mixture was filtered and concentrated under reduced pressure to give a residue. Then the residue was diluted with H₂O (25 L) and extracted with EtOAc (25 L). The organic layer was washed with brine (20 L), dried over Na₂SO₄, filtered, and concentrated under reduced pressure to give a residue. The crude product was triturated with MTBE (5 L) at 20 °C for 30 minutes. The filter cake was dried in vacuum to give the title compound (Compound L) (1100 g, 3.65 mol, 70.35% yield) as a pale-yellow solid. LCMS (ES⁺): m/z 302.1 [M+H]⁺.

### Synthesis of tert-butyl (1R,3S)-3-(((benzyloxy)carbonyl)amino)cyclohexane-1-carboxylate (Compound M) and tert-butyl (1S,3R)-3-(((benzyloxy)carbonyl)amino)cyclohexane-1-carboxylate (Compound N)

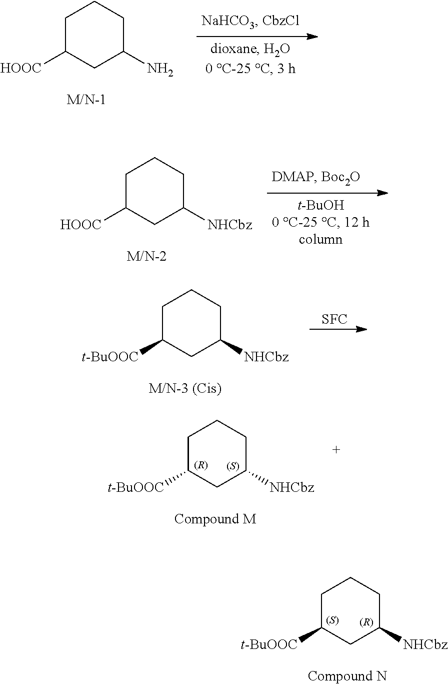

Synthesis of 3-(((benzyloxy)carbonyl)amino)cyclohexane-1-carboxylic acid (M/N-2). A mixture of 3-aminocyclohexane-1-carboxylic acid (M/N-1) (250 g, 1.75 mol, 1.00 eq), benzyl carbonochloridate (298 g, 1.75 mol, 249 mL, 1.00 eq) and Na₂CO₃ (555 g, 5.24 mol, 3.00 eq) in H₂O (2.50 L) and 1,4-dioxane (250 mL) was degassed and purged with N₂ for 3 times, and then the mixture was stirred at 25 °C for 12 hours under N₂ atmosphere. LCMS indicated 3-aminocyclohexane-1-carboxylic acid (M/N-1) was consumed completely and the desired product was detected. The reaction mixture was quenched by addition water (2000 mL) at 0 °C, and then extracted with MTBE (1000 mL*3). The combined organic layers were washed with brine (2000 mL), dried over anhydrous Na₂SO₄, filtered and concentrated under reduced pressure to get residue. The residue was purified by MPLC (SiO₂, petroleum ether/EtOAc=1/0 to 0/1) to give the desired product M/N-2 (377 g, 1.36 mol, 77.9% yield) as a white solid. LCMS (ES⁺): m/z 278.0 [M+H]⁺.

Synthesis of cis-tert-butyl)-3-(((benzyloxy)carbonyl)amino)cyclohexane-1-carboxylate (M/N-3 Cis). To a solution of 3-(((benzyloxy)carbonyl)amino)cyclohexane-1-carboxylic acid (300 g, 1.08 mol, 1.00 eq) in t-BuOH (3000 mL) was added Boc₂O (283 g, 1.30 mol, 298 mL, 1.20 eq) and DMAP (13.2 g, 108 mmol, 0.10 eq). The mixture was stirred at 35 °C for 12 hours. TLC showed 3-(((benzyloxy)carbonyl)amino)cyclohexane-1-carboxylic acid was consumed completely and one new spot was formed. The reaction mixture was diluted with H₂O (1000 mL) and extracted with EtOAc (1000 mL*3). The combined organic layers were washed with brine (2000 mL), dried over Na₂SO₄, filtered and concentrated under reduced pressure to give a residue. The residue was purified by column chromatography (SiO₂, Petroleum ether/Ethyl acetate=5/1 to 1/0) to give desired (M/N-3 Cis) (270 g, 810 mmol, 74.9% yield) as a white solid.

Synthesis of tert-butyl (1R,3S)-3-(((benzyloxy)carbonyl)amino)cyclohexane-1-carboxylate (Compound M) & tert-butyl (1S,3R)-3-(((benzyloxy)carbonyl)amino)cyclohexane-1-carboxylate (Compound N. The cis-tert-butyl-3-(((benzyloxy)carbonyl)amino)cyclohexane-1-carboxylate (M/N-3 cis) was separated by SFC (condition: column: DAICEL CHIRALCEL OD (250 mm*50 mm, 10 um); mobile phase: [CO₂—IPA (0.1% NH₃H₂O)]; B %: 25%, isocratic elution mode) to afford the desired Compound M (80 g, 229.86 mmol, 42.6% yield, 95.8% purity, 100% ee) as white solid, and Compound N (80 g, 235.86 mmol, 43.7% yield, 98.3% purity, 100% ee) as white solid. LCMS (ES⁺): m/z 334.2 [M+H]⁺.

### Synthesis of 2,5-dichloro-4-(4′-fluoro-[1,1′-biphenyl]-3-yl)pyrimidine (Compound O)

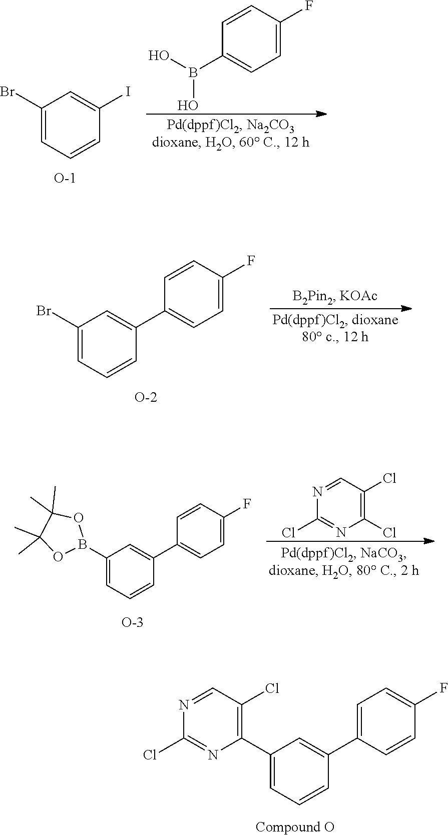

Synthesis of 3-bromo-4′-fluoro-1,1′-biphenyl (O-2). To a solution of 1-bromo-3-iodobenzene (O-1) (14.44 g, 51.05 mmol, 6.51 mL, 1 eq) and (4-fluorophenyl)boronic acid (5 g, 35.73 mmol, 0.7 eq) in dioxane (100 mL) and H₂O (10 mL) was added Na₂CO₃ (12.71 g, 153.15 mmol, 3 eq) followed by Pd(dppf)Cl₂ (1.87 g, 2.55 mmol, 0.05 eq). The reaction mixture was stirred at 60 °C for 12 h under N₂. TLC Plate 1 (Petroleum ether/Ethyl acetate=1/0) showed 1-bromo-3-iodobenzene (O-1) (R_{f}=0.8) was consumed completely and one new spot (R_{f}=0.7) formed. The residue was diluted with H₂O (100 mL) and extracted with EtOAc (100 mL*3). The combined organic layers were dried over Na₂SO₄, filtered and the filtrate was concentrated to give a residue. The residue was purified by flash silica gel chromatography (ISCO®; 20 g SepaFlash® Silica Flash Column, Eluent of 0~5% Ethyl acetate/Petroleum ethergradient @ 100 mL/min) to give title compound (O-2) (7 g, 27.88 mmol, 54.61% yield) as a yellow oil which was used in next step directly.

Synthesis of 2-(4′-fluoro-[1,1′-biphenyl]-3-yl)-4,4,5,5-tetramethyl-1,3,2-dioxaborolane (O-3). To a solution of 3-bromo-4′-fluoro-1,1′-biphenyl (O-2) (7 g, 27.88 mmol, 1 eq) and 4,4,5,5-tetramethyl-2-(4,4,5,5-tetramethyl-1,3,2-dioxaborolan-2-yl)-1,3,2-dioxaborolane (8.50 g, 33.45 mmol, 1.2 eq) in dioxane (80 mL) was added Pd(dppf)Cl₂ (2.04 g, 2.79 mmol, 0.1 eq) and KOAc (8.21 g, 83.63 mmol, 3 eq) at 25 °C. The reaction mixture was stirred at 80 °C for 12 h under N₂. TLC (Petroleum ether/Ethyl acetate=1/0) showed 3-bromo-4′-fluoro-1,1′-biphenyl (O-2) (R_{f}=0.60) was consumed completely and one new spot (R_{f}=0.55) formed. The reaction mixture was filtered. The filtrate was concentrated under reduced pressure to give title compound (O-3) (9.50 g, crude) as a yellow oil, which was used in next step directly.

Synthesis of 2,5-dichloro-4-(4′-fluoro-[1,1′-biphenyl]-3-yl)pyrimidine (Compound O). To a solution of 2,4,5-trichloropyrimidine (4.38 g, 23.90 mmol, 0.75 eq) and 2-(4′-fluoro-[1,1′-biphenyl]-3-yl)-4,4,5,5-tetramethyl-1,3,2-dioxaborolane (O-3) (9.5 g, 31.86 mmol, 1 eq) in dioxane (100 mL) and H₂O (10 mL) was slowly added Na₂CO₃ (10.13 g, 95.59 mmol, 3 eq) and Pd(dppf)Cl₂ (2.33 g, 3.19 mmol, 0.1 eq) at 25 °C. The reaction mixture was stirred at 80 °C for 2 h under N₂. LCMS showed 2-(4′-fluoro-[1,1′-biphenyl]-3-yl)-4,4,5,5-tetramethyl-1,3,2-dioxaborolane (O-3) was consumed completely and desired mass was detected. The residue was diluted with H₂O (200 mL) and extracted with EtOAc (200 mL*3). The combined organic layers were dried over Na₂SO₄, filtered and the filtrate was concentrated to give a residue. The residue was purified by flash silica gel chromatography (ISCO®; 40 g SepaFlash® Silica Flash Column, Eluent of 0~30% Ethyl acetate/Petroleum ethergradient @ 100 mL/min) to give title compound (Compound O) (3.2 g, 8.02 mmol, 25.17% yield, 80% purity) as a white solid. LCMS (ES+): m/z 318.9[M+H]⁺.

### Synthesis of 3-(3-(2,5-dichloropyrimidin-4-yl)phenyl)pyridin-2(1H)-one (Compound AA)

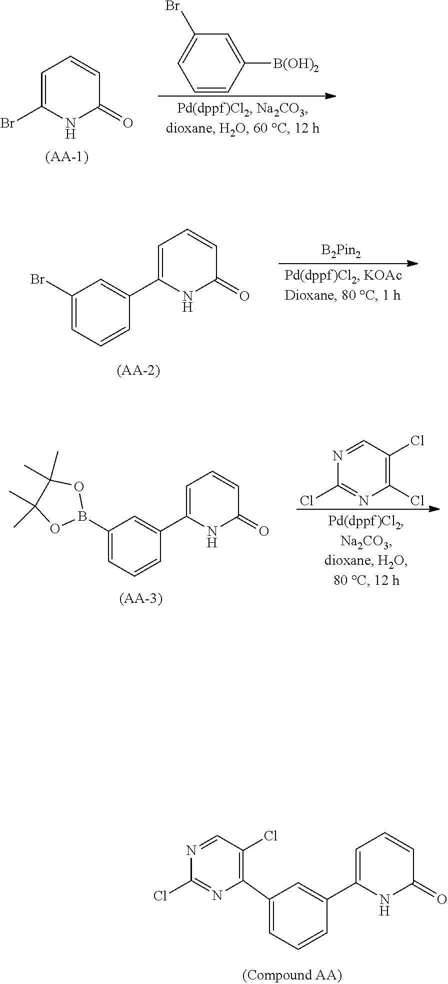

Synthesis of 6-(3-bromophenyl)pyridin-2(1H)-one (AA-2). To a solution of 6-bromopyridin-2(1H)-one (AA-1) (10 g, 57.47 mmol, 1 eq) in dioxane (100 mL) and H₂O (10 mL) was added Na₂CO₃ (18.27 g, 172.42 mmol, 3 eq), (3-bromophenyl) boronic acid (11.54 g, 57.47 mmol, 1 eq) and Pd(dppf)Cl₂ (2.10 g, 2.87 mmol, 0.05 eq) under N₂. The mixture was stirred at 60 °C for 12 h. LCMS showed 6-bromopyridin-2(1H)-one (AA-1) remaining and desired mass was detected. The reaction mixture was filtered and the filtrate was concentrated to give a residue. The residue was purified by flash silica gel chromatography (ISCO®; 40 g SepaFlash® Silica Flash Column, Eluent of 0~100% Ethyl acetate/Petroleum ethergradient @ 100 mL/min) to give title compound (AA-2) (9 g, 35.99 mmol, 62.62% yield) as a yellow solid. LCMS (ES+): m/z 250.1[M+H]⁺.

Synthesis of 6-(3-(4,4,5,5-tetramethyl-1,3,2-dioxaborolan-2-yl)phenyl)pyridin-2(1H)-one (AA-3). To a solution of 6-(3-bromophenyl)pyridin-2(1H)-one (AA-2) (9 g, 35.99 mmol, 1 eq) in dioxane (100 mL) was added 4,4,5,5-tetramethyl-2-(4,4,5,5-tetramethyl-1,3,2-dioxaborolan-2-yl)-1,3,2-dioxaborolane (9.14 g, 35.99 mmol, 1 eq), KOAc (10.60 g, 107.96 mmol, 3 eq) and Pd(dppf)Cl₂ (1.32 g, 1.80 mmol, 0.05 eq) under N₂. The mixture was stirred at 80 °C for 1 h. LCMS showed 6-(3-bromophenyl)pyridin-2(1H)-one (AA-2) was consumed completely and desired mass was detected. The reaction mixture was filtered and the filtrate was concentrated to give title compound (AA-3) (10 g, crude) as a black oil. LCMS (ES+): m/z 298.3 [M+H]⁺.

Synthesis of 6-(3-(2,5-dichloropyrimidin-4-yl)phenyl)pyridin-2(1H)-one (Compound AA). To a solution of the 2,4,5-trichloropyrimidine (4.63 g, 25.24 mmol, 0.75 eq) in dioxane (100 mL) and H₂O (10 mL) was added Pd(dppf)Cl₂ (1.23 g, 1.68 mmol, 0.05 eq), Na₂CO₃ (10.70 g, 100.96 mmol, 3 eq) and 6-(3-(4,4,5,5-tetramethyl-1,3,2-dioxaborolan-2-yl)phenyl)pyridin-2(1H)-one (AA-3) (10 g, 33.65 mmol, 1 eq) under N₂. The reaction mixture was stirred at 80 °C for 12 h. LCMS showed 6-(3-(4,4,5,5-tetramethyl-1,3,2-dioxaborolan-2-yl)phenyl)pyridin-2(1H)-one (AA-3) was consumed completely and desired mass was detected. The reaction mixture was filtered and the filtrate was concentrated to give a residue. The residue was purified by flash silica gel chromatography (ISCO®; 40 g SepaFlash® Silica Flash Column, Eluent of 0~100% Ethyl acetate/Petroleum ethergradient @ 100 m/min) to give title compound (Compound AA) (4 g, 12.57 mmol, 37.36% yield) as a white solid. LCMS (ES+): m/z 317.9 [M+H]⁺.

### Synthesis of 2,5-dichloro-4-(6-(4-fluorophenyl)pyridin-2-yl)pyrimidine (Compound BB)

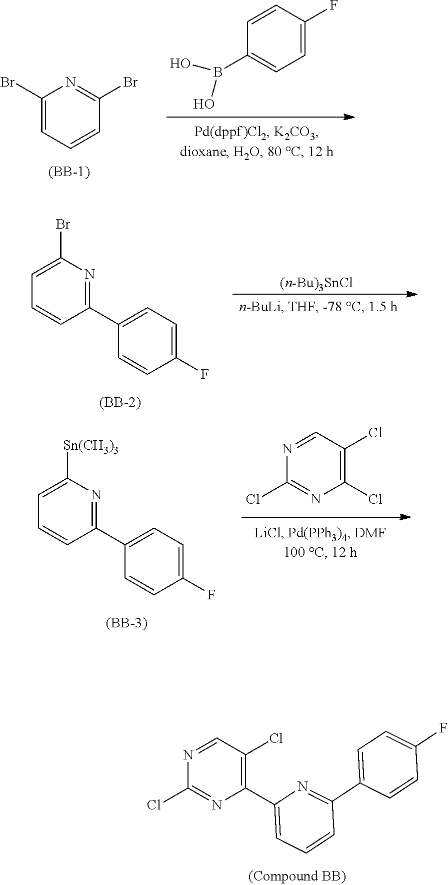

Synthesis of 2-bromo-6-(4-fluorophenyl)pyridine (BB-2). To a solution of 2,6-dibromopyridine (BB-1) (20 g, 84.43 mmol, 1.00 eq) and (4-fluorophenyl)boronic acid (11.22 g, 80.21 mmol, 0.95 eq) in dioxane (200 mL) and H₂O (20 mL) was added K₂CO₃ (23.34 g, 168.85 mmol, 2.00 eq) and Pd(dppf)Cl₂ (6.18 g, 8.44 mmol, 0.10 eq) and it was stirred at 80 °C for 12 h under N₂. LCMS showed 2,6-dibromopyridine (BB-1) was consumed and the desired mass was detected. The reaction was filtered and the filtrate was concentrated under reduced pressure to give a residue. The residue was purified by reversed-phase HPLC (neutral condition) to give title compound (BB-2) (8 g, 31.74 mmol, 37.59% yield) as a white solid. LCMS (ES+): m/z 252.1 [M+H]⁺.

Synthesis of 2-(4-fluorophenyl)-6-(trimethylstannyl)pyridine (BB-3). To a solution of 2-bromo-6-(4-fluorophenyl)pyridine (BB-2) (4.5 g, 17.85 mmol, 1 eq) in THF (40 mL) was added n-BuLi (2.5 M, 10.71 mL, 1.5 eq) at −78 °C and it was stirred at −78 °C for 0.5 h. tributyl(chloro)stannane (29.05 g, 89.26 mmol, 24.01 mL, 5 eq) was added and it was stirred at −78 °C for 1 h under N₂. LCMS showed 2-bromo-6-(4-fluorophenyl)pyridine (BB-2) was consumed and the desired mass was detected. The reaction was quenched by sat.aq.KF (20 mL) and PE (50 mL) added, filtered and the filtrate was concentrated under reduced pressure to give title compound (BB-3) (8.2 g, 17.74 mmol, 99.38% yield) as a brown oil. LCMS (ES+): m/z 337.9 [M+H]⁺.

Synthesis of 2, 5-dichloro-4-[6-(4-fluorophenyl)-2-pyridyl]pyrimidine (Compound BB). To a solution of 2-(4-fluorophenyl)-6-(trimethylstannyl)pyridine (BB-3) (7.2 g, 15.58 mmol, 1 eq) in DMF (60 mL) was added Pd(PPh₃)₄ (1.80 g, 1.56 mmol, 0.1 eq), 2,4,5-trichloropyrimidine (2.86 g, 15.58 mmol, 1 eq) and LiCl (1.85 g, 43.61 mmol, 894.10 L, 2.8 eq). The mixture was stirred at 100 °C for 12 h under N₂. LCMS showed 2-(4-fluorophenyl)-6-(trimethylstannyl)pyridine (BB-3) was consumed completely and desired mass was detected. The residue was diluted with H₂O (30 mL) and extracted with EtOAc (30 mL*3). The combined organic layers were dried over Na₂SO₄, filtered and the filtrate was concentrated to give a residue. The residue was purified by flash silica gel chromatography (ISCO®; 40 g SepaFlash® Silica Flash Column, Eluent of 0~30% Ethyl acetate/Petroleum ethergradient @ 80 mL/min) to give title compound (Compound BB) (1.7 g, 5.31 mmol, 34.09% yield) as a yellow solid. LCMS (ES+): m/z 319.9 [M+H]⁺.

### Synthesis of 2,5-dichloro-4-(2-(4-fluorophenyl)pyridin-4-yl)pyrimidine (Compound CC)

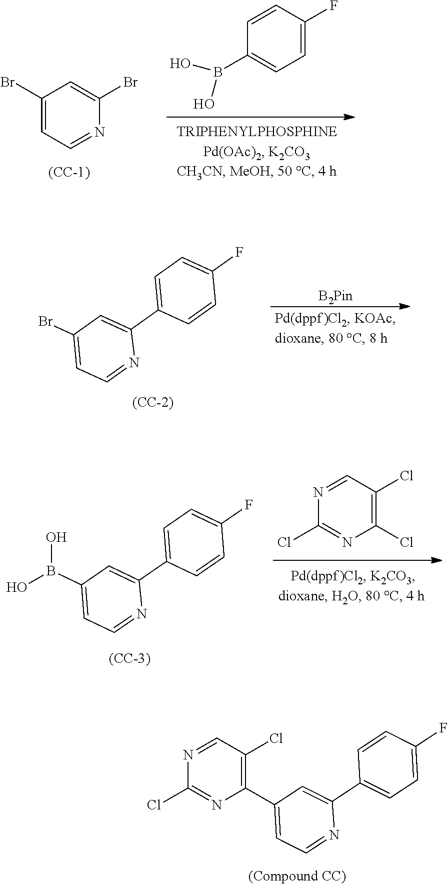

Synthesis of 4-bromo-2-(4-fluorophenyl) pyridine (CC-2). To a solution of 2,4-dibromopyridine (CC-1) (5 g, 21.11 mmol, 1 eq) in CH₃CN (40 mL) and MeOH (20 mL) was added K₂CO₃ (5.83 g, 42.21 mmol, 2 eq), Pd(OAc)₂ (473.86 mg, 2.11 mmol, 0.1 eq), TRIPHENYLPHOSPHINE (1.11 g, 4.22 mmol, 0.2 eq) and (4-fluorophenyl)boronic acid (4.69 g, 21.11 mmol, 1 eq). The mixture was stirred at 50 °C for 4 h under N₂. LCMS showed 2,4-dibromopyridine (CC-1) was consumed completely and desired mass was detected. The residue was diluted with H₂O (30 mL) and extracted with EtOAc (30 mL*3). The combined organic layers were dried over Na₂SO₄, filtered and the filtrate was concentrated to give a residue. The residue was purified by flash silica gel chromatography (ISCO®; 40 g SepaFlash® Silica Flash Column, Eluent of 0~10% Ethyl acetate/Petroleum ethergradient @ 80 mL/min) to give title compound (CC-2) (3.5 g, 13.88 mmol, 65.78% yield) as a yellow oil. LCMS (ES⁺): m/z 252.1 [M+H]⁺.

Synthesis of (2-(4-fluorophenyl)pyridin-4-yl)boronic acid (CC-3). To a solution of 4-bromo-2-(4-fluorophenyl)pyridine (CC-2) (3 g, 11.90 mmol, 1 eq) in dioxane (40 mL) was added KOAc (3.50 g, 35.70 mmol, 3 eq) and Pd(dppf)Cl₂ (870.79 mg, 1.19 mmol, 0.1 eq) and 4,4,5,5-tetramethyl-2-(4,4,5,5-tetramethyl-1,3,2-dioxaborolan-2-yl)-1,3,2-dioxaborolane (3.02 g, 11.90 mmol, 1 eq). The mixture was stirred at 80 °C for 8 h. LCMS showed 4-bromo-2-(4-fluorophenyl)pyridine (CC-2) was consumed completely and desired mass was detected. The reaction mixture was concentrated to give title compound (CC-3) (3.5 g, 11.70 mmol, 98.31% yield) as a yellow oil, which was used in next step directly. LCMS (ES+): m/z 218.1 [M+H]⁺.

Synthesis of 2,5-dichloro-4-(2-(4-fluorophenyl)pyridin-4-yl)pyrimidine (Compound CC). To a solution of (2-(4-fluorophenyl)pyridin-4-yl)boronic acid (CC-3) (3.5 g, 11.70 mmol, 1 eq) in dioxane (50 mL) and Water (5 mL) was added K₂CO₃ (3.23 g, 23.40 mmol, 2 eq), 2,4,5-trichloropyrimidine (2.15 g, 11.70 mmol, 1 eq) and Pd(dppf)Cl₂ (856.09 mg, 1.17 mmol, 0.1 eq). The mixture was stirred at 80 °C for 4 h under N₂. LCMS showed (2-(4-fluorophenyl)pyridin-4-yl)boronic acid (CC-3) was consumed completely and desired mass was detected. The reaction mixture was diluted with H₂O (30 mL) and extracted with EtOAc (20 mL*3). The combined organic layers were dried over Na₂SO₄, filtered and the filtrate was concentrated to give a residue. The residue was purified by flash silica gel chromatography (ISCO®; 40 g SepaFlash® Silica Flash Column, Eluent of 0~30% Ethyl acetate/Petroleum ethergradient @ 80 mL/min). TLC Plate 1 (Petroleum ether/Ethyl acetate=2/1, R_{f}=0.60) to give title compound (Compound CC) (2.2 g, 6.87 mmol, 58.73% yield) as a white solid. LCMS (ES⁺): m/z 320.1 [M+H]⁺.

### Synthesis of 5-(2,5-dichloropyrimidin-4-yl)-1-(4-fluorophenyl)pyridin-2(1H)-one (Compound DD)

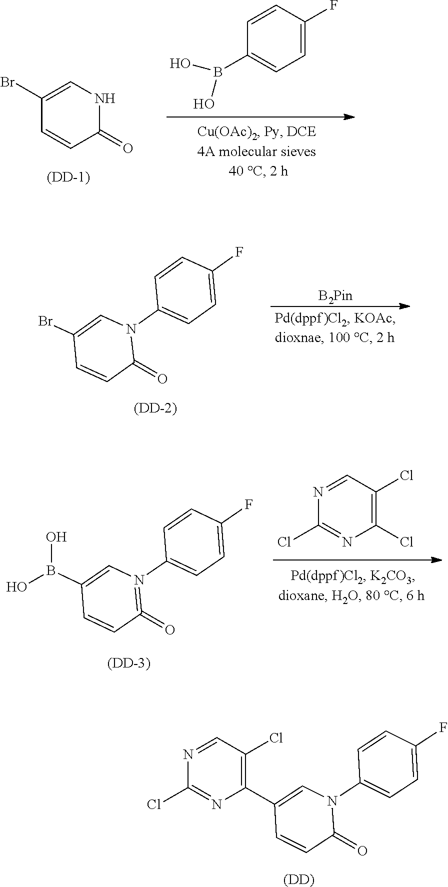

Synthesis of 5-bromo-1-(4-fluorophenyl)pyridin-2-one (DD-2). To a solution of 5-bromo-1H-pyridin-2-one (5 g, 28.74 mmol, 1 eq) (DD-1) in DCE (70 mL) was added Cu(OAc)₂ (5.22 g, 28.74 mmol, 1 eq), 4 A MOLECULAR SIEVE (2 g, 100.00 mmol, 3.48 eq), Py (4.55 g, 57.47 mmol, 4.64 mL, 2 eq) and (4-fluorophenyl)boronic acid (4.02 g, 28.74 mmol, 1 eq). The mixture was stirred at 40 °C for 2 h. LCMS showed 5-bromo-1H-pyridin-2-one (DD-1) was consumed completely and desired mass was detected. The reaction mixture was concentrated to give a residue. The residue was diluted with H₂O (30 ml) and extracted with EtOAc (30 mL*3). The combined organic layers were dried over Na₂SO₄, filtered and the filtrate was concentrated to give a residue. The residue was purified by flash silica gel chromatography (ISCO®; 40 g SepaFlash® Silica Flash Column, Eluent of 0~30% Ethyl acetate/Petroleum ethergradient @ 80 mL/min) to give title compound (DD-2) (7 g, 26.11 mmol, 90.87% yield) as a white solid. LCMS (ES⁺): m/z 267.9 [M+H]⁺.

Synthesis of (1-(4-fluorophenyl)-6-oxo-1,6-dihydropyridin-3-yl)boronic acid (DD-3). To a solution of 5-bromo-1-(4-fluorophenyl)pyridin-2-one (DD-2) (4 g, 14.92 mmol, 1 eq) in dioxane (60 mL) was added Pd(dppf)Cl₂ (1.09 g, 1.49 mmol, 0.1 eq), KOAc (4.39 g, 44.76 mmol, 3 eq) and 4,4,5,5-tetramethyl-2-(4,4,5,5-tetramethyl-1,3,2-dioxaborolan-2-yl)-1,3,2-dioxaborolane (3.79 g, 14.92 mmol, 1 eq). The mixture was stirred at 100 °C for 2 h under N₂ atmosphere. LCMS showed 5-bromo-1-(4-fluorophenyl)pyridin-2-one (DD-2) was consumed completely and 32% peak with desired mass was detected. The reaction mixture was concentrated to give title compound (DD-3) (4.7 g, 14.91 mmol, 99.95% yield) as a yellow oil, which was used in next step directly. LCMS (ES⁺): m/z 234.0 [M+H]⁺.

Synthesis of 5-(2,5-dichloropyrimidin-4-yl)-1-(4-fluorophenyl)pyridin-2-one (Compound DD). To a solution of [1-(4-fluorophenyl)-6-oxo-3-pyridyl]boronic acid (DD-3) (3.4 g, 14.59 mmol, 1 eq) in dioxane (100 mL) and H₂O (10 mL) was added K₂CO₃ (6.05 g, 43.78 mmol, 3 eq), 2,4,5-trichloropyrimidine (2.68 g, 14.59 mmol, 1 eq) and Pd(dppf)Cl₂ (1.07 g, 1.46 mmol, 0.1 eq). The mixture was stirred at 80 °C for 6 h under N₂ atmosphere. LCMS showed [1-(4-fluorophenyl)-6-oxo-3-pyridyl]boronic acid (DD-3) was consumed completely and desired mass was detected. The reaction mixture was diluted with H₂O (30 mL) and extracted with EtOAc (30 mL*3). The combined organic layers were dried over Na₂SO₄, filtered and the filtrate was concentrated to give a residue. The residue was purified by flash silica gel chromatography (ISCO®; 40 g SepaFlash®Silica Flash Column, Eluent of 0-40% Ethyl acetate/Petroleum ethergradient @ 40 mL/min) to give title compound (Compound DD) (1.5 g, 4.46 mmol, 30.58% yield) as a white solid. LCMS (ES⁺): m/z 336.0 [M+H]⁺.

### Synthesis of 1-cyclopropyl-5-(2,5-dichloropyrimidin-4-yl)pyridin-2-one (Compound EE)

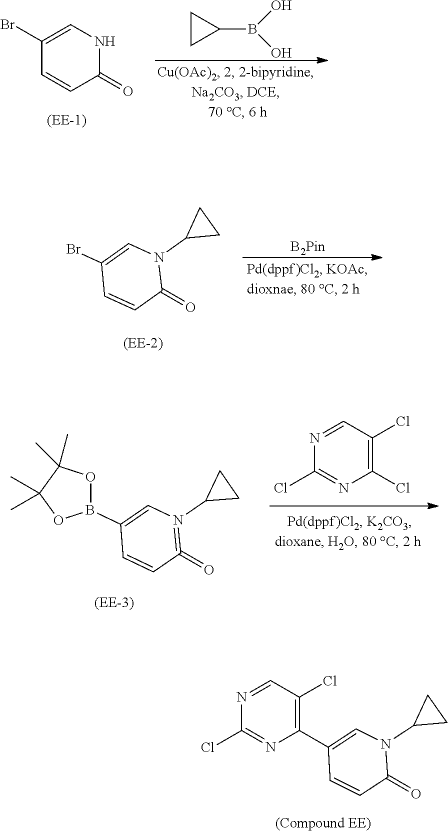

Synthesis of 5-bromo-1-cyclopropyl-pyridin-2-one (EE-2). To a solution of 5-bromo-1H-pyridin-2-one (EE-1) (5 g, 28.74 mmol, 1 eq) in DCE (120 mL) was added cyclopropylboronic acid (7.41 g, 86.21 mmol, 3 eq), 2-(2-pyridyl)pyridine (4.94 g, 31.61 mmol, 1.1 eq), Na₂CO₃ (9.14 g, 86.21 mmol, 3 eq) and Cu(OAc)₂ (5.22 g, 28.74 mmol, 1 eq). The reaction stirred at 70 °C for 6 h. LCMS showed 3.5% of 5-bromo-1H-pyridin-2-one (EE-1) was remaining and 28% product with desired mass was detected. After filtration, vacuum distillation removes solvents to obtain crude products. The residue was purified by flash silica gel chromatography (ISCO®; 40 g SepaFlash® Silica Flash Column, Eluent of 0~100% Ethyl alcohol/Petroleum ethergradient @ 80 mL/min) to give title compound (EE-2) (4.4 g, 18.71 mmol, 65.09% yield, 91% purity) as a white solid. LCMS (ES+): m/z 213.9 [M+H]⁺.

Synthesis of 1-cyclopropyl-5-(4,4,5,5-tetramethyl-1,3,2-dioxaborolan-2-yl)pyridin-2-one (EE-3). To a solution of 5-bromo-1-cyclopropyl-pyridin-2-one (EE-2) (2.8 g, 13.08 mmol, 1 eq) in dioxane (30 mL) was added 4,4,5,5-tetramethyl-2-(4,4,5,5-tetramethyl-1,3,2-dioxaborolan-2-yl)-1,3,2-dioxaborolane (3.65 g, 14.39 mmol, 1.1 eq), KOAc (3.85 g, 39.24 mmol, 3 eq) and Pd(dppf)Cl₂ (957.11 mg, 1.31 mmol, 0.1 eq) under N₂. The reaction stirred at 80 °C for 2 h. LCMS showed 5-bromo-1-cyclopropyl-pyridin-2-one (EE-2) was consumed completely and the desired mass was detected. After filtration, vacuum distillation removes solvents to give title compound (EE-3) (3.4 g, crude) as a black solid. LCMS (ES⁺): m/z 262.1 [M+H]⁺.

Synthesis of 1-cyclopropyl-5-(2,5-dichloropyrimidin-4-yl)pyridin-2-one (Compound EE). To a stirred solution of 1-cyclopropyl-5-(4,4,5,5-tetramethyl-1,3,2-dioxaborolan-2-yl)pyridin-2-one (EE-3) (3 g, 11.49 mmol, 1 eq) in dioxane (40 mL) and H₂O (4 mL) was added K₂CO₃ (4.76 g, 34.47 mmol, 3 eq), Pd(dppf)Cl₂ (840.64 mg, 1.15 mmol, 0.1 eq) and 2,4,5-trichloropyrimidine (2.11 g, 11.49 mmol, 1 eq) under N₂. The reaction stirred at 80 °C over 2 h. LCMS showed 1-cyclopropyl-5-(4,4,5,5-tetramethyl-1,3,2-dioxaborolan-2-yl)pyridin-2-one (EE-3) was consumed completely and the desired mass was detected. The reaction was concentrated to give a residue. The residue was purified by flash silica gel chromatography (ISCO; 40 g SepaFlash Silica Flash Column, Eluent of 0~100% Ethyl acetate/Petroleum ethergradient @ 80 mL/min) to give title compound (Compound EE) (2.76 g, 8.26 mmol, 71.87% yield, 84.4% purity) as a white solid. LCMS (ES⁺): m/z 281.9 [M+H]⁺.

### Synthesis of 2,5-dichloro-4-(1-cyclopropylpyrazol-4-yl)pyrimidine (Compound FF)

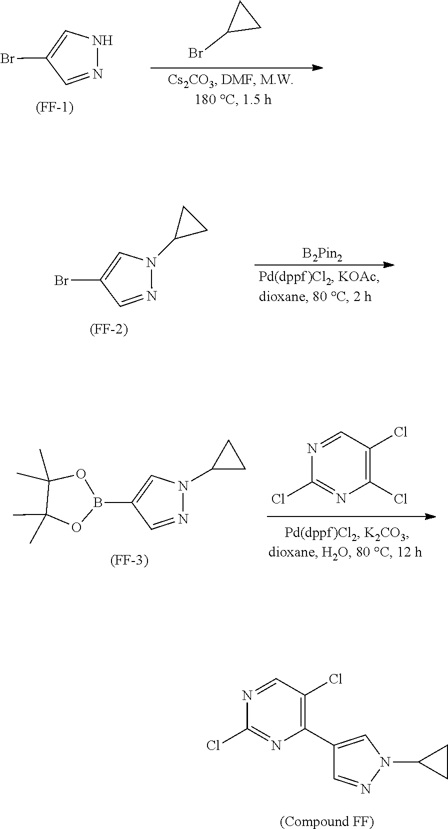

Synthesis of 4-bromo-1-cyclopropyl-pyrazole (FF-2). To a solution of 4-bromo-1H-pyrazole (FF-1) (4 g, 27.22 mmol, 1 eq) in DMF (5 mL) was added bromocyclopropane (4.94 g, 40.82 mmol, 3.27 mL, 1.5 eq), Cs₂CO₃ (13.30 g, 40.82 mmol, 1.5 eq), the mixture was stirred at 180 °C for 1.5 h under M.W. LCMS showed 4-bromo-1H-pyrazole (FF-1) was consumed completely and the desired mass was detected. The reaction mixture was concentrated to give a residue. The residue was diluted with H₂O (30 mL) and extracted with EtOAc (30 mL*3). The combined organic layers were dried over Na₂SO₄, filtered and the filtrate was concentrated to give a residue. The residue was purified by flash silica gel chromatography (ISCO®; 40 g SepaFlash® Silica Flash Column, Eluent of 0~25% Ethyl acetate/Petroleum ethergradient @ 60 mL/min) to give title compound (FF-2) (2 g, 10.69 mmol, 39.29% yield) as a colorless liquid. LCMS (ES+): m/z 186.9 [M+H]⁺.

Synthesis of 1-cyclopropyl-4-(4,4,5,5-tetramethyl-1,3,2-dioxaborolan-2-yl)pyrazole (FF-3). To a solution of 4-bromo-1-cyclopropyl-pyrazole (FF-2) (1.8 g, 9.62 mmol, 1 eq) in dioxane (20 mL) was added 4,4,5,5-tetramethyl-2-(4,4,5,5-tetramethyl-1,3,2-dioxaborolan-2-yl)-1,3,2-dioxaborolane (2.93 g, 11.55 mmol, 1.2 eq), KOAc (3.78 g, 38.50 mmol, 4 eq) and Pd(dppf)Cl₂ (704.18 mg, 962.38 μmol, 0.1 eq), the mixture was stirred at 80 °C for 2 h. LCMS showed 4-bromo-1-cyclopropyl-pyrazole (FF-2) was consumed completely and the desired mass was detected. The reaction mixture was filtered and concentrated to give title compound (FF-3) (2.25 g, 9.61 mmol, 100.00% yield) as a black solid. LCMS (ES+): m/z 235.1 [M+H]⁺.

Synthesis of 2,5-dichloro-4-(1-cyclopropylpyrazol-4-yl)pyrimidine (Compound FF) To a solution 1-cyclopropyl-4-(4,4,5,5-tetramethyl-1,3,2-dioxaborolan-2-yl)pyrazole (FF-3) (2.25 g, 9.61 mmol, 1 eq) in dioxane (25 mL) and H₂O (2.5 mL) was added 2,4,5-trichloropyrimidine (2.12 g, 11.53 mmol, 1.2 eq), Pd(dppf)Cl₂ (703.26 mg, 961.12 μmol, 0.1 eq) and K₂CO₃ (3.99 g, 28.83 mmol, 3 eq) at 25 °C, the mixture was stirred at 80 °C for 12 h under N₂. LCMS showed 1-cyclopropyl-4-(4,4,5,5-tetramethyl-1,3,2-dioxaborolan-2-yl)pyrazole (FF-3) was consumed completely and the desired mass was detected. The reaction mixture was concentrated to give a residue. The residue was diluted with H₂O (30 mL) and extracted with EtOAc (30 mL*3). The combined organic layers were dried over Na₂SO₄, filtered and the filtrate was concentrated to give a residue. The residue was purified by flash silica gel chromatography (ISCO®; 20 g SepaFlash® Silica Flash Column, Eluent of 0-30% Ethyl acetate/Petroleum ethergradient @ 60 mL/min) to give title compound (Compound FF) (1.6 g, 6.27 mmol, 65.26% yield) as a yellow solid. LCMS (ES+): m/z 255.1 [M+H]⁺.

### Synthesis of 2,5-dichloro-4-(1-cyclohexyl-1H-pyrazol-4-yl)pyrimidine (Compound GG)

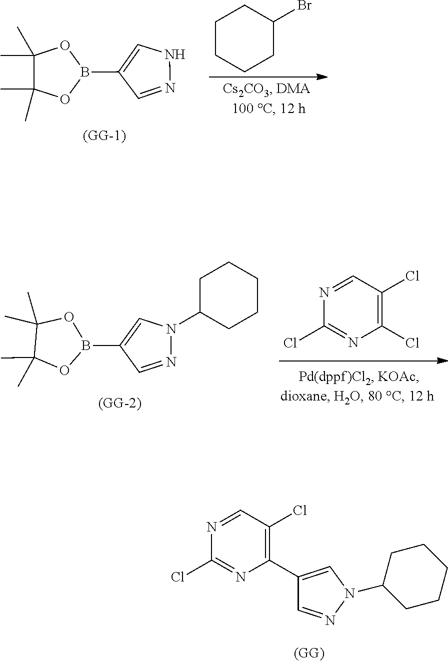

Synthesis of 1-cyclohexyl-4-(4,4,5,5-tetramethyl-1,3,2-dioxaborolan-2-yl)pyrazole (GG-2). To a solution of 4-(4,4,5,5-tetramethyl-1,3,2-dioxaborolan-2-yl)-1H-pyrazole (GG-1) (10 g, 51.54 mmol, 1 eq) in DMA (50 mL) was added bromocyclo hexane (42.02 g, 257.68 mmol, 31.73 mL, 5 eq) and Cs₂CO₃ (50.37 g, 154.61 mmol, 3 eq), the mixture was stirred at 100 °C for 12 h. LCMS showed 4-(4,4,5,5-tetramethyl-1,3,2-dioxaborolan-2-yl)-1H-pyrazole (GG-1) was consumed completely and the desired mass was detected. The reaction mixture was concentrated to give a residue. The residue was diluted with H₂O (100 mL) and extracted with EtOAc (100 mL*3). The combined organic layers were dried over Na₂SO₄, filtered and the filtrate was concentrated to give a residue. The residue was purified by flash silica gel chromatography (ISCO®; 40 g SepaFlash® Silica Flash Column, Eluent of 0~20% Ethyl acetate/Petroleum ethergradient @ 60 mL/min) to give title compound (GG-2) (2 g, 7.24 mmol, 14.05% yield) as a yellow oil. LCMS (ES+): m/z 277.1 [M+H]⁺.

Synthesis of 2,5-dichloro-4-(1-cyclohexyl-1H-pyrazol-4-yl)pyrimidine (Compound GG). To a solution 1-cyclohexyl-4-(4,4,5,5-tetramethyl-1,3,2-dioxaborolan-2-yl)pyrazole (GG-2) (3 g, 10.86 mmol, 1 eq) in dioxane (30 mL) and H₂O (3 mL) was added 2,4,5-trichloropyrimidine (1.99 g, 10.86 mmol, 1 eq), Pd(dppf)Cl₂ (794.81 mg, 1.09 mmol, 0.1 eq) and KOAc (3.20 g, 32.59 mmol, 3 eq) at 25 °C, the mixture was stirred at 80 °C for 12 h under N₂. LCMS showed 1-cyclohexyl-4-(4,4,5,5-tetramethyl-1,3,2-dioxaborolan-2-yl)pyrazole (GG-2) was consumed completely and the desired mass was detected. The reaction mixture was concentrated to give a residue. The residue was diluted with H₂O (20 mL) and extracted with EtOAc (20 mL*3). The combined organic layers were dried over Na₂SO₄, filtered and the filtrate was concentrated to give a residue. The residue was purified by flash silica gel chromatography (ISCO®; 40 g SepaFlash® Silica Flash Column, Eluent of 0-25% Ethyl acetate/Petroleum ethergradient @ 60 mL/min) to give title compound (Compound GG) (1.6 g, 5.38 mmol, 49.56% yield) as a yellow solid. LCMS (ES+): m/z 296.9 [M+H]⁺.

### Synthesis of 2,5-dichloro-4-(1-cyclohexyl-1H-pyrazol-3-yl)pyrimidine (Compound HH)

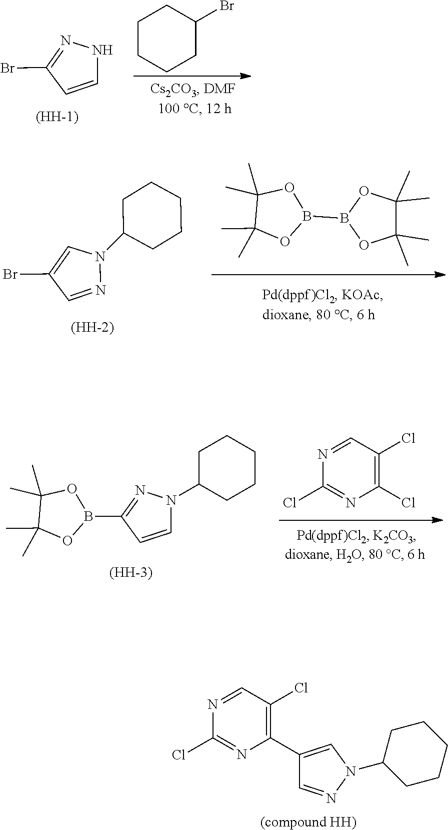

Synthesis of 3-bromo-1-cyclohexyl-pyrazole (HH-2). To a solution of 3-bromo-1H-pyrazole (HH-1) (10 g, 68.04 mmol, 1 eq) in DMF (100 mL) was added bromocyclohexane (44.38 g, 272.16 mmol, 33.52 mL, 4 eq) and Cs₂CO₃ (110.84 g, 340.20 mmol, 5 eq) at 25 °C, the mixture was stirred at 100 °C for 12 h. LCMS showed 3-bromo-1H-pyrazole (HH-1) was consumed completely and the desired mass was detected. The reaction mixture was concentrated to give a residue. The residue was diluted with H₂O (50 mL) and extracted with EtOAc (50 mL*3). The combined organic layers were dried over Na₂SO₄, filtered and the filtrate was concentrated to give a residue. The residue was purified by flash silica gel chromatography (ISCO®; 40 g SepaFlash® Silica Flash Column, Eluent of 0~15% Ethyl acetate/Petroleum ethergradient @ 60 mL/min) to give title compound (HH-2) (12 g, 64.58 mmol, 76.98% yield) as a colorless clear liquid. LCMS (ES⁺): m/z 229.0 [M+H]⁺.

Synthesis of 1-cyclohexyl-3-(4,4,5,5-tetramethyl-1,3,2-dioxaborolan-2-yl)pyrazole (HH-3). To a solution of 3-bromo-1-cyclohexyl-pyrazole (HH-2) (5 g, 21.82 mmol, 1 eq) in dioxane (50 mL) was added 4,4,5,5-tetramethyl-2-(4,4,5,5-tetramethyl-1,3,2-dioxaborolan-2-yl)-1,3,2-dioxaborolane (6.65 g, 26.19 mmol, 1.2 eq), KOAc (10.71 g, 109.11 mmol, 5 eq) and Pd(dppf)Cl₂ (3.19 g, 4.36 mmol, 0.2 eq) the mixture was stirred at 80 °C for 6 h. LCMS showed 3-bromo-1-cyclohexyl-pyrazole (HH-2) was consumed completely and desired mass was detected. The reaction mixture was filtered and concentrated to give title compound (HH-3) (6 g, 37.82 mmol, 99.69% yield) as a black oil, which was used in next step directly. LCMS (ES⁺): m/z 277.3 [M+H]⁺.

Synthesis of 2,5-dichloro-4-(1-cyclohexyl-1H-pyrazol-3-yl)pyrimidine (Compound HH). To a solution of 1-cyclohexyl-3-(4,4,5,5-tetramethyl-1,3,2-dioxaborolan-2-yl)pyrazole (HH-3) (6 g, 21.72 mmol, 1 eq) in dioxane (25 mL) and H₂O (2.5 mL) was added 2,4,5-trichloropyrimidine (4.78 g, 26.07 mmol, 1.2 eq), Pd(dppf)Cl₂ (1.59 g, 2.17 mmol, 0.1 eq) and K₂CO₃ (9.01 g, 65.17 mmol, 3 eq), the mixture was stirred at 80 °C for 6 h under N₂. LCMS showed 1-cyclohexyl-3-(4,4,5,5-tetramethyl-1,3,2-dioxaborolan-2-yl)pyrazole (HH-3) was consumed completely and the desired mass was detected. The reaction mixture was concentrated to give a residue. The residue was diluted with H₂O (30 mL) and extracted with EtOAc (30 mL*3). The combined organic layers were dried over Na₂SO₄, filtered and the filtrate was concentrated to give a residue. The residue was purified by flash silica gel chromatography (ISCO®; 40 g SepaFlash® Silica Flash Column, Eluent of 0~30% Ethyl acetate/Petroleum ethergradient @ 60 mL/min) to give title compound (Compound HH) (1.2 g, 4.04 mmol, 18.59% yield) as a white solid. LCMS (ES⁺): m/z 297.1 [M+H]⁺.

### Synthesis of 2,5-dichloro-4-(5-cyclopropyl-1H-pyrazol-3-yl)pyrimidine (Compound JJ)

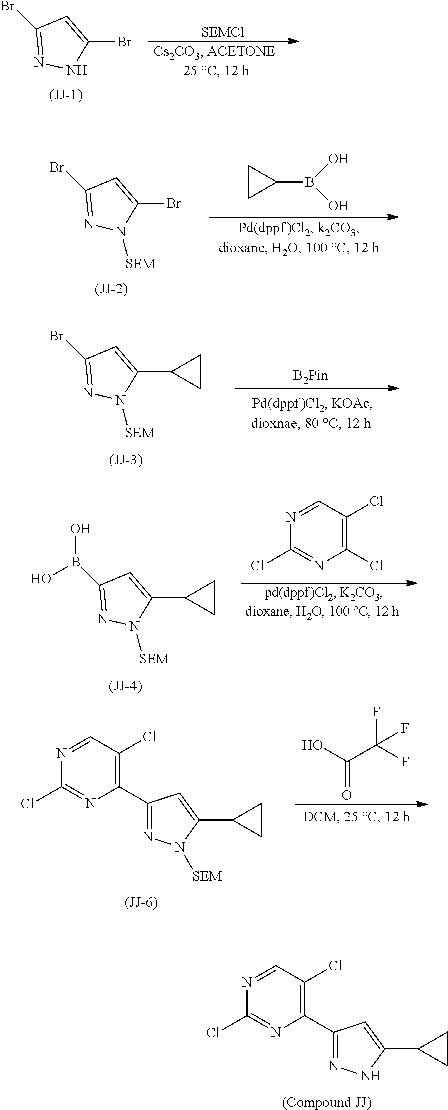

Synthesis of 2-[(3,5-dibromopyrazol-1-yl)methoxy]ethyl-trimethyl-silane (JJ-2). To a solution of 3,5-dibromo-1H-pyrazole (JJ-1) (15 g, 66.41 mmol, 1 eq) in ACETONE (30 mL) was added 2-(chloromethoxy) ethyl-trimethyl-silane (38.75 g, 232.44 mmol, 41.14 mL, 3.5 eq) and Cs₂CO₃ (108.19 g, 332.05 mmol, 5 eq) at 0 °C, the mixture was stirred at 25 °C for 12 h. LCMS showed 3,5-dibromo-1H-pyrazole (JJ-1) was consumed completely and the desired mass was detected. The reaction mixture was concentrated to give a residue. The residue was diluted with H₂O (50 mL) and extracted with EtOAc (50 mL*3). The combined organic layers were dried over Na₂SO₄, filtered and the filtrate was concentrated to give a residue. The residue was purified by flash silica gel chromatography (ISCO®; 40 g SepaFlash® Silica Flash Column, Eluent of 0~15% Ethyl acetate/Petroleum ethergradient @ 60 mL/min) to give title compound (JJ-2) (23 g, 64.58 mmol, 97.25% yield) as a colorless clear liquid. LCMS (ES⁺): m/z 354.9 [M+H]⁺.

Synthesis of 2-[(3-bromo-5-cyclopropyl-pyrazol-1-yl)methoxy]ethyl-trimethyl-silane (JJ-3). To a solution of 2-[(3,5-dibromopyrazol-1-yl)methoxy]ethyl-trimethyl-silane (JJ-2) (23 g, 64.58 mmol, 1 eq) in dioxane (50 mL) and H₂O (5 mL) was added cyclopropylboronic acid (7.21 g, 83.96 mmol, 1.3 eq), Pd(dppf)Cl₂ (4.73 g, 6.46 mmol, 0.1 eq) and K₂CO₃ (26.78 g, 193.75 mmol, 3 eq) at 25 °C, the mixture was stirred at 100 °C for 12 h under N₂. LC-MS showed 2-[(3,5-dibromopyrazol-1-yl)methoxy]ethyl-trimethyl-silane (JJ-2) was consumed completely and desired mass was detected. The residue was diluted with H₂O (100 mL) and extracted with EtOAc (50 mL*3). The combined organic layers were dried over Na₂SO₄, filtered and the filtrate was concentrated to give a residue. The residue was purified by flash silica gel chromatography (ISCO®; 120 g SepaFlash® Silica Flash Column, Eluent of 0~100% Ethyl acetate/Petroleum ethergradient @ 80 mL/min) to give title compound (JJ-3) (12 g, 37.82 mmol, 58.56% yield) as a colorless liquid. LCMS (ES⁺): m/z 317.0 [M+H]⁺.

Synthesis of (5-cyclopropyl-1-((2-(trimethylsilyl)ethoxy)methyl)-1H-pyrazol-3-yl)boronic acid (JJ-4). To a solution of 2-[(3-bromo-5-cyclopropyl-pyrazol-1-yl)methoxy]ethyl-trimethyl-silane (JJ-3) (12 g, 37.82 mmol, 1 eq) in dioxane (5 mL) was added 4,4,5,5-tetramethyl-2-(4,4,5,5-tetramethyl-1,3,2-dioxaborolan-2-yl)-1,3,2-dioxaborolane (11.52 g, 45.38 mmol, 1.2 eq), KOAc (14.85 g, 151.28 mmol, 4 eq) and Pd(dppf)Cl₂ (2.77 g, 3.78 mmol, 0.1 eq) at 25 °C, the mixture was stirred at 80 °C for 12 h under N₂. LCMS showed 2-[(3-bromo-5-cyclopropyl-pyrazol-1-yl)methoxy]ethyl-trimethyl-silane (JJ-3) was consumed completely and the desired mass was detected. The reaction mixture was filtered and concentrated to give title compound (JJ-4) (13 g, 35.68 mmol, 94.34% yield) as a black oil, which was used in next step directly. LCMS (ES⁺): m/z 283.1 [M+H]⁺.

Synthesis of 2-[[5-cyclopropyl-3-(2,5-dichloropyrimidin-4-yl)pyrazol-1-yl]methoxy]ethyl-trimethyl-silane (JJ-5) To a solution of (5-cyclopropyl-1-((2-(trimethylsilyl)ethoxy)methyl)-1H-pyrazol-3-yl)boronic acid (JJ-4) (13 g, 35.68 mmol, 1 eq) in dioxane (60 mL) and H₂O (6 mL) was added 2,4,5-trichloropyrimidine (7.85 g, 42.81 mmol, 1.2 eq), Pd(dppf)Cl₂ (2.61 g, 3.57 mmol, 0.1 eq) and K₂CO₃ (14.79 g, 107.04 mmol, 3 eq), the mixture was stirred at 100 °C for 12 h under N₂. LCMS showed 2-[[5-cyclopropyl-3-(4,4,5,5-tetramethyl-1,3,2-dioxaborolan-2-yl)pyrazol-1-yl]methoxy]ethyl-trimethyl-silane (JJ-4) was consumed completely and the desired mass was detected. The reaction mixture was concentrated to give a residue. The residue was diluted with H₂O (30 mL) and extracted with EtOAc (30 mL*3). The combined organic layers were dried over Na₂SO₄, filtered and the filtrate was concentrated to give a residue. The residue was purified by flash silica gel chromatography (ISCO®; 40 g SepaFlash® Silica Flash Column, Eluent of 0~30% Ethyl acetate/Petroleum ethergradient @ 60 mL/min) to give title compound (JJ-5) (12 g, 31.14 mmol, 87.28% yield) as a colorless solid. LCMS (ES⁺): m/z 385.0 [M+H]⁺.

Synthesis of 2,5-dichloro-4-(5-cyclopropyl-1H-pyrazol-3-yl)pyrimidine (Compound JJ). To a solution of 2-[[5-cyclopropyl-3-(2,5-dichloropyrimidin-4-yl)pyrazol-1-yl]methoxy]ethyl-trimethyl-silane (JJ-5) (9 g, 23.35 mmol, 1 eq) in DCM (60 mL) was added TFA (13.31 g, 116.77 mmol, 8.67 mL, 5 eq). The mixture was stirred at 25 °C for 12 h. LCMS showed 2-[[5-cyclopropyl-3-(2,5-dichloropyrimidin-4-yl)pyrazol-1-yl]methoxy]ethyl-trimethyl-silane (JJ-5) was consumed completely and the desired mass was detected. The reaction mixture was concentrated to give a residue. The residue was triturated with Petroleum ether (10 mL). The resulting solid was collected by filtration, washed with Petroleum ether (10 mL) and dried to give title compound (Compound JJ) (2 g, 7.84 mmol, 33.57% yield) as a yellow oil. LCMS (ES⁺): m/z 255.0 [M+H]⁺.

### Synthesis of 5-(4-(2,5-dichloropyrimidin-4-yl)-1H-pyrazol-1-yl)pyridin-2(1H)-one (Compound KK)

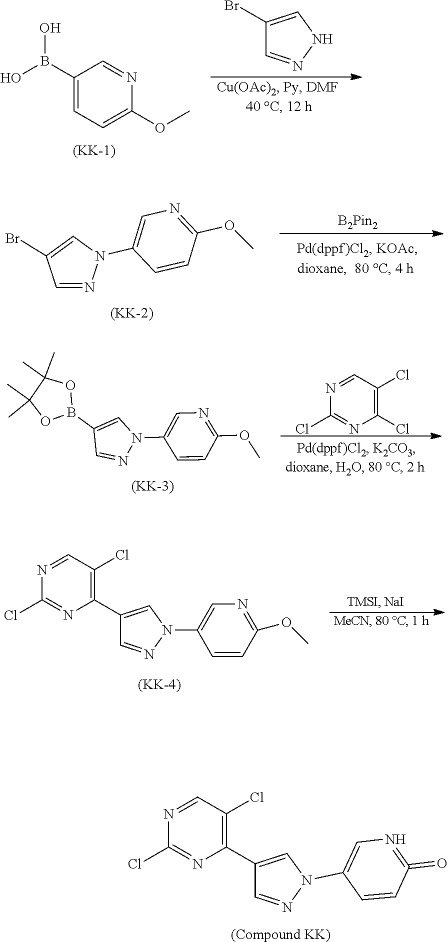

Synthesis of 5-(4-bromopyrazol-1-yl)-2-methoxy-pyridine (KK-2). To a solution of (6-methoxy-3-pyridyl) boronic acid (KK-1) (10 g, 65.38 mmol, 1 eq) in DMF (200 mL) was added 4-bromo-1H-pyrazole (10.57 g, 71.92 mmol, 1.1 eq), Cu(OAc)₂ (11.88 g, 65.38 mmol, 1 eq) and Py (15.52 g, 196.15 mmol, 15.83 mL, 3 eq). The mixture was stirred at 40 °C for 12 h. LCMS showed (6-methoxy-3-pyridyl) boronic acid (KK-1) was consumed completely and 88% product with desired mass was detected. The reaction mixture was diluted with water (100 mL) and the aqueous phase was extracted with DCM (50 mL*3). The combined organic layers were dried over Na₂SO₄, filtered and the filtrate was concentrated to give title compound (KK-2) (17 g, crude) as a white solid. LCMS (ES+): m/z 254.2 [M+H]+.

Synthesis of 2-methoxy-5-[4-(4, 4, 5, 5-tetramethyl-1, 3, 2-dioxaborolan-2-yl) pyrazol-1-yl]pyridine (KK-3). To a solution of 5-(4-bromopyrazol-1-yl)-2-methoxy-pyridine (KK-2) (3.5 g, 13.78 mmol, 1 eq) in dioxane (60 mL) was added 4,4,5,5-tetramethyl-2-(4,4,5,5-tetramethyl-1,3,2-dioxaborolan-2-yl)-1,3,2-dioxaborolane (7.00 g, 27.55 mmol, 2 eq), KOAc (4.06 g, 41.33 mmol, 3 eq) and Pd(dppf)Cl₂ (503.96 mg, 688.75 μmol, 0.05 eq). The mixture was stirred at 80 °C for 4 h under N₂. LCMS showed 5-(4-bromopyrazol-1-yl)-2-methoxy-pyridine (KK-2) was consumed completely and 47% product with desired mass was detected. The reaction mixture was filtered and the filter cake was washed with 60 mL of DCM, the filtrate concentrated to give title compound (KK-3) (5 g, crude) as a black oil. LCMS (ES+): m/z 302.3 [M+H]⁺.

Synthesis of 2, 5-dichloro-4-[1-(6-methoxy-3-pyridyl) pyrazol-4-yl]pyrimidine (KK-4). To a solution of 2-methoxy-5-[4-(4,4,5,5-tetramethyl-1,3,2-dioxaborolan-2-yl)pyrazol-1-yl]pyridine (KK-3) (4.15 g, 13.78 mmol, 1 eq) in dioxane (60 mL) and H₂O (6 mL) was added 2,4,5-trichloropyrimidine (2.53 g, 13.78 mmol, 1 eq), K₂CO₃ (5.71 g, 41.34 mmol, 3 eq) and Pd(dppf)Cl₂ (504.17 mg, 689.03 μmol, 0.05 eq). The mixture was stirred at 80 °C for 2 h under N₂. LCMS showed 2-methoxy-5-[4-(4,4,5,5-tetramethyl-1,3,2-dioxaborolan-2-yl)pyrazol-1-yl]pyridine (KK-3) was consumed completely and 33% product with desired mass was detected. The reaction mixture was diluted with water (200 mL) and the aqueous phase was extracted with DCM (30 mL*3). The combined organic layers were dried over Na₂SO₄, filtered and the filtrate was concentrated to give a residue. The residue was purified by flash silica gel chromatography (ISCO®; 40 g SepaFlash® Silica Flash Column, Eluent of 0~8% THF/Petroleum ethergradient @ 100 mL/min) to give title compound (KK-4) (3 g, 8.01 mmol, 58.12% yield, 86% purity) as a gray solid. LCMS (ES+): m/z 322.1 [M+H]⁺.

Synthesis of 5-(4-(2,5-dichloropyrimidin-4-yl)-1H-pyrazol-1-yl)pyridin-2(1H)-one (Compound KK). To a solution of 2,5-dichloro-4-[1-(6-methoxy-3-pyridyl)pyrazol-4-yl]pyrimidine (KK-4) (2 g, 6.21 mmol, 1 eq) in ACN (20 mL) was added NaI (4.65 g, 31.04 mmol, 5 eq) and TMSCl (3.37 g, 31.04 mmol, 3.94 mL, 5 eq). The mixture was stirred at 80 °C for 1 h. LCMS showed 2,5-dichloro-4-[1-(6-methoxy-3-pyridyl)pyrazol-4-yl]pyrimidine (KK-4) was consumed completely and 83% product with desired mass was detected. The reaction mixture was diluted with sat.aq.Na₂SO₄ (300 mL) and the aqueous phase was extracted with DCM (80 mL*3). The combined organic layers were dried over Na₂SO₄, filtered and the filtrate was concentrated to give a residue. The crude product was triturated with DCM/THF=3:1 (100 mL) to give title compound (Compound KK) (1.9 g, 5.36 mmol, 86.41% yield, 87% purity) as a white solid. LCMS (ES+): m/z 307.9 [M+H]⁺.

### Synthesis of 4-[4-(2,5-dichloropyrimidin-4-yl)pyrazol-1-yl]-1H-pyridin-2-one (Compound LL)

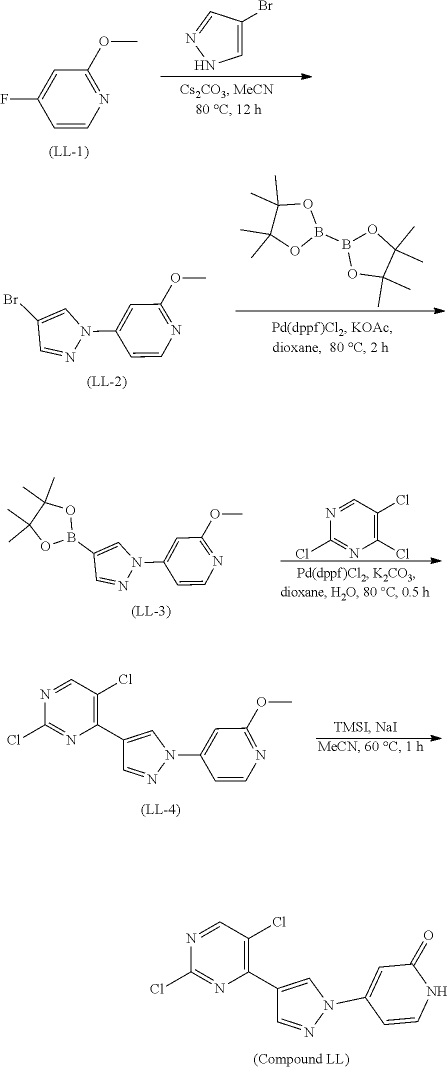

Synthesis of 4-(4-bromopyrazol-1-yl)-2-methoxy-pyridine (LL-2). To a solution of 4-fluoro-2-methoxy-pyridine (LL-1) (10 g, 78.67 mmol, 1 eq) in MeCN (200 mL) was added Cs₂CO₃ (76.89 g, 236.00 mmol, 3 eq) and 4-bromo-1H-pyrazole (12.72 g, 86.54 mmol, 1.1 eq). The mixture was stirred at 80 °C for 12 h. LCMS showed 4-fluoro-2-methoxy-pyridine (LL-1) was consumed completely and 95% product with desired mass was detected. The reaction was concentrated to give a residue. The reaction mixture was diluted with water (100 mL) and the aqueous phase was extracted with DCM (50 mL*3). The combined organic layers were dried over Na₂SO₄, filtered and the filtrate was concentrated to give a residue. The residue was purified by flash silica gel chromatography (ISCO®; 220 g SepaFlash® Silica Flash Column, Eluent of 0~5% Ethylacetate/Petroleum ethergradient @ 150 mL/min) to give title compound (LL-2) (18 g, 70.13 mmol, 89.15% yield, 99% purity) as a white solid. LCMS (ES+): m/z 254.1 [M+H]⁺.

Synthesis of 2-methoxy-4-[4-(4,4,5,5-tetramethyl-1,3,2-dioxaborolan-2-yl)pyrazol-1-yl]pyridine (LL-3). To a solution of 4-(4-bromopyrazol-1-yl)-2-methoxy-pyridine (LL-2) (7 g, 27.55 mmol, 1 eq) in dioxane (80 mL) was added 4,4,5,5-tetramethyl-2-(4,4,5,5-tetramethyl-1,3,2-dioxaborolan-2-yl)-1,3,2-dioxaborolane (13.99 g, 55.10 mmol, 2 eq), KOAc (8.11 g, 82.65 mmol, 3 eq) and Pd(dppf)Cl₂ (1.01 g, 1.38 mmol, 0.05 eq). The mixture was stirred at 80 °C for 2 h under N₂. LCMS showed 4-(4-bromopyrazol-1-yl)-2-methoxy-pyridine (LL-2) was consumed completely and 27% product with desired mass was detected. The reaction mixture was filtered and the filter cake was washed with 100 mL of DCM, the filtrate was concentrated to give title compound (LL-3) (8 g, crude) was obtained as a black oil. LCMS (ES+): m/z 302.3 [M+H]⁺.

Synthesis of 2,5-dichloro-4-[1-(2-methoxy-4-pyridyl)pyrazol-4-yl]pyrimidine (LL-4). To a solution of 2-methoxy-4-[4-(4,4,5,5-tetramethyl-1,3,2-dioxaborolan-2-yl)pyrazol-1-yl]pyridine (LL-3) (8 g, 26.57 mmol, 1 eq) in dioxane (120 mL) and H₂O (12 mL) was added K₂CO₃ (11.01 g, 79.70 mmol, 3 eq), 2,4,5-trichloropyrimidine (4.87 g, 26.57 mmol, 1 eq) and Pd(dppf)Cl₂ (971.89 mg, 1.33 mmol, 0.05 eq). The mixture was stirred at 80 °C for 0.5 hr under N₂. LCMS showed 2-methoxy-4-[4-(4,4,5,5-tetramethyl-1,3,2-dioxaborolan-2-yl)pyrazol-1-yl]pyridine (LL-3) was consumed completely and 45% product with desired mass was detected. The reaction mixture was diluted with water (200 mL) and the aqueous phase was extracted with DCM (80 mL*3). The combined organic layers were dried over Na₂SO₄, filtered and the filtrate was concentrated to give a residue. The residue was purified by flash silica gel chromatography (ISCO®; 80 g SepaFlash® Silica Flash Column, Eluent of 0-8% THF/Petroleum ethergradient @ 100 mL/min) to give title compound (LL-4) (4 g, 11.61 mmol, 43.70% yield, 93.5% purity) as a white solid. LCMS (ES+): m/z 321.9 [M+H]

Synthesis of 4-[4-(2,5-dichloropyrimidin-4-yl)pyrazol-1-yl]-1H-pyridin-2-one (Compound LL). To a solution of 2,5-dichloro-4-[1-(2-methoxy-4-pyridyl)pyrazol-4-yl]pyrimidine (LL-4) (3.5 g, 10.86 mmol, 1 eq) in MeCN (50 mL) was added NaI (8.14 g, 54.32 mmol, 5 eq) and TMSCl (5.90 g, 54.32 mmol, 6.89 mL, 5 eq). The mixture was stirred at 60 °C for 1 h. LCMS showed 2,5-dichloro-4-[1-(2-methoxy-4-pyridyl)pyrazol-4-yl]pyrimidine (LL-4) was consumed completely and 84% product with desired mass was detected. The reaction mixture was diluted with sat.aq.Na₂SO₄ (200 mL) and filtered, the filter cake was washed with 20 mL of water, dried filter cake in vacuum to give title compound (Compound LL) (2.9 g, crude) as a brown solid. LCMS (ES+): m/z 308.0 [M+H]

### Synthesis of 3-(4-(2,5-dichloropyrimidin-4-yl)-1H-pyrazol-1-yl)pyridin-2(1H)-one (Compound MM)

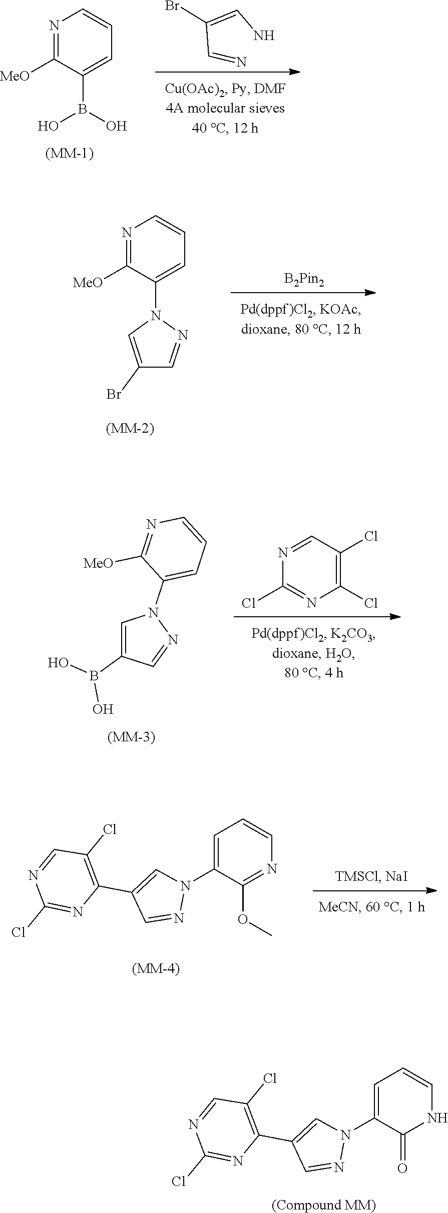

Synthesis of 3-(4-bromopyrazol-1-yl)-2-methoxy-pyridine (MM-2). To a solution of (2-methoxy-3-pyridyl)boronic acid (MM-1) (5 g, 32.69 mmol, 1 eq) in DMF (100 mL) was added 4-bromo-1H-pyrazole (4.80 g, 32.69 mmol, 1 eq), 4 A MOLECULAR SIEVE (5 g, 3.27 mmol), Py (7.76 g, 98.08 mmol, 7.92 mL, 3 eq) and Cu(OAc)₂ (5.94 g, 32.69 mmol, 1 eq). The mixture was stirred at 40 °C for 12 h under O₂. LCMS was showed (2-methoxy-3-pyridyl)boronic acid (MM-1) was consumed completely and the desired mass was detected. The reaction mixture was filtered and the filtrate was diluted with H₂O (50 mL) and extracted with DCM (50 mL*3). The combined organic layers were washed with brine (50 mL), and then dried over Na₂SO₄, filtered and the filtrate was concentrated to give a residue. The residue was purified by flash silica gel chromatography (ISCO®; 20 g SepaFlash®Silica Flash Column, Eluent of 0~100% Ethyl acetate/Petroleum ethergradient @ 100 mL/min) to give title compound (MM-2) (2.4 g, 9.45 mmol, 9.63% yield) was obtained as a white solid. LCMS (ES+): m/z 254.0 [M+H]⁺.

Synthesis of 2-methoxy-3-[4-(4,4,5,5-tetramethyl-1,3,2-dioxaborolan-2-yl)pyrazol-1-yl]pyridine (MM-3). To a solution of 3-(4-bromopyrazol-1-yl)-2-methoxy-pyridine (MM-2) (3.1 g, 12.20 mmol, 1 eq) in dioxane (50 mL) was added 4,4,5,5-tetramethyl-2-(4,4,5,5-tetramethyl-1,3,2-dioxaborolan-2-yl)-1,3,2-dioxaborolane (3.10 g, 12.20 mmol, 1 eq), KOAc (3.59 g, 36.60 mmol, 3 eq) and Pd(dppf)Cl₂ (446.37 mg, 610.04 μmol, 0.05 eq) with N₂. The reaction stirred at 80 °C for 12 h under N₂. LCMS showed 3-(4-bromopyrazol-1-yl)-2-methoxy-pyridine (MM-2) was consumed completely and desired mass was detected. After filtration, vacuum distillation removes solvents to give title compound (MM-3) (3.7 g, crude) as a black solid. LCMS (ES+): m/z 220.1 [M+H]⁺.

Synthesis of 2,5-dichloro-4-[1-(2-methoxy-3-pyridyl) pyrazol-4-yl]pyrimidine. (MM-4). To a solution of 2-methoxy-3-[4-(4,4,5,5-tetramethyl-1,3,2-dioxaborolan-2-yl)pyrazol-1-yl]pyridine (MM-3) (3.4 g, 11.29 mmol, 1 eq) in dioxane (30 mL) and H₂O (3 mL) was added 2,4,5-trichloropyrimidine (2.28 g, 12.42 mmol, 1.1 eq), Pd(dppf)Cl₂ (413.06 mg, 564.51 μmol, 0.05 eq) and K₂CO₃ (4.68 g, 33.87 mmol, 3 eq) under N₂. The mixture was stirred at 80 °C for 4 h. LCMS showed 2-methoxy-3-[4-(4,4,5,5-tetramethyl-1,3,2-dioxaborolan-2-yl)pyrazol-1-yl]pyridine (MM-3) was consumed completely and the desired mass was detected. The reaction was filtered and the filtrate was concentrated under reduced pressure to give a residue. The residue was purified by flash silica gel chromatography (40 g Silica Flash Column, Eluent of 0~100% Ethyl acetate/Petroleum ethergradient @ 60 mL/min) to give compound (MM-4) (2.4 g, 7.11 mmol, 62.98% yield, 95.45% purity) as a white solid. LCMS (ES+): m/z 322.1 [M+H]⁺.

Synthesis of 3-(4-(2,5-dichloropyrimidin-4-yl)-1H-pyrazol-1-yl)pyridin-2(1H)-one (Compound MM). To a solution of NaI (6.05 g, 40.35 mmol, 5 eq) in MeCN (150 mL) was added 2,5-dichloro-4-[1-(2-methoxy-3-pyridyl) pyrazol-4-yl]pyrimidine (MM-4) (2.6 g, 8.07 mmol, 1 eq) and TMSCl (4.38 g, 40.35 mmol, 5.12 mL, 5 eq). The mixture was stirred at 60 °C for 1 h. LCMS showed 2,5-dichloro-4-[1-(2-methoxy-3-pyridyl) pyrazol-4-yl]pyrimidine (MM-4) was consumed completely and 47% product with desired mass was detected. Vacuum distillation removes solvents to obtain crude products. The crude products were added to H₂O (40 mL), and extracted with EtOAc (40 mL*3). The combined organic layers were washed with brine (20 mL), dried over Na₂SO₄, filtered and the filtrate was concentrated under reduced pressure to give a residue. The residue was purified by flash silica gel chromatography (ISCO; 40 g Silica Flash Column, Eluent of 0~100% Ethyl acetate/Petroleum ethergradient @ 60 mL/min) to give compound (Compound MM) (1.6 g, 4.67 mmol, 57.91% yield, 90% purity) as a yellow solid. LCMS (ES+): m/z 308.1 [M+H]⁺.

### Synthesis of 2,5-dichloro-4-[1-(6-methoxy-2-pyridyl)pyrazol-4-yl]pyrimidine (Compound NN)

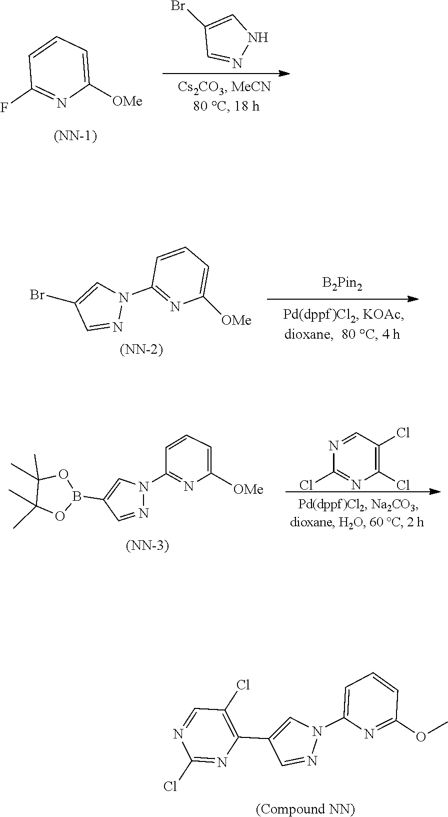

Synthesis of 2-(4-bromopyrazol-1-yl)-6-methoxy-pyridine (NN-2). To a solution of 2-fluoro-6-methoxy-pyridine (NN-1) (10 g, 78.67 mmol, 1 eq) in MeCN (500 mL) was added Cs₂CO₃ (128.16 g, 393.34 mmol, 5 eq) followed by 4-bromo-1H-pyrazole (23.12 g, 157.34 mmol, 2 eq) at 25 °C. The reaction mixture was stirred at 80 °C for 18 h. LCMS showed 2-fluoro-6-methoxy-pyridine (NN-1) was remaining and desired mass was detected. The reaction mixture was filtered to remove cesium salts and then the filtrate was evaporated to give title compound (NN-2) (19.99 g, 78.68 mmol, 100.00% yield) as a white solid. LCMS (ES+): m/z 254.0 [M+H]⁺.

Synthesis of 2-methoxy-6-[4-(4,4,5,5-tetramethyl-1,3,2-dioxaborolan-2-yl)pyrazol-1-yl]pyridine (NN-3). To a solution of 2-(4-bromopyrazol-1-yl)-6-methoxy-pyridine (NN-2) (5 g, 19.68 mmol, 1 eq) and 4,4,5,5-tetramethyl-2-(4,4,5,5-tetramethyl-1,3,2-dioxaborolan-2-yl)-1,3,2-dioxaborolane (6.00 g, 23.61 mmol, 1.2 eq) in dioxane (50 mL) was slowly added Pd(dppf)Cl₂ (1.44 g, 1.97 mmol, 0.1 eq) and KOAc (7.73 g, 78.71 mmol, 4 eq) at 25 °C. The reaction mixture was stirred at 80 °C for 4 h under N₂. LCMS showed 2-(4-bromopyrazol-1-yl)-6-methoxy-pyridine (NN-2) was consumed completely and desired mass was detected. The reaction mixture was concentrated under reduced pressure to remove give title compound (NN-3) (5.93 g, crude) as a black oil and used into the next step without further purification. LCMS (ES+): m/z 302.0 [M+H]⁺.

Synthesis of 2,5-dichloro-4-[1-(6-methoxy-2-pyridyl)pyrazol-4-yl]pyrimidine (Compound NN). To a solution of 2,4,5-trichloropyrimidine (3.61 g, 19.69 mmol, 1 eq) and 2-methoxy-6-[4-(4,4,5,5-tetramethyl-1,3,2-dioxaborolan-2-yl)pyrazol-1-yl]pyridine (NN-3) (5.93 g, 19.69 mmol, 1 eq) in dioxane (200 mL) and H₂O (20 mL) was added Pd(dppf)Cl₂ (1.44 g, 1.97 mmol, 0.1 eq) and Na₂CO₃ (6.26 g, 59.07 mmol, 3 eq) at 25 °C. The reaction mixture was stirred at 60 °C for 2 h under N₂. LCMS showed 2,4,5-trichloropyrimidine (NN-3) was consumed completely and desired mass was detected. The residue was diluted with H₂O (200 mL) and extracted with EtOAc (200 mL*3). The combined organic phase was washed with brine (100 mL), dried over anhydrous Na₂SO₄, filtered and concentrated give a residue. The residue was purified by flash silica gel chromatography (ISCO®; 40 g SepaFlash® Silica Flash Column, Eluent of 0~10% Ethyl acetate/Petroleum ethergradient @ 80 L/min) to give title compound (Compound NN) (5 g, 15.52 mmol, 78.82% yield) as a white solid. LCMS (ES+): m/z 322.0 [M+H]⁺.

### Synthesis of 2, 5-dichloro-4-[1-(4-fluorophenyl) pyrazol-4-yl]pyrimidine (Compound OO)

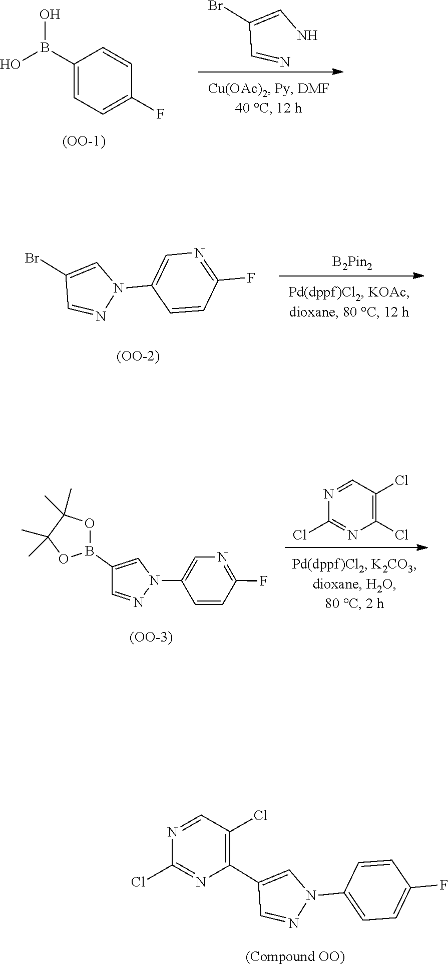

Synthesis of 5-(4-bromo-1H-pyrazol-1-yl)-2-fluoropyridine (OO-2). To a solution of (4-fluorophenyl)boronic acid (OO-1) (5 g, 35.73 mmol, 1 eq) in DMF (100 mL) was added 4-bromo-1H-pyrazole (5.78 g, 39.31 mmol, 1.1 eq), Cu(OAc)₂ (6.49 g, 35.73 mmol, 1 eq) and Py (8.48 g, 107.20 mmol, 8.65 mL, 3 eq). The mixture was stirred at 40 °C for 12 h under 02. LCMS showed (4-fluorophenyl)boronic acid (OO-1) was consumed completely and 87% product with desired mass was detected. The reaction mixture was diluted with water (100 mL) and the aqueous phase was extracted with DCM (30 mL*3). The combined organic layers were dried over Na₂SO₄, filtered and the filtrate was concentrated to give a residue. The residue was purified by flash silica gel chromatography (ISCO®; 80 g SepaFlash® Silica Flash Column, Eluent of 0~5% Ethyl acetate/Petroleum ethergradient @ 100 mL/min) to give title compound (OO-2) (8 g, 32.52 mmol, 91.01% yield, 98% purity) as a white solid. LCMS (ES+): m/z 241.9 [M+H]⁺.

Synthesis of 2-fluoro-5-(4-(4,4,5,5-tetramethyl-1,3,2-dioxaborolan-2-yl)-1H-pyrazol-1-yl)pyridine (OO-3). To a solution of 5-(4-bromo-1H-pyrazol-1-yl)-2-fluoropyridine (OO-2) (6 g, 24.89 mmol, 1 eq) in dioxane (100 mL) and was added 4,4,5,5-tetramethyl-2-(4,4,5,5-tetramethyl-1,3,2-dioxaborolan-2-yl)-1,3,2-dioxaborolane (12.64 g, 49.78 mmol, 2 eq), KOAc (7.33 g, 74.67 mmol, 3 eq) and Pd(dppf)Cl₂ (910.61 mg, 1.24 mmol, 0.05 eq). The mixture was stirred at 80 °C for 12 h under N₂. LCMS showed 5-(4-bromo-1H-pyrazol-1-yl)-2-fluoropyridine (OO-2) was consumed completely and 45% product with desired mass was detected. The reaction mixture was filtered and the filter cake was washed with 50 mL of DCM, the filtrate concentrated to give title compound (OO-3) (6 g, crude) as a black oil. LCMS (ES+): m/z 290.1 [M+H]⁺.

Synthesis of 2,5-dichloro-4-[1-(4-fluorophenyl) pyrazol-4-yl]pyrimidine (Compound 00). To a solution of 2-fluoro-5-(4-(4,4,5,5-tetramethyl-1,3,2-dioxaborolan-2-yl)-1H-pyrazol-1-yl)pyridine (OO-3) (7.17 g, 24.89 mmol, 1 eq) in dioxane (100 mL) and H₂O (10 mL) was added 2,4,5-trichloropyrimidine (3.20 g, 17.42 mmol, 0.7 eq), K₂CO₃ (10.32 g, 74.66 mmol, 3 eq) and Pd(dppf)Cl₂ (910.43 mg, 1.24 mmol, 0.05 eq). The mixture was stirred at 80 °C for 2 h under N₂. LCMS showed 2-fluoro-5-(4-(4,4,5,5-tetramethyl-1,3,2-dioxaborolan-2-yl)-1H-pyrazol-1-yl)pyridine (OO-3) was consumed completely and 20% product with desired mass was detected. The reaction mixture was diluted with water (400 mL) and the aqueous phase was extracted with DCM (40 mL×3). The combined organic layers were dried over Na₂SO₄, filtered and the filtrate was concentrated to give a residue. The residue was purified by flash silica gel chromatography (ISCO®; 80 g SepaFlash® Silica Flash Column, Eluent of 0~5% THF/Petroleum ethergradient @ 100 mL/min) to give title compound (Compound 00) (1.9 g, 5.53 mmol, 22.23% yield, 90% purity) as a white solid. LCMS (ES+): m/z 309.1 [M+H]⁺.

### Example 2. Synthesis of 4-((4-([1,1′-biphenyl]-3-yl)-5-chloropyrimidin-2-yl)amino)cyclohexane-1-carboxamide (Compound 1)

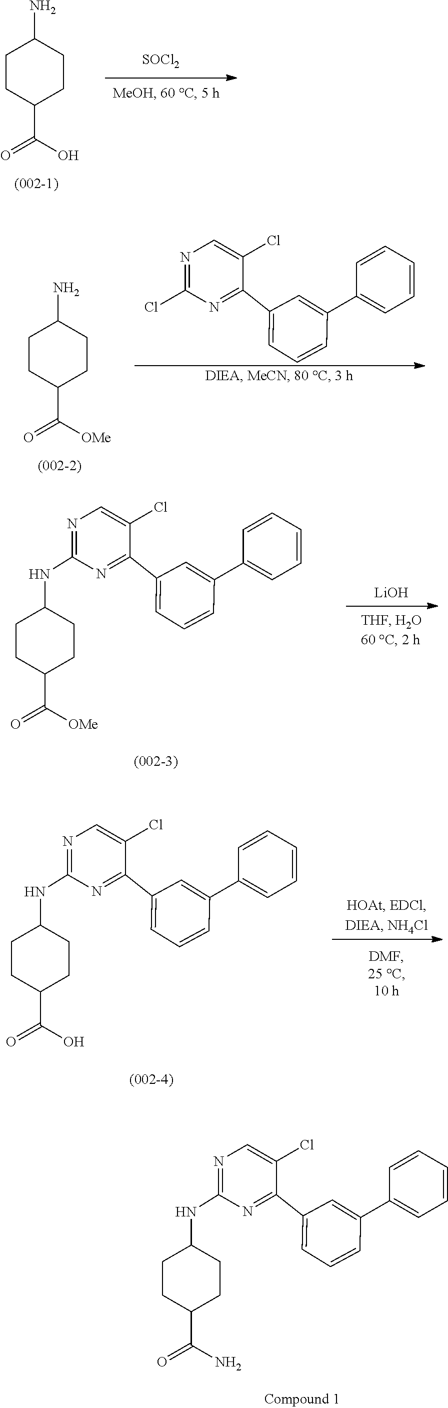

Synthesis of methyl 4-aminocyclohexane-1-carboxylate (002-2). To a solution of 4-aminocyclohexane-1-carboxylic acid (002-1) (5.00 g, 34.9 mmol, 1.00 eq) in MeOH (120 mL) was added SOCl₂ (8.31 g, 69.8 mmol, 5.07 mL, 2.00 eq). The mixture was stirred at 60 °C for 5 h. LCMS showed no desired mass was detected. The reaction was monitored by TLC. TLC (Petroleum ether:Ethyl acetate=0:1) indicated 4-aminocyclohexane-1-carboxylic acid (002-1) was consumed completely and one new spot formed. The reaction mixture was concentrated to give the title compound (002-2) (5.00 g, 31.8 mmol, 91.1% yield) as a yellow solid. LCMS (ES+): m/z 158.1 [M+H]⁺.

Synthesis of methyl 4-((4-([1,1′-biphenyl]-3-yl)-5-chloropyrimidin-2-yl)amino)cyclohexane-1-carboxylate (002-3). To a solution of methyl 4-aminocyclohexane-1-carboxylate (002-2) (1.00 g, 6.36 mmol, 1.10 eq) and 2,5-dichloro-4-(3-phenylphenyl)pyrimidine (Compound A) (1.74 g, 5.78 mmol, 1.00 eq) in MeCN (20 mL) was added DIEA (747 mg, 5.78 mmol, 1.01 mL, 1.00 eq). The mixture was stirred at 80 °C for 3 h. LCMS showed methyl 4-aminocyclohexane-1-carboxylate (002-2) was consumed completely and desired mass was detected. The reaction mixture was concentrated to give the title compound (002-3) (2.40 g, 5.69 mmol, 98.4% yield) as a yellow solid. LCMS (ES+): m/z 420.3 [M−H]+.

Synthesis of 4-((4-([1,1′-biphenyl]-3-yl)-5-chloropyrimidin-2-yl)amino)cyclohexane-1-carboxylic acid (002-4). To a solution of methyl 4-((4-([1,1′-biphenyl]-3-yl)-5-chloropyrimidin-2-yl)amino)cyclohexane-1-carboxylate (002-3) (2.40 g, 5.69 mmol, 1.00 eq) in THF (21 mL) and H₂O (7 mL) was added LiOH (409 mg, 17.1 mmol, 3.00 eq). The mixture was stirred at 60 °C for 2 h. LCMS showed methyl 4-((4-([1,1′-biphenyl]-3-yl)-5-chloropyrimidin-2-yl)amino)cyclohexane-1-carboxylate (002-3) was consumed completely and desired mass was detected. The reaction mixture was concentrated. The residue was purified by prep-HPLC (column: Phenomenex C18 80*40 mm*3 μm; mobile phase: [water (NH₄HCO₃)-ACN]; B %: 30%-60%, 8 min) to give the title compound (002-4) (130 mg, 319 μmol, 5.60% yield) as a white solid. LCMS (ES−): m/z 406.1 [M−H]−.

Synthesis of 4-((4-([1,1′-biphenyl]-3-yl)-5-chloropyrimidin-2-yl)amino)cyclohexane-1-carboxamide (Compound 1). To a solution of 4-((4-([1,1′-biphenyl]-3-yl)-5-chloropyrimidin-2-yl)amino)cyclohexane-1-carboxylic acid (002-4) (130 mg, 319 μmol, 1.00 eq), HOAt (52.1 mg, 382 μmol, 53.5 μL, 1.20 eq) and EDCI (73.3 mg, 382 μmol, 1.20 eq) in DMF (3 mL) were added DIEA (124 mg, 956 μmol, 166 μL, 3.00 eq) and NH₄Cl (34.1 mg, 637 μmol, 2.00 eq). The mixture was stirred at 25 °C for 10 h. LCMS showed 4-((4-([1,1′-biphenyl]-3-yl)-5-chloropyrimidin-2-yl)amino)cyclohexane-1-carboxylic acid (002-4) was consumed completely and desired mass was detected. The reaction mixture was concentrated. The residue was purified by prep-HPLC (column: Phenomenex C18 75*30 mm*3 μm; mobile phase: [water (NH₄HCO₃)-ACN]; B %: 50%-80%, 8 min) to give the title compound (Compound 1) (80.5 mg, 198 μmol, 62.1% yield, 100% purity) as a white solid. LCMS (ES−): m/z 405.2 [M−H]−.

### Example 3. Synthesis of (4-((4-([1,1′-biphenyl]-3-yl)-5-chloropyrimidin-2-yl)amino)piperidin-1-yl)(piperidin-4-yl)methanone (Compound 2)

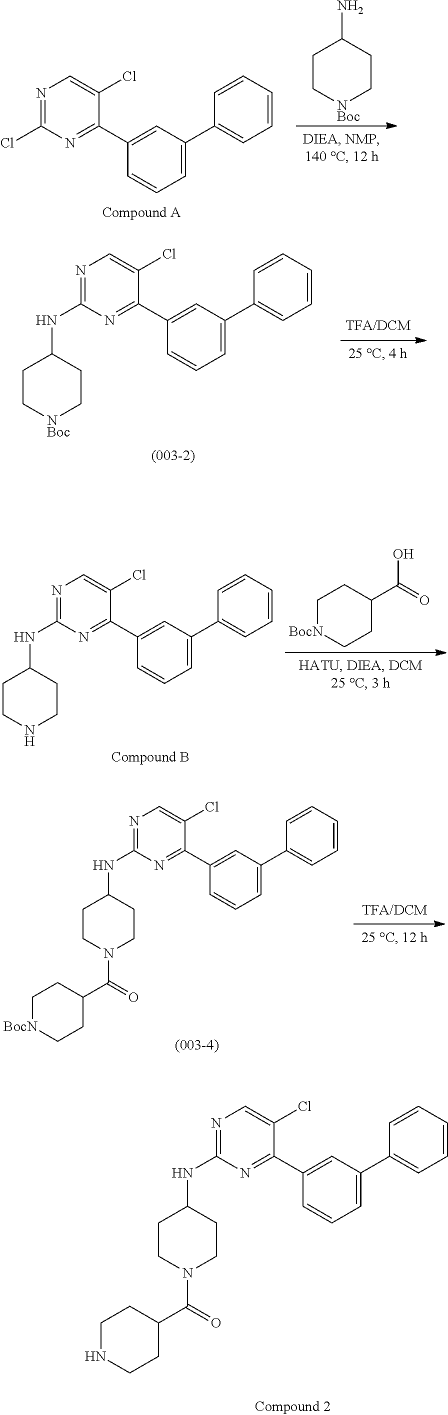

Synthesis of tert-butyl 4-((4-([1,1′-biphenyl]-3-yl)-5-chloropyrimidin-2-yl)amino)piperidine-1-carboxylate (003-2). To a solution of tert-butyl 4-aminopiperidine-1-carboxylate (10.0 g, 49.9 mmol, 1.00 eq) in NMP (200 mL) and DIEA (16 mL) was added 4-([1,1′-biphenyl]-3-yl)-2,5-dichloropyrimidine (Compound A) (003-1) (18.1 g, 59.9 mmol, 1.20 eq) at 20 °C. The mixture was stirred at 140 °C for 12 h. LCMS showed 4-([1,1′-biphenyl]-3-yl)-2,5-dichloropyrimidine (Compound A) (003-1) was consumed completely and the desired mass was detected. Water (150 mL) was added to the reaction, the aqueous phase was extracted with EtOAc (150 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 5:1) to give the title compound (003-2) (5.51 g, 11.9 mmol, 23.7% yield) as a white solid. LCMS (ES+): m/z 409.1 [M+H−56]+.

Synthesis of 4-([1,1′-biphenyl]-3-yl)-5-chloro-N-(piperidin-4-yl)pyrimidin-2-amine (Compound B). A solution of tert-butyl 4-((4-([1,1′-biphenyl]-3-yl)-5-chloropyrimidin-2-yl)amino)piperidine-1-carboxylate (003-2) (2.00 g, 4.30 mmol, 1.00 eq) in TFA (1 mL) and DCM (5 mL) was stirred at 25 °C for 4 h. LCMS showed 4-((4-([1,1′-biphenyl]-3-yl)-5-chloropyrimidin-2-yl)amino)piperidine-1-carboxylate (003-2) was consumed completely and the desired mass was detected. The reaction mixture was concentrated under reduced pressure to give the title compound (003-3) (4.20 g, crude, TFA) as a yellow oil. LCMS (ES+): m/z 365.2 [M+H]+.

Synthesis of tert-butyl 4-(4-((4-([1,1′-biphenyl]-3-yl)-5-chloropyrimidin-2-yl)amino)piperidine-1-carbonyl)piperidine-1-carboxylate (003-4). To a solution of 4-([1,1′-biphenyl]-3-yl)-5-chloro-N-(piperidin-4-yl)pyrimidin-2-amine (Compound B) (1.20 g, 2.51 mmol, 1.00 eq, TFA) in DCM (6 mL) were added 1-tert-butoxycarbonylpiperidine-4-carboxylic acid (689 mg, 3.01 mmol, 1.20 eq), HATU (1.43 g, 3.76 mmol, 1.50 eq) and DIEA (1.62 g, 12.5 mmol, 2.18 mL, 5.00 eq) at 25 °C. The mixture was stirred at 25 °C for 3 h. LC-MS showed 4-([1,1′-biphenyl]-3-yl)-5-chloro-N-(piperidin-4-yl)pyrimidin-2-amine (Compound B) was consumed completely and the desired mass was detected. Water (20 mL) was added to reaction, the aqueous phase was extracted with EtOAc (15 mL*3), the combined organic layers were dried over Na₂SO₄, filtered and the filtrate was concentrated under reduced pressure. The residue was purified by prep-TLC (SiO₂, Petroleum ether:Ethyl acetate=1:3) to give the title compound (003-4) (580 mg, 1.01 mmol, 40.2% yield) as a yellow oil. LCMS (ES+): m/z 576.4 [M+H]+.

Synthesis of (4-((4-([1,1′-biphenyl]-3-yl)-5-chloropyrimidin-2-yl)amino)piperidin-1-yl)(piperidin-4-yl)methanone (Compound 2). A solution of tert-butyl 4-(4-((4-([1,1-biphenyl]-3-yl)-5-chloropyrimidin-2-yl)amino)piperidine-1-carbonyl)piperidine-1-carboxylate (003-4) (200 mg, 347 μmol, 1.00 eq) in TFA (1 mL) and DCM (5 mL) was stirred at 25 °C for 12 h. LCMS showed tert-butyl 4-(4-((4-([1,1′-biphenyl]-3-yl)-5-chloropyrimidin-2-yl)amino)piperidine-1-carbonyl)piperidine-1-carboxylate (003-4) was consumed completely and the desired mass was detected. The reaction mixture was concentrated under reduced pressure. The residue was purified by prep-HPLC (TFA column: Phenomenex luna C18 80*40 mm*3 μm; mobile phase: [water (FA)-ACN]; B %: 10%-50%, 8 min) to give the title compound (Compound 2) (75.6 mg, 159 μmol, 45.8% yield, 100% purity) as a white solid. LCMS (ES+): m/z 476.2 [M+H]+.

### Example 4. Synthesis of (4-((4-([1,1′-biphenyl]-3-yl)-5-chloropyrimidin-2-yl)amino)piperidin-1-yl)(1-methylpiperidin-4-yl)methanone (Compound 3)

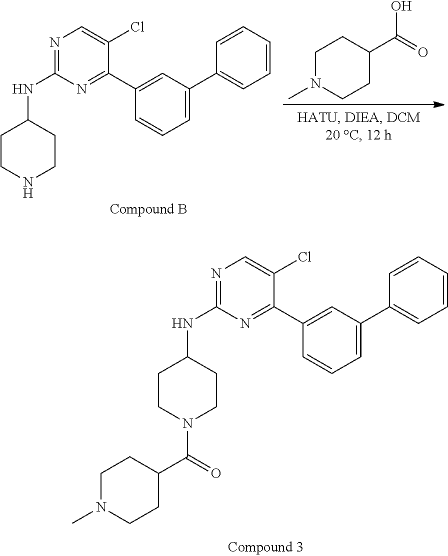

Synthesis of (4-((4-([1,1′-biphenyl]-3-yl)-5-chloropyrimidin-2-yl)amino)piperidin-1-yl)(1-methylpiperidin-4-yl)methanone (Compound 3). To a solution of 4-([1,1′-biphenyl]-3-yl)-5-chloro-N-(piperidin-4-yl)pyrimidin-2-amine (Compound B) (004-1) (200 mg, 418 μmol, 1.00 eq, TFA) and 1-methylpiperidine-4-carboxylic acid (71.8 mg, 501 μmol, 1.20 eq) in DCM (5 mL) were added HATU (238 mg, 626 μmol, 1.50 eq) and DIEA (270 mg, 2.09 mmol, 364 μL, 5.00 eq) at 20 °C. The resulting mixture was stirred at 20 °C for 12 h. LCMS showed 4-([1,1′-biphenyl]-3-yl)-5-chloro-N-(piperidin-4-yl)pyrimidin-2-amine (Compound B) (004-1) was consumed and the desired mass was detected. The mixture was concentrated in vacuo. The mixture was purified by prep-HPLC (column: Waters Xbridge Prep OBD C18 150*40 mm*10 μm; mobile phase: [water (NH₄HCO₃)-ACN]; B %: 55%-95%, 8 min) to give the title compound (Compound 3) (128 mg, 262 μmol, 62.5% yield, 100% purity) as a white solid. LCMS (ES+): m/z 490.2 [M+H]+.

### Example 5. Synthesis of 1-(4-(4-((4-([1,1′-biphenyl]-3-yl)-5-chloropyrimidin-2-yl)amino)piperidine-1-carbonyl)piperidin-1-yl)ethan-1-one (Compound 4)

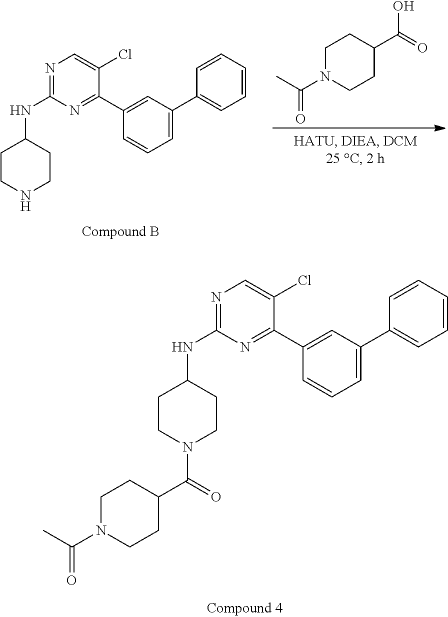

Synthesis of 1-(4-(4-((4-([1,1′-biphenyl]-3-yl)-5-chloropyrimidin-2-yl)amino)piperidine-1-carbonyl)piperidin-1-yl)ethan-1-one (Compound 4). To a solution of 1-acetylpiperidine-4-carboxylic acid (85.8 mg, 501 μmol, 1.20 eq) in DCM (3 mL) were added HATU (238 mg, 626 μmol, 1.50 eq) and DIEA (270 mg, 2.09 mmol, 364 μL, 5.00 eq) at 25 °C, then 4-([1,1′-biphenyl]-3-yl)-5-chloro-N-(piperidin-4-yl)pyrimidin-2-amine (Compound B) (005-1) (200 mg, 418 μmol, 1.00 eq, TFA) was added to the mixture, the mixture was stirred at 25 °C for 2 h. LCMS showed 4-([1,1′-biphenyl]-3-yl)-5-chloro-N-(piperidin-4-yl)pyrimidin-2-amine (Compound B) was consumed, desired target MS was detected. The mixture was concentrated in vacuo. The residue was purified by prep-HPLC (column: Phenomenex luna C18 80*40 mm*3 μm; mobile phase: [water (FA)-ACN]; B %: 40%-80%, 8 min) to give the title compound (Compound 4) (116 mg, 225 μmol, 53.8% yield, 100% purity) as a white solid. LCMS (ES+): m/z 518.1 [M+H]+.

### Example 6. Synthesis of 1-(4-((4-([1,1′-biphenyl]-3-yl)-5-chloropyrimidin-2-yl)amino)piperidin-1-yl)-2-(piperidin-4-yl)ethan-1-one (Compound 5)

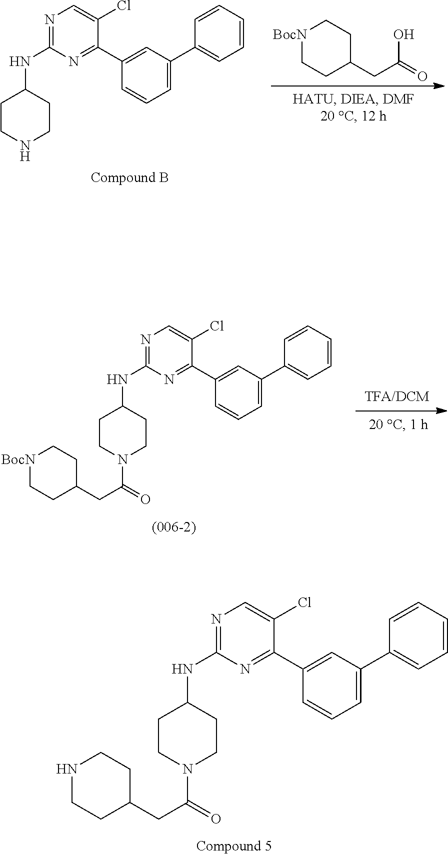 [SMILES: Clc1cnc(NC2CCNCC2)nc1-c1cccc(-c2ccccc2)c1 ; CC(C)(C)OC(=O)N1CCC(CC(=O)O)CC1 ; CC(C)(C)OC(=O)N1CCC(CC(=O)N2CCC(Nc3ncc(Cl)c(-c4cccc(-c5ccccc5)c4)n3)CC2)CC1 ; O=C(CC1CCNCC1)N1CCC(Nc2ncc(Cl)c(-c3cccc(-c4ccccc4)c3)n2)CC1]

Synthesis of tert-butyl4-(2-(4-((4-([1,1′-biphenyl]-3-yl)-5-chloropyrimidin-2-yl)amino)piperidin-1-yl)-2-oxoethyl)piperidine-1-carboxylate (006-2). To a stirred solution of 2-(1-(tert-butoxycarbonyl)piperidin-4-yl)acetic acid (267 mg, 1.10 mmol, 1.00 eq) in DMF (3 mL) were added HATU (459 mg, 1.21 mmol, 1.10 eq) and DIEA (439 mg, 3.40 mmol, 592 L, 3.10 eq), then added 4-([1,1′-biphenyl]-3-yl)-5-chloro-N-(piperidin-4-yl)pyrimidin-2-amine (Compound B) (400 mg, 1.10 mmol, 1.00 eq) at 20 °C. The resulting mixture was stirred at 20 °C for 12 h. LCMS showed that 4-([1,1′-biphenyl]-3-yl)-5-chloro-N-(piperidin-4-yl)pyrimidin-2-amine (Compound B) was consumed and the desired mass was detected. The mixture was diluted with H₂O (5 mL) and then extracted with EtOAc (5 mL*3). The combined organic layers were washed with saturated aqueous NaCl solution (5 mL*3), dried over Na₂SO₄, filtered and the filtrate was concentrated in vacuo to give the title compound (006-2) (650 mg, crude) as a yellow oil. LCMS (ES+): m/z 590.3 [M+H]+.

Synthesis of 1-(4-((4-([1,1′-biphenyl]-3-yl)-5-chloropyrimidin-2-yl)amino)piperidin-1-yl)-2-(piperidin-4-yl)ethan-1-one (Compound 5). A solution of tert-butyl4-(2-(4-((4-([1,1′-biphenyl]-3-yl)-5-chloropyrimidin-2-yl)amino)piperidin-1-yl)-2-oxoethyl)piperidine-1-carboxylate (006-2) (300 mg, 508 μmol, 1.00 eq) in TFA (1 mL) and DCM (3 mL) was stirred at 20 °C for 1 h. LCMS showed that tert-butyl4-(2-(4-((4-([1,1′-biphenyl]-3-yl)-5-chloropyrimidin-2-yl)amino)piperidin-1-yl)-2-oxoethyl)piperidine-1-carboxylate (006-2) was consumed and the desired mass was detected. The mixture was purified by acidic prep-HPLC (column: Phenomenex luna C18 80*40 mm*3 μm; mobile phase: [water (FA)-ACN]; B %: 25%-65%, 8 min) to give the title compound (Compound 5) (113 mg, 230 μmol, 45.3% yield, 100% purity) as a yellow solid. LCMS (ES+): m/z 490.2 [M+H]+.

### Example 7. Synthesis of 1-(4-((4-([1,1′-biphenyl]-3-yl)-5-chloropyrimidin-2-yl)amino)piperidin-1-yl)-2-(1-acetylpiperidin-4-yl)ethan-1-one (Compound 6)

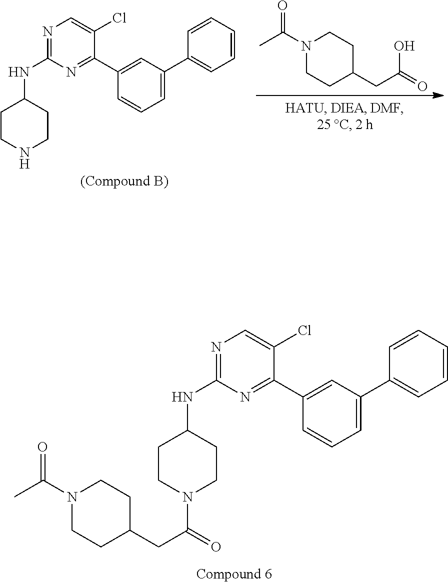

Synthesis of 1-(4-((4-([1,1′-biphenyl]-3-yl)-5-chloropyrimidin-2-yl)amino)piperidin-1-yl)-2-(1-acetylpiperidin-4-yl)ethan-1-one (Compound 6). To a solution of 2-(1-acetylpiperidin-4-yl)acetic acid (92.8 mg, 501 μmol, 1.20 eq) and 4-([1,1′-biphenyl]-3-yl)-5-chloro-N-(piperidin-4-yl)pyrimidin-2-amine (Compound B) (200 mg, 418 μmol, 1.00 eq, TFA) in DCM (3 mL) were added HATU (238 mg, 626 μmol, 1.50 eq) and DIEA (270 mg, 2.09 mmol, 364 μL, 5.00 eq) at 25 °C, the mixture was stirred at 25 °C for 2 h. LCMS showed 4-([1,1′-biphenyl]-3-yl)-5-chloro-N-(piperidin-4-yl)pyrimidin-2-amine (Compound B) was consumed, desired target MS was detected. The organic layer was filtered and the filtrate was concentrated under reduced pressure to give a residue. The residue was purified by prep-HPLC (column: Phenomenex Luna C18 200*40 mm*10 μm; mobile phase: [water (FA)-ACN]; B %: 50%-80%, 8 min) to give the title compound (Compound 6) (132 mg, 248 mol, 59.4% yield, 100% purity) as a white solid. LCMS (ES+): m/z 532.1 [M+H]+.

### Example 8. Synthesis of 1-(4-((4-([1,1′-biphenyl]-3-yl)-5-chloropyrimidin-2-yl)amino)piperidin-1-yl)-3-(piperidin-4-yl)propan-1-one (Compound 7)

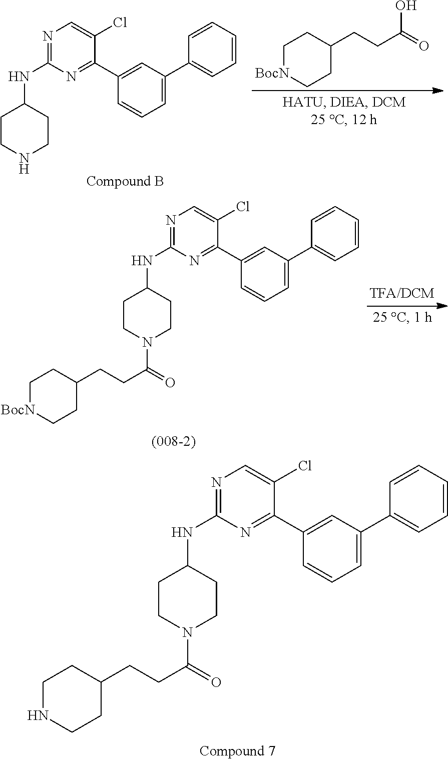 [SMILES: Clc1cnc(NC2CCNCC2)nc1-c1cccc(-c2ccccc2)c1 ; CC(C)(C)OC(=O)N1CCC(CCC(=O)O)CC1 ; CC(C)(C)OC(=O)N1CCC(CCC(=O)N2CCC(Nc3ncc(Cl)c(-c4cccc(-c5ccccc5)c4)n3)CC2)CC1 ; O=C(CCC1CCNCC1)N1CCC(Nc2ncc(Cl)c(-c3cccc(-c4ccccc4)c3)n2)CC1]

Synthesis of tert-butyl 4-(3-(4-((4-([1,1′-biphenyl]-3-yl)-5-chloropyrimidin-2-yl)amino)piperidin-1-yl)-3-oxopropyl)piperidine-1-carboxylate (008-2). To a solution of 4-([1,1′-biphenyl]-3-yl)-5-chloro-N-(piperidin-4-yl)pyrimidin-2-amine (Compound B) (600 mg, 1.64 mmol, 1.00 eq) in DCM (12 mL) were added 3-(1-tert-butoxycarbonyl-4-piperidyl)propanoic acid (508 mg, 1.97 mmol, 1.20 eq), HATU (938 mg, 2.47 mmol, 1.50 eq) and DIEA (1.06 g, 8.22 mmol, 1.43 mL, 5.00 eq) at 25 °C. The mixture was stirred at 25 °C for 12 h. LCMS showed 4-([1,1′-biphenyl]-3-yl)-5-chloro-N-(piperidin-4-yl)pyrimidin-2-amine (Compound B) was consumed completely and the desired mass was detected. Water (8 mL) was added to reaction, the aqueous phase was extracted with EtOAc (8 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 1:1) to give the title compound (008-2) (950 mg, 1.57 mmol, 95.6% yield) as a yellow oil. LCMS (ES+): m/z 604.3 [M+H]+.

Synthesis of 1-(4-((4-([1,1′-biphenyl]-3-y)-5-chloropyrimidin-2-yl)amino)piperidin-1-yl)-3-(piperidin-4-yl)propan-J-one (Compound 7). A solution of tert-butyl 4-(3-(4-((4-([1,1′-biphenyl]-3-yl)-5-chloropyrimidin-2-yl)amino)piperidin-1-yl)-3-oxopropyl)piperidine-1-carboxylate (008-2) (300 mg, 497 μmol, 1.00 eq) in TFA (1 mL) and DCM (5 mL) was stirred at 25 °C for 1 h. LCMS showed tert-butyl 4-(3-(4-((4-([1,1′-biphenyl]-3-yl)-5-chloropyrimidin-2-yl)amino)piperidin-1-yl)-3-oxopropyl)piperidine-1-carboxylate (008-2) was consumed completely and the desired mass was detected. The reaction mixture was concentrated under reduced pressure. The residue was purified by prep-HPLC (column: Phenomenex luna C18 80*40 mm*3 μm; mobile phase: [water (FA)-ACN]; B %: 25%-65%, 8 min) to give the title compound (Compound 7) (123 mg, 244 μmol, 49.1% yield, 99.8% purity) as a white solid. LCMS (ES+): m/z 504.2 [M+H]+.

### Example 9. Synthesis of 1-(4-((4-([1,1′-biphenyl]-3-yl)-5-chloropyrimidin-2-yl)amino)piperidin-1-yl)-3-(1-acetylpiperidin-4-yl)propan-1-one (Compound 8)

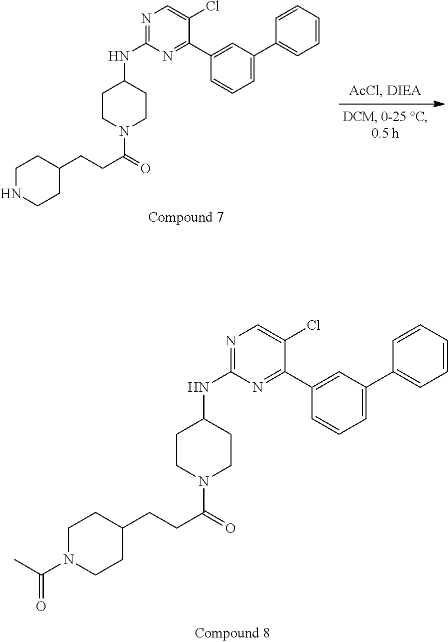 [SMILES: O=C(CCC1CCNCC1)N1CCC(Nc2ncc(Cl)c(-c3cccc(-c4ccccc4)c3)n2)CC1 ; CC(=O)N1CCC(CCC(=O)N2CCC(Nc3ncc(Cl)c(-c4cccc(-c5ccccc5)c4)n3)CC2)CC1]

Synthesis of 1-(4-((4-([1,1′-biphenyl]-3-yl)-5-chloropyrimidin-2-yl)amino)piperidin-1-yl)-3-(1-acetylpiperidin-4-yl)propan-J-one (Compound 8). To a solution of 1-(4-((4-([1,1′-biphenyl]-3-yl)-5-chloropyrimidin-2-yl)amino)piperidin-1-yl)-3-(piperidin-4-yl)propan-1-one (Compound 7) (200 mg, 397 μmol, 1.00 eq, TFA) in DCM (3 mL) were added DIEA (154 mg, 1.19 mmol, 207 μL, 3.00 eq) and AcCl (37.4 mg, 476 μmol, 34.0 μL, 1.20 eq) at 0 °C. The mixture was stirred at 25 °C for 0.5 h. LCMS showed 1-(4-((4-([1,1′-biphenyl]-3-yl)-5-chloropyrimidin-2-yl)amino)piperidin-1-yl)-3-(piperidin-4-yl)propan-1-one (Compound 7) was consumed completely and the desired mass was detected. The reaction mixture was concentrated under reduced pressure. The residue was purified by prep-HPLC (column: Waters Xbridge Prep OBD C18 150*40 mm*10 μm; mobile phase: [water(NH₄HCO₃)-ACN]; B %: 55%-85%, 8 min) to give the title compound (Compound 8) (28.4 mg, 51.2 μmol, 12.9% yield, 98.4% purity) as a pink solid. LCMS (ES+): m/z 546.2 [M+H]+.

### Example 10. Synthesis of 1-(4-((4-([1,1′-biphenyl]-3-yl)-5-chloropyrimidin-2-yl)amino)piperidin-1-yl)-2-(1-methylpiperidin-4-yl)ethan-1-one (Compound 9)

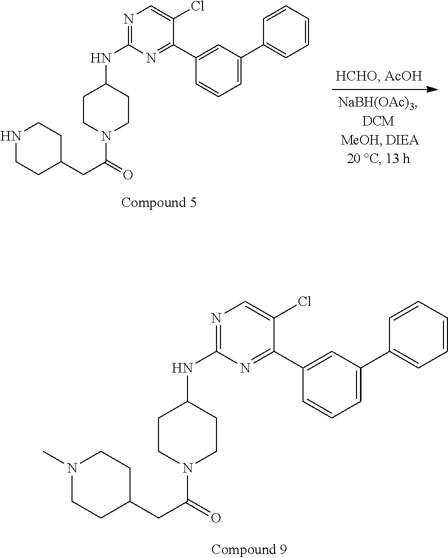 [SMILES: O=C(CC1CCNCC1)N1CCC(Nc2ncc(Cl)c(-c3cccc(-c4ccccc4)c3)n2)CC1 ; CN1CCC(CC(=O)N2CCC(Nc3ncc(Cl)c(-c4cccc(-c5ccccc5)c4)n3)CC2)CC1]

Synthesis of 1-(4-((4-([1,1′-biphenyl]-3-yl)-5-chloropyrimidin-2-yl)amino)piperidin-1-yl)-2-(1-methylpiperidin-4-yl)ethan-1-one (Compound 9). To a solution of 1-(4-((4-([1,1′-biphenyl]-3-yl)-5-chloropyrimidin-2-yl)amino)piperidin-1-yl)-2-(piperidin-4-yl)ethan-1-one (Compound 5) (300 mg, 612 μmol, 1.00 eq) and aq. HCHO (845 mg, 10.4 mmol, 775 μL, 37.0% purity, 17.0 eq) in DCM (4 mL) and MeOH (2 mL) was added DIEA (79.1 mg, 612 μmol, 107 μL, 1.00 eq) to adjust pH to 8 at 20 °C. The mixture was stirred at 20 °C for 0.5 h. To the reaction was added AcOH (3.68 mg, 61.2 μmol, 3.50 μL, 0.100 eq) to adjust pH to 5 at 20 °C. The mixture was stirred at 20 °C for 0.5 h. To the reaction mixture was added NaBH(OAc)₃ (156 mg, 735 μmol, 1.20 eq) at 20 °C for 12 h. LCMS showed that 1-(4-((4-([1,1′-biphenyl]-3-yl)-5-chloropyrimidin-2-yl)amino)piperidin-1-yl)-2-(piperidin-4-yl)ethan-1-one (Compound 5) was consumed completely and the desired mass was detected. The mixture was concentrated in vacuo. The residue was purified by prep-HPLC (column: Phenomenex Luna C18 75*30 mm*3 μm; mobile phase: [water (FA)-ACN]; B %: 25%-65%, 8 min) to give the title compound (Compound 9) (33.9 mg, 61.5 μmol, 10.0% yield, 99.7% purity, FA) as a yellow solid. LCMS (ES+): m/z 504.2 [M+H]+.

### Example 11. Synthesis of (4-((4-([1,1′-biphenyl]-3-yl)-5-chloropyrimidin-2-yl)amino)piperidin-1-yl)(3-azaspiro[5.5]undecan-9-yl)methanone (Compound 11)

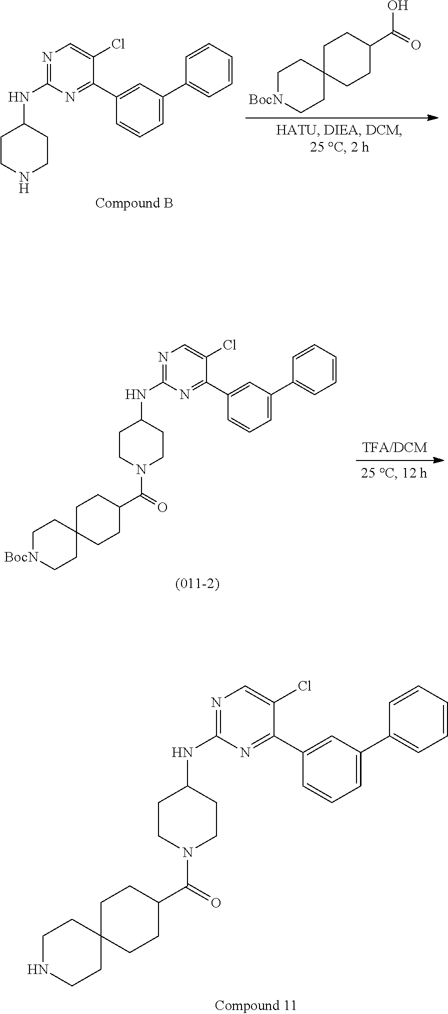 [SMILES: Clc1cnc(NC2CCNCC2)nc1-c1cccc(-c2ccccc2)c1 ; CC(C)(C)OC(=O)N1CCC2(CCC(C(=O)O)CC2)CC1 ; CC(C)(C)OC(=O)N1CCC2(CCC(C(=O)N3CCC(Nc4ncc(Cl)c(-c5cccc(-c6ccccc6)c5)n4)CC3)CC2)CC1 ; O=C(C1CCC2(CCNCC2)CC1)N1CCC(Nc2ncc(Cl)c(-c3cccc(-c4ccccc4)c3)n2)CC1]

Synthesis of tert-butyl 9-(4-((4-([1,1′-biphenyl]-3-yl)-5-chloropyrimidin-2-yl)amino)piperidine-1-carbonyl)-3-azaspiro[5.5]undecane-3-carboxylate (011-2). To a solution of 4-([1,1′-biphenyl]-3-yl)-5-chloro-N-(piperidin-4-yl)pyrimidin-2-amine (Compound B) (1.35 g, 2.82 mmol, 1.00 eq, TFA) in DCM (10 mL) were added HATU (1.61 g, 4.23 mmol, 1.50 eq), DIEA (1.82 g, 14.1 mmol, 2.46 mL, 5.00 eq) and 3-tert-butoxycarbonyl-3-azaspiro[5.5]undecane-9-carboxylic acid (1.01 g, 3.38 mmol, 1.20 eq) at 25 °C. The mixture was stirred at 25 °C for 2 h. LCMS showed 4-([1,1′-biphenyl]-3-yl)-5-chloro-N-(piperidin-4-yl)pyrimidin-2-amine (Compound B) was consumed completely and the desired mass was detected. Water (8 mL) was added to reaction, the aqueous phase was extracted with EtOAc (24 mL), the organic layer was dried over Na₂SO₄, filtered and the filtrate was concentrated under reduced pressure to give the title compound (011-2) (1.60 g, crude) as a white solid. LCMS (ES+): m/z 644.4 [M+H]⁺.

Synthesis of (4-((4-([1,1′-biphenyl]-3-yl)-5-chloropyrimidin-2-yl)amino)piperidin-1-yl)(3-azaspiro[5.5]undecan-9-yl)methanone (Compound 11). To a solution of tert-butyl 9-(4-((4-([1,1′-biphenyl]-3-yl)-5-chloropyrimidin-2-yl)amino)piperidine-1-carbonyl)-3-azaspiro[5.5]undecane-3-carboxylate (011-2) (1.60 g, 2.48 mmol, 1.00 eq) in DCM (9 mL) was added TFA (6.72 g, 58.9 mmol, 4.36 mL, 23.7 eq) at 25 °C. The mixture was stirred at 25 °C for 12 h. LCMS showed tert-butyl 9-(4-((4-([1,1′-biphenyl]-3-yl)-5-chloropyrimidin-2-yl)amino)piperidine-1-carbonyl)-3-azaspiro[5.5]undecane-3-carboxylate (011-2) was consumed completely and the desired mass was detected. The reaction mixture was concentrated under reduced pressure to remove solvent. The residue was purified by acid prep-HPLC (column: Phenomenex C18 80*40 mm*3 μm; mobile phase: [water (NH₄HCO₃)-ACN]; B %: 30%-60%, 8 min) to give the title compound (Compound 11) (900 mg, 1.34 mmol, 53.8% yield, 97.7% purity, TFA) as a white solid. LCMS (ES+): m/z 544.4 [M+H]+.

### Example 12. Synthesis of (4-((4-([1,1′-biphenyl]-3-yl)-5-chloropyrimidin-2-yl)amino)piperidin-1-yl)(3-methyl-3-azaspiro[5.5]undecan-9-yl)methanone (Compound 12)

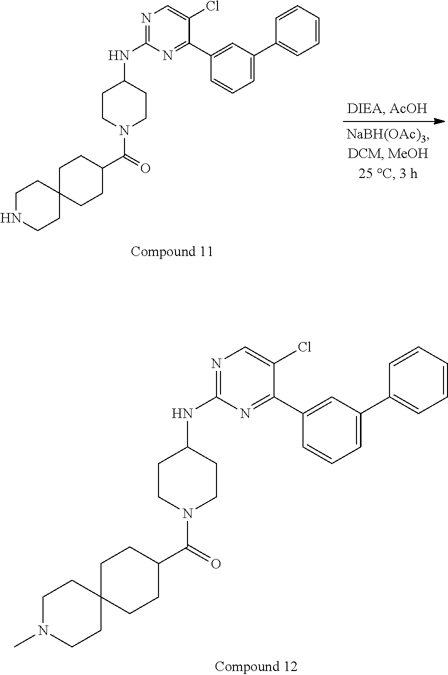 [SMILES: O=C(C1CCC2(CCNCC2)CC1)N1CCC(Nc2ncc(Cl)c(-c3cccc(-c4ccccc4)c3)n2)CC1 ; CN1CCC2(CCC(C(=O)N3CCC(Nc4ncc(Cl)c(-c5cccc(-c6ccccc6)c5)n4)CC3)CC2)CC1]

Synthesis of (4-((4-([1,1′-biphenyl]-3-yl)-5-chloropyrimidin-2-yl)amino)piperidin-1-yl)(3-methyl-3-azaspiro[5.5]undecan-9-yl)methanone (Compound 12). To a solution of (4-((4-([1,1′-biphenyl]-3-yl)-5-chloropyrimidin-2-yl)amino)piperidin-1-yl)(3-azaspiro[5.5]undecan-9-yl)methanone (Compound 11) (230 mg, 349 μmol, 1.00 eq, TFA) and aq. formaldehyde (482 mg, 5.94 mmol, 270 μL, 37.0% purity, 17.0 eq) in DCM (4 mL) and MeOH (2 mL) was added DIEA (45.2 mg, 349 μmol, 60.9 μL, 1.00 eq) at 25 °C. DIEA was added dropwise until the pH=8. The mixture was stirred at 25 °C for 0.5 h. AcOH (21.0 mg, 349 μmol, 20.0 μL, 1.00 eq) was added dropwise until pH=6 at 25 °C. The mixture was stirred at 25 °C for 0.5 h. NaBH(OAc)₃ (88.9 mg, 419 μmol, 1.20 eq) was added to the mixture at 25 °C. The mixture was stirred at 25 °C for 2 h. LCMS showed (4-((4-([1,1′-biphenyl]-3-yl)-5-chloropyrimidin-2-yl)amino)piperidin-1-yl)(3-azaspiro[5.5]undecan-9-yl)methanone (Compound 11) was consumed completely and the desired mass was detected. The reaction mixture was concentrated under reduced pressure to remove solvent. The residue was purified by prep-HPLC (column: Phenomenex Luna C18 200*40 mm*10 μm; mobile phase: [water (FA)-ACN]; B %: 10%-50%, 8 min) to give the title compound (Compound 12) (33.6 mg, 55.6 μmol, 15.9% yield, 100% purity, FA) as a white solid. LCMS (ES+): m/z 558.3 [M+H]+.

### Example 13. Synthesis of 1-(9-(4-((4-([1,1′-biphenyl]-3-yl)-5-chloropyrimidin-2-yl)amino)piperidine-1-carbonyl)-3-azaspiro[5.5]undecan-3-yl)ethan-1-one (Compound 13)

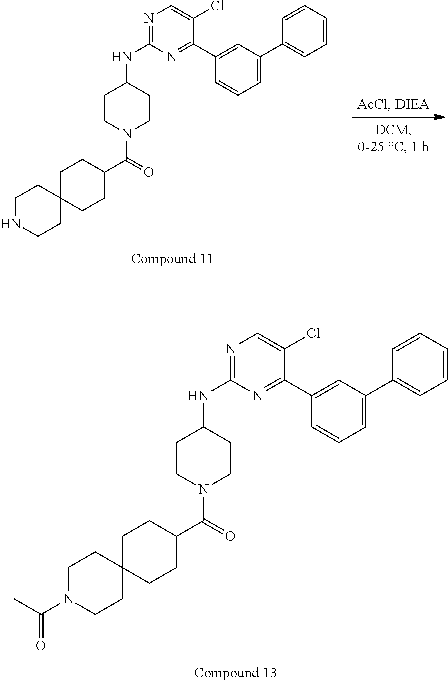 [SMILES: O=C(C1CCC2(CCNCC2)CC1)N1CCC(Nc2ncc(Cl)c(-c3cccc(-c4ccccc4)c3)n2)CC1 ; CC(=O)N1CCC2(CCC(C(=O)N3CCC(Nc4ncc(Cl)c(-c5cccc(-c6ccccc6)c5)n4)CC3)CC2)CC1]

Synthesis of 1-(9-(4-((4-([1,1′-biphenyl]-3-yl)-5-chloropyrimidin-2-yl)amino)piperidine-1-carbonyl)-3-azaspiro[5.5]undecan-3-yl)ethan-1-one (Compound 13). To a solution of (4-((4-([1,1′-biphenyl]-3-yl)-5-chloropyrimidin-2-yl)amino)piperidin-1-yl)(3-azaspiro[5.5]undecan-9-yl)methanone (Compound 11) (230 mg, 349 μmol, 1.00 eq, TFA) in DCM (6 mL) were added DIEA (135 mg, 1.05 mmol, 183 μL, 3.00 eq) and acetyl chloride (32.9 mg, 419 μmol, 29.9 μL, 1.20 eq) at 0 °C. The mixture was stirred at 25 °C for 1 h. LCMS showed (4-((4-([1,1′-biphenyl]-3-yl)-5-chloropyrimidin-2-yl)amino)piperidin-1-yl)(3-azaspiro[5.5]undecan-9-yl)methanone (Compound 11) was consumed completely and the desired mass was detected. The reaction mixture was concentrated under reduced pressure to remove solvent. The residue was purified by prep-HPLC (column: Phenomenex Luna C18 200*40 mm*10 μm; mobile phase: [water(FA)-ACN]; B %: 45%-75%, 8 min) to give the title compound (Compound 13) (36.2 mg, 57.3 μmol, 16.4% yield, 100% purity, FA) as a white solid. LCMS (ES+): m/z 586.3 [M+H]+.

### Example 14. Synthesis of 5-((4-([1,1′-biphenyl]-3-yl)-5-chloropyrimidin-2-yl)amino)-1-methylpiperidin-2-one (Compound 14)

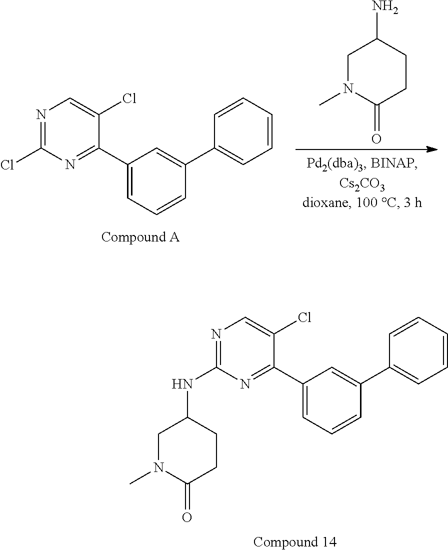

Synthesis of 5-((4-([1,1′-biphenyl]-3-yl)-5-chloropyrimidin-2-yl)amino)-1-methylpiperidin-2-one (Compound 14). To a solution of 5-amino-1-methylpiperidin-2-one (200 mg, 664 μmol, 1.00 eq) and 4-([1,1′-biphenyl]-3-yl)-2,5-dichloropyrimidine (Compound A) (85.1 mg, 664 μmol, 1.00 eq) in dioxane (3 mL) were added BINAP (24.8 mg, 39.8 μmol, 0.0600 eq), Cs₂CO₃ (432 mg, 1.33 mmol, 2.00 eq) and Pd₂(dba)₃ (24.3 mg, 26.6 μmol, 0.0400 eq) at 25 °C. The mixture was stirred at 100 °C for 3 h under N₂ atmosphere. LCMS showed 5-amino-1-methylpiperidin-2-one consumed and the desired mass was detected. The mixture was diluted with H₂O (10 mL) and then extracted with EtOAc (5 mL*3). The combined organic layers were dried over Na₂SO₄, filtered and the filtrate was concentrated in vacuo. The mixture was purified by prep-HPLC (column: Phenomenex luna C18 80*40 mm*3 μm; mobile phase: [water(FA)-ACN]; B %: 45%-85%, 8 min) to give the title compound (Compound 14) (36.5 mg, 90.9 μmol, 13.7% yield, 97.8% purity) as a white solid. LCMS (ES+): m/z 393.0 [M+H]+.

### Example 15. Synthesis of 4-((4-([1,1′-biphenyl]-3-yl)-5-chloropyrimidin-2-yl)amino)piperidin-2-one (Compound 15)

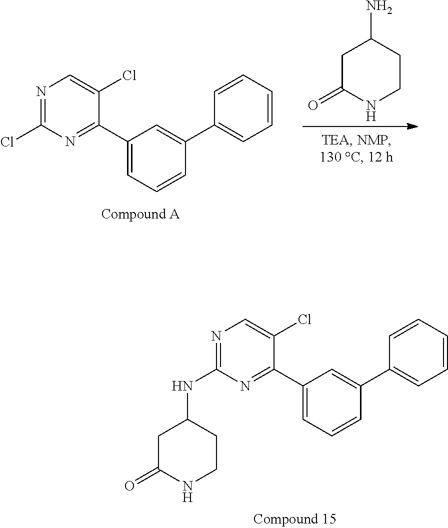

Synthesis of 4-((4-([1, 1′-biphenyl]-3-yl)-5-chloropyrimidin-2-yl)amino)piperidin-2-one (Compound 15). To a stirred solution of 4-aminopiperidin-2-one (50.0 mg, 332 μmol, 1.00 eq) and 2, 5-dichloro-4-(3-phenylphenyl)pyrimidine (Compound A) (110 mg, 365 μmol, 1.10 eq) in NMP (2 mL) was added TEA (101 mg, 996 μmol, 139 μL, 3.00 eq). The mixture was stirred at 130 °C for 12 h. LCMS showed the desired compound was detected. The reaction mixture was concentrated. The residue was purified by prep-HPLC (column: Phenomenex luna C18 80*40 mm*3 μm; mobile phase: [water (FA)-ACN]; B %: 30%-70%, 8 min) to give the title compound (Compound 15) (17.7 mg, 46.7 μmol, 14.1% yield, 100.00% purity) as a white solid. LCMS (ES+): m/z 379.1 [M+H]+.

### Example 16. Synthesis of 4-((4-([1,1′-biphenyl]-3-yl)-5-chloropyrimidin-2-yl)amino)-1-methylpiperidin-2-one (Compound 16)

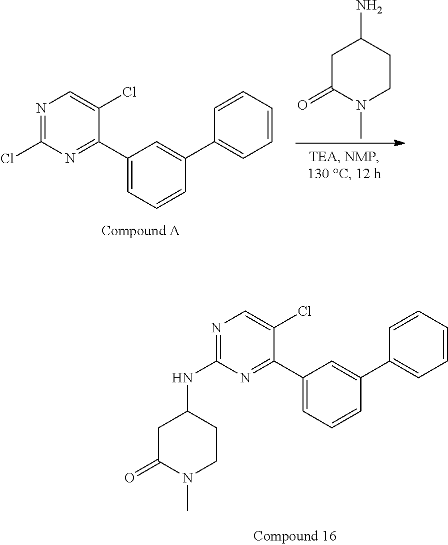

Synthesis of 4-((4-([1,1′-biphenyl]-3-yl)-5-chloropyrimidin-2-yl)amino)-1-methylpiperidin-2-one (Compound 16). To a stirred solution of 4-amino-1-methylpiperidin-2-one (50.0 mg, 390 μmol, 1.10 eq) and 2,5-dichloro-4-(3-phenylphenyl)pyrimidine (Compound A) (107 mg, 355 μmol, 1.00 eq) in NMP (2 mL) was added TEA (108 mg, 1.06 mmol, 148 μL, 3.00 eq). The mixture was stirred at 130 °C for 12 h. LCMS showed the desired MS was detected. The reaction mixture was concentrated. The residue was purified by prep-HPLC (column: Phenomenex luna C18 80*40 mm*3 μm; mobile phase: [water (FA)-ACN]; B %: 30%-70%, 8 min) to give the title compound (Compound 16) (26.3 mg, 66.7 μmol, 18.8% yield, 99.6% purity) as a white solid. LCMS (ES+): m/z 393.1 [M+H]+.

### Example 17. Synthesis of 5-((4-([1,1′-biphenyl]-3-yl)-5-chloropyrimidin-2-yl)amino)piperidin-2-one (Compound 17)

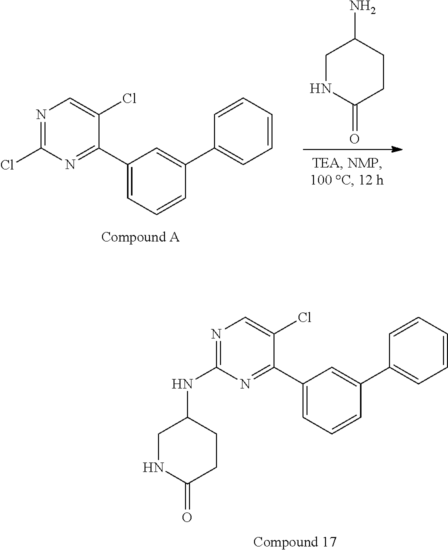 [SMILES: Clc1ncc(Cl)c(-c2cccc(-c3ccccc3)c2)n1 ; NC1CCC(=O)NC1 ; O=C1CCC(Nc2ncc(Cl)c(-c3cccc(-c4ccccc4)c3)n2)CN1]

Synthesis of 5-((4-([1,1′-biphenyl]-3-yl)-5-chloropyrimidin-2-yl)amino)piperidin-2-one (Compound 17). To a solution of 5-aminopiperidin-2-one (100 mg, 664 μmol, 1.10 eq), 2,5-dichloro-4-(3-phenylphenyl)pyrimidine (Compound A) (182 mg, 604 μmol, 1.00 eq) and TEA (183 mg, 1.81 mmol, 252 μL, 3.00 eq) in NMP (2 mL) was degassed and purged with N₂ for 3 times, and then the mixture was stirred at 100 °C for 12 h under N₂ atmosphere. LCMS showed 5-aminopiperidin-2-one was consumed completely and desired mass was detected. The reaction mixture was concentrated under reduced pressure to get a residue. The residue was purified by prep-HPLC (column: Phenomenex luna C18 80*40 mm*3 μm; mobile phase: [water (FA)-ACN]; B %: 30%-70%, 8 min) to give the title compound (Compound 17) (67.7 mg, 179 μmol, 29.6% yield, 100.00% purity) as a white solid. LCMS (ES+): m/z 379.1 [M+H]+.

### Example 18. Synthesis of (S)-1-(3-((4-([1,1′-biphenyl]-3-yl)-5-chloropyrimidin-2-yl)amino)piperidin-1-yl)ethan-1-one (Compound 18)

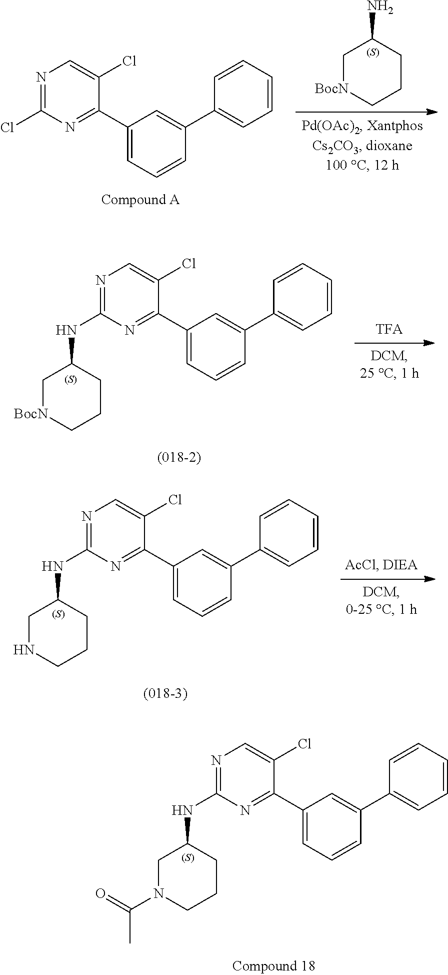

Synthesis of tert-butyl (S)-3-((4-([1,1′-biphenyl]-3-yl)-5-chloropyrimidin-2-yl)amino)piperidine-1-carboxylate (018-2). To a solution of 4-([1,1′-biphenyl]-3-yl)-2,5-dichloropyrimidine (Compound A) (300 mg, 996 μmol, 1.00 eq) and tert-butyl (3S)-3-aminopiperidine-1-carboxylate (200 mg, 996 μmol, 1.00 eq) in dioxane (5 mL) were added Xantphos (17.3 mg, 29.9 μmol, 0.0300 eq), Cs₂CO₃ (649 mg, 1.99 mmol, 2.00 eq) and Pd(OAc)₂ (11.2 mg, 49.8 μmol, 0.0500 eq) at 25 °C under N₂ atmosphere. The mixture was stirred at 100 °C for 12 h under N₂. LCMS showed that the desired mass was detected. Water (5 mL) was added and the mixture was extracted with EtOAc (12 mL*3). The organic layers were dried over Na₂SO₄, filtered and the filtrate was concentrated under reduced pressure to give a residue. The residue was purified by prep-TLC (SiO₂, Petroleum ether:Ethyl acetate=3:1) to give the title compound (018-2) (120 mg, 258 μmol, 25.9% yield) as a white solid. LCMS (ES+): m/z 465.2 [M+H]+.

Synthesis of (S)-4-([1,1′-biphenyl]-3-yl)-5-chloro-N-(piperidin-3-yl)pyrimidin-2-amine (018-3). To a solution of tert-butyl (S)-3-((4-([1,1′-biphenyl]-3-yl)-5-chloropyrimidin-2-yl)amino)piperidine-1-carboxylate (018-2) (120 mg, 258 μmol, 1.00 eq) in DCM (3 mL) was added TFA (1.54 g, 13.5 mmol, 1.00 mL, 52.3 eq) at 25 °C. The mixture was stirred at 25 °C for 1 h. LCMS showed the desired mass was detected. The reaction mixture was concentrated under reduced pressure to remove solvent to give the title compound (018-3) (120 mg, crude, TFA) as a yellow oil. LCMS (ES+): m/z 365.0 [M+H]+.

Synthesis of (S)-1-(3-((4-([1,1′-biphenyl]-3-yl)-5-chloropyrimidin-2-yl)amino)piperidin-1-yl)ethan-1-one (Compound 18). To a solution of (S)-4-([1,1′-biphenyl]-3-yl)-5-chloro-N-(piperidin-3-yl)pyrimidin-2-amine (018-3) (110 mg, 230 μmol, 1.00 eq, TFA) in DCM (3 mL) were added DIEA (89.1 mg, 689 μmol, 120 μL, 3.00 eq) and acetyl chloride (21.6 mg, 276 μmol, 19.7 μL, 1.20 eq) at 0 °C. The mixture was stirred at 25 °C for 1 h. LCMS showed (S)-4-([1,1′-biphenyl]-3-yl)-5-chloro-N-(piperidin-3-yl)pyrimidin-2-amine (018-3) was consumed completely and the desired mass was detected. Water (3 mL) was added to the mixture at 25 °C. Then the reaction mixture was extracted with EtOAc (3 mL*3). The organic layers were dried over Na₂SO₄, filtered and the filtrate was concentrated under reduced pressure to give a residue. The residue was purified by prep-HPLC (column: Phenomenex Luna C18 75*30 mm*3 μm; mobile phase: [water (FA)-ACN]; B %: 45%-85%, 8 min) to give the title compound (Compound 18) (43.3 mg, 104 μmol, 45.3% yield, 97.7% purity) as a yellow solid. LCMS (ES+): m/z 407.0 [M+H]+.

### Example 19. Synthesis of trans-4-((4-([1,1′-biphenyl]-3-yl)-5-chloropyrimidin-2-yl)amino)cyclohexane-1-carboxamide (Compound 19)

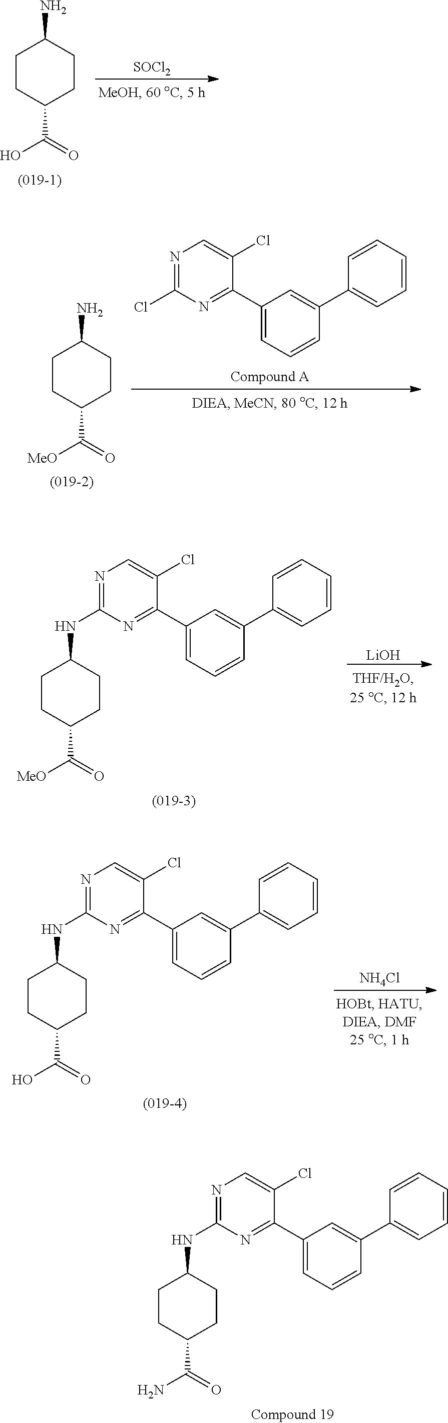

Synthesis of methyl trans-4-aminocyclohexane-1-carboxylate (019-2). To a solution of trans-4-aminocyclohexane-1-carboxylic acid (019-1) (5.00 g, 34.9 mmol, 1.00 eq) in MeOH (120 mL) was added SOCl₂ (8.31 g, 69.8 mmol, 5.07 mL, 2.00 eq) at 0 °C under N₂ atmosphere. The mixture was stirred at 60 °C for 5 h. LCMS showed the desired mass was detected. The reaction mixture was concentrated under reduced pressure to remove solvent to give the title compound (019-2) (5.49 g, crude) as a white solid. LCMS (ES+): m/z 158.1 [M+H]+.

Synthesis of methyl trans-4-((4-([1,1′-biphenyl]-3-yl)-5-chloropyrimidin-2-yl)amino)cyclohexane-1-carboxylate (019-3). To a solution of methyl trans-4-aminocyclohexane-1-carboxylate (019-2) (5.40 g, 34.4 mmol, 2.50 eq) in MeCN (100 mL) were added DIEA (1.78 g, 13.7 mmol, 2.39 mL, 1.00 eq) and 2,5-dichloro-4-(3-phenylphenyl)pyrimidine (Compound A) (4.14 g, 13.7 mmol, 1.00 eq) at 25 °C. The mixture was stirred at 80 °C for 12 h. LCMS showed ~70.5% of 2,5-dichloro-4-(3-phenylphenyl)pyrimidine (Compound A) was remaining and desired mass was detected. The reaction mixture was concentrated under reduced pressure to remove solvent. The residue was purified by column chromatography (SiO₂, Petroleum ether:Ethyl acetate=1:0 to 3:1) to give the title compound (019-3) (1.04 g, 2.46 mmol, 17.9% yield) as a yellow oil. LCMS (ES+): m/z 422.2 [M+H]+.

Synthesis of trans-4-((4-([1,1′-biphenyl]-3-yl)-5-chloropyrimidin-2-yl)amino)cyclohexane-1-carboxylic acid (019-4). To a solution of methyl trans-4-((4-([1,1′-biphenyl]-3-yl)-5-chloropyrimidin-2-yl)amino)cyclohexane-1-carboxylate (019-3) (500 mg, 1.19 mmol, 1.00 eq) in THE (12 mL) and H₂O (4 mL) was added LiOH·H₂O (249 mg, 5.93 mmol, 5.00 eq) at 25 °C. The mixture was stirred at 25 °C for 12 h. LCMS showed methyl trans-4-((4-([1,1′-biphenyl]-3-yl)-5-chloropyrimidin-2-yl)amino)cyclohexane-1-carboxylate (019-3) was consumed completely and the desired mass was detected. Aq. HCl (10.0%, 4.00 mL) was added dropwise until the pH=3. The aqueous solution was extracted with EtOAc (5 mL*3). The organic layers were combined, dried over anhydrous Na₂SO₄, filtered and concentrated under reduced pressure to give the title compound (019-4) (210 mg, crude, HCl) as a yellow solid. LCMS (ES+): m/z 408.1 [M+H]+.

Synthesis of trans-4-((4-([1,1′-biphenyl]-3-yl)-5-chloropyrimidin-2-yl)amino)cyclohexane-1-carboxamide (Compound 19). To a solution of trans-4-((4-([1,1′-biphenyl]-3-yl)-5-chloropyrimidin-2-yl)amino)cyclohexane-1-carboxylic acid (019-4) (180 mg, 405 μmol, 1.00 eq, HCl) in DMF (2 mL) were added NH₄Cl (32.5 mg, 608 μmol, 1.50 eq), HATU (185 mg, 486 μmol, 1.20 eq), DIEA (262 mg, 2.03 mmol, 353 μL, 5.00 eq) and HOBt (27.4 mg, 203 μmol, 0.500 eq) at 25 °C. The mixture was stirred at 25 °C for 1 h. LCMS showed the desired mass was detected. Water (2 mL) was added to the mixture at 25 °C. Then the reaction mixture was extracted with EtOAc (1 mL*3). The organic layers were dried over anhydrous sodium sulfate, filtered and the filtrate was concentrated under reduced pressure to give a residue. The residue was purified by prep-HPLC (column: Phenomenex Luna C18 75*30 mm*3 μm; mobile phase: [water (FA)-ACN]; B %: 35%-75%, 8 min) to give the title compound (Compound 19) (54.1 mg, 131 μmol, 32.3% yield, 98.4% purity) as a white solid. LCMS (ES+): m/z 407.0 [M+H]+.

### Example 20. Synthesis of 1-((3S)-3-((5-chloro-4-(3-phenylpyrrolidin-1-yl)pyrimidin-2-yl)amino)piperidin-1-yl)ethan-1-one (Compound 20)

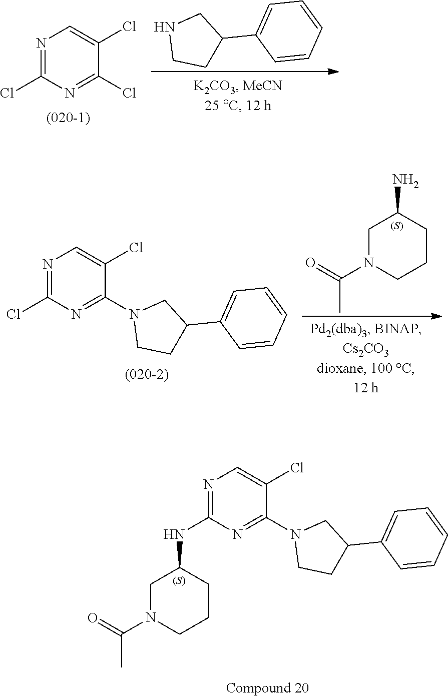

Synthesis of 2,5-dichloro-4-(3phenylpyrrolidin-1yl)pyrimidine (020-2). To a solution of 2,4,5-trichloropyrimidine (020-1) (1.02 g, 5.56 mmol, 1.00 eq) and 3-phenylpyrrolidine (900 mg, 6.11 mmol, 1.10 eq) in MeCN (20 mL) was added K₂CO₃ (2.30 g, 16.7 mmol, 3.00 eq) at 25 °C. The mixture was stirred at 25 °C for 12 h. LCMS showed desired mass was detected. The reaction mixture was diluted with H₂O (10 mL) and extracted with EtOAc (10 mL*3). The combined organic layers were dried over Na₂SO₄, filtered and concentrated under reduced pressure. The residue was purified by MPLC (SiO₂, PE:EtOAc=5:1) to give the title compound (020-2) (1.40 g, 4.28 mmol, 77.1% yield, 90.0% purity) as a white solid. LCMS (ESI+): m/z 294.2 [M+H]+.

Synthesis of 1-((3S)-3-((5-chloro-4-(3-phenylpyrrolidin-1-yl)pyrimidin-2-yl)amino)piperidin-1-yl)ethan-1-one (Compound 20). To a solution of 2,5-dichloro-4-(3phenylpyrrolidin-1-yl)pyrimidine (020-2) (100 mg, 340 μmol, 1.00 eq) and 1-[(3S)-3-amino-1-piperidyl]ethanone (67.7 mg, 476 μmol, 1.40 eq) in dioxane (3 mL) were added Cs₂CO₃ (277 mg, 850 μmol, 2.50 eq) and BINAP (21.2 mg, 34.0 μmol, 0.100 eq) at 25 °C, then Pd₂(dba)₃ (31.1 mg, 34.0 μmol, 0.100 eq) was added under N₂. The mixture was stirred at 100 °C for 12 h under N₂. LCMS showed desired mass was detected. The reaction mixture was diluted with H₂O (10 mL) and extracted with EtOAc (10 mL*3). The combined organic layers were dried over Na₂SO₄, filtered and concentrated under reduced pressure. The residue was purified by prep-HPLC (column: Phenomenex C18 80*40 mm*3 μm; mobile phase: [water (NH₄HCO₃)-ACN]; B %: 30%-60%, 8 min) to give the title compound (Compound 20) (73.5 mg, 183 μmol, 26.9% yield, 99.5% purity) as a white solid. LCMS (ESI+): m/z 400.1 [M+H]+.

### Example 21. Synthesis of 1-(3-((4-([1,1′-biphenyl]-3-yl)-5-chloropyrimidin-2-yl)amino)piperidin-1-yl)ethan-1-one (Compound 21)

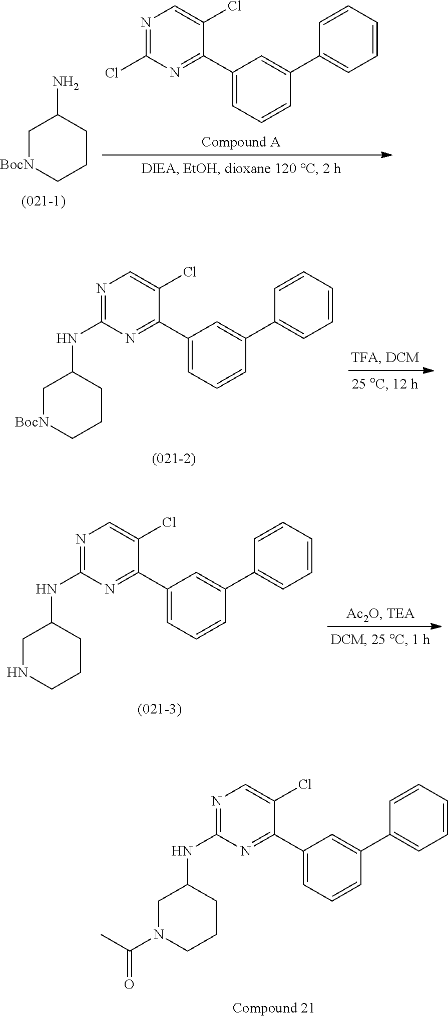 [SMILES: CC(C)(C)OC(=O)N1CCCC(N)C1 ; Clc1ncc(Cl)c(-c2cccc(-c3ccccc3)c2)n1 ; CC(C)(C)OC(=O)N1CCCC(Nc2ncc(Cl)c(-c3cccc(-c4ccccc4)c3)n2)C1 ; Clc1cnc(NC2CCCNC2)nc1-c1cccc(-c2ccccc2)c1 ; CC(=O)N1CCCC(Nc2ncc(Cl)c(-c3cccc(-c4ccccc4)c3)n2)C1]

Synthesis of tert-butyl 3-((4-([1,1′-biphenyl]-3-yl)-5-chloropyrimidin-2-yl)amino)piperidine-1-carboxylate (021-2). A mixture of tert-butyl 3-aminopiperidine-1-carboxylate (021-1) (366 mg, 1.83 mmol, 1.10 eq), 2,5-dichloro-4-(3-phenylphenyl)pyrimidine (Compound A) (500 mg, 1.66 mmol, 1.00 eq), DIEA (644 mg, 4.98 mmol, 868 μL, 3.00 eq) in EtOH (5 mL) and dioxane (5 mL) was stirred at 120 °C for 2 h. LCMS showed tert-butyl 3-aminopiperidine-1-carboxylate (021-1) was consumed completely and desired mass was detected. The reaction mixture was concentrated under reduced pressure to get a residue, and the residue was purified by flash silica gel chromatography (ISCO®; 10 g SepaFlash® Silica Flash Column, Eluent of 0~30% Ethyl acetate/Petroleum ether gradient @ 80 mL/min) to give the title compound (021-2) (520 mg, 1.12 mmol, 67.4% yield) as a yellow solid. LCMS (ES−): m/z 463.2 [M−H]−.

Synthesis of 4-([1,1′-biphenyl]-3-yl)-5-chloro-N-(piperidin-3-yl)pyrimidin-2-amine (021-3). To a solution of tert-butyl 3-((4-([1,1′-biphenyl]-3-yl)-5-chloropyrimidin-2-yl)amino)piperidine-1-carboxylate (021-2) (520 mg, 1.12 mmol, 1.00 eq) in DCM (5 mL) was added TFA (1 mL). The mixture was stirred at 25 °C for 12 h. LCMS showed tert-butyl 3-((4-([1,1′-biphenyl]-3-yl)-5-chloropyrimidin-2-yl)amino)piperidine-1-carboxylate (021-2) was consumed completely and desired mass was detected. The reaction mixture was concentrated. The residue was purified by prep-HPLC (column: Phenomenex Luna C18 75*30 mm*3 μm; mobile phase: [water (FA)-ACN]; B %: 20%-60%, 8 min) to give the title compound (021-3) (230 mg, crude) as a white solid. LCMS (ES+): m/z 365.1 [M+H]+.

Synthesis of 1-(3-((4-([1,1′-biphenyl]-3-yl)-5-chloropyrimidin-2-yl)amino)piperidin-1-yl)ethan-1-one (Compound 21). To a solution of 4-([1,1′-biphenyl]-3-yl)-5-chloro-N-(piperidin-3-yl)pyrimidin-2-amine (021-3) (130 mg, 356 μmol, 1.00 eq) and TEA (108 mg, 1.07 mmol, 149 μL, 3.00 eq) in DCM (3 mL) was added Ac₂O (54.6 mg, 534 μmol, 50.1 μL, 1.50 eq). The mixture was stirred at 25 °C for 1 h. LCMS showed 4-([1,1′-biphenyl]-3-yl)-5-chloro-N-(piperidin-3-yl)pyrimidin-2-amine (021-3) was consumed completely and desired mass was detected. The reaction mixture was concentrated. The residue was purified by prep-HPLC (column: Waters Xbridge BEH C18 100*30 mm*10 μm; mobile phase: [water (NH₄HCO₃)-ACN]; B %: 40%-80%, 8 min) to give the title compound (Compound 21) (41.0 mg, 99.7 μmol, 28.0% yield, 99.0% purity) as a white solid. LCMS (ES−): m/z 405.2 [M−H]−

### Example 22. Synthesis of 3-((4-([1,1′-biphenyl]-3-yl)-5-chloropyrimidin-2-yl)amino)cyclohexane-1-carboxamide (Compound 22)

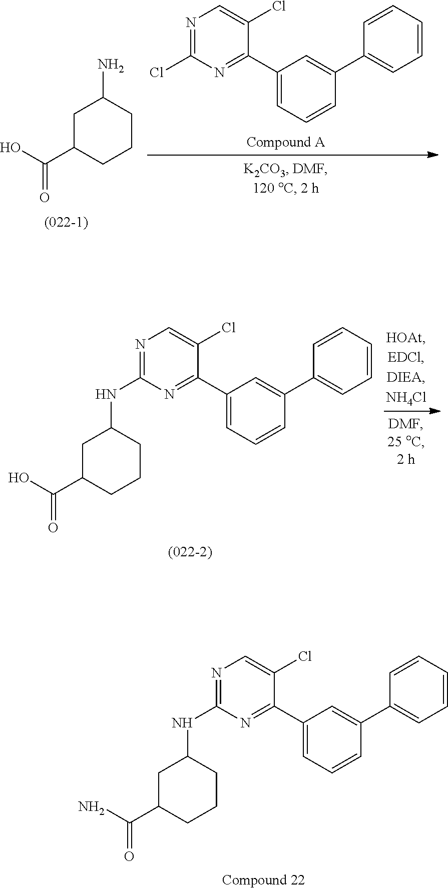 [SMILES: NC1CCCC(C(=O)O)C1 ; Clc1ncc(Cl)c(-c2cccc(-c3ccccc3)c2)n1 ; O=C(O)C1CCCC(Nc2ncc(Cl)c(-c3cccc(-c4ccccc4)c3)n2)C1 ; NC(=O)C1CCCC(Nc2ncc(Cl)c(-c3cccc(-c4ccccc4)c3)n2)C1]

Synthesis of 3-((4-([1,1′-biphenyl]-3-yl)-5-chloropyrimidin-2-yl)amino)cyclohexane-1-carboxylic acid (022-2). The mixture of 3-aminocyclohexane-1-carboxylic acid (022-1) (105 mg, 730 μmol, 1.10 eq), 2,5-dichloro-4-(3-phenylphenyl)pyrimidine (Compound A) (200 mg, 664 μmol, 1.00 eq), K₂CO₃ (22.0 mg, 1.66 mmol, 347 μL, 2.50 eq) in DMF (3 mL) was stirred at 120 °C for 2 h. LCMS showed the desired compound was detected. The reaction mixture was concentrated. The residue was purified by prep-HPLC (column: Waters Xbridge BEH C18 100*30 mm*10 μm; mobile phase: [water (NH₄HCO₃)-ACN]; B %: 25%-55%, 7 min) to give the title compound (022-2) (66.0 mg, 162 μmol, 24.4% yield) as a yellow oil. LCMS (ES+): m/z 408.1 [M+H]+.

Synthesis of 3-((4-([1,1′-biphenyl]-3-yl)-5-chloropyrimidin-2-yl)amino)cyclohexane-1-carboxamide (Compound 22). To a stirred solution of 3-((4-([1,1-biphenyl]-3-yl)-5-chloropyrimidin-2-yl)amino)cyclohexane-1-carboxylic acid (022-2) (66.0 mg, 162 μmol, 1.00 eq) in DMF (2 mL) were added HOAt (26.4 mg, 194 μmol, 27.2 μL, 1.20 eq), EDCI (37.2 mg, 194 μmol, 1.20 eq), DIEA (62.7 mg, 485 μmol, 84.6 μL, 3.00 eq) and NH₄Cl (17.3 mg, 324 μmol, 2.00 eq), the mixture was stirred at 25 °C for 2 h. LCMS showed the desired compound was detected. The reaction mixture was concentrated. The residue was purified by prep-HPLC (column: Phenomenex Luna 80*30 mm*3 μm; mobile phase: [water (FA)-ACN]; B %: 45%-70%, 8 min) to give the title compound (Compound 22) (11.3 mg, 27.8 μmol, 17.2% yield, 100.00% purity) as a white solid. LCMS (ES+): m/z 407.1 [M+H]⁺.

### Example 23. Synthesis of 1-(4-((4-([1,1′-biphenyl]-3-yl)-5-chloropyrimidin-2-yl)amino)piperidin-1-yl)ethan-1-one (Compound 23)

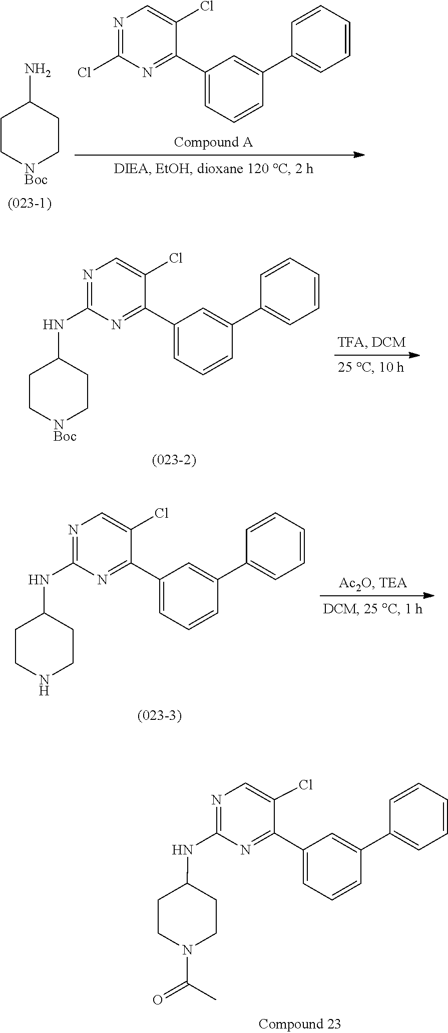 [SMILES: CC(C)(C)OC(=O)N1CCC(N)CC1 ; Clc1ncc(Cl)c(-c2cccc(-c3ccccc3)c2)n1 ; CC(C)(C)OC(=O)N1CCC(Nc2ncc(Cl)c(-c3cccc(-c4ccccc4)c3)n2)CC1 ; Clc1cnc(NC2CCNCC2)nc1-c1cccc(-c2ccccc2)c1 ; CC(=O)N1CCC(Nc2ncc(Cl)c(-c3cccc(-c4ccccc4)c3)n2)CC1]

Synthesis of tert-butyl 4-((4-([1,1′-biphenyl]-3-yl)-5-chloropyrimidin-2-yl)amino)piperidine-1-carboxylate (023-2). The solution of tert-butyl 4-aminopiperidine-1-carboxylate (023-1) (366 mg, 1.83 mmol, 1.10 eq), 2,5-dichloro-4-(3-phenylphenyl)pyrimidine (Compound A) (500 mg, 1.66 mmol, 1.00 eq) and DIEA (644 mg, 4.98 mmol, 868 μL, 3.00 eq) in EtOH (5 mL) and dioxane (5 mL) was stirred at 120 °C for 2 h. LCMS showed that tert-butyl 4-aminopiperidine-1-carboxylate (023-1) was consumed completely and the desired mass was detected. The reaction mixture was concentrated. The residue was purified by column chromatography (SiO₂, Petroleum ether:Ethyl acetate=10:1 to 1:1) to give the title compound (023-2) (390 mg, 839 μmol, 50.5% yield) as a yellow solid. LCMS (ES−): m/z 463.2 [M−H]−.

Synthesis of 4-([1,1′-biphenyl]-3-yl)-5-chloro-N-(piperidin-4-yl)pyrimidin-2-amine (Compound B) (023-3). To a stirred solution of tert-butyl 4-((4-([1,1′-biphenyl]-3-yl)-5-chloropyrimidin-2-yl)amino)piperidine-1-carboxylate (023-2) (389 mg, 837 μmol, 1.00 eq) in DCM (5 mL) was added TFA (95.4 mg, 837 μmol, 62.0 μL, 1.00 eq). The mixture was stirred at 25 °C for 10 h. LC-MS showed the desired compound was detected. The reaction mixture was concentrated to give the title compound (023-3) (200 mg, 548 μmol, 65.5% yield) as a yellow solid. LCMS (ES+): m/z 365.0 [M+H]+.

Synthesis of 1-(4-((4-([1,1′-biphenyl]-3-yl)-5-chloropyrimidin-2-yl)amino)piperidin-1-yl)ethan-1-one (Compound 23). To a solution of 4-([1,1′-biphenyl]-3-yl)-5-chloro-N-(piperidin-4-yl)pyrimidin-2-amine (Compound B) (023-3) (100 mg, 274 μmol, 1.00 eq) and TEA (83.2 mg, 822 μmol, 114 μL, 3.00 eq) in DCM (3 mL) was added Ac₂O (42.0 mg, 411 μmol, 38.5 μL, 1.50 eq), the mixture was stirred at 25 °C for 1 h. LCMS showed the desired compound was detected. The reaction mixture was concentrated. The residue was purified by prep-HPLC (column: Waters Xbridge BEH C18 100*30 mm*10 μm; mobile phase: [water (NH₄HCO₃)-ACN]; B %: 30%-70%, 8 min) to give the title compound (Compound 23) (30.6 mg, 74.6 μmol, 27.2% yield, 99.3% purity) as a white solid. LCMS (ES−): m/z 405.0 [M−H]−.

### Example 24. Synthesis of 1-(4-((5-chloro-4-(piperidin-1-yl)pyrimidin-2-yl)amino)piperidin-1-yl)ethan-1-one (Compound 24)

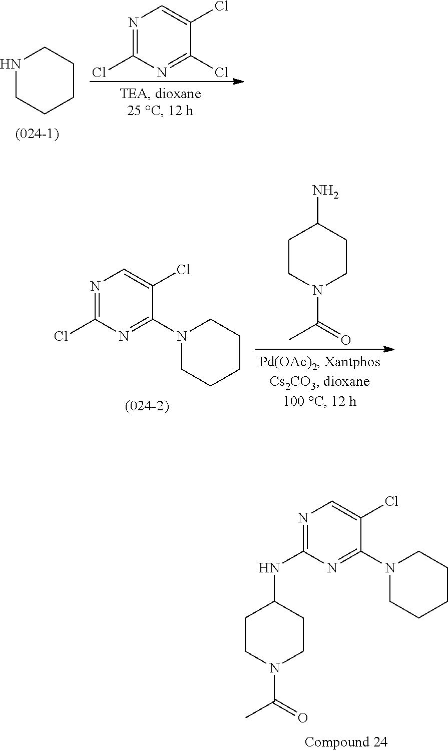

Synthesis of 2,5-dichloro-4-(piperidin-1-yl)pyrimidine (024-2). To a solution of piperidine (024-1) (300 mg, 3.52 mmol, 348 μL, 1.20 eq) in dioxane (6 mL) were added TEA (327 mg, 3.23 mmol, 450 μL, 1.10 eq) and 2,4,5-trichloropyrimidine (539 mg, 2.94 mmol, 1.00 eq) at 25 °C. The mixture was stirred at 25 °C for 12 h. LCMS showed that the desired mass was detected. The reaction mixture was concentrated under reduced pressure to remove solvent. The residue was purified by prep-TLC (SiO₂, Petroleum ether:Ethyl acetate=1:1) to give the title compound (024-2) (455 mg, 1.96 mmol, 66.8% yield) as a white solid. LCMS (ES+): m/z 232.0 [M+H]+.

Synthesis of 1-(4-((5-chloro-4-(piperidin-1-yl)pyrimidin-2-yl)amino)piperidin-1-yl)ethan-1-one (Compound 24). To a solution of 2,5-dichloro-4-(piperidin-1-yl)pyrimidine (024-2) (297 mg, 1.28 mmol, 0.800 eq) in dioxane (10 mL) were added Xantphos (27.8 mg, 48.0 μmol, 0.0300 eq), Cs₂CO₃ (1.04 g, 3.20 mmol, 2.00 eq), Pd(OAc)₂ (18.0 mg, 80.0 mol, 0.0500 eq) and 1-(4-amino-1-piperidyl)ethanone (250 mg, 1.76 mmol, 1.10 eq) at 25 °C under N₂. The mixture was stirred at 100 °C for 12 h under N₂ atmosphere. LCMS showed that 2,5-dichloro-4-(piperidin-1-yl)pyrimidine (024-2) remained little and the desired mass was detected. The reaction mixture was filtered and the filtrate was concentrated under reduced pressure. The residue was purified by prep-HPLC (column: Phenomenex C18 75*30 mm*3 μm; mobile phase: [water (FA)-ACN]; B %: 20%-45%, 8 min) to give the title compound (Compound 24) (9.90 mg, 29.1 μmol, 1.82% yield, 98.4% purity) as a colorless oil. LCMS (ES+): m/z 338.1 [M+H]+.

### Example 25. Synthesis of cis-4-((5-chloro-4-(4-fluorophenyl)pyrimidin-2-yl)amino)cyclohexane-1-carboxamide (Compound 25)

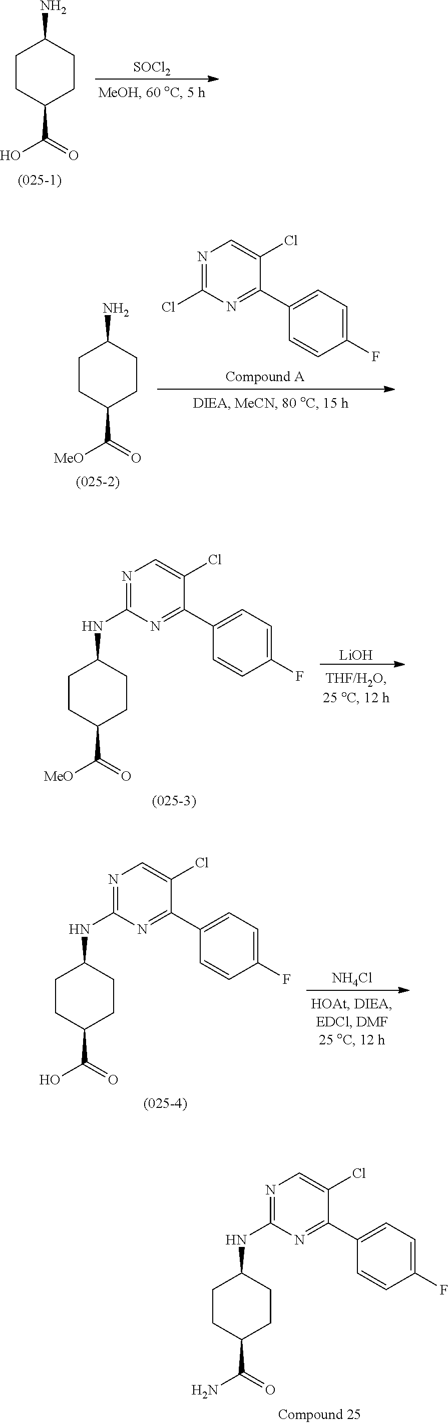

Synthesis of methyl cis-4-aminocyclohexane-1-carboxylate (025-2). To a solution of cis-4-aminocyclohexane-1-carboxylic acid (025-1) (5.00 g, 34.9 mmol, 1.00 eq) in MeOH (120 mL) was added SOCl₂ (8.31 g, 69.8 mmol, 5.07 mL, 2.00 eq) at 25 °C. The mixture was stirred at 60 °C for 5 h. LCMS showed cis-4-aminocyclohexane-1-carboxylic acid (025-1) was consumed completely and the desired mass was detected. The reaction mixture was concentrated under reduced pressure to remove solvent to give the title compound (025-2) (5.56 g, crude) as a white solid. LCMS (ES+): m/z 158.2 [M+H]+.

Synthesis of methyl cis-4-((5-chloro-4-(4-fluorophenyl)pyrimidin-2-yl)amino)cyclohexane-1-carboxylate (025-3). To a solution of methyl cis-4-aminocyclohexane-1-carboxylate (025-2) (3.30 g, 21.0 mmol, 2.50 eq) and 2,5-dichloro-4-(4-fluorophenyl)pyrimidine (2.04 g, 8.40 mmol, 1.00 eq) (prepared by methods known in the art) in MeCN (70 mL) was added DIEA (1.09 g, 8.40 mmol, 1.46 mL, 1.00 eq) at 25 °C. The mixture was stirred at 80 °C for 3 h. LCMS showed ~87% of methyl cis-4-aminocyclohexane-1-carboxylate (025-2) remained and the desired mass was detected. Then the mixture was stirred at 80 °C for 12 h and LCMS showed methyl cis-4-aminocyclohexane-1-carboxylate (025-2) was consumed completely and the desired mass was detected. The reaction mixture was concentrated under reduced pressure to remove solvent. The residue was purified by column chromatography (SiO₂, Ethyl acetate:Petroleum ether=0:1 to 6:10) to give the title compound (025-3) (201 mg, 551 μmol, 6.56% yield) as a yellow oil. LCMS (ES+): m/z 364.1 [M+H]+.

Synthesis of cis-4-((5-chloro-4-(4-fluorophenyl)pyrimidin-2-yl)amino)cyclohexane-1-carboxylic acid (025-4). To a solution of methyl cis-4-((5-chloro-4-(4-fluorophenyl)pyrimidin-2-yl)amino)cyclohexane-1-carboxylate (025-3) (201 mg, 551 mol, 1.00 eq) in THF (9 mL) and H₂O (3 mL) was added LiOH (66.0 mg, 2.76 mmol, 5.00 eq) at 25 °C. The mixture was stirred at 25 °C for 12 h. LCMS showed methyl cis-4-((5-chloro-4-(4-fluorophenyl)pyrimidin-2-yl)amino)cyclohexane-1-carboxylate (025-3) was consumed completely and the desired mass was detected. HCl (1.00 M) was added to the aqueous phase to adjust pH to 2, and the water (8 mL) was added to reaction, the aqueous phase was extracted with EtOAc (8 mL*3), the combined organic layers were dried over Na₂SO₄, filtered and the filtrate was concentrated under reduced pressure to give the title compound (025-4) (418 mg, crude) as a brown solid. LCMS (ES+): m/z 350.2 [M+H]+.

Synthesis of cis-4-((5-chloro-4-(4-fluorophenyl)pyrimidin-2-yl)amino)cyclohexane-1-carboxamide (Compound 25). To a solution of cis-4-((5-chloro-4-(4-fluorophenyl)pyrimidin-2-yl)amino)cyclohexane-1-carboxylic acid (025-4) (418 mg, 1.19 mmol, 1.00 eq) in DMF (4 mL) were added HOAt (293 mg, 2.15 mmol, 301 μL, 1.80 eq), EDCI (412 mg, 2.15 mmol, 1.80 eq), DIEA (463 mg, 3.58 mmol, 624 μL, 3.00 eq) and NH₄Cl (256 mg, 4.78 mmol, 4.00 eq) at 25 °C. The mixture was stirred 25 °C for 12 h. LCMS showed cis-4-((5-chloro-4-(4-fluorophenyl)pyrimidin-2-yl)amino)cyclohexane-1-carboxylic acid (025-4) was consumed completely and the desired mass was detected. The reaction mixture was concentrated under reduced pressure to remove solvent. The residue was purified by neutral prep-HPLC (column: Phenomenex C18 80*40 mm*3 μm; mobile phase: [water (NH₄HCO₃)-ACN]; B %: 30%-60%, 8 min) to give the title compound (Compound 25) (68.2 mg, 196 μmol, 16.4% yield, 100% purity) as a white solid. LCMS (ES+): m/z 349.1 [M+H]+.

### Example 26. Synthesis of 1-(3-((5-chloro-4-(4-fluorophenyl)pyrimidin-2-yl)amino)piperidin-1-yl)ethan-1-one (Compound 26)

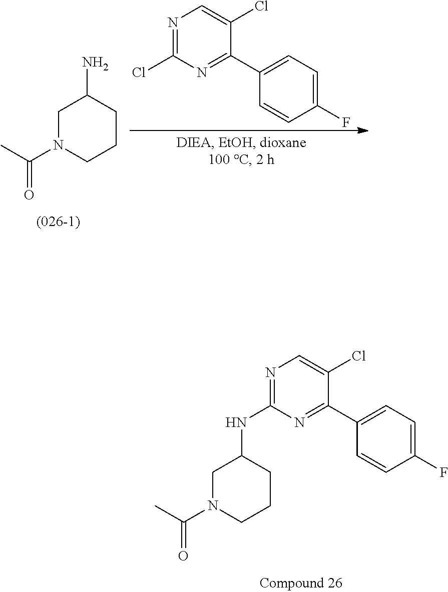

Synthesis of 1-(3-((5-chloro-4-(4-fluorophenyl)pyrimidin-2-yl)amino)piperidin-1-yl)ethan-1-one (Compound 26). To a solution of 2,5-dichloro-4-(4-fluorophenyl)pyrimidine (100 mg, 411 μmol, 1.00 eq) and 1-(3-aminopiperidin-1-yl)ethan-1-one (026-1) (58.5 mg, 411 μmol, 1.00 eq) in EtOH (2 mL) and dioxane (10 mL) was added DIEA (160 mg, 1.23 mmol, 215 μL, 3.00 eq). The mixture was stirred at 100 °C for 2 h. LCMS showed desired MS was detected. The reaction mixture was concentrated under reduced pressure to give a residue. The residue was purified by prep-HPLC (column: Phenomenex luna C18 200*40 mm*10 um; mobile phase: [water (FA)-ACN]; B %: 35%-65%, 8 min) to give the title compound (Compound 26) (30.0 mg, 86.0 μmol, 20.9% yield, 97.03% purity) as a white solid. LCMS (ES+): m/z 349.1[M+H]+.

### Example 27. Synthesis of 1-(4-((5-chloro-4-(4-fluorophenyl)pyrimidin-2-yl)amino)piperidin-1-yl)ethan-1-one (Compound 27)

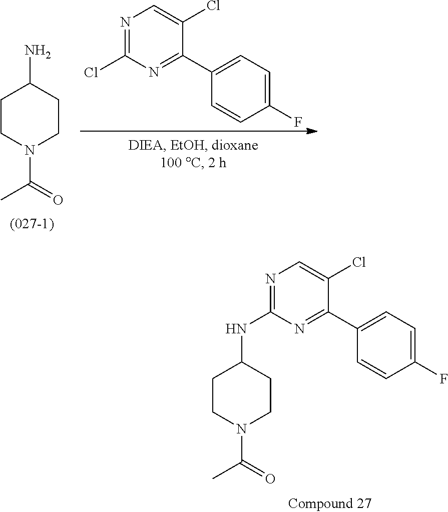

Synthesis of 1-(4-((5-chloro-4-(4-fluorophenyl)pyrimidin-2-yl)amino)piperidin-1-yl)ethan-1-one (Compound 27). To a solution of 2,5-dichloro-4-(4-fluorophenyl)pyrimidine (100 mg, 411 μmol, 1.00 eq) and 1-(4-aminopiperidin-1-yl)ethan-1-one (027-1) (58.5 mg, 411 μmol, 1.00 eq) in dioxane (10 mL) and EtOH (2 mL) was added DIEA (160 mg, 1.23 mmol, 215 μL, 3.00 eq). The mixture was stirred at 100 °C for 2 h. LCMS showed desired MS was detected. The reaction mixture was concentrated under reduced pressure to give a residue. The residue was purified by prep-HPLC (FA condition (column: Phenomenex luna C18 200*40 mm*10 μm; mobile phase: [water (FA)-ACN]; B %: 35%-65%, 8 min) to give the title compound (Compound 27) (33.0 mg, 94.6 μmol, 23.0% yield, 98.95% purity) as a white solid. LCMS (ES+): m/z 349.1[M+H]+.

### Example 28. Synthesis of cis-3-((5-chloro-4-(4-fluorophenyl)pyrimidin-2-yl)amino)cyclohexane-1-carboxamide (Compound 28)

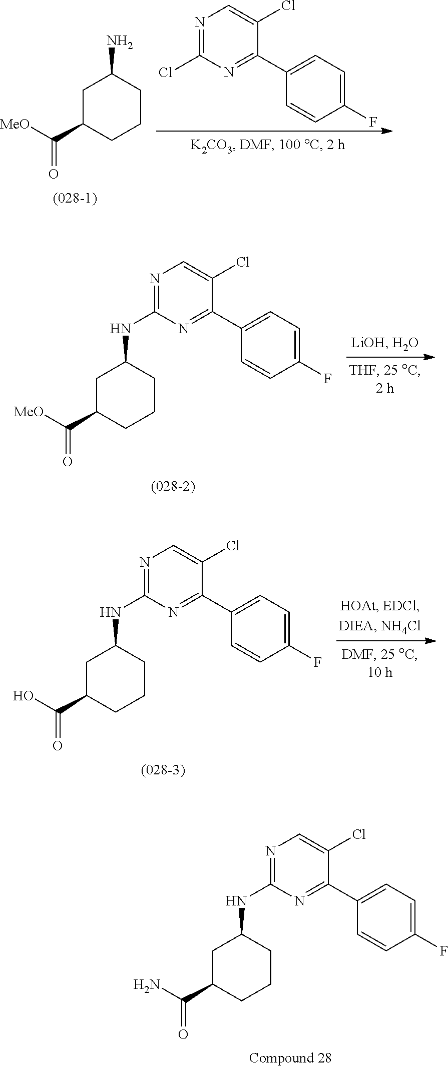

Synthesis of cis-methyl 3-((5-chloro-4-(4-fluorophenyl)pyrimidin-2-yl)amino)cyclohexane-1-carboxylate (028-2). To a solution of cis-methyl 3-aminocyclohexane-1-carboxylate (028-1) (291 mg, 1.85 mmol, 1.00 eq) and 2,5-dichloro-4-(4-fluorophenyl)pyrimidine (450 mg, 1.85 mmol, 1.00 eq) in DMF (5 mL) was added K₂CO₃ (768 mg, 5.55 mmol, 3.00 eq). The mixture was stirred at 100 °C for 2 h. LCMS showed desired MS was detected. The reaction mixture was diluted with H₂O (10 mL) and extracted with ethyl acetate (10 mL*3). The combined organic layers were dried over Na₂SO₄, filtered and concentrated under reduced pressure to give the title compound (028-2) (700 mg, crude) as a blue oil. LCMS (ES+): m/z 364.1 [M+H]+.

Synthesis of cis-3-((5-chloro-4-(4-fluorophenyl)pyrimidin-2-yl)amino)cyclohexane-1-carboxylic acid (028-3). To a solution of cis-methyl 3-((5-chloro-4-(4-fluorophenyl)pyrimidin-2-yl)amino)cyclohexane-1-carboxylate (028-2) (700 mg, 1.92 mmol, 1.00 eq) in THF (2 mL) and H₂O (1 mL) was added LiOH (92.0 mg, 3.84 mmol, 2.00 eq). The mixture was stirred at 25 °C for 2 h. LCMS showed desired MS was detected. The reaction mixture was diluted with H₂O (10 mL), acidified with 1 M of HCl solution to pH=5, and extracted with ethyl acetate (10 mL*3). The combined organic layers were dried over Na₂SO₄, filtered and concentrated under reduced pressure to give the title compound (028-3) (300 mg, crude) as a white solid. LCMS (ES+): m/z 350.1 [M+H]+.

Synthesis of cis-3-((5-chloro-4-(4-fluorophenyl)pyrimidin-2-yl)amino)cyclohexane-1-carboxamide (Compound 28). To a solution of cis-3-((5-chloro-4-(4-fluorophenyl)pyrimidin-2-yl)amino)cyclohexane-1-carboxylic acid (028-3) (270 mg, 772 mol, 1.00 eq) in DMF (3 mL) were added HOAt (126 mg, 926 μmol, 130 μL, 1.20 eq), EDCI (178 mg, 926 μmol, 1.20 eq), DIEA (299 mg, 2.32 mmol, 403 μL, 3.00 eq) and NH₄Cl (82.6 mg, 1.54 mmol, 2.00 eq). The mixture was stirred at 25 °C for 10 h. LCMS showed desired MS was detected. The reaction mixture was concentrated under reduced pressure to give a residue. The residue was purified by prep-HPLC (column: Phenomenex luna C18 80*40 mm*3 μm; mobile phase: [water (FA)-ACN]; B %: 25%-65%, 8 min) to give the title compound (Compound 28) (10.0 mg, 28.7 μmol, 100% yield, 100.00% purity) as a white solid. LCMS (ES+): m/z 349.1 [M+H]+.

### Example 29. Synthesis of 1-((3S)-3-((5-chloro-4-(3-(pyridin-2-yl)pyrrolidin-1-yl)pyrimidin-2-yl)amino)piperidin-1-yl)ethan-1-one (Compound 29)

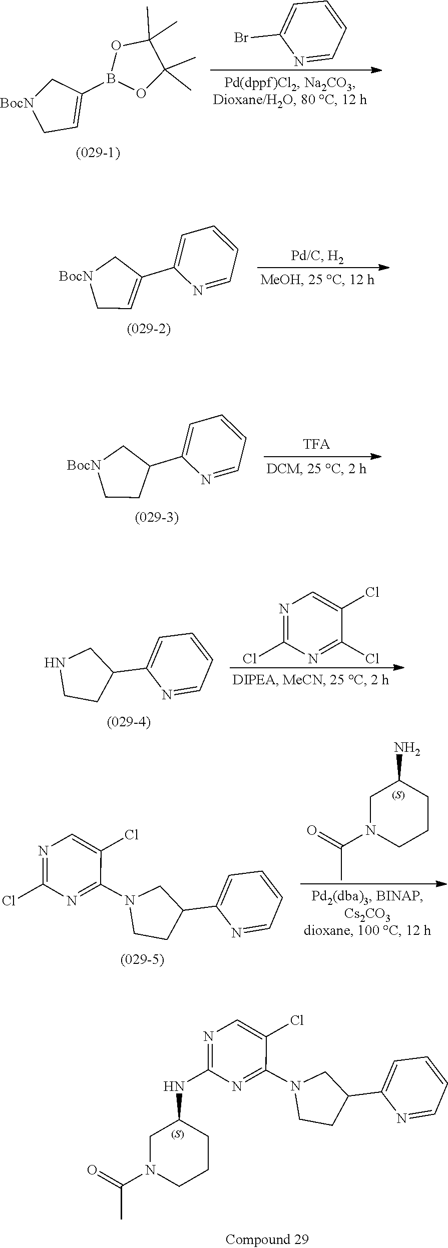

Synthesis of tert-butyl 3-(pyridin-2-yl)-2,5-dihydro-1H-pyrrole-1-carboxylate (029-2). To a solution of tert-butyl 3-(4,4,5,5-tetramethyl-1,3,2-dioxaborolan-2-yl)-2,5-dihydro-1H-pyrrole-1-carboxylate (029-1) (1.00 g, 3.39 mmol, 1.00 eq) (commercially available), 2-bromopyridine (535 mg, 3.39 mmol, 322 μL, 1.00 eq), Na₂CO₃ (718 mg, 6.78 mmol, 2.00 eq) in dioxane (150 mL) and H₂O (150 mL) was degassed and purged with N₂ for 3 times, and then the mixture was added Pd(dppf)Cl₂ (247 mg, 338 μmol, 0.100 eq) at 25 °C. The mixture was stirred at 80 °C for 12 h under N₂ atmosphere. LCMS showed tert-butyl 3-(4,4,5,5-tetramethyl-1,3,2-dioxaborolan-2-yl)-2,5-dihydro-1H-pyrrole-1-carboxylate (029-1) was consumed completely and the desired mass was detected. H₂O 20 mL was added to the mixture, and extracted with DCM (20 mL*3). The combined organic layers were dried over Na₂SO₄, filtered and concentrated under reduced pressure. The residue was purified by column chromatography (SiO₂, Petroleum ether/Ethyl acetate=20/1 to 1/1) to give the title compound (029-2) (1.9 g, 7.71 mmol, 75.9% yield) as a colorless oil. LCMS (ES+): m/z 191.2 [M+H−56]+.

Synthesis of tert-butyl 3-(pyridin-2-yl)pyrrolidine-1-carboxylate (029-3). To a solution of tert-butyl 3-(pyridin-2-yl)-2,5-dihydro-1H-pyrrole-1-carboxylate (029-2) (1.00 g, 4.06 mmol, 1.00 eq) in MeOH (8 mL) was degassed and purged with H₂ (15 psi) for 3 times, then the 10% Pd/C (1.00 g, 50% purity) was added to the mixture at 25 °C. The mixture was stirred at 25 °C for 12 h under H₂ (15 psi) atmosphere. LCMS showed tert-butyl 3-(pyridin-2-yl)-2,5-dihydro-1H-pyrrole-1-carboxylate (029-2) was consumed completely and the desired mass was detected. The reaction mixture was filtered and the filtrate concentrated under high vacuum to give the title compound (029-3) (1 g, crude) as a yellow oil. LCMS (ES+): m/z 193.2 [M+H−56]+.

Synthesis of 2-(pyrrolidin-3-yl)pyridine (029-4). A mixture of tert-butyl 3-(pyridin-2-yl)pyrrolidine-1-carboxylate (029-3) (1 g, 4.03 mmol, 1.00 eq) in DCM (4 mL) and TFA (1.54 g, 13.5 mmol, 1 mL, 3.35 eq) was stirred at 25 °C for 2 h. LCMS showed tert-butyl 3-(pyridin-2-yl)pyrrolidine-1-carboxylate (029-3) was consumed completely and the desired mass was detected. The reaction mixture was concentrated under high vacuum to give the title compound (029-4) (520 mg, crude) as a yellow oil. LCMS (ES+): m/z 149.4 [M+H]+.

Synthesis of 2,5-dichloro-4-(3-(pyridin-2-yl)pyrrolidin-1-yl)pyrimidine (029-5). To a solution of 2-(pyrrolidin-3-yl)pyridine (029-4) (500 mg, 3.37 mmol, 1.00 eq) and 2,4,5-trichloropyrimidine (618 mg, 3.37 mmol, 1.00 eq) in ACN (2 mL) was added DIEA (872 mg, 6.75 mmol, 1.18 mL, 2.00 eq) at 25 °C, The mixture was stirred at 25 °C for 2 h. LCMS showed 2-(pyrrolidin-3-yl)pyridine (029-4) was consumed completely and the desired mass was detected. The reaction mixture was quenched by addition H₂O 5 mL, and extracted with DCM (10 mL*3). The combined organic layers were dried over Na₂SO₄, filtered and concentrated under reduced pressure. The residue was purified by column chromatography (SiO₂, Petroleum ether/Ethyl acetate=20/1 to 1/1) to give the title compound (029-5) (680 mg, 2.30 mmol, 68.2% yield) as a white solid. LCMS (ES+): m/z 295.2[M+H]+.

Synthesis of 1-((3S)-3-((5-chloro-4-(3-(pyridin-2-yl)pyrrolidin-1-yl)pyrimidin-2-yl)amino)piperidin-1-yl)ethan-1-one (Compound 29). To a solution of 2,5-dichloro-4-(3-(pyridin-2-yl)pyrrolidin-1-yl)pyrimidine (029-5) (200 mg, 677 μmol, 1.00 eq), (S)-1-(3-aminopiperidin-1-yl)ethan-1-one (Compound C) (134 mg, 948 μmol, 1.40 eq), Cs₂CO₃ (551 mg, 1.69 mmol, 2.50 eq), BINAP (42.1 mg, 67.7 μmol, 0.100 eq) in dioxane (5 mL) was added Pd₂(dba)₃ (62.0 mg, 67.7 μmol, 0.100 eq) at 25 °C under N₂. The mixture was stirred at 100 °C for 12 h. LCMS showed 2,5-dichloro-4-(3-(pyridin-2-yl)pyrrolidin-1-yl)pyrimidine (029-5) was consumed completely and the desired mass was detected. H₂O 10 mL was added to the mixture, and extracted with EtOAc (10 mL*3). The combined organic layers were dried over Na₂SO₄, filtered and concentrated under reduced pressure. The residue was purified by prep-HPLC (column: Waters Xbridge BEH C18 100*30 mm*10 μm; mobile phase: [water(NH₄HCO₃)-ACN]; B %: 40%-70%, 8 min) to give the title compound (Compound 29) (40.3 mg, 99.7 μmol, 14.7% yield, 99.2% purity) as a white solid. LCMS (ES+): m/z 401.2 [M+H]+.

### Example 30. Synthesis of 1-(4-((5-chloro-4-(1-phenylpyrrolidin-3-yl)pyrimidin-2-yl)amino)piperidin-1-yl)ethan-1-one (Compound 30)

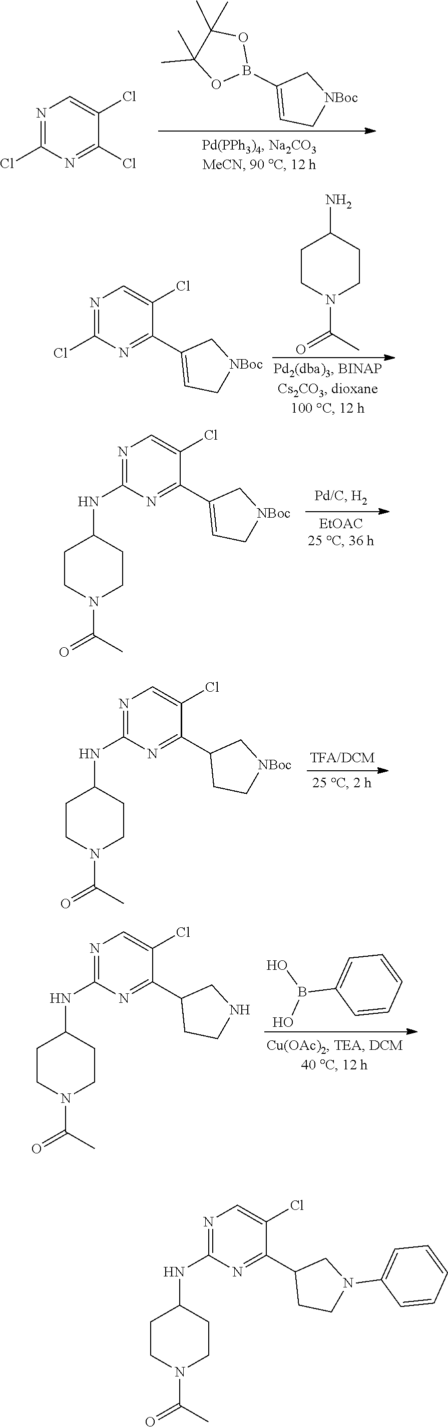

Synthesis of tert-butyl 3-(2,5-dichloropyrimidin-4-yl)-2,5-dihydro-1H-pyrrole-1-carboxylate. To a solution of tert-butyl 3-(4,4,5,5-tetramethyl-1,3,2-dioxaborolan-2-yl)-2,5-dihydropyrrole-1-carboxylate (4.00 g, 13.55 mmol, 1.00 eq) and 2,4,5-trichloropyrimidine (2.98 g, 16.26 mmol, 1.20 eq) in dioxane (40 mL) and H₂O (4 mL) was added Pd(PPh₃)₄ (782.95 mg, 677.55 μmol, 0.0500 eq) and Na₂CO₃ (2.87 g, 27.10 mmol, 2.00 eq) at 25 °C under N₂ atmosphere. The mixture was stirred at 80 °C for 12 h under N₂ atmosphere. LCMS showed the desired mass was detected. The mixture was concentrated in vacuo, diluted with H₂O (100 mL) and then extracted with EtOAc (20 mL*3). The combined organic layers were dried over Na₂SO₄, filtered and the filtrate was concentrated in vacuo. The residue was purified by column chromatography (SiO₂, Petroleum ether/Ethyl acetate=1/0 to 5/1) to give tert-butyl 3-(2,5-dichloropyrimidin-4-yl)-2,5-dihydropyrrole-1-carboxylate (3.02 g, 9.55 mmol, 70.49% yield) as a yellow oil. LCMS (ES+): m/z 339.1 [M+Na]+.

Synthesis of tert-butyl 3-(2-((1-acetylpiperidin-4-yl)amino)-5-chloropyrimidin-4-yl)-2,5-dihydro-1H-pyrrole-1-carboxylate). To a solution of tert-butyl 3-(2,5-dichloropyrimidin-4-yl)-2,5-dihydro-1H-pyrrole-1-carboxylate (400 mg, 1.27 mmol, 1.00 eq) and 1-(4-aminopiperidin-1-yl)ethan-1-one (180 mg, 1.27 mmol, 1.00 eq) in dioxane (5 mL) was added Cs₂CO₃ (824 mg, 2.53 mmol, 2.00 eq), Pd₂(dba)₃ (69.5 mg, 75.9 μmol, 0.0600 eq) and BINAP (31.5 mg, 50.6 μmol, 0.0400 eq) at 25 °C. The mixture was stirred at 100 °C for 12 h under N₂ atmosphere. LCMS showed tert-butyl 3-(2,5-dichloropyrimidin-4-yl)-2,5-dihydro-1H-pyrrole-1-carboxylate remained and desired mass was detected. The mixture was concentrated in vacuo. The mixture was diluted with H₂O (20 mL) and then extracted with EtOAc (6 mL*3). The combined organic layers were dried over Na₂SO₄, filtered and the filtrate was concentrated in vacuo. The residue was purified by prep-TLC (SiO₂, Petroleum ether/Ethyl acetate=1/5) to give the title compound (251 mg, 595 μmol, 47.0% yield) as a white solid. LCMS (ES+): m/z 422.1 [M+H]⁺.

Synthesis of tert-butyl 3-(2-((1-acetylpiperidin-4-yl)amino)-5-chloropyrimidin-4-yl)pyrrolidine-1-carboxylate. To a stirred solution of tert-butyl 3-(2-((1-acetylpiperidin-4-yl)amino)-5-chloropyrimidin-4-yl)-2,5-dihydro-1H-pyrrole-1-carboxylate (100 mg, 237 μmol, 1.00 eq) in EtOAc (4 mL) was added 10% Pd/C (100 mg, 50% purity) at 25 °C under N₂. The resulting mixture was stirred at 25 °C for 0.5 h under H₂ (15 psi). LCMS showed tert-butyl 3-(2-((1-acetylpiperidin-4-yl)amino)-5-chloropyrimidin-4-yl)-2,5-dihydro-1H-pyrrole-1-carboxylate remained. The resulting mixture was stirred at 25 °C for 12 h under H₂ (15 psi). LCMS showed tert-butyl 3-(2-((1-acetylpiperidin-4-yl)amino)-5-chloropyrimidin-4-yl)-2,5-dihydro-1H-pyrrole-1-carboxylate remained and the desired mass was detected. The mixture was filtered and to the filtrate was added 10% Pd/C (100 mg, 50% purity). The resulting mixture was stirred at 25 °C for 8 h under H₂ (15 psi). LCMS showed tert-butyl 3-(2-((1-acetylpiperidin-4-yl)amino)-5-chloropyrimidin-4-yl)-2,5-dihydro-1H-pyrrole-1-carboxylate remained and the desired mass was detected. The resulting mixture was stirred at 25 °C for 3 h under H₂ (15 psi). LCMS showed tert-butyl 3-(2-((1-acetylpiperidin-4-yl)amino)-5-chloropyrimidin-4-yl)-2,5-dihydro-1H-pyrrole-1-carboxylate was consumed mostly and the desired mass was detected. The resulting mixture was stirred at 25 °C for 12 h under H₂ (15 psi). LCMS showed tert-butyl 3-(2-((1-acetylpiperidin-4-yl)amino)-5-chloropyrimidin-4-yl)-2,5-dihydro-1H-pyrrole-1-carboxylate was consumed mostly and the desired mass was detected. The mixture was concentrated in vacuo to give the title compound (100 mg, crude) as a yellow solid. LCMS (ES+): m/z 424.1 [M+H]+.

Synthesis of 1-(4-((5-chloro-4-(pyrrolidin-3-yl)pyrimidin-2-yl)amino)piperidin-1-yl)ethan-1-one. To a solution of tert-butyl 3-(2-((1-acetylpiperidin-4-yl)amino)-5-chloropyrimidin-4-yl)pyrrolidine-1-carboxylate (60 mg, 142 μmol, 1.00 eq) in DCM (1 mL) was added TFA (0.33 mL) at 25 °C. The mixture was stirred at 25 °C for 1 h. LCMS shows tert-butyl 3-(2-((1-acetylpiperidin-4-yl)amino)-5-chloropyrimidin-4-yl)pyrrolidine-1-carboxylate was consumed and the desired mass was detected. The reaction mixture was concentrated under reduced pressure to remove solvent to give the title compound (60 mg, crude, TFA) as a yellow oil. LCMS (ES+): m/z 324.1 [M+H]+.

Synthesis of 1-(4-((5-chloro-4-(1-phenylpyrrolidin-3-yl)pyrimidin-2-yl)amino)piperidin-1-yl)ethan-1-one (Compound 30). To a solution of 1-(4-((5-chloro-4-(pyrrolidin-3-yl)pyrimidin-2-yl)amino)piperidin-1-yl)ethan-1-one (40.0 mg, 91.4 μmol, 1.00 eq, TFA) and phenylboronic acid (22.3 mg, 183 μmol, 2.00 eq) in DCM (1 mL) were added Cu(OAc)₂ (16.6 mg, 91.4 μmol, 1.00 eq), TEA (27.7 mg, 274 μmol, 38.15 μL, 3.00 eq) and 4AMS (10.0 mg, 91.4 μmol, 1.00 eq) at 25 °C. The mixture was stirred at 40 °C for 12 h under OXYGEN (15 psi). LCMS showed 1-(4-((5-chloro-4-(pyrrolidin-3-yl)pyrimidin-2-yl)amino)piperidin-1-yl)ethan-1-one was remained and desired mass was detected. The mixture was concentrated in vacuo. The mixture was diluted with H₂O (20 mL) and then extracted with DCM (5 mL*3). The combined organic layers were dried over Na₂SO₄, filtered and the filtrate was concentrated in vacuo. The residue was purified by prep-HPLC (column: Phenomenex C18 80 * 40 mm*3 μm; mobile phase: [water (NH₄HCO₃)-ACN]; B %: 35%-65%, 8 min) to give the title compound (Compound 30) (8.70 mg, 21.5 μmol, 23.5% yield, 98.6% purity) as a yellow solid. LCMS (ES+): m/z 400.1 [M+H]+.

### Example 31. Synthesis of N-(cis-4-((4-([1,1′-biphenyl]-3-yl)-5-chloropyrimidin-2-yl)amino)cyclohexyl)acetamide (Compound 31)

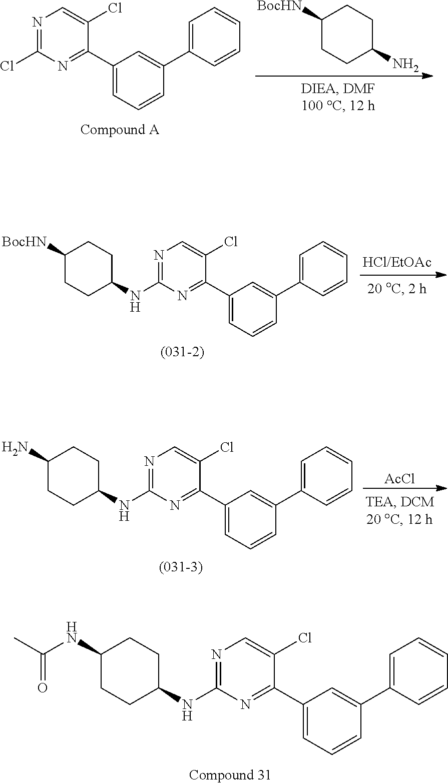 [SMILES: Clc1ncc(Cl)c(-c2cccc(-c3ccccc3)c2)n1 ; CC(C)(C)OC(=O)N[C@H]1CC[C@@H](N)CC1 ; "AcCl" ; CC(C)(C)OC(=O)N[C@H]1CC[C@@H](Nc2ncc(Cl)c(-c3cccc(-c4ccccc4)c3)n2)CC1 ; N[C@H]1CC[C@@H](Nc2ncc(Cl)c(-c3cccc(-c4ccccc4)c3)n2)CC1 ; CC(=O)N[C@H]1CC[C@@H](Nc2ncc(Cl)c(-c3cccc(-c4ccccc4)c3)n2)CC1]

Synthesis of tert-butyl (cis-4-((4-([1,1′-biphenyl]-3-yl)-5-chloropyrimidin-2-yl)amino)cyclohexyl)carbamate (031-2). To a solution of 2,5-dichloro-4-(3-phenylphenyl)pyrimidine (Compound A) (300 mg, 996 μmol, 1.00 eq) in DMF (3 mL) was added DIEA (515 mg, 3.98 mmol, 694 μL, 4.00 eq) and cis-tert-butyl N-(4-aminocyclohexyl)carbamate (235 mg, 1.10 mmol, 1.10 eq) at 20 °C. The mixture was stirred at 100 °C for 12 h. LCMS showed that 2,5-dichloro-4-(3-phenylphenyl)pyrimidine (Compound A) (031-2) was consumed completely and one main peak with the desired mass was detected. Water (10 mL) was added to the mixture, then it was extracted with EtOAc (3 mL*3). The combined organic layers were washed with brine (5 mL), dried over Na₂SO₄, filtered and concentrated under reduced pressure. The residue was purified by prep-TLC (SiO₂, Petroleum ether/Ethyl acetate=3/1) to give the title compound (031-2) (480 mg, crude) as a yellow oil. LCMS (ES+): m/z 479.2 [M+H]+.

Synthesis of cis-N1-(4-([1,1′-biphenyl]-3-yl)-5-chloropyrimidin-2-yl)cyclohexane-1,4-diamine (031-3). A solution of tert-butyl (cis-4-((4-([1,1′-biphenyl]-3-yl)-5-chloropyrimidin-2-yl)amino)cyclohexyl)carbamate (031-2) (400 mg, 835 μmol, 1.00 eq) in HCl/EtOAc (4 M, 16.00 mL) was stirred at 20 °C for 2 h. LCMS showed that tert-butyl (cis-4-((4-([1,1′-biphenyl]-3-yl)-5-chloropyrimidin-2-yl)amino)cyclohexyl)carbamate (031-2) was consumed completely and one main peak with the desired mass was detected. The mixture was concentrated to give the title compound (031-3) (350 mg, crude, HCl) as a white solid. LCMS (ES+): m/z 379.1 [M+H]+.

Synthesis of N-(cis-4-((4-([1,1′-biphenyl]-3-yl)-5-chloropyrimidin-2-yl)amino)cyclohexyl)acetamide (Compound 31). To a solution of cis-N1-(4-([1,1′-biphenyl]-3-yl)-5-chloropyrimidin-2-yl)cyclohexane-1,4-diamine (031-3) (200 mg, 482 μmol, 1.00 eq, HCl) in DCM (2 mL) were added TEA (146 mg, 1.44 mmol, 201 μL, 3.00 eq) and acetyl chloride (18.9 mg, 241 μmol, 17.2 μL, 0.500 eq) at 20 °C. The mixture was stirred at 20 °C for 2 h. LCMS showed that cis-N1-(4-([1,1′-biphenyl]-3-yl)-5-chloropyrimidin-2-yl)cyclohexane-1,4-diamine (031-3) remained and one main peak with the desired mass was detected. The mixture was concentrated. The residue was dissolved in MeCN (4 mL). The suspension was filtered and the filtrate was purified by acidic prep-HPLC (column: Phenomenex Luna C18 200*40 mm*10 um; mobile phase: [water (FA)-ACN]; B %: 45%-90%, 8 min). The fraction was concentrated under reduced pressure to remove most of MeCN at 30 °C and the aqueous phase was lyophilized to give the title compound (Compound 31) (117 mg, 275 μmol, 57.0% yield, 99.2% purity) as a white solid. LCMS (ES+): m/z 421.1 [M+H]+.

### Example 32. Synthesis of N-(trans-4-((4-([1,1′-biphenyl]-3-yl)-5-chloropyrimidin-2-yl)amino)cyclohexyl)acetamide (Compound 32)

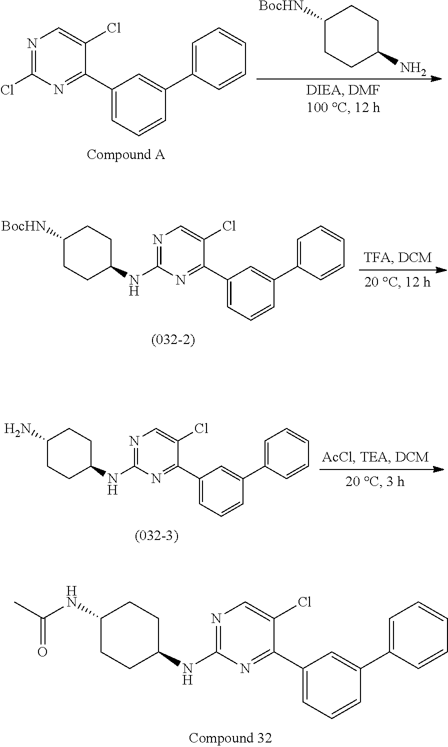 [SMILES: Clc1ncc(Cl)c(-c2cccc(-c3ccccc3)c2)n1 ; CC(C)(C)OC(=O)N[C@H]1CC[C@H](N)CC1 ; CC(C)(C)OC(=O)N[C@H]1CC[C@H](Nc2ncc(Cl)c(-c3cccc(-c4ccccc4)c3)n2)CC1 ; N[C@H]1CC[C@H](Nc2ncc(Cl)c(-c3cccc(-c4ccccc4)c3)n2)CC1 ; CC(=O)N[C@H]1CC[C@H](Nc2ncc(Cl)c(-c3cccc(-c4ccccc4)c3)n2)CC1]

Synthesis of tert-butyl (trans-4-((4-([1,1′-biphenyl]-3-yl)-5-chloropyrimidin-2-yl)amino)cyclohexyl)carbamate (032-2). To a solution of 2,5-dichloro-4-(3-phenylphenyl)pyrimidine (Compound A) (300 mg, 996 μmol, 1.00 eq) and trans-N-Boc-1,4-cyclohexanediamine hydrochloride (235 mg, 1.10 mmol, 1.10 eq) in DMF (3 mL) was added DIEA (515 mg, 3.98 mmol, 694 μL, 4.00 eq) at 20 °C. The mixture was stirred at 100 °C for 12 h. LCMS showed 2,5-dichloro-4-(3-phenylphenyl)pyrimidine (Compound A) was consumed completely and one main peak with desired mass was detected. Water (10 mL) was added to the mixture, then it was extracted with EtOAc (3 mL*3). The combined organic layers were washed with brine (5 mL), dried over Na₂SO₄, filtered and concentrated under reduced pressure. The residue was purified by prep-TLC (SiO₂, Petroleum ether/Ethyl acetate=3/1) to give the title compound (032-2) (480 mg, crude) as a yellow solid. LCMS (ES+): m/z 479.2 [M+H]+.

Synthesis of trans-N1-(4-([1,1′-biphenyl]-3-yl)-5-chloropyrimidin-2-yl)cyclohexane-1,4-diamine (032-3). To a solution of tert-butyl (trans-4-((4-([1,1′-biphenyl]-3-yl)-5-chloropyrimidin-2-yl)amino)cyclohexyl)carbamate (032-2) (480 mg, 1.00 mmol, 1.00 eq) in DCM (5 mL) was added TFA (114 mg, 1.00 mmol, 74.2 μL, 1.00 eq) at 20 °C. The mixture was stirred at 20 °C for 12 h. LCMS showed that tert-butyl (trans-4-((4-([1,1′-biphenyl]-3-yl)-5-chloropyrimidin-2-yl)amino)cyclohexyl)carbamate (032-2) was consumed completely and one peak with desired mass was detected. The mixture was concentrated to give the title compound (032-3) (450 mg, crude, TFA) as a brown oil. LCMS (ES−): m/z 377.3 [M−H]−.

Synthesis of N-(trans-4-((4-([1,1′-biphenyl]-3-yl)-5-chloropyrimidin-2-yl)amino)cyclohexyl)acetamide (Compound 32). To a solution of trans-N1-(4-([1,1-biphenyl]-3-yl)-5-chloropyrimidin-2-yl)cyclohexane-1,4-diamine (032-3) (200 mg, 406 μmol, 1.00 eq, TFA) in DCM (2 mL) were added TEA (205 mg, 2.03 mmol, 282 μL, 4.99 eq) and acetyl chloride (12.7 mg, 162 μmol, 11.6 μL, 0.40 eq) at 20 °C. The mixture was stirred at 20 °C for 3 h. LCMS showed that trans-N1-(4-([1,1′-biphenyl]-3-yl)-5-chloropyrimidin-2-yl)cyclohexane-1,4-diamine (032-3) was consumed completely and one main peak with desired mass was detected. The mixture was concentrated. The residue was dissolved in DMSO (3 mL). The suspension was filtered and the filtrate was purified by prep-HPLC (column: Phenomenex Luna C18 75*30 mm*3 μm; mobile phase: [water (FA)-ACN]; B %: 30%-60%, 8 min). The fraction was concentrated under reduced pressure to remove most of MeCN at 30 °C and the aqueous phase was lyophilized to give the title compound (Compound 32) (41.1 mg, 96.4 μmol, 23.8% yield, 98.7% purity) as a white solid. LCMS (ES+): m/z 421.1 [M+H]+.

### Example 33. Synthesis of cis-N-(3-((4-([1,1′-biphenyl]-3-yl)-5-chloropyrimidin-2-yl)amino)cyclohexyl)acetamide (Compound 33)

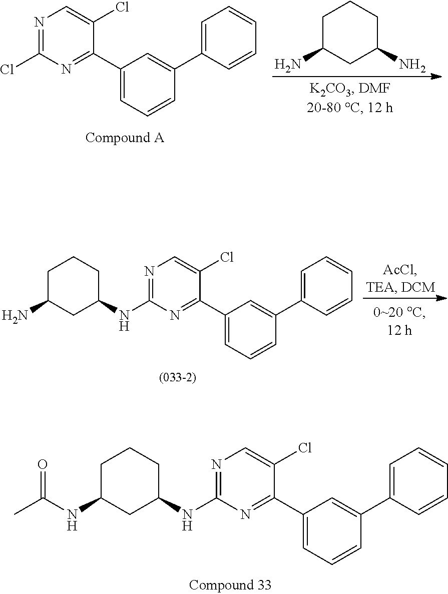

Synthesis of cis-NJ-(4-([1,1′-biphenyl]-3-yl)-5-chloropyrimidin-2-yl)cyclohexane-1,3-diamine (033-2). To a solution of 2,5-dichloro-4-(3-phenylphenyl)pyrimidine (Compound A) (200 mg, 664 μmol, 1.00 eq) and (1R,3S)-cyclohexane-1,3-diamine (379 mg, 3.32 mmol, 5.00 eq) in DMF (4 mL) was added K₂CO₃ (184 mg, 1.33 mmol, 2.00 eq) at 20 °C. The mixture was stirred at 80 °C for 12 h. LCMS showed that the desired compound was detected. H₂O (20 mL) was added to the mixture, the mixture was extracted with methylene dichloride/methyl alcohol=10/1 (15 mL*3), the combined organic layers were dried over Na₂SO₄, filtered and concentrated under reduced pressure. The residue was purified by prep-TLC (SiO₂, Ethyl acetate:Petroleum ether=3:1) to give the title compound (033-2) (114 mg, 301 μmol, 45.3% yield) as a yellow oil. LCMS (ES+): m/z 379.1 [M+H]+.

Synthesis of cis-N-(3-((4-([1,1′-biphenyl]-3-yl)-5-chloropyrimidin-2-yl)amino)cyclohexyl)acetamide (Compound 33). To a solution of rac-N1-[5-chloro-4-(3-phenylphenyl)pyrimidin-2-yl]cyclohexane-1,3-diamine (033-2) (114 mg, 301 μmol, 1.00 eq) in DCM (1 mL) was added TEA (91.3 mg, 902 μmol, 126 μL, 3.00 eq) and acetyl chloride (23.6 mg, 301 μmol, 21.5 μL, 1.00 eq) at 0 °C. The mixture was stirred at 20 °C for 12 h. LCMS showed that the desired compound was detected. The reaction mixture was concentrated under reduced pressure. The residue was purified by prep-HPLC (column: Phenomenex Luna C18 200*40 mm*10 μm; mobile phase: [water (FA)-ACN]; B %: 45%-80%, 8 min) to give the title compound (Compound 33) (8.3 mg, 19.7 μmol, 6.55% yield, 100% purity) as a white solid. LCMS (ES+): m/z 421.1 [M+H]+.

### Example 34. tras-N-(3-((4-([1,1′-biphenyl]-3-yl)-5-chloropyrimidin-2-yl)amino)cyclohexyl)acetamide (Compound 34)

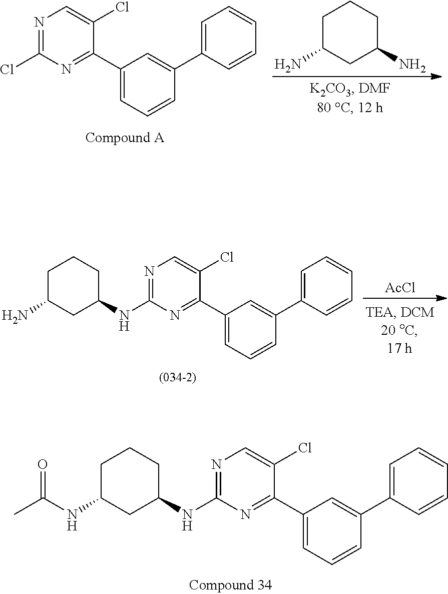

Synthesis of trans-N1-(4-([1,1′-biphenyl]-3-yl)-5-chloropyrimidin-2-yl)cyclohexane-1,3-diamine (034-2). To a solution of 4-([1,1′-biphenyl]-3-yl)-2,5-dichloropyrimidine (Compound A) (200 mg, 664 μmol, 1.00 eq) in DMF (4 mL) were added K₂CO₃ (184 mg, 1.33 mmol, 2.00 eq) and trans-cyclohexane-1,3-diamine (91.0 mg, 797 μmol, 1.20 eq) at 20 °C. The mixture was stirred at 80 °C for 12 h. LCMS showed 4-([1,1′-biphenyl]-3-yl)-2,5-dichloropyrimidine (Compound A) (034-1) was consumed completely and the desired mass was detected. The mixture was diluted with H₂O (10 mL) and extracted with EtOAc (3 mL * 3). The organic layers were washed with brine (15 mL) dried over Na₂SO₄, filtered and concentrated under reduced pressure. The residue was purified by prep-TLC (SiO₂, Petroleum ether:Ethyl acetate=3:1) to give title compound (034-2) (150 mg, 396 μmol, 59.6% yield) as brown oil. LCMS (ES+): m/z 379.1 [M+H]+.

Synthesis of trans-N-(3-((4-([1,1′-biphenyl]-3-yl)-5-chloropyrimidin-2-yl)amino)cyclohexyl)acetamide (Compound 34). To a solution of trans-N1-(4-([1,1′-biphenyl]-3-yl)-5-chloropyrimidin-2-yl)cyclohexane-1,3-diamine (034-2) (130 mg, 343 μmol, 1.00 eq) and acetyl chloride (8.08 mg, 103 μmol, 7.35 μL, 0.300 eq) in DCM (3 mL) was added Py (81.4 mg, 1.03 mmol, 83.1 μL, 3.00 eq) in DCM (1 mL) at 20 °C. The mixture was stirred at 20 °C for 12 h. LCMS showed that trans-N1-(4-([1,1′-biphenyl]-3-yl)-5-chloropyrimidin-2-yl)cyclohexane-1,3-diamine (034-2) remained and the desired mass was detected. Acetyl chloride (10.8 mg, 137 μmol, 9.76 μL, 0.400 eq) was added to the mixture at 20 °C. The mixture was stirred at 20 °C for 5 h. LCMS showed most of trans-N1-(4-([1,1′-biphenyl]-3-yl)-5-chloropyrimidin-2-yl)cyclohexane-1,3-diamine (034-2) was consumed and the desired mass was detected. The mixture was concentrated under reduced pressure. The residue was purified by prep-HPLC (column: C18-6 100*30 mm*5 μm; mobile phase: [water (FA)-ACN]; B %: 50%-80%, 8 min) to give title compound (Compound 34) (18.2 mg, 42.8 μmol, 12.5% yield, 99.0% purity) as a white solid. LCMS (ES+): m/z 421.1 [M+H]+.

### Example 35. Synthesis of 1-((3S)-3-((5-chloro-4-(3-phenylpiperidin-1-yl) pyrimidin-2-yl) amino)piperidin-1-yl) ethan-1-one (Compound 35)

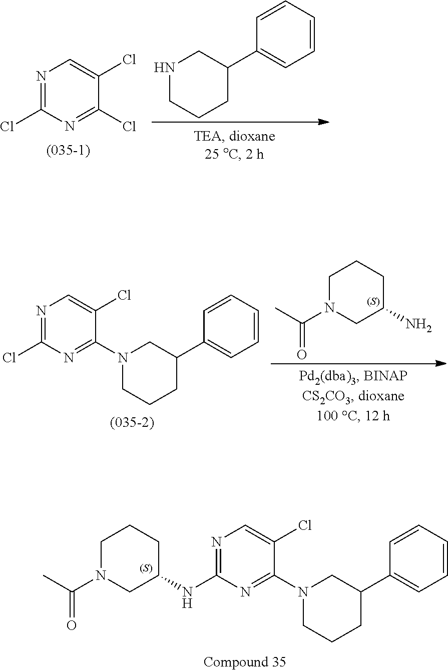

Synthesis of 2, 5-dichloro-4-(3-phenylpiperidin-1-yl) pyrimidine (035-2). To a solution of 2,4,5-trichloropyrimidine (035-1) (300 mg, 1.64 mmol, 1.00 eq) and 3-phenylpiperidine (264 mg, 1.64 mmol, 1.00 eq) in dioxane (3 mL) was added TEA (331 mg, 3.27 mmol, 455 μL, 2.00 eq) at 25 °C, the mixture was stirred at 25 °C for 1 h. LCMS showed the desired target MS was detected. The mixture was concentrated in vacuo, then H₂O (8 mL) was added to the mixture and the mixture was extracted with EtOAc (8 mL*3), the combined organic layers were washed with saturated aqueous NaCl solution (5 mL*3), dried over Na₂SO₄, filtered and the filtrate was concentrated in vacuo. The mixture was purified by prep-TLC (SiO₂, PE/EtOAc=20/1) to give the title compound (035-2) (360 mg, 1.17 mmol, 71.4% yield) as a colorless oil. LCMS (ES+): m/z 308.0 [M+H]+.

Synthesis of 1-((3S)-3-((5-chloro-4-(3-phenylpiperidin-1-yl) pyrimidin-2-yl) amino) piperidin-1-yl) ethan-1-one (Compound 35). To a solution of 2, 5-dichloro-4-(3-phenylpiperidin-1-yl) pyrimidine (035-2) (270 mg, 876 μmol, 1.00 eq) and (S)-1-(3-aminopiperidin-1-yl)ethan-1-one (Compound C) (125 mg, 876 μmol, 1.00 eq) in dioxane (4 mL) were added BINAP (32.7 mg, 52.6 μmol, 0.0600 eq), Cs₂CO₃ (571 mg, 1.75 mmol, 2.00 eq) and Pd₂(dba)₃ (32.1 mg, 35.0 μmol, 0.0400 eq) at 25 °C, the mixture was stirred at 100 °C for 12 h under N₂ atmosphere, LCMS showed 2, 5-dichloro-4-(3-phenylpiperidin-1-yl) pyrimidine (035-2) was consumed, desired target MS was detected. The mixture was concentrated in vacuo, then H₂O (10 mL) was added to the mixture and the mixture was extracted with EtOAc (10 mL*3), the combined organic layers were washed with saturated aqueous NaCl solution (5 mL*3), dried over Na₂SO₄, filtered and the filtrate was concentrated in vacuo. The crude product was purified by prep-HPLC (column: Phenomenex Luna C18 200*40 mm*10 μm; mobile phase: [water (FA)-ACN]; B %: 25%-60%, 8 min) to give the title compound (Compound 35) (72.1 mg, 170 μmol, 19.4% yield, 97.5% purity) as a white solid. LCMS (ES+): m/z 414.2 [M+H]+.

### Example 36. Synthesis of cis-3-((5-fluoro-4-(3-(pyrrolidin-1-yl)phenyl)pyrimidin-2-yl)amino)cyclohexane-1-carboxamide (Compound 36)

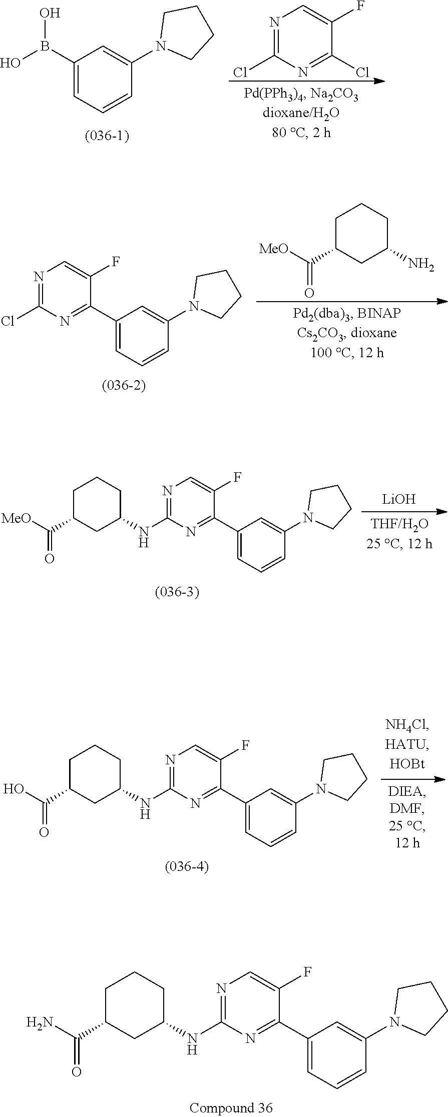

Synthesis of 2-chloro-5-fluoro-4-(3-(pyrrolidin-1-yl)phenyl)pyrimidine (036-2). To a stirred solution of (3-(pyrrolidin-1-yl)phenyl)boronic acid (036-1) (300 mg, 1.57 mmol, 1.00 eq) and 2,4-dichloro-5-fluoropyrimidine (393 mg, 2.36 mmol, 1.50 eq) in dioxane (5 mL) and H₂O (0.5 mL) were added Na₂CO₃ (333 mg, 3.14 mmol, 2.00 eq) and Pd(PPh₃)₄ (36.3 mg, 31.4 μmol, 0.0200 eq) at 25 °C. The resulting mixture was stirred at 80 °C for 2 h under N₂. LCMS showed (3-(pyrrolidin-1-yl)phenyl)boronic acid (036-1) was consumed completely and the desired mass was detected. The mixture was concentrated in vacuo. The mixture was purified by prep-TLC (SiO₂, PE:EtOAc=10:1) to give the title compound (036-2) (200 mg, 720 μmol, 45.9% yield) as a yellow solid. LCMS (ES+): m/z 278.0 [M+H]+.

Synthesis of cis-methyl 3-((5-fluoro-4-(3-(pyrrolidin-1-yl)phenyl)pyrimidin-2-yl)amino)cyclohexane-1-carboxylate (036-3). To a stirred solution of 2-chloro-5-fluoro-4-(3-(pyrrolidin-1-yl)phenyl)pyrimidine (036-2) (200 mg, 720.14 μmol, 1.00 eq) and cis-methyl 3-aminocyclohexane-1-carboxylate (113 mg, 720 μmol, 1.00 eq) in dioxane (4 mL) were added BINAP (44.8 mg, 72.0 μmol, 0.100 eq), Cs₂CO₃ (704 mg, 2.16 mmol, 3.00 eq) and Pd₂(dba)₃ (65.9 mg, 72.0 μmol, 0.100 eq) at 25 °C. The resulting mixture was stirred at 100 °C for 12 h under N₂. LCMS showed 2-chloro-5-fluoro-4-(3-(pyrrolidin-1-yl)phenyl)pyrimidine (036-2) was consumed completely. The mixture was concentrated in vacuo. The mixture was purified by prep-TLC (SiO₂, PE/EtOAc=2/1) to give the title compound (036-3) (160 mg, 402 μmol, 55.8% yield) as a yellow solid. LCMS (ES+): m/z 399.1 [M+H]+.

Synthesis of cis-3-((5-fluoro-4-(3-(pyrrolidin-1-yl)phenyl)pyrimidin-2-yl)amino)cyclohexane-1-carboxylic acid (036-4). To a stirred solution of cis-methyl 3-((5-fluoro-4-(3-(pyrrolidin-1-yl)phenyl)pyrimidin-2-yl)amino)cyclohexane-1-carboxylate (036-3) (160 mg, 402 μmol, 1.00 eq) in THF (2 mL) and H₂O (1 mL) was added LiOH·H₂O (33.7 mg, 803 μmol, 2.00 eq) at 25 °C. The resulting mixture was stirred at 25 °C for 12 h. LCMS showed cis-methyl 3-((5-fluoro-4-(3-(pyrrolidin-1-yl)phenyl)pyrimidin-2-yl)amino)cyclohexane-1-carboxylate (036-3) was consumed and the desired mass was detected. The reaction mixture was concentrated in vacuo to remove THF. HCl (1 M) was added to the mixture to adjust the pH to 2. Then the mixture was extracted with EtOAc (3 mL*3). The combined organic layers were dried over Na₂SO₄, filtered and the filtrate was concentrated in vacuo to give the title compound (036-4) (120 mg, crude) as a yellow solid. LCMS (ES+): m/z 385.1 [M+H]+.

Synthesis of cis-3-((5-fluoro-4-(3-(pyrrolidin-1-yl)phenyl)pyrimidin-2-yl)amino)cyclohexane-1-carboxamide (Compound 36). To a stirred solution of cis-3-((5-fluoro-4-(3-(pyrrolidin-1-yl)phenyl)pyrimidin-2-yl)amino)cyclohexane-1-carboxylic acid (036-4) (100 mg, 260 μmol, 1.00 eq) and DIEA (101 mg, 780 μmol, 136 μL, 3.00 eq) in DMF (0.5 mL) were added HATU (119 mg, 312 μmol, 1.20 eq), HOBt (17.6 mg, 130 μmol, 0.500 eq) and NH₄Cl (55.7 mg, 1.04 mmol, 4.00 eq) at 25 °C. The resulting mixture was stirred at 25 °C for 2 h. LCMS showed cis-3-((5-fluoro-4-(3-(pyrrolidin-1-yl)phenyl)pyrimidin-2-yl)amino)cyclohexane-1-carboxylic acid (036-4) was consumed and the desired mass was detected. The mixture was concentrated in vacuo. The mixture was purified by prep-HPLC (column: Phenomenex Luna C18 75*30 mm*3 μm; mobile phase: [water(FA)-ACN]; B %: 30%-70%, 8 min) to give the title compound (Compound 36) (8.80 mg, 20.9 μmol, 8.04% yield, 91.1% purity) as a yellow solid. LCMS (ES+): m/z 384.2 [M+H]+.

### Example 37. Synthesis of (S)-1-(3-((5-chloro-4-(3-(piperidin-1-yl)phenyl)pyrimidin-2-yl)amino)piperidin-1-yl)ethan-1-one (Compound 37)

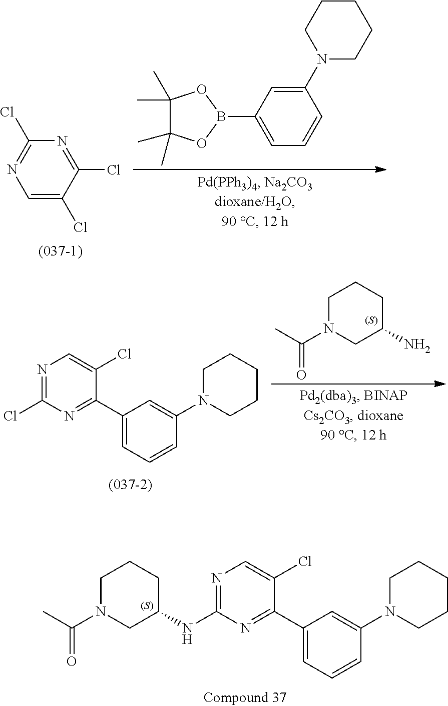

Synthesis of 2,5-dichloro-4-(3-(piperidin-1-yl)phenyl)pyrimidine (037-2). To a solution of 2,4,5-trichloropyrimidine (037-1) (229 mg, 1.25 mmol, 1.20 eq) and 1-(3-(4,4,5,5-tetramethyl-1,3,2-dioxaborolan-2-yl)phenyl)piperidine (300 mg, 1.04 mmol, 1.00 eq) in H₂O (1 mL) and dioxane (10 mL) were added Pd(PPh₃)₄ (60.1 mg, 52.0 μmol, 0.0500 eq) and Na₂CO₃ (220 mg, 2.08 mmol, 2.00 eq) at 25 °C under N₂ atmosphere. The mixture was stirred at 90 °C for 12 h. LCMS showed that the desired mass was detected. To the mixture was added H₂O (5 mL) and the mixture was extracted with EtOAc (5 mL*3). The combined organic layers were washed with saturated aqueous NaCl solution (5 mL*3), dried over Na₂SO₄, filtered and the filtrate was concentrated in vacuo. The mixture was purified by prep-TLC (SiO₂, PE/EtOAc=10/1) to give the title compound (037-2) (288 mg, 934 μmol, 44.9% yield) as a yellow oil liquid. LCMS (ES+): m/z 308.1 [M+H]+.

Synthesis of (S)-1-(3-((5-chloro-4-(3-(piperidin-1-yl)phenyl)pyrimidin-2-yl)amino)piperidin-1-yl)ethan-1-one (Compound 37). To a stirred solution of 2,5-dichloro-4-(3-(piperidin-1-yl)phenyl)pyrimidine (037-2) (130 mg, 442 μmol, 1.00 eq) and (S)-1-(3-aminopiperidin-1-yl)ethan-1-one (Compound C) (90.0 mg, 633 μmol, 1.50 eq) in dioxane (5 mL) were added BINAP (13.1 mg, 21.1 μmol, 0.0500 eq), Cs₂CO₃ (275 mg, 844 μmol, 2.00 eq) and Pd₂(dba)₃ (19.3 mg, 21.1 μmol, 0.0500 eq) at 25 °C. The resulting mixture was stirred at 90 °C for 12 h under N₂. LC-MS showed that 2,5-dichloro-4-(3-(piperidin-1-yl)phenyl)pyrimidine (037-2) was consumed and the desired mass was detected. To the mixture was added H₂O (3 mL) and the mixture was extracted with EtOAc (2 mL*3). The combined organic layers were washed with saturated aqueous NaCl solution (2 mL*3), dried over Na₂SO₄, filtered and the filtrate was concentrated in vacuo. The mixture was purified by acidic prep-HPLC (column: Phenomenex Luna C18 75*30 mm*3 μm; mobile phase: [water (FA)-ACN]; B %: 1%-50%, 8 min) to give the title compound (Compound 37) (25.5 mg, 60.5 μmol, 14.4% yield, 98.3% purity) as a white solid. LCMS (ES+): m/z 414.2 [M+H]+.

### Example 38. Synthesis of cis-3-((4-(3-cyclobutylphenyl)-5-fluoropyrimidin-2-yl)amino)cyclohexane-1-carboxamide (Compound 38)

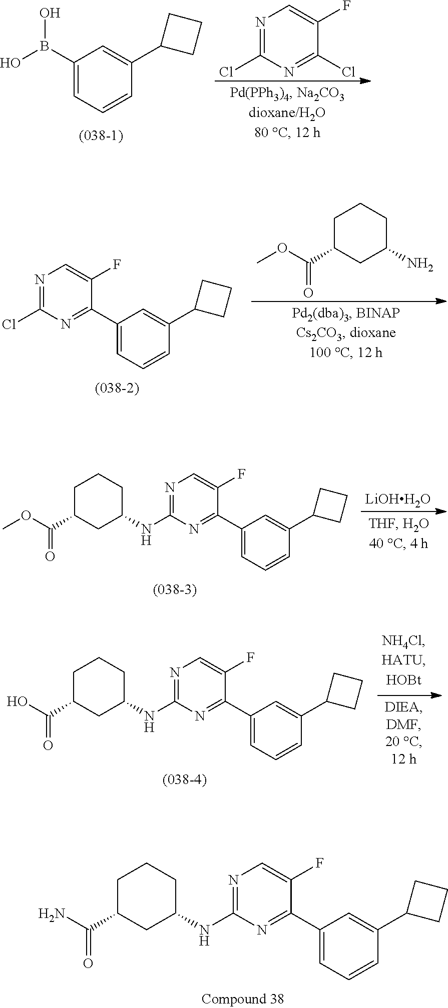

Synthesis of 2-chloro-4-(3-cyclobutylphenyl)-5-fluoropyrimidine (038-2). To a stirred solution of 2,4-dichloro-5-fluoropyrimidine (640 mg, 3.83 mmol, 1.50 eq) and (3-cyclobutylphenyl)boronic acid (038-1) (450 mg, 2.56 mmol, 1.00 eq) in dioxane (15 mL) and H₂O (1.5 mL) was added Na₂CO₃ (542 mg, 5.11 mmol, 2.00 eq) and Pd(PPh₃)₄ (59.1 mg, 51.1 μmol, 0.0200 eq) at 25 °C. The resulting mixture was stirred at 80 °C for 12 h under N₂. LCMS showed (3-cyclobutylphenyl)boronic acid (038-1) was consumed, desired target MS was detected. The mixture was concentrated in vacuo, then H₂O (15 mL) was added to the mixture and the mixture was extracted with EtOAc (15 mL*3), the combined organic layers were washed with saturated aqueous NaCl solution (15 mL*3), dried over Na₂SO₄, filtered and the filtrate was concentrated in vacuo to give the crude product. The mixture was purified by prep-TLC (SiO₂, PE/EtOAc=20/1) to give the title compound (038-2) (500 mg, 1.90 mmol, 74.4% yield) as a colorless oil. LCMS (ES+): m/z 263.0 [M+H]+.

Synthesis of cis-3-((4-(3-cyclobutylphenyl)-5-fluoropyrimidin-2-yl)amino)cyclohexane-1-carboxylate (038-3). To a stirred solution of 2-chloro-4-(3-cyclobutylphenyl)-5-fluoropyrimidine (038-2) (400 mg, 1.52 mmol, 1.00 eq) and cis-methyl 3-aminocyclohexane-1-carboxylate (295 mg, 1.52 mmol, 1.00 eq, HCl) in dioxane (12 mL) were added Cs₂CO₃ (1.49 g, 4.57 mmol, 3.00 eq), BINAP (94.8 mg, 152 μmol, 0.100 eq) and Pd₂(dba)₃ (139 mg, 152 μmol, 0.100 eq) at 25 °C. The resulting mixture was stirred at 100 °C for 12 h under N₂. LCMS showed 2-chloro-4-(3-cyclobutylphenyl)-5-fluoropyrimidine (038-2) was consumed, desired target MS was detected. The mixture was concentrated in vacuo, then H₂O (12 mL) was added to the mixture and the mixture was extracted with EtOAc (12 mL*3), the combined organic layers were washed with saturated aqueous NaCl solution (12 mL*3), dried over Na₂SO₄, filtered and the filtrate was concentrated in vacuo to give the crude product. The mixture was purified by prep-TLC (SiO₂, PE/EtOAc=3/1) to give the title compound (038-3) (230 mg, 600 μmol, 39.4% yield) as a yellow solid. LCMS (ES+): m/z 384.1 [M+H]+.

Synthesis of cis-3-((4-(3-cyclobutylphenyl)-5-fluoropyrimidin-2-yl)amino)cyclohexane-1-carboxylic acid (038-4). A solution of cis-3-((4-(3-cyclobutylphenyl)-5-fluoropyrimidin-2-yl)amino)cyclohexane-1-carboxylate (038-3) (230 mg, 600 μmol, 1.00 eq) in THF (3 mL) and H₂O (1.5 mL) was added LiOH·H₂O (50.3 mg, 1.20 mmol, 2.00 eq) at 25 °C, then the mixture was stirred at 40 °C for 2 h. LCMS showed cis-3-((4-(3-cyclobutylphenyl)-5-fluoropyrimidin-2-yl)amino)cyclohexane-1-carboxylate (038-3) was remained, desired target MS was detected, then the mixture was stirred at 40 °C for 2 h. LCMS showed starting material was consumed mostly, desired target MS was detected. The reaction mixture was concentrated in vacuo to remove THF. Then HCl (1 M) was added to the mixture to adjust the pH to 2. Then the mixture was extracted with EtOAc (3 mL*3). The combined organic layers were dried over Na₂SO₄, filtered and the filtrate was concentrated in vacuo to give the title compound (038-4) (220 mg, 596 μmol, 99.3% yield) as a yellow solid. LCMS (ES+): m/z: 370.1 [M+H]+.

Synthesis of cis-3-((4-(3-cyclobutylphenyl)-5-fluoropyrimidin-2-yl)amino)cyclohexane-1-carboxamide (Compound 38). To a solution of cis-3-((4-(3-cyclobutylphenyl)-5-fluoropyrimidin-2-yl)amino)cyclohexane-1-carboxylic acid (038-4) (110 mg, 298 μmol, 1.00 eq) and NH₄Cl (63.7 mg, 1.19 mmol, 4.00 eq) in DMF (1.5 mL) was added HATU (136 mg, 357 μmol, 1.20 eq), HOBt (20.1 mg, 149 μmol, 0.500 eq) and DIEA (192 mg, 1.49 mmol, 259 μL, 5.00 eq) at 25 °C, then the mixture was stirred at 25 °C for 12 h. LCMS showed cis-3-((4-(3-cyclobutylphenyl)-5-fluoropyrimidin-2-yl)amino)cyclohexane-1-carboxylic acid (038-4) was consumed, desired target MS was detected. The mixture was concentrated in vacuo to give the crude product. The residue was purified by prep-HPLC (column: Phenomenex Luna C18 200*40 mm*10 μm; mobile phase: [water (FA)-ACN]; B %: 40%-80%, 8 min) to give the title compound (Compound 38) (15.2 mg, 40.9 μmol, 13.7% yield, 99.2% purity) as a gray solid. LCMS (ES+): m/z 369.1 [M+H]+.

### Example 39. Synthesis of (S)-1-(3-((5-chloro-4-(3-cyclohexylphenyl)pyrimidin-2-yl)amino)piperidin-1l-yl)ethan-1-one (Compound 39)

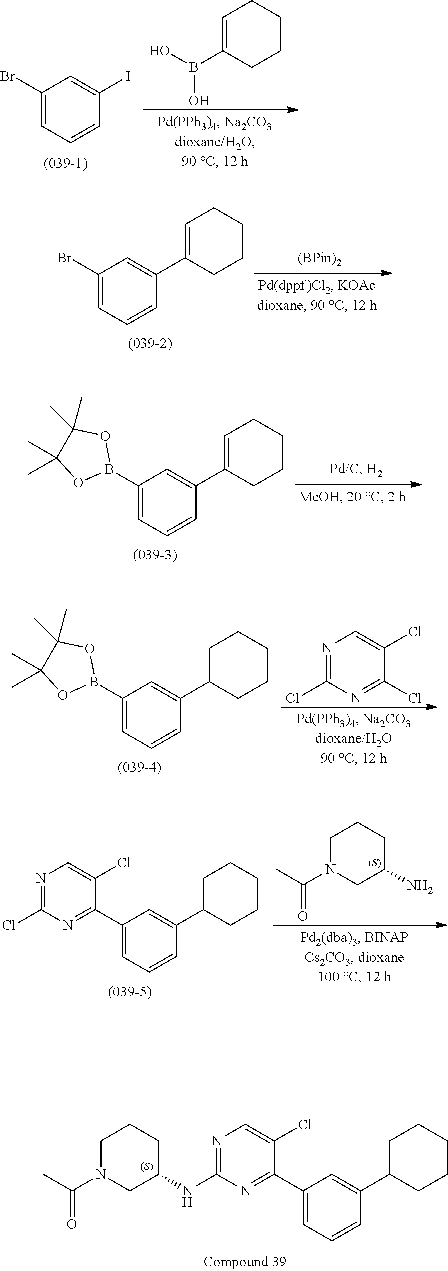

Synthesis of 3′-bromo-2,3,4,5-tetrahydro-1,1′-biphenyl (039-2). To a solution of 1-bromo-3-iodobenzene (039-1) (3.37 g, 11.9 mmol, 1.52 mL, 1.50 eq) in dioxane (40 mL) and H₂O (4 mL) were added Pd(PPh₃)₄ (183 mg, 159 μmol, 0.0200 eq), cyclohexen-1-ylboronic acid (1.00 g, 7.94 mmol, 1.00 eq) and Na₂CO₃ (1.68 g, 15.9 mmol, 2.00 eq) at 25 °C under N₂ atmosphere. The mixture was stirred at 90 °C for 12 h. TLC (SiO₂, Ethyl acetate/Petroleum ether=0/1) indicated 1-bromo-3-iodobenzene (039-1) was consumed completely and one new spot formed. Water (40 mL) was added to the mixture at 25 °C. Then the reaction mixture was extracted with EtOAc (30 mL*3). The organic layers were dried over anhydrous sodium sulfate, filtered and the filtrate was concentrated under reduced pressure to give a residue. The residue was purified by column chromatography (SiO₂, Petroleum ether/Ethyl acetate=1/0 to 99/1) to give the title compound (039-2) (1.80 g, 7.59 mmol, 95.6% yield) as a colorless oil.

Synthesis of 4,4,5,5-tetramethyl-2-(2′,3′,4′,5′-tetrahydro-[1,1′-biphenyl]-3-yl)-1,3,2-dioxaborolane (039-3). To a solution of 3′-bromo-2,3,4,5-tetrahydro-1,1′-biphenyl (039-2) (1.40 g, 5.90 mmol, 1.00 eq) in dioxane (30 mL) were added AcOK (1.16 g, 11.8 mmol, 2.00 eq), 4,4,5,5-tetramethyl-2-(4,4,5,5-tetramethyl-1,3,2-dioxaborolan-2-yl)-1,3,2-dioxaborolane (1.80 g, 7.08 mmol, 1.20 eq) and Pd(dppf)Cl₂ (216 mg, 295 μmol, 0.0500 eq) at 20 °C under N₂ atmosphere. The mixture was stirred at 90 °C for 12 h. LCMS showed the desired mass was detected. The reaction mixture was concentrated under reduced pressure to remove solvent. The residue was purified by column chromatography (SiO₂, Petroleum ether/Ethyl acetate=1/0 to 20/1) to give the title compound (039-3) (1.00 g, 3.52 mmol, 59.6% yield) as a colorless oil. LCMS (ES+): m/z 285.2 [M+H]+.

Synthesis of 2-(3-cyclohexylphenyl)-4,4,5,5-tetramethyl-1,3,2-dioxaborolane (039-4). To a solution of 4,4,5,5-tetramethyl-2-(2′,3′,4′,5′-tetrahydro-[1,1′-biphenyl]-3-yl)-1,3,2-dioxaborolane (039-3) (1.00 g, 3.52 mmol, 1.00 eq) in MeOH (20 mL) was added 10% Pd/C (1.00 g, 50% purity) at 20 °C under H₂ (15 psi) atmosphere. The mixture was stirred at 20 °C for 2 h. LCMS showed 4,4,5,5-tetramethyl-2-(2′,3′,4′,5′-tetrahydro-[1,1′-biphenyl]-3-yl)-1,3,2-dioxaborolane (039-3) was consumed completely and the desired mass was detected. The organic layers were filtered and the filtrate was concentrated under reduced pressure to give the title compound (039-4) (1.00 g, crude) as a colorless oil. LCMS (ES+): m/z 287.2 [M+H]+.

Synthesis of 2,5-dichloro-4-(3-cyclohexylphenyl)pyrimidine (039-5). To a solution of 2-(3-cyclohexylphenyl)-4,4,5,5-tetramethyl-1,3,2-dioxaborolane (039-4) (1.00 g, 3.49 mmol, 1.00 eq) in dioxane (20 mL) and H₂O (2 mL) were added Pd(PPh₃)₄ (80.8 mg, 69.9 μmol, 0.0200 eq), 2,4,5-trichloropyrimidine (1.28 g, 6.99 mmol, 2.00 eq) and Na₂CO₃ (741 mg, 6.99 mmol, 2.00 eq) at 20 °C under N₂ atmosphere. The mixture was stirred at 90 °C for 12 h. LCMS showed 2-(3-cyclohexylphenyl)-4,4,5,5-tetramethyl-1,3,2-dioxaborolane (039-4) was consumed completely and the desired mass was detected. Water (16 mL) was added to the mixture at 20 °C. Then the reaction mixture was extracted with EtOAc (15 mL*3). The organic layers were dried over anhydrous sodium sulfate, filtered and the filtrate was concentrated under reduced pressure to give a residue. The residue was purified by column chromatography (SiO₂, Petroleum ether/Ethyl acetate=1/0 to 10/1) to give the title compound (039-5) (700 mg, 2.28 mmol, 65.2% yield) as a colorless oil. LCMS (ES+): m/z 307.1 [M+H]+.

Synthesis of (S)-1-(3-((5-chloro-4-(3-cyclohexylphenyl)pyrimidin-2-yl)amino)piperidin-1-yl)ethan-1-one (Compound 39). To a solution of 1-[(3S)-3-amino-1-piperidyl]ethanone (104 mg, 732 μmol, 1.50 eq) in dioxane (5 mL) were added Pd₂(dba)₃ (22.4 mg, 24.4 μmol, 0.0500 eq), Cs₂CO₃ (318 mg, 977 μmol, 2.00 eq), 2,5-dichloro-4-(3-cyclohexylphenyl)pyrimidine (039-5) (150 mg, 488 μmol, 1.00 eq) and BINAP (15.2 mg, 24.4 μmol, 0.0500 eq) at 20 °C under N₂ atmosphere. The mixture was stirred at 100 °C for 12 h. LCMS showed 2,5-dichloro-4-(3-cyclohexylphenyl)pyrimidine (039-5) was consumed completely and the desired mass was detected. The reaction mixture was concentrated under reduced pressure to remove solvent. The residue was purified by prep-HPLC (column: Phenomenex Luna C18 75*30 mm*3 μm; mobile phase: [water(FA)-ACN]; B %: 40%-80%, 8 min) to give the title compound (Compound 39) (40.2 mg, 95.9 μmol, 19.7% yield, 98.5% purity) as a yellow solid. LCMS (ES+): m/z 413.1 [M+H]+.

### Example 40. Synthesis of 3-[[5-fluoro-4-[3-(2-oxo-1-pyridyl)phenyl]pyrimidin-2-yl]amino]cyclohexanecarboxamide (Compound 40)

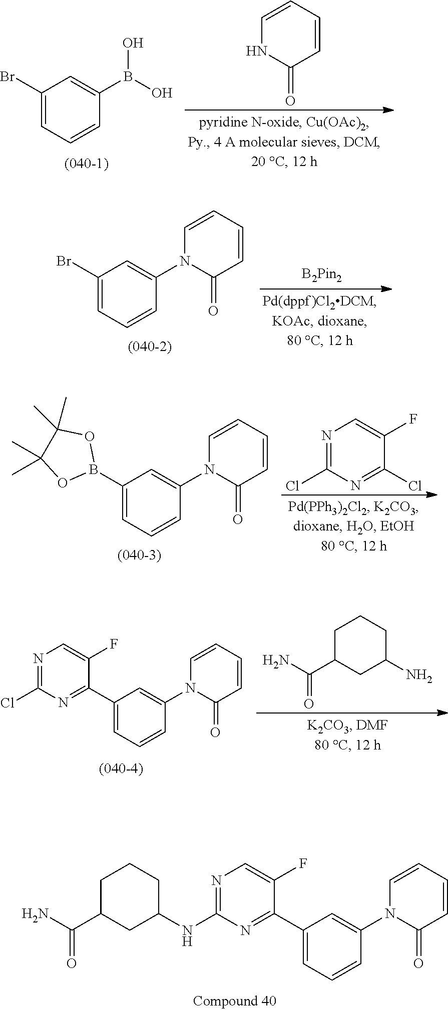

Synthesis of 1-(3-bromophenyl)pyridin-2-one (040-2). To a solution of 1H-pyridin-2-one (1.00 g, 10.5 mmol, 1.00 eq) and (3-bromophenyl)boronic acid (040-1) (4.22 g, 21.0 mmol, 2.00 eq) in DCM (25 mL) was added Cu(OAc)₂ (95.5 mg, 526 μmol, 0.0500 eq), 4 Å molecular sieve (200 mg) and Py (832 mg, 10.5 mmol, 849 μL, 1.00 eq) at 20 °C. The mixture was stirred at 20 °C for 12 h. LCMS showed that 1H-pyridin-2-one was consumed completely and one main peak with desired mass was detected. The mixture was filtered and the filtrate was concentrated under reduced pressure. The residue was purified by MPLC (SiO₂, Petroleum ether:Ethyl acetate=1:0 to 0:1) to give the title compound (040-2) (670 mg, 2.68 mmol, 25.5% yield) as a pink solid. LCMS (ES+): m/z 249.9 [M+H]+.

Synthesis of 1-[3-(4,4,5,5-tetramethyl-1,3,2-dioxaborolan-2-yl)phenyl]pyridin-2-one (040-3). To a solution of 1-(3-bromophenyl)pyridin-2-one (040-2) (650 mg, 2.60 mmol, 1.00 eq) and 4,4,5,5-tetramethyl-2-(4,4,5,5-tetramethyl-1,3,2-dioxaborolan-2-yl)-1,3,2-dioxaborolane (858 mg, 3.38 mmol, 1.30 eq) in dioxane (5 mL) was added KOAc (1.02 g, 10.4 mmol, 4.00 eq) and Pd(dppf)Cl₂·CH₂Cl₂ (212 mg, 260 μmol, 0.100 eq) at 20 °C. The mixture was stirred at 100 °C for 12 h under N₂. LCMS showed that 1-(3-bromophenyl)pyridin-2-one (040-2) was consumed completely and one main peak with desired mass was detected. The mixture was filtered and the filtrate was concentrated under reduced pressure. The residue was purified by MPLC (SiO₂, Petroleum ether:Ethyl acetate=1:0 to 0:1) to give the title compound (040-3) (510 mg, 1.72 mmol, 66.0% yield) as a yellow oil. LCMS (ES+): m/z 297.9 [M+H]+.

Synthesis of 1-[3-(2-chloro-5-fluoro-pyrimidin-4-yl)phenyl]pyridin-2-one (040-4). To a solution of 1-[3-(4,4,5,5-tetramethyl-1,3,2-dioxaborolan-2-yl)phenyl]pyridin-2-one (450 mg, 1.51 mmol, 1.00 eq) (040-3) and 2,4-dichloro-5-fluoro-pyrimidine (253 mg, 1.51 mmol, 1.00 eq) in dioxane (2 mL) were added Pd(PPh₃)₂Cl₂ (10.6 mg, 15.1 μmol, 0.0100 eq), H₂O (0.2 mL), EtOH (0.5 mL) and K₂CO₃ (1.05 g, 7.57 mmol, 5.00 eq) at 20 °C. The mixture was stirred at 80 °C for 12 h under N₂. LCMS showed 1-[3-(4,4,5,5-tetramethyl-1,3,2-dioxaborolan-2-yl)phenyl]pyridin-2-one (040-3) was remained and no desired mass was detected. The mixture was stirred at 80 °C for 12 h under N₂. LCMS showed 1-[3-(4,4,5,5-tetramethyl-1,3,2-dioxaborolan-2-yl)phenyl]pyridin-2-one (040-3) was consumed completely and desired mass was detected. The reaction mixture was concentrated under reduced pressure to remove solvent. The residue was purified by TLC (SiO₂, Petroleum ether/Ethyl acetate=0/1) to give the title compound (040-4) (284 mg, crude) as a white solid. LCMS (ES+): m/z 302.0 [M+H]+.

Synthesis of 3-[[5-fluoro-4-[3-(2-oxo-1-pyridyl)phenyl]pyrimidin-2-yl]amino]cyclohexanecarboxamide (Compound 40). To a solution of 1-[3-(2-chloro-5-fluoro-pyrimidin-4-yl)phenyl]pyridin-2-one (270 mg, 895 μmol, 1.00 eq) (040-4) and 3-aminocyclohexanecarboxamide (160 mg, 896 μmol, 1.00 eq, HCl) in DMF (5 mL) was added K₂CO₃ (371 mg, 2.68 mmol, 3.00 eq) at 20 °C. The mixture was stirred at 80 °C for 12 h. LCMS showed 1-[3-(2-chloro-5-fluoro-pyrimidin-4-yl)phenyl]pyridin-2-one (270 mg, 895 μmol, 1.00 eq) (040-4) was remained and desired mass was detected. The residue was diluted with H₂O (20 mL) and extracted with EtOAc (5 mL*3). The combined organic layers were washed with brine (20 mL*3), dried over Na₂SO₄, filtered and concentrated under reduced pressure to give a residue. The residue was purified by prep-HPLC (column: Phenomenex Luna C18 200*40 mm*10 μm; mobile phase: [water (FA)-ACN]; B %: 15%-45%, 8 min) to give the title compound (Compound 40) (12.5 mg, 30.2 μmol, 3.37% yield, 98.4% purity) as a white solid. LCMS (ES+): m/z 408.1 [M+H]+.

### Example 41. Synthesis of cis-3-((5-fluoro-4-(1-(4-fluorophenyl)-6-oxo-1,6-dihydropyridin-3-yl)pyrimidin-2-yl)amino)cyclohexane-1-carboxamide (Compound 41)

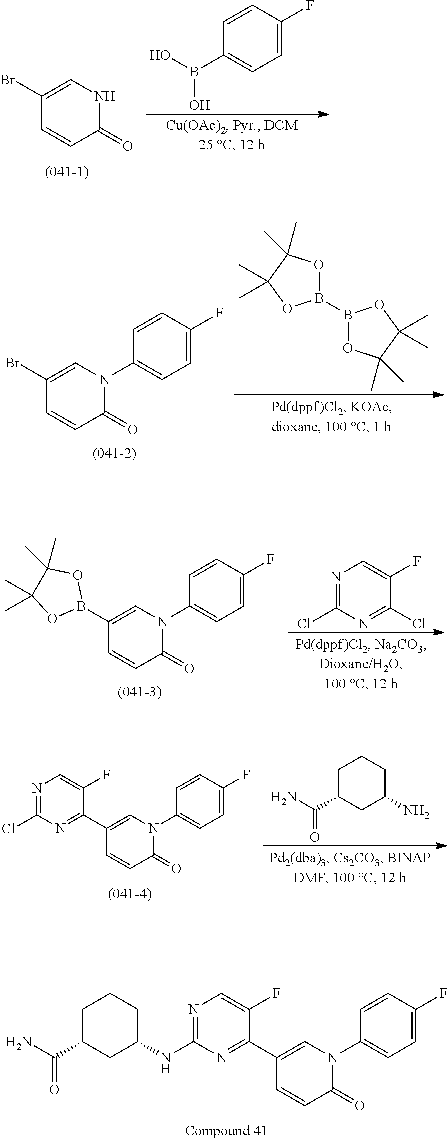

Synthesis of 5-bromo-1-(4-fluorophenyl)pyridin-2(1H)-one (041-2). To a solution of 5-bromopyridin-2(1H)-one (041-1) (4.00 g, 22.9 mmol, 1.00 eq) in DCM (100 mL) was added Cu(OAc)₂ (8.35 g, 45.9 mmol, 2.00 eq) and Py (9.09 g, 114 mmol, 9.28 mL, 5.00 eq), (4-fluorophenyl)boronic acid (9.65 g, 68.9 mmol, 3.00 eq) at 25 °C. The mixture was stirred at 25 °C for 12 h under air. LCMS showed the desired mass was detected. The reaction mixture was diluted with water 50 mL and extracted with EtOAc (50 mL*3). The combined organic layers were washed with brine (50 mL*3), dried over Na₂SO₄, filtered and concentrated under reduced pressure. The residue was purified by column chromatography (SiO₂, Petroleum ether/Ethyl acetate=1/0 to 1/1) to give the title compound (041-2) (5.00 g, 18.6 mmol, 81.1% yield) as a white solid. LCMS (ES+): m/z 268.0 [M+H]+.

Synthesis of 1-(4-fluorophenyl)-5-(4,4,5,5-tetramethyl-1,3,2-dioxaborolan-2-yl)pyridin-2(1H)-one (041-3). To a solution of 5-bromo-1-(4-fluorophenyl)pyridin-2(1H)-one (041-2) (2.00 g, 7.46 mmol, 1.00 eq) and 4,4,5,5-tetramethyl-2-(4,4,5,5-tetramethyl-1,3,2-dioxaborolan-2-yl)-1,3,2-dioxaborolane (1.89 g, 7.46 mmol, 1.00 eq) in dioxane (50 mL) was added Pd(dppf)Cl₂ (545 mg, 746 μmol, 0.100 eq) and KOAc (2.20 g, 22.3 mmol, 3.00 eq) at 25 °C. The mixture was stirred at 100 °C for 1 h under N₂. TLC (PE/EtOAc=1/1) indicated 5-bromo-1-(4-fluorophenyl)pyridin-2(1H)-one (041-2) was consumed completely and new spots formed. The reaction mixture was diluted with water 30 mL and extracted with EtOAc (30 mL*3). The combined organic layers dried over Na₂SO₄, filtered and concentrated under reduced pressure. The residue was purified by column chromatography (SiO₂, Petroleum ether/Ethyl acetate=1/0 to 1/1) to give the title compound (041-3) (2 g, 6.35 mmol, 85.1% yield) as a white solid. LCMS (ES+): m/z 316.0 [M+H]+.

Synthesis of 5-(2-chloro-5-fluoropyrimidin-4-yl)-1-(4-fluorophenyl)pyridin-2(1H)-one (041-4). To a solution of 1-(4-fluorophenyl)-5-(4,4,5,5-tetramethyl-1,3,2-dioxaborolan-2-yl)pyridin-2(1H)-one (041-3) (1.80 g, 5.71 mmol, 1.00 eq) and 2,4-dichloro-5-fluoro-pyrimidine (1.14 g, 6.85 mmol, 1.20 eq) in dioxane (50 mL) and H₂O (10 mL) was added Pd(dppf)Cl₂ (417 mg, 571 μmol, 0.100 eq) and Na₂CO₃ (1.82 g, 17.1 mmol, 3.00 eq) at 25 °C. The mixture was stirred at 100 °C for 2 h under N₂. LCMS showed 1-(4-fluorophenyl)-5-(4,4,5,5-tetramethyl-1,3,2-dioxaborolan-2-yl)pyridin-2(1H)-one (041-3) was consumed completely and the desired mass was detected. The reaction mixture was diluted with water 20 mL and extracted with EtOAc (20 mL*3). The combined organic layers were washed with brine (20 mL*3), dried over Na₂SO₄, filtered and concentrated under reduced pressure. The residue was purified by column chromatography (SiO₂, Petroleum ether/Ethyl acetate=1/0 to 1/1) to give the title compound (041-4) (1.00 g, crude) as a white solid. LCMS (ES+): m/z 320.0 [M+H]+.

Synthesis of cis-3-((5-fluoro-4-(1-(4-fluorophenyl)-6-oxo-1,6-dihydropyridin-3-yl)pyrimidin-2-yl)amino)cyclohexane-J-carboxamide (Compound 41). To a solution of 5-(2-chloro-5-fluoropyrimidin-4-yl)-1-(4-fluorophenyl)pyridin-2(1H)-one (041-4) (150 mg, 469 μmol, 1.00 eq) and cis-3-aminocyclohexane-1-carboxamide (80.1 mg, 563 μmol, 1.20 eq) in DMF (5 mL) was added Pd₂(dba)₃ (42.9 mg, 46.9 μmol, 0.100 eq), Cs₂CO₃ (458 mg, 1.41 mmol, 3.00 eq) and BINAP (29.2 mg, 46.9 μmol, 0.100 eq) at 25 °C. The mixture was stirred at 100 °C for 12 h under N₂. LCMS showed 5-(2-chloro-5-fluoropyrimidin-4-yl)-1-(4-fluorophenyl)pyridin-2(1H)-one (041-4) was consumed completely and the desired mass was detected. The reaction mixture was concentrated under reduced pressure. The residue was purified by prep-HPLC (column: Waters Xbridge BEH C18 100*30 mm*10 μm; mobile phase: [water (NH₄HCO₃)-ACN]; B %: 25%-55%, 8 min) to give the title compound (Compound 41) (16.7 mg, 39.2 μmol, 8.37% yield, 100% purity) as a white solid. LCMS (ES+): m/z 426.2 [M+H]+.

### Example 42. cis-3-((5-fluoro-4-(1-(4-fluorophenyl)-1H-pyrazol-3-yl)pyrimidin-2-yl)amino)cyclohexane-1-carboxamide (Compound 42)

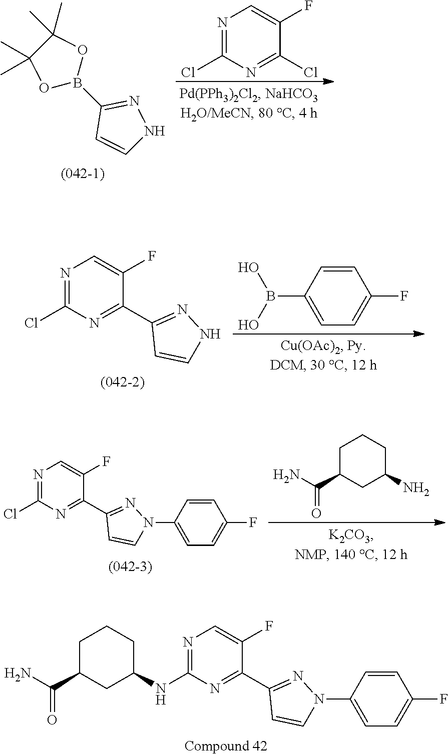

Synthesis of 2-chloro-5-fluoro-4-(1H-pyrazol-3-yl)pyrimidine (042-2). To a solution of 2,4-dichloro-5-fluoro-pyrimidine (2.15 g, 12.9 mmol, 1.00 eq) and 3-(4,4,5,5-tetramethyl-1,3,2-dioxaborolan-2-yl)-1H-pyrazole (042-1) (5.00 g, 25.8 mmol, 2.00 eq) in H₂O (10 mL) and MeCN (30 mL) were added Pd(PPh₃)₂Cl₂ (904 mg, 1.29 mmol, 0.100 eq) and NaHCO₃ (5.41 g, 64.4 mmol, 2.50 mL, 5.00 eq) at 25 °C. The mixture was stirred at 80 °C for 4 h under N₂ atmosphere. LCMS showed 3-(4,4,5,5-tetramethyl-1,3,2-dioxaborolan-2-yl)-1H-pyrazole (042-1) was consumed and the desired mass was detected. The reaction mixture was filtered and the filtrate was concentrated under reduced pressure to remove solvent. The reaction mixture was partitioned between H₂O 20 mL and EtOAc 20 mL. The organic phase was separated, washed with brine 10 mL, dried over Na₂SO₄, filtered and concentrated under reduced pressure to give the title compound (042-2) (1.80 g, crude) as a white solid. LCMS (ES+): m/z 198.9 [M+H]+.

Synthesis of 2-chloro-5-fluoro-4-(1-(4-fluorophenyl)-1H-pyrazol-3-yl)pyrimidine (042-3). To a solution of 2-chloro-5-fluoro-4-(1H-pyrazol-3-yl)pyrimidine (042-2) (1.00 g, 5.04 mmol, 1.00 eq) and (4-fluorophenyl)boronic acid (916 mg, 6.55 mmol, 1.30 eq) in DCM (15 mL) were added Cu(OAc)₂ (1.37 g, 7.55 mmol, 1.50 eq) and pyridine (1.19 g, 15.1 mmol, 1.22 mL, 3.00 eq) at 30 °C. The mixture was stirred at 30 °C for 12 h under N₂ atmosphere. LCMS showed 2-chloro-5-fluoro-4-(1H-pyrazol-3-yl)pyrimidine (042-2) was consumed and the desired mass was detected. The reaction mixture was filtered and the filtrate was concentrated under reduced pressure to remove solvent. The residue was purified by prep-HPLC (column: column: Phenomenex Luna C18 200*40 mm*10 μm; mobile phase: [water(FA)-ACN]; B %: 50%-80%, 8 min) to give the title compound (042-3) (500 mg, 1.62 mmol, 32.2% yield, 95.0% purity) as a brown solid. LCMS (ES+): m/z 293.0 [M+H]+.

Synthesis of cis-3-((5-fluoro-4-(1-(4-fluorophenyl)-1H-pyrazol-3-yl)pyrimidin-2-yl)amino)cyclohexane-1-carboxamide (Compound 42). To a solution of 2-chloro-5-fluoro-4-[1-(4-fluorophenyl)pyrazol-3-yl]pyrimidine (042-3) (115 mg, 393 μmol, 1.00 eq) and cis-3-aminocyclohexanecarboxamide (112 mg, 786 μmol, 2.00 eq) in NMP (3 mL) was added K₂CO₃ (163 mg, 1.18 mmol, 3.00 eq) at 25 °C. The mixture was stirred at 140 °C for 12 h. LCMS showed that 2-chloro-5-fluoro-4-[1-(4-fluorophenyl)pyrazol-3-yl]pyrimidine (042-3) was consumed and the desired mass was detected. The reaction mixture was filtered and the filtrate was concentrated under reduced pressure to remove solvent. The residue was purified by prep-HPLC (column: Phenomenex Luna C18 200*40 mm*10 μm; mobile phase: [water(FA)-ACN]; B %: 35%-65%, 8 min) to give the title compound (Compound 42) (12.9 mg, 32.2 μmol, 8.20% yield, 99.5% purity) as a creamy white solid. LCMS (ES+): m/z 399.1 [M+H]+.

### Example 43. cis-3-((5-fluoro-4-(1-(4-fluorophenyl)-1H-pyrazol-4-yl)pyrimidin-2-yl)amino)cyclohexane-1-carboxamide (Compound 43)

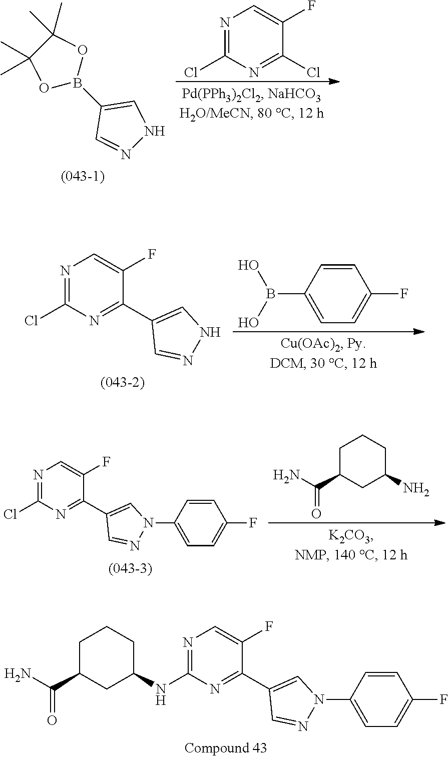

Synthesis of 2-chloro-5-fluoro-4-(1H-pyrazol-4-yl)pyrimidine (043-2). To a solution of 2,4-dichloro-5-fluoro-pyrimidine (2.15 g, 12.9 mmol, 1.00 eq) and 4-(4,4,5,5-tetramethyl-1,3,2-dioxaborolan-2-yl)-1H-pyrazole (043-1) (5.00 g, 25.8 mmol, 2.00 eq) in H₂O (10 mL) and MeCN (30 mL) was added Pd(PPh₃)₂Cl₂ (904 mg, 1.29 mmol, 0.100 eq) and NaHCO₃ (5.41 g, 64.4 mmol, 2.50 mL, 5.00 eq) at 25 °C. The mixture was stirred at 80 °C for 12 h under N₂ atmosphere. LCMS showed that 4-(4,4,5,5-tetramethyl-1,3,2-dioxaborolan-2-yl)-1H-pyrazole (043-1) was consumed and the desired mass was detected. The reaction mixture was filtered and the filtrate was concentrated under reduced pressure to remove solvent. The residue was partitioned between H₂O 20 mL and EtOAc 20 mL. The organic phase was separated, washed with brine 10 mL, dried over Na₂SO₄, filtered and concentrated under reduced pressure to give the title compound (043-2) (1.30 g, crude) as a white solid. LCMS (ES+): m/z 198.9 [M+H]+.

Synthesis of 2-chloro-5-fluoro-4-(1-(4-fluorophenyl)-1H-pyrazol-4-yl)pyrimidine (043-3). To a solution of 2-chloro-5-fluoro-4-(1H-pyrazol-4-yl) pyrimidine (043-2) (1.00 g, 5.04 mmol, 1.00 eq) and (4-fluorophenyl)boronic acid (916 mg, 6.55 mmol, 1.30 eq) in DCM (15 mL) were added Cu(OAc)₂ (1.37 g, 7.55 mmol, 1.50 eq) and Py (1.19 g, 15.1 mmol, 1.22 mL, 3.00 eq) at 30 °C. The mixture was stirred at 30 °C for 12 h under N₂ atmosphere. LCMS showed that 2-chloro-5-fluoro-4-(1H-pyrazol-4-yl) pyrimidine (043-2) was consumed and the desired mass was detected. The reaction mixture was filtered and the filtrate was concentrated. The residue was purified by prep-HPLC (column: column: Phenomenex Luna C18 200*40 mm*10 μm; mobile phase: [water(FA)-ACN]; B %: 50%-80%, 8 min) to give the title compound (043-3) (200 mg, 649 μmol, 12.9% yield, 95.0% purity) as a white solid. LCMS (ES+): m/z 293.0 [M+H]+.

Synthesis of cis-3-((5-fluoro-4-(1-(4-fluorophenyl)-1H-pyrazol-4-yl)pyrimidin-2-yl)amino)cyclohexane-1-carboxamide (Compound 43). To a solution of 2-chloro-5-fluoro-4-[1-(4-fluorophenyl)pyrazol-4-yl]pyrimidine (043-3) (130 mg, 444 μmol, 1.00 eq) and cis-3-aminocyclohexanecarboxamide (126 mg, 888 μmol, 2.00 eq) in NMP (3 mL) were added K₂CO₃ (184 mg, 1.33 mmol, 3.00 eq) at 25 °C. The mixture was stirred at 140 °C for 12 h. LCMS showed that 2-chloro-5-fluoro-4-[1-(4-fluorophenyl)pyrazol-4-yl]pyrimidine (043-3) was consumed and the desired mass was detected. The reaction mixture was filtered and the filtrate was concentrated under reduced pressure to remove solvent. The residue was purified by prep-HPLC (column: Phenomenex Luna C18 200*40 mm*10 μm; mobile phase: [water(FA)-ACN]; B %: 30%-60%, 8 min) to give the title compound (Compound 43) (35.5 mg, 88.2 μmol, 19.8% yield, 98.9% purity) as a white solid. LCMS (ES+): m/z 399.1 [M+H]+.

### Example 44. Synthesis of 3-(5-(1-(2-(4-((S)-3-((4-([1,1′-biphenyl]-3-yl)-5-chloropyrimidin-2-yl)amino)piperidine-1-carbonyl)piperidin-1-yl)ethyl)piperidin-4-yl)-3-methyl-2-oxo-2,3-dihydro-1H-benzo[d]imidazol-1-yl)piperidine-2,6-dione (Compound 44)

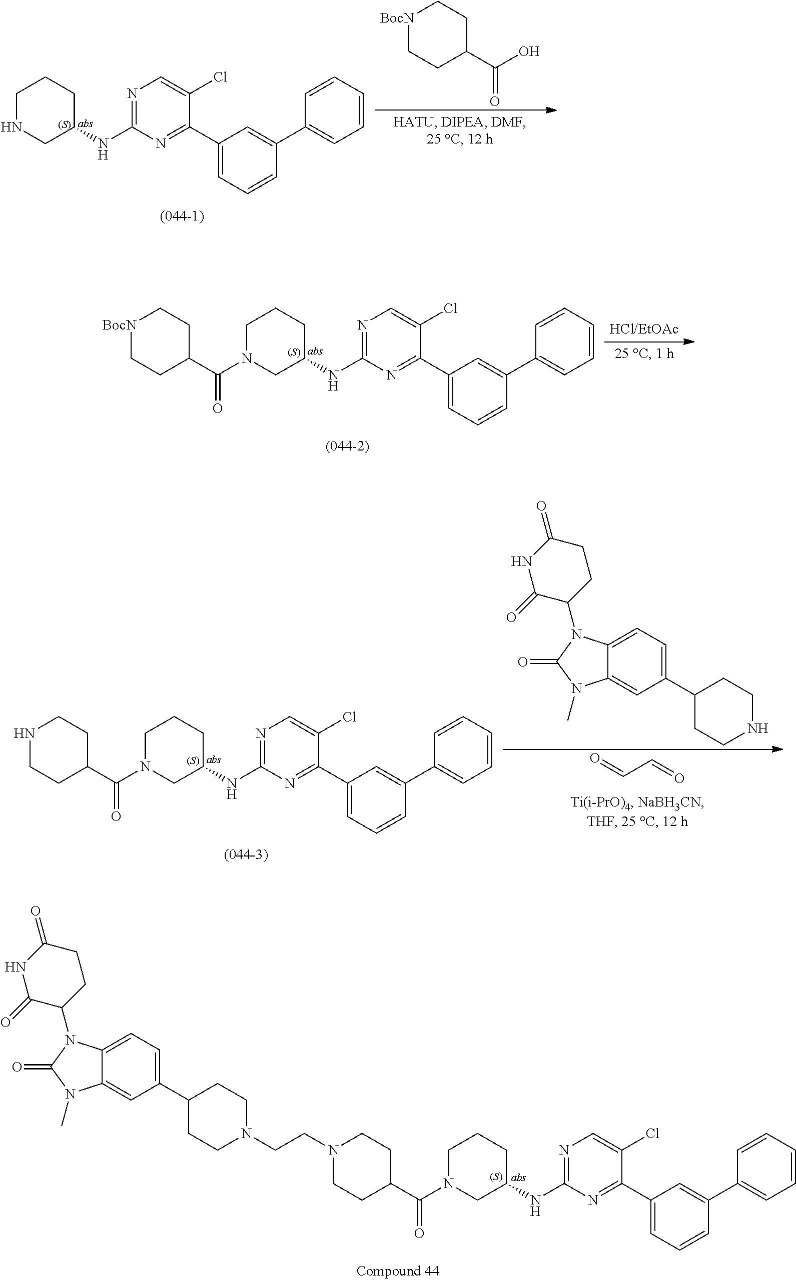

Synthesis of tert-butyl (S)-4-(3-((4-([1,1′-biphenyl]-3-yl)-5-chloropyrimidin-2-yl)amino)piperidine-1-carbonyl)piperidine-1-carboxylate (044-2). To a solution of 5-chloro-4-(3-phenylphenyl)-N-[(3S)-3-piperidyl]pyrimidin-2-amine (044-1) (4.69 g, 12.3 mmol, 1.50 eq) (prepared as described in Example 18) in DMF (10 mL) were added HATU (2.03 g, 5.34 mmol, 1.50 eq), 1-tert-butoxycarbonylpiperidine-4-carboxylic acid (2.26 g, 9.87 mmol, 1.20 eq) and DIEA (5.31 g, 41.1 mmol, 7.16 mL, 5.00 eq) at 25 °C. The mixture was stirred at 25 °C for 12 h. LCMS showed 5-chloro-4-(3-phenylphenyl)-N-[(3S)-3-piperidyl]pyrimidin-2-amine (044-1) was consumed completely and the desired mass was detected. The reaction mixture was diluted with H₂O (40 mL) and extracted with EtOAc (50 mL*3). The combined organic layers were dried over Na₂SO₄, filtered and the filtrate was concentrated under reduced pressure. The residue was purified by column chromatography (SiO₂, Petroleum ether:Ethyl acetate=10:1 to 2:1) to give the title compound (044-2) (4.00 g, 6.86 mmol, 83.5% yield, 98.9% purity) as a yellow oil. LCMS (ES+): m/z 576.2 [M+H]+.

Synthesis of (S)-(3-((4-([1,1′-biphenyl]-3-yl)-5-chloropyrimidin-2-yl)amino)piperidin-1-yl)(piperidin-4-yl)methanone (044-3). A solution of tert-butyl (S)-4-(3-((4-([1,1′-biphenyl]-3-yl)-5-chloropyrimidin-2-yl)amino)piperidine-1-carbonyl)piperidine-1-carboxylate (044-2) (4.00 g, 6.94 mmol, 1.00 eq) in HCl/EtOAc (4 M, 61.5 mL, 35.5 eq) was stirred at 25 °C for 1 h. LCMS showed the desired mass was detected. The reaction was concentrated under reduced pressure to give the title compound (4.50 g, crude) as a white solid. LCMS (ES+): m/z 476.2 [M+H]+.

Synthesis of 3-(5-(1-(2-(4-((S)-3-((4-([1,1′-biphenyl]-3-yl)-5-chloropyrimidin-2-yl)amino)piperidine-1-carbonyl)piperidin-1-yl)ethyl)piperidin-4-yl)-3-methyl-2-oxo-2,3-dihydro-1H-benzo[d]imidazol-1-yl)piperidine-2,6-dione (Compound 44). To a solution of (S)-(3-((4-([1,1′-biphenyl]-3-yl)-5-chloropyrimidin-2-yl)amino)piperidin-1-yl)(piperidin-4-yl)methanone (044-3) (200 mg, 420 μmol, 1.00 eq) and 3-[3-methyl-2-oxo-5-(4-piperidyl)benzimidazol-1-yl]piperidine-2,6-dione (143 mg, 420 μmol, 1.00 eq) in THF (5 mL) were added oxalaldehyde (24.3 mg, 421 μmol, 21.9 μL, 1.00 eq), Ti(i-PrO)₄ (597 mg, 2.10 mmol, 620 μL, 5.00 eq) and NaBH₃CN (132 mg, 2.10 mmol, 5.00 eq) at 25 °C. The mixture was stirred at 40 °C for 12 h. LCMS showed (S)-(3-((4-([1,1′-biphenyl]-3-yl)-5-chloropyrimidin-2-yl)amino)piperidin-1-yl)(piperidin-4-yl)methanone (044-3) was consumed completely and desired mass was detected. The reaction mixture was diluted with H₂O (20 mL) and extracted with EtOAc (20 mL*3). The combined organic layers were dried over Na₂SO₄, filtered and the filtrate was concentrated under reduced pressure. The residue was purified by prep-HPLC (column: Phenomenex Luna 80*30 mm*3 μm; mobile phase: [water(TFA)-ACN]; B %: 15%-45%, 8 min) to give the title compound (Compound 44) (34.5 mg, 40.8 μmol, 9.72% yield, 100% purity) as a white solid. LCMS (ES+): m/z 844.4 [M+H]+.

### Example 45. 3-[5-[1-[2-[4-[4-[[5-chloro-4-(3-phenylphenyl)pyrimidin-2-yl]amino]piperidine-1-carbonyl]-1-piperidyl]ethyl]-4-piperidyl]-3-methyl-2-oxo-benzimidazol-1-yl]piperidine-2,6-dione (Compound 45)

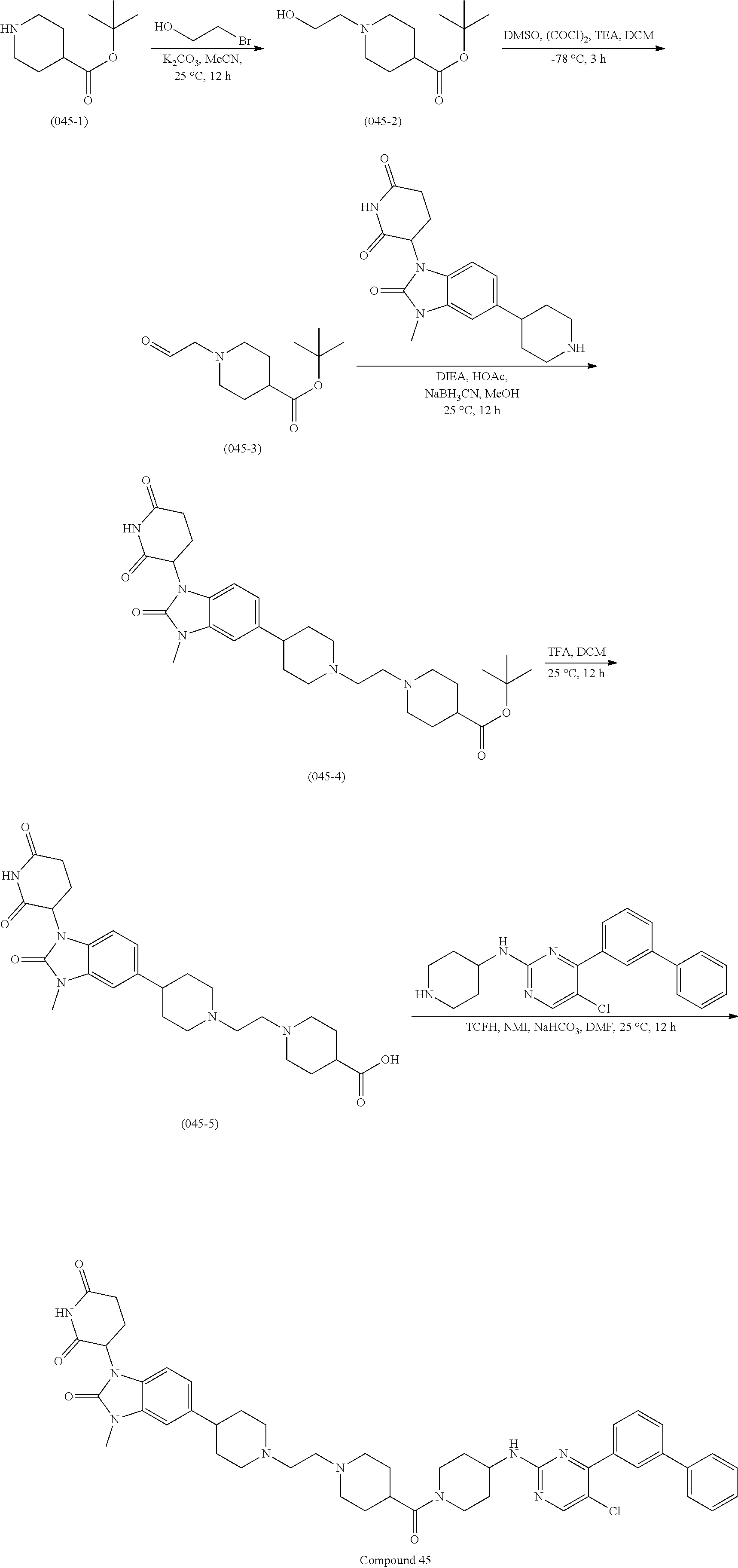

Synthesis of tert-butyl 1-(2-hydroxyethyl)piperidine-4-carboxylate (045-2). To a solution of tert-butyl piperidine-4-carboxylate (045-1) (8.00 g, 43.1 mmol, 1.00 eq) and 2-bromoethanol (6.48 g, 51.8 mmol, 3.68 mL, 1.20 eq) in MeCN (300 mL) was added K₂CO₃ (17.9 g, 129 mmol, 3.00 eq) at 25 °C. The mixture was stirred at 25 °C for 12 h. LCMS showed the desired mass was detected. The reaction mixture was diluted with H₂O (80 mL) and extracted with EtOAc (100 mL*3). The combined organic layers were washed with brine (50 mL), dried over Na₂SO₄, filtered and the filtrate was concentrated under reduced pressure. The residue was purified by column chromatography (SiO₂, DCM:MeOH=1:0 to 3:1) to give the title compound (045-2) (7.10 g, 21.0 mmol, 48.7% yield, 68.0% purity) as a yellow oil. LCMS (ES+): m/z 230.1 [M+H]+.

Synthesis of tert-butyl 1-(2-oxoethyl)piperidine-4-carboxylate (045-3). To a solution of oxalyl dichloride (1.16 g, 9.16 mmol, 801 μL, 2.10 eq) in DCM (3 mL) was added DMSO (1.16 g, 14.8 mmol, 1.16 mL, 3.40 eq) at −78 °C under N₂ atmosphere. The mixture was stirred at −78 °C for 1 h under N₂ atmosphere. Then tert-butyl 1-(2-hydroxyethyl)piperidine-4-carboxylate (045-2) (1.00 g, 4.36 mmol, 1.00 eq) was added. The mixture was stirred at −78 °C for 1 h, then TEA (2.91 g, 28.7 mmol, 4.01 mL, 6.60 eq) was added. The mixture was stirred at −78 °C for 1 h. LCMS showed the desired mass was detected. The reaction mixture was diluted with H₂O (15 mL) and extracted with DCM mL (20 mL*3). The combined organic layers were washed with brine (10 mL), dried over Na₂SO₄, filtered and the filtrate was concentrated under reduced pressure to give the title compound (045-3) (1.30 g, crude) as a yellow oil. LCMS (ES+): m/z 228.1 [M+H]+.

Synthesis of tert-butyl 1-[2-[4-[1-(2,6-dioxo-3-piperidyl)-3-methyl-2-oxo-benzimidazol-5-yl]-1-piperidyl]ethyl]piperidine-4-carboxylate (045-4). To a solution of 3-[3-methyl-2-oxo-5-(4-piperidyl)benzimidazol-1-yl]piperidine-2,6-dione (979 mg, 2.86 mmol, 0.500 eq) in MeOH (10 mL) was added DIEA (739 mg, 5.72 mmol, 996 μL, 1.00 eq) at 25 °C until pH~8. Then AcOH (343 mg, 5.72 mmol, 327 μL, 1.00 eq) and tert-butyl 1-(2-oxoethyl)piperidine-4-carboxylate (045-3) (1.30 g, 5.72 mmol, 1.00 eq) were added the mixture. Then the mixture was stirred at 25 °C, for 0.5 h, then sodium cyanoborohydride (1.08 g, 17.1 mmol, 3.00 eq) was added to the mixture at 25 °C. The mixture was stirred at 25 °C for 11.5 h. LCMS showed the desired mass was detected. The reaction mixture was diluted with H₂O (10 mL) and extracted with EtOAc (10 mL*3). The combined organic layers were washed with brine (10 mL), dried over Na₂SO₄, filtered and the filtrate was concentrated under reduced pressure. The residue was purified by column chromatography (SiO₂, DCM:MeOH=1:0 to 10:1) to give the title compound (045-4) (590 mg, 532 μmol, 9.32% yield, 50.0% purity) as a colorless gum. LCMS (ES+): m/z 554.3 [M+H]+.

Synthesis of 1-[2-[4-[1-(2,6-dioxo-3-piperidyl)-3-methyl-2-oxo-benzimidazol-5-yl]-1-piperidyl]ethyl]piperidine-4-carboxylic acid (045-5). To a solution of tert-butyl 1-[2-[4-[1-(2,6-dioxo-3-piperidyl)-3-methyl-2-oxo-benzimidazol-5-yl]-1-piperidyl]ethyl]piperidine-4-carboxylate (045-4) (570 mg, 1.03 mmol, 1.00 eq) in DCM (5 mL) was added TFA (3.08 g, 27.0 mmol, 2 mL, 26.2 eq) at 25 °C. The mixture was stirred at 25 °C for 12 h. LCMS showed the desired mass was detected. The reaction concentrated under reduced pressure to give the title compound (045-5) (500 mg, crude) as a yellow oil. LCMS (ES+): m/z 498.2 [M+H]+.

Synthesis of 3-[5-[1-[2-[4-[4-[[5-chloro-4-(3-phenylphenyl)pyrimidin-2-yl]amino]piperidine-1-carbonyl]-1-piperidyl]ethyl]-4-piperidyl]-3-methyl-2-oxo-benzimidazol-1-yl]piperidine-2,6-dione (Compound 45). To a solution of 1-[2-[4-[1-(2,6-dioxo-3-piperidyl)-3-methyl-2-oxo-benzimidazol-5-yl]-1-piperidyl]ethyl]piperidine-4-carboxylic acid (045-5) (160 mg, 321 μmol, 1.00 eq) in DMF (2 mL) was added NaHCO₃ (27.0 mg, 321 μmol, 12.5 μL, 1.00 eq) until pH~7. To a solution of 5-chloro-4-(3-phenylphenyl)-N-(4-piperidyl)pyrimidin-2-amine (Compound B) (58.6 mg, 160 μmol, 0.500 eq) in DMF (2 mL) was added NaHCO₃ (27.0 mg, 321 μmol, 12.5 μL, 1.00 eq) until pH~7. This two mixture was mixture. Then NMI (79.2 mg, 964 μmol, 76.9 μL, 3.00 eq) and TCFH (99.2 mg, 353 μmol, 1.10 eq) was added to it at 25 °C. The mixture was stirred at 25 °C for 12 h. LCMS showed the desired mass was detected. The reaction mixture was diluted with H₂O (10 mL) and extracted with EtOAc (15 mL*3). The combined organic layers were washed with brine (10 mL), dried over Na₂SO₄, filtered and the filtrate was concentrated under reduced pressure. The residue was purified by prep-HPLC (column: Phenomenex luna C18 80*40 mm*3 μm; mobile phase: [water (FA)-ACN]; B %: 25%-60%, 8 min) to give the title compound (Compound 45) (8.00 mg, 9.13 μmol, 2.84% yield, 96.4% purity) as a white solid. LCMS (ES+): m/z 844.4 [M+H]+.

### Example 46. Synthesis of 3-(5-(1-(2-(4-(4-((4-([1,1′-biphenyl]-3-yl)-5-chloropyrimidin-2-yl)amino)piperidine-1-carbonyl)piperidin-1-yl)acetyl)piperidin-4-yl)-3-methyl-2-oxo-2,3-dihydro-1H-benzo[d]imidazol-1-yl)piperidine-2,6-dione (Compound 46)

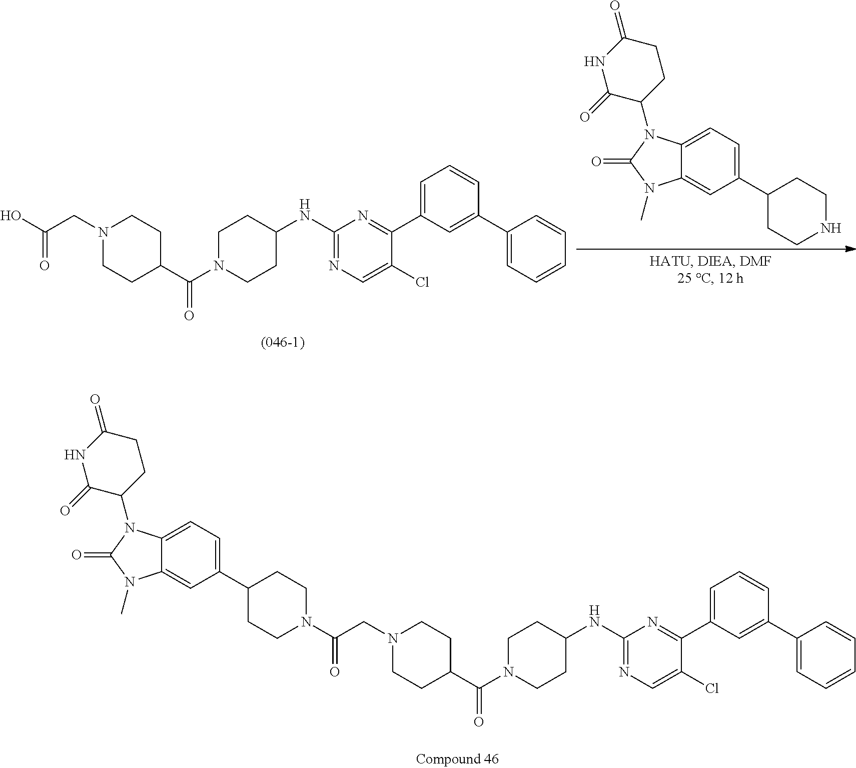 [SMILES: O=C(O)CN1CCC(C(=O)N2CCC(Nc3ncc(Cl)c(-c4cccc(-c5ccccc5)c4)n3)CC2)CC1 ; Cn1c(=O)n(C2CCC(=O)NC2=O)c2ccc(C3CCNCC3)cc21 ; Cn1c(=O)n(C2CCC(=O)NC2=O)c2ccc(C3CCN(C(=O)CN4CCC(C(=O)N5CCC(Nc6ncc(Cl)c(-c7cccc(-c8ccccc8)c7)n6)CC5)CC4)CC3)cc21]

Synthesis of 3-(5-(1-(2-(4-(4-((4-([1,1′-biphenyl]-3-yl)-5-chloropyrimidin-2-yl)amino)piperidine-1-carbonyl)piperidin-1-yl)acetyl)piperidin-4-yl)-3-methyl-2-oxo-2,3-dihydro-1H-benzo[d]imidazol-1-yl)piperidine-2,6-dione (Compound 46). To a solution of 2-[4-[4-[[5-chloro-4-(3-phenylphenyl)pyrimidin-2-yl]amino]piperidine-1-carbonyl]-1-piperidyl]acetic acid (200 mg, 374.5 μmol, 1.00 eq) (046-1) (prepared as described in Example 56) in DMF (2 mL) were added HATU (214 mg, 562 μmol, 1.50 eq), 3-[3-methyl-2-oxo-5-(4-piperidyl)benzimidazol-1-yl]piperidine-2,6-dione (154 mg, 449 μmol, 1.20 eq) (prepared by methods known in the art) and DIEA (145 mg, 1.12 mmol, 196 μL, 3.00 eq) at 25 °C. The mixture was stirred at 25 °C for 12 h. LCMS showed 2-[4-[4-[[5-chloro-4-(3-phenylphenyl)pyrimidin-2-yl]amino]piperidine-1-carbonyl]-1-piperidyl]acetic acid (046-1) was consumed completely and the desired mass was detected. The reaction mixture was diluted with H₂O 20 mL and extracted with EtOAc (30 mL*3). The combined organic layers were dried over Na₂SO₄, filtered and the filtrate was concentrated under reduced pressure. The residue was purified by prep-HPLC (column: C18-6 100*30 mm*5 μm; mobile phase: [water(FA)-ACN]; B %: 25%-65%, 8 min) to give the title compound (Compound 46) (14.4 mg, 16.4 μmol, 4.38% yield, 98.7% purity) as a light pink solid. LCMS (ES+): m/z 858.3 [M+H]+.

### Example 47. Synthesis of 3-(5-(1-((1-(2-(4-((4-([1,1′-biphenyl]-3-yl)-5-chloropyrimidin-2-yl)amino)piperidin-1-yl)-2-oxoethyl)piperidin-4-yl)methyl)piperidin-4-yl)-3-methyl-2-oxo-2,3-dihydro-1H-benzo[d]imidazol-1-yl)piperidine-2,6-dione (Compound 47)

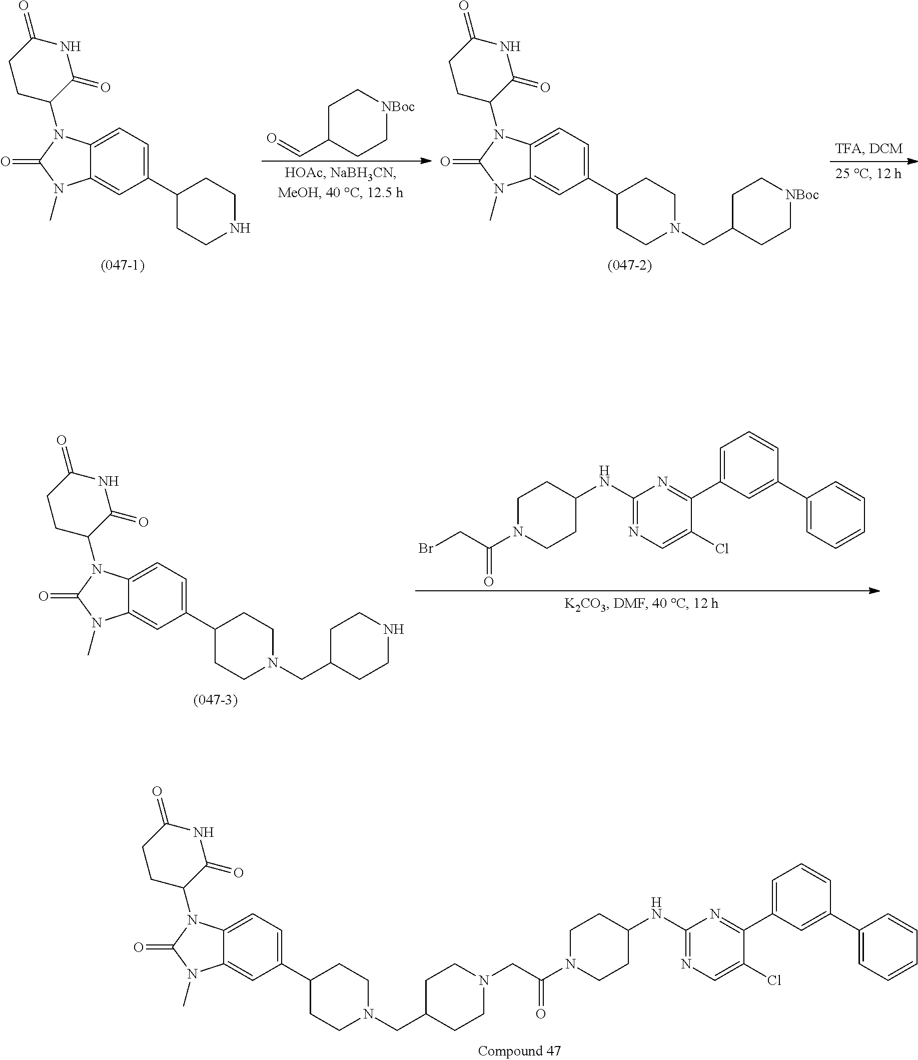 [SMILES: Cn1c(=O)n(C2CCC(=O)NC2=O)c2ccc(C3CCNCC3)cc21 ; CC(C)(C)OC(=O)N1CCC(C=O)CC1 ; Cn1c(=O)n(C2CCC(=O)NC2=O)c2ccc(C3CCN(CC4CCN(C(=O)OC(C)(C)C)CC4)CC3)cc21 ; Cn1c(=O)n(C2CCC(=O)NC2=O)c2ccc(C3CCN(CC4CCNCC4)CC3)cc21 ; O=C(CBr)N1CCC(Nc2ncc(Cl)c(-c3cccc(-c4ccccc4)c3)n2)CC1 ; Cn1c(=O)n(C2CCC(=O)NC2=O)c2ccc(C3CCN(CC4CCN(CC(=O)N5CCC(Nc6ncc(Cl)c(-c7cccc(-c8ccccc8)c7)n6)CC5)CC4)CC3)cc21]

Synthesis of tert-butyl 4-((4-(1-(2,6-dioxopiperidin-3-yl)-3-methyl-2-oxo-2,3-dihydro-1H-benzo[d]imidazol-5-yl)piperidin-1-yl)methyl)piperidine-1-carboxylate (047-2). To a solution of 3-(3-methyl-2-oxo-5-(piperidin-4-yl)-2,3-dihydro-1H-benzo[d]imidazol-1-yl)piperidine-2,6-dione (047-1) (2.00 g, 5.84 mmol, 1.00 eq) (prepared by methods known in the art) in MeOH (10 mL) were added HOAc (1.75 g, 29.2 mmol, 1.67 mL, 5.00 eq), tert-butyl 4-formylpiperidine-1-carboxylate (1.62 g, 7.59 mmol, 1.30 eq) at 25 °C, the mixture was stirred at 25 °C for 0.5 h, then NaBH₃CN (734 mg, 11.7 mmol, 2.00 eq) was added to the mixture at 25 °C. The mixture was stirred at 40 °C for 12 h. LCMS showed the desired mass was detected. Water (50 mL) was added to the solution, the mixture was extracted with ethyl acetate (50 mL*3), the combined organic layers were dried over Na₂SO₄, and the filtrate was concentrated under reduced pressure. The residue was purified by column chromatography (SiO₂, Dichloromethane:Methanol=10:1 to 5:1) to give the title compound (047-2) (1.34 g, 2.33 mmol, 40.0% yield, 94.0% purity) as a light yellow oil. LCMS (ES+): m/z 540.3 [M+H]+.

Synthesis of 3-(3-methyl-2-oxo-5-(1-(piperidin-4-ylmethyl)piperidin-4-yl)-2,3-dihydro-1H-benzo[d]imidazol-1-yl)piperidine-2,6-dione (047-3). To a solution of tert-butyl 4-((4-(1-(2,6-dioxopiperidin-3-yl)-3-methyl-2-oxo-2,3-dihydro-1H-benzo[d]imidazol-5-yl)piperidin-1-yl)methyl)piperidine-1-carboxylate (047-2) (1.20 g, 2.22 mmol, 1.00 eq) in DCM (9 mL) was added TFA (2.54 g, 22.2 mmol, 1.65 mL, 10.0 eq) at 25 °C. The mixture was stirred at 25 °C for 12 h. LCMS showed the desired mass was detected. The reaction was concentrated under reduced pressure to give the title compound (047-3) (2.50 g, crude) as a white solid. LCMS (ES+): m/z 440.3 [M+H]+.

Synthesis of 3-(5-(1-((1-(2-(4-((4-([1,1′-biphenyl]-3-yl)-5-chloropyrimidin-2-yl)amino)piperidin-1-yl)-2-oxoethyl)piperidin-4-yl)methyl)piperidin-4-yl)-3-methyl-2-oxo-2,3-dihydro-1H-benzo[d]imidazol-1-yl)piperidine-2,6-dione (Compound 47). To a solution of 3-(3-methyl-2-oxo-5-(1-(piperidin-4-ylmethyl)piperidin-4-yl)-2,3-dihydro-1H-benzo[d]imidazol-1-yl)piperidine-2,6-dione (047-3) (100 mg, 228 μmol, 1.00 eq) in DMF (2 mL) were added K₂CO₃ (94.3 mg, 683 μmol, 3.00 eq) and 2-bromo-1-[4-[[5-chloro-4-(3-phenylphenyl)pyrimidin-2-yl]amino]-1-piperidyl]ethanone (144 mg, 296 μmol, 1.30 eq) (prepared as described in Example 83) at 25 °C. The mixture was stirred at 40 °C for 12 h. LCMS showed 3-(3-methyl-2-oxo-5-(1-(piperidin-4-ylmethyl)piperidin-4-yl)-2,3-dihydro-1H-benzo[d]imidazol-1-yl)piperidine-2,6-dione (047-3) was consumed completely and the desired mass was detected. The reaction was concentrated under reduced pressure. The residue was purified by prep-HPLC (column: Phenomenex Luna C18 75*30 mm*3 μm; mobile phase: [water(FA)-ACN]; B %: 20%-55%, 8 min) to give the title compound (Compound 47). (11.4 mg, 12.6 μmol, 5.53% yield, 93.2% purity) as a white solid. LCMS (ES+): m/z 844.4 [M+H]+.

### Example 48. Synthesis of 3-((4-(1′-(3-(4-((4-([1,1′-biphenyl]-3-yl)-5-chloropyrimidin-2-yl)amino)piperidin-1-yl)-3-oxopropyl)-[1,4′-bipiperidin]-4-yl)phenyl)amino)piperidine-2,6-dione (Compound 48)

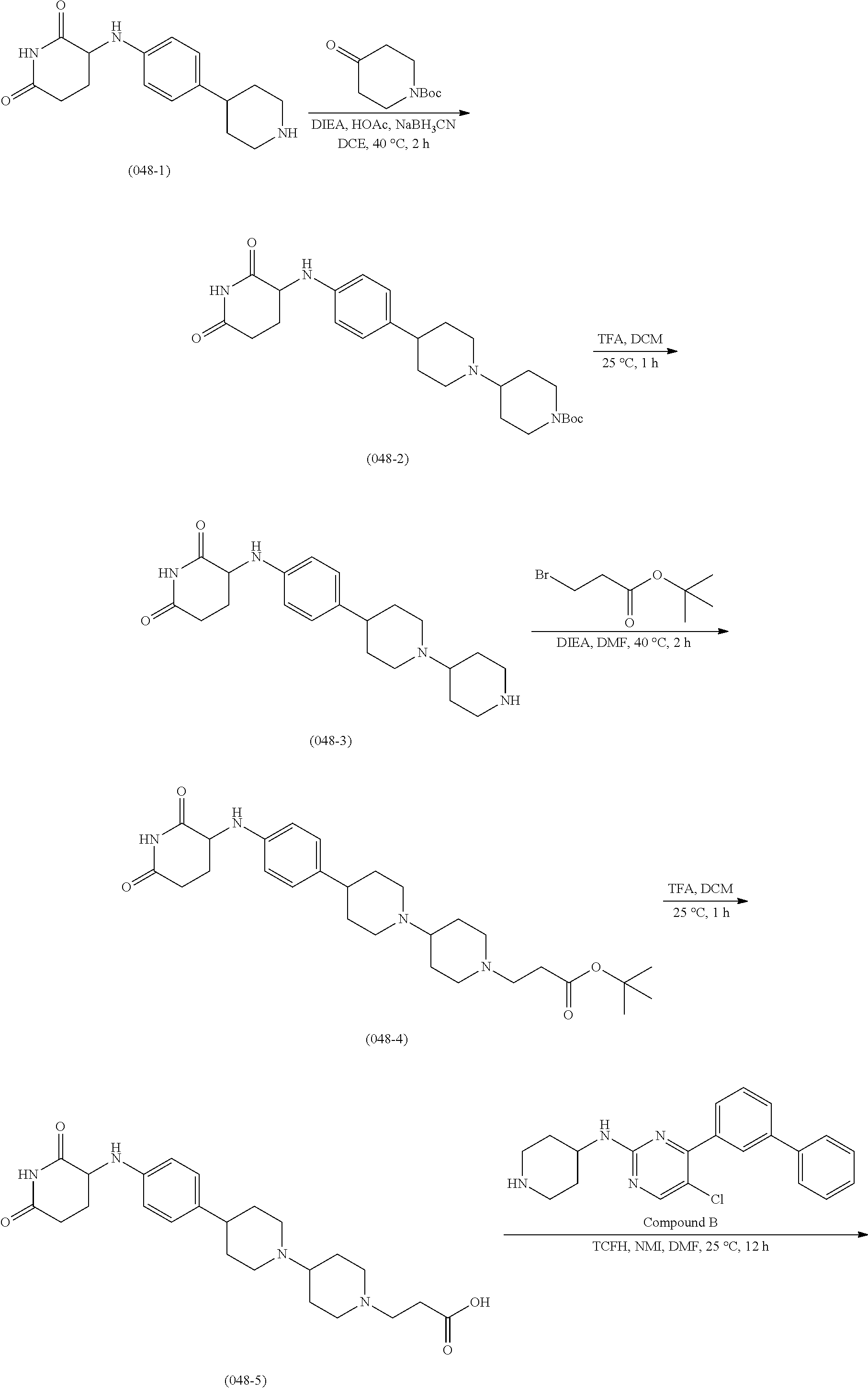

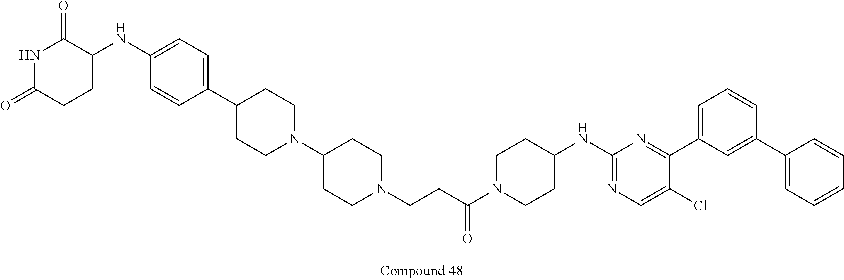 [SMILES: O=C1CCC(Nc2ccc(C3CCN(C4CCN(CCC(=O)N5CCC(Nc6ncc(Cl)c(-c7cccc(-c8ccccc8)c7)n6)CC5)CC4)CC3)cc2)C(=O)N1]

Synthesis of tert-butyl 4-(4-((2,6-dioxopiperidin-3-yl)amino)phenyl)-[1,4′-bipiperidine]-1′-carboxylate (048-2). To a solution of 3-[4-(4-piperidyl)anilino]piperidine-2,6-dione (048-1) (2.00 g, 6.96 mmol, 1.00 eq) in DCE (20 mL) was added was added DIBA until pH~7, then HOAc (2.09 g, 34.8 mmol, 1.99 mL, 5.00 eq) and tert-butyl 4-oxopiperidine-1-carboxylate (9.71 g, 48.7 mmol, 7.00 eq) were added to the mixture at 25 °C the mixture was stirred at 25 °C for 0.5 h, then NaBH₃CN (2.19 g, 34.8 mmol, 5.00 eq) was added to the mixture, The mixture was stirred at 40 °C for 1.5 h. LCMS showed 3-[4-(4-piperidyl)anilino]piperidine-2,6-dione (048-1) was consumed completely and the desired mass was detected. Water (50 mL) was added to the solution, the mixture was extracted with ethyl acetate (50 mL*3), the combined organic layers were dried over Na₂SO₄, and the filtrate was concentrated under reduced pressure to give a residue. The residue was purified by column chromatography (SiO₂, Dichloromethane:Methanol=10:1 to 5:1) to give the title compound (048-2) (3.50 g, 6.54 mmol, 94.0% yield, 88.0% purity) as a white solid. LCMS (ES+): m/z 471.2 [M+H]+.

Synthesis of 3-[4-[1-(4-piperidyl)-4-piperidyl]anilino]piperidine-2,6-dione (048-3). To a solution of tert-butyl 4-(4-((2,6-dioxopiperidin-3-yl)amino)phenyl)-[1,4′-bipiperidine]-1′-carboxylate (048-2) (3.00 g, 6.37 mmol, 1.00 eq) in DCM (6 mL) was added TFA (7.27 g, 63.7 mmol, 4.72 mL, 10.0 eq) at 25 °C. The mixture was stirred at 25 °C for 1 h. LCMS showed tert-butyl 4-(4-((2,6-dioxopiperidin-3-yl)amino)phenyl)-[1,4′-bipiperidine]-1′-carboxylate (048-2) was consumed completely and the desired mass was detected. The reaction was concentrated under reduced pressure to give the title compound (048-3) (6.80 g, crude) as a white solid. LCMS (ES+): m/z 371.2 [M+H]+.

Synthesis of tert-butyl 3-[4-[4-[4-[(2,6-dioxo-3-piperidyl)amino]phenyl]-1-piperidyl]-1-piperidyl]propanoate (048-4). To a solution of 3-[4-[1-(4-piperidyl)-4-piperidyl]anilino]piperidine-2,6-dione (048-3) (2.00 g, 5.40 mmol, 1.00 eq) in DMF (2 mL) were added DIEA (3.49 g, 26.9 mmol, 4.70 mL, 5.00 eq) and tert-butyl 3-bromopropanoate (1.13 g, 5.40 mmol, 902 μL, 1.00 eq) at 25 °C. The mixture was stirred at 40 °C for 2 h. LCMS showed 3-[4-[1-(4-piperidyl)-4-piperidyl]anilino]piperidine-2,6-dione (048-3) was consumed completely and the desired mass was detected. Water (50 mL) was added to the solution, the mixture was extracted with ethyl acetate (50 mL*3), the combined organic layers were dried over Na₂SO₄, and the filtrate was concentrated under reduced pressure. The residue was purified by column chromatography (SiO₂, Dichloromethane:Methanol=10:1 to 5:1) to give the title compound (048-4) (1.18 g, 2.08 mmol, 38.5% yield, 88.0% purity) as a white solid. LCMS (ES+): m/z 499.3 [M+H]+.

Synthesis of 3-(4-(4-((2,6-dioxopiperidin-3-yl)amino)phenyl)-[1,4′-bipiperidin]-1′-yl)propanoic acid (048-5). To a solution of tert-butyl 3-[4-[4-[4-[(2,6-dioxo-3-piperidyl)amino]phenyl]-1-piperidyl]-1-piperidyl]propanoate (048-4) (1.10 g, 2.21 mmol, 1.00 eq) in DCM (10 mL) was added TFA (2.52 g, 22.0 mmol, 1.63 mL, 10.0 eq) at 25 °C. The mixture was stirred at 25 °C for 1 h. LCMS showed tert-butyl 3-[4-[4-[4-[(2,6-dioxo-3-piperidyl)amino]phenyl]-1-piperidyl]-1-piperidyl]propanoate (048-4) was consumed completely and the desired mass was detected. The reaction concentrated under reduced pressure to give the title compound (048-5) (2.2 g, crude) as a brown oil. LCMS (ES+): m/z 443.3 [M+H]+.

Synthesis of 3-((4-(1′-(3-(4-((4-([1,1′-biphenyl]-3-yl)-5-chloropyrimidin-2-yl)amino)piperidin-1-yl)-3-oxopropyl)-[1,4′-bipiperidin]-4-yl)phenyl)amino)piperidine-2,6-dione (Compound 48). To a solution of 3-[4-[4-[4-[(2,6-dioxo-3-piperidyl)amino]phenyl]-1-piperidyl]-1-piperidyl]propanoic acid (048-5) (100 mg, 225 μmol, 1.00 eq), 5-chloro-4-(3-phenylphenyl)-N-(4-piperidyl)pyrimidin-2-amine (Compound B) (82.4 mg, 225 μmol, 1.00 eq) in DMF (2 mL) were added TCFH (69.7 mg, 248 μmol, 1.10 eq) and NMI (57.5 mg, 700 μmol, 55.8 μL, 3.10 eq) at 25 °C. The mixture was stirred at 25 °C for 12 h. LCMS showed (048-5) was consumed completely and desired mass was detected. The reaction mixture was filtered to give the filtrate. The residue was purified by prep-HPLC (column: Phenomenex Luna 80*30 mm*3 μm; mobile phase: [water(TFA)-ACN]; B %: 15%-45%, 8 min) to give the title compound (Compound 48) (5.00 mg, 6.18 μmol, 2.73% yield, 97.5% purity) as a green solid. LCMS (ES+): m/z 789.3 [M+H]+.

### Example 49. Synthesis of cis-3-(5-(1-(2-(4-(3-((4-([1,1′-biphenyl]-3-yl)-5-chloropyrimidin-2-yl)amino)cyclohexane-1-carbonyl)piperazin-1-yl)ethyl)piperidin-4-yl)-3-methyl-2-oxo-2,3-dihydro-1H-benzo[d]imidazol-1-yl)piperidine-2,6-dione (Compound 49)

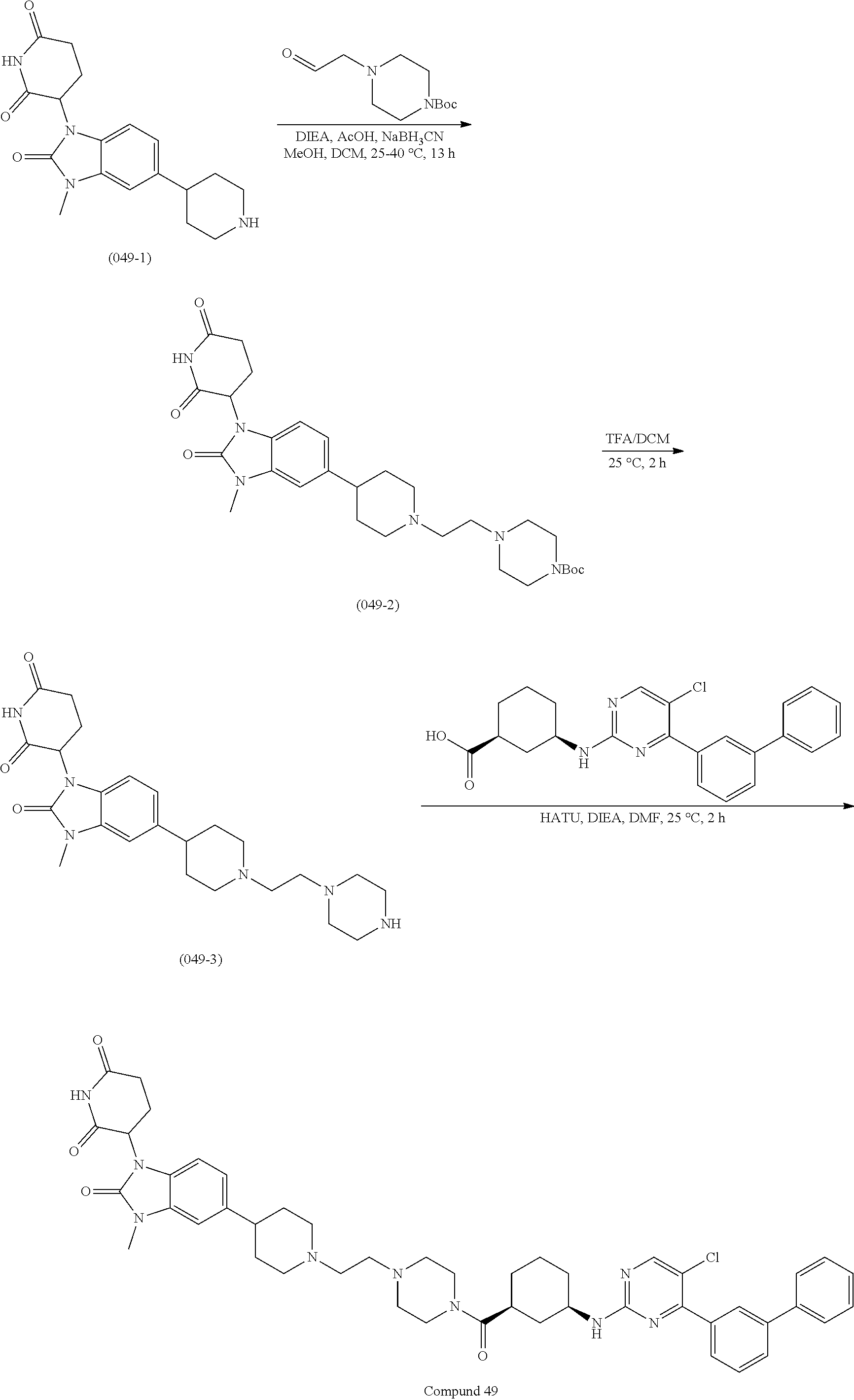 [SMILES: Cn1c(=O)n(C2CCC(=O)NC2=O)c2ccc(C3CCNCC3)cc21 ; CC(C)(C)OC(=O)N1CCN(CC=O)CC1 ; Cn1c(=O)n(C2CCC(=O)NC2=O)c2ccc(C3CCN(CCN4CCN(C(=O)OC(C)(C)C)CC4)CC3)cc21 ; Cn1c(=O)n(C2CCC(=O)NC2=O)c2ccc(C3CCN(CCN4CCNCC4)CC3)cc21 ; O=C(O)[C@H]1CCC[C@@H](Nc2ncc(Cl)c(-c3cccc(-c4ccccc4)c3)n2)C1 ; Cn1c(=O)n(C2CCC(=O)NC2=O)c2ccc(C3CCN(CCN4CCN(C(=O)[C@H]5CCC[C@@H](Nc6ncc(Cl)c(-c7cccc(-c8ccccc8)c7)n6)C5)CC4)CC3)cc21]

Synthesis of tert-butyl 4-(2-(4-(1-(2,6-dioxopiperidin-3-yl)-3-methyl-2-oxo-2,3-dihydro-1H-benzo[d]imidazol-5-yl)piperidin-1-yl)ethyl)piperazine-1-carboxylate (049-2). To a stirred solution of 3-(3-methyl-2-oxo-5-(piperidin-4-yl)-2,3-dihydro-1H-benzo[d]imidazol-1-yl)piperidine-2,6-dione (049-1) (480 mg, 1.05 mmol, 1.00 eq, TFA) in DCM (2 mL) and MeOH (4 mL) was added DIEA (136 mg, 1.05 mmol, 183 μL, 1.00 eq) at 25 °C and the mixture was stirred at 25 °C for 0.5 h. Then to the mixture was added tert-butyl 4-(2-oxoethyl)piperazine-1-carboxylate (480 mg, 2.10 mmol, 2.00 eq) (prepared as described in Example 55) and AcOH (6.32 mg, 105 μmol, 6.01 μL, 0.100 eq) at 25 °C. The mixture was stirred at 40 °C for 0.5 h. Then to the mixture was added NaBH₃CN (264 mg, 4.21 mmol, 4.00 eq) at 25 °C. The resulting mixture was stirred at 40 °C for 12 h. LCMS showed that 3-(3-methyl-2-oxo-5-(piperidin-4-yl)-2,3-dihydro-1H-benzo[d]imidazol-1-yl)piperidine-2,6-dione (049-1) was consumed and the desired target MS was detected. The mixture was diluted with H₂O (20 mL) and then extracted with EtOAc (15 mL*3). The combined organic layers were dried over Na₂SO₄, filtered and the filtrate was concentrated under reduced pressure. The mixture was purified by MPLC (SiO₂, DCM/MeOH=1/0 to 1/1) to give the title compound (049-2) (350 mg, 631 μmol, 64.0% yield) as a yellow oil. LCMS (ESI+): m/z 555.2 [M+H]+.

Synthesis of 3-(3-methyl-2-oxo-5-(1-(2-(piperazin-1-yl)ethyl)piperidin-4-yl)-2,3-dihydro-1H-benzo[d]imidazol-1-yl)piperidine-2,6-dione (049-3). A solution of tert-butyl 4-(2-(4-(1-(2,6-dioxopiperidin-3-yl)-3-methyl-2-oxo-2,3-dihydro-1H-benzo[d]imidazol-5-yl)piperidin-1-yl)ethyl)piperazine-1-carboxylate (049-2) (350 mg, 631 μmol, 1.00 eq) in DCM (10 mL) and TFA (2 mL) was stirred at 25 °C for 2 h. LCMS showed that tert-butyl 4-(2-(4-(1-(2,6-dioxopiperidin-3-yl)-3-methyl-2-oxo-2,3-dihydro-1H-benzo[d]imidazol-5-yl)piperidin-1-yl)ethyl)piperazine-1-carboxylate (049-2) was consumed and the desired target MS was detected. The mixture was concentrated under reduced pressure to give the title compound (049-3) (220 mg, crude, TFA) as a yellow oil. LCMS (ESI+): m/z 455.3 [M+H]+.

Synthesis of cis-3-(5-(1-(2-(4-(3-((4-([1,1′-biphenyl]-3-yl)-5-chloropyrimidin-2-yl)amino)cyclohexane-1-carbonyl)piperazin-1-yl)ethyl)piperidin-4-yl)-3-methyl-2-oxo-2,3-dihydro-1H-benzo[d]imidazol-1-yl)piperidine-2,6-dione (Compound 49). To a solution of cis-3-((4-([1,1′-biphenyl]-3-yl)-5-chloropyrimidin-2-yl)amino)cyclohexane-1-carboxylic acid (125 mg, 307 μmol, 1.09 eq) (prepared by methods analogous to those shown in Example 22) in DMF (5 mL) were added (3-methyl-2-oxo-5-(1-(2-(piperazin-1-yl)ethyl)piperidin-4-yl)-2,3-dihydro-1H-benzo[d]imidazol-1-yl)piperidine-2,6-dione (049-3) (160 mg, 281 μmol, 1.00 eq, TFA), HATU (128 mg, 338 μmol, 1.20 eq) and DIEA (182 mg, 1.41 mmol, 245 μL, 5.00 eq) at 25 °C, the mixture was stirred at 25 °C for 2 h. LCMS showed that 3-(3-methyl-2-oxo-5-(1-(2-(piperazin-1-yl)ethyl)piperidin-4-yl)-2,3-dihydro-1H-benzo[d]imidazol-1-yl)piperidine-2,6-dione (049-3) was consumed and the desired target MS was detected. H₂O (10 mL) was added to the solution, the mixture was extracted with EtOAc (5 mL*3), the combined organic layers were dried over Na₂SO₄, filtered and the filtrate was concentrated under reduced pressure to give the crude product. The residue was purified by prep-HPLC (column: Waters Xbridge BEH C18 100*30 mm*10 μm; mobile phase: [water(NH₄HCO₃)-ACN]; B %: 50%-80%, 8 min) to give the title compound (Compound 49) (37.8 mg, 44.4 μmol, 12.0% yield, 98.1% purity) as a white solid. LCMS (ESI+): m/z 844.3 [M+H]+.

### Example 50. Synthesis of 3-[5-[1-[[1-[2-[4-[[5-chloro-4-(3-phenylphenyl)pyrimidin-2-yl]amino]-1-piperidyl]-2-oxo-ethyl]-4-piperidyl]methyl]-4-piperidyl]-1-oxo-indan-2-yl]piperidine-2,6-dione (Compound 50)

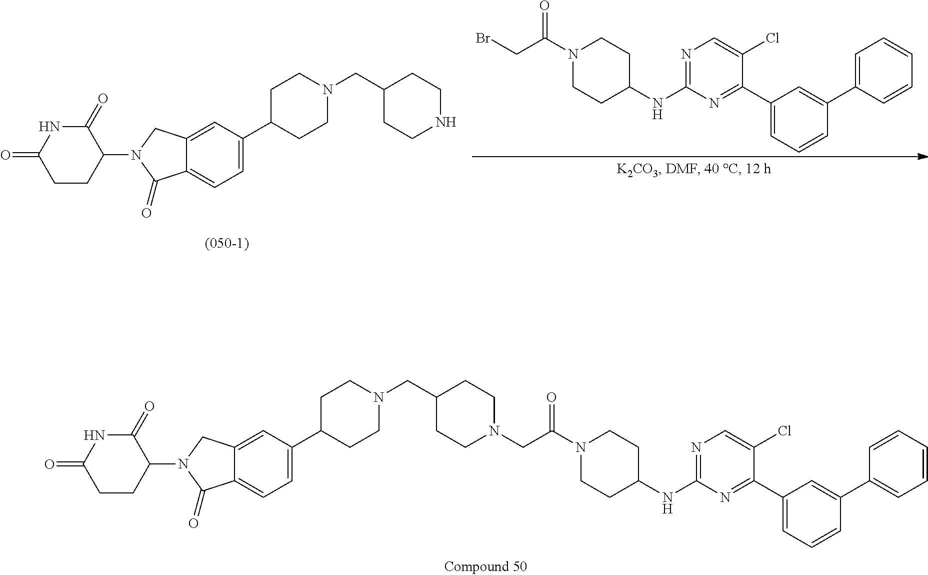 [SMILES: O=C1CCC(N2Cc3cc(C4CCN(CC5CCNCC5)CC4)ccc3C2=O)C(=O)N1 ; O=C(CBr)N1CCC(Nc2ncc(Cl)c(-c3cccc(-c4ccccc4)c3)n2)CC1 ; O=C1CCC(N2Cc3cc(C4CCN(CC5CCN(CC(=O)N6CCC(Nc7ncc(Cl)c(-c8cccc(-c9ccccc9)c8)n7)CC6)CC5)CC4)ccc3C2=O)C(=O)N1]

Synthesis of 3-[5-[1-[[1-[2-[4-[[5-chloro-4-(3-phenylphenyl)pyrimidin-2-yl]amino]-1-piperidyl]-2-oxo-ethyl]-4-piperidyl]methyl]-4-piperidyl]-1-oxo-indan-2-yl]piperidine-2,6-dione (Compound 50). To a solution of 3-[1-oxo-5-[1-(4-piperidylmethyl)-4-piperidyl]isoindolin-2-yl]piperidine-2,6-dione (050-1) (100 mg, 235 μmol, 1.00 eq) (prepared as described in Example 62) and 2-bromo-1-[4-[[5-chloro-4-(3-phenylphenyl) pyrimidin-2-yl]amino]-1-piperidyl]ethanone (137 mg, 282 μmol, 1.20 eq) (prepared as described in Example 83) in DMF (3 mL) was added K₂CO₃ (97.6 mg, 706 μmol, 3.00 eq) at 25 °C. The mixture was stirred at 40 °C for 12 h. LCMS showed the desired mass was detected. The reaction mixture was diluted with H₂O 10 mL and extracted with EtOAc (10 mL*3). The combined organic layers were washed with brine (10 mL), dried over Na₂SO₄, filtered and the filtrate was concentrated under reduced pressure. The residue was purified by prep-HPLC (column: Phenomenex Luna C18 75*30 mm*3 μm; mobile phase: [water (FA)-ACN]; B %: 20%-55%, 8 min) to give the title compound (Compound 50) (40.6 mg, 47.5 μmol, 20.1% yield, 99.1% purity, FA) as a white solid. LCMS (ES+): m/z 829.4 [M+H]+.

### Example 51. Synthesis of cis-3-(5-(1-(2-(4-(3-((4-([1,1′-biphenyl]-3-yl)-5-chloropyrimidin-2-yl)amino)cyclohexane-1-carbonyl)piperazin-1-yl)-2-oxoethyl)piperidin-4-yl)-3-methyl-2-oxo-2,3-dihydro-1H-benzo[d]imidazol-1-yl)piperidine-2,6-dione (Compound 51)

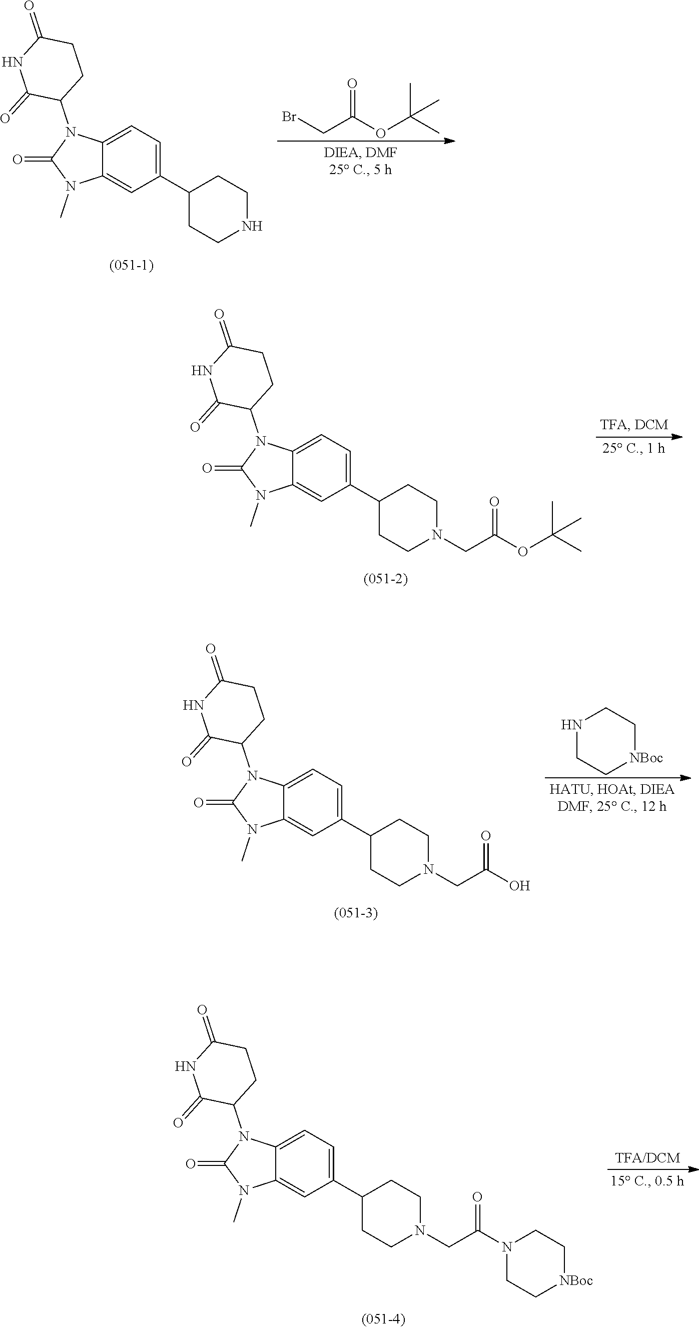

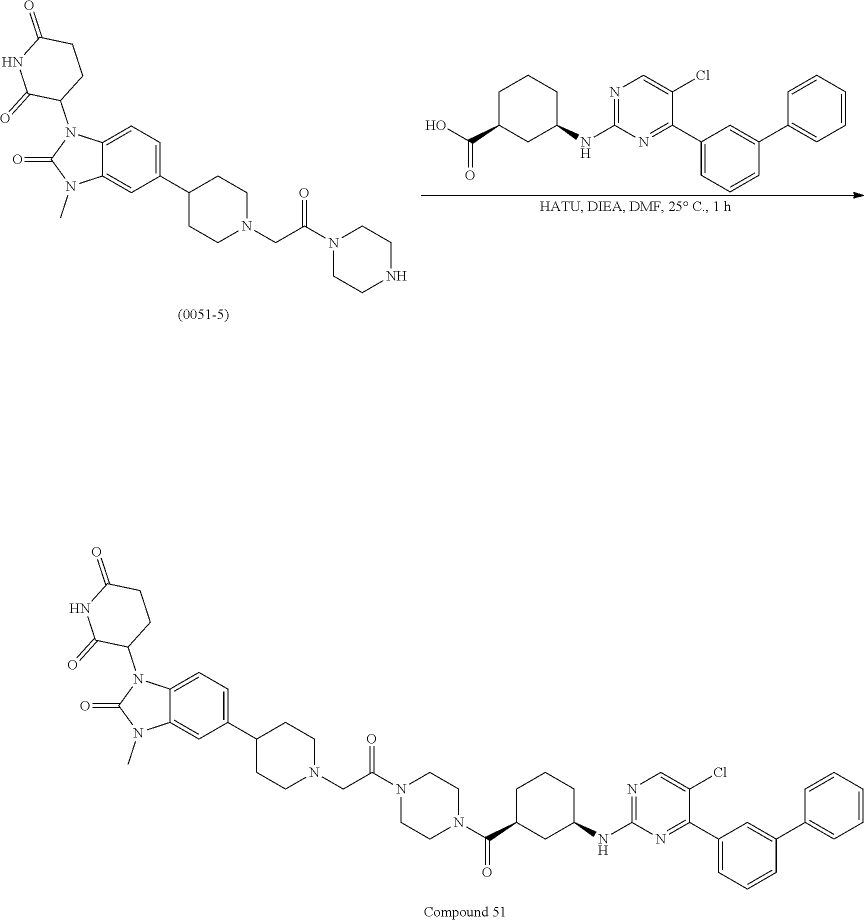 [SMILES: Cn1c(=O)n(C2CCC(=O)NC2=O)c2ccc(C3CCN(CC(=O)N4CCNCC4)CC3)cc21 ; O=C(O)[C@H]1CCC[C@@H](Nc2ncc(Cl)c(-c3cccc(-c4ccccc4)c3)n2)C1 ; Cn1c(=O)n(C2CCC(=O)NC2=O)c2ccc(C3CCN(CC(=O)N4CCN(C(=O)[C@H]5CCC[C@@H](Nc6ncc(Cl)c(-c7cccc(-c8ccccc8)c7)n6)C5)CC4)CC3)cc21]

Synthesis of tert-butyl 2-(4-(1-(2,6-dioxopiperidin-3-yl)-3-methyl-2-oxo-2,3-dihydro-1H-benzo[d]imidazol-5-yl)piperidin-1-yl)acetate (051-2). To a solution of 3-(3-methyl-2-oxo-5-(piperidin-4-yl)-2,3-dihydro-1H-benzo[d]imidazol-1-yl)piperidine-2,6-dione (051-1) (235 mg, 1.21 mmol, 178 μL, 1.10 eq) and tert-butyl 2-bromoacetate (500 mg, 1.10 mmol, 1.00 eq, TFA) in DMF (5 mL) was added DIEA (566 mg, 4.38 mmol, 763 μL, 4.00 eq) at 25 °C. The mixture was stirred at 25 °C for 2 h. LCMS showed 3-(3-methyl-2-oxo-5-(piperidin-4-yl)-2,3-dihydro-1H-benzo[d]imidazol-1-yl)piperidine-2,6-dione (051-1) remained and the desired mass was detected. The resulting mixture was stirred at 25 °C for 3 h. LCMS showed 3-(3-methyl-2-oxo-5-(piperidin-4-yl)-2,3-dihydro-1H-benzo[d]imidazol-1-yl)piperidine-2,6-dione (051-1) was consumed and the desired mass was detected. H₂O (15 mL) was added to the solution, the mixture was extracted with EtOAc (15 mL*3), then the combined organic layers were washed with brine (15 mL*3), dried over Na₂SO₄, then concentrated in vacuo to give the crude product. The mixture was purified by prep-TLC (SiO₂, PE:EtOAc=0:1) to give the title compound (051-2) (220 mg, 482 μmol, 43.9% yield) as a white solid. LCMS (ES+): m/z 457.1 [M+H]+.

Synthesis of 2-(4-(1-(2,6-dioxopiperidin-3-yl)-3-methyl-2-oxo-2,3-dihydro-1H-benzo[d]imidazol-5-yl)piperidin-J-yl)acetic acid (051-3). The solution of tert-butyl 2-(4-(1-(2,6-dioxopiperidin-3-yl)-3-methyl-2-oxo-2,3-dihydro-1H-benzo[d]imidazol-5-yl)piperidin-1-yl)acetate (051-2) (200 mg, 438 μmol, 1.00 eq) in DCM (1.5 mL) and TFA (0.5 mL) was stirred at 25 °C for 1 h. LCMS showed tert-butyl 2-(4-(1-(2,6-dioxopiperidin-3-yl)-3-methyl-2-oxo-2,3-dihydro-1H-benzo[d]imidazol-5-yl)piperidin-1-yl)acetate (051-2) was consumed completely and the desired mass was detected. The mixture was concentrated in vacuo to give the title compound (051-3) (200 mg, crude) as a yellow oil. LCMS (ES+): m/z 401.2 [M+H]+.

Synthesis of tert-butyl 4-(2-(4-(1-(2,6-dioxopiperidin-3-yl)-3-methyl-2-oxo-2,3-dihydro-1H-benzo[d]imidazol-5-yl)piperidin-1-yl)acetyl)piperazine-1-carboxylate (051-4) To a solution of 2-(4-(1-(2,6-dioxopiperidin-3-yl)-3-methyl-2-oxo-2,3-dihydro-1H-benzo[d]imidazol-5-yl)piperidin-1-yl)acetic acid (051-3) (200 mg, 499 μmol, 1.00 eq) in DMF (3 mL) were added HATU (285 mg, 749 μmol, 1.50 eq), DIEA (194 mg, 1.50 mmol, 261 μL, 3.00 eq) and HOAt (102 mg, 749 μmol, 105 μL, 1.50 eq), then was added tert-butyl piperazine-1-carboxylate (111 mg, 499 μmol, 1.00 eq) at 25 °C. The mixture was stirred at 25 °C for 12 h. LCMS showed 2-(4-(1-(2,6-dioxopiperidin-3-yl)-3-methyl-2-oxo-2,3-dihydro-1H-benzo[d]imidazol-5-yl)piperidin-1-yl)acetic acid (051-3) was consumed completely and the desired mass was detected. H₂O (15 mL) was added to the solution, the mixture was extracted with EtOAc (10 mL*3), then the combined organic layers were washed with brine (15 mL*3), dried over Na₂SO₄, then concentrated in vacuo. The mixture was purified by prep-TLC (SiO₂, PE:EtOAc=1:5) to give the title compound (051-4) (110 mg, 193 μmol, 38.7% yield) as a white solid. LCMS (ES+): m/z 569.3 [M+H]+.

Synthesis of 3-(3-methyl-2-oxo-5-(1-(2-oxo-2-(piperazin-1-yl)ethyl)piperidin-4-yl)-2,3-dihydro-1H-benzo[d]imidazol-1-yl)piperidine-2,6-dione (051-5). The solution of tert-butyl 4-(2-(4-(1-(2,6-dioxopiperidin-3-yl)-3-methyl-2-oxo-2,3-dihydro-1H-benzo[d]imidazol-5-yl)piperidin-1-yl)acetyl)piperazine-1-carboxylate (051-4) (110 mg, 193 μmol, 1.00 eq) in DCM (2 mL) and TFA (0.4 mL) was stirred at 15 °C for 0.5 h. LCMS showed tert-butyl 4-(2-(4-(1-(2,6-dioxopiperidin-3-yl)-3-methyl-2-oxo-2,3-dihydro-1H-benzo[d]imidazol-5-yl)piperidin-1-yl)acetyl)piperazine-1-carboxylate (051-4) was consumed and the desired mass was detected. The mixture was concentrated in vacuo to give the title compound (051-5) (110 mg, crude, TFA) as a yellow oil. LCMS (ES+): m/z 469.3 [M+H]+.

Synthesis of cis-3-(5-(1-(2-(4-(3-((4-([1,1′-biphenyl]-3-yl)-5-chloropyrimidin-2-yl)amino)cyclohexane-1-carbonyl)piperazin-1-yl)-2-oxoethyl)piperidin-4-yl)-3-methyl-2-oxo-2,3-dihydro-1H-benzo[d]imidazol-1-yl)piperidine-2,6-dione (Compound 51). To a solution of 3-(3-methyl-2-oxo-5-(1-(2-oxo-2-(piperazin-1-yl)ethyl)piperidin-4-yl)-2,3-dihydro-1H-benzo[d]imidazol-1-yl)piperidine-2,6-dione (051-5) (100 mg, 172 μmol, 1.00 eq, TFA) and cis-3-((4-([1,1′-biphenyl]-3-yl)-5-chloropyrimidin-2-yl)amino)cyclohexane-1-carboxylic acid (70.0 mg, 172 μmol, 1.00 eq) (prepared by methods analogous to those shown in Example 22) in DMF (1 mL) were added HATU (97.9 mg, 258 μmol, 1.50 eq) and DIEA (66.6 mg, 515 μmol, 89.7 μL, 3.00 eq) at 25 °C. The mixture was stirred at 25 °C for 1 h. LCMS showed 3-(3-methyl-2-oxo-5-(1-(2-oxo-2-(piperazin-1-yl)ethyl)piperidin-4-yl)-2,3-dihydro-1H-benzo[d]imidazol-1-yl)piperidine-2,6-dione (051-5) was consumed completely and the desired mass was detected. The mixture was diluted with H₂O (10 mL) and then extracted with EtOAc (5 mL*3). The combined organic layers were dried over Na₂SO₄, filtered and the filtrate was concentrated in vacuo. The residue was purified by prep-HPLC (column: Phenomenex Luna C18 75*30 mm*3 μm; mobile phase: [water (FA)-ACN]; B %: 25%-65%, 8 min) to give the title compound (Compound 51) (17.1 mg, 19.1 μmol, 11.2% yield, 96.1% purity) as a white solid. LCMS (ES+): m/z 858.3 [M+H]+.

### Example 52. Synthesis of cis-3-(5-(1-(2-(1-(3-((4-([1,1′-biphenyl]-3-yl)-5-chloropyrimidin-2-yl)amino)cyclohexane-1-carbonyl)piperidin-4-yl)ethyl)piperidin-4-yl)-3-methyl-2-oxo-2,3-dihydro-1H-benzo[d]imidazol-1-yl)piperidine-2,6-dione (Compound 52)

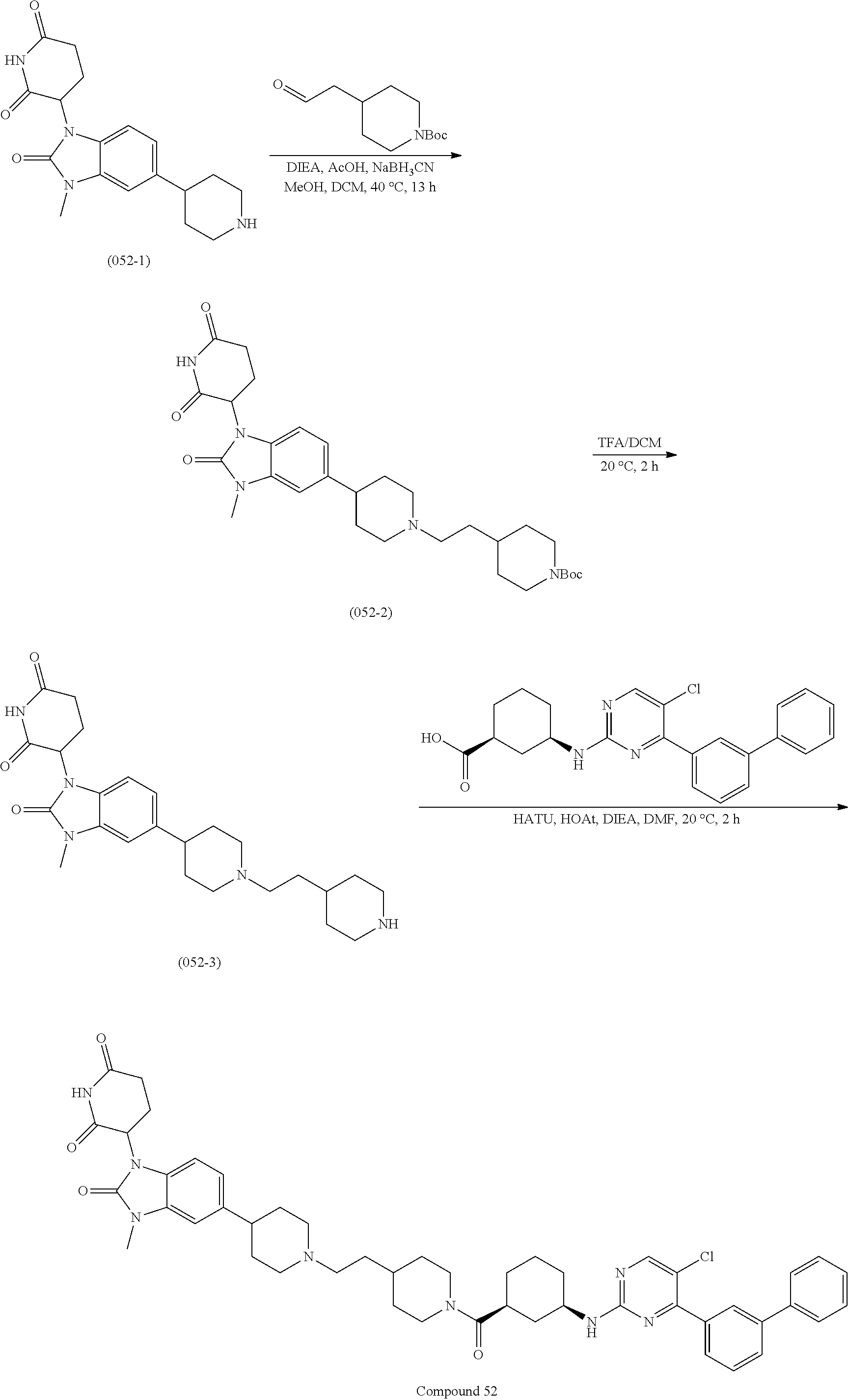 [SMILES: Cn1c(=O)n(C2CCC(=O)NC2=O)c2ccc(C3CCNCC3)cc21 ; CC(C)(C)OC(=O)N1CCC(CC=O)CC1 ; Cn1c(=O)n(C2CCC(=O)NC2=O)c2ccc(C3CCN(CCC4CCN(C(=O)OC(C)(C)C)CC4)CC3)cc21 ; Cn1c(=O)n(C2CCC(=O)NC2=O)c2ccc(C3CCN(CCC4CCNCC4)CC3)cc21 ; O=C(O)[C@H]1CCC[C@@H](Nc2ncc(Cl)c(-c3cccc(-c4ccccc4)c3)n2)C1 ; Cn1c(=O)n(C2CCC(=O)NC2=O)c2ccc(C3CCN(CCC4CCN(C(=O)[C@H]5CCC[C@@H](Nc6ncc(Cl)c(-c7cccc(-c8ccccc8)c7)n6)C5)CC4)CC3)cc21]

Synthesis of tert-butyl 4-(2-(4-(1-(2,6-dioxopiperidin-3-yl)-3-methyl-2-oxo-2,3-dihydro-1H-benzo[d]imidazol-5-yl)piperidin-1-yl)ethyl)piperidine-1-carboxylate (052-2). To a stirred solution of 3-(3-methyl-2-oxo-5-(piperidin-4-yl)-2,3-dihydro-1H-benzo[d]imidazol-1-yl)piperidine-2,6-dione (052-1) (450 mg, 986 μmol, 1.00 eq, TFA) in MeOH (6 mL) and DCM (3 mL) was added DIEA (127 mg, 986 μmol, 172 μL, 1.00 eq) at 25 °C. The mixture was stirred at 25 °C for 0.5 h. Then to the mixture was added tert-butyl 4-(2-oxoethyl)piperidine-1-carboxylate (269 mg, 1.18 mmol, 1.20 eq) and AcOH (5.92 mg, 98.6 mol, 5.64 μL, 0.100 eq) at 25 °C. The mixture was stirred at 40 °C for 0.5 h. Then to the mixture was added NaBH₃CN (186 mg, 2.96 mmol, 3.00 eq) at 25 °C. The resulting mixture was stirred at 40 °C for 12 h. LCMS showed 3-(3-methyl-2-oxo-5-(piperidin-4-yl)-2,3-dihydro-1H-benzo[d]imidazol-1-yl)piperidine-2,6-dione (052-1) was consumed and the desired mass was detected. The mixture was diluted with H₂O (10 mL) and then extracted with EtOAc (5 mL*3). The combined organic layers were dried over Na₂SO₄, filtered and the filtrate was concentrated in vacuo. The mixture was purified by prep-TLC (SiO₂, DCM:MeOH=10:1) to give the title compound (052-2) (450 mg, 813 μmol, 82.4% yield) as a yellow oil. LCMS (ES+): m/z 554.3 [M+H]+.

Synthesis of 3-(3-methyl-2-oxo-5-(1-(2-(piperidin-4-yl)ethyl)piperidin-4-yl)-2,3-dihydro-1H-benzo[d]imidazol-1-yl)piperidine-2,6-dione (052-3). To a solution tert-butyl 4-(2-(4-(1-(2,6-dioxopiperidin-3-yl)-3-methyl-2-oxo-2,3-dihydro-1H-benzo[d]imidazol-5-yl)piperidin-1-yl)ethyl)piperidine-1-carboxylate (052-2) (450 mg, 813 μmol, 1.00 eq) in DCM (3 mL) was added TFA (0.5 mL) at 20 °C. The mixture was stirred at 20 °C for 2 h. LCMS showed that tert-butyl 4-(2-(4-(1-(2,6-dioxopiperidin-3-yl)-3-methyl-2-oxo-2,3-dihydro-1H-benzo[d]imidazol-5-yl)piperidin-1-yl)ethyl)piperidine-1-carboxylate (052-2) was consumed completely and the desired mass was detected. The mixture was concentrated in vacuo to give the title compound (052-3) (400 mg, crude, TFA) as a yellow oil. LCMS (ES+): m/z 454.3 [M+H]+.

Synthesis of cis-3-(5-(1-(2-(1-(3-((4-([1,1′-biphenyl]-3-yl)-5-chloropyrimidin-2-yl)amino)cyclohexane-1-carbonyl)piperidin-4-yl)ethyl)piperidin-4-yl)-3-methyl-2-oxo-2,3-dihydro-1H-benzo[d]imidazol-1-yl)piperidine-2,6-dione (Compound 52). To a solution of cis-3-((4-([1,1′-biphenyl]-3-yl)-5-chloropyrimidin-2-yl)amino)cyclohexane-1-carboxylic acid (201 mg, 493 μmol, 1.00 eq) in DMF (5 mL) were added HOAt (33.6 mg, 247 μmol, 34.5 μL, 0.500 eq), HATU (281 mg, 740 μmol, 1.50 eq) and DIEA (319 mg, 2.47 mmol, 430 μL, 5.00 eq) at 20 °C, 3-(3-methyl-2-oxo-5-(1-(2-(piperidin-4-yl)ethyl)piperidin-4-yl)-2,3-dihydro-1H-benzo[d]imidazol-1-yl)piperidine-2,6-dione (052-3) (400 mg, 493 μmol, 1.00 eq, TFA) was added to the mixture, the mixture was stirred at 20 °C for 2 h. LCMS showed that 3-(3-methyl-2-oxo-5-(1-(2-(piperidin-4-yl)ethyl)piperidin-4-yl)-2,3-dihydro-1H-benzo[d]imidazol-1-yl)piperidine-2,6-dione (052-3) was consumed completely the desired mass was detected. H₂O (3 mL) was added to the solution, then the mixture was extracted with EtOAc (3 mL*3), the combined organic layers were dried over Na₂SO₄, then concentrated in vacuo to give the crude product. The residue was purified by prep-HPLC (column: Phenomenex Luna C18 75*30 mm*3 μm; mobile phase: [water (FA)-ACN]; B %: 30%-70%, 8 min) to give the title compound (Compound 52) (20.4 mg, 22.9 μmol, 4.65% yield, 94.8% purity) as a white solid. LCMS (ES+): m/z 843.4 [M+H]+.

### Example 53. Synthesis of 3-((4-(1-((1-(2-(4-((4-([1,1′-biphenyl]-3-yl)-5-chloropyrimidin-2-yl)amino)piperidin-1-yl)-2-oxoethyl)piperidin-4-yl)methyl)piperidin-4-yl)phenyl)amino)piperidine-2,6-dione (Compound 53)

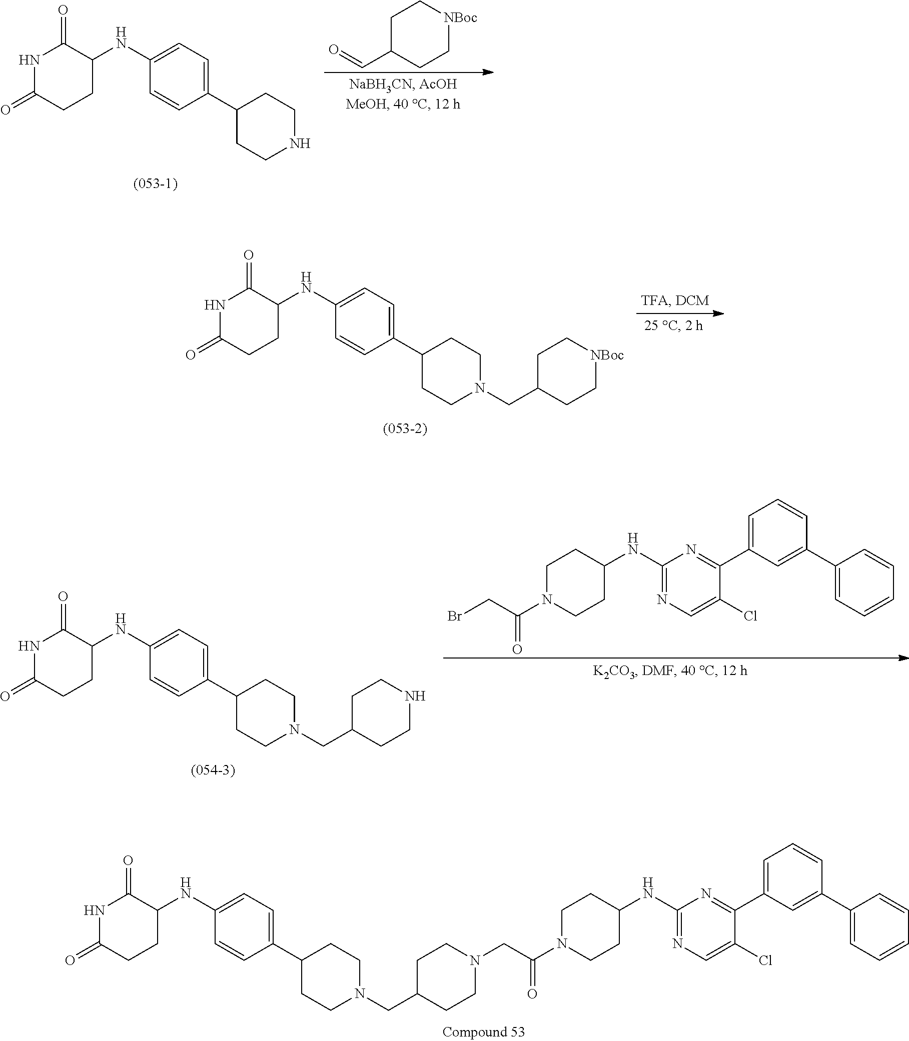 [SMILES: O=C1CCC(Nc2ccc(C3CCNCC3)cc2)C(=O)N1 ; CC(C)(C)OC(=O)N1CCC(C=O)CC1 ; CC(C)(C)OC(=O)N1CCC(CN2CCC(c3ccc(NC4CCC(=O)NC4=O)cc3)CC2)CC1 ; O=C1CCC(Nc2ccc(C3CCN(CC4CCNCC4)CC3)cc2)C(=O)N1 ; O=C(CBr)N1CCC(Nc2ncc(Cl)c(-c3cccc(-c4ccccc4)c3)n2)CC1 ; O=C1CCC(Nc2ccc(C3CCN(CC4CCN(CC(=O)N5CCC(Nc6ncc(Cl)c(-c7cccc(-c8ccccc8)c7)n6)CC5)CC4)CC3)cc2)C(=O)N1]

Synthesis of tert-butyl 4-((4-(4-((2,6-dioxopiperidin-3-yl)amino)phenyl)piperidin-1-yl)methyl)piperidine-1-carboxylate (053-2). To a solution of 3-((4-(piperidin-4-yl)phenyl)amino)piperidine-2,6-dione (053-1) (500 mg, 1.74 mmol, 1.00 eq) (prepared by methods known in the art), tert-butyl 4-formylpiperidine-1-carboxylate (482 mg, 2.26 mmol, 1.30 eq) and HOAc (522 mg, 8.70 mmol, 498 μL, 5.00 eq) in MeOH (10 mL) was added NaBH₃CN (219 mg, 3.48 mmol, 2.00 eq) at 25 °C. The mixture was stirred at 40 °C for 12 h. LCMS showed 3-((4-(piperidin-4-yl)phenyl)amino)piperidine-2,6-dione (053-1) was consumed completely and desired mass was detected. Water (20 mL) was added to the solution, the mixture was extracted with ethyl acetate (20 mL*3), the combined organic layers were dried over Na₂SO₄, and concentrated under reduced pressure. The residue was purified by column chromatography (SiO₂, Dichloromethane:Methanol=10:1 to 5:1) to give the title compound (053-2) (350 mg, 433 μmol, 24.9% yield, 60.0% purity) as a white solid. LCMS (ES+): m/z 485.3 [M+H]+.

Synthesis of 3-((4-(1-(piperidin-4-ylmethyl)piperidin-4-yl)phenyl)amino)piperidine-2,6-dione (053-3). To a solution of tert-butyl 4-((4-(4-((2,6-dioxopiperidin-3-yl)amino)phenyl)piperidin-1-yl)methyl)piperidine-1-carboxylate (053-2) (350 mg, 722 μmol, 1.00 eq) in DCM (6 mL) was added TFA (3.08 g, 27.0 mmol, 2.00 mL, 37.4 eq) at 25 °C. The mixture was stirred at 25 °C for 2 h. LCMS showed tert-butyl 4-((4-(4-((2,6-dioxopiperidin-3-yl)amino)phenyl)piperidin-1-yl)methyl)piperidine-1-carboxylate (053-2) was consumed completely and the desired mass was detected. The reaction concentrated under reduced pressure to give the title compound (053-3) (700 mg, crude, TFA) as a white solid. LCMS (ES+): m/z 385.3 [M+H]+.

Synthesis of 3-((4-(1-((1-(2-(4-((4-([1,1′-biphenyl]-3-yl)-5-chloropyrimidin-2-yl)amino)piperidin-1-yl)-2-oxoethyl)piperidin-4-yl)methyl)piperidin-4-yl)phenyl)amino)piperidine-2,6-dione (Compound 53). To a solution of 3-((4-(1-(piperidin-4-ylmethyl)piperidin-4-yl)phenyl)amino)piperidine-2,6-dione (053-3) (100 mg, 260 μmol, 1.00 eq) in DMF (2 mL) were added K₂CO₃ (180 mg, 1.30 mmol, 5.00 eq) and 2-bromo-1-[4-[[5-chloro-4-(3-phenylphenyl)pyrimidin-2-yl]amino]-1-piperidyl]ethanone (126 mg, 260 μmol, 1.00 eq) (prepared as described in Example 83) at 25 °C. The mixture was stirred at 40 °C for 12 h. LCMS showed 3-((4-(1-(piperidin-4-ylmethyl)piperidin-4-yl)phenyl)amino)piperidine-2,6-dione (053-3) was consumed completely and the desired mass was detected. The reaction filtered and concentrated under reduced pressure. The residue was purified by prep-HPLC (column: Phenomenex Luna C18 75*30 mm*3 μm; mobile phase: [water (FA)-ACN]; B %: 20%-60%, 8 min) to give the title compound (Compound 53) (84.4 mg, 101 μmol, 38.7% yield, 96.0% purity, 0.32 FA) as a light green solid. LCMS (ES+): m/z 789.3 [M+H]+.

### Example 54. Synthesis of cis-3-(5-(1-(2-(4-(3-((4-([1,1′-biphenyl]-3-yl)-5-chloropyrimidin-2-yl)amino)cyclohexane-1-carbonyl)piperazin-1-yl)-2-oxoethyl)piperidin-4-yl)-1-oxoisoindolin-2-yl)piperidine-2,6-dione (Compound 54)

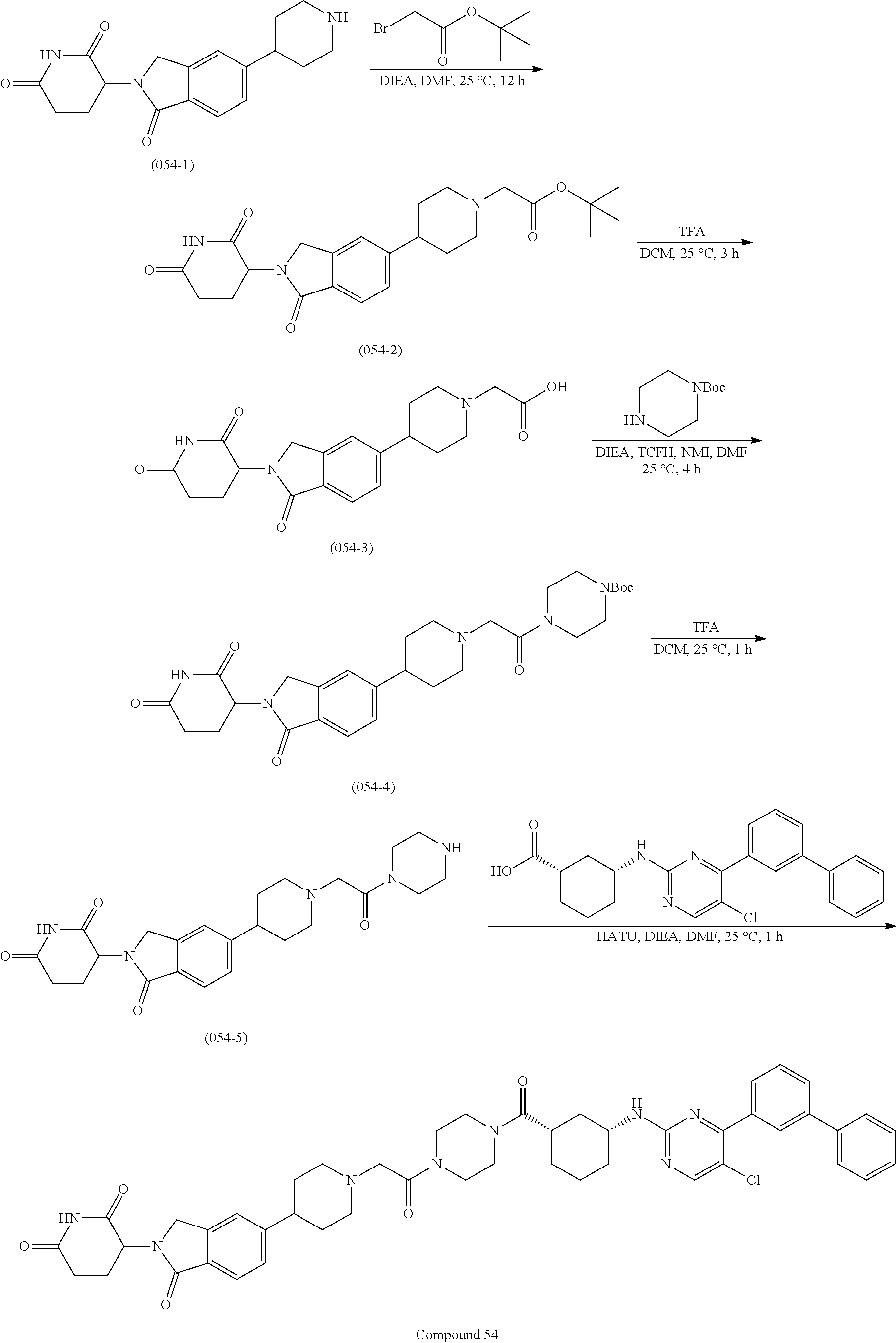

Synthesis of tert-butyl 2-(4-(2-(2,6-dioxopiperidin-3-yl)-1-oxoisoindolin-5-yl)piperidin-1-yl)acetate (054-2). To a solution of 3-(1-oxo-5-(piperidin-4-yl)isoindolin-2-yl)piperidine-2,6-dione (054-1) (300 mg, 916 μmol, 1.00 eq) (prepared by methods known in the art) in DMF (3 mL) were added DIEA (474 mg, 3.67 mmol, 638 μL, 4.00 eq) and tert-butyl 2-bromoacetate (197 mg, 1.01 mmol, 149 μL, 1.10 eq) at 25 °C. The mixture was stirred at 25 °C for 12 h. LCMS showed 3-(1-oxo-5-(piperidin-4-yl)isoindolin-2-yl)piperidine-2,6-dione (054-1) was consumed completely and the desired mass was detected. Water (5 mL) was added to the mixture at 25 °C. Then the reaction mixture was extracted with EtOAc (5 mL*3). The organic layers were dried over anhydrous sodium sulfate, filtered and the filtrate was concentrated under reduced pressure to give a residue. The residue was purified by prep-TLC (SiO₂, Ethyl acetate:Methanol=7:1) to give the title compound (054-2) (240 mg, 408 μmol, 44.5% yield, 75.0% purity) as a white solid. LCMS (ES+): m/z 442.3 [M+H]+.

Synthesis of 2-(4-(2-(2,6-dioxopiperidin-3-yl)-1-oxoisoindolin-5-yl)piperidin-1-yl)acetic acid (054-3). To a solution of tert-butyl 2-(4-(2-(2,6-dioxopiperidin-3-yl)-1-oxoisoindolin-5-yl)piperidin-1-yl)acetate (054-2) (430 mg, 974 μmol, 1.00 eq) in DCM (5 mL) was added TFA (111 mg, 974 μmol, 72.1 μL, 1.00 eq) at 25 °C. The mixture was stirred at 25 °C for 3 h. LCMS showed tert-butyl 2-(4-(2-(2,6-dioxopiperidin-3-yl)-1-oxoisoindolin-5-yl)piperidin-1-yl)acetate (054-2) was consumed completely and the desired mass was detected. The reaction mixture was concentrated under reduced pressure to remove solvent to give the title compound (054-3) (240 mg, crude) as a colorless oil. LCMS (ES+): m/z 386.3 [M+H]+.

Synthesis of tert-butyl 4-(2-(4-(2-(2,6-dioxopiperidin-3-yl)-1-oxoisoindolin-5-yl)piperidin-1-yl)acetyl)piperazine-1-carboxylate (054-4). To a solution of 2-(4-(2-(2,6-dioxopiperidin-3-yl)-1-oxoisoindolin-5-yl)piperidin-1-yl)acetic acid (054-3) (240 mg, 623 μmol, 1.00 eq) in DMF (3 mL) were added NMI (56.2 mg, 685 μmol, 54.6 μL, 1.10 eq), DIEA (402 mg, 3.11 mmol, 542 μL, 5.00 eq), tert-butyl piperazine-1-carboxylate hydrochloride (166 mg, 747 μmol, 1.20 eq) and TCFH (524 mg, 1.87 mmol, 3.00 eq) at 25 °C. The mixture was stirred at 25 °C for 4 h. LCMS showed 2-(4-(2-(2,6-dioxopiperidin-3-yl)-1-oxoisoindolin-5-yl)piperidin-1-yl)acetic acid (054-3) was consumed completely and the desired mass was detected. Water (2 mL) was added to the mixture at 25 °C. Then the reaction mixture was extracted with EtOAc (1 mL*3). The organic layers were dried over anhydrous sodium sulfate, filtered and the filtrate was concentrated under reduced pressure to give a residue. The residue was purified by prep-TLC (SiO₂, Ethyl acetate:Methanol=8:1) to give the title compound (054-4) (240 mg, 309 μmol, 49.7% yield, 71.4% purity) as a white solid. LCMS (ES+): m/z 554.1 [M+H]+.

Synthesis of 3-(1-oxo-5-(1-(2-oxo-2-(piperazin-1-yl)ethyl)piperidin-4-yl)isoindolin-2-yl)piperidine-2,6-dione (054-5). To a solution of tert-butyl 4-(2-(4-(2-(2,6-dioxopiperidin-3-yl)-1-oxoisoindolin-5-yl)piperidin-1-yl)acetyl)piperazine-1-carboxylate (054-4) (230 mg, 415 μmol, 1.00 eq) in DCM (3 mL) was added TFA (1.54 g, 13.5 mmol, 1.00 mL, 32.5 eq) at 25 °C. The mixture was stirred at 25 °C for 1 h. LCMS showed tert-butyl 4-(2-(4-(2-(2,6-dioxopiperidin-3-yl)-1-oxoisoindolin-5-yl)piperidin-1-yl)acetyl)piperazine-1-carboxylate (054-4) was consumed completely and the desired mass was detected. The reaction mixture was concentrated under reduced pressure to remove solvent to give the title compound (054-5) (225 mg, crude, TFA) as a brown oil. LCMS (ES+): m/z 454.1 [M+H]+.

Synthesis of cis-3-(5-(1-(2-(4-(3-((4-([1,1′-biphenyl]-3-yl)-5-chloropyrimidin-2-yl)amino)cyclohexane-1-carbonyl)piperazin-1-yl)-2-oxoethyl)piperidin-4-yl)-1-oxoisoindolin-2-yl)piperidine-2,6-dione (Compound 54). To a solution of 3-(1-oxo-5-(1-(2-oxo-2-(piperazin-1-yl)ethyl)piperidin-4-yl)isoindolin-2-yl)piperidine-2,6-dione (054-5) (225 mg, 396 μmol, 1.00 eq, TFA) in DMF (3 mL) were added DIEA (256 mg, 1.98 mmol, 345 μL, 5.00 eq), cis-3-((4-([1,1′-biphenyl]-3-yl)-5-chloropyrimidin-2-yl)amino)cyclohexane-1-carboxylic acid (141 mg, 317 μmol, 0.800 eq) (prepared by methods analogous to those shown in Example 22) and HATU (226 mg, 595 μmol, 1.50 eq) at 25 °C. The mixture was stirred at 25 °C for 1 h. LCMS showed 3-(1-oxo-5-(1-(2-oxo-2-(piperazin-1-yl)ethyl)piperidin-4-yl)isoindolin-2-yl)piperidine-2,6-dione (054-5) was consumed completely and the desired mass was detected. The reaction mixture was concentrated under reduced pressure to remove solvent. The residue were purified by prep-HPLC (column: Phenomenex Luna C18 200*40 mm*10 μm; mobile phase: [water (FA)-ACN]; B %: 30%-65%, 8 min) to give the title compound (Compound 54) (42.7 mg, 50.0 μmol, 12.6% yield, 98.7% purity) as a white solid. LCMS (ES+): m/z 843.2 [M+H]+.

### Example 55. Synthesis of cis-3-((4-(1-(2-(4-(3-((4-([1,1′-biphenyl]-3-yl)-5-chloropyrimidin-2-yl)amino)cyclohexane-1-carbonyl)piperazin-1-yl)ethyl)piperidin-4-yl)phenyl)amino)piperidine-2,6-dione (Compound 55)

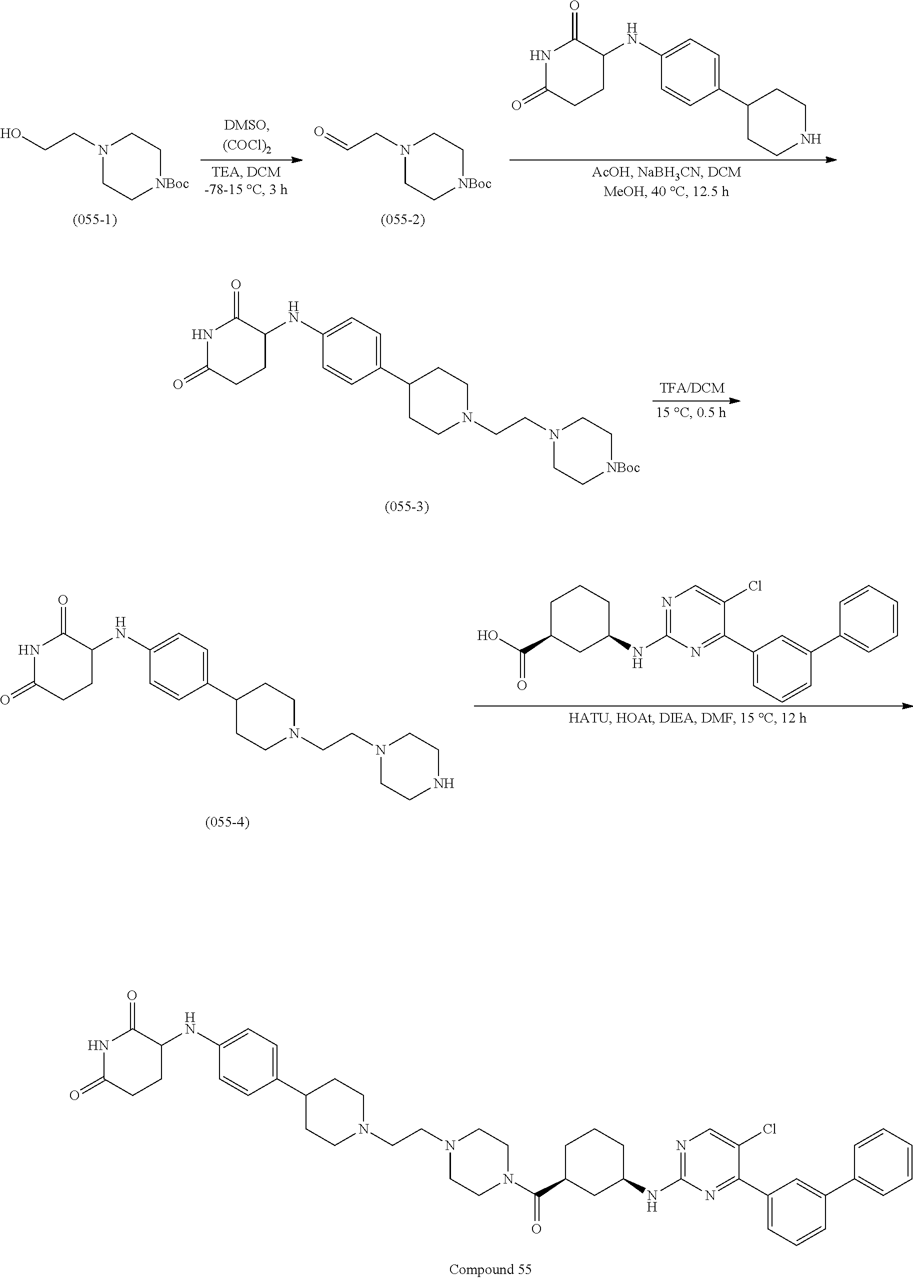

Synthesis of tert-butyl 4-(2-oxoethyl)piperazine-1-carboxylate. To a stirred solution of (COCl)₂ (1.10 g, 8.67 mmol, 759 μL, 2.00 eq) in DCM (15 mL) was added DMSO (1.24 g, 15.9 mmol, 1.24 mL, 3.65 eq) at −78 °C and the mixture was stirred at −78 °C for 0.5 h. To the mixture was added tert-butyl 4-(2-hydroxyethyl)piperazine-1-carboxylate (055-1) (1.00 g, 4.34 mmol, 1.00 eq) in DCM (4 mL) at −78 °C and the mixture was stirred at −78 °C for 0.5 h. Then to the mixture was added TEA (2.65 g, 26.2 mmol, 3.64 mL, 6.02 eq) at −78 °C and the mixture was at 15 °C for 2 h. LCMS showed tert-butyl 4-(2-hydroxyethyl)piperazine-1-carboxylate (055-1) was consumed and the desired mass was detected. The mixture was diluted with saturated NaHCO₃ (20 mL) and then extracted with DCM (10 mL*3). The combined organic layers were dried over Na₂SO₄, filtered and the filtrate was concentrated in vacuo to give the title compound (055-2) (1.00 g, crude) as a yellow oil. LCMS (ES+): m/z 229.2 [M+H]+.

Synthesis of tert-butyl 4-(2-(4-(4-((2,6-dioxopiperidin-3-yl)amino)phenyl)piperidin-1-yl)ethyl)piperazine-1-carboxylate (055-3). To a stirred solution of 3-((4-(piperidin-4-yl)phenyl)amino)piperidine-2,6-dione (263 mg, 655 μmol, 1.00 eq, TFA) and DIEA (84.7 mg, 655 μmol, 114 μL, 1.00 eq) in DCM (2 mL) and MeOH (4 mL) were added tert-butyl 4-(2-oxoethyl)piperazine-1-carboxylate (055-2) (300 mg, 1.31 mmol, 2.00 eq) and AcOH (3.93 mg, 65.5 μmol, 3.75 μL, 0.100 eq) at 15 °C. The mixture was stirred at 40 °C for 0.5 h. Then to the mixture was added NaBH₃CN (82.3 mg, 1.31 mmol, 2.00 eq) at 15 °C. The resulting mixture was stirred at 40 °C for 12 h. LCMS showed tert-butyl 4-(2-oxoethyl)piperazine-1-carboxylate (055-2) consumed and the desired mass was detected. The mixture was diluted with H₂O (10 mL) and then extracted with EtOAc (5 mL*3). The combined organic layers were dried over Na₂SO₄, filtered and the filtrate was concentrated in vacuo. The mixture was purified by prep-TLC (SiO₂, DCM:MeOH=10:1) to give the title compound (055-3) (150 mg, 300 μmol, 45.8% yield) as a yellow solid. LCMS (ES+): m/z 500.3 [M+H]+.

Synthesis of 3-((4-(1-(2-(piperazin-1-yl)ethyl)piperidin-4-yl)phenyl)amino)piperidine-2,6-dione (055-4). A solution of tert-butyl 4-(2-(4-(4-((2,6-dioxopiperidin-3-yl)amino)phenyl)piperidin-1-yl)ethyl)piperazine-1-carboxylate (055-3) (150 mg, 300.21 μmol, 1.00 eq) in TFA (0.5 mL) and DCM (1.5 mL) was stirred at 15 °C for 0.5 h. LCMS showed tert-butyl 4-(2-(4-(4-((2,6-dioxopiperidin-3-yl)amino)phenyl)piperidin-1-yl)ethyl)piperazine-1-carboxylate (055-3) was consumed and the desired mass was detected. The mixture was concentrated in vacuo to give the title compound (055-4) (150 mg, crude, TFA) as a yellow oil. LCMS (ES+): m/z 400.3 [M+H]+.

Synthesis of cis-3-((4-(1-(2-(4-(3-((4-([1,1′-biphenyl]-3-yl)-5-chloropyrimidin-2-yl)amino)cyclohexane-1-carbonyl)piperazin-1-yl)ethyl)piperidin-4-yl)phenyl)amino)piperidine-2,6-dione (Compound 55). To a stirred solution of cis-3-((4-([1,1′-biphenyl]-3-yl)-5-chloropyrimidin-2-yl)amino)cyclohexane-1-carboxylic acid (119 mg, 292 μmol, 1.00 eq) (prepared by methods analogous to those shown in Example 22) and DIEA (113 mg, 876 μmol, 153 μL, 3.00 eq) in DMF (3 mL) were added HATU (167 mg, 438 mol, 1.50 eq), HOAt (59.6 mg, 438 μmol, 61.3 μL, 1.50 eq) and 3-((4-(1-(2-(piperazin-1-yl)ethyl)piperidin-4-yl)phenyl)amino)piperidine-2,6-dione (055-4) (150 mg, 292 μmol, 1.00 eq, TFA) at 15 °C. The resulting mixture was stirred at 15 °C for 12 h. LCMS showed 3-((4-(1-(2-(piperazin-1-yl)ethyl)piperidin-4-yl)phenyl)amino)piperidine-2,6-dione (055-4) was consumed and the desired mass was detected. The mixture was concentrated in vacuo. The crude product was purified by prep-HPLC (column: Phenomenex luna C18 100*40 mm*3 μm; mobile phase: [water (TFA)-ACN]; B %: 20%-63%, 8 min) to give the title compound (Compound 55) (2.60 mg, 3.11 μmol, 1.06% yield, 94.3% purity) as a green solid. LCMS (ES+): m/z 789.4 [M+H]+.

### Example 56. Synthesis of 3-(5-(1-(2-(4-(4-((4-([1,1′-biphenyl]-3-yl)-5-chloropyrimidin-2-yl)amino)piperidine-1-carbonyl)piperidin-1-yl)acetyl)piperidin-4-yl)-1-oxoisoindolin-2-yl)piperidine-2,6-dione (Compound 56)

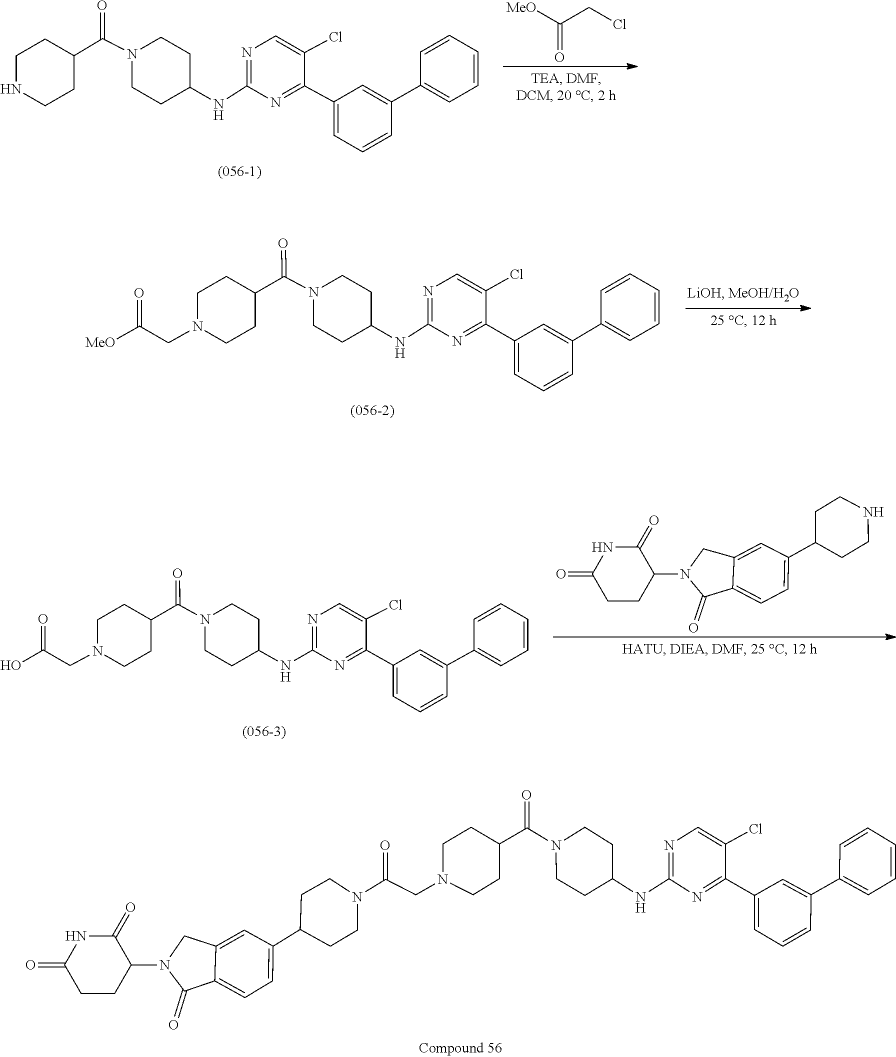 [SMILES: O=C(C1CCNCC1)N1CCC(Nc2ncc(Cl)c(-c3cccc(-c4ccccc4)c3)n2)CC1 ; COC(=O)CCl ; COC(=O)CN1CCC(C(=O)N2CCC(Nc3ncc(Cl)c(-c4cccc(-c5ccccc5)c4)n3)CC2)CC1 ; O=C(O)CN1CCC(C(=O)N2CCC(Nc3ncc(Cl)c(-c4cccc(-c5ccccc5)c4)n3)CC2)CC1 ; O=C1CCC(N2Cc3cc(C4CCNCC4)ccc3C2=O)C(=O)N1 ; O=C1CCC(N2Cc3cc(C4CCN(C(=O)CN5CCC(C(=O)N6CCC(Nc7ncc(Cl)c(-c8cccc(-c9ccccc9)c8)n7)CC6)CC5)CC4)ccc3C2=O)C(=O)N1]

Synthesis of methyl 2-(4-(4-((4-([1,1′-biphenyl]-3-yl)-5-chloropyrimidin-2-yl)amino)piperidine-1-carbonyl)piperidin-1-yl)acetate (056-2). To a solution of (4-((4-([1,1′-biphenyl]-3-yl)-5-chloropyrimidin-2-yl)amino)piperidin-1-yl)(piperidin-4-yl)methanone (Compound 2) (056-1) (1.00 g, 2.10 mmol, 1.00 eq) in DMF (2 mL) and DCM (10 mL) were added TEA (1.06 g, 10.5 mmol, 1.46 mL, 5.00 eq) and methyl 2-chloroacetate (274 mg, 2.52 mmol, 222 μL, 1.20 eq) at 20 °C. The mixture was stirred at 20 °C for 2 h under N₂. LCMS showed (4-((4-([1,1′-biphenyl]-3-yl)-5-chloropyrimidin-2-yl)amino)piperidin-1-yl)(piperidin-4-yl)methanone (Compound 2) (056-1) was consumed completely and the desired mass was detected. The reaction mixture was diluted with H₂O (40 mL) and extracted with EtOAc (30 mL*3). The combined organic layers were dried over Na₂SO₄, filtered and concentrated under reduced pressure to give a residue. The residue was purified by column chromatography (SiO₂, DCM:MeOH=1:0 to 4:1) to give the title compound (056-2) (530 mg, 958 μmol, 99.1% yield) as yellow solid. LCMS (ES+): m/z 548.2 [M+H]+.

Synthesis of 2-(4-(4-((4-([1,1′-biphenyl]-3-yl)-5-chloropyrimidin-2-yl)amino)piperidine-1-carbonyl)piperidin-1-yl)acetic acid (056-3). To a solution of methyl 2-(4-(4-((4-([1,1′-biphenyl]-3-yl)-5-chloropyrimidin-2-yl)amino)piperidine-1-carbonyl)piperidin-1-yl)acetate (056-2) (530 mg, 967 μmol, 1.00 eq) in MeOH (6 mL) and H₂O (2 mL) was added LiOH (46.3 mg, 1.93 mmol, 2.00 eq) at 25 °C. The mixture was stirred at 25 °C for 12 h. LCMS showed methyl 2-(4-(4-((4-([1,1′-biphenyl]-3-yl)-5-chloropyrimidin-2-yl)amino)piperidine-1-carbonyl)piperidin-1-yl)acetate (056-2) was consumed completely and the desired mass was detected. The mixture was diluted with H₂O (40 mL), adjusted pH to 5 with 1 M HCl solution and extracted with EtOAc (30 mL*3). The combined organic layers were dried over Na₂SO₄, filtered and concentrated under reduced pressure to give the title compound (056-3) (480 mg, 852 μmol, 94.8% yield) as a yellow solid. LCMS (ES+): m/z 534.2 [M+H]+.

Synthesis of (Compound 56). To a solution of 2-(4-(4-((4-([1,1′-biphenyl]-3-yl)-5-chloropyrimidin-2-yl)amino)piperidine-1-carbonyl)piperidin-1-yl)acetic acid (056-3) (100 mg, 187 μmol, 1.00 eq) in DMF (5 mL) were added HATU (107 mg, 2801 μmol, 1.50 eq), 3-[1-oxo-5-(4-piperidyl)isoindolin-2-yl]piperidine-2,6-dione (73.6 mg, 225 μmol, 1.20 eq) and DIEA (121 mg, 936 μmol, 163 μL, 5.00 eq) at 25 °C. The mixture was stirred at 25 °C for 12 h. LCMS showed 2-(4-(4-((4-([1,1′-biphenyl]-3-yl)-5-chloropyrimidin-2-yl)amino)piperidine-1-carbonyl)piperidin-1-yl)acetic acid (056-3) was consumed completely and the desired mass was detected. The reaction mixture was concentrated under reduced pressure. The residue was purified by prep-HPLC (column: Phenomenex luna C18 80*40 mm*3 μm; mobile phase: [water (FA)-ACN]; B %: 30%-60%, 8 min) to give the title compound (Compound 56) (42.9 mg, 48.5 μmol, 25.9% yield, 100% purity, 0.9 FA) as a white solid. LCMS (ES+): m/z 843.3 [M+H]+.

### Example 57. Synthesis of cis-3-((4-(1-(2-(4-(3-((4-([1,1′-biphenyl]-3-yl)-5-chloropyrimidin-2-yl)amino)cyclohexane-1-carbonyl)piperazin-1-yl)-2-oxoethyl)piperidin-4-yl)phenyl)amino)piperidine-2,6-dione (Compound 57)

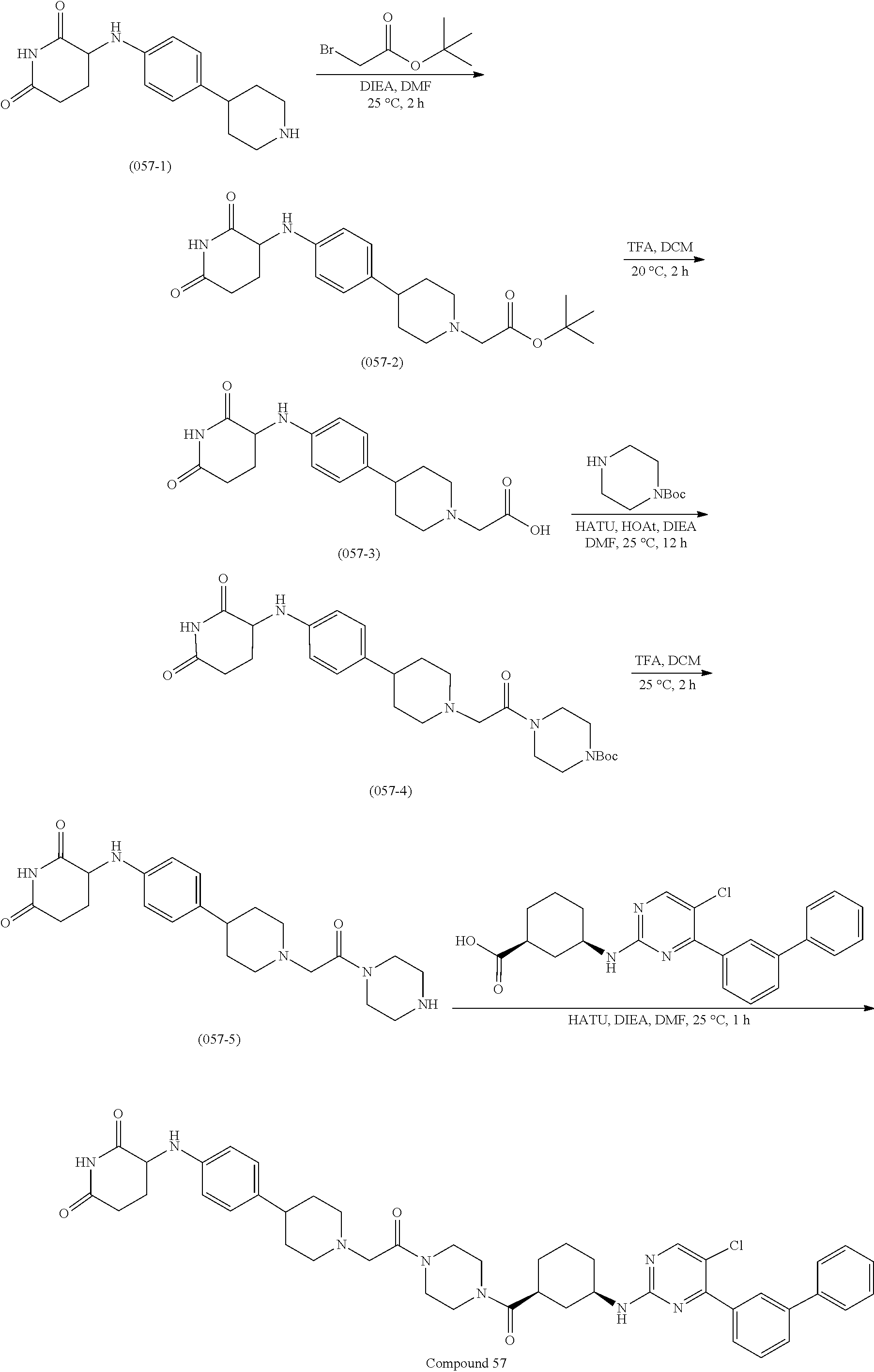

Synthesis of tert-butyl 2-(4-(4-((2,6-dioxopiperidin-3-yl)amino)phenyl)piperidin-1-yl)acetate (057-2). To a solution of 3-((4-(piperidin-4-yl)phenyl)amino)piperidine-2,6-dione (057-1) (300 mg, 1.04 mmol, 1.00 eq) and DIEA (540 mg, 4.18 mmol, 7.27 μL, 4.00 eq) in DMF (6 mL) was added tert-butyl 2-bromoacetate (224 mg, 1.15 mmol, 170 μL, 1.10 eq) at 25 °C. The mixture was stirred at 25 °C for 2 h. LCMS showed 3-((4-(piperidin-4-yl)phenyl)amino)piperidine-2,6-dione (057-1) consumed and the desired mass was detected. The mixture was diluted with H₂O (10 mL) and then extracted with EtOAc (10 mL*3). The combined organic layers were washed with saturated aqueous NaCl solution (20 mL*3), dried over Na₂SO₄ and then concentrated in vacuo. The mixture was purified by prep-TLC (SiO₂, PE:EtOAc=0:1) to give the title compound (057-2) (200 mg, 498 μmol, 47.7% yield) as a white solid. LCMS (ES+): m/z 402.1 [M+H]+.

Synthesis of 2-(4-(4-((2,6-dioxopiperidin-3-yl)amino)phenyl)piperidin-1-yl)acetic acid (057-3). The solution of tert-butyl 2-(4-(4-((2,6-dioxopiperidin-3-yl)amino)phenyl)piperidin-1-yl)acetate (057-2) (200 mg, 498 mmol, 1.00 eq) in DCM (3 mL) and TFA (1 mL) was stirred at 20 °C for 2 h. LCMS showed tert-butyl 2-(4-(4-((2,6-dioxopiperidin-3-yl)amino)phenyl)piperidin-1-yl)acetate (057-2) was consumed and the desired mass was detected. The mixture was concentrated in vacuo to give the title compound (057-3) (200 mg, crude, TFA) as a yellow solid. LCMS (ES+): m/z 346.0 [M+H]+.

Synthesis of tert-butyl 4-(2-(4-(4-((2,6-dioxopiperidin-3-yl)amino)phenyl)piperidin-1-yl)acetyl)piperazine-1-carboxylate (057-4). To a solution of 2-(4-(4-((2,6-dioxopiperidin-3-yl)amino)phenyl)piperidin-1-yl)acetic acid (057-3) (200 mg, 435 μmol, 1.00 eq, TFA) in DMF (3 mL) were added HATU (248 mg, 653 μmol, 1.50 eq), DIEA (169 mg, 1.31 mmol, 227 μL, 3.00 eq) and HOAt (88.9 mg, 653. μmol, 91.4 μL, 1.50 eq), then was added tert-butyl piperazine-1-carboxylate (116 mg, 522 μmol, 1.20 eq, HCl) at 25 °C. The mixture was stirred at 25 °C for 12 h. LCMS showed 2-(4-(4-((2,6-dioxopiperidin-3-yl)amino)phenyl)piperidin-1-yl)acetic acid (057-3) was consumed and the desired mass was detected. To the mixture was added H₂O (10 mL) and the mixture was extracted with EtOAc (15 mL*3). The combined organic layers were washed with saturated aqueous NaCl solution (20 mL*3), dried over Na₂SO₄ and then concentrated in vacuo. The mixture was purified by prep-TLC (SiO₂, PE:EtOAc=1:5) to give the title compound (057-4) (200 mg, 389 μmol, 89.5% yield) as a white solid. LCMS (ES+): m/z 514.3 [M+H]+.

Synthesis of 3-((4-(1-(2-oxo-2-(piperazin-J-yl)ethyl)piperidin-4-yl)phenyl)amino)piperidine-2,6-dione (057-5). The solution of tert-butyl 4-(2-(4-(4-((2,6-dioxopiperidin-3-yl)amino)phenyl)piperidin-1-yl)acetyl)piperazine-1-carboxylate (057-4) (200 mg, 389 μmol, 1.00 eq) in TFA (0.5 mL) and DCM (1.5 mL) was stirred at 25 °C for 2 h. LCMS showed tert-butyl 4-(2-(4-(4-((2,6-dioxopiperidin-3-yl)amino)phenyl)piperidin-1-yl)acetyl)piperazine-1-carboxylate (057-4) was consumed and the desired mass was detected. The mixture was concentrated in vacuo to give the title compound (057-5) (210 mg, crude, TFA) as a white solid. LCMS (ES+): m/z 414.3 [M+H]+.

Synthesis of cis-3-((4-(1-(2-(4-(3-((4-([1,1′-biphenyl]-3-yl)-5-chloropyrimidin-2-yl)amino)cyclohexane-1-carbonyl)piperazin-1-yl)-2-oxoethyl)piperidin-4-yl)phenyl)amino)piperidine-2,6-dione (Compound 57). To a solution of cis-3-((4-([1,1′-biphenyl]-3-yl)-5-chloropyrimidin-2-yl)amino)cyclohexane-1-carboxylic acid (168 mg, 379 mol, 1.00 eq, HCl) in DMF (2 mL) were added HATU (216 mg, 569 μmol, 1.50 eq), DIEA (147 mg, 1.14 mmol, 198 μL, 3.00 eq) and 3-((4-(1-(2-oxo-2-(piperazin-1-yl)ethyl)piperidin-4-yl)phenyl)amino)piperidine-2,6-dione (057-5) (200 mg, 379 μmol, 1.00 eq, TFA) at 25 °C. The mixture was stirred at 25 °C for 1 h. LCMS showed 3-((4-(1-(2-oxo-2-(piperazin-1-yl)ethyl)piperidin-4-yl)phenyl)amino)piperidine-2,6-dione (057-5) was consumed and the desired mass was detected. The mixture was diluted with H₂O (10 mL) and then extracted with EtOAc (5 mL*3). The combined organic layers were dried over Na₂SO₄, filtered and the filtrate was concentrated in vacuo. The residue was purified by prep-HPLC (column: Phenomenex luna C18 80*40 mm*3 μm; mobile phase: [water (FA)-ACN]; B %: 30%-70%, 8 min) to give the title compound (Compound 57) (80.5 mg, 98.8 μmol, 26.1% yield, 98.6% purity) as a white solid. LCMS (ES+): m/z 803.2 [M+H]+.

### Example 58. Synthesis of cis-3-(5-(1-(2-(4-(3-((4-([1,1′-biphenyl]-3-yl)-5-chloropyrimidin-2-yl)amino)cyclohexane-1-carbonyl)piperazin-1-yl)acetyl)piperidin-4-yl)-3-methyl-2-oxo-2,3-dihydro-1H-benzo[d]imidazol-1-yl)piperidine-2,6-dione (Compound 58)

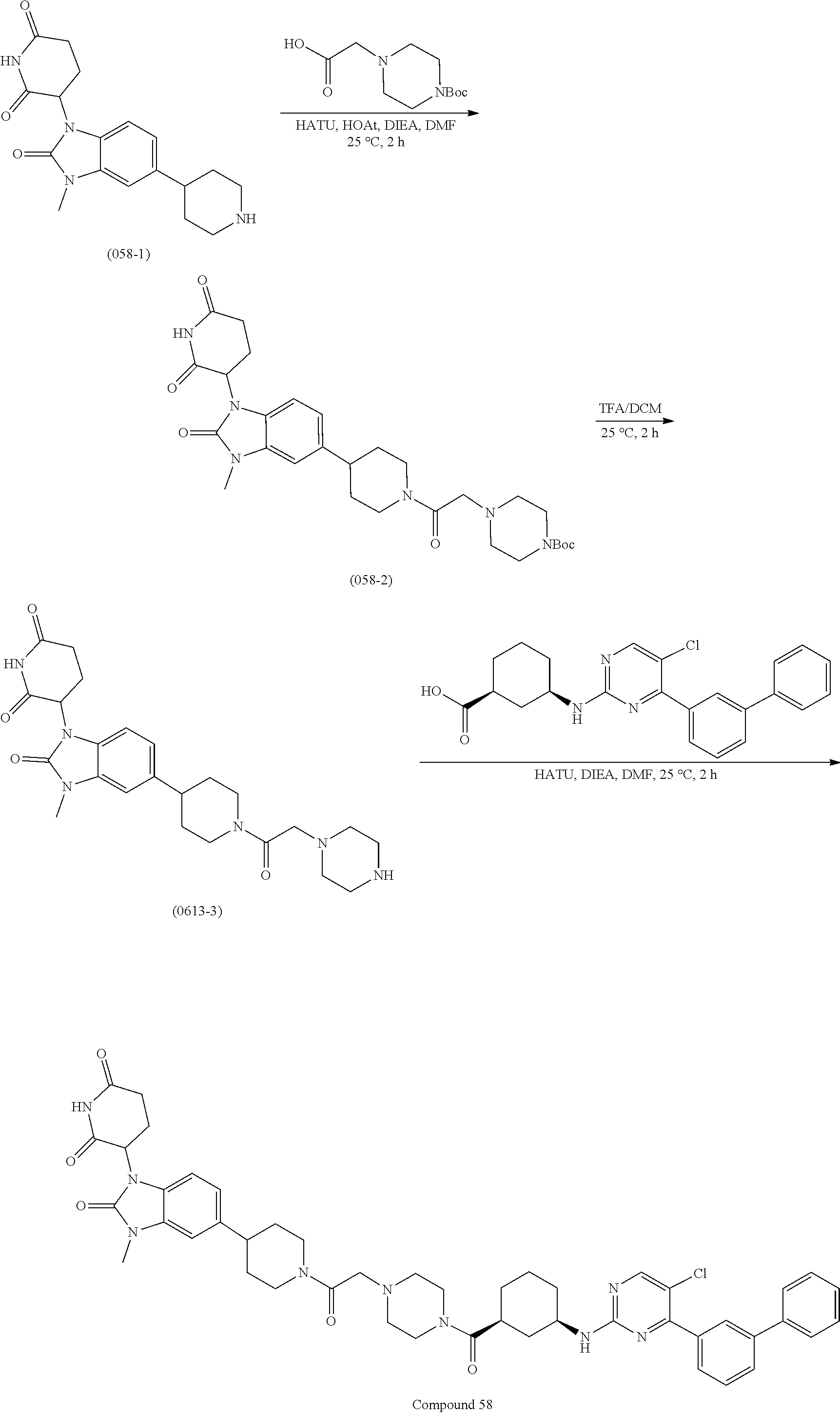 [SMILES: Cn1c(=O)n(C2CCC(=O)NC2=O)c2ccc(C3CCNCC3)cc21 ; CC(C)(C)OC(=O)N1CCN(CC(=O)O)CC1 ; Cn1c(=O)n(C2CCC(=O)NC2=O)c2ccc(C3CCN(C(=O)CN4CCN(C(=O)OC(C)(C)C)CC4)CC3)cc21 ; Cn1c(=O)n(C2CCC(=O)NC2=O)c2ccc(C3CCN(C(=O)CN4CCNCC4)CC3)cc21 ; O=C(O)[C@H]1CCC[C@@H](Nc2ncc(Cl)c(-c3cccc(-c4ccccc4)c3)n2)C1 ; Cn1c(=O)n(C2CCC(=O)NC2=O)c2ccc(C3CCN(C(=O)CN4CCN(C(=O)[C@H]5CCC[C@@H](Nc6ncc(Cl)c(-c7cccc(-c8ccccc8)c7)n6)C5)CC4)CC3)cc21]

Synthesis of tert-butyl 4-(2-(4-(1-(2,6-dioxopiperidin-3-yl)-3-methyl-2-oxo-2,3-dihydro-1H-benzo[d]imidazol-5-yl)piperidin-1-yl)-2-oxoethyl)piperazine-1-carboxylate (058-2). To a stirred solution of 2-(4-(tert-butoxycarbonyl)piperazin-1-yl)acetic acid (109 mg, 447 mol, 1.20 eq) in DMF (2 mL) were added HATU (212 mg, 559 μmol, 1.50 eq), HOAt (25.4 mg, 186 μmol, 26.1 μL, 0.500 eq) and DIEA (96.3 mg, 745 μmol, 130 μL, 2.00 eq), then to the mixture was added 3-(3-methyl-2-oxo-5-(piperidin-4-yl)-2,3-dihydro-1H-benzo[d]imidazol-1-yl)piperidine-2,6-dione (058-1) (170 mg, 372 μmol, 1.00 eq, TFA) at 25 °C. The mixture was stirred at 25 °C for 2 h. LCMS showed 3-(3-methyl-2-oxo-5-(piperidin-4-yl)-2,3-dihydro-1H-benzo[d]imidazol-1-yl)piperidine-2,6-dione (058-1) remained partly and the desired mass was detected. The mixture was diluted with H₂O (3 mL) and then extracted with EtOAc (3 mL*3). The combined organic layers were washed with saturated aqueous NaCl solution (5 mL*3), dried over Na₂SO₄, filtered and the filtrate was concentrated in vacuo. The mixture was purified by prep-TLC (SiO₂, Dichloromethane Methanol=10:1) to give the title compound (058-2) (185 mg, 325 μmol, 87.3% yield) as a yellow oil. LCMS (ES+): m/z 569.3 [M+H]+.

Synthesis of 3-(3-methyl-2-oxo-5-(1-(2-(piperazin-1-yl)acetyl)piperidin-4-yl)-2,3-dihydro-1H-benzo[d]imidazol-1-yl)piperidine-2,6-dione (058-3). A solution of tert-butyl 4-(2-(4-(1-(2,6-dioxopiperidin-3-yl)-3-methyl-2-oxo-2,3-dihydro-1H-benzo[d]imidazol-5-yl)piperidin-1-yl)-2-oxoethyl)piperazine-1-carboxylate (058-2) (185 mg, 325 μmol, 1.00 eq) in TFA (1 mL) and DCM (5 mL) was stirred at 25 °C for 2 h. LCMS showed tert-butyl 4-(2-(4-(1-(2,6-dioxopiperidin-3-yl)-3-methyl-2-oxo-2,3-dihydro-1H-benzo[d]imidazol-5-yl)piperidin-1-yl)-2-oxoethyl)piperazine-1-carboxylate (058-2) was consumed and the desired mass was detected. The mixture was concentrated in vacuo to give the title compound (058-3) (189 mg, crude, TFA) as a yellow oil. LCMS (ES+): m/z 469.2 [M+H]+.

Synthesis of cis-3-(5-(1-(2-(4-(3-((4-([1,1′-biphenyl]-3-yl)-5-chloropyrimidin-2-yl)amino)cyclohexane-1-carbonyl)piperazin-1-yl)acetyl)piperidin-4-yl)-3-methyl-2-oxo-2,3-dihydro-1H-benzo[d]imidazol-1-yl)piperidine-2,6-dione (Compound 58). To a solution of cis-3-((4-([1,1′-biphenyl]-3-yl)-5-chloropyrimidin-2-yl)amino)cyclohexane-1-carboxylic acid (173 mg, 389 μmol, 1.20 eq) (prepared by methods analogous to those shown in Example 22) in DMF (2 mL) were added HATU (185 mg, 487 μmol, 1.50 eq) and DIEA (210 mg, 1.62 mmol, 283 μL, 5.00 eq) at 25 °C. Then 3-(3-methyl-2-oxo-5-(1-(2-(piperazin-1-yl)acetyl)piperidin-4-yl)-2,3-dihydro-1H-benzo[d]imidazol-1-yl)piperidine-2,6-dione (058-3) (189 mg, 324 μmol, 1.00 eq, TFA) was added to the mixture at 25 °C. The resulting mixture was stirred at 25 °C for 2 h. LCMS showed 3-(3-methyl-2-oxo-5-(1-(2-(piperazin-1-yl)acetyl)piperidin-4-yl)-2,3-dihydro-1H-benzo[d]imidazol-1-yl)piperidine-2,6-dione (058-3) was consumed and the desired mass was detected. The mixture was diluted with H₂O (3 mL) and then extracted with EtOAc (3 mL*3). The combined organic layers were washed with saturated aqueous NaCl solution (5 mL*3), dried over Na₂SO₄, filtered and the filtrate was concentrated in vacuo. The mixture was purified by prep-HPLC (column: Phenomenex Luna C18 200*40 mm*10 μm; mobile phase: [water (FA)-ACN]; B %: 45%-75%, 8 min) to give the title compound (Compound 58) (40.0 mg, 44.3 μmol, 13.7% yield, 95.1% purity) as a white solid. LCMS (ES+): m/z 858.2 [M+H]+.

### Example 59. Synthesis of cis-3-(5-(1-(2-(1-(3-((4-([1,1′-biphenyl]-3-yl)-5-chloropyrimidin-2-yl)amino)cyclohexane-1-carbonyl)piperidin-4-yl)acetyl)piperidin-4-yl)-3-methyl-2-oxo-2,3-dihydro-1H-benzo[d]imidazol-1-yl)piperidine-2,6-dione (Compound 59)

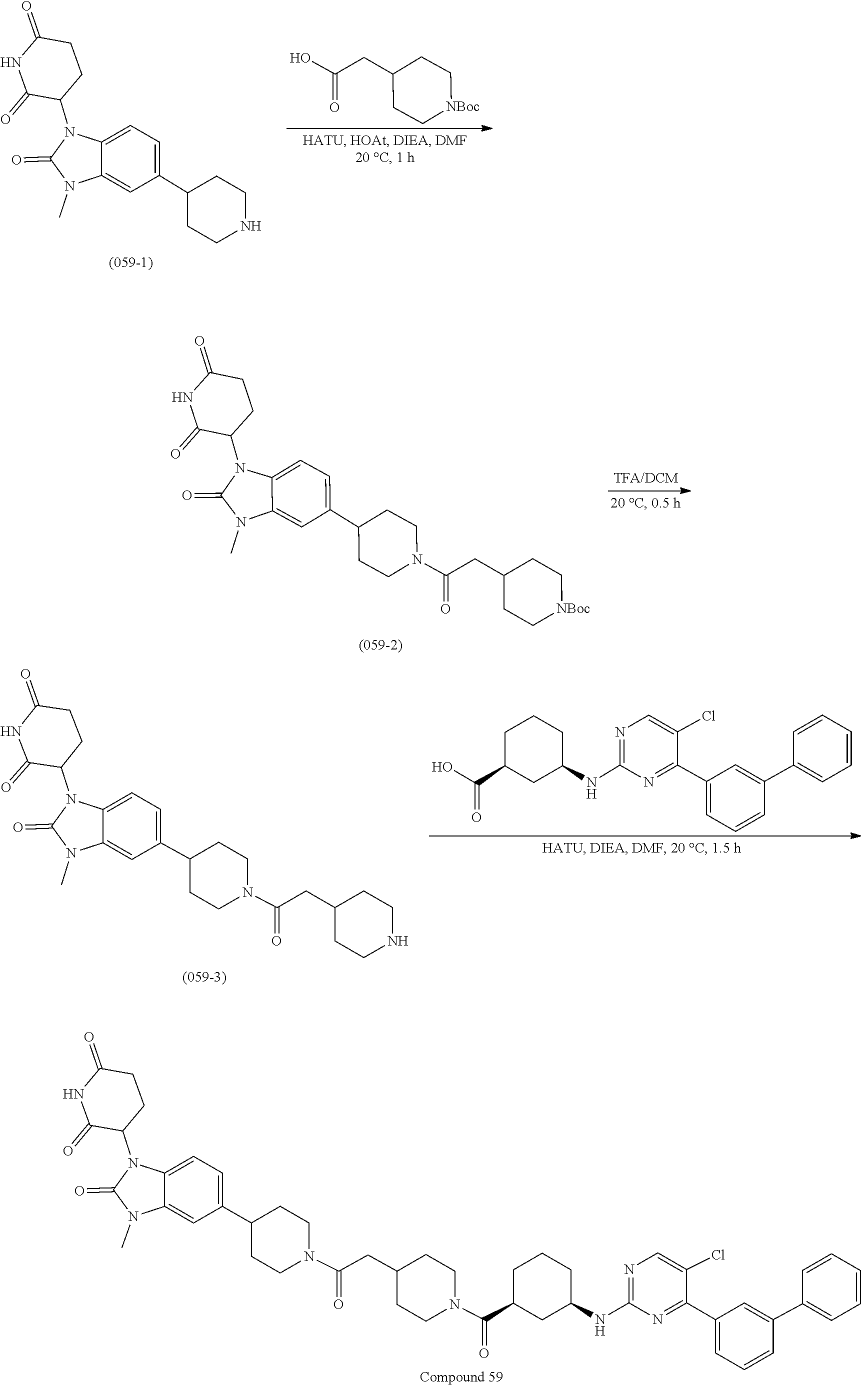 [SMILES: Cn1c(=O)n(C2CCC(=O)NC2=O)c2ccc(C3CCNCC3)cc21 ; CC(C)(C)OC(=O)N1CCC(CC(=O)O)CC1 ; Cn1c(=O)n(C2CCC(=O)NC2=O)c2ccc(C3CCN(C(=O)CC4CCN(C(=O)OC(C)(C)C)CC4)CC3)cc21 ; Cn1c(=O)n(C2CCC(=O)NC2=O)c2ccc(C3CCN(C(=O)CC4CCNCC4)CC3)cc21 ; O=C(O)[C@H]1CCC[C@@H](Nc2ncc(Cl)c(-c3cccc(-c4ccccc4)c3)n2)C1 ; Cn1c(=O)n(C2CCC(=O)NC2=O)c2ccc(C3CCN(C(=O)CC4CCN(C(=O)[C@H]5CCC[C@@H](Nc6ncc(Cl)c(-c7cccc(-c8ccccc8)c7)n6)C5)CC4)CC3)cc21]

Synthesis of tert-butyl 4-(2-(4-(1-(2,6-dioxopiperidin-3-yl)-3-methyl-2-oxo-2,3-dihydro-1H-benzo[d]imidazol-5-yl)piperidin-1-yl)-2-oxoethyl)piperidine-1-carboxylate (059-2). To a solution of 2-(1-(tert-butoxycarbonyl)piperidin-4-yl)acetic acid (109 mg, 447 μmol, 1.20 eq) in DMF (2 mL) were added HATU (212 mg, 559 μmol, 1.50 eq), HOAt (25.4 mg, 186 μmol, 26.1 μL, 0.500 eq) and DIEA (96.3 mg, 745 μmol, 130 μL, 2.00 eq) at 20 °C. Then to the solution was added 3-(3-methyl-2-oxo-5-(piperidin-4-yl)-2,3-dihydro-1H-benzo[d]imidazol-1-yl)piperidine-2,6-dione (059-1) (170 mg, 372 μmol, 1.00 eq, TFA) at 20 °C. The mixture was stirred at 20 °C for 1 h. LCMS showed 3-(3-methyl-2-oxo-5-(piperidin-4-yl)-2,3-dihydro-1H-benzo[d]imidazol-1-yl)piperidine-2,6-dione (059-1) was consumed and the desired mass was detected. The mixture was added H₂O (10 mL) and the mixture was extracted with EtOAc (10 mL*3), the combined organic layers were dried over Na₂SO₄, filtered and the filtrate was concentrated in vacuo. The mixture was purified by prep-TLC (SiO₂, EtOAc:MeOH=3:1) to give the title compound (059-2) (250 mg, crude) as a yellow oil. LCMS (ES+): m/z 568.3 [M+H]+.

Synthesis of 3-(3-methyl-2-oxo-5-(1-(2-(piperidin-4-yl)acetyl)piperidin-4-yl)-2,3-dihydro-1H-benzo[d]imidazol-1-yl)piperidine-2,6-dione (059-3). The solution of tert-butyl 4-(2-(4-(1-(2,6-dioxopiperidin-3-yl)-3-methyl-2-oxo-2,3-dihydro-1H-benzo[d]imidazol-5-yl)piperidin-1-yl)-2-oxoethyl)piperidine-1-carboxylate (059-2) (250 mg, 440 μmol, 1.00 eq) in TFA (1 mL) and DCM (5 mL) was stirred at 20 °C for 0.5 h. LCMS showed tert-butyl 4-(2-(4-(1-(2,6-dioxopiperidin-3-yl)-3-methyl-2-oxo-2,3-dihydro-1H-benzo[d]imidazol-5-yl)piperidin-1-yl)-2-oxoethyl)piperidine-1-carboxylate (059-2) was consumed and the desired mass was detected. The mixture was concentrated in vacuo to give the title compound (059-3) (256 mg, crude, TFA) as a yellow oil. LCMS (ES+): m/z 468.3 [M+H]+.

Synthesis of cis-3-(5-(1-(2-(1-(3-((4-([1,1′-biphenyl]-3-yl)-5-chloropyrimidin-2-yl)amino)cyclohexane-1-carbonyl)piperidin-4-yl)acetyl)piperidin-4-yl)-3-methyl-2-oxo-2,3-dihydro-1H-benzo[d]imidazol-1-yl)piperidine-2,6-dione (Compound 59). To a solution of cis-3-((4-([1,1′-biphenyl]-3-yl)-5-chloropyrimidin-2-yl)amino)cyclohexane-1-carboxylic acid (235 mg, 528 μmol, 1.20 eq, HCl) (prepared by methods analogous to those shown in Example 22) in DMF (2 mL) were added HATU (251 mg, 660 μmol, 1.50 eq), DIEA (284 mg, 2.20 mmol, 383 μL, 5.00 eq) and 3-(3-methyl-2-oxo-5-(1-(2-(piperidin-4-yl)acetyl)piperidin-4-yl)-2,3-dihydro-1H-benzo[d]imidazol-1-yl)piperidine-2,6-dione (059-3) (256 mg, 440 μmol, 1.00 eq, TFA) at 20 °C. The mixture was stirred at 20 °C for 1.5 h. LCMS showed 3-(3-methyl-2-oxo-5-(1-(2-(piperidin-4-yl)acetyl)piperidin-4-yl)-2,3-dihydro-1H-benzo[d]imidazol-1-yl)piperidine-2,6-dione (059-3) was consumed and the desired mass was detected. To the mixture was added H₂O (10 mL) and the mixture was extracted with EtOAc (10 mL*3), the combined organic layers were dried over Na₂SO₄, filtered and the filtrate was concentrated in vacuo. The mixture was purified by prep-HPLC (column: Phenomenex Luna C18 200*40 mm*10 μm; mobile phase: [water (FA)-ACN]; B %: 60%-90%, 8 min) to give the title compound (Compound 59) (111.2 mg, 128 μmol, 29.0% yield, 98.6% purity) as a yellow solid. LCMS (ES+): m/z 857.4 [M+H]+.

### Example 60. Synthesis of cis-3-(5-(1-(1-(3-((4-([1,1′-biphenyl]-3-yl)-5-chloropyrimidin-2-yl)amino)cyclohexane-1-carbonyl)piperidine-4-carbonyl)piperidin-4-yl)-1-oxoisoindolin-2-yl)piperidine-2,6-dione (Compound 60)

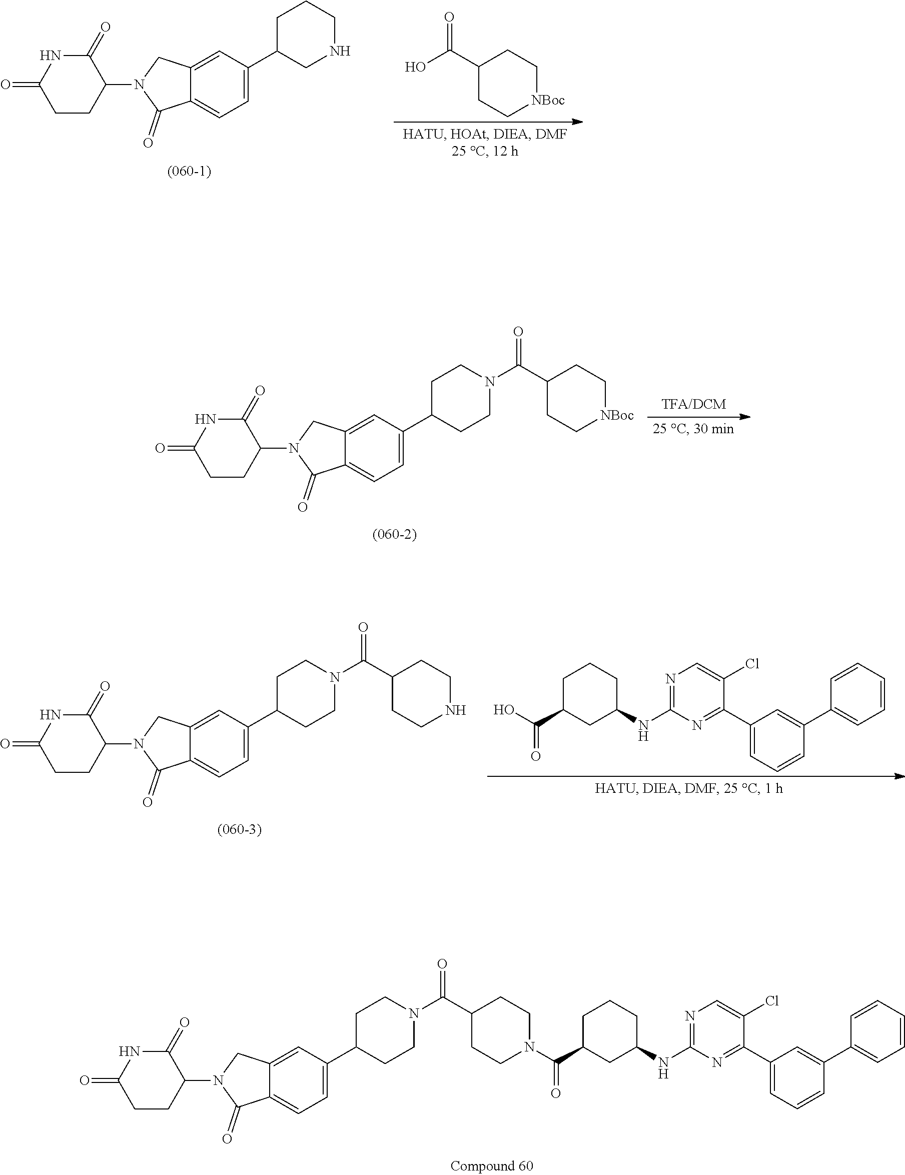 [SMILES: O=C1CCC(N2Cc3cc(C4CCCNC4)ccc3C2=O)C(=O)N1 ; CC(C)(C)OC(=O)N1CCC(C(=O)O)CC1 ; CC(C)(C)OC(=O)N1CCC(C(=O)N2CCC(c3ccc4c(c3)CN(C3CCC(=O)NC3=O)C4=O)CC2)CC1 ; O=C1CCC(N2Cc3cc(C4CCN(C(=O)C5CCNCC5)CC4)ccc3C2=O)C(=O)N1 ; O=C(O)[C@H]1CCC[C@@H](Nc2ncc(Cl)c(-c3cccc(-c4ccccc4)c3)n2)C1 ; O=C1CCC(N2Cc3cc(C4CCN(C(=O)C5CCN(C(=O)[C@H]6CCC[C@@H](Nc7ncc(Cl)c(-c8cccc(-c9ccccc9)c8)n7)C6)CC5)CC4)ccc3C2=O)C(=O)N1]

Synthesis of tert-butyl 4-(4-(2-(2,6-dioxopiperidin-3-yl)-1-oxoisoindolin-5-yl)piperidine-1-carbonyl)piperidine-1-carboxylate (060-2). To a solution of 3-(1-oxo-5-(piperidin-4-yl)isoindolin-2-yl)piperidine-2,6-dione (060-1) (300 mg, 677 μmol, 1.00 eq, TFA) in DMF (6 mL) were added 1-(tert-butoxycarbonyl)piperidine-4-carboxylic acid (187 mg, 816 μmol, 1.20 eq), DIEA (440 mg, 3.40 mmol, 592 μL, 5.00 eq), HOAt (46.3 mg, 340 μmol, 47.5 μL, 0.500 eq) and HATU (388 mg, 1.02 mmol, 1.50 eq) at 25 °C. The mixture was stirred at 25 °C for 12 h. LCMS showed 3-(1-oxo-5-(piperidin-4-yl)isoindolin-2-yl)piperidine-2,6-dione (060-1) was consumed completely and the desired mass was detected. Water (10 mL) was added to reaction, the aqueous phase was extracted with EtOAc (5 mL*3), the combined organic layers were dried over Na₂SO₄, filtered and the filtrate was concentrated under reduced pressure. The residue was purified by prep-TLC (SiO₂, Ethyl acetate:MeOH=20:1) to give the title compound (060-2) (365 mg, 678 μmol, 99.7% yield) as a brown solid. LCMS (ES+): m/z 539.2 [M+H]+.

Synthesis of 3-(1-oxo-5-(1-(piperidine-4-carbonyl)piperidin-4-yl)isoindolin-2-yl)piperidine-2,6-dione (060-3). A solution of tert-butyl 4-(4-(2-(2,6-dioxopiperidin-3-yl)-1-oxoisoindolin-5-yl)piperidine-1-carbonyl)piperidine-1-carboxylate (060-2) (365 mg, 678 mol, 1.00 eq) in TFA (1 mL) and DCM (5 mL) was stirred at 25 °C for 0.5 h. LCMS showed tert-butyl 4-(4-(2-(2,6-dioxopiperidin-3-yl)-1-oxoisoindolin-5-yl)piperidine-1-carbonyl)piperidine-1-carboxylate (060-2) was consumed completely and the desired mass was detected. The reaction mixture was concentrated under reduced pressure to give the title compound (060-3) (374 mg, crude, TFA) as a yellow oil. LCMS (ES+): m/z 439.2 [M+H]+.

Synthesis of cis-3-(5-(1-(1-(3-((4-([1,1′-biphenyl]-3-yl)-5-chloropyrimidin-2-yl)amino)cyclohexane-1-carbonyl)piperidine-4-carbonyl)piperidin-4-yl)-1-oxoisoindolin-2-yl)piperidine-2,6-dione (Compound 60). To a solution of 3-(1-oxo-5-(1-(piperidine-4-carbonyl)piperidin-4-yl)isoindolin-2-yl)piperidine-2,6-dione (060-3) (374 mg, 677 μmol, 1.00 eq, TFA) in DMF (8 mL) were added cis-3-((4-([1,1′-biphenyl]-3-yl)-5-chloropyrimidin-2-yl)amino)cyclohexane-1-carboxylic acid (361 mg, 812 μmol, 1.20 eq, HCl), DIEA (437 mg, 3.38 mmol, 589 μL, 5.00 eq) and HATU (386 mg, 1.02 mmol, 1.50 eq) at 25 °C. The mixture was stirred at 25 °C for 1 h. LCMS showed 3-(1-oxo-5-(1-(piperidine-4-carbonyl)piperidin-4-yl)isoindolin-2-yl)piperidine-2,6-dione (060-3) was consumed completely and the desired mass was detected. Water (10 mL) was added to reaction, the aqueous phase was extracted with EtOAc (10 mL*3), the combined organic layers were dried over Na₂SO₄, filtered and the filtrate was concentrated under reduced pressure. The residue was purified by prep-HPLC (column: Phenomenex C18 75*30 mm*3 μm; mobile phase: [water (FA)-ACN]; B %: 45%-70%, 8 min) to give the title compound (Compound 60) (60.1 mg, 71.8 μmol, 10.6% yield, 98.9% purity) as a white solid. LCMS (ES+): m/z 828.2 [M+H]+.

### Example 61. Synthesis of cis-3-(5-(1-(2-(4-(3-((4-([1,1′-biphenyl]-3-yl)-5-chloropyrimidin-2-yl)amino)cyclohexane-1-carbonyl)piperazin-1-yl)ethyl)piperidin-4-yl)-1-oxoisoindolin-2-yl)piperidine-2,6-dione (Compound 61)

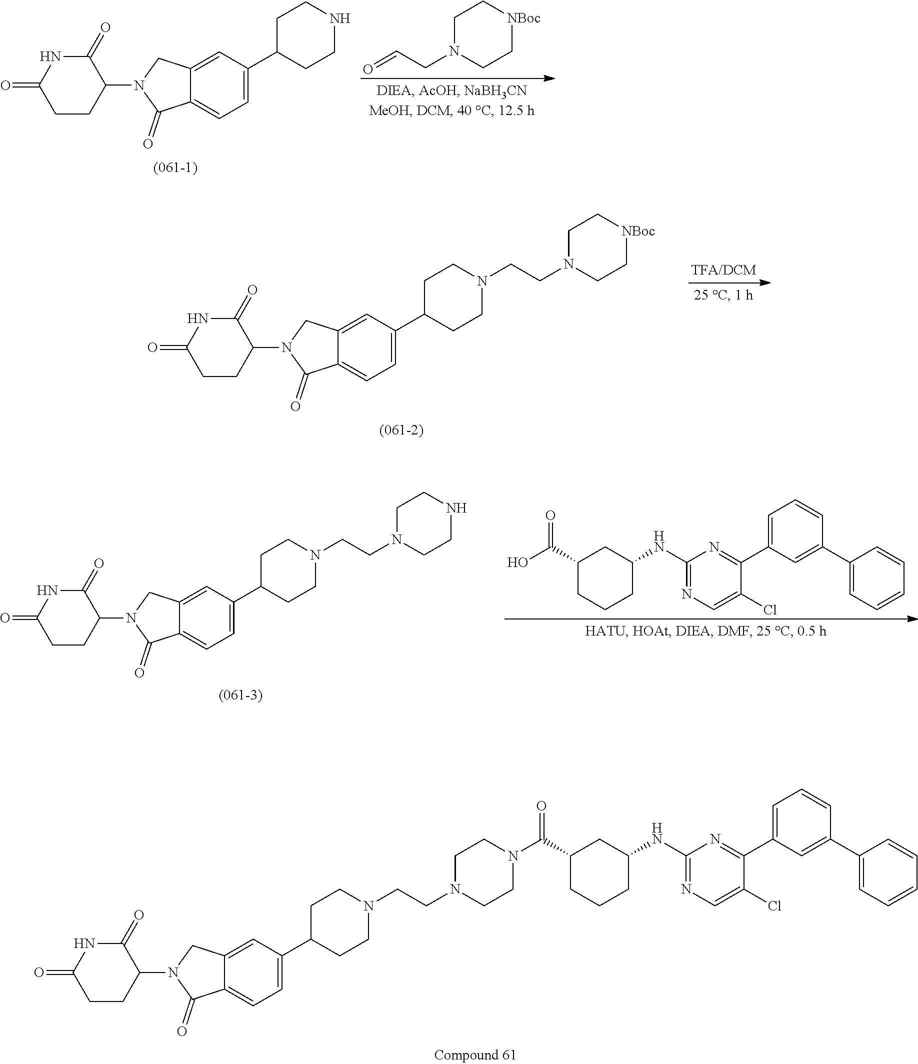

Synthesis of tert-butyl 4-(2-(4-(2-(2,6-dioxopiperidin-3-yl)-1-oxoisoindolin-5-yl)piperidin-1-yl)ethyl)piperazine-1-carboxylate (061-2). To a solution of 3-(1-oxo-5-(piperidin-4-yl)isoindolin-2-yl)piperidine-2,6-dione (061-1) (322 mg, 730 μmol, 1.00 eq, TFA) in DCM (4 mL) and MeOH (2 mL) were added DIEA (94.3 mg, 730 μmol, 127 μL, 1.00 eq) to adjust pH to 8 at 25 °C. The mixture was stirred at 25 °C for 0.25 h. To the reaction was added tert-butyl 4-(2-oxoethyl)piperazine-1-carboxylate (500 mg, 2.19 mmol, 3.00 eq), AcOH (4.38 mg, 73.0 μmol, 4.18 μL, 0.100 eq) was added to adjust pH to 5 at 25 °C. The mixture was stirred at 25 °C for 0.25 h. To the reaction mixture was added NaBH₃CN (138 mg, 2.19 mmol, 3.00 eq) at 25 °C and the resulting mixture was stirred at 40 °C for 12 h. LCMS showed 3-(1-oxo-5-(piperidin-4-yl)isoindolin-2-yl)piperidine-2,6-dione (061-1) was consumed, desired target MS was detected. The mixture was concentrated in vacuo, then H₂O (6 mL) was added to the mixture and the mixture was extracted with EtOAc (6 mL*3), the combined organic layers were washed with saturated aqueous NaCl solution (6 mL*3), dried over Na₂SO₄, filtered and the filtrate was concentrated in vacuo to give a reside, the mixture was purified by MPLC (SiO₂, Dichloromethane:Methanol=1:0 to 10:1) to give the title compound (061-2) (100 mg, 185 μmol, 25.4% yield) as a yellow oil. LCMS (ES+): m/z 540.2 [M+H]+.

Synthesis of 3-(1-oxo-5-(1-(2-(piperazin-1-yl)ethyl)piperidin-4-yl)isoindolin-2-yl)piperidine-2,6-dione (061-3). A solution of tert-butyl 4-(2-(4-(2-(2,6-dioxopiperidin-3-yl)-1-oxoisoindolin-5-yl)piperidin-1-yl)ethyl)piperazine-1-carboxylate (061-2) (100 mg, 185 μmol, 1.00 eq) in DCM (2 mL) and TFA (0.7 mL) was stirred at 25 °C for 1 h. LCMS showed tert-butyl 4-(2-(4-(2-(2,6-dioxopiperidin-3-yl)-1-oxoisoindolin-5-yl)piperidin-1-yl)ethyl)piperazine-1-carboxylate (061-2) was consumed, desired target MS was detected, then the mixture was concentrated in vacuo to give the title compound (061-3) (100 mg, crude, TFA) as a yellow oil. LCMS (ES+): m/z 440.1 [M+H]+.

Synthesis of cis-3-(5-(1-(2-(4-(3-((4-([1,1′-biphenyl]-3-yl)-5-chloropyrimidin-2-yl)amino)cyclohexane-1-carbonyl)piperazin-1-yl)ethyl)piperidin-4-yl)-1-oxoisoindolin-2-yl)piperidine-2,6-dione (Compound 61). To a solution of cis-3-((4-([1,1′-biphenyl]-3-yl)-5-chloropyrimidin-2-yl)amino)cyclohexane-1-carboxylic acid (68.2 mg, 154 μmol, 1.00 eq, HCl) (prepared by methods analogous to those shown in Example 22) in DMF (2 mL) were added HATU (87.6 mg, 230 μmol, 1.50 eq), DIEA (99.2 mg, 768 μmol, 134 μL, 5.00 eq) and HOAt (10.5 mg, 76.8 μmol, 10.7 μL, 0.500 eq) at 25 °C. Then 3-(1-oxo-5-(1-(2-(piperazin-1-yl)ethyl)piperidin-4-yl)isoindolin-2-yl)piperidine-2,6-dione (061-3) (85.0 mg, 154 μmol, 1.00 eq, TFA) was added to the mixture, the mixture was stirred at 25 °C for 0.5 h. LCMS showed 3-(1-oxo-5-(1-(2-(piperazin-1-yl)ethyl)piperidin-4-yl)isoindolin-2-yl)piperidine-2,6-dione (061-3) was consumed, desired target MS was detected. Then H₂O (2 mL) was added to the mixture and the mixture was extracted with EtOAc (2 mL*3), the combined organic layers were washed with saturated aqueous NaCl solution (2 mL*3), dried over Na₂SO₄, filtered and the filtrate was concentrated in vacuo to give a residue, the residue was purified by prep-HPLC (column: Phenomenex Luna C18 200*40 mm*10 μm; mobile phase: [water (FA)-ACN]; B %: 15%-55%, 8 min) to give the title compound (Compound 61) (18.0 mg, 20.9 μmol, 13.6% yield, 96.2% purity) as a white solid. LCMS (ES+): m/z 829.4 [M+H]+.

### Example 62. Synthesis of cis-3-(5-(1-((1-(3-((4-([1,1′-biphenyl]-3-yl)-5-chloropyrimidin-2-yl)amino)cyclohexane-1-carbonyl)piperidin-4-yl)methyl)piperidin-4-yl)-1-oxoisoindolin-2-yl)piperidine-2,6-dione (Compound 63)

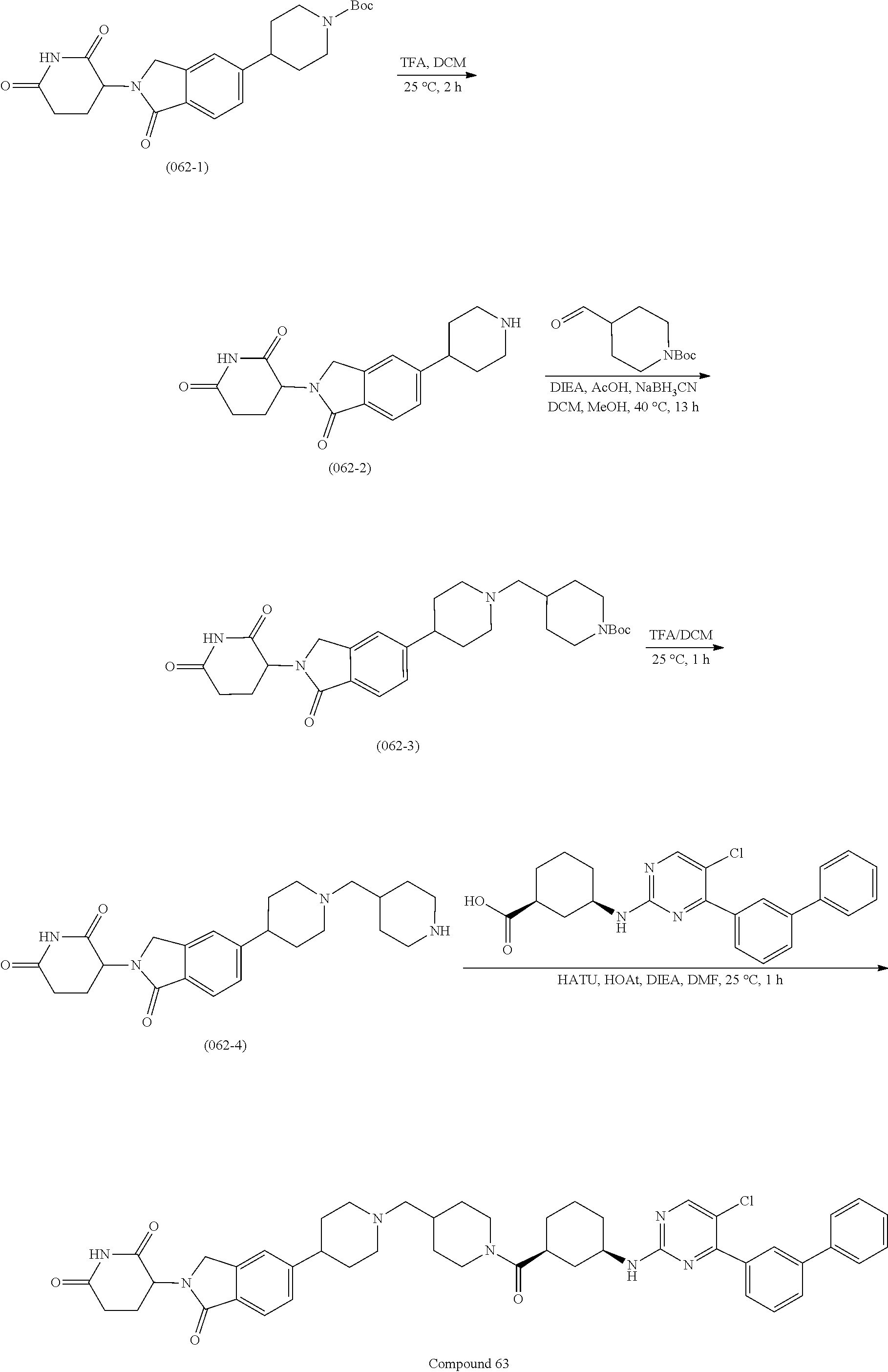

Synthesis of 3-(1-oxo-5-(piperidin-4-yl)isoindolin-2-yl)piperidine-2,6-dione. A solution of tert-butyl 4-(2-(2,6-dioxopiperidin-3-yl)-1-oxoisoindolin-5-yl)piperidine-1-carboxylate (5.00 g, 11.7 mmol, 1.00 eq) (prepared by methods known in the art) in TFA (12 mL) and DCM (60 mL) was stirred at 25 °C for 2 h. LCMS showed tert-butyl 4-(2-(2,6-dioxopiperidin-3-yl)-1-oxoisoindolin-5-yl)piperidine-1-carboxylate was consumed completely and the desired mass was detected. The reaction mixture was concentrated under reduced pressure to the title compound (5.00 g, crude, TFA) as a brown oil. LCMS (ES+): m/z 328.2 [M+H]+.

Synthesis of tert-butyl 4-((4-(2-(2,6-dioxopiperidin-3-yl)-1-oxoisoindolin-5-yl)piperidin-1-yl)methyl)piperidine-1-carboxylate. To a solution of 3-(1-oxo-5-(piperidin-4-yl)isoindolin-2-yl)piperidine-2,6-dione (300 mg, 680 μmol, 1.00 eq, TFA) in MeOH (2 mL) and DCM (1 mL) was added DIEA (264 mg, 2.04 mmol, 355 μL, 3.00 eq) to adjust pH to 8 at 25 °C. Tert-butyl 4-formylpiperidine-1-carboxylate (174 mg, 816 μmol, 1.20 eq) was added to the mixture at 25 °C. The mixture was stirred at 25 °C for 0.5 h. AcOH (40.8 mg, 680 μmol, 38.9 μL, 1.00 eq) was added dropwise until the pH=5 at 25 °C. The mixture was stirred at 25 °C for 0.5 h. NaBH₃CN (128 mg, 2.04 mmol, 3.00 eq) was added to the mixture at 25 °C. The mixture was stirred at 40 °C for 12 h. LCMS showed 3-(1-oxo-5-(piperidin-4-yl)isoindolin-2-yl)piperidine-2,6-dione was consumed mostly and the desired mass was detected. The reaction mixture was concentrated under reduced pressure to remove solvent. The residue was purified by prep-TLC (SiO₂, Dichloromethane:Methanol=20:1) to give the title compound (300 mg, crude) as a white solid. LCMS (ES+): m/z 525.2 [M+H]+.

Synthesis of 3-(1-oxo-5-(1-(piperidin-4-ylmethyl)piperidin-4-yl)isoindolin-2-yl)piperidine-2,6-dione. A solution of tert-butyl 4-((4-(2-(2,6-dioxopiperidin-3-yl)-1-oxoisoindolin-5-yl)piperidin-1-yl)methyl)piperidine-1-carboxylate (356 mg, 679 μmol, 1.00 eq) in DCM (5 mL) and TFA (1 mL) was stirred at 25 °C for 1 h. LCMS showed tert-butyl 4-((4-(2-(2,6-dioxopiperidin-3-yl)-1-oxoisoindolin-5-yl)piperidin-1-yl)methyl)piperidine-1-carboxylate was consumed completely and the desired mass was detected. The reaction mixture was concentrated under reduced pressure to give the title compound (365 mg, crude, TFA) as a yellow oil. LCMS (ES+): m/z 425.2 [M+H]+.

Synthesis of cis-3-(5-(1-((1-(3-((4-([1,1′-biphenyl]-3-yl)-5-chloropyrimidin-2-yl)amino)cyclohexane-1-carbonyl)piperidin-4-yl)methyl)piperidin-4-yl)-1-oxoisoindolin-2-yl)piperidine-2,6-dione (Compound 63). To a solution of 3-(1-oxo-5-(1-(piperidin-4-ylmethyl)piperidin-4-yl)isoindolin-2-yl)piperidine-2,6-dione (365 mg, 678 μmol, 1.00 eq, TFA) and cis-3-((4-([1,1′-biphenyl]-3-yl)-5-chloropyrimidin-2-yl)amino)cyclohexane-1-carboxylic acid (301 mg, 678 μmol, 1.00 eq, HCl) (prepared by methods analogous to those shown in Example 22) in DMF (4 mL) were added HATU (309 mg, 813 μmol, 1.20 eq), HOAt (46.1 mg, 339 μmol, 47.4 μL, 0.500 eq) and DIEA (438 mg, 3.39 mmol, 590 μL, 5.00 eq) at 25 °C. The mixture was stirred at 25 °C for 1 h. LCMS showed 3-(1-oxo-5-(1-(piperidin-4-ylmethyl)piperidin-4-yl)isoindolin-2-yl)piperidine-2,6-dione was consumed completely and the desired mass was detected. Water (10 mL) was added to the mixture at 25 °C. Then the reaction mixture was extracted with EtOAc (5 mL*3). The combined organic layers were dried over anhydrous sodium sulfate, filtered and the filtrate was concentrated under reduced pressure to give a residue. The residue was purified by prep-HPLC (column: Phenomenex Luna C18 75*30 mm*3 μm; mobile phase: [water (FA)-ACN]; B %: 30%-65%, 8 min) to give the title compound (Compound 63) (34.5 mg, 41.0 μmol, 6.04% yield, 96.7% purity) as a white solid. LCMS (ES+): m/z 814.3 [M+H]+.

### Example 63. Synthesis of cis-3-((4-(1-(2-(1-(3-((4-([1,1′-biphenyl]-3-yl)-5-chloropyrimidin-2-yl)amino)cyclohexane-1-carbonyl)piperidin-4-yl)ethyl)piperidin-4-yl)phenyl)amino)piperidine-2,6-dione (Compound 64)

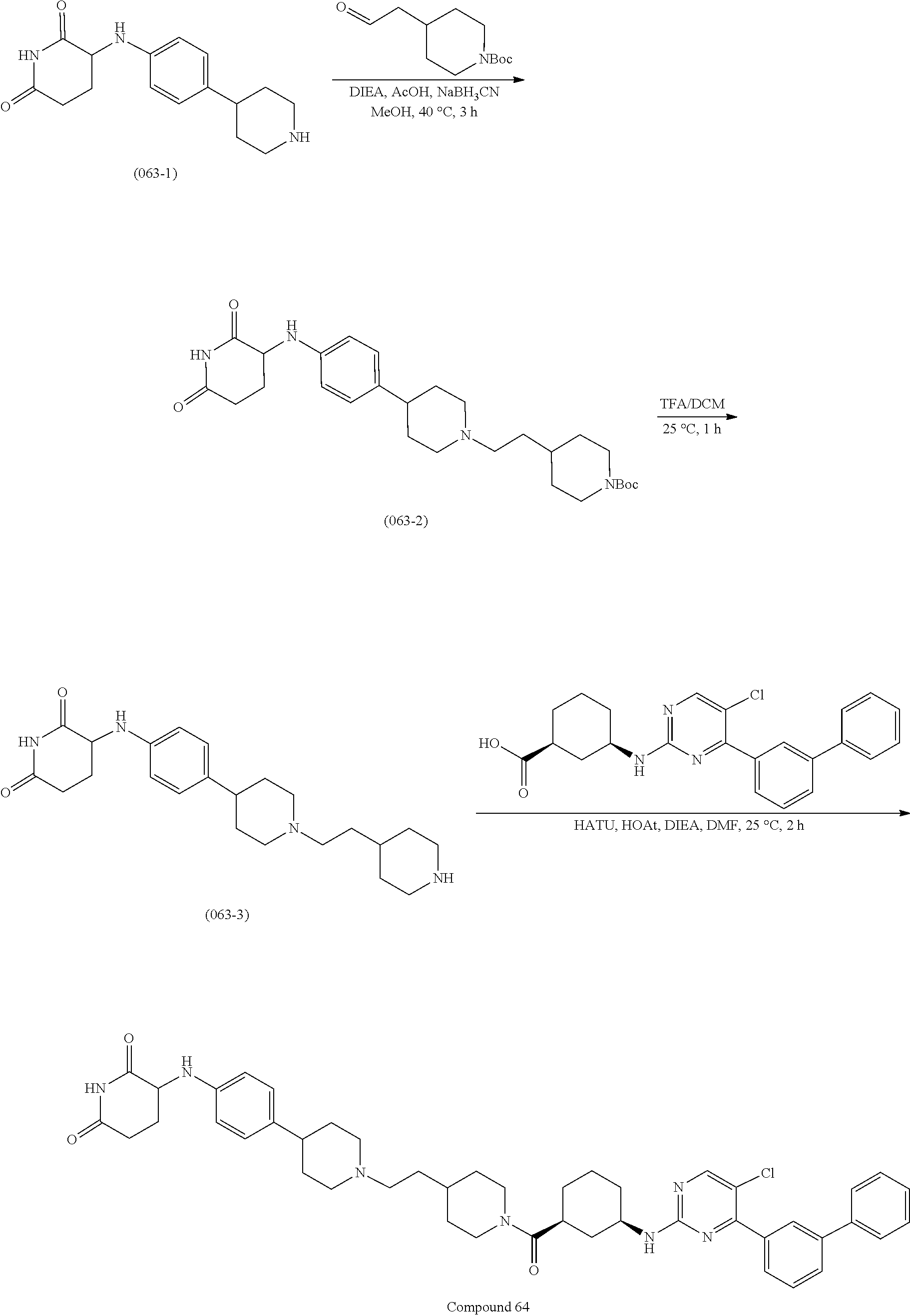 [SMILES: O=C1CCC(Nc2ccc(C3CCNCC3)cc2)C(=O)N1 ; CC(C)(C)OC(=O)N1CCC(CC=O)CC1 ; CC(C)(C)OC(=O)N1CCC(CCN2CCC(c3ccc(NC4CCC(=O)NC4=O)cc3)CC2)CC1 ; O=C1CCC(Nc2ccc(C3CCN(CCC4CCNCC4)CC3)cc2)C(=O)N1 ; O=C(O)[C@H]1CCC[C@@H](Nc2ncc(Cl)c(-c3cccc(-c4ccccc4)c3)n2)C1 ; O=C1CCC(Nc2ccc(C3CCN(CCC4CCN(C(=O)[C@H]5CCC[C@@H](Nc6ncc(Cl)c(-c7cccc(-c8ccccc8)c7)n6)C5)CC4)CC3)cc2)C(=O)N1]

Synthesis of tert-butyl 4-(2-(4-(4-((2,6-dioxopiperidin-3-yl)amino)phenyl)piperidin-1-yl)ethyl)piperidine-1-carboxylate (063-2). To a solution of 3-((4-(piperidin-4-yl)phenyl)amino)piperidine-2,6-dione (063-1) (200 mg, 498 μmol, 1.00 eq, TFA) and tert-butyl 4-(2-oxoethyl)piperidine-1-carboxylate (136 mg, 598 μmol, 1.20 eq) in MeOH (5 mL) was added DIEA until the pH=8. The mixture was stirred at 25 °C for 0.5 h. AcOH (29.9 mg, 498 μmol, 28.5 μL, 1.00 eq) was added dropwise until the pH=6 at 25 °C. The mixture was stirred at 25 °C for 0.5 h. NaBH₃CN (93.9 mg, 1.49 mmol, 3.00 eq) was added to the mixture at 25 °C. The mixture was stirred at 40 °C for 2 h. LCMS showed 3-((4-(piperidin-4-yl)phenyl)amino)piperidine-2,6-dione (063-1) was consumed completely and the desired mass was detected. The reaction mixture was concentrated under reduced pressure to remove solvent. The residue was purified by prep-TLC (SiO₂, Ethyl acetate:Methanol=20:1) to give the title compound (063-2) (640 mg, crude) as a yellow oil. LCMS (ES+): m/z 499.3 [M+H]+.

Synthesis of 3-((4-(1-(2-(piperidin-4-yl)ethyl)piperidin-4-yl)phenyl)amino)piperidine-2,6-dione (063-3). To a solution of tert-butyl 4-(2-(4-(4-((2,6-dioxopiperidin-3-yl)amino)phenyl)piperidin-1-yl)ethyl)piperidine-1-carboxylate (063-2) (200 mg, 401 μmol, 1.00 eq) in DCM (3 mL) was added TFA (1.54 g, 13.5 mmol, 1.00 mL, 33.7 eq) at 25 °C. The mixture was stirred at 25 °C for 1 h. LCMS showed tert-butyl 4-(2-(4-(4-((2,6-dioxopiperidin-3-yl)amino)phenyl)piperidin-1-yl)ethyl)piperidine-1-carboxylate (063-2) was consumed completely and the desired mass was detected. The reaction mixture was concentrated under reduced pressure to remove solvent to give the title compound (063-3) (200 mg, crude, TFA) as a yellow oil. LCMS (ES+): m/z 399.2 [M+H]+.

Synthesis of cis-3-((4-(1-(2-(1-(3-((4-([1,1′-biphenyl]-3-yl)-5-chloropyrimidin-2-yl)amino)cyclohexane-1-carbonyl)piperidin-4-yl)ethyl)piperidin-4-yl)phenyl)amino)piperidine-2,6-dione (Compound 64). To a solution of cis-3-((4-([1,1′-biphenyl]-3-yl)-5-chloropyrimidin-2-yl)amino)cyclohexane-1-carboxylic acid (173 mg, 390 mol, 1.00 eq) in DMF (4 mL) were added HATU (178 mg, 468 μmol, 1.20 eq), DIEA (252 mg, 1.95 mmol, 340 μL, 5.00 eq), HOAt (26.6 mg, 195 μmol, 27.3 μL, 0.500 eq) and 3-((4-(1-(2-(piperidin-4-yl)ethyl)piperidin-4-yl)phenyl)amino)piperidine-2,6-dione (063-3) (200 mg, 390 μmol, 1.00 eq, TFA) at 25 °C. The mixture was stirred at 25 °C for 2 h. LCMS showed 3-((4-(1-(2-(piperidin-4-yl)ethyl)piperidin-4-yl)phenyl)amino)piperidine-2,6-dione (063-3) was consumed completely and the desired mass was detected. Water (4 mL) was added to the mixture at 25 °C. Then the reaction mixture was extracted with EtOAc (3 mL*3). The organic layers were dried over anhydrous sodium sulfate, filtered and the filtrate was concentrated under reduced pressure to give a residue. The residue was purified by prep-HPLC (column: Phenomenex Luna C18 75*30 mm*3 μm; mobile phase: [water (FA)-ACN]; B %: 30%-70%, 8 min) to give the title compound (Compound 64) (60.5 mg, 74.5 μmol, 19.1% yield, 97.1% purity) as a white solid. LCMS (ES+): m/z 788.3 [M+H]+.

### Example 64. Synthesis of cis-3-((4-(1-((1-(3-((4-([1,1′-biphenyl]-3-yl)-5-chloropyrimidin-2-yl)amino)cyclohexane-1-carbonyl)piperidin-4-yl)methyl)piperidin-4-yl)phenyl)amino)piperidine-2,6-dione (Compound 65)

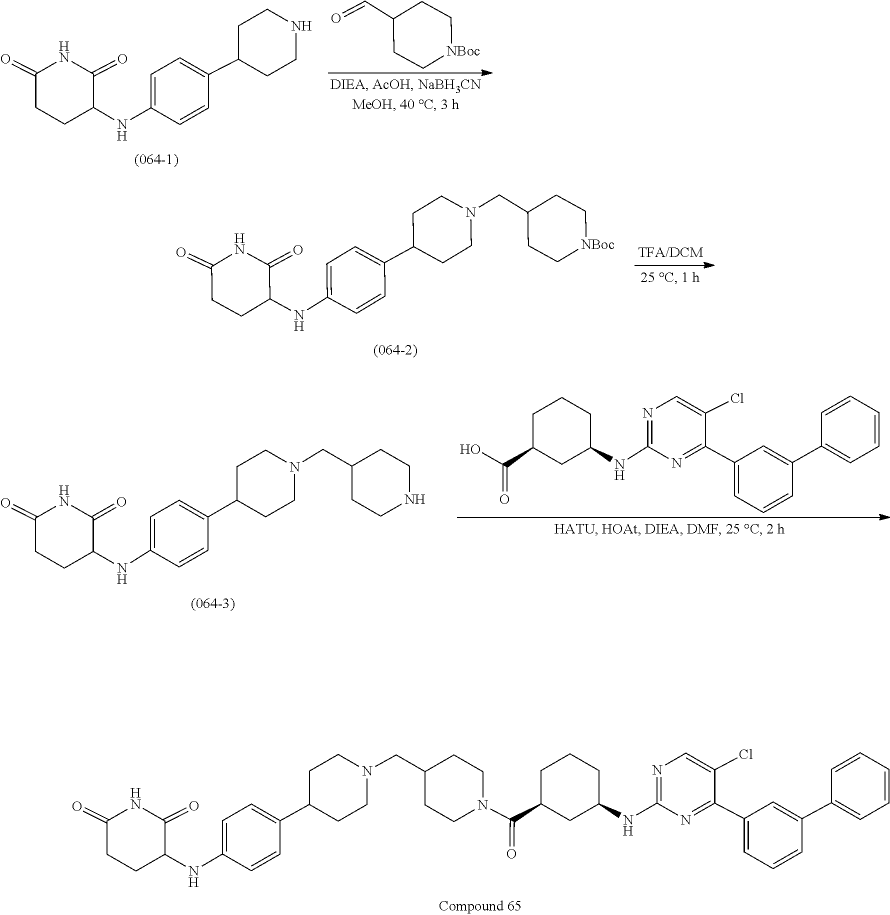

Synthesis of tert-butyl 4-((4-(4-((2,6-dioxopiperidin-3-yl)amino)phenyl)piperidin-1-yl)methyl)piperidine-1-carboxylate (064-2). To a solution of 3-((4-(piperidin-4-yl)phenyl)amino)piperidine-2,6-dione (064-1) (200 mg, 498 μmol, 1.00 eq, TFA) and tert-butyl 4-formylpiperidine-1-carboxylate (128 mg, 598 μmol, 1.20 eq) in MeOH (5 mL) was added DIEA dropwise until pH=8. The mixture was stirred at 25 °C for 0.5 h. AcOH (29.9 mg, 498 μmol, 28.5 μL, 1.00 eq) was added dropwise until the pH=6 at 25 °C. The mixture was stirred at 25 °C for 0.5 h. NaBH₃CN (93.9 mg, 1.49 mmol, 3.00 eq) was added to the mixture at 25 °C. The mixture was stirred at 40 °C for 2 h. LCMS showed ~25.6% of 3-((4-(piperidin-4-yl)phenyl)amino)piperidine-2,6-dione (064-1) was remaining and desired mass was detected. The reaction mixture was concentrated under reduced pressure to remove solvent. The residue was purified by prep-TLC (SiO₂, Ethyl acetate:Methanol=10:1) to give the title compound (064-2) (260 mg, crude) as a white solid. LCMS (ES+): m/z 485.2 [M+H]+.

Synthesis of 3-((4-(1-(piperidin-4-ylmethyl)piperidin-4-yl)phenyl)amino)piperidine-2,6-dione (064-3). To a solution of tert-butyl 4-((4-(4-((2,6-dioxopiperidin-3-yl)amino)phenyl)piperidin-1-yl)methyl)piperidine-1-carboxylate (064-2) (260 mg, 536 μmol, 1.00 eq) in DCM (3 mL) was added TFA (1.54 g, 13.5 mmol, 1.00 mL, 25.2 eq) at 25 °C. The mixture was stirred at 25 °C for 1 h. LCMS showed tert-butyl 4-((4-(4-((2,6-dioxopiperidin-3-yl)amino)phenyl)piperidin-1-yl)methyl)piperidine-1-carboxylate (064-2) was consumed completely and the desired mass was detected. The reaction mixture was concentrated under reduced pressure to remove solvent to give the title compound (064-3) (260 mg, crude, TFA) as a yellow oil. LCMS (ES+): m/z 385.1 [M+H]+.

Synthesis of cis-3-((4-(1-((1-(3-((4-([1,1′-biphenyl]-3-yl)-5-chloropyrimidin-2-yl)amino)cyclohexane-1-carbonyl)piperidin-4-yl)methyl)piperidin-4-yl)phenyl)amino)piperidine-2,6-dione (Compound 65). To a solution of cis-3-((4-([1,1′-biphenyl]-3-yl)-5-chloropyrimidin-2-yl)amino)cyclohexane-1-carboxylic acid (232 mg, 522 mol, 1.00 eq) (prepared by methods analogous to those shown in Example 22) in DMF (4 mL) were added HATU (238 mg, 626 μmol, 1.20 eq), DIEA (337 mg, 2.61 mmol, 454 μL, 5.00 eq), HOAt (35.5 mg, 261 μmol, 36.5 μL, 0.500 eq) and 3-((4-(1-(piperidin-4-ylmethyl)piperidin-4-yl)phenyl)amino)piperidine-2,6-dione (064-3) (260 mg, 522 μmol, 1.00 eq, TFA) at 25 °C. The mixture was stirred at 25 °C for 2 h. LCMS showed 3-((4-(1-(piperidin-4-ylmethyl)piperidin-4-yl)phenyl)amino)piperidine-2,6-dione (064-3) was consumed completely and the desired mass was detected. Water (4 mL) was added to the mixture at 25 °C. Then the reaction mixture was extracted with EtOAc (3 mL*3). The organic layers were dried over anhydrous sodium sulfate, filtered and the filtrate was concentrated under reduced pressure to give a residue. The residue was purified by prep-HPLC (column: Phenomenex Luna C18 75*30 mm*3 μm; mobile phase: [water (FA)-ACN]; B %: 30%-70%, 8 min) to give the title compound (Compound 65) (65.5 mg, 79.1 μmol, 15.2% yield, 93.5% purity) as a white solid. LCMS (ES+): m/z 774.2 [M+H]+.

### Example 65. Synthesis of N-(10-(4-((4-([1,1′-biphenyl]-3-yl)-5-chloropyrimidin-2-yl)amino)piperidin-1-yl)-10-oxodecyl)-2-((2-(2,6-dioxopiperidin-3-yl)-1,3-dioxoisoindolin-4-yl)oxy)acetamide (Compound 66)

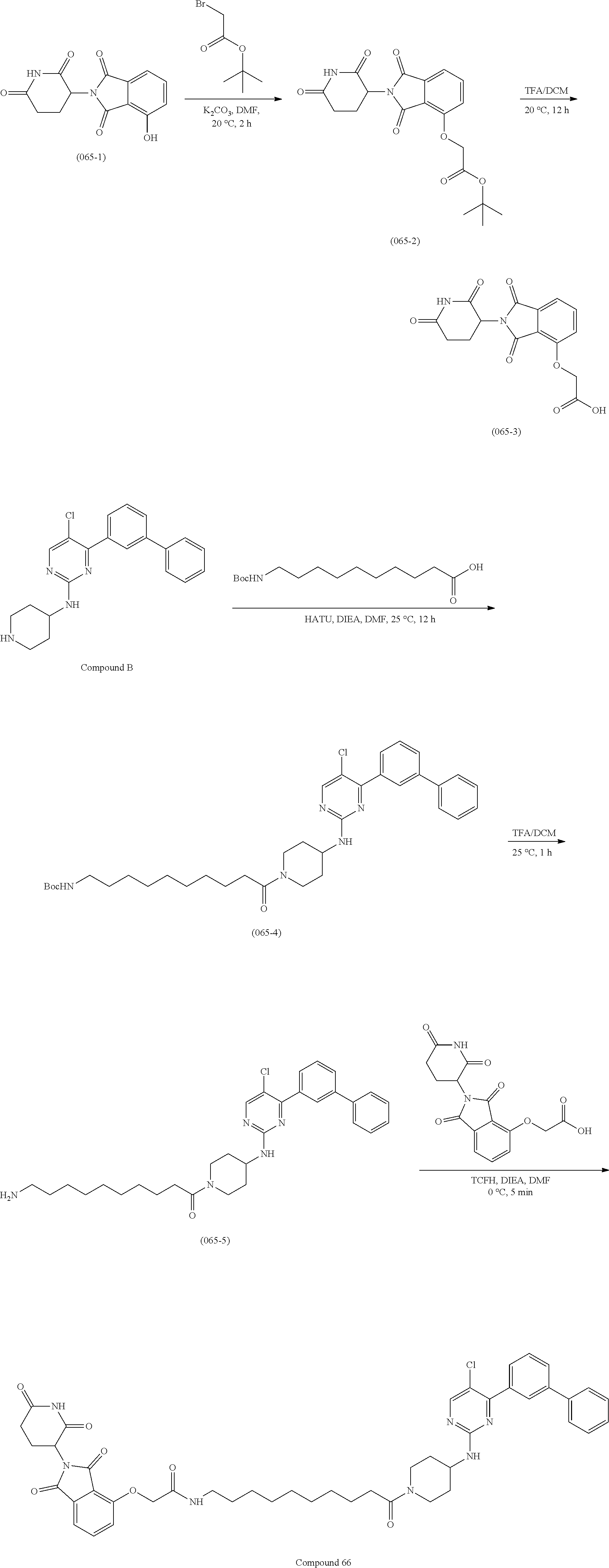

Synthesis of tert-butyl 2-((2-(2,6-dioxopiperidin-3-yl)-1,3-dioxoisoindolin-4-yl)oxy)acetate (065-2). To a solution of 2-(2,6-dioxopiperidin-3-yl)-4-hydroxyisoindoline-1,3-dione (065-1) (4.00 g, 14.6 mmol, 1.00 eq) in DMF (40 mL) were added K₂CO₃ (3.02 g, 21.9 mmol, 1.50 eq) and tert-butyl 2-bromoacetate (2.85 g, 14.6 mmol, 2.16 mL, 1.00 eq) at 20 °C. The mixture was stirred at 20 °C for 2 h. LCMS showed ~12.5% of 2-(2,6-dioxopiperidin-3-yl)-4-hydroxyisoindoline-1,3-dione (065-1) was remaining and desired mass was detected. Water (30 mL) was added to the mixture at 20 °C. Then the reaction mixture was extracted with EtOAc (20 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=1:0 to 1:3) to give the title compound (065-2) (5.40 g, 13.9 mmol, 95.3% yield) as a white solid. LCMS (ES⁺): m/z 411.1 [M+Na]⁺.

Synthesis of 2-((2-(2,6-dioxopiperidin-3-yl)-1,3-dioxoisoindolin-4-yl)oxy)acetic acid (065-3). To a solution of tert-butyl 2-((2-(2,6-dioxopiperidin-3-yl)-1,3-dioxoisoindolin-4-yl)oxy)acetate (065-2) (5.40 g, 13.9 mmol, 1.00 eq) in DCM (50 mL) was added TFA (15.4 g, 135 mmol, 10.0 mL, 9.71 eq) at 20 °C. The mixture was stirred at 20 °C for 12 h. LCMS showed ~3.3% of tert-butyl 2-((2-(2,6-dioxopiperidin-3-yl)-1,3-dioxoisoindolin-4-yl)oxy)acetate (065-2) was remaining and desired mass was detected. The reaction mixture was concentrated under reduced pressure to remove solvent to give the title compound (065-3) (4.60 g, crude) as a brown oil. LCMS (ES⁺): m/z 333.1 [M+H]⁺.

Synthesis of tert-butyl (10-(4-((4-([1,1′-biphenyl]-3-yl)-5-chloropyrimidin-2-yl)amino)piperidin-1-yl)-10-oxodecyl)carbamate (065-4). To a solution of 10-(tert-butoxycarbonylamino)decanoic acid (236 mg, 822 μmol, 1.00 eq) and 4-([1,1′-biphenyl]-3-yl)-5-chloro-N-(piperidin-4-yl)pyrimidin-2-amine (Compound B) (300 mg, 822 μmol, 1.00 eq) in DMF (3 mL) were added HATU (469 mg, 1.23 mmol, 1.50 eq) and DIEA (638 mg, 4.93 mmol, 859 μL, 6.00 eq) at 25 °C. The mixture was stirred at 25 °C for 12 h. LCMS showed 4-([1,1′-biphenyl]-3-yl)-5-chloro-N-(piperidin-4-yl)pyrimidin-2-amine (Compound B) was consumed completely and the desired mass was detected. Water (2 mL) was added to the mixture at 25 °C and extracted with EtOAc (1 mL*3). The organic layers were dried over anhydrous sodium sulfate, filtered and the filtrate was concentrated under reduced pressure to give a residue. The residue was purified by prep-TLC (SiO₂, Petroleum ether:Ethyl acetate=1:1) to give the title compound (065-4) (358 mg, 396 μmol, 48.2% yield, 70.1% purity) as a yellow oil. LCMS (ES+): m/z 634.4 [M+H]+.

Synthesis of 1-(4-((4-([1,1′-biphenyl]-3-yl)-5-chloropyrimidin-2-yl)amino)piperidin-1-yl)-10-aminodecan-1-one (065-5). To a solution of tert-butyl (10-(4-((4-([1,1′-biphenyl]-3-yl)-5-chloropyrimidin-2-yl)amino)piperidin-1-yl)-10-oxodecyl)carbamate (200 mg, 315 μmol, 1.00 eq) in DCM (3 mL) was added TFA (1.54 g, 13.5 mmol, 1.00 mL, 42.8 eq) at 25 °C. The mixture was stirred at 25 °C for 1 h. LCMS showed tert-butyl (10-(4-((4-([1,1′-biphenyl]-3-yl)-5-chloropyrimidin-2-yl)amino)piperidin-1-yl)-10-oxodecyl)carbamate (065-4) was consumed completely and the desired mass was detected. The reaction mixture was concentrated under reduced pressure to remove solvent to give the title compound (065-5) (200 mg, crude, TFA) as a yellow oil. LCMS (ES+): m/z 534.3 [M+H]+.

Synthesis of N-(10-(4-((4-([1,1′-biphenyl]-3-yl)-5-chloropyrimidin-2-yl)amino)piperidin-1-yl)-10-oxodecyl)-2-((2-(2,6-dioxopiperidin-3-yl)-1,3-dioxoisoindolin-4-yl)oxy)acetamide (Compound 66). To a solution of 2-[2-(2,6-dioxo-3-piperidyl)-1,3-dioxo-isoindolin-4-yl]oxyacetic acid (103 mg, 309 μmol, 1.00 eq) in DMF (2 mL) were added TCFH (130 mg, 463 μmol, 1.50 eq), 1-(4-((4-([1,1′-biphenyl]-3-yl)-5-chloropyrimidin-2-yl)amino)piperidin-1-yl)-10-aminodecan-1-one (065-5) (200 mg, 309 μmol, 1.00 eq, TFA) and DIEA (199 mg, 1.54 mmol, 269 μL, 5.00 eq) at 0 °C. The mixture was stirred at 0 °C for 5 min. LCMS showed 1-(4-((4-([1,1′-biphenyl]-3-yl)-5-chloropyrimidin-2-yl)amino)piperidin-1-yl)-10-aminodecan-1-one (065-5) was consumed completely and the desired mass was detected. Water (1 mL) was added to the mixture at 25 °C. Then the reaction mixture was extracted with EtOAc (0.5 mL*3). The organic layers were dried over anhydrous sodium sulfate, filtered and the filtrate was concentrated under reduced pressure to give a residue. The residue was purified by prep-HPLC (column: Phenomenex Luna C18 200*40 mm*10 μm; mobile phase: [water (FA)-ACN]; B %: 55%-90%, 8 min) to give the title compound (Compound 66) (34.2 mg, 40.3 μmol, 13.1% yield, 99.9% purity) as a white solid. LCMS (ES+): m/z 848.2 [M+H]+.

### Example 66. Synthesis of N-(4-(4-(4-((4-([1,1′-biphenyl]-3-yl)-5-chloropyrimidin-2-yl)amino)piperidine-1-carbonyl)piperidin-1-yl)-4-oxobutyl)-2-((2-(2,6-dioxopiperidin-3-yl)-1-oxoisoindolin-5-yl)oxy)acetamide (Compound 68)

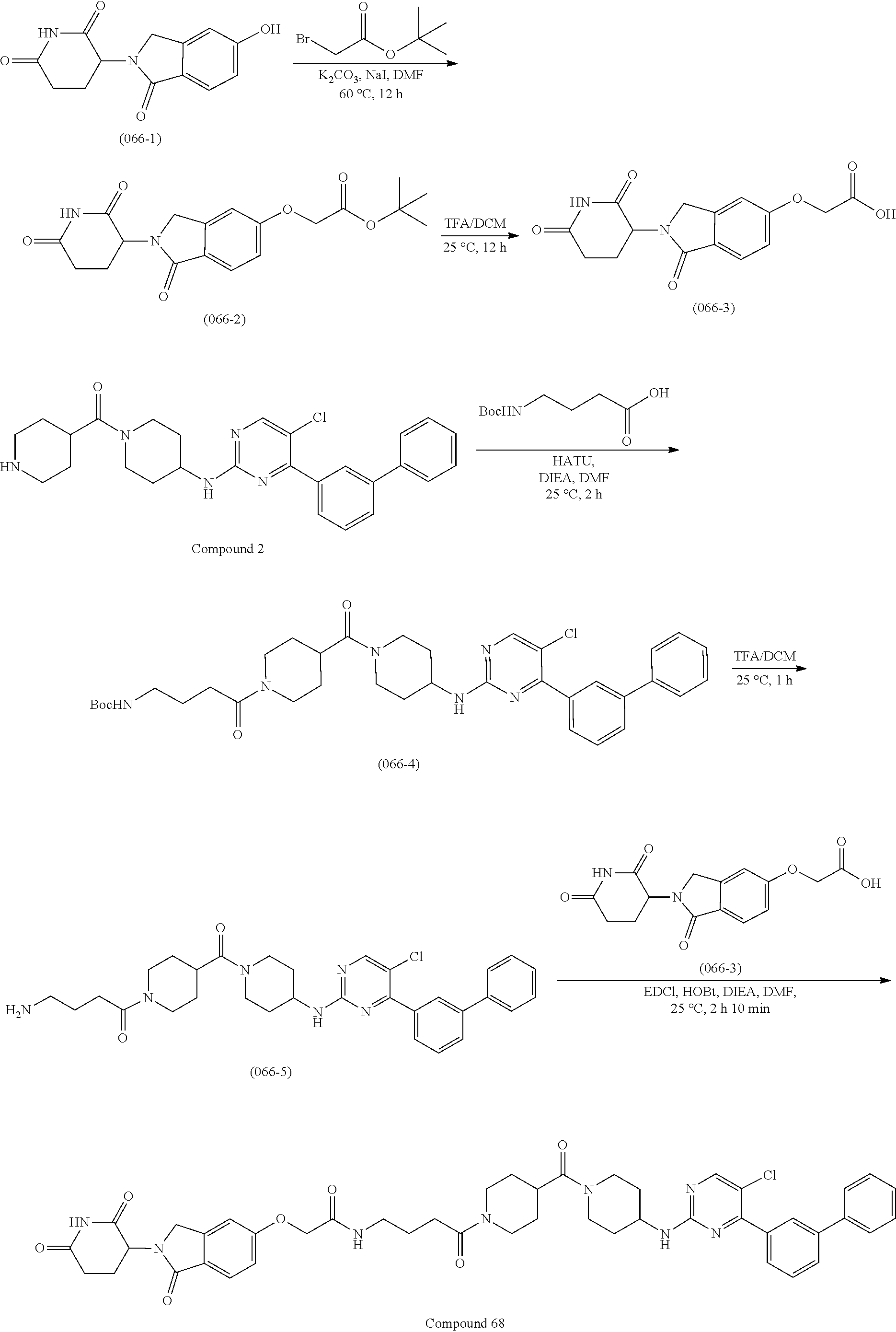

Synthesis of tert-butyl 2-((2-(2,6-dioxopiperidin-3-yl)-1-oxoisoindolin-5-yl)oxy)acetate (066-2). To a solution of 3-(5-hydroxy-1-oxoisoindolin-2-yl)piperidine-2,6-dione (066-1) (1.00 g, 3.84 mmol, 1.00 eq) and tert-butyl 2-bromoacetate (675 mg, 3.46 mmol, 511 μL, 0.900 eq) in DMF (20 mL) were added K₂CO₃ (1.06 g, 7.69 mmol, 2.00 eq) and NaI (115 mg, 769 μmol, 0.200 eq) at 25 °C. The resulting mixture was stirred at 60 °C for 12 h. LCMS showed 3-(5-hydroxy-1-oxoisoindolin-2-yl)piperidine-2,6-dione (066-1) was consumed and the desired mass was detected. H₂O (20 mL) was added to the solution, the mixture was extracted with EtOAc (20 mL*3), then the combined organic layers were washed with brine (20 mL*3), dried over Na₂SO₄, then concentrated in vacuo to give the crude product. The mixture was purified by MPLC (SiO₂, Petroleum ether:Ethyl acetate=1:0 to 1:1) to give the title compound (066-2) (850 mg, 2.27 mmol, 59.1% yield) as a white solid. LCMS (ES⁺): m/z 375.0 [M+H]⁺.

Synthesis of 2-((2-(2,6-dioxopiperidin-3-yl)-1-oxoisoindolin-5-yl)oxy)acetic acid. To a solution of tert-butyl 2-((2-(2,6-dioxopiperidin-3-yl)-1-oxoisoindolin-5-yl)oxy)acetate (066-2) (850 mg, 2.27 mmol, 1.00 eq) in DCM (8 mL) was added TFA (1.6 mL) at 25 °C. The resulting mixture was stirred at 25 °C for 12 h. LCMS showed tert-butyl 2-((2-(2,6-dioxopiperidin-3-yl)-1-oxoisoindolin-5-yl)oxy)acetate (066-2) was consumed and the desired mass was detected. The mixture was concentrated in vacuo to give the title compound (066-3) (510 mg, crude) as a yellow solid. LCMS (ES⁺): m/z 319.0 [M+H]+.

Synthesis of tert-butyl (4-(4-(4-((4-([1,1′-biphenyl]-3-yl)-5-chloropyrimidin-2-yl)amino)piperidine-1-carbonyl)piperidin-1-yl)-4-oxobutyl)carbamate (066-4). To a solution of 4-(tert-butoxycarbonylamino)butanoic acid (128 mg, 630 μmol, 1.00 eq) in DMF (3 mL) were added HATU (359 mg, 945 μmol, 1.50 eq), (4-((4-([1,1′-biphenyl]-3-yl)-5-chloropyrimidin-2-yl)amino)piperidin-1-yl)(piperidin-4-yl)methanone (Compound 2) (300 mg, 630 μmol, 1.00 eq) and DIEA (489 mg, 3.78 mmol, 659 μL, 6.00 eq) at 25 °C. The mixture was stirred at 25 °C for 2 h. LCMS showed (4-((4-([1,1′-biphenyl]-3-yl)-5-chloropyrimidin-2-yl)amino)piperidin-1-yl)(piperidin-4-yl)methanone (Compound 2) was consumed completely and the desired mass was detected. Water (2 mL) was added to the mixture at 25 °C. Then the reaction mixture was extracted with EtOAc (1 mL*3). The organic layers were dried over anhydrous sodium sulfate, filtered and the filtrate was concentrated under reduced pressure to give a residue. The residue was purified by prep-TLC (SiO₂, Ethyl acetate:Petroleum ether=1:1) to give the title compound (066-4) (340 mg, 514 μmol, 81.6% yield) as a yellow oil. LCMS (ES+): m/z 661.3 [M+H]+.

Synthesis of 1-(4-(4-((4-([1,1′-biphenyl]-3-yl)-5-chloropyrimidin-2-yl)amino)piperidine-1-carbonyl)piperidin-1-yl)-4-aminobutan-1-one (066-5). To a solution of tert-butyl (4-(4-(4-((4-([1,1′-biphenyl]-3-yl)-5-chloropyrimidin-2-yl)amino)piperidine-1-carbonyl)piperidin-1-yl)-4-oxobutyl)carbamate (066-4) (200 mg, 302 μmol, 1.00 eq) in DCM (6 mL) was added TFA (3.08 g, 27.0 mmol, 2.00 mL, 89.3 eq) at 25 °C. The mixture was stirred at 25 °C for 1 h. LCMS showed tert-butyl (4-(4-(4-((4-([1,1′-biphenyl]-3-yl)-5-chloropyrimidin-2-yl)amino)piperidine-1-carbonyl)piperidin-1-yl)-4-oxobutyl)carbamate (066-4) was consumed completely and the desired mass was detected. The reaction mixture was concentrated under reduced pressure to remove solvent to give the title compound (066-5) (204 mg, crude, TFA) as a yellow oil. LCMS (ES+): m/z 561.4 [M+H]+.

Synthesis of N-(4-(4-(4-((4-([1,1′-biphenyl]-3-yl)-5-chloropyrimidin-2-yl)amino)piperidine-1-carbonyl)piperidin-1-yl)-4-oxobutyl)-2-((2-(2,6-dioxopiperidin-3-yl)-1-oxoisoindolin-5-yl)oxy)acetamide (Compound 68). To a solution of 2-[2-(2,6-dioxo-3-piperidyl)-1-oxo-isoindolin-5-yl]oxyacetic acid (55.0 mg, 173 μmol, 1.00 eq) (066-3) in DMF (4 mL) were added EDCI (49.7 mg, 259 μmol, 1.50 eq) and DIEA (67.0 mg, 518 μmol, 90.3 μL, 3.00 eq) at 25 °C. The mixture was stirred at 25 °C for 5 min. HOBt (35.0 mg, 259 μmol, 1.50 eq) added to the mixture at 25 °C. The mixture was stirred at 25 °C for 5 min. Then 1-(4-(4-((4-([1,1′-biphenyl]-3-yl)-5-chloropyrimidin-2-yl)amino)piperidine-1-carbonyl)piperidin-1-yl)-4-aminobutan-1-one (066-5) (140 mg, 207 μmol, 1.20 eq, TFA) in DMF (1 mL) added to the mixture at 25 °C. The mixture was stirred at 25 °C for 2 h. LCMS showed the desired mass was detected. Water (4 mL) was added to the mixture at 25 °C. Then the mixture was extracted with EtOAc (3 mL*3). The organic layers were dried over anhydrous sodium sulfate, filtered and the filtrate was concentrated under reduced pressure to give a residue. The residue was purified by prep-HPLC (column: Phenomenex Luna C18 75*30 mm*3 μm; mobile phase: [water (FA)-ACN]; B %: 25%-65%, 8 min) to give the title compound (Compound 68) (10.0 mg, 11.6 μmol, 6.72% yield, 100% purity) as a white solid. LCMS (ES+): m/z 861.2 [M+H]+.

### Example 67. Synthesis of N-(5-(4-(4-((4-([1,1′-biphenyl]-3-yl)-5-chloropyrimidin-2-yl)amino)piperidine-1-carbonyl)piperidin-1-yl)-5-oxopentyl)-2-((2-(2,6-dioxopiperidin-3-yl)-1-oxoisoindolin-4-yl)oxy)acetamide (Compound 69)

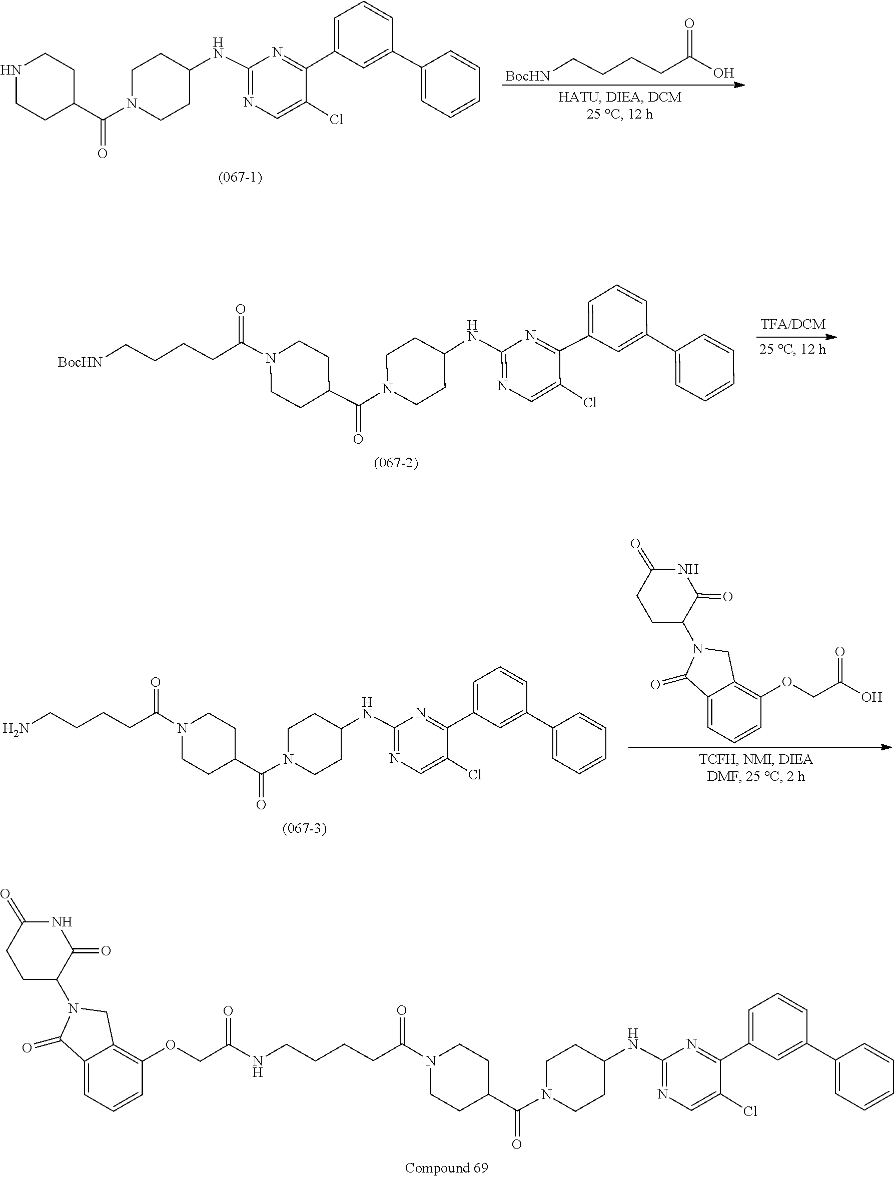 [SMILES: O=C(C1CCNCC1)N1CCC(Nc2ncc(Cl)c(-c3cccc(-c4ccccc4)c3)n2)CC1 ; CC(C)(C)OC(=O)NCCCCC(=O)O ; CC(C)(C)OC(=O)NCCCCC(=O)N1CCC(C(=O)N2CCC(Nc3ncc(Cl)c(-c4cccc(-c5ccccc5)c4)n3)CC2)CC1 ; NCCCCC(=O)N1CCC(C(=O)N2CCC(Nc3ncc(Cl)c(-c4cccc(-c5ccccc5)c4)n3)CC2)CC1 ; O=C(O)COc1cccc2c1CN(C1CCC(=O)NC1=O)C2=O ; O=C(COc1cccc2c1CN(C1CCC(=O)NC1=O)C2=O)NCCCCC(=O)N1CCC(C(=O)N2CCC(Nc3ncc(Cl)c(-c4cccc(-c5ccccc5)c4)n3)CC2)CC1]

Synthesis of tert-butyl (5-(4-(4-((4-([1,1′-biphenyl]-3-yl)-5-chloropyrimidin-2-yl)amino)piperidine-1-carbonyl)piperidin-1-yl)-5-oxopentyl)carbamate (067-2). To a solution of (4-((4-([1,1′-biphenyl]-3-yl)-5-chloropyrimidin-2-yl)amino)piperidin-1-yl)(piperidin-4-yl)methanone (Compound 2) (067-1) (600 mg, 1.02 mmol, 1.00 eq, TFA) in DCM (12 mL) were added 5-((tert-butoxycarbonyl)amino)pentanoic acid (265 mg, 1.22 mmol, 1.20 eq), HATU (580 mg, 1.53 mmol, 1.50 eq) and DIEA (657 mg, 5.08 mmol, 886 μL, 5.00 eq) at 25 °C. The mixture was stirred at 25 °C for 12 h. LCMS showed (4-((4-([1,1′-biphenyl]-3-yl)-5-chloropyrimidin-2-yl)amino)piperidin-1-yl)(piperidin-4-yl)methanone (Compound 2) (067-1) was consumed completely and the desired mass was detected. Water (10 mL) was added to reaction, the aqueous phase was extracted with EtOAc (5 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 0:1) to give the title compound (067-2) (600 mg, 889 μmol, 87.4% yield) as a white solid. LCMS (ES+): m/z 675.3 [M+H]+.

Synthesis of 1-(4-(4-((4-([1,1′-biphenyl]-3-yl)-5-chloropyrimidin-2-yl)amino)piperidine-1-carbonyl)piperidin-1-yl)-5-aminopentan-1-one (067-3). A solution of tert-butyl (5-(4-(4-((4-([1,1′-biphenyl]-3-yl)-5-chloropyrimidin-2-yl)amino)piperidine-1-carbonyl)piperidin-1-yl)-5-oxopentyl)carbamate (067-2) (600 mg, 889 μmol, 1.00 eq) in TFA (2 mL) and DCM (10 mL) was stirred at 25 °C for 12 h. LCMS showed tert-butyl (5-(4-(4-((4-([1,1′-biphenyl]-3-yl)-5-chloropyrimidin-2-yl)amino)piperidine-1-carbonyl)piperidin-1-yl)-5-oxopentyl)carbamate (067-2) was consumed completely and the desired mass was detected. The reaction mixture was concentrated under reduced pressure to give the title compound (067-3) (600 mg, crude, TFA) as a yellow oil. LCMS (ES+): m/z 575.3 [M+H]+.

Synthesis of N-(5-(4-(4-((4-([1,1′-biphenyl]-3-yl)-5-chloropyrimidin-2-yl)amino)piperidine-1-carbonyl)piperidin-1-yl)-5-oxopentyl)-2-((2-(2,6-dioxopiperidin-3-yl)-1-oxoisoindolin-4-yl)oxy)acetamide (Compound 69). To a solution of 1-(4-(4-((4-([1,1′-biphenyl]-3-yl)-5-chloropyrimidin-2-yl)amino)piperidine-1-carbonyl)piperidin-1-yl)-5-aminopentan-1-one (067-3) (230 mg, 334 μmol, 1.00 eq, TFA) and 2-((2-(2,6-dioxopiperidin-3-yl)-1-oxoisoindolin-4-yl)oxy)acetic acid (127 mg, 400 μmol, 1.20 eq) (prepared by methods analogous to those shown in Example 65 or 66) in DMF (8 mL) were added DIEA (216 mg, 1.67 mmol, 291 μL, 5.00 eq), TCFH (281 mg, 1.00 mmol, 3.00 eq) and NMI (30.1 mg, 367 μmol, 29.3 μL, 1.10 eq) at 25 °C. The mixture was stirred at 25 °C for 2 h. LCMS showed 1-(4-(4-((4-([1,1′-biphenyl]-3-yl)-5-chloropyrimidin-2-yl)amino)piperidine-1-carbonyl)piperidin-1-yl)-5-aminopentan-1-one (067-3) was consumed completely and the desired mass was detected. Water (5 mL) was added to reaction, the aqueous phase was extracted with EtOAc (4 mL*3), the combined organic layers were dried over Na₂SO₄, filtered and the filtrate was concentrated under reduced pressure. The residue was purified by prep-HPLC (column: Phenomenex luna C18 80*40 mm*3 μm; mobile phase: [water (FA)-ACN]; B %: 35%-75%, 8 min) to give the title compound (Compound 69) (110 mg, 119 mol, 35.7% yield, 95.0% purity) as a white solid. LCMS (ES+): m/z 875.2 [M+H]+.

### Example 68. Synthesis of 3-(5-(1′-(2-((S)-3-((4-([1,1′-biphenyl]-3-yl)-5-chloropyrimidin-2-yl)amino)piperidin-1-yl)-2-oxoethyl)-[1,4′-bipiperidin]-4-yl)-1-oxoisoindolin-2-yl)piperidine-2,6-dione (Compound 70)

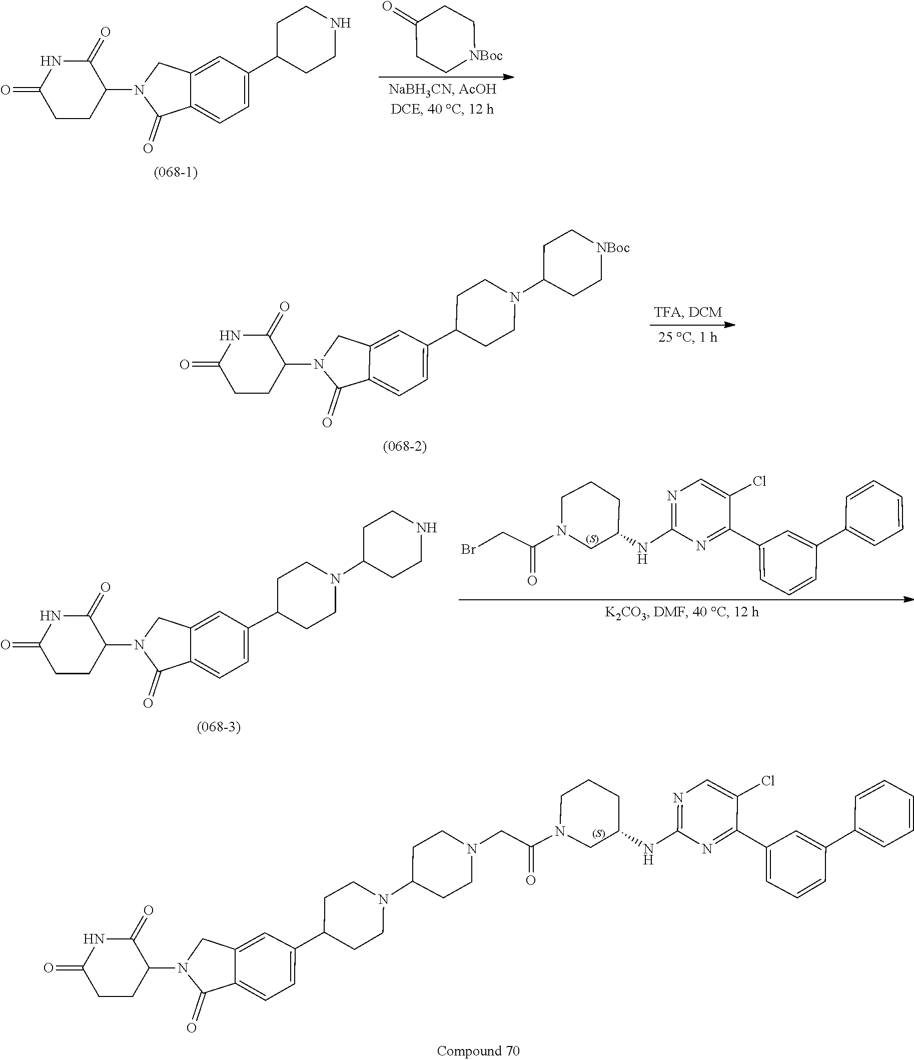

Synthesis of tert-butyl 4-(2-(2,6-dioxopiperidin-3-yl)-1-oxoisoindolin-5-yl)-[1,4′-bipiperidine]-1′-carboxylate (068-2). To a solution of 3-(1-oxo-5-(piperidin-4-yl)isoindolin-2-yl)piperidine-2,6-dione (068-1) (1.80 g, 5.50 mmol, 1.00 eq) in DCE (20.0 mL) were added AcOH (1.65 g, 27.4 mmol, 1.57 mL, 5.00 eq) and tert-butyl 4-oxopiperidine-1-carboxylate (7.67 g, 38.4 mmol, 7.00 eq) at 25 °C, then the mixture was stirred at 25 °C for 0.5 h, then NaBH₃CN (1.73 g, 27.4 mmol, 5.00 eq) was added to the mixture at 25 °C. The mixture was stirred at 40 °C for 11.5 h. LCMS showed 3-(1-oxo-5-(piperidin-4-yl)isoindolin-2-yl)piperidine-2,6-dione (068-1) remained and the desired mass was detected. The reaction mixture was diluted with H₂O (40.0 mL) and extracted with EtOAc (50.0 mL*3). The combined organic layers were washed with brine 50.0 mL, dried over Na₂SO₄, filtered and concentrated under reduced pressure. The residue was purified by column chromatography (SiO₂, DCM/MeOH=10/1 to 5/1) to give the title compound (068-2) (1.20 g, 2.16 mmol, 39.2% yield, 91.8% purity) as a white solid. LCMS (ES+): m/z 511.4 [M+H]+.

Synthesis of 3-(5-([1,4′-bipiperidin]-4-yl)-1-oxoisoindolin-2-yl)piperidine-2,6-dione (068-3). To a solution of tert-butyl 4-(2-(2,6-dioxopiperidin-3-yl)-1-oxoisoindolin-5-yl)-[1,4′-bipiperidine]-1′-carboxylate (068-2) (1.20 g, 2.35 mmol, 1.00 eq) in DCM (9.00 mL) was added TFA (4.62 g, 40.5 mmol, 3.00 mL, 17.2 eq) at 25 °C. The mixture was stirred at 25 °C for 1 h. LCMS showed 4-(2-(2,6-dioxopiperidin-3-yl)-1-oxo-2,3-dihydro-1H-inden-5-yl)-[1,4′-bipiperidine]-1′-carboxylate (068-2) was consumed completely and the desired mass was detected. The reaction concentrated under reduced pressure to give the title compound (068-3) (2.00 g, crude) as a yellow oil. LCMS (ES+): m/z 411.3 [M+H]+.

Synthesis of 3-(5-(1′-(2-((S)-3-((4-([1,1′-biphenyl]-3-yl)-5-chloropyrimidin-2-yl)amino)piperidin-1-yl)-2-oxoethyl)-[1,4′-bipiperidin]-4-yl)-1-oxoisoindolin-2-yl)piperidine-2,6-dione (Compound 70). To a solution of 3-(5-([1,4′-bipiperidin]-4-yl)-1-oxoisoindolin-2-yl)piperidine-2,6-dione (068-3) (101 mg, 247 μmol, 2.00 eq) in DMF (5 mL) were added K₂CO₃ (51.2 mg, 370 μmol, 3.00 eq) and (S)-1-(3-((4-([1,1′-biphenyl]-3-yl)-5-chloropyrimidin-2-yl)amino)piperidin-1-yl)-2-bromoethan-1-one (60.0 mg, 123.51 μmol, 1 eq) (prepared as described in Example 71) at 25 °C. The mixture was stirred at 40 °C for 12 h. LCMS showed 3-(5-([1,4′-bipiperidin]-4-yl)-1-oxoisoindolin-2-yl)piperidine-2,6-dione (068-3) was consumed completely and the desired mass was detected. The reaction filtered and concentrated under reduced pressure. The residue was purified by prep-HPLC (column: Waters Xbridge Prep OBD C18 150*40 mm*10 μm; mobile phase: [water(NH₄HCO₃)-ACN]; B %: 40%-60%, 8 min) to give the title compound (Compound 70) (32.6 mg, 39.9 μmol, 32.3% yield, 100% purity) as a white solid. LCMS (ES+): m/z 815.3 [M+H]+.

### Example 69. Synthesis of 3-(5-(1′-(2-(4-((4-([1,1′-biphenyl]-3-yl)-5-chloropyrimidin-2-yl)amino)piperidin-1-yl)-2-oxoethyl)-[1,4′-bipiperidin]-4-yl)-1-oxoisoindolin-2-yl)piperidine-2,6-dione (Compound 71)

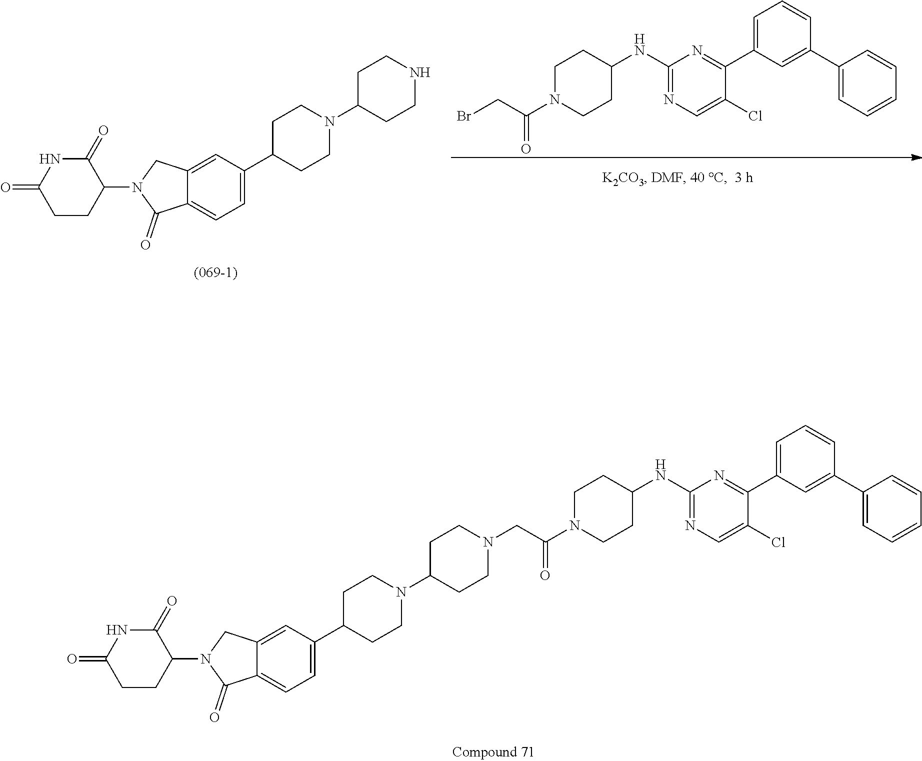 [SMILES: O=C1CCC(N2Cc3cc(C4CCN(C5CCNCC5)CC4)ccc3C2=O)C(=O)N1 ; O=C(CBr)N1CCC(Nc2ncc(Cl)c(-c3cccc(-c4ccccc4)c3)n2)CC1 ; O=C1CCC(N2Cc3cc(C4CCN(C5CCN(CC(=O)N6CCC(Nc7ncc(Cl)c(-c8cccc(-c9ccccc9)c8)n7)CC6)CC5)CC4)ccc3C2=O)C(=O)N1]

Synthesis of 3-(5-(1′-(2-(4-((4-([1,1′-biphenyl]-3-yl)-5-chloropyrimidin-2-yl)amino)piperidin-1-yl)-2-oxoethyl)-[1,4′-bipiperidin]-4-yl)-1-oxoisoindolin-2-yl)piperidine-2,6-dione (Compound 71). To a solution of 3-(5-([1,4′-bipiperidin]-4-yl)-1-oxoisoindolin-2-yl)piperidine-2,6-dione (069-1) (100 mg, 205 μmol, 1.00 eq) (as prepared in Example 68) in DMF (2 mL) were added K₂CO₃ (85.3 mg, 617 μmol, 3.00 eq) and 1-(4-((4-([1,1′-biphenyl]-3-yl)-5-chloropyrimidin-2-yl)amino)piperidin-1-yl)-2-bromoethan-1-one (84.5 mg, 206 μmol, 1.00 eq) at 25 °C. The mixture was stirred at 40 °C for 3 h. LCMS showed that 3-(5-([1,4′-bipiperidin]-4-yl)-1-oxoisoindolin-2-yl)piperidine-2,6-dione (069-1) was consumed completely and the desired mass was detected. The reaction mixture was filtered and the filtrate was concentrated under reduced pressure. The residue was purified by prep-HPLC (column: Phenomenex luna C18 80*40 mm*3 μm; mobile phase: water(FA)-ACN; B %: 15%-55%, 8 min) to give the title compound (Compound 71) (23.7 mg, 27.8 μmol, 13.5% yield, 99.0% purity, 0.58 FA) as a white solid. LCMS (ES+): m/z 815.3 [M+H]+.

### Example 70. Synthesis of 3-(5-(1′-(2-(4-((4-([1,1′-biphenyl]-3-yl)-5-chloropyrimidin-2-yl)amino)piperidin-1-yl)-2-oxoethyl)-[1,4′-bipiperidin]-4-yl)-3-methyl-2-oxo-2,3-dihydro-1H-benzo[d]imidazol-1-yl)piperidine-2,6-dione (Compound 72)

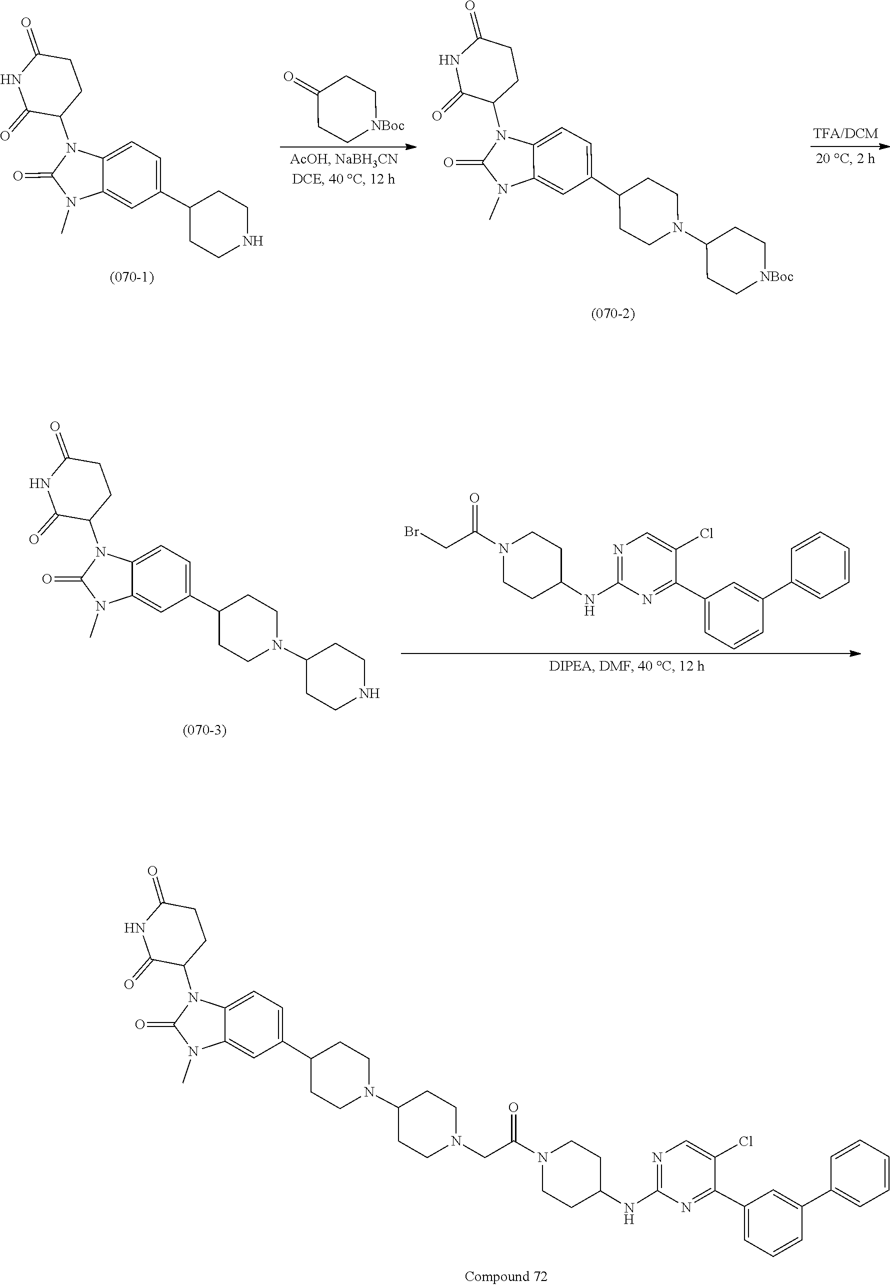 [SMILES: Cn1c(=O)n(C2CCC(=O)NC2=O)c2ccc(C3CCNCC3)cc21 ; CC(C)(C)OC(=O)N1CCC(=O)CC1 ; Cn1c(=O)n(C2CCC(=O)NC2=O)c2ccc(C3CCN(C4CCN(C(=O)OC(C)(C)C)CC4)CC3)cc21 ; Cn1c(=O)n(C2CCC(=O)NC2=O)c2ccc(C3CCN(C4CCNCC4)CC3)cc21 ; O=C(CBr)N1CCC(Nc2ncc(Cl)c(-c3cccc(-c4ccccc4)c3)n2)CC1 ; Cn1c(=O)n(C2CCC(=O)NC2=O)c2ccc(C3CCN(C4CCN(CC(=O)N5CCC(Nc6ncc(Cl)c(-c7cccc(-c8ccccc8)c7)n6)CC5)CC4)CC3)cc21]

Synthesis of tert-butyl 4-(1-(2,6-dioxopiperidin-3-yl)-3-methyl-2-oxo-2,3-dihydro-1H-benzo[d]imidazol-5-yl)-[1,4′-bipiperidine]-1′-carboxylate (070-2). To a solution of 3-(3-methyl-2-oxo-5-(piperidin-4-yl)-2,3-dihydro-1H-benzo[d]imidazol-1-yl)piperidine-2,6-dione (070-1) (6.00 g, 17.5 mmol, 1.00 eq) in DCE (10.0 mL) were added tert-butyl 4-oxopiperidine-1-carboxylate (24.4 g, 122 mmol, 7.00 eq) and AcOH (3.16 g, 52.5 mmol, 3.01 mL, 3.00 eq) at 25 °C. The mixture was stirred at 25 °C for 0.5 h, then NaBH₃CN (5.51 g, 87.6 mmol, 5.00 eq) was added to the mixture at 25 °C, the mixture was stirred at 40 °C for 11.5 h. LCMS showed 3-(3-methyl-2-oxo-5-(piperidin-4-yl)-2,3-dihydro-1H-benzo[d]imidazol-1-yl)piperidine-2,6-dione (070-1) was consumed completely and the desired mass was detected. Water (50.0 mL) was added to the solution, the mixture was extracted with ethyl acetate (50.0 mL*3), the combined organic layers were dried over Na₂SO₄, and concentrated under reduced pressure. The residue was purified by column chromatography (SiO₂, Dichloromethane/Methanol=10/1 to 5/1) to give the title compound (070-2) (1.80 g, 2.85 mmol, 16.3% yield, 83.3% purity) as a white solid. LCMS (ES+): m/z 526.2 [M+H]+.

Synthesis of 3-(5-([1,4′-bipiperidin]-4-yl)-3-methyl-2-oxo-2,3-dihydro-1H-benzo[d]imidazol-1-yl)piperidine-2,6-dione (070-3). To a solution of tert-butyl 4-(1-(2,6-dioxopiperidin-3-yl)-3-methyl-2-oxo-2,3-dihydro-1H-benzo[d]imidazol-5-yl)-[1,4′-bipiperidine]-1′-carboxylate (070-2) (1.80 g, 3.42 mmol, 1.00 eq) in DCM (3.00 mL) was added TFA (1.95 g, 17.1 mmol, 1.27 mL, 5.00 eq) at 20 °C. The mixture was stirred at 20 °C for 2 h. LCMS showed that tert-butyl 4-(1-(2,6-dioxopiperidin-3-yl)-3-methyl-2-oxo-2,3-dihydro-1H-benzo[d]imidazol-5-yl)-[1,4′-bipiperidine]-1′-carboxylate (070-2) was consumed completely and the desired mass was detected. The reaction concentrated under reduced pressure to give the title compound (070-3) (2.60 g, crude) as a white solid. LCMS (ES+): m/z 426.4 [M+H]+.

Synthesis of 3-(5-(1′-(2-(4-((4-([1,1′-biphenyl]-3-yl)-5-chloropyrimidin-2-yl)amino)piperidin-1-yl)-2-oxoethyl)-[1,4′-bipiperidin]-4-yl)-3-methyl-2-oxo-2,3-dihydro-1H-benzo[d]imidazol-1-yl)piperidine-2,6-dione (Compound 72). To a solution of 3-(5-([1,4′-bipiperidin]-4-yl)-3-methyl-2-oxo-2,3-dihydro-1H-benzo[d]imidazol-1-yl)piperidine-2,6-dione (070-3) (100 mg, 235 μmol, 1.00 eq) in DMF (2.00 mL) were added DIEA (30.3 mg, 235 μmol, 40.9 μL, 1.00 eq) and 2-bromo-1-[4-[[5-chloro-4-(3-phenylphenyl)pyrimidin-2-yl]amino]-1-piperidyl]ethanone (prepared as described in Example 83) (114 mg, 235 μmol, 1.00 eq) at 25 °C. The mixture was stirred at 40 °C for 12 h. LCMS showed that 3-(5-([1,4′-bipiperidin]-4-yl)-3-methyl-2-oxo-2,3-dihydro-1H-benzo[d]imidazol-1-yl)piperidine-2,6-dione (070-3) was consumed completely and the desired mass was detected. The reaction concentrated under reduced pressure. The residue was purified by prep-HPLC (column: Phenomenex Luna C18 75*30 mm*3 μm; mobile phase: [water(FA)-ACN]; B %: 25%-45%, 8 min) to give the title compound (Compound 72) (47.8 mg, 55.4 μmol, 23.6% yield, 100% purity, 0.680 FA) as a white solid. LCMS (ES+): m/z 830.4 [M+H]+.

### Example 71. Synthesis of 3-(5-(1-((1-(2-((S)-3-((4-([1,1′-biphenyl]-3-yl)-5-chloropyrimidin-2-yl)amino)piperidin-1-yl)-2-oxoethyl)piperidin-4-yl)methyl)piperidin-4-yl)-3-methyl-2-oxo-2,3-dihydro-1H-benzo[d]imidazol-1-yl)piperidine-2,6-dione (Compound 73)

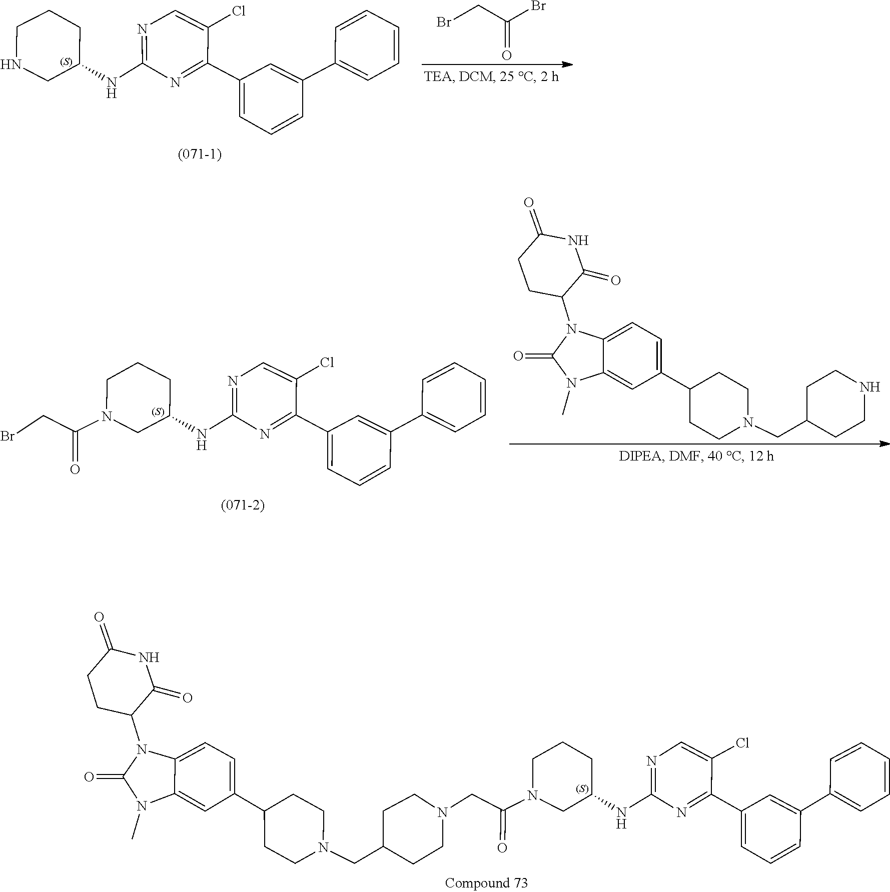

Synthesis of (S)-1-(3-((4-([1,1′-biphenyl]-3-yl)-5-chloropyrimidin-2-yl)amino)piperidin-1-yl)-2-bromoethan-1-one (071-2). To a solution of (S)-4-([1,1′-biphenyl]-3-yl)-5-chloro-N-(piperidin-3-yl)pyrimidin-2-amine (071-1) (1.50 g, 4.11 mmol, 1.00 eq) (prepared as described in Example 18) in DCM (10.0 mL) was added TEA (1.25 g, 12.3 mmol, 1.72 mL, 3.00 eq) and 2-bromoacetyl bromide (1.08 g, 5.34 mmol, 464 μL, 1.30 eq) at 25 °C. The mixture was stirred at 25 °C for 2 h. LCMS showed that (S)-4-([1,1′-biphenyl]-3-yl)-5-chloro-N-(piperidin-3-yl)pyrimidin-2-amine (071-1) was consumed completely and the desired mass was detected. Water (50.0 mL) was added to the solution, the mixture was extracted with ethyl acetate (50.0 mL*3), the combined organic layers were dried over Na₂SO₄, and the filtrate was concentrated under reduced pressure. The residue was purified by column chromatography (SiO₂, Dichloromethane/Methanol=10/1 to 5/1) to give the title compound (071-2) (1.00 g, 1.07 mmol, 25.9% yield, 51.8% purity) as a white solid. LCMS (ES+): m/z 485.0 [M+H]+.

Synthesis of 3-(5-(1-((1-(2-((S)-3-((4-([1,1′-biphenyl]-3-yl)-5-chloropyrimidin-2-yl)amino)piperidin-1-yl)-2-oxoethyl)piperidin-4-yl)methyl)piperidin-4-yl)-3-methyl-2-oxo-2,3-dihydro-1H-benzo[d]imidazol-1-yl)piperidine-2,6-dione (Compound 73). To a solution of (S)-1-(3-((4-([1,1′-biphenyl]-3-yl)-5-chloropyrimidin-2-yl)amino)piperidin-1-yl)-2-bromoethan-1-one (071-2) (200 mg, 411 μmol, 1.00 eq) in DMF (2 mL) were added DIEA (53.2 mg, 411 μmol, 71.7 μL, 1.00 eq) and 3-[3-methyl-2-oxo-5-[1-(4-piperidylmethyl)-4-piperidyl]benzimidazol-1-yl]piperidine-2,6-dione (180 mg, 411 μmol, 1.00 eq) (prepared as described in Example 47) at 25 °C. The mixture was stirred at 40 °C for 12 h. LCMS showed that (S)-1-(3-((4-([1,1′-biphenyl]-3-yl)-5-chloropyrimidin-2-yl)amino)piperidin-1-yl)-2-bromoethan-1-one (071-2) was consumed completely and the desired mass was detected. The reaction was concentrated under reduced pressure. The residue was purified by prep-HPLC (column: Phenomenex Luna C18 200*40 mm*10 μm; mobile phase: [water(FA)-ACN]; B %: 10%-45%, 8 min) to give the title compound (Compound 73) (25.5 mg, 29.1 μmol, 7.07% yield, 99.5% purity, 0.600 FA) as white solid. LCMS (ES+): m/z 844.4 [M+H]+.

### Example 72. Synthesis of 3-((4-(1-(2-(4-(4-((4-([1,1′-biphenyl]-3-yl)-5-chloropyrimidin-2-yl)amino)piperidine-1-carbonyl)piperidin-1-yl)ethyl)piperidin-4-yl)phenyl)amino)piperidine-2,6-dione (Compound 74)

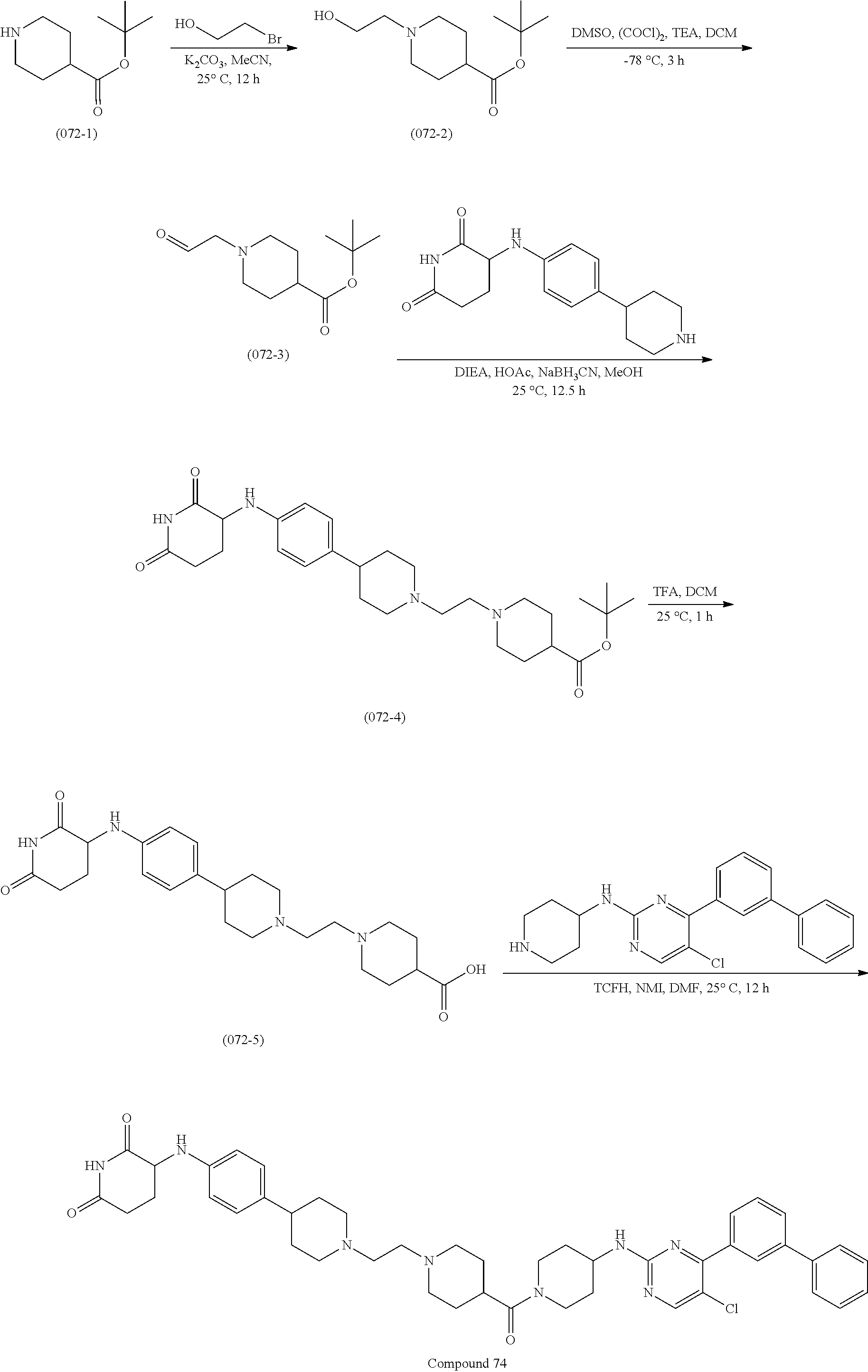

Synthesis of tert-butyl 1-(2-hydroxyethyl)piperidine-4-carboxylate (072-2). To a solution of tert-butyl piperidine-4-carboxylate (072-1) (2.00 g, 10.8 mmol, 1.00 eq) and 2-bromoethanol (1.62 g, 12.9 mmol, 919 μL, 1.20 eq) in ACN (20 mL) was added K₂CO₃ (4.48 g, 32.3 mmol, 3.00 eq) at 25 °C. The mixture was stirred at 25 °C for 12 h. LCMS showed that the desired mass was detected. The reaction mixture was filtered and the filtrate was concentrated under reduced pressure. The residue was purified by column chromatography (SiO₂, Dichloromethane/Methanol=1/0 to 0/1) to give the title compound (072-2) (1.20 g, 4.92 mmol, 49.0% yield, 94.0% purity) as a yellow oil. LCMS (ES+): m/z 230.1 [M+H]+.

Synthesis of tert-butyl 1-(2-oxoethyl)piperidine-4-carboxylate (072-3). To a solution of oxalyl dichloride (581 mg, 4.58 mmol, 400 μL, 2.10 eq) in DCM (3 mL) was added DMSO (579 mg, 7.41 mmol, 579 μL, 3.40 eq) at −78 °C under N₂ atmosphere. The mixture was stirred at −78 °C for 1 h under N₂ atmosphere. Then tert-butyl 1-(2-hydroxyethyl)piperidine-4-carboxylate (072-2) (500 mg, 2.18 mmol, 1.00 eq) was added at −78 °C. The mixture was stirred at −78 °C for 1 h. Then added TEA (1.46 g, 14.3 mmol, 2.00 mL, 6.60 eq) at −78 °C. The mixture was stirred at −78 °C for 1 h. LCMS showed the desired mass was detected. The reaction mixture was diluted with H₂O 15 mL and extracted with DCM (10 mL*3). The combined organic layers were washed with brine 10 mL, dried over Na₂SO₄, filtered and concentrated under reduced pressure to give the title compound (072-3) (460 mg, crude) as a yellow oil. LCMS (ES+): m/z 228.1 [M+H]+.

Synthesis of tert-butyl 1-(2-(4-(4-((2,6-dioxopiperidin-3-yl)amino)phenyl)piperidin-1-yl)ethyl)piperidine-4-carboxylate (072-4). To a solution of 3-[4-(4-piperidyl) anilino]piperidine-2,6-dione (232 mg, 809 μmol, 0.400 eq) in MeOH (4 mL) was added DIEA (261 mg, 2.02 mmol, 352 μL, 1.00 eq) at 25 °C to adjust pH to 8. The mixture was stirred at 25 °C for 0.5 h. Then AcOH (121 mg, 2.02 mmol, 115 μL, 1.00 eq), tert-butyl 1-(2-oxoethyl)piperidine-4-carboxylate (072-3) (460 mg, 2.02 mmol, 1.00 eq) and sodium cyanoborohydride (381 mg, 6.07 mmol, 3.00 eq) were added to the mixture at 25 °C. The mixture was stirred at 25 °C for 1.5 h. LCMS showed the desired mass was detected. The reaction mixture was diluted with H₂O 10 mL and extracted with EtOAc mL (10 mL*3). The combined organic layers were washed with aqueous NaCl 10 mL, dried over Na₂SO₄, filtered and concentrated under reduced pressure. The residue was purified by column chromatography (SiO₂, Dichloromethane/Methanol=1/0 to 10/1) to give the title compound (072-4) (360 mg, 577. μmol, 28.5% yield, 80.0% purity) as a colorless oil. LCMS (ES+): m/z 499.3 [M+H]+.

Synthesis of 1-(2-(4-(4-((2,6-dioxopiperidin-3-yl)amino)phenyl)piperidin-1-yl)ethyl)piperidine-4-carboxylic acid (072-5). To a solution of tert-butyl 1-(2-(4-(4-((2,6-dioxopiperidin-3-yl)amino)phenyl)piperidin-1-yl)ethyl)piperidine-4-carboxylate (072-4) (360 mg, 721 μmol, 1.00 eq) in DCM (5 mL) was added TFA (4.62 g, 40.5 mmol, 3 mL, 56.1 eq) at 25 °C. The mixture was stirred at 25 °C for 1 h. LCMS showed the desired mass was detected. The reaction concentrated under reduced pressure to give the title compound (072-5) (300 mg, crude) as a yellow oil. LCMS (ES+): m/z 443.2 [M+H]+.

Synthesis of 3-((4-(1-(2-(4-(4-((4-([1,1′-biphenyl]-3-yl)-5-chloropyrimidin-2-yl)amino)piperidine-1-carbonyl)piperidin-1-yl)ethyl)piperidin-4-yl)phenyl)amino)piperidine-2,6-dione (Compound 74). To a solution of 1-(2-(4-(4-((2,6-dioxopiperidin-3-yl)amino)phenyl)piperidin-1-yl)ethyl)piperidine-4-carboxylic acid (072-5) (100 mg, 225 μmol, 1.00 eq) and 5-chloro-4-(3-phenylphenyl)-N-(4-piperidyl)pyrimidin-2-amine (Compound B) (82.4 mg, 225 μmol, 1.00 eq) in DMF (3 mL) were added TCFH (69.7 mg, 248 μmol, 1.10 eq) and NMI (57.5 mg, 700 μmol, 55.8 μL, 3.10 eq) at 25 °C. The mixture was stirred at 25 °C for 12 h. LCMS showed that the desired mass was detected. The reaction mixture was diluted with H₂O 10 mL and extracted with EtOAc (10 mL*3). The combined organic layers were washed with brine 10 mL, dried over Na₂SO₄, filtered and the filtrate was concentrated under reduced pressure. The residue was purified by prep-HPLC (column: Phenomenex luna C18 80*40 mm*3 μm; mobile phase: [water (FA)-ACN]; B %: 20%-60%, 8 min) to give the title compound (Compound 74) (11.4 mg, 13.7 μmol, 6.06% yield, 97.0% purity, 0.4 FA) as a yellow solid. LCMS (ES+): m/z 789.4 [M+H]+.

### Example 73. Synthesis of 3-(5-(1′-(2-((S)-3-((4-([1,1′-biphenyl]-3-yl)-5-chloropyrimidin-2-yl)amino)piperidin-1-yl)-2-oxoethyl)-[1,4′-bipiperidin]-4-yl)-3-methyl-2-oxo-2,3-dihydro-1H-benzo[d]imidazol-1-yl)piperidine-2,6-dione (Compound 75)

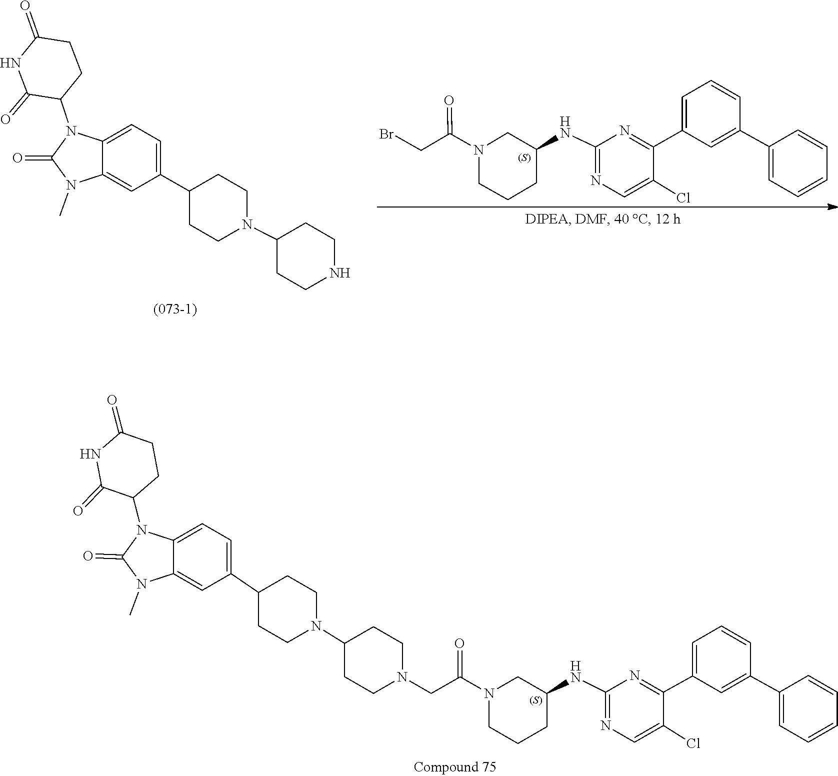

Synthesis of 3-(5-(1′-(2-((S)-3-((4-([1,1′-biphenyl]-3-yl)-5-chloropyrimidin-2-yl)amino)piperidin-1-yl)-2-oxoethyl)-[1,4′-bipiperidin]-4-yl)-3-methyl-2-oxo-2,3-dihydro-1H-benzo[d]imidazol-1-yl)piperidine-2,6-dione (Compound 75). To a solution of 3-(5-([1,4′-bipiperidin]-4-yl)-3-methyl-2-oxo-2,3-dihydro-1H-benzo[d]imidazol-1-yl)piperidine-2,6-dione (073-1) (100 mg, 235 μmol, 1.00 eq) (as prepared in Example 70) in DMF (2.00 mL) were added DIEA (91.1 mg, 705 μmol, 122 μL, 3.00 eq) and 2-bromo-1-[(3S)-3-[[5-chloro-4-(3-phenylphenyl)pyrimidin-2-yl]amino]-1-piperidyl]ethanone (114 mg, 235 μmol, 1.00 eq) (prepared as described in Example 71) at 25 °C. The mixture was stirred at 40 °C for 12 h. LCMS showed that 3-(5-([1,4′-bipiperidin]-4-yl)-3-methyl-2-oxo-2,3-dihydro-1H-benzo[d]imidazol-1-yl)piperidine-2,6-dione (073-1) was consumed completely and the desired mass was detected. The reaction concentrated under reduced pressure. The residue was purified by prep-HPLC (column: C18-6 100*30 mm*5 μm; mobile phase: [water (FA)-ACN]; B %: 10%-40%, 8 min) to give the title compound (Compound 75) (8.00 mg, 9.50 μmol, 4.04% yield, 100% purity, 0.260 FA) as a white solid. LCMS (ES+): m/z 830.4 [M+H]+.

### Example 74. cis-3-(5-((1-((1-(3-((4-([1,1′-biphenyl]-3-yl)-5-chloropyrimidin-2-yl)amino)cyclohexane-1-carbonyl)piperidin-4-yl)methyl)piperidin-4-yl)oxy)-1-oxoisoindolin-2-yl)piperidine-2,6-dione (Compound 76)

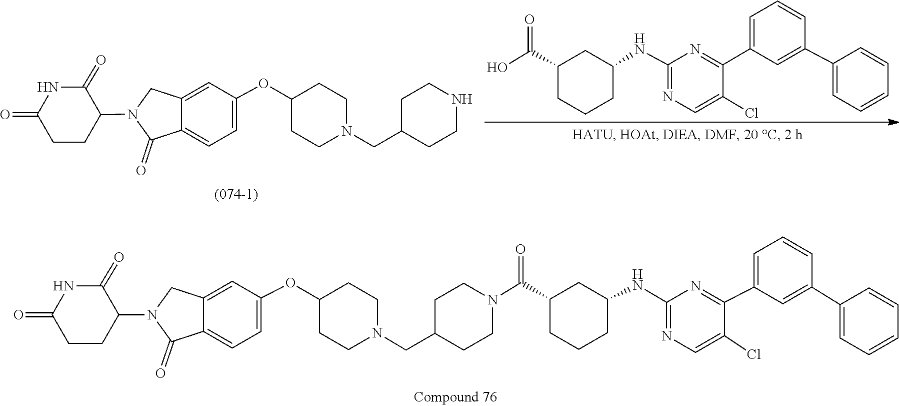 [SMILES: O=C1CCC(N2Cc3cc(OC4CCN(CC5CCNCC5)CC4)ccc3C2=O)C(=O)N1 ; O=C(O)[C@H]1CCC[C@@H](Nc2ncc(Cl)c(-c3cccc(-c4ccccc4)c3)n2)C1 ; O=C1CCC(N2Cc3cc(OC4CCN(CC5CCN(C(=O)[C@H]6CCC[C@@H](Nc7ncc(Cl)c(-c8cccc(-c9ccccc9)c8)n7)C6)CC5)CC4)ccc3C2=O)C(=O)N1]

Synthesis of cis-3-(5-((1-((1-(3-((4-([1,1′-biphenyl]-3-yl)-5-chloropyrimidin-2-yl)amino)cyclohexane-1-carbonyl)piperidin-4-yl)methyl)piperidin-4-yl)oxy)-1-oxoisoindolin-2-yl)piperidine-2,6-dione (Compound 76). To a solution of 3-(1-oxo-5-((1-(piperidin-4-ylmethyl)piperidin-4-yl)oxy)isoindolin-2-yl)piperidine-2,6-dione (074-1) (160 mg, 289 μmol, 1.00 eq, TFA) (prepared as in Example 83) in DMF (4 mL) was added DIEA (186 mg, 1.44 mmol, 251 μL, 5.00 eq) at 20 °C, the mixture was stirred at 20 °C for 0.5 h, then cis-3-((4-([1,1′-biphenyl]-3-yl)-5-chloropyrimidin-2-yl)amino)cyclohexane-1-carboxylic acid (141 mg, 346 μmol, 1.20 eq), HATU (132 mg, 346 μmol, 1.20 eq) and HOAt (19.6 mg, 144 μmol, 20.2 μL, 0.500 eq) were added to the mixture at 20 °C, the mixture was stirred at 20 °C for 2 h. LCMS showed 3-(1-oxo-5-((1-(piperidin-4-ylmethyl)piperidin-4-yl)oxy)isoindolin-2-yl)piperidine-2,6-dione (074-1) was consumed completely and the desired mass was detected. The reaction was filtered and the filtrate was sent to be purified by prep-HPLC directly. It was purified by prep-HPLC (column: Waters Xbridge Prep OBD C18 150*40 mm*10 μm; mobile phase: [water(NH₄HCO₃)-ACN]; B %: 35%-75%, 8 min) to give the title compound (Compound 76) (73.6 mg, 85.1 μmol, 24.2% yield, 97.6% purity) as a white solid. LCMS (ES+): m/z 830.6 [M+H]+.

### Example 75. Synthesis of cis-3-(5-((1-(1-(3-((4-([1,1′-biphenyl]-3-yl)-5-chloropyrimidin-2-yl)amino)cyclohexane-1-carbonyl)piperidine-4-carbonyl)piperidin-4-yl)oxy)-1-oxoisoindolin-2-yl)piperidine-2,6-dione (Compound 77)

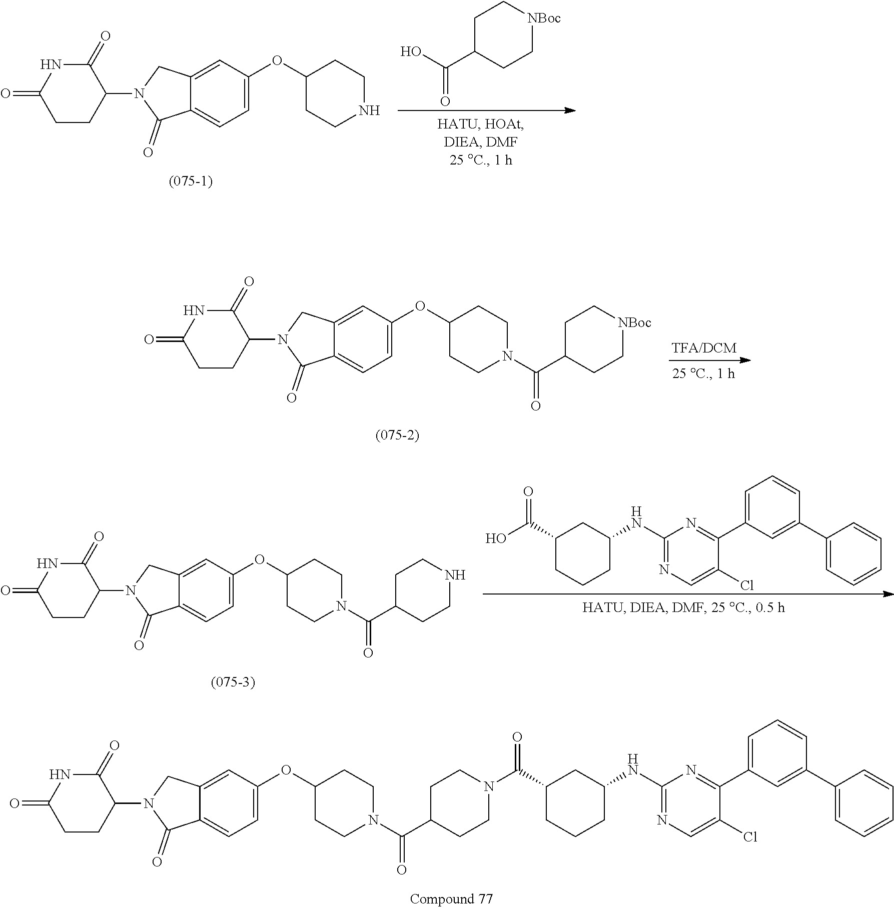 [SMILES: O=C1CCC(N2Cc3cc(OC4CCNCC4)ccc3C2=O)C(=O)N1 ; CC(C)(C)OC(=O)N1CCC(C(=O)O)CC1 ; CC(C)(C)OC(=O)N1CCC(C(=O)N2CCC(Oc3ccc4c(c3)CN(C3CCC(=O)NC3=O)C4=O)CC2)CC1 ; O=C1CCC(N2Cc3cc(OC4CCN(C(=O)C5CCNCC5)CC4)ccc3C2=O)C(=O)N1 ; O=C(O)[C@H]1CCC[C@@H](Nc2ncc(Cl)c(-c3cccc(-c4ccccc4)c3)n2)C1 ; O=C1CCC(N2Cc3cc(OC4CCN(C(=O)C5CCN(C(=O)[C@H]6CCC[C@@H](Nc7ncc(Cl)c(-c8cccc(-c9ccccc9)c8)n7)C6)CC5)CC4)ccc3C2=O)C(=O)N1]

Synthesis of tert-butyl 4-(4-((2-(2,6-dioxopiperidin-3-yl)-1-oxoisoindolin-5-yl)oxy)piperidine-1-carbonyl)piperidine-1-carboxylate (075-2). To a solution of 1-(tert-butoxycarbonyl)piperidine-4-carboxylic acid (241 mg, 1.05 mmol, 1.20 eq) in DMF (3 mL) were added 3-(1-oxo-5-(piperidin-4-yloxy)isoindolin-2-yl)piperidine-2,6-dione (075-1) (400 mg, 875 μmol, 1.00 eq, TFA) (prepared as described in Example 83), DIEA (565 mg, 4.37 mmol, 762 μL, 5.00 eq), HOAt (59.5 mg, 437 μmol, 61.2 μL, 0.500 eq) and HATU (499 mg, 1.31 mmol, 1.50 eq) at 25 °C. The mixture was stirred at 25 °C for 1 h. LCMS showed 3-(1-oxo-5-(piperidin-4-yloxy)isoindolin-2-yl)piperidine-2,6-dione (075-1) was consumed, desired target MS was detected. H₂O (5 mL) was added to the solution, the mixture was extracted with EtOAc (5 mL*3), then the combined organic phase was washed with brine (20 mL*3), the combined organic lays were dried over Na₂SO₄, then concentrated in vacuo. The mixture was purified by prep-TLC (SiO₂, PE:EtOAc=0:1) to give the title compound (075-2) (290 mg, 523 μmol, 59.8% yield) as a yellow oil. LCMS (ES+): m/z 455.2 [M+H−100]+.

Synthesis of 3-(1-oxo-5-((1-(piperidine-4-carbonyl)piperidin-4-yl)oxy)isoindolin-2-yl)piperidine-2,6-dione (075-3). A solution of tert-butyl 4-(4-((2-(2,6-dioxopiperidin-3-yl)-1-oxoisoindolin-5-yl)oxy)piperidine-1-carbonyl)piperidine-1-carboxylate (075-2) (290 mg, 523 μmol, 1.00 eq) in TFA (1 mL) and DCM (3 mL) was stirred at 25 °C for 1 h. LCMS showed tert-butyl 4-(4-((2-(2,6-dioxopiperidin-3-yl)-1-oxoisoindolin-5-yl)oxy)piperidine-1-carbonyl)piperidine-1-carboxylate (075-2) was consumed, desired target MS was detected. The mixture was concentrated in vacuo to give the title compound (075-3) (290 mg, crude, TFA) as a yellow oil. LCMS (ES+): m/z 455.2 [M+H]+.

Synthesis of cis-3-(5-((1-(1-(3-((4-([1,1′-biphenyl]-3-yl)-5-chloropyrimidin-2-yl)amino)cyclohexane-1-carbonyl)piperidine-4-carbonyl)piperidin-4-yl)oxy)-1-oxoisoindolin-2-yl)piperidine-2,6-dione (Compound 77). To a solution of cis-3-((4-([1,1′-biphenyl]-3-yl)-5-chloropyrimidin-2-yl)amino)cyclohexane-1-carboxylic acid (201 mg, 492 μmol, 1.00 eq) (prepared by methods analogous to those shown in Example 22) in DMF (2 mL) were added HATU (225 mg, 591 μmol, 1.20 eq) and DIEA (318 mg, 2.46 mmol, 428.90 μL, 5.00 eq) at 25 °C, then 3-[1-oxo-5-[[1-(piperidine-4-carbonyl)-4-piperidyl]oxy]isoindolin-2-yl]piperidine-2,6-dione (075-3) (280 mg, 492 μmol, 1.00 eq, TFA) was added to the mixture, the mixture was stirred at 25 °C for 0.5 h. LCMS showed 3-[l-oxo-5-[[1-(piperidine-4-carbonyl)-4-piperidyl]oxy]isoindolin-2-yl]piperidine-2,6-dione (075-3) was consumed and the desired mass was detected. H₂O (3 mL) was added to the solution, the mixture was extracted with EtOAc (3 mL*3), then the combined organic layers were dried over Na₂SO₄, then concentrated in vacuo. The residue was purified by prep-HPLC (column: Phenomenex Luna C18 75*30 mm*3 μm; mobile phase: [water(FA)-ACN]; B %: 55%-90%, 8 min) to give the title compound (Compound 77) (81.1 mg, 95.7 μmol, 19.4% yield, 99.7% purity) as a white solid. LCMS (ES+): m/z 844.5 [M+H]+.

### Example 76. Synthesis of 3-(5-(1-(2-(4-((S)-3-((4-([1,1′-biphenyl]-3-yl)-5-chloropyrimidin-2-yl)amino)piperidine-1-carbonyl)piperidin-1-yl)ethyl)piperidin-4-yl)-1-oxo-2,3-dihydro-1H-inden-2-yl)piperidine-2,6-dione (Compound 78)

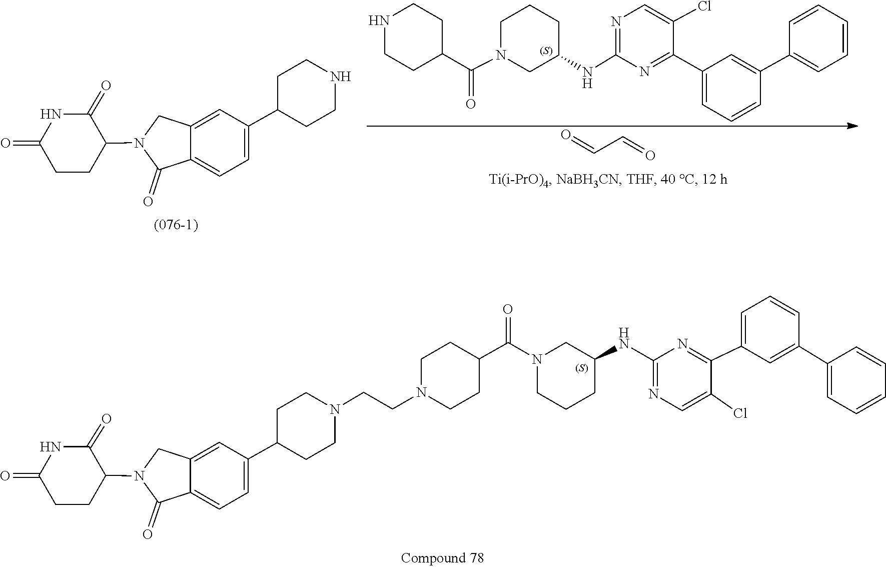

Synthesis of 3-(5-(1-(2-(4-((S)-3-((4-([1,1′-biphenyl]-3-yl)-5-chloropyrimidin-2-yl)amino)piperidine-1-carbonyl)piperidin-1-yl)ethyl)piperidin-4-yl)-1-oxo-2,3-dihydro-1H-inden-2-yl)piperidine-2,6-dione (Compound 78). To a solution of (S)-(3-((4-([1,1′-biphenyl]-3-yl)-5-chloropyrimidin-2-yl)amino)piperidin-1-yl)(piperidin-4-yl)methanone (076-1) (200 mg, 420 μmol, 1.00 eq) (as prepared in Example 44) and 3-[1-oxo-5-(4-piperidyl)isoindolin-2-yl]piperidine-2,6-dione (150 mg, 458 μmol, 1.09 eq) in THF (5.00 mL) were added oxaldehyde (24.3 mg, 420 μmol, 21.9 μL, 1.00 eq) and Ti(i-PrO)₄ (597 mg, 2.10 mmol, 620 μL, 5.00 eq) at 25 °C, the mixture was stirred at 25 °C for 0.5 h, then NaBH₃CN (132 mg, 2.10 mmol, 5.00 eq) was added to the mixture at 25 °C. The mixture was stirred at 40 °C for 11.5 h. LCMS showed that 3-(1-oxo-5-(piperidin-4-yl)isoindolin-2-yl)piperidine-2,6-dione (076-1) was consumed completely and the desired mass was detected. The reaction mixture was diluted with H₂O 20.0 mL and extracted with EtOAc (30.0 mL*3). The combined organic layers were washed with brine 20.0 mL, dried over Na₂SO₄, filtered and concentrated under reduced pressure. The residue was purified by prep-HPLC (column: Phenomenex luna C18 80*40 mm*3 μm; mobile phase: [water(FA)-ACN]; B %: 25%-65%, 8 min) to give the title compound (Compound 78) (24.6 mg, 28.1 μmol, 6.70% yield, 97.0% purity, 0.400 FA) as a white solid. LCMS (ES+): m/z 829.4 [M+H]+

### Example 77. Synthesis of cis-3-(5-((1-(2-(1-(3-((4-([1,1′-biphenyl]-3-yl)-5-chloropyrimidin-2-yl)amino)cyclohexane-1-carbonyl)piperidin-4-yl)ethyl)piperidin-4-yl)oxy)-1-oxoisoindolin-2-yl)piperidine-2,6-dione (Compound 79)

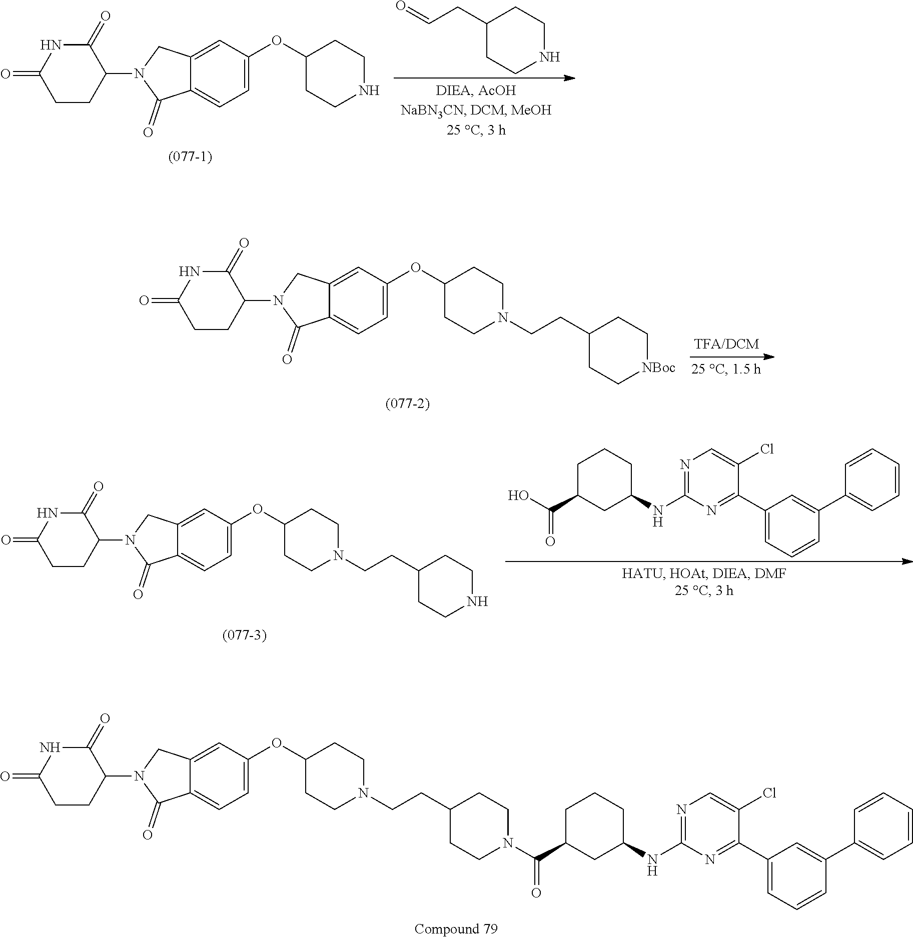 [SMILES: O=C1CCC(N2Cc3cc(OC4CCNCC4)ccc3C2=O)C(=O)N1 ; O=CCC1CCNCC1 ; CC(C)(C)OC(=O)N1CCC(CCN2CCC(Oc3ccc4c(c3)CN(C3CCC(=O)NC3=O)C4=O)CC2)CC1 ; O=C1CCC(N2Cc3cc(OC4CCN(CCC5CCNCC5)CC4)ccc3C2=O)C(=O)N1 ; O=C(O)[C@H]1CCC[C@@H](Nc2ncc(Cl)c(-c3cccc(-c4ccccc4)c3)n2)C1 ; O=C1CCC(N2Cc3cc(OC4CCN(CCC5CCN(C(=O)[C@H]6CCC[C@@H](Nc7ncc(Cl)c(-c8cccc(-c9ccccc9)c8)n7)C6)CC5)CC4)ccc3C2=O)C(=O)N1]

Synthesis of tert-butyl 4-(2-(4-((2-(2,6-dioxopiperidin-3-yl)-1-oxoisoindolin-5-yl)oxy)piperidin-1-yl)ethyl)piperidine-1-carboxylate (077-2). To a solution of 3-(1-oxo-5-(piperidin-4-yloxy)isoindolin-2-yl)piperidine-2,6-dione (077-1) (300 mg, 656 μmol, 1.00 eq, TFA) (prepared as described in Example 83) in DCM (3 mL) and MeOH (3 mL) were added tert-butyl 4-(2-oxoethyl)piperidine-1-carboxylate (447 mg, 1.97 mmol, 3.00 eq) at 25 °C. DIEA (84.8 mg, 656 μmol, 114 μL, 1.00 eq) was added dropwise until the pH had reached 8 at 25 °C. The mixture was stirred at 25 °C for 0.5 h. AcOH (394 ug, 6.56 μmol, 3.75e-1 μL, 0.0100 eq) was added dropwise until the pH had reached 6 at 25 °C. The mixture was stirred at 25 °C for 0.5 h. NaBH₃CN (124 mg, 1.97 mmol, 3.00 eq) was added to the mixture at 25 °C. The mixture was stirred at 25 °C for 2 h. LCMS showed that the desired mass was detected. The reaction mixture was concentrated under reduced pressure to remove solvent. Water (5 mL) was added to the mixture at 25 °C. Then the reaction mixture was extracted with EtOAc (4 mL*3). The organic layers were dried over anhydrous sodium sulfate, filtered and the filtrate was concentrated under reduced pressure to give a residue. The residue was purified by prep-TLC (SiO₂, Petroleum ether:Ethyl acetate=5:1) to give the title compound (077-2) (300 mg, 451 μmol, 68.8% yield, 83.4% purity) as a white solid. LCMS (ES+): m/z 555.3 [M+H]+.

Synthesis of 3-(1-oxo-5-((1-(2-(piperidin-4-yl)ethyl)piperidin-4-yl)oxy)isoindolin-2-yl)piperidine-2,6-dione (077-3). To a solution of tert-butyl 4-(2-(4-((2-(2,6-dioxopiperidin-3-yl)-1-oxoisoindolin-5-yl)oxy)piperidin-1-yl)ethyl)piperidine-1-carboxylate (077-2) (200 mg, 361 μmol, 1.00 eq) in DCM (3 mL) was added TFA (1 mL) at 25 °C. The mixture was stirred at 25 °C for 1.5 h. LCMS showed that the desired mass was detected. The reaction mixture was concentrated under reduced pressure to remove solvent to give the title compound (077-3) (200 mg, crude, TFA) as a white oil. LCMS (ES+): m/z 455.2 [M+H]+.

Synthesis of cis-3-(5-((1-(2-(1-(3-((4-([1,1′-biphenyl]-3-yl)-5-chloropyrimidin-2-yl)amino)cyclohexane-1-carbonyl)piperidin-4-yl)ethyl)piperidin-4-yl)oxy)-1-oxoisoindolin-2-yl)piperidine-2,6-dione (Compound 79). To a solution of cis-3-((4-([1,1′-biphenyl]-3-yl)-5-chloropyrimidin-2-yl)amino)cyclohexane-1-carboxylic acid (143 mg, 352 μmol, 1.00 eq) (prepared by methods analogous to those shown in Example 22) in DMF (3 mL) were added HOAt (23.9 mg, 176 μmol, 24.6 μL, 0.50 eq), DIEA (227 mg, 1.76 mmol, 306 μL, 5.00 eq), HATU (161 mg, 422 μmol, 1.20 eq) and 3-(1-oxo-5-((1-(2-(piperidin-4-yl)ethyl)piperidin-4-yl)oxy)isoindolin-2-yl)piperidine-2,6-dione (077-3) (200 mg, 352 μmol, 1.00 eq, TFA) at 25 °C. The mixture was stirred at 25 °C for 3 h. LCMS showed that the desired mass was detected. The reaction mixture was filtered and the filtrate was concentrated under reduced pressure to give a residue. The residue were purified by prep-HPLC (column: Phenomenex C18 75*30 mm*3 μm; mobile phase: [water(NH₄HCO₃)-ACN]; B %: 47%-77%, 8 min) to give the title compound (Compound 79) (73.6 mg, 85.09 μmol, 24.2% yield, 97.6% purity) as a white solid. LCMS (ES+): m/z 844.3 [M+H]+.

### Example 78. Synthesis of cis-3-(5-((1-(2-(4-(3-((4-([1,1′-biphenyl]-3-yl)-5-chloropyrimidin-2-yl)amino)cyclohexane-1-carbonyl)piperazin-1-yl)ethyl)piperidin-4-yl)oxy)-1-oxoisoindolin-2-yl)piperidine-2,6-dione (Compound 80)

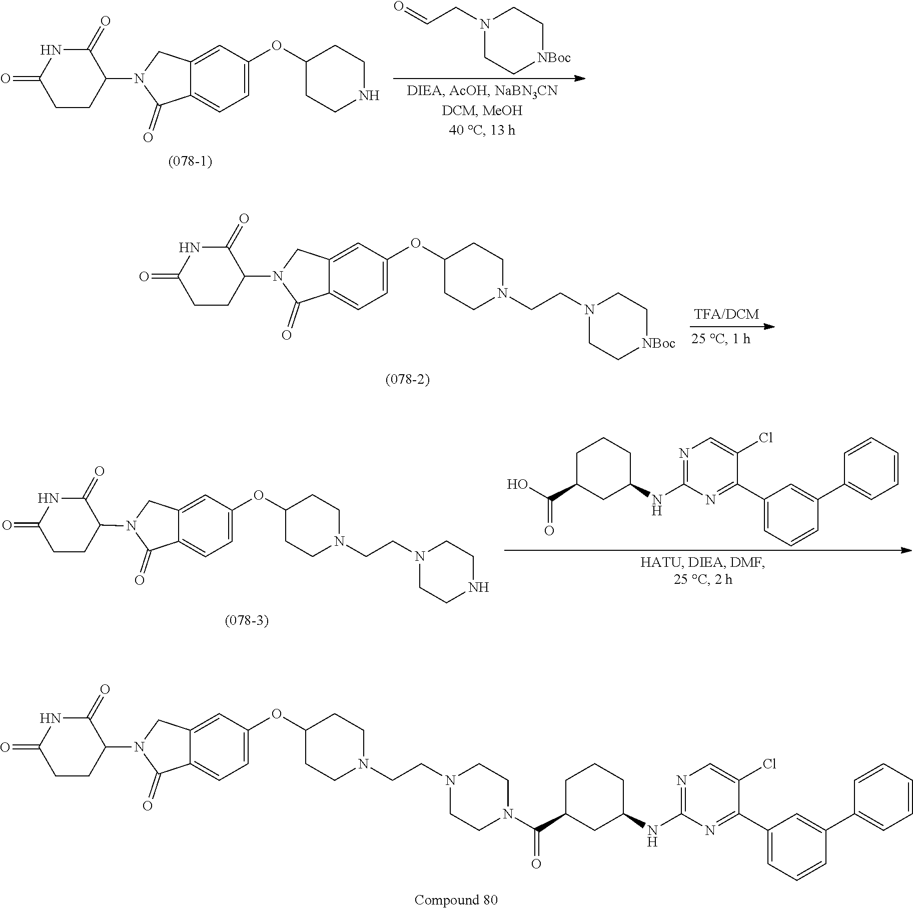 [SMILES: O=C1CCC(N2Cc3cc(OC4CCNCC4)ccc3C2=O)C(=O)N1 ; CC(C)(C)OC(=O)N1CCN(CC=O)CC1 ; CC(C)(C)OC(=O)N1CCN(CCN2CCC(Oc3ccc4c(c3)CN(C3CCC(=O)NC3=O)C4=O)CC2)CC1 ; O=C1CCC(N2Cc3cc(OC4CCN(CCN5CCNCC5)CC4)ccc3C2=O)C(=O)N1 ; O=C(O)[C@H]1CCC[C@@H](Nc2ncc(Cl)c(-c3cccc(-c4ccccc4)c3)n2)C1 ; O=C1CCC(N2Cc3cc(OC4CCN(CCN5CCN(C(=O)[C@H]6CCC[C@@H](Nc7ncc(Cl)c(-c8cccc(-c9ccccc9)c8)n7)C6)CC5)CC4)ccc3C2=O)C(=O)N1]

Synthesis of tert-butyl 4-(2-(4-((2-(2,6-dioxopiperidin-3-yl)-1-oxoisoindolin-5-yl)oxy)piperidin-1-yl)ethyl)piperazine-1-carboxylate (078-2). To a solution of 3-(1-oxo-5-(piperidin-4-yloxy)isoindolin-2-yl)piperidine-2,6-dione (078-1) (600 mg, 1.31 mmol, 1.00 eq, TFA) (prepared as described in Example 83) in DCM (5 mL) and MeOH (10 mL) was added DIEA (170 mg, 1.31 mmol, 228 μL, 1.00 eq) to adjust pH to 8 at 25 °C. The mixture was stirred at 25 °C for 0.5 h. Then to the mixture was added AcOH (7.88 mg, 131 μmol, 7.50 μL, 0.100 eq) to adjust pH to 6 at 25 °C, then the mixture was added tert-butyl 4-(2-oxoethyl)piperazine-1-carboxylate (898 mg, 3.94 mmol, 3.00 eq) at 40 °C for 0.5 h. Then to the mixture was added NaBH₃CN (330 mg, 5.25 mmol, 4.00 eq) at 25 °C. The resulting mixture was stirred at 40 °C for 12 h. LCMS showed 3-(1-oxo-5-(piperidin-4-yloxy)isoindolin-2-yl)piperidine-2,6-dione (078-1) was consumed and the desired mass was detected. H₂O (15 mL) was added to the solution, the mixture was extracted with EtOAc (15 mL*3), the combined organic layers were dried over Na₂SO₄, then concentrated in vacuo. The mixture was purified by prep-TLC (SiO₂, DCM:MeOH=5:1) to give the title compound (078-2) (360 mg, 648 μmol, 49.4% yield) as a white solid. LCMS (ES+): m/z 556.3 [M+H]+.

Synthesis of 3-(1-oxo-5-((1-(2-(piperazin-J-yl)ethyl)piperidin-4-yl)oxy)isoindolin-2-yl)piperidine-2,6-dione (078-3). A solution of tert-butyl 4-(2-(4-((2-(2,6-dioxopiperidin-3-yl)-1-oxoisoindolin-5-yl)oxy)piperidin-1-yl)ethyl)piperazine-1-carboxylate (078-2) (340 mg, 612 μmol, 1.00 eq) in DCM (5 mL) and TFA (1 mL) was stirred at 25 °C for 1 h. LCMS showed tert-butyl 4-(2-(4-((2-(2,6-dioxopiperidin-3-yl)-1-oxoisoindolin-5-yl)oxy)piperidin-1-yl)ethyl)piperazine-1-carboxylate (078-2) was consumed and the desired mass was detected. The mixture was concentrated in vacuo to give the title compound (078-3) (270 mg, crude, TFA) as a yellow oil. LCMS (ES+): m/z 456.2 [M+H]+.

Synthesis of cis-3-(5-((1-(2-(4-(3-((4-([1,1′-biphenyl]-3-yl)-5-chloropyrimidin-2-yl)amino)cyclohexane-1-carbonyl)piperazin-1-yl)ethyl)piperidin-4-yl)oxy)-1-oxoisoindolin-2-yl)piperidine-2,6-dione (Compound 80). To a solution of cis-3-((4-([1,1′-biphenyl]-3-yl)-5-chloropyrimidin-2-yl)amino)cyclohexane-1-carboxylic acid (179 mg, 439 μmol, 1.00 eq) in DMF (2 mL) were added HATU (200 mg, 527 μmol, 1.20 eq) and DIEA (227 mg, 1.76 mmol, 306 μL, 4.00 eq) at 25 °C, then 3-(1-oxo-5-((1-(2-(piperazin-1-yl)ethyl)piperidin-4-yl)oxy)isoindolin-2-yl)piperidine-2,6-dione (078-3) (250 mg, 439 μmol, 1.00 eq, TFA) was added to the mixture, the mixture was stirred at 25 °C for 2 h. LCMS showed 3-(1-oxo-5-((1-(2-(piperazin-1-yl)ethyl)piperidin-4-yl)oxy)isoindolin-2-yl)piperidine-2,6-dione (078-3) was consumed and the desired mass was detected. The mixture was concentrated in vacuo. The mixture was purified by prep-HPLC (column: Waters Xbridge BEH C18 100*30 mm*10 μm; mobile phase: [water (NH₄HCO₃)-ACN]; B %: 45%-80%, 8 min) to give the title compound (Compound 80) (138 mg, 158 μmol, 36.1% yield, 96.8% purity) as a white solid. LCMS (ES+): m/z 845.4 [M+H]+.

### Example 79. Synthesis of cis-3-(5-((1-(2-(1-(3-((4-([1,1′-biphenyl]-3-yl)-5-chloropyrimidin-2-yl)amino)cyclohexane-1-carbonyl)piperidin-4-yl)acetyl)piperidin-4-yl)oxy)-1-oxoisoindolin-2-yl)piperidine-2,6-dione (Compound 81)

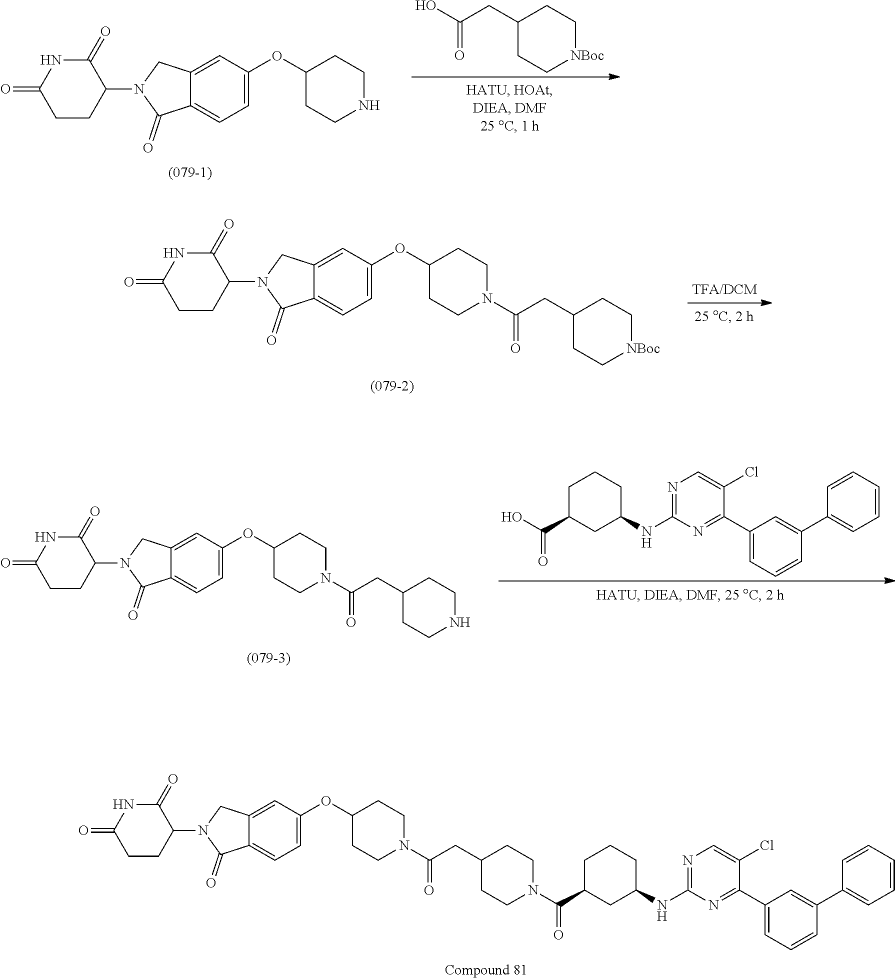 [SMILES: O=C1CCC(N2Cc3cc(OC4CCNCC4)ccc3C2=O)C(=O)N1 ; CC(C)(C)OC(=O)N1CCC(CC(=O)O)CC1 ; CC(C)(C)OC(=O)N1CCC(CC(=O)N2CCC(Oc3ccc4c(c3)CN(C3CCC(=O)NC3=O)C4=O)CC2)CC1 ; O=C1CCC(N2Cc3cc(OC4CCN(C(=O)CC5CCNCC5)CC4)ccc3C2=O)C(=O)N1 ; O=C(O)[C@H]1CCC[C@@H](Nc2ncc(Cl)c(-c3cccc(-c4ccccc4)c3)n2)C1 ; O=C1CCC(N2Cc3cc(OC4CCN(C(=O)CC5CCN(C(=O)[C@H]6CCC[C@@H](Nc7ncc(Cl)c(-c8cccc(-c9ccccc9)c8)n7)C6)CC5)CC4)ccc3C2=O)C(=O)N1]

Synthesis of tert-butyl 4-(2-(4-((2-(2, 6-dioxopiperidin-3-yl)-1-oxoisoindolin-5-yl) oxy) piperidin-1-yl)-2-oxoethyl) piperidine-1-carboxylate (079-2). To a solution of 2-(1-(tert-butoxycarbonyl)piperidin-4-yl)acetic acid (255 mg, 1.05 mmol, 1.20 eq) in DMF (10 mL) were added DIPEA (565 mg, 4.37 mmol, 762 μL, 5.00 eq), HATU (499 mg, 1.31 mmol, 1.50 eq) and HOAt (59.5 mg, 437 μmol, 61.2 μL, 0.500 eq) at 25 °C, then added 3-(1-oxo-5-(piperidin-4-yloxy)isoindolin-2-yl)piperidine-2,6-dione (079-1) (400 mg, 875 μmol, 1.00 eq, TFA) (prepared as described in Example 83). The mixture was stirred at 25 °C for 1 h. LCMS showed that 3-(1-oxo-5-(piperidin-4-yloxy)isoindolin-2-yl)piperidine-2,6-dione (079-1) was consumed completely the desired mass was detected. H₂O (10 mL) was added to the solution, then the mixture was extracted with EtOAc (10 mL*3), the combined organic layers were dried over Na₂SO₄, then concentrated in vacuo. The mixture was purified by MPLC (SiO₂, Dichloromethane:Methanol=1:0 to 1:1) to give the title compound (079-2) (490 mg, 862 μmol, 98.5% yield) as a yellow solid. LCMS (ES+): m/z 469.2 [M+H−100]+.

Synthesis of 3-(1-oxo-5-((1-(2-(piperidin-4-yl)acetyl)piperidin-4-yl)oxy)isoindolin-2-yl)piperidine-2,6-dione (079-3). To a solution tert-butyl 4-(2-(4-((2-(2, 6-dioxopiperidin-3-yl)-1-oxoisoindolin-5-yl) oxy) piperidin-1-yl)-2-oxoethyl) piperidine-1-carboxylate (079-2) (480 mg, 844 μmol, 1.00 eq) in DCM (7 mL) was added TFA (2 mL) at 25 °C. The mixture was stirred at 20 °C for 2 h. LCMS showed that tert-butyl 4-(2-(4-((2-(2, 6-dioxopiperidin-3-yl)-1-oxoisoindolin-5-yl) oxy) piperidin-1-yl)-2-oxoethyl) piperidine-1-carboxylate (079-2) was consumed completely and the desired mass was detected. The mixture was concentrated in vacuo to give the title compound (079-3) (520 mg, crude, TFA) as a yellow oil. LCMS (ES+): m/z 469.2 [M+H]+.

Synthesis of cis-3-(5-((1-(2-(1-(3-((4-([1,1′-biphenyl]-3-yl)-5-chloropyrimidin-2-yl)amino)cyclohexane-1-carbonyl)piperidin-4-yl)acetyl)piperidin-4-yl)oxy)-1-oxoisoindolin-2-yl)piperidine-2,6-dione (Compound 81). To a solution of cis-3-((4-([1,1′-biphenyl]-3-yl)-5-chloropyrimidin-2-yl)amino)cyclohexane-1-carboxylic acid (420 mg, 1.03 mmol, 1.20 eq) in DMF (10 mL) were added HOAt (72.6 mg, 534 μmol, 74.6 μL, 0.500 eq), HATU (609 mg, 1.60 mmol, 1.50 eq) and DIPEA (690 mg, 5.34 mmol, 929 μL, 5.00 eq) at 25 °C, then 3-(1-oxo-5-((1-(2-(piperidin-4-yl)acetyl)piperidin-4-yl)oxy)isoindolin-2-yl)piperidine-2,6-dione (079-3) (500 mg, 858 μmol, 1.00 eq, TFA) was added to the mixture, the mixture was stirred at 25 °C for 2 h. LCMS showed that 3-(1-oxo-5-((1-(2-(piperidin-4-yl)acetyl)piperidin-4-yl)oxy)isoindolin-2-yl)piperidine-2,6-dione (079-3) was consumed completely and the desired mass was detected. H₂O (10 mL) was added to the solution, then the mixture was extracted with EtOAc (10 mL*3), the combined organic layers were dried over Na₂SO₄, then concentrated in vacuo. The residue was purified by prep-HPLC (column: Waters Xbridge BEH C18 100*30 mm*10 μm; mobile phase: [water (NH₄HCO₃)-ACN]; B %: 50%-80%, 8 min) to give the title compound (Compound 81) (76.7 mg, 81.39 μmol, 9.48% yield, 91.1% purity) as a white solid. LCMS (ES+): m/z 858.4 [M+H]+.

### Example 80. Synthesis of cis-3-(5-((1-(2-(4-(3-((4-([1,1′-biphenyl]-3-yl)-5-chloropyrimidin-2-yl)amino)cyclohexane-1-carbonyl)piperazin-1-yl)acetyl)piperidin-4-yl)oxy)-1-oxoisoindolin-2-yl)piperidine-2,6-dione (Compound 82)

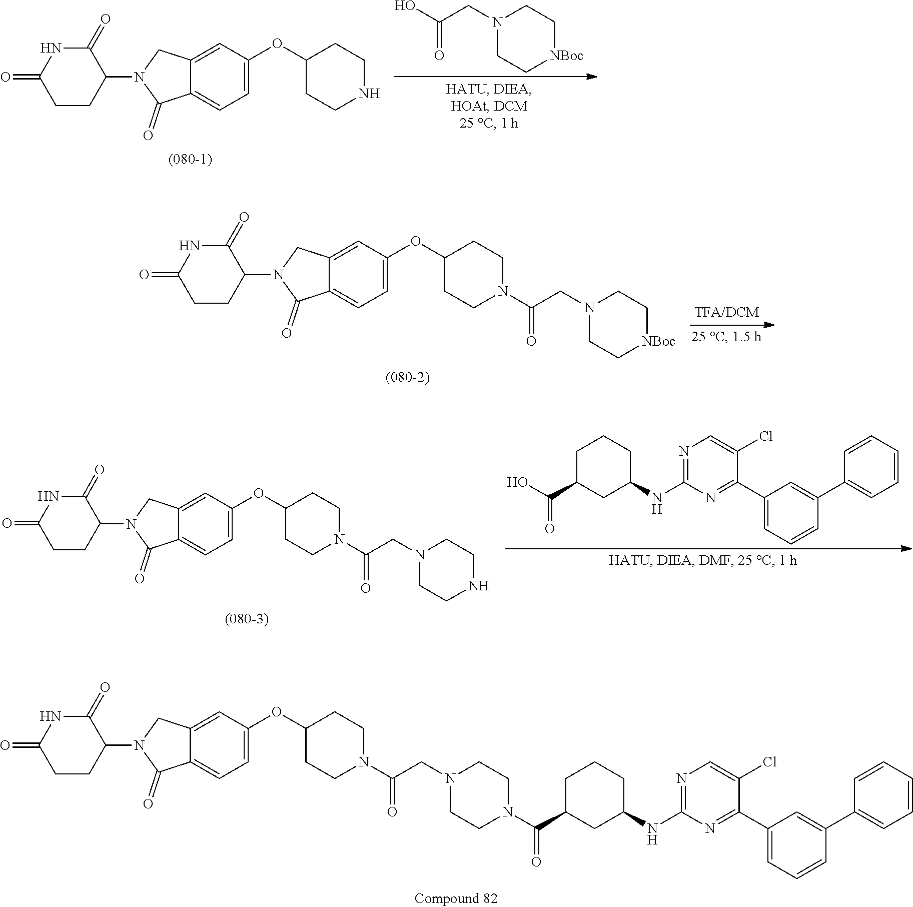 [SMILES: O=C1CCC(N2Cc3cc(OC4CCNCC4)ccc3C2=O)C(=O)N1 ; CC(C)(C)OC(=O)N1CCN(CC(=O)O)CC1 ; CC(C)(C)OC(=O)N1CCN(CC(=O)N2CCC(Oc3ccc4c(c3)CN(C3CCC(=O)NC3=O)C4=O)CC2)CC1 ; O=C1CCC(N2Cc3cc(OC4CCN(C(=O)CN5CCNCC5)CC4)ccc3C2=O)C(=O)N1 ; O=C(O)[C@H]1CCC[C@@H](Nc2ncc(Cl)c(-c3cccc(-c4ccccc4)c3)n2)C1 ; O=C1CCC(N2Cc3cc(OC4CCN(C(=O)CN5CCN(C(=O)[C@H]6CCC[C@@H](Nc7ncc(Cl)c(-c8cccc(-c9ccccc9)c8)n7)C6)CC5)CC4)ccc3C2=O)C(=O)N1]

Synthesis of tert-butyl 4-(2-(4-((2-(2,6-dioxopiperidin-3-yl)-1-oxoisoindolin-5-yl)oxy)piperidin-1-yl)-2-oxoethyl)piperazine-1-carboxylate (080-2). To a solution of 2-(4-tert-butoxycarbonylpiperazin-1-yl)acetic acid (128 mg, 525 μmol, 1.20 eq) in DCM (5 mL) were added HATU (249 mg, 656 μmol, 1.50 eq), HOAt (29.8 mg, 219 μmol, 30.6 μL, 0.500 eq), 3-(1-oxo-5-(piperidin-4-yloxy)isoindolin-2-yl)piperidine-2,6-dione (080-1) (200 mg, 437 μmol, 1.00 eq, TFA) (prepared as described in Example 83) and DIPEA (283 mg, 2.19 mmol, 381 μL, 5.00 eq) at 25 °C. The mixture was stirred at 25 °C for 1 h. LCMS showed 3-(1-oxo-5-(piperidin-4-yloxy)isoindolin-2-yl)piperidine-2,6-dione (080-1) was consumed completely and the desired mass was detected. The reaction mixture was concentrated under reduced pressure to remove solvent. The residue was purified by prep-TLC (SiO₂, Petroleum ether:Ethyl acetate=0:1) to give the title compound (080-2) (150 mg, 263 μmol, 60.2% yield) as a yellow oil. LCMS (ES+): m/z 570.3 [M+H]+.

Synthesis of 3-(1-oxo-5-((1-(2-(piperazin-1-yl)acetyl)piperidin-4-yl)oxy)isoindolin-2-yl)piperidine-2,6-dione (080-3). To a solution of tert-butyl 4-(2-(4-((2-(2,6-dioxopiperidin-3-yl)-1-oxoisoindolin-5-yl)oxy)piperidin-1-yl)-2-oxoethyl)piperazine-1-carboxylate (080-2) (140 mg, 246 μmol, 1.00 eq) in DCM (3 mL) was added TFA (1 mL) at 25 °C. The mixture was stirred at 25 °C for 1 h. LCMS showed ~30.4% of tert-butyl 4-(2-(4-((2-(2,6-dioxopiperidin-3-yl)-1-oxoisoindolin-5-yl)oxy)piperidin-1-yl)-2-oxoethyl)piperazine-1-carboxylate (080-2) was remaining and desired mass was detected. Then the mixture was stirred at 25 °C for 0.5 h. LCMS showed tert-butyl 4-(2-(4-((2-(2,6-dioxopiperidin-3-yl)-1-oxoisoindolin-5-yl)oxy)piperidin-1-yl)-2-oxoethyl)piperazine-1-carboxylate (080-2) was consumed completely and the desired mass was detected. The reaction mixture was concentrated under reduced pressure to remove solvent to give the title compound (080-3) (140 mg, crude, TFA) as a yellow oil. LCMS (ES+): m/z 470.2 [M+H]+.

Synthesis of cis-3-(5-((1-(2-(4-(3-((4-([1,1′-biphenyl]-3-yl)-5-chloropyrimidin-2-yl)amino)cyclohexane-1-carbonyl)piperazin-1-yl)acetyl)piperidin-4-yl)oxy)-1-oxoisoindolin-2-yl)piperidine-2,6-dione (Compound 82). To a solution of 3-(1-oxo-5-((1-(2-(piperazin-1-yl)acetyl)piperidin-4-yl)oxy)isoindolin-2-yl)piperidine-2,6-dione (080-3) (140 mg, 240 μmol, 1.00 eq, TFA) in DMF (3 mL) were added HATU (109 mg, 288 μmol, 1.20 eq), cis-3-((4-([1,1′-biphenyl]-3-yl)-5-chloropyrimidin-2-yl)amino)cyclohexane-1-carboxylic acid (97.9 mg, 240 μmol, 1.00 eq) and DIPEA (155 mg, 1.20 mmol, 209 μL, 5.00 eq) at 25 °C. The mixture was stirred at 25 °C for 1 h. LCMS showed 3-(1-oxo-5-((1-(2-(piperazin-1-yl)acetyl)piperidin-4-yl)oxy)isoindolin-2-yl)piperidine-2,6-dione (080-3) was consumed completely and the desired mass was detected. The organic layers were filtered and the filtrate was concentrated under reduced pressure to give a residue. The residue was purified by prep-HPLC (column: Phenomenex Luna 80*30 mm*3 μm; mobile phase: [water(FA)-ACN]; B %: 25%-75%, 8 min) to give the title compound (Compound 82) (31.0 mg, 34.6 μmol, 14.4% yield, 95.8% purity) as a white solid. LCMS (ES+): m/z 859.2 [M+H]+.

### Example 81. Synthesis of 3-(5-((1-(2-(4-(4-((4-([1,1′-biphenyl]-3-yl)-5-chloropyrimidin-2-yl)amino)piperidine-1-carbonyl)piperidin-1-yl)acetyl)piperidin-4-yl)oxy)-1-oxoisoindolin-2-yl)piperidine-2,6-dione (Compound 83)

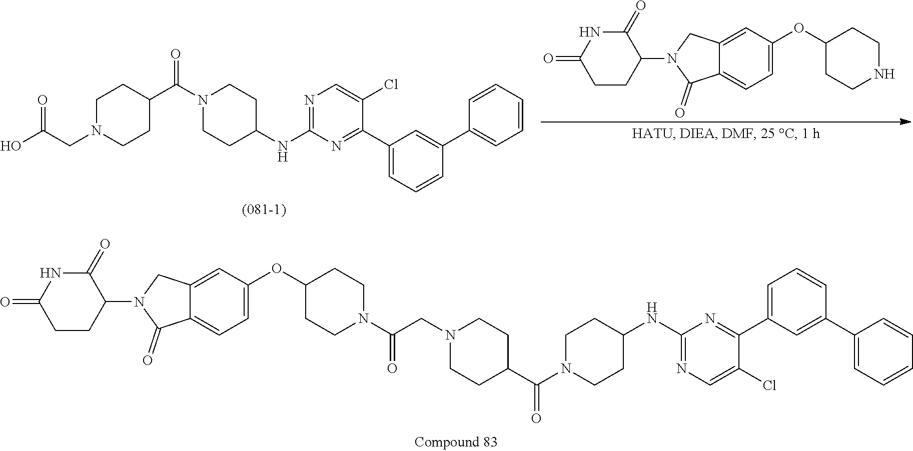 [SMILES: O=C(O)CN1CCC(C(=O)N2CCC(Nc3ncc(Cl)c(-c4cccc(-c5ccccc5)c4)n3)CC2)CC1 ; O=C1CCC(N2Cc3cc(OC4CCNCC4)ccc3C2=O)C(=O)N1 ; O=C1CCC(N2Cc3cc(OC4CCN(C(=O)CN5CCC(C(=O)N6CCC(Nc7ncc(Cl)c(-c8cccc(-c9ccccc9)c8)n7)CC6)CC5)CC4)ccc3C2=O)C(=O)N1]

Synthesis of 3-(5-((1-(2-(4-(4-((4-([1,1′-biphenyl]-3-yl)-5-chloropyrimidin-2-yl)amino)piperidine-1-carbonyl)piperidin-1-yl)acetyl)piperidin-4-yl)oxy)-1-oxoisoindolin-2-yl)piperidine-2,6-dione (Compound 83). To a solution of 3-(1-oxo-5-(piperidin-4-yloxy)isoindolin-2-yl)piperidine-2,6-dione (102 mg, 299 μmol, 2.00 eq) (prepared as described in Example 83) and 2-(4-(4-((4-([1,1′-biphenyl]-3-yl)-5-chloropyrimidin-2-yl)amino)piperidine-1-carbonyl)piperidin-1-yl)acetic acid (081-1) (80.0 mg, 149 μmol, 1.00 eq) in DMF (5.00 mL) were added HATU (56.9 mg, 149 μmol, 1.00 eq) and DIEA (58.0 mg, 449 μmol, 78.2 μL, 3.00 eq) at 25 °C. The mixture was stirred at 25 °C for 1 h. LCMS showed 2-(4-(4-((4-([1,1′-biphenyl]-3-yl)-5-chloropyrimidin-2-yl)amino)piperidine-1-carbonyl)piperidin-1-yl)acetic acid (081-1) was consumed completely and the desired mass was detected. The reaction mixture was diluted with H₂O 20.0 mL and extracted with EtOAc (20.0 mL*3). The combined organic layers were dried over Na₂SO₄, filtered and concentrated under reduced pressure. The residue was purified by prep-HPLC (column: Phenomenex Luna C18 150*30 mm*5 μm; mobile phase: [water(FA)-ACN]; B %: 30%-60%, 8 min) to give the title compound (Compound 83) (23.8 mg, 27.3 μmol, 18.2% yield, 98.9% purity) as a white solid. LCMS (ES+): m/z 859.6 [M+H]+.

### Example 82. Synthesis of 3-((4-(4-(4-(3-(4-((4-([1,1′-biphenyl]-3-yl)-5-chloropyrimidin-2-yl)amino)piperidin-1-yl)-3-oxopropyl)piperazin-1-yl)piperidin-1-yl)phenyl)amino)piperidine-2,6-dione (Compound 84)

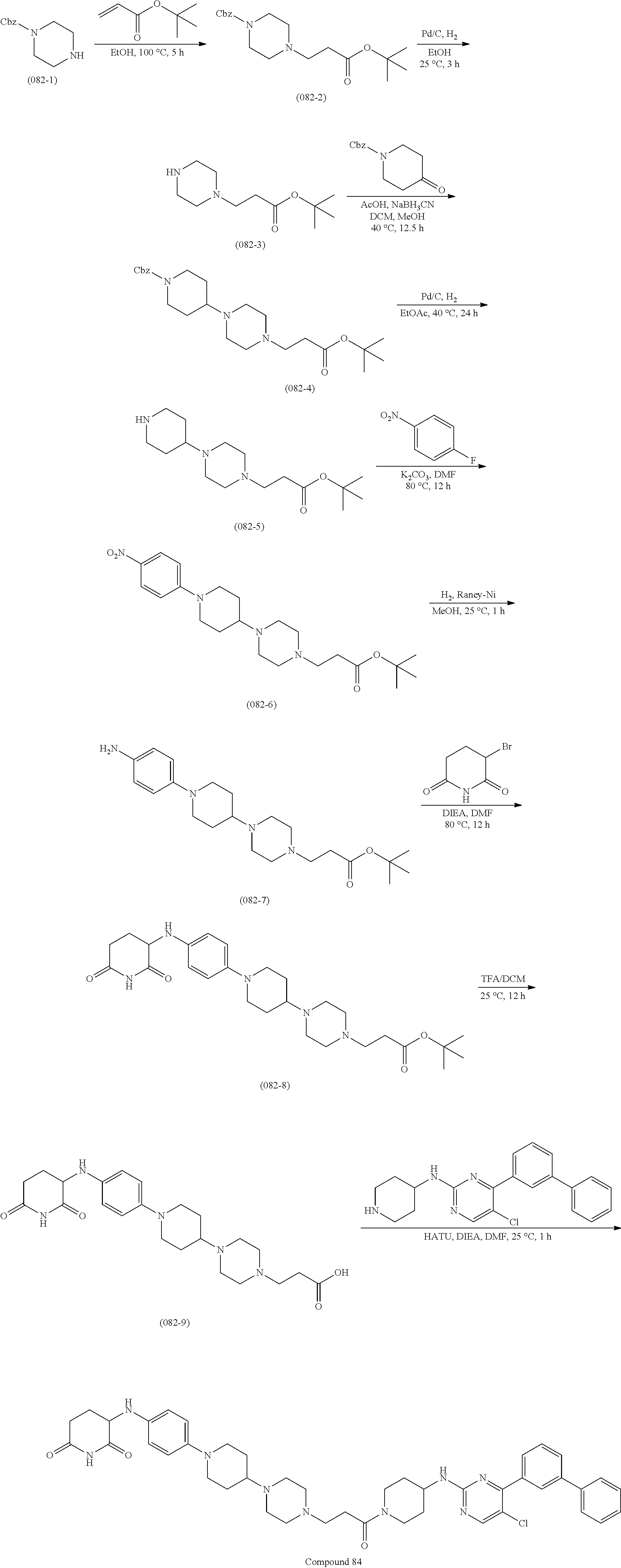

Synthesis of benzyl 4-(3-(tert-butoxy)-3-oxopropyl)piperazine-1-carboxylate (082-2). To a solution of benzyl piperazine-1-carboxylate (082-1) (6.46 g, 29.3 mmol, 5.66 mL, 0.751 eq) in EtOH (20 mL) was added tert-butyl prop-2-enoate (5.00 g, 39.0 mmol, 5.66 mL, 1.00 eq) at 25 °C. The mixture was stirred at 100 °C for 5 h. LCMS showed the desired mass was detected. Petroleum ether (33 mL), H₂O (18 mL) and HCl (1M, 33 mL) were added to the mixture at 25 °C, and the aqueous layer were separated. Ethyl acetate (170 mL) and NaOH (1 M, 33 mL) were added to the aqueous layer, the organic was separated. The organic layer was dried over anhydrous sodium sulfate, filtered and the filtrate was concentrated under reduced pressure to give the title compound (082-2) (10 g, crude) as a brown oil. LCMS (ES+): m/z 349.1 [M+H]+.

Synthesis of tert-butyl 3-(piperazin-1-yl)propanoate (082-3). 10% Pd/C (3.00 g, 50% purity) was added to EtOH (100 mL) at 25 °C under N₂ atmosphere, then benzyl 4-(3-(tert-butoxy)-3-oxopropyl)piperazine-1-carboxylate (082-2) (10.0 g, 28.7 mmol, 1.00 eq) was added to the mixture at 25 °C, the suspension was degassed and purged with H₂ for three times. The mixture was stirred at 25 °C for 3 h under H₂ (15 psi) atmosphere. LCMS showed benzyl 4-(3-(tert-butoxy)-3-oxopropyl)piperazine-1-carboxylate (082-2) was consumed completely and the desired mass was detected. The mixture was filtered and the filtrate was concentrated under reduced pressure to give the title compound (082-3) (5.80 g, crude) as a yellow oil. LCMS (ES+): m/z 215.2 [M+H]+.

Synthesis of benzyl 4-(4-(3-(tert-butoxy)-3-oxopropyl)piperazin-1-yl)piperidine-1-carboxylate (082-4). To a solution of tert-butyl 3-(piperazin-1-yl)propanoate (082-3) (5.80 g, 27.1 mmol, 1.00 eq) in DCM (30 mL) and MeOH (30 mL) were added AcOH (163 mg, 2.71 mmol, 155 μL, 0.100 eq) and benzyl 4-oxopiperidine-1-carboxylate (44.2 g, 190 mmol, 37.8 mL, 7.00 eq) at 25 °C. The mixture was stirred at 40 °C for 0.5 h. Then NaBH₃CN (11.9 g, 190 mmol, 7.00 eq) was added to the mixture at 40 °C. The mixture was stirred at 40 °C for 12 h. LCMS showed tert-butyl 3-(piperazin-1-yl)propanoate (082-3) was consumed completely and the desired mass was detected. Water (20 mL) was added to the mixture at 25 °C. Then the reaction mixture was extracted with DCM (20 mL*3). The organic layers were dried over anhydrous sodium sulfate, filtered and the filtrate was concentrated under reduced pressure to give a residue. The residue was purified by column chromatography (SiO₂, Petroleum ether/Ethyl acetate=10/1 to 1/1) to give the title compound (082-4) (6.00 g, 13.9 mmol, 51.4% yield) as a yellow oil. LCMS (ES+): m/z 432.3 [M+H]+.

Synthesis of tert-butyl 3-(4-(piperidin-4-yl)piperazin-1-yl)propanoate (082-5). 10% Pd/C (16.0 g, 50.0% purity) was added to EtOAc (100 mL) at 25 °C under N₂ atmosphere, then benzyl 4-(4-(3-(tert-butoxy)-3-oxopropyl)piperazin-1-yl)piperidine-1-carboxylate (082-4) (5.00 g, 11.6 mmol, 1.00 eq) was added to the mixture at 25 °C, the suspension was degassed and purged with H₂ for three times. The mixture was stirred at 40 °C for 12 h under H₂ (15 psi) atmosphere. LCMS showed ~34% of benzyl 4-(4-(3-(tert-butoxy)-3-oxopropyl)piperazin-1-yl)piperidine-1-carboxylate (082-4) was remaining, and ~26% of the desired mass was detected. The reaction mixture was filtered and the filtrate was concentrated under reduced pressure to give a residue. Then 10% Pd/C (40.0 g, 50% purity) was added to the mixture at 25 °C. The mixture was stirred at 40 °C for 12 h. LCMS showed benzyl 4-(4-(3-(tert-butoxy)-3-oxopropyl)piperazin-1-yl)piperidine-1-carboxylate (082-4) was consumed completely and the desired mass was detected. The reaction mixture was filtered and the filtrate was concentrated under reduced pressure to give the title compound (082-5) (4.60 g, 11.2 mmol, 96.1% yield, 72.0% purity) as a yellow oil. LCMS (ES+): m/z 298.3 [M+H]+.

Synthesis of tert-butyl 3-(4-(1-(4-nitrophenyl)piperidin-4-yl)piperazin-1-yl)propanoate (082-6). To a solution of 1-fluoro-4-nitro-benzene (949 mg, 6.72 mmol, 713 L, 1.00 eq) in DMF (10 mL) were added tert-butyl 3-(4-(piperidin-4-yl)piperazin-1-yl)propanoate (082-5) (2.00 g, 6.72 mmol, 1.00 eq) and K₂CO₃ (2.79 g, 20.1 mmol, 3.00 eq) at 25 °C. The mixture was stirred at 80 °C for 12 h. LCMS showed tert-butyl 3-(4-(piperidin-4-yl)piperazin-1-yl)propanoate (082-5) was consumed completely and the desired mass was detected. Water (10 mL) was added to the mixture at 25 °C. Then the reaction mixture was extracted with EtOAc (10 mL*3). The organic layers were dried over anhydrous sodium sulfate, filtered and the filtrate was concentrated under reduced pressure to give a residue. The residue was purified by column chromatography (SiO₂, Ethyl acetate/Methanol=1/0 to 10/1) to give the title compound (082-6) (1.10 g, 2.63 mmol, 39.1% yield) as a yellow solid. LCMS (ES+): m/z 419.3 [M+H]+.

Synthesis of tert-butyl 3-(4-(1-(4-aminophenyl)piperidin-4-yl)piperazin-1-yl)propanoate (082-7). Raney-Ni (2.00 g) was added to MeOH (10 mL) at 25 °C under N₂ atmosphere, then tert-butyl 3-(4-(1-(4-nitrophenyl)piperidin-4-yl)piperazin-1-yl)propanoate (082-6) (1.00 g, 2.39 mmol, 1.00 eq) was added to the mixture at 25 °C, the suspension was degassed and purged with H₂ for three times. The mixture was stirred at 25 °C for 1 h under H₂ (15 psi) atmosphere. LCMS showed tert-butyl 3-(4-(1-(4-nitrophenyl)piperidin-4-yl)piperazin-1-yl)propanoate (082-6) was consumed completely and the desired mass was detected. The reaction mixture was filtered and the filtrate was concentrated under reduced pressure to give the title compound (082-7) (1.00 g, crude) as a black solid. LCMS (ES+): m/z 389.3 [M+H]+.

Synthesis of tert-butyl 3-(4-(1-(4-((2,6-dioxopiperidin-3-yl)amino)phenyl)piperidin-4-yl)piperazin-1-yl)propanoate (082-8). To a solution of tert-butyl 3-(4-(1-(4-aminophenyl)piperidin-4-yl)piperazin-1-yl)propanoate (082-7) (750 mg, 1.93 mmol, 1.00 eq) in DMF (10 mL) were added 3-bromopiperidine-2,6-dione (1.11 g, 5.79 mmol, 3.00 eq) and DIEA (748 mg, 5.79 mmol, 1.01 mL, 3.00 eq) at 25 °C. The mixture was stirred at 80 °C for 12 h. LCMS showed tert-butyl 3-(4-(1-(4-aminophenyl)piperidin-4-yl)piperazin-1-yl)propanoate (082-7) was consumed completely and the desired mass was detected. Water (10 mL) was added to the mixture at 25 °C. Then the reaction mixture was extracted with EtOAc (5 mL*3). The organic layers were dried over anhydrous sodium sulfate, filtered and the filtrate was concentrated under reduced pressure to give a residue. The residue was purified by column chromatography (SiO₂, Petroleum ether/Ethyl acetate=10/1 to 1/1) to give the title compound (082-8) (900 mg, 1.80 mmol, 93.3% yield) as a black solid. LCMS (ES+): m/z 500.4 [M+H]+.

Synthesis of 3-(4-(1-(4-((2,6-dioxopiperidin-3-yl)amino)phenyl)piperidin-4-yl)piperazin-1-yl)propanoic acid (082-9). A solution of tert-butyl 3-(4-(1-(4-((2,6-dioxopiperidin-3-yl)amino)phenyl)piperidin-4-yl)piperazin-1-yl)propanoate (082-8) (200 mg, 400 μmol, 1 eq) in TFA (2 mL) and DCM (6 mL) was added stirred at 25 °C for 12 h. LCMS showed tert-butyl 3-(4-(1-(4-((2,6-dioxopiperidin-3-yl)amino)phenyl)piperidin-4-yl)piperazin-1-yl)propanoate (082-8) was consumed completely and the desired mass was detected. The reaction mixture was concentrated under reduced pressure to remove solvent to give the title compound (082-9) (177 mg, crude) as a brown oil. LCMS (ES+): m/z 444.3 [M+H]+.

Synthesis of 3-((4-(4-(4-(3-(4-((4-([1,1′-biphenyl]-3-yl)-5-chloropyrimidin-2-yl)amino)piperidin-1-yl)-3-oxopropyl)piperazin-1-yl)piperidin-1-yl)phenyl)amino)piperidine-2,6-dione (Compound 84). To a solution of 3-(4-(1-(4-((2,6-dioxopiperidin-3-yl)amino)phenyl)piperidin-4-yl)piperazin-1-yl)propanoic acid (082-9) (177 mg, 399 μmol, 1.00 eq) in DMF (2 mL) were added 5-chloro-4-(3-phenylphenyl)-N-(4-piperidyl)pyrimidin-2-amine (Compound B) (191 mg, 399 μmol, 1.00 eq, TFA), HATU (182 mg, 479 μmol, 1.20 eq) and DIEA (155 mg, 1.20 mmol, 209 μL, 3.00 eq) at 25 °C. The mixture was stirred at 25 °C for 1 h. LCMS showed 3-(4-(1-(4-((2,6-dioxopiperidin-3-yl)amino)phenyl)piperidin-4-yl)piperazin-1-yl)propanoic acid (082-9) was consumed completely and the desired mass was detected. The reaction mixture was concentrated under reduced pressure to remove solvent. The residue was purified by prep-HPLC (column: C18-6 100*30 mm*5 μm; mobile phase: [water(FA)-ACN]; B %: 40%-70%, 8 min) to give the title compound (Compound 84) (44.6 mg, 55.5 μmol, 13.9% yield, 98.3% purity) as a purple solid. LCMS (ES+): m/z 790.3 [M+H]+.

### Example 83. Synthesis of 3-(5-((1-((1-(2-(4-((4-([1,1′-biphenyl]-3-yl)-5-chloropyrimidin-2-yl)amino)piperidin-1-yl)-2-oxoethyl)piperidin-4-yl)methyl)piperidin-4-yl)oxy)-1-oxoisoindolin-2-yl)piperidine-2,6-dione (Compound 85)

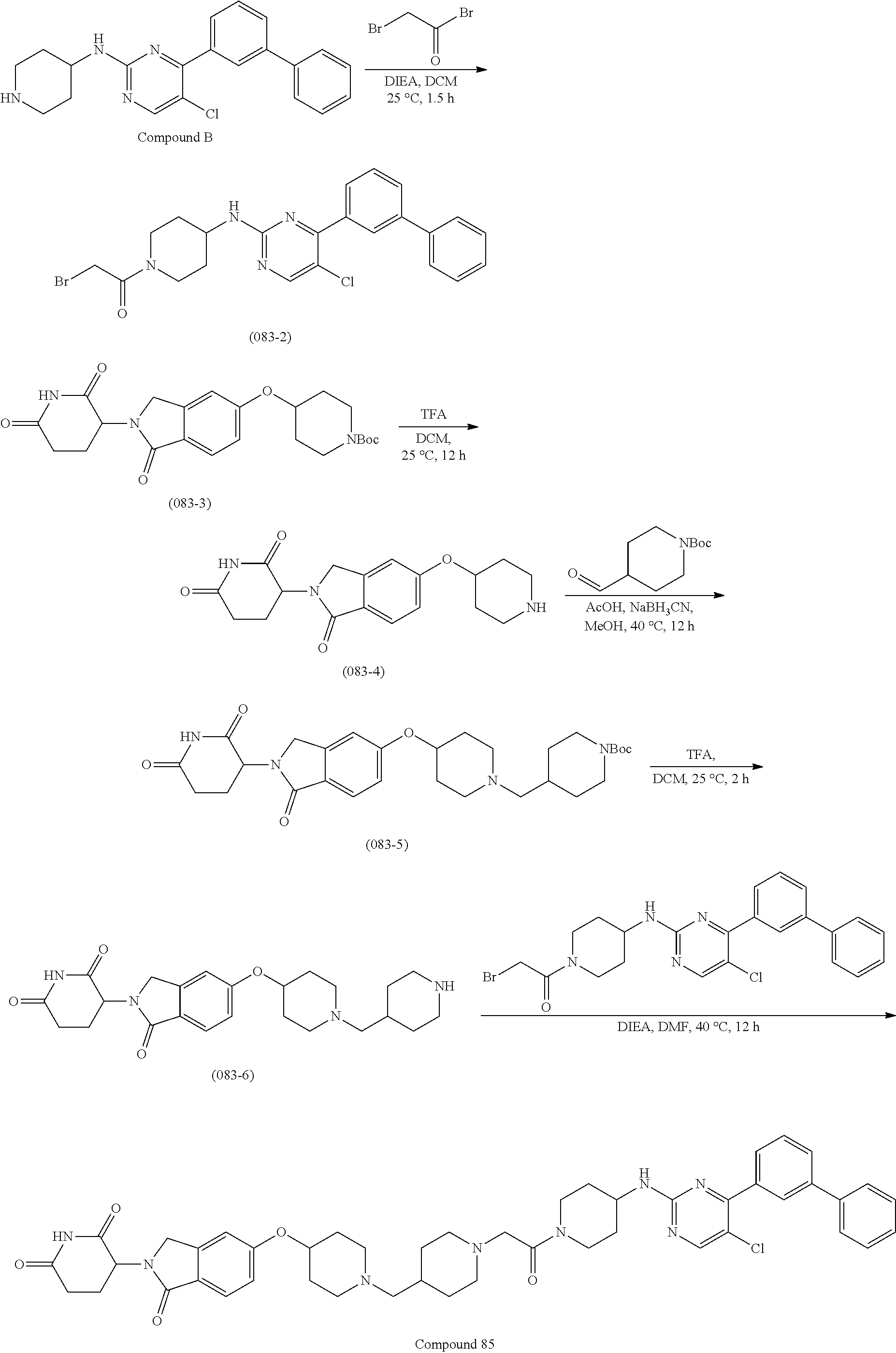

Synthesis of 1-(4-((4-([1,1′-biphenyl]-3-yl)-5-chloropyrimidin-2-yl)amino)piperidin-1-yl)-2-bromoethan-1-one (083-2). To a solution of 4-([1,1′-biphenyl]-3-yl)-5-chloro-N-(piperidin-4-yl)pyrimidin-2-amine (Compound B) (1.27 g, 3.48 mmol, 1.00 eq) in DCM (20 mL) were added 2-bromoacetyl bromide (843 mg, 4.18 mmol, 363 μL, 1.20 eq) and DIPEA (1.35 g, 10.4 mmol, 1.82 mL, 3.00 eq) at 25 °C. The mixture was stirred at 25 °C for 1.5 h. LCMS showed that 4-([1,1′-biphenyl]-3-yl)-5-chloro-N-(piperidin-4-yl)pyrimidin-2-amine (Compound B) was consumed completely and the desired mass was detected. The reaction mixture was quenched by addition H₂O 20 ml at 25 °C, and the aqueous was extracted with DCM (15 mL*3). The combined organic layers were washed with brine 20 mL, dried over Na₂SO₄, filtered and the filtrate was concentrated under reduced pressure to give the title compound (082-2) (1.00 g, 1.37 mmol, 39.3% yield, 66.5% purity) as a yellow solid. LCMS (ES+): m/z 485.2 [M+H]+.

Synthesis of 3-(I-oxo-5-(piperidin-4-yloxy)isoindolin-2-yl)piperidine-2,6-dione (083-4). To a solution of tert-butyl 4-((2-(2,6-dioxopiperidin-3-yl)-1-oxoisoindolin-5-yl)oxy)piperidine-1-carboxylate (8.00 g, 18.0 mmol, 1.00 eq) in DCM (50 mL) was added TFA (23.1 g, 202 mmol, 15.0 mL, 11.2 eq) at 25 °C. The mixture was stirred at 25 °C for 12 h. LCMS showed that tert-butyl 4-((2-(2,6-dioxopiperidin-3-yl)-1-oxoisoindolin-5-yl)oxy)piperidine-1-carboxylate (083-3) was consumed completely and the desired mass was detected. The reaction mixture was concentrated under reduced pressure to give the title compound (083-4) (12.5 g, crude) as a yellow oil. LCMS (ES+): m/z 344.1 [M+H]+.

Synthesis of tert-butyl 4-((4-((2-(2,6-dioxopiperidin-3-yl)-1-oxoisoindolin-5-yl)oxy)piperidin-1-yl)methyl)piperidine-1-carboxylate (083-5). To a solution of 3-(1-oxo-5-(piperidin-4-yloxy)isoindolin-2-yl)piperidine-2,6-dione (083-4) (3.00 g, 8.74 mmol, 1.00 eq) in MeOH (15 mL) were added HOAc (1.05 g, 17.5 mmol, 999 μL, 2.00 eq) and tert-butyl 4-formylpiperidine-1-carboxylate (2.42 g, 11.4 mmol, 1.30 eq) at 25 °C, the mixture was stirred at 25 °C for 0.5 h, then NaBH₃CN (2.75 g, 43.7 mmol, 5.00 eq) was added to the mixture at 25 °C. The mixture was stirred at 40 °C for 12 h. LCMS showed that 3-(1-oxo-5-(piperidin-4-yloxy)isoindolin-2-yl)piperidine-2,6-dione (083-4) was consumed completely and the desired mass was detected. Water (100 mL) was added to the solution, the mixture was extracted with ethyl acetate (100 mL*3), the combined organic layers were dried over Na₂SO₄, and concentrated under reduced pressure. The residue was purified by column chromatography (SiO₂, Dichloromethane/Methanol=10/1 to 6/1) to give the title compound (083-5) (3.30 g, 5.37 mmol, 61.4% yield, 87.9% purity) as a white solid. LCMS (ES+): m/z 541.3 [M+H]+.

Synthesis of 3-(1-oxo-5-((1-(piperidin-4-ylmethyl)piperidin-4-yl)oxy)isoindolin-2-yl)piperidine-2,6-dione (083-6). To a solution of tert-butyl 4-((4-((2-(2,6-dioxopiperidin-3-yl)-1-oxoisoindolin-5-yl)oxy)piperidin-1-yl)methyl)piperidine-1-carboxylate (083-5) (2.80 g, 5.18 mmol, 1.00 eq) in DCM (15 mL) was added TFA (2.95 g, 25.9 mmol, 1.92 mL, 5.00 eq) at 25 °C. The mixture was stirred at 25 °C for 2 h. TLC (SiO₂, Dichloromethane/Petroleum ether=4/1, Rf=0.24) showed that tert-butyl 4-((4-((2-(2,6-dioxopiperidin-3-yl)-1-oxoisoindolin-5-yl)oxy)piperidin-1-yl)methyl)piperidine-1-carboxylate (083-5) was consumed completely and one new spot formed. The reaction was concentrated under reduced pressure to give the title compound (083-6) (6.20 g, crude) as a white solid. LCMS (ES+): m/z 441.1 [M+H]+.

Synthesis of 3-(5-((1-((1-(2-(4-((4-([1,1′-biphenyl]-3-yl)-5-chloropyrimidin-2-yl)amino)piperidin-1-yl)-2-oxoethyl)piperidin-4-yl)methyl)piperidin-4-yl)oxy)-1-oxoisoindolin-2-yl)piperidine-2,6-dione (Compound 85). To a solution of 3-(1-oxo-5-((1-(piperidin-4-ylmethyl)piperidin-4-yl)oxy)isoindolin-2-yl)piperidine-2,6-dione (083-6) (200 mg, 453 μmol, 1.00 eq) in DMF (5.00 mL) was added DIEA (58.6 mg, 453 μmol, 79.0 μL, 1.00 eq) and 2-bromo-1-[4-[[5-chloro-4-(3-phenylphenyl)pyrimidin-2-yl]amino]-1-piperidyl]ethanone (220 mg, 453 μmol, 1.00 eq) (prepared as described in Example 83) at 25 °C. The mixture was stirred at 40 °C for 12 h. LCMS showed that 3-(1-oxo-5-((1-(piperidin-4-ylmethyl)piperidin-4-yl)oxy)isoindolin-2-yl)piperidine-2,6-dione (083-6) was consumed completely and the desired mass was detected. Water (50 mL) was added to the solution, the mixture was extracted with ethyl acetate (50 mL*3), the combined organic layers were dried over Na₂SO₄, and concentrated under reduced pressure. The residue was purified by prep-HPLC (column: Phenomenex Luna C18 75*30 mm*3 μm; mobile phase: [water(FA)-ACN]; B %: 25%-45%, 8 min) to give the title compound (Compound 85) (37.6 mg, 42.1 μmol, 9.28% yield, 99.9% purity, 1.00 FA) as a white solid. LCMS (ES+): m/z 845.4 [M+H]+.

### Example 84. Synthesis of 3-(5-(1′-(3-(4-((4-([1,1′-biphenyl]-3-yl)-5-chloropyrimidin-2-yl)amino)piperidin-1-yl)-3-oxopropyl)-[1,4′-bipiperidin]-4-yl)-1-oxoisoindolin-2-yl)piperidine-2,6-dione (Compound 86)

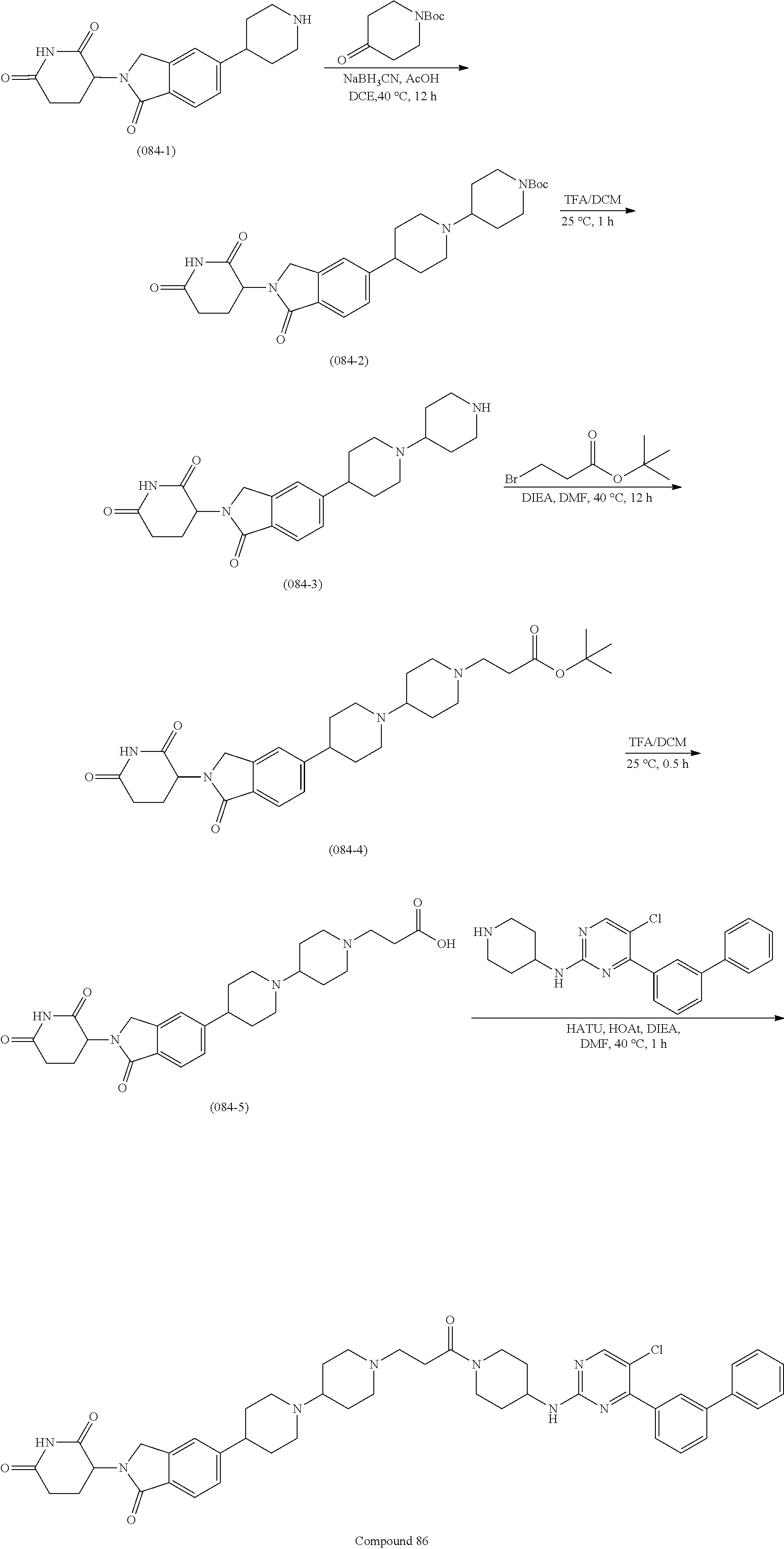

Synthesis of tert-butyl 4-(2-(2,6-dioxopiperidin-3-yl)-1-oxoisoindolin-5-yl)-[1,4′-bipiperidine]-1′-carboxylate (084-2). To a solution of 3-(1-oxo-5-(piperidin-4-yl)isoindolin-2-yl)piperidine-2,6-dione (084-1) (1.50 g, 4.58 mmol, 1.00 eq) in DCE (20 mL) were added AcOH (1.38 g, 22.9 mmol, 1.31 mL, 5.00 eq), tert-butyl 4-oxopiperidine-1-carboxylate (6.39 g, 32.0 mmol, 7.00 eq) and NaBH₃CN (1.44 g, 22.9 mmol, 5.00 eq) at 25 °C. The mixture was stirred at 40 °C for 12 h. LCMS showed that the desired compound was detected. The reaction mixture was diluted with H₂O 40 mL and extracted with EtOAc (50 mL*3). The combined organic layers were washed with brine 50 mL, dried over Na₂SO₄, filtered and the filtrate was concentrated under reduced pressure. The residue was purified by column chromatography (SiO₂, Dichloromethane/Methanol=10/1 to 5/1) to give the title compound (084-2) (700 mg, 1.23 mmol, 26.7% yield, 89.5% purity) as a white solid. LCMS (ES+): m/z 511.3 [M+H]+.

Synthesis of 3-(5-([1,4′-bipiperidin]-4-yl)-1-oxo-2,3-dihydro-1H-inden-2-yl)piperidine-2,6-dione (084-3). To a solution of tert-butyl 4-(2-(2,6-dioxopiperidin-3-yl)-1-oxoisoindolin-5-yl)-[1,4′-bipiperidine]-1′-carboxylate (084-2) (0.500 g, 979 μmol, 1.00 eq) in DCM (5 mL) was added TFA (1.93 g, 16.8 mmol, 1.25 mL, 17.2 eq) at 25 °C. The mixture was stirred at 25 °C for 1 h. LCMS showed that the desired compound was detected. The reaction concentrated under reduced pressure to give the title compound (084-3) (400 mg, crude) as a yellow oil. LCMS (ES+): m/z 411.2 [M+H]+.

Synthesis of tert-butyl 3-(4-(2-(2,6-dioxopiperidin-3-yl)-1-oxoisoindolin-5-yl)-[1,4′-bipiperidin]-1′-yl)propanoate (084-4). To a solution of 3-(5-([1,4′-bipiperidin]-4-yl)-1-oxo-2,3-dihydro-1H-inden-2-yl)piperidine-2,6-dione (084-3) (300 mg, 730 μmol, 1.00 eq) in DMF (5 mL) was added DIEA (472 mg, 3.65 mmol, 636 μL, 5.00 eq) and tert-butyl 3-bromopropanoate (305 mg, 1.46 mmol, 244 μL, 2.00 eq) at 25 °C. The mixture was stirred at 40 °C for 12 h. LCMS showed the desired mass was detected. The reaction mixture was diluted with H₂O 40 mL and extracted with EtOAc (30 mL*3). The combined organic layers were washed with aqueous NaCl 30 mL, dried over Na₂SO₄, filtered and concentrated under reduced pressure. The residue was purified by prep-TLC (SiO₂, Dichloromethane/Methanol=10/1 to 5/1) to give the title compound (084-4) (220 mg, 199 μmol, 27.2% yield, 48.7% purity) as a yellow solid. LCMS (ES+): m/z 539.3 [M+H]+.

Synthesis of 3-(4-(2-(2,6-dioxopiperidin-3-yl)-1-oxoisoindolin-5-yl)-[1,4′-bipiperidin]-1′-yl)propanoic acid (084-5). To a solution of tert-butyl 3-(4-(2-(2,6-dioxopiperidin-3-yl)-1-oxoisoindolin-5-yl)-[1,4′-bipiperidin]-1′-yl)propanoate (084-4) (220 mg, 408 μmol, 1.00 eq) in DCM (5 mL) was added TFA (46.5 mg, 408 μmol, 30.2 μL, 1.00 eq) at 25 °C. The mixture was stirred at 25 °C for 0.5 h. LCMS showed that the desired mass was detected. The reaction concentrated under reduced pressure to give the title compound (084-5) (200 mg, crude) as a brown oil. LCMS (ES+): m/z 483.4 [M+H]+.

Synthesis of 3-(5-(1′-(3-(4-((4-([1,1′-biphenyl]-3-yl)-5-chloropyrimidin-2-yl)amino)piperidin-1-yl)-3-oxopropyl)-[1,4′-bipiperidin]-4-yl)-1-oxoisoindolin-2-yl)piperidine-2,6-dione (Compound 86). To a solution of 3-(4-(2-(2,6-dioxopiperidin-3-yl)-1-oxoisoindolin-5-yl)-[1,4′-bipiperidin]-1′-yl)propanoic acid (084-5) (110 mg, 227 μmol, 1.00 eq) in DMF (1 mL) was added DIEA (88.3 mg, 683 μmol, 119 μL, 3.00 eq) to adjust pH to 7-8. Then 5-chloro-4-(3-phenylphenyl)-N-(4-piperidyl)pyrimidin-2-amine (Compound B) (83.1 mg, 227 μmol, 1.00 eq), DIEA (88.3 mg, 683 μmol, 119 μL, 3.00 eq), HATU (130 mg, 341 μmol, 1.50 eq) and HOAt (46.5 mg, 341 μmol, 47.8 μL, 1.50 eq) were added to the above mixture at 25 °C. The mixture was stirred at 40 °C for 1 h. LCMS showed the desired mass was detected. The residue was purified by prep-HPLC (column: Phenomenex Luna 80*30 mm*3 μm; mobile phase: [water (FA)-ACN]; B %: 20%-50%, 8 min) to give the title compound (Compound 86) (3.50 mg, 4.06 μmol, 2.04% yield, 96.2% purity) as a white solid. LCMS (ES+): m/z 829.4 [M+H]+.

### Example 85. Synthesis of 3-(5-((1′-(2-(4-((4-([1,1′-biphenyl]-3-yl)-5-chloropyrimidin-2-yl)amino)piperidin-1-yl)-2-oxoethyl)-[1,4′-bipiperidin]-4-yl)oxy)-1-oxoisoindolin-2-yl)piperidine-2,6-dione (Compound 87)

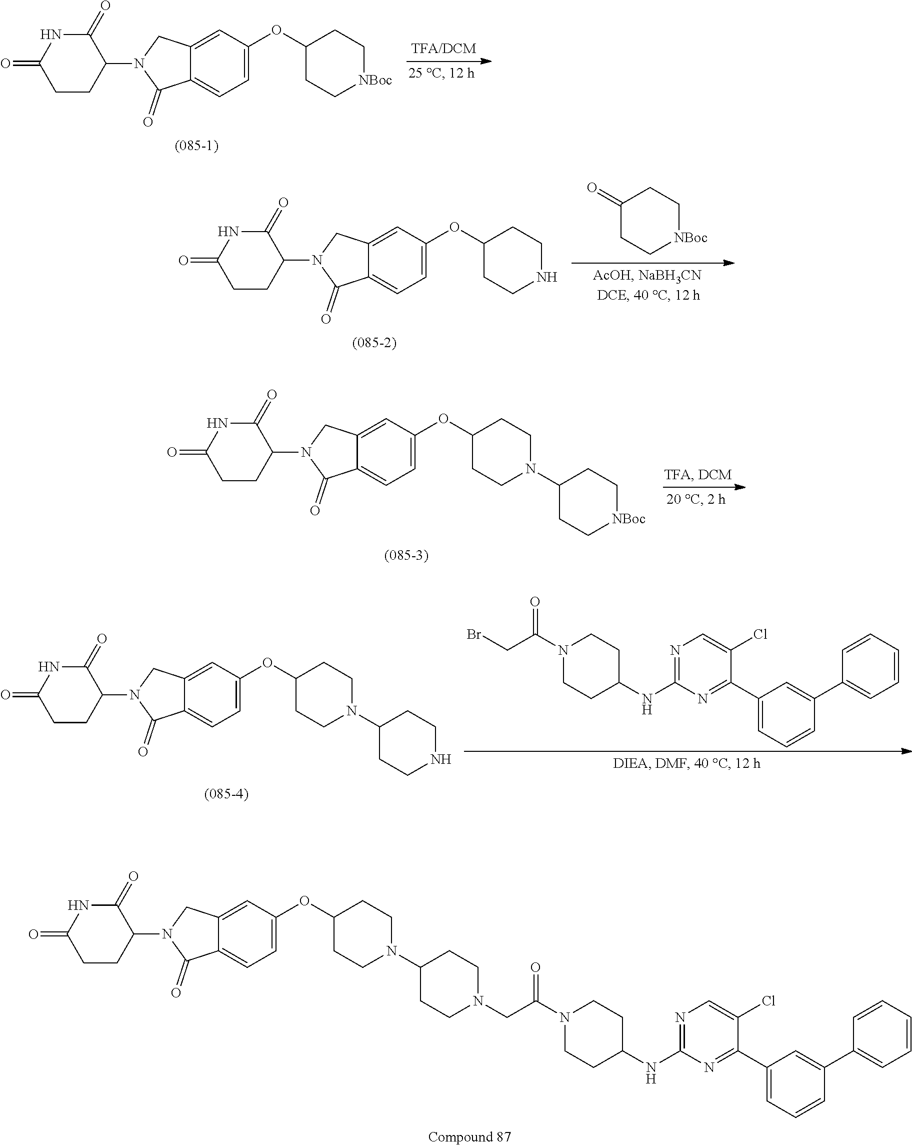

Synthesis of 3-(1-oxo-5-(piperidin-4-yloxy)isoindolin-2-yl)piperidine-2,6-dione (085-2). To a solution of tert-butyl 4-((2-(2,6-dioxopiperidin-3-yl)-1-oxoisoindolin-5-yl)oxy)piperidine-1-carboxylate (085-1) (8.00 g, 18.0 mmol, 1.00 eq) in DCM (50.0 mL) was added TFA (23.1 g, 202 mmol, 15.0 mL, 11.2 eq) at 25 °C. The mixture was stirred at 25 °C for 12 h. LCMS showed that tert-butyl 4-((2-(2,6-dioxopiperidin-3-yl)-1-oxoisoindolin-5-yl)oxy)piperidine-1-carboxylate (085-1) was consumed completely and the desired mass was detected. The reaction mixture was concentrated under reduced pressure to give the title compound (085-2) (12.5 g, crude) as a yellow oil. LCMS (ES+): m/z 344.1 [M+H]+.

Synthesis of tert-butyl 4-((2-(2,6-dioxopiperidin-3-yl)-1-oxoisoindolin-5-yl)oxy)-[1,4′-bipiperidine]-1′-carboxylate (085-3). To a solution of 3-(1-oxo-5-(piperidin-4-yloxy)isoindolin-2-yl)piperidine-2,6-dione (085-2) (8.00 g, 23.30 mmol, 1.00 eq) in DCE (20 mL) were added HOAc (4.20 g, 69.9 mmol, 4.00 mL, 3.00 eq) and tert-butyl 4-oxopiperidine-1-carboxylate (32.5 g, 163.1 mmol, 7.00 eq) at 25 °C, the mixture was stirred at 25 °C for 0.5 h, then NaBH₃CN (7.32 g, 116 mmol, 5.00 eq) was added to the mixture at 25 °C. The mixture was stirred at 40 °C for 11.5 h. LCMS showed that 3-(1-oxo-5-(piperidin-4-yloxy)isoindolin-2-yl)piperidine-2,6-dione (085-2) was consumed completely and the desired mass was detected. Water (100 mL) was added to the solution, the mixture was extracted with ethyl acetate (100 mL*3), the combined organic layers were dried over Na₂SO₄, and concentrated under reduced pressure. The residue was purified by column chromatography (SiO₂, Dichloromethane/Methanol=10/1 to 5/1) to give the title compound (085-3) (7.00 g, 12.9 mmol, 55.7% yield, 97.6% purity) as a white solid. LCMS (ES+): m/z 527.3 [M+H]+.

Synthesis of 3-(5-([1,4′-bipiperidin]-4-yloxy)-1-oxoisoindolin-2-yl)piperidine-2,6-dione (085-4). To a solution of tert-butyl 4-((2-(2,6-dioxopiperidin-3-yl)-1-oxoisoindolin-5-yl)oxy)-[1,4′-bipiperidine]-1′-carboxylate (085-3) (7.00 g, 13.3 mmol, 1.00 eq) in DCM (15 mL) was added TFA (7.58 g, 66.5 mmol, 4.94 mL, 5.00 eq) at 20 °C. The mixture was stirred at 20 °C for 2 h. LCMS showed that tert-butyl 4-((2-(2,6-dioxopiperidin-3-yl)-1-oxoisoindolin-5-yl)oxy)-[1,4′-bipiperidine]-1′-carboxylate (085-3) was consumed completely and desired mass was detected. The reaction concentrated under reduced pressure to give the title compound (085-4) (14.0 g, crude) as a white solid. LCMS (ES+): m/z 427.2 [M+H]+.

Synthesis of 3-(5-((1′-(2-(4-((4-([1,1′-biphenyl]-3-yl)-5-chloropyrimidin-2-yl)amino)piperidin-1-yl)-2-oxoethyl)-[1,4′-bipiperidin]-4-yl)oxy)-1-oxoisoindolin-2-yl)piperidine-2,6-dione (Compound 87). To a solution of 3-(5-([1,4′-bipiperidin]-4-yloxy)-1-oxoisoindolin-2-yl)piperidine-2,6-dione (085-4) (200 mg, 468.9 μmol, 1.00 eq) in DMF (5 mL) was added DIEA (121 mg, 937 μmol, 163 μL, 2.00 eq) and 2-bromo-1-[4-[[5-chloro-4-(3-phenylphenyl)pyrimidin-2-yl]amino]-1-piperidyl]ethanone (273 mg, 562 μmol, 1.20 eq) (prepared as described in Example 83) at 25 °C. The mixture was stirred at 40 °C for 12 h. LCMS showed that 3-(5-([1,4′-bipiperidin]-4-yloxy)-1-oxoisoindolin-2-yl)piperidine-2,6-dione (085-4) was consumed completely and the desired mass was detected. The reaction concentrated under reduced pressure. The residue was purified by prep-HPLC (column: Phenomenex Luna C18 75*30 mm*3 μm; mobile phase: [water(FA)-ACN]; B %: 15%-55%, 8 min) to give the title compound (Compound 87) (96.2 mg, 114.22 μmol, 24.36% yield, 98.71% purity) as a white solid. LCMS (ES+): m/z 831.3 [M+H]+.

### Example 86. Synthesis of 3-(5-(1-(2-(4-(4-((4-([1,1′-biphenyl]-3-yl)-5-chloropyrimidin-2-yl)amino)piperidine-1-carbonyl)piperidin-1-yl)ethyl)piperidin-4-yl)-1-oxoisoindolin-2-yl)piperidine-2,6-dione (Compound 88)

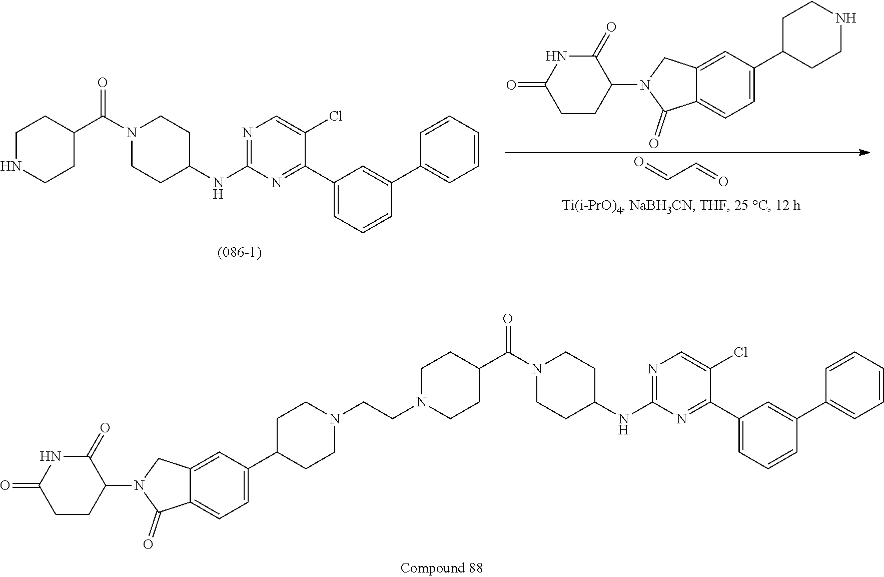

Synthesis of 3-(5-(1-(2-(4-(4-((4-([1,1′-biphenyl]-3-yl)-5-chloropyrimidin-2-yl)amino)piperidine-1-carbonyl)piperidin-1-yl)ethyl)piperidin-4-yl)-1-oxoisoindolin-2-yl)piperidine-2,6-dione (Compound 88). To a solution of (4-((4-([1,1′-biphenyl]-3-yl)-5-chloropyrimidin-2-yl)amino)piperidin-1-yl)(piperidin-4-yl)methanone (Compound 2) (086-1) (150 mg, 315 μmol, 1.00 eq) and 3-[1-oxo-5-(4-piperidyl)isoindolin-2-yl]piperidine-2,6-dione (103 mg, 315 μmol, 1.00 eq) in THF (5.00 mL) were added oxaldehyde (18.2 mg, 315 μmol, 16.4 μL, 1.00 eq) Ti(i-PrO)₄ (447 mg, 1.58 mmol, 465 μL, 5.00 eq) at 25 °C, the mixture was stirred at 25 °C for 0.5 h, then NaBH₃CN (99.0 mg, 1.58 mmol, 5.00 eq) was added to the mixture at 25 °C. The mixture was stirred at 40 °C for 11.5 h. LCMS showed that (4-((4-([1,1′-biphenyl]-3-yl)-5-chloropyrimidin-2-yl)amino)piperidin-1-yl)(piperidin-4-yl)methanone (Compound 2) (086-1) was consumed completely and desired mass was detected. The reaction mixture was diluted with H₂O 10.0 mL, and extracted with EtOAc (20.0 mL*3). The combined organic layers were dried over Na₂SO₄, filtered and concentrated under reduced pressure. The residue was purified by prep-HPLC (column: Phenomenex Luna C18 200*40 mm*10 μm; mobile phase: [water (FA)-ACN]; B %: 30%-60%, 8 min) to give the title compound (Compound 88) (27.0 mg, 30.6 μmol, 9.73% yield, 99.4% purity, 1.00 FA) as an orange solid. LCMS (ES+): m/z 829.4 [M+H]+

### Example 87. Synthesis of 3-(5-((1-(2-(4-(4-((4-([1,1′-biphenyl]-3-yl)-5-chloropyrimidin-2-yl)amino)piperidine-1-carbonyl)piperidin-1-yl)ethyl)piperidin-4-yl)oxy)-1-oxoisoindolin-2-yl)piperidine-2,6-dione (Compound 89)

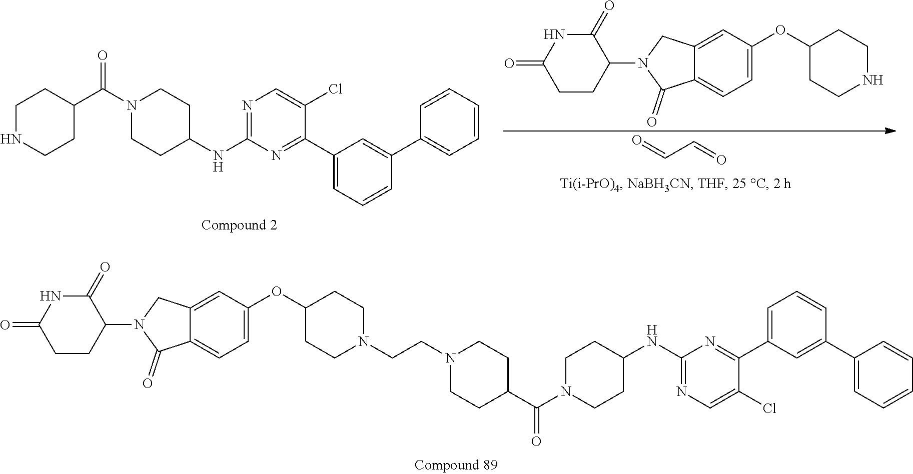

Synthesis of 3-(5-((1-(2-(4-(4-((4-([1,1′-biphenyl]-3-yl)-5-chloropyrimidin-2-yl)amino)piperidine-1-carbonyl)piperidin-1-yl)ethyl)piperidin-4-yl)oxy)-1-oxoisoindolin-2-yl)piperidine-2,6-dione (Compound 89). To a solution of (4-((4-([1,1′-biphenyl]-3-yl)-5-chloropyrimidin-2-yl)amino)piperidin-1-yl)(piperidin-4-yl)methanone (Compound 2) (200 mg, 420 μmol, 1.00 eq), 3-[1-oxo-5-(4-piperidyloxy)isoindolin-2-yl]piperidine-2,6-dione (144 mg, 420 μmol, 1.00 eq) and oxaldehyde (24.3 mg, 420 μmol, 21.9 μL, 1.00 eq) in THF (5.00 mL) was added Ti(i-PrO)₄ (597 mg, 2.10 mmol, 620 μL, 5.00 eq) at 25 °C, then the mixture was stirred at 25 for 0.5 h, then NaBH₃CN (132 mg, 2.10 mmol, 5.00 eq) was added to the mixture at 25 °C. The mixture was stirred at 25 °C for 1.5 h. LCMS showed (4-((4-([1,1′-biphenyl]-3-yl)-5-chloropyrimidin-2-yl)amino)piperidin-1-yl)(piperidin-4-yl)methanone (Compound 2) was consumed completely and the desired mass was detected. The reaction mixture was diluted with H₂O 10.0 mL, and extracted with EtOAc (20.0 mL*3). The combined organic layers were dried over Na₂SO₄, filtered and the filtrate was concentrated under reduced pressure. The residue was purified by prep-HPLC (column: Phenomenex Luna C18 200*40 mm*10 μm; mobile phase: [water(FA)-ACN]; B %: 15%-50%, 8 min) to give the title compound (Compound 89) (25.3 mg, 27.4 μmol, 6.53% yield, 93.7% purity, 0.400 FA) as a white solid. LCMS (ES+): m/z 845.4 [M+H]+.

### Example 88. Synthesis of 3-(5-((1′-(3-(4-((4-([1,1′-biphenyl]-3-yl)-5-chloropyrimidin-2-yl)amino)piperidin-1-yl)-3-oxopropyl)-[1,4′-bipiperidin]-4-yl)oxy)-1-oxoisoindolin-2-yl)piperidine-2,6-dione (Compound 90)

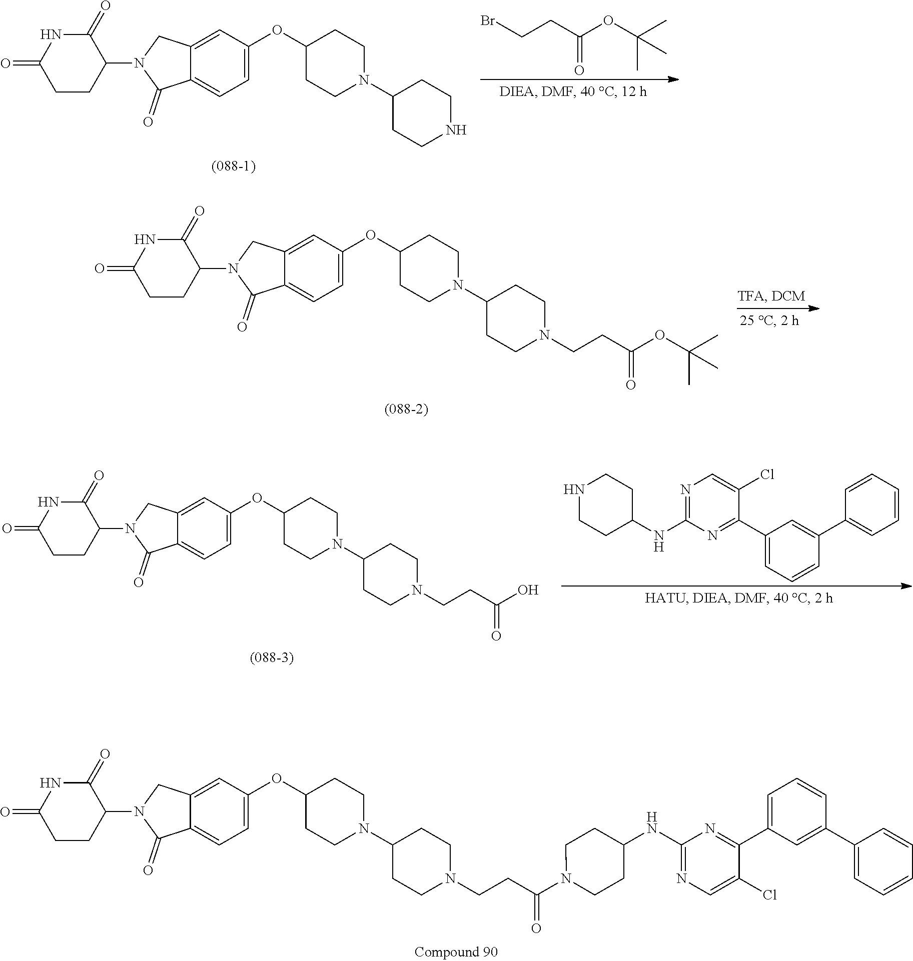

Synthesis of tert-butyl 3-(4-((2-(2,6-dioxopiperidin-3-yl)-1-oxoisoindolin-5-yl)oxy)-[1,4′-bipiperidin]-1′-yl)propanoate (088-2). To a solution of 3-(5-([1,4′-bipiperidin]-4-yloxy)-1-oxoisoindolin-2-yl)piperidine-2,6-dione (088-1) (500 mg, 1.17 mmol, 1.00 eq) (prepared as described in Example 85) in DMF (5.00 mL) were added DIEA (757 mg, 5.86 mmol, 1.02 mL, 5.00 eq) and tert-butyl 3-bromopropanoate (367 mg, 1.76 mmol, 293 μL, 1.50 eq) at 25 °C. The mixture was stirred at 40 °C for 12 h. LCMS showed 3-(5-([1,4′-bipiperidin]-4-yloxy)-1-oxoisoindolin-2-yl)piperidine-2,6-dione (088-1) was consumed completely and the desired mass was detected. The reaction mixture was diluted with ice water 25.0 mL, and filtered to get crude product. The crude product was purified by re-crystallization from water (50.0 mL) at 25 °C to give the title compound (088-2) (500 mg, 644 μmol, 10.9% yield, 71.4% purity) as a white solid. LCMS (ES+): m/z 555.3 [M+H]+.

Synthesis of 3-(4-((2-(2,6-dioxopiperidin-3-yl)-1-oxoisoindolin-5-yl)oxy)-[1,4′-bipiperidin]-1′-yl)propanoic acid (088-3). To a solution of tert-butyl 3-(4-((2-(2,6-dioxopiperidin-3-yl)-1-oxoisoindolin-5-yl)oxy)-[1,4′-bipiperidin]-1′-yl)propanoate (088-2) (450 mg, 811 μmol, 1.00 eq) in DCM (6.00 mL) was added TFA (3.07 g, 26.9 mmol, 2.00 mL, 33.1 eq) at 25 °C. The mixture was stirred at 25 °C for 2 h. LCMS showed tert-butyl 3-(4-((2-(2,6-dioxopiperidin-3-yl)-1-oxoisoindolin-5-yl)oxy)-[1,4′-bipiperidin]-1′-yl)propanoate (088-2) was consumed completely and the desired mass was detected. The reaction was concentrated under reduced pressure to give the title compound (088-3) (500 mg, crude) as a yellow oil. LCMS (ES+): m/z 499.2 [M+H]+.

Synthesis of 3-(5-((1′-(3-(4-((4-([1,1′-biphenyl]-3-yl)-5-chloropyrimidin-2-yl)amino)piperidin-1-yl)-3-oxopropyl)-[1,4′-bipiperidin]-4-yl)oxy)-1-oxoisoindolin-2-yl)piperidine-2,6-dione (Compound 90). To a solution of 3-(4-((2-(2,6-dioxopiperidin-3-yl)-1-oxoisoindolin-5-yl)oxy)-[1,4′-bipiperidin]-1′-yl)propanoic acid (088-3) (130 mg, 260 μmol, 1.00 eq) and 4-([1,1′-biphenyl]-3-yl)-5-chloro-N-(piperidin-4-yl)pyrimidin-2-amine (Compound B) (95.1 mg, 260 μmol, 1.00 eq) in DMF (3.00 mL) was added DIEA (101 mg, 782 μmol, 136 μL, 3.00 eq) and HATU (148 mg, 391 μmol, 1.50 eq) at 25 °C. The mixture was stirred at 40 °C for 2 h. LCMS showed that 3-(4-((2-(2,6-dioxopiperidin-3-yl)-1-oxoisoindolin-5-yl)oxy)-[1,4′-bipiperidin]-1′-yl)propanoic acid (088-3) was consumed completely and the desired mass was detected. The reaction mixture was diluted with H₂O 10.0 mL, and extracted with EtOAc (20.0 mL*3). The combined organic layers were dried over Na₂SO₄, filtered and concentrated under reduced pressure. The residue was purified by prep-HPLC. (column: Phenomenex luna C18 80*40 mm*3 μm; mobile phase: [water(FA)-ACN]; gradient: 10%-50% B over 8 min) to give the title compound (Compound 90) (40.3 mg, 44.9 μmol, 17.2% yield, 98.9% purity, 0.900 FA) as a light yellow solid. LCMS (ES+): m/z 845.4 [M+H]+.

### Example 89. Synthesis of 1-(4-((4-([1,1′-biphenyl]-3-yl)-5-chloropyrimidin-2-yl)amino)piperidin-1-yl)-3-(1-methylpiperidin-4-yl)propan-1-one (Compound 10)

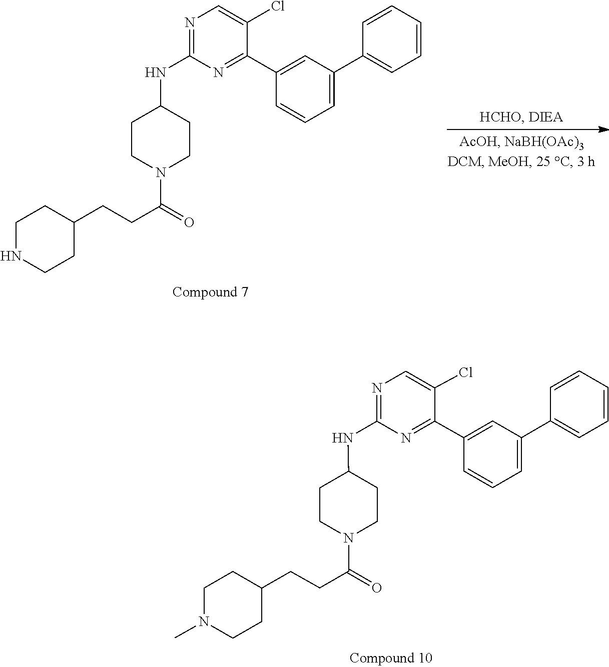 [SMILES: O=C(CCC1CCNCC1)N1CCC(Nc2ncc(Cl)c(-c3cccc(-c4ccccc4)c3)n2)CC1 ; CN1CCC(CCC(=O)N2CCC(Nc3ncc(Cl)c(-c4cccc(-c5ccccc5)c4)n3)CC2)CC1]

Synthesis of 1-(4-((4-([1,1′-biphenyl]-3-yl)-5-chloropyrimidin-2-yl)amino)piperidin-1-yl)-3-(1-methylpiperidin-4-yl)propan-1-one (Compound 10). To a solution of 1-(4-((4-([1,1′-biphenyl]-3-yl)-5-chloropyrimidin-2-yl)amino)piperidin-1-yl)-3-(piperidin-4-yl)propan-1-one (Compound 7) (230 mg, 372 μmol, 1.00 eq, TFA) in DCM (2 mL) and MeOH (1 mL) were added aq. HCHO (513 mg, 6.33 mmol, 4.47 μL, 37.0% purity, 17.0 eq) and DIEA (48.1 mg, 372 μmol, 64.8 μL, 1.00 eq) at 25 °C. The mixture was stirred at 25 °C for 0.5 h. AcOH (22.4 mg, 372 μmol, 21.3 μL, 1.00 eq) was added to adjust pH to 5 at 25 °C. The mixture was stirred at 25 °C for 0.5 h. NaBH(OAc)₃ (94.6 mg, 447 μmol, 1.20 eq) was added to the mixture at 25 °C. The mixture was stirred at 25 °C for 2 h. LCMS showed 1-(4-((4-([1,1′-biphenyl]-3-yl)-5-chloropyrimidin-2-yl)amino)piperidin-1-yl)-3-(piperidin-4-yl)propan-1-one (Compound 7) was consumed completely and the desired mass was detected. The reaction mixture was concentrated under reduced pressure. The residue was purified by prep-HPLC (column: Phenomenex Luna C18 75*30 mm*3 um; mobile phase: [water (FA)-ACN]; B %: 35%-75%, 8 min) to give the title compound (Compound 10) (73.4 mg, 140 mol, 37.7% yield, 98.9% purity) as a brown oil. LCMS (ES+): m/z 518.2 [M+H]+.

### Example 90. cis-3-((5-fluoro-4-(1-(4-fluorophenyl)piperidin-3-yl)pyrimidin-2-yl)amino)cyclohexane-1-carboxamide (Compound 91)

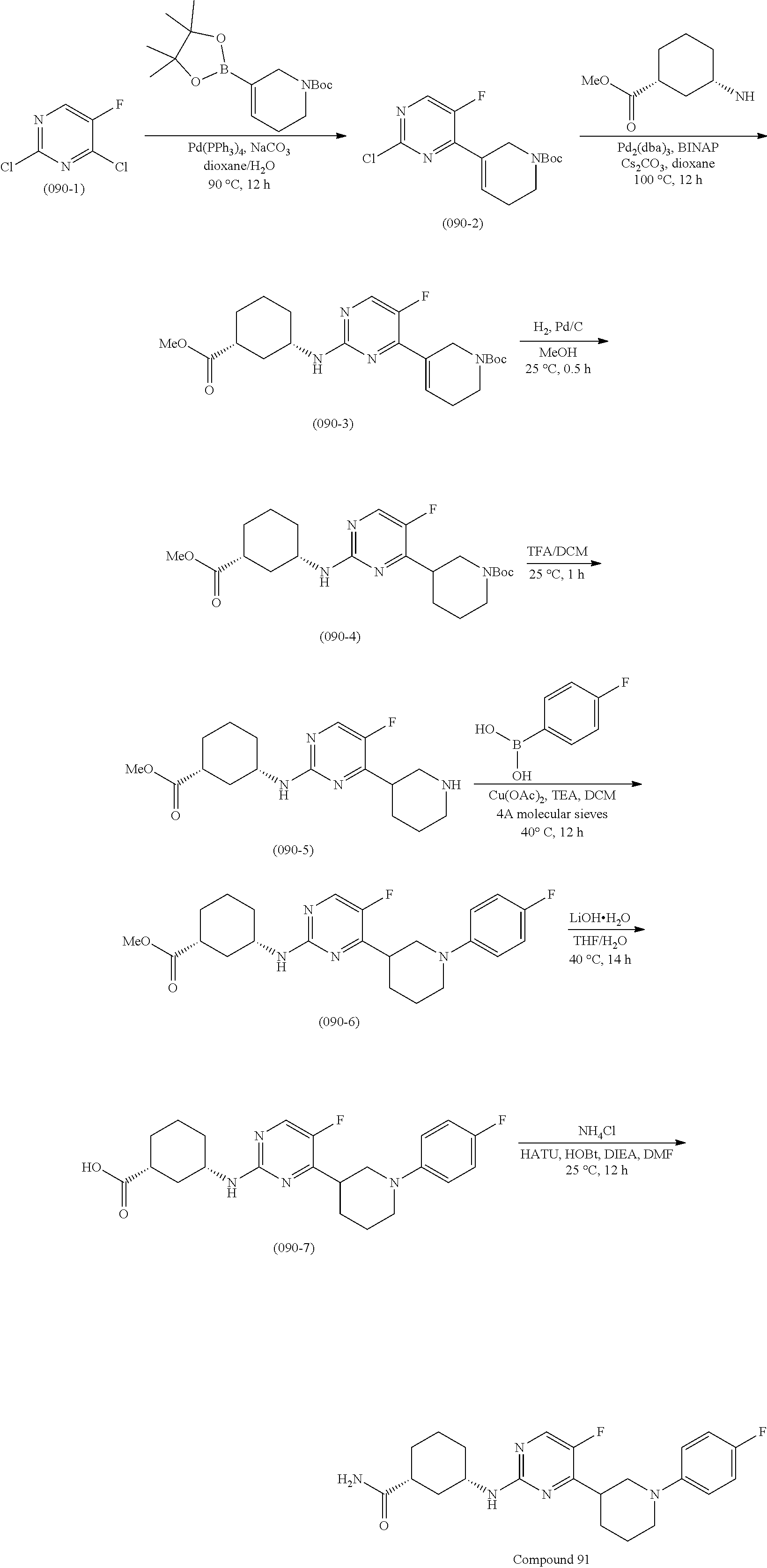

Synthesis of tert-butyl 5-(2-chloro-5-fluoropyrimidin-4-yl)-3,6-dihydropyridine-1 (2H)-carboxylate (090-2). To a solution of tert-butyl 5-(4,4,5,5-tetramethyl-1,3,2-dioxaborolan-2-yl)-3,6-dihydropyridine-1 (2H)-carboxylate (3.00 g, 9.70 mmol, 1.00 eq) in dioxane (20 mL) and H₂O (4 mL) were added 2,4-dichloro-5-fluoro-pyrimidine (090-1) (1.62 g, 9.70 mmol, 1.00 eq), Na₂CO₃ (2.06 g, 19.4 mmol, 2.00 eq) and Pd(PPh₃)₄ (224 mg, 194 mol, 0.02 eq) at 25 °C. The mixture was stirred at 90 °C for 12 h. LCMS showed that the desired mass was detected. Water (15 mL) was added to the mixture at 25 °C. Then the reaction mixture was extracted with EtOAc (10 mL*3). The organic layers were dried over anhydrous sodium sulfate, filtered and the filtrate was concentrated under reduced pressure to give a residue. The residue was purified by column chromatography (SiO₂, Petroleum ether/Ethyl acetate=5/1 to 3/1) to give the title compound (090-2) (2.30 g, 6.68 mmol, 68.8% yield, 91.1% purity) as a yellow oil. LCMS (ES+): m/z 258.0 [M+H−56]+.

Synthesis of cis-tert-butyl 5-(5-fluoro-2-((3-(methoxycarbonyl)cyclohexyl)amino)pyrimidin-4-yl)-3,6-dihydropyridine-1 (2H)-carboxylate (090-3). To a solution of tert-butyl 5-(2-chloro-5-fluoropyrimidin-4-yl)-3,6-dihydropyridine-1 (2H)-carboxylate (090-2) (1.70 g, 5.42 mmol, 1.20 eq) in dioxane (20 mL) were added cis-methyl 3-aminocyclohexanecarboxylate (710 mg, 4.52 mmol, 1.00 eq), Cs₂CO₃ (4.41 g, 13.6 mmol, 3.00 eq), BINAP (281 mg, 452 μmol, 0.10 eq) and Pd₂(dba)₃ (413 mg, 452 μmol, 0.10 eq) at 25 °C. The mixture was stirred at 100 °C for 12 h. LCMS showed that the desired mass was detected. Water (15 mL) was added to the mixture at 25 °C. Then the reaction mixture was extracted with EtOAc (10 mL*3). The organic layers were dried over anhydrous sodium sulfate, filtered and the filtrate was concentrated under reduced pressure to give a residue. The residue was purified by column chromatography (SiO₂, Petroleum ether/Ethyl acetate=5/1 to 3/1) to give the title compound (090-3) (600 mg, 1.21 mmol, 26.9% yield, 87.8% purity) as a yellow oil. LCMS (ES+): m/z 435.1 [M+H]+.

Synthesis of cis-tert-butyl 3-(5-fluoro-2-((3-(methoxycarbonyl)cyclohexyl)amino)pyrimidin-4-yl)piperidine-1-carboxylate (090-4). To a solution of cis-tert-butyl 5-(5-fluoro-2-((3-(methoxycarbonyl)cyclohexyl)amino)pyrimidin-4-yl)-3,6-dihydropyridine-1 (2H)-carboxylate (090-3) (600 mg, 1.38 mmol, 1.00 eq) in MeOH (10 mL) were added 10% Pd/C (600 mg, 50% purity) at 25 °C. The mixture was stirred at 25 °C for 15 min under H₂ (15 psi). LCMS showed cis-tert-butyl 5-(5-fluoro-2-((3-(methoxycarbonyl)cyclohexyl)amino)pyrimidin-4-yl)-3,6-dihydropyridine-1 (2H)-carboxylate (090-3) was not consumed completely and the desired mass was not detected. The mixture was added 10% Pd/C (600 mg, 50% purity) at 25 °C. The mixture was stirred at 25 °C for 15 min under H₂ (15 psi). LCMS showed that the desired mass was detected. The reaction mixture was filtered and the filtrate was concentrated under reduced pressure to give the title compound (090-4) (600 mg, 1.01 mmol, 72.9% yield, 73.3% purity) as a yellow oil. LCMS (ES+): m/z 437.2 [M+H]+.

Synthesis of cis-methyl 3-((5-fluoro-4-(piperidin-3-yl)pyrimidin-2-yl)amino)cyclohexane-1-carboxylate (090-5). To a solution of cis-tert-butyl 3-(5-fluoro-2-((3-(methoxycarbonyl)cyclohexyl)amino)pyrimidin-4-yl)piperidine-1-carboxylate (090-4) (600 mg, 1.37 mmol, 1.00 eq) in DCM (6 mL) was added TFA (2 mL) at 25 °C. The mixture was stirred at 25 °C for 1 h. LCMS showed that the desired mass was detected. The reaction mixture was filtered and the filtrate was concentrated under reduced pressure to give a residue to give the title compound (090-5) (600 mg, crude, TFA) as a yellow oil. LCMS (ES+): m/z 337.1 [M+H]+.

Synthesis of cis-methyl 3-((5-fluoro-4-(1-(4-fluorophenyl)piperidin-3-yl)pyrimidin-2-yl)amino)cyclohexane-1-carboxylate (090-6). To a solution of cis-methyl 3-((5-fluoro-4-(piperidin-3-yl)pyrimidin-2-yl)amino)cyclohexane-1-carboxylate (090-5) (600 mg, 1.33 mmol, 1.00 eq, TFA) and (4-fluorophenyl)boronic acid (373 mg, 2.66 mmol, 2.00 eq) in DCM (10 mL) were added TEA (270 mg, 2.66 mmol, 371 μL, 2.00 eq), Cu(OAc)₂ (242 mg, 1.33 mmol, 1.00 eq) and 4 Å MS at 25 °C. The mixture was stirred at 40 °C for 12 h under oxygen (15 psi). LCMS showed that the desired mass was detected. The reaction mixture was concentrated under reduced pressure to remove solvent. Water (9 mL) was added to reaction, the aqueous phase was extracted with EtOAc (8 mL*3), the combined organic layers were dried over Na₂SO₄, filtered and the filtrate was concentrated under reduced pressure. The residue was purified by prep-TLC (SiO₂, Petroleum ether/Ethyl acetate=1/1) to give the title compound (090-6) (320 mg, 662 μmol, 49.7% yield, 89.01% purity) as a white solid. LCMS (ES+): m/z 431.2 [M+H]+.

Synthesis of cis-3-((5-fluoro-4-(1-(4-fluorophenyl)piperidin-3-yl)pyrimidin-2-yl)amino)cyclohexane-1-carboxylic acid (090-7). To a solution of cis-methyl 3-((5-fluoro-4-(1-(4-fluorophenyl)piperidin-3-yl)pyrimidin-2-yl)amino)cyclohexane-1-carboxylate (090-6) (320 mg, 743 μmol, 1.00 eq) in THE (2 mL) and H₂O (1 mL) was added LiOH·H₂O (93.6 mg, 2.23 mmol, 3.00 eq) at 25 °C. The mixture was stirred at 40 °C for 12 h. LCMS showed cis-methyl 3-((5-fluoro-4-(1-(4-fluorophenyl)piperidin-3-yl)pyrimidin-2-yl)amino)cyclohexane-1-carboxylate (090-6) was not consumed completely and the desired mass was detected. The mixture was added LiOH·H₂O (93.6 mg, 2.23 mmol, 3.00 eq) at 25 °C. The mixture was stirred at 40 °C for 2 h. LCMS showed that cis-methyl 3-((5-fluoro-4-(1-(4-fluorophenyl)piperidin-3-yl)pyrimidin-2-yl)amino)cyclohexane-1-carboxylate (090-6) was consumed completely and the desired mass was detected. The reaction mixture was concentrated under reduced pressure to remove solvent. Concentrated HCl 4 ml (10%) was added dropwise until the pH had reached 3. The aqueous solution was extracted with EtOAc (0.5 mL*3). The organic layers were combined, dried over anhydrous Na₂SO₄, filtered and concentrated under reduced pressure to give a residue to give the title compound (090-7) (250 mg, crude) as a yellow solid. LCMS (ES+): m/z 417.1 [M+H]+.

Synthesis of cis-3-((5-fluoro-4-(1-(4-fluorophenyl)piperidin-3-yl)pyrimidin-2-yl)amino)cyclohexane-1-carboxamide (Compound 91). To a solution of cis-3-((5-fluoro-4-(1-(4-fluorophenyl)piperidin-3-yl)pyrimidin-2-yl)amino)cyclohexane-1-carboxylic acid (090-7) (122 mg, 293 μmol, 1.00 eq) and NH₄Cl (62.7 mg, 1.17 mmol, 4.00 eq) in DMF (2 mL) were added DIEA (189 mg, 1.46 mmol, 255 μL, 5.00 eq), HATU (134 mg, 352 μmol, 1.20 eq) and HOBt (19.8 mg, 146 μmol, 0.50 eq) at 25 °C. The mixture was stirred at 25 °C for 12 h. LCMS showed that the desired mass was detected. The reaction mixture was filtered and the filtrate was concentrated under reduced pressure to give a residue. The residue were purified by prep-HPLC (column: C18-6 100*30 mm*5 um; mobile phase: [water(FA)-ACN]; B %: 15%-50%, 8 min) to give to give the title compound (Compound 91) (56.8 mg, 136 μmol, 46.4% yield, 99.4% purity) as a yellow solid. LCMS (ES+): m/z 416.1 [M+H]+.

### Example 91. Synthesis of cis-3-(5-(1-(2-(1-(3-((4-([1,1′-biphenyl]-3-yl)-5-chloropyrimidin-2-yl)amino)cyclohexane-1-carbonyl)piperidin-4-yl)ethyl)piperidin-4-yl)-1-oxoisoindolin-2-yl)piperidine-2,6-dione (Compound 62)

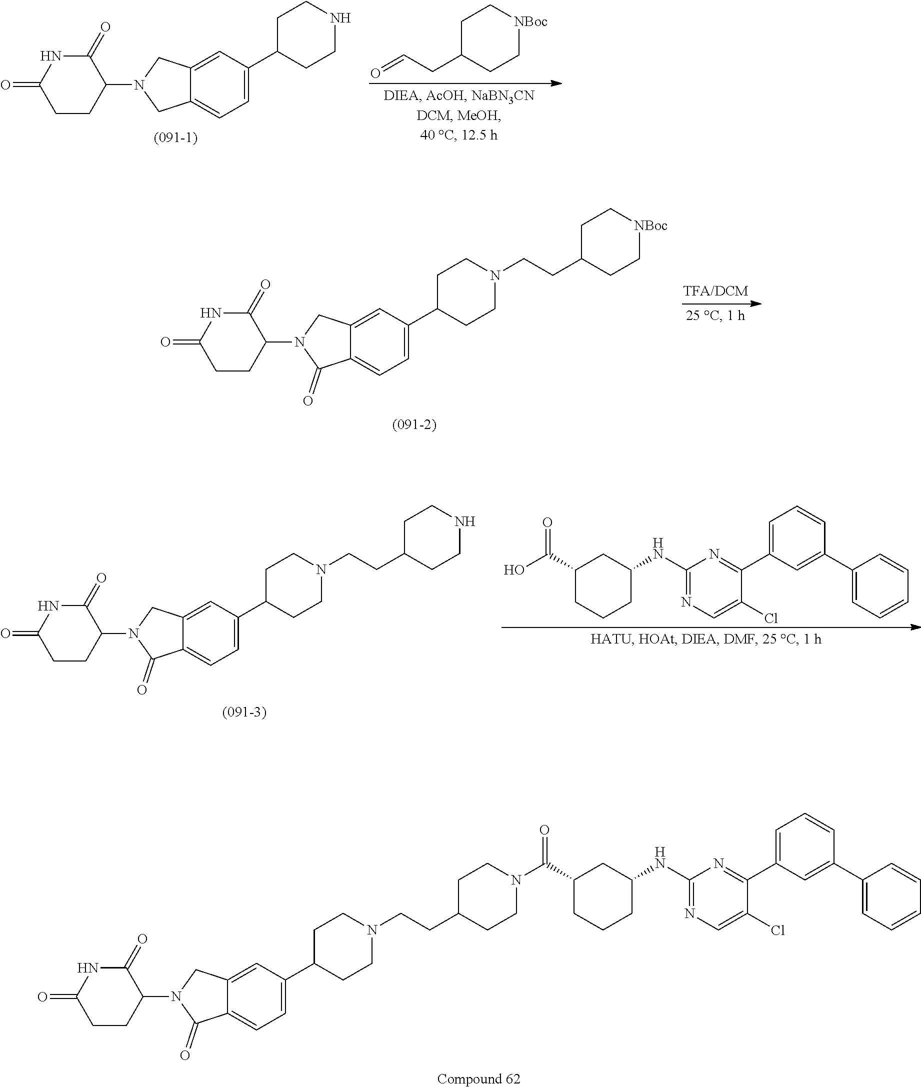

Synthesis of tert-butyl 4-(2-(4-(2-(2,6-dioxopiperidin-3-yl)-1-oxoisoindolin-5-yl)piperidin-1-yl)ethyl)piperidine-1-carboxylate (091-2). To a solution of 3-(1-oxo-5-(piperidin-4-yl)isoindolin-2-yl)piperidine-2,6-dione (091-1) (300 mg, 680 μmol, 1.00 eq, TFA) in MeOH (2 mL) and DCM (1 mL) was added DIEA (263 mg, 2.04 mmol, 355 μL, 3.00 eq) to adjust pH to 8 at 25 °C tert-butyl 4-(2-oxoethyl)piperidine-1-carboxylate (185 mg, 816 μmol, 1.20 eq) was added to the mixture at 25 °C. The mixture was stirred at 25 °C for 0.5 h. AcOH (40.8 mg, 680 μmol, 38.9 μL, 1.00 eq) was added dropwise until pH=5 at 25 °C. The mixture was stirred at 25 °C for 0.5 h. NaBH₃CN (128 mg, 2.04 mmol, 3.00 eq) was added to the mixture at 25 °C. The mixture was stirred at 40 °C for 12 h. LC-MS showed 3-(1-oxo-5-(piperidin-4-yl)isoindolin-2-yl)piperidine-2,6-dione (091-1) consumed completely and the desired mass was detected. The reaction mixture was concentrated under reduced pressure. The residue was purified by prep-TLC (SiO₂, Dichloromethane:Methanol=10:1) to give the title compound (091-2) (360 mg, crude) as a yellow oil. LCMS (ES+): m/z 539.2 [M+H]+.

Synthesis of 3-(1-oxo-5-(1-(2-(piperidin-4-yl)ethyl)piperidin-4-yl)isoindolin-2-yl)piperidine-2,6-dione (091-3). A solution of tert-butyl 4-(2-(4-(2-(2,6-dioxopiperidin-3-yl)-1-oxoisoindolin-5-yl)piperidin-1-yl)ethyl)piperidine-1-carboxylate (091-2) (360 mg, 668 μmol, 1.00 eq) in DCM (5 mL) and TFA (1 mL) was stirred at 25 °C for 1 h. LCMS showed tert-butyl 4-(2-(4-(2-(2,6-dioxopiperidin-3-yl)-1-oxoisoindolin-5-yl)piperidin-1-yl)ethyl)piperidine-1-carboxylate (091-2) was consumed completely and the desired mass was detected. The reaction mixture was concentrated under reduced pressure to give the title compound (091-3) (365 mg, crude, TFA) as a yellow oil. LCMS (ES+): m/z 439.2 [M+H]+

Synthesis of cis-3-(5-(1-(2-(1-(3-((4-([1,1′-biphenyl]-3-yl)-5-chloropyrimidin-2-yl)amino)cyclohexane-1-carbonyl)piperidin-4-yl)ethyl)piperidin-4-yl)-1-oxoisoindolin-2-yl)piperidine-2,6-dione (Compound 62). To a solution of 3-(1-oxo-5-(1-(2-(piperidin-4-yl)ethyl)piperidin-4-yl)isoindolin-2-yl)piperidine-2,6-dione (091-3) (365 mg, 661 μmol, 1.00 eq, TFA), DIEA (427 mg, 3.30 mmol, 575 μL, 5.00 eq) and cis-3-((4-([1,1′-biphenyl]-3-yl)-5-chloropyrimidin-2-yl)amino)cyclohexane-1-carboxylic acid (294 mg, 661 μmol, 1.00 eq, HCl) (prepared by methods analogous to those shown in Example 22) in DMF (4 mL) were added HATU (301 mg, 793 μmol, 1.20 eq) and HOAt (450 mg, 330 μmol, 46.2 μL, 0.500 eq) at 25 °C. The mixture was stirred at 25 °C for 1 h. LCMS showed 3-(1-oxo-5-(1-(2-(piperidin-4-yl)ethyl)piperidin-4-yl)isoindolin-2-yl)piperidine-2,6-dione (091-3) was consumed completely and the desired mass was detected. Water (10 mL) was added to the mixture at 25 °C. Then the mixture was extracted with EtOAc (5 mL*3). The combined organic layers were dried over anhydrous sodium sulfate, filtered and the filtrate was concentrated under reduced pressure to give a residue. The residue was purified by prep-HPLC (column: Phenomenex Luna C18 75*30 mm*3 um; mobile phase: [water (FA)-ACN]; B %: 30%-65%, 8 min) to give the title compound (Compound 62) (41.0 mg, 48.3 μmol, 7.31% yield, 97.6% purity) as a white solid. LCMS (ES+): m/z 828.3 [M+H]+.

### Example 92. Synthesis of N-(3-(4-(4-((4-([1,1′-biphenyl]-3-yl)-5-chloropyrimidin-2-yl)amino)piperidine-1-carbonyl)piperidin-1-yl)-3-oxopropyl)-2-((2-(2,6-dioxopiperidin-3-yl)-1-oxoisoindolin-5-yl)oxy)acetamide (Compound 67)

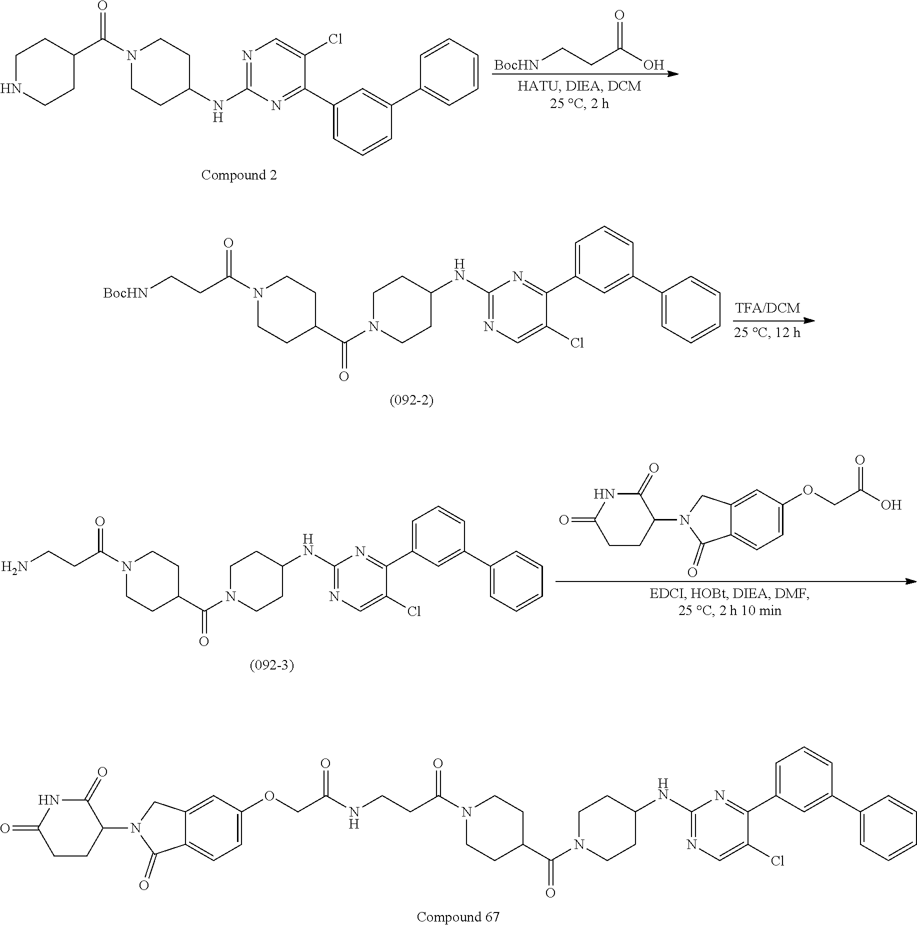 [SMILES: O=C(C1CCNCC1)N1CCC(Nc2ncc(Cl)c(-c3cccc(-c4ccccc4)c3)n2)CC1 ; CC(C)(C)OC(=O)NCCC(=O)O ; CC(C)(C)OC(=O)NCCC(=O)N1CCC(C(=O)N2CCC(Nc3ncc(Cl)c(-c4cccc(-c5ccccc5)c4)n3)CC2)CC1 ; NCCC(=O)N1CCC(C(=O)N2CCC(Nc3ncc(Cl)c(-c4cccc(-c5ccccc5)c4)n3)CC2)CC1 ; O=C(O)COc1ccc2c(c1)CN(C1CCC(=O)NC1=O)C2=O ; O=C(COc1ccc2c(c1)CN(C1CCC(=O)NC1=O)C2=O)NCCC(=O)N1CCC(C(=O)N2CCC(Nc3ncc(Cl)c(-c4cccc(-c5ccccc5)c4)n3)CC2)CC1]

Synthesis of tert-butyl (3-(4-(4-((4-([1,1′-biphenyl]-3-yl)-5-chloropyrimidin-2-yl)amino)piperidine-1-carbonyl)piperidin-1-yl)-3-oxopropyl)carbamate (092-2). To a solution of (4-((4-([1,1′-biphenyl]-3-yl)-5-chloropyrimidin-2-yl)amino)piperidin-1-yl)(piperidin-4-yl)methanone (Compound 2) (300 mg, 630 μmol, 1.00 eq) and 3-(tert-butoxycarbonylamino)propanoic acid (119 mg, 630 μmol, 1.00 eq) in DCM (5 mL) were added HATU (359 mg, 945 μmol, 1.50 eq) and DIEA (489 mg, 3.78 mmol, 659 μL, 6.00 eq) at 25 °C. The mixture was stirred at 25 °C for 2 h. LCMS showed (4-((4-([1,1′-biphenyl]-3-yl)-5-chloropyrimidin-2-yl)amino)piperidin-1-yl)(piperidin-4-yl)methanone (Compound 2) was consumed completely and the desired mass was detected. The reaction mixture was concentrated under reduced pressure to remove solvent. The residue was purified by prep-TLC (SiO₂, Petroleum ether:Ethyl acetate=1:3) to give the title compound (092-2) (480 mg, 563 μmol, 89.3% yield, 75.9% purity) as a yellow oil. LCMS (ES+): m/z 547.3 [M+H−100]+.

Synthesis of 1-(4-(4-((4-([1,1′-biphenyl]-3-yl)-5-chloropyrimidin-2-yl)amino)piperidine-1-carbonyl)piperidin-1-yl)-3-aminopropan-1-one (092-3). To a solution of tert-butyl (3-(4-(4-((4-([1,1′-biphenyl]-3-yl)-5-chloropyrimidin-2-yl)amino)piperidine-1-carbonyl)piperidin-1-yl)-3-oxopropyl)carbamate (092-2) (150 mg, 232 μmol, 1.00 eq) in DCM (3 mL) was added TFA (26.4 mg, 232 μmol, 17.2 μL, 1.00 eq) at 25 °C. The mixture was stirred at 25 °C for 12 h. LCMS showed tert-butyl (3-(4-(4-((4-([1,1′-biphenyl]-3-yl)-5-chloropyrimidin-2-yl)amino)piperidine-1-carbonyl)piperidin-1-yl)-3-oxopropyl)carbamate (092-2) was consumed completely and the desired mass was detected. The reaction mixture was concentrated under reduced pressure to remove solvent to give the title compound (092-3) (150 mg, crude, TFA) as a yellow oil. LCMS (ES+): m/z 547.3 [M+H]+.

Synthesis of N-(3-(4-(4-((4-([1,1′-biphenyl]-3-yl)-5-chloropyrimidin-2-yl)amino)piperidine-1-carbonyl)piperidin-1-yl)-3-oxopropyl)-2-((2-(2,6-dioxopiperidin-3-yl)-1-oxoisoindolin-5-yl)oxy)acetamide (Compound 67). To a solution of 2-[2-(2,6-dioxo-3-piperidyl)-1-oxo-isoindolin-5-yl]oxyacetic acid (60.0 mg, 189 μmol, 1.00 eq) (as prepared in Example 66) in DMF (4 mL) were added EDCI (54.2 mg, 283 μmol, 1.50 eq) and DIEA (73.1 mg, 566 μmol, 98.5 μL, 3.00 eq) at 25 °C. The mixture was stirred at 25 °C for 5 min. HOBt (38.2 mg, 283 μmol, 1.50 eq) added to the mixture at 25 °C. The mixture was stirred at 25 °C for 5 min. Then 1-(4-(4-((4-([1,1′-biphenyl]-3-yl)-5-chloropyrimidin-2-yl)amino)piperidine-1-carbonyl)piperidin-1-yl)-3-aminopropan-1-one (092-3) (150 mg, 226 μmol, 1.20 eq, TFA) in DMF (1 mL) added to the mixture at 25 °C. The mixture was stirred at 25 °C for 2 h. LCMS showed 1-(4-(4-((4-([1,1′-biphenyl]-3-yl)-5-chloropyrimidin-2-yl)amino)piperidine-1-carbonyl)piperidin-1-yl)-3-aminopropan-1-one (092-3) was consumed completely and the desired mass was detected. Water (4 mL) was added to the mixture at 25 °C. Then the reaction mixture was extracted with EtOAc (3 mL*3). The organic layers were dried over anhydrous sodium sulfate, filtered and the filtrate was concentrated under reduced pressure to give a residue. The residue was purified by prep-HPLC (column: Phenomenex Luna C18 75*30 mm*3 um; mobile phase: [water (FA)-ACN]; B %: 40%-80%, 8 min) to give the title compound (Compound 67) (20.4 mg, 24.1 μmol, 12.8% yield, 100% purity) as a white solid. LCMS (ES+): m/z 847.2 [M+H]+

### Example 93. Synthesis of cis-3-((4-(1-cyclohexyl-1H-pyrazol-3-yl)-5-fluoropyrimidin-2-yl)amino)cyclohexane-1-carboxamide (Compound 113)

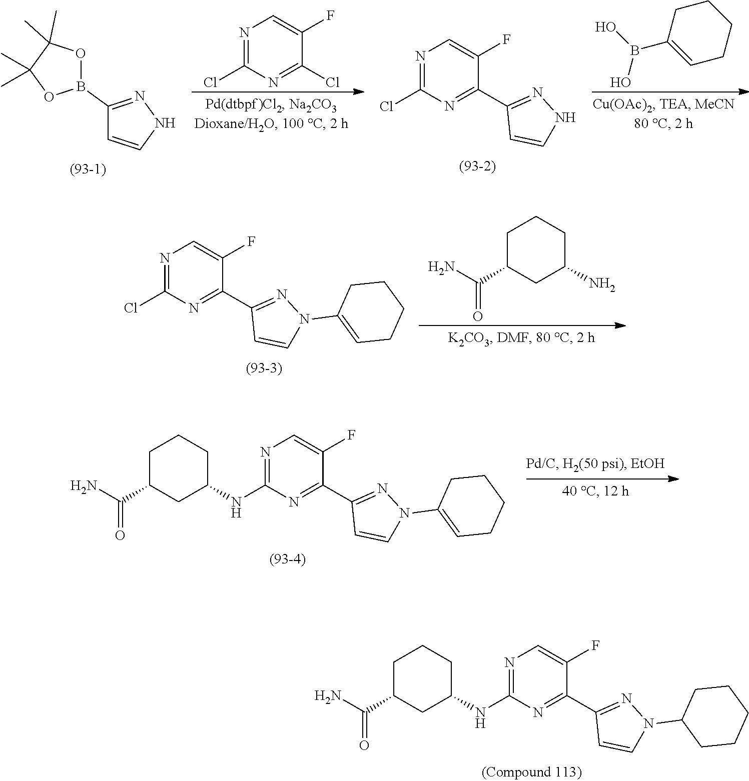

Synthesis of 2-chloro-5-fluoro-4-(1H-pyrazol-3-yl)pyrimidine (93-2). To a solution of 3-(4,4,5,5-tetramethyl-1,3,2-dioxaborolan-2-yl)-1H-pyrazole (93-1) (4.00 g, 20.6 mmol, 1.00 eq) in dioxane (10 mL) and H₂O (2 mL) was added Na₂CO₃ (3.28 g, 30.9 mmol, 1.50 eq), 2,4-dichloro-5-fluoro-pyrimidine (4.47 g, 26.8 mmol, 1.30 eq) and ditert-butyl(cyclopentyl)phosphane; dichloropalladium; iron (2.02 g, 3.09 mmol, 0.150 eq) at 25 °C under N₂. The mixture was stirred at 100 °C for 2 h under N₂. LC-MS showed 3-(4,4,5,5-tetramethyl-1,3,2-dioxaborolan-2-yl)-1H-pyrazole (93-1) was consumed completely and the desired mass was detected. The reaction diluted with H₂O 100 mL and combined organic layers were extracted with EtOAc (100 mL*3), dried over Na₂SO₄, filtered and concentrated under reduced pressure. The residue was purified by column chromatography (SiO₂, Petroleum ether:Ethyl acetate=1:0 to 3:1) to give the title compound (93-2) (1.50 g, 6.45 mmol, 31.2% yield, 85.3% purity) as a brown solid. LCMS (ES⁺): m/z 199.0 [M+H]⁺.

Synthesis of 2-chloro-4-(1-(cyclohex-1-en-1-yl)-1H-pyrazol-3-yl)-5-fluoropyrimidine (93-3). To a solution of 2-chloro-5-fluoro-4-(1H-pyrazol-3-yl)pyrimidine (93-2) (1.00 g, 5.04 mmol, 1.00 eq) in MeCN (30 mL) was added Cu(OAc)₂ (182 mg, 1.01 mmol, 0.200 eq), cyclohexen-1-ylboronic acid (951 mg, 7.55 mmol, 1.50 eq), TEA (1.53 g, 15.1 mmol, 2.10 mL, 3.00 eq) and Na₂CO₃ (800 mg, 7.55 mmol, 1.50 eq) at 25 °C. The mixture was stirred at 80 °C for 2 h. LC-MS showed 2-chloro-5-fluoro-4-(1H-pyrazol-3-yl)pyrimidine (93-2) was consumed completely and the desired mass was detected. The reaction diluted with H₂O 50 mL and combined organic layers were extracted with EtOAc (50 mL*3), dried over Na₂SO₄, filtered and concentrated under reduced pressure. The residue was purified by column chromatography (SiO₂, Petroleum ether:Ethyl acetate=1:0 to 3:1) to give the title compound (93-3) (200 mg, 537 μmol, 10.6% yield, 74.9% purity) as a yellow solid. LCMS (ES⁺): m/z 279.0 [M+H]⁺.

Synthesis of cis-3-((4-(1-(cyclohex-1-en-1-yl)-1H-pyrazol-3-yl)-5-fluoropyrimidin-2-yl)amino)cyclohexane-1-carboxamide (93-4). To a solution of 2-chloro-4-(1-(cyclohex-1-en-1-yl)-1H-pyrazol-3-yl)-5-fluoropyrimidine (93-3) (150 mg, 538 μmol, 1.00 eq) in DMF (2 mL) was added K₂CO₃ (74.3 mg, 538 μmol, 1.00 eq), cis-3-aminocyclohexane-1-carboxamide (229 mg, 1.61 mmol, 3.00 eq) at 25 °C. The mixture was stirred at 80 °C for 2 h. LC-MS showed 2-chloro-4-(1-(cyclohex-1-en-1-yl)-1H-pyrazol-3-yl)-5-fluoropyrimidine (93-3) was consumed completely and the desired mass was detected. The reaction diluted with H₂O 30 mL and combined organic layers were extracted with EtOAc (30 mL*3), dried over Na₂SO₄, filtered and concentrated under reduced pressure. The residue was purified by column chromatography by prep-TLC (SiO₂, Petroleum ether:Ethyl acetate=1:1) to give the title compound (93-4) (100 mg, 219 μmol, 40.7% yield, 84.3% purity) as a yellow oil. LCMS (ES⁺): m/z 385.1 [M+H]⁺.

Synthesis of cis-3-((4-(1-cyclohexyl-1H-pyrazol-3-yl)-5-fluoropyrimidin-2-yl)amino)cyclohexane-1-carboxamide (Compound 113). To a solution of cis-3-((4-(1-(cyclohex-1-en-1-yl)-1H-pyrazol-3-yl)-5-fluoropyrimidin-2-yl)amino)cyclohexane-1-carboxamide (93-4) (60.0 mg, 156 μmol, 1.00 eq) in EtOH (2 mL) was added 10% Pd/C (33.2 mg, 156 μmol, 50.0% purity, 1.00 eq) at 25 °C. The mixture was stirred at 40 °C under H₂ (50 Psi) for 12 h. LC-MS showed cis-3-((4-(1-(cyclohex-1-en-1-yl)-1H-pyrazol-3-yl)-5-fluoropyrimidin-2-yl)amino)cyclohexane-1-carboxamide (93-4) was consumed completely and the desired mass was detected. The reaction concentrated under reduced pressure. The residue was purified by prep-HPLC (column: Phenomenex luna C18 100*40 mm*3 um; mobile phase: [H₂O (0.2% FA)-ACN]; gradient: 30%-60% B over 8.0 min) to give the title compound (Compound 113) (1.50 mg, 3.88 μmol, 2.49% yield, 100% purity) as a brown solid. LCMS (ES⁺): m/z 387.1 [M+H]⁺.

### Example 94. Synthesis of cis-3-((5-fluoro-4-(3-(2-oxo-1,2-dihydropyridin-3-yl)phenyl)pyrimidin-2-yl)amino)cyclohexane-1-carboxamide (Compound 150)

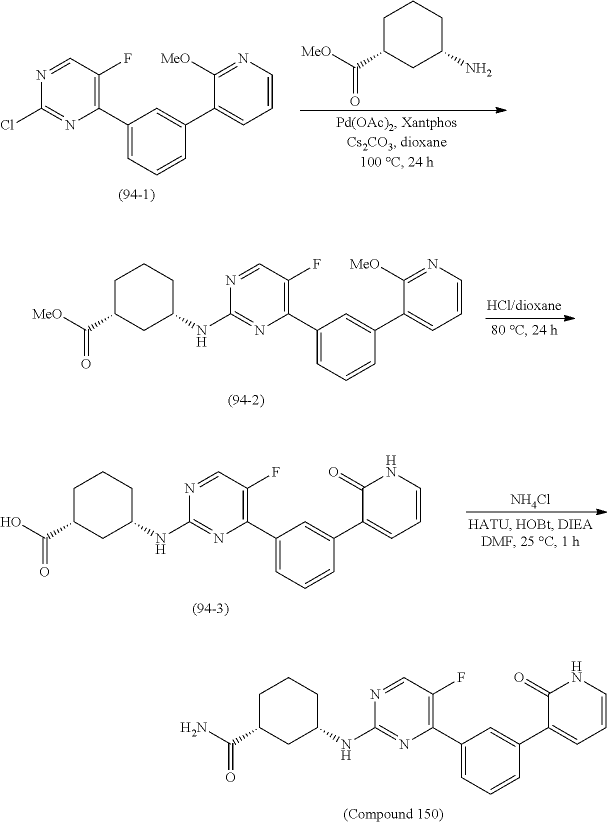 [SMILES: COc1ncccc1-c1cccc(-c2nc(Cl)ncc2F)c1 ; COC(=O)[C@@H]1CCC[C@H](N)C1 ; COC(=O)[C@@H]1CCC[C@H](Nc2ncc(F)c(-c3cccc(-c4cccnc4OC)c3)n2)C1 ; O=C(O)[C@@H]1CCC[C@H](Nc2ncc(F)c(-c3cccc(-c4ccc[nH]c4=O)c3)n2)C1 ; "NH4Cl" ; NC(=O)[C@@H]1CCC[C@H](Nc2ncc(F)c(-c3cccc(-c4ccc[nH]c4=O)c3)n2)C1]

Synthesis of cis-methyl 3-((5-fluoro-4-(3-(2-methoxypyridin-3-yl)phenyl)pyrimidin-2-yl)amino)cyclohexane-1-carboxylate (94-2). To a solution of 2-chloro-5-fluoro-4-(3-(2-methoxypyridin-3-yl)phenyl)pyrimidine (94-1) (350 mg, 1.11 mmol, 1.00 eq) and cis-methyl 3-aminocyclohexane-1-carboxylate (522 mg, 3.33 mmol, 3.00 eq) in dioxane (10 mL) were added Cs₂CO₃ (722 mg, 2.22 mmol, 2.00 eq), Pd(OAc)₂ (24.9 mg, 111 mol, 0.100 eq) and Xantphos (64.1 mg, 111 μmol, 0.100 eq) at 25 °C under N₂ atmosphere. The mixture was stirred at 100 °C for 12 h. LCMS showed 2-chloro-5-fluoro-4-(3-(2-methoxypyridin-3-yl)phenyl)pyrimidine (94-1) was remained mostly and the desired mass was detected, the mixture was stirred at 100 °C for another 12 h under N₂ atmosphere. LCMS showed 2-chloro-5-fluoro-4-(3-(2-methoxypyridin-3-yl)phenyl)pyrimidine (94-1) was consumed completely and the desired mass was detected. The mixture was diluted with H₂O (10 mL) and then extracted with EtOAc (10 mL*3). The combined organic layers were dried over Na₂SO₄, filtered and the filtrate was concentrated under reduced pressure. The mixture was purified by prep-TLC (SiO₂, PE/EtOAc=0/1) to give the title compound (94-2) (300 mg, 687 μmol, 62.00% yield) as a white solid. LCMS (ES⁺): m/z 437.1 [M+H]⁺.

Synthesis of cis-3-((5-fluoro-4-(3-(2-oxo-1,2-dihydropyridin-3-yl)phenyl)pyrimidin-2-yl)amino)cyclohexane-1-carboxylic acid (94-3). A solution of cis-methyl 3-((5-fluoro-4-(3-(2-methoxypyridin-3-yl)phenyl)pyrimidin-2-yl)amino)cyclohexane-1-carboxylate (94-2) (180 mg, 412 μmol, 1.00 eq) in HCl/dioxane (12 mL) was stirred at 80 °C for 12 h. LCMS showed cis-methyl 3-((5-fluoro-4-(3-(2-methoxypyridin-3-yl)phenyl)pyrimidin-2-yl)amino)cyclohexane-1-carboxylate (94-2) was remained mostly and the desired mass was detected, the mixture was stirred at 80 °C for another 12 h. LCMS showed cis-methyl 3-((5-fluoro-4-(3-(2-methoxypyridin-3-yl)phenyl)pyrimidin-2-yl)amino)cyclohexane-1-carboxylate (94-2) was consumed completely and the desired mass was detected. The mixture was concentrated under reduced pressure to give the title compound (94-3) (160 mg, crude) as a white solid. LCMS (ES+): m/z 409.1 [M+H]⁺.

Synthesis of cis-3-((5-fluoro-4-(3-(2-oxo-1,2-dihydropyridin-3-yl)phenyl)pyrimidin-2-yl)amino)cyclohexane-1-carboxamide (Compound 150). To a solution of cis-3-((5-fluoro-4-(3-(2-oxo-1,2-dihydropyridin-3-yl)phenyl)pyrimidin-2-yl)amino)cyclohexane-1-carboxylic acid (94-3) (152 mg, 372 μmol, 1.00 eq) in DMF (5 mL) was added HOBt (25.1 mg, 186 μmol, 0.500 eq), HATU (170 mg, 447 μmol, 1.20 eq), DIEA (96.2 mg, 744 μmol, 130 μL, 2.00 eq) and NH₄Cl (79.6 mg, 1.49 mmol, 4.00 eq) at 25 °C, the mixture was stirred at 25 °C for 1 h. LCMS showed cis-3-((5-fluoro-4-(3-(2-oxo-1,2-dihydropyridin-3-yl)phenyl)pyrimidin-2-yl)amino)cyclohexane-1-carboxylic acid (94-3) was consumed completely and the desired mass was detected. The mixture was diluted with H₂O (5 mL) and then extracted with EtOAc (5 mL*3). The combined organic layers were dried over Na₂SO₄, filtered and the filtrate was concentrated under reduced pressure. The residue was purified by acidic prep-HPLC (column: Phenomenex luna C18 100*40 mm*3 um; mobile phase: [H₂O (0.2% FA)-ACN]; gradient: 5%-50% B over 8.0 min) to give the title compound (Compound 150) (76.7 mg, 188 μmol, 50.6% yield) as a white solid. LCMS (ES⁺): m/z 408.1 [M+H]⁺.

### Example 95. Synthesis of (1R,3S)-3-((5-fluoro-4-(3-(2-oxo-1,2-dihydropyridin-3-yl)phenyl)pyrimidin-2-yl)amino)cyclohexane-1-carboxamide (Compound 204)

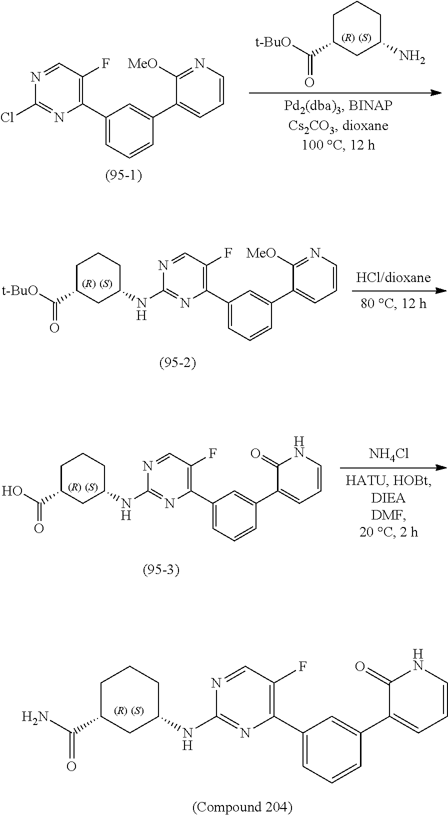

Synthesis of tert-butyl (1R,3S)-3-((5-fluoro-4-(3-(2-methoxypyridin-3-yl)phenyl)pyrimidin-2-yl)amino)cyclohexane-1-carboxylate (95-2). To a solution of 2-chloro-5-fluoro-4-(3-(2-methoxypyridin-3-yl)phenyl)pyrimidine (95-1) (300 mg, 950 μmol, 1.00 eq) and tert-butyl (1R,3S)-3-aminocyclohexane-1-carboxylate (341 mg, 1.71 mmol, 1.80 eq) in dioxane (5 mL) were added Pd₂(dba)₃ (43.5 mg, 47.5 μmol, 0.0500 eq), BINAP (53.3 mg, 85.5 μmol, 0.0900 eq) and Cs₂CO₃ (929 mg, 2.85 mmol, 3.00 eq) at 20 °C under N₂ atmosphere. The mixture was stirred at 100 °C for 12 h under N₂. LCMS showed 2-chloro-5-fluoro-4-(3-(2-methoxypyridin-3-yl)phenyl)pyrimidine (95-1) was consumed and the desired mass was detected. To the mixture was added H₂O (10 mL) and the mixture was extracted with EtOAc (8 mL*3). The combined organic layers were washed with brine (10 mL*3), dried over Na₂SO₄, filtered and the filtrate was concentrated under reduced pressure. The mixture was purified by prep-TLC (SiO₂, PE/EtOAc=1/1) to give the title compound (95-2) (400 mg, 836 μmol, 88.0% yield) as yellow oil. LCMS (ES⁺): m/z 479.3[M+H]⁺.

Synthesis of (1R,3S)-3-((5-fluoro-4-(3-(2-oxo-1,2-dihydropyridin-3-yl)phenyl)pyrimidin-2-yl)amino)cyclohexane-1-carboxylic acid (95-3). A solution of tert-butyl (1R,3S)-3-((5-fluoro-4-(3-(2-methoxypyridin-3-yl)phenyl)pyrimidin-2-yl)amino)cyclohexane-1-carboxylate (95-2) (400 mg, 836 μmol, 1.00 eq) in HCl/dioxane (10 mL) was stirred at 80 °C for 12 h. LCMS showed tert-butyl (1R,3S)-3-((5-fluoro-4-(3-(2-methoxypyridin-3-yl)phenyl)pyrimidin-2-yl)amino)cyclohexane-1-carboxylate (95-2) was consumed and the desired mass was detected. The mixture was concentrated under reduced pressure to give a residue. Water (10 mL) was added to the residue, then the aqueous was alkalified by LiOH·H₂O (1 M) to adjust pH=8-9 and washed with EtOAc (5 mL*3). Then the aqueous was acidified by HCl (1 M) to adjust pH=4-5 and extracted with EtOAc (5 mL*3). The above organic layers were dried over Na₂SO₄, filtered and the filtrate was concentrated under reduced pressure to give the title compound (95-3) (400 mg, crude) as yellow solid. LCMS (ES⁺): m/z 409.2 [M+H]⁺.

Synthesis of (1R,3S)-3-((5-fluoro-4-(3-(2-oxo-1,2-dihydropyridin-3-yl)phenyl)pyrimidin-2-yl)amino)cyclohexane-1-carboxamide (Compound 204). To a solution of (1R,3S)-3-((5-fluoro-4-(3-(2-oxo-1,2-dihydropyridin-3-yl)phenyl)pyrimidin-2-yl)amino)cyclohexane-1-carboxylic acid (95-3) (400 mg, 979 μmol, 1.00 eq) and NH₄Cl (210 mg, 3.92 mmol, 4.00 eq) in DMF (2 mL) were added DIEA (253 mg, 1.96 mmol, 341 μL, 2.00 eq), HATU (447 mg, 1.18 mmol, 1.2 eq) and HOBt (66.2 mg, 490 μmol, 0.500 eq) at 20 °C. The resulting mixture was stirred at 20 °C for 2 h. LCMS showed (1R,3S)-3-((5-fluoro-4-(3-(2-oxo-1,2-dihydropyridin-3-yl)phenyl)pyrimidin-2-yl)amino)cyclohexane-1-carboxylic acid (95-3) was consumed and the desired mass was detected. The mixture was concentrated under reduced pressure to give a residue. The residue was purified by acidic prep-HPLC (column: Phenomenex luna C18 100*40 mm*3 um; mobile phase: [H₂O (0.2% FA)-ACN]; gradient: 20%-55% B over 8.0 min) to give the title compound (Compound 204) (253 mg, 621 μmol, 63.4% yield, 100% purity) as a yellow solid. LCMS (ES⁺): m/z 408.1 [M+H]⁺.

### Example 96. Synthesis N-(trans-4-((5-fluoro-4-(3-(2-oxo-1,2-dihydropyridin-3-yl)phenyl)pyrimidin-2-yl)amino)cyclohexyl)acetamide (Compound 255)

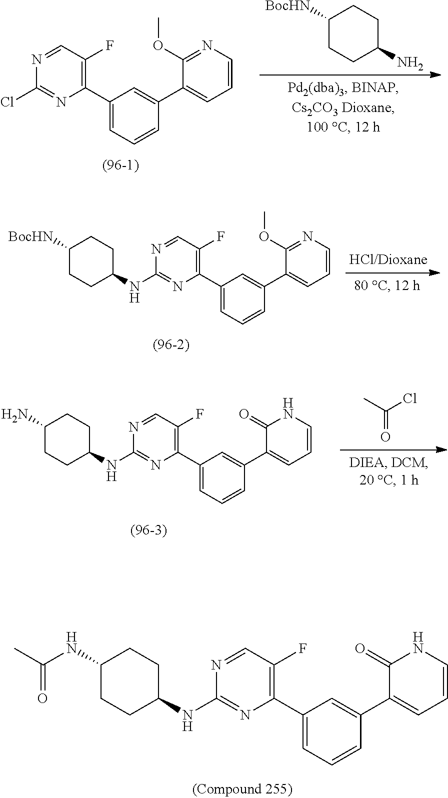

Synthesis of trans-tert-butyl (4-((5-fluoro-4-(3-(2-methoxypyridin-3-yl)phenyl)pyrimidin-2-yl)amino)cyclohexyl)carbamate (96-2). A mixture of 2-chloro-5-fluoro-4-(3-(2-methoxypyridin-3-yl)phenyl)pyrimidine (96-1) (5.00 g, 15.8 mmol, 1.00 eq), tert-butyl (trans-4-aminocyclohexyl)carbamate (5.09 g, 23.7 mmol, 1.50 eq), Pd₂(dba)₃ (1.45 g, 1.58 mmol, 0.100 eq), BINAP (986 mg, 1.58 mmol, 0.100 eq) and Cs₂CO₃ (10.3 g, 31.6 mmol, 2.00 eq) in 1,4-dioxane (10 mL) was degassed and purged with N₂ at 20 °C for 3 times, and then the mixture was stirred at 100 °C for 12 h under N₂ atmosphere. LC-MS showed the desired mass was detected. The reaction mixture was quenched by addition H₂O (10 mL), and then diluted with H₂O (40 mL) and extracted with EtOAc (50 mL*3). The combined organic layers were washed with sat.aq. NaCl (10 mL), dried over Na₂SO₄, filtered and concentrated under reduced pressure. The residue was purified by column chromatography (SiO₂, Petroleum ether:Ethyl acetate=1:0 to 0:1) to give the title compound (96-2) (16.0 g, 23.3 mmol, 73.5% yield, 71.8% purity) as a yellow solid. LCMS (ES⁺): m/z 494.3 [M+H]⁺.

Synthesis of trans-3-(3-(2-((4-aminocyclohexyl)amino)-5-fluoropyrimidin-4-yl)phenyl)pyridin-2(1H)-one (96-3). A solution of trans-tert-butyl (4-((5-fluoro-4-(3-(2-methoxypyridin-3-yl)phenyl)pyrimidin-2-yl)amino)cyclohexyl)carbamate (96-2) (3.00 g, 6.08 mmol, 1.00 eq) in HCl/dioxane (4 M, 30 mL, 19.7 eq) was stirred at 80 °C for 12 h. LC-MS showed the desired mass was detected. The reaction mixture was concentrated under high vacuum to give the title compound (96-3) (2.60 g, crude, HCl) as a yellow solid. LCMS (ES⁺): m/z 380.3 [M+H]⁺.

Synthesis of N-(trans-4-((5-fluoro-4-(3-(2-oxo-1,2-dihydropyridin-3-yl)phenyl)pyrimidin-2-yl)amino)cyclohexyl)acetamide (Compound 255). To a solution of trans-3-(3-(2-((4-aminocyclohexyl)amino)-5-fluoropyrimidin-4-yl)phenyl)pyridin-2(1H)-one (96-3) (500 mg, 1.20 mmol, 1.00 eq, HCl) in DCM (0.5 mL) was added DIEA (310 mg, 2.40 mmol, 418 μL, 2.00 eq) and acetyl chloride (94.3 mg, 1.20 mmol, 85.4 μL, 1.00 eq) at 20 °C. The mixture was stirred at 20 °C for 1 h. LC-MS showed the desired mass was detected. The reaction mixture was quenched by addition H₂O 10 mL, and then diluted with H₂O 10 mL and extracted with EtOAc (10 mL*3). The combined organic layers were washed with sat.aq. NaCl (10 mL), dried over Na₂SO₄, filtered and concentrated under reduced pressure. The residue was purified by prep-HPLC (Phenomenex Luna C18 100*30 mm*3 um; mobile phase: [H₂O (0.2% FA)-ACN]; gradient: 20%-50% B over 8.0 min) to give the title compound (Compound 255) (112 mg, 266 μmol, 22.1% yield, 99.4% purity) as a white solid. LCMS (ES⁺): m/z 422.2 [M+H]⁺.

### Example 97. Synthesis trans-(1r,4r)-4-((5-fluoro-4-(3-(2-oxo-1,2-dihydropyridin-3-yl)phenyl)pyrimidin-2-yl)amino)cyclohexane-1-carboxamide (Compound 254)

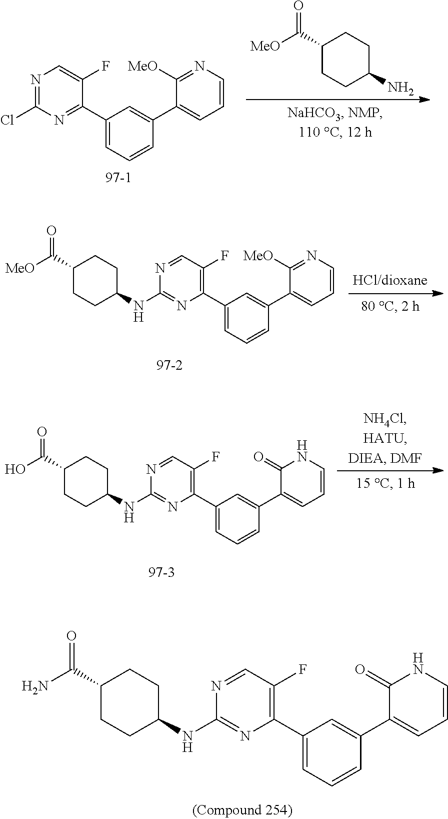 [SMILES: COc1ncccc1-c1cccc(-c2nc(Cl)ncc2F)c1 ; COC(=O)[C@H]1CC[C@H](N)CC1 ; COc1ncccc1-c1cccc(-c2nc(N[C@H]3CC[C@H](C(=O)OC)CC3)ncc2F)c1 ; O=c1[nH]cccc1-c1cccc(-c2nc(N[C@H]3CC[C@H](C(=O)O)CC3)ncc2F)c1 ; "NH4Cl,HATU, DIEA, DMF" ; NC(=O)[C@H]1CC[C@H](Nc2ncc(F)c(-c3cccc(-c4ccc[nH]c4=O)c3)n2)CC1]

Synthesis of trans-methyl (1r,4r)-4-((5-fluoro-4-(3-(2-methoxypyridin-3-yl)phenyl)pyrimidin-2-yl)amino)cyclohexane-1-carboxylate (97-2). To a solution of 2-chloro-5-fluoro-4-(3-(2-methoxypyridin-3-yl)phenyl)pyrimidine (97-1) (2.00 g, 6.33 mmol, 1.00 eq) in NMP (100 mL) were added NaHCO₃ (2.66 g, 31.7 mmol, 1.23 mL, 5.00 eq) and trans-methyl (1r,4r)-4-aminocyclohexane-1-carboxylate (3.07 g, 15.8 mmol, 2.50 eq, HCl) at 15 °C. The mixture was stirred at 110 °C for 12 h. LC-MS showed 2-chloro-5-fluoro-4-(3-(2-methoxypyridin-3-yl)phenyl)pyrimidine (97-1) was consumed completely, and the desired mass was detected. The reaction mixture was diluted with water 100 mL and extracted with EtOAc (100 mL*3). The combined organic layers were washed with brine (100 mL*3), dried over Na₂SO₄, filtered and the filtrate was concentrated under reduced pressure. The residue was purified by column chromatography (SiO₂, Petroleum ether/Ethyl acetate=3/1 to 1/1) to give title compound (97-2) (2.20 g, 5.04 mmol, 79.6% yield) as a yellow solid. LCMS (ES⁺): m/z 437.2 [M+H]⁺.

Synthesis of trans-(1r,4r)-4-((5-fluoro-4-(3-(2-oxo-1,2-dihydropyridin-3-yl)phenyl)pyrimidin-2-yl)amino)cyclohexane-1-carboxylic acid (97-3). To a solution of trans-methyl (1r,4r)-4-((5-fluoro-4-(3-(2-methoxypyridin-3-yl)phenyl)pyrimidin-2-yl)amino)cyclohexane-1-carboxylate (97-2) (2.00 g, 4.58 mmol, 1.00 eq) in dioxane (20 mL) was added HCl (12 M, 20 mL, 52.4 eq) at 15 °C. The mixture was stirred at 80 °C for 2 h. LC-MS showed trans-methyl (1r,4r)-4-((5-fluoro-4-(3-(2-methoxypyridin-3-yl)phenyl)pyrimidin-2-yl)amino)cyclohexane-1-carboxylate (97-2) was consumed completely and the desired mass was detected. The mixture was concentrated under reduced pressure to give title compound (97-3) (2.00 g, crude) as a yellow oil. LCMS (ES⁺): m/z 409.2 [M+H]⁺.

Synthesis of trans-(1r,4r)-4-((5-fluoro-4-(3-(2-oxo-1,2-dihydropyridin-3-yl)phenyl)pyrimidin-2-yl)amino)cyclohexane-1-carboxamide (Compound 254). To a solution of trans-(1r,4r)-4-((5-fluoro-4-(3-(2-oxo-1,2-dihydropyridin-3-yl)phenyl)pyrimidin-2-yl)amino)cyclohexane-1-carboxylic acid (97-3) (500 mg, 1.22 mmol, 1.00 eq) in DMF were added DIEA (475 mg, 3.67 mmol, 640 μL, 3.00 eq), NH₄Cl (98.2 mg, 1.84 mmol, 1.50 eq) and HATU (698 mg, 1.84 mmol, 1.50 eq) at 15 °C. The mixture was stirred at 15 °C for 1 h. LC-MS showed trans-(1r,4r)-4-((5-fluoro-4-(3-(2-oxo-1,2-dihydropyridin-3-yl)phenyl)pyrimidin-2-yl)amino)cyclohexane-1-carboxylic acid (97-3) was consumed completely and the desired mass was detected. The reaction mixture was concentrated under reduced pressure to give a residue. The crude product was triturated with EtOAc (10 ml) at 15 °C for 30 min to give title compound (Compound 254) (74.1 mg, 171 μmol, 14.0% yield, 94.0% purity) as a white solid. LCMS (ES⁺): m/z 408.0 [M+H]⁺.

### Example 98. Synthesis of (1R,3S)-3-((5-fluoro-4-(3-(2-oxo-1,2-dihydropyridin-3-yl)phenyl)pyrimidin-2-yl)amino)cyclopentane-1-carboxamide (Compound 223)

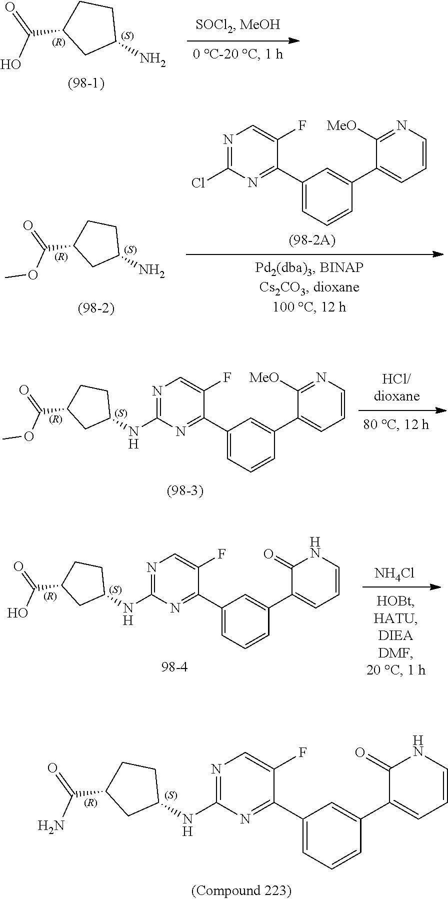

Synthesis of methyl (1R,3S)-3-aminocyclopentane-1-carboxylate (98-2). To a stirred solution of (1R,3S)-3-aminocyclopentane-1-carboxylic acid (98-1) (200 mg, 1.55 mmol, 1.00 eq) in MeOH (3 mL) was added dropwise SOCl₂ (368 mg, 3.10 mmol, 225 μL, 2.00 eq) at 0 °C. The resulting mixture was stirred at 20 °C for 1 h. LCMS showed the desired mass was detected. The mixture was concentrated under reduced pressure to give the title compound (98-2) (220 mg, crude) as a white solid. LCMS (ES⁺): m/z 144.1 [M+H]⁺.

Synthesis of methyl (1R,3S)-3-((5-fluoro-4-(3-(2-methoxypyridin-3-yl)phenyl)pyrimidin-2-yl)amino)cyclopentane-1-carboxylate (98-3). To a stirred solution of methyl (1R,3S)-3-aminocyclopentane-1-carboxylate (98-2) (136 mg, 950 μmol, 1.00 eq) and 2-chloro-5-fluoro-4-(3-(2-methoxypyridin-3-yl)phenyl)pyrimidine (300 mg, 950.18 μmol, 1.00 eq) in dioxane (3 mL) were added BINAP (29.58 mg, 47.51 μmol, 0.0500 eq), Cs₂CO₃ (928.76 mg, 2.85 mmol, 3.00 eq) and Pd₂(dba)₃ (43.51 mg, 47.51 μmol, 0.0500 eq) at 15 °C under N₂. The resulting mixture was stirred at 100 °C for 12 h under N₂. LCMS showed methyl (1R,3S)-3-aminocyclopentane-1-carboxylate (98-2) was consumed completely and the desired mass was detected. The mixture was diluted with H₂O (8 mL) and then extracted with EtOAc (8 mL*3). The combined organic layers were dried over Na₂SO₄, filtered and the filtrate was concentrated under reduced pressure. The mixture was purified prep-TLC (SiO₂, PE/EtOAc=0/1) to give the title compound (98-3) (250 mg, 592 μmol, 62.3% yield) as a yellow solid. LCMS (ES⁺): m/z 423.2 [M+H]⁺.

Synthesis of (1R,3S)-3-((5-fluoro-4-(3-(2-oxo-1,2-dihydropyridin-3-yl)phenyl)pyrimidin-2-yl)amino)cyclopentane-1-carboxylic acid (98-4). A solution of methyl (1R,3S)-3-((5-fluoro-4-(3-(2-methoxypyridin-3-yl)phenyl)pyrimidin-2-yl)amino)cyclopentane-1-carboxylate (98-3) (200 mg, 473.43 μmol, 1.00 eq) in HCl/dioxane (4 mL) was stirred at 80 °C for 12 h. LCMS showed methyl (1R,3S)-3-((5-fluoro-4-(3-(2-methoxypyridin-3-yl)phenyl)pyrimidin-2-yl)amino)cyclopentane-1-carboxylate (98-3) was consumed completely and the desired mass was detected. The mixture was concentrated under reduced pressure to give the title compound (98-4) (180 mg, crude) as a yellow solid. LCMS (ES⁺): m/z 395.2 [M+H]⁺.

Synthesis of (1R,3S)-3-((5-fluoro-4-(3-(2-oxo-1,2-dihydropyridin-3-yl)phenyl)pyrimidin-2-yl)amino)cyclopentane-1-carboxamide (Compound 223). To a solution of (1R,3S)-3-((5-fluoro-4-(3-(2-oxo-1,2-dihydropyridin-3-yl)phenyl)pyrimidin-2-yl)amino)cyclopentane-1-carboxylic acid (98-4) (106 mg, 269 μmol, 1.00 eq) in DMF (2 mL) was added HOBt (18.2 mg, 134 μmol, 0.500 eq), HATU (123 mg, 323 μmol, 1.20 eq) and DIEA (69.4 mg, 538 μmol, 93.6 μL, 2.00 eq) at 20 °C, then to the reaction was added NH₄Cl (57.5 mg, 1.08 mmol, 4.00 eq) at 20 °C, the mixture was stirred at 20 °C for 1 h. LCMS showed (1R,3S)-3-((5-fluoro-4-(3-(2-oxo-1,2-dihydropyridin-3-yl)phenyl)pyrimidin-2-yl)amino)cyclopentane-1-carboxylic acid (98-4) was consumed completely and the desired mass was detected. H₂O (3 mL) was added to the solution, the mixture was extracted with EtOAc (5 mL*3), then the combined organic layers were washed with brine (5 mL), dried over Na₂SO₄, filtered and the filtrate was concentrated under reduced pressure. The residue was purified by prep-HPLC (column: Phenomenex luna C18 100*40 mm*5 um; mobile phase: [H₂O (0.2% FA)-ACN]; gradient: 15%-55% B over 8.0 min) to give the title compound (Compound 223) (6.9 mg, 17.5 μmol, 6.53% yield, 100% purity) as a white solid. LCMS (ES⁺): m/z 394.1 [M+H]⁺.

### Example 99. Synthesis of (S)-3-(3-(2-((1-acetylpiperidin-3-yl)amino)-5-chloropyrimidin-4-yl)phenyl)pyridin-2(1H)-one (Compound 152)

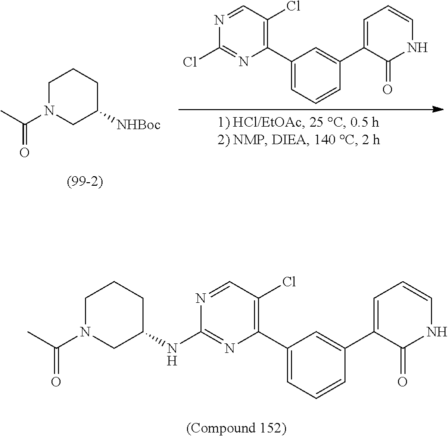 [SMILES: CC(=O)N1CCC[C@H](NC(=O)OC(C)(C)C)C1 ; O=c1[nH]cccc1-c1cccc(-c2nc(Cl)ncc2Cl)c1 ; CC(=O)N1CCC[C@H](Nc2ncc(Cl)c(-c3cccc(-c4ccc[nH]c4=O)c3)n2)C1]

Synthesis of(S)-3-(3-(2-((1-acetylpiperidin-3-yl)amino)-5-chloropyrimidin-4-yl)phenyl)pyridin-2(1H)-one (Compound 152). To a solution of tert-butyl (S)-(1-acetylpiperidin-3-yl)carbamate (99-2) (152.32 mg, 628.62 μmol, 1 eq) in HCl/EtOAc (5 mL) was stirred at 25 °C for 0.5 h. The reaction mixture was concentrated under reduced pressure to give a residue and then the residue in NMP (2 mL) was added 3-[3-(2,5-dichloropyrimidin-4-yl)phenyl]-1H-pyridin-2-one (200 mg, 628.62 μmol, 1 eq) and DIEA (406.22 mg, 3.14 mmol, 547.47 μL, 5 eq) at 25 °C the mixture was stirred at 140 °C for 2 h. LCMS showed 3-[3-(2,5-dichloropyrimidin-4-yl)phenyl]-1H-pyridin-2-one was consumed completely and desired mass was detected. The reaction did not work up, purified directly. The residue was purified by prep-HPLC (Neutral condition; column: Waters xbridge 150*25 mm 10 um; mobile phase: [water (NH₄HCO₃)-ACN]; gradient: 21%-51% B over 14 min) to give title compound (Compound 152) (41.2 mg, 94.70 μmol, 15.06% yield, 97.43% purity) as a white solid. LCMS (ES+): m/z 424.0 [M+H]⁺.

### Example 100. Synthesis of 3-(3-(2-((1-acetylpiperidin-4-yl)amino)-5-chloropyrimidin-4-yl)phenyl)pyridin-2(1H)-one (Compound 137)

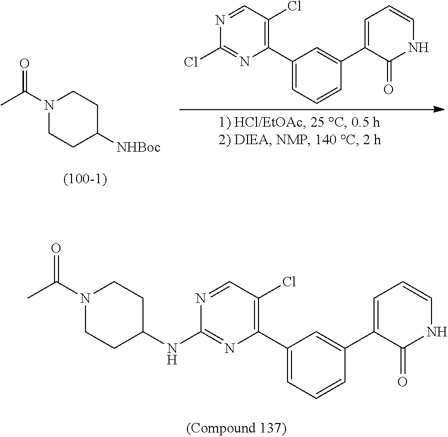 [SMILES: CC(=O)N1CCC(NC(=O)OC(C)(C)C)CC1 ; O=c1[nH]cccc1-c1cccc(-c2nc(Cl)ncc2Cl)c1 ; CC(=O)N1CCC(Nc2ncc(Cl)c(-c3cccc(-c4ccc[nH]c4=O)c3)n2)CC1]

Synthesis of 3-(3-(2-((1-acetylpiperidin-4-yl)amino)-5-chloropyrimidin-4-yl)phenyl)pyridin-2(1H)-one (Compound 137). To a solution of tert-butyl (1-acetylpiperidin-4-yl)carbamate (100-1) (152.32 mg, 628.62 μmol, 1 eq) in HCl/EtOAc (5 mL) was stirred at 25 °C for 0.5 h. The reaction mixture was concentrated under reduced pressure to give a residue and then the residue in NMP (2 mL) was added 3-[3-(2,5-dichloropyrimidin-4-yl)phenyl]-1H-pyridin-2-one (200 mg, 628.62 μmol, 1 eq) and DIEA (406.22 mg, 3.14 mmol, 547.47 μL, 5 eq) at 25 °C, the mixture was stirred at 140 °C for 2 h. LCMS showed 3-[3-(2,5-dichloropyrimidin-4-yl)phenyl]-1H-pyridin-2-one was consumed completely and desired mass was detected. The reaction did not work up, purified directly. The residue was purified by prep-HPLC (Neutral condition; column: Waters xbridge 150*25 mm 10 um; mobile phase: [water-ACN]; gradient: 21%-51% B over 14 min) to give title compound (Compound 137) (88.2.2 mg, 94.70 μmol, 15.06% yield, 97.43% purity) as a white solid. LCMS (ES+): m/z 424.0 [M+H]⁺.

### Example 101. Synthesis of cis-N-((1s,4s)-4-((5-chloro-4-(3-(2-oxo-1,2-dihydropyridin-3-yl)phenyl)pyrimidin-2-yl)amino)cyclohexyl)acetamide (Compound 153)

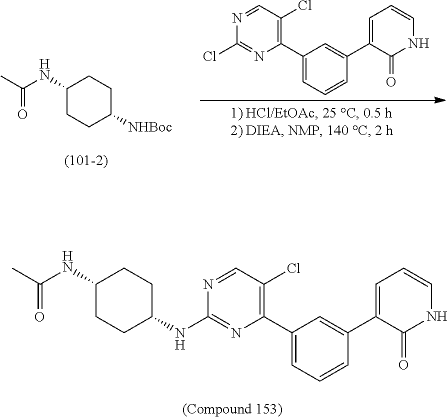 [SMILES: CC(=O)N[C@H]1CC[C@@H](NC(=O)OC(C)(C)C)CC1 ; O=c1[nH]cccc1-c1cccc(-c2nc(Cl)ncc2Cl)c1 ; CC(=O)N[C@H]1CC[C@@H](Nc2ncc(Cl)c(-c3cccc(-c4ccc[nH]c4=O)c3)n2)CC1]

Synthesis of cis-N-((1s,4s)-4-((5-chloro-4-(3-(2-oxo-1,2-dihydropyridin-3-yl)phenyl)pyrimidin-2-yl)amino)cyclohexyl)acetamide (Compound 153). To a solution of cis-tert-butyl ((1s,4s)-4-acetamidocyclohexyl)carbamate (101-2) (161.14 mg, 628.62 μmol, 1 eq) in HCl/EtOAc (5 mL) was stirred at 25 °C for 0.5 h. The reaction mixture was concentrated under reduced pressure to give a residue and then the residue in NMP (2 mL) was added 3-(3-(2,5-dichloropyrimidin-4-yl)phenyl)pyridin-2(1H)-one (Compound AA) (200 mg, 628.62 μmol, 1 eq) and DIEA (406.22 mg, 3.14 mmol, 547.47 μL, 5 eq) at 25 °C and the mixture was stirred at 140 °C for 2 h. LCMS showed 3-(3-(2,5-dichloropyrimidin-4-yl)phenyl)pyridin-2(1H)-one (Compound AA) was consumed completely and desired mass was detected. The residue was purified by prep-HPLC (FA condition; column: Phenomenex Luna C18 150*25 mm*10 um; mobile phase: [water (FA)-ACN]; gradient: 27%-57% B over 10 min) to give title compound (Compound 153) (37.6 mg, 82.73 μmol, 13.16% yield, 96.35% purity) as a white solid. LCMS (ES+): m/z 438.3 [M+H]⁺.

### Example 102. Synthesis of trans-N-((1r,4r)-4-((5-chloro-4-(3-(2-oxo-1,2-dihydropyridin-3-yl)phenyl)pyrimidin-2-yl)amino)cyclohexyl)acetamide (Compound 154)

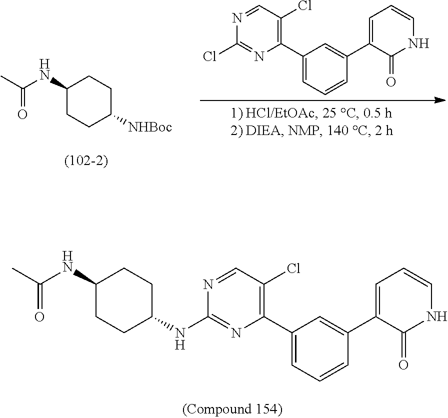 [SMILES: CC(=O)N[C@H]1CC[C@H](NC(=O)OC(C)(C)C)CC1 ; O=c1[nH]cccc1-c1cccc(-c2nc(Cl)ncc2Cl)c1 ; CC(=O)N[C@H]1CC[C@H](Nc2ncc(Cl)c(-c3cccc(-c4ccc[nH]c4=O)c3)n2)CC1]

Synthesis of trans-N-((1r,4r)-4-((5-chloro-4-(3-(2-oxo-1,2-dihydropyridin-3-yl)phenyl)pyrimidin-2-yl)amino)cyclohexyl)acetamide (Compound 154). To a solution of trans-tert-butyl ((1r,4r)-4-acetamidocyclohexyl)carbamate (102-2) (161.14 mg, 628.62 μmol, 1 eq) in HCl/EtOAc (5 mL) was stirred at 25 °C for 0.5 h. The reaction mixture was concentrated under reduced pressure to give a residue and then the residue in NMP (2 mL) was added 3-(3-(2,5-dichloropyrimidin-4-yl)phenyl)-1,2-dihydropyridin-2-ol (200 mg, 628.62 μmol, 1 eq) and DIEA (406.22 mg, 3.14 mmol, 547.47 μL, 5 eq) at 25 °C, the mixture was stirred at 140 °C for 2 h. LCMS showed 3-(3-(2,5-dichloropyrimidin-4-yl)phenyl)-1,2-dihydropyridin-2-ol was consumed completely and desired mass was detected. The residue was purified by prep-HPLC (FA condition; column: Phenomenex luna C18 150*25 mm*10 um; mobile phase: [water(FA)-ACN]; gradient: 27%-57% B over 10 min) to give title compound (Compound 154) (17.6 mg, 38.01 μmol, 6.05% yield, 94.58% purity) as a white solid. LCMS (ES⁺): m/z 438.2 [M+H]⁺.

### Example 103. Synthesis of trans-(1r,4r)-4-((5-chloro-4-(3-(2-oxo-1,2-dihydropyridin-3-yl)phenyl)pyrimidin-2-yl)amino)-N-methylcyclohexane-1-carboxamide (Compound 155)

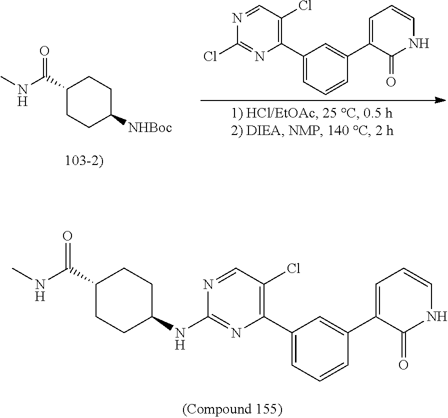 [SMILES: CNC(=O)[C@H]1CC[C@H](NC(=O)OC(C)(C)C)CC1 ; O=c1[nH]cccc1-c1cccc(-c2nc(Cl)ncc2Cl)c1 ; CNC(=O)[C@H]1CC[C@H](Nc2ncc(Cl)c(-c3cccc(-c4ccc[nH]c4=O)c3)n2)CC1]

Synthesis of trans-(1r,4r)-4-((5-chloro-4-(3-(2-oxo-1,2-dihydropyridin-3-yl)phenyl)pyrimidin-2-yl)amino)-N-methylcyclohexane-1-carboxamide (Compound 155). To a solution of trans-tert-butyl ((1r,4r)-4-(methylcarbamoyl)cyclohexyl)carbamate (103-2) (161.14 mg, 628.62 μmol, 1 eq) in HCl/EtOAc (5 mL) was stirred at 25 °C for 0.5 h. The reaction mixture was concentrated under reduced pressure to give a residue and then the residue in NMP (2 mL) was added 3-(3-(2,5-dichloropyrimidin-4-yl)phenyl)pyridin-2(1H)-one (Compound AA) (200 mg, 628.62 μmol, 1 eq) and DIEA (406.22 mg, 3.14 mmol, 547.47 L, 5 eq) at 25 °C, the mixture was stirred at 140 °C for 2 h. LCMS showed 3-(3-(2,5-dichloropyrimidin-4-yl)phenyl)pyridin-2(1H)-one (Compound AA) was consumed completely and desired mass was detected. The residue was purified by prep-HPLC (neutral condition; column: Waters xbridge 150*25 mm 10 um; mobile phase: [water (NH₄HCO₃)-ACN]; gradient: 20%-50% B over 14 min) to give title compound (Compound 155) (20.8 mg, 45.79 μmol, 7.28% yield, 96.41% purity) as a white solid. LCMS (ES+): m/z 438.1 [M+H]⁺.

### Example 104. Synthesis of cis-(1s,4s)-4-((5-chloro-4-(3-(2-oxo-1,2-dihydropyridin-3-yl)phenyl)pyrimidin-2-yl)amino)-N-methylcyclohexane-1-carboxamide (Compound 138)

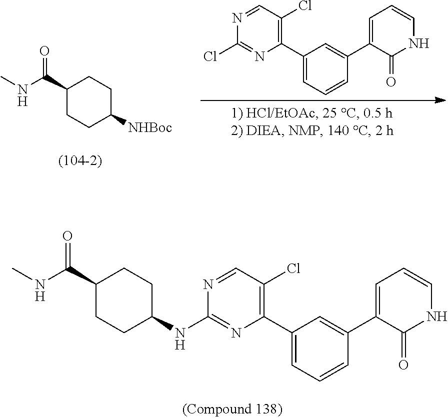 [SMILES: CNC(=O)[C@H]1CC[C@@H](NC(=O)OC(C)(C)C)CC1 ; O=c1[nH]cccc1-c1cccc(-c2nc(Cl)ncc2Cl)c1 ; CNC(=O)[C@H]1CC[C@@H](Nc2ncc(Cl)c(-c3cccc(-c4ccc[nH]c4=O)c3)n2)CC1]

Synthesis of cis-(1s,4s)-4-((5-chloro-4-(3-(2-oxo-1,2-dihydropyridin-3-yl)phenyl)pyrimidin-2-yl)amino)-N-methylcyclohexane-1-carboxamide (Compound 138). A solution of cis-tert-butyl ((1s,4s)-4-(methylcarbamoyl)cyclohexyl)carbamate (104-2) (161.14 mg, 628.62 μmol, 1 eq) in HCl/EtOAc (5 mL) was stirred at 25 °C for 0.5 h. The reaction mixture was concentrated under reduced pressure to give a residue and then the residue in NMP (2 mL) was added 3-(3-(2,5-dichloropyrimidin-4-yl)phenyl)pyridin-2(1H)-one (Compound AA) (0.2 g, 628.62 μmol, 1 eq) and DIEA (406.22 mg, 3.14 mmol, 547.47 μL, 5 eq) at 25 °C and the mixture was stirred at 140 °C for 2 h. LCMS showed 3-(3-(2,5-dichloropyrimidin-4-yl)phenyl)pyridin-2(1H)-one (Compound AA) was consumed completely and desired mass was detected. The reaction did not work up, purified directly. The residue was purified by prep-HPLC (FA condition; column: Welch Ultimate C18 150*25 mm*5 um; mobile phase: [water (FA)-ACN]; gradient: 26%-56% B over 10 min) to give title compound (Compound 138) (36.5 mg, 80.31 μmol, 12.77% yield, 96.35% purity) as a white solid. LCMS (ES+): m/z 438.1 [M+H]⁺.

### Example 105. Synthesis of cis-3-((5-chloro-4-(3-(2-oxo-1,2-dihydropyridin-3-yl)phenyl)pyrimidin-2-yl)amino)-N-methylcyclobutane-1-carboxamide (Compound 156)

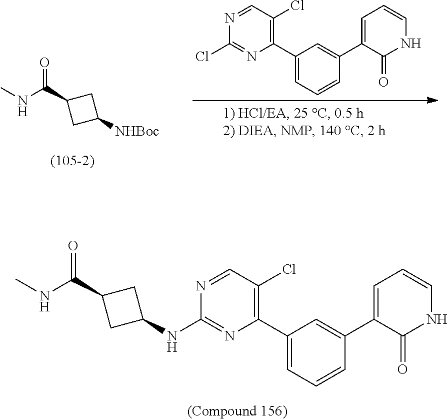 [SMILES: CNC(=O)[C@H]1C[C@@H](NC(=O)OC(C)(C)C)C1 ; O=c1[nH]cccc1-c1cccc(-c2nc(Cl)ncc2Cl)c1 ; CNC(=O)[C@H]1C[C@@H](Nc2ncc(Cl)c(-c3cccc(-c4ccc[nH]c4=O)c3)n2)C1]

Synthesis of cis-3-((5-chloro-4-(3-(2-oxo-1,2-dihydropyridin-3-yl)phenyl)pyrimidin-2-yl)amino)-N-methylcyclobutane-1-carboxamide (Compound 156). A solution of cis-tert-butyl (3-(methylcarbamoyl)cyclobutyl)carbamate (105-2) (143.51 mg, 628.62 μmol, 1 eq) in HCl/EtOAc (5 mL) was stirred at 25 °C for 0.5 h. The reaction mixture was concentrated under reduced pressure to give a residue and then the residue in NMP (2 mL) was added 3-(3-(2,5-dichloropyrimidin-4-yl)phenyl)pyridin-2(1H)-one (Compound AA) (200 mg, 628.62 μmol, 1 eq) and DIEA (406.22 mg, 3.14 mmol, 547.47 μL, 5 eq) at 25 °C. The mixture was stirred at 140 °C for 2 h. LCMS showed 3-(3-(2,5-dichloropyrimidin-4-yl)phenyl)pyridin-2 (1H)-one (Compound AA) was consumed completely and desired mass was detected. The reaction did not work up, purified directly. The residue was purified by prep-HPLC (FA condition; column: Phenomenex luna C18 150*25 mm*10 um; mobile phase: [water(FA)-ACN]; gradient: 24%-54% B over 10 min) to give title compound (Compound 156) (128.7 mg, 308.76 μmol, 49.12% yield, 98.33% purity) as a white solid. LCMS (ES+): m/z 410.2 [M+H]⁺.

### Example 106. Synthesis of trans-(1r,3r)-3-((5-chloro-4-(3-(2-oxo-1,2-dihydropyridin-3-yl)phenyl)pyrimidin-2-yl)amino)-N-methylcyclobutane-1-carboxamide (Compound 139)

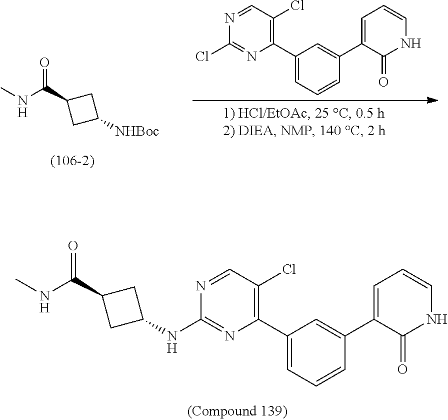 [SMILES: CNC(=O)[C@H]1C[C@H](NC(=O)OC(C)(C)C)C1 ; O=c1[nH]cccc1-c1cccc(-c2nc(Cl)ncc2Cl)c1 ; CNC(=O)[C@H]1C[C@H](Nc2ncc(Cl)c(-c3cccc(-c4ccc[nH]c4=O)c3)n2)C1]

Synthesis of trans-(1r,3r)-3-((5-chloro-4-(3-(2-oxo-1,2-dihydropyridin-3-yl)phenyl)pyrimidin-2-yl)amino)-N-methylcyclobutane-1-carboxamide (Compound 139). A solution of trans-tert-butyl ((1r,3r)-3-(methylcarbamoyl)cyclobutyl)carbamate (106-2) (143.51 mg, 628.62 μmol, 1 eq) in HCl/EtOAc (5 mL) was stirred at 25 °C for 0.5 h. The reaction mixture was concentrated under reduced pressure to give a residue and then the residue in NMP (2 mL) was added 3-(3-(2,5-dichloropyrimidin-4-yl)phenyl)pyridin-2(1H)-one (Compound AA) (0.2 g, 628.62 μmol, 1 eq) and DIEA (406.22 mg, 3.14 mmol, 547.47 μL, 5 eq) at 25 °C. The mixture was stirred at 140 °C for 2 h. LCMS showed 3-(3-(2,5-dichloropyrimidin-4-yl)phenyl)pyridin-2(1H)-one (Compound AA) was consumed completely and desired mass was detected. The reaction did not work up, purified directly. The residue was purified by prep-HPLC (FA condition; column: Welch Ultimate C18 150*25 mm*5 um; mobile phase: [water(FA)-MeOH]; gradient: 20%-50% B over 10 min) to give title compound (Compound 139) (58.7 mg, 137.09 μmol, 21.81% yield, 95.72% purity) as a white solid. LCMS (ES+): m/z 410.0 [M+H]⁺.

### Example 107. Synthesis of cis-3-((5-fluoro-4-(3-(6-oxo-1,6-dihydropyridin-2-yl)phenyl)pyrimidin-2-yl)amino)cyclohexane-1-carboxamide (Compound 112)

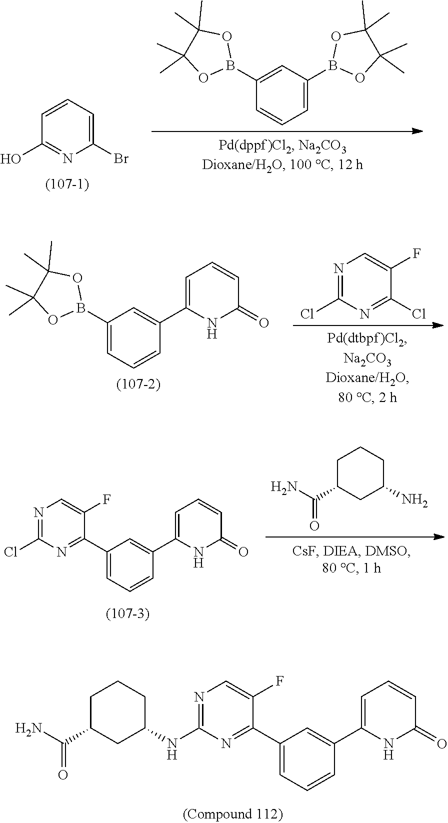

Synthesis of 6-(3-(4,4,5,5-tetramethyl-1,3,2-dioxaborolan-2-yl)phenyl)pyridin-2 (1H)-one (107-2). A mixture of 6-bromopyridin-2-ol (107-1) (200 mg, 1.15 mmol, 1.00 eq), Pd(dppf)Cl₂ (84.1 mg, 114 μmol, 0.100 eq), Na₂CO₃ (365 mg, 3.45 mmol, 3.00 eq) and 4,4,5,5-tetramethyl-2-[3-(4,4,5,5-tetramethyl-1,3,2-dioxaborolan-2-yl)phenyl]-1,3,2-dioxaborolane (455 mg, 1.38 mmol, 1.20 eq) in 1,4-dioxane (2 mL) and H₂O (0.4 mL) was degassed and purged with N₂ at 20 °C for 3 times, and then the mixture was stirred at 100 °C for 12 h under N₂ atmosphere. LC-MS showed 6-bromopyridin-2-ol (107-1) was consumed completely and desired mass was detected. The reaction mixture was quenched by addition H₂O 5 mL, and then diluted with H₂O 5 mL and extracted with EtOAc (10 mL*3). The combined organic layers were washed with sat.aq. NaCl (10 mL), dried over Na₂SO₄, filtered and concentrated under reduced pressure. The residue was purified by prep-HPLC (Phenomenex luna C18 (250*70 mm, 15 um); mobile phase: [H₂O (0.2% FA)-ACN]; gradient: 40%-70% B over 20.0 min) to give the title compound (400 mg, crude) as a white solid. LCMS (ES⁺): m/z 298.1 [M+H]⁺.

Synthesis of 6-(3-(2-chloro-5-fluoropyrimidin-4-yl)phenyl)pyridin-2(1H)-one (107-3). A mixture of 6-(3-(4,4,5,5-tetramethyl-1,3,2-dioxaborolan-2-yl)phenyl)pyridin-2 (1H)-one (107-2) (180.00 mg, 605 μmol, 1.00 eq), 2,4-dichloro-5-fluoro-pyrimidine (101 mg, 605 μmol, 1.00 eq), Na₂CO₃ (192 mg, 1.82 mmol, 3.00 eq), 1,1′-Bis (di-t-butylphosphino)ferrocene palladium dichloride (39.4 mg, 60.5 μmol, 0.100 eq) in 1,4-dioxane (5 mL) and H₂O (1 mL) was degassed and purged with N₂ at 20 °C for 3 times, and then the mixture was stirred at 80 °C for 2 h under N₂ atmosphere. LC-MS showed 6-(3-(4,4,5,5-tetramethyl-1,3,2-dioxaborolan-2-yl)phenyl)pyridin-2(1H)-one (107-2) was consumed completely and the desired mass was detected. The reaction mixture was quenched by addition H₂O 10 mL, and then diluted with H₂O 10 mL and extracted with EtOAc (10 mL*3). The combined organic layers were washed with sat.aq. NaCl (10 mL), dried over Na₂SO₄, filtered and concentrated under reduced pressure. The residue was purified by column chromatography (SiO₂, Petroleum ether:Ethyl acetate=1:0 to 0:1) to give the title compound (107-3) (200 mg, crude) as a brown solid. LCMS (ES⁺): m/z 302.1 [M+H]⁺.

Synthesis of cis-3-((5-fluoro-4-(3-(6-oxo-1,6-dihydropyridin-2-yl)phenyl)pyrimidin-2-yl)amino)cyclohexane-1-carboxamide (Compound 112). To a solution of 6-(3-(2-chloro-5-fluoropyrimidin-4-yl)phenyl)pyridin-2(1H)-one (107-3) (100 mg, 331 μmol, 1.00 eq) in DMSO (3 mL) was added CsF (100 mg, 662 μmol, 24.4 μL, 2.00 eq), cis-3-aminocyclohexane-1-carboxamide (47.1 mg, 331 μmol, 1.00 eq) and DIEA (85.6 mg, 662 mol, 115 μL, 2.00 eq) at 20 °C. The mixture was stirred at 80 °C for 1 h. LC-MS showed -(3-(2-chloro-5-fluoropyrimidin-4-yl)phenyl)pyridin-2(1H)-one (107-3) was consumed completely and the desired mass was detected. The reaction mixture was filtered and the filtrate concentrated under high vacuum. The residue was purified by prep-HPLC (Phenomenex Luna C18 75*30 mm*3 um; mobile phase: [H₂O (0.1% TFA)-ACN]; gradient: 15%-45% B over 8.0 min) to give the title compound (Compound 112) (4.90 mg, 11.2 μmol, 3.39% yield, 93.4% purity) as a white solid. LCMS (ES⁺): m/z 408.2 [M+H]⁺.

### Example 108. Synthesis of trans-N-(4-((5-chloro-4-(1-(cyclopropanecarbonyl) piperidin-3-yl) pyrimidin-2-yl) amino) cyclohexyl)acetamide (Compound 100)

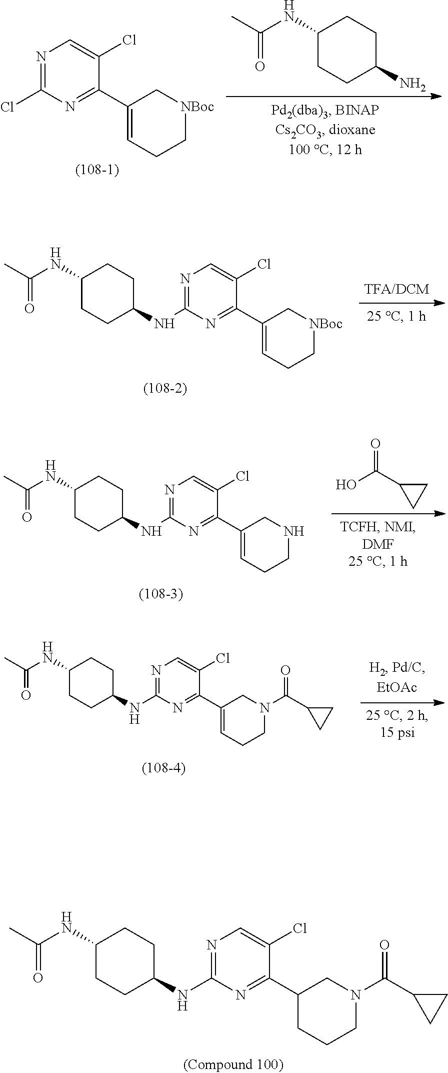

Synthesis of trans-tert-butyl 5-(2-((4-acetamidocyclohexyl) amino)-5-chloropyrimidin-4-yl)-3, 6-dihydropyridine-1 (2H)-carboxylate (108-2). To a solution of tert-butyl 5-(2,5-dichloropyrimidin-4-yl)-3,6-dihydropyridine-1 (2H)-carboxylate (108-1) (423 mg, 1.28 mmol, 1.00 eq) and trans-N-(4-aminocyclohexyl)acetamide (400 mg, 2.56 mmol, 2.00 eq) in dioxane (40 mL) were added Pd₂(dba)₃ (117 mg, 128 μmol, 0.100 eq), BINAP (79.7 mg, 128 μmol, 0.100 eq) and Cs₂CO₃ (1.25 g, 3.84 mmol, 3.00 eq) at 25 °C. The mixture was stirred at 100 °C for 12 h under N₂. LCMS showed tert-butyl 5-(2,5-dichloropyrimidin-4-yl)-3,6-dihydropyridine-1 (2H)-carboxylate (108-1) was consumed completely and the desired mass was detected. The mixture was diluted with H₂O (40 mL) and then extracted with EtOAc (40 mL*3). The combined organic layers were dried over Na₂SO₄, filtered and the filtrate was concentrated under reduced pressure. The mixture was purified by MPLC (SiO₂, PE/EtOAc=1/0 to 0/1) to give the title compound (108-2) (384 mg, 853 μmol, 66.7% yield) as a yellow solid. LCMS (ES⁺): m/z 450.3 [M+H]⁺.

Synthesis of trans-N-(4-((5-chloro-4-(1,2,5,6-tetrahydropyridin-3-yl)pyrimidin-2-yl)amino)cyclohexyl)acetamide (108-3). To a solution of trans-tert-butyl 5-(2-((4-acetamidocyclohexyl)amino)-5-chloropyrimidin-4-yl)-3,6-dihydropyridine-1 (2H)-carboxylate (108-2) (384 mg, 853 μmol, 1.00 eq) in DCM (4.5 mL) was added TFA (1.5 mL) at 25 °C and the mixture was stirred at 25 °C for 1 h. LCMS showed trans-tert-butyl 5-(2-((4-acetamidocyclohexyl)amino)-5-chloropyrimidin-4-yl)-3,6-dihydropyridine-1 (2H)-carboxylate (108-2) was consumed mostly and the desired mass was detected. The mixture was concentrated under reduced pressure to give the title compound (108-3) (400 mg, crude, TFA) as a yellow oil. LCMS (ES⁺): m/z 350.2 [M+H]⁺.

Synthesis of trans-N-(4-((5-chloro-4-(1-(cyclopropanecarbonyl)-1,2,5,6-tetrahydropyridin-3-yl)pyrimidin-2-yl)amino)cyclohexyl)acetamide (108-4). To a solution of cyclopropanecarboxylic acid (74.2 mg, 862 μmol, 68.2 μL, 1.00 eq) in DMF (7.00 mL) were added TCFH (290 mg, 1.03 mmol, 1.20 eq), NMI (12.4 mg, 2.59 mmol, 206 μL, 3.00 eq) and trans-N-(4-((5-chloro-4-(1,2,5,6-tetrahydropyridin-3-yl)pyrimidin-2-yl)amino)cyclohexyl)acetamide (108-3) (400 mg, 862 μmol, 1.00 eq, TFA) at 25 °C, the mixture was stirred at 25 °C for 1 h. LCMS showed trans-N-(4-((5-chloro-4-(1,2,5,6-tetrahydropyridin-3-yl)pyrimidin-2-yl)amino)cyclohexyl)acetamide (108-3) was consumed completely and the desired mass was detected. H₂O (10 mL) was added to the solution, the mixture was extracted with EtOAc (10 mL*3), then the combined organic layers were washed with brine (10 mL*3), dried over Na₂SO₄, filtered and the filtrate was concentrated under reduced pressure to give a residue. The residue was purified by prep-TLC (SiO₂, EtOAc/MeOH=10/1) to give the title compound (108-4) (200 mg, 479 μmol, 41.9% yield) as a white solid. LCMS (ES⁺): m/z 418.2 [M+H]⁺.

Synthesis of trans-N-(4-((5-chloro-4-(1-(cyclopropanecarbonyl)piperidin-3-yl)pyrimidin-2-yl)amino)cyclohexyl)acetamide (Compound 100). To a solution of trans-N-(4-((5-chloro-4-(1-(cyclopropanecarbonyl)-1,2,5,6-tetrahydropyridin-3-yl)pyrimidin-2-yl)amino)cyclohexyl)acetamide (108-4) (200 mg, 457 μmol, 1.00 eq) in EtOAc (4 mL) was added 10% Pd/C (100 mg, 50% purity) at 25 °C, the mixture was stirred at 25 °C under H₂ (15 psi) for 2 h. LCMS showed trans-N-(4-((5-chloro-4-(1-(cyclopropanecarbonyl)-1,2,5,6-tetrahydropyridin-3-yl)pyrimidin-2-yl)amino)cyclohexyl)acetamide (108-4) was consumed mostly and the desired mass was detected. The mixture was filtered, the filtrate was concentrated under reduced pressure. The residue was purified by prep-HPLC (column: Phenomenex Gemini-NX 80*40 mm*3 um; mobile phase: [H₂O (10 mM NH₄HCO₃)-ACN]; gradient: 10%-40% B over 8.0 min) to give the title compound (Compound 100) (17.2 mg, 40.74 μmol, 8.91% yield, 99.8% purity) as a white solid. LCMS (ES⁺): m/z 420.2 [M+H]⁺.

### Example 109. Synthesis of trans-N-(4-((5-chloro-4-(1-(4-fluorobenzoyl)piperidin-3-yl)pyrimidin-2-yl)amino)cyclohexyl)acetamide (Compound 124)

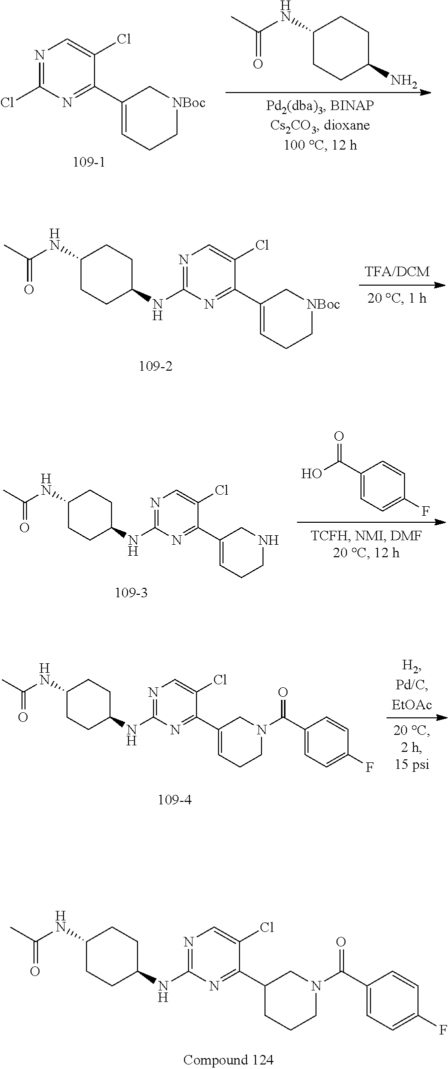

Synthesis of trans-tert-butyl 5-(2-((4-acetamidocyclohexyl)amino)-5-chloropyrimidin-4-yl)-3,6-dihydropyridine-1 (2H)-carboxylate (109-2). To a solution of tert-butyl 5-(2,5-dichloropyrimidin-4-yl)-3,6-dihydropyridine-1 (2H)-carboxylate (109-1) (1.00 g, 3.03 mmol, 1.00 eq) and trans-N-(4-aminocyclohexyl)acetamide (946 mg, 6.06 mmol, 2.00 eq) in dioxane (50 mL) were added Pd₂(dba)₃ (277 mg, 303 μmol, 0.100 eq), BINAP (189 mg, 303 μmol, 0.100 eq) and Cs₂CO₃ (2.96 g, 9.09 mmol, 3.00 eq) at 20 °C. The mixture was stirred at 100 °C for 12 h under N₂. LCMS showed tert-butyl 5-(2,5-dichloropyrimidin-4-yl)-3,6-dihydropyridine-1 (2H)-carboxylate (109-1) was consumed completely and the desired mass was detected. The mixture was diluted with H₂O (20 mL) and then extracted with EtOAc (20 mL*3). The combined organic layers were dried over Na₂SO₄, filtered and the filtrate was concentrated under reduced pressure. The mixture was purified by MPLC (SiO₂, PE/EtOAc=1/0 to 0/1) to give the title compound (109-2) (600 mg, 1.33 mmol, 44.0% yield) as a yellow solid. LCMS (ES+): m/z 450.3 [M+H]⁺.

Synthesis of trans-N-(4-((5-chloro-4-(1,2,5,6-tetrahydropyridin-3-yl)pyrimidin-2-yl)amino)cyclohexyl)acetamide (109-3). To a solution of trans-tert-butyl 5-(2-((4-acetamidocyclohexyl)amino)-5-chloropyrimidin-4-yl)-3,6-dihydropyridine-1 (2H)-carboxylate (109-2) (500 mg, 1.11 mmol, 1.00 eq) in DCM (6 mL) was added TFA (2 mL) at 20 °C, the mixture was stirred at 20 °C for 1 h. LCMS showed trans-tert-butyl 5-(2-((4-acetamidocyclohexyl)amino)-5-chloropyrimidin-4-yl)-3,6-dihydropyridine-1 (2H)-carboxylate (109-2) was consumed completely and the desired mass was detected. The mixture was concentrated under reduced pressure to give the title compound (109-3) (500 mg, crude, TFA) as a yellow oil. LCMS (ES+): m/z 350.2 [M+H]⁺.

Synthesis of trans-N-(4-((5-chloro-4-(1-(4-fluorobenzoyl)-1,2,5,6-tetrahydropyridin-3-yl)pyrimidin-2-yl)amino)cyclohexyl)acetamide (109-4). To a solution of 4-fluorobenzoic acid (151 mg, 1.08 mmol, 1.00 eq) in DMF (8 mL) were added TCFH (363 mg, 1.29 mmol, 1.20 eq) and NMI (265 mg, 3.23 mmol, 258 μL, 3.00 eq) at 20 °C, then trans-N-(4-((5-chloro-4-(1,2,5,6-tetrahydropyridin-3-yl)pyrimidin-2-yl)amino)cyclohexyl)acetamide (109-3) (500 mg, 1.08 mmol, 1.00 eq, TFA) was added to the mixture, the mixture was stirred at 20 °C for 12 h. LCMS showed trans-N-(4-((5-chloro-4-(1,2,5,6-tetrahydropyridin-3-yl)pyrimidin-2-yl)amino)cyclohexyl)acetamide (109-3) was consumed completely and the desired mass was detected. H₂O (10 mL) was added to the solution, the mixture was extracted with EtOAc (10 mL*3), then the combined organic layers were washed with brine (15 mL*3), dried over Na₂SO₄, filtered and the filtrate was concentrated under reduced pressure to give a residue. The residue was purified by prep-TLC (SiO₂, EtOAc/MeOH=10/1) to give the title compound (109-4) (450 mg, 953 μmol, 88.5% yield) as a yellow oil. LCMS (ES+): m/z 472.2 [M+H]⁺.

Synthesis of trans-N-(4-((5-chloro-4-(1-(4-fluorobenzoyl)piperidin-3-yl)pyrimidin-2-yl)amino)cyclohexyl)acetamide (Compound 124). To a mixture of trans-N-(4-((5-chloro-4-(1-(4-fluorobenzoyl)-1,2,5,6-tetrahydropyridin-3-yl)pyrimidin-2-yl)amino)cyclohexyl)acetamide (109-5) (350 mg, 742 μmol, 1.00 eq) in EtOAc (6 mL) was added 10% Pd/C (900 mg, 50% purity) at 20 °C under N₂. The suspension was degassed under vacuum and purged with H₂ 4 times. The mixture was stirred under H₂ (15 psi) at 20 °C for 2 h. LCMS showed that the trans-N-(4-((5-chloro-4-(1-(4-fluorobenzoyl)-1,2,5,6-tetrahydropyridin-3-yl)pyrimidin-2-yl)amino)cyclohexyl)acetamide (109-5) was consumed and the desired mass was detected. The mixture was filtered and the filtrate was concentrated under reduced pressure. This mixture was purified by prep-HPLC (column: Waters Xbridge BEH C18 100*30 mm*10 um; mobile phase: [H₂O (10 mM NH₄HCO₃)-ACN]; gradient: 40%-75% B over 8.0 min) to give the title compound (Compound 124) (28.8 mg, 60.6 μmol, 8.18% yield, 99.8% purity) as white solid. LCMS (ES+): m/z 474.1 [M+H]+.

### Example 110. Synthesis of 1-(4-((5-chloro-4-(1-(4-fluorobenzoyl)piperidin-3-yl)pyrimidin-2-yl)amino)piperidin-1-yl)ethan-1-one (Compound 125)

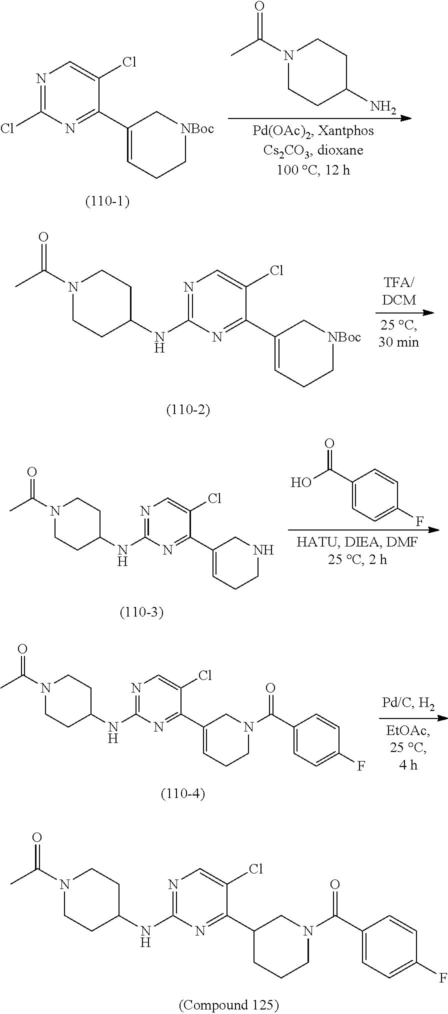

Synthesis of tert-butyl 5-(2-((1-acetylpiperidin-4-yl)amino)-5-chloropyrimidin-4-yl)-3,6-dihydropyridine-1 (2H)-carboxylate (110-2). To a solution of tert-butyl 5-(2,5-dichloropyrimidin-4-yl)-3,6-dihydropyridine-1 (2H)-carboxylate (110-1) (1.00 g, 3.03 mmol, 1.00 eq) in dioxane (10 mL) were added 1-(4-amino-1-piperidyl)ethanone (431 mg, 3.03 mmol, 1.00 eq), Cs₂CO₃ (1.97 g, 6.06 mmol, 2.00 eq), Xantphos (52.6 mg, 90.9 μmol, 0.0300 eq) and Pd(OAc)₂ (34.0 mg, 151 μmol, 0.0500 eq) at 25 °C under N₂ atmosphere. The mixture was stirred at 100 °C for 12 h. LCMS showed tert-butyl 5-(2,5-dichloropyrimidin-4-yl)-3,6-dihydropyridine-1 (2H)-carboxylate (110-1) was consumed completely and the desired mass was detected. Water (9 mL) was added to the mixture at 25 °C. Then the reaction mixture was extracted with EtOAc (8 mL*3). The organic layers were dried over anhydrous sodium sulfate, filtered and the filtrate was concentrated under reduced pressure to give a residue. The residue was purified by column chromatography (SiO₂, Ethyl acetate:Methanol=1:0 to 20:1) to give the title compound (110-2) (420 mg, 785 μmol, 25.9% yield, 81.5% purity) as a white solid. LCMS (ES⁺): m/z 436.2 [M+H]⁺.

Synthesis of 1-(4-((5-chloro-4-(1,2,5,6-tetrahydropyridin-3-yl)pyrimidin-2-yl)amino)piperidin-1-yl)ethan-1-one (110-3). To a solution of tert-butyl 5-(2-((1-acetylpiperidin-4-yl)amino)-5-chloropyrimidin-4-yl)-3,6-dihydropyridine-1 (2H)-carboxylate (110-2) (290 mg, 665 μmol, 1.00 eq) in DCM (3 mL) was added TFA (1.54 g, 13.5 mmol, 1.00 mL, 20.2 eq) at 25 °C. The mixture was stirred at 25 °C for 30 min. LCMS showed tert-butyl 5-(2-((1-acetylpiperidin-4-yl)amino)-5-chloropyrimidin-4-yl)-3,6-dihydropyridine-1 (2H)-carboxylate (110-2) was consumed completely and the desired mass was detected. The reaction mixture was concentrated under reduced pressure to remove the solvent to give the title compound (110-3) (290 mg, crude, TFA) as a yellow oil. LCMS (ES⁺): m/z 336.2 [M+H]⁺.

Synthesis of 1-(4-((5-chloro-4-(1-(4-fluorobenzoyl)-1,2,5,6-tetrahydropyridin-3-yl)pyrimidin-2-yl)amino)piperidin-1-yl)ethan-1-one (110-4). To a solution of 1-(4-((5-chloro-4-(1,2,5,6-tetrahydropyridin-3-yl)pyrimidin-2-yl)amino)piperidin-1-yl)ethan-1-one (110-3) (290 mg, 645 μmol, 1.00 eq, TFA) and 4-fluorobenzoic acid (90.3 mg, 645 μmol, 1.00 eq) in DMF (6 mL) were added HATU (368 mg, 967 μmol, 1.50 eq) and DIPEA (250 mg, 1.93 mmol, 337 μL, 3.00 eq) at 25 °C. The mixture was stirred at 25 °C for 2 h. LCMS showed 1-(4-((5-chloro-4-(1,2,5,6-tetrahydropyridin-3-yl)pyrimidin-2-yl)amino)piperidin-1-yl)ethan-1-one (110-3) was consumed completely and the desired mass was detected. Water (5 mL) was added to the mixture at 25 °C. Then the reaction mixture was extracted with EtOAc (4 mL*3). The organic layers were dried over anhydrous sodium sulfate, filtered and the filtrate was concentrated under reduced pressure to give a residue. The residue was purified by prep-TLC (SiO₂, Ethyl acetate:Methanol=20:1) to give the title compound (110-4) (190 mg, 269 μmol, 41.8% yield, 64.9% purity) as a white solid. LCMS (ES⁺): m/z 458.4 [M+H]⁺.

Synthesis of 1-(4-((5-chloro-4-(1-(4-fluorobenzoyl)piperidin-3-yl)pyrimidin-2-yl)amino)piperidin-1-yl)ethan-1-one (Compound 125). To a solution of 1-(4-((5-chloro-4-(1-(4-fluorobenzoyl)-1,2,5,6-tetrahydropyridin-3-yl)pyrimidin-2-yl)amino)piperidin-1-yl)ethan-1-one (110-5) (190 mg, 415 μmol, 1.00 eq) in EtOAc (4 mL) was added 10% Pd/C (570 mg, 50.0% purity) at 25 °C. The mixture was stirred at 25 °C for 2 h under H₂ (15 psi). LCMS showed 1-(4-((5-chloro-4-(1-(4-fluorobenzoyl)-1,2,5,6-tetrahydropyridin-3-yl)pyrimidin-2-yl)amino)piperidin-1-yl)ethan-1-one (110-5) was not consumed completely and the desired mass was detected. The mixture was stirred at 25 °C for another 2 h under H₂ (15 psi). LCMS showed 1-(4-((5-chloro-4-(1-(4-fluorobenzoyl)-1,2,5,6-tetrahydropyridin-3-yl)pyrimidin-2-yl)amino)piperidin-1-yl)ethan-1-one (110-5) was consumed completely and the desired mass was detected. The reaction mixture was filtered and the filtrate was concentrated under reduced pressure to give a residue. The residue were purified by prep-HPLC (column: Phenomenex Luna C18 100*30 mm*3 um; mobile phase: [H₂O (0.2% FA)-ACN]; gradient: 25%-55% B over 8.0 min) to give the title compound (Compound 125) (21.9 mg, 46.8 μmol, 11.3% yield, 98.2% purity) as a white solid. LCMS (ES⁺): m/z 460.1 [M+H]⁺.

### Example 111. Synthesis of trans-N-(4-((5-chloro-4-(1-(4-fluorobenzoyl)pyrrolidin-3-yl)pyrimidin-2-yl)amino)cyclohexyl)acetamide (Compound 126)

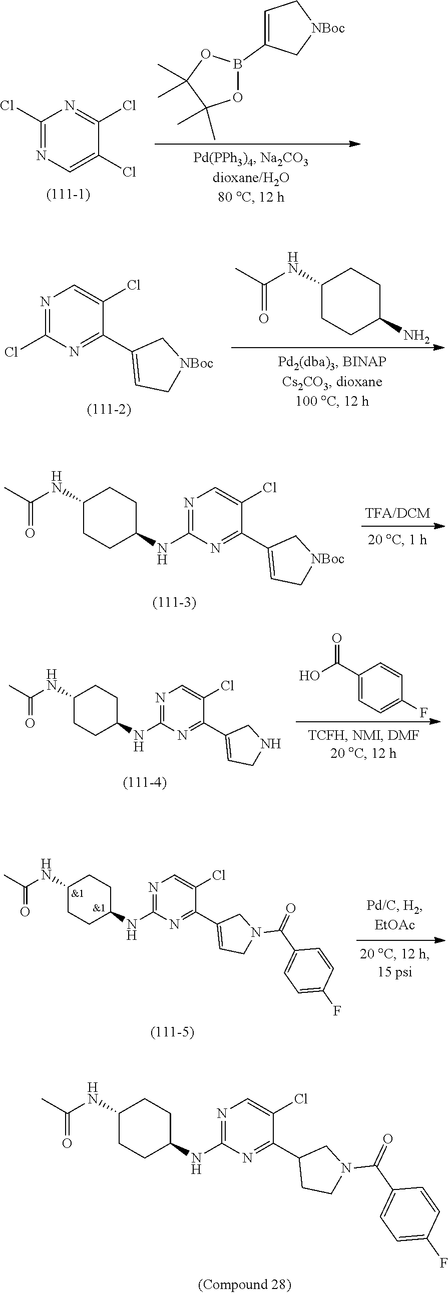

Synthesis of tert-butyl 3-(2,5-dichloropyrimidin-4-yl)-2,5-dihydro-1H-pyrrole-1-carboxylate (111-2). To a solution of 2,4,5-trichloropyrimidine (111-1) (10.0 g, 33.9 mmol, 1.00 eq) and tert-butyl 3-(4,4,5,5-tetramethyl-1,3,2-dioxaborolan-2-yl)-2,5-dihydro-1H-pyrrole-1-carboxylate (7.46 g, 40.7 mmol, 1.20 eq) in dioxane (100 mL) and H₂O (10 mL) were added Na₂CO₃ (7.18 g, 67.8 mmol, 2.00 eq) and Pd(PPh₃)₄ (1.96 g, 1.69 mmol, 0.0500 eq) at 20 °C under N₂ atmosphere. The mixture was stirred at 80 °C for 12 h under N₂ atmosphere. LCMS showed the desired mass was detected. The mixture was diluted with H₂O (350 mL) and then extracted with EtOAc (80 mL*3). The combined organic layers were dried over Na₂SO₄, filtered and the filtrate was concentrated under reduced pressure. The residue was purified by column chromatography (SiO₂, PE/EtOAc=1/0 to 5/1) to give the title compound (111-2) (7.76 g, crude) as a white solid. LCMS (ES+): m/z 260.0 [M+H−56]⁺.

Synthesis of trans-tert-butyl 3-(2-((4-acetamidocyclohexyl)amino)-5-chloropyrimidin-4-yl)-2,5-dihydro-1H-pyrrole-1-carboxylate (111-3). To a solution of tert-butyl 3-(2,5-dichloropyrimidin-4-yl)-2,5-dihydro-1H-pyrrole-1-carboxylate (111-2) (1 g, 3.16 mmol, 1.00 eq) and trans-N-(4-aminocyclohexyl)acetamide (988 mg, 6.33 mmol, 2.00 eq) in dioxane (20 mL) were added Pd₂(dba)₃ (290 mg, 316 μmol, 0.100 eq), BINAP (197 mg, 316 μmol, 0.100 eq) and Cs₂CO₃ (3.09 g, 9.49 mmol, 3.00 eq) at 20 °C. The mixture was stirred at 100 °C for 12 h under N₂. LCMS showed tert-butyl 3-(2,5-dichloropyrimidin-4-yl)-2,5-dihydro-1H-pyrrole-1-carboxylate (111-2) was consumed completely and the desired mass was detected, The mixture was diluted with H₂O (20 mL) and then extracted with EtOAc (20 mL*3). The combined organic layers were dried over Na₂SO₄, filtered and the filtrate was concentrated under reduced pressure. The mixture was purified by MPLC (SiO₂, PE/EtOAC=1/0 to 0/1) to give the title compound (111-3) (600 mg, 1.38 mmol, 43.5% yield) as a yellow oil. LCMS (ES+): m/z 436.3 [M+H]⁺.

Synthesis of trans-N-(4-((5-chloro-4-(2,5-dihydro-1H-pyrrol-3-yl)pyrimidin-2-yl)amino)cyclohexyl)acetamide (111-4). To a solution of trans-tert-butyl 3-(2-((4-acetamidocyclohexyl)amino)-5-chloropyrimidin-4-yl)-2,5-dihydro-1H-pyrrole-1-carboxylate (111-3) (530 mg, 1.22 mmol, 1.00 eq) in DCM (6 mL) was added TFA (2 mL) at 20 °C. The mixture was stirred at 20 °C for 1 h. LCMS shows trans-tert-butyl 3-(2-((4-acetamidocyclohexyl)amino)-5-chloropyrimidin-4-yl)-2,5-dihydro-1H-pyrrole-1-carboxylate (111-3) was consumed and the desired mass was detected. The reaction mixture was concentrated under reduced pressure to give the title compound (111-4) (550 mg, crude, TFA) as a yellow oil. LCMS (ES+): m/z 336.2 [M+H]⁺.

Synthesis of trans-N-(4-((5-chloro-4-(1-(4-fluorobenzoyl)-2,5-dihydro-1H-pyrrol-3-yl)pyrimidin-2-yl)amino)cyclohexyl)acetamide (111-5). To a solution of trans-N-(4-((5-chloro-4-(2,5-dihydro-1H-pyrrol-3-yl)pyrimidin-2-yl)amino)cyclohexyl)acetamide (111-4) (500 mg, 1.11 mmol, 1.00 eq, TFA) and 4-fluorobenzoic acid (187 mg, 1.33 mmol, 1.20 eq) in DMF (3 mL) were added TCFH (374 mg, 1.33 mmol, 1.20 eq) and NMI (274 mg, 3.33 mmol, 266 μL, 3.00 eq) at 20 °C. The mixture was stirred at 20 °C for 12 h. LCMS shows trans-N-(4-((5-chloro-4-(2,5-dihydro-1H-pyrrol-3-yl)pyrimidin-2-yl)amino)cyclohexyl)acetamide (111-4) was consumed and the desired mass was detected. H₂O (10 ml) was added to the mixture and the mixture was extracted with EtOAc (6 mL*3), the combined organic layers were washed with saturated aqueous NaCl solution (30 mL*3), dried over Na₂SO₄, filtered and the filtrate was concentrated under reduced pressure. The residue was purified by column chromatography (SiO₂, DCM/EtOAc=1/0 to 10/1) to give the title compound (111-5) (320 mg, 699 μmol, 62.9% yield) as a yellow oil. LCMS (ES+): m/z 458.2 [M+H]⁺.

Synthesis of trans-N-(4-((5-chloro-4-(1-(4-fluorobenzoyl)pyrrolidin-3-yl)pyrimidin-2-yl)amino)cyclohexyl)acetamide (Compound 126). To a mixture of trans-N-(4-((5-chloro-4-(1-(4-fluorobenzoyl)-2,5-dihydro-1H-pyrrol-3-yl)pyrimidin-2-yl)amino)cyclohexyl)acetamide (111-5) (250 mg, 546 μmol, 1.00 eq) in EtOAc (2 mL) was added 10% Pd/C (250 mg, 50% purity) at 20 °C under N₂. The suspension was degassed under vacuum and purged with H₂ 4 times. The mixture was stirred under H₂ (15 psi) at 20 °C for 12 h. LCMS shows trans-N-(4-((5-chloro-4-(1-(4-fluorobenzoyl)-2,5-dihydro-1H-pyrrol-3-yl)pyrimidin-2-yl)amino)cyclohexyl)acetamide (111-5) was consumed and the desired mass was detected. The mixture was filtered and the filtrate was concentrated under reduced pressure. The residue was purified by prep-HPLC (column: Phenomenex Luna C18 100 * 30 mm*3 um; mobile phase: [H₂O (0.2% FA)-ACN]; gradient: 25%-55% B over 8.0 min) to give the title compound (Compound 126) (14.6 mg, 31.7 μmol, 5.81% yield, 100% purity) as a white solid. LCMS (ES+): m/z 460.1 [M+H]⁺.

### Example 112. Synthesis of cis-3-((5-chloro-4-(1-(4-fluorobenzoyl)pyrrolidin-3-yl)pyrimidin-2-yl)amino)cyclohexane-1-carboxamide (Compound 135)

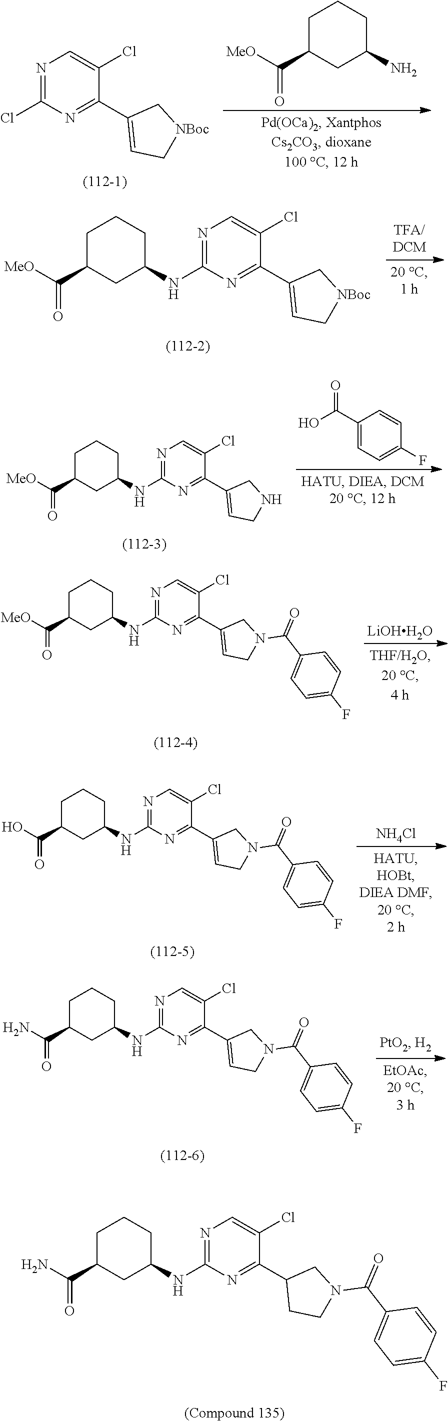

Synthesis of cis-tert-butyl 3-(5-chloro-2-((3-(methoxycarbonyl)cyclohexyl)amino)pyrimidin-4-yl)-2,5-dihydro-J H-pyrrole-1-carboxylate (112-2). To a solution of tert-butyl 3-(2,5-dichloropyrimidin-4-yl)-2,5-dihydro-H-pyrrole-1-carboxylate (112-1) (2.00 g, 6.33 mmol, 1.00 eq) in dioxane (30 mL) were added cis-methyl 3-aminocyclohexane-1-carboxylate (994 mg, 6.33 mmol, 1.00 eq), Cs₂CO₃ (4.12 g, 12.7 mmol, 2.00 eq), Xantphos (110 mg, 190 μmol, 0.0300 eq) and Pd(OAc)₂ (71.1 mg, 316 μmol, 0.0500 eq) at 25 °C under N₂ atmosphere. The mixture was stirred at 100 °C for 12 h. LC-MS showed tert-butyl 3-(2,5-dichloropyrimidin-4-yl)-2,5-dihydro-1H-pyrrole-1-carboxylate (112-1) was consumed completely and the desired mass was detected. The reaction mixture was filtered and the filtrate was concentrated under reduced to give a residue. Water (30 mL) was added to the mixture at 25 °C. The aqueous phase was extracted with EtOAc (20 mL*5). The organic layers were dried over anhydrous sodium sulfate, filtered and the filtrate was concentrated under reduced to give a residue. The residue was purified by column chromatography (SiO₂, Petroleum ether:Ethyl acetate=5:1 to 3:1) to give the title compound (112-2) (626 mg, 1.43 mmol, 22.7% yield) as a yellow oil. LCMS (ES⁺): m/z 437.2 [M+H]⁺.

Synthesis of cis-methyl 3-((5-chloro-4-(2,5-dihydro-1H-pyrrol-3-yl)pyrimidin-2-yl)amino)cyclohexane-1-carboxylate (112-3). To a solution of cis-tert-butyl 3-(5-chloro-2-((3-(methoxycarbonyl)cyclohexyl)amino)pyrimidin-4-yl)-2,5-dihydro-1H-pyrrole-1-carboxylate (112-2) (620 mg, 1.42 mmol, 1.00 eq) in DCM (6 mL) was added TFA (3.02 g, 26.5 mmol, 1.97 mL, 18.7 eq) at 20 °C. The mixture was stirred at 20 °C for 1 h. LC-MS showed cis-tert-butyl 3-(5-chloro-2-((3-(methoxycarbonyl)cyclohexyl)amino)pyrimidin-4-yl)-2,5-dihydro-1H-pyrrole-1-carboxylate (112-2) was consumed completely and the desired mass was detected. The reaction mixture was concentrated under reduced pressure to remove solvent to give the title compound (112-3) (639 mg, crude, TFA) as a yellow oil. LCMS (ES⁺): m/z 337.1 [M+H]⁺.

Synthesis of cis-methyl 3-((5-chloro-4-(1-(4-fluorobenzoyl)-2,5-dihydro-1H-pyrrol-3-yl)pyrimidin-2-yl)amino)cyclohexane-1-carboxylate (112-4). To a solution of 4-fluorobenzoic acid (202 mg, 1.44 mmol, 1.00 eq) in DCM (10 mL) was added HATU (603 mg, 1.59 mmol, 1.10 eq), DIEA (559 mg, 4.33 mmol, 753 μL, 3.00 eq) and cis-methyl 3-((5-chloro-4-(2,5-dihydro-1H-pyrrol-3-yl)pyrimidin-2-yl)amino)cyclohexane-1-carboxylate (112-3) (650 mg, 1.44 mmol, 1.00 eq, TFA) at 20 °C. The mixture was stirred at 20 °C for 1 h. LC-MS showed cis-methyl 3-((5-chloro-4-(2,5-dihydro-1H-pyrrol-3-yl)pyrimidin-2-yl)amino)cyclohexane-1-carboxylate (112-3) was consumed completely and the desired mass was detected. Water (10 mL) was added to the mixture at 20 °C. Then the reaction mixture was extracted with DCM (5 mL*3). The organic layers were dried over anhydrous sodium sulfate, filtered and the filtrate was concentrated under reduced pressure to give a residue. The residue was purified by prep-TLC (SiO₂, Petroleum ether:Ethyl acetate=1:1) to give the title compound (112-4) (580 mg, 1.26 mmol, 87.7% yield) as a white solid. LCMS (ES⁺): m/z 459.2 [M+H]⁺.

Synthesis of cis-3-((5-chloro-4-(1-(4-fluorobenzoyl)-2,5-dihydro-1H-pyrrol-3-yl)pyrimidin-2-yl)amino)cyclohexane-1-carboxylic acid (112-5). To a solution of cis-methyl 3-((5-chloro-4-(1-(4-fluorobenzoyl)-2,5-dihydro-1H-pyrrol-3-yl)pyrimidin-2-yl)amino)cyclohexane-1-carboxylate (112-4) (300 mg, 654 μmol, 1.00 eq) in THF (9 mL) and H₂O (3 mL) was added LiOH·H₂O (165 mg, 3.92 mmol, 6.00 eq) at 20 °C. The mixture was stirred at 20 °C for 3 h. LC-MS showed cis-methyl 3-((5-chloro-4-(1-(4-fluorobenzoyl)-2,5-dihydro-1H-pyrrol-3-yl)pyrimidin-2-yl)amino)cyclohexane-1-carboxylate (112-4) was remaining and the desired mass was detected. Then the mixture was stirred at 20 °C for 1 h. LC-MS showed cis-methyl 3-((5-chloro-4-(1-(4-fluorobenzoyl)-2,5-dihydro-1H-pyrrol-3-yl)pyrimidin-2-yl)amino)cyclohexane-1-carboxylate (112-4) was consumed completely and the desired mass was detected. Water (10 mL) was added to the reaction mixture, then the above mixture was washed with EtOAc (5 mL). HCl (1M) was added to the aqueous phase to adjust pH to 2. Then the mixture was extracted with EtOAc (5 mL*3). The organic layers were dried over anhydrous sodium sulfate, filtered and the filtrate was concentrated under reduced pressure to give the title compound (112-5) (260 mg, crude) as a yellow solid. LCMS (ES⁺): m/z 445.2 [M+H]⁺.

Synthesis of cis-3-((5-chloro-4-(1-(4-fluorobenzoyl)-2,5-dihydro-1H-pyrrol-3-yl)pyrimidin-2-yl)amino)cyclohexane-1-carboxamide (112-6). To a solution of cis-3-((5-chloro-4-(1-(4-fluorobenzoyl)-2,5-dihydro-1H-pyrrol-3-yl)pyrimidin-2-yl)amino)cyclohexane-1-carboxylic acid (112-5) (260 mg, 584 μmol, 1.00 eq) in DMF (5 mL) were added DIEA (227 mg, 1.75 mmol, 305 μL, 3.00 eq), HATU (267 mg, 701 μmol, 1.20 eq), HOBt (39.5 mg, 292 μmol, 0.500 eq) and NH₄Cl (125 mg, 2.34 mmol, 4.00 eq) at 20 °C. The mixture was stirred at 20 °C for 2 h. LC-MS showed cis-3-((5-chloro-4-(1-(4-fluorobenzoyl)-2,5-dihydro-1H-pyrrol-3-yl)pyrimidin-2-yl)amino)cyclohexane-1-carboxylic acid (112-5) was consumed completely and the desired mass was detected. Water (3 mL) was added to the mixture at 20 °C. Then the reaction mixture was extracted with DCM (3 mL*3). The organic layers were dried over anhydrous sodium sulfate, filtered and the filtrate was concentrated under reduced pressure to give a residue. The residue was purified by prep-TLC (SiO₂, Ethyl acetate:Methanol=8:1) to give the title compound (112-6) (200 mg, 451 μmol, 77.1% yield) as a white solid. LCMS (ES⁺): m/z 444.2 [M+H]⁺.

Synthesis of cis-3-((5-chloro-4-(1-(4-fluorobenzoyl)pyrrolidin-3-yl)pyrimidin-2-yl)amino)cyclohexane-1-carboxamide (Compound 135). PtO₂ (200 mg, 881 μmol, 1.95 eq) was added to EtOAc (5 mL) and at 20 °C under N₂ atmosphere, then cis-3-((5-chloro-4-(1-(4-fluorobenzoyl)-2,5-dihydro-1H-pyrrol-3-yl)pyrimidin-2-yl)amino)cyclohexane-1-carboxamide (112-7) (200 mg, 451 μmol, 1.00 eq) was added to the mixture at 20 °C, the suspension was degassed and purged with H₂ for three times. The mixture was stirred at 20 °C for 1 h under H₂ (15 psi) atmosphere. LC-MS showed cis-3-((5-chloro-4-(1-(4-fluorobenzoyl)-2,5-dihydro-1H-pyrrol-3-yl)pyrimidin-2-yl)amino)cyclohexane-1-carboxamide (112-7) was remaining and the desired mass was detected. Then the mixture was stirred at 20 °C for 2 h. LC-MS showed cis-3-((5-chloro-4-(1-(4-fluorobenzoyl)-2,5-dihydro-1H-pyrrol-3-yl)pyrimidin-2-yl)amino)cyclohexane-1-carboxamide (112-7) was consumed completely and the desired mass was detected. The product was filtered and the filtrate was concentrated under reduced pressure to give a residue. The residue was purified by prep-HPLC column: Phenomenex luna C18 100*40 mm*5 um; mobile phase: [H₂O (0.2% FA)-ACN]; gradient: 10%-45% B over 8.0 min) to give the title compound (Compound 135) (4.00 mg, 8.88 μmol, 1.97% yield, 99.0% purity) as a yellow solid. LCMS (ES⁺): m/z 446.1 [M+H]⁺.

### Example 113. Synthesis of 1-(4-((5-chloro-4-(1-(4-fluorobenzoyl)pyrrolidin-3-yl)pyrimidin-2-yl)amino)piperidin-1-yl)ethan-1-one (Compound 127)

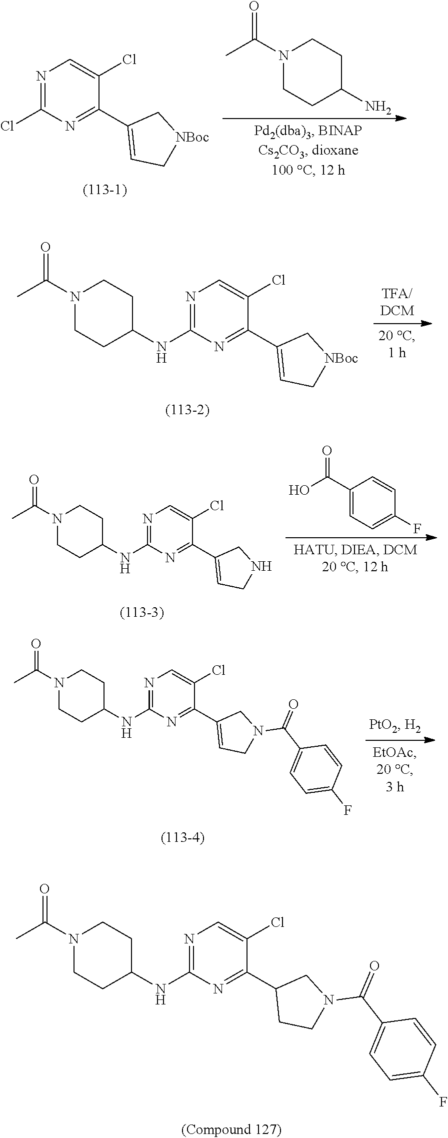

Synthesis of tert-butyl 3-(2-((1-acetylpiperidin-4-yl)amino)-5-chloropyrimidin-4-yl)-2,5-dihydro-1H-pyrrole-J-carboxylate (113-2). To a solution of tert-butyl 3-(2,5-dichloropyrimidin-4-yl)-2,5-dihydropyrrole-1-carboxylate (113-1) (1 g, 3.16 mmol, 1.00 eq) in dioxane (30 mL) were added 1-(4-amino-1-piperidyl)ethanone (674.60 mg, 4.74 mmol, 1.50 eq), Cs₂CO₃ (3.09 g, 9.49 mmol, 3.00 eq), Pd₂(dba)₃ (868.85 mg, 948.82 μmol, 0.3 eq) and BINAP (196.93 mg, 316.27 μmol, 0.1 eq) at 20 °C, the mixture was stirred at 100 °C for 12 h. LCMS showed tert-butyl 3-(2,5-dichloropyrimidin-4-yl)-2,5-dihydropyrrole-1-carboxylate (113-1) was consumed completely and the desired mass was detected. Water (40 mL) was added to the mixture, the reaction was filtered and the filtrate was extracted with EtOAc (40 mL*3), the combined organic layers were concentrated under reduced pressure to give the title compound (113-2) (450 mg, crude) as a brown solid. LCMS (ES+): m/z 422.4 [M+H]⁺.

Synthesis of 1-(4-((5-chloro-4-(2,5-dihydro-1H-pyrrol-3-yl)pyrimidin-2-yl)amino)piperidin-1-yl)ethan-1-one (113-3). A solution of tert-butyl 3-[2-[(1-acetyl-4-piperidyl)amino]-5-chloro-pyrimidin-4-yl]-2,5-dihydropyrrole-1-carboxylate (113-2) (430 mg, 1.02 mmol, 1 eq) in DCM (6 mL) and TFA (2 mL) was stirred at 20 °C for 0.5 h. LCMS showed tert-butyl 3-[2-[(1-acetyl-4-piperidyl)amino]-5-chloro-pyrimidin-4-yl]-2,5-dihydropyrrole-1-carboxylate (113-2) remained and the desired mass was detected. Then the reaction was stirred at 20 °C for another 0.5 h, LCMS showed tert-butyl 3-[2-[(1-acetyl-4-piperidyl)amino]-5-chloro-pyrimidin-4-yl]-2,5-dihydropyrrole-1-carboxylate (113-2) was consumed completely and the desired mass was detected. The reaction was concentrated under reduced pressure to give the title compound (113-3) (440 mg, crude, TFA) as a brown oil. LCMS (ES+): m/z 322.3 [M+H]⁺.

Synthesis of 1-(4-((5-chloro-4-(1-(4-fluorobenzoyl)-2,5-dihydro-1H-pyrrol-3-yl)pyrimidin-2-yl)amino)piperidin-1-yl)ethan-1-one (113-4). To a solution of 1-[4-[[5-chloro-4-(2,5-dihydro-1H-pyrrol-3-yl)pyrimidin-2-yl]amino]-1-piperidyl]ethanone (113-3) (440 mg, 1.01 mmol, 1.00 eq, TFA) and 4-fluorobenzoic acid (141.45 mg, 1.01 mmol, 1.00 eq) in DCM (6 mL) were added HATU (575.80 mg, 1.51 mmol, 1.50 eq) and DIEA (391.44 mg, 3.03 mmol, 527.55 μL, 3.00 eq) at 20 °C, the mixture was stirred at 20 °C for 12 h. LCMS showed 1-[4-[[5-chloro-4-(2,5-dihydro-1H-pyrrol-3-yl)pyrimidin-2-yl]amino]-1-piperidyl]ethanone (113-3) was consumed completely and the desired mass was detected. Water (6 mL) was added to the mixture, the aqueous was extracted with DCM (6 mL*3), then the combined organic layers were dried over Na₂SO₄, filtered and the filtrate was concentrated under reduced pressure. The residue was purified by prep-TLC (SiO₂, EtOAc:MeOH=10:1) to give the title compound (113-4) (100 mg, 225.28 μmol, 22.31% yield) as a yellow oil. LCMS (ES+): m/z 444.3 [M+H]⁺.

Synthesis of 1-(4-((5-chloro-4-(1-(4-fluorobenzoyl)pyrrolidin-3-yl)pyrimidin-2-yl)amino)piperidin-1-yl)ethan-1-one (Compound 127). To a solution of 1-[4-[[5-chloro-4-[1-(4-fluorobenzoyl)-2,5-dihydropyrrol-3-yl]pyrimidin-2-yl]amino]-1-piperidyl]ethanone (113-4) (100 mg, 225.28 μmol, 1 eq) in EtOAc (10 mL) was added PtO₂ (100 mg, 440.38 μmol, 1.95 eq) under N₂ atmosphere. The suspension was degassed and purged with H₂ for three times. The mixture was stirred under H₂ (15 psi) at 20 °C for 1 h. LCMS showed 1-[4-[[5-chloro-4-[1-(4-fluorobenzoyl)-2,5-dihydropyrrol-3-yl]pyrimidin-2-yl]amino]-1-piperidyl]ethanone (113-4) remained and the desired mass was detected. Then the reaction was stirred at 20 °C for another 2 h, LCMS showed 1-[4-[[5-chloro-4-[1-(4-fluorobenzoyl)-2,5-dihydropyrrol-3-yl]pyrimidin-2-yl]amino]-1-piperidyl]ethanone (113-4) was consumed completely and the desired mass was detected. The reaction was filtered and the filtrate was concentrated under reduced pressure. The residue was purified by prep-HPLC (column: Phenomenex Luna C18 100*30 mm*3 um; mobile phase: [H₂O (0.2% FA)-ACN]; gradient: 25%-55% B over 8.0 min) to give the title compound (Compound 127) (12.2 mg, 26.89 μmol, 11.94% yield, 98.29% purity) as a white solid. LCMS (ES+): m/z 446.1 [M+H]⁺.

### Example 114. Synthesis of cis-3-((5-chloro-4-(3-(2-oxopyridin-1 (2H)-yl)phenyl)pyrimidin-2-yl)amino)cyclohexane-1-carboxamide (Compound 136)

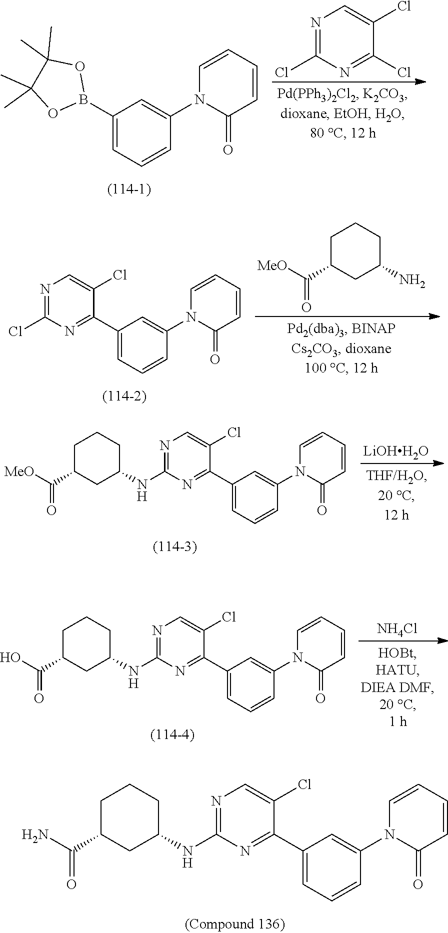

Synthesis of 1-(3-(2,5-dichloropyrimidin-4-yl)phenyl)pyridin-2(1H)-one (114-2). To a solution of 1-(3-(4,4,5,5-tetramethyl-1,3,2-dioxaborolan-2-yl)phenyl)pyridin-2(1H)-one (114-1) (400 mg, 1.35 mmol, 1.00 eq) and 2,4,5-trichloropyrimidine (247 mg, 1.35 mmol, 1.00 eq) in dioxane (10 mL), EtOH (2.5 mL) and H₂O (1 mL) were added Pd(PPh₃)₂Cl₂ (9.45 mg, 13.5 μmol, 0.0100 eq) and K₂CO₃ (930 mg, 6.73 mmol, 5.00 eq) at 20 °C under N₂ atmosphere, the mixture was stirred at 80 °C for 12 h. LCMS showed 1-(3-(4,4,5,5-tetramethyl-1,3,2-dioxaborolan-2-yl)phenyl)pyridin-2(1H)-one (114-1) was consumed completely and the desired mass was detected. Water (10 mL) was added to the mixture and the aqueous was extracted with EtOAc (10 mL*3), the combined organic layers were dried over Na₂SO₄, filtered and the filtrate was concentrated under reduced pressure. The residue was purified by prep-TLC (SiO₂, Petroleum ether:Ethyl acetate=0:1) to give the title compound (114-2) (280 mg, 880 μmol, 65.4% yield) as a white solid. LCMS (ES⁺): m/z 318.2 [M+H]⁺.

Synthesis of cis-methyl 3-((5-chloro-4-(3-(2-oxopyridin-1 (2H)-yl)phenyl)pyrimidin-2-yl)amino)cyclohexane-1-carboxylate (114-3). To a solution of 1-(3-(2,5-dichloropyrimidin-4-yl)phenyl)pyridin-2(1H)-one (114-2) (200 mg, 629 μmol, 1.00 eq) and cis-methyl 3-aminocyclohexane-1-carboxylate (98.8 mg, 629 μmol, 1.00 eq) in dioxane (8 mL) were added Cs₂CO₃ (410 mg, 1.26 mmol, 2.00 eq), BINAP (23.5 mg, 37.7 μmol, 0.0600 eq) and Pd₂(dba)₃ (23.0 mg, 25.1 μmol, 0.0400 eq) at 20 °C under N₂ atmosphere, the mixture was stirred at 100 °C for 12 h. LCMS showed 1-(3-(2,5-dichloropyrimidin-4-yl)phenyl)pyridin-2(1H)-one (114-2) was consumed completely and the desired mass was detected. Water (8 mL) was added to the mixture, then the mixture was extracted with EtOAc (8 mL*3), the combined organic layers were dried over Na₂SO₄, filtered and the filtrate was concentrated under reduced pressure. The residue was purified by prep-TLC (SiO₂, Ethyl acetate:Petroleum ether=1:0) to give the title compound (114-3) (170 mg, 387 μmol, 61.6% yield) as a yellow oil. LCMS (ES⁺): m/z 439.3 [M+H]⁺.

Synthesis of cis-3-((5-chloro-4-(3-(2-oxopyridin-1 (2H)-yl)phenyl)pyrimidin-2-yl)amino)cyclohexane-1-carboxylic acid (114-4). To a solution of cis-methyl 3-((5-chloro-4-(3-(2-oxopyridin-1 (2H)-yl)phenyl)pyrimidin-2-yl)amino)cyclohexane-1-carboxylate (114-3) (170 mg, 387 μmol, 1.00 eq) in THF (2 mL) and H₂O (1 mL) was added LiOH·H₂O (32.5 mg, 775 μmol, 2.00 eq) at 20 °C, the mixture was stirred at 20 °C for 12 h. LCMS showed cis-methyl 3-((5-chloro-4-(3-(2-oxopyridin-1 (2H)-yl)phenyl)pyrimidin-2-yl)amino)cyclohexane-1-carboxylate (114-3) was consumed completely and the desired mass was detected. The reaction was concentrated under reduced pressure to remove THF. Then the aqueous phase was washed with EtOAc (5 mL). HCl (1 M) was added to the aqueous phase to adjust pH to 2-3, then the aqueous was extracted with EtOAc (6 mL*3), the combined organic layers were dried over Na₂SO₄, filtered and the filtrate was concentrated under reduced pressure to give the title compound (114-4) (90.0 mg, crude) as a yellow oil. LCMS (ES⁺): m/z 425.3 [M+H]⁺.

Synthesis of (Compound 136). To a solution of cis-3-((5-chloro-4-(3-(2-oxopyridin-1 (2H)-yl)phenyl)pyrimidin-2-yl)amino)cyclohexane-1-carboxylic acid (114-4) (50.0 mg, 118 μmol, 1.00 eq) in DMF (2 mL) were added NH₄Cl (25.2 mg, 471 μmol, 4.00 eq), DIEA (30.4 mg, 235 μmol, 41.0 μL, 2.00 eq), HOBt (7.95 mg, 58.8 μmol, 0.500 eq) and HATU (53.7 mg, 141 μmol, 1.20 eq) at 20 °C. The mixture was stirred at 20 °C for 1 h. LCMS showed cis-3-((5-chloro-4-(3-(2-oxopyridin-1 (2H)-yl)phenyl)pyrimidin-2-yl)amino)cyclohexane-1-carboxylic acid (114-4) was consumed completely and the desired mass was detected. The organic layers were filtered and the filtrate was concentrated under reduced pressure to give a residue. The residue was purified by prep-HPLC (column: Phenomenex Luna C18 100*30 mm*3 um; mobile phase: [H₂O (0.2% FA)-ACN]; gradient: 15%-55% B over 8.0 min) to give the title compound (Compound 136) (19.1 mg, 45.1 μmol, 38.3% yield, 100% purity) as a pink solid. LCMS (ES⁺): m/z 424.1 [M+H]⁺.

### Example 115. Synthesis of (S)-1-(3-(2-((1-acetylpiperidin-3-yl)amino)-5-chloropyrimidin-4-yl)phenyl)pyridin-2(1H)-one (Compound 129)

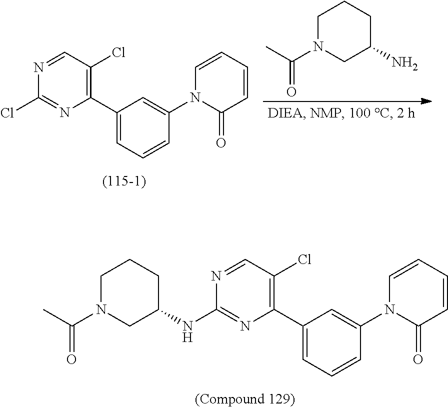

Synthesis of (S)-1-(3-(2-((1-acetylpiperidin-3-yl)amino)-5-chloropyrimidin-4-yl)phenyl)pyridin-2(1H)-one (Compound 129). To a solution of 1-(3-(2,5-dichloropyrimidin-4-yl)phenyl)pyridin-2(1H)-one (300 mg, 942.93 μmol, 1 eq) in NMP (2 mL) was added (S)-1-(3-aminopiperidin-1-yl)ethan-1-one (Compound C) (134.08 mg, 942.93 μmol, 1 eq), DIEA (609.34 mg, 4.71 mmol, 821.21 μL, 5 eq) under N₂, and the reaction mixture was stirred at 100 °C for 2 h under N₂. LCMS showed 1-(3-(2,5-dichloropyrimidin-4-yl)phenyl)pyridin-2 (1H)-one was consumed completely and desired mass was detected. The reaction mixture was filtered and the filtrate was concentrated to give a residue. The residue was purified by reversed-phase HPLC (column: Phenomenex luna C18 150*25 mm*10 um; mobile phase: [water (FA)-ACN]; gradient: 22%-52% B over 10 min) to give title compound (Compound 129) (130 mg, 300.70 μmol, 31.89% yield, 98.05% purity) as a pale yellow solid. LCMS (ES+): m/z 424.0 [M+H]⁺.

### Example 116. Synthesis of cis-N-((1s,4s)-4-((5-chloro-4-(3-(2-oxopyridin-1 (2H)-yl)phenyl)pyrimidin-2-yl)amino)cyclohexyl)acetamide (Compound 93)

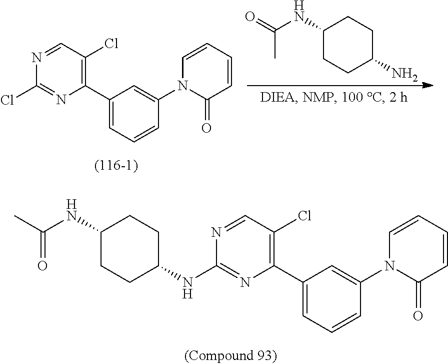 [SMILES: O=c1ccccn1-c1cccc(-c2nc(Cl)ncc2Cl)c1 ; CC(=O)N[C@H]1CC[C@@H](N)CC1 ; CC(=O)N[C@H]1CC[C@@H](Nc2ncc(Cl)c(-c3cccc(-n4ccccc4=O)c3)n2)CC1]

Synthesis of cis-N-((1s,4s)-4-((5-chloro-4-(3-(2-oxopyridin-1 (2H)-yl)phenyl)pyrimidin-2-yl)amino)cyclohexyl)acetamide (Compound 93). To a solution of 1-(3-(2,5-dichloropyrimidin-4-yl)phenyl)pyridin-2(1H)-one (200 mg, 628.62 μmol, 1 eq) in NMP (2 mL) was added cis-N-((1s,4s)-4-aminocyclohexyl)acetamide (Compound D) (98.21 mg, 628.62 μmol, 1 eq), DIEA (406.22 mg, 3.14 mmol, 547.47 μL, 5 eq) under N₂, and the reaction mixture was stirred at 100 °C for 12 h under N₂. LCMS showed 1-(3-(2,5-dichloropyrimidin-4-yl)phenyl)pyridin-2(1H)-one was remained and desired mass was detected. The reaction mixture was filtered and the filtrate was concentrated to give a residue. The residue was purified by reversed-phase HPLC (column: Phenomenex luna C18 150*25 mm*10 um; mobile phase: [water(FA)-ACN]; gradient: 26%-56% B over 10 min) to give title compound (Compound 93) (126.5 mg, 285.52 μmol, 45.42% yield, 98.843% purity) as a pale gray solid. LCMS (ES⁺): m/z 438.0 [M+H]⁺.

### Example 117. Synthesis of trans-N-((1r,4r)-4-((5-chloro-4-(3-(2-oxopyridin-1 (2H)-yl)phenyl)pyrimidin-2-yl)amino)cyclohexyl)acetamide (Compound 102)

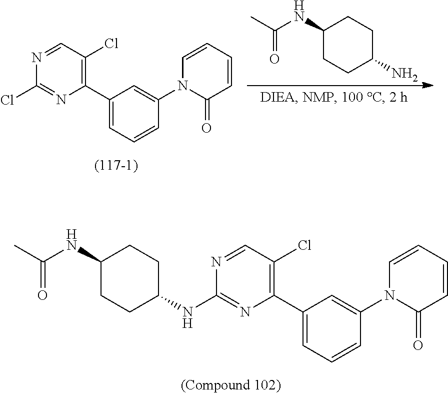 [SMILES: O=c1ccccn1-c1cccc(-c2nc(Cl)ncc2Cl)c1 ; CC(=O)N[C@H]1CC[C@H](N)CC1 ; CC(=O)N[C@H]1CC[C@H](Nc2ncc(Cl)c(-c3cccc(-n4ccccc4=O)c3)n2)CC1]

Synthesis of trans-N-((1r,4r)-4-((5-chloro-4-(3-(2-oxopyridin-1 (2H)-yl)phenyl)pyrimidin-2-yl)amino)cyclohexyl)acetamide (Compound 102). To a solution of 1-(3-(2,5-dichloropyrimidin-4-yl)phenyl)pyridin-2(1H)-one (200 mg, 628.62 μmol, 1 eq) in NMP (2 mL) was added trans-N-((1r,4r)-4-aminocyclohexyl)acetamide (Compound E) (98.21 mg, 628.62 μmol, 1 eq), DIEA (649.96 mg, 5.03 mmol, 875.95 μL, 8 eq) under N₂, and the reaction mixture was stirred at 100 °C for 2 h under N₂. LCMS showed 1-(3-(2,5-dichloropyrimidin-4-yl)phenyl)pyridin-2(1H)-one was consumed completely and desired mass was detected. The reaction mixture was filtered and the filtrate was concentrated to give a residue. The residue was purified by reversed-phase HPLC (column: Phenomenex luna C18 150*25 mm*10 um; mobile phase: [water(FA)-ACN]; gradient: 22%-52% B over 10 min) to give title compound (Compound 102) (94 mg, 211.91 μmol, 33.71% yield, 98.725% purity) as a pale gray solid. LCMS (ES⁺): m/z 438.0 [M+H]⁺.

### Example 118. Synthesis of trans-(1r,4r)-4-((5-chloro-4-(3-(2-oxopyridin-1 (2H)-yl)phenyl)pyrimidin-2-yl)amino)-N-methylcyclohexane-1-carboxamide (Compound 103)

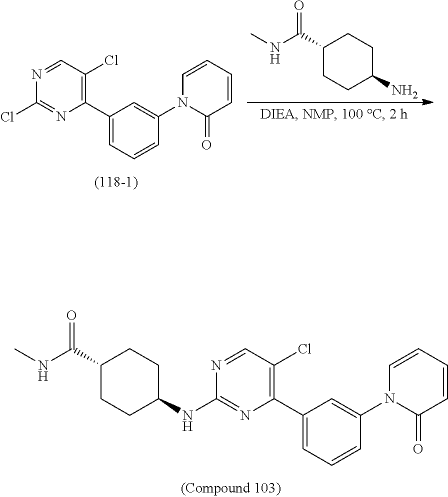 [SMILES: O=c1ccccn1-c1cccc(-c2nc(Cl)ncc2Cl)c1 ; CNC(=O)[C@H]1CC[C@H](N)CC1 ; CNC(=O)[C@H]1CC[C@H](Nc2ncc(Cl)c(-c3cccc(-n4ccccc4=O)c3)n2)CC1]

Synthesis of trans-(1r,4r)-4-((5-chloro-4-(3-(2-oxopyridin-1 (2H)-yl)phenyl)pyrimidin-2-yl)amino)-N-methylcyclohexane-1-carboxamide (Compound 103). To a solution of 1-(3-(2,5-dichloropyrimidin-4-yl)phenyl)pyridin-2(1H)-one (200 mg, 628.62 μmol, 1 eq) in NMP (2 mL) was added trans-(1r,4r)-4-amino-N-methylcyclohexane-1-carboxamide (Compound F) (98.21 mg, 628.62 μmol, 1 eq), DIEA (406.22 mg, 3.14 mmol, 547.47 μL, 5 eq) under N₂, and the reaction mixture was stirred at 100 °C for 2 h under N₂. LCMS showed 1-(3-(2,5-dichloropyrimidin-4-yl)phenyl)pyridin-2(1H)-one was consumed completely and desired mass was detected. The reaction mixture was filtered and the filtrate was concentrated to give a residue. The residue was purified by reversed-phase HPLC (column: Phenomenex luna C18 150*25 mm*10 um; mobile phase: [water(FA)-ACN]; gradient: 22%-52% B over 73 min) to give title compound (Compound 103) (31.4 mg, 69.14 μmol, 11.00% yield, 96.421% purity) as a pale yellow solid. LCMS (ES⁺): m/z 438.0 [M+H]⁺.

### Example 119. Synthesis of cis-(1s,4s)-4-((5-chloro-4-(3-(2-oxopyridin-1 (2H)-yl)phenyl)pyrimidin-2-yl)amino)-N-methylcyclohexane-1-carboxamide (Compound 104)

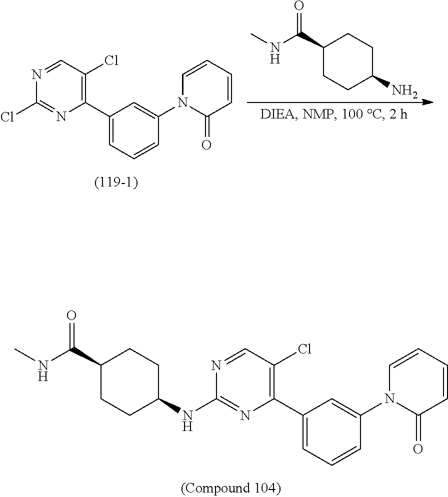 [SMILES: O=c1ccccn1-c1cccc(-c2nc(Cl)ncc2Cl)c1 ; CNC(=O)[C@H]1CC[C@@H](N)CC1 ; CNC(=O)[C@H]1CC[C@@H](Nc2ncc(Cl)c(-c3cccc(-n4ccccc4=O)c3)n2)CC1]

Synthesis of cis-(1s,4s)-4-((5-chloro-4-(3-(2-oxopyridin-1 (2H)-yl)phenyl)pyrimidin-2-yl)amino)-N-methylcyclohexane-1-carboxamide (Compound 104). To a solution of 1-(3-(2,5-dichloropyrimidin-4-yl)phenyl)pyridin-2(1H)-one (200 mg, 628.62 μmol, 1 eq) in NMP (2 mL) was added cis-(1s,4s)-4-amino-N-methylcyclohexane-1-carboxamide (Compound G) (98.21 mg, 628.62 μmol, 1 eq), DIEA (649.96 mg, 5.03 mmol, 875.95 μL, 8 eq) under N₂, and the reaction mixture was stirred at 100 °C for 2 h under N₂. LCMS showed 1-(3-(2,5-dichloropyrimidin-4-yl)phenyl)pyridin-2(1H)-one was consumed completely and desired mass was detected. The reaction mixture was filtered and the filtrate was concentrated to give a residue. The residue was purified by reversed-phase HPLC (column: Phenomenex luna C18 150*25 mm*10 um; mobile phase: [water (FA)-ACN]; gradient: 24%-54% B over 10 min) to give title compound (Compound 104) (113.7 mg, 255.85 μmol, 40.70% yield, 98.540% purity) as a pale yellow solid. LCMS (ES+): m/z 438.0 [M+H]⁺.

### Example 120. Synthesis of cis-3-((5-chloro-4-(3-(2-oxopyridin-1 (2H)-yl)phenyl)pyrimidin-2-yl)amino)-N-methylcyclobutane-1-carboxamide (Compound 94)

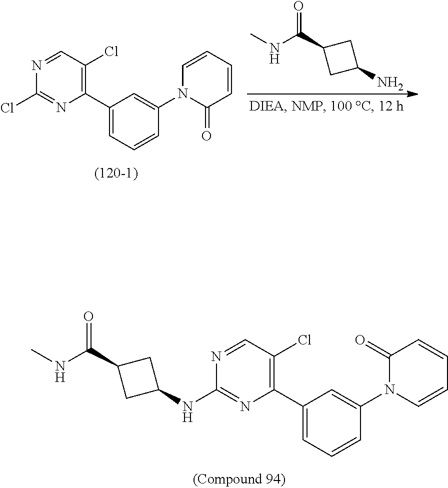

Synthesis of cis-3-((5-chloro-4-(3-(2-oxopyridin-1 (2H)-yl)phenyl)pyrimidin-2-yl)amino)-N-methylcyclobutane-1-carboxamide (Compound 94). To a solution of 1-(3-(2,5-dichloropyrimidin-4-yl)phenyl)pyridin-2(1H)-one (200 mg, 628.62 μmol, 1 eq) in NMP (2 mL) was added cis-3-amino-N-methylcyclobutane-1-carboxamide (Compound H) (80.57 mg, 628.62 μmol, 1 eq), DIEA (812.45 mg, 6.29 mmol, 1.09 mL, 10 eq) under N₂, and the reaction mixture was stirred at 100 °C for 12 h under N₂. LCMS showed 1-(3-(2,5-dichloropyrimidin-4-yl)phenyl)pyridin-2(1H)-one was consumed completely and desired mass was detected. The reaction mixture was filtered and the filtrate was concentrated to give a residue. The residue was purified by reversed-phase HPLC (column: Phenomenex luna C18 150*25 mm*10 um; mobile phase: [water (FA)-ACN]; gradient: 18%-48% B over 10 min) to give title compound (Compound 94) (135.5 mg, 329.79 μmol, 52.46% yield, 99.756% purity) as a white solid. LCMS (ES+): m/z 410.0 [M+H]⁺.

### Example 121. Synthesis of cis-methyl 3-((5-chloro-4-(3-(4-fluoro-2-oxopyridin-1 (2H)-yl)phenyl)pyrimidin-2-yl)amino)cyclohexane-1-carboxylate (Compound 168)

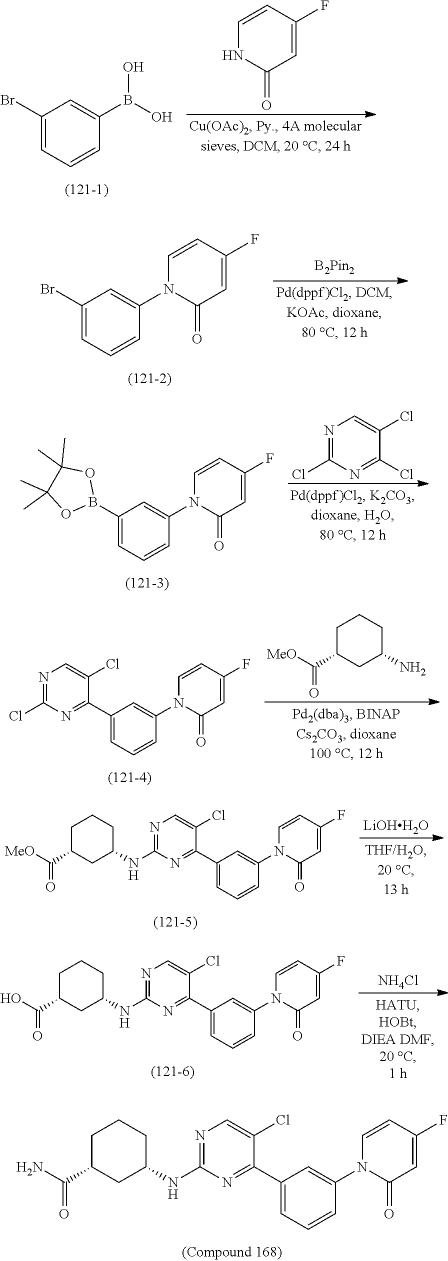

Synthesis of 1-(3-bromophenyl)-4-fluoropyridin-2(1H)-one (121-2). To a solution of (3-bromophenyl)boronic acid (121-1) (7.10 g, 35.4 mmol, 2.00 eq) and 4-fluoropyridin-2 (1H)-one (2.00 g, 17.7 mmol, 1.00 eq) in DCM (100 mL) were added Cu(OAc)₂ (161 mg, 884 μmol, 0.0500 eq), Py (1.40 g, 17.7 mmol, 1.43 mL, 1.00 eq) and 4 A molecular sieves (400 mg) at 20 °C. The mixture was stirred at 20 °C for 12 h. LCMS showed (3-bromophenyl)boronic acid (121-1) was remained, desired target MS was detected. Then (3-bromophenyl)boronic acid (121-1) (7.10 g, 35.4 mmol, 2.00 eq), Cu(OAc)₂ (161 mg, 884 mol, 0.0500 eq), Py (1.40 g, 17.7 mmol, 1.43 mL, 1.00 eq) and 4 A molecular sieves (400 mg) were added to the mixture, the mixture was stirred at 20 °C for another 12 h, LCMS showed (3-bromophenyl)boronic acid (121-1) was consumed, desired target MS was detected. H₂O (120 mL) was added to the solution, the mixture was extracted with DCM (120 mL*3), then the combined organic lays were dried over Na₂SO₄, filtered and the filtrate was concentrated in vacuo to give the crude product. The crude product was purified by MPLC (SiO₂, Petroleum ether:Ethyl acetate=1:0 to 0:1) to give the title compound (121-2) (4.50 g, 16.8 mmol, 94.92% yield) as a white solid. LCMS (ES+): m/z 268.0 [M+H]⁺.

Synthesis of 4-fluoro-1-(3-(4,4,5,5-tetramethyl-1,3,2-dioxaborolan-2-yl)phenyl)pyridin-2(1H)-one (121-3). To a solution of 1-(3-bromophenyl)-4-fluoropyridin-2 (1H)-one (121-2) (2.00 g, 7.46 mmol, 1.00 eq) and 4,4,5,5-tetramethyl-2-(4,4,5,5-tetramethyl-1,3,2-dioxaborolan-2-yl)-1,3,2-dioxaborolane (2.46 g, 9.70 mmol, 1.30 eq) in dioxane (40 mL) were added KOAc (2.93 g, 29.8 mmol, 4.00 eq) and Pd(dppf)Cl₂·CH₂Cl₂ (609 mg, 746 μmol, 0.100 eq) at 20 °C. The mixture was stirred at 80 °C for 12 h under N₂. LCMS showed 1-(3-bromophenyl)-4-fluoropyridin-2(1H)-one (121-2) was consumed, and the desired target MS was detected. H₂O (45 mL) was added to the solution, the mixture was extracted with EtOAc (45 mL*3), then the combined organic layers were dried over Na₂SO₄, filtered and the filtrate was concentrated in vacuo to give the crude product. The crude product was purified by MPLC (SiO₂, Petroleum ether:Ethyl acetate=1:0 to 0:1) to give the title compound (121-3) (800 mg, 2.54 mmol, 34.03% yield) as a yellow oil. LCMS (ES⁺): m/z 316.0 [M+H]⁺

Synthesis of 1-(3-(2,5-dichloropyrimidin-4-yl)phenyl)-4-fluoropyridin-2(1H)-one (121-4). To a stirred solution of 4-fluoro-1-(3-(4,4,5,5-tetramethyl-1,3,2-dioxaborolan-2-yl)phenyl)pyridin-2(1H)-one (121-3) (700 mg, 2.22 mmol, 1.00 eq) and 2,4,5-trichloropyrimidine (407 mg, 2.22 mmol, 1.00 eq) in dioxane (5 mL) and H₂O (1 mL) were added Pd(dppf)Cl₂ (163 mg, 222 μmol, 0.100 eq) and K₂CO₃ (921 mg, 6.66 mmol, 3.00 eq) at 20 °C under N₂ atmosphere. The mixture was stirred at 80 °C for 12 h. LCMS showed 4-fluoro-1-(3-(4,4,5,5-tetramethyl-1,3,2-dioxaborolan-2-yl)phenyl)pyridin-2(1H)-one (121-3) was consumed, and the desired target MS was detected. H₂O (8 mL) was added to the solution, the mixture was extracted with EtOAc (8 mL*3), then the combined organic lays were dried over Na₂SO₄, filtered and the filtrate was concentrated in vacuo to give the crude product. The crude product was purified by prep-TLC (SiO₂, PE:EtOAc=1:1) to give the title compound (121-4) (610 mg, 1.81 mmol, 81.70% yield) as a yellow oil. LCMS (ES⁺): m/z 336.0 [M+H]⁺

Synthesis of rac-methyl (1R,3S)-3-((5-chloro-4-(3-(4-fluoro-2-oxopyridin-1 (2H)-yl)phenyl)pyrimidin-2-yl)amino)cyclohexane-1-carboxylate (121-5). To a solution of 1-(3-(2,5-dichloropyrimidin-4-yl)phenyl)-4-fluoropyridin-2(1H)-one (121-4) (610 mg, 1.81 mmol, 1.00 eq) and rac-methyl (1R,3S)-3-aminocyclohexane-1-carboxylate (351 mg, 1.81 mmol, 1.00 eq, HCl) in dioxane (8 mL) were added Cs₂CO₃ (1.77 g, 5.44 mmol, 3.00 eq), BINAP (79.1 mg, 127 umol, 0.0700 eq) and Pd₂(dba)₃ (83.1 mg, 90.7 μmol, 0.0500 eq) at 20 °C, the mixture was stirred at 100 °C for 12 h under N₂ atmosphere. LCMS showed 1-(3-(2,5-dichloropyrimidin-4-yl)phenyl)-4-fluoropyridin-2(1H)-one (121-4) was consumed, and the desired target MS was detected. H₂O (10 mL) was added to the solution, the mixture was extracted with EtOAc (10 mL*3), then the combined organic lays were dried over Na₂SO₄, filtered and the filtrate was concentrated in vacuo to give the crude product. The mixture was purified by prep-TLC (SiO₂, PE:EtOAc=1:1) to give the title compound (121-5) (230 mg, 503 μmol, 27.74% yield) as a yellow solid. LCMS (ES⁺): m/z 457.2 [M+H]⁺

Synthesis of rac-(1R,3S)-3-((5-chloro-4-(3-(4-fluoro-2-oxopyridin-1 (2H)-yl)phenyl)pyrimidin-2-yl)amino)cyclohexane-1-carboxylic acid (121-6). To a solution of rac-methyl (1R,3S)-3-((5-chloro-4-(3-(4-fluoro-2-oxopyridin-1 (2H)-yl)phenyl)pyrimidin-2-yl)amino)cyclohexane-1-carboxylate (121-5) (180 mg, 394 μmol, 1.00 eq) in THF (3 mL) and H₂O (1.5 mL) was added LiOH·H₂O (33.1 mg, 788 μmol, 2.00 eq) at 20 °C, the mixture was stirred at 20 °C for 12 h. LCMS showed rac-methyl (1R,3S)-3-((5-chloro-4-(3-(4-fluoro-2-oxopyridin-1 (2H)-yl)phenyl)pyrimidin-2-yl)amino)cyclohexane-1-carboxylate (121-5) was remaining, and the desired target MS was detected. Then the mixture was stirred at 20 °C for another 1 h, LCMS showed rac-methyl (1R,3S)-3-((5-chloro-4-(3-(4-fluoro-2-oxopyridin-1 (2H)-yl)phenyl)pyrimidin-2-yl)amino)cyclohexane-1-carboxylate (121-5) was consumed mostly, and the desired target MS was detected. The mixture was concentrated in vacuo to remove THF. The aqueous was washed with EtOAc (3 mL), then HCl (1 M) was added to the aqueous to adjust pH=4 and then it was extracted with EtOAc (5 mL*3). The combined organic layers were dried over Na₂SO₄, filtered and the filtrate was concentrated in vacuo to give the title compound (121-6) (130 mg, crude) as a yellow oil. LCMS (ES⁺): m/z 443.1 [M+H]⁺

Synthesis of rac-(1R,3S)-3-((5-chloro-4-(3-(4-fluoro-2-oxopyridin-1 (2H)-yl)phenyl)pyrimidin-2-yl)amino)cyclohexane-1-carboxamide (Compound 168). To a solution of rac-(1R,3S)-3-((5-chloro-4-(3-(4-fluoro-2-oxopyridin-1 (2H)-yl)phenyl)pyrimidin-2-yl)amino)cyclohexane-1-carboxylic acid (121-6) (130 mg, 294 μmol, 1.00 eq) and NH₄Cl (62.8 mg, 1.17 mmol, 4.00 eq) in DMF (1 mL) was added HATU (134 mg, 352 μmol, 1.20 eq), HOBt (19.8 mg, 147 μmol, 0.500 eq) and DIEA (190 mg, 1.47 mmol, 256 μL, 5.00 eq) at 20 °C, then the mixture was stirred at 20 °C for 1 h. LCMS showed rac-(1R,3S)-3-((5-chloro-4-(3-(4-fluoro-2-oxopyridin-1 (2H)-yl)phenyl)pyrimidin-2-yl)amino)cyclohexane-1-carboxylic acid was consumed, and the desired target MS was detected. The mixture was concentrated in vacuo. The residue was purified by prep-HPLC (column: Phenomenex Luna C18 75*30 mm*3 um; mobile phase: [H₂O (0.2% FA)-ACN]; gradient: 20%-50% B over 8.0 min) to give the title compound (Compound 168) (40.4 mg, 90.8 μmol, 30.9% yield, 99.3% purity) as a white solid. LCMS (ES⁺): m/z 442.1 [M+H]⁺.

### Example 122. Synthesis of cis-3-((5-fluoro-4-(3-(oxetan-3-yl)phenyl)pyrimidin-2-yl)amino)cyclohexane-1-carboxamide (Compound 170)

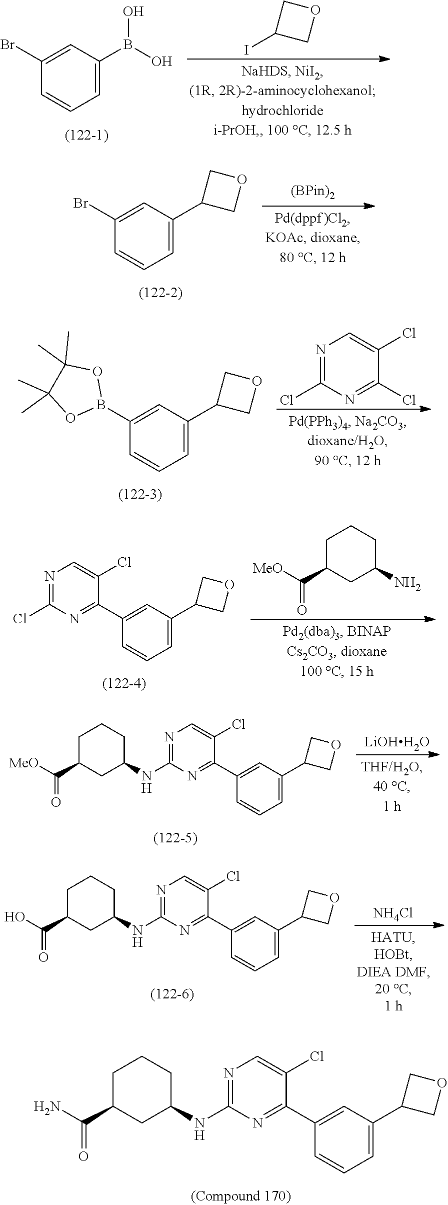

Synthesis of 3-(3-bromophenyl)oxetane (122-2). To a solution of (3-bromophenyl)boronic acid (122-1) (2.00 g, 9.96 mmol, 1.00 eq) in i-PrOH (40 mL) were added NaHMDS (1.00 M, 14.9 mL, 1.50 eq), NiI₂ (311 mg, 996 μmol, 53.4 μL, 0.100 eq) and (1R,2R)-2-aminocyclohexanol; hydrochloride (151 mg, 996 umol, 0.100 eq) at 20 °C under N₂ atmosphere. The mixture was stirred at 20 °C for 30 min. Then 3-iodooxetane (1.83 g, 9.96 mmol, 1.00 eq) was added to the mixture at 20 °C. The mixture was stirred at 100 °C for 12 h. TLC (SiO₂, Petroleum ether:Ethyl acetate=5:1) indicated two new spots formed. Water (20 mL) was added to the mixture at 20 °C. Then the reaction mixture was extracted with EtOAc (15 mL*3). The organic layers were dried over anhydrous sodium sulfate, filtered and the filtrate was concentrated under reduced pressure to give a residue. The residue was purified by MPLC (SiO₂, PE/EtOAc=1/0 to 5/1) to give the title compound (122-2) (1.30 g, 6.10 mmol, 61.3% yield) as a white solid. The product couldn't detected by LCMS. It was confirmed by ¹H NMR (400 MHz, CHLOROFORM-d) 6=7.58 (s, 1H), 7.44 (d, J=7.8 Hz, 1H), 7.36 (d, J=7.7 Hz, 1H), 7.30-7.25 (m, 1H), 5.10 (dd, J=6.2, 8.3 Hz, 2H), 4.77 (t, J=6.4 Hz, 2H), 4.27-4.16 (m, 1H)

Synthesis of 4,4,5,5-tetramethyl-2-(3-(oxetan-3-yl)phenyl)-1,3,2-dioxaborolane (122-3). To a solution of 3-(3-bromophenyl)oxetane (122-2) (700 mg, 3.29 mmol, 1.00 eq) in dioxane (5 mL) were added 4,4,5,5-tetramethyl-2-(4,4,5,5-tetramethyl-1,3,2-dioxaborolan-2-yl)-1,3,2-dioxaborolane (1.00 g, 3.94 mmol, 1.20 eq), KOAc (645 mg, 6.57 mmol, 2.00 eq) and Pd(dppf)Cl₂ (120 mg, 164 μmol, 0.0500 eq) at 20 °C under N₂. The mixture was stirred at 80 °C for 12 h. TLC (SiO₂, Petroleum ether:Ethyl acetate=3:1) indicated 3-(3-bromophenyl)oxetane was consumed completely and two new spots formed. To the mixture was added H₂O (10 mL) and the mixture was extracted with EtOAc (10 mL*3). The combined organic layers were washed with brine (10 mL*3), dried over Na₂SO₄, filtered and the filtrate was concentrated under reduced pressure. The residue was purified by prep-TLC (SiO₂, PE/EtOAc=3/1) to give the title compound (122-3) (580 mg, 2.23 mmol, 67.9% yield) as a white solid. The product couldn't detected by LCMS. It was confirmed by ¹H NMR (400 MHz, DMSO-d₆) δ=7.69 (s, 1H), 7.60-7.52 (m, 2H), 7.42-7.37 (m, 1H), 4.99-4.92 (m, 2H), 4.62-4.57 (m, 2H), 4.31-4.22 (m, 1H), 1.30 (s, 12H)

Synthesis of 2-chloro-5-fluoro-4-(3-(oxetan-3-yl)phenyl)pyrimidine (122-4). To a solution of 4,4,5,5-tetramethyl-2-(3-(oxetan-3-yl)phenyl)-1,3,2-dioxaborolane (122-3) (580 mg, 2.23 mmol, 1.00 eq) in dioxane (10 mL) and H₂O (1 mL) were added 2,4-dichloro-5-fluoropyrimidine (447 mg, 2.68 mmol, 1.20 eq), Na₂CO₃ (473 mg, 4.46 mmol, 2.00 eq) and Pd(PPh₃)₄ (51.5 mg, 44.6 μmol, 0.0200 eq) at 20 °C under N₂ atmosphere. The mixture was stirred at 90 °C for 12 h. LCMS showed the desired mass was detected. To the mixture was added H₂O (10 mL) and the mixture was extracted with EtOAc (10 mL*3). The combined organic layers were washed with brine (10 mL*3), dried over Na₂SO₄, filtered and the filtrate was concentrated under reduced pressure. The mixture was purified by prep-TLC (SiO₂, PE/EtOAc=1/1) to give the title compound (122-4) (450 mg, 1.70 mmol, 76.3% yield) as a white solid. LCMS (ES⁺): m/z 265.0 [M+H]⁺.

Synthesis of cis-methyl 3-((5-fluoro-4-(3-(oxetan-3-yl)phenyl)pyrimidin-2-yl)amino)cyclohexane-1-carboxylate (122-5). To a solution of 2-chloro-5-fluoro-4-(3-(oxetan-3-yl)phenyl)pyrimidine (122-4) (230 mg, 869 μmol, 1.00 eq) and cis-methyl 3-aminocyclohexane-1-carboxylate (168 mg, 869 μmol, 1.00 eq, HCl) in dioxane (3 mL) were added Cs₂CO₃ (566 mg, 1.74 mmol, 2.00 eq), BINAP (32.5 mg, 52.1 μmol, 0.0600 eq) and Pd₂(dba)₃ (31.8 mg, 34.8 μmol, 0.0400 eq) at 20 °C. The resulting mixture was stirred at 100 °C for 12 h under N₂. LCMS showed 2-chloro-5-fluoro-4-(3-(oxetan-3-yl)phenyl)pyrimidine (122-4) remained and the desired mass was detected. Then cis-methyl 3-aminocyclohexane-1-carboxylate (168 mg, 1.00 eq), Cs₂CO₃ (566 mg, 2.00 eq), BINAP (32.5 mg, 0.0600 eq) and Pd₂(dba)₃ (31.8 mg, 0.0400 eq) were added to the mixture at 20 °C. The mixture was stirred at 100 °C for 3 h under N₂ atmosphere. LCMS showed 2-chloro-5-fluoro-4-(3-(oxetan-3-yl)phenyl)pyrimidine (122-4) was consumed completely and the desired mass was detected. To the mixture was added H₂O (10 mL) and the mixture was extracted with EtOAc (10 mL*3). The combined organic layers were washed with brine (10 mL*3), dried over Na₂SO₄, filtered and the filtrate was concentrated under reduced pressure. The mixture was purified by prep-TLC (SiO₂, PE/EtOAc=3/1) to give the title compound (122-5) (230 mg, 597 μmol, 68.7% yield) as a white solid. LCMS (ES⁺): m/z 386.2 [M+H]⁺.

Synthesis of cis-3-((5-fluoro-4-(3-(oxetan-3-yl)phenyl)pyrimidin-2-yl)amino)cyclohexane-1-carboxylic acid (122-6). To a solution of cis-methyl 3-((5-fluoro-4-(3-(oxetan-3-yl)phenyl)pyrimidin-2-yl)amino)cyclohexane-1-carboxylate (122-5) (210 mg, 545 μmol, 1.00 eq) in THE (2 mL) and H₂O (1 mL) was added LiOH·H₂O (45.7 mg, 1.09 mmol, 2.00 eq) at 20 °C, the mixture was stirred at 40 °C for 1 h. LCMS showed cis-methyl 3-((5-fluoro-4-(3-(oxetan-3-yl)phenyl)pyrimidin-2-yl)amino)cyclohexane-1-carboxylate (122-5) was consumed and the desired mass was detected. The mixture was concentrated under reduced pressure to remove THF. Then the remaining aqueous phase was washed with EtOAc (5 mL). Then the aqueous layer was acidified by HCl (1 M) to adjust pH=3 and it was extracted with EtOAc (5 mL*3). The combined organic layers were dried over Na₂SO₄ and then concentrated under reduced pressure to give the title compound (122-6) (160 mg, crude) as colorless oil. LCMS (ES⁺): m/z 372.2 [M+H]⁺.

Synthesis of cis-3-((5-fluoro-4-(3-(oxetan-3-yl)phenyl)pyrimidin-2-yl)amino)cyclohexane-1-carboxamide (Compound 170). To a solution of cis-3-((5-fluoro-4-(3-(oxetan-3-yl)phenyl)pyrimidin-2-yl)amino)cyclohexane-1-carboxylic acid (122-6) (130 mg, 350 μmol, 1.00 eq) in DMF (1 mL) were added HATU (160 mg, 420 μmol, 1.20 eq), HOBt (23.7 mg, 175 μmol, 0.500 eq), DIEA (90.5 mg, 700 μmol, 122 μL, 2.00 eq) and NH₄Cl (74.9 mg, 1.40 mmol, 4.00 eq) at 20 °C, the mixture was stirred at 20 °C for 1 h. LCMS showed cis-3-((5-fluoro-4-(3-(oxetan-3-yl)phenyl)pyrimidin-2-yl)amino)cyclohexane-1-carboxylic acid (122-6) was consumed and the desired mass was detected. To the mixture was added H₂O (5 mL) and the mixture was extracted with EtOAc (5 mL*3). The combined organic layers were washed with brine (5 mL*3), dried over Na₂SO₄, filtered and the filtrate was concentrated under reduced pressure to give a residue. The residue was purified by prep-HPLC (column: Phenomenex Luna C18 75*30 mm*3 um; mobile phase: [H₂O (0.2% FA)-ACN]; gradient: 30%-60% B over 8.0 min) to give the title compound (Compound 170) (66.1 mg, 176 μmol, 50.2% yield, 98.5% purity) as white solid. LCMS (ES⁺): m/z 371.1 [M+H]⁺.

### Example 123. Synthesis of 1-(4-((5-fluoro-4-(3-(oxetan-3-yl)phenyl)pyrimidin-2-yl)amino)piperidin-1-yl)ethan-1-one (Compound 169)

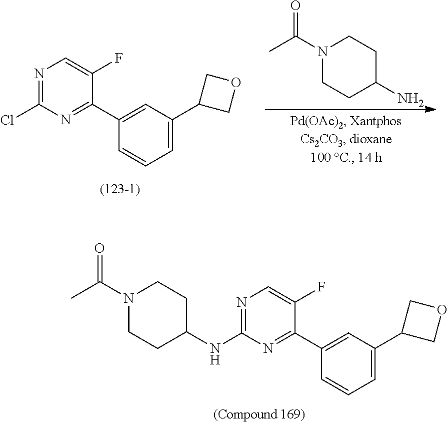

Synthesis of 1-(4-((5-fluoro-4-(3-(oxetan-3-yl)phenyl)pyrimidin-2-yl)amino)piperidin-1-yl)ethan-1-one (Compound 169). To a solution of 2-chloro-5-fluoro-4-(3-(oxetan-3-yl)phenyl)pyrimidine (123-1) (150 mg, 567 umol, 1.00 eq) and 1-(4-aminopiperidin-1-yl)ethan-1-one (80.6 mg, 567 μmol, 1.00 eq) in dioxane (5 mL) were added Cs₂CO₃ (369 mg, 1.13 mmol, 2.00 eq), Xantphos (9.84 mg, 17.0 μmol, 0.0300 eq) and Pd(OAc)₂ (6.36 mg, 28.3 μmol, 0.0500 eq) at 20 °C. The resulting mixture was stirred at 100 °C for 12 h under N₂. LCMS showed 2-chloro-5-fluoro-4-(3-(oxetan-3-yl)phenyl)pyrimidine (123-1) remained and the desired mass was detected. Then 1-(4-aminopiperidin-1-yl)ethan-1-one (80.6 mg, 1.00 eq), Cs₂CO₃ (369 mg, 2.00 eq), Xantphos (9.84 mg, 0.0300 eq) and Pd(OAc)₂ (6.36 mg, 0.0500 eq) were added to the mixture at 20 °C. The mixture was stirred at 100 °C for 2 h under N₂. LCMS showed 2-chloro-5-fluoro-4-(3-(oxetan-3-yl)phenyl)pyrimidine (123-1) was consumed completely and the desired mass was detected. To the mixture was added H₂O (5 mL) and the mixture was extracted with EtOAc (5 mL*3). The combined organic layers were washed with brine (5 mL*3), dried over Na₂SO₄, filtered and the filtrate was concentrated under reduced pressure. This mixture was purified by prep-HPLC (column: Phenomenex Luna C18 75*30 mm*3 um; mobile phase: [H₂O (0.2% FA)-ACN]; gradient: 20%-50% B over 8.0 min) to give the title compound (Compound 169) (15.8 mg, 42.5 umol, 7.49% yield, 99.6% purity) as a white solid. LCMS (ES⁺): m/z 371.1 [M+H]⁺.

### Example 124. Synthesis of N-(trans-4-((5-fluoro-4-(3-(oxetan-3-yl)phenyl)pyrimidin-2-yl)amino)cyclohexyl)acetamide (Compound 175)

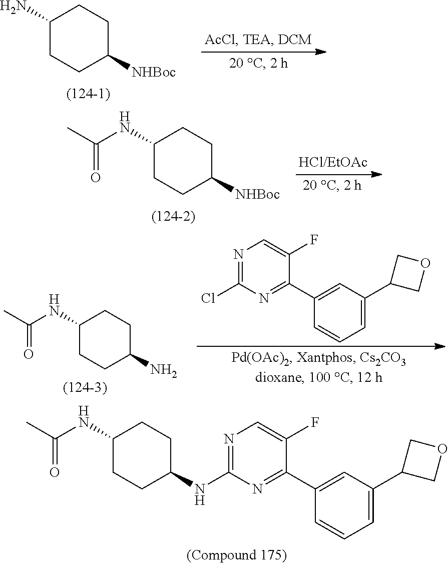 [SMILES: CC(C)(C)OC(=O)N[C@H]1CC[C@H](N)CC1 ; CC(=O)N[C@H]1CC[C@H](NC(=O)OC(C)(C)C)CC1 ; CC(=O)N[C@H]1CC[C@H](N)CC1 ; Fc1cnc(Cl)nc1-c1cccc(C2COC2)c1 ; CC(=O)N[C@H]1CC[C@H](Nc2ncc(F)c(-c3cccc(C4COC4)c3)n2)CC1]

Synthesis of tert-butyl (trans-4-acetamidocyclohexyl)carbamate (124-2). To a solution of tert-butyl (trans-4-aminocyclohexyl)carbamate (124-1) (5.00 g, 23.3 mmol, 1.00 eq) in DCM (50 mL) was added TEA (7.08 g, 69.9 mmol, 9.74 mL, 3.00 eq) at 20 °C, then added acetyl chloride (2.01 g, 25.6 mmol, 1.82 mL, 1.10 eq) at 0 °C. The mixture was stirred at 20 °C for 2 h. LC-MS showed desired mass was detected. The residue was diluted with water 50 mL and extracted with EtOAc (50 mL*2), the combined organic layers were concentrated under reduced pressure to give the title compound (124-2) (5.7 g, crude) as a white solid. LCMS (ES⁺): m/z 201.1 [M+H−56]⁺.

Synthesis of N-(trans-4-aminocyclohexyl)acetamide (124-3). To a solution of to a solution of tert-butyl (trans-4-acetamidocyclohexyl)carbamate (124-2) (1.50 g, 5.85 mmol, 1.00 eq) was added HCl/EtOAc (4 M, 5 mL, 3.42 eq) at 20 °C. The mixture was stirred at 20 °C for 2 h. LC-MS showed desired mass was detected. The reaction mixture was concentrated under reduced pressure to give the title compound (124-3) (1.4 g, crude, HCl) as a white solid. LCMS (ES⁺): m/z 157.1 [M+H]⁺.

Synthesis of N-(trans-4-((5-fluoro-4-(3-(oxetan-3-yl)phenyl)pyrimidin-2-yl)amino)cyclohexyl)acetamide (Compound 175). A mixture of N-(trans-4-aminocyclohexyl)acetamide (124-3) (95.0 mg, 608 μmol, 2.00 eq, HCl), 2-chloro-5-fluoro-4-(3-(oxetan-3-yl)phenyl)pyrimidine (80.4 mg, 304 μmol, 1.00 eq), Cs₂CO₃ (495 mg, 1.52 mmol, 5.00 eq), diacetoxypalladium (6.83 mg, 30.4 μmol, 0.100 eq) and (5-diphenylphosphanyl-9,9-dimethyl-xanthen-4-yl)-diphenyl-phosphane (17.5 mg, 30.4 μmol, 0.100 eq) in dioxane (10 mL) was degassed and purged with N₂ for 3 times at 25 °C, and then the mixture was stirred at 100 °C for 12 h under N₂ atmosphere. LC-MS showed desired mass was detected. The residue was diluted with water 5 mL and extracted with EtOAc (5 mL*2). The combined organic layers were washed with brine (5 mL*2), dried over Na₂SO₄, filtered and concentrated under reduced pressure. The residue was purified by prep-HPLC (column: Phenomenex Luna C18 100*30 mm*3 um; mobile phase: [H₂O (0.2% FA)-ACN]; gradient: 15%-45% B over 8.0 min) to give the title compound (Compound 175) (9.00 mg, 22.9 μmol, 7.55% yield, 98.0% purity) as a white solid. LCMS (ES⁺): m/z 385.1 [M+H]⁺.

### Example 125. Synthesis of trans-4-((5-fluoro-4-(3-(oxetan-3-yl)phenyl)pyrimidin-2-yl)amino)cyclohexane-1-carboxamide (Compound 180)

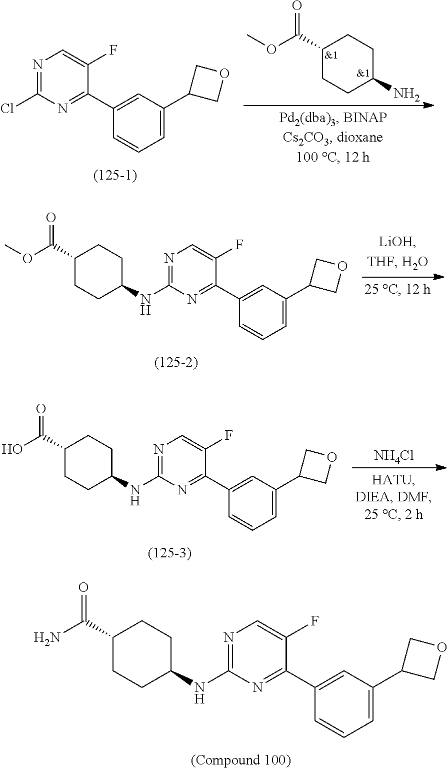 [SMILES: Fc1cnc(Cl)nc1-c1cccc(C2COC2)c1 ; COC(=O)[C@H]1CC[C@H](N)CC1 ; COC(=O)[C@H]1CC[C@H](Nc2ncc(F)c(-c3cccc(C4COC4)c3)n2)CC1 ; O=C(O)[C@H]1CC[C@H](Nc2ncc(F)c(-c3cccc(C4COC4)c3)n2)CC1 ; "NH4Cl" ; NC(=O)[C@H]1CC[C@H](Nc2ncc(F)c(-c3cccc(C4COC4)c3)n2)CC1]

Synthesis of trans-methyl-4-((5-fluoro-4-(3-(oxetan-3-yl)phenyl)pyrimidin-2-yl)amino)cyclohexane-1-carboxylate (125-2). A mixture of 2-chloro-5-fluoro-4-[3-(oxetan-3-yl)phenyl]pyrimidine (125-1) (200 mg, 755 μmol, 1.00 eq), methyl 4-aminocyclohexanecarboxylate (356 mg, 2.27 mmol, 3.00 eq), Cs₂CO₃ (738 mg, 2.27 mmol, 3.00 eq), Pd₂(dba)₃ (69.1 mg, 75.5 μmol, 0.100 eq) and BINAP (47.0 mg, 75.5 μmol, 0.100 eq) in dioxane (8 mL) at 25 °C was degassed and purged with N₂ for 3 times, and then the mixture was stirred at 100 °C for 12 h under N₂ atmosphere. LC-MS showed the desired mass was detected. The reaction mixture was diluted with water 10 mL and extracted with EtOAc (10 mL*3). The combined organic layers dried over Na₂SO₄, filtered and concentrated under reduced pressure. The residue was purified by prep-TLC (SiO₂, Petroleum ether:Ethyl acetate=1:0 to 1:1) to give the title compound (125-2) (500 mg, crude) as a white solid. LCMS (ES⁺): m/z 386.2 [M+H]⁺.

Synthesis of trans-4-((5-fluoro-4-(3-(oxetan-3-yl)phenyl)pyrimidin-2-yl)amino)cyclohexane-1-carboxylic acid (125-3). To a solution of trans-methyl-4-((5-fluoro-4-(3-(oxetan-3-yl)phenyl)pyrimidin-2-yl)amino)cyclohexane-1-carboxylate (125-2) (220 mg, 570 μmol, 1.00 eq) in THF (3 mL) and H₂O (1 mL) was added LiOH (41.0 mg, 1.71 mmol, 3.00 eq) at 25 °C. The mixture was stirred at 25 °C for 12 h. LC-MS showed the desired mass was detected. The mixture was acidified pH-5 with diluted AcOH solution, and filtered to give the title compound (125-3) (74.0 mg, crude) as a white solid. LCMS (ES⁺): m/z 372.1 [M+H]⁺.

Synthesis of trans-4-((5-fluoro-4-(3-(oxetan-3-yl)phenyl)pyrimidin-2-yl)amino)cyclohexane-1-carboxamide (Compound 180). To a solution of trans-4-((5-fluoro-4-(3-(oxetan-3-yl)phenyl)pyrimidin-2-yl)amino)cyclohexane-1-carboxylic acid (125-3) (60.0 mg, 161 μmol, 1.00 eq) in DMF (3 mL) was added DIEA (62.6 mg, 484 μmol, 84.4 μL, 3.00 eq), NH₄Cl (25.9 mg, 484 μmol, 3.00 eq) and HATU (92.1 mg, 242 μmol, 1.50 eq) at 25 °C. The mixture was stirred at 25 °C for 2 h. LC-MS showed the desired mass was detected. The reaction mixture was filtered to give a residue. The residue was purified by prep-HPLC (column: Phenomenex luna C18 150*40 mm*5 um; mobile phase: [H₂O (0.2% FA)-ACN]; gradient: 25%-55% B over 8.0 min) to give the title compound (Compound 180) (36.6 mg, 98.3 μmol, 60.8% yield, 99.5% purity) as a white solid. LCMS (ES⁺): m/z 371.1 [M+H]⁺.

### Example 126. Synthesis of cis-3-((5-fluoro-4-(3-(3-hydroxyoxetan-3-yl)phenyl)pyrimidin-2-yl)amino)cyclohexane-1-carboxamide (Compound 173)

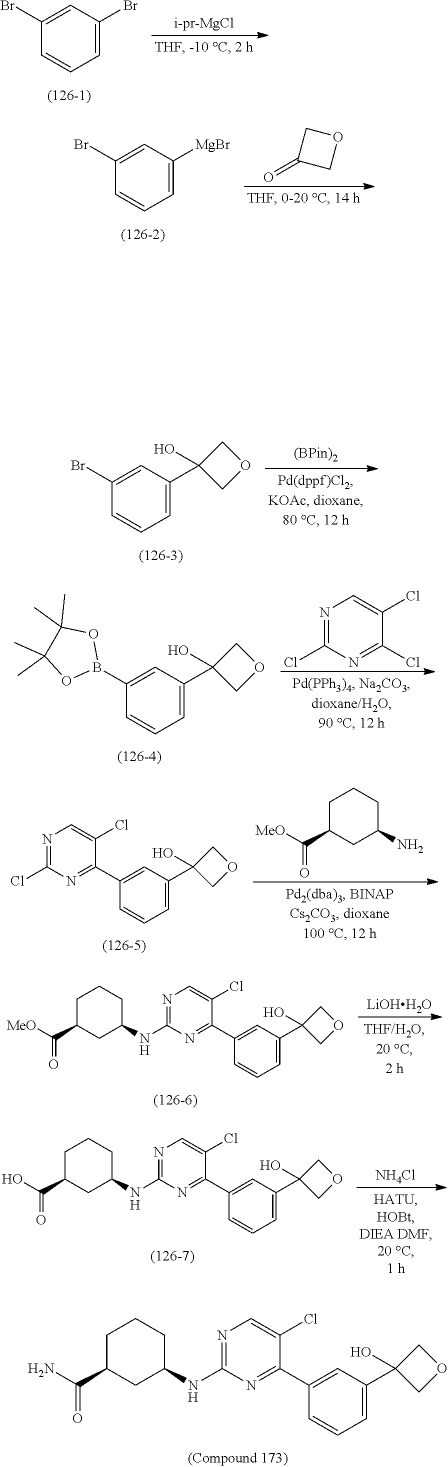

Synthesis of (3-bromophenyl)magnesium bromide (126-2). To a solution of 1,3-dibromobenzene (126-1) (10.0 g, 42.4 mmol, 5.12 mL, 1.00 eq) in THF (100 mL) was added i-Pr—MgCl (2.00 M, 18.2 mL, 0.860 eq) at −10 °C under N₂. The mixture was stirred at −10 °C for 2 h under N₂. TLC (SiO₂, Petroleum ether:Ethyl acetate=3:1) showed 1,3-dibromobenzene (126-1) was remained and one new spot formed. Then the mixture was used to next step directly, the title compound (126-2) (11.0 g, crude) was in THF (100 mL) as a yellow liquid. (used directly in the solvent, so no HNMR spectra)

Synthesis of 3-(3-bromophenyl)oxetan-3-ol (126-3). oxetan-3-one (2.00 g, 27.8 mmol, 1.00 eq) was added to the mixture of (3-bromophenyl)magnesium bromide (126-2) (0.5 M, 44.4 mL, 0.800 eq) in THF (100 mL), the mixture was stirred at 0 °C for 2 h, then the mixture was stirred at 20 °C for another 12 h. TLC (SiO₂, Petroleum ether:Ethyl acetate=3:1) showed (3-bromophenyl)magnesium bromide (126-2) was consumed and three new spots formed. The mixture was quenched by sat.NH₄Cl (100 mL), then the mixture was extracted by EtOAc (100 mL*3), the combined organic layers were washed with brine (80 mL), dried over Na₂SO₄, then filtered and the filtrate was concentrated in vacuo to give the crude product. The crude product was purified by MPLC (SiO₂, Petroleum ether:Ethyl acetate=1/0 to 3/1) to give the title compound (126-3) (3.80 g, 16.6 mmol, 29.9% yield) as a white solid. The product couldn't detected by LCMS. It was confirmed by ¹H NMR (400 MHz, DMSO-d₆) δ=7.76 (s, 1H), 7.65 (d, J=7.8 Hz, 1H), 7.51 (dd, J=0.9, 7.9 Hz, 1H), 7.41-7.35 (m, 1H), 6.51 (s, 1H), 4.78 (d, J=6.6 Hz, 2H), 4.67 (d, J=6.8 Hz, 2H)

Synthesis of 3-(3-(4,4,5,5-tetramethyl-1,3,2-dioxaborolan-2-yl)phenyl)oxetan-3-ol (126-4). To a solution of 3-(3-bromophenyl)oxetan-3-ol (126-3) (3.00 g, 13.1 mmol, 1.00 eq) and 4,4,5,5-tetramethyl-2-(4,4,5,5-tetramethyl-1,3,2-dioxaborolan-2-yl)-1,3,2-dioxaborolane (4.99 g, 19.6 mmol, 1.50 eq) in dioxane (80 mL) was added Pd(dppf)Cl₂ (479 mg, 655 μmol, 0.0500 eq) and KOAc (2.57 g, 26.2 mmol, 2.00 eq) at 20 °C, then the mixture was stirred at 80 °C for 12 h under N₂. TLC (SiO₂, Petroleum ether:Ethyl acetate=1:1) showed 3-(3-bromophenyl)oxetan-3-ol (126-3) was consumed and three new spots formed. H₂O (80 mL) was added to the mixture and the mixture was extracted with EtOAc (80 mL*3), the combined organic layers were washed with brine (80 mL*3), dried over Na₂SO₄, filtered and the filtrate was concentrated in vacuo to give the crude product. The crude product was purified by MPLC (SiO₂, Petroleum ether:Ethyl acetate=1/0 to 1/1) to give the title compound (126-4) (3.60 g, 13.0 mmol, 99.6% yield) as a yellow solid. The product couldn't detected by LCMS. It was confirmed by ¹H NMR (400 MHz, CHLOROFORM-d) δ=7.93 (d, J=0.6 Hz, 1H), 7.73-7.68 (m, 1H), 7.62-7.57 (m, 1H), 7.38-7.31 (m, 1H), 4.91-4.86 (m, 2H), 4.83-4.80 (m, 2H), 1.28-1.26 (m, 12H)

Synthesis of 3-(3-(2-chloro-5-fluoropyrimidin-4-yl)phenyl)oxetan-3-ol (126-5). To a solution of 3-(3-(4,4,5,5-tetramethyl-1,3,2-dioxaborolan-2-yl)phenyl)oxetan-3-ol (126-4) (3.60 g, 13.0 mmol, 1.00 eq) and 2,4-dichloro-5-fluoropyrimidine (2.18 g, 13.0 mmol, 1.00 eq) in dioxane (50 mL) and H₂O (5 mL) were added Na₂CO₃ (2.76 g, 26.1 mmol, 2.00 eq) and Pd(PPh₃)₄ (301 mg, 261 μmol, 0.0200 eq) at 20 °C. The mixture was stirred at 90 °C for 12 h under N₂ atmosphere. LCMS showed the desired mass was detected. H₂O (50 mL) was added to the mixture and the mixture was extracted with EtOAc (50 mL*3), the combined organic layers were washed with brine (50 mL*3), dried over Na₂SO₄, filtered and the filtrate was concentrated in vacuo to give the crude product. The crude product was purified by MPLC (SiO₂, Petroleum ether:Ethyl acetate=1/0 to 2/1) to give the title compound (126-5) (3.80 g, crude) as a colorless oil. LCMS (ES⁺): m/z 281.0 [M+H]⁺ (comments: after purified, it was not clean enough, it was crude product and exceed theoretical yield, so there are no mmol and yield)

Synthesis of cis-methyl 3-((5-fluoro-4-(3-(3-hydroxyoxetan-3-yl)phenyl)pyrimidin-2-yl)amino)cyclohexane-1-carboxylate (126-6). To a solution of 3-(3-(2-chloro-5-fluoropyrimidin-4-yl)phenyl)oxetan-3-ol (126-5) (500 mg, 1.78 mmol, 1.00 eq) and cis-methyl 3-aminocyclohexane-1-carboxylate (345 mg, 1.78 mmol, 1.00 eq, HCl) in dioxane (10 mL) were added BINAP (66.6 mg, 107 μmol, 0.0600 eq), Cs₂CO₃ (1.16 g, 3.56 mmol, 2.00 eq) and Pd₂(dba)₃ (65.3 mg, 71.3 μmol, 0.0400 eq) at 20 °C, the mixture was stirred at 100 °C for 12 h under N₂ atmosphere. LCMS showed 3-(3-(2-chloro-5-fluoropyrimidin-4-yl)phenyl)oxetan-3-ol (126-5) was consumed and the desired mass was detected. H₂O (10 mL) was added to the mixture and the mixture was extracted with EtOAc (10 mL*3), the combined organic layers were washed with brine (10 mL*3), dried over Na₂SO₄, filtered and the filtrate was concentrated in vacuo to give the crude product. The crude product was purified by prep-TLC (SiO₂, Petroleum ether:Ethyl acetate=1:1) to give the title compound (126-6) (230 mg, 573 μmol, 32.2% yield) as a colorless oil. LCMS (ES⁺): m/z 402.2 [M+H]⁺

Synthesis of cis-3-((5-fluoro-4-(3-(3-hydroxyoxetan-3-yl)phenyl)pyrimidin-2-yl)amino)cyclohexane-1-carboxylic acid (126-7). To a solution of cis-methyl 3-((5-fluoro-4-(3-(3-hydroxyoxetan-3-yl)phenyl)pyrimidin-2-yl)amino)cyclohexane-1-carboxylate (126-6) (230 mg, 573 μmol, 1.00 eq) in THF (2 mL) and H₂O (1 mL) was added LiOH·H₂O (48.1 mg, 1.15 mmol, 2.00 eq) at 20 °C, the mixture was stirred at 20 °C for 2 h. LCMS showed cis-methyl 3-((5-fluoro-4-(3-(3-hydroxyoxetan-3-yl)phenyl)pyrimidin-2-yl)amino)cyclohexane-1-carboxylate (126-6) was consumed and the desired mass was detected. The reaction mixture was concentrated in vacuo to remove THF. HCl (1 M) was added to the aqueous to adjust the pH to 2. Then the mixture was extracted with EtOAc (3 mL*3). The combined organic layers were dried over Na₂SO₄, filtered and the filtrate was concentrated in vacuo to give the title compound (126-7) (148 mg, crude) as a yellow oil. LCMS (ES⁺): m/z 388.2 [M+H]⁺

Synthesis of cis-3-((5-fluoro-4-(3-(3-hydroxyoxetan-3-yl)phenyl)pyrimidin-2-yl)amino)cyclohexane-1-carboxamide (Compound 173). To a solution of cis-3-((5-fluoro-4-(3-(3-hydroxyoxetan-3-yl)phenyl)pyrimidin-2-yl)amino)cyclohexane-1-carboxylic acid (126-7) (128 mg, 330 μmol, 1.00 eq) and NH₄Cl (70.7 mg, 1.32 mmol, 4.00 eq) in DMF (1 mL) was added DIEA (214 mg, 1.65 mmol, 288 μL, 5.00 eq), HATU (151 mg, 396 μmol, 1.20 eq), HOBt (22.3 mg, 165 μmol, 0.500 eq) at 20 °C, then the mixture was stirred at 20 °C for 1 h. LCMS showed cis-3-((5-fluoro-4-(3-(3-hydroxyoxetan-3-yl)phenyl)pyrimidin-2-yl)amino)cyclohexane-1-carboxylic acid (126-7) was remained and the desired mass was detected. The mixture was concentrated in vacuo to give the crude product. The crude product was purified by prep-HPLC (column: Phenomenex Luna C18 100*30 mm*3 um; mobile phase: [H₂O (0.2% FA)-ACN]; gradient: 25%-60% B over 8.0 min) to give the title compound (Compound 173) (15.3 mg, 35.8 μmol, 10.8% yield, 90.3% purity) as a white solid. LCMS (ES⁺): m/z 387.2 [M+H]⁺.

### Example 127. Synthesis of 1-(4-((5-fluoro-4-(3-(3-hydroxyoxetan-3-yl)phenyl)pyrimidin-2-yl)amino)piperidin-1-yl)ethan-1-one (Compound 171)

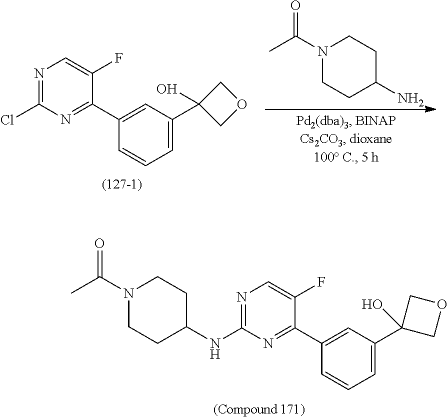

Synthesis of 1-(4-((5-fluoro-4-(3-(3-hydroxyoxetan-3-yl)phenyl)pyrimidin-2-yl)amino)piperidin-1-yl)ethan-1-one (Compound 171). To a solution of 3-(3-(2-chloro-5-fluoropyrimidin-4-yl)phenyl)oxetan-3-ol (127-1) (30.0 mg, 107 μmol, 1.00 eq) and 1-(4-aminopiperidin-1-yl)ethan-1-one (15.2 mg, 107 μmol, 1.00 eq) in dioxane (1 mL) were added BINAP (3.99 mg, 6.41 μmol, 0.0600 eq), Cs₂CO₃ (69.7 mg, 214 μmol, 2.00 eq) and Pd₂(dba)₃ (3.91 mg, 4.28 μmol, 0.0400 eq) at 20 °C, then the mixture was stirred at 100 °C for 2 h under N₂ atmosphere. LCMS showed 3-(3-(2-chloro-5-fluoropyrimidin-4-yl)phenyl)oxetan-3-ol (127-1) was remained and the desired mass was detected. Then the mixture was stirred at 100 °C for another 3 h under N₂ atmosphere. LCMS showed 3-(3-(2-chloro-5-fluoropyrimidin-4-yl)phenyl)oxetan-3-ol (127-1) was consumed and the desired mass was detected. H₂O (2 mL) was added to the mixture and the mixture was extracted with EtOAc (2 mL*3), the combined organic layers were washed with brine (2 mL*3), dried over Na₂SO₄, filtered and the filtrate was concentrated in vacuo to give the crude product. The crude product was purified by prep-HPLC (column: Phenomenex Luna C18 100*30 mm*3 um; mobile phase: [1H₂O (0.2% FA)-ACN]; gradient: 10%-40% B over 8.0 min) to give the title compound (Compound 171) (6.80 mg, 17.5 μmol, 16.4% yield, 99.5% purity) as a white solid. LCMS (ES⁺): m/z 387.1 [M+H]⁺.

### Example 128. Synthesis of N-(trans-4-((5-fluoro-4-(3-(3-hydroxyoxetan-3-yl)phenyl)pyrimidin-2-yl)amino)cyclohexyl)acetamide (Compound 176)

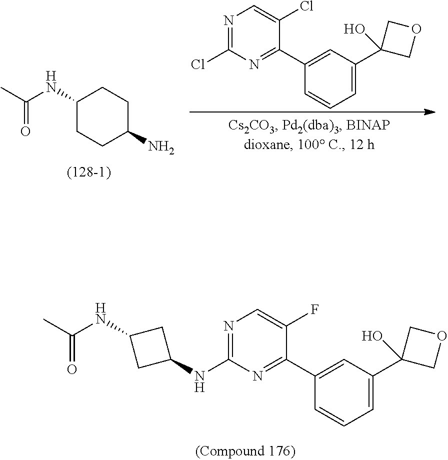 [SMILES: CC(=O)N[C@H]1CC[C@H](N)CC1 ; OC1(c2cccc(-c3nc(Cl)ncc3Cl)c2)COC1 ; CC(=O)N[C@H]1C[C@H](Nc2ncc(F)c(-c3cccc(C4(O)COC4)c3)n2)C1]

Synthesis of N-(trans-4-((5-fluoro-4-(3-(3-hydroxyoxetan-3-yl)phenyl)pyrimidin-2-yl)amino)cyclohexyl)acetamide (Compound 176). A mixture of N-(trans-4-aminocyclohexyl)acetamide (128-1) (222 mg, 1.43 mmol, 4.00 eq), 3-(3-(2-chloro-5-fluoropyrimidin-4-yl)phenyl)oxetan-3-ol (100 mg, 356 μmol, 1.00 eq), Cs₂CO₃ (1.16 g, 3.56 mmol, 10.0 eq), Pd₂(dba)₃ (65.2 mg, 71.2 μmol, 0.200 eq) and BINAP (44.3 mg, 71.2 μmol, 0.200 eq) in dioxane (8 mL) was degassed and purged with N₂ for 3 times at 25 °C, and then the mixture was stirred at 100 °C for 12 h under N₂ atmosphere. LC-MS showed desired mass was detected. The residue was diluted with water 5 mL and extracted with EtOAc 5 mL (5 mL*2). The combined organic layers were washed with brine 5 mL (5 mL*2), dried over Na₂SO₄, filtered and concentrated under reduced pressure to give a residue. The residue was purified by prep-HPLC (column: Phenomenex Luna C18 100*30 mm*3 um; mobile phase: [H₂O (0.2% FA)-ACN]; gradient: 10%-50% B over 8.0 min) to give the title compound (Compound 176) (10 mg, 24.42 μmol, 6.85% yield, 97.78% purity) as a white solid. LCMS (ES⁺): m/z 401.1 [M+H]⁺.

### Example 129. Synthesis of trans-4-((5-fluoro-4-(3-(3-hydroxyoxetan-3-yl)phenyl)pyrimidin-2-yl)amino)cyclohexane-1-carboxamide (Compound 181)

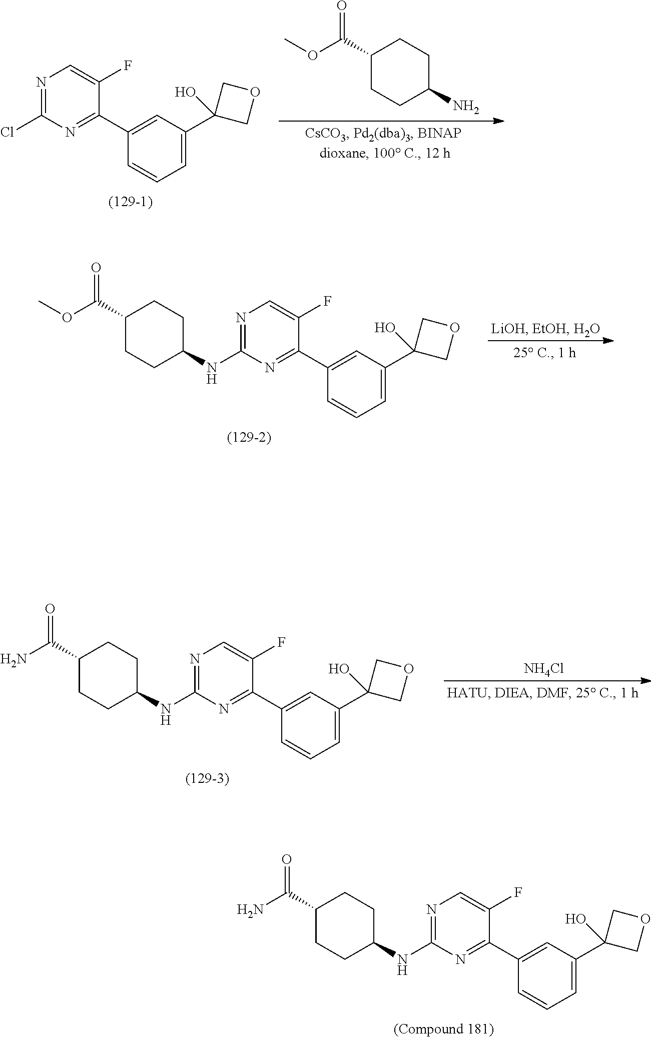

Synthesis of trans-methyl 4-[[5-fluoro-4-[3-(3-hydroxyoxetan-3-yl)phenyl]pyrimidin-2-yl]amino]cyclohexanecarboxylate (129-2). A mixture of 3-[3-(2-chloro-5-fluoro-pyrimidin-4-yl)phenyl]oxetan-3-ol (129-1) (400 mg, 1.43 mmol, 1.00 eq), trans-methyl 4-aminocyclohexanecarboxylate (336 mg, 2.14 mmol, 1.50 eq), Cs₂CO₃ (1.39 g, 4.28 mmol, 3.00 eq), Pd₂(dba)₃ (130 mg, 142 μmol, 0.100 eq) and BINAP (88.7 mg, 142 mol, 0.100 eq) in dioxane (6 mL) was degassed and purged with N₂ for 3 times at 25 °C, and then the mixture was stirred at 100 °C for 12 h under N₂ atmosphere. LC-MS showed the desired mass was detected. The reaction mixture was diluted with H₂O 20 mL and extracted with EtOAc (50 mL*3). The combined organic layers were concentrated under reduced pressure. The residue was purified by column chromatography (SiO₂, Petroleum ether:Ethyl acetate=1:0 to 1:1). to give the title compound (129-2) (300 mg, 735 μmol, 51.6% yield, 98.4% purity) as a yellow solid. LCMS (ES⁺): m/z 402.3 [M+H]⁺.

Synthesis of trans-4-((5-fluoro-4-(3-(3-hydroxyoxetan-3-yl)phenyl)pyrimidin-2-yl)amino)cyclohexane-1-carboxylic acid (129-3). To a solution of trans-methyl 4-[[5-fluoro-4-j[3-(3-hydroxyoxetan-3-yl)phenyl]pyrimidin-2-yl]amino]cyclohexanecarboxylate (129-2) (250 mg, 622 μmol, 1.00 eq) in EtOH (5 mL) and H₂O (1 mL) was added LiOH (29.8 mg, 1.25 mmol, 2.00 eq) at 25 °C. The mixture was stirred at 25 °C for 1 h. LC-MS showed the desired mass was detected. The mixture was acidified pH-5 with diluted AcOH solution, and filtered to give the title compound (129-3) (200 mg, crude) as a yellow solid. LCMS (ES⁺): m/z 388.2 [M+H]⁺.

Synthesis of trans-4-((5-fluoro-4-(3-(3-hydroxyoxetan-3-yl)phenyl)pyrimidin-2-yl)amino)cyclohexane-1-carboxamide (Compound 181). A To a solution of trans-4-[[5-fluoro-4-[3-(3-hydroxyoxetan-3-yl)phenyl]pyrimidin-2-yl]amino]cyclohexanecarboxylic acid (129-3) (160 mg, 413 μmol, 1.00 eq) in DMF (5 mL) was added NH₄Cl (44.1 mg, 826 μmol, 2.00 eq), HATU (235 mg, 619 μmol, 1.50 eq) and DIEA (160 mg, 1.24 mmol, 215 μL, 3.00 eq) at 25 °C. The mixture was stirred at 25 °C for 1 h. LC-MS showed the desired mass was detected. The reaction mixture was concentrated under reduced pressure. The residue was purified by prep-HPLC (column: Phenomenex Luna C18 100*30 mm*3 um; mobile phase: [H₂O (0.2% FA)-ACN]; gradient: 15%-45% B over 8.0 min) to give the title compound (Compound 181) (102 mg, 259 μmol, 62.9% yield, 97.6% purity) as a white solid. LCMS (ES⁺): m/z 387.1 [M+H]⁺.

### Example 130. Synthesis of cis-3-((5-fluoro-4-(2-(2-hydroxypropan-2-yl)pyridin-4-yl)pyrimidin-2-yl)amino)cyclohexane-1-carboxamide (Compound 177)

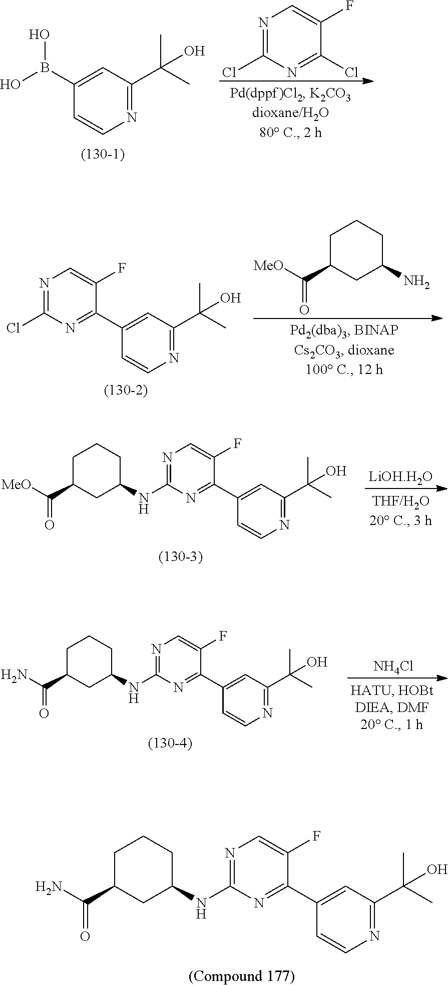

Synthesis of 2-(4-(2-chloro-5-fluoropyrimidin-4-yl)pyridin-2-yl)propan-2-ol (130-2). To a solution of [2-(1-hydroxy-1-methyl-ethyl)-4-pyridyl]boronic acid (130-1) (1.00 g, 5.52 mmol, 1.00 eq) in dioxane (10 mL) and H₂O (2 mL) were added 2,4-dichloro-5-fluoropyrimidine (1.11 g, 6.63 mmol, 1.20 eq), Pd(dppf)Cl₂ (404 mg, 553 μmol, 0.100 eq) and K₂CO₃ (2.29 g, 16.6 mmol, 3.00 eq) at 20 °C under N₂ atmosphere. The mixture was stirred at 80 °C for 2 h under N₂ atmosphere. LCMS showed the desired target MS was detected. H₂O (15 mL) was added to the solution, the mixture was extracted with EtOAc (15 mL*3), then the combined organic lays were dried over Na₂SO₄, filtered and the filtrate was concentrated in vacuo to give the crude product. The crude product was purified by MPLC (SiO₂, Petroleum ether:Ethyl acetate=1:0 to 1:1) to give the title compound (130-2) (570 mg, 2.13 mmol, 38.5% yield) as a yellow oil. LCMS (ES⁺): m/z 268.0 [M+H]⁺.

Synthesis of cis-methyl 3-((5-fluoro-4-(2-(2-hydroxypropan-2-yl)pyridin-4-yl)pyrimidin-2-yl)amino)cyclohexane-1-carboxylate (130-3). To a solution of 2-(4-(2-chloro-5-fluoropyrimidin-4-yl)pyridin-2-yl)propan-2-ol (130-2) (550 mg, 2.05 mmol, 1.00 eq) and cis-methyl 3-aminocyclohexane-1-carboxylate (398 mg, 2.05 mmol, 1.00 eq, HCl) in dioxane (9 mL) were added BINAP (89.6 mg, 144 μmol, 0.0700 eq), Pd₂(dba)₃ (94.1 mg, 103 μmol, 0.0500 eq), Cs₂CO₃ (2.01 g, 6.16 mmol, 3.00 eq) at 20 °C, the mixture was stirred at 100 °C for 12 h under N₂ atmosphere. LCMS showed 2-(4-(2-chloro-5-fluoropyrimidin-4-yl)pyridin-2-yl)propan-2-ol (130-2) was consumed and the desired target MS was detected. H₂O (9 mL) was added to the mixture and the mixture was extracted with EtOAc (9 mL*3), the combined organic layers were washed with brine (9 mL), dried over Na₂SO₄, filtered and the filtrate was concentrated in vacuo to give the crude product. The mixture was purified by prep-TLC (SiO₂, PE:EtOAc=3:1) to give the title compound (130-3) (280 mg, 721 μmol, 35.1% yield) as a yellow solid. LCMS (ES⁺): m/z 389.2 [M+H]⁺.

Synthesis of cis-3-((5-fluoro-4-(2-(2-hydroxypropan-2-yl)pyridin-4-yl)pyrimidin-2-yl)amino)cyclohexane-1-carboxylic acid (130-4). To a solution of cis-methyl 3-((5-fluoro-4-(2-(2-hydroxypropan-2-yl)pyridin-4-yl)pyrimidin-2-yl)amino)cyclohexane-1-carboxylate (130-3) (280 mg, 721 μmol, 1.00 eq) in THF (1.5 mL) and H₂O (0.75 mL) was added LiOH·H₂O (60.5 mg, 1.44 mmol, 2.00 eq) at 20 °C, the mixture was stirred at 20 °C for 2 h. LCMS showed cis-methyl 3-((5-fluoro-4-(2-(2-hydroxypropan-2-yl)pyridin-4-yl)pyrimidin-2-yl)amino)cyclohexane-1-carboxylate (130-3) remained and the desired target MS was detected. Then the mixture was stirred at 20 °C for another 1 h. LCMS showed cis-methyl 3-((5-fluoro-4-(2-(2-hydroxypropan-2-yl)pyridin-4-yl)pyrimidin-2-yl)amino)cyclohexane-1-carboxylate (130-3) was consumed completely and the desired target MS was detected. The mixture was concentrated in vacuo to remove THF, then the remaining aqueous phase was adjust pH to 4 by HCl (1M), then white solid was precipitated, the mixture was filtered and the filter cake was dried in vacuo to give the title compound (130-4) (190 mg, 507 μmol, 70.4% yield) as a white solid. LCMS (ES⁺): m/z 375.2 [M+H]⁺.

Synthesis of cis-3-((5-fluoro-4-(2-(2-hydroxypropan-2-yl)pyridin-4-yl)pyrimidin-2-yl)amino)cyclohexane-1-carboxamide (Compound 177). To a solution of cis-3-((5-fluoro-4-(2-(2-hydroxypropan-2-yl)pyridin-4-yl)pyrimidin-2-yl)amino)cyclohexane-1-carboxylic acid (130-4) (180 mg, 481 μmol, 1.00 eq) and NH₄Cl (103 mg, 1.92 mmol, 4.00 eq) in DMF (2 mL) were added HATU (219 mg, 577 μmol, 1.20 eq), HOBt (32.5 mg, 240 μmol, 0.500 eq), DIEA (311 mg, 2.40 mmol, 419 μL, 5.00 eq) at 20 °C, then the mixture was stirred at 20 °C for 1 h. LCMS showed cis-3-((5-fluoro-4-(2-(2-hydroxypropan-2-yl)pyridin-4-yl)pyrimidin-2-yl)amino)cyclohexane-1-carboxylic acid (130-4) was consumed and the desired target MS was detected. The organic layers were filtered and the filtrate was concentrated under reduced pressure to give a residue. The residue was purified by prep-HPLC (column: 2_Phenomenex Gemini C18 75*40 mm*3 um; mobile phase: [H₂O (10 mM NH₄HCO₃)-ACN]; gradient: 25%-55% B over 8.0 min) to give the title compound (Compound 177) (22.3 mg, 58.6 μmol, 12.2% yield, 98.2% purity) as a white solid. LCMS (ES⁺): m/z 374.1 [M+H]⁺.

### Example 131. Synthesis of 1-(4-((5-fluoro-4-(2-(2-hydroxypropan-2-yl)pyridin-4-yl)pyrimidin-2-yl)amino)piperidin-1-yl)ethan-1-one (Compound 172)

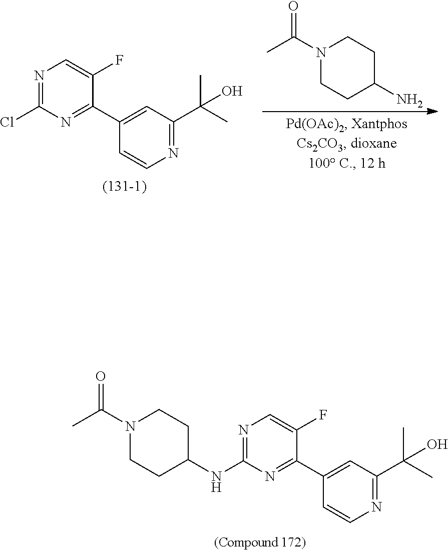

Synthesis of 1-(4-((5-fluoro-4-(2-(2-hydroxypropan-2-yl)pyridin-4-yl)pyrimidin-2-yl)amino)piperidin-1-yl)ethan-1-one (Compound 172). To a solution of 2-(4-(2-chloro-5-fluoropyrimidin-4-yl)pyridin-2-yl)propan-2-ol (131-1) (200 mg, 747 μmol, 1.00 eq) and 1-(4-aminopiperidin-1-yl)ethan-1-one (106 mg, 747 μmol, 1.00 eq) in dioxane (3 mL) were added Xantphos (13.0 mg, 22.4 μmol, 0.0300 eq), Pd(OAc)₂ (8.39 mg, 37.4 μmol, 0.0500 eq), Cs₂CO₃ (487 mg, 1.49 mmol, 2.00 eq) at 20 °C, the mixture was stirred at 100 °C for 12 h under N₂ atmosphere. LCMS showed 2-(4-(2-chloro-5-fluoropyrimidin-4-yl)pyridin-2-yl)propan-2-ol (131-1) was consumed and the desired target MS was detected. The organic layers were filtered and the filtrate was concentrated under reduced pressure to give a residue. The residue was purified by prep-HPLC (column: Phenomenex luna C18 100*40 mm*3 um; mobile phase: [H₂O (0.2% FA)-ACN]; gradient: 10%-45% B over 8.0 min) to give the title compound (Compound 172) (20.1 mg, 52.0 μmol, 6.95% yield, 96.5% purity) as a white solid. LCMS (ES⁺): m/z 374.1 [M+H]⁺.

### Example 132. Synthesis of trans-N-(4-((5-fluoro-4-(2-(2-hydroxypropan-2-yl)pyridin-4-yl)pyrimidin-2-yl)amino)cyclohexyl)acetamide (Compound 241)

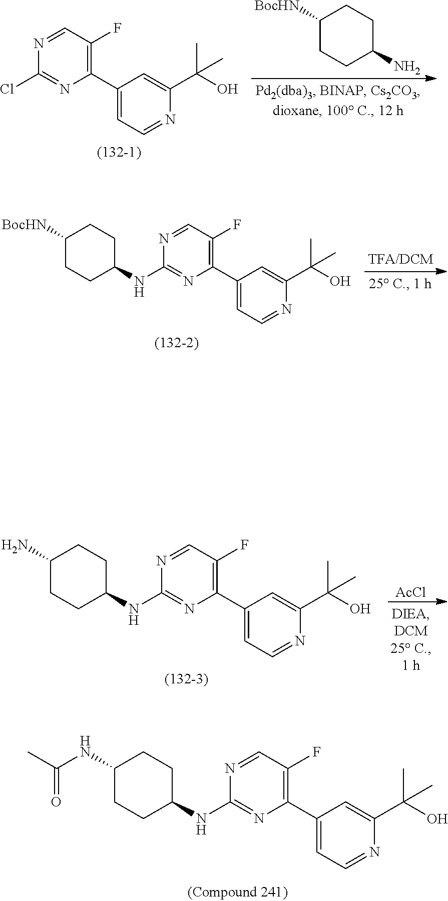

Synthesis of trans-tert-butyl (4-((5-fluoro-4-(2-(2-hydroxypropan-2-yl)pyridin-4-yl)pyrimidin-2-yl)amino)cyclohexyl)carbamate (132-2). To a solution of 2-(4-(2-chloro-5-fluoropyrimidin-4-yl)pyridin-2-yl)propan-2-ol (132-1) (1.00 g, 3.74 mmol, 1.00 eq) in dioxane (18 mL) were added BINAP (140 mg, 224 μmol, 0.0600 eq), Cs₂CO₃ (2.43 g, 7.47 mmol, 2.00 eq), Pd₂(dba)₃ (103 mg, 112 μmol, 0.0300 eq) and trans-tert-butyl N-(4-aminocyclohexyl)carbamate (801 mg, 3.74 mmol, 1.00 eq) at 25 °C under N₂ atmosphere. The mixture was stirred at 100 °C for 12 h under N₂. LCMS showed the desired mass was detected. Water (15 mL) was added to the mixture at 20 °C. Then the reaction mixture was extracted with EtOAc (12 mL*3). The combined organic layers were dried over anhydrous sodium sulfate, filtered and the filtrate was concentrated under reduced pressure to give a residue. The residue was purified by column chromatography (SiO₂, Petroleum ether:Ethyl acetate=2:1 to 1:1) to give the title compound (132-2) (520 mg, 1.17 mmol, 31.2% yield) as a yellow oil. LCMS (ES⁺): m/z 446.2 [M+H]⁺.

Synthesis of trans-2-(4-(2-((4-aminocyclohexyl)amino)-5-fluoropyrimidin-4-yl)pyridin-2-yl)propan-2-ol (132-3). To a solution of trans-tert-butyl (4-((5-fluoro-4-(2-(2-hydroxypropan-2-yl)pyridin-4-yl)pyrimidin-2-yl)amino)cyclohexyl)carbamate (132-3) (520 mg, 1.17 mmol, 1.00 eq) in DCM (6 mL) was added TFA (2 mL) at 25 °C. The mixture was stirred at 25 °C for 1 h. LCMS showed trans-tert-butyl (4-((5-fluoro-4-(2-(2-hydroxypropan-2-yl)pyridin-4-yl)pyrimidin-2-yl)amino)cyclohexyl)carbamate (132-3) was consumed completely and the desired mass was detected. The reaction mixture was concentrated under reduced pressure to give the title compound (132-5) (530 mg, crude, TFA) as a yellow oil. LCMS (ES⁺): m/z 346.1 [M+H]⁺.

Synthesis of trans-N-(4-((5-fluoro-4-(2-(2-hydroxypropan-2-yl)pyridin-4-yl)pyrimidin-2-yl)amino)cyclohexyl)acetamide (Compound 241). To a solution of trans-2-(4-(2-((4-aminocyclohexyl)amino)-5-fluoropyrimidin-4-yl)pyridin-2-yl)propan-2-ol (132-5) (300 mg, 653 μmol, 1.00 eq, TFA) in DCM (4 mL) were added DIEA (253 mg, 1.96 mmol, 341 μL, 3.00 eq) and acetyl chloride (41.0 mg, 522 μmol, 37.1 μL, 0.800 eq) at 0 °C. The mixture was stirred at 25 °C for 1 h. LCMS showed the desired mass was detected. H2O (0.1 mL) was added to the mixture to quench acetyl chloride, then the reaction mixture was concentrated under reduced pressure to remove solvent to give a residue. The residue was purified by prep-HPLC (column: Phenomenex luna C18 100*40 mm*3 um; mobile phase: [H₂O (0.2% FA)-ACN]; gradient: 1%-30% B over 8.0 min) to give the title compound (Compound 241) (74.1 mg, 191 μmol, 29.3% yield, 100% purity) as a white solid. LCMS (ES⁺): m/z 388.2 [M+H]⁺.

### Example 133. Synthesis of trans-4-((5-fluoro-4-(2-(2-hydroxypropan-2-yl)pyridin-4-yl)pyrimidin-2-yl)amino)cyclohexane-1-carboxamide (Compound 242)

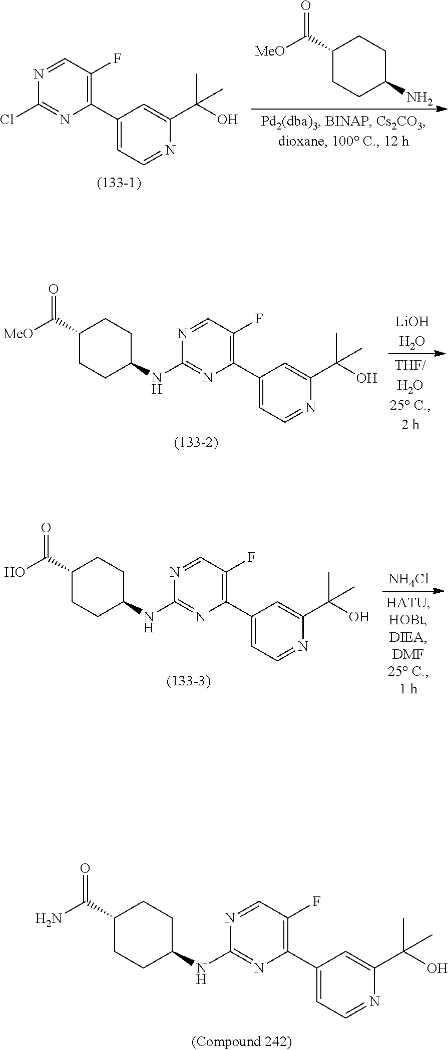

Synthesis of trans-methyl-4-((5-fluoro-4-(2-(2-hydroxypropan-2-yl)pyridin-4-yl)pyrimidin-2-yl)amino)cyclohexane-1-carboxylate (133-2). To a solution of 2-(4-(2-chloro-5-fluoropyrimidin-4-yl)pyridin-2-yl)propan-2-ol (133-1) (1.00 g, 3.74 mmol, 1.00 eq) in dioxane (15 mL) were added BINAP (140 mg, 224 μmol, 0.0600 eq), Cs₂CO₃ (2.43 g, 7.47 mmol, 2.00 eq), Pd₂(dba)₃ (103 mg, 112 μmol, 0.0300 eq) and trans-methyl 4-aminocyclohexanecarboxylate (587 mg, 3.74 mmol, 1.00 eq) at 25 °C under N₂ atmosphere. The mixture was stirred at 100 °C for 12 h under N₂. LCMS showed the desired mass was detected. Water (20 mL) was added to the mixture at 20 °C. Then the reaction mixture was extracted with EtOAc (15 mL*3). The combined organic layers were dried over anhydrous sodium sulfate, filtered and the filtrate was concentrated under reduced pressure to give a residue. The residue was purified by column chromatography (SiO₂, Petroleum ether:Ethyl acetate=2:1 to 1:1) to give the title compound (133-2) (370 mg, 953 μmol, 25.5% yield) as a yellow oil. LCMS (ES⁺): m/z 389.1 [M+H]⁺.

Synthesis of trans-4-((5-fluoro-4-(2-(2-hydroxypropan-2-yl)pyridin-4-yl)pyrimidin-2-yl)amino)cyclohexane-J-carboxylic acid (133-3). To a solution of methyl trans-4-((5-fluoro-4-(2-(2-hydroxypropan-2-yl)pyridin-4-yl)pyrimidin-2-yl)amino)cyclohexane-1-carboxylate (133-2) (370 mg, 953 μmol, 1.00 eq) in THF (8 mL) and H₂O (4 mL) was added LiOH·H₂O (160 mg, 3.81 mmol, 4.00 eq) at 25 °C. The mixture was stirred at 25 °C for 2 h. LCMS showed methyl trans-4-((5-fluoro-4-(2-(2-hydroxypropan-2-yl)pyridin-4-yl)pyrimidin-2-yl)amino)cyclohexane-1-carboxylate (133-2) was consumed completely and the desired mass was detected. The reaction mixture was concentrated under reduced pressure to remove THF. Then HCl (1M) was added dropwise to the aqueous phase until the pH had reached 3. The above aqueous solution was extracted with EtOAc (5 mL*3). The combined organic layers were dried over anhydrous Na₂SO₄, filtered and the filtrate was concentrated under reduced pressure to give the title compound (133-3) (350 mg, crude) as a yellow oil. LCMS (ES⁺): m/z 375.1 [M+H]⁺.

Synthesis of trans-4-((5-fluoro-4-(2-(2-hydroxypropan-2-yl)pyridin-4-yl)pyrimidin-2-yl)amino)cyclohexane-1-carboxamide (Compound 242). To a solution of trans-4-((5-fluoro-4-(2-(2-hydroxypropan-2-yl)pyridin-4-yl)pyrimidin-2-yl)amino)cyclohexane-1-carboxylic acid (133-3) (330 mg, 881 μmol, 1.00 eq) in DMF (3 mL) were added NH₄Cl (189 mg, 3.53 mmol, 4.00 eq), HATU (402 mg, 1.06 mmol, 1.20 eq), HOBt (59.6 mg, 441 μmol, 0.500 eq) and DIEA (228 mg, 1.76 mmol, 307 μL, 2.00 eq) at 25 °C. The mixture was stirred at 25 °C for 1 h. LCMS showed the desired mass was detected. The organic layers were filtered and the filtrate was concentrated under reduced pressure to give a residue. The residue was purified by prep-HPLC (column: Phenomenex luna C18 100*40 mm*3 um; mobile phase: [H₂O (0.2% FA)-ACN]; gradient: 1%-30% B over 8.0 min) to give the title compound (Compound 242) (55.5 mg, 146 μmol, 16.6% yield, 98.5% purity) as a yellow solid. LCMS (ES⁺): m/z 374.2 [M+H]⁺.

### Example 134. Synthesis of cis-3-((4-(3-(1-acetylpiperidin-3-yl)phenyl)-5-fluoropyrimidin-2-yl)amino)cyclohexane-1-carboxamide (Compound 178)

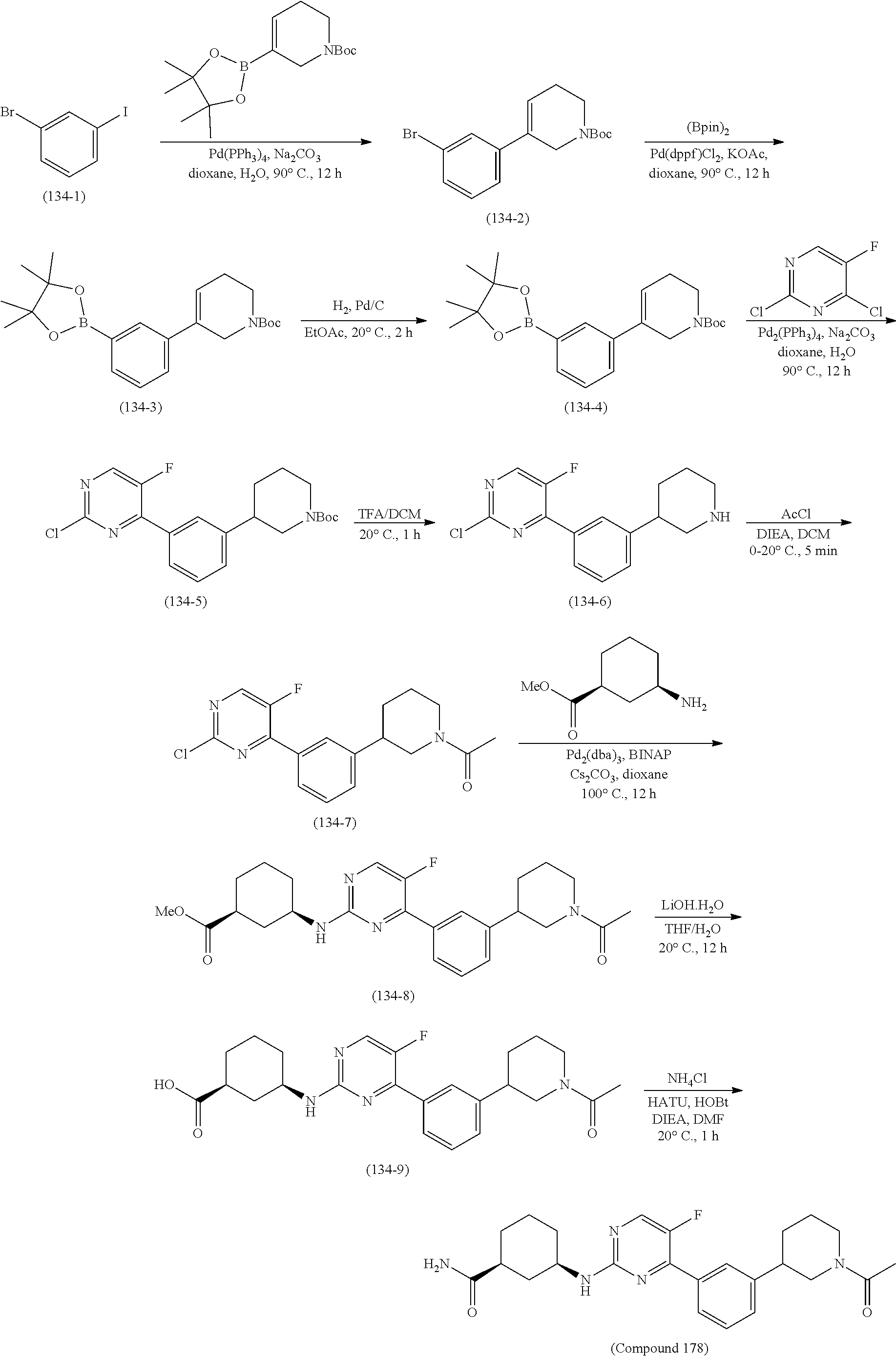

Synthesis of tert-butyl 5-(3-bromophenyl)-3,6-dihydropyridine-1 (2H)-carboxylate. To a solution of 1-bromo-3-iodobenzene (134-1) (11.0 g, 38.8 mmol, 4.95 mL, 1.20 eq) in dioxane (20 mL) and H₂O (2 mL) were added Pd(PPh₃)₄ (747 mg, 647 μmol, 0.0200 eq), tert-butyl 5-(4,4,5,5-tetramethyl-1,3,2-dioxaborolan-2-yl)-3,6-dihydro-2H-pyridine-1-carboxylate (10.0 g, 32.3 mmol, 1.00 eq) and Na₂CO₃ (6.86 g, 64.7 mmol, 2.00 eq) at 20 °C under N₂ atmosphere. The mixture was stirred at 90 °C for 12 h under N₂. LCMS showed the desired mass was detected. Water (15 mL) was added to the mixture at 20 °C. Then the reaction mixture was extracted with EtOAc (12 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=10:1 to 5:1) to give the title compound (134-2) (7.46 g, 22.1 mmol, 68.2% yield) as a colorless oil. LCMS (ES⁺): m/z 282.1 [M+H−56]⁺.

Synthesis of tert-butyl 5-(3-(4,4,5,5-tetramethyl-1,3,2-dioxaborolan-2-yl)phenyl)-3,6-dihydropyridine-1 (2H)-carboxylate (134-3). To a solution of tert-butyl 5-(3-bromophenyl)-3,6-dihydropyridine-1 (2H)-carboxylate (134-2) (7.46 g, 22.1 mmol, 1.00 eq) in dioxane (140 mL) were added KOAc (4.33 g, 44.1 mmol, 2.00 eq), 4,4,5,5-tetramethyl-2-(4,4,5,5-tetramethyl-1,3,2-dioxaborolan-2-yl)-1,3,2-dioxaborolane (6.72 g, 26.5 mmol, 1.20 eq) and Pd(dppf)Cl₂ (807 mg, 1.10 mmol, 0.0500 eq) at 20 °C under N₂ atmosphere. The mixture was stirred at 90 °C for 12 h under N₂. LCMS showed the desired mass was detected. The reaction mixture was concentrated under reduced pressure to remove solvent to give a residue. The residue was purified by column chromatography (SiO₂, Petroleum ether:Ethyl acetate=10:1 to 5:1) to give the title compound (134-3) (9.50 g, 21.5 mmol, 97.3% yield, 87.0% purity) as a colorless oil. LCMS (ES⁺): m/z 330.2 [M+H−56]⁺.

Synthesis of tert-butyl 3-(3-(4,4,5,5-tetramethyl-1,3,2-dioxaborolan-2-yl)phenyl)piperidine-1-carboxylate (134-4). To a solution of tert-butyl 5-(3-(4,4,5,5-tetramethyl-1,3,2-dioxaborolan-2-yl)phenyl)-3,6-dihydropyridine-1 (2H)-carboxylate (134-3) (9.50 g, 24.7 mmol, 1.00 eq) in EtOAc (150 mL) was added 10% Pd/C (10.0 g, 50.0% purity) at 20 °C under H₂ (15 psi) atmosphere. The mixture was stirred at 20 °C for 2 h under H₂. LCMS showed tert-butyl 5-(3-(4,4,5,5-tetramethyl-1,3,2-dioxaborolan-2-yl)phenyl)-3,6-dihydropyridine-1 (2H)-carboxylate (134-3) was consumed completely and the desired mass was detected. The organic layers were filtered and the filtrate was concentrated under reduced pressure to give the title compound (134-4) (9.90 g, crude) as a yellow oil. LCMS (ES⁺): m/z 332.1 [M+H−56]⁺

Synthesis of tert-butyl 3-(3-(2-chloro-5-fluoropyrimidin-4-yl)phenyl)piperidine-1-carboxylate (134-5). To a solution of tert-butyl 3-(3-(4,4,5,5-tetramethyl-1,3,2-dioxaborolan-2-yl)phenyl)piperidine-1-carboxylate (134-4) (9.00 g, 23.2 mmol, 1.00 eq) in dioxane (140 mL) and H₂O (14 mL) were added 2,4-dichloro-5-fluoro-pyrimidine (3.88 g, 23.2 mmol, 1.00 eq), Na₂CO₃ (4.93 g, 46.5 mmol, 2.00 eq) and Pd(PPh₃)₄ (537 mg, 465 μmol, 0.0200 eq) at 20 °C under N₂ atmosphere. The mixture was stirred at 90 °C for 12 h under N₂. LCMS showed tert-butyl 3-(3-(4,4,5,5-tetramethyl-1,3,2-dioxaborolan-2-yl)phenyl)piperidine-1-carboxylate (134-4) was consumed completely and the desired mass was detected. Water (100 mL) was added to the mixture at 20 °C. Then the reaction mixture was extracted with EtOAc (80 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=5:1 to 3:1) to give the title compound (134-5) (3.80 g, 9.70 mmol, 41.7% yield) as a yellow oil. LCMS (ES⁺): m/z 336.2 [M+H−56]⁺

Synthesis of 2-chloro-5-fluoro-4-(3-(piperidin-3-yl)phenyl)pyrimidine (134-6). To a solution of tert-butyl 3-(3-(2-chloro-5-fluoropyrimidin-4-yl)phenyl)piperidine-1-carboxylate (134-5) (1.00 g, 2.55 mmol, 1.00 eq) in DCM (9 mL) was added TFA (3 mL) at 20 °C. The mixture was stirred at 20 °C for 1 h. LCMS showed tert-butyl 3-(3-(2-chloro-5-fluoropyrimidin-4-yl)phenyl)piperidine-1-carboxylate (134-5) was consumed completely and the desired mass was detected. The reaction mixture was concentrated under reduced pressure to remove the solvent to give the title compound (134-6) (1.04 g, crude, TFA) as a green oil. LCMS (ES⁺): m/z 292.1 [M+H]⁺.

Synthesis of 1-(3-(3-(2-chloro-5-fluoropyrimidin-4-yl)phenyl)piperidin-1-yl)ethan-1-one (134-7). To a solution of 2-chloro-5-fluoro-4-(3-(piperidin-3-yl)phenyl)pyrimidine (134-6) (1.00 g, 2.46 mmol, 1.00 eq, TFA) in DCM (10 mL) were added DIEA (956 mg, 7.39 mmol, 1.29 mL, 3.00 eq) and acetyl chloride (193 mg, 2.46 mmol, 175 μL, 1.00 eq) at 0 °C. The mixture was stirred at 20 °C for 5 min. LCMS showed 2-chloro-5-fluoro-4-(3-(piperidin-3-yl)phenyl)pyrimidine (134-6) was consumed completely and the desired mass was detected. The reaction mixture was concentrated under reduced pressure to remove solvent to give a residue. The residue was purified by column chromatography (SiO₂, Petroleum ether:Ethyl acetate=1:3 to 0:1) to give the title compound (134-7) (550 mg, 1.65 mmol, 66.9% yield) as a yellow oil. LCMS (ES⁺): m/z 334.1 [M+H]⁺.

Synthesis of cis-methyl 3-((4-(3-(1-acetylpiperidin-3-yl)phenyl)-5-fluoropyrimidin-2-yl)amino)cyclohexane-1-carboxylate (134-8). To a solution of 1-(3-(3-(2-chloro-5-fluoropyrimidin-4-yl)phenyl)piperidin-1-yl)ethan-1-one (134-7) (300 mg, 899 μmol, 1.00 eq) in dioxane (5 mL) were added BINAP (39.2 mg, 62.9 μmol, 0.0700 eq), Pd₂(dba)₃ (41.2 mg, 44.9 μmol, 0.0500 eq), Cs₂CO₃ (586 mg, 1.80 mmol, 2.00 eq) and cis-methyl 3-aminocyclohexane-1-carboxylate (141 mg, 899 μmol, 1.00 eq) at 20 °C under N₂ atmosphere. The mixture was stirred at 100 °C for 12 h under N₂. LCMS showed 1-(3-(3-(2-chloro-5-fluoropyrimidin-4-yl)phenyl)piperidin-1-yl)ethan-1-one (134-7) was consumed completely and the desired mass was detected. The reaction mixture was concentrated under reduced pressure to remove solvent to give a residue. The residue was purified by prep-TLC (SiO₂, Ethyl acetate:Methanol=10:1) to give the title compound (134-8) (230 mg, 506 μmol, 56.3% yield) as a yellow solid. LCMS (ES⁺): m/z 455.3 [M+H]⁺.

Synthesis of cis-3-((4-(3-(1-acetylpiperidin-3-yl)phenyl)-5-fluoropyrimidin-2-yl)amino)cyclohexane-1-carboxylic acid (134-9). To a solution of cis-methyl 3-((4-(3-(1-acetylpiperidin-3-yl)phenyl)-5-fluoropyrimidin-2-yl)amino)cyclohexane-1-carboxylate (134-8) (230 mg, 506 μmol, 1.00 eq) in THE (2 mL) and H₂O (1 mL) was added LiOH·H₂O (63.7 mg, 1.52 mmol, 3.00 eq) at 20 °C. The mixture was stirred at 20 °C for 12 h. LCMS showed cis-methyl 3-((4-(3-(1-acetylpiperidin-3-yl)phenyl)-5-fluoropyrimidin-2-yl)amino)cyclohexane-1-carboxylate (134-8) was consumed completely and the desired mass was detected. The reaction mixture was concentrated under reduced to remove THF. HCl (1M) was added dropwise to the remaining aqueous phase to adjust pH to 2. The above aqueous solution was extracted with EtOAc (2 mL*3). The organic layers were combined, dried over anhydrous Na₂SO₄, filtered and concentrated under reduced pressure to give the title compound (134-9) (235 mg, crude) as a yellow solid. LCMS (ES⁺): m/z 441.3 [M+H]⁺.

Synthesis of cis-3-((4-(3-(1-acetylpiperidin-3-yl)phenyl)-5-fluoropyrimidin-2-yl)amino)cyclohexane-1-carboxamide (Compound 178). To a solution of cis-3-((4-(3-(1-acetylpiperidin-3-yl)phenyl)-5-fluoropyrimidin-2-yl)amino)cyclohexane-1-carboxylic acid (134-9) (230 mg, 522 μmol, 1.00 eq) in DMF (3 mL) were added HATU (238 mg, 627 μmol, 1.20 eq), DIEA (337 mg, 2.61 mmol, 455 μL, 5.00 eq), NH₄Cl (112 mg, 2.09 mmol, 4.00 eq) and HOBt (35.3 mg, 261 μmol, 0.500 eq) at 20 °C. The mixture was stirred at 20 °C for 1 h. LCMS showed cis-3-((4-(3-(1-acetylpiperidin-3-yl)phenyl)-5-fluoropyrimidin-2-yl)amino)cyclohexane-1-carboxylic acid (134-9) was consumed completely and the desired mass was detected. The organic layers were filtered and the filtrate was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 20%-50% B over 8.0 min) to give the title compound (Compound 178) (67.6 mg, 152 μmol, 29.1% yield, 98.9% purity) as a white solid. LCMS (ES⁺): m/z 440.2 [M+H]⁺.

### Example 135. Synthesis of 1-(4-((4-(3-(1-acetylpiperidin-3-yl)phenyl)-5-fluoropyrimidin-2-yl)amino)piperidin-1-yl)ethan-1-one (Compound 179)

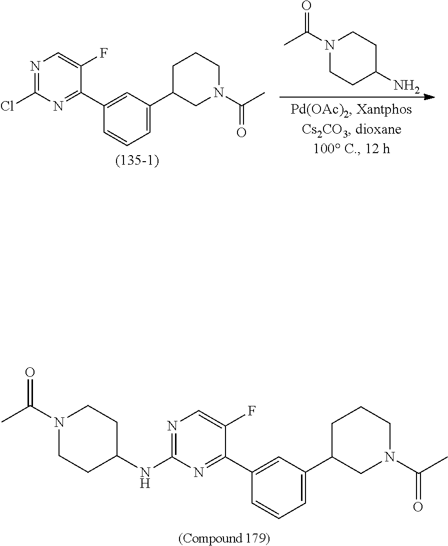

Synthesis of 1-(4-((4-(3-(1-acetylpiperidin-3-yl)phenyl)-5-fluoropyrimidin-2-yl)amino)piperidin-1-yl)ethan-1-one (Compound 179). To a solution of 1-(3-(3-(2-chloro-5-fluoropyrimidin-4-yl)phenyl)piperidin-1-yl)ethan-1-one (135-1) (500 mg, 1.50 mmol, 1.00 eq) in dioxane (7 mL) were added Pd(OAc)₂ (16.8 mg, 74.9 μmol, 0.05 eq), Xantphos (26.0 mg, 44.9 μmol, 0.0300 eq), Cs₂CO₃ (976 mg, 3.00 mmol, 2.00 eq) and 1-(4-amino-1-piperidyl)ethanone (213 mg, 1.50 mmol, 1.00 eq) at 20 °C under N₂ atmosphere. The mixture was stirred at 100 °C for 12 h under N₂. LCMS showed 1-(3-(3-(2-chloro-5-fluoropyrimidin-4-yl)phenyl)piperidin-1-yl)ethan-1-one (135-1) was consumed completely and the desired mass was detected. The reaction mixture was concentrated under reduced pressure to remove solvent. The residue was purified by prep-HPLC (column: Waters Xbridge Prep OBD C18 150*40 mm*10 um; mobile phase: [H₂O (10 mM NH₄HCO₃)-ACN]; gradient: 20%-50% B over 8.0 min) to give the title compound (Compound 179) (62.7 mg, 141 μmol, 9.38% yield, 98.5% purity) as a yellow solid. LCMS (ES⁺): m/z 440.2 [M+H]⁺.

### Example 136. Synthesis of trans-N-(4-((4-(3-(1-acetylpiperidin-3-yl)phenyl)-5-fluoropyrimidin-2-yl)amino)cyclohexyl)acetamide (Compound 174)

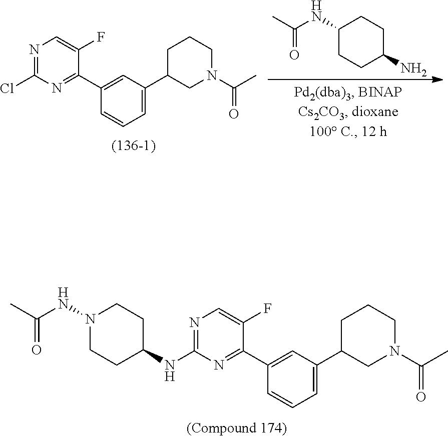

Synthesis of trans-N-(4-((4-(3-(1-acetylpiperidin-3-yl)phenyl)-5-fluoropyrimidin-2-yl)amino)cyclohexyl)acetamide (Compound 174). To a solution of 1-(3-(3-(2-chloro-5-fluoropyrimidin-4-yl)phenyl)piperidin-1-yl)ethan-1-one (136-1) (150 mg, 448 μmol, 1.00 eq) and trans-N-(4-aminocyclohexyl)acetamide (140 mg, 896 μmol, 2.00 eq) in dioxane (10 mL) were added BINAP (27.9 mg, 44.8 μmol, 0.100 eq), Cs₂CO₃ (438 mg, 1.34 mmol, 3.00 eq) and Pd₂(dba)₃ (41.0 mg, 44.8 μmol, 0.100 eq) at 25 °C under N₂. The mixture was stirred at 100 °C for 12 h under N₂. LCMS showed 1-(3-(3-(2-chloro-5-fluoropyrimidin-4-yl)phenyl)piperidin-1-yl)ethan-1-one (136-1) was consumed completely and the desired mass was detected. The mixture was diluted with H₂O (5 mL) and then extracted with EtOAc (5 mL*3). The combined organic layers were dried over Na₂SO₄, filtered and the filtrate was concentrated under reduced pressure to give a residue. The residue was purified by prep-HPLC (column: Phenomenex luna C18 100*40 mm*5 um; mobile phase: [H₂O (0.2% FA)-ACN]; gradient: 20%-60% B over 8.0 min) to give the title compound (Compound 174) (11.3 mg, 22.8 μmol, 5.08% yield, 91.4% purity) as a white solid. LCMS (ES⁺): m/z 454.2 [M+H]+.

### Example 137. Synthesis of cis-3-((5-fluoro-4-(3-(2-oxo-1,2-dihydropyridin-4-yl)phenyl)pyrimidin-2-yl)amino)cyclohexane-1-carboxamide (Compound 200)

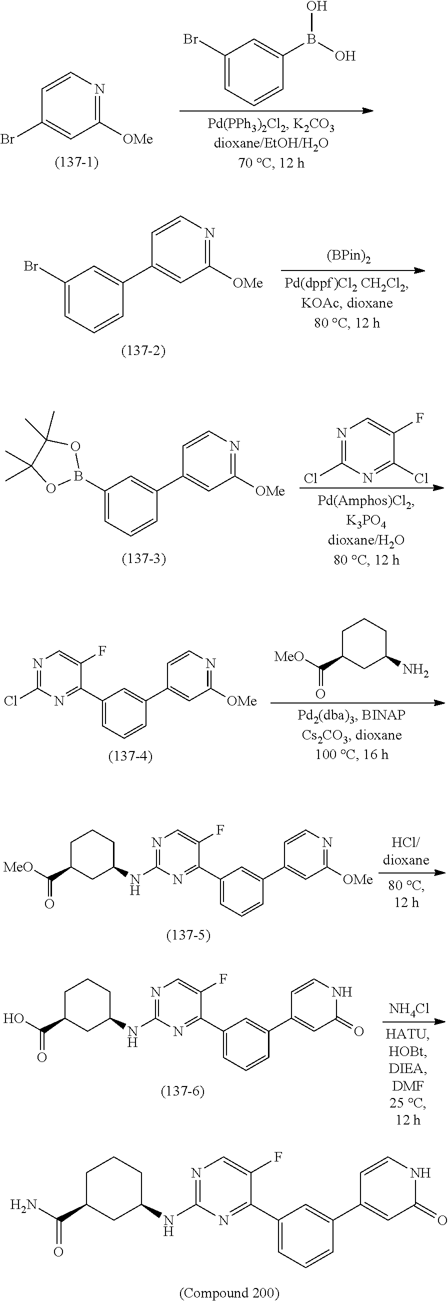

Synthesis of 4-(3-bromophenyl)-2-methoxypyridine (137-2). To a solution of 4-bromo-2-methoxypyridine (137-1) (5.00 g, 26.6 mmol, 1.00 eq) in dioxane (10 mL), H₂O (1 mL) and EtOH (2.5 mL) were added (3-bromophenyl)boronic acid (10.7 g, 53.2 mmol, 2.00 eq), K₂CO₃ (9.19 g, 66.5 mmol, 2.50 eq) and Pd(PPh₃)₂Cl₂ (933 mg, 1.33 mmol, 0.0500 eq) at 20 °C under N₂ atmosphere. The mixture was stirred at 70 °C for 12 h under N₂. LCMS showed the desired mass was detected. The reaction mixture was concentrated under reduced pressure to remove solvent to give a residue. The residue was purified by column chromatography (SiO₂, Petroleum ether:Ethyl acetate=50:1 to 10:1) to give the title compound (137-2) (4.40 g, 14.7 mmol, 27.6% yield, 88.0% purity) as a colorless oil. LCMS (ES⁺): m/z 264.0 [M+H]⁺.

Synthesis of 2-methoxy-4-(3-(4,4,5,5-tetramethyl-1,3,2-dioxaborolan-2-yl)phenyl)pyridine (137-3). To a solution of 4-(3-bromophenyl)-2-methoxypyridine (137-2) (4.40 g, 16.7 mmol, 1.00 eq) in dioxane (20 mL) were added KOAc (2.45 g, 25.0 mmol, 1.50 eq), Pd(dppf)Cl₂·CH₂Cl₂ (680 mg, 833 μmol, 0.0500 eq) and 4,4,5,5-tetramethyl-2-(4,4,5,5-tetramethyl-1,3,2-dioxaborolan-2-yl)-1,3,2-dioxaborolane (5.50 g, 21.7 mmol, 1.30 eq) at 25 °C under N₂ atmosphere. The mixture was stirred at 80 °C for 12 h under N₂. LCMS showed 4-(3-bromophenyl)-2-methoxypyridine (137-2) was consumed completely and the desired mass was detected. Water (50 mL) was added to the mixture at 25 °C. Then the reaction mixture was extracted with EtOAc (50 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=10:1 to 8:1) to give the title compound (137-3) (6.00 g, 16.3 mmol, 98.1% yield, 84.7% purity) as a colorless oil. LCMS (ES⁺): m/z 312.1 [M+H]⁺.

Synthesis of 2-chloro-5-fluoro-4-(3-(2-methoxypyridin-4-yl)phenyl)pyrimidine (137-4). To a solution of 2-methoxy-4-(3-(4,4,5,5-tetramethyl-1,3,2-dioxaborolan-2-yl)phenyl)pyridine (137-3) (6.00 g, 19.3 mmol, 1.00 eq) in dioxane (10 mL) and H₂O (0.1 mL) were added K₃PO₄ (8.19 g, 38.6 mmol, 2.00 eq), Pd(Amphos)Cl₂ (1.37 g, 1.93 mmol, 1.37 mL, 0.100 eq) and 2,4-dichloro-5-fluoro-pyrimidine (3.22 g, 19.3 mmol, 1.00 eq) at 25 °C under N₂ atmosphere. The mixture was stirred at 80 °C for 12 h under N₂. LCMS showed 2-methoxy-4-(3-(4,4,5,5-tetramethyl-1,3,2-dioxaborolan-2-yl)phenyl)pyridine (137-3) was consumed completely and the desired mass was detected. Water (60 mL) was added to the mixture at 25 °C. Then the reaction mixture was extracted with EtOAc (60 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=6:1 to 5:1) to give the title compound (137-4) (4.60 g, 12.9 mmol, 67.0% yield, 88.7% purity) as a yellow solid. LCMS (ES⁺): m/z 315.9 [M+H]⁺.

Synthesis of cis-methyl 3-((5-fluoro-4-(3-(2-methoxypyridin-4-yl)phenyl)pyrimidin-2-yl)amino)cyclohexane-1-carboxylate (137-5). To a solution of 2-chloro-5-fluoro-4-(3-(2-methoxypyridin-4-yl)phenyl)pyrimidine (137-4) (1.00 g, 3.17 mmol, 1.00 eq) in dioxane (10 mL) were added Cs₂CO₃ (2.06 g, 6.33 mmol, 2.00 eq), BINAP (138 mg, 222 μmol, 0.0700 eq), cis-methyl 3-aminocyclohexane-1-carboxylate (1.49 g, 9.50 mmol, 3.00 eq) and Pd₂(dba)₃ (145 mg, 158 μmol, 0.0500 eq) at 25 °C under N₂ atmosphere. The mixture was stirred at 100 °C for 12 h under N₂. LCMS showed 2-chloro-5-fluoro-4-(3-(2-methoxypyridin-4-yl)phenyl)pyrimidine (137-4) remained mostly and the desired mass was detected. The mixture was stirred at 100 °C for another 4 h. LCMS showed 2-chloro-5-fluoro-4-(3-(2-methoxypyridin-4-yl)phenyl)pyrimidine (137-4) remained little and the desired mass was detected. Water (15 mL) was added to the mixture at 25 °C. Then the reaction mixture was extracted with EtOAc (15 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=9:1 to 3:1) to give the title compound (137-5) (230 mg, 244 μmol, 7.70% yield, 46.3% purity) as a yellow oil. LCMS (ES⁺): m/z 437.1 [M+H]⁺.

Synthesis of cis-3-((5-fluoro-4-(3-(2-oxo-1,2-dihydropyridin-4-yl)phenyl)pyrimidin-2-yl)amino)cyclohexane-1-carboxylic acid (137-6). A solution of cis-methyl 3-((5-fluoro-4-(3-(2-methoxypyridin-4-yl)phenyl)pyrimidin-2-yl)amino)cyclohexane-1-carboxylate (137-5) (210 mg, 481 μmol, 1.00 eq) in HCl/dioxane (5 mL) was stirred at 80 °C for 12 h. LCMS showed cis-methyl 3-((5-fluoro-4-(3-(2-methoxypyridin-4-yl)phenyl)pyrimidin-2-yl)amino)cyclohexane-1-carboxylate (137-5) remained and the desired mass was detected. The reaction was concentrated under reduced pressure to give a residue. LiOH·H₂O (10%, in water) was added dropwise to the reaction mixture until the pH had reached 8. The above aqueous solution was washed with EtOAc (5 mL*3). Then HCl (1 M) was added dropwise to the aqueous phase to adjust pH to 3. Then it was extracted with EtOAc (5 mL*3). The organic layers were combined, dried over anhydrous Na₂SO₄, filtered and concentrated under reduced pressure to give the title compound (137-6) (100 mg, crude) as a yellow solid. LCMS (ES⁺): m/z 409.2 [M+H]⁺.

Synthesis of cis-3-((5-fluoro-4-(3-(2-oxo-1,2-dihydropyridin-4-yl)phenyl)pyrimidin-2-yl)amino)cyclohexane-1-carboxamide (Compound 200). To a solution of cis-3-((5-fluoro-4-(3-(2-oxo-1,2-dihydropyridin-4-yl)phenyl)pyrimidin-2-yl)amino)cyclohexane-1-carboxylic acid (137-6) (100 mg, 245 μmol, 1.00 eq) in DMF (3 mL) were added DIEA (31.6 mg, 245 μmol, 42.7 μL, 1.00 eq), HATU (186 mg, 490 μmol, 2.00 eq), HOBt (50.0 mg, 367 μmol, 1.50 eq) and NH₄Cl (65.5 mg, 1.22 mmol, 5.00 eq) at 25 °C. The mixture was stirred at 25 °C for 12 h. LCMS showed cis-3-((5-fluoro-4-(3-(2-oxo-1,2-dihydropyridin-4-yl)phenyl)pyrimidin-2-yl)amino)cyclohexane-1-carboxylic acid (137-6) was consumed completely and the desired mass was detected. The reaction mixture was concentrated under reduced pressure to get a residue, and the residue was purified by prep-HPLC (column: Waters Xbridge BEH C18 100*30 mm*10 um; mobile phase: [H₂O (10 mM NH₄HCO₃)-ACN]; gradient: 20%-50% B over 8.0 min) to give the title compound (Compound 200) (6.50 mg, 15.5 μmol, 7.01% yield, 97.1% purity) as a white solid. LCMS (ES⁺): m/z 408.1 [M+H]⁺.

### Example 138. Synthesis of 4-(3-(2-((1-acetylpiperidin-4-yl)amino)-5-fluoropyrimidin-4-yl)phenyl)pyridin-2(1H)-one (Compound 187)

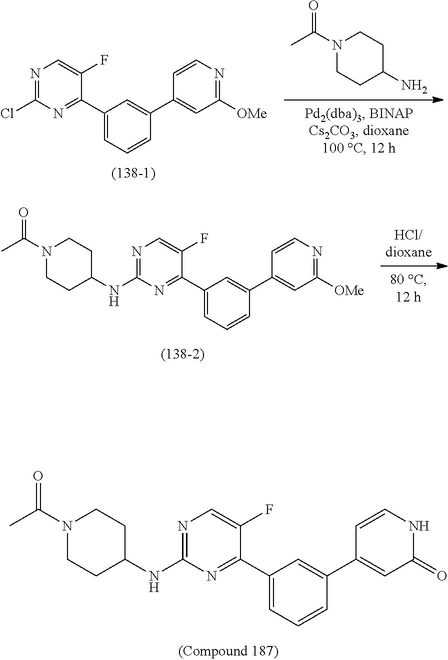

Synthesis of 1-(4-((5-fluoro-4-(3-(2-methoxypyridin-4-yl)phenyl)pyrimidin-2-yl)amino)piperidin-1-yl)ethan-1-one (138-2). To a solution of 2-chloro-5-fluoro-4-(3-(2-methoxypyridin-4-yl)phenyl)pyrimidine (138-1) (140 mg, 443 μmol, 1.00 eq) in dioxane (3 mL) were added 1-(4-amino-1-piperidyl)ethanone (63.1 mg, 443 μmol, 1.00 eq), Cs₂CO₃ (289 mg, 887 μmol, 2.00 eq), BINAP (19.3 mg, 31.0 μmol, 0.0700 eq) and Pd₂(dba)₃ (20.3 mg, 22.2 μmol, 0.0500 eq) at 25 °C under N₂ atmosphere. The mixture was stirred at 100 °C for 12 h under N₂. LCMS showed 2-chloro-5-fluoro-4-(3-(2-methoxypyridin-4-yl)phenyl)pyrimidine (138-1) was consumed completely and the desired mass was detected. Water (5 mL) was added to the mixture at 25 °C. Then the reaction mixture was extracted with EtOAc (5 mL*3). The organic layers were dried over anhydrous sodium sulfate, filtered and the filtrate was concentrated under reduced pressure to give a residue. The residue was purified by prep-TLC (SiO₂, Dichloromethane:Methanol=10:1) to give the title compound (138-2) (60.0 mg, 106 μmol, 23.9% yield, 74.4% purity) as a yellow solid. LCMS (ES⁺): m/z 422.1 [M+H]⁺.

Synthesis of 4-(3-(2-((1-acetylpiperidin-4-yl)amino)-5-fluoropyrimidin-4-yl)phenyl)pyridin-2(1H)-one (Compound 187). A solution of 1-(4-((5-fluoro-4-(3-(2-methoxypyridin-4-yl)phenyl)pyrimidin-2-yl)amino)piperidin-1-yl)ethan-1-one (138-2) (50.0 mg, 119 μmol, 1.00 eq) in HCl/dioxane (1 mL) was stirred at 80 °C for 12 h. LCMS showed 1-(4-((5-fluoro-4-(3-(2-methoxypyridin-4-yl)phenyl)pyrimidin-2-yl)amino)piperidin-1-yl)ethan-1-one (138-2) was consumed completely and the desired mass was detected. The reaction mixture was concentrated under reduced pressure to give a residue. The residue were purified by prep-HPLC (column: Waters Xbridge Prep OBD C18 150*40 mm*10 um; mobile phase: [H₂O (10 mM NH₄HCO₃)-ACN]; gradient: 15%-45% B over 8.0 min) to give the title compound (Compound 187) (8.90 mg, 21.8 μmol, 18.4% yield, 100% purity) as a white solid. LCMS (ES⁺): m/z 408.1 [M+H]⁺.

### Example 139. Synthesis of trans-N-(4-((5-fluoro-4-(3-(2-oxo-1,2-dihydropyridin-4-yl)phenyl)pyrimidin-2-yl)amino)cyclohexyl)acetamide (Compound 188)

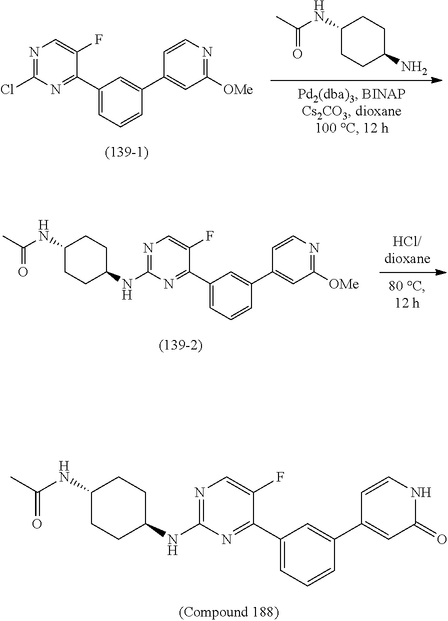 [SMILES: COc1cc(-c2cccc(-c3nc(Cl)ncc3F)c2)ccn1 ; CC(=O)N[C@H]1CC[C@H](N)CC1 ; COc1cc(-c2cccc(-c3nc(N[C@H]4CC[C@H](NC(C)=O)CC4)ncc3F)c2)ccn1 ; CC(=O)N[C@H]1CC[C@H](Nc2ncc(F)c(-c3cccc(-c4cc[nH]c(=O)c4)c3)n2)CC1]

Synthesis of trans-N-(4-((5-fluoro-4-(3-(2-methoxypyridin-4-yl)phenyl)pyrimidin-2-yl)amino)cyclohexyl)acetamide (139-2). To a solution of 2-chloro-5-fluoro-4-(3-(2-methoxypyridin-4-yl)phenyl)pyrimidine (139-1) (240 mg, 760 μmol, 1.00 eq) in dioxane (6 mL) were added trans-N-(4-aminocyclohexyl)acetamide (297 mg, 1.90 mmol, 2.50 eq), Cs₂CO₃ (495 mg, 1.52 mmol, 2.00 eq), BINAP (33.1 mg, 53.2 μmol, 0.0700 eq) and Pd₂(dba)₃ (34.8 mg, 38.0 μmol, 0.0500 eq) at 25 °C under N₂ atmosphere. The mixture was stirred at 100 °C for 12 h under N₂. LCMS showed 2-chloro-5-fluoro-4-(3-(2-methoxypyridin-4-yl)phenyl)pyrimidine (139-1) was consumed completely and the desired mass was detected. Water (10 mL) was added to the mixture at 25 °C. Then the reaction mixture was extracted with EtOAc (5 mL*3). The combined organic layers were dried over anhydrous sodium sulfate, filtered and the filtrate was concentrated under reduced pressure to give a residue. The residue was purified by prep-TLC (SiO₂, Dichloromethane:Methanol=10:1) to give the title compound (139-2) (60.0 mg, 95.6 μmol, 12.6% yield, 69.4% purity) as a yellow solid. LCMS (ES⁺): m/z 436.1 [M+H]⁺.

Synthesis of trans-N-(4-((5-fluoro-4-(3-(2-oxo-1,2-dihydropyridin-4-yl)phenyl)pyrimidin-2-yl)amino)cyclohexyl)acetamide (Compound 188). A solution of trans-N-(4-((5-fluoro-4-(3-(2-methoxypyridin-4-yl)phenyl)pyrimidin-2-yl)amino)cyclohexyl)acetamide (139-2) (50.0 mg, 115 μmol, 1.00 eq) in HCl/dioxane (1 mL) was stirred at 80 °C for 12 h. LCMS showed that trans-N-(4-((5-fluoro-4-(3-(2-methoxypyridin-4-yl)phenyl)pyrimidin-2-yl)amino)cyclohexyl)acetamide (139-2) was consumed completely and the desired mass was detected. The reaction mixture was concentrated under reduced pressure to give a residue. The residue were purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min) to give the title compound (Compound 188) (15.1 mg, 35.8 μmol, 31.2% yield, 100% purity) as a white solid. LCMS (ES⁺): m/z 422.1 [M+H]⁺.

### Example 140. Synthesis of trans-4-((5-fluoro-4-(3-(2-oxo-1,2-dihydropyridin-4-yl)phenyl)pyrimidin-2-yl)amino)cyclohexane-1-carboxamide (Compound 195)

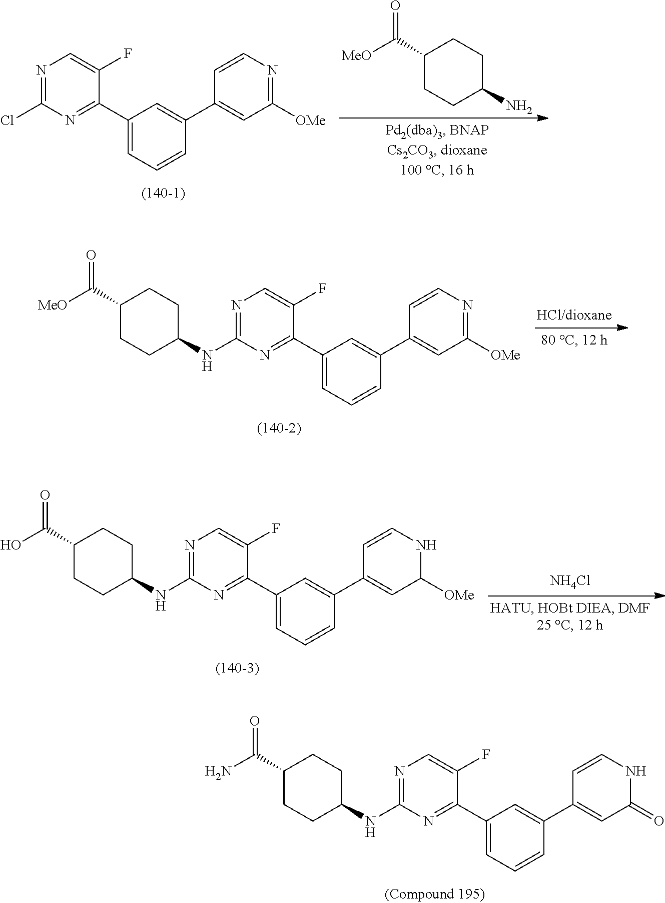

Synthesis of trans-methyl-4-((5-fluoro-4-(3-(2-methoxypyridin-4-yl)phenyl)pyrimidin-2-yl)amino)cyclohexane-1-carboxylate (140-2). To a solution of 2-chloro-5-fluoro-4-(3-(2-methoxypyridin-4-yl)phenyl)pyrimidine (140-1) (1.00 g, 3.17 mmol, 1.00 eq) in dioxane (10 mL) were added Cs₂CO₃ (2.06 g, 6.33 mmol, 2.00 eq), BINAP (138 mg, 222 μmol, 0.0700 eq), trans-methyl-4-aminocyclohexane-1-carboxylate (498 mg, 3.17 mmol, 1.00 eq) and Pd₂(dba)₃ (145 mg, 158 μmol, 0.0500 eq) at 25 °C under N₂ atmosphere. The mixture was stirred at 100 °C for 12 h under N₂. LCMS showed 2-chloro-5-fluoro-4-(3-(2-methoxypyridin-4-yl)phenyl)pyrimidine (140-1) remained and the desired mass was detected. The mixture was stirred a 100 °C for another 4 h. LCMS showed 2-chloro-5-fluoro-4-(3-(2-methoxypyridin-4-yl)phenyl)pyrimidine (140-1) remained little and the desired mass was detected. Water (20 mL) was added to the mixture at 25 °C. Then the reaction mixture was extracted with EtOAc (15 mL*3). The combined organic layers were dried over anhydrous sodium sulfate, filtered and the filtrate was concentrated under reduced pressure to give a residue. The residue was purified by column chromatography (SiO₂, Petroleum ether:Ethyl acetate=3:1 to 1:1) to give the title compound (140-2) (1.60 g, 2.07 mmol, 65.5% yield, 56.6% purity) as a red solid. LCMS (ES⁺): m/z 437.1 [M+H]⁺.

Synthesis of trans-4-((5-fluoro-4-(3-(2-oxo-1,2-dihydropyridin-4-yl)phenyl)pyrimidin-2-yl)amino)cyclohexane-1-carboxylic acid (140-3). A solution of trans-methyl-4-((5-fluoro-4-(3-(2-methoxypyridin-4-yl)phenyl)pyrimidin-2-yl)amino)cyclohexane-1-carboxylate (140-2) (500 mg, 1.15 mmol, 1.00 eq) in HCl/dioxane (5 mL) was stirred at 80 °C for 12 h. LCMS showed trans-methyl-4-((5-fluoro-4-(3-(2-methoxypyridin-4-yl)phenyl)pyrimidin-2-yl)amino)cyclohexane-1-carboxylate (140-2) was consumed completely and the desired mass was detected. LiOH·H₂O (10%) was added dropwise until the pH had reached 8. The aqueous solution was washed with EtOAc (5 mL). Then HCl (1 M) was added dropwise to the aqueous phase to adjust pH to 3. Then it was extracted with EtOAc (5 mL*3). The organic layers were combined, dried over anhydrous Na₂SO₄, filtered and concentrated under reduced pressure to give a residue to give the title compound (140-3) (140 mg, crude) as a yellow solid. LCMS (ES⁺): m/z 409.1 [M+H]⁺.

Synthesis of trans-4-((5-fluoro-4-(3-(2-oxo-1,2-dihydropyridin-4-yl)phenyl)pyrimidin-2-yl)amino)cyclohexane-1-carboxamide (Compound 195). To a solution of trans-4-((5-fluoro-4-(3-(2-oxo-1,2-dihydropyridin-4-yl)phenyl)pyrimidin-2-yl)amino)cyclohexane-1-carboxylic acid (140-3) (140 mg, 206 μmol, 1.00 eq) in DMF (3 mL) were added DIEA (26.6 mg, 206 μmol, 35.8 μL, 1.00 eq), HATU (156 mg, 411 μmol, 2.00 eq), HOBt (41.7 mg, 309 μmol, 1.50 eq) and NH₄Cl (55.0 mg, 1.03 mmol, 5.00 eq) at 25 °C. The mixture was stirred at 25 °C for 12 h. LCMS showed trans-4-((5-fluoro-4-(3-(2-oxo-1,2-dihydropyridin-4-yl)phenyl)pyrimidin-2-yl)amino)cyclohexane-1-carboxylic acid (140-3) was consumed completely and the desired mass was detected. The reaction mixture was filtered and the filtrate was concentrated under reduced pressure to give a residue. The residue were purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min) to give the title compound (Compound 195) (12.6 mg, 30.9 μmol, 15.0% yield, 100% purity) as a white solid. LCMS (ES⁺): m/z 408.1 [M+H]⁺.

### Example 141. Synthesis of 1-(4-((5-fluoro-4-morpholinopyrimidin-2-yl)amino)piperidin-1-yl)ethan-1-one (Compound 205)

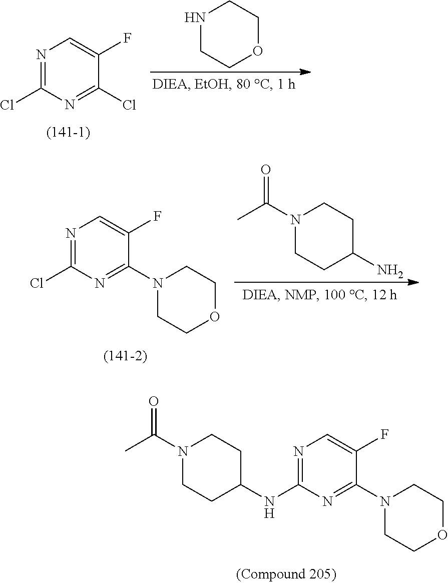

Synthesis of 4-(2-chloro-5-fluoropyrimidin-4-yl)morpholine (141-2). To a solution of 2,4-dichloro-5-fluoropyrimidine (141-1) (500 mg, 2.99 mmol, 1.00 eq) and morpholine (261 mg, 2.99 mmol, 264 μL, 1.00 eq) in EtOH (5 mL) was added DIEA (1.16 g, 8.98 mmol, 1.56 mL, 3.00 eq) at 15 °C. The mixture was stirred at 80 °C for 1 h. TLC (SiO₂, PE/EtOAc=3/1) indicated that 2,4-dichloro-5-fluoropyrimidine (141-1) was consumed completely and one new spot formed. The mixture was concentrated under reduced pressure to give a residue. The residue was purified by prep-TLC (SiO₂, PE/EtOAc=3/1) to give title compound (141-2) (480 mg, 2.21 mmol, 73.7% yield) as a white solid. LCMS (ES⁺): m/z 218.1 [M+H]⁺.

Synthesis of 1-(4-((5-fluoro-4-morpholinopyrimidin-2-yl)amino)piperidin-1-yl)ethan-1-one (Compound 205). To a solution of 4-(2-chloro-5-fluoropyrimidin-4-yl)morpholine (141-2) (280 mg, 1.29 mmol, 1.00 eq) and 1-(4-aminopiperidin-1-yl)ethan-1-one (366 mg, 2.57 mmol, 2.00 eq) in NMP (3 mL) was added DIEA (333 mg, 2.57 mmol, 448 μL, 2.00 eq) at 15 °C. The mixture was stirred at 100 °C for 12 h. LC-MS showed that 4-(2-chloro-5-fluoropyrimidin-4-yl)morpholine (141-2) was consumed completely and one main peak with desired mass was detected. The mixture was diluted with H₂O (5 mL) and extracted with EtOAc (2 mL*3). The combined organic layers were washed with brine (10 mL) dried over Na₂SO₄, filtered and the filtrate was concentrated under reduced pressure. The residue was purified by prep-HPLC (column: Waters Xbridge Prep OBD C18 150*40 mm*10 um; mobile phase: [H₂O (10 mM NH₄HCO₃)-ACN]; gradient: 20%-40% B over 8.0 min) to give title compound (Compound 205) (19.6 mg, 58.1 μmol, 4.51% yield, 95.8% purity) as a white solid. LCMS (ES⁺): m/z 324.1[M+H]⁺.

### Example 142. Synthesis of trans-N-(4-((5-fluoro-4-morpholinopyrimidin-2-yl)amino)cyclohexyl)acetamide (Compound 182)

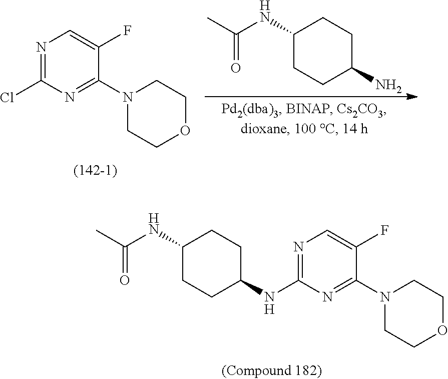 [SMILES: Fc1cnc(Cl)nc1N1CCOCC1 ; CC(=O)N[C@H]1CC[C@H](N)CC1 ; CC(=O)N[C@H]1CC[C@H](Nc2ncc(F)c(N3CCOCC3)n2)CC1]

Synthesis of trans-N-(4-((5-fluoro-4-morpholinopyrimidin-2-yl)amino)cyclohexyl)acetamide (Compound 182). To a solution of 4-(2-chloro-5-fluoropyrimidin-4-yl)morpholine (142-1) (300 mg, 1.38 mmol, 1.00 eq) and trans-N-(4-aminocyclohexyl)acetamide (215 mg, 1.38 mmol, 1.00 eq) in dioxane (6 mL) were added Pd₂(dba)₃ (126 mg, 138 μmol, 0.100 eq), BINAP (85.8 mg, 138 μmol, 0.100 eq) and Cs₂CO₃ (1.35 g, 4.14 mmol, 3.00 eq) at 25 °C under N₂. The mixture was stirred at 100 °C for 14 h under N₂. LC-MS showed that 4-(2-chloro-5-fluoropyrimidin-4-yl)morpholine the (142-1) was consumed completely the desired mass was detected. The mixture was concentrated in reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 20%-50% B over 8.0 min) to give the title compound (Compound 182) (23.7 mg, 68.8 μmol, 4.99% yield, 98.00% purity) as a white solid. LCMS (ES⁺): m/z 338.2 [M+H]⁺.

### Example 143. Synthesis of 1-(1-(2-((1-acetylpiperidin-4-yl)amino)-5-fluoropyrimidin-4-yl)piperidin-3-yl)pyridin-2(1H)-one (Compound 189)

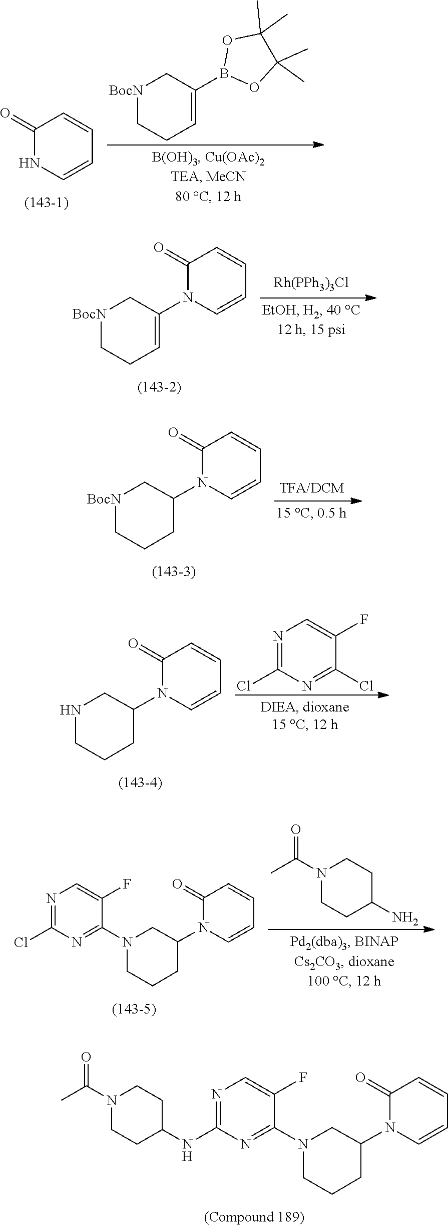

Synthesis of tert-butyl 2-oxo-5′,6′-dihydro-2H-[1,3′-bipyridine]-1′(2′H)-carboxylate (143-2). To a solution of tert-butyl 5-(4,4,5,5-tetramethyl-1,3,2-dioxaborolan-2-yl)-3,6-dihydropyridine-1 (2H)-carboxylate (16.7 g, 52.6 mmol, 1.00 eq) and pyridin-2(1H)-one (143-1) (5.00 g, 52.6 mmol, 1.00 eq) in MeCN (300 mL) were added boric acid (6.50 g, 105 mmol, 2.00 eq), Cu(OAc)₂ (9.55 g, 52.6 mmol, 1.00 eq) and TEA (16.0 g, 158 mmol, 22.0 mL, 3.00 eq) at 15 °C. The mixture was stirred at 80 °C for 12 h. LCMS showed the desired mass was detected. The mixture was diluted with H₂O (50 mL) and then extracted with EtOAc (50 mL*3). The combined organic layers were dried over Na₂SO₄, filtered and the filtrate was concentrated under reduced pressure to give a residue. The residue was purified by MPLC (SiO₂, PE/EtOAc 10/1 to 0/1) to give the title compound (143-2) (5.00 g, 18.1 mmol, 34.4% yield) as a yellow solid. LCMS (ES+): m/z 277.1 [M+H]+

Synthesis of tert-butyl 3-(2-oxopyridin-1 (2H)-yl)piperidine-1-carboxylate (143-3). To a stirred solution of tert-butyl 2-oxo-5′,6′-dihydro-2H-[1,3′-bipyridine]-1′(2′H)-carboxylate (143-2) (1.50 g, 5.43 mmol, 1.00 eq) in EtOH (30 mL) was added Rh(PPh₃)₃Cl (1.00 g, 1.09 mmol, 0.200 eq) at 15 °C. The resulting mixture was stirred at 40 °C for 12 h under H₂ (15 psi). LCMS showed tert-butyl 2-oxo-5′,6′-dihydro-2H-[1,3′-bipyridine]-1′(2′H)-carboxylate (143-2) was consumed completely and the desired mass was detected. The mixture was filtered and the filtrate was concentrated under reduced pressure to give a residue. The residue was purified by MPLC (SiO₂, PE/etOAc=10/1 to 0/1) to give the title compound (143-3) (1.20 g, 4.31 mmol, 79.4% yield) as a yellow solid. LCMS (ES+): m/z 279.1 [M+H]⁺

Synthesis of 1-(piperidin-3-yl)pyridin-2(1H)-one (143-4). Two batches in parallel were setup. A solution of tert-butyl 3-(2-oxopyridin-1 (2H)-yl)piperidine-1-carboxylate (143-3) (600 mg, 2.16 mmol, 1.00 eq) in DCM (8 mL) and TFA (4 mL) was stirred at 15 °C for 0.5 h. LCMS showed tert-butyl 3-(2-oxopyridin-1 (2H)-yl)piperidine-1-carboxylate (143-3) was consumed completely and the desired mass was detected. Two batches in parallel were combined and the mixture was concentrated under reduced pressure to give the title compound (143-4) (1.20 g, crude, TFA) as a yellow oil. LCMS (ES+): m/z 179.1 [M+H]⁺.

Synthesis of 1-(1-(2-chloro-5-fluoropyrimidin-4-yl)piperidin-3-yl)pyridin-2(1H)-one (143-5). To a solution of 1-(piperidin-3-yl)pyridin-2(1H)-one (143-4) (600 mg, 2.05 mmol, 1.00 eq, TFA) and DIEA (796 mg, 6.16 mmol, 1.07 mL, 3.00 eq) in dioxane (5 mL) was added 2,4-dichloro-5-fluoropyrimidine (343 mg, 2.05 mmol, 1.00 eq) at 15 °C. The resulting mixture was stirred at 15 °C for 12 h. LCMS showed 1-(piperidin-3-yl)pyridin-2 (1H)-one (143-4) was consumed completely and the desired mass was detected. The mixture was diluted with H₂O (10 mL) and then extracted with EtOAc (5 mL*3). The combined organic layers were dried over Na₂SO₄, filtered and the filtrate was concentrated under reduced pressure to give a residue. The residue was purified by prep-TLC (SiO₂, PE/EtOAc 0/1 to give the title compound (143-5) (400 mg, 1.30 mmol, 63.11% yield) as a yellow solid. LCMS (ES+): m/z 309.1 [M+H]⁺.

Synthesis of 1-(1-(2-((1-acetylpiperidin-4-yl)amino)-5-fluoropyrimidin-4-yl)piperidin-3-yl)pyridin-2(1H)-one (Compound 189). To a stirred solution of 1-(1-(2-chloro-5-fluoropyrimidin-4-yl)piperidin-3-yl)pyridin-2(1H)-one (143-5) (350 mg, 1.13 mmol, 1.00 eq) and 1-(4-aminopiperidin-1-yl)ethan-1-one (193 mg, 1.36 mmol, 1.20 eq) in dioxane (5 mL) were added Cs₂CO₃ (739 mg, 2.27 mmol, 2.00 eq), BINAP (42.4 mg, 68.0 μmol, 0.0600 eq) and Pd₂(dba)₃ (31.1 mg, 34.0 μmol, 0.0300 eq) at 15 °C. The resulting mixture was stirred at 100 °C for 12 h under N₂. LCMS showed 1-(1-(2-chloro-5-fluoropyrimidin-4-yl)piperidin-3-yl)pyridin-2(1H)-one (143-5) was consumed completely and the desired mass was detected. The mixture was diluted with H₂O (10 mL) and then extracted with EtOAc (5 mL*3). The combined organic layers were dried over Na₂SO₄, filtered and the filtrate was concentrated under reduced pressure to give a residue. The residue was purified by prep-HPLC (column: Phenomenex Luna C18 100*30 mm*3 um; mobile phase: [H₂O (0.2% FA)-ACN]; gradient: 1%-35% B over 8.0 min) to give the title compound (Compound 189) (99.4 mg, 214 μmol, 18.9% yield, 99.2% purity, FA) as a white solid. LCMS (ES+): m/z 415.2 [M+H]⁺.

### Example 144. Synthesis of 3-[1-[2-[(1-acetyl-4-piperidyl)amino]-5-fluoro-pyrimidin-4-yl]-3-piperidyl]-1H-pyridin-2-one (Compound 196)

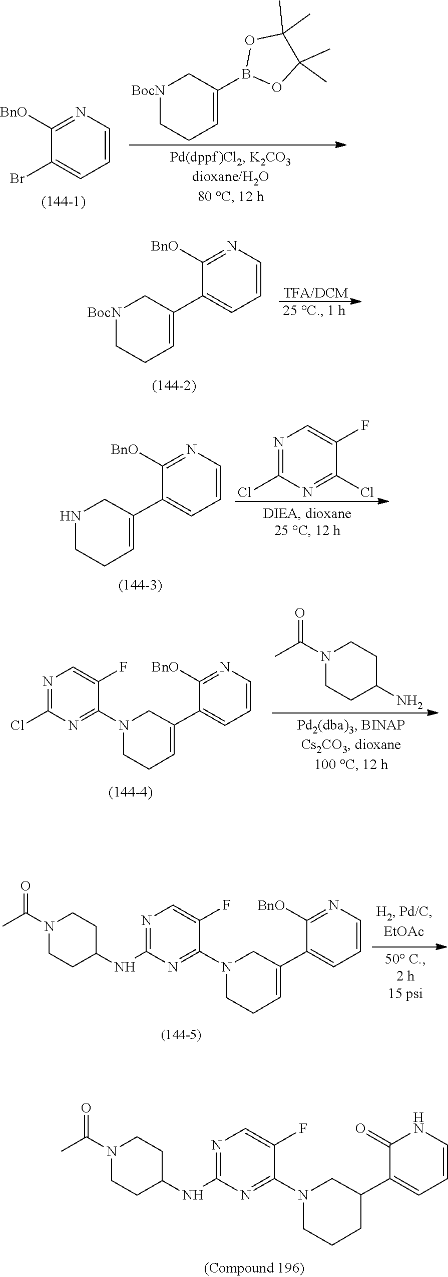

Synthesis of tert-butyl 5-(2-benzyloxy-3-pyridyl)-3,6-dihydro-2H-pyridine-1-carboxylate (144-2). To a solution of 2-benzyloxy-3-bromo-pyridine (144-1) (2.81 g, 9.09 mmol, 1.20 eq) and tert-butyl 5-(4,4,5,5-tetramethyl-1,3,2-dioxaborolan-2-yl)-3,6-dihydro-2H-pyridine-1-carboxylate (2.00 g, 7.57 mmol, 1.00 eq) in dioxane (40 mL) and H₂O (10 mL) were added K₂CO₃ (2.09 g, 15.1 mmol, 2.00 eq) and Pd(dppf)Cl₂ (554 mg, 757 μmol, 0.100 eq) at 25 °C under N₂, the mixture was stirred at 80 °C for 12 h under N₂. LC-MS showed that the desired mass was detected. To the mixture was added H₂O (50 mL) and the mixture was extracted with EtOAc (30 mL*3). The combined organic layers were washed with brine (30 mL*3), dried over Na₂SO₄, filtered and the filtrate was concentrated under reduced pressure to give the crude product. The crude product was purified by MPLC (SiO₂, Petroleum ether:Ethyl acetate=20:1 to 1:1) to give the title compound (144-2) (2.75 g, 7.50 mmol, 99.1% yield) as yellow solid. LCMS (ES⁺): m/z 367.2 [M+H]⁺

Synthesis of 2-benzyloxy-3-(1,2,3,6-tetrahydropyridin-5-yl)pyridine (144-3). To a solution of tert-butyl 5-(2-benzyloxy-3-pyridyl)-3,6-dihydro-2H-pyridine-1-carboxylate (144-2) (2.75 g, 7.50 mmol, 1.00 eq) in DCM (20 mL) was added TFA (5 mL) at 25 °C, the mixture was stirred at 25 °C for 1 h. LC-MS showed that the desired mass was detected. The mixture was concentrated under reduced pressure to give the title compound (144-3) (2.85 g, crude, TFA) as yellow oil liquid. LCMS (ES⁺): m/z 267.1 [M+H]⁺.

Synthesis of 4-[5-(2-benzyloxy-3-pyridyl)-3,6-dihydro-2H-pyridin-1-yl]-2-chloro-5-fluoro-pyrimidine (144-4). To a solution of 2-benzyloxy-3-(1,2,3,6-tetrahydropyridin-5-yl)pyridine (144-3) (2.85 g, 7.49 mmol, 1.00 eq, TFA) and 2,4-dichloro-5-fluoro-pyrimidine (1.25 g, 7.49 mmol, 1.00 eq) in dioxane (30 mL) was added DIEA (3.48 g, 27.0 mmol, 4.71 mL, 3.6 eq) at 25 °C, the mixture was stirred at 25 °C for 12 h. LC-MS showed that the desired mass was detected. The mixture was added to H₂O (100 mL) and filtered. The filter cake was dried under reduced pressure to give the title compound (144-4) (2.42 g, crude) as yellow solid. LCMS (ES⁺): m/z 397.0 [M+H]⁺.

Synthesis of 1-[4-[[4-[5-(2-benzyloxy-3-pyridyl)-3,6-dihydro-2H-pyridin-1-yl]-5-fluoro-pyrimidin-2-yl]amino]-1-piperidyl]ethanone (144-5). To a solution of 2′-(benzyloxy)-1-(2-chloro-5-fluoropyrimidin-4-yl)-1,2,5,6-tetrahydro-3,3′-bipyridine (144-4) (2.25 g, 5.67 mmol, 1 eq) and 1-(4-amino-1-piperidyl)ethanone (967.47 mg, 6.80 mmol, 1.2 eq) in dioxane (30 mL) were added Cs₂CO₃ (3.69 g, 11.34 mmol, 2 eq), Pd₂(dba)₃ (778.78 mg, 850.46 μmol, 0.15 eq) and BINAP (706.08 mg, 1.13 mmol, 0.2 eq) at 25 °C under N₂, the mixture was stirred at 100 °C for 12 h under N₂. LC-MS showed that the desired mass was detected. To the mixture was added H₂O (30 mL) and the mixture was extracted with EtOAc (20 mL*3). The combined organic layers were washed with brine (10 mL*3), dried over Na₂SO₄, filtered and the filtrate was concentrated in vacuo to give the crude product. The mixture was purified by MPLC (SiO₂, Petroleum ether/Ethyl acetate=1/0 to 1/1) to give the title compound (144-5) (1.62 g, 3.22 mmol, 56.85% yield) as yellow solid. LCMS (ES⁺): m/z 503.2 [M+H]⁺.

Synthesis of 3-[1-[2-[(I-acetyl-4-piperidyl)amino]-5-fluoro-pyrimidin-4-yl]-3-piperidyl]-1H-pyridin-2-one (Compound 196). To a solution of 1-[4-[[4-[5-(2-benzyloxy-3-pyridyl)-3,6-dihydro-2H-pyridin-1-yl]-5-fluoro-pyrimidin-2-yl]amino]-1-piperidyl]ethanone (144-5) (500 mg, 995 μmol, 1.00 eq) in EtOAc (20 mL) was added 10% Pd/C (1 g, 50% purity) at 25 °C, the mixture was stirred at 50 °C for 2 h under H₂ (15 psi). LC-MS showed that the desired mass was detected. The mixture was filtered and the filtrate was concentrated under reduced pressure to give a residue. This residue was purified by prep-HPLC (FA) (column: Phenomenex luna C18 100*40 mm*5 um; mobile phase: [H₂O (0.2% FA)-ACN]; gradient: 15%-50% B over 8.0 min) to give the title compound (Compound 196) (39.2 mg, 92.6 μmol, 9.31% yield, 98.00% purity) as white solid. LCMS (ES⁺): m/z 415.2 [M+H]⁺.

### Example 145. Synthesis of 4-(1-(2-((1-acetylpiperidin-4-yl)amino)-5-fluoropyrimidin-4-yl)piperidin-3-yl)pyridin-2(1H)-one (Compound 224)

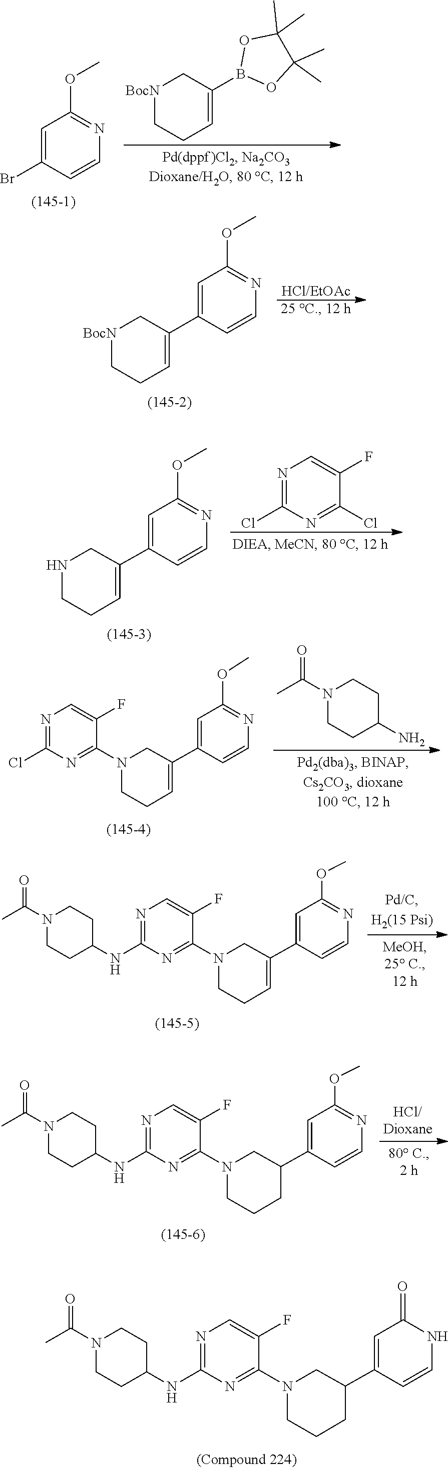

Synthesis of tert-butyl 2′-methoxy-5,6-dihydro-[3,4′-bipyridine]-1 (2H)-carboxylate (145-2). To a solution of 4-bromo-2-methoxy-pyridine (145-1) (4.00 g, 21.2 mmol, 1.00 eq) in dioxane (150 mL) and H₂O (40 mL) was added tert-butyl 5-(4,4,5,5-tetramethyl-1,3,2-dioxaborolan-2-yl)-3,6-dihydro-2H-pyridine-1-carboxylate (6.58 g, 21.2 mmol, 1.00 eq) and Pd(dppf)Cl₂ (1.56 g, 2.13 mmol, 0.100 eq) and Na₂CO₃ (6.76 g, 63.8 mmol, 3.00 eq) at 25 °C. The mixture was stirred at 80 °C for 12 h under N₂ atmosphere. LC-MS showed desired mass was detected. The reaction mixture was concentrated under reduced pressure. The residue was diluted with water 200 mL and extracted with Ethyl acetate (200 mL*2). The combined organic layers were washed with brine (200 mL*2), filtered and concentrated under reduced pressure. The residue was purified by column chromatography (SiO₂, Petroleum ether:Ethyl acetate=3:1 to 2:1) to give the title compound (145-2) (6.00 g, 17.7 mmol, 83.3% yield, 85.8% purity) as a brown oil. LCMS (ES⁺): m/z 291.2 [M+H]⁺.

Synthesis of 2′-methoxy-1,2,5,6-tetrahydro-3,4′-bipyridine (145-3). To a solution of tert-butyl 2′-methoxy-5,6-dihydro-[3,4′-bipyridine]-1 (2H)-carboxylate (145-2) (4.00 g, 13.7 mmol, 1.00 eq) was added HCl/EtOAc (4.00 M, 3.44 mL, 1.00 eq) at 25 °C. The mixture was stirred at 25 °C for 12 h. LC-MS showed the desired mass was detected. The reaction mixture was concentrated under reduced pressure to give the title compound (145-3) (3.60 g, crude, HCl) as a white solid. LCMS (ES⁺): m/z 191.2 [M+H]⁺.

Synthesis of 1-(2-chloro-5-fluoropyrimidin-4-yl)-2′-methoxy-1,2,5,6-tetrahydro-3,4′-bipyridine (145-4). To a solution of 2′-methoxy-1,2,5,6-tetrahydro-3,4′-bipyridine (145-3) (3.60 g, 15.8 mmol, 1.00 eq, HCl) in MeCN (20 mL) was added DIEA (6.16 g, 47.6 mmol, 8.30 mL, 3.00 eq) and 2,4-dichloro-5-fluoro-pyrimidine (2.65 g, 15.8 mmol, 1.00 eq) at 25 °C. The mixture was stirred at 80 °C for 12 h. LC-MS showed the desired mass was detected. The reaction diluted with H₂O 30 mL and combined organic layers were washed with EtOAc (50 mL*3), and concentrated under reduced pressure. The residue was purified by column chromatography (SiO₂, Petroleum ether:Ethyl acetate=1:0 to 1:1) to give the title compound (145-4) (3.50 g, 10.7 mmol, 67.9% yield, 98.8% purity) as a yellow solid. LCMS (ES⁺): m/z 321.1 [M+H]⁺.

Synthesis of 1-(4-((5-fluoro-4-(2′-methoxy-5,6-dihydro-[3,4′-bipyridin]-1 (2H)-yl)pyrimidin-2-yl)amino)piperidin-1-yl)ethan-1-one (145-5). A mixture of 1-(2-chloro-5-fluoropyrimidin-4-yl)-2′-methoxy-1,2,5,6-tetrahydro-3,4′-bipyridine (145-4) (2.00 g, 6.24 mmol, 1.00 eq), 1-(4-amino-1-piperidyl)ethanone (886 mg, 6.24 mmol, 1.00 eq), Pd₂(dba)₃ (1.14 g, 1.25 mmol, 0.200 eq), BINAP (776 mg, 1.25 mmol, 0.200 eq) and Cs₂CO₃ (6.09 g, 18.7 mmol, 3.00 eq) in dioxane (20 mL) at 25 °C was degassed and purged with N₂ for 3 times, and then the mixture was stirred at 100 °C for 12 h under N₂ atmosphere. LC-MS showed the desired mass was detected. The reaction diluted with H₂O 30 mL and combined organic layers were extracted with EtOAc (50 mL*3), and concentrated under reduced pressure. The residue was purified by column chromatography (SiO₂, Dichloromethane:Methanol=1:0 to 5:1) to give the title compound (145-5) (3.00 g, 5.49 mmol, 88.0% yield, 78.0% purity) as a white solid. LCMS (ES⁺): m/z 427.4 [M+H]⁺.

Synthesis of 1-(4-((5-fluoro-4-(3-(2-methoxypyridin-4-yl)piperidin-1-yl)pyrimidin-2-yl)amino)piperidin-1-yl)ethan-1-one (145-6). To a solution of 1-(4-((5-fluoro-4-(2′-methoxy-5,6-dihydro-[3,4′-bipyridin]-1 (2H)-yl)pyrimidin-2-yl)amino)piperidin-1-yl)ethan-1-one (145-5) (1.00 g, 2.34 mmol, 1.00 eq) in MeOH (25 mL) was added 10% Pd/C (499 mg, 2.34 mmol, 50.0% purity, 1.00 eq) at 25 °C under N₂ atmosphere. The suspension was degassed and purged with H₂ for 3 times. The mixture was stirred under H₂ (15 Psi) at 25 °C for 12 h. LC-MS showed the desired mass was detected. The reaction mixture was filtered and concentrated under reduced pressure to give the title compound (145-6) (600 mg, crude) as a brown oil. LCMS (ES⁺): m/z 429.2 [M+H]⁺.

Synthesis of 4-(1-(2-((1-acetylpiperidin-4-yl)amino)-5-fluoropyrimidin-4-yl)piperidin-3-yl)pyridin-2(1H)-one (Compound 224). To a solution of 1-(4-((5-fluoro-4-(3-(2-methoxypyridin-4-yl)piperidin-1-yl)pyrimidin-2-yl)amino)piperidin-1-yl)ethan-1-one (145-6) (300 mg, 700 μmol, 1.00 eq) in HCl/dioxane (15 mL) at 25 °C. The mixture was stirred at 80 °C for 2 h. LC-MS showed the desired mass was detected. The reaction was quenched by addition H₂O 5 mL at 0 °C. The residue was purified by prep-HPLC (column: Phenomenex Luna C18 100*30 mm*3 um; mobile phase: [H₂O (0.2% FA)-ACN]; gradient: 1%-35% B over 8.0 min) to give the title compound (Compound 224) (35.2 mg, 77.8 μmol, 11.1% yield, 98.9% purity, 0.71 FA) as a white solid. LCMS (ES⁺): m/z 415.2 [M+H]⁺.

### Example 146. Synthesis of 5-(1-(2-((1-acetylpiperidin-4-yl)amino)-5-fluoropyrimidin-4-yl)piperidin-3-yl)pyridin-2(1H)-one (Compound 199)

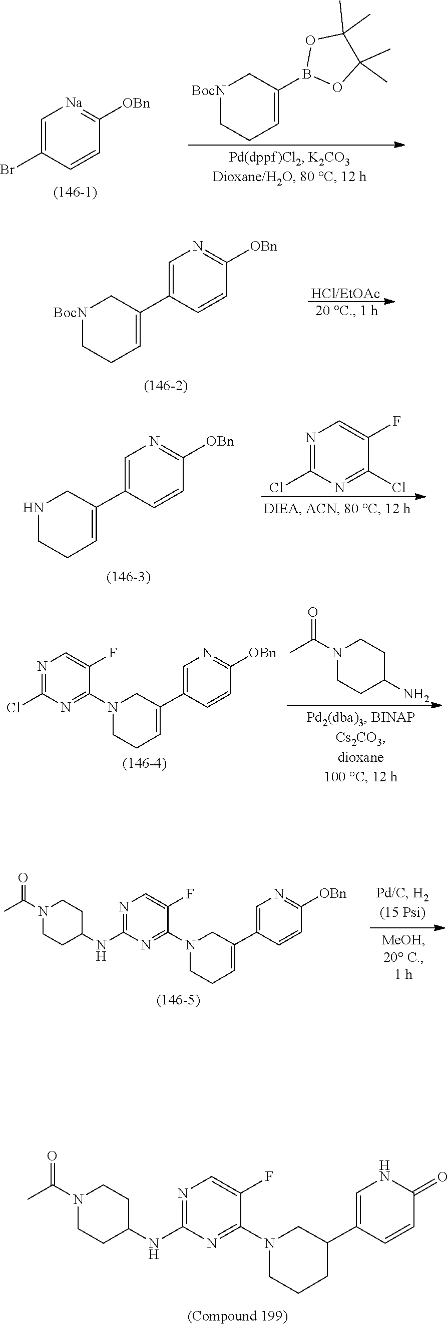

Synthesis of tert-butyl 6′-(benzyloxy)-5,6-dihydro-[3,3′-bipyridine]-1 (2H)-carboxylate (146-2). A mixture of 2-benzyloxy-5-bromo-pyridine (146-1) (1.69 g, 6.40 m mol, 1.10 eq), tert-butyl 5-(4,4,5,5-tetramethyl-1,3,2-dioxaborolan-2-yl)-3,6-dihydropyridine-1 (2H)-carboxylate (1.80 g, 5.82 mmol, 1.00 eq), Na₂CO₃ (1.85 g, 17.4 mmol, 3.00 eq) and Pd(dppf)Cl₂ (425 mg, 582 μmol, 0.100 eq) in dioxane (20 mL) and H₂O (5 mL) was degassed at 20 °C and purged with N₂ for 3 times, and then the mixture was stirred at 80 °C for 12 h under N₂ atmosphere. LC-MS showed the desired mass was detected. The reaction mixture was diluted with H₂O 10 mL and extracted with EtOAc 10 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (146-2) (1.60 g, 3.88 mmol, 66.6% yield, 88.9% purity) as a yellow solid. LCMS (ES⁺): m/z 367.2 [M+H]⁺.

Synthesis of 6′-(benzyloxy)-1,2,5,6-tetrahydro-3,3′-bipyridine (146-3). A mixture of tert-butyl 6′-(benzyloxy)-5,6-dihydro-[3,3′-bipyridine]-1 (2H)-carboxylate (146-2) (0.80 g, 2.18 mmol, 1.00 eq) in HCl/EtOAc (4.00 M, 10.0 mL, 18.3 eq) was stirred at 20 °C for 1 h. LC-MS showed the desired mass was detected. The reaction mixture was concentrated under reduced pressure to give the title compound (146-3) (700 mg, crude) as a yellow solid. LCMS (ES⁺): m/z 267.1 [M+H]⁺.

Synthesis of 6′-(benzyloxy)-1-(2-chloro-5-fluoropyrimidin-4-yl)-1,2,5,6-tetrahydro-3,3′-bipyridine (146-4). To a solution of 6′-(benzyloxy)-1,2,5,6-tetrahydro-3,3′-bipyridine (146-3) (600 mg, 2.25 mmol, 1.00 eq) and 2,4-dichloro-5-fluoro-pyrimidine (451 mg, 2.70 mmol, 1.20 eq) in MeCN (10 mL) was added DIEA (873 mg, 6.76 mmol, 1.18 mL, 3.00 eq) at 20 °C. The mixture was stirred at 80 °C for 12 h. LC-MS showed the desired mass was detected. The reaction mixture was quenched by addition H₂O 10 mL, and then extracted with EtOAc 30 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (146-4) (500 mg, 974 μmol, 43.25% yield, 77.34% purity) as a yellow solid. LCMS (ES⁺): m/z 397.1 [M+H]⁺.

Synthesis of 1-(4-((4-(6′-(benzyloxy)-5,6-dihydro-[3,3′-bipyridin]-1 (2H)-yl)-5-fluoropyrimidin-2-yl)amino)piperidin-1-yl)ethan-1-one (146-5). A mixture of 6′-(benzyloxy)-1-(2-chloro-5-fluoropyrimidin-4-yl)-1,2,5,6-tetrahydro-3,3′-bipyridine (146-4) (500 mg, 1.26 mmol, 1.00 eq), 1-(4-amino-1-piperidyl)ethanone (214 mg, 1.51 mmol, 1.20 eq), BINAP (78.4 mg, 125 μmol, 0.100 eq) and Cs₂CO₃ (821 mg, 2.52 mmol, 2.00 eq) Pd₂(dba)₃ (115 mg, 125 μmol, 0.100 eq) in dioxane (10 mL) at 20 °C was degassed and purged with N₂ for 3 times, and then the mixture was stirred at 100 °C for 12 h under N₂ atmosphere. LC-MS showed the desired mass was detected. The reaction mixture was diluted with H₂O 5 mL and extracted with EtOAc 5 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (146-5) (200 mg, 332 μmol, 26.4% yield, 83.5% purity) as a yellow solid. LCMS (ES⁺): m/z 503.2 [M+H]⁺.

Synthesis of 5-(1-(2-((1-acetylpiperidin-4-yl)amino)-5-fluoropyrimidin-4-yl)piperidin-3-yl)pyridin-2(1H)-one (Compound 199). To a solution of 1-(4-((4-(6′-(benzyloxy)-5,6-dihydro-[3,3′-bipyridin]-1 (2H)-yl)-5-fluoropyrimidin-2-yl)amino)piperidin-1-yl)ethan-1-one (146-5) (100 mg, 198 μmol, 1.00 eq) in MeOH (2 mL) was added 10% Pd/C (42.3 mg, 198 μmol, 50% purity, 1.00 eq) under N₂ atmosphere at 20 °C. The suspension was degassed and purged with H₂ for 3 times. The mixture was stirred under H₂ (15 Psi) at 20 °C for 1 h. LC-MS showed the desired mass was detected. The reaction mixture was filtered and concentrated under reduced pressure. The residue was purified by prep-HPLC (column: Phenomenex luna C18 100*40 mm*3 um; mobile phase: [H₂O (0.1% TFA)-ACN]; gradient: 1%-30% B over 8.0 min) to give the title compound (Compound 199) (35.6 mg, 85.67 μmol, 43.06% yield, 99.74% purity) as a white solid. LCMS (ES+): m/z 415.2 [M+H]+.

### Example 147. Synthesis of trans-(R)-1-(2-((4-acetamidocyclohexyl)amino)-5-fluoropyrimidin-4-yl)-N-cyclopropylpiperidine-3-carboxamide (Compound 197)

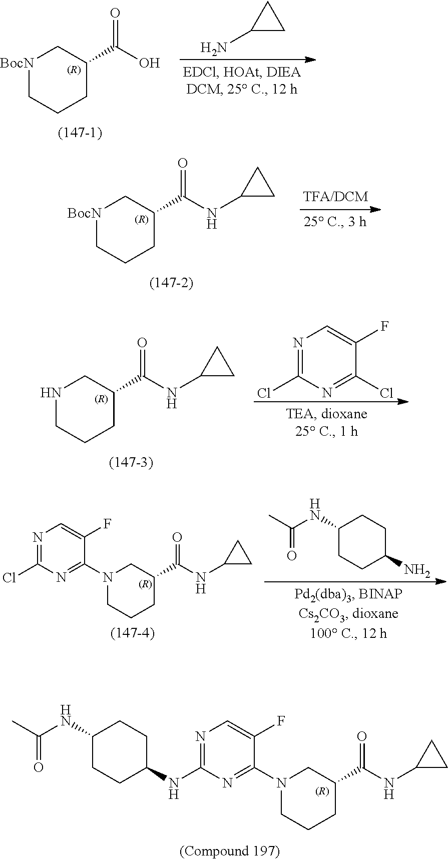

Synthesis of tert-butyl (R)-3-(cyclopropylcarbamoyl)piperidine-1-carboxylate (147-2). To a solution of (R)-1-(tert-butoxycarbonyl)piperidine-3-carboxylic acid (147-1) (1.00 g, 4.36 mmol, 1.00 eq) in DCM (10 mL) were added DIEA (1.78 g, 13.8 mmol, 2.40 mL, 3.16 eq), EDCI (1.78 g, 9.29 mmol, 2.13 eq), HOAt (950 mg, 6.98 mmol, 976 μL, 1.60 eq) and cyclopropanamine (249 mg, 4.36 mmol, 302 μL, 1.00 eq) at 0 °C. The mixture was stirred at 25 °C for 12 h. LCMS showed (R)-1-(tert-butoxycarbonyl)piperidine-3-carboxylic acid (147-1) remained and the desired mass was detected. The reaction mixture was concentrated under reduced pressure to remove solvent to give a residue. The residue was purified by MPLC (SiO₂, Petroleum ether:Ethyl acetate=1:0 to 0:1) (color developing reagent: iodine) to give the title compound (147-2) (600 mg, 1.77 mmol, 40.7% yield, 79.4% purity) as a white solid. LCMS (ES⁺): m/z 269.1 [M+H]⁺

Synthesis of (R)—N-cyclopropylpiperidine-3-carboxamide (147-3). To a solution of tert-butyl (R)-3-(cyclopropylcarbamoyl)piperidine-1-carboxylate (147-2) (550 mg, 2.05 mmol, 1.00 eq) in DCM (6 mL) was added TFA (2 mL) at 25 °C. The mixture was stirred at 25 °C for 3 h. LCMS showed tert-butyl (R)-3-(cyclopropylcarbamoyl)piperidine-1-carboxylate (147-2) was consumed completely and the desired mass was detected. The reaction mixture was concentrated under reduced pressure to remove solvent to give the title compound (147-3) (550 mg, crude, TFA) as a pink oil. LCMS (ES⁺): m/z 169.2 [M+H]⁺.

Synthesis of (R)-1-(2-chloro-5-fluoropyrimidin-4-yl)-N-cyclopropylpiperidine-3-carboxamide (147-4). To a solution of (R)—N-cyclopropylpiperidine-3-carboxamide (147-3) (550 mg, 1.95 mmol, 1.00 eq, TFA) in dioxane (10 mL) were added TEA (217 mg, 2.14 mmol, 298 μL, 1.10 eq) and 2,4-dichloro-5-fluoro-pyrimidine (325 mg, 1.95 mmol, 1.00 eq) at 25 °C. The mixture was stirred at 25 °C for 1 h. LCMS showed (R)—N-cyclopropylpiperidine-3-carboxamide (147-3) was consumed completely and the desired mass was detected. Water (10 mL) was added to the mixture at 25 °C. Then the reaction mixture was extracted with EtOAc (8 mL*3). The organic layers were dried over anhydrous sodium sulfate, filtered and the filtrate was concentrated under reduced pressure to give a residue. The residue was purified by prep-TLC (SiO₂, Petroleum ether:Ethyl acetate=0:1) to give the title compound (147-4) (560 mg, 1.29 mmol, 66.4% yield, 69.0% purity) as a white solid. LCMS (ES⁺): m/z 299.1 [M+H]⁺.

Synthesis of trans-(R)-1-(2-((4-acetamidocyclohexyl)amino)-5-fluoropyrimidin-4-yl)-N-cyclopropylpiperidine-3-carboxamide (Compound 197). To a solution of (R)-1-(2-chloro-5-fluoropyrimidin-4-yl)-N-cyclopropylpiperidine-3-carboxamide (147-4) (200 mg, 669 μmol, 1.00 eq) in dioxane (2 mL) were added Cs₂CO₃ (436 mg, 1.34 mmol, 2.00 eq), BINAP (29.2 mg, 46.9 μmol, 0.0700 eq), trans-N-(4-aminocyclohexyl)acetamide (261 mg, 1.67 mmol, 2.50 eq) and Pd₂(dba)₃ (30.7 mg, 33.5 μmol, 0.0500 eq) at 25 °C under N₂ atmosphere. The mixture was stirred at 100 °C for 12 h under N₂. LCMS showed (R)-1-(2-chloro-5-fluoropyrimidin-4-yl)-N-cyclopropylpiperidine-3-carboxamide (147-4) remained little and the desired mass was detected. The reaction mixture was filtered and the filtrate was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 15%-45% B over 8.0 min) to give the title compound (Compound 197) (13.4 mg, 31.5 μmol, 4.70% yield, 98.4% purity) as a white solid. LCMS (ES⁺): m/z 419.1 [M+H]⁺.

### Example 148. Synthesis of trans-(S)-1-(2-((4-acetamidocyclohexyl)amino)-5-fluoropyrimidin-4-yl)-N-cyclopropylpiperidine-3-carboxamide (Compound 198)

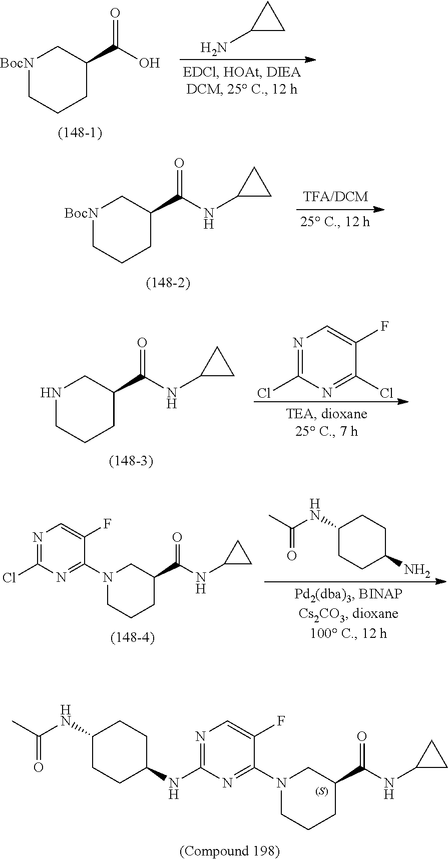

Synthesis of tert-butyl (S)-3-(cyclopropylcarbamoyl)piperidine-1-carboxylate (148-2). To a solution of cyclopropanamine (598 mg, 10.5 mmol, 725 μL, 1.20 eq) in DCM (26 mL) were added EDCI (3.34 g, 17.5 mmol, 2.00 eq), HOAT (1.78 g, 13.1 mmol, 1.83 mL, 1.50 eq), DIEA (3.38 g, 26.2 mmol, 4.56 mL, 3.00 eq) and (S)-1-(tert-butoxycarbonyl)piperidine-3-carboxylic acid (148-1) (2.00 g, 8.72 mmol, 1.00 eq) at 0 °C. The mixture was stirred at 25 °C for 12 h. LC-MS showed ~19.2% of 1-(tert-butoxycarbonyl)piperidine-3-carboxylic acid (148-1) was remaining, and ~10.8% of the desired mass was detected. Water (20 mL) was added to the mixture at 25 °C. The reaction mixture was extracted with DCM (10 mL*5). The organic layers were dried over anhydrous sodium sulfate, filtered and the filtrate was concentrated under reduced to give a residue. The residue was purified by prep-TLC (SiO₂, Ethyl acetate:Petroleum ether=5:1) to give the title compound (148-2) (2.30 g, 8.57 mmol, 98.3% yield) as a yellow oil. LCMS (ES⁺): m/z 269.1 [M+H]⁺

Synthesis of (S)—N-cyclopropylpiperidine-3-carboxamide (148-3). To a solution of tert-butyl (S)-3-(cyclopropylcarbamoyl)piperidine-1-carboxylate (148-2) (1.11 g, 4.14 mmol, 1.00 eq) in DCM (9 mL) was added TFA (4.61 g, 40.4 mmol, 3.00 mL, 9.76 eq) at 25 °C. The mixture was stirred at 25 °C for 12 h. LC-MS showed tert-butyl (S)-3-(cyclopropylcarbamoyl)piperidine-1-carboxylate (148-2) was consumed completely and the desired mass was detected. The reaction mixture was concentrated under reduced pressure to remove solvent to give the title compound (148-3) (1.17 g, crude, TFA) as a yellow oil. LCMS (ES⁺): m/z 169.2 [M+H]⁺.

Synthesis of (S)-1-(2-chloro-5-fluoropyrimidin-4-yl)-N-cyclopropylpiperidine-3-carboxamide (148-4). To a solution of (S)—N-cyclopropylpiperidine-3-carboxamide (148-3) (1.17 g, 4.15 mmol, 1.00 eq, TFA) in dioxane (17 mL) were added 2,4-dichloro-5-fluoropyrimidine (692 mg, 4.15 mmol, 1.00 eq) and TEA (461 mg, 4.56 mmol, 635 μL, 1.10 eq) at 25 °C. The mixture was stirred at 25 °C for 2 h. LC-MS showed ~11% of (S)—N-cyclopropylpiperidine-3-carboxamide (148-3) was remaining, and ~68% of the desired mass was detected. The mixture was stirred at 25 °C for another 2 h. LC-MS showed ~12.8% of (S)—N-cyclopropylpiperidine-3-carboxamide (148-3) was remaining, and ~68.6% of the desired mass was detected. The mixture was stirred at 25 °C for another 3 h. LC-MS showed (S)—N-cyclopropylpiperidine-3-carboxamide (148-3) was consumed completely and the desired mass was detected. Water (15 mL) was added to the mixture at 25 °C. The reaction mixture was extracted with EtOAC (10 mL*5). The organic layers were dried over anhydrous sodium sulfate, filtered and the filtrate was concentrated under reduced to give a residue. The residue was purified by column chromatography (SiO₂, Petroleum ether:Ethyl acetate=1:1 to 1:4) to give the title compound (148-4) (1.10 g, 3.68 mmol, 88.8% yield) as a white solid. LCMS (ES⁺): m/z 299.1 [M+H]⁺.

Synthesis of trans-(S)-1-(2-((4-acetamidocyclohexyl)amino)-5-fluoropyrimidin-4-yl)-N-cyclopropylpiperidine-3-carboxamide (Compound 198). To a solution of (S)-1-(2-chloro-5-fluoropyrimidin-4-yl)-N-cyclopropylpiperidine-3-carboxamide (148-4) (300 mg, 1.00 mmol, 1.00 eq) in dioxane (7 mL) were added trans-N-(4-aminocyclohexyl)acetamide (157 mg, 1.00 mmol, 1.00 eq), Cs₂CO₃ (654 mg, 2.01 mmol, 2.00 eq), BINAP (43.8 mg, 70.3 μmol, 0.0700 eq) and Pd₂(dba)₃ (46.0 mg, 50.2 μmol, 0.0500 eq) at 25 °C under N₂ atmosphere. The mixture was stirred at 100 °C for 12 h under N₂. LC-MS showed ~8.3% of (S)-1-(2-chloro-5-fluoropyrimidin-4-yl)-N-cyclopropylpiperidine-3-carboxamide (148-4) was remaining, and ~15.9% of the desired mass was detected. Water (7 mL) was added to the mixture at 25 °C. The reaction mixture was extracted with EtOAC (5 mL*5). The organic layers were dried over anhydrous sodium sulfate, filtered and the filtrate was concentrated under reduced to give a residue. The residue was purified by prep-HPLC (column: Phenomenex luna C18 100*40 mm*5 um; mobile phase: [H₂O (0.2% FA)-ACN]; gradient: 1%-30% B over 8.0 min) to give the title compound (Compound 198) (10.8 mg, 25.2 μmol, 2.51% yield, 97.7% purity) as a white solid. LCMS (ES⁺): m/z 419.3 [M+H]⁺.

### Example 149. Synthesis of cis-3-((5-fluoro-4-(3-(6-oxo-1,6-dihydropyridin-3-yl)phenyl)pyrimidin-2-yl)amino)cyclohexane-1-carboxamide (Compound 201)

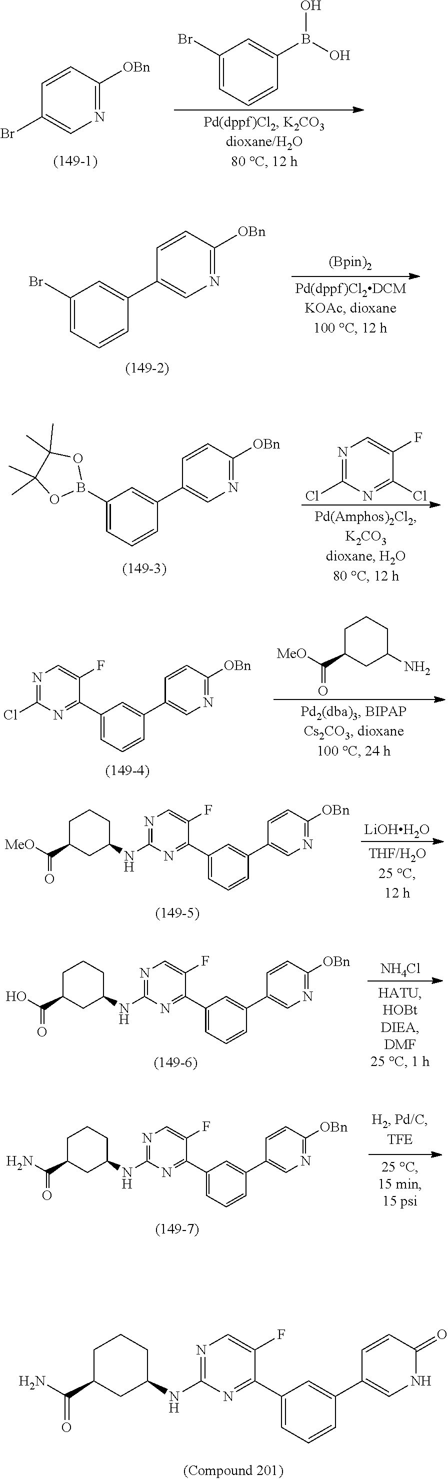

Synthesis of 2-(benzyloxy)-5-(3-bromophenyl)pyridine (149-2). To a stirred solution of (3-bromophenyl)boronic acid (5.30 g, 26.4 mmol, 1.00 eq) and 2-(benzyloxy)-5-bromopyridine (149-1) (6.97 g, 26.39 mmol, 1.00 eq) in dioxane (40 mL) and H₂O (5 mL) were added K₂CO₃ (10.9 g, 79.2 mmol, 3.00 eq) and Pd(dppf)Cl₂ (966 mg, 1.32 mmol, 0.0500 eq) at 25 °C under N₂. The resulting mixture was stirred at 80 °C for 12 h under N₂. LCMS showed 2-(benzyloxy)-5-bromopyridine (149-1) remained little and the desired mass was detected. The mixture was diluted with H₂O (40 mL) and then extracted with EtOAc (40 mL*3). The combined organic layers were dried over Na₂SO₄, filtered and the filtrate was concentrated under reduced pressure to give a residue. The residue was purified by MPLC (SiO₂, PE/EtOAc=1/0 to 5/1) to give the title compound (149-2) (5.20 g, 15.3 mmol, 57.9% yield) as a white solid. LCMS (ES⁺): m/z 339.9 [M+H]⁺.

Synthesis of 2-(benzyloxy)-5-(3-(4,4,5,5-tetramethyl-1,3,2-dioxaborolan-2-yl)phenyl)pyridine (149-3). To a stirred solution of 2-(benzyloxy)-5-(3-bromophenyl)pyridine (149-2) (5.02 g, 14.8 mmol, 1.00 eq) and 4,4,5,5-tetramethyl-2-(4,4,5,5-tetramethyl-1,3,2-dioxaborolan-2-yl)-1,3,2-dioxaborolane (4.87 g, 19.2 mmol, 1.30 eq) in dioxane (100 mL) were added Pd(dppf)Cl₂·CH₂Cl₂ (1.20 g, 1.48 mmol, 0.100 eq) and KOAc (4.34 g, 44.3 mmol, 3.00 eq) at 25 °C under N₂. The resulting mixture was stirred at 100 °C for 12 h under N₂. LCMS showed 2-(benzyloxy)-5-(3-bromophenyl)pyridine (149-2) was consumed completely and the desired mass was detected. The mixture was diluted with H₂O (100 mL) and then extracted with EtOAc (100 mL*3). The combined organic layers were dried over Na₂SO₄, filtered and the filtrate was concentrated under reduced pressure to give a residue. The residue was purified by MPLC (SiO₂, PE/EtOAc=1/0 to 5/1) to give the title compound (149-3) (4.70 g, 12.1 mmol, 82.3% yield) as a white solid. LCMS (ES⁺): m/z 388.1 [M+H]⁺.

Synthesis of 4-(3-(6-(benzyloxy)pyridin-3-yl)phenyl)-2-chloro-5-fluoropyrimidine (149-4). To a stirred solution of 2-(benzyloxy)-5-(3-(4,4,5,5-tetramethyl-1,3,2-dioxaborolan-2-yl)phenyl)pyridine (149-3) (4.22 g, 10.9 mmol, 1.00 eq) and 2,4-dichloro-5-fluoropyrimidine (1.82 g, 10.9 mmol, 1.00 eq) in dioxane (80 mL) and H₂O (8 mL) were added Pd (Amphos)₂Cl₂ (772 mg, 1.09 mmol, 772 μL, 0.100 eq) and K₂CO₃ (4.52 g, 32.7 mmol, 3.00 eq) at 25 °C under N₂. The resulting mixture was stirred at 80 °C for 12 h under N₂. LCMS showed 2-(benzyloxy)-5-(3-(4,4,5,5-tetramethyl-1,3,2-dioxaborolan-2-yl)phenyl)pyridine (149-3) was consumed completely and the desired mass was detected. The mixture was diluted with H₂O (80 mL) and then extracted with EtOAc (80 mL*3). The combined organic layers were dried over Na₂SO₄, filtered and the filtrate was concentrated under reduced pressure. The mixture was purified by MPLC (SiO₂, PE/EtOAc=1/0 to 5/1) to give the title compound (149-4) (1.60 g, 4.08 mmol, 37.5% yield) as yellow solid. LCMS (ES⁺): m/z 392.0 [M+H]⁺.

Synthesis of cis-methyl 3-((4-(3-(6-(benzyloxy)pyridin-3-yl)phenyl)-5-fluoropyrimidin-2-yl)amino)cyclohexane-1-carboxylate (149-5). To a solution of 4-(3-(6-(benzyloxy)pyridin-3-yl)phenyl)-2-chloro-5-fluoropyrimidine (149-4) (300 mg, 766 μmol, 1.00 eq) and cis-methyl 3-aminocyclohexane-1-carboxylate (445 mg, 2.30 mmol, 3.00 eq, HCl) in dioxane (8 mL) were added Pd₂(dba)₃ (70.1 mg, 76.6 μmol, 0.100 eq), Cs₂CO₃ (748 mg, 2.30 mmol, 3.00 eq) and BINAP (47.7 mg, 76.6 μmol, 0.100 eq) at 25 °C under N₂. The mixture was stirred at 100 °C for 12 h under N₂. LCMS showed 4-(3-(6-(benzyloxy)pyridin-3-yl)phenyl)-2-chloro-5-fluoropyrimidine (149-4) remained mostly and the desired mass was detected. To the mixture were added cis-methyl 3-aminocyclohexane-1-carboxylate (445 mg, 2.30 mmol, 3.00 eq, HCl) and Pd₂(dba)₃ (70.1 mg, 76.6 μmol, 0.100 eq) at 25 °C, then the mixture was stirred at 100 °C for another 12 h under N₂. LCMS showed 4-(3-(6-(benzyloxy)pyridin-3-yl)phenyl)-2-chloro-5-fluoropyrimidine (149-4) was consumed mostly and the desired mass was detected. The mixture was diluted with H₂O (10 mL) and then extracted with EtOAc (8 mL*3). The combined organic layers were dried over Na₂SO₄, filtered and the filtrate was concentrated under reduced pressure. The mixture was purified by prep-TLC (SiO₂, EtOAc/MeOH=10/1) to give the title compound (149-5) (120 mg, 234 mol, 30.6% yield) as a yellow solid. LCMS (ES⁺): m/z 513.3 [M+H]⁺.

Synthesis of cis-3-((4-(3-(6-(benzyloxy)pyridin-3-yl)phenyl)-5-fluoropyrimidin-2-yl)amino)cyclohexane-1-carboxylic acid (149-6). To a solution of cis-methyl 3-((4-(3-(6-(benzyloxy)pyridin-3-yl)phenyl)-5-fluoropyrimidin-2-yl)amino)cyclohexane-1-carboxylate (149-5) (120 mg, 234 μmol, 1.00 eq) in THF (2 mL) and H₂O (1 mL) was added LiOH·H₂O (39.3 mg, 936 μmol, 4.00 eq) at 25 °C, the mixture was stirred at 25 °C for 12 h. LCMS showed cis-methyl 3-((4-(3-(6-(benzyloxy)pyridin-3-yl)phenyl)-5-fluoropyrimidin-2-yl)amino)cyclohexane-1-carboxylate (149-5) was consumed completely and the desired mass was detected. To the mixture was concentrated under reduced pressure to remove THF, then the remaining aqueous phase was washed with EtOAc (2 mL*3). Then the aqueous layer was acidified by HCl (1 M) to adjust pH=3-4 and extracted with EtOAc (2 mL*3). The combined organic layers were dried over Na₂SO₄ and then concentrated under reduced pressure to give the title compound (149-6) (110 mg, crude) as a yellow oil. LCMS (ES⁺): m/z 499.2 [M+H]⁺.

Synthesis of cis-3-((4-(3-(6-(benzyloxy)pyridin-3-yl)phenyl)-5-fluoropyrimidin-2-yl)amino)cyclohexane-1-carboxamide (149-7). To a solution of cis-3-((4-(3-(6-(benzyloxy)pyridin-3-yl)phenyl)-5-fluoropyrimidin-2-yl)amino)cyclohexane-1-carboxylic acid (149-6) (110 mg, 221 μmol, 1.00 eq) in DMF (2 mL) were added HOBt (14.9 mg, 110 μmol, 0.500 eq), HATU (100 mg, 264 μmol, 1.20 eq) and DIEA (57.0 mg, 441 μmol, 76.9 μL, 2.00 eq) at 25 °C, then to the reaction was added NH₄Cl (47.2 mg, 883 μmol, 4.00 eq) at 25 °C, the mixture was stirred at 25 °C for 1 h. LCMS showed cis-3-((4-(3-(6-(benzyloxy)pyridin-3-yl)phenyl)-5-fluoropyrimidin-2-yl)amino)cyclohexane-1-carboxylic acid (149-6) was consumed completely and the desired mass was detected. H₂O (3 mL) was added to the solution, the mixture was extracted with EtOAc (3 mL*3), then the combined organic layers were washed with brine (2 mL*3), dried over Na₂SO₄, filtered and the filtrate was concentrated under reduced pressure to give the title compound (149-7) (110 mg, crude) as a white solid. LCMS (ES⁺): m/z 498.2 [M+H]⁺.

Synthesis of cis-3-((5-fluoro-4-(3-(6-oxo-1,6-dihydropyridin-3-yl)phenyl)pyrimidin-2-yl)amino)cyclohexane-1-carboxamide (Compound 201). To a solution of cis-3-((4-(3-(6-(benzyloxy)pyridin-3-yl)phenyl)-5-fluoropyrimidin-2-yl)amino)cyclohexane-1-carboxamide (149-7) (110 mg, 221 μmol, 1.00 eq) in TFE (6 mL) was added 10% Pd/C (110 mg, 50% purity) at 25 °C under N₂. The mixture was stirred at 25 °C under H₂ (15 psi) for 15 min. LCMS showed cis-3-((4-(3-(6-(benzyloxy)pyridin-3-yl)phenyl)-5-fluoropyrimidin-2-yl)amino)cyclohexane-1-carboxamide (149-7) was consumed mostly and the desired mass was detected. The mixture was filtered, the filtrate was concentrate under reduced pressure to give a residue. The residue was purified by prep-HPLC (column: Phenomenex luna C18 100*40 mm*3 um; mobile phase: [H₂O (0.2% FA)-ACN]; gradient: 20%-55% B over 8.0 min) to give the title compound (Compound 201) (8.50 mg, 20.8 μmol, 9.40% yield, 99.6% purity) as yellow solid. LCMS (ES⁺): m/z 408.1 [M+H]⁺.

### Example 150. Synthesis of 5-(3-(2-((1-acetylpiperidin-4-yl)amino)-5-fluoropyrimidin-4-yl)phenyl)pyridin-2(1H)-one (Compound 202)

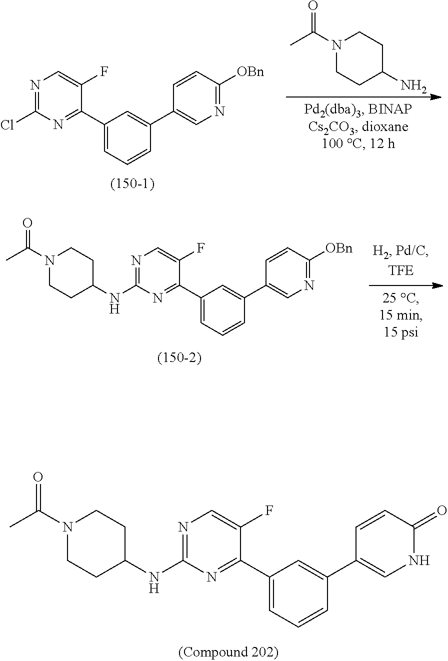

Synthesis of 1-(4-((4-(3-(6-(benzyloxy)pyridin-3-yl)phenyl)-5-fluoropyrimidin-2-yl)amino)piperidin-1-yl)ethan-1-one (150-2). To a solution of 4-(3-(6-(benzyloxy)pyridin-3-yl)phenyl)-2-chloro-5-fluoropyrimidine (150-1) (200 mg, 510 μmol, 1.00 eq) and 1-(4-aminopiperidin-1-yl)ethan-1-one (94.4 mg, 664 μmol, 1.30 eq) in dioxane (5 mL) were added BINAP (19.1 mg, 30.6 μmol, 0.0600 eq), Cs₂CO₃ (333 mg, 1.02 mmol, 2.00 eq) and Pd₂(dba)₃ (18.7 mg, 20.4 μmol, 0.0400 eq) at 25 °C under N₂ atmosphere. The mixture was stirred at 100 °C for 12 h under N₂. LCMS showed 4-(3-(6-(benzyloxy)pyridin-3-yl)phenyl)-2-chloro-5-fluoropyrimidine (150-1) was consumed completely and the desired mass was detected. The mixture was diluted with H₂O (5 mL) and then extracted with EtOAc (5 mL*3). The combined organic layers were dried over Na₂SO₄, filtered and the filtrate was concentrated under reduced pressure. The mixture was purified by prep-TLC (SiO₂, PE/EtOAc=0/1) to give the title compound (150-2) (200 mg, 402 μmol, 78.8% yield) as a white solid. LCMS (ES⁺): m/z 498.2 [M+H]⁺.

Synthesis of 5-(3-(2-((1-acetylpiperidin-4-yl)amino)-5-fluoropyrimidin-4-yl)phenyl)pyridin-2(1H)-one (Compound 202). To a solution of 1-(4-((4-(3-(6-(benzyloxy)pyridin-3-yl)phenyl)-5-fluoropyrimidin-2-yl)amino)piperidin-1-yl)ethan-1-one (150-2) (150 mg, 301 μmol, 1.00 eq) in TFE (3 mL) was added 10% Pd/C (40.0 mg, 50% purity) at 25 °C. The mixture was stirred at 25 °C under H₂ (15 psi) for 15 min. LCMS showed 1-(4-((4-(3-(6-(benzyloxy)pyridin-3-yl)phenyl)-5-fluoropyrimidin-2-yl)amino)piperidin-1-yl)ethan-1-one (150-2) was consumed completely and the desired mass was detected. The mixture was filtered, the filtrate was concentrated under reduced pressure to give a residue. The residue was purified by prep-HPLC (column: Phenomenex Luna C18 100*30 mm*3 um; mobile phase: [H₂O (0.2% FA)-ACN]; gradient: 10%-40% B over 8.0 min) to give the title compound (Compound 202) (33.2 mg, 81.5 μmol, 27.0% yield, 100% purity) as white solid. LCMS (ES⁺): m/z 408.1 [M+H]⁺.

### Example 151. Synthesis of trans-N-(4-((5-fluoro-4-(3-(6-oxo-1,6-dihydropyridin-3-yl)phenyl)pyrimidin-2-yl)amino)cyclohexyl)acetamide (Compound 203)

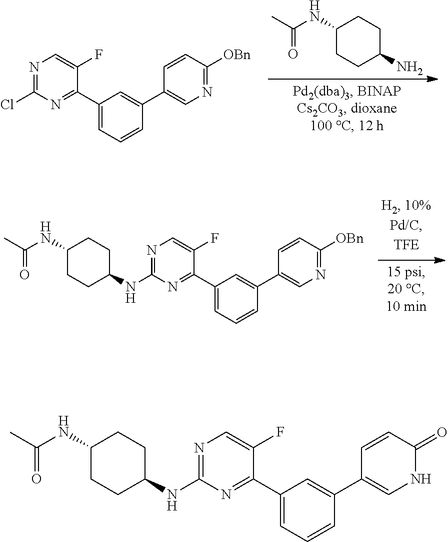 [SMILES: Fc1cnc(Cl)nc1-c1cccc(-c2ccc(OCc3ccccc3)nc2)c1 ; CC(=O)N[C@H]1CC[C@H](N)CC1 ; CC(=O)N[C@H]1CC[C@H](Nc2ncc(F)c(-c3cccc(-c4ccc(OCc5ccccc5)nc4)c3)n2)CC1 ; CC(=O)N[C@H]1CC[C@H](Nc2ncc(F)c(-c3cccc(-c4ccc(=O)[nH]c4)c3)n2)CC1]

Synthesis of trans-N-(4-((4-(3-(6-(benzyloxy)pyridin-3-yl)phenyl)-5-fluoropyrimidin-2-yl)amino)cyclohexyl)acetamide (151-2). To a solution of 4-(3-(6-(benzyloxy)pyridin-3-yl)phenyl)-2-chloro-5-fluoropyrimidine (151-1) (200 mg, 510 μmol, 1.00 eq) and trans-N-(4-aminocyclohexyl)acetamide (159 mg, 1.02 mmol, 2.00 eq) in dioxane (5 mL) were added BINAP (19.1 mg, 30.6 μmol, 0.0600 eq), Cs₂CO₃ (333 mg, 1.02 mmol, 2.00 eq) and Pd₂(dba)₃ (18.7 mg, 20.4 μmol, 0.0400 eq) at 20 °C under N₂. The mixture was stirred at 100 °C for 12 h under N₂. LCMS showed 4-(3-(6-(benzyloxy)pyridin-3-yl)phenyl)-2-chloro-5-fluoropyrimidine (151-1) was consumed and the desired mass was detected. To the mixture was added H₂O (8 mL) and the mixture was extracted with EtOAc (8 mL*3). The combined organic layers were washed with brine (8 mL*3), dried over Na₂SO₄, filtered and the filtrate was concentrated under reduced pressure. The mixture was purified by prep-TLC (SiO₂, EtOAc/MeOH=10/1) to give the title compound (151-2) (200 mg, 391 μmol, 76.6% yield) as a white solid. LCMS (ES⁺): m/z 512.3 [M+H]+.

Synthesis of trans-N-(4-((5-fluoro-4-(3-(6-oxo-1,6-dihydropyridin-3-yl)phenyl)pyrimidin-2-yl)amino)cyclohexyl)acetamide (Compound 203). To a solution of trans-N-(4-((4-(3-(6-(benzyloxy)pyridin-3-yl)phenyl)-5-fluoropyrimidin-2-yl)amino)cyclohexyl)acetamide (151-2) (200 mg, 391 μmol, 1.00 eq) in TFE (3 mL) was added 10% Pd/C (200 mg, 50% purity) at 20 °C. The mixture was stirred at 20 °C for 10 min under H₂ (15 psi) atmosphere. LCMS showed trans-N-(4-((4-(3-(6-(benzyloxy)pyridin-3-yl)phenyl)-5-fluoropyrimidin-2-yl)amino)cyclohexyl)acetamide (151-2) was consumed and the desired mass was detected. The mixture was filtered and the filtrate was concentrated under reduced pressure. The mixture was purified by prep-HPLC (column: Phenomenex luna C18 100*40 mm*5 um; mobile phase: [H₂O (0.2% FA)-ACN]; gradient: 20%-60% B over 8.0 min) to give the title compound (Compound 203) (9.40 mg, 22.3 μmol, 5.71% yield, 100% purity) as a white solid. LCMS (ES⁺): m/z 422.1 [M+H]⁺.

### Example 152. Synthesis of trans-4-((5-fluoro-4-(3-(6-oxo-1,6-dihydropyridin-3-yl)phenyl)pyrimidin-2-yl)amino)cyclohexane-1-carboxamide (Compound 229)

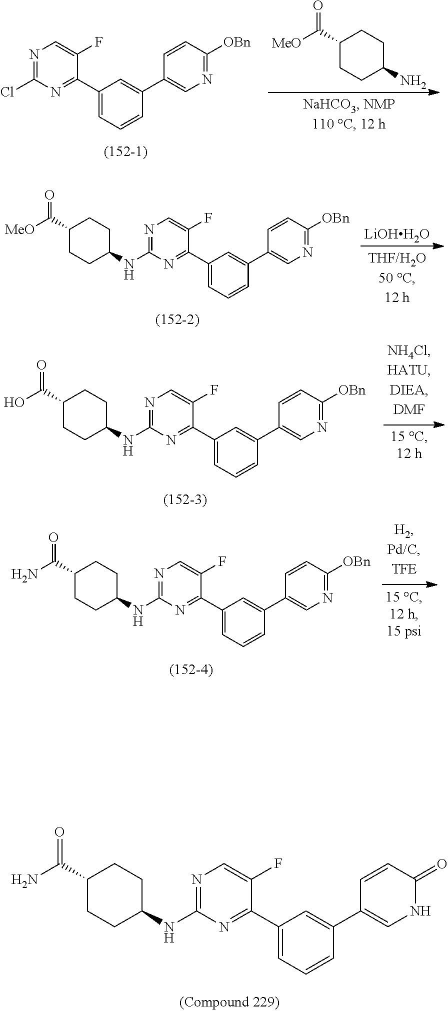

Synthesis of trans-methyl 4-((4-(3-(6-(benzyloxy)pyridin-3-yl)phenyl)-5-fluoropyrimidin-2-yl)amino)cyclohexane-1-carboxylate (152-2). To a solution of 4-(3-(6-(benzyloxy)pyridin-3-yl)phenyl)-2-chloro-5-fluoropyrimidine (152-4) (3.90 g, 9.95 mmol, 1.00 eq) in NMP (50 mL) were added NaHCO₃ (2.51 g, 29.8 mmol, 1.16 mL, 3.00 eq) and trans-methyl 4-aminocyclohexane-1-carboxylate (2.89 g, 14.9 mmol, 1.50 eq, HCl) at 15 °C. The mixture was stirred at 110 °C for 12 h. LC-MS showed that 4-(3-(6-(benzyloxy)pyridin-3-yl)phenyl)-2-chloro-5-fluoropyrimidine (152-1) was consumed completely and the desired mass was detected. The reaction mixture was diluted with water (50 mL) and extracted with EtOAc (50 mL*3). The combined organic layers were washed with brine (25 mL*3), dried over Na₂SO₄, filtered and the filtrate was concentrated under reduced pressure to give a residue. The residue was purified by prep-TLC (SiO₂, Petroleum ether/Ethyl acetate=0/1) to give the title compound (152-2) (3.00 g, 5.85 mmol, 58.8% yield) as a yellow solid. LCMS (ES⁺): m/z 513.2 [M+H]⁺.

Synthesis of trans-4-((4-(3-(6-(benzyloxy)pyridin-3-yl)phenyl)-5-fluoropyrimidin-2-yl)amino)cyclohexane-1-carboxylic acid (152-3). To a solution of trans-methyl 4-((4-(3-(6-(benzyloxy)pyridin-3-yl)phenyl)-5-fluoropyrimidin-2-yl)amino)cyclohexane-1-carboxylate (152-2) (2.00 g, 3.90 mmol, 1.00 eq) in THF (20 mL) was added LiOH·H₂O (327 mg, 7.80 mmol, 2.00 eq) in H₂O (4 mL) at 15 °C. The mixture was stirred at 50 °C for 12 h. TLC (SiO₂, Petroleum ether/Ethyl acetate=0/1) indicated that trans-methyl 4-((4-(3-(6-(benzyloxy)pyridin-3-yl)phenyl)-5-fluoropyrimidin-2-yl)amino)cyclohexane-1-carboxylate (152-2) was consumed completely and new spots formed. The reaction mixture was acidified with 1M HCl to PH<5. The acidified aqueous was extracted with EtOAc (20 mL*3). The combined organic layers were washed with brine (20 mL*3), dried over Na₂SO₄, filtered and the filtrate was concentrated under reduced pressure to give title compound (152-3) (1.00 g, crude) as a yellow solid. LCMS (ES⁺): m/z 499.0 [M+H]⁺.

Synthesis of trans-4-((4-(3-(6-(benzyloxy)pyridin-3-yl)phenyl)-5-fluoropyrimidin-2-yl)amino)cyclohexane-1-carboxamide (152-4). To a solution of trans-4-((4-(3-(6-(benzyloxy)pyridin-3-yl)phenyl)-5-fluoropyrimidin-2-yl)amino)cyclohexane-1-carboxylic acid (152-3) (1.00 g, 2.01 mmol, 1.00 eq) in DMF (10 mL) were added NH₄Cl (161 mg, 3.01 mmol, 1.50 eq), DIEA (777 mg, 6.02 mmol, 1.05 mL, 3.00 eq) and HATU (1.14 g, 3.01 mmol, 1.50 eq) at 15 °C. The mixture was stirred at 15 °C for 2 h. LC-MS showed trans-4-((4-(3-(6-(benzyloxy)pyridin-3-yl)phenyl)-5-fluoropyrimidin-2-yl)amino)cyclohexane-1-carboxylic acid (152-3) was consumed completely and the desired mass was detected. The reaction mixture was diluted with water (10 mL) and extracted with EtOAc (10 mL*3). The combined organic layers were washed with brine (10 mL*3), dried over Na₂SO₄, filtered and the filtrate was concentrated under reduced pressure. The residue was purified by column chromatography (SiO₂, Petroleum ether/Ethyl acetate=3/1 to 1/1) to give title compound (152-4) (800 mg, 1.61 mmol, 80.16% yield) as a yellow solid. LCMS (ES⁺): m/z 498.2 [M+H]⁺.

Synthesis of trans-4-((5-fluoro-4-(3-(6-oxo-1,6-dihydropyridin-3-yl)phenyl)pyrimidin-2-yl)amino)cyclohexane-1-carboxamide (Compound 229). To a mixture of 10% Pd/C (250 mg, 50% purity) in TFE (5 mL) was added trans-4-((4-(3-(6-(benzyloxy)pyridin-3-yl)phenyl)-5-fluoropyrimidin-2-yl)amino)cyclohexane-1-carboxamide (152-7) (500 mg, 1.00 mmol, 1.00 eq) at 15 °C. The mixture was stirred at 15 °C for 12 h under H₂ (15 psi). LC-MS showed trans-4-((4-(3-(6-(benzyloxy)pyridin-3-yl)phenyl)-5-fluoropyrimidin-2-yl)amino)cyclohexane-1-carboxamide (152-7) was consumed completely, and the desired mass was detected. The reaction mixture was filtered and the filtrate was concentrated under reduced pressure to give a residue. The residue was purified by prep-HPLC (column: Phenomenex luna C18 100*40 mm*3 um; mobile phase: [H₂O (0.2% FA)-ACN]; gradient: 20%-50% B over 8.0 min) to give the title compound (Compound 229) (12.9 mg, 31.0 μmol, 3.09% yield, 98.1% purity) as a yellow solid. LCMS (ES⁺): m/z 408.2 [M+H]⁺.

### Example 153. Synthesis of (1R,3S)-3-((5-fluoro-4-(3-(5-methyl-2-oxopyridin-1 (2H)-yl)phenyl)pyrimidin-2-yl)amino)cyclohexane-1-carboxamide (Compound 207)

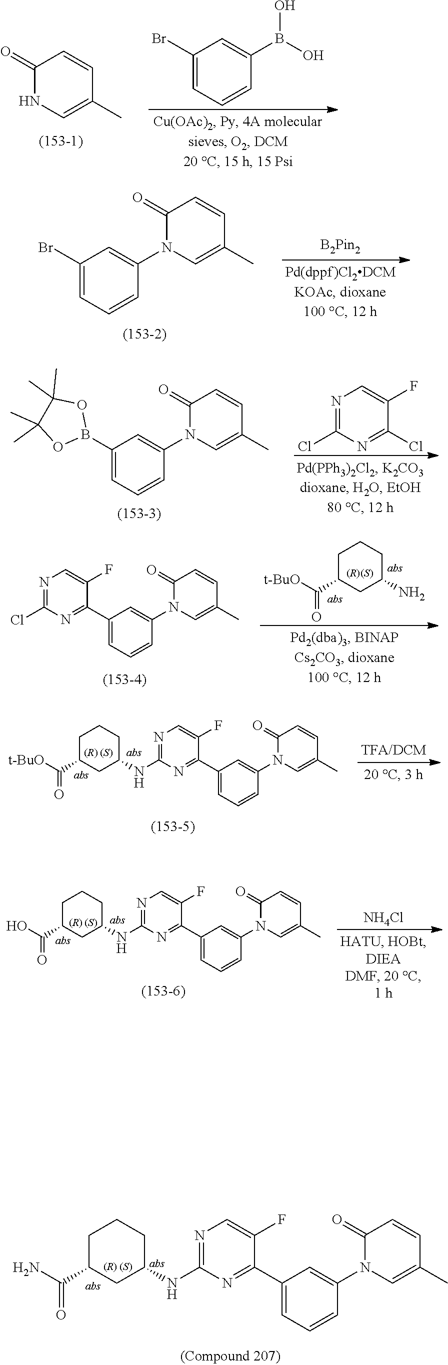

Synthesis of tert-butyl (1R,3S)-3-aminocyclohexane-1-carboxylate (153-2A). To a solution of tert-butyl (1R,3S)-3-(((benzyloxy)carbonyl)amino)cyclohexane-1-carboxylate (153-1A) (3 g, 9.00 mmol, 1 eq) in EtOAc (80 mL) was added 10% Pd/C (6 g, 50% purity) at 20 °C, the mixture was stirred at 20 °C for 12 h under H₂ at 15 Psi. LCMS showed tert-butyl (1R,3S)-3-(((benzyloxy)carbonyl)amino)cyclohexane-1-carboxylate (153-1A) was consumed completely and the desired target MS was detected. The mixture was filtered and the filtrate was concentrated in vacuo to give the title compound (153-2A) (1.7 g, crude) as a brown oil. LCMS (ES⁺): m/z 200.2 [M+H]⁺.

Synthesis of 1-(3-bromophenyl)-5-methylpyridin-2(1H)-one (153-2). To a solution of (3-bromophenyl)boronic acid (18.4 g, 91.6 mmol, 2.00 eq) and 5-methylpyridin-2(1H)-one (153-1) (5.00 g, 45.8 mmol, 1.00 eq) in DCM (200 mL) were added Cu(OAc)₂ (832 mg, 4.58 mmol, 0.100 eq), Py (7.25 g, 91.6 mmol, 7.40 mL, 2.00 eq), 4 A MOLECULAR SIEVE (1.00 g) at 20 °C. The mixture was stirred at 20 °C for 12 h. LCMS showed 5-methylpyridin-2 (1H)-one (153-1) was remained and the desired target MS was detected, then the mixture was stirred at 20 °C for another 3 h under O₂ at 15 Psi. LCMS showed 5-methylpyridin-2 (1H)-one (153-1) was consumed mostly and the desired target MS was detected. H₂O (150 mL) was added to the mixture and the mixture was extracted with DCM (200 mL*3), the combined organic layers were washed with brine (150 mL*3), dried over Na₂SO₄, filtered and the filtrate was concentrated in vacuo to give the crude product to give a residue. The residue was purified by MPLC (SiO₂, Petroleum ether:Ethyl acetate=1/0 to 0/1) to give the title compound (153-2) (10.7 g, 40.5 mmol, 88.42% yield) as a yellow solid. LCMS (ES⁺): m/z 264.0 [M+H]⁺⁻.

Synthesis of 5-methyl-1-(3-(4,4,5,5-tetramethyl-1,3,2-dioxaborolan-2-yl)phenylpyridine-2(1H)-one (153-3). To a solution of 1-(3-bromophenyl)-5-methylpyridin-2 (1H)-one (153-2) (10.7 g, 40.5 mmol, 1.00 eq) and 4,4,5,5-tetramethyl-2-(4,4,5,5-tetramethyl-1,3,2-dioxaborolan-2-yl)-1,3,2-dioxaborolane (13.4 g, 52.7 mmol, 1.30 eq) in dioxane (250 mL) were added KOAc (15.9 g, 162 mmol, 4.00 eq) and Pd(dppf)Cl₂·CH₂Cl₂ (3.31 g, 4.05 mmol, 0.100 eq) at 20 °C under N₂. The mixture was stirred at 100 °C for 12 h under N₂. LCMS showed 1-(3-bromophenyl)-5-methylpyridin-2(1H)-one (153-2) was consumed and the desired target MS was detected. H₂O (150 mL) was added to the mixture and the mixture was extracted with EtOAc (200 mL*3), the combined organic layers were washed with brine (150 mL*3), dried over Na₂SO₄, filtered and the filtrate was concentrated in vacuo to give the crude product. The crude product was purified by MPLC (SiO₂, Petroleum ether:Ethyl acetate=1/0 to 0/1) to give the title compound (153-3) (14.2 g, crude) as a yellow oil. LCMS (ES⁺): m/z 312.2 [M+H]⁺.

Synthesis of 1-(3-(2-chloro-5-fluoropyrimidin-4-yl)phenyl)-5-methylpyridin-2 (1H)-one (153-4). To a solution of 5-methyl-1-(3-(4,4,5,5-tetramethyl-1,3,2-dioxaborolan-2-yl)phenyl)pyridin-2(1H)-one (153-3) (12.0 g, 38.6 mmol, 1.00 eq) and 2,4-dichloro-5-fluoropyrimidine (6.44 g, 38.6 mmol, 1.00 eq) in dioxane (200 mL), H₂O (20 mL) and EtOH (50 mL) were added Pd(PPh₃)₂Cl₂ (271 mg, 387 μmol, 0.01 eq) and K₂CO₃ (16.0 g, 116 mmol, 3.00 eq) at 20 °C under N₂. The mixture was stirred at 80 °C for 12 h under N₂. LCMS showed 5-methyl-1-(3-(4,4,5,5-tetramethyl-1,3,2-dioxaborolan-2-yl)phenyl)pyridin-2(1H)-one (153-3) was consumed and the desired target MS was detected. H₂O (150 mL) was added to the mixture and the mixture was extracted with EtOAc (200 mL*3), the combined organic layers were washed with brine (150 mL*3), dried over Na₂SO₄, filtered and the filtrate was concentrated in vacuo to give the crude product. The crude product was purified by MPLC (SiO₂, Petroleum ether:Ethyl acetate=1/0 to 0/1) to give the title compound (153-4) (10.0 g, 31.7 mmol, 82.1% yield) as a gray solid. LCMS (ES⁺): m/z 316.1 [M+H]⁺.

Synthesis of tert-butyl (1R,3S)-3-((5-fluoro-4-(3-(5-methyl-2-oxopyridin-1 (2H)-yl)phenyl)pyrimidin-2-yl)amino)cyclohexane-1-carboxylate (153-5). To a solution of 1-(3-(2-chloro-5-fluoropyrimidin-4-yl)phenyl)-5-methylpyridin-2(1H)-one (153-4) (1.50 g, 4.75 mmol, 1.00 eq) and tert-butyl (1R,3S)-3-aminocyclohexane-1-carboxylate (1.70 g, 8.55 mmol, 1.80 eq) in dioxane (30 mL) were added BINAP (266 mg, 428 μmol, 0.0900 eq), Pd₂(dba)₃ (218 mg, 238 μmol, 0.0500 eq) and Cs₂CO₃ (4.64 g, 14.3 mmol, 3.00 eq) at 20 °C under N₂. The mixture was stirred at 100 °C for 12 h under N₂ atmosphere. LCMS showed 1-(3-(2-chloro-5-fluoropyrimidin-4-yl)phenyl)-5-methylpyridin-2(1H)-one (153-4) was consumed and the desired target MS was detected. H₂O (30 mL) was added to the mixture and the mixture was extracted with EtOAc (30 mL*3), the combined organic layers were washed with brine (30 mL*3), dried over Na₂SO₄, filtered and the filtrate was concentrated in vacuo to give the crude product. The crude product was purified by MPLC (SiO₂, Petroleum ether:Ethyl acetate=1/0 to 0/1) to give the title compound (153-5) (2.00 g, 4.18 mmol, 88.0% yield) as a gray solid. LCMS (ES⁺): m/z 479.2 [M+H]⁺

Synthesis of (1R,3S)-3-((5-fluoro-4-(3-(5-methyl-2-oxopyridin-1 (2H)-yl)phenyl)pyrimidin-2-yl)amino)cyclohexane-1-carboxylic acid (153-6). A solution of tert-butyl (1R,3S)-3-((5-fluoro-4-(3-(5-methyl-2-oxopyridin-1 (2H)-yl)phenyl)pyrimidin-2-yl)amino)cyclohexane-1-carboxylate (153-5) (600 mg, 1.25 mmol, 1.00 eq) in DCM (10 mL) and TFA (4 mL) was stirred at 20 °C for 2 h. LCMS showed tert-butyl (1R,3S)-3-((5-fluoro-4-(3-(5-methyl-2-oxopyridin-1 (2H)-yl)phenyl)pyrimidin-2-yl)amino)cyclohexane-1-carboxylate (153-5) remained and the desired target MS was detected, then the mixture was stirred at 20 °C for another 1 h. LCMS showed tert-butyl (1R,3S)-3-((5-fluoro-4-(3-(5-methyl-2-oxopyridin-1 (2H)-yl)phenyl)pyrimidin-2-yl)amino)cyclohexane-1-carboxylate (153-5) was consumed mostly and the desired target MS was detected. The mixture was concentrated in vacuo to give the title compound (153-6) (520 mg, crude) as a yellow oil. LCMS (ES⁺): m/z 423.1 [M+H]⁺

Synthesis of (1R,3S)-3-((5-fluoro-4-(3-(5-methyl-2-oxopyridin-1 (2H)-yl)phenyl)pyrimidin-2-yl)amino)cyclohexane-1-carboxamide (Compound 207). To a solution of (1R,3S)-3-((5-fluoro-4-(3-(5-methyl-2-oxopyridin-1 (2H)-yl)phenyl)pyrimidin-2-yl)amino)cyclohexane-1-carboxylic acid (153-6) (300 mg, 710 μmol, 1.00 eq) in DMF (2.5 mL) were added HATU (324 mg, 852 μmol, 1.20 eq), HOBt (48.0 mg, 355 μmol, 0.500 eq), DIEA (91.8 mg, 710 μmol, 124 μL, 1.00 eq) at 20 °C, then NH₄Cl (152 mg, 2.84 mmol, 4.00 eq) was added to the mixture at 20 °C, the mixture was stirred at 20 °C for 1 h. LCMS showed (1R,3S)-3-((5-fluoro-4-(3-(5-methyl-2-oxopyridin-1 (2H)-yl)phenyl)pyrimidin-2-yl)amino)cyclohexane-1-carboxylic acid (153-6) was consumed and the desired target MS was detected. The mixture was concentrated in vacuo to give the crude product. The crude product was purified by prep-HPLC (column: Phenomenex Gemini-NX 80*40 mm*3 um; mobile phase: [H₂O (10 mM NH₄HCO₃)-ACN]; gradient: 25%-55% B over 8.0 min) to give the title compound (Compound 207) (116 mg, 264 μmol, 37.2% yield, 96.5% purity) as a white solid. LCMS (ES⁺): m/z 422.1 [M+H]⁺.

### Example 154. Synthesis of trans-N-(4-((5-fluoro-4-(3-(5-methyl-2-oxopyridin-1 (2H)-yl)phenyl)pyrimidin-2-yl)amino)cyclohexyl)acetamide (Compound 235)

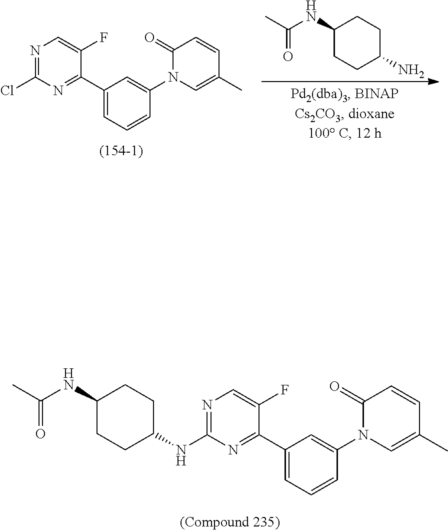

Synthesis of trans-N-(4-((5-fluoro-4-(3-(5-methyl-2-oxopyridin-1 (2H)-yl)phenyl)pyrimidin-2-yl)amino)cyclohexyl)acetamide (Compound 235). To a solution of 1-(3-(2-chloro-5-fluoropyrimidin-4-yl)phenyl)-5-methylpyridin-2(1H)-one (154-1) (500 mg, 1.43 mmol, 1.00 eq) and trans-N-(4-aminocyclohexyl)acetamide (446 mg, 2.85 mmol, 2.00 eq) in dioxane (20 mL) were added BINAP (80.0 mg, 128 μmol, 0.0900 eq), Pd₂(dba)₃ (65.3 mg, 71.3 μmol, 0.0500 eq), Cs₂CO₃ (1.39 g, 4.28 mmol, 3.00 eq) at 20 °C under N₂, the mixture was stirred at 100 °C for 12 h under N₂ atmosphere. LCMS showed 1-(3-(2-chloro-5-fluoropyrimidin-4-yl)phenyl)-5-methylpyridin-2(1H)-one (154-1) was consumed and the desired target MS was detected. H₂O (30 mL) was added to the mixture and the mixture was extracted with EtOAc (30 mL*3), the combined organic layers were washed with brine (30 mL*3), dried over Na₂SO₄, filtered and the filtrate was concentrated in vacuo to give the crude product. The crude product was purified by prep-HPLC (column: Phenomenex luna C18 100*40 mm*3 um; mobile phase: [H₂O (0.2% FA)-ACN]; gradient: 25%-60% B over 8.0 min) to give the title compound (Compound 235) (266.8 mg, 602 μmol, 42.2% yield, 98.2% purity) as a yellow solid. LCMS (ES⁺): m/z 436.2 [M+H]⁺

### Example 155. Synthesis of trans-4-((5-fluoro-4-(3-(5-methyl-2-oxopyridin-1 (2H)-yl)phenyl)pyrimidin-2-yl)amino)cyclohexane-1-carboxamide (Compound 247)

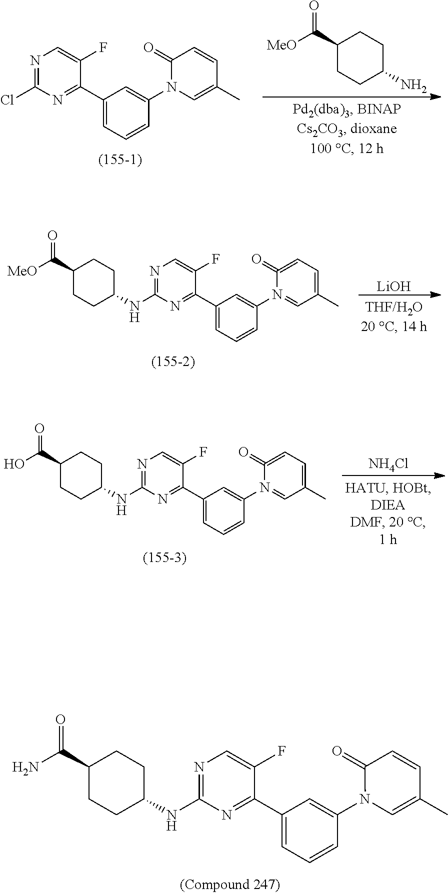

Synthesis of trans-methyl-4-((5-fluoro-4-(3-(5-methyl-2-oxopyridin-1 (2H)-yl)phenyl)pyrimidin-2-yl)amino)cyclohexane-1-carboxylate (155-2). To a solution of 1-(3-(2-chloro-5-fluoropyrimidin-4-yl)phenyl)-5-methylpyridin-2(1H)-one (155-1) (1.00 g, 3.17 mmol, 1.00 eq) and trans-methyl-4-aminocyclohexane-1-carboxylate (896 mg, 5.70 mmol, 1.80 eq) in dioxane (15 mL) were added BINAP (178 mg, 285 μmol, 0.0900 eq), Pd₂ (dba)₃ (145 mg, 158 μmol, 0.0500 eq) and Cs₂CO₃ (3.10 g, 9.50 mmol, 3.00 eq) at 20 °C under N₂. The mixture was stirred at 100 °C for 12 h under N₂ atmosphere. LCMS showed 1-(3-(2-chloro-5-fluoropyrimidin-4-yl)phenyl)-5-methylpyridin-2(1H)-one (155-1) was consumed and the desired target MS was detected. H₂O (15 mL) was added to the mixture and the mixture was extracted with EtOAc (20 mL*3), the combined organic layers were washed with brine (15 mL*3), dried over Na₂SO₄, filtered and the filtrate was concentrated in vacuo to give the crude product. The crude product was purified by MPLC (SiO₂, Petroleum ether:Ethyl acetate=1/0 to 0/1) to give the title compound (155-2) (1.20 g, 2.75 mmol, 86.8% yield) as a yellow solid. LCMS (ES⁺): m/z 437.1 [M+H]⁺.

Synthesis of trans-(4-((5-fluoro-4-(3-(5-methyl-2-oxopyridin-1 (2H)-yl)phenyl)pyrimidin-2-yl)amino)cyclohexane-1-carboxylic acid (155-3). To a solution of trans-methyl-4-((5-fluoro-4-(3-(5-methyl-2-oxopyridin-1 (2H)-yl)phenyl)pyrimidin-2-yl)amino)cyclohexane-1-carboxylate (155-2) (300 mg, 687 μmol, 1.00 eq) in THF (3 mL) and H₂O (1 mL) was added LiOH·H₂O (72.1 mg, 1.72 mmol, 2.50 eq) at 20 °C, then the mixture was stirred at 20 °C for 2 h. LCMS showed methyl (1r,4r)-4-((5-fluoro-4-(3-(5-methyl-2-oxopyridin-1 (2H)-yl)phenyl)pyrimidin-2-yl)amino)cyclohexane-1-carboxylate (155-2) remained and the desired target MS was detected, then the mixture was stirred at 20 °C for another 12 h. LCMS showed trans-methyl-4-((5-fluoro-4-(3-(5-methyl-2-oxopyridin-1 (2H)-yl)phenyl)pyrimidin-2-yl)amino)cyclohexane-1-carboxylate (155-2) was consumed and the desired target MS was detected. The reaction mixture was concentrated in vacuo to remove THF. HCl (1 M) was added to the remaining aqueous phase to adjust pH to 2. Then it was extracted with EtOAc (3 mL*3). The combined organic layers were dried over Na₂SO₄, filtered and the filtrate was concentrated in vacuo to give the title compound (155-3) (220 mg, crude) as a yellow solid. LCMS (ES⁺): m/z 423.2 [M+H]⁺.

Synthesis of trans-4-((5-fluoro-4-(3-(5-methyl-2-oxopyridin-1 (2H)-yl)phenyl)pyrimidin-2-yl)amino)cyclohexane-1-carboxamide (Compound 247). To a solution of trans-4-((5-fluoro-4-(3-(5-methyl-2-oxopyridin-1 (2H)-yl)phenyl)pyrimidin-2-yl)amino)cyclohexane-1-carboxylic acid (155-3) (220 mg, 521 μmol, 1.00 eq) in DMF (2 mL) were added HATU (297 mg, 781 μmol, 1.50 eq), HOBt (35.2 mg, 260 μmol, 0.500 eq) and DIEA (67.3 mg, 521 μmol, 90.7 μL, 1.00 eq) at 20 °C, then NH₄Cl (111 mg, 2.08 mmol, 4.00 eq) was added to the above mixture, the mixture was stirred at 20 °C for 1 h. LCMS showed trans-4-((5-fluoro-4-(3-(5-methyl-2-oxopyridin-1 (2H)-yl)phenyl)pyrimidin-2-yl)amino)cyclohexane-1-carboxylic acid (155-3) was consumed and the desired target MS was detected. The mixture was concentrated in vacuo to give a residue. The residue was purified by prep-HPLC (column: Phenomenex Gemini-NX 80*40 mm*3 um; mobile phase: [H₂O (10 mM NH₄HCO₃)-ACN]; gradient: 10%-40% B over 8.0 min) to give the title compound (Compound 247) (110 mg, 258 μmol, 49.6% yield, 99.4% purity) as a white solid. LCMS (ES⁺): m/z 422.1 [M+H]⁺.

### Example 156. Synthesis of (1R,3S)-3-((5-fluoro-4-(3-(2-oxo-5-(trifluoromethyl)pyridin-1 (2H)-yl)phenyl)pyrimidin-2-yl)amino)cyclohexane-1-carboxamide (Compound 208)

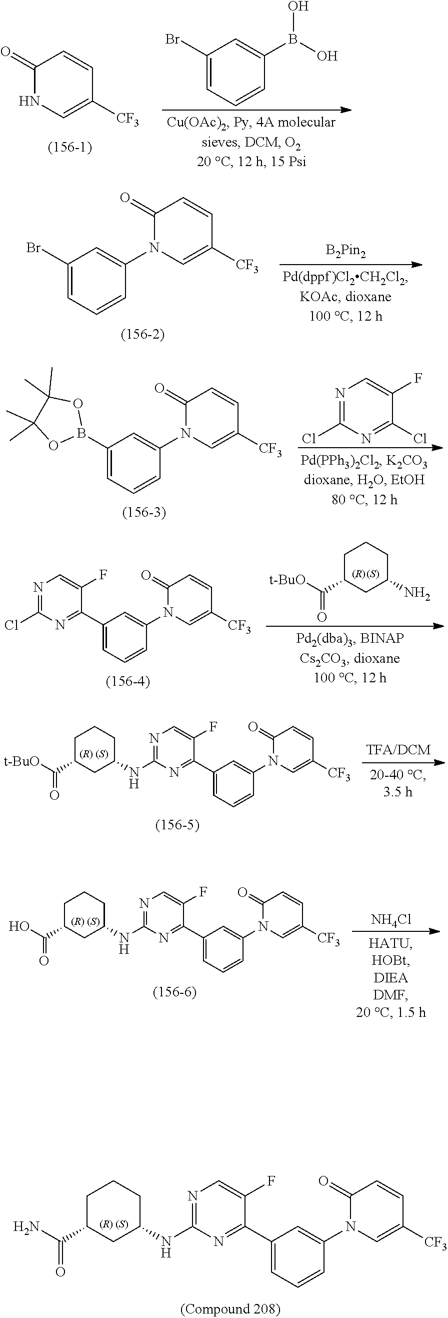

Synthesis of 1-(3-bromophenyl)-5-(trifluoromethyl)pyridin-2(1H)-one (156-2). To a solution of 5-(trifluoromethyl)pyridin-2(1H)-one (156-1) (12.3 g, 61.3 mmol, 2.00 eq) and (3-bromophenyl)boronic acid (5.00 g, 30.7 mmol, 1.00 eq) in DCM (200 mL) were added Py (4.85 g, 61.3 mmol, 4.95 mL, 2.00 eq), 4 A MOLECULAR SIEVE (1.00 g) and Cu(OAc)₂ (557 mg, 3.07 mmol, 0.100 eq) at 20 °C. The mixture was stirred at 20 °C for 12 h under O₂ (15 psi). LCMS showed 5-(trifluoromethyl)pyridin-2(1H)-one (156-1) was consumed and the desired mass was detected. To the mixture was added H₂O (50 mL) and the mixture was extracted with EtOAc (50 mL*3). The combined organic layers were washed with brine (50 mL*3), dried over Na₂SO₄, filtered and the filtrate was concentrated under reduced pressure. The mixture was purified by MPLC (SiO₂, PE/EtOAc=1/0 to 1/1) to give the title compound (156-2) (6.50 g, 20.4 mmol, 66.7% yield) as a yellow oil. LCMS (ES+): m/z 317.9 [M+H]⁺.

Synthesis of 1-(3-(4,4,5,5-tetramethyl-1,3,2-dioxaborolan-2-yl)phenyl)-5-(trifluoromethyl)pyridin-2(1H)-one (156-3). To a solution of 1-(3-bromophenyl)-5-(trifluoromethyl)pyridin-2(1H)-one (156-2) (6.50 g, 20.4 mmol, 1.00 eq) and 4,4,5,5-tetramethyl-2-(4,4,5,5-tetramethyl-1,3,2-dioxaborolan-2-yl)-1,3,2-dioxaborolane (6.75 g, 26.6 mmol, 1.30 eq) in dioxane (50 mL) were added KOAc (6.02 g, 61.3 mmol, 3.00 eq) and Pd(dppf)Cl₂·CH₂Cl₂ (1.67 g, 2.04 mmol, 0.100 eq) at 20 °C under N₂. The mixture was stirred at 100 °C for 12 h under N₂. LCMS showed 1-(3-bromophenyl)-5-(trifluoromethyl)pyridin-2(1H)-one (156-2) was consumed and the desired mass was detected. To the mixture was added H₂O (50 mL) and the mixture was extracted with EtOAc (50 mL*3). The combined organic layers were washed with brine (50 mL*3), dried over Na₂SO₄, filtered and the filtrate was concentrated under reduced pressure to give a residue. The residue was purified by MPLC (SiO₂, PE/EtOAc=1/0 to 1/1) give the title compound (156-3) (7.20 g, 19.7 mmol, 96.5% yield) as a yellow solid. LCMS (ES⁺): m/z 366.2 [M+H]⁺.

Synthesis of 1-(3-(2-chloro-5-fluoropyrimidin-4-yl)phenyl)-5-(trifluoromethyl)pyridin-2(1H)-one (156-4). To a solution of 1-(3-(4,4,5,5-tetramethyl-1,3,2-dioxaborolan-2-yl)phenyl)-5-(trifluoromethyl)pyridin-2(1H)-one (156-3) (3.60 g, 9.86 mmol, 1.00 eq) and 2,4-dichloro-5-fluoropyrimidine (1.65 g, 9.86 mmol, 1.00 eq) in dioxane (100 mL), H₂O (10 mL) and EtOH (25 mL) were added K₂CO₃ (4.09 g, 29.6 mmol, 3.00 eq) and Pd(PPh₃)₂Cl₂ (69.2 mg, 98.6 μmol, 0.0100 eq) at 20 °C under N₂. The mixture was stirred at 80 °C for 12 h under N₂. LCMS showed 1-(3-(4,4,5,5-tetramethyl-1,3,2-dioxaborolan-2-yl)phenyl)-5-(trifluoromethyl)pyridin-2(1H)-one (156-3) was consumed and the desired mass was detected. To the mixture was added H₂O (20 mL) and the mixture was extracted with EtOAc (20 mL*3). The combined organic layers were washed with brine (50 mL*3), dried over Na₂SO₄, filtered and the filtrate was concentrated under reduced pressure to give a residue. The residue was purified by MPLC (SiO₂, PE/EtOAc=1/0 to 1/1) to give the title compound (156-4) (6.70 g, 18.1 mmol, 91.9% yield) as a yellow solid. LCMS (ES⁺): m/z 369.9 [M+H]⁺.

Synthesis of tert-butyl (1R,3S)-3-((5-fluoro-4-(3-(2-oxo-5-(trifluoromethyl)pyridin-1 (2H)-yl)phenyl)pyrimidin-2-yl)amino)cyclohexane-1-carboxylate (156-5). To a solution of 1-(3-(2-chloro-5-fluoropyrimidin-4-yl)phenyl)-5-(trifluoromethyl)pyridin-2(1H)-one (156-4) (1.50, 4.06 mmol, 1.00 eq) and tert-butyl (1R,3S)-3-aminocyclohexane-1-carboxylate (1.46 g, 7.30 mmol, 1.80 eq) in dioxane (20 mL) were added BINAP (227 mg, 365 μmol, 0.0900 eq), Cs₂CO₃ (3.97 g, 12.2 mmol, 3.00 eq) and Pd₂(dba)₃ (186 mg, 203 μmol, 0.0500 eq) at 20 °C under N₂. The mixture was stirred at 100 °C for 12 h under N₂ atmosphere. LCMS showed 1-(3-(2-chloro-5-fluoropyrimidin-4-yl)phenyl)-5-(trifluoromethyl)pyridin-2(1H)-one (156-4) was consumed and the desired mass was detected. To the mixture was added H₂O (20 mL) and the mixture was extracted with EtOAc (20 mL*3). The combined organic layers were washed with brine (20 mL*3), dried over Na₂SO₄, filtered and the filtrate was concentrated under reduced pressure. The mixture was purified by MPLC (SiO₂, PE/EtOAc=1/0 to 1/1) to give the title compound (156-5) (1.90 g, 3.57 mmol, 87.9% yield) as a yellow solid. LCMS (ES⁺): m/z 533.1 [M+H]⁺

Synthesis of (1R,3S)-3-((5-fluoro-4-(3-(2-oxo-5-(trifluoromethyl)pyridin-1 (2H)-yl)phenyl)pyrimidin-2-yl)amino)cyclohexane-1-carboxylic acid (156-6). A solution of tert-butyl (1R,3S)-3-((5-fluoro-4-(3-(2-oxo-5-(trifluoromethyl)pyridin-1 (2H)-yl)phenyl)pyrimidin-2-yl)amino)cyclohexane-1-carboxylate (156-5) (350 mg, 657 μmol, 1.00 eq) in DCM (2.5 mL) and TFA (0.5 mL) was stirred at 20 °C for 2 h. LCMS showed tert-butyl (1R,3S)-3-((5-fluoro-4-(3-(2-oxo-5-(trifluoromethyl)pyridin-1 (2H)-yl)phenyl)pyrimidin-2-yl)amino)cyclohexane-1-carboxylate (156-5) remained and the desired mass was detected. Then the mixture was stirred at 40 °C for another 1.5 h. LCMS showed tert-butyl (1R,3S)-3-((5-fluoro-4-(3-(2-oxo-5-(trifluoromethyl)pyridin-1 (2H)-yl)phenyl)pyrimidin-2-yl)amino)cyclohexane-1-carboxylate (156-5) was consumed completely and the desired mass was detected. The mixture was concentrated under reduced pressure to give the title compound (156-6) (350 mg, crude) as a yellow oil. LCMS (ES⁺): m/z 477.1 [M+H]⁺.

Synthesis of (1R,3S)-3-((5-fluoro-4-(3-(2-oxo-5-(trifluoromethyl)pyridin-1 (2H)-yl)phenyl)pyrimidin-2-yl)amino)cyclohexane-1-carboxamide (Compound 208). To a solution of (1R,3S)-3-((5-fluoro-4-(3-(2-oxo-5-(trifluoromethyl)pyridin-1 (2H)-yl)phenyl)pyrimidin-2-yl)amino)cyclohexane-1-carboxylic acid (156-6) (350 mg, 735 μmol, 1.00 eq) in DMF (3 mL) were added DIEA (190 mg, 1.47 mmol, 256 μL, 2.00 eq), HOBt (49.6 mg, 367 μmol, 0.500 eq), HATU (335 mg, 882 umol, 1.20 eq) and NH₄Cl (157 mg, 2.94 mmol, 4.00 eq) at 20 °C, the mixture was stirred at 20 °C for 1.5 h. LCMS showed (1R,3S)-3-((5-fluoro-4-(3-(2-oxo-5-(trifluoromethyl)pyridin-1 (2H)-yl)phenyl)pyrimidin-2-yl)amino)cyclohexane-1-carboxylic acid (156-6) was consumed and the desired mass was detected. To the mixture was added H₂O (10 mL) and the mixture was extracted with EtOAc (10 mL*3). The combined organic layers were washed with brine (10 mL*3), dried over Na₂SO₄, filtered and the filtrate was concentrated under reduced pressure. The mixture was purified by prep-HPLC (column: Phenomenex luna C18 100*40 mm*3 um; mobile phase: [H₂O (0.2% FA)-ACN]; gradient: 25%-65% B over 8.0 min) to give the title compound (Compound 208) (83.3 mg, 173 μmol, 23.5% yield, 98.5% purity) as yellow solid. LCMS (ES⁺): m/z 476.1 [M+H]⁺

### Example 157. Synthesis of trans-N-(4-((5-fluoro-4-(3-(2-oxo-5-(trifluoromethyl)pyridin-1 (2H)-yl)phenyl)pyrimidin-2-yl)amino)cyclohexyl)acetamide (Compound 234)

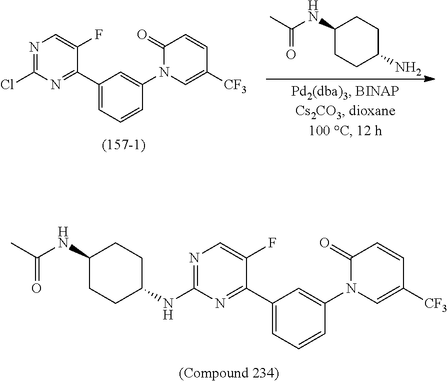 [SMILES: O=c1ccc(C(F)(F)F)cn1-c1cccc(-c2nc(Cl)ncc2F)c1 ; CC(=O)N[C@H]1CC[C@H](N)CC1 ; CC(=O)N[C@H]1CC[C@H](Nc2ncc(F)c(-c3cccc(-n4cc(C(F)(F)F)ccc4=O)c3)n2)CC1]

Synthesis of trans-N-(4-((5-fluoro-4-(3-(2-oxo-5-(trifluoromethyl)pyridin-1 (2H)-yl)phenyl)pyrimidin-2-yl)amino)cyclohexyl)acetamide (Compound 234). To a solution of 1-(3-(2-chloro-5-fluoropyrimidin-4-yl)phenyl)-5-(trifluoromethyl)pyridin-2(1H)-one (157-1) (250 mg, 676 μmol, 1.00 eq) and trans-N-(4-aminocyclohexyl)acetamide (211 mg, 1.35 mmol, 2.00 eq) in dioxane (3 mL) were added BINAP (25.3 mg, 40.6 μmol, 0.0600 eq), Cs₂CO₃ (441 mg, 1.35 mmol, 2.00 eq) and Pd₂(dba)₃ (24.8 mg, 27.1 μmol, 0.0400 eq) at 20 °C under N₂. The mixture was stirred at 100 °C for 12 h under N₂. LCMS showed 1-(3-(2-chloro-5-fluoropyrimidin-4-yl)phenyl)-5-(trifluoromethyl)pyridin-2(1H)-one (157-1) was consumed and the desired mass was detected. To the mixture was added H₂O (10 mL) and the mixture was extracted with EtOAc (10 mL*3). The combined organic layers were washed with brine (10 mL*3), dried over Na₂SO₄, filtered and the filtrate was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 25%-55% B over 8.0 min) to give the title compound (Compound 234) (25.0 mg, 50.6 μmol, 7.49% yield, 99.2% purity) as a white solid. LCMS (ES⁺): m/z 490.2 [M+H]⁺.

### Example 158. Synthesis of trans-4-((5-fluoro-4-(3-(2-oxo-5-(trifluoromethyl)pyridin-1 (2H)-yl)phenyl)pyrimidin-2-yl)amino)cyclohexane-1-carboxamide (Compound 246)

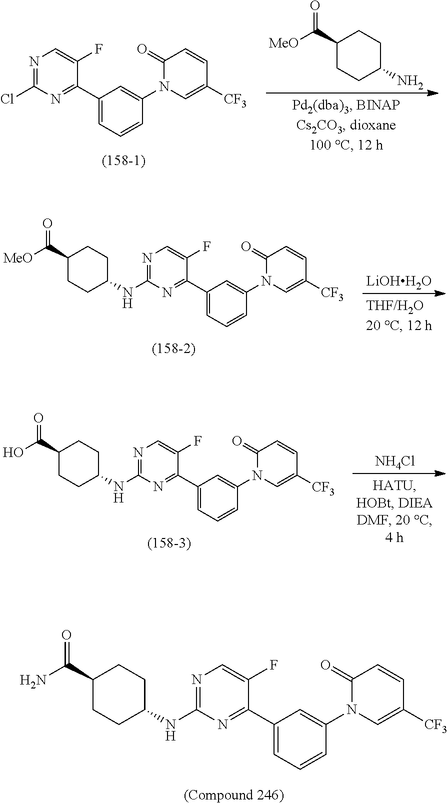

Synthesis of trans-methyl 4-((5-fluoro-4-(3-(2-oxo-5-(trifluoromethyl)pyridin-1 (2H)-yl)phenyl)pyrimidin-2-yl)amino)cyclohexane-1-carboxylate (158-2). To a solution of 1-(3-(2-chloro-5-fluoropyrimidin-4-yl)phenyl)-5-(trifluoromethyl)pyridin-2(1H)-one (158-1) (1.00 g, 2.70 mmol, 1.00 eq) and trans-methyl 4-aminocyclohexane-1-carboxylate (638 mg, 4.06 mmol, 1.50 eq) in dioxane (10 mL) were added BINAP (101 mg, 162 μmol, 0.0600 eq), Cs₂CO₃ (1.76 g, 5.41 mmol, 2.00 eq) and Pd₂(dba)₃ (99.1 mg, 108 μmol, 0.0400 eq) at 20 °C under N₂. The mixture was stirred at 100 °C for 12 h under N₂. LCMS showed 1-(3-(2-chloro-5-fluoropyrimidin-4-yl)phenyl)-5-(trifluoromethyl)pyridin-2(1H)-one (158-1) was consumed and the desired mass was detected. To the mixture was added H₂O (30 mL) and the mixture was extracted with EtOAc (20 mL*3). The combined organic layers were washed with brine (20 mL*3), dried over Na₂SO₄, filtered and the filtrate was concentrated under reduced pressure. The mixture was purified by prep-TLC (SiO₂, PE/EtOAc=1/3) to give the title compound (158-2) (600 mg, 1.22 mmol, 45.2% yield) as a yellow solid. LCMS (ES⁺): m/z 491.3[M+H]⁺.

Synthesis of trans-4-((5-fluoro-4-(3-(2-oxo-5-(trifluoromethyl)pyridin-1 (2H)-yl)phenyl)pyrimidin-2-yl)amino)cyclohexane-1-carboxylic acid (158-3). To a solution of trans-methyl 4-((5-fluoro-4-(3-(2-oxo-5-(trifluoromethyl)pyridin-1 (2H)-yl)phenyl)pyrimidin-2-yl)amino)cyclohexane-1-carboxylate (158-2) (300 mg, 612 μmol, 1.00 eq) in THF (2 mL) and H₂O (1 mL) was added LiOH·H₂O (77.0 mg, 1.84 mmol, 3.00 eq) at 20 °C, the mixture was stirred at 20 °C for 12 h. LCMS showed trans-methyl 4-((5-fluoro-4-(3-(2-oxo-5-(trifluoromethyl)pyridin-1 (2H)-yl)phenyl)pyrimidin-2-yl)amino)cyclohexane-1-carboxylate (158-2) was consumed and the desired mass was detected. To the mixture was added H₂O (5 mL) and the mixture was washed with EtOAc (5 mL*3). Then the aqueous layer was acidified by HCl (1 M) to adjust pH to 4, then it was extracted with EtOAc (5 mL*3). The combined organic layers were dried over Na₂SO₄, filtered and the filtrate was concentrated under reduced pressure to give the title compound (158-3) (180 mg, crude) as yellow oil. LCMS (ES⁺): m/z 477.2 [M+H]⁺.

Synthesis of trans-4-((5-fluoro-4-(3-(2-oxo-5-(trifluoromethyl)pyridin-1 (2H)-yl)phenyl)pyrimidin-2-yl)amino)cyclohexane-1-carboxamide (Compound 246). To a solution of trans-4-((5-fluoro-4-(3-(2-oxo-5-(trifluoromethyl)pyridin-1 (2H)-yl)phenyl)pyrimidin-2-yl)amino)cyclohexane-1-carboxylic acid (158-3) (175 mg, 367 μmol, 1.00 eq) in DMF (2 mL) were added DIEA (95.0 mg, 735 μmol, 128 μL, 2.00 eq), HATU (168 mg, 441 μmol, 1.20 eq), HOBt (24.8 mg, 184 μmol, 0.500 eq) and NH₄Cl (78.6 mg, 1.47 mmol, 4.00 eq) at 20 °C, the mixture was stirred at 20 °C for 4 h. LCMS showed trans-4-((5-fluoro-4-(3-(2-oxo-5-(trifluoromethyl)pyridin-1 (2H)-yl)phenyl)pyrimidin-2-yl)amino)cyclohexane-1-carboxylic acid (158-3) was consumed and the desired mass was detected. The reaction was filtered and the filtrate was concentrated under reduced pressure to give a residue. The residue was purified by prep-HPLC (column: Phenomenex Luna C18 100*30 mm*3 um; mobile phase: [H₂O (0.2% FA)-ACN]; gradient: 30%-60% B over 8.0 min) to give the title compound (Compound 246) (70.4 mg, 147 μmol, 40.1% yield, 99.5% purity) as a white solid. LCMS (ES⁺): m/z 476.1 [M+H]⁺.

### Example 159. Synthesis of cis-3-((5-fluoro-4-(3-(2-oxo-1,3-oxazinan-3-yl)phenyl)pyrimidin-2-yl)amino)cyclohexane-1-carboxamide (Compound 227)

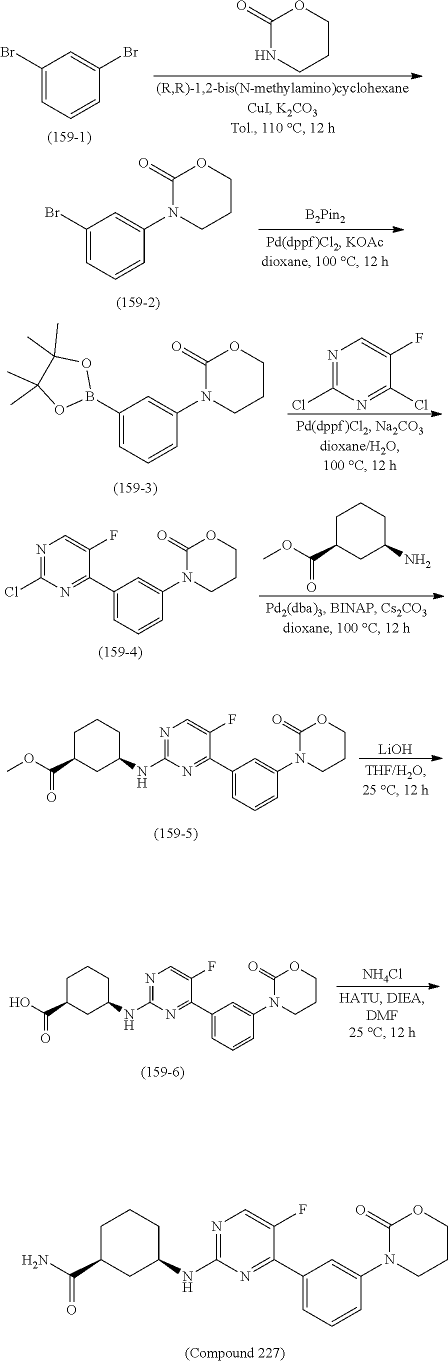

Synthesis of 3-(3-bromophenyl)-1,3-oxazinan-2-one (159-2). A mixture of 1,3-dibromobenzene (159-1) (58.3 g, 247 mmol, 29.8 mL, 2.00 eq), 1,3-oxazinan-2-one (12.5 g, 123 mmol, 1.00 eq), CuI (4.71 g, 24.7 mmol, 0.200 eq), K₂CO₃ (34.1 g, 247 mmol, 2.00 eq) and (1R,2R)—N1,N2-dimethylcyclohexane-1,2-diamine (3.52 g, 24.7 mmol, 0.200 eq) in toluene (80 mL) at 25 °C was degassed and purged with N₂ for 3 times, and then the mixture was stirred at 110 °C for 12 h under N₂ atmosphere. LC-MS showed the desired mass was detected. The reaction mixture was diluted with water 60 mL and extracted with EtOAc (100 mL*3), filtered and concentrated under reduced pressure. The residue was purified by column chromatography (SiO₂, Petroleum ether:Ethyl acetate=1:0 to 0:1) to give the title compound (159-2) (27.6 g, 96.6 mmol, 39.0% yield, 89.6% purity) as a yellow solid. LCMS (ES⁺): m/z 256.2 [M+H]⁺.

Synthesis of 3-[3-(4,4,5,5-tetramethyl-1,3,2-dioxaborolan-2-yl)phenyl]-1,3-oxazinan-2-one (159-3). A mixture of 3-(3-bromophenyl)-1,3-oxazinan-2-one (159-2) (7.00 g, 27.3 mmol, 1.00 eq), 4,4,5,5-tetramethyl-2-(4,4,5,5-tetramethyl-1,3,2-dioxaborolan-2-yl)-1,3,2-dioxaborolane (13.8 g, 54.6 mmol, 2.00 eq), Pd(dppf)Cl₂ (2.00 g, 2.73 mmol, 0.100 eq), KOAc (8.05 g, 82.0 mmol, 3.00 eq) in dioxane (50 mL) at 25 °C was degassed and purged with N₂ for 3 times, and then the mixture was stirred at 100 °C for 12 h under N₂ atmosphere. LC-MS showed the desired mass was detected. The reaction mixture was diluted with water 40 mL and extracted with EtOAc (80 mL*3), filtered and concentrated under reduced pressure. The residue was purified by column chromatography (SiO₂, Petroleum ether:Ethyl acetate=1:0 to 0:1) to give the title compound (159-3) (14.0 g, 28.8 mmol, 52.7% yield, 62.4% purity) as a brown solid. LCMS (ES⁺): m/z 304.1 [M+H]⁺.

Synthesis of 3-[3-(2-chloro-5-fluoro-pyrimidin-4-yl)phenyl]-1,3-oxazinan-2-one (159-4). A mixture of 3-[3-(4,4,5,5-tetramethyl-1,3,2-dioxaborolan-2-yl)phenyl]-1,3-oxazinan-2-one (159-3) (7.00 g, 23.0 mmol, 1.00 eq), 2,4-dichloro-5-fluoro-pyrimidine (3.86 g, 23.0 mmol, 1.00 eq), Pd(dppf)Cl₂ (1.69 g, 2.31 mmol, 0.100 eq), Na₂CO₃ (7.34 g, 69.2 mmol, 3.00 eq) in dioxane (60 mL) and H₂O (20 mL) at 25 °C was degassed and purged with N₂ for 3 times, and then the mixture was stirred at 100 °C for 12 h under N₂ atmosphere. LC-MS showed the desired mass was detected. The reaction mixture was diluted with water 40 mL and extracted with EtOAc (80 mL*3), filtered and concentrated under reduced pressure. The residue was purified by column chromatography (SiO₂, Petroleum ether:Ethyl acetate=1:0 to 0:1) to give the title compound (159-4) (3.55 g, 8.38 mmol, 36.3% yield, 72.6% purity) as a brown solid. LCMS (ES⁺): m/z 308.2 [M+H]⁺.

Synthesis of cis-methyl-3-((5-fluoro-4-(3-(2-oxo-1,3-oxazinan-3-yl)phenyl)pyrimidin-2-yl)amino)cyclohexane-1-carboxylate (159-5). A mixture of 3-[3-(2-chloro-5-fluoro-pyrimidin-4-yl)phenyl]-1,3-oxazinan-2-one (159-4) (300 mg, 974 μmol, 1.00 eq), cis-methyl-3-aminocyclohexane-1-carboxylate (459 mg, 2.92 mmol, 3.00 eq), Pd₂(dba)₃ (89.2 mg, 97.5 μmol, 0.100 eq), BINAP (60.7 mg, 97.5 μmol, 0.100 eq) and Cs₂CO₃ (1.59 g, 4.87 mmol, 5.00 eq) in dioxane (10 mL) at 25 °C was degassed and purged with N₂ for 3 times, and then the mixture was stirred at 100 °C for 12 h under N₂ atmosphere. LC-MS showed the desired mass was detected. The reaction mixture was diluted with water 5 mL and extracted with EtOAc (10 mL*3), filtered and concentrated under reduced pressure. The residue was purified by column chromatography (SiO₂, Dichloromethane:Methanol=1:0 to 10:1) to give the title compound (159-5) (360 mg, 413 μmol, 42.4% yield, 49.2% purity) as a brown solid. LCMS (ES⁺): m/z 429.2 [M+H]⁺.

Synthesis of cis-3-((5-fluoro-4-(3-(2-oxo-1,3-oxazinan-3-yl)phenyl)pyrimidin-2-yl)amino)cyclohexane-1-carboxylic acid (159-6). To a solution of methyl cis-methyl-3-((5-fluoro-4-(3-(2-oxo-1,3-oxazinan-3-yl)phenyl)pyrimidin-2-yl)amino)cyclohexane-1-carboxylate (159-5) (210 mg, 490 μmol, 1.00 eq) in THE (3 mL) and H₂O (1 mL) was added LiOH (35.2 mg, 1.47 mmol, 3.00 eq) at 25 °C. The mixture was stirred at 25 °C for 12 h. LC-MS showed the desired mass was detected. The reaction mixture was concentrated under high vacuum. The reaction mixture was washed with EtOAc (5 mL*3), then the water phase was added HCl (6M) until pH=5. The water phase was concentrated under high vacuum to give the title compound (159-6) (260 mg, crude) as a brown oil. LCMS (ES⁺): m/z 415.2 [M+H]⁺.

Synthesis of cis-3-((5-fluoro-4-(3-(2-oxo-1,3-oxazinan-3-yl)phenyl)pyrimidin-2-yl)amino)cyclohexane-1-carboxamide (Compound 227). To a solution of cis-3-((5-fluoro-4-(3-(2-oxo-1,3-oxazinan-3-yl)phenyl)pyrimidin-2-yl)amino)cyclohexane-1-carboxylic acid (159-6) (210 mg, 506 μmol, 1.00 eq) in DMF (2 mL) was added NH₄Cl (81.3 mg, 1.52 mmol, 3.00 eq), HATU (289 mg, 760 μmol, 1.50 eq) and DIEA (196 mg, 1.52 mmol, 264 μL, 3.00 eq) at 25 °C. The mixture was stirred at 25 °C for 12 h. LC-MS showed the desired mass was detected. The reaction mixture was filtered to give a residue. The residue was purified by prep-HPLC (column: Phenomenex luna C18 100*40 mm*3 um; mobile phase: [H₂O (0.2% FA)-ACN]; gradient: 5%-35% B over 8.0 min) to give the title compound (Compound 227) (16.8 mg, 39.6 μmol, 7.83% yield, 97.7% purity) as a white solid. LCMS (ES+): m/z 414.1 [M+H]+.

### Example 160. Synthesis of 3-[3-[2-[(1-acetyl-4-piperidyl)amino]-5-fluoro-pyrimidin-4-yl]phenyl]-1,3-oxazinan-2-one (Compound 220)

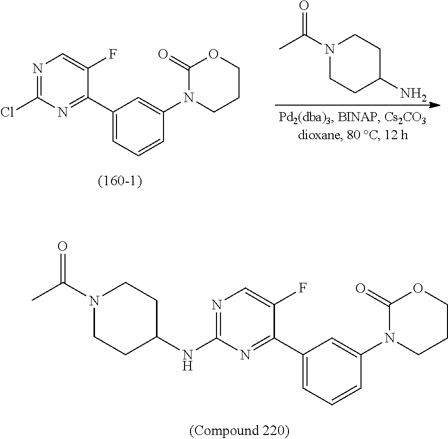

Synthesis of 3-[3-[2-[(1-acetyl-4-piperidyl)amino]-5-fluoro-pyrimidin-4-yl]phenyl]-1,3-oxazinan-2-one (Compound 220). To a solution of 3-[3-(2-chloro-5-fluoro-pyrimidin-4-yl)phenyl]-1,3-oxazinan-2-one (160-1) (150 mg, 487 μmol, 1.00 eq) and 1-(4-amino-1-piperidyl)ethanone (83.1 mg, 584 μmol, 1.20 eq) in dioxane (10 mL) was added Pd₂(dba)₃ (44.6 mg, 48.7 μmol, 0.10 eq), BINAP (30.3 mg, 48.7 μmol, 0.100 eq) and Cs₂CO₃ (476 mg, 1.46 mmol, 3.00 eq) at 25 °C. The mixture was stirred at 80 °C for 12 h under N₂. LC-MS showed (160-1) was consumed completely and the desired mass was detected. The reaction mixture was diluted with water 10 mL and extracted with EtOAc (10 mL*3). The combined organic layers were washed with brine (10 mL*3), dried over Na₂SO₄, filtered and concentrated under reduced pressure. The residue was purified by prep-HPLC (column: Phenomenex Luna C18 75*30 mm*3 um; mobile phase: [H₂O (0.2% FA)-ACN]; gradient: 15%-45% B over 8.0 min) to give the title compound (Compound 220) (71.0 mg, 171 μmol, 35.0% yield, 99.5% purity) as a white solid. LCMS (ES⁺): m/z 414.1 [M+H]⁺.

### Example 161. Synthesis of trans-N-(4-((5-fluoro-4-(3-(2-oxo-1,3-oxazinan-3-yl)phenyl)pyrimidin-2-yl)amino)cyclohexyl)acetamide (Compound 225)

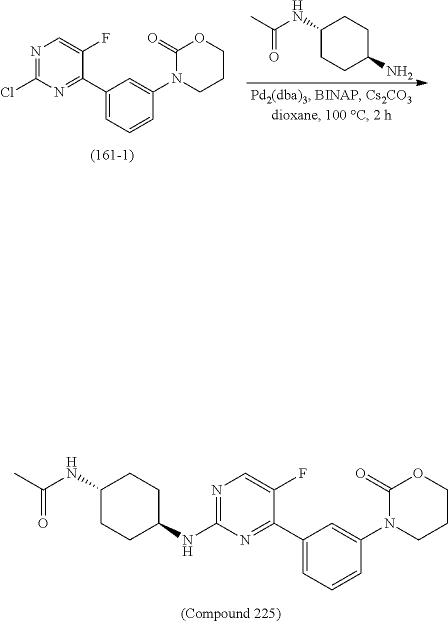 [SMILES: O=C1OCCCN1c1cccc(-c2nc(Cl)ncc2F)c1 ; CC(=O)N[C@H]1CC[C@H](N)CC1 ; CC(=O)N[C@H]1CC[C@H](Nc2ncc(F)c(-c3cccc(N4CCCOC4=O)c3)n2)CC1]

Synthesis of trans-N-(4-((5-fluoro-4-(3-(2-oxo-1,3-oxazinan-3-yl)phenyl)pyrimidin-2-yl)amino)cyclohexyl)acetamide (Compound 225). A mixture of 4-[3-(2-chloro-5-fluoro-pyrimidin-4-yl)phenyl]morpholin-3-one (161-1) (150 mg, 487 μmol, 1.00 eq), trans-N-(4-aminocyclohexyl)acetamide (103 mg, 536 μmol, 1.10 eq, HCl), Cs₂CO₃ (794 mg, 2.44 mmol, 5.00 eq), Pd₂(dba)₃ (44.6 mg, 48.7 μmol, 0.100 eq) and BINAP (30.3 mg, 48.7 μmol, 0.100 eq) in dioxane (3 mL) was degassed and purged with N₂ for 3 times at 25 °C, and then the mixture was stirred at 100 °C for 2 h under N₂ atmosphere. LC-MS showed the desired mass was detected. The mixture was diluted with water (10 mL), and extracted with EtOAc (20 mL*3). The combined organic layers were washed with brine (10 mL), dried over Na₂SO₄, filtered and concentrated under reduced pressure. The residue was purified by prep-HPLC (column: Phenomenex Gemini-NX 100*30 mm*3 um; mobile phase: [H₂O (10 mM NH₄HCO₃)-ACN]; gradient: 5%-45% B over 8.0 min) to give the title compound (Compound 225) (5.00 mg, 11.6 μmol, 2.38% yield, 99.1% purity) as a white solid. LCMS (ES⁺): m/z 428.2 [M+H]⁺.

### Example 162. Synthesis of trans-4-((5-fluoro-4-(3-(2-oxo-1,3-oxazinan-3-yl)phenyl)pyrimidin-2-yl)amino)cyclohexane-1-carboxamide (Compound 236)

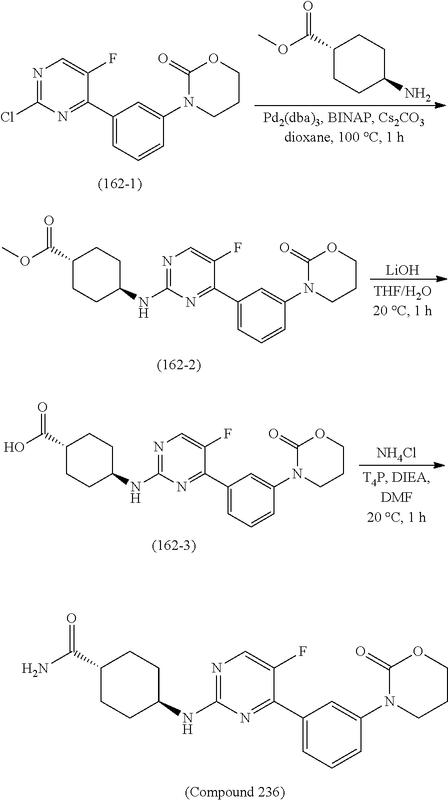

Synthesis of methyl trans-4-((5-fluoro-4-(3-(2-oxo-1,3-oxazinan-3-yl)phenyl)pyrimidin-2-yl)amino)cyclohexane-1-carboxylate (162-2). A mixture of 3-[3-(2-chloro-5-fluoro-pyrimidin-4-yl)phenyl]-1,3-oxazinan-2-one (162-1) (400 mg, 1.30 mmol, 1.00 eq), methyl trans4-aminocyclohexane-1-carboxylate (245 mg, 1.56 mmol, 1.20 eq), BINAP (80.9 mg, 129 μmol, 0.100 eq), Cs₂CO₃ (1.27 g, 3.90 mmol, 3.00 eq) and Pd₂(dba)₃ (119 mg, 129 μmol, 0.100 eq) in dioxane (6 mL) at 20 °C was degassed and purged with N₂ for 3 times, and then the mixture was stirred at 100 °C for 1 h under N₂ atmosphere. LC-MS showed the desired mass was detected. The reaction mixture was diluted with H₂O 10 mL and extracted with EtOAc 10 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (162-2) (250 mg, 337 μmol, 25.9% yield, 57.8% purity) as a yellow solid. LCMS (ES⁺): m/z 429.1 [M+H]⁺.

Synthesis of trans-4-((5-fluoro-4-(3-(2-oxo-1,3-oxazinan-3-yl)phenyl)pyrimidin-2-yl)amino)cyclohexane-1-carboxylic acid (162-3). To a solution of methyl trans-4-((5-fluoro-4-(3-(2-oxo-1,3-oxazinan-3-yl)phenyl)pyrimidin-2-yl)amino)cyclohexane-1-carboxylate (162-2) (100 mg, 233 μmol, 1.00 eq) in THF (2 mL) and H₂O (0.5 mL) was added LiOH (11.1 mg, 466 μmol, 2.00 eq) at 20 °C. The mixture was stirred at 20 °C for 1 h. LC-MS showed the desired mass was detected. The reaction mixture was concentrated under reduced pressure to remove THF. The residue was adjust PH to 6 with AcOH and extracted with EtOAc 5 mL*3. The combined organic layers were dried over Na₂SO₄, filtered and concentrated under reduced pressure to give the title compound (162-3) (100 mg, crude) as a yellow solid. LCMS (ES⁺): m/z 415.2 [M+H]⁺.

Synthesis of trans-4-((5-fluoro-4-(3-(2-oxo-1,3-oxazinan-3-yl)phenyl)pyrimidin-2-yl)amino)cyclohexane-1-carboxamide (Compound 236). To a solution of trans-4-((5-fluoro-4-(3-(2-oxo-1,3-oxazinan-3-yl)phenyl)pyrimidin-2-yl)amino)cyclohexane-1-carboxylic acid (162-3) (100 mg, 241 μmol, 1.00 eq) and NH₄Cl (51.6 mg, 965 μmol, 4.00 eq) in DMF (2 mL) was added T₄P (347 mg, 482 μmol, 50% purity, 2.00 eq) and DIEA (62.3 mg, 482 μmol, 84.0 μL, 2.00 eq) at 20 °C. The mixture was stirred at 20 °C for 1 h. LC-MS showed the desired mass was detected. The reaction mixture was filtered and concentrated under reduced pressure. The residue was purified by prep-HPLC (column: Phenomenex Luna C18 100*30 mm*3 um; mobile phase: [H₂O (0.2% FA)-ACN]; gradient: 1%-50% B over 8.0 min) to give the title compound (Compound 236) (6.2 mg, 14.7 μmol, 6.12% yield, 98.4% purity, 0.15 FA) as a white solid. LCMS (ES⁺): m/z 414.2 [M+H]⁺.

### Example 163. Synthesis of 1-(3-(2-((1-acetylpiperidin-4-yl)amino)-5-fluoropyrimidin-4-yl)phenyl)piperidin-2-one (Compound 221)

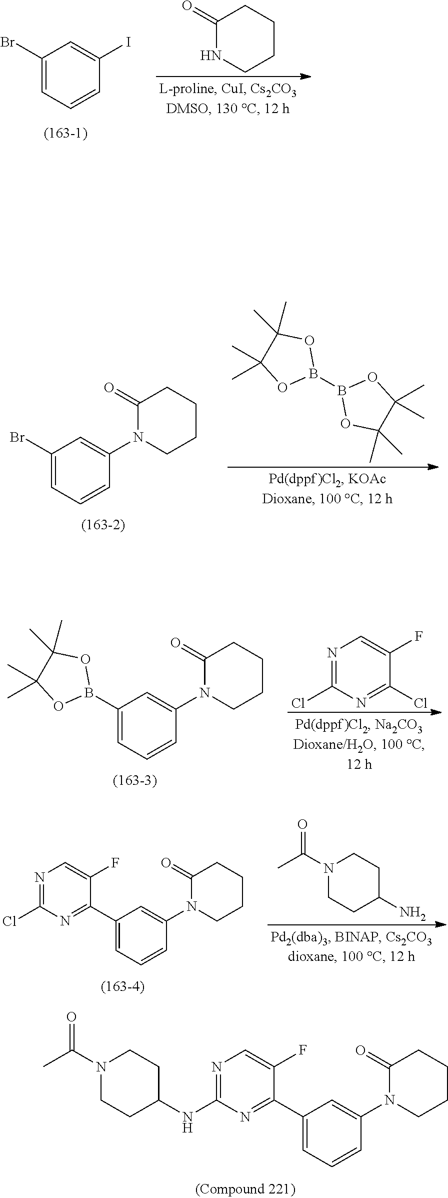

Synthesis of 1-(3-bromophenyl)piperidin-2-one (163-2). A mixture of 1-bromo-3-iodo-benzene (163-1) (7.50 g, 26.5 mmol, 3.38 ml, 1.00 eq), piperidin-2-one (3.15 g, 31.8 mmol, 1.20 eq), L-PROLINE (305 mg, 2.65 mmol, 0.100 eq), Cs₂CO₃ (25.9 g, 79.5 mmol, 3.00 eq) and CuI (504 mg, 2.65 mmol, 0.100 eq) in DMSO (150 mL) was degassed and purged with N₂ for 3 times, and then the mixture was stirred at 130 °C for 12 h under N₂ atmosphere. LC-MS showed the desired mass was detected. The reaction mixture was diluted with H₂O 100 mL and extracted with EtOAc 100 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (163-2) (3.00 g, 10.7 mmol, 20.33% yield, 91.3% purity) as a yellow solid. LCMS (ES⁺): m/z 254.1 [M+H]⁺.

Synthesis of 1-(3-(4,4,5,5-tetramethyl-1,3,2-dioxaborolan-2-yl)phenyl)piperidin-2-one (163-3). A mixture of 1-(3-bromophenyl)piperidin-2-one (163-2) (3.00 g, 11.8 mmol, 1.00 eq), 4,4,5,5-tetramethyl-2-(4,4,5,5-tetramethyl-1,3,2-dioxaborolan-2-yl)-1,3,2-dioxaborolane (3.60 g, 14.1 mmol, 1.20 eq), KOAc (2.32 g, 23.6 mmol, 2.00 eq), Pd(dppf)Cl₂ (863 mg, 1.18 mmol, 0.100 eq) in dioxane (50 mL) at 20 °C was degassed and purged with N₂ for 3 times, and then the mixture was stirred at 100 °C for 12 h under N₂ atmosphere. LC-MS showed the desired mass was detected. The reaction mixture was diluted with H₂O 100 mL and extracted with EtOAc 100 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (163-3) (5.00 g, 9.31 mmol, 78.8% yield, 56.1% purity) as a yellow solid. LCMS (ES⁺): m/z 302.2 [M+H]⁺.

Synthesis of 1-(3-(2-chloro-5-fluoropyrimidin-4-yl)phenyl)piperidin-2-one (163-4). A mixture of 1-[3-(4,4,5,5-tetramethyl-1,3,2-dioxaborolan-2-yl)phenyl]piperidin-2-one (163-3) (5.00 g, 16.6 mmol, 1.00 eq), 2,4-dichloro-5-fluoro-pyrimidine (3.33 g, 19.9 mmol, 1.20 eq), Na₂CO₃ (3.52 g, 33.2 mmol, 2.00 eq), Pd(dppf)Cl₂ (1.21 g, 1.66 mmol, 0.100 eq) in dioxane (30 mL) and H₂O (6 mL) was degassed and purged with N₂ for 3 times, and then the mixture was stirred at 100 °C for 12 h under N₂ atmosphere. LC-MS showed the desired mass was detected. The reaction mixture was diluted with H₂O 100 mL and extracted with EtOAc 100 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (163-4) (5.00 g, 15.3 mmol, 92.3% yield, 93.6% purity) as a yellow solid. LCMS (ES+): m/z 306.1 [M+H]⁺.

Synthesis of 1-(3-(2-((1-acetylpiperidin-4-yl)amino)-5-fluoropyrimidin-4-yl)phenyl)piperidin-2-one (Compound 221). A mixture of 1-[3-(2-chloro-5-fluoro-pyrimidin-4-yl)phenyl]piperidin-2-one (163-4) (300 mg, 981 μmol, 1.00 eq), 1-(4-amino-1-piperidyl)ethanone (167 mg, 1.18 mmol, 1.20 eq), BINAP (61.1 mg, 98.1 μmol, 0.100 eq), Cs₂CO₃ (639 mg, 1.96 mmol, 2.00 eq) and Pd₂(dba)₃ (89.8 mg, 98.1 μmol, 0.100 eq) in dioxane (3 mL) at 20 °C was degassed and purged with N₂ for 3 times, and then the mixture was stirred at 100 °C for 12 h under N₂ atmosphere. LC-MS showed the desired mass was detected. The reaction mixture was diluted with H₂O 10 mL and extracted with EtOAc 10 mL*3. The combined organic layers were dried over Na₂SO₄, filtered and concentrated under reduced pressure. The residue was purified by prep-HPLC (column: Phenomenex Luna C18 75*30 mm*3 um; mobile phase: [H₂O (0.1% TFA)-ACN]; gradient: 15%-45% B over 8.0 min) to give the title compound (Compound 221) (182 mg, 442 μmol, 45.0% yield, 100% purity) as a light yellow solid. LCMS (ES⁺): m/z 412.1 [M+H]⁺.

### Example 164. Synthesis of trans-N-(4-((5-fluoro-4-(3-(2-oxopiperidin-1-yl)phenyl)pyrimidin-2-yl)amino)cyclohexyl)acetamide (Compound 256)

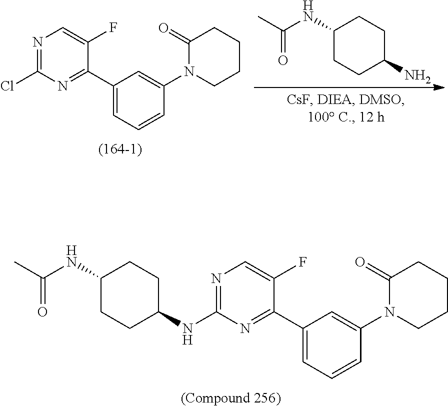

Synthesis of trans-N-(4-((5-fluoro-4-(3-(2-oxopiperidin-1-yl)phenyl)pyrimidin-2-yl)amino)cyclohexyl)acetamide (Compound 256). To a solution of 1-[3-(2-chloro-5-fluoro-pyrimidin-4-yl)phenyl]piperidin-2-one (164-1) (140 mg, 457 μmol, 1.00 eq) in DMSO (5 mL) was added trans-N-(4-aminocyclohexyl)acetamide (107 mg, 686 μmol, 1.50 eq), DIEA (177 mg, 1.37 mmol, 239 μL, 3.00 eq) and CsF (104 mg, 686 μmol, 25.3 μL, 1.50 eq) at 25 °C. The mixture was stirred at 100 °C for 12 h. LC-MS showed the desired mass was detected. The reaction mixture was concentrated under reduced pressure. The residue was purified by prep-HPLC (column: Phenomenex Luna C18 100*30 mm*3 um; mobile phase: [H₂O (0.2% FA)-ACN]; gradient: 15%-45% B over 8.0 min) to give the title compound (Compound 256) (63.2 mg, 147 μmol, 32.2% yield, 99.4% purity) as an off-white solid. LCMS (ES⁺): m/z 426.2 [M+H]⁺.

### Example 165. Synthesis of trans-4-((5-fluoro-4-(3-(2-oxopiperidin-1-yl)phenyl)pyrimidin-2-yl)amino)cyclohexane-1-carboxamide (Compound 264)

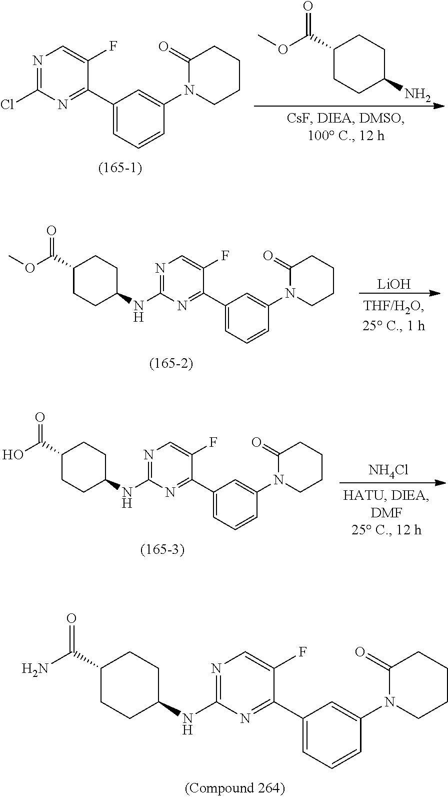

Synthesis of trans-methyl-4-((5-fluoro-4-(3-(2-oxopiperidin-1-yl)phenyl)pyrimidin-2-yl)amino)cyclohexane-1-carboxylate (165-2). To a solution of 1-[3-(2-chloro-5-fluoro-pyrimidin-4-yl)phenyl]piperidin-2-one (165-1) (450 mg, 1.47 mmol, 1.00 eq) in DMSO (5 mL) was added trans-methyl 4-aminocyclohexane-1-carboxylate (347 mg, 2.21 mmol, 1.50 eq), CsF (335 mg, 2.21 mmol, 81.50 μL, 1.50 eq) and DIEA (570 mg, 4.42 mmol, 769.12 μL, 3.00 eq) at 25 °C. The mixture was stirred at 100 °C for 12 h. LC-MS showed desired mass was detected. The reaction mixture was diluted with H₂O 30 mL and extracted with EtOAc (30 mL*3). The combined organic layers were concentrated under reduced pressure. The residue was purified by column chromatography (SiO₂, Petroleum ether:Ethyl acetate=0:1 to 1:1) to give a residue to give the title compound (165-2) (500 mg, 801 μmol, 54.4% yield, 68.3% purity) as a yellow oil. LCMS (ES⁺): m/z 427.2 [M+H]⁺.

Synthesis of trans-4-((5-fluoro-4-(3-(2-oxopiperidin-1-yl)phenyl)pyrimidin-2-yl)amino)cyclohexane-1-carboxylic acid (165-3). To a solution of trans-methyl-4-((5-fluoro-4-(3-(2-oxopiperidin-1-yl)phenyl)pyrimidin-2-yl)amino)cyclohexane-1-carboxylate (165-2) (400 mg, 937 μmol, 1.00 eq) in THF (3 mL) and H₂O (1 mL) was added LiOH (44.9 mg, 1.88 mmol, 2.00 eq) at 25 °C. The mixture was stirred at 25 °C for 1 h. LC-MS showed desired mass was detected. The mixture was adjust pH to 6 with AcOH and extracted with EtOAc 10 mL*3. The combined organic layers were concentrated under reduced pressure to give a residue to give the title compound (165-3) (350 mg, crude) as a yellow solid. LCMS (ES⁺): m/z 413.2 [M+H]⁺.

Synthesis of trans-4-((5-fluoro-4-(3-(2-oxopiperidin-1-yl)phenyl)pyrimidin-2-yl)amino)cyclohexane-1-carboxamide (Compound 264). To a solution of trans-4-((5-fluoro-4-(3-(2-oxopiperidin-1-yl)phenyl)pyrimidin-2-yl)amino)cyclohexane-1-carboxylic acid (165-3) (320 mg, 775 μmol, 1.00 eq) in DMF (5 mL) was added NH₄Cl (83.0 mg, 1.55 mmol, 2.00 eq), DIEA (300 mg, 2.33 mmol, 405.41 μL, 3.00 eq) and HATU (442 mg, 1.16 mmol, 1.50 eq) at 25 °C. The mixture was stirred at 25 °C for 12 h. LC-MS showed desired mass was detected. The reaction mixture was concentrated under reduced pressure. The residue was purified by prep-HPLC (column: Phenomenex luna C18 100*40 mm*3 um; mobile phase: [H₂O (0.2% FA)-ACN]; gradient: 15%-45% B over 8.0 min) to give the title compound (Compound 264) (69.7 mg, 169 μmol, 21.8% yield, 100% purity) as a white solid. LCMS (ES⁺): m/z 412.2 [M+H]⁺.

### Example 166. Synthesis of trans-N-(4-((5-fluoro-4-(3-(3-oxomorpholino)phenyl)pyrimidin-2-yl)amino)cyclohexyl)acetamide (Compound 267)

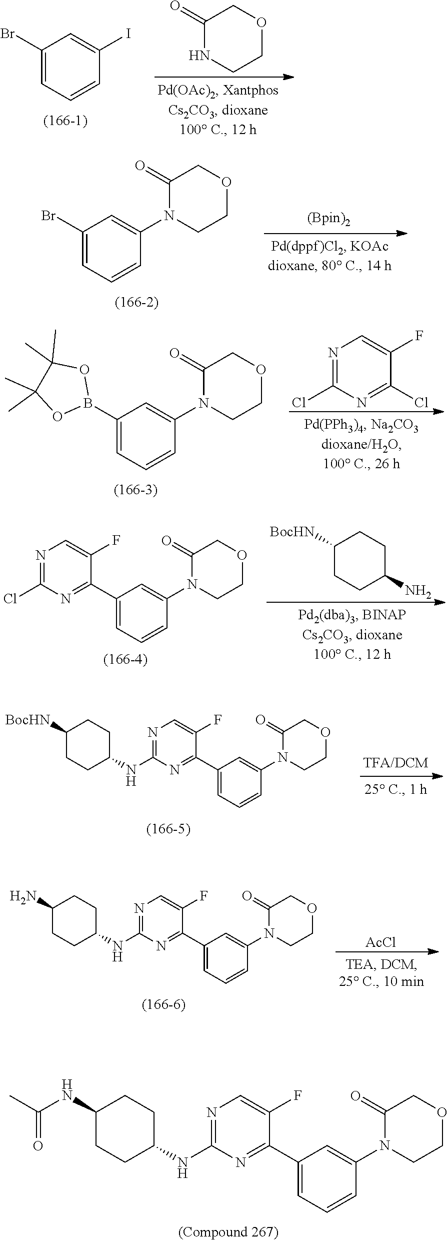

Synthesis of 4-(3-bromophenyl)morpholin-3-one (166-2). To a solution of 1-bromo-3-iodobenzene (166-1) (50.0 g, 177 mmol, 22.5 mL, 1.00 eq) and morpholin-3-one (16.3 g, 161 mmol, 0.900 eq) in dioxane (500 mL) were added Cs₂CO₃ (101 g, 309 mmol, 1.75 eq), Xantphos (13.3 g, 23.0 mmol, 0.130 eq) and Pd(OAc)₂ (3.41 g, 15.2 mmol, 0.0860 eq) under N₂ atmosphere at 25 °C. The mixture was stirred at 100 °C for 12 h under N₂. LCMS showed the desired mass was detected. The reaction mixture was filtered and the filtrate was concentrated under reduced pressure to give a residue. Water (300 mL) was added to the residue at 25 °C. Then the residue was extracted with EtOAc (200 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=2:1 to 1:2) to give the title compound (166-2) (31.0 g, 104 mmol, 58.6% yield, 85.6% purity) as a yellow solid. LCMS (ES⁺): m/z 256.1 [M+H]⁺.

Synthesis of 4-(3-(4,4,5,5-tetramethyl-1,3,2-dioxaborolan-2-yl)phenyl)morpholin-3-one (166-3). To a solution of 4-(3-bromophenyl)morpholin-3-one (166-2) (1.00 g, 3.90 mmol, 1.00 eq) and 4,4,5,5-tetramethyl-2-(4,4,5,5-tetramethyl-1,3,2-dioxaborolan-2-yl)-1,3,2-dioxaborolane (2.97 g, 11.7 mmol, 3.00 eq) in dioxane (30 mL) were added KOAc (1.53 g, 15.6 mmol, 4.00 eq) and Pd(dppf)Cl₂ (285.7 mg, 391 μmol, 0.100 eq) at 25 °C under N₂ atmosphere. The mixture was stirred at 80 °C for 12 h under N₂. LCMS showed 4-(3-bromophenyl)morpholin-3-one (166-2) remained, and the desired mass was detected. Then 4,4,5,5-tetramethyl-2-(4,4,5,5-tetramethyl-1,3,2-dioxaborolan-2-yl)-1,3,2-dioxaborolane (2.97 g, 11.7 mmol, 3.00 eq), KOAc (1.53 g, 15.6 mmol, 4.00 eq) and Pd(dppf)Cl₂ (285.7 mg, 391 μmol, 0.100 eq) were added to the reaction mixture at 25 °C under N₂ atmosphere. The mixture was stirred at 80 °C for another 2 h under N₂. LCMS showed 4-(3-bromophenyl)morpholin-3-one (166-2) remained 10%, and the desired mass was detected. The mixture was not worked up and used to next step directly. The title compound (166-3) (1.00 g, crude) in dioxane (30 mL) was obtained as a black liquid. LCMS (ES⁺): m/z 304.2 [M+H]⁺.

Synthesis of 4-(3-(2-chloro-5-fluoropyrimidin-4-yl)phenyl)morpholin-3-one (166-4). To a solution of 4-(3-(4,4,5,5-tetramethyl-1,3,2-dioxaborolan-2-yl)phenyl)morpholin-3-one (166-3) (1.00 g, 3.30 mmol, 1.00 eq) and 2, 4-dichloro-5-fluoro-pyrimidine (661 mg, 3.96 mmol, 1.20 eq) in dioxane (30.00 mL) and H₂O (6.00 mL) were added Na₂CO₃ (699 mg, 6.60 mmol, 2.00 eq) and Pd(PPh₃)₄ (76.2 mg, 66.0 μmol, 0.0200 eq) at 25 °C under N₂ atmosphere. The mixture was stirred at 90 °C for 12 h under N₂. LCMS showed 4-(3-(4,4,5,5-tetramethyl-1,3,2-dioxaborolan-2-yl)phenyl)morpholin-3-one (166-3) remained, and the desired mass was detected. Then 2, 4-dichloro-5-fluoro-pyrimidine (661 mg, 3.96 mmol, 1.20 eq), Na₂CO₃ (699 mg, 6.60 mmol, 2.00 eq) and Pd(PPh₃)₄ (76.2 mg, 66.0 μmol, 0.0200 eq) were added to the reaction mixture at 25 °C under N₂ atmosphere. The mixture was stirred at 100 °C for another 14 h under N₂. LCMS showed 4-(3-(4,4,5,5-tetramethyl-1,3,2-dioxaborolan-2-yl)phenyl)morpholin-3-one (166-3) was consumed, and the desired mass was detected. The reaction mixture was filtered and the filtrate was concentrated under reduced pressure to give a residue. Water (30 mL) was added to the residue at 25 °C. Then the reaction mixture was extracted with EtOAc (30 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=2:1 to 1:3) to give the title compound (166-4) (900 mg, 2.01 mmol, 60.8% yield, 68.59% purity) as a yellow solid. LCMS (ES⁺): m/z 308.0 [M+H]⁺.

Synthesis of trans-tert-butyl (4-((5-fluoro-4-(3-(3-oxomorpholino)phenyl)pyrimidin-2-yl)amino)cyclohexyl)carbamate (166-5). To a solution of 4-(3-(2-chloro-5-fluoropyrimidin-4-yl)phenyl)morpholin-3-one (166-4) (800 mg, 2.60 mmol, 1.00 eq) and trans-tert-butyl N-(4-aminocyclohexyl)carbamate (1.11 g, 5.20 mmol, 2.00 eq) in dioxane (10 mL) were added BINAP (162 mg, 260 μmol, 0.100 eq), Cs₂CO₃ (2.54 g, 7.80 mmol, 3.00 eq) and Pd₂(dba)₃ (238 mg, 260 μmol, 0.100 eq) at 25 °C under N₂ atmosphere. The mixture was stirred at 100 °C for 12 h under N₂. LCMS showed 4-(3-(2-chloro-5-fluoropyrimidin-4-yl)phenyl)morpholin-3-one (166-4) was consumed completely and the desired mass was detected. The mixture was allowed to room temperature and filtered, the filtrate was concentrated under reduced pressure to give a residue. Water (15 mL) was added to the residue at 25 °C. Then the reaction mixture was extracted with EtOAc (10 mL*3). The organic layers were dried over anhydrous sodium sulfate, filtered and the filtrate was concentrated under reduced pressure to give a residue. The residue was purified by prep-TLC (SiO₂, Petroleum ether:Ethyl acetate=1:2) to give the title compound (166-5) (630 mg, 777 μmol, 29.9% yield, 59.9% purity) as a yellow solid. LCMS (ES⁺): m/z 486.2 [M+H]⁺.

Synthesis of trans-4-(3-(2-((-4-aminocyclohexyl)amino)-5-fluoropyrimidin-4-yl)phenyl)morpholin-3-one (166-6). To a solution of trans-tert-butyl (4-((5-fluoro-4-(3-(3-oxomorpholino)phenyl)pyrimidin-2-yl)amino)cyclohexyl)carbamate (166-5) (300 mg, 618 μmol, 1.00 eq) in DCM (3.00 mL) was added TFA (1.54 g, 13.5 mmol, 1.00 mL, 21.8 eq) at 25 °C. The mixture was stirred at 25 °C for 1 h. LCMS showed trans-tert-butyl (4-((5-fluoro-4-(3-(3-oxomorpholino)phenyl)pyrimidin-2-yl)amino)cyclohexyl)carbamate (166-5) was consumed completely and the desired mass was detected. The reaction mixture was concentrated under reduced pressure to give the title compound (166-6) (300 mg, crude, TFA) as a yellow oil. LCMS (ES⁺): m/z 386.2 [M+H]⁺.

Synthesis of trans-N-(4-((5-fluoro-4-(3-(3-oxomorpholino)phenyl)pyrimidin-2-yl)amino)cyclohexyl)acetamide (Compound 267). To a solution of trans-4-(3-(2-((4-aminocyclohexyl)amino)-5-fluoropyrimidin-4-yl)phenyl)morpholin-3-one (166-6) (300 mg, 601 μmol, 1.00 eq, TFA) in DCM (3.00 mL) was added TEA (182 mg, 1.80 mmol, 251 μL, 3.00 eq) at 25 °C, then acetyl chloride (37.7 mg, 481 μmol, 34.2 μL, 0.800 eq) was added to the reaction mixture at 0 °C. The mixture was stirred at 25 °C for 10 min. LCMS showed trans-4-(3-(2-((4-aminocyclohexyl)amino)-5-fluoropyrimidin-4-yl)phenyl)morpholin-3-one (166-6) was remaining little and the desired mass was detected. Quench AcCl with adding water (1 mL) to the reaction mixture. The reaction mixture was concentrated under reduced pressure. The residue was purified by prep-HPLC (column: Phenomenex luna C18 100*40 mm*5 um; mobile phase: [H₂O (0.2% FA)-ACN]; gradient: 10%-50% B over 8.0 min) to give the title compound (Compound 267) (90.9 mg, 208 μmol, 34.6% yield, 97.9% purity) as a pale yellow solid. LCMS (ES⁺): m/z 428.2 [M+H]⁺.

### Example 167. Synthesis of trans-4-((5-fluoro-4-(3-(3-oxomorpholino)phenyl)pyrimidin-2-yl)amino)cyclohexane-1-carboxamide (Compound 268)

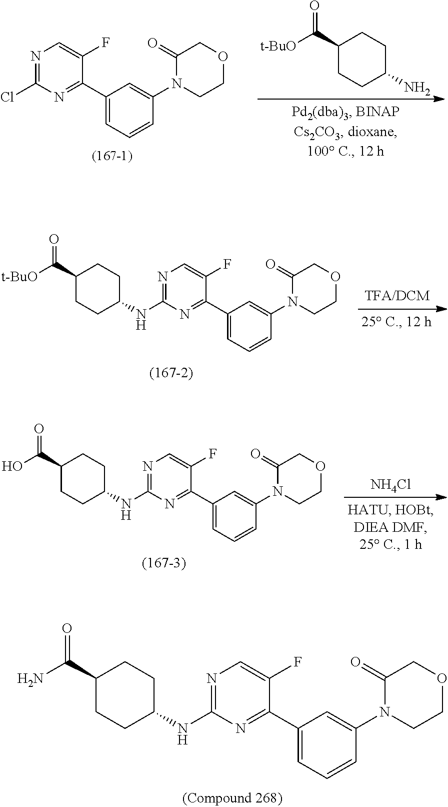 [SMILES: O=C1COCCN1c1cccc(-c2nc(Cl)ncc2F)c1 ; CC(C)(C)OC(=O)[C@H]1CC[C@H](N)CC1 ; CC(C)(C)OC(=O)[C@H]1CC[C@H](Nc2ncc(F)c(-c3cccc(N4CCOCC4=O)c3)n2)CC1 ; O=C1COCCN1c1cccc(-c2nc(N[C@H]3CC[C@H](C(=O)O)CC3)ncc2F)c1 ; "NH4Cl" ; NC(=O)[C@H]1CC[C@H](Nc2ncc(F)c(-c3cccc(N4CCOCC4=O)c3)n2)CC1]

Synthesis of trans-tert-butyl-4-((5-fluoro-4-(3-(3-oxomorpholino)phenyl)pyrimidin-2-yl)amino)cyclohexane-1-carboxylate (167-2). To a solution of 4-(3-(2-chloro-5-fluoropyrimidin-4-yl)phenyl)morpholin-3-one (167-1) (1.00 g, 3.25 mmol, 1.00 eq) and trans-tert-butyl 4-aminocyclohexanecarboxylate (1.30 g, 6.50 mmol, 2.00 eq) in dioxane (10.0 mL) were added BINAP (202 mg, 325 μmol, 0.100 eq), Cs₂CO₃ (3.18 g, 9.75 mmol, 3.00 eq) and Pd₂(dba)₃ (298 mg, 325 μmol, 0.100 eq) at 25 °C under N₂ atmosphere. The mixture was stirred at 100 °C for 12 h under N₂. LCMS showed 4-(3-(2-chloro-5-fluoropyrimidin-4-yl)phenyl)morpholin-3-one (167-1) was consumed completely and the desired mass was detected. The reaction mixture was filtered and the filtrate was concentrated under reduced pressure to give a residue. The residue was purified by MPLC (SiO₂, Petroleum ether:Ethyl acetate=1:0 to 1:2) to give the title compound (167-2) (700 mg, 1.24 mmol, 38.3% yield, 83.6% purity) as a yellow solid. LCMS (ES⁺): m/z 471.2 [M+H]⁺.

Synthesis of trans-4-((5-fluoro-4-(3-(3-oxomorpholino)phenyl)pyrimidin-2-yl)amino)cyclohexane-1-carboxylic acid (167-3). To a solution of trans-tert-butyl-4-((5-fluoro-4-(3-(3-oxomorpholino)phenyl)pyrimidin-2-yl)amino)cyclohexane-1-carboxylate (167-2) (300 mg, 638 μmol, 1.00 eq) in DCM (3 mL) was added TFA (921 mg, 8.08 mmol, 0.600 mL, 12.7 eq) at 25 °C. The mixture was stirred at 25 °C for 12 h. LCMS showed trans-tert-butyl-4-((5-fluoro-4-(3-(3-oxomorpholino)phenyl)pyrimidin-2-yl)amino)cyclohexane-1-carboxylate (167-2) was consumed completely and the desired mass was detected. The reaction mixture was concentrated under reduced pressure to give the title compound (167-3) (300 mg, crude, TFA) as a yellow oil. LCMS (ES⁺): m/z 415.1 [M+H]⁺.

Synthesis of trans-4-((5-fluoro-4-(3-(3-oxomorpholino)phenyl)pyrimidin-2-yl)amino)cyclohexane-1-carboxamide (Compound 268). To a solution of trans-4-((5-fluoro-4-(3-(3-oxomorpholino)phenyl)pyrimidin-2-yl)amino)cyclohexane-1-carboxylic acid (167-3) (300 mg, 568 μmol, 1.00 eq, TFA) and NH₄Cl (137 mg, 2.55 mmol, 4.50 eq) in DMF (3.00 mL) were added HOBt (38.4 mg, 284 μmol, 0.500 eq), HATU (259 mg, 681 μmol, 1.20 eq) and DIEA (367 mg, 2.84 mmol, 494 μL, 5.00 eq) at 25 °C. The mixture was stirred at 25 °C for 1 h. LCMS showed trans-4-((5-fluoro-4-(3-(3-oxomorpholino)phenyl)pyrimidin-2-yl)amino)cyclohexane-1-carboxylic acid (167-3) was consumed completely and the desired mass was detected. The reaction mixture was filtered and the filtrate was concentrated under reduced pressure to give a residue. The residue was purified by prep-HPLC (column: Phenomenex luna C18 100*40 mm*5 um; mobile phase: [H₂O (0.2% FA)-ACN]; gradient: 5%-45% B over 8.0 min) to give the title compound (Compound 268) (74.3 mg, 164 μmol, 29.0% yield, 91.5% purity) as a bright yellow solid. LCMS (ES⁺): m/z 414.2 [M+H]⁺.

### Example 168. Synthesis of cis-3-((5-fluoro-4-(3-(3-oxomorpholino)phenyl)pyrimidin-2-yl)amino)cyclohexane-1-carboxamide (Compound 269)

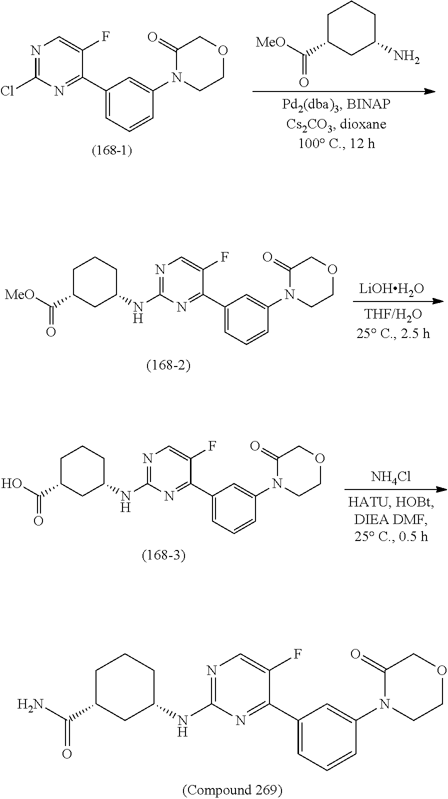

Synthesis of cis-methyl 3-((5-fluoro-4-(3-(3-oxomorpholino)phenyl)pyrimidin-2-yl)amino)cyclohexane-1-carboxylate (168-2). To a solution of cis-methyl 3-aminocyclohexane-1-carboxylate (1.02 g, 6.50 mmol, 2.00 eq) in dioxane (20 mL) were added 4-(3-(2-chloro-5-fluoropyrimidin-4-yl)phenyl)morpholin-3-one (168-1) (1.00 g, 3.25 mmol, 1.00 eq), Cs₂CO₃ (3.18 g, 9.75 mmol, 3.00 eq), BINAP (202 mg, 325 μmol, 0.100 eq) and Pd₂(dba)₃ (298 mg, 325 μmol, 0.100 eq) at 25 °C under N₂ atmosphere. The mixture was stirred at 100 °C for 12 h under N₂. LC-MS showed 4-(3-(2-chloro-5-fluoropyrimidin-4-yl)phenyl)morpholin-3-one (168-1) remained little, and the desired mass was detected. Water (20 mL) was added to the mixture at 25 °C. The reaction mixture was extracted with EtOAc (10 mL*5). The organic layers were dried over anhydrous sodium sulfate, filtered and the filtrate was concentrated under reduced to give a residue. The residue was purified by column chromatography (SiO₂, Petroleum ether:Tetrahydrofuran=4:1 to 3:1) to give the title compound (168-2) (600 mg, 1.40 mmol, 43.1% yield) as a yellow oil. LCMS (ES⁺): m/z 429.2 [M+H]⁺.

Synthesis of cis-3-((5-fluoro-4-(3-(3-oxomorpholino)phenyl)pyrimidin-2-yl)amino)cyclohexane-1-carboxylic acid (168-3). To a solution of cis-methyl 3-((5-fluoro-4-(3-(3-oxomorpholino)phenyl)pyrimidin-2-yl)amino)cyclohexane-1-carboxylate (168-2) (300 mg, 700 μmol, 1.00 eq) in THF (2.5 mL) and H₂O (1 mL) was added LiOH·H₂O (88.0 mg, 2.10 mmol, 3.00 eq) at 25 °C. The mixture was stirred at 25 °C for 2.5 h. LC-MS showed cis-methyl 3-((5-fluoro-4-(3-(3-oxomorpholino)phenyl)pyrimidin-2-yl)amino)cyclohexane-1-carboxylate (168-2) was consumed completely, and the desired mass was detected. Water (2 mL) was added to the mixture at 25 °C. The reaction mixture was washed with EtOAC (4 mL). HCl (1 M) was added to the aqueous phase at 25 °C to adjust pH 2 to 3. The above aqueous phase was extracted with EtOAC (4 mL*5). The organic layers were dried over anhydrous sodium sulfate, filtered and the filtrate was concentrated under reduced to give the title compound (168-3) (95.0 mg, crude) as a yellow oil. LCMS (ES⁺): m/z 415.1 [M+H]⁺.

Synthesis of cis-3-((5-fluoro-4-(3-(3-oxomorpholino)phenyl)pyrimidin-2-yl)amino)cyclohexane-1-carboxamide (Compound 269). To a solution of cis-3-((5-fluoro-4-(3-(3-oxomorpholino)phenyl)pyrimidin-2-yl)amino)cyclohexane-1-carboxylic acid (168-3) (95.0 mg, 229 μmol, 1.00 eq) in DMF (2 mL) were added HATU (105 mg, 275 μmol, 1.20 eq), HOBt (15.5 mg, 115 μmol, 0.500 eq), DIEA (148 mg, 1.15 mmol, 120 μL, 5.00 eq) and NH₄Cl (61.3 mg, 1.15 mmol, 5.00 eq) at 25 °C. The mixture was stirred at 25 °C for 0.5 h. LC-MS showed cis-3-((5-fluoro-4-(3-(3-oxomorpholino)phenyl)pyrimidin-2-yl)amino)cyclohexane-1-carboxylic acid (168-3) was remaining little, and the desired mass was detected. The reaction mixture was filtered and the filtrate was concentrated under reduced pressure to give a residue. The residue was purified by prep-HPLC (column: Phenomenex luna C18 100*40 mm*5 um; mobile phase: [1H₂O (0.2% FA)-ACN]; gradient: 1%-40% B over 8.0 min) to give the title compound (Compound 269) (143 mg, 334 μmol, 96.8% purity) as a pale yellow solid. LCMS (ES⁺): m/z 414.2 [M+H]⁺.

### Example 169. Synthesis of 1-[3-[2-[(1-acetyl-4-piperidyl)amino]-5-fluoro-pyrimidin-4-yl]phenyl]pyrrolidin-2-one (Compound 222)

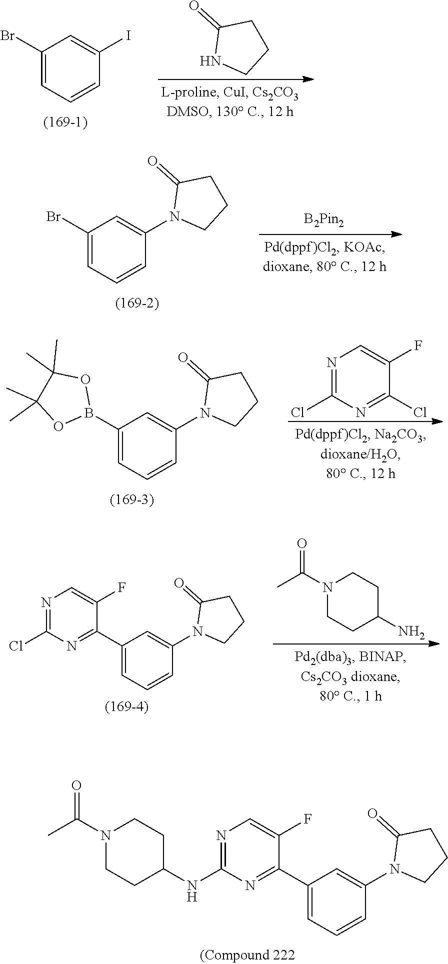

Synthesis of 1-(3-bromophenyl)pyrrolidin-2-one (169-2). To a solution of 1-bromo-3-iodo-benzene (169-1) (10.0 g, 35.3 mmol, 4.51 mL, 1.00 eq) and pyrrolidin-2-one (4.51 g, 53.0 mmol, 4.07 mL, 1.50 eq) in DMSO (200 mL) was added CuI (673 mg, 3.53 mmol, 0.100 eq) and L-proline (406 mg, 3.53 mmol, 0.100 eq) and Cs₂CO₃ (34.5 g, 106 mmol, 3.00 eq) at 25 °C. The mixture was stirred at 130 °C for 12 h. LC-MS showed desired mass was detected. The reaction mixture was concentrated under reduced pressure. The residue was diluted with water 200 mL and extracted with Ethyl acetate (200 mL*2). The combined organic layers were washed with brine (200 mL*2), dried over Na₂SO₄, filtered and concentrated under reduced pressure. The residue was purified by column chromatography (SiO₂, Petroleum ether:Ethyl acetate=3:1 to 2:1) to give the title compound (169-2) (6 g, 23.8 mmol, 67.4% yield, 95.3% purity) as a brown solid. LCMS (ES⁺): m/z 240.0 [M+H]⁺.

Synthesis of 1-[3-(4,4,5,5-tetramethyl-1,3,2-dioxaborolan-2-yl)phenyl]pyrrolidin-2-one (169-3). To a solution of 1-(3-bromophenyl)pyrrolidin-2-one (169-2) (3 g, 12.4 mmol, 1.00 eq) in dioxane (80 mL) was added Pd(dppf)Cl₂ (457 mg, 624 μmol, 0.05 eq) and Pin₂B₂ (4.76 g, 18.7 mmol, 1.50 eq) and KOAc (3.68 g, 37.4 mmol, 3.00 eq) at 25 °C. The mixture was stirred at 80 °C for 12 h. LC-MS showed desired mass was detected. The reaction mixture was concentrated under reduced pressure. The residue was diluted with water 80 mL and extracted with Ethyl acetate (80 mL*2). The combined organic layers were washed with brine (80 mL*2), dried over Na₂SO₄, filtered and concentrated under reduced pressure. The residue was purified by column chromatography (SiO₂, Petroleum ether:Ethyl acetate=3:1 to 2:1) to give the title compound (169-3) (3 g, 8.84 mmol, 70.8% yield, 84.6% purity) as a white solid. LCMS (ES⁺): m/z 288.2 [M+H]⁺.

Synthesis of 1-[3-(2-chloro-5-fluoro-pyrimidin-4-yl)phenyl]pyrrolidin-2-one (169-4). To a solution of 1-[3-(4,4,5,5-tetramethyl-1,3,2-dioxaborolan-2-yl)phenyl]pyrrolidin-2-one (169-3) (1.00 g, 3.48 mmol, 1.00 eq) and 2,4-dichloro-5-fluoro-pyrimidine (697 mg, 4.18 mmol, 1.20 eq) in dioxane (30 mL) and H₂O (5 mL) was added Pd(dppf)Cl₂ (254 mg, 348 μmol, 0.100 eq) and Na₂CO₃ (1.11 g, 10.4 mmol, 3.00 eq) at 25 °C. The mixture was stirred at 80 °C for 12 h. LC-MS showed the desired mass was detected. The reaction mixture was concentrated under reduced pressure. The residue was diluted with water 40 mL and extracted with Ethyl acetate (40 mL*2). The combined organic layers were washed with brine (40 mL*2), dried over Na₂SO₄, filtered and concentrated under reduced pressure. The residue was purified by prep-HPLC (column: Phenomenex luna C18 100*40 mm*5 um; mobile phase: [H₂O (0.2% FA)-ACN]; gradient: 1%-40% B over 8.0 min) to give the title compound (169-4) (500 mg, 1.60 mmol, 45.98% yield, 93.41% purity) as a white solid. LCMS (ES⁺): m/z 292.0 [M+H]⁺.

Synthesis of 1-[3-[2-[(1-acetyl-4-piperidyl)amino]-5-fluoro-pyrimidin-4-yl]phenyl]pyrrolidin-2-one (Compound 222). To a solution of 1-[3-(2-chloro-5-fluoro-pyrimidin-4-yl)phenyl]pyrrolidin-2-one (169-4) (200 mg, 686 μmol, 1.00 eq) and 1-(4-amino-1-piperidyl)ethanone (117 mg, 823 μmol, 1.20 eq) in dioxane (3 mL) was added Pd₂(dba)₃ (62.8 mg, 68.6 μmol, 0.100 eq) and BINAP (42.7 mg, 68.6 μmol, 0.100 eq) and Cs₂CO₃ (670 mg, 2.06 mmol, 3.00 eq) at 25 °C. The mixture was stirred at 80 °C for 1 h under N₂. LC-MS showed 1-[3-(2-chloro-5-fluoro-pyrimidin-4-yl)phenyl]pyrrolidin-2-one (169-4) was consumed completely and the desired mass was detected. The reaction mixture was diluted with water 3 mL and extracted with EtOAc (3 mL*3). The combined organic layers were washed with brine (3 mL*3), dried over Na₂SO₄, filtered and concentrated under reduced pressure. The residue was purified by prep-HPLC (column: Phenomenex Luna C18 100*30 mm*3 um; mobile phase: [H₂O (0.2% FA)-ACN]; gradient: 10%-45% B over 8.0 min) to give the title compound (Compound 222) (113 mg, 271 μmol, 39.5% yield, 95.2% purity) as a white solid. LCMS (ES⁺): m/z 398.2 [M+H]⁺.

### Example 170. Synthesis of trans-N-(4-((5-fluoro-4-(3-(2-oxopyrrolidin-1-yl)phenyl)pyrimidin-2-yl)amino)cyclohexyl)acetamide (Compound 248)

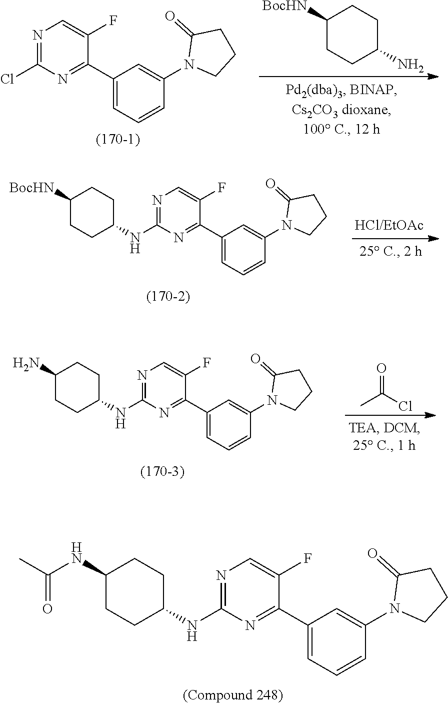

Synthesis of trans-tert-butyl (4-((5-fluoro-4-(3-(2-oxopyrrolidin-1-yl)phenyl)pyrimidin-2-yl)amino)cyclohexyl)carbamate (170-2). A mixture of 1-[3-(2-chloro-5-fluoro-pyrimidin-4-yl)phenyl]pyrrolidin-2-one (170-1) (500 mg, 1.71 mmol, 1.00 eq), trans-tert-butyl (4-aminocyclohexyl)carbamate (550 mg, 2.57 mmol, 1.50 eq), Pd₂(dba)₃ (156 mg, 171 μmol, 0.100 eq), BINAP (106 mg, 171 μmol, 0.100 eq) and Cs₂CO₃ (1.68 g, 5.14 mmol, 3.00 eq) in dioxane (10 mL) at 25 °C was degassed and purged with N₂ for 3 times, and then the mixture was stirred at 100 °C for 12 h under N₂ atmosphere. LC-MS showed the desired mass was detected. The reaction mixture was diluted with water 8 mL and extracted with EtOAc (15 mL*3), filtered and concentrated under reduced pressure. The residue was purified by prep-TLC (SiO₂, Petroleum ether:Ethyl acetate=1:0 to 1:1) to give the title compound (170-2) (610 mg, 1.04 mmol, 60.8% yield, 80.2% purity) as a yellow solid. LCMS (ES⁺): m/z 470.3 [M+H]⁺.

Synthesis of trans-1-(3-(2-((-4-aminocyclohexyl)amino)-5-fluoropyrimidin-4-yl)phenyl)pyrrolidin-2-one (170-3). trans-tert-butyl (4-((5-fluoro-4-(3-(2-oxopyrrolidin-1-yl)phenyl)pyrimidin-2-yl)amino)cyclohexyl)carbamate (170-2) (600 mg, 1.28 mmol, 1.00 eq) in HCl/EtOAc (4 M, 319 μL, 1.00 eq) at 25 °C. The mixture was stirred at 25 °C for 2 h. LC-MS showed the desired mass was detected. The reaction mixture was concentrated under reduced pressure to give the title compound (170-3) (450 mg, crude, HCl) as a yellow oil. LCMS (ES⁺): m/z 370.2 [M+H]⁺.

Synthesis of trans-N-(4-((5-fluoro-4-(3-(2-oxopyrrolidin-1-yl)phenyl)pyrimidin-2-yl)amino)cyclohexyl)acetamide (Compound 248). To a solution of trans-1-(3-(2-((-4-aminocyclohexyl)amino)-5-fluoropyrimidin-4-yl)phenyl)pyrrolidin-2-one (170-3) (150 mg, 406 μmol, 1.00 eq) in DCM (3 mL) was added TEA (123 mg, 1.22 mmol, 169 μL, 3.00 eq) and acetyl chloride (35.0 mg, 446 μmol, 31.7 μL, 1.10 eq) at 25 °C. The mixture was stirred at 25 °C for 1 h. LC-MS showed the desired mass was detected. The reaction mixture was quenched by addition water 2 mL at 0 °C, and extracted with EtOAc (2 mL*3). The combined organic layers were washed with brine (2 mL*3), dried over Na₂SO₄, filtered and concentrated under reduced pressure. The residue was purified by prep-HPLC (column: Phenomenex Luna C18 75*30 mm*3 um; mobile phase: [H₂O (0.2% FA)-ACN]; gradient: 20%-50% B over 8.0 min) to give the title compound (Compound 248) (43.4 mg, 103 μmol, 28.3% yield, 98.0% purity) as a white solid. LCMS (ES⁺): m/z 412.2 [M+H]+.

### Example 171. Synthesis of trans-4-((5-fluoro-4-(3-(2-oxopyrrolidin-1-yl)phenyl)pyrimidin-2-yl)amino)cyclohexane-1-carboxamide (Compound 249)

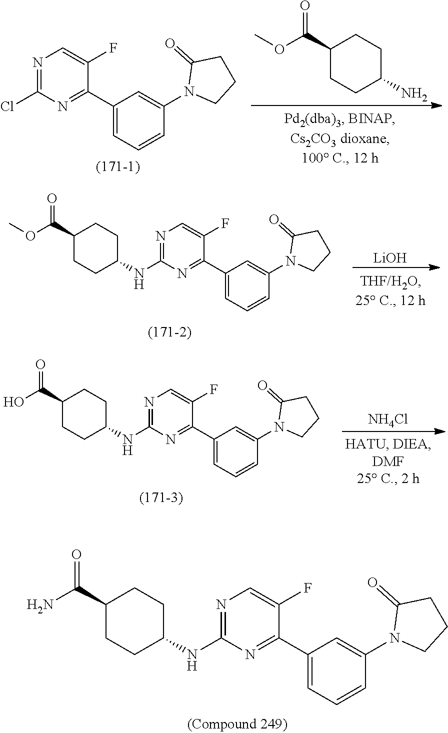

Synthesis of trans-methyl 4-((5-fluoro-4-(3-(2-oxopyrrolidin-1-yl)phenyl)pyrimidin-2-yl)amino)cyclohexane-1-carboxylate (171-2). A mixture of 1-[3-(2-chloro-5-fluoro-pyrimidin-4-yl)phenyl]pyrrolidin-2-one (171-1) (800 mg, 2.74 mmol, 1.00 eq), trans-methyl 4-aminocyclohexanecarboxylate (517 mg, 3.29 mmol, 1.20 eq), BINAP (341 mg, 548 μmol, 0.200 eq), Pd₂(dba)₃ (502 mg, 548 μmol, 0.200 eq) and Cs₂CO₃ (2.68 g, 8.23 mmol, 3.00 eq) in dioxane (20 mL) at 25 °C was degassed and purged with N₂ for 3 times, and then the mixture was stirred at 100 °C for 12 h under N₂ atmosphere. LC-MS showed 1-[3-(2-chloro-5-fluoro-pyrimidin-4-yl)phenyl]pyrrolidin-2-one (171-1) was consumed completely and the desired mass was detected. The reaction diluted with H₂O 30 mL and combined organic layers were extracted with EtOAc (50 mL*3), and concentrated under reduced pressure. The residue was purified by column chromatography (SiO₂, Petroleum ether:Ethyl acetate=1:0 to 0:1) to give the title compound (171-2) (800 mg, 1.76 mmol, 64.1% yield, 90.6% purity) as a yellow solid LCMS (ES⁺): m/z 413.2 [M+H]⁺.

Synthesis of trans-4-((5-fluoro-4-(3-(2-oxopyrrolidin-1-yl)phenyl)pyrimidin-2-yl)amino)cyclohexane-1-carboxylic acid (171-3). To a solution of trans-methyl 4-((5-fluoro-4-(3-(2-oxopyrrolidin-1-yl)phenyl)pyrimidin-2-yl)amino)cyclohexane-1-carboxylate (171-2) (800 mg, 1.94 mmol, 1.00 eq) in THF (50 mL) and H₂O (10 mL) was added LiOH (139 mg, 5.82 mmol, 3.00 eq) at 25 °C. The mixture was stirred at 25 °C for 12 h. LC-MS showed trans-methyl 4-((5-fluoro-4-(3-(2-oxopyrrolidin-1-yl)phenyl)pyrimidin-2-yl)amino)cyclohexane-1-carboxylate (171-2) was consumed completely and the desired mass was detected. The reaction mixture was filtered and concentrated under reduced pressure. Drop AcOH to adjust PH to 5. Combined organic layers were extracted with EtOAc (50 mL*3), and concentrated under reduced pressure to give the title compound (171-3) (500 mg, crude) as a brown solid. LCMS (ES⁺): m/z 399.2 [M+H]⁺.

Synthesis of trans-4-((5-fluoro-4-(3-(2-oxopyrrolidin-1-yl)phenyl)pyrimidin-2-yl)amino)cyclohexane-1-carboxamide (Compound 249). To a solution of trans-4-((5-fluoro-4-(3-(2-oxopyrrolidin-1-yl)phenyl)pyrimidin-2-yl)amino)cyclohexane-1-carboxylic acid (171-3) (100 mg, 250 μmol, 1.00 eq) in DMF (3 mL) was added HATU (95.4 mg, 250 μmol, 1.00 eq), NH₄Cl (16.1 mg, 301 μmol, 1.20 eq) and DIEA (97.3 mg, 752 μmol, 131 μL, 3.00 eq) at 25 °C. The mixture was stirred at 25 °C for 2 h. LC-MS showed trans-4-((5-fluoro-4-(3-(2-oxopyrrolidin-1-yl)phenyl)pyrimidin-2-yl)amino)cyclohexane-1-carboxylic acid (171-3) was consumed completely and the desired mass was detected. The reaction mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 15%-45% B over 8.0 min) to give the title compound (Compound 249) (37.1 mg, 90.9 μmol, 36.2% yield, 97.4% purity) as an off-white solid. LCMS (ES⁺): m/z 398.2 [M+H]+.

### Example 172. Synthesis of 3-(3-(2-((1-acetylpiperidin-4-yl)amino)-5-fluoropyrimidin-4-yl)phenyl)oxazolidin-2-one (Compound 226)

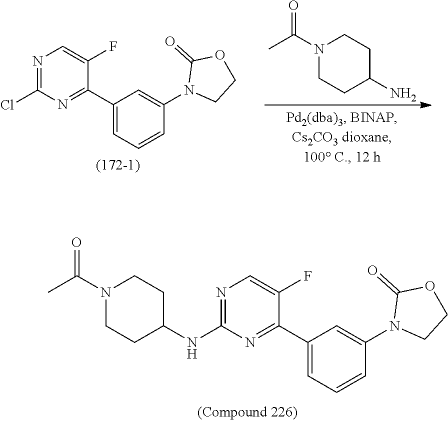

Synthesis of 3-(3-(2-((1-acetylpiperidin-4-yl)amino)-5-fluoropyrimidin-4-yl)phenyl)oxazolidin-2-one (Compound 226). A mixture of 3-(3-(2-chloro-5-fluoropyrimidin-4-yl)phenyl)oxazolidin-2-one (172-1) (150 mg, 510 μmol, 1.00 eq), 1-(4-amino-1-piperidyl)ethanone (72.6 mg, 510 μmol, 1.00 eq), BINAP (31.8 mg, 51.0 μmol, 0.100 eq), Pd₂(dba)₃ (46.7 mg, 51.0 μmol, 0.100 eq) and Cs₂CO₃ (832 mg, 2.55 mmol, 5.00 eq) in 1,4-dioxane (2 mL) was degassed and purged with N₂ at 20 °C for 3 times, and then the mixture was stirred at 100 °C for 12 h under N₂ atmosphere. LC-MS showed the desired mass was detected. The reaction mixture was quenched by addition H₂O 10 mL, and then diluted with H₂O 10 mL and extracted with EtOAc (10 mL*3). The combined organic layers were washed with sat.aq. NaCl (10 mL), dried over Na₂SO₄, filtered and concentrated under reduced pressure. The residue was purified by prep-HPLC (column: Phenomenex Gemini-NX 100*30 mm*3 um; mobile phase: [H₂O (10 mM NH₄HCO₃)-ACN]; gradient: 12%-42% B over 8.0 min) to give the title compound (Compound 226) (27.8 mg, 69.6 μmol, 13.6% yield, 100% purity) as a white solid. LCMS (ES⁺): m/z 400.2 [M+H]⁺.

### Example 173. Synthesis of trans-N-(4-((5-fluoro-4-(3-(2-oxooxazolidin-3-yl)phenyl)pyrimidin-2-yl)amino)cyclohexyl)acetamide (Compound 233)

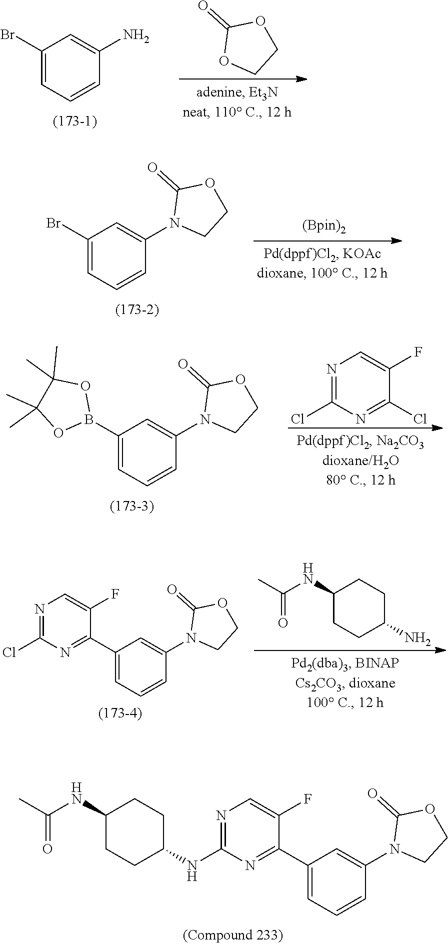

Synthesis of 3-(3-bromophenyl)oxazolidin-2-one (173-2). A solution of 3-bromoaniline (173-1) (7.65 g, 44.5 mmol, 4.84 mL, 1.00 eq), 1,3-dioxolan-2-one (23.5 g, 267 mmol, 17.8 mL, 6.00 eq), adenine (301 mg, 2.22 mmol, 0.050 eq) and Et₃N (2.25 g, 22.2 mmol, 3.10 mL, 0.500 eq) was stirred at 110 °C for 12 h as a neat reaction. LCMS showed the desired mass was detected. H₂O (100 mL) was added to the solution, then the mixture was extracted with DCM (100 mL*3), the combined organic layers were dried over Na₂SO₄, filtered and the filtrate was concentrated under reduced pressure to give the crude product. The crude product was purified by M PLC (SiO₂, Petroleum ether/Ethyl acetate=1/0 to 1/1) to give the title compound (173-2) (10.0 g, 41.3 mmol, 92.9% yield) as a white solid. LCMS (ES⁺): m/z 242.0 [M+H]⁺.

Synthesis of 3-(3-(4,4,5,5-tetramethyl-1,3,2-dioxaborolan-2-yl)phenyl)oxazolidin-2-one (173-3). To a solution of 3-(3-bromophenyl)oxazolidin-2-one (173-2) (7.00 g, 28.9 mmol, 1.00 eq) and 4,4,5,5-tetramethyl-2-(4,4,5,5-tetramethyl-1,3,2-dioxaborolan-2-yl)-1,3,2-dioxaborolane (9.55 g, 37.6 mmol, 1.30 eq) in dioxane (200 mL) were added Pd(dppf)Cl₂ (2.12 g, 2.89 mmol, 0.100 eq) and KOAc (8.51 g, 86.8 mmol, 3.00 eq) at 25 °C under N₂. The mixture was stirred at 100 °C for 12 h under N₂. LCMS showed that 3-(3-bromophenyl)oxazolidin-2-one (173-2) was consumed completely and the desired mass was detected. H₂O (200 mL) was added to the solution, then the mixture was extracted with EtOAc (200 mL*3), the combined organic layers were dried over Na₂SO₄, filtered and the filtrate was concentrated under reduced pressure to give the crude product. The crude product was purified by MPLC (SiO₂, Petroleum ether/Ethyl acetate=1/0 to 1/1) to give the title compound (173-3) (8.00 g, 27.7 mmol, 95.7% yield) as a yellow solid, LCMS (ES⁺): m/z 290.0 [M+H]⁺.

Synthesis of 3-(3-(2-chloro-5-fluoropyrimidin-4-yl)phenyl)oxazolidin-2-one (173-4). To a solution of 3-(3-(4,4,5,5-tetramethyl-1,3,2-dioxaborolan-2-yl)phenyl)oxazolidin-2-one (173-3) (8.00 g, 27.7 mmol, 1.00 eq) and 2,4-dichloro-5-fluoropyrimidine (5.54 g, 33.2 mmol, 1.20 eq) in dioxane (80 mL) and H₂O (15 mL) were added Na₂CO₃ (5.87 g, 55.3 mmol, 2.00 eq) and Pd(dppf)Cl₂ (2.02 g, 2.77 mmol, 0.100 eq) at 25 °C under N₂. The mixture was stirred at 80 °C for 12 h under N₂. LCMS showed that 3-(3-(4,4,5,5-tetramethyl-1,3,2-dioxaborolan-2-yl)phenyl)oxazolidin-2-one (173-3) was consumed completely the desired mass was detected, H₂O (100 mL) was added to the solution, then the mixture was extracted with EtOAc (100 mL*3), the combined organic layers were dried over Na₂SO₄, filtered and the filtrate was concentrated under reduced pressure to give the crude product. The crude product was purified by M PLC (SiO₂, Petroleum ether/Ethyl acetate=1/0 to 1/1) to give the title compound (173-4) (7.30 g, 24.9 mmol, 89.8% yield) as a brown solid. LCMS (ES⁺): m/z 294.0 [M+H]⁺.

Synthesis of trans-N-(4-((5-fluoro-4-(3-(2-oxooxazolidin-3-yl)phenyl)pyrimidin-2-yl)amino)cyclohexyl)acetamide (Compound 233). To a solution of 3-(3-(2-chloro-5-fluoropyrimidin-4-yl)phenyl)oxazolidin-2-one (173-4) (250 mg, 851 μmol, 1.00 eq) and trans-N-(4-aminocyclohexyl)acetamide (266 mg, 1.70 mmol, 2.00 eq) in dioxane (10 mL) were added Pd₂(dba)₃ (78.0 mg, 85.1 μmol, 0.100 eq), BINAP (53.0 mg, 85.1 umol, 0.100 eq) and Cs₂CO₃ (555 mg, 1.70 mmol, 2.00 eq) at 25 °C under N₂. After addition, the mixture was stirred at 100 °C for 12 h under N₂. LC-MS showed that the desired mass was detected. The mixture was filtered and the filtrate was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 10%-40% B over 8.0 min) to give the title compound (Compound 233) (20.0 mg, 47.6 μmol, 5.59% yield, 98.40% purity) as a white solid. LCMS (ES⁺): m/z 414.2 [M+H]⁺.

### Example 174. Synthesis of trans-4-((5-fluoro-4-(3-(2-oxooxazolidin-3-yl)phenyl)pyrimidin-2-yl)amino)cyclohexane-1-carboxamide (Compound 245)

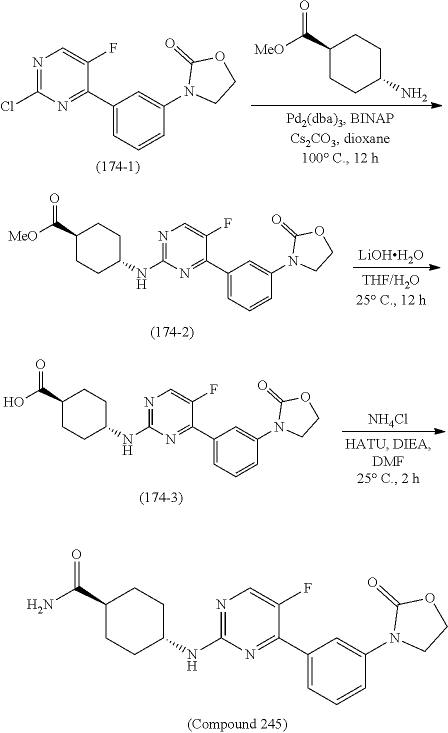

Synthesis of trans-methyl-4-((5-fluoro-4-(3-(2-oxooxazolidin-3-yl)phenyl)pyrimidin-2-yl)amino)cyclohexane-1-carboxylate (174-2). To a solution of 3-(3-(2-chloro-5-fluoropyrimidin-4-yl)phenyl)oxazolidin-2-one (174-1) (700 mg, 2.38 mmol, 1.00 eq) and trans-methyl-4-aminocyclohexane-1-carboxylate (562 mg, 3.58 mmol, 1.50 eq) in dioxane (7 mL) were added Pd₂(dba)₃ (218 mg, 238 μmol, 0.100 eq), BINAP (148 mg, 238 μmol, 0.100 eq) and Cs₂CO₃ (1.55 g, 4.77 mmol, 2.00 eq) at 25 °C under N₂ atmosphere. After addition, the mixture was stirred at 100 °C for 12 h under N₂. LC-MS showed that the desired mass was detected. H₂O (15 mL) was added to the solution, then the mixture was extracted with EtOAc (15 mL*3), the combined organic layers were dried over Na₂SO₄, filtered and the filtrate was concentrated under reduced pressure to give the crude product. The crude product was purified by MPLC (SiO₂, Petroleum ether/Ethyl acetate=1/0 to 1/1) to give the title compound (174-2) (320 mg, 772 μmol, 32.4% yield) as a yellow solid. LCMS (ES⁺): m/z 415.2 [M+H]⁺.

Synthesis of trans-4-((5-fluoro-4-(3-(2-oxooxazolidin-3-yl)phenyl)pyrimidin-2-yl)amino)cyclohexane-1-carboxylic acid (174-3). To a solution of trans-methyl-4-((5-fluoro-4-(3-(2-oxooxazolidin-3-yl)phenyl)pyrimidin-2-yl)amino)cyclohexane-1-carboxylate (174-2) (300 mg, 724 μmol, 1.00 eq) in THF (6 mL) and H₂O (2 mL) was added LiOH·H₂O (43.3 mg, 1.81 mmol, 2.50 eq) at 25 °C. After addition, the mixture was stirred at 25 °C for 12 h. LC-MS showed that trans-methyl-4-((5-fluoro-4-(3-(2-oxooxazolidin-3-yl)phenyl)pyrimidin-2-yl)amino)cyclohexane-1-carboxylate (174-2) was consumed completely and the desired mass was detected. The reaction was concentrated under reduced pressure to remove THF. Then to the remaining aqueous phase was added HCl (1 M) to adjust pH to 3, then solid was precipitated, filtered and the filter cake was dried under reduced pressure to give the title compound (174-3) (140 mg, crude) as a yellow solid. LCMS (ES⁺): m/z 401.2 [M+H]⁺.

Synthesis of trans-4-((5-fluoro-4-(3-(2-oxooxazolidin-3-yl)phenyl)pyrimidin-2-yl)amino)cyclohexane-1-carboxamide (Compound 245). To a solution of trans-4-((5-fluoro-4-(3-(2-oxooxazolidin-3-yl)phenyl)pyrimidin-2-yl)amino)cyclohexane-1-carboxylic acid (174-3) (130 mg, 325 μmol, 1.00 eq) in DMF (4 mL) were added HATU (185 mg, 487 μmol, 1.50 eq) and DIEA (126 mg, 974 μmol, 170 μL, 3.00 eq) at 25 °C. And then NH₄Cl (17.4 mg, 325 mol, 1.00 eq) was added to the mixture at 25 °C. The resulting mixture was stirred at 25 °C for 2 h. LC-MS showed that trans-4-((5-fluoro-4-(3-(2-oxooxazolidin-3-yl)phenyl)pyrimidin-2-yl)amino)cyclohexane-1-carboxylic acid (174-3) was consumed completely and the desired mass was detected. The mixture was concentrated under reduced pressure to give a residue. The residue was purified by prep-HPLC (column: Phenomenex Luna C18 75*30 mm*3 um; mobile phase: [H₂O (0.2% FA)-ACN]; gradient: 25%-55% B over 8.0 min) to give the title compound (Compound 245) (40.8 mg, 102 μmol, 31.5% yield, 100.00% purity) as a white solid. LCMS (ES⁺): m/z 400.1 [M+H]⁺.

### Example 175. Synthesis of (4R)-1-[3-[2-[(1-acetyl-4-piperidyl)amino]-5-fluoro-pyrimidin-4-yl]phenyl]-4-hydroxy-pyrrolidin-2-one (Compound 237)

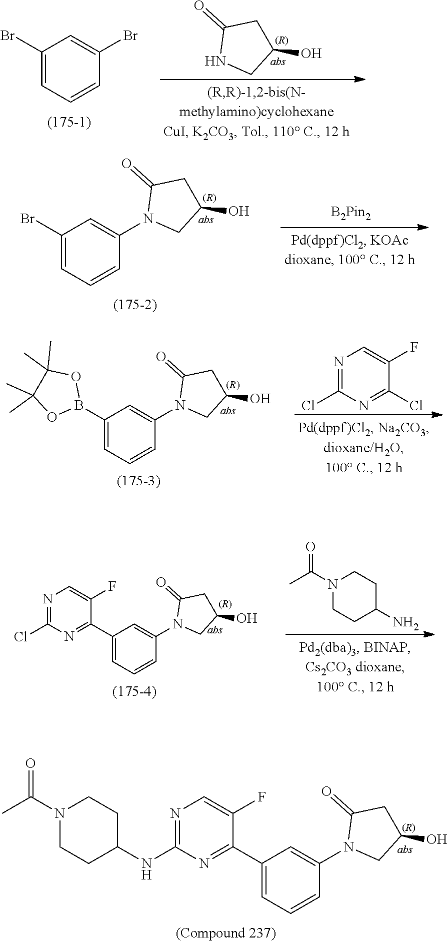

Synthesis of (4R)-1-(3-bromophenyl)-4-hydroxypyrrolidin-2-one (175-2). A mixture of 1,3-dibromobenzene (175-1) (46.6 g, 197 mmol, 23.8 mL, 2.00 eq), (4R)-4-hydroxypyrrolidin-2-one (10.0 g, 98.9 mmol, 1.00 eq), CuI (3.77 g, 19.7 mmol, 0.200 eq), K₂CO₃ (27.3 g, 197 mmol, 2.00 eq) and (1R,2R)—N1,N2-dimethylcyclohexane-1,2-diamine (2.81 g, 19.7 mmol, 0.200 eq) in toluene (50 mL) at 25 °C was degassed and purged with N₂ for 3 times, and then the mixture was stirred at 110 °C for 12 h under N₂ atmosphere. LC-MS showed the desired mass was detected. The reaction mixture was diluted with water 30 mL and extracted with EtOAc (100 mL*3), filtered and concentrated under reduced pressure. The residue was purified by column chromatography (SiO₂, Petroleum ether:Ethyl acetate=1:0 to 0:1) to give the title compound (175-2) (4.00 g, 15.2 mmol, 15.4% yield, 97.7% purity) as a brown solid. LCMS (ES⁺): m/z 256.0, 258.0 [M+H]⁺.

Synthesis of (4R)-4-hydroxy-1-[3-(4,4,5,5-tetramethyl-1,3,2-dioxaborolan-2-yl)phenyl]pyrrolidin-2-one (175-3). A mixture of (4R)-1-(3-bromophenyl)-4-hydroxy-pyrrolidin-2-one (175-2) (2.00 g, 7.81 mmol, 1.00 eq), 4,4,5,5-tetramethyl-2-(4,4,5,5-tetramethyl-1,3,2-dioxaborolan-2-yl)-1,3,2-dioxaborolane (1.98 g, 7.81 mmol, 1.00 eq), Pd(dppf)Cl₂ (571 mg, 780 μmol, 0.100 eq), KOAc (2.30 g, 23.4 mmol, 3.00 eq) in dioxane (25 mL) at 25 °C was degassed and purged with N₂ for 3 times, and then the mixture was stirred at 100 °C for 12 h under N₂ atmosphere. LC-MS showed the desired mass was detected. The reaction mixture was diluted with water 10 mL and extracted with EtOAc (30 mL*3), filtered and concentrated under reduced pressure. The residue was purified by column chromatography (SiO₂, Petroleum ether:Ethyl acetate=1:0 to 0:1) to give the title compound (175-3) (1.60 g, 4.37 mmol, 56.0% yield, 82.8% purity) as a brown solid. LCMS (ES⁺): m/z 304.1 [M+H]⁺.

Synthesis of (4R)-1-[3-(2-chloro-5-fluoro-pyrimidin-4-yl)phenyl]-4-hydroxy-pyrrolidin-2-one (175-4). A mixture of (4R)-4-hydroxy-1-[3-(4,4,5,5-tetramethyl-1,3,2-dioxaborolan-2-yl)phenyl]pyrrolidin-2-one (175-3) (800 mg, 2.64 mmol, 1.00 eq), 2,4-dichloro-5-fluoro-pyrimidine (440 mg, 2.64 mmol, 1.00 eq), Pd(dppf)Cl₂ (193 mg, 263 μmol, 0.100 eq), Na₂CO₃ (839 mg, 7.92 mmol, 3.00 eq) in dioxane (9 mL) and H₂O (3 mL) at 25 °C was degassed and purged with N₂ for 3 times, and then the mixture was stirred at 100 °C for 12 h under N₂ atmosphere. LC-MS showed the desired mass was detected. The reaction mixture was diluted with water 7 mL and extracted with EtOAc (20 mL*3), filtered and concentrated under reduced pressure. The residue was purified by prep-TLC (SiO₂, Petroleum ether:Ethyl acetate=1:0 to 0:1) to give the title compound (175-4) (360 mg, 1.08 mmol, 40.7% yield, 91.9% purity) as a brown solid. LCMS (ES⁺): m/z 308.1 [M+H]⁺.

Synthesis of (4R)-1-[3-[2-[(1-acetyl-4-piperidyl)amino]-5-fluoro-pyrimidin-4-yl]phenyl]-4-hydroxy-pyrrolidin-2-one (Compound 237). A mixture of (4R)-1-[3-(2-chloro-5-fluoro-pyrimidin-4-yl)phenyl]-4-hydroxy-pyrrolidin-2-one (175-4) (300 mg, 974 μmol, 1.00 eq), 1-(4-amino-1-piperidyl)ethanone (207 mg, 1.46 mmol, 1.50 eq), Pd₂(dba)₃ (89.2 mg, 97.5 μmol, 0.100 eq), BINAP (60.7 mg, 97.5 μmol, 0.100 eq) and Cs₂CO₃ (952 mg, 2.92 mmol, 3.00 eq) in dioxane (7 mL) at 25 °C was degassed and purged with N₂ for 3 times, and then the mixture was stirred at 100 °C for 12 h under N₂ atmosphere. LC-MS showed the desired mass was detected. The reaction mixture was filtered to give a residue. The residue was purified by prep-HPLC (column: Phenomenex luna C18 100*40 mm*3 um; mobile phase: [H₂O (0.2% FA)-ACN]; gradient: 3%-43% B over 8.0 min) to give the title compound (Compound 237) (110 mg, 260 μmol, 26.7% yield, 97.9% purity) as a white solid. LCMS (ES⁺): m/z 414.2 [M+H]⁺.

### Example 176. Synthesis of (4S)-1-[3-[2-[(1-acetyl-4-piperidyl)amino]-5-fluoro-pyrimidin-4-yl]phenyl]-4-hydroxy-pyrrolidin-2-one (Compound 230)

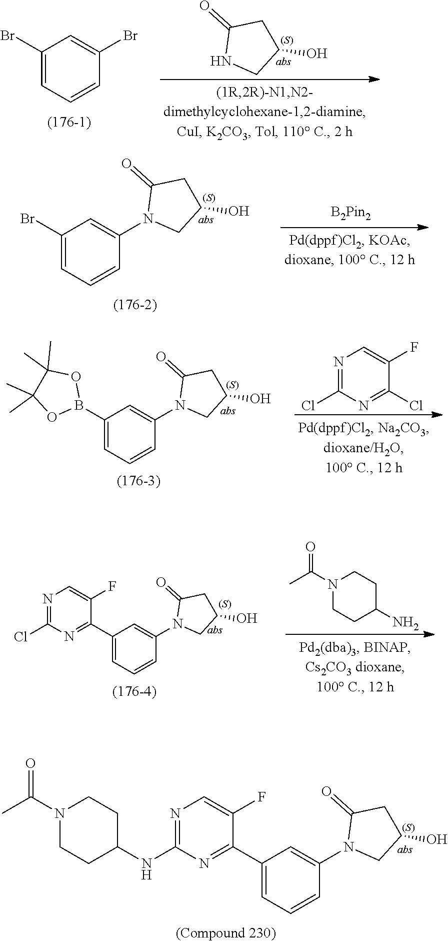

Synthesis of (4S)-1-(3-bromophenyl)-4-hydroxy-pyrrolidin-2-one (176-2). A mixture of 1,3-dibromobenzene (176-1) (18.6 g, 79.1 mmol, 9.55 mL, 2.00 eq), (4S)-4-hydroxypyrrolidin-2-one (4.00 g, 39.5 mmol, 1.00 eq), K₂CO₃ (10.9 g, 79.1 mmol, 2.00 eq), CuI (1.51 g, 7.91 mmol, 0.200 eq) and (1R,2R)—N1,N2-dimethylcyclohexane-1,2-diamine (1.13 g, 7.91 mmol, 0.200 eq) in toluene (60 mL) was degassed and purged with N₂ for 3 times at 25 °C, and then the mixture was stirred at 110 °C for 2 h under N₂ atmosphere. LC-MS showed the desired mass was detected. The reaction mixture was diluted with H₂O 40 mL and extracted with EtOAc (50 mL*3). The combined organic layers were concentrated under reduced pressure. The residue was purified by column chromatography (SiO₂, Petroleum ether:Ethyl acetate=1:0 to 1:1) to give the title compound (176-2) (1.50 g, 5.18 mmol, 13.0% yield, 88.4% purity) as a black solid. LCMS (ES⁺): m/z 256.0 [M+H]⁺.

Synthesis of (4S)-4-hydroxy-1-[3-(4,4,5,5-tetramethyl-1,3,2-dioxaborolan-2-yl)phenyl]pyrrolidin-2-one (176-3). A mixture of (4S)-1-(3-bromophenyl)-4-hydroxy-pyrrolidin-2-one (176-2) (1.40 g, 5.47 mmol, 1.00 eq), 4,4,5,5-tetramethyl-2-(4,4,5,5-tetramethyl-1,3,2-dioxaborolan-2-yl)-1,3,2-dioxaborolane (2.08 g, 8.20 mmol, 1.50 eq), KOAc (1.61 g, 16.4 mmol, 3.00 eq) and Pd(dppf)Cl₂ (400 mg, 546 μmol, 0.100 eq) in dioxane (5 mL) was degassed and purged with N₂ for 3 times at 25 °C, and then the mixture was stirred at 100 °C for 12 h under N₂ atmosphere. LC-MS showed the desired mass was detected. The reaction mixture was diluted with H₂O 40 mL and extracted with EtOAc (50 mL*3). The combined organic layers were concentrated under reduced pressure. The residue was purified by column chromatography (SiO₂, Petroleum ether:Ethyl acetate=1:0 to 0:1) to give the title compound (176-3) (1.20 g, 2.87 mmol, 52.4% yield, 72.4% purity) as a brown solid. LCMS (ES⁺): m/z 304.1 [M+H]⁺.

Synthesis of (4S)-1-[3-(2-chloro-5-fluoro-pyrimidin-4-yl)phenyl]-4-hydroxy-pyrrolidin-2-one (176-4). A mixture of (4S)-4-hydroxy-1-[3-(4,4,5,5-tetramethyl-1,3,2-dioxaborolan-2-yl)phenyl]pyrrolidin-2-one (176-3) (550 mg, 1.81 mmol, 1.00 eq), 2,4-dichloro-5-fluoro-pyrimidine (363 mg, 2.18 mmol, 1.20 eq), Na₂CO₃ (384 mg, 3.63 mmol, 2.00 eq), Pd(dppf)Cl₂ (132 mg, 181 μmol, 0.100 eq) in dioxane (5 mL) and H₂O (1 mL) was degassed and purged with N₂ for 3 times at 25 °C, and then the mixture was stirred at 100 °C for 12 h under N₂ atmosphere. LC-MS showed desired mass was detected. The reaction mixture was diluted with H₂O 20 mL and extracted with EtOAc (30 mL*3). The combined organic layers were concentrated under reduced pressure. The residue was purified by column chromatography (SiO₂, Petroleum ether:Ethyl acetate=1:0 to 0:1) to give the title compound (176-4) (600 mg, 1.37 mmol, 75.6% yield, 70.4% purity) as a yellow solid. LCMS (ES⁺): m/z 308.1 [M+H]⁺.

Synthesis of (4S)-1-[3-[2-[(1-acetyl-4-piperidyl)amino]-5-fluoro-pyrimidin-4-yl]phenyl]-4-hydroxy-pyrrolidin-2-one (Compound 230). The reaction was set up in two batches. A mixture of (4S)-1-[3-(2-chloro-5-fluoro-pyrimidin-4-yl)phenyl]-4-hydroxy-pyrrolidin-2-one (176-4) (290 mg, 942 μmol, 1.00 eq), 1-(4-amino-1-piperidyl)ethanone (201 mg, 1.41 mmol, 1.50 eq), Cs₂CO₃ (921 mg, 2.83 mmol, 3.00 eq), BINAP (117 mg, 188 μmol, 0.200 eq) and Pd₂(dba)₃ (172 mg, 188 μmol, 0.200 eq) in dioxane (5 mL) was degassed and purged with N₂ for 3 times at 25 °C, and then the mixture was stirred at 100 °C for 12 h under N₂ atmosphere. LC-MS showed the desired mass was detected. The reaction mixture was diluted with H₂O 20 mL and extracted with EtOAc (20 mL*3). The combined organic layers were concentrated under reduced pressure. The residue was purified by prep-HPLC (column: Phenomenex Luna C18 75*30 mm*3 um; mobile phase: [H₂O (0.2% FA)-ACN]; gradient: 15%-45% B over 8.0 min) to give the title compound (Compound 230) (5.00 mg, 11.6 μmol, 0.62% yield, 96.6% purity) as a yellow solid. LCMS (ES⁺): m/z 414.2 [M+H]⁺.

### Example 177. Synthesis of 1-(4-((4-(4-cyclopropyl-1H-pyrazol-1-yl)-5-fluoropyrimidin-2-yl)amino)piperidin-1-yl)ethan-1-one (Compound 219)

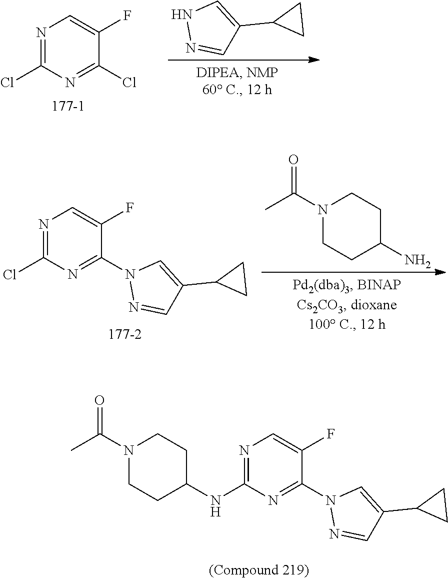

Synthesis of 2-chloro-4-(4-cyclopropyl-1H-pyrazol-1-yl)-5-fluoropyrimidine (177-2). To a solution of 2,4-dichloro-5-fluoropyrimidine (177-1) (300 mg, 1.80 mmol, 1.00 eq) and 4-cyclopropyl-1H-pyrazole (233 mg, 2.15 mmol, 1.20 eq) in NMP (2 mL) was added DIPEA (464 mg, 3.59 mmol, 626 μL, 2.00 eq) at 15 °C. The mixture was stirred at 60 °C for 12 h. LC-MS showed that 2,4-dichloro-5-fluoropyrimidine (177-1) was consumed completely and one main peak with desired mass was detected. The mixture was added into water (10 mL), then extracted with EtOAc (5 mL*3), the combined organic layers were washed with brine (15 mL) dried over Na₂SO₄, filtered and concentrated under reduced pressure. The residue was purified by prep-TLC (SiO₂, PE/EtOAc=3/1) to give title Compound (177-2) (180 mg, 754 μmol, 42.0% yield) as a yellow solid. LCMS (ES⁺): m/z 239.1 [M+H]⁺.

Synthesis of 1-(4-((4-(4-cyclopropyl-1H-pyrazol-1-yl)-5-fluoropyrimidin-2-yl)amino)piperidin-1-yl)ethan-1-one (Compound 219). To a solution of 2-chloro-4-(4-cyclopropyl-1H-pyrazol-1-yl)-5-fluoropyrimidine (177-2) (160 mg, 670 μmol, 1.00 eq) and 1-(4-aminopiperidin-1-yl)ethan-1-one (95.3 mg, 670 μmol, 1.00 eq) in dioxane (4 mL) were added Cs₂CO₃ (437 mg, 1.34 mmol, 2.00 eq), BINAP (41.8 mg, 67.0 μmol, 0.100 eq) and Pd₂(dba)₃ (61.4 mg, 67.0 μmol, 0.100 eq) at 15 °C. The mixture was stirred at 100 °C for 12 h under N₂. LC-MS showed that 2-chloro-4-(4-cyclopropyl-1H-pyrazol-1-yl)-5-fluoropyrimidine (177-2) was consumed completely and one main peak with desired mass was detected. The mixture was filtered and the filtrate was concentrated under reduced press The residue was purified by prep-HPLC (column: Waters Xbridge BEH C18 100*25 mm*10 um; mobile phase: [H₂O (10 mM NH₄HCO₃)-ACN]; gradient: 20%-50% B over 8.0 min) to give title compound (Compound 219) (14.7 mg, 41.7 μmol, 6.21% yield, 97.6% purity) as a white solid. LCMS (ES⁺): m/z 345.2 [M+H]⁺.

### Example 178. Synthesis of rac of trans-N-((1r,4r)-4-((5-fluoro-4-(4-(2-oxopyridin-1 (2H)-yl)-1H-pyrazol-1-yl)pyrimidin-2-yl)amino)cyclohexyl)acetamide (Compound 260)

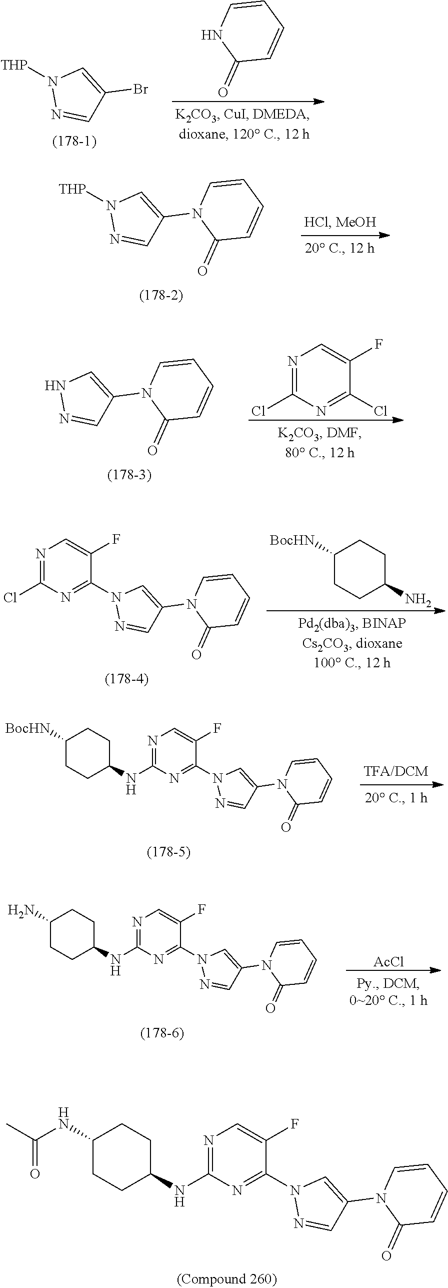

Synthesis of 1-(1-(tetrahydro-2H-pyran-2-yl)-1H-pyrazol-4-yl)pyridin-2(1H)-one (178-2). To a solution of 4-bromo-1-(tetrahydro-2H-pyran-2-yl)-1H-pyrazole (178-1) (18.0 g, 77.9 mmol, 1.00 eq) in dioxane (150 mL) were added pyridin-2(1H)-one (7.41 g, 77.9 mmol, 1.00 eq), CuI (1.48 g, 7.79 mmol, 0.100 eq), DMEDA (11.7 g, 133 mmol, 14.3 mL, 1.70 eq) and K₂CO₃ (32.3 g, 234 mmol, 3.00 eq) at 20 °C. The mixture was stirred at 120 °C for 12 h under N₂. LC-MS showed that 4-bromo-1-(tetrahydro-2H-pyran-2-yl)-1H-pyrazole (178-1) was not consumed completely and one peak with the desired mass was detected. The mixture was filtered and the filtrate was concentrated under reduced pressure. The residue was purified by column chromatography (SiO₂, Petroleum ether:Ethyl acetate=1:0 to 0:1) to give the title compound (178-2) (13.0 g, 53.0 mmol, 68.0% yield, n/a purity) as a white solid. LCMS (ES⁺): m/z 246.1 [M+H]⁺.

Synthesis of 1-(1H-pyrazol-4-yl)pyridin-2(1H)-one (178-3). To a solution of 1-(1-(tetrahydro-2H-pyran-2-yl)-1H-pyrazol-4-yl)pyridin-2(1H)-one (178-2) (13.0 g, 53.0 mmol, 1.00 eq) in MeOH (140 mL) was added HCl (12 M, 7 mL, 1.58 eq) at 20 °C. The mixture was stirred at 20 °C for 12 h. LC-MS showed that 1-(1-(tetrahydro-2H-pyran-2-yl)-1H-pyrazol-4-yl)pyridin-2(1H)-one (178-2) was not consumed completely and one main peak with the desired mass was detected. The mixture was concentrated under reduced pressure to give the title compound (178-3) (10.0 g, crude, HCl) as a white solid. LCMS (ES⁺): m/z 162.1 [M+H]⁺.

Synthesis of 1-(1-(2-chloro-5-fluoropyrimidin-4-yl)-1H-pyrazol-4-yl)pyridin-2 (1H)-one (178-4). To a solution of 1-(1H-pyrazol-4-yl)pyridin-2(1H)-one (178-3) (5.00 g, 25.3 mmol, 1.00 eq, HCl) in DMF (50 mL) were added 2,4-dichloro-5-fluoropyrimidine (16.9 g, 101 mmol, 4.00 eq) and K₂CO₃ (10.5 g, 76.0 mmol, 3.00 eq) at 20 °C. The mixture was stirred at 80 °C for 12 h. LC-MS showed that 1-(1H-pyrazol-4-yl)pyridin-2(1H)-one (178-3) was consumed completely and one main peak with the desired mass was detected. The mixture was filtered and the filter cake was dried in a vacuum. The crude product was triturated with PE (30 ml), filtered and the filter cake was dried in a vacuum to give the title compound (178-4) (3.00 g, 10.3 mmol, 40.7% yield, n/a purity) as a light yellow solid. LCMS (ES⁺): m/z 292.1 [M+H]⁺.

Synthesis of trans-tert-butyl ((1r,4r)-4-((5-fluoro-4-(4-(2-oxopyridin-1 (2H)-yl)-1H-pyrazol-1-yl)pyrimidin-2-yl)amino)cyclohexyl)carbamate (178-5). To a solution of 1-(1-(2-chloro-5-fluoropyrimidin-4-yl)-1H-pyrazol-4-yl)pyridin-2(1H)-one (178-4) (1.00 g, 3.43 mmol, 1.00 eq) in dioxane (15 mL) were added trans-tert-butyl ((1r,4r)-4-aminocyclohexyl)carbamate (808 mg, 3.77 mmol, 1.10 eq), Cs₂CO₃ (2.23 g, 6.86 mmol, 2.00 eq), BINAP (213 mg, 343 μmol, 0.100 eq) and Pd₂(dba)₃ (314 mg, 343 μmol, 0.100 eq) at 20 °C. The mixture was stirred at 100 °C for 12 h under N₂. LC-MS showed that 1-(1-(2-chloro-5-fluoropyrimidin-4-yl)-1H-pyrazol-4-yl)pyridin-2(1H)-one (178-4) was consumed completely and one main peak with the desired mass was detected. The mixture was filtered and the filtrate was concentrated under reduced pressure. The crude product was triturated with PE (20 mL), filtered and the filter cake was dried in a vacuum to give the title compound (178-5) (1.60 g, 3.41 mmol, 99.4% yield, n/a purity) as a yellow solid. LCMS (ES⁺): m/z 470.2 [M+H]⁺.

Synthesis of trans-1-(1-(2-(((1r,4r)-4-aminocyclohexyl)amino)-5-fluoropyrimidin-4-yl)-1H-pyrazol-4-yl)pyridin-2(1H)-one (178-6). To a solution of trans-tert-butyl ((1r,4r)-4-((5-fluoro-4-(4-(2-oxopyridin-1 (2H)-yl)-1H-pyrazol-1-yl)pyrimidin-2-yl)amino)cyclohexyl)carbamate (178-5) (1.00 g, 2.13 mmol, 1.00 eq) in DCM (10 mL) was added TFA (4.61 g, 40.4 mmol, 3 mL, 19.0 eq) at 20 °C. The mixture was stirred at 20 °C for 1 h. LC-MS showed trans-tert-butyl ((1r,4r)-4-((5-fluoro-4-(4-(2-oxopyridin-1 (2H)-yl)-1H-pyrazol-1-yl)pyrimidin-2-yl)amino)cyclohexyl)carbamate (178-5) was consumed completely and one main peak with the desired mass was detected. The crude product the title compound (178-6) (1.00 g, crude, TFA) in DCM (10 mL) as a yellow liquid was used into the next step without further purification. LCMS (ES⁺): m/z 370.2 [M+H]⁺.

Synthesis of trans-N-((1r,4r)-4-((5-fluoro-4-(4-(2-oxopyridin-1 (2H)-yl)-1H-pyrazol-1-yl)pyrimidin-2-yl)amino)cyclohexyl)acetamide (Compound 260). To a solution of trans-1-(1-(2-(((1r,4r)-4-aminocyclohexyl)amino)-5-fluoropyrimidin-4-yl)-1H-pyrazol-4-yl)pyridin-2(1H)-one (178-6) (1.00 g, 2.07 mmol, 1.00 eq, TFA) in DCM (10 mL) was added Py (11.2 g, 142 mmol, 11.5 mL, 68.7 eq), acetyl chloride (163 mg, 2.08 mmol, 148 μL, 1.00 eq) at 0 °C. The mixture was stirred at 20 °C for 1 h. LC-MS showed trans-1-(1-(2-(((1r,4r)-4-aminocyclohexyl)amino)-5-fluoropyrimidin-4-yl)-1H-pyrazol-4-yl)pyridin-2(1H)-one (178-6) was not consumed completely and one main peak with the desired mass was detected. The mixture was stirred at 20 °C for 1 h. LC-MS showed trans-1-(1-(2-(((1r,4r)-4-aminocyclohexyl)amino)-5-fluoropyrimidin-4-yl)-1H-pyrazol-4-yl)pyridin-2(1H)-one (178-6) was consumed completely and one main peak with the desired mass was detected. The mixture was concentrated under reduced pressure. The residue was purified by prep-HPLC (column: Waters Xbridge BEH C18 100*30 mm*10 um; mobile phase: [H₂O (10 mM NH₄HCO₃)-ACN]; gradient: 15%-45% B over 8.0 min) to give the title compound (Compound 260) (92.0 mg, 207 μmol, 10.0% yield, 92.6% purity) as a white solid. LCMS (ES⁺): m/z 412.1 [M+H]⁺.

### Example 179. Synthesis of 1-(4-((4-(3-(1H-imidazol-2-yl)phenyl)-5-fluoropyrimidin-2-yl)amino)piperidin-1-yl)ethan-1-one (Compound 239)

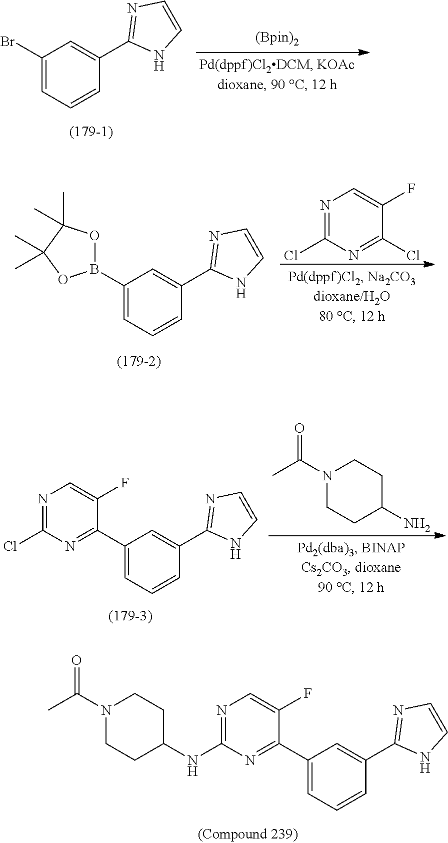

Synthesis of 2-(3-(4,4,5,5-tetramethyl-1,3,2-dioxaborolan-2-yl)phenyl)-1H-imidazole (179-2). To a solution of 2-(3-bromophenyl)-1H-imidazole (179-1) (2.00 g, 8.97 mmol, 1.00 eq) and 4,4,5,5-tetramethyl-2-(4,4,5,5-tetramethyl-1,3,2-dioxaborolan-2-yl)-1,3,2-dioxaborolane (2.73 g, 10.8 mmol, 1.20 eq) in dioxane (40 mL) was added KOAc (2.20 g, 22.4 mmol, 2.50 eq) and Pd(dppf)Cl₂·CH₂Cl₂ (366 mg, 448 μmol, 0.0500 eq) at 15 °C. The mixture was stirred at 90 °C for 12 h under N₂. LCMS showed that 2-(3-bromophenyl)-1H-imidazole (179-1) was consumed completely and one main peak with desired mass was detected. The mixture was concentrated under reduced pressure. The residue was purified by reverse-MPLC (FA condition) to give title compound (179-2) (2.0 g, crude) as a brown oil. LCMS (ES⁺): m/z 271.0 [M+H]⁺.

Synthesis of 4-(3-(1H-imidazol-2-yl)phenyl)-2-chloro-5-fluoropyrimidine (179-3). To a solution of (4,4,5,5-tetramethyl-1,3,2-dioxaborolan-2-yl)phenyl)-1H-imidazole (179-2) (2.00 g, 10.6 mmol, 1.00 eq) and 2,4-dichloro-5-fluoro-pyrimidine (1.78 g, 10.6 mmol, 1.00 eq) in dioxane (30 mL) and H₂O (3 mL) were added Na₂CO₃ (2.26 g, 21.3 mmol, 2.00 eq) and Pd(dppf)Cl₂ (389 mg, 531 μmol, 4.99e-2 eq) at 15 °C. The mixture was stirred at 80 °C for 12 h under N₂. LCMS showed that (4,4,5,5-tetramethyl-1,3,2-dioxaborolan-2-yl)phenyl)-1H-imidazole (179-2) was consumed completely and one main peak with desired mass was detected. The mixture was filtered and the filtrate was concentrated under reduced pressure. The residue was purified by prep-TLC (SiO₂, PE/EtOAc=0/1) to give title compound (179-3) (1.70 g, 6.19 mmol, 58.2% yield, n/a purity) as a brown solid. LCMS (ES⁺): m/z 275.0 [M+H]⁺.

Synthesis of 1-(4-((4-(3-(1H-imidazol-2-yl)phenyl)-5-fluoropyrimidin-2-yl)amino)piperidin-1-yl)ethan-1-one (Compound 239). To a solution of 4-(3-(1H-imidazol-2-yl)phenyl)-2-chloro-5-fluoropyrimidine (179-3) (500 mg, 1.82 mmol, 1.00 eq) and 1-(4-amino-1-piperidyl)ethanone (259 mg, 1.82 mmol, 1.00 eq) in dioxane (6 mL) were added Cs₂CO₃ (1.19 g, 3.64 mmol, 2.00 eq), BINAP (113 mg, 181 μmol, 9.97e-2 eq) and Pd₂(dba)₃ (167 mg, 182 μmol, 0.100 eq) at 15 °C. The mixture was stirred at 90 °C for 12 h under N₂. LCMS showed that 4-(3-(1H-imidazol-2-yl)phenyl)-2-chloro-5-fluoropyrimidine (179-3) remained and one peak with desired mass was detected. The mixture was filtered and the filtrate was concentrated under reduced pressure. The residue was purified by prep-HPLC (column: Waters Xbridge BEH C18 250*50 mm*10 um; mobile phase: [H₂O (10 mM NH₄HCO₃)-ACN]; gradient: 20%-50% B over 10.0 min) to give title compound (Compound 239) (32.0 mg, 81.2 μmol, 96.6% yield, 96.6% purity) as a white solid. LCMS (ES⁺): m/z 381.2 [M+H]⁺.

### Example 180. Synthesis of (R)-1-(4-((5-fluoro-4-(3-(3-hydroxypiperidin-1-yl)phenyl)pyrimidin-2-yl)amino)piperidin-1-yl)ethan-1-one (Compound 209)

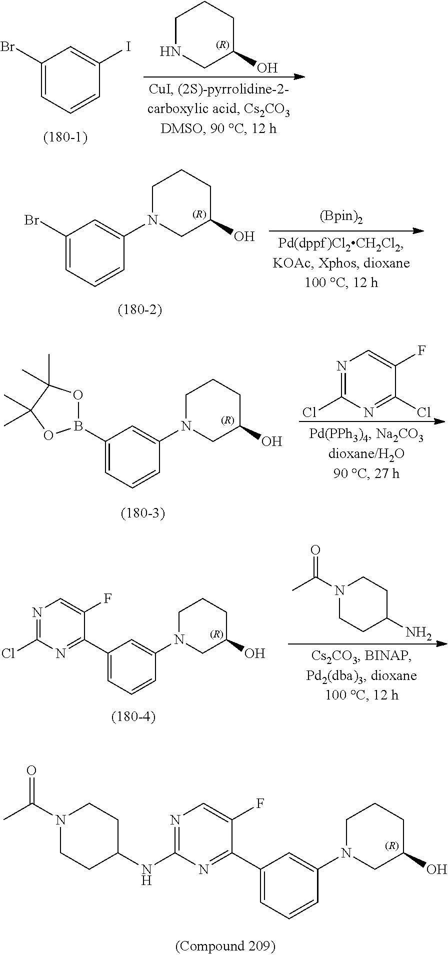

Synthesis of (R)-1-(3-bromophenyl)piperidin-3-ol (180-2). To a solution of 1-bromo-3-iodobenzene (180-1) (5.09 g, 18.0 mmol, 2.29 mL, 1.00 eq) in DMSO (50 mL) were added (R)-piperidin-3-ol (2.00 g, 19.8 mmol, 1.10 eq), Cs₂CO₃ (17.6 g, 53.9 mmol, 3.00 eq), (2S)-pyrrolidine-2-carboxylic acid (414 mg, 3.60 mmol, 0.200 eq) and CuI (342 mg, 1.80 mmol, 0.100 eq) at 25 °C under N₂ atmosphere. The mixture was stirred at 90 °C for 12 h under N₂. TLC (SiO₂, Petroleum ether:Ethyl acetate=1:1) showed 1-bromo-3-iodobenzene (180-1) was remaining, and two new spots formed. LC-MS showed the desired mass was detected. Water (50 mL) was added to the mixture at 25 °C. The reaction mixture was extracted with EtOAC (30 mL*5). The combined organic layers were dried over anhydrous sodium sulfate, filtered and the filtrate was concentrated under reduced to give a residue. The residue was purified by column chromatography (SiO₂, Petroleum ether:Ethyl acetate=3:1 to 1:1) to give the title compound (180-2) (1.57 g, 6.13 mmol, 34.1% yield) as a pink oil. LCMS (ES⁺): m/z 256.1 [M+H]⁺.

Synthesis of (R)-1-(3-(4,4,5,5-tetramethyl-1,3,2-dioxaborolan-2-yl)phenyl)piperidin-3-ol (180-3). To a solution of (R)-1-(3-bromophenyl)piperidin-3-ol (180-2) (1.50 g, 5.86 mmol, 1.00 eq) in dioxane (35 mL) were added 4,4,5,5-tetramethyl-2-(4,4,5,5-tetramethyl-1,3,2-dioxaborolan-2-yl)-1,3,2-dioxaborolane (1.93 g, 7.61 mmol, 1.30 eq), KOAc (1.72 g, 17.6 mmol, 3.00 eq), XPhos (156 mg, 328 μmol, 0.0560 eq) and Pd(dppf)Cl₂·CH₂Cl₂ (478 mg, 586 μmol, 0.100 eq) at 25 °C under N₂ atmosphere. The mixture was stirred at 100 °C for 12 h under N₂. LC-MS showed (R)-1-(3-bromophenyl)piperidin-3-ol (180-2) was consumed completely and the desired mass was detected. Water (30 mL) was added to the mixture at 25 °C. The reaction mixture was extracted with EtOAC (20 mL*5). The organic layers were dried over anhydrous sodium sulfate, filtered and the filtrate was concentrated under reduced to give a residue. The residue was purified by column chromatography (SiO₂, Petroleum ether:Ethyl acetate=4:1 to 1:1) to give the title compound (180-3) (2.12 g, crude) as a yellow oil. LCMS (ES⁺): m/z 304.2 [M+H]⁺.

Synthesis of (R)-1-(3-(2-chloro-5-fluoropyrimidin-4-yl)phenyl)piperidin-3-ol (180-4). Four batches in parallel were setup. To a solution of (R)-1-(3-(4,4,5,5-tetramethyl-1,3,2-dioxaborolan-2-yl)phenyl)piperidin-3-ol (180-3) (250 mg, 825 μmol, 1.00 eq) in dioxane (4 mL) and H₂O (0.4 mL) were added 2,4-dichloro-5-fluoropyrimidine (151 mg, 907 mol, 1.10 eq), Na₂CO₃ (175 mg, 1.65 mmol, 240 μL, 2.00 eq) and Pd(PPh₃)₄ (19.1 mg, 16.5 μmol, 0.0200 eq) at 25 °C under N₂ atmosphere. The mixture was stirred at 90 °C for 12 h under N₂. One batch in parallel was monitored in random. LC-MS showed ~16.7% of (R)-1-(3-(4,4,5,5-tetramethyl-1,3,2-dioxaborolan-2-yl)phenyl)piperidin-3-ol (180-3) was remaining, and ~22.4% of the desired mass was detected. Combined these four batches. Water (15 mL) was added to the mixture at 25 °C. The reaction mixture was extracted with EtOAC (10 mL*5). The organic layers were dried over anhydrous sodium sulfate, filtered and the filtrate was concentrated under reduced to give a residue. The residue was purified by column chromatography (SiO₂, Petroleum ether:Ethyl acetate=3:1 to 2:1) to give the title compound (180-4) (345 mg, 1.12 mmol, 34.0% yield) as a yellow solid. LCMS (ES⁺): m/z 308.2 [M+H]⁺.

Synthesis of (R)-1-(4-((5-fluoro-4-(3-(3-hydroxypiperidin-1-yl)phenyl)pyrimidin-2-yl)amino)piperidin-1-yl)ethan-1-one (Compound 209). To a solution of (R)-1-(3-(2-chloro-5-fluoropyrimidin-4-yl)phenyl)piperidin-3-ol (180-4) (345 mg, 1.12 mmol, 1.00 eq) in dioxane (6 mL) were added 1-(4-aminopiperidin-1-yl)ethan-1-one (159 mg, 1.12 mmol, 1.00 eq), Cs₂CO₃ (1.10 g, 3.36 mmol, 3.00 eq), BINAP (69.8 mg, 112 μmol, 0.100 eq) and Pd₂(dba)₃ (103 mg, 112 μmol, 0.100 eq) at 25 °C under N₂ atmosphere. The mixture was stirred at 100 °C for 12 h under N₂. LC-MS showed (R)-1-(3-(2-chloro-5-fluoropyrimidin-4-yl)phenyl)piperidin-3-ol (180-4) was consumed completely and the desired mass was detected. The reaction mixture was filtered with THF (40 mL) and the filtrate was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 26%-56% B over 8.0 min) to give the title compound (Compound 209) (95.0 mg, 229 μmol, 20.4% yield, 99.7% purity) as a light yellow solid. LCMS (ES⁺): m/z 414.1 [M+H]⁺.

### Example 181. Synthesis of (S)-1-(4-((5-fluoro-4-(3-(3-hydroxypiperidin-1-yl)phenyl)pyrimidin-2-yl)amino)piperidin-1-yl)ethan-1-one (Compound 210)

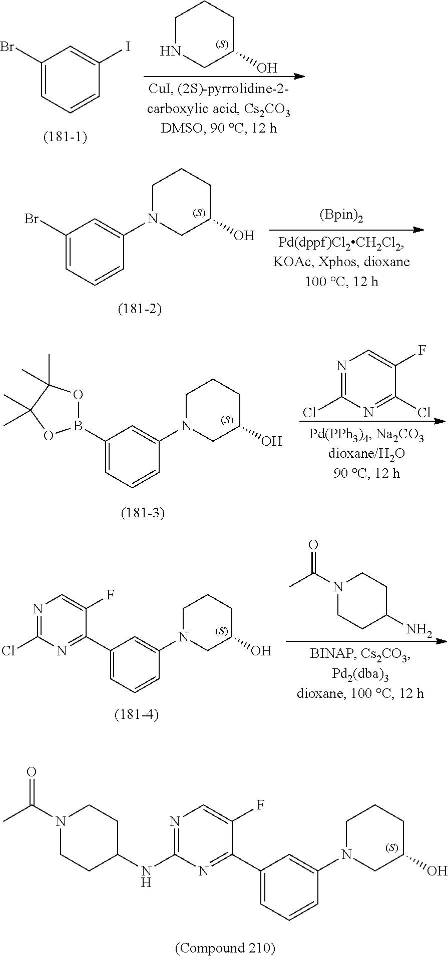

Synthesis of (S)-1-(3-bromophenyl)piperidin-3-ol (181-2). To a solution of 1-bromo-3-iodobenzene (181-1) (5.00 g, 17.7 mmol, 2.25 mL, 1.00 eq) and (S)-piperidin-3-ol (1.97 g, 19.4 mmol, 1.10 eq) in DMSO (50 mL) were added Cs₂CO₃ (17.3 g, 53.0 mmol, 3.00 eq), (2S)-pyrrolidine-2-carboxylic acid (407 mg, 3.53 mmol, 0.200 eq) and CuI (337 mg, 1.77 mmol, 0.100 eq) at 20 °C under N₂ atmosphere. The mixture was stirred at 90 °C for 12 h under N₂. TLC (SiO₂, Petroleum ether:Ethyl acetate=1:1) showed 1-bromo-3-iodobenzene (181-1) was remaining, and one new spot formed. LCMS the desired mass was detected. Water (50 mL) was added to the mixture at 25 °C. Then the reaction mixture was extracted with EtOAc (40 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=3:1 to 1:1) to give the title compound (181-2) (1.60 g, 6.25 mmol, 35.3% yield) as a colorless oil. LCMS (ES⁺): m/z 256.0 [M+H]⁺.

Synthesis of (S)-1-(3-(4,4,5,5-tetramethyl-1,3,2-dioxaborolan-2-yl)phenyl)piperidin-3-ol (181-3). To a solution of (S)-1-(3-bromophenyl)piperidin-3-ol (181-2) (1.60 g, 6.25 mmol, 1.00 eq) in dioxane (40 mL) were added 4,4,5,5-tetramethyl-2-(4,4,5,5-tetramethyl-1,3,2-dioxaborolan-2-yl)-1,3,2-dioxaborolane (2.06 g, 8.12 mmol, 1.30 eq), XPhos (167 mg, 3 50 μmol, 0.0560 eq), KOAc (1.84 g, 18.7 mmol, 3.00 eq) and Pd(dppf)Cl₂·CH₂Cl₂ (510 mg, 625 μmol, 0.100 eq) at 20 °C under N₂ atmosphere. The mixture was stirred at 100 °C for 12 h under N₂. LCMS showed (S)-1-(3-bromophenyl)piperidin-3-ol (181-2) was consumed completely and the desired mass was detected. Water (40 mL) was added to the mixture at 20 °C. Then the reaction mixture was extracted with EtOAc (20 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=3:1 to 1:1) to give the title compound (181-3) (1.80 g, 5.94 mmol, 95.0% yield) as a yellow oil. LCMS (ES⁺): m/z 304.2 [M+H]⁺.

Synthesis of (S)-1-(3-(2-chloro-5-fluoropyrimidin-4-yl)phenyl)piperidin-3-ol (181-4). To a solution of (S)-1-(3-(4,4,5,5-tetramethyl-1,3,2-dioxaborolan-2-yl)phenyl)piperidin-3-ol (181-3) (1.00 g, 3.30 mmol, 1.00 eq) and 2,4-dichloro-5-fluoro-pyrimidine (551 mg, 3.30 mmol, 1.00 eq) in dioxane (15 mL) and H₂O (1.5 mL) were added Na₂CO₃ (699 mg, 6.60 mmol, 2.00 eq) and Pd(PPh₃)₄ (76.2 mg, 66.0 μmol, 0.0200 eq) at 20 °C under N₂ atmosphere. The mixture was stirred at 90 °C for 12 h under N₂. LCMS showed (S)-1-(3-(4,4,5,5-tetramethyl-1,3,2-dioxaborolan-2-yl)phenyl)piperidin-3-ol (181-3) was consumed completely and the desired mass was detected. Water (15 mL) was added to the mixture at 25 °C. Then the reaction mixture was extracted with EtOAc (10 mL*3). The combined organic layers were dried over anhydrous sodium sulfate, filtered and the filtrate was concentrated under reduced pressure to give a residue. The residue was purified by column chromatography (SiO₂, Petroleum ether:Ethyl acetate=3:1 to 1:1) to give the title compound (181-4) (625 mg, 1.73 mmol, 52.6% yield, 85.4% purity) as a yellow solid. LCMS (ES⁺): m/z 308.0 [M+H]⁺.

Synthesis of (S)-1-(4-((5-fluoro-4-(3-(3-hydroxypiperidin-J-yl)phenyl)pyrimidin-2-yl)amino)piperidin-1-yl)ethan-1-one (Compound 210). To a solution of (S)-1-(3-(2-chloro-5-fluoropyrimidin-4-yl)phenyl)piperidin-3-ol (181-4) (300 mg, 975 μmol, 1.00 eq) and 1-(4-amino-1-piperidyl)ethanone (139 mg, 975 μmol, 1.00 eq) in dioxane (4 mL) was added Cs₂CO₃ (953 mg, 2.92 mmol, 3.00 eq) and BINAP (60.7 mg, 97.5 μmol, 0.100 eq) and Pd₂(dba)₃ (89.3 mg, 97.5 μmol, 0.100 eq) at 20 °C under N₂ atmosphere. The mixture was stirred at 100 °C for 12 h under N₂. LCMS showed (S)-1-(3-(2-chloro-5-fluoropyrimidin-4-yl)phenyl)piperidin-3-ol (181-4) was consumed completely and the desired mass was detected. The reaction mixture filtered and the filtrate was concentrated under reduced pressure to give a residue. The residue was purified by prep-HPLC (column: Phenomenex luna C18 100*40 mm*5 um; mobile phase: [H₂O (0.2% FA)-ACN]; gradient: 1%-50% B over 8.0 min) to give the title compound (Compound 210) (75.0 mg, 181 μmol, 18.6% yield, 100% purity) as a white solid. LCMS (ES⁺): m/z 414.2 [M+H]⁺.

### Example 182. Synthesis of 1-(4-((5-fluoro-4-(3-(4-hydroxypiperidin-1-yl)phenyl)pyrimidin-2-yl)amino)piperidin-1-yl)ethan-1-one (Compound 211)

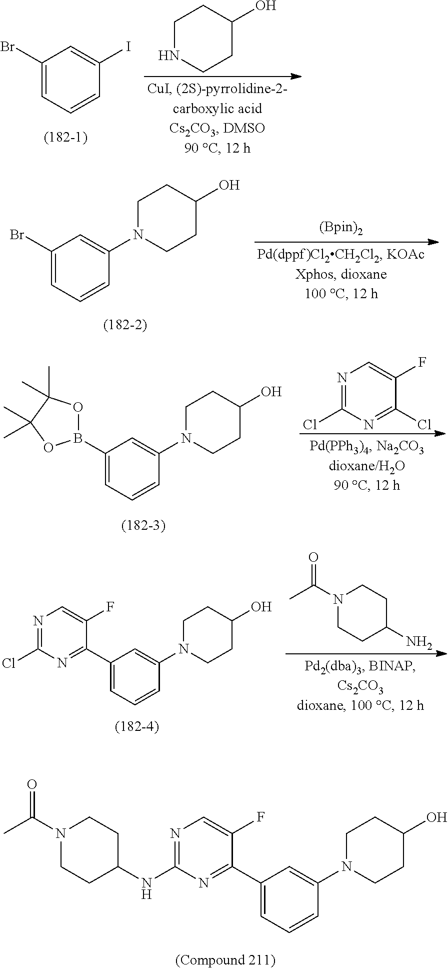

Synthesis of 1-(3-bromophenyl)piperidin-4-ol (182-2). To a solution of 1-bromo-3-iodobenzene (182-1) (4.20 g, 14.9 mmol, 1.89 mL, 1.00 eq) in DMSO (50 mL) were added piperidin-4-ol (1.65 g, 16.3 mmol, 1.10 eq), CuI (283 mg, 1.48 mmol, 0.100 eq), (2S)-pyrrolidine-2-carboxylic acid (342 mg, 2.97 mmol, 0.200 eq) and Cs₂CO₃ (14.5 g, 44.5 mmol, 3.00 eq) at 20 °C under N₂ atmosphere. The mixture was stirred at 90 °C for 12 h under N₂. LC-MS showed the desired mass was detected. Water (50 mL) was added to the mixture at 20 °C. Then the reaction mixture was extracted with EtOAc (30 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=3:1 to 1:1) to give the title compound (182-2) (2.10 g, 8.20 mmol, 55.2% yield) as a white solid. LCMS (ES⁺): m/z 256.1 [M+H]⁺.

Synthesis of 1-(3-(4,4,5,5-tetramethyl-1,3,2-dioxaborolan-2-yl)phenyl)piperidin-4-ol (182-3). To a solution of 1-(3-bromophenyl)piperidin-4-ol (182-2) (2.10 g, 8.20 mmol, 1.00 eq) in dioxane (40 mL) were added 4,4,5,5-tetramethyl-2-(4,4,5,5-tetramethyl-1,3,2-dioxaborolan-2-yl)-1,3,2-dioxaborolane (2.71 g, 10.7 mmol, 1.30 eq), XPhos (219 mg, 459 mol, 0.0560 eq), KOAc (2.41 g, 24.6 mmol, 3.00 eq) and Pd(dppf)Cl₂·CH₂Cl₂ (670 mg, 820 μmol, 0.100 eq) at 20 °C under N₂ atmosphere. The mixture was stirred at 100 °C for 12 h under N₂. LC-MS showed 1-(3-bromophenyl)piperidin-4-ol (182-2) was consumed completely and the desired mass was detected. Water (40 mL) was added to the mixture at 20 °C. Then the reaction mixture was extracted with EtOAc (20 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=1:1 to 1:2) to give the title compound (182-3) (2.00 g, 6.60 mmol, 80.5% yield) as a white solid. LCMS (ES⁺): m/z 304.2 [M+H]⁺.

Synthesis of 1-(3-(2-chloro-5-fluoropyrimidin-4-yl)phenyl)piperidin-4-ol (182-4). To a solution of 1-(3-(4,4,5,5-tetramethyl-1,3,2-dioxaborolan-2-yl)phenyl)piperidin-4-ol (182-3) (1.00 g, 3.30 mmol, 1.00 eq) in dioxane (20 mL) and H₂O (2 mL) were added 2,4-dichloro-5-fluoro-pyrimidine (661 mg, 3.96 mmol, 1.20 eq), Pd(PPh₃)₄ (76.2 mg, 66.0 μmol, 0.0200 eq) and Na₂CO₃ (699 mg, 6.60 mmol, 2.00 eq) at 20 °C under N₂ atmosphere. The mixture was stirred at 90 °C for 12 h under N₂. LC-MS showed 1-(3-(4,4,5,5-tetramethyl-1,3,2-dioxaborolan-2-yl)phenyl)piperidin-4-ol (182-3) was consumed completely and the desired mass was detected. Water (20 mL) was added to the mixture at 20 °C. Then the reaction mixture was extracted with EtOAc (10 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=1:1 to 1:2) to give the title compound (182-4) (850 mg, 2.76 mmol, 83.7% yield) as a white solid. LCMS (ES⁺): m/z 308.1 [M+H]⁺.

Synthesis of 1-(4-((5-fluoro-4-(3-(4-hydroxypiperidin-1-yl)phenyl)pyrimidin-2-yl)amino)piperidin-1-yl)ethan-1-one (Compound 211). To a solution of 1-(3-(2-chloro-5-fluoropyrimidin-4-yl)phenyl)piperidin-4-ol (182-4) (300 mg, 975 μmol, 1.00 eq) in dioxane (6 mL) were added 1-(4-amino-1-piperidyl)ethanone (139 mg, 975 μmol, 1.00 eq), BINAP (60.7 mg, 97.5 μmol, 0.100 eq), Pd₂(dba)₃ (89.3 mg, 97.5 μmol, 0.100 eq) and Cs₂CO₃ (794 mg, 2.44 mmol, 2.50 eq) at 20 °C under N₂ atmosphere. The mixture was stirred at 100 °C for 12 h under N₂. LC-MS showed 1-(3-(2-chloro-5-fluoropyrimidin-4-yl)phenyl)piperidin-4-ol (182-4) was consumed completely and the desired mass was detected. The reaction mixture was concentrated under reduced pressure to remove the solvent to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to give the title compound (Compound 211) (85.5 mg, 206 μmol, 21.2% yield, 99.7% purity) as a white solid. LCMS (ES⁺): m/z 414.2 [M+H]⁺.

### Example 183. Synthesis of trans-N-(4-((5-fluoro-4-(3-(4-hydroxypiperidin-1-yl)phenyl)pyrimidin-2-yl)amino)cyclohexyl)acetamide (Compound 250)

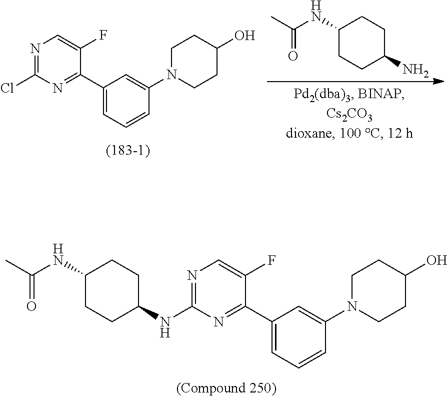

Synthesis of trans-N-(4-((5-fluoro-4-(3-(4-hydroxypiperidin-1-yl)phenyl)pyrimidin-2-yl)amino)cyclohexyl)acetamide (Compound 250). To a solution of 1-(3-(2-chloro-5-fluoropyrimidin-4-yl)phenyl)piperidin-4-ol (183-1) (370 mg, 1.20 mmol, 1.00 eq) and trans-N-(4-aminocyclohexyl) acetamide (376 mg, 2.40 mmol, 2.00 eq) in dioxane (1 mL) were added BINAP (74.9 mg, 120 μmol, 0.100 eq), Cs₂CO₃ (1.18 g, 3.61 mmol, 3.00 eq) and Pd₂(dba)₃ (110 mg, 120 μmol, 0.100 eq) at 25 °C under N₂ atmosphere. The mixture was stirred at 100 °C for 12 h under N₂. LCMS showed 1-(3-(2-chloro-5-fluoropyrimidin-4-yl)phenyl)piperidin-4-ol (183-1) was consumed completely and the desired mass was detected. The mixture was filtered and the filtrate was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-45% B over 8.0 min) to give the title compound (Compound 250) (105 mg, 241 μmol, 20.0% yield, 98.5% purity) as a white solid. LCMS (ES⁺): m/z 428.2 [M+H]⁺.

### Example 184. Synthesis of trans-4-((5-fluoro-4-(3-(4-hydroxypiperidin-1-yl)phenyl)pyrimidin-2-yl)amino)cyclohexane-1-carboxamide (Compound 251)

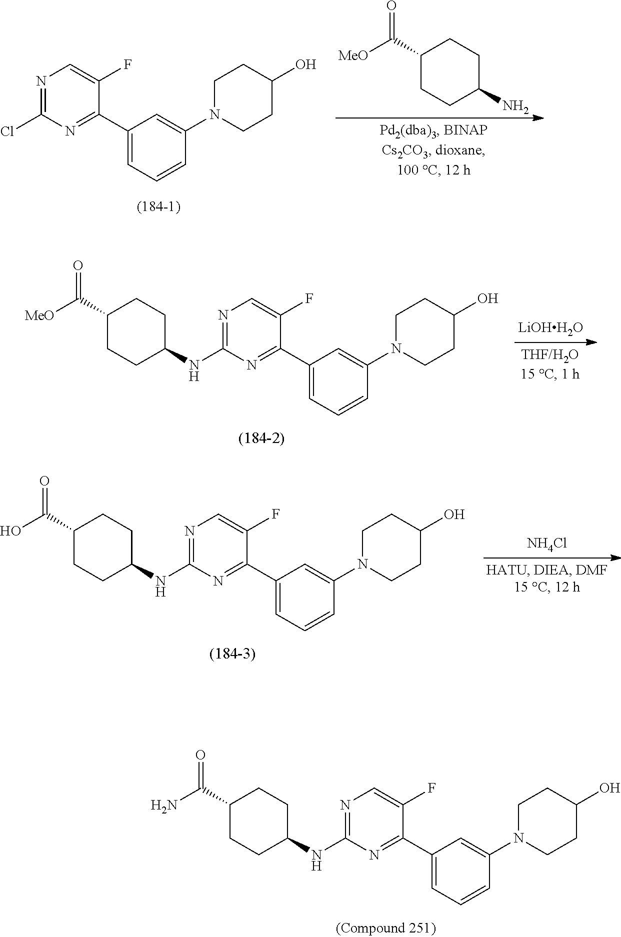

Synthesis of trans-methyl-4-((5-fluoro-4-(3-(4-hydroxypiperidin-1-yl)phenyl)pyrimidin-2-yl)amino)cyclohexane-1-carboxylate (184-2). To a stirred solution of 1-(3-(2-chloro-5-fluoropyrimidin-4-yl)phenyl)piperidin-4-ol (184-1) (700 mg, 2.27 mmol, 1.00 eq) and trans-methyl-4-aminocyclohexane-1-carboxylate (715 mg, 4.55 mmol, 2.00 eq) in dioxane (15 mL) were added Cs₂CO₃ (1.48 g, 4.55 mmol, 2.00 eq), BINAP (70.8 mg, 114 μmol, 0.0500 eq) and Pd₂(dba)₃ (104 mg, 114 μmol, 0.0500 eq) at 15 °C under N₂. The resulting mixture was stirred at 100 °C for 12 h under N₂. LCMS showed 1-(3-(2-chloro-5-fluoropyrimidin-4-yl)phenyl)piperidin-4-ol (184-1) was consumed mostly and the desired mass was detected. The mixture was diluted with H₂O (30 mL) and then extracted with EtOAc (20 mL*3). The combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (184-2) (600 mg, 1.40 mmol, 61.6% yield) as a yellow solid. LCMS (ES⁺): m/z 429.2 [M+H]⁺.

Synthesis of trans-4-((5-fluoro-4-(3-(4-hydroxypiperidin-1-yl)phenyl)pyrimidin-2-yl)amino)cyclohexane-1-carboxylic acid (184-3). To a stirred solution of trans-methyl-4-((5-fluoro-4-(3-(4-hydroxypiperidin-1-yl)phenyl)pyrimidin-2-yl)amino)cyclohexane-1-carboxylate (184-2) (600 mg, 1.40 mmol, 1.00 eq) in THE (6 mL) and H₂O (3 mL) was added LiOH·H₂O (118 mg, 2.80 mmol, 2.00 eq) at 15 °C. The resulting mixture was stirred at 15 °C for 1 h. LCMS showed trans-methyl-4-((5-fluoro-4-(3-(4-hydroxypiperidin-1-yl)phenyl)pyrimidin-2-yl)amino)cyclohexane-1-carboxylate (184-2) was consumed and the desired mass was detected. The mixture was concentrated under reduced pressure to remove THF, then the remaining aqueous phase was adjust pH to 2-3 by HCl (1M). The solid was precipitated, then the mixture was filtered and the filter cake was dried under reduced pressure to give the title compound (184-3) (400 mg, crude) as a yellow solid. LCMS (ES⁺): m/z 415.2 [M+H]⁺.

Synthesis of trans-4-((5-fluoro-4-(3-(4-hydroxypiperidin-1-yl)phenyl)pyrimidin-2-yl)amino)cyclohexane-1-carboxamide (Compound 251). To a stirred solution of trans-4-((5-fluoro-4-(3-(4-hydroxypiperidin-1-yl)phenyl)pyrimidin-2-yl)amino)cyclohexane-1-carboxylic acid (184-3) (400 mg, 965 μmol, 1.00 eq) and DIEA (249 mg, 1.93 mmol, 336 μL, 2.00 eq) in DMF (2 mL) were added HATU (550 mg, 1.45 mmol, 1.50 eq) and NH₄Cl (206 mg, 3.86 mmol, 4.00 eq) at 15 °C. The resulting mixture was stirred at 15 °C for 12 h. LCMS showed trans-4-((5-fluoro-4-(3-(4-hydroxypiperidin-1-yl)phenyl)pyrimidin-2-yl)amino)cyclohexane-1-carboxylic acid (184-3) was consumed completely and the desired mass was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 1%-35% B over 8.0 min) to give the title compound (Compound 251) (54.3 mg, 128 μmol, 13.3% yield, 97.7% purity) as a yellow solid. LCMS (ES⁺): m/z 414.2 [M+H]⁺.

### Example 185. Synthesis of (S)-1-(4-((5-fluoro-4-(3-(3-hydroxypyrrolidine-1-carbonyl)phenyl)pyrimidin-2-yl)amino)piperidin-1-yl)ethan-1-one (Compound 212)

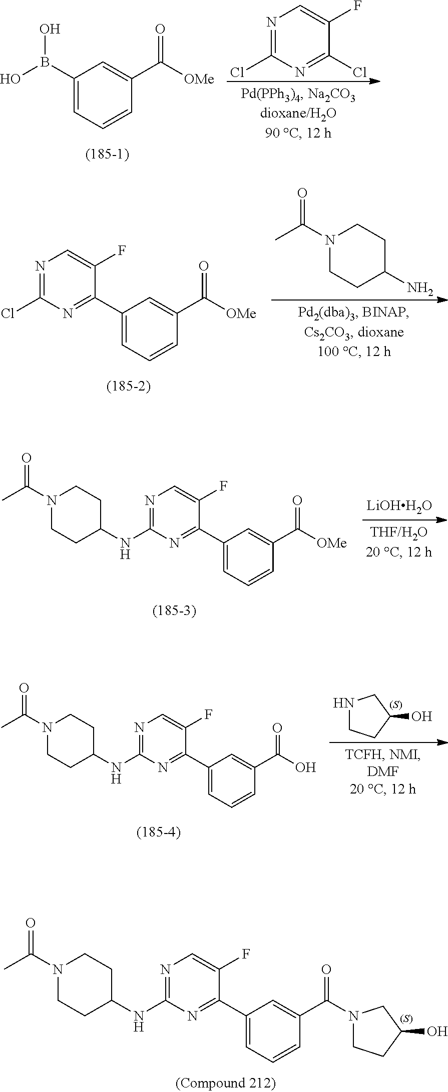

Synthesis of methyl 3-(2-chloro-5-fluoropyrimidin-4-yl)benzoate (185-2). To a solution of (3-(methoxycarbonyl)phenyl)boronic acid (185-1) (15.0 g, 83.4 mmol, 1.00 eq) and 2,4-dichloro-5-fluoropyrimidine (13.9 g, 83.4 mmol, 1.00 eq) in dioxane (300 mL) and H₂O (60 mL) were added Na₂CO₃ (17.7 g, 167 mmol, 2.00 eq) and Pd(PPh₃)₄ (9.63 g, 8.33 mmol, 0.100 eq) at 20 °C under N₂ atmosphere, the mixture was stirred at 80 °C for 12 h under N₂. LCMS showed the desired mass was detected. Water (100 mL) was added to the mixture, the mixture was extracted with EtOAc (80 mL*3), then the combined organic layers were dried over Na₂SO₄, filtered and the filtrate was concentrated under reduced pressure to give a residue. The residue was purified by column chromatography (SiO₂, Petroleum ether:Ethyl acetate=1:0 to 20:1) to give the title compound (185-2) (11.0 g, 41.3 mmol, 49.5% yield) as a white solid. LCMS (ES⁺): m/z 266.9 [M+H]⁺.

Synthesis of methyl 3-(2-((1-acetylpiperidin-4-yl)amino)-5-fluoropyrimidin-4-yl)benzoate (185-3). To a solution of methyl 3-(2-chloro-5-fluoropyrimidin-4-yl)benzoate (185-2) (3.90 g, 14.6 mmol, 1.00 eq) in dioxane (60 mL) were added BINAP (546 mg, 877 μmol, 0.0600 eq), Cs₂CO₃ (9.52 g, 29.2 mmol, 2.00 eq), Pd₂(dba)₃ (402 mg, 438 μmol, 0.0300 eq) and 1-(4-amino-1-piperidyl)ethanone (2.08 g, 14.6 mmol, 1.00 eq) at 20 °C under N₂ atmosphere. The mixture was stirred at 100 °C for 12 h under N₂. LCMS showed the desired mass was detected. Water (60 mL) was added to the mixture at 20 °C. Then the reaction mixture was extracted with EtOAc (50 mL*3). The organic layers were dried over anhydrous sodium sulfate, filtered and the filtrate was concentrated under reduced pressure to give a residue. The residue was purified by column chromatography (SiO₂, Dichloromethane:Methanol=20:1 to 10:1) to give the title compound (185-3) (4.77 g, 12.8 mmol, 87.6% yield) as a yellow oil. LCMS (ES⁺): m/z 373.0 [M+H]⁺.

Synthesis of 3-(2-((1-acetylpiperidin-4-yl)amino)-5-fluoropyrimidin-4-yl)benzoic acid (185-4). To a solution of methyl 3-(2-((1-acetylpiperidin-4-yl)amino)-5-fluoropyrimidin-4-yl)benzoate (185-3) (4.77 g, 12.8 mmol, 1.00 eq) in H₂O (20 mL) and THE (40 mL) was added LiOH·H₂O (2.15 g, 51.2 mmol, 4.00 eq) at 20 °C. The mixture was stirred at 20 °C for 12 h. LCMS showed methyl 3-(2-((1-acetylpiperidin-4-yl)amino)-5-fluoropyrimidin-4-yl)benzoate (185-3) was consumed completely and the desired mass was detected. The reaction was concentrated under reduced pressure to remove THF. Then HCl (1 M) was added dropwise until the pH had reached 3. The resulting precipitate was collected by filtration. The filter cake was dried in vacuo to give the title compound (185-4) (3.40 g, crude) as a white solid. LCMS (ES⁺): m/z 359.1 [M+H]⁺.

Synthesis of (S)-1-(4-((5-fluoro-4-(3-(3-hydroxypyrrolidine-1-carbonyl)phenyl)pyrimidin-2-yl)amino)piperidin-1-yl)ethan-1-one (Compound 212). To a solution of 3-(2-((1-acetylpiperidin-4-yl)amino)-5-fluoropyrimidin-4-yl)benzoic acid (185-4) (300 mg, 837 μmol, 1.00 eq) in DMF (3 mL) were added TCFH (517 mg, 1.84 mmol, 2.20 eq), NMI (412 mg, 5.02 mmol, 400 μL, 6.00 eq) and (3S)-pyrrolidin-3-ol (72.9 mg, 837 μmol, 67.7 μL, 1.00 eq) at 20 °C. The mixture was stirred at 20 °C for 12 h. LCMS showed 3-(2-((1-acetylpiperidin-4-yl)amino)-5-fluoropyrimidin-4-yl)benzoic acid (185-4) was consumed completely and the desired mass was detected. The organic layers were filtered and the filtrate was concentrated under reduced pressure to give a residue. The residue was purified by prep-HPLC (column: Phenomenex luna C18 100*40 mm*3 um; mobile phase: [H₂O (0.2% FA)-ACN]; gradient: 10%-40% B over 8.0 min) to give the title compound (Compound 212) (170 mg, 397 μmol, 47.5% yield, 99.9% purity) as a white solid. LCMS (ES⁺): m/z 428.2 [M+H]⁺.

### Example 186. Synthesis of trans-N-[4-[[5-fluoro-4-[3-[(3S)-3-hydroxypyrrolidine-1-carbonyl]phenyl]pyrimidin-2-yl]amino]cyclohexyl]acetamide (Compound 252)

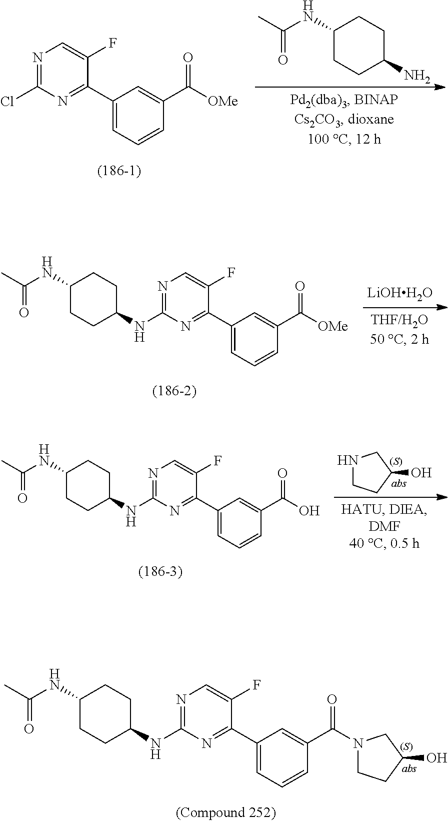

Synthesis of trans-methyl 3-[2-[(4-acetamidocyclohexyl)amino]-5-fluoro-pyrimidin-4-yl]benzoate (186-2). To a solution of methyl 3-(2-chloro-5-fluoro-pyrimidin-4-yl)benzoate (186-1) (1.71 g, 6.40 mmol, 1.00 eq) and trans-N-(4-aminocyclohexyl)acetamide (2.00 g, 12.8 mmol, 2.00 eq) in dioxane (20 mL) was added Cs₂CO₃ (4.17 g, 12.8 mmol, 2.00 eq) and Pd₂(dba)₃ (586 mg, 640 μmol, 0.100 eq) and BINAP (399 mg, 640 μmol, 0.100 eq) at 25 °C under N₂, the mixture was stirred at 100 °C for 12 h under N₂. LC-MS showed that the desired mass was detected. To the mixture was added H₂O (30 mL) and the mixture was extracted with EtOAc (30 mL*3). The combined organic layers were washed with brine (10 mL*3), dried over Na₂SO₄, filtered and the filtrate was concentrated under reduced pressure to give the crude product. The mixture was purified by MPLC (SiO₂, Ethyl acetate/Methanol=40/1 to 20/1) to give the title compound (186-2) (700 mg, 1.81 mmol, 28.3% yield) as yellow solid. LCMS (ES⁺): m/z 387.1 [M+H]⁺.

Synthesis of trans-3-[2-[(4-acetamidocyclohexyl)amino]-5-fluoro-pyrimidin-4-yl]benzoic acid (186-3). To a solution of trans-methyl 3-[2-[(4-acetamidocyclohexyl)amino]-5-fluoro-pyrimidin-4-yl]benzoate (186-2) (200 mg, 518 μmol, 1.00 eq) in THF (6 mL) and H₂O (2 mL) was added LiOH·H₂O (65.1 mg, 1.55 mmol, 3.00 eq) at 25 °C, the mixture was stirred at 50 °C for 2 h. LC-MS showed that the desired mass was detected. The reaction was concentrated under reduced pressure to remove THF. Then the remaining aqueous layer was acidified by HCl (1 M) to adjust pH to 2-3 and the it was extracted with EtOAc (10 mL*3), the combined organic layers were dried over Na₂SO₄, filtered and the filtrate was concentrated under reduced pressure to give the title compound (186-3) (180 mg, crude) as yellow solid. LCMS (ES⁺): m/z 373.2 [M+H]⁺.

Synthesis of trans-N-[4-[[5-fluoro-4-[3-[(3S)-3-hydroxypyrrolidine-1-carbonyl]phenyl]pyrimidin-2-yl]amino]cyclohexyl]acetamide (Compound 252). To a solution of trans-3-[2-[(4-acetamidocyclohexyl)amino]-5-fluoro-pyrimidin-4-yl]benzoic acid (180 mg, 483 μmol, 1.00 eq) in DMF (2 mL) was added DIEA (62.5 mg, 483 μmol, 84.2 μL, 1.00 eq) and HATU (276 mg, 725 μmol, 1.50 eq) at 25 °C, the mixture was stirred at 25 °C for 10 min. Then (3S)-pyrrolidin-3-ol (42.1 mg, 483 μmol, 39.1 μL, 1.00 eq) was added to above reaction mixture at 25 °C, the mixture was stirred at 40 °C for 20 min. LC-MS showed that the desired mass was detected. To the mixture was added H₂O (5 mL) and the mixture was extracted with EtOAc (5 mL*3). The combined organic layers were washed with brine (3 mL*3), dried over Na₂SO₄, filtered and the filtrate was concentrated in reduced pressure to give the crude product. This crude product was purified by prep-HPLC (FA) (column: Phenomenex luna C18 100*40 mm*3 um; mobile phase: [H₂O (0.2% FA)-ACN]; gradient: 10%-40% B over 8.0 min) to give the title compound (Compound 252) (88.4 mg, 199 μmol, 41.2% yield, 99.50% purity) as white solid. LCMS (ES⁺): m/z 442.2 [M+H]⁺.

### Example 187. Synthesis of trans-(S)-4-((5-fluoro-4-(3-(3-hydroxypyrrolidine-1-carbonyl)phenyl)pyrimidin-2-yl)amino)cyclohexane-1-carboxamide (Compound 259)

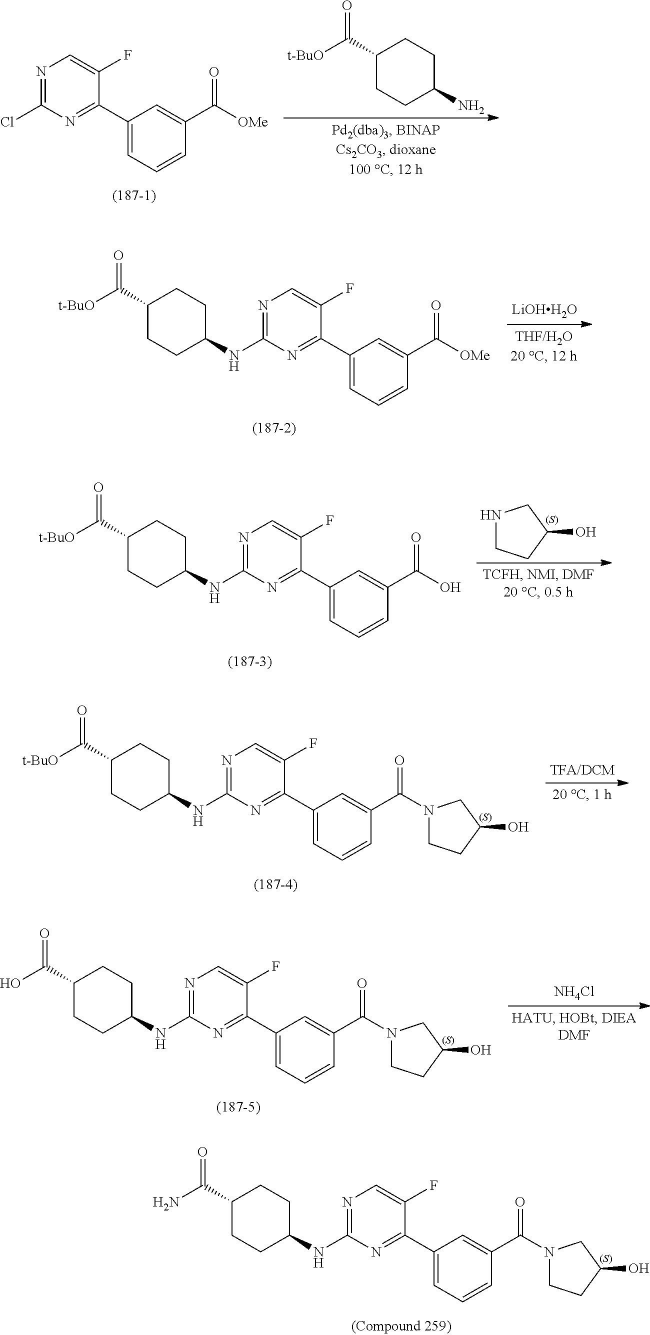

Synthesis of trans-methyl 3-(2-((4-(tert-butoxycarbonyl)cyclohexyl)amino)-5-fluoropyrimidin-4-yl)benzoate (187-2). To a solution of methyl 3-(2-chloro-5-fluoro-pyrimidin-4-yl)benzoate (187-1) (1.20 g, 4.50 mmol, 1.00 eq) and trans-tert-butyl 4-aminocyclohexanecarboxylate (1.79 g, 9.00 mmol, 2.00 eq) in dioxane (50 mL) were added Cs₂CO₃ (4.40 g, 13.50 mmol, 3.00 eq), BINAP (280 mg, 450 μmol, 0.100 eq) and Pd₂(dba)₃ (412 mg, 450 μmol, 0.100 eq) at 20 °C under N₂, the mixture was stirred at 100 °C for 12 h under N₂. LCMS showed methyl 3-(2-chloro-5-fluoro-pyrimidin-4-yl)benzoate (187-1) was consumed completely and the desired mass was detected. Water (50 mL) was added to the mixture, the mixture was extracted with EtOAc (50 mL*3), then the combined organic layers were dried over Na₂SO₄, filtered and the filtrate was concentrated under reduced pressure to give a residue. The residue was purified by column chromatography (SiO₂, Petroleum ether/Ethyl acetate=1/0 to 5/1) to give the title compound (187-2) (1.41 g, 3.28 mmol, 72.95% yield) as a yellow solid. LCMS (ES⁺): m/z 430.2 [M+H]⁺.

Synthesis of trans-3-(2-((4-(tert-butoxycarbonyl)cyclohexyl)amino)-5-fluoropyrimidin-4-yl)benzoic acid (187-3). To a solution of trans-methyl 3-(2-((4-(tert-butoxycarbonyl)cyclohexyl)amino)-5-fluoropyrimidin-4-yl)benzoate (187-2) (200 mg, 466 mol, 1.00 eq) in THF (4 mL) and H₂O (2 mL) was added LiOH·H₂O (78.2 mg, 1.86 mmol, 4.00 eq) at 20 °C, the mixture was stirred at 20 °C for 12 h. LCMS showed trans-methyl 3-(2-((4-(tert-butoxycarbonyl)cyclohexyl)amino)-5-fluoropyrimidin-4-yl)benzoate (187-2) was consumed completely and the desired mass was detected. The reaction mixture was concentrated under reduced pressure to remove THF. Then HCl (1 M) was added to the remaining aqueous phase to adjust pH to 4-5, a solid was precipitated, then filtered, the filter cake was dried under reduced pressure to give the title compound (187-3) (180 mg, crude) as a yellow solid. LCMS (ES⁺): m/z 416.2 [M+H]⁺.

Synthesis of trans-tert-butyl (S)-4-((5-fluoro-4-(3-(3-hydroxypyrrolidine-1-carbonyl)phenyl)pyrimidin-2-yl)amino)cyclohexane-1-carboxylate (187-4). To a solution of trans-3-[2-[(4-tert-butoxycarbonylcyclohexyl)amino]-5-fluoro-pyrimidin-4-yl]benzoic acid (187-3) (180 mg, 433 μmol, 1.00 eq) and (3S)-pyrrolidin-3-ol (37.8 mg, 433 μmol, 35.0 μL, 1.00 eq) in DMF (2 mL) were added TCFH (122 mg, 433 μmol, 1.00 eq) and NMI (71.1 mg, 867 μmol, 69.1 μL, 2.00 eq) at 20 °C, the mixture was stirred at 20 °C for 0.5 h. LCMS showed trans-3-[2-[(4-tert-butoxycarbonylcyclohexyl)amino]-5-fluoro-pyrimidin-4-yl]benzoic acid (187-3) was consumed completely and the desired mass was detected. Water (5 mL) was added to the mixture, then it was extracted with EtOAc (5 mL*3), the combined organic layers were dried over Na₂SO₄, filtered and the filtrate was concentrated under reduced pressure to give the title compound (187-4) (210 mg, crude) as a yellow oil. LCMS (ES⁺): m/z 485.2 [M+H]⁺.

Synthesis of trans-(S)-4-((5-fluoro-4-(3-(3-hydroxypyrrolidine-1-carbonyl)phenyl)pyrimidin-2-yl)amino)cyclohexane-1-carboxylic acid (187-5). A solution of trans-tert-butyl (S)-4-((5-fluoro-4-(3-(3-hydroxypyrrolidine-1-carbonyl)phenyl)pyrimidin-2-yl)amino)cyclohexane-1-carboxylate (187-4) (210 mg, 433 μmol, 1.00 eq) in TFA (0.5 mL) and DCM (1.5 mL) was stirred at 20 °C for 1 h. LCMS showed trans-tert-butyl (S)-4-((5-fluoro-4-(3-(3-hydroxypyrrolidine-1-carbonyl)phenyl)pyrimidin-2-yl)amino)cyclohexane-1-carboxylate (187-4) remained and the desired mass was detected. Then the mixture was stirred at 20 °C for another 12 h, LCMS showed trans-tert-butyl (S)-4-((5-fluoro-4-(3-(3-hydroxypyrrolidine-1-carbonyl)phenyl)pyrimidin-2-yl)amino)cyclohexane-1-carboxylate (187-4) was consumed completely and the desired mass was detected. The reaction mixture was concentrated under reduced pressure to give the title compound (187-5) (185 mg, crude) as a yellow oil. LCMS (ES⁺): m/z 429.2 [M+H]⁺.

Synthesis of trans-(S)-4-((5-fluoro-4-(3-(3-hydroxypyrrolidine-1-carbonyl)phenyl)pyrimidin-2-yl)amino)cyclohexane-1-carboxamide (Compound 259). To a solution of trans-(S)-4-((5-fluoro-4-(3-(3-hydroxypyrrolidine-1-carbonyl)phenyl)pyrimidin-2-yl)amino)cyclohexane-1-carboxylic acid (187-5) (185 mg, 173 μmol, 1.00 eq) and NH₄Cl (13.9 mg, 259 μmol, 1.50 eq) in DMF (0.5 mL) were added HATU (78.8 mg, 207 μmol, 1.20 eq), HOBt (11.7 mg, 86.4 μmol, 0.5000 eq) and DIEA (44.6 mg, 345 μmol, 60.2 μL, 2.00 eq) at 20 °C, the mixture was stirred at 20 °C for 12 h. LCMS showed trans-(S)-4-((5-fluoro-4-(3-(3-hydroxypyrrolidine-1-carbonyl)phenyl)pyrimidin-2-yl)amino)cyclohexane-1-carboxylic acid (187-5) was consumed completely and the desired mass was detected. The reaction was filtered and the filtrate was concentrated under reduced pressure to give a residue. The residue was purified by prep-HPLC (column: Phenomenex luna C18 100*40 mm*3 um; mobile phase: [H₂O (0.04% HCl)-ACN]; gradient: 10%-30% B over 18.0 min) to give the title compound (Compound 259) (30.0 mg, 69.0 μmol, 40.0% yield, 98.3% purity) as a yellow solid. LCMS (ES⁺): m/z 428.2 [M+H]⁺.

### Example 188. Synthesis of (R)-1-(4-((5-fluoro-4-(3-(3-hydroxypyrrolidine-1-carbonyl)phenyl)pyrimidin-2-yl)amino)piperidin-1-yl)ethan-1-one (Compound 213)

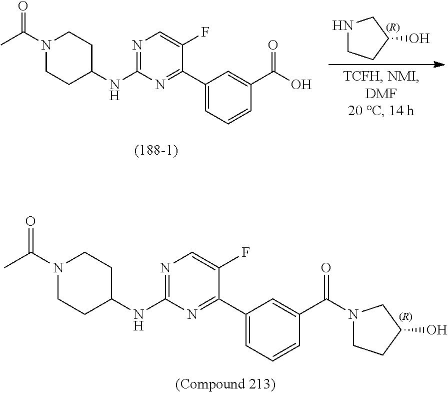

Synthesis of (R)-1-(4-((5-fluoro-4-(3-(3-hydroxypyrrolidine-1-carbonyl)phenyl)pyrimidin-2-yl)amino)piperidin-1-yl)ethan-1-one (Compound 213). To a solution of 3-(2-((1-acetylpiperidin-4-yl)amino)-5-fluoropyrimidin-4-yl)benzoic acid (188-1) (300 mg, 837 μmol, 1.00 eq) in DMF (5 mL) were added (3R)-pyrrolidin-3-ol (72.9 mg, 837 μmol, 69.6 μL, 1.00 eq), TCFH (517 mg, 1.84 mmol, 2.20 eq) and NMI (206 mg, 2.51 mmol, 200 μL, 3.00 eq) at 20 °C. The mixture was stirred at 20 °C for 12 h. LC-MS showed ~31% of 3-(2-((1-acetylpiperidin-4-yl)amino)-5-fluoropyrimidin-4-yl)benzoic acid (188-1) was remaining, and ~3% of the desired mass was detected. Then (3R)-pyrrolidin-3-ol (73.0 mg) and TCFH (160 mg) were added to the mixture at 20 °C. The mixture was stirred at 20 °C for another 2 h. LC-MS showed ~30% of 3-(2-((1-acetylpiperidin-4-yl)amino)-5-fluoropyrimidin-4-yl)benzoic acid (188-1) was remaining, and ~21% of the desired mass was detected. The reaction mixture was filtered and the filtrate was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 15%-45% B over 8.0 min) to give the title compound (Compound 213) (121 mg, 281 μmol, 33.5% yield, 98.9% purity) as a pink solid. LCMS (ES⁺): m/z 428.2 [M+H]⁺.

### Example 189. Synthesis of 1-(4-((5-fluoro-4-(3-(3-hydroxyazetidine-1-carbonyl)phenyl)pyrimidin-2-yl)amino)piperidin-1-yl)ethan-1-one (Compound 214)

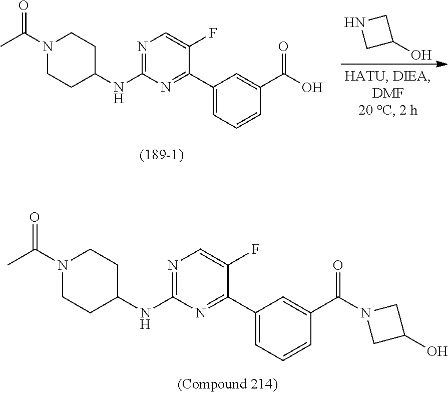 [SMILES: CC(=O)N1CCC(Nc2ncc(F)c(-c3cccc(C(=O)O)c3)n2)CC1 ; OC1CNC1 ; CC(=O)N1CCC(Nc2ncc(F)c(-c3cccc(C(=O)N4CC(O)C4)c3)n2)CC1]

Synthesis of 1-(4-((5-fluoro-4-(3-(3-hydroxyazetidine-1-carbonyl)phenyl)pyrimidin-2-yl)amino)piperidin-1-yl)ethan-1-one (Compound 214). To a stirred solution of 3-(2-((1-acetylpiperidin-4-yl)amino)-5-fluoropyrimidin-4-yl)benzoic acid (189-1) (300 mg, 837 μmol, 1.00 eq) in DMF (2 mL) were added DIEA (216 mg, 1.67 mmol, 292 μL, 2.00 eq), HATU (414 mg, 1.09 mmol, 1.30 eq) and azetidin-3-ol (61.2 mg, 837 μmol, 1.00 eq) at 20 °C, the resulting mixture was stirred at 20 °C for 2 h under N₂. LCMS showed that 3-(2-((1-acetylpiperidin-4-yl)amino)-5-fluoropyrimidin-4-yl)benzoic acid (189-1) was remaining little and the desired mass was detected. The mixture was concentrated under reduced pressure to give a residue. This residue was purified by prep-HPLC (column: Phenomenex Luna C18 100*30 mm*3 um; mobile phase: [H₂O (0.2% FA)-ACN]; gradient: 5%-40% B over 8.0 min) to give the title compound (Compound 214) (170 mg, 400 μmol, 47.8% yield, 97.3% purity) as white solid. LCMS (ES⁺): m/z 414.1 [M+H]⁺.

### Example 190. Synthesis of 1-[4-[[5-fluoro-4-[3-[4-hydroxy-4-(hydroxymethyl)piperidine-1-carbonyl]phenyl]pyrimidin-2-yl]amino]-1-piperidyl]ethanone (Compound 217)

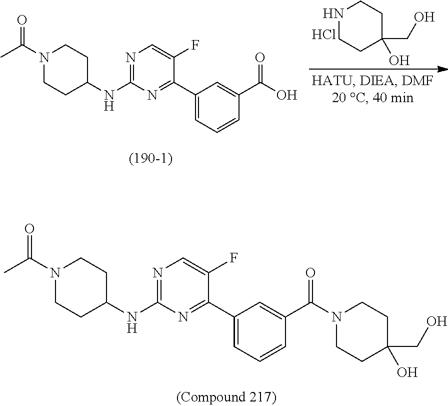 [SMILES: CC(=O)N1CCC(Nc2ncc(F)c(-c3cccc(C(=O)O)c3)n2)CC1 ; Cl ; OCC1(O)CCNCC1 ; CC(=O)N1CCC(Nc2ncc(F)c(-c3cccc(C(=O)N4CCC(O)(CO)CC4)c3)n2)CC1]

1-[4-[[5-fluoro-4-[3-[4-hydroxy-4-(hydroxymethyl)piperidine-1-carbonyl]phenyl]pyrimidin-2-yl]amino]-1-piperidyl]ethanone (Compound 217). To a solution of 3-[2-[(1-acetyl-4-piperidyl)amino]-5-fluoro-pyrimidin-4-yl]benzoic acid (190-1) (300 mg, 837 μmol, 1.00 eq) in DMF (3 mL) were added DIEA (216 mg, 1.67 mmol, 292 μL, 2.00 eq) and HATU (350 mg, 921 μmol, 1.10 eq) at 25 °C, the mixture was stirred at 25 °C for 10 min. Then 4-(hydroxymethyl)piperidin-4-ol (140 mg, 837 μmol, 1.00 eq, HCl) was added at 25 °C, the mixture was stirred at 25 °C for 30 min. LC-MS showed that the desired mass was detected. To the mixture was added H₂O (10 mL) and the mixture was extracted with EtOAc (10 mL*3). The combined organic layers were washed with brine (10 mL*3), dried over Na₂SO₄, filtered and the filtrate was concentrated in reduced pressure to give the crude product. This mixture was purified by prep-HPLC (column: Phenomenex luna C18 100*40 mm*5 um; mobile phase: [H₂O (0.2% FA)-ACN]; gradient: 10%-50% B over 8.0 min) to give the title compound (Compound 217) (220 mg, 447 μmol, 53.4% yield, 95.80% purity) as white solid. LCMS (ES⁺): m/z 472.1 [M+H]⁺.

### Example 191. Synthesis of 1-(4-((4-(3-(4-(aminomethyl)-4-hydroxypiperidine-1-carbonyl)phenyl)-5-fluoropyrimidin-2-yl)amino)piperidin-1-yl)ethan-1-one (Compound 218)

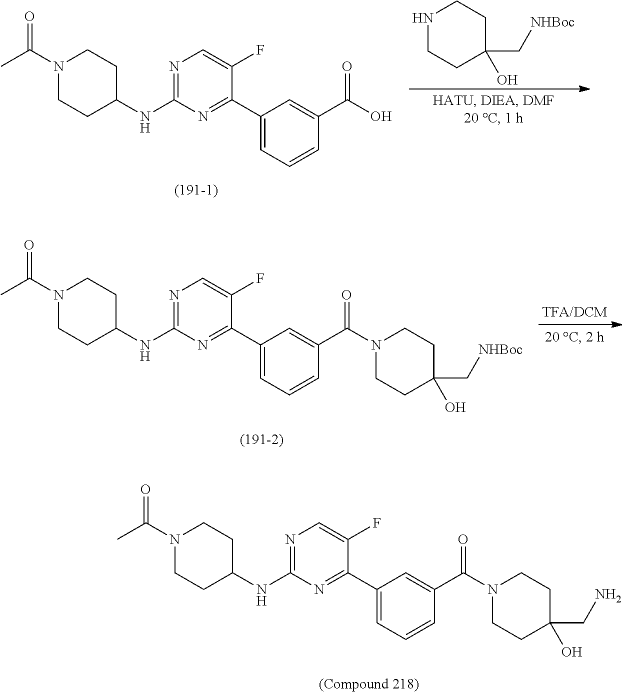 [SMILES: CC(=O)N1CCC(Nc2ncc(F)c(-c3cccc(C(=O)O)c3)n2)CC1 ; CC(C)(C)OC(=O)NCC1(O)CCNCC1 ; CC(=O)N1CCC(Nc2ncc(F)c(-c3cccc(C(=O)N4CCC(O)(CNC(=O)OC(C)(C)C)CC4)c3)n2)CC1 ; CC(=O)N1CCC(Nc2ncc(F)c(-c3cccc(C(=O)N4CCC(O)(CN)CC4)c3)n2)CC1]

Synthesis of tert-butyl ((1-(3-(2-((1-acetylpiperidin-4-yl)amino)-5-fluoropyrimidin-4-yl)benzoyl)-4-hydroxypiperidin-4-yl)methyl)carbamate (191-2). To a solution of 3-(2-((1-acetylpiperidin-4-yl)amino)-5-fluoropyrimidin-4-yl)benzoic acid (191-1) (300 mg, 837 μmol, 1.00 eq) in DMF (3 mL) were added HATU (382 mg, 1.00 mmol, 1.20 eq) and DIEA (325 mg, 2.51 mmol, 437 μL, 3.00 eq) at 20 °C, then tert-butyl ((4-hydroxypiperidin-4-yl)methyl)carbamate (193 mg, 837 μmol, 1.00 eq) was added to the mixture, the mixture was stirred at 20 °C for 1 h. LCMS showed 3-(2-((1-acetylpiperidin-4-yl)amino)-5-fluoropyrimidin-4-yl)benzoic acid (191-1) was consumed and the desired target MS was detected. H₂O (3 mL) was added to the solution, the mixture was extracted with EtOAc (3 mL*3), then the combined organic phase was washed with brine (3 mL*3), dried over Na₂SO₄, filtered and the filtrated was concentrated in vacuo to give the crude product. The crude product was purified by prep-TLC (SiO₂, EtOAc:MeOH=10:1) to give the title compound (191-2) (450 mg, 789 μmol, 94.2% yield) as a yellow oil. LCMS (ES⁺): m/z 571.4 [M+H]⁺.

Synthesis of 1-(4-((4-(3-(4-(aminomethyl)-4-hydroxypiperidine-1-carbonyl)phenyl)-5-fluoropyrimidin-2-yl)amino)piperidin-1-yl)ethan-1-one (Compound 218). A solution of ((1-(3-(2-((1-acetylpiperidin-4-yl)amino)-5-fluoropyrimidin-4-yl)benzoyl)-4-hydroxypiperidin-4-yl)methyl)carbamate (191-2) (450 mg, 789 μmol, 1.00 eq) in DCM (4 mL) and TFA (0.6 mL) was stirred at 20 °C for 2 h. LCMS showed ((1-(3-(2-((1-acetylpiperidin-4-yl)amino)-5-fluoropyrimidin-4-yl)benzoyl)-4-hydroxypiperidin-4-yl)methyl)carbamate (191-2) was consumed and the desired target MS was detected. The mixture was concentrated in vacuo to give a residue. The residue was purified by prep-HPLC (column: Waters Xbridge Prep OBD C18 150*40 mm*10 um; mobile phase: [H₂O (10 mM NH₄HCO₃)-ACN]; gradient: 5%-35% B over 8.0 min) to give the title compound (Compound 218) (54.1 mg, 91.8 μmol, 11.6% yield, 99.2% purity, TFA) as a white solid. LCMS (ES⁺): m/z 471.2 [M+H]⁺.

### Example 192. Synthesis of 1-(3-(2-((1-acetylpiperidin-4-yl)amino)-5-fluoropyrimidin-4-yl)phenyl)-3-hydroxypyrrolidin-2-one (Compound 228)

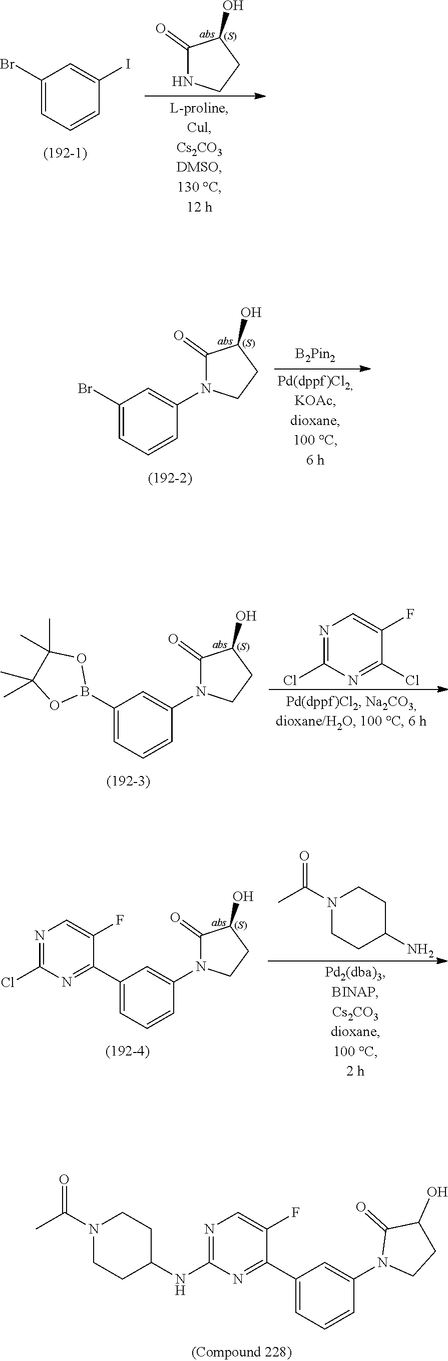

Synthesis of (S)-1-(3-bromophenyl)-3-hydroxypyrrolidin-2-one (192-2). A mixture of 1-bromo-3-iodo-benzene (192-1) (10.0 g, 35.3 mmol, 4.51 mL, 1.00 eq), (3S)-3-hydroxypyrrolidin-2-one (3.93 g, 38.8 mmol, 1.10 eq), CuI (673 mg, 3.53 mmol, 0.100 eq), Cs₂CO₃ (34.5 g, 106 mmol, 3.00 eq) and L-PROLINE (406 mg, 3.53 mmol, 0.100 eq) in DMSO (50 mL) at 25 °C was degassed and purged with N₂ for 3 times, and then the mixture was stirred at 130 °C for 12 h under N₂ atmosphere. LC-MS showed 1-bromo-3-iodo-benzene (192-1) was consumed completely and the desired mass was detected. The reaction diluted with H₂O 50 mL and combined organic layers were extracted with EtOAc (70 mL*3), and concentrated under reduced pressure. The residue was purified by column chromatography (SiO₂, Petroleum ether:Ethyl acetate=1:0 to 0:1) to give the title compound (192-2) (4.20 g, 14.4 mmol, 40.8% yield, 87.9% purity) as off white solid. LCMS (ES⁺): m/z 256.0 [M+H]⁺.

Synthesis of (S)-3-hydroxy-1-(3-(4,4,5,5-tetramethyl-1,3,2-dioxaborolan-2-yl)phenyl)pyrrolidin-2-one (192-3). A mixture of (S)-1-(3-bromophenyl)-3-hydroxypyrrolidin-2-one (192-2) (2.50 g, 9.76 mmol, 1.00 eq), 4,4,5,5-tetramethyl-2-(4,4,5,5-tetramethyl-1,3,2-dioxaborolan-2-yl)-1,3,2-dioxaborolane (2.48 g, 9.76 mmol, 1.00 eq), KOAc (2.87 g, 29.2 mmol, 3.00 eq), Pd(dppf)Cl₂ (1.43 g, 1.95 mmol, 0.200 eq) in dioxane (2 mL) at 25 °C was degassed and purged with N₂ for 3 times, and then the mixture was stirred at 100 °C for 6 h under N₂ atmosphere. LC-MS showed (S)-1-(3-bromophenyl)-3-hydroxypyrrolidin-2-one (192-2) was consumed completely and the desired mass was detected. The reaction diluted with H₂O 30 mL and combined organic layers were extracted with EtOAc (50 mL*3), and concentrated under reduced pressure. The residue was purified by column chromatography (SiO₂, Petroleum ether:Ethyl acetate=1:0 to 0:1) to give the title compound (192-3) (1.70 g, 5.09 mmol, 52.1% yield, 90.7% purity) as a brown solid. LCMS (ES⁺): m/z 304.2 [M+H]⁺.

Synthesis of (S)-1-(3-(2-chloro-5-fluoropyrimidin-4-yl)phenyl)-3-hydroxypyrrolidin-2-one (192-4). A mixture of (S)-3-hydroxy-1-(3-(4,4,5,5-tetramethyl-1,3,2-dioxaborolan-2-yl)phenyl)pyrrolidin-2-one (192-3) (1.70 g, 5.61 mmol, 1.00 eq), 2,4-dichloro-5-fluoro-pyrimidine (936 mg, 5.61 mmol, 1.00 eq), Na₂CO₃ (1.78 g, 16.8 mmol, 3.00 eq), Pd(dppf)Cl₂ (820 mg, 1.12 mmol, 0.200 eq) in dioxane (15 mL) and H₂O (2 mL) at 25 °C was degassed and purged with N₂ for 3 times, and then the mixture was stirred at 100 °C for 6 h under N₂ atmosphere. LC-MS showed (S)-3-hydroxy-1-(3-(4,4,5,5-tetramethyl-1,3,2-dioxaborolan-2-yl)phenyl)pyrrolidin-2-one (192-3) was consumed completely and the desired mass was detected. The reaction mixture was diluted with H₂O 30 mL and extracted with EtOAc (50 mL*3). The combined organic layers were concentrated under reduced pressure. The residue was purified by column chromatography (SiO₂, Petroleum ether:Ethyl acetate=1:0 to 1:1) to give the title compound (192-4) (1.70 g, 5.41 mmol, 96.5% yield, 98.0% purity) as a brown solid. LCMS (ES⁺): m/z 308.1 [M+H]⁺.

Synthesis of 1-(3-(2-((1-acetylpiperidin-4-yl)amino)-5-fluoropyrimidin-4-yl)phenyl)-3-hydroxypyrrolidin-2-one (Compound 228). A mixture of (S)-1-(3-(2-chloro-5-fluoropyrimidin-4-yl)phenyl)-3-hydroxypyrrolidin-2-one (192-4) (200 mg, 649 μmol, 1.00 eq), 1-(4-amino-1-piperidyl)ethanone (92.4 mg, 649 μmol, 1.00 eq), BINAP (80.9 mg, 129 μmol, 0.200 eq), Pd₂(dba)₃ (119 mg, 129 μmol, 0.200 eq) and Cs₂CO₃ (635 mg, 1.95 mmol, 3.00 eq) in dioxane (10 mL) was degassed and purged with N₂ for 3 times, and then the mixture was stirred at 100 °C for 2 h under N₂ atmosphere. LC-MS showed (S)-1-(3-(2-chloro-5-fluoropyrimidin-4-yl)phenyl)-3-hydroxypyrrolidin-2-one (192-4) was consumed completely and the desired mass was detected. The reaction diluted with H₂O 20 mL and combined organic layers were extracted with EtOAc (40 mL*3), and concentrated under reduced pressure. The residue was purified by prep-HPLC (column: Phenomenex luna C18 100*40 mm*5 um; mobile phase: [H₂O (0.2% FA)-ACN]; gradient: 10%-50% B over 8.0 min) to give the title compound (Compound 228) (19.2 mg, 45.1 μmol, 6.95% yield, 97.2% purity) as off white solid. LCMS (ES⁺): m/z 414.2 [M+H]⁺.

### Example 193. Synthesis of 1-(4-((5-fluoro-4-((S)-3-((S)-3-hydroxypyrrolidine-1-carbonyl)piperidin-1-yl)pyrimidin-2-yl)amino)piperidin-1-yl)ethan-1-one (Compound 215)

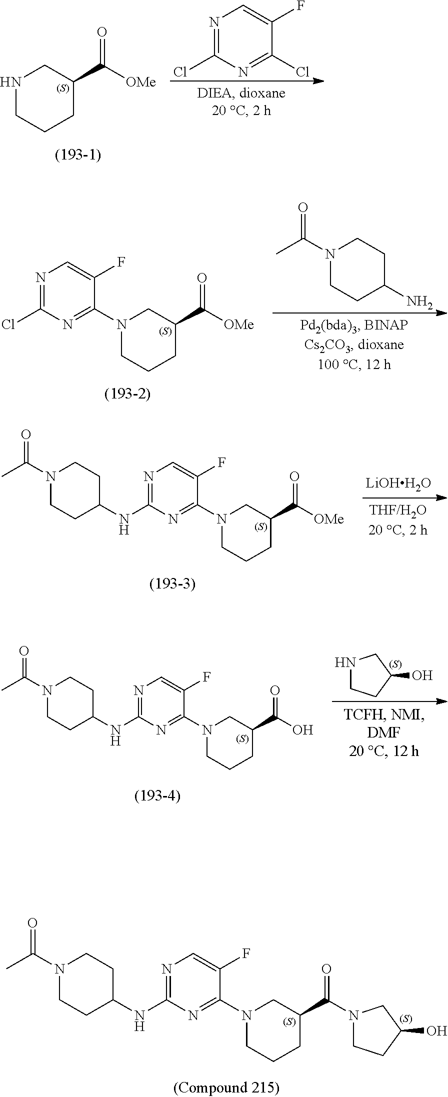

Synthesis of methyl (S)-1-(2-chloro-5-fluoropyrimidin-4-yl)piperidine-3-carboxylate (193-2). To a solution of methyl (S)-piperidine-3-carboxylate (193-1) (4.50 g, 25.1 mmol, 1.00 eq, HCl) in dioxane (50 mL) were added 2,4-dichloro-5-fluoropyrimidine (4.18 g, 25.1 mmol, 1.00 eq) and DIEA (9.71 g, 75.2 mmol, 13.1 mL, 3.00 eq) at 20 °C. The mixture was stirred at 20 °C for 2 h. LCMS showed the desired mass was detected. The mixture was diluted with H₂O (100 mL) and then extracted with DCM (70 mL*3). The combined organic layers were dried over Na₂SO₄, filtered and the filtrate was concentrated under reduced pressure to give the title compound (193-2) (6.26 g, crude) as a yellow oil. LCMS (ES⁺): m/z 274.0 [M+H]⁺.

Synthesis of methyl (S)-1-(2-((1-acetylpiperidin-4-yl)amino)-5-fluoropyrimidin-4-yl)piperidine-3-carboxylate (193-3). To a solution of methyl (S)-1-(2-chloro-5-fluoropyrimidin-4-yl)piperidine-3-carboxylate (193-2) (6.26 g, 22.9 mmol, 1.00 eq) and 1-(4-aminopiperidin-1-yl)ethan-1-one (3.25 g, 22.9 mmol, 1.00 eq) in dioxane (100 mL) were added Cs₂CO₃ (14.9 g, 45.8 mmol, 2.00 eq), BINAP (855 mg, 1.37 mmol, 0.06 eq) and Pd₂(dba)₃ (628 mg, 686 μmol, 0.03 eq) at 20 °C under N₂ atmosphere, the mixture was stirred at 100 °C for 12 h under N₂ atmosphere. LCMS showed methyl (S)-1-(2-chloro-5-fluoropyrimidin-4-yl)piperidine-3-carboxylate (193-2) was consumed and the desired mass was detected. The mixture was diluted with H₂O (100 mL) and then extracted with EtOAc (60 mL*3). The combined organic layers were dried over Na₂SO₄, filtered and the filtrate was concentrated under reduced pressure. The residue was purified by column chromatography (SiO₂, EtOAc=1/0 to 10/1) to give the title compound (193-3) (5.53 g, 14.6 mmol, 63.7% yield) as a white solid. LCMS (ES⁺): m/z 380.1 [M+H]⁺.

Synthesis of (S)-1-(2-((1-acetylpiperidin-4-yl)amino)-5-fluoropyrimidin-4-yl)piperidine-3-carboxylic acid (193-4). To a solution of methyl (S)-1-(2-((1-acetylpiperidin-4-yl)amino)-5-fluoropyrimidin-4-yl)piperidine-3-carboxylate (193-3) (5.53 g, 14.6 mmol, 1.00 eq) in THF (21 mL) and H₂O (7 mL) was added LiOH·H₂O (1.22 g, 29.2 mmol, 2.00 eq) at 20 °C. The mixture was stirred at 20 °C for 2 h. LCMS showed methyl (S)-1-(2-((1-acetylpiperidin-4-yl)amino)-5-fluoropyrimidin-4-yl)piperidine-3-carboxylate (193-3) was consumed and the desired mass was detected. The mixture was concentrated under reduced pressure to remove THF, then HCl (1M) was added to the aqueous phase to adjusted pH to 5, the precipitated solid was filtered and the filter cake was dried in vacuo to give the title compound (193-4) (5.30 g, crude) as a yellow solid. LCMS (ES⁺): m/z 366.3 [M+H]+.

Synthesis of 1-(4-((5-fluoro-4-((S)-3-((S)-3-hydroxypyrrolidine-1-carbonyl)piperidin-1-yl)pyrimidin-2-yl)amino)piperidin-1-yl)ethan-1-one (Compound 215). To a solution of (S)-1-(2-((1-acetylpiperidin-4-yl)amino)-5-fluoropyrimidin-4-yl)piperidine-3-carboxylic acid (193-4) (200 mg, 547 μmol, 1.00 eq) in DMF (1 mL) were added TCFH (184 mg, 657 μmol, 1.20 eq) and NMI (135 mg, 1.64 mmol, 131 μL, 3.00 eq) at 20 °C, then (S)-pyrrolidin-3-ol (47.7 mg, 547 μmol, 44.23 μL, 1.00 eq) was added to the mixture, the mixture was stirred at 20 °C for 12 h. LCMS showed (S)-1-(2-((1-acetylpiperidin-4-yl)amino)-5-fluoropyrimidin-4-yl)piperidine-3-carboxylic acid (193-4) was consumed and the desired mass was detected. The reaction mixture was concentrated under reduced pressure to remove solvent to give a residue. The residue was purified by prep-HPLC (column: Phenomenex luna C18 100*40 mm*3 um; mobile phase: [H₂O (0.2% FA)-ACN]; gradient: 1%-35% B over 8.0 min) to give the title compound (Compound 215) (96.6 mg, 201 μmol, 36.7% yield, 100% purity, FA) as a white solid. LCMS (ES⁺): m/z 435.2 [M+H]⁺.

### Example 194. Synthesis of 1-(4-((5-fluoro-4-((S)-3-((R)-3-hydroxypyrrolidine-1-carbonyl)piperidin-1-yl)pyrimidin-2-yl)amino)piperidin-1-yl)ethan-1-one (Compound 216)

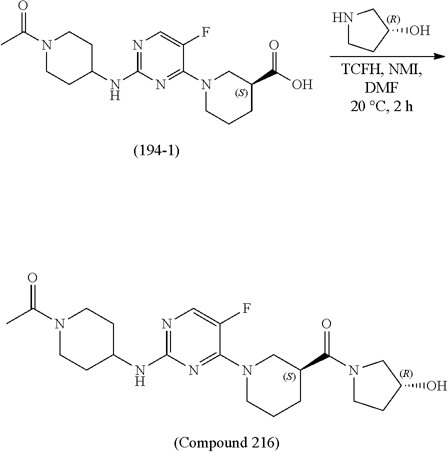

Synthesis of 1-(4-((5-fluoro-4-((S)-3-((R)-3-hydroxypyrrolidine-1-carbonyl)piperidin-1-yl)pyrimidin-2-yl)amino)piperidin-1-yl)ethan-1-one (Compound 216). To a solution of (S)-1-(2-((1-acetylpiperidin-4-yl)amino)-5-fluoropyrimidin-4-yl)piperidine-3-carboxylic acid (194-1) (300 mg, 821 μmol, 1.00 eq) in DMF (3 mL) were added TCFH (507 mg, 1.81 mmol, 2.20 eq) and NMI (87.6 mg, 1.07 mmol, 85.1 μL, 1.30 eq) at 20 °C, then (R)-pyrrolidin-3-ol (107 mg, 1.23 mmol, 102 μL, 1.50 eq) was added to the mixture, the mixture was stirred at 20 °C for 1 h. LCMS showed (S)-1-(2-((1-acetylpiperidin-4-yl)amino)-5-fluoropyrimidin-4-yl)piperidine-3-carboxylic acid (194-1) remained and the desired target MS was detected. Then (R)-pyrrolidin-3-ol (107 mg, 1.23 mmol, 1.50 eq) was added to the mixture, the mixture was stirred at 20 °C for another 1 h, LCMS showed (S)-1-(2-((1-acetylpiperidin-4-yl)amino)-5-fluoropyrimidin-4-yl)piperidine-3-carboxylic acid (194-1) was consumed and the desired target MS was detected. The mixture was concentrated in vacuo to give a residue. The residue was purified by prep-HPLC (column: Phenomenex Gemini-NX 80*40 mm*3 um; mobile phase: [H₂O (10 mM NH₄HCO₃)-ACN]; gradient: 2%-30% B over 8.0 min) to give the title compound (Compound 216) (88.0 mg, 202 μmol, 24.64% yield, 99.9% purity) as a white solid. LCMS (ES⁺): m/z 435.2 [M+H]⁺.

### Example 195. Synthesis of trans-(1r,4r)-4-((5-fluoro-4-(3-(2-oxopyridin-1 (2H)-yl)phenyl)pyrimidin-2-yl)amino)cyclohexane-1-carboxamide (Compound 240)

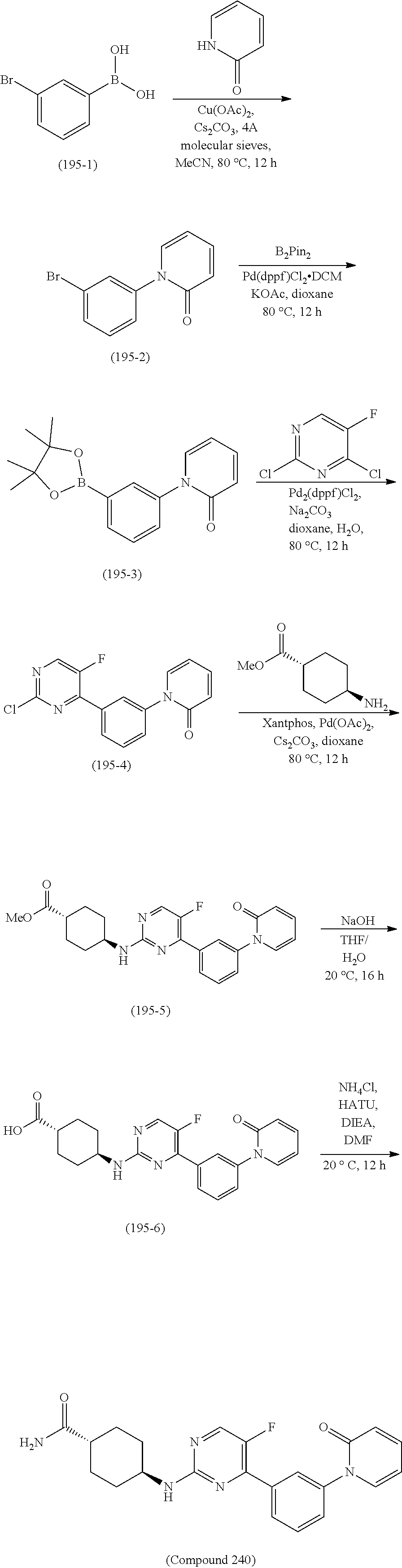

Synthesis of 1-(3-bromophenyl)pyridin-2(1H)-one (195-2). To a solution of (3-bromophenyl)boronic acid (195-1) (2.84 g, 29.9 mmol, 1.20 eq) in ACN (100 mL) were added pyridin-2(1H)-one (2.84 g, 29.9 mmol, 1.20 eq), Cu(OAc)₂ (2.26 g, 12.5 mmol, 0.500 eq), Cs₂CO₃ (16.2 g, 49.7 mmol, 2.00 eq) and 4 A MS (200 mg) at 20 °C. The mixture was stirred at 80 °C for 12 h under O₂ (15 psi). LC-MS showed that To a solution of (3-bromophenyl)boronic acid (195-1) was consumed completely and one main peak with the desired mass was detected. The reaction mixture was diluted with DCM (50 mL) and filtered, the filtrate was concentrated under reduced pressure. The crude product was triturated with MeOH (20 ml) and filtered, the filter cake was dried in a vacuum to give the title compound (195-2) (2.50 g, 10.0 mmol, 40.2% yield, n/a purity) as a white solid. LCMS (ES⁺): m/z 250.1 [M+H]⁺.

Synthesis of 1-(3-(4,4,5,5-tetramethyl-1,3,2-dioxaborolan-2-yl)phenyl)pyridin-2 (1H)-one (195-3). To a solution of 1-(3-bromophenyl)pyridin-2(1H)-one (195-2) (5.08 g, 20.0 mmol, 2.00 eq) in dioxane (40.0 mL) was added 4,4,5,5-tetramethyl-2-(4,4,5,5-tetramethyl-1,3,2-dioxaborolan-2-yl)-1,3,2-dioxaborolane (5.08 g, 20.0 mmol, 2.00 eq) and KOAc (1.96 g, 20.0 mmol, 2.00 eq), Pd(dppf)Cl₂·CH₂Cl₂ (816 mg, 999 μmol, 0.100 eq) at 20 °C. The mixture was stirred at 80 °C for 12 h under N₂. LC-MS showed that 1-(3-bromophenyl)pyridin-2(1H)-one (195-2) was consumed completely and one main peak with the desired mass was detected. The mixture was filtered and the mixture was concentrated under reduced pressure. The residue was purified by column chromatography (SiO₂, Petroleum ether:Ethyl acetate=1:0 to 0:1) to give the title compound (195-3) (2.50 g, 8.41 mmol, 84.2% yield, n/a purity) as a brown oil. LCMS (ES⁺): m/z 298.2 [M+H]⁺.

Synthesis of 1-(3-(2-chloro-5-fluoropyrimidin-4-yl)phenyl)pyridin-2(1H)-one (195-4). To a solution of 1-(3-(4,4,5,5-tetramethyl-1,3,2-dioxaborolan-2-yl)phenyl)pyridin-2 (1H)-one (195-3) (2.50 g, 8.41 mmol, 1.00 eq) in dioxane (20.0 mL), H₂O (4.00 mL) was added 2,4-dichloro-5-fluoropyrimidine (1.69 g, 10.1 mmol, 1.20 eq), Pd(dppf)Cl₂ (616 mg, 842 μmol, 0.100 eq) and Na₂CO₃ (1.78 g, 16.8 mmol, 2.00 eq) at 20 °C. The mixture was stirred at 80 °C for 12 h under N₂. LC-MS showed that 1-(3-(4,4,5,5-tetramethyl-1,3,2-dioxaborolan-2-yl)phenyl)pyridin-2(1H)-one (195-3) was consumed completely and one main peak with the desired mass was detected. The mixture was filtered and the filtrate was concentrated under reduced pressure. The residue was purified by column chromatography (SiO₂, Petroleum ether:Ethyl acetate=1:0 to 0:1) to give the title compound (195-4) (2.50 g, 8.29 mmol, 98.5% yield, n/a purity) as a brown solid. LCMS (ES⁺): m/z 302.1 [M+H]⁺.

Synthesis of trans-methyl (1r,4r)-4-((5-fluoro-4-(3-(2-oxopyridin-1 (2H)-yl)phenyl)pyrimidin-2-yl)amino)cyclohexane-1-carboxylate (195-5). To a solution of 1-(3-(2-chloro-5-fluoropyrimidin-4-yl)phenyl)pyridin-2(1H)-one (195-4) (2.50 g, 8.29 mmol, 1.00 eq) in dioxane (20 mL) were added methyl trans-(1r,4r)-4-aminocyclohexane-1-carboxylate (1.30 g, 8.29 mmol, 1.00 eq), Xantphos (479 mg, 828 μmol, 9.99e-2 eq), Pd(OAc)₂ (186 mg, 828 μmol, 0.100 eq) and Cs₂CO₃ (5.40 g, 16.6 mmol, 2.00 eq) at 20 °C. The mixture was stirred at 80 °C for 12 h under N₂. LC-MS showed that 1-(3-(2-chloro-5-fluoropyrimidin-4-yl)phenyl)pyridin-2(1H)-one (195-4) was consumed completely and one main peak with the desired mass was detected. The mixture was filtered and the filtrate was concentrated under reduced pressure. The residue was purified by column chromatography (SiO₂, Petroleum ether/Ethyl acetate=1/0 to 0/1) to give the title compound (195-5) (1.80 g, 4.26 mmol, 51.4% yield, n/a purity) as a yellow solid. LCMS (ES⁺): m/z 423.2 [M+H]⁺.

Synthesis of trans-(1r,4r)-4-((5-fluoro-4-(3-(2-oxopyridin-1 (2H)-yl)phenyl)pyrimidin-2-yl)amino)cyclohexane-1-carboxylic acid (195-6). To a solution of trans-methyl (1r,4r)-4-((5-fluoro-4-(3-(2-oxopyridin-1 (2H)-yl)phenyl)pyrimidin-2-yl)amino)cyclohexane-1-carboxylate (195-5) (1.80 g, 4.26 mmol, 1.00 eq) in THE (20 mL) and H₂O (5 mL) was added NaOH (511 mg, 12.8 mmol, 3.00 eq) at 20 °C. The mixture was stirred at 20 °C for 12 h. LC-MS showed trans-methyl (1r,4r)-4-((5-fluoro-4-(3-(2-oxopyridin-1 (2H)-yl)phenyl)pyrimidin-2-yl)amino)cyclohexane-1-carboxylate (195-5) was not consumed completely and one peak with the desired mass was detected. The mixture was stirred at 20 °C for 4 h. LC-MS showed that methyl (1r,4r)-4-((5-fluoro-4-(3-(2-oxopyridin-1 (2H)-yl)phenyl)pyrimidin-2-yl)amino)cyclohexane-1-carboxylate (195-5) was not consumed completely and one main peak with the desired mass was detected. The mixture was concentrated under reduced pressure. The reaction mixture was diluted with H₂O (20 mL) and extracted with EtOAc (20 mL*5). The combined water layers were adjusted pH to 5, and extracted with EtOAc (20 mL*5), the combined organic layers were washed with brine (10 ml), dried over Na₂SO₄, filtered and concentrated under reduced pressure to give the title compound (195-6) (1.50 g, 3.67 mmol, 86.2% yield) as a yellow oil. LCMS (ES⁺): m/z 409.2 [M+H]⁺.

Synthesis of trans-(1r,4r)-4-((5-fluoro-4-(3-(2-oxopyridin-1 (2H)-yl)phenyl)pyrimidin-2-yl)amino)cyclohexane-1-carboxamide (Compound 240). To a solution of trans-(1r,4r)-4-((5-fluoro-4-(3-(2-oxopyridin-1 (2H)-yl)phenyl)pyrimidin-2-yl)amino)cyclohexane-1-carboxylic acid (195-6) (300 mg, 735 μmol, 1.00 eq) in DMF (3 mL) was added NH₄Cl (157 mg, 2.94 mmol, 4.00 eq) and DIEA (475 mg, 3.67 mmol, 640 L, 5.00 eq), HATU (838 mg, 2.20 mmol, 3.00 eq) at 20 °C. The mixture was stirred at 20 °C for 12 h. LC-MS showed trans-(1r,4r)-4-((5-fluoro-4-(3-(2-oxopyridin-1 (2H)-yl)phenyl)pyrimidin-2-yl)amino)cyclohexane-1-carboxylic acid (195-6) was consumed completely and one main peak with the desired mass was detected. The reaction mixture was purified by prep-HPLC (column: Phenomenex luna C18 100*40 mm*3 um; mobile phase: [H₂O (0.2% FA)-ACN]; gradient: 7%-37% B over 8.0 min) to give the title compound (Compound 240) (137 mg, 334 μmol, 45.5% yield, 99.4% purity) as a white solid. LCMS (ES⁺): m/z 408.1 [M+H]⁺.

### Example 196. Synthesis of trans-N-[4-[[5-fluoro-4-[3-(2-oxo-1-pyridyl)phenyl]pyrimidin-2-yl]amino]cyclohexyl]acetamide (Compound 238)

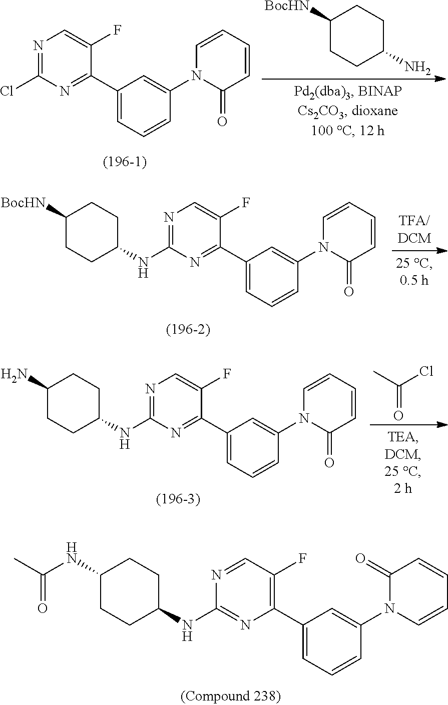

Synthesis of trans-tert-butyl (4-((5-fluoro-4-(3-(2-oxopyridin-1 (2H)-yl)phenyl)pyrimidin-2-yl)amino)cyclohexyl)carbamate (196-2). To a solution of 1-(3-(2-chloro-5-fluoropyrimidin-4-yl)phenyl)pyridin-2(1H)-one (196-1) (1.00 g, 3.31 mmol, 1.00 eq) and trans-tert-butyl (4-aminocyclohexyl)carbamate (1.07 g, 4.97 mmol, 1.50 eq) in dioxane (40 mL) were added BINAP (206 mg, 331 μmol, 0.100 eq), Cs₂CO₃ (3.24 g, 9.94 mmol, 3.00 eq) and Pd₂(dba)₃ (304 mg, 331 μmol, 0.100 eq) at 25 °C under N₂. The mixture was stirred at 100 °C for 12 h under N₂. LCMS showed 1-(3-(2-chloro-5-fluoropyrimidin-4-yl)phenyl)pyridin-2(1H)-one (196-1) was consumed completely and the desired mass was detected. The mixture was diluted with H₂O (50 mL) and then extracted with EtOAc (30 mL*3). The combined organic layers were dried over Na₂SO₄, filtered and the filtrate was concentrated under reduced pressure to give a residue. The residue was purified by MPLC (SiO₂, Petroleum ether/Ethyl acetate=1/0 to 0/1) to give the title compound (196-2) (1.08 g, 2.25 mmol, 68.0% yield) as a yellow oil. LCMS (ES⁺): m/z 480.3 [M+H]⁺.

Synthesis of trans-1-(3-(2-((4-aminocyclohexyl)amino)-5-fluoropyrimidin-4-yl)phenyl)pyridin-2(1H)-one (196-3). A solution of trans-tert-butyl (4-((5-fluoro-4-(3-(2-oxopyridin-1 (2H)-yl)phenyl)pyrimidin-2-yl)amino)cyclohexyl)carbamate (196-2) (700 mg, 1.46 mmol, 1.00 eq) in DCM (6 mL) and TFA (2 mL) was stirred at 25 °C for 0.5 h. LCMS showed trans-tert-butyl (4-((5-fluoro-4-(3-(2-oxopyridin-1 (2H)-yl)phenyl)pyrimidin-2-yl)amino)cyclohexyl)carbamate (196-2) was consumed completely and the desired mass was detected. The mixture was concentrated under reduced pressure to give the title compound (196-3) (700 mg, crude, TFA) as a yellow oil. LCMS (ES⁺): m/z 380.2 [M+H]⁺.

Synthesis of trans-N-[4-[[5-fluoro-4-[3-(2-oxo-1-pyridyl)phenyl]pyrimidin-2-yl]amino]cyclohexyl]acetamide (Compound 196). To a solution of trans-1-[3-[2-[(4-aminocyclohexyl)amino]-5-fluoro-pyrimidin-4-yl]phenyl]pyridin-2-one (196-3) (800 mg, 1.92 mmol, 1.00 eq, HCl) in DCM (10 mL) was added TEA (583 mg, 5.77 mmol, 803 μL, 3.00 eq) and acetyl chloride (166 mg, 2.12 mmol, 150 μL, 1.10 eq) at 25 °C. The mixture was stirred at 25 °C for 2 h. LC-MS showed trans-1-[3-[2-[(4-aminocyclohexyl)amino]-5-fluoro-pyrimidin-4-yl]phenyl]pyridin-2-one (196-3) was consumed completely and the desired mass was detected. The reaction mixture was diluted with water 10 mL and extracted with EtOAc (10 mL*3). The combined organic layers were washed with brine (10 mL*3), dried over Na₂SO₄, filtered and concentrated under reduced pressure. The residue was purified by prep-HPLC (column: Phenomenex Luna C18 100*30 mm*3 um; mobile phase: [H₂O (0.2% FA)-ACN]; gradient: 15%-55% B over 8.0 min) to give the title compound (Compound 196) (115 mg, 273 μmol, 14.2% yield, 100.00% purity) as a white solid. LCMS (ES⁺): m/z 422.2 [M+H]⁺.

### Example 197. Synthesis of 1-(3-(2-((1-acetylpiperidin-4-yl)amino)-5-fluoropyrimidin-4-yl)phenyl)pyridin-2(1H)-one (Compound 614)

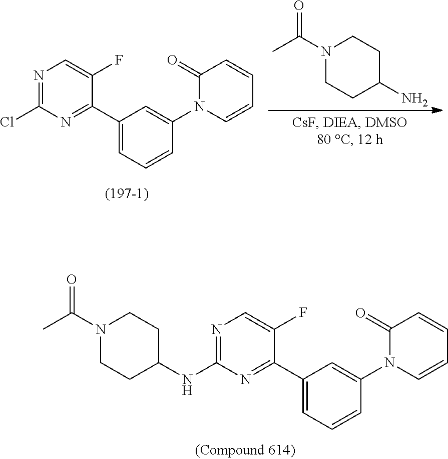

Synthesis of 1-(3-(2-((1-acetylpiperidin-4-yl)amino)-5-fluoropyrimidin-4-yl)phenyl)pyridin-2(1H)-one (Compound 614). To a solution of 1-[3-(2-chloro-5-fluoro-pyrimidin-4-yl)phenyl]pyridin-2-one (197-1) (200 mg, 662 μmol, 1.00 eq) and 1-(4-amino-1-piperidyl)ethanone (113 mg, 795 μmol, 1.20 eq) in DMSO (3 mL) was added CsF (201 mg, 1.33 mmol, 48.9 μL, 2.00 eq) and DIEA (171 mg, 1.33 mmol, 230 μL, 2.00 eq) at 25 °C. The mixture was stirred at 80 °C for 12 h. LC-MS showed the desired mass was detected. The reaction mixture was filtered and concentrated under reduced pressure. The residue was purified by prep-HPLC (column: Phenomenex luna C18 100*40 mm*3 um; mobile phase: [H₂O (0.2% FA)-ACN]; gradient: 10%-40% B over 8.0 min) to give the title compound (Compound 614) (146 mg, 359 μmol, 54.2% yield, 99.8% purity) as a pale yellow solid. LCMS (ES⁺): m/z 408.2 [M+H]⁺

### Example 198. Synthesis of (S)-1-(3-(2-((1-acetylpyrrolidin-3-yl)amino)-5-fluoropyrimidin-4-yl)phenyl)pyridin-2(1H)-one (Compound 613)

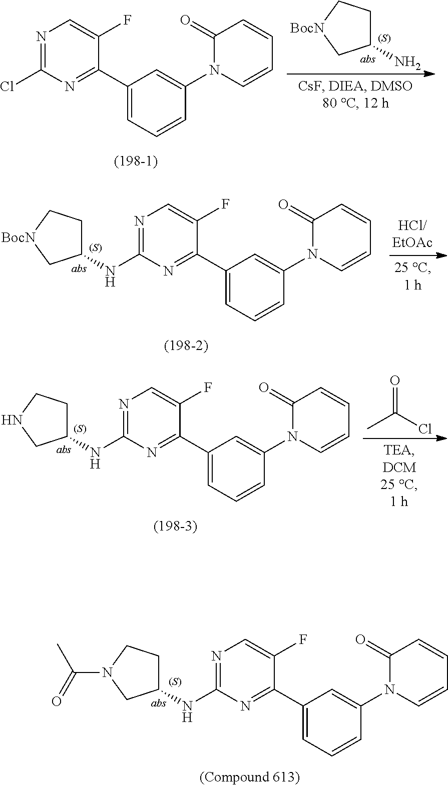

Synthesis of tert-butyl (S)-3-((5-fluoro-4-(3-(2-oxopyridin-1 (2H)-yl)phenyl)pyrimidin-2-yl)amino)pyrrolidine-1-carboxylate (198-2). To a solution of 1-[3-(2-chloro-5-fluoro-pyrimidin-4-yl)phenyl]pyridin-2-one (198-1) (4.00 g, 13.2 mmol, 1.00 eq) and tert-butyl (S)-3-aminopyrrolidine-1-carboxylate (2.96 g, 15.9 mmol, 1.20 eq) in DMSO (50 mL) was added CsF (4.03 g, 26.5 mmol, 978 μL, 2.00 eq) and DIEA (3.43 g, 26.5 mmol, 4.62 mL, 2.00 eq) at 20 °C. The mixture was stirred at 80 °C for 12 h. LC-MS showed the desired mass was detected. The reaction mixture was diluted with H₂O 30 mL and extracted with EtOAc 30 mL*3. The combined organic layers were washed with sat.aq. NaCl (10 mL), dried over Na₂SO₄, filtered and concentrated under reduced pressure. The residue was purified by column chromatography (SiO₂, Petroleum ether:Ethyl acetate=1:0 to 0:1) to give the title compound (198-2) (10.0 g, crude) as a white solid. LCMS (ES⁺): m/z 452.2 [M+H]⁺.

Synthesis of (S)-1-(3-(5-fluoro-2-(pyrrolidin-3-ylamino)pyrimidin-4-yl)phenyl)pyridin-2(1H)-one (198-3). A mixture of tert-butyl (S)-3-((5-fluoro-4-(3-(2-oxopyridin-1 (2H)-yl)phenyl)pyrimidin-2-yl)amino)pyrrolidine-1-carboxylate (198-2) (300 mg, 664 μmol, 1.00 eq) in HCl/EtOAc (4.00 M, 5 mL, 30.1 eq) was stirred at 25 °C for 1 h. LC-MS showed the desired mass was detected. The reaction mixture was concentrated under reduced pressure to give the title compound (198-3) (300 mg, crude, HCl) as a white solid. LCMS (ES⁺): m/z 352.1 [M+H]⁺.

Synthesis of (S)-1-(3-(2-((1-acetylpyrrolidin-3-yl)amino)-5-fluoropyrimidin-4-yl)phenyl)pyridin-2(1H)-one (Compound 613). To a solution of (S)-1-(3-(5-fluoro-2-(pyrrolidin-3-ylamino)pyrimidin-4-yl)phenyl)pyridin-2(1H)-one (198-3) (300 mg, 853 μmol, 1.00 eq, HCl) in DCM (10 mL) was added TEA (259 mg, 2.56 mmol, 356 μL, 3.00 eq) and acetyl chloride (53.6 mg, 683 μmol, 48.5 μL, 0.800 eq) at 25 °C. The mixture was stirred at 25 °C for 1 h. LC-MS showed the desired mass was detected. The reaction mixture was quenched by addition H₂O 5 mL, and then extracted with DCM 3 mL*3. The combined organic layers were dried over Na₂SO₄, filtered and concentrated under reduced pressure. The residue was purified by prep-HPLC (column: Phenomenex Luna C18 100*30 mm*3 um; mobile phase: [H₂O (0.2% FA)-ACN]; gradient: 5%-45% B over 8.0 min) to give the title compound (Compound 613) (85.6 mg, 216 μmol, 25.3% yield, 99.4% purity) as a white solid. LCMS (ES⁺): m/z 394.2 [M+H]⁺.

### Example 199. Synthesis of trans-N-(4-((5-fluoro-4-(2-(1-hydroxycyclobutyl)pyridin-4-yl)pyrimidin-2-yl)amino)cyclohexyl)acetamide (Compound 231)

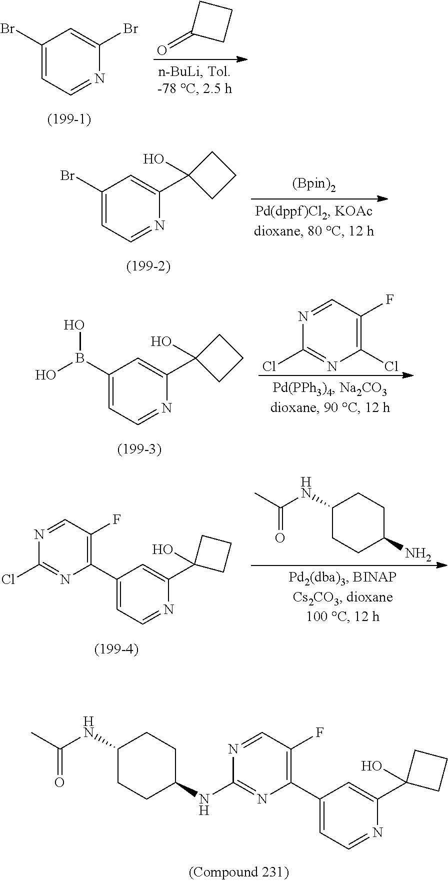

Synthesis of 1-(4-bromopyridin-2-yl)cyclobutan-1-ol (199-2). To a solution of 2,4-dibromopyridine (199-1) (12.0 g, 50.7 mmol, 1.00 eq) in toluene (200 mL) was added n-BuLi (2.50 M, 20.3 mL, 1.00 eq) dropwise at −78 °C under N₂ atmosphere. The mixture was stirred −78 °C for 0.5 h under N₂. Then a solution of cyclobutanone (3.55 g, 50.7 mmol, 3.79 mL, 1.00 eq) in toluene (30 mL) was slowly dropwise to the mixture at −78 °C under N₂. The mixture was stirred −78 °C for 2 h under N₂. LC-MS showed the desired mass was detected. The mixture was warmed to 0 °C, and saturated NH₄Cl aqueous solution (200 mL) was added to the mixture dropwise to quench the reaction at 0 °C under N₂ atmosphere. Then the reaction mixture was extracted with EtOAc (100 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=3:1 to 1:1) to give the title compound (199-2) (5.80 g, 25.4 mmol, 50.2% yield) as a white solid. LCMS (ES⁺): m/z 228.0 [M+H]⁺.

Synthesis of (2-(1-hydroxycyclobutyl)pyridin-4-yl)boronic acid (199-3). To a solution of 1-(4-bromopyridin-2-yl)cyclobutan-1-ol (199-2) (5.00 g, 21.9 mmol, 1.00 eq) in dioxane (100 mL) were added 4,4,5,5-tetramethyl-2-(4,4,5,5-tetramethyl-1,3,2-dioxaborolan-2-yl)-1,3,2-dioxaborolane (6.68 g, 26.3 mmol, 1.20 eq), KOAc (4.30 g, 43.8 mmol, 2.00 eq) and Pd(dppf)Cl₂ (802 mg, 1.10 mmol, 0.0500 eq) at 20 °C under N₂ atmosphere. The mixture was stirred at 80 °C for 12 h under N₂. LC-MS showed 1-(4-bromopyridin-2-yl)cyclobutan-1-ol (199-2) was consumed completely and desired mass was detected. The mixture was used into the next step without further work up and purification to give the title compound (199-3) (4.23 g, crude) in dioxane (100 mL) as a brown liquid. LCMS (ES⁺): m/z 194.1 [M+H]⁺.

Synthesis of 1-(4-(2-chloro-5-fluoropyrimidin-4-yl)pyridin-2-yl)cyclobutan-1-ol (199-4). To a solution of (2-(1-hydroxycyclobutyl)pyridin-4-yl)boronic acid (199-3) (4.23 g, 21.9 mmol, 1.00 eq) in dioxane (100 mL) and H₂O (10 mL) were added 2,4-dichloro-5-fluoro-pyrimidine (3.66 g, 21.9 mmol, 1.00 eq), Pd(PPh₃)₄ (507 mg, 438 μmol, 0.0200 eq) and Na₂CO₃ (4.64 g, 43.8 mmol, 2.00 eq) at 20 °C under N₂ atmosphere. The mixture was stirred at 90 °C for 12 h under N₂. LC-MS showed ~7% of (2-(1-hydroxycyclobutyl)pyridin-4-yl)boronic acid (199-3) was remaining and ~30% of the desired mass was detected. Water (50 mL) was added to the mixture at 20 °C. Then the reaction mixture was extracted with EtOAc (30 mL*3). The combined organic layers were dried over anhydrous sodium sulfate, filtered and the filtrate was concentrated under reduced pressure to give a residue. The residue was purified by column chromatography (SiO₂, Petroleum ether:Ethyl acetate=10 1 to 2:1) to give the title compound (199-4) (2.90 g, 10.4 mmol, 44.5% yield) as a white solid. LCMS (ES⁺): m/z 280.1 [M+H]⁺.

Synthesis of trans-N-(4-((5-fluoro-4-(2-(1-hydroxycyclobutyl)pyridin-4-yl)pyrimidin-2-yl)amino)cyclohexyl)acetamide (Compound 231). To a solution of 1-(4-(2-chloro-5-fluoropyrimidin-4-yl)pyridin-2-yl)cyclobutan-1-ol (199-4) (300 mg, 1.07 mmol, 1.00 eq) in dioxane (7 mL) were added trans-N-(4-aminocyclohexyl)acetamide (335 mg, 2.15 mmol, 2.00 eq), Pd₂(dba)₃ (98.2 mg, 107 μmol, 0.100 eq), Cs₂CO₃ (699 mg, 2.15 mmol, 2.00 eq) and BINAP (66.8 mg, 107 μmol, 0.100 eq) at 20 °C under N₂ atmosphere. The mixture was stirred at 100 °C for 12 h under N₂. LC-MS showed 1-(4-(2-chloro-5-fluoropyrimidin-4-yl)pyridin-2-yl)cyclobutan-1-ol (199-4) was consumed completely and the desired mass was detected. The reaction mixture was concentrated under reduced pressure to remove solvent. The residue was purified by prep-HPLC (column: Waters Xbridge Prep OBD C18 150*40 mm*10 um; mobile phase: [H₂O (10 mM NH₄HCO₃)-ACN]; gradient: 40%-70% B over 8.0 min) to give the title compound (Compound 231) (25.0 mg, 59.0 μmol, 5.50% yield, 94.3% purity) as a white solid. LCMS (ES⁺): m/z 400.2 [M+H]⁺.

### Example 200. Synthesis of trans-4-((5-fluoro-4-(2-(1-hydroxycyclobutyl)pyridin-4-yl)pyrimidin-2-yl)amino)cyclohexane-1-carboxamide (Compound 243)

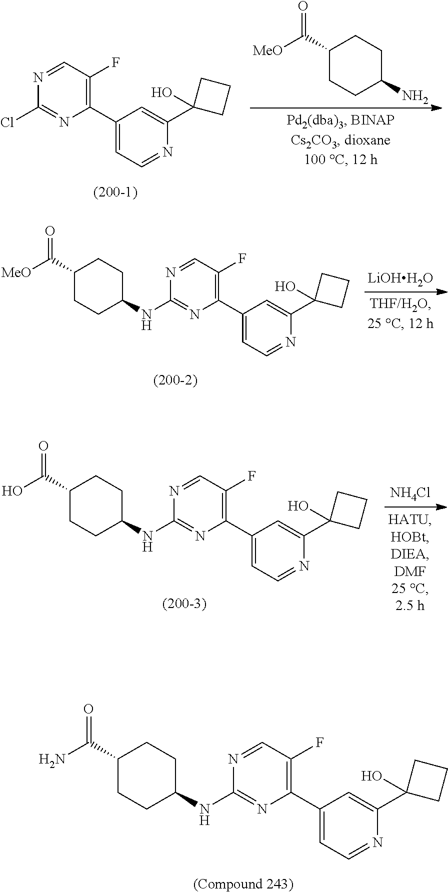

Synthesis of trans-methyl-4-((5-fluoro-4-(2-(I-hydroxycyclobutyl)pyridin-4-yl)pyrimidin-2-yl)amino)cyclohexane-1-carboxylate (200-2). To a solution of 1-(4-(2-chloro-5-fluoropyrimidin-4-yl)pyridin-2-yl)cyclobutan-1-ol (200-1) (1.00 g, 3.58 mmol, 1.00 eq) and trans-methyl 4-aminocyclohexanecarboxylate (618 mg, 3.93 mmol, 1.10 eq) in dioxane (15 mL) were added BINAP (445 mg, 715 μmol, 0.200 eq), Cs₂CO₃ (1.75 g, 5.36 mmol, 1.50 eq) and Pd₂(dba)₃ (655 mg, 715 μmol, 0.200 eq) at 25 °C under N₂ atmosphere. The mixture was stirred at 100 °C for 12 h under N₂. LCMS showed 1-(4-(2-chloro-5-fluoropyrimidin-4-yl)pyridin-2-yl)cyclobutan-1-ol (200-1) was consumed completely and the desired mass was detected. The reaction mixture filtered and the filtrate was concentrated under reduced pressure to give a residue. The residue was purified by column chromatography (SiO₂, Petroleum ether:Ethyl acetate=3:1 to 1:1) to give the title compound (200-2) (651 mg, 1.26 mmol, 35.2% yield, 77.4% purity) as a yellow oil. LCMS (ES⁺): m/z 401.2 [M+H]⁺.

Synthesis of trans-4-((5-fluoro-4-(2-(1-hydroxycyclobutyl)pyridin-4-yl)pyrimidin-2-yl)amino)cyclohexane-1-carboxylic acid (200-3). To a solution of trans-methyl-4-((5-fluoro-4-(2-(1-hydroxycyclobutyl)pyridin-4-yl)pyrimidin-2-yl)amino)cyclohexane-1-carboxylate (200-2) (600 mg, 1.50 mmol, 1.00 eq) in THF (5 mL) and H₂O (2.5 mL) was added LiOH·H₂O (252 mg, 5.99 mmol, 4.00 eq) at 25 °C. The mixture was stirred at 25 °C for 12 h. LCMS showed trans-methyl-4-((5-fluoro-4-(2-(1-hydroxycyclobutyl)pyridin-4-yl)pyrimidin-2-yl)amino)cyclohexane-1-carboxylate (200-2) was consumed completely and the desired mass was detected. The reaction was concentrated under reduced pressure to remove THF. Then to the remaining aqueous phase was added HCl (1 M) dropwise to adjust pH to 3. Solid was precipitated in the mixture. Then it was filtered and the filter cake was washed with H₂O (10 mL), dried in vacuum to give the title compound (200-3) (520 mg, 1.21 mmol, 80.4% yield, 89.6% purity) as a white solid. LCMS (ES⁺): m/z 387.3 [M+H]⁺.

Synthesis of trans-4-((5-fluoro-4-(2-(1-hydroxycyclobutyl)pyridin-4-yl)pyrimidin-2-yl)amino)cyclohexane-1-carboxamide (Compound 243). To a solution of trans-4-((5-fluoro-4-(2-(1-hydroxycyclobutyl)pyridin-4-yl)pyrimidin-2-yl)amino)cyclohexane-1-carboxylic acid (200-3) (300 mg, 776 μmol, 1.00 eq) and NH₄Cl (166 mg, 3.11 mmol, 4.00 eq) in DMF (3 mL) were added DIEA (502 mg, 3.88 mmol, 676 μL, 5.00 eq), HATU (354 mg, 932 μmol, 1.20 eq) and HOBt (52.5 mg, 388 μmol, 0.500 eq) at 25 °C. The mixture was stirred at 25 °C for 2.5 h. LCMS showed trans-4-((5-fluoro-4-(2-(1-hydroxycyclobutyl)pyridin-4-yl)pyrimidin-2-yl)amino)cyclohexane-1-carboxylic acid (200-3) was consumed completely and the desired mass was detected. The mixture was filtered and the filtrate was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 10%-40% B over 8.0 min) to give the title compound (Compound 243) (114 mg, 295 μmol, 38.0% yield, 99.6% purity) as a white solid. LCMS (ES⁺): m/z 386.2 [M+H]⁺.

### Example 201. Synthesis of 1-(4-((5-fluoro-4-(2-(1-hydroxycyclobutyl)pyridin-4-yl)pyrimidin-2-yl)amino)piperidin-1-yl)ethan-1-one (Compound 232)

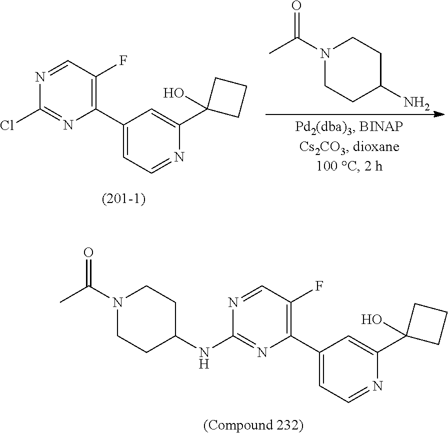

Synthesis of 1-(4-((5-fluoro-4-(2-(1-hydroxycyclobutyl)pyridin-4-yl)pyrimidin-2-yl)amino)piperidin-1-yl)ethan-1-one (Compound 232). To a solution of 1-(4-(2-chloro-5-fluoropyrimidin-4-yl)pyridin-2-yl)cyclobutan-1-ol (201-1) (300 mg, 1.07 mmol, 1.00 eq) in dioxane (7 mL) were added 1-(4-amino-1-piperidyl)ethanone (198 mg, 1.39 mmol, 1.30 eq), Cs₂CO₃ (699 mg, 2.15 mmol, 2.00 eq), Pd₂(dba)₃ (98.2 mg, 107 μmol, 0.100 eq) and BINAP (66.8 mg, 107 μmol, 0.100 eq) at 20 °C under N₂ atmosphere. The mixture was stirred at 100 °C for 2 h under N₂. LC-MS showed 1-(4-(2-chloro-5-fluoropyrimidin-4-yl)pyridin-2-yl)cyclobutan-1-ol (201-1) was consumed completely and the desired mass was detected. The reaction mixture was concentrated under reduced pressure to remove solvent. The residue was purified by prep-HPLC (column: Phenomenex Gemini-NX 80*40 mm*3 um; mobile phase: [H₂O (10 mM NH₄HCO₃)-ACN]; gradient: 25%-55% B over 8.0 min) to give the title compound (Compound 232) (88.3 mg, 228 μmol, 21.3% yield, 99.6% purity) as a white solid. LCMS (ES⁺): m/z 386.2 [M+H]⁺.

### Example 202. Synthesis of (1R,3S)-3-((5-fluoro-4-(2-(1-hydroxycyclobutyl)pyridin-4-yl)pyrimidin-2-yl)amino)cyclohexane-1-carboxamide (Compound 244)

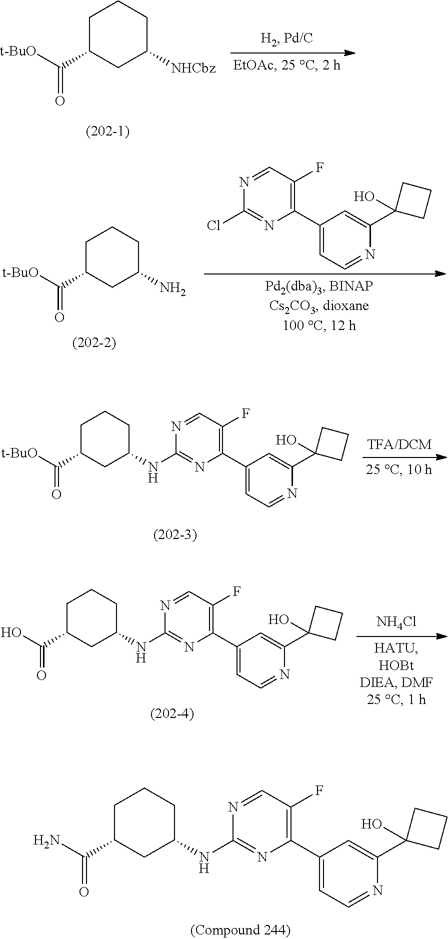

Synthesis of tert-butyl (1R,3S)-3-aminocyclohexane-1-carboxylate (202-2). 10% Pd/C (4.00 g, 50% purity) was added to EtOAc (100 mL) at 25 °C under N₂ atmosphere, then tert-butyl (1R,3S)-3-(((benzyloxy)carbonyl)amino)cyclohexane-1-carboxylate (202-1) (2.00 g, 6.00 mmol, 1.00 eq) was added to the mixture at 25 °C, the suspension was degassed and purged with H₂ for three times. The mixture was stirred at 25 °C for 2 h under H₂ (15 psi) atmosphere. LC-MS showed rac-tert-butyl (1R,3S)-3-(((benzyloxy)carbonyl)amino)cyclohexane-1-carboxylate (202-1) was consumed completely and the desired mass was detected. The reaction mixture was filtered and the filtrate was concentrated under reduced pressure to give the title compound (202-2) (1.63 g, crude) as a light yellow oil. LCMS (ES⁺): m/z 200.2 [M+H]⁺.

Synthesis of tert-butyl (1R,3S)-3-((5-fluoro-4-(2-(1-hydroxycyclobutyl)pyridin-4-yl)pyrimidin-2-yl)amino)cyclohexane-J-carboxylate (202-3). To a solution of 1-(4-(2-chloro-5-fluoropyrimidin-4-yl)pyridin-2-yl)cyclobutan-1-ol (0.920 g, 3.29 mmol, 1.00 eq) in dioxane (15 mL) were added tert-butyl (1R,3S)-3-aminocyclohexane-1-carboxylate (202-2) (656 mg, 3.29 mmol, 1.00 eq), Cs₂CO₃ (3.22 g, 9.87 mmol, 3.00 eq), BINAP (205 mg, 329 μmol, 0.100 eq) and Pd₂(dba)₃ (301 mg, 329 μmol, 0.100 eq) at 25 °C under N₂ atmosphere. The mixture was stirred at 100 °C for 12 h under N₂. LC-MS showed tert-butyl (1R,3S)-3-aminocyclohexane-1-carboxylate (202-2) was consumed completely and the desired mass was detected. Water (15 mL) was added to the mixture at 25 °C. The reaction mixture was extracted with EtOAc (10 mL*5). The combined organic layers were dried over anhydrous sodium sulfate, filtered and the filtrate was concentrated under reduced to give a residue. The residue was purified by column chromatography (SiO₂, Petroleum ether:Ethyl acetate=3:1 to 2:1) to give the title compound (202-3) (1.40 g, 3.16 mmol, 96.2% yield) as a yellow oil. LCMS (ES⁺): m/z 443.3 [M+H]⁺.

Synthesis of (1R,3S)-3-((5-fluoro-4-(2-(1-hydroxycyclobutyl)pyridin-4-yl)pyrimidin-2-yl)amino)cyclohexane-1-carboxylic acid (202-4). To a solution of tert-butyl (1R,3S)-3-((5-fluoro-4-(2-(1-hydroxycyclobutyl)pyridin-4-yl)pyrimidin-2-yl)amino)cyclohexane-1-carboxylate (202-3) (400 mg, 904 μmol, 1.00 eq) in DCM (3 mL) was added TFA (1.54 g, 13.5 mmol, 1.00 mL, 14.9 eq) at 25 °C. The mixture was stirred at 25 °C for 10 h. LC-MS showed tert-butyl (1R,3S)-3-((5-fluoro-4-(2-(1-hydroxycyclobutyl)pyridin-4-yl)pyrimidin-2-yl)amino)cyclohexane-1-carboxylate (202-3) was consumed completely and the desired mass was detected. The reaction mixture was concentrated under reduced pressure to give the title compound (202-4) (452 mg, crude, TFA) as a yellow oil. LCMS (ES⁺): m/z 387.1 [M+H]⁺.

Synthesis of (1R,3S)-3-((5-fluoro-4-(2-(I-hydroxycyclobutyl)pyridin-4-yl)pyrimidin-2-yl)amino)cyclohexane-J-carboxamide (Compound 244). To a solution of (1R,3S)-3-((5-fluoro-4-(2-(1-hydroxycyclobutyl)pyridin-4-yl)pyrimidin-2-yl)amino)cyclohexane-1-carboxylic acid (202-4) (450 mg, 899 μmol, 1.00 eq, TFA) in DMF (3 mL) were added DIEA (581 mg, 4.50 mmol, 783 μL, 5.00 eq), HATU (410 mg, 1.08 mmol, 1.20 eq), HOBt (60.8 mg, 450 μmol, 0.500 eq) and NH₄Cl (192 mg, 3.60 mmol, 4.00 eq) at 25 °C. The mixture was stirred at 25 °C for 1 h. LC-MS showed (1R,3S)-3-((5-fluoro-4-(2-(1-hydroxycyclobutyl)pyridin-4-yl)pyrimidin-2-yl)amino)cyclohexane-1-carboxylic acid (202-4) was consumed completely and the desired mass was detected. The reaction mixture was concentrated under reduced pressure to remove solvent to give a residue. The residue was purified by prep-HPLC (column: Waters Xbridge BEH C18 100*30 mm*10 um; mobile phase: [1H₂O (10 mM NH₄HCO₃)-ACN]; gradient: 25%-55% B over 8.0 min) to give the title compound (Compound 244) (65.2 mg, 169 μmol, 18.8% yield, 100% purity) as a yellow solid. LCMS (ES⁺): m/z 386.1 [M+H]⁺.

### Example 203. Synthesis of trans-N-(4-((5-fluoro-4-(2-(2-oxopyrrolidin-1-yl)pyridin-4-yl)pyrimidin-2-yl)amino)cyclohexyl)acetamide (Compound 261)

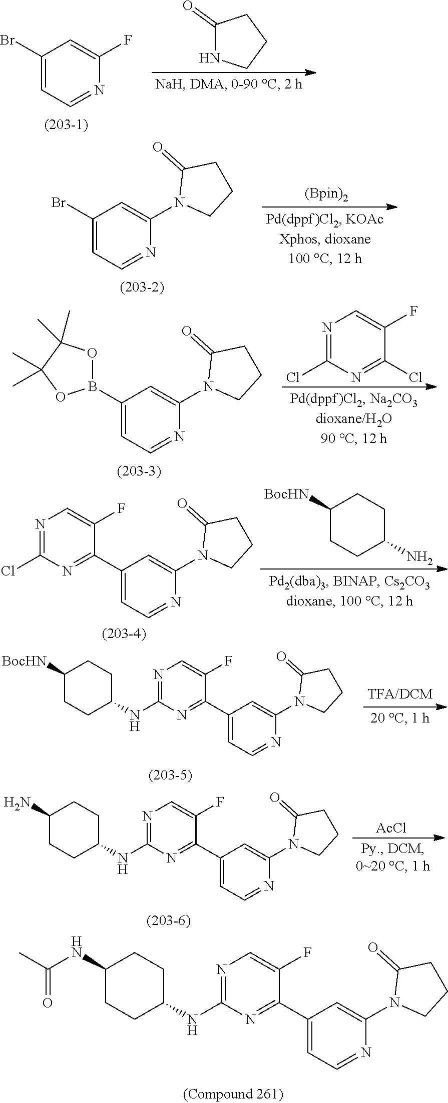

Synthesis of 1-(4-bromopyridin-2-yl)pyrrolidin-2-one (203-2). To a solution of 4-bromo-2-fluoropyridine (203-1) (12.0 g, 68.2 mmol, 1.00 eq) and pyrrolidin-2-one (5.80 g, 68.2 mmol, 5.23 mL, 1.00 eq) in DMA (120 mL) was added NaH (2.86 g, 71.6 mmol, 60% purity, 1.05 eq) at 0 °C under N₂. The mixture was stirred at 90 °C for 2 h under N₂. LCMS showed that 4-bromo-2-fluoropyridine (203-1) was consumed completely and one main peak with desired MS was detected. The reaction mixture was poured into saturated NH₄Cl solution (200 mL) and filtered to give a filter cake. The filter cake was washed with H₂O (30 mL) and dried under reduced pressure. The crude product was triturated with Petroleum ether (100 mL) at 20 °C for 30 min to give title compound (203-2) (9.50 g, 39.4 mmol, 57.8% yield) as a yellow solid. LCMS (ES⁺): m/z 241.0 [M+H]⁺.

Synthesis of 1-(4-(4,4,5,5-tetramethyl-1,3,2-dioxaborolan-2-yl)pyridin-2-yl)pyrrolidin-2-one (203-3). To a solution of 1-(4-bromopyridin-2-yl)pyrrolidin-2-one (203-2) (9.50 g, 39.4 mmol, 1.00 eq) and 4,4,5,5-tetramethyl-2-(4,4,5,5-tetramethyl-1,3,2-dioxaborolan-2-yl)-1,3,2-dioxaborolane (12.0 g, 47.3 mmol, 1.20 eq) in dioxane (100 mL) were added Pd(dppf)Cl₂ (2.88 g, 3.94 mmol, 0.100 eq), KOAc (7.73 g, 78.8 mmol, 2.00 eq) and XPhos (3.76 g, 7.88 mmol, 0.200 eq) at 20 °C. The mixture was stirred at 100 °C for 12 h under N₂. LCMS showed that 1-(4-bromopyridin-2-yl)pyrrolidin-2-one (203-2) was consumed completely and one main peak with desired MS was detected. The reaction mixture was filtered and concentrated under reduced pressure to give title compound (203-3) (11.5 g, crude) as a brown solid. LCMS (ES⁺): m/z 289.0 [M+H]⁺.

Synthesis of 1-(4-(2-chloro-5-fluoropyrimidin-4-yl)pyridin-2-yl)pyrrolidin-2-one (203-4). To a solution of 1-(4-(4,4,5,5-tetramethyl-1,3,2-dioxaborolan-2-yl)pyridin-2-yl)pyrrolidin-2-one (203-3) (11.5 g, 39.9 mmol, 1.00 eq) and 2,4-dichloro-5-fluoropyrimidine (6.66 g, 39.9 mmol, 1.00 eq) in dioxane (100 mL) and H₂O (10 mL) were added Pd(dppf)Cl₂ (2.92 g, 3.99 mmol, 0.100 eq) and Na₂CO₃ (8.46 g, 79.8 mmol, 2.00 eq) at 20 °C. The mixture was stirred at 90 °C for 12 h under N₂. LCMS showed that 1-(4-(4,4,5,5-tetramethyl-1,3,2-dioxaborolan-2-yl)pyridin-2-yl)pyrrolidin-2-one (203-3) was consumed completely and one main peak with desired MS was detected. The reaction mixture was diluted with H₂O (200 mL) and extracted with EtOAc (150 mL*3). The combined organic layers were washed with brine (100 mL), dried over anhydrous Na₂SO₄, filtered and concentrated under reduced pressure. The residue was purified by column chromatography (SiO₂, Petroleum ether/Ethyl acetate=100/1 to 80/20) to give title compound (203-4) (5.70 g, 19.5 mmol, 48.8% yield) as a yellow solid. LCMS (ES⁺): m/z 293.0 [M+H]⁺.

Synthesis of trans-tert-butyl (4-((5-fluoro-4-(2-(2-oxopyrrolidin-1-yl)pyridin-4-yl)pyrimidin-2-yl)amino)cyclohexyl)carbamate (203-5). To a solution of 1-(4-(2-chloro-5-fluoropyrimidin-4-yl)pyridin-2-yl)pyrrolidin-2-one (203-4) (1.00 g, 3.42 mmol, 1.00 eq) and trans-tert-butyl (4-aminocyclohexyl)carbamate (732 mg, 3.42 mmol, 1.00 eq) in dioxane (15 mL) were added Pd₂(dba)₃ (313 mg, 342 μmol, 0.100 eq), Cs₂CO₃ (2.23 g, 6.83 mmol, 2.00 eq) and BINAP (425 mg, 683 μmol, 0.200 eq) at 20 °C. The mixture was stirred at 100 °C for 12 h under N₂. LCMS showed that 1-(4-(2-chloro-5-fluoropyrimidin-4-yl)pyridin-2-yl)pyrrolidin-2-one (203-4) was consumed completely and one main peak with desired MS was detected. The reaction mixture was concentrated under reduced pressure to remove solvent. The residue was diluted with H₂O (30 mL) and extracted with EtOAc (20 mL*3). The combined organic layers were washed with brine (30 mL), dried over anhydrous Na₂SO₄, filtered and concentrated under reduced pressure. The residue was purified by column chromatography (SiO₂, Petroleum ether/Ethyl acetate=100/1 to 80/20) to give title compound (203-5) (640 mg, 1.36 mmol, 39.8% yield) as a white solid. LCMS (ES⁺): m/z 471.0 [M+H]⁺.

Synthesis of trans-1-(4-(2-((4-aminocyclohexyl)amino)-5-fluoropyrimidin-4-yl)pyridin-2-yl)pyrrolidin-2-one (203-6). To a solution of trans-tert-butyl (4-((5-fluoro-4-(2-(2-oxopyrrolidin-1-yl)pyridin-4-yl)pyrimidin-2-yl)amino)cyclohexyl)carbamate (203-5) (200 mg, 425 μmol, 1.00 eq) in DCM (2 mL) was added TFA (768 mg, 6.73 mmol, 0.5 mL) at 20 °C. The mixture was stirred at 20 °C for 1 h. LCMS showed that trans-tert-butyl (4-((5-fluoro-4-(2-(2-oxopyrrolidin-1-yl)pyridin-4-yl)pyrimidin-2-yl)amino)cyclohexyl)carbamate (203-5) was consumed completely and one main peak with desired MS was detected. The reaction mixture was concentrated under reduced pressure to remove solvent to give title compound (203-6) (200 mg, crude, TFA) as a yellow oil. LCMS (ES⁺): m/z 371.0 [M+H]⁺.

Synthesis of trans-N-(4-((5-fluoro-4-(2-(2-oxopyrrolidin-1-yl)pyridin-4-yl)pyrimidin-2-yl)amino)cyclohexyl)acetamide (Compound 261). To a solution of trans-1-(4-(2-((4-aminocyclohexyl)amino)-5-fluoropyrimidin-4-yl)pyridin-2-yl)pyrrolidin-2-one (203-6) (200 mg, 413 μmol, 1.00 eq, TFA) in DCM (4 mL) was added Py (98.0 mg, 1.24 mmol, 100 μL, 3.00 eq) and acetyl chloride (32.4 mg, 413 μmol, 29.4 μL, 1.00 eq) at 0 °C. The mixture was stirred at 20 °C for 1 h under N₂. LCMS showed that trans-1-(4-(2-((4-aminocyclohexyl)amino)-5-fluoropyrimidin-4-yl)pyridin-2-yl)pyrrolidin-2-one (203-6) was consumed completely and one main peak with desired MS was detected. The reaction mixture was concentrated under reduced pressure to remove solvent. The residue was washed with H₂O (10 mL) and filtered to give a filter cake as a crude product. The residue was purified by prep-HPLC (column: Phenomenex luna C18 100*40 mm*3 um; mobile phase: [H₂O (0.2% FA)-ACN]; gradient: 15%-45% B over 8.0 min) to give title compound (Compound 261) (41.9 mg, 92.31 μmol, 22.36% yield, 100% purity) as a white solid. LCMS (ES⁺): m/z 413.2 [M+H]⁺.

### Example 204. Synthesis of trans-(1r,4r)-4-((5-fluoro-4-(2-(2-oxopyrrolidin-1-yl)pyridin-4-yl)pyrimidin-2-yl)amino)cyclohexane-1-carboxamide (Compound 262)

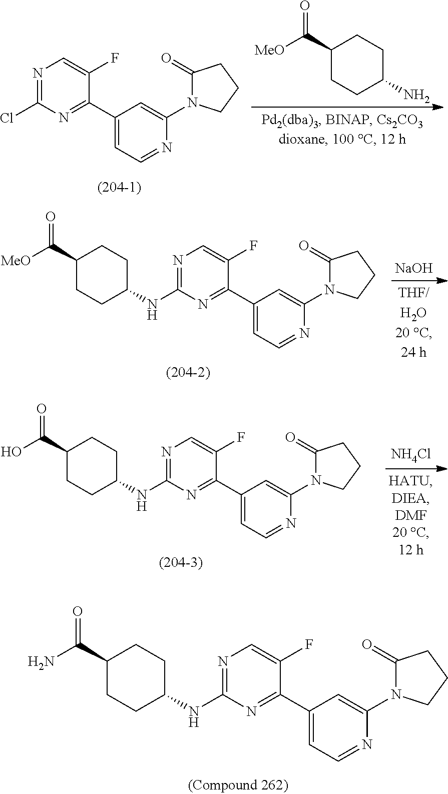

Synthesis of trans-methyl (1r,4r)-4-((5-fluoro-4-(2-(2-oxopyrrolidin-1-yl)pyridin-4-yl)pyrimidin-2-yl)amino)cyclohexane-1-carboxylate (204-2). To a solution of 1-(4-(2-chloro-5-fluoropyrimidin-4-yl)pyridin-2-yl)pyrrolidin-2-one (204-1) (1.00 g, 3.42 mmol, 1.00 eq) in dioxane (20 mL) were added trans-methyl (1r,4r)-4-aminocyclohexane-1-carboxylate (591 mg, 3.76 mmol, 1.10 eq), Cs₂CO₃ (2.23 g, 6.83 mmol, 2.00 eq), BINAP (213 mg, 342 μmol, 0.100 eq) and Pd₂(dba)₃ (313 mg, 342 μmol, 0.100 eq) at 20 °C. The mixture was stirred at 100 °C for 12 h under N₂. LC-MS showed that 1-(4-(2-chloro-5-fluoropyrimidin-4-yl)pyridin-2-yl)pyrrolidin-2-one (204-1) was consumed completely and one main peak with the desired mass was detected. The mixture was filtered and the filtrate was concentrated under reduced pressure. The residue was purified by column chromatography (SiO₂, Petroleum ether:Ethyl acetate=1:0 to 1:1) to give the title compound (204-2) (800 mg, 1.93 mmol, 56.6% yield, n/a purity) as a yellow solid. LCMS (ES⁺): m/z 414.2 [M+H]⁺.

Synthesis of trans-(1r,4r)-4-((5-fluoro-4-(2-(2-oxopyrrolidin-1-yl)pyridin-4-yl)pyrimidin-2-yl)amino)cyclohexane-1-carboxylic acid (204-3). To a solution of trans-methyl (1r,4r)-4-((5-fluoro-4-(2-(2-oxopyrrolidin-1-yl)pyridin-4-yl)pyrimidin-2-yl)amino)cyclohexane-1-carboxylate (204-2) (800 mg, 1.93 mmol, 1.00 eq) in THF (4 mL), H₂O (2 mL) was added NaOH (155 mg, 3.87 mmol, 2.00 eq) at 20 °C. The mixture was stirred at 20 °C for 12 h. LC-MS showed trans-methyl (1r,4r)-4-((5-fluoro-4-(2-(2-oxopyrrolidin-1-yl)pyridin-4-yl)pyrimidin-2-yl)amino)cyclohexane-1-carboxylate (204-2) was not consumed completely and one main peak with the desired mass was detected. The mixture was stirred at 20 °C for 12 h. LC-MS showed trans-methyl (1r,4r)-4-((5-fluoro-4-(2-(2-oxopyrrolidin-1-yl)pyridin-4-yl)pyrimidin-2-yl)amino)cyclohexane-1-carboxylate (204-2) was not consumed completely and one main peak with the desired mass was detected. The mixture was concentrated under reduced pressure. The reaction mixture was diluted with H₂O (20 mL) and extracted with EtOAc (20 mL*5). The combined water layers were adjusted pH to 5, and filtered and the filter cake was dried in a vacuum to give the title compound (204-3) (300 mg, crude) as a yellow solid. LCMS (ES⁺): m/z 400.2 [M+H]⁺.

Synthesis of Synthesis of trans-(1r,4r)-4-((5-fluoro-4-(2-(2-oxopyrrolidin-1-yl)pyridin-4-yl)pyrimidin-2-yl)amino)cyclohexane-1-carboxamide (Compound 262). To a solution of trans-(1r,4r)-4-((5-fluoro-4-(2-(2-oxopyrrolidin-1-yl)pyridin-4-yl)pyrimidin-2-yl)amino)cyclohexane-1-carboxylic acid (204-3) (300 mg, 751 μmol, 1.00 eq) in DMF (5 mL) were added NH₄Cl (121 mg, 2.25 mmol, 3.00 eq), DIEA (485 mg, 3.76 mmol, 654 μL, 5.00 eq) and HATU (857 mg, 2.25 mmol, 3.00 eq) at 20 °C. The mixture was stirred at 20 °C for 12 h. LC-MS showed trans-(1r,4r)-4-((5-fluoro-4-(2-(2-oxopyrrolidin-1-yl)pyridin-4-yl)pyrimidin-2-yl)amino)cyclohexane-1-carboxylic acid (204-3) was not consumed completely and one main peak with the desired mass was detected. The mixture was filtered and the filter cake was dried in a vacuum. The residue was purified by prep-HPLC (column: Phenomenex luna C18 100*40 mm*5 um; mobile phase: [H₂O (0.2% FA)-ACN]; gradient: 15%-45% B over 8.0 min) to give the title compound (Compound 262) (101.6 mg, 253 μmol, 33.7% yield, 99.2% purity) as a white solid. LCMS (ES⁺): m/z 399.1 [M+H]⁺.

### Example 205. Synthesis of trans-N-((1r,4r)-4-((5-fluoro-4-(2-(2-oxooxazolidin-3-yl)pyridin-4-yl)pyrimidin-2-yl)amino)cyclohexyl)acetamide (Compound 253)

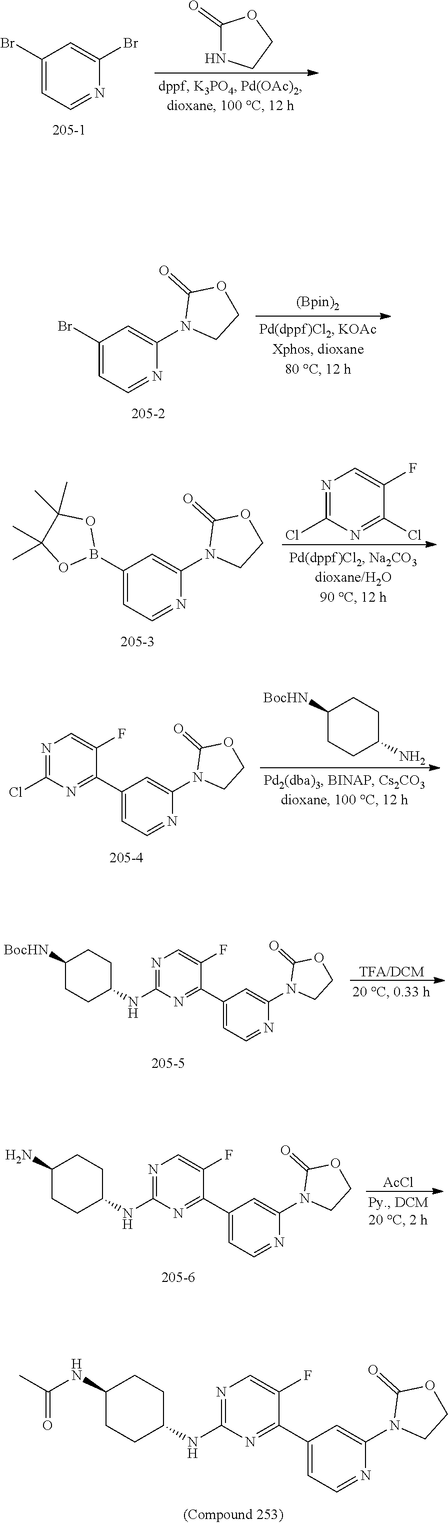

Synthesis of 3-(4-bromopyridin-2-yl)oxazolidin-2-one (205-2). To a solution of 2,4-dibromopyridine (205-1) (1.00 g, 4.22 mmol, 1.00 eq) and oxazolidin-2-one (441 mg, 5.07 mmol, 1.20 eq) in dioxane (10.0 mL) was added Pd(OAc)₂ (94.8 mg, 422 μmol, 0.100 eq), K₃PO₄ (2.24 g, 10.6 mmol, 2.50 eq) and DPPF (468 mg, 844 μmol, 0.200 eq) at 20 °C. The mixture was stirred at 100 °C for 12 h under N₂. LC-MS showed that 2,4-dibromopyridine (205-1) was consumed completely and desired mass was detected. The reaction mixture was concentrated under reduced pressure to remove solvent. The residue was purified by column chromatography (SiO₂, Petroleum ether/Ethyl acetate=1/0 to 5/1) to give the title compound (205-2) (500 mg, crude) as a white solid. LCMS (ES⁺): m/z 242.9 [M+H]⁺

Synthesis of 3-(4-(4,4,5,5-tetramethyl-1,3,2-dioxaborolan-2-yl)pyridin-2-yl)oxazolidin-2-one (205-3). To a solution of 3-(4-bromopyridin-2-yl)oxazolidin-2-one (205-2) (500 mg, 2.06 mmol, 1.00 eq) and 4,4,5,5-tetramethyl-2-(4,4,5,5-tetramethyl-1,3,2-dioxaborolan-2-yl)-1,3,2-dioxaborolane (627 mg, 2.47 mmol, 1.20 eq) in dioxane (10 mL) was added Pd(dppf)Cl₂ (151 mg, 206 μmol, 0.1 eq), KOAc (404 mg, 4.11 mmol, 2.00 eq) and XPhos (196 mg, 411 μmol, 0.2 eq) at 20 °C. The mixture was stirred at 80 °C for 12 h under N₂. LC-MS showed that 3-(4-bromopyridin-2-yl)oxazolidin-2-one (205-2) was consumed completely and desired mass was detected. The mixture was filtered and filtrate was concentrated under reduced pressure to remove solvent to give the title compound (205-3) (550 mg, crude) as a brown solid. LCMS (ES⁺): m/z 291.1 [M+H]⁺

Synthesis of 3-(4-(2-chloro-5-fluoropyrimidin-4-yl)pyridin-2-yl)oxazolidin-2-one (205-4). To a solution of 3-(4-(4,4,5,5-tetramethyl-1,3,2-dioxaborolan-2-yl)pyridin-2-yl)oxazolidin-2-one (205-3) (500 mg, 1.72 mmol, 1 eq) and 2,4-dichloro-5-fluoropyrimidine (345 mg, 2.07 mmol, 1.2 eq) in dioxane (2 mL) was added Pd(dppf)Cl₂ (126 mg, 172 μmol, 0.1 eq), H₂O (0.2 mL) and Na₂CO₃ (365 mg, 3.45 mmol, 2.00 eq) at 20 °C. The mixture was stirred at 90 °C for 12 h under N₂. LC-MS showed 3-(4-(4,4,5,5-tetramethyl-1,3,2-dioxaborolan-2-yl)pyridin-2-yl)oxazolidin-2-one (205-3) was consumed completely and desired mass was detected. The mixture was filtered and filtrate was concentrated under reduced pressure to remove solvent. The residue was purified by column chromatography (SiO₂, Petroleum ether/Ethyl acetate=1/0 to 1/3) to give the title compound (205-4) (430 mg, crude) as a white solid. LCMS (ES⁺): m/z 295.0 [M+H]⁺

Synthesis of trans-tert-butyl ((1r,4r)-4-((5-fluoro-4-(2-(2-oxooxazolidin-3-yl)pyridin-4-yl)pyrimidin-2-yl)amino)cyclohexyl)carbamate (205-5). To a solution of 3-(4-(2-chloro-5-fluoropyrimidin-4-yl)pyridin-2-yl)oxazolidin-2-one (205-4) (400 mg, 1.36 mmol, 1 eq) and trans-tert-butyl ((1r,4r)-4-aminocyclohexyl)carbamate (291 mg, 1.36 mmol, 1 eq) in dioxane (10 mL) was added Pd₂(dba)₃ (124 mg, 136 μmol, 0.1 eq), Cs₂CO₃ (1.11 g, 3.39 mmol, 2.50 eq) and BINAP (169 mg, 271 μmol, 0.2 eq) at 20 °C. The mixture was stirred at 100 °C for 12 h under N₂. LC-MS showed that 3-(4-(2-chloro-5-fluoropyrimidin-4-yl)pyridin-2-yl)oxazolidin-2-one (205-4) was consumed completely and desired mass was detected. The mixture was filtered and filtrate was concentrated under reduced pressure to remove solvent. The residue was purified by column chromatography (SiO₂, Petroleum ether/Ethyl acetate=1/0 to 3/1) to give the title compound (205-5) (400 mg, crude) as a white solid. LCMS (ES⁺): m/z 473.2 [M+H]⁺

Synthesis of trans-3-(4-(2-(((1r,4r)-4-aminocyclohexyl)amino)-5-fluoropyrimidin-4-yl)pyridin-2-yl)oxazolidin-2-one (205-6). To a solution of trans-tert-butyl ((1r,4r)-4-((5-fluoro-4-(2-(2-oxooxazolidin-3-yl)pyridin-4-yl)pyrimidin-2-yl)amino)cyclohexyl)carbamate (205-5) (170 mg, 360 μmol, 1 eq) in DCM (3 mL) was added TFA (1.57 g, 13.7 mmol, 1.02 mL, 38.2 eq) at 20 °C. The mixture was stirred at 20 °C for 20 min. LC-MS showed that trans-tert-butyl ((1r,4r)-4-((5-fluoro-4-(2-(2-oxooxazolidin-3-yl)pyridin-4-yl)pyrimidin-2-yl)amino)cyclohexyl)carbamate (205-5) was consumed completely and desired mass was detected. The reaction mixture was concentrated under reduced pressure to remove solvent to give the title compound (205-6) (130 mg, crude, TFA) was obtained as a yellow solid. LCMS (ES⁺): m/z 373.1 [M+H]⁺

Synthesis of trans-N-((1r,4r)-4-((5-fluoro-4-(2-(2-oxooxazolidin-3-yl)pyridin-4-yl)pyrimidin-2-yl)amino)cyclohexyl)acetamide (Compound 253). To a solution of trans-3-(4-(2-(((1r,4r)-4-aminocyclohexyl)amino)-5-fluoropyrimidin-4-yl)pyridin-2-yl)oxazolidin-2-one (205-6) (130 mg, 267 μmol, 1 eq, TFA) and acetyl chloride (16.8 mg, 214 μmol, 15.2 μL, 0.800 eq) in DCM (3 mL) was added Py (42.3 mg, 535 μmol, 43.1 μL, 2.00 eq) at 0 °C. The mixture was stirred at 20 °C for 2 h. LC-MS showed that trans-3-(4-(2-(((1r,4r)-4-aminocyclohexyl)amino)-5-fluoropyrimidin-4-yl)pyridin-2-yl)oxazolidin-2-one (205-6) was consumed completely and desired mass was detected. The mixture washed with THF (5 ml), filtered and concentrated under reduced pressure to give a residue, the filtrate cake was dried over in vacuo to give the crude product, The crude product was purified by re-crystallization from THE (5 mL) at 20 °C to give the title compound (Compound 253) (82.3 mg, 194 μmol, 72.6% yield, 97.7% purity) as a white solid. LCMS (ES⁺): m/z 415.1 [M+H]⁺.

### Example 206. Synthesis of trans-(1r,4r)-4-((5-fluoro-4-(2-(2-oxooxazolidin-3-yl)pyridin-4-yl)pyrimidin-2-yl)amino)cyclohexane-1-carboxamide (Compound 263)

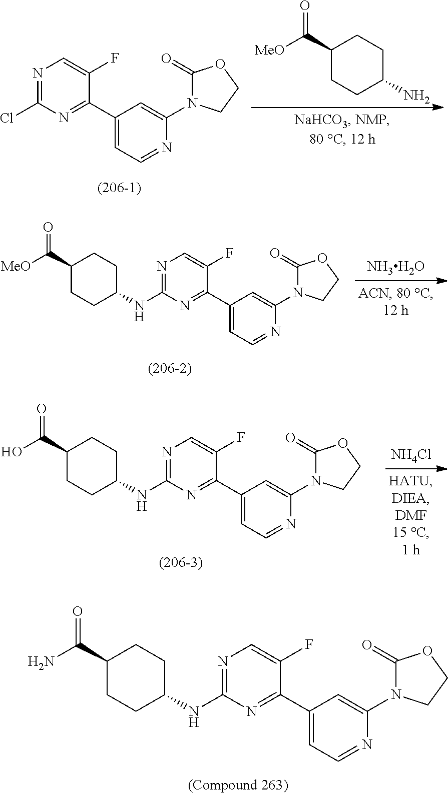

Synthesis of trans-methyl (1r,4r)-4-((5-fluoro-4-(2-(2-oxooxazolidin-3-yl)pyridin-4-yl)pyrimidin-2-yl)amino)cyclohexane-1-carboxylate (206-2). To a solution of 3-(4-(2-chloro-5-fluoropyrimidin-4-yl)pyridin-2-yl)oxazolidin-2-one (206-1) (1.00 g, 3.39 mmol, 1.00 eq) in NMP (20 mL) were added NaHCO₃ (1.14 g, 13.6 mmol, 528 μL, 4.00 eq) and trans-methyl (1r,4r)-4-aminocyclohexane-1-carboxylate (1.31 g, 6.79 mmol, 2.00 eq, HCl) at 15 °C. The mixture was stirred at 80 °C for 12 h. LC-MS showed 3-(4-(2-chloro-5-fluoropyrimidin-4-yl)pyridin-2-yl)oxazolidin-2-one (206-1) was consumed completely, and the desired mass was detected. The mixture was diluted with water (20 mL) and extracted with EtOAc (20 mL*3). The combined organic layers were washed with brine (20 mL*3), dried over Na₂SO₄, filtered and the filtrate was concentrated under reduced pressure. The residue was purified by column chromatography (SiO₂, Petroleum ether/Ethyl acetate=5/1 to 1/1) to give title compound (206-2) (1.00 g, 2.41 mmol, 70.9% yield) as a yellow solid. LCMS (ES⁺): m/z 416.2 [M+H]⁺.

Synthesis of trans-4-((5-fluoro-4-(2-(2-oxooxazolidin-3-yl)pyridin-4-yl)pyrimidin-2-yl)amino)cyclohexane-1-carboxylic acid (206-3). To a solution of trans-methyl 4-((5-fluoro-4-(2-(2-oxooxazolidin-3-yl)pyridin-4-yl)pyrimidin-2-yl)amino)cyclohexane-1-carboxylate (206-2) (880 mg, 2.12 mmol, 1.00 eq) in ACN (3 mL) was added NH₃·H₂O (10.9 g, 77.9 mmol, 12.00 mL, 25% purity, 36.8 eq) at 15 °C. The mixture was stirred at 80 °C for 12 h. LC-MS showed that trans-methyl 4-((5-fluoro-4-(2-(2-oxooxazolidin-3-yl)pyridin-4-yl)pyrimidin-2-yl)amino)cyclohexane-1-carboxylate (206-2) was consumed completely and the desired mass was detected. The mixture was concentrated under reduced pressure. The residue was purified by prep-HPLC (column: Phenomenex luna C18 100*40 mm*5 um; mobile phase: [H₂O (0.2% FA)-ACN]; gradient: 15%-45% B over 8.0 min) to give title Compound (206-3) (157 mg, 391 μmol, 3.69% yield) as a white solid. LCMS (ES⁺): m/z 402.2 [M+H]⁺.

Synthesis of trans-4-((5-fluoro-4-(2-(2-oxooxazolidin-3-yl)pyridin-4-yl)pyrimidin-2-yl)amino)cyclohexane-1-carboxamide (Compound 263). To a solution of trans-4-((5-fluoro-4-(2-(2-oxooxazolidin-3-yl)pyridin-4-yl)pyrimidin-2-yl)amino)cyclohexane-1-carboxylic acid (206-3) (137 mg, 341 μmol, 1.00 eq) in DMF (5 mL) were added NH₄Cl (27.4 mg, 512 μmol, 1.50 eq), DIEA (132 mg, 1.02 mmol, 178 μL, 3.00 eq) and HATU (195 mg, 512 μmol, 1.50 eq) at 15 °C. The mixture was stirred at 15 °C for 1 h. LC-MS showed that trans-4-((5-fluoro-4-(2-(2-oxooxazolidin-3-yl)pyridin-4-yl)pyrimidin-2-yl)amino)cyclohexane-1-carboxylic acid (206-3) was consumed completely and the desired mass was detected. The reaction mixture was concentrated under reduced pressure. The residue was purified by prep-HPLC (column: Phenomenex luna C18 100*40 mm*3 um; mobile phase: [H₂O (0.2% FA)-ACN]; gradient: 10%-40% B over 8.0 min) to give title compound (Compound 263) (31.6 mg, 77.7 μmol, 22.8% yield, 98.5% purity) as a yellow solid. LCMS (ES⁺): m/z 401.2 [M+H]⁺.

### Example 207. Synthesis of trans-N-(-4-((5-fluoro-4-(2-(2-oxopiperidin-1-yl)pyridin-4-yl)pyrimidin-2-yl)amino)cyclohexyl)acetamide (Compound 257)

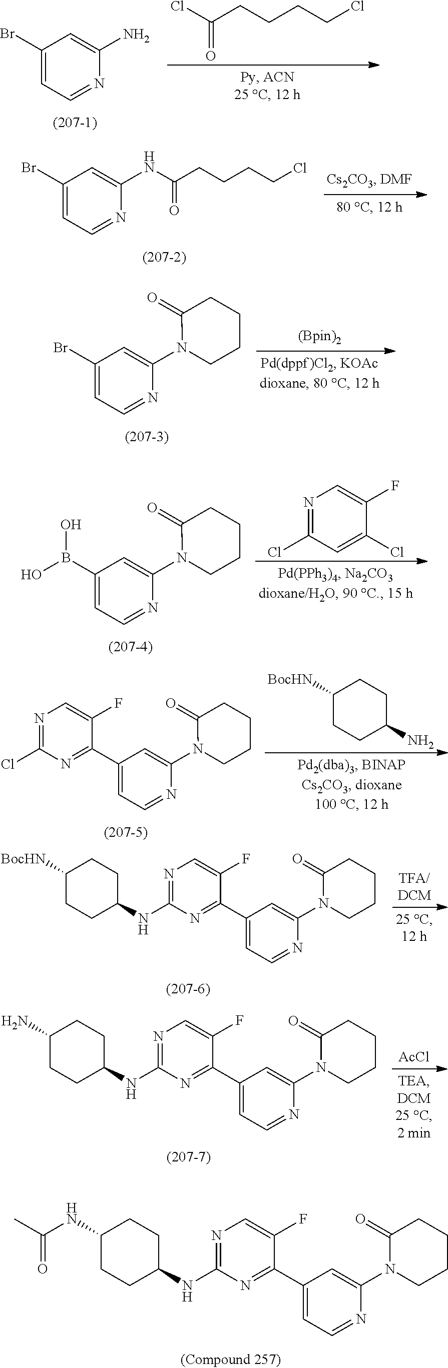

Synthesis of N-(4-bromopyridin-2-yl)-5-chloropentanamide (257-2). To a solution of 4-bromopyridin-2-amine (257-1) (10.0 g, 57.8 mmol, 1.00 eq) in ACN (150 mL) were added 5-chloropentanoyl chloride (10.8 g, 69.4 mmol, 8.96 mL, 1.20 eq) and Py. (13.7 g, 173 mmol, 14.0 mL, 3.00 eq) at 25 °C. The mixture was stirred at 25 °C for 12 h. LC-MS showed 4-bromopyridin-2-amine (257-1) was consumed completely, and the desired mass was detected. The reaction mixture was concentrated under reduced pressure to give the title compound (257-2) (16.9 g, crude) as a yellow oil. LCMS (ES⁺): m/z 291.0 [M+H]⁺.

Synthesis of 1-(4-bromopyridin-2-yl)piperidin-2-one (257-3). To a solution of N-(4-bromopyridin-2-yl)-5-chloropentanamide (257-2) (16.9 g, 57.8 mmol, 1.00 eq) in DMF (150 mL) was added Cs₂CO₃ (113 g, 347 mmol, 6.00 eq) at 25 °C. The mixture was stirred at 80 °C for 12 h. LC-MS showed N-(4-bromopyridin-2-yl)-5-chloropentanamide (257-2) was consumed completely and the desired mass was detected. water (150 mL) was added to the mixture at 25 °C. The reaction mixture was extracted with DCM (100 mL*5). The organic layers were dried over anhydrous sodium sulfate, filtered and the filtrate was concentrated under reduced to give the title compound (257-3) (16.7 g, crude) as a brown oil. LCMS (ES⁺): m/z 255.0 [M+H]⁺.

Synthesis of (2-(2-oxopiperidin-1-yl)pyridin-4-yl)boronic acid (257-4). To a solution of 1-(4-bromopyridin-2-yl)piperidin-2-one (257-3) (4.00 g, 15.7 mmol, 1.00 eq) in dioxane (100 mL) were added 4,4,5,5-tetramethyl-2-(4,4,5,5-tetramethyl-1,3,2-dioxaborolan-2-yl)-1,3,2-dioxaborolane (11.9 g, 47.0 mmol, 3.00 eq), KOAc (6.16 g, 62.7 mmol, 4.00 eq) and Pd(dppf)Cl₂ (1.15 g, 1.57 mmol, 0.100 eq) at 25 °C under N₂ atmosphere. The mixture was stirred at 80 °C for 12 h under N₂. LC-MS showed 1-(4-bromopyridin-2-yl)piperidin-2-one (257-3) was consumed completely and the desired mass was detected. The reaction mixture was concentrated under reduced pressure to give the title compound (257-4) (6.90 g, crude) as a brown solid. LCMS (ES⁺): m/z 221.1 [M+H]⁺.

Synthesis of 1-(4-(2-chloro-5-fluoropyrimidin-4-yl)pyridin-2-yl)piperidin-2-one (257-5). To a solution of (2-(2-oxopiperidin-1-yl)pyridin-4-yl)boronic acid (257-4) (6.90 g, 31.4 mmol, 1.00 eq) in dioxane (200 mL) and H₂O (20 mL) were added 2,4-dichloro-5-fluoropyrimidine (5.24 g, 31.4 mmol, 1.00 eq), Na₂CO₃ (6.65 g, 62.7 mmol, 2.00 eq) and Pd(PPh₃)₄ (725 mg, 627 μmol, 0.0200 eq) at 25 °C under N₂ atmosphere. The mixture was stirred at 90 °C for 3 h under N₂. LC-MS showed ~13.5% of (2-(2-oxopiperidin-1-yl)pyridin-4-yl)boronic acid (257-4) was remaining, and ~44.2% of the desired mass was detected. The mixture was stirred at 90 °C for another 12 h. LC-MS showed (2-(2-oxopiperidin-1-yl)pyridin-4-yl)boronic acid (257-4) was remaining little, and the desired mass was detected. Water (100 mL) was added to the mixture, then it was extracted with EtOAc (80 mL*3), the combined organic layers were dried over Na₂SO₄, filtered and the filtrate was concentrated under reduced pressure to give a residue. The residue was purified by column chromatography (SiO₂, Petroleum ether:Ethyl acetate=1:1 to 1:2) to give the title compound (257-5) (5.26 g, 17.2 mmol, 54.7% yield) as a brown solid. LCMS (ES⁺): m/z 307.1 [M+H]⁺.

Synthesis of trans-tert-butyl (4-((5-fluoro-4-(2-(2-oxopiperidin-1-yl)pyridin-4-yl)pyrimidin-2-yl)amino)cyclohexyl)carbamate (257-6). To a solution of 1-(4-(2-chloro-5-fluoropyrimidin-4-yl)pyridin-2-yl)piperidin-2-one (257-5) (1.00 g, 3.26 mmol, 1.00 eq) in dioxane (16 mL) were added trans-tert-butyl (4-aminocyclohexyl)carbamate (699 mg, 3.26 mmol, 1.00 eq), Cs₂CO₃ (3.19 g, 9.78 mmol, 3.00 eq), BINAP (203 mg, 326 μmol, 0.100 eq) and Pd₂(dba)₃ (299 mg, 326 μmol, 0.100 eq) at 25 °C under N₂ atmosphere. The mixture was stirred at 100 °C for 12 h under N₂. LC-MS showed 1-(4-(2-chloro-5-fluoropyrimidin-4-yl)pyridin-2-yl)piperidin-2-one (257-5) was consumed completely and the desired mass was detected. Water (15 mL) was added to the mixture at 25 °C. The reaction mixture was extracted with EtOAc (10 mL*5). The combined organic layers were dried over anhydrous sodium sulfate, filtered and the filtrate was concentrated under reduced to give a residue. The residue was purified by column chromatography (SiO₂, Petroleum ether:Ethyl acetate=2:1 to 1:1) to give the title compound (257-6) (1.00 g, 2.06 mmol, 63.3% yield) as a yellow solid. LCMS (ES⁺): m/z 485.3 [M+H]⁺.

Synthesis of trans-1-(4-(2-((4-aminocyclohexyl)amino)-5-fluoropyrimidin-4-yl)pyridin-2-yl)piperidin-2-one (257-7). To a solution of trans-tert-butyl (4-((5-fluoro-4-(2-(2-oxopiperidin-1-yl)pyridin-4-yl)pyrimidin-2-yl)amino)cyclohexyl)carbamate (257-6) (500 mg, 1.03 mmol, 1.00 eq) in DCM (4 mL) was added TFA (1.54 g, 13.5 mmol, 1.00 mL, 13.1 eq) at 25 °C. The mixture was stirred at 25 °C for 12 h. LC-MS showed trans-tert-butyl (4-((5-fluoro-4-(2-(2-oxopiperidin-1-yl)pyridin-4-yl)pyrimidin-2-yl)amino)cyclohexyl)carbamate (257-6) was consumed completely and the desired mass was detected. The reaction mixture was concentrated under reduced pressure to give the title compound (257-7) (650 mg, crude, TFA) as a yellow oil. LCMS (ES⁺): m/z 385.3 [M+H]⁺.

Synthesis of trans-N-(4-((5-fluoro-4-(2-(2-oxopiperidin-1-yl)pyridin-4-yl)pyrimidin-2-yl)amino)cyclohexyl)acetamide (Compound 257). To a solution of trans-1-(4-(2-((4-aminocyclohexyl)amino)-5-fluoropyrimidin-4-yl)pyridin-2-yl)piperidin-2-one (257-7) (550 mg, 1.10 mmol, 1.00 eq, TFA) in DCM (6 mL) was added TEA (335 mg, 3.31 mmol, 461 μL, 3.00 eq) at 25 °C. The mixture was added acetyl chloride (69.3 mg, 883 μmol, 62.8 μL, 0.800 eq) at 0 °C. The resulting mixture was stirred at 25 °C for 5 min. LC-MS showed trans-1-(4-(2-((4-aminocyclohexyl)amino)-5-fluoropyrimidin-4-yl)pyridin-2-yl)piperidin-2-one (257-7) was consumed completely and the desired mass was detected. Water (0.3 mL) was added to the mixture at 25 °C. The reaction mixture was concentrated under reduced pressure to give a residue. The residue was purified by prep-HPLC (column: Phenomenex Luna C18 100*30 mm*3 um; mobile phase: [1H₂O (0.2% FA)-ACN]; gradient: 20%-50% B over 8.0 min) to give the title compound (Compound 257) (111 mg, 258 μmol, 23.3% yield, 99.0% purity) as a light yellow solid. LCMS (ES⁺): m/z 427.1 [M+H]⁺.

### Example 208. Synthesis of trans-4-((5-fluoro-4-(2-(2-oxopiperidin-1-yl)pyridin-4-yl)pyrimidin-2-yl)amino)cyclohexane-1-carboxamide (Compound 258)

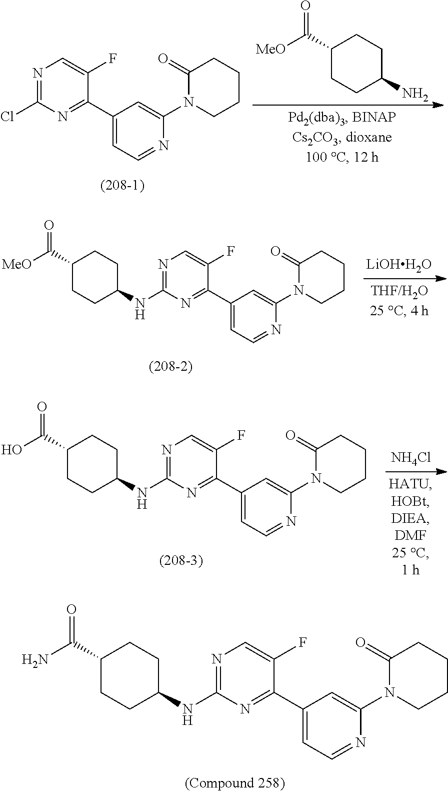

Synthesis of trans-methyl-4-((5-fluoro-4-(2-(2-oxopiperidin-1-yl)pyridin-4-yl)pyrimidin-2-yl)amino)cyclohexane-1-carboxylate (208-2). To a solution of 1-(4-(2-chloro-5-fluoropyrimidin-4-yl)pyridin-2-yl)piperidin-2-one (208-1) (1.00 g, 3.26 mmol, 1.00 eq) in dioxane (15 mL) were added trans-methyl-4-aminocyclohexane-1-carboxylate (513 mg, 3.26 mmol, 1.00 eq), Cs₂CO₃ (3.19 g, 9.78 mmol, 3.00 eq), BINAP (203 mg, 326 μmol, 0.100 eq) and Pd₂(dba)₃ (299 mg, 326 μmol, 0.100 eq) at 25 °C under N₂ atmosphere. The mixture was stirred at 100 °C for 12 h under N₂. LC-MS showed 1-(4-(2-chloro-5-fluoropyrimidin-4-yl)pyridin-2-yl)piperidin-2-one (208-1) was consumed completely and the desired mass was detected. Water (15 mL) was added to the mixture at 25 °C. The reaction mixture was extracted with EtOAc (10 mL*5). The organic layers were dried over anhydrous sodium sulfate, filtered and the filtrate was concentrated under reduced to give a residue. The residue was purified by column chromatography (SiO₂, Petroleum ether:Ethyl acetate=2:1 to 1:1) to give the title compound (208-2) (750 mg, 1.75 mmol, 53.8% yield) as a yellow oil. LCMS (ES⁺): m/z 428.2 [M+H]⁺.

Synthesis of trans-4-((5-fluoro-4-(2-(2-oxopiperidin-1-yl)pyridin-4-yl)pyrimidin-2-yl)amino)cyclohexane-1-carboxylic acid (208-3). To a solution of trans-methyl-4-((5-fluoro-4-(2-(2-oxopiperidin-1-yl)pyridin-4-yl)pyrimidin-2-yl)amino)cyclohexane-1-carboxylate (208-2) (190 mg, 444 μmol, 1.00 eq) in THF (2 mL) and H₂O (0.4 mL) was added LiOH·H₂O (56.0 mg, 1.33 mmol, 3.00 eq) at 25 °C. The mixture was stirred at 25 °C for 2 h. LC-MS showed ~70.5% of trans-methyl-4-((5-fluoro-4-(2-(2-oxopiperidin-1-yl)pyridin-4-yl)pyrimidin-2-yl)amino)cyclohexane-1-carboxylate (208-2) was remaining, and ~14.9% of the desired mass was detected. The mixture was stirred at 25 °C for another 2 h. LC-MS showed trans-methyl-4-((5-fluoro-4-(2-(2-oxopiperidin-1-yl)pyridin-4-yl)pyrimidin-2-yl)amino)cyclohexane-1-carboxylate (208-2) was remaining little, and the desired mass was detected. The reaction was concentrated under reduced to remove THF, then water (2 mL) was added to the mixture at 25 °C. HCl (1 M) was added to the aqueous phase to adjust pH to 2-3. Then the above aqueous phase was extracted with EtOAC (4 mL*5). The combined organic layers were dried over anhydrous sodium sulfate, filtered and the filtrate was concentrated under reduced to give the title compound (208-3) (109 mg, 264 μmol, 59.3% yield) as a yellow oil. LCMS (ES⁺): m/z 414.2 [M+H]⁺.

Synthesis of trans-4-((5-fluoro-4-(2-(2-oxopiperidin-1-yl)pyridin-4-yl)pyrimidin-2-yl)amino)cyclohexane-1-carboxamide (Compound 258). To a solution of trans-4-((5-fluoro-4-(2-(2-oxopiperidin-1-yl)pyridin-4-yl)pyrimidin-2-yl)amino)cyclohexane-1-carboxylic acid (208-3) (109 mg, 264 μmol, 1.00 eq) in DMF (2 mL) were added HATU (121 mg, 316 μmol, 1.20 eq), HOBt (17.8 mg, 132 μmol, 0.500 eq), DIEA (171 mg, 1.32 mmol, 230 μL, 5.00 eq) and NH₄Cl (56.4 mg, 1.05 mmol, 4.00 eq) at 25 °C. The mixture was stirred at 25 °C for 1 h. LC-MS showed trans-4-((5-fluoro-4-(2-(2-oxopiperidin-1-yl)pyridin-4-yl)pyrimidin-2-yl)amino)cyclohexane-1-carboxylic acid (208-3) was consumed completely and the desired mass was detected. The reaction mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 10%-50% B over 8.0 min) to give the title compound (Compound 258) (35.7 mg, 86.6 μmol, 32.8% yield, 100% purity) as a yellow solid. LCMS (ES⁺): m/z 413.1 [M+H]+

### Example 209. Synthesis of trans-N-(4-((5-fluoro-4-(2-oxo-2H-[1,2′-bipyridin]-4′-yl)pyrimidin-2-yl)amino)cyclohexyl)acetamide (Compound 265)

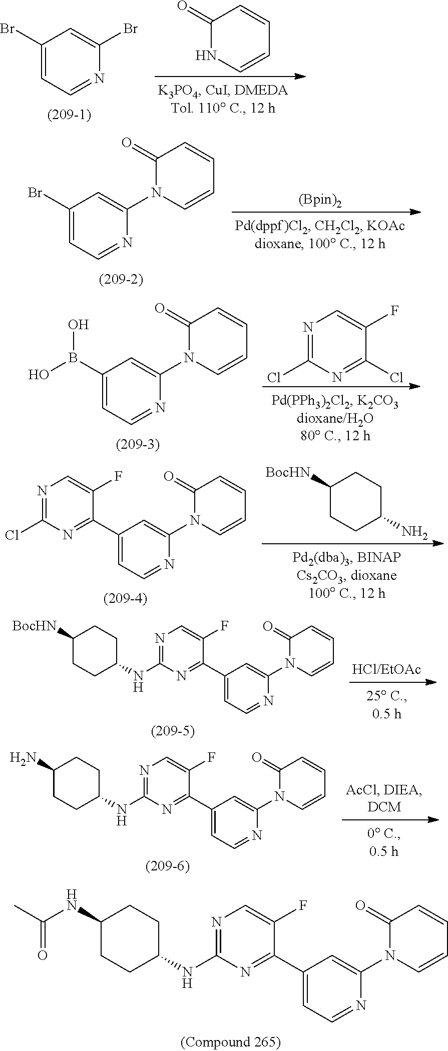

Synthesis of 4′-bromo-2H-[1,2′-bipyridin]-2-one (209-2). To a solution of 2,4-dibromopyridine (209-1) (5.00 g, 21.1 mmol, 1.00 eq) and pyridin-2(1H)-one (2.01 g, 21.1 mmol, 1.00 eq) in Tol. (100 mL) were added K₃PO₄ (8.96 g, 42.2 mmol, 2.00 eq), CuI (804 mg, 4.22 mmol, 0.200 eq) and DMEDA (372 mg, 4.22 mmol, 454 μL, 0.200 eq) at 25 °C under N₂. The mixture was stirred at 110 °C for 12 h under N₂. LCMS showed 2,4-dibromopyridine (209-1) was consumed and the desired mass was detected. To the mixture was added H₂O (100 mL) and the mixture was extracted with EtOAc (80 mL*3). The combined organic layers were washed with brine (50 mL*3), dried over Na₂SO₄, filtered and the filtrate was concentrated under reduced pressure to give a residue. The residue was purified by MPLC (SiO₂, PE/EtOAc=1/0 to 1/1) to give the title compound (209-2) (2.60 g, 10.4 mmol, 49.1% yield) as white solid. LCMS (ES⁺): m/z 251.0 [M+H]⁺.

Synthesis of (2-oxo-2H-[1,2′-bipyridin]-4′-yl)boronic acid (209-3). To a solution of 4′-bromo-2H-[1,2′-bipyridin]-2-one (209-2) (2.60 g, 10.4 mmol, 1.00 eq) and 4,4,5,5-tetramethyl-2-(4,4,5,5-tetramethyl-1,3,2-dioxaborolan-2-yl)-1,3,2-dioxaborolane (3.42 g, 13.5 mmol, 1.30 eq) in dioxane (140 mL) were added KOAc (3.05 g, 31.1 mmol, 3.00 eq) and Pd(dppf)Cl₂·CH₂Cl₂ (846 mg, 1.04 mmol, 0.100 eq) at 20 °C under N. The mixture was stirred at 100 °C for 12 h under N₂. LCMS showed 4′-bromo-2H-[1,2′-bipyridin]-2-one (209-2) was consumed and the desired mass was detected. The mixture was used for the next step directly to give the title compound (209-3) (2.24 g, crude) in dioxane (140 mL) as a brown liquid. LCMS (ES⁺): m/z 217.1 [M+H]⁺.

Synthesis of 4′-(2-chloro-5-fluoropyrimidin-4-yl)-2H-[1,2′-bipyridin]-2-one (209-4). To a solution of (2-oxo-2H-[1,2′-bipyridin]-4′-yl)boronic acid (209-3) (2.24 g, 10.4 mmol, 1.00 eq) and 2,4-dichloro-5-fluoropyrimidine (2.60 g, 15.6 mmol, 1.50 eq) in dioxane (140 mL) and H₂O (14 mL) were added K₂CO₃ (4.30 g, 31.1 mmol, 3.00 eq) and Pd(PPh₃)₂Cl₂ (72.8 mg, 104 μmol, 0.0100 eq) at 25 °C under N₂. The mixture was stirred at 80 °C for 12 h under N₂. LCMS showed (2-oxo-2H-[1,2′-bipyridin]-4′-yl)boronic acid (209-3) was consumed and the desired mass was detected. To the mixture was added H₂O (50 mL) and the mixture was extracted with EtOAc (50 mL*3). The combined organic layers were washed with brine (10 mL*3), dried over Na₂SO₄, filtered and the filtrate was concentrated under reduced pressure to give a residue. The residue was purified by MPLC (SiO₂, PE/EtOAc=1/0 to 0/1) to give the title compound (209-4) (1.68 g, 5.55 mmol, 53.5% yield) as a yellow solid. LCMS (ES⁺): m/z 303.0 [M+H]⁺.

Synthesis of trans-tert-butyl (4-((5-fluoro-4-(2-oxo-2H-[1,2′-bipyridin]-4′-yl)pyrimidin-2-yl)amino)cyclohexyl)carbamate (209-5). To a solution of 4′-(2-chloro-5-fluoropyrimidin-4-yl)-2H-[1,2′-bipyridin]-2-one (209-4) (1.67 g, 5.52 mmol, 1.00 eq) and trans-tert-butyl (4-aminocyclohexyl)carbamate (1.77 g, 8.28 mmol, 1.50 eq) in dioxane (30 mL) were added BINAP (309 mg, 497 μmol, 0.0900 eq), Cs₂CO₃ (3.60 g, 11.0 mmol, 2.00 eq) and Pd₂ (dba)₃ (253 mg, 276 μmol, 0.0500 eq) at 25 °C under N₂. The mixture was stirred at 100 °C for 12 h under N₂ atmosphere. LCMS showed 4′-(2-chloro-5-fluoropyrimidin-4-yl)-2H-[1,2′-bipyridin]-2-one (209-4) was consumed and the desired mass was detected. To the mixture was added H₂O (30 mL) and the mixture was extracted with EtOAc (30 mL*3). The combined organic layers were washed with brine (30 mL*3), dried over Na₂SO₄, filtered and the filtrate was concentrated under reduced pressure to give a residue. The residue was purified by MPLC (SiO₂, PE/EtOAc=1/0 to 1/1) to give the title compound (209-5) (2.00 g, 4.16 mmol, 75.4% yield) as a yellow solid. LCMS (ES⁺): m/z 481.2 [M+H]⁺.

Synthesis of trans-4′-(2-((4-aminocyclohexyl)amino)-5-fluoropyrimidin-4-yl)-2H-[1,2′-bipyridin]-2-one (209-6). A solution of trans-tert-butyl (4-((5-fluoro-4-(2-oxo-2H-[1,2′-bipyridin]-4′-yl)pyrimidin-2-yl)amino)cyclohexyl)carbamate (209-5) (500 mg, 1.04 mmol, 1.00 eq) in HCl/EtOAc (5 mL) was stirred at 25 °C for 0.5 h. LCMS showed trans-tert-butyl (4-((5-fluoro-4-(2-oxo-2H-[1,2′-bipyridin]-4′-yl)pyrimidin-2-yl)amino)cyclohexyl)carbamate (209-5) was consumed and the desired mass was detected. The mixture was concentrated under reduced pressure to give the title compound (209-6) (434 mg, crude, HCl) as a yellow oil. LCMS (ES⁺): m/z 381.2 [M+H]⁺.

Synthesis of trans-N-(4-((5-fluoro-4-(2-oxo-2H-[1,2′-bipyridin]-4′-yl)pyrimidin-2-yl)amino)cyclohexyl)acetamide (Compound 265). To a solution of trans-4′-(2-((4-aminocyclohexyl)amino)-5-fluoropyrimidin-4-yl)-2H-[1,2′-bipyridin]-2-one (209-6) (434 mg, 1.04 mmol, 1.00 eq, HCl) in DCM (4 mL) were added DIEA (404 mg, 3.12 mmol, 544 L, 3.00 eq) at 25 °C. Then to the mixture was added AcCl (65.4 mg, 833 μmol, 59.2 μL, 0.800 eq) at 0 °C. The mixture was stirred at 0 °C for 0.5 h. LCMS showed trans-4′-(2-((4-aminocyclohexyl)amino)-5-fluoropyrimidin-4-yl)-2H-[1,2′-bipyridin]-2-one (209-6) was consumed and the desired mass was detected. H₂O (0.3 mL) was added to the mixture to quench AcCl. Then the mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 10%-50% B over 8.0 min) to give the title compound (Compound 265) (243.6 mg, 573 μmol, 55.0% yield, 99.4% purity) as a yellow solid. LCMS (ES⁺): m/z 423.2 [M+H]⁺.

### Example 210. Synthesis of trans-4-((5-fluoro-4-(2-oxo-2H-[1,2′-bipyridin]-4′-yl)pyrimidin-2-yl)amino)cyclohexane-1-carboxamide (Compound 623)

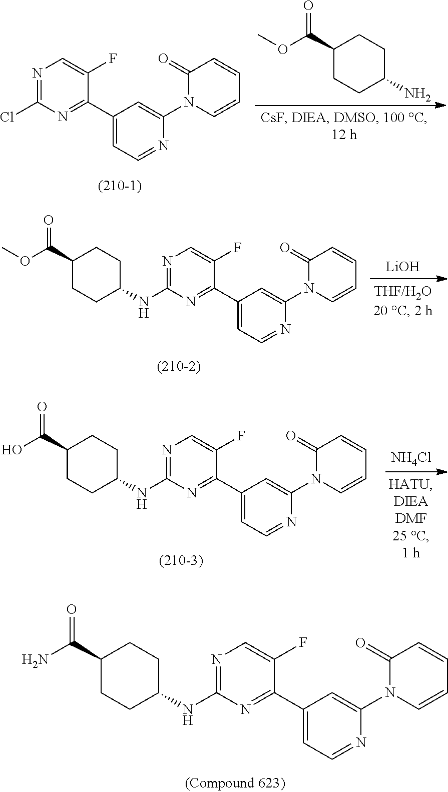

Synthesis of methyl-trans-4-((5-fluoro-4-(2-oxo-2H-[1,2′-bipyridin]-4′-yl)pyrimidin-2-yl)amino)cyclohexane-1-carboxylate (210-2). A mixture of 4′-(2-chloro-5-fluoropyrimidin-4-yl)-2H-[1,2′-bipyridin]-2-one (210-1) (3.00 g, 9.91 mmol, 1.00 eq), methyl 4-aminocyclohexanecarboxylate (2.34 g, 14.8 mmol, 1.50 eq), CsF (3.01 g, 19.8 mmol, 731 L, 2.00 eq), DIEA (2.56 g, 19.8 mmol, 3.45 mL, 2.00 eq) in DMSO (30 mL) was degassed and purged with N₂ at 25 °C for 3 times, and then the mixture was stirred at 100 °C for 12 h under N₂ atmosphere. LC-MS showed the desired mass was detected. The reaction mixture was quenched by addition H₂O 30 mL, and then diluted with H₂O 30 mL and extracted with EtOAc (30 mL*3). The combined organic layers were washed with sat.aq. NaCl (10 mL), dried over Na₂SO₄, filtered and concentrated under reduced pressure to give a residue. The residue was purified by column chromatography (SiO₂, Petroleum ether:Ethyl acetate=1:0 to 1:1) to give the title compound (3.10 g, 5.87 mmol, 59.2% yield, 80.2% purity) as a yellow solid. LCMS (ES⁺): m/z 424.2 [M+H]⁺.

Synthesis of trans-4-((5-fluoro-4-(2-oxo-2H-[1,2′-bipyridin]-4′-yl)pyrimidin-2-yl)amino)cyclohexane-1-carboxylic acid. To a solution of methyl-trans-4-((5-fluoro-4-(2-oxo-2H-[1,2′-bipyridin]-4′-yl)pyrimidin-2-yl)amino)cyclohexane-1-carboxylate (2.00 g, 4.72 mmol, 1.00 eq) in THE (20 mL) and H₂O (5 mL) was added LiOH (339 mg, 14.1 mmol, 3.00 eq) at 20 °C. The mixture was stirred at 20 °C for 2 h. LC-MS showed the desired mass was detected. The mixture was adjust PH to 5 with 1M HCl and extracted with EtOAc 30 mL*3 and concentrated under reduced pressure to give the title compound (1.40 g, crude) as a yellow solid. LCMS (ES⁺): m/z 410.1 [M+H]⁺.

Synthesis of trans-4-((5-fluoro-4-(2-oxo-2H-[1,2′-bipyridin]-4′-yl)pyrimidin-2-yl)amino)cyclohexane-1-carboxamide (Compound 623). To a solution of 4-[[5-fluoro-4-[2-(2-oxo-1-pyridyl)-4-pyridyl]pyrimidin-2-yl]amino]cyclohexanecarboxylic acid (210-2) (90.0 mg, 219 μmol, 1.00 eq) in DMF (1 mL) was added NH₄Cl (12.9 mg, 241 μmol, 1.10 eq), DIEA (28.4 mg, 219 μmol, 38.2 μL, 1.00 eq) and HATU (83.5 mg, 219 μmol, 1.00 eq) at 25 °C. The mixture was stirred at 25 °C for 1 h. LC-MS showed the desired mass was detected. The reaction mixture was quenched by addition H₂O 10 mL, and then extracted with EtOAc (10 mL*3). The combined organic layers were dried over Na₂SO₄, filtered and concentrated under reduced pressure. The residue was purified by prep-HPLC (column: Phenomenex luna C18 100*40 mm*3 um; mobile phase: [H₂O (0.2% FA)-ACN]; gradient: 10%-40% B over 8.0 min) to give the title compound (Compound 623) (20.2 mg, 49.4 μmol, 22.5% yield, 99.5% purity) as a yellow solid. LCMS (ES⁺): m/z 409.2 [M+H]⁺.

### Example 211. Synthesis of trans-N-(4-((5-fluoro-4-(2-(2-oxo-1,3-oxazinan-3-yl)pyridin-4-yl)pyrimidin-2-yl)amino)cyclohexyl)acetamide (Compound 266)

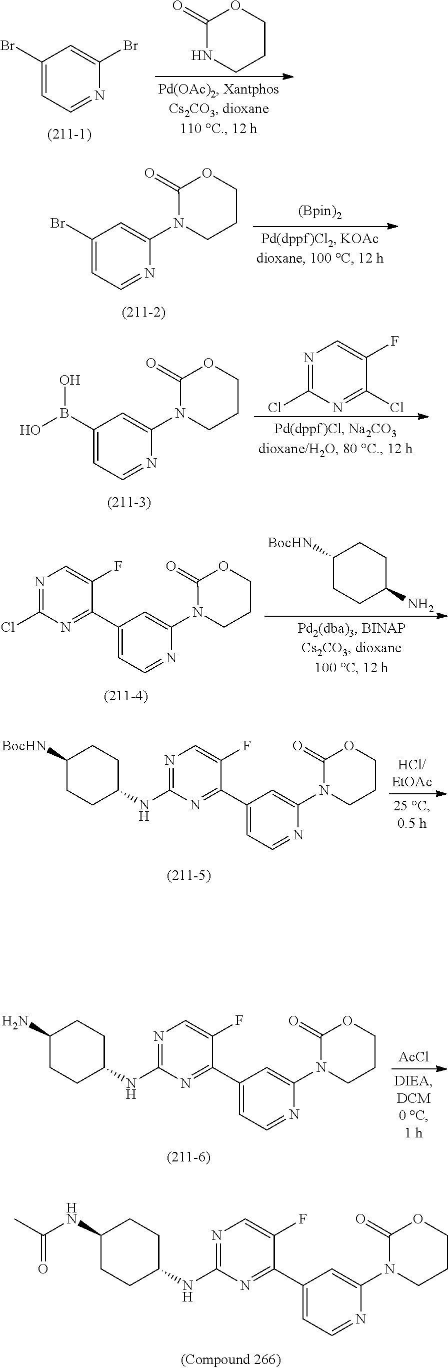

Synthesis of 3-(4-bromopyridin-2-yl)-1,3-oxazinan-2-one (211-2). A mixture of 2,4-dibromopyridine (211-1) (40.0 g, 169 mmol, 1.50 eq), 1,3-oxazinan-2-one (11.4 g, 113 mmol, 1.00 eq), Xantphos (5.21 g, 9.01 mmol, 0.0800 eq), Cs₂CO₃ (73.4 g, 225 mmol, 2.00 eq) and Pd(OAc)₂ (2.53 g, 11.3 mmol, 0.100 eq) in dioxane (1000 mL) was degassed and purged with N₂ for 3 times at 20 °C, and then the mixture was stirred at 110 °C for 12 h under N₂ atmosphere. LCMS showed 2,4-dibromopyridine (211-1) was consumed and the desired mass was detected. To the mixture was added H₂O (1000 mL) and the mixture was extracted with EtOAc (800 mL*3). The combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (211-2) (13.0 g, 50.6 mmol, 44.9% yield) as a yellow oil. LCMS (ES⁺): m/z 256.9 [M+H]⁺.

Synthesis of (2-(2-oxo-1,3-oxazinan-3-yl)pyridin-4-yl)boronic acid (211-3). To a solution of 3-(4-bromopyridin-2-yl)-1,3-oxazinan-2-one (211-2) (13.0 g, 50.6 mmol, 1.00 eq) and 4,4,5,5-tetramethyl-2-(4,4,5,5-tetramethyl-1,3,2-dioxaborolan-2-yl)-1,3,2-dioxaborolane (25.7 g, 101 mmol, 2.00 eq) in dioxane (200 mL) were added KOAc (9.93 g, 101 mmol, 2.00 eq) and Pd(dppf)Cl₂ (3.70 g, 5.06 mmol, 0.100 eq) at 20 °C under N₂. The mixture was stirred at 100 °C for 12 h under N₂. LCMS showed 3-(4-bromopyridin-2-yl)-1,3-oxazinan-2-one (211-2) was consumed and the desired mass was detected. The mixture was concentrated under reduced pressure to give the title compound (211-3) (15.4 g, crude) as a black oil without further work up and purification. LCMS (ES⁺): m/z 223.1[M+H]⁺.

Synthesis of 3-(4-(2-chloro-5-fluoropyrimidin-4-yl)pyridin-2-yl)-1,3-oxazinan-2-one (211-4). A mixture of (2-(2-oxo-1,3-oxazinan-3-yl)pyridin-4-yl)boronic acid (211-3) (800 mg, 2.63 mmol, 1.00 eq) and 2,4-dichloro-5-fluoropyrimidine (878 mg, 5.26 mmol, 2.00 eq), Pd(dppf)Cl₂ (192 mg, 263 μmol, 0.100 eq) and Na₂CO₃ (558 mg, 5.26 mmol, 2.00 eq) in dioxane (15 mL) and H₂O (3 mL) was degassed and purged with N₂ for 3 times at 20 °C, and then the mixture was stirred at 80 °C for 12 h under N₂ atmosphere. LCMS showed that the (2-(2-oxo-1,3-oxazinan-3-yl)pyridin-4-yl)boronic acid (211-3) was consumed and the desired mass was detected. To the mixture was added H₂O (15 mL) and the mixture was extracted with EtOAc (10 mL*3). The combined organic layers were dried over Na₂SO₄, filtered and the filtrate was concentrated under reduced pressure to give a residue. The residue was purified by prep-TLC (SiO₂, PE/EtOAc=0/1) to give the title compound (211-4) (400 mg, 1.30 mmol, 49.3% yield) as a yellow oil. LCMS (ES⁺): m/z 308.9 [M+H]⁺.

Synthesis of trans-tert-butyl (4-((5-fluoro-4-(2-(2-oxo-1,3-oxazinan-3-yl)pyridin-4-yl)pyrimidin-2-yl)amino)cyclohexyl)carbamate (211-5). To a solution of 3-(4-(2-chloro-5-fluoropyrimidin-4-yl)pyridin-2-yl)-1,3-oxazinan-2-one (211-4) (400 mg, 1.30 mmol, 1.00 eq) and trans-tert-butyl (4-aminocyclohexyl)carbamate (417 mg, 1.94 mmol, 1.50 eq) in dioxane (10 mL) were added Pd₂(dba)₃ (71.2 mg, 77.8 μmol, 0.0600 eq), BINAP (64.6 mg, 103 μmol, 0.0800 eq) and Cs₂CO₃ (844 mg, 2.59 mmol, 2.00 eq) at 25 °C under N₂, then the mixture was stirred at 100 °C for 12 h under N₂ atmosphere. LCMS showed that the 3-(4-(2-chloro-5-fluoropyrimidin-4-yl)pyridin-2-yl)-1,3-oxazinan-2-one (211-4) was consumed and the desired mass was detected. To the mixture was added H₂O (8 mL) and the mixture was extracted with EtOAc (5 mL*3). The combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (211-5) (300 mg, 617 μmol, 47.6% yield) as a brown oil. LCMS (ES⁺): m/z 487.3 [M+H]⁺.

Synthesis of trans-3-(4-(2-((4-aminocyclohexyl)amino)-5-fluoropyrimidin-4-yl)pyridin-2-yl)-1,3-oxazinan-2-one (211-6). A solution of trans tert-butyl (4-((5-fluoro-4-(2-(2-oxo-1,3-oxazinan-3-yl)pyridin-4-yl)pyrimidin-2-yl)amino)cyclohexyl)carbamate (211-5) (300 mg, 617 μmol, 1.00 eq) in HCl/EtOAc (4 mL) was stirred at 25 °C for 0.5 h. LCMS showed trans-tert-butyl (4-((5-fluoro-4-(2-(2-oxo-1,3-oxazinan-3-yl)pyridin-4-yl)pyrimidin-2-yl)amino)cyclohexyl)carbamate (211-5) was consumed completely and the desired mass was detected. The mixture was concentrated under reduced pressure to give the title compound (211-6) (260 mg, crude, HCl) as a yellow oil. LCMS (ES⁺): m/z 387.2 [M+H]⁺.

Synthesis of trans-N-(4-((5-fluoro-4-(2-(2-oxo-1,3-oxazinan-3-yl)pyridin-4-yl)pyrimidin-2-yl)amino)cyclohexyl)acetamide (Compound 266). To a solution of trans-3-(4-(2-((4-aminocyclohexyl)amino)-5-fluoropyrimidin-4-yl)pyridin-2-yl)-1,3-oxazinan-2-one (211-6) (260 mg, 615 μmol, 1.00 eq, HCl) in DCM (3 mL) was added DIEA (238 mg, 1.84 mmol, 321 μL, 3.00 eq) at 25 °C. Then to the reaction was added acetyl chloride (38.6 mg, 492 μmol, 35.0 μL, 0.800 eq) at 0 °C, The resulting mixture was stirred at 0 °C for 1 h. LCMS showed trans-3-(4-(2-((4-aminocyclohexyl)amino)-5-fluoropyrimidin-4-yl)pyridin-2-yl)-1,3-oxazinan-2-one (211-6) was consumed completely and the desired mass was detected. The mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 20%-55% B over 8.0 min) to give the title compound (Compound 266) (89.3 mg, 200 μmol, 32.6% yield, 96.1% purity) as a yellow solid. LCMS (ES⁺): m/z 429.1 [M+H]⁺.

### Example 212. Synthesis of trans-4-[[5-fluoro-4-[2-(2-oxo-1,3-oxazinan-3-yl)-4-pyridyl]pyrimidin-2-yl]amino]cyclohexanecarboxamide (Compound 622)

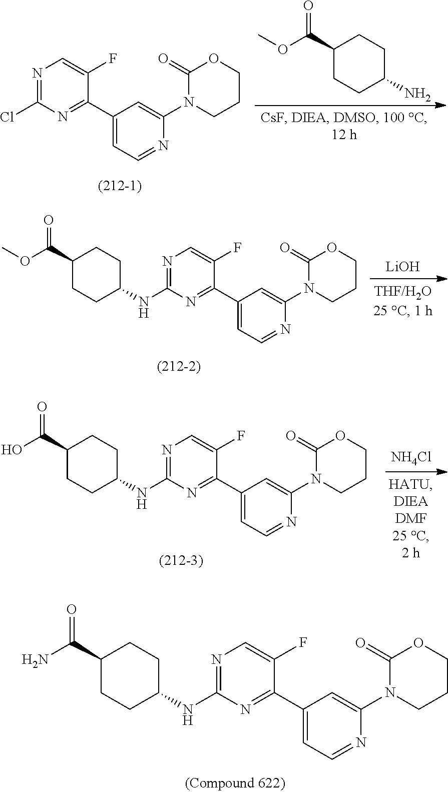

Synthesis of trans-methyl 4-((5-fluoro-4-(2-(2-oxo-1,3-oxazinan-3-yl)pyridin-4-yl)pyrimidin-2-yl)amino)cyclohexane-1-carboxylate (212-2). To a solution of 3-[4-(2-chloro-5-fluoro-pyrimidin-4-yl)-2-pyridyl]-1,3-oxazinan-2-one (212-1) (3.00 g, 9.72 mmol, 1.00 eq) in DMSO (20 mL) was added CsF (1.48 g, 9.72 mmol, 358 μL, 1.00 eq), trans-methyl 4-aminocyclohexane-1-carboxylate (2.29 g, 14.5 mmol, 1.50 eq) and DIEA (3.77 g, 29.1 mmol, 5.08 mL, 3.00 eq) at 25 °C. The mixture was stirred at 100 °C for 12 h. LC-MS showed 3-[4-(2-chloro-5-fluoro-pyrimidin-4-yl)-2-pyridyl]-1,3-oxazinan-2-one (212-1) was consumed completely and the desired mass was detected. The reaction mixture was quenched by addition H₂O 20 mL at 25 °C, and then diluted with H₂O 10 mL and then extracted with EtOAc (50 mL*3). The combined organic layers were washed with brine (30 mL*3), dried over Na₂SO₄, filtered and concentrated under reduced pressure. The residue was purified by column chromatography (SiO₂, Petroleum ether:Ethyl acetate=1:0 to 1:1) to give the title compound (212-2) (1.50 g, 3.42 mmol, 35.2% yield, 97.9% purity) as a brown solid. LCMS (ES⁺): m/z 430.1 [M+H]⁺.

Synthesis of trans-4-((5-fluoro-4-(2-(2-oxo-1,3-oxazinan-3-yl)pyridin-4-yl)pyrimidin-2-yl)amino)cyclohexane-1-carboxylic acid (212-3). To a solution of trans-methyl 4-((5-fluoro-4-(2-(2-oxo-1,3-oxazinan-3-yl)pyridin-4-yl)pyrimidin-2-yl)amino)cyclohexane-1-carboxylate (212-2) (400 mg, 931 μmol, 1.00 eq) in THF (5 mL) and H₂O (1 mL) was added LiOH (66.9 mg, 2.79 mmol, 3.00 eq) at 25 °C. The mixture was stirred at 25 °C for 1 h. LC-MS showed trans-methyl 4-((5-fluoro-4-(2-(2-oxo-1,3-oxazinan-3-yl)pyridin-4-yl)pyrimidin-2-yl)amino)cyclohexane-1-carboxylate (212-2) was consumed completely and the desired mass was detected. The reaction mixture was quenched by addition H₂O 10 mL at 25 °C, and then diluted with H₂O 10 mL, Adjust PH to 5, and then extracted with EtOAc (30 mL*3). The combined organic layers were washed with brine (30 mL*3), dried over Na₂SO₄, filtered and concentrated under reduced pressure to give the title compound (212-3) (700 mg, crude) as a brown solid. LCMS (ES⁺): m/z 416.2 [M+H]⁺.

Synthesis of trans-1-[3-[5-fluoro-2-[(4-hydroxycyclohexyl)amino]pyrimidin-4-yl]phenyl]pyridin-2-one (Compound 622). To a solution of 4-[[5-fluoro-4-[2-(2-oxo-1,3-oxazinan-3-yl)-4-pyridyl]pyrimidin-2-yl]amino]cyclohexanecarboxylic acid (212-1) (100.00 mg, 240.7 μmol, 1.00 eq) and NH₄Cl (64.4 mg, 1.20 mmol, 5.00 eq) in DMF (3 mL) was added HATU (137 mg, 361.1 μmol, 1.50 eq) and DIEA (93.3 mg, 722 μmol, 125 μL, 3.00 eq) at 25 °C. The mixture was stirred at 25 °C for 2 h. LC-MS showed 212-1 was consumed completely and the desired mass was detected. The reaction mixture was concentrated under reduced pressure. The residue was purified by prep-HPLC (column: Waters Xbridge BEH C18 100*30 mm*10 um; mobile phase: [H₂O (10 mM NH₄HCO₃)-ACN]; gradient: 10%-40% B over 8.0 min) to give the title compound (Compound 622) (3.8 mg, 9.17 μmol, 3.81% yield, 100% purity) as a white solid. LCMS (ES⁺): m/z 415.1 [M+H]⁺.

### Example 213. Synthesis of trans-4-((5-fluoro-4-(6-(2-oxopiperidin-1-yl)pyridin-2-yl)pyrimidin-2-yl)amino)cyclohexane-1-carboxamide (Compound 274)

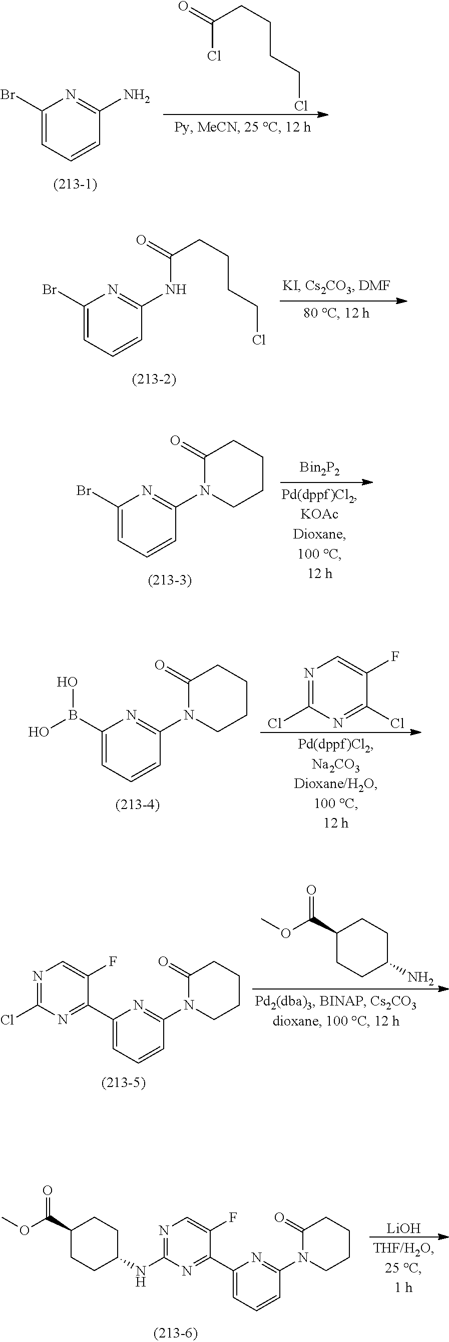

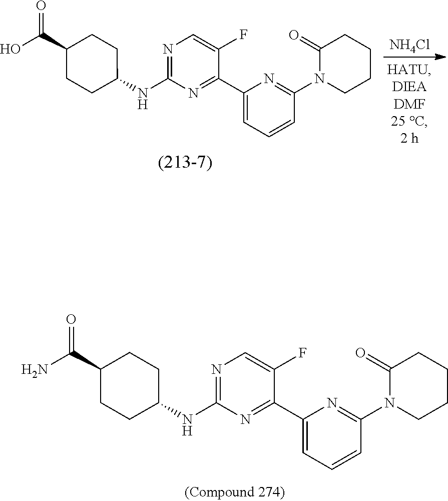

Synthesis of N-(6-bromo-2-pyridyl)-5-chloro-pentanamide (213-2). To a solution of 6-bromopyridin-2-amine (213-1) (20.0 g, 115 mmol, 1.00 eq) in MeCN (200 mL) was added Py (18.2 g, 231 mmol, 18.6 mL, 2.00 eq) and 5-chloropentanoyl chloride (17.9 g, 115 mmol, 14.9 mL, 1.00 eq) at 25 °C. The mixture was stirred at 25 °C for 12 h. LC-MS showed the desired mass was detected. The reaction mixture was diluted with water 100 mL and extracted with EtOAc (250 mL*3), filtered and concentrated under reduced pressure to give the title compound (213-2) (76.5 g, crude) as a brown oil. LCMS (ES⁺): m/z 291.0 [M+H]⁺.

Synthesis of 1-(6-bromo-2-pyridyl)piperidin-2-one (213-3). To a solution of N-(6-bromo-2-pyridyl)-5-chloro-pentanamide (213-2) (75.0 g, 257 mmol, 1.00 eq) in DMF (300 mL) was added Cs₂CO₃ (167 g, 514 mmol, 2.00 eq) and KI (85.4 g, 514 mmol, 2.00 eq) at 25 °C. The mixture was stirred at 80 °C for 12 h. LC-MS showed the desired mass was detected. The reaction mixture was diluted with water 50 mL and extracted with EtOAc (70 mL*3), filtered and concentrated under reduced pressure. The residue was purified by column chromatography (SiO₂, Petroleum ether:Ethyl acetate=1:0 to 1:1) to give the title compound (213-3) (45.0 g, 132 mmol, 51.5% yield, 75.1% purity) as a brown oil. LCMS (ES⁺): m/z 255.0 [M+H]⁺.

Synthesis of (6-(2-oxopiperidin-1-yl)pyridin-2-yl)boronic acid (213-4). A mixture of 1-(6-bromo-2-pyridyl)piperidin-2-one (213-3) (20.0 g, 78.4 mmol, 1.00 eq), 4,4,5,5-tetramethyl-2-(4,4,5,5-tetramethyl-1,3,2-dioxaborolan-2-yl)-1,3,2-dioxaborolane (19.9 g, 78.4 mmol, 1.00 eq), Pd(dppf)Cl₂ (5.74 g, 7.84 mmol, 0.100 eq), KOAc (23.0 g, 235 mmol, 3.00 eq) in dioxane (200 mL) at 25 °C was degassed and purged with N₂ for 3 times, and then the mixture was stirred at 100 °C for 12 h under N₂ atmosphere. LC-MS showed the desired mass was detected. The crude product (213-4) (crude) was used into the next step without further purification. LCMS (ES⁺): m/z 221.2 [M+H]⁺.

Synthesis of 1-[6-(2-chloro-5-fluoro-pyrimidin-4-yl)-2-pyridyl]piperidin-2-one (213-5). A mixture of (6-(2-oxopiperidin-1-yl)pyridin-2-yl)boronic acid (213-4) (20.0 g, 66.1 mmol, 1.00 eq), 2,4-dichloro-5-fluoro-pyrimidine (11.0 g, 66.1 mmol, 1.00 eq), Pd(dppf)Cl₂ (4.84 g, 6.62 mmol, 0.100 eq), Na₂CO₃ (21.0 g, 198 mmol, 3.00 eq) in dioxane (300 mL) and H₂O (100 mL) at 25 °C was degassed and purged with N₂ for 3 times, and then the mixture was stirred at 100 °C for 12 h under N₂ atmosphere. LC-MS showed the desired mass was detected. The reaction mixture was diluted with water 100 mL and extracted with EtOAc (300 mL*3), filtered and concentrated under reduced pressure. The residue was purified by column chromatography (SiO₂, Petroleum ether:Ethyl acetate=1:0 to 5:1) to give the title compound (213-5) (19.0 g, 41.7 mmol, 63.0% yield, 67.3% purity) as a yellow oil. LCMS (ES⁺): m/z 307.0 [M+H]⁺.

Synthesis of trans-4-[[5-fluoro-4-[6-(2-oxo-1-piperidyl)-2-pyridyl]pyrimidin-2-yl]amino]cyclohexanecarboxylate (213-6). A mixture of 1-[6-(2-chloro-5-fluoro-pyrimidin-4-yl)-2-pyridyl]piperidin-2-one (213-5) (10.0 g, 32.6 mmol, 1.00 eq), trans-methyl 4-aminocyclohexanecarboxylate (5.13 g, 32.6 mmol, 1.00 eq), Pd₂(dba)₃ (2.99 g, 3.26 mmol, 0.100 eq), BINAP (2.03 g, 3.26 mmol, 0.100 eq) and Cs₂CO₃ (31.8 g, 97.8 mmol, 3.00 eq) in dioxane (200 mL) at 25 °C was degassed and purged with N₂ for 3 times, and then the mixture was stirred at 100 °C for 12 h under N₂ atmosphere. LC-MS showed the desired mass was detected. The reaction mixture was diluted with water 150 mL and extracted with EtOAc (200 mL*3), filtered and concentrated under reduced pressure. The residue was purified by column chromatography (SiO₂, Petroleum ether:Ethyl acetate=1:0 to 0:1) to give the title compound (213-6) (1.35 g, 2.32 mmol, 7.11% yield, 73.4% purity) as a yellow oil. LCMS (ES⁺): m/z 428.3 [M+H]⁺.

Synthesis of trans-4-[[5-fluoro-4-[6-(2-oxo-1-piperidyl)-2-pyridyl]pyrimidin-2-yl]amino]cyclohexanecarboxylic acid (213-7). To a solution of trans-4-[[5-fluoro-4-[6-(2-oxo-1-piperidyl)-2-pyridyl]pyrimidin-2-yl]amino]cyclohexanecarboxylate (213-6) (100 mg, 233 μmol, 1.00 eq) in THE (3 mL) and H₂O (1 mL) was added LiOH (16.8 mg, 701 μmol, 3.00 eq). The mixture was stirred at 25 °C for 1 h. TLC indicated trans-4-[[5-fluoro-4-[6-(2-oxo-1-piperidyl)-2-pyridyl]pyrimidin-2-yl]amino]cyclohexanecarboxylate (213-6) remained and one new spot formed. The reaction was clean according to TLC. The reaction mixture was concentrated under high vacuum. The reaction mixture was extracted with EtOAc (7 mL*3), then the water phase was added AcOH (0.05 ml) until pH=5. The water phase was concentrated under high vacuum to give the title compound (213-7) (100 mg, crude) as an orange solid. LCMS (ES⁺): m/z 414.3 [M+H]⁺.

Synthesis of trans-4-((5-fluoro-4-(6-(2-oxopiperidin-1-yl)pyridin-2-yl)pyrimidin-2-yl)amino)cyclohexane-1-carboxamide (Compound 274). To a solution of trans-4-[[5-fluoro-4-[6-(2-oxo-1-piperidyl)-2-pyridyl]pyrimidin-2-yl]amino]cyclohexanecarboxylic acid (213-7) (100 mg, 241 μmol, 1.00 eq) in DMF (3 mL) was added DIEA (62.5 mg, 483 μmol, 84.2 μL, 2.00 eq) until pH-7. Then NH₄Cl (51.7 mg, 967 μmol, 4.00 eq) and HATU (137 mg, 362 μmol, 1.50 eq) was added at 25 °C. The mixture was stirred at 25 °C for 2 h. LC-MS showed the desired mass was detected. The reaction mixture was filtered to give a residue. The residue was purified by prep-HPLC (column: Phenomenex Luna C18 100*30 mm*3 um; mobile phase: [H₂O (0.2% FA)-ACN]; gradient: 10%-40% B over 8.0 min) to give the title compound (Compound 274) (27.0 mg, 65.4 μmol, 25.7% yield, 95.3% purity) as a yellow solid. LCMS (ES⁺): m/z 413.2 [M+H]⁺.

### Example 214. Synthesis of N-(trans-4-((5-fluoro-4-(6-(2-oxopiperidin-1-yl) pyridin-2-yl) pyrimidin-2-yl) amino) cyclohexyl) acetamide (Compound 625)

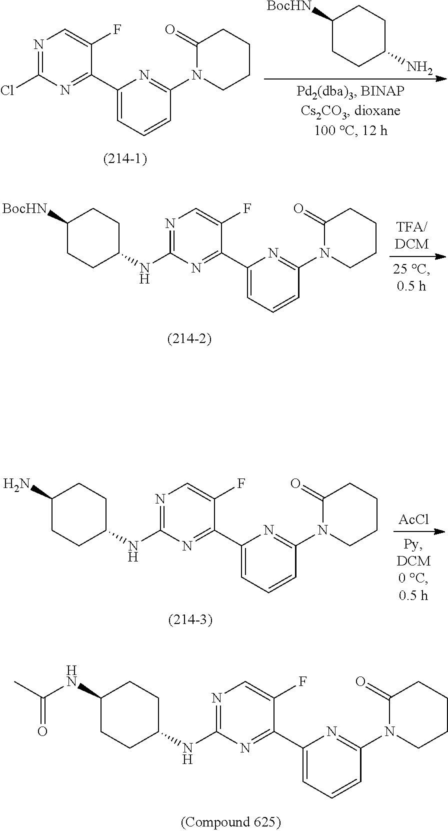

Synthesis of trans-tert-butyl (4-((5-fluoro-4-(6-(2-oxopiperidin-1-yl)pyridin-2-yl)pyrimidin-2-yl)amino)cyclohexyl)carbamate (214-2). To a solution of 1-(6-(2-chloro-5-fluoropyrimidin-4-yl)pyridin-2-yl)piperidin-2-one (214-1) (3.00 g, 9.78 mmol, 1.00 eq) and trans-tert-butyl (4-aminocyclohexyl)carbamate (4.19 g, 19.6 mmol, 2.00 eq) in dioxane (100 mL) were added BINAP (609 mg, 978 μmol, 0.100 eq), Cs₂CO₃ (6.37 g, 19.6 mmol, 2.00 eq) and Pd₂(dba)₃ (895.65 mg, 978.08 μmol, 0.100 eq) at 20 °C under N₂. The mixture was stirred at 100 °C for 12 h under N₂. LCMS showed 1-(6-(2-chloro-5-fluoropyrimidin-4-yl)pyridin-2-yl)piperidin-2-one (214-1) was consumed completely and the desired mass was detected. H₂O (100 mL) was added to the mixture, then the mixture was extracted with EtOAc (50 mL*3). The combined organic layers were dried over Na₂SO₄, filtered and the filtrate was concentrated under reduced pressure to give a residue. The residue was purified by MPLC (SiO₂, PE/EtOAc=1/0 to 1/1) to give the title compound (214-2) (1.64 g, 3.38 mmol, 34.6% yield) as a yellow solid. LCMS (ES⁺): m/z 485.2 [M+H]⁺

Synthesis of trans-1-(6-(2-((4-aminocyclohexyl)amino)-5-fluoropyrimidin-4-yl)pyridin-2-yl)piperidin-2-one (214-3). A solution of trans-tert-butyl (4-((5-fluoro-4-(6-(2-oxopiperidin-1-yl)pyridin-2-yl)pyrimidin-2-yl)amino)cyclohexyl)carbamate (214-2) (210 mg, 433 μmol, 1.00 eq) in DCM (1 mL) and TFA (1 mL) was stirred at 25 °C for 0.5 h. LCMS showed trans-tert-butyl (4-((5-fluoro-4-(6-(2-oxopiperidin-1-yl)pyridin-2-yl)pyrimidin-2-yl)amino)cyclohexyl)carbamate (214-2) was consumed completely and the desired mass was detected. The mixture was concentrated under reduced pressure to give the title compound (214-3) (216 mg, crude, TFA) as a yellow oil. LCMS (ES⁺): m/z 385.2 [M+H]⁺

Synthesis of N-(trans-4-((5-fluoro-4-(6-(2-oxopiperidin-1-yl) pyridin-2-yl) pyrimidin-2-yl) amino) cyclohexyl) acetamide (Compound 625). To a solution of 1-(6-(2-((trans-4-aminocyclohexyl) amino)-5-fluoropyrimidin-4-yl) pyridin-2-yl) piperidin-2-one (214-3) (300 mg, 457 μmol, 1.00 eq, TFA) in DCM (4 mL) was added Py (36.2 mg, 457 mol, 36.9 μL, 1.00 eq) and acetyl chloride (35.9 mg, 457 μmol, 32.5 μL, 1.00 eq) at 0 °C. The resulting mixture was stirred at 0 °C for 0.5 h. LCMS showed that 1-(6-(2-((trans-4-aminocyclohexyl) amino)-5-fluoropyrimidin-4-yl) pyridin-2-yl) piperidin-2-one (214-3) was consumed completely and the desired mass was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 20%-50% B over 8.0 min) to give the title compound (Compound 625) (46.7 mg, 109 μmol, 23.7% yield, 99.2% purity) as a yellow solid. LCMS (ES⁺): m/z 427.2 [M+H]⁺.

### Example 215. Synthesis of trans-N-(4-((5-fluoro-4-(2-(3-oxomorpholino)pyridin-4-yl)pyrimidin-2-yl)amino)cyclohexyl)acetamide (Compound 272)

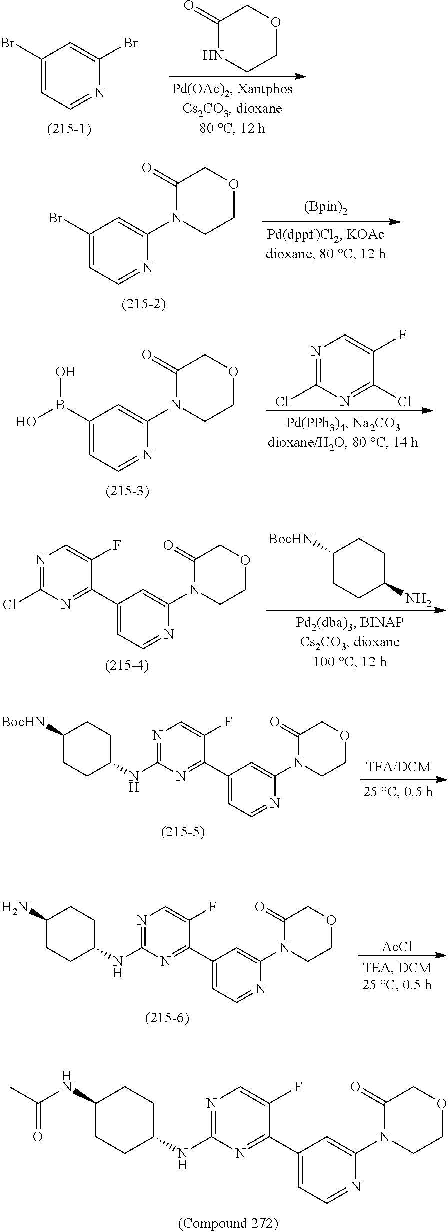

Synthesis of 4-(4-bromopyridin-2-yl)morpholin-3-one (215-2). To a solution of morpholin-3-one (4.29 g, 42.4 mmol, 0.670 eq) in dioxane (380 mL) were added 2,4-dibromopyridine (215-1) (15.0 g, 63.3 mmol, 1.00 eq), Cs₂CO₃ (27.4 g, 84.2 mmol, 1.33 eq), Xantphos (7.33 g, 12.7 mmol, 0.200 eq) and Pd(OAc)₂ (711 mg, 3.17 mmol, 0.0500 eq) at 25 °C under N₂ atmosphere. The mixture was stirred at 80 °C for 12 h under N₂. LC-MS showed 2,4-dibromopyridine (215-1) was consumed completely and the desired mass was detected. TLC (SiO₂, Petroleum ether:Ethyl acetate=1:1) showed 2,4-dibromopyridine (215-1) was consumed, and three new spots formed. Water (350 mL) was added to the mixture at 25 °C. The reaction mixture was extracted with EtOAc (200 mL*5). The organic layers were dried over anhydrous sodium sulfate, filtered and the filtrate was concentrated under reduced to give a residue. The residue was purified by column chromatography (SiO₂, Petroleum ether:Ethyl acetate=4:1 to 1:1) to give the title compound (215-2) (13.9 g, 54.1 mmol, 42.7% yield) as a light yellow solid. LCMS (ES⁺): m/z 257.0.

Synthesis of (2-(3-oxomorpholino)pyridin-4-yl)boronic acid (215-3). To a solution of 4-(4-bromopyridin-2-yl)morpholin-3-one (215-2) (12.9 g, 45.0 mmol, 1.00 eq) in dioxane (500 mL) were added 4,4,5,5-tetramethyl-2-(4,4,5-trimethyl-1,3,2-dioxaborolan-2-yl)-1,3,2-dioxaborolane (36.0 g, 150 mmol, 3.00 eq), KOAc (19.2 g, 200 mmol, 4.00 eq) and Pd(dppf)Cl₂ (3.66 g, 5.00 mmol, 0.100 eq) at 25 °C under N₂ atmosphere. The mixture was stirred at 80 °C for 12 h under N₂. LC-MS showed 4-(4-bromopyridin-2-yl)morpholin-3-one (215-2) was consumed completely and the desired mass was detected. The reaction mixture was concentrated under reduced pressure to give the title compound (215-3) (11.1 g, crude) as a brown solid. It was used directly without further purification. LCMS (ES⁺): m/z 223.2 [M+H]⁺.

Synthesis of 4-(4-(2-chloro-5-fluoropyrimidin-4-yl)pyridin-2-yl)morpholin-3-one (215-4). To a solution of (2-(3-oxomorpholino)pyridin-4-yl)boronic acid (215-3) (11.1 g, 50.0 mmol, 1.00 eq) in dioxane (500 mL) and H₂O (50 mL) were added 2,4-dichloro-5-fluoropyrimidine (20.9 g, 125 mmol, 2.50 eq), Na₂CO₃ (10.6 g, 100 mmol, 2.00 eq) and Pd(PPh₃)₄ (1.16 g, 100 μmol, 0.0200 eq) at 25 °C under N₂ atmosphere. The mixture was stirred at 80 °C for 14 h under N₂. LC-MS showed (2-(3-oxomorpholino)pyridin-4-yl)boronic acid (215-3) was consumed completely, and the desired mass was detected. Water (400 mL) was added to the mixture at 25 °C. The reaction mixture was extracted with EtOAc (300 mL*5). The organic layers were dried over anhydrous sodium sulfate, filtered and the filtrate was concentrated under reduced to give a residue. The residue was purified by column chromatography (SiO₂, Petroleum ether:Ethyl acetate=2:1 to 1:1) to give the title compound (215-4) (15.6 g, crude) as a brown solid. The amount exceeded the theoretical yield, and the purity was 70%, so even though it was purified by prep-HPLC, it was still a crude product. LCMS (ES⁺): m/z 309.0 [M+H]⁺.

Synthesis of trans-tert-butyl (4-((5-fluoro-4-(2-(3-oxomorpholino)pyridin-4-yl)pyrimidin-2-yl)amino)cyclohexyl)carbamate (215-5). To a solution of trans-tert-butyl ((1r,4r)-4-aminocyclohexyl)carbamate (694 mg, 3.24 mmol, 1.00 eq) in dioxane (15 mL) were added 4-(4-(2-chloro-5-fluoropyrimidin-4-yl)pyridin-2-yl)morpholin-3-one (215-4) (1.00 g, 3.24 mmol, 1.00 eq), Cs₂CO₃ (3.17 g, 9.72 mmol, 3.00 eq), BINAP (202 mg, 324 mol, 0.100 eq) and Pd₂(dba)₃ (297 mg, 324 μmol, 0.100 eq) at 25 °C under N₂ atmosphere. The mixture was stirred at 100 °C for 12 h under N₂. LC-MS showed 4-(4-(2-chloro-5-fluoropyrimidin-4-yl)pyridin-2-yl)morpholin-3-one (215-4) was consumed completely and the desired mass was detected. Water (10 mL) was added to the mixture at 25 °C. The reaction mixture was extracted with EtOAc (5 mL*5). The combined organic layers were dried over anhydrous sodium sulfate, filtered and the filtrate was concentrated under reduced to give a residue. The residue was purified by column chromatography (SiO₂, Petroleum ether:Ethyl acetate=2:1 to 1:1) to give the title compound (215-5) (345 mg, 709 μmol, 21.9% yield) as a yellow solid. LCMS (ES⁺): m/z 487.2 [M+H]⁺

Synthesis of trans-4-(4-(2-((4-aminocyclohexyl)amino)-5-fluoropyrimidin-4-yl)pyridin-2-yl)morpholin-3-one (215-6). To a solution of trans-tert-butyl (4-((5-fluoro-4-(2-(3-oxomorpholino)pyridin-4-yl)pyrimidin-2-yl)amino)cyclohexyl)carbamate (215-5) (200 mg, 411 μmol, 1.00 eq) in DCM (3 mL) was added TFA (1.54 g, 13.5 mmol, 1.00 mL, 32.8 eq) at 25 °C. The mixture was stirred at 25 °C for 0.5 h. LC-MS showed trans-tert-butyl (4-((5-fluoro-4-(2-(3-oxomorpholino)pyridin-4-yl)pyrimidin-2-yl)amino)cyclohexyl)carbamate (215-5) was consumed completely and the desired mass was detected. The reaction mixture was concentrated under reduced pressure to give the title compound (215-6) (206 mg, crude, TFA) as a yellow oil. LCMS (ES⁺): m/z 387.1 [M+H]⁺.

Synthesis of trans-N-(4-((5-fluoro-4-(2-(3-oxomorpholino)pyridin-4-yl)pyrimidin-2-yl)amino)cyclohexyl)acetamide (Compound 272). To a solution of trans-4-(4-(2-((4-aminocyclohexyl)amino)-5-fluoropyrimidin-4-yl)pyridin-2-yl)morpholin-3-one (215-6) (206 mg, 532 μmol, 1.00 eq) in DCM (3 mL) was added TEA (162 mg, 1.60 mmol, 222 μL, 3.00 eq) at 25 °C. To the mixture was added acetyl chloride (33.4 mg, 426 μmol, 30.3 μL, 0.800 eq) at 0 °C. The mixture was stirred at 25 °C for 0.5 h. LC-MS showed trans-4-(4-(2-((4-aminocyclohexyl)amino)-5-fluoropyrimidin-4-yl)pyridin-2-yl)morpholin-3-one (215-6) was consumed completely and the desired mass was detected. Water (0.2 mL) was added to the mixture at 25 °C to quench AcCl. The reaction mixture was concentrated under reduced pressure to give a residue. The residue was purified by prep-HPLC (column: 3_Phenomenex Luna C18 75*30 mm*3 um; mobile phase: [H₂O (0.2% FA)-ACN]; gradient: 10%-40% B over 8.0 min) to give the title compound (Compound 272) (82.8 mg, 192 μmol, 36.0% yield, 99.1% purity) as a white solid. LCMS (ES⁺): m/z 429.2 [M+H]⁺.

### Example 216. Synthesis of trans-4-((5-fluoro-4-(2-(3-oxomorpholino)pyridin-4-yl)pyrimidin-2-yl)amino)cyclohexane-1-carboxamide (Compound 273)

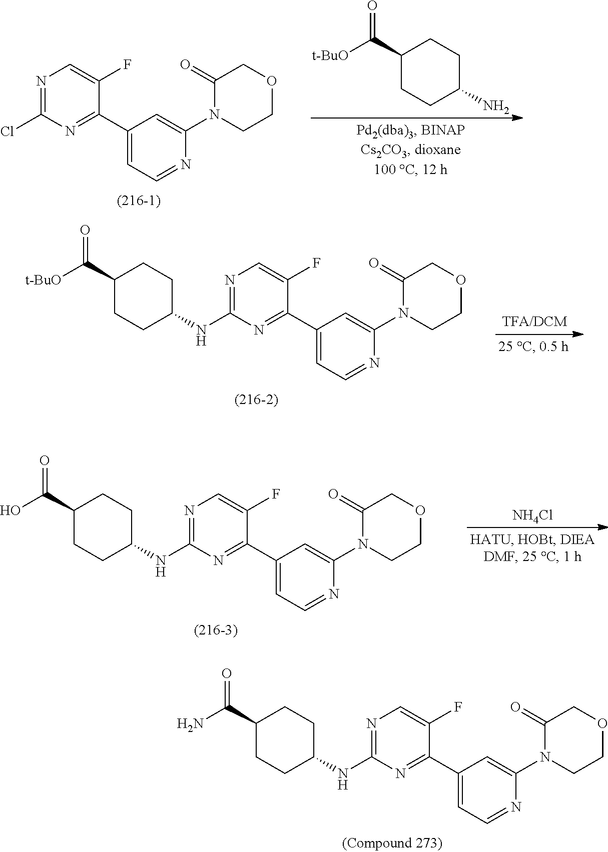 [SMILES: O=C1COCCN1c1cc(-c2nc(Cl)ncc2F)ccn1 ; CC(C)(C)OC(=O)[C@H]1CC[C@H](N)CC1 ; CC(C)(C)OC(=O)[C@H]1CC[C@H](Nc2ncc(F)c(-c3ccnc(N4CCOCC4=O)c3)n2)CC1 ; O=C1COCCN1c1cc(-c2nc(N[C@H]3CC[C@H](C(=O)O)CC3)ncc2F)ccn1 ; "NH4Cl" ; NC(=O)[C@H]1CC[C@H](Nc2ncc(F)c(-c3ccnc(N4CCOCC4=O)c3)n2)CC1]

Synthesis of trans-tert-butyl-4-((5-fluoro-4-(2-(3-oxomorpholino)pyridin-4-yl)pyrimidin-2-yl)amino)cyclohexane-1-carboxylate (216-2). To a solution of trans-tert-butyl-4-aminocyclohexane-1-carboxylate (646 mg, 3.24 mmol, 1.00 eq) in dioxane (15 mL) were added 4-(4-(2-chloro-5-fluoropyrimidin-4-yl)pyridin-2-yl)morpholin-3-one (216-1) (1.00 g, 3.24 mmol, 1.00 eq), Cs₂CO₃ (3.17 g, 9.72 mmol, 3.00 eq), BINAP (202 mg, 324 μmol, 0.100 eq) and Pd₂(dba)₃ (297 mg, 324 μmol, 0.100 eq) at 25 °C under N₂ atmosphere. The mixture was stirred at 100 °C for 12 h under N₂. LC-MS showed 4-(4-(2-chloro-5-fluoropyrimidin-4-yl)pyridin-2-yl)morpholin-3-one (216-1) was consumed completely and the desired mass was detected. Water (10 mL) was added to the mixture at 25 °C. The reaction mixture was extracted with EtOAc (5 mL*5). The organic layers were dried over anhydrous sodium sulfate, filtered and the filtrate was concentrated under reduced to give a residue. The residue was purified by column chromatography (SiO₂, Petroleum ether:Ethyl acetate=2:1 to 1:1) to give the title compound (216-2) (267 mg, 566 μmol, 17.5% yield) as a yellow solid. LCMS (ES⁺): m/z 472.3 [M+H]⁺.

Synthesis of trans-4-((5-fluoro-4-(2-(3-oxomorpholino)pyridin-4-yl)pyrimidin-2-yl)amino)cyclohexane-1-carboxylic acid (216-3). To a solution of trans-tert-butyl-4-((5-fluoro-4-(2-(3-oxomorpholino)pyridin-4-yl)pyrimidin-2-yl)amino)cyclohexane-1-carboxylate (216-2) (260 mg, 551 μmol, 1.00 eq) in DCM (5 mL) was added TFA (1.54 g, 13.5 mmol, 1.00 mL, 24.4 eq) at 25 °C. The mixture was stirred at 25 °C for 0.5 h. LC-MS showed ~16.7% of trans-tert-butyl-4-((5-fluoro-4-(2-(3-oxomorpholino)pyridin-4-yl)pyrimidin-2-yl)amino)cyclohexane-1-carboxylate (216-2) was remaining, and ~70.8% of the desired mass was detected. The mixture was stirred at 25 °C for another 2 h. LC-MS showed trans-tert-butyl-4-((5-fluoro-4-(2-(3-oxomorpholino)pyridin-4-yl)pyrimidin-2-yl)amino)cyclohexane-1-carboxylate (216-2) was consumed completely, and the desired mass was detected. The reaction mixture was concentrated under reduced pressure to give the title compound (216-3) (292 mg, crude, TFA) as a yellow oil. LCMS (ES⁺): m/z 416.1 [M+H]⁺

Synthesis of trans-4-((5-fluoro-4-(2-(3-oxomorpholino)pyridin-4-yl)pyrimidin-2-yl)amino)cyclohexane-1-carboxamide (Compound 273). To a solution of trans-4-((5-fluoro-4-(2-(3-oxomorpholino)pyridin-4-yl)pyrimidin-2-yl)amino)cyclohexane-1-carboxylic acid (216-3) (292 mg, 551 μmol, 1.00 eq, TFA) in DMF (2 mL) were added HATU (252 mg, 662 mol, 1.20 eq), HOBt (37.3 mg, 276 μmol, 0.500 eq), DIEA (356 mg, 2.76 mmol, 480 μL, 5.00 eq) and NH₄Cl (118 mg, 2.21 mmol, 4.00 eq) at 25 °C. The mixture was stirred at 25 °C for 1 h. LC-MS showed trans-4-((5-fluoro-4-(2-(3-oxomorpholino)pyridin-4-yl)pyrimidin-2-yl)amino)cyclohexane-1-carboxylic acid (216-3) was consumed completely and the desired mass was detected. The reaction mixture was concentrated under reduced pressure to give a residue. The residue was purified by prep-HPLC (column: 3_Phenomenex Luna C18 75*30 mm*3 um; mobile phase: [H₂O (0.2% FA)-ACN]; gradient: 10%-40% B over 8.0 min) to give the title compound (Compound 273) (131 mg, 309 μmol, 56.0% yield, 97.8% purity) as a pale yellow solid. LCMS (ES⁺): m/z 415.2 [M+H]⁺.

### Example 217. Synthesis of trans-N-(4-((5-fluoro-4-(6-(3-oxomorpholino)pyridin-2-yl)pyrimidin-2-yl)amino)cyclohexyl)acetamide (Compound 270)

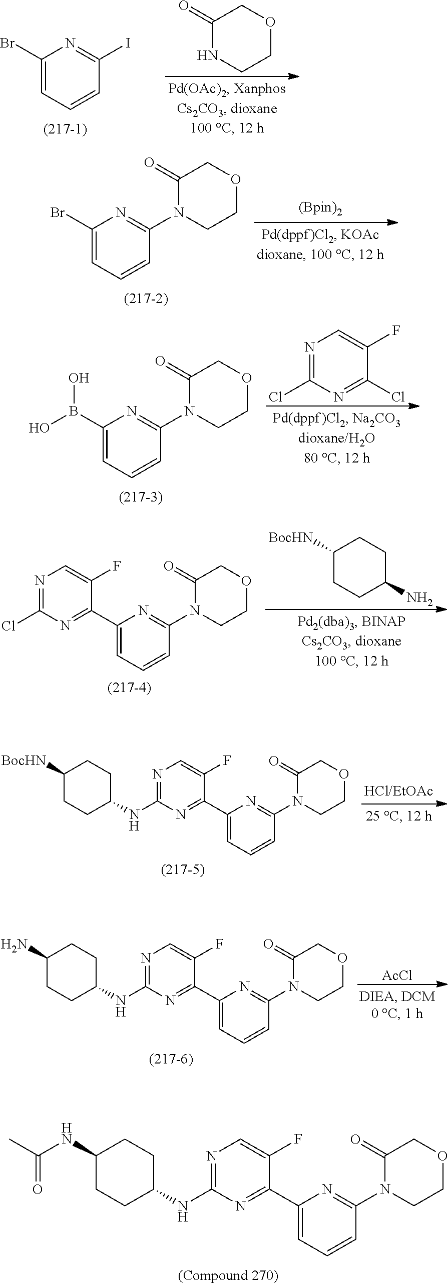

Synthesis of 4-(6-bromopyridin-2-yl)morpholin-3-one (217-2). To a solution of 2-bromo-6-iodopyridine (217-1) (7.00 g, 69.24 mmol, 1.00 eq) and morpholin-3-one (29.5 g, 104 mmol, 1.50 eq) in dioxane (150 mL) were added Pd(OAc)₂ (1.55 g, 6.92 mmol, 0.100 eq), Xantphos (4.01 g, 6.92 mmol, 0.100 eq) and Cs₂CO₃ (67.7 g, 208 mmol, 3.00 eq) at 25 °C under N₂, the reaction was stirred at 100 °C under N₂ for 12 h. LCMS showed 2-bromo-6-iodopyridine (217-1) was consumed completely and the desired mass was detected. The mixture was diluted with H₂O (150 mL) and then extracted with EtOAc (150 mL*3). The combined organic layers were dried over Na₂SO₄, filtered and the filtrate was concentrated under reduced pressure to give a residue. The residue was purified by MPLC (SiO₂, PE/EtOAc=1/0 to 1/1) to give the title compound (217-2) (16.0 g, 62.2 mmol, 89.9% yield) as a yellow solid. LCMS (ES⁺): m/z 257.0 [M+H]⁺.

Synthesis of (6-(3-oxomorpholino)pyridin-2-yl)boronic acid (217-3). To a solution of 4-(6-bromopyridin-2-yl)morpholin-3-one (217-2) (13.3 g, 51.7 mmol, 1.00 eq) and 4,4,5,5-tetramethyl-2-(4,4,5,5-tetramethyl-1,3,2-dioxaborolan-2-yl)-1,3,2-dioxaborolane (26.3 g, 103 mmol, 2.00 eq) in dioxane (220 mL) were added KOAc (10.2 g, 103 mmol, 2.00 eq) and Pd(dppf)Cl₂ (3.79 g, 5.17 mmol, 0.100 eq) under N₂ at 25 °C, the mixture was stirred at 100 °C for 12 h under N₂. LCMS showed 4-(6-bromopyridin-2-yl)morpholin-3-one (217-2) was consumed completely and the desired mass was detected. The mixture was used to next step directly without further work up or purification to give the title compound (217-3) (11.49 g, crude) in dioxane (220 mL) as black liquid. LCMS (ES⁺): m/z 223.1 [M+H]⁺.

Synthesis of 4-(6-(2-chloro-5-fluoropyrimidin-4-yl)pyridin-2-yl)morpholin-3-one (217-4). To a solution of (6-(3-oxomorpholino)pyridin-2-yl)boronic acid (217-3) (750 mg, 2.47 mmol, 1.00 eq) and 2,4-dichloro-5-fluoropyrimidine (618 mg, 3.70 mmol, 1.50 eq) in dioxane (14.4 mL) and H₂O (1.44 mL) were added Pd(dppf)Cl₂ (180 mg, 246.59 μmol, 0.100 eq) and Na₂CO₃ (784 mg, 7.40 mmol, 3.00 eq) under N₂ at 25 C, the mixture was stirred at 80 °C for 12 h under N₂. LCMS showed (6-(3-oxomorpholino)pyridin-2-yl)boronic acid (217-3) was consumed completely and the desired mass was detected. The mixture was diluted with H₂O (15 mL) and then extracted with EtOAc (15 mL*3). The combined organic layers were dried over Na₂SO₄, filtered and the filtrate was concentrated under reduced pressure to give a residue. The residue was purified by MPLC (SiO₂, PE/EtOAc=1/0 to 3/1) to give the title compound (217-4) (470 mg, 1.52 mmol, 61.7% yield) as a yellow solid. LCMS (ES⁺): m/z 309.0 [M+H]⁺.

Synthesis of trans-tert-butyl (4-((5-fluoro-4-(6-(3-oxomorpholino)pyridin-2-yl)pyrimidin-2-yl)amino)cyclohexyl)carbamate (217-5). To a solution of 4-(6-(2-chloro-5-fluoropyrimidin-4-yl)pyridin-2-yl)morpholin-3-one (217-4) (460 mg, 1.49 mmol, 1.00 eq) and trans-tert-butyl (4-aminocyclohexyl)carbamate (639 mg, 2.98 mmol, 2.00 eq) in dioxane (10 mL) were added Pd₂(dba)₃ (136 mg, 149 μmol, 0.100 eq), BINAP (92.8 mg, 149.01 mol, 0.100 eq) and Cs₂CO₃ (1.46 g, 4.47 mmol, 3.00 eq) at 25 °C under N₂, the mixture was stirred at 100 °C under N₂ for 12 h. LCMS showed 4-(6-(2-chloro-5-fluoropyrimidin-4-yl)pyridin-2-yl)morpholin-3-one (217-4) was consumed completely and the desired mass was detected. The mixture was diluted with H₂O (10 mL) and then extracted with EtOAc (10 mL*3). The combined organic layers were dried over Na₂SO₄, filtered and the filtrate was concentrated under reduced pressure to give a residue. The residue was purified by prep-TLC (SiO₂, PE/EtOAc=0/1) to give the title compound (217-5) (300 mg, 617 μmol, 41.4% yield) as a white solid. LCMS (ES⁺): m/z 487.2 [M+H]⁺.

Synthesis of trans-4-(6-(2-((4-aminocyclohexyl)amino)-5-fluoropyrimidin-4-yl)pyridin-2-yl)morpholin-3-one (217-6). A solution of trans-tert-butyl (4-((5-fluoro-4-(6-(3-oxomorpholino)pyridin-2-yl)pyrimidin-2-yl)amino)cyclohexyl)carbamate (217-5) (300 mg, 617 μmol, 1.00 eq) in HCl/EtOAc (4 mL) was stirred at 25 °C for 12 h. LCMS showed trans-tert-butyl (4-((5-fluoro-4-(6-(3-oxomorpholino)pyridin-2-yl)pyrimidin-2-yl)amino)cyclohexyl)carbamate (217-5) was consumed completely and the desired mass was detected. The mixture was concentrated under reduced pressure to give the title compound (217-6) (300 mg, crude, HCl) as a yellow oil. LCMS (ES⁺): m/z 387.1 [M+H]⁺.

Synthesis of trans-N-(4-((5-fluoro-4-(6-(3-oxomorpholino)pyridin-2-yl)pyrimidin-2-yl)amino)cyclohexyl)acetamide (Compound 270). To a solution of trans-4-(6-(2-((4-aminocyclohexyl)amino)-5-fluoropyrimidin-4-yl)pyridin-2-yl)morpholin-3-one (217-6) (300 mg, 709 μmol, 1.00 eq, HCl) in DCM (4 mL) was added DIEA (275 mg, 2.13 mmol, 371 μL, 3.00 eq) at 25 °C, to the reaction was added acetyl chloride (44.6 mg, 568 μmol, 40.4 μL, 0.800 eq) at 0 °C, The resulting mixture was stirred at 0 °C for 1 h. LCMS showed trans-4-(6-(2-((4-aminocyclohexyl)amino)-5-fluoropyrimidin-4-yl)pyridin-2-yl)morpholin-3-one (217-6) was consumed completely and the desired mass was detected. Water (0.5 mL) was added to the mixture to quench AcCl. Then it was concentrated under reduced pressure to give a residue. The residue was purified by prep-HPLC (column: Phenomenex luna C18 100*40 mm*5 um; mobile phase: [H₂O (0.2% FA)-ACN]; gradient: 15%-55% B over 8.0 min) to give the title compound (Compound 270) (15.7 mg, 35.6 μmol, 5.02% yield, 97.2% purity) as a yellow solid. LCMS (ES⁺): m/z 429.1 [M+H]⁺.

### Example 218. Synthesis of trans-4-((5-fluoro-4-(6-(3-oxomorpholino)pyridin-2-yl)pyrimidin-2-yl)amino)cyclohexane-1-carboxamide (Compound 271)

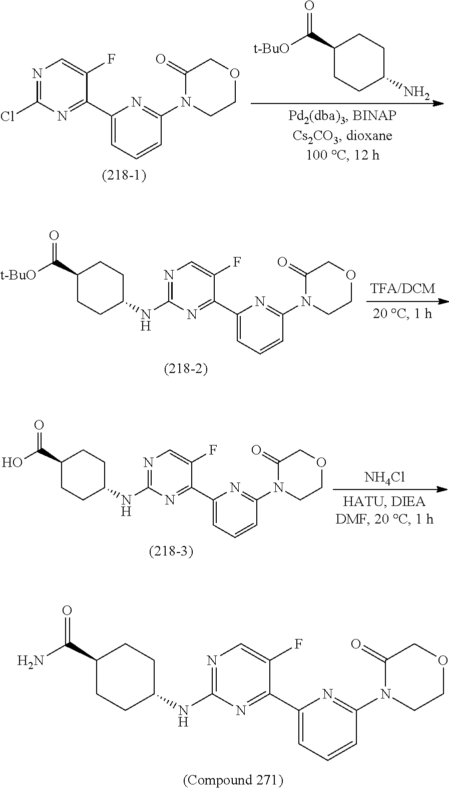 [SMILES: O=C1COCCN1c1cccc(-c2nc(Cl)ncc2F)n1 ; CC(C)(C)OC(=O)[C@H]1CC[C@H](N)CC1 ; CC(C)(C)OC(=O)[C@H]1CC[C@H](Nc2ncc(F)c(-c3cccc(N4CCOCC4=O)n3)n2)CC1 ; O=C1COCCN1c1cccc(-c2nc(N[C@H]3CC[C@H](C(=O)O)CC3)ncc2F)n1 ; "NH4Cl" ; NC(=O)[C@H]1CC[C@H](Nc2ncc(F)c(-c3cccc(N4CCOCC4=O)n3)n2)CC1]

Synthesis of trans-tert-butyl-4-((5-fluoro-4-(6-(3-oxomorpholino)pyridin-2-yl)pyrimidin-2-yl)amino)cyclohexane-1-carboxylate (218-2). To a stirred solution of 4-(6-(2-chloro-5-fluoropyrimidin-4-yl)pyridin-2-yl)morpholin-3-one (218-1) (500 mg, 1.62 mmol, 1.00 eq) and trans-tert-butyl-4-aminocyclohexane-1-carboxylate (646 mg, 3.24 mmol, 2.00 eq) in dioxane (10 mL) were added Cs₂CO₃ (1.06 g, 3.24 mmol, 2.00 eq), BINAP (50.4 mg, 81.0 μmol, 0.0500 eq) and Pd₂(dba)₃ (74.2 mg, 81.0 μmol, 0.0500 eq) at 20 °C under N₂. The resulting mixture was stirred at 100 °C for 12 h under N₂. LCMS showed 4-(6-(2-chloro-5-fluoropyrimidin-4-yl)pyridin-2-yl)morpholin-3-one (218-1) was consumed completely and the desired mass was detected. The mixture was diluted with H₂O (10 mL), then extracted with EtOAc (10 mL*3). The combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (218-2) (400 mg, 848 μmol, 52.4% yield) as a yellow solid. LCMS (ES⁺): m/z 472.2 [M+H]⁺.

Synthesis of trans-4-((5-fluoro-4-(6-(3-oxomorpholino)pyridin-2-yl)pyrimidin-2-yl)amino)cyclohexane-1-carboxylic acid (218-3). A solution of trans-tert-butyl-4-((5-fluoro-4-(6-(3-oxomorpholino)pyridin-2-yl)pyrimidin-2-yl)amino)cyclohexane-1-carboxylate (218-2) (350 mg, 742 μmol, 1.00 eq) in DCM (4 mL) and TFA (2 mL) was stirred at 20 °C for 1 h. LCMS showed trans-tert-butyl-4-((5-fluoro-4-(6-(3-oxomorpholino)pyridin-2-yl)pyrimidin-2-yl)amino)cyclohexane-1-carboxylate (218-2) was consumed completely and the desired mass was detected. The mixture was concentrated under reduced pressure to give the title compound (218-3) (310 mg, crude) as a yellow oil. LCMS (ES⁺): m/z 416.1 [M+H]⁺.

Synthesis of trans-4-((5-fluoro-4-(6-(3-oxomorpholino)pyridin-2-yl)pyrimidin-2-yl)amino)cyclohexane-1-carboxamide (Compound 271). To a stirred solution of trans-4-((5-fluoro-4-(6-(3-oxomorpholino)pyridin-2-yl)pyrimidin-2-yl)amino)cyclohexane-1-carboxylic acid (218-3) (310 mg, 746 μmol, 1.00 eq) and DIEA (289 mg, 2.24 mmol, 390 μL, 3.00 eq) in DMF (1 mL) were added HATU (426 mg, 1.12 mmol, 1.50 eq) and NH₄Cl (79.8 mg, 1.49 mmol, 2.00 eq) at 20 °C. The resulting mixture was stirred at 20 °C for 1 h. LCMS showed trans-4-((5-fluoro-4-(6-(3-oxomorpholino)pyridin-2-yl)pyrimidin-2-yl)amino)cyclohexane-1-carboxylic acid (218-3) was consumed completely and the desired mass was detected. The mixture was concentrated under reduced pressure to give a residue. The residue was purified by prep-HPLC (column: Waters Xbridge Prep OBD C18 150*40 mm*Oum; mobile phase: [H₂O (10 mM NH₄HCO₃)-ACN]; gradient: 10%-40% B over 8.0 min) to give the title compound (Compound 271) (35.2 mg, 84.5 μmol, 11.3% yield, 99.5% purity) as a white solid. LCMS (ES⁺): m/z 415.1 [M+H]⁺.

### Example 219. Synthesis of (S)-1-(3-((5-chloro-4-(4′-fluoro-[1,1′-biphenyl]-3-yl)pyrimidin-2-yl)amino)piperidin-1-yl)ethan-1-one (Compound 128)

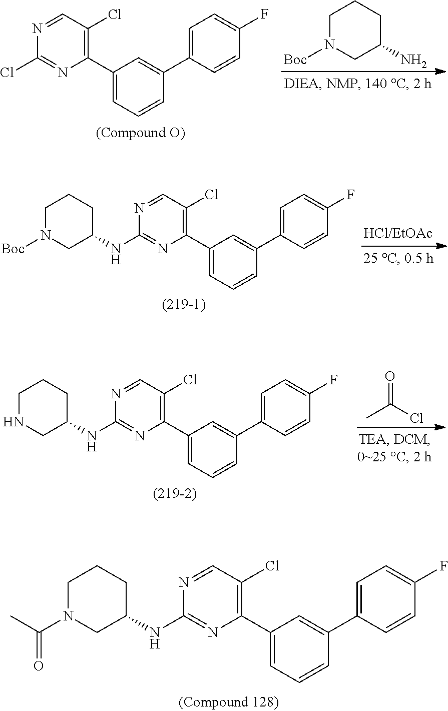

Synthesis of tert-butyl (S)-3-((5-chloro-4-(4′-fluoro-[1,1′-biphenyl]-3-yl)pyrimidin-2-yl)amino)piperidine-1-carboxylate (219-1). To a solution of 2,5-dichloro-4-(4′-fluoro-[1,1′-biphenyl]-3-yl)pyrimidine (Compound O) (200 mg, 626.64 μmol, 1 eq) in NMP (2 mL) was added tert-butyl (3S)-3-aminopiperidine-1-carboxylate (125.50 mg, 626.64 mol, 1 eq) and DIEA (242.97 mg, 1.88 mmol, 327.45 μL, 3 eq). The mixture was stirred at 140 °C for 2 h. LCMS showed 2,5-dichloro-4-(4′-fluoro-[1,1′-biphenyl]-3-yl)pyrimidine (Compound O) was consumed completely and desired mass was detected. The reaction mixture was partitioned between H₂O (20 mL) and EtOAc (20 mL*3). The organic phase was separated, washed with brine (20 mL), dried over Na₂SO₄, filtered and concentrated under reduced pressure to give title compound (219-1) (302.6 mg, crude) as a yellow oil. LCMS (ES+): m/z 427.0 [M+H−56]⁺.

Synthesis of (S)-5-chloro-4-(4′-fluoro-[1,1′-biphenyl]-3-yl)-N-(piperidin-3-yl)pyrimidin-2-amine (219-2). A solution of tert-butyl (S)-3-((5-chloro-4-(4′-fluoro-[1,1′-biphenyl]-3-yl)pyrimidin-2-yl)amino)piperidine-1-carboxylate (219-1) (302 mg, 625.29 μmol, 1 eq) in HCl/EtOAc (5 mL) was stirred at 25 °C for 0.5 h. LCMS showed tert-butyl (S)-3-((5-chloro-4-(4′-fluoro-[1,1′-biphenyl]-3-yl)pyrimidin-2-yl)amino)piperidine-1-carboxylate (219-1) was consumed completely and desired mass was detected. The reaction mixture was concentrated under reduced pressure to remove give title compound (219-2) (191 mg, 498.88 μmol, 79.78% yield) as a white solid. LCMS (ES+): m/z 383.0 [M+H]⁺.

Synthesis of (S)-1-(3-((5-chloro-4-(4′-fluoro-[1,1′-biphenyl]-3-yl)pyrimidin-2-yl)amino)piperidin-1-yl)ethan-1-one (Compound 128). To a solution of (S)-5-chloro-4-(4′-fluoro-[1,1′-biphenyl]-3-yl)-N-(piperidin-3-yl)pyrimidin-2-amine (219-2) (140 mg, 365.67 μmol, 1 eq) and TEA (185.01 mg, 1.83 mmol, 254.48 μL, 5 eq) in DCM (3 mL) was slowly added acetyl chloride (28.70 mg, 365.67 μmol, 26.00 μL, 1 eq) at 0 °C. The reaction mixture was stirred at 25 °C for 2 h. LCMS showed (S)-5-chloro-4-(4′-fluoro-[1,1′-biphenyl]-3-yl)-N-(piperidin-3-yl)pyrimidin-2-amine (219-2) was consumed completely and desired mass was detected. The reaction mixture was concentrated under reduced pressure to remove to give a residue. The residue was purified by prep-HPLC (FA condition; column: Welch Ultimate C18 150*25 mm*5 um; mobile phase: [water (FA)-ACN]; gradient: 55%-85% B over 10 min) to give title compound (Compound 128) (37.6 mg, 85.57 μmol, 23.40% yield, 96.7% purity) as a white solid. LCMS (ES+): m/z 425.0 [M+H]⁺.

### Example 220. Synthesis of (S)-6-(3-(2-((1-acetylpiperidin-3-yl)amino)-5-chloropyrimidin-4-yl)phenyl)pyridin-2(1H)-one (Compound 157)

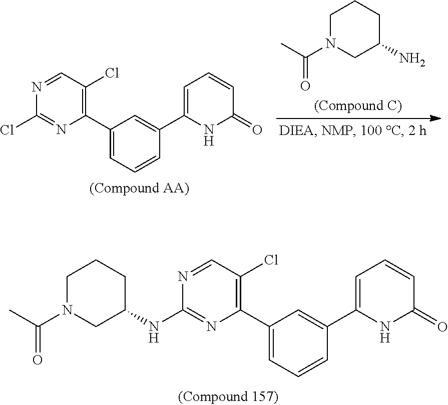

Synthesis of(S)-6-(3-(2-((1-acetylpiperidin-3-yl)amino)-5-chloropyrimidin-4-yl)phenyl)pyridin-2(1H)-one (Compound 157). To a solution of 6-[3-(2,5-dichloropyrimidin-4-yl)phenyl]-1H-pyridin-2-one (Compound AA) (200 mg, 628.62 μmol, 1 eq) in NMP (2 mL) was added (S)-1-(3-aminopiperidin-1-yl)ethan-1-one (Compound C) (89.39 mg, 628.62 μmol, 1 eq), DIEA (406.21 mg, 3.14 mmol, 547.46 μL, 5 eq) under N₂, and the reaction mixture was stirred at 100 °C for 2 h under N₂. LCMS showed 6-[3-(2,5-dichloropyrimidin-4-yl)phenyl]-1H-pyridin-2-one (Compound AA) was consumed completely and desired mass was detected. The reaction mixture was filtered and the filtrate was concentrated to give a residue. The residue was purified by reversed-phase HPLC (column: Phenomenex luna C18 150*25 mm*10 um; mobile phase: [water(FA)-ACN]; gradient: 23%-53% B over 10 min) to give title compound (Compound 157) (65.5 mg, 154.52 μmol, 24.58% yield, 100% purity) as a white solid. LCMS (ES+): m/z 424.0 [M+H]⁺.

### Example 221. Synthesis of (S)-1-(3-((5-chloro-4-(6-(4-fluorophenyl)pyridin-2-yl)pyrimidin-2-yl)amino)piperidin-1-yl)ethan-1-one (Compound 194)

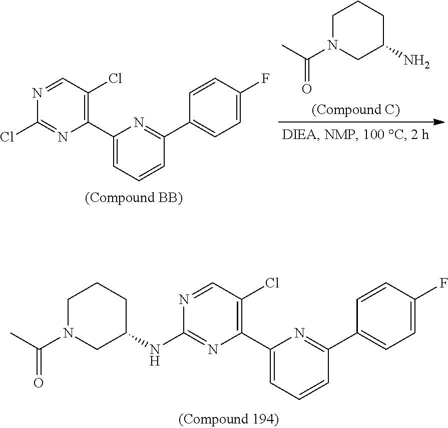

Synthesis of (S)-1-(3-((5-chloro-4-(6-(4-fluorophenyl)pyridin-2-yl)pyrimidin-2-yl)amino)piperidin-1-yl)ethan-1-one (Compound 194). To a solution of 2,5-dichloro-4-[6-(4-fluorophenyl)-2-pyridyl]pyrimidine (Compound BB) (0.1 g, 312.36 μmol, 1 eq) in NMP (3 mL) was added DIEA (201.85 mg, 1.56 mmol, 272.03 μL, 5 eq) and (S)-1-(3-aminopiperidin-1-yl)ethan-1-one (Compound C) (55.81 mg, 312.36 μmol, 1 eq, HCl). The mixture was stirred at 100 °C for 2 h. LC-MS showed 2,5-dichloro-4-[6-(4-fluorophenyl)-2-pyridyl]pyrimidine (Compound BB) was consumed completely and desired mass was detected. The residue was diluted with H₂O (30 mL) and extracted with EtOAc (30 mL*3). The combined organic layers were dried over Na₂SO₄, filtered and the filtrate was concentrated to give a residue. The reaction mixture was dissolved in DMF (5 mL), and the resulting solution was purified by prep-HPLC (neutral condition, column: Waters xbridge 150*25 mm 10 um; mobile phase: [water (NH₄HCO₃)-ACN]; gradient: 46%-76% B over 14 min) to give title compound (Compound 194) (9.5 mg, 21.29 μmol, 6.82% yield, 95.43% purity) as a white solid. LCMS (ES+): m/z 426.1 [M+H]⁺.

### Example 222. Synthesis of (S)-1-(3-((5-chloro-4-(2-(4-fluorophenyl)pyridin-4-yl)pyrimidin-2-yl)amino)piperidin-1-yl)ethan-1-one (Compound 141)

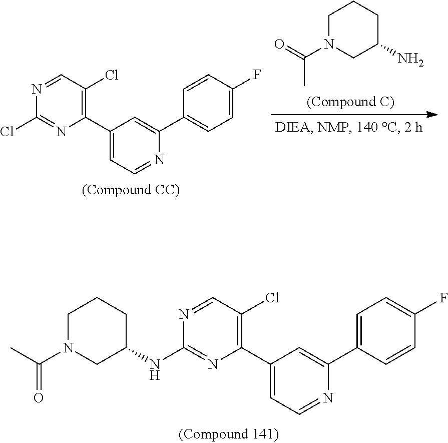

Synthesis of (S)-1-(3-((5-chloro-4-(2-(4-fluorophenyl)pyridin-4-yl)pyrimidin-2-yl)amino)piperidin-1-yl)ethan-1-one (Compound 141). To a solution of 2,5-dichloro-4-[2-(4-fluorophenyl)-4-pyridyl]pyrimidine (Compound CC) (0.1 g, 312.36 μmol, 1 eq) in NMP (2 mL) was added DIEA (201.85 mg, 1.56 mmol, 272.03 μL, 5 eq) and (S)-1-(3-aminopiperidin-1-yl)ethan-1-one (Compound C) (55.81 mg, 312.36 μmol, 1 eq, HCl). The mixture was stirred at 140 °C for 2 h. LCMS showed 2,5-dichloro-4-[2-(4-fluorophenyl)-4-pyridyl]pyrimidine (Compound CC) was consumed completely and desired mass was detected. The reaction did not work up, purified directly. The reaction mixture was dissolved in DMF (5 mL), and the resulting solution was purified by prep-HPLC (FA condition, column: Phenomenex luna C18 150*25 mm*10 um; mobile phase: [water(FA)-ACN]; gradient: 44%-74% B over 10 min) to give title compound (Compound 141) (67.9 mg, 156.74 μmol, 50.18% yield, 98.31% purity) as a white solid. LCMS (ES⁺): m/z 426.0 [M+H]⁺.

### Example 223. Synthesis of (S)-5-(2-((1-acetylpiperidin-3-yl)amino)-5-chloropyrimidin-4-yl)-1-(4-fluorophenyl)pyridin-2(1H)-one (Compound 143)

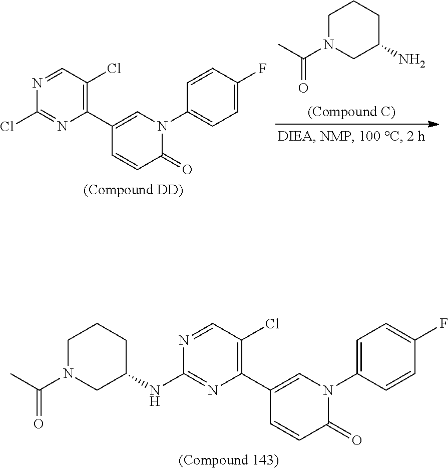

Synthesis of (S)-5-(2-((1-acetylpiperidin-3-yl)amino)-5-chloropyrimidin-4-yl)-1-(4-fluorophenyl)pyridin-2(1H)-one (Compound 143). To a solution of 5-(2,5-dichloropyrimidin-4-yl)-1-(4-fluorophenyl)pyridin-2-one (Compound DD) (0.1 g, 297.49 μmol, 1 eq) in NMP (2 mL) was added DIEA (192.24 mg, 1.49 mmol, 259.09 μL, 5 eq) and (S)-1-(3-aminopiperidin-1-yl)ethan-1-one (Compound C). The mixture was stirred at 140 °C for 2 h. LCMS showed 5-(2,5-dichloropyrimidin-4-yl)-1-(4-fluorophenyl)pyridin-2-one (Compound DD) was consumed completely and desired mass was detected. The reaction mixture was dissolved in DMF (5 mL), and the resulting solution was purified by prep-HPLC (FA condition, column: Phenomenex luna C18 150*25 mm*10 um; mobile phase: [water(FA)-ACN]; gradient: 28%-58% B over 10 min) to give title compound (Compound 143) (37.1 mg, 79.04 μmol, 26.57% yield, 94.14% purity) as a white solid. LCMS (ES⁺): m/z 442.1 [M+H]⁺.

### Example 224. Synthesis of (S)-1-(3-((5-chloro-4-(5-cyclopropyl-1H-pyrazol-3-yl)pyrimidin-2-yl)amino)piperidin-1-yl)ethan-1-one (Compound 192)

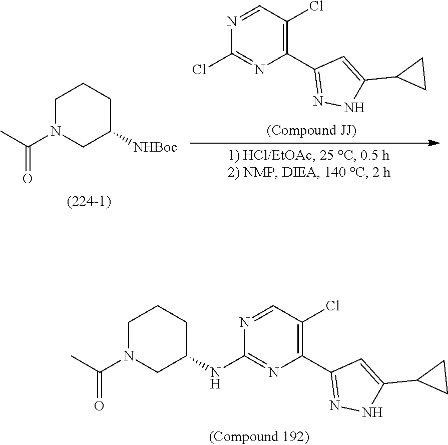 [SMILES: CC(=O)N1CCC[C@H](NC(=O)OC(C)(C)C)C1 ; Clc1ncc(Cl)c(-c2cc(C3CC3)[nH]n2)n1 ; CC(=O)N1CCC[C@H](Nc2ncc(Cl)c(-c3cc(C4CC4)[nH]n3)n2)C1]

Synthesis of (S)-1-(3-((5-chloro-4-(5-cyclopropyl-1H-pyrazol-3-yl)pyrimidin-2-yl)amino)piperidin-1-yl)ethan-1-one (Compound 192) A solution of tert-butyl (S)-(1-acetylpiperidin-3-yl)carbamate (224-1) (83.61 mg, 588.00 μmol, 1 eq) in HCl/EtOAc (3 mL) was stirred at 25 °C for 0.5 h. The reaction mixture was concentrated under reduced pressure to give a residue and then the residue in NMP (3 mL) was added 2,5-dichloro-4-(5-cyclopropyl-1H-pyrazol-3-yl)pyrimidine (Compound JJ) (0.15 g, 588.00 μmol, 1 eq) and DIEA (75.99 mg, 588.00 μmol, 102.42 μL, 1 eq) at 25 °C. The mixture was stirred at 140 °C for 2 h. LCMS showed 2,5-dichloro-4-(5-cyclopropyl-1H-pyrazol-3-yl)pyrimidine (Compound JJ) was consumed completely and the desired mass was detected. The reaction did not work up, purified directly. The residue was purified by prep-HPLC (neutral condition) to give title compound (Compound 192) (0.015 g, 39.74 μmol, 6.76% yield, 95.6% purity) as a white solid. LCMS (ES⁺): m/z 361.2 [M+H]⁺.

### Example 225. Synthesis of 1-(4-((5-chloro-4-(6-(4-fluorophenyl)pyridin-2-yl)pyrimidin-2-yl)amino)piperidin-1-yl)ethan-1-one (Compound 114)

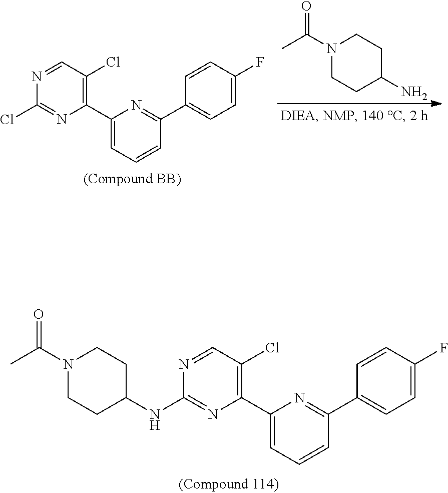

Synthesis of 1-(4-((5-chloro-4-(6-(4-fluorophenyl)pyridin-2-yl)pyrimidin-2-yl)amino)piperidin-1-yl)ethan-1-one (Compound 114). To a solution of 2,5-dichloro-4-[6-(4-fluorophenyl)-2-pyridyl]pyrimidine (Compound BB) (0.1 g, 312.36 μmol, 1 eq) in NMP (3 mL) was added DIEA (201.85 mg, 1.56 mmol, 272.03 μL, 5 eq) and 1-(4-aminopiperidin-1-yl)ethan-1-one (55.81 mg, 312.36 μmol, 1 eq, HCl). The mixture was stirred at 140 °C for 2 h. LCMS showed 2,5-dichloro-4-[6-(4-fluorophenyl)-2-pyridyl]pyrimidine (Compound BB) was consumed completely and desired mass was detected. The residue was diluted with H₂O (30 mL) and extracted with EtOAc (30 mL*3). The combined organic layers were dried over Na₂SO₄, filtered and the filtrate was concentrated to give a residue. The reaction mixture was dissolved in DMF (5 mL), and the resulting solution was purified by prep-HPLC (neutral condition, column: Waters xbridge 150*25 mm 10 um; mobile phase: [water-ACN]; gradient: 46%-76% B over 14 min) to give compound (Compound 114) (25.6 mg, 59.68 μmol, 6.82% yield, 99.28% purity) as a white solid. LCMS (ES+): m/z 426.0 [M+H]⁺.

### Example 226. Synthesis of 1-(4-((5-chloro-4-(2-(4-fluorophenyl)pyridin-4-yl)pyrimidin-2-yl)amino)piperidin-1-yl)ethan-1-one (Compound 105)

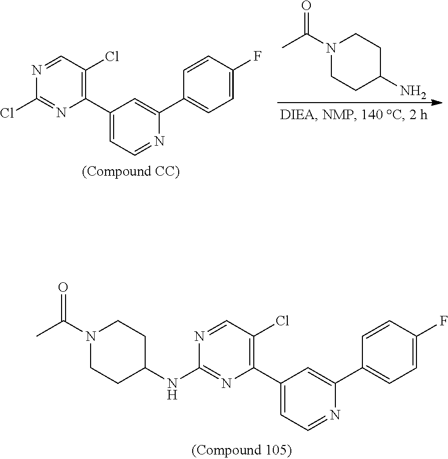

Synthesis of 1-(4-((5-chloro-4-(2-(4-fluorophenyl)pyridin-4-yl)pyrimidin-2-yl)amino)piperidin-1-yl)ethan-1-one (Compound 105). To a solution of 2,5-dichloro-4-[2-(4-fluorophenyl)-4-pyridyl]pyrimidine (Compound CC) (0.1 g, 312.36 μmol, 1 eq) in NMP (2 mL) was added DIEA (201.85 mg, 1.56 mmol, 272.03 μL, 5 eq) and 1-(4-aminopiperidin-1-yl)ethan-1-one (55.81 mg, 312.36 μmol, 1 eq, HCl). The mixture was stirred at 140 °C for 2 h. LCMS showed 2,5-dichloro-4-[2-(4-fluorophenyl)-4-pyridyl]pyrimidine (Compound CC) was consumed completely and desired mass was detected. The reaction did not work up, purified directly. The reaction mixture was dissolved in DMF (5 mL), and the resulting solution was purified by prep-HPLC (FA condition, column: Phenomenex luna C18 150*25 mm*10 um; mobile phase: [water(FA)-ACN]; gradient: 44%-74% B over 10 min) to give title compound (Compound 105) (40.9 mg, 96.25 μmol, 19.77% yield, 97.39% purity) as a white solid. LCMS (ES⁺): m/z 426.0 [M+H]⁺.

### Example 227. Synthesis of 1-(4-((5-chloro-4-(1-cyclopropyl-1H-pyrazol-4-yl)pyrimidin-2-yl)amino)piperidin-1-yl)ethan-1-one (Compound 98)

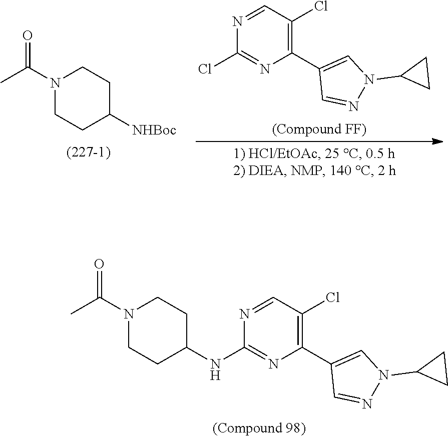 [SMILES: CC(=O)N1CCC(NC(=O)OC(C)(C)C)CC1 ; Clc1ncc(Cl)c(-c2cnn(C3CC3)c2)n1 ; CC(=O)N1CCC(Nc2ncc(Cl)c(-c3cnn(C4CC4)c3)n2)CC1]

Synthesis of 1-(4-((5-chloro-4-(1-cyclopropyl-1H-pyrazol-4-yl)pyrimidin-2-yl)amino)piperidin-1-yl)ethan-1-one (Compound 98) To a solution of tert-butyl (4-acetylcyclohexyl)carbamate (227-1) (150.73 mg, 588.00 μmol, 1 eq) in HCl/EtOAc (2 mL), the mixture was stirred at 25 °C for 0.5 h. The reaction mixture was concentrated under reduced pressure to give a residue and then the residue in NMP (3 mL) was added 2,5-dichloro-4-(1-cyclopropylpyrazol-4-yl)pyrimidine (Compound FF) (150 mg, 588.00 μmol, 1 eq) and DIEA (227.98 mg, 1.76 mmol, 307.26 μL, 3 eq) at 25 °C, the mixture was stirred at 140 °C for 2 h. LCMS showed 2,5-dichloro-4-(1-cyclopropylpyrazol-4-yl)pyrimidine (Compound FF) was consumed completely and the desired mass was detected. The reaction did not work up, purified directly. The residue was purified by prep-HPLC (neutral condition; column:column: Phenomenex C18 150*25 mm*10 um; mobile phase: [water(NH₄HCO₃)-ACN]; gradient: 26%-56% B over 14 min) to give title compound (Compound 98) (0.029 g, 79.00 μmol, 13.44% yield, 98.3% purity) as a white solid. LCMS (ES+): m/z 361.1 [M+H]⁺.

### Example 228. Synthesis of 1-(4-((5-chloro-4-(1-cyclohexyl-1H-pyrazol-3-yl)pyrimidin-2-yl)amino)piperidin-1-yl)ethan-1-one (Compound 109)

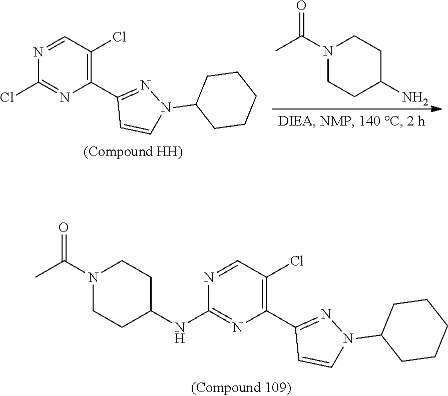

Synthesis of 1-[4-[[5-chloro-4-(1-cyclohexylpyrazol-3-yl)pyrimidin-2-yl]amino]-1-piperidyl]ethanone (Compound 109). To a solution of 2,5-dichloro-4-(1-cyclohexylpyrazol-3-yl)pyrimidine (Compound HH) (0.15 g, 504.74 μmol, 1 eq) in NMP (3 mL) was added 1-(4-amino-1-piperidyl)ethanone (71.77 mg, 504.74 μmol, 1 eq) and DIEA (65.23 mg, 504.74 μmol, 87.91 μL, 1 eq). The mixture was stirred at 140 °C for 2 h. LCMS showed 2,5-dichloro-4-(1-cyclohexylpyrazol-3-yl)pyrimidine (Compound HH) was consumed completely and the desired mass was detected. The reaction did not work up, purified directly. The residue was purified by prep-HPLC (neutral condition) to give title compound (Compound 109) (9 mg, 22.16 μmol, 4.39% yield, 99.2% purity) as a white solid. LCMS (ES⁺): m/z 403.1 [M+H]⁺.

### Example 229. Synthesis of 1-(4-((5-chloro-4-(5-cyclopropyl-1H-pyrazol-3-yl)pyrimidin-2-yl)amino)piperidin-1-yl)ethan-1-one (Compound 132)

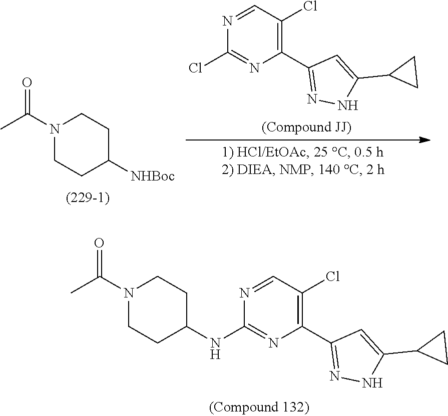 [SMILES: CC(=O)N1CCC(NC(=O)OC(C)(C)C)CC1 ; Clc1ncc(Cl)c(-c2cc(C3CC3)[nH]n2)n1 ; CC(=O)N1CCC(Nc2ncc(Cl)c(-c3cc(C4CC4)[nH]n3)n2)CC1]

Synthesis of 1-(4-((5-chloro-4-(5-cyclopropyl-1H-pyrazol-3-yl)pyrimidin-2-yl)amino)piperidin-1-yl)ethan-1-one (Compound 132). To a solution of tert-butyl (1-acetylpiperidin-4-yl)carbamate (229-1) (83.61 mg, 588.00 μmol, 1 eq) in HCl/EtOAc (3 mL), the mixture was stirred at 25 °C for 0.5 h. The reaction mixture was concentrated under reduced pressure to give a residue and then the residue in NMP (3 mL) was added 2,5-dichloro-4-(5-cyclopropyl-1H-pyrazol-3-yl)pyrimidine (Compound JJ) (0.15 g, 588.00 μmol, 1 eq) and DIEA (75.99 mg, 588.00 μmol, 102.42 μL, 1 eq) at 25 °C the mixture was stirred at 140 °C for 2 h. LCMS showed 2,5-dichloro-4-(5-cyclopropyl-1H-pyrazol-3-yl)pyrimidine (Compound JJ) was consumed completely and the desired mass was detected. The reaction did not work up, purified directly. The residue was purified by prep-HPLC (neutral condition) to give title compound (Compound 132) (9 mg, 24.07 μmol, 4.09% yield, 96.5% purity) as a white solid. LCMS (ES⁺): m/z 361.3 [M+H]⁺.

### Example 230. Synthesis of 5-(4-(2-((1-acetylpiperidin-4-yl)amino)-5-chloropyrimidin-4-yl)-1H-pyrazol-1-yl)pyridin-2(1H)-one (Compound 110)

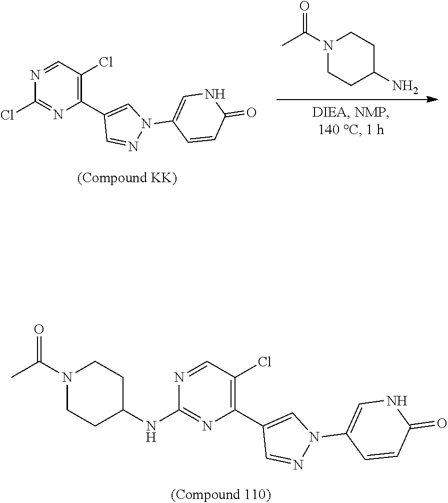

Synthesis of 5-(4-(2-((1-acetylpiperidin-4-yl)amino)-5-chloropyrimidin-4-yl)-1H-pyrazol-1-yl)pyridin-2(1H)-one (Compound 110). To a solution of 1-(4-aminopiperidin-1-yl)ethan-1-one (123.82 mg, 642.60 μmol, 1.1 eq, HCl) in NMP (4 mL) was added 5-[4-(2, 5-dichloropyrimidin-4-yl) pyrazol-1-yl]-1H-pyridin-2-one (Compound KK) (180 mg, 584.18 μmol, 1 eq) and DIEA (377.51 mg, 2.92 mmol, 508.77 μL, 5 eq). The mixture was stirred at 140 °C for 1 h. LCMS showed 5-[4-(2, 5-dichloropyrimidin-4-yl) pyrazol-1-yl]-1H-pyridin-2-one (Compound KK) was consumed completely and 19.7% peak with desired mass was detected. The reaction did not work up, purified directly. The resulting solution was purified by prep-HPLC (FA condition, column: Phenomenex luna C18 150*25 mm*10 um; mobile phase: [water(FA)-ACN]; gradient: 14%-44% B over 10 min) to give title compound (Compound 110) (41 mg, 96.58 μmol, 14.88% yield, 97.49% purity) as a white solid. LCMS (ES⁺): m/z 414.0 [M+H]⁺.

### Example 231. Synthesis of 4-(4-(2-((1-acetylpiperidin-4-yl)amino)-5-chloropyrimidin-4-yl)-1H-pyrazol-1-yl)pyridin-2(1H)-one (Compound 120)

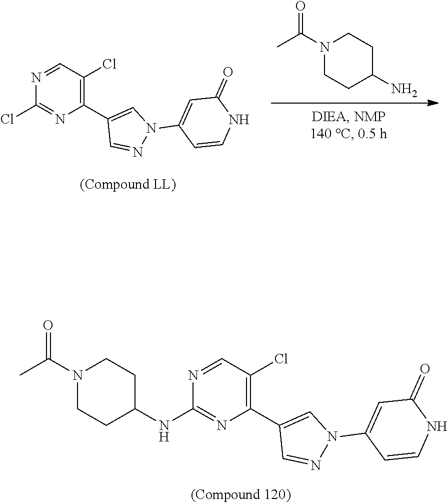

Synthesis of 4-(4-(2-((1-acetylpiperidin-4-yl)amino)-5-chloropyrimidin-4-yl)-1H-pyrazol-1-yl)pyridin-2(1H)-one (Compound 120). To a solution of 1-(4-aminopiperidin-1-yl)ethan-1-one (103.18 mg, 535.50 μmol, 1.1 eq, HCl) in NMP (4 mL) was added DIEA (188.75 mg, 1.46 mmol, 254.39 μL, 3 eq) and 4-[4-(2,5-dichloropyrimidin-4-yl)pyrazol-1-yl]-1H-pyridin-2-one (Compound LL) (150 mg, 486.82 μmol, 1 eq). The mixture was stirred at 140 °C for 0.5 h. LCMS showed 4-[4-(2,5-dichloropyrimidin-4-yl)pyrazol-1-yl]-1H-pyridin-2-one (Compound LL) was consumed completely and desired mass was detected. The product was dissolved in DMF (2 mL) and filtered to removed the insoluble. The sample was purified by prep-HPLC (FA condition; or neutral condition; or basic condition, column: Welch Ultimate C18 150*25 mm*5 um; mobile phase: [water (FA)-ACN]; gradient: 18%-48% B over 10 min) to give title compound (Compound 120) (40.9 mg, 96.25 μmol, 19.77% yield, 97.39% purity) as a white solid. LCMS (ES⁺): m/z 414.0 [M+H]⁺.

### Example 232. Synthesis of 3-(4-(2-((1-acetylpiperidin-4-yl)amino)-5-chloropyrimidin-4-yl)-1H-pyrazol-1-yl)pyridin-2(1H)-one (Compound 111)

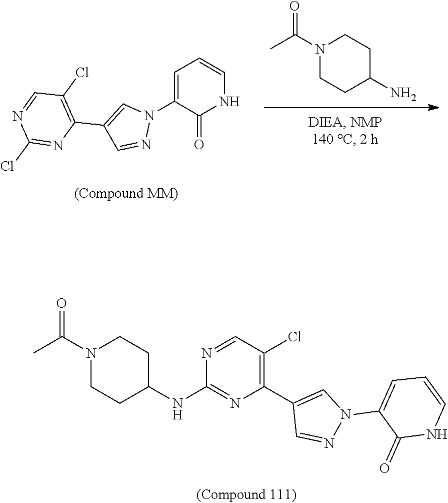

Synthesis of 3-(4-(2-((1-acetylpiperidin-4-yl)amino)-5-chloropyrimidin-4-yl)-1H-pyrazol-1-yl)pyridin-2(1H)-one (Compound 111). To a solution of 3-[4-(2,5-dichloropyrimidin-4-yl)pyrazol-1-yl]-1H-pyridin-2-one (Compound MM)) (200 mg, 649.09 mol, 1 eq) in NMP (3 mL) was added DIEA (419.45 mg, 3.25 mmol, 565.30 μL, 5 eq) and 1-(4-aminopiperidin-1-yl)ethan-1-one (111.54 mg, 578.89 μmol, 8.92e-1 eq, HCl). The mixture was stirred at 140 °C for 2 h. LCMS showed 3-[4-(2,5-dichloropyrimidin-4-yl)pyrazol-1-yl]-1H-pyridin-2-one (Compound MM)) was consumed completely and the desired mass was detected. After filtration, vacuum distillation removes solvents to obtain crude products. The crude products was purified by prep-HPLC (column: Welch Xtimate C18 150*25 mm*5 um; mobile phase: [water (FA)-ACN]; gradient: 17%-47% B over 10 min) to give compound (Compound 111) (12.7 mg, 29.23 μmol, 9.01% yield, 95.26% purity) as a yellow solid. LCMS (ES+): m/z 414.0 [M+H]⁺.

### Example 233. Synthesis of cis-N-((1s,4s)-4-((5-chloro-4-(6-(4-fluorophenyl)pyridin-2-yl)pyrimidin-2-yl)amino)cyclohexyl)acetamide (Compound 130)

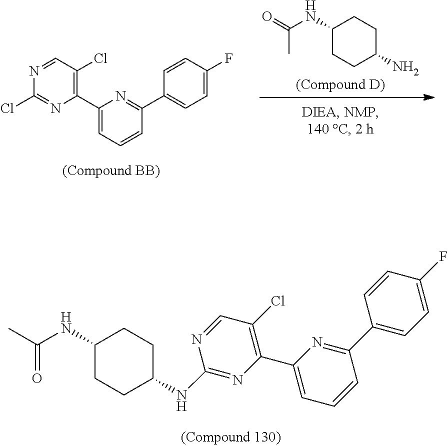 [SMILES: Fc1ccc(-c2cccc(-c3nc(Cl)ncc3Cl)n2)cc1 ; CC(=O)N[C@H]1CC[C@@H](N)CC1 ; CC(=O)N[C@H]1CC[C@@H](Nc2ncc(Cl)c(-c3cccc(-c4ccc(F)cc4)n3)n2)CC1]

Synthesis of cis-N-((1s,4s)-4-((5-chloro-4-(6-(4-fluorophenyl)pyridin-2-yl)pyrimidin-2-yl)amino)cyclohexyl)acetamide (Compound 130). To a solution of 2,5-dichloro-4-(6-(4-fluorophenyl)pyridin-2-yl)pyrimidine (Compound BB) (0.1 g, 312.36 μmol, 1 eq) in NMP (2 mL) was added DIEA (201.85 mg, 1.56 mmol, 272.03 μL, 5 eq) and cis-N-((1s,4s)-4-aminocyclohexyl)acetamide (Compound D) (60.19 mg, 312.36 μmol, 1 eq, HCl). The mixture was stirred at 140 °C for 2 h. LCMS showed 2,5-dichloro-4-(6-(4-fluorophenyl)pyridin-2-yl)pyrimidine (Compound BB) was consumed completely and desired mass was detected. The reaction did not work up, purified directly. The reaction mixture was dissolved in DMF (3 mL), and the resulting solution was purified by prep-HPLC (FA condition, column: Welch Xtimate C18 150*25 mm*5 um; mobile phase: [water(FA)-ACN]; gradient: 50%-80% B over 10 min) to give title compound (Compound 130) (24.5 mg, 54.94 μmol, 17.59% yield, 98.65% purity) as a white solid. LCMS (ES+): m/z 440.1 [M+H]⁺.

### Example 234. Synthesis of cis-N-((1s,4s)-4-((5-chloro-4-(1-cyclopropyl-1H-pyrazol-4-yl)pyrimidin-2-yl)amino)cyclohexyl)acetamide (Compound 99)

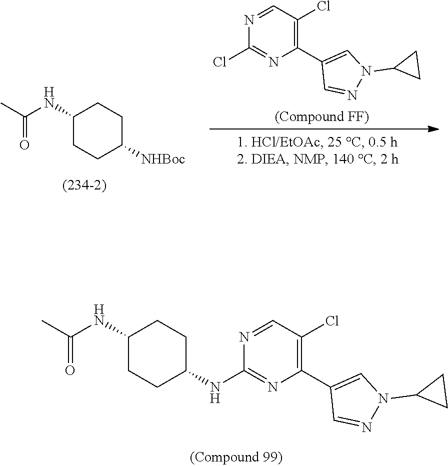 [SMILES: Clc1ncc(Cl)c(-c2cnn(C3CC3)c2)n1 ; CC(=O)N[C@H]1CC[C@@H](NC(=O)OC(C)(C)C)CC1 ; CC(=O)N[C@H]1CC[C@@H](Nc2ncc(Cl)c(-c3cnn(C4CC4)c3)n2)CC1]

Synthesis of cis-N-((1s, 4s)-4-((5-chloro-4-(1-cyclopropyl-1H-pyrazole-4-yl) pyrimidin-2-yl) amino) cyclohexyl) acetamide (Compound 99). To a solution of cis-tert-butyl ((1s,4s)-4-acetamidocyclohexyl)carbamate (234-2) (150.73 mg, 588.00 μmol, 1 eq) in HCl/EtOAc (2 mL), the mixture was stirred at 25 °C for 0.5 h. The reaction mixture was concentrated under reduced pressure to give a residue and then the residue in NMP (3 mL) was added 2, 5-dichloro-4-(1-cyclopropylpyrazol-4-yl)pyrimidine (Compound FF) (0.15 g, 588.00 μmol, 1 eq) and DIEA (227.98 mg, 1.76 mmol, 307.26 μL, 3 eq) at 25 °C, the mixture was stirred at 140 °C for 2 h. LCMS showed 2, 5-dichloro-4-(1-cyclopropylpyrazol-4-yl)pyrimidine (Compound FF) was consumed completely and the desired mass was detected. The residue was purified by prep-HPLC (neutral condition; column: column: Phenomenex C18 150*25 mm*10 um; mobile phase: [water (NH₄HCO₃)-ACN]; gradient: 26%-56% B over 14 min) to give title compound (Compound 99) (0.030 g, 79.08 μmol, 13.45% yield, 98.82% purity) as a white solid. LCMS (ES+): m/z 375.1 [M+H]⁺.

### Example 235. Synthesis of trans-N-((1r,4r)-4-((5-chloro-4-(6-(4-fluorophenyl)pyridin-2-yl)pyrimidin-2-yl)amino)cyclohexyl)acetamide (Compound 158)

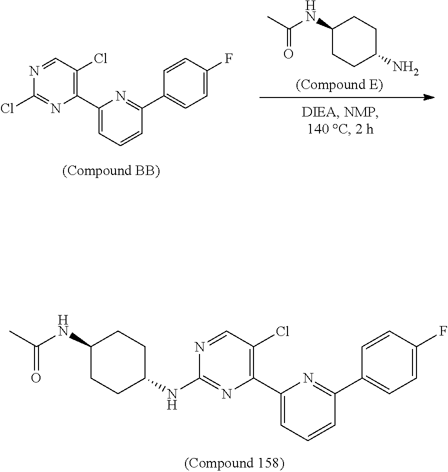 [SMILES: Fc1ccc(-c2cccc(-c3nc(Cl)ncc3Cl)n2)cc1 ; CC(=O)N[C@H]1CC[C@H](N)CC1 ; CC(=O)N[C@H]1CC[C@H](Nc2ncc(Cl)c(-c3cccc(-c4ccc(F)cc4)n3)n2)CC1]

Synthesis of trans-N-((1r,4r)-4-((5-chloro-4-(6-(4-fluorophenyl)pyridin-2-yl)pyrimidin-2-yl)amino)cyclohexyl)acetamide (Compound 158). To a solution of 2,5-dichloro-4-(6-(4-fluorophenyl)pyridin-2-yl)pyrimidine (Compound BB) (0.1 g, 312.36 μmol, 1 eq) in NMP (2 mL) was added DIEA (201.85 mg, 1.56 mmol, 272.03 μL, 5 eq) and trans-N-((1r,4r)-4-aminocyclohexyl)acetamide (Compound E) (60.19 mg, 312.36 μmol, 1 eq, HCl). The mixture was stirred at 140 °C for 2 h. LCMS showed 2,5-dichloro-4-(6-(4-fluorophenyl)pyridin-2-yl)pyrimidine (Compound BB) was consumed completely and desired mass was detected. The reaction did not work up, purified directly. The reaction mixture was dissolved in DMF (5 mL), and the resulting solution was purified by prep-HPLC (FA condition, column: Phenomenex luna C18 150*25 mm*10 um; mobile phase: [water (FA)-ACN]; gradient: 45%-75% B over 10 min) to give title compound (Compound 158) (20.2 mg, 45.46 μmol, 14.56% yield, 99.01% purity) as a white solid. LCMS (ES⁺): m/z 440.1 [M+H]⁺.

### Example 236. Synthesis of trans-N-((1r,4r)-4-((5-chloro-4-(2-(4-fluorophenyl)pyridin-4-yl)pyrimidin-2-yl)amino)cyclohexyl)acetamide (Compound 142)

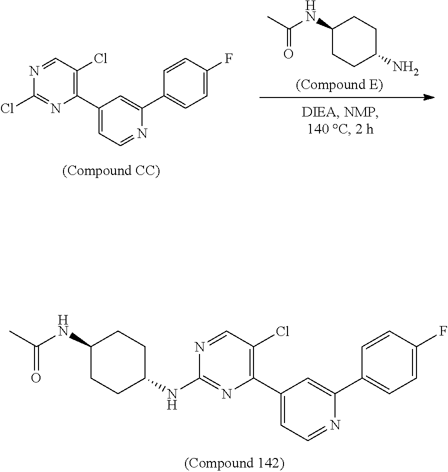 [SMILES: Fc1ccc(-c2cc(-c3nc(Cl)ncc3Cl)ccn2)cc1 ; CC(=O)N[C@H]1CC[C@H](N)CC1 ; CC(=O)N[C@H]1CC[C@H](Nc2ncc(Cl)c(-c3ccnc(-c4ccc(F)cc4)c3)n2)CC1]

Synthesis of trans-N-((1r,4r)-4-((5-chloro-4-(2-(4-fluorophenyl)pyridin-4-yl)pyrimidin-2-yl)amino)cyclohexyl)acetamide (Compound 142). To a solution of 2,5-dichloro-4-(2-(4-fluorophenyl)pyridin-4-yl)pyrimidine (Compound CC) (0.1 g, 312.36 μmol, 1 eq) in NMP (2 mL) was added DIEA (201.85 mg, 1.56 mmol, 272.03 μL, 5 eq) and trans-N-((1r,4r)-4-aminocyclohexyl)acetamide (Compound E) (60.19 mg, 312.36 μmol, 1 eq, HCl). The mixture was stirred at 140 °C for 2 h. LCMS showed 2,5-dichloro-4-(2-(4-fluorophenyl)pyridin-4-yl)pyrimidine (Compound CC) was consumed completely and desired mass was detected. The reaction did not work up, purified directly. The reaction mixture was dissolved in DMF (5 mL), and the resulting solution was purified by prep-HPLC (FA condition, column: Phenomenex luna C18 150*25 mm*10 um; mobile phase: [water(FA)-ACN]; gradient: 43%-73% B over 10 min) to give title compound (Compound 142) (52.2 mg, 117.25 μmol, 37.54% yield, 98.81% purity) as a white solid. LCMS (ES⁺): m/z 440.0 [M+H]⁺.

### Example 237. Synthesis of trans-N-((1r,4r)-4-((5-chloro-4-(1-cyclopropyl-6-oxo-1,6-dihydropyridin-3-yl)pyrimidin-2-yl)amino)cyclohexyl)acetamide (Compound 96)

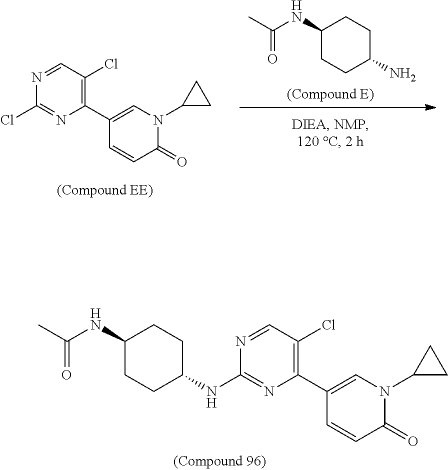 [SMILES: O=c1ccc(-c2nc(Cl)ncc2Cl)cn1C1CC1 ; CC(=O)N[C@H]1CC[C@H](N)CC1 ; CC(=O)N[C@H]1CC[C@H](Nc2ncc(Cl)c(-c3ccc(=O)n(C4CC4)c3)n2)CC1]

Synthesis of trans-N-((1r,4r)-4-((5-chloro-4-(1-cyclopropyl-6-oxo-1,6-dihydropyridin-3-yl)pyrimidin-2-yl)amino)cyclohexyl)acetamide (Compound 96). To a solution of 1-cyclopropyl-5-(2,5-dichloropyrimidin-4-yl)pyridin-2(1H)-one (Compound EE) (180 mg, 638.02 μmol, 1 eq) in NMP (3 mL) was added DIEA (412.29 mg, 3.19 mmol, 555.65 μL, 5 eq) and trans-N-((1r,4r)-4-aminocyclohexyl)acetamide (Compound E) (119.61 mg, 765.62 μmol, 1.2 eq). The mixture was stirred at 120 °C for 2 h. LCMS showed 1-cyclopropyl-5-(2,5-dichloropyrimidin-4-yl)pyridin-2(1H)-one (Compound EE) was consumed completely and the desired mass was detected. After filtration, vacuum distillation removes solvents to obtain crude products. The residue was purified by prep-HPLC (column: Welch Ultimate C18 150*25 mm*5 um; mobile phase: [water (FA)-ACN]; gradient: 18%-48% B over 10 min) to give title compound (Compound 96) (80.7 mg, 197.23 μmol, 30.91% yield, 98.22% purity) as a pale brown solid. LCMS (ES⁺): m/z 402.0 [M+H]⁺.

### Example 238. Synthesis of trans-N-((1r,4r)-4-((5-chloro-4-(1-cyclopropyl-1H-pyrazol-4-yl)pyrimidin-2-yl)amino)cyclohexyl)acetamide (Compound 116)

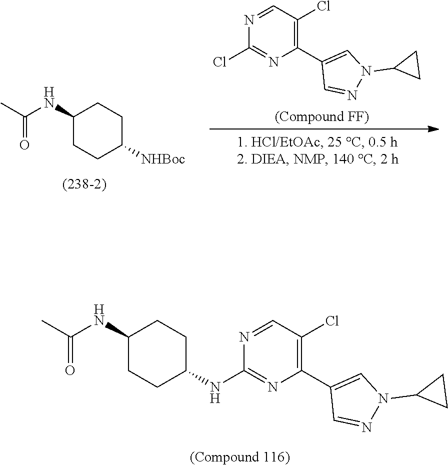 [SMILES: Clc1ncc(Cl)c(-c2cnn(C3CC3)c2)n1 ; CC(=O)N[C@H]1CC[C@H](NC(=O)OC(C)(C)C)CC1 ; CC(=O)N[C@H]1CC[C@H](Nc2ncc(Cl)c(-c3cnn(C4CC4)c3)n2)CC1]

Synthesis of trans-N-((1r,4r)-4-((5-chloro-4-(I-cyclopropyl-1H-pyrazol-4-yl)pyrimidin-2-yl)amino)cyclohexyl)acetamide (Compound 116). To a solution of trans-tert-butyl ((1r,4r)-4-acetamidocyclohexyl)carbamate (238-2) (134.23 mg, 588.00 μmol, 1 eq) in HCl/EtOAc (2 mL), the mixture was stirred at 25 °C for 0.5 h. The reaction mixture was concentrated under reduced pressure to give a residue and then the residue in NMP (2 mL) was added 2,5-dichloro-4-(1-cyclopropyl-1H-pyrazol-4-yl)pyrimidine (Compound FF) (0.15 g, 588.00 μmol, 1 eq) and DIEA (380 mg, 2.94 mmol, 512 μL, 5 eq) at 25 °C, the mixture was stirred at 140 °C for 2 h. LCMS showed 2,5-dichloro-4-(1-cyclopropyl-1H-pyrazol-4-yl)pyrimidine (Compound FF) was consumed completely and the desired mass was detected. The reaction did not work up, purified directly. The residue was purified by prep-HPLC (neutral condition; column: column: Phenomenex C18 150*25 mm*10 um; mobile phase: [water (NH₄HCO₃)-ACN]; gradient: 26%-56% B over 14 min) to give title compound (Compound 116) (0.032 g, 90.98 μmol, 15.47% yield, 98.6% purity) as a white solid. LCMS (ES⁺): m/z 375.2 [M+H]⁺.

### Example 239. Synthesis of trans-N-((1r,4r)-4-((5-chloro-4-(5-cyclopropyl-1H-pyrazol-3-yl)pyrimidin-2-yl)amino)cyclohexyl)acetamide (Compound 190)

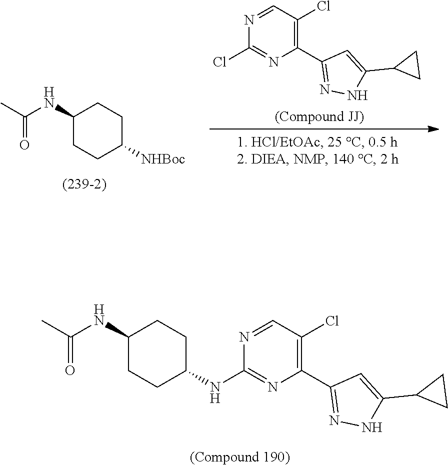 [SMILES: Clc1ncc(Cl)c(-c2cc(C3CC3)[nH]n2)n1 ; CC(=O)N[C@H]1CC[C@H](NC(=O)OC(C)(C)C)CC1 ; CC(=O)N[C@H]1CC[C@H](Nc2ncc(Cl)c(-c3cc(C4CC4)[nH]n3)n2)CC1]

Synthesis of trans-N-((1r,4r)-4-((5-chloro-4-(5-cyclopropyl-1H-pyrazol-3-yl)pyrimidin-2-yl)amino)cyclohexyl)acetamide (Compound 190). A solution of trans-tert-butyl ((1r,4r)-4-acetamidocyclohexyl)carbamate (239-2) (200.97 mg, 784.00 μmol, 1 eq) in HCl/EtOAc (3 mL) was stirred at 25 °C for 0.5 h. The reaction mixture was concentrated under reduced pressure to give a residue and then the residue in NMP (3 mL) was added 2,5-dichloro-4-(5-cyclopropyl-1H-pyrazol-3-yl)pyrimidine (Compound JJ) (0.2 g, 784.00 μmol, 1 eq) and DIEA (101.33 mg, 784.00 μmol, 136.56 μL, 1 eq) at 25 °C and the mixture was stirred at 140 °C for 2 h. LCMS showed 2,5-dichloro-4-(5-cyclopropyl-1H-pyrazol-3-yl)pyrimidine (Compound JJ) was consumed completely and the desired mass was detected. The residue was purified by prep-HPLC (neutral condition) to give title compound (Compound 190) (0.005 g, 13.15 μmol, 1.68% yield, 98.6% purity) as a white solid. LCMS (ES⁺): m/z 375.3 [M+H]⁺.

### Example 240. Synthesis of trans-N-((1r,4r)-4-((5-chloro-4-(1-(6-oxo-1,6-dihydropyridin-3-yl)-1H-pyrazol-4-yl)pyrimidin-2-yl)amino)cyclohexyl)acetamide (Compound 145)

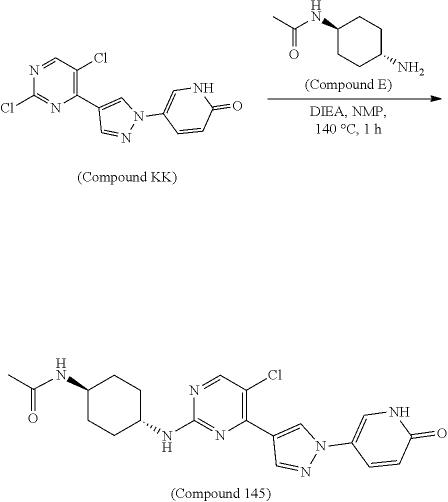 [SMILES: O=c1ccc(-n2cc(-c3nc(Cl)ncc3Cl)cn2)c[nH]1 ; CC(=O)N[C@H]1CC[C@H](N)CC1 ; CC(=O)N[C@H]1CC[C@H](Nc2ncc(Cl)c(-c3cnn(-c4ccc(=O)[nH]c4)c3)n2)CC1]

Synthesis of trans-N-((1r,4r)-4-((5-chloro-4-(1-(6-oxo-1,6-dihydropyridin-3-yl)-1H-pyrazol-4-yl)pyrimidin-2-yl)amino)cyclohexyl)acetamide (Compound 145). To a solution of trans-N-((1r,4r)-4-aminocyclohexyl)acetamide (Compound E) (100.06 mg, 519.27 μmol, 1 eq, HCl) in NMP (3 mL) was added 5-(4-(2,5-dichloropyrimidin-4-yl)-1H-pyrazol-1-yl)pyridin-2(5H)-one (Compound KK) (160 mg, 519.27 μmol, 1 eq) and DIEA (335.56 mg, 2.60 mmol, 452.24 μL, 5 eq). The mixture was stirred at 140 °C for 1 h. LCMS showed 5-(4-(2,5-dichloropyrimidin-4-yl)-1H-pyrazol-1-yl)pyridin-2(5H)-one (Compound KK) was consumed completely and 76.9% product with desired mass was detected. The product was dissolved in DMF (2 mL) and filtered to remove the insoluble. The sample was purified by prep-HPLC (FA condition; column: Phenomenex luna C18 150*25 mm*10 um; mobile phase: [water (FA)-ACN]; gradient: 14%-44% B over 10 min) to give title compound (Compound 145) (36.7 mg, 84.73 μmol, 16.32% yield, 98.79% purity) as a white solid. LCMS (ES⁺): m/z 428.1 [M+H]⁺.

### Example 241. Synthesis of trans-N-((1r,4r)-4-((5-chloro-4-(1-(2-oxo-1,2-dihydropyridin-4-yl)-1H-pyrazol-4-yl)pyrimidin-2-yl)amino)cyclohexyl)acetamide (Compound 147)

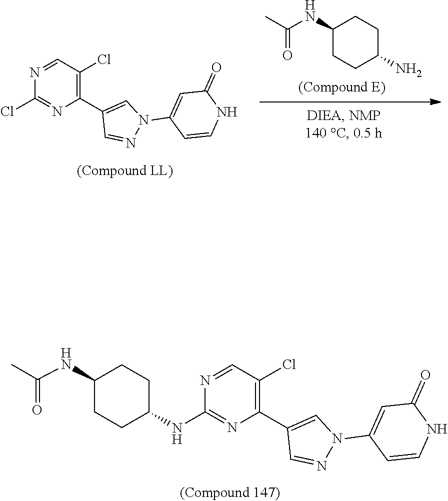 [SMILES: O=c1cc(-n2cc(-c3nc(Cl)ncc3Cl)cn2)cc[nH]1 ; CC(=O)N[C@H]1CC[C@H](N)CC1 ; CC(=O)N[C@H]1CC[C@H](Nc2ncc(Cl)c(-c3cnn(-c4cc[nH]c(=O)c4)c3)n2)CC1]

Synthesis of trans-N-((1r,4r)-4-((5-chloro-4-(-(2-oxo-1,2-dihydropyridin-4-yl)-1H-pyrazol-4-yl)pyrimidin-2-yl)amino)cyclohexyl)acetamide (Compound 147). To a solution of trans-N-((1r,4r)-4-aminocyclohexyl)acetamide (Compound E) (103.18 mg, 535.50 μmol, 1.1 eq, HCl) in NMP (3 mL) was added DIEA (314.59 mg, 2.43 mmol, 423.98 μL, 5 eq) and 4-(4-(2,5-dichloropyrimidin-4-yl)-1H-pyrazol-1-yl)pyridin-2(1H)-one (Compound LL) (150 mg, 486.82 μmol, 1 eq). The mixture was stirred at 140 °C for 0.5 h. LCMS showed 4-(4-(2,5-dichloropyrimidin-4-yl)-1H-pyrazol-1-yl)pyridin-2(1H)-one (Compound LL) was consumed completely and 67% product with desired mass was detected. The product was dissolved in DMF (2 mL) and filtered to remove the insoluble. The sample was purified by prep-HPLC (FA condition; or neutral condition; or basic condition, column: Phenomenex luna C18 150*25 mm*10 um; mobile phase: [water (FA)-ACN]; gradient: 15%-45% B over 5 min) to give title compound (Compound 147) (15.4 mg, 35.48 μmol, 7.29% yield, 98.58% purity) as a white solid. LCMS (ES⁺): m/z 428.1 [M+H]⁺.

### Example 242. Synthesis of trans-N-((1r,4r)-4-((5-chloro-4-(1-(2-oxo-1,2-dihydropyridin-3-yl)-1H-pyrazol-4-yl)pyrimidin-2-yl)amino)cyclohexyl)acetamide (Compound 149)

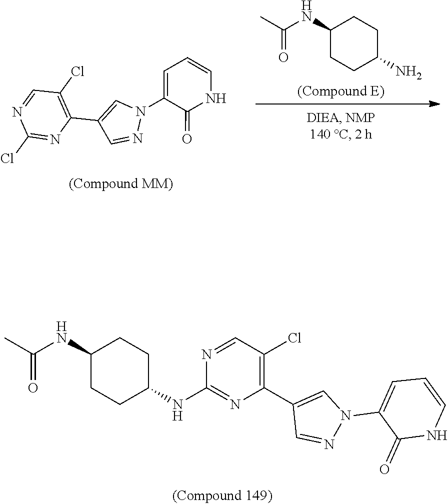 [SMILES: O=c1[nH]cccc1-n1cc(-c2nc(Cl)ncc2Cl)cn1 ; CC(=O)N[C@H]1CC[C@H](N)CC1 ; CC(=O)N[C@H]1CC[C@H](Nc2ncc(Cl)c(-c3cnn(-c4ccc[nH]c4=O)c3)n2)CC1]

Synthesis of trans-N-((1r,4r)-4-((5-chloro-4-(1-(2-oxo-1,2-dihydropyridin-3-yl)-1H-pyrazol-4-yl)pyrimidin-2-yl)amino)cyclohexyl)acetamide (Compound 149). To a solution of 3-(4-(2,5-dichloropyrimidin-4-yl)-1H-pyrazol-1-yl)pyridin-2(1H)-one (Compound MM)) (200 mg, 649.09 μmol, 1 eq) in NMP (3 mL) was added DIEA (419.45 mg, 3.25 mmol, 565.30 μL, 5 eq) and trans-N-((1r,4r)-4-aminocyclohexyl)acetamide (Compound E) (111.54 mg, 578.89 μmol, 8.92e-1 eq, HCl). The mixture was stirred at 140 °C for 2 h. LCMS showed 3-(4-(2,5-dichloropyrimidin-4-yl)-1H-pyrazol-1-yl)pyridin-2(1H)-one (Compound MM)) was consumed completely and the desired mass was detected. After filtration, vacuum distillation removes solvents to obtain crude products. The crude products were purified by prep-HPLC (column: Welch Xtimate C18 150*25 mm*5 um; mobile phase: [water (FA)-ACN]; gradient: 17%-47% B over 10 min) to give title compound (Compound 149) (12.8 mg, 28.80 μmol, 4.44% yield, 96.29% purity) as a yellow solid. LCMS (ES⁺): m/z 428.2 [M+H]⁺.

### Example 243. Synthesis of trans-N-((1r,4r)-4-((5-chloro-4-(1-(4-fluorophenyl)-1H-pyrazol-4-yl)pyrimidin-2-yl)amino)cyclohexyl)acetamide (Compound 121)

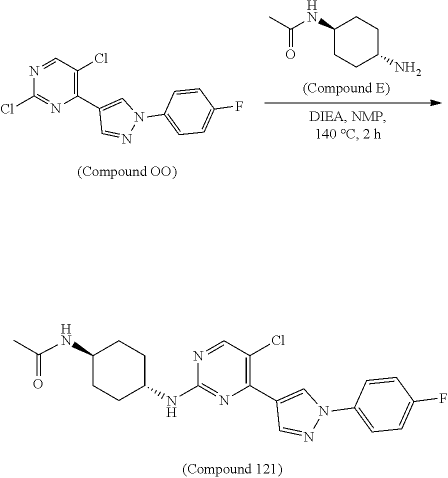 [SMILES: Fc1ccc(-n2cc(-c3nc(Cl)ncc3Cl)cn2)cc1 ; CC(=O)N[C@H]1CC[C@H](N)CC1 ; CC(=O)N[C@H]1CC[C@H](Nc2ncc(Cl)c(-c3cnn(-c4ccc(F)cc4)c3)n2)CC1]

Synthesis of trans-N-((1r,4r)-4-((5-chloro-4-(1-(4-fluorophenyl)-1H-pyrazol-4-yl)pyrimidin-2-yl)amino)cyclohexyl)acetamide (Compound 121). To a solution of trans-N-((1r,4r)-4-aminocyclohexyl)acetamide (Compound E) (98.55 mg, 630.81 μmol, 1.3 eq) in NMP (2 mL) was added DIEA (313.57 mg, 2.43 mmol, 422.60 μL, 5 eq) and 2,5-dichloro-4-(1-(4-fluorophenyl)-1H-pyrazol-4-yl)pyrimidine (Compound 00) (150 mg, 485.24 μmol, 1 eq). The mixture was stirred at 140 °C for 2 h. LCMS showed 2,5-dichloro-4-(1-(4-fluorophenyl)-1H-pyrazol-4-yl)pyrimidine (Compound 00) was consumed completely and 57.8% product with desired mass was detected. After filtration, vacuum distillation removes solvents to obtain crude products. The crude products were purified by prep-HPLC (FA condition; column: Welch Ultimate C18 150*25 mm*5 um; mobile phase: [water (FA)-ACN]; gradient: 43%-73% B over 10 min) to give title compound (Compound 121) (62.0 mg, 139.96 μmol, 28.84% yield, 96.82% purity) as a white solid. LCMS (ES⁺): m/z 429.0 [M+H]⁺.

### Example 244. Synthesis of trans-(1r,4r)-4-((5-chloro-4-(4′-fluoro-[1,1′-biphenyl]-3-yl)pyrimidin-2-yl)amino)-N-methylcyclohexane-1-carboxamide (Compound 101)

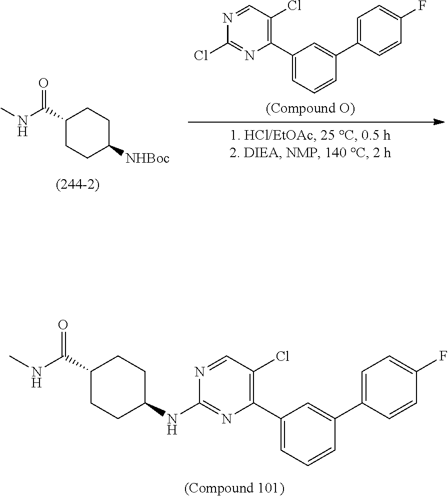 [SMILES: CNC(=O)[C@H]1CC[C@H](NC(=O)OC(C)(C)C)CC1 ; Fc1ccc(-c2cccc(-c3nc(Cl)ncc3Cl)c2)cc1 ; CNC(=O)[C@H]1CC[C@H](Nc2ncc(Cl)c(-c3cccc(-c4ccc(F)cc4)c3)n2)CC1]

Synthesis of trans-(1r,4r)-4-((5-chloro-4-(4′ fluoro-[1,1′-biphenyl]-3-yl)pyrimidin-2-yl)amino)-N-methylcyclohexane-1-carboxamide (Compound 101). To a solution of trans-tert-butyl ((1r,4r)-4-(methylcarbamoyl)cyclohexyl)carbamate (244-2) (160.63 mg, 626.64 μmol, 1 eq) in HCl/EtOAc (5 mL) was stirred at 25 °C for 0.5 h. The reaction mixture was concentrated under reduced pressure to give a residue and then the residue in NMP (2 mL) was added 2,5-dichloro-4-(4′-fluoro-[1,1′-biphenyl]-3-yl)pyrimidine (Compound O) (200 mg, 626.64 μmol, 1 eq) and DIEA (404.95 mg, 3.13 mmol, 545.75 μL, 5 eq) at 25 °C, the mixture was stirred at 140 °C for 2 h. LCMS showed 2,5-dichloro-4-(4′-fluoro-[1,1′-biphenyl]-3-yl)pyrimidine (Compound O) was consumed completely and desired mass was detected. The residue was purified by prep-HPLC: (FA condition; column: Welch Ultimate C18 150*25 mm*5 um; mobile phase: [water(FA)-ACN]; gradient: 56%-86% B over 10 min) to give title compound (Compound 101) (33.7 mg, 76.78 μmol, 12.25% yield) as a white solid. LCMS (ES+): m/z 439.0 [M+H]⁺.

### Example 245. Synthesis of trans-(1r,4r)-4-((5-chloro-4-(3-(6-oxo-1,6-dihydropyridin-2-yl)phenyl)pyrimidin-2-yl)amino)-N-methylcyclohexane-1-carboxamide (Compound 92)

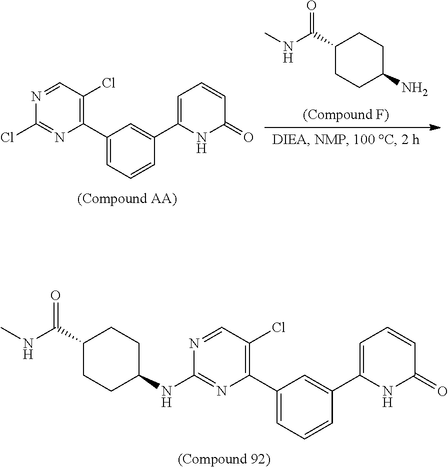 [SMILES: O=c1cccc(-c2cccc(-c3nc(Cl)ncc3Cl)c2)[nH]1 ; CNC(=O)[C@H]1CC[C@H](N)CC1 ; CNC(=O)[C@H]1CC[C@H](Nc2ncc(Cl)c(-c3cccc(-c4cccc(=O)[nH]4)c3)n2)CC1]

Synthesis of trans-(1r,4r)-4-((5-chloro-4-(3-(6-oxo-1,6-dihydropyridin-2-yl)phenyl)pyrimidin-2-yl)amino)-N-methylcyclohexane-1-carboxamide (Compound 92). To a solution of 6-(3-(2,5-dichloropyrimidin-4-yl)phenyl)pyridin-2(1H)-one (Compound AA) (200 mg, 628.62 μmol, 1 eq) in NMP (2 mL) was added trans-(1r,4r)-4-amino-N-methylcyclohexane-1-carboxamide (Compound F) (98.21 mg, 628.62 μmol, 1 eq), DIEA (406.22 mg, 3.14 mmol, 547.47 μL, 5 eq) under N₂, and the reaction mixture was stirred at 100 °C for 2 h under N₂. LCMS showed 6-(3-(2,5-dichloropyrimidin-4-yl)phenyl)pyridin-2 (1H)-one (Compound AA) was consumed completely and desired mass was detected. The reaction mixture was filtered and the filtrate was concentrated to give a residue. The residue was purified by reversed-phase HPLC (column: Phenomenex luna C18 150*25 mm*10 um; mobile phase: [water(FA)-ACN]; gradient: 22%-52% B over 10 min) to give title compound (Compound 92) (51.3 mg, 115.20 μmol, 18.33% yield, 98.342% purity) as a pale gray solid. LCMS (ES⁺): m/z 438.0 [M+H]⁺.

### Example 246. Synthesis of trans-(1r,4r)-4-((5-chloro-4-(6-(4-fluorophenyl)pyridin-2-yl)pyrimidin-2-yl)amino)-N-methylcyclohexane-1-carboxamide (Compound 159)

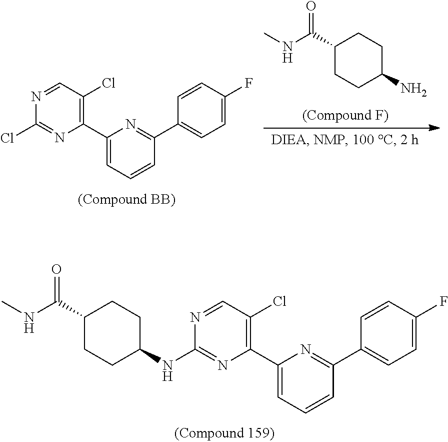 [SMILES: Fc1ccc(-c2cccc(-c3nc(Cl)ncc3Cl)n2)cc1 ; CNC(=O)[C@H]1CC[C@H](N)CC1 ; CNC(=O)[C@H]1CC[C@H](Nc2ncc(Cl)c(-c3cccc(-c4ccc(F)cc4)n3)n2)CC1]

Synthesis of trans-(1r,4r)-4-((5-chloro-4-(6-(4-fluorophenyl)pyridin-2-yl)pyrimidin-2-yl)amino)-N-methylcyclohexane-1-carboxamide (Compound 159). To a solution of 2,5-dichloro-4-(6-(4-fluorophenyl)pyridin-2-yl)pyrimidine (Compound BB) (0.1 g, 312.36 μmol, 1 eq) in NMP (2 mL) was added DIEA (201.85 mg, 1.56 mmol, 272.03 μL, 5 eq) and trans-(1r,4r)-4-amino-N-methylcyclohexane-1-carboxamide (Compound F) (60.19 mg, 312.36 μmol, 1 eq, HCl). The mixture was stirred at 100 °C for 2 h. LC-MS showed 2,5-dichloro-4-(6-(4-fluorophenyl)pyridin-2-yl)pyrimidine (Compound BB) was consumed completely and desired mass was detected. The reaction did not work up, purified directly. The reaction mixture was dissolved in DMF (5 mL), and the resulting solution was purified by prep-HPLC (FA condition, column: Welch Xtimate C18 150*25 mm*5 um; mobile phase: [water(FA)-ACN]; gradient: 41%-71% B over 9 min) to give title compound (Compound 159) (26.5 mg, 59.04 μmol, 18.90% yield, 98.01% purity) as a white solid. LCMS (ES⁺): m/z 440.1 [M+H]⁺.

### Example 247. Synthesis of trans-(1r,4r)-4-((5-chloro-4-(2-(4-fluorophenyl)pyridin-4-yl)pyrimidin-2-yl)amino)-N-methylcyclohexane-1-carboxamide (Compound 160)

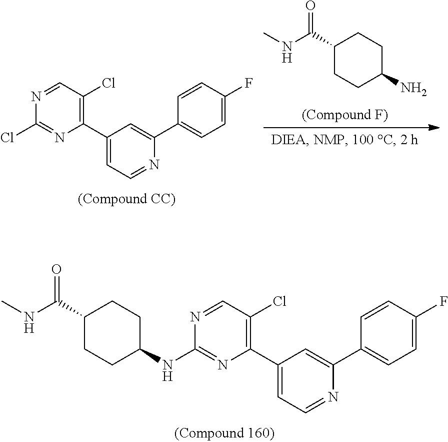 [SMILES: Fc1ccc(-c2cc(-c3nc(Cl)ncc3Cl)ccn2)cc1 ; CNC(=O)[C@H]1CC[C@H](N)CC1 ; CNC(=O)[C@H]1CC[C@H](Nc2ncc(Cl)c(-c3ccnc(-c4ccc(F)cc4)c3)n2)CC1]

Synthesis of trans-(1r,4r)-4-((5-chloro-4-(2-(4-fluorophenyl)pyridin-4-yl)pyrimidin-2-yl)amino)-N-methylcyclohexane-1-carboxamide (Compound 160). To a solution of 2,5-dichloro-4-(2-(4-fluorophenyl)pyridin-4-yl)pyrimidine (Compound CC) (0.1 g, 312.36 μmol, 1 eq) in NMP (2 mL) was added DIEA (201.85 mg, 1.56 mmol, 272.03 μL, 5 eq) and trans-(1r,4r)-4-amino-N-methylcyclohexane-1-carboxamide (Compound F) (60.19 mg, 312.36 μmol, 1 eq, HCl). The mixture was stirred at 140 °C for 2 h. LCMS showed 2,5-dichloro-4-(2-(4-fluorophenyl)pyridin-4-yl)pyrimidine (Compound CC) was consumed completely and desired mass was detected. The reaction did not work up, purified directly. The reaction mixture was dissolved in DMF (5 mL), and the resulting solution was purified by prep-HPLC (FA condition, column: Phenomenex luna C18 150*25 mm*10 um; mobile phase: [water(FA)-ACN]; gradient: 41%-71% B over 10 min) to give title compound (Compound 160) (43.3 mg, 93.87 μmol, 30.05% yield, 95.37% purity) as a white solid. LCMS (ES⁺): m/z 440.1 [M+H]⁺.

### Example 248. Synthesis of trans-(1r,4r)-4-((5-chloro-4-(1-(4-fluorophenyl)-6-oxo-1,6-dihydropyridin-3-yl)pyrimidin-2-yl)amino)-N-methylcyclohexane-1-carboxamide (Compound 95)

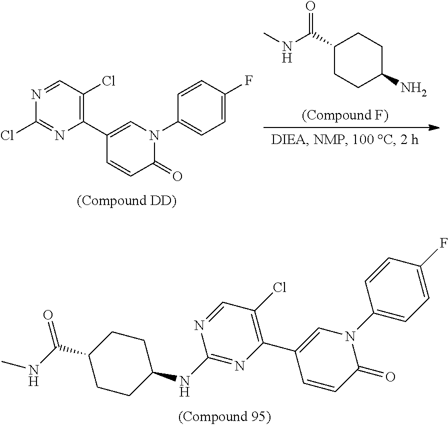 [SMILES: O=c1ccc(-c2nc(Cl)ncc2Cl)cn1-c1ccc(F)cc1 ; CNC(=O)[C@H]1CC[C@H](N)CC1 ; CNC(=O)[C@H]1CC[C@H](Nc2ncc(Cl)c(-c3ccc(=O)n(-c4ccc(F)cc4)c3)n2)CC1]

Synthesis of trans-(1r,4r)-4-((5-chloro-4-(1-(4-fluorophenyl)-6-oxo-1,6-dihydropyridin-3-yl)pyrimidin-2-yl)amino)-N-methylcyclohexane-1-carboxamide (Compound 95). To a solution of 5-(2,5-dichloropyrimidin-4-yl)-1-(4-fluorophenyl)pyridin-2(1H)-one (Compound DD) (0.1 g, 297.49 μmol, 1 eq) in NMP (3 mL) was added DIEA (192.24 mg, 1.49 mmol, 259.09 μL, 5 eq) and trans-(1r,4r)-4-amino-N-methylcyclohexane-1-carboxamide (Compound F). The mixture was stirred at 100 °C for 2 h. LCMS showed 5-(2,5-dichloropyrimidin-4-yl)-1-(4-fluorophenyl)pyridin-2(1H)-one (Compound DD) was consumed completely and desired mass was detected. The reaction did not work up, purified directly. The reaction mixture was dissolved in DMF (2 mL), and the resulting solution was purified by prep-HPLC (FA condition, column: Welch Xtimate C18 150*25 mm*5 um; mobile phase: [water (FA)-ACN]; gradient: 25%-55% B over 0 min) to give title compound (Compound 95) (30 mg, 65.80 μmol, 22.12% yield, 100% purity) as a white solid. LCMS (ES⁺): m/z 456.2 [M+H]⁺.

### Example 249. Synthesis of trans-(1r,4r)-4-((5-chloro-4-(1-cyclopropyl-6-oxo-1,6-dihydropyridin-3-yl)pyrimidin-2-yl)amino)-N-methylcyclohexane-1-carboxamide (Compound 106)

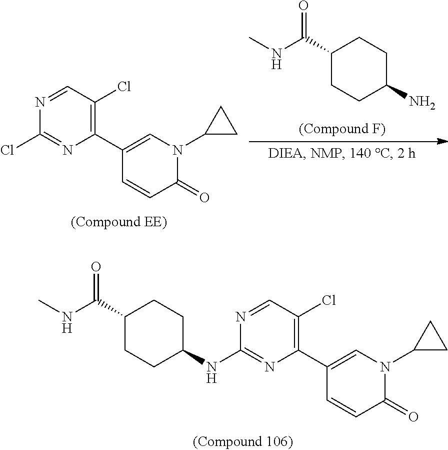 [SMILES: O=c1ccc(-c2nc(Cl)ncc2Cl)cn1C1CC1 ; CNC(=O)[C@H]1CC[C@H](N)CC1 ; CNC(=O)[C@H]1CC[C@H](Nc2ncc(Cl)c(-c3ccc(=O)n(C4CC4)c3)n2)CC1]

Synthesis of trans-(1r,4r)-4-((5-chloro-4-(I-cyclopropyl-6-oxo-1,6-dihydropyridin-3-yl)pyrimidin-2-yl)amino)-N-methylcyclohexane-1-carboxamide (Compound 106). To a solution of 1-cyclopropyl-5-(2,5-dichloropyrimidin-4-yl)pyridin-2(1H)-one (Compound EE) (200 mg, 708.91 μmol, 1 eq) in NMP (3 mL) was added DIEA (458.11 mg, 3.54 mmol, 617.39 μL, 5 eq) and trans-(1r,4r)-4-amino-N-methylcyclohexane-1-carboxamide (Compound F) (110.75 mg, 708.91 μmol, 1 eq). The mixture was stirred at 140 °C for 2 h. LCMS showed 1-cyclopropyl-5-(2,5-dichloropyrimidin-4-yl)pyridin-2(1H)-one (Compound EE) was consumed completely and 70.5% product with desired mass was detected. After filtration, vacuum distillation removes solvents to obtain crude products. The residue was purified by prep-HPLC (basic condition; column: Welch Ultimate C18 150*25 mm*5 um; mobile phase: [water(FA)-ACN]; gradient: 18%-48% B over 10 min) to give title compound (Compound 106) (26.1 mg, 62.90 μmol, 8.87% yield, 96.85% purity) as a white solid. LCMS (ES⁺): m/z 402.0 [M+H]⁺.

### Example 250. Synthesis of trans-(1r,4r)-4-((5-chloro-4-(1-cyclopropyl-1H-pyrazol-4-yl)pyrimidin-2-yl)amino)-N-methylcyclohexane-1-carboxamide (Compound 117)

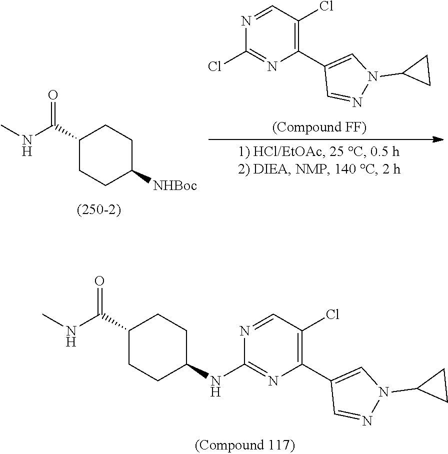 [SMILES: CNC(=O)[C@H]1CC[C@H](NC(=O)OC(C)(C)C)CC1 ; Clc1ncc(Cl)c(-c2cnn(C3CC3)c2)n1 ; CNC(=O)[C@H]1CC[C@H](Nc2ncc(Cl)c(-c3cnn(C4CC4)c3)n2)CC1]

Synthesis of trans-(1r,4r)-4-((5-chloro-4-(I-cyclopropyl-1H-pyrazol-4-yl)pyrimidin-2-yl)amino)-N-methylcyclohexane-1-carboxamide (Compound 117). To a solution of trans-tert-butyl ((1r,4r)-4-(methylcarbamoyl)cyclohexyl)carbamate (250-2) (150.73 mg, 588.00 μmol, 1 eq) in HCl/EtOAc (2 mL), the mixture was stirred at 25 °C for 0.5 h. The reaction mixture was concentrated under reduced pressure to give a residue and then the residue in NMP (3 mL) was added 2,5-dichloro-4-(1-cyclopropyl-1H-pyrazol-4-yl)pyrimidine (Compound FF) (0.15 g, 588.00 μmol, 1 eq) and DIEA (75.99 mg, 588.00 μmol, 102.42 μL, 1 eq) at 25 °C, the mixture was stirred at 140 °C for 2 h. LCMS showed 2,5-dichloro-4-(1-cyclopropyl-1H-pyrazol-4-yl)pyrimidine (Compound FF) was consumed completely and the desired mass was detected. The residue was purified by prep-HPLC (neutral condition; column: Waters xbridge 150*25 mm 10 um; mobile phase: [water (NH₄HCO₃)-ACN]; gradient: 37%-67% B over 10 min) to give title compound (Compound 117) (0.014 g, 36.79 μmol, 6.26% yield, 98.5% purity) as a white solid. LCMS (ES+): m/z 375.3 [M+H]⁺.

### Example 251. Synthesis of trans-(1r,4r)-4-((5-chloro-4-(1-cyclohexyl-1H-pyrazol-4-yl)pyrimidin-2-yl)amino)-N-methylcyclohexane-1-carboxamide (Compound 118)

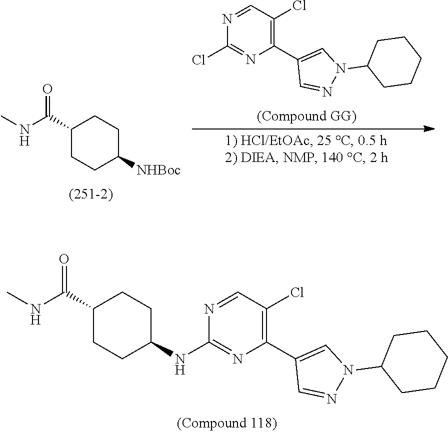 [SMILES: CNC(=O)[C@H]1CC[C@H](NC(=O)OC(C)(C)C)CC1 ; Clc1ncc(Cl)c(-c2cnn(C3CCCCC3)c2)n1 ; CNC(=O)[C@H]1CC[C@H](Nc2ncc(Cl)c(-c3cnn(C4CCCCC4)c3)n2)CC1]

Synthesis of trans-(1r,4r)-4-((5-chloro-4-(1-cyclohexyl-1H-pyrazol-4-yl)pyrimidin-2-yl)amino)-N-methylcyclohexane-1-carboxamide (Compound 118). To a solution of trans-tert-butyl ((1r,4r)-4-(methylcarbamoyl)cyclohexyl)carbamate (251-2) (150.73 mg, 588.00 μmol, 1 eq) in HCl/EtOAc (2 mL), the mixture was stirred at 25 °C for 0.5 h. The reaction mixture was concentrated under reduced pressure to give a residue and then the residue in NMP (3 mL) was added 2,5-dichloro-4-(1-cyclohexyl-1H-pyrazol-4-yl)pyrimidine (Compound GG) (0.15 g, 588.00 μmol, 1 eq) and DIEA (75.99 mg, 588.00 μmol, 102.42 μL, 1 eq) at 25 °C, the mixture was stirred at 140 °C for 2 h. LCMS showed 2,5-dichloro-4-(1-cyclohexyl-1H-pyrazol-4-yl)pyrimidine (Compound GG) was consumed completely and the desired mass was detected. The residue was purified by prep-HPLC (neutral condition; column: Waters xbridge 150*25 mm 10 um; mobile phase: [water (NH₄HCO₃)-ACN]; gradient: 37%-67% B over 10 min) to give title compound (Compound 118) (0.014 g, 36.79 μmol, 6.26% yield, 98.5% purity) as a white solid. LCMS (ES+): m/z 417.3 [M+H]⁺.

### Example 252. Synthesis of trans-(1r,4r)-4-((5-chloro-4-(5-cyclopropyl-1H-pyrazol-3-yl)pyrimidin-2-yl)amino)-N-methylcyclohexane-1-carboxamide (Compound 144)

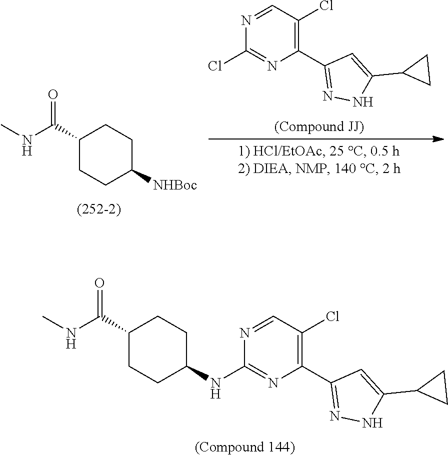 [SMILES: CNC(=O)[C@H]1CC[C@H](NC(=O)OC(C)(C)C)CC1 ; Clc1ncc(Cl)c(-c2cc(C3CC3)[nH]n2)n1 ; CNC(=O)[C@H]1CC[C@H](Nc2ncc(Cl)c(-c3cc(C4CC4)[nH]n3)n2)CC1]

Synthesis of trans-(1r,4r)-4-((5-chloro-4-(5-cyclopropyl-1H-pyrazol-3-yl)pyrimidin-2-yl)amino)-N-methylcyclohexane-1-carboxamide (Compound 144). To a solution of trans-tert-butyl ((1r,4r)-4-(methylcarbamoyl)cyclohexyl)carbamate (252-2) in HCl/EtOAc (3 mL), the mixture was stirred at 25 °C for 0.5 h. The reaction mixture was concentrated under reduced pressure to give a residue and then the residue in NMP (3 mL) was added 2,5-dichloro-4-(5-cyclopropyl-1H-pyrazol-3-yl)pyrimidine (Compound JJ) (0.15 g, 588.00 μmol, 1 eq) and DIEA (75.99 mg, 588.00 μmol, 102.42 μL, 1 eq) at 25 °C, the mixture was stirred at 140 °C for 2 h. LCMS showed 2,5-dichloro-4-(5-cyclopropyl-1H-pyrazol-3-yl)pyrimidine (Compound JJ) was consumed completely and the desired mass was detected. The residue was purified by prep-HPLC (neutral condition) to give title compound (Compound 144) (70 mg, 177.02 μmol, 30.11% yield, 94.8% purity) as a white solid. LCMS (ES+): m/z 375.3 [M+H]⁺.

### Example 253. Synthesis of trans-(1r,4r)-4-((5-chloro-4-(1-(6-oxo-1,6-dihydropyridin-3-yl)-1H-pyrazol-4-yl)pyrimidin-2-yl)amino)-N-methylcyclohexane-1-carboxamide (Compound 146)

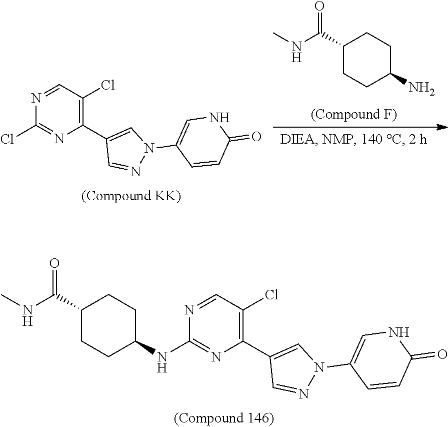 [SMILES: O=c1ccc(-n2cc(-c3nc(Cl)ncc3Cl)cn2)c[nH]1 ; CNC(=O)[C@H]1CC[C@H](N)CC1 ; CNC(=O)[C@H]1CC[C@H](Nc2ncc(Cl)c(-c3cnn(-c4ccc(=O)[nH]c4)c3)n2)CC1]

Synthesis of trans-(1r,4r)-4-((5-chloro-4-(1-(6-oxo-1,6-dihydropyridin-3-yl)-1H-pyrazol-4-yl)pyrimidin-2-yl)amino)-N-methylcyclohexane-1-carboxamide (Compound 146). To a solution of trans-(1r,4r)-4-amino-N-methylcyclohexane-1-carboxamide (Compound F) (100 mg, 518.98 μmol, 8.88e-1 eq, HCl) in NMP (3 mL) was added 5-(4-(2,5-dichloropyrimidin-4-yl)-1H-pyrazol-1-yl)pyridin-2(1H)-one (Compound KK) (180 mg, 584.18 μmol, 1 eq) and DIEA (226.50 mg, 1.75 mmol, 305.26 μL, 3 eq). The mixture was stirred at 140 °C for 2 h. LCMS showed 5-(4-(2,5-dichloropyrimidin-4-yl)-1H-pyrazol-1-yl)pyridin-2(1H)-one (Compound KK) was consumed completely and 40% product with desired mass was detected. The product was dissolved in DMF (2 mL) and filtered to removed the insoluble. The sample was purified by prep-HPLC (HCl condition; column: Welch Xtimate C18 150*25 mm*5 um; mobile phase: [water(HCl)-ACN]; gradient: 15%-35% B over 10 min) to give title compound (Compound 146) (7.8 mg, 17.66 μmol, 3.02% yield, 96.90% purity) as a yellow solid. LCMS (ES+): m/z 428.3 [M+H]⁺.

### Example 254. Synthesis of trans-(1r,4r)-4-((5-chloro-4-(1-(2-oxo-1,2-dihydropyridin-4-yl)-1H-pyrazol-4-yl)pyrimidin-2-yl)amino)-N-methylcyclohexane-1-carboxamide (Compound 165)

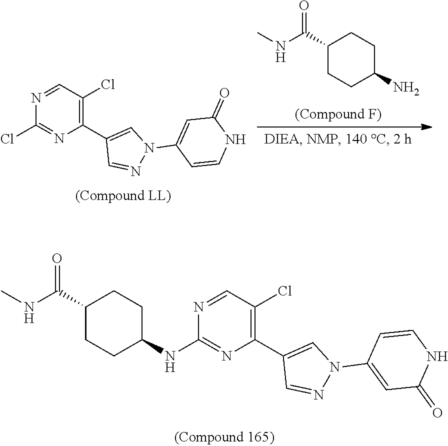 [SMILES: O=c1cc(-n2cc(-c3nc(Cl)ncc3Cl)cn2)cc[nH]1 ; CNC(=O)[C@H]1CC[C@H](N)CC1 ; CNC(=O)[C@H]1CC[C@H](Nc2ncc(Cl)c(-c3cnn(-c4cc[nH]c(=O)c4)c3)n2)CC1]

Synthesis of trans-(1r,4r)-4-((5-chloro-4-(1-(2-oxo-1,2-dihydropyridin-4-yl)-1H-pyrazol-4-yl)pyrimidin-2-yl)amino)-N-methylcyclohexane-1-carboxamide (Compound 165). To a solution of trans-(1r,4r)-4-amino-N-methylcyclohexane-1-carboxamide (Compound F) (116.17 mg, 602.90 μmol, 1.1 eq, HCl) in NMP (3 mL) was added DIEA (212.51 mg, 1.64 mmol, 286.40 μL, 3 eq) and 4-(4-(2,5-dichloropyrimidin-4-yl)-1H-pyrazol-1-yl)pyridin-2 (1H)-one (Compound LL) (168.88 mg, 548.09 μmol, 1 eq). The mixture was stirred at 140 °C for 0.5 h. LCMS showed 4-(4-(2,5-dichloropyrimidin-4-yl)-1H-pyrazol-1-yl)pyridin-2 (1H)-one (Compound LL) (168.88 mg, 548.09 μmol, 1 eq) was consumed completely and 49.85% product with desired mass was detected. The product was dissolved in DMF (2 mL) and filtered to removed the insoluble. The residue was purified by prep-HPLC (HCl condition; or neutral condition; or basic condition, column: Welch Ultimate C18 150*25 mm*5 um; mobile phase: [water(HCl)-ACN]; gradient: 15%-35% B over 10 min) to give title compound (Compound 165) (10.1 mg, 22.15 μmol, 4.04% yield, 93.85% purity) as a white solid. LCMS (ES+): m/z 428.1 [M+H]⁺.

### Example 255. Synthesis of trans-(1r,4r)-4-((5-chloro-4-(1-(4-fluorophenyl)-1H-pyrazol-4-yl)pyrimidin-2-yl)amino)-N-methylcyclohexane-1-carboxamide (Compound 134)

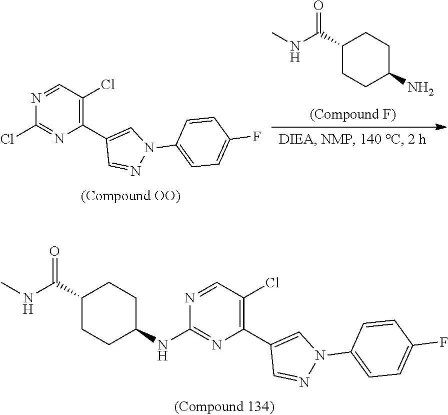 [SMILES: Fc1ccc(-n2cc(-c3nc(Cl)ncc3Cl)cn2)cc1 ; CNC(=O)[C@H]1CC[C@H](N)CC1 ; CNC(=O)[C@H]1CC[C@H](Nc2ncc(Cl)c(-c3cnn(-c4ccc(F)cc4)c3)n2)CC1]

Synthesis of trans-(1r,4r)-4-((5-chloro-4-(1-(4-fluorophenyl)-1H-pyrazol-4-yl)pyrimidin-2-yl)amino)-N-methylcyclohexane-1-carboxamide (Compound 134). To a solution of trans-(1r,4r)-4-amino-N-methylcyclohexane-1-carboxamide (Compound F) (98.55 mg, 511.45 μmol, 1.05 eq, HCl) in NMP (4 mL) was added 2,5-dichloro-4-(1-(4-fluorophenyl)-1H-pyrazol-4-yl)pyrimidine (Compound 00) (150 mg, 485.24 μmol, 1 eq) and DIEA (62.71 mg, 485.24 μmol, 84.52 μL, 1 eq). The mixture was stirred at 140 °C for 2 h. LCMS showed 2,5-dichloro-4-(1-(4-fluorophenyl)-1H-pyrazol-4-yl)pyrimidine (Compound 00) was consumed completely and 56.0% peak with desired mass was detected. The reaction did not work up, purified directly. The resulting solution was purified by prep-HPLC (FA condition, column: Phenomenex luna C18 150*25 mm*10 um; mobile phase: [water(FA)-ACN]; gradient: 42%-72% B over 10 min) to give title compound (Compound 134) (22.7 mg, 51.53 μmol, 10.62% yield, 97.36% purity) as a white solid. LCMS (ES+): m/z 429.2 [M+H]⁺.

### Example 256. Synthesis of cis-(1s,4s)-4-((5-chloro-4-(6-(4-fluorophenyl)pyridin-2-yl)pyrimidin-2-yl)amino)-N-methylcyclohexane-1-carboxamide (Compound 131)

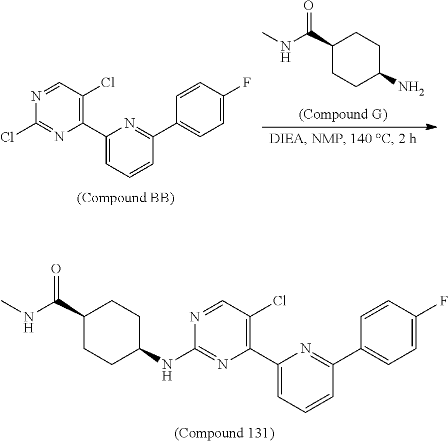 [SMILES: Fc1ccc(-c2cccc(-c3nc(Cl)ncc3Cl)n2)cc1 ; CNC(=O)[C@H]1CC[C@@H](N)CC1 ; CNC(=O)[C@H]1CC[C@@H](Nc2ncc(Cl)c(-c3cccc(-c4ccc(F)cc4)n3)n2)CC1]

Synthesis of cis-(1s,4s)-4-((5-chloro-4-(6-(4-fluorophenyl)pyridin-2-yl)pyrimidin-2-yl)amino)-N-methylcyclohexane-1-carboxamide (Compound 131). To a solution of 2,5-dichloro-4-(6-(4-fluorophenyl)pyridin-2-yl)pyrimidine (Compound BB) (0.1 g, 312.36 μmol, 1 eq) in NMP (2 mL) was added DIEA (201.85 mg, 1.56 mmol, 272.03 μL, 5 eq) and cis-(1s,4s)-4-amino-N-methylcyclohexane-1-carboxamide (Compound G) (60.19 mg, 312.36 μmol, 1 eq, HCl). The mixture was stirred at 140 °C for 2 h. LCMS showed 2,5-dichloro-4-(6-(4-fluorophenyl)pyridin-2-yl)pyrimidine (Compound BB) was consumed completely and desired mass was detected. The reaction did not work up, purified directly. The reaction mixture was dissolved in DMF (3 mL), and the resulting solution was purified by prep-HPLC (FA condition, column: Welch Xtimate C18 150*25 mm*5 um; mobile phase: [water (FA)-ACN]; gradient: 52%-82% B over 10 min) to give title compound (Compound 131) (28.3 mg, 62.59 μmol, 20.04% yield, 97.29% purity) as a white solid. LCMS (ES+): m/z 440.0 [M+H]⁺.

### Example 257. Synthesis of cis-(1s,4s)-4-((5-chloro-4-(1-cyclopropyl-1H-pyrazol-4-yl)pyrimidin-2-yl)amino)-N-methylcyclohexane-1-carboxamide (Compound 107)

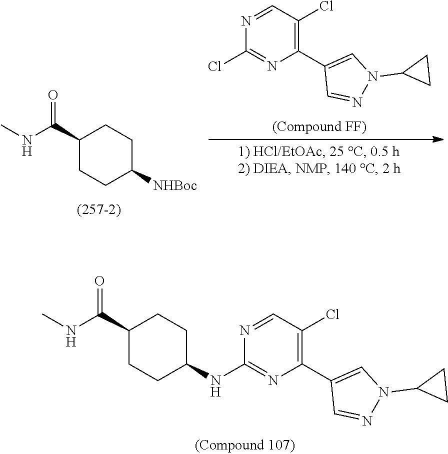 [SMILES: CNC(=O)[C@H]1CC[C@@H](NC(=O)OC(C)(C)C)CC1 ; Clc1ncc(Cl)c(-c2cnn(C3CC3)c2)n1 ; CNC(=O)[C@H]1CC[C@@H](Nc2ncc(Cl)c(-c3cnn(C4CC4)c3)n2)CC1]

Synthesis of cis-(1s,4s)-4-((5-chloro-4-(1-cyclopropyl-1H-pyrazol-4-yl)pyrimidin-2-yl)amino)-N-methylcyclohexane-1-carboxamide (Compound 107). A solution of cis-tert-butyl N-[4-(methylcarbamoyl) cyclohexyl]carbamate (257-2) (150.73 mg, 588.00 μmol, 1 eq) in HCl/EtOAc (2 mL) was stirred at 25 °C for 0.5 h. The reaction mixture was concentrated under reduced pressure to give a residue and then the residue in NMP (3 mL) was added 2,5-dichloro-4-(1-cyclopropyl-1H-pyrazol-4-yl)pyrimidine (Compound FF) (150 mg, 588.00 μmol, 1 eq) and DIEA (227.98 mg, 1.76 mmol, 307.26 μL, 3 eq) at 25 °C and the mixture was stirred at 140 °C for 2 h. LCMS showed 2,5-dichloro-4-(1-cyclopropyl-1H-pyrazol-4-yl)pyrimidine (Compound FF) was consumed completely and the desired mass was detected. The reaction did not work up, purified directly. The residue was purified by prep-HPLC (neutral condition; column:column: Phenomenex C18 150*25 mm*10 um; mobile phase: [water(NH₄HCO₃)-ACN]; gradient: 26%-56% B over 14 min) to give title compound (Compound 107) (0.042 g, 109.56 μmol, 18.63% yield, 97.79% purity) as a white solid. LCMS (ES+): m/z 375.3 [M+H]⁺.

### Example 258. Synthesis of cis-(1s,4s)-4-((5-chloro-4-(1-cyclohexyl-1H-pyrazol-4-yl)pyrimidin-2-yl)amino)-N-methylcyclohexane-1-carboxamide (Compound 108)

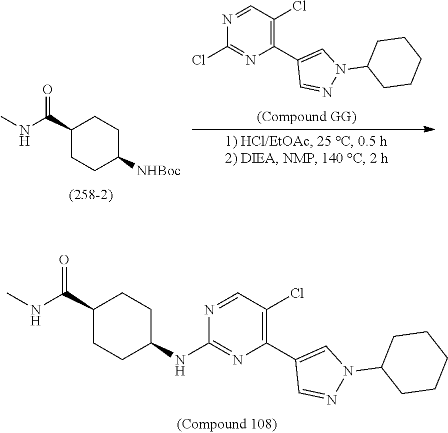 [SMILES: CNC(=O)[C@H]1CC[C@@H](NC(=O)OC(C)(C)C)CC1 ; Clc1ncc(Cl)c(-c2cnn(C3CCCCC3)c2)n1 ; CNC(=O)[C@H]1CC[C@@H](Nc2ncc(Cl)c(-c3cnn(C4CCCCC4)c3)n2)CC1]

Synthesis of cis-(1s,4s)-4-((5-chloro-4-(1-cyclohexyl-1H-pyrazol-4-yl)pyrimidin-2-yl)amino)-N-methylcyclohexane-1-carboxamide (Compound 108). To a solution of cis-tert-butyl N-[4-(methylcarbamoyl) cyclohexyl]carbamate (258-2) (0.15 g, 504.74 μmol, 1 eq) in HCl/EtOAc (2 mL), the mixture was stirred at 25 °C for 0.5 h. The reaction mixture was concentrated under reduced pressure to give a residue and then the residue in NMP (3 mL) was added tert-butyl ((1s,4s)-4-(methylcarbamoyl)cyclohexyl)carbamate (Compound GG) (129.39 mg, 504.74 μmol, 1 eq) and DIEA (195.70 mg, 1.51 mmol, 263.75 μL, 3 eq) at 25 °C. The mixture was stirred at 140 °C for 2 h. LCMS showed tert-butyl ((1s,4s)-4-(methylcarbamoyl)cyclohexyl)carbamate (Compound GG) was consumed completely and the desired mass was detected. The reaction did not work up, purified directly. The residue was purified by prep-HPLC (neutral condition; column:column: Phenomenex C18 150*25 mm*10 um; mobile phase: [water (NH₄HCO₃)-ACN]; gradient: 26%-56% B over 14 min) to give title compound (Compound 108) (0.028 g, 65.19 μmol, 12.92% yield, 97.07% purity) as a white solid. LCMS (ES+): m/z 417.3 [M+H]⁺.

### Example 259. Synthesis of cis-(1s,4s)-4-((5-chloro-4-(5-cyclopropyl-1H-pyrazol-3-yl)pyrimidin-2-yl)amino)-N-methylcyclohexane-1-carboxamide (Compound 191)

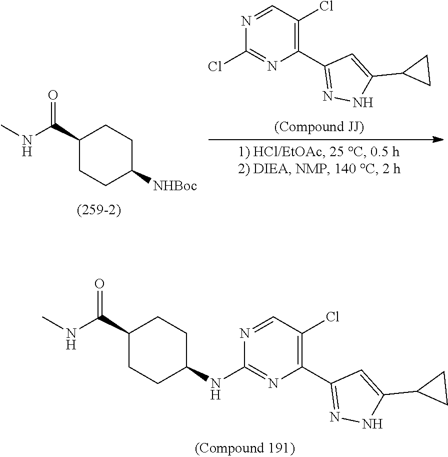 [SMILES: CNC(=O)[C@H]1CC[C@@H](NC(=O)OC(C)(C)C)CC1 ; Clc1ncc(Cl)c(-c2cc(C3CC3)[nH]n2)n1 ; CNC(=O)[C@H]1CC[C@@H](Nc2ncc(Cl)c(-c3cc(C4CC4)[nH]n3)n2)CC1]

Synthesis of cis-(1s,4s)-4-((5-chloro-4-(5-cyclopropyl-1H-pyrazol-3-yl)pyrimidin-2-yl)amino)-N-methylcyclohexane-1-carboxamide (Compound 191). To a solution of cis-tert-butyl N-[4-(methylcarbamoyl) cyclohexyl]carbamate (259-2) (200.97 mg, 784.00 μmol, 1 eq) in HCl/EtOAc (3 mL) was stirred at 25 °C for 0.5 h. The reaction mixture was concentrated under reduced pressure to give a residue and then the residue in NMP (3 mL) was added 2,5-dichloro-4-(5-cyclopropyl-1H-pyrazol-3-yl)pyrimidine (Compound JJ) (0.2 g, 784.00 μmol, 1 eq) and DIEA (101.33 mg, 784.00 μmol, 136.56 μL, 1 eq) at 25 °C. The mixture was stirred at 140 °C for 2 h. LCMS showed 2,5-dichloro-4-(5-cyclopropyl-1H-pyrazol-3-yl)pyrimidine (Compound JJ) was consumed completely and the desired mass was detected. The reaction did not work up, purified directly. The residue was purified by prep-HPLC (neutral condition) to give title compound (Compound 191) (0.007 g, 18.30 μmol, 2.33% yield, 98% purity) as a white solid. LCMS (ES+): m/z 375.2 [M+H]⁺.

### Example 260. Synthesis of cis-(1s,4s)-4-((5-chloro-4-(1-(6-oxo-1,6-dihydropyridin-3-yl)-1H-pyrazol-4-yl)pyrimidin-2-yl)amino)-N-methylcyclohexane-1-carboxamide (Compound 163)

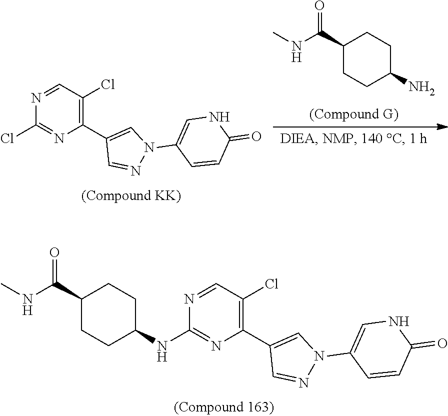 [SMILES: O=c1ccc(-n2cc(-c3nc(Cl)ncc3Cl)cn2)c[nH]1 ; CNC(=O)[C@H]1CC[C@@H](N)CC1 ; CNC(=O)[C@H]1CC[C@@H](Nc2ncc(Cl)c(-c3cnn(-c4ccc(=O)[nH]c4)c3)n2)CC1]

Synthesis of cis-(1s,4s)-4-((5-chloro-4-(1-(6-oxo-1,6-dihydropyridin-3-yl)-1H-pyrazol-4-yl)pyrimidin-2-yl)amino)-N-methylcyclohexane-1-carboxamide (Compound 163). To a solution of cis-(1s,4s)-4-amino-N-methylcyclohexane-1-carboxamide (Compound G) (123.82 mg, 642.60 μmol, 1.1 eq, HCl) in NMP (4 mL) was added 5-(4-(2,5-dichloropyrimidin-4-yl)-1H-pyrazol-1-yl)pyridin-2(1H)-one (Compound KK) (180 mg, 584.18 μmol, 1 eq) and DIEA (377.51 mg, 2.92 mmol, 508.77 μL, 5 eq). The mixture was stirred at 140 °C for 1 h. LCMS showed 5-(4-(2,5-dichloropyrimidin-4-yl)-1H-pyrazol-1-yl)pyridin-2(1H)-one (Compound KK) was consumed completely and 19.7% peak with desired mass was detected. The reaction did not work up, purified directly. The resulting solution was purified by prep-HPLC (FA condition, column: Phenomenex luna C18 150*25 mm*10 um; mobile phase: [water(FA)-ACN]; gradient: 14%-44% B over 10 min) to give title compound (Compound 163) (10.4 mg, 23.97 μmol, 4.10% yield, 98.63% purity) as a white solid. LCMS (ES+): m/z 428.3 [M+H]⁺.

### Example 261. Synthesis of cis-(1s,4s)-4-((5-chloro-4-(1-(2-oxo-1,2-dihydropyridin-4-yl)-1H-pyrazol-4-yl)pyrimidin-2-yl)amino)-N-methylcyclohexane-1-carboxamide (Compound 148)

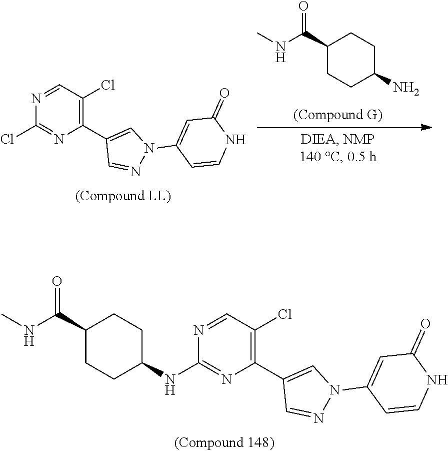 [SMILES: O=c1cc(-n2cc(-c3nc(Cl)ncc3Cl)cn2)cc[nH]1 ; CNC(=O)[C@H]1CC[C@@H](N)CC1 ; CNC(=O)[C@H]1CC[C@@H](Nc2ncc(Cl)c(-c3cnn(-c4cc[nH]c(=O)c4)c3)n2)CC1]

Synthesis of cis-(1s,4s)-4-((5-chloro-4-(1-(2-oxo-1,2-dihydropyridin-4-yl)-1H-pyrazol-4-yl)pyrimidin-2-yl)amino)-N-methylcyclohexane-1-carboxamide (Compound 148). To a solution of cis-(1s, 4s)-4-amino-N-methylcyclohexane-1-carboxamide (Compound G) (103.18 mg, 535.50 μmol, 1.1 eq, HCl) in NMP (4 mL) was added DIEA (188.75 mg, 1.46 mmol, 254.39 μL, 3 eq) and 4-(4-(2,5-dichloropyrimidin-4-yl)-1H-pyrazol-1-yl)pyridin-2 (1H)-one (Compound LL) (150 mg, 486.82 μmol, 1 eq). The mixture was stirred at 140 °C for 0.5 h. LCMS showed 4-(4-(2,5-dichloropyrimidin-4-yl)-1H-pyrazol-1-yl)pyridin-2(1H)-one (Compound LL) was consumed completely and desired mass was detected. The product was dissolved in DMF (2 mL) and filtered to removed the insoluble. The sample was purified by prep-HPLC (FA condition; or neutral condition; or basic condition, column: Welch Ultimate C18 150*25 mm*5 um; mobile phase: [water(FA)-ACN]; gradient: 18%-48% B over 10 min) to give title compound (Compound 148) (56.36 mg, 125.03 μmol, 25.68% yield, 94.92% purity) as a white solid. LCMS (ES⁺): m/z 428.3 [M+H]⁺.

### Example 262. Synthesis of cis-(1s,4s)-4-((5-chloro-4-(1-(6-oxo-1,6-dihydropyridin-2-yl)-1H-pyrazol-4-yl)pyrimidin-2-yl)amino)-N-methylcyclohexane-1-carboxamide (Compound 167)

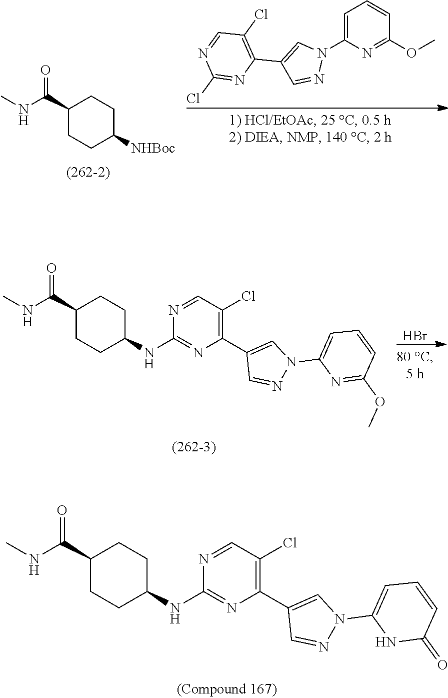

Synthesis of cis-(1s,4s)-4-((5-chloro-4-(1-(6-methoxypyridin-2-yl)-1H-pyrazol-4-yl)pyrimidin-2-yl)amino)-N-methylcyclohexane-1-carboxamide (262-3). A solution of cis-tert-butyl N-[4-(methylcarbamoyl) cyclohexyl]carbamate (262-2) (238.72 mg, 931.25 μmol, 1 eq) in HCl/EtOAc (5 mL) was stirred at 25 °C for 0.5 h. The reaction mixture was concentrated under reduced pressure to give a residue and then the residue in NMP (5 mL) was added 2 2,5-dichloro-4-(1-(6-methoxypyridin-2-yl)-1H-pyrazol-4-yl)pyrimidine (Compound NN)) (0.3 g, 931.25 μmol, 1 eq) and DIEA (601.77 mg, 4.66 mmol, 811.01 μL, 5 eq) at 25 °C. The mixture was stirred at 140 °C for 2 h. LC-MS showed 2,5-dichloro-4-(1-(6-methoxypyridin-2-yl)-1H-pyrazol-4-yl)pyrimidine (Compound NN)) was consumed completely and desired mass was detected. The reaction mixture was partitioned between H₂O 20 mL and EtOAc 20 mL*3. The organic phase was separated, washed with brine 20 mL, dried over Na₂SO₄, filtered and concentrated under reduced pressure to give title compound (262-3) (0.4 g, crude) as a yellow oil. LCMS (ES+): m/z 442.0 [M+H]⁺.

Synthesis of cis-(1s,4s)-4-((5-chloro-4-(1-(6-oxo-1,6-dihydropyridin-2-yl)-1H-pyrazol-4-yl)pyrimidin-2-yl)amino)-N-methylcyclohexane-1-carboxamide (Compound 167). A solution of cis-(1s,4s)-4-((5-chloro-4-(1-(6-methoxypyridin-2-yl)-1H-pyrazol-4-yl)pyrimidin-2-yl)amino)-N-methylcyclohexane-1-carboxamide (262-3) (0.4 g, 905.16 μmol, 1 eq) in HBr (3 mL) was stirred at 80 °C for 5 h. LCMS showed cis-(1s,4s)-4-((5-chloro-4-(1-(6-methoxypyridin-2-yl)-1H-pyrazol-4-yl)pyrimidin-2-yl)amino)-N-methylcyclohexane-1-carboxamide (262-3) was consumed completely and desired mass was detected. The reaction mixture was partitioned between H₂O (10 mL) and EtOAc (5 mL*3). The organic phase was separated, washed with brine (10 mL), dried over Na₂SO₄, filtered and concentrated under reduced pressure to give a residue. The residue was purified by prep-HPLC (Neutral condition; column: Waters xbridge 150*25 mm 10 um; mobile phase: [water (NH₄HCO₃)-ACN]; gradient: 22%-52% B over 14 min) to give title compound (Compound 167) (21.2 mg, 48.01 μmol, 5.30% yield, 96.91% purity) as a white solid. LCMS (ES+): m/z 428.2 [M+H]⁺.

### Example 263. Synthesis of cis-3-((5-chloro-4-(6-(4-fluorophenyl)pyridin-2-yl)pyrimidin-2-yl)amino)-N-methylcyclobutane-1-carboxamide (Compound 115)

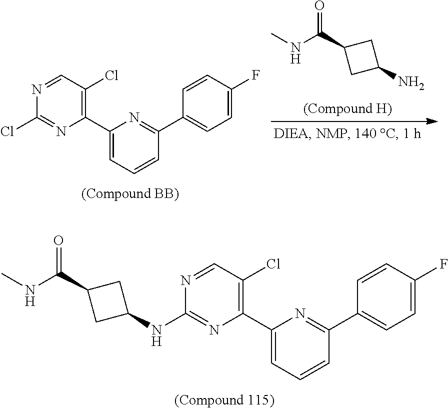

Synthesis of cis-3-((5-chloro-4-(6-(4-fluorophenyl)pyridin-2-yl)pyrimidin-2-yl)amino)-N-methylcyclobutane-1-carboxamide (Compound 115). To a solution of 2,5-dichloro-4-(6-(4-fluorophenyl)pyridin-2-yl)pyrimidine (Compound BB) (0.1 g, 312.36 μmol, 1 eq) in NMP (2 mL) was added DIEA (201.85 mg, 1.56 mmol, 272.03 μL, 5 eq) and cis-3-amino-N-methylcyclobutane-1-carboxamide (Compound H) (40.04 mg, 312.36 μmol, 1 eq). The mixture was stirred at 140 °C for 1 h. LCMS showed 2,5-dichloro-4-(6-(4-fluorophenyl)pyridin-2-yl)pyrimidine (Compound BB) was consumed completely and desired mass was detected. The reaction did not work up, purified directly. The reaction mixture was dissolved in DMF (3 mL), and the resulting solution was purified by prep-HPLC (FA condition, column: Phenomenex luna C18 150*25 mm*10 um; mobile phase: [water(FA)-ACN]; gradient: 42%-72% B over 10 min) to give title compound (Compound 115) (25.4 mg, 60.17 μmol, 19.26% yield, 97.56% purity) as a white solid. LCMS (ES+): m/z 412.1 [M+H]⁺.

### Example 264. Synthesis of cis-3-((5-chloro-4-(1-cyclopropyl-1H-pyrazol-4-yl)pyrimidin-2-yl)amino)-N-methylcyclobutane-1-carboxamide (Compound 97)

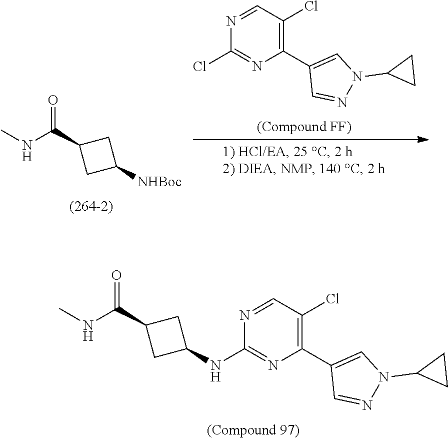 [SMILES: CNC(=O)[C@H]1C[C@@H](NC(=O)OC(C)(C)C)C1 ; Clc1ncc(Cl)c(-c2cnn(C3CC3)c2)n1 ; CNC(=O)[C@H]1C[C@@H](Nc2ncc(Cl)c(-c3cnn(C4CC4)c3)n2)C1]

Synthesis of cis-3-((5-chloro-4-(1-cyclopropyl-1H-pyrazol-4-yl)pyrimidin-2-yl)amino)-N-methylcyclobutane-1-carboxamide (Compound 97). A solution of cis-tert-butyl (3-(methylcarbamoyl)cyclobutyl)carbamate (264-2) (134.23 mg, 588.00 μmol, 1 eq) in HCl/EtOAc (2 mL) was stirred at 25 °C for 2 h. The reaction mixture was concentrated under reduced pressure to give a residue and then the residue in NMP (2 mL) was added 2,5-dichloro-4-(1-cyclopropyl-1H-pyrazol-4-yl)pyrimidine (Compound FF) (0.15 g, 588.00 mol, 1 eq) and DIEA (15.20 mg, 117.60 μmol, 20.48 μL, 0.2 eq) at 25 °C. The mixture was stirred at 140 °C for 2 h. LCMS showed 2,5-dichloro-4-(1-cyclopropyl-1H-pyrazol-4-yl)pyrimidine (Compound FF) was consumed completely and the desired mass was detected. The reaction did not work up, purified directly. The residue was purified by prep-HPLC (neutral condition; column: column: Phenomenex C18 150*25 mm*10 um; mobile phase: [water (NH₄HCO₃)-ACN]; gradient: 26%-56% B over 14 min) to give title compound (Compound 97) (0.032 g, 90.98 μmol, 15.47% yield, 98.6% purity) as a white solid. LCMS (ES+): m/z 347.0 [M+H]⁺.

### Example 265. Synthesis of cis-3-((5-chloro-4-(5-cyclopropyl-1H-pyrazol-3-yl)pyrimidin-2-yl)amino)-N-methylcyclobutane-1-carboxamide (Compound 161)

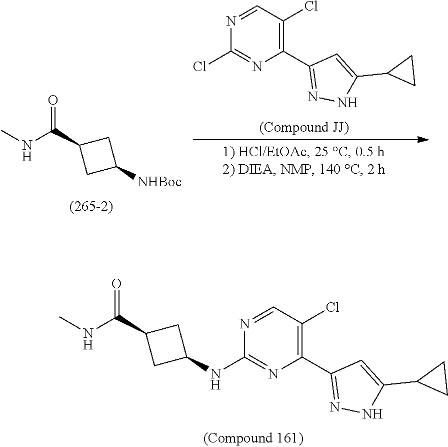 [SMILES: CNC(=O)[C@H]1C[C@@H](NC(=O)OC(C)(C)C)C1 ; Clc1ncc(Cl)c(-c2cc(C3CC3)[nH]n2)n1 ; CNC(=O)[C@H]1C[C@@H](Nc2ncc(Cl)c(-c3cc(C4CC4)[nH]n3)n2)C1]

Synthesis of cis-3-((5-chloro-4-(5-cyclopropyl-1H-pyrazol-3-yl)pyrimidin-2-yl)amino)-N-methylcyclobutane-1-carboxamide (Compound 161). A solution of cis-tert-butyl (3-(methylcarbamoyl)cyclobutyl)carbamate (265-2) (178.98 mg, 784.00 μmol, 1 eq) in HCl/EtOAc (3 mL) was stirred at 25 °C for 0.5 h. The reaction mixture was concentrated under reduced pressure to give a residue and then the residue in NMP (3 mL) was added 2,5-dichloro-4-(5-cyclopropyl-1H-pyrazol-3-yl)pyrimidine (Compound JJ) (0.2 g, 784.00 μmol, 1 eq) and DIEA (101.33 mg, 784.00 μmol, 136.56 μL, 1 eq) at 25 °C the mixture was stirred at 140 °C for 2 h. LCMS showed 2,5-dichloro-4-(5-cyclopropyl-1H-pyrazol-3-yl)pyrimidine (Compound JJ) was consumed completely and the desired mass was detected. The reaction did not work up, purified directly. The residue was purified by prep-HPLC (neutral condition) to give title compound (Compound 161) (0.0395 g, 107.25 μmol, 13.68% yield, 94.17% purity) as a white solid. LCMS (ES+): m/z 347.2 [M+H]⁺.

### Example 266. Synthesis of cis-3-((5-chloro-4-(1-(6-oxo-1,6-dihydropyridin-3-yl)-1H-pyrazol-4-yl)pyrimidin-2-yl)amino)-N-methylcyclobutane-1-carboxamide (Compound 164)

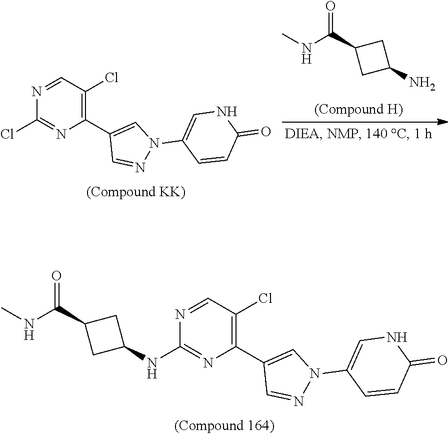

Synthesis of cis-3-((5-chloro-4-(1-(6-oxo-1,6-dihydropyridin-3-yl)-1H-pyrazol-4-yl)pyrimidin-2-yl)amino)-N-methylcyclobutane-1-carboxamide (Compound 164). To a solution of cis-3-amino-N-methylcyclobutane-1-carboxamide (Compound H) (96.18 mg, 584.18 μmol, 1 eq, HCl) in NMP (4 mL) was added DIEA (377.51 mg, 2.92 mmol, 508.77 μL, 5 eq) and 5-(4-(2,5-dichloropyrimidin-4-yl)-1H-pyrazol-1-yl)pyridin-2(5H)-one (Compound KK) (180 mg, 584.18 μmol, 1 eq). The mixture was stirred at 140 °C for 1 h. LCMS showed 5-(4-(2,5-dichloropyrimidin-4-yl)-1H-pyrazol-1-yl)pyridin-2(5H)-one (Compound KK) was consumed completely and 82.7% product with desired mass was detected. The product was dissolved in DMF (2 mL) and filtered to remove the insoluble. The sample was purified by prep-HPLC (FA condition; column: Phenomenex luna C18 150*25 mm*10 um; mobile phase: [water (FA)-ACN]; gradient: 12%-42% B over 10 min) to give title compound (Compound 164) (27 mg, 66.82 μmol, 11.44% yield, 98.95% purity) as a white solid. LCMS (ES+): m/z 400.1[M+H]⁺.

### Example 267. Synthesis of cis-3-((5-chloro-4-(1-(2-oxo-1,2-dihydropyridin-3-yl)-1H-pyrazol-4-yl)pyrimidin-2-yl)amino)-N-methylcyclobutane-1-carboxamide (Compound 193)

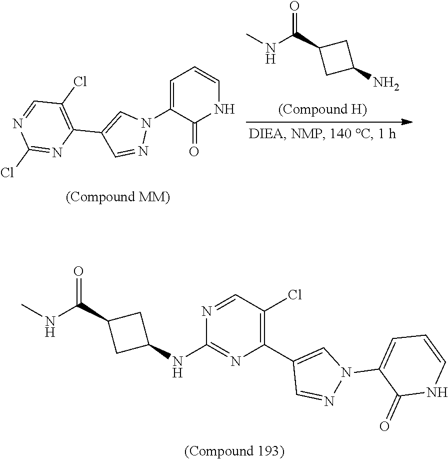

Synthesis of cis-3-((5-chloro-4-(1-(2-oxo-1,2-dihydropyridin-3-yl)-1H-pyrazol-4-yl)pyrimidin-2-yl)amino)-N-methylcyclobutane-1-carboxamide (Compound 193). To a solution of 3-(4-(2,5-dichloropyrimidin-4-yl)-1H-pyrazol-1-yl)pyridin-2(1H)-one (Compound MM) (100 mg, 324.55 μmol, 1 eq) in NMP (2 mL) was added DIEA (209.73 mg, 1.62 mmol, 282.65 μL, 5 eq) and cis-3-amino-N-methylcyclobutane-1-carboxamide (Compound H) (45.76 mg, 277.94 μmol, 8.56e-1 eq, HCl). The mixture was stirred at 140 °C for 2 h. LCMS showed 3-(4-(2,5-dichloropyrimidin-4-yl)-1H-pyrazol-1-yl)pyridin-2(1H)-one (Compound MM) was consumed completely and the desired mass was detected. Vacuum distillation removes solvents to obtain crude products. The crude products were purified by prep-HPLC (column: Welch Xtimate C18 150*25 mm*5 um; mobile phase: [water (FA)-ACN]; gradient: 14%-34% B over 10 min) to give title compound (Compound 193) (5.4 mg, 12.95 μmol, 3.99% yield, 95.91% purity) as a brown solid. LCMS (ES+): m/z 400.2 [M+H]⁺.

### Example 268. Synthesis of cis-(1s,3s)-3-((5-chloro-4-(1-(4-fluorophenyl)-1H-pyrazol-4-yl)pyrimidin-2-yl)amino)-N-methylcyclobutane-1-carboxamide (Compound 122)

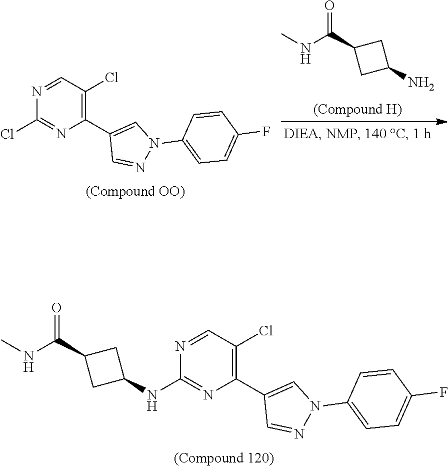

Synthesis of cis-(1s,3s)-3-((5-chloro-4-(1-(4-fluorophenyl)-1H-pyrazol-4-yl)pyrimidin-2-yl)amino)-N-methylcyclobutane-1-carboxamide (Compound 122). To a solution of cis-(1s,3s)-3-amino-N-methylcyclobutane-1-carboxamide (Compound H) (105.79 mg, 642.60 μmol, 1.1 eq, HCl) in NMP (3 mL) was added 2,5-dichloro-4-(1-(4-fluorophenyl)-1H-pyrazol-4-yl)pyrimidine (Compound 00) (180.59 mg, 584.18 μmol, 1 eq) and DIEA (377.51 mg, 2.92 mmol, 508.77 μL, 5 eq). The mixture was stirred at 140 °C for 1 h. LCMS showed 2,5-dichloro-4-(1-(4-fluorophenyl)-1H-pyrazol-4-yl)pyrimidine (Compound 00) was consumed completely and 75.6% peak with desired mass was detected. The reaction did not work up, purified directly. The resulting solution was purified by prep-HPLC (FA condition, column: Welch Xtimate C18 150*25 mm*5 um; mobile phase: [water (FA)-ACN]; gradient: 32%-62% B over 0 min) to give title compound (Compound 122) (85.7 mg, 208.67 μmol, 35.72% yield, 97.60% purity) as a white solid. LCMS (ES+): m/z 401.2 [M+H]⁺.

### Example 269. Synthesis of trans-(1r,3r)-3-((5-chloro-4-(6-(4-fluorophenyl)pyridin-2-yl)pyrimidin-2-yl)amino)-N-methylcyclobutane-1-carboxamide (Compound 140)

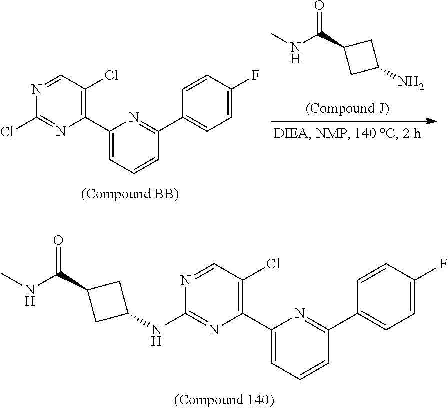

Synthesis of trans-(1r,3r)-3-((5-chloro-4-(6-(4-fluorophenyl)pyridin-2-yl)pyrimidin-2-yl)amino)-N-methylcyclobutane-1-carboxamide (Compound 140). To a solution of 2,5-dichloro-4-(6-(4-fluorophenyl)pyridin-2-yl)pyrimidine (Compound BB) (0.1 g, 312.36 μmol, 1 eq) in NMP (2 mL) was added DIEA (201.85 mg, 1.56 mmol, 272.03 μL, 5 eq) and trans-(1r,3r)-3-amino-N-methylcyclobutane-1-carboxamide (Compound J) (51.42 mg, 312.36 μmol, 1 eq, HCl). The mixture was stirred at 140 °C for 2 h. LCMS showed 2,5-dichloro-4-(6-(4-fluorophenyl)pyridin-2-yl)pyrimidine (Compound BB) was consumed completely and desired mass was detected. The reaction did not work up, purified directly. The reaction mixture was dissolved in DMF (5 mL), and the resulting solution was purified by prep-HPLC (FA condition, column: Phenomenex luna C18 150*25 mm*10 um; mobile phase: [water(FA)-ACN]; gradient: 41%-71% B over 10 min) to give title compound (Compound 140) (16.7 mg, 39.18 μmol, 12.54% yield, 96.63% purity) as a white solid. LCMS (ES+): m/z 412.0 [M+H]⁺.

### Example 270. Synthesis of trans-(1r,3r)-3-((5-chloro-4-(5-cyclopropyl-1H-pyrazol-3-yl)pyrimidin-2-yl)amino)-N-methylcyclobutane-1-carboxamide (Compound 162)

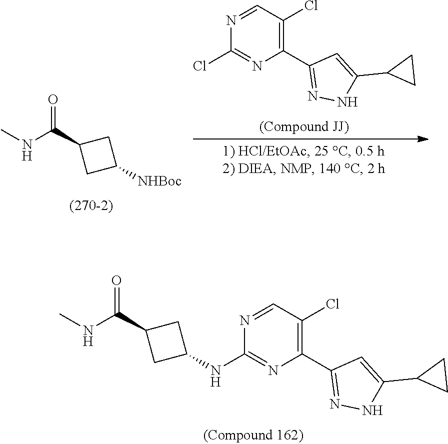 [SMILES: CNC(=O)[C@H]1C[C@H](NC(=O)OC(C)(C)C)C1 ; Clc1ncc(Cl)c(-c2cc(C3CC3)[nH]n2)n1 ; CNC(=O)[C@H]1C[C@H](Nc2ncc(Cl)c(-c3cc(C4CC4)[nH]n3)n2)C1]

Synthesis of trans-(1r,3r)-3-((5-chloro-4-(5-cyclopropyl-1H-pyrazol-3-yl)pyrimidin-2-yl)amino)-N-methylcyclobutane-1-carboxamide (Compound 162). A solution of trans-tert-butyl ((1r,3r)-3-(methylcarbamoyl)cyclobutyl)carbamate (270-2) (178.98 mg, 784.00 μmol, 1 eq) in HCl/EtOAc (3 mL) was stirred at 25 °C for 0.5 h. The reaction mixture was concentrated under reduced pressure to give a residue and then the residue in NMP (3 mL) was added 2,5-dichloro-4-(5-cyclopropyl-1H-pyrazol-3-yl)pyrimidine (Compound JJ) (0.2 g, 784.00 μmol, 1 eq) and DIEA (101.32 mg, 784.00 μmol, 136.55 μL, 1 eq) at 25 °C. The mixture was stirred at 140 °C for 2 h. LCMS showed 2,5-dichloro-4-(5-cyclopropyl-1H-pyrazol-3-yl)pyrimidine (Compound JJ) was consumed completely and the desired mass was detected. The reaction did not work up, purified directly. The residue was purified by prep-HPLC (neutral condition) to give title compound (Compound 162) (45.8 mg, 125.88 μmol, 16.06% yield, 95.32% purity) as a white solid. LCMS (ES+): m/z 347.3 [M+H]⁺.

### Example 271. Synthesis of trans-(1r,3r)-3-((5-chloro-4-(1-(6-oxo-1,6-dihydropyridin-3-yl)-1H-pyrazol-4-yl)pyrimidin-2-yl)amino)-N-methylcyclobutane-1-carboxamide (Compound 119)

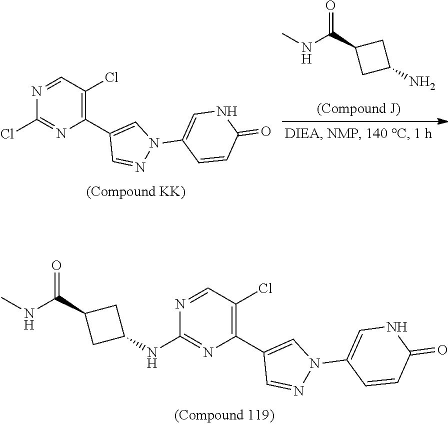

Synthesis of trans-(1r,3r)-3-((5-chloro-4-(1-(6-oxo-1,6-dihydropyridin-3-yl)-1H-pyrazol-4-yl)pyrimidin-2-yl)amino)-N-methylcyclobutane-1-carboxamide (Compound 119). To a solution of trans-(1r,3r)-3-amino-N-methylcyclobutane-1-carboxamide (Compound J) (88.16 mg, 535.50 μmol, 1.1 eq, HCl) in NMP (2 mL) was added DIEA (314.59 mg, 2.43 mmol, 423.98 μL, 5 eq) and 5-(4-(2,5-dichloropyrimidin-4-yl)-1H-pyrazol-1-yl)pyridin-2 (1H)-one (Compound KK) (150.00 mg, 486.82 μmol, 1 eq). The mixture was stirred at 140 °C for 1 h. LCMS showed 5-(4-(2,5-dichloropyrimidin-4-yl)-1H-pyrazol-1-yl)pyridin-2(1H)-one (Compound KK) was consumed completely and 99.4% peak with desired mass was detected. The reaction did not work up, purified directly. The resulting solution was purified by prep-HPLC (FA condition, column: Phenomenex luna C18 150*25 mm*10 um; mobile phase: [water(FA)-ACN]; gradient: 14%-44% B over 10 min) to give title compound (Compound 119) (55.2 mg, 137.74 μmol, 28.29% yield, 99.768% purity) as a white solid. LCMS (ES+): m/z 400.0 [M+H]⁺.

### Example 272. Synthesis of trans-(1r,3r)-3-((5-chloro-4-(1-(2-oxo-1,2-dihydropyridin-4-yl)-1H-pyrazol-4-yl)pyrimidin-2-yl)amino)-N-methylcyclobutane-1-carboxamide (Compound 133)

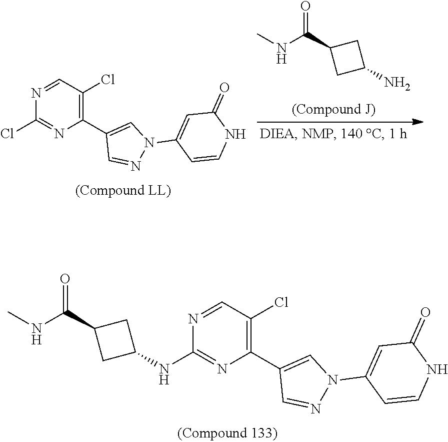

Synthesis of trans-(1r,3r)-3-((5-chloro-4-(1-(2-oxo-1,2-dihydropyridin-4-yl)-1H-pyrazol-4-yl)pyrimidin-2-yl)amino)-N-methylcyclobutane-1-carboxamide (Compound 133). To a solution of trans-(1r,3r)-3-amino-N-methylcyclobutane-1-carboxamide (Compound J) (88.16 mg, 535.50 μmol, 1.1 eq, HCl) in NMP (2 mL) was added DIEA (314.59 mg, 2.43 mmol, 423.98 μL, 5 eq) and 4-(4-(2,5-dichloropyrimidin-4-yl)-1H-pyrazol-1-yl)pyridin-2 (1H)-one (Compound LL) (150 mg, 486.82 μmol, 1 eq). The mixture was stirred at 140 °C for 1 h. LCMS showed 4-(4-(2,5-dichloropyrimidin-4-yl)-1H-pyrazol-1-yl)pyridin-2(1H)-one (Compound LL) was consumed completely and 99.3% peak with desired mass was detected. The reaction did not work up, purified directly. The resulting solution was purified by prep-HPLC (FA condition, column: Phenomenex luna C18 150*25 mm*10 um; mobile phase: [water(FA)-ACN]; gradient: 12%-42% B over 10 min) to give title compound (Compound 133) (18.9 mg, 46.76 μmol, 9.61% yield, 98.92% purity) as a white solid. LCMS (ES+): m/z 399.9 [M+H]⁺.

### Example 273. Synthesis of trans-(1r,3r)-3-((5-chloro-4-(1-(2-oxo-1,2-dihydropyridin-3-yl)-1H-pyrazol-4-yl)pyrimidin-2-yl)amino)-N-methylcyclobutane-1-carboxamide (Compound 166)

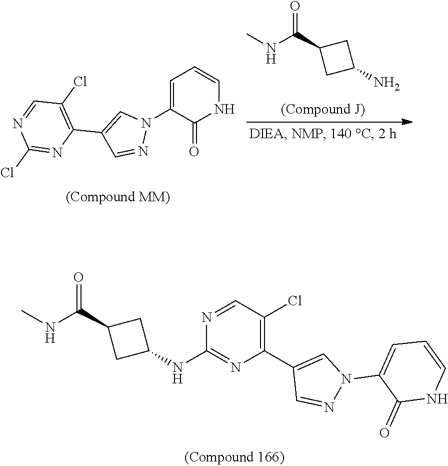

Synthesis of trans-(1r,3r)-3-((5-chloro-4-(1-(2-oxo-1,2-dihydropyridin-3-yl)-1H-pyrazol-4-yl)pyrimidin-2-yl)amino)-N-methylcyclobutane-1-carboxamide (Compound 166). To a solution of 3-(4-(2,5-dichloropyrimidin-4-yl)-1H-pyrazol-1-yl)pyridin-2(1H)-one (Compound MM)) (150 mg, 486.82 μmol, 1 eq) in NMP (2 mL) was added DIEA (314.59 mg, 2.43 mmol, 423.98 μL, 5 eq) and trans-(1r,3r)-3-amino-N-methylcyclobutane-1-carboxamide (Compound J) (88.16 mg, 535.50 μmol, 1.1 eq, HCl). The mixture was stirred at 140 °C for 2 h. LCMS showed 3-(4-(2,5-dichloropyrimidin-4-yl)-1H-pyrazol-1-yl)pyridin-2 (1H)-one (Compound MM)) was consumed completely and the desired mass was detected. After filtration, vacuum distillation removes solvents to obtain crude products. The crude products was purified by prep-HPLC (column: Welch Xtimate C18 150*25 mm*5 um; mobile phase: [water(FA)-ACN]; gradient: 13%-33% B over 9 min) to give title compound (Compound 166) (14.5 mg, 36.27 μmol, 7.45% yield, 100% purity) as a white solid. LCMS (ES⁺): m/z 400.2 [M+H]⁺.

### Example 274. Synthesis trans-(1r,3r)-3-((5-chloro-4-(1-(4-fluorophenyl)-1H-pyrazol-4-yl)pyrimidin-2-yl)amino)-N-methylcyclobutane-1-carboxamide (Compound 123)

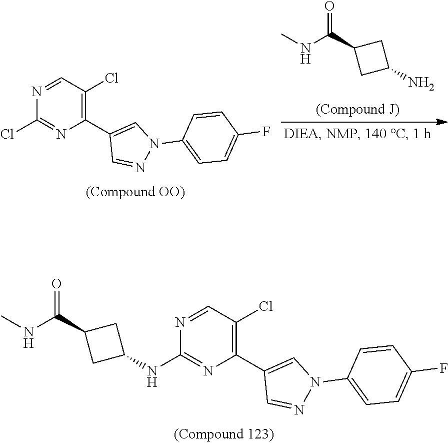

Synthesis of trans-(1r,3r)-3-((5-chloro-4-(1-(4-fluorophenyl)-1H-pyrazol-4-yl)pyrimidin-2-yl)amino)-N-methylcyclobutane-1-carboxamide (Compound 123). To a solution of trans-(1r,3r)-3-amino-N-methylcyclobutane-1-carboxamide (Compound J) (95.86 mg, 582.29 μmol, 1.2 eq, HCl) in NMP (2 mL) was added DIEA (313.57 mg, 2.43 mmol, 422.60 μL, 5 eq) and 2,5-dichloro-4-(1-(4-fluorophenyl)-1H-pyrazol-4-yl)pyrimidine (Compound 00) (150 mg, 485.24 μmol, 1 eq). The mixture was stirred at 140 °C for 1 h. LCMS showed 79.5% of 2,5-dichloro-4-(1-(4-fluorophenyl)-1H-pyrazol-4-yl)pyrimidine (Compound 00) was remaining and 13.9% peak with desired mass was detected. The reaction did not work up, purified directly. The resulting solution was purified by prep-HPLC (FA condition, column: Phenomenex luna C18 150*25 mm*10 um; mobile phase: [water(FA)-ACN]; gradient: 38%-68% B over 10 min) to give title compound (Compound 123) (54 mg, 132.74 μmol, 27.36% yield, 98.53% purity) as a white solid. LCMS (ES+): m/z 401.0 [M+H]⁺.

### Example 275. Synthesis of N-(trans-4-((5-fluoro-4-(3-(6-oxopiperidin-3-yl)phenyl)pyrimidin-2-yl)amino)cyclohexyl)acetamide (Compound 631)

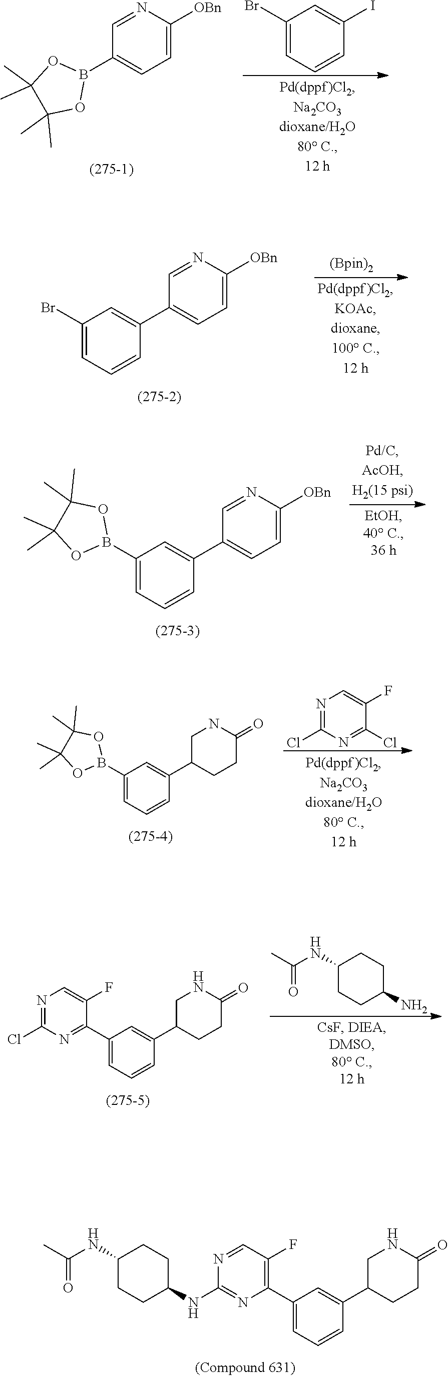

Synthesis of 2-benzyloxy-5-(3-bromophenyl)pyridine (631-2). A mixture of 2-benzyloxy-5-(4,4,5,5-tetramethyl-1,3,2-dioxaborolan-2-yl)pyridine (631-1) (5.00 g, 16.0 mmol, 1.00 eq), 1-bromo-3-iodo-benzene (5.00 g, 17.6 mmol, 2.25 mL, 1.10 eq), Na₂CO₃ (5.11 g, 48.2 mmol, 3.00 eq), Pd(dppf)Cl₂ (1.18 g, 1.61 mmol, 0.100 eq) in dioxane (60 mL) and H₂O (20 mL) was degassed and purged with N₂ for 3 times at 25 °C, and then the mixture was stirred at 80 °C for 12 h under N₂ atmosphere. LC-MS showed the desired mass was detected. The mixture was diluted with water (50 mL), and extracted with EtOAc (50 mL*3). The combined organic layers were washed with brine (20 mL), dried over Na₂SO₄, filtered and concentrated under reduced pressure. The residue was purified by column chromatography (SiO₂, Petroleum ether:Ethyl acetate=1:0 to 10:1) to give the title compound (631-2) (6.00 g, 14.4 mmol, 90.0% yield, 82.0% purity) as a white solid. LCMS (ES⁺): m/z 340.0 [M+H]⁺.

Synthesis of 2-benzyloxy-5-[3-(4,4,5,5-tetramethyl-1,3,2-dioxaborolan-2-yl)phenyl]pyridine (631-3). A mixture of 2-benzyloxy-5-(3-bromophenyl)pyridine (631-2) (3.00 g, 8.82 mmol, 1.00 eq), 4,4,5,5-tetramethyl-2-(4,4,5,5-tetramethyl-1,3,2-dioxaborolan-2-yl)-1,3,2-dioxaborolane (2.46 g, 9.70 mmol, 1.10 eq), Pd(dppf)Cl₂ (645 mg, 881 μmol, 0.100 eq), KOAc (2.60 g, 26.4 mmol, 3.00 eq) in dioxane (50 mL) was degassed and purged with N₂ for 3 times at 25 °C, and then the mixture was stirred at 100 °C for 12 h under N₂ atmosphere. LC-MS showed the desired mass was detected. The mixture was diluted with water (50 mL), and extracted with EtOAc (50 mL*3). The combined organic layers were washed with brine (20 mL), dried over Na₂SO₄, filtered and concentrated under reduced pressure. The residue was purified by column chromatography (SiO₂, Petroleum ether:Ethyl acetate=1:0 to 10:1) to give the title compound (631-3) (2.10 g, 4.61 mmol, 52.2% yield, 85.0% purity) as a brown solid. LCMS (ES⁺): m/z 388.2 [M+H]⁺.

Synthesis of 5-[3-(4,4,5,5-tetramethyl-1,3,2-dioxaborolan-2-yl)phenyl]piperidin-2-one (631-4). To a solution of 2-benzyloxy-5-[3-(4,4,5,5-tetramethyl-1,3,2-dioxaborolan-2-yl)phenyl]pyridine (631-3) (1.00 g, 2.58 mmol, 1.00 eq) in EtOH (20 mL) was added 10% Pd/C (1.00 g, 4.70 mmol, 50.0% purity, 1.82 eq) and AcOH (2.10 g, 34.9 mmol, 2 mL, 13.5 eq) under N₂ atmosphere at 25 °C. The suspension was degassed and purged with H₂ for 3 times. The mixture was stirred under H₂ (15 Psi) at 40 °C for 36 h. LC-MS showed the desired mass was detected. The reaction mixture was filtered and concentrated under reduced pressure. The residue was purified by column chromatography (SiO₂, Petroleum ether:Ethyl acetate=1:0 to 0:1) give the title compound (631-4) (560 mg, 1.12 mmol, 43.2% yield, 60.0% purity) as a white solid. LCMS (ES⁺): m/z 302.1 [M+H]⁺.

Synthesis of 5-[3-(2-chloro-5-fluoro-pyrimidin-4-yl)phenyl]piperidin-2-one (631-5). A mixture of 5-[3-(4,4,5,5-tetramethyl-1,3,2-dioxaborolan-2-yl)phenyl]piperidin-2-one (631-4) (530 mg, 1.76 mmol, 1.00 eq), 2,4-dichloro-5-fluoro-pyrimidine (293 mg, 1.76 mmol, 1.00 eq), Na₂CO₃ (559 mg, 5.28 mmol, 3.00 eq), Pd(dppf)Cl₂ (128 mg, 175 μmol, 0.100 eq) in dioxane (5 mL) and H₂O (2 mL) was degassed and purged with N₂ for 3 times at 25 °C, and then the mixture was stirred at 80 °C for 12 h under N₂ atmosphere. LC-MS showed the desired mass was detected. The mixture was diluted with water (10 mL), and extracted with EtOAc (20 mL*3). The combined organic layers were washed with brine (5 mL), dried over Na₂SO₄, filtered and concentrated under reduced pressure. The residue was purified by column chromatography (SiO₂, Dichloromethane:Methanol=1:0 to 10:1) to give the title compound (631-5) (190 mg, 497 μmol, 28.2% yield, 80.0% purity) as a brown solid. LCMS (ES⁺): m/z 306.1 [M+H]⁺.

Synthesis of N-(trans-4-((5-fluoro-4-(3-(6-oxopiperidin-3-yl)phenyl)pyrimidin-2-yl)amino)cyclohexyl)acetamide (Compound 631). To a solution of 5-[3-(2-chloro-5-fluoro-pyrimidin-4-yl)phenyl]piperidin-2-one (631-5) (140 mg, 457 μmol, 1.00 eq) in DMSO (3 mL) was added CsF (69.5 mg, 457 μmol, 16.9 μL, 1.00 eq), DIEA (177.19 mg, 1.371 mmol, 238.80 μL, 3.00 eq) and N-(trans-4-aminocyclohexyl)acetamide (71.5 mg, 457 μmol, 1.00 eq) at 25 °C. The mixture was stirred at 80 °C for 12 h. LC-MS showed the desired mass was detected. The reaction mixture was filtered and concentrated under reduced pressure. The residue was purified by prep-HPLC (column: Phenomenex Luna C18 100*30 mm*3 um; mobile phase: [1H₂O (0.2% FA)-ACN]; gradient: 10%-50% B over 8.0 min) to give the title compound (Compound 631) (26.1 mg, 61.2 μmol, 13.3% yield, 99.8% purity) as a light orange solid. LCMS (ES⁺): m/z 426.2 [M+H]⁺.

### Example 276. Synthesis of trans-4-((5-fluoro-4-(6-(1-hydroxycyclobutyl)pyridin-2-yl)pyrimidin-2-yl)amino)cyclohexane-1-carboxamide (Compound 620)

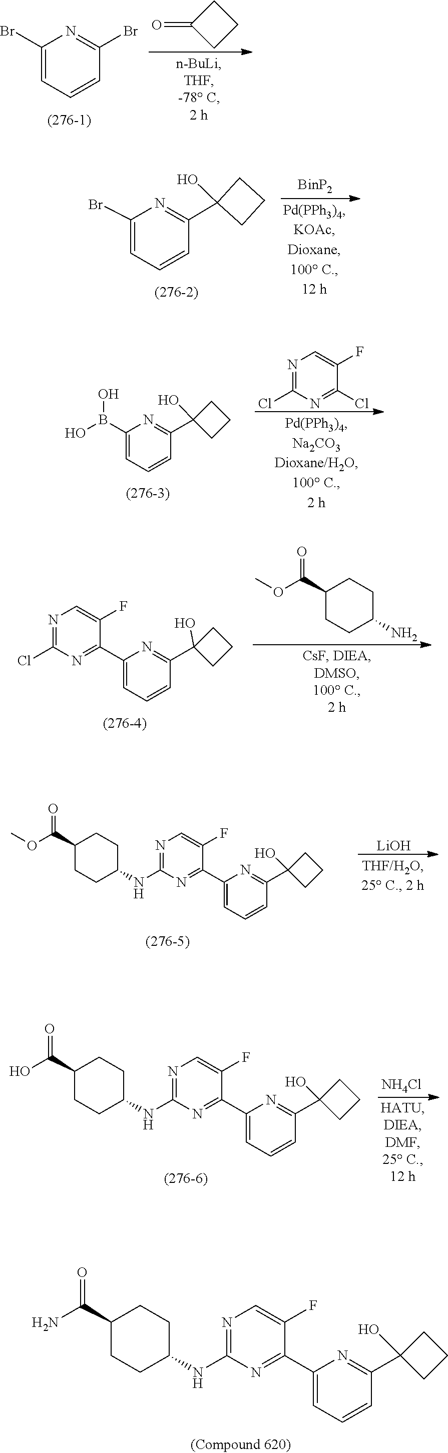

Synthesis of 1-(6-bromopyridin-2-yl)cyclobutan-1-ol (276-2). To a solution of 2,6-dibromopyridine (276-1) (10.0 g, 42.2 mmol, 1.00 eq) in THF (70 mL) was added n-BuLi (2.50 M, 16.8 mL, 1.00 eq) at −78 °C under N₂ atmosphere. The mixture was stirred at −78 °C for 1 h under N₂ atmosphere. Then cyclobutanone (2.96 g, 42.2 mmol, 3.15 mL, 1.00 eq) in THF (40 mL) was added dropwise at −78 °C. The mixture was stirred at −78 °C for 1 h under N₂ atmosphere. LC-MS showed 2,6-dibromopyridine (276-1) was consumed completely and the desired mass was detected. The reaction mixture was quenched by addition sat a.q.NH₄Cl 50 mL at 0 °C, and then extracted with EtOAc (60 mL*3). The combined organic layers were washed with brine (30 mL*3), dried over Na₂SO₄, filtered and concentrated under reduced pressure. The residue was purified by column chromatography (SiO₂, Petroleum ether:Ethyl acetate=1:0 to 5:1) to give the title compound (276-2) (19.0 g, 74.9 mmol, 59.2% yield, 90.0% purity) as a yellow oil. LCMS (ES⁺): m/z 228.0 [M+H]⁺.

Synthesis of (6-(1-hydroxycyclobutyl)pyridin-2-yl)boronic acid (276-3). A mixture of 1-(6-bromopyridin-2-yl)cyclobutan-1-ol (276-2) (7.00 g, 30.6 mmol, 1.00 eq), 4,4,5,5-tetramethyl-2-(4,4,5,5-tetramethyl-1,3,2-dioxaborolan-2-yl)-1,3,2-dioxaborolane (7.79 g, 30.6 mmol, 1.00 eq), Pd(PPh₃)₄ (3.55 g, 3.07 mmol, 0.100 eq), KOAc (9.04 g, 92.0 mmol, 3.00 eq) in dioxane (50 mL) at 25 °C was degassed and purged with N₂ for 3 times, and then the mixture was stirred at 100 °C for 12 h under N₂ atmosphere. LC-MS showed 1-(6-bromopyridin-2-yl)cyclobutan-1-ol (276-2) was consumed completely and the desired mass was detected. The reaction mixture was filtered and concentrated under reduced pressure to give the title compound (8.00 g, crude) as a brown oil. LCMS (ES⁺): m/z 194.2 [M+H]⁺.

Synthesis of 1-(6-(2-chloro-5-fluoropyrimidin-4-yl)pyridin-2-yl)cyclobutan-1-ol (276-4). A mixture of (6-(1-hydroxycyclobutyl)pyridin-2-yl)boronic acid (276-3) (8.00 g, 29.0 mmol, 1.00 eq), 2,4-dichloro-5-fluoro-pyrimidine (4.85 g, 29.0 mmol, 1.00 eq), Na₂CO₃ (4.62 g, 43.6 mmol, 1.50 eq), Pd(PPh₃)₄ (3.36 g, 2.91 mmol, 0.100 eq) in dioxane (20 mL) and H₂O (5 mL) at 25 °C was degassed and purged with N₂ for 3 times, and then the mixture was stirred at 100 °C for 2 h under N₂ atmosphere. LC-MS showed (6-(1-hydroxycyclobutyl)pyridin-2-yl)boronic acid (276-3) was consumed completely and the desired mass was detected. The reaction mixture was quenched by addition H₂O 30 mL at 25 °C, and then combined organic layers were extracted with DCM (70 mL*3), and concentrated under reduced pressure. The residue was purified by column chromatography (SiO₂, Petroleum ether:Ethyl acetate=5:1 to 3:1) to give the title compound (276-4) (2.00 g, 5.69 mmol, 19.5% yield, 79.5% purity) as a yellow solid. LCMS (ES⁺): m/z 280.1 [M+H]⁺.

Synthesis of trans-methyl 4-((5-fluoro-4-(6-(1-hydroxycyclobutyl)pyridin-2-yl)pyrimidin-2-yl)amino)cyclohexane-1-carboxylate (276-5). To a solution of 1-(6-(2-chloro-5-fluoropyrimidin-4-yl)pyridin-2-yl)cyclobutan-1-ol (276-4) (1.90 g, 6.79 mmol, 1.00 eq) in DMSO (20 mL) was added DIEA (2.63 g, 20.3 mmol, 3.55 mL, 3.00 eq), trans-methyl 4-aminocyclohexane-1-carboxylate (1.07 g, 6.79 mmol, 1.00 eq) and CsF (1.03 g, 6.79 mmol, 250 μL, 1.00 eq) at 25 °C. The mixture was stirred at 100 °C for 2 h. LC-MS showed 1-(6-(2-chloro-5-fluoropyrimidin-4-yl)pyridin-2-yl)cyclobutan-1-ol (276-4) was consumed completely and the desired mass was detected. The reaction mixture was quenched by addition H₂O 20 mL at 25 °C, and then diluted with H₂O 10 mL and then extracted with EtOAc (50 mL*3). The combined organic layers were washed with brine (30 mL*3), dried over Na₂SO₄, filtered and concentrated under reduced pressure. The residue was purified by column chromatography (SiO₂, Petroleum ether:Ethyl acetate=1:0 to 1:1) to give the title compound (276-5) (1.00 g, 2.08 mmol, 30.5% yield, 83.1% purity) as a brown oil. LCMS (ES⁺): m/z 401.2 [M+H]⁺.

Synthesis of trans-4-((5-fluoro-4-(6-(1-hydroxycyclobutyl)pyridin-2-yl)pyrimidin-2-yl)amino)cyclohexane-1-carboxylic acid (276-6). To a solution of trans-methyl 4-((5-fluoro-4-(6-(1-hydroxycyclobutyl)pyridin-2-yl)pyrimidin-2-yl)amino)cyclohexane-1-carboxylate (276-5) (400 mg, 998 μmol, 1.00 eq) in THF (5 mL) and H₂O (2 mL) was added LiOH (71.7 mg, 3.00 mmol, 3.00 eq) at 25 °C. The mixture was stirred at 25 °C for 2 h. LC-MS showed trans-methyl 4-((5-fluoro-4-(6-(1-hydroxycyclobutyl)pyridin-2-yl)pyrimidin-2-yl)amino)cyclohexane-1-carboxylate (276-5) was consumed completely and the desired mass was detected. The reaction mixture was quenched by addition H₂O 10 mL at 25 °C, and then diluted with H₂O 10 mL, Adjust PH to 5, and then extracted with EtOAc (30 mL*3). The combined organic layers were washed with brine (30 mL*3), dried over Na₂SO₄, filtered and concentrated under reduced pressure to give the product compound. The product compound was purified by prep-HPLC (column: Phenomenex Luna C18 75*30 mm*3 um; mobile phase: [H₂O (0.2% FA)-ACN]; gradient: 15%-45% B over 8.0 min) to give the title compound (276-6) (45.0 mg, 97.2 μmol, 15.0% yield, 83.5% purity) as a yellow solid. LCMS (ES⁺): m/z 387.2 [M+H]⁺.

Synthesis of trans-4-((5-fluoro-4-(6-(1-hydroxycyclobutyl)pyridin-2-yl)pyrimidin-2-yl)amino)cyclohexane-1-carboxamide (Compound 620). To a solution of trans-4-((5-fluoro-4-(6-(1-hydroxycyclobutyl)pyridin-2-yl)pyrimidin-2-yl)amino)cyclohexane-1-carboxylic acid (276-6) (140 mg, 362 μmol, 1.00 eq) in DMF (3 mL) was added HATU (137 mg, 362 μmol, 1.00 eq), NH₄Cl (96.9 mg, 1.81 mmol, 5.00 eq) and DIEA (140 mg, 1.09 mmol, 189 μL, 3.00 eq) at 25 °C. The mixture was stirred at 25 °C for 12 h. LC-MS showed the desired mass was detected. The reaction mixture was concentrated under reduced pressure. The residue was purified by prep-HPLC (column: Phenomenex Luna C18 100*30 mm*3 um; mobile phase: [H₂O (0.2% FA)-ACN]; gradient: 10%-40% B over 8.0 min) to give the title compound (Compound 620) (33.8 mg, 86.6 μmol, 23.9% yield, 98.8% purity) as a yellow solid. LCMS (ES⁺): m/z 386.2 [M+H]⁺.

### Example 277. Synthesis of trans-N-(4-((5-fluoro-4-(2-oxo-2H-[1,2′-bipyridin]-6′-yl)pyrimidin-2-yl)amino)cyclohexyl)acetamide (Compound 626)

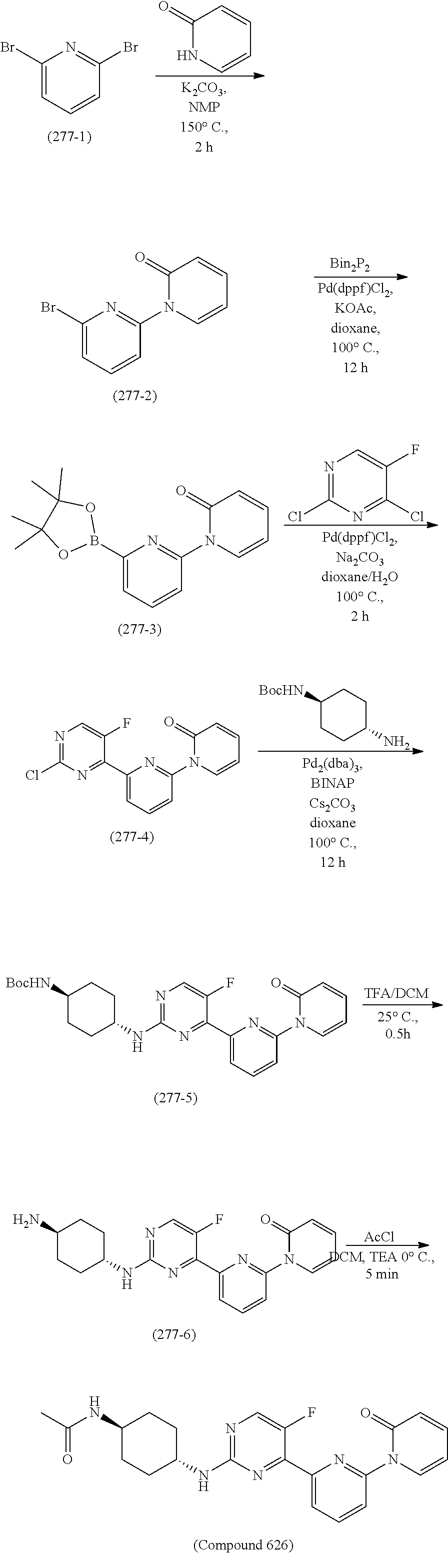

Synthesis of 6′-bromo-2H-[1,2′-bipyridin]-2-one (277-2). To a solution of 2,6-dibromopyridine (277-1) (40.0 g, 169 mmol, 1.00 eq) and pyridin-2(1H)-one (24.0 g, 254 mmol, 1.50 eq) in NMP (1000 mL) was added K₂CO₃ (35.0 g, 254 mmol, 1.50 eq) at 20 °C. The mixture was stirred at 150 °C for 2 h. LCMS showed the desired mass was detected. To the mixture was added H₂O (1000 mL) and the mixture was extracted with EtOAc (1000 mL*3). The combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (277-2) (40.0 g, 148 mmol, 87.8% yield) as a white solid. LCMS (ES⁺): m/z 251.1 [M+H]⁺

Synthesis of 6′-(4,4,5,5-tetramethyl-1,3,2-dioxaborolan-2-yl)-2H-[1,2′-bipyridin]-2-one (277-3). A mixture of 6′-bromo-2H-[1,2′-bipyridin]-2-one (277-2) (15.0 g, 59.8 mmol, 1.00 eq), 4,4,5,5-tetramethyl-2-(4,4,5,5-tetramethyl-1,3,2-dioxaborolan-2-yl)-1,3,2-dioxaborolane (18.2 g, 71.8 mmol, 1.20 eq), KOAc (17.6 g, 179 mmol, 3.00 eq) and Pd(dppf)Cl₂ (4.38 g, 5.98 mmol, 0.100 eq) in dioxane (500 mL) was stirred at 100 °C for 12 h under N₂ atmosphere. LCMS showed 6′-bromo-2H-[1,2′-bipyridin]-2-one (277-2) was consumed and the desired mass was detected. The mixture was concentrated under reduced pressure to give the title compound (277-3) (20.0 g, crude) as a black oil. LCMS (ES⁺): m/z 299.1 [M+H]⁺

Synthesis of 6′-(2-chloro-5-fluoropyrimidin-4-yl)-2H-[1,2′-bipyridin]-2-one (277-4). A mixture of 6′-(4,4,5,5-tetramethyl-1,3,2-dioxaborolan-2-yl)-2H-[1,2′-bipyridin]-2-one (277-3) (15.0 g, 50.4 mmol, 1.00 eq), 2,4-dichloro-5-fluoropyrimidine (8.40 g, 50.4 mmol, 1.00 eq), Na₂CO₃ (10.7 g, 101 mmol, 2.00 eq), Pd(dppf)Cl₂ (1.84 g, 2.52 mmol, 0.0500 eq) in dioxane (300 mL) and H₂O (30 mL) was stirred at 100 °C for 2 h under N₂ atmosphere. LCMS showed 6′-(4,4,5,5-tetramethyl-1,3,2-dioxaborolan-2-yl)-2H-[1,2′-bipyridin]-2-one (277-3) was consumed and the desired mass was detected. To the mixture was added H₂O (300 mL) and the mixture was extracted with EtOAc (300 mL*3). The combined organic layers were dried over Na₂SO₄, filtered and the filtrate was concentrated under reduced pressure to give a residue. The residue was purified by MPLC (SiO₂, PE/EtOAc=1/0 to 5/1) to give the title compound (277-4) (20.0 g, crude) as a yellow oil. LCMS (ES⁺): m/z 303.1 [M+H]⁺

Synthesis of trans-tert-butyl (4-((5-fluoro-4-(2-oxo-2H-[1,2′-bipyridin]-6′-yl)pyrimidin-2-yl)amino)cyclohexyl)carbamate (277-5). To a solution of 6′-(2-chloro-5-fluoropyrimidin-4-yl)-2H-[1,2′-bipyridin]-2-one (277-4) (2.50 g, 8.25 mmol, 1.00 eq) and trans-tert-butyl (4-aminocyclohexyl)carbamate (1.77 g, 8.25 mmol, 1.00 eq) in dioxane (150 mL) were added Cs₂CO₃ (8.05 g, 24.8 mmol, 3.00 eq), BINAP (360 mg, 580 μmol, 0.0700 eq) and Pd₂(dba)₃ (378 mg, 413 μmol, 0.0500 eq) at 20 °C under N₂ atmosphere, the mixture was stirred at 100 °C for 12 h under N₂ atmosphere. LCMS showed 6′-(2-chloro-5-fluoropyrimidin-4-yl)-2H-[1,2′-bipyridin]-2-one (277-4) was consumed and the desired target MS was detected. H₂O (200 mL) was added to the solution and the mixture was extracted with EtOAc (200 mL*3). Then the combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (277-5) (830 mg, 1.73 mmol, 20.9% yield) as a yellow solid. LCMS (ES⁺): m/z 481.2 [M+H]+

Synthesis of trans-6′-(2-((4-aminocyclohexyl)amino)-5-fluoropyrimidin-4-yl)-2H-[1,2′-bipyridin]-2-one (277-6). A solution of trans-tert-butyl (4-((5-fluoro-4-(2-oxo-2H-[1,2′-bipyridin]-6′-yl)pyrimidin-2-yl)amino)cyclohexyl)carbamate (277-5) (121 mg, 252 μmol, 1.00 eq) in DCM (3 mL) and TFA (1 mL) was stirred at 25 °C for 0.5 h. LCMS showed trans-tert-butyl (4-((5-fluoro-4-(2-oxo-2H-[1,2′-bipyridin]-6′-yl)pyrimidin-2-yl)amino)cyclohexyl)carbamate (277-5) was consumed and the desired target MS was detected. The mixture was concentrated under reduced pressure to give the title compound (277-6) (121 mg, crude, TFA) as a yellow oil. LCMS (ES⁺): m/z 381.3 [M+H]⁺

Synthesis of trans-N-(4-((5-fluoro-4-(2-oxo-2H-[1,2′-bipyridin]-6′-yl)pyrimidin-2-yl)amino)cyclohexyl)acetamide (Compound 626). To a solution of trans-6′-(2-((4-aminocyclohexyl)amino)-5-fluoropyrimidin-4-yl)-2H-[1,2′-bipyridin]-2-one (277-1) (200 mg, 405 μmol, 1.00 eq, TFA) in DCM (3 mL) were added TEA (123 mg, 1.21 mmol, 169 μL, 3.00 eq) and acetyl chloride (31.8 mg, 405 μmol, 28.8 μL, 1.00 eq) at 0 °C, the mixture was stirred at 0 °C for 5 min. LCMS showed trans-6′-(2-((4-aminocyclohexyl)amino)-5-fluoropyrimidin-4-yl)-2H-[1,2′-bipyridin]-2-one (277-6) was consumed and the desired target MS was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 10%-50% B over 8.0 min) to give the title compound (Compound 626) (54.9 mg, 130 μmol, 32.1% yield, 99.8% purity) as a pale yellow solid. LCMS (ES+): m/z 423.2 [M+H]⁺.

### Example 278. Synthesis of trans-4-((5-fluoro-4-(2-oxo-2H-[1,2′-bipyridin]-6′-yl)pyrimidin-2-yl)amino)cyclohexane-1-carboxamide (Compound 617)

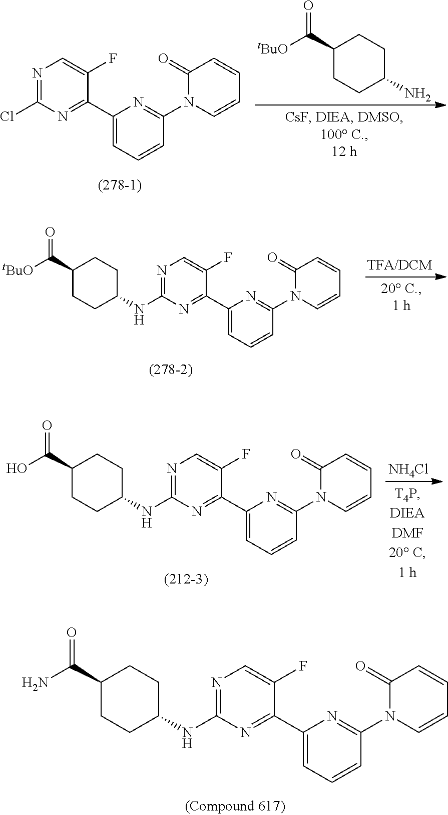

Synthesis of tert-butyl trans-4-((5-fluoro-4-(2-oxo-2H-[1,2′-bipyridin]-6′-yl)pyrimidin-2-yl)amino)cyclohexane-1-carboxylate (278-2). A mixture of 6′-(2-chloro-5-fluoropyrimidin-4-yl)-2H-[1,2′-bipyridin]-2-one (278-1) (2.00 g, 6.61 mmol, 1.00 eq), tert-butyl 4-aminocyclohexanecarboxylate (1.32 g, 6.61 mmol, 1.00 eq), DIEA (2.56 g, 19.8 mmol, 3.45 mL, 3.00 eq), CsF (2.01 g, 13.2 mmol, 487 μL, 2.00 eq) in DMSO (10 mL) at 20 °C was degassed and purged with N₂ for 3 times, and then the mixture was stirred at 100 °C for 12 h under N₂ atmosphere. LC-MS showed the desired mass was detected. The reaction mixture was quenched by addition H₂O 100 mL, and extracted with EtOAc 30 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (278-2) (2.00 g, crude) as a yellow oil. LCMS (ES⁺): m/z 466.1 [M+H]⁺.

Synthesis of trans-4-((5-fluoro-4-(2-oxo-2H-[1,2′-bipyridin]-6′-yl)pyrimidin-2-yl)amino)cyclohexane-1-carboxylic acid (278-3). To a solution of tert-butyl trans-4-((5-fluoro-4-(2-oxo-2H-[1,2′-bipyridin]-6′-yl)pyrimidin-2-yl)amino)cyclohexane-1-carboxylate (278-2) (150 mg, 322 μmol, 1.00 eq) in DCM (3 mL) was added TFA (767 mg, 6.73 mmol, 0.500 mL, 20.8 eq) at 20 °C. The mixture was stirred at 20 °C for 1 h. LC-MS showed the desired mass was detected. The reaction mixture was concentrated under reduced pressure to give the title compound (278-3) (300 mg, crude, TFA) as a yellow oil. LCMS (ES⁺): m/z 410.0 [M+H]⁺.

Synthesis of trans-4-((5-fluoro-4-(2-oxo-2H-[1,2′-bipyridin]-6′-yl)pyrimidin-2-yl)amino)cyclohexane-1-carboxamide (Compound 617). To a solution of trans-4-((5-fluoro-4-(2-oxo-2H-[1,2′-bipyridin]-6′-yl)pyrimidin-2-yl)amino)cyclohexane-1-carboxylic acid (278-3) (300 mg, 732 μmol, 1.00 eq) in DMF (1 mL) was added NH₄Cl (117 mg, 2.20 mmol, 3.00 eq), DIEA (189 mg, 1.47 mmol, 255 μL, 2.00 eq) and T₄P (527 mg, 1.47 mmol, 2.00 eq) at 20 °C. The mixture was stirred at 20 °C for 1 h. LC-MS showed the desired mass was detected. The reaction mixture was quenched by addition H₂O 10 mL, and then diluted with H₂O 10 mL and extracted with EtOAc (10 mL*3). The combined organic layers were washed with sat.aq. NaCl (10 mL), dried over Na₂SO₄, filtered and concentrated under reduced pressure to give a residue. The residue was purified by prep-HPLC (Phenomenex Luna C18 100*30 mm*3 um; mobile phase: [H₂O (0.2% FA)-ACN]; gradient: 1%-40% B over 8.0 min) to give the title compound (Compound 617) (2.1 mg, 4.79 μmol, 6.54e-1% yield, 93.1% purity) as a pale yellow solid. LCMS (ES⁺): m/z 409.1 [M+H]⁺.

### Example 279. Synthesis of cis-3-((5-fluoro-4-(2-(3-oxomorpholino)pyridin-4-yl)pyrimidin-2-yl)amino)cyclohexane-1-carboxamide (Compound 629)

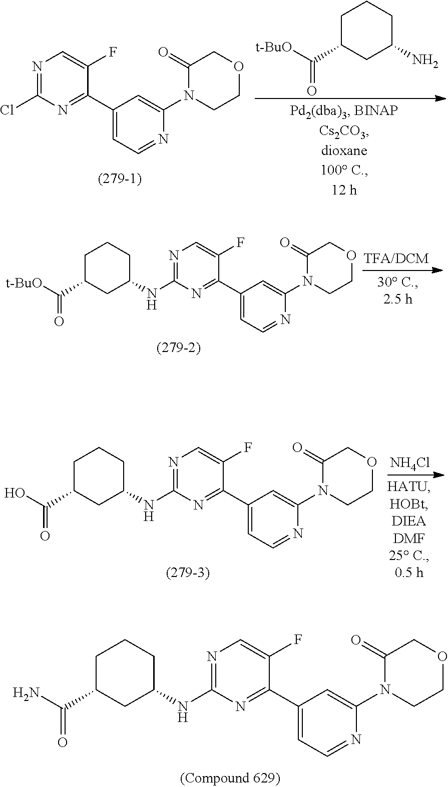

Synthesis of cis-tert-butyl 3-((5-fluoro-4-(2-(3-oxomorpholino)pyridin-4-yl)pyrimidin-2-yl)amino)cyclohexane-1-carboxylate (279-2). To a solution of cis-tert-butyl 3-aminocyclohexane-1-carboxylate (646 mg, 3.24 mmol, 1.00 eq) in dioxane (50 mL) were added 4-(4-(2-chloro-5-fluoropyrimidin-4-yl)pyridin-2-yl)morpholin-3-one (279-1) (1.00 g, 3.24 mmol, 1.00 eq), Cs₂CO₃ (3.17 g, 9.72 mmol, 3.00 eq), BINAP (202 mg, 324 μmol, 0.100 eq) and Pd₂(dba)₃ (295 mg, 324 μmol, 0.100 eq) at 25 °C under N₂ atmosphere. The mixture was stirred at 100 °C for 12 h under N₂ atmosphere. LCMS showed 4-(4-(2-chloro-5-fluoropyrimidin-4-yl)pyridin-2-yl)morpholin-3-one (279-1) was consumed completely and the desired mass was detected. Water (40 mL) was added to the mixture at 25 °C. The mixture was extracted with EtOAc (20 mL*5). The organic layers were dried over anhydrous sodium sulfate, filtered and the filtrate was concentrated under reduced to give a residue. The residue was purified by column chromatography (SiO₂, Petroleum ether:Ethyl acetate=2:1 to 1:1) to give the title compound (279-2) (528 mg, 1.12 mmol, 34.6% yield) as a yellow oil. LCMS (ES⁺): m/z 472.3 [M+H]⁺.

Synthesis of cis-3-((5-fluoro-4-(2-(3-oxomorpholino)pyridin-4-yl)pyrimidin-2-yl)amino)cyclohexane-1-carboxylic acid (279-3). To a solution of cis-tert-butyl 3-((5-fluoro-4-(2-(3-oxomorpholino)pyridin-4-yl)pyrimidin-2-yl)amino)cyclohexane-1-carboxylate (279-2) (300 mg, 636 μmol, 1.00 eq) in DCM (3 mL) was added TFA (1 mL) at 25 °C. The mixture was stirred at 30 °C for 2.5 h. LCMS showed cis-tert-butyl 3-((5-fluoro-4-(2-(3-oxomorpholino)pyridin-4-yl)pyrimidin-2-yl)amino)cyclohexane-1-carboxylate (279-2) was consumed completely and the desired mass was detected. The reaction mixture was concentrated under reduced pressure to give the title compound (279-3) (337 mg, crude, TFA) as a yellow oil. LCMS (ES⁺): m/z 416.2 [M+H]⁺.

Synthesis of cis-3-((5-fluoro-4-(2-(3-oxomorpholino)pyridin-4-yl)pyrimidin-2-yl)amino)cyclohexane-1-carboxamide (Compound 629). To a solution of cis-3-((5-fluoro-4-(2-(3-oxomorpholino)pyridin-4-yl)pyrimidin-2-yl)amino)cyclohexane-1-carboxylic acid (279-3) (337 mg, 636 μmol, 1.00 eq, TFA) in DMF (10 mL) were added HATU (290 mg, 763 μmol, 1.20 eq), HOBt (43.0 mg, 318 μmol, 0.500 eq), DIEA (411 mg, 3.18 mmol, 554 μL, 5.00 eq) and NH₄Cl (136 mg, 2.54 mmol, 4.00 eq) at 25 °C. The mixture was stirred at 25 °C for 0.5 h. LCMS showed cis-3-((5-fluoro-4-(2-(3-oxomorpholino)pyridin-4-yl)pyrimidin-2-yl)amino)cyclohexane-1-carboxylic acid (279-3) was consumed completely and the desired mass was detected. The reaction mixture was concentrated under reduced pressure to give a residue. The residue was purified by prep-HPLC (column: 3_Phenomenex Luna C18 75*30 mm*3 um; mobile phase: [H₂O (0.2% FA)-ACN]; gradient: 10%-40% B over 8.0 min) to give the title compound (Compound 629) (70.2 mg, 168 μmol, 26.4% yield, 99.2% purity) as a yellow solid. LCMS (ES⁺): m/z 415.2 [M+H]⁺.

### Example 280. Synthesis of 4-(4-(2-((1-acetylpiperidin-4-yl)amino)-5-fluoropyrimidin-4-yl)pyridin-2-yl)morpholin-3-one (Compound 628)

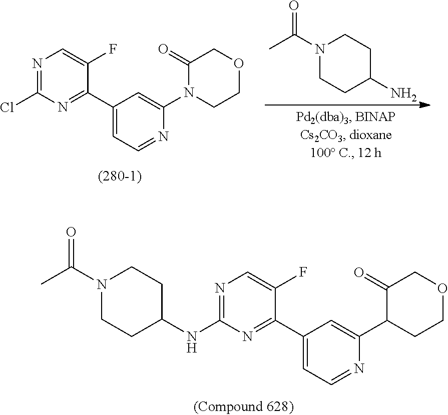

Synthesis of 4-(4-(2-((1-acetylpiperidin-4-yl)amino)-5-fluoropyrimidin-4-yl)pyridin-2-yl)morpholin-3-one (Compound 628). To a solution of 1-(4-aminopiperidin-1-yl)ethan-1-one (138 mg, 972 μmol, 1.00 eq) in dioxane (8 mL) were added 4-(4-(2-chloro-5-fluoropyrimidin-4-yl)pyridin-2-yl)morpholin-3-one (280-1) (300 mg, 972 μmol, 1.00 eq), Cs₂CO₃ (950 mg, 2.92 mmol, 3.00 eq), BINAP (60.5 mg, 97.2 μmol, 0.100 eq) and Pd₂(dba)₃ (89.0 mg, 97.2 μmol, 0.100 eq) at 25 °C under N₂ atmosphere. The mixture was stirred at 100 °C for 12 h. LCMS showed 4-(4-(2-chloro-5-fluoropyrimidin-4-yl)pyridin-2-yl)morpholin-3-one (280-1) was consumed completely and the desired mass was detected. The reaction mixture was filtered with THF (8 mL). The filtrate was concentrated under reduced pressure to give a residue. The residue was purified by prep-HPLC (column: Phenomenex luna C18 100*40 mm*5 um; mobile phase: [H₂O (0.2% FA)-ACN]; gradient: 15%-45% B over 8.0 min) to give the title compound (Compound 628) (32.9 mg, 78.7 μmol, 8.10% yield, 99.2% purity) as a brown gum. LCMS (ES⁺): m/z 415.2 [M+H]⁺.

### Example 281. Synthesis of cis-3-((5-fluoro-4-(6-(3-oxomorpholino)pyridin-2-yl)pyrimidin-2-yl)amino)cyclohexane-1-carboxamide (Compound 627)

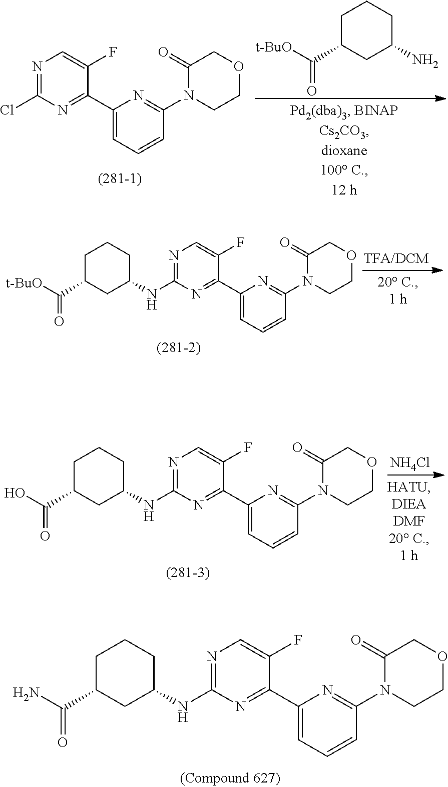

Synthesis of cis-tert-butyl 3-((5-fluoro-4-(6-(3-oxomorpholino)pyridin-2-yl)pyrimidin-2-yl)amino)cyclohexane-1-carboxylate (281-2). To a stirred solution of 4-(6-(2-chloro-5-fluoropyrimidin-4-yl)pyridin-2-yl)morpholin-3-one (281-1) (500 mg, 1.62 mmol, 1.00 eq) and cis-tert-butyl 3-aminocyclohexane-1-carboxylate (484 mg, 2.43 mmol, 1.50 eq) in dioxane (20 mL) were added Cs₂CO₃ (1.06 g, 3.24 mmol, 2.00 eq), BINAP (50.4 mg, 81.0 μmol, 0.0500 eq) and Pd₂(dba)₃ (74.2 mg, 81.0 μmol, 0.0500 eq) at 20 °C under N₂. The resulting mixture was stirred at 100 °C for 12 h under N₂. LCMS showed 4-(6-(2-chloro-5-fluoropyrimidin-4-yl)pyridin-2-yl)morpholin-3-one (281-1) was consumed completely and the desired mass was detected. To the mixture was added H₂O (20 mL) and the mixture was extracted with EtOAc (20 mL*3). The combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (281-2) (400 mg, 848 μmol, 52.4% yield) as a yellow solid. LCMS (ES⁺): m/z 472.2 [M+H]⁺.

Synthesis of cis-3-((5-fluoro-4-(6-(3-oxomorpholino)pyridin-2-yl)pyrimidin-2-yl)amino)cyclohexane-1-carboxylic acid (281-3). A stirred solution of cis-tert-butyl 3-((5-fluoro-4-(6-(3-oxomorpholino)pyridin-2-yl)pyrimidin-2-yl)amino)cyclohexane-1-carboxylate (281-2) (400 mg, 848 μmol, 1.00 eq) in DCM (6 mL) and TFA (2 mL) was stirred at 20 °C for 1 h. LCMS showed cis-tert-butyl 3-((5-fluoro-4-(6-(3-oxomorpholino)pyridin-2-yl)pyrimidin-2-yl)amino)cyclohexane-1-carboxylate (281-2) was consumed completely and the desired mass was detected. The mixture was concentrated under reduced pressure to give the title compound (281-3) (350 mg, crude) as a yellow oil. LCMS (ES⁺): m/z 416.1 [M+H]⁺.

Synthesis of cis-3-((5-fluoro-4-(6-(3-oxomorpholino)pyridin-2-yl)pyrimidin-2-yl)amino)cyclohexane-1-carboxamide (Compound 627). To a stirred solution of cis-3-((5-fluoro-4-(6-(3-oxomorpholino)pyridin-2-yl)pyrimidin-2-yl)amino)cyclohexane-1-carboxylic acid (281-3) (350 mg, 843 μmol, 1.00 eq) and DIEA (327 mg, 2.53 mmol, 440 μL, 3.00 eq) in DMF (8 mL) were added HATU (481 mg, 1.26 mmol, 1.50 eq) and NH₄Cl (90.1 mg, 1.69 mmol, 2.00 eq) at 20 °C. The resulting mixture was stirred at 20 °C for 1 h. LCMS showed cis-3-((5-fluoro-4-(6-(3-oxomorpholino)pyridin-2-yl)pyrimidin-2-yl)amino)cyclohexane-1-carboxylic acid (281-3) was consumed completely and the desired mass was detected. The mixture was concentrated under reduced pressure to give a residue. The residue was purified by prep-HPLC (column: Waters Xbridge Prep OBD C18 150*40 mm*Oum; mobile phase: [H₂O (10 mM NH₄HCO₃)-ACN]; gradient: 15%-45% B over 8.0 min) to give the title compound (Compound 627) (64.1 mg, 153 μmol, 18.2% yield, 99.2% purity) as a white solid. LCMS (ES⁺): m/z 415.2 [M+H]⁺.

### Example 282. Synthesis of 4-(6-(2-((1-acetylpiperidin-4-yl)amino)-5-fluoropyrimidin-4-yl)pyridin-2-yl)morpholin-3-one (Compound 630)

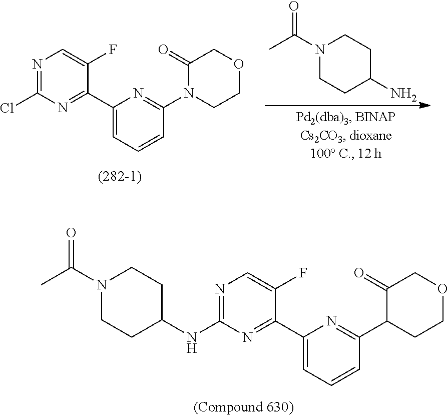

Synthesis of 4-(6-(2-((1-acetylpiperidin-4-yl)amino)-5-fluoropyrimidin-4-yl)pyridin-2-yl)morpholin-3-one (Compound 630). To a solution of 4-(6-(2-chloro-5-fluoropyrimidin-4-yl)pyridin-2-yl)morpholin-3-one (282-1) (300 mg, 972 μmol, 1.00 eq) and 1-(4-aminopiperidin-1-yl)ethan-1-one (276 mg, 1.94 mmol, 2.00 eq) in dioxane (15 mL) were added Pd₂(dba)₃ (89.0 mg, 97.2 μmol, 0.100 eq), BINAP (60.5 mg, 97.2 μmol, 0.100 eq) and Cs₂CO₃ (950 mg, 2.92 mmol, 3.00 eq) at 25 °C under N₂, the mixture was stirred at 100 °C under N₂ for 12 h. LCMS showed 4-(6-(2-chloro-5-fluoropyrimidin-4-yl)pyridin-2-yl)morpholin-3-one (282-1) was consumed completely and the desired mass was detected. The mixture was filtered and the filtrate was concentrated under reduced pressure to give a residue. The residue was purified by prep-HPLC (column: Phenomenex Luna C18 75*30 mm*3 um; mobile phase: [H₂O (0.2% FA)-ACN]; gradient: 20%-50% B over 8.0 min) to give the title compound (Compound 630) (47.8 mg, 111 μmol, 11.4% yield, 95.8% purity) as a yellow solid. LCMS (ES⁺): m/z 415.1 [M+H]⁺.

### Example 283. Synthesis of trans-4-((5-fluoro-4-(6-(2-oxo-1,3-oxazinan-3-yl)pyridin-2-yl)pyrimidin-2-yl)amino)cyclohexane-1-carboxamide (Compound 621)

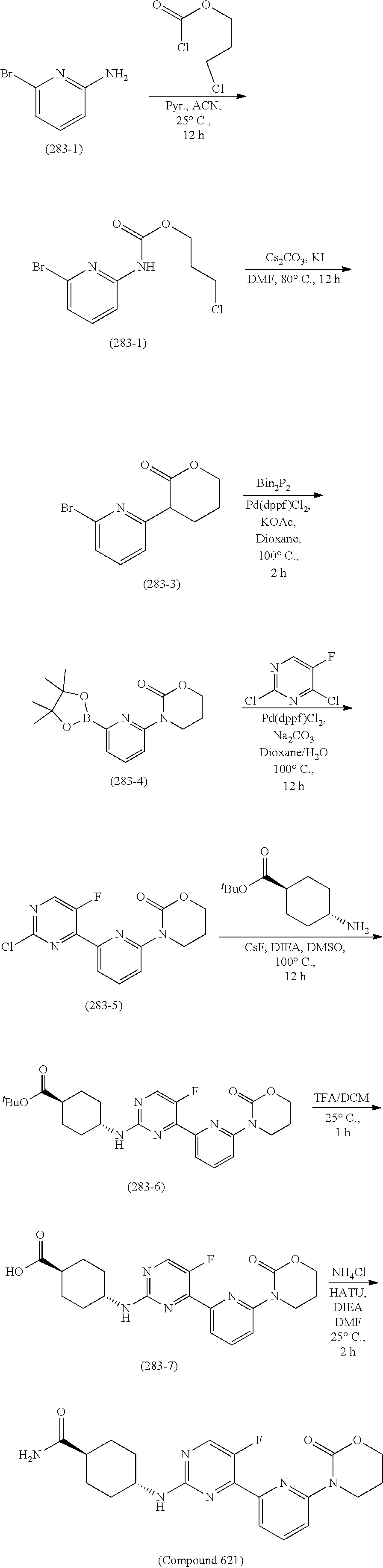

Synthesis of 3-chloropropyl (6-bromopyridin-2-yl)carbamate (283-2). To a solution of 6-bromopyridin-2-amine (283-1) (27.0 g, 156 mmol, 1.00 eq) in ACN (500 mL) was added pyridine (24.6 g, 312 mmol, 25.1 mL, 2.00 eq) and 3-chloropropyl carbonochloridate (24.5 g, 156 mmol, 18.8 mL, 1.00 eq) at 25 °C. The mixture was stirred at 25 °C for 12 h. LC-MS showed the desired mass was detected. The mixture was diluted with water (200 mL), and extracted with EtOAc (300 mL*3). The combined organic layers were washed with brine (100 mL), dried over Na₂SO₄, filtered and concentrated under reduced pressure to give the title compound (283-2) (48.0 g, crude) as a yellow oil. LCMS (ES⁺): m/z 292.9 [M+H]⁺.

Synthesis of 3-(6-bromopyridin-2-yl)-1,3-oxazinan-2-one (283-3). To a solution of 3-chloropropyl (6-bromopyridin-2-yl)carbamate (283-2) (23.0 g, 78.3 mmol, 1.00 eq) in DMF (300 mL) was added Cs₂CO₃ (51.0 g, 156 mmol, 2.00 eq) and KI (26.0 g, 156 mmol, 2.00 eq) at 25 °C. The mixture was stirred at 80 °C for 12 h. LC-MS showed the desired mass was detected. Two batches in parallel were combined and the mixture was diluted with water (1000 mL), and extracted with EtOAc (500 mL*3). The combined organic layers were washed with brine (200 mL), dried over Na₂SO₄, filtered and concentrated under reduced pressure. The residue was purified by column chromatography (SiO₂, Ethyl acetate:Tetrahydrofuran=3:1 to 0:1) to give the title compound (283-3) (40.0 g, 62.2 mmol, 39.7% yield, 40.0% purity) as a yellow oil. LCMS (ES⁺): m/z 257.0 [M+H]⁺

Synthesis of 3-(6-(4,4,5,5-tetramethyl-1,3,2-dioxaborolan-2-yl)pyridin-2-yl)-1,3-oxazinan-2-one (283-4). A mixture of 3-(6-bromopyridin-2-yl)-1,3-oxazinan-2-one (283-3) (20.0 g, 77.8 mmol, 1.00 eq), 4,4,5,5-tetramethyl-2-(4,4,5,5-tetramethyl-1,3,2-dioxaborolan-2-yl)-1,3,2-dioxaborolane (19.7 g, 77.8 mmol, 1.00 eq), Pd(dppf)Cl₂ (5.69 g, 7.78 mmol, 0.100 eq), KOAc (22.9 g, 233 mmol, 3.00 eq) in 1,4-dioxane (100 mL) was degassed and purged with N₂ at 25 °C for 3 times, and then the mixture was stirred at 100 °C for 2 h under N₂ atmosphere. LC-MS showed the desired mass was detected. The crude product (283-4) (20.0 g, 65.7 mmol, 84.5% yield) as a white solid and used into the next step without further purification. LCMS (ES⁺): m/z 223.1 [M+H−82]⁺.

Synthesis of 3-(6-(2-chloro-5-fluoropyrimidin-4-yl)pyridin-2-yl)-1,3-oxazinan-2-one (283-5). A mixture of 3-(6-(4,4,5,5-tetramethyl-1,3,2-dioxaborolan-2-yl)pyridin-2-yl)-1,3-oxazinan-2-one (283-4) (20.0 g, 65.7 mmol, 1.00 eq), 2,4-dichloro-5-fluoro-pyrimidine (10.9 g, 65.7 mmol, 1.00 eq), Na₂CO₃ (20.9 g, 197 mmol, 3.00 eq), Pd(dppf)Cl₂ (4.81 g, 6.58 mmol, 0.100 eq) in 1,4-dioxane (400 mL) and H₂O (80 mL) was degassed and purged with N₂ at 20 °C for 3 times, and then the mixture was stirred at 100 °C for 12 h under N₂ atmosphere. LC-MS showed the desired mass was detected. The reaction mixture was quenched by addition H₂O 100 mL, and then diluted with H₂O 100 mL and extracted with EtOAc (100 mL*3). The combined organic layers were washed with sat.aq. NaCl (100 mL), dried over Na₂SO₄, filtered and concentrated under reduced pressure to give a residue. The residue was purified by column chromatography (SiO₂, Petroleum ether:Ethyl acetate=1:0 to 1:1) to give the title compound (283-5) (10.0 g, 30.2 mmol, 46.0% yield, 93.4% purity) as a yellow solid. LCMS (ES⁺): m/z 309.2 [M+H]⁺.

Synthesis of trans-tert-butyl-4-((5-fluoro-4-(6-(2-oxo-1,3-oxazinan-3-yl)pyridin-2-yl)pyrimidin-2-yl)amino)cyclohexane-1-carboxylate (283-6). A mixture of 3-(6-(2-chloro-5-fluoropyrimidin-4-yl)pyridin-2-yl)-1,3-oxazinan-2-one (283-5) (7.00 g, 22.6 mmol, 1.00 eq), trans-tert-butyl-4-aminocyclohexane-1-carboxylate (4.52 g, 22.6 mmol, 1.00 eq), CsF (6.89 g, 45.3 mmol, 1.67 mL, 2.00 eq), DIEA (5.86 g, 45.3 mmol, 7.90 mL, 2.00 eq) in DMSO (70 mL) was degassed and purged with N₂ at 25 °C for 3 times, and then the mixture was stirred at 100 °C for 12 h under N₂ atmosphere. LC-MS showed the desired mass was detected. The reaction mixture was quenched by addition H₂O 50 mL, and then diluted with H₂O 50 mL and extracted with EtOAc (50 mL*3). The combined organic layers were washed with sat.aq. NaCl (50 mL), dried over Na₂SO₄, filtered and concentrated under reduced pressure. The residue was purified by column chromatography (SiO₂, Petroleum ether:Ethyl acetate=1:0 to 5:1) to give the title compound (283-6) (1.80 g, 3.56 mmol, 23.9% yield, 93.2% purity) as a yellow solid. LCMS (ES⁺): m/z 472.4 [M+H]⁺.

Synthesis of trans-4-((5-fluoro-4-(6-(2-oxo-1,3-oxazinan-3-yl)pyridin-2-yl)pyrimidin-2-yl)amino)cyclohexane-1-carboxylic acid (283-7). To a solution of trans-butyl-4-((5-fluoro-4-(6-(2-oxo-1,3-oxazinan-3-yl)pyridin-2-yl)pyrimidin-2-yl)amino)cyclohexane-1-carboxylate (283-6) (100 mg, 212 μmol, 1.00 eq) in DCM (1 mL) was added TFA (460 mg, 4.04 mmol, 0.3 mL, 19.0 eq) at 25 °C. The mixture was stirred at 25 °C for 1 h. LC-MS showed the desired mass was detected. The reaction mixture was concentrated under reduced pressure to give the title compound (283-7) (100 mg, crude, TFA) as a brown oil. LCMS (ES⁺): m/z 416.2 [M+H]⁺.

Synthesis of trans-4-((5-fluoro-4-(6-(2-oxo-1,3-oxazinan-3-yl)pyridin-2-yl)pyrimidin-2-yl)amino)cyclohexane-1-carboxamide (Compound 621). To a solution of trans-4-((5-fluoro-4-(6-(2-oxo-1,3-oxazinan-3-yl)pyridin-2-yl)pyrimidin-2-yl)amino)cyclohexane-1-carboxylic acid (283-7) (100 mg, 188 μmol, 1.00 eq, TFA) in DMF (3 mL) was added DIEA (73.2 mg, 566 μmol, 98.7 μL, 3.00 eq) until pH-7. Then NH₄Cl (50.5 mg, 944 μmol, 5.00 eq), DIEA (73.2 mg, 566 μmol, 98.7 μL, 3.00 eq) and HATU (107 mg, 283 μmol, 1.50 eq) was added at 25 °C. The mixture was stirred at 25 °C for 2 h. LC-MS showed the desired mass was detected. The reaction mixture was filtered to give a residue. The residue was purified by prep-HPLC (column: 3_Phenomenex Luna C18 75*30 mm*3 um; mobile phase: [1H₂O (0.2% FA)-ACN]; gradient: 10%-40% B over 8.0 min) to give the title compound (Compound 621) (19.8 mg, 47.4 μmol, 25.1% yield, 99.3% purity) as a yellow solid. LCMS (ES⁺): m/z 415.1 [M+H]⁺.

### Example 284. Synthesis of N-(trans-4-((5-fluoro-4-(6-(2-oxo-1,3-oxazinan-3-yl)pyridin-2-yl)pyrimidin-2-yl)amino)cyclohexyl)acetamide (Compound 624)

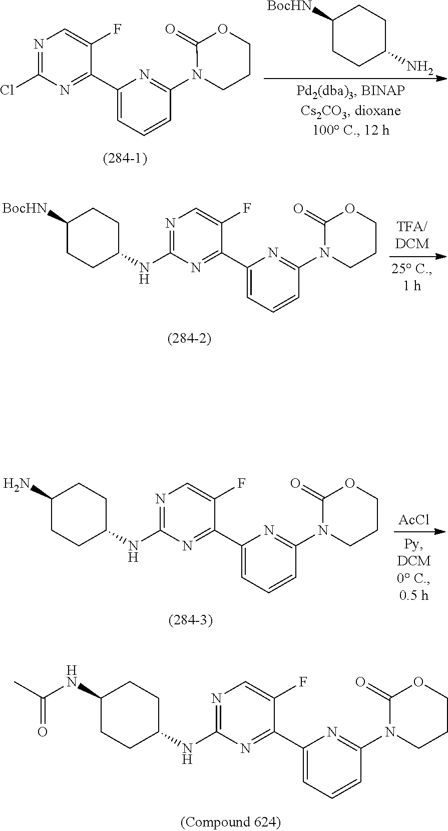

Synthesis of trans-tert-butyl (4-((5-fluoro-4-(6-(2-oxo-1,3-oxazinan-3-yl)pyridin-2-yl)pyrimidin-2-yl)amino)cyclohexyl)carbamate (284-2). To a solution of 3-(6-(2-chloro-5-fluoropyrimidin-4-yl)pyridin-2-yl)-1,3-oxazinan-2-one (284-1) (1.90 g, 6.15 mmol, 1.00 eq) and trans-tert-butyl (4-aminocyclohexyl)carbamate (1.32 g, 6.15 mmol, 1.00 eq) in dioxane (70 mL) were added BINAP (383 mg, 616 μmol, 0.100 eq), Cs₂CO₃ (4.01 g, 12.3 mmol, 2.00 eq) and Pd₂(dba)₃ (564 mg, 616 μmol, 0.100 eq) at 25 °C under N₂ atmosphere. The mixture was stirred at 100 °C for 12 h under N₂ atmosphere. LCMS showed the 3-(6-(2-chloro-5-fluoropyrimidin-4-yl)pyridin-2-yl)-1,3-oxazinan-2-one (284-1) was consumed completely and the desired mass was detected. H₂O (70 mL) was added to the mixture and then the mixture was extracted with EtOAc (50 mL*3). The combined organic layers were washed with brine (50 mL*3), dried over Na₂SO₄, filtered and the filtrate was concentrated under reduced pressure to give a residue. The residue was purified by MPLC (SiO₂, PE/EtOAc=I/O to 1/1) to give the title compound (284-2) (1.03 g, 2.12 mmol, 34.4% yield) as a yellow oil. LCMS (ESI+): m/s 487.2 [M+H]⁺.

Synthesis of trans-3-(6-(2-((4-aminocyclohexyl)amino)-5-fluoropyrimidin-4-yl)pyridin-2-yl)-1,3-oxazinan-2-one (284-3). A solution of trans-tert-butyl (4-((5-fluoro-4-(6-(2-oxo-1,3-oxazinan-3-yl)pyridin-2-yl)pyrimidin-2-yl)amino)cyclohexyl)carbamate (284-2) (120 mg, 247 μmol, 1.00 eq) in DCM (2 mL) and TFA (0.5 mL) was stirred at 25 °C for 1 h. LCMS showed that trans-tert-butyl (4-((5-fluoro-4-(6-(2-oxo-1,3-oxazinan-3-yl)pyridin-2-yl)pyrimidin-2-yl)amino)cyclohexyl)carbamate (284-2) was consumed and the desired mass was detected. The mixture was concentrated under reduced pressure to give the title compound (284-3) (120 mg, crude, TFA) as a yellow oil. LCMS (ESI+): m/s 387.1 [M+H]⁺.

Synthesis of N-(trans-4-((5-fluoro-4-(6-(2-oxo-1,3-oxazinan-3-yl)pyridin-2-yl)pyrimidin-2-yl)amino)cyclohexyl)acetamide (Compound 624). To a solution of 3-(6-(2-((trans-4-aminocyclohexyl)amino)-5-fluoropyrimidin-4-yl)pyridin-2-yl)-1,3-oxazinan-2-one (284-3) (300 mg, 420 μmol, 1.00 eq, TFA) in DCM (4 mL) was added Py (66.4 mg, 839 μmol, 67.7 μL, 2.00 eq) and acetyl chloride (32.9 mg, 420 μmol, 29.8 μL, 1.00 eq) at 0 °C, the mixture was stirred at 0 °C for 0.5 h. LCMS showed that 3-(6-(2-((trans-4-aminocyclohexyl)amino)-5-fluoropyrimidin-4-yl)pyridin-2-yl)-1,3-oxazinan-2-one (284-3) was consumed and the desired mass was detected. The mixture was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 5%-35% B over 8.0 min) to give the title compound (Compound 624) (93.4 mg, 201 μmol, 48.0% yield, 92.4% purity) as a white solid. LCMS (ESI+): m/s 429.2 [M+H]⁺.

### Example 285. Synthesis of trans-N-((1r,4r)-4-((4-(3-cyclopropylphenyl)-5-fluoropyrimidin-2-yl)amino)cyclohexyl)acetamide (Compound 619)

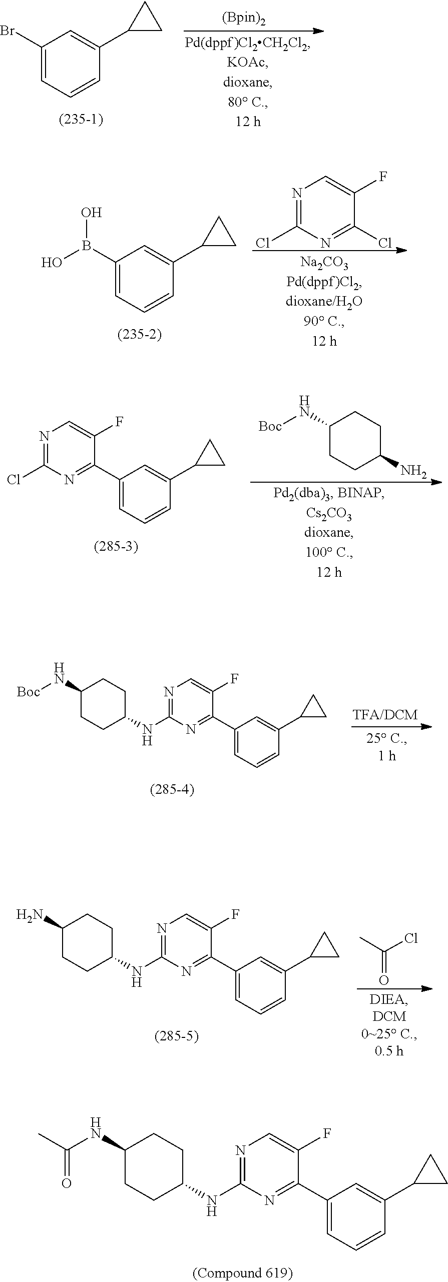

Synthesis of (3-cyclopropylphenyl)boronic acid (285-2). To a solution of 1-bromo-3-cyclopropylbenzene (285-1) (5.00 g, 25.4 mmol, 1.00 eq) in dioxane (50 mL) was added 4,4,5,5-tetramethyl-2-(4,4,5,5-tetramethyl-1,3,2-dioxaborolan-2-yl)-1,3,2-dioxaborolane (19.3 g, 76.1 mmol, 3.00 eq) and KOAc (4.98 g, 50.7 mmol, 2.00 eq), Pd(dppf)Cl₂·CH₂Cl₂ (2.07 g, 2.54 mmol, 0.100 eq) at 20 °C. The mixture was stirred at 80 °C for 12 h under N₂. LCMS showed that one peak with the desired mass was detected. TLC (SiO₂, PE/EtOAc=5/1) indicated 1-bromo-3-cyclopropylbenzene (285-1) was consumed completely and many new spots formed. The crude the title compound (285-2) (5.00 g, 20.5 mmol, 80.7% yield) in dioxane as a black liquid was used into the next step without further purification. LCMS (ES⁺): m/z 161.2 [M−H]⁺.

Synthesis of 2-chloro-4-(3-cyclopropylphenyl)-5-fluoropyrimidine (285-3). To a solution of (3-cyclopropylphenyl)boronic acid (285-2) (4.00 g, 16.4 mmol, 1.00 eq) in dioxane (40 mL) and H₂O (4 mL) were added 2,4-dichloro-5-fluoropyrimidine (4.10 g, 24.6 mmol, 1.50 eq), Pd(dppf)Cl₂ (1.20 g, 1.64 mmol, 0.100 eq) and Na₂CO₃ (3.47 g, 32.8 mmol, 2.00 eq) at 20 °C. The mixture was stirred at 90 °C for 12 h under N₂. LC-MS showed that (3-cyclopropylphenyl)boronic acid (285-2) was consumed completely and one main peak with the desired mass was detected. The mixture was concentrated under reduced pressure. The residue was purified by column chromatography (SiO₂, Petroleum ether/Ethyl acetate=1/0 to 5/1) to give the title compound (285-3) (2.00 g, 8.04 mmol, 49.1% yield, n/a purity) as a colorless oil. LCMS (ES⁺): m/z 249.1 [M+H]⁺.

Synthesis of trans-tert-butyl ((1r,4r)-4-((4-(3-cyclopropylphenyl)-5-fluoropyrimidin-2-yl)amino)cyclohexyl)carbamate (285-4). To a solution of 2-chloro-4-(3-cyclopropylphenyl)-5-fluoropyrimidine (285-3) (2.00 g, 8.04 mmol, 1.00 eq) in dioxane (20 mL) were added trans-tert-butyl ((1r,4r)-4-aminocyclohexyl)carbamate (2.59 g, 12.1 mmol, 1.50 eq) and Pd₂(dba)₃ (736 mg, 804 μmol, 0.100 eq), BINAP (501 mg, 804 μmol, 0.100 eq) and Cs₂CO₃ (5.24 g, 16.1 mmol, 2.00 eq) at 20 °C. The mixture was stirred at 100 °C for 12 h under N₂. LC-MS showed that 2-chloro-4-(3-cyclopropylphenyl)-5-fluoropyrimidine (285-3) was consumed completely and one main peak with the desired mass was detected. The mixture was concentrated under reduced pressure. The residue was purified by column chromatography (SiO₂, Petroleum ether/Ethyl acetate=1/0 to 5/1) to give the title compound (285-4) (650 mg, 1.52 mmol, 19.0% yield, n/a purity) as a yellow solid. LCMS (ES⁺): m/z 427.3 [M+H]⁺.

Synthesis of trans-(1r,4r)-N1-(4-(3-cyclopropylphenyl)-5-fluoropyrimidin-2-yl)cyclohexane-1,4-diamine (285-5). To a solution of trans-tert-butyl ((1r,4r)-4-((4-(3-cyclopropylphenyl)-5-fluoropyrimidin-2-yl)amino)cyclohexyl)carbamate (285-4) (100 mg, 234 μmol, 1.00 eq) in DCM (1 mL) was added TFA (1.54 g, 13.5 mmol, 1 mL, 57.4 eq) at 25 °C. The mixture was stirred at 25 °C for 1 h. TLC (SiO₂, Petroleum ether/Ethyl acetate=1/1) indicated trans-tert-butyl ((1r,4r)-4-((4-(3-cyclopropylphenyl)-5-fluoropyrimidin-2-yl)amino)cyclohexyl)carbamate (285-4) was consumed completely and new spots formed. The mixture was concentrated under reduced pressure to give the title compound (285-5) (70 mg, crude, TFA) as a yellow oil. LCMS (ES⁺): m/z 327.2 [M+H]⁺.

Synthesis of trans-N-((1r,4r)-4-((4-(3-cyclopropylphenyl)-5-fluoropyrimidin-2-yl)amino)cyclohexyl)acetamide (Compound 619). To a mixture of trans-(1r,4r)-N1-(4-(3-cyclopropylphenyl)-5-fluoropyrimidin-2-yl)cyclohexane-1,4-diamine (285-5) (70.0 mg, 159 μmol, 1.00 eq, TFA) and DIEA (103 mg, 795 μmol, 138 μL, 5.00 eq) in DCM (4 mL) was added acetyl chloride (12.5 mg, 159 μmol, 11.3 μL, 1.00 eq) at 0 °C. The reaction was stirred at 25 °C for 0.5 h. LC-MS showed that trans-(1r,4r)-N1-(4-(3-cyclopropylphenyl)-5-fluoropyrimidin-2-yl)cyclohexane-1,4-diamine (285-5) was consumed completely and one main peak with the desired mass was detected. The mixture was concentrated under reduced pressure. The crude product was triturated with EtOAc (2 mL), filtered and the filter cake was dried in a vacuum to give the title compound (Compound 619) (40.4 mg, 105 μmol, 66.3% yield, 96.1% purity) as a white solid. LCMS (ES⁺): m/z 369.2 [M+H]⁺.

### Example 286. Synthesis of trans-(1r,4r)-4-((4-(3-cyclopropylphenyl)-5-fluoropyrimidin-2-yl)amino)cyclohexane-1-carboxamide (Compound 618)

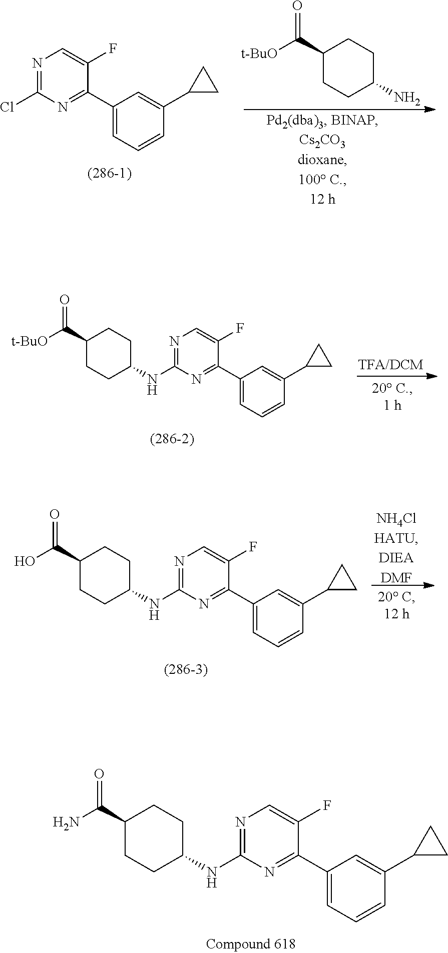 [SMILES: Fc1cnc(Cl)nc1-c1cccc(C2CC2)c1 ; CC(C)(C)OC(=O)[C@H]1CC[C@H](N)CC1 ; CC(C)(C)OC(=O)[C@H]1CC[C@H](Nc2ncc(F)c(-c3cccc(C4CC4)c3)n2)CC1 ; O=C(O)[C@H]1CC[C@H](Nc2ncc(F)c(-c3cccc(C4CC4)c3)n2)CC1 ; "NH4ClHATU,DIEADMF" ; NC(=O)[C@H]1CC[C@H](Nc2ncc(F)c(-c3cccc(C4CC4)c3)n2)CC1]

Synthesis of trans-tert-butyl (1r,4r)-4-((4-(3-cyclopropylphenyl)-5-fluoropyrimidin-2-yl)amino)cyclohexane-1-carboxylate (286-2). To a solution of 2-chloro-4-(3-cyclopropylphenyl)-5-fluoropyrimidine (286-1) (200 mg, 804 μmol, 1.00 eq) and trans-tert-butyl 4-aminocyclohexanecarboxylate (160 mg, 804 μmol, 1.00 eq) in dioxane (3 mL) were added Pd₂(dba)₃ (73.7 mg, 80.4 μmol, 0.100 eq), Cs₂CO₃ (524 mg, 1.61 mmol, 2.00 eq) and BINAP (100 mg, 161 μmol, 0.200 eq) at 20 °C. The mixture was stirred at 100 °C for 12 h under N₂. LC-MS showed that 2-chloro-4-(3-cyclopropylphenyl)-5-fluoropyrimidine (286-1) was consumed completely and one main peak with desired MS was detected. The reaction mixture was filtered and concentrated under reduced pressure to remove solvent. The residue was purified by prep-TLC (SiO₂, Petroleum ether/Ethyl acetate=3/1) to give the title compound (286-2) (280 mg, 680 μmol, 84.6% yield) a yellow solid. LCMS (ES⁺): m/z 412.2 [M+H]⁺.

Synthesis of trans-(1r,4r)-4-((4-(3-cyclopropylphenyl)-5-fluoropyrimidin-2-yl)amino)cyclohexane-1-carboxylic acid (286-3). To a solution of trans-tert-butyl (1r,4r)-4-((4-(3-cyclopropylphenyl)-5-fluoropyrimidin-2-yl)amino)cyclohexane-1-carboxylate (286-2) (70.0 mg, 170 μmol, 1.00 eq) in DCM (1 mL) was added TFA (19.4 mg, 170 μmol, 12.6 μL, 1.00 eq) at 20 °C. The mixture was stirred at 20 °C for 1 h. LC-MS showed that trans-tert-butyl (1r,4r)-4-((4-(3-cyclopropylphenyl)-5-fluoropyrimidin-2-yl)amino)cyclohexane-1-carboxylate (286-2) was consumed completely and desired mass was detected. The reaction mixture was concentrated under reduced pressure to remove solvent to give the title Compound (286-3) (60.0 mg, crude) as a yellow oil. LCMS (ES⁺): m/z 356.1 [M+H]⁺.

Synthesis of trans-(1r,4r)-4-((4-(3-cyclopropylphenyl)-5-fluoropyrimidin-2-yl)amino)cyclohexane-1-carboxamide (Compound 618). To a solution of trans-(1r,4r)-4-((4-(3-cyclopropylphenyl)-5-fluoropyrimidin-2-yl)amino)cyclohexane-1-carboxylic acid (286-3) (60.0 mg, 169 μmol, 1.00 eq) and NH₄Cl (9.03 mg, 169 μmol, 1.00 eq) in DMF (2 mL) were added DIEA (65.5 mg, 506 μmol, 88.2 μL, 3.00 eq) and HATU (160 mg, 422 μmol, 2.50 eq) at 20 °C. The mixture was stirred at 20 °C for 12 h. LC-MS showed that trans-(1r,4r)-4-((4-(3-cyclopropylphenyl)-5-fluoropyrimidin-2-yl)amino)cyclohexane-1-carboxylic acid (286-3) was consumed completely and desired mass was detected. The residue was diluted with H₂O (6 mL) and extracted with EtOAc (3 mL*5). The combined organic layers were washed with brine (6 mL), dried over anhydrous Na₂SO₄, filtered and concentrated under reduced pressure. The residue was purified by prep-HPLC (column: Phenomenex Luna C18 100*30 mm*3 um; mobile phase: [H₂O (0.2% FA)-ACN]; gradient: 25%-65% B over 8.0 min) to give the title Compound (Compound 618) (19.1 mg, 53.1 μmol, 31.4% yield, 98.5% purity) as a yellow solid. LCMS (ES⁺): m/z 355.0 [M+H]⁺.

### Example 287. Synthesis of trans-N-(4-((5-fluoro-4-(3-(piperidin-1-yl)phenyl)pyrimidin-2-yl)amino)cyclohexyl)acetamide (Compound 616)

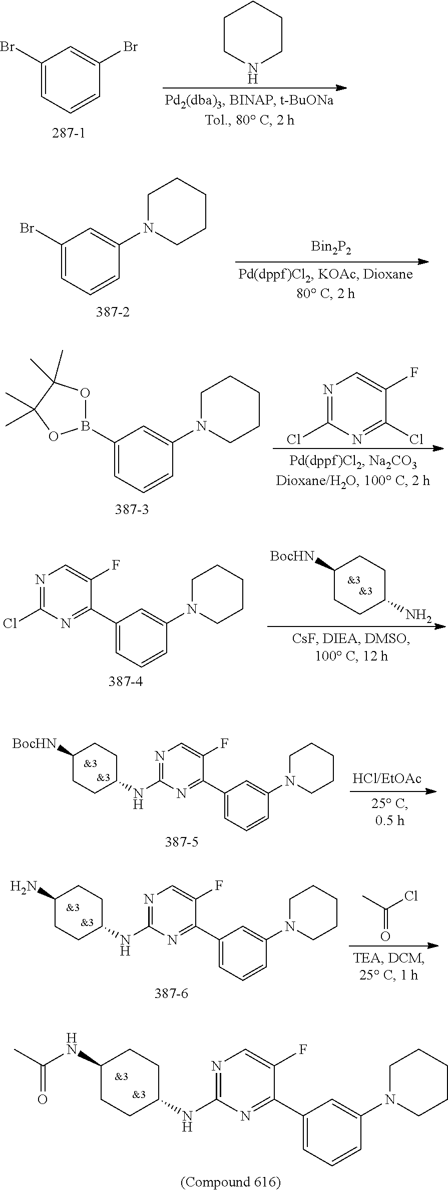

Synthesis of 1-(3-bromophenyl)piperidine (287-2). The reaction was set up in two batches. A mixture of 1,3-dibromobenzene (287-1) (20.0 g, 84.7 mmol, 10.3 mL, 1.00 eq), piperidine (7.22 g, 84.7 mmol, 8.37 mL, 1.00 eq), BINAP (7.92 g, 12.7 mmol, 0.150 eq), Pd₂(dba)₃ (3.88 g, 4.24 mmol, 0.0500 eq) and t-BuONa (16.3 g, 169 mmol, 2.00 eq) in Tol. (100 mL) at 25 °C was degassed and purged with N₂ for 3 times, and then the mixture was stirred at 80 °C for 2 h under N₂ atmosphere. One batch in parallel was monitored in random. LC-MS showed the desired mass was detected. Two in parallel batches were quenched by addition H₂O 80 mL at 25 °C, and then diluted with H₂O 40 mL and then extracted with EtOAc (100 mL*3). The combined organic layers were washed with brine (50 mL*3), dried over Na₂SO₄, filtered and concentrated under reduced pressure. Two in parallel batches were purified by column chromatography (SiO₂, Petroleum ether:Ethyl acetate=5:1 to 3:1) to give the title compound (25.0 g, 70.7 mmol, 41.7% yield, 68.0% purity) as a light yellow oil. LCMS (ES⁺): m/z 240.0 [M+H]⁺.

Synthesis of 1-(3-(4,4,5,5-tetramethyl-1,3,2-dioxaborolan-2-yl)phenyl)piperidine (287-3). A mixture of 1-(3-bromophenyl)piperidine (287-2) (20.0 g, 83.2 mmol, 1.00 eq), 4,4,5,5-tetramethyl-2-(4,4,5,5-tetramethyl-1,3,2-dioxaborolan-2-yl)-1,3,2-dioxaborolane (21.1 g, 83.2 mmol, 1.00 eq), KOAc (24.5 g, 249 mmol, 3.00 eq), Pd(dppf)Cl₂ (6.09 g, 8.33 mmol, 0.100 eq) in dioxane (100 mL) at 25 °C was degassed and purged with N₂ for 3 times, and then the mixture was stirred at 80 °C for 2 h under N₂ atmosphere. LCMS showed the desired mass was detected. The reaction was not processed, directly feed the next step. LCMS (ES⁺): m/z 288.2 [M+H]⁺.

Synthesis of 2-chloro-5-fluoro-4-(3-(piperidin-1-yl)phenyl)pyrimidine (287-4). A mixture of 1-[3-(4,4,5,5-tetramethyl-1,3,2-dioxaborolan-2-yl)phenyl]piperidine (287-3) (20.0 g, 69.6 mmol, 1.00 eq), 2,4-dichloro-5-fluoro-pyrimidine (11.6 g, 69.6 mmol, 1.00 eq), Na₂CO₃ (11.0 g, 104 mmol, 1.50 eq), Pd(dppf)Cl₂ (5.10 g, 6.96 mmol, 0.100 eq) in dioxane (90 mL) and H₂O (20 mL) at 25 °C was degassed and purged with N₂ for 3 times, and then the mixture was stirred at 100 °C for 2 h under N₂ atmosphere. LC-MS showed the desired mass was detected. The reaction was quenched by addition H₂O 80 mL at 25 °C, and then diluted with H₂O 40 mL and then extracted with EtOAc (100 mL*3). The combined organic layers were washed with brine (50 mL*3), dried over Na₂SO₄, filtered and concentrated under reduced pressure. The residue was purified by column chromatography (SiO₂, Petroleum ether:Ethyl acetate=5:1 to 1:1) to give the title compound (20.0 g, 63.3 mmol, 91.0% yield, 92.4% purity) as a yellow solid. LCMS (ES⁺): m/z 292.0 [M+H]⁺.

Synthesis of trans-tert-butyl (4-((5-fluoro-4-(3-(piperidin-1-yl)phenyl)pyrimidin-2-yl)amino)cyclohexyl)carbamate (287-5). To a solution of 2-chloro-5-fluoro-4-[3-(1-piperidyl)phenyl]pyrimidine (287-4) (7.00 g, 23.9 mmol, 1.00 eq) in DMSO (30 mL) was added CsF (3.64 g, 23.9 mmol, 885 μL, 1.00 eq), tert-butyl N-(4-aminocyclohexyl)carbamate (5.14 g, 23.9 mmol, 1.00 eq) and DIEA (9.30 g, 71.9 mmol, 12.5 mL, 3.00 eq) at 25 °C. The mixture was stirred at 100 °C for 12 h. LC-MS showed the desired mass was detected. The reaction mixture was concentrated under reduced pressure. The crude product was purified by re-crystallization from EtOAc (30 mL) at 25 °C to give the title compound (4.50 g, 9.30 mmol, 38.7% yield, 97.0% purity) as a white solid. LCMS (ES⁺): m/z 470.3 [M+H]⁺.

Synthesis of trans-N1-(5-fluoro-4-(3-(piperidin-1-yl)phenyl)pyrimidin-2-yl)cyclohexane-1,4-diamine (287-6). To a solution of trans-tert-butyl (4-((5-fluoro-4-(3-(piperidin-1-yl)phenyl)pyrimidin-2-yl)amino)cyclohexyl)carbamate (287-5) (1.00 g, 2.13 mmol, 1.00 eq) was added HCl/EtOAc (4.00 M, 10.0 mL, 18.7 eq) at 25 °C. The mixture was stirred at 25 °C for 0.5 h. LC-MS showed the desired mass was detected. The reaction mixture was concentrated under reduced pressure to give the title compound (780 mg, crude, HCl) as a white solid. LCMS (ES⁺): m/z 370.2 [M+H]⁺.

Synthesis of trans-N-(4-((5-fluoro-4-(3-(piperidin-1-yl)phenyl)pyrimidin-2-yl)amino)cyclohexyl)acetamide (Compound 616). To a solution of trans-N1-(5-fluoro-4-(3-(piperidin-1-yl)phenyl)pyrimidin-2-yl)cyclohexane-1,4-diamine (287-6) (150 mg, 369.51 μmol, 1.00 eq, HCl) in DCM (5 mL) was added TEA (112 mg, 1.11 mmol, 154 μL, 3.00 eq) and acetyl chloride (23.2 mg, 295 μmol, 21.0 μL, 0.800 eq) at 25 °C. The mixture was stirred at 25 °C for 1 h. LC-MS showed the desired mass was detected. The reaction mixture was concentrated under reduced pressure. The residue was purified by prep-HPLC (column: Phenomenex Luna C18 75*30 mm*3 um; mobile phase: [H₂O (0.2% FA)-ACN]; gradient: 15%-45% B over 8.0 min) to give desired compound as a white solid. The residue was purified by prep-HPLC (column: Phenomenex Luna C18 75*30 mm*3 um; mobile phase: [H₂O (0.2% FA)-ACN]; gradient: 15%-45% B over 8.0 min) to give the title compound (Compound 616) (33.0 mg, 80.1 μmol, 21.7% yield, 100% purity) as a white solid. LCMS (ES⁺): m/z 412.2 [M+H]⁺.

### Example 288. Synthesis of trans-4-((5-fluoro-4-(3-(piperidin-1-yl)phenyl)pyrimidin-2-yl)amino)cyclohexane-1-carboxamide (Compound 615)

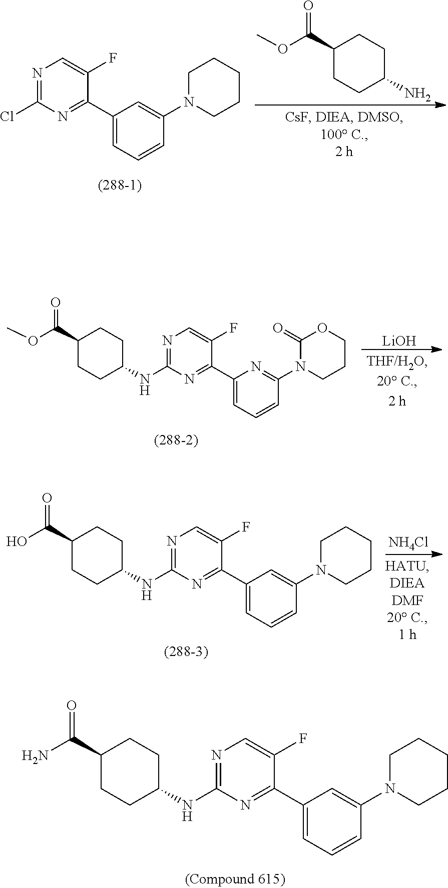

Synthesis of methyl-trans-4-((5-fluoro-4-(3-(piperidin-1-yl)phenyl)pyrimidin-2-yl)amino)cyclohexane-1-carboxylate (288-2). A mixture of 2-chloro-5-fluoro-4-(3-(piperidin-1-yl)phenyl)pyrimidine (288-1)(7.00 g, 23.9 mmol, 1.00 eq), methyl 4-aminocyclohexanecarboxylate (7.54 g, 38.9 mmol, 1.62 eq, HCl), CsF (7.29 g, 47.9 mmol, 1.77 mL, 2.00 eq), DIEA (6.20 g, 47.9 mmol, 8.36 mL, 2.00 eq) in DMSO (70 mL) was degassed and purged with N₂ at 25 °C for 3 times, and then the mixture was stirred at 100 °C for 2 h under N₂ atmosphere. LC-MS showed the desired mass was detected. The solid was filtered, washed with PE (3*50 mL), and dried under high vacuum to give the title compound (288-2) (10 g, crude) as a yellow solid. LCMS (ES⁺): m/z 413.3 [M+H]⁺.

Synthesis of trans-4-((5-fluoro-4-(3-(piperidin-1-yl)phenyl)pyrimidin-2-yl)amino)cyclohexane-1-carboxylic acid (288-3). To a solution of methyl-trans-4-((5-fluoro-4-(3-(piperidin-1-yl)phenyl)pyrimidin-2-yl)amino)cyclohexane-1-carboxylate (288-2) (5.00 g, 12.1 mmol, 1.00 eq) in THF (40 mL) and H₂O (8 mL) was added LiOH (1.45 g, 60.6 mmol, 5.00 eq) at 20 °C. The mixture was stirred at 20 °C for 2 h. LC-MS showed the desired mass was detected. The mixture was adjust PH to 5 with 1M HCl and extracted with EtOAc 100 mL*3 and concentrated under reduced pressure to give the title compound (288-3) (4 g, crude) as a yellow solid. LCMS (ES⁺): m/z 399.3 [M+H]⁺.

Synthesis of trans-4-((5-fluoro-4-(3-(piperidin-1-yl)phenyl)pyrimidin-2-yl)amino)cyclohexane-1-carboxamide (Compound 615). To a solution of trans-4-((5-fluoro-4-(3-(piperidin-1-yl)phenyl)pyrimidin-2-yl)amino)cyclohexane-1-carboxylic acid (288-3) (300 mg, 752 μmol, 1.00 eq) in DMF (3 mL) was added NH₄Cl (120 mg, 2.26 mmol, 3.00 eq), DIEA (194 mg, 1.51 mmol, 262 μL, 2.00 eq) and HATU (572 mg, 1.51 mmol, 2.00 eq) at 20 °C. The mixture was stirred at 20 °C for 1 h. LC-MS showed the desired mass was detected. The reaction mixture was quenched by addition H₂O 10 mL, and then diluted with H₂O 10 mL and extracted with EtOAc (10 mL*3). The combined organic layers were washed with sat.aq. NaCl (10 mL), dried over Na₂SO₄, filtered and concentrated under reduced pressure to give a residue. The residue was purified by prep-HPLC (Phenomenex luna C18 100*40 mm*5 um; mobile phase: [H₂O (0.2% FA)-ACN]; gradient: 1%-40% B over 8.0 min) to give the title compound (Compound 615) (92.6 mg, 231 μmol, 30.6% yield, 99.1% purity) as a pale yellow solid. LCMS (ES⁺): m/z 398.2 [M+H]⁺.

### Example 289. Synthesis of (1R,3S)-3-((4-([1,1′-biphenyl]-3-yl)-5-fluoropyrimidin-2-yl) amino) cyclohexane-1-carboxamide (Compound 612)

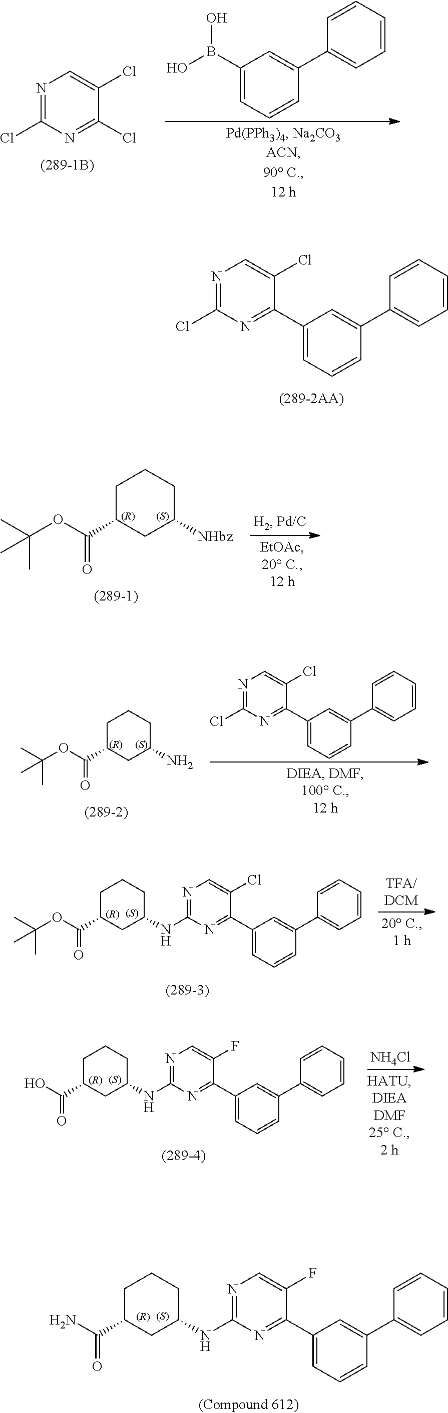

Synthesis of 4-([1,1′-biphenyl]-3-yl)-2,5-dichloropyrimidine. To a solution of 2,4,5-trichloropyrimidine (289-1B) (23.16 g, 126.25 mmol, 1 eq), (3-phenylphenyl)boronic acid (25 g, 126.25 mmol, 1 eq), Pd(PPh₃)₄ (1.46 g, 1.26 mmol, 0.01 eq) and Na₂CO₃ (26.76 g, 252.49 mmol, 2 eq) in CH₃CN (750 mL) was degassed and purged with N₂ for 3 times, and then the mixture was stirred at 90 °C for 12 h under N₂ atmosphere. LCMS showed the desired mass was detected. The reaction mixture was filtered and the filtrate was concentrated under reduced pressure to give a residue. The residue was triturated with MeOH (1 L) at 25 °C for 1 h, then filtered and filter cake was dried under reduced pressure to give the title compound (289-2AA) (75 g, 242.03 mmol, 47.93% yield, 97.190% purity) as a white solid. LCMS (ES⁺): m/z 300.9 [M+H]⁺.

Synthesis of tert-butyl (1R,3S)-3-aminocyclohexane-1-carboxylate (289-2). To a solution of tert-butyl (1R,3S)-3-(((benzyloxy)carbonyl)amino)cyclohexane-1-carboxylate (289-1) (1.00 g, 3.00 mmol, 1.00 eq) in EtOAc (25 mL) was added 10% Pd/C (1.00 g, 50% purity) at 20 °C under H₂ (15 psi) atmosphere. The mixture was stirred at 20 °C for 12 h. LCMS showed tert-butyl (1R,3S)-3-(((benzyloxy)carbonyl)amino)cyclohexane-1-carboxylate (289-1) was consumed completely and the desired mass was detected. The reaction was filtered and the filtrate was concentrated under reduced pressure to give the title compound (289-2) (740 mg, crude) as a colorless oil. LCMS (ES⁺): m/z 200.2 [M+H]⁺.

Synthesis of tert-butyl (1R,3S)-3-((4-([1,1′-biphenyl]-3-yl)-5-chloropyrimidin-2-yl)amino)cyclohexane-1-carboxylate (289-3). To a solution of tert-butyl (1R,3S)-3-aminocyclohexane-1-carboxylate (289-2) (600 mg, 3.01 mmol, 1.00 eq) in DMF (20 mL) were added DIEA (1.17 g, 9.03 mmol, 1.57 mL, 3.00 eq) and 2,5-dichloro-4-(3-phenylphenyl)pyrimidine (907 mg, 3.01 mmol, 1.00 eq) at 20 °C. The mixture was stirred at 100 °C for 12 h. LCMS showed tert-butyl (1R,3S)-3-aminocyclohexane-1-carboxylate (289-2) was consumed completely and the desired mass was detected. Water (20 mL) was added to the mixture at 25 °C. Then the reaction mixture was extracted with EtOAc (15 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=2:1 to 1:1) to give the title compound (289-3) (1.00 g, 2.16 mmol, 71.6% yield) as a yellow oil. LCMS (ES⁺): m/z 464.2 [M+H]⁺.

Synthesis of (1R,3S)-3-((4-([1,1′-biphenyl]-3-yl)-5-chloropyrimidin-2-yl)amino)cyclohexane-1-carboxylic acid (289-4). To a solution of tert-butyl (1R,3S)-3-((4-([1,1′-biphenyl]-3-yl)-5-chloropyrimidin-2-yl)amino)cyclohexane-1-carboxylate (289-3) (300 mg, 647 μmol, 1.00 eq) in DCM (6 mL) was added TFA (2 mL) at 20 °C. The mixture was stirred at 20 °C for 1 h. LCMS showed tert-butyl (1R,3S)-3-((4-([1,1′-biphenyl]-3-yl)-5-chloropyrimidin-2-yl)amino)cyclohexane-1-carboxylate (289-3) was consumed completely and the desired mass was detected. The reaction mixture was concentrated under reduced pressure to give the title compound (289-4) (260 mg, crude) as a yellow oil. LCMS (ES⁺): m/z 408.2 [M+H]⁺.

Synthesis of (1R,3S)-3-((4-([1,1′-biphenyl]-3-yl)-5-fluoropyrimidin-2-yl) amino) cyclohexane-1-carboxamide (Compound 612). To a solution of (1R,3S)-3-((4-([1,1′-biphenyl]-3-yl)-5-fluoropyrimidin-2-yl) amino) cyclohexane-1-carboxylic acid (289-4) (175 mg, 447 μmol, 1.00 eq) in DMF (3 mL) were added HATU (170 mg, 447 μmol, 1.00 eq), DIEA (116 mg, 894 μmol, 156 μL, 2.00 eq) at 25 °C. After addition, NH₄Cl (112 mg, 2.10 mmol, 4.70 eq) was added at 25 °C. The resulting mixture was stirred at 25 °C for 2 h. LCMS showed that (1R,3S)-3-((4-([1,1′-biphenyl]-3-yl)-5-fluoropyrimidin-2-yl) amino) cyclohexane-1-carboxylic acid (289-4) was consumed completely and the desired mass was detected. The mixture was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 40%-70% B over 8.0 min) to give the title compound (Compound 612) (96.7 mg, 247 μmol, 55.3% yield, 99.9% purity) as a white solid. LCMS (ESI+): m/z 391.2 [M+H]⁺.

### Example 290. Synthesis of 1-(4-((4-([1,1′-biphenyl]-3-yl)-5-fluoropyrimidin-2-yl)amino)piperidin-1-yl)ethan-1-one (Compound 611)

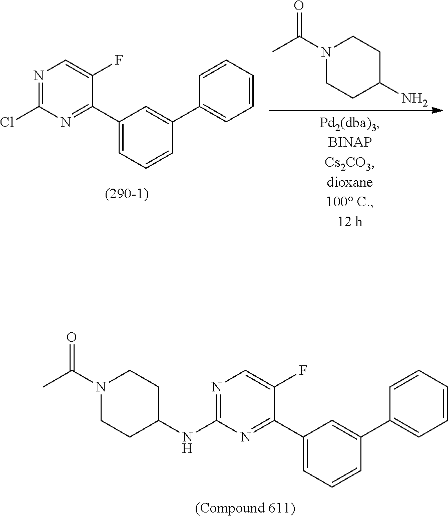

Synthesis of 1-(4-((4-([1,1′-biphenyl]-3-yl)-5-fluoropyrimidin-2-yl)amino)piperidin-1-yl)ethan-1-one (Compound 611). To a solution of 4-([1,1′-biphenyl]-3-yl)-2-chloro-5-fluoropyrimidine (290-1) (500 mg, 1.76 mmol, 1.00 eq) and 1-(4-aminopiperidin-1-yl)ethan-1-one (499 mg, 3.51 mmol, 2.00 eq) in dioxane (20 mL) were added Cs₂CO₃ (1.43 g, 4.39 mmol, 2.50 eq), BINAP (109 mg, 176 μmol, 0.100 eq) and Pd₂(dba)₃ (113 mg, 123 μmol, 0.0700 eq) at 20 °C under N₂ atmosphere. The mixture was stirred at 100 °C for 12 h under N₂ atmosphere. LCMS showed 4-([1,1′-biphenyl]-3-yl)-2-chloro-5-fluoropyrimidine (290-1) was consumed and the desired target MS was detected. H₂O (20 mL) was added to the mixture and the mixture was extracted with EtOAc (20 mL*3). The combined organic layers were washed with brine (10 mL), dried over Na₂SO₄, filtered and the filtrate was concentrated under reduced pressure to give a residue. The residue was purified by prep-HPLC (column: Phenomenex Luna C18 100*30 mm*3 um; mobile phase: [H₂O (0.2% FA)-ACN]; gradient: 40%-75% B over 8.0 min) to give the title compound (Compound 611) (220 mg, 550 μmol, 31.3% yield, 97.9% purity) as a pale yellow solid. LCMS (ES+): m/z 391.2 [M+H]⁺.

### Example 291. Synthesis of trans-4-((4-([1,1′-biphenyl]-3-yl)-5-fluoropyrimidin-2-yl)amino)cyclohexane-1-carboxamide (Compound 610)

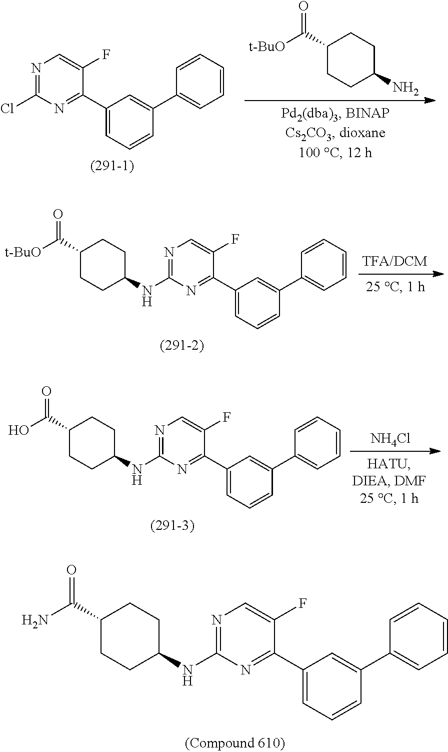 [SMILES: Fc1cnc(Cl)nc1-c1cccc(-c2ccccc2)c1 ; CC(C)(C)OC(=O)[C@H]1CC[C@H](N)CC1 ; CC(C)(C)OC(=O)[C@H]1CC[C@H](Nc2ncc(F)c(-c3cccc(-c4ccccc4)c3)n2)CC1 ; O=C(O)[C@H]1CC[C@H](Nc2ncc(F)c(-c3cccc(-c4ccccc4)c3)n2)CC1 ; "NH4Cl" ; NC(=O)[C@H]1CC[C@H](Nc2ncc(F)c(-c3cccc(-c4ccccc4)c3)n2)CC1]

Synthesis of trans-tert-butyl 4-((4-([1,1′-biphenyl]-3-yl)-5-fluoropyrimidin-2-yl)amino)cyclohexane-1-carboxylate (291-2). To a stirred solution of 4-([1,1′-biphenyl]-3-yl)-2-chloro-5-fluoropyrimidine (291-1) (500 mg, 1.76 mmol, 1.00 eq) and trans-tert-butyl 4-aminocyclohexane-1-carboxylate (700 mg, 3.51 mmol, 2.00 eq) in dioxane (30 mL) were added BINAP (65.6 mg, 105 μmol, 0.0600 eq), Cs₂CO₃ (1.14 g, 3.51 mmol, 2.00 eq) and Pd₂(dba)₃ (48.2 mg, 52.7 μmol, 0.0300 eq) at 25 °C under N₂. The resulting mixture was stirred at 100 °C for 12 h under N₂. LCMS showed 4-([1,1′-biphenyl]-3-yl)-2-chloro-5-fluoropyrimidine (291-1) was consumed completely and the desired mass was detected. To the mixture was added H₂O (30 mL) and the mixture was extracted with EtOAc (20 mL*3). The combined organic layers were dried over Na₂SO₄, filtered and the filtrate was concentrated under reduced pressure to give a residue. The residue was purified by MPLC (SiO₂, PE/EtOAc=1/0 to 1/1) to give the title compound (291-2) (510 mg, 1.14 mmol, 64.9% yield) as a white solid. LCMS (ES+): m/z 448.2 [M+H]⁺.

Synthesis of trans-4-((4-([1,1′-biphenyl]-3-yl)-5-fluoropyrimidin-2-yl)amino)cyclohexane-1-carboxylic acid (291-3). A solution of trans-tert-butyl 4-((4-([1,1′-biphenyl]-3-yl)-5-fluoropyrimidin-2-yl)amino)cyclohexane-1-carboxylate (291-2) (210 mg, 469 μmol, 1.00 eq) in DCM (3 mL) and TFA (1 mL) was stirred at 25 °C for 1 h. LCMS showed trans-tert-butyl 4-((4-([1,1′-biphenyl]-3-yl)-5-fluoropyrimidin-2-yl)amino)cyclohexane-1-carboxylate (291-2) was consumed completely and the desired mass was detected. The mixture was concentrated under reduced pressure to give the title compound (291-3) (183 mg, crude) as a yellow oil. LCMS (ES⁺): m/z 392.1 [M+H]⁺.

Synthesis of trans-4-((4-([1,1′-biphenyl]-3-yl)-5-fluoropyrimidin-2-yl)amino)cyclohexane-1-carboxamide (Compound 610). To a stirred solution of trans-4-((4-([1,1′-biphenyl]-3-yl)-5-fluoropyrimidin-2-yl)amino)cyclohexane-1-carboxylic acid (291-3) (157 mg, 401 μmol, 1.00 eq) and DIEA (156 mg, 1.20 mmol, 210 μL, 3.00 eq) in DMF (4 mL) were added HATU (229 mg, 602 μmol, 1.50 eq) and NH₄Cl (42.9 mg, 802 μmol, 2.00 eq) at 25 °C. The resulting mixture was stirred at 25 °C for 1 h. LCMS showed trans-4-((4-([1,1′-biphenyl]-3-yl)-5-fluoropyrimidin-2-yl)amino)cyclohexane-1-carboxylic acid (291-3) was consumed completely and the desired mass was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 28%-71% B over 8.0 min) to give the title compound (Compound 610) (6.60 mg, 16.8 μmol, 4.19% yield, 99.4% purity) as a white solid. LCMS (ES+): m/z 391.2 [M+H]⁺

### Example 292. Synthesis of trans-N-(4-((4-([1,1′-biphenyl]-3-yl)-5-fluoropyrimidin-2-yl)amino)cyclohexyl)acetamide (Compound 609)

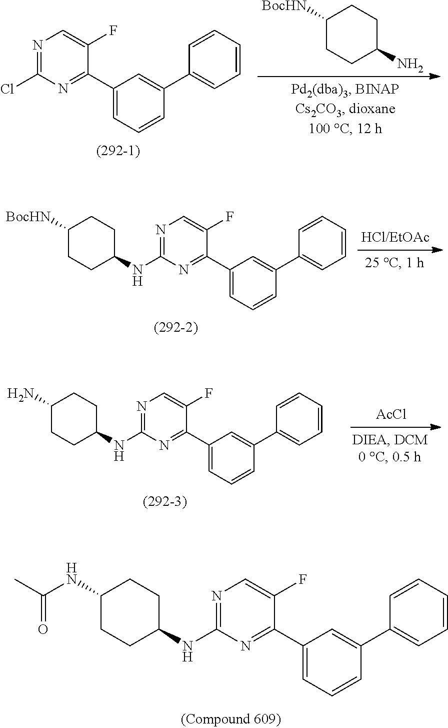 [SMILES: Fc1cnc(Cl)nc1-c1cccc(-c2ccccc2)c1 ; CC(C)(C)OC(=O)N[C@H]1CC[C@H](N)CC1 ; CC(C)(C)OC(=O)N[C@H]1CC[C@H](Nc2ncc(F)c(-c3cccc(-c4ccccc4)c3)n2)CC1 ; N[C@H]1CC[C@H](Nc2ncc(F)c(-c3cccc(-c4ccccc4)c3)n2)CC1 ; "AcCl" ; CC(=O)N[C@H]1CC[C@H](Nc2ncc(F)c(-c3cccc(-c4ccccc4)c3)n2)CC1]

Synthesis of trans-tert-butyl (4-((4-([1,1′-biphenyl]-3-yl)-5-fluoropyrimidin-2-yl)amino)cyclohexyl)carbamate (292-2). To a solution of 4-([1,1′-biphenyl]-3-yl)-2-chloro-5-fluoropyrimidine (292-1) (500 mg, 1.76 mmol, 1.00 eq) and trans-tert-butyl (-aminocyclohexyl)carbamate (753 mg, 3.51 mmol, 2.00 eq) in dioxane (20 mL) were added BINAP (65.6 mg, 105 μmol, 0.0600 eq), Cs₂CO₃ (1.14 g, 3.51 mmol, 2.00 eq) and Pd₂(dba)₃ (48.2 mg, 52.7 μmol, 0.0300 eq) at 25 °C under N₂ atmosphere. The mixture was stirred at 100 °C for 12 h under N₂ atmosphere. LCMS showed 4-([1,1′-biphenyl]-3-yl)-2-chloro-5-fluoropyrimidine (292-1) was consumed and the desired mass was detected. To the mixture was added H₂O (20 mL) and the mixture was extracted with EtOAc (20 mL*3). The combined organic layers were washed with brine (20 mL*3), dried over Na₂SO₄, filtered and the filtrate was concentrated under reduced pressure to give a residue. The residue was purified by MPLC (SiO₂, PE/EtOAc=1/0 to 1/1) to give the title compound (292-2) (750 mg, 1.62 mmol, 92.3% yield) as a yellow solid. LCMS (ES⁺): m/z 463.2 [M+H]+

Synthesis of trans-N1-(4-([1,1′-biphenyl]-3-yl)-5-fluoropyrimidin-2-yl)cyclohexane-1,4-diamine (292-3). A solution of trans-tert-butyl (4-((4-([1,1′-biphenyl]-3-yl)-5-fluoropyrimidin-2-yl)amino)cyclohexyl)carbamate (292-2) (240 mg, 519 μmol, 1.00 eq) in HCl/EtOAc (3 mL) was stirred at 25 °C for 1 h. LCMS showed trans-tert-butyl (4-((4-([1,1′-biphenyl]-3-yl)-5-fluoropyrimidin-2-yl)amino)cyclohexyl)carbamate (292-2) was consumed and the desired mass was detected. The mixture was concentrated under reduced pressure to give the title compound (292-3) (207 mg, crude, HCl) as a yellow oil. LCMS (ES⁺): m/z 363.2 [M+H]⁺.

Synthesis of trans-N-(4-((4-([1,1′-biphenyl]-3-yl)-5-fluoropyrimidin-2-yl)amino)cyclohexyl)acetamide (Compound 609). To a solution of trans-N1-(4-([1,1′-biphenyl]-3-yl)-5-fluoropyrimidin-2-yl)cyclohexane-1,4-diamine (292-3) (207 mg, 519 μmol, 1.00 eq, HCl) in DCM (3 mL) was added DIEA (134 mg, 1.04 mmol, 181 μL, 2.00 eq) and AcCl (32.6 mg, 415 μmol, 29.5 μL, 0.800 eq) at 0 °C. The mixture was stirred at 0 °C for 0.5 h. LCMS showed trans-N1-(4-([1,1′-biphenyl]-3-yl)-5-fluoropyrimidin-2-yl)cyclohexane-1,4-diamine (292-3) was consumed and the desired mass was detected. The mixture was concentrated under reduced pressure to give a residue. The residue was purified by prep-HPLC (column: Phenomenex Luna C18 75*30 mm*3 um; mobile phase: [H₂O (0.2% FA)-ACN]; gradient: 30%-60% B over 8.0 min) to give the title compound (Compound 609) (48.9 mg, 109 μmol, 21.0% yield, 90.2% purity) as a white solid. LCMS (ES⁺): m/z 405.2 [M+H]

### Example 293. Synthesis of trans-N-(4-((5-fluoro-4-(3-morpholinophenyl)pyrimidin-2-yl)amino)cyclohexyl)acetamide (Compound 608)

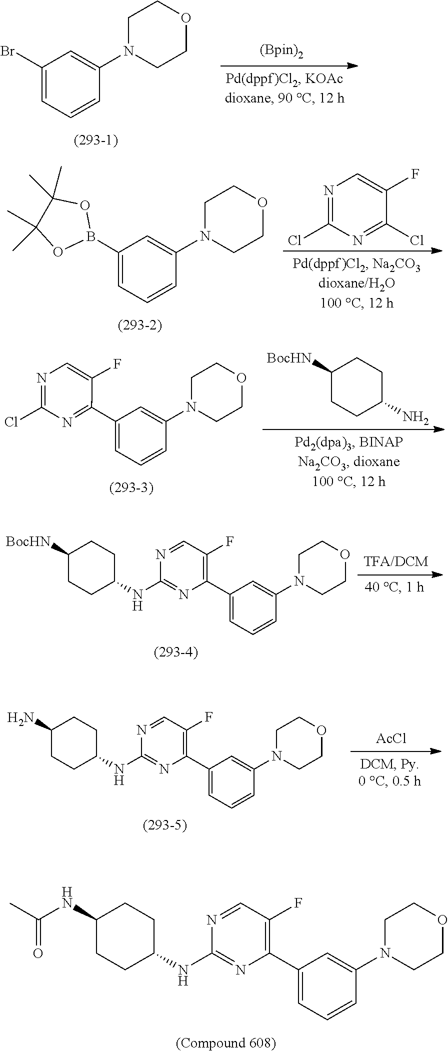

Synthesis of 4-(3-(4,4,5,5-tetramethyl-1,3,2-dioxaborolan-2-yl)phenyl)morpholine (293-2). To a solution of 4-(3-bromophenyl)morpholine (293-1) (20.0 g, 82.6 mmol, 1.00 eq) and 4,4,5,5-tetramethyl-2-(4,4,5,5-tetramethyl-1,3,2-dioxaborolan-2-yl)-1,3,2-dioxaborolane (21.0 g, 82.6 mmol, 1.00 eq) in dioxane (200 mL) were added Pd(dppf)Cl₂ (6.04 g, 8.26 mmol, 0.100 eq) and KOAc (16.2 g, 165 mmol, 2.00 eq) at 25 °C under N₂. After addition, the resulting mixture was stirred at 90 °C for 12 h under N₂ atmosphere. LCMS showed that the desired mass was detected. H₂O (300 mL) was added to the solution, then the mixture was extracted with EtOAc (300 mL*3), the combined organic layers were dried over Na₂SO₄, filtered and the filtrate was concentrated under reduced pressure to give the title compound (293-2) (23.0 g, crude) as a brown solid. LCMS (ES⁺): m/z 290.2 [M+H]⁺.

Synthesis of 4-(3-(2-chloro-5-fluoropyrimidin-4-yl)phenyl)morpholine (293-3). To a solution of 4-(3-(4,4,5,5-tetramethyl-1,3,2-dioxaborolan-2-yl)phenyl)morpholine (293-2) (23.0 g, 79.5 mmol, 1.00 eq) and 2,4-dichloro-5-fluoro-pyrimidine (66.4 g, 398 mmol, 5.00 eq) in dioxane (200 mL) and H₂O (20 mL) were added Na₂CO₃ (16.9 g, 159 mmol, 2.00 eq) and Pd(dppf)Cl₂ (5.82 g, 7.95 mmol, 0.100 eq) at 25 °C under N₂. The resulting mixture was stirred at 100 °C for 12 h under N₂. LCMS showed 4-(3-(4,4,5,5-tetramethyl-1,3,2-dioxaborolan-2-yl)phenyl)morpholine (293-2) was consumed and the desired mass was detected. H₂O (300 mL) was added to the solution, then the mixture was extracted with EtOAc (300 mL*3), the combined organic layers were dried over Na₂SO₄, then concentrated under reduced pressure to give a residue. The residue was purified by MPLC (SiO₂, Petroleum ether/Ethyl acetate=1/0 to 1/1) to give the title compound (293-3) (10.5 g, 35.7 mmol, 44.86% yield) as a yellow solid. LCMS (ES⁺): m/z 294.1 [M+H]⁺.

Synthesis of trans-tert-butyl (4-((5-fluoro-4-(3-morpholinophenyl)pyrimidin-2-yl)amino)cyclohexyl)carbamate (293-4). To a solution of trans-tert-butyl (4-aminocyclohexyl)carbamate (300 mg, 1.40 mmol, 1.00 eq) and 4-[3-(2-chloro-5-fluoro-pyrimidin-4-yl)phenyl]morpholine (293-3) (411 mg, 1.40 mmol, 1.00 eq) in dioxane (8 mL) were added Pd₂(dba)₃ (128 mg, 140 μmol, 0.100 eq), BINAP (87.2 mg, 140 μmol, 0.100 eq) and Cs₂CO₃ (912 mg, 2.80 mmol, 2.00 eq) at 25 °C under N₂, the mixture was stirred at 100 °C for 12 h under N₂. LCMS showed that trans-tert-butyl (4-aminocyclohexyl)carbamate (293-3) was consumed and the desired mass was detected. To the mixture was added H₂O (8 mL) and the mixture was extracted with EtOAc (8 mL*3). The combined organic layers were dried over Na₂SO₄, filtered and the filtrate was concentrated under reduced pressure to give a residue. The residue was purified by prep-TLC (SiO₂, PE/EtOAc=1/1) to give the title compound (293-4) (120 mg, 255 μmol, 18.2% yield) as a yellow solid. LCMS (ES⁺): m/z 472.3 [M+H]⁺.

Synthesis of trans-N-(5-fluoro-4-(3-morpholinophenyl)pyrimidin-2-yl)cyclohexane-1,4-diamine (293-5). To a solution of trans-tert-butyl (4-((5-fluoro-4-(3-morpholinophenyl)pyrimidin-2-yl)amino)cyclohexyl)carbamate (293-4) (120 mg, 255 μmol, 1.00 eq) in DCM (3 mL) was added TFA (1 mL) at 25 °C, the mixture was stirred at 40 °C for 1 h. LCMS showed that trans-tert-butyl (4-((5-fluoro-4-(3-morpholinophenyl)pyrimidin-2-yl)amino)cyclohexyl)carbamate (293-4) was consumed completely and the desired mass was detected. The mixture was concentrated under reduced pressure to give the title compound (293-5) (130 mg, crude, TFA) as a yellow oil liquid. LCMS (ES⁺): m/z 372.3 [M+H]⁺.

Synthesis of trans-N-(4-((5-fluoro-4-(3-morpholinophenyl)pyrimidin-2-yl)amino)cyclohexyl)acetamide (Compound 608). To a solution of trans-N-(5-fluoro-4-(3-morpholinophenyl)pyrimidin-2-yl)cyclohexane-1,4-diamine (293-5) (120 mg, 173 μmol, 1.00 eq, TFA) in DCM (2 mL) were added Py (13.7 mg, 173 μmol, 14.0 μL, 1.00 eq) and acetyl chloride (13.6 mg, 173 μmol, 12.3 μL, 1.00 eq) at 0 °C, the mixture was stirred at 0 °C for 0.5 h. LCMS showed that the trans-N-(5-fluoro-4-(3-morpholinophenyl)pyrimidin-2-yl)cyclohexane-1,4-diamine (293-5) was consumed and the desired mass was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 20%-55% B over 8.0 min) to give the title compound (Compound 608) (14.4 mg, 34.8 μmol, 20.1% yield, 100% purity) as a white solid. LCMS (ES⁺): m/z 414.2 [M+H]⁺

### Example 294. Synthesis of trans-4-((5-fluoro-4-(3-morpholinophenyl) pyrimidin-2-yl) amino) cyclohexane-1-carboxamide (Compound 607)

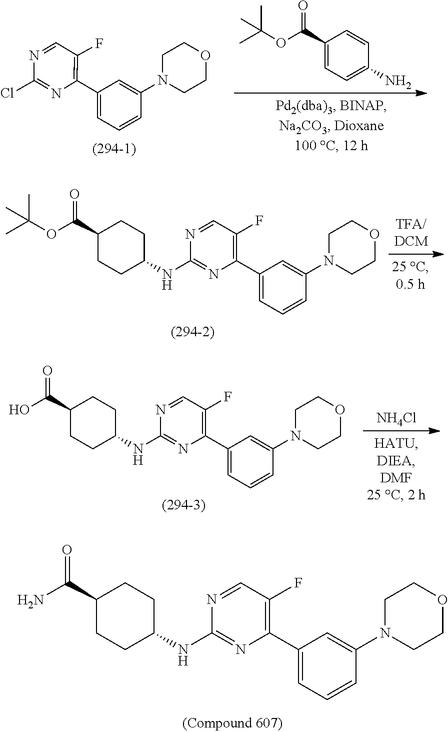

Synthesis of tert-butyl-trans-4-((5-fluoro-4-(3-morpholinophenyl) pyrimidin-2-yl) amino) cyclohexane-1-carboxylate (294-2). To a solution of 4-(3-(2-chloro-5-fluoropyrimidin-4-yl) phenyl) morpholine (294-1) (1.00 g, 3.40 mmol, 1.00 eq) and trans-tert-butyl 4-aminocyclohexane-1-carboxylate (1.02 g, 5.11 mmol, 1.50 eq) in dioxane (30 mL) were added Pd₂ (dba)₃ (312 mg, 340 μmol, 0.100 eq), BINAP (212 mg, 340 μmol, 0.100 eq) and Na₂CO₃ (722 mg, 6.81 mmol, 2.00 eq) at 25 °C under N₂. The resulting mixture was stirred at 100 °C for 12 h under N₂. LCMS showed that 4-(3-(2-chloro-5-fluoropyrimidin-4-yl) phenyl) morpholine (294-1) was consumed completely and the desired mass was detected. H₂O (30 mL) was added to the mixture, then the mixture was extracted with DCM (30 mL*3), the combined organic layers were dried over Na₂SO₄, filtered and the filtrate was concentrated under reduced pressure to give a residue. The residue was purified by MPLC (SiO₂ Petroleum ether/Ethyl acetate=1/0 to 1/1) to give the title compound (294-1) (550 mg, 1.20 mmol, 35.4% yield) as a yellow solid. LCMS (ES⁺): m/z 457.3 [M+H]⁺.

Synthesis of trans-4-((5-fluoro-4-(3-morpholinophenyl) pyrimidin-2-yl) amino) cyclohexane-1-carboxylic acid (294-3). To a solution of tert-butyl-trans-4-((5-fluoro-4-(3-morpholinophenyl) pyrimidin-2-yl) amino) cyclohexane-1-carboxylate (294-2) (100 mg, 219 mol, 1.00 eq) in DCM (3 mL) was added TFA (1 mL) at 25 °C. The resulting mixture was stirred at 25 °C for 0.5 h. LCMS showed that tert-butyl-trans-4-((5-fluoro-4-(3-morpholinophenyl) pyrimidin-2-yl) amino) cyclohexane-1-carboxylate (294-2) was consumed completely and the desired mass was detected. The mixture was concentrated under reduced pressure to give the title compound (294-3) (88.0 mg, crude) as a yellow oil. LCMS (ES⁺): m/z 401.3 [M+H]⁺.

Synthesis of trans-4-((5-fluoro-4-(3-morpholinophenyl) pyrimidin-2-yl) amino) cyclohexane-1-carboxamide (Compound 607). To a solution of trans-4-((5-fluoro-4-(3-morpholinophenyl) pyrimidin-2-yl) amino) cyclohexane-1-carboxylic acid (294-3) (88.0 mg, 220 μmol, 1.00 eq) in DMF (1 mL) were added HATU (41.8 mg, 110 μmol, 0.500 eq) and DIEA (56.8 mg, 440 μmol, 76.6 μL, 2.00 eq) at 25 °C, and then NH₄Cl (11.8 mg, 220 μmol, 1.00 eq) was added to the mixture at 25 °C. The resulting mixture was stirred at 25 °C for 2 h. LCMS showed that trans-4-((5-fluoro-4-(3-morpholinophenyl) pyrimidin-2-yl) amino) cyclohexane-1-carboxylic acid (294-3) was consumed completely and the desired mass was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 30%-60% B over 8.0 min) to give the title compound (Compound 607) (40.1 mg, 95.0 μmol, 43.2% yield, 94.6% purity) as a yellow solid. LCMS (ES⁺): m/z 400.2 [M+H]⁺.

### Example 295. Synthesis of 1-[4-[[5-fluoro-4-(3-morpholinophenyl)pyrimidin-2-yl]amino]-1-piperidyl]ethanone (Compound 606)

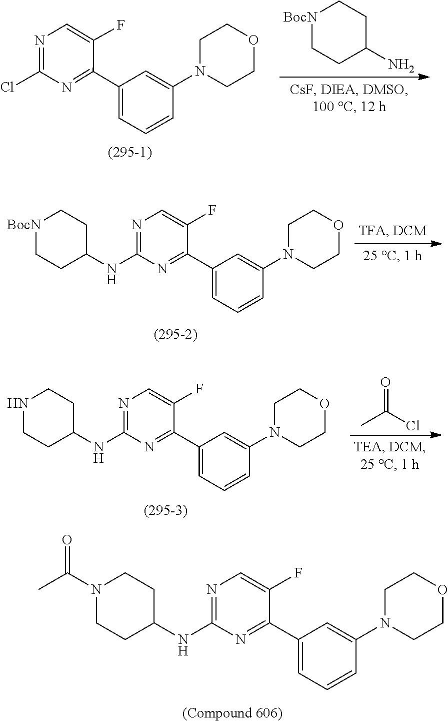

Synthesis of tert-butyl 4-[[5-fluoro-4-(3-morpholinophenyl)pyrimidin-2-yl]amino]piperidine-1-carboxylate (295-2). To a solution of 4-[3-(2-chloro-5-fluoro-pyrimidin-4-yl)phenyl]morpholine (295-1) (500 mg, 1.70 mmol, 1.00 eq) in DMSO (5 mL) was added tert-butyl 4-aminopiperidine-1-carboxylate (409 mg, 2.04 mmol, 1.20 eq) and CsF (517 mg, 3.40 mmol, 2.00 eq) and DIEA (440 mg, 3.40 mmol, 593 μL, 2.00 eq) at 25 °C. The mixture was stirred at 100 °C for 12 h. LC-MS showed the desired mass was detected. The reaction mixture was quenched by ice water 10 mL at 0 °C, and then extracted with EtOAc (10 mL*3). The combined organic layers were washed with brine (5 mL), dried over Na₂SO₄, filtered and concentrated under reduced pressure. The residue was purified by column chromatography (SiO₂, Petroleum ether:Ethyl acetate=1:0 to 3:1) to give the title compound (295-2) (400 mg, 874 μmol, 51.3% yield) as a yellow solid. LCMS (ES⁺): m/z 458.3 [M+H]⁺.

Synthesis of 5-fluoro-4-(3-morpholinophenyl)-N-(4-piperidyl)pyrimidin-2-amine (295-3). To a solution of tert-butyl 4-[[5-fluoro-4-(3-morpholinophenyl)pyrimidin-2-yl]amino]piperidine-1-carboxylate (295-2) (400 mg, 874 μmol, 1.00 eq) in DCM (2 mL) was added TFA (767 mg, 6.73 mmol, 0.50 mL, 7.70 eq). The mixture was stirred at 25 °C for 1 h. LC-MS showed the desired mass was detected. The reaction mixture was concentrated under reduced pressure to give the title compound (295-3) (400 mg, crude, TFA) as a yellow solid. LCMS (ES⁺): m/z 358.1 [M+H]⁺.

Synthesis of 1-[4-[[5-fluoro-4-(3-morpholinophenyl)pyrimidin-2-yl]amino]-1-piperidyl]ethanone (Compound 606). To a solution of 5-fluoro-4-(3-morpholinophenyl)-N-(4-piperidyl)pyrimidin-2-amine (295-3) (200 mg, 424.23 μmol, 1.00 eq, TFA) in DCM (0.5 mL) was added TEA (85.8 mg, 848 μmol, 118 μL, 2.00 eq) and acetyl chloride (33.3 mg, 424 μmol, 30.1 μL, 1.00 eq) at 25 °C. The mixture was stirred at 25 °C for 1 h. LC-MS showed the desired mass was detected. The residue was purified by prep-HPLC (column: Phenomenex luna C18 100*40 mm*3 um; mobile phase: [H₂O (0.2% FA)-ACN]; gradient: 15%-50% B over 8.0 min) to give the title compound (Compound 606) (48.0 mg, 119 μmol, 28.1% yield, 99.4% purity) as a white solid. LCMS (ES⁺): m/z 400.2 [M+H]⁺.

### Example 296. Synthesis of 4-(3-(2-((1-acetylpiperidin-4-yl)amino)-5-fluoropyrimidin-4-yl)phenyl)morpholin-3-one (Compound 206)

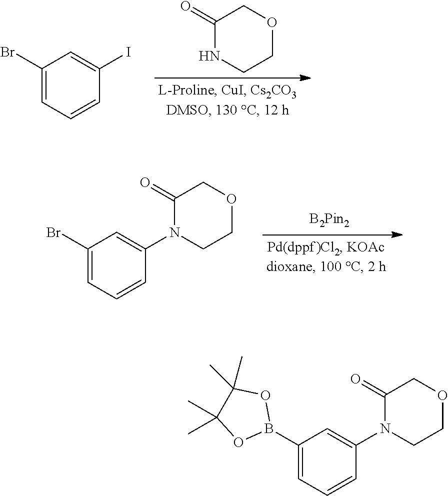 [SMILES: Brc1cccc(I)c1 ; O=C1COCCN1 ; O=C1COCCN1c1cccc(Br)c1 ; CC1(C)OB(c2cccc(N3CCOCC3=O)c2)OC1(C)C]

Synthesis of 4-(3-bromophenyl)morpholin-3-one. A mixture of 1-bromo-3-iodo-benzene (5.00 g, 17.6 mmol, 2.25 mL, 1.00 eq), morpholin-3-one (1.97 g, 19.4 mmol, 1.10 eq), CuI (336 mg, 1.77 mmol, 0.100 eq), L-Proline (201 mg, 1.77 mmol, 0.100 eq) and Cs₂CO₃ (17.2 g, 53.0 mmol, 3.00 eq) in DMSO (100 mL) was degassed and purged with N₂ for 3 times at 25 °C, and then the mixture was stirred at 130 °C for 12 h under N₂ atmosphere. LC-MS showed the desired mass was detected. The mixture was diluted with water (20 mL), and extracted with EtOAc (30 mL*3). The combined organic layers were washed with brine (50 mL), dried over Na₂SO₄, filtered and concentrated under reduced pressure. The residue was purified by column chromatography (SiO₂, Petroleum ether:Ethyl acetate=1:0 to 1:1) to give the title compound (1.10 g, 3.87 mmol, 10.9% yield, 90.0% purity) as a yellow solid. LCMS (ES⁺): m/z 256.0 [M+H]⁺.

Synthesis of 4-(3-(4,4,5,5-tetramethyl-1,3,2-dioxaborolan-2-yl)phenyl)morpholin-3-one. A mixture of 4-(3-bromophenyl)morpholin-3-one (1.08 g, 4.22 mmol, 1.00 eq), 4,4,5,5-tetramethyl-2-(4,4,5,5-tetramethyl-1,3,2-dioxaborolan-2-yl)-1,3,2-dioxaborolane (1.29 g, 5.06 mmol, 1.20 eq), Pd(dppf)Cl₂ (308 mg, 421 μmol, 0.100 eq), KOAc (1.24 g, 12.6 mmol, 3.00 eq) in dioxane (10 mL) was degassed and purged with N₂ for 3 times at 25 °C, and then the mixture was stirred at 100 °C for 2 h under N₂ atmosphere. LC-MS showed the desired mass was detected. The mixture was diluted with water (20 mL), and extracted with EtOAc (30 mL*3). The combined organic layers were washed with brine (50 mL), dried over Na₂SO₄, filtered and concentrated under reduced pressure. The residue was purified by column chromatography (SiO₂, Petroleum ether:Ethyl acetate=1:0 to 1:1) to give the title compound (700 mg, 1.89 mmol, 44.9% yield, 82.0% purity) as a brown solid. LCMS (ES⁺): m/z 304.1 [M+H]⁺.

Synthesis of 4-(3-(2-chloro-5-fluoropyrimidin-4-yl)phenyl)morpholin-3-one. A mixture of 4-[3-(4,4,5,5-tetramethyl-1,3,2-dioxaborolan-2-yl)phenyl]morpholin-3-one (670 mg, 2.21 mmol, 1.00 eq), 2,4-dichloro-5-fluoro-pyrimidine (405 mg, 2.43 mmol, 1.10 eq), Na₂CO₃ (702 mg, 6.63 mmol, 3.00 eq), Pd(dppf)Cl₂ (161 mg, 221 μmol, 0.100 eq) in dioxane (2 mL) and H₂O (1 mL) was degassed and purged with N₂ for 3 times at 25 °C, and then the mixture was stirred at 80 °C for 2 h under N₂ atmosphere. LC-MS showed the desired mass was detected. The mixture was diluted with water (20 mL), and extracted with EtOAc (30 mL*3). The combined organic layers were washed with brine (50 mL), dried over Na₂SO₄, filtered and concentrated under reduced pressure. The residue was purified by column chromatography (SiO₂, Petroleum ether:Ethyl acetate=1:0 to 1:2) to give the title compound (600 mg, 1.48 mmol, 67.0% yield, 76.0% purity) as a brown solid. LCMS (ES⁺): m/z 308.0 [M+H]⁺.

Synthesis of 4-(3-(2-((1-acetylpiperidin-4-yl)amino)-5-fluoropyrimidin-4-yl)phenyl)morpholin-3-one (Compound 206). A mixture of 4-[3-(2-chloro-5-fluoro-pyrimidin-4-yl)phenyl]morpholin-3-one (200 mg, 649 μmol, 1.00 eq), 1-(4-amino-1-piperidyl)ethanone (110 mg, 779 μmol, 1.20 eq), Cs₂CO₃ (635 mg, 1.95 mmol, 3.00 eq), Pd₂(dba)₃ (59.5 mg, 65.0 μmol, 0.100 eq) and BINAP (40.4 mg, 65.0 μmol, 0.100 eq) in dioxane (3 mL) was degassed and purged with N₂ for 3 times at 25 °C, and then the mixture was stirred at 100 °C for 2 h under N₂ atmosphere. LC-MS showed the desired mass was detected. The mixture was diluted with water (10 mL), and extracted with EtOAc (20 mL*3). The combined organic layers were washed with brine (20 mL), dried over Na₂SO₄, filtered and concentrated under reduced pressure. The residue was purified by prep-HPLC (column: Phenomenex Luna C18 100*30 mm*3 um; mobile phase: [H₂O (0.2% FA)-ACN]; gradient: 15%-45% B over 8.0 min) to give the title compound (Compound 206) (68.0 mg, 163 μmol, 25.1% yield, 99.5% purity) as an off-white solid. LCMS (ES⁺): m/z 414.2 [M+H]⁺.

### Example 297. Synthesis of 3-(7-(1-(2-(4-(4-((5-chloro-4-(4′-fluoro-[1,1′-biphenyl]-3-yl)pyrimidin-2-yl)amino)piperidine-1-carbonyl)piperidin-1-yl)acetyl)piperidin-4-yl)-2-oxobenzo[d]oxazol-3 (2H)-yl)piperidine-2,6-dione (Compound 293)

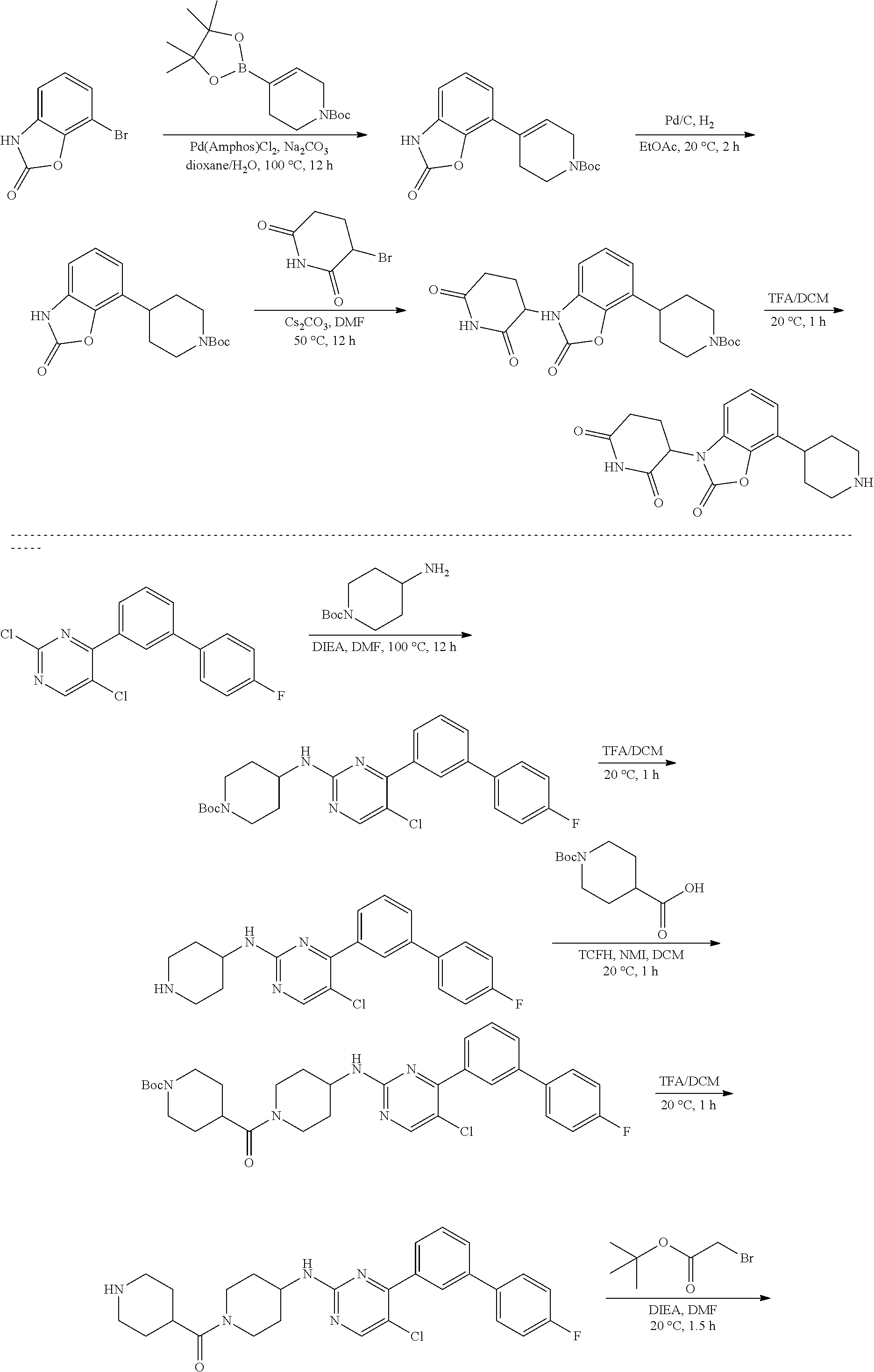

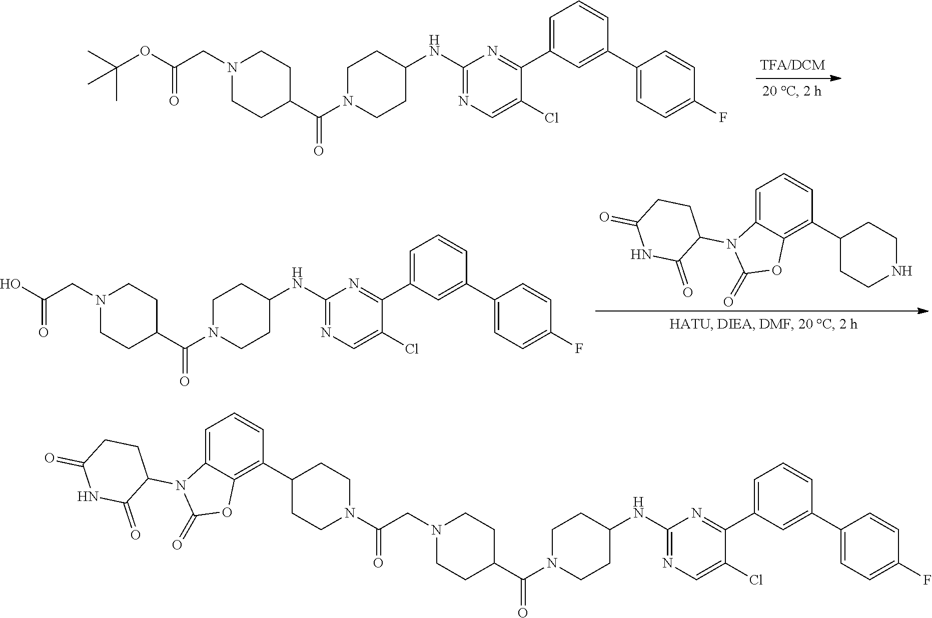 [SMILES: CC(C)(C)OC(=O)CN1CCC(C(=O)N2CCC(Nc3ncc(Cl)c(-c4cccc(-c5ccc(F)cc5)c4)n3)CC2)CC1 ; O=C(O)CN1CCC(C(=O)N2CCC(Nc3ncc(Cl)c(-c4cccc(-c5ccc(F)cc5)c4)n3)CC2)CC1 ; O=C1CCC(n2c(=O)oc3c(C4CCNCC4)cccc32)C(=O)N1 ; O=C1CCC(n2c(=O)oc3c(C4CCN(C(=O)CN5CCC(C(=O)N6CCC(Nc7ncc(Cl)c(-c8cccc(-c9ccc(F)cc9)c8)n7)CC6)CC5)CC4)cccc32)C(=O)N1]

Synthesis of tert-butyl 4-(2-oxo-2,3-dihydrobenzo[d]oxazol-7-yl)-3,6-dihydropyridine-1 (2H)-carboxylate. To a solution of 7-bromobenzo[d]oxazol-2(3H)-one (4.90 g, 22.9 mmol, 1.00 eq) in dioxane (90 mL) and H₂O (30 mL) were added tert-butyl 4-(4,4,5,5-tetramethyl-1,3,2-dioxaborolan-2-yl)-3,6-dihydropyridine-1 (2H)-carboxylate (8.50 g, 27.5 mmol, 1.20 eq), Na₂CO₃ (6.07 g, 57. mmol, 2.50 eq) and Pd(Amphos)Cl₂ (1.62 g, 2.29 mmol, 1.62 mL, 0.100 eq) at 20 °C under N₂ atmosphere. The mixture was stirred at 100 °C for 12 h under N₂. LC-MS showed 7-bromobenzo[d]oxazol-2(3H)-one was consumed completely and the desired mass was detected. The reaction was concentrated under reduced pressure to remove dioxane. Then the reaction mixture was extracted with DCM (30 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=3:1 to 1:1) to give the title compound (6.00 g, 19.0 mmol, 82.8% yield) as a brown solid. LCMS (ES⁺): m/z 217.1 [M+H-100]⁺.

Synthesis of tert-butyl 4-(2-oxo-2,3-dihydrobenzo[d]oxazol-7-yl)piperidine-1-carboxylate. 10% Pd/C (10.5 g, 50% purity) was added to EtOAc (20 mL) at 20 °C under N₂ atmosphere, then tert-butyl 4-(2-oxo-2,3-dihydrobenzo[d]oxazol-7-yl)-3,6-dihydropyridine-1 (2H)-carboxylate (3.50 g, 11.1 mmol, 1.00 eq) was added to the mixture at 20 °C, the suspension was degassed and purged with H₂ for three times. The mixture was stirred at 20 °C for 2 h under H₂ (15 psi) atmosphere. LC-MS showed tert-butyl 4-(2-oxo-2,3-dihydrobenzo[d]oxazol-7-yl)-3,6-dihydropyridine-1 (2H)-carboxylate was consumed completely and the desired mass was detected. The reaction mixture was filtered and the filtrate was concentrated under reduced pressure to give the title compound (300 mg, crude, TFA) as a brown solid. LCMS (ES⁺): m/z 263.1 [M+H−56]⁺.

Synthesis of tert-butyl 4-(3-(2,6-dioxopiperidin-3-yl)-2-oxo-2,3-dihydrobenzo[d]oxazol-7-yl)piperidine-1-carboxylate. To a solution of tert-butyl 4-(2-oxo-2,3-dihydrobenzo[d]oxazol-7-yl)piperidine-1-carboxylate (1.00 g, 3.14 mmol, 1.00 eq) in DMF (5 mL) were added Cs₂CO₃ (2.05 g, 6.28 mmol, 2.00 eq) and 3-bromopiperidine-2,6-dione (905 mg, 4.71 mmol, 1.50 eq) at 20 °C. The mixture was stirred at 50 °C for 12 h. LC-MS showed tert-butyl 4-(2-oxo-2,3-dihydrobenzo[d]oxazol-7-yl)piperidine-1-carboxylate was consumed completely and the desired mass was detected. Water (30 mL) was added to the mixture, filtered and the filter cake was dried under reduced pressure to give the title compound (850 mg, crude) as a blue solid. LCMS (ES⁺): m/z 374.1 [M+H−56]⁺.

Synthesis of 3-(2-oxo-7-(piperidin-4-yl)benzo[d]oxazol-3 (2H)-yl)piperidine-2,6-dione. To a solution of tert-butyl 4-(3-(2,6-dioxopiperidin-3-yl)-2-oxo-2,3-dihydrobenzo[d]oxazol-7-yl)piperidine-1-carboxylate (350 mg, 815 μmol, 1.00 eq) in DCM (3 mL) was added TFA (3.58 g, 31.4 mmol, 2.33 mL, 38.5 eq) at 20 °C. The mixture was stirred at 20 °C for 1 h. LC-MS showed tert-butyl 4-(3-(2,6-dioxopiperidin-3-yl)-2-oxo-2,3-dihydrobenzo[d]oxazol-7-yl)piperidine-1-carboxylate was consumed completely and the desired mass was detected. The reaction mixture was concentrated under reduced pressure to remove solvent to give the title compound (361 mg, crude, TFA) as a brown oil. LCMS (ES⁺): m/z 330.2 [M+H]⁺.

Synthesis of tert-butyl 4-((5-chloro-4-(4′-fluoro-[1,1′-biphenyl]-3-yl)pyrimidin-2-yl)amino)piperidine-1-carboxylate. To a solution of tert-butyl 4-aminopiperidine-1-carboxylate (2.51 g, 12.5 mmol, 1.00 eq) in DMF (80 mL) were added DIEA (4.86 g, 37.6 mmol, 6.55 mL, 3.00 eq) and 2,5-dichloro-4-(4′-fluoro-[1,1′-biphenyl]-3-yl)pyrimidine (4.00 g, 12.5 mmol, 1.00 eq) at 20 °C. The mixture was stirred at 100 °C for 12 h. LCMS showed that 2,5-dichloro-4-(4′-fluoro-[1,1′-biphenyl]-3-yl)pyrimidine was consumed and the desired mass was detected. To the mixture was added H₂O (100 mL) and the mixture was extracted with EtOAc (100 mL*3). The combined organic layers were washed with brine (200 mL*3), dried over Na₂SO₄, filtered and the filtrate was concentrated in vacuo to give the crude product. The crude product was purified by MPLC (SiO₂, Petroleum ether:Ethyl acetate=1:0 to 0:1) to give the title compound (4.80 g, 9.94 mmol, 79.3% yield) as a yellow oil. LCMS (ES⁺): m/z 483.2 [M+H]⁺

Synthesis of 5-chloro-4-(4′-fluoro-[1,1′-biphenyl]-3-yl)-N-(piperidin-4-yl)pyrimidin-2-amine. To a solution of tert-butyl 4-((5-chloro-4-(4′-fluoro-[1,1′-biphenyl]-3-yl)pyrimidin-2-yl)amino)piperidine-1-carboxylate (500 mg, 1.04 mmol, 1.00 eq) in DCM (9 mL) was added TFA (4.61 g, 40.4 mmol, 3 mL, 39.0 eq) at 20 °C. The mixture was stirred at 20 °C for 1 h. LC-MS showed tert-butyl 4-((5-chloro-4-(4′-fluoro-[1,1′-biphenyl]-3-yl)pyrimidin-2-yl)amino)piperidine-1-carboxylate was consumed completely and the desired mass was detected. The reaction mixture was concentrated under reduced pressure to give the title compound (514 mg, crude, TFA) as a yellow oil. LCMS (ES⁺): m/z 383.2 [M+H]⁺.

Synthesis of tert-butyl 4-(4-((5-chloro-4-(4′-fluoro-[1,1′-biphenyl]-3-yl)pyrimidin-2-yl)amino)piperidine-1-carbonyl)piperidine-1-carboxylate. To a solution of 5-chloro-4-(4′-fluoro-[1,1′-biphenyl]-3-yl)-N-(piperidin-4-yl)pyrimidin-2-amine (514 mg, 1.03 mmol, 1.00 eq, TFA) in DCM (10 mL) were added 1-(tert-butoxycarbonyl)piperidine-4-carboxylic acid (237 mg, 1.03 mmol, 1.00 eq), TCFH (436 mg, 1.55 mmol, 1.50 eq) and NMI (255 mg, 3.10 mmol, 247 μL, 3.00 eq) at 20 °C. The mixture was stirred at 20 °C for 1 h. LC-MS showed 5-chloro-4-(4′-fluoro-[1,1′-biphenyl]-3-yl)-N-(piperidin-4-yl)pyrimidin-2-amine was consumed completely and the desired mass was detected. Water (10 mL) was added to the mixture at 20 °C. Then the reaction mixture was extracted with DCM (5 mL*3). The organic layers were dried over anhydrous sodium sulfate, filtered and the filtrate was concentrated under reduced pressure to give a residue. The residue was purified by prep-TLC (SiO₂, Ethyl acetate:Petroleum ether=2:1) to give the title compound (550 mg, 926 μmol, 89.5% yield) as a colorless oil. LCMS (ES⁺): m/z 594.4 [M+H]⁺

Synthesis of (4-((5-chloro-4-(4′-fluoro-[1,1′-biphenyl]-3-yl)pyrimidin-2-yl)amino)piperidin-1-yl)(piperidin-4-yl)methanone. To a solution of 4-(4-((5-chloro-4-(4′-fluoro-[1,1′-biphenyl]-3-yl)pyrimidin-2-yl)amino)piperidine-1-carbonyl)piperidine-1-carboxylate (200 mg, 337 μmol, 1.00 eq) in DCM (3 mL) was added TFA (3.07 g, 26.9 mmol, 2.00 mL, 80.0 eq) at 20 °C. The mixture was stirred at 20 °C for 1 h. LC-MS showed 4-(4-((5-chloro-4-(4′-fluoro-[1,1′-biphenyl]-3-yl)pyrimidin-2-yl)amino)piperidine-1-carbonyl)piperidine-1-carboxylate was consumed completely and the desired mass was detected. The reaction mixture was concentrated under reduced pressure to remove solvent to give the title compound (204 mg, crude, TFA) as a yellow oil. LCMS (ES⁺): m/z 494.3 [M+H]⁺.

Synthesis of tert-butyl 2-(4-(4-((5-chloro-4-(4′-fluoro-[1,1′-biphenyl]-3-yl)pyrimidin-2-yl)amino)piperidine-1-carbonyl)piperidin-1-yl)acetate. To a solution of (4-((5-chloro-4-(4′-fluoro-[1,1′-biphenyl]-3-yl)pyrimidin-2-yl)amino)piperidin-1-yl)(piperidin-4-yl)methanone (204 mg, 336 μmol, 1.00 eq, TFA) in DMF (5 mL) were added tert-butyl 2-bromoacetate (78.5 mg, 403 μmol, 59.5 μL, 1.20 eq) and DIEA (173 mg, 1.34 mmol, 234 μL, 4.00 eq) at 20 °C. The mixture was stirred at 20 °C for 1.5 h. LC-MS showed (4-((5-chloro-4-(4′-fluoro-[1,1′-biphenyl]-3-yl)pyrimidin-2-yl)amino)piperidin-1-yl)(piperidin-4-yl)methanone was consumed completely and the desired mass was detected. Water (5 mL) was added to the mixture at 20 °C. Then the reaction mixture was extracted with DCM (3 mL*3). The organic layers were dried over anhydrous sodium sulfate, filtered and the filtrate was concentrated under reduced pressure to give a residue. The residue was purified by prep-TLC (SiO₂, DCM:MeOH=10:1) to give the title compound (150 mg, 247 μmol, 73.5% yield) as a white solid. LCMS (ES⁺): m/z 608.4 [M+H]⁺.

Synthesis of 2-(4-(4-((5-chloro-4-(4′-fluoro-[1,1′-biphenyl]-3-yl)pyrimidin-2-yl)amino)piperidine-1-carbonyl)piperidin-1-yl)acetic acid. To a solution of tert-butyl 2-(4-(4-((5-chloro-4-(4′-fluoro-[1,1′-biphenyl]-3-yl)pyrimidin-2-yl)amino)piperidine-1-carbonyl)piperidin-1-yl)acetate (150 mg, 247 μmol, 1.00 eq) in DCM (3 mL) was added TFA (1.54 g, 13.5 mmol, 1 mL, 54.6 eq) at 20 °C. The mixture was stirred at 20 °C for 2 h. LC-MS showed tert-butyl 2-(4-(4-((5-chloro-4-(4′-fluoro-[1,1′-biphenyl]-3-yl)pyrimidin-2-yl)amino)piperidine-1-carbonyl)piperidin-1-yl)acetate was consumed completely and the desired mass was detected. The reaction mixture was concentrated under reduced pressure to give the title compound (136 mg, crude) as a yellow oil. LCMS (ES⁺): m/z 552.3 [M+H]⁺.

Synthesis of 3-(7-(1-(2-(4-(4-((5-chloro-4-(4′-fluoro-[1,1′-biphenyl]-3-yl)pyrimidin-2-yl)amino)piperidine-1-carbonyl)piperidin-1-yl)acetyl)piperidin-4-yl)-2-oxobenzo[d]oxazol-3 (2H)-yl)piperidine-2,6-dione (Compound 293). To a solution of 2-(4-(4-((5-chloro-4-(4′-fluoro-[1,1′-biphenyl]-3-yl)pyrimidin-2-yl)amino)piperidine-1-carbonyl)piperidin-1-yl)acetic acid (136 mg, 246 μmol, 1.00 eq) in DMF (0.5 mL) were added HATU (141 mg, 370 μmol, 1.50 eq), DIEA (95.5 mg, 739 μmol, 129 μL, 3.00 eq) and 3-[2-oxo-7-(4-piperidyl)-1,3-benzoxazol-3-yl]piperidine-2,6-dione (109 mg, 246 μmol, 1.00 eq, TFA) at 20 °C. The mixture was stirred at 20 °C for 2 h. LC-MS showed 2-(4-(4-((5-chloro-4-(4′-fluoro-[1,1′-biphenyl]-3-yl)pyrimidin-2-yl)amino)piperidine-1-carbonyl)piperidin-1-yl)acetic acid was consumed completely and the desired mass was detected. The reaction mixture was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 40%-70% B over 8.0 min) to give the title compound (Compound 293) (26.9 mg, 29.9 μmol, 12.1% yield, 96.0% purity) as a white solid. LCMS (ES⁺): m/z 863.3 [M+H]⁺

### Example 298. Synthesis of 3-(4-(4-(2-(4-(4-((5-chloro-4-(4′-fluoro-[1,1′-biphenyl]-3-yl)pyrimidin-2-yl)amino)piperidine-1-carbonyl)piperidin-1-yl)-2-oxoethyl)-4-hydroxypiperidin-1-yl)-3-fluorophenyl)piperidine-2,6-dione (Compound 282)

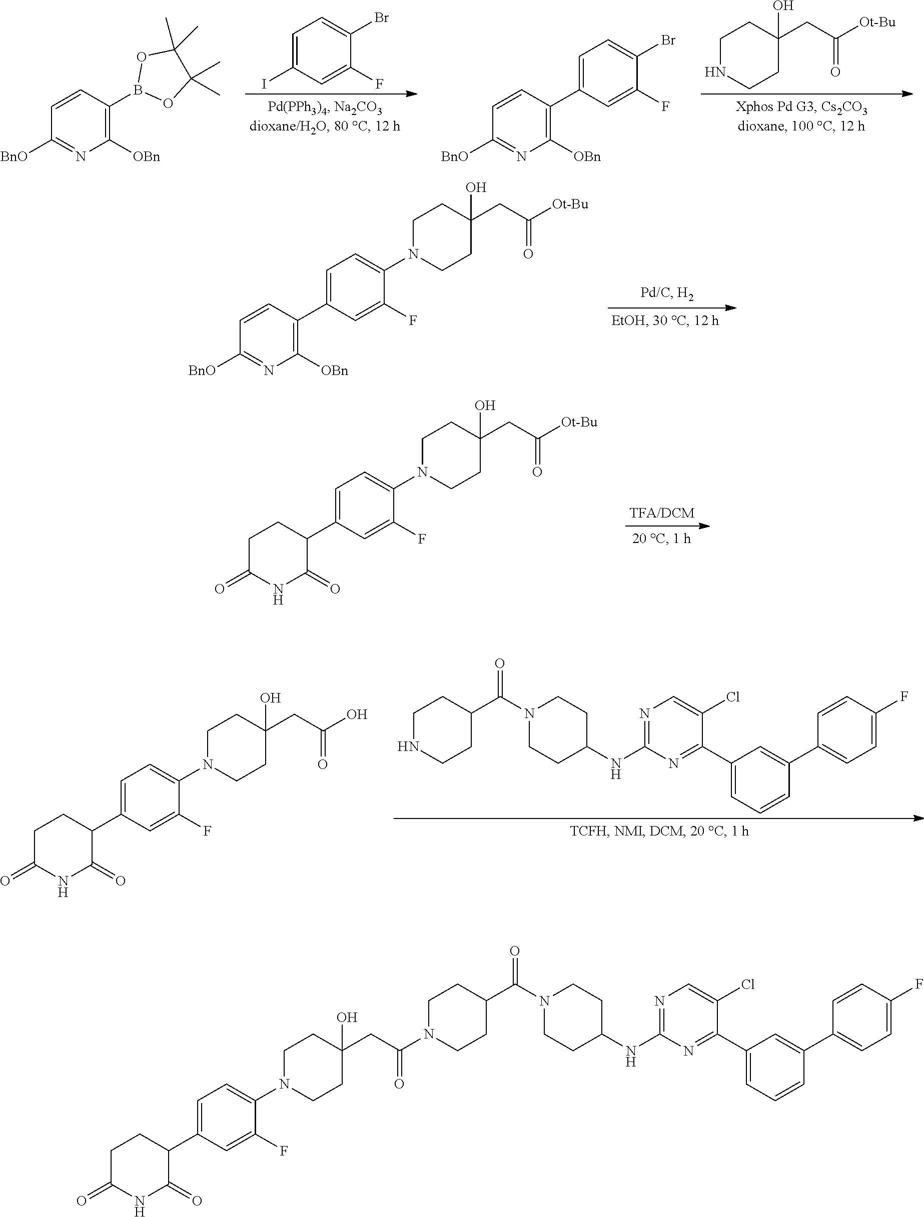

Synthesis of 2,6-bis(benzyloxy)-3-(4-bromo-3-fluorophenyl)pyridine. To a solution of 1-bromo-2-fluoro-4-iodobenzene (20 g, 66.5 mmol, 1.00 eq) and 2,6-bis(benzyloxy)-3-(4,4,5,5-tetramethyl-1,3,2-dioxaborolan-2-yl)pyridine (27.7 g, 66.5 mmol, 1.00 eq) in dioxane (500 mL) and H₂O (50 mL) were added Na₂CO₃ (21.1 g, 199 mmol, 3.00 eq) and Pd(PPh₃)₄ (3.84 g, 3.32 mmol, 0.0500 eq) at 20 °C under N₂ atmosphere, the mixture was stirred at 80 °C for 12 h under N₂. LCMS showed that the desired mass was detected. The reaction mixture was concentrated under reduced pressure to remove solvent. Water (500 mL) was added to the mixture at 25 °C. Then the reaction mixture was extracted with EtOAc (500 mL*3). The organic layers were dried over anhydrous sodium sulfate, filtered and the filtrate was concentrated under reduced pressure to give a residue. The residue was purified by MPLC (SiO₂, Petroleum ether:Ethyl acetate=1:0 to 1:1) to give the title compound (15 g, 32.3 mmol, 48.6% yield) as a yellow oil. LCMS (ES⁺): m/z 464.1.

Synthesis of tert-butyl 2-(1-(4-(2,6-bis(benzyloxy)pyridin-3-yl)-2-fluorophenyl)-4-hydroxypiperidin-4-yl)acetate. To a solution of 2,6-bis(benzyloxy)-3-(4-bromo-3-fluorophenyl)pyridine (2.00 g, 2.15 mmol, 1.00 eq) in dioxane (50 mL) were added tert-butyl 2-(4-hydroxypiperidin-4-yl)acetate (1.39 g, 6.46 mmol, 3.00 eq), XPhos Pd G3 (182 mg, 215 μmol, 0.100 eq) and Cs₂CO₃ (2.11 g, 6.46 mmol, 3.00 eq) at 20 °C under N₂ atmosphere. The mixture was stirred at 100 °C for 12 h under N₂. LC-MS showed 2,6-bis(benzyloxy)-3-(4-bromo-3-fluorophenyl)pyridine was consumed completely and the desired mass was detected. The reaction mixture was concentrated under reduced pressure to remove solvent. Water (30 mL) was added to the mixture at 20 °C. Then the reaction mixture was extracted with EtOAc (20 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=10:1 to 5:1) to give the title compound (890 mg, 1.49 mmol, 69.0% yield) as a yellow oil. LCMS (ES⁺): m/z 599.3 [M+H]⁺.

Synthesis of tert-butyl 2-(1-(4-(2,6-dioxopiperidin-3-yl)-2-fluorophenyl)-4-hydroxypiperidin-4-yl)acetate. 10% Pd/C (2.00 g, 50% purity) was added to EtOH (10 mL) and at 20 °C under N₂ atmosphere, then tert-butyl 2-(1-(4-(2,6-bis(benzyloxy)pyridin-3-yl)-2-fluorophenyl)-4-hydroxypiperidin-4-yl)acetate (890 mg, 1.49 mmol, 1.00 eq) was added to the mixture at 20 °C, the suspension was degassed and purged with H₂ for three times. The mixture was stirred at 30 °C for 12 h under H₂ (15 psi) atmosphere. LC-MS showed tert-butyl 2-(1-(4-(2,6-bis(benzyloxy)pyridin-3-yl)-2-fluorophenyl)-4-hydroxypiperidin-4-yl)acetate was consumed completely and the desired mass was detected. The reaction mixture was filtered and the filtrate was concentrated under reduced pressure to give the title compound (640 mg, crude) as a white solid. LCMS (ES⁺): m/z 421.4 [M+H]⁺.

Synthesis of 2-(1-(4-(2,6-dioxopiperidin-3-yl)-2-fluorophenyl)-4-hydroxypiperidin-4-yl)acetic acid. To a solution of tert-butyl 2-(1-(4-(2,6-dioxopiperidin-3-yl)-2-fluorophenyl)-4-hydroxypiperidin-4-yl)acetate (440 mg, 1.05 mmol, 1.00 eq) in DCM (9 mL) was added TFA (9.21 g, 80.8 mmol, 6.00 mL, 77.2 eq) at 20 °C. The mixture was stirred at 20 °C for 1 h. LC-MS showed tert-butyl 2-(1-(4-(2,6-dioxopiperidin-3-yl)-2-fluorophenyl)-4-hydroxypiperidin-4-yl)acetate was consumed completely and the desired mass was detected. The reaction mixture was concentrated under reduced pressure to remove solvent to give the title compound (381 mg, crude) as a yellow oil. LCMS (ES⁺): m/z 365.1 [M+H]⁺.

Synthesis of 3-(4-(4-(2-(4-(4-((5-chloro-4-(4′-fluoro-[1,1′-biphenyl]-3-yl)pyrimidin-2-yl)amino)piperidine-1-carbonyl)piperidin-1-yl)-2-oxoethyl)-4-hydroxypiperidin-1-yl)-3-fluorophenyl)piperidine-2,6-dione (Compound 282). To a solution of (4-((5-chloro-4-(4′-fluoro-[1,1′-biphenyl]-3-yl)pyrimidin-2-yl)amino)piperidin-1-yl)(piperidin-4-yl)methanone (279 mg, 459 μmol, 0.880 eq, TFA) in DCM (7 mL) were added 2-(1-(4-(2,6-dioxopiperidin-3-yl)-2-fluorophenyl)-4-hydroxypiperidin-4-yl)acetic acid (190 mg, 521 μmol, 1.00 eq), TCFH (219 mg, 782 μmol, 1.50 eq) and NMI (128 mg, 1.56 mmol, 125 μL, 3.00 eq) at 20 °C. The mixture was stirred at 20 °C for 1 h. LC-MS showed (4-((5-chloro-4-(4′-fluoro-[1,1′-biphenyl]-3-yl)pyrimidin-2-yl)amino)piperidin-1-yl)(piperidin-4-yl)methanone was consumed completely and the desired mass was detected. The reaction mixture was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 40%-70% B over 8.0 min) to give the title compound (Compound 282) (104 mg, 122 μmol, 23.4% yield, 98.9% purity) as a white solid. LCMS (ES⁺): m/z 840.3 [M+H]⁺.

### Example 299. Synthesis of 3-(6-(1-(2-(4-(4-((5-chloro-4-(4′-fluoro-[1,1′-biphenyl]-3-yl)pyrimidin-2-yl)amino)piperidine-1-carbonyl)-4-hydroxypiperidin-1-yl)acetyl)piperidin-4-yl)-1-methyl-1H-indazol-3-yl)piperidine-2,6-dione (Compound 289)

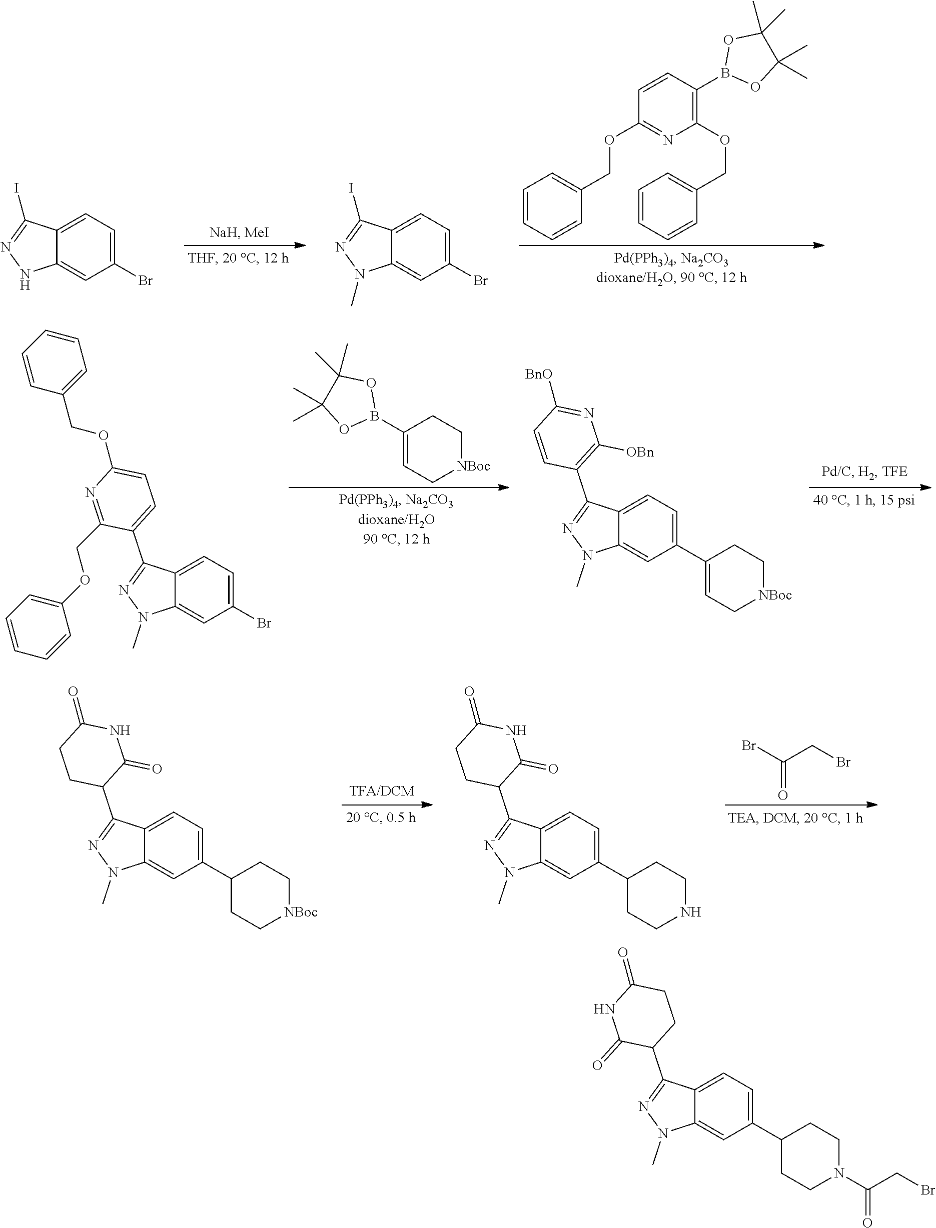

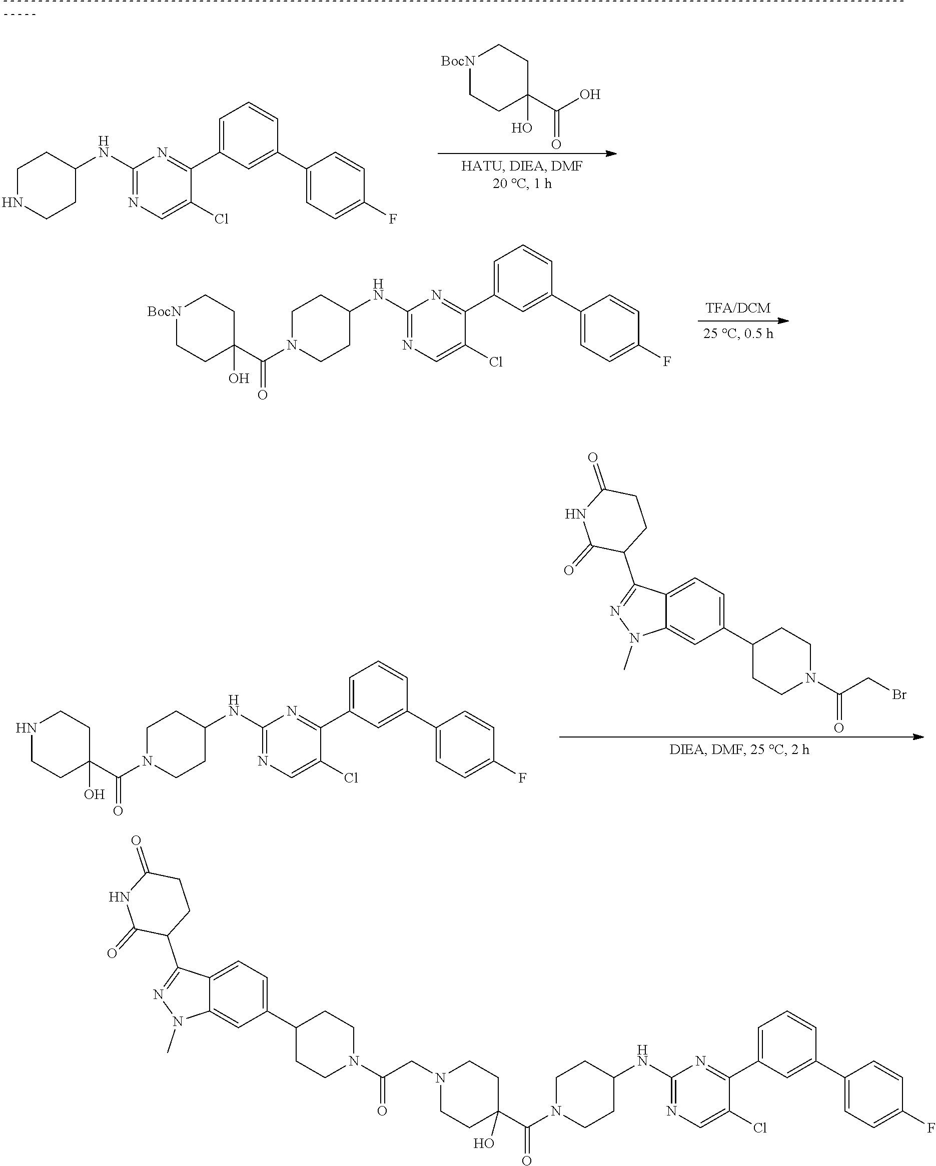

Synthesis of 6-bromo-3-iodo-1-methyl-1H-indazole. This reaction was carried out in 2 batches in parallel. To a solution of 6-bromo-3-iodo-1H-indazole (80 g, 248 mmol, 1.00 eq) in THF (500 mL) was added NaH (11.9 g, 297 mmol, 60% purity, 1.20 eq) at 25 °C for 0.5 h, and then CH₃I (70.3 g, 495 mmol, 30.8 mL, 2.00 eq) was added dropwise at 25 °C under N₂. The mixture was stirred at 25 °C for 1.5 hours under N₂. LCMS indicated 6-bromo-3-iodo-1H-indazole was consumed completely and desired product was detected. The two combined reaction was quenched with saturated NH₄Cl (1.0 L) and the aqueous phase was extracted with ethyl acetate (500 mL×3). The combined organic phase washed with brine (500 mL×2), dried over Na₂SO₄, filtered. The filtrate was concentrated to get a residue. The residue was purified by column chromatography (SiO₂, Petroleum ether/Ethyl acetate=0/1 to 5/1) to give (130 g, 386 mmol, 77.9% yield) as a white solid. LCMS (ES⁺): m/z 336.8 [M+H]⁺.

Synthesis of 3-(2,6-bis(benzyloxy)pyridin-3-yl)-6-bromo-1-methyl-1H-indazole. A mixture of 6-bromo-3-iodo-1-methyl-1H-indazole (130 g, 386 mmol, 1.00 eq), 2,6-dibenzyloxy-3-(4,4,5,5-tetramethyl-1,3,2-dioxaborolan-2-yl)pyridine (144.9 g, 347 mmol, 0.90 eq), Na₂CO₃ (123 g, 1.16 mol, 3.00 eq) and palladium; triphenylphosphane (22.3 g, 19.3 mmol, 0.05 eq) in 1,4-dioxane (400 mL) and H₂O (100 mL) was degassed and purged with N₂ for 3 times, and then the mixture was stirred at 90 °C for 12 hours under N₂ atmosphere. LCMS indicated 6-bromo-3-iodo-1-methyl-1H-indazole was consumed completely desired product was detected. The mixture was poured into saturated aq. NH₄Cl (200 mL) with stirring and the aqueous phase was extracted with ethyl acetate (100 mL×3). The combined organic phases were washed with brine (100 mL×2), dried over Na₂SO₄, filtered and concentrated under reduced pressure to give a residue. The residue was purified by column chromatography (SiO₂, Petroleum ether/Ethyl acetate=0/1 to 5/1) to give (80 g, 157 mmol, 40.8% yield, 98.5% purity) as a white solid. LCMS (ES⁺): m/z 500.1 [M+H]⁺.

Synthesis of tert-butyl 4-(3-(2,6-bis(benzyloxy)pyridin-3-yl)-1-methyl-1H-indazol-6-yl)-3,6-dihydropyridine-1(2H)-carboxylate. To a solution of 3-(2,6-bis(benzyloxy)pyridin-3-yl)-6-bromo-1-methyl-1H-indazole (10.0 g, 20.0 mmol, 1.00 eq) and tert-butyl 4-(4,4,5,5-tetramethyl-1,3,2-dioxaborolan-2-yl)-3,6-dihydropyridine-1(2H)-carboxylate (6.18 g, 20.0 mmol, 1.00 eq) in dioxane (100 mL) and H₂O (20 mL) were added Pd(PPh₃)₄ (2.31 g, 2.00 mmol, 0.100 eq) and Na₂CO₃ (4.24 g, 40.0 mmol, 2.00 eq) at 20 °C under N₂, the mixture was stirred at 90 °C for 12 h under N₂. LCMS showed 3-(2,6-bis(benzyloxy)pyridin-3-yl)-6-bromo-1-methyl-1H-indazole was consumed and the desired target MS was detected. H₂O (60 mL) was added to the solution, the mixture was extracted with EtOAc (60 mL*3), then the combined organic layers were dried over Na₂SO₄, filtered and the filtrate was concentrated under reduced pressure to give a residue. The residue was purified by MPLC (SiO₂, Petroleum ether/Ethyl acetate=1/0 to 0/1) to give the title compound (7.60 g, 12.6 mmol, 63.1% yield) as a yellow solid. LCMS (ES⁺): m/z 603.4 [M+H]⁺

Synthesis of tert-butyl 4-(3-(2,6-dioxopiperidin-3-yl)-1-methyl-1H-indazol-6-yl)piperidine-1-carboxylate. To a solution tert-butyl 4-(3-(2,6-bis(benzyloxy)pyridin-3-yl)-1-methyl-1H-indazol-6-yl)-3,6-dihydropyridine-1(2H)-carboxylate (3.60 g, 5.97 mmol, 1.00 eq) in TFE (80 mL) was added 10% Pd/C (6.00 g, 50% purity) at 20 °C. The suspension was degassed and purged with H₂ for 3 times. The mixture was stirred under H₂ (15 psi) at 40 °C for 1 h. LCMS showed tert-butyl 4-(3-(2,6-bis(benzyloxy)pyridin-3-yl)-1-methyl-1H-indazol-6-yl)-3,6-dihydropyridine-1(2H)-carboxylate was consumed and the desired target MS was detected. The mixture was filtered, the filtrate was concentrated under reduced pressure to give the title compound (3.00 g, crude) as a yellow oil. LCMS (ES⁺): m/z 427.2 [M+H]⁺

Synthesis of 3-(1-methyl-6-(piperidin-4-yl)-1H-indazol-3-yl)piperidine-2,6-dione. A solution of tert-butyl 4-(3-(2,6-dioxopiperidin-3-yl)-1-methyl-1H-indazol-6-yl)piperidine-1-carboxylate (3.00 g, 7.03 mmol, 1.00 eq) in TFA (10 mL) and DCM (30 mL) was stirred at 20 °C for 0.5 h. LCMS showed tert-butyl 4-(3-(2,6-dioxopiperidin-3-yl)-1-methyl-1H-indazol-6-yl)piperidine-1-carboxylate was consumed and the desired target MS was detected. The mixture was concentrated under reduced pressure to give the title compound (3.00 g, crude, TFA) as a yellow oil. LCMS (ES⁺): m/z 327.1 [M+H]⁺

Synthesis of 3-(6-(1-(2-bromoacetyl)piperidin-4-yl)-1-methyl-1H-indazol-3-yl)piperidine-2,6-dione. To a solution of 3-(1-methyl-6-(piperidin-4-yl)-1H-indazol-3-yl)piperidine-2,6-dione (300 mg, 919 μmol, 1.00 eq) in DCM (5.00 mL) was added TEA (186 mg, 1.84 mmol, 255 μL, 2.00 eq) and 2-bromoacetyl bromide (185 mg, 919 μmol, 80.0 μL, 1.00 eq) at 20 °C. The mixture was stirred at 20 °C for 1 h. LC-MS showed the desired mass was detected. The reaction mixture was filtered and the filtrate concentrated under high vacuum to give the title compound (250 mg, 558 μmol, 60.8% yield) as a yellow oil. LCMS (ES⁺): m/z 447.1, 449.1]⁺.

Synthesis of tert-butyl 4-(4-((5-chloro-4-(4′-fluoro-[1,1′-biphenyl]-3-yl)pyrimidin-2-yl)amino)piperidine-1-carbonyl)-4-hydroxypiperidine-1-carboxylate. A mixture of 5-chloro-4-(4′-fluoro-[1,1′-biphenyl]-3-yl)-N-(piperidin-4-yl)pyrimidin-2-amine (1.00 g, 2.61 mmol, 1.00 eq), 1-tert-butoxycarbonyl-4-hydroxy-piperidine-4-carboxylic acid (1.28 g, 5.22 mmol, 2.00 eq), HATU (993 mg, 2.61 mmol, 1.00 eq), DIEA (675 mg, 5.22 mmol, 909 μL, 2.00 eq) in DMF (10 mL), and then the mixture was stirred at 20 °C for 1 h under N₂ atmosphere. LC-MS showed the desired mass was detected. The reaction mixture was quenched by addition H₂O 20 mL, and then extracted with DCM (20 mL*3). The combined organic layers were dried over Na₂SO₄, filtered and concentrated under reduced pressure. The residue was purified by column chromatography to give the title compound (700 mg, 1.15 mmol, 43.9% yield) as a white solid. LCMS (ES⁺): m/z 610.3[M+H]⁺.

Synthesis of (4-((5-chloro-4-(4′-fluoro-[1,1′-biphenyl]-3-yl)pyrimidin-2-yl)amino)piperidin-1-yl)(4-hydroxypiperidin-4-yl)methanone. To a solution of tert-butyl 4-(4-((5-chloro-4-(4′-fluoro-[1,1′-biphenyl]-3-yl)pyrimidin-2-yl)amino)piperidine-1-carbonyl)-4-hydroxypiperidine-1-carboxylate (700 mg, 1.15 mmol, 1.00 eq) in DCM (2 mL) was added TFA (2.15 g, 18.8 mmol, 1.40 mL, 16.4 eq). The mixture was stirred at 25 °C for 0.5 h. LC-MS showed the desired mass was detected. The reaction mixture was concentrated under reduced pressure to give the title compound (1.20 g, crude) as a yellow oil. LCMS (ES⁺): m/z 510.2 [M+H]⁺.

Synthesis of 3-(6-(1-(2-(4-(4-((5-chloro-4-(4′-fluoro-[1,1′-biphenyl]-3-yl)pyrimidin-2-yl)amino)piperidine-1-carbonyl)-4-hydroxypiperidin-1-yl)acetyl)piperidin-4-yl)-1-methyl-1H-indazol-3-yl)piperidine-2,6-dione (Compound 289). To a solution of (4-((5-chloro-4-(4′-fluoro-[1,1′-biphenyl]-3-yl)pyrimidin-2-yl)amino)piperidin-1-yl)(4-hydroxypiperidin-4-yl)methanone (200 mg, 392 μmol, 1.00 eq), 3-[6-[1-(2-bromoacetyl)-4-piperidyl]-1-methyl-indazol-3-yl]piperidine-2,6-dione (192 mg, 431 μmol, 1.10 eq) in DMF (1 mL) was added DIEA (50.6 mg, 392 μmol, 68.3 μL, 1.00 eq) at 25 °C. The mixture was stirred at 25 °C for 2 h. LC-MS showed the desired mass was detected. The reaction mixture was quenched by addition H₂O 5 mL, and then diluted with H₂O 5 mL and extracted with EtOAc (5 mL*3). The combined organic layers were dried over Na₂SO₄, filtered and concentrated under reduced pressure. The residue was purified by prep-HPLC (column: Phenomenex luna C18 100*40 mm*3 um; mobile phase: [H₂O (0.1% TFA)-ACN]; gradient: 20%-50% B over 8.0 min] to give the title compound (Compound 289) (20.3 mg, 20.3 μmol, 5.18% yield, 99.18% purity, TFA) as a white solid. LCMS (ES⁺): m/z 876.1 [M+H]⁺.

### Example 300. Synthesis of 3-(6-(1-(2-(4-(4-((5-chloro-4-(4′-fluoro-[1,1′-biphenyl]-3-yl)pyrimidin-2-yl)amino)piperidine-1-carbonyl)-4-hydroxypiperidin-1-yl)-2-oxoethyl)piperidin-4-yl)-1-methyl-1H-indazol-3-yl)piperidine-2,6-dione (Compound 290)

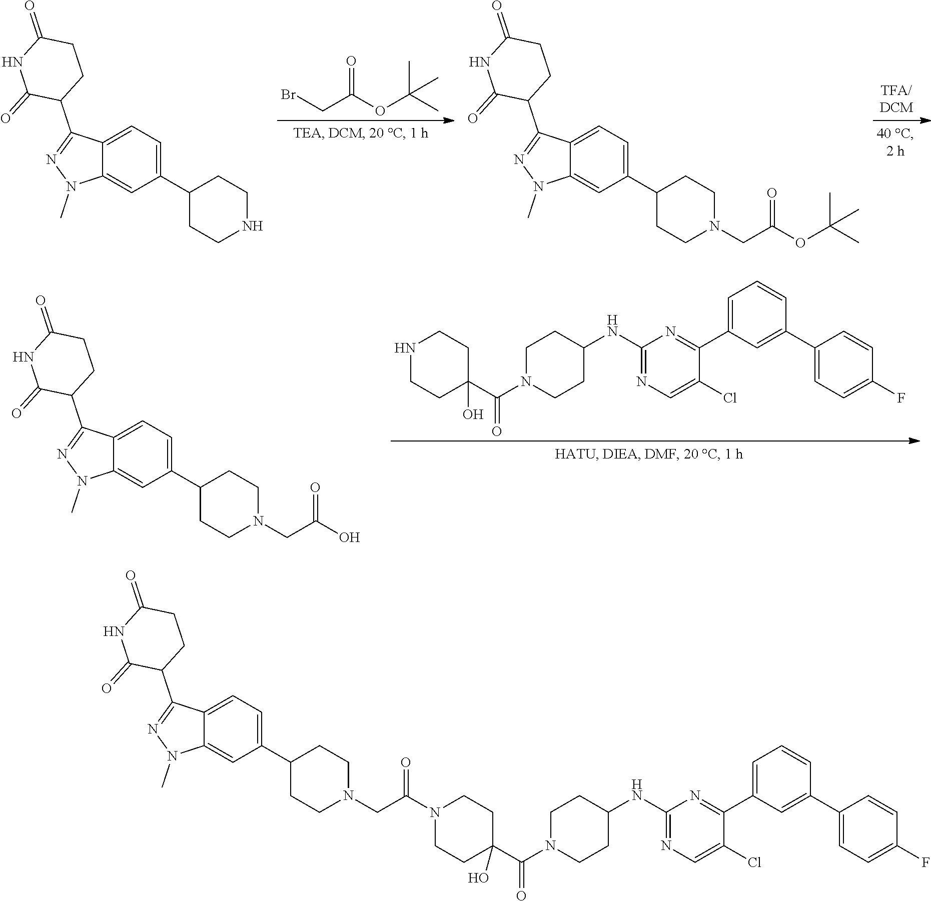

Synthesis of tert-butyl 2-(4-(3-(2,6-dioxopiperidin-3-yl)-1-methyl-1H-indazol-6-yl)piperidin-1-yl)acetate. To a solution of 3-(1-methyl-6-(piperidin-4-yl)-1H-indazol-3-yl)piperidine-2,6-dione (500 mg, 1.53 mmol, 1.00 eq) in DCM (5 mL) was added TEA (310 mg, 3.06 mmol, 426 μL, 2.00 eq) and tert-butyl 2-bromoacetate (298 mg, 1.53 mmol, 226 μL, 1.00 eq) at 20 °C. The mixture was stirred at 20 °C for 1 h. LC-MS showed 3-(1-methyl-6-(piperidin-4-yl)-1H-indazol-3-yl)piperidine-2,6-dione was consumed completely and the desired mass was detected. The reaction mixture was filtered and the filtrate concentrated under high vacuum. The residue was purified by prep-TLC (SiO₂, Petroleum ether:Ethyl acetate=0:1, Rf=0.32) to give the title compound (300 mg, crude) as a white solid. LCMS (ES⁺): m/z 441.4 [M+H]⁺.

Synthesis of 2-(4-(3-(2,6-dioxopiperidin-3-yl)-1-methyl-1H-indazol-6-yl)piperidin-1-yl)acetic acid. To a solution of tert-butyl 2-(4-(3-(2,6-dioxopiperidin-3-yl)-1-methyl-1H-indazol-6-yl)piperidin-1-yl)acetate (190 mg, 431 μmol, 1.00 eq) in DCM (1 mL) was added TFA (245 mg, 2.16 mmol, 160 μL, 5.00 eq) at 20 °C. The mixture was stirred at 40 °C for 2 h. LC-MS showed tert-butyl 2-(4-(3-(2,6-dioxopiperidin-3-yl)-1-methyl-1H-indazol-6-yl)piperidin-1-yl)acetate was consumed completely and desired mass was detected. The reaction mixture was filtered and the filtrate concentrated under high vacuum to give the title compound (180 mg, crude) as a brown solid. LCMS (ES⁺): m/z 385.2 [M+H]⁺.

Synthesis of 3-(6-(1-(2-(4-(4-((5-chloro-4-(4′-fluoro-[1,1′-biphenyl]-3-yl)pyrimidin-2-yl)amino)piperidine-1-carbonyl)-4-hydroxypiperidin-1-yl)-2-oxoethyl)piperidin-4-yl)-1-methyl-1H-indazol-3-yl)piperidine-2,6-dione (Compound 290). To a solution of 2-(4-(3-(2,6-dioxopiperidin-3-yl)-1-methyl-1H-indazol-6-yl)piperidin-1-yl)acetic acid (180 mg, 468 μmol, 1.00 eq) in DMF (1 mL) was added HATU (178 mg, 468 μmol, 1.00 eq) and DIEA (181 mg, 1.40 mmol, 244 μL, 3.00 eq), [(4-((5-chloro-4-(4′-fluoro-[1,1′-biphenyl]-3-yl)pyrimidin-2-yl)amino)piperidin-1-yl)(4-hydroxypiperidin-4-yl)methanone (238 mg, 468 μmol, 1.00 eq) at 20 °C. The mixture was stirred at 20 °C for 1 h. LC-MS showed 2-(4-(3-(2,6-dioxopiperidin-3-yl)-1-methyl-1H-indazol-6-yl)piperidin-1-yl)acetic acid was consumed completely and the desired mass was detected. The reaction mixture was filtered and the filtrate concentrated under high vacuum. The residue was purified by prep-HPLC (Phenomenex luna C18 100*40 mm*5 um; mobile phase: [H₂O (0.2% FA)-ACN]; gradient: 25%-65% B over 8.0 min) to give the title compound (Compound 290) (29.3 mg, 32.9 μmol, 7.05% yield, 99.1% purity, 0.100 FA) as a white solid. LCMS (ES⁺): m/z 876.4 [M+H]⁺.

### Example 301. Synthesis of 3-(5-(1′-(3-(4-((5-chloro-4-(4′-fluoro-[1,1′-biphenyl]-3-yl)pyrimidin-2-yl)amino)piperidin-1-yl)-3-oxopropyl)-[1,4′-bipiperidin]-4-yl)-3-methyl-2-oxo-2,3-dihydro-1H-benzo[d]imidazol-1-yl)piperidine-2,6-dione (Compound 300)

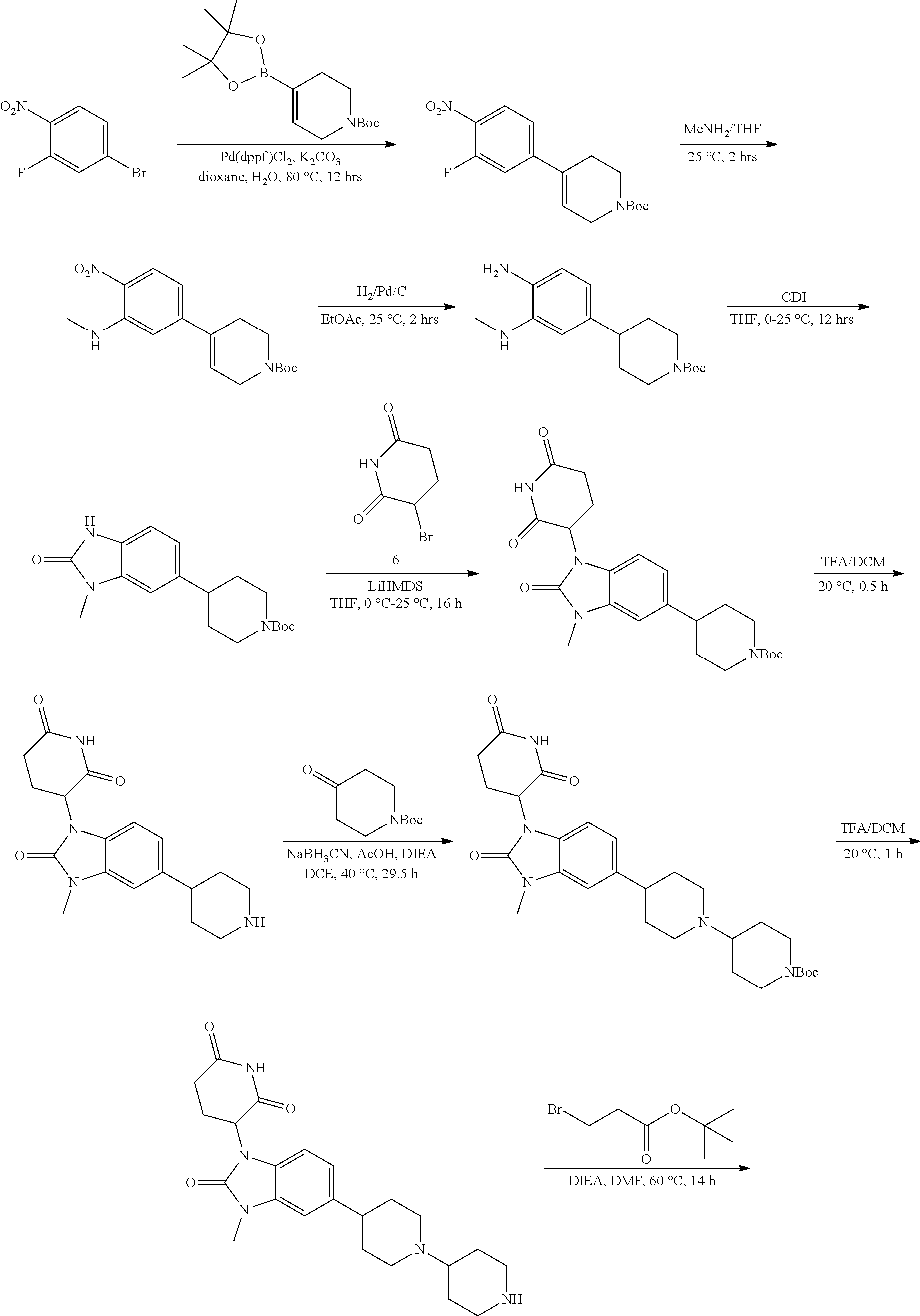

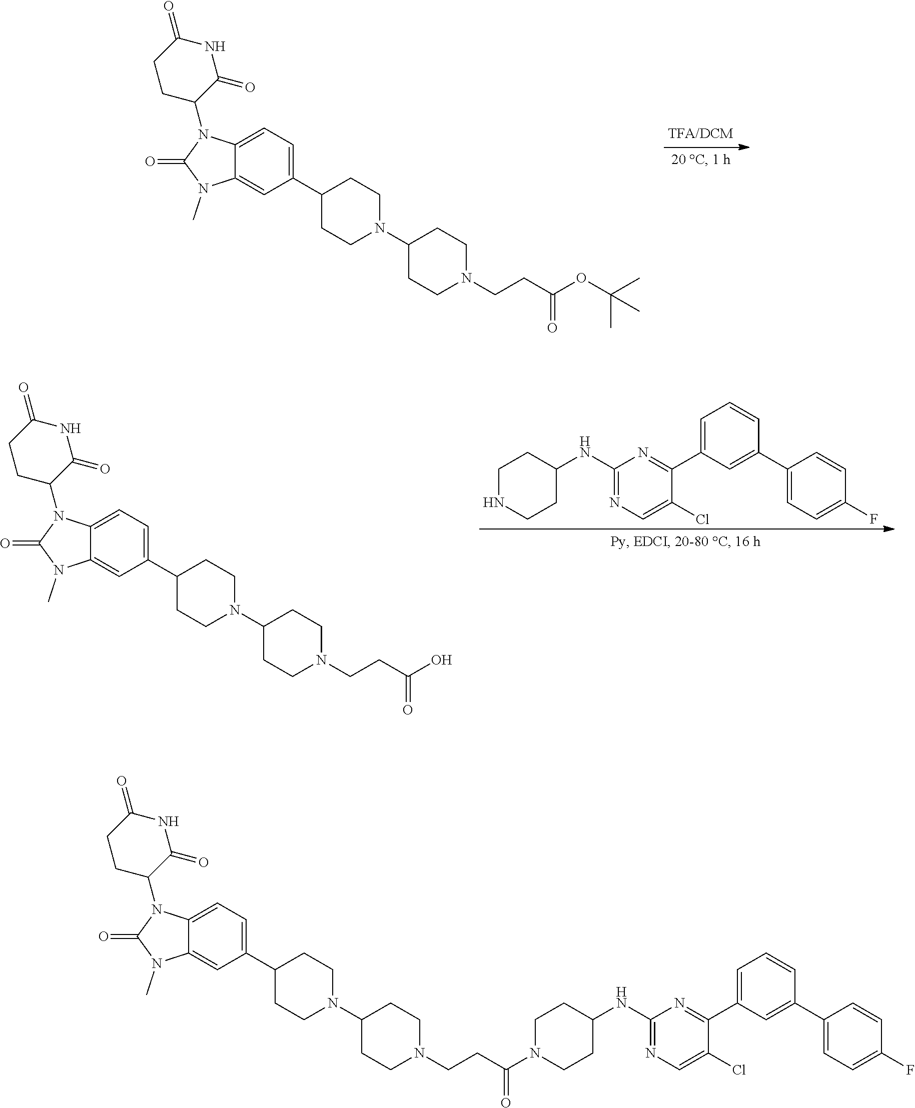 [SMILES: Cn1c(=O)n(C2CCC(=O)NC2=O)c2ccc(C3CCN(C4CCN(CCC(=O)OC(C)(C)C)CC4)CC3)cc21 ; Cn1c(=O)n(C2CCC(=O)NC2=O)c2ccc(C3CCN(C4CCN(CCC(=O)O)CC4)CC3)cc21 ; Fc1ccc(-c2cccc(-c3nc(NC4CCNCC4)ncc3Cl)c2)cc1 ; Cn1c(=O)n(C2CCC(=O)NC2=O)c2ccc(C3CCN(C4CCN(CCC(=O)N5CCC(Nc6ncc(Cl)c(-c7cccc(-c8ccc(F)cc8)c7)n6)CC5)CC4)CC3)cc21]

Synthesis of tert-butyl 4-(3-fluoro-4-nitrophenyl)-3,6-dihydropyridine-1(2H)-carboxylate. A mixture of 4-bromo-2-fluoro-1-nitrobenzene (355 g, 1617 mmol), tert-buty 4-(4,4,5,5-tetramethyl-1,3,2-dioxaborolan-2-yl)-3, 6-dihydro-2H-pyridine-1-carboxylate (500 g, 1617 mmol), Pd(dppf)Cl₂ (47.32 g, 64.7 mmol), K₂CO₃ (167 g, 1.21 mol) in dioxane (5000 mL) was degassed and purge with N₂ for 3 times, H₂O (500 mL) and then the mixture was stirred at 80 °C for 12 h under N₂ atmosphere. LC-MS showed the desired mass was detected. The reaction mixture was diluted with H₂O 4000 mL and extracted with Ethyl acetate 15000 mL (5000 mL*3). The combined organic layers were washed with brine 9000 mL (3000 mL*3), dried over Na₂SO₄, filtered and concentrated under reduced pressure to give the title compound (500 g, crude) as a brown solid. LCMS (ES⁺): m/z 223.0 [M−100+H]⁺.

Synthesis of tert-butyl 4-(3-methylamino-4-nitrophenyl)-3,6-dihydropyridine-1(2H)carboxylate. Two batches of this reaction was set up in parallel. To a solution of tert-butyl 4-(3-fluoro-4-nitrophenyl)-3,6-dihydropyridine-1(2H)-carboxylate (247 g, 766 mmol) in THF (1000 mL) was added MeNH₂ (2M, 988 mL). The mixture was stirred at 20 °C for 2 h. LC-MS showed the desired mass was detected. The reaction mixture was quenched by addition NH₄Cl 2000 mL at 0 °C, and then diluted with NaHCO₃ 2000 mL and extracted with EtOAc 6000 mL (2000 mL*3). The combined organic layers were washed with brine 6000 mL. (2000 mL*3), dried over Na₂SO₄, filtered and concentrated under reduced pressure to give the title compound (420 g, 1.26 mol, 82.20% yield) as a yellow solid. LCMS (ES⁺): m/z 334.2 [M+H]⁺

Synthesis of tert-butyl 4-(4-amino-3-(methylamino)phenyl)piperidine-1-carboxylate. Three batches of this reaction was set up in parallel. To a solution of tert-butyl 4-(3-(methylamino)-4-nitrophenyl)-3,6-dihydropyridine-1(2H)-carboxylate (140 g, 419.94 mmol, 1 eq) in EtOAc (2000 mL) was added Pd/C (10%, 15 g) under N₂ atmosphere. The suspension was degassed and purged with H₂ for 3 times. The mixture was stirred under H₂ (50 Psi) at 25 °C for 12 hours. LC-MS showed the desired mass was detected. The mixture was filtered through a Celite pad, and the filtrate was dried in vacuum to give a residue. The residue was triturated with MTBE (500 mL) at 25 °C for 60 min. The solid was collected by filtration, rinsed with MTBE (500 mL×3) and dried in vacuum to give the title compound (310 g, 78.63% yield, 97.6% purity) as a purple solid. LCMS (ES⁺): m/z 250.2 [M−56+H]⁺

Synthesis of tert-butyl 4-(3-methyl-2-oxo-2,3-dihydro-1H-benzo[d]imidazol-5-yl)piperidine-1-carboxylate. Three batches of this reaction was set up in parallel. To a solution of tert-butyl 4-(4-amino-3-(methylamino)phenyl)piperidine-1-carboxylate (103 g, 337 mmol) in THF (1500 mL) was added CDI (57.4 g, 354 mmol) at 0 °C. The mixture was stirred at 25 °C for 12 hours. LC-MS showed the desired mass was detected. The reaction mixture was concentrated under reduced pressure to give residue. The residue was triturated with MTBE (1000 mL) at 25 °C for 2 hours. The solid was collected by filtration, rinsed with MTBE 1800 mL (600 mL×3) and dried in vacuum to give the title compound (260 g, 77.54% yield) as a white solid. LCMS (ES⁺): m/z 332.3 [M+H]⁺

Synthesis of tert-butyl 4-(1-(2,6-dioxopiperidin-3-yl)-3-methyl-2-oxo-2,3-dihydro-1H-benzo[d]imidazol-5-yl)piperidine-1-carboxylate. Five batches of this reaction was set up in parallel. To a solution of tert-butyl 4-(3-methyl-2-oxo-2,3-dihydro-1H-benzo[d]imidazol-5-yl)piperidine-1-carboxylate (50 g, 150 mmol) in THF (1000 mL) was added LiHMDS (1 M, 452 mL) at 0 °C under N₂, and the mixture was stirred at 25 °C for 1 hour. Then the above mixture was added to 3-bromopiperidine-2,6-dione (49.3 g, 256 mmol, 1.7 eq) under N₂. The reaction mixture was stirred at 25 °C for 12 hours. LC-MS showed the desired mass was detected. The reaction mixture was quenched by addition sat.aq.NH₄Cl 500 mL at 0 °C, and then diluted with H₂O 500 mL and extracted with EtOAc 900 mL (300 mL*3). The combined organic layers were washed with brine 900 mL (300 mL*3), dried over Na₂SO₄ filtered and concentrated under reduced pressure to give a residue. The residue was purified by column chromatography (SiO₂, dichloromethane/Ethyl acetate=1/0 to 1/1) to give the title compound (100 g, 74.89% yield) as a white solid. LCMS (ES⁺): m/z 387.3 [M−56+H]⁺

Synthesis of 3-(3-methyl-2-oxo-5-(piperidin-4-yl)-2,3-dihydro-1H-benzo[d]imidazol-1-yl)piperidine-2,6-dione. A solution of tert-butyl 4-(1-(2,6-dioxopiperidin-3-yl)-3-methyl-2-oxo-2,3-dihydro-1H-benzo[d]imidazol-5-yl)piperidine-1-carboxylate (500 mg, 1.13 mmol, 1.00 eq) in DCM (6 mL) and TFA (2 mL) was stirred at 20 °C for 0.5 h. LCMS showed tert-butyl 4-(1-(2,6-dioxopiperidin-3-yl)-3-methyl-2-oxo-2,3-dihydro-1H-benzo[d]imidazol-5-yl)piperidine-1-carboxylate was consumed and the desired target MS was detected. The mixture was concentrated under reduced pressure to give the title compound (515 mg, crude, TFA) as a yellow oil. LCMS (ES⁺): m/z 343.1 [M+H]⁺.

Synthesis of tert-butyl 4-(1-(2,6-dioxopiperidin-3-yl)-3-methyl-2-oxo-2,3-dihydro-1H-benzo[d]imidazol-5-yl)-[1,4′-bipiperidine]-1′-carboxylate. To a solution of 3-(3-methyl-2-oxo-5-(piperidin-4-yl)-2,3-dihydro-1H-benzo[d]imidazol-1-yl)piperidine-2,6-dione (1.00 g, 2.19 mmol, 1.00 eq, TFA) in DCE (80 mL) was added DIEA to adjust pH to 8 at 20 °C. The mixture was stirred at 20 °C for 1 h. Then AcOH (395 mg, 6.57 mmol, 376 μL, 3.00 eq) was added to the mixture at 20 °C, pH was 5 now. The mixture was stirred at 20 °C for 30 min. Then tert-butyl 4-oxopiperidine-1-carboxylate (5.24 g, 26.3 mmol, 12.0 eq) was added to the mixture at 20 °C. The mixture was stirred at 40 °C for 1 h. Then NaBH₃CN (2.07 g, 32.9 mmol, 15.0 eq) was added to the mixture at 20 °C. The mixture was stirred at 40 °C for 12 h. LC-MS showed ~54% of 3-(3-methyl-2-oxo-5-(piperidin-4-yl)-2,3-dihydro-1H-benzo[d]imidazol-1-yl)piperidine-2,6-dione was remaining and the desired mass was detected. Then tert-butyl 4-oxopiperidine-1-carboxylate (5.24 g, 26.3 mmol, 12.0 eq) was added to the mixture at 20 °C. The mixture was stirred at 40 °C for 3 h. Then NaBH₃CN (2.07 g, 32.9 mmol, 15.0 eq) was added to the mixture. The mixture was stirred at 40 °C for 12 h. LC-MS showed 3-(3-methyl-2-oxo-5-(piperidin-4-yl)-2,3-dihydro-1H-benzo[d]imidazol-1-yl)piperidine-2,6-dione was remaining little and the desired mass was detected. The reaction mixture was concentrated under reduced pressure to remove solvent. Water (80 mL) was added to the mixture at 20 °C. Then the reaction mixture was extracted with EtOAc (80 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=1:1 to 1:5) to give the title compound (1.05 g, 2.00 mmol, 91.2% yield) as a white solid. LCMS (ES⁺): m/z 526.3 [M+H]⁺.

Synthesis of 3-(5-([1,4′-bipiperidin]-4-yl)-3-methyl-2-oxo-2,3-dihydro-1H-benzo[d]imidazol-1-yl)piperidine-2,6-dione. To a solution of tert-butyl 4-(1-(2,6-dioxopiperidin-3-yl)-3-methyl-2-oxo-2,3-dihydro-1H-benzo[d]imidazol-5-yl)-[1,4′-bipiperidine]-1′-carboxylate (666 mg, 1.27 mmol, 1.00 eq) in DCM (9 mL) was added TFA (4.65 g, 40.8 mmol, 3.00 mL, 32.2 eq) at 20 °C. The mixture was stirred at 20 °C for 1 h. LC-MS showed tert-butyl 4-(1-(2,6-dioxopiperidin-3-yl)-3-methyl-2-oxo-2,3-dihydro-1H-benzo[d]imidazol-5-yl)-[1,4′-bipiperidine]-1′-carboxylate was consumed completely and the desired mass was detected. The reaction mixture was concentrated under reduced pressure to give the title compound (680 mg, crude, TFA) as a yellow oil. LCMS (ES⁺): m/z 426.3 [M+H]⁺.

Synthesis of tert-butyl 3-(4-(1-(2,6-dioxopiperidin-3-yl)-3-methyl-2-oxo-2,3-dihydro-1H-benzo[d]imidazol-5-yl)-[1,4′-bipiperidin]-1′-yl)propanoate. To a solution of 3-(5-([1,4′-bipiperidin]-4-yl)-3-methyl-2-oxo-2,3-dihydro-1H-benzo[d]imidazol-1-yl)piperidine-2,6-dione (680 mg, 1.26 mmol, 1.00 eq, TFA) in DMF (5 mL) were added DIEA (652 mg, 5.04 mmol, 878 μL, 4.00 eq) and tert-butyl 3-bromopropanoate (791 mg, 3.78 mmol, 631 μL, 3.00 eq) at 20 °C. The mixture was stirred at 60 °C for 2 h. LC-MS showed (5-([1,4′-bipiperidin]-4-yl)-3-methyl-2-oxo-2,3-dihydro-1H-benzo[d]imidazol-1-yl)piperidine-2,6-dione was remaining, and the desired mass was detected. Then the mixture was stirred at 60 °C for another 12 h. LCMS showed (5-([1,4′-bipiperidin]-4-yl)-3-methyl-2-oxo-2,3-dihydro-1H-benzo[d]imidazol-1-yl)piperidine-2,6-dione was remaining little, and the desired mass was detected. The reaction mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 1%-30% B over 8.0 min) to give the title compound (83.0 mg, 150 μmol, 11.9% yield) as a white solid. LCMS (ES⁺): m/z 554.4 [M+H]⁺.

Synthesis of 3-(4-(1-(2,6-dioxopiperidin-3-yl)-3-methyl-2-oxo-2,3-dihydro-1H-benzo[d]imidazol-5-yl)-[1,4′-bipiperidin]-1′-yl)propanoic acid. To a solution of tert-butyl 3-(4-(1-(2,6-dioxopiperidin-3-yl)-3-methyl-2-oxo-2,3-dihydro-1H-benzo[d]imidazol-5-yl)-[1,4′-bipiperidin]-1′-yl)propanoate (83.0 mg, 150 μmol, 1.00 eq) in DCM (3 mL) was added TFA (1.54 g, 13.6 mmol, 1 mL, 89.8 eq) at 20 °C. The mixture was stirred at 20 °C for 1 h. LC-MS showed tert-butyl 3-(4-(1-(2,6-dioxopiperidin-3-yl)-3-methyl-2-oxo-2,3-dihydro-1H-benzo[d]imidazol-5-yl)-[1,4′-bipiperidin]-1′-yl)propanoate was consumed completely and the desired mass was detected. The reaction mixture was concentrated under reduced pressure to give the title compound (74.0 mg, crude) as a yellow oil. LCMS (ES⁺): m/z 498.3 [M+H]⁺.

Synthesis of 3-(5-(1′-(3-(4-((5-chloro-4-(4′-fluoro-[1,1′-biphenyl]-3-yl)pyrimidin-2-yl)amino)piperidin-1-yl)-3-oxopropyl)-[1,4′-bipiperidin]-4-yl)-3-methyl-2-oxo-2,3-dihydro-1H-benzo[d]imidazol-1-yl)piperidine-2,6-dione (Compound 300). To a solution of 3-(4-(1-(2,6-dioxopiperidin-3-yl)-3-methyl-2-oxo-2,3-dihydro-1H-benzo[d]imidazol-5-yl)-[1,4′-bipiperidin]-1′-yl)propanoic acid (74.0 mg, 149 μmol, 1.00 eq) in Py (1 mL) were added 5-chloro-4-(4′-fluoro-[1,1′-biphenyl]-3-yl)-N-(piperidin-4-yl)pyrimidin-2-amine (44.3 mg, 89.2 μmol, 0.600 eq, TFA) and EDCI (57.0 mg, 297 μmol, 2.00 eq) at 20 °C. The mixture was stirred at 20 °C for 1 h. LC-MS showed 3-(4-(1-(2,6-dioxopiperidin-3-yl)-3-methyl-2-oxo-2,3-dihydro-1H-benzo[d]imidazol-5-yl)-[1,4′-bipiperidin]-1′-yl)propanoic acid was remaining most, and the desired mass was detected. Then the mixture was stirred at 60 °C for another 12 h. LC-MS showed 3-(4-(1-(2,6-dioxopiperidin-3-yl)-3-methyl-2-oxo-2,3-dihydro-1H-benzo[d]imidazol-5-yl)-[1,4′-bipiperidin]-1′-yl)propanoic acid was remaining, and the desired mass was detected. Then the mixture was stirred at 80 °C for another 3 h. LC-MS showed 3-(4-(1-(2,6-dioxopiperidin-3-yl)-3-methyl-2-oxo-2,3-dihydro-1H-benzo[d]imidazol-5-yl)-[1,4′-bipiperidin]-1′-yl)propanoic acid was consumed completely and the desired mass was detected. The reaction mixture was concentrated under reduced pressure to remove solvent. The residue was purified by prep-HPLC (column: Phenomenex luna C18 100*40 mm*5 um; mobile phase: [H₂O (0.2% FA)-ACN]; gradient: 15%-50% B over 8.0 min) to give the title compound (Compound 300) (6.60 mg, 7.58 μmol, 5.10% yield, 99.1% purity) as a white solid. LCMS (ES⁺): m/z 862.4 [M+H]⁺.

### Example 302. Synthesis of trans-3-(5-(1-(2-(4-(4-((5-chloro-4-(4′-fluoro-[1,1′-biphenyl]-3-yl)pyrimidin-2-yl)amino)piperidine-1-carbonyl)cyclohexyl)ethyl)piperidin-4-yl)-3-methyl-2-oxo-2,3-dihydro-1H-benzo[d]imidazol-1-yl)piperidine-2,6-dione (Compound 338)

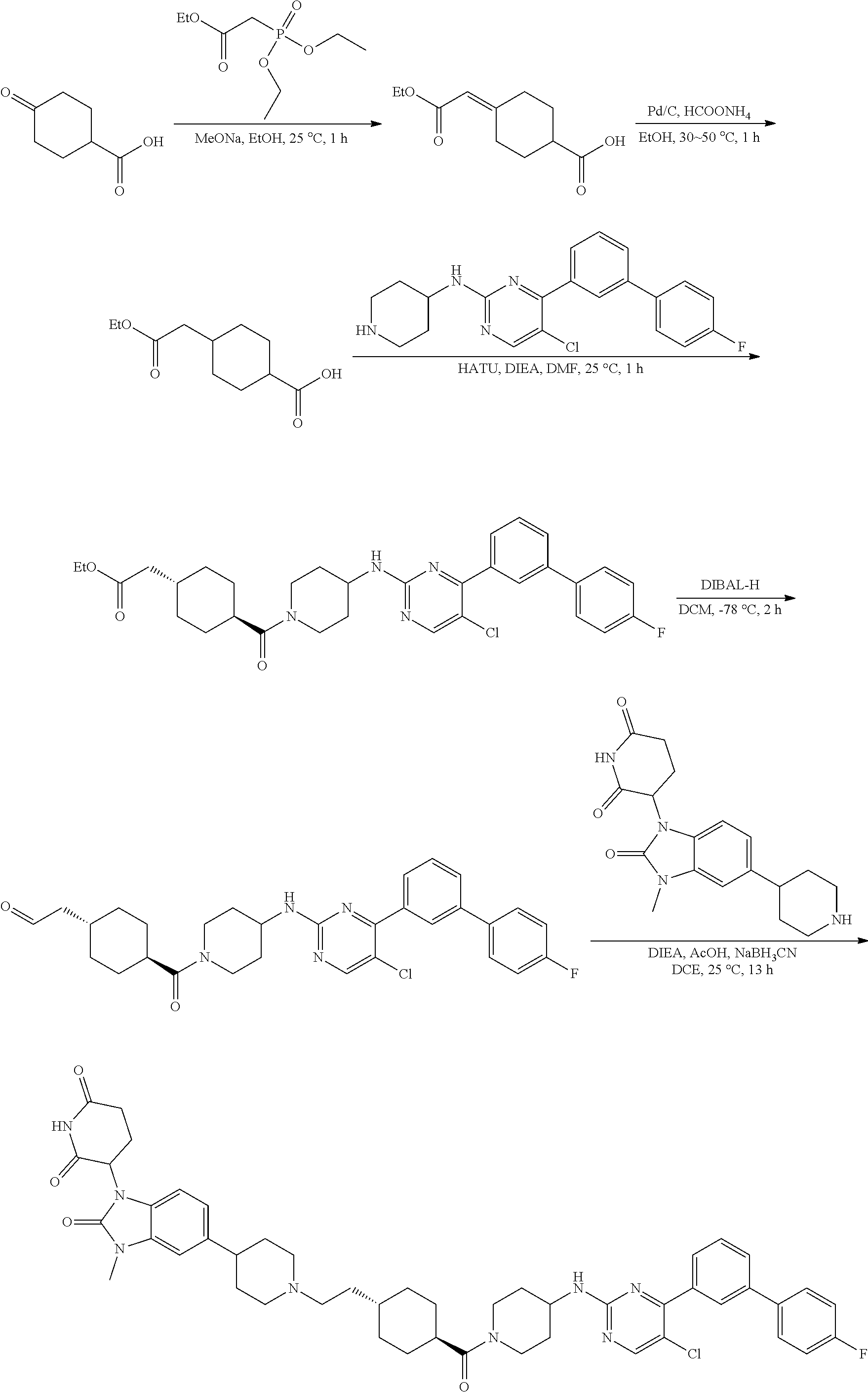

Synthesis of 4-(2-ethoxy-2-oxoethylidene)cyclohexane-1-carboxylic acid. To a solution of 4-oxocyclohexanecarboxylic acid (10.0 g, 70.3 mmol, 1.00 eq) in EtOH (400 mL) was added sodium methoxide (4.56 g, 84.4 mmol, 1.20 eq) followed by ethyl 2-diethoxyphosphorylacetate (17.3 g, 77.3 mmol, 15.35 mL, 1.10 eq) at 25 °C under nitrogen atmosphere. The reaction mixture was cooled in an ice bath to 0 °C and sodium; methanolate (4.18 g, 77.3 mmol, 1.10 eq) was subsequently added. After stirring for 1 hour at 25 °C under nitrogen atmosphere. LC-MS showed the desired mass was detected. The reaction mixture was brought up to room temperature and adjusted to pH=5 with glacial acetic acid (3 ml). The acidified mixture was concentrated and the remaining oil was partitioned between ethyl acetate (100 ml) and 1 M hydrochloric acid in water (100 mL). The organic phase was separated, washed with water (100 mL) twice, dried over anhydrous Na₂SO₄, filtered and concentrated under reduced pressure to give the title compound (14 g, crude) as a yellow oil. LCMS (ES⁺): m/z 211.1 [M−H]⁺.

Synthesis of 4-(2-ethoxy-2-oxoethyl)cyclohexane-1-carboxylic acid. The reaction was set up in two batches. To a solution of 4-(2-ethoxy-2-oxoethylidene)cyclohexane-1-carboxylic acid (3.50 g, 16.4 mmol, 1.00 eq) in EtOH (100 mL) was added 10% Pd/C (350 mg, 50.0% purity,) at 25 °C. Ammonium formate (2.60 g, 41.2 mmol, 2.50 eq) was subsequently added at 30 °C. The mixture was stirred at 50 °C for 1 hour. One batch in parallel was monitored in random. LC-MS showed the desired mass was detected. Two in parallel batches were combined and the mixture was filtered and concentrated under reduced pressure to give a residue. The mixture was dissolved in Ethyl acetate (50 mL) and 1 N HCl (50 mL). The organic layer was separated and washed with water 50 mL, dried over Na₂SO₄, filtered and concentrated under reduced pressure to give the title compound (5.00 g, crude) as a brown oil. LCMS (ES⁺): m/z 215.1 [M+H]⁺.

Synthesis of trans-ethyl 2-(4-(4-((5-chloro-4-(4′-fluoro-[1,1′-biphenyl]-3-yl)pyrimidin-2-yl)amino)piperidine-1-carbonyl)cyclohexyl)acetate. To a solution of 4-(2-ethoxy-2-oxoethyl)cyclohexane-1-carboxylic acid (2.00 g, 9.33 mmol, 1.00 eq) in DMF (6 mL) was added DIEA (1.21 g, 9.33 mmol, 1.63 mL, 1.00 eq) until pH~7. To a solution of 5-chloro-4-[3-(4-fluorophenyl)phenyl]-N-(4-piperidyl)pyrimidin-2-amine (1.79 g, 4.67 mmol, 0.500 eq) in DMF (10 mL) was added DIEA (1.21 g, 9.33 mmol, 1.63 mL, 1.00 eq) until pH~7. Then HATU (1.77 g, 4.67 mmol, 0.500 eq) was added to the mixture of the two solutions. The mixture was stirred at 25 °C for 1 h. LC-MS showed the desired mass was detected. The reaction solution was diluted with water (30 mL), and extracted with EtOAc (30 mL*3). The combined organic layers were washed with brine (50 mL), dried over Na₂SO₄, filtered and concentrated under reduced pressure. The residue was purified by column chromatography (SiO₂, Petroleum ether:Ethyl acetate=1:0 to 1:1). The crude product was obtained as a yellow oil. The crude product was purified by SFC (column: DAICEL CHIRALCEL OJ (250 mm*30 mm, 10 um); mobile phase: [CO₂-EtOH (0.1% NH₃H₂O)]; B %: 65%, isocratic elution mode) to give the title compound (700 mg, 1.14 mmol, 12.1% yield, 94.0% purity) as a colorless gum. LCMS (ES⁺): m/z 579.3 [M+H]⁺. and cis-ethyl 2-(4-(4-((5-chloro-4-(4′-fluoro-[1,1′-biphenyl]-3-yl)pyrimidin-2-yl)amino)piperidine-1-carbonyl)cyclohexyl)acetate (700 mg, 1.17 mmol, 12.56% yield, 97% purity) as a yellow oil. LCMS (ES⁺): m/z 579.3 [M+H]⁺.

Synthesis of trans-2-(4-(4-((5-chloro-4-(4′-fluoro-[1,1′-biphenyl]-3-yl)pyrimidin-2-yl)amino)piperidine-1-carbonyl)cyclohexyl)acetaldehyde. To a solution of trans-ethyl 2-(4-(4-((5-chloro-4-(4′-fluoro-[1,1′-biphenyl]-3-yl)pyrimidin-2-yl)amino)piperidine-1-carbonyl)cyclohexyl)acetate (300 mg, 518 μmol, 1.00 eq) in DCM (3 mL) was added DIBAL-H (1 M, 777 μL, 1.50 eq) at −78 °C under N₂ atmosphere. The mixture was stirred at −78 °C for 2 h. LC-MS showed desired mass was detected. The reaction mixture was quenched by addition water 5 mL at 0 °C under N₂, and extracted with DCM mL (5 mL*3). The combined organic layers were washed with brine mL (5 mL*3), dried over Na₂SO₄, filtered and concentrated under reduced pressure. The crude product (100 mg, crude) was obtained as a yellow liquid. The crude product was used into the next step without further purification. LCMS (ES⁺): m/z 535.3 [M+H]⁺.

Synthesis of trans-3-(5-(1-(2-(4-(4-((5-chloro-4-(4′-fluoro-[1,1′-biphenyl]-3-yl)pyrimidin-2-yl)amino)piperidine-1-carbonyl)cyclohexyl)ethyl)piperidin-4-yl)-3-methyl-2-oxo-2,3-dihydro-1H-benzo[d]imidazol-1-yl)piperidine-2,6-dione (Compound 338). To a solution of 3-[3-methyl-2-oxo-5-(4-piperidyl)benzimidazol-1-yl]piperidine-2,6-dione (63.9 mg, 186 μmol, 1.00 eq) in DCE (3 mL) was added DIEA (72.4 mg, 560 μmol, 97.6 μL, 3.00 eq) at 25 °C. The mixture was stirred at 25 °C for 0.5 h. Then AcOH (56.1 mg, 934 μmol, 53.5 μL, 5.00 eq) and trans-2-(4-(4-((5-chloro-4-(4′-fluoro-[1,1′-biphenyl]-3-yl)pyrimidin-2-yl)amino)piperidine-1-carbonyl)cyclohexyl)acetaldehyde (100 mg, 186 μmol, 1.00 eq) were added at 25 °C. The mixture was stirred at 25 °C for 0.5 h. Then NaBH₃CN (58.7 mg, 934 mol, 5.00 eq) was added at 25 °C. The mixture was stirred at 25 °C for 12 h. LC-MS showed desired mass was detected. The reaction mixture was diluted with H₂O 20 mL and extracted with EtOAc (30 mL*3). The combined organic layers were concentrated under reduced pressure. The residue was purified by prep-HPLC (column: Phenomenex luna C18 100*40 mm*3 um; mobile phase: [H₂O (0.1% TFA)-ACN]; gradient: 25%-55% B over 8.0 min). (column: Phenomenex Gemini NX-C18 (75*30 mm*3 um); mobile phase: [H₂O (0.05% NH₃H₂O+10 mM NH₄HCO₃)-ACN]; gradient: 45%-85% B over 8.0 min) to give the title compound (Compound 338) (2.40 mg, 2.71 μmol, 1.45% yield, 97.3% purity) as a white solid. LCMS (ES⁺): m/z 861.4[M+H]⁺.

### Example 303. Synthesis of 3-(6-(1-((1-(2-(4-((5-chloro-4-(4′-fluoro-[1,1′-biphenyl]-3-yl)pyrimidin-2-yl)amino)piperidin-1-yl)-2-oxoethyl)piperidin-4-yl)methyl)piperidin-4-yl)-2-oxobenzo[d]oxazol-3 (2H)-yl)piperidine-2,6-dione (Compound 294)

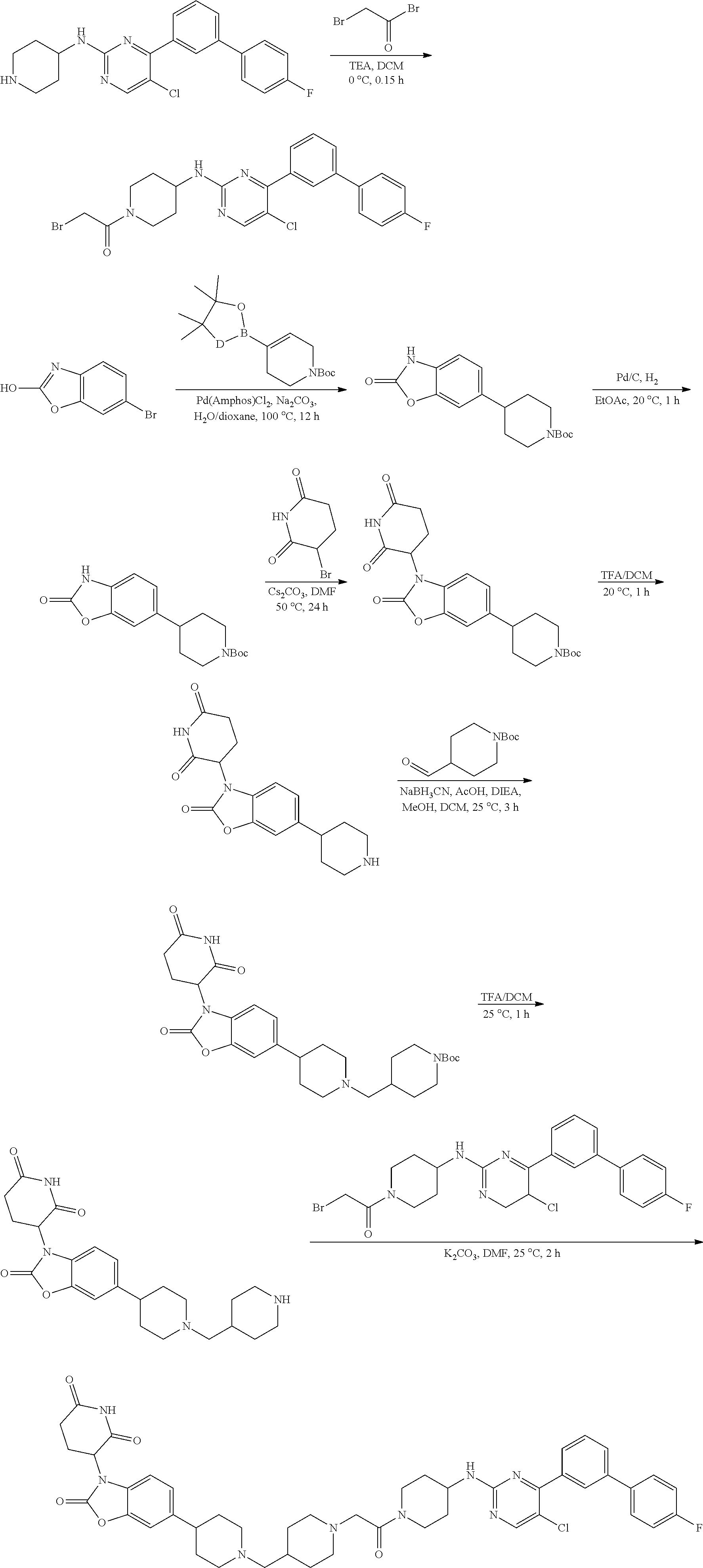

Synthesis of 2-bromo-1-(4-((5-chloro-4-(4′-fluoro-[1,1′-biphenyl]-3-yl)pyrimidin-2-yl)amino)piperidin-1-yl)ethan-1-one. To a solution of 5-chloro-4-(4′-fluoro-[1,1′-biphenyl]-3-yl)-N-(piperidin-4-yl)pyrimidin-2-amine (900 mg, 2.35 mmol, 1 eq) in DCM (2 mL) were added TEA (713.60 mg, 7.05 mmol, 981.57 μL, 3 eq) and 2-bromoacetyl bromide (474.48 mg, 2.35 mmol, 204.78 μL, 1 eq) at 0 °C. The mixture was stirred at 0 °C for 0.15 h. LCMS showed 5-chloro-4-(4′-fluoro-[1,1′-biphenyl]-3-yl)-N-(piperidin-4-yl)pyrimidin-2-amine was consumed completely and the desired mass was detected. The reaction mixture was concentrated under reduced pressure to give a residue. The residue was purified by column chromatography (SiO₂, Petroleum ether:Ethyl acetate=5:1 to 2:1) to give the title compound (750 mg, 1.33 mmol, 56.78% yield, 89.66% purity) as a white solid. LCMS (ES⁺): m/z 503.1 [M+H]⁺.

Synthesis of tert-butyl 4-(2-oxo-2,3-dihydrobenzo[d]oxazol-6-yl)-3,6-dihydropyridine-1(2H)-carboxylate. To a solution of 6-bromobenzo[d]oxazol-2-ol (6.00 g, 28.0 mmol, 1.00 eq) and tert-butyl 4-(4,4,5,5-tetramethyl-1,3,2-dioxaborolan-2-yl)-3,6-dihydro-2H-pyridine-1-carboxylate (10.4 g, 33.6 mmol, 1.20 eq) in dioxane (120 mL) and H₂O (40 mL) were added Na₂CO₃ (7.43 g, 70.1 mmol, 2.50 eq) and Pd(Amphos)Cl₂ (1.99 g, 2.80 mmol, 1.99 mL, 0.100 eq) at 20 °C under N₂ atmosphere. The mixture was stirred at 100 °C for 12 h under N. LCMS showed the desired mass was detected. Water (80 mL) was added to the mixture at 20 °C. Then the reaction mixture was extracted with EtOAc (80 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=2:1 to 1:1) to give the title compound (6.70 g, 21.2 mmol, 75.5% yield) as a yellow oil. LCMS (ES⁺): m/z 261.1 [M+H−56]⁺.

Synthesis of tert-butyl 4-(2-oxo-2,3-dihydrobenzo[d]oxazol-6-yl)piperidine-1-carboxylate. To a solution of tert-butyl 4-(2-oxo-2,3-dihydrobenzo[d]oxazol-6-yl)-3,6-dihydropyridine-1(2H)-carboxylate (6.70 g, 21.2 mmol, 1.00 eq) in EtOAc (100 mL) was added 10% Pd/C (7.00 g, 50% purity) at 20 °C under H₂ (15 psi) atmosphere. The mixture was stirred at 20 °C for 1 h under H₂. LCMS showed tert-butyl 4-(2-oxo-2,3-dihydrobenzo[d]oxazol-6-yl)-3,6-dihydropyridine-1(2H)-carboxylate was consumed completely and the desired mass was detected. The reaction mixture was filtered and the filtrate was concentrated under reduced pressure to give the title compound (7.70 g, crude) as a yellow oil. LCMS (ES⁺): m/z 263.1 [M+H−56]⁺

Synthesis of tert-butyl 4-(3-(2,6-dioxopiperidin-3-yl)-2-oxo-2,3-dihydrobenzo[d]oxazol-6-yl)piperidine-1-carboxylate. To a solution of tert-butyl 4-(2-oxo-2,3-dihydrobenzo[d]oxazol-6-yl)piperidine-1-carboxylate (7.70 g, 24.2 mmol, 1.00 eq) in DMF (140 mL) were added Cs₂CO₃ (15.8 g, 48.4 mmol, 2.00 eq) and 3-bromopiperidine-2,6-dione (6.97 g, 36.3 mmol, 1.50 eq) at 20 °C. The mixture was stirred at 50 °C for 12 h. LCMS showed tert-butyl 4-(2-oxo-2,3-dihydrobenzo[d]oxazol-6-yl)piperidine-1-carboxylate was remaining and the desired mass was detected. 3-bromopiperidine-2,6-dione (3.00 g, 15.6 mmol, 0.646 eq) was added to the mixture at 20 °C. The mixture was stirred at 50 °C for another 12 h. LCMS showed tert-butyl 4-(2-oxo-2,3-dihydrobenzo[d]oxazol-6-yl)piperidine-1-carboxylate was consumed completely and the desired mass was detected. Water (130 mL) was added to the mixture at 25 °C. The solid was precipitated. It was filtered and the filter cake was dried under reduced pressure to give the title compound (12.5 g, crude) as a green solid. LCMS (ES⁺): m/z 374.2 [M+H−56]⁺

Synthesis of 3-(2-oxo-6-(piperidin-4-yl)benzo[d]oxazol-3 (2H)-yl)piperidine-2,6-dione. To a solution of tert-butyl 4-(3-(2,6-dioxopiperidin-3-yl)-2-oxo-2,3-dihydrobenzo[d]oxazol-6-yl)piperidine-1-carboxylate (3.00 g, 6.99 mmol, 1.00 eq) in DCM (21 mL) was added TFA (7 mL) at 20 °C. The mixture was stirred at 20 °C for 1 h. LCMS showed tert-butyl 4-(3-(2,6-dioxopiperidin-3-yl)-2-oxo-2,3-dihydrobenzo[d]oxazol-6-yl)piperidine-1-carboxylate was consumed completely and the desired mass was detected. The reaction mixture was concentrated under reduced pressure to give the title compound (3.10 g, crude, TFA) as a green oil. LCMS (ES⁺): m/z 330.2 [M+H]⁺.

Synthesis of tert-butyl 4-((4-(3-(2,6-dioxopiperidin-3-yl)-2-oxo-2,3-dihydrobenzo[d]oxazol-6-yl)piperidin-1-yl)methyl)piperidine-1-carboxylate. To a solution of 3-(2-oxo-6-(piperidin-4-yl)benzo[d]oxazol-3 (2H)-yl)piperidine-2,6-dione (500 mg, 1.13 mmol, 1.00 eq, TFA) in DCM (5 mL) and MeOH (5 mL) was added DIEA dropwise to adjust pH to 8. The mixture was stirred at 25 °C for 0.5 h. Then tert-butyl 4-formylpiperidine-1-carboxylate (361 mg, 1.69 mmol, 1.50 eq) and AcOH (67.7 mg, 1.13 mmol, 64.6 μL, 1.00 eq) were added to the reaction to the mixture at 25 °C, pH was 5-6 now. The mixture was stirred at 25 °C for 0.5 h. NaBH₃CN (213 mg, 3.38 mmol, 3.00 eq) was added to the mixture at 25 °C. The mixture was stirred at 25 °C for 2 h. LCMS showed 3-(2-oxo-6-(piperidin-4-yl)benzo[d]oxazol-3 (2H)-yl)piperidine-2,6-dione was consumed completely and the desired mass was detected. Then the reaction mixture was concentrated under reduced pressure to give a residue. The residue was purified by prep-TLC (SiO₂, Dichloromethane:Methanol=10:1) to give the title compound (390 mg, 580 μmol, 51.4% yield, 78.3% purity) as a white solid. LCMS (ES⁺): m/z 527.5 [M+H]⁺.

Synthesis of 3-(2-oxo-6-(1-(piperidin-4-ylmethyl)piperidin-4-yl)benzo[d]oxazol-3 (2H)-yl)piperidine-2,6-dione. To a solution of tert-butyl 4-((4-(3-(2,6-dioxopiperidin-3-yl)-2-oxo-2,3-dihydrobenzo[d]oxazol-6-yl)piperidin-1-yl)methyl)piperidine-1-carboxylate (300 mg, 570 μmol, 1.00 eq) in DCM (3 mL) was added TFA (1 mL) at 25 °C. The mixture was stirred at 25 °C for 12 h. LCMS showed tert-butyl 4-((4-(3-(2,6-dioxopiperidin-3-yl)-2-oxo-2,3-dihydrobenzo[d]oxazol-6-yl)piperidin-1-yl)methyl)piperidine-1-carboxylate was consumed completely and the desired mass was detected. The reaction mixture was concentrated under reduced pressure to give the title compound (300 mg, crude, TFA) as a yellow oil. LCMS (ES⁺): m/z 427.3 [M+H]⁺.

Synthesis of 3-(6-(1-((1-(2-(4-((5-chloro-4-(4′-fluoro-[1,1′-biphenyl]-3-yl)pyrimidin-2-yl)amino)piperidin-1-yl)-2-oxoethyl)piperidin-4-yl)methyl)piperidin-4-yl)-2-oxobenzo[d]oxazol-3 (2H)-yl)piperidine-2,6-dione (Compound 294). To a solution of 3-(2-oxo-6-(1-(piperidin-4-ylmethyl)piperidin-4-yl)benzo[d]oxazol-3 (2H)-yl)piperidine-2,6-dione (200 mg, 370 μmol, 1.00 eq, TFA) in DMF (4 mL) were added K₂CO₃ (153 mg, 1.11 mmol, 3.00 eq) and 2-bromo-1-[4-[[5-chloro-4-[3-(4-fluorophenyl)phenyl]pyrimidin-2-yl]amino]-1-piperidyl]ethanone (186 mg, 370 μmol, 1.00 eq) at 25 °C. The mixture was stirred at 25 °C for 1 h. LCMS showed 3-(2-oxo-6-(1-(piperidin-4-ylmethyl)piperidin-4-yl)benzo[d]oxazol-3 (2H)-yl)piperidine-2,6-dione remained and the desired mass was detected. The mixture was stirred at 25 °C for another 1 h. LCMS showed 3-(2-oxo-6-(1-(piperidin-4-ylmethyl)piperidin-4-yl)benzo[d]oxazol-3 (2H)-yl)piperidine-2,6-dione was consumed completely and the desired mass was detected. The reaction mixture was concentrated under reduced pressure to give a residue. The residue were purified by prep-HPLC (column: Phenomenex Luna C18 100*30 mm*3 um; mobile phase: [H₂O (0.2% FA)-ACN]; gradient: 10%-40% B over 8.0 min) to give the title compound (Compound 294) (33.1 mg, 37.0 μmol, 9.99% yield, 100% purity, FA) as a white solid. LCMS (ES⁺): m/z 849.4 [M+H]⁺.

### Example 304. Synthesis of 3-(6-(1-((1-(2-(4-((5-chloro-4-(4′-fluoro-[1,1′-biphenyl]-3-yl)pyrimidin-2-yl)amino)piperidin-1-yl)-2-oxoethyl)piperidin-4-yl)methyl)piperidin-4-yl)-1-methyl-1H-indazol-3-yl)piperidine-2,6-dione (Compound 277)

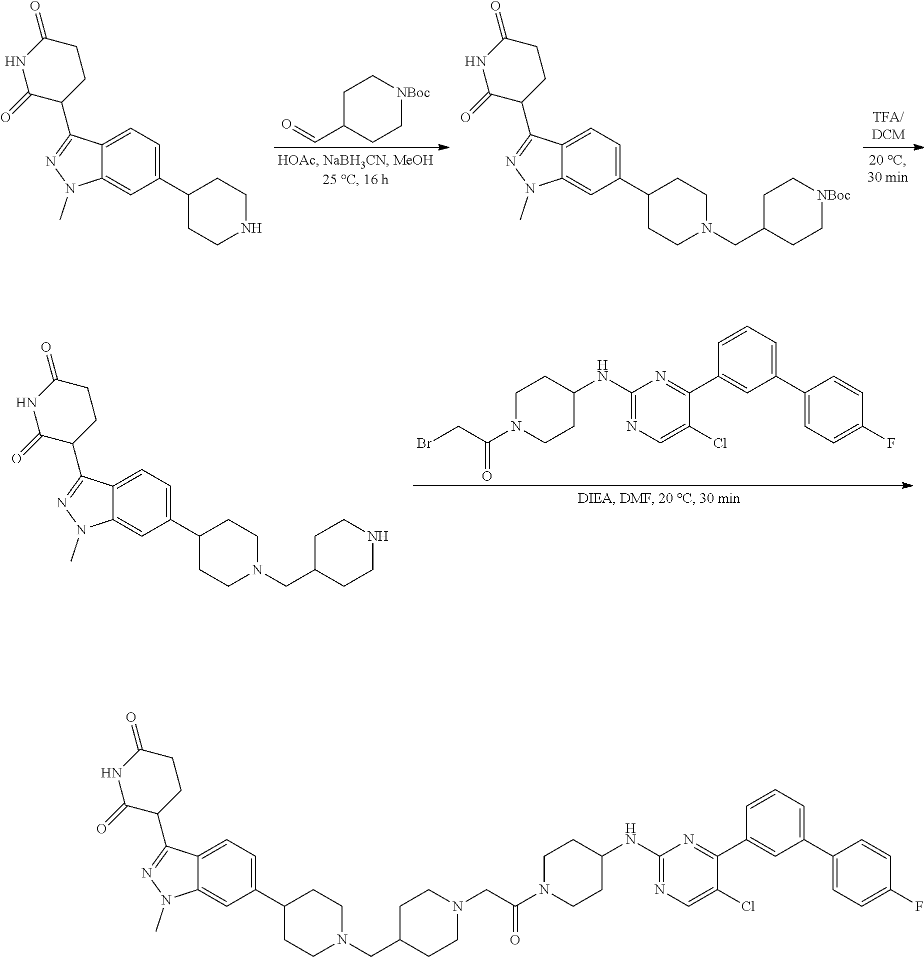 [SMILES: Cn1nc(C2CCC(=O)NC2=O)c2ccc(C3CCNCC3)cc21 ; CC(C)(C)OC(=O)N1CCC(C=O)CC1 ; Cn1nc(C2CCC(=O)NC2=O)c2ccc(C3CCN(CC4CCN(C(=O)OC(C)(C)C)CC4)CC3)cc21 ; Cn1nc(C2CCC(=O)NC2=O)c2ccc(C3CCN(CC4CCNCC4)CC3)cc21 ; O=C(CBr)N1CCC(Nc2ncc(Cl)c(-c3cccc(-c4ccc(F)cc4)c3)n2)CC1 ; Cn1nc(C2CCC(=O)NC2=O)c2ccc(C3CCN(CC4CCN(CC(=O)N5CCC(Nc6ncc(Cl)c(-c7cccc(-c8ccc(F)cc8)c7)n6)CC5)CC4)CC3)cc21]

Synthesis of tert-butyl 4-((4-(3-(2,6-dioxopiperidin-3-yl)-1-methyl-1H-indazol-6-yl)piperidin-1-yl)methyl)piperidine-1-carboxylate. To a solution of 3-(1-methyl-6-(piperidin-4-yl)-1H-indazol-3-yl)piperidine-2,6-dione (750 mg, 2.30 mmol, 1 eq) in MeOH (8 mL) was added dropwise tert-butyl 4-formylpiperidine-1-carboxylate (1.96 g, 9.19 mmol, 4 eq) and HOAc (689.93 mg, 11.49 mmol, 657.70 μL, 5 eq) at 25 °C over 1 h. After addition, the mixture was stirred at this temperature for 3 h, and then NaBH₃CN (288.79 mg, 4.60 mmol, 2 eq) was added dropwise at 25 °C. The resulting mixture was stirred at 25 °C for 12 h. LC-MS showed 3-(1-methyl-6-(piperidin-4-yl)-1H-indazol-3-yl)piperidine-2,6-dione was consumed completely and the desired mass was detected. The reaction mixture was quenched by addition H₂O 20 mL, and then diluted with H₂O 10 mL and extracted with EtOAc (10 mL*3). The combined organic layers were washed with sat.aq. NaCl (10 mL), dried over Na₂SO₄, filtered and concentrated under reduced pressure. The residue was purified by prep-TLC (SiO₂, Petroleum ether:Ethyl acetate=0:1, Rf=0.28) to give the title compound (600 mg, crude) as a white solid. LCMS (ES⁺): m/z 524.3 [M+H]⁺.

Synthesis of 3-(1-methyl-6-(1-(piperidin-4-ylmethyl)piperidin-4-yl)-1H-indazol-3-yl)piperidine-2,6-dione. To a solution of tert-butyl 4-((4-(3-(2,6-dioxopiperidin-3-yl)-1-methyl-1H-indazol-6-yl)piperidin-1-yl)methyl)piperidine-1-carboxylate (550 mg, 1.05 mmol, 1.00 eq) in DCM (5 mL) was added TFA (598 mg, 5.25 mmol, 390 μL, 5.00 eq) at 20 °C. The mixture was stirred at 20 °C for 30 min. LC-MS showed tert-butyl 4-((4-(3-(2,6-dioxopiperidin-3-yl)-1-methyl-1H-indazol-6-yl)piperidin-1-yl)methyl)piperidine-1-carboxylate was consumed completely and desired mass was detected. The reaction mixture was filtered and the filtrate concentrated under high vacuum. The reaction mixture was filtered and the filtrate concentrated under high vacuum to give the title compound (600 mg, crude) as a green oil. LCMS (ES⁺): m/z 424.2 [M+H]⁺.

Synthesis of 3-(6-(1-((1-(2-(4-((5-chloro-4-(4′-fluoro-[1,1′-biphenyl]-3-yl)pyrimidin-2-yl)amino)piperidin-1-yl)-2-oxoethyl)piperidin-4-yl)methyl)piperidin-4-yl)-1-methyl-1H-indazol-3-yl)piperidine-2,6-dione (Compound 277). To a solution of 3-(1-methyl-6-(1-(piperidin-4-ylmethyl)piperidin-4-yl)-1H-indazol-3-yl)piperidine-2,6-dione (300 mg, 708 μmol, 1.00 eq) in DMF (3 mL) was added DIEA (274 mg, 2.12 mmol, 370 μL, 3.00 eq) and 2-bromo-1-(4-((5-chloro-4-(4′-fluoro-[1,1′-biphenyl]-3-yl)pyrimidin-2-yl)amino)piperidin-1-yl)ethan-1-one (356 mg, 708 μmol, 1.00 eq) at 20 °C. The mixture was stirred at 20 °C for 30 min. LC-MS showed 3-(1-methyl-6-(1-(piperidin-4-ylmethyl)piperidin-4-yl)-1H-indazol-3-yl)piperidine-2,6-dione was consumed completely and the desired mass was detected. The reaction mixture was filtered and the filtrate concentrated under high vacuum. The residue was purified by prep-HPLC (3_Phenomenex Luna C18 75*30 mm*3 um; mobile phase: [H₂O (0.1% TFA)-ACN]; gradient: 15%-45% B over 8.0 min) to give the title compound (Compound 277) (34.0 mg, 33.7 μmol, 4.76% yield, 95.3% purity, TFA) as a white solid. LCMS (ES⁺): m/z 846.4 [M+H]⁺.

### Example 305. Synthesis of 3-(6-(1-(1-(2-(4-((5-chloro-4-(4′-fluoro-[1,1′-biphenyl]-3-yl)pyrimidin-2-yl)amino)piperidin-1-yl)-2-oxoethyl)-4-hydroxypiperidine-4-carbonyl)piperidin-4-yl)-1-methyl-1H-indazol-3-yl)piperidine-2,6-dione (Compound

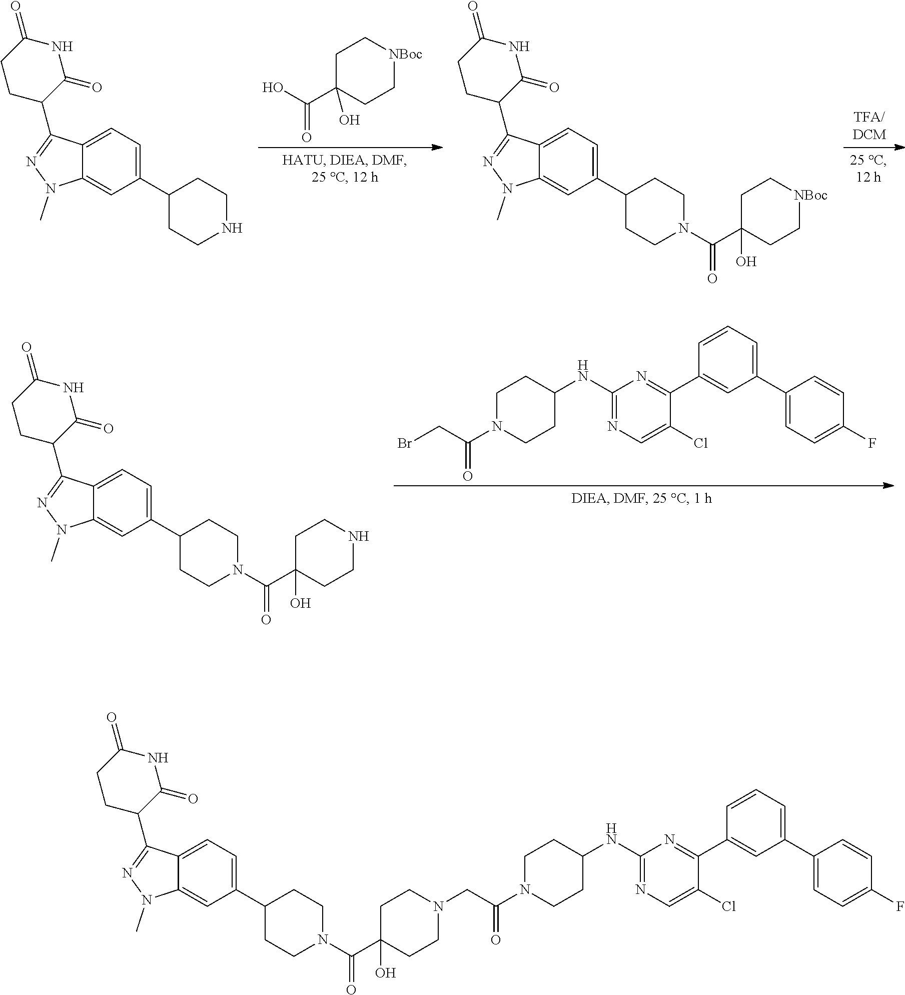 [SMILES: Cn1nc(C2CCC(=O)NC2=O)c2ccc(C3CCNCC3)cc21 ; CC(C)(C)OC(=O)N1CCC(O)(C(=O)O)CC1 ; Cn1nc(C2CCC(=O)NC2=O)c2ccc(C3CCN(C(=O)C4(O)CCN(C(=O)OC(C)(C)C)CC4)CC3)cc21 ; Cn1nc(C2CCC(=O)NC2=O)c2ccc(C3CCN(C(=O)C4(O)CCNCC4)CC3)cc21 ; O=C(CBr)N1CCC(Nc2ncc(Cl)c(-c3cccc(-c4ccc(F)cc4)c3)n2)CC1 ; Cn1nc(C2CCC(=O)NC2=O)c2ccc(C3CCN(C(=O)C4(O)CCN(CC(=O)N5CCC(Nc6ncc(Cl)c(-c7cccc(-c8ccc(F)cc8)c7)n6)CC5)CC4)CC3)cc21]

Synthesis of tert-butyl 4-(4-(3-(2,6-dioxopiperidin-3-yl)-1-methyl-1H-indazol-6-yl)piperidine-1-carbonyl)-4-hydroxypiperidine-1-carboxylate. To a solution of 3-[1-methyl-6-(4-piperidyl) indazol-3-yl]piperidine-2,6-dione (1.00 g, 3.06 mmol, 1.00 eq) in DMF (6 mL) was added DIEA (395 mg, 3.06 mmol, 533 μL, 1.00 eq) until pH~7. To a solution of 1-tert-butoxycarbonyl-4-hydroxy-piperidine-4-carboxylic acid (901 mg, 3.68 mmol, 1.20 eq) in DMF (5 mL) was added DIEA (435 mg, 3.37 mmol, 587 μL, 1.10 eq) until pH~7. Then HATU (3.49 g, 9.19 mmol, 3.00 eq) was added to the mixture of above two solutions at 25 °C. The mixture was stirred at 25 °C for 12 h. LC-MS showed the desired mass was detected. The reaction diluted with H₂O 20 mL and combined organic layers were washed with EtOAc (30 mL*3), dried over Na₂SO₄, filtered and concentrated under reduced pressure. The residue was purified by column chromatography (SiO₂, Petroleum ether:Ethyl acetate=1:0 to 0:1) to give the title compound (0.80 g, crude) as a yellow solid. LCMS (ES⁺): m/z 554.4 [M+H]⁺.

Synthesis of 3-(6-(1-(4-hydroxypiperidine-4-carbonyl)piperidin-4-yl)-1-methyl-1H-indazol-3-yl)piperidine-2,6-dione. A solution of tert-butyl 4-(4-(3-(2,6-dioxopiperidin-3-yl)-1-methyl-1H-indazol-6-yl)piperidine-1-carbonyl)-4-hydroxypiperidine-1-carboxylate (800 mg, 1.44 mmol, 1.00 eq) in DCM (10 mL) was added TFA (3.07 g, 26.9 mmol, 2 mL, 18.6 eq) at 25 °C. The mixture was stirred at 25 °C for 12 h. LC-MS showed the desired mass was detected. The reaction mixture was concentrated under reduced pressure. The residue was purified by prep-HPLC (column: Phenomenex luna C18 100*40 mm*5 um; mobile phase: [H₂O (0.2% FA)-ACN]; gradient: 1%-30% B over 8.0 min) to give the title compound (30.0 mg, 48.2 μmol, 2.19% yield, 73.0% purity) as a yellow solid. LCMS (ES⁺): m/z 454.2 [M+H]⁺.

Synthesis of 3-(6-(1-(1-(2-(4-((5-chloro-4-(4′-fluoro-[1,1′-biphenyl]-3-yl)pyrimidin-2-yl)amino)piperidin-1-yl)-2-oxoethyl)-4-hydroxypiperidine-4-carbonyl)piperidin-4-yl)-1-methyl-1H-indazol-3-yl)piperidine-2,6-dione (Compound 278). To a solution of 3-(6-(1-(4-hydroxypiperidine-4-carbonyl)piperidin-4-yl)-1-methyl-1H-indazol-3-yl)piperidine-2,6-dione (30.0 mg, 66.1 μmol, 1.00 eq) in DMF (1 mL) was added DIEA (8.55 mg, 66.1 μmol, 11.5 μL, 1.00 eq) until pH-8 at 25 °C. The mixture was stirred at 25 °C for 0.5 h. Then 2-bromo-1-[4-[[5-chloro-4-[3-(4-fluorophenyl)phenyl]pyrimidin-2-yl]amino]-1-piperidyl]ethanone (30 mg, 59.6 μmol, 0.900 eq) was added into the solution, The mixture was stirred at 25 °C for 0.5 h. LC-MS showed the desired mass was detected. The reaction mixture was concentrated under reduced pressure. The residue was purified by prep-HPLC (column: 3_Phenomenex Luna C18 75*30 mm*3 um; mobile phase: [H₂O (0.1% TFA)-ACN]; gradient: 35%-65% B over 8.0 min) to give the title compound (Compound 278) (10.0 mg, 10.0 μmol, 15.2% yield, 99.5% purity, TFA) as a light orange solid. LCMS (ES⁺): m/z 876.3 [M+H]⁺.

### Example 306. Synthesis of 3-(5-(1′-(2-(4-((5-chloro-4-(4′-fluoro-[1,1′-biphenyl]-3-yl)pyrimidin-2-yl)amino)piperidin-1-yl)-2-oxoethyl)-[1,3′-bipiperidin]-4-yl)-3-methyl-2-oxo-2,3-dihydro-1H-benzo[d]imidazol-1-yl)piperidine-2,6-dione (Compound 275)

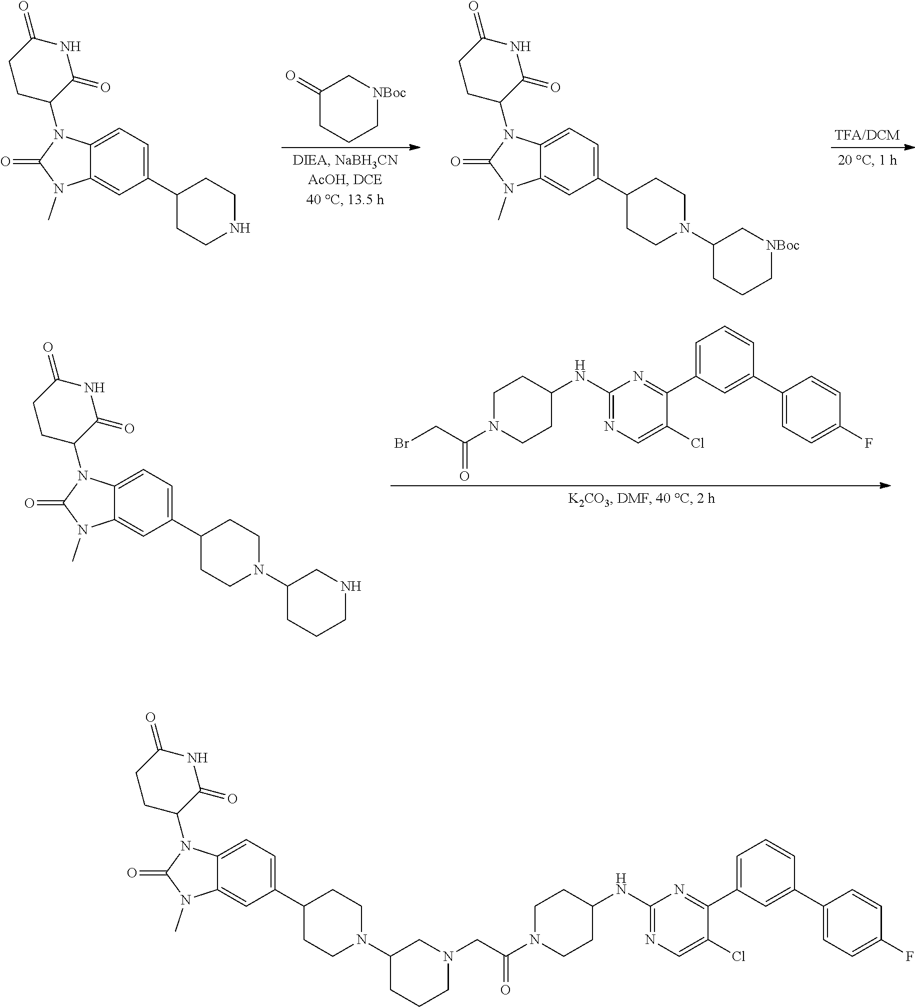

Synthesis of tert-butyl 4-(1-(2,6-dioxopiperidin-3-yl)-3-methyl-2-oxo-2,3-dihydro-1H-benzo[d]imidazol-5-yl)-[1,3′-bipiperidine]-1′-carboxylate. To a solution of 3-(3-methyl-2-oxo-5-(piperidin-4-yl)-2,3-dihydro-1H-benzo[d]imidazol-1-yl)piperidine-2,6-dione (500 mg, 1.10 mmol, 1.00 eq, TFA) in DCE (5 mL) was added DIEA to adjust pH to 8 at 20 °C. The mixture was stirred at 20 °C for 1 h. Then to the reaction mixture were added tert-butyl 3-oxopiperidine-1-carboxylate (873 mg, 4.38 mmol, 4.00 eq) and AcOH (65.8 mg, 1.10 mmol, 62.7 μL, 1.00 eq) at 20 °C. The mixture was stirred at 40 °C for 0.5 h. Then to the reaction mixture was added NaBH₃CN (275 mg, 4.38 mmol, 4.00 eq) at 20 °C. The resulting mixture was stirred at 40 °C for 12 h. LCMS showed 3-(3-methyl-2-oxo-5-(piperidin-4-yl)-2,3-dihydro-1H-benzo[d]imidazol-1-yl)piperidine-2,6-dione was consumed and the desired mass was detected. To the mixture was added H₂O (10 mL) and the mixture was extracted with EtOAc (10 mL*3). The combined organic layers were washed with brine (10 mL*3), dried over Na₂SO₄, filtered and the filtrate was concentrated under reduced pressure to give a residue. The residue was purified by prep-TLC (SiO₂, Dichloromethane/Methanol=5/1) to give the title compound (210 mg, 400 μmol, 36.5% yield) as a yellow solid. LCMS (ES⁺): m/z 526.1 [M+H]⁺.

Synthesis of 3-(5-([1,3′-bipiperidin]-4-yl)-3-methyl-2-oxo-2,3-dihydro-1H-benzo[d]imidazol-1-yl)piperidine-2,6-dione. A solution of tert-butyl 4-(1-(2,6-dioxopiperidin-3-yl)-3-methyl-2-oxo-2,3-dihydro-1H-benzo[d]imidazol-5-yl)-[1,3′-bipiperidine]-1′-carboxylate (180 mg, 342 μmol, 1.00 eq) in DCM (3 mL) was added TFA (0.6 mL) at 20 °C. The mixture was stirred at 20 °C for 1 h. LCMS showed tert-butyl 4-(1-(2,6-dioxopiperidin-3-yl)-3-methyl-2-oxo-2,3-dihydro-1H-benzo[d]imidazol-5-yl)-[1,3′-bipiperidine]-1′-carboxylate was consumed and the desired mass was detected. The reaction mixture was concentrated under reduced pressure to give the title compound (180 mg, crude, TFA) as a yellow oil. LCMS (ES⁺): m/z 426.1 [M+H]⁺.

Synthesis of 3-(5-(1′-(2-(4-((5-chloro-4-(4′-fluoro-[1,1′-biphenyl]-3-yl)pyrimidin-2-yl)amino)piperidin-1-yl)-2-oxoethyl)-[1,3′-bipiperidin]-4-yl)-3-methyl-2-oxo-2,3-dihydro-1H-benzo[d]imidazol-1-yl)piperidine-2,6-dione (Compound 275). To a solution of 3-(5-([1,3′-bipiperidin]-4-yl)-3-methyl-2-oxo-2,3-dihydro-1H-benzo[d]imidazol-1-yl)piperidine-2,6-dione (180 mg, 334 μmol, 1.00 eq, TFA) in DMF (3 mL) were added 2-bromo-1-(4-((5-chloro-4-(4′-fluoro-[1,1′-biphenyl]-3-yl)pyrimidin-2-yl)amino)piperidin-1-yl)ethan-1-one (168 mg, 334 μmol, 1.00 eq) and K₂CO₃ (138 mg, 1.00 mmol, 3.00 eq) at 20 °C. The mixture was stirred at 40 °C for 2 h. LCMS showed 3-(5-([1,3′-bipiperidin]-4-yl)-3-methyl-2-oxo-2,3-dihydro-1H-benzo[d]imidazol-1-yl)piperidine-2,6-dione was consumed and the desired mass was detected. The mixture was filtered and the filtrate was concentrated under reduced pressure to give a residue. The residue was purified by prep-HPLC (column: Phenomenex Luna C18 75*30 mm*3 um; mobile phase: [H₂O (0.1% TFA)-ACN]; gradient: 10%-50% B over 8.0 min) to give the title compound (Compound 275) (38.5 mg, 45.4 umol, 13.6% yield, 100% purity) as a yellow solid. LCMS (ES⁺): m/z 848.4 [M+H]⁺

### Example 307. Synthesis of 3-(5-(1-((1-(2-(4-((5-chloro-4-(4′-fluoro-[1,1′-biphenyl]-3-yl)pyrimidin-2-yl)amino)piperidin-1-yl)-2-oxoethyl)piperidin-4-yl)methyl)piperidin-4-yl)-3-methyl-2-oxo-2,3-dihydro-1H-benzo[d]imidazol-1-yl)piperidine-2,6-dione (Compound 287)

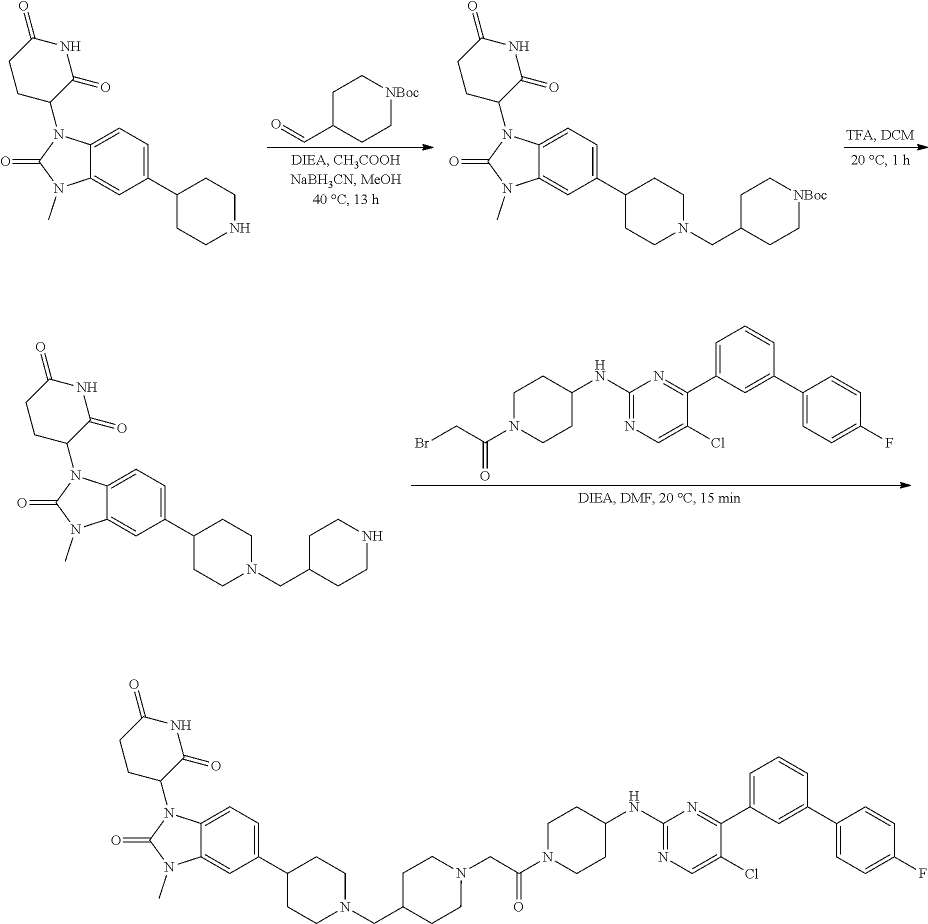 [SMILES: Cn1c(=O)n(C2CCC(=O)NC2=O)c2ccc(C3CCNCC3)cc21 ; CC(C)(C)OC(=O)N1CCC(C=O)CC1 ; Cn1c(=O)n(C2CCC(=O)NC2=O)c2ccc(C3CCN(CC4CCN(C(=O)OC(C)(C)C)CC4)CC3)cc21 ; Cn1c(=O)n(C2CCC(=O)NC2=O)c2ccc(C3CCN(CC4CCNCC4)CC3)cc21 ; O=C(CBr)N1CCC(Nc2ncc(Cl)c(-c3cccc(-c4ccc(F)cc4)c3)n2)CC1 ; Cn1c(=O)n(C2CCC(=O)NC2=O)c2ccc(C3CCN(CC4CCN(CC(=O)N5CCC(Nc6ncc(Cl)c(-c7cccc(-c8ccc(F)cc8)c7)n6)CC5)CC4)CC3)cc21]

Synthesis of tert-butyl 4-((4-(1-(2,6-dioxopiperidin-3-yl)-3-methyl-2-oxo-2,3-dihydro-1H-benzo[d]imidazol-5-yl)piperidin-1-yl)methyl)piperidine-1-carboxylate. To a solution of 3-[3-methyl-2-oxo-5-(4-piperidyl)benzimidazol-1-yl]piperidine-2,6-dione (6.70 g, 14.7 mmol, 1.00 eq, TFA) in MeOH (100 mL) was added DIEA at 20 °C. DIEA was added dropwise until the pH had reached 8. The mixture was stirred at 20 °C for 0.5 h. CH₃COOH (4.41 g, 73.4 mmol, 4.20 mL, 5.00 eq) and tert-butyl 4-formylpiperidine-1-carboxylate (3.76 g, 17.6 mmol, 1.20 eq) were added dropwise until the pH had reached 5 at 20 °C. The mixture was stirred at 20 °C for 0.5 h. Then NaBH₃CN (1.84 g, 29.4 mmol, 2.00 eq) was added to the mixture at 20 °C. The mixture was stirred at 40 °C for 12 h. LCMS showed 3-[3-methyl-2-oxo-5-(4-piperidyl)benzimidazol-1-yl]piperidine-2,6-dione was consumed completely and the desired mass was detected. The reaction mixture was concentrated under reduced pressure to give a residue. The residue was purified by column chromatography (SiO₂, Dichloromethane:Methanol=1:0 to 10:1) to give the title compound (6.15 g, 11.4 mmol, 77.6% yield) as a colorless oil. LCMS (ES⁺): m/z 540.6 [M+H]⁺.

Synthesis of 3-(3-methyl-2-oxo-5-(1-(piperidin-4-ylmethyl)piperidin-4-yl)-2,3-dihydro-1H-benzo[d]imidazol-1-yl)piperidine-2,6-dione. To a solution of tert-butyl 4-((4-(1-(2,6-dioxopiperidin-3-yl)-3-methyl-2-oxo-2,3-dihydro-1H-benzo[d]imidazol-5-yl)piperidin-1-yl)methyl)piperidine-1-carboxylate (300 mg, 556 μmol, 1.00 eq) in DCM (6 mL) was added TFA (2 mL) at 20 °C. The mixture was stirred at 20 °C for 1 h. LCMS showed tert-butyl 4-((4-(1-(2,6-dioxopiperidin-3-yl)-3-methyl-2-oxo-2,3-dihydro-1H-benzo[d]imidazol-5-yl)piperidin-1-yl)methyl)piperidine-1-carboxylate was consumed completely and the desired mass was detected. The reaction mixture was concentrated under reduced pressure to remove solvent to give the title compound (300 mg, crude, TFA) as a yellow oil. LCMS (ES⁺): m/z 440.4 [M+H]⁺.

Synthesis of 3-(5-(1-((1-(2-(4-((5-chloro-4-(4′-fluoro-[1,1′-biphenyl]-3-yl)pyrimidin-2-yl)amino)piperidin-1-yl)-2-oxoethyl)piperidin-4-yl)methyl)piperidin-4-yl)-3-methyl-2-oxo-2,3-dihydro-1H-benzo[d]imidazol-1-yl)piperidine-2,6-dione (Compound 287). To a solution of 3-(3-methyl-2-oxo-5-(1-(piperidin-4-ylmethyl)piperidin-4-yl)-2,3-dihydro-1H-benzo[d]imidazol-1-yl)piperidine-2,6-dione (170 mg, 357 μmol, 1.00 eq, HCl) in DMF (2 mL) were added DIEA (139 mg, 1.07 mmol, 189 μL, 3.00 eq) and 2-bromo-1-(4-((5-chloro-4-(4′-fluoro-[1,1′-biphenyl]-3-yl)pyrimidin-2-yl)amino)piperidin-1-yl)ethan-1-one (180 mg, 357 μmol, 1.00 eq) at 0 °C. The mixture was stirred at 20 °C for 15 min. LCMS showed that 2-bromo-1-(4-((5-chloro-4-(4′-fluoro-[1,1′-biphenyl]-3-yl)pyrimidin-2-yl)amino)piperidin-1-yl)ethan-1-one was consumed completely and the desired mass was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 15%-55% B over 8.0 min) to give the title compound (Compound 287) (34.7 mg, 37.3 μmol, 10.4% yield, 97.6% purity, FA) as a white solid. LCMS (ES⁺): m/z 862.1 [M+H]⁺

### Example 308. Synthesis of 3-(4-((1′-(2-(4-((5-chloro-4-(4′-fluoro-[1,1′-biphenyl]-3-yl)pyrimidin-2-yl)amino)piperidin-1-yl)-2-oxoethyl)-[1,3′-bipiperidin]-4-yl)oxy)-1-oxoisoindolin-2-yl)piperidine-2,6-dione (Compound 296)

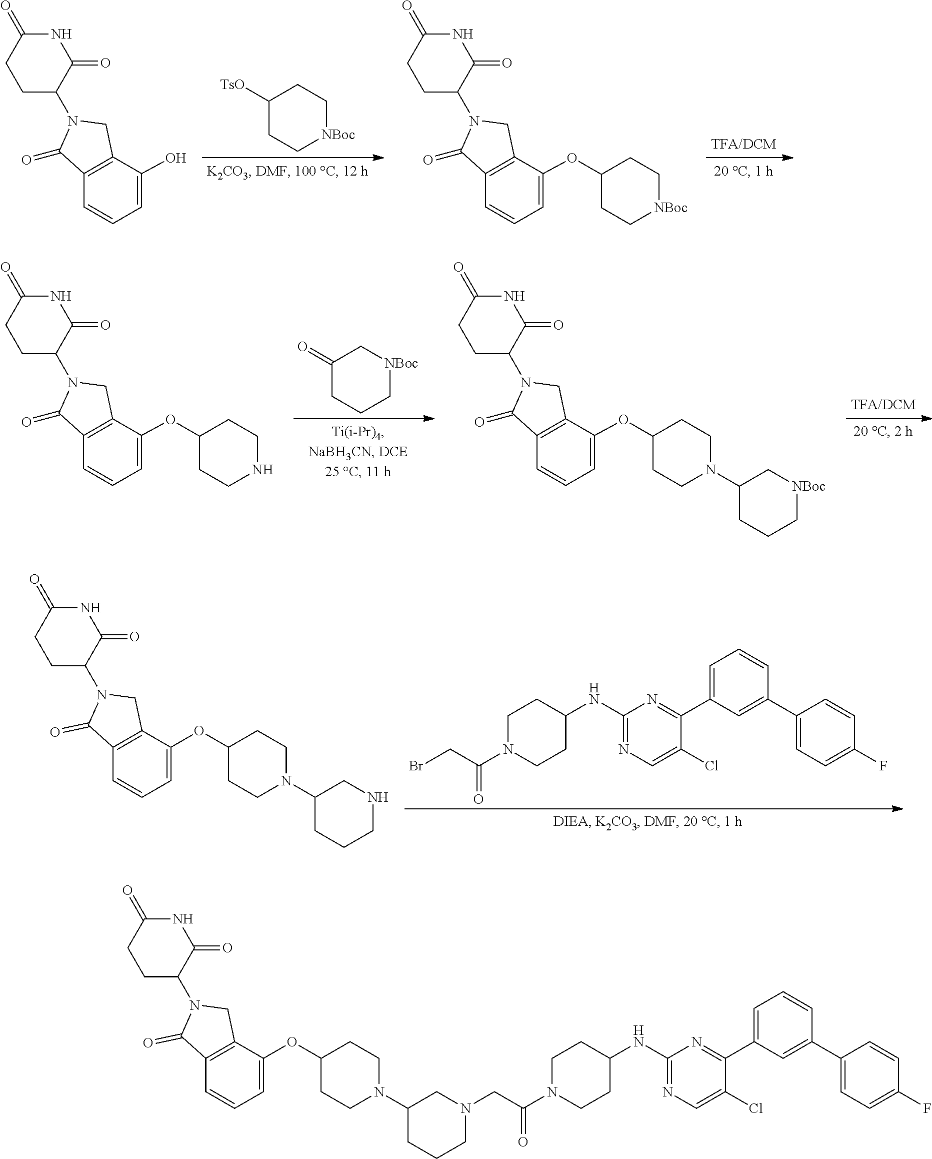

Synthesis of tert-butyl 4-[2-(2,6-dioxo-3-piperidyl)-1-oxo-isoindolin-4-yl]oxypiperidine-1-carboxylate. A mixture of 3-(4-hydroxy-1-oxo-isoindolin-2-yl)piperidine-2,6-dione (5.00 g, 19.2 mmol, 1.00 eq), tert-butyl 4-(p-tolylsulfonyloxy)piperidine-1-carboxylate (8.19 g, 23.0 mmol, 1.20 eq), K₂CO₃ (7.97 g, 57.6 mmol, 3.00 eq) in DMF (50 mL) was degassed and purged with N₂ at 20 °C for 3 times, and then the mixture was stirred at 100 °C for 12 h under N₂ atmosphere. LC-MS showed the desired mass was detected. The reaction mixture was quenched by addition H₂O 30 mL, and then diluted with H₂O 10 mL and extracted with EtOAc (50 mL*3). The combined organic layers were washed with sat.aq. NaCl (10 mL), dried over Na₂SO₄, filtered and concentrated under reduced pressure. The residue was purified by column chromatography (SiO₂, Petroleum ether:Ethyl acetate=1:0 to 0:1) and TLC (Petroleum ether:Ethyl acetate=0:1, Rf=0.56) to give the title compound (5.50 g, 8.10 mmol, 42.1% yield, 65.2% purity) as a white solid. LCMS (ES⁺): m/z 388.1 [M−56+H]⁺.

Synthesis of tert-butyl 4-((2-(2,6-dioxopiperidin-3-yl)-1-oxoisoindolin-4-yl)oxy)-[1,3′-bipiperidine]-1′-carboxylate. To a solution of tert-butyl 4-[2-(2,6-dioxo-3-piperidyl)-1-oxo-isoindolin-4-yl]oxypiperidine-1-carboxylate (4.00 g, 9.02 mmol, 1.00 eq) in DCM (10 mL) was added TFA (10.2 g, 90.1 mmol, 6.70 mL, 10.0 eq) at 20 °C. The mixture was stirred at 20 °C for 1 h. LC-MS showed the desired mass was detected. The reaction mixture was filtered and the filtrate concentrated under high vacuum. The reaction mixture was filtered and the filtrate concentrated under high vacuum to give the title compound (3.80 g, crude, TFA) as a yellow oil. LCMS (ES⁺): m/z 344.2 [M+H]⁺.

Synthesis of tert-butyl 4-((2-(2,6-dioxopiperidin-3-yl)-1-oxoisoindolin-4-yl)oxy)-[1,3′-bipiperidine]-1′-carboxylate. To a solution of 3-(1-oxo-4-(piperidin-4-yloxy)isoindolin-2-yl)piperidine-2,6-dione (1.50 g, 4.37 mmol, TFA, 1.00 eq) in DCE (40 mL) was added dropwise tetraethoxytitanium (4.98 g, 21.8 mmol, 4.53 mL, 5.00 eq) and tert-butyl 3-oxopiperidine-1-carboxylate (4.35 g, 21.8 mmol, 5.00 eq) at 25 °C. After addition, the mixture was stirred at this temperature for 1 h, and then NaBH₃CN (1.37 g, 21.8 mmol, 5.00 eq) was added at 25 °C. The resulting mixture was stirred at 25 °C for 10 h. LC-MS showed the desired mass was detected. The reaction mixture was filtered and the filtrate was diluted with H₂O 50 mL and extracted with EtOAc 40 mL*3. The combined organic layers were dried over Na₂SO₄, filtered and concentrated under reduced pressure. The residue was purified by column chromatography (SiO₂, PE:EtOAc=1:0 to 0:1) to give the title compound (2.5 g, crude) as a yellow oil. LCMS (ES⁺): m/z 527.3 [M+H]⁺.

Synthesis of 3-(4-([1,3′-bipiperidin]-4-yloxy)-1-oxoisoindolin-2-yl)piperidine-2,6-dione. To a solution of tert-butyl 4-((2-(2,6-dioxopiperidin-3-yl)-1-oxoisoindolin-4-yl)oxy)-[1,3′-bipiperidine]-1′-carboxylate (850 mg, 1.61 mmol, 1.00 eq) in DCM (4 mL) was added TFA (2 mL) at 20 °C. The mixture was stirred at 20 °C for 2 h. LC-MS showed the desired mass was detected. The reaction mixture was concentrated under reduced pressure to give the title compound (1.5 g, crude, TFA) as a white oil. LCMS (ES⁺): m/z 427.3 [M+H]⁺.

Synthesis of 3-(4-((1′-(2-(4-((5-chloro-4-(4′-fluoro-[1,1′-biphenyl]-3-yl)pyrimidin-2-yl)amino)piperidin-1-yl)-2-oxoethyl)-[1,3′-bipiperidin]-4-yl)oxy)-1-oxoisoindolin-2-yl)piperidine-2,6-dione (Compound 296). To a solution of 3-(4-([1,3′-bipiperidin]-4-yloxy)-1-oxoisoindolin-2-yl)piperidine-2,6-dione (150 mg, 351 ji mol, 1.00 eq) and 2-bromo-1-[4-[[5-chloro-4-[3-(4-fluorophenyl)phenyl]pyrimidin-2-yl]amino]-1-piperidyl]ethanone (177 mg, 351 μmol, 1.00 eq) in DMF (5 mL) was added DIEA (136 mg, 1.06 mmol, 183 μL, 3.00 eq) and K₂CO₃ (145 mg, 1.06 mmol, 3.00 eq) at 20 °C. The mixture was stirred at 20 °C for 1 h. LC-MS showed the desired mass was detected. The reaction mixture was filtered and concentrated under reduced pressure. The residue was purified by prep-HPLC (column: 3_Phenomenex Luna C18 75*30 mm*3 um; mobile phase: [H₂O (0.1% TFA)-ACN]; gradient: 25%-55% B over 8.0 min) to give the title compound (Compound 296). (14.2 mg, 15.68 μmol, 4.46% yield, 93.8% purity) as a yellow solid. LCMS (ES⁺): m/z 849.3 [M+H]⁺.

### Example 309. Synthesis of cis-3-(4-(4-(2-(4-(3-((5-chloro-4-(4′-fluoro-[1,1′-biphenyl]-3-yl)pyrimidin-2-yl)amino)cyclohexane-1-carbonyl)piperazin-1-yl)-2-oxoethyl)-4-hydroxypiperidin-1-yl)-3-fluorophenoxy)piperidine-2,6-dione (Compound 292)

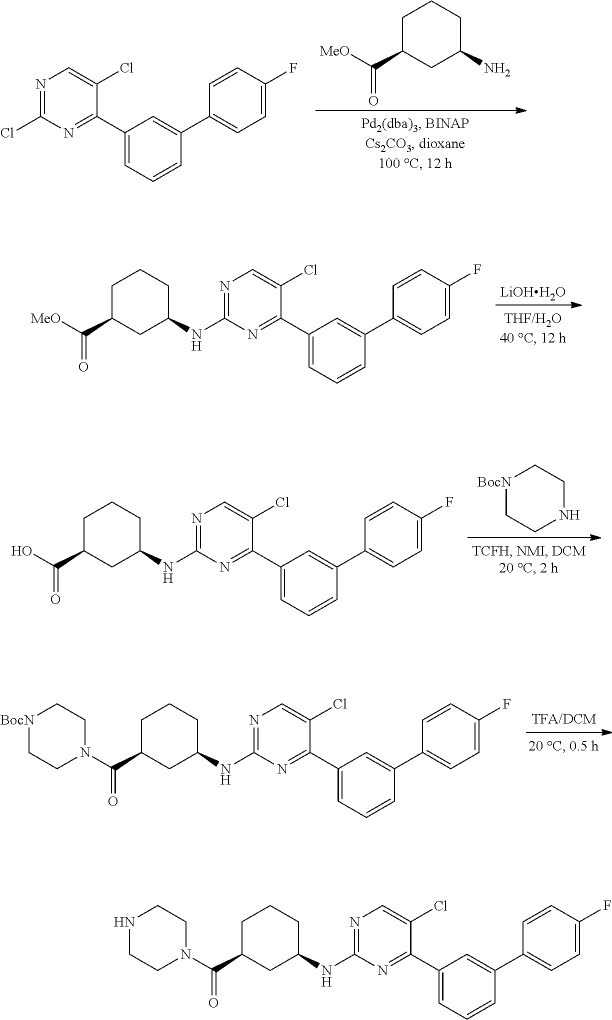

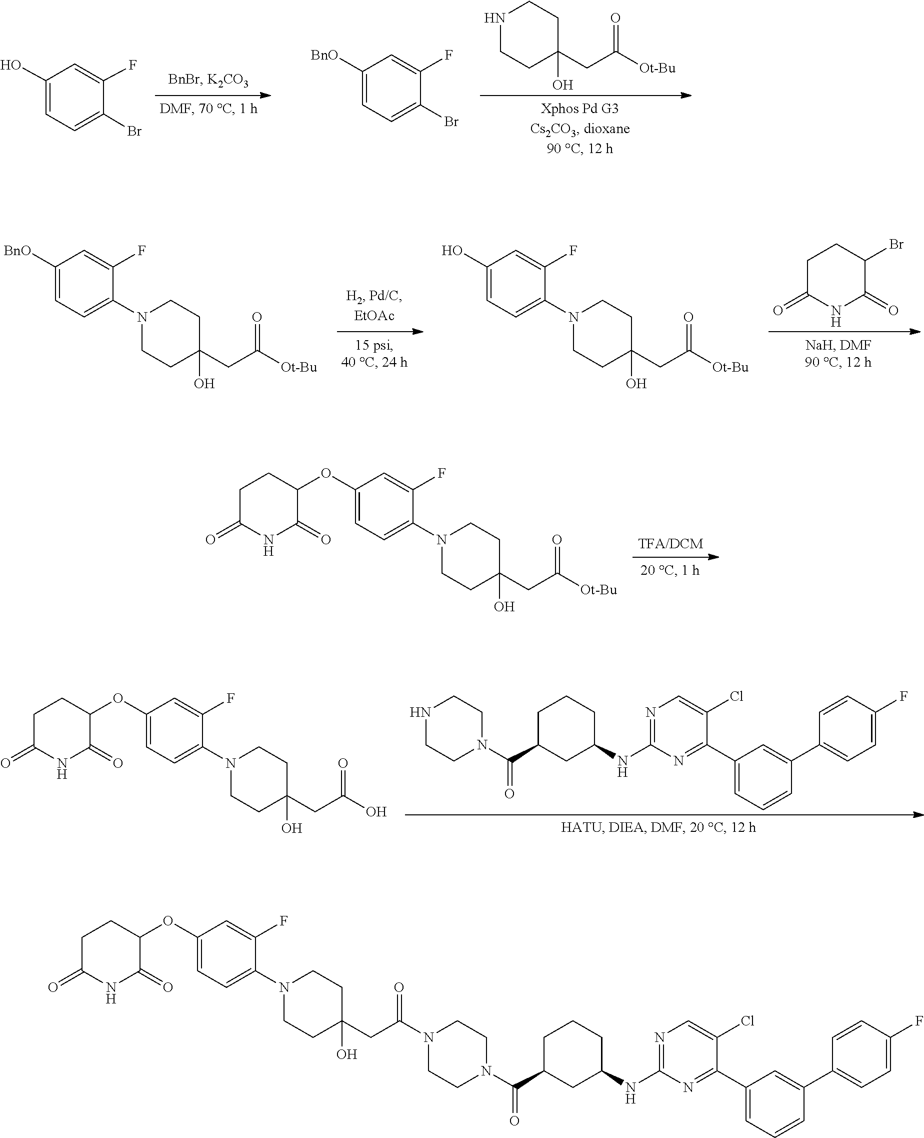

Synthesis of cis-methyl 3-((5-chloro-4-(4′-fluoro-[1,1′-biphenyl]-3-yl)pyrimidin-2-yl)amino)cyclohexane-1-carboxylate. To a solution of 2,5-dichloro-4-(4′-fluoro-[1,1′-biphenyl]-3-yl)pyrimidine (6.5 g, 20.37 mmol, 1.20 eq) in dioxane (100 mL) were added cis-methyl 3-aminocyclohexane-1-carboxylate (3.29 g, 16.97 mmol, 1.00 eq, HCl), Pd₂(dba)₃ (1.55 g, 1.70 mmol, 0.100 eq), Cs₂CO₃ (16.6 g, 50.9 mmol, 3.00 eq) and BINAP (1.06 g, 1.70 mmol, 0.100 eq) at 20 °C under N₂. The mixture was stirred at 100 °C for 12 h under N₂. LC-MS showed 2,5-dichloro-4-(4′-fluoro-[1,1′-biphenyl]-3-yl)pyrimidine was consumed completely and the desired mass was detected. Water (80 mL) was added to the mixture at 25 °C. Then the mixture was extracted with EtOAc (50 mL*I 3). The organic layers were dried over anhydrous sodium sulfate, filtered and the filtrate was concentrated under reduced pressure to give a residue. The residue was purified by column chromatography (SiO₂, Petroleum ether/Ethyl acetate=5/1 to 3/1) to give the title compound (4.1 g, 9.32 mmol, 54.9% yield) as a white solid. LCMS (ES+): m/z 440.1 [M+H]⁺.

Synthesis of cis-3-((5-chloro-4-(4′-fluoro-[1,1′-biphenyl]-3-yl)pyrimidin-2-yl)amino)cyclohexane-1-carboxylic acid. To a solution of cis-methyl 3-((5-chloro-4-(4′-fluoro-[1,1′-biphenyl]-3-yl)pyrimidin-2-yl)amino)cyclohexane-1-carboxylate (4.10 g, 9.32 mmol, 1.00 eq) in THE (45 mL) and H₂O (15 mL) was added LiOH·H₂O (1.96 g, 46.6 mmol, 5.00 eq) at 20 °C. The mixture was stirred at 40 °C for 12 h. LCMS showed cis-methyl 3-((5-chloro-4-(4′-fluoro-[1,1′-biphenyl]-3-yl)pyrimidin-2-yl)amino)cyclohexane-1-carboxylate was consumed completely and the desired mass was detected. The reaction was concentrated under reduced pressure to remove THF. HCl (1M) was added to the remaining aqueous phase to adjust pH to 2-3. Then it was extracted with EtOAc (30 mL*3). The organic layers were dried over anhydrous sodium sulfate, filtered and the filtrate was concentrated under reduced pressure to give to give the title compound (3.9 g, crude) as a yellow solid. LCMS (ES+): m/z 426.1 [M+H]⁺.

Synthesis of cis-tert-butyl 4-(3-((5-chloro-4-(4′-fluoro-[1,1′-biphenyl]-3-yl)pyrimidin-2-yl)amino)cyclohexane-1-carbonyl)piperazine-1-carboxylate. To a solution of tert-butyl piperazine-1-carboxylate (219 mg, 1.17 mmol, 1.00 eq) in DCM (10 mL) were added cis-3-((5-chloro-4-(4′-fluoro-[1,1′-biphenyl]-3-yl)pyrimidin-2-yl)amino)cyclohexane-1-carboxylic acid (500 mg, 1.17 mmol, 1.00 eq), TCFH (494 mg, 1.76 mmol, 1.50 eq) and NMI (482 mg, 5.87 mmol, 468 μL, 5.00 eq) at 20 °C. The mixture was stirred at 20 °C for 2 h. LCMS showed cis-3-((5-chloro-4-(4′-fluoro-[1,1-biphenyl]-3-yl)pyrimidin-2-yl)amino)cyclohexane-1-carboxylic acid was consumed completely and the desired mass was detected. Water (10 mL) was added to the mixture at 20 °C. Then the mixture was extracted with DCM (5 mL*3). The organic layers were dried over anhydrous sodium sulfate, filtered and the filtrate was concentrated under reduced pressure to give a residue. The residue was purified by prep-TLC (SiO₂, Ethyl acetate/Petroleum ether=2/1) to give the title compound (550 mg, 926 μmol, 78.9% yield) as a white solid. LCMS (ES+): m/z 594.4 [M+H]⁺.

Synthesis of cis-(3-((5-chloro-4-(4′-fluoro-[1,1′-biphenyl]-3-yl)pyrimidin-2-yl)amino)cyclohexyl)(piperazin-1-yl)methanone. A solution of cis-tert-butyl 4-(3-((5-chloro-4-(4′-fluoro-[1,1′-biphenyl]-3-yl)pyrimidin-2-yl)amino)cyclohexane-1-carbonyl)piperazine-1-carboxylate (130 mg, 219 μmol, 1.00 eq) in DCM (2.5 mL) and TFA (0.5 mL) was stirred at 20 °C for 0.5 h. LCMS showed that cis-tert-butyl 4-(3-((5-chloro-4-(4′-fluoro-[1,1′-biphenyl]-3-yl)pyrimidin-2-yl)amino)cyclohexane-1-carbonyl)piperazine-1-carboxylate was consumed completely and the desired mass was detected. The mixture was concentrated under reduced pressure to give the title compound (130 mg, crude, TFA) as a yellow oil. LCMS (ES+): m/z 494.3 [M+H]⁺.

Synthesis of 4-(benzyloxy)-1-bromo-2-fluorobenzene. To a solution of 4-bromo-3-fluoro-phenol (20.0 g, 105 mmol, 1.00 eq) in DMF (300 mL) were added bromomethylbenzene (19.3 g, 113 mmol, 13.4 mL, 1.08 eq) and K₂CO₃ (28.94 g, 209.42 mmol, 2.00 eq) at 25 °C. The mixture was stirred at 70 °C for 1 h. TLC (SiO₂, Petroleum ether/Ethyl acetate=10/1) indicated that the 4-bromo-3-fluoro-phenol was consumed completely and one new spot formed. H₂O (300 mL) was added to the mixture and the mixture was extracted with EtOAc (200 mL*3), the combined organic layers were washed with brine (200 mL*3), dried over Na₂SO₄, filtered and the filtrate was concentrated under reduced pressure to give a residue. The residue was purified by column chromatography (SiO₂, Petroleum ether/Ethyl acetate=1/0 to 10/1) to give the title compound (18.2 g, 64.85 mmol, 61.9% yield) as a white solid. It was confirmed by ¹H HNMR. ¹H NMR (400 MHz, CDCl3) δ (ppm)=7.44-7.37 (m, 6H), 6.80-6.77 (m, 1H), 6.71-6.69 (m, 1H), 5.04 (s, 2H)

Synthesis of tert-butyl 2-(1-(4-(benzyloxy)-2-fluorophenyl)-4-hydroxypiperidin-4-yl)acetate. To a solution of 4-(benzyloxy)-1-bromo-2-fluorobenzene (2.00 g, 7.11 mmol, 1.00 eq) in dioxane (50 mL) were added tert-butyl 2-(4-hydroxypiperidin-4-yl)acetate (4.59 g, 21.3 mmol, 3.00 eq), XPhos Pd G3 (602 mg, 711 μmol, 0.100 eq) and Cs₂CO₃ (6.95 g, 21.3 mmol, 3.00 eq) at 20 °C under N₂ atmosphere. The mixture was stirred at 90 °C for 12 h under N₂. LCMS showed 4-(benzyloxy)-1-bromo-2-fluorobenzene was consumed and the desired mass was detected. H₂O (80 mL) was added to the mixture and the mixture was extracted with EtOAc (50 mL*3), the combined organic layers were dried over Na₂SO₄, filtered and the filtrate was concentrated under reduced pressure to give a residue. The residue was purified by MPLC (SiO₂, PE/EtOAc=1/0 to 1/1) to give the title compound (700 mg, 1.68 mmol, 23.7% yield) as a yellow oil. LCMS (ES+): m/z 416.2 [M+H]⁺.

Synthesis of benzyl tert-butyl 2-(1-(2-fluoro-4-hydroxyphenyl)-4-hydroxypiperidin-4-yl)acetate. To a stirred solution of tert-butyl 2-(1-(4-(benzyloxy)-2-fluorophenyl)-4-hydroxypiperidin-4-yl)acetate (700 mg, 1.68 mmol, 1.00 eq) in EtOAc (15 mL) was added 10% Pd/C (700 mg, 50% purity) at 20 °C. The mixture was stirred at 40 °C for 12 h under H₂ (15 psi). LCMS showed that the tert-butyl 2-(1-(4-(benzyloxy)-2-fluorophenyl)-4-hydroxypiperidin-4-yl)acetate remained and the desired mass was detected. Then 10% Pd/C (800 mg, 50% purity) was added to the mixture. The mixture was stirred at 40 °C for another 12 h under H₂ (15 psi). LCMS showed that tert-butyl 2-(1-(4-(benzyloxy)-2-fluorophenyl)-4-hydroxypiperidin-4-yl)acetate was consumed completely and the desired mass was detected. The mixture was filtered and the filtrate was concentrated under reduced pressure to give a residue. The residue was purified by prep-TLC (SiO₂, PE/EtOAc=1/1) to give the title compound (260 mg, 799 μmol, 47.4% yield) as a yellow solid. LCMS (ES⁺): m/z 326.2 [M+H]⁺.

Synthesis of tert-butyl 2-(1-(4-((2,6-dioxopiperidin-3-yl)oxy)-2-fluorophenyl)-4-hydroxypiperidin-4-yl)acetate. To a stirred solution of tert-butyl 2-(1-(2-fluoro-4-hydroxyphenyl)-4-hydroxypiperidin-4-yl)acetate (260 mg, 799 μmol, 1.00 eq) in DMF (5 mL) was added NaH (160 mg, 4.00 mmol, 60% purity, 5.00 eq) at 0 °C and the mixture was stirred at 0 °C for 0.5 h. Then to the mixture was added 3-bromopiperidine-2,6-dione (921 mg, 4.79 mmol, 6.00 eq) at 20 °C and the resulting mixture was stirred at 90 °C for 11.5 h. LCMS showed that tert-butyl 2-(1-(2-fluoro-4-hydroxyphenyl)-4-hydroxypiperidin-4-yl)acetate was consumed and the desired mass was detected. The reaction mixture was quenched by ice water 10 mL at 0 °C and the mixture was extracted with EtOAc (10 mL*3). The combined organic layers were washed with brine (10 mL*3), dried over Na₂SO₄, filtered and the filtrate was concentrated under reduced pressure to give a residue. The residue was purified by prep-TLC (SiO₂, PE/EtOAc=1/3) to give the title compound (180 mg, 412 μmol, 51.6% yield) as a yellow oil. LCMS (ES⁺): m/z 437.2 [M+H]⁺.

Synthesis of 2-(1-(4-((2,6-dioxopiperidin-3-yl)oxy)-2-fluorophenyl)-4-hydroxypiperidin-4-yl)acetic acid. A solution of tert-butyl 2-(1-(4-((2,6-dioxopiperidin-3-yl)oxy)-2-fluorophenyl)-4-hydroxypiperidin-4-yl)acetate (180 mg, 412 μmol, 1.00 eq) in TFA (0.5 mL) and DCM (2.5 mL) was stirred at 20 °C for 1 h. LCMS showed tert-butyl 2-(1-(4-((2,6-dioxopiperidin-3-yl)oxy)-2-fluorophenyl)-4-hydroxypiperidin-4-yl)acetate was consumed completely. The reaction mixture was concentrated under reduced pressure to give the title compound (200 mg, crude) as a yellow oil. LCMS (ES⁺): m/z 381.3 [M+H]⁺.

Synthesis of cis-3-(4-(4-(2-(4-(3-((5-chloro-4-(4′-fluoro-[1,1′-biphenyl]-3-yl)pyrimidin-2-yl)amino)cyclohexane-1-carbonyl)piperazin-1-yl)-2-oxoethyl)-4-hydroxypiperidin-1-yl)-3-fluorophenoxy)piperidine-2,6-dione (Compound 292). To a solution of 2-(1-(4-((2,6-dioxopiperidin-3-yl)oxy)-2-fluorophenyl)-4-hydroxypiperidin-4-yl)acetic acid (80.0 mg, 210 μmol, 1.00 eq) and DIEA (54.4 mg, 420 μmol, 73.3 μL, 2.00 eq) in DMF (1 mL) was added HATU (120 mg, 315 μmol, 1.50 eq) and cis-(3-((5-chloro-4-(4′-fluoro-[1,1′-biphenyl]-3-yl)pyrimidin-2-yl)amino)cyclohexyl)(piperazin-1-yl)methanone (128 mg, 210 μmol, 1.00 eq, TFA) at 20 °C, the mixture was stirred at 20 °C for 12 h. LCMS showed 2-(1-(4-((2,6-dioxopiperidin-3-yl)oxy)-2-fluorophenyl)-4-hydroxypiperidin-4-yl)acetic acid was consumed and the desired mass was detected. The mixture was concentrated under reduced pressure to give a residue. This residue was purified by prep-HPLC (column: Phenomenex luna C18 100*40 mm*3 um; mobile phase: [H₂O (0.2% FA)-ACN]; gradient: 35%-70% B over 8.0 min) to give the title compound (Compound 292) (10.8 mg, 12.5 μmol, 5.93% yield, 98.9% purity) as a white solid. LCMS (ES⁺): m/z 856.3 [M+H]⁺

### Example 310. Synthesis of cis-3-((4-(1-(2-(2-(3-((5-chloro-4-(4′-fluoro-[1,1′-biphenyl]-3-yl)pyrimidin-2-yl)amino)cyclohexane-1-carbonyl)-2,6-diazaspiro[3.4]octan-6-yl)-2-oxoethyl)piperidin-4-yl)-3-fluorophenyl)amino)piperidine-2,6-dione (Compound 284)

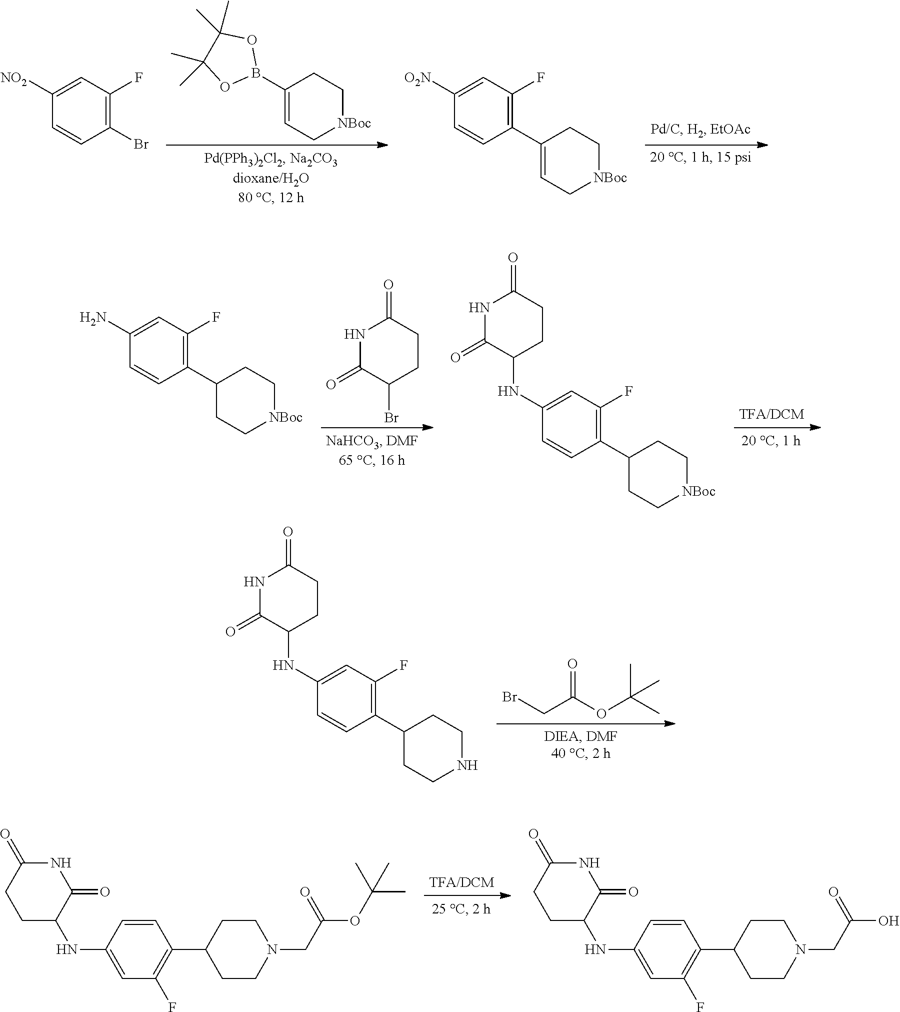

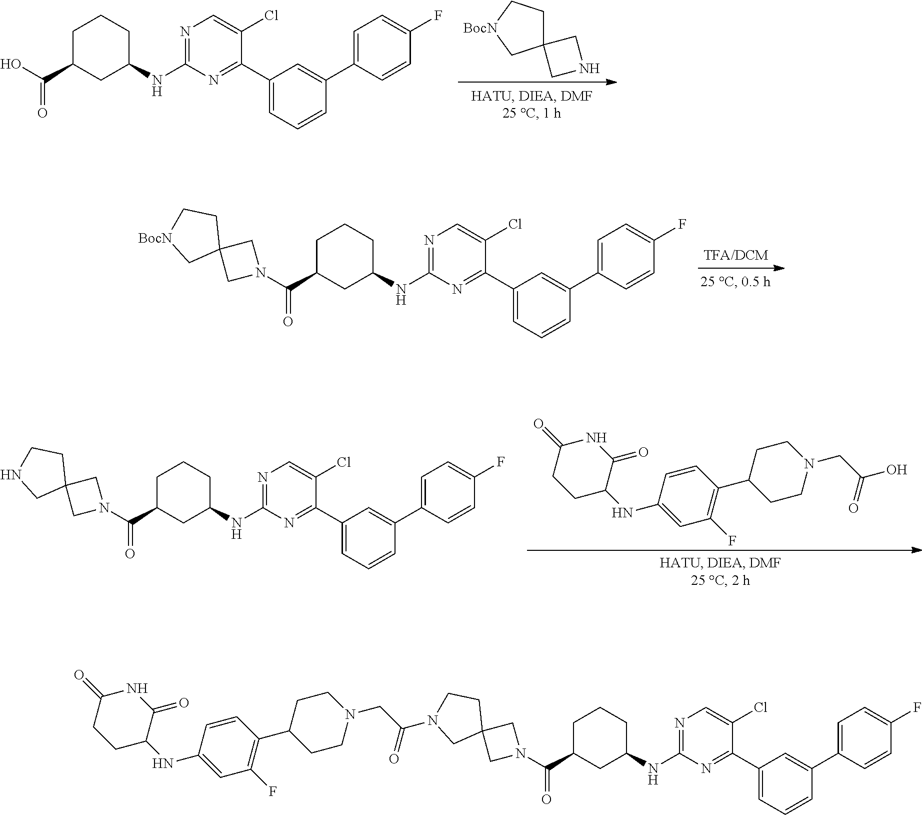 [SMILES: O=C(O)[C@H]1CCC[C@@H](Nc2ncc(Cl)c(-c3cccc(-c4ccc(F)cc4)c3)n2)C1 ; CC(C)(C)OC(=O)N1CCC2(CNC2)C1 ; CC(C)(C)OC(=O)N1CCC2(C1)CN(C(=O)[C@H]1CCC[C@@H](Nc3ncc(Cl)c(-c4cccc(-c5ccc(F)cc5)c4)n3)C1)C2 ; O=C([C@H]1CCC[C@@H](Nc2ncc(Cl)c(-c3cccc(-c4ccc(F)cc4)c3)n2)C1)N1CC2(CCNC2)C1 ; O=C(O)CN1CCC(c2ccc(NC3CCC(=O)NC3=O)cc2F)CC1 ; O=C1CCC(Nc2ccc(C3CCN(CC(=O)N4CCC5(C4)CN(C(=O)[C@H]4CCC[C@@H](Nc6ncc(Cl)c(-c7cccc(-c8ccc(F)cc8)c7)n6)C4)C5)CC3)c(F)c2)C(=O)N1]

Synthesis of tert-butyl 4-(2-fluoro-4-nitrophenyl)-3,6-dihydropyridine-1(2H)-carboxylate. To a stirred solution of 1-bromo-2-fluoro-4-nitrobenzene (2.50 g, 11.4 mmol, 1.00 eq) and tert-butyl 4-(4,4,5,5-tetramethyl-1,3,2-dioxaborolan-2-yl)-3,6-dihydropyridine-1(2H)-carboxylate (4.57 g, 14.8 mmol, 1.30 eq) in dioxane (50 mL) and H₂O (5 mL) were added Pd(PPh₃)₂Cl₂ (399 mg, 568 μmol, 0.0500 eq) and Na₂CO₃ (2.41 g, 22.7 mmol, 2.00 eq) at 20 °C under N₂. The resulting mixture was stirred at 80 °C for 12 h under N₂. LCMS showed 1-bromo-2-fluoro-4-nitrobenzene was consumed completely and the desired mass was detected. The mixture was diluted with H₂O (50 mL) and then extracted with EtOAc (50 mL*3). The combined organic layers were dried over Na₂SO₄, filtered and the filtrate was concentrated under reduced pressure to give a residue. The residue was purified by MPLC (SiO₂, PE/EtOAc=1/0 to 1/1) to give the title compound (2.00 g, 6.20 mmol, 54.6% yield) as a yellow oil. LCMS (ES⁺): m/z 308.2 [M+H−56+MeCN]⁺

Synthesis of tert-butyl 4-(4-amino-2-fluorophenyl)piperidine-1-carboxylate. To a stirred solution of tert-butyl 4-(2-fluoro-4-nitrophenyl)-3,6-dihydropyridine-1(2H)-carboxylate (2.00 g, 6.20 mmol, 1.00 eq) in EtOAc (40 mL) was added 10% Pd/C (2.00 g, 50% purity) at 20 °C under N₂. The mixture was degassed and purged with H₂ for 3 times and the resulting mixture was stirred at 20 °C for 1 h under H₂ (15 psi). LCMS showed tert-butyl 4-(2-fluoro-4-nitrophenyl)-3,6-dihydropyridine-1(2H)-carboxylate was consumed and the desired mass was detected. The mixture was filtered and the filtrate was concentrated under reduced pressure to give the title compound (1.80 g, crude) as a yellow oil. LCMS (ES⁺): m/z 239.1 [M+H−56]⁺

Synthesis of tert-butyl 4-(4-((2,6-dioxopiperidin-3-yl)amino)-2-fluorophenyl)piperidine-1-carboxylate. To a stirred solution of tert-butyl 4-(4-amino-2-fluorophenyl)piperidine-1-carboxylate (1.80 g, 6.11 mmol, 1.00 eq) and 3-bromopiperidine-2,6-dione (1.76 g, 9.17 mmol, 1.50 eq) in DMF (25 mL) was added NaHCO₃ (1.54 g, 18.3 mmol, 714 μL, 3.00 eq) at 20 °C. The resulting mixture was stirred at 65 °C for 12 h. LCMS showed tert-butyl 4-(4-amino-2-fluorophenyl)piperidine-1-carboxylate remained and the desired mass was detected. Then 3-bromopiperidine-2,6-dione (1.76 g, 9.17 mmol, 1.50 eq) was added to the mixture at 20 °C. The resulting mixture was stirred at 65 °C for another 4 h. LCMS showed tert-butyl 4-(4-amino-2-fluorophenyl)piperidine-1-carboxylate was consumed completely and the desired mass was detected. To the mixture was added H₂O (20 mL), then the mixture was filtered and the filter cake was dried under reduced pressure to give the title compound (2.15 g, crude) as a blue solid. LCMS (ES⁺): m/z 350.2 [M+H−56]⁺

Synthesis of 3-((3-fluoro-4-(piperidin-4-yl)phenyl)amino)piperidine-2,6-dione. A solution of tert-butyl 4-(4-((2,6-dioxopiperidin-3-yl)amino)-2-fluorophenyl)piperidine-1-carboxylate (1.15 g, 2.84 mmol, 1.00 eq) in DCM (10 mL) and TFA (3 mL) was stirred at 20 °C for 1 h. LCMS shows tert-butyl 4-(4-((2,6-dioxopiperidin-3-yl)amino)-2-fluorophenyl)piperidine-1-carboxylate was consumed and the desired mass was detected. The reaction mixture was concentrated under reduced pressure to give the title compound (1.20 g, crude, TFA) as a brown oil. LCMS (ES⁺): m/z 306.2 [M+H]⁺

Synthesis of tert-butyl 2-(4-(4-((2,6-dioxopiperidin-3-yl)amino)-2-fluorophenyl)piperidin-1-yl)acetate. To a solution of 3-((3-fluoro-4-(piperidin-4-yl)phenyl)amino)piperidine-2,6-dione (400 mg, 1.31 mmol, 1.00 eq) in DMF (4.00 mL) was added DIEA (169 mg, 1.31 mmol, 228 μL, 1.00 eq) and tert-butyl 2-bromoacetate (255 mg, 1.31 mmol, 193 μL, 1.00 eq) at 25 °C. The mixture was stirred at 40 °C for 2 h. LC-MS showed the desired mass was detected. The reaction diluted with H₂O 50.0 mL and combined organic layers were extracted with EtOAc (50.0 mL*3), dried over Na₂SO₄, filtered and concentrated under reduced pressure. The residue was purified by column chromatography (SiO₂, Petroleum ether:Ethyl acetate=1:0 to 3:1) to give the title compound (500 mg, 1.19 mmol, 90.9% yield) as a white oil. LCMS (ES⁺): m/z 420.2[M+H]⁺.

Synthesis of 2-(4-(4-((2,6-dioxopiperidin-3-yl)amino)-2-fluorophenyl)piperidin-1-yl)acetic acid. To a solution of tert-butyl 2-(4-(4-((2,6-dioxopiperidin-3-yl)amino)-2-fluorophenyl)piperidin-1-yl)acetate (300 mg, 715 μmol, 1.00 eq) in DCM (5.00 mL) was added TFA (81.5 mg, 715 μmol, 53.1 μL, 1.00 eq) at 25 °C. The mixture was stirred at 25 °C for 2 h. LC-MS showed the desired mass was detected. The reaction concentrated under reduced pressure to give a residue. The residue was purified by prep-HPLC (column: Phenomenex luna C18 100*40 mm*5 um; mobile phase: [H₂O (0.2% FA)-ACN]; gradient: 1%-25% B over 8.0 min) to give the title compound (200 mg, 546 μmol, 76.4% yield, 99.3% purity) as a white solid. LCMS (ES⁺): m/z 364.2 [M+H]⁺.

Synthesis of tert-butyl 2-(cis-3-((5-chloro-4-(4′-fluoro-[1,1′-biphenyl]-3-yl)pyrimidin-2-yl)amino)cyclohexane-1-carbonyl)-2,6-diazaspiro[3.4]octane-6-carboxylate. To a solution of cis-3-((5-chloro-4-(4′-fluoro-[1,1′-biphenyl]-3-yl)pyrimidin-2-yl)amino)cyclohexane-1-carboxylic acid (350 mg, 821 μmol, 1.00 eq), tert-butyl 2,6-diazaspiro[3.4]octane-6-carboxylate (174 mg, 821 μmol, 1.00 eq) in DMF (1 mL) was added HATU (624 mg, 1.64 mmol, 2.00 eq) and DIEA (318 mg, 2.47 mmol, 429 μL, 3.00 eq) at 25 °C. The mixture was stirred at 25 °C for 1 h. LC-MS showed the desired mass was detected. The reaction mixture was quenched by addition H₂O 5 mL, and then extracted with EtOAc (10 mL*3). The combined organic layers were dried over Na₂SO₄, filtered and concentrated under reduced pressure. The residue was purified by column chromatography (SiO₂, Petroleum ether/Ethyl acetate=20:1 to 0:1) to give the title compound (400 mg, 645 mol, 78.48% yield) as a yellow solid. LCMS (ES⁺): m/z 620.4[M+H]⁺.

Synthesis of cis-(3-((5-chloro-4-(4′-fluoro-[1,1′-biphenyl]-3-yl)pyrimidin-2-yl)amino)cyclohexyl)(2,6-diazaspiro[3.4]octan-2-yl)methanone. A mixture of cis-tert-butyl 2-(3-((5-chloro-4-(4′-fluoro-[1,1′-biphenyl]-3-yl)pyrimidin-2-yl)amino)cyclohexane-1-carbonyl)-2,6-diazaspiro[3.4]octane-6-carboxylate (380 mg, 612 μmol, 1.00 eq) in DCM (2 mL) was added TFA (1.54 g, 13.4 mmol, 1.00 mL, 21.9 eq) and then the mixture was stirred at 25 °C for 0.5 h. LC-MS showed the desired mass was detected. The reaction mixture was concentrated under reduced pressure to give the title compound (300 mg, crude) as a yellow oil. LCMS (ES⁺): m/z 520.2 [M+H]⁺.

Synthesis of cis-3-((4-(1-(2-(2-(3-((5-chloro-4-(4′-fluoro-[1,1′-biphenyl]-3-yl)pyrimidin-2-yl)amino)cyclohexane-1-carbonyl)-2,6-diazaspiro[3.4]octan-6-yl)-2-oxoethyl)piperidin-4-yl)-3-fluorophenyl)amino)piperidine-2,6-dione (Compound 284). To a solution of 2-(4-(4-((2,6-dioxopiperidin-3-yl)amino)-2-fluorophenyl)piperidin-1-yl)acetic acid (31.4 mg, 86.5 μmol, 1.00 eq), (3-((5-chloro-4-(4′-fluoro-[1,1′-biphenyl]-3-yl)pyrimidin-2-yl)amino)cyclohexyl)(2,6-diazaspiro[3.4]octan-2-yl)methanone (45.0 mg, 86.5 μmol, 1.00 eq) in DMF (2 mL) was added HATU (65.8 mg, 173 μmol, 2.00 eq) and DIEA (33.5 mg, 259 μmol, 45.2 μL, 3.00 eq) at 25 °C. The mixture was stirred at 25 °C for 2 h. LC-MS showed the desired mass was detected. The reaction mixture was quenched by addition H₂O (10 mL), and then extracted with EtOAc (10 mL*3). The combined organic layers were dried over Na₂SO₄, filtered and concentrated under reduced pressure. The residue was purified by prep-HPLC (column: Phenomenex luna C18 100*40 mm*5 um; mobile phase: [H₂O (0.2% FA)-ACN]; gradient: 15%-45% B over 8.0 min] to give the title compound (Compound 284) (19.3 mg, 22.0 μmol, 25.5% yield, 100% purity, 0.2 FA) as a yellow solid. LCMS (ES⁺): m/z 865.3 [M+H]⁺.

### Example 311. Synthesis of cis-3-(4-(4-(1-((3-((5-chloro-4-(4′-fluoro-[1,1′-biphenyl]-3-yl)pyrimidin-2-yl)amino)cyclohexane-1-carbonyl)piperidin-4-yl)piperazin-1-yl)-3-fluorophenyl)piperidine-2,6-dione (Compound 283)

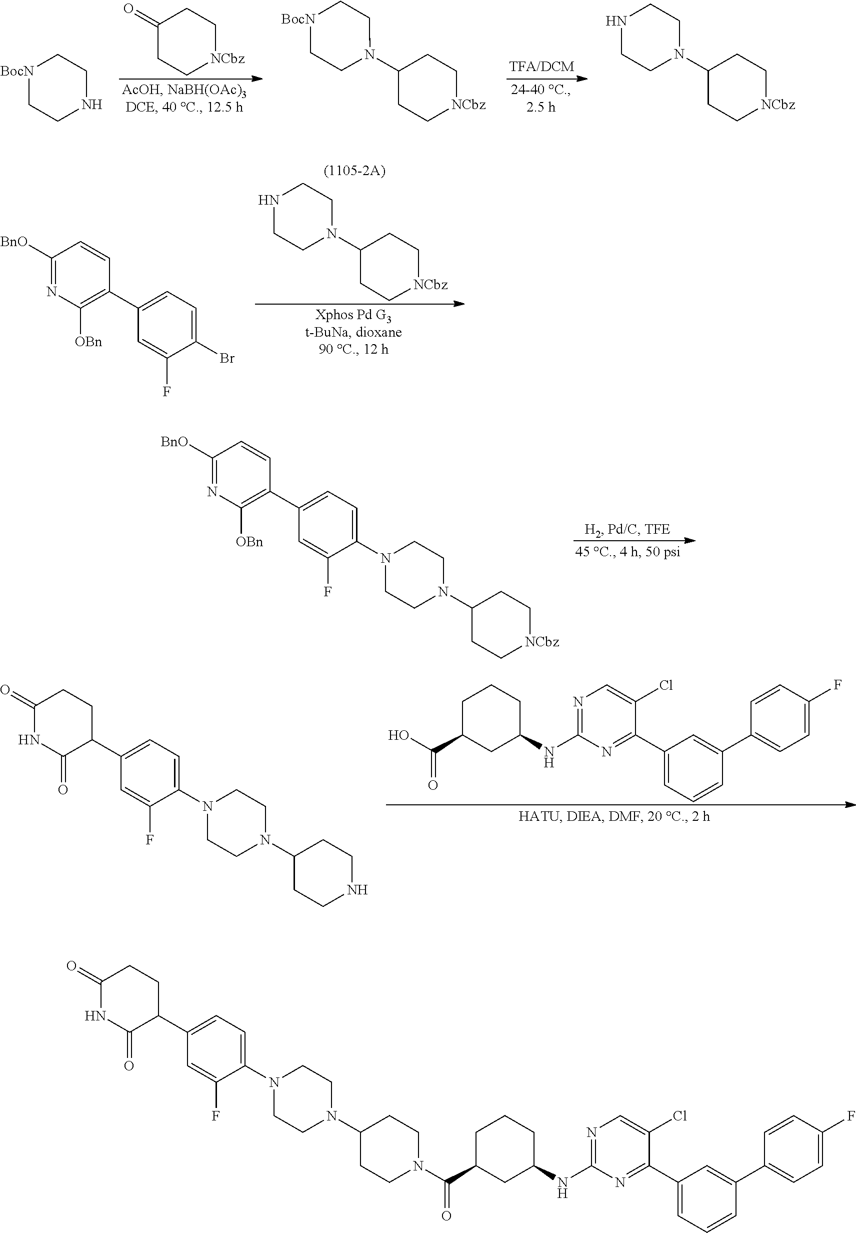

Synthesis of tert-butyl 4-(1-((benzyloxy)carbonyl)piperidin-4-yl)piperazine-1-carboxylate. To a solution of tert-butyl piperazine-1-carboxylate (10.0 g, 53.7 mmol, 1.00 eq) and benzyl 4-oxopiperidine-1-carboxylate (37.6 g, 161 mmol, 32.1 mL, 3.00 eq) in DCE (150 mL) was added AcOH (8.06 g, 134 mmol, 7.68 mL, 2.50 eq) at 25 °C. The mixture was stirred at 25 °C for 0.5 h. Then NaBH(OAc)₃ (45.5 g, 215 mmol, 4.00 eq) was added to the mixture at 25 °C. The mixture was stirred at 40 °C for 12 h. LC-MS showed the desired mass was detected. The mixture was diluted with H₂O (200 mL) and extracted with EtOAc (200 mL*3). The combined organic layers were dried over Na₂SO₄, filtered and concentrated under reduced pressure to give a residue. The residue was purified by column chromatography (SiO₂, Petroleum ether/Ethyl acetate=5/1 to 1/1) to give the title compound (21.0 g, 28.9 mmol, 26.9% yield, 55.4% purity) as a yellow oil. LCMS (ES+): m/z 404.2 [M+H]⁺.

Synthesis of benzyl 4-(piperazin-1-yl)piperidine-1-carboxylate. A solution of tert-butyl 4-(1-((benzyloxy)carbonyl)piperidin-4-yl)piperazine-1-carboxylate (7.00 g, 17.4 mmol, 1.00 eq) in DCM (50 mL) and TFA (10 mL) was stirred at 25 °C for 0.5 h. LCMS showed tert-butyl 4-(1-((benzyloxy)carbonyl)piperidin-4-yl)piperazine-1-carboxylate remained and the desired mass was detected. Then to the mixture was added TFA (15 mL) at 25 °C and the mixture was stirred at 40 °C for 2 h. LCMS showed tert-butyl 4-(1-((benzyloxy)carbonyl)piperidin-4-yl)piperazine-1-carboxylate was consumed completely and the desired mass was detected. The mixture was concentrated under reduced pressure to give a residue. The residue was purified by prep-HPLC (column: Phenomenex luna C18 (250*70 mm, 15 um); mobile phase: [H₂O (0.2% FA)-ACN]; gradient: 5%-45% B over 20.0 min) to give the title compound (3.60 g, 11.9 mmol, 68.4% yield) as a brown oil. LCMS (ES+): m/z 304.2 [M+H]⁺.

Synthesis of benzyl 4-(4-(4-(2,6-bis(benzyloxy)pyridin-3-yl)-2-fluorophenyl)piperazin-1-yl)piperidine-1-carboxylate. To a solution of 2,6-bis(benzyloxy)-3-(4-bromo-3-fluorophenyl)pyridine (1.00 g, 2.15 mmol, 1.00 eq) in dioxane (20 mL) were added benzyl 4-(piperazin-1-yl)piperidine-1-carboxylate (653 mg, 2.15 mmol, 1.00 eq), t-BuONa (621 mg, 6.46 mmol, 3.00 eq) and XPhos Pd G3 (182 mg, 215 μmol, 0.100 eq) at 20 °C under N₂ atmosphere. The mixture was stirred at 90 °C for 12 h under N₂. LCMS showed 2,6-bis(benzyloxy)-3-(4-bromo-3-fluorophenyl)pyridine was consumed completely and the desired mass was detected. The mixture was concentrated under reduced pressure. The mixture was purified by MPLC (SiO₂, PE/EtOAc=1/0 to 0/1) to give the title compound (900 mg, 1.31 mmol, 60.9% yield) as a yellow oil. LCMS (ES⁺): m/z 687.2 [M+H]⁺.

Synthesis of 3-(3-fluoro-4-(4-(piperidin-4-yl)piperazin-1-yl)phenyl)piperidine-2,6-dione. To a stirred solution of benzyl 4-(4-(4-(2,6-bis(benzyloxy)pyridin-3-yl)-2-fluorophenyl)piperazin-1-yl)piperidine-1-carboxylate (500 mg, 728 μmol, 1.00 eq) in 2,2,2-trifluoroethanol (10 mL) was added 10% Pd/C (50.0 mg, 50% purity) at 20 °C. The resulting mixture was stirred at 45 °C for 4 h under H₂ (50 psi). LCMS showed benzyl 4-(4-(4-(2,6-bis(benzyloxy)pyridin-3-yl)-2-fluorophenyl)piperazin-1-yl)piperidine-1-carboxylate was consumed and the desired mass was detected. The mixture was filtered and the filtrate was concentrated under reduced pressure to give the title compound (200 mg, crude) as a white solid. LCMS (ES⁺): m/z 375.3 [M+H]⁺.

Synthesis of cis-3-(4-(4-(1-(3-((5-chloro-4-(4′-fluoro-[1,1′-biphenyl]-3-yl)pyrimidin-2-yl)amino)cyclohexane-1-carbonyl)piperidin-4-yl)piperazin-1-yl)-3-fluorophenyl)piperidine-2,6-dione (Compound 283). To a solution of cis-3-((5-chloro-4-(4′-fluoro-[1,1′-biphenyl]-3-yl)pyrimidin-2-yl)amino)cyclohexane-1-carboxylic acid (227 mg, 534 μmol, 1.00 eq), HATU (305 mg, 801 μmol, 1.50 eq) and DIEA (138 mg, 1.07 mmol, 186 μL, 2.00 eq) in DMF (5 mL) was added 3-(3-fluoro-4-(4-(piperidin-4-yl)piperazin-1-yl)phenyl)piperidine-2,6-dione (200 mg, 534 μmol, 1.00 eq) at 20 °C, then the mixture was stirred at 20 °C for 2 h. LCMS showed 3-(3-fluoro-4-(4-(piperidin-4-yl)piperazin-1-yl)phenyl)piperidine-2,6-dione was consumed and the desired mass was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 50%-80% B over 8.0 min) to give the title compound (Compound 283) (11.1 mg, 14.0 umol, 2.62% yield, 98.8% purity) as a white solid. LCMS (ES⁺): m/z 782.2 [M+H]⁺

### Example 312. Synthesis of cis-3-(5-(1-(2-(1-(3-((5-chloro-4-(4′-fluoro-[1,1′-biphenyl]-3-yl)pyrimidin-2-yl)amino)cyclohexane-1-carbonyl)piperidin-4-yl)ethyl)piperidin-4-yl)-3-methyl-2-oxo-2,3-dihydro-1H-benzo[d]imidazol-1-yl)piperidine-2,6-dione (Compound 279)

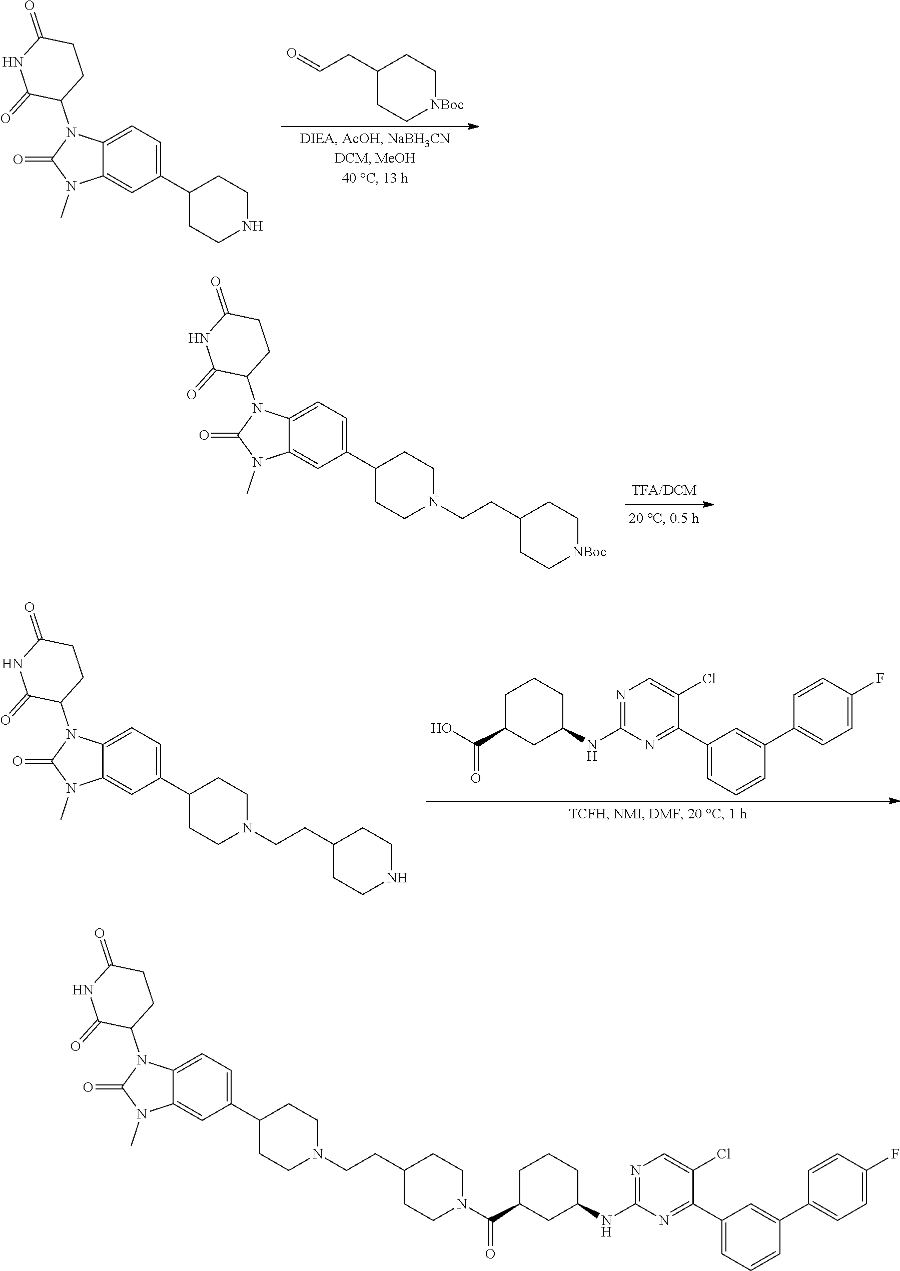

Synthesis of tert-butyl 4-(2-(4-(1-(2,6-dioxopiperidin-3-yl)-3-methyl-2-oxo-2,3-dihydro-1H-benzo[d]imidazol-5-yl)piperidin-1-yl)ethyl)piperidine-1-carboxylate. To a stirred solution of 3-(3-methyl-2-oxo-5-(piperidin-4-yl)-2,3-dihydro-1H-benzo[d]imidazol-1-yl)piperidine-2,6-dione (3.00 g, 6.57 mmol, 1.00 eq, TFA) in MeOH (30 mL) and DCM (15 mL) was added DIEA to adjust pH to 8 at 20 °C. The mixture was stirred at 20 °C for 0.5 h. Then to the mixture were added tert-butyl 4-(2-oxoethyl)piperidine-1-carboxylate (1.79 g, 7.89 mmol, 1.20 eq) and AcOH (1.19 g, 19.7 mmol, 1.13 mL, 3.00 eq) to adjust pH to 5 at 20 °C. The mixture was stirred at 40 °C for 0.5 h. Then NaBH₃CN (1.24 g, 19.7 mmol, 3.00 eq) was added the mixture at 20 °C. The resulting mixture was stirred at 40 °C for 12 h. LCMS showed 3-(3-methyl-2-oxo-5-(piperidin-4-yl)-2,3-dihydro-1H-benzo[d]imidazol-1-yl)piperidine-2,6-dione was consumed and the desired target MS was detected. H₂O (50 mL) was added to the mixture, then the mixture was extracted with DCM (50 mL*3), the combined organic layers were dried over Na₂SO₄, filtered and the filtrate was concentrated under reduced pressure to give a residue. The residue was purified by MPLC (SiO₂, Petroleum ether/Ethyl acetate=1/0 to 1/1) to give the title compound (1.60 g, 2.89 mmol, 44.0% yield) as a yellow oil. LCMS (ES⁺): m/z 554.4 [M+H]⁺

Synthesis of 3-(3-methyl-2-oxo-5-(1-(2-(piperidin-4-yl)ethyl)piperidin-4-yl)-2,3-dihydro-1H-benzo[d]imidazol-1-yl)piperidine-2,6-dione. A solution of tert-butyl 4-(2-(4-(1-(2,6-dioxopiperidin-3-yl)-3-methyl-2-oxo-2,3-dihydro-1H-benzo[d]imidazol-5-yl)piperidin-1-yl)ethyl)piperidine-1-carboxylate (900 mg, 1.63 mmol, 1.00 eq) in DCM (9 mL) and TFA (3 mL) was stirred at 20 °C for 0.5 h. LCMS showed tert-butyl 4-(2-(4-(1-(2,6-dioxopiperidin-3-yl)-3-methyl-2-oxo-2,3-dihydro-1H-benzo[d]imidazol-5-yl)piperidin-1-yl)ethyl)piperidine-1-carboxylate was consumed and the desired target MS was detected. The mixture was concentrated under reduced pressure to give the title compound (900 mg, crude, TFA) as a yellow solid. LCMS (ES⁺): m/z 454.3 [M+H]⁺

Synthesis of cis-3-(5-(1-(2-(1-(3-((5-chloro-4-(4′-fluoro-[1,1′-biphenyl]-3-yl)pyrimidin-2-yl)amino)cyclohexane-1-carbonyl)piperidin-4-yl)ethyl)piperidin-4-yl)-3-methyl-2-oxo-2,3-dihydro-1H-benzo[d]imidazol-1-yl)piperidine-2,6-dione (Compound 279). To a solution of cis-3-((5-chloro-4-(4′-fluoro-[1,1′-biphenyl]-3-yl)pyrimidin-2-yl)amino)cyclohexane-1-carboxylic acid (150 mg, 352 μmol, 1.00 eq) in DMF (4 mL) were added TCFH (119 mg, 423 μmol, 1.20 eq) and NMI (86.8 mg, 1.06 mmol, 84.3 μL, 3.00 eq) at 20 °C, then 3-(3-methyl-2-oxo-5-(1-(2-(piperidin-4-yl)ethyl)piperidin-4-yl)-2,3-dihydro-1H-benzo[d]imidazol-1-yl)piperidine-2,6-dione (200 mg, 352 μmol, 1.00 eq, TFA) was added to the mixture, the mixture was stirred at 20 °C for 1 h. LCMS showed 3-(3-methyl-2-oxo-5-(1-(2-(piperidin-4-yl)ethyl)piperidin-4-yl)-2,3-dihydro-1H-benzo[d]imidazol-1-yl)piperidine-2,6-dione was consumed and the desired target MS was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 35%-65% B over 8.0 min) to give the title compound (Compound 279) (39.9 mg, 44.4 μmol, 12.6% yield, 95.8% purity) as a white solid. LCMS (ES⁺): m/z 861.4 [M+H]⁺

### Example 313. Synthesis of trans-N-(4-((5-chloro-4-(4′-fluoro-[1,1′-biphenyl]-3-yl)pyrimidin-2-yl)amino)cyclohexyl)-1′-(4-((2,6-dioxopiperidin-3-yl)amino)phenyl)-[1,4′-bipiperidine]-4-carboxamide (Compound 285)

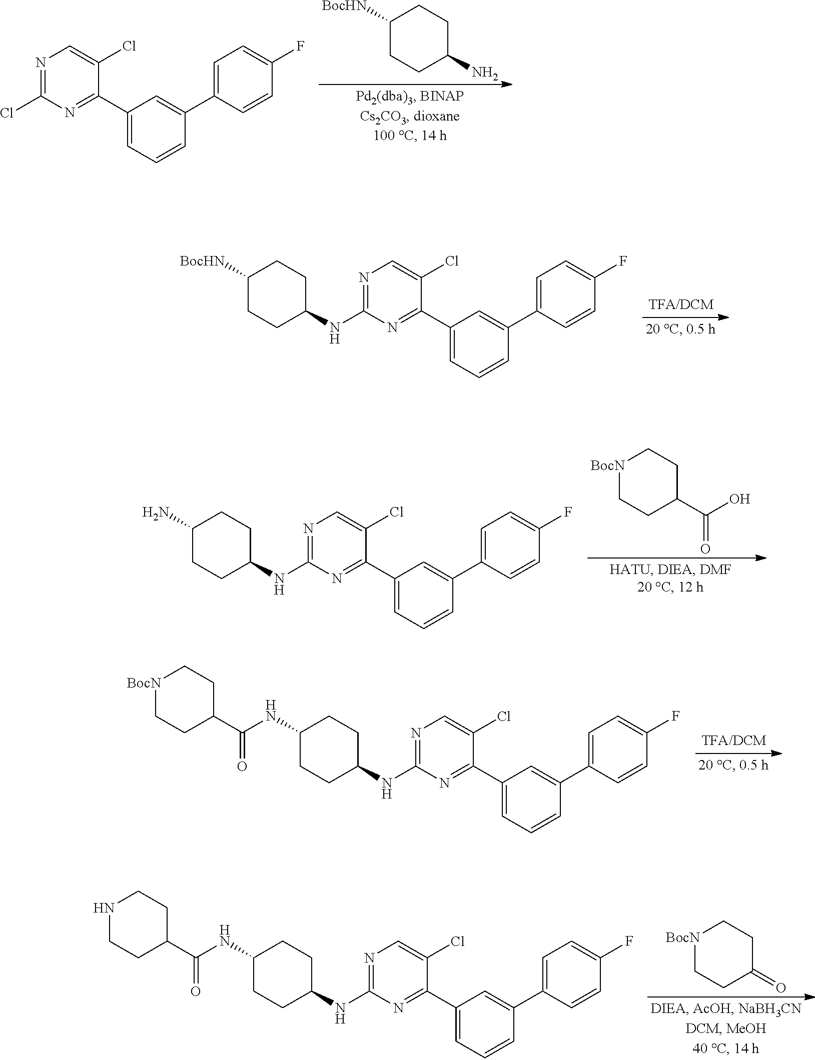

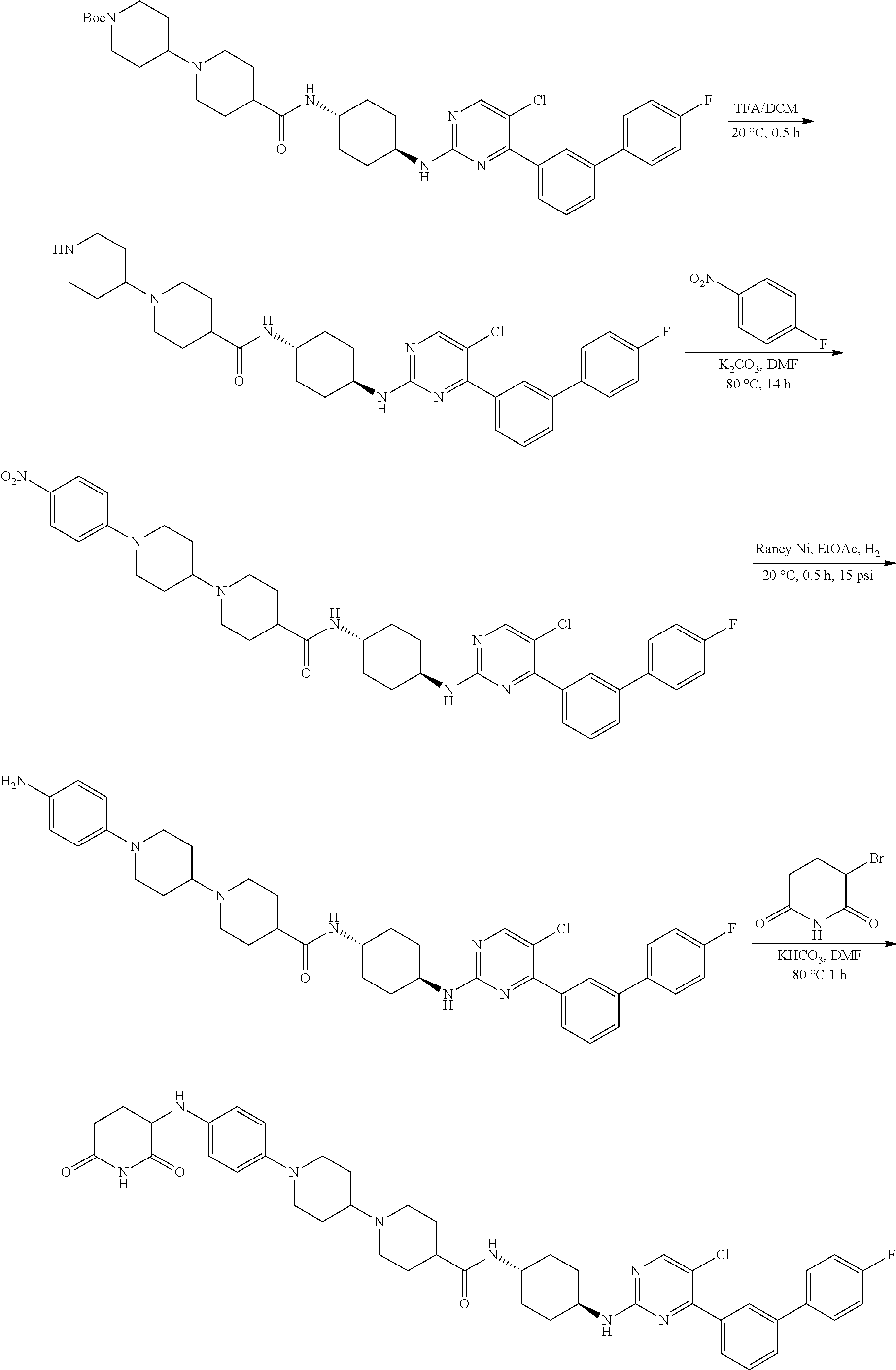

Synthesis of trans-tert-butyl (4-((5-chloro-4-(4′-fluoro-[1,1′-biphenyl]-3-yl)pyrimidin-2-yl)amino)cyclohexyl)carbamate. To a solution of 2,5-dichloro-4-(4′-fluoro-[1,1′-biphenyl]-3-yl)pyrimidine (2.00 g, 6.27 mmol, 1.00 eq) and trans-tert-butyl (4-aminocyclohexyl)carbamate (1.34 g, 6.27 mmol, 1.00 eq) in dioxane (30 mL) were added BINAP (234 mg, 376 μmol, 0.0600 eq), Pd₂(dba)₃ (230 mg, 251 μmol, 0.0400 eq) and Cs₂CO₃ (4.08 g, 12.5 mmol, 2.00 eq) at 20 °C under N₂. The mixture was stirred at 100 °C for 12 h under N₂ atmosphere. LCMS showed 2,5-dichloro-4-(4′-fluoro-[1,1′-biphenyl]-3-yl)pyrimidine remained and the desired mass was detected. Then trans-tert-butyl (4-aminocyclohexyl)carbamate (671 mg, 3.13 mmol, 0.500 eq), BINAP (117 mg, 188 μmol, 0.0300 eq), Pd₂(dba)₃ (115 mg, 125 μmol, 0.0200 eq) and Cs₂CO₃ (2.04 g, 6.27 mmol, 1.00 eq) were added to the mixture at 20 °C under N₂, the mixture was stirred at 100 °C for another 2 h under N₂ atmosphere, LCMS showed 2,5-dichloro-4-(4′-fluoro-[1,1′-biphenyl]-3-yl)pyrimidine was consumed completely, and the desired mass was detected. H₂O (30 mL) was added to the solution, the mixture was extracted with EtOAc (30 mL*3), then the combined organic layers were dried over Na₂SO₄, filtered and the filtrate was concentrated under reduced pressure to give a residue. The residue was purified by MPLC (SiO₂, PE/EtOAc=1/0 to 1/1) to give the title compound (1.50 g, 3.02 mmol, 48.2% yield) as a white solid. LCMS (ES⁺): m/z 497.2 [M+H]⁺

Synthesis of trans-N-(5-chloro-4-(4′-fluoro-[1,1′-biphenyl]-3-yl)pyrimidin-2-yl)cyclohexane-1,4-diamine. A solution of trans-tert-butyl (4-((5-chloro-4-(4′-fluoro-[1,1′-biphenyl]-3-yl)pyrimidin-2-yl)amino)cyclohexyl)carbamate (840 mg, 1.69 mmol, 1.00 eq) in DCM (9 mL) and TFA (3 mL) was stirred at 20 °C for 0.5 h. LCMS showed trans-tert-butyl (4-((5-chloro-4-(4′-fluoro-[1,1′-biphenyl]-3-yl)pyrimidin-2-yl)amino)cyclohexyl)carbamate was consumed and the desired target MS was detected. The mixture was concentrated under reduced pressure to give the title compound (840 mg, crude, TFA) as a yellow oil. LCMS (ES⁺): m/z 397.1 [M+H]⁺

Synthesis of trans-tert-butyl 4-((4-((5-chloro-4-(4′-fluoro-[1,1′-biphenyl]-3-yl)pyrimidin-2-yl)amino)cyclohexyl)carbamoyl)piperidine-1-carboxylate. To a solution of 1-(tert-butoxycarbonyl)piperidine-4-carboxylic acid (415 mg, 1.81 mmol, 1.10 eq) in DMF (10 mL) were added HATU (750 mg, 1.97 mmol, 1.20 eq) and DIEA (1.06 g, 8.22 mmol, 1.43 mL, 5.00 eq) at 20 °C, then trans-N1-(5-chloro-4-(4′-fluoro-[1,1′-biphenyl]-3-yl)pyrimidin-2-yl)cyclohexane-1,4-diamine (840 mg, 1.64 mmol, 1.00 eq, TFA) was added to the mixture, the mixture was stirred at 20 °C for 12 h. LCMS showed trans-N1-(5-chloro-4-(4′-fluoro-[1,1′-biphenyl]-3-yl)pyrimidin-2-yl)cyclohexane-1,4-diamine was consumed and the desired target MS was detected. Water (10 mL) was added to the mixture, the solid was precipitated, the mixture was filtered, the filter cake was dried under reduced pressure to give the title compound (820 mg, crude) as a white solid. LCMS (ES⁺): m/z 608.3 [M+H]⁺

Synthesis of trans-N-(4-((5-chloro-4-(4′-fluoro-[1,1′-biphenyl]-3-yl)pyrimidin-2-yl)amino)cyclohexyl)piperidine-4-carboxamide. A solution of trans-tert-butyl 4-((4-((5-chloro-4-(4′-fluoro-[1,1′-biphenyl]-3-yl)pyrimidin-2-yl)amino)cyclohexyl)carbamoyl)piperidine-1-carboxylate (800 mg, 1.32 mmol, 1.00 eq) in DCM (6 mL) and TFA (2 mL) was stirred at 20 °C for 0.5 h. LCMS showed trans-tert-butyl 4-((4-((5-chloro-4-(4′-fluoro-[1,1′-biphenyl]-3-yl)pyrimidin-2-yl)amino)cyclohexyl)carbamoyl)piperidine-1-carboxylate was consumed and the desired target MS was detected. The mixture was concentrated under reduced pressure to give the title compound (810 mg, crude, TFA) as a yellow oil. LCMS (ES⁺): m/z 508.2 [M+H]⁺

Synthesis of trans-tert-butyl 4-((4-((5-chloro-4-(4′-fluoro-[1,1′-biphenyl]-3-yl)pyrimidin-2-yl)amino)cyclohexyl)carbamoyl)-[1,4′-bipiperidine]-1′-carboxylate. To a solution of trans-N-(4-((5-chloro-4-(4′-fluoro-[1,1′-biphenyl]-3-yl)pyrimidin-2-yl)amino)cyclohexyl)piperidine-4-carboxamide (800 mg, 1.29 mmol, 1.00 eq, TFA) in DCM (10 mL) and MeOH (10 mL) was added DIEA to adjust pH to 8 at 20 °C. The mixture was stirred at 20 °C for 1 h. To the reaction were added tert-butyl 4-oxopiperidine-1-carboxylate (5.12 g, 25.7 mmol, 20.0 eq) and CH₃COOH (386 mg, 6.43 mmol, 368 μL, 5.00 eq) at 20 °C. The mixture was stirred at 40 °C for 1 h. Then NaBH₃CN (566 mg, 9.00 mmol, 7.00 eq) was added to the mixture. The mixture was stirred at 40 °C for 12 h. LCMS showed trans-N-(4-((5-chloro-4-(4′-fluoro-[1,1′-biphenyl]-3-yl)pyrimidin-2-yl)amino)cyclohexyl)piperidine-4-carboxamide was consumed and the desired target MS was detected. The mixture was concentrated under reduced pressure to give a residue. H₂O (20 mL) was added to the residue, the mixture was extracted with EtOAc (20 mL*3), the combined organic lays were dried over Na₂SO₄, filtered and the filtrate was concentrated under reduced pressure to give a residue. The residue was purified by MPLC (SiO₂, DCM/MeOH=1/0 to 10/1) to give the title compound (880 mg, 1.27 mmol, 99.0% yield) as a white solid. LCMS (ES⁺): m/z 691.4 [M+H]⁺

Synthesis of trans-N-(4-((5-chloro-4-(4′-fluoro-[1,1′-biphenyl]-3-yl)pyrimidin-2-yl)amino)cyclohexyl)-[1,4′-bipiperidine]-4-carboxamide. A solution of trans-tert-butyl 4-((4-((5-chloro-4-(4′-fluoro-[1,1′-biphenyl]-3-yl)pyrimidin-2-yl)amino)cyclohexyl)carbamoyl)-[1,4′-bipiperidine]-1′-carboxylate (880 mg, 1.27 mmol, 1.00 eq) in DCM (12 mL) and TFA (4 mL) was stirred at 20 °C for 0.5 h. LCMS showed 4-((4-((5-chloro-4-(4′-fluoro-[1,1′-biphenyl]-3-yl)pyrimidin-2-yl)amino)cyclohexyl)carbamoyl)-[1,4′-bipiperidine]-1′-carboxylate was consumed and the desired target MS was detected. The mixture was concentrated under reduced pressure to give the title compound (880 mg, crude, TFA) as a yellow oil. LCMS (ES⁺): m/z 591.3 [M+H]⁺

Synthesis of trans-N-(4-((5-chloro-4-(4′-fluoro-[1,1′-biphenyl]-3-yl)pyrimidin-2-yl)amino)cyclohexyl)-1′-(4-nitrophenyl)-[1,4′-bipiperidine]-4-carboxamide. To a stirred solution of trans-N-(4-((5-chloro-4-(4′-fluoro-[1,1′-biphenyl]-3-yl)pyrimidin-2-yl)amino)cyclohexyl)-[1,4′-bipiperidine]-4-carboxamide (880 mg, 1.25 mmol, 1.00 eq, TFA) and K₂CO₃ (517 mg, 3.74 mmol, 3.00 eq) in DMF (10 mL) was added 1-fluoro-4-nitrobenzene (264 mg, 1.87 mmol, 199 μL, 1.50 eq) at 20 °C. The resulting mixture was stirred at 80 °C for 2 h. LCMS showed trans-N-(4-((5-chloro-4-(4′-fluoro-[1,1′-biphenyl]-3-yl)pyrimidin-2-yl)amino)cyclohexyl)-[1,4′-bipiperidine]-4-carboxamide remained and the desired target MS was detected. Then the mixture was stirred at 80 °C for another 12 h, LCMS showed trans-N-(4-((5-chloro-4-(4′-fluoro-[1,1′-biphenyl]-3-yl)pyrimidin-2-yl)amino)cyclohexyl)-[1,4′-bipiperidine]-4-carboxamide was consumed completely and the desired target MS was detected. The mixture was poured into H₂O (8 mL), the mixture was filtered and the filter cake was dried under reduced pressure to give the title compound (530 mg, crude) as a yellow solid. LCMS (ES⁺): m/z 712.3 [M+H]⁺

Synthesis of trans-1′-(4-aminophenyl)-N-(4-((5-chloro-4-(4′-fluoro-[1,1′-biphenyl]-3-yl)pyrimidin-2-yl)amino)cyclohexyl)-[1,4′-bipiperidine]-4-carboxamide. To a mixture of trans-N-(4-((5-chloro-4-(4′-fluoro-[1,1′-biphenyl]-3-yl)pyrimidin-2-yl)amino)cyclohexyl)-1′-(4-nitrophenyl)-[1,4′-bipiperidine]-4-carboxamide (170 mg, 239 μmol, 1.00 eq) in EtOAc (3 mL) was added Raney-Ni (170 mg) at 20 °C, then the mixture was stirred at 20 °C for 0.5 h under H₂ at 15 psi. LCMS showed trans-N-(4-((5-chloro-4-(4′-fluoro-[1,1′-biphenyl]-3-yl)pyrimidin-2-yl)amino)cyclohexyl)-1′-(4-nitrophenyl)-[1,4′-bipiperidine]-4-carboxamide was consumed and the desired target MS was detected. The mixture was filtered and the filtrate was concentrated under reduced pressure to give the title compound (100 mg, crude) as a yellow oil. LCMS (ES⁺): m/z 682.4 [M+H]⁺

Synthesis of trans-N-(4-((5-chloro-4-(4′-fluoro-[1,1′-biphenyl]-3-yl)pyrimidin-2-yl)amino)cyclohexyl)-1′-(4-((2,6-dioxopiperidin-3-yl)amino)phenyl)-[1,4′-bipiperidine]-4-carboxamide (Compound 285). To a stirred solution of trans-1′-(4-aminophenyl)-N-(4-((5-chloro-4-(4′-fluoro-[1,1′-biphenyl]-3-yl)pyrimidin-2-yl)amino)cyclohexyl)-[1,4′-bipiperidine]-4-carboxamide (100 mg, 147 μmol, 1.00 eq) and 3-bromopiperidine-2,6-dione (84.4 mg, 440 μmol, 3.00 eq) in DMF (1 mL) was added KHCO₃ (44.0 mg, 440 μmol, 3.00 eq) at 20 °C. The resulting mixture was stirred at 80 °C for 1 h. LCMS showed trans-1′-(4-aminophenyl)-N-(4-((5-chloro-4-(4′-fluoro-[1,1′-biphenyl]-3-yl)pyrimidin-2-yl)amino)cyclohexyl)-[1,4′-bipiperidine]-4-carboxamide was consumed mostly and the desired target MS was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 10%-45% B over 8.0 min) to give the title compound (Compound 285) (11.1 mg, 13.7 μmol, 9.35% yield, 98.0% purity) as a purple solid. LCMS (ES⁺): m/z 793.4 [M+H]⁺

### Example 314. Synthesis of trans-N-(4-((5-chloro-4-(4′-fluoro-[1,1′-biphenyl]-3-yl)pyrimidin-2-yl)amino)cyclohexyl)-2-(4-((4-(1-(2,6-dioxopiperidin-3-yl)-3-methyl-2-oxo-2,3-dihydro-1H-benzo[d]imidazol-5-yl)piperidin-1-yl)methyl)piperidin-1-yl)acetamide (Compound 276)

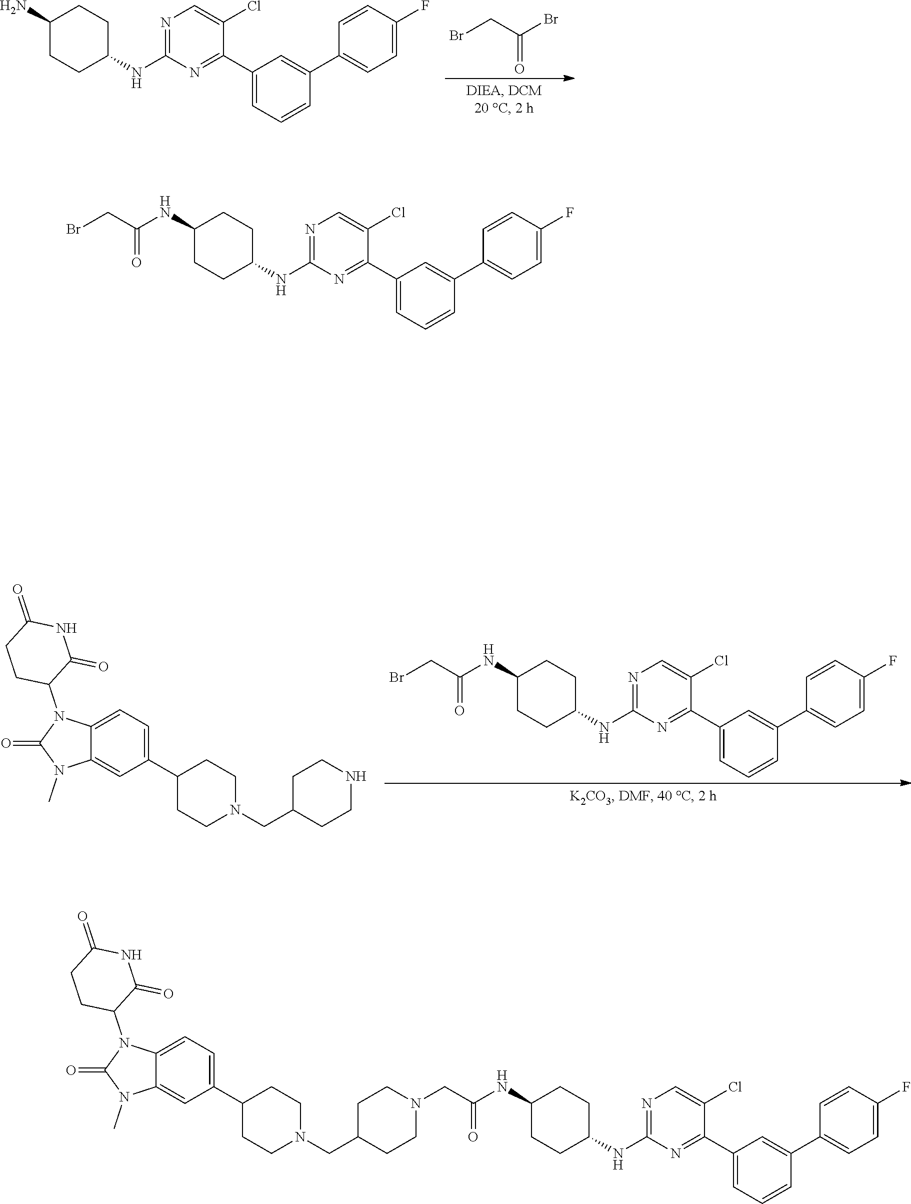 [SMILES: N[C@H]1CC[C@H](Nc2ncc(Cl)c(-c3cccc(-c4ccc(F)cc4)c3)n2)CC1 ; O=C(Br)CBr ; O=C(CBr)N[C@H]1CC[C@H](Nc2ncc(Cl)c(-c3cccc(-c4ccc(F)cc4)c3)n2)CC1 ; Cn1c(=O)n(C2CCC(=O)NC2=O)c2ccc(C3CCN(CC4CCNCC4)CC3)cc21 ; O=C(CBr)N[C@H]1CC[C@H](Nc2ncc(Cl)c(-c3cccc(-c4ccc(F)cc4)c3)n2)CC1 ; Cn1c(=O)n(C2CCC(=O)NC2=O)c2ccc(C3CCN(CC4CCN(CC(=O)N[C@H]5CC[C@H](Nc6ncc(Cl)c(-c7cccc(-c8ccc(F)cc8)c7)n6)CC5)CC4)CC3)cc21]

Synthesis of trans-2-bromo-N-(4-((5-chloro-4-(4′-fluoro-[1,1′-biphenyl]-3-yl)pyrimidin-2-yl)amino)cyclohexyl)acetamide. To a solution of trans-N¹-(5-chloro-4-(4′-fluoro-[1,1′-biphenyl]-3-yl)pyrimidin-2-yl)cyclohexane-1,4-diamine (340 mg, 785 μmol, 1.00 eq, HCl) in DCM (5 mL) were added DIEA (304 mg, 2.35 mmol, 410 μL, 3.00 eq) and 2-bromoacetyl bromide (190 mg, 942 μmol, 82.0 μL, 1.20 eq) at 20 °C. The mixture was stirred at 20 °C for 1 h. LCMS showed ~48.8% of trans-N¹-(5-chloro-4-(4′-fluoro-[1,1′-biphenyl]-3-yl)pyrimidin-2-yl)cyclohexane-1,4-diamine was remaining and desired mass was detected. Then the mixture was stirred at 20 °C for 1 h. LCMS showed ~11.1% of trans-N¹-(5-chloro-4-(4′-fluoro-[1,1′-biphenyl]-3-yl)pyrimidin-2-yl)cyclohexane-1,4-diamine was remaining and desired mass was detected. The reaction mixture was concentrated under reduced pressure to remove solvent. The residue was purified by prep-TLC (SiO₂, Petroleum ether:Ethyl acetate=1:1) to give the title compound (220 mg, 425 μmol, 54.2% yield) as a yellow solid. LCMS (ES⁺): m/z 517.1 [M+H]⁺.

Synthesis of trans-N-(4-((5-chloro-4-(4′-fluoro-[1,1′-biphenyl]-3-yl)pyrimidin-2-yl)amino)cyclohexyl)-2-(4-((4-(1-(2,6-dioxopiperidin-3-yl)-3-methyl-2-oxo-2,3-dihydro-1H-benzo[d]imidazol-5-yl)piperidin-1-yl)methyl)piperidin-1-yl)acetamide (Compound 276). To a solution of trans-2-bromo-N-(4-((5-chloro-4-(4′-fluoro-[1,1′-biphenyl]-3-yl)pyrimidin-2-yl)amino)cyclohexyl)acetamide (101 mg, 195 μmol, 1.00 eq) in DMF (3 mL) were added K₂CO₃ (135 mg, 975 μmol, 5.00 eq) and 3-(3-methyl-2-oxo-5-(1-(piperidin-4-ylmethyl)piperidin-4-yl)-2,3-dihydro-1H-benzo[d]imidazol-1-yl)piperidine-2,6-dione (108 mg, 195 μmol, 1.00 eq, TFA) at 20 °C. The mixture was stirred at 40 °C for 2 h. LCMS showed 3-(3-methyl-2-oxo-5-(1-(piperidin-4-ylmethyl)piperidin-4-yl)-2,3-dihydro-1H-benzo[d]imidazol-1-yl)piperidine-2,6-dione was consumed completely and the desired mass was detected. The organic layers were filtered and the filtrate was concentrated under reduced pressure to give a residue. The residue was purified by prep-HPLC (column: Phenomenex Luna C18 100*30 mm*3 um; mobile phase: [H₂O (0.2% FA)-ACN]; gradient: 15%-55% B over 8.0 min) to give the title compound (Compound 276) (28.3 mg, 30.7 μmol, 15.7% yield, 100% purity, FA) as a white solid. LCMS (ES⁺): m/z 876.4 [M+H]⁺.

### Example 315. Synthesis of cis-3-(5-(1-(2-(1-(3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-3-methyl-2-oxo-2,3-dihydro-1H-benzo[d]imidazol-1-yl)piperidine-2,6-dione (Compound 291)

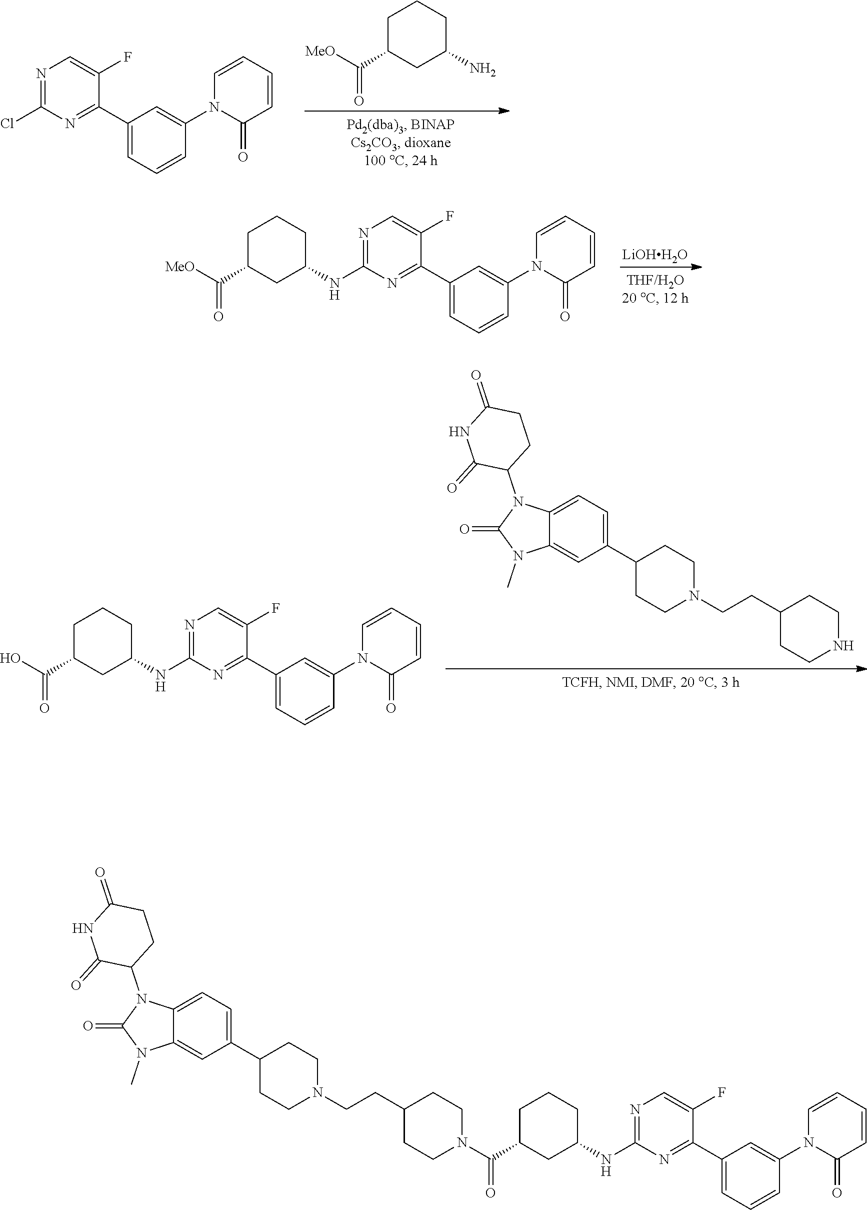

Synthesis of cis-methyl 3-((5-fluoro-4-(3-(2-oxopyridin-1(2H)-yl)phenyl)pyrimidin-2-yl)amino)cyclohexane-1-carboxylate. To a solution of 1-(3-(2-chloro-5-fluoropyrimidin-4-yl)phenyl)pyridin-2(1H)-one (730 mg, 2.42 mmol, 1.00 eq) and cis-methyl 3-aminocyclohexane-1-carboxylate (380 mg, 2.42 mmol, 1.00 eq) in dioxane (10 mL) were added Cs₂CO₃ (1.58 g, 4.84 mmol, 2.00 eq), BINAP (90.4 mg, 145 μmol, 0.0600 eq) and Pd₂(dba)₃ (88.6 mg, 96.8 μmol, 0.0400 eq) at 20 °C under N₂, the mixture was stirred at 100 °C for 12 h under N₂ atmosphere. LCMS showed 1-(3-(2-chloro-5-fluoropyrimidin-4-yl)phenyl)pyridin-2(1H)-one remained and the desired target MS was detected. Then cis-methyl 3-aminocyclohexane-1-carboxylate (380 mg, 2.42 mmol, 1.00 eq), Cs₂CO₃ (1.58 g, 4.84 mmol, 2.00 eq), BINAP (90.4 mg, 145.2 μmol, 0.0600 eq) and Pd₂(dba)₃ (88.6 mg, 96.8 μmol, 0.0400 eq) were added to the mixture at 20 °C under N₂, the mixture was stirred at 100 °C for another 12 h under N₂ atmosphere. LCMS showed 1-(3-(2-chloro-5-fluoropyrimidin-4-yl)phenyl)pyridin-2(1H)-one was consumed completely and the desired target MS was detected. H₂O (20 mL) was added to the solution, then the mixture was extracted with EtOAc (20 mL*3), the combined organic layers were dried over Na₂SO₄, filtered and the filtrate was concentrated under reduced pressure to give a residue. The residue was purified by MPLC (SiO₂, Petroleum ether/Ethyl acetate=1/0 to 1/1) to give the title compound (600 mg, 1.42 mmol, 58.7% yield) as a white solid. LCMS (ES⁺): m/z 423.2 [M+H]⁺

Synthesis of cis-3-((5-fluoro-4-(3-(2-oxopyridin-1(2H)-yl)phenyl)pyrimidin-2-yl)amino)cyclohexane-1-carboxylic acid. To a solution of cis-methyl 3-((5-fluoro-4-(3-(2-oxopyridin-1(2H)-yl)phenyl)pyrimidin-2-yl)amino)cyclohexane-1-carboxylate (200 mg, 473 μmol, 1.00 eq) in THF (2 mL) and H₂O (1 mL) was added LiOH·H₂O (39.7 mg, 946 μmol, 2.00 eq) at 20 °C, the mixture was stirred at 20 °C for 12 h. LCMS showed cis-methyl 3-((5-fluoro-4-(3-(2-oxopyridin-1(2H)-yl)phenyl)pyrimidin-2-yl)amino)cyclohexane-1-carboxylate was consumed and the desired target MS was detected. The reaction was concentrated under reduced pressure to remove THF. H₂O (2 mL) was added to the mixture, the aqueous phase was adjusted pH to 4 by HCl (1M), a solid was precipitated, the mixture was filtered and the filter cake was dried under reduced pressure to give the title compound (160 mg, crude) as a white solid. LCMS (ES⁺): m/z 409.1 [M+H]⁺

Synthesis of cis-3-(5-(1-(2-(1-(3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-3-methyl-2-oxo-2,3-dihydro-1H-benzo[d]imidazol-1-yl)piperidine-2,6-dione (Compound 291). To a solution of cis-3-((5-fluoro-4-(3-(2-oxopyridin-1(2H)-yl)phenyl)pyrimidin-2-yl)amino)cyclohexane-1-carboxylic acid (130 mg, 318 μmol, 1.00 eq) in DMF (4 mL) were added TCFH (107 mg, 382 μmol, 1.20 eq) and NMI (78.4 mg, 955 μmol, 76.1 μL, 3.00 eq) at 20 °C, then 3-(3-methyl-2-oxo-5-(1-(2-(piperidin-4-yl)ethyl)piperidin-4-yl)-2,3-dihydro-1H-benzo[d]imidazol-1-yl)piperidine-2,6-dione (181 mg, 318 μmol, 1.00 eq, TFA) was added to the mixture, the mixture was stirred at 20 °C for 1 h. LCMS showed cis-3-((5-fluoro-4-(3-(2-oxopyridin-1(2H)-yl)phenyl)pyrimidin-2-yl)amino)cyclohexane-1-carboxylic acid remained and the desired target MS was detected. Then 3-(3-methyl-2-oxo-5-(1-(2-(piperidin-4-yl)ethyl)piperidin-4-yl)-2,3-dihydro-1H-benzo[d]imidazol-1-yl)piperidine-2,6-dione (72.3 mg, 127 μmol, 0.400 eq, TFA), TCFH (53.6 mg, 191 μmol, 0.600 eq) and NMI (78.4 mg, 955 μmol, 76.1 μL, 3.00 eq) were added to the mixture, the mixture was stirred at 20 °C for another 2 h. LCMS showed cis-3-((5-fluoro-4-(3-(2-oxopyridin-1(2H)-yl)phenyl)pyrimidin-2-yl)amino)cyclohexane-1-carboxylic acid was consumed completely and the desired target MS was detected. The mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 5%-35% B over 8.0 min) to give the title compound (Compound 291) (51.5 mg, 60.7 μmol, 19.1% yield, 99.5% purity) as a white solid. LCMS (ES⁺): m/z 844.4 [M+H]⁺

### Example 316. Synthesis of cis-3-(6-(1-(2-(1-(3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 295)

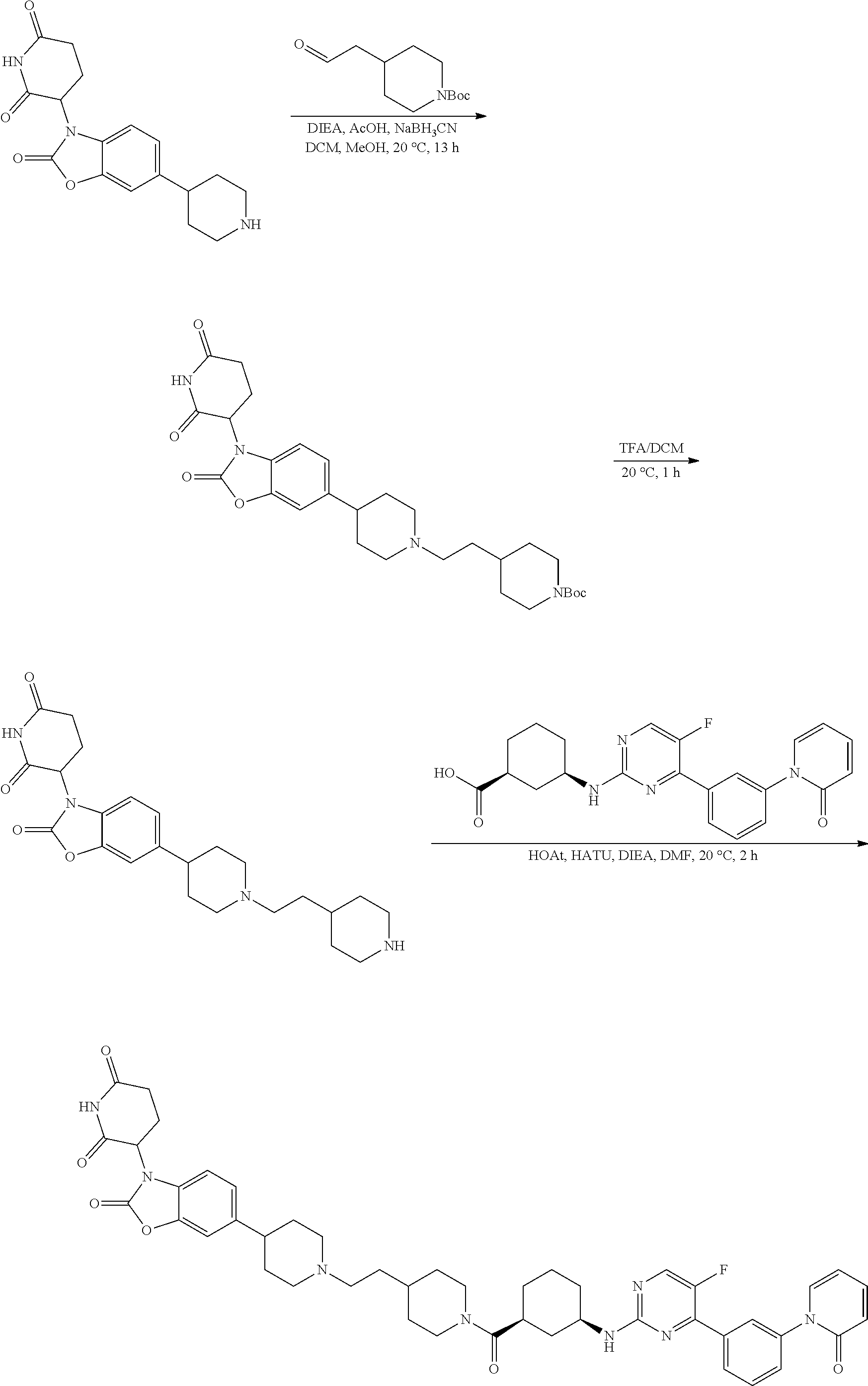 [SMILES: O=C1CCC(n2c(=O)oc3cc(C4CCNCC4)ccc32)C(=O)N1 ; CC(C)(C)OC(=O)N1CCC(CC=O)CC1 ; CC(C)(C)OC(=O)N1CCC(CCN2CCC(c3ccc4c(c3)oc(=O)n4C3CCC(=O)NC3=O)CC2)CC1 ; O=C1CCC(n2c(=O)oc3cc(C4CCN(CCC5CCNCC5)CC4)ccc32)C(=O)N1 ; O=C(O)[C@H]1CCC[C@@H](Nc2ncc(F)c(-c3cccc(-n4ccccc4=O)c3)n2)C1 ; O=C1CCC(n2c(=O)oc3cc(C4CCN(CCC5CCN(C(=O)[C@H]6CCC[C@@H](Nc7ncc(F)c(-c8cccc(-n9ccccc9=O)c8)n7)C6)CC5)CC4)ccc32)C(=O)N1]

Synthesis of tert-butyl 4-(2-(4-(3-(2,6-dioxopiperidin-3-yl)-2-oxo-2,3-dihydrobenzo[d]oxazol-6-yl)piperidin-1-yl)ethyl)piperidine-1-carboxylate. To a solution of 3-(2-oxo-6-(piperidin-4-yl)benzo[d]oxazol-3 (2H)-yl)piperidine-2,6-dione (1.00 g, 2.26 mmol, 1.00 eq, TFA) in DCM (10 mL) and MeOH (10 mL) was added DIEA to adjust pH to 8 at 20 °C. The mixture was stirred at 20 °C for 0.5 h. AcOH (135 mg, 2.26 mmol, 129 μL, 1.00 eq) and tert-butyl 4-(2-oxoethyl)piperidine-1-carboxylate (769 mg, 3.38 mmol, 1.50 eq) were added to the mixture at 20 °C. The mixture was stirred at 20 °C for 0.5 h, pH was 4-5 now. Then NaBH₃CN (709 mg, 11.3 mmol, 5.00 eq) was added to the mixture at 20 °C. The mixture was stirred at 20 °C for 12 h. LCMS showed the desired mass was detected. The reaction mixture was concentrated under reduced pressure to give a residue. The residue was purified by column chromatography (SiO₂, Petroleum ether:Ethyl acetate=1:3 to 0:1) to give the title compound (1.00 g, 1.85 mmol, 82.0% yield) as a colorless oil. LCMS (ES⁺): m/z 541.4 [M+H]⁺.

Synthesis of 3-(2-oxo-6-(1-(2-(piperidin-4-yl)ethyl)piperidin-4-yl)benzo[d]oxazol-3 (2H)-yl)piperidine-2,6-dione. To a solution of tert-butyl 4-(2-(4-(3-(2,6-dioxopiperidin-3-yl)-2-oxo-2,3-dihydrobenzo[d]oxazol-6-yl)piperidin-1-yl)ethyl)piperidine-1-carboxylate (300 mg, 555 μmol, 1.00 eq) in DCM (3 mL) was added TFA (1 mL) at 20 °C. The mixture was stirred at 20 °C for 1 h. LCMS showed tert-butyl 4-(2-(4-(3-(2,6-dioxopiperidin-3-yl)-2-oxo-2,3-dihydrobenzo[d]oxazol-6-yl)piperidin-1-yl)ethyl)piperidine-1-carboxylate was consumed completely and the desired mass was detected. The reaction mixture was concentrated under reduced pressure to give the title compound (300 mg, crude, TFA) as a yellow oil. LCMS (ES⁺): m/z 441.3 [M+H]⁺.

Synthesis of cis-3-(6-(1-(2-(1-(3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 295). To a solution of cis-3-((5-fluoro-4-(3-(2-oxopyridin-1(2H)-yl)phenyl)pyrimidin-2-yl)amino)cyclohexane-1-carboxylic acid (147 mg, 361 μmol, 1.00 eq) in DMF (3 mL) were added HOAt (24.5 mg, 180 μmol, 25.2 μL, 0.500 eq), HATU (165 mg, 433 μmol, 1.20 eq), DIEA (233 mg, 1.80 mmol, 314 μL, 5.00 eq) and 3-(2-oxo-6-(1-(2-(piperidin-4-yl)ethyl)piperidin-4-yl)benzo[d]oxazol-3 (2H)-yl)piperidine-2,6-dione (200 mg, 361 μmol, 1.00 eq, TFA) at 20 °C. The mixture was stirred at 20 °C for 2 h. LCMS showed 3-(2-oxo-6-(1-(2-(piperidin-4-yl)ethyl)piperidin-4-yl)benzo[d]oxazol-3 (2H)-yl)piperidine-2,6-dione was consumed completely and the desired mass was detected. The reaction mixture was filtered and the filtrate was concentrated under reduced pressure to give a residue. The residue was purified by prep-HPLC (column: Phenomenex Luna C18 100*30 mm*3 um; mobile phase: [H₂O (0.2% FA)-ACN]; gradient: 15%-55% B over 8.0 min) to give the title compound (Compound 295) (14.0 mg, 16.4 μmol, 4.56% yield, 97.6% purity) as a white solid. LCMS (ES⁺): m/z 831.3 [M+H]⁺.

### Example 317. Synthesis of 3-(6-(1-(2-(1-(cis-3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-1-methyl-1H-indazol-3-yl)piperidine-2,6-dione (Compound 298)

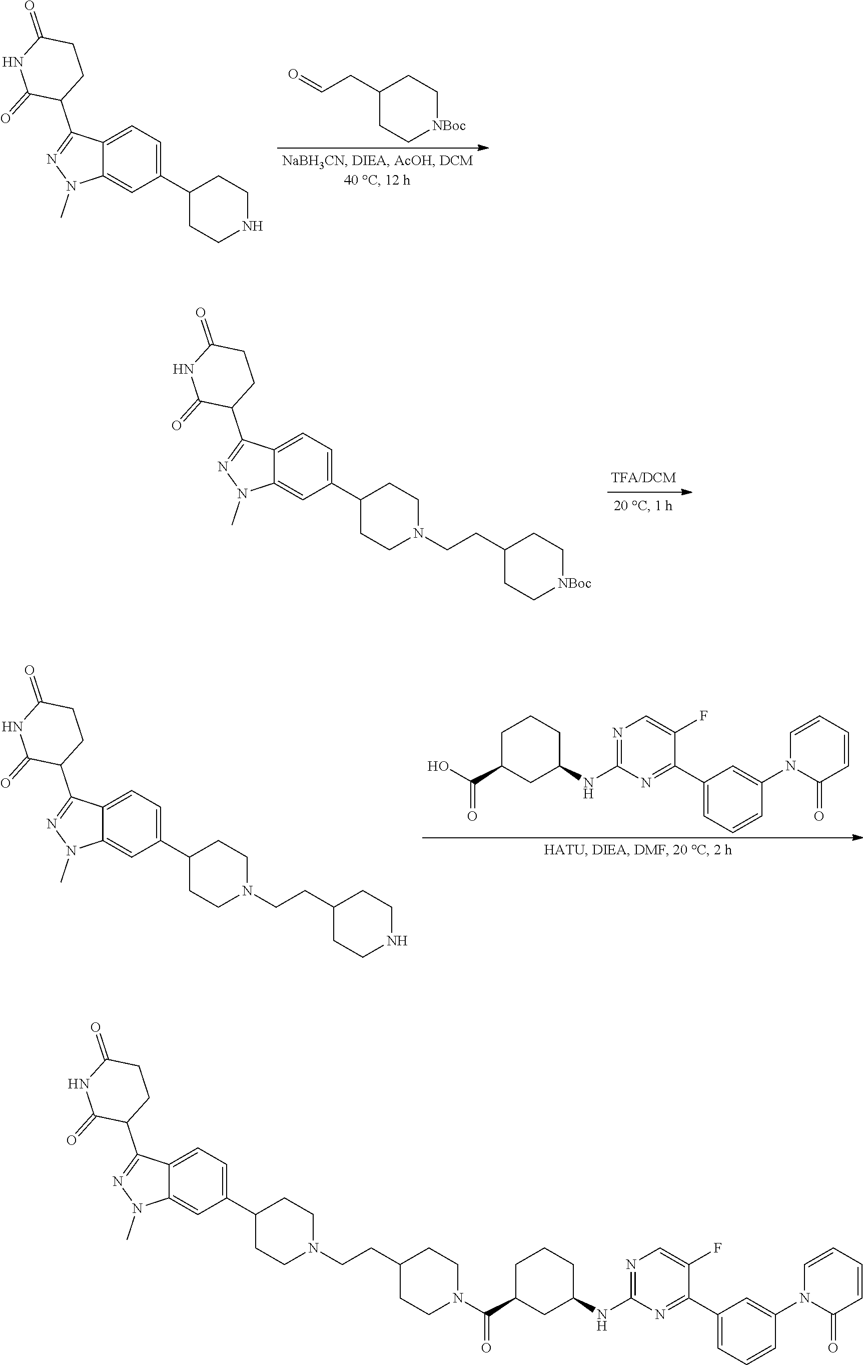 [SMILES: Cn1nc(C2CCC(=O)NC2=O)c2ccc(C3CCNCC3)cc21 ; CC(C)(C)OC(=O)N1CCC(CC=O)CC1 ; Cn1nc(C2CCC(=O)NC2=O)c2ccc(C3CCN(CCC4CCN(C(=O)OC(C)(C)C)CC4)CC3)cc21 ; Cn1nc(C2CCC(=O)NC2=O)c2ccc(C3CCN(CCC4CCNCC4)CC3)cc21 ; O=C(O)[C@H]1CCC[C@@H](Nc2ncc(F)c(-c3cccc(-n4ccccc4=O)c3)n2)C1 ; Cn1nc(C2CCC(=O)NC2=O)c2ccc(C3CCN(CCC4CCN(C(=O)[C@H]5CCC[C@@H](Nc6ncc(F)c(-c7cccc(-n8ccccc8=O)c7)n6)C5)CC4)CC3)cc21]

Synthesis of tert-butyl 4-(2-(4-(3-(2,6-dioxopiperidin-3-yl)-1-methyl-1H-indazol-6-yl)piperidin-1-yl)ethyl)piperidine-1-carboxylate. To a solution of 3-(1-methyl-6-(piperidin-4-yl)-1H-indazol-3-yl)piperidine-2,6-dione (700 mg, 2.14 mmol, 1.00 eq) in DCM (15 mL) was added DIEA (1.39 g, 10.7 mmol, 1.87 mL, 5 eq), the mixture was stirred at 20 °C for 0.5 h, then AcOH (643 mg, 10.7 mmol, 613 μL, 5.00 eq) and tert-butyl 4-(2-oxoethyl)piperidine-1-carboxylate (536 mg, 2.36 mmol, 1.10 eq) was added to the mixture at 20 °C. The mixture was stirred at 40 °C for 2 h, then NaBH₃CN (673 mg, 10.7 mmol, 5.00 eq) was added to the mixture at 40 °C. The mixture was stirred at 40 °C for 9.5 h. LC-MS showed the desired mass was detected. The reaction mixture was diluted with H₂O 50 mL and extracted with EtOAc 40 mL*3. The combined organic layers were dried over Na₂SO₄, filtered and concentrated under reduced pressure. The residue was purified by column chromatography (SiO₂, PE:EtOAc=1:0 to 0:1) to give the title compound (1 g, crude) as a yellow oil. LCMS (ES⁺): m/z 538.5 [M+H]⁺.

Synthesis of 3-(1-methyl-6-(1-(2-(piperidin-4-yl)ethyl)piperidin-4-yl)-1H-indazol-3-yl)piperidine-2,6-dione. To a solution of tert-butyl 4-[2-[4-[3-(2,6-dioxo-3-piperidyl)-1-methyl-indazol-6-yl]-1-piperidyl]ethyl]piperidine-1-carboxylate (300 mg, 557 μmol, 1.00 eq) in DCM (4 mL) was added TFA (2 mL). The mixture was stirred at 20 °C for 1 h. LC-MS showed the desired mass was detected. The reaction mixture was concentrated under reduced pressure to give the title compound (300 mg, crude) as a black solid. LCMS (ES⁺): m/z 438.4 [M+H]⁺.

Synthesis of 3-(6-(1-(2-(1-(cis-3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-1-methyl-1H-indazol-3-yl)piperidine-2,6-dione (Compound 298). To a solution of 3-(1-methyl-6-(1-(2-(piperidin-4-yl)ethyl)piperidin-4-yl)-1H-indazol-3-yl)piperidine-2,6-dione (100 mg, 228 μmol, 1.00 eq) in DMF (3 mL) was added cis-3-[[5-fluoro-4-[3-(2-oxo-1-pyridyl)phenyl]pyrimidin-2-yl]amino]cyclohexanecarboxylic acid (112 mg, 274 μmol, 1.20 eq), DIEA (59.0 mg, 457 μmol, 79.6 μL, 2.00 eq) and HATU (173 mg, 457 μmol, 2.00 eq) at 20 °C. The mixture was stirred at 20 °C for 2 h. LC-MS showed the desired mass was detected. The reaction mixture was filtered and concentrated under reduced pressure. The residue was purified by prep-HPLC (column: Phenomenex Luna C18 75*30 mm*3 um; mobile phase: [H₂O (0.2% FA)-ACN]; gradient: 10%-50% B over 8.0 min) to give the title compound (Compound 298) (22.2 mg, 25.9 μmol, 11.3% yield, 98.38% purity, 0.300 FA) as a white solid. LCMS (ES⁺): m/z 828.4 [M+H]⁺.

### Example 318. Synthesis of cis-N-((1′-(3-(2,6-dioxopiperidin-3-yl)-2-oxo-2,3-dihydrobenzo[d]oxazol-6-yl)-[1,4′-bipiperidin]-4-yl)methyl)-3-((5-fluoro-4-(3-(2-oxopyridin-1(2H)-yl)phenyl)pyrimidin-2-yl)amino)cyclohexane-1-carboxamide (Compound 299)

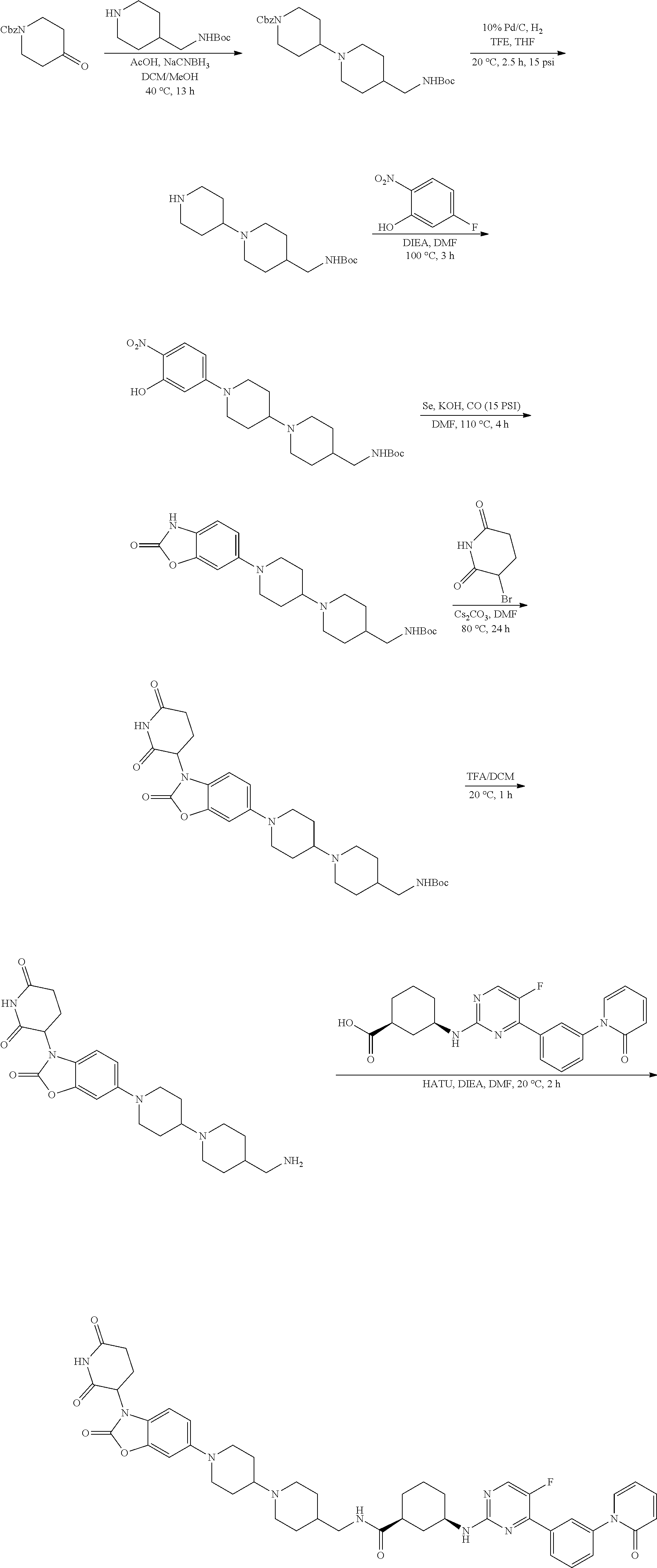

Synthesis of benzyl 4-(((tert-butoxycarbonyl)amino)methyl)-[1,4′-bipiperidine]-1′-carboxylate. Two batches in parallel were set up. To a solution of benzyl 4-oxopiperidine-1-carboxylate (5.00 g, 23.3 mmol, 1.00 eq) in DCM (20 mL) and MeOH (20 mL) was added AcOH (140 mg, 2.33 mmol, 134 μL, 0.100 eq) and tert-butyl (piperidin-4-ylmethyl)carbamate (8.16 g, 35.0 mmol, 6.97 mL, 1.50 eq) at 20 °C and the mixture was stirred at 40 °C for 1 h. Then NaBH₃CN (2.20 g, 35.0 mmol, 1.50 eq) was added to the mixture at 20 °C, the mixture was stirred at 40 °C for 12 h. LCMS showed the desired mass was detected. Two batches in parallel were combined. To the mixture was added H₂O (100 mL) and the mixture was extracted with EtOAc (50 mL*3). The combined organic layers were washed with brine (20 mL*3), dried over Na₂SO₄, filtered and the filtrate was concentrated under reduced pressure to give a residue. The residue was purified by prep-HPLC (column: Waters Xbridge BEH C18 250*70 mm*10 um; mobile phase: [H₂O (0.05% NH₃H₂O)-ACN]; gradient: 37%-67% B over 20.0 min) to give the title compound (1.10 g, 2.22 mmol, 4.75% yield, 87.0% purity) as a white solid. LCMS (ES⁺): m/z 432.3 [M+H]⁺.

Synthesis of tert-butyl ([1,4′-bipiperidin]-4-ylmethyl)carbamate. To a solution of benzyl 4-(((tert-butoxycarbonyl)amino)methyl)-[1,4′-bipiperidine]-1′-carboxylate (1.00 g, 2.32 mmol, 1.00 eq) in TFE (10 mL) and THF (10 mL) was added 10% Pd/C (2.00 g, 50% purity) at 20 °C under N₂. The suspension was degassed under vacuum and purged with H₂ 4 times. The mixture was stirred under H₂ (15 psi) at 20 °C for 2.5 h. LCMS showed benzyl 4-(((tert-butoxycarbonyl)amino)methyl)-[1,4′-bipiperidine]-1′-carboxylate was consumed and the desired mass was detected. The mixture was filtered and the filtrate was concentrated under reduced pressure to give the title compound (960 mg, crude) as a yellow oil. LCMS (ES⁺): m/z 298.2 [M+H]⁺.

Synthesis of tert-butyl ((1′-(3-hydroxy-4-nitrophenyl)-[1,4′-bipiperidin]-4-yl)methyl)carbamate. To a solution of tert-butyl ([1,4′-bipiperidin]-4-ylmethyl)carbamate (960 mg, 3.23 mmol, 1.00 eq) in DMF (20 mL) were added DIEA (1.25 g, 9.68 mmol, 1.69 mL, 3.00 eq) and 5-fluoro-2-nitrophenol (507 mg, 3.23 mmol, 1.00 eq) at 20 °C. The mixture was stirred at 100 °C for 3 h. LCMS showed tert-butyl ([1,4′-bipiperidin]-4-ylmethyl)carbamate was consumed and the desired mass was detected. To the mixture was added H₂O (10 mL) and the mixture was extracted with EtOAc (10 mL*3). The combined organic layers were washed with brine (10 mL*3), dried over Na₂SO₄, filtered and the filtrate was concentrated under reduced pressure to give a residue. The residue was purified by prep-TLC (SiO₂, Dichloromethane/Methanol=5/1) to give the title compound (610 mg, 1.40 mmol, 43.5% yield) as a yellow oil. LCMS (ES⁺): m/z 435.3 [M+H]⁺.

Synthesis of tert-butyl ((1′-(2-oxo-2,3-dihydrobenzo[d]oxazol-6-yl)-[1,4′-bipiperidin]-4-yl)methyl)carbamate. A solution of tert-butyl ((1′-(3-hydroxy-4-nitrophenyl)-[1,4′-bipiperidin]-4-yl)methyl)carbamate (610 mg, 1.40 mmol, 1.00 eq) in DMF (15 mL) were added KOH (158 mg, 2.81 mmol, 2.00 eq) and Se (111 mg, 1.40 mmol, 1.00 eq), pass over CO (15 psi) into the solution. The suspension was degassed and purged with CO for 3 times. The mixture was stirred under CO (15 psi) at 110 °C for 4 h. LCMS showed tert-butyl ((1′-(3-hydroxy-4-nitrophenyl)-[1,4′-bipiperidin]-4-yl)methyl)carbamate was consumed and the desired mass was detected. To the mixture was added H₂O (15 mL) and the mixture was extracted with EtOAc (15 mL*3). The combined organic layers were washed with brine (10 mL*3), dried over Na₂SO₄, filtered and the filtrate was concentrated under reduced pressure to give the title compound (480 mg, crude) as a yellow oil. LCMS (ES⁺): m/z 431.3 [M+H]⁺.

Synthesis of tert-butyl ((1′-(3-(2,6-dioxopiperidin-3-yl)-2-oxo-2,3-dihydrobenzo[d]oxazol-6-yl)-[1,4′-bipiperidin]-4-yl)methyl)carbamate. To a solution of tert-butyl ((1′-(2-oxo-2,3-dihydrobenzo[d]oxazol-6-yl)-[1,4′-bipiperidin]-4-yl)methyl)carbamate (480 mg, 1.11 mmol, 1.00 eq) in DMF (15 mL) was added Cs₂CO₃ (727 mg, 2.23 mmol, 2.00 eq) at 20 °C. Then to the mixture was added 3-bromopiperidine-2,6-dione (428 mg, 2.23 mmol, 2.00 eq) and the mixture was stirred at 80 °C for 12 h. LCMS showed tert-butyl ((1′-(2-oxo-2,3-dihydrobenzo[d]oxazol-6-yl)-[1,4′-bipiperidin]-4-yl)methyl)carbamate remained and the desired mass was detected. Then 3-bromopiperidine-2,6-dione (856 mg, 4.00 eq) was added to the mixture at 20 °C. The mixture was stirred at 80 °C for another 12 h. LCMS showed tert-butyl ((1′-(2-oxo-2,3-dihydrobenzo[d]oxazol-6-yl)-[1,4′-bipiperidin]-4-yl)methyl)carbamate was consumed completely and the desired mass was detected. To the mixture was added H₂O (20 mL) and the mixture was extracted with EtOAc (20 mL*3). The combined organic layers were washed with brine (10 mL*3), dried over Na₂SO₄, filtered and the filtrate was concentrated under reduced pressure to give a residue. The residue was purified by prep-TLC (SiO₂, PE/THF=1/1) to give the title compound (120 mg, 222 μmol, 19.9% yield) as a white solid. LCMS (ES⁺): m/z 542.4 [M+H]⁺.

Synthesis of 3-(6-(4-(aminomethyl)-[1,4′-bipiperidin]-1′-yl)-2-oxobenzo[d]oxazol-3 (2H)-yl)piperidine-2,6-dione. A solution of tert-butyl ((1′-(3-(2,6-dioxopiperidin-3-yl)-2-oxo-2,3-dihydrobenzo[d]oxazol-6-yl)-[1,4′-bipiperidin]-4-yl)methyl)carbamate (100 mg, 185 mol, 1.00 eq) in DCM (2 mL) and TFA (0.4 mL) was stirred at 20 °C for 1 h. LCMS showed tert-butyl ((1′-(3-(2,6-dioxopiperidin-3-yl)-2-oxo-2,3-dihydrobenzo[d]oxazol-6-yl)-[1,4′-bipiperidin]-4-yl)methyl)carbamate was consumed and the desired mass was detected. The mixture was concentrated under reduced pressure to give the title compound (100 mg, crude, TFA) as a yellow oil. LCMS (ES⁺): m/z 442.3 [M+H]⁺.

Synthesis of cis-N-((1′-(3-(2,6-dioxopiperidin-3-yl)-2-oxo-2,3-dihydrobenzo[d]oxazol-6-yl)-[1,4′-bipiperidin]-4-yl)methyl)-3-((5-fluoro-4-(3-(2-oxopyridin-1(2H)-yl)phenyl)pyrimidin-2-yl)amino)cyclohexane-1-carboxamide (Compound 299). To a solution of cis-3-((5-fluoro-4-(3-(2-oxopyridin-1(2H)-yl)phenyl)pyrimidin-2-yl)amino)cyclohexane-1-carboxylic acid (36.8 mg, 90.0 μmol, 0.500 eq) in DMF (2 mL) were added HATU (103 mg, 270 μmol, 1.50 eq) and DIEA (69.8 mg, 540 μmol, 94.1 μL, 3.00 eq) at 20 °C, then 3-(6-(4-(aminomethyl)-[1,4′-bipiperidin]-1′-yl)-2-oxobenzo[d]oxazol-3 (2H)-yl)piperidine-2,6-dione (100 mg, 180 μmol, 1.00 eq, TFA) was added to the mixture at 20 °C. The mixture was stirred at 20 °C for 2 h. LCMS showed 3-(6-(4-(aminomethyl)-[1,4′-bipiperidin]-1′-yl)-2-oxobenzo[d]oxazol-3 (2H)-yl)piperidine-2,6-dione was consumed and the desired mass was detected. To the mixture was added H₂O (5 mL) and the mixture was extracted with EtOAc (5 mL*3). The combined organic layers were washed with brine (5 mL*3), dried over Na₂SO₄, filtered and the filtrate was concentrated under reduced pressure to give a residue. The residue was purified by prep-HPLC (column: Phenomenex Luna C18 100*30 mm*3 um; mobile phase: [H₂O (0.2% FA)-ACN]; gradient: 5%-35% B over 8.0 min) to give the title compound (Compound 299) (14.3 mg, 15.5 μmol, 8.59% yield, 90.0% purity) as a white solid. LCMS (ES⁺): m/z 832.4 [M+H]⁺

### Example 319. Synthesis of cis-N-((1′-(4-((2,6-dioxopiperidin-3-yl)amino)phenyl)-[1,4′-bipiperidin]-4-yl)methyl)-3-((5-fluoro-4-(3-(2-oxopyridin-1(2H)-yl)phenyl)pyrimidin-2-yl)amino)cyclohexane-1-carboxamide (Compound 297)

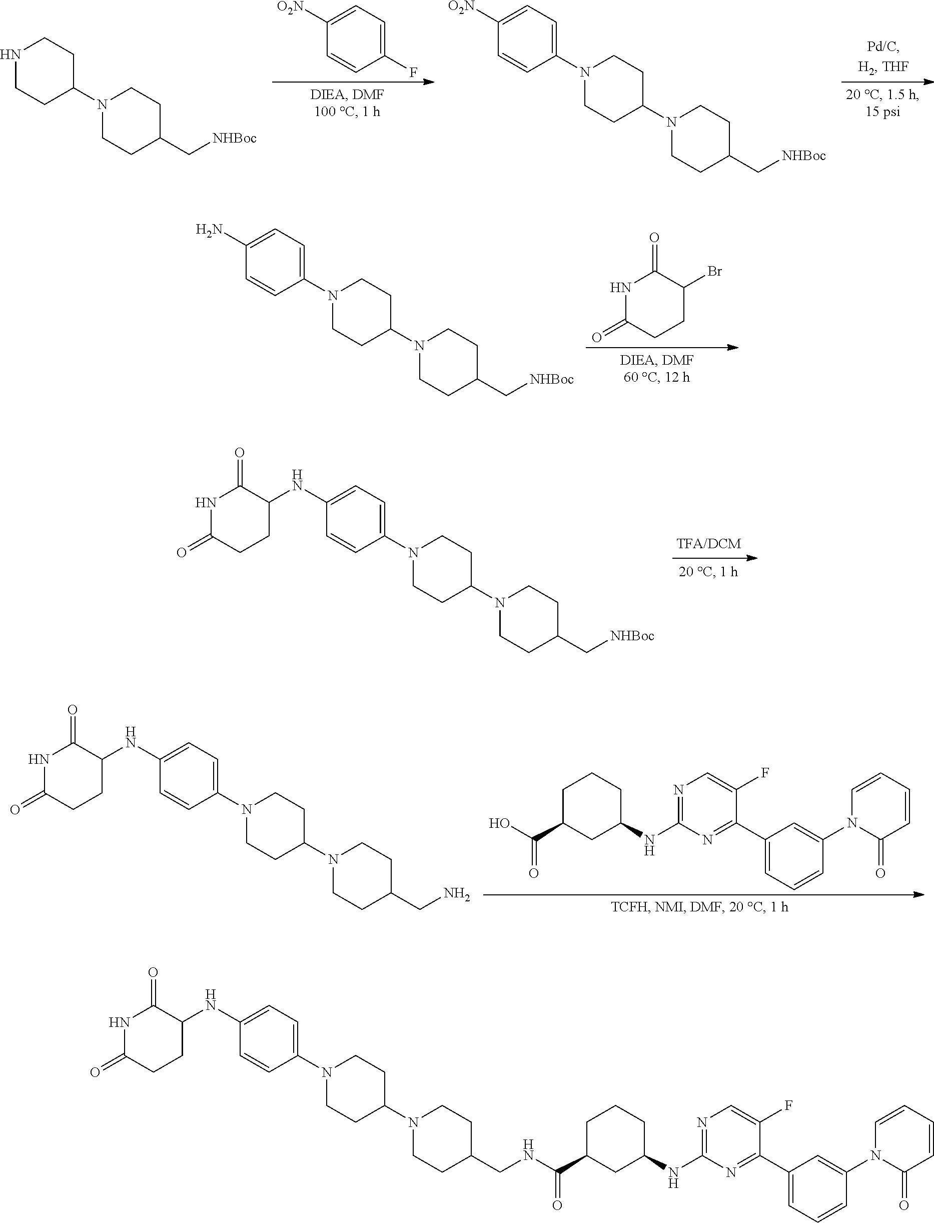

Synthesis of tert-butyl ((1′-(4-nitrophenyl)-[1,4′-bipiperidin]-4-yl)methyl)carbamate. To a solution of tert-butyl ([1,4′-bipiperidin]-4-ylmethyl)carbamate (360 mg, 1.21 mmol, 1.00 eq) in DMF (1 mL) were added DIEA (469 mg, 3.63 mmol, 632 μL, 3.00 eq) and 1-fluoro-4-nitrobenzene (171 mg, 1.21 mmol, 128 μL, 1.00 eq) at 20 °C. The mixture was stirred at 100 °C for 1 h. LCMS showed tert-butyl ([1,4′-bipiperidin]-4-ylmethyl)carbamate was consumed completely and the desired mass was detected. H₂O (5 ml) was added to the mixture, then the mixture was filtered, the filter cake was dried under reduced pressure to give the title compound (200 mg, crude) as a yellow solid. LCMS (ES+): m/z 419.3 [M+H]⁺.

Synthesis of tert-butyl ((1′-(4-aminophenyl)-[1,4′-bipiperidin]-4-yl)methyl)carbamate. To a solution of tert-butyl ((1′-(4-nitrophenyl)-[1,4′-bipiperidin]-4-yl)methyl)carbamate (170 mg, 406 μmol, 1.00 eq) in THE (2 mL) was added 10% Pd/C (100 mg, 50% purity) at 20 °C under N₂. The suspension was degassed under vacuum and purged with H₂ 4 times. The mixture was stirred under H₂ (15 psi) at 20 °C for 1.5 h. LCMS showed tert-butyl ((1′-(4-nitrophenyl)-[1,4′-bipiperidin]-4-yl)methyl)carbamate was consumed completely and the desired mass was detected. The mixture was filtered and the filtrate was concentrated under reduced pressure to give the title compound (120 mg, crude) as a yellow oil. LCMS (ES⁺): m/z 389.3 [M+H]⁺.

Synthesis of tert-butyl ((1′-(4-((2,6-dioxopiperidin-3-yl)amino)phenyl)-[1,4′-bipiperidin]-4-yl)methyl)carbamate. To a solution of tert-butyl ((1′-(4-aminophenyl)-[1,4′-bipiperidin]-4-yl)methyl)carbamate (120 mg, 309 μmol, 1.00 eq) and 3-bromopiperidine-2,6-dione (148 mg, 772 μmol, 2.50 eq) in DMF (1.5 mL) was added DIEA (120 mg, 927 μmol, 161 μL, 3.00 eq) at 20 °C. The mixture was stirred at 60 °C for 12 h. LCMS showed tert-butyl ((1′-(4-aminophenyl)-[1,4′-bipiperidin]-4-yl)methyl)carbamate was consumed completely and the desired mass was detected. The mixture was diluted with H₂O (8 mL), the mixture was filtered and the filter cake was dried under reduced pressure to give the title compound (100 mg, crude) as a brown solid. LCMS (ES⁺): m/z 500.4 [M+H]⁺.

Synthesis of 3-((4-(4-(aminomethyl)-[1,4′-bipiperidin]-1′-yl)phenyl)amino)piperidine-2,6-dione. A solution of tert-butyl ((1′-(4-((2,6-dioxopiperidin-3-yl)amino)phenyl)-[1,4′-bipiperidin]-4-yl)methyl)carbamate (70.0 mg, 140 μmol, 1.00 eq) in DCM (0.6 mL) and TFA (0.2 mL) was stirred at 20 °C for 1 h. LCMS showed tert-butyl ((1′-(4-((2,6-dioxopiperidin-3-yl)amino)phenyl)-[1,4′-bipiperidin]-4-yl)methyl)carbamate was consumed and the desired mass was detected. The reaction mixture was concentrated under reduced pressure to give the title compound (70.0 mg, crude, TFA) as a brown oil. LCMS (ES⁺): m/z 400.3 [M+H]⁺.

Synthesis of cis-N-((1′-(4-((2,6-dioxopiperidin-3-yl)amino)phenyl)-[1,4′-bipiperidin]-4-yl)methyl)-3-((5-fluoro-4-(3-(2-oxopyridin-1(2H)-yl)phenyl)pyrimidin-2-yl)amino)cyclohexane-1-carboxamide (Compound 297). To a solution of cis-3-((5-fluoro-4-(3-(2-oxopyridin-1(2H)-yl)phenyl)pyrimidin-2-yl)amino)cyclohexane-1-carboxylic acid (27.8 mg, 68.2 μmol, 0.500 eq) in DMF (0.2 mL) were added TCFH (45.9 mg, 164 μmol, 1.20 eq) and NMI (67.2 mg, 818 μmol, 65.2 μL, 6.00 eq) at 20 °C, then 3-((4-(4-(aminomethyl)-[1,4′-bipiperidin]-1′-yl)phenyl)amino)piperidine-2,6-dione (70.0 mg, 136 μmol, 1.00 eq, TFA) was added to the mixture, the mixture was stirred at 20 °C for 1 h. LCMS showed 3-((4-(4-(aminomethyl)-[1,4′-bipiperidin]-1′-yl)phenyl)amino)piperidine-2,6-dione was consumed and the desired mass was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 5%-45% B over 8.0 min) to give the title compound (Compound 297) (2.60 mg, 3.11 μmol, 2.28% yield, 100% purity, FA) as a black solid. LCMS (ES⁺): m/z 790.4 [M+H]⁺

### Example 320. Synthesis of cis-N-(1-((1-(4-((2,6-dioxopiperidin-3-yl)amino)-2-fluorophenyl)piperidin-4-yl)methyl)piperidin-4-yl)-3-((5-fluoro-4-(3-(2-oxopyridin-1(2H)-yl)phenyl)pyrimidin-2-yl)amino)cyclohexane-1-carboxamide (Compound 336)

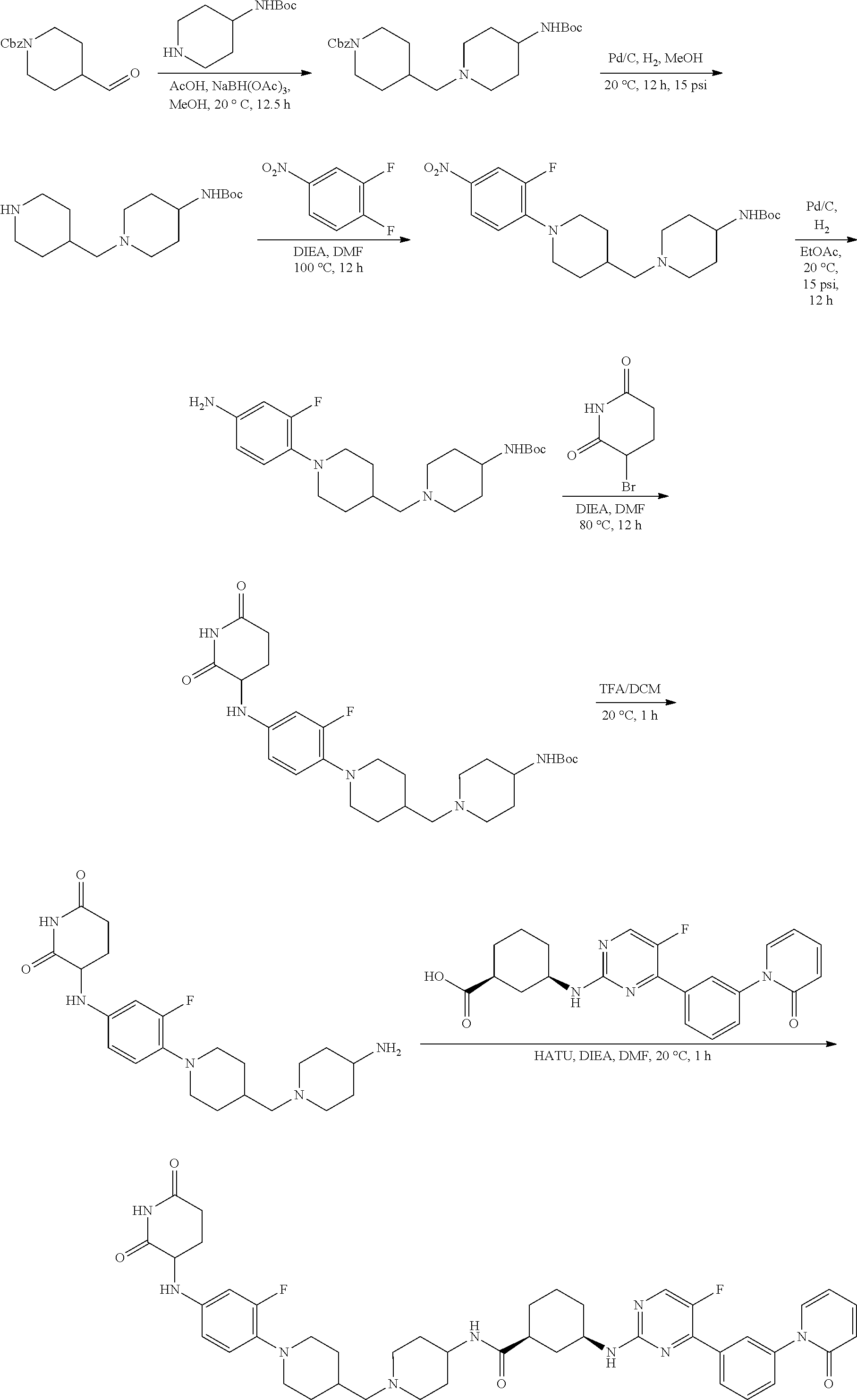

Synthesis of benzyl 4-((4-((tert-butoxycarbonyl)amino)piperidin-1-yl)methyl)piperidine-1-carboxylate. To a solution of benzyl 4-formylpiperidine-1-carboxylate (5.00 g, 20.2 mmol, 1.00 eq) and tert-butyl N-(4-piperidyl)carbamate (4.86 g, 24.3 mmol, 1.20 eq) in MeOH (100 mL) was added AcOH (607 mg, 10.1 mmol, 579 μL, 0.500 eq) at 20 °C, the mixture was stirred at 20 °C for 0.5 h, then NaBH(OAc)₃ (4.29 g, 20.2 mmol, 1.00 eq) was added to the mixture at 20 °C. The mixture was stirred at 20 °C for 12 h. LC-MS showed that 4-formylpiperidine-1-carboxylate was consumed completely and desired mass was detected. The reaction mixture was concentrated under reduced pressure to give a residue. The residue was purified by column chromatography (SiO₂, Petroleum ether/Ethyl acetate=1/0 to 0/1) to give compound the title (8.00 g, crude) as a white solid. LCMS (ES⁺): m/z 432.2 [M+H]⁺.

Synthesis of tert-butyl (1-(piperidin-4-ylmethyl)piperidin-4-yl)carbamate. To a mixture of 10% Pd/C (4 g, 50% purity) in MeOH (100 mL) was added benzyl 4-((4-((tert-butoxycarbonyl)amino)piperidin-1-yl)methyl)piperidine-1-carboxylate (8.00 g, 18.5 mmol, 1.00 eq) at 20 °C under N₂. The mixture was purged and degassed with H₂ for 3 times. The final mixture was stirred at 20 °C for 12 h under H₂ (15 psi). LC-MS showed that benzyl 4-((4-((tert-butoxycarbonyl)amino)piperidin-1-yl)methyl)piperidine-1-carboxylate was consumed completely and desired mass was detected. The mixture was filtered and filtrate was concentrated under reduced pressure to give the title compound (5.80 g, crude) as a white solid. LCMS (ES⁺): m/z 298.2 [M+H]⁺.

Synthesis of tert-butyl (1-((1-(2-fluoro-4-nitrophenyl)piperidin-4-yl)methyl)piperidin-4-yl)carbamate. To a solution of tert-butyl (1-(piperidin-4-ylmethyl)piperidin-4-yl)carbamate (5.80 g, 19.5 mmol, 1.00 eq) and 1,2-difluoro-4-nitro-benzene (3.72 g, 23.4 mmol, 2.59 mL, 1.20 eq) in DMF (80 mL) was added DIEA (6.30 g, 48.8 mmol, 8.49 mL, 2.50 eq) at 20 °C. The mixture was stirred at 100 °C for 12 h. LC-MS showed that tert-butyl (1-(piperidin-4-ylmethyl)piperidin-4-yl)carbamate was consumed completely and the desired mass was detected. The residue was diluted with H₂O (50 mL) and extracted with EtOAc (50 mL*3). The combined organic layers were washed with brine (50 mL), dried over Na₂SO₄, filtered and the filtrate was concentrated under reduced pressure to give a residue. The residue was purified by column chromatography (SiO₂, Petroleum ether/Ethyl acetate=1/0 to 0/1) to give the title compound (7.50 g, crude) as a yellow solid. LCMS (ES⁺): m/z 437.2 [M+H]⁺.

Synthesis of tert-butyl (1-((1-(4-amino-2-fluorophenyl)piperidin-4-yl)methyl)piperidin-4-yl)carbamate. To a mixture of 10% Pd/C (2.00 g, 50% purity) in EtOAc (50 mL) was added tert-butyl (1-((1-(2-fluoro-4-nitrophenyl)piperidin-4-yl)methyl)piperidin-4-yl)carbamate (7.50 g, 17.2 mmol, 1.00 eq) at 20 °C under N₂. The mixture was purged and degassed with H₂ for 3 times. The final mixture was stirred at 20 °C for 12 h under H₂ (15 psi). LC-MS showed tert-butyl (1-((1-(2-fluoro-4-nitrophenyl)piperidin-4-yl)methyl)piperidin-4-yl)carbamate was consumed completely and the desired mass was detected. The mixture was filtered and the filtrate was concentrated under reduced pressure to give the title compound (5.80 g, crude) as a white solid. LCMS (ES⁺): m/z 407.2 [M+H]⁺.

Synthesis of tert-butyl (1-((1-(4-((2,6-dioxopiperidin-3-yl)amino)-2-fluorophenyl)piperidin-4-yl)methyl)piperidin-4-yl)carbamate. To a solution of tert-butyl (1-((1-(4-amino-2-fluorophenyl)piperidin-4-yl)methyl)piperidin-4-yl)carbamate (1.00 g, 2.46 mmol, 1.00 eq) and 3-bromopiperidine-2,6-dione (1.89 g, 9.84 mmol, 4.00 eq) in DMF (20 mL) was added DIEA (954 mg, 7.38 mmol, 1.29 mL, 3.00 eq) at 20 °C. The mixture was stirred at 80 °C for 12 h. LC-MS showed that tert-butyl (1-((1-(4-amino-2-fluorophenyl)piperidin-4-yl)methyl)piperidin-4-yl)carbamate was consumed completely and desired mass was detected. The residue was diluted with H₂O (20 mL) and extracted with EtOAc (20 mL*3). The combined organic layers were washed with brine (20 mL), dried over Na₂SO₄, filtered and the filtrate was concentrated under reduced pressure to give a residue. The residue was purified by prep-HPLC (column: Phenomenex luna C18 (250*70 mm, 15 um); mobile phase: [H₂O (0.2% FA)-ACN]; gradient: 10%-40% B over 20.0 min) to give the title compound (200 mg, crude) as a green solid. LCMS (ES⁺): m/z 518.3 [M+H]⁺.

Synthesis of 3-((4-(4-((4-aminopiperidin-1-yl)methyl)piperidin-1-yl)-3-fluorophenyl)amino)piperidine-2,6-dione. To a solution of tert-butyl (1-((1-(4-((2,6-dioxopiperidin-3-yl)amino)-2-fluorophenyl)piperidin-4-yl)methyl)piperidin-4-yl)carbamate (100 mg, 193 μmol, 1.00 eq) in DCM (1.5 mL) was added TFA (0.5 mL) at 20 °C. The mixture was stirred at 20 °C for 1 h. LC-MS showed that tert-butyl (1-((1-(4-((2,6-dioxopiperidin-3-yl)amino)-2-fluorophenyl)piperidin-4-yl)methyl)piperidin-4-yl)carbamate was consumed completely and the desired mass was detected. The reaction mixture was concentrated under reduced pressure to give the title compound (80 mg, crude, TFA) as a brown oil. LCMS (ES⁺): m/z 418.2 [M+H]⁺.

Synthesis of cis-N-(1-((1-(4-((2,6-dioxopiperidin-3-yl)amino)-2-fluorophenyl)piperidin-4-yl)methyl)piperidin-4-yl)-3-((5-fluoro-4-(3-(2-oxopyridin-1(2H)-yl)phenyl)pyrimidin-2-yl)amino)cyclohexane-1-carboxamide (Compound 336). To a solution of 3-((4-(4-((4-aminopiperidin-1-yl)methyl)piperidin-1-yl)-3-fluorophenyl)amino)piperidine-2,6-dione (80 mg, 191.6 μmol, 1.00 eq) and cis-3-((5-fluoro-4-(3-(2-oxopyridin-1(2H)-yl)phenyl)pyrimidin-2-yl)amino)cyclohexane-1-carboxylic acid (78.3 mg, 191.6 μmol, 1.00 eq) in DMF (3 mL) were added HATU (146 mg, 383 μmol, 2.00 eq) and DIEA (74.3 mg, 575 mol, 100 μL, 3.00 eq) at 20 °C. The mixture was stirred at 20 °C for 1 h. LC-MS showed that 3-((4-(4-((4-aminopiperidin-1-yl)methyl)piperidin-1-yl)-3-fluorophenyl)amino)piperidine-2,6-dione was consumed completely and the desired mass was detected. The residue was diluted with H₂O (5 mL) and extracted with EtOAc (3 mL*3). The combined organic layers were washed with brine (4 mL), dried over Na₂SO₄, filtered and concentrated under reduced pressure to give a residue. The residue was purified by prep-HPLC (column: Phenomenex luna C18 100*40 mm*5 um; mobile phase: [H₂O (0.2% FA)-ACN]; gradient: 1%-40% B over 8.0 min) to give the title compound (Compound 336) (13 mg, 15.6 μmol, 8.12% yield, 99.4% purity, 0.5 FA) as a white solid. LCMS (ES⁺): m/z 808.5 [M+H]⁺.

### Example 321. Synthesis of trans-N-(cis-4-((4-(4-((2,6-dioxopiperidin-3-yl)amino)-2-fluorophenyl)piperidin-1-yl)methyl)cyclohexyl)-3-((5-fluoro-4-(3-(2-oxopyridin-1(2H)-yl)phenyl)pyrimidin-2-yl)amino)cyclohexane-1-carboxamide (Compound 324)

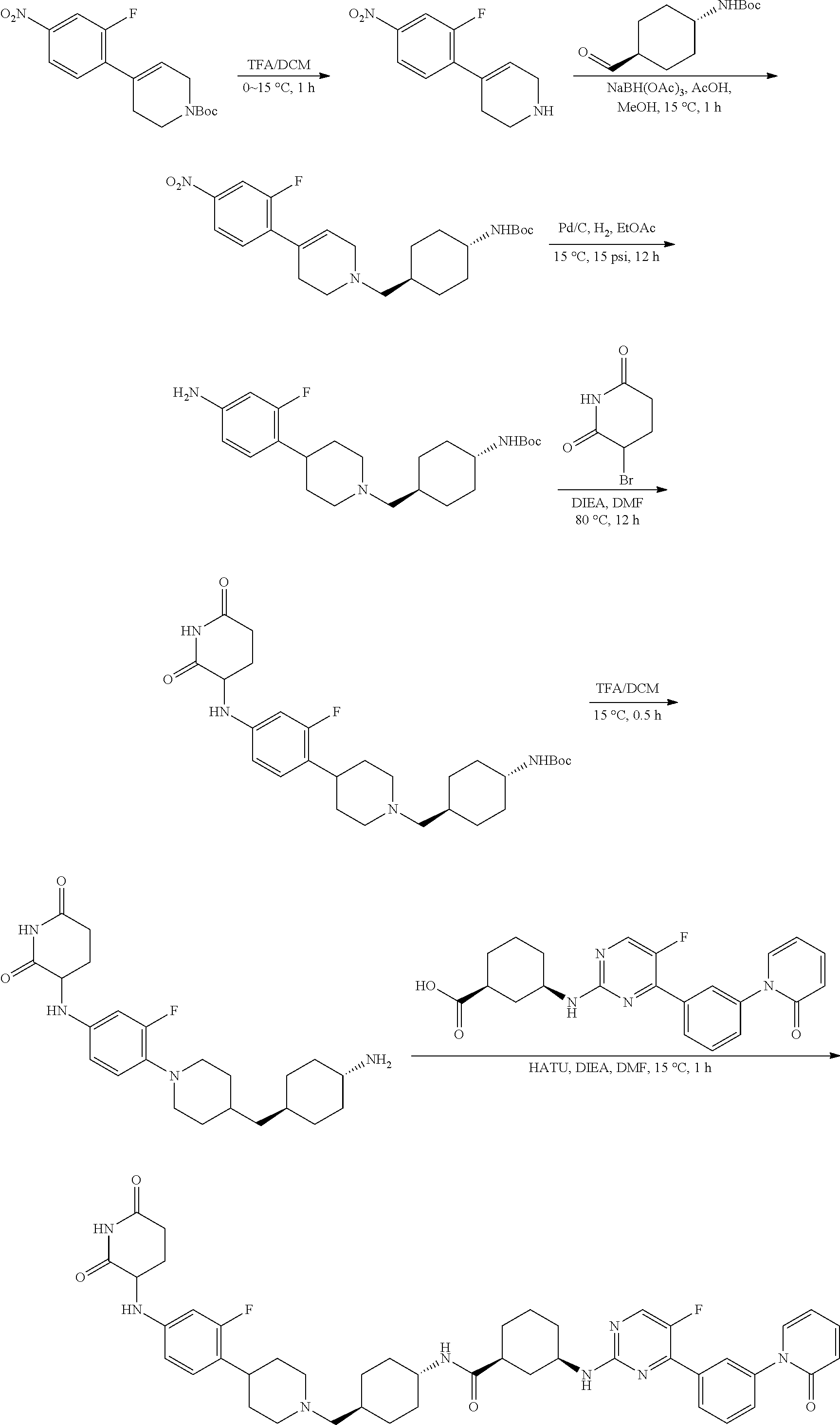

Synthesis of 4-(2-fluoro-4-nitrophenyl)-1,2,3,6-tetrahydropyridine. To a solution of tert-butyl 4-(2-fluoro-4-nitrophenyl)-3,6-dihydropyridine-1(2H)-carboxylate (7.00 g, 21.7 mmol, 1.00 eq) in DCM (10.0 mL) was added TFA (15.3 g, 134 mmol, 10.0 mL, 6.20 eq) at 0 °C. The mixture was stirred at 15 °C for 1 h. LC-MS showed that tert-butyl 4-(2-fluoro-4-nitrophenyl)-3,6-dihydropyridine-1(2H)-carboxylate was consumed completely and the desired mass was detected. The mixture was concentrated under reduced pressure to give title compound (12.0 g, crude, TFA) as a brown solid. LCMS (ES⁺): m/z 223.1 [M+H]⁺.

Synthesis of trans-tert-butyl (4-((4-(2-fluoro-4-nitrophenyl)-3,6-dihydropyridin-1(2H)-yl)methyl)cyclohexyl)carbamate. To a solution of 4-(2-fluoro-4-nitrophenyl)-1,2,3,6-tetrahydropyridine (2.00 g, 5.95 mmol, 1.00 eq, TFA) in MeOH (10.0 mL) was added trans-tert-butyl (4-formylcyclohexyl)carbamate (2.70 g, 11.9 mmol, 2.00 eq), AcOH (714 mg, 11.9 mmol, 681 μL, 2.00 eq) and NaBH(OAc)₃ (2.52 g, 11.9 mmol, 2.00 eq) at 15 °C. The mixture was stirred at 15 °C for 1 h. LC-MS showed that 4-(2-fluoro-4-nitrophenyl)-1,2,3,6-tetrahydropyridine was consumed completely and the desired mass was detected. The mixture was concentrated under reduced pressure to give a residue, the reaction mixture was added water (20 mL) and extracted with DCM (20 mL*3). The combined organic layers were washed with brine (20 mL*3), dried over Na₂SO₄, filtered and the filtrate was concentrated under reduced pressure to give a residue. The residue was purified by column chromatography (SiO₂, Petroleum ether/Ethyl acetate=3/1 to 1/1) to give title compound (800 mg, 1.85 mmol, 31.0% yield) as a yellow oil. LCMS (ES⁺): m/z 434.2 [M+H]⁺.

Synthesis of trans-tert-butyl (4-((4-(4-amino-2-fluorophenyl)piperidin-1-yl)methyl)cyclohexyl)carbamate. To a solution of trans-tert-butyl (4-((4-(2-fluoro-4-nitrophenyl)-3,6-dihydropyridin-1(2H)-yl)methyl)cyclohexyl)carbamate (800 mg, 1.85 mmol, 1.00 eq) in EtOAc (2 mL) was added 10% Pd/C (589 mg, 2.77 mmol, 50.0% purity, 1.50 eq) at 15 °C. The mixture was stirred at 15 °C for 12 h under H₂ (15 psi). LC-MS showed that trans-tert-butyl (4-((4-(2-fluoro-4-nitrophenyl)-3,6-dihydropyridin-1(2H)-yl)methyl)cyclohexyl)carbamate was consumed completely and the desired mass was detected. The reaction mixture was filtered and the filterate was concentrated under reduced pressure to give a residue to give title compound (500 mg, crude) as a white solid. LCMS (ES⁺): m/z 406.3 [M+H]⁺.

Synthesis of cis-tert-butyl (4-((4-(4-((2,6-dioxopiperidin-3-yl)amino)-2-fluorophenyl)piperidin-1-yl)methyl)cyclohexyl)carbamate. To a solution of cis-trans-tert-butyl (4-((4-(4-amino-2-fluorophenyl)piperidin-1-yl)methyl)cyclohexyl)carbamate (400 mg, 986 μmol, 1.00 eq) in DMF (2.00 mL) was added DIPEA (382 mg, 2.96 mmol, 515 μL, 3.00 eq) and 3-bromopiperidine-2,6-dione (757 mg, 3.95 mmol, 4.00 eq) at 15 °C. The mixture was stirred at 80 °C for 12 h. LC-MS showed that trans-tert-butyl (4-((4-(4-amino-2-fluorophenyl)piperidin-1-yl)methyl)cyclohexyl)carbamate was consumed completely and the desired mass was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 5%-40% B over 8.0 min) to give title compound (220 mg, 425 μmol, 43.1% yield) as a yellow solid. LCMS (ES⁺): m/z 517.6 [M+H]⁺.

Synthesis of cis-3-((4-(4-((4-aminocyclohexyl)methyl)piperidin-1-yl)-3-fluorophenyl)amino)piperidine-2,6-dione. To a solution of trans-tert-butyl (4-((4-(4-((2,6-dioxopiperidin-3-yl)amino)-2-fluorophenyl)piperidin-1-yl)methyl)cyclohexyl)carbamate (90.0 mg, 174 μmol, 1.00 eq) in DCM (0.50 mL) was added TFA (2.07 g, 18.1 mmol, 1.35 mL, 104 eq) at 0 °C. The mixture was stirred at 15 °C for 0.5 h. LC-MS showed that trans-tert-butyl (4-((4-(4-((2,6-dioxopiperidin-3-yl)amino)-2-fluorophenyl)piperidin-1-yl)methyl)cyclohexyl)carbamate was consumed completely and the desired mass was detected. The mixture was concentrated under reduced pressure to give the title compound (60 mg, crude, TFA) as a yellow oil. LCMS (ES⁺): m/z 417.5 [M+H]⁺.

Synthesis of trans-N-(cis-4-((4-(4-((2,6-dioxopiperidin-3-yl)amino)-2-fluorophenyl)piperidin-1-yl)methyl)cyclohexyl)-3-((5-fluoro-4-(3-(2-oxopyridin-1(2H)-yl)phenyl)pyrimidin-2-yl)amino)cyclohexane-1-carboxamide (Compound 324). To a solution of cis-3-((4-(4-((4-aminocyclohexyl)methyl)piperidin-1-yl)-3-fluorophenyl)amino)piperidine-2,6-dione (55.0 mg, 103 μmol, 1.00 eq, TFA) in DMF (2.00 mL) were added DIPEA (40.1 mg, 311 μmol, 54.1 μL, 3.00 eq), cis-(1R,3S)-3-((5-fluoro-4-(3-(2-oxopyridin-1(2H)-yl)phenyl)pyrimidin-2-yl)amino)cyclohexane-1-carboxylic acid (46.5 mg, 114 μmol, 1.10 eq) and HATU (59.1 mg, 155 μmol, 1.50 eq) at 15 °C. The mixture was stirred at 15 °C for 1 h. LC-MS showed that cis-3-((4-(4-((4-aminocyclohexyl)methyl)piperidin-1-yl)-3-fluorophenyl)amino)piperidine-2,6-dione was consumed completely and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-70% B over 8.0 min) to give title Compound (Compound 324) (3.10 mg, 3.58 μmol, 3.45% yield, 93.1% purity) as a yellow solid. LCMS (ES⁺): m/z 807.5 [M+H]⁺.

### Example 322. Synthesis of cis-N-((1′-(4-((2,6-dioxopiperidin-3-yl)amino)-2-fluorophenyl)-[1,4′-bipiperidin]-4-yl)methyl)-3-((5-fluoro-4-(3-(2-oxopyridin-1(2H)-yl)phenyl)pyrimidin-2-yl)amino)cyclohexane-1-carboxamide (Compound 361)

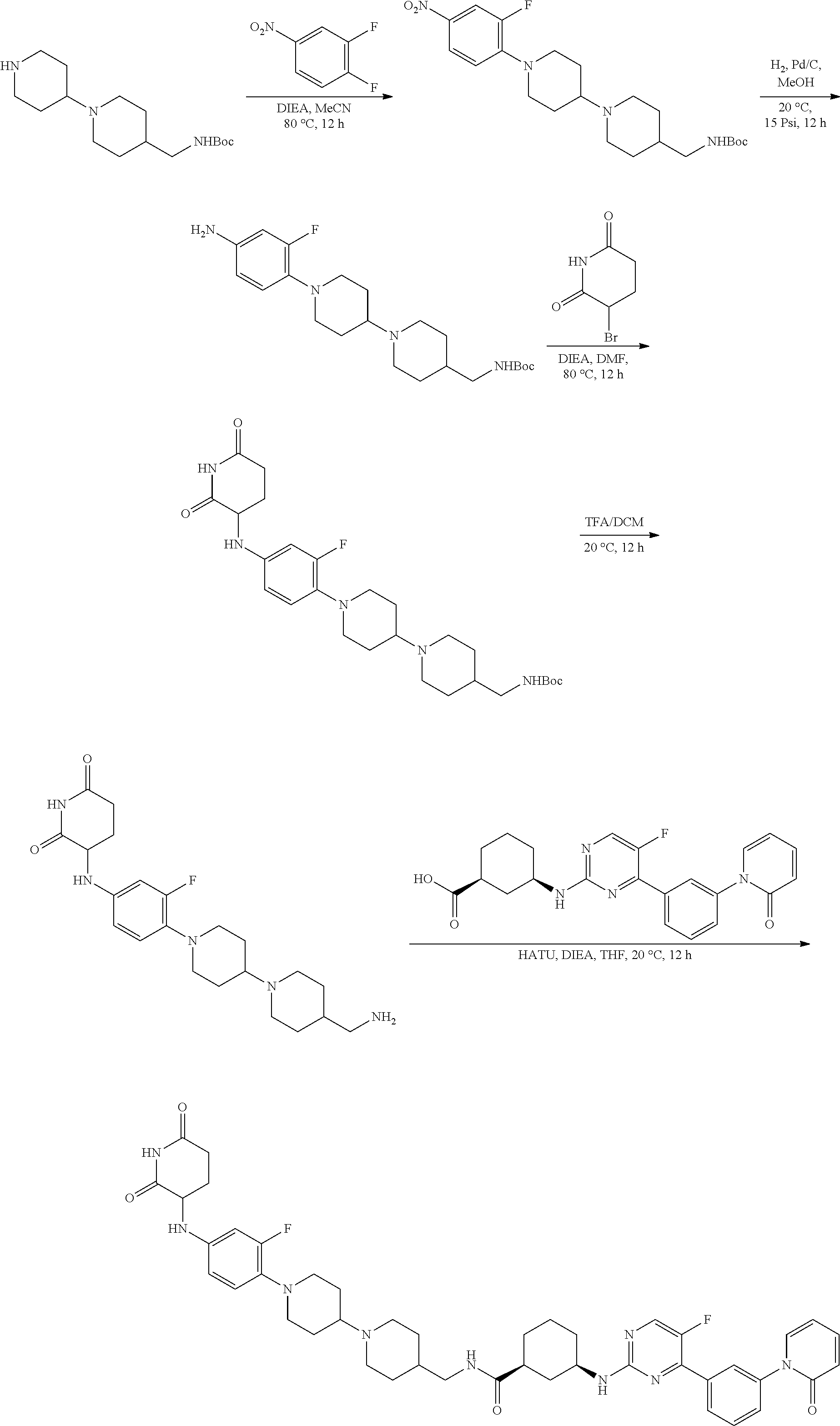

Synthesis of tert-butyl ((1′-(2-fluoro-4-nitrophenyl)-[1,4′-bipiperidin]-4-yl)methyl)carbamate. To a solution of benzyl 4-(((tert-butoxycarbonyl)amino)methyl)-[1,4′-bipiperidine]-1′-carboxylate (3.80 g, 12.8 mmol, 1.00 eq) in ACN (40.0 mL) were added 1,2-difluoro-4-nitrobenzene (2.43 g, 15.3 mmol, 1.69 mL, 1.20 eq) and DIEA (3.30 g, 25.6 mmol, 4.45 mL, 2.00 eq) at 20 °C. The mixture was stirred at 80 °C for 12 h. LC-MS showed that the desired mass was detected. The mixture was concentrated under reduced pressure. The residue was purified by column chromatography (SiO₂, Petroleum ether:Ethyl acetate=1:0 to 0:1) to give the title compound (4.50 g, 10.3 mmol, 80.7% yield, n/a purity) as a yellow oil. LCMS (ES⁺): m/z 437.2 [M+H]⁺.

Synthesis of tert-butyl ((1′-(4-amino-2-fluorophenyl)-[1,4′-bipiperidin]-4-yl)methyl)carbamate. To a mixture of 10% Pd/C (1.00 g, 50.0% purity) in MeOH (50.0 mL) was added tert-butyl ((1′-(2-fluoro-4-nitrophenyl)-[1,4′-bipiperidin]-4-yl)methyl)carbamate (4.50 g, 10.3 mmol, 1.00 eq) at 20 °C under N₂ atmosphere. The mixture was stirred at 20 °C for 12 h under H₂ (15 Psi). LC-MS showed that the desired mass was detected. The mixture was filtered and the filtrate was concentrated under reduced pressure to give the title compound (3.50 g, crude) as a brown solid. LCMS (ES⁺): m/z 407.3 [M+H]⁺.

Synthesis of tert-butyl ((1′-(4-((2,6-dioxopiperidin-3-yl)amino)-2-fluorophenyl)-[1,4′-bipiperidin]-4-yl)methyl)carbamate. To a solution of tert-butyl ((1′-(4-amino-2-fluorophenyl)-[1,4′-bipiperidin]-4-yl)methyl)carbamate (3.50 g, 8.61 mmol, 1.00 eq) in DMF (15.0 mL) were added 3-bromopiperidine-2,6-dione (4.96 g, 25.8 mmol, 3.00 eq) and DIEA (2.22 g, 17.2 mmol, 2.99 mL, 2.00 eq) at 20 °C. The mixture was stirred at 80 °C for 12 h. LC-MS showed that the desired mass was detected. The mixture was filtered and the filtrate was concentrated under reduced pressure. The residue was purified by prep-HPLC (column: Phenomenex luna c18 250 mm*100 mm*10 um; mobile phase: [H₂O (0.2% FA)-ACN]; gradient: 2%-25% B over 20.0 min) to give the title compound (500 mg, 966 μmol, 11.2% yield, n/a purity) as a purple solid. LCMS (ES⁺): m/z 518.3 [M+H]⁺.

Synthesis of 3-((4-(4-(aminomethyl)-[1,4′-bipiperidin]-1′-yl)-3-fluorophenyl)amino)piperidine-2,6-dione. To a solution of tert-butyl ((1′-(4-((2,6-dioxopiperidin-3-yl)amino)-2-fluorophenyl)-[1,4′-bipiperidin]-4-yl)methyl)carbamate (500 mg, 966 μmol, 1.00 eq) in DCM (10.0 mL) was added TFA (3.84 g, 33.7 mmol, 2.50 mL, 34.8 eq) at 20 °C. The mixture was stirred at 20 °C for 12 h. LC-MS showed that the desired mass was detected. The mixture was concentrated under reduced pressure to give the title compound (500 mg, crude, TFA) as a brown oil. LCMS (ES⁺): m/z 418.2 [M+H]⁺.

Synthesis of Synthesis of cis-N-((1′-(4-((2,6-dioxopiperidin-3-yl)amino)-2-fluorophenyl)-[1,4′-bipiperidin]-4-yl)methyl)-3-((5-fluoro-4-(3-(2-oxopyridin-1(2H)-yl)phenyl)pyrimidin-2-yl)amino)cyclohexane-1-carboxamide (Compound 361). The reaction was set up in 5 parallel batches. To a solution of 3-((4-(4-(aminomethyl)-[1,4′-bipiperidin]-1′-yl)-3-fluorophenyl)amino)piperidine-2,6-dione (20.0 mg, 37.6 μmol, 1.00 eq, TFA), cis-3-((5-fluoro-4-(3-(2-oxopyridin-1(2H)-yl)phenyl)pyrimidin-2-yl)amino)cyclohexane-1-carboxylic acid (18.4 mg, 45.1 μmol, 1.20 eq) in THF (1 mL) were added DIEA (24.3 mg, 188 μmol, 32.8 μL, 5.00 eq) and HATU (42.9 mg, 113 μmol, 3.00 eq) at 20 °C. The mixture was stirred at 20 °C for 12 h. LC-MS showed the desired mass was detected. 5 batches were combined to work up. The mixture was concentrated under reduced pressure. The residue was purified by prep-HPLC (column: Phenomenex luna C18 100*40 mm*3 um; mobile phase: [H₂O (0.2% FA)-ACN]; gradient: 10%-50% B over 8.0 min) to give the title compound (Compound 361) (40.2 mg, 47.0 μmol, 25.0% yield, 96.0% purity, 0.3 FA) as a gray solid. LCMS (ES⁺): m/z 808.3 [M+H]⁺.

### Example 323. Synthesis of cis-N-((cis-4-(4-(4-((2,6-dioxopiperidin-3-yl)amino)-2-fluorophenyl)piperidin-1-yl)cyclohexyl)methyl)-3-((5-fluoro-4-(3-(2-oxopyridin-1(2H)-yl)phenyl)pyrimidin-2-yl)amino)cyclohexane-1-carboxamide (Compound 337)

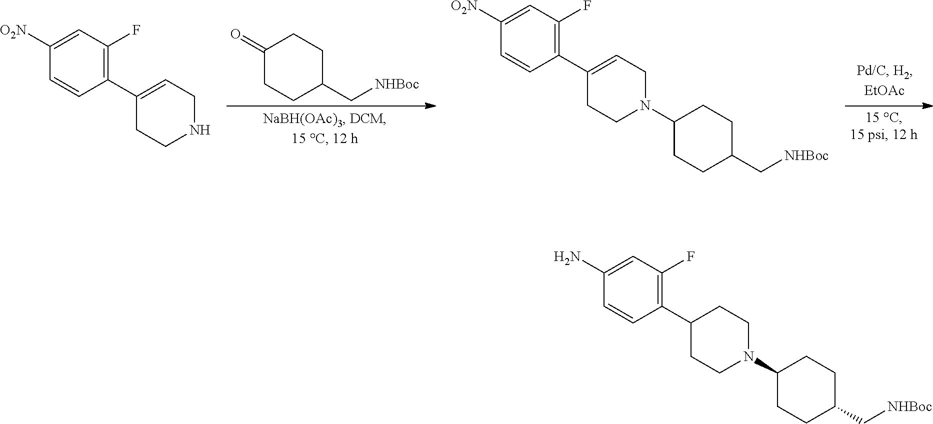 [SMILES: O=[N+]([O-])c1ccc(C2=CCNCC2)c(F)c1 ; CC(C)(C)OC(=O)NCC1CCC(=O)CC1 ; CC(C)(C)OC(=O)NCC1CCC(N2CC=C(c3ccc([N+](=O)[O-])cc3F)CC2)CC1 ; CC(C)(C)OC(=O)NC[C@H]1CC[C@H](N2CCC(c3ccc(N)cc3F)CC2)CC1]

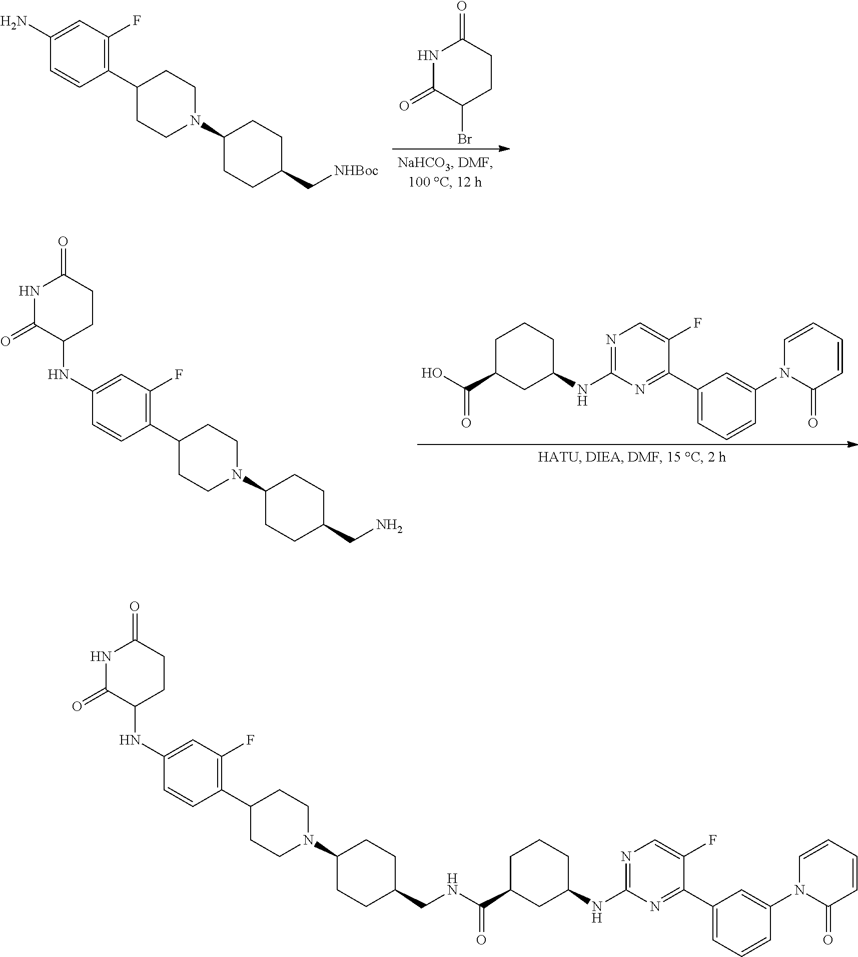 [SMILES: CC(C)(C)OC(=O)NC[C@H]1CC[C@@H](N2CCC(c3ccc(N)cc3F)CC2)CC1 ; O=C1CCC(Br)C(=O)N1 ; NC[C@H]1CC[C@@H](N2CCC(c3ccc(NC4CCC(=O)NC4=O)cc3F)CC2)CC1 ; O=C(O)[C@H]1CCC[C@@H](Nc2ncc(F)c(-c3cccc(-n4ccccc4=O)c3)n2)C1 ; O=C1CCC(Nc2ccc(C3CCN([C@H]4CC[C@@H](CNC(=O)[C@H]5CCC[C@@H](Nc6ncc(F)c(-c7cccc(-n8ccccc8=O)c7)n6)C5)CC4)CC3)c(F)c2)C(=O)N1]

Synthesis of tert-butyl ((4-(4-(2-fluoro-4-nitrophenyl)-3,6-dihydropyridin-1(2H)-yl)cyclohexyl)methyl)carbamate (1290A-2). To a solution of 4-(2-fluoro-4-nitrophenyl)-1,2,3,6-tetrahydropyridine (1290A-1) (1.00 g, 2.97 mmol, 1.00 eq, TFA) and tert-butyl ((4-oxocyclohexyl)methyl)carbamate (676 mg, 2.97 mmol, 1.00 eq) in DCM (20 mL) was added NaBH(OAc)₃ (1.89 g, 8.92 mmol, 3.00 eq) at 15 °C. The mixture was stirred at 15 °C for 12 h. LC-MS showed that 4-(2-fluoro-4-nitrophenyl)-1,2,3,6-tetrahydropyridine (1290A-1) was consumed completely and one main peak with desired mass was detected. The mixture was diluted with H₂O (50 mL) and extracted with DCM (15 mL*3). The combined organic layers were washed with brine (50 mL) dried over Na₂SO₄, filtered and concentrated under reduced pressure. The residue was purified by prep-TLC (SiO₂, EtOAc/MeOH=10/1) to give title compound (1290A-2) (520 mg, 1.20 mmol, 40.3% yield) as a yellow oil. LCMS (ES⁺): m/z 434.2 [M+H]⁺.

Synthesis of trans-tert-butyl (((1r,4r)-4-(4-(4-amino-2-fluorophenyl)piperidin-1-yl)cyclohexyl)methyl)carbamate (1290A-3). To a mixture of 10% Pd/C (200 mg, 50% purity) in EtOAc (5 mL) was added tert-butyl ((4-(4-(2-fluoro-4-nitrophenyl)-3,6-dihydropyridin-1(2H)-yl)cyclohexyl)methyl)carbamate (1290A-2) (520 mg, 1.20 mmol, 1.00 eq) at 15 °C. The mixture was stirred at 15 °C for 12 h under H₂ (15 psi). LC-MS showed that tert-butyl ((4-(4-(2-fluoro-4-nitrophenyl)-3,6-dihydropyridin-1(2H)-yl)cyclohexyl)methyl)carbamate (1290A-2) was consumed completely and one main peak with desired mass was detected. The mixture was filtered and the filtrate was concentrated under reduced pressure to give a residue. The residue was further separated by SFC (column: DAICEL CHIRALCEL OZ 250*25 mm I.D. 10 um; mobile phase: [CO₂—EtOH (0.1% NH₃H₂O)]; B %: 55%, isocratic elution mode) to give trans-tert-butyl (((1r,4r)-4-(4-(4-amino-2-fluorophenyl)piperidin-1-yl)cyclohexyl)methyl)carbamate (1290A-3) (140 mg, 345 μmol, 28.8% yield) and the title compound (1290A-3A) (150 mg, 370 μmol, 30.8% yield) as a yellow oil. LCMS (ES⁺): m/z 406.3 [M+H]⁺.

Synthesis of tert-butyl (((1s,4s)-4-(4-(4-((2,6-dioxopiperidin-3-yl)amino)-2-fluorophenyl)piperidin-1-yl)cyclohexyl)methyl)carbamate (1290A-4) To a solution of tert-butyl (((1s,4s)-4-(4-(4-amino-2-fluorophenyl)piperidin-1-yl)cyclohexyl)methyl)carbamate (1290A-3A) (140 mg, 345 μmol, 1.00 eq) and 3-bromopiperidine-2,6-dione (331 mg, 1.72 mmol, 4.99 eq) in DMF (2 mL) was added NaHCO₃ (58.0 mg, 690 μmol, 26.9 μL, 2.00 eq) at 15 °C. The mixture was stirred at 100 °C for 12 h. LC-MS showed that tert-butyl (((1s,4s)-4-(4-(4-amino-2-fluorophenyl)piperidin-1-yl)cyclohexyl)methyl)carbamate (1290A-3A) was consumed completely and one peak with desired de-Boc mass was detected. The mixture was diluted with H₂O (10 mL) and extracted with EtOAc (5 mL*3). The organic layers were washed with brine (15 mL) dried over Na₂SO₄, filtered and concentrated under reduced pressure. The residue was purified by prep-HPLC (column: Phenomenex luna C18 100*40 mm*5 um; mobile phase: [H₂O (0.2% FA)-ACN]; gradient: 1%-35% B over 8.0 min) to give title compound (1290A-4) (25 mg, 60.0 μmol, 17.4% yield) as a brown solid. LCMS (ES⁺): m/z 417.3 [M+H]⁺.

Synthesis of cis-N-((cis-4-(4-(4-((2,6-dioxopiperidin-3-yl)amino)-2-fluorophenyl)piperidin-1-yl)cyclohexyl)methyl)-3-((5-fluoro-4-(3-(2-oxopyridin-1(2H)-yl)phenyl)pyrimidin-2-yl)amino)cyclohexane-1-carboxamide (Compound 337). To a solution of tert-butyl (((1s,4s)-4-(4-(4-((2,6-dioxopiperidin-3-yl)amino)-2-fluorophenyl)piperidin-1-yl)cyclohexyl)methyl)carbamate (1290A-4) (25 mg, 60.0 μmol, 1.00 eq) and cis-(1R,3S)-3-((5-fluoro-4-(3-(2-oxopyridin-1(2H)-yl)phenyl)pyrimidin-2-yl)amino)cyclohexane-1-carboxylic acid (24.5 mg, 60.0 μmol, 1.00 eq) in DMF (2 mL) was added HATU (45.6 mg, 120 μmol, 2.00 eq) and DIPEA (23.3 mg, 180 μmol, 31.4 μL, 3.00 eq) at 15 °C. The mixture was stirred at 15 °C for 2 h. LC-MS showed that cis-tert-butyl-4-(4-(4-((2,6-dioxopiperidin-3-yl)amino)-2-fluorophenyl)piperidin-1-yl)cyclohexyl)methyl)carbamate (1290A-4) was consumed completely and one peak with desired mass was detected. The mixture was diluted with H₂O (5 mL) and extracted with EtOAc (2 mL*3). The combined organic layers were washed with brine (10 mL) dried over Na₂SO₄, filtered and concentrated under reduced pressure. The residue was purified by prep-HPLC (column: Phenomenex Luna C18 100*30 mm*3 um; mobile phase: [H₂O (0.2% FA)-ACN]; gradient: 15%-55% B over 8.0 min) to give title compound (Compound 337) (17.3 mg, 20.3 μmol, 33.9% yield, 97.0% purity, 0.4 FA) as a white solid. LCMS (ES⁺): m/z 807.3 [M+H]⁺.

### Example 324. Synthesis of cis-N-((trans-4-(4-(4-((2,6-dioxopiperidin-3-yl)amino)-2-fluorophenyl)piperazin-1-yl)cyclohexyl)methyl)-3-((5-fluoro-4-(3-(2-oxopyridin-1(2H)-yl)phenyl)pyrimidin-2-yl)amino)cyclohexane-1-carboxamide (Compound 352)

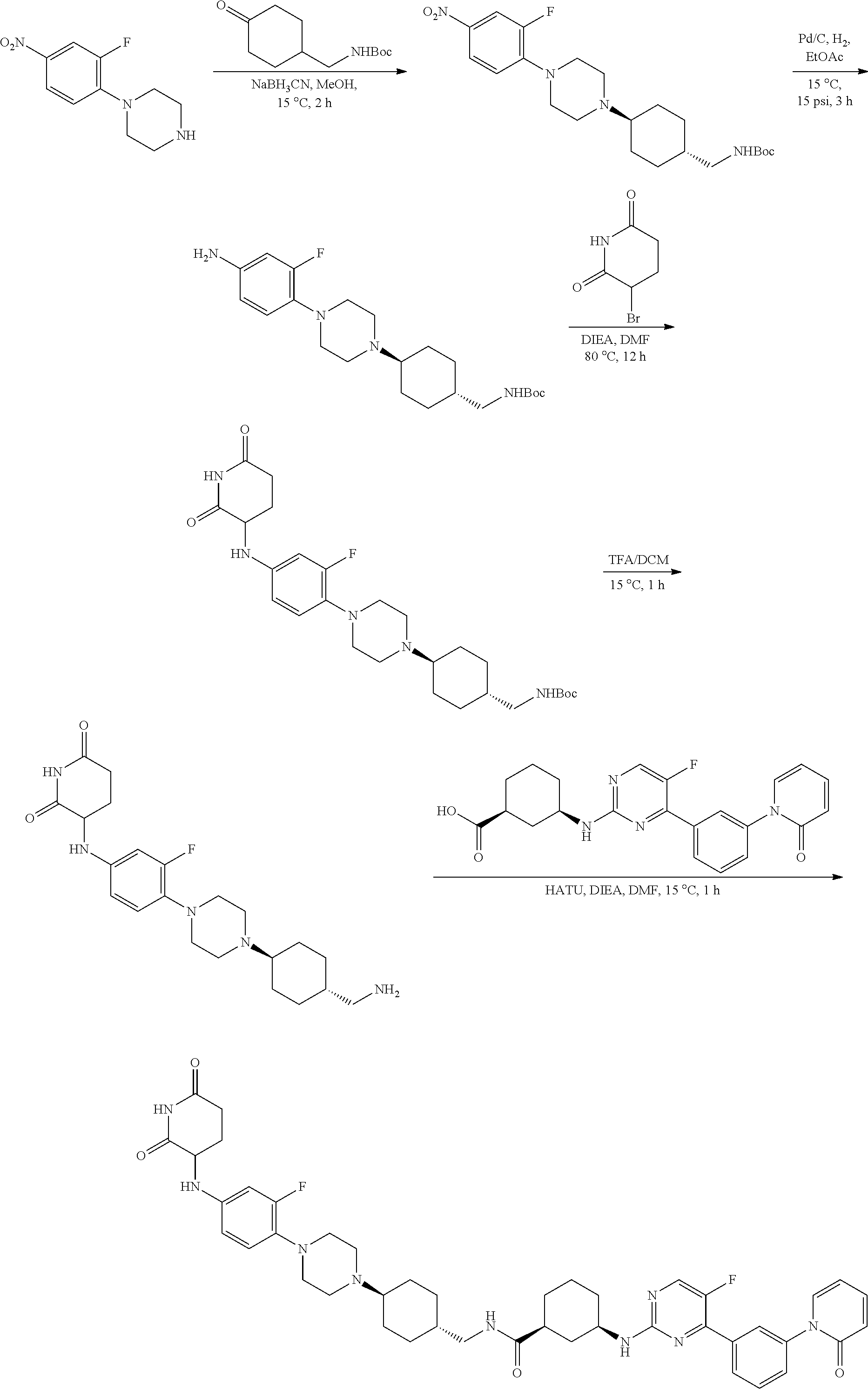

Synthesis of trans-tert-butyl (((1r,4r)-4-(4-(2-fluoro-4-nitrophenyl)piperazin-1-yl)cyclohexyl)methyl)carbamate. To a solution of 1-(2-fluoro-4-nitrophenyl)piperazine (5.00 g, 22.2 mmol, 1.00 eq) in MeOH (50.0 mL) were added tert-butyl ((4-oxocyclohexyl)methyl)carbamate (10.0 g, 44.4 mmol, 2.00 eq), AcOH (2.67 g, 44.4 mmol, 2.54 mL, 2.00 eq) and NaBH₃CN (2.79 g, 44.4 mmol, 2.00 eq) at 15 °C. The mixture was stirred at 15 °C for 2 h. LC-MS showed that 1-(2-fluoro-4-nitrophenyl)piperazine was consumed completely and the desired mass was detected. The mixture was concentrated under reduced pressure to give a residue. The residue was further separated by SFC (column: DAICEL CHIRALCEL OZ 250*25 mm I.D. 10 um; mobile phase: [CO₂-MeOH (0.1% NH₃H₂O)]; B %: 50%, isocratic elution mode). Compound 2 (400 mg, 886 μmol, 3.99% yield, 96.7% purity) was obtained as a yellow solid. LCMS (ES⁺): m/z 437.2 [M+H]⁺.

Synthesis of trans-tert-butyl (((1r,4r)-4-(4-(4-amino-2-fluorophenyl)piperazin-1-yl)cyclohexyl)methyl)carbamate. To a solution of trans-tert-butyl (((1r,4r)-4-(4-(2-fluoro-4-nitrophenyl)piperazin-1-yl)cyclohexyl)methyl)carbamate (400 mg, 916 μmol, 1.00 eq) in EtOAc (2.00 mL) was added 10% Pd/C (480 mg, 50.0% purity) at 15 °C. The mixture was stirred at 15 °C for 3 h under H₂ (15 Psi). LC-MS showed that trans-tert-butyl (((1r,4r)-4-(4-(2-fluoro-4-nitrophenyl)piperazin-1-yl)cyclohexyl)methyl)carbamate was consumed completely, and the desired mass was detected. The mixture was filtered and the filtrate was concentrated under reduced pressure to give title Compound (400 mg, crude) was obtained as a yellow oil. LCMS (ES⁺): m/z 407.2[M+H]⁺.

Synthesis of trans-tert-butyl (((1r,4r)-4-(4-(4-((2,6-dioxopiperidin-3-yl)amino)-2-fluorophenyl)piperazin-1-yl)cyclohexyl)methyl)carbamate. To a solution of trans-tert-butyl (((1r,4r)-4-(4-(4-amino-2-fluorophenyl)piperazin-1-yl)cyclohexyl)methyl)carbamate (390 mg, 959 μmol, 1.00 eq) in DMF (5.00 mL) were added DIPEA (371 mg, 2.88 mmol, 501 μL, 3.00 eq) and 3-bromopiperidine-2,6-dione (736 mg, 3.84 mmol, 4.00 eq) at 15 °C. The mixture was stirred at 80 °C for 12 h. LC-MS showed that trans-tert-butyl (((1r,4r)-4-(4-(4-amino-2-fluorophenyl)piperazin-1-yl)cyclohexyl)methyl)carbamate was consumed completely, and the desired mass was detected. The mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 15%-50% B over 8.0 min) to give title Compound (200 mg, 386 μmol, 40.2% yield) as a yellow solid. LCMS (ES⁺): m/z 518.3[M+H]⁺.

Synthesis of trans-3-((4-(4-((1r,4r)-4-(aminomethyl)cyclohexyl)piperazin-1-yl)-3-fluorophenyl)amino)piperidine-2,6-dione. To a solution of trans-tert-butyl (((1r,4r)-4-(4-(4-((2,6-dioxopiperidin-3-yl)amino)-2-fluorophenyl)piperazin-1-yl)cyclohexyl)methyl)carbamate (190 mg, 367 μmol, 1.00 eq) in DCM (3 mL) was added TFA (4.61 g, 40.4 mmol, 3 mL, 110 eq) at 0 °C. The mixture was stirred at 15 °C for 1 h. LC-MS showed that trans-tert-butyl (((1r,4r)-4-(4-(4-((2,6-dioxopiperidin-3-yl)amino)-2-fluorophenyl)piperazin-1-yl)cyclohexyl)methyl)carbamate was consumed completely, and the desired mass was detected. The mixture was concentrated under reduced pressure to give title Compound (190 mg, crude, TFA) as a yellow oil. LCMS (ES⁺): m/z 418.2[M+H]⁺.

Synthesis of cis-N-((trans-4-(4-(4-((2,6-dioxopiperidin-3-yl)amino)-2-fluorophenyl)piperazin-1-yl)cyclohexyl)methyl)-3-((5-fluoro-4-(3-(2-oxopyridin-1(2H)-yl)phenyl)pyrimidin-2-yl)amino)cyclohexane-1-carboxamide (Compound 352). To a solution of trans-3-((4-(4-((1r,4r)-4-(aminomethyl)cyclohexyl)piperazin-1-yl)-3-fluorophenyl)amino)piperidine-2,6-dione (180 mg, 338 μmol, 1.00 eq, TFA) in DMF (2.00 mL) were added DIPEA (131 mg, 1.02 mmol, 176 μL, 3.00 eq), cis-(1R,3S)-3-((5-fluoro-4-(3-(2-oxopyridin-1(2H)-yl)phenyl)pyrimidin-2-yl)amino)cyclohexane-1-carboxylic acid (138 mg, 338 μmol, 1.00 eq) and HATU (193 mg, 507 μmol, 1.50 eq) at 15 °C. The mixture was stirred at 15 °C for 1 h. LC-MS showed that trans-3-((4-(4-((1r,4r)-4-(aminomethyl)cyclohexyl)piperazin-1-yl)-3-fluorophenyl)amino)piperidine-2,6-dione was consumed completely, and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to give title Compound (Compound 352) (7.30 mg, 9.04 μmol, 2.67% yield, 100.0% purity) as a gray solid. LCMS (ES⁺): m/z 808.4 [M+H]⁺.

### Example 325. Synthesis of cis-3-(6-(1-(2-(1-(3-((5-fluoro-4-(3-(2-oxopyridin-1(2H)-yl)phenyl)pyrimidin-2-yl)amino)cyclohexane-1-carbonyl)-4-hydroxypiperidin-4-yl)ethyl)piperidin-4-yl)-2-oxobenzo[d]oxazol-3 (2H)-yl)piperidine-2,6-dione (Compound 347)

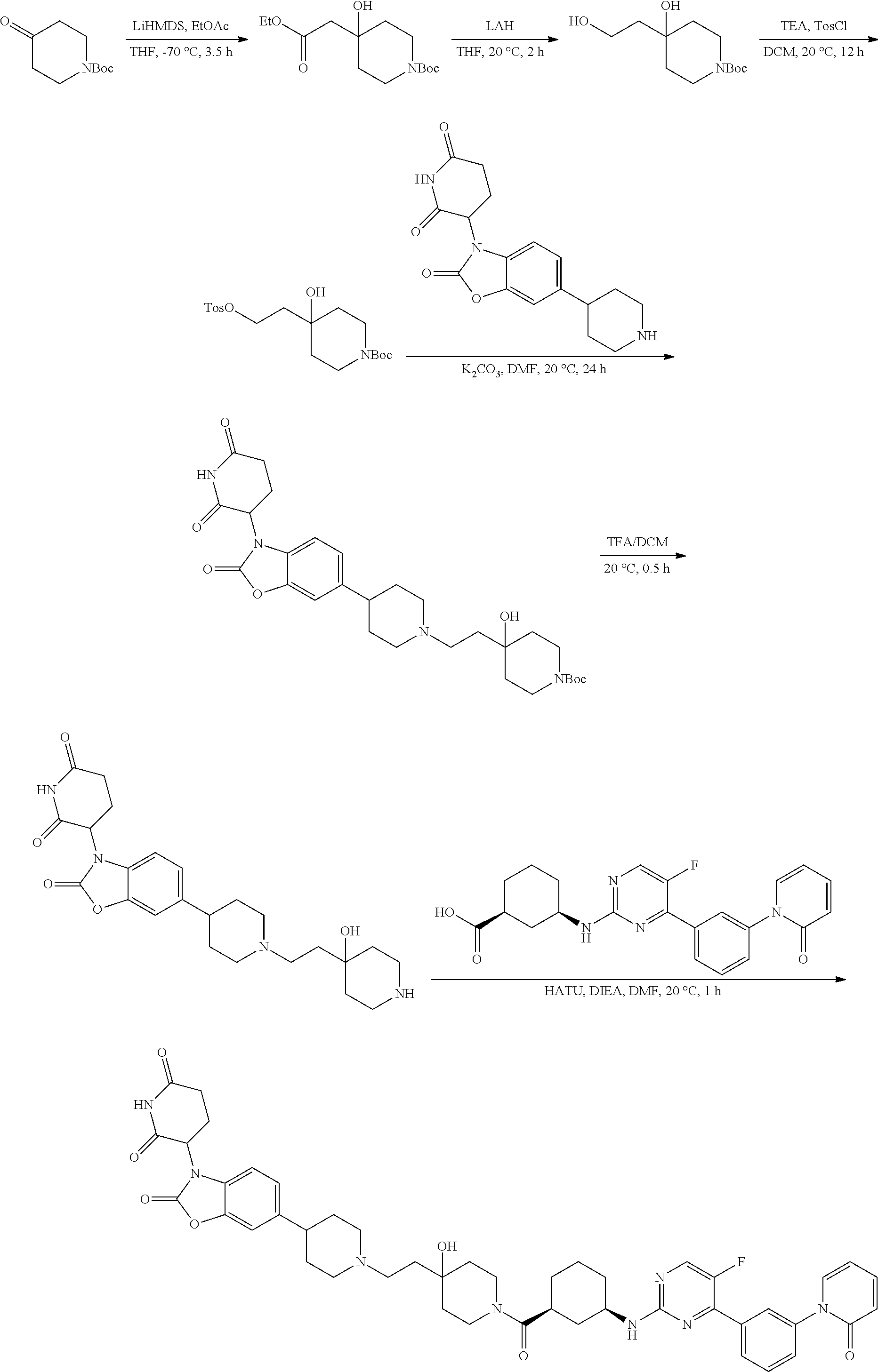

Synthesis of tert-butyl 4-(2-ethoxy-2-oxoethyl)-4-hydroxypiperidine-1-carboxylate. A solution of LiHMDS (1 M, 27.4 mL, 1.09 eq) in THF (50 mL) was cooled to −70 °C under N₂ atmosphere. Then EtOAc (2.40 g, 27.3 mmol, 2.67 mL, 1.09 eq) was added to the mixture at −70 °C. The mixture was stirred at −70 °C for 30 min. Then tert-butyl 4-oxopiperidine-1-carboxylate (5.00 g, 25.1 mmol, 1.00 eq) in THF (25 mL) was slowly added dropwise to the mixture at −70 °C. The mixture was stirred at −70 °C for 3 h. The product couldn't detected by LCMS. TLC (SiO₂, Petroleum ether:Ethyl acetate=1:1) indicated tert-butyl 4-oxopiperidine-1-carboxylate was consumed completely and one new spot formed. The mixture was warmed to 0 °C. Water (45 mL) was added to the mixture dropwise to quench the reaction at 0 °C. Then the reaction mixture was extracted with EtOAc (40 mL*3). The organic layers were dried over anhydrous sodium sulfate, filtered and the filtrate was concentrated under reduced pressure to give the title compound (3.50 g, crude) as a white solid. The product couldn't be detected by LCMS. It was confirmed by ¹H NMR (400 MHz, CHLOROFORM-d) δ=4.13 (q, J=7.1 Hz, 2H), 3.84-3.68 (m, 2H), 3.56 (s, 1H), 3.15 (br s, 2H), 2.41 (s, 2H), 1.61 (br d, J=12.6 Hz, 2H), 1.48-1.42 (m, 2H), 1.40 (s, 9H), 1.23 (t, J=7.2 Hz, 3H).

Synthesis of tert-butyl 4-hydroxy-4-(2-hydroxyethyl)piperidine-1-carboxylate. To a solution of tert-butyl 4-(2-ethoxy-2-oxoethyl)-4-hydroxypiperidine-1-carboxylate (3.50 g, 12.2 mmol, 1.00 eq) in THF (70 mL) was added LAH (2 M, 6.70 mL, 1.10 eq) at 0 °C under N₂ atmosphere. The mixture was stirred at 20 °C for 2 h. LCMS showed the desired mass was detected. TLC (SiO₂, Petroleum ether:Ethyl acetate=1:1) indicated tert-butyl 4-(2-ethoxy-2-oxoethyl)-4-hydroxypiperidine-1-carboxylate was consumed completely and two new spots formed. The mixture was quenched with H₂O (0.73 mL), NaOH (15%, 0.73 mL) and H₂O (2.19 mL) at 0 °C. Then the suspension was filtered, the filter cake was washed with EtOAc (50 mL). Then H₂O (50 mL) was added to the filtrate, the resulting mixture was extracted with ethyl acetate (50 mL*3). The combined organic layers were dried over Na₂SO₄, filtered and the filtrate was concentrated under reduced pressure to give the title compound (2.50 g, crude) as a yellow oil. LCMS (ES⁺): m/z 146.2 [M+H−100]⁺.

Synthesis of tert-butyl 4-hydroxy-4-(2-(tosyloxy)ethyl)piperidine-1-carboxylate. To a solution of tert-butyl 4-hydroxy-4-(2-hydroxyethyl)piperidine-1-carboxylate (2.50 g, 10.2 mmol, 1.00 eq) in DCM (50 mL) were added TosCl (1.94 g, 10.2 mmol, 1.00 eq) and TEA (3.09 g, 30.6 mmol, 4.26 mL, 3.00 eq) at 0 °C under N₂ atmosphere. The mixture was stirred at 20 °C for 12 h under N₂. LC-MS showed tert-butyl 4-hydroxy-4-(2-hydroxyethyl)piperidine-1-carboxylate was consumed completely and the desired mass was detected. Water (50 mL) was added to the mixture at 20 °C. Then the reaction mixture was extracted with DCM (30 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=3:1 to 1:1) to give the title compound (2.60 g, 6.51 mmol, 63.9% yield) as a yellow oil. LCMS (ES⁺): m/z 300.1 [M+H−100]⁺.

Synthesis of tert-butyl 4-(2-(4-(3-(2,6-dioxopiperidin-3-yl)-2-oxo-2,3-dihydrobenzo[d]oxazol-6-yl)piperidin-1-yl)ethyl)-4-hydroxypiperidine-1-carboxylate. To a solution of tert-butyl 4-hydroxy-4-(2-(tosyloxy)ethyl)piperidine-1-carboxylate (1.60 g, 4.01 mmol, 1.00 eq) in DMF (50 mL) were added 3-[2-oxo-6-(4-piperidyl)-1,3-benzoxazol-3-yl]piperidine-2,6-dione (4.44 g, 10.0 mmol, 2.50 eq, TFA) and K₂CO₃ (2.77 g, 20.0 mmol, 5.00 eq) at 20 °C. The mixture was stirred at 20 °C for 24 h. LC-MS showed ~8% of tert-butyl 4-hydroxy-4-(2-(tosyloxy)ethyl)piperidine-1-carboxylate was consumed completely and the desired mass was detected. Water (50 mL) was added to the mixture at 20 °C. Then the reaction mixture was extracted with EtOAc (30 mL*3). The combined organic layers were washed with brine (100 mL), then the organic layer was dried over anhydrous sodium sulfate, filtered and the filtrate was concentrated under reduced pressure to give a residue. The residue was purified by column chromatography (SiO₂, Petroleum ether:Ethyl acetate=1:1 to 1:2) to give the title compound (200 mg, 359 μmol, 8.97% yield) as a white solid. LCMS (ES⁺): m/z 557.4[M+H]⁺.

Synthesis of 3-(6-(1-(2-(4-hydroxypiperidin-4-yl)ethyl)piperidin-4-yl)-2-oxobenzo[d]oxazol-3 (2H)-yl)piperidine-2,6-dione. To a solution of tert-butyl 4-(2-(4-(3-(2,6-dioxopiperidin-3-yl)-2-oxo-2,3-dihydrobenzo[d]oxazol-6-yl)piperidin-1-yl)ethyl)-4-hydroxypiperidine-1-carboxylate (200 mg, 359 μmol, 1.00 eq) in DCM (3 mL) was added TFA (9.21 g, 80.8 mmol, 1.00 mL, 225 eq) at 20 °C. The mixture was stirred at 20 °C for 0.5 h. LC-MS showed tert-butyl 4-(2-(4-(3-(2,6-dioxopiperidin-3-yl)-2-oxo-2,3-dihydrobenzo[d]oxazol-6-yl)piperidin-1-yl)ethyl)-4-hydroxypiperidine-1-carboxylate was consumed completely and the desired mass was detected. The reaction mixture was concentrated under reduced pressure to give the title compound (200 mg, crude, TFA) as a yellow oil. LCMS (ES⁺): m/z 457.2 [M+H]⁺.

Synthesis of cis-3-(6-(1-(2-(1-(3-((5-fluoro-4-(3-(2-oxopyridin-1(2H)-yl)phenyl)pyrimidin-2-yl)amino)cyclohexane-1-carbonyl)-4-hydroxypiperidin-4-yl)ethyl)piperidin-4-yl)-2-oxobenzo[d]oxazol-3 (2H)-yl)piperidine-2,6-dione (Compound 347). To a solution of cis-3-((5-fluoro-4-(3-(2-oxopyridin-1(2H)-yl)phenyl)pyrimidin-2-yl)amino)cyclohexane-1-carboxylic acid (12.5 mg, 30.7 μmol, 0.100 eq) in DMF (3 mL) were added HATU (175 mg, 460 μmol, 1.50 eq), 3-(6-(1-(2-(4-hydroxypiperidin-4-yl)ethyl)piperidin-4-yl)-2-oxobenzo[d]oxazol-3 (2H)-yl)piperidine-2,6-dione (175 mg, 307 μmol, 1.00 eq, TFA) and DIEA (198 mg, 1.53 mmol, 267 μL, 5.00 eq) at 20 °C. The mixture was stirred at 20 °C for 1 h. LC-MS showed 3-(6-(1-(2-(4-hydroxypiperidin-4-yl)ethyl)piperidin-4-yl)-2-oxobenzo[d]oxazol-3 (2H)-yl)piperidine-2,6-dione was consumed completely and the desired mass was detected. The reaction mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 35%-65% B over 8.0 min) to give the title compound (Compound 347) (5.60 mg, 6.19 μmol, 2.02% yield, 93.7% purity) as a white solid. LCMS (ES⁺): m/z 847.3 [M+H]⁺

### Example 326. Synthesis of cis-3-((3-fluoro-4-(1-(2-(1-(3-((5-fluoro-4-(3-(2-oxopyridin-1(2H)-yl)phenyl)pyrimidin-2-yl)amino)cyclohexane-1-carbonyl)-4-hydroxypiperidin-4-yl)ethyl)piperidin-4-yl)phenyl)amino)piperidine-2,6-dione (Compound 348)

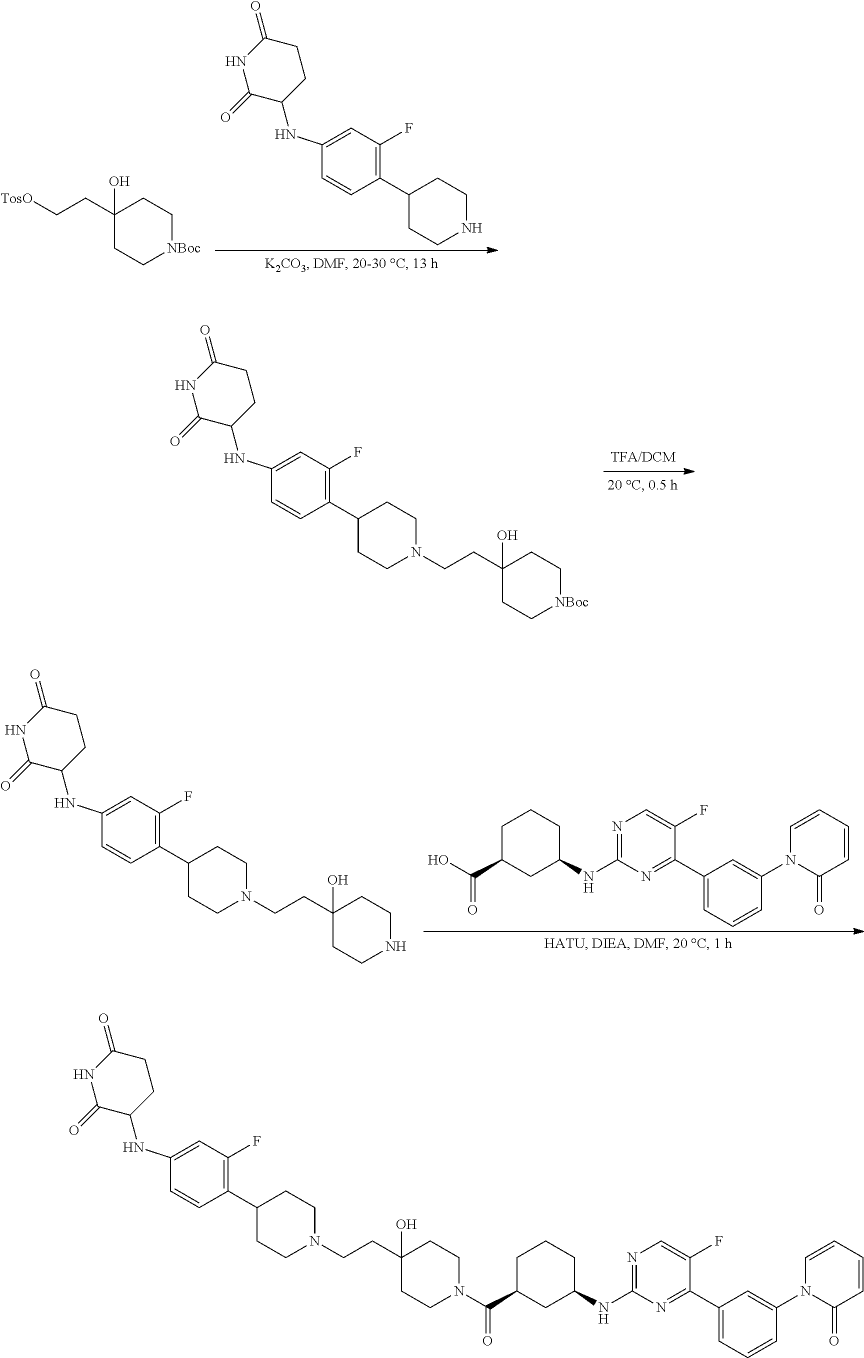

Synthesis of tert-butyl 4-(2-(4-(4-((2,6-dioxopiperidin-3-yl)amino)-2-fluorophenyl)piperidin-1-yl)ethyl)-4-hydroxypiperidine-1-carboxylate. To a solution of tert-butyl 4-hydroxy-4-(2-(tosyloxy)ethyl)piperidine-1-carboxylate (1.00 g, 2.50 mmol, 1.00 eq) in DMF (30 mL) were added 3-[3-fluoro-4-(4-piperidyl)anilino]piperidine-2,6-dione (2.62 g, 6.26 mmol, 2.50 eq, TFA) and K₂CO₃ (1.73 g, 12.5 mmol, 5.00 eq) at 20 °C. The mixture was stirred at 30 °C for 13 h. LC-MS showed tert-butyl 4-hydroxy-4-(2-(tosyloxy)ethyl)piperidine-1-carboxylate was consumed completely and the desired mass was detected. Water (20 mL) was added to the mixture at 20 °C. Then the reaction mixture was extracted with EtOAc (10 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=1:1 to 1:2) to give the title compound (200 mg, 375 μmol, 15.0% yield) as a white solid. LCMS (ES⁺): m/z 533.4 [M+H]⁺.

Synthesis of 3-((3-fluoro-4-(1-(2-(4-hydroxypiperidin-4-yl)ethyl)piperidin-4-yl)phenyl)amino)piperidine-2,6-dione. To a solution of tert-butyl 4-(2-(4-(4-((2,6-dioxopiperidin-3-yl)amino)-2-fluorophenyl)piperidin-1-yl)ethyl)-4-hydroxypiperidine-1-carboxylate (200 mg, 375 μmol, 1.00 eq) in DCM (3 mL) was added TFA (1.54 g, 13.5 mmol, 1 mL, 35.9 eq) at 20 °C. The mixture was stirred at 20 °C for 0.5 h. LC-MS showed tert-butyl 4-(2-(4-(4-((2,6-dioxopiperidin-3-yl)amino)-2-fluorophenyl)piperidin-1-yl)ethyl)-4-hydroxypiperidine-1-carboxylate was consumed completely and the desired mass was detected. The reaction mixture was concentrated under reduced pressure to give the title compound (200 mg, crude, TFA) as a brown oil. LCMS (ES⁺): m/z 433.3 [M+H]⁺.

Synthesis of cis-3-((3-fluoro-4-(1-(2-(1-(3-((5-fluoro-4-(3-(2-oxopyridin-1(2H)-yl)phenyl)pyrimidin-2-yl)amino)cyclohexane-1-carbonyl)-4-hydroxypiperidin-4-yl)ethyl)piperidin-4-yl)phenyl)amino)piperidine-2,6-dione (Compound 348). To a solution of cis-3-((5-fluoro-4-(3-(2-oxopyridin-1(2H)-yl)phenyl)pyrimidin-2-yl)amino)cyclohexane-1-carboxylic acid (44.8 mg, 110 μmol, 0.300 eq) in DMF (3 mL) were added HATU (209 mg, 549 μmol, 1.50 eq), 3-((3-fluoro-4-(1-(2-(4-hydroxypiperidin-4-yl)ethyl)piperidin-4-yl)phenyl)amino)piperidine-2,6-dione (200 mg, 366 μmol, 1.00 eq, TFA) and DIEA (237 mg, 1.83 mmol, 319 μL, 5.00 eq) at 20 °C. The mixture was stirred at 20 °C for 1 h. LC-MS showed 3-((3-fluoro-4-(1-(2-(4-hydroxypiperidin-4-yl)ethyl)piperidin-4-yl)phenyl)amino)piperidine-2,6-dione was consumed completely and the desired mass was detected. The reaction mixture was concentrated under reduced pressure give a residue. The residue was purified by prep-HPLC (column: Waters Xbridge Prep OBD C18 150*40 mm*10 um; mobile phase: [H₂O (10 mM NH₄HCO₃)-ACN]; gradient: 35%-75% B over 8.0 min) to give the title compound (Compound 348) (5.60 mg, 6.80 μmol, 1.86% yield, 100% purity) as a yellow solid. LCMS (ES⁺): m/z 823.5 [M+H]⁺

### Example 327. Synthesis of cis-N-(1-((1-(3-(2,6-dioxopiperidin-3-yl)-2-oxo-2,3-dihydrobenzo[d]oxazol-6-yl)piperidin-4-yl)methyl)piperidin-4-yl)-3-((5-fluoro-4-(3-(2-oxopyridin-1(2H)-yl)phenyl)pyrimidin-2-yl)amino)cyclohexane-1-carboxamide (Compound 320)

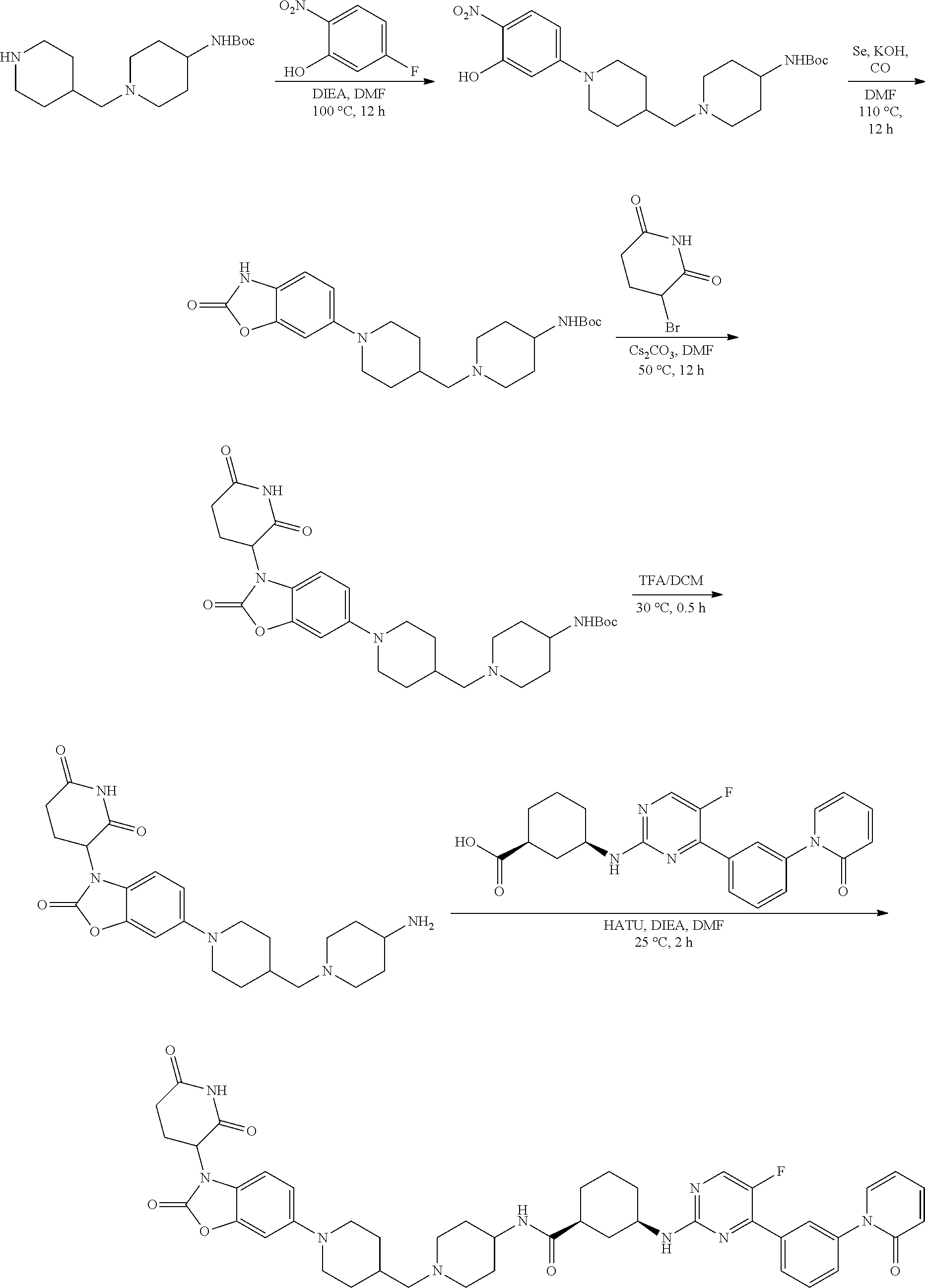

Synthesis of tert-butyl (1-((1-(3-hydroxy-4-nitrophenyl)piperidin-4-yl)methyl)piperidin-4-yl)carbamate. To a solution of tert-butyl (1-(piperidin-4-ylmethyl)piperidin-4-yl)carbamate (9.00 g, 30.3 mmol, 1.00 eq) and 5-fluoro-2-nitrophenol (4.75 g, 30.3 mmol, 1.00 eq) in DMF (120 mL) was added DIEA (11.7 g, 90.8 mmol, 15.8 mL, 3.00 eq) at 20 °C. The mixture was stirred at 100 °C for 12 h. LCMS showed tert-butyl (1-(piperidin-4-ylmethyl)piperidin-4-yl)carbamate was consumed completely and the desired mass was detected, H₂O (80 mL) was added to the solution, then the mixture was extracted with EtOAc (80 mL*3), the combined organic layers were washed with brine (100 mL), dried over Na₂SO₄, filtered and the filtered was concentrated under reduced pressure to give a residue. The residue was purified by MPLC (SiO₂, Dichloromethane/Methanol=1/0 to 1/1) to give the title compound (5.00 g, 11.5 mmol, 38.0% yield) as a yellow solid. LCMS (ES⁺): m/z 435.2 [M+H]⁺.

Synthesis of tert-butyl (1-((1-(2-oxo-2,3-dihydrobenzo[d]oxazol-6-yl)piperidin-4-yl)methyl)piperidin-4-yl)carbamate. To a solution of tert-butyl (1-((1-(3-hydroxy-4-nitrophenyl)piperidin-4-yl)methyl)piperidin-4-yl)carbamate (5.00 g, 11.2 mmol, 1.00 eq) in DMF (60 mL) were added KOH (1.29 g, 23.0 mmol, 2.00 eq) and Se (909 mg, 11.2 mmol, 891 μL, 1.00 eq) at 25 °C under N₂. The mixture was degassed and purged with CO for three times and the resulting mixture was stirred under CO (15 psi) at 110 °C for 12 h. LCMS showed tert-butyl (1-((1-(3-hydroxy-4-nitrophenyl)piperidin-4-yl)methyl)piperidin-4-yl)carbamate was consumed completely the desired mass was detected, H₂O (60 mL) was added to the solation, the mixture was extracted with EtOAc (60 mL*3), then the combined organic layers were washed with brine (800 mL), dried over Na₂SO₄, filtered and the filtrate was concentrated under reduced pressure to give a residue. The residue was purified by MPLC (SiO₂, Dichloromethane/Methanol=1/0 to 5/1) to give the title compound (3.00 g, 6.97 mmol, 60.6% yield) as a yellow solid. LCMS (ES⁺): m/z 431.2 [M+H]⁺.

Synthesis of tert-butyl (1-((1-(3-(2,6-dioxopiperidin-3-yl)-2-oxo-2,3-dihydrobenzo[d]oxazol-6-yl)piperidin-4-yl)methyl)piperidin-4-yl)carbamate. To a stirred solution of tert-butyl (1-((1-(2-oxo-2,3-dihydrobenzo[d]oxazol-6-yl)piperidin-4-yl)methyl)piperidin-4-yl)carbamate (1.80 g, 4.18 mmol, 1.00 eq) in DMF (20 mL) were added Cs₂CO₃ (2.72 g, 8.36 mmol, 2.00 eq) and 3-bromopiperidine-2,6-dione (2.41 g, 12.5 mmol, 3.00 eq) at 25 °C. The mixture was stirred at 50 °C for 12 h. LCMS showed tert-butyl (1-((1-(2-oxo-2,3-dihydrobenzo[d]oxazol-6-yl)piperidin-4-yl)methyl)piperidin-4-yl)carbamate was consumed completely the desired mass was detected. H₂O (20 mL) was added to the solution, then the mixture was extracted with EtOAc (20 mL*3), the combined organic layers were washed with brine (15 mL*3), dried over Na₂SO₄, filtered and the filtered was concentrated under reduced pressure to give a residue. The residue was purified by MPLC (SiO₂. Dichloromethane:Methanol=1/0 to 1/1) to give the title compound (200 mg. 369 μmol, 8.83% yield) as a white solid, LCMS (ES⁺): m/z 542.2 [M+H]⁺.

Synthesis of 3-(6-(4-((4-aminopiperidin-1-yl)methyl)piperidin-1-yl)-2-oxobenzo[d]oxazol-3 (2H)-yl)piperidine-2,6-dione. To a solution of tert-butyl (1-((1-(3-(2,6-dioxopiperidin-3-yl)-2-oxo-2,3-dihydrobenzo[d]oxazol-6-yl)piperidin-4-yl)methyl)piperidin-4-yl)carbamate (130 mg, 240 μmol, 1.00 eq) in DCM (1.5 mL) was added dropwise TFA (0.5 mL) at 25 °C. The resulting mixture was stirred at 30 °C for 0.5 h. LCMS showed tert-butyl (1-((1-(3-(2,6-dioxopiperidin-3-yl)-2-oxo-2,3-dihydrobenzo[d]oxazol-6-yl)piperidin-4-yl)methyl)piperidin-4-yl)carbamate was consumed completely and the desired mass was detected, The mixture was concentrated under reduced pressure to give the title compound (130 mg, crude, TFA) as a yellow oil. LCMS (ES⁺): m/z 442.2 [M+H]⁺.

Synthesis of cis-N-(1-((1-(3-(2,6-dioxopiperidin-3-yl)-2-oxo-2,3-dihydrobenzo[d]oxazol-6-yl)piperidin-4-yl)methyl)piperidin-4-yl)-3-((5-fluoro-4-(3-(2-oxopyridin-1(2H)-yl)phenyl)pyrimidin-2-yl)amino)cyclohexane-1-carboxamide (Compound 320). To a solution of 3-(6-(4-((4-aminopiperidin-1-yl)methyl)piperidin-1-yl)-2-oxobenzo[d]oxazol-3 (2H)-yl)piperidine-2,6-dione (130 mg, 234 μmol, 1.00 eq, TFA) in DMF (3 mL) were added HATU (133 mg, 351 μmol, 1.50 eq) and DIEA (90.7 mg, 702 μmol, 122 μL, 3.00 eq) at 25 °C, and then cis-3-((5-fluoro-4-(3-(2-oxopyridin-1(2H)-yl)phenyl)pyrimidin-2-yl)amino)cyclohexane-1-carboxylic acid (95.6 mg, 234 μmol, 1.00 eq) was added at 25 °C. The resulting mixture was stirred at 25 °C for 2 hr. LCM S showed that 3-(6-(4-((4-aminopiperidin-1-yl)methyl)piperidin-1-yl)-2-oxobenzo[d]oxazol-3 (2H)-yl)piperidine-2,6-dione was consumed completely the desired mass was detected. The mixture was filtered, and the filtrate was concentrated under reduced pressure to give a residue. The residue was purified by prep-HPLC (column: Waters Xbridge BEH C18 100*30 mm 5 um; mobile phase: [H₂O (10 mM NH₄HCO₃)-ACN]; gradient: 21%-51% B over 8.0 min) to give the title compound (Compound 320) (6.80 mg, 7.60 μmol, 3.25% yield, 93.0% purity) as a yellow solid. LCMS (ES⁺): m/s 832.4 [M+H]⁺

### Example 328. Synthesis of cis-N-(1-((1-(3-(2,6-dioxopiperidin-3-yl)-1-methyl-1H-indazol-6-yl)piperidin-4-yl)methyl)piperidin-4-yl)-3-((5-fluoro-4-(3-(2-oxopyridin-1(2H)-yl)phenyl)pyrimidin-2-yl)amino)cyclohexane-1-carboxamide (Compound 332)

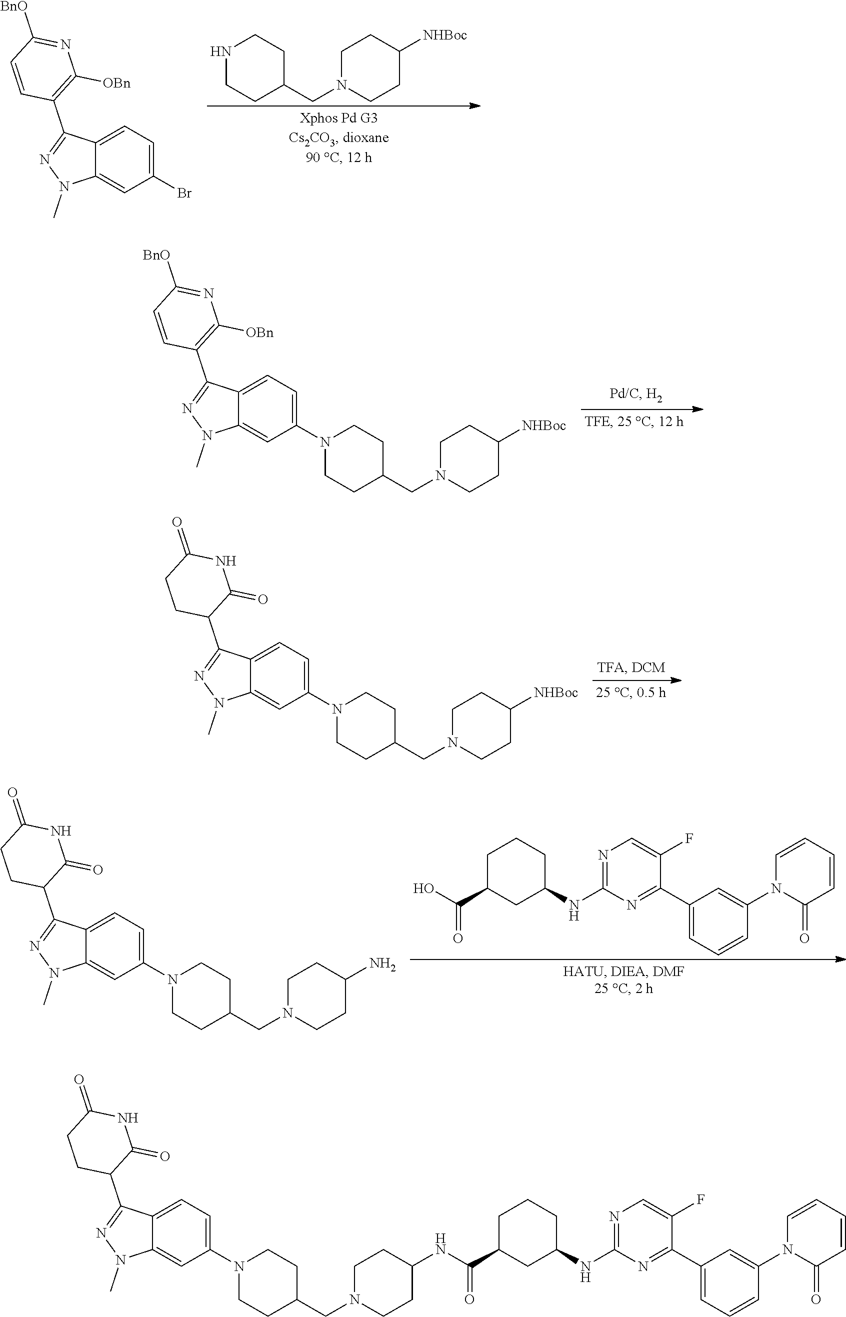

Synthesis of tert-butyl (1-((1-(3-(2,6-bis(benzyloxy)pyridin-3-yl)-1-methyl-1H-indazol-6-yl)piperidin-4-yl)methyl)piperidin-4-yl)carbamate. To a solution of 3-(2,6-bis(benzyloxy)pyridin-3-yl)-6-bromo-1-methyl-1H-indazole (1.87 g, 3.74 mmol, 1.00 eq) and tert-butyl (1-(piperidin-4-ylmethyl)piperidin-4-yl)carbamate (1.11 g, 3.74 mmol, 1.00 eq) in dioxane (25 mL) were added Xphos Pd G3 (316 mg, 374 μmol, 0.100 eq) and Cs₂CO₃ (3.65 g, 11.2 mmol, 3.00 eq) at 20 °C under N₂. The mixture was stirred at 90 °C for 12 h under N₂ atmosphere. LCMS showed 3-(2,6-bis(benzyloxy)pyridin-3-yl)-6-bromo-1-methyl-1H-indazole was consumed completely the desired mass was detected. H₂O (20 mL) was added to the solution, then the mixture was extracted with EtOAc (20 mL*3), the combined organic layers were dried over Na₂SO₄, filtered and the filtrate was concentrated under reduced pressure to give a residue. The residue was purified by MPLC (SiO₂, Petroleum ether/Ethyl acetate=1/0 to 1/1) to give the title compound (300 mg, 418 μmol, 11.2% yield) as a yellow solid. LCMS (ES⁺): m/z 717.5 [M+H]⁺.

Synthesis of tert-butyl (1-((1-(3-(2,6-dioxopiperidin-3-yl)-1-methyl-1H-indazol-6-yl)piperidin-4-yl)methyl)piperidin-4-yl)carbamate. To a solution of tert-butyl (1-((1-(3-(2,6-bis(benzyloxy)pyridin-3-yl)-1-methyl-1H-indazol-6-yl)piperidin-4-yl)methyl)piperidin-4-yl)carbamate (300 mg, 418 μmol, 1.00 eq) in TFE (4 mL) was added 10% Pd/C (300 mg, 50.0% purity) at 25 °C under N₂, the mixture was degassed and purged with H₂ for three times and the resulting mixture was stirred at 25 °C for 12 h under H₂ (15 psi). LCMS showed that tert-butyl (1-((1-(3-(2,6-bis(benzyloxy)pyridin-3-yl)-1-methyl-1H-indazol-6-yl)piperidin-4-yl)methyl)piperidin-4-yl)carbamate was consumed completely the desired mass was detected. The mixture was filtered and the filtrate was concentrated under reduced pressure to give the title compound (300 mg, crude) as a yellow solid. LCMS (ES⁺): m/z 539.4 [M+H]⁺.

Synthesis of 3-(6-(4-((4-(l2-azaneyl)piperidin-1-yl)methyl)piperidin-1-yl)-1-methyl-1H-indazol-3-yl)piperidine-2,6-dione. To a solution of tert-butyl (1-((1-(3-(2,6-dioxopiperidin-3-yl)-1-methyl-1H-indazol-6-yl)piperidin-4-yl)methyl)piperidin-4-yl)carbamate (330 mg, 613 μmol, 1.00 eq) in DCM (3 mL) was added dropwise TFA (1 mL) at 25 °C. The resulting mixture was stirred at 25 °C for 0.5 h. LCMS showed that tert-butyl (1-((1-(3-(2,6-dioxopiperidin-3-yl)-1-methyl-1H-indazol-6-yl)piperidin-4-yl)methyl)piperidin-4-yl)carbamate was consumed completely the desired mass was detected. The mixture was concentrated under reduced pressure to give the title compound (300 mg, crude, TFA) as a yellow oil. LCMS (ES⁺): m/z 439.2 [M+H]⁺.

Synthesis of cis-N-(1-((1-(3-(2,6-dioxopiperidin-3-yl)-1-methyl-1H-indazol-6-yl)piperidin-4-yl)methyl)piperidin-4-yl)-3-((5-fluoro-4-(3-(2-oxopyridin-1(2H)-yl)phenyl)pyrimidin-2-yl)amino)cyclohexane-1-carboxamide (Compound 332). To a solution of cis-3-((5-fluoro-4-(3-(2-oxopyridin-1(2H)-yl)phenyl)pyrimidin-2-yl)amino)cyclohexane-1-carboxylic acid (279 mg, 684 μmol, 1.00 eq) in DMF (6 mL) were added HATU (390 mg, 1.03 mmol, 1.50 eq) and DIEA (265 mg, 2.05 mmol, 357 μL, 3.00 eq) at 25 °C. Then 3-(6-(4-((4-(12-azaneyl)piperidin-1-yl)methyl)piperidin-1-yl)-1-methyl-1H-indazol-3-yl)piperidine-2,6-dione (300 mg, 684 μmol, 1.00 eq) was added at 25 °C. The resulting mixture was stirred at 25 °C for 2 h. LCMS showed that 3-(6-(4-((4-(12-azaneyl)piperidin-1-yl)methyl)piperidin-1-yl)-1-methyl-1H-indazol-3-yl)piperidine-2,6-dione was consumed completely and the desired mass was detected. The reaction was filtered and the filtrate was concentrated under reduced pressure to give a residue. The residue was purified by prep-HPLC (column: Phenomenex Luna C18 75*30 mm*3 um; mobile phase: [H₂O (0.2% FA)-ACN]; gradient: 15%-45% B over 8.0 min) to give the title compound (Compound 332) (7.20 mg, 8.01 μmol, 1.17% yield, 92.3% purity) as a yellow solid, LCMS (ES⁺): m/z 829.4 [M+H]⁺

### Example 329. Synthesis of cis-trans-N-(4-((4-(3-(2,6-dioxopiperidin-3-yl)-2-oxo-2,3-dihydrobenzo[d]oxazol-6-yl)piperidin-1-yl)methyl)cyclohexyl)-3-((5-fluoro-4-(3-(2-oxopyridin-1(2H)-yl)phenyl)pyrimidin-2-yl)amino)cyclohexane-1-carboxamide (Compound 306)

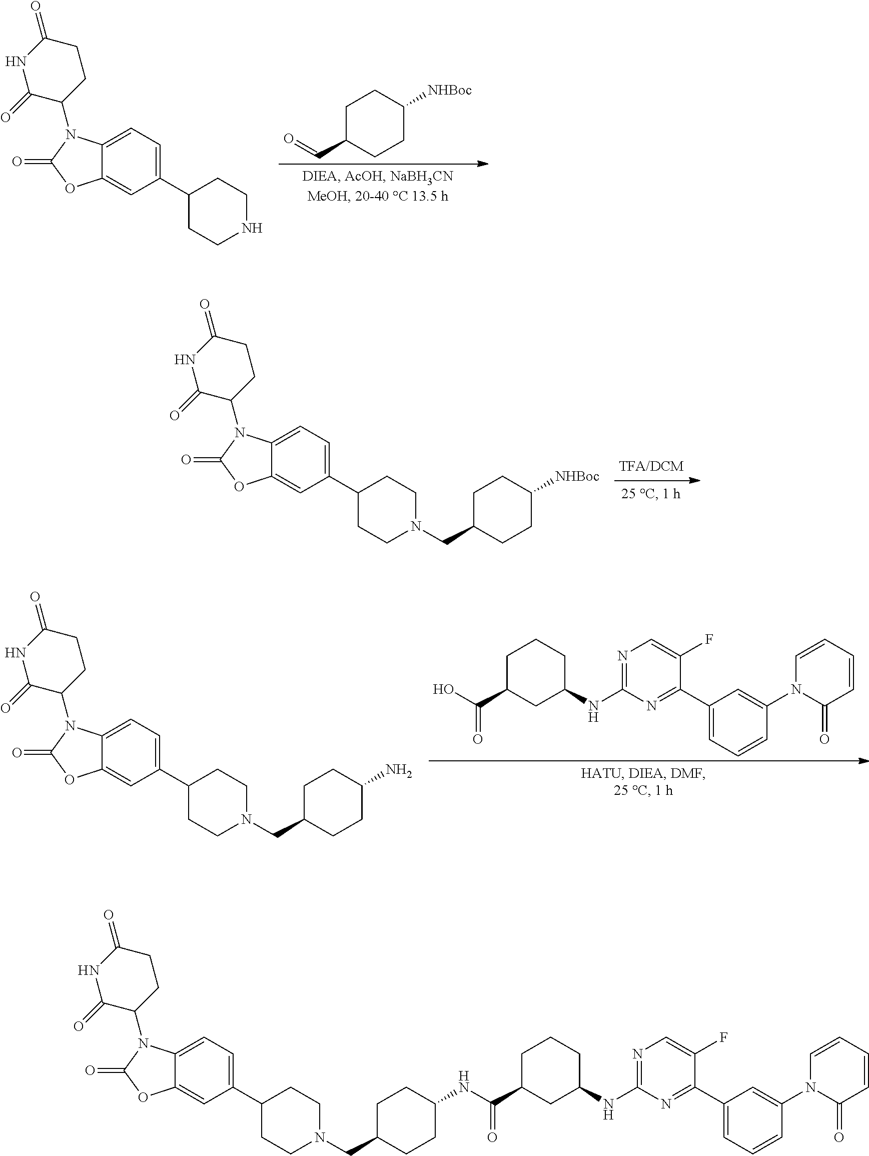 [SMILES: O=C1CCC(n2c(=O)oc3cc(C4CCNCC4)ccc32)C(=O)N1 ; CC(C)(C)OC(=O)N[C@H]1CC[C@H](C=O)CC1 ; CC(C)(C)OC(=O)N[C@H]1CC[C@H](CN2CCC(c3ccc4c(c3)oc(=O)n4C3CCC(=O)NC3=O)CC2)CC1 ; N[C@H]1CC[C@H](CN2CCC(c3ccc4c(c3)oc(=O)n4C3CCC(=O)NC3=O)CC2)CC1 ; O=C(O)[C@H]1CCC[C@@H](Nc2ncc(F)c(-c3cccc(-n4ccccc4=O)c3)n2)C1 ; O=C1CCC(n2c(=O)oc3cc(C4CCN(C[C@H]5CC[C@H](NC(=O)[C@H]6CCC[C@@H](Nc7ncc(F)c(-c8cccc(-n9ccccc9=O)c8)n7)C6)CC5)CC4)ccc32)C(=O)N1]

Synthesis of trans-tert-butyl (4-((4-(3-(2,6-dioxopiperidin-3-yl)-2-oxo-2,3-dihydrobenzo[d]oxazol-6-yl)piperidin-1-yl)methyl)cyclohexyl)carbamate. To a solution of 3-(2-oxo-6-(piperidin-4-yl)benzo[d]oxazol-3 (2H)-yl)piperidine-2,6-dione (500 mg, 1.13 mmol, 1.00 eq, TFA) in MeOH (10 mL) was added DIEA at 20 °C to adjust pH to 8. The mixture was stirred at 20 °C for 1 h. Then to the mixture were added trans-tert-butyl (4-formylcyclohexyl)carbamate (308 mg, 1.35 mmol, 1.20 eq) and AcOH (339 mg, 5.64 mmol, 323 μL, 5.00 eq) at 20 °C, pH was 5 now. The mixture was stirred at 40 °C for 0.5 h. Then to the mixture was added NaBH₃CN (141 mg, 2.26 mmol, 2.00 eq) at 20 °C. The resulting mixture was stirred at 40 °C for 12 h. LCMS showed 3-(2-oxo-6-(piperidin-4-yl)benzo[d]oxazol-3 (2H)-yl)piperidine-2,6-dione was consumed completely and the desired mass was detected. The mixture was concentrated under reduced pressure to give a residue. The residue was diluted with H₂O (20 mL) and then extracted with EtOAc (15 mL*3). The combined organic layers were dried over Na₂SO₄, filtered and the filtrate was concentrated under reduced pressure to give a residue. The residue was purified by prep-TLC (SiO₂, EtOAc/MeOH=20/1) to give the title compound (240 mg, 444 μmol, 39.4% yield) as a yellow solid. LCMS (ES⁺): m/z 541.4 [M+H]⁺.

Synthesis of trans-3-(6-(1-((4-aminocyclohexyl)methyl)piperidin-4-yl)-2-oxobenzo[d]oxazol-3 (2H)-yl)piperidine-2,6-dione. A solution of trans-tert-butyl (4-((4-(3-(2,6-dioxopiperidin-3-yl)-2-oxo-2,3-dihydrobenzo[d]oxazol-6-yl)piperidin-1-yl)methyl)cyclohexyl)carbamate (220 mg, 407 μmol, 1.00 eq) in DCM (1.5 mL) and TFA (0.5 mL) was stirred at 25 °C for 1 h. LCMS showed trans-tert-butyl (4-((4-(3-(2,6-dioxopiperidin-3-yl)-2-oxo-2,3-dihydrobenzo[d]oxazol-6-yl)piperidin-1-yl)methyl)cyclohexyl)carbamate was consumed completely and the desired mass was detected. The mixture was concentrated under reduced pressure to give the title compound (220 mg, crude, TFA) as a yellow oil. LCMS (ES⁺): m/z 441.3 [M+H]⁺.

Synthesis of cis-trans-N-(4-((4-(3-(2,6-dioxopiperidin-3-yl)-2-oxo-2,3-dihydrobenzo[d]oxazol-6-yl)piperidin-1-yl)methyl)cyclohexyl)-3-((5-fluoro-4-(3-(2-oxopyridin-1(2H)-yl)phenyl)pyrimidin-2-yl)amino)cyclohexane-1-carboxamide (Compound 306). To a solution of cis-3-((5-fluoro-4-(3-(2-oxopyridin-1(2H)-yl)phenyl)pyrimidin-2-yl)amino)cyclohexane-1-carboxylic acid (81.0 mg, 198 μmol, 0.500 eq) in DMF (2 mL) were added HATU (229 mg, 595 μmol, 1.50 eq) and DIEA (513 mg, 198 mmol, 346 μL, 5.00 eq) at 25 °C, then trans-3-(6-(1-((4-aminocyclohexyl)methyl)piperidin-4-yl)-2-oxobenzo[d]oxazol-3 (2H)-yl)piperidine-2,6-dione (220 mg, 397 μmol, 1.00 eq, TFA) was added to the mixture at 25 °C, the mixture was stirred at 25 °C for 1 h. LCMS showed trans-3-(6-(1-((4-aminocyclohexyl)methyl)piperidin-4-yl)-2-oxobenzo[d]oxazol-3 (2H)-yl)piperidine-2,6-dione was consumed completely and the desired mass was detected. The mixture was diluted with H₂O (5 mL) and then extracted with EtOAc (3 mL*3). The combined organic layers were dried over Na₂SO₄, filtered and the filtrate was concentrated under reduced pressure to give a residue. The residue was purified by prep-HPLC (column: Phenomenex Luna C18 100*30 mm*3 um; mobile phase: [H₂O (0.2% FA)-ACN]; gradient: 10%-50% B over 8.0 min) to give the title compound (Compound 306) (90.6 mg, 99.2 μmol, 24.7% yield, 91.0% purity) as a yellow solid. LCMS (ES⁺): m/z 831.1 [M+H]⁺

### Example 330. Synthesis of cis-trans-N-(4-((4-(3-(2,6-dioxopiperidin-3-yl)-1-methyl-1H-indazol-6-yl)piperidin-1-yl)methyl)cyclohexyl)-3-((5-fluoro-4-(3-(2-oxopyridin-1(2H)-yl)phenyl)pyrimidin-2-yl)amino)cyclohexane-1-carboxamide (Compound 307)

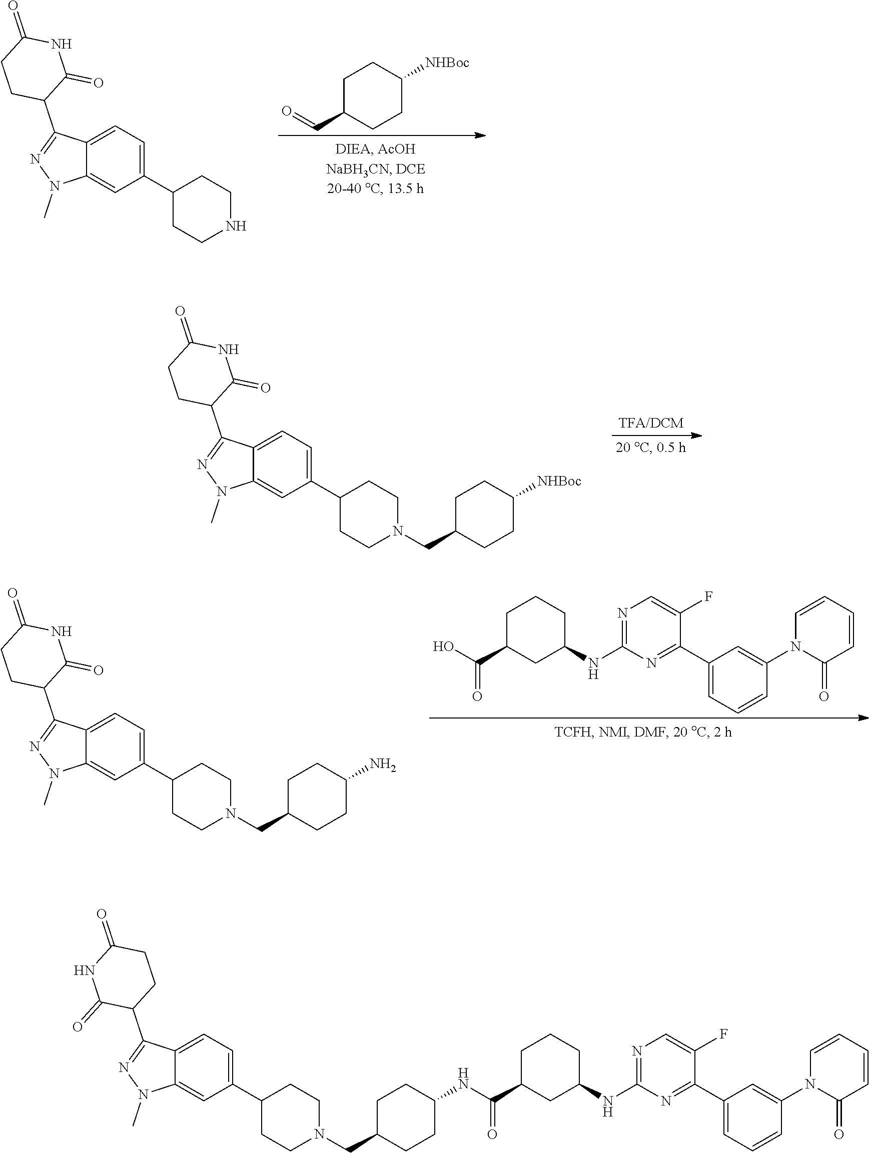 [SMILES: Cn1nc(C2CCC(=O)NC2=O)c2ccc(C3CCNCC3)cc21 ; CC(C)(C)OC(=O)N[C@H]1CC[C@H](C=O)CC1 ; Cn1nc(C2CCC(=O)NC2=O)c2ccc(C3CCN(C[C@H]4CC[C@H](NC(=O)OC(C)(C)C)CC4)CC3)cc21 ; Cn1nc(C2CCC(=O)NC2=O)c2ccc(C3CCN(C[C@H]4CC[C@H](N)CC4)CC3)cc21 ; O=C(O)[C@H]1CCC[C@@H](Nc2ncc(F)c(-c3cccc(-n4ccccc4=O)c3)n2)C1 ; Cn1nc(C2CCC(=O)NC2=O)c2ccc(C3CCN(C[C@H]4CC[C@H](NC(=O)[C@H]5CCC[C@@H](Nc6ncc(F)c(-c7cccc(-n8ccccc8=O)c7)n6)C5)CC4)CC3)cc21]

Synthesis of trans-tert-butyl (4-((4-(3-(2,6-dioxopiperidin-3-yl)-1-methyl-1H-indazol-6-yl)piperidin-1-yl)methyl)cyclohexyl)carbamate. To a solution of 3-(1-methyl-6-(piperidin-4-yl)-1H-indazol-3-yl)piperidine-2,6-dione (800 mg, 1.82 mmol, 1.00 eq, TFA) in DCE (15 mL) was added DIEA to adjust pH to 8 at 20 °C. The mixture was stirred at 20 °C for 1 h. Then CH₃COOH (545 mg, 9.08 mmol, 520 μL, 5.00 eq) and trans-tert-butyl (4-formylcyclohexyl) carbamate (826 mg, 3.63 mmol, 2.00 eq) was added to the mixture at 20 °C and the mixture was stirred at 40 °C for 0.5 h, pH was 5 now. Then NaBH₃CN (799 mg, 12.7 mmol, 7.00 eq) was added to the mixture at 20 °C. The resulting mixture was stirred at 40 °C for 12 h. LCMS showed 3-(1-methyl-6-(piperidin-4-yl)-1H-indazol-3-yl)piperidine-2,6-dione was consumed and the desired target MS was detected. H₂O (15 mL) was added to the solution, the mixture was extracted with DCM (15 mL*3), then the combined organic layers were dried over Na₂SO₄, then concentrated under reduced pressure to give a residue. The residue was purified by MPLC (SiO₂, EtOAc/MeOH=1/0 to 1/1) to give the title compound (800 mg, 1.49 mmol, 81.9% yield) as a yellow oil. LCMS (ES⁺): m/z 538.4 [M+H]⁺.

Synthesis of trans-3-(6-(1-((4-aminocyclohexyl)methyl)piperidin-4-yl)-1-methyl-1H-indazol-3-yl)piperidine-2,6-dione. A solution of trans-tert-butyl (4-((4-(3-(2,6-dioxopiperidin-3-yl)-1-methyl-1H-indazol-6-yl)piperidin-1-yl)methyl)cyclohexyl)carbamate (200 mg, 372 μmol, 1.00 eq) in TFA (1 mL) and DCM (3 mL) was stirred at 20 °C for 0.5 h. LCMS showed trans-tert-butyl (4-((4-(3-(2,6-dioxopiperidin-3-yl)-1-methyl-1H-indazol-6-yl)piperidin-1-yl)methyl)cyclohexyl)carbamate was consumed and the desired target MS was detected. The mixture was concentrated under reduced pressure to give the title compound (200 mg, crude, TFA) as a yellow oil. LCMS (ES⁺): m/z 438.3 [M+H]⁺.

Synthesis of cis-trans-N-(4-((4-(3-(2,6-dioxopiperidin-3-yl)-1-methyl-1H-indazol-6-yl)piperidin-1-yl)methyl)cyclohexyl)-3-((5-fluoro-4-(3-(2-oxopyridin-1(2H)-yl)phenyl)pyrimidin-2-yl)amino)cyclohexane-1-carboxamide (Compound 307). To a solution of cis-3-((5-fluoro-4-(3-(2-oxopyridin-1(2H)-yl)phenyl)pyrimidin-2-yl)amino)cyclohexane-1-carboxylic acid (148 mg, 363 μmol, 1.00 eq) in DMF (3 mL) were added TCFH (112 mg, 399 μmol, 1.10 eq), NMI (298 mg, 3.62 mmol, 289 μL, 10 eq) and trans-3-(6-(1-((4-aminocyclohexyl)methyl)piperidin-4-yl)-1-methyl-1H-indazol-3-yl)piperidine-2,6-dione (200 mg, 363 μmol, 1.00 eq, TFA) at 20 °C, then the mixture was stirred at 20 °C for 2 h. LCMS showed trans-3-(6-(1-((4-aminocyclohexyl)methyl)piperidin-4-yl)-1-methyl-1H-indazol-3-yl)piperidine-2,6-dione was consumed and the desired target MS was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 10%-45% B over 8.0 min) to give the title compound (Compound 307) (20.7 mg, 23.3 μmol, 6.42% yield, 93.1% purity) as a white solid. LCMS (ES⁺): m/z 828.4 [M+H]⁺

### Example 331. Synthesis of cis-N-((1′-(3-(2,6-dioxopiperidin-3-yl)-1-methyl-1H-indazol-6-yl)-[1,4′-bipiperidin]-4-yl)methyl)-3-((5-fluoro-4-(3-(2-oxopyridin-1(2H)-yl)phenyl)pyrimidin-2-yl)amino)cyclohexane-1-carboxamide (Compound 333)

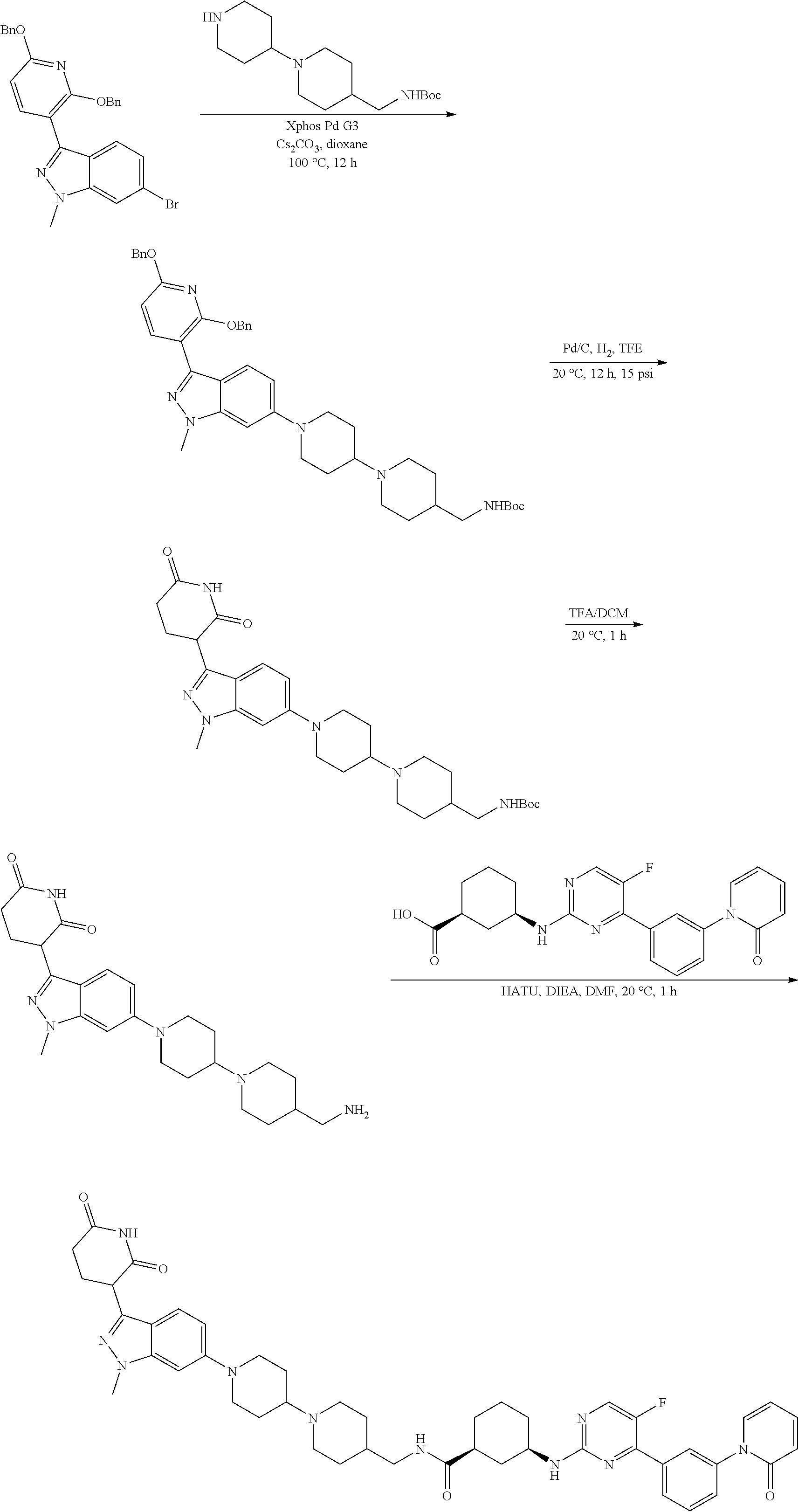

Synthesis of tert-butyl N-[[1-[1-[3-(2,6-dibenzyloxy-3-pyridyl)-1-methyl-indazol-6-yl]-4-piperidyl]-4-piperidyl]methyl]carbamate. To a stirred solution of 6-bromo-3-(2,6-dibenzyloxy-3-pyridyl)-1-methyl-indazole (1.55 g, 3.09 mmol, 1.00 eq) and tert-butyl N-[[1-(4-piperidyl)-4-piperidyl]methyl]carbamate (920 mg, 3.09 mmol, 1.00 eq) in dioxane (50 mL) were added Cs₂CO₃ (2.02 g, 6.19 mmol, 2.00 eq) and XPhos Pd G3 (262 mg, 309 μmol, 0.100 eq) at 20 °C under N₂. The resulting mixture was stirred at 100 °C for 12 h under N₂. LCMS showed 6-bromo-3-(2,6-dibenzyloxy-3-pyridyl)-1-methyl-indazole was consumed completely and the desired mass was detected. The mixture was diluted with H₂O (50 mL) and then extracted with EtOAc (40 mL*3). The combined organic layers were dried over Na₂SO₄, filtered and the filtrate was concentrated under reduced pressure to give a residue. The residue was purified by MPLC (SiO₂, PE/EtOAc=0/1) to give the title compound (550 mg, 767 μmol, 24.8% yield) as a yellow solid. LCMS (ES⁺): m/z 717.4 [M+H]⁺.

Synthesis of tert-butyl N-[[1-[1-[3-(2,6-dioxo-3-piperidyl)-1-methyl-indazol-6-yl]-4-piperidyl]-4-piperidyl]methyl]carbamate. To a stirred solution of tert-butyl N-[[1-[1-[3-(2,6-dibenzyloxy-3-pyridyl)-1-methyl-indazol-6-yl]-4-piperidyl]-4-piperidyl]methyl]carbamate (500 mg, 697 μmol, 1.00 eq) in TFE (10 mL) was added 10% Pd/C (500 mg, 50% purity) at 20 °C under N₂. The mixture was degassed and purged with H₂ for three times and the resulting mixture was stirred at 20 °C for 12 h under H₂ (15 psi). LCMS showed tert-butyl N-[[1-[1-[3-(2,6-dibenzyloxy-3-pyridyl)-1-methyl-indazol-6-yl]-4-piperidyl]-4-piperidyl]methyl]carbamate was consumed completely and the desired mass was detected. The mixture was filtered and the filtrate was concentrated under reduced pressure to give the title compound (300 mg, crude) as a yellow oil. LCMS (ES⁺): m/z 539.3 [M+H]⁺.

Synthesis of 3-[6-[4-[4-(aminomethyl)-1-piperidyl]-1-piperidyl]-1-methyl-indazol-3-yl]piperidine-2,6-dione. To a solution of tert-butyl N-[[1-[1-[3-(2,6-dioxo-3-piperidyl)-1-methyl-indazol-6-yl]-4-piperidyl]-4-piperidyl]methyl]carbamate (300 mg, 557 μmol, 1.00 eq) in DCM (4 mL) was added TFA (1 mL) at 20 °C, the mixture was stirred at 20 °C for 1 h. LCMS showed that tert-butyl N-[[1-[1-[3-(2,6-dioxo-3-piperidyl)-1-methyl-indazol-6-yl]-4-piperidyl]-4-piperidyl]methyl]carbamate was consumed completely and the desired mass was detected. The mixture was concentrated under reduced pressure to give the title compound (150 mg, crude, TFA) as a black oil liquid. LCMS (ES⁺): m/z 439.2 [M+H]⁺.

Synthesis of cis-N-((1′-(3-(2,6-dioxopiperidin-3-yl)-1-methyl-1H-indazol-6-yl)-[1,4′-bipiperidin]-4-yl)methyl)-3-((5-fluoro-4-(3-(2-oxopyridin-1(2H)-yl)phenyl)pyrimidin-2-yl)amino)cyclohexane-1-carboxamide (Compound 333). To a solution of cis-3-((5-fluoro-4-(3-(2-oxopyridin-1(2H)-yl)phenyl)pyrimidin-2-yl)amino)cyclohexane-1-carboxylic acid (100 mg, 245 μmol, 1.00 eq) in DMF (4 mL) was added HATU (140 mg, 367 μmol, 1.50 eq) and DIEA (158 mg, 1.23 mmol, 213 μL, 5.00 eq) at 20 °C, the mixture was stirred at 20 °C for 0.5 h. Then 3-[6-[4-[4-(aminomethyl)-1-piperidyl]-1-piperidyl]-1-methyl-indazol-3-yl]piperidine-2,6-dione (135 mg, 245 μmol, 1.00 eq, TFA) was added at 20 °C, the mixture was stirred at 20 °C for 0.5 h. LCMS showed that 3-[6-[4-[4-(aminomethyl)-1-piperidyl]-1-piperidyl]-1-methyl-indazol-3-yl]piperidine-2,6-dione was consumed completely and the desired mass was detected. To the mixture was added H₂O (5 mL) and the mixture was extracted with EtOAc (5 mL*3). The combined organic layers were washed with brine (5 mL*3), dried over Na₂SO₄, filtered and the filtrate was concentrated under reduced pressure to give a residue. The residue was purified by prep-HPLC (column: 3_Phenomenex Luna C18 75*30 mm*3 um; mobile phase: [H₂O (0.1% TFA)-ACN]; gradient: 20%-50% B over 8.0 min) to give the title compound (Compound 333) (2.00 mg, 2.37 μmol, 0.968% yield, 98.3% purity) as a white solid. LCMS (ES⁺): m/z 829.4 [M+H]⁺

### Example 332. Synthesis of cis-trans-N-((4-(4-(3-(2,6-dioxopiperidin-3-yl)-2-oxo-2,3-dihydrobenzo[d]oxazol-6-yl)piperidin-1-yl)cyclohexyl)methyl)-3-((5-fluoro-4-(3-(2-oxopyridin-1(2H)-yl)phenyl)pyrimidin-2-yl)amino)cyclohexane-1-carboxamide (Compound 349)

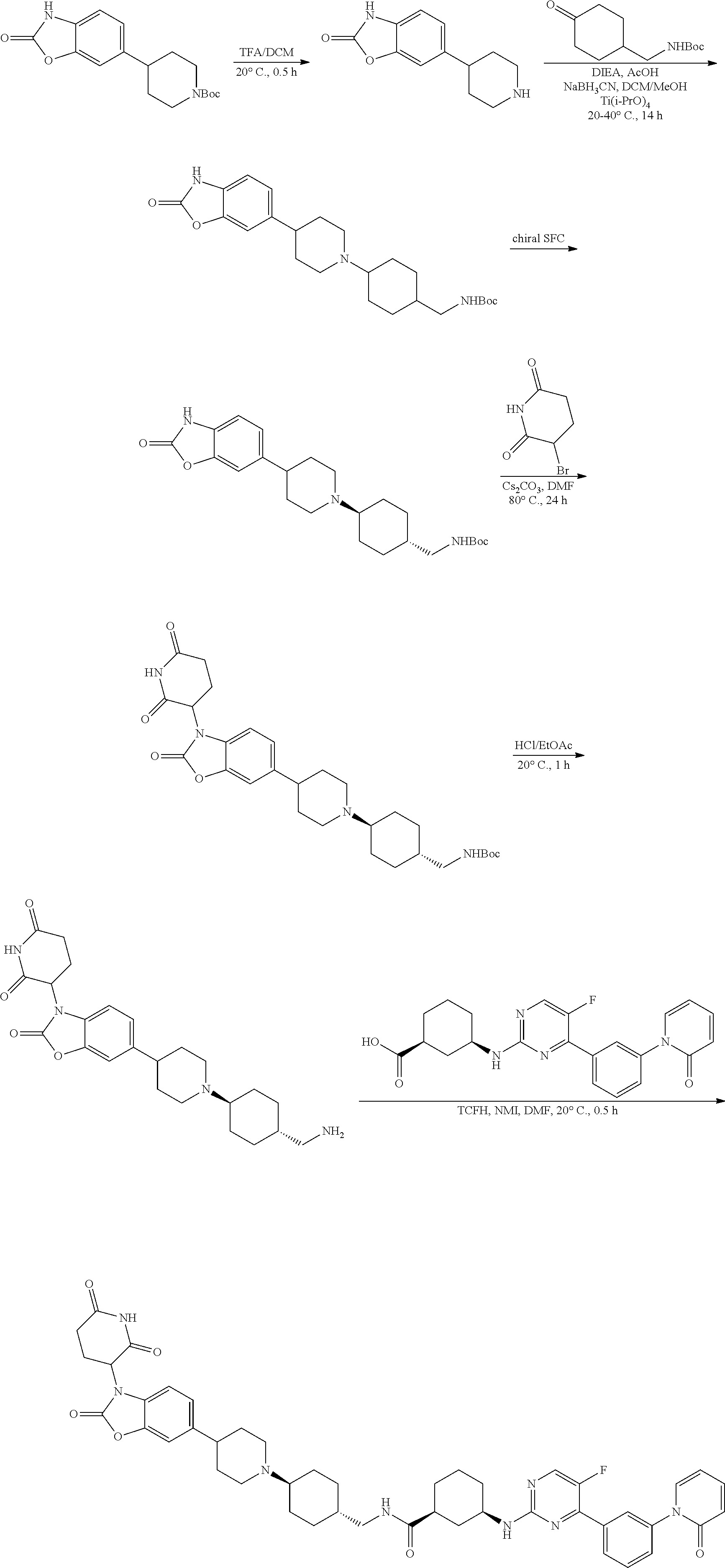

Synthesis of 6-(piperidin-4-yl)benzo[d]oxazol-2(3H)-one. A solution of tert-butyl 4-(2-oxo-2,3-dihydrobenzo[d]oxazol-6-yl)piperidine-1-carboxylate (5.30 g, 16.7 mmol, 1.00 eq) in TFA (15 mL) and DCM (45 mL) was stirred at 20 °C for 0.5 h. LCMS showed tert-butyl 4-(2-oxo-2,3-dihydrobenzo[d]oxazol-6-yl)piperidine-1-carboxylate was consumed and the desired target MS was detected. The mixture was concentrated under reduced pressure to give the title compound (5.30 g, crude, TFA) as a yellow oil. LCMS (ES⁺): m/z 219.2 [M+H]⁺

Synthesis of tert-butyl ((4-(4-(2-oxo-2,3-dihydrobenzo[d]oxazol-6-yl)piperidin-1-yl)cyclohexyl)methyl)carbamate. To a solution of 6-(piperidin-4-yl)benzo[d]oxazol-2(3H)-one. (3.00 g, 9.03 mmol, 1.00 eq, TFA) in DCM (40 mL) and MeOH (40 mL) was added DIEA to adjust pH to 8 at 20 °C. The mixture was stirred at 20 °C for 1 h. Then CH₃COOH (2.71 g, 45.1 mmol, 2.58 mL, 5.00 eq), Ti(i-PrO)₄ (2.57 g, 9.03 mmol, 2.66 mL, 1.00 eq) and tert-butyl ((4-oxocyclohexyl)methyl)carbamate (10.3 g, 45.1 mmol, 5.00 eq) were added to the mixture at 20 °C, pH was 5 now. The mixture was stirred at 40 °C for 1 h. Then to the reaction mixture was added NaBH₃CN (2.84 g, 45.1 mmol, 5.00 eq) at 20 °C. The resulting mixture was stirred at 40 °C for 12 h. LCMS showed 6-(piperidin-4-yl)benzo[d]oxazol-2(3H)-one was consumed and the desired target MS was detected. The reaction was concentrated under reduced pressure to give a residue. Then H₂O (40 mL) was added to the residue, the mixture was extracted with EtOAc (40 mL*3), then the combined organic layers were dried over Na₂SO₄, filtered and the filtrate was concentrated under reduced pressure to give a residue. The residue was purified by prep-HPLC (column: Phenomenex luna C18 (250*70 mm, 15 um); mobile phase: [H₂O (0.2% FA)-ACN]; gradient: 30%-60% B over 20.0 min) to give the title compound (4.00 g, crude) as a yellow oil. LCMS (ES⁺): m/z 430.3 [M+H]⁺

Synthesis of trans-tert-butyl ((4-(4-(2-oxo-2,3-dihydrobenzo[d]oxazol-6-yl)piperidin-1-yl)cyclohexyl)methyl)carbamate. tert-butyl ((4-(4-(2-oxo-2,3-dihydrobenzo[d]oxazol-6-yl)piperidin-1-yl)cyclohexyl)methyl)carbamate (4.00 g, crude) was separated by chiral SFC (column: DAICEL CHIRALPAK IC (250 mm*30 mm, 10 um); mobile phase: [CO₂-EtOH (0.1% NH₃H₂O)]; B %: 80%, isocratic elution mode) to give the title compound (100 mg, 233 μmol, 2.58% yield) as a yellow oil. LCMS (ES⁺): m/z 430.3 [M+H]⁺

Synthesis of trans-tert-butyl ((4-(4-(3-(2,6-dioxopiperidin-3-yl)-2-oxo-2,3-dihydrobenzo[d]oxazol-6-yl)piperidin-1-yl)cyclohexyl)methyl)carbamate. To a solution of trans-tert-butyl ((4-(4-(2-oxo-2,3-dihydrobenzo[d]oxazol-6-yl)piperidin-1-yl)cyclohexyl)methyl)carbamate (100 mg, 233 μmol, 1.00 eq) and 3-bromopiperidine-2,6-dione (134 mg, 698 μmol, 3.00 eq) in DMF (6 mL) was added Cs₂CO₃ (152 mg, 466 μmol, 2.00 eq) at 20 °C, the mixture was stirred at 80 °C for 12 h. LCMS showed trans-tert-butyl ((4-(4-(2-oxo-2,3-dihydrobenzo[d]oxazol-6-yl)piperidin-1-yl)cyclohexyl)methyl)carbamate remained and the desired target MS was detected. Then 3-bromopiperidine-2,6-dione (89.4 mg, 466 μmol, 2.00 eq) and Cs₂CO₃ (75.9 mg, 233 μmol, 1.00 eq) were added to the mixture, the mixture was stirred at 80 °C for another 12 h, LCMS showed trans-tert-butyl ((4-(4-(2-oxo-2,3-dihydrobenzo[d]oxazol-6-yl)piperidin-1-yl)cyclohexyl)methyl)carbamate was consumed and the desired target MS was detected. H₂O (8 mL) was added to the solution, the mixture was extracted with EtOAc (8 mL*3), then the combined organic layers were washed with brine (5 mL*3), dried over Na₂SO₄, filtered and the filtrate was concentrated under reduced pressure to give the title compound (220 mg, crude) as a yellow oil. LCMS (ES⁺): m/z 541.3 [M+H]⁺

Synthesis of trans-3-(6-(1-(-4-(aminomethyl)cyclohexyl)piperidin-4-yl)-2-oxobenzo[d]oxazol-3 (2H)-yl)piperidine-2,6-dione. A solution of trans-tert-butyl ((-4-(4-(3-(2,6-dioxopiperidin-3-yl)-2-oxo-2,3-dihydrobenzo[d]oxazol-6-yl)piperidin-1-yl)cyclohexyl)methyl)carbamate (150 mg, 277 μmol, 1.00 eq) in HCl/EtOAc (4 M, 3 mL) was stirred at 20 °C for 1 h. LCMS showed trans-tert-butyl ((-4-(4-(3-(2,6-dioxopiperidin-3-yl)-2-oxo-2,3-dihydrobenzo[d]oxazol-6-yl)piperidin-1-yl)cyclohexyl)methyl)carbamate was consumed and the desired target MS was detected. The mixture was concentrated under reduced pressure to give the title compound (150 mg, crude, HCl) as a yellow oil. LCMS (ES⁺): m/z 441.2 [M+H]⁺

Synthesis of cis-trans-N-((4-(4-(3-(2,6-dioxopiperidin-3-yl)-2-oxo-2,3-dihydrobenzo[d]oxazol-6-yl)piperidin-1-yl)cyclohexyl)methyl)-3-((5-fluoro-4-(3-(2-oxopyridin-1(2H)-yl)phenyl)pyrimidin-2-yl)amino)cyclohexane-1-carboxamide (Compound 349). To a solution of cis-3-((5-fluoro-4-(3-(2-oxopyridin-1(2H)-yl)phenyl)pyrimidin-2-yl)amino)cyclohexane-1-carboxylic acid (22.1 mg, 54.1 μmol, 0.200 eq) in DMF (1.5 mL) were added NMI (68.8 mg, 839 μmol, 66.8 μL, 3.10 eq) and TCFH (83.5 mg, 298 μmol, 1.10 eq) at 20 °C, then trans-3-(6-(1-(-4-(aminomethyl)cyclohexyl)piperidin-4-yl)-2-oxobenzo[d]oxazol-3 (2H)-yl)piperidine-2,6-dione (150 mg, 270 μmol, 1.00 eq, TFA) was added to the mixture, the mixture was stirred at 20 °C for 0.5 h. LCMS showed trans-3-(6-(1-(-4-(aminomethyl)cyclohexyl)piperidin-4-yl)-2-oxobenzo[d]oxazol-3 (2H)-yl)piperidine-2,6-dione was consumed and the desired target MS was detected. The mixture was concentrated under reduced pressure to give a residue. The residue was purified by prep-HPLC (column: Phenomenex Luna C18 80*30 mm*3 um; mobile phase: [H₂O (0.1% TFA)-ACN]; gradient: 10%-40% B over 8.0 min) to give the title compound (Compound 349) (5.00 mg, 5.89 μmol, 2.18% yield, 98.0% purity) as a pink solid. LCMS (ES⁺): m/z 831.4 [M+H]⁺

### Example 333. Synthesis of cis-trans-N-((4-(4-(3-(2,6-dioxopiperidin-3-yl)-1-methyl-1H-indazol-6-yl)piperidin-1-yl)cyclohexyl)methyl)-3-((5-fluoro-4-(3-(2-oxopyridin-1(2H)-yl)phenyl)pyrimidin-2-yl)amino)cyclohexane-1-carboxamide (Compound 334)

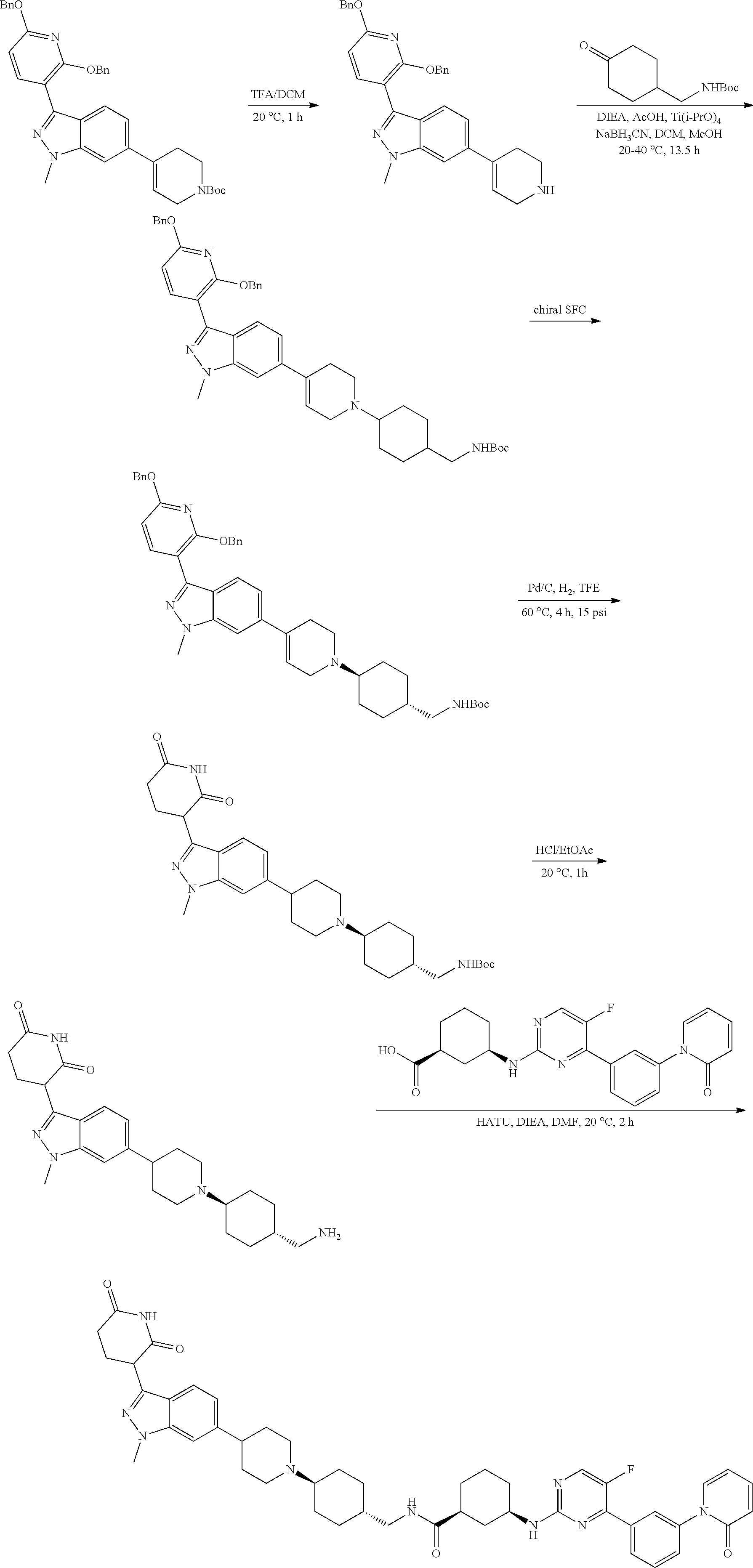

Synthesis of 3-(2,6-bis(benzyloxy)pyridin-3-yl)-1-methyl-6-(1,2,3,6-tetrahydropyridin-4-yl)-1H-indazole. A solution of tert-butyl 4-(3-(2,6-bis(benzyloxy)pyridin-3-yl)-1-methyl-1H-indazol-6-yl)-3,6-dihydropyridine-1(2H)-carboxylate (3.02 g, 5.01 mmol, 1.00 eq) in TFA (3 mL) and DCM (30 mL) was stirred at 20 °C for 1 h. LCMS showed tert-butyl 4-(3-(2,6-bis(benzyloxy)pyridin-3-yl)-1-methyl-1H-indazol-6-yl)-3,6-dihydropyridine-1(2H)-carboxylate was consumed completely and the desired target MS was detected. The mixture was concentrated under reduced pressure to give the title compound (3.02 g, crude, TFA) as a yellow oil. LCMS (ES⁺): m/z 503.2 [M+H]⁺

Synthesis of tert-butyl ((4-(4-(3-(2,6-bis(benzyloxy)pyridin-3-yl)-1-methyl-1H-indazol-6-yl)-3,6-dihydropyridin-1(2H)-yl)cyclohexyl)methyl)carbamate. To a solution of 3-(2,6-bis(benzyloxy)pyridin-3-yl)-1-methyl-6-(1,2,3,6-tetrahydropyridin-4-yl)-1H-indazole (3.00 g, 4.87 mmol, 1.00 eq, TFA) in DCM (40 mL) and MeOH (40 mL) was added DIEA to adjust pH to 8 at 20 °C. The mixture was stirred at 20 °C for 1 h. To the mixture was added CH₃COOH (1.46 g, 24.33 mmol, 1.39 mL, 5.00 eq), Ti(i-PrO)₄ (1.38 g, 4.87 mmol, 1.44 mL, 1.00 eq) and tert-butyl ((4-oxocyclohexyl)methyl)carbamate (5.53 g, 24.3 mmol, 5.00 eq) at 40 °C, pH was 5 now. The mixture was stirred at 20 °C for 0.5 h. Then to the mixture was added NaBH₃CN (1.53 g, 24.3 mmol, 5.00 eq) at 20 °C. The resulting mixture was stirred at 40 °C for 12 h. LCMS showed 3-(2,6-bis(benzyloxy)pyridin-3-yl)-1-methyl-6-(1,2,3,6-tetrahydropyridin-4-yl)-1H-indazole was consumed, desired target MS was detected. The mixture was concentrated under reduced pressure to give the residue. To the residue was added H₂O (40 mL) and then the mixture was extracted with EtOAc (40 mL*3), the combined organic layers were dried over Na₂SO₄, filtered and the filtrate was concentrated under reduced pressure to give a residue. The residue was purified by prep-HPLC (column: Phenomenex luna C18 (250*70 mm, 15 um); mobile phase: [H₂O (0.2% FA)-ACN]; gradient: 60%-90% B over 20.0 min) to give the title compound (2.00 g, 280 mmol, 57.6% yield) as a yellow oil. LCMS (ES⁺): m/z 714.3 [M+H]⁺

Synthesis of trans-tert-butyl ((4-(4-(3-(2,6-bis(benzyloxy)pyridin-3-yl)-1-methyl-1H-indazol-6-yl)-3,6-dihydropyridin-1(2H)-yl)cyclohexyl)methyl)carbamate. tert-butyl ((4-(4-(3-(2,6-bis(benzyloxy)pyridin-3-yl)-1-methyl-1H-indazol-6-yl)-3,6-dihydropyridin-1(2H)-yl)cyclohexyl)methyl)carbamate (2.00 g, 280 mmol, 1.00 eq) was separated by chiral SFC (column: DAICEL CHIRALPAK IC (250 mm*30 mm, 10 um); mobile phase: [CO₂-MeOH:CAN=1:1 (0.1% NH₃H₂O)]; B %: 65%, isocratic elution mode) to give the title compound (800 mg, 1.12 mmol, 23.0% yield) as a yellow oil. LCMS (ES⁺): m/z 714.3 [M+H]⁺

Synthesis of trans-tert-butyl ((4-(4-(3-(2,6-dioxopiperidin-3-yl)-1-methyl-1H-indazol-6-yl)piperidin-1-yl)cyclohexyl)methyl)carbamate. To a solution of trans-tert-butyl ((4-(4-(3-(2,6-bis(benzyloxy)pyridin-3-yl)-1-methyl-1H-indazol-6-yl)-3,6-dihydropyridin-1(2H)-yl)cyclohexyl)methyl)carbamate (250 mg, 350 μmol, 1.00 eq) in TFE (10 mL) was added 10% Pd/C (500 mg, 50% purity) at 20 °C under N₂, the mixture was degassed and purged with H₂ for 3 times and the mixture was stirred at 60 °C for 4 h under H₂ (15 psi). LCMS showed trans-tert-butyl ((4-(4-(3-(2,6-bis(benzyloxy)pyridin-3-yl)-1-methyl-1H-indazol-6-yl)-3,6-dihydropyridin-1(2H)-yl)cyclohexyl)methyl)carbamate was consumed and the desired target MS was detected. The mixture was filtered and the filtrate was concentrated under reduced pressure to give the title compound (220 mg, crude) as a yellow oil. LCMS (ES⁺): m/z 538.4 [M+H]⁺

Synthesis of trans-3-(6-(1-(4-(aminomethyl)cyclohexyl)piperidin-4-yl)-1-methyl-1H-indazol-3-yl)piperidine-2,6-dione. A solution of trans-tert-butyl ((4-(4-(3-(2,6-dioxopiperidin-3-yl)-1-methyl-1H-indazol-6-yl)piperidin-1-yl)cyclohexyl)methyl)carbamate (150 mg, 279 μmol, 1.00 eq) in HCl/EtOAc (4 M, 3 mL) was stirred at 20 °C for 1 h. LCMS showed trans-tert-butyl ((4-(4-(3-(2,6-dioxopiperidin-3-yl)-1-methyl-1H-indazol-6-yl)piperidin-1-yl)cyclohexyl)methyl)carbamate was consumed and the desired target MS was detected. The mixture was concentrated under reduced pressure to give the title compound (150 mg, crude, HCl) as a yellow oil. LCMS (ES⁺): m/z 438.3 [M+H]⁺

Synthesis of cis-trans-N-((4-(4-(3-(2,6-dioxopiperidin-3-yl)-1-methyl-1H-indazol-6-yl)piperidin-1-yl)cyclohexyl)methyl)-3-((5-fluoro-4-(3-(2-oxopyridin-1(2H)-yl)phenyl)pyrimidin-2-yl)amino)cyclohexane-1-carboxamide (Compound 334). To a solution of cis-3-((5-fluoro-4-(3-(2-oxopyridin-1(2H)-yl)phenyl)pyrimidin-2-yl)amino)cyclohexane-1-carboxylic acid (90.5 mg, 222 μmol, 0.700 eq) and DIEA (204 mg, 1.58 mmol, 276 μL, 5.00 eq) in DMF (3 mL) were added HATU (180 mg, 475 μmol, 1.50 eq) and trans-3-(6-(1-((4-(aminomethyl)cyclohexyl)piperidin-4-yl)-1-methyl-1H-indazol-3-yl)piperidine-2,6-dione (150 mg, 316 μmol, 1.00 eq, HCl) at 20 °C, the mixture was stirred at 20 °C for 2 h. LCMS showed trans-3-(6-(1-((1r,4r)-4-(aminomethyl)cyclohexyl)piperidin-4-yl)-1-methyl-1H-indazol-3-yl)piperidine-2,6-dione was consumed and the desired target MS was detected. The mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 5%-35% B over 8.0 min) to give the title compound (Compound 334) (35.8 mg, 42.8 μmol, 13.5% yield, 99.1% purity) as a white solid. LCMS (ES⁺): m/z 828.4 [M+H]⁺

### Example 334. Synthesis of cis-3-(6-(4-(2-(1-(3-((5-fluoro-4-(3-(2-oxopyridin-1(2H)-yl)phenyl)pyrimidin-2-yl)amino)cyclohexane-1-carbonyl)piperidin-4-yl)ethyl)piperazin-1-yl)-2-oxobenzo[d]oxazol-3 (2H)-yl)piperidine-2,6-dione (Compound 342)

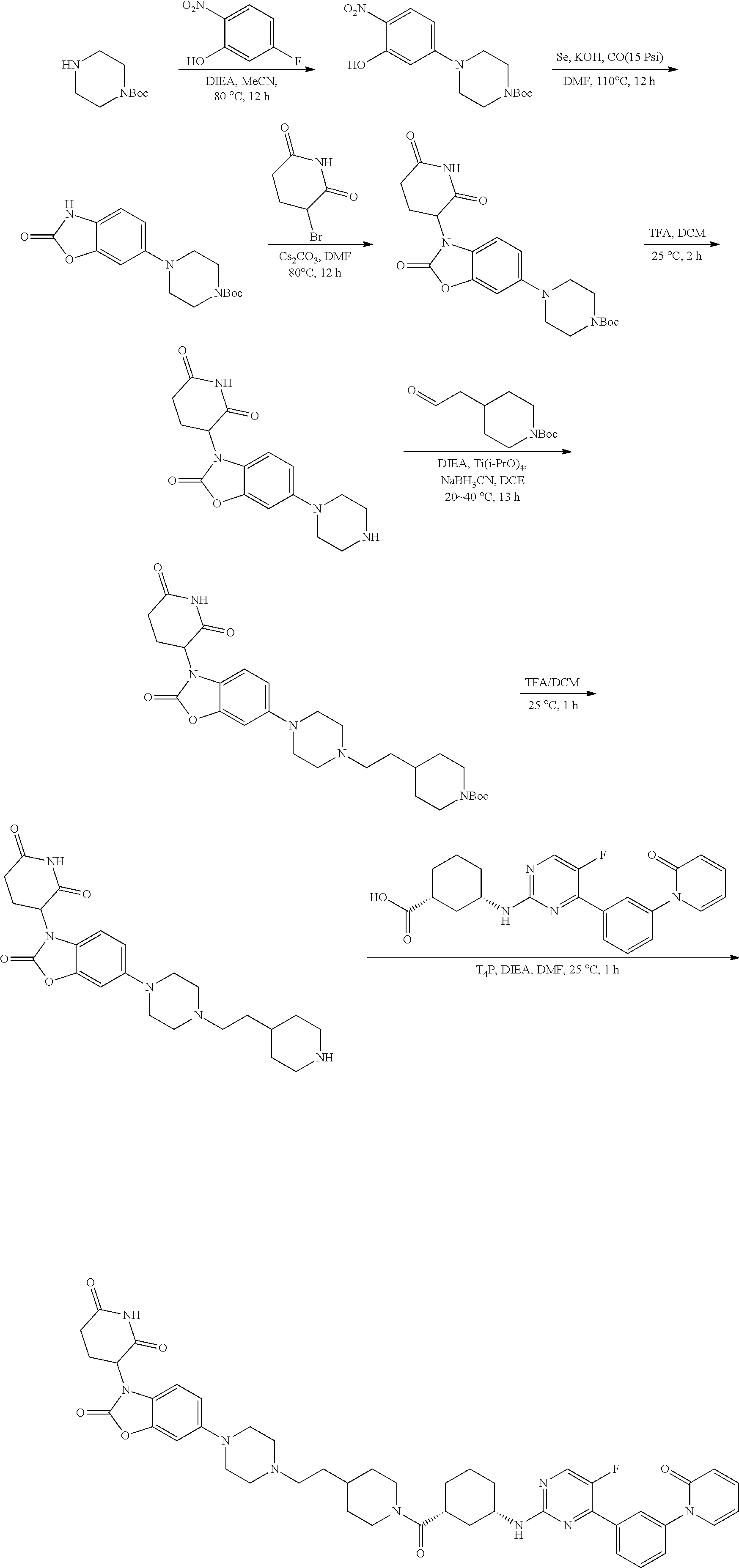

Synthesis of tert-butyl 4-(3-hydroxy-4-nitrophenyl)piperazine-1-carboxylate. To a solution of tert-butyl piperazine-1-carboxylate (8.00 g, 42.9 mmol, 1.00 eq) in MeCN (100 mL) was added 5-fluoro-2-nitro-phenol (6.75 g, 42.9 mmol, 1.00 eq) and DIEA (16.6 g, 128 mmol, 22.44 mL, 3.00 eq) at 25 °C. The mixture was stirred at 80 °C for 12 h. LC-MS showed desired mass was detected. The reaction mixture was concentrated under reduced pressure. The residue was purified by column chromatography (SiO₂, Petroleum ether:Ethyl acetate=1:0 to 3:1) to give the title compound (6.66 g, 20.4 mmol, 47.5% yield, 99.1% purity) as a yellow solid. LCMS (ES⁺): m/z 224.3 [M+H−100]⁺.

Synthesis of tert-butyl 4-(2-oxo-2,3-dihydrobenzo[d]oxazol-6-yl)piperazine-1-carboxylate. The reaction was set up in two batches. To a solution of tert-butyl 4-(3-hydroxy-4-nitrophenyl)piperazine-1-carboxylate (2.00 g, 6.19 mmol, 1.00 eq) in DMF (20 mL) was added Se (500 mg, 6.19 mmol, 1.00 eq) and KOH (694 mg, 12.3 mmol, 2.00 eq) at 25 °C was degassed and purged with CO (6.19 mmol, 1.00 eq) for 5 times. The mixture was stirred at 110 °C for 12 h under CO (6.19 mmol, 1.00 eq). One batch in parallel was monitored in random. LC-MS showed desired mass was detected. Two batches in parallel were combined. The reaction mixture was diluted with H₂O 50 mL and extracted with EtOAc (50 mL*3). The combined organic layers were filtered and concentrated under reduced pressure. The residue was purified by column chromatography (SiO₂, Petroleum ether:Ethyl acetate=1:0 to 1:1) to give the title compound (2.60 g, 7.11 mmol, 57.5% yield, 87.3% purity) as a white solid. LCMS (ES⁺): m/z 319.9 [M+H]⁺.

Synthesis of tert-butyl 4-(3-(2,6-dioxopiperidin-3-yl)-2-oxo-2,3-dihydrobenzo[d]oxazol-6-yl)piperazine-1-carboxylate. To a solution of tert-butyl 4-(2-oxo-2,3-dihydrobenzo[d]oxazol-6-yl)piperazine-1-carboxylate (2 g, 6.26 mmol, 1.00 eq) in DMF (20 mL) was added Cs₂CO₃ (6.12 g, 18.7 mmol, 3.00 eq) and 3-bromopiperidine-2,6-dione (3.61 g, 18.7 mmol, 3.00 eq) at 25 °C. The mixture was stirred at 80 °C for 12 h. LC-MS showed desired mass was detected. The reaction mixture was diluted with H₂O 50 mL and extracted with EtOAc (50 mL*3). The combined organic layers were filtered and concentrated under reduced pressure. The residue was purified by column chromatography (SiO₂, Petroleum ether:Ethyl acetate=1:0 to 1:1) to give the title compound (1.50 g, 3.45 mmol, 55.0% yield, 99.0% purity) as a gray solid. LCMS (ES⁺): m/z 431.1 [M+H]⁺.

Synthesis of 3-(2-oxo-6-(piperazin-1-yl)benzo[d]oxazol-3 (2H)-yl)piperidine-2,6-dione. To a solution of tert-butyl 4-(3-(2,6-dioxopiperidin-3-yl)-2-oxo-2,3-dihydrobenzo[d]oxazol-6-yl)piperazine-1-carboxylate (1.40 g, 3.25 mmol, 1.00 eq) in DCM (15 mL) was added TFA (7.68 g, 67.3 mmol, 5 mL, 20.7 eq) at 25 °C. The mixture was stirred at 25 °C for 2 h. LC-MS showed desired mass was detected. The reaction concentrated under reduced pressure to give the title compound (2.30 g, crude, TFA) was obtained as a purple oil. LCMS (ES⁺): m/z, 331.2 [M+H]⁺.

Synthesis of tert-butyl 4-(2-(4-(3-(2,6-dioxopiperidin-3-yl)-2-oxo-2,3-dihydrobenzo[d]oxazol-6-yl)piperazin-1-yl)ethyl)piperidine-1-carboxylate. To a solution of 3-(2-oxo-6-(piperazin-1-yl)benzo[d]oxazol-3 (2H)-yl)piperidine-2,6-dione (700 mg, 1.58 mmol, 1.00 eq, TFA) in DCE (20 mL) was added DIEA (407 mg, 3.15 mmol, 548 μL, 2.00 eq) at 25 °C. The mixture was stirred at 25 °C for 0.5 h. Then Ti(i-PrO)₄ (2.24 g, 7.88 mmol, 2.32 mL, 5.00 eq) and tert-butyl 4-(2-oxoethyl)piperidine-1-carboxylate (1.07 g, 4.73 mmol, 3.00 eq) were added at 25 °C. The mixture was stirred at 25 °C for 0.5 h. Then NaBH₃CN (494 mg, 7.88 mmol, 5.00 eq) was added at 25 °C. The mixture was stirred at 40 °C for 12 h. LC-MS showed desired mass was detected. The reaction mixture was diluted with H₂O 50 mL and filtered. The filter liquor was extracted with EtOAc (50 mL*3). The combined organic layers were concentrated under reduced pressure. The residue was purified by prep-TLC (SiO₂, Petroleum ether:THF=0:1) to give the residue. The residue was purified by prep-HPLC (column: Phenomenex luna C18 100*40 mm*5 um; mobile phase: [H₂O (0.2% FA)-ACN]; gradient: 5%-50% B over 8.0 min) to give the title compound (60.0 mg, 92.8 μmol, 10.0% yield, 83.7% purity) was obtained as a white solid. LCMS (ES⁺): m/z 542.3 [M+H]⁺.

Synthesis of 3-(2-oxo-6-(4-(2-(piperidin-4-yl)ethyl)piperazin-1-yl)benzo[d]oxazol-3 (2H)-yl)piperidine-2,6-dione. To a solution of tert-butyl 4-(2-(4-(3-(2,6-dioxopiperidin-3-yl)-2-oxo-2,3-dihydrobenzo[d]oxazol-6-yl)piperazin-1-yl)ethyl)piperidine-1-carboxylate (60.0 mg, 110 μmol, 1.00 eq) in DCM (3 mL) was added TFA (1.54 g, 13.4 mmol, 1 mL, 121 eq) at 25 °C. The mixture was stirred at 25 °C for 1 h. LC-MS showed desired mass was detected. The reaction concentrated under reduced pressure to give the title compound (60.0 mg, crude, TFA) as a white oil. LCMS (ES⁺): m/z 442.2 [M+H]⁺.

Synthesis of cis-3-(6-(4-(2-(1-(3-((5-fluoro-4-(3-(2-oxopyridin-1(2H)-yl)phenyl)pyrimidin-2-yl)amino)cyclohexane-1-carbonyl)piperidin-4-yl)ethyl)piperazin-1-yl)-2-oxobenzo[d]oxazol-3 (2H)-yl)piperidine-2,6-dione (Compound 342). To a solution of 3-(2-oxo-6-(4-(2-(piperidin-4-yl)ethyl)piperazin-1-yl)benzo[d]oxazol-3 (2H)-yl)piperidine-2,6-dione (40.0 mg, 72.0 μmol, 1.00 eq, TFA) in DMF (2 mL) was added cis-3-((5-fluoro-4-(3-(2-oxopyridin-1(2H)-yl)phenyl)pyrimidin-2-yl)amino)cyclohexane-1-carboxylic acid (44.1 mg, 108 μmol, 1.50 eq) DIEA (27.9 mg, 216 μmol, 37.6 μL, 3.00 eq) and T₄P (77.8 mg, 108 μmol, 50.0% purity, 1.50 eq) at 25 °C. The mixture was stirred at 25 °C for 1 h. LC-MS showed desired mass was detected. The reaction mixture was concentrated under reduced pressure. The residue was purified by prep-HPLC (column: Phenomenex Luna C18 75*30 mm*3 um; mobile phase: [H₂O (0.1% TFA)-ACN]; gradient: 15%-40% B over 8.0 min) to give the title compound (Compound 342) as a yellow solid. LCMS (ES⁺): m/z 832.3 [M+H]⁺.

### Example 335. Synthesis of 3-(6-(4-(2-(4-(cis-3-((5-fluoro-4-(3-(2-oxopyridin-1(2H)-yl)phenyl)pyrimidin-2-yl)amino)cyclohexane-1-carbonyl)piperazin-1-yl)ethyl)piperidin-1-yl)-2-oxobenzo[d]oxazol-3 (2H)-yl)piperidine-2,6-dione (Compound 386)

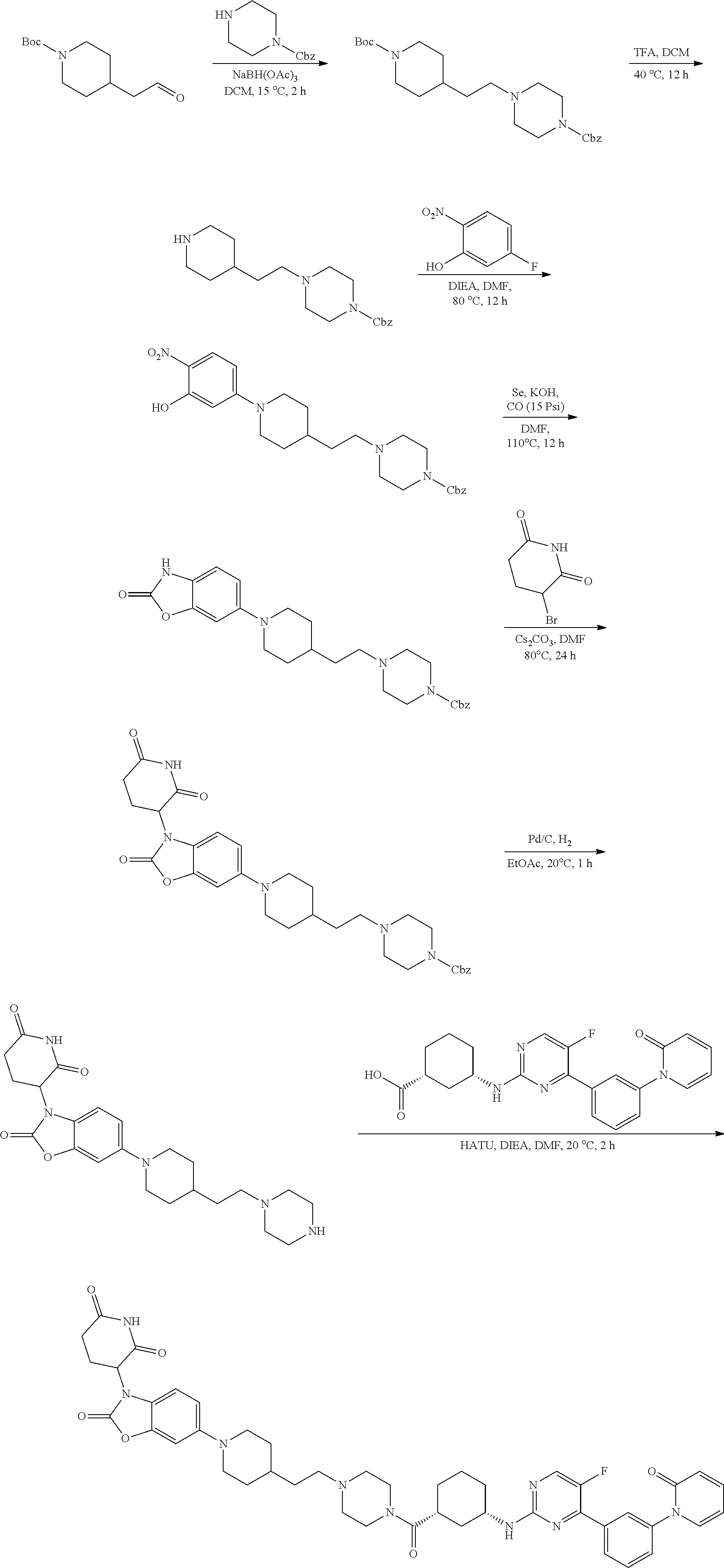

Synthesis of benzyl 4-(2-(1-(tert-butoxycarbonyl)piperidin-4-yl)ethyl)piperazine-1-carboxylate. To a solution of tert-butyl 4-(2-oxoethyl)piperidine-1-carboxylate (3.60 g, 15.8 mmol, 1.00 eq) and benzyl piperazine-1-carboxylate (3.49 g, 15.8 mmol, 3.05 mL, 1.00 eq) in DCM (50 mL) was added NaBH(OAc)₃ (10.1 g, 47.5 mmol, 3.00 eq) at 15 °C. The mixture was stirred at 15 °C for 2 h. LC-MS showed that tert-butyl 4-(2-oxoethyl)piperidine-1-carboxylate was consumed completely and one main peak with desired mass was detected. The mixture was diluted with H₂O (150 mL) and extracted with DCM (50 mL*3). The organic layers were washed with brine (150 mL), dried over Na₂SO₄, filtered and concentrated under reduced pressure. The residue was purified by prep-TLC (SiO₂, EtOAc:MeOH=10:1) to give the title compound (7.00 g, crude) as a yellow oil. LCMS (ES⁺): m/z 432.3 [M+H]⁺.

Synthesis of benzyl 4-(2-(piperidin-4-yl)ethyl)piperazine-1-carboxylate. A solution of benzyl benzyl 4-(2-(1-(tert-butoxycarbonyl)piperidin-4-yl)ethyl)piperazine-1-carboxylate (7.00 g, 16.2 mmol, 1.00 eq) in DCM (15 mL) and TFA (7.68 g, 67.3 mmol, 5 mL, 4.15 eq) was stirred at 40 °C for 12 h. LC-MS showed that benzyl benzyl 4-(2-(1-(tert-butoxycarbonyl)piperidin-4-yl)ethyl)piperazine-1-carboxylate was consumed completely and one main peak with desired mass was detected. The mixture was concentrated under reduced pressure to give the title compound (7.50 g, crude, TFA) as a brown oil. LCMS (ES⁺): m/z 332.3 [M+H]⁺.

Synthesis of benzyl 4-(2-(1-(3-hydroxy-4-nitrophenyl)piperidin-4-yl)ethyl)piperazine-1-carboxylate. To a solution of benzyl 4-(2-(piperidin-4-yl)ethyl)piperazine-1-carboxylate (7.50 g, 16.8 mmol, 1.00 eq, TFA) and 5-fluoro-2-nitrophenol (2.64 g, 16.8 mmol, 1.00 eq) in DMF (40 mL) was added DIEA (6.53 g, 50.5 mmol, 8.80 mL, 3.00 eq) at 15 °C. The mixture was stirred at 80 °C for 12 h. LC-MS showed that benzyl 4-(2-(piperidin-4-yl)ethyl)piperazine-1-carboxylate was consumed completely and one main peak with desired mass was detected. The mixture was diluted with H₂O (100 mL) and extracted with EtOAc (30 mL*3). The organic layers were washed with brine (100 mL), dried over Na₂SO₄, filtered and concentrated under reduced pressure. The residue was purified by MPLC (SiO₂, Petroleum ether/Ethyl acetate=1/0 to 0/1) to give title compound (2.60 g, 5.55 mmol, 33.0% yield, n/a purity) as a yellow solid. LCMS (ES⁺): m/z 469.2 [M+H]⁺.

Synthesis of benzyl 4-(2-(1-(2-oxo-2,3-dihydrobenzo[d]oxazol-6-yl)piperidin-4-yl)ethyl)piperazine-1-carboxylate. The mixture was set up in five batches. To a solution of benzyl 4-(2-(1-(3-hydroxy-4-nitrophenyl)piperidin-4-yl)ethyl)piperazine-1-carboxylate (500 mg, 1.07 mmol, 1.00 eq) in DMF (5 mL) was added Se (86.5 mg, 1.07 mmol, 1.00 eq) and KOH (120 mg, 2.14 mmol, 2.00 eq) at 15 °C. The mixture was stirred at 110 °C for 2 h under CO (15 psi). LC-MS showed that benzyl 4-(2-(1-(3-hydroxy-4-nitrophenyl)piperidin-4-yl)ethyl)piperazine-1-carboxylate consumed completely and one main peak with desired mass was detected. Five batches were combined to work up. The mixture was filtered and the filtrate was concentrate under reduced pressure. The residue was purified by reverse-MPLC (FA condition) to give title compound (1.00 g, 2.15 mmol, 40.3% yield, N/A purity) as a yellow solid. LCMS (ES⁴): m/z 465.2 [M+H]⁺.

Synthesis of rac-benzyl (R)-4-(2-(1-(3-(2,6-dioxopiperidin-3-yl)-2-oxo-2,3-dihydrobenzo[d]oxazol-6-yl)piperidin-4-yl)ethyl)piperazine-1-carboxylate. To a solution of benzyl 4-(2-(1-(2-oxo-2,3-dihydrobenzo[d]oxazol-6-yl)piperidin-4-yl)ethyl)piperazine-1-carboxylate (100 mg, 215 μmol, 1.00 eq) and rac-3-bromopiperidine-2,6-dione (124 mg, 646 μmol, 3.00 eq) in DMF (2 mL) was added Cs₂CO₃ (140 mg, 431 μmol, 2.00 eq) at 15 °C. The mixture was stirred at 80 °C for 12 h. LC-MS showed that benzyl 4-(2-(1-(2-oxo-2,3-dihydrobenzo[d]oxazol-6-yl)piperidin-4-yl)ethyl)piperazine-1-carboxylate remained and one peak with desired mass was detected. 3-bromopiperidine-2,6-dione (82.7 mg, 431 μmol, 2.00 eq) and Cs₂CO₃ (70.1 mg, 215 μmol, 1.00 eq) was added to the mixture at 15 °C. The mixture was stirred at 80 °C for 12 h. LC-MS showed that benzyl 4-(2-(1-(2-oxo-2,3-dihydrobenzo[d]oxazol-6-yl)piperidin-4-yl)ethyl)piperazine-1-carboxylate remained and one peak with desired mass was detected. The mixture was filtered and the filtrate was concentrated under reduced pressure. The residue was purified by prep-HPLC (column: Phenomenex luna C18 100*40 mm*5 um; mobile phase: [H₂O (0.2% FA)-ACN]; gradient: 1%-30% B over 8.0 min) to give the title compound (50.0 mg, 86.9 μmol, 40.4% yield, n/a purity) as a blue solid. LCMS (ES⁺): m/z 576.3 [M+H]⁺.

Synthesis of rac-3-(2-oxo-6-(4-(2-(piperazin-1-yl)ethyl)piperidin-1-yl)benzo[d]oxazol-3 (2H)-yl)piperidine-2,6-dione. To a mixture of 10% Pd/C (50.0 mg, 235 mol, 50% purity, 2.70 eq) in 2,2,2-trifluoroethanol (6 mL) was added rac-benzyl (R)-4-(2-(1-(3-(2,6-dioxopiperidin-3-yl)-2-oxo-2,3-dihydrobenzo[d]oxazol-6-yl)piperidin-4-yl)ethyl)piperazine-1-carboxylate (50.0 mg, 86.9 μmol, 1.00 eq) at 20 °C. The mixture was stirred at 20 °C for 1 h under H₂ (15 Psi). LC-MS showed that rac-benzyl (R)-4-(2-(1-(3-(2,6-dioxopiperidin-3-yl)-2-oxo-2,3-dihydrobenzo[d]oxazol-6-yl)piperidin-4-yl)ethyl)piperazine-1-carboxylate was consumed completely and one main peak with the desired mass was detected. The mixture was filtered and the filtrate was concentrated under reduced pressure to give the title compound (60.0 mg, crude) was obtained as a purple solid. LCMS (ES⁺): m/z 442.2 [M+H]⁺.

Synthesis of 3-(6-(4-(2-(4-(cis-3-((5-fluoro-4-(3-(2-oxopyridin-1(2H)-yl)phenyl)pyrimidin-2-yl)amino)cyclohexane-1-carbonyl)piperazin-1-yl)ethyl)piperidin-1-yl)-2-oxobenzo[d]oxazol-3 (2H)-yl)piperidine-2,6-dione (Compound 386). To a solution of rac-3-(2-oxo-6-(4-(2-(piperazin-1-yl)ethyl)piperidin-1-yl)benzo[d]oxazol-3 (2H)-yl)piperidine-2,6-dione (40.0 mg, 90.6 μmol, 1.00 eq) in DMF (2 mL) was added cis-3-((5-fluoro-4-(3-(2-oxopyridin-1(2H)-yl)phenyl)pyrimidin-2-yl)amino)cyclohexane-1-carboxylic acid (37.0 mg, 90.6 μmol, 1.00 eq) and DIEA (58.5 mg, 453 μmol, 78.9 μL, 5.00 eq), HATU (103 mg, 271 mol, 3.00 eq) at 20 °C. The mixture was stirred at 20 °C for 2 h. LC-MS showed that rac-3-(2-oxo-6-(4-(2-(piperazin-1-yl)ethyl)piperidin-1-yl)benzo[d]oxazol-3 (2H)-yl)piperidine-2,6-dione was consumed completely and one peak with the desired mass was detected. The reaction mixture was purified by prep-HPLC (column: Phenomenex Luna C18 100*30 mm*3 um; mobile phase: [H₂O (0.2% FA)-ACN]; gradient: 1%-30% B over 8.0 min) to give the title compound (Compound 386) (6.60 mg, 7.22 μmol, 7.97% yield, 91.0% purity) as a yellow solid. LCMS (ES⁺): m/z 832.3 [M+H]⁺.

### Example 336. Synthesis of 3-((3-fluoro-4-(1-(2-(1-((1R,3S)-3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)phenyl)amino)piperidine-2,6-dione (Compound 308)

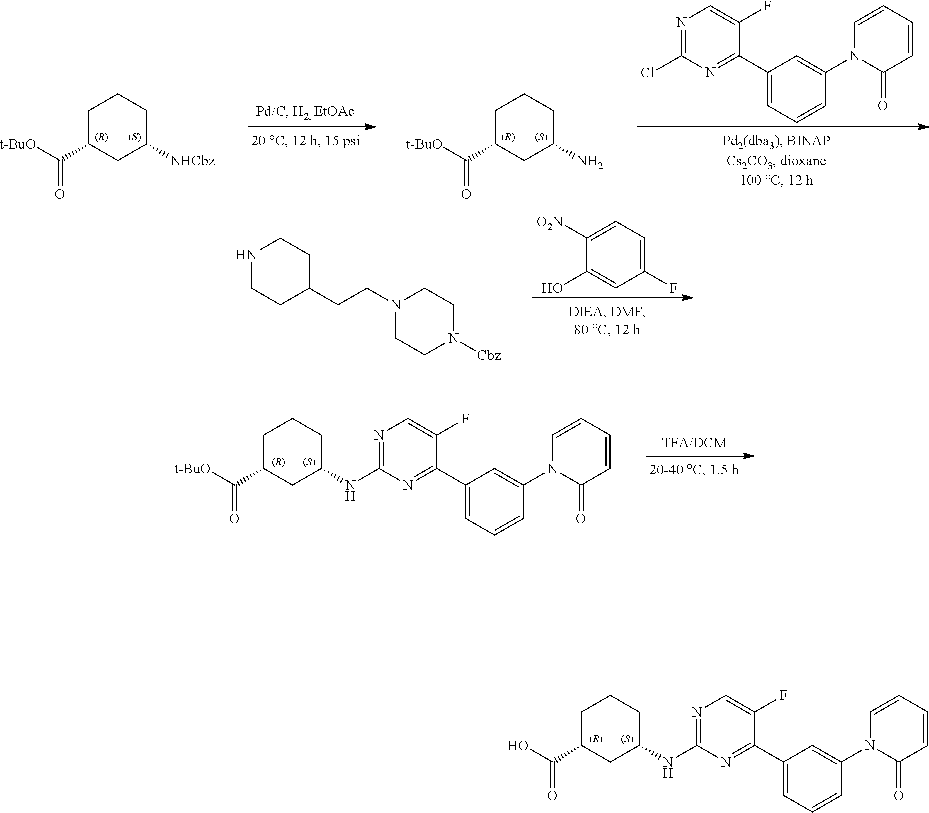

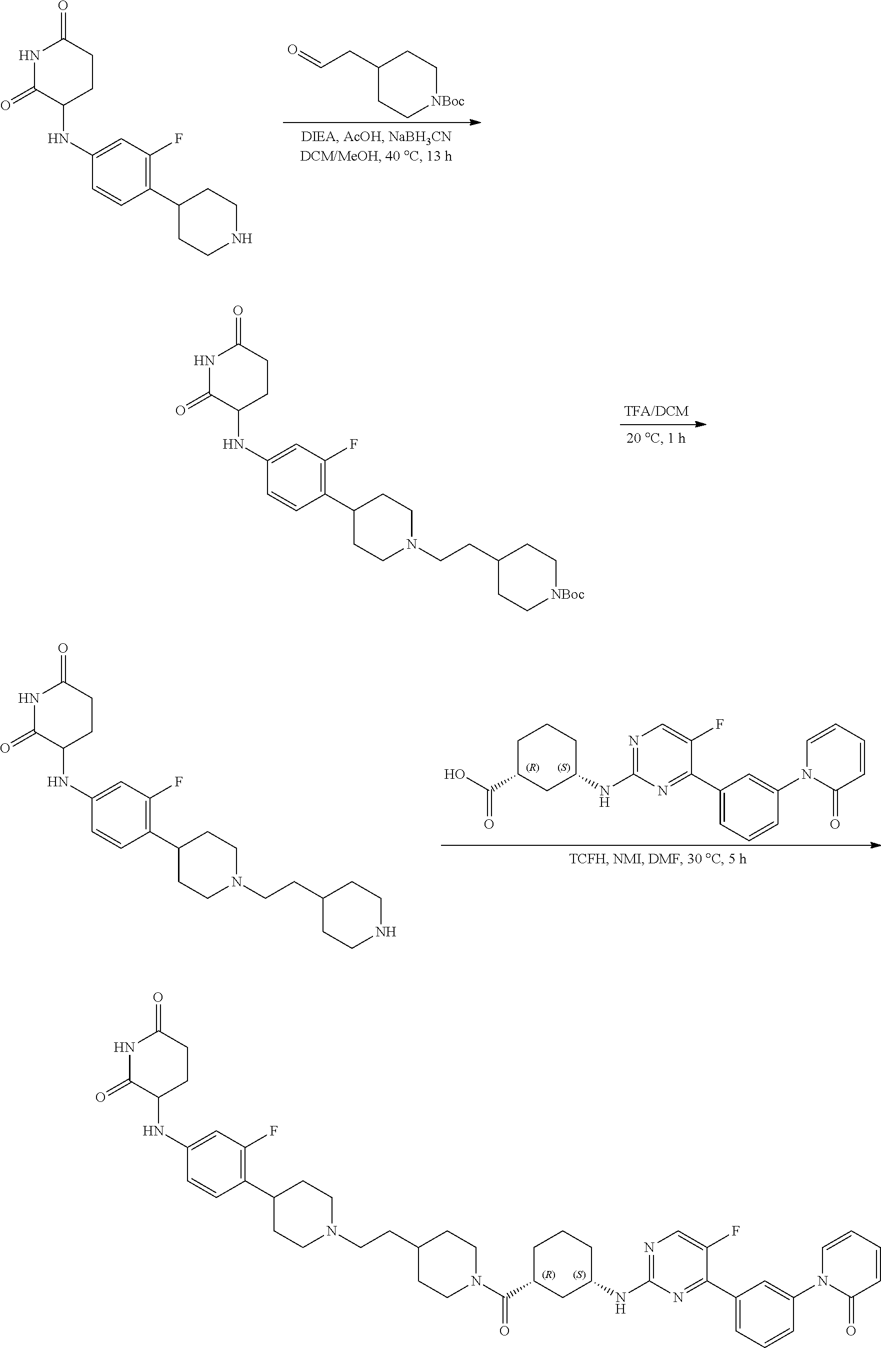

Synthesis of tert-butyl (1R,3S)-3-aminocyclohexane-1-carboxylate. To a solution of tert-butyl (1R,3S)-3-(((benzyloxy)carbonyl)amino)cyclohexane-1-carboxylate (2.00 g, 6.00 mmol, 1.00 eq) in EtOAc (10 mL) was added 10% Pd/C (4.00 g, 50% purity) at 20 °C under N₂. Then the mixture was degassed and purged with H₂, then it was stirred at 20 °C for 12 h under H₂ (15 psi) atmosphere. LCMS showed tert-butyl (1R,3S)-3-(((benzyloxy) carbonyl)amino)cyclohexane-1-carboxylate was consumed and the desired mass was detected. The mixture was filtered and the filtrate was concentrated under reduced pressure to give the title compound (1 g, crude) as a white solid. LCMS (ES⁺): m/z 200.2 [M+H]⁺

Synthesis of tert-butyl (1R,3S)-3-((5-fluoro-4-(3-(2-oxopyridin-1(2H)-yl)phenyl)pyrimidin-2-yl)amino)cyclohexane-1-carboxylate. To a solution of 1-(3-(2-chloro-5-fluoropyrimidin-4-yl)phenyl)pyridin-2(1H)-one (400 mg, 1.33 mmol, 1.00 eq) and tert-butyl (1R,3S)-3-aminocyclohexane-1-carboxylate (476 mg, 2.39 mmol, 1.80 eq) in dioxane (10 mL) were added Cs₂CO₃ (1.30 g, 3.98 mmol, 3.00 eq), BINAP (74.30 mg, 119.32 μmol, 0.0900 eq) and Pd₂(dba)₃ (60.7 mg, 66.3 μmol, 0.0500 eq) at 20 °C under N₂ atmosphere. The mixture was stirred at 100 °C for 12 h under N₂ atmosphere. LCMS showed tert-butyl (1R,3S)-3-aminocyclohexane-1-carboxylate was consumed completely and the desired target MS was detected. H₂O (10 mL) was added to the mixture and the mixture was extracted with EtOAc (10 mL*3), the combined organic layers were washed with brine (10 mL*3), dried over Na₂SO₄, filtered and the filtrate was concentrated under reduced pressure to give a residue. The residue was purified by prep-TLC (SiO₂, PE/EtOAc=0/1) to give the title compound (350 mg, 753 μmol, 56.8% yield) as a yellow oil. LCMS (ES⁺): m/z 465.3 [M+H]⁺

Synthesis of (1R,3S)-3-((5-fluoro-4-(3-(2-oxopyridin-1(2H)-yl)phenyl)pyrimidin-2-yl)amino)cyclohexane-1-carboxylic acid. A solution of tert-butyl (1R,3S)-3-((5-fluoro-4-(3-(2-oxopyridin-1(2H)-yl)phenyl)pyrimidin-2-yl)amino)cyclohexane-1-carboxylate (350 mg, 753 μmol, 1.00 eq) in DCM (4 mL) and TFA (0.8 mL) was stirred at 20 °C for 1 h. LCMS showed tert-butyl (1R,3S)-3-((5-fluoro-4-(3-(2-oxopyridin-1(2H)-yl)phenyl)pyrimidin-2-yl)amino)cyclohexane-1-carboxylate remained and the desired target MS was detected. Then the mixture was stirred at 40 °C for another 0.5 h. LCMS showed tert-butyl (1R,3S)-3-((5-fluoro-4-(3-(2-oxopyridin-1(2H)-yl)phenyl)pyrimidin-2-yl)amino)cyclohexane-1-carboxylate was consumed completely and the desired target MS was detected. The mixture was concentrated under reduced pressure to give the title compound (300 mg, crude) as a yellow oil. LCMS (ES⁺): m/z 409.2 [M+H]⁺

Synthesis of tert-butyl 4-(2-(4-(4-((2,6-dioxopiperidin-3-yl)amino)-2-fluorophenyl)piperidin-1-yl)ethyl)piperidine-1-carboxylate. To a solution of 3-((3-fluoro-4-(piperidin-4-yl)phenyl)amino)piperidine-2,6-dione (1.20 g, 2.86 mmol, 1.00 eq, TFA) in DCM (12 mL) and MeOH (2 mL) was added DIEA to adjust pH to 8 at 20 °C. The mixture was stirred at 20 °C for 0.5 h. AcOH (172 mg, 2.86 mmol, 164 μL, 1.00 eq) and tert-butyl 4-(2-oxoethyl)piperidine-1-carboxylate (1.95 g, 8.58 mmol, 3.00 eq) were added to the mixture at 20 °C. The mixture was stirred at 20 °C for 0.5 h, pH was 5 now. Then NaBH₃CN (539 mg, 8.58 mmol, 3.00 eq) was added to the mixture at 20 °C. The mixture was stirred at 40 °C for 12 h. LCMS showed 3-((3-fluoro-4-(piperidin-4-yl)phenyl)amino)piperidine-2,6-dione was consumed completely and the desired mass was detected. The mixture was diluted with H₂O (30 mL) and then extracted with EtOAc (20 mL*3). The combined organic layers were dried over Na₂SO₄, filtered and the filtrate was concentrated under reduced pressure to give a residue. The residue was purified by column chromatography (SiO₂, dichloromethane/Ethyl acetate=1/0 to 1/3) to give the title compound (1.06 g, 2.05 mmol, 71.7% yield) as a yellow oil. LCMS (ES⁺): m/z 517.4 [M+H]⁺

Synthesis of 3-((3-fluoro-4-(1-(2-(piperidin-4-yl)ethyl)piperidin-4-yl)phenyl)amino)piperidine-2,6-dione. A solution of tert-butyl 4-(2-(4-(4-((2,6-dioxopiperidin-3-yl)amino)-2-fluorophenyl)piperidin-1-yl)ethyl)piperidine-1-carboxylate (300 mg, 581 μmol, 1.00 eq) in DCM (3 mL) and TFA (0.6 mL) was stirred at 20 °C for 1 h. LCMS showed tert-butyl 4-(2-(4-(4-((2,6-dioxopiperidin-3-yl)amino)-2-fluorophenyl)piperidin-1-yl)ethyl)piperidine-1-carboxylate was consumed and the desired target MS was detected. The mixture was concentrated under reduced pressure to give the title compound (300 mg, crude, TFA) as a yellow oil. LCMS (ES⁺): m/z 417.3 [M+H]⁺

Synthesis of 3-((3-fluoro-4-(1-(2-(1-((1R,3S)-3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)phenyl)amino)piperidine-2,6-dione (Compound 308). To a solution of (1R,3S)-3-((5-fluoro-4-(3-(2-oxopyridin-1(2H)-yl)phenyl)pyrimidin-2-yl)amino)cyclohexane-1-carboxylic acid (129 mg, 317 μmol, 0.600 eq) in DMF (3 mL) were added NMI (130 mg, 1.58 mmol, 126 μL, 3.00 eq) and TCFH (178 mg, 633 μmol, 1.20 eq) at 20 °C, then 3-((3-fluoro-4-(1-(2-(piperidin-4-yl)ethyl)piperidin-4-yl)phenyl)amino)piperidine-2,6-dione (280 mg, 528 μmol, 1.00 eq, TFA) was added to the mixture, the mixture was stirred at 30 °C for 2 h. LCMS showed 3-((3-fluoro-4-(1-(2-(piperidin-4-yl)ethyl)piperidin-4-yl)phenyl)amino)piperidine-2,6-dione remained and the desired target MS was detected. Then TCFH (178 mg, 633 μmol, 1.20 eq) and NMI (130 mg, 1.58 mmol, 126 μL, 3.00 eq) were added to the mixture, the mixture was stirred at 30 °C for another 3 h. LCMS showed 3-((3-fluoro-4-(1-(2-(piperidin-4-yl)ethyl)piperidin-4-yl)phenyl)amino)piperidine-2,6-dione was consumed completely and the desired target MS was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to give the title compound (Compound 308) (5.20 mg, 6.44 μmol, 1.22% yield) as a white solid. LCMS (ES⁺): m/z 807.4 [M+H]⁺

### Example 337. Synthesis of 3-[4-[3,3-difluoro-1-[2-[1-[(1R,3S)-3-[[5-fluoro-4-[3-(2-oxo-1-pyridyl)phenyl]pyrimidin-2-yl]amino]cyclohexanecarbonyl]-4-piperidyl]ethyl]-4-piperidyl]-3-fluoro-anilino]piperidine-2,6-dione (Compound 310)

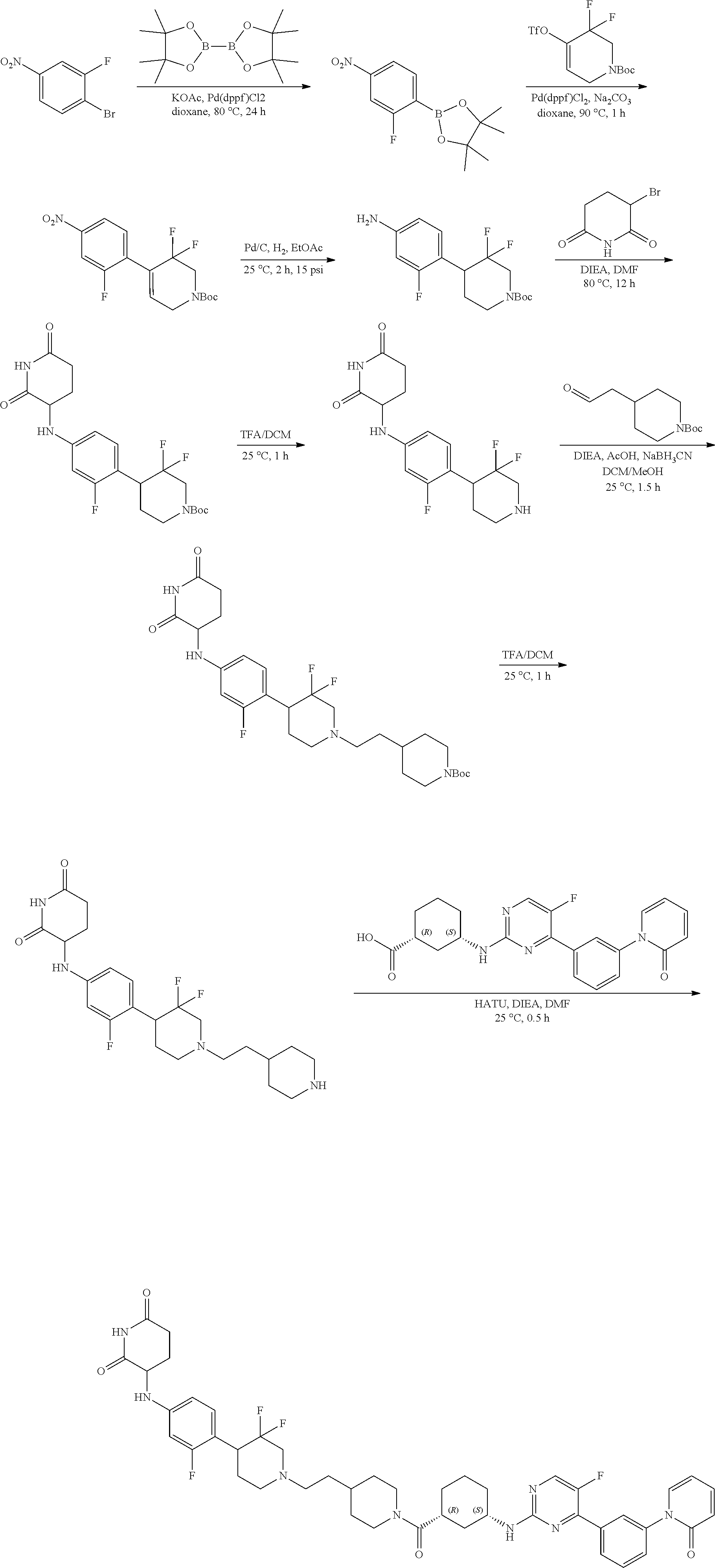

Synthesis of 2-(2-fluoro-4-nitrophenyl)-4,4,5,5-tetramethyl-1,3,2-dioxaborolane. To a solution of 1-bromo-2-fluoro-4-nitrobenzene (35.0 g, 159 mmol, 1.00 eq) in dioxane (2000 mL) were added KOAc (31.2 g, 318 mmol, 2.00 eq), 4,4,4′,4′,5,5,5′,5′-octamethyl-2,2′-bi(1,3,2-dioxaborolane) (48.5 g, 191 mmol, 1.20 eq) and Pd(dppf)Cl₂ (5.82 g, 7.95 mmol, 0.0500 eq) at 20 °C under N₂ atmosphere. The mixture was stirred at 80 °C for 24 h under N₂ atmosphere. TLC (SiO₂, PE/EtOAc=5/1) showed 1-bromo-2-fluoro-4-nitrobenzene was consumed completely and two new spots formed. TLC (SiO₂, PE/EtOAc=5/1) indicated 1-bromo-2-fluoro-4-nitrobenzene was consumed completely and two new spots formed. The reaction mixture was concentrated under reduced pressure to give the title compound (130 g, crude) as a black oil. The product couldn't be detected by LCMS. It was confirmed by ¹H NMR (400 MHz, DMSO-d₆) δ=8.08 (d, J=8.3 Hz, 1H), 8.02 (d, J=9.0 Hz, 1H), 7.94-7.89 (m, 1H), 1.33 (s, 12H)

Synthesis of tert-butyl 3,3-difluoro-4-(2-fluoro-4-nitro-phenyl)-2,6-dihydropyridine-1-carboxylate. To a solution of 2-(2-fluoro-4-nitro-phenyl)-4,4,5,5-tetramethyl-1,3,2-dioxaborolane (5.00 g, 18.7 mmol, 1.20 eq) and tert-butyl 3,3-difluoro-4-(trifluoromethylsulfonyloxy)-2,6-dihydropyridine-1-carboxylate (5.73 g, 15.6 mmol, 1.00 eq) in dioxane (80 mL) were added Na₂CO₃ (3.31 g, 31.2 mmol, 2.00 eq) and Pd(dppf)Cl₂ (1.14 g, 1.56 mmol, 0.100 eq) at 25 °C, the mixture was stirred at 90 °C for 1 h. LCMS showed the desired mass was detected. To the mixture was added H₂O (50 mL) and the mixture was extracted with EtOAc (50 mL*3). The combined organic layers were washed with brine (50 mL*3), dried over Na₂SO₄, filtered and the filtrate was concentrated under reduced pressure to give a residue. The residue was purified by MPLC (SiO₂, Petroleum ether/Ethyl acetate=3/1) to give the title compound (2.80 g, 7.81 mmol, 50.1% yield) as a yellow solid. LCMS (ES⁺): m/z 344.0 [M+H−56+MeCN]⁺.

Synthesis of tert-butyl 4-(4-amino-2-fluoro-phenyl)-3,3-difluoro-piperidine-1-carboxylate. To a solution of tert-butyl 3,3-difluoro-4-(2-fluoro-4-nitro-phenyl)-2,6-dihydropyridine-1-carboxylate (1.35 g, 3.77 mmol, 1.00 eq) in EtOAc (20 mL) was added 10% Pd/C (1.50 g, 50% purity) at 25 °C, the mixture was degassed and purged with H₂ for three times. Then it was stirred at 25 °C for 2 h under H₂ (15 psi). LCMS showed that the desired mass was detected. The mixture was filtered and the filtrate was concentrated under reduced pressure to give the title compound (1.23 g, crude) as a yellow solid. LCMS (ES⁺): m/z 275.0 [M+H−56]⁺.

Synthesis of tert-butyl 4-[4-[(2,6-dioxo-3-piperidyl)amino]-2-fluoro-phenyl]-3,3-difluoro-piperidine-1-carboxylate. To a solution of tert-butyl 4-(4-amino-2-fluoro-phenyl)-3,3-difluoro-piperidine-1-carboxylate (1.20 g, 3.63 mmol, 1.00 eq) and 3-bromopiperidine-2,6-dione (2.09 g, 10.9 mmol, 3.00 eq) in DMF (20 mL) was added DIEA (1.41 g, 10.9 mmol, 1.90 mL, 3.00 eq) at 25 °C, the mixture was stirred at 80 °C for 12 h. LCMS showed the desired mass was detected. To the mixture was added H₂O (30 mL) and the mixture was extracted with EtOAc (30 mL*3). The combined organic layers were washed with brine (30 mL*3), dried over Na₂SO₄, filtered and the filtrate was concentrated under reduced pressure to give a residue. The residue was purified by prep-TLC (SiO₂, PE/EtOAc=1/1) to give the title compound (180 mg, 407 μmol, 11.2% yield) as a green solid. LCMS (ES⁺): m/z 386.1 [M+H−56]⁺.

Synthesis of 3-[4-(3,3-difluoro-4-piperidyl)-3-fluoro-anilino]piperidine-2,6-dione. To a solution of tert-butyl 4-[4-[(2,6-dioxo-3-piperidyl)amino]-2-fluoro-phenyl]-3,3-difluoro-piperidine-1-carboxylate (180 mg, 408 μmol, 1.00 eq) in DCM (4 mL) was added TFA (1 mL) at 25 °C, the mixture was stirred at 25 °C for 1 h. LCMS showed that the desired mass was detected. The mixture was concentrated under reduced pressure to give the title compound (190 mg, crude, TFA) as a green oil liquid. LCMS (ES⁺): m/z 342.1 [M+H]⁺.

Synthesis of tert-butyl 4-[2-[4-[4-[(2,6-dioxo-3-piperidyl)amino]-2-fluoro-phenyl]-3,3-difluoro-1-piperidyl]ethyl]piperidine-1-carboxylate. To a solution of tert-butyl 4-(2-oxoethyl)piperidine-1-carboxylate (90.0 mg, 395 μmol, 1.00 eq) in DCM (5 mL) and MeOH (5 mL) was added DIEA to adjust pH to 8 at 25 °C, the mixture was stirred at 25 °C for 0.5 h, then AcOH and 3-[4-(3,3-difluoro-4-piperidyl)-3-fluoro-anilino]piperidine-2,6-dione (180 mg, 395 μmol, 1.00 eq, TFA) was added at 25 °C, the pH was 5 now, the mixture was stirred at 40 °C for 0.5 h. Then NaBH₃CN (37.3 mg, 593 μmol, 1.50 eq) was added at 40 °C, the mixture was stirred at 40 °C for 0.5 h. LCMS showed the desired mass was detected. To the mixture was added H₂O (5 mL) and the mixture was extracted with EtOAc (10 mL*3). The combined organic layers were washed with brine (10 mL*3), dried over Na₂SO₄, filtered and the filtrate was concentrated under reduced pressure to give the title compound (230 mg, crude) as a yellow solid. LCMS (ES⁺): m/z 553.3 [M+H]⁺

Synthesis of 3-[4-[3,3-difluoro-1-[2-(4-piperidyl)ethyl]-4-piperidyl]-3-fluoro-anilino]piperidine-2,6-dione. To a solution of tert-butyl 4-[2-[4-[4-[(2,6-dioxo-3-piperidyl)amino]-2-fluoro-phenyl]-3,3-difluoro-1-piperidyl]ethyl]piperidine-1-carboxylate (100 mg, 181 μmol, 1.00 eq) in DCM (4 mL) was added TFA (1 mL) at 25 °C, the mixture was stirred at 25 °C for 1 h. LCMS showed the desired mass was detected. The mixture was concentrated under reduced pressure to give the title compound (100 mg, crude, TFA) as a yellow oil liquid. LCMS (ES⁺): m/z 453.2 [M+H]⁺.

Synthesis of 3-[4-[3,3-difluoro-1-[2-[1-[(1R,3S)-3-[[5-fluoro-4-[3-(2-oxo-1-pyridyl)phenyl]pyrimidin-2-yl]amino]cyclohexanecarbonyl]-4-piperidyl]ethyl]-4-piperidyl]-3-fluoro-anilino]piperidine-2,6-dione (Compound 310). To a solution of (1R,3S)-3-[[5-fluoro-4-[3-(2-oxo-1-pyridyl)phenyl]pyrimidin-2-yl]amino]cyclohexanecarboxylic acid (72.1 mg, 177 μmol, 1.00 eq) in DMF (3 mL) were added DIEA (45.6 mg, 353 μmol, 61.5 μL, 2.00 eq) and HATU (100 mg, 265 μmol, 1.50 eq) at 25 °C, the mixture was stirred at 25 °C for 15 min. 3-[4-[3,3-difluoro-1-[2-(4-piperidyl)ethyl]-4-piperidyl]-3-fluoro-anilino]piperidine-2,6-dione (100 mg, 177 μmol, 1.00 eq, TFA) was added at 25 °C, the mixture was stirred at 25 °C for 15 min. LCMS showed the desired mass was detected. To the mixture was added H₂O (5 mL) and the mixture was extracted with EtOAc (5 mL*3). The combined organic layers were washed with brine (10 mL*3), dried over Na₂SO₄, filtered and the filtrate was concentrated under reduced pressure to give a residue. The residue was purified by prep-HPLC (column: Phenomenex luna C18 100*40 mm*3 um; mobile phase: [H₂O (0.2% FA)-ACN]; gradient: 10%-50% B over 8.0 min) to give the title compound (Compound 310) (32.4 mg, 37.9 μmol, 21.5% yield, 98.7% purity) as a white solid. LCMS (ES⁺): m/z 843.4 [M+H]⁺

### Example 338. Synthesis of 3-(5-(1-(2-(1-((1R,3S)-3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-3-methyl-2-oxo-2,3-dihydro-1H-benzo[d]imidazol-1-yl)piperidine-2,6-dione

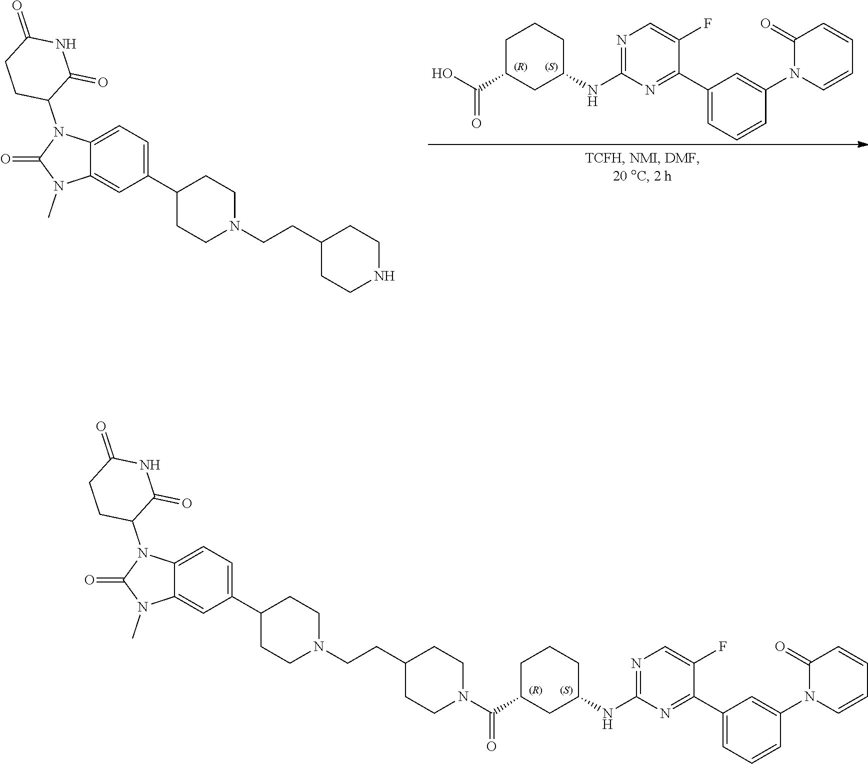

Synthesis of 3-(5-(1-(2-(1-((1R,3S)-3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-3-methyl-2-oxo-2,3-dihydro-1H-benzo[d]imidazol-1-yl)piperidine-2,6-dione (Compound 335). To a solution of (1R,3S)-3-((5-fluoro-4-(3-(2-oxopyridin-1(2H)-yl)phenyl)pyrimidin-2-yl)amino)cyclohexane-1-carboxylic acid (97.9 mg, 239 μmol, 0.800 eq) in DMF (3 mL) were added TCFH (101 mg, 359 μmol, 1.20 eq) and NMI (73.8 mg, 899 μmol, 71.6 μL, 3.00 eq) at 20 °C, then 3-(3-methyl-2-oxo-5-(1-(2-(piperidin-4-yl)ethyl)piperidin-4-yl)-2,3-dihydro-1H-benzo[d]imidazol-1-yl)piperidine-2,6-dione (170 mg, 299 μmol, 1.00 eq, TFA) was added to the mixture, the mixture was stirred at 20 °C for 2 h. LCMS showed 3-(3-methyl-2-oxo-5-(1-(2-(piperidin-4-yl)ethyl)piperidin-4-yl)-2,3-dihydro-1H-benzo[d]imidazol-1-yl)piperidine-2,6-dione was consumed completely and the desired target MS was detected. The mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 15%-50% B over 8.0 min) to give the title compound (Compound 335) (70.4 mg, 82.7 μmol, 27.6% yield, 99.1% purity) as a white solid. LCMS (ES⁺): m/z 844.4 [M+H]⁺

### Example 339. Synthesis of 3-(6-(1-(2-(1-((1R,3S)-3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 350)

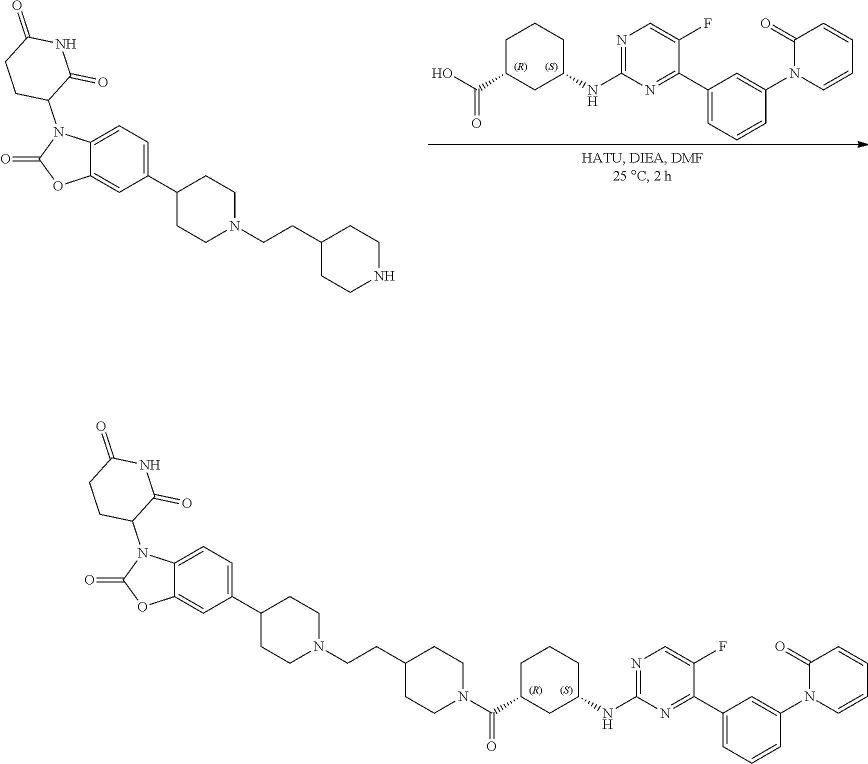

Synthesis of 3-(6-(1-(2-(1-((1R,3S)-3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 350). To a solution of (1R,3S)-3-((5-fluoro-4-(3-(2-oxopyridin-1(2H)-yl)phenyl)pyrimidin-2-yl)amino)cyclohexane-1-carboxylic acid (147 mg, 361 μmol, 1.00 eq) in DMF (4 mL) were added HATU (206 mg, 541 μmol, 1.50 eq) and DIEA (140 mg, 1.08 mmol, 188 μL, 3.00 eq) at 25 °C. Then 3-(2-oxo-6-(1-(2-(piperidin-4-yl)ethyl)piperidin-4-yl)benzo[d]oxazol-3 (2H)-yl)piperidine-2,6-dione (200 mg, 361 μmol, 1.00 eq, TFA) was added at 25 °C. The resulting mixture was stirred at 25 °C for 2 h. LCMS showed that 3-(2-oxo-6-(1-(2-(piperidin-4-yl)ethyl)piperidin-4-yl)benzo[d]oxazol-3 (2H)-yl)piperidine-2,6-dione was consumed completely and the desired mass was detected. The mixture was concentrated under reduced pressure. The residue was purified by prep-HPLC (column: Phenomenex Luna C18 75*30 mm*3 um; mobile phase: [1H₂O (0.2% FA)-ACN]; gradient: 15%-55% B over 8.0 min) to give the title compound (Compound 350) (45.8 mg, 54.9 μmol, 5.07% yield, 99.6% purity) as a yellow solid. LCMS (ES⁺): m/z 831.4 [M+H]⁺

### Example 340. Synthesis of 3-(6-(1-(2-(1-((1R,3S)-3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-1-methyl-1H-indazol-3-yl)piperidine-2,6-dione (Compound 351

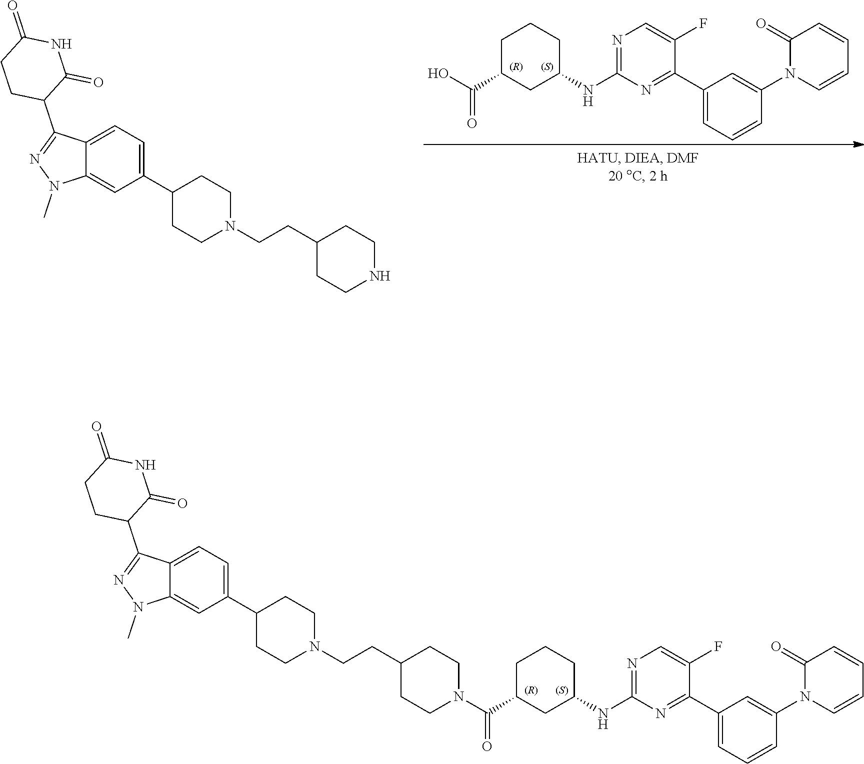

Synthesis of 3-(6-(1-(2-(1-((1R,3S)-3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-1-methyl-1H-indazol-3-yl)piperidine-2,6-dione (Compound 351). To a stirred solution of (1R,3S)-3-((5-fluoro-4-(3-(2-oxopyridin-1(2H)-yl)phenyl)pyrimidin-2-yl)amino)cyclohexane-1-carboxylic acid (222 mg, 544 μmol, 1.00 eq) and DIEA (212 mg, 1.64 mmol, 284 μL, 3.00 eq) in DMF (5 mL) were added HATU (310 mg, 816 μmol, 1.50 eq) and 3-(1-methyl-6-(1-(2-(piperidin-4-yl)ethyl)piperidin-4-yl)-1H-indazol-3-yl)piperidine-2,6-dione (300 mg, 544 μmol, 1.00 eq, TFA) at 20 °C. The resulting mixture was stirred at 20 °C for 2 h. LCMS showed that 3-(1-methyl-6-(1-(2-(piperidin-4-yl)ethyl)piperidin-4-yl)-1H-indazol-3-yl)piperidine-2,6-dione was consumed and the desired mass was detected. The mixture was concentrated under reduced pressure to give a residue. The residue was purified by prep-HPLC (column: Phenomenex Luna C18 80*30 mm*3 um; mobile phase: [H₂O (0.1% TFA)-ACN]; gradient: 25%-55% B over 8.0 min) to give the title compound (Compound 351) (6.70 mg, 7.34 μmol, 1.35% yield, 90.7% purity) as a yellow solid. LCMS (ES⁺): m/z 828.4 [M+H]⁺

### Example 341. Synthesis of 3-((3-fluoro-4-(1-(2-(1-((1S,3R)-3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)phenyl)amino)piperidine-2,6-dione (Compound 309)

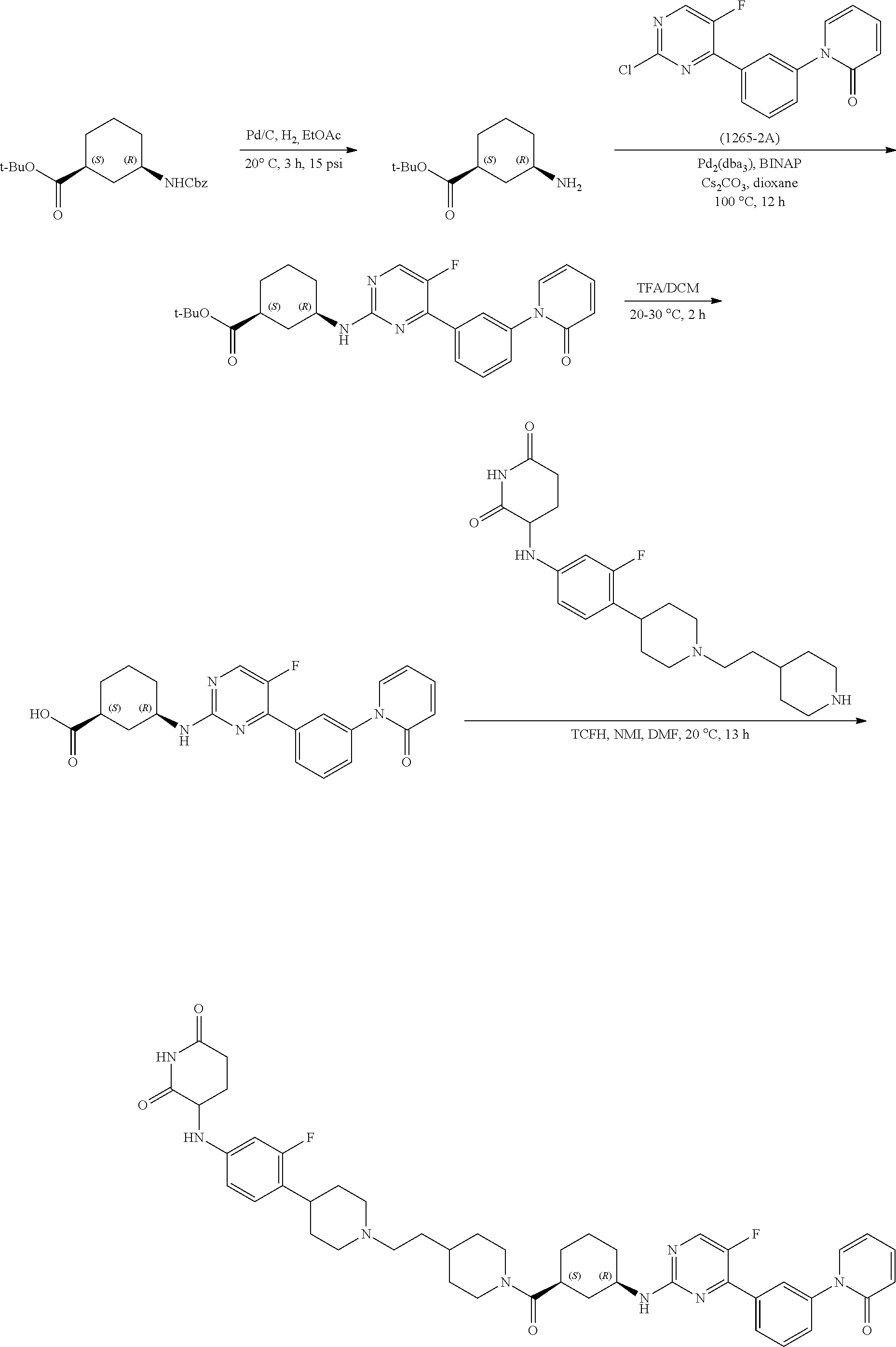

Synthesis of tert-butyl (1S,3R)-3-aminocyclohexane-1-carboxylate. To a solution of tert-butyl (1S,3R)-3-(((benzyloxy)carbonyl)amino)cyclohexane-1-carboxylate (1.00 g, 3.00 mmol, 1.00 eq) in EtOAc (10 mL) was added 10% Pd/C (1.00 g, 50% purity) at 20 °C. The mixture was degassed and purged with H₂ for three times, and then it was stirred at 20 °C for 3 h under H₂ (15 psi) atmosphere. LCMS showed tert-butyl (1S,3R)-3-(((benzyloxy)carbonyl)amino)cyclohexane-1-carboxylate was consumed and the desired mass was detected. The mixture was filtered and the filtrate was concentrated under reduced pressure to give the title compound (630 mg, crude) as a yellow oil. LCMS (ES⁺): m/z 200.2 [M+H]⁺.

Synthesis of tert-butyl (1S,3R)-3-((5-fluoro-4-(3-(2-oxopyridin-1(2H)-yl)phenyl)pyrimidin-2-yl)amino)cyclohexane-1-carboxylate. To a solution of 1-(3-(2-chloro-5-fluoropyrimidin-4-yl)phenyl)pyridin-2(1H)-one (513 mg, 1.70 mmol, 1.00 eq) and tert-butyl (1S,3R)-3-aminocyclohexane-1-carboxylate (610 mg, 3.06 mmol, 1.80 eq) in dioxane (20 mL) were added BINAP (95.3 mg, 153 μmol, 0.0900 eq), Cs₂CO₃ (1.66 g, 5.10 mmol, 3.00 eq) and Pd₂(dba)₃ (77.9 mg, 85.0 μmol, 0.0500 eq) at 20 °C under N₂. The mixture was stirred at 100 °C for 12 h under N₂ atmosphere. LCMS showed 1-(3-(2-chloro-5-fluoropyrimidin-4-yl)phenyl)pyridin-2(1H)-one was consumed and the desired mass was detected. To the mixture was added H₂O (20 mL) and the mixture was extracted with EtOAc (20 mL*3). The combined organic layers were washed with brine (30 mL*3), dried over Na₂SO₄, filtered and the filtrate was concentrated under reduced pressure to give a residue. The residue was purified by prep-TLC (SiO₂, PE/EtOAc=0/1) to give the title compound (700 mg, 1.51 mmol, 88.6% yield) as a yellow oil. LCMS (ES⁺): m/z 465.2 [M+H]⁺.

Synthesis of (1S,3R)-3-((5-fluoro-4-(3-(2-oxopyridin-1(2H)-yl)phenyl)pyrimidin-2-yl)amino)cyclohexane-1-carboxylic acid. A solution of tert-butyl (1S,3R)-3-((5-fluoro-4-(3-(2-oxopyridin-1(2H)-yl)phenyl)pyrimidin-2-yl)amino)cyclohexane-1-carboxylate (220 mg, 474 μmol, 1.00 eq) in DCM (4 mL) and TFA (0.8 mL) was stirred at 20 °C for 1 h. LCMS showed tert-butyl (1S,3R)-3-((5-fluoro-4-(3-(2-oxopyridin-1(2H)-yl)phenyl)pyrimidin-2-yl)amino)cyclohexane-1-carboxylate remained and the desired mass was detected. Then the mixture was stirred at 30 °C for another 1 h. LCMS showed tert-butyl (1S,3R)-3-((5-fluoro-4-(3-(2-oxopyridin-1(2H)-yl)phenyl)pyrimidin-2-yl)amino)cyclohexane-1-carboxylate was consumed completely and the desired mass was detected. The mixture was concentrated under reduced pressure to give the title compound (220 mg, crude) as yellow oil. LCMS (ES⁺): m/z 409.1 [M+H]⁺.

Synthesis of 3-((3-fluoro-4-(1-(2-(1-((1S,3R)-3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)phenyl)amino)piperidine-2,6-dione (Compound 309). To a solution of (1S,3R)-3-((5-fluoro-4-(3-(2-oxopyridin-1(2H)-yl)phenyl)pyrimidin-2-yl)amino)cyclohexane-1-carboxylic acid (200 mg, 490 μmol, 1.00 eq) in DMF (5 mL) were added TCFH (151 mg, 539 μmol, 1.10 eq) and NMI (125 mg, 1.52 mmol, 121 μL, 3.10 eq) at 20 °C, then 3-((3-fluoro-4-(1-(2-(piperidin-4-yl)ethyl)piperidin-4-yl)phenyl)amino)piperidine-2,6-dione (260 mg, 490 umol, 1.00 eq, TFA) was added to the mixture. The mixture was stirred at 20 °C for 1 h. LCMS showed (1S,3R)-3-((5-fluoro-4-(3-(2-oxopyridin-1(2H)-yl)phenyl)pyrimidin-2-yl)amino)cyclohexane-1-carboxylic acid remained and the desired mass was not detected. Then the mixture was stirred at 20 °C for another 12 h. LCMS showed (1S,3R)-3-((5-fluoro-4-(3-(2-oxopyridin-1(2H)-yl)phenyl)pyrimidin-2-yl)amino)cyclohexane-1-carboxylic acid was consumed completely and the desired mass was detected. To the mixture was added H₂O (5 mL) and the mixture was extracted with EtOAc (5 mL*3). The combined organic layers were washed with brine (5 mL*3), dried over Na₂SO₄, filtered and the filtrate was concentrated under reduced pressure to give a residue. The residue was purified by prep-HPLC (column: 3_Phenomenex Luna C18 75*30 mm*3 um; mobile phase: [H₂O (0.1% TFA)-ACN]; gradient: 18%-48% B over 8.0 min) to give the title compound (Compound 309) (19.8 mg, 22.4 gmol, 4.57% yield, 91.3% purity) as a yellow solid. LCMS (ES⁺): m/z 807.4 [M+H]⁺

### Example 342. Synthesis of 3-((4-(3,3-difluoro-1-(2-(1-((1S,3R)-3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-3-fluorophenyl)amino)piperidine-2,6-dione (Compound 311)

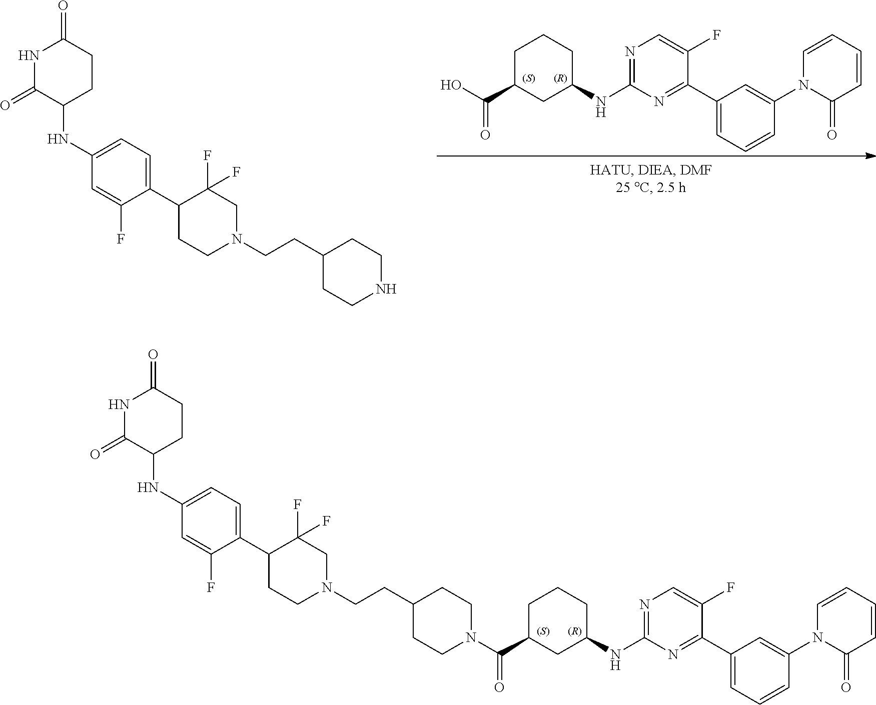

Synthesis of 3-((4-(3,3-difluoro-1-(2-(1-((1S,3R)-3-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-3-fluorophenyl)amino)piperidine-2,6-dione (Compound 311). To a solution of (1S,3R)-3-((5-fluoro-4-(3-(2-oxopyridin-1(2H)-yl)phenyl)pyrimidin-2-yl)amino)cyclohexane-1-carboxylic acid (72.1 mg, 177 μmol, 1.00 eq) in DMF (3 mL) were added HATU (101 mg, 265 umol, 1.50 eq) and DIEA (68.4 mg, 530 gmol, 92.2 μL, 3.00 eq) at 25 °C, the mixture was stirred at 25 °C for 0.5 h, and then 3-((4-(3,3-difluoro-1-(2-(piperidin-4-yl)ethyl)piperidin-4-yl)-3-fluorophenyl)amino)piperidine-2,6-dione (100 mg, 177 umol, 1.00 eq, TFA) was added to the mixture at 25 °C. The resulting mixture was stirred at 25 °C for 2 h. LCMS showed that 3-((4-(3,3-difluoro-1-(2-(piperidin-4-yl)ethyl)piperidin-4-yl)-3-fluorophenyl)amino)piperidine-2,6-dione was consumed completely the desired mass was detected. The mixture was filtered and the filtrate was concentrated under reduced pressure to give a residue. The residue was purified by prep-HPLC (column: Phenomenex Luna C18 80*30 mm*3 um; mobile phase: [H₂O (0.1% TFA)-ACN]; gradient: 25%-55% B over 8.0 min) to give the title compound (Compound 311) (5.90 mg, 6.71 μmol, 3.80% yield, 95.9% purity) as a yellow solid. LCMS (ES⁺): m/z 843.4 [M+H]⁺

### Example 343. Synthesis of trans-2-(4-((4-(1-(2,6-dioxopiperidin-3-yl)-3-methyl-2-oxo-2,3-dihydro-1H-benzo[d]imidazol-5-yl)piperidin-1-yl)methyl)piperidin-1-yl)-N-(4-((5-fluoro-4-(3-(2-oxopyridin-1(2H)-yl)phenyl)pyrimidin-2-yl)amino)cyclohexyl)acetamide (Compound 301)

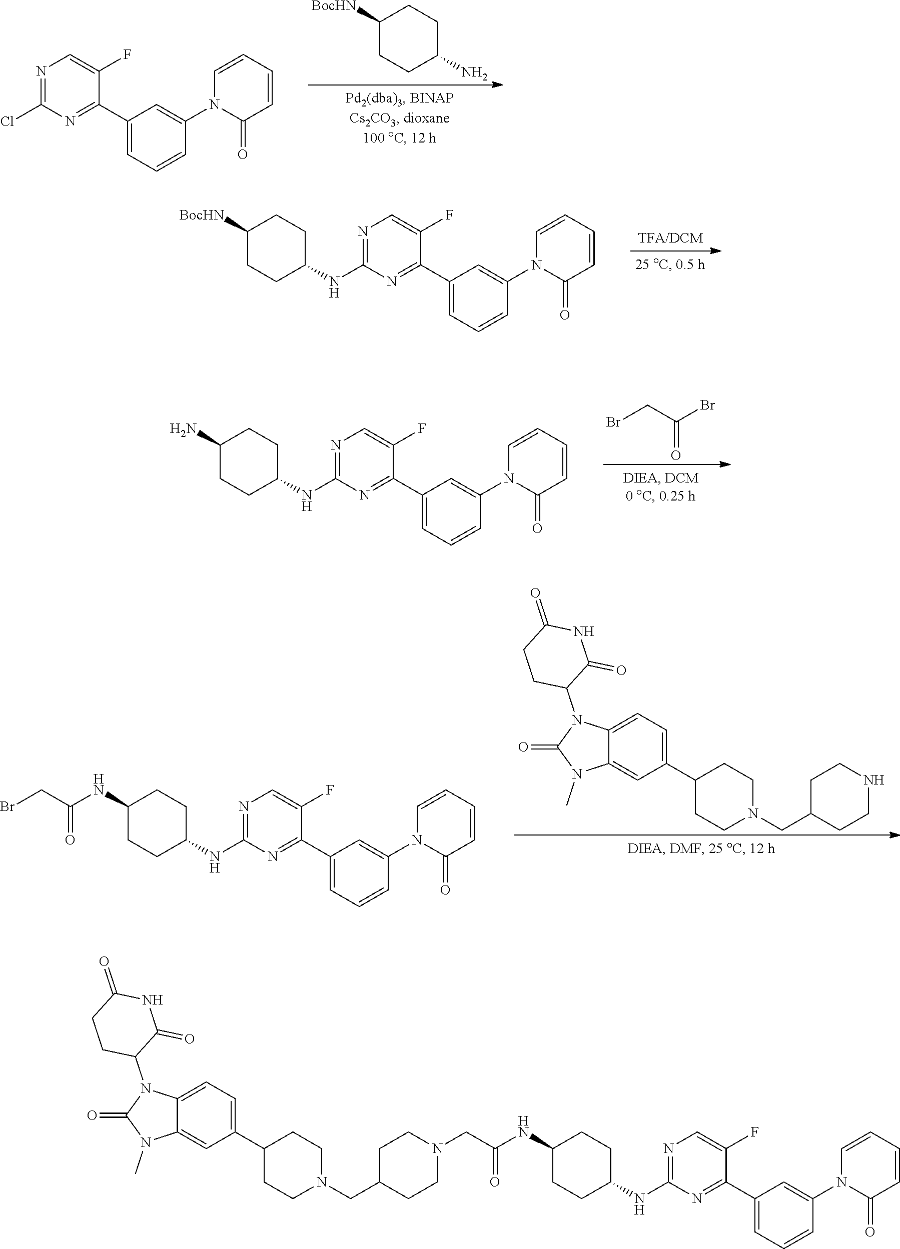

Synthesis of trans-tert-butyl (4-((5-fluoro-4-(3-(2-oxopyridin-1(2H)-yl)phenyl)pyrimidin-2-yl)amino)cyclohexyl)carbamate. To a solution of 1-(3-(2-chloro-5-fluoropyrimidin-4-yl)phenyl)pyridin-2(1H)-one (1.00 g, 3.31 mmol, 1.00 eq) and trans-tert-butyl (4-aminocyclohexyl)carbamate (1.07 g, 4.97 mmol, 1.50 eq) in dioxane (40 mL) were added BINAP (206 mg, 331 μmol, 0.100 eq), Cs₂CO₃ (3.24 g, 9.94 mmol, 3.00 eq) and Pd₂(dba)₃ (304 mg, 331 μmol, 0.100 eq) at 25 °C under N₂. The mixture was stirred at 100 °C for 12 h under N₂. LCMS showed 1-(3-(2-chloro-5-fluoropyrimidin-4-yl)phenyl)pyridin-2(1H)-one was consumed completely and the desired mass was detected. The mixture was diluted with H₂O (50 mL) and then extracted with EtOAc (30 mL*3). The combined organic layers were dried over Na₂SO₄, filtered and the filtrate was concentrated under reduced pressure to give a residue. The residue was purified by MPLC (SiO₂, Petroleum ether/Ethyl acetate=1/0 to 0/1) to give the title compound (1.08 g, 2.25 mmol, 68.0% yield) as a yellow oil. LCMS (ES⁺): m/z 480.3 [M+H]⁺.

Synthesis of trans-1-(3-(2-((4-aminocyclohexyl)amino)-5-fluoropyrimidin-4-yl)phenyl)pyridin-2(1H)-one. A solution of trans-tert-butyl (4-((5-fluoro-4-(3-(2-oxopyridin-1(2H)-yl)phenyl)pyrimidin-2-yl)amino)cyclohexyl)carbamate (700 mg, 1.46 mmol, 1.00 eq) in DCM (6 mL) and TFA (2 mL) was stirred at 25 °C for 0.5 h. LCMS showed trans-tert-butyl (4-((5-fluoro-4-(3-(2-oxopyridin-1(2H)-yl)phenyl)pyrimidin-2-yl)amino)cyclohexyl)carbamate was consumed completely and the desired mass was detected. The mixture was concentrated under reduced pressure to give the title compound (700 mg, crude, TFA) as a yellow oil. LCMS (ES⁺): m/z 380.2 [M+H]⁺.

Synthesis of trans-2-bromo-N-(4-((5-fluoro-4-(3-(2-oxopyridin-1(2H)-yl)phenyl)pyrimidin-2-yl)amino)cyclohexyl)acetamide. To a solution of trans-1-(3-(2-((4-aminocyclohexyl)amino)-5-fluoropyrimidin-4-yl)phenyl)pyridin-2(1H)-one (700 mg, 1.42 mmol, 1.00 eq, TFA) in DCM (10 mL) was added DIEA (550 mg, 4.26 mmol, 741 μL, 3.00 eq) at 25 °C, 2-bromoacetyl bromide (430 mg, 2.13 mmol, 185 μL, 1.50 eq) was added to the mixture at 0 °C. The mixture was stirred at 0 °C for 0.25 h. LCMS showed trans-1-(3-(2-((4-aminocyclohexyl)amino)-5-fluoropyrimidin-4-yl)phenyl)pyridin-2(1H)-one was consumed completely and the desired mass was detected. The mixture was concentrated under reduced pressure to give a residue. The residue was purified by MPLC (SiO₂, Petroleum ether/Ethyl acetate=1/0 to 0/1) to give the title compound (460 mg, 919 μmol, 64.8% yield) as a yellow solid. LCMS (ES⁺): m/z 500.2 [M+H]⁺.

Synthesis of trans-2-(4-((4-(1-(2,6-dioxopiperidin-3-yl)-3-methyl-2-oxo-2,3-dihydro-1H-benzo[d]imidazol-5-yl)piperidin-1-yl)methyl)piperidin-1-yl)-N-(4-((5-fluoro-4-(3-(2-oxopyridin-1(2H)-yl)phenyl)pyrimidin-2-yl)amino)cyclohexyl)acetamide (Compound 301). To a stirred solution of 3-(3-methyl-2-oxo-5-(1-(piperidin-4-ylmethyl)piperidin-4-yl)-2,3-dihydro-1H-benzo[d]imidazol-1-yl)piperidine-2,6-dione (120 mg, 217 μmol, 1.00 eq, TFA) in DMF (3 mL) were added DIEA (84.1 mg, 650 μmol, 113 μL, 3.00 eq) and trans-2-bromo-N-(4-((5-fluoro-4-(3-(2-oxopyridin-1(2H)-yl)phenyl)pyrimidin-2-yl)amino)cyclohexyl)acetamide (108 mg, 217 μmol, 1.00 eq) at 25 °C. The mixture was stirred at 25 °C for 12 h. LCMS showed trans-2-bromo-N-(4-((5-fluoro-4-(3-(2-oxopyridin-1(2H)-yl)phenyl)pyrimidin-2-yl)amino)cyclohexyl)acetamide was consumed completely and the desired mass was detected. The mixture was diluted with H₂O (5 mL) and then extracted with EtOAc (5 mL*3). The combined organic layers were dried over Na₂SO₄, filtered and the filtrate was concentrated under reduced pressure to give a residue. The residue was purified by prep-HPLC (column: Phenomenex Luna C18 100*30 mm*3 um; mobile phase: [H₂O (0.2% FA)-ACN]; gradient: 1%-25% B over 8.0 min) to give the title compound (Compound 301) (20.1 mg, 23.4 μmol, 10.8% yield, 99.8% purity) as a white solid. LCMS (ES⁺): m/z 859.4 [M+H]⁺

### Example 344. Synthesis of trans-2-(4-((4-(4-((2,6-dioxopiperidin-3-yl)amino)-2-fluorophenyl)piperidin-1-yl)methyl)piperidin-1-yl)-N-(4-((5-fluoro-4-(3-(2-oxopyridin-1(2H)-yl)phenyl)pyrimidin-2-yl)amino)cyclohexyl)acetamide (Compound 313)

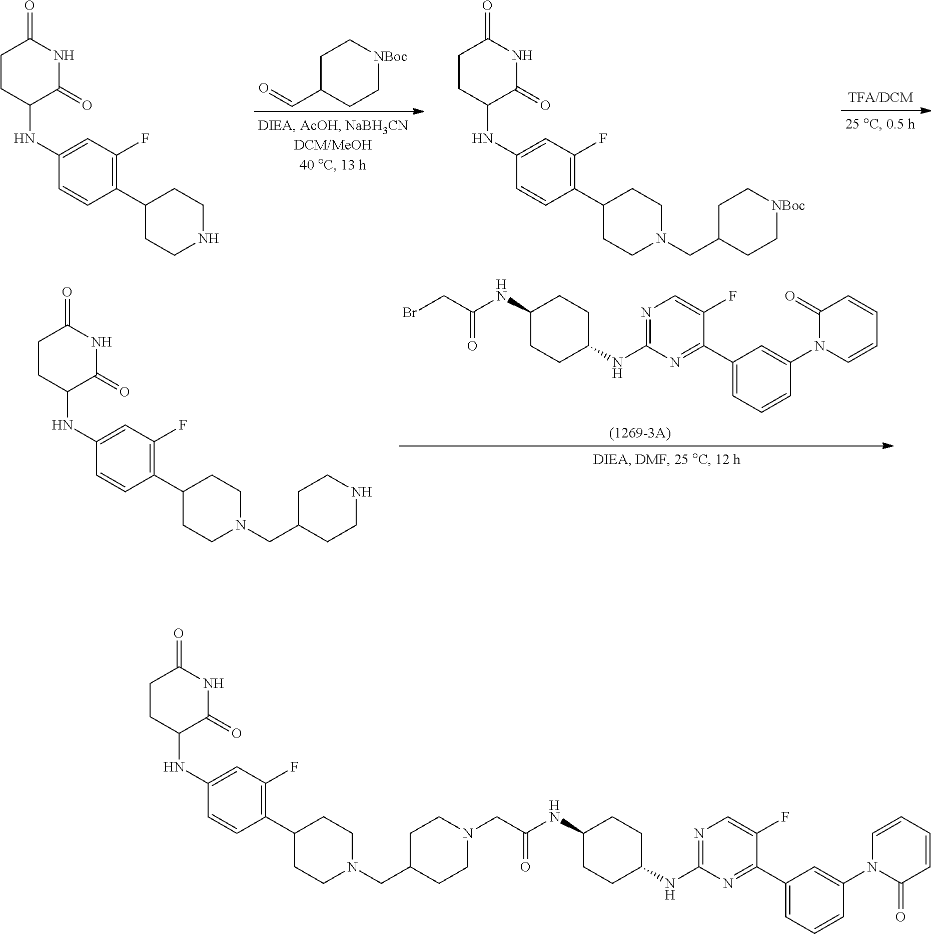

Synthesis of tert-butyl 4-((4-(4-((2,6-dioxopiperidin-3-yl)amino)-2-fluorophenyl)piperidin-1-yl)methyl)piperidine-1-carboxylate. To a solution of 3-((3-fluoro-4-(piperidin-4-yl)phenyl)amino)piperidine-2,6-dione (2.00 g, 4.77 mmol, 1.00 eq, TFA) in MeOH (5 mL) and DCM (25 mL) was added DIEA to adjust pH to 8 and the mixture was stirred at 25 °C for 0.5 h. AcOH (286 mg, 4.77 mmol, 273 μL, 1.00 eq) and tert-butyl 4-formylpiperidine-1-carboxylate (2.03 g, 9.54 mmol, 2.00 eq) was added to the mixture at 20 °C. pH was 5 now. The mixture was stirred at 40 °C for 0.5 h. Then NaBH₃CN (899 mg, 14.3 mmol, 3.00 eq) was added to the mixture at 40 °C. The mixture was stirred at 40 °C for 12 h. LCMS showed 3-((3-fluoro-4-(piperidin-4-yl)phenyl)amino)piperidine-2,6-dione was consumed completely and the desired mass was detected. The mixture was diluted with H₂O (20 mL) and then extracted with EtOAc (10 mL*3). The combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (870 mg, 1.73 mmol, 36.3% yield) as a yellow solid. LCMS (ES⁺): m/z 503.3 [M+H]⁺.

Synthesis of 3-((3-fluoro-4-(1-(piperidin-4-ylmethyl)piperidin-4-yl)phenyl)amino)piperidine-2,6-dione. A solution of tert-butyl 4-((4-(4-((2,6-dioxopiperidin-3-yl)amino)-2-fluorophenyl)piperidin-1-yl)methyl)piperidine-1-carboxylate (300 mg, 597 μmol, 1.00 eq) in DCM (1.5 mL) and TFA (0.5 mL) was stirred at 25 °C for 0.5 h. LCMS showed tert-butyl 4-((4-(4-((2,6-dioxopiperidin-3-yl)amino)-2-fluorophenyl)piperidin-1-yl)methyl)piperidine-1-carboxylate was consumed completely and the desired mass was detected. The mixture was concentrated under reduced pressure to give the title compound (300 mg, crude, TFA) as a yellow oil. LCMS (ES⁺): m/z 403.3 [M+H]⁺.

Synthesis of trans-2-(4-((4-(4-((2,6-dioxopiperidin-3-yl)amino)-2-fluorophenyl)piperidin-1-yl)methyl)piperidin-1-yl)-N-(4-((5-fluoro-4-(3-(2-oxopyridin-1(2H)-yl)phenyl)pyrimidin-2-yl)amino)cyclohexyl)acetamide (Compound 313). To a stirred solution of 3-((3-fluoro-4-(1-(piperidin-4-ylmethyl)piperidin-4-yl)phenyl)amino)piperidine-2,6-dione (150 mg, 290 μmol, 1.00 eq, TFA) in DMF (4 mL) were added DIEA (113 mg, 871 μmol, 152 μL, 3.00 eq) and trans-2-bromo-N-(4-((5-fluoro-4-(3-(2-oxopyridin-1(2H)-yl)phenyl)pyrimidin-2-yl)amino)cyclohexyl)acetamide (145 mg, 290 μmol, 1.00 eq) at 25 °C, the mixture was stirred at 25 °C for 12 h. LCMS showed 3-((3-fluoro-4-(1-(piperidin-4-ylmethyl)piperidin-4-yl)phenyl)amino)piperidine-2,6-dione was consumed completely and the desired mass was detected. The mixture was diluted with H₂O (5 mL) and then extracted with EtOAc (5 mL*3). The combined organic layers were dried over Na₂SO₄, filtered and the filtrate was concentrated under reduced pressure to give a residue. The residue was purified by prep-HPLC (column: Phenomenex luna C18 100*40 mm*3 um; mobile phase: [H₂O (0.2% FA)-ACN]; gradient: 1%-40% B over 8.0 min) to give the title compound (Compound 313) (32.4 mg, 39.3 μmol, 13.5% yield, 99.8% purity) as a white solid. LCMS (ES⁺): m/z 822.3 [M+H]⁺

### Example 345. Synthesis of trans-3-(4-(4-((2,6-dioxopiperidin-3-yl)amino)-2-fluorophenyl)-[1,4′-bipiperidin]-1′-yl)-N-(4-((5-fluoro-4-(3-(2-oxopyridin-1(2H)-yl)phenyl)pyrimidin-2-yl)amino)cyclohexyl)propanamide (Compound 328)

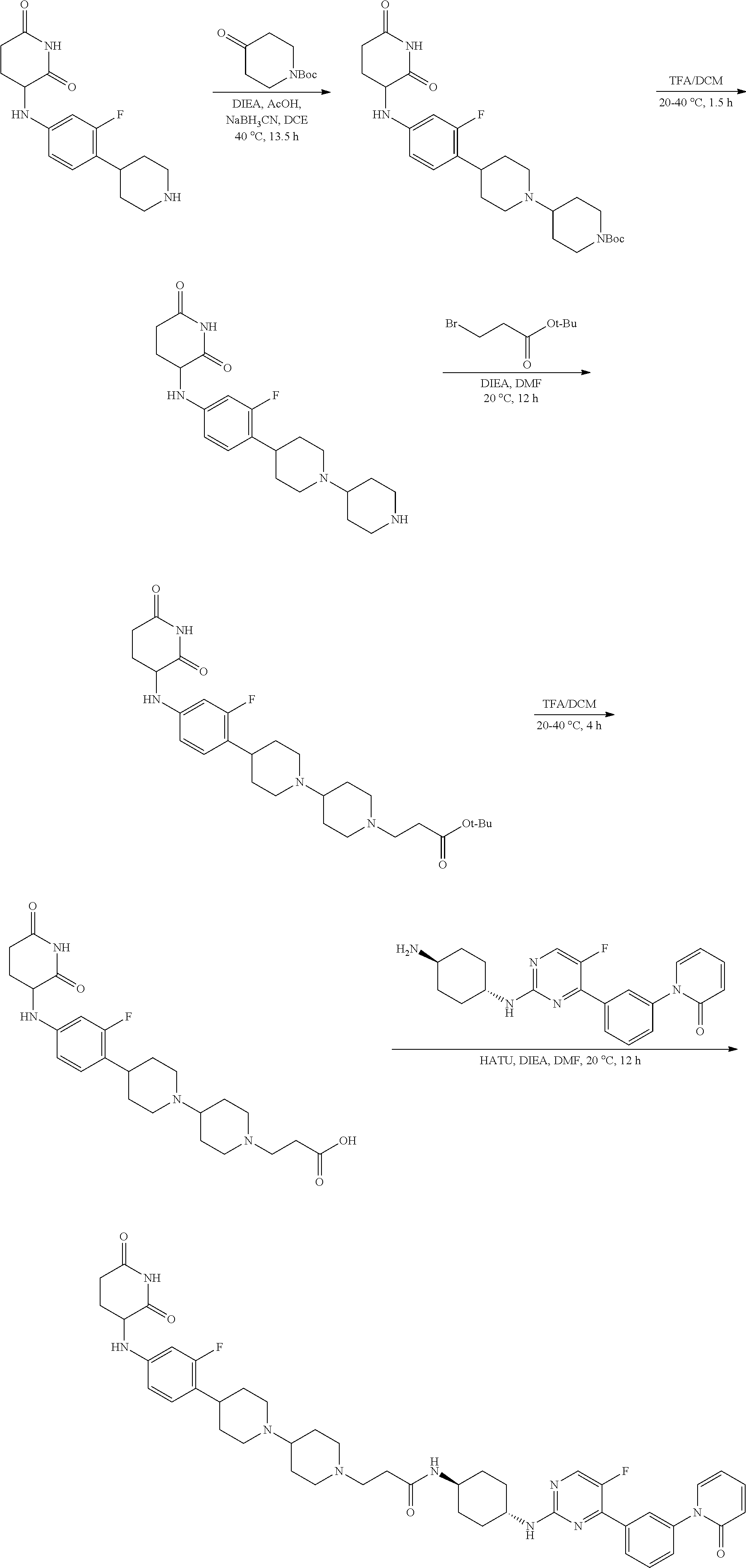

Synthesis of tert-butyl 4-(4-((2,6-dioxopiperidin-3-yl)amino)-2-fluorophenyl)-[1,4′-bipiperidine]-1′-carboxylate. To a solution of 3-((3-fluoro-4-(piperidin-4-yl)phenyl)amino)piperidine-2,6-dione (2.00 g, 4.77 mmol, 1.00 eq, TFA) in DCE (20 mL) was added DIEA to adjust pH to 8 at 20 °C. The mixture was stirred at 20 °C for 1 h. To the mixture was added AcOH (286 mg, 4.77 mmol, 273 μL, 1.00 eq) and tert-butyl 4-oxopiperidine-1-carboxylate (7.60 g, 38.2 mmol, 8.00 eq) at 20 °C, pH was 5 now. The mixture was stirred at 40 °C for 0.5 h. Then NaBH₃CN (899 mg, 14.3 mmol, 3.00 eq) was added to the mixture at 20 °C and the resulting mixture was stirred at 40 °C for 12 h. LCMS showed 3-((3-fluoro-4-(piperidin-4-yl)phenyl)amino)piperidine-2,6-dione was consumed partly and the desired mass was detected. To the mixture was added H₂O (20 mL) and the mixture was extracted with EtOAc (20 mL*3). The combined organic layers were washed with brine (20 mL*3), dried over Na₂SO₄, filtered and the filtrate was concentrated under reduced pressure to give a residue. The residue was purified by MPLC (SiO₂, DCM/MeOH=1/0 to 5/1) to give the title compound (700 mg, 1.43 mmol, 30.0% yield) as a yellow oil. LCMS (ES⁺): m/z 489.3 [M+H]⁺.

Synthesis of 3-((4-([1,4′-bipiperidin]-4-yl)-3-fluorophenyl)amino)piperidine-2,6-dione. A solution of tert-butyl 4-(4-((2,6-dioxopiperidin-3-yl)amino)-2-fluorophenyl)-[1,4′-bipiperidine]-1′-carboxylate (500 mg, 1.02 mmol, 1.00 eq) in DCM (2.5 mL) and TFA (0.5 mL) was stirred at 20 °C for 0.5 h. LCMS showed tert-butyl 4-(4-((2,6-dioxopiperidin-3-yl)amino)-2-fluorophenyl)-[1,4′-bipiperidine]-1′-carboxylate remained and the desired mass was detected. Then the mixture was stirred at 40 °C for another 1 h. LCMS showed tert-butyl 4-(4-((2,6-dioxopiperidin-3-yl)amino)-2-fluorophenyl)-[1,4′-bipiperidine]-1′-carboxylate was consumed and the desired mass was detected. The mixture was concentrated under reduced pressure to give the title compound (500 mg, crude, TFA) as a yellow oil. LCMS (ES⁺): m/z 389.1 [M+H]⁺.

Synthesis of tert-butyl 3-(4-(4-((2,6-dioxopiperidin-3-yl)amino)-2-fluorophenyl)-[1,4′-bipiperidin]-1′-yl)propanoate. To a solution of 3-((4-([1,4′-bipiperidin]-4-yl)-3-fluorophenyl)amino)piperidine-2,6-dione (500 mg, 995 μmol, 1.00 eq, TFA) in DMF (2 mL) were added DIEA (386 mg, 2.99 mmol, 520 μL, 3.00 eq) and tert-butyl 3-bromopropanoate (416 mg, 1.99 mmol, 332 μL, 2.00 eq) at 20 °C. The mixture was stirred at 20 °C for 12 h. LCMS showed 3-((4-([1,4′-bipiperidin]-4-yl)-3-fluorophenyl)amino)piperidine-2,6-dione remained and the desired mass was detected. The mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 1%-35% B over 8.0 min) to give the title compound (50.0 mg, 61.8 μmol, 6.21% yield, 63.9% purity) as a yellow solid. LCMS (ES⁺): m/z 517.5 [M+H]⁺.

Synthesis of 3-(4-(4-((2,6-dioxopiperidin-3-yl)amino)-2-fluorophenyl)-[1,4′-bipiperidin]-1′-yl)propanoic acid. A solution of tert-butyl 3-(4-(4-((2,6-dioxopiperidin-3-yl)amino)-2-fluorophenyl)-[1,4′-bipiperidin]-1′-yl)propanoate (50.0 mg, 96.8 μmol, 1.00 eq) in DCM (2 mL) and TFA (0.4 mL) was stirred at 20 °C for 2 h. LCMS showed tert-butyl 3-(4-(4-((2,6-dioxopiperidin-3-yl)amino)-2-fluorophenyl)-[1,4′-bipiperidin]-1′-yl)propanoate remained and the desired mass was detected. Then the mixture was stirred at 40 °C for another 2 h. LCMS showed tert-butyl 3-(4-(4-((2,6-dioxopiperidin-3-yl)amino)-2-fluorophenyl)-[1,4′-bipiperidin]-1′-yl)propanoate was consumed and the desired mass was detected. The mixture was concentrated under reduced pressure to give the title compound (50.0 mg, crude) as a yellow oil. LCMS (ES⁺): m/z 461.2 [M+H]⁺.

Synthesis of trans-3-(4-(4-((2,6-dioxopiperidin-3-yl)amino)-2-fluorophenyl)-[1,4′-bipiperidin]-1′-yl)-N-(4-((5-fluoro-4-(3-(2-oxopyridin-1(2H)-yl)phenyl)pyrimidin-2-yl)amino)cyclohexyl)propanamide (Compound 328). To a solution of 3-(4-(4-((2,6-dioxopiperidin-3-yl)amino)-2-fluorophenyl)-[1,4′-bipiperidin]-1′-yl)propanoic acid (50.0 mg, 109 μmol, 1.00 eq) in DMF (2 mL) were added HATU (28.9 mg, 76.0 μmol, 0.700 eq) and DIEA (28.1 mg, 217 μmol, 37.8 μL, 2.00 eq) at 20 °C, then trans-1-(3-(2-((4-aminocyclohexyl)amino)-5-fluoropyrimidin-4-yl)phenyl)pyridin-2(1H)-one (31.6 mg, 76.0 μmol, 0.700 eq, HCl) was added to the mixture at 20 °C. The mixture was stirred at 20 °C for 12 h. LCMS showed 3-(4-(4-((2,6-dioxopiperidin-3-yl)amino)-2-fluorophenyl)-[1,4′-bipiperidin]-1′-yl)propanoic acid was consumed and the desired mass was detected. To the mixture was added H₂O (5 mL) and the mixture was extracted with EtOAc (5 mL*3). The combined organic layers were washed with brine (5 mL*3), dried over Na₂SO₄, filtered and the filtrate was concentrated under reduced pressure to residue. The residue was purified by prep-HPLC (column: Phenomenex Luna C18 100*30 mm*3 um; mobile phase: [H₂O (0.2% FA)-ACN]; gradient: 5%-35% B over 8.0 min) to give the title compound (Compound 328) (8.20 mg, 9.75 μmol, 8.98% yield, 97.8% purity, FA) as a white solid. LCMS (ES⁺): m/z 822.4 [M+H]⁺

### Example 346. Synthesis of trans-2-(4-((4-(3-(2,6-dioxopiperidin-3-yl)-2-oxo-2,3-dihydrobenzo[d]oxazol-6-yl)piperidin-1-yl)methyl)piperidin-1-yl)-N-(4-((5-fluoro-4-(3-(2-oxopyridin-1(2H)-yl)phenyl)pyrimidin-2-yl)amino)cyclohexyl)acetamide (Compound 302)

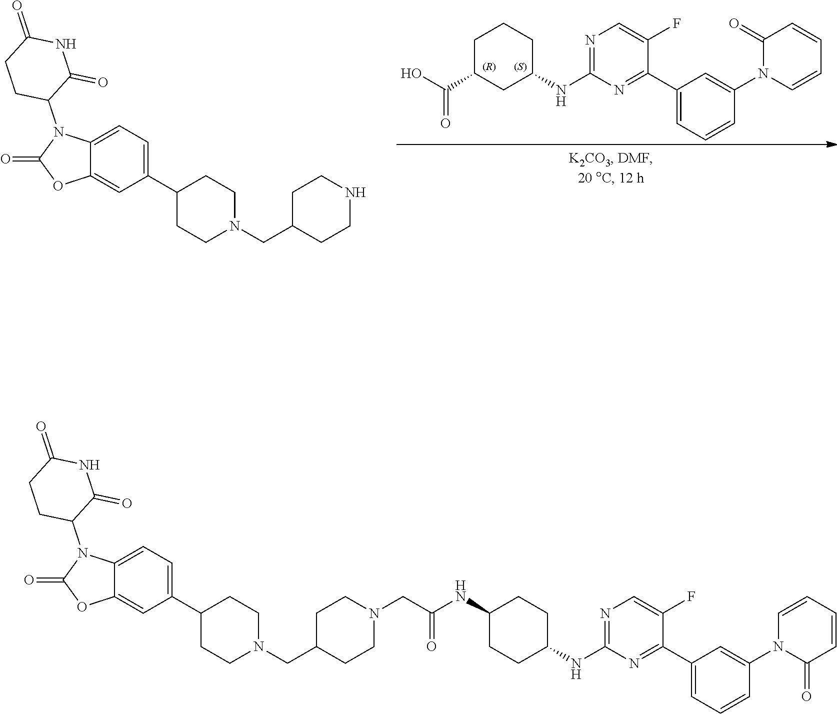

Synthesis of trans-2-(4-((4-(3-(2,6-dioxopiperidin-3-yl)-2-oxo-2,3-dihydrobenzo[d]oxazol-6-yl)piperidin-1-yl)methyl)piperidin-1-yl)-N-(4-((5-fluoro-4-(3-(2-oxopyridin-1(2H)-yl)phenyl)pyrimidin-2-yl)amino)cyclohexyl)acetamide (Compound 302). To a solution of 3-(2-oxo-6-(1-(piperidin-4-ylmethyl)piperidin-4-yl)benzo[d]oxazol-3 (2H)-yl)piperidine-2,6-dione (100 mg, 216 μmol, 1.00 eq, HCl) in DMF (4 mL) were added K₂CO₃ (149 mg, 1.08 mmol, 5.00 eq) and trans-2-bromo-N-[4-[[5-fluoro-4-[3-(2-oxo-1-pyridyl)phenyl]pyrimidin-2-yl]amino]cyclohexyl]acetamide (108 mg, 216 μmol, 1.00 eq) at 20 °C. The mixture was stirred at 20 °C for 2 h. LCMS showed 3-(2-oxo-6-(1-(piperidin-4-ylmethyl)piperidin-4-yl)benzo[d]oxazol-3 (2H)-yl)piperidine-2,6-dione was remaining and the desired mass was detected. The mixture was stirred at 20 °C for another 10 h. LCMS showed 3-(2-oxo-6-(1-(piperidin-4-ylmethyl)piperidin-4-yl)benzo[d]oxazol-3 (2H)-yl)piperidine-2,6-dione was consumed completely and the desired mass was detected. The reaction mixture was filtered and the filtrate was concentrated under reduced pressure to give a residue. The residue was purified by prep-HPLC (column: Phenomenex luna C18 100*40 mm*3 um; mobile phase: [H₂O (0.2% FA)-ACN]; gradient: 1%-35% B over 8.0 min) to give the title compound (Compound 302) (13.3 mg, 14.8 μmol, 6.83% yield, 99.0% purity, FA) as a white solid. LCMS (ES⁺): m/z 846.4 [M+H]⁺

### Example 347. Synthesis of trans-2-(4-((4-(3-(2,6-dioxopiperidin-3-yl)-1-methyl-1H-indazol-6-yl)piperidin-1-yl)methyl)piperidin-1-yl)-N-(4-((5-fluoro-4-(3-(2-oxopyridin-1(2H)-yl)phenyl)pyrimidin-2-yl)amino)cyclohexyl)acetamide (Compound 304)

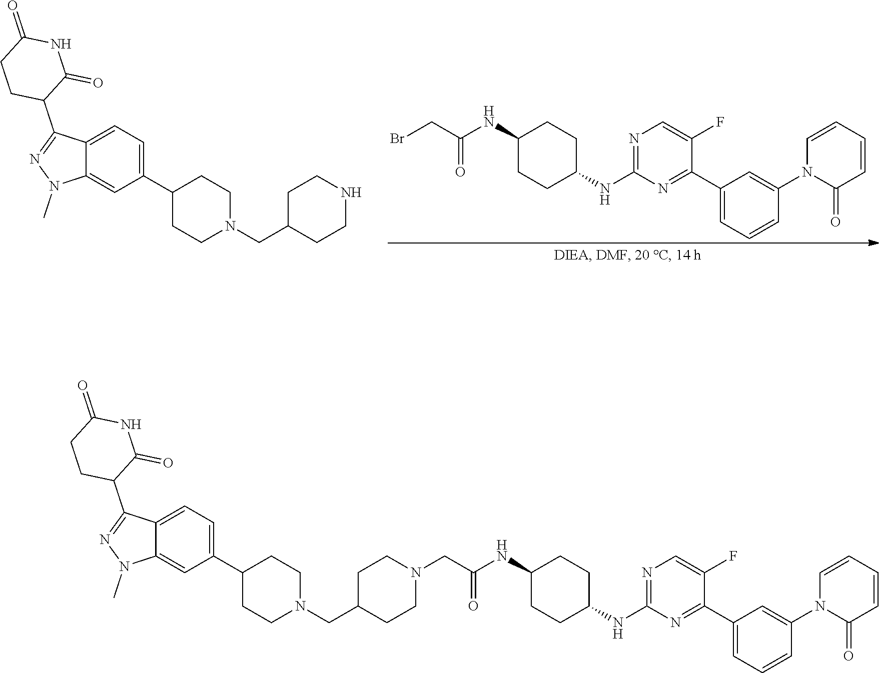 [SMILES: Cn1nc(C2CCC(=O)NC2=O)c2ccc(C3CCN(CC4CCNCC4)CC3)cc21 ; O=C(CBr)N[C@H]1CC[C@H](Nc2ncc(F)c(-c3cccc(-n4ccccc4=O)c3)n2)CC1 ; Cn1nc(C2CCC(=O)NC2=O)c2ccc(C3CCN(CC4CCN(CC(=O)N[C@H]5CC[C@H](Nc6ncc(F)c(-c7cccc(-n8ccccc8=O)c7)n6)CC5)CC4)CC3)cc21]

Synthesis of trans-2-(4-((4-(3-(2,6-dioxopiperidin-3-yl)-1-methyl-1H-indazol-6-yl)piperidin-1-yl)methyl)piperidin-1-yl)-N-(4-((5-fluoro-4-(3-(2-oxopyridin-1(2H)-yl)phenyl)pyrimidin-2-yl)amino)cyclohexyl)acetamide (Compound 304). To a solution of 3-(1-methyl-6-(1-(piperidin-4-ylmethyl)piperidin-4-yl)-1H-indazol-3-yl)piperidine-2,6-dione (118 mg, 220 μmol, 1.00 eq, TFA) in DMF (3 mL) were added DIEA (85.2 mg, 660 μmol, 115 μL, 3.00 eq) and trans-2-bromo-N-((1r,4r)-4-((5-fluoro-4-(3-(2-oxopyridin-1(2H)-yl)phenyl)pyrimidin-2-yl)amino)cyclohexyl)acetamide (110 mg, 220 μmol, 1.00 eq) at 20 °C. Then the mixture was stirred at 20 °C for 2 h. LCMS showed 3-(1-methyl-6-(1-(piperidin-4-ylmethyl)piperidin-4-yl)-1H-indazol-3-yl)piperidine-2,6-dione remained and the desired target MS was detected. Then the mixture was stirred at 20 °C for another 12 h, LCMS showed 3-(1-methyl-6-(1-(piperidin-4-ylmethyl)piperidin-4-yl)-1H-indazol-3-yl)piperidine-2,6-dione was consumed completely and the desired target MS was detected. The mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 10%-40% B over 8.0 min) to give the title compound (Compound 304) (5.10 mg, 5.56 μmol, 2.53% yield, 92.0% purity) as a gray solid. LCMS (ES⁺): m/z 843.4 [M+H]⁺

### Example 348. Synthesis of trans-1-[2-[1-[3-(2,6-dioxo-3-piperidyl)-2-oxo-1,3-benzoxazol-6-yl]-4-piperidyl]ethyl]-N-[4-[[5-fluoro-4-[3-(2-oxo-1-pyridyl)phenyl]pyrimidin-2-yl]amino]cyclohexyl]piperidine-4-carboxamide (Compound 329)

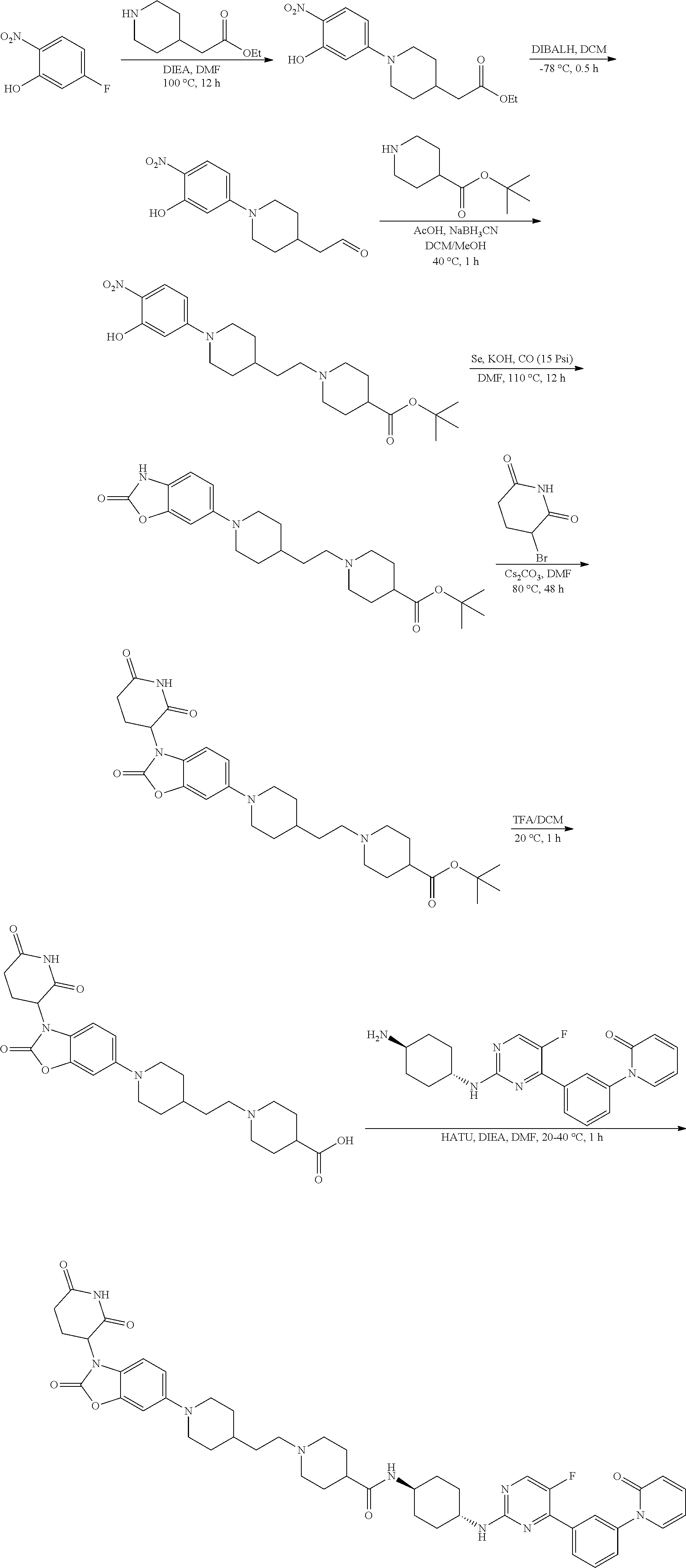

Synthesis of ethyl 2-[i-(3-hydroxy-4-nitro-phenyl)-4-piperidyl]acetate. To a solution of 5-fluoro-2-nitro-phenol (4.59 g, 29.2 mmol, 1.00 eq) and ethyl 2-(4-piperidyl)acetate (5.00 g, 29.2 mmol, 1.00 eq) in DMF (30 mL) was added DIEA (4.53 g, 35.0 mmol, 6.10 mL, 1.20 eq) at 20 °C, the mixture was stirred at 100 °C for 12 h. LCMS showed the desired mass was detected. The mixture was added to H₂O (100 mL) and filtered. The filter cake was dried under reduced pressure to give the title compound (7.80 g, crude) as a yellow solid. LCMS (ES⁺): m/z 309.1 [M+H]⁺.

Synthesis of 2-[1-(3-hydroxy-4-nitro-phenyl)-4-piperidyl]acetaldehyde. To a solution of ethyl 2-[1-(3-hydroxy-4-nitro-phenyl)-4-piperidyl]acetate (1.00 g, 3.24 mmol, 1.00 eq) in DCM (20 mL) was added DIBALH (3.89 mL, 1.20 eq, 1 M in Tol.) at −78 °C under N₂, the mixture was stirred at −78 °C for 0.5 h under N₂ atmosphere. LCMS showed ethyl 2-[1-(3-hydroxy-4-nitro-phenyl)-4-piperidyl]acetate was consumed completely and the desired mass was detected. The mixture was quenched by the addition of methanol (30 mL) dropwise at −30 °C and stirred at −30 °C for 0.5 h. Then the mixture was poured into saturated aqueous sodium potassium tartrate solution (100 mL), the resulting mixture was stirred vigorously at 20 °C for 1 h and extracted with DCM (50 mL*3). The combined organic phases were dried over Na₂SO₄, filtered and the filtrate was concentrated under reduced pressure. The mixture was purified by prep-TLC (SiO₂, PE/EtOAc=1/1) to give the title compound (525 mg, 1.99 mmol, 61.3% yield) as a yellow solid. LCMS (ES⁺): m/z 265.0 [M+H]⁺.

Synthesis of tert-butyl 1-[2-[1-(3-hydroxy-4-nitro-phenyl)-4-piperidyl]ethyl]piperidine-4-carboxylate. To a solution of 2-[1-(3-hydroxy-4-nitro-phenyl)-4-piperidyl]acetaldehyde (525 mg, 1.99 mmol, 1.00 eq) and tert-butyl piperidine-4-carboxylate (368 mg, 1.99 mmol, 1.00 eq) in DCM (10 mL) and MeOH (10 mL) was added AcOH (119 mg, 1.99 mmol, 114 μL, 1.00 eq) at 20 °C, the mixture was stirred at 40 °C for 0.5 h. Then NaBH₃CN (187 mg, 2.98 mmol, 1.50 eq) was added at 40 °C, the mixture was stirred at 40 °C for 0.5 h. LCMS showed 2-[1-(3-hydroxy-4-nitro-phenyl)-4-piperidyl]acetaldehyde was consumed completely and the desired mass was detected. To the mixture was added H₂O (30 mL) and the mixture was extracted with EtOAc (10 mL*3). The combined organic layers were washed with brine (10 mL*3), dried over Na₂SO₄, filtered and the filtrate was concentrated under reduced pressure to give the title compound (900 mg, crude) as a yellow solid. LCMS (ES⁺): m/z 434.2 [M+H]⁺.

Synthesis of tert-butyl 1-[2-[1-(2-oxo-3H-1,3-benzoxazol-6-yl)-4-piperidyl]ethyl]piperidine-4-carboxylate. To a solution of tert-butyl 1-[2-[1-(3-hydroxy-4-nitro-phenyl)-4-piperidyl]ethyl]piperidine-4-carboxylate (900 mg, 2.08 mmol, 1.00 eq) in DMF (10 mL) were added Se (164 mg, 2.08 mmol, 165 μL, 1.00 eq) and KOH (233 mg, 4.15 mmol, 2.00 eq) at 20 °C, the mixture was degassed and purged with CO for three times. The mixture was stirred at 110 °C for 12 h under CO (15 psi). LCMS showed tert-butyl 1-[2-[1-(3-hydroxy-4-nitro-phenyl)-4-piperidyl]ethyl]piperidine-4-carboxylate was consumed completely and the desired mass was detected. To the mixture was added H₂O (10 mL) and the mixture was extracted with EtOAc (10 mL*3). The combined organic layers were washed with brine (10 mL*3), dried over Na₂SO₄, filtered and the filtrate was concentrated under reduced pressure to give the title compound (900 mg, crude) as a yellow solid. LCMS (ES⁺): m/z 430.3 [M+H]⁺.

Synthesis of tert-butyl 1-[2-[1-[3-(2,6-dioxo-3-piperidyl)-2-oxo-1,3-benzoxazol-6-yl]-4-piperidyl]ethyl]piperidine-4-carboxylate. To a solution of tert-butyl 1-[2-[1-(2-oxo-3H-1,3-benzoxazol-6-yl)-4-piperidyl]ethyl]piperidine-4-carboxylate (800 mg, 1.86 mmol, 1.00 eq) in DMF (20 mL) was added Cs₂CO₃ (1.82 g, 5.59 mmol, 3.00 eq) at 20 °C. Then 3-bromopiperidine-2,6-dione (1.07 g, 5.59 mmol, 3.00 eq) was added at 20 °C, the mixture was stirred at 80 °C for 48 h. LCMS showed tert-butyl 1-[2-[1-(2-oxo-3H-1,3-benzoxazol-6-yl)-4-piperidyl]ethyl]piperidine-4-carboxylate was consumed completely that the desired mass was detected. To the mixture was added H₂O (50 mL) and the mixture was extracted with EtOAc (30 mL*3). The combined organic layers were washed with brine (20 mL*3), dried over Na₂SO₄, filtered and the filtrate was concentrated under reduced pressure to give a residue. The residue was purified by prep-TLC (SiO₂, PE/THF=1/1) to give the title compound (100 mg, 185 μmol, 9.93% yield) as a green oil liquid. LCMS (ES⁺): m/z 541.3 [M+H]⁺.

Synthesis of 1-[2-[1-[3-(2,6-dioxo-3-piperidyl)-2-oxo-1,3-benzoxazol-6-yl]-4-piperidyl]ethyl]piperidine-4-carboxylic acid. To a solution of tert-butyl 1-[2-[1-[3-(2,6-dioxo-3-piperidyl)-2-oxo-1,3-benzoxazol-6-yl]-4-piperidyl]ethyl]piperidine-4-carboxylate (90.0 mg, 166 μmol, 1.00 eq) in DCM (0.8 mL) was added TFA (0.2 mL) at 20 °C, the mixture was stirred at 20 °C for 1 h. LCMS showed that the desired mass was detected. The mixture was concentrated under reduced pressure to give the title compound (100 mg, crude) as a yellow oil liquid. LCMS (ES⁺): m/z 485.3 [M+H]⁺.

Synthesis of trans-1-[2-[1-[3-(2,6-dioxo-3-piperidyl)-2-oxo-1,3-benzoxazol-6-yl]-4-piperidyl]ethyl]-N-[4-[[5-fluoro-4-[3-(2-oxo-1-pyridyl)phenyl]pyrimidin-2-yl]amino]cyclohexyl]piperidine-4-carboxamide (Compound 329). To a solution of 1-[2-[1-[3-(2,6-dioxo-3-piperidyl)-2-oxo-1,3-benzoxazol-6-yl]-4-piperidyl]ethyl]piperidine-4-carboxylic acid (44.0 mg, 90.8 μmol, 1.00 eq) in DMF (2 mL) were added DIEA (58.8 mg, 455 μmol, 79.0 μL, 5.00 eq) and HATU (38.0 mg, 100.0 μmol, 1.10 eq) at 20 °C, the mixture was stirred at 20 °C for 0.5 h. Then trans-1-[3-[2-[(4-aminocyclohexyl)amino]-5-fluoro-pyrimidin-4-yl]phenyl]pyridin-2-one (44.8 mg, 90.8 μmol, 1.00 eq, TFA) was added at 20 °C, the mixture was stirred at 40 °C for 0.5 h. LCMS showed 1-[2-[1-[3-(2,6-dioxo-3-piperidyl)-2-oxo-1,3-benzoxazol-6-yl]-4-piperidyl]ethyl]piperidine-4-carboxylic acid was consumed completely and the desired mass was detected. To the mixture was added H₂O (3 mL) and the mixture was extracted with EtOAc (3 mL*3). The combined organic layers were washed with brine (3 mL*3), dried over Na₂SO₄, filtered and the filtrate was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 20%-50% B over 8.0 min) to give the title compound (Compound 329) (4.0 mg, 4.73 umol, 5.21% yield, 100% purity) as a white solid. LCMS (ES⁺): m/z 846.4 [M+H]⁺

### Example 349. Synthesis of trans-1-[2-[1-[4-[(2,6-dioxo-3-piperidyl)amino]-2-fluoro-phenyl]-4-piperidyl]ethyl]-N-[4-[[5-fluoro-4-[3-(2-oxo-1-pyridyl)phenyl]pyrimidin-2-yl]amino]cyclohexyl]piperidine-4-carboxamide (Compound 316)

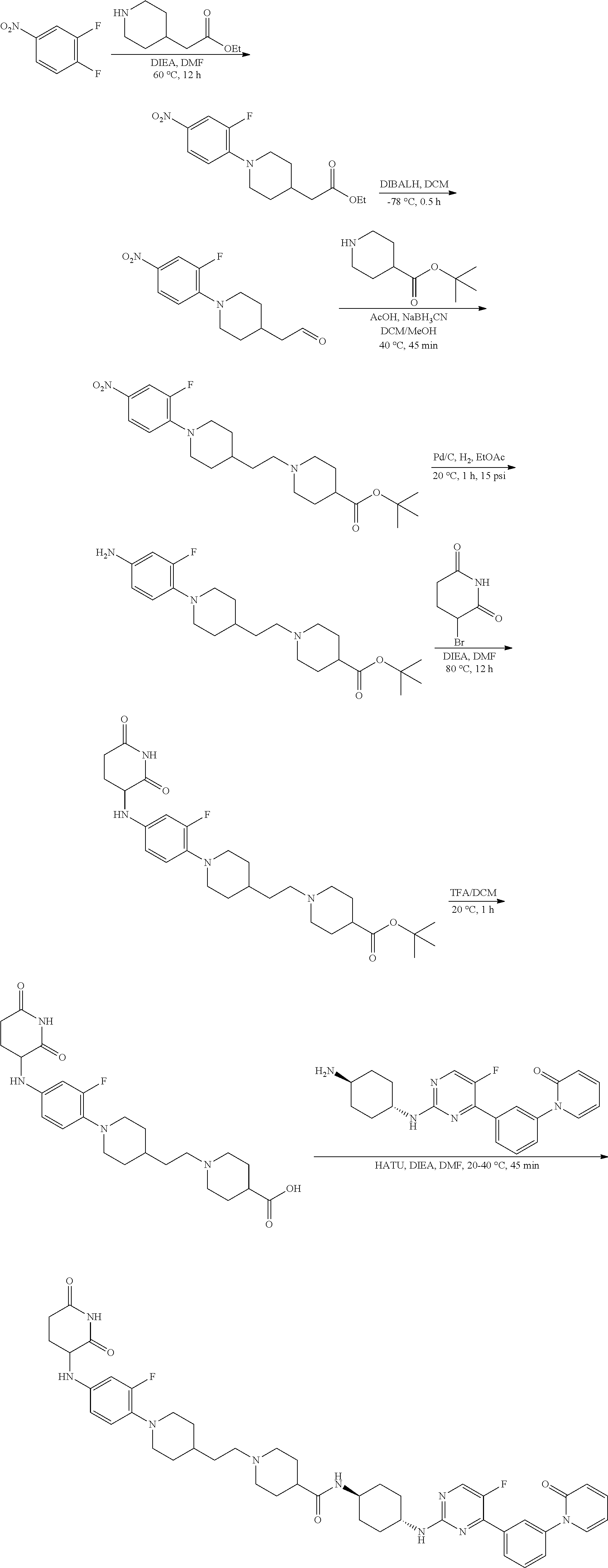

Synthesis of ethyl 2-[1-(2-fluoro-4-nitro-phenyl)-4-piperidyl]acetate. To a solution of 1,2-difluoro-4-nitro-benzene (3.87 g, 24.3 mmol, 2.69 mL, 1.00 eq) and ethyl 2-(4-piperidyl)acetate (4.16 g, 24.3 mmol, 1.00 eq) in DMF (20 mL) was added DIEA (4.71 g, 36.5 mmol, 6.35 mL, 1.50 eq) at 20 °C, the mixture was stirred at 60 °C for 12 h. LCMS showed the desired mass was detected. The mixture was added to H₂O (50 mL) and filtered. The filter cake was dried under reduced pressure to give the title compound (6.00 g, crude) as a yellow solid. LCMS (ES⁺): m/z 311.1 [M+H]⁺.

Synthesis of 2-(1-(2-fluoro-4-nitrophenyl)piperidin-4-yl)acetaldehyde. To a solution of ethyl 2-[1-(2-fluoro-4-nitro-phenyl)-4-piperidyl]acetate (3.00 g, 9.67 mmol, 1.00 eq) in DCM (40 mL) was added DIBAL-H (9.67 mL, 1.00 eq, 1 M in Tol.) at −78 °C under N₂, the mixture was stirred at −78 °C for 0.5 h under N₂ atmosphere. LCMS showed that ethyl 2-[1-(2-fluoro-4-nitro-phenyl)-4-piperidyl]acetate was consumed completely and the desired mass was detected. The mixture was quenched by the addition of methanol (40 mL) dropwise at −30 °C and stirred at −30 °C for 0.5 h. Then the mixture was poured into saturated aqueous sodium potassium tartrate solution (100 mL), the resulting mixture was stirred vigorously at 20 °C for 1 h and extracted with DCM (50 mL*3). The combined organic layers were dried over Na₂SO₄, filtered and the filtrate was concentrated under reduced pressure to give the title compound (2.50 g, crude) as a yellow solid. LCMS (ES⁺): m/z 267.1 [M+H]⁺.

Synthesis of tert-butyl 1-[2-[1-(2-fluoro-4-nitro-phenyl)-4-piperidyl]ethyl]piperidine-4-carboxylate. To a solution of 2-(1-(2-fluoro-4-nitrophenyl)piperidin-4-yl)acetaldehyde (2.00 g, 7.51 mmol, 1.00 eq) and tert-butyl piperidine-4-carboxylate (1.39 g, 7.51 mmol, 1.00 eq) in DCM (10 mL) and MeOH (10 mL) was added AcOH (451 mg, 7.51 mmol, 430 μL, 1.00 eq) at 20 °C, the mixture was stirred at 40 °C for 0.5 h. Then NaBH₃CN (944 mg, 15.0 mmol, 2.00 eq) was added at 40 °C, the mixture was stirred at 40 °C for 15 min. LCMS showed that 2-(1-(2-fluoro-4-nitrophenyl)piperidin-4-yl)acetaldehyde was consumed completely and the desired mass was detected. To the mixture was added H₂O (30 mL) and the mixture was extracted with EtOAc (20 mL*3). The combined organic layers were washed with brine (20 mL*3), dried over Na₂SO₄, filtered and the filtrate was concentrated under reduced pressure to give the title compound (3.50 g, crude) as a yellow solid. LCMS (ES⁺): m/z 436.2 [M+H]⁺.

Synthesis of tert-butyl 1-[2-[1-(4-amino-2-fluoro-phenyl)-4-piperidyl]ethyl]piperidine-4-carboxylate. To a solution of tert-butyl 1-[2-[1-(2-fluoro-4-nitro-phenyl)-4-piperidyl]ethyl]piperidine-4-carboxylate (3.20 g, 7.35 mmol, 1.00 eq) in EtOAc (30 mL) was added 10% Pd/C (4.00 g, 50% purity) at 20 °C under N₂, the mixture was degassed and purged with H₂ for three times, then the mixture was stirred at 20 °C for 1 h under H₂ (15 psi). LCMS showed that tert-butyl 1-[2-[1-(2-fluoro-4-nitro-phenyl)-4-piperidyl]ethyl]piperidine-4-carboxylate was consumed completely and the desired mass was detected. The mixture was filtered and the filtrate was concentrated under reduced pressure to give the title compound (2.30 g, crude) as a yellow solid. LCMS (ES⁺): m/z 406.3 [M+H]⁺.

Synthesis of tert-butyl 1-[2-[1-[4-[(2,6-dioxo-3-piperidyl)amino]-2-fluoro-phenyl]-4-piperidyl]ethyl]piperidine-4-carboxylate. To a solution of tert-butyl 1-[2-[1-(4-amino-2-fluoro-phenyl)-4-piperidyl]ethyl]piperidine-4-carboxylate (2.30 g, 5.67 mmol, 1.00 eq) and 3-bromopiperidine-2,6-dione (4.36 g, 22.7 mmol, 4.00 eq) in DMF (30 mL) was added DIEA (2.93 g, 22.7 mmol, 3.95 mL, 4.00 eq) at 20 °C, the mixture was stirred at 80 °C for 12 h. LCMS showed that tert-butyl 1-[2-[1-(4-amino-2-fluoro-phenyl)-4-piperidyl]ethyl]piperidine-4-carboxylate was consumed completely and the desired mass was detected. To the mixture was added H₂O (30 mL) and the mixture was extracted with EtOAc (20 mL*3). The combined organic layers were washed with brine (20 mL*3), dried over Na₂SO₄, filtered and the filtrate was concentrated under reduced pressure to give a residue. The residue was purified by MPLC (SiO₂, Petroleum ether/Ethyl acetate=1/0 to 3/1) to give the title compound (1.50 g, 2.90 mmol, 51.2% yield) as a yellow solid. LCMS (ES⁺): m/z 517.3 [M+H]⁺.

Synthesis of 1-[2-[1-[4-[(2,6-dioxo-3-piperidyl)amino]-2-fluoro-phenyl]-4-piperidyl]ethyl]piperidine-4-carboxylic acid. To a solution of tert-butyl 1-[2-[1-[4-[(2,6-dioxo-3-piperidyl)amino]-2-fluoro-phenyl]-4-piperidyl]ethyl]piperidine-4-carboxylate (100 mg, 194 μmol, 1.00 eq) in DCM (4 mL) was added TFA (110 mg, 968 μmol, 71.9 μL, 5.00 eq) at 20 °C, the mixture was stirred at 20 °C for 1 h. LCMS showed that tert-butyl 1-[2-[1-[4-[(2,6-dioxo-3-piperidyl)amino]-2-fluoro-phenyl]-4-piperidyl]ethyl]piperidine-4-carboxylate was consumed completely and the desired mass was detected. The mixture was concentrated under reduced pressure to give the title compound (89.0 mg, crude) as a yellow oil liquid. LCMS (ES⁺): m/z 461.3 [M+H]⁺.

Synthesis of trans-1-[2-[1-[4-[(2,6-dioxo-3-piperidyl)amino]-2-fluoro-phenyl]-4-piperidyl]ethyl]-N-[4-[[5-fluoro-4-[3-(2-oxo-1-pyridyl)phenyl]pyrimidin-2-yl]amino]cyclohexyl]piperidine-4-carboxamide (Compound 316). To a solution of 1-[2-[1-[4-[(2,6-dioxo-3-piperidyl)amino]-2-fluoro-phenyl]-4-piperidyl]ethyl]piperidine-4-carboxylic acid (89.0 mg, 193 μmol, 1.00 eq) and HATU (147 mg, 387 μmol, 2.00 eq) in DMF (2 mL) was added DIEA (125 mg, 968 μmol, 168 μL, 5.00 eq) at 20 °C, the mixture was stirred at 40 °C for 15 min. trans-1-[3-[2-[(4-aminocyclohexyl)amino]-5-fluoro-pyrimidin-4-yl]phenyl]pyridin-2-one (95.4 mg, 193 μmol, 1.00 eq, TFA) was added at 40 °C, the mixture was stirred at 40 °C for 0.5 h. LCMS showed that 1-[2-[1-[4-[(2,6-dioxo-3-piperidyl)amino]-2-fluoro-phenyl]-4-piperidyl]ethyl]piperidine-4-carboxylic acid was consumed completely and the desired mass was detected. To the mixture was added H₂O (3 mL) and the mixture was extracted with EtOAc (3 mL*3). The combined organic layers were washed with brine (3 mL*3), dried over Na₂SO₄, filtered and the filtrate was concentrated under reduced pressure to give a residue. The residue was purified by prep-HPLC (column: Phenomenex Luna C18 80*30 mm*3 um; mobile phase: [H₂O (0.1% TFA)-ACN]; gradient: 15%-45% B over 8.0 min) to give the title compound (Compound 316) (22.7 mg, 26.0 μmol, 13.5% yield, 94.3% purity) as a white solid. LCMS (ES⁺): m/z 822.4 [M+H]⁺

### Example 350. Synthesis of trans-2-(4-((4-(3-(2,6-dioxopiperidin-3-yl)-2-oxo-2,3-dihydrobenzo[d]oxazol-6-yl)piperazin-1-yl)methyl)piperidin-1-yl)-N-(4-((5-fluoro-4-(3-(2-oxopyridin-1(2H)-yl)phenyl)pyrimidin-2-yl)amino)cyclohexyl)acetamide (Compound 339)

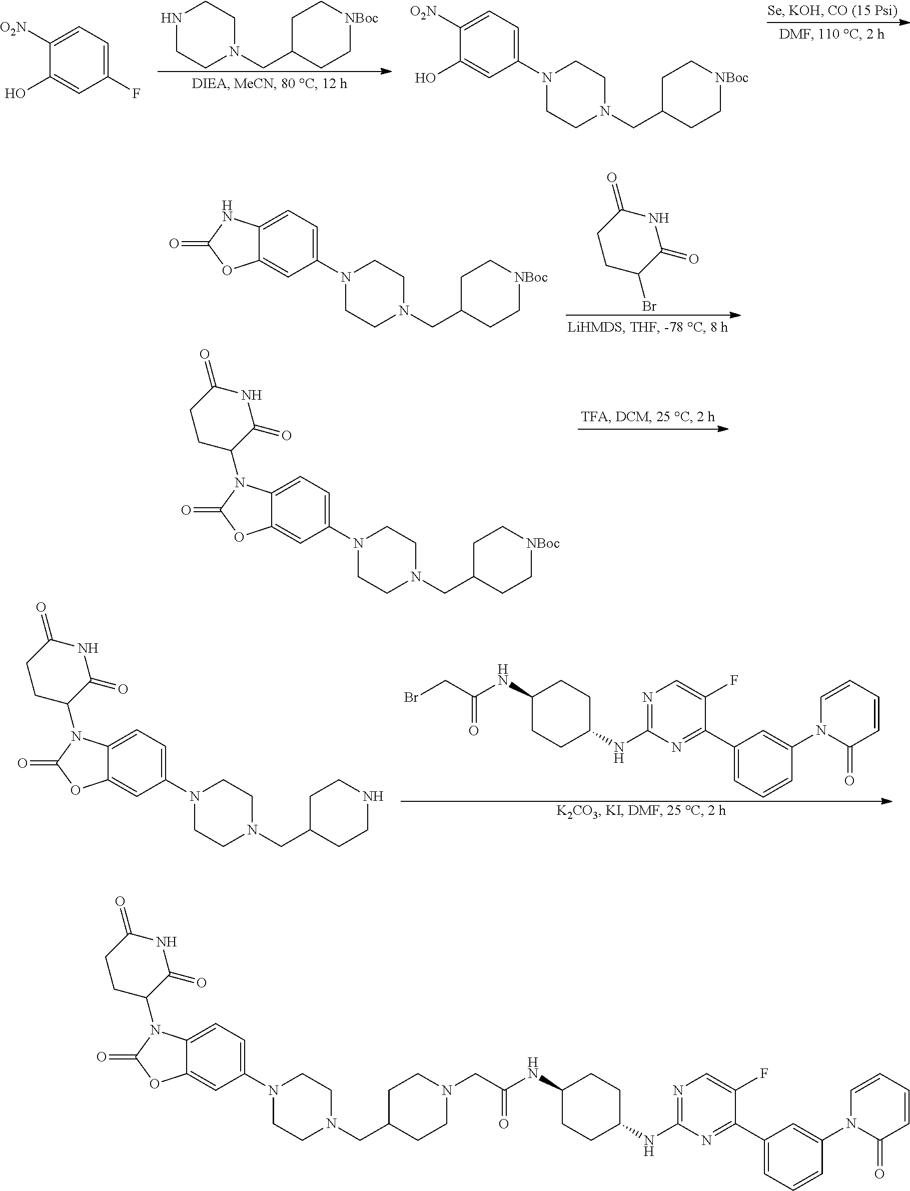

Synthesis of tert-butyl 4-((4-(3-hydroxy-4-nitrophenyl)piperazin-1-yl)methyl)piperidine-1-carboxylate. To a solution of tert-butyl 4-(piperazin-1-ylmethyl)piperidine-1-carboxylate (10.0 g, 35.2 mmol, 1.00 eq) in MeCN (70.0 mL) was added DIEA (9.12 g, 70.5 mmol, 12.2 mL, 2.00 eq) and 5-fluoro-2-nitro-phenol (6.65 g, 42.3 mmol, 1.20 eq) at 25 °C. The mixture was stirred at 80 °C for 12 h. LC-MS showed tert-butyl 4-(piperazin-1-ylmethyl)piperidine-1-carboxylate was consumed completely and the desired mass was detected. The reaction mixture was filtered and concentrated under reduced pressure. The crude product was triturated with MeCN at 25 °C for 30 min to give the title compound (13.5 g, 26.4 mmol, 74.9% yield, 82.4% purity) as a white solid. LCMS (ES⁺): m/z 421.3 [M+H]⁺.

Synthesis of tert-butyl 4-((4-(2-oxo-2,3-dihydrobenzo[d]oxazol-6-yl)piperazin-1-yl)methyl)piperidine-1-carboxylate. To a solution of tert-butyl 4-((4-(3-hydroxy-4-nitrophenyl)piperazin-1-yl)methyl)piperidine-1-carboxylate (3.00 g, 7.13 mmol, 1.00 eq) in DMF (30.0 mL) was added KOH (800 mg, 14.2 mmol, 2.00 eq) and Se (563 mg, 7.13 mmol, 1.00 eq) at 25 °C under N₂ atmosphere. The suspension was degassed and purged with CO for 3 times. The mixture was stirred under CO (7.13 mmol, 1.00 eq) (15 Psi) at 110 °C for 12 h. LC-MS showed tert-butyl 4-((4-(3-hydroxy-4-nitrophenyl)piperazin-1-yl)methyl)piperidine-1-carboxylate was consumed completely and the desired mass was detected. The reaction diluted with H₂O 60 mL and combined organic layers were washed with EtOAc (100 mL*3) and concentrated under reduced pressure. The residue was purified by column chromatography (SiO₂, Dichloromethane:Methanol=1:0 to 5:1) to give the title compound (8.00 g, 14.4 mmol, 50.7% yield, 75.4% purity) as a yellow solid. LCMS (ES⁺): m/z 417.3 [M+H]⁺.

Synthesis of tert-butyl 4-((4-(3-(2,6-dioxopiperidin-3-yl)-2-oxo-2,3-dihydrobenzo[d]oxazol-6-yl)piperazin-1-yl)methyl)piperidine-1-carboxylate. The reaction was set up in two parallel batches. To a solution of tert-butyl 4-[[4-(2-oxo-3H-1,3-benzoxazol-6-yl)piperazin-1-yl]methyl]piperidine-1-carboxylate (3.00 g, 7.20 mmol, 1.00 eq) in THE (20.0 mL) was added LiHMDS (1.00 M, 14.4 mL, 2.00 eq) at −78 °C in N₂ for 1 h, and add 3-bromopiperidine-2,6-dione (4.15 g, 21.6 mmol, 3.00 eq) at −78 °C. The mixture was stirred at −78 °C for 7 h. One batches in parallel were monitored in random. LC-MS showed tert-butyl 4-[[4-(2-oxo-3H-1,3-benzoxazol-6-yl)piperazin-1-yl]methyl]piperidine-1-carboxylate was consumed completely and the desired mass was detected. The two batches were combined. The reaction mixture was quenched by addition H₂O 10 mL at 0 °C, and extracted with EtOAc (30 mL*3). The combined dried over Na₂SO₄, filtered and concentrated under reduced pressure. The residue was purified by column chromatography (SiO₂, Petroleum ether:THF=1:0 to 0:1) to give the title compound (6.00 g, 9.83 mmol, 68.2% yield, 86.4% purity) as a green oil. LCMS (ES⁺): m/z 528.4 [M+H]⁺.

Synthesis of 3-(2-oxo-6-(4-(piperidin-4-ylmethyl)piperazin-1-yl)benzo[d]oxazol-3 (2H)-yl)piperidine-2,6-dione. To a solution of tert-butyl 4-((4-(3-(2,6-dioxopiperidin-3-yl)-2-oxo-2,3-dihydrobenzo[d]oxazol-6-yl)piperazin-1-yl)methyl)piperidine-1-carboxylate (1.00 g, 1.90 mmol, 1.00 eq) in DCM (3.00 mL) was added TFA (1.54 g, 13.4 mmol, 1.00 mL, 7.10 eq) at 25 °C. The mixture was stirred at 25 °C for 2 h. LC-MS showed tert-butyl 4-((4-(3-(2,6-dioxopiperidin-3-yl)-2-oxo-2,3-dihydrobenzo[d]oxazol-6-yl)piperazin-1-yl)methyl)piperidine-1-carboxylate was consumed completely and the desired mass was detected. The reaction mixture was concentrated under reduced pressure. The residue was purified by prep-HPLC (column: Phenomenex luna C18 250*50 mm*10 um; mobile phase: [H₂O (0.1% TFA)-ACN]; gradient: 1%-30% B over 10.0 min) to give the title compound (100 mg, 180.8 μmol, 9.54% yield, 97.9% purity) as a white solid. LCMS (ES⁺): m/z 428.3 [M+H]⁺.

Synthesis of trans-2-(4-((4-(3-(2,6-dioxopiperidin-3-yl)-2-oxo-2,3-dihydrobenzo[d]oxazol-6-yl)piperazin-1-yl)methyl)piperidin-1-yl)-N-(4-((5-fluoro-4-(3-(2-oxopyridin-1(2H)-yl)phenyl)pyrimidin-2-yl)amino)cyclohexyl)acetamide (Compound 339). To a solution of 3-(2-oxo-6-(4-(piperidin-4-ylmethyl)piperazin-1-yl)benzo[d]oxazol-3 (2H)-yl)piperidine-2,6-dione (100 mg, 233 μmol, 1.00 eq) in DMF (2.00 mL) was added K₂CO₃ (64.6 mg, 467 μmol, 2.00 eq), trans-2-bromo-N-(4-((5-fluoro-4-(3-(2-oxopyridin-1(2H)-yl)phenyl)pyrimidin-2-yl)amino)cyclohexyl)acetamide (117 mg, 233 μmol, 1.00 eq) and KI (38.8 mg, 233 μmol, 1.00 eq) at 25 °C. The mixture was stirred at 25 °C for 2 h. LC-MS showed 3-(2-oxo-6-(4-(piperidin-4-ylmethyl)piperazin-1-yl)benzo[d]oxazol-3 (2H)-yl)piperidine-2,6-dione was consumed completely and the desired mass was detected. The reaction mixture was concentrated under reduced pressure. The residue was purified by prep-HPLC (column: Phenomenex Luna C18 75*30 mm*3 um; mobile phase: [H₂O (0.1% TFA)-ACN]; gradient: 5%-35% B over 8.0 min) to give the title compound (Compound 339) (69.8 mg, 72.2 μmol, 30.9% yield, 99.5% purity, TFA) as a yellow solid. LCMS (ES⁺): m/z 847.5 [M+H]⁺.

### Example 351. Synthesis of trans-2-(4-((4-(4-((2,6-dioxopiperidin-3-yl)amino)-2-fluorophenyl)piperazin-1-yl)methyl)piperidin-1-yl)-N-(4-((5-fluoro-4-(3-(2-oxopyridin-1(2H)-yl)phenyl)pyrimidin-2-yl)amino)cyclohexyl)acetamide (Compound 326)

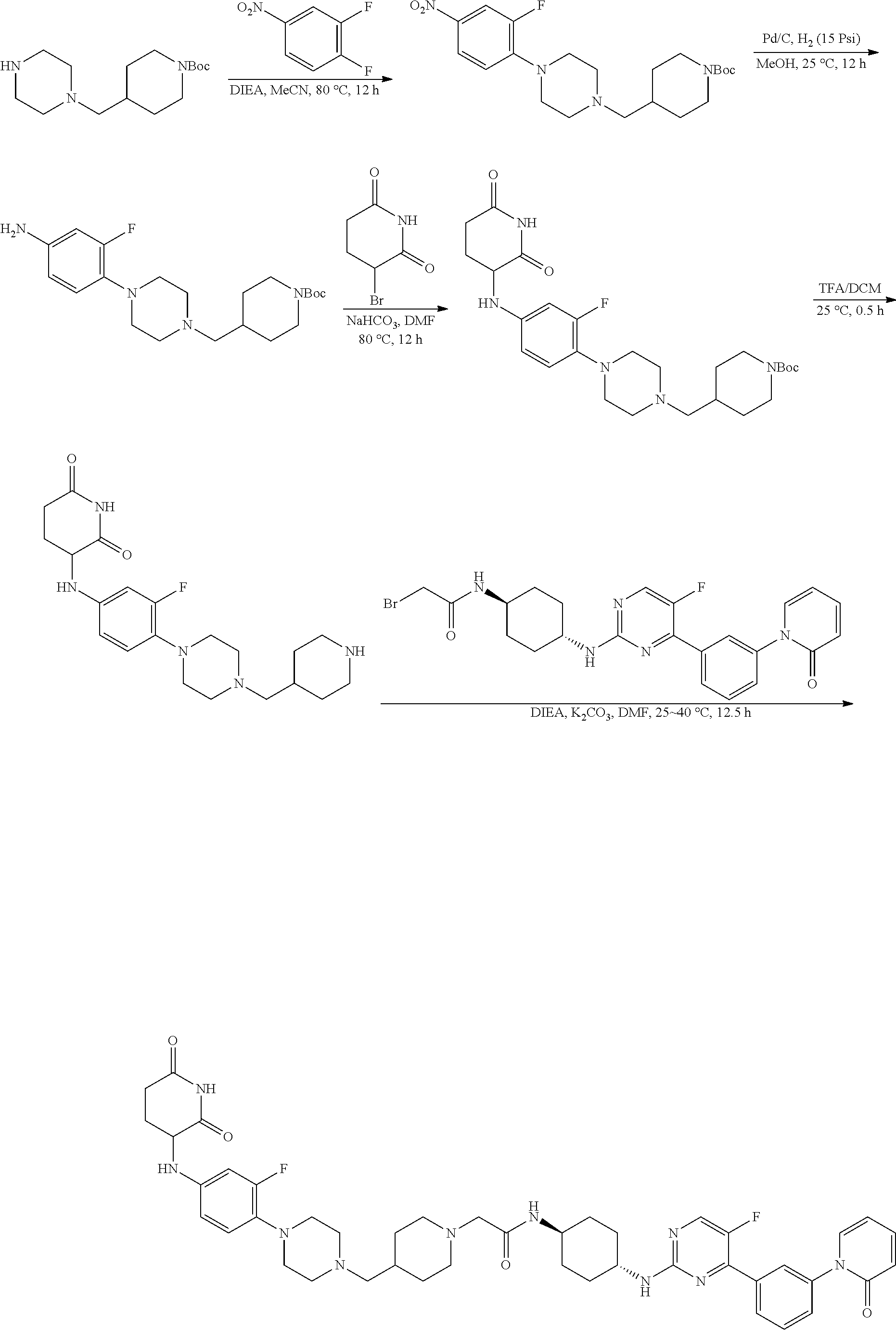

Synthesis of tert-butyl 4-((4-(2-fluoro-4-nitrophenyl)piperazin-1-yl)methyl)piperidine-1-carboxylate. To a solution of tert-butyl 4-(piperazin-1-ylmethyl)piperidine-1-carboxylate (7.00 g, 24.7 mmol, 1.00 eq) in MeCN (100 mL) was added DIEA (9.58 g, 74.1 mmol, 12.9 mL, 3.00 eq) and 1,2-difluoro-4-nitro-benzene (3.93 g, 24.7 mmol, 2.73 mL, 1.00 eq) at 25 °C. The mixture was stirred at 80 °C for 2 h. LC-MS showed tert-butyl 4-(piperazin-1-ylmethyl)piperidine-1-carboxylate was consumed completely and the desired mass was detected. The reaction mixture was filtered to get crude product. The crude product was purified by re-crystallization from MeCN (30 mL) at 20 °C to give the title compound (10.0 g, 22.4 mmol, 91.0% yield, 95.0% purity) as a yellow solid. LCMS (ES⁺): m/z 423.2 [M+H]⁺.

Synthesis of tert-butyl 4-((4-(4-amino-2-fluorophenyl)piperazin-1-yl)methyl)piperidine-1-carboxylate. To a solution of tert-butyl 4-((4-(2-fluoro-4-nitrophenyl)piperazin-1-yl)methyl)piperidine-1-carboxylate (5.00 g, 11.8 mmol, 1.00 eq) in MeOH (3 mL) was added 10% Pd/C (800 mg, 3.76 mmol, 50.0% purity, 3.18 eq) under N₂ at 25 °C. The suspension was degassed under vacuum and purged with H₂ several times. The mixture was stirred under H₂ (15 Psi) at 25 °C for 12 h. LC-MS showed tert-butyl 4-((4-(2-fluoro-4-nitrophenyl)piperazin-1-yl)methyl)piperidine-1-carboxylate was consumed completely and the desired mass was detected. The reaction mixture was filtered and the filter was concentrated to give the title compound (4.70 g, crude) as a brown solid. LCMS (ES⁺): m/z 393.3.1 [M+H]⁺.

Synthesis of tert-butyl 4-((4-(4-((2,6-dioxopiperidin-3-yl)amino)-2-fluorophenyl)piperazin-1-yl)methyl)piperidine-1-carboxylate. To a solution of tert-butyl 4-((4-(4-amino-2-fluorophenyl)piperazin-1-yl)methyl)piperidine-1-carboxylate (4.60 g, 11.7 mmol, 1.00 eq) in DMF (40 mL) was added NaHCO₃ (2.95 g, 35.1 mmol, 1.37 mL, 3.00 eq) and 3-bromopiperidine-2,6-dione (4.50 g, 23.4 mmol, 2.00 eq) at 25 °C. The mixture was stirred at 80 °C for 12 h. LC-MS showed tert-butyl 4-((4-(4-amino-2-fluorophenyl)piperazin-1-yl)methyl)piperidine-1-carboxylate was consumed completely and the desired mass was detected. The reaction mixture was diluted with H₂O 50 mL and extracted with EtOAc (50 mL*3). The combined organic layers were concentrated under reduced pressure. The residue was purified by column chromatography (SiO₂, Petroleum ether:THF=1:0 to 0:1) to give the title compound (5.10 g, 9.59 mmol, 81.8% yield, 94.7% purity) as a purple solid. LCMS (ES⁺): m/z 504.3 [M+H]⁺.

Synthesis of 3-((3-fluoro-4-(4-(piperidin-4-ylmethyl)piperazin-1-yl)phenyl)amino)piperidine-2,6-dione. To a solution of tert-butyl 4-((4-(4-((2,6-dioxopiperidin-3-yl)amino)-2-fluorophenyl)piperazin-1-yl)methyl)piperidine-1-carboxylate (2.00 g, 3.97 mmol, 1.00 eq) in DCM (20 mL) was added TFA (7.68 g, 67.3 mmol, 5.00 mL, 16.9 eq) at 25 °C. The mixture was stirred at 25 °C for 0.5 h. LC-MS showed tert-butyl 4-((4-(4-((2,6-dioxopiperidin-3-yl)amino)-2-fluorophenyl)piperazin-1-yl)methyl)piperidine-1-carboxylate was consumed completely and the desired mass was detected. The reaction was concentrated under reduced pressure to give the title compound (2.00 g, crude, TFA) as a black oil. LCMS (ES⁺): m/z 404.3 [M+H]⁺.

Synthesis of trans-2-(4-((4-(4-((2,6-dioxopiperidin-3-yl)amino)-2-fluorophenyl)piperazin-1-yl)methyl)piperidin-1-yl)-N-(4-((5-fluoro-4-(3-(2-oxopyridin-1(2H)-yl)phenyl)pyrimidin-2-yl)amino)cyclohexyl)acetamide (Compound 326). To a solution of 3-((3-fluoro-4-(4-(piperidin-4-ylmethyl)piperazin-1-yl)phenyl)amino)piperidine-2,6-dione (200 mg, 386 μmol, 1.00 eq, TFA) in DMF (5 mL) was added and DIEA (49.9 mg, 386 μmol, 67.3 μL, 1.00 eq) at 25 °C. The mixture was stirred at 25 °C for 0.5 h. Then 2-bromo-N-[4-[[5-fluoro-4-[3-(2-oxo-1-pyridyl)phenyl]pyrimidin-2-yl]amino]cyclohexyl]acetamide (96.6 mg, 193 μmol, 0.500 eq) and K₂CO₃ (106 mg, 772 μmol, 2.00 eq) was added at 25 °C. The mixture was stirred at 40 °C for 12 h. LC-MS showed 3-((3-fluoro-4-(4-(piperidin-4-ylmethyl)piperazin-1-yl)phenyl)amino)piperidine-2,6-dione was consumed completely and the desired mass was detected. The reaction mixture was diluted with H₂O 10 mL and extracted with EtOAc (20 mL*3). The combined organic layers were concentrated under reduced pressure. The residue was purified by prep-HPLC (column: Waters Xbridge BEH C18 100*30 mm*10 um; mobile phase: [H₂O (10 mM NH₄HCO₃)-ACN]; gradient: 25%-55% B over 8.0 min) to give the title compound (Compound 326) (18.4 mg, 20.8 μmol, 5.40% yield, 93.2% purity) as a white solid. LCMS (ES⁺): m/z 823.4 [M+H]⁺.

### Example 352. Synthesis of trans-2-(1-((1-(3-(2,6-dioxopiperidin-3-yl)-2-oxo-2,3-dihydrobenzo[d]oxazol-6-yl)piperidin-4-yl)methyl)piperidin-4-yl)-N-(4-((5-fluoro-4-(3-(2-oxopyridin-1(2H)-yl)phenyl)pyrimidin-2-yl)amino)cyclohexyl)acetamide (Compound 358)

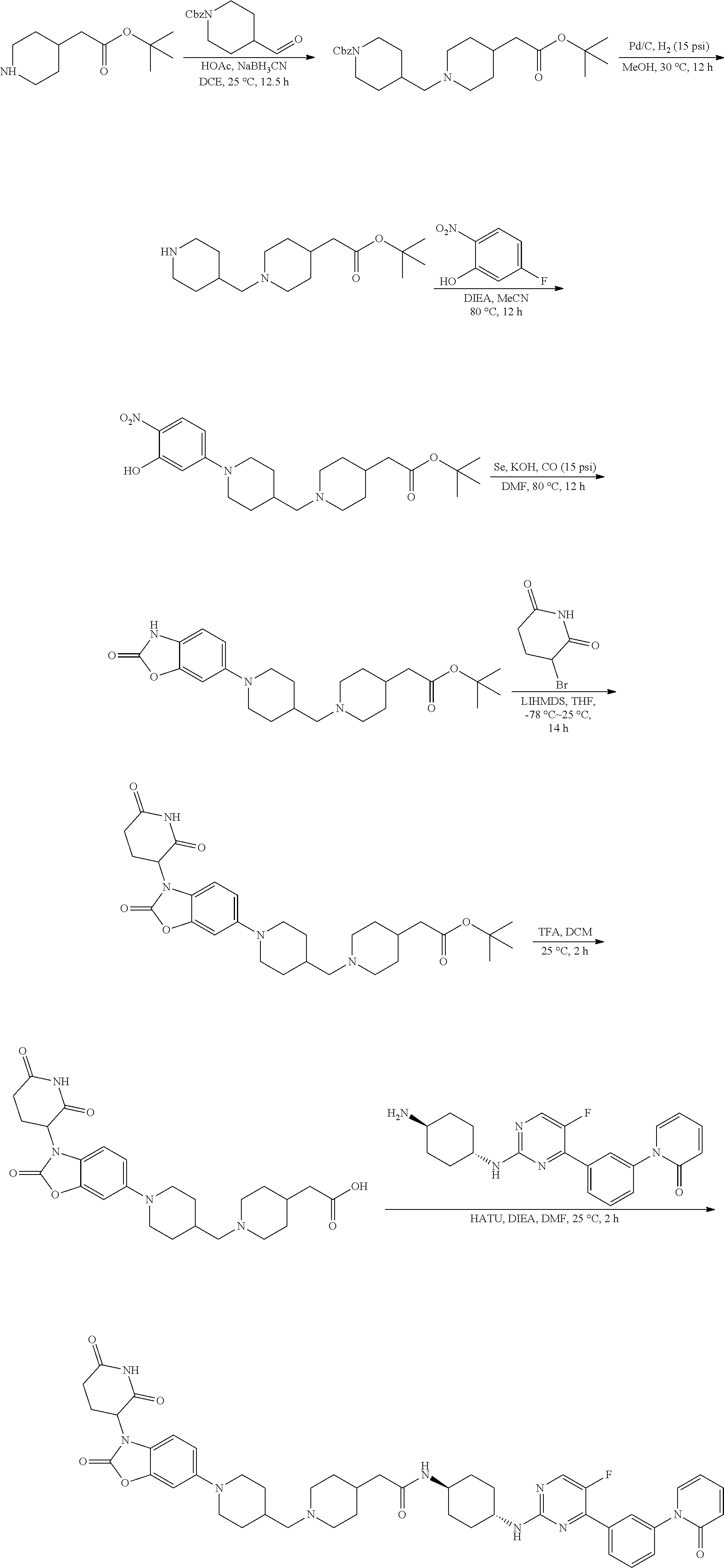

Synthesis of benzyl 4-((4-(2-(tert-butoxy)-2-oxoethyl)piperidin-1-yl)methyl)piperidine-1-carboxylate. To a solution of tert-butyl 2-(piperidin-4-yl)acetate (5.00 g, 25.0 mmol, 1.00 eq) in DCE (100 mL) was added HOAc (7.53 g, 125 mmol, 7.18 mL, 5.00 eq) and benzyl 4-formylpiperidine-1-carboxylate (15.1 g, 62.7 mmol, 2.50 eq) at 25 °C, The mixture was stirred at 25 °C for 0.5 h. Then NaBH₃CN (7.88 g, 125 mmol, 5.00 eq) was added at 25 °C. The mixture was stirred at 25 °C for 12 h. LC-MS showed desired mass was detected. The reaction mixture was diluted with water 80 mL and extracted with DCM (100 mL*3), filtered and concentrated under reduced pressure. The residue was purified by column chromatography (SiO₂, DCM:MeOH=1:0 to 5:1) to give the title compound (7.00 g, 15.2 mmol, 60.9% yield, 94.0% purity) as a white oil. LCMS (ES⁺): m/z 431.2 [M+H]⁺.

Synthesis of tert-butyl 2-(1-(piperidin-4-ylmethyl)piperidin-4-yl)acetate. To a solution of 10% Pd/C (3.50 g, 16.4 mmol, 50.0% purity, 2.02 eq) in MeOH (40.0 mL) was added benzyl 4-((4-(2-(tert-butoxy)-2-oxoethyl)piperidin-1-yl)methyl)piperidine-1-carboxylate (3.50 g, 8.13 mmol, 1.00 eq) at 25 °C under N₂ atmosphere. The suspension was degassed and purged with H₂ for 3 times. The mixture was stirred under H₂ (15 Psi) at 30 °C for 12 h. LC-MS showed desired mass was detected. The reaction mixture was filtered and the filtrate concentrated under high vacuum to give the title compound (4.17 g, crude) as a white oil. LCMS (ES⁺): m/z 297.2 [M+H]⁺.

Synthesis of tert-butyl 2-[1-[[1-(3-hydroxy-4-nitro-phenyl)-4-piperidyl]methyl]-4-piperidyl]acetate. To a solution of tert-butyl 2-[1-(4-piperidylmethyl)-4-piperidyl]acetate (5.00 g, 16.9 mmol, 1.00 eq) in MeCN (50 mL) was added DIEA (6.54 g, 50.6 mmol, 8.81 mL, 3.00 eq) and 5-fluoro-2-nitro-phenol (2.65 g, 16.9 mmol, 1.00 eq) at 25 °C. The mixture was stirred at 80 °C for 12 h. LC-MS showed the desired mass was detected. The reaction mixture was concentrated under reduced pressure. The crude product was purified by re-crystallization from Petroleum ether (30 mL) at 25 °C to give the title compound (9.12 g, crude) as a yellow solid. LCMS (ES⁺): m/z 434.2 [M+H]⁺.

Synthesis of tert-butyl 2-[1-[[1-(2-oxo-3H-1, 3-benzoxazol-6-yl)-4-piperidyl]methyl]-4-piperidyl]acetate. A mixture of tert-butyl 2-[1-[[1-(3-hydroxy-4-nitro-phenyl)-4-piperidyl]methyl]-4-piperidyl]acetate (4.50 g, 10.4 mmol, 1.00 eq), Se (840 mg, 10.4 mmol, 1.00 eq) and KOH (1.16 g, 20.8 mmol, 2.00 eq) in DMF (10 mL) at 25 °C was degassed and purged with CO (10.4 mmol, 1.00 eq) for 3 times, and then the mixture was stirred at 80 °C for 12 h under CO (10.4 mmol, 1.00 eq) atmosphere. LC-MS showed the desired mass was detected. The reaction mixture was diluted with water 20 mL and extracted with EtOAc (40 mL*3), filtered and concentrated under reduced pressure. The residue was purified by column chromatography (SiO₂, Dichloromethane:Methanol=1:0 to 5:1) to give the title compound (3.34 g, 5.04 mmol, 24.3% yield, 64.8% purity) as a yellow solid. LCMS (ES⁺): m/z 430.3 [M+H]⁺.

Synthesis of tert-butyl 2-(1-((1-(3-(2,6-dioxopiperidin-3-yl)-2-oxo-2,3-dihydrobenzo[d]oxazol-6-yl)piperidin-4-yl)methyl)piperidin-4-yl)acetate. To a solution of tert-butyl 2-[1-[[1-(2-oxo-3H-1,3-benzoxazol-6-yl)-4-piperidyl]methyl]-4-piperidyl]acetate (1.30 g, 3.03 mmol, 1.00 eq) in THF (20 mL) was added LiHMDS (1 M, 12.1 mL, 4.00 eq) at −78 °C in N₂ for 1 h, and add 3-bromopiperidine-2,6-dione (3.49 g, 18.1 mmol, 6.00 eq) at −78 °C for 1 h. The mixture was stirred at 25 °C for 12 h. LC-MS showed the desired mass was detected. The two batches were combined. The reaction mixture was quenched by addition H₂O 10 mL at 0 °C, and extracted with EtOAc (20 mL*3), filtered and concentrated under reduced pressure. The residue was purified by column chromatography (SiO₂, PE:THF=1:0 to 1:1) to give the title compound (80.0 mg, 131 μmol, 4.36% yield, 89.2% purity) as a yellow oil. LCMS (ES⁺): m/z 541.3 [M+H]⁺.

Synthesis of 2-(1-((1-(3-(2,6-dioxopiperidin-3-yl)-2-oxo-2,3-dihydrobenzo[d]oxazol-6-yl)piperidin-4-yl)methyl)piperidin-4-yl)acetic acid. To a solution of tert-butyl 2-(1-((1-(3-(2,6-dioxopiperidin-3-yl)-2-oxo-2,3-dihydrobenzo[d]oxazol-6-yl)piperidin-4-yl)methyl)piperidin-4-yl)acetate (80.0 mg, 147 μmol, 1.00 eq in DCM (1 mL) was added TFA (1.54 g, 13.5 mmol, 1 mL, 91.0 eq) at 25 °C. The mixture was stirred at 25 °C for 2 h. LC-MS showed the desired mass was detected. The reaction mixture was concentrated under reduced pressure to give a residue to give the title compound (200 mg, crude, TFA) as a yellow oil. LCMS (ES⁺): m/z 485.2 [M+H]⁺.

Synthesis of trans-2-(1-((1-(3-(2,6-dioxopiperidin-3-yl)-2-oxo-2,3-dihydrobenzo[d]oxazol-6-yl)piperidin-4-yl)methyl)piperidin-4-yl)-N-(4-((5-fluoro-4-(3-(2-oxopyridin-1(2H)-yl)phenyl)pyrimidin-2-yl)amino)cyclohexyl)acetamide (Compound 358). To a solution of 2-(1-((1-(3-(2,6-dioxopiperidin-3-yl)-2-oxo-2,3-dihydrobenzo[d]oxazol-6-yl)piperidin-4-yl)methyl)piperidin-4-yl)acetic acid (30.0 mg, 50.1 μmol, 1.00 eq, TFA) in DMF (1 ml) was added trans-1-(3-(2-((4-aminocyclohexyl)amino)-5-fluoropyrimidin-4-yl)phenyl)pyridin-2(1H)-one (19.0 mg, 45.7 μmol, 1.00 eq, HCl), HATU (28.6 mg, 75.2 μmol, 1.50 eq) and DIEA (19.4 mg, 150 μmol, 26.2 μL, 3.00 eq) at 25 °C. The mixture was stirred at 25 °C for 2 h. LC-MS showed the desired mass was detected. The reaction mixture was filtered to give a residue. The residue was purified by prep-HPLC (column: 3_Phenomenex Luna C18 75*30 mm*3 um; mobile phase: [H₂O (0.1% TFA)-ACN]; gradient: 5%-35% B over 8.0 min). The residue was purified by prep-HPLC (column: Waters Xbridge BEH C18 100*25 mm*10 um; mobile phase: [H₂O (10 mM NH₄HCO₃)-ACN]; gradient: 15%-55% B over 8.0 min) to give a residue to give the title compound (Compound 358) (10.3 mg, 12.2 μmol, 24.3% yield, 100% purity) as a white solid. LCMS (ES⁺): m/z 846.4 [M+H]⁺.

### Example 353. Synthesis of 2-(4-((1-(3-(2,6-dioxopiperidin-3-yl)-2-oxo-2,3-dihydrobenzo[d]oxazol-6-yl)piperidin-4-yl)methyl)piperazin-1-yl)-N-(trans-4-((5-fluoro-4-(3-(2-oxopyridin-1(2H)-yl)phenyl)pyrimidin-2-yl)amino)cyclohexyl)acetamide (Compound 360)

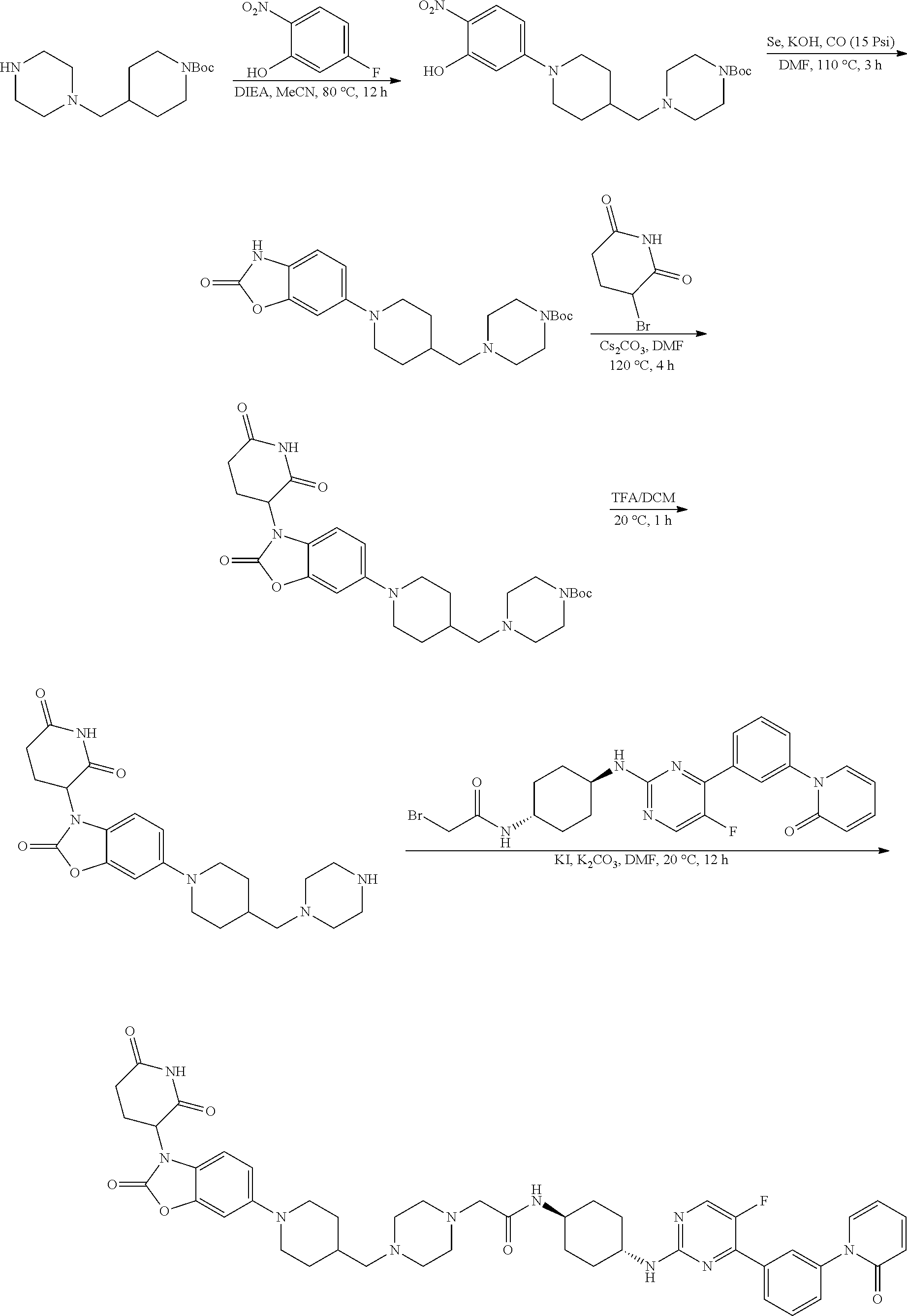

Synthesis of tert-butyl 4-((1-(3-hydroxy-4-nitrophenyl)piperidin-4-yl)methyl)piperazine-1-carboxylate. To a solution of tert-butyl 4-(4-piperidylmethyl)piperazine-1-carboxylate (5.00 g, 17.6 mmol, 1.00 eq) and 5-fluoro-2-nitro-phenol (3.33 g, 21.1 mmol, 1.20 eq) in MeCN (70 mL) was added DIEA (6.84 g, 52.9 mmol, 9.22 mL, 3.00 eq) at 20 °C. The mixture was stirred at 80 °C for 12 h. LC-MS showed the desired mass was detected. The reaction mixture was concentrated under reduced pressure. The crude product was triturated with petroleum ether (50 mL) at 20 °C for 30 min to give the title compound (11.0 g, crude) as a yellow solid. LCMS (ES⁺): m/z 421.2 [M+H]⁺.

Synthesis of tert-butyl 4-((1-(2-oxo-2,3-dihydrobenzo[d]oxazol-6-yl)piperidin-4-yl)methyl)piperazine-1-carboxylate. The reaction was set up in two batches. A mixture of tert-butyl 4-((1-(3-hydroxy-4-nitrophenyl)piperidin-4-yl)methyl)piperazine-1-carboxylate (5.00 g, 11.8 mmol, 1.00 eq), Se (938 mg, 11.8 mmol, 1.00 eq), KOH (1.33 g, 23.7 mmol, 2.00 eq) in DMF (30 mL) was degassed and purged with CO for 3 times at 20 °C, and then the mixture was stirred at 110 °C for 3 h under CO (15 Psi) atmosphere. Two batch in parallel was monitored in random. LC-MS showed the desired mass was detected. Two batches in parallel were combined. The reaction mixture was diluted with H₂O 100 mL and extracted with EtOAc 100 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (20.0 g, crude) as a purple oil. LCMS (ES⁺): m/z 417.3 [M+H]⁺.

Synthesis of tert-butyl 4-((1-(3-(2,6-dioxopiperidin-3-yl)-2-oxo-2,3-dihydrobenzo[d]oxazol-6-yl)piperidin-4-yl)methyl)piperazine-1-carboxylate. The reaction was set up in three batches. To a solution of tert-butyl 4-((1-(2-oxo-2,3-dihydrobenzo[d]oxazol-6-yl)piperidin-4-yl)methyl)piperazine-1-carboxylate (4.00 g, 9.60 mmol, 1.00 eq) and 3-bromopiperidine-2,6-dione (7.38 g, 38.4 mmol, 4.00 eq) in DMF (100 mL) was added Cs₂CO₃ (9.39 g, 28.8 mmol, 3.00 eq) at 20 °C. The mixture was stirred at 120 °C for 4 h. One batch in parallel was monitored in random. LC-MS showed the desired mass was detected. Three batches in parallel were combined. The reaction mixture was diluted with H₂O 30 mL and extracted with EtOAc 30 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (200 mg, 200 μmol, 6.97e-1% yield, 53% purity) as a purple solid. LCMS (ES⁺): m/z 528.2 [M+H]⁺.

Synthesis of 3-(2-oxo-6-(4-(piperazin-1-ylmethyl)piperidin-1-yl)benzo[d]oxazol-3 (2H)-yl)piperidine-2,6-dione. To a solution of tert-butyl 4-((1-(3-(2,6-dioxopiperidin-3-yl)-2-oxo-2,3-dihydrobenzo[d]oxazol-6-yl)piperidin-4-yl)methyl)piperazine-1-carboxylate (60.0 mg, 113 μmol, 1.00 eq) in DCM (1 mL) was added TFA (460 mg, 4.04 mmol, 0.300 mL, 35.5 eq) at 20 °C. The mixture was stirred at 20 °C for 1 h. LC-MS showed the desired mass was detected. The reaction mixture was concentrated under reduced pressure to give the title compound (70 mg, crude, TFA) as a purple oil. LCMS (ES⁺): m/z 428.2 [M+H]⁺

Synthesis of 2-(4-((1-(3-(2,6-dioxopiperidin-3-yl)-2-oxo-2,3-dihydrobenzo[d]oxazol-6-yl)piperidin-4-yl)methyl)piperazin-1-yl)-N-(trans-4-((5-fluoro-4-(3-(2-oxopyridin-1(2H)-yl)phenyl)pyrimidin-2-yl)amino)cyclohexyl)acetamide (Compound 360). To a solution of 3-(2-oxo-6-(4-(piperazin-1-ylmethyl)piperidin-1-yl)benzo[d]oxazol-3 (2H)-yl)piperidine-2,6-dione (60.0 mg, 140 μmol, 1.00 eq) and 2-bromo-N-(trans-4-((5-fluoro-4-(3-(2-oxopyridin-1(2H)-yl)phenyl)pyrimidin-2-yl)amino)cyclohexyl)acetamide (84.2 mg, 168 μmol, 1.20 eq) in DMF (2 mL) was added KI (46.6 mg, 280 μmol, 2.00 eq) and K₂CO₃ (38.7 mg, 280 μmol, 2.00 eq) at 20 °C. The mixture was stirred at 20 °C for 12 h. LC-MS showed the desired mass was detected. The reaction mixture was filtered and concentrated under reduced pressure. The residue was purified by prep-HPLC (column: Phenomenex Luna C18 100*30 mm*3 um; mobile phase: [H₂O (0.2% FA)-ACN]; gradient: 1%-40% B over 8.0 min) to give the title compound (Compound 360) (20 mg, 22.65 μmol, 16.1% yield, 95.9% purity) as a purple solid. LCMS (ES⁺): m/z 847.4 [M+H]⁺

### Example 354. Synthesis of trans-2-[4-[[1-[4-[(2,6-dioxo-3-piperidyl)amino]-2-fluoro-phenyl]-4-piperidyl]methyl]piperazin-1-yl]-N-[4-[[5-fluoro-4-[3-(2-oxo-1-pyridyl)phenyl]pyrimidin-2-yl]amino]cyclohexyl]acetamide (Compound 327)

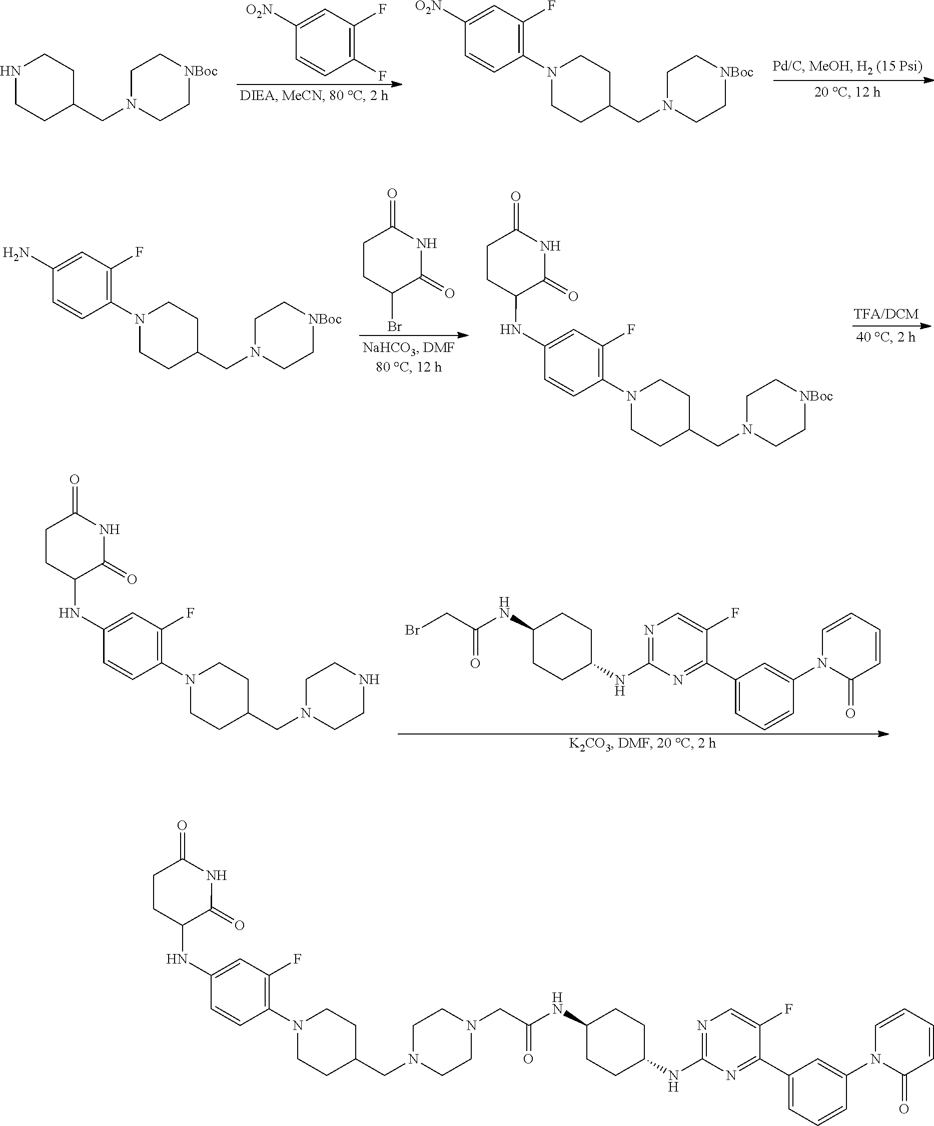

Synthesis of tert-butyl 4-[[1-(2-fluoro-4-nitro-phenyl)-4-piperidyl]methyl]piperazine-1-carboxylate. To a solution of tert-butyl 4-(4-piperidylmethyl) piperazine-1-carboxylate (7.00 g, 24.7 mmol, 1.00 eq) in MeCN (70 mL) was added DIEA (9.58 g, 74.1 mmol, 12.9 mL, 3.00 eq) and 1,2-difluoro-4-nitro-benzene (7.86 g, 49.4 mmol, 5.47 mL, 2.00 eq) at 20 °C. The mixture was stirred at 80 °C for 2 h. LC-MS showed the desired mass was detected. The solid was filtered, washed with EtOAc (3*20.0 mL), and dried under high vacuum to give the title compound (7.5 g, crude) as a yellow solid. LCMS (ES⁺): m/z 423.3 [M+H]⁺.

Synthesis of tert-butyl 4-[[1-(4-amino-2-fluoro-phenyl)-4-piperidyl]methyl]piperazine-1-carboxylate. A mixture of tert-butyl 4-[[1-(2-fluoro-4-nitro-phenyl)-4-piperidyl]methyl]piperazine-1-carboxylate (7.00 g, 16.5 mmol, 1.00 eq), 10% Pd/C (3.53 g, 16.5 mmol, 50% purity, 1.00 eq) in MeOH (2 mL) at 20 °C under N₂ atmosphere. The suspension was degassed and purged with H₂ for 3 times. The mixture was stirred under H₂ (15 Psi) at 20 °C for 12 h. LC-MS showed the desired mass was detected. The reaction mixture was filtered and the filtrate concentrated under high vacuum to give the title compound (6.50 g, crude) as a yellow oil. LCMS (ES⁺): m/z 393.2[M+H]⁺.

Synthesis of tert-butyl 4-[[1-[4-[(2,6-dioxo-3-piperidyl)amino]-2-fluoro-phenyl]-4-piperidyl]methyl]piperazine-1-carboxylate. To a solution of tert-butyl 4-[[1-(4-amino-2-fluoro-phenyl)-4-piperidyl]methyl]piperazine-1-carboxylate (4.00 g, 10.1 mmol, 1.00 eq) in DMF (40 mL) was added NaHCO₃ (2.57 g, 30.5 mmol, 1.19 mL, 3.00 eq) and 3-bromopiperidine-2,6-dione (2.94 g, 15.29 mmol, 1.50 eq) at 20 °C. The mixture was stirred at 80 °C for 12 h. LC-MS showed the desired mass was detected. The reaction mixture was quenched by addition H₂O 30 mL, and then extracted with EtOAc (100 mL*3). The combined organic layers were dried over Na₂SO₄, filtered and concentrated under reduced pressure. The residue was purified by column chromatography (SiO₂, Petroleum ether:Ethyl acetate=1:0 to 3:1) to give the title compound (9 g, crude) as a yellow oil. LCMS (ES⁺): m/z 504.4 [M+H]⁺.

Synthesis of 3-[3-fluoro-4-[4-(piperazin-1-ylmethyl)-1-piperidyl]anilino]piperidine-2,6-dione. To a solution of tert-butyl 4-[[1-[4-[(2,6-dioxo-3-piperidyl)amino]-2-fluoro-phenyl]-4-piperidyl]methyl]piperazine-1-carboxylate (3.00 g, 5.96 mmol, 1.00 eq) in DCM (3 mL) was added TFA (4.61 g, 40.3 mmol, 3.00 mL, 6.78 eq). The mixture was stirred at 40 °C for 2 h. LC-MS showed the desired mass was detected. The reaction mixture was concentrated under reduced pressure to give the title compound (3 g, crude, TFA) as a yellow oil. LCMS (ES⁺): m/z 404.3 [M+H]⁺.

Synthesis of trans-2-[4-[[1-[4-[(2,6-dioxo-3-piperidyl)amino]-2-fluoro-phenyl]-4-piperidyl]methyl]piperazin-1-yl]-N-[4-[[5-fluoro-4-[3-(2-oxo-1-pyridyl)phenyl]pyrimidin-2-yl]amino]cyclohexyl]acetamide (Compound 327). To a solution of 3-[3-fluoro-4-[4-(piperazin-1-ylmethyl)-1-piperidyl]anilino]piperidine-2,6-dione (105 mg, 202.8 μmol, 1.00 eq, TFA) in DMF (0.5 mL) was added K₂CO₃ (84.1 mg, 608.6 μmol, 3.00 eq) and trans-2-bromo-N-[4-[[5-fluoro-4-[3-(2-oxo-1-pyridyl)phenyl]pyrimidin-2-yl]amino]cyclohexyl]acetamide (101 mg, 202 μmol, 1.00 eq) at 20 °C. The mixture was stirred at 20 °C for 2 h. LC-MS showed the desired mass was detected. The reaction mixture was quenched by addition H₂O 10 mL, and then extracted with EtOAc (10 mL*3). The combined organic layers were dried over Na₂SO₄, filtered and concentrated under reduced pressure. The residue was purified by prep-HPLC (column: Phenomenex luna C18 100*40 mm*3 um; mobile phase: [H₂O (0.2% FA)-ACN]; gradient: 1%-30% B over 8.0 min) to give the title compound (Compound 327) (17.9 mg, 21.3 μmol, 10.5% yield, 99.7% purity, 0.3 FA) as an off-white solid. LCMS (ES⁺): m/z 823.4 [M+H]⁺.

### Example 355. Synthesis of trans-′-(1-(2,6-dioxopiperidin-3-yl)-3-methyl-2-oxo-2,3-dihydro-1H-benzo[d]imidazol-5-yl)-N-(4-((5-fluoro-4-(3-(2-oxopyridin-1(2H)-yl)phenyl)pyrimidin-2-yl)amino)cyclohexyl)-[1,4′-bipiperidine]-4-carboxamide (Compound 344)

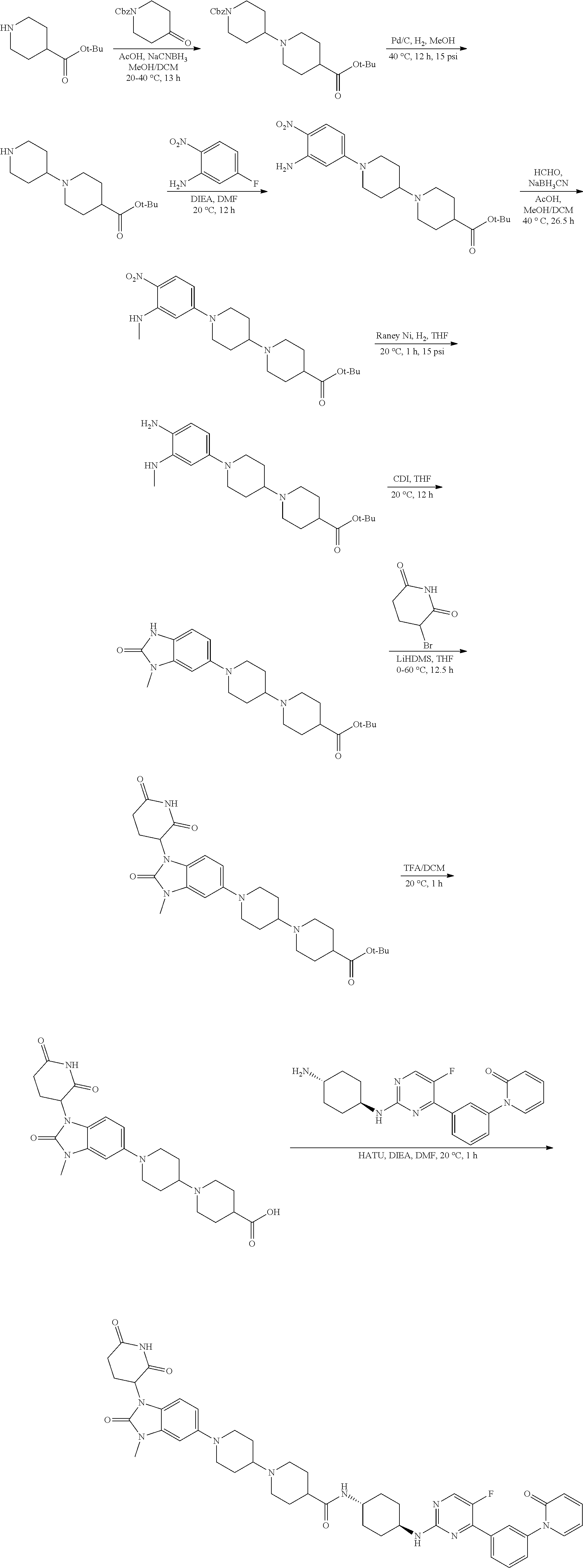

Synthesis of 1′-benzyl 4-(tert-butyl) [1,4′-bipiperidine]-1′,4-dicarboxylate. To a solution of tert-butyl piperidine-4-carboxylate (20.0 g, 90.2 mmol, 1.00 eq, HCl) in MeOH (120 mL) and DCM (200 mL) was added DIEA to adjust pH to 8, the mixture was stirred at 20 °C for 0.5 h, then CH₃COOH (5.42 g, 90.2 mmol, 5.16 mL, 1.00 eq) and benzyl 4-oxopiperidine-1-carboxylate (63.1 g, 271 mmol, 53.9 mL, 3.00 eq) were added to the mixture at 20 °C. The mixture was stirred at 40 °C for 0.5 h. Then NaBH₃CN (22.7 g, 361 mmol, 4.00 eq) was added to the mixture at 40 °C, then the mixture was stirred at 40 °C for 12 h. LCMS showed the desired mass was detected. H₂O (150 mL) was added to the mixture and the mixture was extracted with DCM (120 mL*3), the combined organic layers were washed with brine (120 mL*3), dried over Na₂SO₄, filtered and the filtrate was concentrated under reduced pressure. The residue was purified by MPLC (SiO₂, PE/EtOAc=1/1 to 0/1) to give the title compound (10.0 g, 24.8 mmol, 27.5% yield) as a colorless oil. LCMS (ES+): m/z 403.3 [M+H]⁺.

Synthesis of tert-butyl [1,4′-bipiperidine]-4-carboxylate. To a solution of 1′-benzyl 4-(tert-butyl) [1,4′-bipiperidine]-1′,4-dicarboxylate (5.00 g, 12.4 mmol, 1.00 eq) in MeOH (50 mL) was added 10% Pd/C (8 g, 50% purity) at 20 °C, the mixture was degassed and purged with H₂ for three times, the mixture was stirred at 40 °C for 12 h under H₂ (15 psi) atmosphere. LCMS showed 1′-benzyl 4-(tert-butyl) [1,4′-bipiperidine]-1′,4-dicarboxylate was consumed and the desired mass was detected. The mixture was filtered and the filtrate was concentrated under reduced pressure to give the title compound (3 g, crude) as a colorless oil. LCMS (ES⁺): m/z 269.2 [M+H]⁺.

Synthesis of tert-butyl 1′-(3-amino-4-nitrophenyl)-[1,4′-bipiperidine]-4-carboxylate. To a solution of tert-butyl [1,4′-bipiperidine]-4-carboxylate (2.50 g, 9.31 mmol, 1.00 eq) in DMF (40 mL) were added DIEA (3.61 g, 27.9 mmol, 4.87 mL, 3.00 eq) and 5-fluoro-2-nitroaniline (1.45 g, 9.31 mmol, 1.00 eq) at 20 °C. The mixture was stirred at 20 °C for 12 h. LCMS showed tert-butyl [1,4′-bipiperidine]-4-carboxylate was consumed completely and the desired mass was detected. To the mixture was added H₂O (50 mL), then the reaction mixture was filtered and the filter cake was dried under reduced pressure to give the title compound (2.33 g, crude) as a yellow solid. LCMS (ES⁺): m/z 405.3 [M+H]⁺.

Synthesis of tert-butyl 1′-(3-(methylamino)-4-nitrophenyl)-[1,4′-bipiperidine]-4-carboxylate. To a solution of tert-butyl 1′-(3-amino-4-nitrophenyl)-[1,4′-bipiperidine]-4-carboxylate (960 mg, 2.37 mmol, 1.00 eq) in DCM (10 mL) and MeOH (20 mL) were added AcOH (14.3 mg, 237 μmol, 13.6 μL, 0.100 eq) and HCHO (1.93 g, 23.7 mmol, 1.77 mL, 10.0 eq) at 20 °C and the mixture was stirred at 40 °C for 0.5 h. Then to the mixture was added NaBH₃CN (746 mg, 11.9 mmol, 5.00 eq) at 20 °C and the resulting mixture was stirred at 40 °C for 12 h. LCMS showed tert-butyl 1′-(3-amino-4-nitrophenyl)-[1,4′-bipiperidine]-4-carboxylate remained and the desired mass was detected. Then to the mixture was added HCHO (1.93 g, 23.7 mmol, 1.77 mL, 10.0 eq) at 20 °C and the mixture was stirred at 40 °C for 2 h. Then to the mixture was added NaBH₃CN (746 mg, 11.9 mmol, 5.00 eq) at 20 °C. The mixture was stirred at 40 °C for 12 h. LCMS showed tert-butyl 1′-(3-amino-4-nitrophenyl)-[1,4′-bipiperidine]-4-carboxylate was consumed completely and the desired mass was detected. The mixture was concentrated under reduced pressure. The mixture was diluted with H₂O (20 mL) and then extracted with DCM (20 mL*3). The combined organic layers were dried over Na₂SO₄, filtered and the filtrate was concentrated under reduced pressure. The residue was purified by MPLC (SiO₂, DCM/MeOH=1/0 to 10/1) to give the title compound (260 mg, crude) as a yellow oil. LCMS (ES⁺): m/z 419.2 [M+H]⁺.

Synthesis of tert-butyl 1′-(4-amino-3-(methylamino)phenyl)-[1,4′-bipiperidine]-4-carboxylate. To a stirred solution of tert-butyl 1′-(3-(methylamino)-4-nitrophenyl)-[1,4′-bipiperidine]-4-carboxylate (50.0 mg, 119 μmol, 1.00 eq) in THF (4 mL) was added Raney-Ni (50.0 mg) at 20 °C under N₂. The mixture was degassed and purged with H₂ for 3 times and the resulting mixture was stirred at 20 °C for 1 h under H₂ (15 psi). LCMS showed tert-butyl 1′-(3-(methylamino)-4-nitrophenyl)-[1,4′-bipiperidine]-4-carboxylate was consumed completely and the desired mass was detected. The mixture was filtered and the filtrate was concentrated under reduced pressure to give the title compound (50.0 mg, crude) as a yellow oil. LCMS (ES⁺): m/z 389.2 [M+H]⁺.

Synthesis of tert-butyl 1′-(3-methyl-2-oxo-2,3-dihydro-1H-benzo[d]imidazol-5-yl)-[1,4′-bipiperidine]-4-carboxylate. To a stirred solution of tert-butyl 1′-(4-amino-3-(methylamino)phenyl)-[1,4′-bipiperidine]-4-carboxylate (50.0 mg, 129 μmol, 1.00 eq) in THF (3 mL) was added CDI (31.3 mg, 193 μmol, 1.50 eq) at 0 °C. The resulting mixture was stirred at 20 °C for 12 h. LCMS showed tert-butyl 1′-(4-amino-3-(methylamino)phenyl)-[1,4′-bipiperidine]-4-carboxylate was consumed completely and the desired mass was detected. The mixture was diluted with H₂O (3 mL) and then extracted with EtOAc (3 mL*3). The combined organic layers were dried over Na₂SO₄, filtered and the filtrate was concentrated under reduced pressure to give a residue. The residue was purified by prep-TLC (SiO₂, EtOAc/MeOH=10/1) to give the title compound (50.0 mg, crude) as a yellow oil. LCMS (ES⁺): m/z 415.2 [M+H]⁺.

Synthesis of tert-butyl 1′-(1-(2,6-dioxopiperidin-3-yl)-3-methyl-2-oxo-2,3-dihydro-1H-benzo[d]imidazol-5-yl)-[1,4′-bipiperidine]-4-carboxylate. To a stirred solution of tert-butyl 1′-(3-methyl-2-oxo-2,3-dihydro-1H-benzo[d]imidazol-5-yl)-[1,4′-bipiperidine]-4-carboxylate (50.0 mg, 121 μmol, 1.00 eq) in THF (2 mL) was added LiHMDS (1 M, 362 μL, 3.00 eq) at 0 °C and the mixture was stirred at 0 °C for 0.5 h under N₂. To the mixture was added 3-bromopiperidine-2,6-dione (46.3 mg, 241 μmol, 2.00 eq) in THF (1 mL) at 20 °C and the resulting mixture was stirred at 60 °C for 12 h under N₂. LCMS showed tert-butyl 1′-(3-methyl-2-oxo-2,3-dihydro-1H-benzo[d]imidazol-5-yl)-[1,4′-bipiperidine]-4-carboxylate was consumed and the desired mass was detected. The reaction mixture was quenched by addition H₂O (5 mL, 0 °C) and then extracted with EtOAc (3 mL*3). The combined organic layers were dried over Na₂SO₄, filtered and the filtrate was concentrated under reduced pressure give a residue. The residue was purified by prep-TLC (SiO₂, EtOAc/MeOH=2/1) to give the title compound (50.0 mg, 95.1 μmol, 78.9% yield) as a yellow oil. LCMS (ES⁺): m/z 526.2 [M+H]⁺.

Synthesis of 1′-(1-(2,6-dioxopiperidin-3-yl)-3-methyl-2-oxo-2,3-dihydro-1H-benzo[d]imidazol-5-yl)-[1,4′-bipiperidine]-4-carboxylic acid. A solution of tert-butyl 1′-(1-(2,6-dioxopiperidin-3-yl)-3-methyl-2-oxo-2,3-dihydro-1H-benzo[d]imidazol-5-yl)-[1,4′-bipiperidine]-4-carboxylate (50.0 mg, 95.1 μmol, 1.00 eq) in DCM (1 mL) and TFA (0.5 mL) was stirred at 20 °C for 1 h. LCMS showed tert-butyl 1′-(1-(2,6-dioxopiperidin-3-yl)-3-methyl-2-oxo-2,3-dihydro-1H-benzo[d]imidazol-5-yl)-[1,4′-bipiperidine]-4-carboxylate was consumed completely and the desired mass was detected. The mixture was concentrated under reduced pressure to give the title compound (50.0 mg, crude) as a yellow oil. LCMS (ES⁺): m/z 470.1 [M+H]⁺.

Synthesis of trans-1′-(1-(2,6-dioxopiperidin-3-yl)-3-methyl-2-oxo-2,3-dihydro-1H-benzo[d]imidazol-5-yl)-N-(4-((5-fluoro-4-(3-(2-oxopyridin-1(2H)-yl)phenyl)pyrimidin-2-yl)amino)cyclohexyl)-[1,4′-bipiperidine]-4-carboxamide (Compound 344). To a stirred solution of 1′-(1-(2,6-dioxopiperidin-3-yl)-3-methyl-2-oxo-2,3-dihydro-1H-benzo[d]imidazol-5-yl)-[1,4′-bipiperidine]-4-carboxylic acid (50.0 mg, 106 μmol, 1.00 eq) and DIEA (41.3 mg, 319 μmol, 55.6 μL, 3.00 eq) in DMF (0.3 mL) were added HATU (60.7 mg, 160 μmol, 1.50 eq) and trans-1-(3-(2-((4-aminocyclohexyl)amino)-5-fluoropyrimidin-4-yl)phenyl)pyridin-2(1H)-one (35.4 mg, 85.2 μmol, 0.800 eq, HCl) at 20 °C. The resulting mixture was stirred at 20 °C for 1 h. LCMS showed 1′-(1-(2,6-dioxopiperidin-3-yl)-3-methyl-2-oxo-2,3-dihydro-1H-benzo[d]imidazol-5-yl)-[1,4′-bipiperidine]-4-carboxylic acid was consumed completely and the desired mass was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 25%-55% B over 8.0 min) to give the title compound (Compound 344) (7.8 mg, 9.35 μmol, 8.78% yield, 99.6% purity) as a white solid. LCMS (ES⁺): m/z 831.4 [M+H]⁺

### Example 356. Synthesis of trans-1′-(4-((2,6-dioxopiperidin-3-yl)amino)-2-fluorophenyl)-N-(4-((5-fluoro-4-(3-(2-oxopyridin-1(2H)-yl)phenyl)pyrimidin-2-yl)amino)cyclohexyl)-[1,4′-bipiperidine]-4-carboxamide (Compound 305)

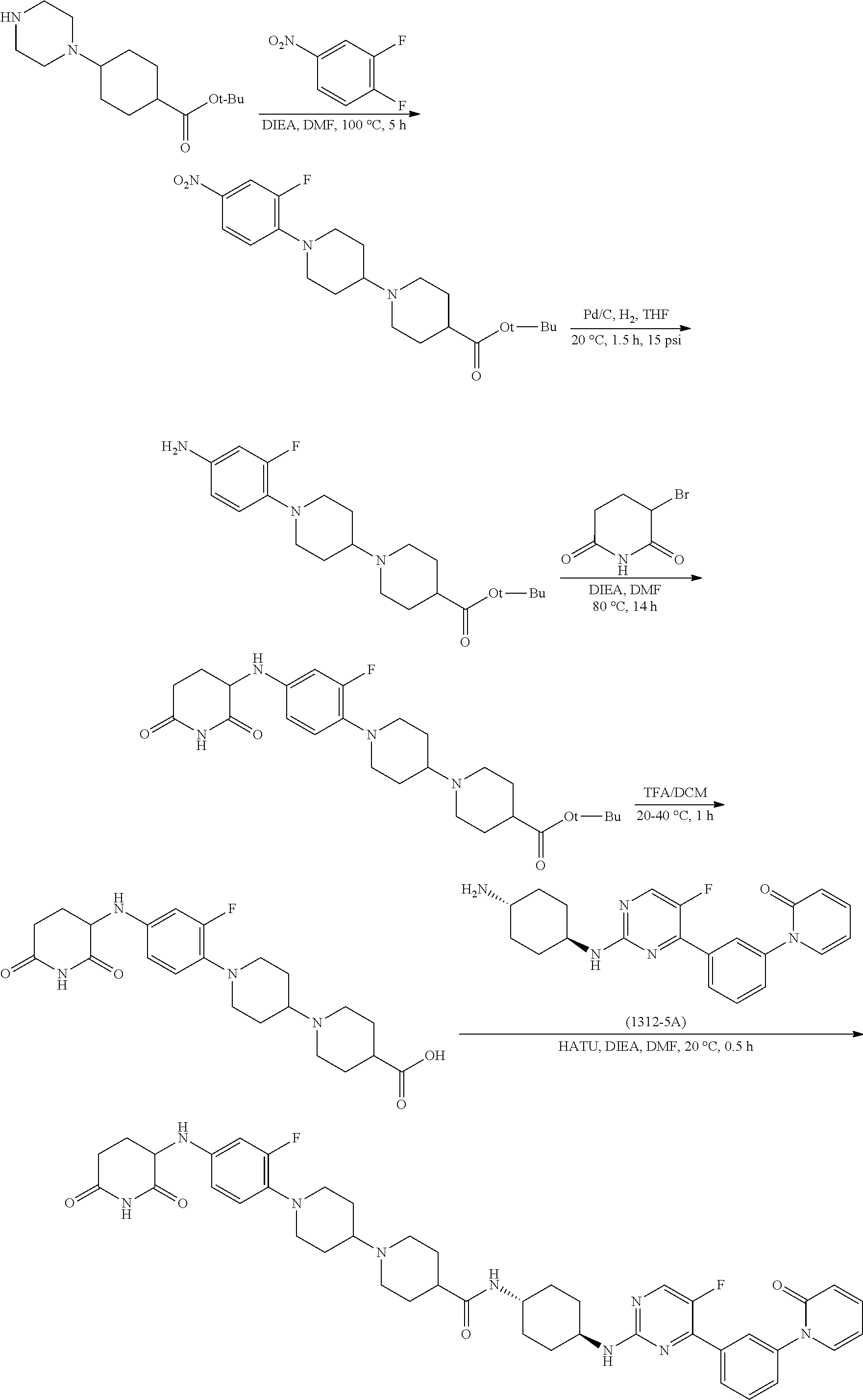

Synthesis of tert-butyl 1′-(2-fluoro-4-nitrophenyl)-[1,4′-bipiperidine]-4-carboxylate. To a solution of tert-butyl [1,4′-bipiperidine]-4-carboxylate (700 mg, 2.61 mmol, 1.00 eq) and 1,2-difluoro-4-nitrobenzene (415 mg, 2.61 mmol, 289 μL, 1.00 eq) in DMF (10 mL) was added DIEA (1.01 g, 7.82 mmol, 1.36 mL, 3.00 eq) at 20 °C. The mixture was stirred at 100 °C for 5 h. LCMS showed tert-butyl [1,4′-bipiperidine]-4-carboxylate was consumed and the desired target MS was detected. H₂O (10 mL) was added to the mixture, then the mixture was filtered, the filter cake was dried under reduced pressure to give the title compound (870 mg, crude) as a yellow solid. LCMS (ES⁺): m/z 408.2 [M+H]⁺

Synthesis of tert-butyl 1′-(4-amino-2-fluorophenyl)-[1,4′-bipiperidine]-4-carboxylate. To a solution of tert-butyl 1′-(2-fluoro-4-nitrophenyl)-[1,4′-bipiperidine]-4-carboxylate (870 mg, 2.14 mmol, 1.00 eq) in THF (20 mL) was added 10% Pd/C (650 mg, 50% purity) at 20 °C, the mixture was degassed and purged with H₂ for three times, the resulting mixture was stirred under H₂ (15 Psi) at 20 °C for 1.5 h. LCMS showed tert-butyl 1′-(2-fluoro-4-nitrophenyl)-[1,4′-bipiperidine]-4-carboxylate was consumed and the desired mass was detected. The mixture was filtered, the filtrate was concentrated under reduced pressure to give the title compound (900 mg, crude) as a brown oil. LCMS (ES⁺): m/z 378.2 [M+H]⁺

Synthesis of tert-butyl 1′-(4-((2,6-dioxopiperidin-3-yl)amino)-2-fluorophenyl)-[1,4′-bipiperidine]-4-carboxylate. To solution of tert-butyl 1′-(4-amino-2-fluorophenyl)-[1,4′-bipiperidine]-4-carboxylate (800 mg, 2.12 mmol, 1.00 eq) and 3-bromopiperidine-2,6-dione (814 mg, 4.24 mmol, 2.00 eq) in DMF (10 mL) was added DIEA (822 mg, 6.36 mmol, 1.11 mL, 3.00 eq) at 20 °C, the mixture was stirred at 80 °C for 12 h. LCMS showed tert-butyl 1′-(4-amino-2-fluorophenyl)-[1,4′-bipiperidine]-4-carboxylate remained and the desired mass was detected, then 3-bromopiperidine-2,6-dione (814 mg, 4.24 mmol, 2.00 eq) was added to the mixture at 20 °C, the mixture was stirred at 80 °C for another 2 h, LCMS showed tert-butyl 1′-(4-amino-2-fluorophenyl)-[1,4′-bipiperidine]-4-carboxylate was consumed mostly and the desired mass was detected. H₂O (10 mL) was added to the mixture and the mixture was extracted with EtOAc (10 mL*3), the combined organic layers were washed with brine (10 mL*3), dried over Na₂SO₄, filtered and the filtrate was concentrated under reduced pressure to give a residue. The residue was purified by MPLC (SiO₂, Petroleum ether/Ethyl acetate=1/0 to 0/1) to give the title compound (300 mg, 614 μmol, 29.0% yield) as a brown oil. LCMS (ES⁺): m/z 489.3 [M+H]⁺

Synthesis of 1′-(4-((2,6-dioxopiperidin-3-yl)amino)-2-fluorophenyl)-[1,4′-bipiperidine]-4-carboxylic acid. A solution of tert-butyl 1′-(4-((2,6-dioxopiperidin-3-yl)amino)-2-fluorophenyl)-[1,4′-bipiperidine]-4-carboxylate (180 mg, 368 μmol, 1.00 eq) in TFA (1 mL) and DCM (3 mL) was stirred at 20 °C for 0.5 h. LCMS showed tert-butyl 1′-(4-((2,6-dioxopiperidin-3-yl)amino)-2-fluorophenyl)-[1,4′-bipiperidine]-4-carboxylate remained and the desired mass was detected, then the mixture was stirred at 40 °C for another 0.5 h, LCMS showed tert-butyl 1′-(4-((2,6-dioxopiperidin-3-yl)amino)-2-fluorophenyl)-[1,4′-bipiperidine]-4-carboxylate was consumed and the desired mass was detected. The mixture was concentrated under reduced pressure to give the title compound (150 mg, crude) as a brown oil. LCMS (ES⁺): m/z 433.2 [M+H]⁺

Synthesis of trans-1′-(4-((2,6-dioxopiperidin-3-yl)amino)-2-fluorophenyl)-N-(4-((5-fluoro-4-(3-(2-oxopyridin-1(2H)-yl)phenyl)pyrimidin-2-yl)amino)cyclohexyl)-[1,4′-bipiperidine]-4-carboxamide (Compound 305). To a solution of 1′-(4-((2,6-dioxopiperidin-3-yl)amino)-2-fluorophenyl)-[1,4′-bipiperidine]-4-carboxylic acid (150 mg, 347 μmol, 1.00 eq) in DMF (3 mL) were added HATU (132 mg, 347 μmol, 1.00 eq) and DIEA (225 mg, 1.74 mmol, 303 μL, 5.00 eq) at 20 °C, then trans-1-(3-(2-((4-aminocyclohexyl)amino)-5-fluoropyrimidin-4-yl)phenyl)pyridin-2(1H)-one (120 mg, 243 μmol, 0.700 eq, TFA) was added to the mixture, the mixture was stirred at 20 °C for 0.5 h. LCMS showed 1′-(4-((2,6-dioxopiperidin-3-yl)amino)-2-fluorophenyl)-[1,4′-bipiperidine]-4-carboxylic acid was consumed and the desired mass was detected. The mixture was concentrated under reduced pressure to give a residue. The residue was purified by prep-HPLC (column: 3_Phenomenex Luna C18 75*30 mm*3 um; mobile phase: [H₂O (0.1% TFA)-ACN]; gradient: 5%-35% B over 8.0 min) to give the title compound (Compound 305) (6.10 mg, 7.39 μmol, 2.13% yield, 96.2% purity) as a white solid. LCMS (ES⁺): m/z 794.3 [M+H]⁺

### Example 357. Synthesis of trans-trans-4-(4-(4-((2,6-dioxopiperidin-3-yl)amino)-2-fluorophenyl)piperazin-1-yl)-N-(4-((5-fluoro-4-(3-(2-oxopyridin-1(2H)-yl)phenyl)pyrimidin-2-yl)amino)cyclohexyl)cyclohexane-1-carboxamide (Compound 319)

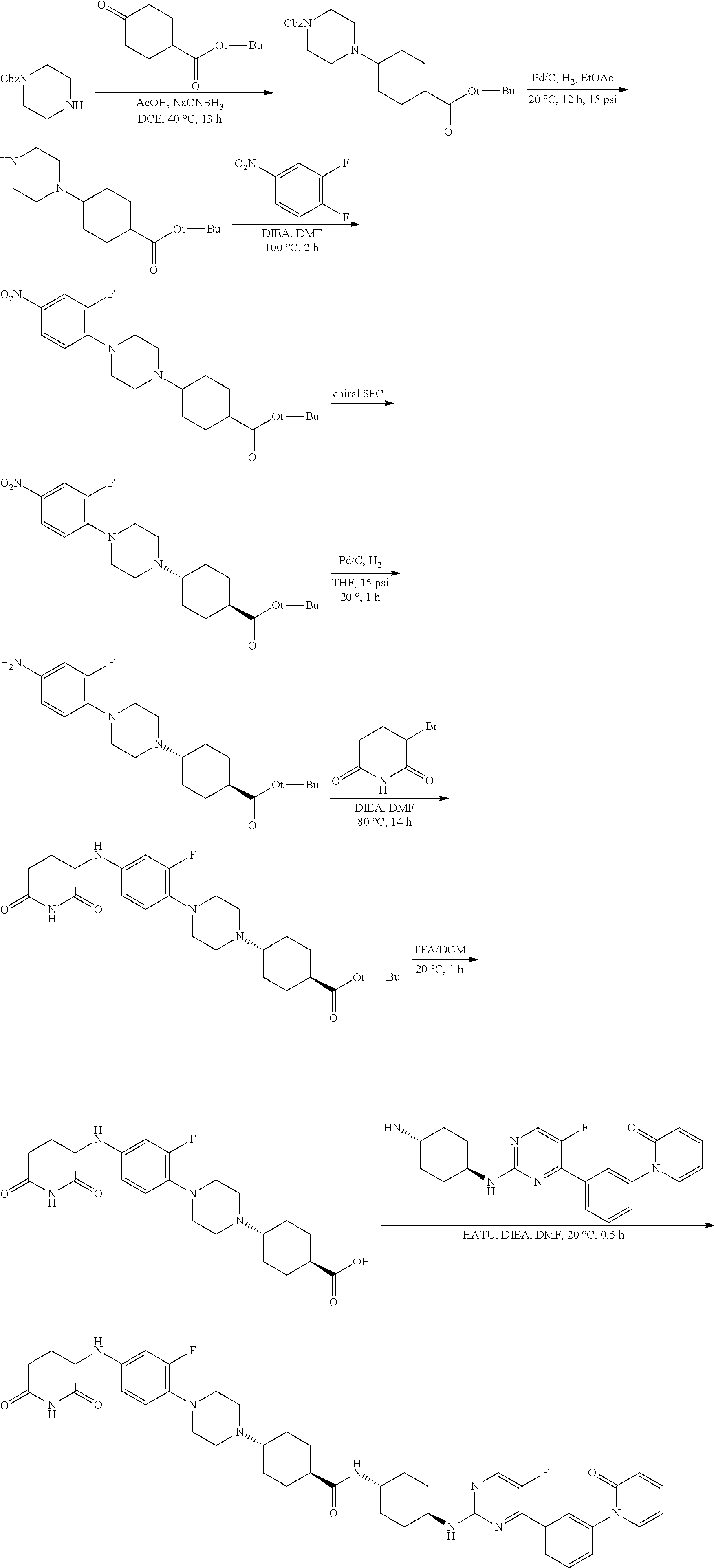

Synthesis of benzyl 4-(4-(tert-butoxycarbonyl)cyclohexyl)piperazine-1-carboxylate. To a solution of benzyl piperazine-1-carboxylate (5.00 g, 22.70 mmol, 4.38 mL, 1.00 eq) and tert-butyl 4-oxocyclohexane-1-carboxylate (13.5 g, 68.1 mmol, 3.00 eq) in DCE (80 mL) was added AcOH (136 mg, 2.27 mmol, 130 μL, 0.100 eq) at 20 °C. The mixture was stirred at 40 °C for 1 h. Then NaBH₃CN (2.85 g, 45.4 mmol, 2.00 eq) was added to the mixture, the resulting mixture was stirred at 40 °C for 12 h. LCMS showed the desired mass was detected. H₂O (100 mL) was added to the mixture and the mixture was extracted with DCM (50 mL*3), the combined organic layers were washed with brine (50 mL*3), dried over Na₂SO₄, filtered and the filtrate was concentrated under reduced pressure to give a residue. The residue was purified by MPLC (SiO₂, PE/EtOAc=1/0 to 0/1) to give the title compound (3.30 g, 8.20 mmol, 36.1% yield) as a colorless oil. LCMS (ES⁺): m/z 403.3 [M+H]⁺

Synthesis of tert-butyl 4-(piperazin-1-yl)cyclohexane-1-carboxylate. To a solution of benzyl 4-(4-(tert-butoxycarbonyl)cyclohexyl)piperazine-1-carboxylate (3.10 g, 7.70 mmol, 1.00 eq) in EtOAc (50 mL) was added 10% Pd/C (300 mg, 50% purity) at 20 °C, the mixture was degassed and purged with H₂ for three times, then the resulting mixture was stirred at 20 °C for 12 h under H₂ (15 psi). LCMS showed benzyl 4-(4-(tert-butoxycarbonyl)cyclohexyl)piperazine-1-carboxylate was consumed and the desired mass was detected. The mixture was filtered and the filtrate was concentrated under reduced pressure to give the title compound (1.70 g, crude) as a yellow oil. LCMS (ES⁺): m/z 269.2 [M+H]⁺

Synthesis of tert-butyl 4-(4-(2-fluoro-4-nitrophenyl)piperazin-1-yl)cyclohexane-1-carboxylate. To a solution of tert-butyl 4-(piperazin-1-yl)cyclohexane-1-carboxylate (1.70 g, 6.33 mmol, 1.00 eq) and 1,2-difluoro-4-nitro-benzene (1.01 g, 6.33 mmol, 701 μL, 1.00 eq) in DMF (10 mL) was added DIEA (2.46 g, 19.0 mmol, 3.31 mL, 3.00 eq) at 20 °C. Then the mixture was stirred at 100 °C for 2 h. LCMS showed tert-butyl 4-(piperazin-1-yl)cyclohexane-1-carboxylate was consumed and the desired mass was detected. H₂O (10 mL) was added to the mixture, then the mixture was filtered, the filter cake was dried under reduced pressure to give the title compound (2.10 g, crude) as a yellow solid. LCMS (ES⁺): m/z 408.3 [M+H]⁺

Synthesis of trans-tert-butyl 4-(4-(2-fluoro-4-nitrophenyl)piperazin-1-yl)cyclohexane-1-carboxylate. tert-butyl 4-(4-(2-fluoro-4-nitrophenyl)piperazin-1-yl)cyclohexane-1-carboxylate (2.10 g, crude) was separated by chiral SFC (CAS-TJ-ANA-SFC-C (Waters UPCC with PDA)); Column: Chiralpak IC-3, 100×4.6 mm LD., 3 um; Mobile phase: A: CO₂ B: MeOH (0.2% NH₃ (7M in MeOH), v/v; Gradient: A:B=50:50; Flow rate: 3.4 mL/min; Column temp.: 35 °C; to give the title compound (890 mg, 2.18 mmol, 34.5% yield) as a yellow solid. LCMS (ES⁺): m/z 408.3 [M+H]⁺

Synthesis of trans-tert-butyl 4-(4-(4-amino-2-fluorophenyl)piperazin-1-yl)cyclohexane-1-carboxylate. To a mixture of trans-tert-butyl 4-(4-(2-fluoro-4-nitrophenyl)piperazin-1-yl)cyclohexane-1-carboxylate (250 mg, 614 umol, 1.00 eq) in THF (6 mL) was added 10% Pd/C (500 mg, 50% purity) at 20 °C, the mixture was degassed and purged with H₂, the resulting mixture was stirred under H₂ (15 psi) at 20 °C for 1 h. LCMS showed trans-tert-butyl 4-(4-(2-fluoro-4-nitrophenyl)piperazin-1-yl)cyclohexane-1-carboxylate was consumed and the desired mass was detected. The mixture was filtered, the filtrate was concentrated under reduced pressure to give the title compound (230 mg, crude) as a yellow oil. LCMS (ES⁺): m/z 378.2 [M+H]⁺

Synthesis of trans-tert-butyl 4-(4-(4-((2,6-dioxopiperidin-3-yl)amino)-2-fluorophenyl)piperazin-1-yl)cyclohexane-1-carboxylate. To solution of trans-tert-butyl 4-(4-(4-amino-2-fluorophenyl)piperazin-1-yl)cyclohexane-1-carboxylate (230 mg, 609 μmol, 1.00 eq) and 3-bromopiperidine-2,6-dione (351 mg, 1.83 mmol, 3.00 eq) in DMF (5 mL) was added DIEA (236 mg, 1.83 mmol, 318 μL, 3.00 eq) at 20 °C, the mixture was stirred at 80 °C for 12 h. LCMS showed trans-tert-butyl 4-(4-(4-amino-2-fluorophenyl)piperazin-1-yl)cyclohexane-1-carboxylate remained and the desired mass was detected, then 3-bromopiperidine-2,6-dione (234 mg, 1.22 mmol, 2.00 eq) was added to the mixture, the mixture was stirred at 80 °C for another 2 h, LCMS showed trans-tert-butyl 4-(4-(4-amino-2-fluorophenyl)piperazin-1-yl)cyclohexane-1-carboxylate was consumed and the desired mass was detected. H₂O (5 mL) was added to the mixture and the mixture was extracted with EtOAc (5 mL*3), the combined organic layers were washed with brine (5 mL*3), dried over Na₂SO₄, filtered and the filtrate was concentrated under reduced pressure to give a residue. The residue was purified by prep-TLC (SiO₂, PE/THF=0/1) to give the title compound (240 mg, 491 umol, 80.6% yield) as a brown solid. LCMS (ES⁺): m/z 489.3 [M+H]⁺

Synthesis of trans-4-(4-(4-((2,6-dioxopiperidin-3-yl)amino)-2-fluorophenyl)piperazin-1-yl)cyclohexane-1-carboxylic acid. A mixture of trans-tert-butyl 4-(4-(4-((2,6-dioxopiperidin-3-yl)amino)-2-fluorophenyl)piperazin-1-yl)cyclohexane-1-carboxylate (150 mg, 307 umol, 1.00 eq) in DCM (0.6 mL) and TFA (0.2 mL) was stirred at 20 °C for 1 h, LCMS showed trans-tert-butyl 4-(4-(4-((2,6-dioxopiperidin-3-yl)amino)-2-fluorophenyl)piperazin-1-yl)cyclohexane-1-carboxylate was consumed and the desired mass was detected. The mixture was concentrated under reduced pressure to give the title compound (135 mg, crude) as a brown oil. LCMS (ES⁺): m/z 433.2 [M+H]⁺

Synthesis of trans-trans-4-(4-(4-((2,6-dioxopiperidin-3-yl)amino)-2-fluorophenyl)piperazin-1-yl)-N-(4-((5-fluoro-4-(3-(2-oxopyridin-1(2H)-yl)phenyl)pyrimidin-2-yl)amino)cyclohexyl)cyclohexane-1-carboxamide (Compound 319). To a solution of trans-4-(4-(4-((2,6-dioxopiperidin-3-yl)amino)-2-fluorophenyl)piperazin-1-yl)cyclohexane-1-carboxylic acid (150 mg, 347 μmol, 1.00 eq) in DMF (3 mL) were added HATU (132 mg, 347 μmol, 1.00 eq) and DIEA (224 mg, 1.74 mmol, 303 μL, 5.00 eq) at 20 °C, then trans-1-(3-(2-((4-aminocyclohexyl)amino)-5-fluoropyrimidin-4-yl)phenyl)pyridin-2(1H)-one (101 mg, 243 μmol, 0.700 eq, HCl) was added to the mixture, the mixture was stirred at 20 °C for 0.5 h. LCMS showed trans-4-(4-(4-((2,6-dioxopiperidin-3-yl)amino)-2-fluorophenyl)piperazin-1-yl)cyclohexane-1-carboxylic acid was consumed and the desired mass was detected. The mixture was concentrated under reduced pressure to give a residue. The residue was purified by prep-HPLC (column: Phenomenex Luna C18 80*30 mm*3 um; mobile phase: [H₂O (0.1% TFA)-ACN]; gradient: 10%-40% B over 8.0 min) to give the title compound (Compound 319) (54.4 mg, 66.6 μmol, 19.2% yield, 97.2% purity) as a brown solid. LCMS (ES⁺): m/z 794.4 [M+H]⁺

### Example 358. Synthesis of trans-(1R,4R)-4-(4-(4-(((R)-2,6-dioxopiperidin-3-yl)amino)-2-fluorophenyl)piperazin-1-yl)-N-((1r,4R)-4-((5-fluoro-4-(3-(2-oxopyridin-1(2H)-yl)phenyl)pyrimidin-2-yl)amino)cyclohexyl)cyclohexane-1-carboxamide (Compound 602) and trans-(1S,4R)-4-(4-(4-(((S)-2,6-dioxopiperidin-3-yl)amino)-2-fluorophenyl)piperazin-1-yl)-N-((1r,4R)-4-((5-fluoro-4-(3-(2-oxopyridin-1(2H)-yl)phenyl)pyrimidin-2-yl)amino)cyclohexyl)cyclohexane-1-carboxamide (Compound 603) (The Two Isomers were Assigned Arbitrarily)

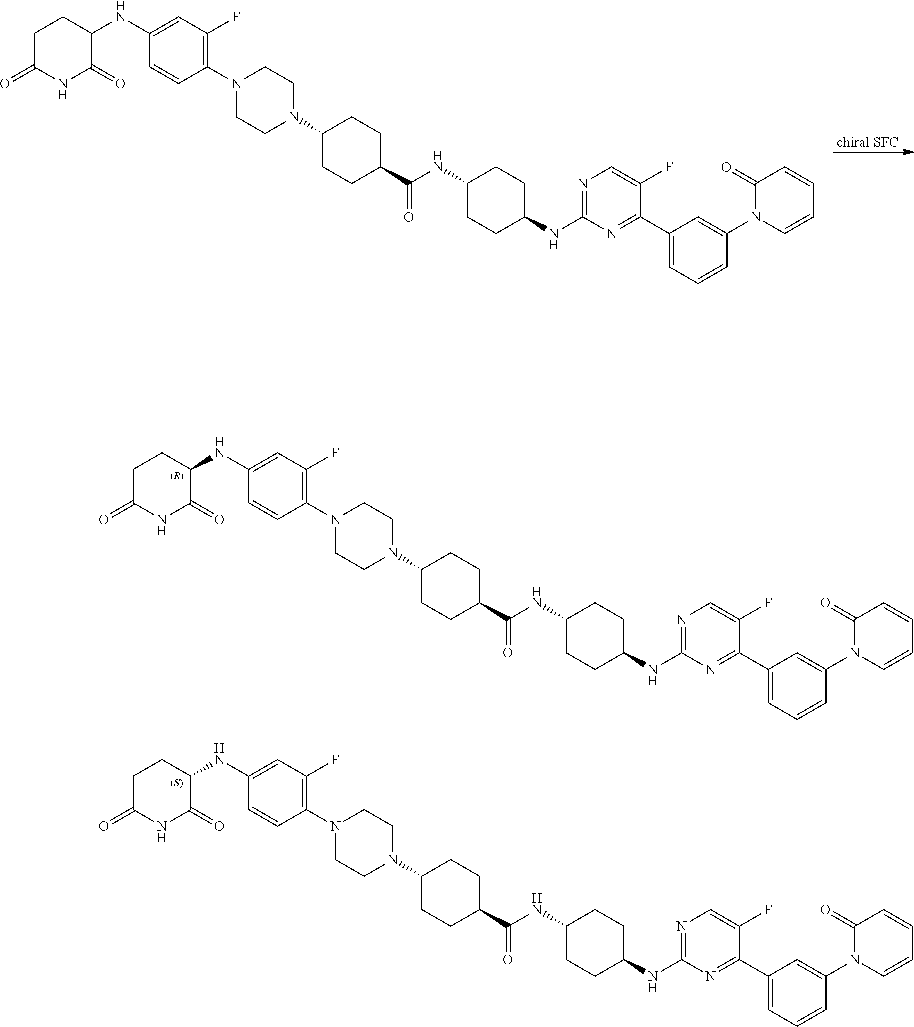

Synthesis of trans-(1R,4R)-4-(4-(4-(((R)-2,6-dioxopiperidin-3-yl)amino)-2-fluorophenyl)piperazin-1-yl)-N-((1r,4R)-4-((5-fluoro-4-(3-(2-oxopyridin-1(2H)-yl)phenyl)pyrimidin-2-yl)amino)cyclohexyl)cyclohexane-1-carboxamide (Compound 602) and trans-(1S,4R)-4-(4-(4-(((S)-2,6-dioxopiperidin-3-yl)amino)-2-fluorophenyl)piperazin-1-yl)-N-((1r,4R)-4-((5-fluoro-4-(3-(2-oxopyridin-1(2H)-yl)phenyl)pyrimidin-2-yl)amino)cyclohexyl)cyclohexane-1-carboxamide (Compound 603) (the two isomers were assigned arbitrarily). Trans-(1r,4R)-4-(4-(4-((2,6-dioxopiperidin-3-yl)amino)-2-fluorophenyl)piperazin-1-yl)-N-((1r,4R)-4-((5-fluoro-4-(3-(2-oxopyridin-1(2H)-yl)phenyl)pyrimidin-2-yl)amino)cyclohexyl)cyclohexane-1-carboxamide (Compound 319) (1.00 g, 1.26 mmol, 1.00 eq) was separated by chiral SFC (column: REGIS(S,S)WHELK-O1 (250 mm*25 mm, 10 um); mobile phase: [CO₂—IPA:ACN=1:1 (0.1% IPAm)]; B %: 56%, isocratic elution mode) to give the title compound (Compound 602) (384 mg, 459 μmol, 36.4% yield, 94.8% purity, 96.1% ee) (retention time: 3.283, peak 1) as an off white solid. LCMS (ES⁺): m/z 794.4 [M+H]⁺ and the title compound (Compound 603) (253 mg, 300 mol, 23.8% yield, 94.3% purity, 94.1% ee) (retention time: 3.963, peak 2) as a yellow solid. LCMS (ES⁺): m/z 794.4 [M+H]⁺

### Example 359. Synthesis of trans-4-(4-(4-((2,6-dioxopiperidin-3-yl)amino)-2-fluorophenyl)piperidin-1-yl)-N-(trans-4-((5-fluoro-4-(3-(2-oxopyridin-1(2H)-yl)phenyl)pyrimidin-2-yl)amino)cyclohexyl)cyclohexane-1-carboxamide (Compound 345)

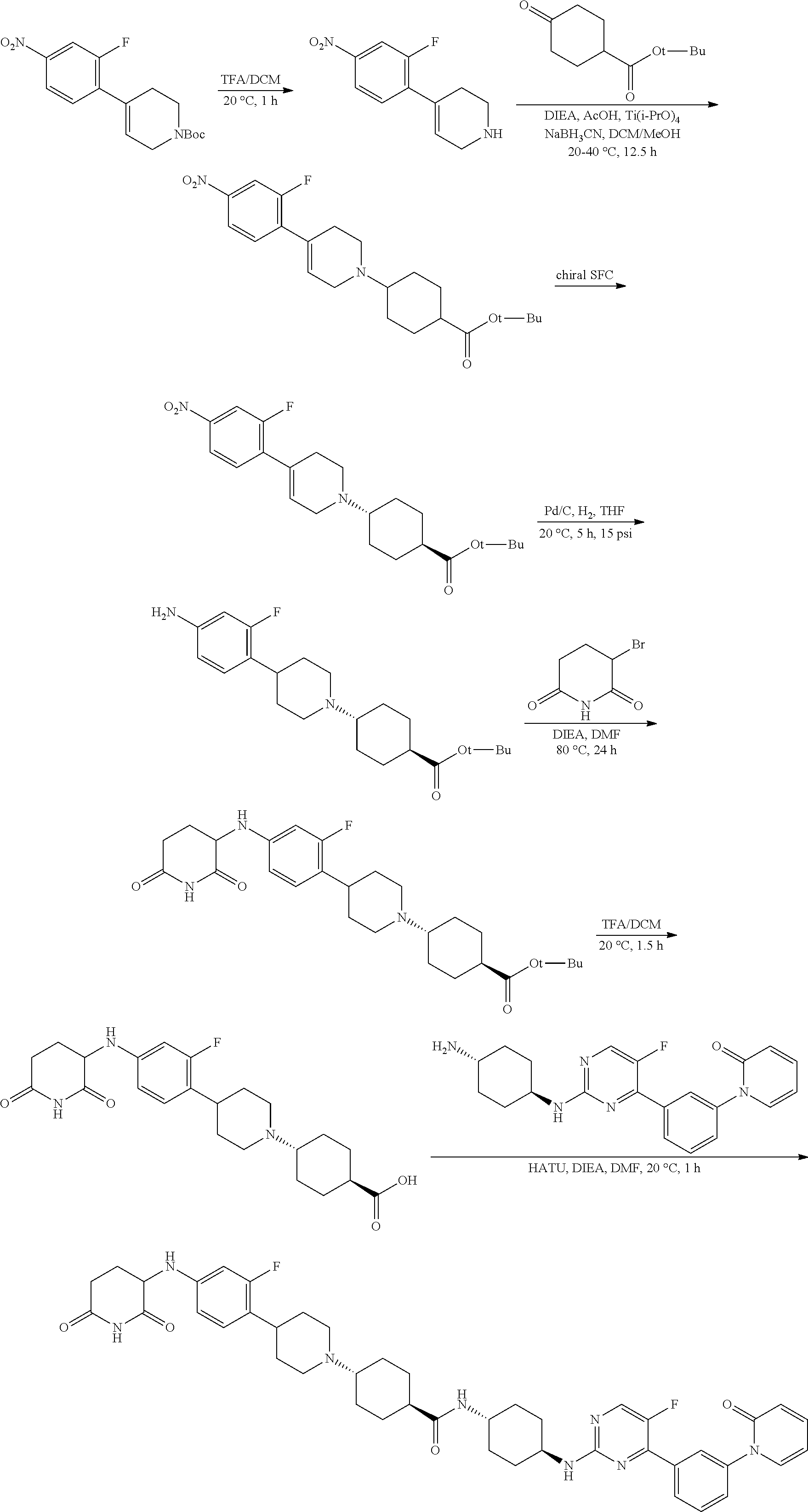

Synthesis of 4-(2-fluoro-4-nitrophenyl)-1,2,3,6-tetrahydropyridine. A solution of tert-butyl 4-(2-fluoro-4-nitrophenyl)-3,6-dihydropyridine-1(2H)-carboxylate (4.00 g, 12.4 mmol, 1.00 eq) in DCM (21 mL) and TFA (7 mL) was stirred at 20 °C for 1 h. LCMS showed tert-butyl 4-(2-fluoro-4-nitrophenyl)-3,6-dihydropyridine-1(2H)-carboxylate was consumed and the desired mass was detected. The reaction mixture was concentrated under reduced pressure to give the title compound (4.20 g, crude, TFA) as a yellow oil. LCMS (ES⁺): m/z 223.1 [M+H]⁺

Synthesis of tert-butyl 4-(4-(2-fluoro-4-nitrophenyl)-3,6-dihydropyridin-1(2H)-yl)cyclohexane-1-carboxylate. To a solution of 4-(2-fluoro-4-nitrophenyl)-1,2,3,6-tetrahydropyridine (4.20 g, 12.5 mmol, 1.00 eq, TFA) in MeOH (100 mL) and DCM (100 mL) was added DIEA to adjust pH to 8 at 20 °C. The mixture was stirred at 20 °C for 15 min. To the reaction was added tert-butyl 4-oxocyclohexane-1-carboxylate (19.8 g, 99.9 mmol, 8.00 eq), Ti(i-PrO)₄ (3.55 g, 12.5 mmol, 3.69 mL, 1.00 eq) and AcOH (3.75 g, 62.5 mmol, 3.58 μL, 5.00 eq) at 20 °C, pH was 5 now. The mixture was stirred at 40 °C for 15 min. To the reaction mixture was added NaBH₃CN (2.35 g, 37.5 mmol, 3.00 eq) at 20 °C and the resulting mixture was stirred at 40 °C for 12 h. LCMS showed 4-(2-fluoro-4-nitrophenyl)-1,2,3,6-tetrahydropyridine was consumed and the desired mass was detected. The mixture was concentrated under reduced pressure to give a residue. To the residue was added H₂O (200 mL) and then the residue was extracted with DCM (100 mL*3), the combined organic layers were washed with brine (200 mL*3), dried over Na₂SO₄, filtered and the filtrate was concentrated under reduced pressure to give a residue. The residue was purified by MPLC (SiO₂, PE/EtOAc=1/0 to 1/1) to give the title compound (4.00 g, 9.89 mmol, 79.2% yield) as a yellow oil. LCMS (ES⁺): m/z 405.1 [M+H]⁺

Synthesis of trans-tert-butyl 4-(4-(2-fluoro-4-nitrophenyl)-3,6-dihydropyridin-1(2H)-yl)cyclohexane-1-carboxylate. tert-butyl 4-(4-(2-fluoro-4-nitrophenyl)-3,6-dihydropyridin-1(2H)-yl)cyclohexane-1-carboxylate (4.00 g, 9.89 mmol, 1.00 eq) was separated by chiral SFC (column: DAICEL CHIRALPAK IG (250 mm*30 mm, 10 um); mobile phase: [CO₂-EtOH (0.1% NH₃H₂O)]; B %: 25%, isocratic elution mode) to give the title compound (837 mg, 2.07 mmol, 16.6% yield) as a yellow solid. LCMS (ES⁺): m/z 405.1 [M+H]⁺

Synthesis of trans-tert-butyl-4-(4-(4-amino-2-fluorophenyl)piperidin-1-yl)cyclohexane-1-carboxylate. To a solution of trans-tert-butyl 4-(4-(2-fluoro-4-nitrophenyl)-3,6-dihydropyridin-1(2H)-yl)cyclohexane-1-carboxylate (280 mg, 692 μmol, 1.00 eq) in THF (5 mL) was added 10% Pd/C (600 mg, 50% purity) at 20 °C under N₂, The mixture was degassed and purged with H₂ for 3 times and the mixture was stirred at 20 °C for 2 h under H₂ (15 psi). LCMS showed trans-tert-butyl 4-(4-(2-fluoro-4-nitrophenyl)-3,6-dihydropyridin-1(2H)-yl)cyclohexane-1-carboxylate remained and the desired target MS was detected. Then the mixture was stirred at 20 °C for another 3 h under H₂ (15 psi). LCMS showed trans-tert-butyl 4-(4-(2-fluoro-4-nitrophenyl)-3,6-dihydropyridin-1(2H)-yl)cyclohexane-1-carboxylate was consumed completely and the desired target MS was detected. The mixture was filtered, the filtrate was concentrated under reduced pressure to give the title compound (200 mg, crude) as a yellow oil. LCMS (ES⁺): m/z 377.3 [M+H]⁺

Synthesis of trans-tert-butyl-4-(4-(4-((2,6-dioxopiperidin-3-yl)amino)-2-fluorophenyl)piperidin-1-yl)cyclohexane-1-carboxylate. To a solution of trans-tert-butyl-4-(4-(4-amino-2-fluorophenyl)piperidin-1-yl)cyclohexane-1-carboxylate (200 mg, 531 μmol, 1.00 eq) and 3-bromopiperidine-2,6-dione (204 mg, 1.06 mmol, 2.00 eq) in DMF (6 mL) was added DIEA (275 mg, 2.12 mmol, 370 μL, 4.00 eq) at 20 °C. The mixture was stirred at 80 °C for 12 h. LCMS showed trans-tert-butyl-4-(4-(4-amino-2-fluorophenyl)piperidin-1-yl)cyclohexane-1-carboxylate remained and the desired target MS was detected. Then 3-bromopiperidine-2,6-dione (102 mg, 531 μmol, 1.00 eq) was added to the mixture at 20 °C, the mixture was stirred at 80 °C for another 12 h. LCMS showed trans-tert-butyl-4-(4-(4-amino-2-fluorophenyl)piperidin-1-yl)cyclohexane-1-carboxylate was consumed completely and the desired target MS was detected. H₂O (10 mL) was added to the mixture and the mixture was extracted with EtOAc (10 mL*3), the combined organic layers were washed with brine (10 mL*3), dried over Na₂SO₄, filtered and the filtrate was concentrated under reduced pressure to give a residue. The residue was purified by prep-TLC (SiO₂, EtOAc/MeOH=5/1) to give the title compound (50.0 mg, 102 μmol, 19.3% yield) as a yellow oil. LCMS (ES⁺): m/z 488.2 [M+H]⁺

Synthesis of trans-4-(4-(4-((2,6-dioxopiperidin-3-yl)amino)-2-fluorophenyl)piperidin-1-yl)cyclohexane-1-carboxylic acid. A solution of trans-tert-butyl-4-(4-(4-((2,6-dioxopiperidin-3-yl)amino)-2-fluorophenyl)piperidin-1-yl)cyclohexane-1-carboxylate (50.0 mg, 103 μmol, 1.00 eq) in DCM (0.5 mL) and TFA (0.1 mL) was stirred at 20 °C for 1.5 h. LCMS showed trans-tert-butyl-4-(4-(4-((2,6-dioxopiperidin-3-yl)amino)-2-fluorophenyl)piperidin-1-yl)cyclohexane-1-carboxylate was consumed completely and the desired target MS was detected. The mixture was concentrated under reduced pressure to give the title compound (50.0 mg, crude) as a yellow oil. LCMS (ES⁺): m/z 432.2 [M+H]⁺

Synthesis of trans-4-(4-(4-((2,6-dioxopiperidin-3-yl)amino)-2-fluorophenyl)piperidin-1-yl)-N-(trans-4-((5-fluoro-4-(3-(2-oxopyridin-1(2H)-yl)phenyl)pyrimidin-2-yl)amino)cyclohexyl)cyclohexane-1-carboxamide (Compound 345). To a solution of trans-4-(4-(4-((2,6-dioxopiperidin-3-yl)amino)-2-fluorophenyl)piperidin-1-yl)cyclohexane-1-carboxylic acid (50.0 mg, 116 μmol, 1.00 eq) in DMF (2 mL) were added HATU (35.3 mg, 92.7 μmol, 0.800 eq) and DIEA (74.8 mg, 580 μmol, 101 μL, 5.00 eq) at 20 °C, then trans-1-(3-(2-((4-aminocyclohexyl)amino)-5-fluoropyrimidin-4-yl)phenyl)pyridin-2(1H)-one (19.3 mg, 46.4 μmol, 0.400 eq, HCl) was added to the mixture, the mixture was stirred at 20 °C for 1 h. LCMS showed trans-4-(4-(4-((2,6-dioxopiperidin-3-yl)amino)-2-fluorophenyl)piperidin-1-yl)cyclohexane-1-carboxylic acid was consumed and the desired target MS was detected. The mixture was filtered and the filtrate was concentrated under reduced pressure to give a residue. The residue was purified by prep-HPLC (column: Phenomenex Luna C18 75*30 mm*3 um; mobile phase: [H₂O (0.1% TFA)-ACN]; gradient: 35%-55% B over 8.0 min) to give the title compound (Compound 345) (5.60 mg, 7.06 μmol, 6.09% yield, 100% purity) as a yellow solid. LCMS (ES⁺): m/z 793.4 [M+H]⁺

### Example 360. Synthesis of (1R,4R)-4-(4-(4-(((R)-2,6-dioxopiperidin-3-yl)amino)-2-fluorophenyl)piperidin-1-yl)-N-((1r,4R)-4-((5-fluoro-4-(3-(2-oxopyridin-1(2H)-yl)phenyl)pyrimidin-2-yl)amino)cyclohexyl)cyclohexane-1-carboxamide (Compound 706) and (1S,4R)-4-(4-(4-(((S)-2,6-dioxopiperidin-3-yl)amino)-2-fluorophenyl)piperidin-1-yl)-N-((1r,4R)-4-((5-fluoro-4-(3-(2-oxopyridin-1(2H)-yl)phenyl)pyrimidin-2-yl)amino)cyclohexyl)cyclohexane-1-carboxamide (Compound 457) (The Two Isomers were Assigned Arbitrarily)

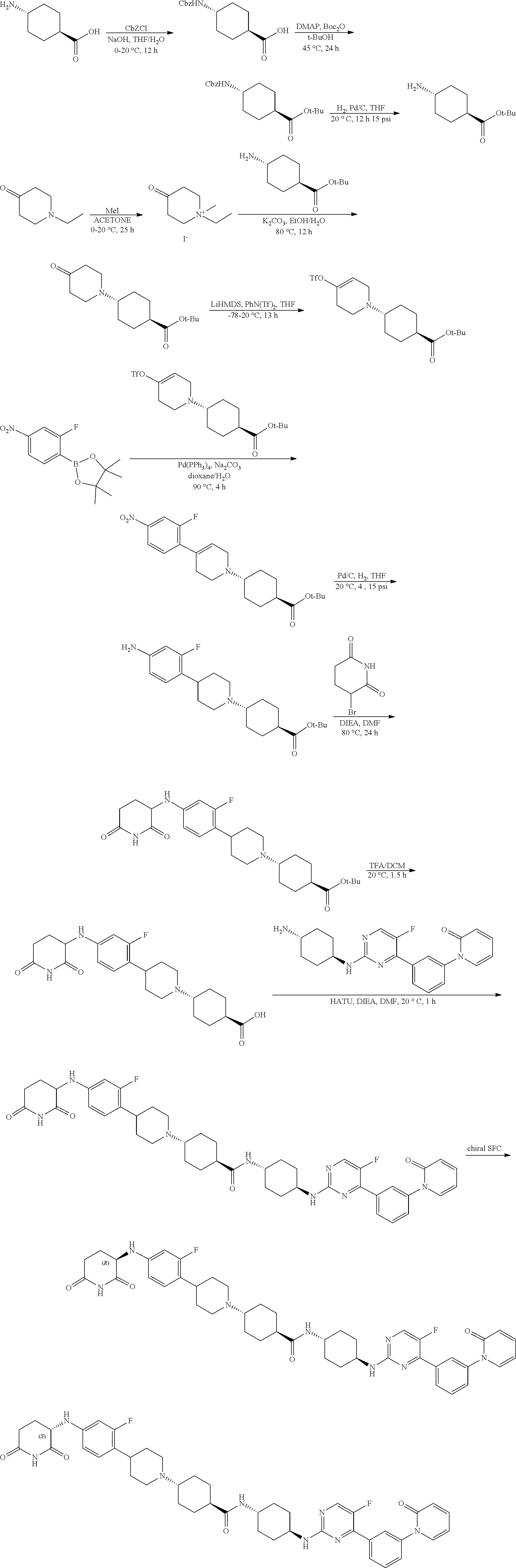

Synthesis of trans-4-(((benzyloxy)carbonyl)amino)cyclohexane-1-carboxylic acid. To a stirred solution of trans-4-aminocyclohexane-1-carboxylic acid (100 g, 700 mmol, 1.00 eq) in THF (1200 mL) was added a solution of NaOH (56.0 g, 1.40 μmol, 2.00 eq) in H₂O (2400 mL) at 20 °C, then CbzCl (131 g, 770 mmol, 110 mL, 1.10 eq) was added to the mixture at 0 °C, the mixture was stirred at 20 °C for 12 h. LCMS showed trans-4-aminocyclohexane-1-carboxylic acid was consumed and the desired target MS was detected. The mixture was concentrated under reduced pressure to give a residue. To the residue was added HCl (1 M) to adjust pH=4, then the mixture was filtered, the filter cake was dried under reduced pressure to give the title compound (150 g, crude) as a white solid. LCMS (ES⁺): m/z 278.2 [M+H]⁺

Synthesis of trans-tert-butyl 4-(((benzyloxy)carbonyl)amino)cyclohexane-1-carboxylate. To a solution of trans-4-(((benzyloxy)carbonyl)amino)cyclohexane-1-carboxylic acid (110 g, 397 mmol, 1.00 eq) in t-BuOH (1200 mL) were added DMAP (24.2 g, 199 mmol, 0.500 eq) and (Boc)₂O (347 g, 1.59 μmol, 365 mL, 4.00 eq) at 20 °C, the mixture was stirred at 45 °C for 12 h. LCMS showed trans-4-(((benzyloxy)carbonyl)amino)cyclohexane-1-carboxylic acid remained and the desired target MS was detected. To the mixture was added DMAP (9.69 g, 79.4 mmol, 0.200 eq) and (Boc)₂O (173 g, 794 mmol, 182 mL, 2.00 eq) at 20 °C, the mixture was stirred at 45 °C for another 12 h. LCMS showed trans-4-(((benzyloxy)carbonyl)amino)cyclohexane-1-carboxylic acid was consumed completely and the desired target MS was detected. The mixture was concentrated under reduced pressure to give a residue. H₂O (2000 mL) was added to the residue and the mixture was extracted with EtOAc (1000 mL*3), then the combined organic layers were dried over Na₂SO₄, filtered and the filtrate was concentrated under reduced pressure to give a residue. The residue was purified by MPLC (SiO₂, PE/EtOAc=1/0 to 10/1) to give the title compound (90.0 g, 270 mmol, 68.0% yield) as a colorless oil. LCMS (ES⁺): m/z 278.2 [M+H−56]⁺

Synthesis of trans-tert-butyl 4-aminocyclohexane-1-carboxylate. To a solution of trans-tert-butyl 4-(((benzyloxy)carbonyl)amino)cyclohexane-1-carboxylate (90.0 g, 270 mmol, 1.00 eq) in THE (1800 mL) was added 10% Pd/C (180 g, 50.0% purity) at 20 °C under N₂. The mixture was degassed and purged with H₂ for 3 times and the resulting mixture was stirred at 20 °C for 12 h under H₂ (15 psi). LCMS showed trans-tert-butyl 4-(((benzyloxy)carbonyl)amino)cyclohexane-1-carboxylate was consumed and the desired target MS was detected. The mixture was filtered and the filtrate was concentrated under reduced pressure to give the title compound (50.0 g, crude) as a brown oil. LCMS (ES⁺): m/z 200.2 [M+H]⁺

Synthesis of 1-ethyl-1-methyl-4-oxopiperidin-1-ium iodide. To a solution of 1-ethylpiperidin-4-one (50.0 g, 393 mmol, 51.8 mL, 1.00 eq) in ACETONE (1000 mL) was added Mel (83.7 g, 590 mmol, 36.7 mL, 1.50 eq) at 0 °C. The mixture was stirred at 0 °C for 0.5 h. Then the mixture was slowly warmed to 20 °C and stirred at 20 °C for 12 h. LCMS showed 1-ethylpiperidin-4-one remained and the desired target MS was detected. Then Mel (11.2 g, 78.6 mmol, 4.89 mL, 0.200 eq) was added to the mixture at 0 °C, The mixture was stirred at 0 °C for 0.5 h. The mixture was slowly warmed to 20 °C and stirred at 20 °C for another 12 h. LCMS showed 1-ethylpiperidin-4-one was consumed completely and the desired target MS was detected. The mixture was filtered and the filter cake was dried under reduced pressure to give the title compound (104 g, crude) as a white solid. LCMS (ES⁺): m/z 142.2 [M−I⁻]⁺

Synthesis of trans-tert-butyl 4-(4-oxopiperidin-1-yl)cyclohexane-1-carboxylate. To a solution of 1-ethyl-1-methyl-4-oxopiperidin-1-ium iodide (28.3 g, 105 mmol, 1.00 eq) and trans-tert-butyl 4-aminocyclohexane-1-carboxylate (21.0 g, 105 mmol, 1.00 eq) in EtOH (500 mL) and H₂O (300 mL) was added K₂CO₃ (43.7 g, 316 mmol, 3.00 eq) at 20 °C. The mixture was stirred at 80 °C for 12 h. LCMS showed 1-ethyl-1-methyl-4-oxopiperidin-1-ium iodide was consumed and the desired target MS was detected. The mixture was concentrated under reduced pressure to remove EtOH. Then the remaining aqueous phase was extracted with EtOAc (500 mL*3), the combined organic layers were dried over Na₂SO₄, filtered and the filtrate was concentrated under reduced pressure to give a residue. The residue was purified by MPLC (SiO₂, PE/ETOAC=1/0 to 0/1) to give the title compound (16.0 g, 56.9 mmol, 53.9% yield) as a yellow oil. LCMS (ES⁺): m/z 282.3 [M+H]⁺

Synthesis of trans-tert-butyl 4-(4-(((trifluoromethyl)sulfonyl)oxy)-3,6-dihydropyridin-1(2H)-yl)cyclohexane-1-carboxylate. To a solution of trans-tert-butyl4-(4-oxopiperidin-1-yl)cyclohexane-1-carboxylate (15.0 g, 53.3 mmol, 1.00 eq) in THF (200 mL) was added LiHMDS (1.00 M, 160 mL, 3.00 eq) at −78 °C under N₂ atmosphere. The mixture was stirred at −78 °C for 1 h under N₂ atmosphere. Then a solution of 1,1,1-trifluoro-N-phenyl-N-(trifluoromethylsulfonyl)methanesulfonamide (28.6 g, 80.0 mmol, 1.50 eq) in THF (200 mL) was added to the mixture at −78 °C. The mixture was slowly warmed to 20 °C and stirred at 20 °C for 12 h. LCMS showed trans-tert-butyl4-(4-oxopiperidin-1-yl)cyclohexane-1-carboxylate was consumed and the desired target MS was detected. Saturated NH₄Cl aqueous solution (600 mL) was added to the mixture dropwise to quench the reaction at 0 °C. Then the reaction mixture was extracted with EtOAc (300 mL*3). The combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (13.5 g, 32.7 mmol, 61.3% yield) as a yellow solid. LCMS (ES⁺): m/z 414.2 [M+H]⁺

Synthesis of trans-tert-butyl 4-(4-(2-fluoro-4-nitrophenyl)-3,6-dihydropyridin-1(2H)-yl)cyclohexane-1-carboxylate. To a solution of 2-(2-fluoro-4-nitrophenyl)-4,4,5,5-tetramethyl-1,3,2-dioxaborolane (6.46 g, 24.2 mmol, 1.00 eq) and trans-tert-butyl4-(4-(((trifluoromethyl)sulfonyl)oxy)-3,6-dihydropyridin-1(2H)-yl)cyclohexane-1-carboxylate (10.0 g, 24.2 mmol, 1.00 eq) in dioxane (210 mL) and H₂O (70 mL) were added Pd(PPh₃)₄ (559 mg, 484 μmol, 0.0200 eq) and Na₂CO₃ (5.13 g, 48.4 mmol, 2.00 eq) under N₂ atmosphere at 20 °C. The mixture was stirred at 90 °C for 4 h under N₂ atmosphere. LCMS showed 2-(2-fluoro-4-nitrophenyl)-4,4,5,5-tetramethyl-1,3,2-dioxaborolane was consumed and the desired target MS was detected. H₂O (200 mL) was added to the mixture and the mixture was extracted with EtOAc (200 mL*3). The combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (7.00 g, 17.3 mmol, 71.6% yield) as a yellow solid. LCMS (ES⁺): m/z 405.3 [M+H]⁺

Synthesis of trans-tert-butyl4-(4-(4-amino-2-fluorophenyl)piperidin-1-yl)cyclohexane-1-carboxylate. To a solution of trans-tert-butyl4-(4-(2-fluoro-4-nitrophenyl)-3,6-dihydropyridin-1(2H)-yl)cyclohexane-1-carboxylate (7.00 g, 17.3 mmol, 1.00 eq) in THF (70 mL) was added 10% Pd/C (3.00 g, 50.0% purity) at 20 °C under N₂. The mixture was degassed and purged with H₂ for 3 times and the mixture was stirred at 20 °C for 4 h under H₂ (15 psi). LCMS showed trans-tert-butyl4-(4-(2-fluoro-4-nitrophenyl)-3,6-dihydropyridin-1(2H)-yl)cyclohexane-1-carboxylate was consumed and the desired target MS was detected. The mixture was filtered, and the filtrate was concentrated under reduced pressure to give the title compound (6.00 g, crude) as a yellow solid. LCMS (ES⁺): m/z 377.2 [M+H]⁺

Synthesis of trans-tert-butyl4-(4-(4-((2,6-dioxopiperidin-3-yl)amino)-2-fluorophenyl)piperidin-1-yl)cyclohexane-1-carboxylate. To a solution of trans-tert-butyl4-(4-(4-amino-2-fluorophenyl)piperidin-1-yl)cyclohexane-1-carboxylate (6.00 g, 15.9 mmol, 1.00 eq) and 3-bromopiperidine-2,6-dione (9.18 g, 47.8 mmol, 3.00 eq) in DMF (100 mL) was added DIEA (6.18 g, 47.8 mmol, 8.33 mL, 3.00 eq) at 20 °C. The mixture was stirred at 80 °C for 12 h. LCMS showed trans-tert-butyl4-(4-(4-amino-2-fluorophenyl)piperidin-1-yl)cyclohexane-1-carboxylate remained and the desired target MS was detected. Then 3-bromopiperidine-2,6-dione (4.59 g, 23.9 mmol, 1.50 eq) and DIEA (2.06 g, 15.9 mmol, 2.78 mL, 1.00 eq) were added to the mixture at 20 °C. The mixture was stirred at 80 °C for another 12 h. LCMS showed trans-tert-butyl4-(4-(4-amino-2-fluorophenyl)piperidin-1-yl)cyclohexane-1-carboxylate was consumed completely and the desired target MS was detected. H₂O (200 mL) was added to the solution, the mixture was extracted with EtOAc (100 mL*3), the combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (2.90 g, 5.95 mmol, 37.3% yield) as a green oil. LCMS (ES⁺): m/z 488.3 [M+H]⁺

Synthesis of trans-4-(4-(4-((2,6-dioxopiperidin-3-yl)amino)-2-fluorophenyl)piperidin-1-yl)cyclohexane-1-carboxylic acid. A solution of trans-tert-butyl4-(4-(4-((2,6-dioxopiperidin-3-yl)amino)-2-fluorophenyl)piperidin-1-yl)cyclohexane-1-carboxylate (500 mg, 1.03 mmol, 1.00 eq) in DCM (5 mL) and TFA (1 mL) was stirred at 20 °C for 1.5 h. LCMS showed trans-tert-butyl4-(4-(4-((2,6-dioxopiperidin-3-yl)amino)-2-fluorophenyl)piperidin-1-yl)cyclohexane-1-carboxylate was consumed and the desired target MS was detected. The mixture was concentrated under reduced pressure to give the title compound (442 mg, crude) as a yellow oil. LCMS (ES⁺): m/z 432.2 [M+H]⁺

Synthesis of rac-(1r,4R)-4-(4-(4-((2,6-dioxopiperidin-3-yl)amino)-2-fluorophenyl)piperidin-1-yl)-N-((1r,4R)-4-((5-fluoro-4-(3-(2-oxopyridin-1(2H)-yl)phenyl)pyrimidin-2-yl)amino)cyclohexyl)cyclohexane-1-carboxamide (Compound 345). To a solution of trans-4-(4-(4-((2,6-dioxopiperidin-3-yl)amino)-2-fluorophenyl)piperidin-1-yl)cyclohexane-1-carboxylic acid (442 mg, 1.02 mmol, 1.00 eq) in DMF (10 mL) were added HATU (117 mg, 307 μmol, 0.300 eq) and DIEA (662 mg, 5.12 mmol, 891 μL, 5.00 eq) at 20 °C, then trans-1-(3-(2-((4-aminocyclohexyl)amino)-5-fluoropyrimidin-4-yl)phenyl)pyridin-2(1H)-one (202 mg, 410 μmol, 0.400 eq, TFA) was added to the mixture, the mixture was stirred at 20 °C for 1 h. LCMS showed trans-4-(4-(4-((2,6-dioxopiperidin-3-yl)amino)-2-fluorophenyl)piperidin-1-yl)cyclohexane-1-carboxylic acid was consumed and the desired target MS was detected. The mixture was concentrated under reduced pressure to give a residue. The residue was purified by prep-HPLC (column: 3_Phenomenex Luna C18 75*30 mm*3 um; mobile phase: [H₂O (0.1% TFA)-ACN]; gradient: 10%-40% B over 8.0 min) to give the title compound (Compound 345) (170 mg, 211 μmol, 20.6% yield, 98.5% purity) as a yellow solid. LCMS (ES⁺): m/z 793.4 [M+H]⁺

Synthesis of (1R,4R)-4-(4-(4-(((R)-2,6-dioxopiperidin-3-yl)amino)-2-fluorophenyl)piperidin-1-yl)-N-((1r,4R)-4-((5-fluoro-4-(3-(2-oxopyridin-1(2H)-yl)phenyl)pyrimidin-2-yl)amino)cyclohexyl)cyclohexane-1-carboxamide (Compound 706) and (1S,4R)-4-(4-(4-(((S)-2,6-dioxopiperidin-3-yl)amino)-2-fluorophenyl)piperidin-1-yl)-N-((1r,4R)-4-((5-fluoro-4-(3-(2-oxopyridin-1(2H)-yl)phenyl)pyrimidin-2-yl)amino)cyclohexyl)cyclohexane-1-carboxamide (Compound 457). rac-(1r,4R)-4-(4-(4-((2,6-dioxopiperidin-3-yl)amino)-2-fluorophenyl)piperidin-1-yl)-N-((1r,4R)-4-((5-fluoro-4-(3-(2-oxopyridin-1(2H)-yl)phenyl)pyrimidin-2-yl)amino)cyclohexyl)cyclohexane-1-carboxamide (Compound 345) was separated by chiral SFC (column: REGIS (s,s) WHELK-O1 (250 mm*30 mm, 5 um); mobile phase: [CO₂—IPA:ACN=1:1 (0.1% NH₃H₂O)]; B %: 60%, isocratic elution mode) to give the title compound (Compound 706) (28.2 mg, 34.6 μmol, 16.2% yield, 97.3% purity) (retention time: 2.253, peak 1) as a pale yellow solid. LCMS (ES+): m/z 793.4 [M+H]⁺ and the title compound (Compound 457) (15.8 mg, 19.8 μmol, 9.23% yield, 99.3% purity) (retention time: 2.668, peak 2) as a white solid. LCMS (ES+): m/z 793.4 [M+H]⁺

### Example 361. Synthesis of trans-1′-(3-(2,6-dioxopiperidin-3-yl)-2-oxo-2,3-dihydrobenzo[d]oxazol-6-yl)-N-(4-((5-fluoro-4-(3-(2-oxopyridin-1(2H)-yl)phenyl)pyrimidin-2-yl)amino)cyclohexyl)-[1,4′-bipiperidine]-4-carboxamide (Compound 317)

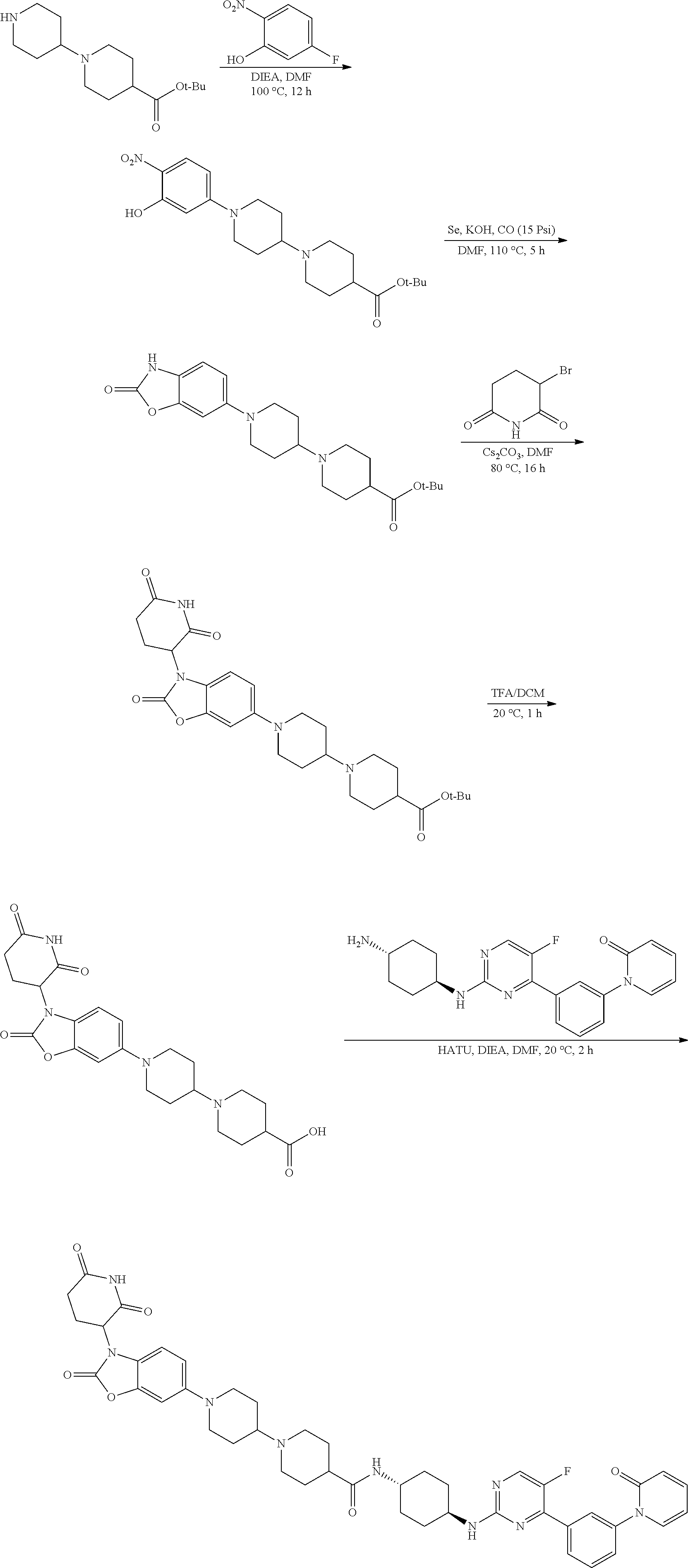

Synthesis of tert-butyl 1′-(3-hydroxy-4-nitrophenyl)-[1,4′-bipiperidine]-4-carboxylate. To a solution of tert-butyl [1,4′-bipiperidine]-4-carboxylate (800 mg, 2.98 mmol, 1.00 eq) and 5-fluoro-2-nitrophenol (468.26 mg, 2.98 mmol, 1.00 eq) in DMF (10 mL) was added DIEA (1.16 g, 8.94 mmol, 1.56 mL, 3.00 eq) at 20 °C. The mixture was stirred at 100 °C for 12 h. LCMS showed tert-butyl [1,4′-bipiperidine]-4-carboxylate was consumed and the desired target MS was detected. H₂O (10 mL) was added to the solution, the mixture was filtered and the filter cake was dried under reduced pressure to give the title compound (950 mg, crude) as a white a solid. LCMS (ES⁺): m/z 406.2 [M+H]⁺

Synthesis of tert-butyl 1′-(2-oxo-2,3-dihydrobenzo[d]oxazol-6-yl)-[1,4′-bipiperidine]-4-carboxylate. To a solution of tert-butyl 1′-(3-hydroxy-4-nitrophenyl)-[1,4′-bipiperidine]-4-carboxylate (900 mg, 2.22 mmol, 1.00 eq) in DMF (10 mL) were added KOH (249 mg, 4.44 mmol, 2.00 eq) and Se (176 mg, 2.22 mmol, 1.00 eq) at 20 °C, the suspension was degassed and purged with CO for three times. The mixture was stirred under CO (15 psi) at 110 °C for 5 h. LCMS showed tert-butyl 1′-(3-hydroxy-4-nitrophenyl)-[1,4′-bipiperidine]-4-carboxylate was consumed and the desired target MS was detected. H₂O (10 mL) was added to the mixture and the mixture was extracted with EtOAc (10 mL*3), the combined organic layers were washed with brine (10 mL), dried over Na₂SO₄, filtered and the filtrate was concentrated under reduced pressure to give the title compound (530 mg, crude) as a red a solid. LCMS (ES⁺): m/z 402.3 [M+H]⁺

Synthesis of tert-butyl 1′-(3-(2,6-dioxopiperidin-3-yl)-2-oxo-2,3-dihydrobenzo[d]oxazol-6-yl)-[1,4′-bipiperidine]-4-carboxylate. To a solution of tert-butyl 1-(2-oxo-2,3-dihydrobenzo[d]oxazol-6-yl)-[1,4′-bipiperidine]-4-carboxylate (430 mg, 1.07 mmol, 1.00 eq) and 3-bromopiperidine-2,6-dione (411 mg, 2.14 mmol, 2.00 eq) in DMF (10 mL) was added Cs₂CO₃ (698 mg, 2.14 mmol, 2.00 eq) at 20 °C, then the mixture was stirred at 80 °C for 12 h. LCMS showed tert-butyl 1′-(2-oxo-2,3-dihydrobenzo[d]oxazol-6-yl)-[1,4′-bipiperidine]-4-carboxylate remained and the desired target MS was detected. Then 3-bromopiperidine-2,6-dione (206 mg, 1.07 mmol, 1.00 eq) and Cs₂CO₃ (349 mg, 1.07 mmol, 1.00 eq) were added to the mixture at 20 °C, the mixture was stirred at 80 °C for another 4 h. LCMS showed tert-butyl 1′-(2-oxo-2,3-dihydrobenzo[d]oxazol-6-yl)-[1,4′-bipiperidine]-4-carboxylate was consumed completely and the desired target MS was detected. H₂O (10 mL) was added to the mixture and the mixture was extracted with EtOAc (10 mL*3), the combined organic layers were washed with brine (10 mL), dried over Na₂SO₄, filtered and the filtrate was concentrated under reduced pressure to give the residue. The residue was purified by prep-TLC (SiO₂, PE/THF=0/1) to give the title compound (200 mg, 390 μmol, 36.4% yield) as a yellow oil. LCMS (ES⁺): m/z 513.3[M+H]⁺

Synthesis of 1′-(3-(2,6-dioxopiperidin-3-yl)-2-oxo-2,3-dihydrobenzo[d]oxazol-6-yl)-[1,4′-bipiperidine]-4-carboxylic acid. A solution of tert-butyl 1′-(3-(2,6-dioxopiperidin-3-yl)-2-oxo-2,3-dihydrobenzo[d]oxazol-6-yl)-[1,4′-bipiperidine]-4-carboxylate (160 mg, 312 mol, 1.00 eq) in DCM (2 mL) and TFA (0.4 mL) was stirred at 20 °C for 40 min. LCMS showed tert-butyl 1′-(3-(2,6-dioxopiperidin-3-yl)-2-oxo-2,3-dihydrobenzo[d]oxazol-6-yl)-[1,4′-bipiperidine]-4-carboxylate remained and the desired target MS was detected. Then the mixture was stirred at 20 °C for another 20 min. LCMS showed tert-butyl 1′-(3-(2,6-dioxopiperidin-3-yl)-2-oxo-2,3-dihydrobenzo[d]oxazol-6-yl)-[1,4′-bipiperidine]-4-carboxylate was consumed completely and the desired target MS was detected. The mixture was concentrated under reduced pressure to give the title compound (140 mg, crude) as a yellow oil. LCMS (ES⁺): m/z 457.2 [M+H]⁺

Synthesis of trans-1′-(3-(2,6-dioxopiperidin-3-yl)-2-oxo-2,3-dihydrobenzo[d]oxazol-6-yl)-N-(4-((5-fluoro-4-(3-(2-oxopyridin-1(2H)-yl)phenyl)pyrimidin-2-yl)amino)cyclohexyl)-[1,4′-bipiperidine]-4-carboxamide (Compound 317). To a solution of 1′-(3-(2,6-dioxopiperidin-3-yl)-2-oxo-2,3-dihydrobenzo[d]oxazol-6-yl)-[1,4′-bipiperidine]-4-carboxylic acid (100 mg, 219 μmol, 1.00 eq) in DMF (2 mL) were added HATU (83.3 mg, 219 μmol, 1.00 eq) and DIEA (142 mg, 1.10 mmol, 190 μL, 5.00 eq) at 20 °C, then trans-1-(3-(2-((4-aminocyclohexyl)amino)-5-fluoropyrimidin-4-yl)phenyl)pyridin-2(1H)-one (63.8 mg, 153 μmol, 0.700 eq, HCl) was added to the mixture, the mixture was stirred at 20 °C for 2 h. LCMS showed 1′-(3-(2,6-dioxopiperidin-3-yl)-2-oxo-2,3-dihydrobenzo[d]oxazol-6-yl)-[1,4′-bipiperidine]-4-carboxylic acid was consumed and the desired target MS was detected. The mixture was concentrated under reduced pressure to give a residue. The residue was purified by prep-HPLC (column: Phenomenex Luna C18 75*30 mm*3 um; mobile phase: [H₂O (0.1% TFA)-ACN]; gradient: 15%-40% B over 8.0 min) to give the title compound (Compound 317) (20.5 mg, 24.5 μmol, 11.2% yield, 97.8% purity) as a gray solid. LCMS (ES⁺): m/z 818.4[M+H]⁺

### Example 362. Synthesis of trans-2-(1-(4-((2,6-dioxopiperidin-3-yl)amino)-2-fluorophenyl)piperidin-4-yl)-2-azaspiro[3.3]heptan-6-yl (4-((5-fluoro-4-(3-(2-oxopyridin-1(2H)-yl)phenyl)pyrimidin-2-yl)amino)cyclohexyl)carbamate (Compound 377)

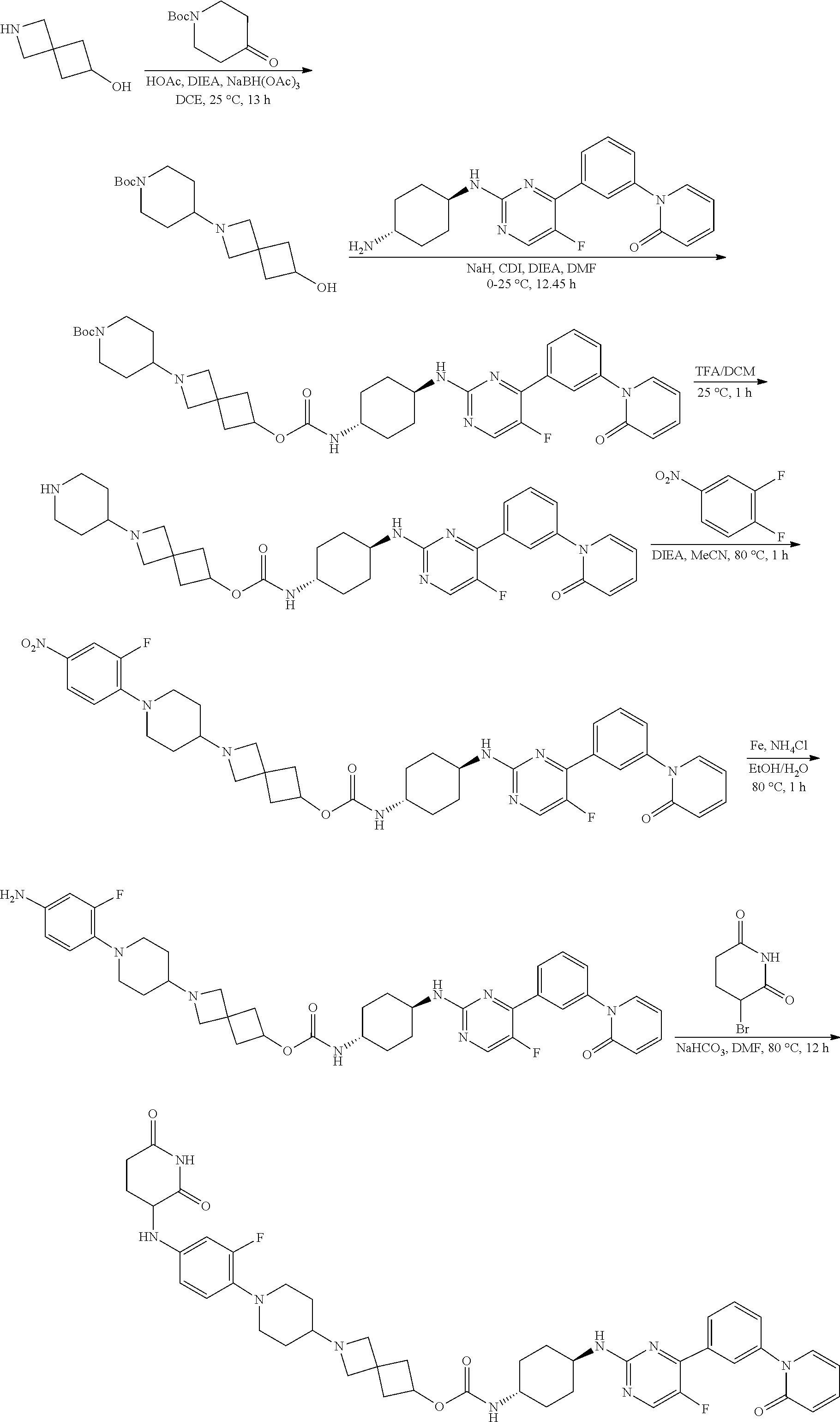

Synthesis of tert-butyl 4-(6-hydroxy-2-azaspiro[3.3]heptan-2-yl)piperidine-1-carboxylate. To a solution of 2-azaspiro[3.3]heptan-6-ol (2.50 g, 22.0 mmol, 1.00 eq) in DCE (30 mL) was added DIEA (8.57 g, 66.2 mmol, 11.5 mL, 3.00 eq) at 25 °C. The mixture was stirred at 25 °C for 0.5 h. Then HOAc (6.63 g, 110 mmol, 6.32 mL, 5.00 eq) and tert-butyl 4-oxopiperidine-1-carboxylate (13.2 g, 66.2 mmol, 3.00 eq) at 25 °C. The mixture was stirred at 25 °C for 0.5 h. Then NaBH(OAc)₃ (14.0 g, 66.2 mmol, 3.00 eq) was added at 25 °C. The mixture was stirred at 25 °C for 12 h. LC-MS showed the desired mass was detected. The reaction mixture was diluted with water 20 mL and extracted with DCM (30 mL*3), filtered and concentrated under reduced pressure. The residue was purified by column chromatography (SiO₂, Dichloromethane:Methanol=1:0 to 10:1) to give the title compound (3.54 g, 7.17 mmol, 32.4% yield, 60.0% purity) as a yellow oil. LCMS (ES⁺): m/z 297.2 [M+H]⁺.

Synthesis of tert-butyl 4-(6-(((trans-4-((5-fluoro-4-(3-(2-oxopyridin-1(2H)-yl)phenyl)pyrimidin-2-yl)amino)cyclohexyl)carbamoyl)oxy)-2-azaspiro[3.3]heptan-2-yl)piperidine-1-carboxylate. To a solution of trans-1-(3-(2-((4-aminocyclohexyl)amino)-5-fluoropyrimidin-4-yl)phenyl)pyridin-2(1H)-one (1.50 g, 3.61 mmol, 1.00 eq, HCl) in DMF (8 mL) was added DIEA (932 mg, 7.21 mmol, 1.26 mL, 2.00 eq) and CDI (584 mg, 3.61 mmol, 1.00 eq) at 25 °C. The mixture was stirred at 25 °C for 0.5 h. Then tert-butyl 4-(6-hydroxy-2-azaspiro[3.3]heptan-2-yl)piperidine-1-carboxylate (1.07 g, 3.61 mmol, 1.00 eq) in DMF (7 mL) was added NaH (288 mg, 7.21 mmol, 60.0% purity, 2.00 eq) was added at 0 °C for 15 min. Then two of mixture was stirred at 25 °C for 12 h. LC-MS showed the desired mass was detected. The reaction mixture was diluted with water 7 mL and extracted with EtOAc (15 mL*3). The combined organic layers dried over Na₂SO₄, filtered and concentrated under reduced pressure. The residue was purified by column chromatography (SiO₂, Dichloromethane:Methanol=1:0 to 10:1) to give the title compound (1.50 g, 1.69 mmol, 46.9% yield, 79.2% purity) as a yellow solid. LCMS (ES⁺): m/z 702.4 [M+H]⁺.

Synthesis of trans-2-(piperidin-4-yl)-2-azaspiro[3.3]heptan-6-yl (4-((5-fluoro-4-(3-(2-oxopyridin-1(2H)-yl)phenyl)pyrimidin-2-yl)amino)cyclohexyl)carbamate. To a solution of trans-tert-butyl 4-(6-((4-((5-fluoro-4-(3-(2-oxopyridin-1(2H)-yl)phenyl)pyrimidin-2-yl)amino)cyclohexyl)carbamoyl)oxy)-2-azaspiro[3.3]heptan-2-yl)piperidine-1-carboxylate (600 mg, 854 μmol, 1.00 eq) in DCM (4 mL) was added TFA (3.07 g, 26.9 mmol, 2 mL, 31.4 eq) at 25 °C. The mixture was stirred at 25 °C for 1 h. trans-tert-butyl 4-(6-((4-((5-fluoro-4-(3-(2-oxopyridin-1(2H)-yl)phenyl)pyrimidin-2-yl)amino)cyclohexyl)carbamoyl)oxy)-2-azaspiro[3.3]heptan-2-yl)piperidine-1-carboxylate was consumed completely and one new spot formed. The reaction was clean according to TLC. The reaction mixture was concentrated under reduced pressure to give the title compound (1.30 g, crude, TFA) as a yellow oil. LCMS (ES⁺): m/z 602.4 [M+H]⁺.

Synthesis of trans-2-(1-(2-fluoro-4-nitrophenyl)piperidin-4-yl)-2-azaspiro[3.3]heptan-6-yl (4-((5-fluoro-4-(3-(2-oxopyridin-1(2H)-yl)phenyl)pyrimidin-2-yl)amino)cyclohexyl)carbamate. To a solution of trans-2-(piperidin-4-yl)-2-azaspiro[3.3]heptan-6-yl (4-((5-fluoro-4-(3-(2-oxopyridin-1(2H)-yl)phenyl)pyrimidin-2-yl)amino)cyclohexyl)carbamate (520 mg, 726 μmol, 1.00 eq, TFA) in MeCN (5 mL) was added 1,2-difluoro-4-nitro-benzene (115 mg, 726 μmol, 80.4 μL, 1.00 eq), DIEA (281 mg, 2.18 mmol, 379 μL, 3.00 eq) at 25 °C. The mixture was stirred at 80 °C for 1 h. LC-MS showed the desired mass was detected. The reaction mixture was concentrated under reduced pressure to give a residue. The residue was purified by column chromatography (SiO₂, Dichloromethane:Methanol=1:0 to 10:1) to give the title compound (1.55 g, crude) as a yellow oil. LCMS (ES⁺): m/z 741.4 [M+H]⁺.

Synthesis of trans-2-(1-(4-amino-2-fluorophenyl)piperidin-4-yl)-2-azaspiro[3.3]heptan-6-yl (4-((5-fluoro-4-(3-(2-oxopyridin-1(2H)-yl)phenyl)pyrimidin-2-yl)amino)cyclohexyl)carbamate. To a solution of trans-2-(1-(2-fluoro-4-nitrophenyl)piperidin-4-yl)-2-azaspiro[3.3]heptan-6-yl (4-((5-fluoro-4-(3-(2-oxopyridin-1(2H)-yl)phenyl)pyrimidin-2-yl)amino)cyclohexyl)carbamate (100 mg, 134 μmol, 1.00 eq) in EtOH (5 mL) and H₂O (1 mL) was added Fe (37.6 mg, 674 μmol, 5.00 eq) and NH₄Cl (36.1 mg, 674 μmol, 5.00 eq) at 25 °C. The mixture was stirred at 80 °C for 1 h. TLC indicated trans-2-(1-(2-fluoro-4-nitrophenyl)piperidin-4-yl)-2-azaspiro[3.3]heptan-6-yl (4-((5-fluoro-4-(3-(2-oxopyridin-1(2H)-yl)phenyl)pyrimidin-2-yl)amino)cyclohexyl)carbamate was consumed completely and one new spot formed. The reaction was clean according to TLC. The reaction mixture was filtered and the filtrate concentrated under high vacuum to give the title compound (100 mg, crude) as a brown solid. LCMS (ES⁺): m/z 711.4 [M+H]⁺.

Synthesis of trans-2-(1-(4-((2,6-dioxopiperidin-3-yl)amino)-2-fluorophenyl)piperidin-4-yl)-2-azaspiro[3.3]heptan-6-yl (4-((5-fluoro-4-(3-(2-oxopyridin-1(2H)-yl)phenyl)pyrimidin-2-yl)amino)cyclohexyl)carbamate (Compound 377). To a solution of trans-2-(1-(4-amino-2-fluorophenyl)piperidin-4-yl)-2-azaspiro[3.3]heptan-6-yl (4-((5-fluoro-4-(3-(2-oxopyridin-1(2H)-yl)phenyl)pyrimidin-2-yl)amino)cyclohexyl)carbamate (100 mg, 140 μmol, 1.00 eq) in DMF (4 mL) was added NaHCO₃ (47.2 mg, 562 μmol, 21.9 μL, 4.00 eq) and 3-bromopiperidine-2,6-dione (135 mg, 703 μmol, 5.00 eq) at 25 °C. The mixture was stirred at 80 °C for 12 h. LC-MS showed the desired mass was detected. The reaction mixture was filtered to give a residue. The residue was purified by prep-HPLC (column: Phenomenex luna C18 100*40 mm*5 um; mobile phase: [H₂O (0.2% FA)-ACN]; gradient: 15%-55% B over 8.0 min) to give the title compound (Compound 377) (6.10 mg, 7.07 μmol, 5.02% yield, 95.2% purity) as a brown solid. LCMS (ES⁺): m/z 822.4 [M+H]⁺.

### Example 363. Synthesis of trans-1′-(4-((2,6-dioxopiperidin-3-yl)amino)-2-fluorophenyl)-3′,3′-difluoro-N-(4-((5-fluoro-4-(3-(2-oxopyridin-1(2H)-yl)phenyl)pyrimidin-2-yl)amino)cyclohexyl)-[1,4′-bipiperidine]-4-carboxamide (Compound 598)

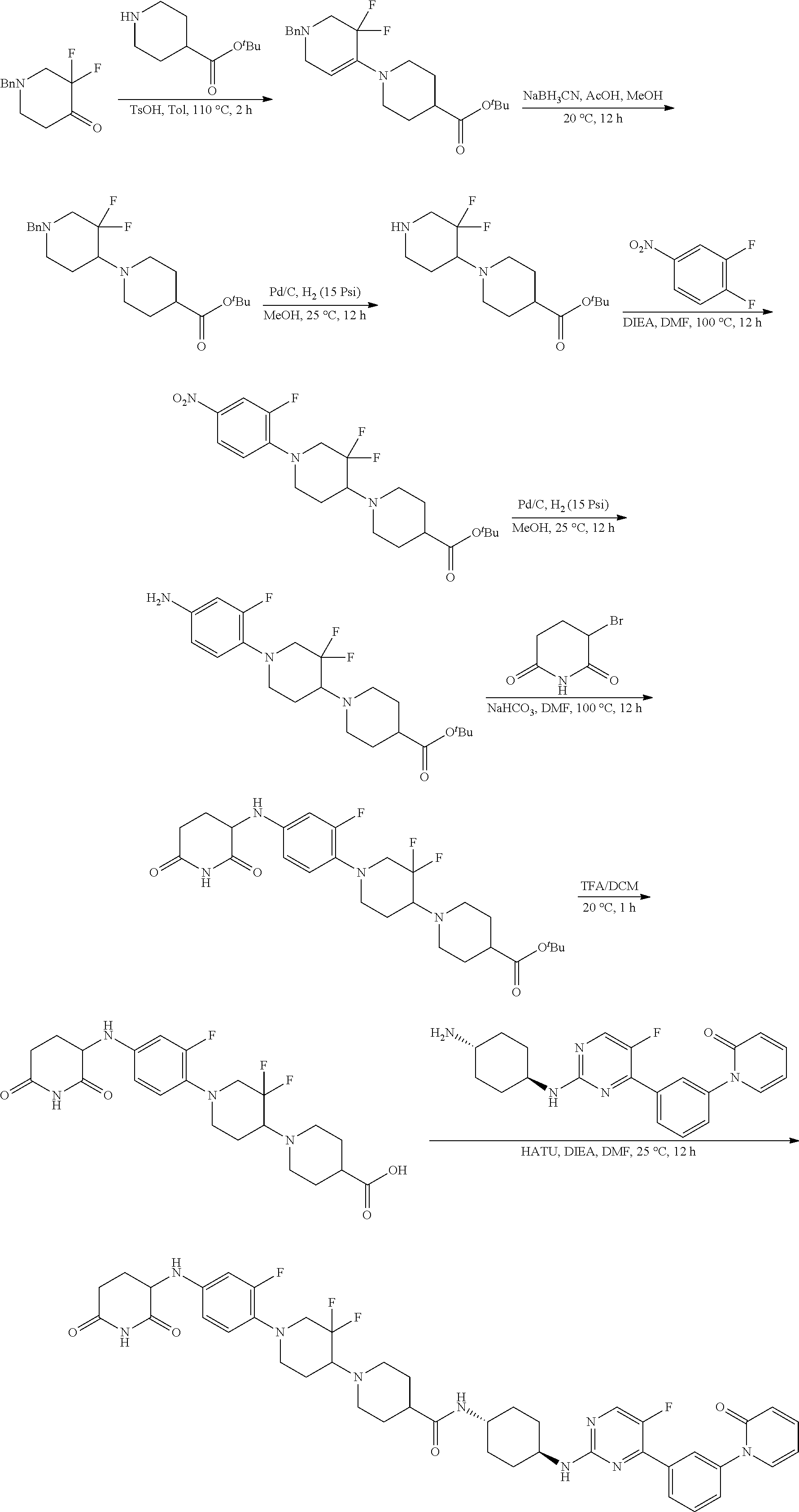

Synthesis of tert-butyl 1-(1-benzyl-3,3-difluoro-1,2,3,6-tetrahydropyridin-4-yl)piperidine-4-carboxylate. To a solution of 1-benzyl-3,3-difluoropiperidin-4-one (10.0 g, 44.4 mmol, 1.00 eq) in Tol. (30 mL) was added tert-butyl piperidine-4-carboxylate (20.5 g, 111 mmol, 2.50 eq), TsOH (764 mg, 4.44 mmol, 0.100 eq) at 20 °C and remove water by Dean-Stark trap. The mixture was stirred at 110 °C for 2 h. LC-MS showed the desired mass was detected. The reaction mixture was concentrated under high vacuum to give the title compound (21 g, crude) as a white solid. LCMS (ES⁺): m/z 393.3 [M+H]⁺.

Synthesis of tert-butyl 1′-benzyl-3′,3′-difluoro-[1,4′-bipiperidine]-4-carboxylate. To a solution of tert-butyl 1-(1-benzyl-3,3-difluoro-1,2,3,6-tetrahydropyridin-4-yl)piperidine-4-carboxylate (10.0 g, 25.4 mmol, 1.00 eq) in MeOH (100 mL) was added dropwise AcOH (3.05 g, 50.8 mmol, 2.91 mL, 2.00 eq) at 20 °C over 0.5 h. After addition, the mixture was stirred at this temperature for 0.5 h, and then NaBH₃CN (3.19 g, 50.8 mmol, 2.00 eq) was added portion wise at 20 °C. The resulting mixture was stirred at 20 °C for 11 h. LC-MS showed the desired mass was detected. The reaction mixture was quenched by addition H₂O 10 mL, and then diluted with H₂O 10 mL and extracted with EtOAc (10 mL*3). The combined organic layers were washed with sat.aq. NaCl (10 mL), dried over Na₂SO₄, filtered and concentrated under reduced pressure. The residue was purified by column chromatography (SiO₂, Petroleum ether:Ethyl acetate=1:0 to 1:1) to give the title compound (21 g, crude) as a colourless oil. LCMS (ES⁺): m/z 395.2 [M+H]⁺.

Synthesis of tert-butyl 3′,3′-difluoro-[1,4′-bipiperidine]-4-carboxylate. To a solution of tert-butyl 1′-benzyl-3′,3′-difluoro-[1,4′-bipiperidine]-4-carboxylate (15.0 g, 38.0 mmol, 1.00 eq) in MeOH (150 mL) was added 10% Pd/C (4.00 g, 50% purity) at 0 °C under N₂ atmosphere. The suspension was degassed and purged with H₂ for 3 times. The mixture was stirred under H₂ (15 Psi) at 25 °C for 12 h. TLC (Petroleum ether:Ethyl acetate=0:1, Rf=0.1) indicated tert-butyl 1′-benzyl-3′,3′-difluoro-[1,4′-bipiperidine]-4-carboxylate was consumed completely and one main new spot formed. The reaction mixture was filtered and the filtrate concentrated under high vacuum to give the title compound as a white solid. LCMS (ES⁺): m/z 305.2 [M+H]⁺.

Synthesis of tert-butyl 3′,3′-difluoro-1′-(2-fluoro-4-nitrophenyl)-[1,4′-bipiperidine]-4-carboxylate. To a solution of tert-butyl 3′,3′-difluoro-[1,4′-bipiperidine]-4-carboxylate (5.50 g, 18.0 mmol, 1.00 eq) in DMF (30 mL) was added 1,2-difluoro-4-nitro-benzene (4.31 g, 27.1 mmol, 3.00 mL, 1.50 eq), DIEA (4.67 g, 36.1 mmol, 6.29 mL, 2.00 eq) at 20 °C. The mixture was stirred at 100 °C for 12 h. LC-MS showed the desired mass was detected. The solid was filtered, washed with EtOAc (3*20 mL), and dried under high vacuum to give the title compound (18 g, crude) as a yellow solid. LCMS (ES⁺): m/z 444.2 [M+H]⁺.

Synthesis of tert-butyl 1′-(4-amino-2-fluorophenyl)-3′,3′-difluoro-[1,4′-bipiperidine]-4-carboxylate. To a solution of tert-butyl 3′,3′-difluoro-1′-(2-fluoro-4-nitrophenyl)-[1,4′-bipiperidine]-4-carboxylate (5.00 g, 11.2 mmol, 1.00 eq) in MeOH (100 mL) was added 10% Pd/C (6.00 g, 50% purity) at 0 °C under N₂ atmosphere. The suspension was degassed and purged with H₂ for 3 times. The mixture was stirred under H₂ (15 Psi) at 25 °C for 12 h. LC-MS showed the desired mass was detected. The reaction mixture was filtered and the filtrate concentrated under high vacuum to give the title compound (9.00 g, crude) as a green solid. LCMS (ES⁺): m/z 414.2 [M+H]⁺.

Synthesis of tert-butyl 1′-(4-((2,6-dioxopiperidin-3-yl)amino)-2-fluorophenyl)-3′,3′-difluoro-[1,4′-bipiperidine]-4-carboxylate. A mixture of tert-butyl 1′-(4-amino-2-fluorophenyl)-3′,3′-difluoro-[1,4′-bipiperidine]-4-carboxylate (5.00 g, 12.0 mmol, 1.00 eq), 3-bromopiperidine-2,6-dione (9.29 g, 48.3 mmol, 4.00 eq), NaHCO₃ (3.05 g, 36.2 mmol, 1.41 mL, 3.00 eq) in DMF (20 mL) was degassed and purged with N₂ at 20 °C for 3 times, and then the mixture was stirred at 100 °C for 12 h under N₂ atmosphere. TLC (PE:THF=0:1, Rf=0.41) indicated tert-butyl 1′-(4-amino-2-fluorophenyl)-3′,3′-difluoro-[1,4′-bipiperidine]-4-carboxylate was consumed completely and one main new spot formed. The reaction mixture was quenched by addition H₂O 10 mL, and then diluted with H₂O 20 mL and extracted with EtOAc (30 mL*3). The combined organic layers were washed with sat.aq. NaCl (10 mL), dried over Na₂SO₄, filtered and concentrated under reduced pressure. The residue was purified by column chromatography (SiO₂, PE:THF=1:0 to 0:1) to give the title compound (4.00 g, 6.90 mmol, 57.0% yield, 90.5% purity) as a green solid. LCMS (ES⁺): m/z 525.3 [M+H]⁺.

Synthesis of 1′-(4-((2,6-dioxopiperidin-3-yl)amino)-2-fluorophenyl)-3′,3′-difluoro-[1,4′-bipiperidine]-4-carboxylic acid. To a solution of tert-butyl 1′-(4-((2,6-dioxopiperidin-3-yl)amino)-2-fluorophenyl)-3′,3′-difluoro-[1,4′-bipiperidine]-4-carboxylate (1.00 g, 1.91 mmol, 1 eq) in DCM (5 mL) was added TFA (7.37 g, 64.62 mmol, 4.80 mL, 33.90 eq) at 20 °C. The mixture was stirred at 20 °C for 1 h. TLC (Petroleum ether:Ethyl acetate=0:1, Rf=0.21) indicated tert-butyl 1′-(4-((2,6-dioxopiperidin-3-yl)amino)-2-fluorophenyl)-3′,3′-difluoro-[1,4′-bipiperidine]-4-carboxylate was consumed completely and one main new spot formed. The reaction mixture was concentrated under high vacuum to give the title compound (900 mg, crude, TFA) as a green solid. LCMS (ES⁺): m/z 469.3 [M+H]⁺.

Synthesis of trans-1′-(4-((2,6-dioxopiperidin-3-yl)amino)-2-fluorophenyl)-3′,3′-difluoro-N-(4-((5-fluoro-4-(3-(2-oxopyridin-1(2H)-yl)phenyl)pyrimidin-2-yl)amino)cyclohexyl)-[1,4′-bipiperidine]-4-carboxamide (Compound 598). To a solution of 1′-(4-((2,6-dioxopiperidin-3-yl)amino)-2-fluorophenyl)-3′,3′-difluoro-[1,4′-bipiperidine]-4-carboxylic acid (1.00 g, 2.13 mmol, 1.00 eq) in DMF (1 mL) was added trans-1-(3-(2-((4-aminocyclohexyl)amino)-5-fluoropyrimidin-4-yl)phenyl)pyridin-2(1H)-one (976 mg, 2.35 mmol, 1.10 eq, HCl), HATU (1.62 g, 4.27 mmol, 2.00 eq) and DIEA (551 mg, 4.27 mmol, 743 μL, 2.00 eq). The mixture was stirred at 25 °C for 12 h. LC-MS showed the desired mass was detected. The reaction mixture was quenched by addition H₂O 3 mL, and then extracted with EtOAc (3 mL*3). The combined organic layers were dried over Na₂SO₄, filtered and concentrated under reduced pressure. The residue was purified by prep-HPLC (column: Phenomenex luna C18 100*40 mm*5 um; mobile phase: [H₂O (0.2% FA)-ACN]; gradient: 10%-40% B over 8.0 min) to give the title compound (Compound 598) (371 mg, 425 μmol, 19.9% yield, 95.2% purity) as a brown solid. LCMS (ES⁺): m/z 830.4 [M+H]⁺.

### Example 364. Synthesis of trans-2-(1′-(4-((2,6-dioxopiperidin-3-yl)amino)-2-fluorophenyl)-[!1,4′-bipiperidin]-4-yl)-N-((1r,4r)-4-((5-fluoro-4-(3-(2-oxopyridin-1(2H)-yl)phenyl)pyrimidin-2-yl)amino)cyclohexyl)acetamide (Compound 395)

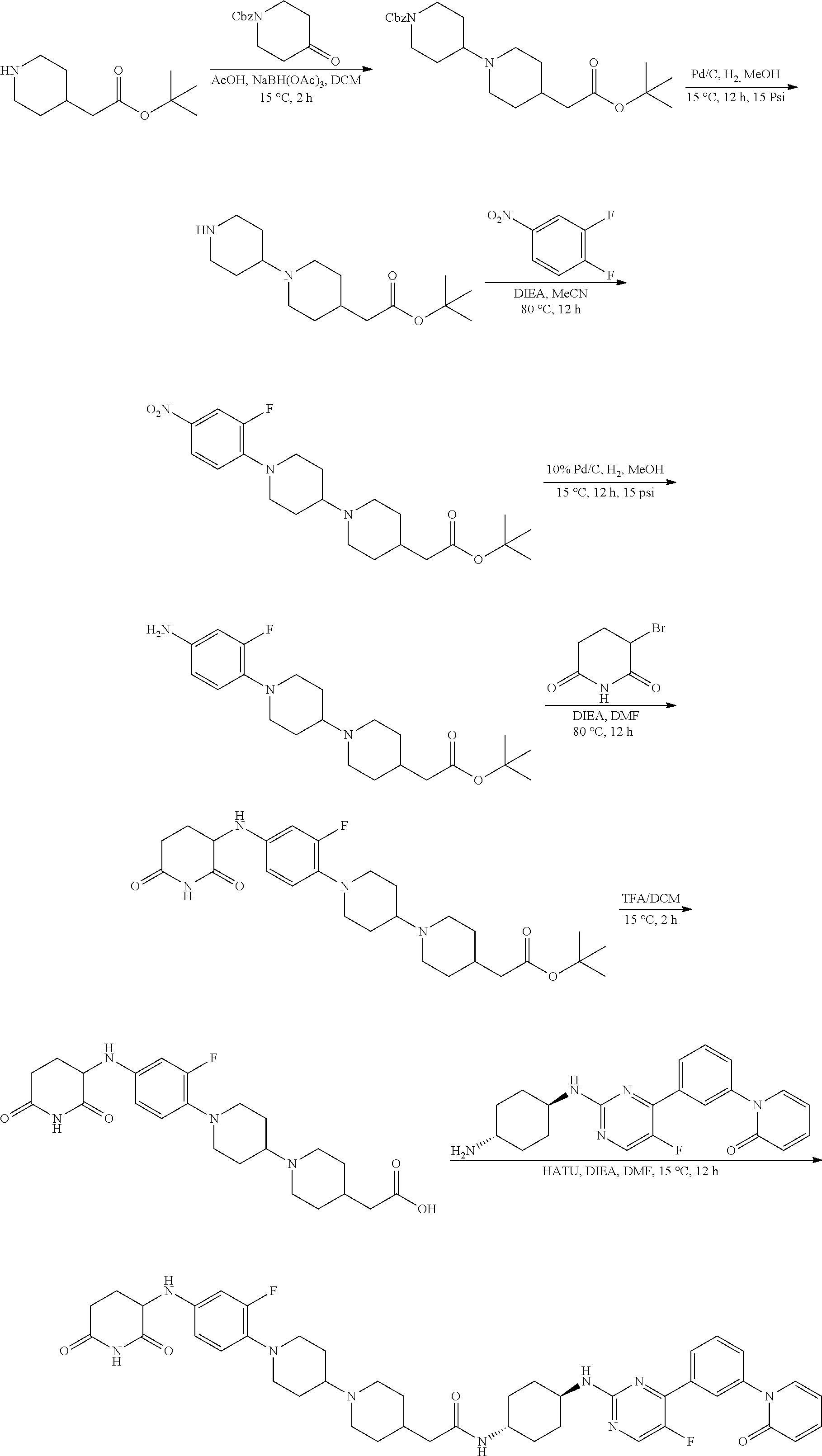

Synthesis of benzyl 4-(2-(tert-butoxy)-2-oxoethyl)-[1,4′-bipiperidine]-1′-carboxylate. To a solution of tert-butyl 2-(piperidin-4-yl)acetate (3.00 g, 15.1 mmol, 1.00 eq) in DCM (100 mL) were added benzyl 4-oxopiperidine-1-carboxylate (4.21 g, 18.1 mmol, 3.60 mL, 1.20 eq), NaBH(OAc)₃ (6.38 g, 30.1 mmol, 2.00 eq) at 15 °C. The mixture was stirred at 15 °C for 2 h. LC-MS showed that tert-butyl 2-(piperidin-4-yl)acetate was consumed completely and the desired mass was detected. The reaction mixture was diluted with water (100 mL) and extracted with DCM (100 mL*3). The combined organic layers were washed with brine (100 mL*3), dried over Na₂SO₄, filtered and the filtrate was concentrated under reduced pressure. The residue was purified by column chromatography (SiO₂, Petroleum ether/Ethyl acetate=3/1 to 0/1) to give title Compound (5.10 g, 12.2 mmol, 81.3% yield) as a yellow oil. LCMS (ES⁺): m/z 417.0 [M+H]⁺.

Synthesis of tert-butyl 2-([1,4′-bipiperidin]-4-yl)acetate. To a mixture of 10% Pd/C (2.50 g, 50% purity) in EtOAc (150 mL) was added benzyl 4-(2-(tert-butoxy)-2-oxoethyl)-[1,4′-bipiperidine]-1′-carboxylate (5.00 g, 12.0 mmol, 1.00 eq) at 15 °C. The mixture was stirred at 15 °C for 12 h under H₂ (15 Psi). LC-MS showed that benzyl 4-(2-(tert-butoxy)-2-oxoethyl)-[1,4′-bipiperidine]-1′-carboxylate was consumed completely and the desired mass was detected. The mixture was concentrated under reduced pressure to give title Compound (3.5 g, crude) as a yellow solid. LCMS (ES⁺): m/z 283.1 [M+H]⁺.

Synthesis of tert-butyl 2-(1′-(2-fluoro-4-nitrophenyl)-[1,4′-bipiperidin]-4-yl)acetate. To a solution of tert-butyl 2-([1,4′-bipiperidin]-4-yl)acetate (3.40 g, 12.0 mmol, 1.00 eq) in ACN (100 mL) were added DIEA (4.67 g, 36.1 mmol, 6.29 mL, 3.00 eq) and 1,2-difluoro-4-nitro-benzene (2.30 g, 14.5 mmol, 1.60 mL, 1.20 eq) at 15 °C. The mixture was stirred at 80 °C for 12 h. LC-MS showed that tert-butyl 2-([1,4′-bipiperidin]-4-yl)acetate was consumed completely and the desired mass was detected. The reaction mixture was concentrated under reduced pressure to give a residue. Water (100 ml) was added and extracted with EtOAc (100 mL*3). The combined organic layers were washed with brine (100 mL*3), dried over Na₂SO₄, filtered and the filtrate was concentrated under reduced pressure. The residue was purified by column chromatography (SiO₂, Petroleum ether/Ethyl acetate=5/1 to 1/1) to give title Compound (3.10 g, 7.35 mmol, 61.09% yield) as a yellow solid. LCMS (ES⁺): m/z 422.2 [M+H]⁺.

Synthesis of tert-butyl 2-(1′-(4-amino-2-fluorophenyl)-[1,4′-bipiperidin]-4-yl)acetate. To a mixture of 10% Pd/C (1.51 g, 50% purity) in MeOH (20 mL) was added tert-butyl 2-(1′-(2-fluoro-4-nitrophenyl)-[1,4′-bipiperidin]-4-yl)acetate (3.00 g, 7.12 mmol, 1.00 eq) at 15 °C. The mixture was stirred at 15 °C for 12 h under H₂ (15 psi). LC-MS showed that tert-butyl 2-(1′-(2-fluoro-4-nitrophenyl)-[1,4′-bipiperidin]-4-yl)acetate was consumed completely and the desired mass was detected. The reaction mixture was filtered and the filtrate was concentrated under reduced pressure to give title Compound (2.30 g, crude) as a yellow solid. LCMS (ES⁺): m/z 392.0 [M+H]⁺.

Synthesis of tert-butyl 2-(1′-(4-((2,6-dioxopiperidin-3-yl)amino)-2-fluorophenyl)-[1,4′-bipiperidin]-4-yl)acetate. To a solution of tert-butyl 2-(1′-(4-amino-2-fluorophenyl)-[1,4′-bipiperidin]-4-yl)acetate (2.20 g, 5.62 mmol, 1.00 eq) in DMF (30 mL) were added NaHCO₃ (944 mg, 11.2 mmol, 437 μL, 2.00 eq) and 3-bromopiperidine-2,6-dione (4.32 g, 22.5 mmol, 4.00 eq) at 15 °C. The mixture was stirred at 80 °C for 12 h. LC-MS showed that tert-butyl 2-(1′-(4-amino-2-fluorophenyl)-[1,4′-bipiperidin]-4-yl)acetate was consumed completely and the desired mass was detected. The reaction mixture was diluted with saturated H₂O (50 mL) and then extracted with EtOAc (100 mL*3). The combined organic layers were washed with brine (100 mL*3), dried over Na₂SO₄, filtered and the filtrate was concentrated under reduced pressure to give title Compound (1.50 g, 2.98 mmol, 53.1% yield) as a yellow solid. LCMS (ES⁺): m/z 503.2 [M+H]⁺.

Synthesis of 2-(1′-(4-((2,6-dioxopiperidin-3-yl)amino)-2-fluorophenyl)-[1,4′-bipiperidin]-4-yl)acetic acid. To a solution of tert-butyl 2-(1′-(4-((2,6-dioxopiperidin-3-yl)amino)-2-fluorophenyl)-[1,4′-bipiperidin]-4-yl)acetate (600 mg, 1.19 mmol, 1.00 eq) in DCM (3 mL) was added TFA (4.61 g, 40.4 mmol, 3.00 mL, 33.8 eq) at 0 °C. The mixture was stirred at 15 °C for 2 h. LC-MS showed that tert-butyl 2-(1′-(4-((2,6-dioxopiperidin-3-yl)amino)-2-fluorophenyl)-[1,4′-bipiperidin]-4-yl)acetate was consumed completely and the desired mass was detected. The mixture was concentrated under reduced pressure to give title Compound (600 mg, crude) as a yellow oil. LCMS (ES⁺): m/z 447.1 [M+H]⁺.

Synthesis of trans-2-(1′-(4-((2,6-dioxopiperidin-3-yl)amino)-2-fluorophenyl)-[1,4′-bipiperidin]-4-yl)-N-((1r,4r)-4-((5-fluoro-4-(3-(2-oxopyridin-1(2H)-yl)phenyl)pyrimidin-2-yl)amino)cyclohexyl)acetamide (Compound 395). To a solution of 2-(1′-(4-((2,6-dioxopiperidin-3-yl)amino)-2-fluorophenyl)-[1,4′-bipiperidin]-4-yl)acetic acid (600 mg, 1.34 mmol, 1.00 eq) in DMF (10 mL) were added DIEA (695 mg, 5.37 mmol, 936 μL, 4.00 eq), trans-1-(3-(2-(((1r,4r)-4-aminocyclohexyl)amino)-5-fluoropyrimidin-4-yl)phenyl)pyridin-2(1H)-one (500 mg, 1.01 mmol, 7.54e-1 eq, TFA) and HATU (766 mg, 2.02 mmol, 1.50 eq) at 15 °C. The mixture was stirred at 15 °C for 12 h. LC-MS showed that 2-(1′-(4-((2,6-dioxopiperidin-3-yl)amino)-2-fluorophenyl)-[1,4′-bipiperidin]-4-yl)acetic acid was consumed completely and the desired mass was detected. The reaction mixture was diluted with water 10 mL and extracted with DCM (10 mL*3). The combined organic layers were washed with brine (10 mL*3), dried over Na₂SO₄, filtered and the filtrate was concentrated under reduced pressure to give a residue. The residue was purified by prep-HPLC (column: Phenomenex luna C18 100*40 mm*5 um; mobile phase: [1H₂O (0.2% FA)-ACN]; gradient: 5%-40% B over 8.0 min) to give title Compound (Compound 395) (257 mg, 307 mol, 22.8% yield, 96.4% purity) as a white solid. LCMS (ES⁺): m/z 808.5 [M+H]⁺.

### Example 365. Synthesis of 1-((1-(4-((2,6-dioxopiperidin-3-yl)amino)-2-fluorophenyl)piperidin-4-yl)methyl)-N-((1r,4r)-4-((5-fluoro-4-(3-(2-oxopyridin-1(2H)-yl)phenyl)pyrimidin-2-yl)amino)cyclohexyl)piperidine-4-carboxamide (Compound 433)

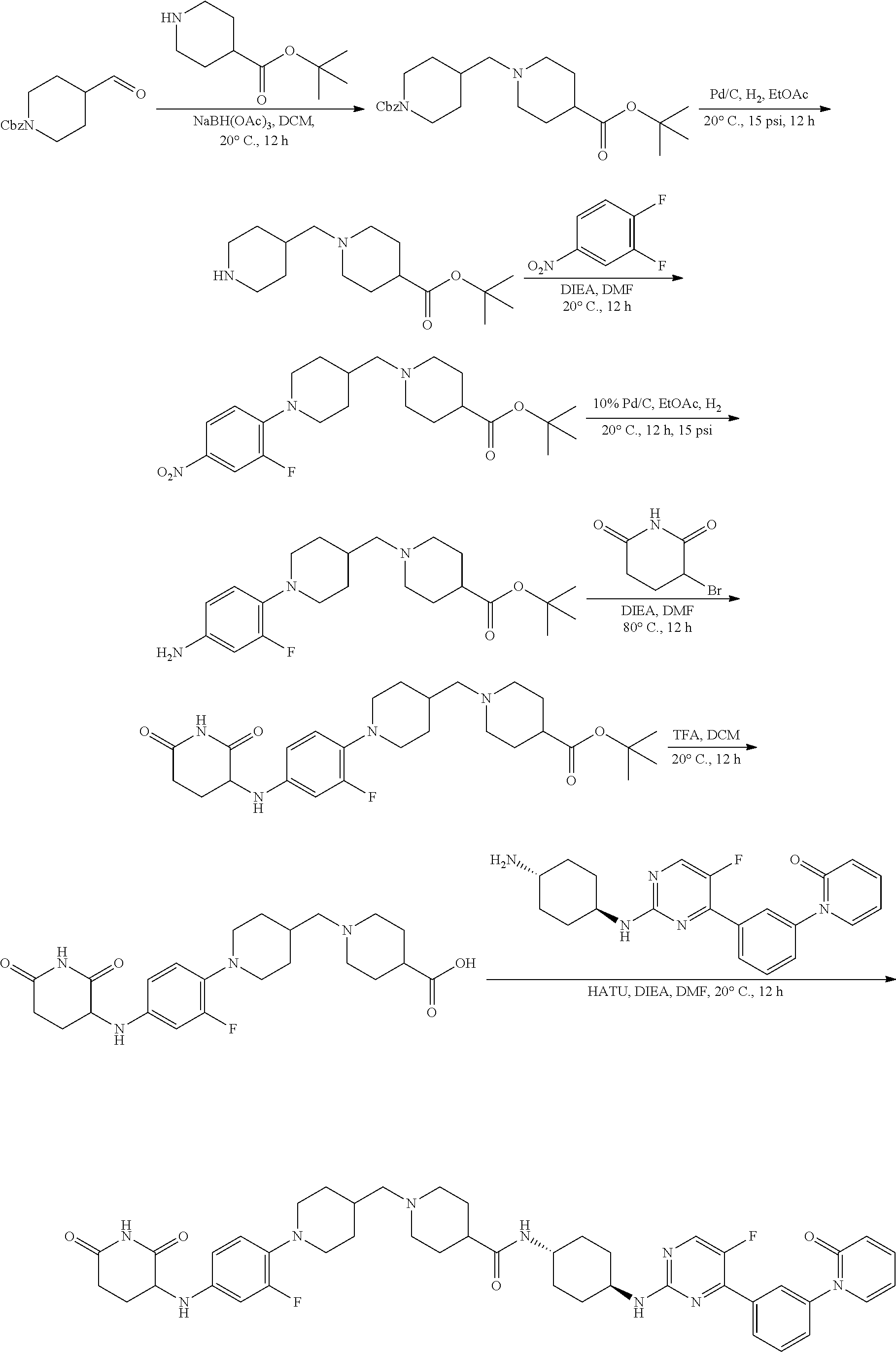

Synthesis of benzyl 4-((4-(tert-butoxycarbonyl)piperidin-1-yl)methyl)piperidine-1-carboxylate. To a solution of benzyl 4-formylpiperidine-1-carboxylate (10.0 g, 40.4 mmol, 1.00 eq) and tert-butyl piperidine-4-carboxylate (15.0 g, 80.9 mmol, 2.00 eq) in DCM (100 mL) was added NaBH(OAc)₃ (17.1 g, 80.9 mmol, 2.00 eq) at 20 °C. The mixture was stirred at 20 °C for 12 h. LC-MS showed benzyl 4-formylpiperidine-1-carboxylate was consumed completely and desired mass was detected. The reaction mixture was concentrated under reduced pressure to remove solvent. The residue was purified by column chromatography (SiO₂, DCM/THF=1/0 to 3/2) to give the title compound (14.0 g, crude) as a yellow oil. LCMS (ES⁺): m/z 417.2 [M+H]⁺.

Synthesis of tert-butyl 1-(piperidin-4-ylmethyl)piperidine-4-carboxylate. To a mixture of 10% Pd/C (5.00 g, 50.0% purity) in EtOAc (100 mL) was added benzyl 4-((4-(tert-butoxycarbonyl)piperidin-1-yl)methyl)piperidine-1-carboxylate (14.0 g, 33.6 mmol, 1.00 eq) at 20 °C. The mixture was stirred at 20 °C for 12 h under H₂ (15 Psi). LC-MS showed benzyl 4-((4-(tert-butoxycarbonyl)piperidin-1-yl)methyl)piperidine-1-carboxylate was consumed completely and desired mass was detected. The mixture was filtered and filtrate was concentrated under reduced pressure to remove solvent to give the title compound (9.00 g, crude) as a colorless oil. LCMS (ES⁺): m/z 283.2 [M+H]⁺.

Synthesis of tert-butyl 1-((1-(2-fluoro-4-nitrophenyl)piperidin-4-yl)methyl)piperidine-4-carboxylate. To a solution of tert-butyl 1-(piperidin-4-ylmethyl)piperidine-4-carboxylate (9.00 g, 31.9 mmol, 1.00 eq) and 1,2-difluoro-4-nitrobenzene (10.1 g, 63.7 mmol, 7.06 mL, 2.00 eq) in DMF (80.0 mL) was added DIEA (8.24 g, 63.7 mmol, 11.1 mL, 2.00 eq) at 20 °C. The mixture was stirred at 20 °C for 12 h. LC-MS showed that tert-butyl 1-(piperidin-4-ylmethyl)piperidine-4-carboxylate was consumed completely and desired mass was detected. The residue was diluted with H₂O (80 mL) and extracted with EtOAc (60 mL*3). The combined organic layers were washed with brine (80 mL), dried over Na₂SO₄, filtered and concentrated under reduced pressure to give a residue. The residue was purified by column chromatography (SiO₂, Petroleum ether/Ethyl acetate=1/0 to 1/1) to give (9.00 g, crude) as a yellow solid. LCMS (ES⁺): m/z 422.2 [M+H]⁺.

Synthesis of tert-butyl 1-((1-(4-amino-2-fluorophenyl)piperidin-4-yl)methyl)piperidine-4-carboxylate. To a mixture of 10% Pd/C (3.00 g, 50% purity) in EtOAc (100 mL) was added tert-butyl 1-((1-(2-fluoro-4-nitrophenyl)piperidin-4-yl)methyl)piperidine-4-carboxylate (9.00 g, 21.4 mmol, 1.00 eq) at 20 °C. The mixture was stirred at 20 °C for 12 h under H₂ (15 Psi). LC-MS showed tert-butyl 1-((1-(2-fluoro-4-nitrophenyl)piperidin-4-yl)methyl)piperidine-4-carboxylate was consumed completely and desired mass was detected. The mixture was filtered and filtrate was concentrated under reduced pressure to remove solvent to give the title compound (6.2 g, crude) as a pink oil. LCMS (ES⁺): m/z 392.2 [M+H]⁺.

Synthesis of tert-butyl-1-((1-(4-((2,6-dioxopiperidin-3-yl)amino)-2-fluorophenyl)piperidin-4-yl)methyl)piperidine-4-carboxylate. To a solution of tert-butyl 1-((1-(4-amino-2-fluorophenyl)piperidin-4-yl)methyl)piperidine-4-carboxylate (6.00 g, 15.3 mmol, 1.00 eq) and 3-bromopiperidine-2,6-dione (11.8 g, 61.3 mmol, 4.00 eq) in DMF (50.0 mL) was added DIEA (5.94 g, 46.0 mmol, 8.01 mL, 3.00 eq) at 20 °C. The mixture was stirred at 80 °C for 12 h. LC-MS showed that tert-butyl 1-((1-(4-amino-2-fluorophenyl)piperidin-4-yl)methyl)piperidine-4-carboxylate was consumed completely and desired mass was detected. The residue was diluted with H₂O (50 mL) and extracted with EtOAc (40 mL*5). The combined organic layers were washed with brine (50 mL), dried over Na₂SO₄, filtered and concentrated under reduced pressure to give a residue. The residue was purified by column chromatography (SiO₂, Petroleum ether/Ethyl acetate=1/0 to 0/1) to give the title compound (1.00 g, 1.99 mmol, 13.0% yield) as a green solid. LCMS (ES⁺): m/z 503.3 [M+H]⁺.

Synthesis of 1-((1-(4-((2,6-dioxopiperidin-3-yl)amino)-2-fluorophenyl)piperidin-4-yl)methyl)piperidine-4-carboxylic acid. To a solution of tert-butyl-1-((1-(4-((2,6-dioxopiperidin-3-yl)amino)-2-fluorophenyl)piperidin-4-yl)methyl)piperidine-4-carboxylate (700 mg, 1.39 mmol, 1.00 eq) in DCM (5.00 mL) was added TFA (2.30 g, 20.2 mmol, 1.50 mL, 14.5 eq) at 20 °C. The mixture was stirred at 20 °C for 12 h. LC-MS showed that tert-butyl-1-((1-(4-((2,6-dioxopiperidin-3-yl)amino)-2-fluorophenyl)piperidin-4-yl)methyl)piperidine-4-carboxylate was consumed completely and desired mass was detected. The reaction mixture was concentrated under reduced pressure to remove solvent to give the title compound (700 mg, crude) as a green oil. LCMS (ES⁺): m/z 447.2 [M+H]⁺.

Synthesis of rac-1-((1-(4-((2,6-dioxopiperidin-3-yl)amino)-2-fluorophenyl)piperidin-4-yl)methyl)-N-((1r,4r)-4-((5-fluoro-4-(3-(2-oxopyridin-1(2H)-yl)phenyl)pyrimidin-2-yl)amino)cyclohexyl)piperidine-4-carboxamide (Compound 433). To a solution of 1-((1-(4-((2,6-dioxopiperidin-3-yl)amino)-2-fluorophenyl)piperidin-4-yl)methyl)piperidine-4-carboxylic acid (700 mg, 1.57 mmol, 1.00 eq) and trans-1-(3-(2-(((1r,4r)-4-aminocyclohexyl)amino)-5-fluoropyrimidin-4-yl)phenyl)pyridin-2(1H)-one (595 mg, 1.57 mmol, 1.00 eq) in DMF (20 mL) were added DIEA (608 mg, 4.70 mmol, 819 μL, 3.00 eq) and HATU (1.19 g, 3.14 mmol, 2.00 eq) at 20 °C. The mixture was stirred at 20 °C for 12 h. LC-MS showed 1-((1-(4-((2,6-dioxopiperidin-3-yl)amino)-2-fluorophenyl)piperidin-4-yl)methyl)piperidine-4-carboxylic acid was consumed completely desired mass was detected. The residue was diluted with H₂O (20 mL) and extracted with EtOAc (20 mL*3). The combined organic layers were washed with brine (20 mL), dried over NA₂SO₄, filtered and concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 30%-60% B over 8.0 min) to give the title compound (Compound 433) (98.0 mg, 120 μmol, 7.68% yield, 99.2% purity) as a white solid. LCMS (ES⁺): m/z 808.3 [M+H]⁺.

### Example 366. Synthesis of trans-4-(4-(3-(2,6-dioxopiperidin-3-yl)-1-methyl-1H-indazol-6-yl)piperazin-1-yl)-N-(trans-4-((5-fluoro-4-(3-(2-oxopyridin-1(2H)-yl)phenyl)pyrimidin-2-yl)amino)cyclohexyl)cyclohexane-1-carboxamide (Compound 388)

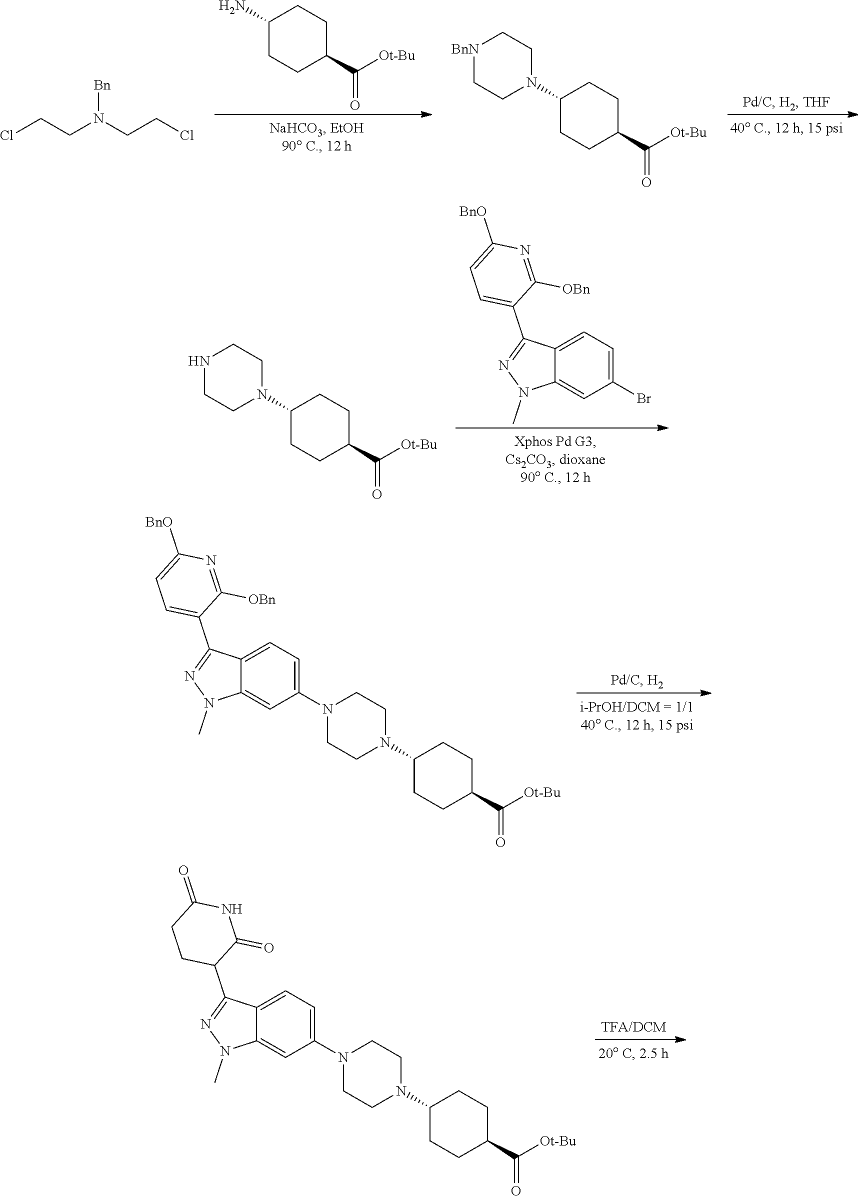

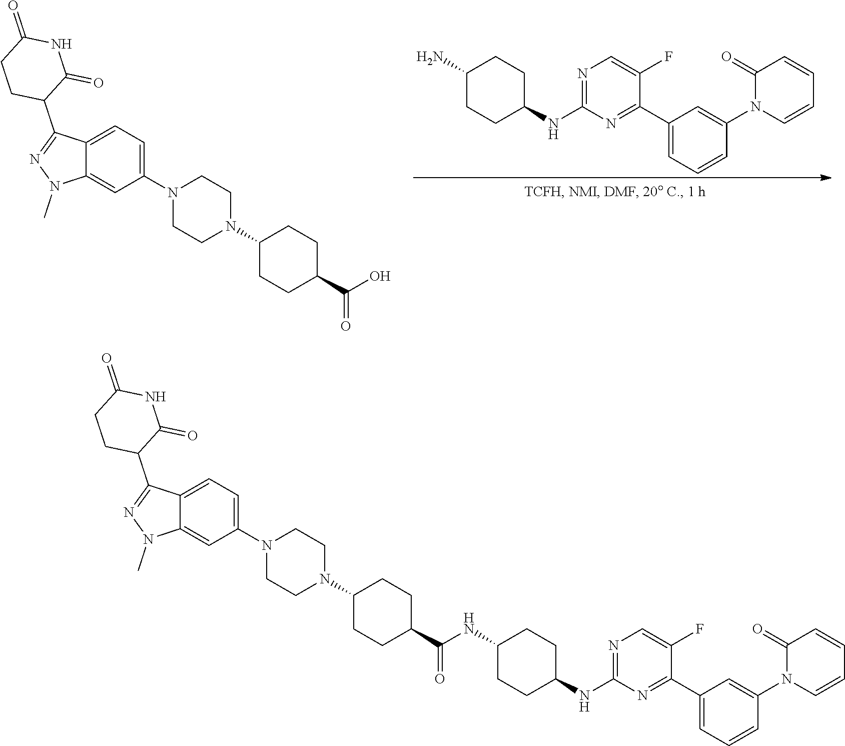 [SMILES: Cn1nc(C2CCC(=O)NC2=O)c2ccc(N3CCN([C@H]4CC[C@H](C(=O)O)CC4)CC3)cc21 ; N[C@H]1CC[C@H](Nc2ncc(F)c(-c3cccc(-n4ccccc4=O)c3)n2)CC1 ; Cn1nc(C2CCC(=O)NC2=O)c2ccc(N3CCN([C@H]4CC[C@H](C(=O)N[C@H]5CC[C@H](Nc6ncc(F)c(-c7cccc(-n8ccccc8=O)c7)n6)CC5)CC4)CC3)cc21]

Synthesis of trans-tert-butyl 4-(4-benzylpiperazin-1-yl)cyclohexane-1-carboxylate. To a solution of N-benzyl-2-chloro-N-(2-chloroethyl)ethan-1-amine (5.39 g, 20.1 mmol, 1.00 eq, HCl) and trans-tert-butyl 4-aminocyclohexane-1-carboxylate (4.00 g, 20.1 mmol, 1.00 eq) in EtOH (80 mL) was added NaHCO₃ (4.22 g, 50.2 mmol, 1.95 mL, 2.50 eq) at 20 °C, the mixture was stirred at 90 °C for 12 h. LCMS showed N-benzyl-2-chloro-N-(2-chloroethyl)ethan-1-amine was consumed and the desired target MS was detected. The mixture was concentrated under reduced pressure to give a residue. H₂O (80 mL) was added to the residue and the mixture was extracted with EtOAc (80 mL*3), then the combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (4.00 g, 11.2 mmol, 55.6% yield) as a white solid. LCMS (ES⁺): m/z 359.2 [M+H]⁺

Synthesis of trans-tert-butyl 4-(piperazin-1-yl)cyclohexane-1-carboxylate. To a solution of trans-tert-butyl 4-(4-benzylpiperazin-1-yl)cyclohexane-1-carboxylate (4.00 g, 11.2 mmol, 1.00 eq) in THE (40 mL) was added 10% Pd/C (12.0 g, 50% purity) at 20 °C under N₂. The mixture was degassed and purged with H₂ for three times and the mixture was stirred at 40 °C for 12 h under H₂ (15 psi). LCMS showed trans-tert-butyl 4-(4-benzylpiperazin-1-yl)cyclohexane-1-carboxylate was consumed and the desired target MS was detected. The mixture was filtered, the filtrate was concentrated under reduced pressure to give the title compound (3.00 g, crude) as a yellow oil. LCMS (ES⁺): m/z 269.2 [M+H]⁺

Synthesis of trans-tert-butyl 4-(4-(3-(2,6-bis(benzyloxy)pyridin-3-yl)-1-methyl-1H-indazol-6-yl)piperazin-1-yl)cyclohexane-1-carboxylate. To a solution of trans-tert-butyl 4-(piperazin-1-yl)cyclohexane-1-carboxylate (2.90 g, 10.8 mmol, 1.00 eq) and 3-(2,6-bis(benzyloxy)pyridin-3-yl)-6-bromo-1-methyl-1H-indazole (5.41 g, 10.8 mmol, 1.00 eq) in dioxane (150 mL) were added Cs₂CO₃ (7.04 g, 21.6 mmol, 2.00 eq) and XPhos Pd G3 (915 mg, 1.08 mmol, 0.100 eq) at 20 °C under N₂, the mixture was stirred at 90 °C for 12 h under N₂. LCMS showed trans-tert-butyl 4-(piperazin-1-yl)cyclohexane-1-carboxylate was consumed and the desired target MS was detected. H₂O (150 mL) was added to the mixture, the mixture was extracted with EtOAc (100 mL*3), then the combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (2.90 g, 4.22 mmol, 39.0% yield) as a white solid. LCMS (ES⁺): m/z 688.3 [M+H]⁺

Synthesis of trans-tert-butyl 4-(4-(3-(2,6-dioxopiperidin-3-yl)-1-methyl-1H-indazol-6-yl)piperazin-1-yl)cyclohexane-1-carboxylate. To a solution of trans-tert-butyl 4-(4-(3-(2,6-bis(benzyloxy)pyridin-3-yl)-1-methyl-1H-indazol-6-yl)piperazin-1-yl)cyclohexane-1-carboxylate (2.40 g, 3.49 mmol, 1.00 eq) in i-PrOH (100 mL) and DCM (100 mL) was added 10% Pd/C (2.40 g, 50.0% purity) at 20 °C under N₂. The mixture was degassed and purged with H₂ for three times and the mixture was stirred at 40 °C for 12 h under H₂ (15 psi). LCMS showed trans-tert-butyl 4-(4-(3-(2,6-bis(benzyloxy)pyridin-3-yl)-1-methyl-1H-indazol-6-yl)piperazin-1-yl)cyclohexane-1-carboxylate was consumed and the desired target MS was detected. The mixture was filtered, the filtrate was concentrated under reduced pressure to give the title compound (2.00 g, crude) as a white solid. LCMS (ES⁺): m/z 510.4 [M+H]⁺

Synthesis of trans-4-(4-(3-(2,6-dioxopiperidin-3-yl)-1-methyl-1H-indazol-6-yl)piperazin-1-yl)cyclohexane-1-carboxylic acid A solution of trans-tert-butyl 4-(4-(3-(2,6-dioxopiperidin-3-yl)-1-methyl-1H-indazol-6-yl)piperazin-1-yl)cyclohexane-1-carboxylate (0.800 g, 1.57 mmol, 1.00 eq) in TFA (2 mL) and DCM (6 mL) was stirred at 20 °C for 2.5 h. LCMS showed trans-tert-butyl 4-(4-(3-(2,6-dioxopiperidin-3-yl)-1-methyl-1H-indazol-6-yl)piperazin-1-yl)cyclohexane-1-carboxylate was consumed and the desired target MS was detected. The mixture was concentrated under reduced pressure to give the title compound (820 mg, crude) as a yellow oil. LCMS (ES⁺): m/z 454.3 [M+H]⁺

Synthesis of trans-4-(4-(3-(2,6-dioxopiperidin-3-yl)-1-methyl-1H-indazol-6-yl)piperazin-1-yl)-N-(trans-4-((5-fluoro-4-(3-(2-oxopyridin-1(2H)-yl)phenyl)pyrimidin-2-yl)amino)cyclohexyl)cyclohexane-1-carboxamide (Compound 388) To a solution of trans-4-(4-(3-(2,6-dioxopiperidin-3-yl)-1-methyl-1H-indazol-6-yl)piperazin-1-yl)cyclohexane-1-carboxylic acid (0.800 g, 1.76 mmol, 1.00 eq) and trans-1-(3-(2-((4-aminocyclohexyl)amino)-5-fluoropyrimidin-4-yl)phenyl)pyridin-2(1H)-one (609 mg, 1.23 mmol, 0.700 eq, TFA) in DMF (10 mL) were added TCFH (544 mg, 1.94 mmol, 1.10 eq) and NMI (449 mg, 5.47 mmol, 436 μL, 3.10 eq) at 20 °C, the mixture was stirred at 20 °C for 1 h. LCMS showed trans-4-(4-(3-(2,6-dioxopiperidin-3-yl)-1-methyl-1H-indazol-6-yl)piperazin-1-yl)cyclohexane-1-carboxylic acid was consumed and the desired target MS was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 20%-65% B over 8.0 min) to give the title compound (Compound 388) (17.7 mg, 19.8 μmol, 1.12% yield, 91.0% purity) as a green solid. LCMS (ES⁺): m/z 815.3 [M+H]⁺

### Example 367. Synthesis of 3-(5-(1-((1-(2-(4-((5-fluoro-4-(3-(2-oxopyridin-1(2H)-yl)phenyl)pyrimidin-2-yl)amino)piperidin-1-yl)-2-oxoethyl)piperidin-4-yl)methyl)piperidin-4-yl)-3-methyl-2-oxo-2,3-dihydro-1H-benzo[d]imidazol-1-yl)piperidine-2,6-dione (Compound 312)

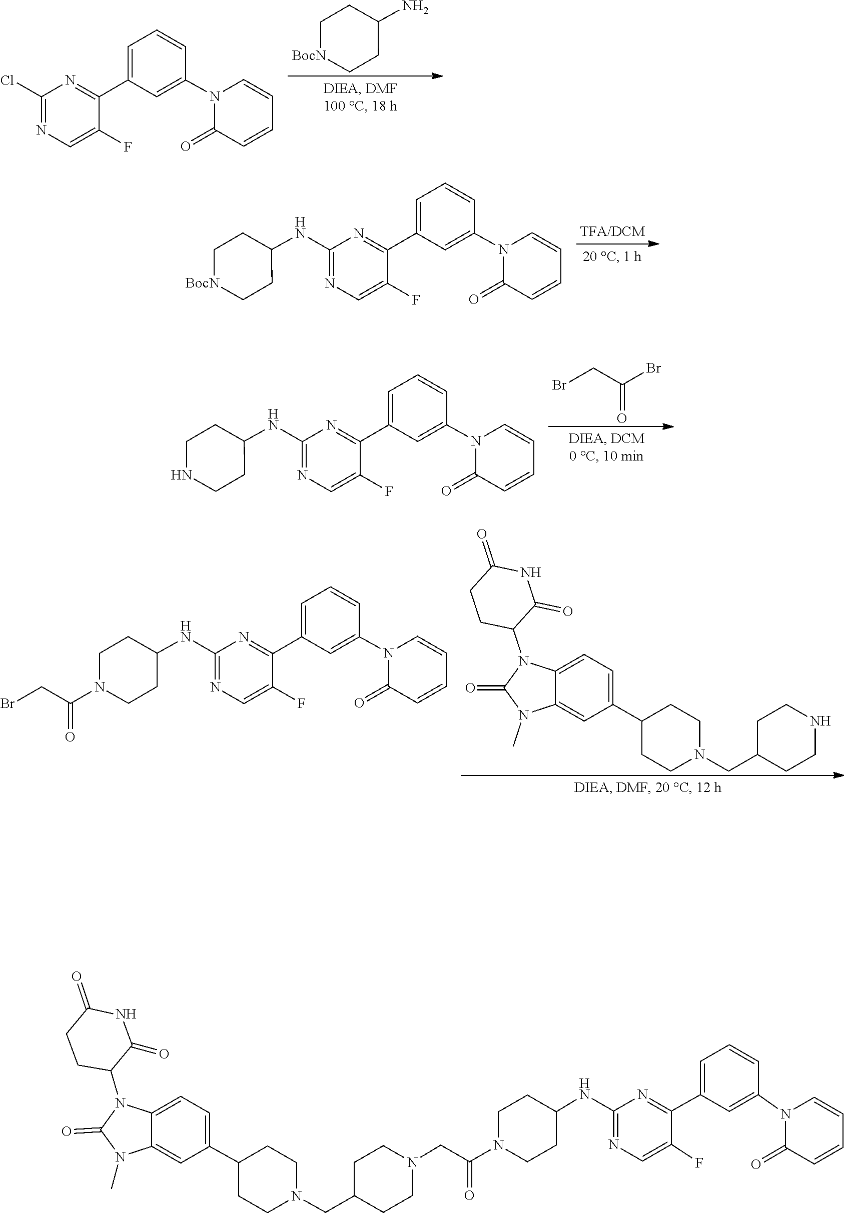

Synthesis of tert-butyl 4-((5-fluoro-4-(3-(2-oxopyridin-1(2H)-yl)phenyl)pyrimidin-2-yl)amino)piperidine-1-carboxylate. To a solution of tert-butyl 4-aminopiperidine-1-carboxylate (996 mg, 4.97 mmol, 1.50 eq) in DMF (10 mL) were added DIEA (1.29 g, 9.94 mmol, 1.73 mL, 3.00 eq) and 1-(3-(2-chloro-5-fluoropyrimidin-4-yl)phenyl)pyridin-2(1H)-one (1.00 g, 3.31 mmol, 1.00 eq) at 20 °C. The mixture was stirred at 100 °C for 12 h. LC-MS showed 1-(3-(2-chloro-5-fluoropyrimidin-4-yl)phenyl)pyridin-2(1H)-one remained and the desired mass was detected. Then tert-butyl 4-aminopiperidine-1-carboxylate (996 mg, 4.97 mmol, 1.50 eq) was added to the mixture at 20 °C. The mixture was stirred at 100 °C for 6 h. LC-MS showed 1-(3-(2-chloro-5-fluoropyrimidin-4-yl)phenyl)pyridin-2(1H)-one was consumed and the desired mass was detected. Water (10 mL) was added to the mixture, then the mixture was extracted with EtOAc (10 mL*3). The combined organic layers were dried over anhydrous sodium sulfate, filtered and the filtrate was concentrated under reduced pressure to give a residue. The residue was purified by MPLC (SiO₂, PE/EtOAc=1/1 to 0/1) to give the title compound (980 mg, 2.11 mmol, 63.5% yield) as a white solid. LCMS (ES+): m/z 466.3 [M+H]⁺

Synthesis of 1-(3-(5-fluoro-2-(piperidin-4-ylamino)pyrimidin-4-yl)phenyl)pyridin-2(1H)-one. A solution of tert-butyl 4-((5-fluoro-4-(3-(2-oxopyridin-1(2H)-yl)phenyl)pyrimidin-2-yl)amino)piperidine-1-carboxylate (830 mg, 1.78 mmol, 1.00 eq) in DCM (6 mL) and TFA (2 mL) was stirred at 20 °C for 1 h. LC-MS showed tert-butyl 4-((5-fluoro-4-(3-(2-oxopyridin-1(2H)-yl)phenyl)pyrimidin-2-yl)amino)piperidine-1-carboxylate was consumed completely and the desired mass was detected. The reaction mixture was concentrated under reduced pressure to give the title compound (854 mg, crude, TFA) as a yellow oil. LCMS (ES⁺): m/z 366.1 [M+H]⁺

Synthesis of 1-(3-(2-((1-(2-bromoacetyl)piperidin-4-yl)amino)-5-fluoropyrimidin-4-yl)phenyl)pyridin-2(1H)-one. To a solution of 1-(3-(5-fluoro-2-(piperidin-4-ylamino)pyrimidin-4-yl)phenyl)pyridin-2(1H)-one (850 mg, 2.33 mmol, 1.00 eq) and DIEA (901.93 mg, 6.98 mmol, 1.22 mL, 3.00 eq) in DCM (10 mL) was added 2-bromoacetyl bromide (704 mg, 3.49 mmol, 304 μL, 1.50 eq) at 0 °C. The mixture was stirred at 0 °C for 10 min. LC-MS showed 1-(3-(5-fluoro-2-(piperidin-4-ylamino)pyrimidin-4-yl)phenyl)pyridin-2(1H)-one was consumed completely and the desired mass was detected. Water (10 mL) was added to the mixture at 20 °C. Then the reaction mixture was extracted with EtOAc (5 mL*3). The organic layers were dried over anhydrous sodium sulfate, filtered and the filtrate was concentrated under reduced pressure to give a residue. The residue was purified by MPLC (SiO₂, PE/EtOAc=3/1 to 1/1) to give the title compound (400 mg, 822 μmol, 35.4% yield) as a yellow oil. LCMS (ES⁺): m/z 486.1 [M+H]⁺

Synthesis of 3-(5-(1-((1-(2-(4-((5-fluoro-4-(3-(2-oxopyridin-1(2H)-yl)phenyl)pyrimidin-2-yl)amino)piperidin-1-yl)-2-oxoethyl)piperidin-4-yl)methyl)piperidin-4-yl)-3-methyl-2-oxo-2,3-dihydro-1H-benzo[d]imidazol-1-yl)piperidine-2,6-dione (Compound 312). To a stirred solution of 3-(3-methyl-2-oxo-5-(1-(piperidin-4-ylmethyl)piperidin-4-yl)-2,3-dihydro-1H-benzo[d]imidazol-1-yl)piperidine-2,6-dione (110 mg, 199 μmol, 1.00 eq, TFA) in DMF (4 mL) were added DIEA (25.7 mg, 199 μmol, 34.6 μL, 1.00 eq) and 1-(3-(2-((1-(2-bromoacetyl)piperidin-4-yl)amino)-5-fluoropyrimidin-4-yl)phenyl)pyridin-2(1H)-one (96.6 mg, 199 μmol, 1.00 eq) at 20 °C. The mixture was stirred at 20 °C for 12 h. LCMS showed -(3-methyl-2-oxo-5-(1-(piperidin-4-ylmethyl)piperidin-4-yl)-2,3-dihydro-1H-benzo[d]imidazol-1-yl)piperidine-2,6-dione was consumed completely and the desired mass was detected. The mixture was diluted with H₂O (5 mL) and then extracted with EtOAc (4 mL*3). The combined organic layers were dried over Na₂SO₄, filtered and the filtrate was concentrated under reduced pressure to give a residue. The residue was purified by prep-HPLC (column: Phenomenex luna C18 100*40 mm*3 um; mobile phase: [H₂O (0.2% FA)-ACN]; gradient: 10%-35% B over 8.0 min) to give the title compound (Compound 312) (5.30 mg, 6.23 μmol, 3.14% yield, 99.4% purity) as a white solid. LCMS (ES⁺): m/z 845.3 [M+H]⁺

### Example 368. Synthesis of 3-((3-fluoro-4-(1-((1-(2-(4-((5-fluoro-4-(3-(2-oxopyridin-1(2H)-yl)phenyl)pyrimidin-2-yl)amino)piperidin-1-yl)-2-oxoethyl)piperidin-4-yl)methyl)piperidin-4-yl)phenyl)amino)piperidine-2,6-dione (Compound 314)

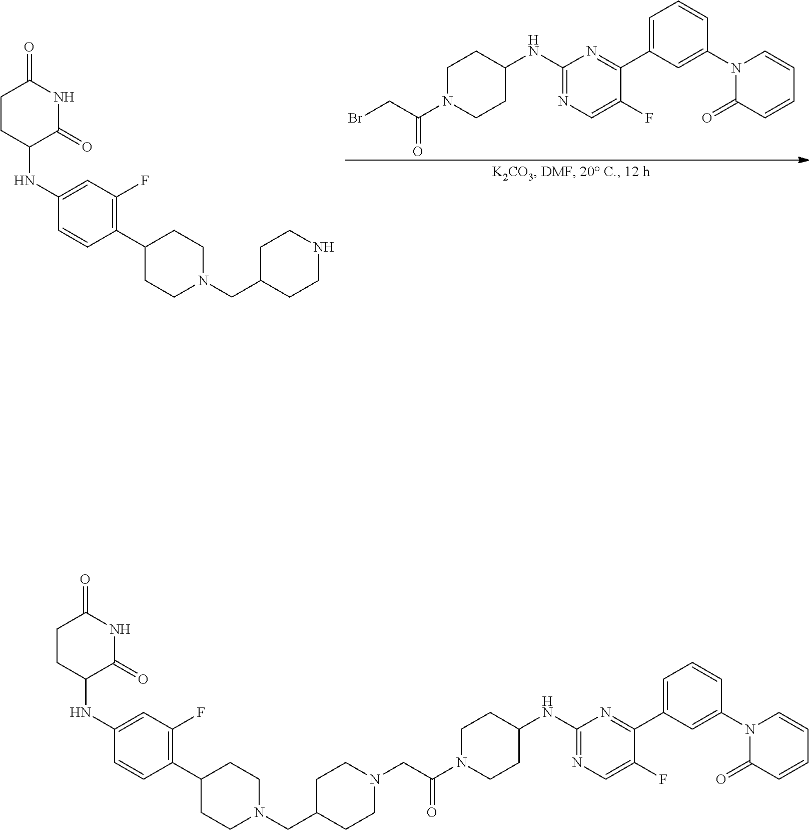 [SMILES: O=C1CCC(Nc2ccc(C3CCN(CC4CCNCC4)CC3)c(F)c2)C(=O)N1 ; O=C(CBr)N1CCC(Nc2ncc(F)c(-c3cccc(-n4ccccc4=O)c3)n2)CC1 ; O=C1CCC(Nc2ccc(C3CCN(CC4CCN(CC(=O)N5CCC(Nc6ncc(F)c(-c7cccc(-n8ccccc8=O)c7)n6)CC5)CC4)CC3)c(F)c2)C(=O)N1]

Synthesis of 3-((3-fluoro-4-(1-((1-(2-(4-((5-fluoro-4-(3-(2-oxopyridin-1(2H)-yl)phenyl)pyrimidin-2-yl)amino)piperidin-1-yl)-2-oxoethyl)piperidin-4-yl)methyl)piperidin-4-yl)phenyl)amino)piperidine-2,6-dione (Compound 314). To a solution of 3-((3-fluoro-4-(1-(piperidin-4-ylmethyl)piperidin-4-yl)phenyl)amino)piperidine-2,6-dione (133 mg, 257 μmol, 1.25 eq, TFA) and 1-(3-(2-((1-(2-bromoacetyl)piperidin-4-yl)amino)-5-fluoropyrimidin-4-yl)phenyl)pyridin-2(1H)-one (100 mg, 206 μmol, 1.00 eq) in DMF (4 mL) was added K₂CO₃ (142 mg, 1.03 mmol, 5.00 eq) at 20 °C, then the mixture was stirred at 20 °C for 12 h. LCMS showed 3-((3-fluoro-4-(1-(piperidin-4-ylmethyl)piperidin-4-yl)phenyl)amino)piperidine-2,6-dione was consumed and the desired target MS was detected. The mixture was filtered and the filtrate was concentrated under reduced pressure to give a residue. The residue was purified by prep-HPLC (column: Phenomenex luna C18 100*40 mm*3 um; mobile phase: [H₂O (0.2% FA)-ACN]; gradient: 1%-35% B over 8.0 min) to give the title compound (Compound 314) (16.1 mg, 19.9 μmol, 9.69% yield, 100% purity) as a gray solid. LCMS (ES⁺): m/z 808.4 [M+H]⁺

### Example 369. Synthesis of 3-(6-(1′-(3-(4-((5-fluoro-4-(3-(2-oxopyridin-1(2H)-yl)phenyl)pyrimidin-2-yl)amino)piperidin-1-yl)-3-oxopropyl)-[1,4′-bipiperidin]-4-yl)-2-oxobenzo[d]oxazol-3 (2H)-yl)piperidine-2,6-dione (Compound 353)

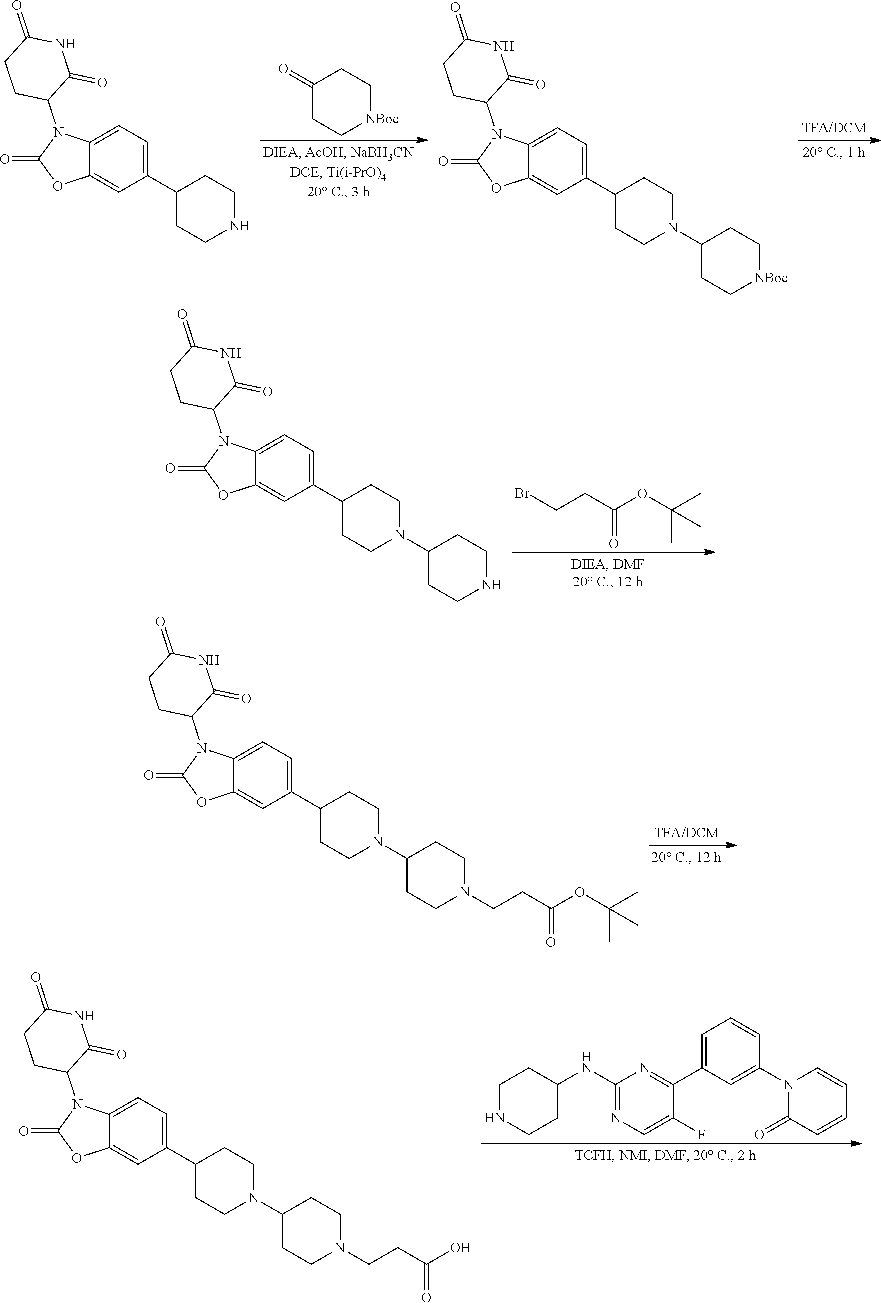

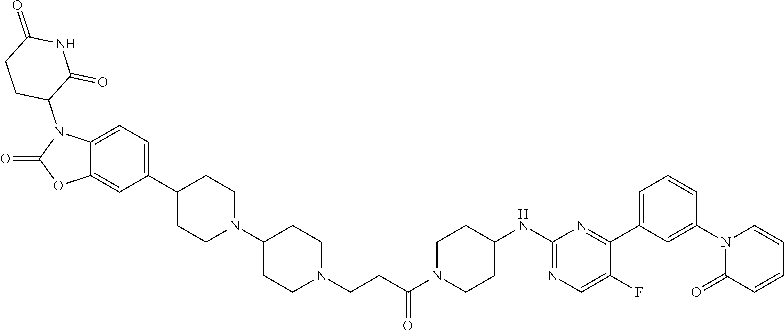 [SMILES: O=C1CCC(n2c(=O)oc3cc(C4CCN(C5CCN(CCC(=O)N6CCC(Nc7ncc(F)c(-c8cccc(-n9ccccc9=O)c8)n7)CC6)CC5)CC4)ccc32)C(=O)N1]

Synthesis of tert-butyl 4-(3-(2,6-dioxopiperidin-3-yl)-2-oxo-2,3-dihydrobenzo[d]oxazol-6-yl)-[1,4′-bipiperidine]-1′-carboxylate. To a solution of 3-(2-oxo-6-(piperidin-4-yl)benzo[d]oxazol-3 (2H)-yl)piperidine-2,6-dione (5.00 g, 11.3 mmol, 1.00 eq, TFA) in DCE (100 mL) was added DIEA to adjust pH to 8 at 20 °C. The mixture was stirred at 20 °C for 0.5 h. AcOH (2.03 g, 33.8 mmol, 1.94 mL, 3.00 eq), Ti(i-PrO)₄ (16.0 g, 56.4 mmol, 16.6 mL, 5.00 eq) and tert-butyl 4-oxopiperidine-1-carboxylate (6.74 g, 33.8 mmol, 3.00 eq) were added to the mixture at 20 °C, pH was 5 now. The mixture was stirred at 20 °C for 0.5 h. Then NaBH₃CN (3.54 g, 56.4 mmol, 5.00 eq) was added to the mixture at 20 °C. The mixture was stirred at 20 °C for 2 h. LCMS showed the desired mass was detected. Water (50 mL) was added to the mixture at 20 °C. Solis was precipitated and the mixture was filtered. The filter cake was dried under reduced pressure to give the title compound (5.50 g, crude) as a yellow solid. LCMS (ES⁺): m/z 513.3 [M+H]⁺.

Synthesis of 3-(6-([1,4′-bipiperidin]-4-yl)-2-oxobenzo[d]oxazol-3 (2H)-yl)piperidine-2,6-dione. To a solution of tert-butyl 4-(3-(2,6-dioxopiperidin-3-yl)-2-oxo-2,3-dihydrobenzo[d]oxazol-6-yl)-[1,4′-bipiperidine]-1′-carboxylate (3.00 g, 5.85 mmol, 1.00 eq) in DCM (30 mL) was added TFA (6 mL) at 20 °C. The mixture was stirred at 20 °C for 1 h. LCMS showed tert-butyl 4-(3-(2,6-dioxopiperidin-3-yl)-2-oxo-2,3-dihydrobenzo[d]oxazol-6-yl)-[1,4′-bipiperidine]-1′-carboxylate was consumed completely and the desired mass was detected. The reaction mixture was concentrated under reduced pressure to give the title compound (3.00 g, crude, TFA) as a yellow oil. LCMS (ES⁺): m/z 413.3 [M+H]⁺.

Synthesis of tert-butyl 3-(4-(3-(2,6-dioxopiperidin-3-yl)-2-oxo-2,3-dihydrobenzo[d]oxazol-6-yl)-[1,4′-bipiperidin]-1′-yl)propanoate. To a solution of 3-(6-([1,4′-bipiperidin]-4-yl)-2-oxobenzo[d]oxazol-3 (2H)-yl)piperidine-2,6-dione (300 mg, 570 μmol, 1.00 eq, TFA) in DMF (8 mL) were added DIEA (368 mg, 2.85 mmol, 496 μL, 5.00 eq) and tert-butyl 3-bromopropanoate (477 mg, 2.28 mmol, 380 μL, 4.00 eq) at 20 °C. The mixture was stirred at 20 °C for 12 h. LCMS showed 3-(6-([1,4′-bipiperidin]-4-yl)-2-oxobenzo[d]oxazol-3 (2H)-yl)piperidine-2,6-dione was consumed completely and the desired mass was detected. The reaction mixture was filtered and the filtrate was concentrated under reduced pressure to give a residue. The residue was purified by prep-TLC (SiO₂, Ethyl acetate:Methanol=10:1) to give the crude product. The crude product was purified by prep-HPLC (column: Waters Xbridge BEH C18 250*70 mm*10 um; mobile phase: [H₂O (10 mM NH₄HCO₃)-ACN]; gradient: 35%-65% B over 20.0 min) to give the title compound (70.0 mg, 129 μmol, 2.27% yield) as a white solid. LCMS (ES⁺): m/z 541.4 [M+H]⁺.

Synthesis of 3-(4-(3-(2,6-dioxopiperidin-3-yl)-2-oxo-2,3-dihydrobenzo[d]oxazol-6-yl)-[1,4′-bipiperidin]-1′-yl)propanoic acid. To a solution of tert-butyl 3-(4-(3-(2,6-dioxopiperidin-3-yl)-2-oxo-2,3-dihydrobenzo[d]oxazol-6-yl)-[1,4′-bipiperidin]-1′-yl)propanoate (60.0 mg, 111 μmol, 1.00 eq) in DCM (1.5 mL) was added TFA (0.5 mL) at 20 °C. The mixture was stirred at 20 °C for 12 h. LCMS showed tert-butyl 3-(4-(3-(2,6-dioxopiperidin-3-yl)-2-oxo-2,3-dihydrobenzo[d]oxazol-6-yl)-[1,4′-bipiperidin]-1′-yl)propanoate was consumed completely and the desired mass was detected. The reaction mixture was concentrated under reduced pressure to give the title compound (50.0 mg, crude) as a colorless oil. LCMS (ES⁺): m/z 485.2 [M+H]⁺.

Synthesis of 3-(6-(1′-(3-(4-((5-fluoro-4-(3-(2-oxopyridin-1(2H)-yl)phenyl)pyrimidin-2-yl)amino)piperidin-1-yl)-3-oxopropyl)-[1,4′-bipiperidin]-4-yl)-2-oxobenzo[d]oxazol-3 (2H)-yl)piperidine-2,6-dione (Compound 353). To a solution of 3-(4-(3-(2,6-dioxopiperidin-3-yl)-2-oxo-2,3-dihydrobenzo[d]oxazol-6-yl)-[1,4′-bipiperidin]-1′-yl)propanoic acid (50.0 mg, 103 μmol, 1.00 eq) in DMF (1 mL) were added TCFH (63.7 mg, 227 μmol, 2.20 eq), NMI (50.8 mg, 619 μmol, 49.4 μL, 6.00 eq) and 1-[3-[5-fluoro-2-(4-piperidylamino)pyrimidin-4-yl]phenyl]pyridin-2-one (41.5 mg, 103 μmol, 1.00 eq, HCl) at 20 °C. The mixture was stirred at 20 °C for 2 h. LCMS showed 3-(4-(3-(2,6-dioxopiperidin-3-yl)-2-oxo-2,3-dihydrobenzo[d]oxazol-6-yl)-[1,4′-bipiperidin]-1′-yl)propanoic acid was consumed completely and the desired mass was detected. The reaction mixture was filtered and the filtrate was concentrated under reduced pressure to give a residue. The residue was purified by prep-HPLC (column: Phenomenex Luna C18 75*30 mm*3 um; mobile phase: [H₂O (0.2% FA)-ACN]; gradient: 10%-50% B over 8.0 min) to give the title compound (Compound 353) (4.60 mg, 5.16 μmol, 5.00% yield, 98.6% purity, FA) as a white solid. LCMS (ES⁺): m/z 832.1[M+H]⁺

### Example 370. Synthesis of 3-((3-fluoro-4-(1′-(3-(4-((5-fluoro-4-(3-(2-oxopyridin-1(2H)-yl)phenyl)pyrimidin-2-yl)amino)piperidin-1-yl)-3-oxopropyl)-[1,4′-bipiperidin]-4-yl)phenyl)amino)piperidine-2,6-dione (Compound 343)

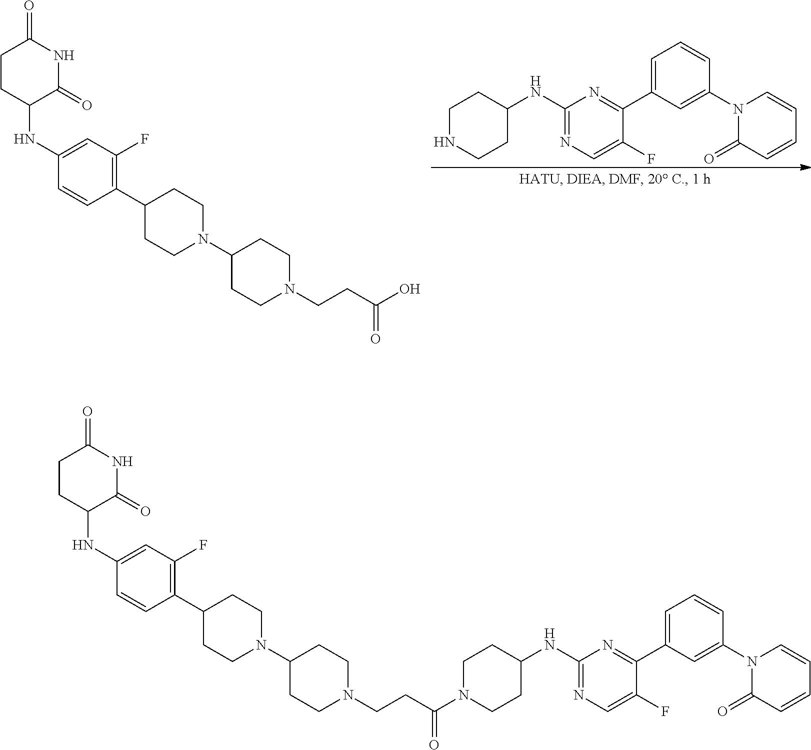 [SMILES: O=C(O)CCN1CCC(N2CCC(c3ccc(NC4CCC(=O)NC4=O)cc3F)CC2)CC1 ; O=c1ccccn1-c1cccc(-c2nc(NC3CCNCC3)ncc2F)c1 ; O=C1CCC(Nc2ccc(C3CCN(C4CCN(CCC(=O)N5CCC(Nc6ncc(F)c(-c7cccc(-n8ccccc8=O)c7)n6)CC5)CC4)CC3)c(F)c2)C(=O)N1]

Synthesis of 3-((3-fluoro-4-(1′-(3-(4-((5-fluoro-4-(3-(2-oxopyridin-1(2H)-yl)phenyl)pyrimidin-2-yl)amino)piperidin-1-yl)-3-oxopropyl)-[1,4′-bipiperidin]-4-yl)phenyl)amino)piperidine-2,6-dione (Compound 343). To a solution of 3-(4-(4-((2,6-dioxopiperidin-3-yl)amino)-2-fluorophenyl)-[1,4′-bipiperidin]-1′-yl)propanoic acid (42.0 mg, 91.2 μmol, 1.00 eq) in DMF (2 mL) were added HATU (41.6 mg, 109 μmol, 1.20 eq) and DIEA (58.9 mg, 456 μmol, 79.4 μL, 5.00 eq) at 20 °C, then 1-(3-(5-fluoro-2-(piperidin-4-ylamino)pyrimidin-4-yl)phenyl)pyridin-2(1H)-one (35.0 mg, 73.0 μmol, 0.800 eq, TFA) was added to the mixture. The mixture was stirred at 20 °C for 1 h. LCMS showed 3-(4-(4-((2,6-dioxopiperidin-3-yl)amino)-2-fluorophenyl)-[1,4′-bipiperidin]-1′-yl)propanoic acid was consumed and the desired mass was detected. To the mixture was added H₂O (3 mL) and the mixture was extracted with EtOAc (3 mL*3). The combined organic layers were washed with brine (3 mL*3), dried over Na₂SO₄, filtered and the filtrate was concentrated under reduced pressure to give a residue. The residue was purified by prep-HPLC (column: Phenomenex Luna C18 75*30 mm*3 um; mobile phase: [H₂O (0.1% TFA)-ACN]; gradient: 5%-35% B over 8.0 min) to give the title compound (Compound 343) (8.8 mg, 9.29 μmol, 10.2% yield, 97.3% purity, TFA) as a yellow solid. LCMS (ES⁺): m/z 808.4 [M+H]⁺

### Example 371. Synthesis of 3-(6-(1-((1-(2-(4-((5-fluoro-4-(3-(2-oxopyridin-1(2H)-yl)phenyl)pyrimidin-2-yl)amino)piperidin-1-yl)-2-oxoethyl)piperidin-4-yl)methyl)piperidin-4-yl)-2-oxobenzo[d]oxazol-3 (2H)-yl)piperidine-2,6-dione (Compound 303)

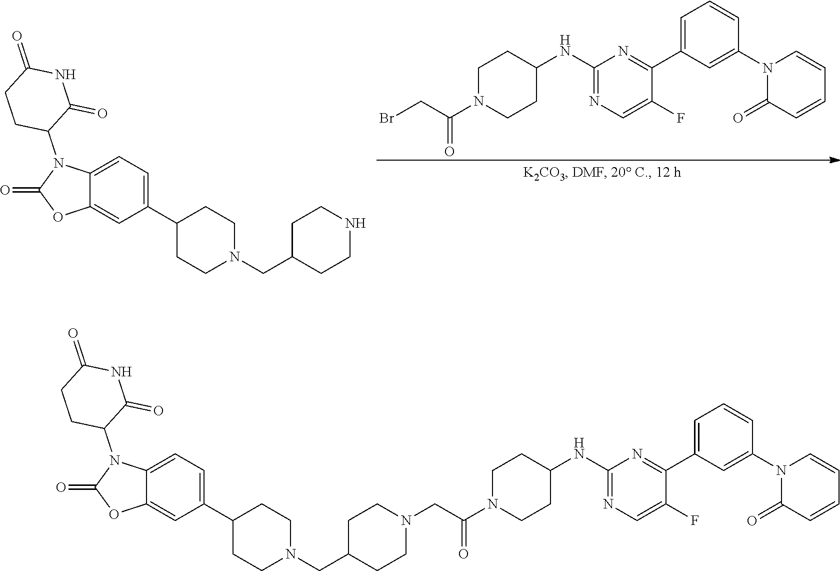 [SMILES: O=C1CCC(n2c(=O)oc3cc(C4CCN(CC5CCNCC5)CC4)ccc32)C(=O)N1 ; O=C(CBr)N1CCC(Nc2ncc(F)c(-c3cccc(-n4ccccc4=O)c3)n2)CC1 ; O=C1CCC(n2c(=O)oc3cc(C4CCN(CC5CCN(CC(=O)N6CCC(Nc7ncc(F)c(-c8cccc(-n9ccccc9=O)c8)n7)CC6)CC5)CC4)ccc32)C(=O)N1]

Synthesis of 3-(6-(1-((1-(2-(4-((5-fluoro-4-(3-(2-oxopyridin-1(2H)-yl)phenyl)pyrimidin-2-yl)amino)piperidin-1-yl)-2-oxoethyl)piperidin-4-yl)methyl)piperidin-4-yl)-2-oxobenzo[d]oxazol-3 (2H)-yl)piperidine-2,6-dione (Compound 303). To a solution of 3-(2-oxo-6-(1-(piperidin-4-ylmethyl)piperidin-4-yl)benzo[d]oxazol-3 (2H)-yl)piperidine-2,6-dione (95.0 mg, 205 μmol, 1.00 eq, HCl) in DMF (4 mL) were added K₂CO₃ (142 mg, 1.03 mmol, 5.00 eq) and 1-[3-[2-[[1-(2-bromoacetyl)-4-piperidyl]amino]-5-fluoro-pyrimidin-4-yl]phenyl]pyridin-2-one (99.8 mg, 205 μmol, 1.00 eq) at 20 °C. The mixture was stirred at 20 °C for 12 h. LCMS showed 3-(2-oxo-6-(1-(piperidin-4-ylmethyl)piperidin-4-yl)benzo[d]oxazol-3 (2H)-yl)piperidine-2,6-dione was consumed completely and the desired mass was detected. The reaction mixture was filtered and the filtrate was concentrated under reduced pressure to give a residue. The residue was purified by prep-HPLC (column: Phenomenex luna C18 100*40 mm*3 um; mobile phase: [H₂O (0.2% FA)-ACN]; gradient: 1%-35% B over 8.0 min) to give the title compound (Compound 303) (5.40 mg, 6.08 μmol, 2.96% yield, 98.8% purity, FA) as a white solid. LCMS (ES⁺): m/z 832.4[M+H]⁺

### Example 372. Synthesis of 3-(6-(1-((1-(2-(4-((5-fluoro-4-(3-(2-oxopyridin-1(2H)-yl)phenyl)pyrimidin-2-yl)amino)piperidin-1-yl)-2-oxoethyl)piperidin-4-yl)methyl)piperidin-4-yl)-1-methyl-1H-indazol-3-yl)piperidine-2,6-dione (Compound 315)

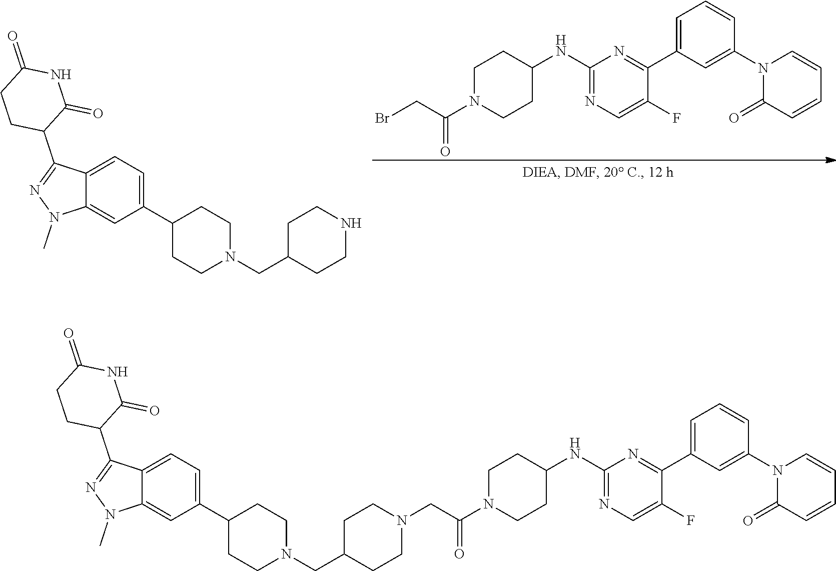 [SMILES: Cn1nc(C2CCC(=O)NC2=O)c2ccc(C3CCN(CC4CCNCC4)CC3)cc21 ; O=C(CBr)N1CCC(Nc2ncc(F)c(-c3cccc(-n4ccccc4=O)c3)n2)CC1 ; Cn1nc(C2CCC(=O)NC2=O)c2ccc(C3CCN(CC4CCN(CC(=O)N5CCC(Nc6ncc(F)c(-c7cccc(-n8ccccc8=O)c7)n6)CC5)CC4)CC3)cc21]

Synthesis of 3-(6-(1-((1-(2-(4-((5-fluoro-4-(3-(2-oxopyridin-1(2H)-yl)phenyl)pyrimidin-2-yl)amino)piperidin-1-yl)-2-oxoethyl)piperidin-4-yl)methyl)piperidin-4-yl)-1-methyl-1H-indazol-3-yl)piperidine-2,6-dione (Compound 315). To a solution of 3-(1-methyl-6-(1-(piperidin-4-ylmethyl)piperidin-4-yl)-1H-indazol-3-yl)piperidine-2,6-dione (111 mg, 206 μmol, 1.00 eq, TFA) in DMF (3 mL) were added DIEA (26.6 mg, 206 μmol, 35.8 μL, 1.00 eq) and 1-(3-(2-((1-(2-bromoacetyl)piperidin-4-yl)amino)-5-fluoropyrimidin-4-yl)phenyl)pyridin-2(1H)-one (100 mg, 206 μmol, 1.00 eq) at 20 °C, the mixture was stirred at 20 °C for 12 h. LCMS showed 3-(1-methyl-6-(1-(piperidin-4-ylmethyl)piperidin-4-yl)-1H-indazol-3-yl)piperidine-2,6-dione was consumed and the desired target MS was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to give the title compound (Compound 315) (5.50 mg, 6.27 μmol, 3.05% yield, 94.5% purity) as a white solid. LCMS (ES⁺): m/z 829.4 [M+H]⁺

### Example 373. Synthesis of 3-[6-[4-[2-[4-[4-[[5-fluoro-4-[3-(2-oxo-1-pyridyl)phenyl]pyrimidin-2-yl]amino]piperidine-1-carbonyl]-1-piperidyl]ethyl]-1-piperidyl]-2-oxo-1,3-benzoxazol-3-yl]piperidine-2,6-dione (Compound 330)

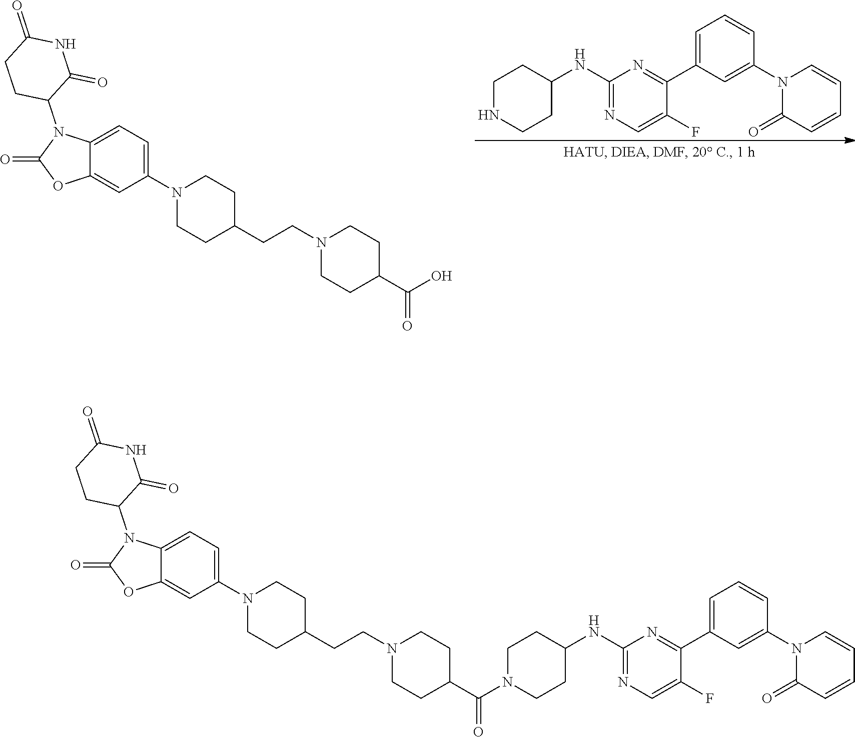 [SMILES: O=C1CCC(n2c(=O)oc3cc(N4CCC(CCN5CCC(C(=O)O)CC5)CC4)ccc32)C(=O)N1 ; O=c1ccccn1-c1cccc(-c2nc(NC3CCNCC3)ncc2F)c1 ; O=C1CCC(n2c(=O)oc3cc(N4CCC(CCN5CCC(C(=O)N6CCC(Nc7ncc(F)c(-c8cccc(-n9ccccc9=O)c8)n7)CC6)CC5)CC4)ccc32)C(=O)N1]

Synthesis of 3-[6-[4-[2-[4-[4-[[5-fluoro-4-[3-(2-oxo-1-pyridyl)phenyl]pyrimidin-2-yl]amino]piperidine-1-carbonyl]-1-piperidyl]ethyl]-1-piperidyl]-2-oxo-1,3-benzoxazol-3-yl]piperidine-2,6-dione (Compound 330). To a solution of 1-[2-[1-[3-(2,6-dioxo-3-piperidyl)-2-oxo-1,3-benzoxazol-6-yl]-4-piperidyl]ethyl]piperidine-4-carboxylic acid (36.0 mg, 74.3 mol, 1.00 eq) in DMF (2 mL) were added DIEA (48.2 mg, 373 μmol, 64.8 μL, 5.00 eq) and HATU (31.1 mg, 81.7 μmol, 1.10 eq) at 20 °C, the mixture was stirred at 20 °C for 0.5 h. Then 1-[3-[5-fluoro-2-(4-piperidylamino)pyrimidin-4-yl]phenyl]pyridin-2-one (35.6 mg, 74.3 μmol, 1.00 eq, TFA) was added at 20 °C, the mixture was stirred at 20 °C for 0.5 h. LCMS showed that 1-[2-[1-[3-(2,6-dioxo-3-piperidyl)-2-oxo-1,3-benzoxazol-6-yl]-4-piperidyl]ethyl]piperidine-4-carboxylic acid was consumed completely and the desired mass was detected. To the mixture was added H₂O (3 mL) and the mixture was extracted with EtOAc (3 mL*3). The combined organic layers were washed with brine (3 mL*3), dried over Na₂SO₄, filtered and the filtrate was concentrated under reduced pressure to give the crude product. The crude product was purified by neutral prep-HPLC (column: Waters Xbridge BEH C18 100*30 mm 5 um; mobile phase: [H₂O (10 mM NH₄HCO₃)-ACN]; gradient: 23%-54% B over 8.0 min) to give the title compound (Compound 330) (6.40 mg, 7.43 μmol, 10.0% yield, 96.6% purity) as a yellow solid. LCMS (ES⁺): m/z 832.4 [M+H]⁺

### Example 374. Synthesis of 3-(6-(1-((1-(1-(4-((5-fluoro-4-(3-(2-oxopyridin-1(2H)-yl)phenyl)pyrimidin-2-yl)amino)piperidin-1-yl)-1-oxopropan-2-yl)piperidin-4-yl)methyl)piperidin-4-yl)-2-oxobenzo[d]oxazol-3 (2H)-yl)piperidine-2,6-dione (Compound 370)

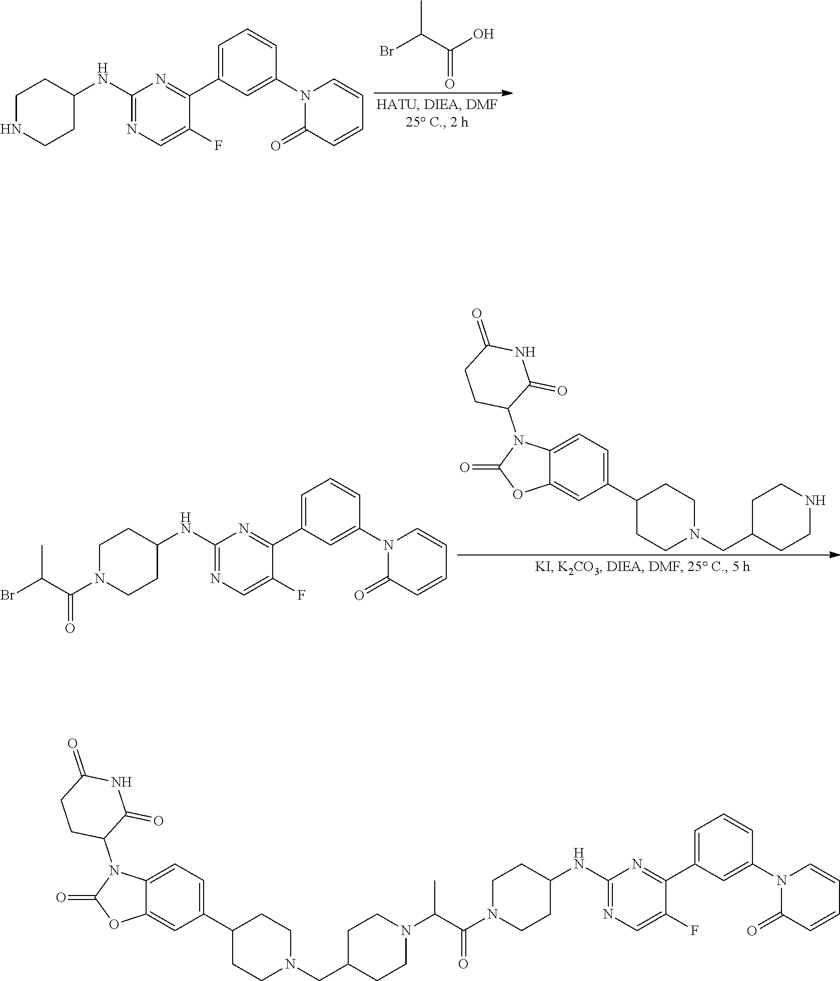

Synthesis of 1-(3-(2-((1-(2-bromopropanoyl)piperidin-4-yl)amino)-5-fluoropyrimidin-4-yl)phenyl)pyridin-2(1H)-one. To a solution of 1-[3-[5-fluoro-2-(4-piperidylamino)pyrimidin-4-yl]phenyl]pyridin-2-one (500 mg, 1.37 mmol, 1.00 eq) in DMF (3 mL) was added 2-bromopropanoic acid (209 mg, 1.37 mmol, 123 μL, 1.00 eq), HATU (780 mg, 2.05 mmol, 1.50 eq) and DIEA (353 mg, 2.74 mmol, 476 μL, 2.00 eq) at 25 °C. The mixture was stirred at 25 °C for 2 h. LC-MS showed the desired mass was detected. The reaction mixture was diluted with water 2 mL and extracted with EtOAc (5 mL*3), filtered and concentrated under reduced pressure. The residue was purified by prep-TLC (SiO₂, DCM:THF=1:0 to 1:1) to give the title compound (230 mg, 279 μmol, 20.4% yield, 60.7% purity) as a yellow solid. LCMS (ES⁺): m/z 500.2 [M+H]⁺.

Synthesis of 3-(6-(1-((1-(1-(4-((5-fluoro-4-(3-(2-oxopyridin-1(2H)-yl)phenyl)pyrimidin-2-yl)amino)piperidin-1-yl)-1-oxopropan-2-yl)piperidin-4-yl)methyl)piperidin-4-yl)-2-oxobenzo[d]oxazol-3 (2H)-yl)piperidine-2,6-dione (Compound 370). To a solution of 1-(3-(2-((1-(2-bromopropanoyl)piperidin-4-yl)amino)-5-fluoropyrimidin-4-yl)phenyl)pyridin-2(1H)-one (220 mg, 439 μmol, 1.00 eq) in DMF (3 mL) was added 3-(2-oxo-6-(1-(piperidin-4-ylmethyl)piperidin-4-yl)benzo[d]oxazol-3 (2H)-yl)piperidine-2,6-dione (187 mg, 439 μmol, 1.00 eq), DIEA (113 mg, 879 μmol, 153 μL, 2.00 eq), KI (72.9 mg, 439 μmol, 1.00 eq) and K₂CO₃ (121 mg, 879 μmol, 2.00 eq) at 25 °C. The mixture was stirred at 25 °C for 5 h. LC-MS showed the desired mass was detected. The reaction mixture was filtered to give a residue. The residue was purified by prep-HPLC (column: Phenomenex Luna C18 75*30 mm*3 um; mobile phase: [H₂O (0.1% TFA)-ACN]; gradient: 5%-35% B over 8.0 min). The residue was purified by prep-HPLC (column: Waters Xbridge BEH C18 100*25 mm*10 um; mobile phase: [H₂O (10 mM NH₄HCO₃)-ACN]; gradient: 25%-60% B over 8.0 min) to give the title compound (Compound 370) (22.0 mg, 26.0 μmol, 5.91% yield, 100% purity) as a white solid. LCMS (ES⁺): m/z 846.4 [M+H]⁺.

### Example 375. Synthesis of 3-(6-(4-((1-(2-(4-((5-fluoro-4-(3-(2-oxopyridin-1(2H)-yl)phenyl)pyrimidin-2-yl)amino)piperidin-1-yl)-2-oxoethyl)piperidin-4-yl)methyl)piperazin-1-yl)-2-oxobenzo[d]oxazol-3 (2H)-yl)piperidine-2,6-dione (Compound 340)

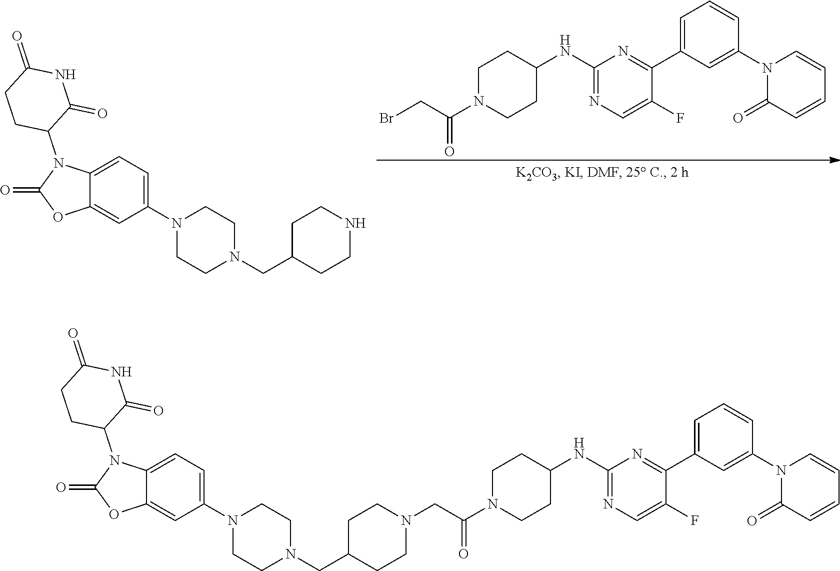 [SMILES: O=C1CCC(n2c(=O)oc3cc(N4CCN(CC5CCNCC5)CC4)ccc32)C(=O)N1 ; O=C(CBr)N1CCC(Nc2ncc(F)c(-c3cccc(-n4ccccc4=O)c3)n2)CC1 ; O=C1CCC(n2c(=O)oc3cc(N4CCN(CC5CCN(CC(=O)N6CCC(Nc7ncc(F)c(-c8cccc(-n9ccccc9=O)c8)n7)CC6)CC5)CC4)ccc32)C(=O)N1]

Synthesis of 3-(6-(4-((1-(2-(4-((5-fluoro-4-(3-(2-oxopyridin-1(2H)-yl)phenyl)pyrimidin-2-yl)amino)piperidin-1-yl)-2-oxoethyl)piperidin-4-yl)methyl)piperazin-1-yl)-2-oxobenzo[d]oxazol-3 (2H)-yl)piperidine-2,6-dione (Compound 340). To a solution of 3-(2-oxo-6-(4-(piperidin-4-ylmethyl)piperazin-1-yl)benzo[d]oxazol-3 (2H)-yl)piperidine-2,6-dione (120 mg, 280 μmol, 1.00 eq) in DMF (2.00 mL) was added K₂CO₃ (116 mg, 842 μmol, 3.00 eq), 1-[3-[2-[[1-(2-bromoacetyl)-4-piperidyl]amino]-5-fluoro-pyrimidin-4-yl]phenyl]pyridin-2-one (136 mg, 280 μmol, 1.00 eq) and KI (46.6 mg, 280 μmol, 1.00 eq) at 25 °C. The mixture was stirred at 25 °C for 2 h. LC-MS showed 3-(2-oxo-6-(4-(piperidin-4-ylmethyl)piperazin-1-yl)benzo[d]oxazol-3 (2H)-yl)piperidine-2,6-dione was consumed completely and the desired mass was detected. The reaction mixture was concentrated under reduced pressure. The residue was purified by prep-HPLC (column: Phenomenex luna C18 100*40 mm*3 um; mobile phase: [H₂O (0.1% TFA)-ACN]; gradient: 1%-30% B over 8.0 min) to give the title compound (Compound 340) (36.2 mg, 36.9 μmol, 13.1% yield, 96.7% purity, TFA) as an off white solid. LCMS (ES⁺): m/z 833.4 [M+H]⁺.

### Example 376. Synthesis of 3-((3-fluoro-4-(4-((1-(2-(4-((5-fluoro-4-(3-(2-oxopyridin-1(2H)-yl)phenyl)pyrimidin-2-yl)amino)piperidin-1-yl)-2-oxoethyl)piperidin-4-yl)methyl)piperazin-1-yl)phenyl)amino)piperidine-2,6-dione (Compound 341)

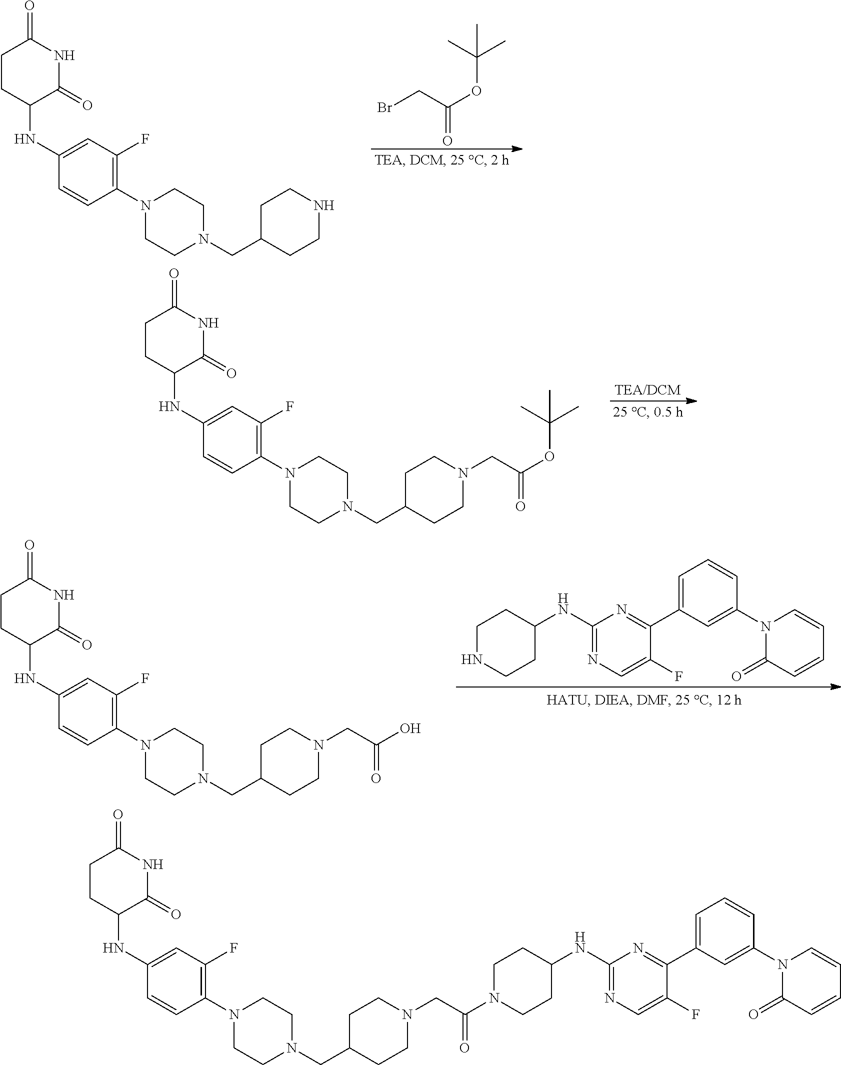

Synthesis of tert-butyl 2-(4-((4-(4-((2,6-dioxopiperidin-3-yl)amino)-2-fluorophenyl)piperazin-1-yl)methyl)piperidin-1-yl)acetate. To a solution of 3-((3-fluoro-4-(4-(piperidin-4-ylmethyl)piperazin-1-yl)phenyl)amino)piperidine-2,6-dione (1.00 g, 1.93 mmol, 1.00 eq, TFA) in DCM (10 mL) was added TEA (977 mg, 9.66 mmol, 1.34 mL, 5.00 eq) and tert-butyl 2-bromoacetate (753 mg, 3.86 mmol, 570.63 μL, 2.00 eq) at 25 °C. The mixture was stirred at 25 °C for 2 h. LC-MS showed desired mass was detected. The reaction mixture was diluted with H₂O 30 mL and extracted with EtOAc (30 mL*3). The combined organic layers were concentrated under reduced pressure to give the title compound (1.00 g, crude) as a brown oil. LCMS (ES⁺): m/z 518.3 [M+H]⁺.

Synthesis of 2-(4-((4-(4-((2,6-dioxopiperidin-3-yl)amino)-2-fluorophenyl)piperazin-1-yl)methyl)piperidin-1-yl)acetic acid. To a solution of tert-butyl 2-(4-((4-(4-((2,6-dioxopiperidin-3-yl)amino)-2-fluorophenyl)piperazin-1-yl)methyl)piperidin-1-yl)acetate (950 mg, 1.84 mmol, 1.00 eq) in DCM (10 mL) was added TFA (15.35 g, 134.62 mmol, 10 mL, 73.35 eq) at 25 °C. The mixture was stirred at 25 °C for 0.5 h. LC-MS showed desired mass was detected. The reaction mixture was concentrated under reduced pressure to give a residue. The residue was purified by prep-HPLC (column: 3_Phenomenex Luna C18 75*30 mm*3 um; mobile phase: [H₂O (0.1% TFA)-ACN]; gradient: 1%-20% B over 8.0 min) to give the title compound (400 mg, 678 μmol, 36.9% yield, 97.6% purity, TFA) as a brown oil. LCMS (ES⁺): m/z 462.2 [M+H]⁺.

Synthesis of 3-((3-fluoro-4-(4-((1-(2-(4-((5-fluoro-4-(3-(2-oxopyridin-1(2H)-yl)phenyl)pyrimidin-2-yl)amino)piperidin-1-yl)-2-oxoethyl)piperidin-4-yl)methyl)piperazin-1-yl)phenyl)amino)piperidine-2,6-dione (Compound 341). To a solution of 2-(4-((4-(4-((2,6-dioxopiperidin-3-yl)amino)-2-fluorophenyl)piperazin-1-yl)methyl)piperidin-1-yl)acetic acid (100 mg, 216 μmol, 1.00 eq, TFA) in DMF (5 mL) was added 1-(3-(5-fluoro-2-(piperidin-4-ylamino)pyrimidin-4-yl)phenyl)pyridin-2(1H)-one (79.1 mg, 216 μmol, 1.00 eq), DIEA (84.0 mg, 650 μmol, 113 μL, 3.00 eq) and HATU (123 mg, 325 μmol, 1.50 eq) at 25 °C. The mixture was stirred at 25 °C for 12 h. LC-MS showed desired mass was detected. The reaction mixture was concentrated under reduced pressure. The residue was purified by prep-HPLC (column: 3_Phenomenex Luna C18 75*30 mm*3 um; mobile phase: [H₂O (0.1% TFA)-ACN]; gradient: 1%-30% B over 8.0 min) to give the title compound (Compound 341) (47.9 mg, 50.25 μmol, 23.19% yield, 96.82% purity, TFA) as a gray solid. LCMS (ES⁺): m/z 809.4 [M+H]⁺.

### Example 377. Synthesis of 3-(6-(4-((4-(2-(4-((5-fluoro-4-(3-(2-oxopyridin-1(2H)-yl)phenyl)pyrimidin-2-yl)amino)piperidin-1-yl)-2-oxoethyl)piperazin-1-yl)methyl)piperidin-1-yl)-2-oxobenzo[d]oxazol-3 (2H)-yl)piperidine-2,6-dione (Compound 378)

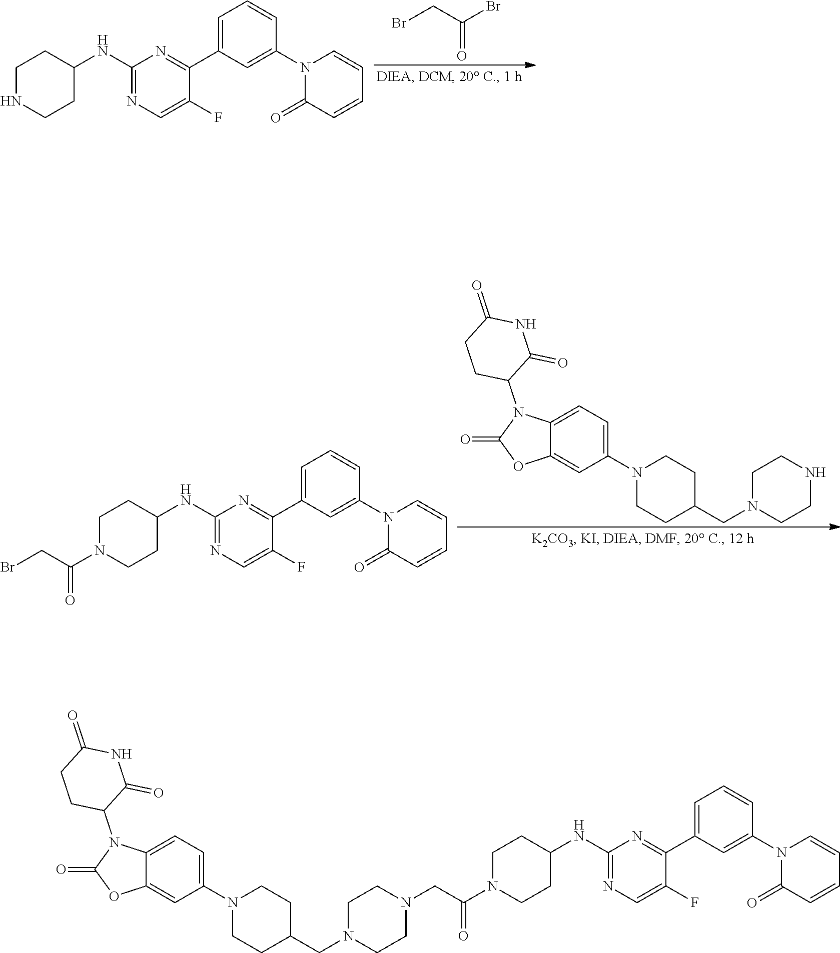 [SMILES: O=c1ccccn1-c1cccc(-c2nc(NC3CCNCC3)ncc2F)c1 ; O=C(Br)CBr ; O=C(CBr)N1CCC(Nc2ncc(F)c(-c3cccc(-n4ccccc4=O)c3)n2)CC1 ; O=C1CCC(n2c(=O)oc3cc(N4CCC(CN5CCNCC5)CC4)ccc32)C(=O)N1 ; O=C1CCC(n2c(=O)oc3cc(N4CCC(CN5CCN(CC(=O)N6CCC(Nc7ncc(F)c(-c8cccc(-n9ccccc9=O)c8)n7)CC6)CC5)CC4)ccc32)C(=O)N1]

Synthesis of 1-(3-(2-((1-(2-bromoacetyl)piperidin-4-yl)amino)-5-fluoropyrimidin-4-yl)phenyl)pyridin-2(1H)-one. A mixture of 1-(3-(5-fluoro-2-(piperidin-4-ylamino)pyrimidin-4-yl)phenyl)pyridin-2(1H)-one (400 mg, 1.09 mmol, 1.00 eq, HCl), DIEA (424 mg, 3.28 mmol, 572 μL, 3.00 eq) and 2-bromoacetyl bromide (220 mg, 1.09 mmol, 95.3 L, 1.00 eq) in DCM (10 mL) at 20 °C was degassed and purged with N₂ for 3 times, and then the mixture was stirred at 20 °C for 1 h under N₂ atmosphere. LC-MS showed the desired mass was detected. The reaction mixture was filtered. The residue was purified by column chromatography (SiO₂, Petroleum ether:Ethyl acetate=1:0 to 0:1) to give the title compound (120 mg, 175 μmol, 16.0% yield, 71.0% purity) as a yellow solid. LCMS (ES⁺): m/z 486.1 [M+H]⁺.

Synthesis of 3-(6-(4-((4-(2-(4-((5-fluoro-4-(3-(2-oxopyridin-1(2H)-yl)phenyl)pyrimidin-2-yl)amino)piperidin-1-yl)-2-oxoethyl)piperazin-1-yl)methyl)piperidin-1-yl)-2-oxobenzo[d]oxazol-3 (2H)-yl)piperidine-2,6-dione (Compound 378). To a solution of 1-(3-(2-((1-(2-bromoacetyl)piperidin-4-yl)amino)-5-fluoropyrimidin-4-yl)phenyl)pyridin-2(1H)-one (56.8 mg, 116 μmol, 1.00 eq) and 3-(2-oxo-6-(4-(piperazin-1-ylmethyl)piperidin-1-yl)benzo[d]oxazol-3 (2H)-yl)piperidine-2,6-dione (50.0 mg, 116 μmol, 1.00 eq) in DMF (2 mL) was added DIEA (30.2 mg, 233 μmol, 40.7 μL, 2.00 eq), K₂CO₃ (32.3 mg, 233 μmol, 2.00 eq) and KI (38.8 mg, 233 μmol, 2.00 eq) at 20 °C. The mixture was stirred at 20 °C for 12 h. LC-MS showed the desired mass was detected. The reaction mixture was filtered and concentrated under reduced pressure. The residue was purified by prep-HPLC (column: 3_Phenomenex Luna C18 75*30 mm*3 um; mobile phase: [H₂O (0.2% FA)-ACN]; gradient: 1%-30% B over 8.0 min) to give the title compound (Compound 378) (15.1 mg, 17.81 μmol, 15.2% yield, 98.2% purity, 0.3 FA) as a purple solid. LCMS (ES⁺): m/z 833.3 [M+H]⁺

### Example 378. Synthesis of 3-(6-(1-(2-(4-(4-((5-fluoro-4-(3-(2-oxopyridin-1(2H)-yl)phenyl)pyrimidin-2-yl)amino)piperidine-1-carbonyl)-4-hydroxypiperidin-1-yl)-2-oxoethyl)piperidin-4-yl)-2-oxobenzo[d]oxazol-3 (2H)-yl)piperidine-2,6-dione (Compound 331)

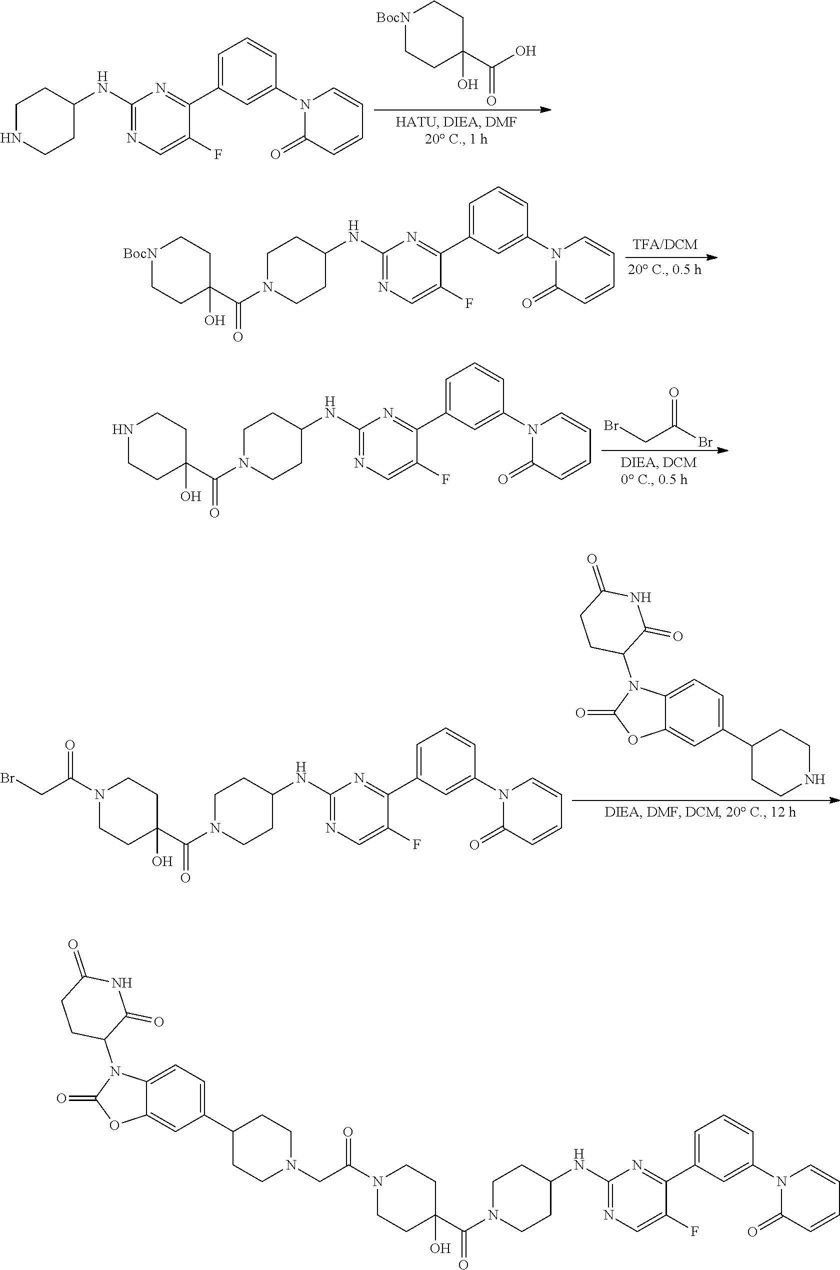

Synthesis of tert-butyl 4-(4-((5-fluoro-4-(3-(2-oxopyridin-1(2H)-yl)phenyl)pyrimidin-2-yl)amino)piperidine-1-carbonyl)-4-hydroxypiperidine-1-carboxylate. To a solution of 1-(tert-butoxycarbonyl)-4-hydroxypiperidine-4-carboxylic acid (732 mg, 2.99 mmol, 1.00 eq) in DMF (30 mL) were added HATU (2.27 g, 5.97 mmol, 2.00 eq), DIEA (1.16 g, 8.96 mmol, 1.56 mL, 3.00 eq) and 1-(3-(5-fluoro-2-(piperidin-4-ylamino)pyrimidin-4-yl)phenyl)pyridin-2(1H)-one (1.20 g, 2.99 mmol, 1.00 eq, HCl) at 20 °C. The mixture was stirred at 20 °C for 1 h. LC-MS showed 1-(3-(5-fluoro-2-(piperidin-4-ylamino)pyrimidin-4-yl)phenyl)pyridin-2(1H)-one was consumed completely and the desired mass was detected. Water (30 mL) was added to the mixture at 20 °C. Then the reaction mixture was extracted with EtOAc (20 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=3:1 to 1:1) to give the title compound (1.00 g, 1.69 mmol, 56.5% yield) as a white solid. LCMS (ES⁺): m/z 593.3 [M+H]⁺

Synthesis of 1-(3-(5-fluoro-2-((1-(4-hydroxypiperidine-4-carbonyl)piperidin-4-yl)amino)pyrimidin-4-yl)phenyl)pyridin-2(1H)-one. To a solution of tert-butyl 4-(4-((5-fluoro-4-(3-(2-oxopyridin-1(2H)-yl)phenyl)pyrimidin-2-yl)amino)piperidine-1-carbonyl)-4-hydroxypiperidine-1-carboxylate (200 mg, 337 μmol, 1.00 eq) in DCM (3 mL) was added TFA (1.54 g, 13.5 mmol, 1 mL, 39.9 eq) at 20 °C. The mixture was stirred at 20 °C for 0.5 h. LC-MS showed tert-butyl 4-(4-((5-fluoro-4-(3-(2-oxopyridin-1(2H)-yl)phenyl)pyrimidin-2-yl)amino)piperidine-1-carbonyl)-4-hydroxypiperidine-1-carboxylate was consumed completely and the desired mass was detected. The reaction mixture was concentrated under reduced pressure to give the title compound (204 mg, crude, TFA) as a brown oil. LCMS (ES⁺): m/z 493.3 [M+H]⁺.

Synthesis of 1-(3-(2-((1-(1-(2-bromoacetyl)-4-hydroxypiperidine-4-carbonyl)piperidin-4-yl)amino)-5-fluoropyrimidin-4-yl)phenyl)pyridin-2(1H)-one. To a solution of 1-(3-(5-fluoro-2-((1-(4-hydroxypiperidine-4-carbonyl)piperidin-4-yl)amino)pyrimidin-4-yl)phenyl)pyridin-2(1H)-one (204 mg, 336 μmol, 1.00 eq, TFA) in DCM (3 mL) was added DIEA (130 mg, 1.01 mmol, 176 μL, 3.00 eq) and 2-bromoacetyl bromide (136 mg, 673 μmol, 58.6 μL, 2.00 eq) at 0 °C. The mixture was stirred at 0 °C for 0.5 h. LC-MS showed 1-(3-(5-fluoro-2-((1-(4-hydroxypiperidine-4-carbonyl)piperidin-4-yl)amino)pyrimidin-4-yl)phenyl)pyridin-2(1H)-one was consumed completely and the desired mass was detected. The mixture was used into the next step without further work up. The title compound (206 mg, 336 μmol, 99.8% yield) was obtained in DCM (3 mL) as a brown liquid. LCMS (ES⁺): m/z 613.4 [M+H]⁺.

Synthesis of 3-(6-(1-(2-(4-(4-((5-fluoro-4-(3-(2-oxopyridin-1(2H)-yl)phenyl)pyrimidin-2-yl)amino)piperidine-1-carbonyl)-4-hydroxypiperidin-1-yl)-2-oxoethyl)piperidin-4-yl)-2-oxobenzo[d]oxazol-3 (2H)-yl)piperidine-2,6-dione (Compound 331). To a solution of 3-(2-oxo-6-(piperidin-4-yl)benzo[d]oxazol-3 (2H)-yl)piperidine-2,6-dione (149 mg, 336 μmol, 1.00 eq, TFA) in DMF (4 mL) was added DIEA (130 mg, 1.01 mmol, 175 μL, 3.00 eq) at 20 °C. Then a solution of 1-(3-(2-((1-(1-(2-bromoacetyl)-4-hydroxypiperidine-4-carbonyl)piperidin-4-yl)amino)-5-fluoropyrimidin-4-yl)phenyl)pyridin-2(1H)-one (206 mg, 336 μmol, 1.00 eq) in DCM (3 mL) was added to the mixture at 20 °C. The mixture was stirred at 20 °C for 12 h. LC-MS showed 1-(3-(2-((1-(1-(2-bromoacetyl)-4-hydroxypiperidine-4-carbonyl)piperidin-4-yl)amino)-5-fluoropyrimidin-4-yl)phenyl)pyridin-2(1H)-one was consumed completely and the desired mass was detected. The reaction mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 5%-40% B over 8.0 min) to give the title compound (Compound 331) (30.7 mg, 35.2 μmol, 10.5% yield, 98.9% purity) as a white solid. LCMS (ES⁺): m/z 862.4 [M+H]⁺

### Example 379. Synthesis of 1-(6-(1-(2-(4-(4-((5-fluoro-4-(3-(2-oxopyridin-1(2H)-yl)phenyl)pyrimidin-2-yl)amino)piperidine-1-carbonyl)-4-hydroxypiperidin-1-yl)-2-oxoethyl)piperidin-4-yl)-1-methyl-1H-indazol-3-yl)dihydropyrimidine-2,4(1H,3H)-dione (Compound 346)

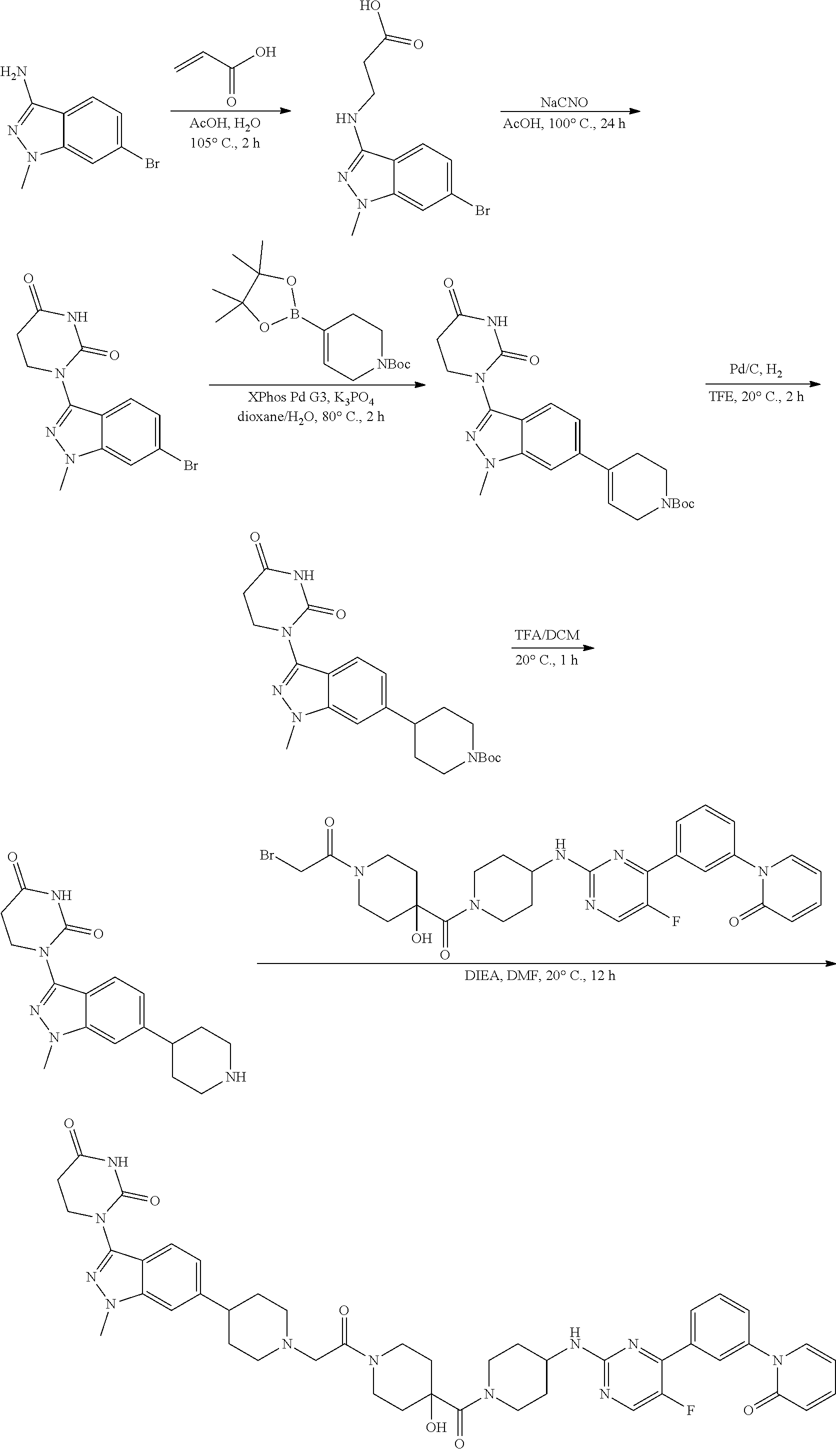

Synthesis of 3-((6-bromo-1-methyl-1H-indazol-3-yl)amino)propanoic acid. To a solution of 6-bromo-1-methyl-1H-indazol-3-amine (8.50 g, 37.6 mmol, 1.00 eq) in H₂O (85 mL) were added AcOH (5.35 g, 89.1 mmol, 5.10 mL, 2.37 eq) and acrylic acid (2.71 g, 37.6 mmol, 2.58 mL, 1.00 eq) at 20 °C under N₂ atmosphere. The mixture was stirred at 105 °C for 2 h. LCMS showed the desired mass was detected. NaOH (1 M) was added to the mixture at 20 °C to adjust pH to 8-9. Then the reaction mixture was extracted with MTBE (40 mL 3). The aqueous layer was separated and it was acidified with HCl (1 M), solid was precipitated. The solid was filtered and washed with water, dried under reduced pressure to give the title compound (2.00 g, crude) as a yellow solid. LCMS (ES⁺): m/z 298.0 [M+H]⁺.

Synthesis of 1-(6-bromo-1-methyl-1H-indazol-3-yl)dihydropyrimidine-2,4(1H,3H)-dione. To a solution of 3-((6-bromo-1-methyl-1H-indazol-3-yl)amino)propanoic acid (500 mg, 1.68 mmol, 1.00 eq) in AcOH (10 mL) was added sodium; cyanate (145 mg, 2.23 mmol, 1.33 eq) at 20 °C. The mixture was stirred at 100 °C for 12 h. LCMS showed 3-((6-bromo-1-methyl-1H-indazol-3-yl)amino)propanoic acid was remaining and the desired mass was detected. The mixture was stirred at 100 °C for another 12 h. LCMS showed 3-((6-bromo-1-methyl-1H-indazol-3-yl)amino)propanoic acid was consumed completely and the desired mass was detected. Water (15 mL) was added to the mixture at 20 °C. Then the reaction mixture was extracted with DCM (10 mL*3). The organic layers were dried over anhydrous sodium sulfate, filtered and the filtrate was concentrated under reduced pressure to give a residue. The residue was purified by prep-TLC (SiO₂, Petroleum ether:Ethyl acetate=0:1) to give the title compound (280 mg, 866 μmol, 17.2% yield) as a colorless oil. LCMS (ES⁺): m/z 323.0 [M+H]⁺.

Synthesis of tert-butyl 4-(3-(2,4-dioxotetrahydropyrimidin-1(2H)-yl)-1-methyl-1H-indazol-6-yl)-3,6-dihydropyridine-1(2H)-carboxylate. To a solution of 1-(6-bromo-1-methyl-1H-indazol-3-yl)dihydropyrimidine-2,4(1H,3H)-dione (380 mg, 1.18 mmol, 1.00 eq) and tert-butyl 4-(4,4,5,5-tetramethyl-1,3,2-dioxaborolan-2-yl)-3,6-dihydro-2H-pyridine-1-carboxylate (545 mg, 1.76 mmol, 1.50 eq) in dioxane (8 mL) and H₂O (1.6 mL) were added K₃PO₄ (749 mg, 3.53 mmol, 3.00 eq) and XPhos Pd G3 (99.4 mg, 118 μmol, 0.100 eq) at 20 °C under N₂ atmosphere. The mixture was stirred at 80 °C for 2 h under N₂. LCMS showed the desired mass was detected. Water (8 mL) was added to the mixture at 20 °C. Then the reaction mixture was extracted with EtOAc (5 mL*3). The organic layers were dried over anhydrous sodium sulfate, filtered and the filtrate was concentrated under reduced pressure to give a residue. The residue was purified by prep-TLC (SiO₂, Petroleum ether:Ethyl acetate=0:1) to give the title compound (380 mg, 893 umol, 75.9% yield) as a yellow oil. LCMS (ES⁺): m/z 426.2 [M+H]⁺.

Synthesis of tert-butyl 4-(3-(2,4-dioxotetrahydropyrimidin-1(2H)-yl)-1-methyl-1H-indazol-6-yl)piperidine-1-carboxylate. 10% Pd/C (400 mg, 50.0% purity) was added to TFE (10 mL) at 20 °C under N₂ atmosphere, then tert-butyl 4-(3-(2,4-dioxotetrahydropyrimidin-1(2H)-yl)-1-methyl-1H-indazol-6-yl)-3,6-dihydropyridine-1(2H)-carboxylate (380 mg, 893 μmol, 1.00 eq) was added to the mixture at 20 °C, the suspension was degassed and purged with H₂ for three times. The mixture was stirred at 20 °C for 2 h under H₂ (15 Psi) atmosphere. LCMS showed tert-butyl 4-(3-(2,4-dioxotetrahydropyrimidin-1(2H)-yl)-1-methyl-1H-indazol-6-yl)-3,6-dihydropyridine-1(2H)-carboxylate was consumed completely and the desired mass was detected. The organic layers were filtered and the filtrate was concentrated under reduced pressure to give the title compound (380 mg, crude) as a yellow oil. LCMS (ES⁺): m/z 372.2 [M+H−56]⁺.

Synthesis of 1-(1-methyl-6-(piperidin-4-yl)-1H-indazol-3-yl)dihydropyrimidine-2,4(1H,3H)-dione. To a solution of tert-butyl 4-(3-(2,4-dioxotetrahydropyrimidin-1(2H)-yl)-1-methyl-1H-indazol-6-yl)piperidine-1-carboxylate (150 mg, 351 μmol, 1.00 eq) in DCM (3 mL) was added TFA (1 mL) at 20 °C. The mixture was stirred at 20 °C for 1 h. LCMS showed tert-butyl 4-(3-(2,4-dioxotetrahydropyrimidin-1(2H)-yl)-1-methyl-1H-indazol-6-yl)piperidine-1-carboxylate was consumed completely and the desired mass was detected. The reaction mixture was concentrated under reduced pressure to give the title compound (150 mg, crude, TFA) as a yellow oil. LCMS (ES⁺): m/z 328.2 [M+H]⁺.

Synthesis of 1-(6-(1-(2-(4-(4-((5-fluoro-4-(3-(2-oxopyridin-1(2H)-yl)phenyl)pyrimidin-2-yl)amino)piperidine-1-carbonyl)-4-hydroxypiperidin-1-yl)-2-oxoethyl)piperidin-4-yl)-1-methyl-1H-indazol-3-yl)dihydropyrimidine-2,4(1H,3H)-dione (Compound 346). To a solution of 1-(1-methyl-6-(piperidin-4-yl)-1H-indazol-3-yl)dihydropyrimidine-2,4(1H,3H)-dione (145 mg, 329 μmol, 1.00 eq, TFA) in DMF (3 mL) were added DIEA (127 mg, 985 μmol, 172 μL, 3.00 eq) and 1-[3-[2-[[1-[1-(2-bromoacetyl)-4-hydroxy-piperidine-4-carbonyl]-4-piperidyl]amino]-5-fluoro-pyrimidin-4-yl]phenyl]pyridin-2-one (202 mg, 329 μmol, 1.00 eq) at 20 °C. The mixture was stirred at 20 °C for 12 h. LCMS showed 1-(1-methyl-6-(piperidin-4-yl)-1H-indazol-3-yl)dihydropyrimidine-2,4(1H,3H)-dione was consumed completely and the desired mass was detected. The reaction was filtered and the filtrate was concentrated under reduced pressure to give a residue. The residue was purified by prep-HPLC (column: Waters Xbridge BEH C18 100*30 mm 5 um; mobile phase: [H₂O (10 mM NH₄HCO₃)-ACN]; gradient: 23%-48% B over 8.0 min) to give the title compound (Compound 346) (11.3 mg, 13.0 μmol, 3.94% yield, 98.6% purity) as a white solid. LCMS (ES⁺): m/z 860.4 [M+H]⁺

### Example 380. Synthesis of 3-(6-(4-((4-(2-(4-((5-fluoro-4-(3-(2-oxopyridin-1(2H)-yl)phenyl)pyrimidin-2-yl)amino)piperidin-1-yl)-2-oxoethyl)-4-hydroxypiperidin-1-yl)methyl)piperidin-1-yl)-2-oxobenzo[d]oxazol-3 (2H)-yl)piperidine-2,6-dione (Compound 380)

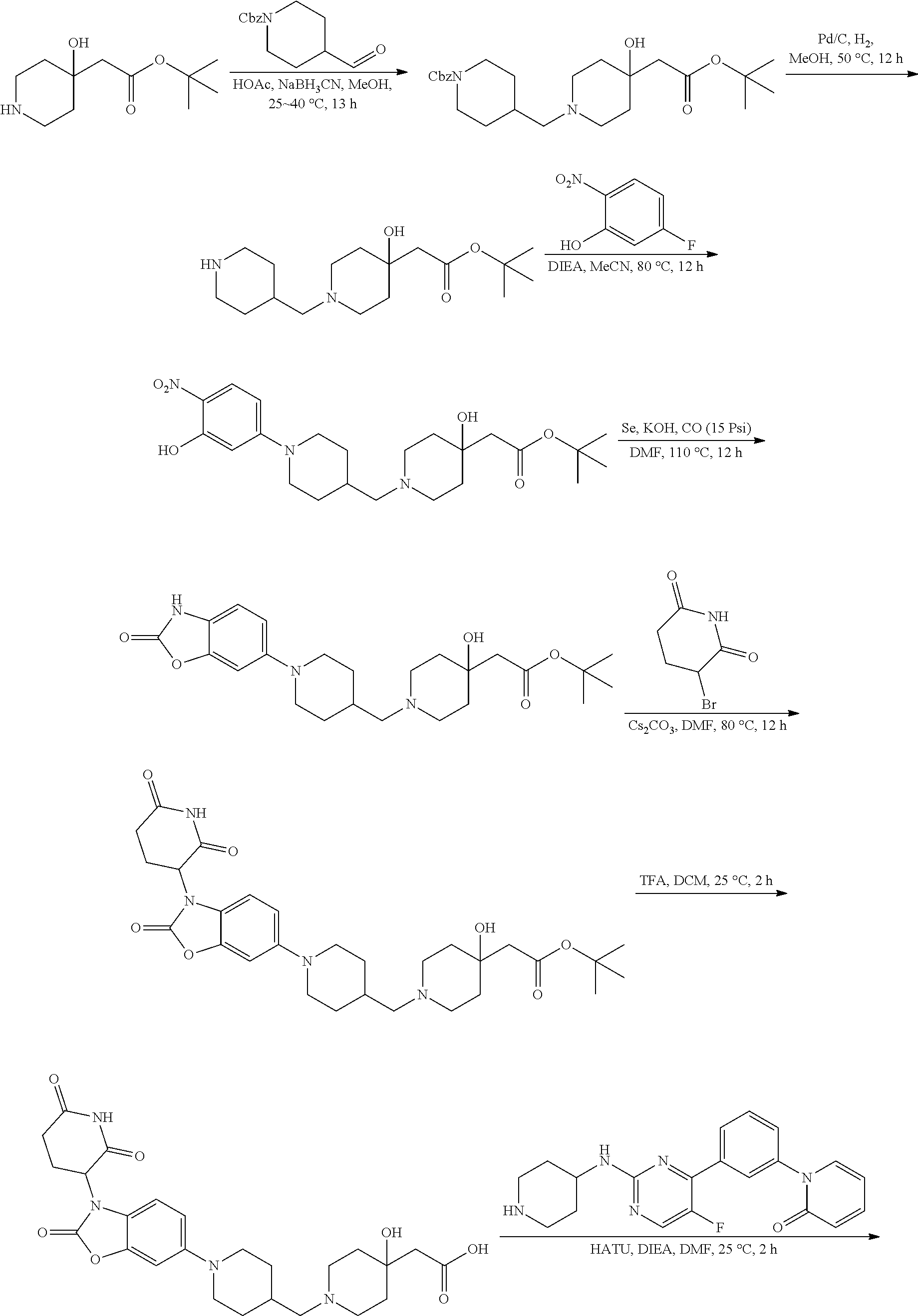

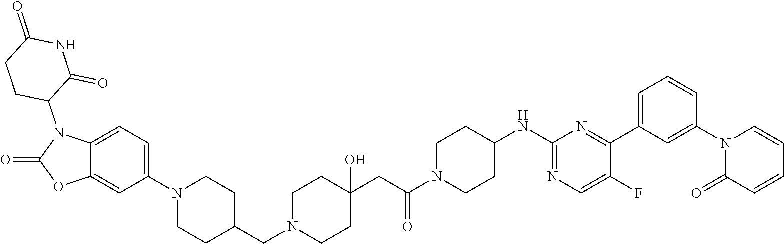 [SMILES: O=C1CCC(n2c(=O)oc3cc(N4CCC(CN5CCC(O)(CC(=O)N6CCC(Nc7ncc(F)c(-c8cccc(-n9ccccc9=O)c8)n7)CC6)CC5)CC4)ccc32)C(=O)N1]

Synthesis of benzyl 4-[[4-(2-tert-butoxy-2-oxo-ethyl)-4-hydroxy-1-piperidyl]methyl]piperidine-1-carboxylate. To a solution of tert-butyl 2-(4-hydroxy-4-piperidyl)acetate (5.00 g, 23.2 mmol, 1.00 eq) in MeOH (20 mL) was added AcOH (1.39 g, 23.2 mmol, 1.33 mL, 1.00 eq) and benzyl 4-formylpiperidine-1-carboxylate (17.2 g, 69.6 mmol, 3.00 eq) at 25 °C for 1 h, and add NaBH₃CN (7.30 g, 116 mmol, 5.00 eq) at 25 °C. The mixture was stirred at 40 °C for 12 h. LC-MS showed the desired mass was detected. The reaction diluted with H₂O 150 mL and combined organic layers were washed with EtOAc (200 mL*3), filtered and concentrated under reduced pressure. The residue was purified by column chromatography (SiO₂, Dichloromethane:Methanol=1:0 to 5:1) to give the title compound (20.0 g, 33.7 mmol, 72.5% yield, 75.2% purity) as a yellow oil. LCMS (ES⁺): m/z 447.3 [M+H]⁺.

Synthesis of tert-butyl 2-[4-hydroxy-1-(4-piperidylmethyl)-4-piperidyl]acetate. To a solution of benzyl 4-[[4-(2-tert-butoxy-2-oxo-ethyl)-4-hydroxy-1-piperidyl]methyl]piperidine-1-carboxylate (5.00 g, 11.2 mmol, 1.00 eq) in EtOH (10 mL) was added Pd/C (2.86 g, 13.4 mmol, 50.0% purity, 1.20 eq) at 25 °C under N₂ atmosphere. The suspension was degassed and purged with H₂ for 3 times. The mixture was stirred under H₂ (50 Psi) at 50 °C for 12 h. LC-MS showed the desired mass was detected. The reaction mixture was filtered and concentrated under reduced pressure o give the title compound (5.00 g, crude) as a brown oil. LCMS (ES⁺): m/z 313.3 [M+H]⁺.

Synthesis of tert-butyl 2-(4-hydroxy-1-((1-(3-hydroxy-4-nitrophenyl)piperidin-4-yl)methyl)piperidin-4-yl)acetate. To a solution of tert-butyl 2-(4-hydroxy-1-(piperidin-4-ylmethyl)piperidin-4-yl)acetate (3.00 g, 9.60 mmol, 1.00 eq) in MeCN (30 mL) was added DIEA (3.72 g, 28.8 mmol, 5.02 mL, 3.00 eq) and 5-fluoro-2-nitro-phenol (1.51 g, 9.60 mmol, 1.00 eq) at 25 °C. The mixture was stirred at 80 °C for 12 h. LC-MS showed tert-butyl 2-(4-hydroxy-1-(piperidin-4-ylmethyl)piperidin-4-yl)acetate was consumed completely and the desired mass was detected. The reaction diluted with H₂O 30 mL and combined organic layers were extracted with EtOAc (50 mL*3), and concentrated under reduced pressure. The residue was purified by column chromatography (SiO₂, Dichloromethane:Methanol=1:0 to 5:1) to give the title compound (3.00 g, 5.61 mmol, 58.4% yield, 84.1% purity) as a black solid. LCMS (ES⁺): m/z 450.3 [M+H]⁺.

Synthesis of tert-butyl 2-(4-hydroxy-1-((1-(2-oxo-2,3-dihydrobenzo[d]oxazol-6-yl)piperidin-4-yl)methyl)piperidin-4-yl)acetate. To a solution of tert-butyl 2-(4-hydroxy-1-((1-(3-hydroxy-4-nitrophenyl)piperidin-4-yl)methyl)piperidin-4-yl)acetate (3.00 g, 6.67 mmol, 1.00 eq) in DMF (20 mL) was added KOH (748 mg, 13.3 mmol, 2.00 eq) and Se (527 mg, 6.67 mmol, 1.00 eq) at 25 °C under N₂ atmosphere. The suspension was degassed and purged with CO (6.67 mmol, 1.00 eq) for 4 times. The mixture was stirred under CO (6.67 mmol, 1.00 eq) (15 Psi) at 110 °C for 12 h. LC-MS showed tert-butyl 2-(4-hydroxy-1-((1-(3-hydroxy-4-nitrophenyl)piperidin-4-yl)methyl)piperidin-4-yl)acetate was consumed completely and the desired mass was detected. The reaction diluted with H₂O 30 mL and the combined organic layers were extracted with EtOAc (70 mL*3), and concentrated under reduced pressure. The residue was purified by column chromatography (SiO₂, Dichloromethane:Methanol=1:0 to 5:1) to give the title compound (750 mg, 1.59 mmol, 23.8% yield, 94.5% purity) as a brown oil. LCMS (ES⁺): m/z 446.3 [M+H]⁺.

Synthesis of tert-butyl 2-(1-((1-(3-(2,6-dioxopiperidin-3-yl)-2-oxo-2,3-dihydrobenzo[d]oxazol-6-yl)piperidin-4-yl)methyl)-4-hydroxypiperidin-4-yl)acetate. To a solution of tert-butyl 2-(4-hydroxy-1-((1-(2-oxo-2,3-dihydrobenzo[d]oxazol-6-yl)piperidin-4-yl)methyl)piperidin-4-yl)acetate (400 mg, 897 μmol, 1.00 eq) in DMF (2 mL) was added Cs₂CO₃ (877 mg, 2.69 mmol, 3.00 eq) and 3-bromopiperidine-2,6-dione (861 mg, 4.49 mmol, 5.00 eq) at 25 °C. The mixture was stirred at 80 °C for 12 h. LC-MS showed tert-butyl 2-(4-hydroxy-1-((1-(2-oxo-2,3-dihydrobenzo[d]oxazol-6-yl)piperidin-4-yl)methyl)piperidin-4-yl)acetate remained and the desired mass was detected. The reaction mixture was filtered and concentrated under reduced pressure. The residue was purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min) to give the title compound (120 mg, 142 μmol, 15.8% yield, 66.0% purity) as a pink solid. LCMS (ES⁺): m/z 557.5 [M+H]⁺.

Synthesis of 2-(1-((1-(3-(2,6-dioxopiperidin-3-yl)-2-oxo-2,3-dihydrobenzo[d]oxazol-6-yl)piperidin-4-yl)methyl)-4-hydroxypiperidin-4-yl)acetic acid. To a solution of tert-butyl 2-(1-((1-(3-(2,6-dioxopiperidin-3-yl)-2-oxo-2,3-dihydrobenzo[d]oxazol-6-yl)piperidin-4-yl)methyl)-4-hydroxypiperidin-4-yl)acetate (80.0 mg, 143 μmol, 1.00 eq) in DCM (3 mL) was added TFA (1.54 g, 13.4 mmol, 1.00 mL, 93.6 eq) at 25 °C. The mixture was stirred at 25 °C for 2 h. LC-MS showed tert-butyl 2-(1-((1-(3-(2,6-dioxopiperidin-3-yl)-2-oxo-2,3-dihydrobenzo[d]oxazol-6-yl)piperidin-4-yl)methyl)-4-hydroxypiperidin-4-yl)acetate was consumed completely and the desired mass was detected. The reaction mixture was concentrated under reduced pressure. The residue was purified by prep-HPLC (column: Phenomenex Luna C18 100*30 mm*3 um; mobile phase: [H₂O (0.2% FA)-ACN]; gradient: 1%-5% B over 8.0 min) to give the title compound (35.0 mg, 69.9 μmol, 48.6% yield, 100% purity) as a white solid. LCMS (ES⁺): m/z 501.3 [M+H]⁺.

Synthesis of 3-(6-(4-((4-(2-(4-((5-fluoro-4-(3-(2-oxopyridin-1(2H)-yl)phenyl)pyrimidin-2-yl)amino)piperidin-1-yl)-2-oxoethyl)-4-hydroxypiperidin-1-yl)methyl)piperidin-1-yl)-2-oxobenzo[d]oxazol-3 (2H)-yl)piperidine-2,6-dione (Compound 380). To a solution of 2-(1-((1-(3-(2,6-dioxopiperidin-3-yl)-2-oxo-2,3-dihydrobenzo[d]oxazol-6-yl)piperidin-4-yl)methyl)-4-hydroxypiperidin-4-yl)acetic acid (30.0 mg, 59.9 μmol, 1.00 eq) in DMF (2 mL) was added HATU (22.7 mg, 59.9 μmol, 1.00 eq), 1-[3-[5-fluoro-2-(4-piperidylamino)pyrimidin-4-yl]phenyl]pyridin-2-one (21.9 mg, 59.9 μmol, 1.00 eq) and DIEA (23.2 mg, 179 μmol, 31.3 μL, 3.00 eq) at 25 °C. The mixture was stirred at 25 °C for 2 h. LC-MS showed 2-(1-((1-(3-(2,6-dioxopiperidin-3-yl)-2-oxo-2,3-dihydrobenzo[d]oxazol-6-yl)piperidin-4-yl)methyl)-4-hydroxypiperidin-4-yl)acetic acid was consumed completely and the desired mass was detected. The reaction mixture was concentrated under reduced pressure. The residue was purified by prep-HPLC (column: Waters Xbridge BEH C18 100*30 mm*10 um; mobile phase: [H₂O (10 mM NH₄HCO₃)-ACN]; gradient: 25%-55% B over 8.0 min) to give the title compound (Compound 380) (13.8 mg, 15.6 μmol, 26.0% yield, 95.8% purity) as a white solid. LCMS (ES⁺): m/z 848.4 [M+H]⁺.

### Example 381. Synthesis of trans-3-(6-(1-(2-(1-(4-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 321)

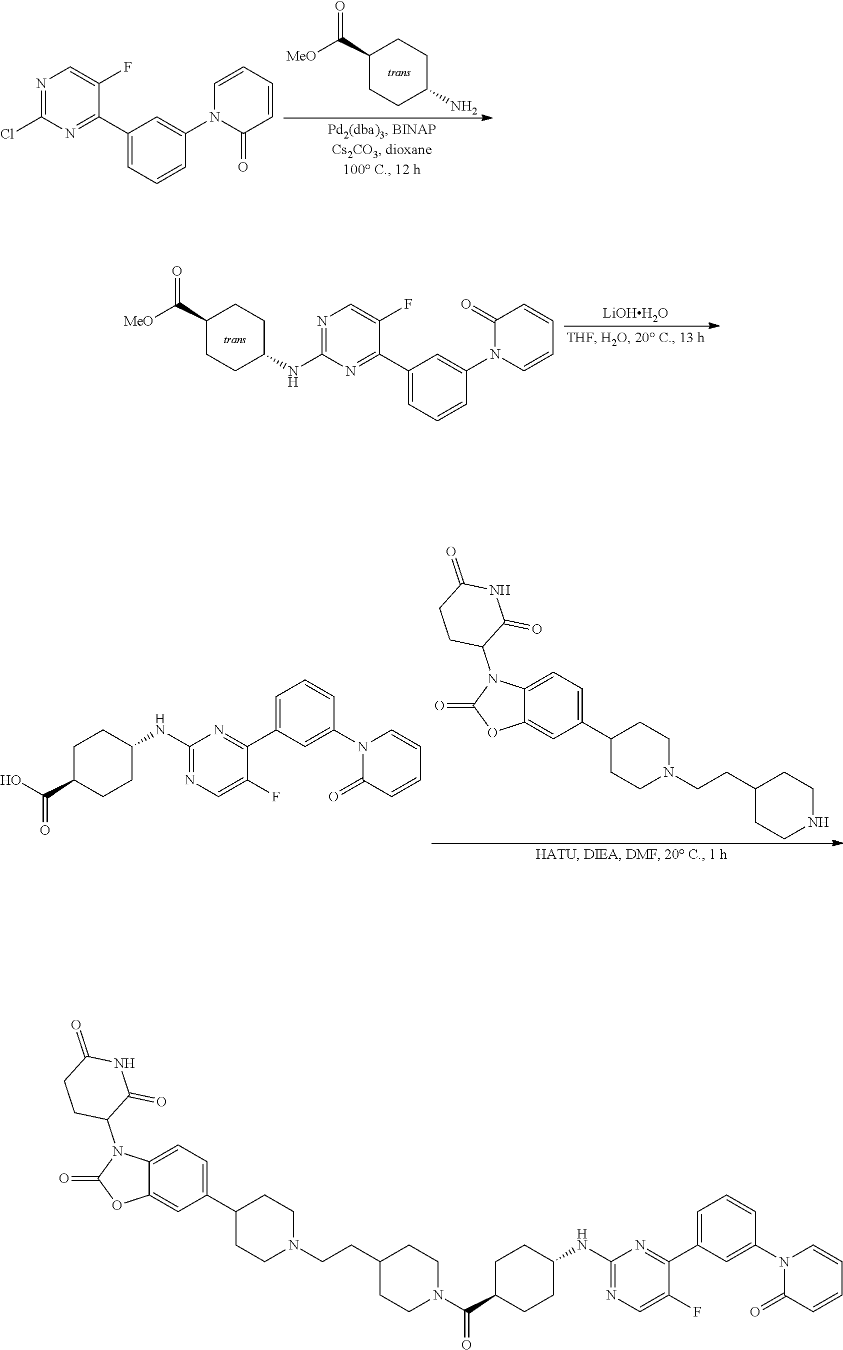

Synthesis of trans-methyl 4-[[5-fluoro-4-[3-(2-oxo-1-pyridyl)phenyl]pyrimidin-2-yl]amino]cyclohexanecarboxylate. A mixture of 1-[3-(2-chloro-5-fluoro-pyrimidin-4-yl)phenyl]pyridin-2-one (100 g, 331 mmol, 1.00 eq), methyl 4-aminocyclohexanecarboxylate (96.3 g, 497 mmol, 1.50 eq, HCl), Cs₂CO₃ (324 g, 994 mmol, 3.00 eq), BINAP (10.3 g, 16.6 mmol, 0.05 eq) and Pd₂(dba)₃ (7.59 g, 8.29 mmol, 0.025 eq) in dioxane (1 L) was degassed and purged with N₂ for 3 times, and then the mixture was stirred at 100 °C for 12 hours under N₂ atmosphere. Two reactions were carried out in parallel. LC-MS showed 1-[3-(2-chloro-5-fluoro-pyrimidin-4-yl)phenyl]pyridin-2-one was consumed completely and desired mass was detected. Two reactions were combined for work-up. The reaction mixture was filtered and concentrated under reduced pressure to give a residue. Then the residue was diluted with H₂O (5 L) and extracted with EtOAc (5 L). The combined organic layers were washed with brine (5 L), dried over Na₂SO₄, filtered, and concentrated under reduced pressure to give a residue. The crude product was triturated with EtOAc (500 mL) at 20 °C for 30 minutes, the filter cake was dried in vacuum to give the title compound (232 g, 549 mmol, 82.8% yield) as a pale yellow solid. LCMS (ES⁺): m/z 423.1 [M+H]⁺.

Synthesis of trans-4-((5-fluoro-4-(3-(2-oxopyridin-1(2H)-yl)phenyl)pyrimidin-2-yl)amino)cyclohexane-1-carboxylic acid. To a solution of trans-methyl 4-[[5-fluoro-4-[3-(2-oxo-1-pyridyl)phenyl]pyrimidin-2-yl]amino]cyclohexanecarboxylate (1.10 g, 2.60 mmol, 1.00 eq) in THF (14 mL) and H₂O (7 mL) was added LiOH·H₂O (328 mg, 7.81 mmol, 3.00 eq) at 20 °C. The mixture was stirred at 20 °C for 13 h. LCMS showed trans-methyl 4-[[5-fluoro-4-[3-(2-oxo-1-pyridyl)phenyl]pyrimidin-2-yl]amino]cyclohexanecarboxylate was consumed completely and the desired mass was detected. HCl (1 M) was added dropwise until the pH was reached 2. The mixture was extracted with EtOAc (10 mL*3). The organic layers were combined, dried over anhydrous Na₂SO₄, filtered and the filtrate was concentrated under reduced pressure to give the title compound (1.13 g, crude) as a green solid. LCMS (ES⁺): m/z 409.2 [M+H]⁺.

Synthesis of trans-3-(6-(1-(2-(1-(4-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 321). To a solution of trans-4-[[5-fluoro-4-[3-(2-oxo-1-pyridyl)phenyl]pyrimidin-2-yl]amino]cyclohexanecarboxylic acid (73.7 mg, 180 μmol, 0.500 eq) in DMF (5 mL) were added HATU (206 mg, 541 μmol, 1.50 eq), DIEA (233 mg, 1.80 mmol, 314 μL, 5.00 eq) and 3-(2-oxo-6-(1-(2-(piperidin-4-yl)ethyl)piperidin-4-yl)benzo[d]oxazol-3 (2H)-yl)piperidine-2,6-dione (200 mg, 361 μmol, 1.00 eq, TFA) at 20 °C. The mixture was stirred at 20 °C for 1 h. LCMS showed 3-(2-oxo-6-(1-(2-(piperidin-4-yl)ethyl)piperidin-4-yl)benzo[d]oxazol-3 (2H)-yl)piperidine-2,6-dione was consumed completely and the desired mass was detected. The mixture was filtered and the filtrate was concentrated under reduced pressure to give a residue. The residue was purified by prep-HPLC (column: 3_Phenomenex Luna C18 75*30 mm*3 um; mobile phase: [H₂O (0.1% TFA)-ACN]; gradient: 5%-35% B over 8.0 min) to give the title compound (Compound 321) (26.4 mg, 31.1 μmol, 8.63% yield, 98.0% purity) as a white solid. LCMS (ES⁺): m/z 831.4 [M+H]⁺

### Example 382. Synthesis of trans-3-((3-fluoro-4-(1-(2-(1-((1r,4r)-4-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)phenyl)amino)piperidine-2,6-dione (Compound 322)

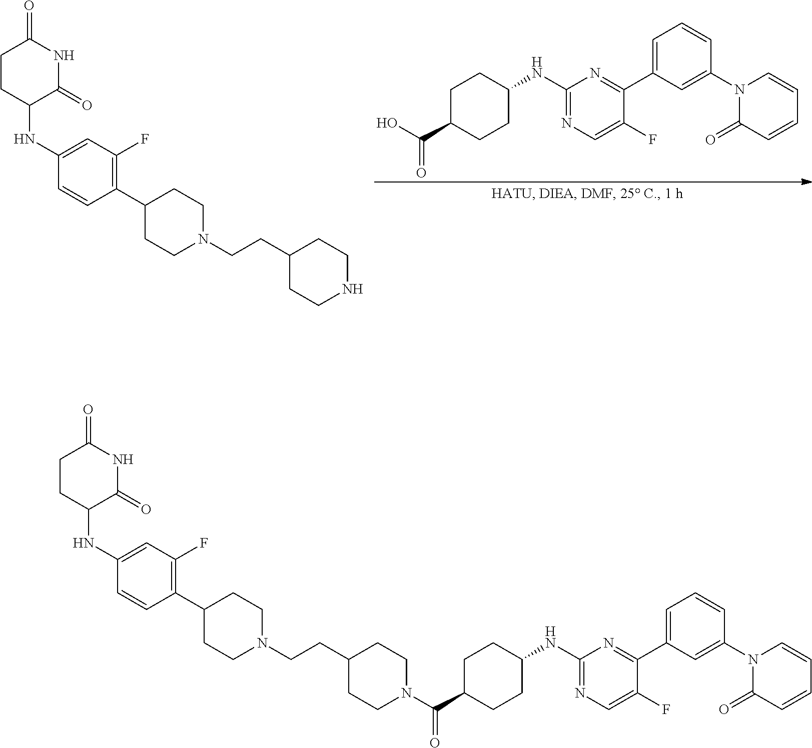 [SMILES: O=C1CCC(Nc2ccc(C3CCN(CCC4CCNCC4)CC3)c(F)c2)C(=O)N1 ; O=c1ccccn1-c1cccc(-c2nc(N[C@H]3CC[C@H](C(=O)O)CC3)ncc2F)c1 ; O=C1CCC(Nc2ccc(C3CCN(CCC4CCN(C(=O)[C@H]5CC[C@H](Nc6ncc(F)c(-c7cccc(-n8ccccc8=O)c7)n6)CC5)CC4)CC3)c(F)c2)C(=O)N1]

Synthesis of trans-3-((3-fluoro-4-(1-(2-(1-((1r,4r)-4-((5-fluoro-4-(3-(2-oxopyridin-1(2H)-yl)phenyl)pyrimidin-2-yl)amino)cyclohexane-1-carbonyl)piperidin-4-yl)ethyl)piperidin-4-yl)phenyl)amino)piperidine-2,6-dione (Compound 322). To a solution of 3-((3-fluoro-4-(1-(2-(piperidin-4-yl)ethyl)piperidin-4-yl)phenyl)amino)piperidine-2,6-dione (85.0 mg, 160 μmol, 1.00 eq, TFA) in DMF (2 mL) were added HATU (67.0 mg, 176 μmol, 1.10 eq), DIEA (62.1 mg, 481 μmol, 83.7 μL, 3.00 eq) and trans-(1r,4r)-4-((5-fluoro-4-(3-(2-oxopyridin-1(2H)-yl)phenyl)pyrimidin-2-yl)amino)cyclohexane-1-carboxylic acid (19.6 mg, 48.1 μmol, 0.300 eq) at 25 °C. The mixture was stirred at 25 °C for 1 h. LCMS showed 3-((3-fluoro-4-(1-(2-(piperidin-4-yl)ethyl)piperidin-4-yl)phenyl)amino)piperidine-2,6-dione was consumed completely and the desired mass was detected. The reaction mixture was concentrated under reduced pressure to remove solvent. The residue was purified by prep-HPLC (column: Phenomenex Luna C18 100*30 mm*3 um; mobile phase: [1H₂O (0.2% FA)-ACN]; gradient: 10%-45% B over 8.0 min) to give the title compound (Compound 322) (17.4 mg, 20.1 μmol, 12.5% yield, 93.0% purity) as a white solid. LCMS (ES⁺): m/z 807.3 [M+H]⁺

### Example 383. Synthesis of trans-3-(6-(1-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)-2-oxobenzo[d]oxazol-3 (2H)-yl)piperidine-2,6-dione (Compound 323)

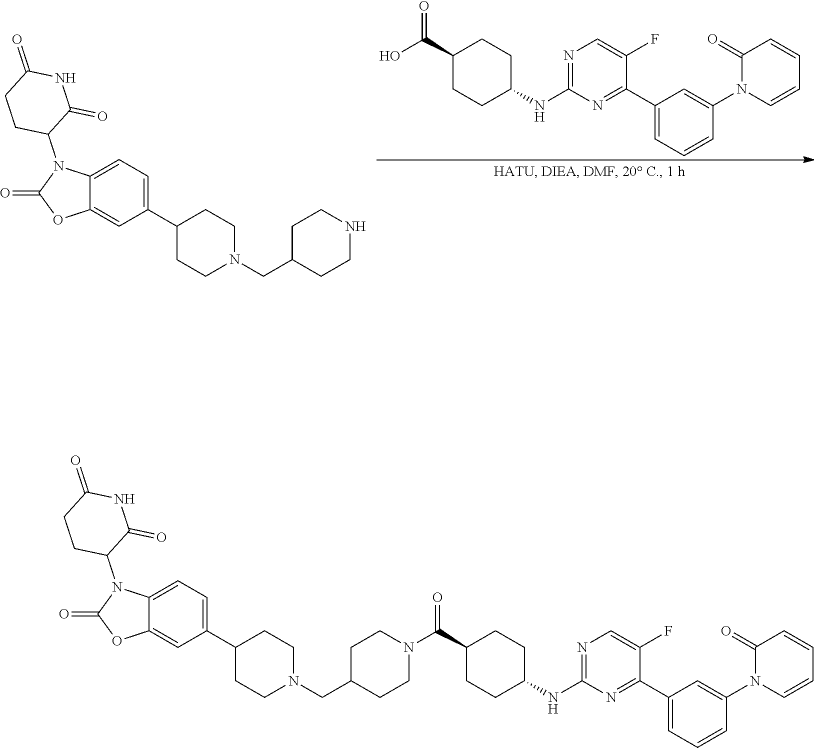 [SMILES: O=C1CCC(n2c(=O)oc3cc(C4CCN(CC5CCNCC5)CC4)ccc32)C(=O)N1 ; O=c1ccccn1-c1cccc(-c2nc(N[C@H]3CC[C@H](C(=O)O)CC3)ncc2F)c1 ; O=C1CCC(n2c(=O)oc3cc(C4CCN(CC5CCN(C(=O)[C@H]6CC[C@H](Nc7ncc(F)c(-c8cccc(-n9ccccc9=O)c8)n7)CC6)CC5)CC4)ccc32)C(=O)N1]

Synthesis of trans-3-(6-(1-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)-2-oxobenzo[d]oxazol-3 (2H)-yl)piperidine-2,6-dione (Compound 323). To a solution of trans-4-[[5-fluoro-4-[3-(2-oxo-1-pyridyl)phenyl]pyrimidin-2-yl]amino]cyclohexanecarboxylic acid (57.3 mg, 140 μmol, 0.500 eq) in DMF (5 mL) were added HATU (160 mg, 421 μmol, 1.50 eq), DIEA (181 mg, 1.40 mmol, 245 μL, 5.00 eq) and 3-(2-oxo-6-(1-(piperidin-4-ylmethyl)piperidin-4-yl)benzo[d]oxazol-3 (2H)-yl)piperidine-2,6-dione (130 mg, 281 μmol, 1.00 eq, HCl) at 20 °C. The mixture was stirred at 20 °C for 1 h. LCMS showed 3-(2-oxo-6-(1-(piperidin-4-ylmethyl)piperidin-4-yl)benzo[d]oxazol-3 (2H)-yl)piperidine-2,6-dione was consumed completely and the desired mass was detected. The mixture was filtered and the filtrate was concentrated under reduced pressure to give a residue. The residue was purified by prep-HPLC (column: 3_Phenomenex Luna C18 75*30 mm*3 um; mobile phase: [H₂O (0.1% TFA)-ACN]; gradient: 20%-50% B over 8.0 min) to give the title compound (Compound 323) (7.20 mg, 8.78 μmol, 3.13% yield, 99.6% purity) as a white solid. LCMS (ES⁺): m/z 817.3[M+H]⁺

### Example 384. Synthesis of trans-3-((3-fluoro-4-(1-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)phenyl)amino)piperidine-2,6-dione (Compound 318)

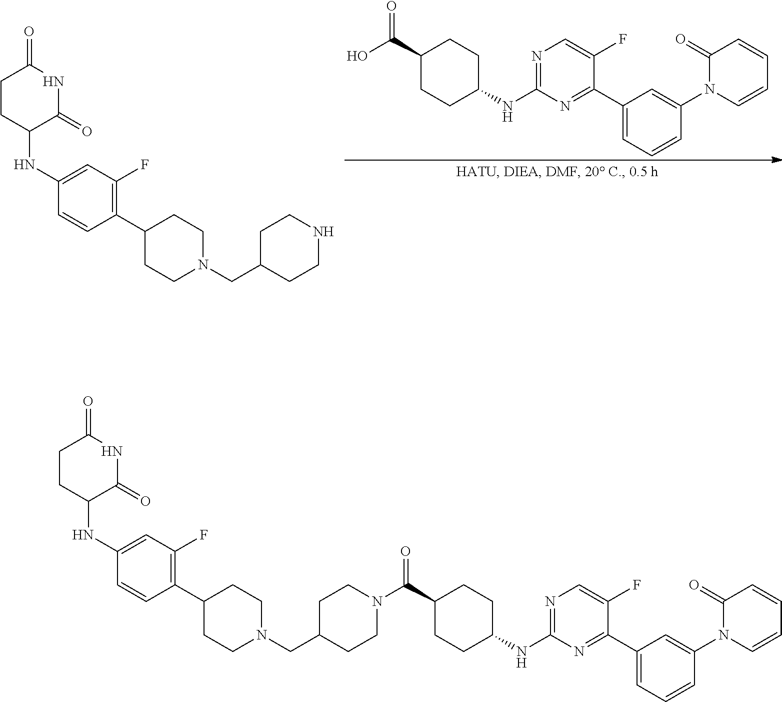 [SMILES: O=C1CCC(Nc2ccc(C3CCN(CC4CCNCC4)CC3)c(F)c2)C(=O)N1 ; O=c1ccccn1-c1cccc(-c2nc(N[C@H]3CC[C@H](C(=O)O)CC3)ncc2F)c1 ; O=C1CCC(Nc2ccc(C3CCN(CC4CCN(C(=O)[C@H]5CC[C@H](Nc6ncc(F)c(-c7cccc(-n8ccccc8=O)c7)n6)CC5)CC4)CC3)c(F)c2)C(=O)N1]

Synthesis of trans-3-((3-fluoro-4-(1-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)phenyl)amino)piperidine-2,6-dione (Compound 318). To a solution of trans-4-((5-fluoro-4-(3-(2-oxopyridin-1(2H)-yl)phenyl)pyrimidin-2-yl)amino)cyclohexane-1-carboxylic acid (142 mg, 348 μmol, 1.00 eq) in DMF (3 mL) were added HATU (199 mg, 523 μmol, 1.50 eq), DIEA (225 mg, 1.74 mmol, 304 μL, 5.00 eq) and 3-((3-fluoro-4-(1-(piperidin-4-ylmethyl)piperidin-4-yl)phenyl)amino)piperidine-2,6-dione (180 mg, 348 μmol, 1.00 eq, TFA) at 20 °C, then the mixture was stirred at 20 °C for 0.5 h. LCMS showed 3-((3-fluoro-4-(1-(piperidin-4-ylmethyl)piperidin-4-yl)phenyl)amino)piperidine-2,6-dione was consumed and the desired target MS was detected. The mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 15%-40% B over 8.0 min) to give the title compound (Compound 318) (33.5 mg, 41.1 μmol, 11.2% yield, 97.2% purity) as a white solid. LCMS (ES⁺): m/z 793.4 [M+H]⁺

### Example 385. Synthesis of trans-(R)-3-((3-fluoro-4-(1-((1-((1r,4r)-4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)phenyl)amino)piperidine-2,6-dione (Compound 455) and trans-(S)-3-((3-fluoro-4-(1-((1-((1r,4r)-4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)phenyl)amino)piperidine-2,6-dione (Compound 456) (The Two Isomers were Assigned Arbitrarily)

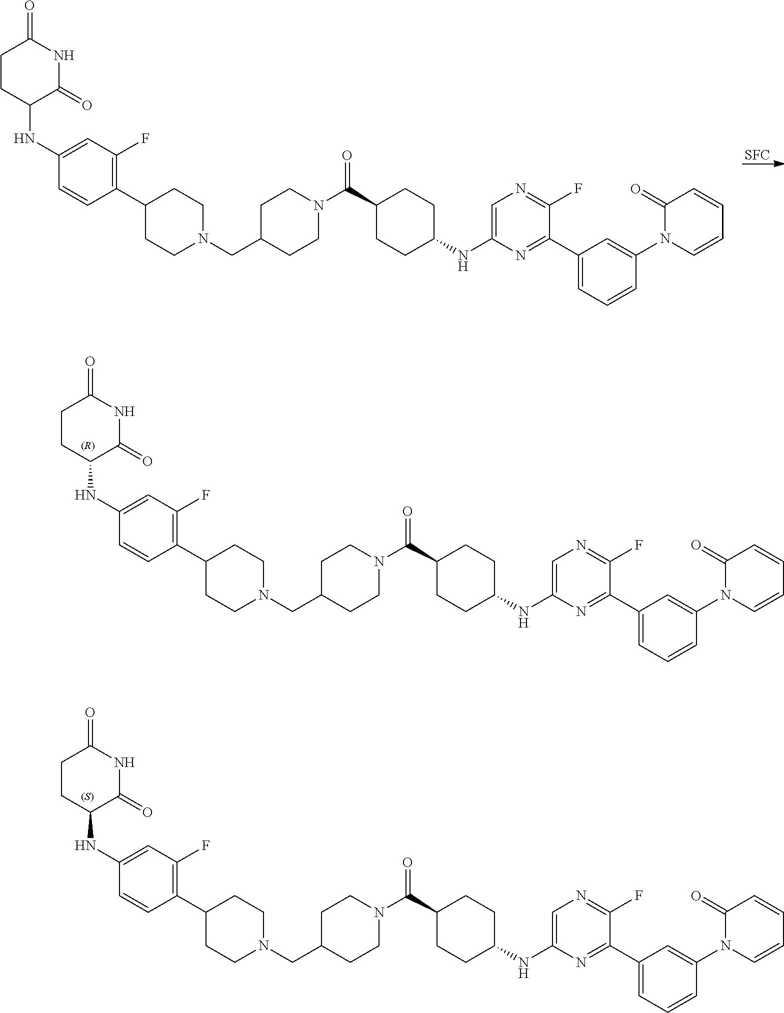

Synthesis of trans-(R)-3-((3-fluoro-4-(1-((1-((1r,4r)-4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)phenyl)amino)piperidine-2,6-dione (Compound 455) and trans-(S)-3-((3-fluoro-4-(1-((1-((1r,4r)-4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)phenyl)amino)piperidine-2,6-dione (Compound 456) (the two isomers were assigned arbitrarily). Trans-3-((3-fluoro-4-(1-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)phenyl)amino)piperidine-2,6-dione (Compound 318) (4.30 g, 5.42 mmol, 1.00 eq) was separated by chiral SFC (column: DAICEL CHIRALCEL OD (250 mm*30 mm, 10 um); mobile phase: [CO₂—IPA (0.1% IPAm)]; B %: 56%, isocratic elution mode) to give the title compound (Compound 455) (837 mg, 983 μmol, 18.1% yield, 93.1% purity, 100% cc) (retention time: 2.522, peak 1) as a white solid. LCMS (ES+): m/z 793.4 [M+H]+ and title compound (Compound 456) (563 mg, 687 μmol, 12.7% yield, 96.7% purity, 97.62% ee) (retention time: 4.726, peak 2) as a white solid. LCMS (ES+): m/z 793.4 [M+H]⁺

### Example 386. Synthesis of trans-N-(1′-(4-(((R)-2,6-dioxopiperidin-3-yl)amino)-2-fluorophenyl)-[1,4′-bipiperidin]-4-yl)-4-((5-fluoro-4-(3-(2-oxopyridin-1(2H)-yl)phenyl)pyrimidin-2-yl)amino)cyclohexane-1-carboxamide (Compound 440)

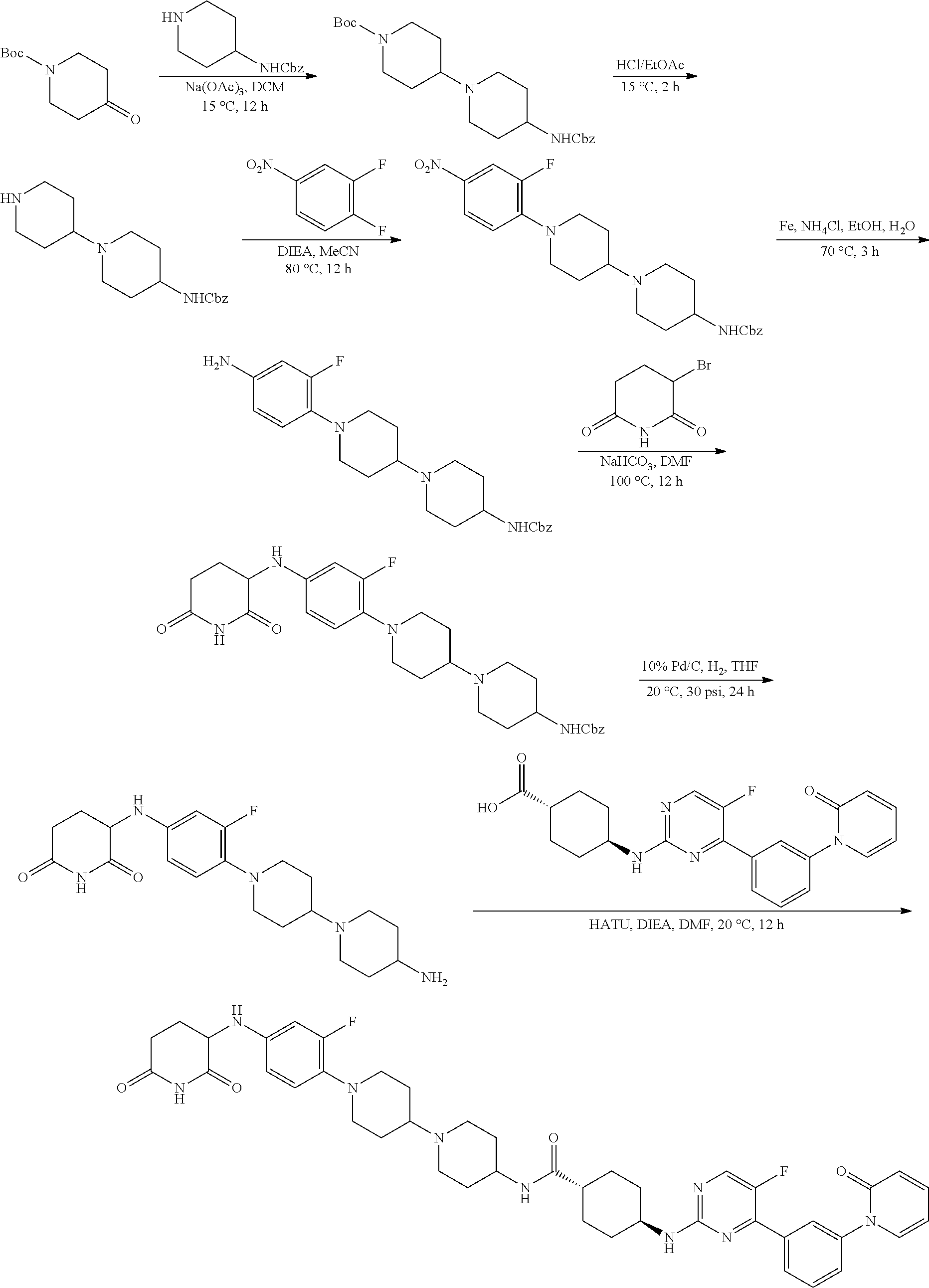

Synthesis of tert-butyl 4-(((benzyloxy)carbonyl)amino)-[1,4′-bipiperidine]-1′-carboxylate. To a solution of tert-butyl 4-oxopiperidine-1-carboxylate (10.0 g, 50.2 mmol, 1.00 eq) and benzyl N-(4-piperidyl) carbamate (11.8 g, 50.2 mmol, 1.00 eq) in DCM (100 mL) was added NaBH(OAc)₃ (31.9 g, 151 mmol, 3.00 eq) at 15 °C. The mixture was stirred at 15 °C for 2 h. LCMS showed that tert-butyl 4-oxopiperidine-1-carboxylate was consumed completely and one main peak with desired mass was detected. The mixture was diluted with H₂O (200 mL) and extracted with DCM (50 mL*3). The organic layers were washed with brine (150 mL) dried over Na₂SO₄, filtered and concentrated under reduced pressure. The residue was purified by MPLC (SiO₂, Petroleum ether/THF=1/0 to 0/1) to give the title compound (17.3 g, 41.4 mmol, 82.6% yield) as a colourless oil. LCMS (ES⁺): m/z 418.2 [M+H]⁺.

Synthesis of benzyl [1,4′-bipiperidin]-4-ylcarbamate. To a solution of 4-(((benzyloxy)carbonyl)amino)-[1,4′-bipiperidine]-1′-carboxylate (17.3 g, 41.4 mmol, 1.00 eq) and in HCl/EtOAc (4 M, 50 mL) was stirred at 15 °C for 2 h. LCMS showed that 4-(((benzyloxy)carbonyl)amino)-[1,4′-bipiperidine]-1′-carboxylate was consumed completely and one main peak with desired mass was detected. The mixture was concentrated under reduced pressure to give the title compound (15.0 g, crude, HCl) as a white solid. LCMS (ES⁺): m/z 318.2 [M+H]⁺.

Synthesis of benzyl (1′-(2-fluoro-4-nitrophenyl)-[1,4′-bipiperidin]-4-yl)carbamate. To a solution of benzyl [1,4′-bipiperidin]-4-ylcarbamate (15.0 g, 42.4 mmol, 1.00 eq, HCl) and 1,2-difluoro-4-nitro-benzene (8.09 g, 50.9 mmol, 5.63 mL, 1.20 eq) in MeCN (150 mL) was added DIPEA (16.4 g, 127 mmol, 22.2 mL, 3.00 eq) at 15 °C. The mixture was stirred at 80 °C for 12 h. LCMS showed that benzyl [1,4′-bipiperidin]-4-ylcarbamate was consumed completely and one main peak with desired mass was detected. The mixture was filtered and the filter cake was dried over in vacuum to give title compound (15.1 g, crude) as yellow solid. LCMS (ES⁺): m/z 457.2 [M+H]⁺.

Synthesis of benzyl (1′-(4-amino-2-fluorophenyl)-[1,4′-bipiperidin]-4-yl)carbamate. To a mixture of benzyl (1′-(2-fluoro-4-nitrophenyl)-[1,4′-bipiperidin]-4-yl)carbamate (15.1 g, 33.1 mmol, 1.00 eq) and Fe (9.24 g, 165 mmol, 5.00 eq) in EtOH (40 mL) and THE (40 mL) was added dropwise NH₄Cl (4.42 g, 82.7 mmol, 2.50 eq) in H₂O (40 mL) at 20 °C. The mixture was stirred at 70 °C for 3 h. LCMS showed that benzyl (1′-(2-fluoro-4-nitrophenyl)-[1,4′-bipiperidin]-4-yl)carbamate was consumed completely and one main peak with desired mass was detected. The mixture was filtered and the filtrate was concentrated under reduced pressure to give title compound (15.0 g, crude) as yellow solid. LCMS (ES⁺): m/z 427.2 [M+H]⁺.

Synthesis of rac-benzyl (R)-(1′-(4-((2,6-dioxopiperidin-3-yl)amino)-2-fluorophenyl)-[1,4′-bipiperidin]-4-yl)carbamate. To a solution of benzyl (1′-(4-amino-2-fluorophenyl)-[1,4′-bipiperidin]-4-yl)carbamate (7.00 g, 16.4 mmol, 1.00 eq) and rac-3-bromopiperidine-2,6-dione (12.6 g, 65.7 mmol, 4.00 eq) in DMF (50 mL) was added NaHCO₃ (2.76 g, 32.8 mmol, 1.28 mL, 2.00 eq) at 20 °C. The mixture was stirred at 100 °C for 12 h. LCMS showed that benzyl (1′-(4-amino-2-fluorophenyl)-[1,4′-bipiperidin]-4-yl)carbamate was consumed completely and one main peak with desired MS was detected. The reaction mixture was concentrated was diluted with H₂O (150 mL) and extracted with THF/EtOAc=2/1 (500 mL*3). The combined organic layers were dried over anhydrous Na₂SO₄, filtered and concentrated under reduced pressure. The crude product was purified by reversed-phase HPLC (FA condition, 10-40% 30 min; 40% 15 min) to give title compound (4.00 g, 7.44 mmol, 45.3% yield) as a gray solid. LCMS (ES⁺): m/z 538.2 [M+H]⁺.

Synthesis of rac-3-((4-(4-amino-[1,4′-bipiperidin]-1′-yl)-3-fluorophenyl)amino)piperidine-2,6-dione. To a mixture of 10% Pd/C (1.00 g, 50% purity) in THE (50 mL) was added rac-benzyl rac-(1′-(4-((2,6-dioxopiperidin-3-yl)amino)-2-fluorophenyl)-[1,4′-bipiperidin]-4-yl)carbamate (3.00 g, 5.58 mmol, 1.00 eq) at 20 °C. The suspension was degassed and purged with H₂ for 3 times. The mixture was stirred under H₂ (30 Psi) at 20 °C for 24 h. LC-MS showed that rac-benzyl (R)-(1′-(4-((2,6-dioxopiperidin-3-yl)amino)-2-fluorophenyl)-[1,4′-bipiperidin]-4-yl)carbamate was remained and one main peak with desired MS was detected. The reaction mixture was filtered and the filtrate was concentrated under reduced pressure. The residue was purified by prep-HPLC (column: Phenomenex luna C18 (250*70 mm, 15 um); mobile phase: [H₂O (0.2% FA)-ACN]; gradient: 1%-20% B over 20.0 min) to give title compound (450 mg, 1.12 mmol, 20.0% yield, N/A purity) as an off-white solid. LCMS (ES⁺): m/z 404.2 [M+H]⁺.

Synthesis of trans-N-(1′-(4-(((R)-2,6-dioxopiperidin-3-yl)amino)-2-fluorophenyl)-[1,4′-bipiperidin]-4-yl)-4-((5-fluoro-4-(3-(2-oxopyridin-1(2H)-yl)phenyl)pyrimidin-2-yl)amino)cyclohexane-1-carboxamide (Compound 440). To a solution of rac-3-((4-(4-amino-[1,4′-bipiperidin]-1′-yl)-3-fluorophenyl)amino)piperidine-2,6-dione (131 mg, 320 μmol, 0.300 eq) in DMF (4 mL) was added HATU (800 mg, 2.10 mmol, 1.97 eq), DIEA (413 mg, 3.20 mmol, 557 μL, 3.00 eq) and rac-(1r,4r)-4-((5-fluoro-4-(3-(2-oxopyridin-1(2H)-yl)phenyl)pyrimidin-2-yl)amino)cyclohexane-1-carboxylic acid (430 mg, 1.07 mmol, 1.00 eq) at 20 °C. The mixture was stirred at 20 °C for 12 h. LC-MS showed that rac-(R)-3-((4-(4-amino-[1,4′-bipiperidin]-1′-yl)-3-fluorophenyl)amino)piperidine-2,6-dione was consumed completely and one main peak with desired MS was detected. The reaction mixture was purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min) to give title compound (Compound 440) (32.0 mg, 37.2 μmol, 11.6% yield, 92.2% purity) as a pale yellow solid. LCMS (ES⁺): m/z 794.4 [M+H]⁺.

### Example 387. Synthesis of trans-3-((3-fluoro-4-(4-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-1-yl)phenyl)amino)piperidine-2,6-dione (Compound 400)

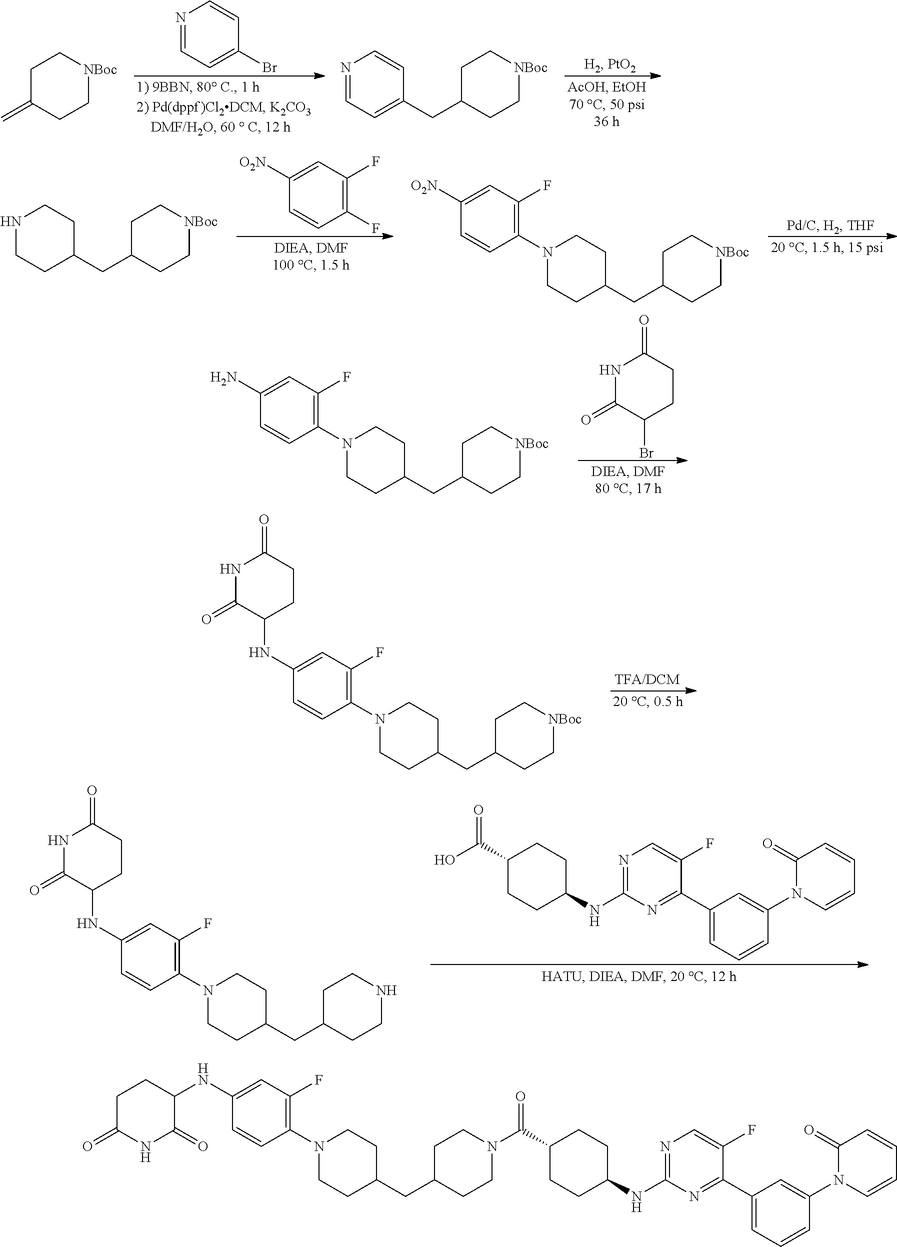

Synthesis of tert-butyl 4-(pyridin-4-ylmethyl)piperidine-1-carboxylate. A solution of tert-butyl 4-methylenepiperidine-1-carboxylate (3.00 g, 15.2 mmol, 1.00 eq) in 9-BBN (0.5 M, 30.4 mL, 1.00 eq) was stirred at 80 °C for 1 h under N₂, after cooling to 20 °C, 4-bromopyridine (2.16 g, 13.7 mmol, 0.900 eq), K₂CO₃ (2.52 g, 18.3 mmol, 1.20 eq), Pd(dppf)Cl₂·CH₂Cl₂ (373 mg, 456 μmol, 0.0300 eq) in DMF (25 mL) and H₂O (2.5 mL) were added to the mixture, the mixture was stirred at 60 °C for 12 h under N₂. LCMS showed tert-butyl 4-methylenepiperidine-1-carboxylate was consumed and the desired target MS was detected. H₂O (25 mL) was added to the mixture and the mixture was extracted with EtOAc (25 mL*3), the combined organic layers were washed with brine (25 mL*3), dried over Na₂SO₄, filtered and the filtrate was concentrated under reduced pressure to give a residue. The residue was purified by MPLC (SiO₂, PE/THF=1/0 to 0/1) to give the title compound (2.40 g, 8.68 mmol, 57.1% yield) as a yellow oil. LCMS (ES⁺): m/z 277.2 [M+H]⁺

Synthesis of tert-butyl 4-(piperidin-4-ylmethyl)piperidine-1-carboxylate. To PtO₂ (123 mg, 543 μmol, 0.15 eq) in EtOH (20 mL) and AcOH (217 mg, 3.62 mmol, 207 μL, 1.00 eq) was added tert-butyl 4-(pyridin-4-ylmethyl)piperidine-1-carboxylate (1.00 g, 3.62 mmol, 1.00 eq) at 20 °C under N₂, the suspension was degassed and purged with H₂ for three times. The mixture was stirred at 70 °C for 12 h under H₂ (50 psi). LCMS showed tert-butyl 4-(pyridin-4-ylmethyl)piperidine-1-carboxylate remained and the desired target MS was detected. Then AcOH (217 mg, 3.62 mmol, 207 μL, 1.00 eq) and PtO₂ (123 mg, 543 μmol, 0.150 eq) was added to the mixture at 20 °C under N₂, the suspension was degassed and purged with H₂ for three times. The mixture was stirred at 70 °C for 24 h under H₂ (50 psi). LCMS showed tert-butyl 4-(pyridin-4-ylmethyl)piperidine-1-carboxylate was consumed completely and the desired target MS was detected. The mixture were filtered and the filtrate was concentrated under reduced pressure to give the title compound (1.60 g, crude) as a brown oil. LCMS (ES⁺): m/z 283.2 [M+H]⁺

Synthesis of tert-butyl 4-((1-(2-fluoro-4-nitrophenyl)piperidin-4-yl)methyl)piperidine-1-carboxylate. To a solution of tert-butyl 4-(piperidin-4-ylmethyl)piperidine-1-carboxylate (1.00 g, 3.54 mmol, 1.00 eq) and 1,2-difluoro-4-nitrobenzene (563 mg, 3.54 mmol, 392 μL, 1.00 eq) in DMF (15 mL) was added DIEA (961 mg, 7.44 mmol, 1.30 mL, 3.00 eq) at 20 °C. The mixture was stirred at 100 °C for 1.5 h. LCMS showed tert-butyl 4-(piperidin-4-ylmethyl)piperidine-1-carboxylate was consumed and the desired target MS was detected. H₂O (15 mL) was added to the mixture, then the mixture was filtered, the filter cake was dried under reduced pressure to give the title compound (870 mg, crude) as a brown solid. LCMS (ES⁺): m/z 366.2 [M+H−56]⁺

Synthesis of tert-butyl 4-((1-(4-amino-2-fluorophenyl)piperidin-4-yl)methyl)piperidine-1-carboxylate. To a mixture of tert-butyl 4-((1-(2-fluoro-4-nitrophenyl)piperidin-4-yl)methyl)piperidine-1-carboxylate (870 mg, 2.06 mmol, 1.00 eq) in THF (20 mL) was added 10% Pd/C (500 mg, 50% purity) at 20 °C under N₂, the suspension was degassed and purged with H₂ for three times. The mixture was stirred at 20 °C for 1.5 h under H₂ (15 psi). LCMS showed tert-butyl 4-((1-(2-fluoro-4-nitrophenyl)piperidin-4-yl)methyl)piperidine-1-carboxylate was consumed and the desired target MS was detected. The mixture was filtered, the filtrate was concentrated under reduced pressure to give the title compound (800 mg, crude) as a brown oil. LCMS (ES⁺): m/z 392.3 [M+H]⁺

Synthesis of tert-butyl 4-((1-(4-((2,6-dioxopiperidin-3-yl)amino)-2-fluorophenyl)piperidin-4-yl)methyl)piperidine-1-carboxylate. To a solution of tert-butyl 4-((1-(4-amino-2-fluorophenyl)piperidin-4-yl)methyl)piperidine-1-carboxylate (800 mg, 2.04 mmol, 1.00 eq) and 3-bromopiperidine-2,6-dione (1.18 g, 6.13 mmol, 3.00 eq) in DMF (15 mL) was added DIEA (792 mg, 6.13 mmol, 1.07 mL, 3.00 eq) at 20 °C, the mixture was stirred at 80 °C for 17 h. LCMS showed tert-butyl 4-((1-(4-amino-2-fluorophenyl)piperidin-4-yl)methyl)piperidine-1-carboxylate was consumed completely and the desired target MS was detected. H₂O (20 mL) was added to the mixture and the mixture was extracted with EtOAc (20 mL*3), the combined organic layers were washed with brine (20 mL*3), dried over Na₂SO₄, filtered and the filtrate was concentrated under reduced pressure to give a residue. The residue was purified by prep-TLC (SiO₂, PE/EtOAc=0/1) to give the title compound (1.00 g, 1.99 mmol, 97.4% yield) as a brown oil. LCMS (ES⁺): m/z 503.3 [M+H]⁺

Synthesis of 3-((3-fluoro-4-(4-(piperidin-4-ylmethyl)piperidin-1-yl)phenyl)amino)piperidine-2,6-dione. A solution of tert-butyl 4-((1-(4-((2,6-dioxopiperidin-3-yl)amino)-2-fluorophenyl)piperidin-4-yl)methyl)piperidine-1-carboxylate (1.00 g, 1.99 mmol, 1.00 eq) in DCM (10 mL) and TFA (3 mL) was stirred at 20 °C for 0.5 h. LCMS showed tert-butyl 4-((1-(4-((2,6-dioxopiperidin-3-yl)amino)-2-fluorophenyl)piperidin-4-yl)methyl)piperidine-1-carboxylate was consumed and the desired target MS was detected. The mixture was concentrated under reduced pressure to give title compound (1.00 g, crude, TFA) as a brown oil. LCMS (ES⁺): m/z 403.3 [M+H]⁺

Synthesis of trans-3-((3-fluoro-4-(4-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-1-yl)phenyl)amino)piperidine-2,6-dione (Compound 400). To a solution of 3-((3-fluoro-4-(4-(piperidin-4-ylmethyl)piperidin-1-yl)phenyl)amino)piperidine-2,6-dione (2.90 g, 5.61 mmol, 1.00 eq, TFA) and trans-(1r,4r)-4-((5-fluoro-4-(3-(2-oxopyridin-1(2H)-yl)phenyl)pyrimidin-2-yl)amino)cyclohexane-1-carboxylic acid (229 mg, 561 μmol, 0.100 eq) in DMF (20 mL) were added DIEA (2.18 g, 16.8 mmol, 2.93 mL, 3.00 eq) and HATU (4.27 g, 11.2 mmol, 2.00 eq) at 20 °C. The mixture was stirred at 20 °C for 12 h. LC-MS showed that 3-((3-fluoro-4-(4-(piperidin-4-ylmethyl)piperidin-1-yl)phenyl)amino)piperidine-2,6-dione was consumed completely and desired mass was detected. The residue was diluted with H₂O (20 mL) and extracted with EtOAc (20 mL*3). The combined organic layers were washed with brine (30 mL), dried over NA₂SO₄, filtered and concentrated under reduced pressure to give a residue. The residue was purified by prep-HPLC (column: Phenomenex luna C18 (250*70 mm, 15 um); mobile phase: [H₂O (0.2% FA)-ACN]; gradient: 30%-60% B over 20.0 min) to give the title compound (Compound 400) (56.5 mg, 70.6 μmol, 1.26% yield, 99.1% purity) as a white solid. LCMS (ES⁺): m/z 793.4 [M+H]⁺

### Example 388. Synthesis of trans-3-((3-fluoro-4-(4-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperazin-1-yl)phenyl)amino)piperidine-2,6-dione (Compound 374)

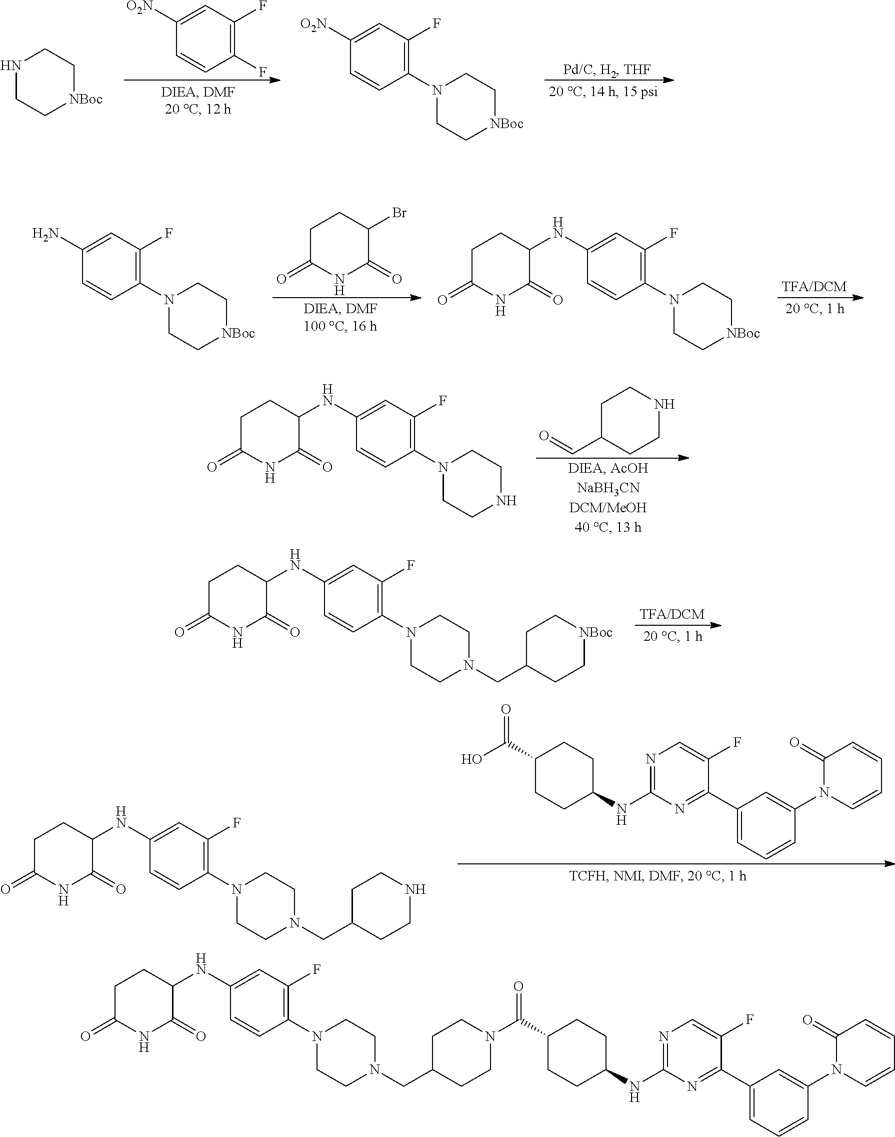

Synthesis of tert-butyl 4-(2-fluoro-4-nitrophenyl)piperazine-1-carboxylate. To a solution of tert-butyl piperazine-1-carboxylate (10.0 g, 53.7 mmol, 1.00 eq) in DMF (150 mL) were added DIEA (20.8 g, 161 mmol, 28.1 mL, 3.00 eq) and 1,2-difluoro-4-nitrobenzene (8.54 g, 53.7 mmol, 5.94 mL, 1.00 eq) at 20 °C. The mixture was stirred at 20 °C for 12 h. LCMS showed that the desired mass was detected. To the mixture was added H₂O (100 mL), then the mixture was filtered and the filter cake was dried under reduced pressure to give the title compound (11.7 g, crude) as a yellow solid. LCMS (ES⁺): m/z 270.1 [M+H−56]⁺.

Synthesis of tert-butyl 4-(4-amino-2-fluorophenyl)piperazine-1-carboxylate. To a mixture of tert-butyl 4-(2-fluoro-4-nitrophenyl)piperazine-1-carboxylate (11.7 g, 36.0 mmol, 1.00 eq) in THE (350 mL) was added 10% Pd/C (8.00 g, 50% purity) at 20 °C under N₂. The suspension was degassed and purged with H₂ for 3 times. The mixture was stirred under H₂ (15 psi) at 20 °C for 14 h. LCMS showed tert-butyl 4-(2-fluoro-4-nitrophenyl)piperazine-1-carboxylate was consumed completely and the desired mass was detected. The mixture was filtered and the filtrate was concentrated under reduced pressure to give the title compound (10.5 g, crude) as a yellow oil. LCMS (ES⁺): m/z 296.1 [M+H]⁺.

Synthesis of tert-butyl 4-(4-((2,6-dioxopiperidin-3-yl)amino)-2-fluorophenyl)piperazine-1-carboxylate. To a solution of tert-butyl 4-(4-amino-2-fluorophenyl)piperazine-1-carboxylate (10.5 g, 35.5 mmol, 1.00 eq) in DMF (200 mL) were added DIEA (13.8 g, 107 mmol, 18.6 mL, 3.00 eq) and 3-bromopiperidine-2,6-dione (13.6 g, 71.0 mmol, 2.00 eq) at 20 °C. The mixture was stirred at 100 °C for 16 h. LCMS showed tert-butyl 4-(4-amino-2-fluorophenyl)piperazine-1-carboxylate was consumed completely and the desired mass was detected. To the mixture was added H₂O (200 mL), then the mixture was filtered and the filter cake was dried under reduced pressure to give the title compound (6.03 g, crude) as a brown solid. LCMS (ES⁺): m/z 407.3 [M+H]⁺.

Synthesis of 3-((3-fluoro-4-(piperazin-1-yl)phenyl)amino)piperidine-2,6-dione. A solution of tert-butyl 4-(4-((2,6-dioxopiperidin-3-yl)amino)-2-fluorophenyl)piperazine-1-carboxylate (4.00 g, 9.84 mmol, 1.00 eq) in DCM (30 mL) and TFA (10 mL) was stirred at 20 °C for 1 h. LCMS shows tert-butyl 4-(4-((2,6-dioxopiperidin-3-yl)amino)-2-fluorophenyl)piperazine-1-carboxylate was consumed and the desired mass was detected. The reaction mixture was concentrated under reduced pressure to give the title compound (4.14 g, crude, TFA) as a brown oil. LCMS (ES⁺): m/z 307.1 [M+H]⁺.

Synthesis of tert-butyl 4-((4-(4-((2,6-dioxopiperidin-3-yl)amino)-2-fluorophenyl)piperazin-1-yl)methyl)piperidine-1-carboxylate. To a solution of 3-((3-fluoro-4-(piperazin-1-yl)phenyl)amino)piperidine-2,6-dione (4.14 g, 9.85 mmol, 1.00 eq, TFA) in MeOH (20 mL) and DCM (60 mL) was added DIEA to adjust pH to 8 at 20 °C. The mixture was stirred at 20 °C for 0.5 h. AcOH (1.77 g, 29.6 mmol, 1.69 mL, 3.00 eq) and tert-butyl 4-formylpiperidine-1-carboxylate (2.52 g, 11.8 mmol, 1.20 eq) were added to the mixture at 20 °C, pH was 5 now. The mixture was stirred at 20 °C for 0.5 h. Then NaBH₃CN (1.86 g, 29.6 mmol, 3.00 eq) was added to the mixture at 20 °C. The mixture was stirred at 40 °C for 12 h. LCMS showed 3-((3-fluoro-4-(piperazin-1-yl)phenyl)amino)piperidine-2,6-dione was consumed completely and desired mass was detected. The reaction mixture was concentrated under reduced pressure to give a residue. The residue was diluted with H₂O (60 mL) and then extracted with EtOAc (30 mL*3). The combined organic layers were dried over Na₂SO₄, filtered and the filtrate was concentrated under reduced pressure to give a residue. The residue was purified by MPLC (SiO₂, DCM/EtOAc=1/0 to 1/3) to give the title compound (2.76 g, 5.48 mmol, 55.6% yield) as a yellow oil. LCMS (ES⁺): m/z 504.2 [M+H]⁺.

Synthesis of 3-((3-fluoro-4-(4-(piperidin-4-ylmethyl)piperazin-1-yl)phenyl)amino)piperidine-2,6-dione. A solution of tert-butyl 4-((4-(4-((2,6-dioxopiperidin-3-yl)amino)-2-fluorophenyl)piperazin-1-yl)methyl)piperidine-1-carboxylate (1.00 g, 1.99 mmol, 1.00 eq) in DCM (10 mL) and TFA (3.3 mL) was stirred at 20 °C for 1 h. LCMS shows tert-butyl 4-((4-(4-((2,6-dioxopiperidin-3-yl)amino)-2-fluorophenyl)piperazin-1-yl)methyl)piperidine-1-carboxylate was consumed and the desired mass was detected. The reaction mixture was concentrated under reduced pressure to give the title compound (1.03 g, crude, TFA) as a yellow oil. LCMS (ES⁺): m/z 404.3 [M+H]⁺.

Synthesis of trans-3-((3-fluoro-4-(4-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperazin-1-yl)phenyl)amino)piperidine-2,6-dione (Compound 374). To a solution of trans-4-((5-fluoro-4-(3-(2-oxopyridin-1(2H)-yl)phenyl)pyrimidin-2-yl)amino)cyclohexane-1-carboxylic acid (677 mg, 1.66 mmol, 0.500 eq) in DMF (10 mL) were added TCFH (670 mg, 2.39 mmol, 1.20 eq) and NMI (490 mg, 5.97 mmol, 793 μL, 5.00 eq) at 20 °C, then 3-((3-fluoro-4-(4-(piperidin-4-ylmethyl)piperazin-1-yl)phenyl)amino)piperidine-2,6-dione (1.03 g, 1.99 mmol, 1.00 eq, TFA) was added to the mixture, the mixture was stirred at 20 °C for 1 h. LCMS showed 3-((3-fluoro-4-(4-(piperidin-4-ylmethyl)piperazin-1-yl)phenyl)amino)piperidine-2,6-dione was consumed completely and the desired mass was detected. The reaction mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 10%-45% B over 8.0 min) to give the title compound (Compound 374) (92.3 mg, 109 μmol, 5.50% yield, 94.1% purity) as a brown solid. LCMS (ES⁺): m/z 794.4 [M+H]⁺

### Example 389. Synthesis of trans-N-(1-((1-(4-((2,6-dioxopiperidin-3-yl)amino)-2-fluorophenyl)piperidin-4-yl)methyl)piperidin-4-yl)-4-((5-fluoro-4-(3-(2-oxopyridin-1(2H)-yl)phenyl)pyrimidin-2-yl)amino)cyclohexane-1-carboxamide (Compound 375)

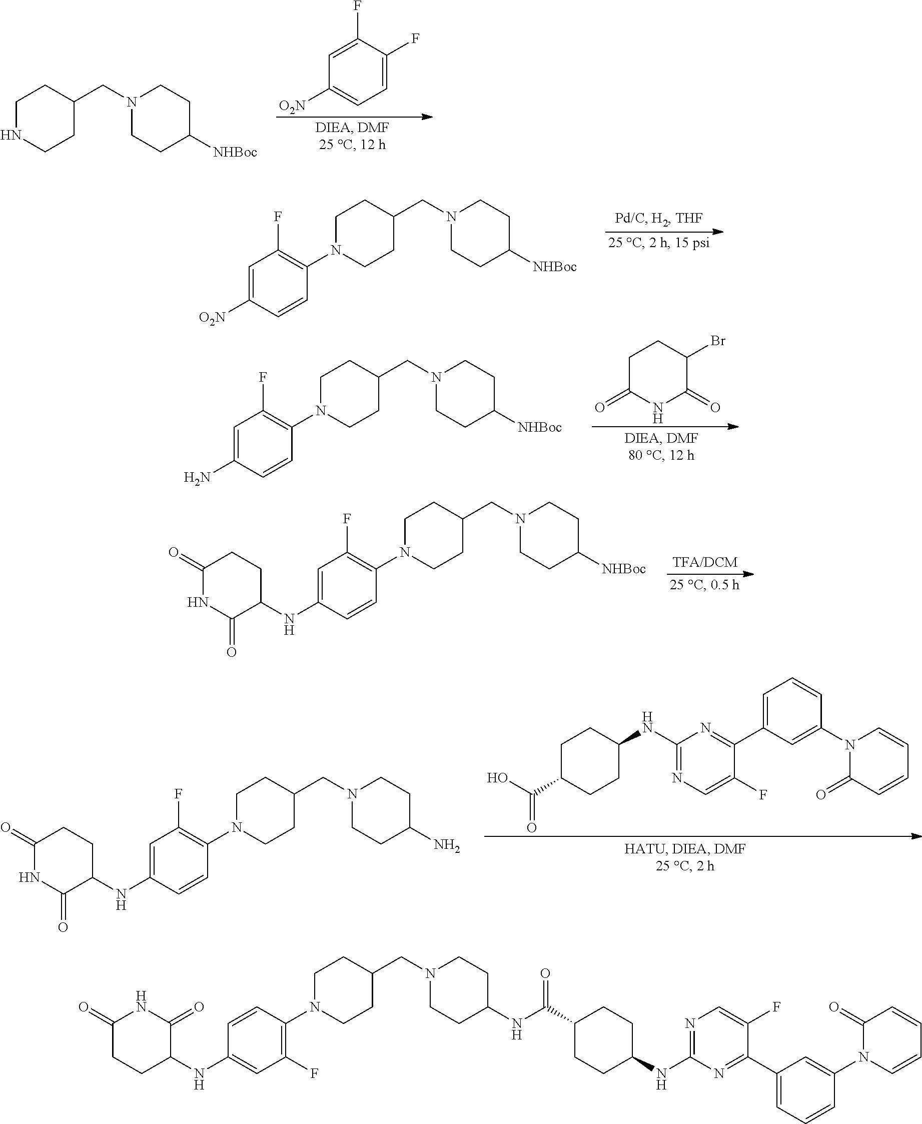

Synthesis of tert-butyl N-[1-[[1-(2-fluoro-4-nitro-phenyl)-4-piperidyl]methyl]-4-piperidyl]carbamate. To a solution of tert-butyl N-[1-(4-piperidylmethyl)-4-piperidyl]carbamate (5.80 g, 19.5 mmol, 1.00 eq) and 1,2-difluoro-4-nitro-benzene (3.10 g, 19.5 mmol, 2.16 mL, 1.00 eq) in DMF (70 mL) was added DIEA (7.56 g, 58.5 mmol, 10.2 mL, 3.00 eq) at 25 °C. The mixture was stirred at 25 °C for 12 h. LCMS showed that tert-butyl N-[1-(4-piperidylmethyl)-4-piperidyl]carbamate was consumed completely the desired mass was detected. H₂O (60 mL) was added to the solution, then solid precipitated, filtered and the filtered cake was dried under reduced pressure to give a residue. The residue was purified by MPLC (SiO₂, Petroleum ether/Ethyl acetate=1/0 to 1/1) to give the title compound (7.04 g, 16.1 mmol, 82.71% yield) as a yellow oil. LCMS (ES⁺): m/z 437.2 [M+H]⁺.

Synthesis of tert-butyl N-[1-[[1-(4-amino-2-fluoro-phenyl)-4-piperidyl]methyl]-4-piperidyl]carbamate. To a solution of tert-butyl N-[1-[[1-(2-fluoro-4-nitro-phenyl)-4-piperidyl]methyl]-4-piperidyl]carbamate (4.00 g, 9.16 mmol, 1.00 eq) in THF (40 mL) was added 10% Pd/C (8.00 g, 50% purity) at 25 °C, the mixture was degassed and purged with H₂ foe three times and the resulting mixture was stirred at 25 °C for 2 h under H₂ (15 psi). LCMS showed that tert-butyl N-[1-[[1-(2-fluoro-4-nitro-phenyl)-4-piperidyl]methyl]-4-piperidyl]carbamate was consumed completely and the desired mass was detected. The mixture was filtered and the filtrate was concentrated under reduced pressure to give the title compound (3.00 g, crude) as a yellow oil. LCMS (ES⁺): m/z 407.3 [M+H]⁺.

Synthesis of tert-butyl N-[1-[[1-[4-[(2,6-dioxo-3-piperidyl) amino]-2-fluoro-phenyl]-4-piperidyl]methyl]-4-piperidyl]carbamate. To a solution of tert-butyl N-[1-[[1-(4-amino-2-fluoro-phenyl)-4-piperidyl]methyl]-4-piperidyl]carbamate (2.40 g, 5.90 mmol, 1.00 eq) and 3-bromopiperidine-2,6-dione (2.27 g, 11.8 mmol, 2.00 eq) in DMF (30 mL) was added DIEA (2.29 g, 17.7 mmol, 3.08 mL, 3.00 eq) at 25 °C. After addition, the mixture was stirred at 80 °C for 12 h. LCMS showed that tert-butyl N-[1-[[1-(4-amino-2-fluoro-phenyl)-4-piperidyl]methyl]-4-piperidyl]carbamate was consumed completely and the desired mass was detected. H₂O (30 mL) was added to the solution, the mixture was extracted with EtOAc (30 mL*3), the combined organic layers were washed with brine (30 mL), then dried over Na₂SO₄, filtered and the filtrate was concentrated under reduced pressure to give a residue. The residue was purified by MPLC (SiO₂, Petroleum ether/Ethyl acetate=1/0 to 1/1) to give the title compound (300 mg, 580 μmol, 9.82% yield) as a brown solid. LCMS (ES⁺): m/z 518.3 [M+H]⁺.

Synthesis of 3-[4-[4-[(4-amino-1-piperidyl) methyl]-1-piperidyl]-3-fluoro-anilino]piperidine-2,6-dione. To a solution of tert-butyl N-[[1-[1-[4-[(2,6-dioxo-3-piperidyl) amino]-2-fluoro-phenyl]-4-piperidyl]methyl]-4-piperidyl]carbamate (300 mg, 580 μmol, 1.00 eq) in DCM (3 mL) was added dropwise TFA (1 mL) at 25 °C. After addition, the mixture was stirred at 25 °C for 0.5 h. LCMS showed that tert-butyl N-[1-[[1-[4-[(2,6-dioxo-3-piperidyl) amino]-2-fluoro-phenyl]-4-piperidyl]methyl]-4-piperidyl]carbamate was consumed completely and the desired mass was detected. The mixture was concentrated under reduced pressure to give the title compound (300 mg, crude, TFA) as a yellow oil. LCMS (ES⁺): m/z 418.2 [M+H]⁺.

Synthesis of trans-N-(1-((1-(4-((2,6-dioxopiperidin-3-yl)amino)-2-fluorophenyl)piperidin-4-yl)methyl)piperidin-4-yl)-4-((5-fluoro-4-(3-(2-oxopyridin-1(2H)-yl)phenyl)pyrimidin-2-yl)amino)cyclohexane-1-carboxamide (Compound 375). To a solution of trans-4-[[5-fluoro-4-[3-(2-oxo-1-pyridyl) phenyl]pyrimidin-2-yl]amino]cyclohexanecarboxylic acid (161 mg, 395 μmol, 0.700 eq) in DMF (5 mL) were added HATU (322 mg, 847 μmol, 1.50 eq) and DIEA (219 mg, 1.69 mmol, 295 μL, 3.00 eq) at 25 °C. And then 3-[4-[4-[(4-amino-1-piperidyl) methyl]-1-piperidyl]-3-fluoro-anilino]piperidine-2,6-dione (300 mg, 564 μmol, 1.00 eq, TFA) was added to the mixture at 25 °C. The resulting mixture was stirred at 25 °C for 2 h. LCMS showed that 3-[4-[4-[(4-amino-1-piperidyl) methyl]-1-piperidyl]-3-fluoro-anilino]piperidine-2,6-dione was consumed completely and the desired mass was detected. The mixture was concentrated under reduced pressure to give a residue. The residue was purified by prep-HPLC (column: Phenomenex Luna C18 75*30 mm*3 um; mobile phase: [H₂O (0.2% FA)-ACN]; gradient: 5%-45% B over 8.0 min) to give the title compound (Compound 375) (56.3 mg, 67.6 μmol, 12.0% yield, 97.1% purity) as a yellow solid. LCMS (ES⁺): m/s 808.4 [M+H]⁺

### Example 390. Synthesis of trans-N-((trans-4-((4-(4-((2,6-dioxopiperidin-3-yl)amino)-2-fluorophenyl)piperazin-1-yl)methyl)cyclohexyl)methyl)-4-((5-fluoro-4-(3-(2-oxopyridin-1(2H)-yl)phenyl)pyrimidin-2-yl)amino)cyclohexane-1-carboxamide (Compound 416)

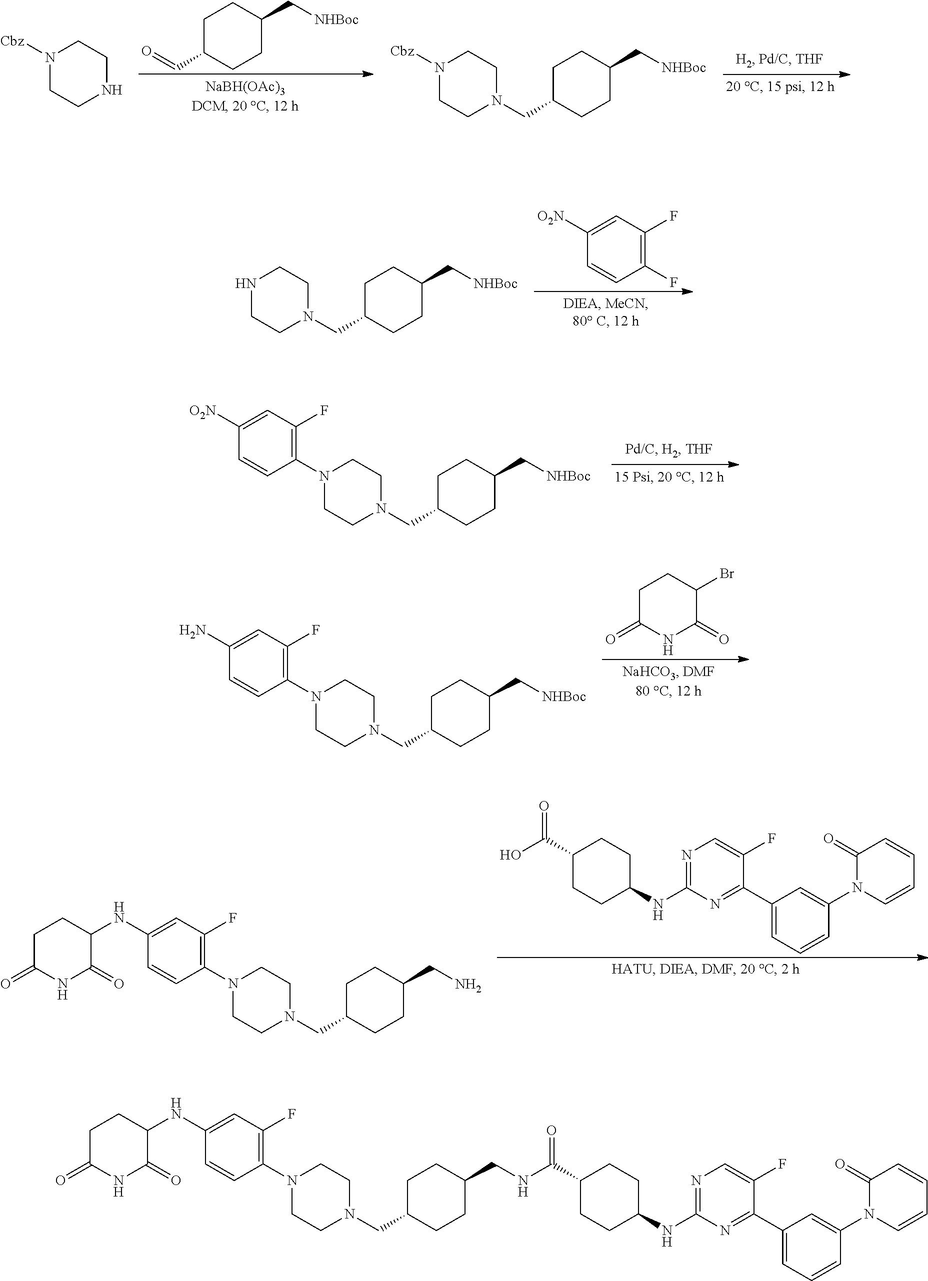

Synthesis of trans-benzyl 4-(((1r,4r)-4-(((tert-butoxycarbonyl)amino)methyl)cyclohexyl)methyl)piperazine-1-carboxylate. To a solution of benzyl piperazine-5-carboxylate (4.50 g, 20.4 mmol, 3.94 mL, 1.00 eq) and trans-tert-butyl (((1r,4r)-4-formylcyclohexyl)methyl)carbamate (4.93 g, 20.4 mmol, 1 eq) in DCM (50 mL) was added NaBH(OAc)₃ (10.8 g, 51.1 mmol, 2.50 eq) at 20 °C. The mixture was stirred at 20 °C for 12 h. LC-MS showed benzyl piperazine-1-carboxylate was consumed completely and desired mass was detected. The residue was diluted with H₂O (50 mL) and extracted with DCM (40 mL*3). The combined organic layers were washed with brine (60 mL), dried over Na₂SO₄, filtered and concentrated under reduced pressure to give a residue. The residue was purified by column chromatography (SiO₂, Petroleum ether/Ethyl acetate=1/0 to 0/1) to give the title compound (13.0 g, crude) as a white solid. LCMS (ES): m/z 446.2 [M+H]⁺.

Synthesis of trans-benzyl 4-(((1r,4r)-4-(((tert-butoxycarbonyl)amino)methyl)cyclohexyl)methyl)piperazine-lJ-carboxylate. To a mixture of 10% Pd/C (6.00 g, 50.0% purity) in THF (50 mL) was added trans-benzyl 4-(((1r,4r)-4-(((tert-butoxycarbonyl)amino)methyl)cyclohexyl)methyl)piperazine-1-carboxylate (13.0 g, 29.2 mmol, 1.00 eq) at 20 °C. The mixture was stirred at 20 °C for 12 h under H₂ (15 Psi). LC-MS showed that trans-benzyl 4-(((1r,4r)-4-(((tert-butoxycarbonyl)amino)methyl)cyclohexyl)methyl)piperazine-1-carboxylate was consumed completely and desired mass was detected. The mixture was filtered and filtrate was concentrated under reduced pressure to remove solvent to give the title compound (11.0 g, crude) as a colorless oil. LCMS (ES⁺): m/z 312.2 [M+H]⁺.

Synthesis of trans-tert-butyl (((1r,4r)-4-((4-(2-fluoro-4-nitrophenyl)piperazin-1-yl)methyl)cyclohexyl)methyl)carbamate. To a solution of trans-benzyl 4-(((1r,4r)-4-(((tert-butoxycarbonyl)amino)methyl)cyclohexyl)methyl)piperazine-1-carboxylate (11.0 g, 35.3 mmol, 1.00 eq) and 1,2-difluoro-4-nitrobenzene (5.62 g, 35.3 mmol, 3.91 mL, 1.00 eq) in ACN (3.00 mL) was added DIEA (13.7 g, 106 mmol, 18.5 mL, 3.00 eq) at 20 °C. The mixture was stirred at 80 °C for 12 h. LC-MS showed that trans-benzyl 4-(((1r,4r)-4-(((tert-butoxycarbonyl)amino)methyl)cyclohexyl)methyl)piperazine-1-carboxylate was consumed completely and desired mass was detected. The mixture was filtered and filtrate was concentrated under reduced pressure to remove solvent to give the title compound (10.00 g, crude) as a yellow solid. LCMS (ES⁺): m/z 451.2 [M+H]⁺.

Synthesis of trans-tert-butyl (((1r,4r)-4-((4-(4-amino-2-fluorophenyl)piperazin-1-yl)methyl)cyclohexyl)methyl)carbamate. To a mixture of 10% Pd/C (1.00 g, 50.0% purity) in THF (60 mL) was added trans-tert-butyl (((1r,4r)-4-((4-(2-fluoro-4-nitrophenyl)piperazin-1-yl)methyl)cyclohexyl)methyl)carbamate (10.0 g, 22.2 mmol, 1.00 eq) at 20 °C. The mixture was stirred at 20 °C for 12 h under H₂ (15 Psi). LC-MS showed that trans-tert-butyl (((1r,4r)-4-((4-(2-fluoro-4-nitrophenyl)piperazin-1-yl)methyl)cyclohexyl)methyl)carbamate was consumed completely and desired mass was detected. The mixture was filtered and filtrate was concentrated under reduced pressure to remove solvent to give the title compound (9.00 g, crude) as a red oil. LCMS (ES⁺): m/z 421.2 [M+H]⁺.

Synthesis of trans-3-((4-(4-(((1r,4r)-4-(aminomethyl)cyclohexyl)methyl)piperazin-1-yl)-3-fluorophenyl)amino)piperidine-2,6-dione. To a solution of trans-tert-butyl (((1r,4r)-4-((4-(4-amino-2-fluorophenyl)piperazin-1-yl)methyl)cyclohexyl)methyl)carbamate (9.00 g, 21.4 mmol, 1.00 eq) and 3-bromopiperidine-2,6-dione (28.8 g, 150 mmol, 7.00 eq) in DMF (50 mL) was added NaHCO₃ (4.49 g, 53.5 mmol, 2.08 mL, 2.50 eq) at 20 °C. The mixture was stirred at 80 °C for 12 h. LC-MS showed that trans-tert-butyl (((1r,4r)-4-((4-(4-amino-2-fluorophenyl)piperazin-1-yl)methyl)cyclohexyl)methyl)carbamate was consumed completely and desired de-Boc mass was detected. The residue was diluted with H₂O (50 mL) and extracted with EtOAc (50 mL*5). The combined organic layers were washed with brine (50 mL), dried over Na₂SO₄, filtered and concentrated under reduced pressure to give a residue. The residue was purified by prep-HPLC (column: Phenomenex luna c18 250 mm*100 mm*10 um; mobile phase: [H₂O (0.2% FA)-ACN]; gradient: 0%-12% B over 40.0 min) to give the title compound (3.00 g, 6.95 mmol, 32.5% yield) as a gray solid. LCMS (ES⁺): m/z 432.2 [M+H]⁺.

Synthesis of trans-N-((trans-4-((4-(4-((2,6-dioxopiperidin-3-yl)amino)-2-fluorophenyl)piperazin-1-yl)methyl)cyclohexyl)methyl)-4-((5-fluoro-4-(3-(2-oxopyridin-1(2H)-yl)phenyl)pyrimidin-2-yl)amino)cyclohexane-1-carboxamide (Compound 416). To a solution of trans-3-((4-(4-(((1r,4r)-4-(aminomethyl)cyclohexyl)methyl)piperazin-1-yl)-3-fluorophenyl)amino)piperidine-2,6-dione (1.00 g, 2.32 mmol, 1.00 eq) and trans-(1r,4r)-4-((5-fluoro-4-(3-(2-oxopyridin-1(2H)-yl)phenyl)pyrimidin-2-yl)amino)cyclohexane-1-carboxylic acid (946 mg, 2.32 mmol, 1.00 eq) in DMF (20 mL) were added HATU (1.76 g, 4.63 mmol, 2.00 eq) and DIEA (898 mg, 6.95 mmol, 1.21 mL, 3.00 eq) at 20 °C. The mixture was stirred at 20 °C for 2 h. LC-MS showed that rac-3-((4-(4-(((1r,4r)-4-(aminomethyl)cyclohexyl)methyl)piperazin-1-yl)-3-fluorophenyl)amino)piperidine-2,6-dione was consumed completely and desired mass was detected. The residue was diluted with H₂O (20 mL) and extracted with EtOAc (20 mL*3). The combined organic layers were washed with brine 20 mL, dried over Na₂SO₄, filtered and concentrated under reduced pressure. The residue was purified by prep-HPLC (column: Phenomenex luna C18 100*40 mm*5 um; mobile phase: [H₂O (0.2% FA)-ACN]; gradient: 10%-40% B over 8.0 min) to give the title compound (Compound 416) (428 mg, 482 μmol, 20.8% yield, 94.1% purity, 0.3 FA) as a brown solid. LCMS (ES⁺): m/z 822.4 [M+H]⁺.

### Example 391. Synthesis of (1r,4R)—N-(((1R,4R)-4-((4-(4-(((R)-2,6-dioxopiperidin-3-yl)amino)-2-fluorophenyl)piperazin-1-yl)methyl)cyclohexyl)methyl)-4-((5-fluoro-4-(3-(2-oxopyridin-1(2H)-yl)phenyl)pyrimidin-2-yl)amino)cyclohexane-1-carboxamide (Compound 459) and (1r,4R)—N-(((1S,4R)-4-((4-(4-(((S)-2,6-dioxopiperidin-3-yl)amino)-2-fluorophenyl)piperazin-1-yl)methyl)cyclohexyl)methyl)-4-((5-fluoro-4-(3-(2-oxopyridin-1(2H)-yl)phenyl)pyrimidin-2-yl)amino)cyclohexane-1-carboxamide (Compound 460) (The Two Isomers were Assigned Arbitrarily)

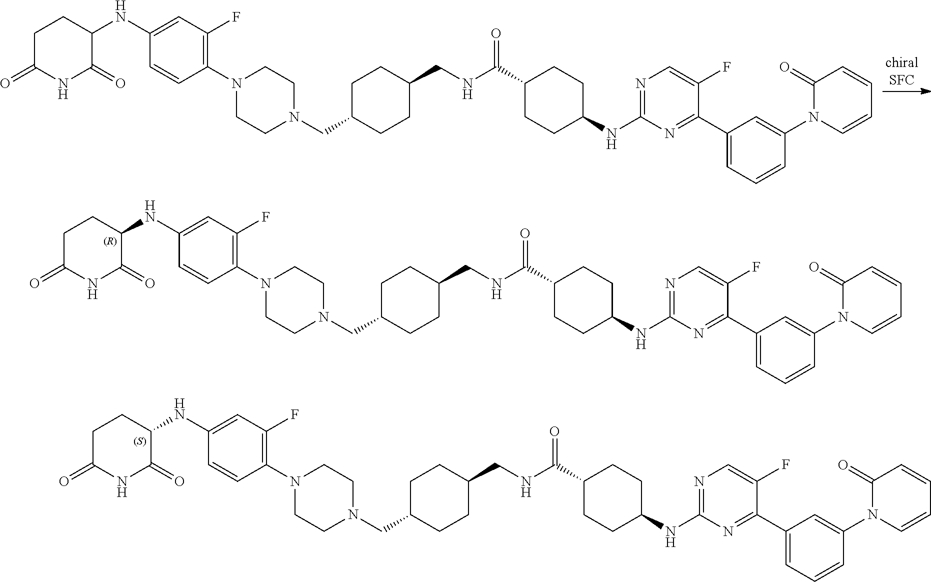

Synthesis of (1r,4R)—N-(((1R,4R)-4-((4-(4-(((R)-2,6-dioxopiperidin-3-yl)amino)-2-fluorophenyl)piperazin-1-yl)methyl)cyclohexyl)methyl)-4-((5-fluoro-4-(3-(2-oxopyridin-1(2H)-yl)phenyl)pyrimidin-2-yl)amino)cyclohexane-1-carboxamide (Compound 459) and (1r,4R)—N-(((1S,4R)-4-((4-(4-(((S)-2,6-dioxopiperidin-3-yl)amino)-2-fluorophenyl)piperazin-1-yl)methyl)cyclohexyl)methyl)-4-((5-fluoro-4-(3-(2-oxopyridin-1(2H)-yl)phenyl)pyrimidin-2-yl)amino)cyclohexane-1-carboxamide (Compound 460). rac-(1r,4R)—N-(((1r,4R)-4-((4-(4-((2,6-dioxopiperidin-3-yl)amino)-2-fluorophenyl)piperazin-1-yl)methyl)cyclohexyl)methyl)-4-((5-fluoro-4-(3-(2-oxopyridin-1(2H)-yl)phenyl)pyrimidin-2-yl)amino)cyclohexane-1-carboxamide (Compound 416) (200 mg, 228.97 μmol, 9.88% yield, 94.1% purity) was separated by chiral SFC (column: DAICEL CHIRALPAK IE (250 mm*30 mm, 10 um); mobile phase: [Heptane-IPA/CAN=4/1 (0.1% NH₃H₂O)]; B %: 100%, isocratic elution mode) to give isomer 1 (Compound 459) (57.6 mg, 68.46 μmol, 28.13% yield, 97.69% purity) as an off-white solid. LCMS (ES⁺): m/z 822.4 [M+H]⁺ and isomer 2 (Compound 460) (65.7 mg, 75.82 μmol, 31.16% yield, 94.86% purity) as a yellow solid. LCMS (ES⁺): m/z 822.3 [M+H]⁺.

### Example 392. Synthesis of trans-(1r,4R)—N-((1r,4R)-4-(4-(4-((2,6-dioxopiperidin-3-yl)amino)-2-fluorophenyl)piperidin-1-yl)cyclohexyl)-4-((5-fluoro-4-(3-(2-oxopyridin-1(2H)-yl)phenyl)pyrimidin-2-yl)amino)cyclohexane-1-carboxamide (Compound 401)

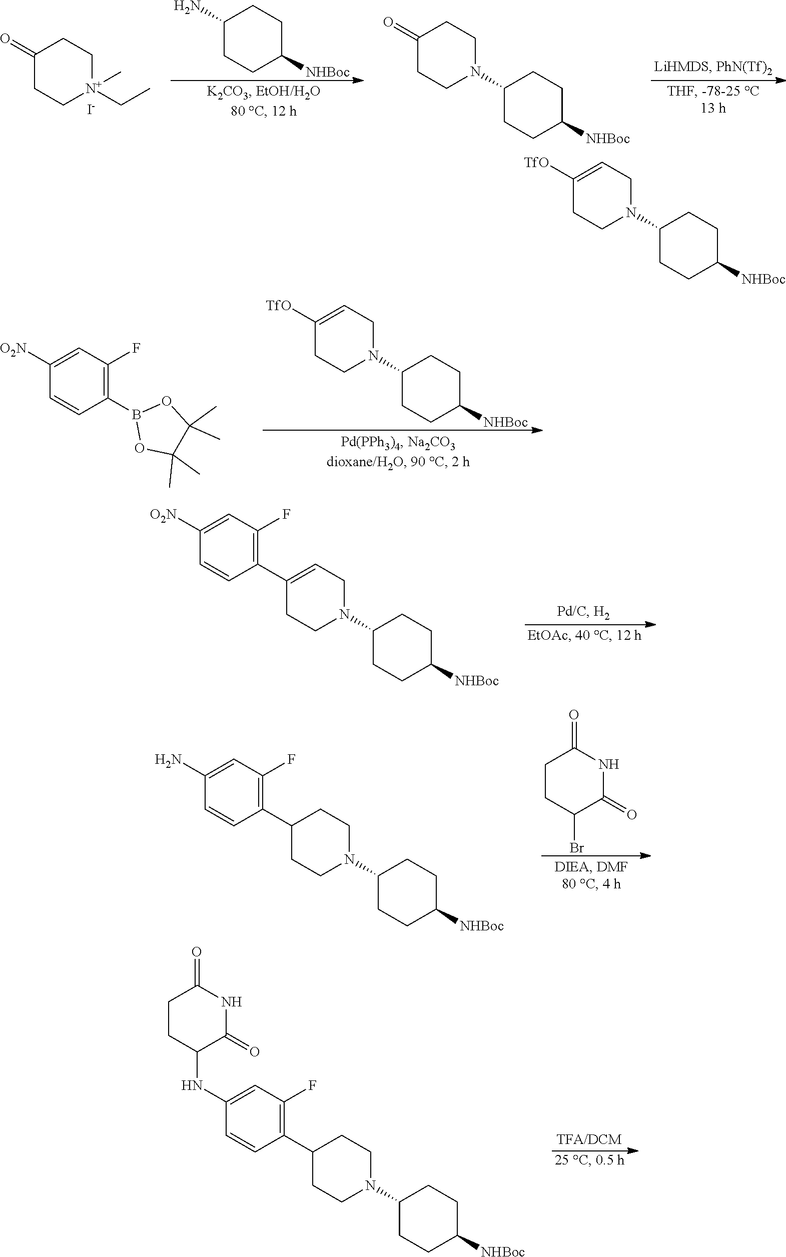

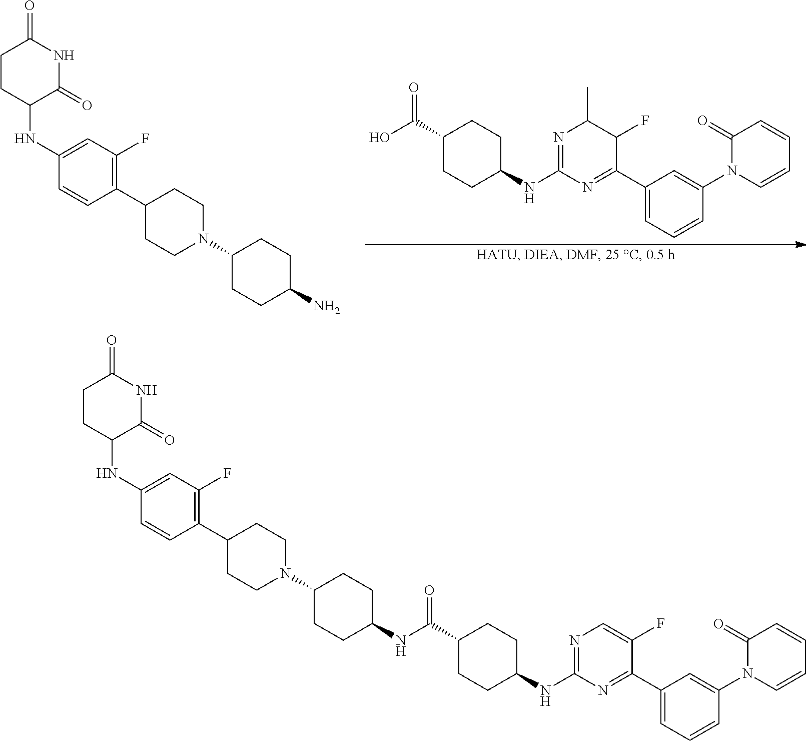 [SMILES: N[C@H]1CC[C@H](N2CCC(c3ccc(NC4CCC(=O)NC4=O)cc3F)CC2)CC1 ; CC1N=C(N[C@H]2CC[C@H](C(=O)O)CC2)N=C(c2cccc(-n3ccccc3=O)c2)C1F ; O=C1CCC(Nc2ccc(C3CCN([C@H]4CC[C@H](NC(=O)[C@H]5CC[C@H](Nc6ncc(F)c(-c7cccc(-n8ccccc8=O)c7)n6)CC5)CC4)CC3)c(F)c2)C(=O)N1]

Synthesis of trans-tert-butyl (4-(4-oxopiperidin-1-yl)cyclohexyl)carbamate. To a solution of 1-ethyl-1-methyl-4-oxopiperidin-1-ium iodide (12.0 g, 84.4 mmol, 1.00 eq) and trans-tert-butyl N-(4-aminocyclohexyl)carbamate (14.5 g, 67.5 mmol, 0.800 eq) in H₂O (131 mL) and EtOH (210 mL) was added K₂CO₃ (11.7 g, 84.4 mmol, 1.00 eq) at 25 °C. The mixture was stirred at 80 °C for 12 h. LC-MS showed 1-ethyl-1-methyl-4-oxopiperidin-1-ium iodide was consumed completely and the desired mass was detected. The reaction mixture was concentrated under reduced pressure to remove EtOH. Then Water (100 mL) was added to the aqueous phase at 25 °C. Then it was extracted with DCM (100 mL*3). The organic layers were dried over anhydrous sodium sulfate, filtered and the filtrate was concentrated under reduced pressure to give a residue. The residue was purified by column chromatography (SiO₂, Petroleum ether/Ethyl acetate=1/1 to 0/1) to give the title compound (10.0 g, 33.7 mmol, 40.0% yield) as a white solid. LCMS (ES⁺): m/z 297.2 [M+H]⁺

Synthesis of trans-1-(4-((tert-butoxycarbonyl)amino)cyclohexyl)-1,2,3,6-tetrahydropyridin-4-yl trifluoromethanesulfonate. To a solution of trans-tert-butyl (4-(4-oxopiperidin-1-yl)cyclohexyl)carbamate (10.0 g, 33.7 mmol, 1.00 eq) in THF (400 mL) was added LiHMDS (1 M solution in THF, 67.5 mL, 2.00 eq) at −78 °C under N₂ atmosphere. The mixture was stirred at −78 °C for 1 h under N₂. Then a solution of 1,1,1-trifluoro-N-phenyl-N-(trifluoromethylsulfonyl)methanesulfonamide (18.1 g, 50.6 mmol, 1.50 eq) in THF (30 mL) was added to the mixture at −78 °C under N₂. The mixture was slowly warmed to 25 °C and stirred at 25 °C for 12 h. LC-MS showed trans-tert-butyl (4-(4-oxopiperidin-1-yl)cyclohexyl)carbamate was consumed completely and the desired mass was detected. Saturated NH₄Cl aqueous solution (400 mL) was added to the mixture dropwise to quench the reaction at 0 °C. Then the above mixture was extracted with EtOAc (200 mL*3). The organic layers were dried over anhydrous sodium sulfate, filtered and the filtrate was concentrated under reduced pressure to give a residue. The residue was purified by MPLC (SiO₂, Petroleum ether:Ethyl acetate=1:0 to 1:1) to give the title compound (6.00 g, 14.0 mmol, 41.5% yield) as a white solid. LCMS (ES⁺): m/z 429.2 [M+H]⁺

Synthesis of trans-tert-butyl (4-(4-(2-fluoro-4-nitrophenyl)-3,6-dihydropyridin-1(2H)-yl)cyclohexyl)carbamate. To a solution of 2-(2-fluoro-4-nitrophenyl)-4,4,5,5-tetramethyl-1,3,2-dioxaborolane (3.86 g, 14.5 mmol, 1.00 eq) in dioxane (100 mL) and H₂O (10 mL) were added trans-1-(4-((tert-butoxycarbonyl)amino)cyclohexyl)-1,2,3,6-tetrahydropyridin-4-yl trifluoromethanesulfonate (6.20 g, 14.5 mmol, 1.00 eq), Pd(PPh₃)₄ (334 mg, 289 μmol, 0.0200 eq) and Na₂CO₃ (3.07 g, 29.0 mmol, 2.00 eq) at 25 °C under N₂ atmosphere. The mixture was stirred at 90 °C for 2 h under N₂. LC-MS showed 2-(2-fluoro-4-nitrophenyl)-4,4,5,5-tetramethyl-1,3,2-dioxaborolane was consumed completely and the desired mass was detected. Water (60 mL) was added to the mixture at 20 °C. Then the reaction mixture was extracted with EtOAc (50 mL*3). The organic layers were dried over anhydrous sodium sulfate, filtered and the filtrate was concentrated under reduced pressure to give a residue. The residue was purified by column chromatography (SiO₂, Petroleum ether/Ethyl acetate=3/1 to 1/1) to give the title compound (4.90 g, 11.7 mmol, 80.7% yield) as a white solid. LCMS (ES⁺): m/z 420.3 [M+H]⁺

Synthesis of trans-tert-butyl (4-(4-(4-amino-2-fluorophenyl)piperidin-1-yl)cyclohexyl)carbamate. 10% Pd/C (3.00 g, 50% purity) was added to EtOAc (100 mL) at 25 °C under N₂ atmosphere, then trans-tert-butyl (4-(4-(2-fluoro-4-nitrophenyl)-3,6-dihydropyridin-1(2H)-yl)cyclohexyl)carbamate (4.90 g, 11.7 mmol, 1.00 eq) was added to the mixture at 25 °C, the suspension was degassed and purged with H₂ for three times. The mixture was stirred at 40 °C for 12 h under H₂ (15 psi) atmosphere. LC-MS showed trans-tert-butyl (4-(4-(2-fluoro-4-nitrophenyl)-3,6-dihydropyridin-1(2H)-yl)cyclohexyl)carbamate was consumed completely and the desired mass was detected. The mixture was filtered and the filtrate was concentrated under reduced pressure to give the title compound (4.50 g, crude) as a white solid. LCMS (ES⁺): m/z 392.3 [M+H]⁺

Synthesis of trans-tert-butyl (4-(4-(4-((2,6-dioxopiperidin-3-yl)amino)-2-fluorophenyl)piperidin-1-yl)cyclohexyl)carbamate. Four batches in parallel were set up. To a solution of trans-tert-butyl (4-(4-(4-amino-2-fluorophenyl)piperidin-1-yl)cyclohexyl)carbamate (1.00 g, 2.55 mmol, 1.00 eq) in DMF (20 mL) were added DIEA (990 mg, 7.66 mmol, 1.33 mL, 3.00 eq) and 3-bromopiperidine-2,6-dione (1.47 g, 7.66 mmol, 3.00 eq) at 25 °C. The mixture was stirred at 80 °C for 2 h. LC-MS showed trans-tert-butyl (4-(4-(4-amino-2-fluorophenyl)piperidin-1-yl)cyclohexyl)carbamate was remaining and the desired mass was detected. Then 3-bromopiperidine-2,6-dione (1.47 g, 7.66 mmol, 3.00 eq) was added to the mixture at 25 °C. The mixture was stirred at 80 °C for another 2 h. LC-MS showed trans-tert-butyl (4-(4-(4-amino-2-fluorophenyl)piperidin-1-yl)cyclohexyl)carbamate was consumed completely and the desired mass was detected. Four batches reaction were combined for work up. Water (80 mL) was added to the mixture at 25 °C. Then the reaction mixture was extracted with EtOAc (80 mL*3). The organic layers were dried over anhydrous sodium sulfate, filtered and the filtrate was concentrated under reduced pressure to give a residue. The residue was purified by column chromatography (SiO₂, Petroleum ether/Ethyl acetate=1/1 to 1/2) to give the title compound (1.50 g, 2.98 mmol, 29.2% yield) as a brown solid. LCMS (ES⁺): m/z 503.4 [M+H]⁺

Synthesis of trans-3-((4-(1-(4-aminocyclohexyl)piperidin-4-yl)-3-fluorophenyl)amino)piperidine-2,6-dione. A solution of trans-tert-butyl (4-(4-(4-((2,6-dioxopiperidin-3-yl)amino)-2-fluorophenyl)piperidin-1-yl)cyclohexyl)carbamate (600 mg, 1.19 mmol, 1.00 eq) in TFA (2 mL) and DCM (6 mL) was stirred at 25 °C for 0.5 h. LCMS showed trans-tert-butyl (4-(4-(4-((2,6-dioxopiperidin-3-yl)amino)-2-fluorophenyl)piperidin-1-yl)cyclohexyl)carbamate was consumed completely and the desired mass was detected. The reaction mixture was concentrated under reduced pressure to give the title compound (610 mg, crude) as a yellow oil. LCMS (ES⁺): m/z 403.4 [M+H]⁺

Synthesis of trans-(1r,4R)—N-((1r,4R)-4-(4-(4-((2,6-dioxopiperidin-3-yl)amino)-2-fluorophenyl)piperidin-1-yl)cyclohexyl)-4-((5-fluoro-4-(3-(2-oxopyridin-1(2H)-yl)phenyl)pyrimidin-2-yl)amino)cyclohexane-1-carboxamide (Compound 401). To a solution of trans-3-((4-(1-(4-aminocyclohexyl)piperidin-4-yl)-3-fluorophenyl)amino)piperidine-2,6-dione (600 mg, 1.49 mmol, 1 eq) and 4-[[5-fluoro-4-[3-(2-oxo-1-pyridyl)phenyl]pyrimidin-2-yl]amino]cyclohexanecarboxylic acid (426 mg, 1.04 mmol, 0.700 eq) in DMF (5 mL) were added HATU (567 mg, 1.49 mmol, 1.00 eq) and DIEA (578 mg, 4.47 mmol, 779 μL, 3.00 eq) at 25 °C, the mixture was stirred at 25 °C for 0.5 hr. LCMS showed trans-3-((4-(1-(4-aminocyclohexyl)piperidin-4-yl)-3-fluorophenyl)amino)piperidine-2,6-dione was consumed completely and the desired mass was detected. The reaction was filtered and the filtrate was concentrated under reduced pressure to give a residue. The residue was purified by prep-HPLC (column: Phenomenex Luna C18 75*30 mm*3 um; mobile phase: [water (0.04% HCl)-ACN]; gradient: 10%-50% B over 8.0 min) to give the title compound (Compound 401) (45.0 mg, 54.8 μmol, 3.68% yield, 96.6% purity) as a yellow solid. LCMS (ES⁺): m/z 793.4 [M+H]⁺

### Example 393. Synthesis of trans-(1r,4R)—N-((1R,4R)-4-(4-(4-(((R)-2,6-dioxopiperidin-3-yl)amino)-2-fluorophenyl)piperidin-1-yl)cyclohexyl)-4-((5-fluoro-4-(3-(2-oxopyridin-1(2H)-yl)phenyl)pyrimidin-2-yl)amino)cyclohexane-1-carboxamide (Compound 418) and trans-(1r,4R)—N-((1S,4R)-4-(4-(4-(((S)-2,6-dioxopiperidin-3-yl)amino)-2-fluorophenyl)piperidin-1-yl)cyclohexyl)-4-((5-fluoro-4-(3-(2-oxopyridin-1(2H)-yl)phenyl)pyrimidin-2-yl)amino)cyclohexane-1-carboxamide (Compound 434) (The Two Isomers were Assigned Arbitrarily)

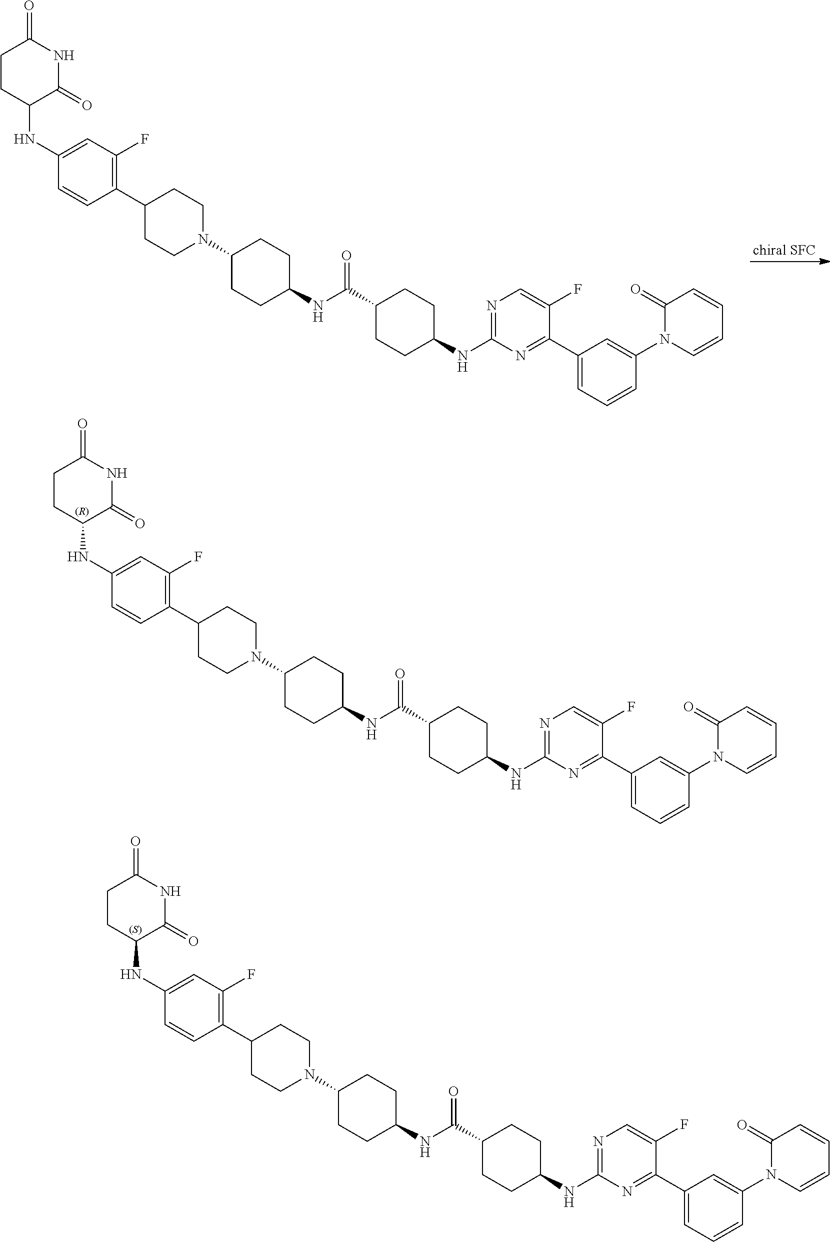

Synthesis of trans-(1r,4R)—N-((1R,4R)-4-(4-(4-(((R)-2,6-dioxopiperidin-3-yl)amino)-2-fluorophenyl)piperidin-1-yl)cyclohexyl)-4-((5-fluoro-4-(3-(2-oxopyridin-1(2H)-yl)phenyl)pyrimidin-2-yl)amino)cyclohexane-1-carboxamide (Compound 418) and trans-(1r,4R)—N-((1S,4R)-4-(4-(4-(((S)-2,6-dioxopiperidin-3-yl)amino)-2-fluorophenyl)piperidin-1-yl)cyclohexyl)-4-((5-fluoro-4-(3-(2-oxopyridin-1(2H)-yl)phenyl)pyrimidin-2-yl)amino)cyclohexane-1-carboxamide (Compound 434) (the two isomers were assigned arbitrarily). trans-(1r,4R)—N-((1r,4R)-4-(4-(4-((2,6-dioxopiperidin-3-yl)amino)-2-fluorophenyl)piperidin-1-yl)cyclohexyl)-4-((5-fluoro-4-(3-(2-oxopyridin-1(2H)-yl)phenyl)pyrimidin-2-yl)amino)cyclohexane-1-carboxamide (Compound 401) (50 mg, 63.1 umol, 1.00 eq) was separated by chiral SFC (column: REGIS(S,S)WHELK-O1 (250 mm*25 mm, 10 um); mobile phase: [CO₂-IPA (0.1% IPAm)]; B %: 56%, isocratic elution mode) to give the title compound (Compound 418) (4.00 mg, 5.04 μmol, 8.00% yield, 93.5% purity, 100% ee) (retention time: 2.220, peak 1) as a white solid. LCMS (ES+): m/z 793.3 [M+H]⁺ and the title compound (Compound 434) (6.60 mg, 8.32 μmol, 13.2% yield, 92.3% purity, 100% ee) (retention time: 2.868, peak 2) as a yellow solid. LCMS (ES+): m/z 793.3 [M+H]⁺.

### Example 394. Synthesis of trans-(1r,4R)—N-((1r,4R)-4-(4-(4-((2,6-dioxopiperidin-3-yl)amino)-2-fluorophenyl)piperazin-1-yl)cyclohexyl)-4-((5-fluoro-4-(3-(2-oxopyridin-1(2H)-yl)phenyl)pyrimidin-2-yl)amino)cyclohexane-1-carboxamide (Compound 381)

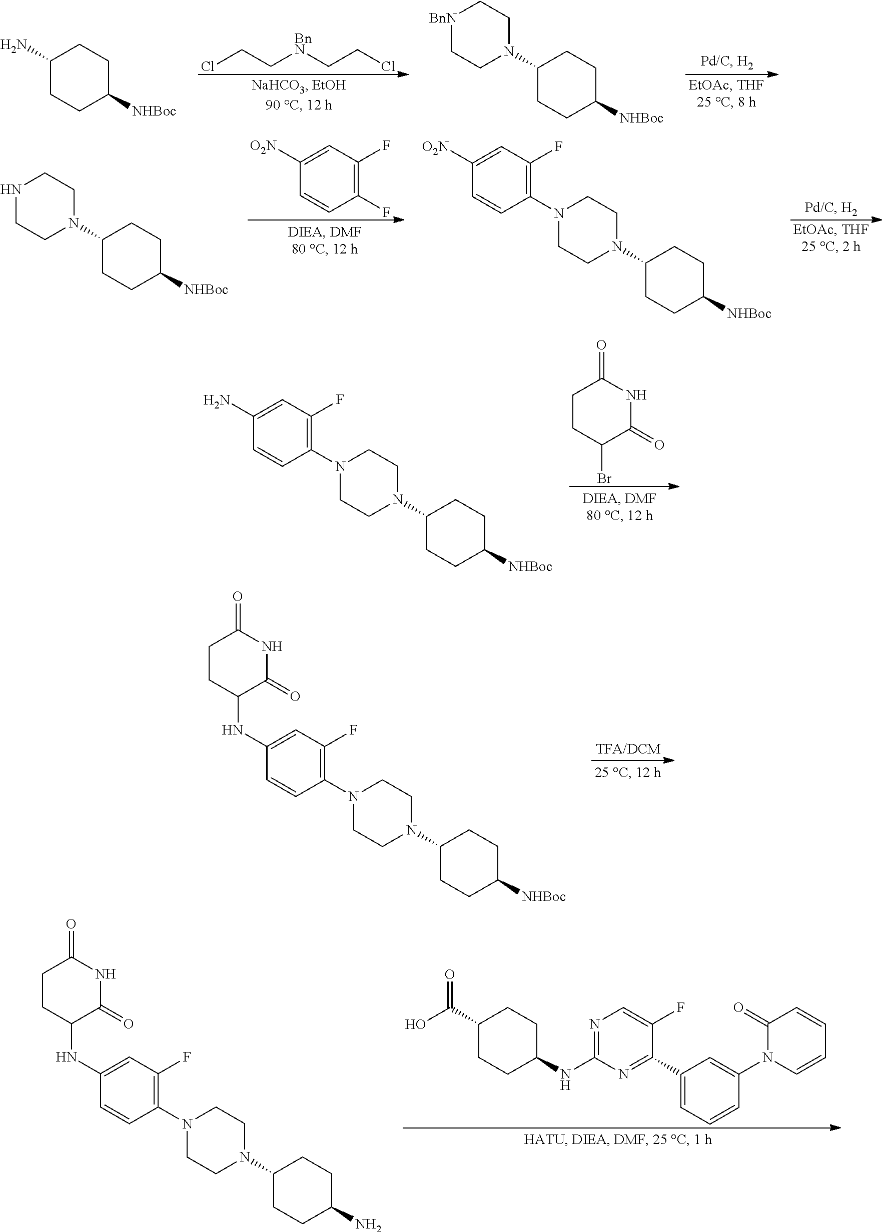

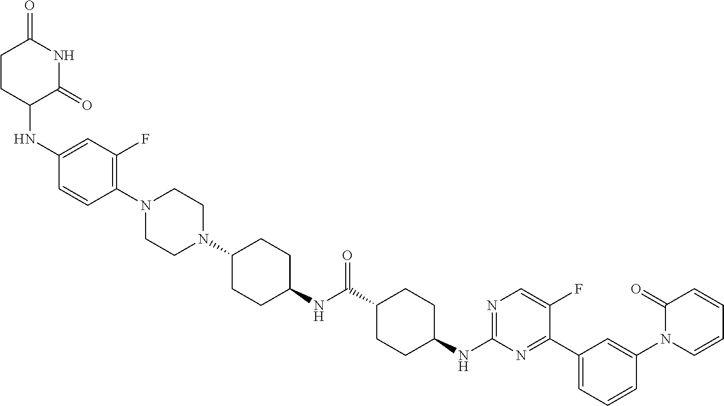 [SMILES: O=C1CCC(Nc2ccc(N3CCN([C@H]4CC[C@H](NC(=O)[C@H]5CC[C@H](Nc6ncc(F)c(-c7cccc(-n8ccccc8=O)c7)n6)CC5)CC4)CC3)c(F)c2)C(=O)N1]

Synthesis of trans-tert-butyl ((1r,4r)-4-(4-benzylpiperazin-1-yl)cyclohexyl)carbamate. To a solution of trans-tert-butyl ((1r,4r)-4-aminocyclohexyl)carbamate (4.62 g, 21.5 mmol, 1.00 eq) in EtOH (90 mL) were added N-benzyl-2-chloro-N-(2-chloroethyl)ethanamine (5.00 g, 21.5 mmol, 1.00 eq) and NaHCO₃ (4.52 g, 53.8 mmol, 2.10 mL, 2.50 eq) at 25 °C. The mixture was stirred at 90 °C for 12 h. LCMS showed trans-tert-butyl ((1r,4r)-4-aminocyclohexyl)carbamate was remaining little, and the desired mass was detected. TLC (SiO₂, Petroleum ether:Ethyl acetate=0:1) showed trans-tert-butyl ((1r,4r)-4-aminocyclohexyl)carbamate was remaining little, and three new spots formed. The reaction mixture was filtered with DCM (100 mL) and THF (100 mL) and the filtrate was concentrated under reduced to give a residue. The residue was purified by column chromatography (SiO₂, Petroleum ether:Ethyl acetate=2:1 to 0:1) to give the title compound (4.52 g, 12.1 mmol, 56.2% yield) as a white solid. LCMS (ES⁺): m/z 374.2 [M+H]⁺.

Synthesis of trans-tert-butyl ((1r,4r)-4-(piperazin-1-yl)cyclohexyl)carbamate. 10% Pd/C (13.0 g, 50% purity) was added to EtOAc (100 mL) and THF (200 mL) at 25 °C under N₂ atmosphere, then trans-tert-butyl ((1r,4r)-4-(4-benzylpiperazin-1-yl)cyclohexyl)carbamate (4.20 g, 11.2 mmol, 1.00 eq) was added to the mixture at 25 °C, the suspension was degassed and purged with H₂ for three times. The mixture was stirred at 25 °C for 8 h under H₂ (15 psi) atmosphere. LC-MS showed trans-tert-butyl ((1r,4r)-4-(4-benzylpiperazin-1-yl)cyclohexyl)carbamate was consumed, and the desired mass was detected. The reaction mixture was filtered with THF (1000 mL). The filtrate was concentrated under reduced pressure to give the title compound (5.23 g, crude) as a white solid. LCMS (ES⁺): m/z 284.2 [M+H]⁺.

Synthesis of trans-tert-butyl ((1r,4r)-4-(4-(2-fluoro-4-nitrophenyl)piperazin-1-yl)cyclohexyl)carbamate. To a solution of trans-tert-butyl ((1r,4r)-4-(piperazin-1-yl)cyclohexyl)carbamate (2.45 g, 8.64 mmol, 1.00 eq) in DMF (50 mL) were added 1,2-difluoro-4-nitrobenzene (1.38 g, 8.64 mmol, 957 μL, 1.00 eq) and DIEA (2.23 g, 17.3 mmol, 3.01 mL, 2.00 eq) at 25 °C. The mixture was stirred at 80 °C for 12 h. LCMS showed trans-tert-butyl ((1r,4r)-4-(piperazin-1-yl)cyclohexyl)carbamate was consumed completely and the desired mass was detected. H₂O (50 mL) was added to the reaction mixture at 25 °C, then it was filtered. The filter cake was dried under reduced pressure to give the title compound (5.28 g, crude) as a brown solid. LCMS (ES⁺): m/z 423.3 [M+H]⁺.

Synthesis of trans-tert-butyl ((1r,4r)-4-(4-(4-amino-2-fluorophenyl)piperazin-1-yl)cyclohexyl)carbamate. 10% Pd/C (9.60 g, 50% purity) was added to EtOAc (70 mL) and THF (30 mL) at 25 °C under N₂ atmosphere, then trans-tert-butyl ((1r,4r)-4-(4-(2-fluoro-4-nitrophenyl)piperazin-1-yl)cyclohexyl)carbamate (4.78 g, 11.3 mmol, 1.00 eq) was added to the mixture at 25 °C, the suspension was degassed and purged with H₂ for three times. The mixture was stirred at 25 °C for 2 h under H₂ (15 psi) atmosphere. LCMS showed trans-tert-butyl ((1r,4r)-4-(4-(2-fluoro-4-nitrophenyl)piperazin-1-yl)cyclohexyl)carbamate was consumed completely and the desired mass was detected. The reaction mixture was filtered with EtOAc (1000 mL). The filtrate was concentrated under reduced pressure to give the title compound (3.46 g, crude) as a yellow solid. LCMS (ES⁺): m/z 393.3 [M+H]⁺.

Synthesis of trans-tert-butyl ((1r,4r)-4-(4-(4-((2,6-dioxopiperidin-3-yl)amino)-2-fluorophenyl)piperazin-1-yl)cyclohexyl)carbamate. To a solution of trans-tert-butyl ((1r,4r)-4-(4-(4-amino-2-fluorophenyl)piperazin-1-yl)cyclohexyl)carbamate (3.10 g, 7.90 mmol, 1.00 eq) in DMF (60 mL) were added DIEA (3.06 g, 23.7 mmol, 4.13 mL, 3.00 eq) and 3-bromopiperidine-2,6-dione (3.03 g, 15.8 mmol, 2.00 eq) at 25 °C. The mixture was stirred at 80 °C for 12 h. LC-MS showed trans-tert-butyl ((1r,4r)-4-(4-(4-amino-2-fluorophenyl)piperazin-1-yl)cyclohexyl)carbamate was consumed completely, and the desired mass was detected. Water (60 mL) was added to the mixture at 25 °C. The reaction mixture was extracted with DCM (30 mL*5). The organic layers were dried over anhydrous sodium sulfate, filtered and the filtrate was concentrated under reduced to give a residue. The residue was purified by column chromatography (SiO₂, Petroleum ether:Ethyl acetate=2:1 to 0:1) to give the title compound (3.30 g, 6.55 mmol, 83.0% yield) as a brown solid. LCMS (ES⁺): m/z 504.3 [M+H]⁺.

Synthesis of trans-3-((4-(4-((1r,4r)-4-aminocyclohexyl)piperazin-1-yl)-3-fluorophenyl)amino)piperidine-2,6-dione. To a solution of trans-tert-butyl ((1r,4r)-4-(4-(4-((2,6-dioxopiperidin-3-yl)amino)-2-fluorophenyl)piperazin-1-yl)cyclohexyl)carbamate (2.00 g, 3.97 mmol, 1.00 eq) in DCM (12 mL) was added TFA (6.14 g, 53.9 mmol, 4.00 mL, 13.6 eq) at 25 °C. The mixture was stirred at 25 °C for 12 h. LCMS showed trans-tert-butyl ((1r,4r)-4-(4-(4-((2,6-dioxopiperidin-3-yl)amino)-2-fluorophenyl)piperazin-1-yl)cyclohexyl)carbamate was consumed completely and the desired mass was detected. The reaction mixture was concentrated under reduced pressure to give the title compound (2.06 g, crude, TFA) as a yellow oil. LCMS (ES⁺): m/z 404.2 [M+H]⁺.

Synthesis of trans-(1r,4R)—N-((r,4R)-4-(4-(4-((2,6-dioxopiperidin-3-yl)amino)-2-fluorophenyl)piperazin-1-yl)cyclohexyl)-4-((5-fluoro-4-(3-(2-oxopyridin-1(2H)-yl)phenyl)pyrimidin-2-yl)amino)cyclohexane-1-carboxamide (Compound 381). To a solution of trans-(1r,4r)-4-((5-fluoro-4-(3-(2-oxopyridin-1(2H)-yl)phenyl)pyrimidin-2-yl)amino)cyclohexane-1-carboxylic acid (395 mg, 966 μmol, 0.500 eq) in DMF (7 mL) were added HATU (808 mg, 2.13 mmol, 1.10 eq), DIEA (749 mg, 5.80 mmol, 1.01 mL, 3.00 eq) and trans-3-((4-(4-((1r,4r)-4-aminocyclohexyl)piperazin-1-yl)-3-fluorophenyl)amino)piperidine-2,6-dione (1.00 g, 1.93 mmol, 1.00 eq, TFA) at 25 °C. The mixture was stirred at 25 °C for 1 h. LCMS showed trans-3-((4-(4-((1r,4r)-4-aminocyclohexyl)piperazin-1-yl)-3-fluorophenyl)amino)piperidine-2,6-dione was consumed completely and the desired mass was detected. The reaction mixture was concentrated under reduced pressure to remove solvent. The residue was purified by prep-HPLC (column: Waters Xbridge BEH C18 250*50 mm*10 um; mobile phase: [H₂O (10 mM NH₄HCO₃)-ACN]; gradient: 25%-55% B over 10.0 min) to give the title compound (Compound 381) (72.1 mg, 87.1 μmol, 4.51% yield, 95.9% purity) as a yellow solid. LCMS (ES⁺): m/z 794.2 [M+H]⁺

### Example 395. Synthesis of trans-(1r,4R)—N-((1R,4R)-4-(4-(4-(((R)-2,6-dioxopiperidin-3-yl)amino)-2-fluorophenyl)piperazin-1-yl)cyclohexyl)-4-((5-fluoro-4-(3-(2-oxopyridin-1(2H)-yl)phenyl)pyrimidin-2-yl)amino)cyclohexane-1-carboxamide (Compound 420) and trans-(1r,4R)—N-((1S,4R)-4-(4-(4-(((S)-2,6-dioxopiperidin-3-yl)amino)-2-fluorophenyl)piperazin-1-yl)cyclohexyl)-4-((5-fluoro-4-(3-(2-oxopyridin-1(2H)-yl)phenyl)pyrimidin-2-yl)amino)cyclohexane-1-carboxamide (Compound 436) (The Two Isomers were Assigned Arbitrarily)

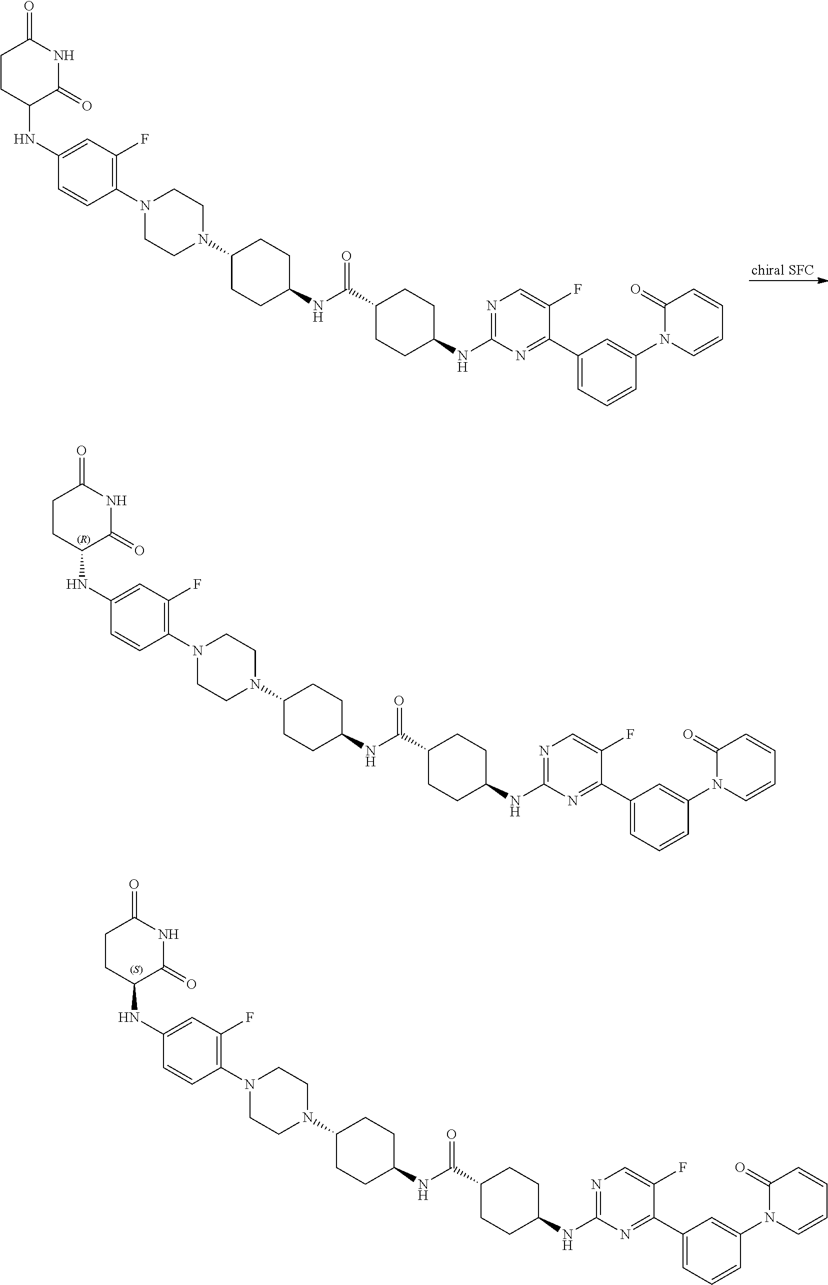

Synthesis of trans-(r,4R)—N-((1R,4R)-4-(4-(4-(((R)-2,6-dioxopiperidin-3-yl)amino)-2-fluorophenyl)piperazin-1-yl)cyclohexyl)-4-((5-fluoro-4-(3-(2-oxopyridin-1(2H)-yl)phenyl)pyrimidin-2-yl)amino)cyclohexane-1-carboxamide (Compound 420) and trans-(1r,4R)—N-((1S,4R)-4-(4-(4-(((S)-2,6-dioxopiperidin-3-yl)amino)-2-fluorophenyl)piperazin-1-yl)cyclohexyl)-4-((5-fluoro-4-(3-(2-oxopyridin-1(2H)-yl)phenyl)pyrimidin-2-yl)amino)cyclohexane-1-carboxamide (Compound 436). Trans-(1r,4R)—N-((1r,4R)-4-(4-(4-((2,6-dioxopiperidin-3-yl)amino)-2-fluorophenyl)piperazin-1-yl)cyclohexyl)-4-((5-fluoro-4-(3-(2-oxopyridin-1(2H)-yl)phenyl)pyrimidin-2-yl)amino)cyclohexane-1-carboxamide (Compound 381) (70.0 mg, 88.2 μmol, 1.00 eq) was further separated by SFC (column: REGIS(S,S)WHELK-O1 (250 mm*25 mm, 10 um); mobile phase: [CO₂-IPA (0.1% IPAm)]; B %: 60%, isocratic elution mode) to give the title compound (Compound 420) (9.70 mg, 11.3 μmol, 12.8% yield, 92.5% purity, 99.4% ee) (retention time: 2.13, peak 1) as a white solid. LCMS (ES⁺): m/z 794.4 [M+H]⁺ and give the title compound (Compound 436) (7.40 mg, 8.19 μmol, 9.29% yield, 87.9% purity, 97.0% ee) (retention time: 2.62, peak 2) as a white solid. LCMS (ES⁺): m/z 794.4 [M+H]⁺.

### Example 396. Synthesis of trans-N-(trans-4-(4-(3-(2,6-dioxopiperidin-3-yl)-1-methyl-1H-indazol-6-yl)piperazin-1-yl)cyclohexyl)-4-((5-fluoro-4-(3-(2-oxopyridin-1(2H)-yl)phenyl)pyrimidin-2-yl)amino)cyclohexane-1-carboxamide (Compound 383)

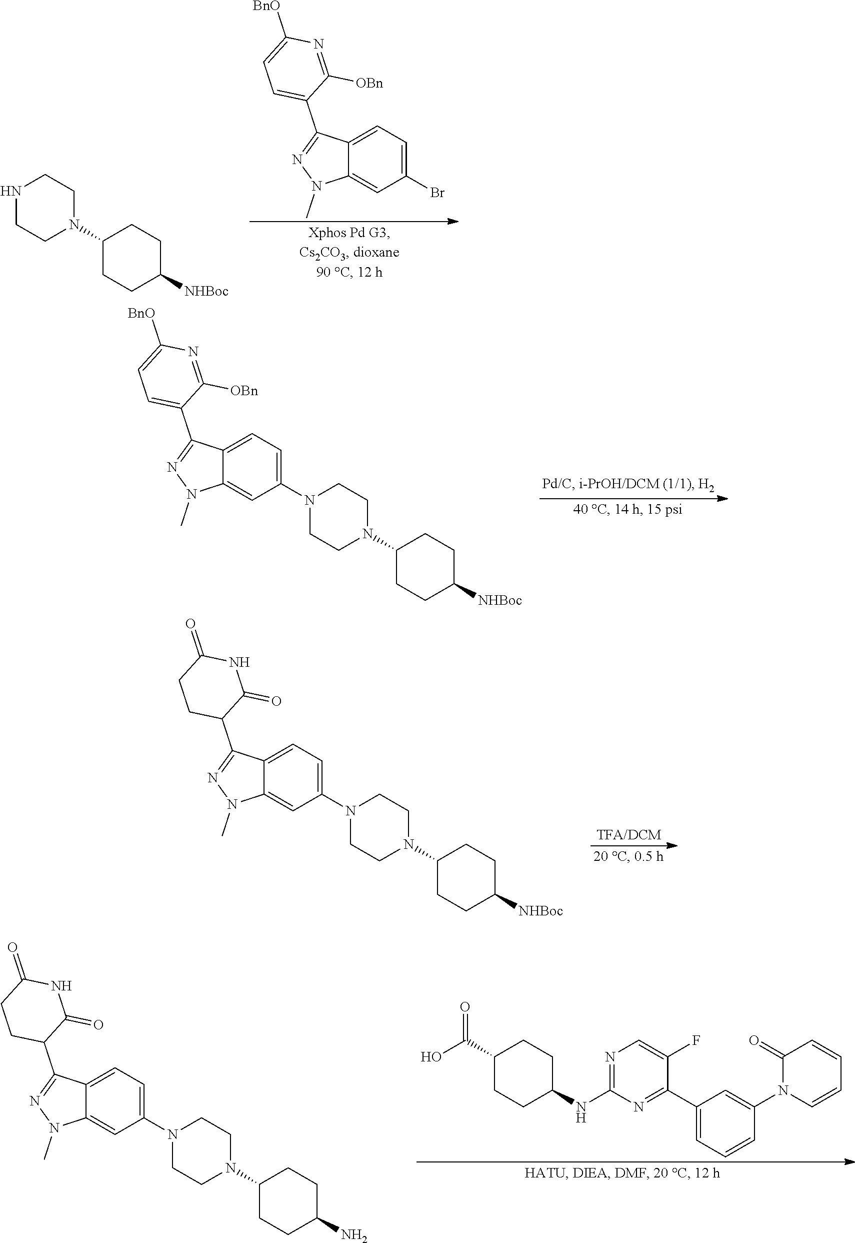

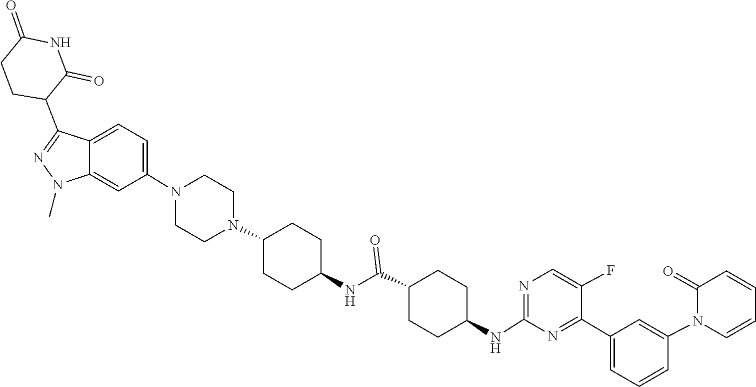 [SMILES: Cn1nc(C2CCC(=O)NC2=O)c2ccc(N3CCN([C@H]4CC[C@H](NC(=O)[C@H]5CC[C@H](Nc6ncc(F)c(-c7cccc(-n8ccccc8=O)c7)n6)CC5)CC4)CC3)cc21]

Synthesis of trans-tert-butyl (4-(4-(3-(2,6-bis(benzyloxy)pyridin-3-yl)-1-methyl-1H-indazol-6-yl)piperazin-1-yl)cyclohexyl)carbamate. To a solution of trans-tert-butyl (4-(piperazin-1-yl)cyclohexyl)carbamate (3.00 g, 10.6 mmol, 1.00 eq) and 3-(2,6-bis(benzyloxy)pyridin-3-yl)-6-bromo-1-methyl-1H-indazole (5.30 g, 10.6 mmol, 1.00 eq) in dioxane (150 mL) were added Cs₂CO₃ (6.90 g, 21.2 mmol, 2.00 eq) and Xphos Pd G3 (896 mg, 1.06 mmol, 0.100 eq) at 20 °C under N₂. The mixture was stirred at 90 °C for 12 h under N₂. LCMS showed trans-tert-butyl (4-(piperazin-1-yl)cyclohexyl)carbamate was consumed and the desired mass was detected. To the mixture was added H₂O (100 mL) and the mixture was extracted with EtOAc (100 mL*3). The combined organic layers were washed with brine (100 mL*3), dried over Na₂SO₄, filtered and the filtrate was concentrated under reduced pressure to give a residue. The residue was purified by MPLC (SiO₂, PE/EtOAc=1/0 to 0/1) to give the title compound (6.50 g, 9.25 mmol, 87.4% yield) as yellow solid. LCMS (ES⁺): m/z 703.5 [M+H]⁺.

Synthesis of trans-tert-butyl (4-(4-(3-(2,6-dioxopiperidin-3-yl)-1-methyl-1H-indazol-6-yl)piperazin-1-yl)cyclohexyl)carbamate. To a stirred solution of trans-tert-butyl (4-(4-(3-(2,6-bis(benzyloxy)pyridin-3-yl)-1-methyl-1H-indazol-6-yl)piperazin-1-yl)cyclohexyl)carbamate (3.50 g, 4.98 mmol, 1.00 eq) in DCM (20 mL) and i-PrOH (20 mL) was added 10% Pd/C (3.50 g, 50% purity) at 20 °C. The suspension was degassed and purged with H₂ for three times. The mixture was stirred under H₂ (15 psi) at 40 °C for 14 h. LCMS showed trans-tert-butyl (4-(4-(3-(2,6-bis(benzyloxy)pyridin-3-yl)-1-methyl-1H-indazol-6-yl)piperazin-1-yl)cyclohexyl)carbamate was consumed completely and the desired mass was detected. The mixture was filtered and the filtrate was concentrated under reduced pressure to give the title compound (1.80 g, crude) as a green solid. LCMS (ES⁺): m/z 525.4 [M+H]⁺.

Synthesis of trans-3-(6-(4-(4-aminocyclohexyl)piperazin-1-yl)-1-methyl-1H-indazol-3-yl)piperidine-2,6-dione. A solution of trans-tert-butyl (4-(4-(3-(2,6-dioxopiperidin-3-yl)-1-methyl-1H-indazol-6-yl)piperazin-1-yl)cyclohexyl)carbamate (900 mg, 1.72 mmol, 1.00 eq) in DCM (5 mL) and TFA (1 mL) was stirred at 20 °C for 0.5 h. LCMS showed trans-tert-butyl (4-(4-(3-(2,6-dioxopiperidin-3-yl)-1-methyl-1H-indazol-6-yl)piperazin-1-yl)cyclohexyl)carbamate was consumed and the desired mass was detected. The mixture was concentrated under reduced pressure to give the title compound (925 mg, crude, TFA) as a black oil. LCMS (ES⁺): m/z 425.3 [M+H]⁺.

Synthesis of trans-N-(trans-4-(4-(3-(2,6-dioxopiperidin-3-yl)-1-methyl-1H-indazol-6-yl)piperazin-1-yl)cyclohexyl)-4-((5-fluoro-4-(3-(2-oxopyridin-1(2H)-yl)phenyl)pyrimidin-2-yl)amino)cyclohexane-1-carboxamide (Compound 383). To a solution of trans-4-((5-fluoro-4-(3-(2-oxopyridin-1(2H)-yl)phenyl)pyrimidin-2-yl)amino)cyclohexane-1-carboxylic acid (700 mg, 1.71 mmol, 1.00 eq) and DIEA (1.33 g, 10.3 mmol, 1.8 mL, 6.00 eq) in DMF (20 mL) were added HATU (782 mg, 2.06 mmol, 1.20 eq) and trans-3-(6-(4-(4-aminocyclohexyl)piperazin-1-yl)-1-methyl-1H-indazol-3-yl)piperidine-2,6-dione (923 mg, 1.71 mmol, 1.00 eq, TFA) at 20 °C, the mixture was stirred at 20 °C for 12 h. LCMS showed trans-3-(6-(4-(4-aminocyclohexyl)piperazin-1-yl)-1-methyl-1H-indazol-3-yl)piperidine-2,6-dione was consumed and the desired mass was detected. To the mixture was added H₂O (15 mL) and the mixture was extracted with EtOAc (10 mL*3). The combined organic layers were washed with brine (10 mL*3), dried over Na₂SO₄, filtered and the filtrate was concentrated under reduced pressure to give a residue. The residue was purified by prep-HPLC (column: Phenomenex Luna C18 100*30 mm*3 um; mobile phase: [H₂O (0.2% FA)-ACN]; gradient: 10%-50% B over 8.0 min) to give the title compound (Compound 383) (20.4 mg, 23.7 μmol, 1.38% yield, 100% purity, FA) as a white solid. LCMS (ES⁺): m/z 837.5 [M+Na]⁺.

### Example 397. Synthesis of trans-3-(6-(4-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperazin-1-yl)-2-oxobenzo[d]oxazol-3 (2H)-yl)piperidine-2,6-dione (Compound 397)

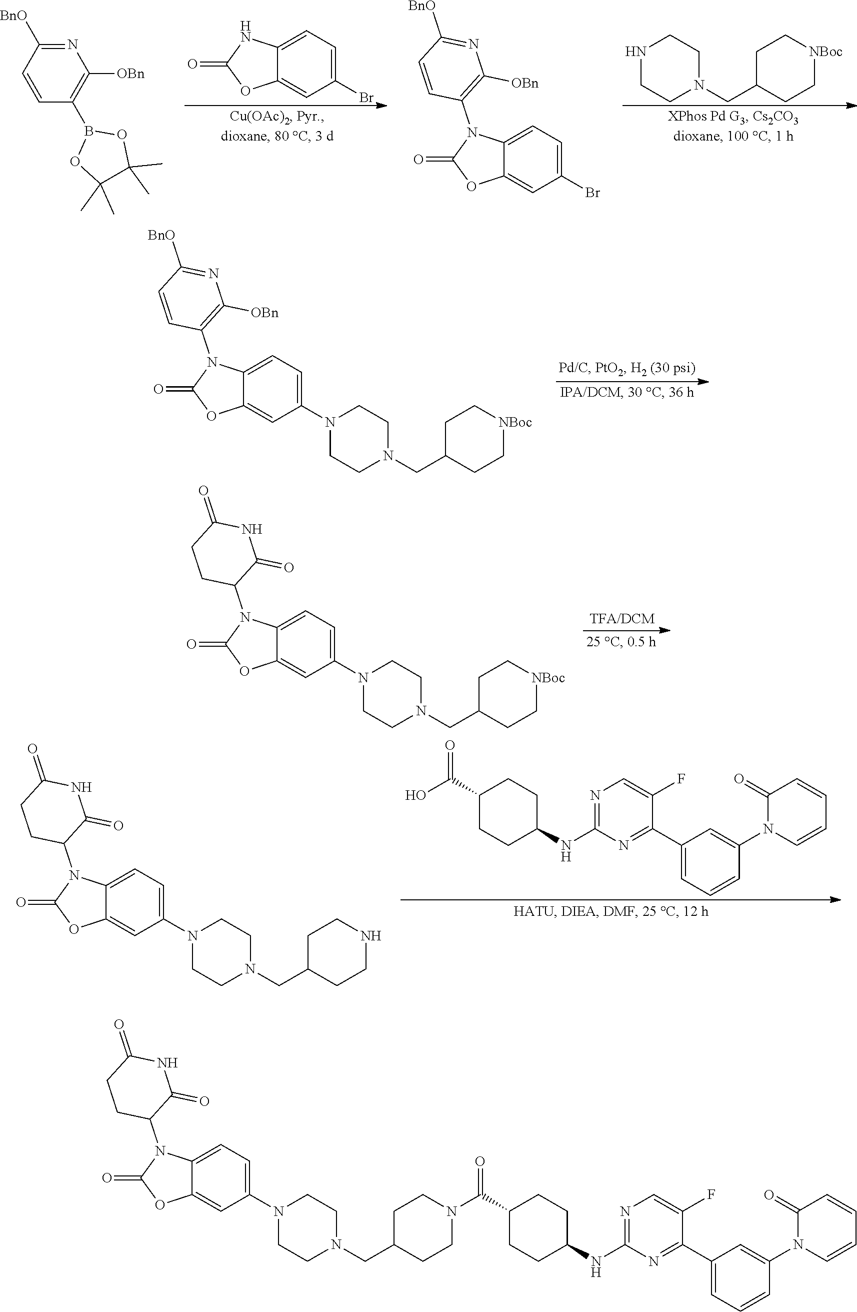

Synthesis of 3-(2,6-bis(benzyloxy)pyridin-3-yl)-6-bromobenzo[d]oxazol-2(3H)-one. To a solution of 2,6-dibenzyloxy-3-(4,4,5,5-tetramethyl-1,3,2-dioxaborolan-2-yl)pyridine (17.1 g, 41.1 mmol, 1.10 eq) in dioxane (150 mL) was added 6-bromo-3H-1,3-benzoxazol-2-one (8.00 g, 37.3 mmol, 1.00 eq), Cu(OAc)₂ (6.79 g, 37.3 mmol, 1.00 eq) and pyridine (29.5 g, 373 mmol, 30.1 mL, 10.0 eq) at 25 °C. The mixture was stirred at 80 °C for 3 days in air. LC-MS showed the desired mass was detected. The mixture was diluted with water (200 mL), and extracted with EtOAc (250 mL*3). The combined organic layers were washed with brine (200 mL), dried over Na₂SO₄, filtered and concentrated under reduced pressure. The residue was purified by column chromatography (SiO₂, Petroleum ether:Ethyl acetate=1:0 to 5:1) to give the title compound (11.6 g, 16.1 mmol, 43.1% yield, 70.0% purity) as a brown solid. LCMS (ES⁺): m/z 503.1 [M+H]⁺.

Synthesis of tert-butyl 4-((4-(3-(2,6-bis(benzyloxy)pyridin-3-yl)-2-oxo-2,3-dihydrobenzo[d]oxazol-6-yl)piperazin-1-yl)methyl)piperidine-1-carboxylate. A mixture of 3-(2,6-bis(benzyloxy)pyridin-3-yl)-6-bromobenzo[d]oxazol-2(3H)-one (4.20 g, 8.34 mmol, 1.00 eq), tert-butyl 4-(piperazin-1-ylmethyl)piperidine-1-carboxylate (2.36 g, 8.34 mmol, 1.00 eq), Cs₂CO₃ (8.16 g, 25.0 mmol, 3.00 eq), XPhos Pd G3 (706 mg, 834 μmol, 0.100 eq) in dioxane (50 mL) was degassed and purged with N₂ for 3 times at 25 °C, and then the mixture was stirred at 100 °C for 1 h under N₂ atmosphere. LC-MS showed the desired mass was detected. The mixture was diluted with water (50 mL), and extracted with EtOAc (50 mL*3). The combined organic layers were washed with brine (20 mL), dried over Na₂SO₄, filtered and concentrated under reduced pressure. The residue was purified by column chromatography (SiO₂, Petroleum ether:Ethyl acetate=1:0 to 1:1) to give the title compound (3.30 g, 4.63 mmol, 55.4% yield, 99.0% purity) as a brown solid. LCMS (ES⁺): m/z 706.5 [M+H]⁺.

Synthesis of tert-butyl 4-((4-(3-(2,6-dioxopiperidin-3-yl)-2-oxo-2,3-dihydrobenzo[d]oxazol-6-yl)piperazin-1-yl)methyl)piperidine-1-carboxylate. To a solution of tert-butyl 4-[[4-[3-(2,6-dibenzyloxy-3-pyridyl)-2-oxo-1,3-benzoxazol-6-yl]piperazin-1-yl]methyl]piperidine-1-carboxylate (3.00 g, 4.25 mmol, 1.00 eq) in IPA (15 mL) and DCM (15 mL) was added PtO₂ (1.50 g, 6.61 mmol, 1.55 eq) and 10% Pd/C (1.50 g, 7.05 mmol, 50.0% purity, 1.66 eq) under N₂ atmosphere at 25 °C. The suspension was degassed and purged with H₂ for 3 times. The mixture was stirred under H₂ (30 Psi) at 30 °C for 36 h. LC-MS showed the desired mass was detected. The reaction mixture was filtered and concentrated under reduced pressure. The residue was purified by column chromatography (SiO₂, Petroleum ether:Ethyl acetate=1:0 to 0:1) to give the title compound (900 mg, 1.36 mmol, 32.1% yield, 80.0% purity) as a light blue solid. LCMS (ES⁺): m/z 528.4 [M+H]⁺.

Synthesis of 3-(2-oxo-6-(4-(piperidin-4-ylmethyl)piperazin-1-yl)benzo[d]oxazol-3 (2H)-yl)piperidine-2,6-dione. To a solution of tert-butyl 4-((4-(3-(2,6-dioxopiperidin-3-yl)-2-oxo-2,3-dihydrobenzo[d]oxazol-6-yl)piperazin-1-yl)methyl)piperidine-1-carboxylate (200 mg, 379 μmol, 1.00 eq) in DCM (3 mL) was added TFA (43.2 mg, 379 μmol, 28.1 μL, 1.00 eq) at 25 °C. The mixture was stirred at 25 °C for 0.5 h. LC-MS showed the desired mass was detected. The reaction mixture was concentrated under reduced pressure to give the title compound (160 mg, crude, TFA) as blue oil. LCMS (ES⁺): m/z 428.2 [M+H]⁺.

Synthesis of trans-3-(6-(4-((1-4-((5-fluoro-4-(3-(2-oxopyridin-1(2H)-yl)phenyl)pyrimidin-2-yl)amino)cyclohexane-1-carbonyl)piperidin-4-yl)methyl)piperazin-1-yl)-2-oxobenzo[d]oxazol-3 (2H)-yl)piperidine-2,6-dione (Compound 397). To a solution of 3-(2-oxo-6-(4-(piperidin-4-ylmethyl)piperazin-1-yl)benzo[d]oxazol-3 (2H)-yl)piperidine-2,6-dione (160 mg, 295 μmol, 1.00 eq, TFA) in DMF (2 mL) was added DIEA (38.1 mg, 295 mol, 51.4 μL, 1.00 eq) until pH~7. To a solution of trans-4-[[5-fluoro-4-[3-(2-oxo-1-pyridyl)phenyl]pyrimidin-2-yl]amino]cyclohexanecarboxylic acid (63.1 mg, 154 μmol, 0.523 eq) in DMF (2 mL) was added DIEA (38.1 mg, 295 μmol, 51.4 μL, 1.00 eq) until pH~7. Then HATU (112 mg, 295 μmol, 1.00 eq) was added to the mixture of the two solutions. The mixture was stirred at 25 °C for 12 h. LC-MS showed the desired mass was detected. The residue was purified by prep-HPLC (column: Phenomenex Luna C18 75*30 mm*3 um; mobile phase: [H₂O (0.2% FA)-ACN]; gradient: 5%-45% B over 8.0 min) to give the title compound (Compound 397) (40.1 mg, 48.5 μmol, 16.4% yield, 98.9% purity) as a white solid. LCMS (ES⁺): m/z 818.4 [M+H]⁺.

### Example 398. Synthesis of cis-3-(6-(1-(2-(1-(3-((5-fluoro-4-(3-(2-oxopyridin-1(2H)-yl)phenyl)pyrimidin-2-yl)amino)cyclobutane-1-carbonyl)piperidin-4-yl)ethyl)piperidin-4-yl)-2-oxobenzo[d]oxazol-3 (2H)-yl)piperidine-2,6-dione (Compound 325)

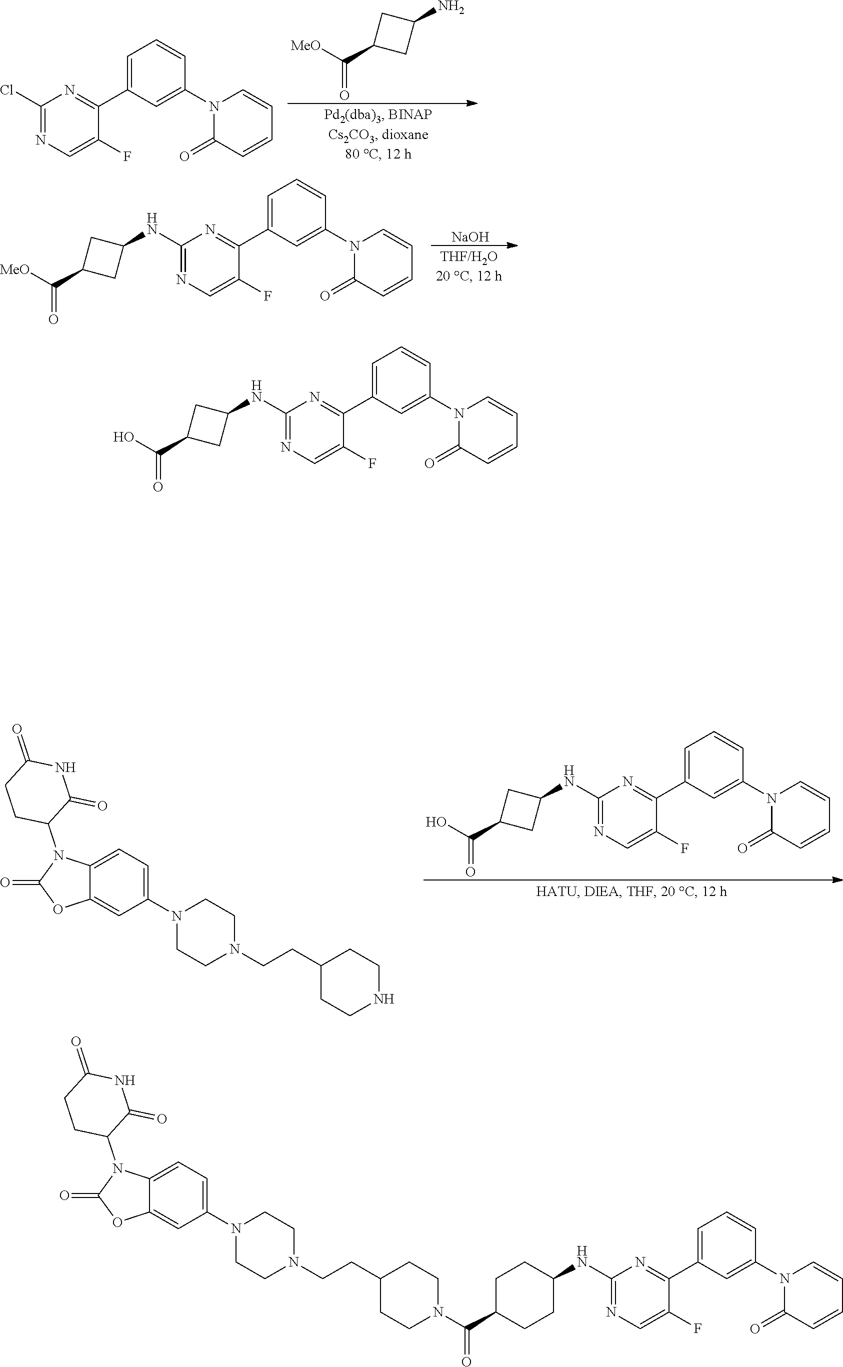

Synthesis of methyl cis-3-((5-fluoro-4-(3-(2-oxopyridin-1(2H)-yl)phenyl)pyrimidin-2-yl)amino)cyclobutane-1-carboxylate. To a solution of 1-(3-(2-chloro-5-fluoropyrimidin-4-yl)phenyl)pyridin-2(1H)-one (1.00 g, 3.31 mmol, 1.00 eq) in dioxane (15 mL) were added cis-methyl 3-aminocyclobutane-1-carboxylate (515 mg, 3.99 mmol, 1.20 eq) and Pd₂(dba)₃ (303 mg, 331 μmol, 9.98e-2 eq), BINAP (206 mg, 331 μmol, 9.98e-2 eq), Cs₂CO₃ (2.16 g, 6.63 mmol, 2.00 eq) at 20 °C. The mixture was stirred at 80 °C for 12 h under N₂. LC-MS showed that 1-(3-(2-chloro-5-fluoropyrimidin-4-yl)phenyl)pyridin-2(1H)-one was consumed completely and one main peak with the desired mass was detected. The mixture was concentrated under reduced pressure. The residue was purified by column chromatography (SiO₂, Petroleum ether:Ethyl acetate=1:0 to 0:1) to give the title compound (280 mg, 710 μmol, 21.4% yield, n/a purity) as a yellow oil. LCMS (ES⁺): m/z 395.1 [M+H]⁺.

Synthesis of cis-3-((5-fluoro-4-(3-(2-oxopyridin-1(2H)-yl)phenyl)pyrimidin-2-yl)amino)cyclobutane-1-carboxylic acid. To a solution of cis-methyl 3-((5-fluoro-4-(3-(2-oxopyridin-1(2H)-yl)phenyl)pyrimidin-2-yl)amino)cyclobutane-1-carboxylate (230 mg, 583 μmol, 1.00 eq) in THF (3.00 mL), H₂O (0.500 mL) was added NaOH (46.5 mg, 1.16 mmol, 1.99 eq) at 20 °C. The mixture was stirred at 20 °C for 12 h. LC-MS showed that cis-methyl 3-((5-fluoro-4-(3-(2-oxopyridin-1(2H)-yl)phenyl)pyrimidin-2-yl)amino)cyclobutane-1-carboxylate was consumed completely and one main peak with the desired mass was detected. 1M HCl was added to the mixture to adjust pH-4, and the mixture was filtered and the filter cake was dried in a vacuum to give the title compound (200 mg, crude, HCl) as a light yellow solid. LCMS (ES⁺): m/z 381.1 [M+H]⁺

Synthesis of cis-3-(6-(1-(2-(1-(3-((5-fluoro-4-(3-(2-oxopyridin-1(2H)-yl)phenyl)pyrimidin-2-yl)amino)cyclobutane-1-carbonyl)piperidin-4-yl)ethyl)piperidin-4-yl)-2-oxobenzo[d]oxazol-3 (2H)-yl)piperidine-2,6-dione (Compound 325). To a solution of 3-(2-oxo-6-(1-(2-(piperidin-4-yl)ethyl)piperidin-4-yl)benzo[d]oxazol-3 (2H)-yl)piperidine-2,6-dione (150 mg, 341 μmol, 1.00 eq) in THF (2.00 mL) was added DIEA (220 mg, 1.70 mmol, 297 μL, 5.00 eq) and cis-3-((5-fluoro-4-(3-(2-oxopyridin-1(2H)-yl)phenyl)pyrimidin-2-yl)amino)cyclobutane-1-carboxylic acid (130 mg, 342 μmol, 1.00 eq), HATU (388 mg, 1.02 mmol, 3.00 eq) at 20 °C. The mixture was stirred at 20 °C for 12 h. LC-MS showed that 3-(2-oxo-6-(1-(2-(piperidin-4-yl)ethyl)piperidin-4-yl)benzo[d]oxazol-3 (2H)-yl)piperidine-2,6-dione was consumed completely and one peak with the desired mass was detected. The mixture was filtered and the filtrate was concentrated under reduced pressure. The residue was purified by prep-HPLC (column: Phenomenex Luna C18 100*30 mm*3 um; mobile phase: [H₂O (0.2% FA)-ACN]; gradient: 5%-45% B over 8.0 min) to give the title compound (Compound 325) (19.3 mg, 21.5 μmol, 6.31% yield, 90.4% purity, 0.2 FA) as a yellow solid. LCMS (ES⁺): m/z 803.3 [M+H]⁺.

### Example 399. Synthesis of 3-(6-(1-((1-(2-(4-((5-fluoro-4-(3-(2-oxopyridin-1(2H)-yl)phenyl)pyrimidin-2-yl)amino)-2-oxopiperidin-1-yl)acetyl)piperidin-4-yl)methyl)piperidin-4-yl)-2-oxobenzo[d]oxazol-3 (2H)-yl)piperidine-2,6-dione (Compound 367)

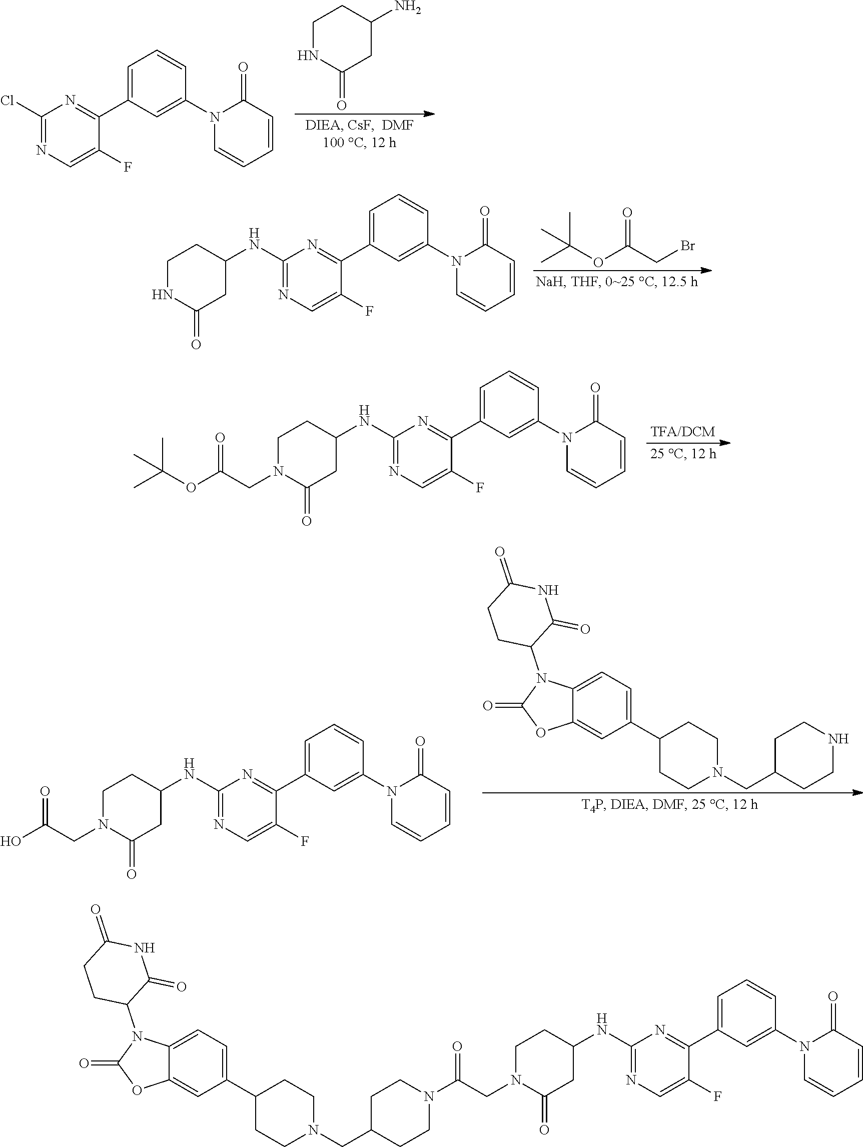

Synthesis of 1-(3-(5-fluoro-2-((2-oxopiperidin-4-yl)amino)pyrimidin-4-yl)phenyl)pyridin-2(1H)-one. To a solution of 1-[3-(2-chloro-5-fluoro-pyrimidin-4-yl)phenyl]pyridin-2-one (1.00 g, 3.31 mmol, 1.00 eq) in DMF (10 mL) was added 4-aminopiperidin-2-one (756 mg, 3.31 mmol, 1.00 eq, TFA), CsF (503 mg, 3.31 mmol, 122.35 μL, 1.00 eq) and DIEA (1.71 g, 13.2 mmol, 2.31 mL, 4.00 eq) at 25 °C. The mixture was stirred at 100 °C for 12 h. LC-MS showed desired mass was detected. The reaction mixture was diluted with H₂O 30 mL and extracted with EtOAc (30 mL*3). The combined organic layers were concentrated under reduced pressure. The residue was purified by column chromatography (SiO₂, DCM:MeOH=1:0 to 10:1) to give the title compound (340 mg, 858 μmol, 25.9% yield, 95.8% purity) as a yellow oil. LCMS (ES⁺): m/z 380.1 [M+H]⁺.

Synthesis of tert-butyl 2-(4-((5-fluoro-4-(3-(2-oxopyridin-1(2H)-yl)phenyl)pyrimidin-2-yl)amino)-2-oxopiperidin-1-yl)acetate. To a solution of 1-(3-(5-fluoro-2-((2-oxopiperidin-4-yl)amino)pyrimidin-4-yl)phenyl)pyridin-2(1H)-one (300 mg, 790 μmol, 1.00 eq) in THE (5 mL) was added NaH (37.9 mg, 948 μmol, 60.0% purity, 1.20 eq) at 0 °C. The mixture was stirred at 0 °C for 0.5 h. Then tert-butyl 2-bromoacetate (185 mg, 948 mol, 140.11 μL, 1.20 eq) was added at 0 °C. The mixture was stirred at 25 °C for 12 h. LC-MS showed desired mass was detected. The reaction mixture was quenched by addition water 10 mL at 0 °C. The residue was purified by column chromatography (SiO₂, DCM:MeOH=1:0 to 10:1) to give the title compound (200 mg, 396 μmol, 50.1% yield, 97.7% purity) as a yellow oil. LCMS (ES⁺): m/z 494.3 [M+H]⁺.

Synthesis of 2-(4-((5-fluoro-4-(3-(2-oxopyridin-1(2H)-yl)phenyl)pyrimidin-2-yl)amino)-2-oxopiperidin-1-yl)acetic acid. To a solution of tert-butyl 2-(4-((5-fluoro-4-(3-(2-oxopyridin-1(2H)-yl)phenyl)pyrimidin-2-yl)amino)-2-oxopiperidin-1-yl)acetate (200 mg, 405 mol, 1.00 eq) in DCM (2 mL) was added TFA (3.07 g, 26.9 mmol, 2 mL, 66.4 eq) at 25 °C. The mixture was stirred at 25 °C for 12 h. LC-MS showed desired mass was detected. The reaction concentrated under reduced pressure to give the title compound (200 mg, crude, TFA) as a yellow solid. LCMS (ES⁺): m/z 438.2 [M+H]⁺.

Synthesis of 3-(6-(1-((1-(2-(4-((5-fluoro-4-(3-(2-oxopyridin-1(2H)-yl)phenyl)pyrimidin-2-yl)amino)-2-oxopiperidin-1-yl)acetyl)piperidin-4-yl)methyl)piperidin-4-yl)-2-oxobenzo[d]oxazol-3 (2H)-yl)piperidine-2,6-dione (Compound 367). To a solution of 2-(4-((5-fluoro-4-(3-(2-oxopyridin-1(2H)-yl)phenyl)pyrimidin-2-yl)amino)-2-oxopiperidin-1-yl)acetic acid (100 mg, 228 μmol, 1.00 eq, TFA) in DMF (2 mL) was added 3-(2-oxo-6-(1-(piperidin-4-ylmethyl)piperidin-4-yl)benzo[d]oxazol-3 (2H)-yl)piperidine-2,6-dione (97.5 mg, 180 μmol, 0.789 eq, TFA), DIEA (88.6 mg, 685 μmol, 119 μL, 3.00 eq) and T₄P (247 mg, 342 μmol, 50.0% purity, 1.50 eq) at 25 °C. The mixture was stirred at 25 °C for 1 h. LC-MS showed desired mass was detected. The reaction mixture was concentrated under reduced pressure. The residue was purified by prep-HPLC (column: 3_Phenomenex Luna C18 75*30 mm*3 um; mobile phase: [H₂O (0.1% TFA)-ACN]; gradient: 5%-35% B over 9.0 min). (column: Waters Xbridge BEH C18 100*30 mm*10 um; mobile phase: [H₂O (10 mM NH₄HCO₃)-ACN]; gradient: 50%-80% B over 8.0 min). (column: Phenomenex Luna C18 100*30 mm*3 um; mobile phase: [H₂O (0.2% FA)-ACN]; gradient: 1%-40% B over 8.0 min) to give the title compound (Compound 367) (4.70 mg, 5.27 μmol, 2.30% yield, 100% purity, FA) as a white solid. LCMS (ES⁺): m/z 846.4 [M+H]⁺.

### Example 400. Synthesis of 3-(5-(1-(2-(1-((1R,3S)-3-((4-([1,1′-biphenyl]-3-yl)-5-chloropyrimidin-2-yl)amino)cyclohexane-1-carbonyl)piperidin-4-yl)ethyl)piperidin-4-yl)-3-methyl-2-oxo-2,3-dihydro-1H-benzo[d]imidazol-1-yl)piperidine-2,6-dione (Compound 280)

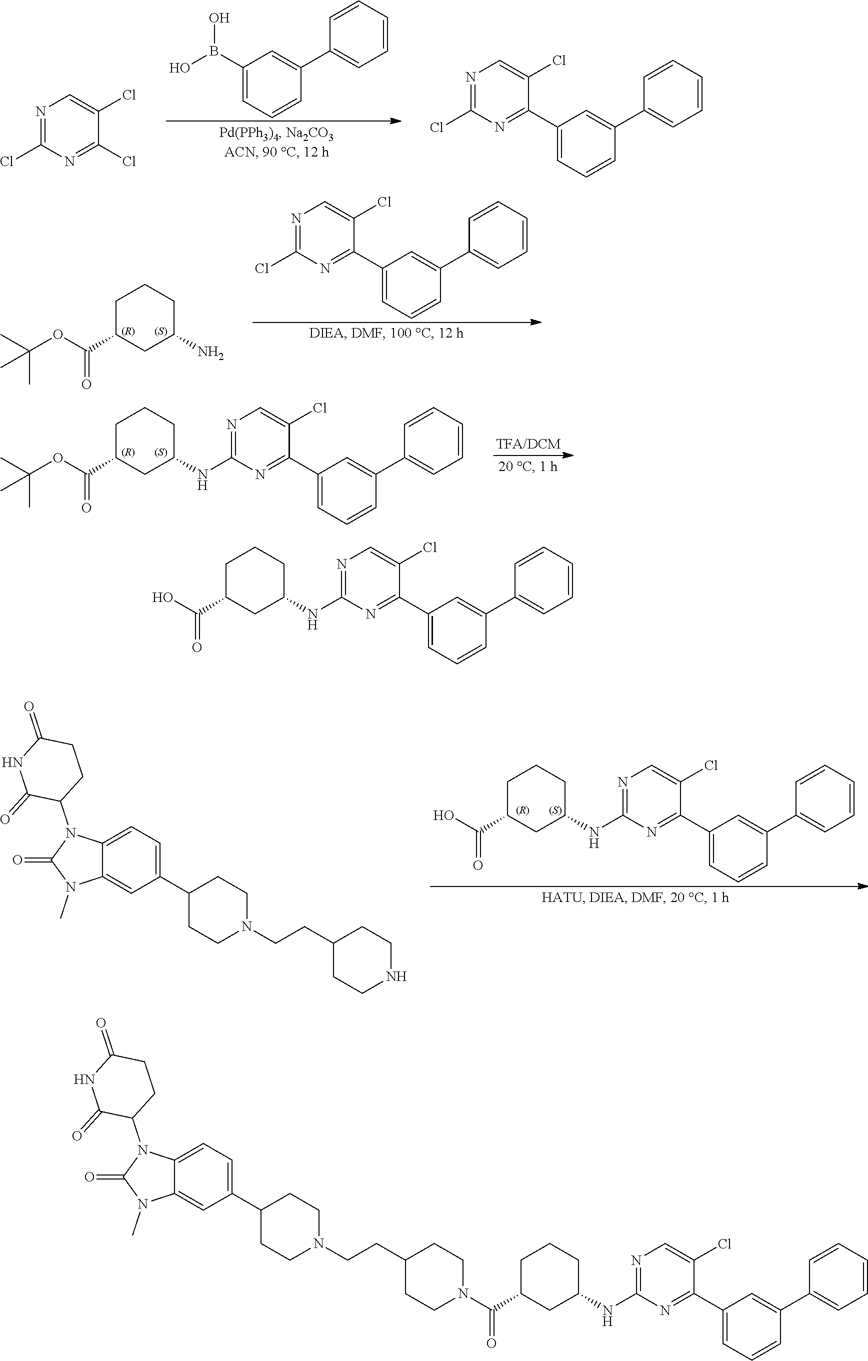

Synthesis of 4-([1,1′-biphenyl]-3-yl)-2,5-dichloropyrimidine. To a solution of 2,4,5-trichoropyrimidine (23.16 g, 126.25 mmol, 1 eq), (3-phenylphenyl)boronic acid (25 g, 126.25 mmol, 1 eq), Pd(PPh₃)₄ (1.46 g, 1.26 mmol, 0.01 eq) and Na₂CO₃ (26.76 g, 252.49 mmol, 2 eq) in CH₃CN (750 mL) was degassed and purged with N₂ for 3 times, and then the mixture was stirred at 90 °C for 12 h under N₂ atmosphere. LCMS showed the desired mass was detected. The reaction mixture was filtered and the filtrate was concentrated under reduced pressure to give a residue. The residue was triturated with MeOH (1 L) at 25 °C for 1 h, then filtered and filter cake was dried under reduced pressure to give the title compound (75 g, 242.03 mmol, 47.93% yield, 97.190% purity) as a white solid. LCMS (ES⁺): m/z 300.9 [M+H]⁺.

Synthesis of tert-butyl (1R,3S)-3-((4-([1,1′-biphenyl]-3-yl)-5-chloropyrimidin-2-yl)amino)cyclohexane-1-carboxylate. To a solution of tert-butyl (1R,3S)-3-aminocyclohexane-1-carboxylate (600 mg, 3.01 mmol, 1.00 eq) in DMF (20 mL) were added DIEA (1.17 g, 9.03 mmol, 1.57 mL, 3.00 eq) and 2,5-dichloro-4-(3-phenylphenyl)pyrimidine (907 mg, 3.01 mmol, 1.00 eq) at 20 °C. The mixture was stirred at 100 °C for 12 h. LCMS showed tert-butyl (1R,3S)-3-aminocyclohexane-1-carboxylate was consumed completely and the desired mass was detected. Water (20 mL) was added to the mixture at 25 °C. Then the reaction mixture was extracted with EtOAc (15 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=2:1 to 1:1) to give the title compound (1.00 g, 2.16 mmol, 71.6% yield) as a yellow oil. LCMS (ES⁺): m/z 464.2 [M+H]⁺.

Synthesis of (1R,3S)-3-((4-([1,1′-biphenyl]-3-yl)-5-chloropyrimidin-2-yl)amino)cyclohexane-1-carboxylic acid. To a solution of tert-butyl (1R,3S)-3-((4-([1,1′-biphenyl]-3-yl)-5-chloropyrimidin-2-yl)amino)cyclohexane-1-carboxylate (300 mg, 647 μmol, 1.00 eq) in DCM (6 mL) was added TFA (2 mL) at 20 °C. The mixture was stirred at 20 °C for 1 h. LCMS showed tert-butyl (1R,3S)-3-((4-([1,1′-biphenyl]-3-yl)-5-chloropyrimidin-2-yl)amino)cyclohexane-1-carboxylate was consumed completely and the desired mass was detected. The reaction mixture was concentrated under reduced pressure to give the title compound (260 mg, crude) as a yellow oil. LCMS (ES⁺): m/z 408.2 [M+H]⁺.

Synthesis of 3-(5-(1-(2-(1-((1R,3S)-3-((4-([1,1′-biphenyl]-3-yl)-5-chloropyrimidin-2-yl)amino)cyclohexane-1-carbonyl)piperidin-4-yl)ethyl)piperidin-4-yl)-3-methyl-2-oxo-2,3-dihydro-1H-benzo[d]imidazol-1-yl)piperidine-2,6-dione (Compound 280). To a solution of 3-(3-methyl-2-oxo-5-(1-(2-(piperidin-4-yl)ethyl)piperidin-4-yl)-2,3-dihydro-1H-benzo[d]imidazol-1-yl)piperidine-2,6-dione (300 mg, 529 μmol, 1.00 eq, TFA) in DMF (3 mL) were added HATU (301 mg, 793 μmol, 1.50 eq), DIEA (342 mg, 2.64 mmol, 460 μL, 5.00 eq) and (1R,3S)-3-((4-([1,1′-biphenyl]-3-yl)-5-chloropyrimidin-2-yl)amino)cyclohexane-1-carboxylic acid (216 mg, 529 μmol, 1.00 eq) at 20 °C. The mixture was stirred at 20 °C for 1 h. LCMS showed 3-(3-methyl-2-oxo-5-(1-(2-(piperidin-4-yl)ethyl)piperidin-4-yl)-2,3-dihydro-1H-benzo[d]imidazol-1-yl)piperidine-2,6-dione was consumed completely and the desired mass was detected. The reaction was filtered and the filtrate was concentrated under reduced pressure to give a residue. The residue was purified by prep-HPLC (column: Phenomenex Luna C18 100*30 mm*3 um; mobile phase: [1H₂O (0.2% FA)-ACN]; gradient: 25%-65% B over 8.0 min) to give the title compound (Compound 280) (34.0 mg, 37.6 μmol, 7.11% yield, 98.4% purity, FA) as a white solid. LCMS (ES⁺): m/z 843.4 [M+H]⁺

### Example 401. Synthesis of 3-(5-(1-(2-(1-((1S,3R)-3-((4-([1,1′-biphenyl]-3-yl)-5-chloropyrimidin-2-yl)amino)cyclohexane-1-carbonyl)piperidin-4-yl)ethyl)piperidin-4-yl)-3-methyl-2-oxo-2,3-dihydro-1H-benzo[d]imidazol-1-yl)piperidine-2,6-dione (Compound 281)

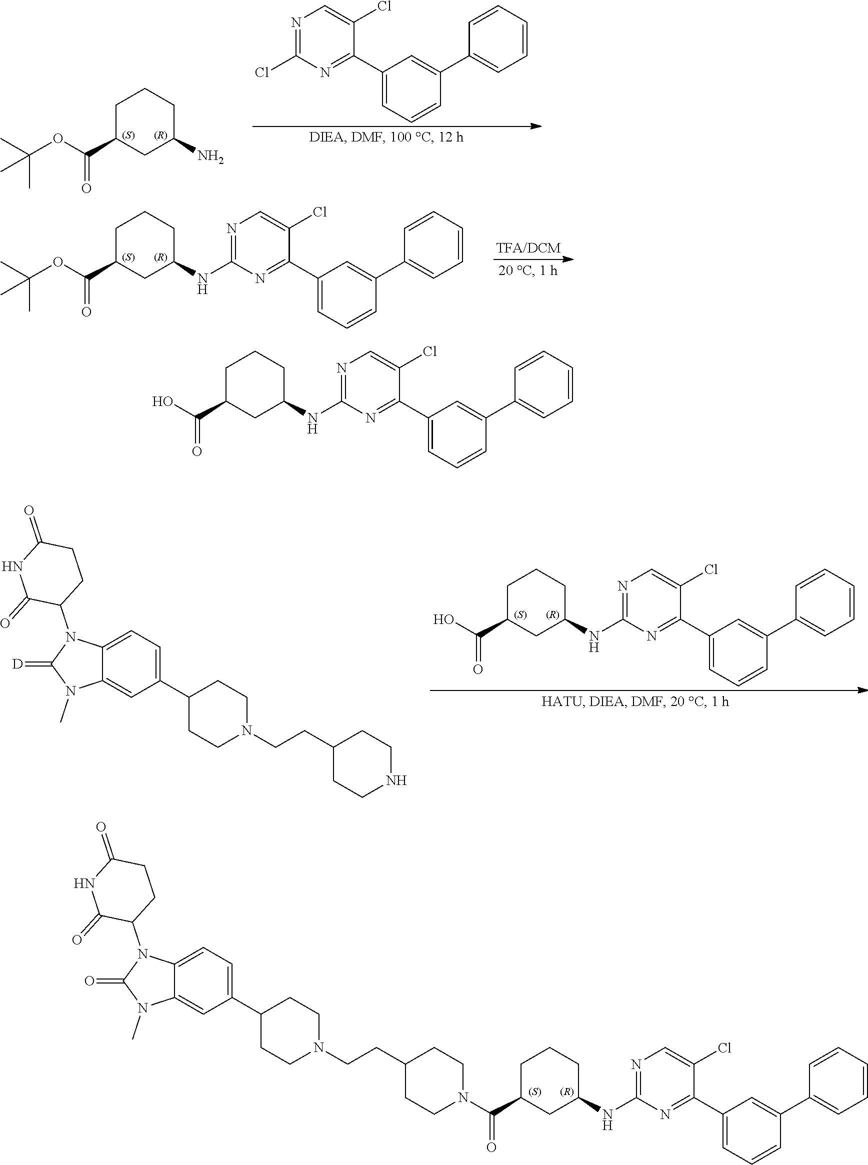

Synthesis of tert-butyl (1S,3R)-3-((4-([1,1′-biphenyl]-3-yl)-5-chloropyrimidin-2-yl)amino)cyclohexane-1-carboxylate. To a solution of tert-butyl (1S,3R)-3-aminocyclohexane-1-carboxylate (580 mg, 2.91 mmol, 1.00 eq) in DMF (20 mL) were added DIEA (1.13 g, 8.73 mmol, 1.52 mL, 3.00 eq) and 2,5-dichloro-4-(3-phenylphenyl)pyrimidine (877 mg, 2.91 mmol, 1.00 eq) at 20 °C. The mixture was stirred at 100 °C for 12 h. LCMS showed tert-butyl (1S,3R)-3-aminocyclohexane-1-carboxylate was consumed completely and the desired mass was detected. Water (20 mL) was added to the mixture at 25 °C. Then the reaction mixture was extracted with EtOAc (15 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=2:1 to 1:1) to give the title compound (1.00 g, 2.16 mmol, 74.1% yield) as a yellow oil. LCMS (ES⁺): m/z 464.2 [M+H]⁺.

Synthesis of (1S,3R)-3-((4-([1,1′-biphenyl]-3-yl)-5-chloropyrimidin-2-yl)amino)cyclohexane-1-carboxylic acid. To a solution of tert-butyl (1S,3R)-3-((4-([1,1′-biphenyl]-3-yl)-5-chloropyrimidin-2-yl)amino)cyclohexane-1-carboxylate (300 mg, 647 mol, 1.00 eq) in DCM (6 mL) was added TFA (2 mL) at 20 °C. The mixture was stirred at 20 °C for 1 h. LCMS showed tert-butyl (1S,3R)-3-((4-([1,1′-biphenyl]-3-yl)-5-chloropyrimidin-2-yl)amino)cyclohexane-1-carboxylate was consumed completely and the desired mass was detected. The reaction mixture was concentrated under reduced pressure to give the title compound (260 mg, crude) as a yellow oil. LCMS (ES⁺): m/z 408.2 [M+H]⁺.

Synthesis of 3-(5-(1-(2-(1-((1S,3R)-3-((4-([1,1′-biphenyl]-3-yl)-5-chloropyrimidin-2-yl)amino)cyclohexane-1-carbonyl)piperidin-4-yl)ethyl)piperidin-4-yl)-3-methyl-2-oxo-2,3-dihydro-1H-benzo[d]imidazol-1-yl)piperidine-2,6-dione (Compound 281). To a solution of 3-(3-methyl-2-oxo-5-(1-(2-(piperidin-4-yl)ethyl)piperidin-4-yl)-2,3-dihydro-1H-benzo[d]imidazol-1-yl)piperidine-2,6-dione (300 mg, 529 μmol, 1.00 eq, TFA) in DMF (3 mL) were added HATU (301 mg, 793 μmol, 1.50 eq), DIEA (342 mg, 2.64 mmol, 460 μL, 5.00 eq) and (1S,3R)-3-((4-([1,1′-biphenyl]-3-yl)-5-chloropyrimidin-2-yl)amino)cyclohexane-1-carboxylic acid (216 mg, 529 μmol, 1.00 eq) at 20 °C. The mixture was stirred at 20 °C for 1 h. LCMS showed 3-(3-methyl-2-oxo-5-(1-(2-(piperidin-4-yl)ethyl)piperidin-4-yl)-2,3-dihydro-1H-benzo[d]imidazol-1-yl)piperidine-2,6-dione was consumed completely and the desired mass was detected. The reaction mixture was filtered and the filtrate was concentrated under reduced pressure to give a residue. The residue was purified by prep-HPLC (column: Phenomenex Luna C18 100*30 mm*3 um; mobile phase: [H₂O (0.2% FA)-ACN]; gradient: 25%-65% B over 8.0 min) to give the title compound (Compound 281) (86.6 mg, 96.8 μmol, 18.3% yield, 99.4% purity, FA) as a white solid. LCMS (ES⁺): m/z 843.4 [M+H]⁺

### Example 402. Synthesis of 3-(5-(1-((1-(2-(4-((4-([1,1′-biphenyl]-3-yl)-5-fluoropyrimidin-2-yl)amino)piperidin-1-yl)-2-oxoethyl)piperidin-4-yl)methyl)piperidin-4-yl)-3-methyl-2-oxo-2,3-dihydro-1H-benzo[d]imidazol-1-yl)piperidine-2,6-dione (Compound 286)

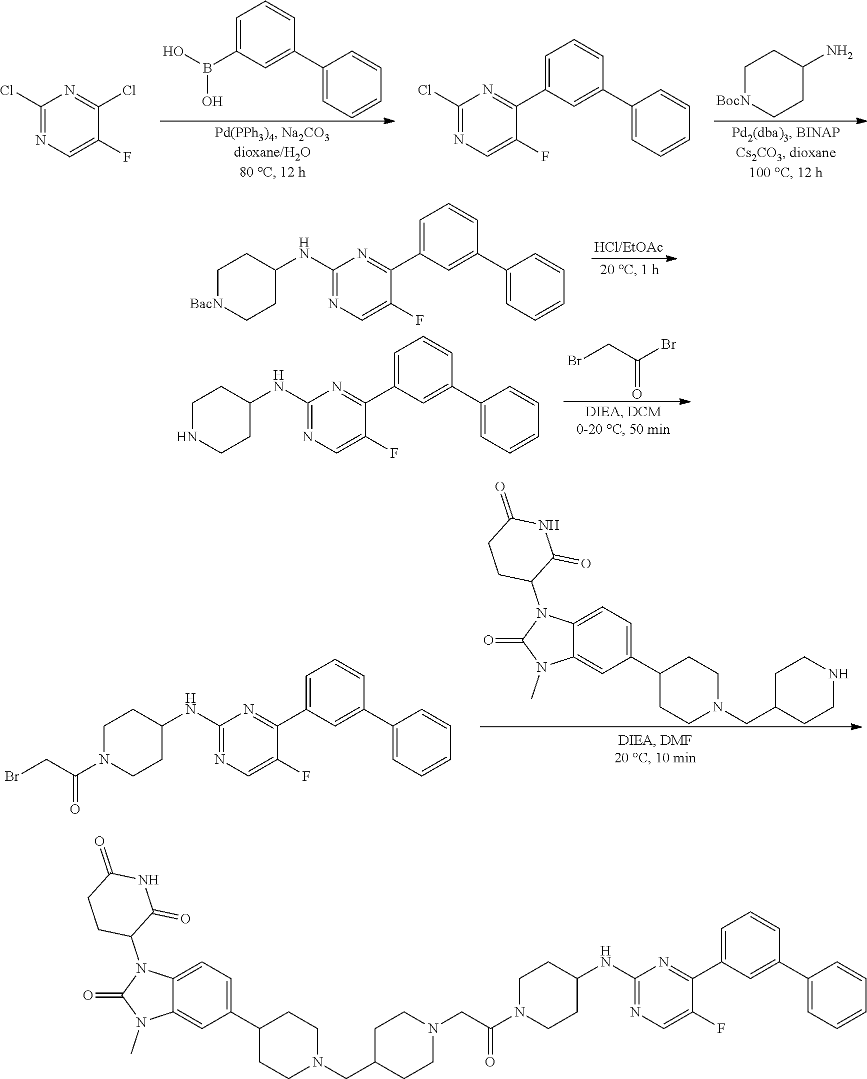

Synthesis of 4-([1,1′-biphenyl]-3-yl)-2-chloro-5-fluoropyrimidine. To a solution of 2,4-dichloro-5-fluoropyrimidine (3.38 g, 20.2 mmol, 1.00 eq) and [1,1′-biphenyl]-3-ylboronic acid (4.00 g, 20.2 mmol, 1.00 eq) in dioxane (70 mL) and H₂O (7 mL) were added Na₂CO₃ (6.42 g, 60.6 mmol, 3.00 eq) and Pd(PPh₃)₄ (1.17 g, 1.01 mmol, 0.0500 eq) at 20 °C under N₂, the mixture was stirred at 80 °C for 12 h under N₂ atmosphere. LCMS showed the desired target MS was detected. H₂O (80 mL) was added to the solution, the mixture was extracted with EtOAc (80 mL*3), the combined organic layers were dried over Na₂SO₄, filtered and the filtrate was concentrated under reduced pressure to give a residue. The residue was purified by MPLC (SiO₂, Petroleum ether/Ethyl acetate=1/0 to 1/1) to give the title compound (2.00 g, 7.02 mmol, 34.8% yield) as a yellow oil. LCMS (ES⁺): m/z 285.0 [M+H]⁺

Synthesis of tert-butyl 4-((4-([1,1′-biphenyl]-3-yl)-5-fluoropyrimidin-2-yl)amino)piperidine-1-carboxylate. To a solution of 4-([1,1′-biphenyl]-3-yl)-2-chloro-5-fluoropyrimidine (1.90 g, 6.67 mmol, 1.00 eq) and tert-butyl 4-aminopiperidine-1-carboxylate (1.34 g, 6.67 mmol, 1.00 eq) in dioxane (50 mL) were added BINAP (249 mg, 400 μmol, 0.0600 eq), Pd₂(dba)₃ (244 mg, 267 μmol, 0.0400 eq) and Cs₂CO₃ (4.35 g, 13.4 mmol, 2.00 eq) at 20 °C under N₂, the mixture was stirred at 100 °C for 12 h under N₂ atmosphere. LCMS showed 4-([1,1′-biphenyl]-3-yl)-2-chloro-5-fluoropyrimidine was consumed and the desired target MS was detected. H₂O (50 mL) was added to the solution, then the mixture was extracted with EtOAc (30 mL*3), the combined organic layers were dried over Na₂SO₄, filtered and the filtrate was concentrated under reduced pressure to give a residue. The residue was purified by MPLC (SiO₂, Petroleum ether/Ethyl acetate=1/0 to 1/1) to give the title compound (1.50 g, 3.34 mmol, 50.1% yield) as a yellow solid. LCMS (ES⁺): m/z 449.2 [M+H]⁺

Synthesis of 4-([1,1′-biphenyl]-3-yl)-5-fluoro-N-(piperidin-4-yl)pyrimidin-2-amine. A solution of tert-butyl 4-((4-([1,1′-biphenyl]-3-yl)-5-fluoropyrimidin-2-yl)amino)piperidine-1-carboxylate (190 mg, 424 μmol, 1.00 eq) in HCl/EtOAc (4 M, 2 mL) was stirred at 20 °C for 1 h. LCMS showed tert-butyl 4-((4-([1,1′-biphenyl]-3-yl)-5-fluoropyrimidin-2-yl)amino)piperidine-1-carboxylate was consumed and the desired target MS was detected. The mixture was concentrated under reduced pressure to give the title compound (160 mg, crude) as a white solid. LCMS (ES⁺): m/z 349.1 [M+H]⁺

Synthesis of 1-(4-((4-([1,1′-biphenyl]-3-yl)-5-fluoropyrimidin-2-yl)amino)piperidin-1-yl)-2-bromoethan-1-one. To a solution of 4-([1,1′-biphenyl]-3-yl)-5-fluoro-N-(piperidin-4-yl)pyrimidin-2-amine. (100 mg, 287 μmol, 1.00 eq) in DCM (2 mL) were added 2-bromoacetyl bromide (69.5 mg, 344 μmol, 30.0 μL, 1.20 eq) and DIEA (111 mg, 861 μmol, 150 μL, 3.00 eq) at 0 °C. The mixture was stirred at 20 °C for 20 min. LCMS showed 4-([1,1′-biphenyl]-3-yl)-5-fluoro-N-(piperidin-4-yl)pyrimidin-2-amine remained and the desired target MS was detected. Then the mixture was stirred at 20 °C for another 30 min. LCMS showed 4-([1,1′-biphenyl]-3-yl)-5-fluoro-N-(piperidin-4-yl)pyrimidin-2-amine was consumed and the desired target MS was detected. The mixture was concentrated under reduced pressure to give the title compound (120 mg, crude) as a brown liquid. LCMS (ES⁺): m/z 469.1 [M+H]⁺

Synthesis of 3-(5-(1-((1-(2-(4-((4-([1,1′-biphenyl]-3-yl)-5-fluoropyrimidin-2-yl)amino)piperidin-1-yl)-2-oxoethyl)piperidin-4-yl)methyl)piperidin-4-yl)-3-methyl-2-oxo-2,3-dihydro-1H-benzo[d]imidazol-1-yl)piperidine-2,6-dione (Compound 286). To a solution of 3-(3-methyl-2-oxo-5-(1-(piperidin-4-ylmethyl)piperidin-4-yl)-2,3-dihydro-1H-benzo[d]imidazol-1-yl)piperidine-2,6-dione (122 mg, 256 μmol, 1.00 eq, HCl) in DMF (2 mL) was added DIEA at 20 °C to adjust pH to 8. Then to the mixture was added 1-(4-((4-([1,1′-biphenyl]-3-yl)-5-fluoropyrimidin-2-yl)amino)piperidin-1-yl)-2-bromoethan-1-one (120 mg, 256 μmol, 1.00 eq) at 20 °C, the mixture was stirred at 20 °C for 10 min. LCMS showed 1-(4-((4-([1,1′-biphenyl]-3-yl)-5-fluoropyrimidin-2-yl)amino)piperidin-1-yl)-2-bromoethan-1-one was consumed and the desired target MS was detected. The mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 10%-40% B over 8.0 min) to give the title compound (Compound 286) (26.4 mg, 30.9 μmol, 40.3% yield, 97.0% purity) as a white solid. LCMS (ES⁺): m/z 828.4 [M+H]⁺

### Example 403. Synthesis of 3-(6-(1-((1-(2-(4-((4-([1,1′-biphenyl]-3-yl)-5-fluoropyrimidin-2-yl)amino)-2-oxopiperidin-1-yl)acetyl)piperidin-4-yl)methyl)piperidin-4-yl)-2-oxobenzo[d]oxazol-3 (2H)-yl)piperidine-2,6-dione (Compound 366)

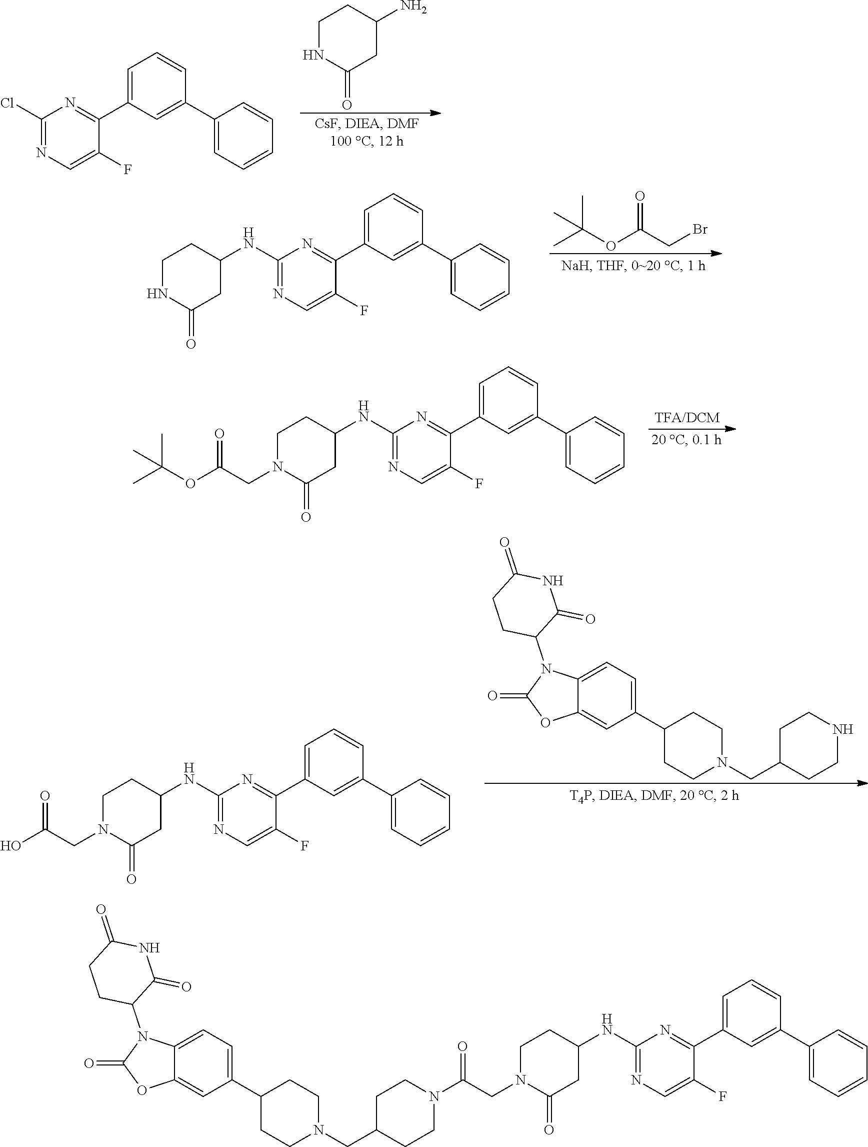

Synthesis of 4-((4-([1,1′-biphenyl]-3-yl)-5-fluoropyrimidin-2-yl)amino)piperidin-2-one. To a solution of 4-([1,1′-biphenyl]-3-yl)-2-chloro-5-fluoropyrimidine (3.00 g, 10.5 mmol, 1.00 eq) and 4-aminopiperidin-2-one (1.44 g, 12.6 mmol, 1.20 eq) in DMF (30 mL) was added DIEA (2.72 g, 21.0 mmol, 3.67 mL, 2.00 eq) and CsF (3.20 g, 21.0 mmol, 777 μL, 2.00 eq) at 20 °C. The mixture was stirred at 100 °C for 12 h. LC-MS showed the desired mass was detected. The reaction mixture was diluted with H₂O 30 mL and extracted with EtOAc 30 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (2.40 g, 3.70 mmol, 17.5% yield, 55.8% purity) as a yellow solid. LCMS (ES⁺): m/z 363.2 [M+H]⁺.

Synthesis of tert-butyl 2-(4-((4-([1,1′-biphenyl]-3-yl)-5-fluoropyrimidin-2-yl)amino)-2-oxopiperidin-1-yl)acetate. To a solution of 4-((4-([1,1′-biphenyl]-3-yl)-5-fluoropyrimidin-2-yl)amino)piperidin-2-one (600 mg, 1.66 mmol, 1.00 eq) in THF (10 mL) was added dropwise NaH (79.4 mg, 1.99 mmol, 60% purity, 1.20 eq) at 0 °C. After addition, the mixture was stirred at this temperature for 0.5 h and then tert-butyl 2-bromoacetate (387 mg, 1.99 mmol, 293 μL, 1.20 eq) was added dropwise at 0 °C. The resulting mixture was stirred at 20 °C for 0.5 h. LC-MS showed the desired mass was detected. The reaction mixture was quenched by addition H₂O 10 mL at 0 °C, and then extracted with EtOAc 10 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (100 mg, 111 μmol, 6.72% yield, 53% purity) as a yellow solid. LCMS (ES⁺): m/z 477.1 [M+H]⁺.

Synthesis of 2-(4-((4-([1,1′-biphenyl]-3-yl)-5-fluoropyrimidin-2-yl)amino)-2-oxopiperidin-1-yl)acetic acid. To a solution of tert-butyl 2-(4-((4-([1,1′-biphenyl]-3-yl)-5-fluoropyrimidin-2-yl)amino)-2-oxopiperidin-1-yl)acetate (100 mg, 209 μmol, 1.00 eq) in DCM (1 mL) was added TFA (460 mg, 4.04 mmol, 0.300 mL, 19.2 eq) at 20 °C. The mixture was stirred at 20 °C for 0.1 h. LC-MS showed the desired mass was detected. The reaction mixture was concentrated under reduced pressure to give the title compound (90.0 mg, crude) as a black solid. LCMS (ES⁺): m/z 421.1 [M+H]⁺.

Synthesis of 3-(6-(1-((1-(2-(4-((4-([1,1′-biphenyl]-3-yl)-5-fluoropyrimidin-2-yl)amino)-2-oxopiperidin-1-yl)acetyl)piperidin-4-yl)methyl)piperidin-4-yl)-2-oxobenzo[d]oxazol-3 (2H)-yl)piperidine-2,6-dione (Compound 366). To a solution of 2-(4-((4-([1,1′-biphenyl]-3-yl)-5-fluoropyrimidin-2-yl)amino)-2-oxopiperidin-1-yl)acetic acid (70.0 mg, 166 μmol, 1.00 eq) and 3-(2-oxo-6-(1-(piperidin-4-ylmethyl)piperidin-4-yl)benzo[d]oxazol-3 (2H)-yl)piperidine-2,6-dione (71.0 mg, 166 μmol, 1.00 eq) in DMF (2 mL) was added DIEA (43.0 mg, 332 μmol, 58.0 μL, 2.00 eq) and T₄P (119 mg, 332 μmol, 2.00 eq) at 20 °C. The mixture was stirred at 20 °C for 2 h. LC-MS showed the desired mass was detected. The reaction mixture was concentrated under reduced pressure. The residue was purified by prep-HPLC (column: Waters Xbridge BEH C18 100*30 mm*10 um; mobile phase: [H₂O (10 mM NH₄HCO₃)-ACN]; gradient: 40%-70% B over 8.0 min) to give the title compound (Compound 366) (4.50 mg, 5.40 μmol, 3.24% yield, 99.4% purity) as a light yellow solid. LCMS (ES⁺): m/z 829.4 [M+H]⁺.

### Example 404. Synthesis of 3-(5-(1-((1-(2-(4-((5-fluoro-4-(4′-fluoro-[1,1′-biphenyl]-3-yl)pyrimidin-2-yl)amino)piperidin-1-yl)-2-oxoethyl)piperidin-4-yl)methyl)piperidin-4-yl)-3-methyl-2-oxo-2,3-dihydro-1H-benzo[d]imidazol-1-yl)piperidine-2,6-dione (Compound 288)

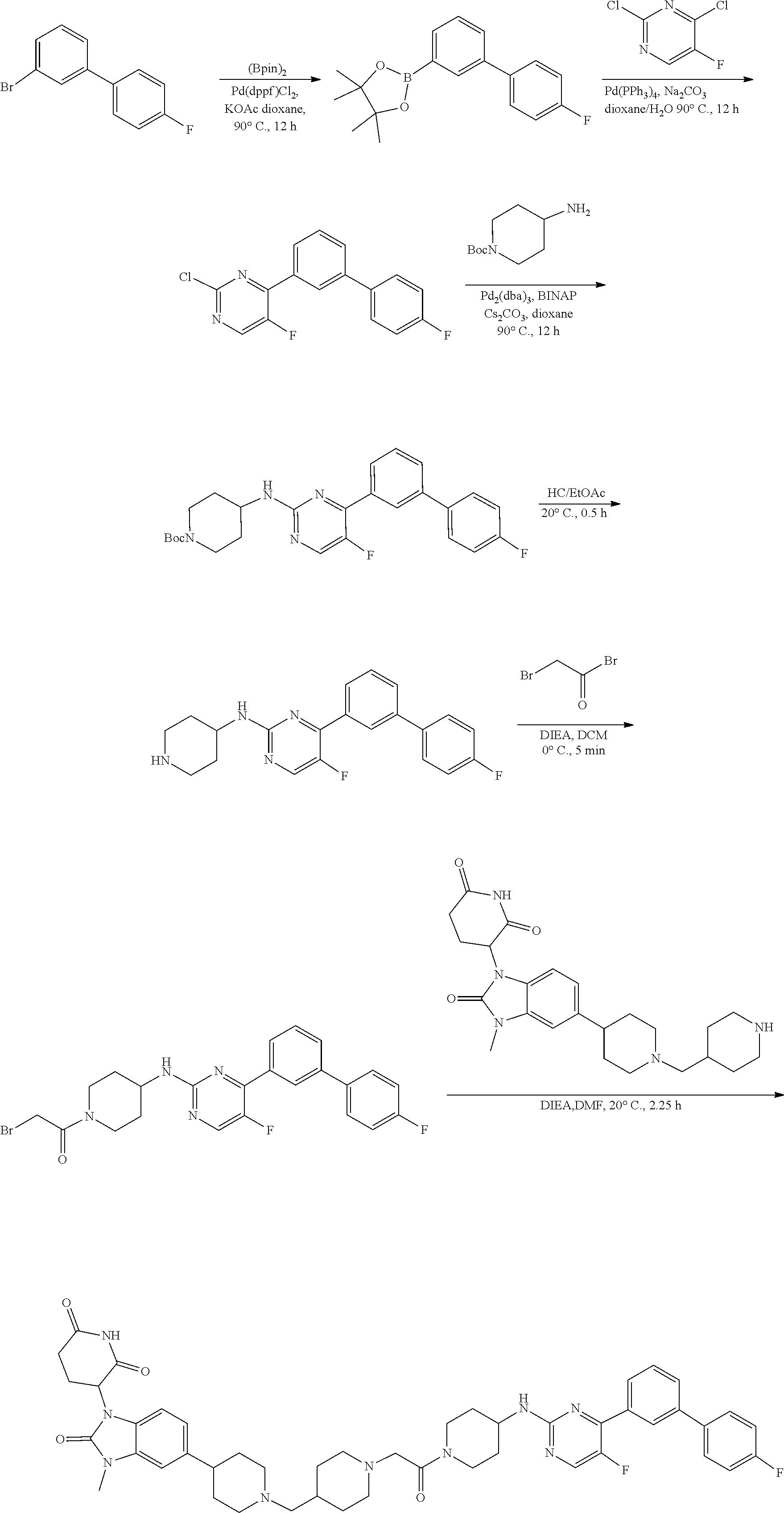

Synthesis of 2-(4′-fluoro-[1,1′-biphenyl]-3-yl)-4,4,5,5-tetramethyl-1,3,2-dioxaborolane. To a solution of 3-bromo-4′-fluoro-1,1′-biphenyl (5.00 g, 19.9 mmol, 1.00 eq) and 4,4,4′,4′,5,5,5′,5′-octamethyl-2,2′-bi(1,3,2-dioxaborolane) (6.07 g, 23.9 mmol, 1.20 eq) in dioxane (50 mL) were added KOAc (3.91 g, 39.8 mmol, 2.00 eq) and Pd(dppf)Cl₂ (729 mg, 996 μmol, 0.0500 eq) at 20 °C under N₂, the mixture was stirred at 90 °C for 12 h under N₂. TLC (SiO₂, PE/EtOAc=10/1) showed 3-bromo-4′-fluoro-1,1′-biphenyl was consumed completely and two new spots formed. To the mixture was added H₂O (20 mL) and the mixture was extracted with EtOAc (20 mL*3). The combined organic layers were washed with brine (20 mL*3), dried over Na₂SO₄, filtered and the filtrate was concentrated under reduced pressure to give a residue. The residue was purified by MPLC (SiO₂, PE/EtOAc=1/0 to 10/1) to give the title compound (5.30 g, 17.8 mmol, 89.3% yield) as a yellow solid. The product couldn't be detected by LCMS. It was confirmed by ¹H NMR (400 MHz, DMSO-d₆) δ=7.91 (s, 1H), 7.80 (br d, J=7.8 Hz, 1H), 7.75-7.66 (m, 3H), 7.58-7.47 (m, 1H), 7.32 (t, J=8.8 Hz, 2H), 1.34 (s, 12H)

Synthesis of 2-chloro-5-fluoro-4-(4′-fluoro-[1,1′-biphenyl]-3-yl)pyrimidine. To a solution of 2-(4′-fluoro-[1,1′-biphenyl]-3-yl)-4,4,5,5-tetramethyl-1,3,2-dioxaborolane (1.00 g, 3.35 mmol, 1.00 eq) and 2,4-dichloro-5-fluoropyrimidine (1.12 g, 6.71 mmol, 2.00 eq) in dioxane (20 mL) and H₂O (2 mL) were added Na₂CO₃ (711 mg, 6.71 mmol, 2.00 eq) and Pd(PPh₃)₄ (77.5 mg, 67.1 μmol, 0.0200 eq) at 20 °C under N₂. The mixture was stirred at 90 °C for 12 h under N₂ atmosphere. LCMS showed the desired mass was detected. To the mixture was added H₂O (20 mL) and the mixture was extracted with EtOAc (20 mL*3). The combined organic layers were washed with brine (20 mL*3), dried over Na₂SO₄, filtered and the filtrate was concentrated under reduced pressure to give a residue. The residue was purified by MPLC (SiO₂, PE/EtOAc=1/0 to 5/1) to give the title compound (810 mg, 2.68 mmol, 79.8% yield) as a yellow solid. LCMS (ES⁺): m/z 303.1 [M+H]⁺

Synthesis of tert-butyl 4-((5-fluoro-4-(4′-fluoro-[1,1′-biphenyl]-3-yl)pyrimidin-2-yl)amino)piperidine-1-carboxylate. To a solution of 2-chloro-5-fluoro-4-(4′-fluoro-[1,1′-biphenyl]-3-yl)pyrimidine (780 mg, 2.58 mmol, 1.00 eq) and tert-butyl 4-aminopiperidine-1-carboxylate (516 mg, 2.58 mmol, 1.00 eq) in dioxane (5 mL) were added Cs₂CO₃ (1.68 g, 5.15 mmol, 2.00 eq), BINAP (96.3 mg, 155 μmol, 0.0600 eq) and Pd₂(dba)₃ (94.4 mg, 103 mol, 0.0400 eq) at 20 °C under N₂. The resulting mixture was stirred at 90 °C for 12 h under N₂. LCMS showed 2-chloro-5-fluoro-4-(4′-fluoro-[1,1′-biphenyl]-3-yl)pyrimidine was consumed and the desired mass was detected. To the mixture was added H₂O (10 mL) and the mixture was extracted with EtOAc (10 mL*3). The combined organic layers were washed with brine (10 mL*3), dried over Na₂SO₄, filtered and the filtrate was concentrated under reduced pressure to give a residue. The residue was purified by prep-TLC (SiO₂, PE/EtOAc=1/1) to give the title compound (900 mg, 1.93 mmol, 74.9% yield) as a yellow solid. LCMS (ES⁺): m/z 467.2 [M+H]⁺

Synthesis of 5-fluoro-4-(4′-fluoro-[1,1′-biphenyl]-3-yl)-N-(piperidin-4-yl)pyrimidin-2-amine. A solution of tert-butyl 4-((5-fluoro-4-(4′-fluoro-[1,1′-biphenyl]-3-yl)pyrimidin-2-yl)amino)piperidine-1-carboxylate (380 mg, 815 μmol, 1.00 eq) in HCl/EtOAc (3 mL) was stirred at 20 °C for 0.5 h. LCMS showed tert-butyl 4-((5-fluoro-4-(4′-fluoro-[1,1′-biphenyl]-3-yl)pyrimidin-2-yl)amino)piperidine-1-carboxylate was consumed and the desired mass was detected. The mixture was concentrated under reduced pressure to give the title compound (330 mg, crude, HCl) as a white solid. LCMS (ES⁺): m/z 367.1 [M+H]⁺.

Synthesis of 2-bromo-1-(4-((5-fluoro-4-(4′-fluoro-[1,1′-biphenyl]-3-yl)pyrimidin-2-yl)amino)piperidin-1-yl)ethan-1-one. To a solution of 5-fluoro-4-(4′-fluoro-[1,1′-biphenyl]-3-yl)-N-(piperidin-4-yl)pyrimidin-2-amine (70.0 mg, 174 μmol, 1.00 eq, HCl) in DCM (2 mL) was added DIEA (67.4 mg, 521 μmol, 90.8 μL, 3.00 eq) and 2-bromoacetyl bromide (52.6 mg, 261 μmol, 22.7 μL, 1.50 eq) at 0 °C. The mixture was stirred at 0 °C for 5 min. LCMS showed 5-fluoro-4-(4′-fluoro-[1,1′-biphenyl]-3-yl)-N-(piperidin-4-yl)pyrimidin-2-amine was consumed and the desired mass was detected. The mixture was concentrated under reduced pressure to give the title compound (84.0 mg, crude) as a brown oil. LCMS (ES⁺): m/z 487.2 [M+H]⁺.

Synthesis of 3-(5-(1-((1-(2-(4-((5-fluoro-4-(4′-fluoro-[1,1′-biphenyl]-3-yl)pyrimidin-2-yl)amino)piperidin-1-yl)-2-oxoethyl)piperidin-4-yl)methyl)piperidin-4-yl)-3-methyl-2-oxo-2,3-dihydro-1H-benzo[d]imidazol-1-yl)piperidine-2,6-dione (Compound 288). To a solution of 3-(3-methyl-2-oxo-5-(1-(piperidin-4-ylmethyl)piperidin-4-yl)-2,3-dihydro-1H-benzo[d]imidazol-1-yl)piperidine-2,6-dione (80.0 mg, 168 μmol, 1.00 eq, HCl) in DMF (2 mL) was added DIEA (65.2 mg, 504 μmol, 87.8 μL, 3.00 eq) and 2-bromo-1-(4-((5-fluoro-4-(4′-fluoro-[1,1′-biphenyl]-3-yl)pyrimidin-2-yl)amino)piperidin-1-yl)ethan-1-one (81.9 mg, 168 μmol, 1.00 eq) at 0 °C. The mixture was stirred at 20 °C for 15 min. LCMS showed 2-bromo-1-(4-((5-fluoro-4-(4′-fluoro-[1,1′-biphenyl]-3-yl)pyrimidin-2-yl)amino)piperidin-1-yl)ethan-1-one remained and the desired mass was not detected. Then the mixture was stirred at 20 °C for another 2 h. LCMS showed 2-bromo-1-(4-((5-fluoro-4-(4′-fluoro-[1,1′-biphenyl]-3-yl)pyrimidin-2-yl)amino)piperidin-1-yl)ethan-1-one was consumed completely and the desired mass was detected. The mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 5%-45% B over 8.0 min) to give the title compound (Compound 288) (19.0 mg, 22.3 μmol, 13.3% yield, 99.2% purity) as a yellow solid. LCMS (ES⁺): m/z 846.3 [M+H]⁺

### Example 405. Synthesis of trans-N-(4-((4-(1-(cyclopropanecarbonyl)piperidin-3-yl)-5-fluoropyrimidin-2-yl)amino)cyclohexyl)-2-(4-((4-(3-(2,6-dioxopiperidin-3-yl)-2-oxo-2,3-dihydrobenzo[d]oxazol-6-yl)piperidin-1-yl)methyl)piperidin-1-yl)acetamide (Compound 359)

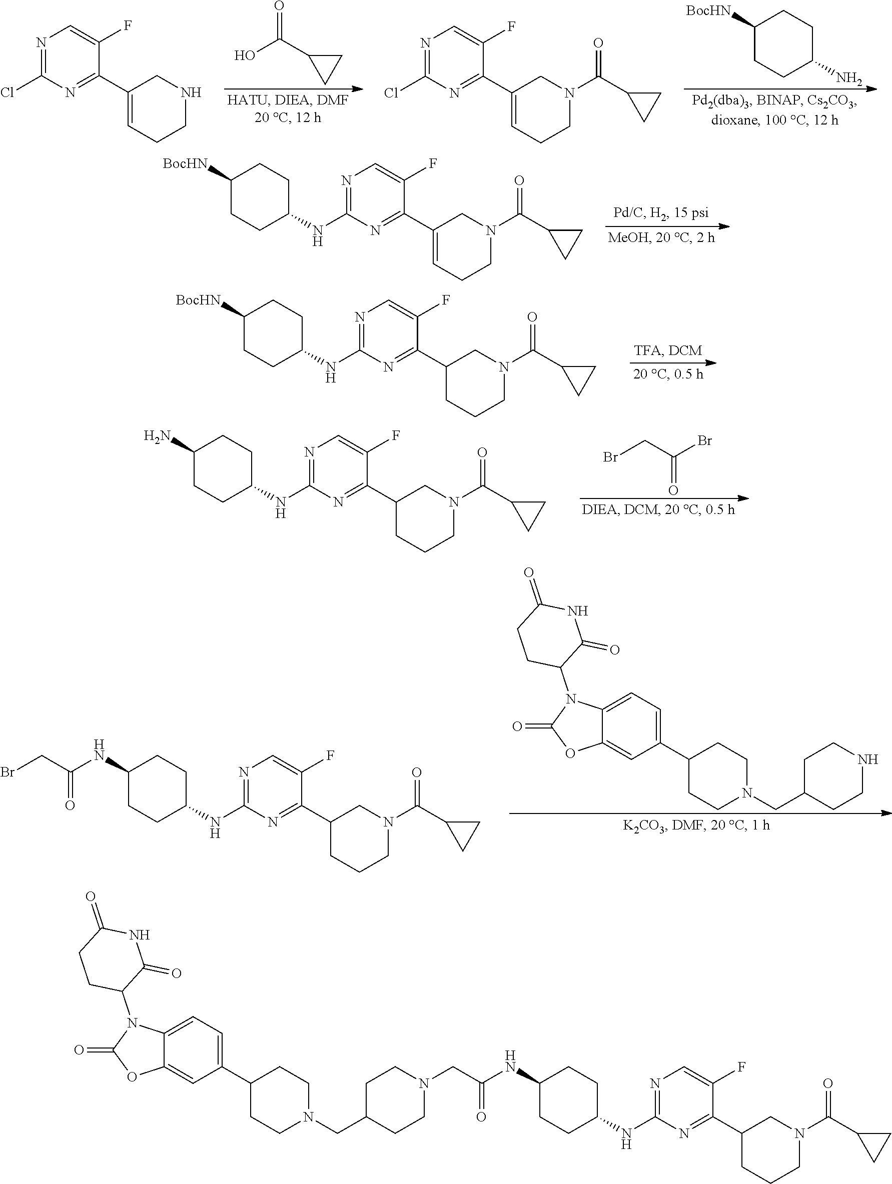

Synthesis of [5-(2-chloro-5-fluoro-pyrimidin-4-yl)-3,6-dihydro-2H-pyridin-1-yl]-cyclopropyl-methanone. To a solution of 2-chloro-5-fluoro-4-(1,2,3,6-tetrahydropyridin-5-yl)pyrimidine (800 mg, 3.20 mmol, 1.00 eq, HCl) in DMF (1 mL) was added HATU (1.82 g, 4.80 mmol, 1.50 eq) and DIEA (826 mg, 6.40 mmol, 1.11 mL, 2.00 eq) and cyclopropanecarboxylic acid (413 mg, 4.80 mmol, 379 μL, 1.50 eq) at 20 °C. The mixture was stirred at 20 °C for 12 h. LC-MS showed the desired mass was detected. The reaction mixture was quenched by addition H₂O 30 mL, and then extracted with EtOAc (100 mL*3). The combined organic layers were dried over Na₂SO₄, filtered and concentrated under reduced pressure. The residue was purified by column chromatography (SiO₂, Petroleum ether:Ethyl acetate=1:0 to 3:1) to give the title compound (900 mg, crude) as a yellow oil. LCMS (ES⁺): m/z 282.2[M+H]⁺.

Synthesis of trans-tert-butyl (4-((4-(1-(cyclopropanecarbonyl)-1,2,5,6-tetrahydropyridin-3-yl)-5-fluoropyrimidin-2-yl)amino)cyclohexyl)carbamate. A mixture of [5-(2-chloro-5-fluoro-pyrimidin-4-yl)-3,6-dihydro-2H-pyridin-1-yl]-cyclopropyl-methanone (500 mg, 1.77 mmol, 1.00 eq), trans-tert-butyl (4-aminocyclohexyl)carbamate (760 mg, 3.55 mmol, 2.00 eq), Cs₂CO₃ (1.73 g, 5.32 mmol, 3.00 eq), BINAP (110 mg, 177 μmol, 0.10 eq) and Pd₂(dba)₃ (162 mg, 177 μmol, 0.10 eq) in dioxane (5 mL) was degassed and purged with N₂ at 20 °C for 3 times, and then the mixture was stirred at 100 °C for 12 h under N₂ atmosphere. LC-MS showed the desired mass was detected. The reaction mixture was quenched by addition H₂O 5 mL, and then extracted with EtOAc (5 mL*3). The combined organic layers were dried over Na₂SO₄, filtered and concentrated under reduced pressure t. The residue was purified by prep-TLC (SiO₂, Petroleum ether:Ethyl acetate=1:2) to give the title compound (200 mg, 435 μmol, 24.5% yield) as a yellow oil. LCMS (ES⁺): m/z 460.3 [M+H]⁺.

Synthesis of trans-tert-butyl (4-((4-(1-(cyclopropanecarbonyl)piperidin-3-yl)-5-fluoropyrimidin-2-yl)amino)cyclohexyl)carbamate. A mixture of trans-tert-butyl (4-((4-(1-(cyclopropanecarbonyl)-1,2,5,6-tetrahydropyridin-3-yl)-5-fluoropyrimidin-2-yl)amino)cyclohexyl)carbamate (340 mg, 739 μmol, 1.00 eq), 10% Pd/C (157 mg, 739 μmol, 50% purity, 1.00 eq) in MeOH (2 mL) at 20 °C under N₂ atmosphere. The suspension was degassed and purged with H₂ for 3 times. The mixture was stirred under H₂ (15 Psi) at 20 °C for 2 h. LC-MS showed the desired mass was detected. The reaction mixture was filtered and the filtrate concentrated under high vacuum to give the title compound (340 mg, crude) as a yellow oil. LCMS (ES⁺): m/z 462.3 [M+H]⁺.

Synthesis of trans-(3-(2-((4-aminocyclohexyl)amino)-5-fluoropyrimidin-4-yl)piperidin-1-yl)(cyclopropyl)methanone. To a solution of trans-tert-butyl (4-((4-(1-(cyclopropanecarbonyl)piperidin-3-yl)-5-fluoropyrimidin-2-yl)amino)cyclohexyl)carbamate (150 mg, 324 μmol, 1.00 eq) in DCM (1 mL) was added TFA (1.54 g, 13.46 mmol, 1 mL, 41.4 eq) at 20 °C. The mixture was stirred at 20 °C for 0.5 h. LC-MS showed the desired mass was detected. The reaction mixture was concentrated under reduced pressure to give the title compound (150 mg, crude, TFA) as a yellow oil. LCMS (ES⁺): m/z 362.2[M+H]⁺.

Synthesis of trans-2-bromo-N-(4-((4-(1-(cyclopropanecarbonyl)piperidin-3-yl)-5-fluoropyrimidin-2-yl)amino)cyclohexyl)acetamide. To a solution of trans-(3-(2-((4-aminocyclohexyl)amino)-5-fluoropyrimidin-4-yl)piperidin-1-yl)(cyclopropyl)methanone (100 mg, 210 μmol, 1.00 eq, TFA) in DCM (1 mL) was added 2-bromoacetyl bromide (42.4 mg, 210 μmol, 18.3 μL, 1.00 eq) and DIEA (81.5 mg, 630 μmol, 109 μL, 3.00 eq) at 20 °C. The mixture was stirred at 20 °C for 0.5 h. LC-MS showed the desired mass was detected. The reaction mixture was concentrated under high vacuum to give the title compound (100 mg, crude) as a yellow oil. LCMS (ES⁺): m/z 482.2 [M+H]⁺.

Synthesis of trans-N-(4-((4-(1-(cyclopropanecarbonyl)piperidin-3-yl)-5-fluoropyrimidin-2-yl)amino)cyclohexyl)-2-(4-((4-(3-(2,6-dioxopiperidin-3-yl)-2-oxo-2,3-dihydrobenzo[d]oxazol-6-yl)piperidin-1-yl)methyl)piperidin-1-yl)acetamide (Compound 359). To a solution of trans-2-bromo-N-(4-((4-(1-(cyclopropanecarbonyl)piperidin-3-yl)-5-fluoropyrimidin-2-yl)amino)cyclohexyl)acetamide (90.0 mg, 186 μmol, 1.00 eq) in DMF (1 mL) was added K₂CO₃ (51.5 mg, 373 μmol, 2.00 eq) and 3-(2-oxo-6-(1-(piperidin-4-ylmethyl)piperidin-4-yl)benzo[d]oxazol-3 (2H)-yl)piperidine-2,6-dione (100 mg, 186 μmol, 1.00 eq, TFA) at 20 °C. The mixture was stirred at 20 °C for 1 h. LC-MS showed the desired mass was detected. The reaction mixture was quenched by addition H₂O 10 mL, and then extracted with EtOAc (10 mL*3). The combined organic layers were dried over Na₂SO₄, filtered and concentrated under reduced pressure to give a residue. The residue was purified by prep-HPLC (column: Phenomenex luna C18 100*40 mm*3 um; mobile phase: [H₂O (0.2% FA)-ACN]; gradient: 1%-35% B over 8.0 min]; gradient: 20%-50% B over 8.0 min) to give the title compound (Compound 359) (9.90 mg, 10.9 μmol, 5.89% yield, 95.4% purity, 0.70 FA) as a white solid. LCMS (ES⁺): m/z 828.5[M+H]⁺.

### Example 406. Synthesis of trans-N-(4-((5-chloro-4-(3-(2-oxopyridin-1(2H)-yl)phenyl)pyrimidin-2-yl)amino)cyclohexyl)-2-(4-((4-(3-(2,6-dioxopiperidin-3-yl)-2-oxo-2,3-dihydrobenzo[d]oxazol-6-yl)piperidin-1-yl)methyl)piperidin-1-yl)acetamide (Compound 369)

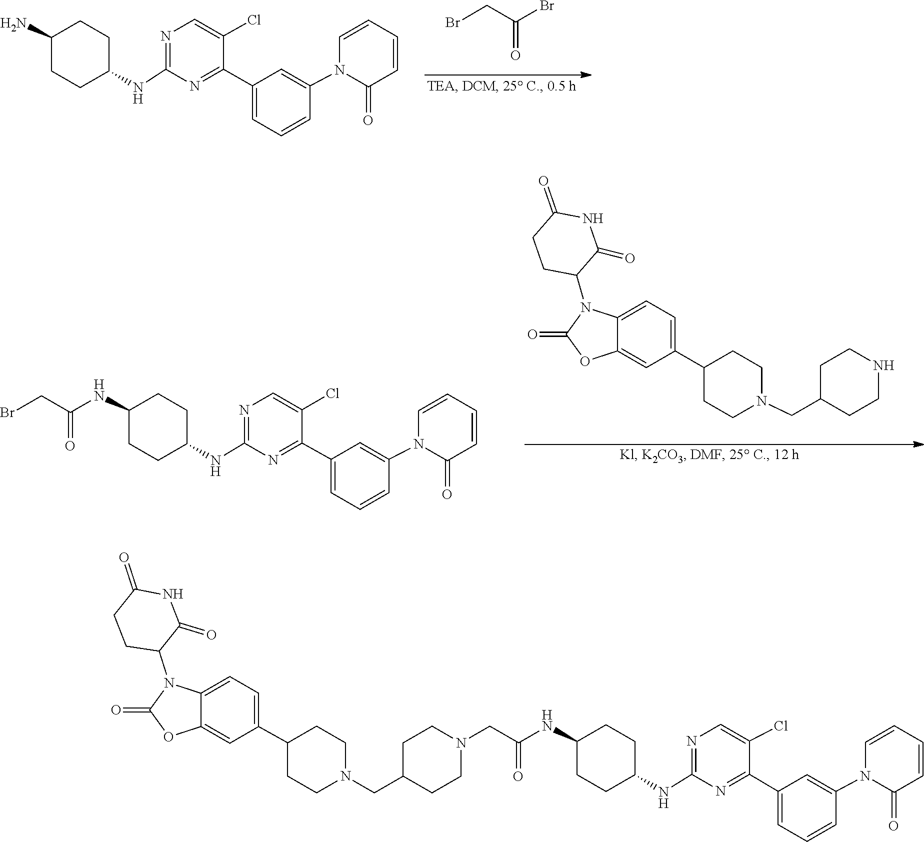 [SMILES: N[C@H]1CC[C@H](Nc2ncc(Cl)c(-c3cccc(-n4ccccc4=O)c3)n2)CC1 ; O=C(Br)CBr ; O=C(CBr)N[C@H]1CC[C@H](Nc2ncc(Cl)c(-c3cccc(-n4ccccc4=O)c3)n2)CC1 ; O=C1CCC(n2c(=O)oc3cc(C4CCN(CC5CCNCC5)CC4)ccc32)C(=O)N1 ; O=C1CCC(n2c(=O)oc3cc(C4CCN(CC5CCN(CC(=O)N[C@H]6CC[C@H](Nc7ncc(Cl)c(-c8cccc(-n9ccccc9=O)c8)n7)CC6)CC5)CC4)ccc32)C(=O)N1]

Synthesis of trans-2-bromo-N-(4-((5-chloro-4-(3-(2-oxopyridin-1(2H)-yl)phenyl)pyrimidin-2-yl)amino)cyclohexyl)acetamide. To a solution of trans-1-(3-(2-((4-aminocyclohexyl)amino)-5-chloropyrimidin-4-yl)phenyl)pyridin-2(1H)-one (150 mg, 294 μmol, 1.00 eq, TFA) in DCM (3 mL) was added TEA (119 mg, 1.18 mmol, 163 μL, 4.00 eq) and 2-bromoacetyl bromide (59.3 mg, 294 μmol, 25.6 μL, 1.00 eq) at 25 °C. The mixture was stirred at 25 °C for 0.5 h. LC-MS showed trans-1-(3-(2-((4-aminocyclohexyl)amino)-5-chloropyrimidin-4-yl)phenyl)pyridin-2(1H)-one was consumed completely and the desired mass was detected. The reaction was concentrated under reduced pressure. The residue was purified by prep-TLC (SiO₂, Petroleum ether:Ethyl acetate=1:2) to give the title compound (80.0 mg, 137 μmol, 46.7% yield, 88.9% purity) a yellow solid. LCMS (ES⁺): m/z 516.2, 518.2 [M+H]⁺.

Synthesis of trans-N-(4-((5-chloro-4-(3-(2-oxopyridin-1(2H)-yl)phenyl)pyrimidin-2-yl)amino)cyclohexyl)-2-(4-((4-(3-(2,6-dioxopiperidin-3-yl)-2-oxo-2,3-dihydrobenzo[d]oxazol-6-yl)piperidin-1-yl)methyl)piperidin-1-yl)acetamide (Compound 369). To a solution of trans-2-bromo-N-(4-((5-chloro-4-(3-(2-oxopyridin-1(2H)-yl)phenyl)pyrimidin-2-yl)amino)cyclohexyl)acetamide (60.0 mg, 116 μmol, 1.00 eq) in DMF (5 mL) was added 3-(2-oxo-6-(1-(piperidin-4-ylmethyl)piperidin-4-yl)benzo[d]oxazol-3 (2H)-yl)piperidine-2,6-dione (49.5 mg, 91.6 μmol, 7.89 eq, TFA), KI (19.2 mg, 116 μmol, 1.00 eq) and K₂CO₃ (48.1 mg, 348 μmol, 3.00 eq) at 25 °C. The mixture was stirred at 25 °C for 12 h. LC-MS showed trans-2-bromo-N-(4-((5-chloro-4-(3-(2-oxopyridin-1(2H)-yl)phenyl)pyrimidin-2-yl)amino)cyclohexyl)acetamide was consumed completely and the desired mass was detected. The reaction mixture was diluted with H₂O 20 mL and extracted with EtOAc (20 mL*3). The combined organic layers were concentrated under reduced pressure. The residue was purified by prep-HPLC (column: Phenomenex Luna C18 75*30 mm*3 um; mobile phase: [H₂O (0.2% FA)-ACN]; gradient: 10%-40% B over 8.0 min) to give the title compound (Compound 369) (7.80 mg, 8.63 μmol, 7.43% yield, 98.5% purity, 0.62 FA) as a white solid. LCMS (ES⁺): m/z 862.3 [M+H]⁺.

### Example 407. Synthesis of 3-(6-(1-((1-(2-(4-((5-chloro-4-(3-(2-oxopyridin-1(2H)-yl)phenyl)pyrimidin-2-yl)amino)piperidin-1-yl)-2-oxoethyl)piperidin-4-yl)methyl)piperidin-4-yl)-2-oxobenzo[d]oxazol-3 (2H)-yl)piperidine-2,6-dione (Compound 373)

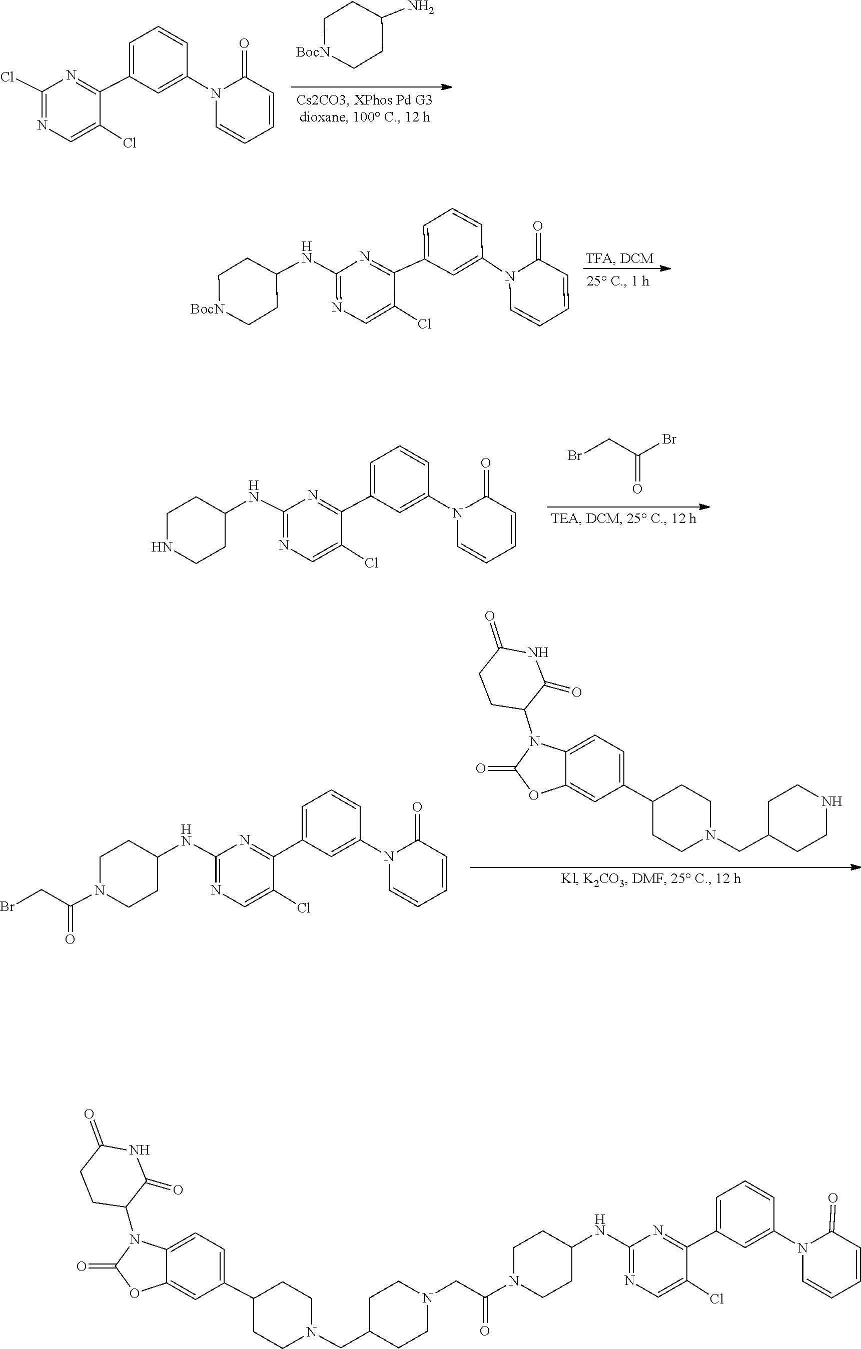

Synthesis of tert-butyl 4-((5-chloro-4-(3-(2-oxopyridin-1(2H)-yl)phenyl)pyrimidin-2-yl)amino)piperidine-1-carboxylate. A mixture of 1-[3-(2,5-dichloropyrimidin-4-yl)phenyl]pyridin-2-one (700 mg, 2.20 mmol, 1.00 eq), tert-butyl 4-aminopiperidine-1-carboxylate (660 mg, 3.30 mmol, 1.50 eq), Cs₂CO₃ (2.15 g, 6.60 mmol, 3.00 eq) and XPhosPdG3 (186 mg, 220 μmol, 0.10 eq) in dioxane (10 mL) was degassed and purged with N₂ for 3 times at 25 °C, and then the mixture was stirred at 100 °C for 12 h under N₂ atmosphere. LC-MS showed the desired mass was detected. The reaction mixture was diluted with H₂O 30 mL and extracted with EtOAc (30 mL*3). The combined organic layers were concentrated under reduced pressure. The residue was purified by column chromatography (SiO₂, Petroleum ether:Ethyl acetate=0:1 to 1:0) to give the title compound (430 mg, 734 μmol, 33.4% yield, 82.4% purity) as a yellow solid. LCMS (ES⁺): m/z 482.2 [M+H]⁺.

Synthesis of 1-(3-(5-chloro-2-(piperidin-4-ylamino)pyrimidin-4-yl)phenyl)pyridin-2(1H)-one. To a solution of tert-butyl 4-[[5-chloro-4-[3-(2-oxo-1-pyridyl)phenyl]pyrimidin-2-yl]amino]piperidine-1-carboxylate (150 mg, 311 μmol, 1.00 eq) in DCM (6 mL) was added TFA (3.07 g, 26.9 mmol, 2.00 mL, 86.5 eq) at 25 °C. The mixture was stirred at 25 °C for 1 h. LC-MS showed the desired mass was detected. The reaction was concentrated under reduced pressure to give the title compound (120 mg, crude, TFA) was obtained as a yellow oil. LCMS (ES⁺): m/z 382.2 [M+H]⁺.

Synthesis of 1-(3-(2-((1-(2-bromoacetyl)piperidin-4-yl)amino)-5-chloropyrimidin-4-yl)phenyl)pyridin-2(1H)-one. To a solution of 1-(3-(5-chloro-2-(piperidin-4-ylamino)pyrimidin-4-yl)phenyl)pyridin-2(1H)-one (100 mg, 239 μmol, 1.00 eq, HCl) in DCM (2 mL) was added TEA (72.5 mg, 717 μmol, 99.8 μL, 3.00 eq) and 2-bromoacetyl bromide (48.2 mg, 239 μmol, 20.8 μL, 1.00 eq) at 25 °C. The mixture was stirred at 25 °C for 12 h. LC-MS showed the desired mass was detected. The reaction was concentrated under reduced pressure to give the title compound (100 mg, crude) as a yellow oil. LCMS (ES⁺): m/z 502.2 [M+H]⁺.

Synthesis of 3-(6-(1-((1-(2-(4-((5-chloro-4-(3-(2-oxopyridin-1(2H)-yl)phenyl)pyrimidin-2-yl)amino)piperidin-1-yl)-2-oxoethyl)piperidin-4-yl)methyl)piperidin-4-yl)-2-oxobenzo[d]oxazol-3 (2H)-yl)piperidine-2,6-dione (Compound 373). To a solution of 1-(3-(2-((1-(2-bromoacetyl)piperidin-4-yl)amino)-5-chloropyrimidin-4-yl)phenyl)pyridin-2(1H)-one (100 mg, 198 μmol, 1.00 eq) in DMF (5 mL) was added 3-(2-oxo-6-(1-(piperidin-4-ylmethyl)piperidin-4-yl)benzo[d]oxazol-3 (2H)-yl)piperidine-2,6-dione (107 mg, 198 μmol, 1.00 eq, TFA), K₂CO₃ (54.9 mg, 397 μmol, 2.00 eq) and KI (33.0 mg, 198 μmol, 1.00 eq) at 25 °C. The mixture was stirred at 25 °C for 12 h. LC-MS showed the desired mass was detected. The reaction mixture was diluted with H₂O 20 mL and extracted with EtOAc (20 mL*3). The combined organic layers were concentrated under reduced pressure. The residue was purified by prep-HPLC (column: Phenomenex Luna C18 80*30 mm*3 um; mobile phase: [H₂O (0.1% TFA)-ACN]; gradient: 5%-35% B over 8.0 min). (column: Phenomenex Luna C18 100*30 mm*3 um; mobile phase: [H₂O (0.2% FA)-ACN]; gradient: 15%-55% B over 8.0 min) to give the title compound (Compound 373) (2.30 mg, 2.68 μmol, 1.35% yield, 98.7% purity) as a white solid. LCMS (ES⁺): m/z 848.3 [M+H]⁺.

### Example 408. Synthesis of 3-[6-[1-[[1-[2-[4-[[5-fluoro-4-[3-(2-oxo-1H-pyridin-3-yl)phenyl]pyrimidin-2-yl]amino]-1-piperidyl]-2-oxo-ethyl]-4-piperidyl]methyl]-4-piperidyl]-2-oxo-1,3-benzoxazol-3-yl]piperidine-2,6-dione (Compound 379)

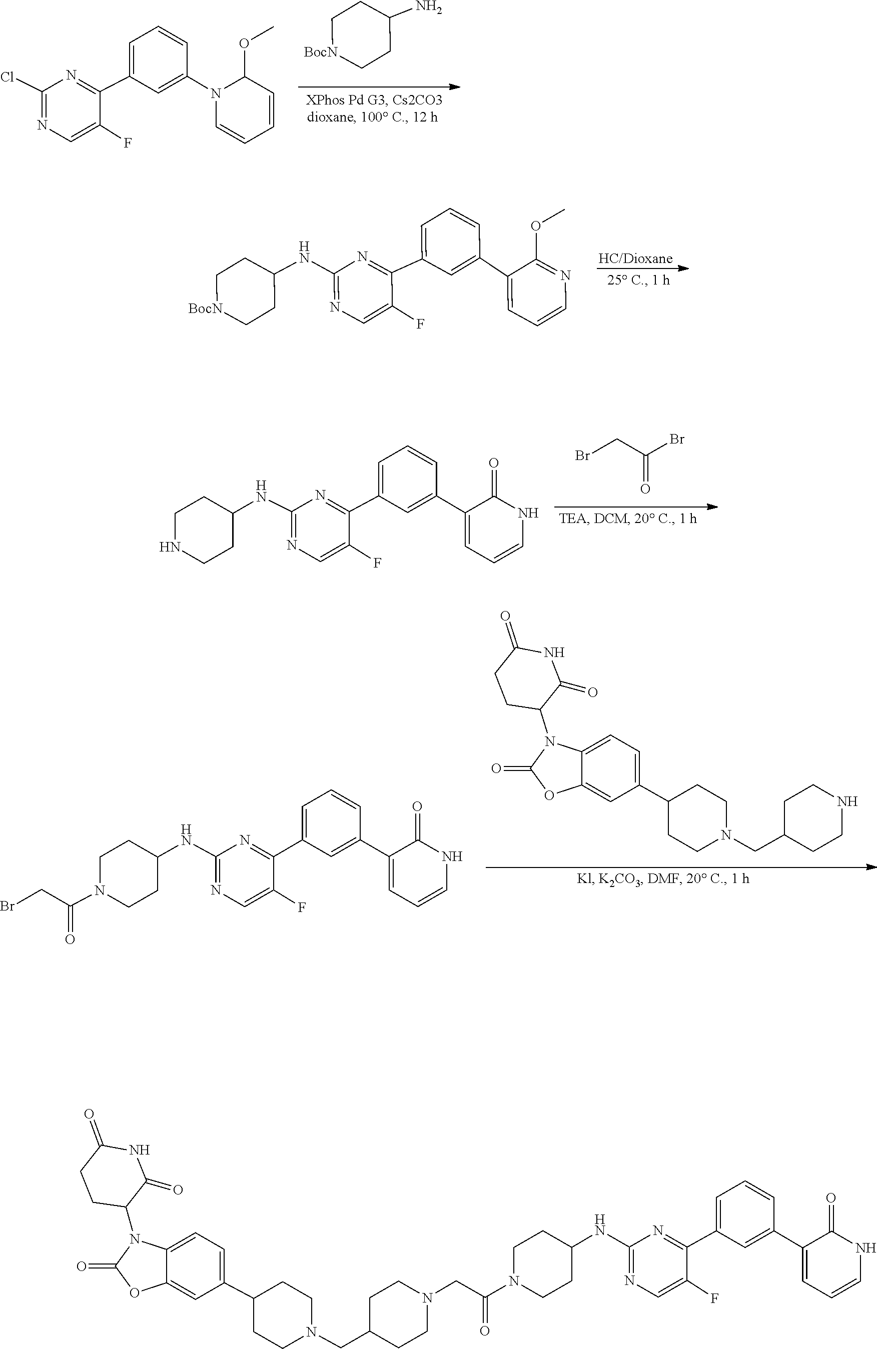

Synthesis of tert-butyl 4-[[5-fluoro-4-[3-(2-methoxy-3-pyridyl)phenyl]pyrimidin-2-yl]amino]piperidine-1-carboxylate. A mixture of 2-chloro-5-fluoro-4-[3-(2-methoxy-3-pyridyl)phenyl]pyrimidine (700 mg, 2.22 mmol, 1.00 eq), tert-butyl 4-aminopiperidine-1-carboxylate (666 mg, 3.33 mmol, 1.50 eq), Cs₂CO₃ (2.17 g, 6.65 mmol, 3.00 eq), XPhos Pd G3 (187 mg, 221 μmol, 0.100 eq) in Dioxane (5 mL) was degassed and purged with N₂ for 3 times at 20 °C, and then the mixture was stirred at 100 °C for 12 h under N₂ atmosphere. LC-MS showed the desired mass was detected. The reaction mixture was quenched by addition H₂O 10 mL, and then extracted with EtOAc (10 mL*3). The combined organic layers were dried over Na₂SO₄, filtered and concentrated under reduced pressure. The residue was purified by column chromatography (SiO₂, Petroleum ether:Ethyl acetate=20:1 to 3:1) to give the title compound (350 mg, crude) as a yellow solid. LCMS (ES⁺): m/z 480.4[M+H]⁺.

Synthesis of 3-[3-[5-fluoro-2-(4-piperidylamino) pyrimidin-4-yl]phenyl]-1H-pyridin-2-one. To a solution of tert-butyl 4-[[5-fluoro-4-[3-(2-methoxy-3-pyridyl)phenyl]pyrimidin-2-yl]amino]piperidine-1-carboxylate (300 mg, 625 μmol, 1.00 eq) in Dioxane (0.5 mL) was added HCl (4.00 M, 6.99 mL, 44.7 eq) at 20 °C. The mixture was stirred at 80 °C for 12 h. LC-MS showed the desired mass was detected. The reaction mixture was concentrated under high vacuum to give the title compound (120 mg, crude, HCl) as a white solid. LCMS (ES⁺): m/z 366.0 [M+H]⁺.

Synthesis of 3-[3-[2-[[1-(2-bromoacetyl)-4-piperidyl]amino]-5-fluoro-pyrimidin-4-yl]phenyl]-1H-pyridin-2-one. To a solution of 3-[3-[5-fluoro-2-(4-piperidylamino)pyrimidin-4-yl]phenyl]-1H-pyridin-2-one (100 mg, 248 μmol, 1.00 eq, HCl) in DCM (2 mL) was added 2-bromoacetyl bromide (100 mg, 497 μmol, 43.3 μL, 2.00 eq) and DIEA (96.4 mg, 746 μmol, 130 μL, 3.00 eq) at 20 °C. The mixture was stirred at 20 °C for 1 h. LC-MS showed the desired mass was detected. The reaction mixture was concentrated under reduced pressure. The residue was purified by prep-TLC (SiO₂, Petroleum ether:Ethyl acetate=3:1) to give the title compound (120 mg, crude) as a yellow solid. LCMS (ES⁺): m/z 486.1 [M+H]⁺.

Synthesis of 3-[6-[1-[[1-[2-[4-[[5-fluoro-4-[3-(2-oxo-1H-pyridin-3-yl)phenyl]pyrimidin-2-yl]amino]-1-piperidyl]-2-oxo-ethyl]-4-piperidyl]methyl]-4-piperidyl]-2-oxo-1,3-benzoxazol-3-yl]piperidine-2,6-dione (Compound 379). A mixture of 3-[3-[2-[[1-(2-bromoacetyl)-4-piperidyl]amino]-5-fluoro-pyrimidin-4-yl]phenyl]-1H-pyridin-2-one (105 mg, 215 μmol, 1.00 eq), 3-(2-oxo-6-(1-(piperidin-4-ylmethyl)piperidin-4-yl)benzo[d]oxazol-3 (2H)-yl)piperidine-2,6-dione (92.0 mg, 170 μmol, 0.781 eq, TFA), KI (35.8 mg, 215 μmol, 1.00 eq), K₂CO₃ (89.5 mg, 647 μmol, 3.00 eq) in DMF (2 mL) was degassed and purged with N₂ for 3 times, and then the mixture was stirred at 20 °C for 1 h under N₂ atmosphere. LC-MS showed the desired mass was detected. The reaction mixture was quenched by addition H₂O 10 mL, and then extracted with EtOAc (10 mL*3). The combined organic layers were dried over Na₂SO₄, filtered and concentrated under reduced pressure. The residue was purified by prep-HPLC (column: Phenomenex luna C18 100*40 mm*3 um; mobile phase: [H₂O (0.2% FA)-ACN]; gradient: 1%-30% B over 8.0 min) to give the title compound (Compound 379) (44.3 mg, 49.8 μmol, 23.1% yield, 99.3% purity, 1.1 FA) as a off-white solid. LCMS (ES⁺): m/z 832.5 [M+H]⁺.

### Example 409. Synthesis of 3-((3-fluoro-4-(1-((1-(2-(4-((5-fluoro-4-(3-(2-oxo-1,2-dihydropyridin-3-yl)phenyl)pyrimidin-2-yl)amino)piperidin-1-yl)-2-oxoethyl)piperidin-4-yl)methyl)piperidin-4-yl)phenyl)amino)piperidine-2,6-dione (Compound 364)

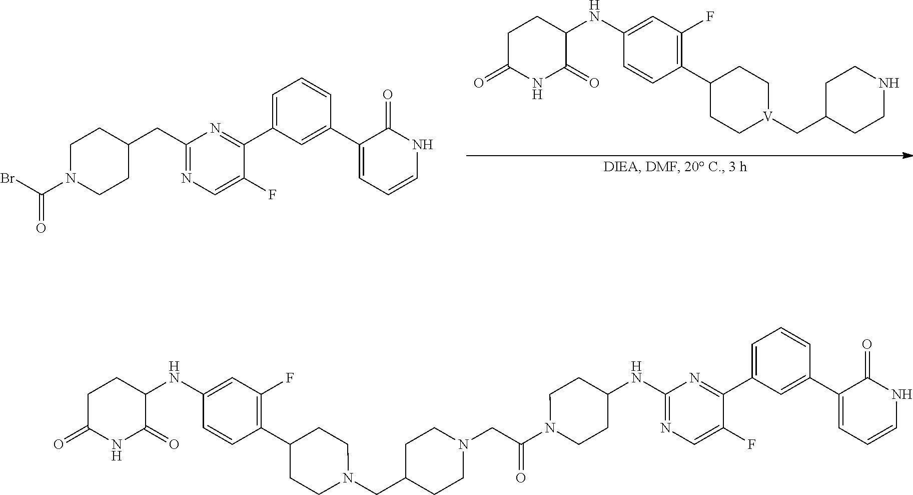

Synthesis of 3-((3-fluoro-4-(1-((1-(2-(4-((5-fluoro-4-(3-(2-oxo-1,2-dihydropyridin-3-yl)phenyl)pyrimidin-2-yl)amino)piperidin-1-yl)-2-oxoethyl)piperidin-4-yl)methyl)piperidin-4-yl)phenyl)amino)piperidine-2,6-dione (Compound 364). To a solution of 3-((3-fluoro-4-(1-(piperidin-4-ylmethyl)piperidin-4-yl)phenyl)amino)piperidine-2,6-dione (637 mg, 1.23 mmol, 1.50 eq, TFA) in DMF (10 mL) were added DIEA (319 mg, 2.47 mmol, 430 μL, 3.00 eq) and 3-(3-(2-((1-(2-bromoacetyl)piperidin-4-yl)amino)-5-fluoropyrimidin-4-yl)phenyl)pyridin-2(1H)-one (400 mg, 822 μmol, 1.00 eq) at 20 °C. The mixture was stirred at 20 °C for 3 h. LCMS showed 3-(3-(2-((1-(2-bromoacetyl)piperidin-4-yl)amino)-5-fluoropyrimidin-4-yl)phenyl)pyridin-2(1H)-one was consumed and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min]; gradient: 30%-60% B over 8.0 min) to give the title compound (Compound 364) (40.8 mg, 50.2 μmol, 6.11% yield, 99.5% purity) as a white solid. LCMS (ES⁺): m/z 808.4 [M+H]⁺

### Example 410. Synthesis of 3-(6-(1-(2-(1-(trans-4-((5-fluoro-4-(3-(2-oxo-1,2-dihydropyridin-3-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 368)

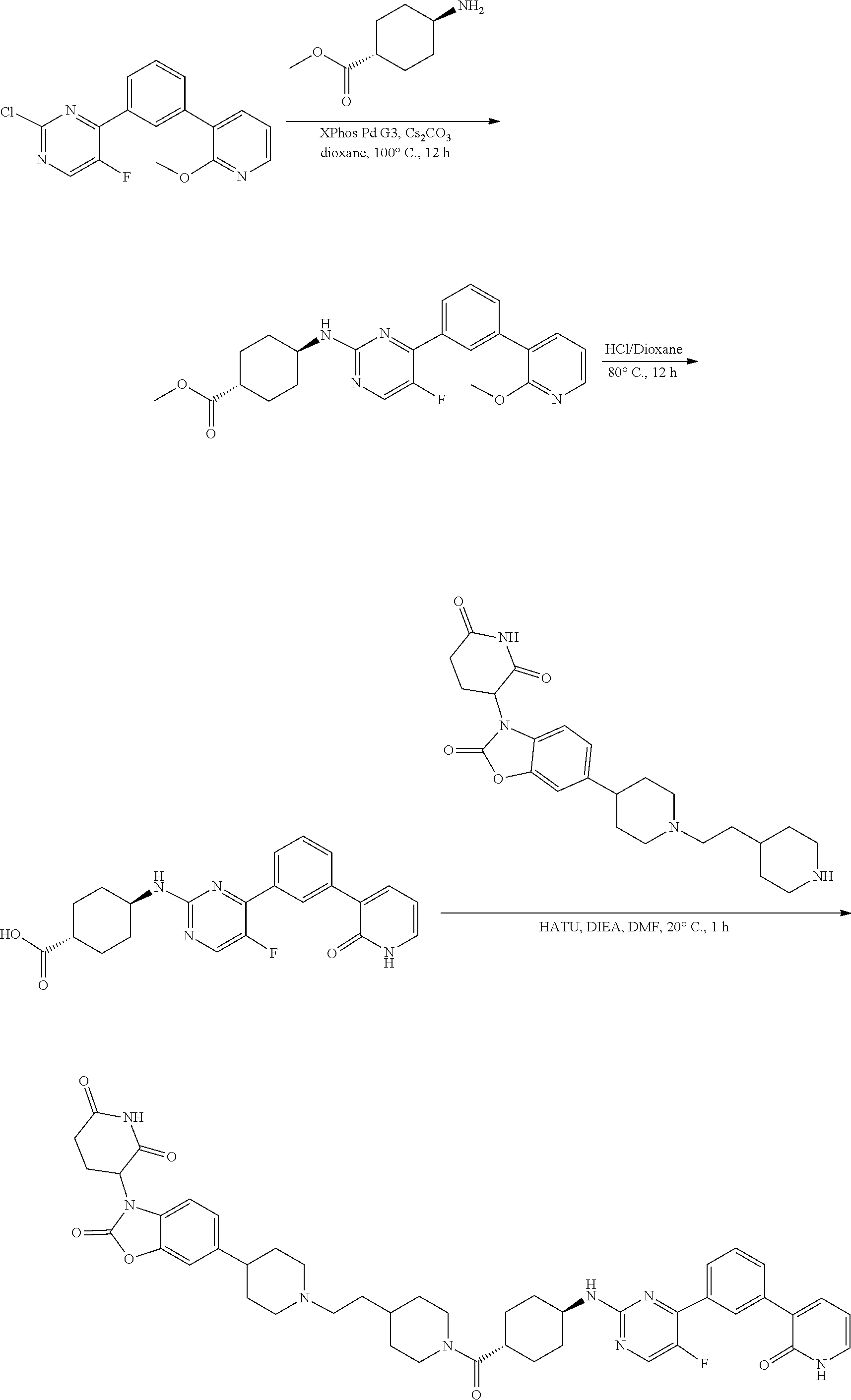 [SMILES: COc1ncccc1-c1cccc(-c2nc(Cl)ncc2F)c1 ; COC(=O)[C@H]1CC[C@H](N)CC1 ; COc1ncccc1-c1cccc(-c2nc(N[C@H]3CC[C@H](C(=O)OC)CC3)ncc2F)c1 ; O=c1[nH]cccc1-c1cccc(-c2nc(N[C@H]3CC[C@H](C(=O)O)CC3)ncc2F)c1 ; O=C1CCC(n2c(=O)oc3cc(C4CCN(CCC5CCNCC5)CC4)ccc32)C(=O)N1 ; O=C1CCC(n2c(=O)oc3cc(C4CCN(CCC5CCN(C(=O)[C@H]6CC[C@H](Nc7ncc(F)c(-c8cccc(-c9ccc[nH]c9=O)c8)n7)CC6)CC5)CC4)ccc32)C(=O)N1]

Synthesis of methyl-trans-4-((5-fluoro-4-(3-(2-methoxypyridin-3-yl)phenyl)pyrimidin-2-yl)amino)cyclohexane-1-carboxylate. A mixture of 2-chloro-5-fluoro-4-(3-(2-methoxypyridin-3-yl)phenyl)pyrimidine (650 mg, 2.06 mmol, 1.00 eq), methyl-trans-4-aminocyclohexane-1-carboxylate (485 mg, 3.09 mmol, 1.50 eq), Cs₂CO₃ (1.34 g, 4.12 mmol, 2.00 eq), XPhos Pd G3 (174 mg, 205 μmol, 0.10 eq) in Dioxane (10 mL) was degassed and purged with N₂ for 3 times at 20 °C, and then the mixture was stirred at 100 °C for 12 h under N₂ atmosphere. LC-MS showed 2-chloro-5-fluoro-4-(3-(2-methoxypyridin-3-yl)phenyl)pyrimidine was consumed completely and the desired mass was detected. The reaction mixture was quenched by addition H₂O 20 mL, and then extracted with EtOAc (20 mL*3). The combined organic layers were dried over Na₂SO₄, filtered and concentrated under reduced pressure. The residue was purified by column chromatography (SiO₂, Petroleum ether:Ethyl acetate=20:1 to 3:1) to give the title compound (450 mg, crude) as a yellow solid. LCMS (ES⁺): m/z 437.2 [M+H]⁺.

Synthesis of trans-4-((5-fluoro-4-(3-(2-oxo-1,2-dihydropyridin-3-yl)phenyl)pyrimidin-2-yl)amino)cyclohexane-1-carboxylic acid. To a solution of methyl-trans-4-((5-fluoro-4-(3-(2-methoxypyridin-3-yl)phenyl)pyrimidin-2-yl)amino)cyclohexane-1-carboxylate (50.0 mg, 114 μmol, 1.00 eq) in dioxane (0.5 mL) was added HCl (12 M, 9.55 μL, 1.00 eq) at 20 °C. The mixture was stirred at 80 °C for 12 h. LC-MS showed the desired mass was detected. The reaction mixture was concentrated under high vacuum to give the title compound (40.0 mg, crude, HCl) as a yellow solid. LCMS (ES⁺): m/z 409.3 [M+H]⁺.

Synthesis of 3-(6-(1-(2-(1-(trans-4-((5-fluoro-4-(3-(2-oxo-1,2-dihydropyridin-3-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 368). A mixture of trans-4-((5-fluoro-4-(3-(2-oxo-1,2-dihydropyridin-3-yl)phenyl)pyrimidin-2-yl)amino)cyclohexane-1-carboxylic acid (35.0 mg, 85.7 μmol, 1.00 eq, HCl), 3-(2-oxo-6-(1-(2-(piperidin-4-yl)ethyl)piperidin-4-yl)benzo[d]oxazol-3 (2H)-yl)piperidine-2,6-dione (37.7 mg, 85.7 μmol, 1.00 eq, TFA), HATU (97.7 mg, 257 μmol, 3.00 eq), DIEA (22.1 mg, 171 μmol, 29.8 μL, 2.00 eq) in DMF (1 mL) was degassed and purged with N₂ for 3 times, and then the mixture was stirred at 20 °C for 1 h under N₂ atmosphere. LC-MS showed the desired mass was detected. The residue was purified by prep-HPLC (column: 3_Phenomenex Luna C18 75*30 mm*3 um; mobile phase: [H₂O (0.1% TFA)-ACN]; gradient: 15%-45% B over 8.0 min) to give the title compound (Compound 368) (4.10 mg, 4.83 μmol, 8.03% yield, 97.9% purity) as a brown solid. LCMS (ES⁺): m/z 831.4 [M+H]⁺.

### Example 411. Synthesis of trans-(1r,4R)—N-((1r,4R)-4-(4-(4-((2,6-dioxopiperidin-3-yl)amino)-2-fluorophenyl)piperidin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 402)

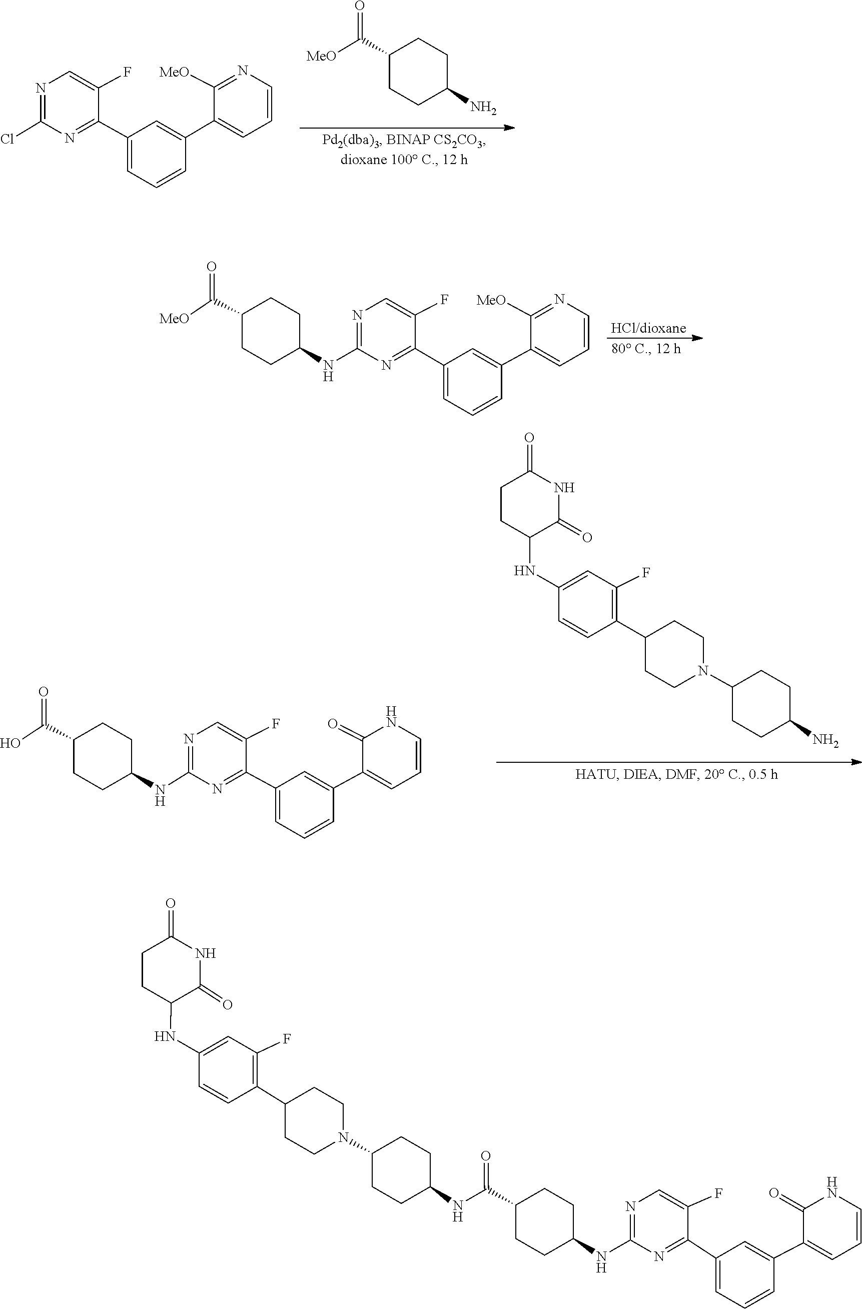

Synthesis of trans-methyl 4-((5-fluoro-4-(3-(2-methoxypyridin-3-yl)phenyl)pyrimidin-2-yl)amino)cyclohexane-1-carboxylate. To a solution of 2-chloro-5-fluoro-4-(3-(2-methoxypyridin-3-yl)phenyl)pyrimidine (12.0 g, 38.0 mmol, 1.00 eq) and trans-methyl 4-aminocyclohexanecarboxylate (8.96 g, 57.0 mmol, 1.50 eq) in dioxane (300 mL) was added Cs₂CO₃ (37.2 g, 114 mmol, 3.00 eq), BINAP (1.18 g, 1.90 mmol, 0.0500 eq) and Pd₂(dba)₃ (1.74 g, 1.90 mmol, 0.0500 eq) at 20 °C. The resulting mixture was stirred at 100 °C for 12 h under N₂. LC-MS showed 2-chloro-5-fluoro-4-(3-(2-methoxypyridin-3-yl)phenyl)pyrimidine was consumed completely and the desired mass was detected. The mixture was diluted with H₂O (300 mL) and then extracted with EtOAc (200 mL*3). The combined organic layers were dried over Na₂SO₄, filtered and the filtrate was concentrated in vacuo to give a residue. The residue was purified by column chromatography (SiO₂, PE/EtOAc=1/0 to 0/1) to give the title compound (10.0 g, 22.9 mmol, 60.3% yield) as a yellow solid. LCMS (ES⁺): m/z 437.1 [M+H]⁺

Synthesis of trans-4-((5-fluoro-4-(3-(2-oxo-1,2-dihydropyridin-3-yl)phenyl)pyrimidin-2-yl)amino)cyclohexane-1-carboxylic acid. A stirred solution of trans-methyl 4-((5-fluoro-4-(3-(2-methoxypyridin-3-yl)phenyl)pyrimidin-2-yl)amino)cyclohexane-1-carboxylate (10.0 g, 22.91 mmol, 1.00 eq) in dioxane (70 mL) and HCl (35 mL) was stirred at 80 °C for 12 h. LC-MS showed trans-methyl 4-((5-fluoro-4-(3-(2-methoxypyridin-3-yl)phenyl)pyrimidin-2-yl)amino)cyclohexane-1-carboxylate was consumed completely and the desired mass was detected. The mixture was adjust pH to 8-9 by aqueous LiOH solution (1M). The mixture was washed with EtOAc (50 mL*3), then the aqueous layer was adjust pH to 4-5 by HCl (1M). The mixture was filtered and the filter cake was dried in vacuo to give the title compound (9.00 g, crude) as a brown solid. LCMS (ES⁺): m/z 409.1 [M+H]⁺

Synthesis of trans-(1r,4R)—N-((1r,4R)-4-(4-(4-((2,6-dioxopiperidin-3-yl)amino)-2-fluorophenyl)piperidin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 402). To a solution of trans-3-[4-[1-(4-aminocyclohexyl)-4-piperidyl]-3-fluoro-anilino]piperidine-2,6-dione (490 mg, 1.22 mmol, 1 eq) and trans-4-((5-fluoro-4-(3-(2-oxo-1,2-dihydropyridin-3-yl)phenyl)pyrimidin-2-yl)amino)cyclohexane-1-carboxylic acid (248.60 mg, 608.69 μmol, 0.5 eq) in DMF (4 mL) were added HATU (462.88 mg, 1.22 mmol, 1.00 eq) and DIEA (472.00 mg, 3.65 mmol, 636.12 μL, 3.00 eq) at 20 °C, the mixture was stirred at 20 °C for 0.5 h. LCMS showed trans-4-((5-fluoro-4-(3-(2-oxo-1,2-dihydropyridin-3-yl)phenyl)pyrimidin-2-yl)amino)cyclohexane-1-carboxylic acid was consumed completely and the desired mass was detected. The reaction was filtered and the filtrate was concentrated under reduced pressure to give a residue. The residue was purified by prep-HPLC (column: Phenomenex Luna C18 100*30 mm*3 um; mobile phase: [H₂O (0.04% HCl)-ACN]; gradient: 15%-45% B over 8.0 min) to give the title compound (Compound 402) (62.0 mg, 74.4 μmol, 6.11% yield, 95.2% purity) as a yellow solid. LCMS (ES⁺): m/z 793.4 [M+H]⁺

### Example 412. Synthesis of trans-(1r,4R)—N-((1R,4R)-4-(4-(4-(((R)-2,6-dioxopiperidin-3-yl)amino)-2-fluorophenyl)piperidin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 419) and trans-(1r,4R)—N-((1S,4R)-4-(4-(4-(((S)-2,6-dioxopiperidin-3-yl)amino)-2-fluorophenyl)piperidin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 435) (The Two Isomers were Assigned Arbitrarily)

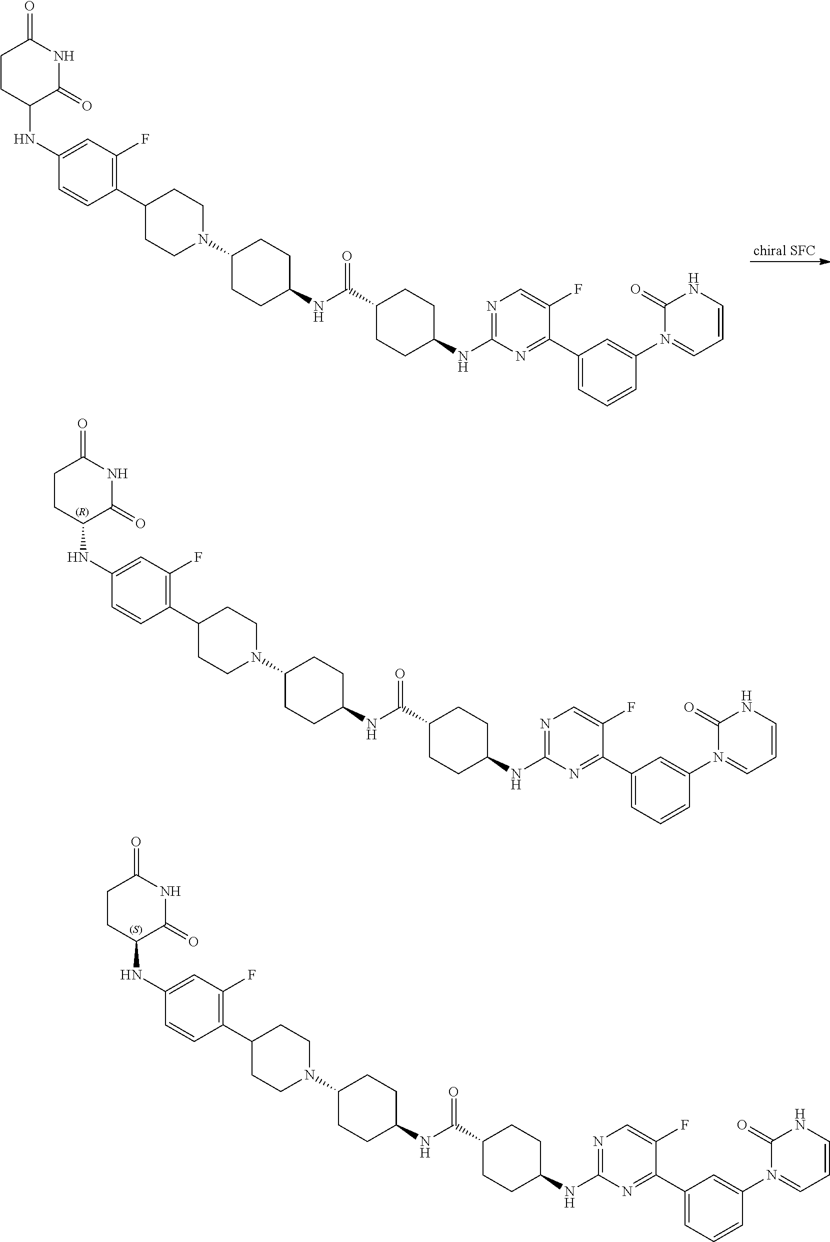

Synthesis of trans-(1r,4R)—N-((1R,4R)-4-(4-(4-(((R)-2,6-dioxopiperidin-3-yl)amino)-2-fluorophenyl)piperidin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 419) and trans-(1r,4R)—N-((1S,4R)-4-(4-(4-(((S)-2,6-dioxopiperidin-3-yl)amino)-2-fluorophenyl)piperidin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 435) (the two isomers were assigned arbitrarily). Trans-(1r,4R)—N-((1r,4R)-4-(4-(4-((2,6-dioxopiperidin-3-yl)amino)-2-fluorophenyl)piperidin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 402) (40 mg, 50.5 umol, 1.00 eq) was separated by chiral SFC (column: REGIS(S,S)WHELK-O1 (250 mm*25 mm, 10 um); mobile phase: [CO₂-IPA (0.1% IPAm)]; B %: 56%, isocratic elution mode) to give the title compound (Compound 419) (12.3 mg, 14.62 μmol, 28.99% yield, 94.27% purity, 100% ee) (retention time: 1.422, peak 1) as a green solid. LCMS (ES+): m/z 793.3 [M+H]⁺ and the title compound (Compound 435) (10.4 mg, 13.01 μmol, 25.80% yield, 99.22% purity, 94.7% ee) (retention time: 1.826, peak 2) as a yellow solid. LCMS (ES+): m/z 793.3 [M+H]⁺.

### Example 413. Synthesis of trans-N-(trans-4-(4-(4-((2,6-dioxopiperidin-3-yl)amino)-2-fluorophenyl)piperazin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 382

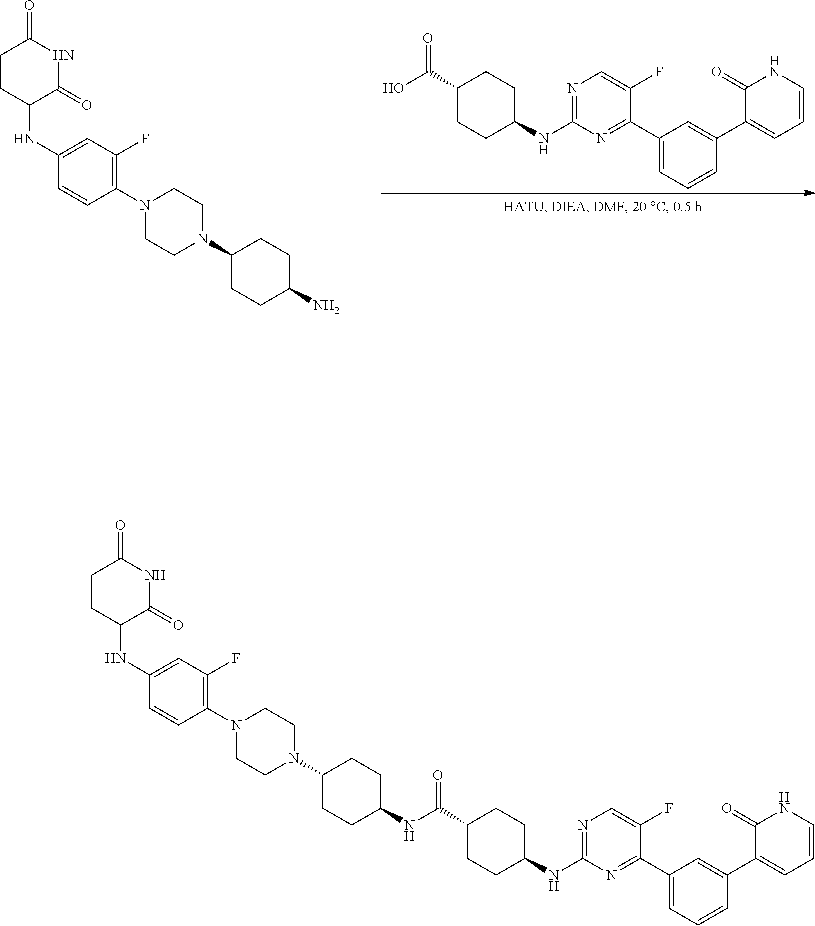 [SMILES: N[C@H]1CC[C@@H](N2CCN(c3ccc(NC4CCC(=O)NC4=O)cc3F)CC2)CC1 ; O=c1[nH]cccc1-c1cccc(-c2nc(N[C@H]3CC[C@H](C(=O)O)CC3)ncc2F)c1 ; O=C1CCC(Nc2ccc(N3CCN([C@H]4CC[C@H](NC(=O)[C@H]5CC[C@H](Nc6ncc(F)c(-c7cccc(-c8ccc[nH]c8=O)c7)n6)CC5)CC4)CC3)c(F)c2)C(=O)N1]

Synthesis of trans-N-(trans-4-(4-(4-((2,6-dioxopiperidin-3-yl)amino)-2-fluorophenyl)piperazin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 382). To a solution of trans-(1r,4r)-4-((5-fluoro-4-(3-(2-oxo-1,2-dihydropyridin-3-yl)phenyl)pyrimidin-2-yl)amino)cyclohexane-1-carboxylic acid (430 mg, 966 μmol, 0.500 eq, HCl) in DMF (20 mL) were added HATU (257 mg, 676 μmol, 0.350 eq), DIEA (749 mg, 5.80 mmol, 1.01 mL, 3.00 eq) and trans-3-((4-(4-((1r,4r)-4-aminocyclohexyl)piperazin-1-yl)-3-fluorophenyl)amino)piperidine-2,6-dione (1.00 g, 1.93 mmol, 1.00 eq, TFA) at 25 °C. The mixture was stirred at 25 °C for 0.5 h. LC-MS showed trans-3-((4-(4-((1r,4r)-4-aminocyclohexyl)piperazin-1-yl)-3-fluorophenyl)amino)piperidine-2,6-dione was consumed completely, and the desired mass was detected. The reaction mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 1%-40% B over 8.0 min) to give the title compound (Compound 382) (178 mg, 219 μmol, 11.3% yield, 97.4% purity) as a brown solid. LCMS (ES⁺): m/z 794.4 [M+H]⁺.

### Example 414. Synthesis of trans-(1r,4R)—N-((1R,4R)-4-(4-(4-(((R)-2,6-dioxopiperidin-3-yl)amino)-2-fluorophenyl)piperazin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 432) and trans-(1r,4R)—N-((1S,4R)-4-(4-(4-(((S)-2,6-dioxopiperidin-3-yl)amino)-2-fluorophenyl)piperazin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 441) (The Two Isomers were Assigned Arbitrarily)

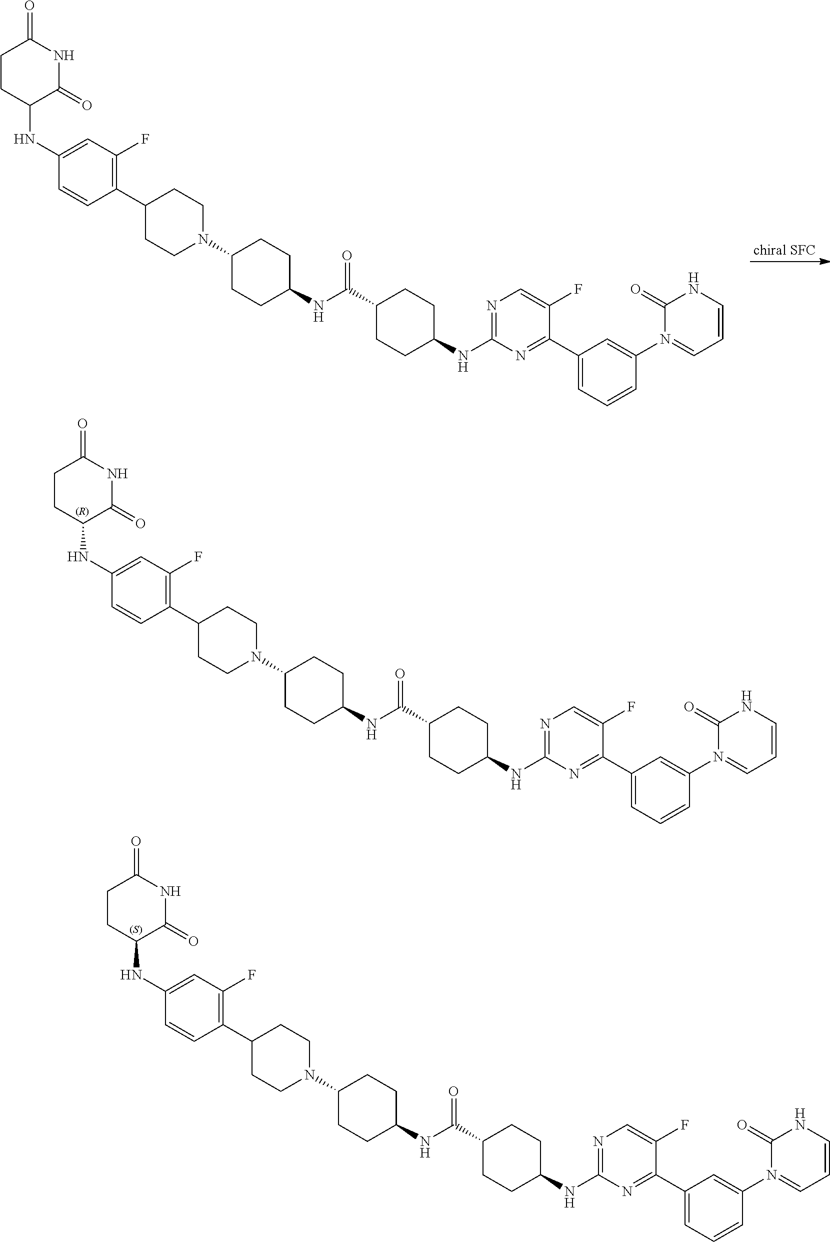

Synthesis of trans-(1r,4R)—N-((1R,4R)-4-(4-(4-(((R)-2,6-dioxopiperidin-3-yl)amino)-2-fluorophenyl)piperazin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 432) and trans-(1r,4R)—N-((1S,4R)-4-(4-(4-(((S)-2,6-dioxopiperidin-3-yl)amino)-2-fluorophenyl)piperazin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 441) (the two isomers were assigned arbitrarily). Trans-(1r,4R)—N-((1r,4R)-4-(4-(4-((2,6-dioxopiperidin-3-yl)amino)-2-fluorophenyl)piperazin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 382) (170 mg, 214 μmol, 1.00 eq) was further separated by SFC (column: REGIS(S,S)WHELK-O1 (250 mm*25 mm, 10 um); mobile phase: [CO₂-IPA (0.1% IPAm)]; B %: 56%, isocratic elution mode) to give the title compound (Compound 432) (44.1 mg, 51.7 μmol, 24.2% yield, 93.1% purity, 97.6% ee) (retention time: 1.36, peak 1) as a white solid. LCMS (ES⁺): m/z 794.4 [M+H]+ and give the title compound (Compound 441) (12.6 mg, 15.9 μmol, 7.41% yield, 100% purity, 93.8% ee) (retention time: 1.50, peak 2) as a pale yellow solid. LCMS (ES⁺): m/z 794.4 [M+H]⁺.

### Example 415. Synthesis of trans-3-((3-fluoro-4-(4-((1-(4-((5-fluoro-4-(3-(2-oxo-1,2-dihydropyridin-3-yl)phenyl)pyrimidin-2-yl)amino)cyclohexane-1-carbonyl)piperidin-4-yl)oxy)piperidin-1-yl)phenyl)amino)piperidine-2,6-dione (Compound 403)

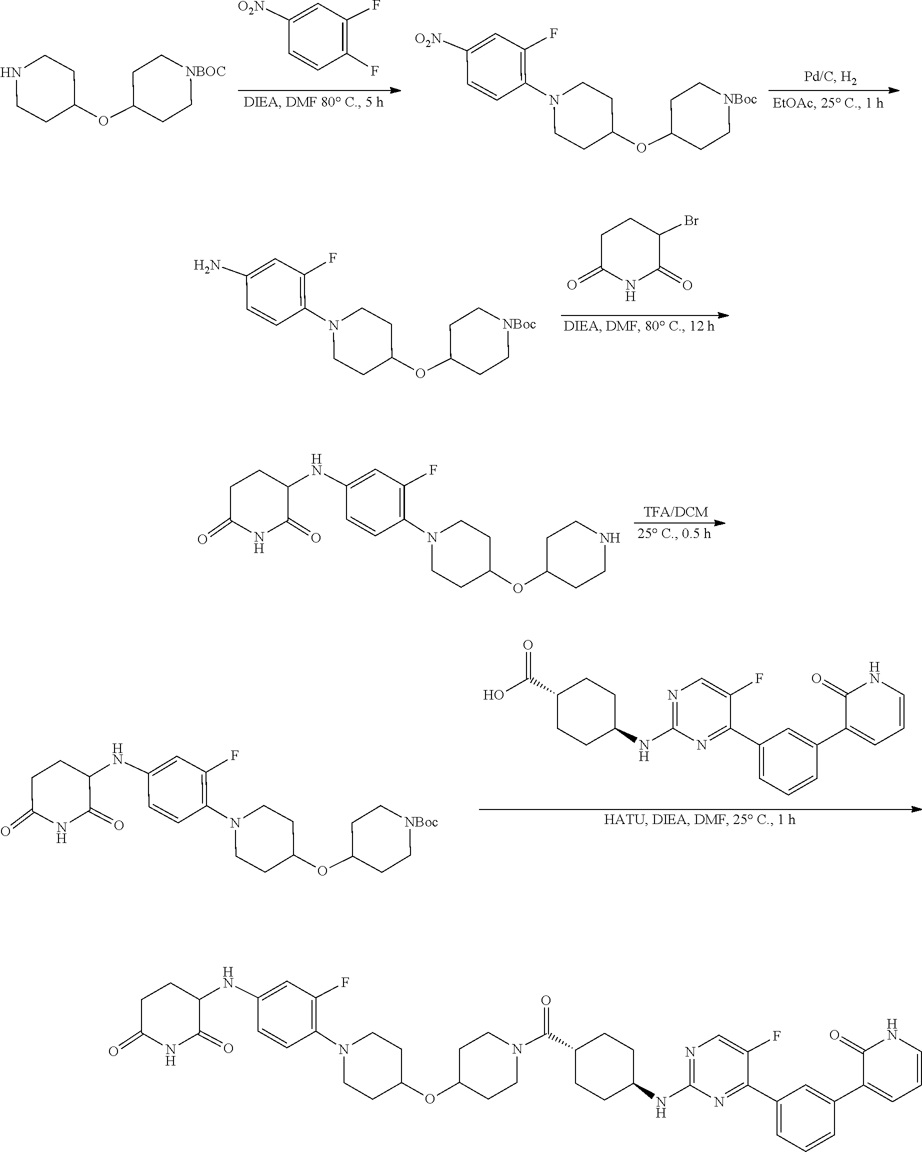

Synthesis of tert-butyl 4-((1-(2-fluoro-4-nitrophenyl)piperidin-4-yl)oxy)piperidine-1-carboxylate. To a solution of tert-butyl 4-(piperidin-4-yloxy)piperidine-1-carboxylate (5.00 g, 17.6 mmol, 1.00 eq) and 1,2-difluoro-4-nitro-benzene (2.80 g, 17.6 mmol, 1.95 mL, 1.00 eq) in DMF (40 mL) was added DIEA (13.6 g, 105 mmol, 18.4 mL, 6.00 eq) at 25 °C. The mixture was stirred at 80 °C for 5 h. LCMS showed tert-butyl 4-(piperidin-4-yloxy)piperidine-1-carboxylate was consumed completely and the desired mass was detected. Water (20 mL) was added to the reaction. The reaction mixture was filtered and the filter cake was dried in vacuum to give the title compound (12.5 g, crude) as a bright yellow solid. LCMS (ES+): m/z 424.2 [M+H]⁺

Synthesis of tert-butyl 4-((1-(4-amino-2-fluorophenyl)piperidin-4-yl)oxy)piperidine-1-carboxylate. 10% Pd/C (15.0 g, 50.0% purity) was added to EtOAc (300 mL) at 25 °C under N₂ atmosphere, then tert-butyl 4-((1-(2-fluoro-4-nitrophenyl)piperidin-4-yl)oxy)piperidine-1-carboxylate (12.5 g, 29.5 mmol, 1.00 eq) was added at 25 °C, the suspension was degassed and purged with H₂ for three times. The mixture was stirred at 25 °C for 1 h under H₂ (15 psi) atmosphere. LCMS showed tert-butyl 4-((1-(2-fluoro-4-nitrophenyl)piperidin-4-yl)oxy)piperidine-1-carboxylate was consumed completely and the desired mass was detected. The mixture was filtered and the filtrate was concentrated under reduced pressure to give the title compound (11.1 g, crude) as a yellow oil. LCMS (ES+): m/z 394.1 [M+H]⁺.

Synthesis of tert-butyl 4-((1-(4-((2,6-dioxopiperidin-3-yl)amino)-2-fluorophenyl)piperidin-4-yl)oxy)piperidine-1-carboxylate. To a solution of tert-butyl 4-((1-(4-amino-2-fluorophenyl)piperidin-4-yl)oxy)piperidine-1-carboxylate (11.0 g, 28.0 mmol, 1.00 eq) and 3-bromopiperidine-2,6-dione (13.5 g, 70.1 mmol, 2.50 eq) in DMF (200 mL) was added DIEA (10.9 g, 84.1 mmol, 14.7 mL, 3.00 eq) at 25 °C. The mixture was stirred at 80 °C for 12 h. LCMS showed tert-butyl 4-((1-(4-amino-2-fluorophenyl)piperidin-4-yl)oxy)piperidine-1-carboxylate remained little and the desired mass was detected. Water (100 mL) was added to the mixture and extracted with EtOAc (50 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=2:1 to 2:3) to give the title compound (4.10 g, 5.52 mmol, 19.7% yield, 67.9% purity) as a green solid. LCMS (ES+): m/z 505.3 [M+H]⁺.

Synthesis of 3-((3-fluoro-4-(4-(piperidin-4-yloxy)piperidin-1-yl)phenyl)amino)piperidine-2,6-dione. To a solution of tert-butyl 4-((1-(4-((2,6-dioxopiperidin-3-yl)amino)-2-fluorophenyl)piperidin-4-yl)oxy)piperidine-1-carboxylate (1.00 g, 1.98 mmol, 1.00 eq) in DCM (7.50 mL) was added TFA (3.84 g, 33.7 mmol, 2.50 mL, 17.0 eq) at 25 °C. The mixture was stirred at 25 °C for 0.5 h. LCMS showed tert-butyl 4-((1-(4-((2,6-dioxopiperidin-3-yl)amino)-2-fluorophenyl)piperidin-4-yl)oxy)piperidine-1-carboxylate was consumed completely and the desired mass was detected. The reaction mixture was concentrated under reduced pressure to give the title compound (1.00 g, crude, TFA) as a brown oil. LCMS (ES+): m/z 405.2 [M+H]⁺.

Synthesis of trans-3-((3-fluoro-4-(4-((1-(4-((5-fluoro-4-(3-(2-oxo-1,2-dihydropyridin-3-yl)phenyl)pyrimidin-2-yl)amino)cyclohexane-1-carbonyl)piperidin-4-yl)oxy)piperidin-1-yl)phenyl)amino)piperidine-2,6-dione (Compound 403). To a solution of trans-4-[[5-fluoro-4-[3-(2-oxo-1H-pyridin-3-yl)phenyl]pyrimidin-2-yl]amino]cyclohexanecarboxylic acid (394 mg, 964 μmol, 0.500 eq) in DMF (10 mL) were added DIEA (1.50 g, 11.6 mmol, 2.02 mL, 6.00 eq) and HATU (257 mg, 675 μmol, 0.350 eq) at 25 °C, then 3-((3-fluoro-4-(4-(piperidin-4-yloxy)piperidin-1-yl)phenyl)amino)piperidine-2,6-dione (1.00 g, 1.93 mmol, 1.00 eq, TFA) was added to the reaction mixture at 25 °C. The mixture was stirred at 25 °C for 1 h. LCMS showed 3-((3-fluoro-4-(4-(piperidin-4-yloxy)piperidin-1-yl)phenyl)amino)piperidine-2,6-dione was consumed and the desired mass was detected. The reaction mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to give the title compound (Compound 403) (113 mg, 133 μmol, 6.87% yield, 93.1% purity) as a light yellow solid. LCMS (ES⁺): m/z 795.4 [M+H]⁺.

### Example 416. Synthesis of trans-3-((3-fluoro-4-(4-((1-(4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperazin-1-yl)phenyl)amino)piperidine-2,6-dione (Compound 376)

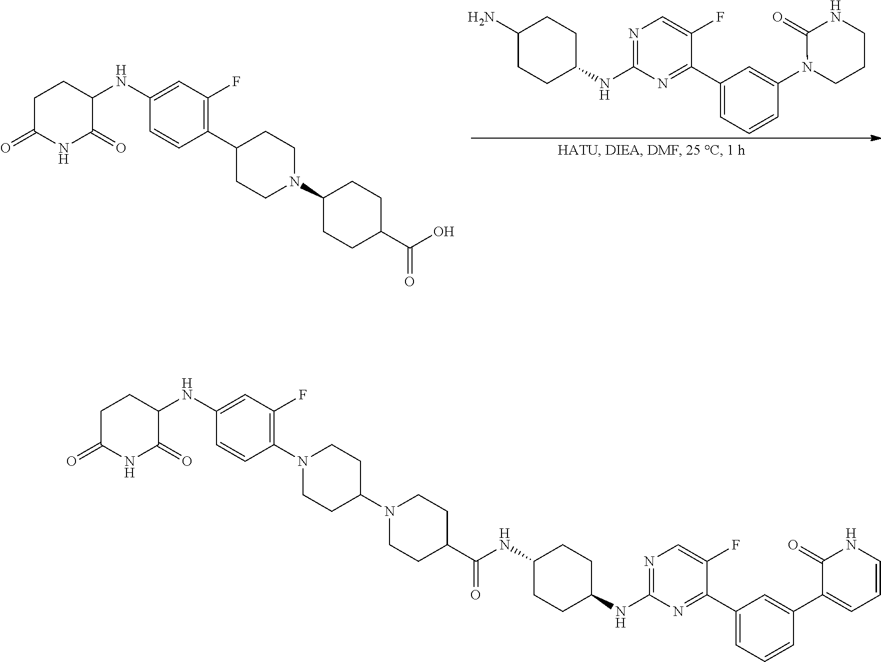

Synthesis of trans-3-((3-fluoro-4-(4-((1-(4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperazin-1-yl)phenyl)amino)piperidine-2,6-dione (Compound 376). To a solution of trans-4-[[5-fluoro-4-[3-(2-oxo-1H-pyridin-3-yl)phenyl]pyrimidin-2-yl]amino]cyclohexanecarboxylic acid (430 mg, 966 μmol, 1.00 eq, HCl) in DMF (5 mL) were added NMI (476 mg, 5.80 mmol, 462 μL, 6.00 eq) and TCFH (596 mg, 2.13 mmol, 2.20 eq) at 25 °C. At last, 3-((3-fluoro-4-(4-(piperidin-4-ylmethyl)piperazin-1-yl)phenyl)amino)piperidine-2,6-dione (500 mg, 966 μmol, 1.00 eq, TFA) was added at 25 °C. The mixture was stirred at 25 °C for 1 h. LCMS showed trans-4-[[5-fluoro-4-[3-(2-oxo-1H-pyridin-3-yl)phenyl]pyrimidin-2-yl]amino]cyclohexanecarboxylic acid was remaining and the desired mass was detected. The mixture was stirred at 40 °C for 10 h. LCMS showed trans-4-[[5-fluoro-4-[3-(2-oxo-1H-pyridin-3-yl)phenyl]pyrimidin-2-yl]amino]cyclohexanecarboxylic acid was consumed and the desired mass was detected. The mixture was allowed to room temperature and filtered. The reaction mixture was filtered and the filtrate was purified by prep-HPLC (column: Phenomenex Gemini-NX 80*40 mm*3 um; mobile phase: [H₂O (10 mM NH₄HCO₃)-ACN]; gradient: 25%-60% B over 8.0 min) to give the title compound (Compound 376) (124 mg, 149 μmol, 15.4% yield, 95.1% purity) as a white solid. LCMS (ES⁺): m/z 794.4 [M+H]⁺

### Example 417. Synthesis of trans-(1r,4R)—N-((1r,4R)-4-(4-(3-(2,6-dioxopiperidin-3-yl)-1-methyl-1H-indazol-6-yl)piperazin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 404)

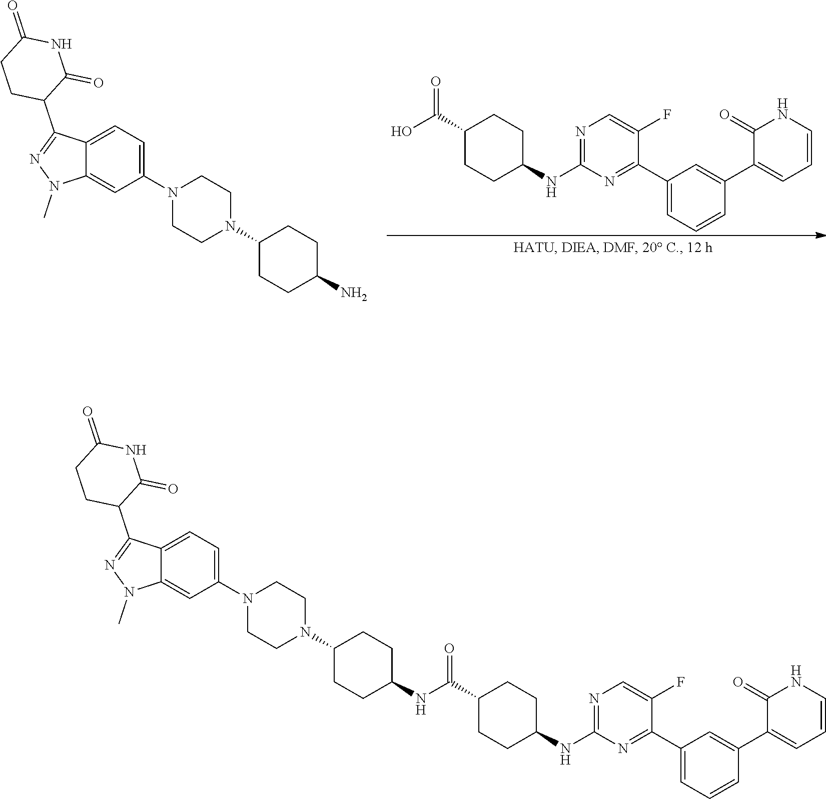 [SMILES: Cn1nc(C2CCC(=O)NC2=O)c2ccc(N3CCN([C@H]4CC[C@H](N)CC4)CC3)cc21 ; O=c1[nH]cccc1-c1cccc(-c2nc(N[C@H]3CC[C@H](C(=O)O)CC3)ncc2F)c1 ; Cn1nc(C2CCC(=O)NC2=O)c2ccc(N3CCN([C@H]4CC[C@H](NC(=O)[C@H]5CC[C@H](Nc6ncc(F)c(-c7cccc(-c8ccc[nH]c8=O)c7)n6)CC5)CC4)CC3)cc21]

Synthesis of trans-(1r,4R)—N-((1r,4R)-4-(4-(3-(2,6-dioxopiperidin-3-yl)-1-methyl-H-indazol-6-yl)piperazin-1-yl)cyclohexyl)-4-((5-fluoro-4-(3-(2-oxo-1,2-dihydropyridin-3-yl)phenyl)pyrimidin-2-yl)amino)cyclohexane-1-carboxamide (Compound 404). To a solution of trans-4-((5-fluoro-4-(3-(2-oxo-1,2-dihydropyridin-3-yl)phenyl)pyrimidin-2-yl)amino)cyclohexane-1-carboxylic acid (175 mg, 429 μmol, 0.500 eq) in DMF (5 mL) were added DIEA (555 mg, 4.30 mmol, 748 μL, 5.00 eq) and HATU (114 mg, 300 μmol, 0.350 eq) at 20 °C. Then To the mixture was added trans-3-(6-(4-(4-aminocyclohexyl)piperazin-1-yl)-1-methyl-1H-indazol-3-yl)piperidine-2,6-dione (462 mg, 858 μmol, 1.00 eq, TFA) at 20 °C. The mixture was stirred at 20 °C for 12 h. LCMS showed trans-3-(6-(4-(4-aminocyclohexyl)piperazin-1-yl)-1-methyl-1H-indazol-3-yl)piperidine-2,6-dione was consumed and the desired mass was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 20%-55% B over 8.0 min) to give the title compound (Compound 404) (38.1 mg, 44.9 μmol, 5.23% yield, 95.9% purity) as a white solid. LCMS (ES⁺): m/z 815.4 [M+H]⁺

### Example 418. Synthesis of trans-2-(4-((4-(4-((2,6-dioxopiperidin-3-yl)amino)-2-fluorophenyl)piperidin-1-yl)methyl)piperidin-1-yl)-N-(4-((5-fluoro-4-(3-(2-oxo-1,2-dihydropyridin-3-yl)phenyl)pyrimidin-2-yl)amino)cyclohexyl)acetamide (Compound 365)

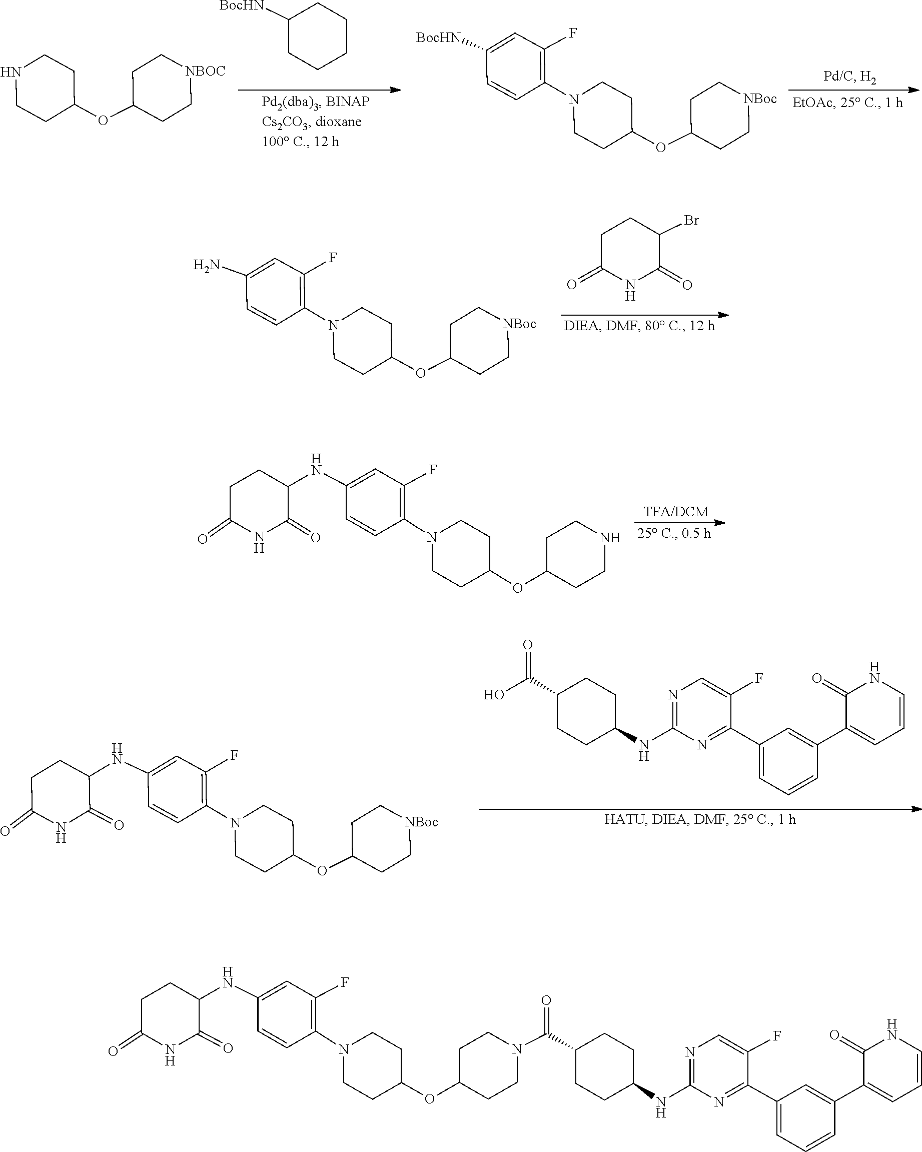

Synthesis of trans-tert-butyl (4-((5-fluoro-4-(3-(2-methoxypyridin-3-yl)phenyl)pyrimidin-2-yl)amino)cyclohexyl)carbamate. To a solution of trans-tert-butyl (4-aminocyclohexyl)carbamate (679 mg, 3.17 mmol, 1.00 eq) and 2-chloro-5-fluoro-4-(3-(2-methoxypyridin-3-yl)phenyl)pyrimidine (1.00 g, 3.17 mmol, 1.00 eq) in dioxane (50 mL) were added BINAP (177 mg, 285 μmol, 0.0900 eq), Pd₂(dba)₃ (145 mg, 158 μmol, 0.0500 eq) and Cs₂CO₃ (3.10 g, 9.50 mmol, 3.00 eq) at 20 °C under N₂, the mixture was stirred at 100 °C for 12 h under N₂. LCMS showed 2-chloro-5-fluoro-4-(3-(2-methoxypyridin-3-yl)phenyl)pyrimidine was consumed and the desired target MS was detected. H₂O (50 mL) was added to the mixture and the mixture was extracted with EtOAc (30 mL*3), the combined organic layers were washed with brine (50 mL*3), dried over Na₂SO₄, filtered and the filtrate was concentrated under reduced pressure to give a residue. The residue was purified by MPLC (SiO₂, PE/EtOAc=1/0 to 0/1) to give the title compound (800 mg, 1.62 mmol, 51.2% yield) as a yellow solid. LCMS (ES⁺): m/z 494.3 [M+H]⁺

Synthesis of trans-3-(3-(2-((4-aminocyclohexyl)amino)-5-fluoropyrimidin-4-yl)phenyl)pyridin-2(1H)-one. A solution of trans-tert-butyl ((4-((5-fluoro-4-(3-(2-methoxypyridin-3-yl)phenyl)pyrimidin-2-yl)amino)cyclohexyl)carbamate (800 mg, 1.62 mmol, 1.00 eq) in HCl/dioxane (15 mL) was stirred at 80 °C for 12 h. LCMS showed trans-tert-butyl ((-4-((5-fluoro-4-(3-(2-methoxypyridin-3-yl)phenyl)pyrimidin-2-yl)amino)cyclohexyl)carbamate was consumed and the desired target MS was detected. The mixture was concentrated under reduced pressure to give the title compound (750 mg, crude, HCl) as a yellow solid. LCMS (ES⁺): m/z 380.2 [M+H]⁺

Synthesis of trans-2-bromo-N-(4-((5-fluoro-4-(3-(2-oxo-1,2-dihydropyridin-3-yl)phenyl)pyrimidin-2-yl)amino)cyclohexyl)acetamide. To a solution of trans-tert-butyl ((4-((5-fluoro-4-(3-(2-methoxypyridin-3-yl)phenyl)pyrimidin-2-yl)amino)cyclohexyl)carbamate (375 mg, 902 μmol, 1.00 eq, HCl) in DCM (5 mL) were added DIEA (349 mg, 2.71 mmol, 471 μL, 3.00 eq) and 2-bromoacetyl bromide (182 mg, 902 μmol, 78.6 μL, 1.00 eq) at 0 °C. The mixture was stirred at 0 °C for 50 min. LCMS showed trans-tert-butyl ((-4-((5-fluoro-4-(3-(2-methoxypyridin-3-yl)phenyl)pyrimidin-2-yl)amino)cyclohexyl)carbamate was consumed completely and the desired target MS was detected. The mixture was concentrated under reduced pressure to give the title compound (750 mg, crude) as a brown oil. LCMS (ES⁺): m/z 500.2 [M+H]⁺

Synthesis of trans-2-(4-((4-(4-((2,6-dioxopiperidin-3-yl)amino)-2-fluorophenyl)piperidin-1-yl)methyl)piperidin-1-yl)-N-(4-((5-fluoro-4-(3-(2-oxo-1,2-dihydropyridin-3-yl)phenyl)pyrimidin-2-yl)amino)cyclohexyl)acetamide (Compound 365). To a solution of 3-((3-fluoro-4-(1-(piperidin-4-ylmethyl)piperidin-4-yl)phenyl)amino)piperidine-2,6-dione (698 mg, 1.35 mmol, 1.50 eq, TFA) and trans-2-bromo-N—(-4-((5-fluoro-4-(3-(2-oxo-1,2-dihydropyridin-3-yl)phenyl)pyrimidin-2-yl)amino)cyclohexyl)acetamide (451 mg, 901 μmol, 1.00 eq) in DMF (10 mL) was added DIEA (348 mg, 2.70 mmol, 468 μL, 3.00 eq) at 20 °C, the mixture was stirred at 20 °C for 2 h. LCMS showed trans-2-bromo-N—(-4-((5-fluoro-4-(3-(2-oxo-1,2-dihydropyridin-3-yl)phenyl)pyrimidin-2-yl)amino)cyclohexyl)acetamide remained and the desired target MS was detected. Then the mixture was stirred at 40 °C for another 2 h. LCMS showed trans-2-bromo-N—(-4-((5-fluoro-4-(3-(2-oxo-1,2-dihydropyridin-3-yl)phenyl)pyrimidin-2-yl)amino)cyclohexyl)acetamide was consumed and the desired target MS was detected. The mixture was concentrated under reduced pressure to give a residue. The residue was purified by prep-HPLC (column: Waters Xbridge Prep OBD C18 150*40 mm*Oum; mobile phase: [H₂O (10 mM NH₄HCO₃)-ACN]; gradient: 35%-65% B over 8.0 min) to give the title compound (Compound 365) (53.0 mg, 61.3 μmol, 8.08% yield, 95.1% purity) as a white solid. LCMS (ES⁺): m/z 822.4 [M+H]⁺

### Example 419. Synthesis of trans-1′-(4-((2,6-dioxopiperidin-3-yl)amino)-2-fluorophenyl)-N-(4-((5-fluoro-4-(3-(2-oxo-1,2-dihydropyridin-3-yl)phenyl)pyrimidin-2-yl)amino)cyclohexyl)-[1,4′-bipiperidine]-4-carboxamide (Compound 429)

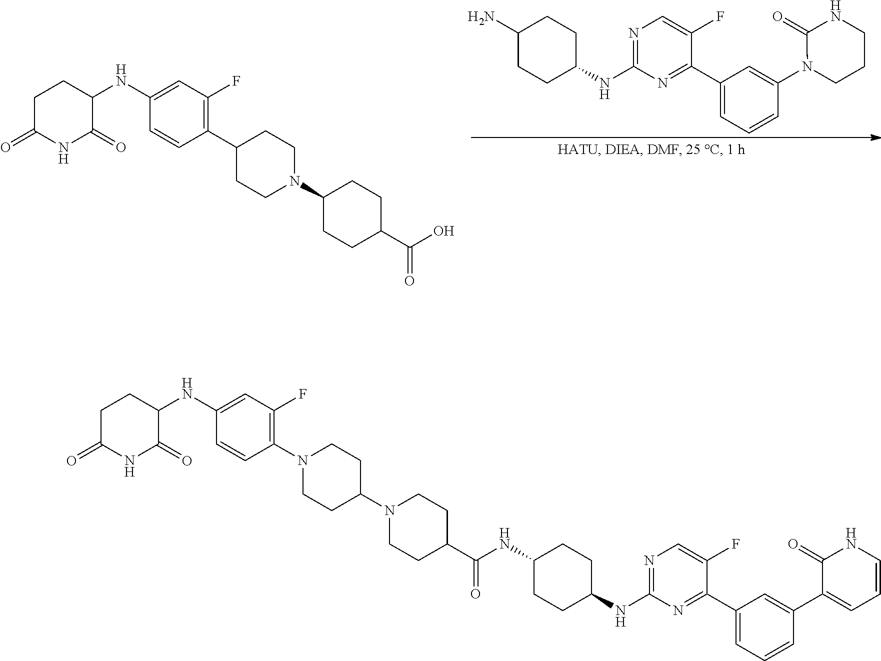

Synthesis of trans-1′-(4-((2,6-dioxopiperidin-3-yl)amino)-2-fluorophenyl)-N-(4-((5-fluoro-4-(3-(2-oxo-1,2-dihydropyridin-3-yl)phenyl)pyrimidin-2-yl)amino)cyclohexyl)-[1,4′-bipiperidine]-4-carboxamide (Compound 429). To a solution of 1-[1-[4-[(2,6-dioxo-3-piperidyl)amino]-2-fluoro-phenyl]-4-piperidyl]piperidine-4-carboxylic acid (370 mg, 677 μmol, 1.00 eq, TFA) in DMF (1 mL) was added DIEA (87.5 mg, 677 μmol, 117 μL, 1.00 eq) until pH~7. To a solution of trans-3-(3-(2-((-4-aminocyclohexyl)amino)-5-fluoropyrimidin-4-yl)phenyl)pyridin-2(1H)-one (225 mg, 541 μmol, 0.800 eq, HCl) in DMF (2 mL) was added DIEA (87.5 mg, 677 μmol, 117 μL, 1.00 eq) until pH~7. Then T₄P (243 mg, 677 μmol, 50%, 1.00 eq) was added to the mixture of the two solutions. The mixture was stirred at 25 °C for 12 h. LC-MS showed the desired mass was detected. The reaction mixture was filtered. The residue was purified by prep-HPLC (column: 3_Phenomenex Luna C18 75*30 mm*3 um; mobile phase: [H₂O (0.1% TFA)-ACN]; gradient: 12%-32% B over 9.0 min) to give the title compound (Compound 429) (96.9 mg, 113 μmol, 16.7% yield, 92.9% purity) as a white solid. LCMS (ES⁺): m/z 794.3 [M+H]⁺.

### Example 420. Synthesis of trans-1′-(4-(((R)-2,6-dioxopiperidin-3-yl)amino)-2-fluorophenyl)-N-((1r,4r)-4-((5-fluoro-4-(3-(2-oxo-1,2-dihydropyridin-3-yl)phenyl)pyrimidin-2-yl)amino)cyclohexyl)-[1,4′-bipiperidine]-4-carboxamide (Compound 577) and trans-1′-(4-(((S)-2,6-dioxopiperidin-3-yl)amino)-2-fluorophenyl)-N-((1r,4r)-4-((5-fluoro-4-(3-(2-oxo-1,2-dihydropyridin-3-yl)phenyl)pyrimidin-2-yl)amino)cyclohexyl)-[1,4′-bipiperidine]-4-carboxamide (Compound 702) (The Two Isomers were Assigned Arbitrarily)

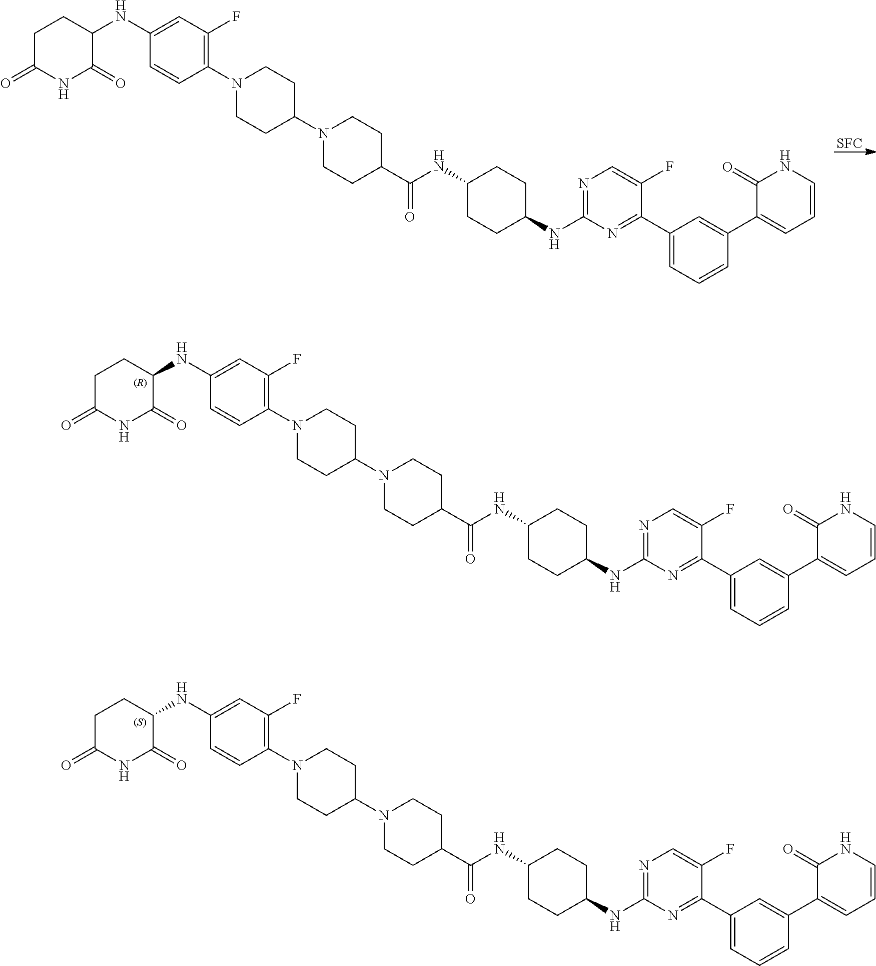

Synthesis of trans-1′-(4-(((R)-2,6-dioxopiperidin-3-yl)amino)-2-fluorophenyl)-N-((1r,4r)-4-((5-fluoro-4-(3-(2-oxo-1,2-dihydropyridin-3-yl)phenyl)pyrimidin-2-yl)amino)cyclohexyl)-[1,4′-bipiperidine]-4-carboxamide (Compound 577) and 1′-(4-(((S)-2,6-dioxopiperidin-3-yl)amino)-2-fluorophenyl)-N-((1r,4r)-4-((5-fluoro-4-(3-(2-oxo-1,2-dihydropyridin-3-yl)phenyl)pyrimidin-2-yl)amino)cyclohexyl)-[1,4′-bipiperidine]-4-carboxamide (Compound 702). trans-1′-(4-((2,6-dioxopiperidin-3-yl)amino)-2-fluorophenyl)-N-(4-((5-fluoro-4-(3-(2-oxo-1,2-dihydropyridin-3-yl)phenyl)pyrimidin-2-yl)amino)cyclohexyl)-[1,4′-bipiperidine]-4-carboxamide (Compound 429) was separated by SFC (column: ChiralPak IH, 250*30 mm, 10 um; mobile phase: [CO₂-IPA:CAN=1:1]; B %: 60%, isocratic elution mode) to give the title compound (Compound 577) (17.1 mg, 20.6 mol, 17.8% yield, 95.9% purity) as a brown solid. LCMS (ES⁺): m/z 794.4 [M+H]+ and the title compound (Compound 702) as a pale yellow solid. LCMS (ES⁺): m/z 794.4 [M+H]⁺

### Example 421. Synthesis of trans-4-(4-(4-((2,6-dioxopiperidin-3-yl)amino)-2-fluorophenyl)piperidin-1-yl)-N-(trans-4-((5-fluoro-4-(3-(2-oxo-1,2-dihydropyridin-3-yl)phenyl)pyrimidin-2-yl)amino)cyclohexyl)cyclohexane-1-carboxamide (Compound 396)

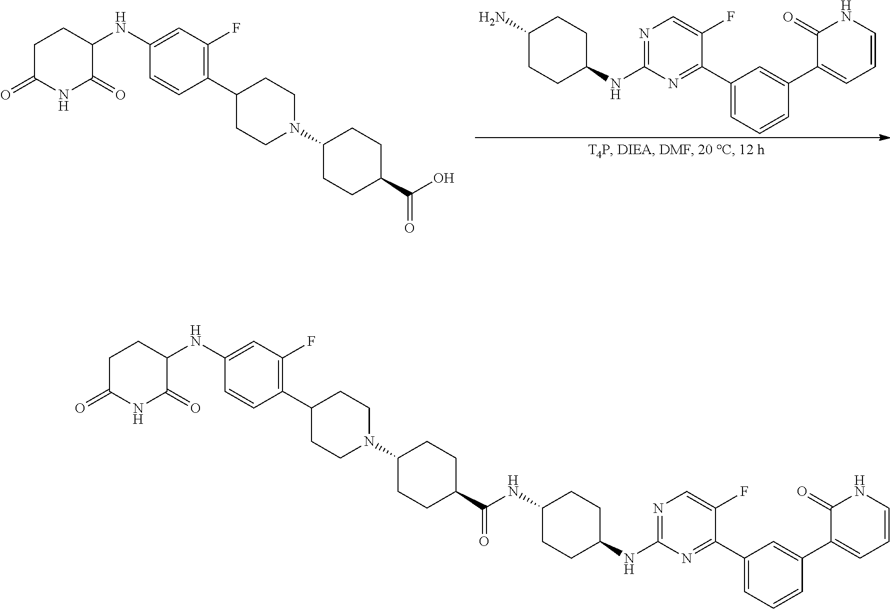 [SMILES: O=C1CCC(Nc2ccc(C3CCN([C@H]4CC[C@H](C(=O)O)CC4)CC3)c(F)c2)C(=O)N1 ; N[C@H]1CC[C@H](Nc2ncc(F)c(-c3cccc(-c4ccc[nH]c4=O)c3)n2)CC1 ; O=C1CCC(Nc2ccc(C3CCN([C@H]4CC[C@H](C(=O)N[C@H]5CC[C@H](Nc6ncc(F)c(-c7cccc(-c8ccc[nH]c8=O)c7)n6)CC5)CC4)CC3)c(F)c2)C(=O)N1]

Synthesis of trans-4-(4-(4-((2,6-dioxopiperidin-3-yl)amino)-2-fluorophenyl)piperidin-1-yl)-N-(trans-4-((5-fluoro-4-(3-(2-oxo-1,2-dihydropyridin-3-yl)phenyl)pyrimidin-2-yl)amino)cyclohexyl)cyclohexane-1-carboxamide (Compound 396). To a solution of trans-4-(4-(4-((2,6-dioxopiperidin-3-yl)amino)-2-fluorophenyl)piperidin-1-yl)cyclohexane-1-carboxylic acid (400 mg, 927 μmol, 1.00 eq) in DMF (4 mL) was added 3-(3-(2-((trans-4-aminocyclohexyl)amino)-5-fluoropyrimidin-4-yl)phenyl)pyridin-2(1H)-one (351 mg, 927 μmol, 1.00 eq), DIEA (239 mg, 1.85 mmol, 322 μL, 2.00 eq) and T₄P (667 mg, 1.85 mmol, 2.00 eq) at 20 °C. The mixture was stirred at 20 °C for 12 h. LC-MS showed the desired mass was detected. The reaction mixture was filtered and the filtrate concentrated under high vacuum. The residue was purified by prep-HPLC (Phenomenex luna C18 100*40 mm*3 um; mobile phase: [H₂O (0.2% FA)-ACN]; gradient: 5%-40% B over 8.0 min) to give the title compound (Compound 396) (129 mg, 159 μmol, 21.7% yield, 99.1% purity, 0.30 FA) as a white solid. LCMS (ES⁺): m/z 793.4 [M+H]⁺.

### Example 422. Synthesis of trans-4-(4-(4-(((R)-2,6-dioxopiperidin-3-yl)amino)-2-fluorophenyl)piperidin-1-yl)-N-(trans-4-((5-fluoro-4-(3-(2-oxo-1,2-dihydropyridin-3-yl)phenyl)pyrimidin-2-yl)amino)cyclohexyl)cyclohexane-1-carboxamide (Compound 454)

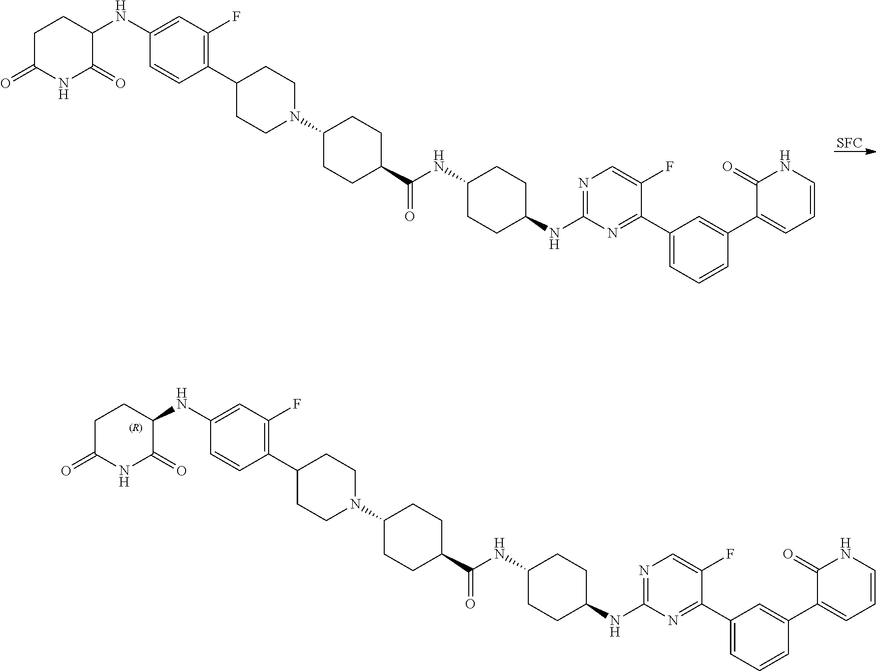

Synthesis of trans-4-(4-(4-(((R)-2,6-dioxopiperidin-3-yl)amino)-2-fluorophenyl)piperidin-1-yl)-N-(trans-4-((5-fluoro-4-(3-(2-oxo-1,2-dihydropyridin-3-yl)phenyl)pyrimidin-2-yl)amino)cyclohexyl)cyclohexane-1-carboxamide (Compound 454). The residue was purified by SFC (DAICEL CHIRALPAK IC (250 mm*30 mm, 10 um); mobile phase: [Heptane-IPA:ACN=4:1]; B %: 70%, isocratic elution mode) to give the title compound (Compound 454) (10.9 mg, 13.1 μmol, 13.0% yield, 95.4% purity) as a white solid. LCMS (ES⁺): m/z 793.4 [M+H]⁺.

### Example 423. Synthesis of trans-4-(4-(4-((2,6-dioxopiperidin-3-yl)amino)-2-fluorophenyl)piperazin-1-yl)-N-(trans-4-((5-fluoro-4-(3-(2-oxo-1,2-dihydropyridin-3-yl)phenyl)pyrimidin-2-yl)amino)cyclohexyl)cyclohexane-1-carboxamide (Compound 450)

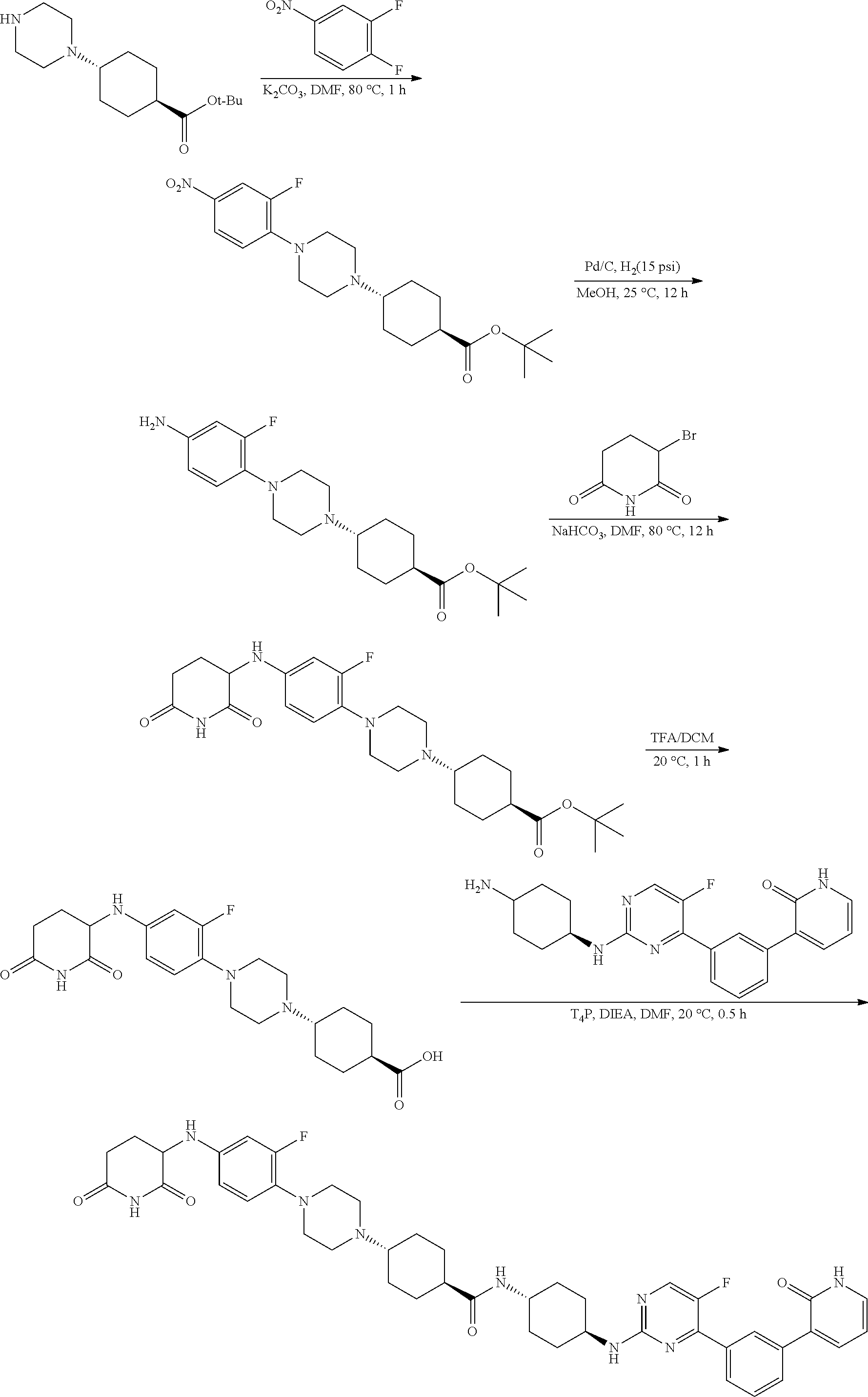

Synthesis of trans-tert-butyl-4-(4-(2-fluoro-4-nitrophenyl)piperazin-1-yl)cyclohexane-1-carboxylate. To a solution of tert-butyl trans-4-(piperazin-1-yl)cyclohexane-1-carboxylate (6.00 g, 22.3 mmol, 1.00 eq) and 1,2-difluoro-4-nitro-benzene (3.56 g, 22.3 mmol, 2.47 mL, 1.00 eq) in DMF (100 mL) was added K₂CO₃ (6.18 g, 44.7 mmol, 2.00 eq) at 20 °C. The mixture was stirred at 80 °C for 1 h. LC-MS showed the desired mass was detected. The reaction mixture was diluted with H₂O 300 mL and extracted with EtOAc 300 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (14.0 g, 30.9 mmol, 69.1% yield, 90.0% purity) as a yellow solid. LCMS (ES⁺): m/z 408.3 [M+H]⁺.

Synthesis of trans-tert-butyl-4-(4-(4-amino-2-fluorophenyl)piperazin-1-yl)cyclohexane-1-carboxylate. To a solution of trans-tert-butyl-4-(4-(2-fluoro-4-nitrophenyl)piperazin-1-yl)cyclohexane-1-carboxylate (13.0 g, 31.9 mmol, 1.00 eq) in MeOH (200 mL) was added 10% Pd/C (6.50 g, 30.5 mmol, 50.0% purity, 9.57^{e-1} eq) under N₂ atmosphere at 25 °C. The suspension was degassed and purged with H₂ for 3 times. The mixture was stirred under H₂ (15 Psi) at 25 °C for 12 h. LC-MS showed the desired mass was detected. The reaction mixture was filtered and concentrated under reduced pressure to give the title compound (12.0 g, crude) as a white solid. LCMS (ES⁺): m/z 378.2 [M+H]⁺.

Synthesis of trans-tert-butyl-4-(4-(4-((2,6-dioxopiperidin-3-yl)amino)-2-fluorophenyl)piperazin-1-yl)cyclohexane-1-carboxylate. The reaction was set up in two batches. To a solution of trans-tert-butyl-4-(4-(4-amino-2-fluorophenyl)piperazin-1-yl)cyclohexane-1-carboxylate (6.00 g, 15.8 mmol, 1.00 eq) 3-bromopiperidine-2,6-dione (9.16 g, 47.6 mmol, 3.00 eq) in DMF (100 mL) was added NaHCO₃ (2.67 g, 31.7 mmol, 1.24 mL, 2.00 eq) at 20 °C. The mixture was stirred at 80 °C for 12 h. LC-MS showed the desired mass was detected. Two batches in parallel were combined, the reaction mixture was quenched by addition H₂O 200 mL, and extracted with EtOAc 60 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (8.00 g, 14.2 mmol, 44.8% yield, 87.0% purity) as a white solid. LCMS (ES⁺): m/z 489.3 [M+H]⁺.

Synthesis of trans-4-(4-(4-((2,6-dioxopiperidin-3-yl)amino)-2-fluorophenyl)piperazin-1-yl)cyclohexane-1-carboxylic acid. To a solution of trans-tert-butyl-4-(4-(4-((2,6-dioxopiperidin-3-yl)amino)-2-fluorophenyl)piperazin-1-yl)cyclohexane-1-carboxylate (1.50 g, 3.07 mmol, 1.00 eq) in DCM (20 mL) was added TFA (12.2 g, 107 mmol, 8 mL, 35.08 eq) at 20 °C. The mixture was stirred at 20 °C for 1 h. LC-MS showed the desired mass was detected. The reaction mixture was concentrated under reduced pressure to give the title compound (2.00 g, crude, TFA) as a black oil. LCMS (ES⁺): m/z 433.2 [M+H]⁺.

Synthesis of trans-4-(4-(4-((2,6-dioxopiperidin-3-yl)amino)-2-fluorophenyl)piperazin-1-yl)-N-(trans-4-((5-fluoro-4-(3-(2-oxo-1,2-dihydropyridin-3-yl)phenyl)pyrimidin-2-yl)amino)cyclohexyl)cyclohexane-1-carboxamide (Compound 450). To a solution of trans-4-(4-(4-((2,6-dioxopiperidin-3-yl)amino)-2-fluorophenyl)piperazin-1-yl)cyclohexane-1-carboxylic acid (1.00 g, 1.83 mmol, 1.00 eq, TFA) and 3-(3-(2-((trans-4-aminocyclohexyl)amino)-5-fluoropyrimidin-4-yl)phenyl)pyridin-2(1H)-one (833 mg, 2.20 mmol, 1.20 eq) in DMF (10 mL) was added DIEA (709 mg, 5.49 mmol, 956 μL, 3.00 eq) and T₄P (2.64 g, 3.66 mmol, 50.0% purity, 2.00 eq) at 20 °C. The mixture was stirred at 20 °C for 0.5 h. LC-MS showed the desired mass was detected. The reaction mixture was concentrated under reduced pressure. The residue was purified by prep-HPLC (column: Phenomenex luna C18 (250*70 mm, 15 um); mobile phase: [H₂O (0.2% FA)-ACN]; gradient: 20%-50% B over 20.0 min) to give the title compound (Compound 450) (506 mg, 623 μmol, 34.1% yield, 99.8% purity, 0.38 FA) as an off-white solid. LCMS (ES⁺): m/z 794.4 [M+H]⁺.

### Example 424. Synthesis of trans-4-(4-(4-(((R)-2,6-dioxopiperidin-3-yl)amino)-2-fluorophenyl)piperazin-1-yl)-N-((trans)-4-((5-fluoro-4-(3-(2-oxo-1,2-dihydropyridin-3-yl)phenyl)pyrimidin-2-yl)amino)cyclohexyl)cyclohexane-1-carboxamide (Compound 544) and trans-4-(4-(4-(((S)-2,6-dioxopiperidin-3-yl)amino)-2-fluorophenyl)piperazin-1-yl)-N-((trans)-4-((5-fluoro-4-(3-(2-oxo-1,2-dihydropyridin-3-yl)phenyl)pyrimidin-2-yl)amino)cyclohexyl)cyclohexane-1-carboxamide (Compound 517)

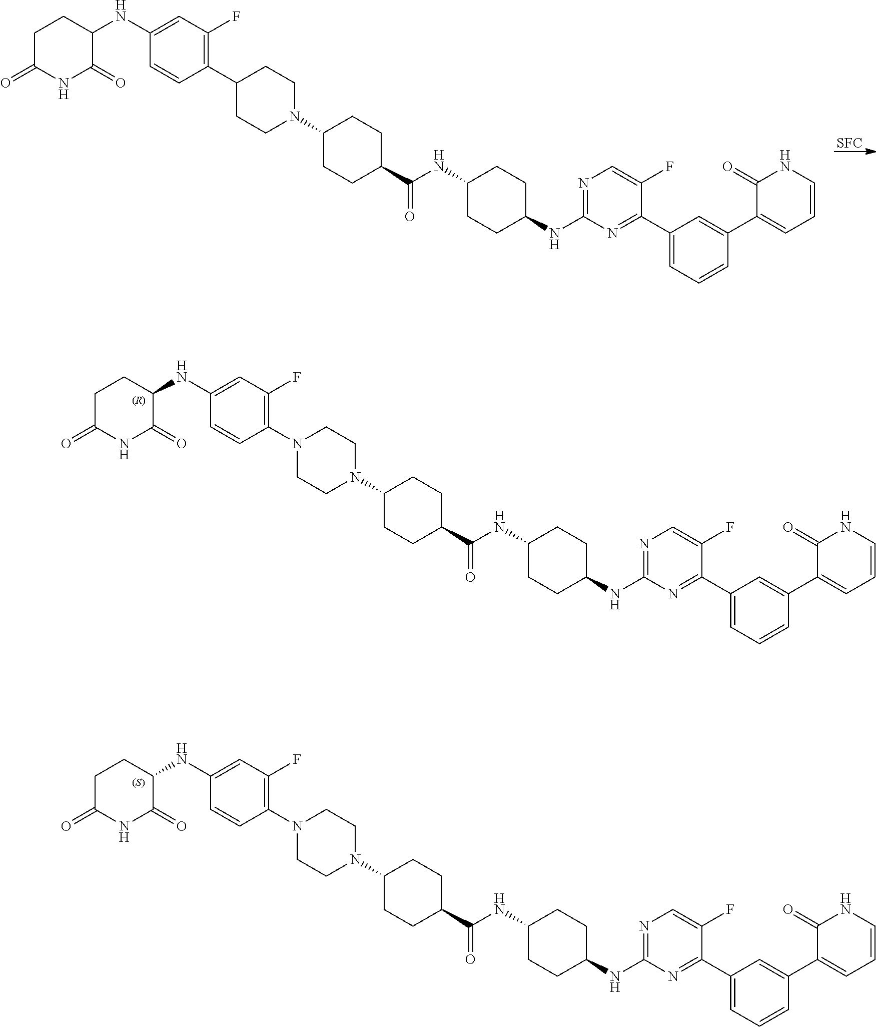

Synthesis of trans-4-(4-(4-(((R)-2,6-dioxopiperidin-3-yl)amino)-2-fluorophenyl)piperazin-1-yl)-N-((trans)-4-((5-fluoro-4-(3-(2-oxo-1,2-dihydropyridin-3-yl)phenyl)pyrimidin-2-yl)amino)cyclohexyl)cyclohexane-1-carboxamide (Compound 544) and trans-4-(4-(4-(((S)-2,6-dioxopiperidin-3-yl)amino)-2-fluorophenyl)piperazin-1-yl)-N-((trans)-4-((5-fluoro-4-(3-(2-oxo-1,2-dihydropyridin-3-yl)phenyl)pyrimidin-2-yl)amino)cyclohexyl)cyclohexane-1-carboxamide. The compound trans-4-(4-(4-((2,6-dioxopiperidin-3-yl)amino)-2-fluorophenyl)piperidin-1-yl)-N-(trans-4-((5-fluoro-4-(3-(2-oxo-1,2-dihydropyridin-3-yl)phenyl)pyrimidin-2-yl)amino)cyclohexyl)cyclohexane-1-carboxamide (Compound 450) (500 mg, 629 μmol, 1.00 eq) was further separated by SFC (SFC: column: ChiralPak IH, 250*30 mm, 10 um; mobile phase: [CO₂-IPA (0.1% NH₃H₂O)]; B %: 65%, isocratic elution mode. to give the title compound (Compound 544) (134.2 mg, 153 μmol, 24.4% yield, 90.9% purity) as a pale yellow solid LCMS (ES⁺): m/z 794.4 [M+H]+ and (Compound 517) (134.2 mg, 153 μmol, 24.4% yield, 90.9% purity) as a pale yellow solid. LCMS (ES⁺): m/z 794.4 [M+H]⁺.

### Example 425. Synthesis of 1′-(4-((2,6-dioxopiperidin-3-yl)amino)-2-fluorophenyl)-3′,3′-difluoro-N-(trans-4-((5-fluoro-4-(3-(2-oxo-1,2-dihydropyridin-3-yl)phenyl)pyrimidin-2-yl)amino)cyclohexyl)-[1,4′-bipiperidine]-4-carboxamide (Compound 599)

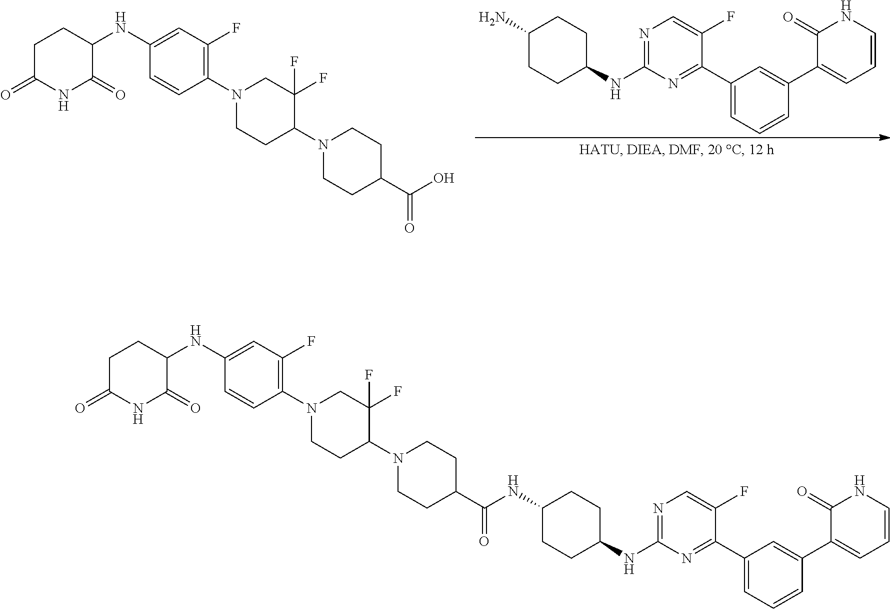 [SMILES: O=C1CCC(Nc2ccc(N3CCC(N4CCC(C(=O)O)CC4)C(F)(F)C3)c(F)c2)C(=O)N1 ; N[C@H]1CC[C@H](Nc2ncc(F)c(-c3cccc(-c4ccc[nH]c4=O)c3)n2)CC1 ; O=C1CCC(Nc2ccc(N3CCC(N4CCC(C(=O)N[C@H]5CC[C@H](Nc6ncc(F)c(-c7cccc(-c8ccc[nH]c8=O)c7)n6)CC5)CC4)C(F)(F)C3)c(F)c2)C(=O)N1]

Synthesis of 1′-(4-((2,6-dioxopiperidin-3-yl)amino)-2-fluorophenyl)-3′,3′-difluoro-N-(trans-4-((5-fluoro-4-(3-(2-oxo-1,2-dihydropyridin-3-yl)phenyl)pyrimidin-2-yl)amino)cyclohexyl)-[1,4′-bipiperidine]-4-carboxamide (Compound 599). To a solution of 1′-(4-((2,6-dioxopiperidin-3-yl)amino)-2-fluorophenyl)-3′,3-difluoro-[1,4′-bipiperidine]-4-carboxylic acid (500 mg, 1.07 mmol, 1.00 eq, TFA) in DMF (5 mL) was added 3-(3-(2-((trans-4-aminocyclohexyl)amino)-5-fluoropyrimidin-4-yl)phenyl)pyridin-2(1H)-one (404 mg, 1.07 mmol, 1.00 eq), DIEA (275 mg, 2.13 mmol, 371 μL, 2.00 eq) and HATU (811 mg, 2.13 mmol, 2.00 eq) at 20 °C. The mixture was stirred at 20 °C for 12 h. LC-MS showed the desired mass was detected. The reaction mixture was filtered and the filtrate concentrated under high vacuum. The residue was purified by prep-HPLC (Phenomenex Luna C18 100*30 mm*3 um; mobile phase: [H₂O (0.2% FA)-ACN]; gradient: 10%-45% B over 8.0 min) to give the title compound (Compound 599) (200.1 mg, 238 μmol, 22.3% yield, 98.8% purity) as a brown solid. LCMS (ES⁺): m/z 830.4 [M+H]⁺.

### Example 426. Synthesis of trans-(1r,4R)-4-(4-(3-(2,6-dioxopiperidin-3-yl)-1-methyl-1H-indazol-6-yl)piperazin-1-yl)-N-((1r,4R)-4-((5-fluoro-4-(3-(2-oxo-1,2-dihydropyridin-3-yl)phenyl)pyrimidin-2-yl)amino)cyclohexyl)cyclohexane-1-carboxamide (Compound 389)

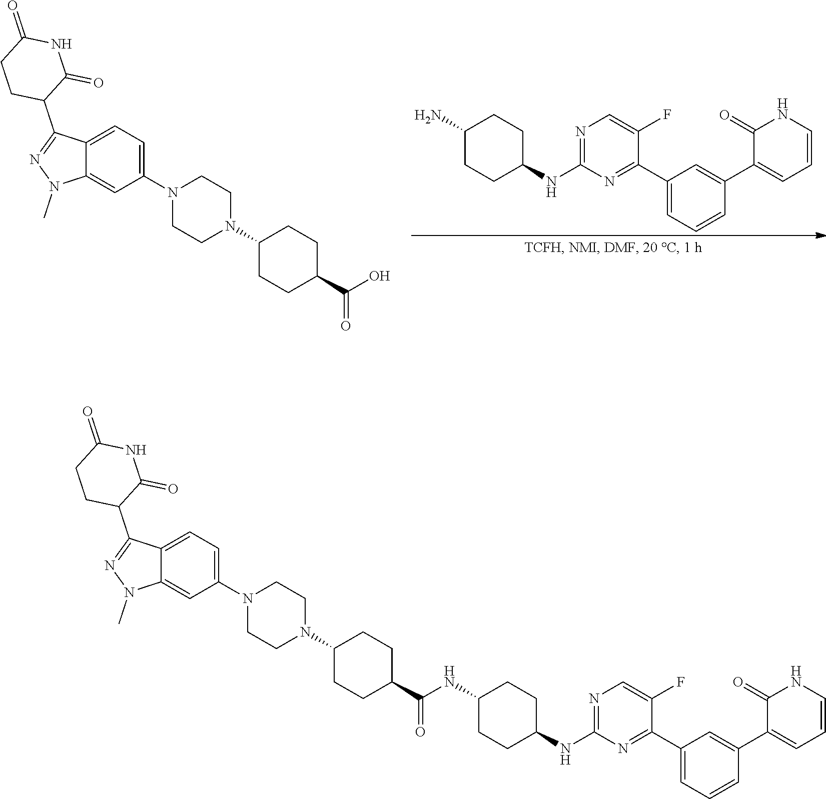 [SMILES: Cn1nc(C2CCC(=O)NC2=O)c2ccc(N3CCN([C@H]4CC[C@H](C(=O)O)CC4)CC3)cc21 ; N[C@H]1CC[C@H](Nc2ncc(F)c(-c3cccc(-c4ccc[nH]c4=O)c3)n2)CC1 ; Cn1nc(C2CCC(=O)NC2=O)c2ccc(N3CCN([C@H]4CC[C@H](C(=O)N[C@H]5CC[C@H](Nc6ncc(F)c(-c7cccc(-c8ccc[nH]c8=O)c7)n6)CC5)CC4)CC3)cc21]

Synthesis of trans-(1r,4R)-4-(4-(3-(2,6-dioxopiperidin-3-yl)-1-methyl-1H-indazol-6-yl)piperazin-1-yl)-N-((1r,4R)-4-((5-fluoro-4-(3-(2-oxo-1,2-dihydropyridin-3-yl)phenyl)pyrimidin-2-yl)amino)cyclohexyl)cyclohexane-1-carboxamide (Compound 389). To a solution of trans-4-(4-(3-(2,6-dioxopiperidin-3-yl)-1-methyl-1H-indazol-6-yl)piperazin-1-yl)cyclohexane-1-carboxylic acid (800 mg, 1.76 mmol, 1.00 eq) and trans-3-(3-(2-((4-aminocyclohexyl)amino)-5-fluoropyrimidin-4-yl)phenyl)pyridin-2(1H)-one (734 mg, 1.76 mmol, 1.00 eq, HCl) in DMF (15 mL) were added TCFH (396 mg, 1.41 mmol, 0.800 eq) and NMI (449 mg, 5.47 mmol, 436 μL, 3.10 eq) at 20 °C, the mixture was stirred at 20 °C for 1 h. LCMS showed trans-4-(4-(3-(2,6-dioxopiperidin-3-yl)-1-methyl-1H-indazol-6-yl)piperazin-1-yl)cyclohexane-1-carboxylic acid was consumed and the desired target MS was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 15%-45% B over 8.0 min) to give the title compound (Compound 389) (62.5 mg, 76.4 μmol, 5.42% yield, 99.6% purity) as a white solid. LCMS (ES⁺): m/z 815.4 [M+H]⁺

### Example 427. Synthesis of 3-(6-(1-(2-(1-((1R,3S)-3-((5-fluoro-4-(3-(5-methyl-2-oxopyridin-1(2H)-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 354)

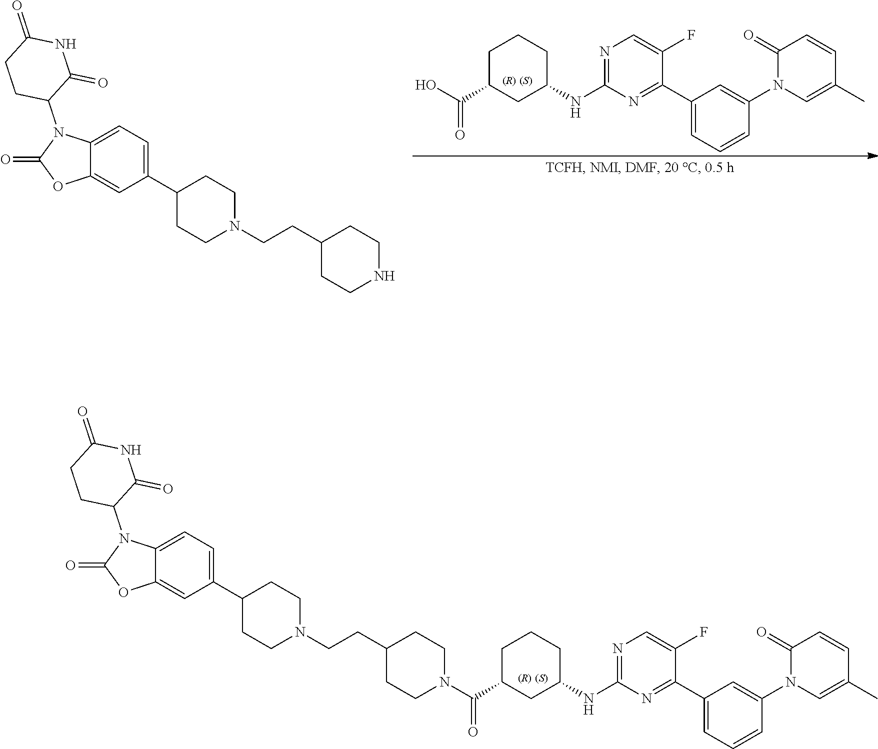

Synthesis of 3-(6-(1-(2-(1-((1R,3S)-3-((5-fluoro-4-(3-(5-methyl-2-oxopyridin-1(2H)-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 354). To a solution of (1R,3S)-3-((5-fluoro-4-(3-(5-methyl-2-oxopyridin-1(2H)-yl)phenyl)pyrimidin-2-yl)amino)cyclohexane-1-carboxylic acid (229 mg, 541 μmol, 1.00 eq) in DMF (3 mL) were added TCFH (182 mg, 649 μmol, 1.20 eq) and NMI (133 mg, 1.62 mmol, 129 μL, 3.00 eq) at 20 °C, then 3-(2-oxo-6-(1-(2-(piperidin-4-yl)ethyl)piperidin-4-yl)benzo[d]oxazol-3 (2H)-yl)piperidine-2,6-dione (300 mg, 541 μmol, 1.00 eq, TFA) was added to the mixture at 20 °C, the mixture was stirred at 20 °C for 0.5 h. LCMS showed 3-(2-oxo-6-(1-(2-(piperidin-4-yl)ethyl)piperidin-4-yl)benzo[d]oxazol-3 (2H)-yl)piperidine-2,6-dione was consumed and the desired target MS was detected. The mixture was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 25%-55% B over 8.0 min) to give the title compound (Compound 354) (20.0 mg, 21.8 μmol, 4.04% yield, 97.3% purity, FA) as a white solid. LCMS (ES⁺): m/z 845.4 [M+H]⁺

### Example 428. Synthesis of 3-(6-(1-(2-(1-((1R,3S)-3-((5-fluoro-4-(3-(2-oxo-1,3-oxazinan-3-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 355)

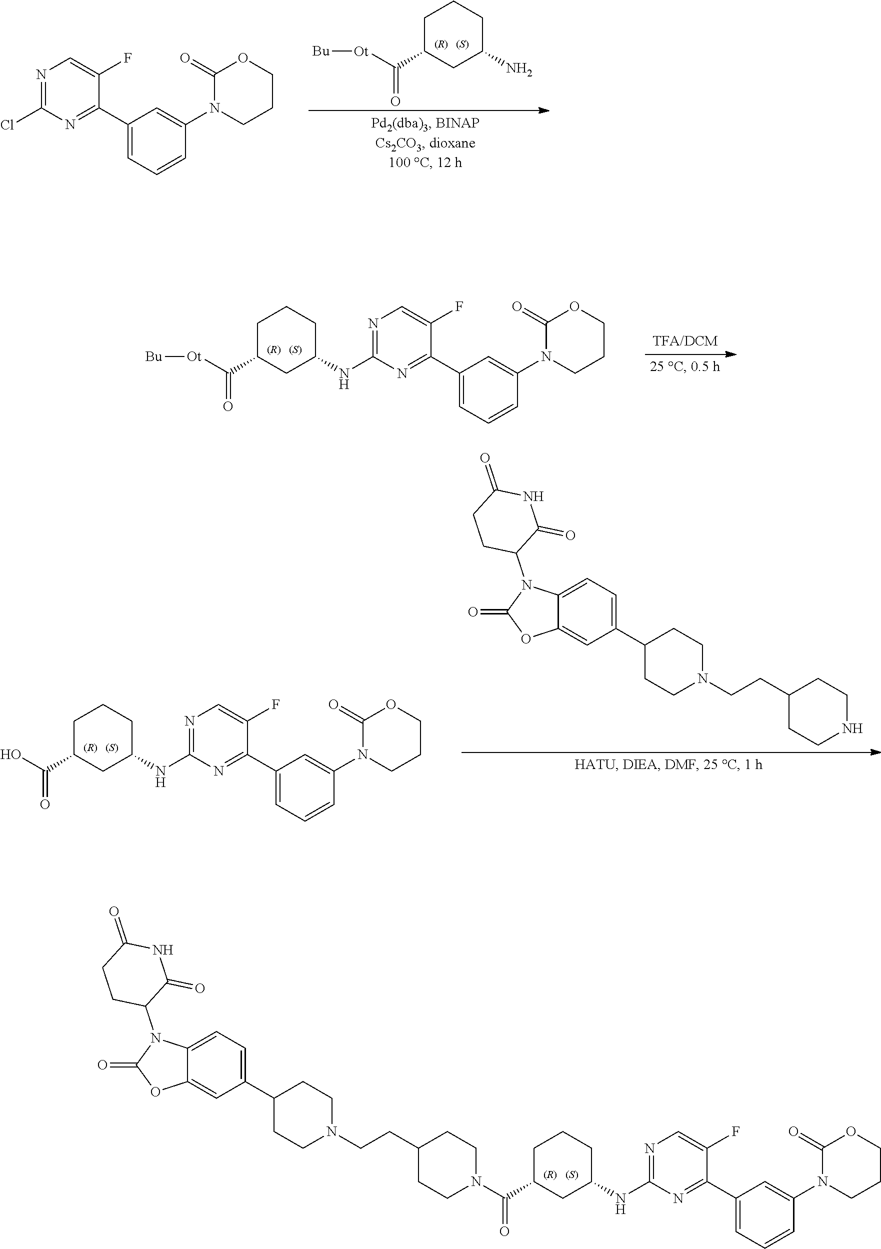

Synthesis of tert-butyl (1R,3S)-3-((5-fluoro-4-(3-(2-oxo-1,3-oxazinan-3-yl)phenyl)pyrimidin-2-yl)amino)cyclohexane-1-carboxylate. To a solution of 3-(3-(2-chloro-5-fluoropyrimidin-4-yl)phenyl)-1,3-oxazinan-2-one (500 mg, 1.62 mmol, 1.00 eq) and tert-butyl (1R,3S)-3-aminocyclohexane-1-carboxylate (389 mg, 1.95 mmol, 1.20 eq) in dioxane (20 mL) were added BINAP (60.7 mg, 97.5 μmol, 0.0600 eq), Cs₂CO₃ (1.06 g, 3.25 mmol, 2.00 eq) and Pd₂(dba)₃ (59.5 mg, 65.0 μmol, 0.0400 eq) at 25 °C under N₂, the mixture was stirred at 100 °C for 12 h under N₂. LCMS showed 3-(3-(2-chloro-5-fluoropyrimidin-4-yl)phenyl)-1,3-oxazinan-2-one was consumed completely and the desired mass was detected. The mixture was diluted with H₂O (20 mL) and then extracted with EtOAc (20 mL*3). The combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (562 mg, 1.19 mmol, 73.5% yield) as white solid. LCMS (ES⁺): m/z 471.2 [M+H]⁺

Synthesis of (1R,3S)-3-((5-fluoro-4-(3-(2-oxo-1,3-oxazinan-3-yl)phenyl)pyrimidin-2-yl)amino)cyclohexane-1-carboxylic acid. A solution of tert-butyl (1R,3S)-3-((5-fluoro-4-(3-(2-oxo-1,3-oxazinan-3-yl)phenyl)pyrimidin-2-yl)amino)cyclohexane-1-carboxylate (200 mg, 425 μmol, 1.00 eq) in DCM (3 mL) and TFA (1 mL) was stirred at 25 °C for 0.5 h. LCMS showed tert-butyl (1R,3S)-3-((5-fluoro-4-(3-(2-oxo-1,3-oxazinan-3-yl)phenyl)pyrimidin-2-yl)amino)cyclohexane-1-carboxylate was consumed completely and the desired mass was detected. The mixture was concentrated under reduced pressure to give the title compound (200 mg, crude) as a yellow oil. LCMS (ES⁺): m/z 415.3 [M+H]⁺

Synthesis of 3-(6-(1-(2-(1-((1R,3S)-3-((5-fluoro-4-(3-(2-oxo-1,3-oxazinan-3-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 355). To a solution of (1R,3S)-3-((5-fluoro-4-(3-(2-oxo-1,3-oxazinan-3-yl)phenyl)pyrimidin-2-yl)amino)cyclohexane-1-carboxylic acid (150 mg, 362 μmol, 1.00 eq) in DMF (5 mL) were added HATU (165 mg, 434 μmol, 1.20 eq) and DIEA (140 mg, 1.09 mmol, 189 μL, 3.00 eq) at 25 °C, then to the mixture was added 3-(2-oxo-6-(1-(2-(piperidin-4-yl)ethyl)piperidin-4-yl)benzo[d]oxazol-3 (2H)-yl)piperidine-2,6-dione (201 mg, 362 μmol, 1.00 eq, TFA) at 25 °C, the mixture was stirred at 25 °C for 1 h. LCMS showed (1R,3S)-3-((5-fluoro-4-(3-(2-oxo-1,3-oxazinan-3-yl)phenyl)pyrimidin-2-yl)amino)cyclohexane-1-carboxylic acid was consumed completely and the desired mass was detected. The mixture was diluted with H₂O (5 mL) and then extracted with EtOAc (5 mL*3). The combined organic layers were dried over Na₂SO₄, filtered and the filtrate was concentrated under reduced pressure to give a residue. The residue was purified by prep-HPLC (column: Phenomenex luna C18 100*40 mm*3 um; mobile phase: [H₂O (0.2% FA)-ACN]; gradient: 20%-55% B over 8.0 min) to give the title compound (Compound 355) (28.1 mg, 33.3 μmol, 9.19% yield, 99.1% purity) as a white solid. LCMS (ES⁺): m/z 837.4 [M+H]⁺

### Example 429. Synthesis of 3-(6-(1-((1-(2-(4-((5-fluoro-4-(3-(2-oxo-1,3-oxazinan-3-yl)phenyl)pyrimidin-2-yl)amino)piperidin-1-yl)-2-oxoethyl)piperidin-4-yl)methyl)piperidin-4-yl)-2-oxobenzo[d]oxazol-3 (2H)-yl)piperidine-2,6-dione (Compound 371)

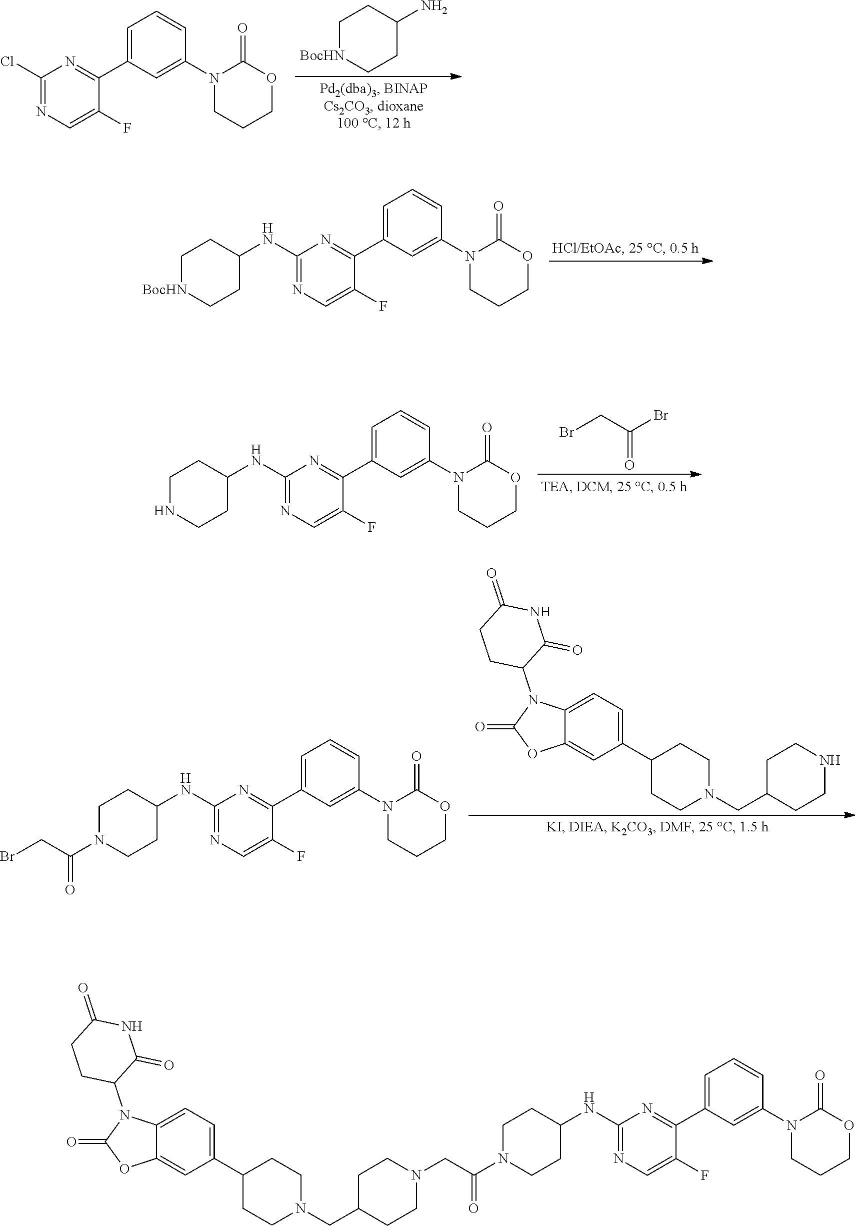

Synthesis of tert-butyl 4-((5-fluoro-4-(3-(2-oxo-1,3-oxazinan-3-yl)phenyl)pyrimidin-2-yl)amino)piperidine-1-carboxylate. A mixture of 3-[3-(2-chloro-5-fluoro-pyrimidin-4-yl)phenyl]-1,3-oxazinan-2-one (1.00 g, 3.25 mmol, 1.00 eq), tert-butyl 4-aminopiperidine-1-carboxylate (846 mg, 4.22 mmol, 1.30 eq), Cs₂CO₃ (3.18 g, 9.75 mmol, 3.00 eq), Pd₂(dba)₃ (297 mg, 324 μmol, 0.100 eq) and BINAP (202 mg, 324 μmol, 0.100 eq) in dioxane (15 mL) was degassed and purged with N₂ for 3 times at 25 °C, and then the mixture was stirred at 100 °C for 12 h under N₂ atmosphere. LC-MS showed the desired mass was detected. The mixture was diluted with water (20 mL), and extracted with EtOAc (20 mL*3). The combined organic layers were washed with brine (10 mL), dried over Na₂SO₄, filtered and concentrated under reduced pressure. The residue was purified by column chromatography (SiO₂, Petroleum ether:Ethyl acetate=1:0 to 0:1) to give the title compound (800 mg, 1.53 mmol, 46.9% yield, 90.0% purity) as a yellow solid. LCMS (ES⁺): m/z 472.3 [M+H]⁺.

Synthesis of 3-(3-(5-fluoro-2-(piperidin-4-ylamino)pyrimidin-4-yl)phenyl)-1,3-oxazinan-2-one. A solution of tert-butyl 4-[[5-fluoro-4-[3-(2-oxo-1,3-oxazinan-3-yl)phenyl]pyrimidin-2-yl]amino]piperidine-1-carboxylate (300 mg, 636 μmol, 1.00 eq) in HCV/EtOAc (4 M, 3 mL, 18.8 eq) was stirred at 25 °C for 0.5 h. LC-MS showed the desired mass was detected. The reaction mixture was concentrated under reduced pressure to give the title compound (360 mg, crude, HCl) as a yellow solid. LCMS (ES⁺): m/z 372.3 [M+H]⁺.

Synthesis of 3-(3-(2-((1-(2-bromoacetyl)piperidin-4-yl)amino)-5-fluoropyrimidin-4-yl)phenyl)-1,3-oxazinan-2-one. To a solution of 3-[3-[5-fluoro-2-(4-piperidylamino)pyrimidin-4-yl]phenyl]-1,3-oxazinan-2-one (300 mg, 735 μmol, 1.00 eq, HCl) in DCM (3 mL) was added TEA (223 mg, 2.21 mmol, 307 μL, 3.00 eq) until pH-8 at 25 °C, and 2-bromoacetyl bromide (148 mg, 735 μmol, 64.0 μL, 1.00 eq) was added at 25 °C under N₂ atmosphere. The mixture was stirred at 25 °C for 0.5 h under N₂ atmosphere. LC-MS showed the desired mass was detected. The residue was purified by prep-TLC (SiO₂, Dichloromethane:Tetrahydrofuran=0:1) to give the title compound (170 mg, crude) as a yellow solid. LCMS (ES⁺): m/z 492.3 [M+H]⁺.

Synthesis of 3-(6-(1-((1-(2-(4-((5-fluoro-4-(3-(2-oxo-1,3-oxazinan-3-yl)phenyl)pyrimidin-2-yl)amino)piperidin-1-yl)-2-oxoethyl)piperidin-4-yl)methyl)piperidin-4-yl)-2-oxobenzo[d]oxazol-3 (2H)-yl)piperidine-2,6-dione (Compound 371). To a solution of 3-(2-oxo-6-(1-(piperidin-4-ylmethyl)piperidin-4-yl)benzo[d]oxazol-3 (2H)-yl)piperidine-2,6-dione (121 mg, 284 μmol, 1.00 eq) in DMF (3 mL) was added DIEA (110 mg, 853 μmol, 148 L, 3.00 eq) until pH-8 at 25 °C. The mixture was stirred at 25 °C for 0.5 h. Then 3-[3-[2-[[1-(2-bromoacetyl)-4-piperidyl]amino]-5-fluoro-pyrimidin-4-yl]phenyl]-1,3-oxazinan-2-one (140 mg, 284 μmol, 1.00 eq), K₂CO₃ (117 mg, 853 μmol, 3.00 eq) and KI (47.2 mg, 284 μmol, 1.00 eq) was added into the solution. The mixture was stirred at 25 °C for 1 h. LC-MS showed the desired mass was detected. The reaction mixture was filtered and concentrated under reduced pressure. The residue was purified by prep-HPLC (column: 3_Phenomenex Luna C18 75*30 mm*3 um; mobile phase: [H₂O (0.2% FA)-ACN]; gradient: 1%-25% B over 8.0 min) to give the title compound (Compound 371) (22.5 mg, 26.5 μmol, 9.33% yield, 98.8% purity) as a white solid. LCMS (ES⁺): m/z 838.5 [M+H]⁺.

### Example 430. Synthesis of trans-2-(4-((4-(3-(2,6-dioxopiperidin-3-yl)-2-oxo-2,3-dihydrobenzo[d]oxazol-6-yl)piperidin-1-yl)methyl)piperidin-1-yl)-N-(4-((5-fluoro-4-(3-(2-oxo-1,3-oxazinan-3-yl)phenyl)pyrimidin-2-yl)amino)cyclohexyl)acetamide (Compound 362)

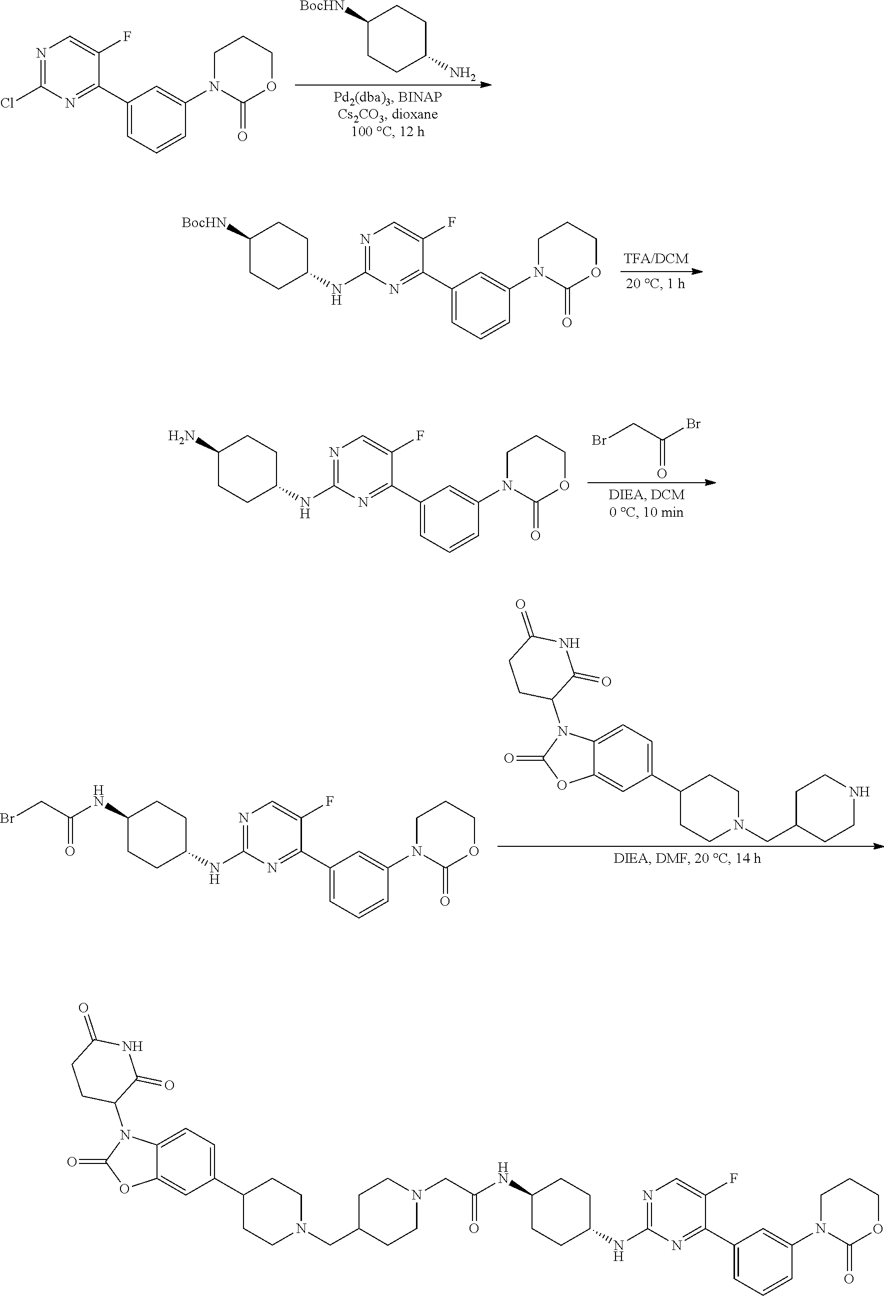

Synthesis of trans-tert-butyl (4-((5-fluoro-4-(3-(2-oxo-1,3-oxazinan-3-yl)phenyl)pyrimidin-2-yl)amino)cyclohexyl)carbamate. To a solution of 3-(3-(2-chloro-5-fluoropyrimidin-4-yl)phenyl)-1,3-oxazinan-2-one (500 mg, 1.62 mmol, 1.00 eq) and trans-tert-butyl (4-aminocyclohexyl)carbamate (522 mg, 2.44 mmol, 1.50 eq) in dioxane (20 mL) were added Cs₂CO₃ (1.06 g, 3.25 mmol, 2.00 eq), Pd₂(dba)₃ (119 mg, 130 μmol, 0.0800 eq) and BINAP (81.0 mg, 130 μmol, 0.0800 eq) at 20 °C under N₂ atmosphere. The mixture was stirred at 100 °C for 12 h under N₂ atmosphere. LCMS showed 3-(3-(2-chloro-5-fluoropyrimidin-4-yl)phenyl)-1,3-oxazinan-2-one was consumed and the desired mass was detected. To the mixture was added H₂O (20 mL) and the mixture was extracted with EtOAc (10 mL*3). The combined organic layers were washed with brine (10 mL*3), dried over Na₂SO₄, filtered and the filtrate was concentrated under reduced pressure to give a residue. The residue was purified by prep-TLC (SiO₂, PE/EtOAc=1/2) to give the title compound (400 mg, 824 μmol, 50.7% yield) as a yellow solid. LCMS (ES+): m/z 486.3 [M+H]⁺.

Synthesis of trans-3-(3-(2-((4-aminocyclohexyl)amino)-5-fluoropyrimidin-4-yl)phenyl)-1,3-oxazinan-2-one. A solution of trans-tert-butyl (4-((5-fluoro-4-(3-(2-oxo-1,3-oxazinan-3-yl)phenyl)pyrimidin-2-yl)amino)cyclohexyl)carbamate (400 mg, 824 μmol, 1.00 eq) in DCM (5 mL) and TFA (1 mL) was stirred at 20 °C for 1 h. LCMS showed trans-tert-butyl (4-((5-fluoro-4-(3-(2-oxo-1,3-oxazinan-3-yl)phenyl)pyrimidin-2-yl)amino)cyclohexyl)carbamate was consumed and the desired mass was detected. The mixture was concentrated under reduced pressure to give the title compound (400 mg, crude, TFA) as a yellow oil. LCMS (ES+): m/z 386.3 [M+H]⁺.

Synthesis of trans-2-bromo-N-(4-((5-fluoro-4-(3-(2-oxo-1,3-oxazinan-3-yl)phenyl)pyrimidin-2-yl)amino)cyclohexyl)acetamide. To a solution of trans-3-(3-(2-((4-aminocyclohexyl)amino)-5-fluoropyrimidin-4-yl)phenyl)-1,3-oxazinan-2-one (400 mg, 801 μmol, 1.00 eq, TFA) in DCM (5 mL) were added DIEA (414 mg, 3.20 mmol, 558 μL, 4.00 eq) and 2-bromoacetyl bromide (242 mg, 1.20 mmol, 105 μL, 1.50 eq) at 0 °C. The mixture was stirred at 0 °C for 10 min. LCMS showed trans-3-(3-(2-((4-aminocyclohexyl)amino)-5-fluoropyrimidin-4-yl)phenyl)-1,3-oxazinan-2-one was consumed and the desired mass was detected. The mixture was concentrated under reduced pressure to give a residue. The residue was purified by prep-TLC (SiO₂, PE/EtOAc=0/1) to give the title compound (350 mg, 691 μmol, 86.3% yield) as a yellow oil. LCMS (ES⁺): m/z 506.2 [M+H]⁺.

Synthesis of trans-2-(4-((4-(3-(2,6-dioxopiperidin-3-yl)-2-oxo-2,3-dihydrobenzo[d]oxazol-6-yl)piperidin-1-yl)methyl)piperidin-1-yl)-N-(4-((5-fluoro-4-(3-(2-oxo-1,3-oxazinan-3-yl)phenyl)pyrimidin-2-yl)amino)cyclohexyl)acetamide (Compound 362). To a solution of 3-(2-oxo-6-(1-(piperidin-4-ylmethyl)piperidin-4-yl)benzo[d]oxazol-3 (2H)-yl)piperidine-2,6-dione (384 mg, 711 μmol, 1.20 eq, TFA) and DIEA (384 mg, 2.97 mmol, 518 μL, 5.00 eq) in DMF (5 mL) was added trans-2-bromo-N-(4-((5-fluoro-4-(3-(2-oxo-1,3-oxazinan-3-yl)phenyl)pyrimidin-2-yl)amino)cyclohexyl)acetamide (300 mg, 592 μmol, 1.00 eq) at 20 °C, then the mixture was stirred at 20 °C for 14 h. LCMS showed trans-2-bromo-N-(4-((5-fluoro-4-(3-(2-oxo-1,3-oxazinan-3-yl)phenyl)pyrimidin-2-yl)amino)cyclohexyl)acetamide was consumed completely and the desired mass was detected. The mixture was concentrated under reduced pressure to give a residue. The residue was purified by prep-HPLC (column: Phenomenex Luna C18 75*30 mm*3 um; mobile phase: [H₂O (0.2% FA)-ACN]; gradient: 15%-55% B over 8.0 min) to give the title compound (Compound 362) (50.4 mg, 58.6 μmol, 9.89% yield, 99.1% purity) as a white solid. LCMS (ES⁺): m/z 852.4 [M+H]⁺

### Example 431. Synthesis of 3-(6-(1-(2-(1-((1R,3S)-3-((5-fluoro-4-(3-(2-oxopiperidin-1-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 356)

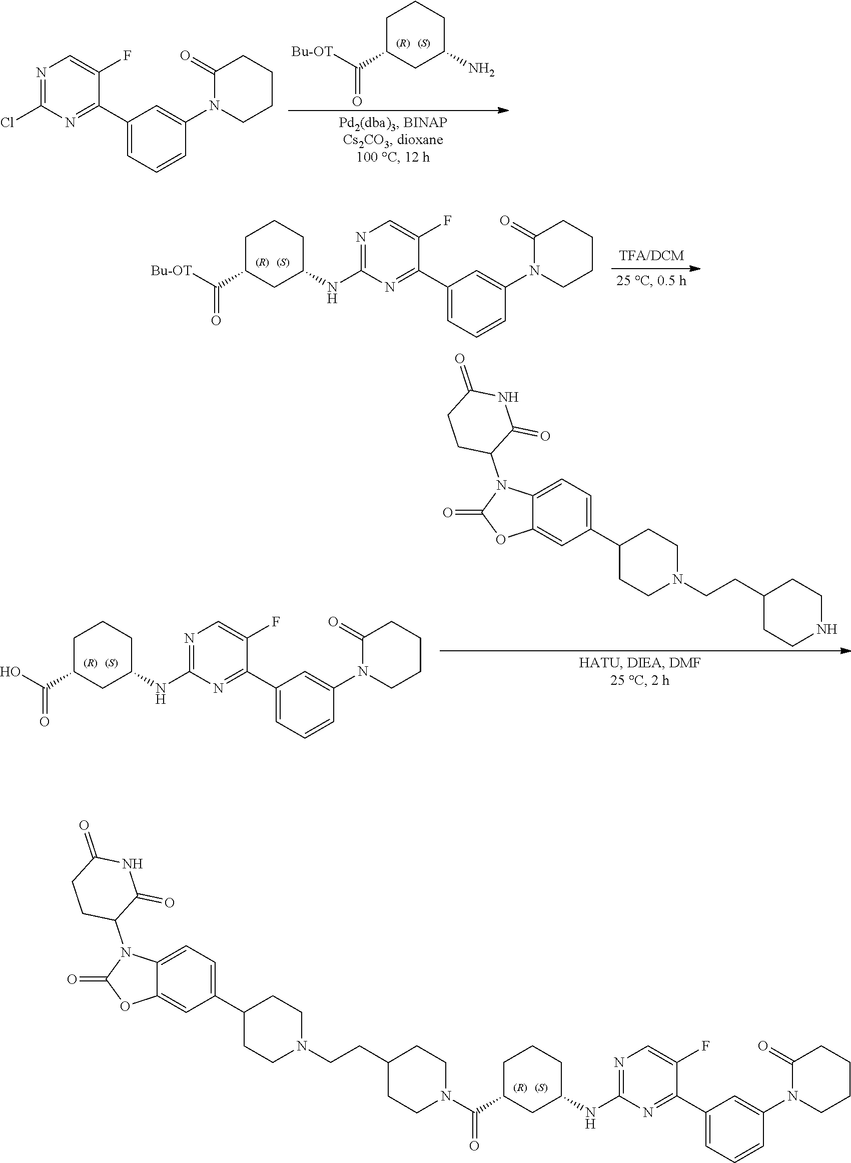

Synthesis of tert-butyl (1R,3S)-3-((5-fluoro-4-(3-(2-oxopiperidin-1-yl)phenyl)pyrimidin-2-yl)amino)cyclohexane-1-carboxylate. To a solution of 1-(3-(2-chloro-5-fluoropyrimidin-4-yl)phenyl)piperidin-2-one (500 mg, 1.64 mmol, 1.00 eq) and tert-butyl (1R,3S)-3-aminocyclohexane-1-carboxylate (326 mg, 1.64 mmol, 1.00 eq) in dioxane (15 mL) were added Pd₂(dba)₃ (150 mg, 164 μmol, 0.100 eq), BINAP (102 mg, 164 μmol, 0.100 eq) and Cs₂CO₃ (1.07 g, 3.27 mmol, 2.00 eq) at 25 °C under N₂. The mixture was stirred at 100 °C for 12 h under N₂. LCMS showed that 1-(3-(2-chloro-5-fluoropyrimidin-4-yl)phenyl)piperidin-2-one was consumed completely and the desired mass was detected. H₂O (15 mL) was added to the solution, then the mixture was extracted with EtOAc (10 mL*3), the combined organic layers were dried over Na₂SO₄, filtered and the filtrate was concentrated under reduced pressure to give a residue. The residue was purified by prep-TLC (SiO₂, PE/EtOAc=1/1) to give the title compound (470 mg, 1.00 mmol, 61.3% yield) as a brown oil. LCMS (ES⁺): m/z 469.4 [M+H]⁺.

Synthesis of (1R,3S)-3-((5-fluoro-4-(3-(2-oxopiperidin-1-yl)phenyl)pyrimidin-2-yl)amino)cyclohexane-1-carboxylic acid. To a solution of tert-butyl (1R,3S)-3-((5-fluoro-4-(3-(2-oxopiperidin-1-yl)phenyl)pyrimidin-2-yl)amino)cyclohexane-1-carboxylate (470 mg, 1.00 mmol, 1.00 eq) in DCM (9 mL) was added TFA (3 mL) at 25 °C. The mixture was stirred at 25 °C for 0.5 h. LCMS showed that tert-butyl (1R,3S)-3-((5-fluoro-4-(3-(2-oxopiperidin-1-yl)phenyl)pyrimidin-2-yl)amino)cyclohexane-1-carboxylate was consumed completely and the desired mass was detected. The mixture was concentrated under reduced pressure to give the title compound (413 mg, crude) as a yellow oil. LCMS (ES⁺): m/z 413.1 [M+H]⁺.

Synthesis of 3-(6-(1-(2-(1-((1R,3S)-3-((5-fluoro-4-(3-(2-oxopiperidin-1-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 356). To a solution of (1R,3S)-3-((5-fluoro-4-(3-(2-oxopiperidin-1-yl)phenyl)pyrimidin-2-yl)amino)cyclohexane-1-carboxylic acid (149 mg, 361 μmol, 1.00 eq) in DMF (4 mL) were added HATU (165 mg, 433 μmol, 1.20 eq) and DIEA (140 mg, 1.08 mmol, 188 μL, 3.00 eq) at 25 °C. Then 3-(2-oxo-6-(1-(2-(piperidin-4-yl)ethyl)piperidin-4-yl)benzo[d]oxazol-3 (2H)-yl)piperidine-2,6-dione (200 mg, 361 μmol, 1.00 eq, TFA) was added at 25 °C. The resulting mixture was stirred at 25 °C for 2 h. LCMS showed that (1R,3S)-3-((5-fluoro-4-(3-(2-oxopiperidin-1-yl)phenyl)pyrimidin-2-yl)amino)cyclohexane-1-carboxylic acid was consumed completely and the desired mass was detected. The mixture was concentrated under reduced pressure. The residue was purified by prep-HPLC (column: Phenomenex Luna C18 75*30 mm*3 um; mobile phase: [1H₂O (0.2% FA)-ACN]; gradient: 10%-50% B over 8.0 min) to give the title compound (Compound 356) (32.0 mg, 37.0 μmol, 10.3% yield, 96.4% purity) as a white solid. LCMS (ESI+): m/s 835.4 [M+H]⁺

### Example 432. Synthesis of 3-(6-(1-(2-(1-((1R,3S)-3-((5-fluoro-4-(3-(2-oxo-5-(trifluoromethyl)pyridin-1(2H)-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 357)

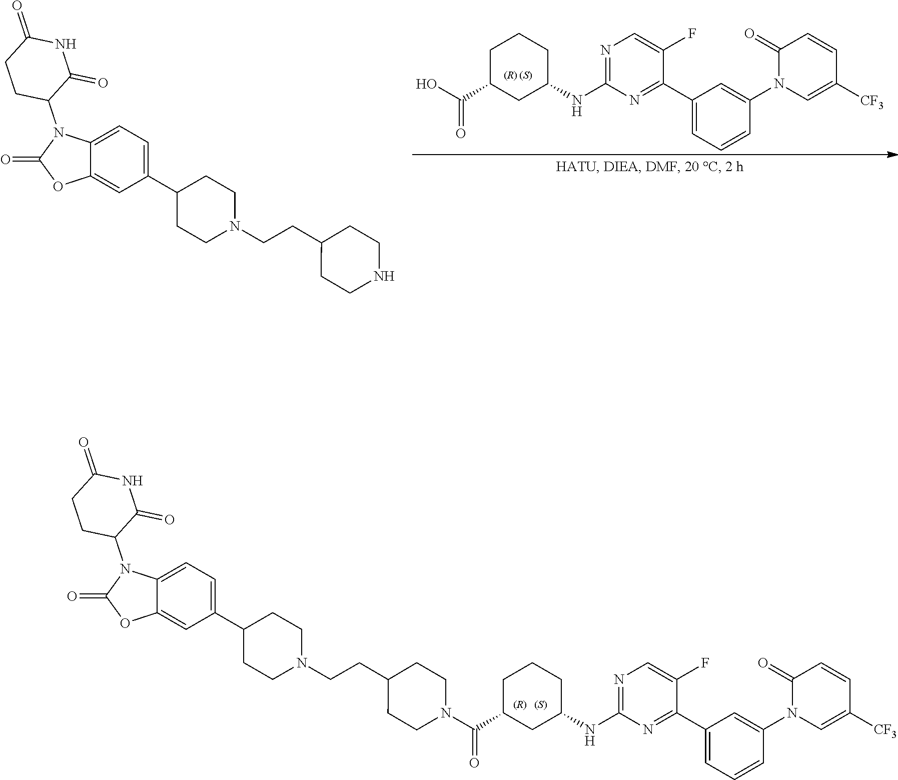

Synthesis of 3-(6-(1-(2-(1-((1R,3S)-3-((5-fluoro-4-(3-(2-oxo-5-(trifluoromethyl)pyridin-1(2H)-yl)phenyl)pyrimidin-2-yl)amino)cyclohexane-1-carbonyl)piperidin-4-yl)ethyl)piperidin-4-yl)-2-oxobenzo[d]oxazol-3 (2H)-yl)piperidine-2,6-dione (Compound 357). To a solution of (1R,3S)-3-((5-fluoro-4-(3-(2-oxo-5-(trifluoromethyl)pyridin-1(2H)-yl)phenyl)pyrimidin-2-yl)amino)cyclohexane-1-carboxylic acid (280 mg, 588 μmol, 1.00 eq) in DMF (5 mL) were added HATU (268 mg, 705 μmol, 1.20 eq) and DIEA (380 mg, 2.94 mmol, 512 μL, 5.00 eq) at 20 °C. Then to the mixture was added 3-(2-oxo-6-(1-(2-(piperidin-4-yl)ethyl)piperidin-4-yl)benzo[d]oxazol-3 (2H)-yl)piperidine-2,6-dione (326 mg, 588 μmol, 1.00 eq, TFA) at 20 °C. The mixture was stirred at 20 °C for 2 h. LCMS showed 3-(2-oxo-6-(1-(2-(piperidin-4-yl)ethyl)piperidin-4-yl)benzo[d]oxazol-3 (2H)-yl)piperidine-2,6-dione was consumed and the desired mass was detected. To the mixture was added H₂O (5 mL) and the mixture was extracted with EtOAc (5 mL*3). The combined organic layers were washed with brine (5 mL), dried over Na₂SO₄, filtered and the filtrate was concentrated under reduced pressure to give a residue. The residue was purified by prep-HPLC (column: Phenomenex Luna C18 75*30 mm*3 um; mobile phase: [H₂O (0.2% FA)-ACN]; gradient: 15%-55% B over 8.0 min) to give the title compound (Compound 357) (25.0 mg, 27.8 μmol, 4.73% yield, 100% purity, FA) as a white solid. LCMS (ES⁺): m/z 899.3 [M+H]⁺

### Example 433. Synthesis of 3-(6-(1-((1-(2-(4-((5-fluoro-4-(3-(2-oxooxazolidin-3-yl)phenyl)pyrimidin-2-yl)amino)piperidin-1-yl)-2-oxoethyl)piperidin-4-yl)methyl)piperidin-4-yl)-2-oxobenzo[d]oxazol-3 (2H)-yl)piperidine-2,6-dione (Compound 372)

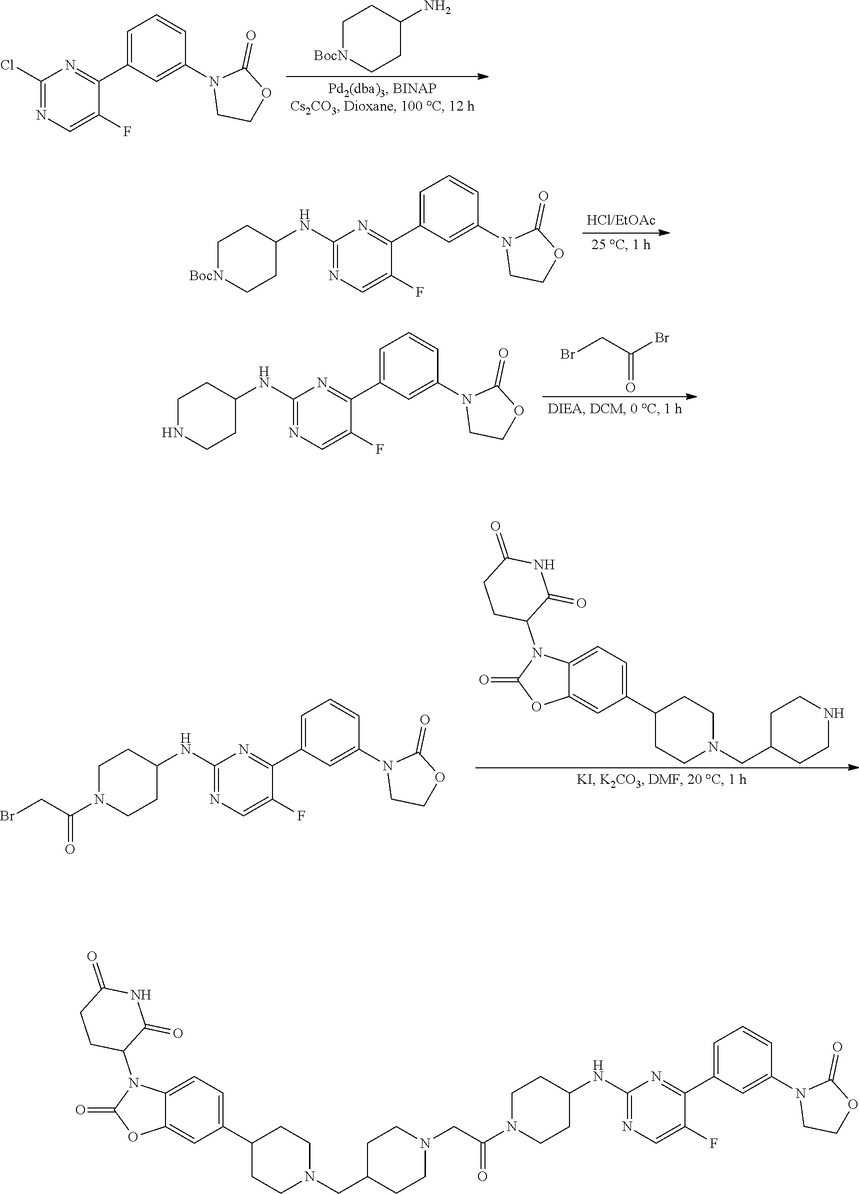

Synthesis of tert-butyl 4-((5-fluoro-4-(3-(2-oxooxazolidin-3-yl)phenyl)pyrimidin-2-yl)amino)piperidine-1-carboxylate. A mixture of 3-[3-(2-chloro-5-fluoro-pyrimidin-4-yl)phenyl]oxazolidin-2-one (600 mg, 2.04 mmol, 1.00 eq), tert-butyl 4-aminopiperidine-1-carboxylate (409 mg, 2.04 mmol, 1.00 eq), BINAP (127 mg, 204 μmol, 0.100 eq), Pd₂(dba)₃ (187 mg, 204 μmol, 0.10 eq) and Cs₂CO₃ (2.00 g, 6.13 mmol, 3.00 eq) in dioxane (10 mL) was degassed and purged with N₂ for 3 times at 20 °C, and then the mixture was stirred at 100 °C for 12 h under N₂ atmosphere. LC-MS showed the desired mass was detected. The reaction mixture was quenched by addition H₂O (20 mL), and then extracted with EtOAc (20 mL*3). The combined organic layers were dried over Na₂SO₄, filtered and concentrated under reduced pressure. The residue was purified by column chromatography (SiO₂, Petroleum ether:Ethyl acetate=20:1 to 3:1) to give the title compound (350 mg, 765 μmol, 37.5% yield) as a yellow solid. LCMS (ES⁺): m/z 458.4[M+H]⁺.

Synthesis of 3-[3-[5-fluoro-2-(4-piperidylamino) pyrimidin-4-yl]phenyl]oxazolidin-2-one. To a solution of tert-butyl 4-[[5-fluoro-4-[3-(2-oxooxazolidin-3-yl)phenyl]pyrimidin-2-yl]amino]piperidine-1-carboxylate (250 mg, 546 μmol, 1.00 eq) in HCl/EtOAc (4.00 M, 5.00 mL, 36.6 eq). The mixture was stirred at 20 °C for 1 h. LC-MS showed the desired mass was detected. The reaction mixture was concentrated under reduced pressure to give the title compound (140 mg, crude, HCl) as a yellow oil. LCMS (ES⁺): m/z 358.4[M+H]⁺.

Synthesis of 3-[3-[2-[[1-(2-bromoacetyl)-4-piperidyl]amino]-5-fluoro-pyrimidin-4-yl]phenyl]oxazolidin-2-one. To a solution of 2-bromoacetyl bromide (220 mg, 1.09 mmol, 95.1 μL, 3.00 eq) in DCM (1 mL) was added DIEA (94.0 mg, 727 μmol, 126 μL, 2.00 eq) and 3-[3-[5-fluoro-2-(4-piperidylamino)pyrimidin-4-yl]phenyl]oxazolidin-2-one (130 mg, 363 μmol, 1.00 eq, HCl) at 0 °C. The mixture was stirred at 0 °C for 1 h. LC-MS showed the desired mass was detected. The reaction mixture was concentrated under reduced pressure. The residue was purified by prep-TLC (SiO₂, Petroleum ether:Ethyl acetate=3:1, Rf=0.28) to give the title compound (135 mg, 282 μmol, 77.6% yield) as a yellow oil. LCMS (ES⁺): m/z 478.1 [M+H]⁺

Synthesis of 3-(6-(1-((1-(2-(4-((5-fluoro-4-(3-(2-oxooxazolidin-3-yl)phenyl)pyrimidin-2-yl)amino)piperidin-1-yl)-2-oxoethyl)piperidin-4-yl)methyl)piperidin-4-yl)-2-oxobenzo[d]oxazol-3 (2H)-yl)piperidine-2,6-dione (Compound 372). To a solution of 3-[3-[2-[[1-(2-bromoacetyl)-4-piperidyl]amino]-5-fluoro-pyrimidin-4-yl]phenyl]oxazolidin-2-one (130 mg, 271 μmol, 1.00 eq) in DMF (1 mL) was added 3-(2-oxo-6-(1-(piperidin-4-ylmethyl)piperidin-4-yl)benzo[d]oxazol-3 (2H)-yl)piperidine-2,6-dione (115 mg, 214 μmol, 0.789 eq, TFA), KI (45.1 mg, 271 μmol, 1.00 eq) and K₂CO₃ (112 mg, 815 μmol, 3.00 eq) at 20 °C. The mixture was stirred at 20 °C for 1 h. LC-MS showed the desired mass was detected. The reaction mixture was quenched by addition H₂O 10 mL, and then extracted with EtOAc (10 mL*3). The combined organic layers were dried over Na₂SO₄, filtered and concentrated under reduced pressure. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to give the title compound (Compound 372) (49.5 mg, 56.9 μmol, 20.9% yield, 94.8% purity) as a yellow solid. LCMS (ES⁺): m/z 824.5 [M+H]⁺

### Example 434. Synthesis of trans-2-[4-[[4-[3-(2,6-dioxo-3-piperidyl)-2-oxo-1,3-benzoxazol-6-yl]-1-piperidyl]methyl]-1-piperidyl]-N-[4-[[5-fluoro-4-[3-(2-oxooxazolidin-3-yl)phenyl]pyrimidin-2-yl]amino]cyclohexyl]acetamide (Compound 363)

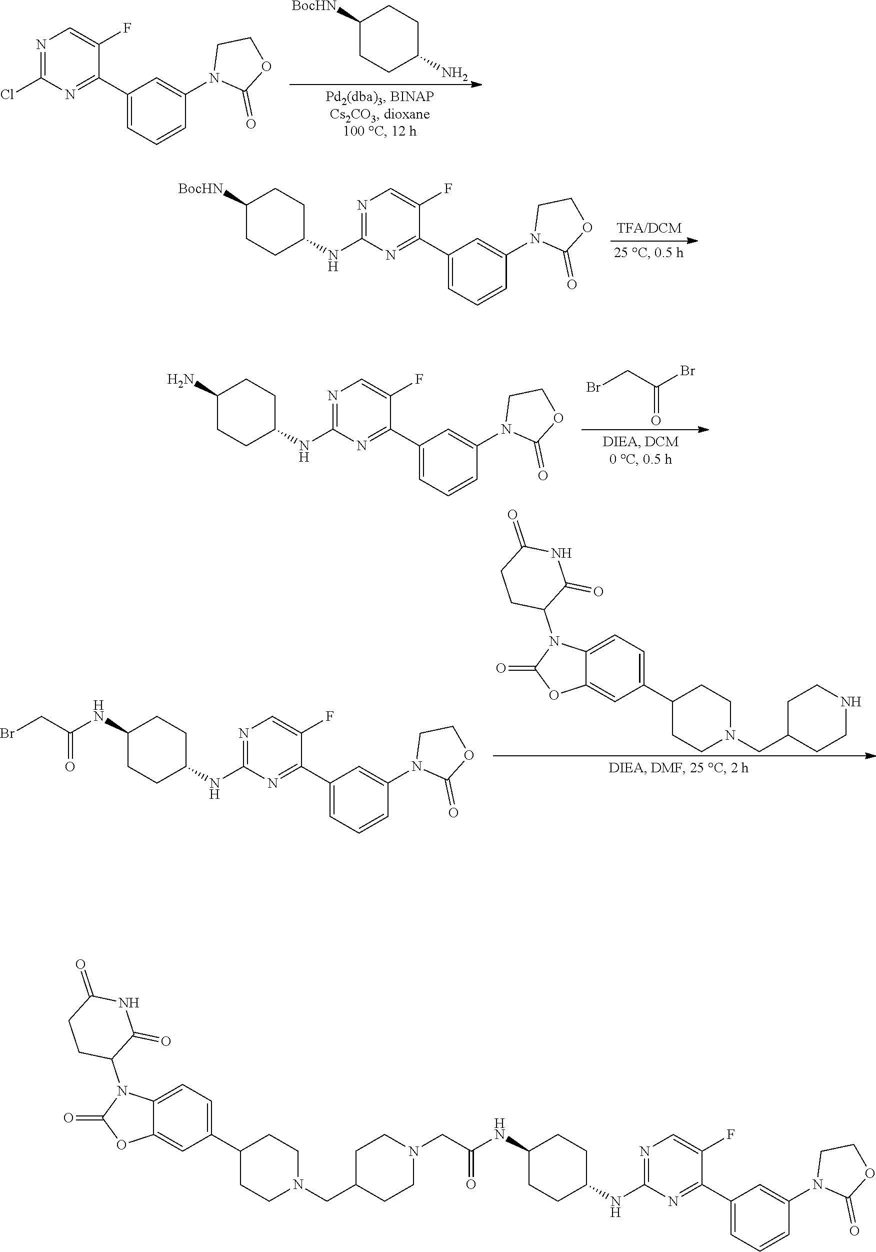

Synthesis of trans-tert-butyl-(4-((5-fluoro-4-(3-(2-oxooxazolidin-3-yl)phenyl)pyrimidin-2-yl)amino)cyclohexyl)carbamate. To a solution of trans-tert-butyl (4-aminocyclohexyl)carbamate (1.09 g, 5.11 mmol, 1.00 eq) and 3-(3-(2-chloro-5-fluoropyrimidin-4-yl)phenyl)oxazolidin-2-one (1.50 g, 5.11 mmol, 1.00 eq) in dioxane (30 mL) were added Pd₂(dba)₃ (468 mg, 511 μmol, 0.100 eq), BINAP (318 mg, 511 μmol, 0.100 eq) and Cs₂CO₃ (3.33 g, 10.2 mmol, 2.00 eq) at 25 °C under N₂. The mixture was stirred at 100 °C for 12 h under N₂. LCMS showed that 3-(3-(2-chloro-5-fluoropyrimidin-4-yl)phenyl)oxazolidin-2-one was consumed completely and the desired mass was detected. H₂O (30 mL) was added to the solution, then the mixture was extracted with EtOAc (20 mL*3), the combined organic layers were dried over Na₂SO₄, filtered and the filtrate was concentrated under reduced pressure to give a residue. The residue was purified by MPLC (SiO₂, Petroleum ether/Ethyl acetate=1/0 to 1/1) to give the title compound (900 mg, 1.91 mmol, 37.4% yield) as a yellow solid. LCMS (ES⁺): m/z 472.4 [M+H]⁺.

Synthesis of trans-3-(3-(2-((4-aminocyclohexyl)amino)-5-fluoropyrimidin-4-yl)phenyl)oxazolidin-2-one. To a solution of trans-tert-butyl (4-((5-fluoro-4-(3-(2-oxooxazolidin-3-yl)phenyl)pyrimidin-2-yl)amino)cyclohexyl)carbamate (300 mg, 636 μmol, 1.00 eq) in DCM (6 mL) was added TFA (2 mL) at 25 °C. The resulting mixture was stirred at 25 °C for 0.5 h. LCMS showed that trans-tert-butyl (4-((5-fluoro-4-(3-(2-oxooxazolidin-3-yl)phenyl)pyrimidin-2-yl)amino)cyclohexyl)carbamate was consumed completely and the desired mass was detected. The mixture was concentrated under reduced pressure to give the title compound (900 mg, crude, TFA) as a yellow oil. LCMS (ES⁺): m/z 372.2 [M+H]⁺.

Synthesis of trans-2-bromo-N-(4-((5-fluoro-4-(3-(2-oxooxazolidin-3-yl)phenyl)pyrimidin-2-yl)amino)cyclohexyl)acetamide. To a solution of trans-3-(3-(2-((4-aminocyclohexyl)amino)-5-fluoropyrimidin-4-yl)phenyl)oxazolidin-2-one (300 mg, 618 μmol, 1.00 eq, TFA) in DCM (6 mL) was added DIEA (240 mg, 1.86 mmol, 323 μL, 3.00 eq) at 25 °C, then 2-bromoacetyl bromide (150 mg, 742 μmol, 64.6 μL, 1.20 eq) was added to the mixture at 0 °C. The resulting mixture was stirred at 0 °C for 0.5 h. LCMS showed that trans-3-(3-(2-((4-aminocyclohexyl)amino)-5-fluoropyrimidin-4-yl)phenyl)oxazolidin-2-one was consumed completely and the desired mass was detected. The mixture was concentrated under reduced pressure to give the title compound (360 mg, crude) as a yellow oil. LCMS (ES⁺): m/z 492.2 [M+H]⁺.

Synthesis of trans-2-[4-[[4-[3-(2,6-dioxo-3-piperidyl)-2-oxo-1,3-benzoxazol-6-yl]-1-piperidyl]methyl]-1-piperidyl]-N-[4-[[5-fluoro-4-[3-(2-oxooxazolidin-3-yl)phenyl]pyrimidin-2-yl]amino]cyclohexyl]acetamide (Compound 363). To a solution of trans-2-bromo-N-(4-((5-fluoro-4-(3-(2-oxooxazolidin-3-yl)phenyl)pyrimidin-2-yl)amino)cyclohexyl)acetamide (300 mg, 609 μmol, 1.00 eq) and 3-(2-oxo-6-(1-(piperidin-4-ylmethyl)piperidin-4-yl)benzo[d]oxazol-3 (2H)-yl)piperidine-2,6-dione (329 mg, 609 μmol, 1.00 eq, TFA) in DMF (5 mL) was added dropwise DIEA (236 mg, 1.83 mmol, 318 μL, 3.00 eq) at 0 °C. The mixture was stirred at 25 °C for 2 h. LCMS showed trans-2-bromo-N-(4-((5-fluoro-4-(3-(2-oxooxazolidin-3-yl)phenyl)pyrimidin-2-yl)amino)cyclohexyl)acetamide was consumed completely and the desired mass was detected. The residue was purified by prep-HPLC (column: Waters Xbridge Prep OBD C18 150*40 mm*10 um; mobile phase: [H₂O (10 mM NH₄HCO₃)-ACN]; gradient: 30%-65% B over 8.0 min) to give the title compound (Compound 363) (39.8 mg, 45.1 μmol, 7.40% yield, 95.0% purity) as a yellow solid. LCMS (ES⁺): m/z 838.4 [M+H]⁺

### Example 435. Synthesis of trans-3-(6-(4-((1-(4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperazin-1-yl)-2-oxobenzo[d]oxazol-3 (2H)-yl)piperidine-2,6-dione (Compound 398)

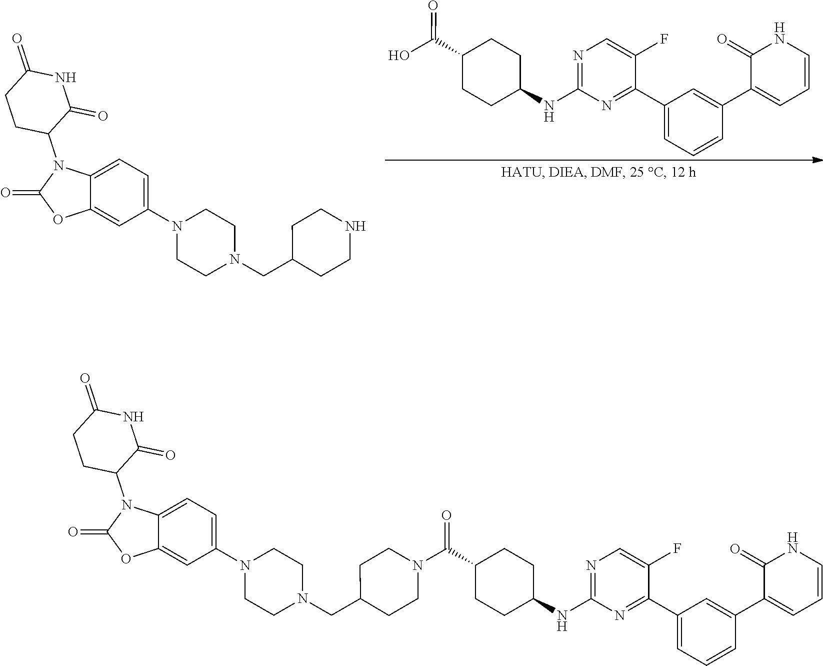 [SMILES: O=C1CCC(n2c(=O)oc3cc(N4CCN(CC5CCNCC5)CC4)ccc32)C(=O)N1 ; O=c1[nH]cccc1-c1cccc(-c2nc(N[C@H]3CC[C@H](C(=O)O)CC3)ncc2F)c1 ; O=C1CCC(n2c(=O)oc3cc(N4CCN(CC5CCN(C(=O)[C@H]6CC[C@H](Nc7ncc(F)c(-c8cccc(-c9ccc[nH]c9=O)c8)n7)CC6)CC5)CC4)ccc32)C(=O)N1]

Synthesis of trans-3-(6-(4-((1-(4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperazin-1-yl)-2-oxobenzo[d]oxazol-3 (2H)-yl)piperidine-2,6-dione (Compound 398). To a solution of 3-(2-oxo-6-(4-(piperidin-4-ylmethyl)piperazin-1-yl)benzo[d]oxazol-3 (2H)-yl)piperidine-2,6-dione (300 mg, 554 μmol, 1.00 eq, TFA) in DMF (2 mL) was added DIEA (71.6 mg, 554 mol, 96.4 μL, 1.00 eq) until pH~7. To a solution of trans-4-[[5-fluoro-4-[3-(2-oxo-1H-pyridin-3-yl)phenyl]pyrimidin-2-yl]amino]cyclohexanecarboxylic acid (113 mg, 277 μmol, 0.500 eq) in DMF (2 mL) was added DIEA (71.6 mg, 554 μmol, 96.4 μL, 1.00 eq) until pH~7. Then HATU (210 mg, 554 μmol, 1.00 eq) was added to the mixture of the two solutions. The mixture was stirred at 25 °C for 12 h. LC-MS showed the desired mass was detected. The residue was purified by prep-HPLC (column: Phenomenex luna C18 100*40 mm*5 um; mobile phase: [H₂O (0.2% FA)-ACN]; gradient: 15%-30% B over 8.0 min) to give the title compound (Compound 398) (62.9 mg, 76.5 μmol, 13.8% yield, 99.5% purity) as a white solid. LCMS (ES⁺): m/z 818.3 [M+H]⁺.

### Example 436. Synthesis of 3-(6-(4-((1-(trans-4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperazin-1-yl)-1-methyl-1H-indazol-3-yl)piperidine-2,6-dione (Compound 417)

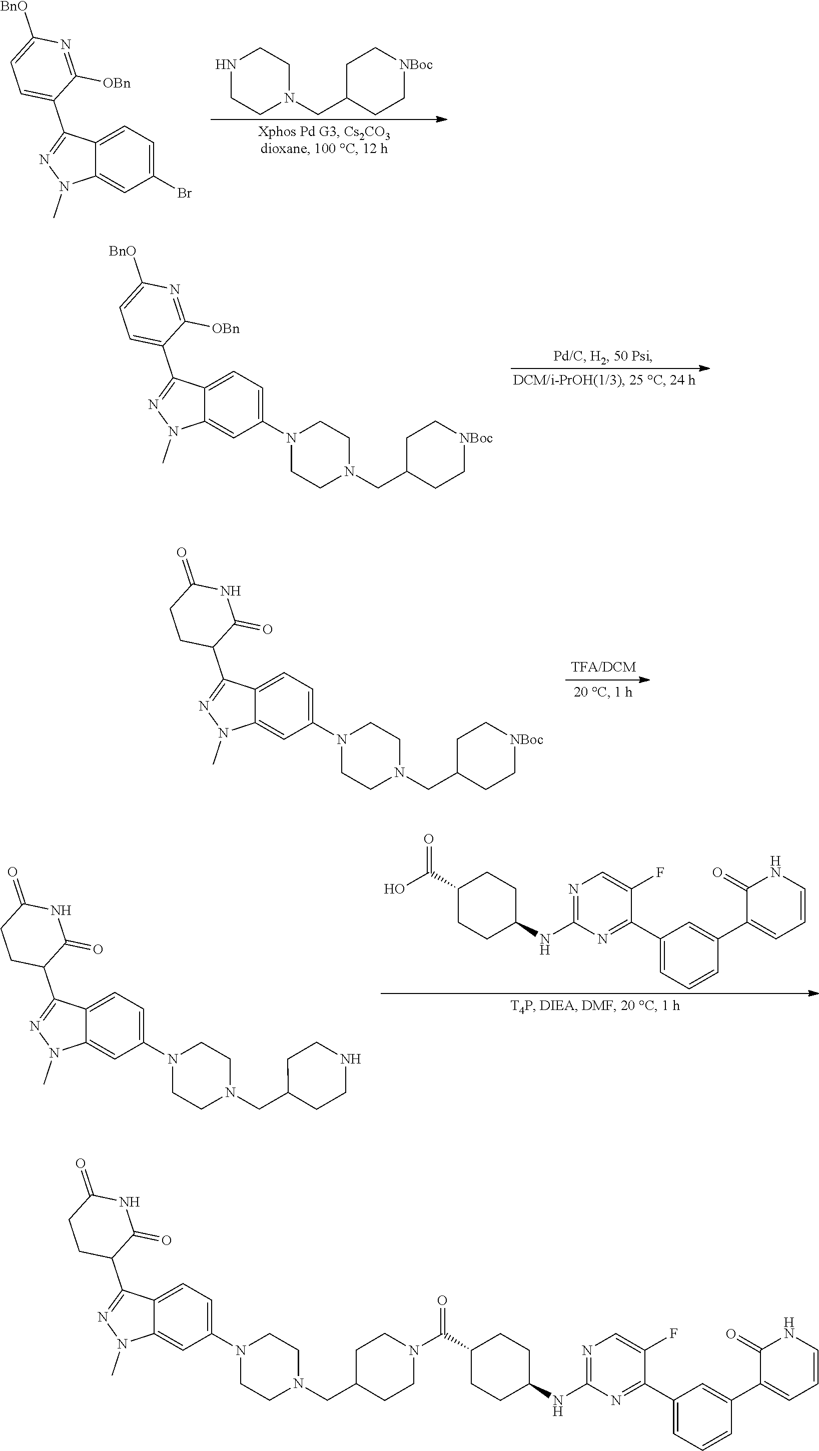

Synthesis of tert-butyl 4-((4-(3-(2,6-bis(benzyloxy)pyridin-3-yl)-1-methyl-1H-indazol-6-yl)piperazin-1-yl)methyl)piperidine-1-carboxylate. The reaction was set up in three batches. A mixture of 3-(2,6-bis(benzyloxy)pyridin-3-yl)-6-bromo-1-methyl-1H-indazole (5.00 g, 9.99 mmol, 1.00 eq), tert-butyl 4-(piperazin-1-ylmethyl)piperidine-1-carboxylate (3.40 g, 11.9 mmol, 1.20 eq), Cs₂CO₃ (6.51 g, 19.9 mmol, 2.00 eq), XPhos Pd G₃ (845 mg, 999 μmol, 0.100 eq) in dioxane (100 mL) was degassed at 20 °C and purged with N₂ for 3 times, and then the mixture was stirred at 100 °C for 12 h under N₂ atmosphere. LC-MS showed the desired mass was detected. Two batches in parallel were combined and the reaction mixture was quenched H₂O 100 mL and extracted with EtOAc 100 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (13.0 g, 12.0 mmol, 60.1% yield, 65.0% purity) as a black solid. LCMS (ES⁺): m/z 703.5 [M+H]⁺.

Synthesis of tert-butyl 4-((4-(3-(2,6-dioxopiperidin-3-yl)-1-methyl-1H-indazol-6-yl)piperazin-1-yl)methyl)piperidine-1-carboxylate. To a solution of tert-butyl 4-[[4-[3-(2,6-dibenzyloxy-3-pyridyl)-1-methyl-indazol-6-yl]piperazin-1-yl]methyl]piperidine-1-carboxylate (10.0 g, 14.2 mmol, 1.00 eq) in DCM (30 mL) and propan-2-ol (100 mL) was added 10% Pd/C (3.03 g, 14.2 mmol, 50% purity, 1.00 eq) under N₂ atmosphere at 25 °C. The suspension was degassed and purged with H₂ for 3 times. The mixture was stirred under H₂ (50 Psi) at 25 °C for 24 h. LC-MS showed the desired mass was detected. The reaction mixture was filtered and concentrated under reduced pressure. The residue was purified by column chromatography (SiO₂, Petroleum ether:Ethyl acetate=1:0 to 0:1) to give the title compound (4.00 g, 5.21 mmol, 36.6% yield, 68.3% purity) as a white solid. LCMS (ES⁺): m/z 525.3 [M+H]⁺.

Synthesis of 3-(1-methyl-6-(4-(piperidin-4-ylmethyl)piperazin-1-yl)-1H-indazol-3-yl)piperidine-2,6-dione. To a solution of tert-butyl 4-((4-(3-(2,6-dioxopiperidin-3-yl)-1-methyl-1H-indazol-6-yl)piperazin-1-yl)methyl)piperidine-1-carboxylate (1.00 g, 1.91 mmol, 1.00 eq) in DCM (10 mL) was added TFA (6.14 g, 53.8 mmol, 4.00 mL, 28.2 eq) at 20 °C. The mixture was stirred at 20 °C for 1 h. LC-MS showed the desired mass was detected. The reaction mixture was concentrated under reduced pressure to give the title compound (1.50 g, crude, TFA) as a black oil. LCMS (ES⁺): m/z 425.3 [M+H]⁺.

Synthesis of 3-(6-(4-((1-(trans-4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperazin-1-yl)-1-methyl-1H-indazol-3-yl)piperidine-2,6-dione (Compound 417). To a solution of 3-(1-methyl-6-(4-(piperidin-4-ylmethyl)piperazin-1-yl)-1H-indazol-3-yl)piperidine-2,6-dione (400 mg, 742 μmol, 1.00 eq, TFA) and trans-4-((5-fluoro-4-(3-(2-oxo-1,2-dihydropyridin-3-yl)phenyl)pyrimidin-2-yl)amino)cyclohexane-1-carboxylic acid (303 mg, 742 μmol, 1.00 eq) in DMF (5 mL) was added DIEA (191 mg, 1.49 mmol, 258 μL, 2.00 eq) and T₄P (267 mg, 742 μmol, 1.00 eq) at 20 °C. The mixture was stirred at 20 °C for 1 h. LC-MS showed the desired mass was detected. The reaction mixture was concentrated under reduced pressure. The residue was purified by prep-HPLC (column: Phenomenex Gemini-NX 80*40 mm*3 um; mobile phase: [H₂O (10 mM NH₄HCO₃)-ACN]; gradient: 25%-55% B over 8.0 min) to give the title compound (Compound 417) (37.0 mg, 45.4 μmol, 6.11% yield, 100% purity) as a light yellow solid. LCMS (ES⁺): m/z 815.3 [M+H]⁺.

### Example 437. Synthesis of trans-3-((3-fluoro-4-(4-((1-(4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperidin-1-yl)phenyl)amino)piperidine-2,6-dione (Compound 407)

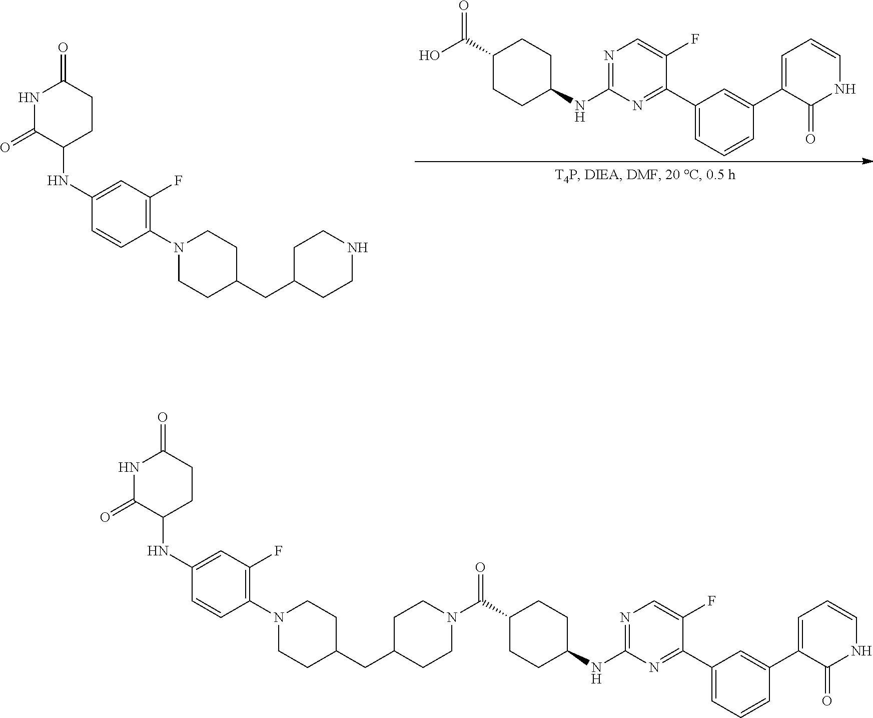 [SMILES: O=C1CCC(Nc2ccc(N3CCC(CC4CCNCC4)CC3)c(F)c2)C(=O)N1 ; O=c1[nH]cccc1-c1cccc(-c2nc(N[C@H]3CC[C@H](C(=O)O)CC3)ncc2F)c1 ; O=C1CCC(Nc2ccc(N3CCC(CC4CCN(C(=O)[C@H]5CC[C@H](Nc6ncc(F)c(-c7cccc(-c8ccc[nH]c8=O)c7)n6)CC5)CC4)CC3)c(F)c2)C(=O)N1]

Synthesis of trans-3-((3-fluoro-4-(4-((1-(4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperidin-1-yl)phenyl)amino)piperidine-2,6-dione (Compound 407). To a solution of trans-4-((5-fluoro-4-(3-(2-oxo-1,2-dihydropyridin-3-yl)phenyl)pyrimidin-2-yl)amino)cyclohexane-1-carboxylic acid (159 mg, 389 μmol, 0.300 eq) in DMF (0.4 mL) were added DIEA (335 mg, 2.59 mmol, 452 μL, 2.00 eq) and T₄P (514 mg, 1.43 mmol, 1.10 eq) at 20 °C, then 3-((3-fluoro-4-(4-(piperidin-4-ylmethyl)piperidin-1-yl)phenyl)amino)piperidine-2,6-dione (670 mg, 1.30 mmol, 1.00 eq, TFA) was added to the mixture, the mixture was stirred at 20 °C for 0.5 h. LCMS showed 3-((3-fluoro-4-(4-(piperidin-4-ylmethyl)piperidin-1-yl)phenyl)amino)piperidine-2,6-dione was consumed and the desired target MS was detected. The mixture was concentrated under reduced pressure to give a residue. The residue was purified by prep-HPLC (column: Waters Xbridge Prep OBD C18 150*40 mm*10 um; mobile phase: [1H₂O (10 mM NH₄HCO₃)-ACN]; gradient: 35%-65% B over 8.0 min) to give title compound (Compound 407) (34.6 mg, 42.3 μmol, 3.26% yield, 97.0% purity) as a white solid. LCMS (ES⁺): m/z 793.4 [M+H]⁺

### Example 438. Synthesis of trans-3-((3-fluoro-4-(1-((1-(4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperidin-4-yl)phenyl)amino)piperidine-2,6-dione (Compound 384)

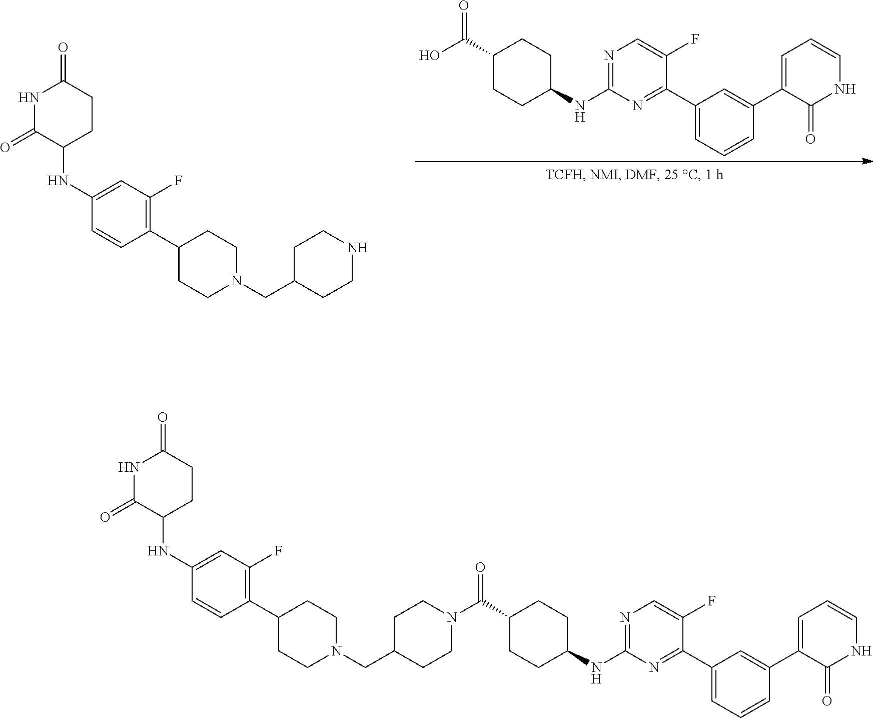 [SMILES: O=C1CCC(Nc2ccc(C3CCN(CC4CCNCC4)CC3)c(F)c2)C(=O)N1 ; O=c1[nH]cccc1-c1cccc(-c2nc(N[C@H]3CC[C@H](C(=O)O)CC3)ncc2F)c1 ; O=C1CCC(Nc2ccc(C3CCN(CC4CCN(C(=O)[C@H]5CC[C@H](Nc6ncc(F)c(-c7cccc(-c8ccc[nH]c8=O)c7)n6)CC5)CC4)CC3)c(F)c2)C(=O)N1]

Synthesis of trans-3-((3-fluoro-4-(1-((1-(4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperidin-4-yl)phenyl)amino)piperidine-2,6-dione (Compound 384). To a solution of trans-4-((5-fluoro-4-(3-(2-oxo-1,2-dihydropyridin-3-yl)phenyl)pyrimidin-2-yl)amino)cyclohexane-1-carboxylic acid (1.08 g, 2.64 mmol, 1.00 eq) in DMF (20 mL) were added TCFH (890 mg, 3.17 mmol, 1.20 eq) and NMI (1.30 g, 13.2 mmol, 1.06 mL, 5.00 eq) at 25 °C, then to the mixture was added 3-((3-fluoro-4-(1-(piperidin-4-ylmethyl)piperidin-4-yl)phenyl)amino)piperidine-2,6-dione (1.50 g, 2.91 mmol, 1.10 eq, TFA) at 25 °C, the mixture was stirred at 25 °C for 1 h. LCMS showed 3-((3-fluoro-4-(1-(piperidin-4-ylmethyl)piperidin-4-yl)phenyl)amino)piperidine-2,6-dione was consumed completely and the desired mass was detected. The mixture was diluted with H₂O (20 mL) and then extracted with EtOAc (20 mL*3). The combined organic layers were dried over Na₂SO₄, filtered and the filtrate was concentrated under reduced pressure to give a residue. The residue was purified by neutral prep-HPLC (column: Waters Xbridge Prep OBD C18 150*40 mm*10 um; mobile phase: [H₂O (10 mM NH₄HCO₃)-ACN]; gradient: 35%-60% B over 8.0 min) to give the title compound (Compound 384) (102 mg, 121 μmol, 4.59% yield, 93.9% purity) as a white solid. LCMS (ES⁺): m/z 793.4 [M+H]⁺.

### Example 439. Synthesis of trans-(R)-3-((3-fluoro-4-(1-((1-(-4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperidin-4-yl)phenyl)amino)piperidine-2,6-dione (Compound 421) and trans-(S)-3-((3-fluoro-4-(1-((1-(-4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperidin-4-yl)phenyl)amino)piperidine-2,6-dione (Compound 437) (The Two Isomers were Assigned Arbitrarily)

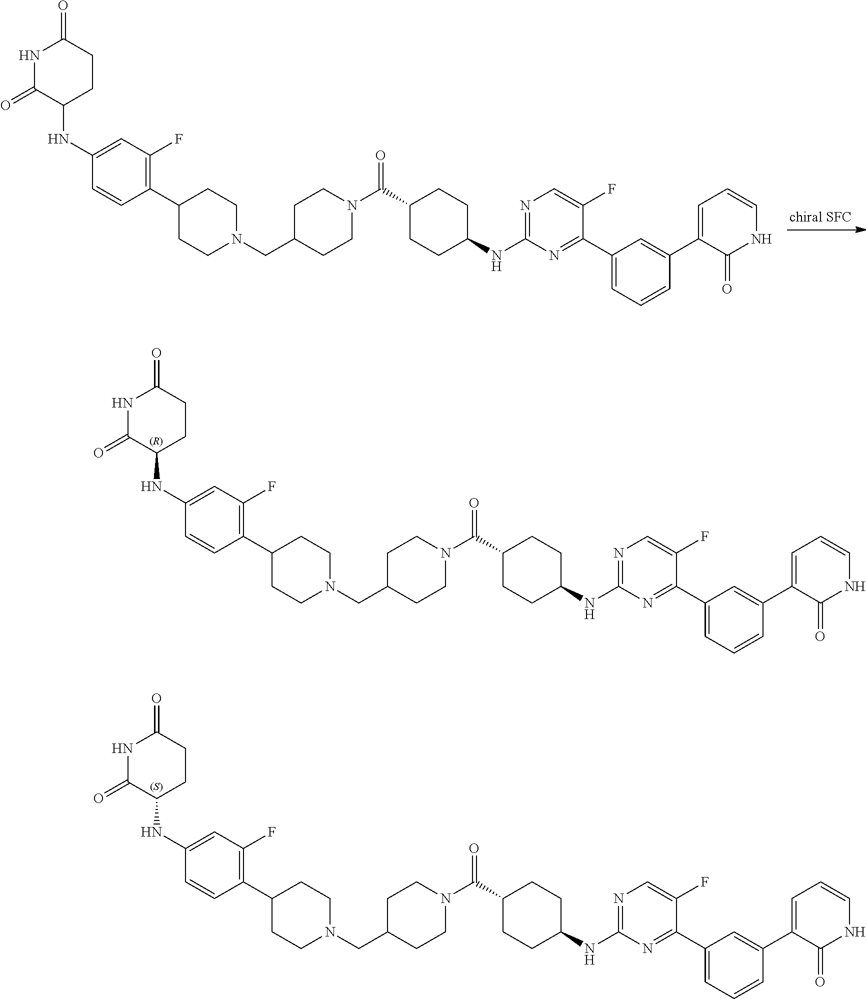

Synthesis of trans-(R)-3-((3-fluoro-4-(1-((1-(-4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperidin-4-yl)phenyl)amino)piperidine-2,6-dione (Compound 421) and trans-(S)-3-((3-fluoro-4-(1-((1-(-4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperidin-4-yl)phenyl)amino)piperidine-2,6-dione (Compound 437) (the two isomers were assigned arbitrarily). Trans-3-((3-fluoro-4-(1-((1-(4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperidin-4-yl)phenyl)amino)piperidine-2,6-dione (Compound 384) (200 mg, 250 umol, 1.00 eq) was separated by chiral SFC (column: DAICEL CHIRALCEL OD (250 mm*30 mm, 10 um); mobile phase: [CO₂—IPA (0.1% IPAm)]; B %: 54%, isocratic elution mode) to give the title compound (Compound 421) (12.2 mg, 14.9 μmol, 11.6% yield, 97.1% purity, 100% ee) (retention time: 1.995, peak 1) as a white solid. LCMS (ES⁺): m/z 793.4 [M+H]⁺ and title compound (Compound 437) (16.2 mg, 19.6 μmol, 15.2% yield, 96.1% purity, 97.0% ee) (retention time: 2.803, peak 2) as a white solid. LCMS (ES⁺): m/z 793.3 [M+H]⁺

### Example 440. Synthesis of 3-((3-fluoro-4-(4-((4-(trans-4-((5-fluoro-4-(3-(2-oxo-1,2-dihydropyridin-3-yl)phenyl)pyrimidin-2-yl)amino)cyclohexane-1-carbonyl)piperazin-1-yl)methyl)piperidin-1-yl)phenyl)amino)piperidine-2,6-dione (Compound 430)

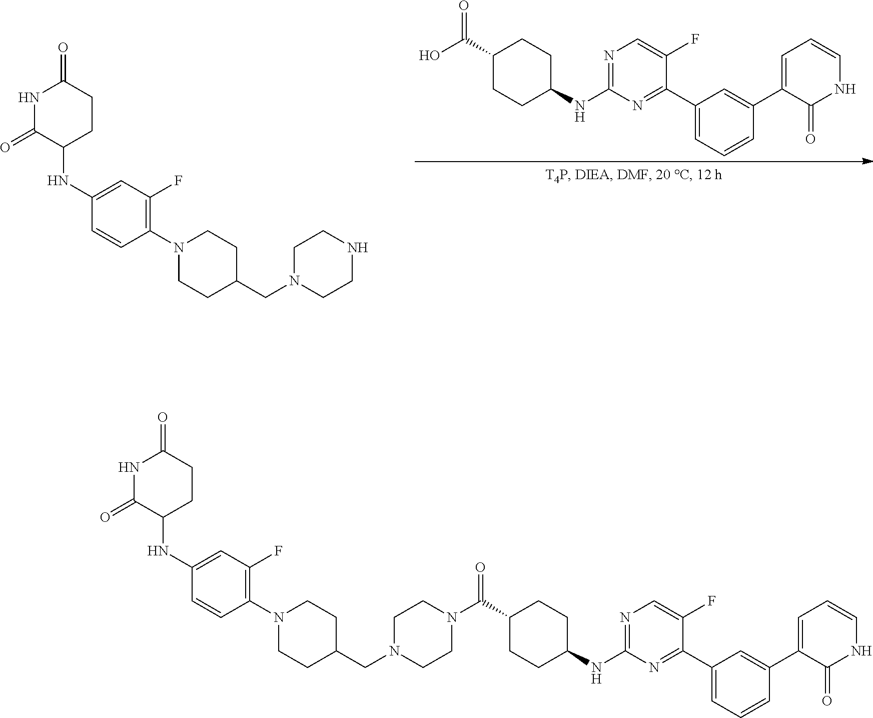 [SMILES: O=C1CCC(Nc2ccc(N3CCC(CN4CCNCC4)CC3)c(F)c2)C(=O)N1 ; O=c1[nH]cccc1-c1cccc(-c2nc(N[C@H]3CC[C@H](C(=O)O)CC3)ncc2F)c1 ; O=C1CCC(Nc2ccc(N3CCC(CN4CCN(C(=O)[C@H]5CC[C@H](Nc6ncc(F)c(-c7cccc(-c8ccc[nH]c8=O)c7)n6)CC5)CC4)CC3)c(F)c2)C(=O)N1]

Synthesis of 3-((3-fluoro-4-(4-((4-(trans-4-((5-fluoro-4-(3-(2-oxo-1,2-dihydropyridin-3-yl)phenyl)pyrimidin-2-yl)amino)cyclohexane-1-carbonyl)piperazin-1-yl)methyl)piperidin-1-yl)phenyl)amino)piperidine-2,6-dione (Compound 430). To a solution of 3-((3-fluoro-4-(4-(piperazin-1-ylmethyl)piperidin-1-yl)phenyl)amino)piperidine-2,6-dione (779 mg, 1.93 mmol, 1.00 eq) in DMF (5 mL) was added trans-4-((5-fluoro-4-(3-(2-oxo-1,2-dihydropyridin-3-yl)phenyl)pyrimidin-2-yl)amino)cyclohexane-1-carboxylic acid (429 mg, 966 μmol, 0.500 eq, HCl), DIEA (499 mg, 3.86 mmol, 673 μL, 2.00 eq) and T₄P (1.39 g, 3.86 mmol, 2.00 eq) at 20 °C. The mixture was stirred at 20 °C for 12 h. LC-MS showed the desired mass was detected. The reaction mixture was filtered and the filtrate concentrated under high vacuum. The residue was purified by prep-HPLC (Waters Xbridge Prep OBD C18 150*40 mm*10 um; mobile phase: [H₂O (10 mM NH₄HCO₃)-ACN]; gradient: 30%-75% B over 8.0 min) to give the title compound (Compound 430) (26.4 mg, 32.9 μmol, 1.71% yield, 99.2% purity) as a white solid. LCMS (ES⁺): m/z 794.4 [M+H]⁺.

### Example 441. Synthesis of trans-3-[4-[3,3-difluoro-1-[[1-[4-[[5-fluoro-4-[3-(2-oxo-1H-pyridin-3-yl)phenyl]pyrimidin-2-yl]amino]cyclohexanecarbonyl]-4-piperidyl]methyl]-4-piperidyl]-3-fluoro-anilino]piperidine-2,6-dione (Compound 408)

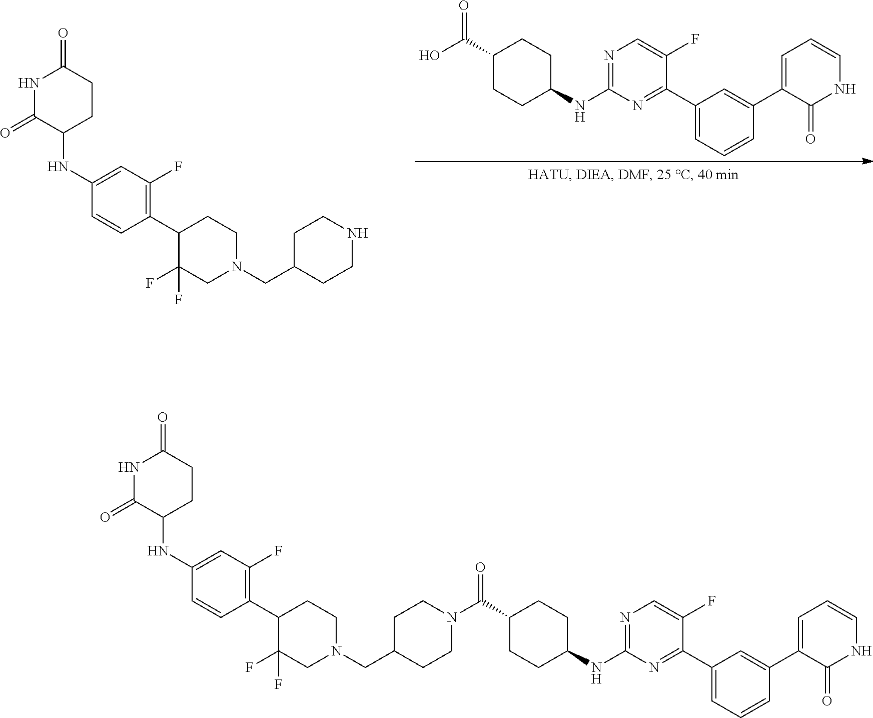 [SMILES: O=C1CCC(Nc2ccc(C3CCN(CC4CCNCC4)CC3(F)F)c(F)c2)C(=O)N1 ; O=c1[nH]cccc1-c1cccc(-c2nc(N[C@H]3CC[C@H](C(=O)O)CC3)ncc2F)c1 ; O=C1CCC(Nc2ccc(C3CCN(CC4CCN(C(=O)[C@H]5CC[C@H](Nc6ncc(F)c(-c7cccc(-c8ccc[nH]c8=O)c7)n6)CC5)CC4)CC3(F)F)c(F)c2)C(=O)N1]

Synthesis of trans-3-[4-[3,3-difluoro-1-[[1-[4-[[5-fluoro-4-[3-(2-oxo-1H-pyridin-3-yl)phenyl]pyrimidin-2-yl]amino]cyclohexanecarbonyl]-4-piperidyl]methyl]-4-piperidyl]-3-fluoro-anilino]piperidine-2,6-dione (Compound 408). To a solution of trans-4-[[5-fluoro-4-[3-(2-oxo-1H-pyridin-3-yl)phenyl]pyrimidin-2-yl]amino]cyclohexanecarboxylic acid (130 mg, 319 μmol, 0.800 eq) in DMF (4 mL) were added DIEA (257 mg, 1.99 mmol, 346 μL, 5.00 eq) and HATU (121 mg, 319 μmol, 0.800 eq) at 25 °C, the mixture was stirred at 25 °C for 10 min. Then 3-[4-[3,3-difluoro-1-(4-piperidylmethyl)-4-piperidyl]-3-fluoro-anilino]piperidine-2,6-dione (220 mg, 398 μmol, 1.00 eq, TFA) was added at 25 °C, the mixture was stirred at 40 °C for 30 min. LCMS showed that the desired mass was detected. To the mixture was added H₂O (5 mL) and the mixture was extracted with EtOAc (3 mL*3). The combined organic layers were washed with brine (5 mL), dried over Na₂SO₄, filtered and the filtrate was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to give the title compound (Compound 408) (35.2 mg, 39.2 μmol, 9.83% yield, 92.2% purity) as a white solid LCMS (ES⁺): m/z 829.4 [M+H]⁺

### Example 442. Synthesis of trans-N-(1′-(4-((2,6-dioxopiperidin-3-yl)amino)-2-fluorophenyl)-3′,3′-difluoro-[1,4′-bipiperidin]-4-yl)-4-((5-fluoro-4-(3-(2-oxo-1,2-dihydropyridin-3-yl)phenyl)pyrimidin-2-yl)amino)cyclohexane-1-carboxamide (Compound 451)

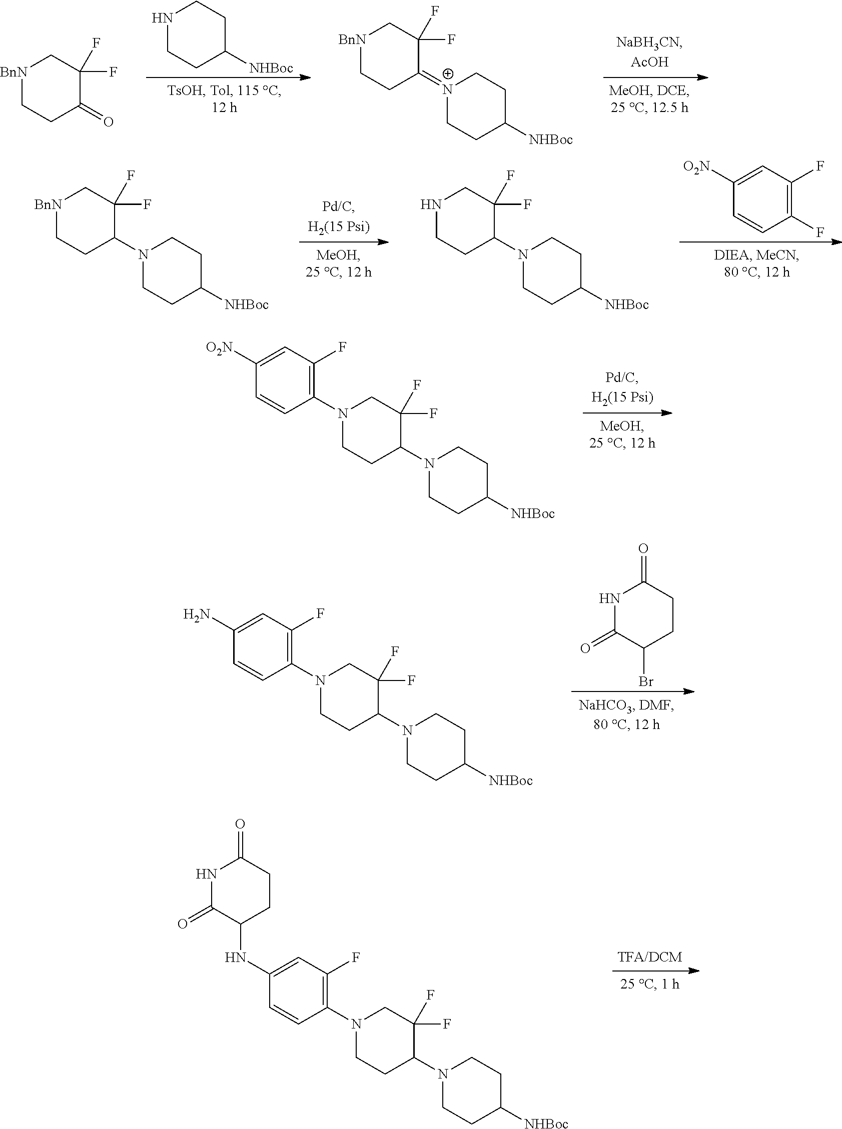

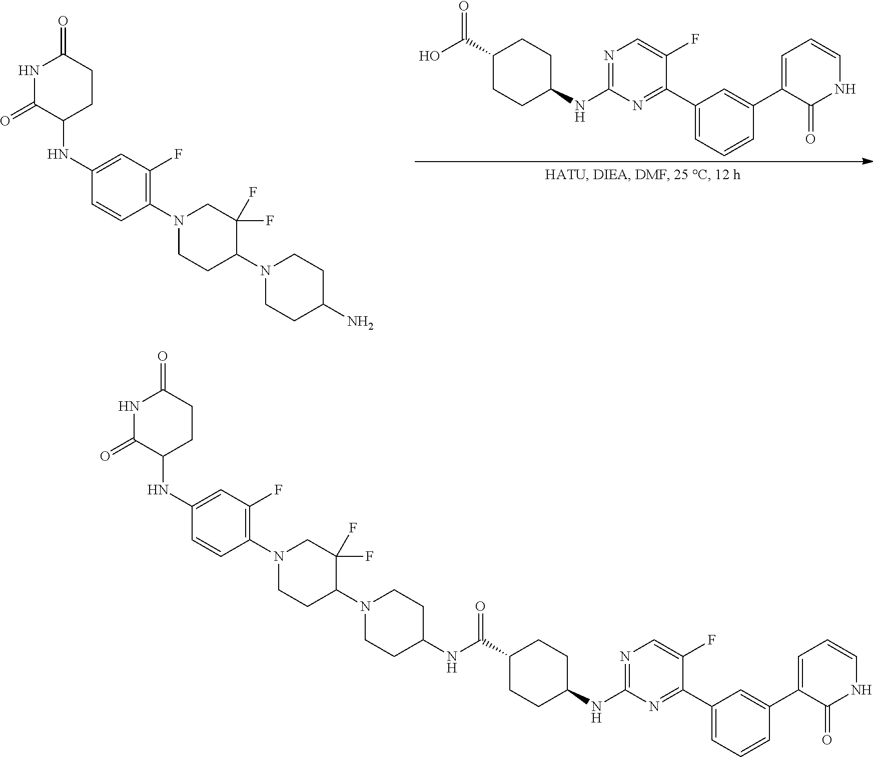 [SMILES: NC1CCN(C2CCN(c3ccc(NC4CCC(=O)NC4=O)cc3F)CC2(F)F)CC1 ; O=c1[nH]cccc1-c1cccc(-c2nc(N[C@H]3CC[C@H](C(=O)O)CC3)ncc2F)c1 ; O=C1CCC(Nc2ccc(N3CCC(N4CCC(NC(=O)[C@H]5CC[C@H](Nc6ncc(F)c(-c7cccc(-c8ccc[nH]c8=O)c7)n6)CC5)CC4)C(F)(F)C3)c(F)c2)C(=O)N1]

Synthesis of 1-(1-benzyl-3,3-difluoropiperidin-4-ylidene)-4-((tert-butoxlycarbonyl)amino)piperidin-1-ium. To a solution of 1-benzyl-3,3-difluoro-piperidin-4-one (5.00 g, 22.2 mmol, 1.00 eq) in Tol. (150 mL) was added tert-butyl N-(4-piperidyl)carbamate (12.0 g, 59.9 mmol, 2.70 eq) and TsOH (1.15 g, 6.66 mmol, 0.300 eq) at 25 °C. The mixture was stirred at 115 °C for 12 h and remove water by Dean-Stark trap. LC-MS showed 1-benzyl-3,3-difluoro-piperidin-4-one was consumed completely and the desired mass was detected. The reaction mixture was filtered and the filter was concentrated to give the title compound (5 g, crude) as a white solid. LCMS (ES⁺): m/z 408.3 [M+H]⁺.

Synthesis of tert-butyl (1′-benzyl-3′,3′-difluoro-[1,4′-bipiperidin]-4-yl)carbamate. To a solution of 1-(1-benzyl-3,3-difluoropiperidin-4-ylidene)-4-((tert-butoxycarbonyl)amino)piperidin-1-ium (5.00 g, 12.2 mmol, 1.00 eq) in MeOH (40 mL), DCE (40 mL) and AcOH (10 mL) at 25 °C. The mixture was stirred at 25 °C for 0.5 h. Then NaBH₃CN (3.85 g, 61.2 mmol, 5.00 eq) was added at 25 °C. The mixture was stirred at 25 °C for 12 h. LC-MS showed 1-(1-benzyl-3,3-difluoropiperidin-4-ylidene)-4-((tert-butoxycarbonyl)amino)piperidin-1-ium was consumed completely and the desired mass was detected. The reaction mixture was diluted with H₂O 50 mL and extracted with EtOAc (50 mL*3). The combined organic layers were concentrated under reduced pressure. The residue was purified by column chromatography (SiO₂, Petroleum ether:Ethyl acetate=0:1 to 1:1) to give the title compound (8 g, crude) as a yellow oil. LCMS (ES⁺): m/z 410.2 [M+H]⁺.

Synthesis of tert-butyl (3′,3′-difluoro-[1,4′-bipiperidin]-4-yl)carbamate. To a solution of tert-butyl (1′-benzyl-3′,3′-difluoro-[1,4′-bipiperidin]-4-yl)carbamate (7.50 g, 18.3 mmol, 1.00 eq) in MeOH (150 mL) was added 10% Pd/C (1.00 g, 4.70 mmol, 50% purity, 2.57 eq) under N₂. The suspension was degassed under vacuum and purged with H₂ several times. The mixture was stirred under H₂ (15 Psi) at 25 °C for 12 h. LC-MS showed tert-butyl (1′-benzyl-3′,3′-difluoro-[1,4′-bipiperidin]-4-yl)carbamate was consumed completely and the desired mass was detected. The reaction mixture was filtered and the filter was concentrated to give the title compound (8 g, crude) as an off-white solid. LCMS (ES⁺): m/z 320.3 [M+H]⁺.

Synthesis of tert-butyl (3′,3′-difluoro-1′-(2-fluoro-4-nitrophenyl)-[1,4′-bipiperidin]-4-yl)carbamate. The reaction was set up in two batches. To a solution of tert-butyl (3′,3′-difluoro-[1,4′-bipiperidin]-4-yl)carbamate (1.00 g, 3.13 mmol, 1.00 eq) in MeCN (10 mL) was added DIEA (1.21 g, 9.39 mmol, 1.64 mL, 3.00 eq) and 1,2-difluoro-4-nitro-benzene (996 mg, 6.26 mmol, 693 μL, 2.00 eq) at 25 °C. The mixture was stirred at 80 °C for 12 h. One batch in parallel was monitored in random. LC-MS showed tert-butyl (3′,3′-difluoro-[1,4′-bipiperidin]-4-yl)carbamate was consumed completely and the desired mass was detected. The reaction mixture was diluted with H₂O 50 mL and extracted with EtOAc (50 mL*3). The combined organic layers were concentrated under reduced pressure. Two batches in parallel were combined. The residue was purified by column chromatography (SiO₂, Petroleum ether:Ethyl acetate=0:1 to 1:1) to give the title compound (1.20 g, 2.00 mmol, 31.9% yield, 76.4% purity) as a white solid. LCMS (ES⁺): m/z 459.2 [M+H]⁺.

Synthesis of tert-butyl (1′-(4-amino-2-fluorophenyl)-3′,3′-difluoro-[1,4′-bipiperidin]-4-yl)carbamate. To a solution of tert-butyl (3′,3′-difluoro-1′-(2-fluoro-4-nitrophenyl)-[1,4′-bipiperidin]-4-yl)carbamate (1.00 g, 2.18 mmol, 1.00 eq) in MeOH (10 mL) was added 10% Pd/C (428 mg, 2.01 mmol, 50% purity, 9.23 eq) under N₂. The suspension was degassed under vacuum and purged with H₂ several times. The mixture was stirred under H₂ (15 Psi) at 25 °C for 12 h. LC-MS showed tert-butyl (3′,3′-difluoro-1′-(2-fluoro-4-nitrophenyl)-[1,4′-bipiperidin]-4-yl)carbamate was consumed completely and the desired mass was detected. The reaction mixture was filtered and the filter was concentrated to give the title compound (1.20 g, crude) as a gray solid. LCMS (ES⁺): m/z 429.2 [M+H]⁺.

Synthesis of tert-butyl (1′-(4-((2,6-dioxopiperidin-3-yl)amino)-2-fluorophenyl)-3′,3′-difluoro-[1,4′-bipiperidin]-4-yl)carbamate. To a solution of tert-butyl (1′-(4-amino-2-fluorophenyl)-3′,3′-difluoro-[1,4′-bipiperidin]-4-yl)carbamate (1.10 g, 2.57 mmol, 1.00 eq) in DMF (15 mL) was added NaHCO₃ (646 mg, 7.70 mmol, 299 μL, 3.00 eq) and 3-bromopiperidine-2,6-dione (1.48 g, 7.70 mmol, 3.00 eq) at 25 °C. The mixture was stirred at 80 °C for 12 h. LC-MS showed tert-butyl (1′-(4-amino-2-fluorophenyl)-3′,3′-difluoro-[1,4′-bipiperidin]-4-yl)carbamate was consumed completely and the desired mass was detected. The reaction mixture was concentrated under reduced pressure. The residue was purified by column chromatography (SiO₂, Petroleum ether:THF=1:0 to 0:1) to give the title compound (760 mg, 1.30 mmol, 50.74% yield, 92.48% purity) as a green oil. LCMS (ES⁺): m/z 540.3 [M+H]⁺.

Synthesis of 3-((4-(4-amino-3′,3′-difluoro-[1,4′-bipiperidin]-1′-yl)-3-fluorophenyl)amino)piperidine-2,6-dione. To a solution of tert-butyl (1′-(4-((2,6-dioxopiperidin-3-yl)amino)-2-fluorophenyl)-3′,3′-difluoro-[1,4′-bipiperidin]-4-yl)carbamate (600 mg, 1.11 mmol, 1.00 eq) in DCM (6 mL) was added TFA (3.07 g, 26.9 mmol, 2.00 mL, 24.2 eq) at 25 °C. The mixture was stirred at 25 °C for 1 h. LC-MS showed tert-butyl (1′-(4-((2,6-dioxopiperidin-3-yl)amino)-2-fluorophenyl)-3′,3′-difluoro-[1,4′-bipiperidin]-4-yl)carbamate was consumed completely and the desired mass was detected. The reaction was concentrated under reduced pressure to give the title compound (700 mg, crude, TFA) as a brown oil. LCMS (ES⁺): m/z 440.2 [M+H]⁺.

Synthesis of trans-N-(1′-(4-((2,6-dioxopiperidin-3-yl)amino)-2-fluorophenyl)-3′,3′-difluoro-[1,4′-bipiperidin]-4-yl)-4-((5-fluoro-4-(3-(2-oxo-1,2-dihydropyridin-3-yl)phenyl)pyrimidin-2-yl)amino)cyclohexane-1-carboxamide (Compound 451). To a solution of 3-((4-(4-amino-3′,3′-difluoro-[1,4′-bipiperidin]-1′-yl)-3-fluorophenyl)amino)piperidine-2,6-dione (250 mg, 451 μmol, 1.00 eq, TFA) in DMF (2 mL) was added HATU (171 mg, 451 mol, 1.00 eq), trans-4-((5-fluoro-4-(3-(2-oxo-1,2-dihydropyridin-3-yl)phenyl)pyrimidin-2-yl)amino)cyclohexane-1-carboxylic acid (184 mg, 451 μmol, 1.00 eq) and DIEA (175 mg, 1.36 mmol, 236 μL, 3.00 eq) at 25 °C. The mixture was stirred at 25 °C for 12 h. LC-MS showed 3-((4-(4-amino-3′,3′-difluoro-[1,4′-bipiperidin]-1′-yl)-3-fluorophenyl)amino)piperidine-2,6-dione was consumed completely and the desired mass was detected. The reaction mixture was concentrated under reduced pressure. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to give desired compound as a white solid. The residue was purified by prep-HPLC (column: Phenomenex luna C18 100*40 mm*3 um; mobile phase: [H₂O (0.2% FA)-ACN]; gradient: 1%-35% B over 8.0 min) to give the title compound (Compound 451) (58.1 mg, 67.0 μmol, 14.8% yield, 95.8% purity) as a pale yellow solid. LCMS (ES⁺): m/z 830.4 [M+H]⁺.

### Example 443. Synthesis of trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(2-oxo-1,2-dihydropyridin-3-yl)phenyl)pyrimidin-2-yl)amino)cyclohexane-1-carboxamide (Compound 453)

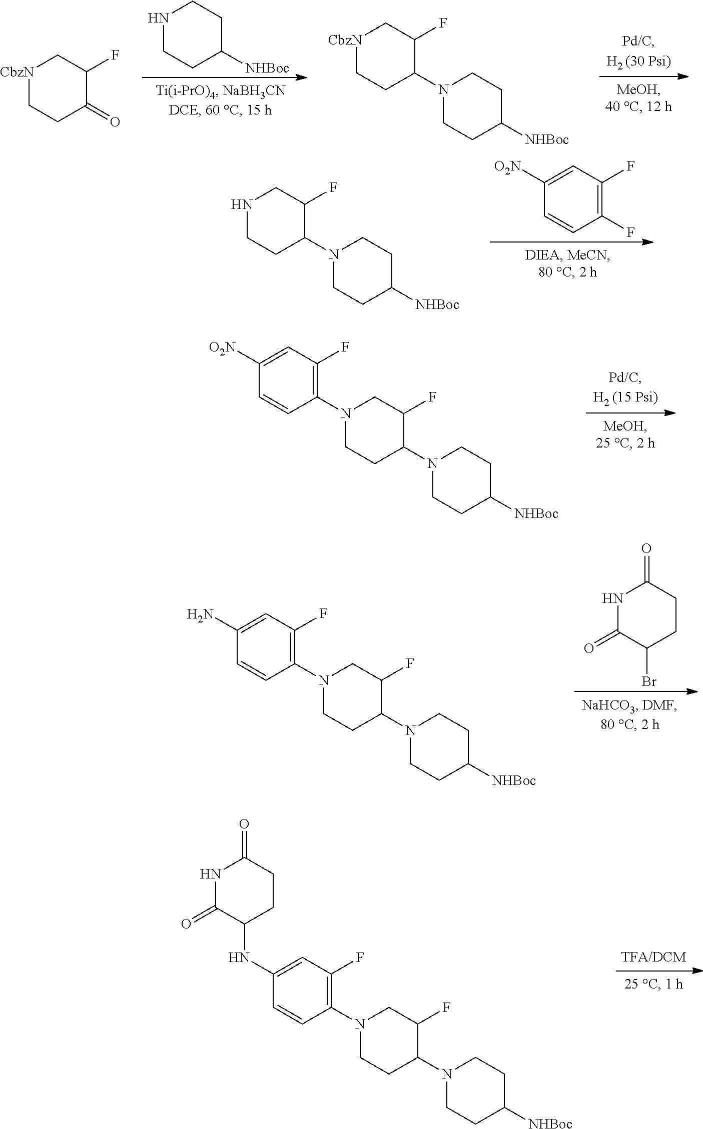

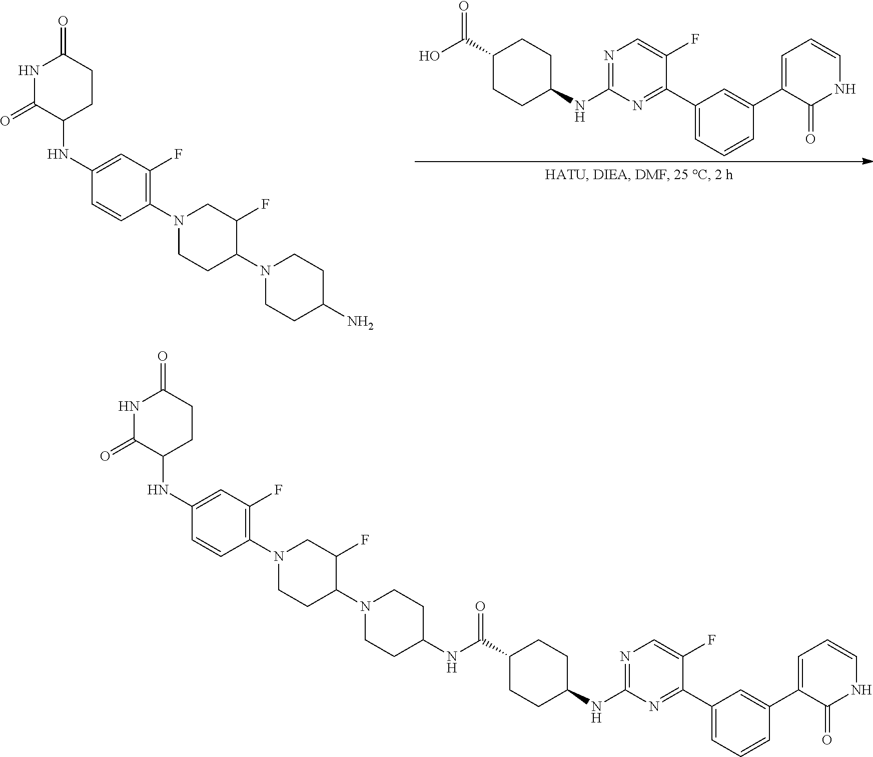 [SMILES: NC1CCN(C2CCN(c3ccc(NC4CCC(=O)NC4=O)cc3F)CC2F)CC1 ; O=c1[nH]cccc1-c1cccc(-c2nc(N[C@H]3CC[C@H](C(=O)O)CC3)ncc2F)c1 ; O=C1CCC(Nc2ccc(N3CCC(N4CCC(NC(=O)[C@H]5CC[C@H](Nc6ncc(F)c(-c7cccc(-c8ccc[nH]c8=O)c7)n6)CC5)CC4)C(F)C3)c(F)c2)C(=O)N1]

Synthesis of benzyl 4-((tert-butoxycarbonyl)amino)-3′-fluoro-[1,4′-bipiperidine]-1′-carboxylate. To a solution of benzyl 3-fluoro-4-oxopiperidine-1-carboxylate (15.0 g, 59.7 mmol, 1.00 eq) in DCE (100 mL) was added Ti(i-PrO)₄ (16.9 g, 59.0 mmol, 17.6 mL, 1.00 eq) and tert-butyl N-(4-piperidyl)carbamate (17.9 g, 89.5 mmol, 1.50 eq) at 25 °C, the mixture was stirred at 60 °C for 12 h, and add NaBH₃CN (11.2 g, 179 mmol, 3.00 eq) at 25 °C. The mixture was stirred at 60 °C for 3 h. LC-MS showed benzyl 3-fluoro-4-oxopiperidine-1-carboxylate was consumed completely and the desired mass was detected. The reaction mixture was quenched by addition H₂O 50 mL at 25 °C, and then combined organic layers were extracted with DCM (90 mL*3), and concentrated under reduced pressure. The residue was purified by column chromatography (SiO₂, Dichloromethane:Methanol=1:1 to 5:1) to give the title compound (18.0 g, 31.4 mmol, 52.6% yield, 76.0% purity) as a yellow oil. LCMS (ES⁺): m/z 436.3 [M+H]⁺.

Synthesis of tert-butyl (3′-fluoro-[1,4′-bipiperidin]-4-yl)carbamate. To a solution of benzyl 4-((tert-butoxycarbonyl)amino)-3′-fluoro-[1,4′-bipiperidine]-1′-carboxylate (6.00 g, 13.7 mmol, 1.00 eq) in MeOH (50 mL) was added 10% Pd/C (2.05 g, 9.64 mmol, 50.0% purity, 0.700 eq) under N₂ atmosphere. The suspension was degassed and purged with H₂ for 3 times. The mixture was stirred under H₂ (30 Psi) at 40 °C for 12 h. LC-MS showed benzyl 4-((tert-butoxycarbonyl)amino)-3′-fluoro-[1,4′-bipiperidine]-1′-carboxylate was consumed completely and the desired mass was detected. The reaction mixture was filtered and concentrated under reduced pressure to give the title compound (4.00 g, crude) as a white solid. LCMS (ES⁺): m/z 302.3 [M+H]⁺.

Synthesis of tert-butyl (3′-fluoro-1′-(2-fluoro-4-nitrophenyl)-[1,4′-bipiperidin]-4-yl)carbamate. To a solution of tert-butyl (3′-fluoro-[1,4′-bipiperidin]-4-yl)carbamate (3.50 g, 11.6 mmol, 1.00 eq) in MeCN (30 mL) was added DIEA (2.25 g, 17.4 mmol, 3.00 mL, 1.50 eq) and 1,2-difluoro-4-nitro-benzene (1.85 g, 11.6 mmol, 1.29 mL, 1.00 eq) at 25 °C. The mixture was stirred at 80 °C for 2 h. LC-MS showed tert-butyl (3′-fluoro-[1,4′-bipiperidin]-4-yl)carbamate was consumed completely and the desired mass was detected. The reaction mixture was quenched by addition H₂O 50 mL at 25 °C, and then combined organic layers were extracted with EtOAC (90 mL*3), and concentrated under reduced pressure. The residue was purified by column chromatography (SiO₂, Petroleum ether:Ethyl acetate=5:1 to 1:1) to give the title compound (1.60 g, 3.23 mmol, 27.7% yield, 88.8% purity) as a brown solid. LCMS (ES⁺): m/z 441.3 [M+H]⁺.

Synthesis of tert-butyl (1′-(4-amino-2-fluorophenyl)-3′-fluoro-[1,4′-bipiperidin]-4-yl)carbamate. To a solution of tert-butyl (3′-fluoro-1′-(2-fluoro-4-nitrophenyl)-[1,4′-bipiperidin]-4-yl)carbamate (1.60 g, 3.63 mmol, 1.00 eq) in MeOH (25 mL) was added 10% Pd/C (541 mg, 2.54 mmol, 50.0% purity, 0.700 eq) at 25 °C under N₂ atmosphere. The suspension was degassed and purged with H₂ for 3 times. The mixture was stirred under H₂ (15 Psi) at 25 °C for 2 h. LC-MS showed tert-butyl (3′-fluoro-1′-(2-fluoro-4-nitrophenyl)-[1,4′-bipiperidin]-4-yl)carbamate was consumed completely and the desired mass was detected. The reaction mixture was filtered and concentrated under reduced pressure to give the title compound (1.38 g, crude) as a light yellow solid. LCMS (ES⁺): m/z 411.2 [M+H]⁺.

Synthesis of tert-butyl (1′-(4-((2,6-dioxopiperidin-3-yl)amino)-2-fluorophenyl)-3′-fluoro-[1,4′-bipiperidin]-4-yl)carbamate. To a solution of tert-butyl (1′-(4-amino-2-fluorophenyl)-3′-fluoro-[1,4′-bipiperidin]-4-yl)carbamate (700 mg, 1.71 mmol, 1.00 eq) in DMF (10 mL) was added NaHCO₃ (286 mg, 3.41 mmol, 132 μL, 2.00 eq) and 3-bromopiperidine-2,6-dione (327 mg, 1.71 mmol, 1.00 eq) at 25 °C. The mixture was stirred at 80 °C for 2 h. LC-MS showed tert-butyl (1′-(4-amino-2-fluorophenyl)-3′-fluoro-[1,4′-bipiperidin]-4-yl)carbamate was remained, The desired mass was detected. The reaction mixture was concentrated under reduced pressure. The residue was purified by column chromatography (SiO₂, Petroleum ether:THF=1:1 to 0:1) to give the title compound (800 mg, 1.33 mmol, 77.8% yield, 86.5% purity) as a green oil. LCMS (ES⁺): m/z 522.3 [M+H]⁺.

Synthesis of 3-((4-(4-amino-3′-fluoro-[1,4′-bipiperidin]-1′-yl)-3-fluorophenyl)amino)piperidine-2,6-dione. To a solution of tert-butyl (1′-(4-((2,6-dioxopiperidin-3-yl)amino)-2-fluorophenyl)-3′-fluoro-[1,4′-bipiperidin]-4-yl)carbamate (700 mg, 1.34 mmol, 1.00 eq) in DCM (4 mL) was added TFA (3.07 g, 26.9 mmol, 2.00 mL, 20.0 eq) at 25 °C. The mixture was stirred at 25 °C for 1 h. LC-MS showed tert-butyl (1′-(4-((2,6-dioxopiperidin-3-yl)amino)-2-fluorophenyl)-3′-fluoro-[1,4′-bipiperidin]-4-yl)carbamate was consumed completely and the desired mass was detected. The reaction mixture was filtered and concentrated under reduced pressure to give the title compound (560 mg, crude, TFA) as a black oil. LCMS (ES⁺): m/z 422.2 [M+H]⁺.

Synthesis of trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(2-oxo-1,2-dihydropyridin-3-yl)phenyl)pyrimidin-2-yl)amino)cyclohexane-1-carboxamide (Compound 453). To a solution of 3-((4-(4-amino-3′-fluoro-[1,4′-bipiperidin]-1′-yl)-3-fluorophenyl)amino)piperidine-2,6-dione (250 mg, 466 μmol, 1.00 eq, TFA), trans-4-((5-fluoro-4-(3-(2-oxo-1,2-dihydropyridin-3-yl)phenyl)pyrimidin-2-yl)amino)cyclohexane-1-carboxylic acid (133 mg, 326 μmol, 0.700 eq) in DMF (5 mL) was added DIEA (181 mg, 1.40 mmol, 243 μL, 3.00 eq) and HATU (266 mg, 700 μmol, 1.50 eq) at 25 °C. The mixture was stirred at 25 °C for 2 h. LC-MS showed the desired mass was detected. The reaction mixture was concentrated under reduced pressure. The residue was purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min) to give the title compound (Compound 453) (87.7 mg, 102 μmol, 21.9% yield, 94.8% purity) as a pale yellow solid. LCMS (ES⁺): m/z 812.4 [M+H]⁺.

### Example 444. Synthesis of trans-3-(6-(1-((1-(4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperidin-4-yl)-1-methyl-1H-indazol-3-yl)piperidine-2,6-dione (Compound 391)

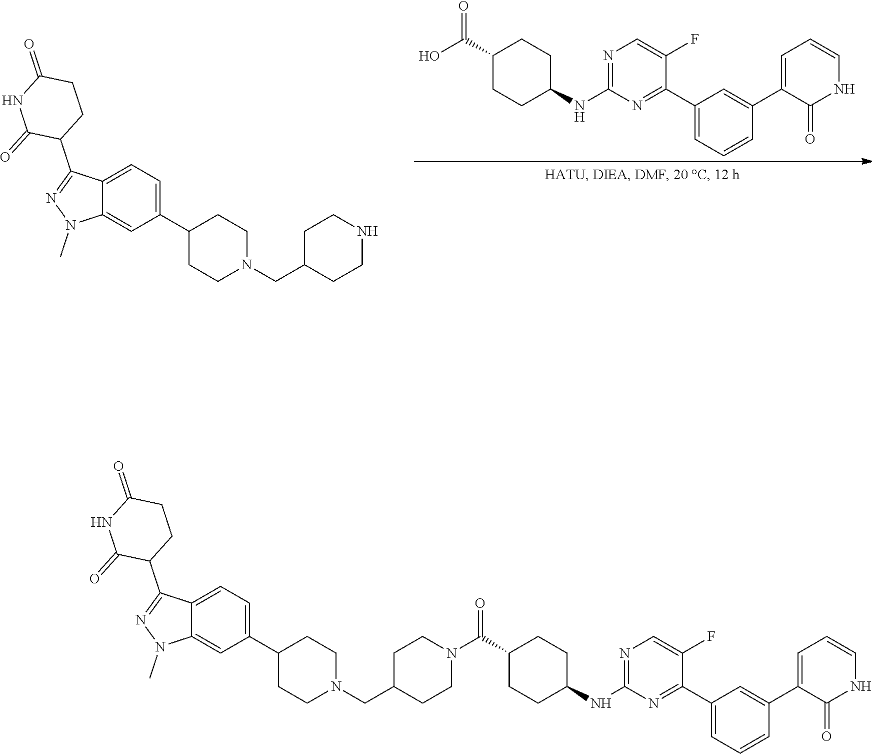 [SMILES: Cn1nc(C2CCC(=O)NC2=O)c2ccc(C3CCN(CC4CCNCC4)CC3)cc21 ; O=c1[nH]cccc1-c1cccc(-c2nc(N[C@H]3CC[C@H](C(=O)O)CC3)ncc2F)c1 ; Cn1nc(C2CCC(=O)NC2=O)c2ccc(C3CCN(CC4CCN(C(=O)[C@H]5CC[C@H](Nc6ncc(F)c(-c7cccc(-c8ccc[nH]c8=O)c7)n6)CC5)CC4)CC3)cc21]

Synthesis of trans-3-(6-(1-((1-(4-((5-fluoro-4-(3-(2-oxo-1,2-dihydropyridin-3-yl)phenyl)pyrimidin-2-yl)amino)cyclohexane-1-carbonyl)piperidin-4-yl)methyl)piperidin-4-yl)-1-methyl-1H-indazol-3-yl)piperidine-2,6-dione (Compound 391). To a solution of trans-4-((5-fluoro-4-(3-(2-oxo-1,2-dihydropyridin-3-yl)phenyl)pyrimidin-2-yl)amino)cyclohexane-1-carboxylic acid (500 mg, 1.22 mmol, 1.00 eq), HATU (559 mg, 1.47 mmol, 1.20 eq) and DIEA (790 mg, 6.13 mmol, 1.07 mL, 5.00 eq) in DMF (10 mL) was added 3-(1-methyl-6-(1-(piperidin-4-ylmethyl)piperidin-4-yl)-1H-indazol-3-yl)piperidine-2,6-dione (658 mg, 1.22 mmol, 1.00 eq, TFA) at 20 °C. The mixture was stirred at 20 °C for 12 h. LCMS showed 3-(1-methyl-6-(1-(piperidin-4-ylmethyl)piperidin-4-yl)-1H-indazol-3-yl)piperidine-2,6-dione was consumed and the desired mass was detected. To the mixture was added H₂O (10 mL) and the mixture was extracted with EtOAc (10 mL*3). The combined organic layers were washed with brine (10 mL*3), dried over Na₂SO₄, filtered and the filtrate was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 30%-60% B over 8.0 min) to give the title compound (Compound 391) (74.4 mg, 90.9 μmol, 7.43% yield, 99.5% purity) as a white solid. LCMS (ES⁺): m/z 814.4 [M+H]⁺

### Example 445. Synthesis of trans-3-((3-fluoro-4-(4-((1-(4-((5-fluoro-4-(3-(2-oxo-1,2-dihydropyridin-3-yl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 584)

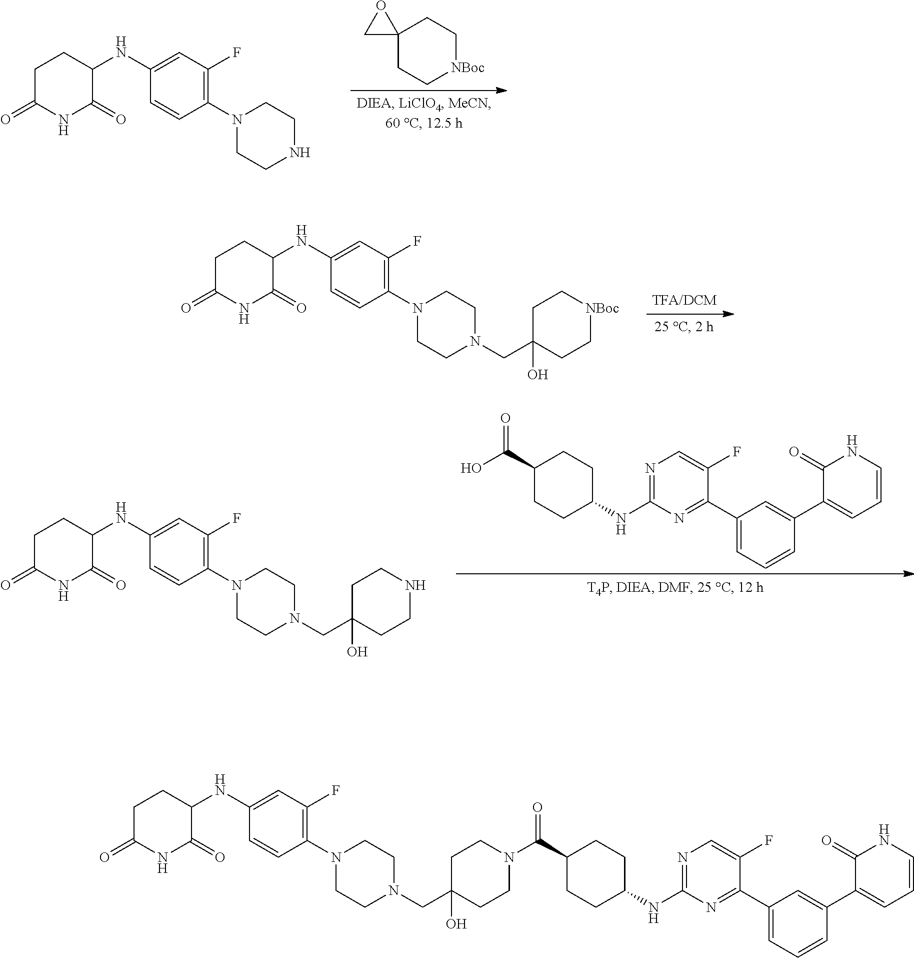 [SMILES: O=C1CCC(Nc2ccc(N3CCNCC3)c(F)c2)C(=O)N1 ; CC(C)(C)OC(=O)N1CCC2(CC1)CO2 ; CC(C)(C)OC(=O)N1CCC(O)(CN2CCN(c3ccc(NC4CCC(=O)NC4=O)cc3F)CC2)CC1 ; O=C1CCC(Nc2ccc(N3CCN(CC4(O)CCNCC4)CC3)c(F)c2)C(=O)N1 ; O=c1[nH]cccc1-c1cccc(-c2nc(N[C@H]3CC[C@H](C(=O)O)CC3)ncc2F)c1 ; O=C1CCC(Nc2ccc(N3CCN(CC4(O)CCN(C(=O)[C@H]5CC[C@H](Nc6ncc(F)c(-c7cccc(-c8ccc[nH]c8=O)c7)n6)CC5)CC4)CC3)c(F)c2)C(=O)N1]

Synthesis of tert-butyl 4-((4-(4-((2,6-dioxopiperidin-3-yl)amino)-2-fluorophenyl)piperazin-1-yl)methyl)-4-hydroxypiperidine-1-carboxylate. To a solution of 3-((3-fluoro-4-(piperazin-1-yl)phenyl)amino)piperidine-2,6-dione (7.00 g, 16.6 mmol, 1.00 eq, TFA) in MeCN (70 mL) was added DIEA (4.30 g, 33.3 mmol, 5.80 mL, 2.00 eq) at 25 °C for 0.5 h, and then add tert-butyl 1-oxa-6-azaspiro[2.5]octane-6-carboxylate (7.10 g, 33.3 mmol, 2.00 eq) at 25 °C, and then add LiClO₄ (5.32 g, 49.9 mmol, 2.20 mL, 3.00 eq) at 25 °C under N₂ atmosphere. The mixture was stirred at 60 °C for 12 h. LC-MS showed 3-((3-fluoro-4-(piperazin-1-yl)phenyl)amino)piperidine-2,6-dione was consumed completely and the desired mass was detected. The reaction mixture was quenched by addition Na₂SO₃ aq. 20 mL at 25 °C, and then diluted with H₂O 10 mL and then extracted with EtOAc (20 mL*3). The combined organic layers were washed with brine (20 mL*3), dried over Na₂SO₄, filtered and concentrated under reduced pressure. The residue was purified by column chromatography (SiO₂, Petroleum ether:Ethyl acetate=1:0 to 3:1) to give the title compound (8.50 g, 10.2 mmol, 61.5% yield, 62.6% purity) as a green solid. LCMS (ES⁺): m/z 520.3 [M+H]⁺.

Synthesis of 3-((3-fluoro-4-(4-((4-hydroxypiperidin-4-yl)methyl)piperazin-1-yl)phenyl)amino)piperidine-2,6-dione. To a solution of tert-butyl 4-((4-(4-((2,6-dioxopiperidin-3-yl)amino)-2-fluorophenyl)piperazin-1-yl)methyl)-4-hydroxypiperidine-1-carboxylate (1.00 g, 1.92 mmol, 1.00 eq) in DCM (8 mL) was added TEA (4.61 g, 40.3 mmol, 3.00 mL, 20.9 eq) at 25 °C. The mixture was stirred at 25 °C for 2 h. LC-MS showed tert-butyl 4-((4-(4-((2,6-dioxopiperidin-3-yl)amino)-2-fluorophenyl)piperazin-1-yl)methyl)-4-hydroxypiperidine-1-carboxylate was consumed completely and the desired mass was detected. The reaction mixture was concentrated under reduced pressure to give the title compound (800 mg, crude, TFA) as a purple solid. LCMS (ES⁺): m/z 420.3 [M+H]⁺.

Synthesis of trans-3-((3-fluoro-4-(4-((1-(4-((5-fluoro-4-(3-(2-oxo-1,2-dihydropyridin-3-yl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 584). To a solution of 3-((3-fluoro-4-(4-((4-hydroxypiperidin-4-yl)methyl)piperazin-1-yl)phenyl)amino)piperidine-2,6-dione (500 mg, 937 μmol, 1.00 eq, TFA) in DMF (5 mL) was added DIEA (363 mg, 2.81 mmol, 489 μL, 3.00 eq), trans-4-((5-fluoro-4-(3-(2-oxo-1,2-dihydropyridin-3-yl)phenyl)pyrimidin-2-yl)amino)cyclohexane-1-carboxylic acid (382 mg, 937 μmol, 1.00 eq) and T₄P (337 mg, 937 μmol, 1.00 eq) at 25 °C. The mixture was stirred at 25 °C for 12 h. LC-MS showed 3-((3-fluoro-4-(4-((4-hydroxypiperidin-4-yl)methyl)piperazin-1-yl)phenyl)amino)piperidine-2,6-dione was consumed completely and the desired mass was detected. The reaction mixture was concentrated under reduced pressure. The residue was purified by prep-HPLC (column: Phenomenex luna C18 100*40 mm*5 um; mobile phase: [H₂O (0.2% FA)-ACN]; gradient: 10%-50% B over 8.0 min) to give the title compound (Compound 584) (73.4 mg, 89.8 μmol, 9.58% yield, 100% purity, 0.16 FA) as a pale yellow solid. LCMS (ES⁺): m/z 810.4 [M+H]⁺.

### Example 446. Synthesis of 3-((3-fluoro-4-(1-((1-(trans-4-((5-fluoro-4-(3-(2-oxo-1,2-dihydropyridin-3-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 581)

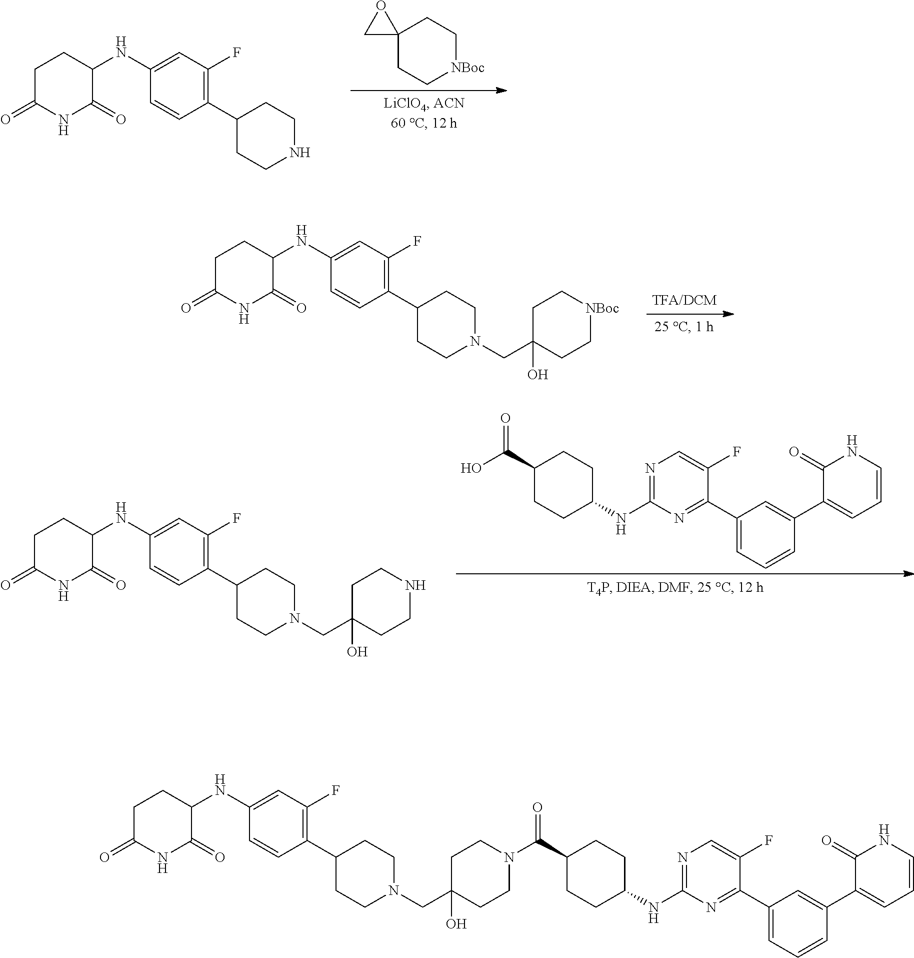 [SMILES: O=C1CCC(Nc2ccc(C3CCNCC3)c(F)c2)C(=O)N1 ; CC(C)(C)OC(=O)N1CCC2(CC1)CO2 ; CC(C)(C)OC(=O)N1CCC(O)(CN2CCC(c3ccc(NC4CCC(=O)NC4=O)cc3F)CC2)CC1 ; O=C1CCC(Nc2ccc(C3CCN(CC4(O)CCNCC4)CC3)c(F)c2)C(=O)N1 ; O=c1[nH]cccc1-c1cccc(-c2nc(N[C@H]3CC[C@H](C(=O)O)CC3)ncc2F)c1 ; O=C1CCC(Nc2ccc(C3CCN(CC4(O)CCN(C(=O)[C@H]5CC[C@H](Nc6ncc(F)c(-c7cccc(-c8ccc[nH]c8=O)c7)n6)CC5)CC4)CC3)c(F)c2)C(=O)N1]

Synthesis of tert-butyl 4-((4-(4-((2,6-dioxopiperidin-3-yl)amino)-2-fluorophenyl)piperidin-1-yl)methyl)-4-hydroxypiperidine-1-carboxylate. To a solution of 3-((3-fluoro-4-(piperidin-4-yl)phenyl)amino)piperidine-2,6-dione (4.00 g, 9.54 mmol, 1.00 eq, TFA) in ACN (30 mL) was added DIEA (3.70 g, 28.6 mmol, 4.98 mL, 3.00 eq) at 25 °C for 1 h, and then add tert-butyl 1-oxa-6-azaspiro[2.5]octane-6-carboxylate (4.07 g, 19.0 mmol, 2.00 eq) at 25 °C, and then add LiClO₄ (3.04 g, 28.6 mmol, 1.26 mL, 3.00 eq) at 25 °C under N₂. The mixture was stirred at 60 °C for 12 h. LC-MS showed the desired mass was detected. The reaction mixture was quenched by addition Na₂SO₃ aq. 20 mL at 25 °C, and then diluted with H₂O 10 mL and then extracted with EtOAc (20 mL*3). The combined organic layers were washed with brine (20 mL*3), dried over Na₂SO₄, filtered and concentrated under reduced pressure. The residue was purified by column chromatography (SiO₂, Petroleum ether:Ethyl acetate=1:0 to 5:1) to give the title compound (4.00 g, 6.48 mmol, 67.9% yield, 84.0% purity) as a white solid. LCMS (ES⁺): m/z 519.2 [M+H]⁺.

Synthesis of 3-((3-fluoro-4-(1-((4-hydroxypiperidin-4-yl)methyl)piperidin-4-yl)phenyl)amino)piperidine-2,6-dione. To a solution of tert-butyl 4-((4-(4-((2,6-dioxopiperidin-3-yl)amino)-2-fluorophenyl)piperidin-1-yl)methyl)-4-hydroxypiperidine-1-carboxylate (3.00 g, 5.78 mmol, 1.00 eq) in DCM (15 mL) was added TFA (7.68 g, 67.3 mmol, 5 mL, 11.6 eq) at 20 °C. The mixture was stirred at 20 °C for 1 h. LC-MS showed the desired mass was detected. The reaction mixture was concentrated under high vacuum to give the title compound (3.00 g, crude, TFA) as a green solid. LCMS (ES⁺): m/z 419.2 [M+H]⁺.

Synthesis of 3-((3-fluoro-4-(1-((1-(trans-4-((5-fluoro-4-(3-(2-oxo-1,2-dihydropyridin-3-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 581). To a solution of 3-((3-fluoro-4-(1-((4-hydroxypiperidin-4-yl)methyl)piperidin-4-yl)phenyl)amino)piperidine-2,6-dione (500 mg, 1.19 mmol, TFA, 1.00 eq) in DMF (3 mL) was added trans-4-((5-fluoro-4-(3-(2-oxo-1,2-dihydropyridin-3-yl)phenyl)pyrimidin-2-yl)amino)cyclohexane-1-carboxylic acid (243 mg, 597 μmol, 0.500 eq), DIEA (308 mg, 2.39 mmol, 416 μL, 2.00 eq) and T4P (860 mg, 2.39 mmol, 2.00 eq) at 20 °C. The mixture was stirred at 20 °C for 12 h. LC-MS showed the desired mass was detected. The reaction mixture was filtered and the filtrate concentrated under high vacuum. The residue was purified by prep-HPLC (Waters Xbridge BEH C18 250*50 mm*10 um; mobile phase: [H₂O (10 mM NH₄HCO₃)-ACN]; gradient: 30%-60% B over 10.0 min) to give the title compound (Compound 581) (91.2 mg, 110.62 μmol, 9.26% yield, 98.12% purity) as a offwhite solid. LCMS (ES⁺): m/z 809.4 [M+H]⁺.

### Example 447. Synthesis of 1′-(4-((2,6-dioxopiperidin-3-yl)amino)-2-fluorophenyl)-3′-fluoro-N-((1r,4r)-4-((5-fluoro-4-(3-(2-oxo-1,2-dihydropyridin-3-yl)phenyl)pyrimidin-2-yl)amino)cyclohexyl)-[1,4′-bipiperidine]-4-carboxamide (Compound 406)

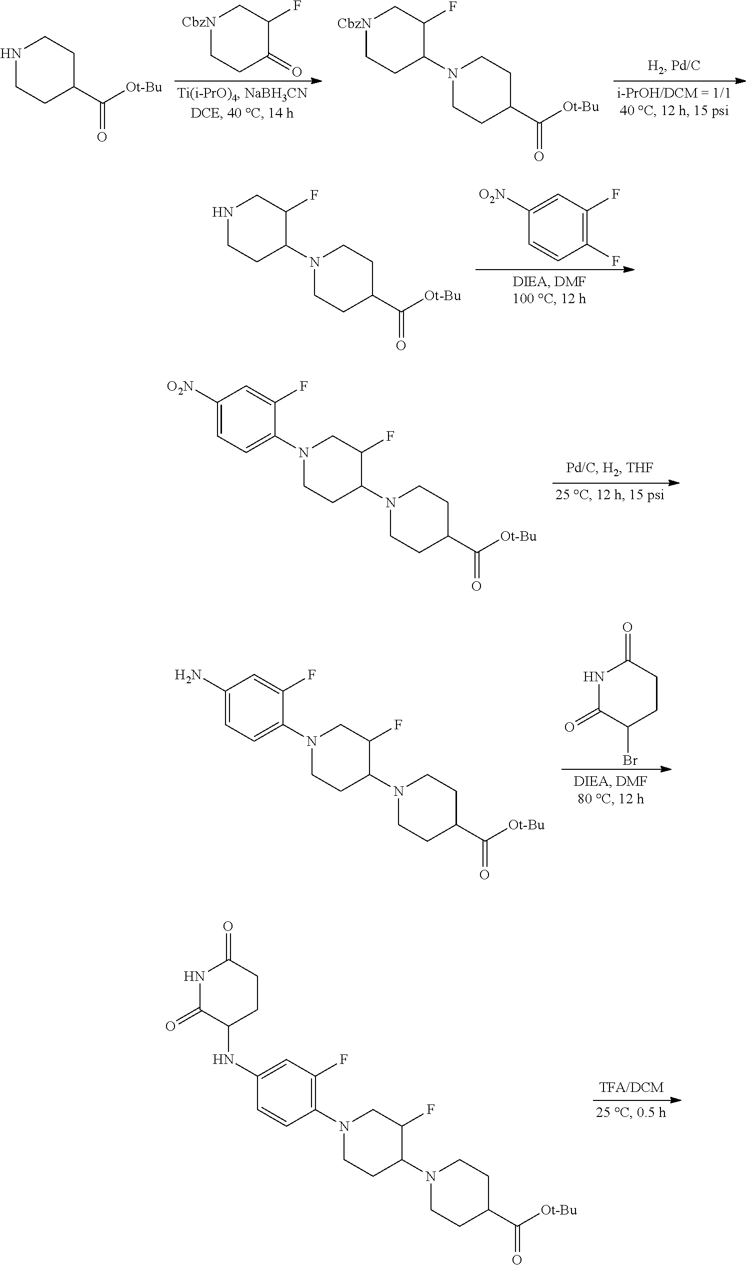

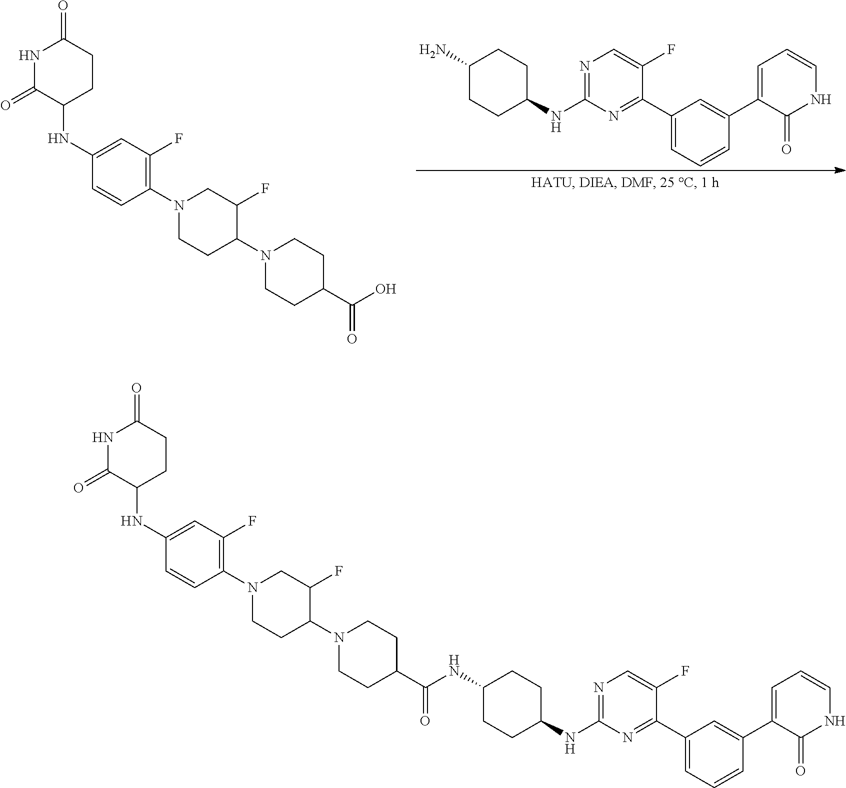 [SMILES: O=C1CCC(Nc2ccc(N3CCC(N4CCC(C(=O)O)CC4)C(F)C3)c(F)c2)C(=O)N1 ; N[C@H]1CC[C@H](Nc2ncc(F)c(-c3cccc(-c4ccc[nH]c4=O)c3)n2)CC1 ; O=C1CCC(Nc2ccc(N3CCC(N4CCC(C(=O)N[C@H]5CC[C@H](Nc6ncc(F)c(-c7cccc(-c8ccc[nH]c8=O)c7)n6)CC5)CC4)C(F)C3)c(F)c2)C(=O)N1]

Synthesis of 1′-benzyl 4-(tert-butyl) 3′-fluoro-[1,4′-bipiperidine]-1,4-dicarboxylate. To a solution of tert-butyl piperidine-4-carboxylate (15.5 g, 83.6 mmol, 1.50 eq) and benzyl 3-fluoro-4-oxopiperidine-1-carboxylate (14.0 g, 55.7 mmol, 1.00 eq) in DCE (300 mL) was added Ti(i-PrO)₄ (31.7 g, 111 mmol, 32.9 mL, 2.00 eq) at 25 °C. The mixture was stirred at 40 °C for 12 h. NaBH₃CN (10.5 g, 167 mmol, 3.00 eq) was added to the mixture at 25 °C. The mixture was stirred at 40 °C for 2 h. LCMS showed the desired mass was detected. The mixture was diluted with H₂O (300 mL) and then extracted with DCM (300 mL*3). The combined organic layers were dried over Na₂SO₄, filtered and the filtrate was concentrated under reduced pressure to give a residue. The residue was purified by MPLC (SiO₂, PE/EtOAc=1/0 to 3/1) to give the title compound (9.00 g, 21.4 mmol, 38.4% yield) as a yellow solid. LCMS (ES⁺): m/z 421.3 [M+H]⁺

Synthesis of tert-butyl 3′-fluoro-[1,4′-bipiperidine]-4-carboxylate. To a solution of 1′-benzyl 4-(tert-butyl) 3′-fluoro-[1,4′-bipiperidine]-1′,4-dicarboxylate (9.00 g, 21.4 mmol, 1.00 eq) in DCM (70 mL) and i-PrOH (70 mL) was added 10% Pd/C (9.00 g, 50% purity) at 25 °C under N₂. The mixture was degassed and purged with H₂ for three times. The mixture was stirred at 40 °C under H₂ (15 psi) for 12 h. LCMS showed 1′-benzyl 4-(tert-butyl) 3′-fluoro-[1,4′-bipiperidine]-1′,4-dicarboxylate was consumed completely and the desired mass was detected. The mixture was filtered and the filtrate was concentrated under reduced pressure to give the title compound (6.00 g, crude) as a white solid. LCMS (ES⁺): m/z 287.3 [M+H]⁺.

Synthesis of tert-butyl 3′-fluoro-1′-(2-fluoro-4-nitrophenyl)-[1,4′-bipiperidine]-4-carboxylate. To a solution of tert-butyl 3′-fluoro-[1,4′-bipiperidine]-4-carboxylate (5.60 g, 19.6 mmol, 1.00 eq) and 1,2-difluoro-4-nitrobenzene (3.11 g, 19.6 mmol, 2.16 mL, 1.00 eq) in DMF (80 mL) was added DIEA (5.05 g, 39.1 mmol, 6.81 mL, 2.00 eq) at 25 °C, the mixture was stirred at 100 °C for 12 h. LCMS showed tert-butyl 3′-fluoro-[1,4′-bipiperidine]-4-carboxylate was consumed completely and the desired mass was detected. The mixture was filtered and the filter cake was dried under reduced pressure to give the title compound (8.30 g, crude) as a yellow solid. LCMS (ES⁺): m/z 426.2 [M+H]⁺.

Synthesis of tert-butyl 1′-(4-amino-2-fluorophenyl)-3′-fluoro-[1,4′-bipiperidine]-4-carboxylate. To a solution of tert-butyl 3′-fluoro-1′-(2-fluoro-4-nitrophenyl)-[1,4′-bipiperidine]-4-carboxylate (8.30 g, 19.5 mmol, 1.00 eq) in THF (160 mL) was added 10% Pd/C (16.0 g, 50% purity) at 25 °C under N₂. The mixture was degassed and purged with H₂ for three times. The mixture was stirred at 25 °C under H₂ (15 psi) for 12 h. LCMS showed tert-butyl 3′-fluoro-1′-(2-fluoro-4-nitrophenyl)-[1,4′-bipiperidine]-4-carboxylate was consumed completely and the desired mass was detected. The mixture was filtered and the filtrate was concentrated under reduced pressure to give the title compound (7.70 g, crude) as a red solid. LCMS (ES⁺): m/z 396.2 [M+H]⁺.

Synthesis of tert-butyl 1′-(4-((2,6-dioxopiperidin-3-yl)amino)-2-fluorophenyl)-3′-fluoro-[1,4′-bipiperidine]-4-carboxylate. To a solution of tert-butyl 1′-(4-amino-2-fluorophenyl)-3′-fluoro-[1,4′-bipiperidine]-4-carboxylate (7.00 g, 17.7 mmol, 1.00 eq) and 3-bromopiperidine-2,6-dione (6.80 g, 35.4 mmol, 2.00 eq) in DMF (100 mL) was added DIEA (4.58 g, 35.4 mmol, 6.17 mL, 2.00 eq) at 25 °C, the mixture was stirred at 80 °C for 12 h. LCMS showed tert-butyl 1′-(4-amino-2-fluorophenyl)-3′-fluoro-[1,4′-bipiperidine]-4-carboxylate was consumed and the desired mass was detected. H₂O (100 mL) was added to the solution, the mixture was extracted with EtOAc (100 mL*3), then the combined organic layers were washed with brine (100 mL*3), dried over Na₂SO₄, then filtered and the filtrate was concentrated under reduced pressure to give a residue. The residue was purified by prep-HPLC (column: Phenomenex luna c18 250 mm*100 mm*10 um; mobile phase: [H₂O (0.2% FA)-ACN]; gradient: 0%-22% B over 20.0 min) to give the title compound (5.00 g, 9.87 mmol, 55.8% yield) as a green solid. LCMS (ES⁺): m/z 507.2 [M+H]⁺.

Synthesis of 1′-(4-((2,6-dioxopiperidin-3-yl)amino)-2-fluorophenyl)-3′-fluoro-[1,4′-bipiperidine]-4-carboxylic acid. A solution of tert-butyl 1′-(4-((2,6-dioxopiperidin-3-yl)amino)-2-fluorophenyl)-3′-fluoro-[1,4′-bipiperidine]-4-carboxylate (1.00 g, 1.97 mmol, 1.00 eq) in DCM (9 mL) and TFA (3 mL) was stirred at 25 °C for 0.5 h. LCMS showed tert-butyl 1′-(4-((2,6-dioxopiperidin-3-yl)amino)-2-fluorophenyl)-3′-fluoro-[1,4′-bipiperidine]-4-carboxylate was consumed and the desired mass was detected. The mixture was concentrated under reduced pressure to give the title compound (1.00 g, crude) as a yellow oil. LCMS (ES⁺): m/z 451.1 [M+H]⁺.

Synthesis of 1′-(4-((2,6-dioxopiperidin-3-yl)amino)-2-fluorophenyl)-3′-fluoro-N-((1r,4r)-4-((5-fluoro-4-(3-(2-oxo-1,2-dihydropyridin-3-yl)phenyl)pyrimidin-2-yl)amino)cyclohexyl)-[1,4′-bipiperidine]-4-carboxamide (Compound 406). To a solution of 1′-(4-((2,6-dioxopiperidin-3-yl)amino)-2-fluorophenyl)-3′-fluoro-[1,4′-bipiperidine]-4-carboxylic acid (240 mg, 533 μmol, 1.00 eq) in DMF (1.5 mL) were added HATU (70.9 mg, 186 μmol, 0.350 eq) and DIEA (345 mg, 2.68 mmol, 465 μL, 5.00 eq) at 25 °C, to the reaction was added trans-3-(3-(2-((4-aminocyclohexyl)amino)-5-fluoropyrimidin-4-yl)phenyl)pyridin-2(1H)-one (222 mg, 533 μmol, 1.00 eq, HCl) at 25 °C, the mixture was stirred at 25 °C for 1 h. LCMS showed 1′-(4-((2,6-dioxopiperidin-3-yl)amino)-2-fluorophenyl)-3′-fluoro-[1,4′-bipiperidine]-4-carboxylic acid was consumed completely and the desired mass was detected. The mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 5%-35% B over 8.0 min) to give the title compound (Compound 406) (66.7 mg, 80.3 μmol, 15.1% yield, 97.7% purity) as a pink solid. LCMS (ES⁺): m/z 812.3 [M+H]⁺

### Example 448. Synthesis of 3-(6-(4-((1-(trans-4-((5-fluoro-4-(3-(2-oxopyridin-1(2H)-yl)phenyl)pyrimidin-2-yl)amino)cyclohexane-1-carbonyl)piperidin-4-yl)methyl)piperazin-1-yl)-1-methyl-1H-indazol-3-yl)piperidine-2,6-dione (Compound 399)

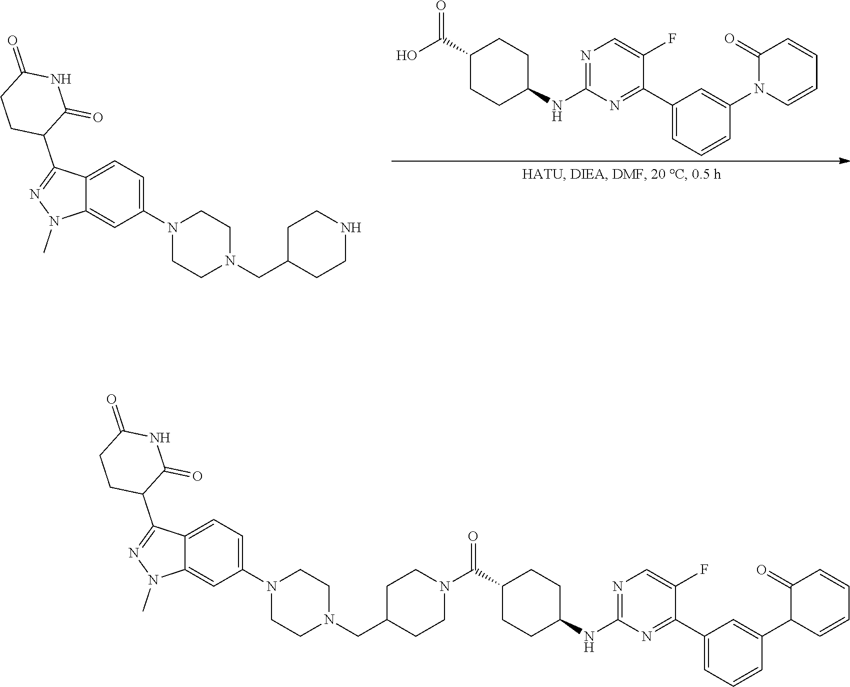 [SMILES: Cn1nc(C2CCC(=O)NC2=O)c2ccc(N3CCN(CC4CCNCC4)CC3)cc21 ; O=c1ccccn1-c1cccc(-c2nc(N[C@H]3CC[C@H](C(=O)O)CC3)ncc2F)c1 ; Cn1nc(C2CCC(=O)NC2=O)c2ccc(N3CCN(CC4CCN(C(=O)[C@H]5CC[C@H](Nc6ncc(F)c(-c7cccc(C8C=CC=CC8=O)c7)n6)CC5)CC4)CC3)cc21]

Synthesis of 3-(6-(4-((1-(trans-4-((5-fluoro-4-(3-(2-oxopyridin-1(2H)-yl)phenyl)pyrimidin-2-yl)amino)cyclohexane-1-carbonyl)piperidin-4-yl)methyl)piperazin-1-yl)-1-methyl-1H-indazol-3-yl)piperidine-2,6-dione (Compound 399). To a solution of 3-(1-methyl-6-(4-(piperidin-4-ylmethyl)piperazin-1-yl)-1H-indazol-3-yl)piperidine-2,6-dione (800 mg, 1.49 mmol, 1.00 eq, TFA) and trans-4-((5-fluoro-4-(3-(2-oxopyridin-1(2H)-yl)phenyl)pyrimidin-2-yl)amino)cyclohexane-1-carboxylic acid (424 mg, 1.04 mmol, 0.700 eq) in DMF (8 mL) was added DIEA (383 mg, 2.97 mmol, 517 μL, 2.00 eq) and HATU (1.13 g, 2.97 mmol, 2.00 eq) at 20 °C. The mixture was stirred at 20 °C for 0.5 h. LC-MS showed the desired mass was detected. The reaction mixture was filtered. The residue was purified by prep-HPLC (column: Phenomenex luna C18 100*40 mm*3 um; mobile phase: [H₂O (0.2% FA)-ACN]; gradient: 5%-40% B over 8.0 min) to give the title compound (Compound 399) (188.7 mg, 229 μmol, 15.4% yield, 100% purity, 0.15 FA) as a light yellow solid. LCMS (ES⁺): m/z 815.4 [M+H]⁺.

### Example 449. Synthesis of trans-3-((3-fluoro-4-(4-((4-(4-((5-fluoro-4-(3-(2-oxopyridin-1(2H)-yl)phenyl)pyrimidin-2-yl)amino)cyclohexane-1-carbonyl)piperazin-1-yl)methyl)piperidin-1-yl)phenyl)amino)piperidine-2,6-dione (Compound 387)

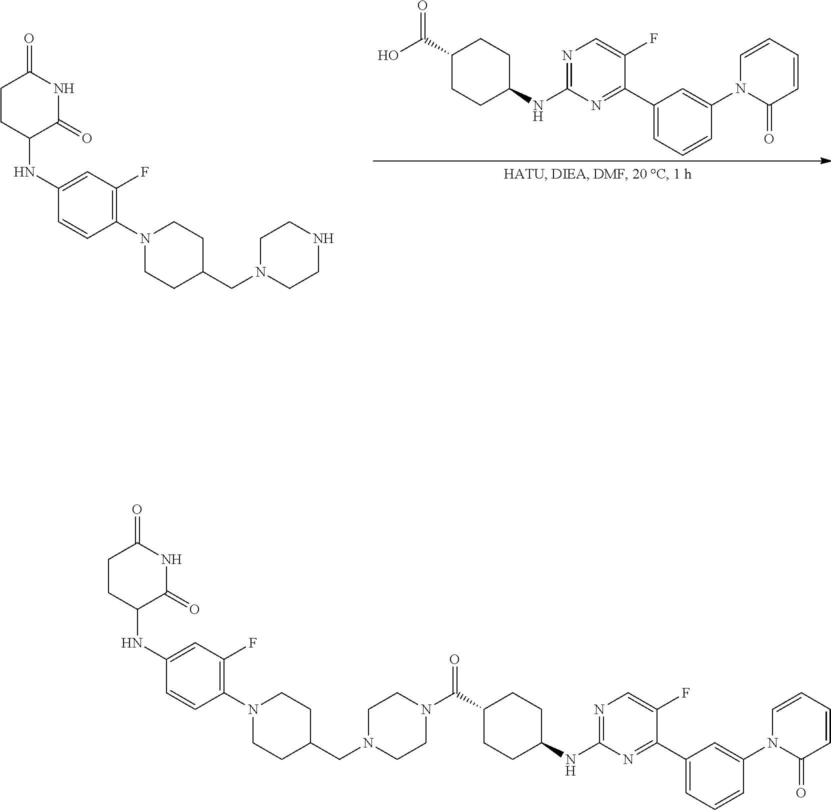 [SMILES: O=C1CCC(Nc2ccc(N3CCC(CN4CCNCC4)CC3)c(F)c2)C(=O)N1 ; O=c1ccccn1-c1cccc(-c2nc(N[C@H]3CC[C@H](C(=O)O)CC3)ncc2F)c1 ; O=C1CCC(Nc2ccc(N3CCC(CN4CCN(C(=O)[C@H]5CC[C@H](Nc6ncc(F)c(-c7cccc(-n8ccccc8=O)c7)n6)CC5)CC4)CC3)c(F)c2)C(=O)N1]

Synthesis of trans-3-((3-fluoro-4-(4-((4-(4-((5-fluoro-4-(3-(2-oxopyridin-1(2H)-yl)phenyl)pyrimidin-2-yl)amino)cyclohexane-1-carbonyl)piperazin-1-yl)methyl)piperidin-1-yl)phenyl)amino)piperidine-2,6-dione (Compound 387). To a solution of 3-[3-fluoro-4-[4-(piperazin-1-ylmethyl)-1-piperidyl]anilino]piperidine-2,6-dione (300 mg, 579 μmol, 1.00 eq, TFA) in DMF (2 mL) was added trans-4-((5-fluoro-4-(3-(2-oxopyridin-1(2H)-yl)phenyl)pyrimidin-2-yl)amino)cyclohexane-1-carboxylic acid (260 mg, 637 μmol, 1.10 eq), DIEA (224 mg, 1.74 mmol, 302 μL, 3.00 eq) and HATU (440 mg, 1.16 mmol, 2.00 eq). The mixture was stirred at 20 °C for 1 h. LC-MS showed the desired mass was detected. The reaction mixture was quenched by addition H₂O 10 mL, and then extracted with EtOAc (10 mL*3). The combined organic layers were dried over Na₂SO₄, filtered and concentrated under reduced pressure. The residue was purified by prep-HPLC (column: Phenomenex luna C18 100*40 mm*3 um; mobile phase: [H₂O (0.2% FA)-ACN]; gradient: 1%-30% B over 8.0 min) to give the title compound (Compound 387) (59.5 mg, 71.5 μmol, 12.3% yield, 95.4% purity) as a grey solid. LCMS (ES⁺): m/z 794.4 [M+H]⁺.

### Example 450. Synthesis of trans-3-[4-[3,3-difluoro-1-[[1-[4-[[5-fluoro-4-[3-(2-oxo-1-pyridyl)phenyl]pyrimidin-2-yl]amino]cyclohexanecarbonyl]-4-piperidyl]methyl]-4-piperidyl]-3-fluoro-anilino]piperidine-2,6-dione (Compound 390)

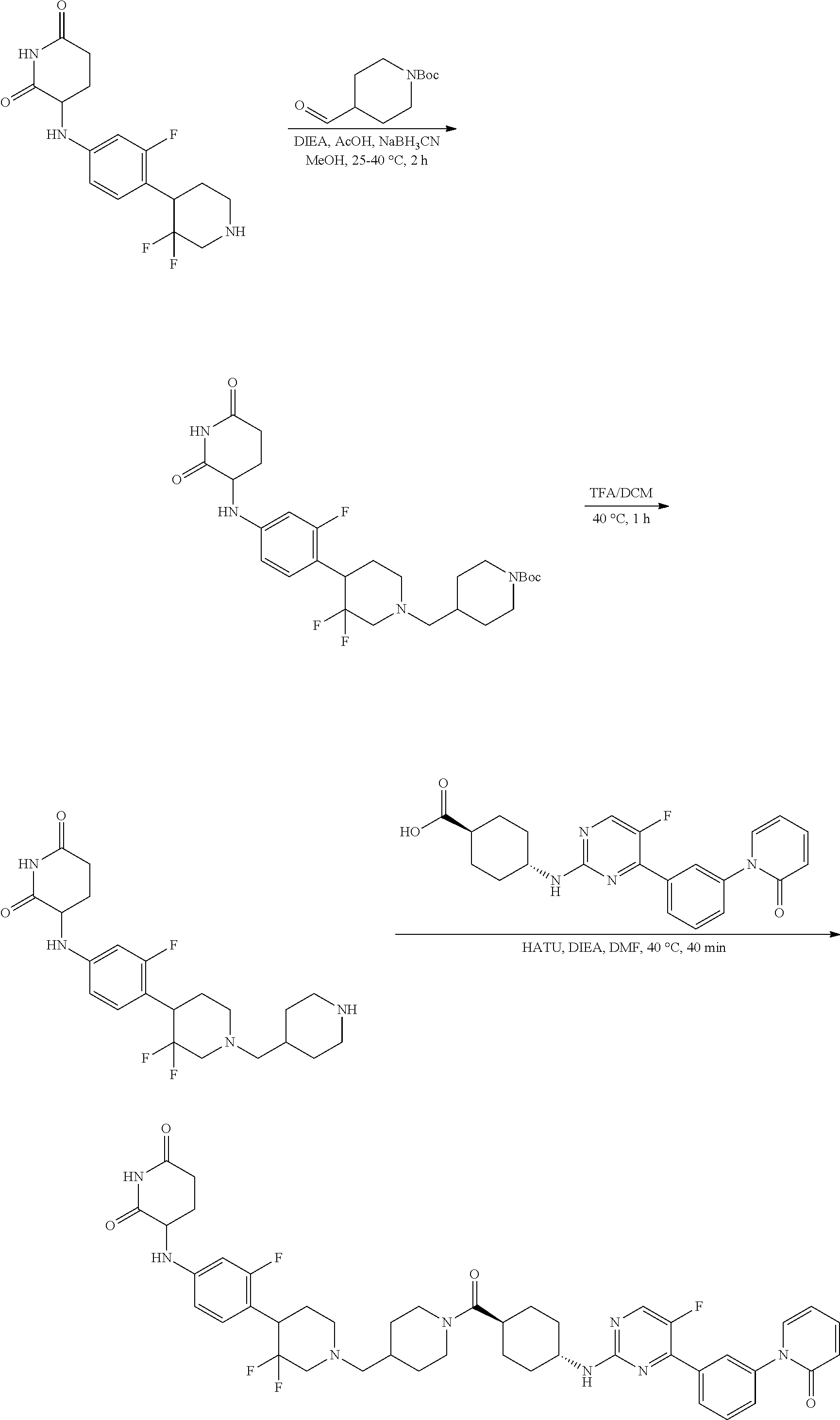 [SMILES: O=C1CCC(Nc2ccc(C3CCNCC3(F)F)c(F)c2)C(=O)N1 ; CC(C)(C)OC(=O)N1CCC(C=O)CC1 ; CC(C)(C)OC(=O)N1CCC(CN2CCC(c3ccc(NC4CCC(=O)NC4=O)cc3F)C(F)(F)C2)CC1 ; O=C1CCC(Nc2ccc(C3CCN(CC4CCNCC4)CC3(F)F)c(F)c2)C(=O)N1 ; O=c1ccccn1-c1cccc(-c2nc(N[C@H]3CC[C@H](C(=O)O)CC3)ncc2F)c1 ; O=C1CCC(Nc2ccc(C3CCN(CC4CCN(C(=O)[C@H]5CC[C@H](Nc6ncc(F)c(-c7cccc(-n8ccccc8=O)c7)n6)CC5)CC4)CC3(F)F)c(F)c2)C(=O)N1]

Synthesis of tert-butyl 4-[[4-[4-[(2,6-dioxo-3-piperidyl)amino]-2-fluoro-phenyl]-3,3-difluoro-1-piperidyl]methyl]piperidine-1-carboxylate. To a solution of 3-[4-(3,3-difluoro-4-piperidyl)-3-fluoro-anilino]piperidine-2,6-dione (400 mg, 878 μmol, 1.00 eq, TFA) in MeOH (5 mL) was added DIEA to adjust pH to 8 at 25 °C, the mixture was stirred at 25 °C for 0.5 h. Then AcOH (52.8 mg, 878 μmol, 50.3 μL, 1.00 eq) and tert-butyl 4-formylpiperidine-1-carboxylate (187 mg, 878 μmol, 1.00 eq) was added to the mixture at 25 °C, pH was 5 now, the mixture was stirred at 40 °C for 0.5 h. Then NaBH₃CN (82.8 mg, 1.32 mmol, 1.50 eq) was added at 25 °C, the mixture was stirred at 40 °C for 1 h. LCMS showed that the desired mass was detected. The mixture was concentrated under reduced to give a residue. To the residue was added H₂O (10 mL) and it was extracted with EtOAc (10 mL*3). The combined organic layers were washed with brine (10 mL*3), dried over Na₂SO₄, filtered and the filtrate was concentrated under reduced pressure to give the title compound (500 mg, crude) as a yellow oil liquid. LCMS (ES⁺): m/z 483.2 [M+H−56]⁺.

Synthesis of 3-[4-[3,3-difluoro-1-(4-piperidylmethyl)-4-piperidyl]-3-fluoro-anilino]piperidine-2,6-dione. To a solution of tert-butyl 4-[[4-[4-[(2,6-dioxo-3-piperidyl)amino]-2-fluoro-phenyl]-3,3-difluoro-1-piperidyl]methyl]piperidine-1-carboxylate (430 mg, 798 μmol, 1.00 eq) in DCM (4 mL) was added TFA (1 mL) at 25 °C, the mixture was stirred at 40 °C for 1 h. LCMS showed that the desired mass was detected. The mixture was concentrated under reduced pressure to give the title compound (440 mg, crude, TFA) as a yellow oil liquid. LCMS (ES⁺): m/z 439.3 [M+H]⁺.

Synthesis of trans-3-[4-[3,3-difluoro-1-[[1-[4-[[5-fluoro-4-[3-(2-oxo-1-pyridyl)phenyl]pyrimidin-2-yl]amino]cyclohexanecarbonyl]-4-piperidyl]methyl]-4-piperidyl]-3-fluoro-anilino]piperidine-2,6-dione (Compound 390). To a solution of trans-4-[[5-fluoro-4-[3-(2-oxo-1-pyridyl)phenyl]pyrimidin-2-yl]amino]cyclohexanecarboxylic acid (214 mg, 525 μmol, 1.00 eq) in DMF (4 mL) were added DIEA (272 mg, 2.10 mmol, 366 μL, 4.00 eq) and HATU (299 mg, 787 μmol, 1.50 eq) at 25 °C, the mixture was stirred at 25 °C for 10 min. Then 3-[4-[3,3-difluoro-1-(4-piperidylmethyl)-4-piperidyl]-3-fluoro-anilino]piperidine-2,6-dione (290 mg, 525 μmol, 1.00 eq, TFA) was added at 25 °C, the mixture was stirred at 40 °C for 30 min. LCMS showed that the desired mass was detected. To the mixture was added H₂O (5 mL) and the mixture was extracted with EtOAc (3 mL*3). The combined organic layers were washed with brine (5 mL), dried over Na₂SO₄, filtered and the filtrate was concentrated under reduced pressure to give a residue. The residue was purified by prep-HPLC (neutral) (column: Phenomenex Gemini-NX 80*40 mm*3 um; mobile phase: [H₂O (10 mM NH₄HCO₃)-ACN]; gradient: 30%-60% B over 8.0 min) to give the title compound (Compound 390) (82.8 mg, 95.6 μmol, 18.2% yield, 95.7% purity) as a white solid. LCMS (ES⁺): m/z 829.3 [M+H]⁺

### Example 451. Synthesis of trans-N-(1′-(4-((2,6-dioxopiperidin-3-yl)amino)-2-fluorophenyl)-3′,3′-difluoro-[1,4′-bipiperidin]-4-yl)-4-((5-fluoro-4-(3-(2-oxopyridin-1(2H)-yl)phenyl)pyrimidin-2-yl)amino)cyclohexane-1-carboxamide (Compound 431)

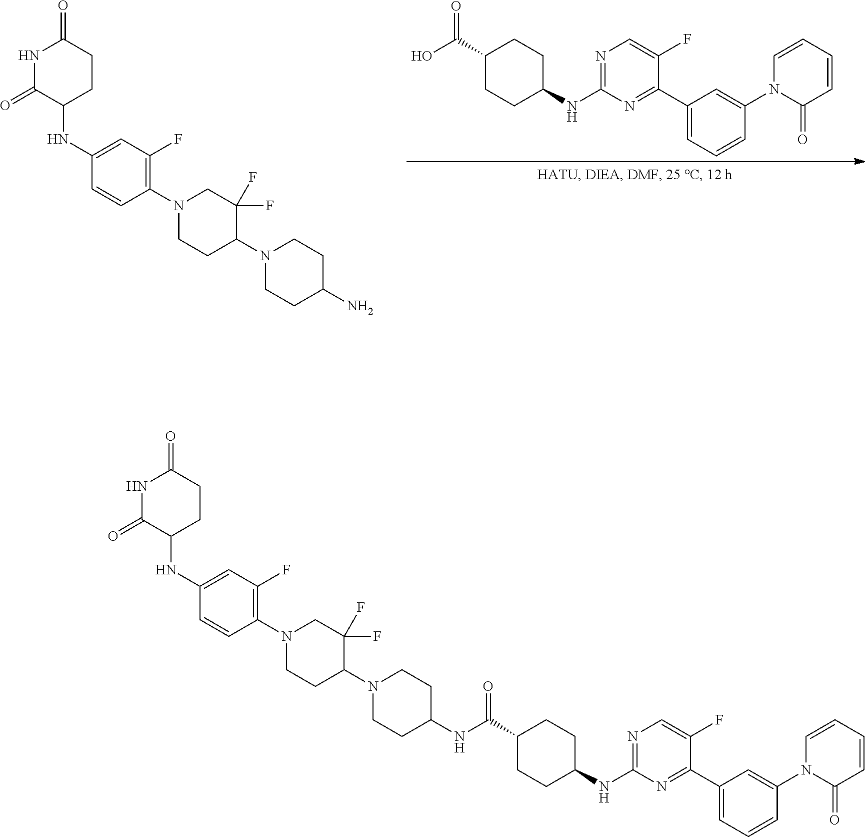 [SMILES: NC1CCN(C2CCN(c3ccc(NC4CCC(=O)NC4=O)cc3F)CC2(F)F)CC1 ; O=c1ccccn1-c1cccc(-c2nc(N[C@H]3CC[C@H](C(=O)O)CC3)ncc2F)c1 ; O=C1CCC(Nc2ccc(N3CCC(N4CCC(NC(=O)[C@H]5CC[C@H](Nc6ncc(F)c(-c7cccc(-n8ccccc8=O)c7)n6)CC5)CC4)C(F)(F)C3)c(F)c2)C(=O)N1]

Synthesis of trans-N-(1′-(4-((2,6-dioxopiperidin-3-yl)amino)-2-fluorophenyl)-3′,3′-difluoro-[1,4′-bipiperidin]-4-yl)-4-((5-fluoro-4-(3-(2-oxopyridin-1(2H)-yl)phenyl)pyrimidin-2-yl)amino)cyclohexane-1-carboxamide (Compound 431). To a solution of 3-((4-(4-amino-3′,3′-difluoro-[1,4′-bipiperidin]-1′-yl)-3-fluorophenyl)amino)piperidine-2,6-dione (250 mg, 451 μmol, 1.00 eq, TFA), trans-4-((5-fluoro-4-(3-(2-oxopyridin-1(2H)-yl)phenyl)pyrimidin-2-yl)amino)cyclohexane-1-carboxylic acid (92.2 mg, 225 μmol, 0.500 eq) in DMF (5 mL) was added DIEA (175 mg, 1.36 mmol, 236 μL, 3.00 eq) and HATU (257 mg, 677 μmol, 1.50 eq) at 25 °C. The mixture was stirred at 25 °C for 12 h. LC-MS showed 3-((4-(4-amino-3′,3′-difluoro-[1,4′-bipiperidin]-1′-yl)-3-fluorophenyl)amino)piperidine-2,6-dione was consumed completely and the desired mass was detected. The reaction mixture was concentrated under reduced pressure. The residue was purified by prep-HPLC (column: Waters Xbridge BEH C18 100*30 mm*10 um; mobile phase: [H₂O (10 mM NH₄HCO₃)-ACN]; gradient: 40%-70% B over 8.0 min) to give the title compound (Compound 431) (123 mg, 142 μmol, 31.5% yield, 95.5% purity) as a white solid. LCMS (ES⁺): m/z 830.3 [M+H]⁺.

### Example 452. Synthesis of trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(2-oxopyridin-1(2H)-yl)phenyl)pyrimidin-2-yl)amino)cyclohexane-1-carboxamide (Compound 452)

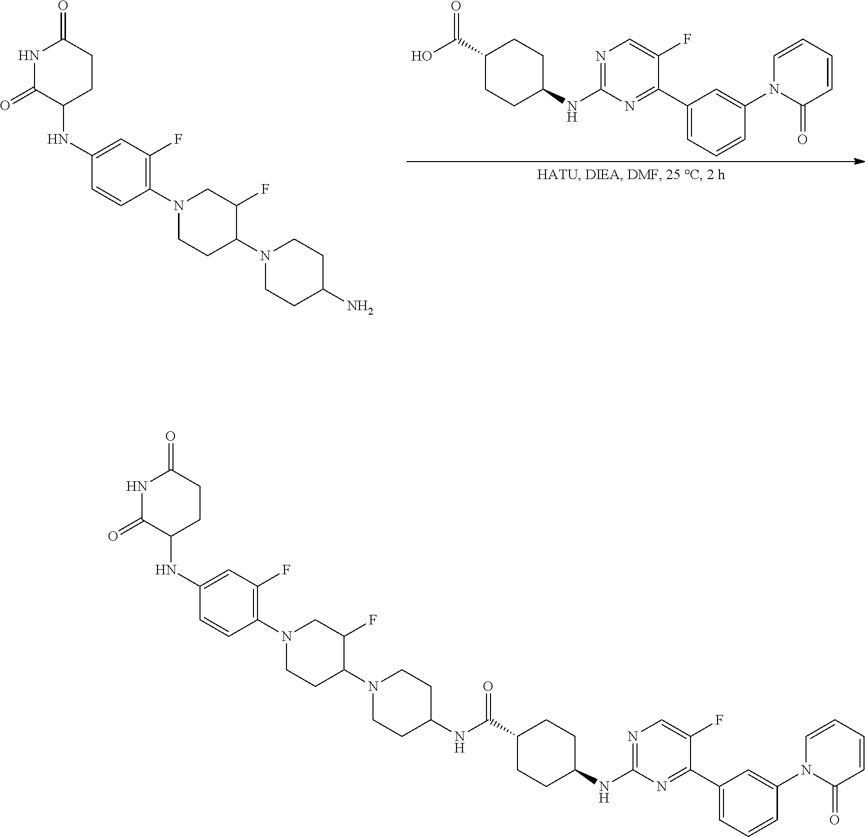 [SMILES: NC1CCN(C2CCN(c3ccc(NC4CCC(=O)NC4=O)cc3F)CC2F)CC1 ; O=c1ccccn1-c1cccc(-c2nc(N[C@H]3CC[C@H](C(=O)O)CC3)ncc2F)c1 ; O=C1CCC(Nc2ccc(N3CCC(N4CCC(NC(=O)[C@H]5CC[C@H](Nc6ncc(F)c(-c7cccc(-n8ccccc8=O)c7)n6)CC5)CC4)C(F)C3)c(F)c2)C(=O)N1]

Synthesis of trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(2-oxopyridin-1(2H)-yl)phenyl)pyrimidin-2-yl)amino)cyclohexane-1-carboxamide (Compound 452). To a solution of 3-((4-(4-amino-3′-fluoro-[1,4′-bipiperidin]-1′-yl)-3-fluorophenyl)amino)piperidine-2,6-dione (300 mg, 560 μmol, 1.00 eq, TFA) in DMF (2 mL) was added DIEA (217 mg, 1.68 mmol, 292 μL, 3.00 eq), trans-4-((5-fluoro-4-(3-(2-oxopyridin-1(2H)-yl)phenyl)pyrimidin-2-yl)amino)cyclohexane-1-carboxylic acid (228 mg, 560 μmol, 1.00 eq) and HATU (213 mg, 560 μmol, 1.00 eq) at 25 °C. The mixture was stirred at 25 °C for 2 h. LC-MS showed 3-((4-(4-amino-3′-fluoro-[1,4′-bipiperidin]-1′-yl)-3-fluorophenyl)amino)piperidine-2,6-dione was consumed completely and the desired mass was detected. The reaction mixture was concentrated under reduced pressure. The residue was purified by prep-HPLC (column: Phenomenex Luna C18 100*30 mm*3 um; mobile phase: [H₂O (0.2% FA)-ACN]; gradient: 5%-45% B over 8.0 min) to give the title compound (Compound 452) (45.0 mg, 51.7 μmol, 9.23% yield, 94.1% purity, 0.170 FA) was obtained as a pale yellow solid. LCMS (ES⁺): m/z 812.3 [M+H]⁺.

### Example 453. Synthesis of trans-3-(6-(1-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)-1-methyl-1H-indazol-3-yl)piperidine-2,6-dione (Compound 385)

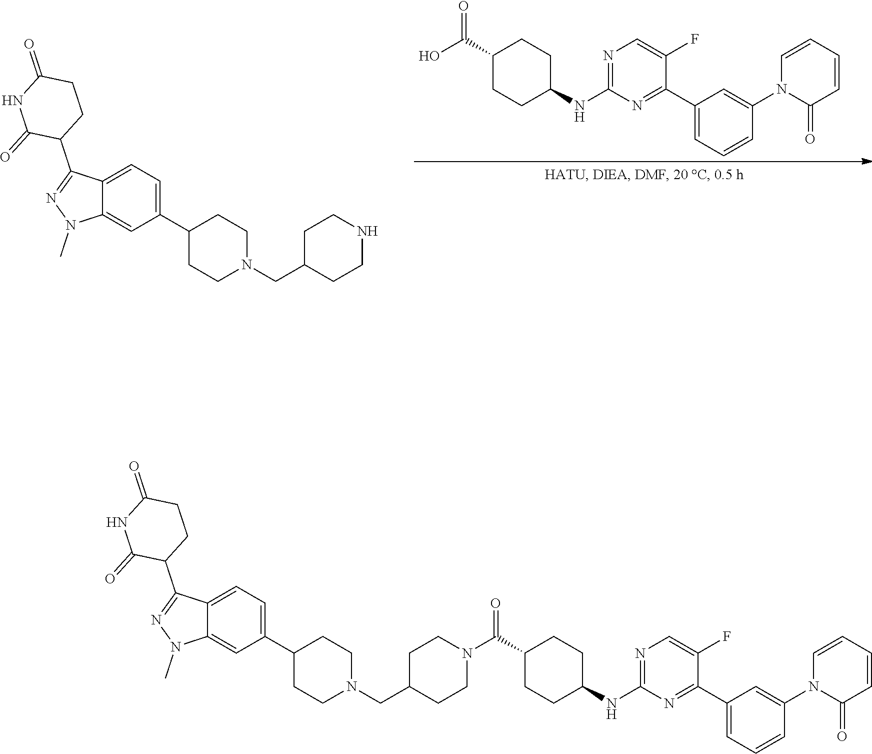 [SMILES: Cn1nc(C2CCC(=O)NC2=O)c2ccc(C3CCN(CC4CCNCC4)CC3)cc21 ; O=c1ccccn1-c1cccc(-c2nc(N[C@H]3CC[C@H](C(=O)O)CC3)ncc2F)c1 ; Cn1nc(C2CCC(=O)NC2=O)c2ccc(C3CCN(CC4CCN(C(=O)[C@H]5CC[C@H](Nc6ncc(F)c(-c7cccc(-n8ccccc8=O)c7)n6)CC5)CC4)CC3)cc21]

Synthesis of trans-3-(6-(1-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)-1-methyl-1H-indazol-3-yl)piperidine-2,6-dione (Compound 385). To a solution of trans-4-((5-fluoro-4-(3-(2-oxopyridin-1(2H)-yl)phenyl)pyrimidin-2-yl)amino)cyclohexane-1-carboxylic acid (387 mg, 949 μmol, 0.600 eq) in DMF (5 mL) were added HATU (661 mg, 1.74 mmol, 1.10 eq) and DIEA (1.02 g, 7.90 mmol, 1.38 μL, 5.00 eq) at 20 °C, then 3-(1-methyl-6-(1-(piperidin-4-ylmethyl)piperidin-4-yl)-1H-indazol-3-yl)piperidine-2,6-dione (850 mg, 1.58 mmol, 1.00 eq, TFA) was added to the mixture, the mixture was stirred at 20 °C for 0.5 h. LCMS showed 3-(1-methyl-6-(1-(piperidin-4-ylmethyl)piperidin-4-yl)-1H-indazol-3-yl)piperidine-2,6-dione was consumed and the desired target MS was detected. The mixture was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 20%-50% B over 8.0 min) to give the title compound (Compound 385) (85.3 mg, 102 μmol, 6.43% yield, 97.1% purity) as a yellow solid. LCMS (ES⁺): m/z 814.4 [M+H]⁺

### Example 454. Synthesis of trans-(R)-3-(6-(1-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)-1-methyl-1H-indazol-3-yl)piperidine-2,6-dione (Compound 422) and trans-(S)-3-(6-(1-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)-1-methyl-1H-indazol-3-yl)piperidine-2,6-dione (Compound 438) (The Two Isomers were Assigned Arbitrarily)

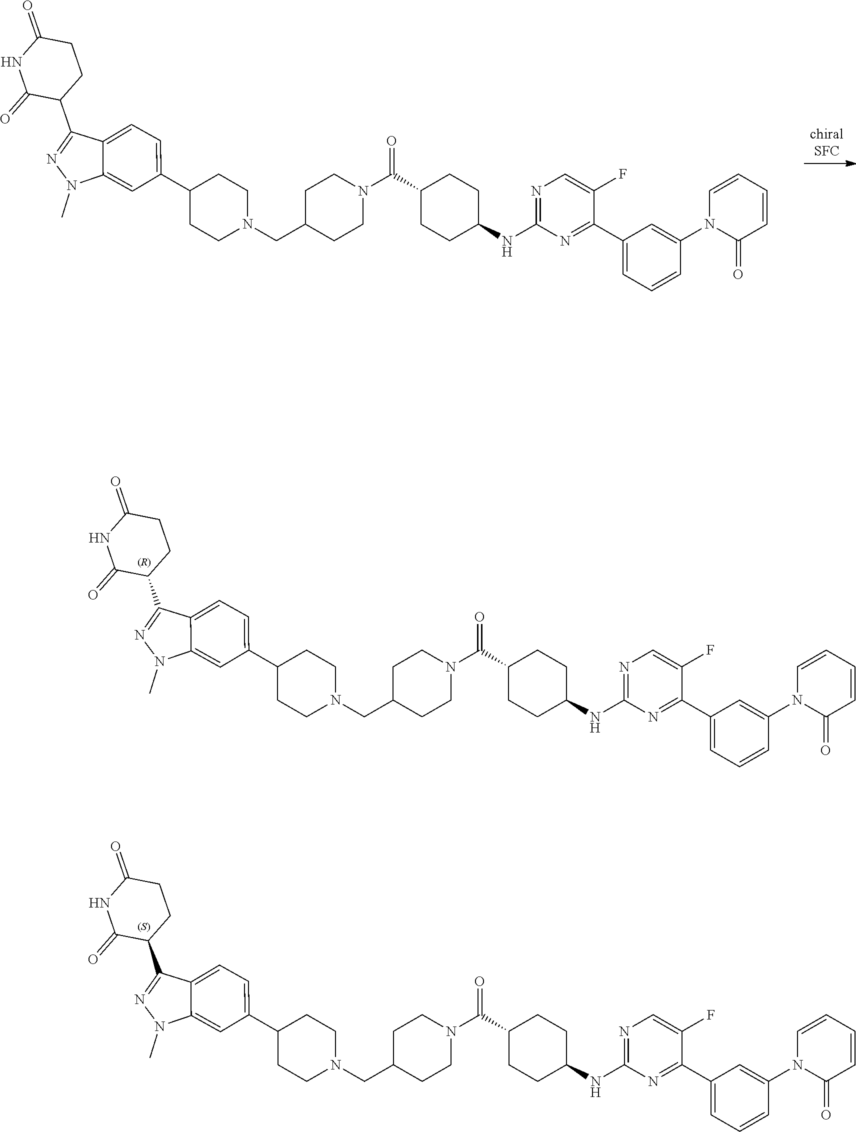

Synthesis of trans-(R)-3-(6-(1-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)-1-methyl-1H-indazol-3-yl)piperidine-2,6-dione (Compound 422) and trans-(S)-3-(6-(1-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)-1-methyl-1H-indazol-3-yl)piperidine-2,6-dione (Compound 438) (the two isomers were assigned arbitrarily). Trans-3-(6-(1-((1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)-1-methyl-1H-indazol-3-yl)piperidine-2,6-dione (Compound 385) (80.0 mg, 98.3 μmol, 1.00 eq) was separated by chiral SFC (column: REGIS(S,S)WHELK-O1 (250 mm*25 mm, 10 um); mobile phase: [CO₂—IPA:ACN=1:1]; B %: 70%, isocratic elution mode) to give the title compound (Compound 422) (21.5 mg, 25.8 mol, 26.3% yield, 97.8% purity, 97.6% ee) (retention time: 1.921, peak 1) as a white solid. LCMS (ES⁺): m/z 814.4 [M+H]⁺ and the title compound (Compound 438) (8.70 mg, 10.3 μmol, 10.4% yield, 96.0% purity, 94.3% ee) (retention time: 3.438, peak 2) as a white solid. LCMS (ES⁺): m/z 814.4 [M+H]⁺

### Example 455. Synthesis of trans-3-((3-fluoro-4-(1-((1-(-4-((5-fluoro-4-(3-(2-oxopyridin-1(2H)-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 409)

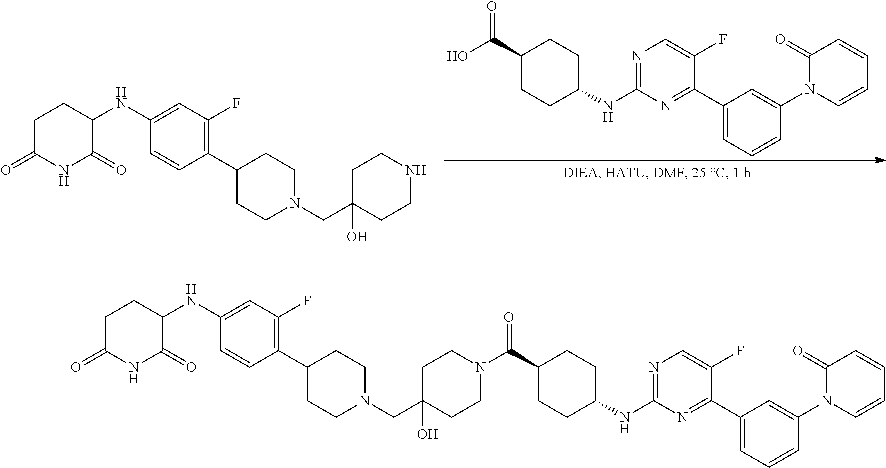 [SMILES: O=C1CCC(Nc2ccc(C3CCN(CC4(O)CCNCC4)CC3)c(F)c2)C(=O)N1 ; O=c1ccccn1-c1cccc(-c2nc(N[C@H]3CC[C@H](C(=O)O)CC3)ncc2F)c1 ; O=C1CCC(Nc2ccc(C3CCN(CC4(O)CCN(C(=O)[C@H]5CC[C@H](Nc6ncc(F)c(-c7cccc(-n8ccccc8=O)c7)n6)CC5)CC4)CC3)c(F)c2)C(=O)N1]

Synthesis of trans-3-((3-fluoro-4-(1-((1-(-4-((5-fluoro-4-(3-(2-oxopyridin-1(2H)-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 409). To a solution of trans-4-((5-fluoro-4-(3-(2-oxopyridin-1(2H)-yl)phenyl)pyrimidin-2-yl)amino)cyclohexane-1-carboxylic acid (383 mg, 939 μmol, 0.500 eq) in DMF (8 mL) were added HATU (250 mg, 657 μmol, 337 μL, 0.350 eq), DIEA (728 mg, 5.63 mmol, 981 μL, 3.00 eq) and 3-((3-fluoro-4-(1-((4-hydroxypiperidin-4-yl)methyl)piperidin-4-yl)phenyl)amino)piperidine-2,6-dione (1.00 g, 1.88 mmol, 1.00 eq, TFA) at 25 °C. The mixture was stirred at 25 °C for 1 h. LC-MS showed 3-((3-fluoro-4-(1-((4-hydroxypiperidin-4-yl)methyl)piperidin-4-yl)phenyl)amino)piperidine-2,6-dione was consumed completely, and the desired mass was detected. The reaction mixture was concentrated under reduced pressure to give a residue. The residue was purified by prep-HPLC (column: Waters Xbridge BEH C18 250*50 mm*10 um; mobile phase: [H₂O (10 mM NH₄HCO₃)-ACN]; gradient: 30%-60% B over 10.0 min) to give the title compound (Compound 409) (183 mg, 221 μmol, 11.7% yield, 97.4% purity) as a white solid. LCMS (ES⁺): m/z 809.4 [M+H]⁺

### Example 456. Synthesis of trans-(R)-3-((3-fluoro-4-(1-((1-(-4-((5-fluoro-4-(3-(2-oxopyridin-1(2H)-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 442) and trans-(S)-3-((3-fluoro-4-(1-((1-(-4-((5-fluoro-4-(3-(2-oxopyridin-1(2H)-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 443) (The Two Isomers were Assigned Arbitrarily)

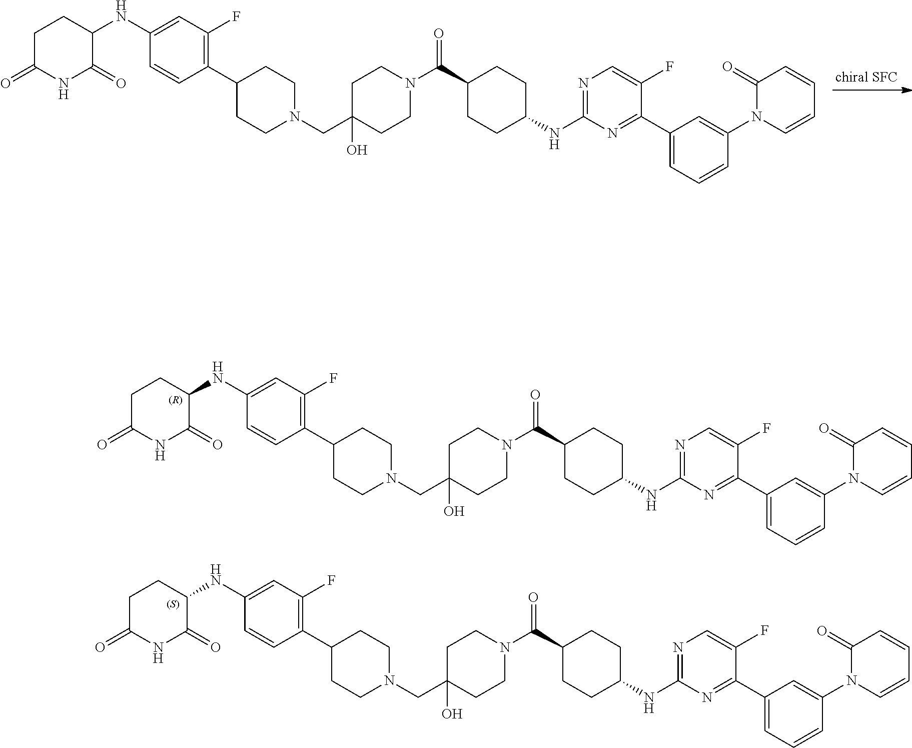

Synthesis of trans-(R)-3-((3-fluoro-4-(1-((1-(-4-((5-fluoro-4-(3-(2-oxopyridin-1(2H)-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 442) and trans-(S)-3-((3-fluoro-4-(1-((1-(-4-((5-fluoro-4-(3-(2-oxopyridin-1(2H)-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 443) (the two isomers were assigned arbitrarily). Trans-3-((3-fluoro-4-(1-((1-(-4-((5-fluoro-4-(3-(2-oxopyridin-1(2H)-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 409) (170 mg, 210 μmol, 1.00 eq) was separated by chiral SFC (column: ChiralPak IH, 250*30 mm, 10 um; mobile phase: [CO₂-IPA (0.1% IPAm)]; B %: 60%, isocratic elution mode) to give the title compound (Compound 442) (52.3 mg, 64.7 μmol, 30.8% yield, 100% purity, 100% ee) (retention time: 1.93, peak 1) as a white solid. LCMS (ES⁺): m/z 809.4 [M+H]⁺ and the title compound (Compound 443) (50.7 mg, 60.8 μmol, 28.9% yield, 97.04% purity, 98.8% ee) (retention time: 4.17, peak 2) as a white solid. LCMS (ES⁺): m/z 809.5 [M+H]⁺

### Example 457. Synthesis of trans-3-((3-fluoro-4-(4-((1-(4-((5-fluoro-4-(3-(2-oxopyridin-1(2H)-yl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 392)

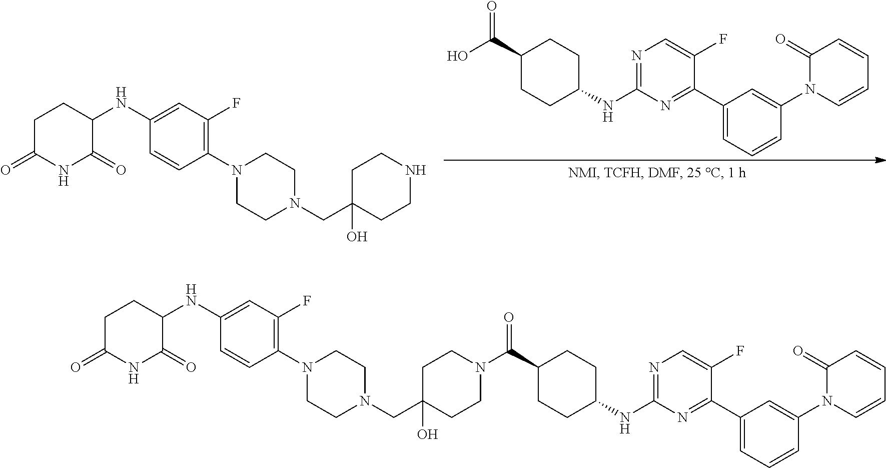 [SMILES: O=C1CCC(Nc2ccc(N3CCN(CC4(O)CCNCC4)CC3)c(F)c2)C(=O)N1 ; O=c1ccccn1-c1cccc(-c2nc(N[C@H]3CC[C@H](C(=O)O)CC3)ncc2F)c1 ; O=C1CCC(Nc2ccc(N3CCN(CC4(O)CCN(C(=O)[C@H]5CC[C@H](Nc6ncc(F)c(-c7cccc(-n8ccccc8=O)c7)n6)CC5)CC4)CC3)c(F)c2)C(=O)N1]

Synthesis of trans-3-((3-fluoro-4-(4-((1-(4-((5-fluoro-4-(3-(2-oxopyridin-1(2H)-yl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 392). To a solution of trans-4-[[5-fluoro-4-[3-(2-oxo-1-pyridyl)phenyl]pyrimidin-2-yl]amino]cyclohexanecarboxylic acid (260 mg, 637 μmol, 1.00 eq) in DMF (10 mL) were added NMI (310.5 mg, 3.78 mmol, 302 μL, 6.00 eq) and TCFH (396 mg, 1.41 mmol, 2.20 eq) at 25 °C. Then 3-((3-fluoro-4-(4-((4-hydroxypiperidin-4-yl)methyl)piperazin-1-yl)phenyl)amino)piperidine-2,6-dione (340 mg, 637 μmol, 1.00 eq, TFA) was added to the reaction mixture at 25 °C. The mixture was stirred at 25 °C for 1 h. LCMS showed 3-((3-fluoro-4-(4-((4-hydroxypiperidin-4-yl)methyl)piperazin-1-yl)phenyl)amino)piperidine-2,6-dione was consumed completely and the desired mass was detected. The reaction was concentrated under reduced to give a residue. The residue was purified by prep-HPLC (column: Phenomenex Gemini-NX 80*40 mm*3 um; mobile phase: [H₂O (10 mM NH₄HCO₃)-ACN]; gradient: 25%-50% B over 8.0 min) to give the title compound (Compound 392) (67.1 mg, 78.5 μmol, 12.3% yield, 94.7% purity) as a white solid. LCMS (ES⁺): m/z 810.4 [M+H]⁺

### Example 458. Synthesis of trans-(R)-3-((3-fluoro-4-(4-((1-(4-((5-fluoro-4-(3-(2-oxopyridin-1(2H)-yl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 423) and trans-(S)-3-((3-fluoro-4-(4-((1-(4-((5-fluoro-4-(3-(2-oxopyridin-1(2H)-yl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 439) (The Two Isomers were Assigned Arbitrarily)

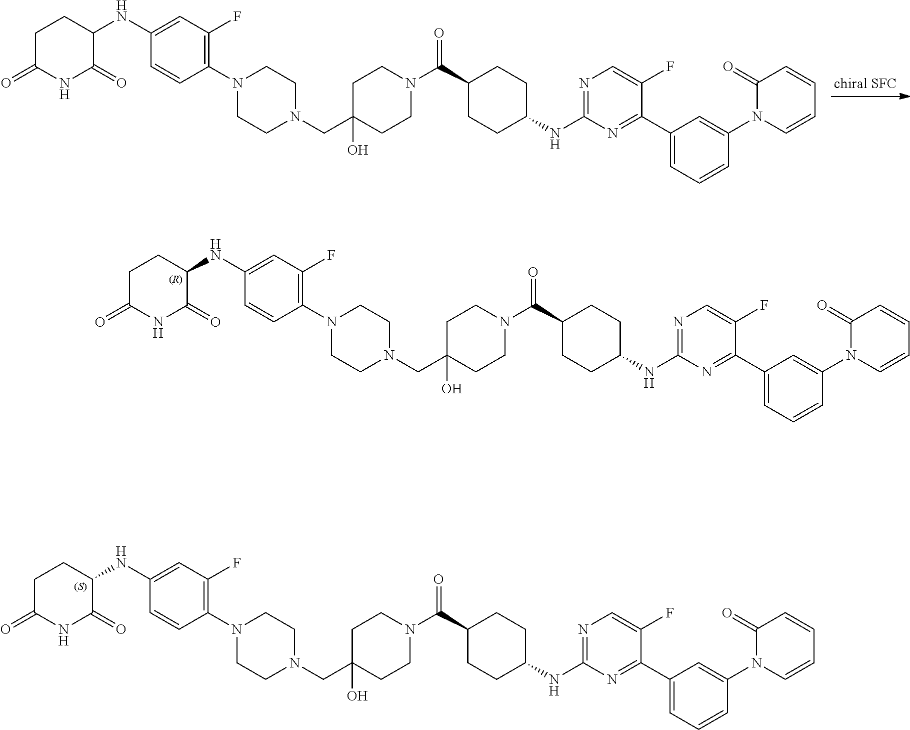

Synthesis of trans-(R)-3-((3-fluoro-4-(4-((1-(4-((5-fluoro-4-(3-(2-oxopyridin-1(2H)-yl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 423) and trans-(S)-3-((3-fluoro-4-(4-((1-(4-((5-fluoro-4-(3-(2-oxopyridin-1(2H)-yl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 439) (the two isomers were assigned arbitrarily). trans-3-((3-fluoro-4-(4-((1-(4-((5-fluoro-4-(3-(2-oxopyridin-1(2H)-yl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 392) was separated by SFC (column: REGIS (s, s) WHELK-O1 (250 mm*30 mm, 5 um); mobile phase: [CO₂-IPA:CAN=1:1]; B %: 65%, isocratic elution mode) to give the title compound (Compound 423) (14.0 mg, 16.2 μmol, 21.8% yield, 93.6% purity, 98.0% ee) (retention time: 1.82, peak 1) as a white solid. LCMS (ES⁺): m/z 810.4 [M+H]⁺ and the title compound (Compound 439) (19.0 mg, 21.5 μmol, 29.1% yield, 91.8% purity, 94.4% ee) (retention time: 2.10, peak 2) as a white solid. LCMS (ES⁺): m/z 810.4 [M+H]⁺

### Example 459. Synthesis of trans-3-((3-fluoro-4-(4-((4-(4-((5-fluoro-4-(3-(2-oxopyridin-1(2H)-yl)phenyl)pyrimidin-2-yl)amino)cyclohexane-1-carbonyl)piperazin-1-yl)methyl)-4-hydroxypiperidin-1-yl)phenyl)amino)piperidine-2,6-dione (Compound 410)

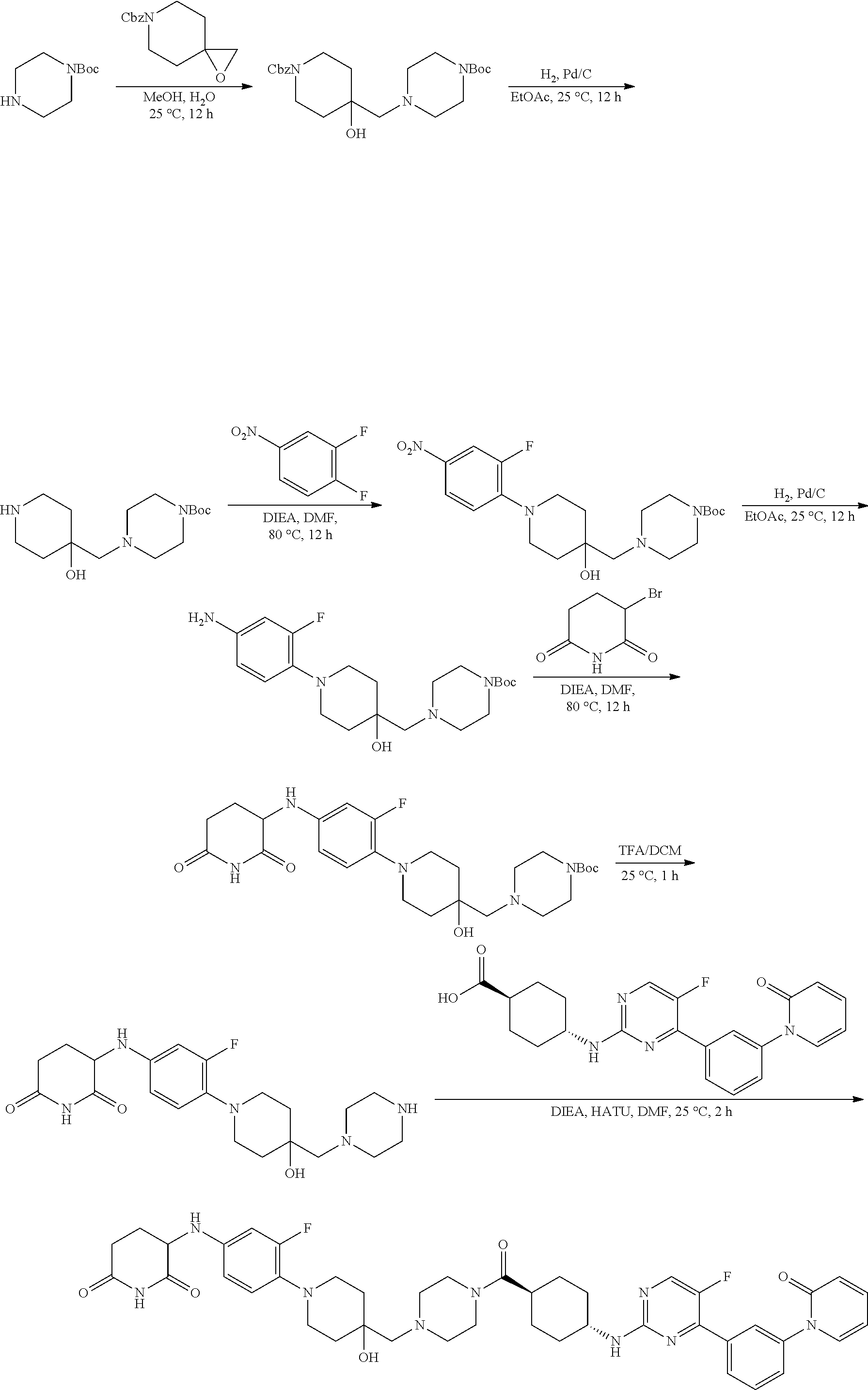

Synthesis of tert-butyl 4-((1-((benzyloxy)carbonyl)-4-hydroxypiperidin-4-yl)methyl)piperazine-1-carboxylate. To a solution of benzyl 1-oxa-6-azaspiro[2.5]octane-6-carboxylate (9.30 g, 37.6 mmol, 1.00 eq) in MeOH (90 mL) and H₂O (90 mL) was added tert-butyl piperazine-1-carboxylate (14.0 g, 75.2 mmol, 2.00 eq) at 25 °C. The mixture was stirred at 25 °C for 12 h. LCMS showed the desired mass was detected. The reaction mixture was concentrated under reduced pressure to remove MeOH. The solid was precipitated, it was filtered and the filter cake was dried under reduced pressure to give the title compound (20.0 g, crude) as a white solid. LCMS (ES⁺): m/z 434.2 [M+H]⁺.

Synthesis of tert-butyl 4-((4-hydroxypiperidin-4-yl)methyl)piperazine-1-carboxylate. 10% Pd/C (14.0 g, 50% purity) was added to EtOAc (140 mL) at 25 °C under N₂ atmosphere, then tert-butyl 4-((1-((benzyloxy)carbonyl)-4-hydroxypiperidin-4-yl)methyl)piperazine-1-carboxylate (5.00 g, 11.5 mmol, 1.00 eq) was added at 25 °C, the suspension was degassed and purged with H₂ for three times. The mixture was stirred at 25 °C for 12 h under H₂ (15 Psi) atmosphere. LCMS showed tert-butyl 4-((1-((benzyloxy)carbonyl)-4-hydroxypiperidin-4-yl)methyl)piperazine-1-carboxylate was consumed completely and the desired mass was detected. The reaction mixture was filtered and the filtrate was concentrated under reduced pressure to give the title compound (6.10 g, crude) as a brown oil. LCMS (ES⁺): m/z 300.3 [M+H]⁺.

Synthesis of tert-butyl 4-((1-(2-fluoro-4-nitrophenyl)-4-hydroxypiperidin-4-yl)methyl)piperazine-1-carboxylate. To a solution of tert-butyl 4-((4-hydroxypiperidin-4-yl)methyl)piperazine-1-carboxylate (6.00 g, 20.0 mmol, 1.00 eq) in DMF (50 mL) were added DIEA (7.77 g, 60.1 mmol, 10.5 mL, 3.00 eq) and 1,2-difluoro-4-nitro-benzene (3.19 g, 20.0 mmol, 2.22 mL, 1.00 eq) at 25 °C. The mixture was stirred at 80 °C for 12 h. LCMS showed tert-butyl 4-((4-hydroxypiperidin-4-yl)methyl)piperazine-1-carboxylate was consumed completely and the desired mass was detected. Water (50 mL) was added to the mixture at 25 °C. The reaction was filtered and the filter cake was dried under reduced pressure to give the title compound (7.10 g, crude) as a yellow solid. LCMS (ES⁺): m/z 439.3 [M+H]⁺.

Synthesis of tert-butyl 4-((1-(4-amino-2-fluorophenyl)-4-hydroxypiperidin-4-yl)methyl)piperazine-1-carboxylate. 10% Pd/C (4.00 g, 50% purity) was added to EtOAc (80 mL) at 25 °C under N₂ atmosphere, then tert-butyl 4-((1-(2-fluoro-4-nitrophenyl)-4-hydroxypiperidin-4-yl)methyl)piperazine-1-carboxylate (4.00 g, 9.12 mmol, 1.00 eq) was added at 25 °C, the suspension was degassed and purged with H₂ for three times. The mixture was stirred at 25 °C for 12 h under H₂ (15 Psi) atmosphere. LCMS showed tert-butyl 4-((1-(2-fluoro-4-nitrophenyl)-4-hydroxypiperidin-4-yl)methyl)piperazine-1-carboxylate was consumed completely and the desired mass was detected. The mixture was filtered and the filtrate was concentrated under reduced pressure to give the title compound (5.20 g, crude) as a yellow solid. LCMS (ES⁺): m/z 409.3 [M+H]⁺.

Synthesis of tert-butyl 4-((1-(4-((2,6-dioxopiperidin-3-yl)amino)-2-fluorophenyl)-4-hydroxypiperidin-4-yl)methyl)piperazine-1-carboxylate. To a solution of tert-butyl 4-((1-(4-amino-2-fluorophenyl)-4-hydroxypiperidin-4-yl)methyl)piperazine-1-carboxylate (3.00 g, 7.34 mmol, 1.00 eq) in DMF (50 mL) were added DIEA (2.85 g, 22.0 mmol, 3.84 mL, 3.00 eq) and 3-bromopiperidine-2,6-dione (3.53 g, 18.4 mmol, 2.50 eq) at 25 °C. The mixture was stirred at 80 °C for 12 h. LCMS showed tert-butyl 4-((1-(4-amino-2-fluorophenyl)-4-hydroxypiperidin-4-yl)methyl)piperazine-1-carboxylate was consumed completely and the desired mass was detected. Water (50 mL) was added to the mixture at 25 °C. The mixture was filtered and the filter cake was dried under reduced pressure to give the title compound (2.50 g, crude) as a black solid. LCMS (ES⁺): m/z 520.3 [M+H]⁺

Synthesis of 3-((3-fluoro-4-(4-hydroxy-4-(piperazin-1-ylmethyl)piperidin-1-yl)phenyl)amino)piperidine-2,6-dione. To a solution of tert-butyl 4-((1-(4-((2,6-dioxopiperidin-3-yl)amino)-2-fluorophenyl)-4-hydroxypiperidin-4-yl)methyl)piperazine-1-carboxylate (1.00 g, 1.92 mmol, 1.00 eq) in DCM (9 mL) was added TFA (3 mL) at 25 °C. The mixture was stirred at 25 °C for 1 h. LCMS showed tert-butyl 4-((1-(4-((2,6-dioxopiperidin-3-yl)amino)-2-fluorophenyl)-4-hydroxypiperidin-4-yl)methyl)piperazine-1-carboxylate was consumed completely and the desired mass was detected. The reaction mixture was concentrated under reduced pressure to give the title compound (1.00 g, crude, TFA) as a yellow oil. LCMS (ES⁺): m/z 420.2 [M+H]⁺.

Synthesis of trans-3-((3-fluoro-4-(4-((4-(4-((5-fluoro-4-(3-(2-oxopyridin-1(2H)-yl)phenyl)pyrimidin-2-yl)amino)cyclohexane-1-carbonyl)piperazin-1-yl)methyl)-4-hydroxypiperidin-1-yl)phenyl)amino)piperidine-2,6-dione (Compound 410). To a solution of trans-4-[[5-fluoro-4-[3-(2-oxo-1-pyridyl)phenyl]pyrimidin-2-yl]amino]cyclohexanecarboxylic acid (230 mg, 562 μmol, 0.300 eq) in DMF (6 mL) were added HATU (249 mg, 656 μmol, 0.35 eq), DIEA (1.21 g, 9.37 mmol, 1.63 mL, 5.00 eq) and 3-((3-fluoro-4-(4-hydroxy-4-(piperazin-1-ylmethyl)piperidin-1-yl)phenyl)amino)piperidine-2,6-dione (1.00 g, 1.87 mmol, 1.00 eq, TFA) at 25 °C. The mixture was stirred at 25 °C for 2 h. LCMS showed 3-((3-fluoro-4-(4-hydroxy-4-(piperazin-1-ylmethyl)piperidin-1-yl)phenyl)amino)piperidine-2,6-dione was consumed completely and the desired mass was detected. The mixture was filtered and the filtrate was concentrated under reduced pressure to give a residue. The residue was purified by prep-HPLC (column: Phenomenex luna C18 100*40 mm*5 um; mobile phase: [H₂O (0.2% FA)-ACN]; gradient: 10%-45% B over 8.0 min) to give the title compound (Compound 410) (132 mg, 151 μmol, 8.04% yield, 97.8% purity, FA) as a gray solid. LCMS (ES⁺): m/z 810.4 [M+H]⁺

### Example 460. Synthesis of trans-1-((1-(4-((2,6-dioxopiperidin-3-yl)amino)-2-fluorophenyl)piperidin-4-yl)methyl)-N-((1r,4r)-4-((5-fluoro-4-(3-(2-oxopyridin-1(2H)-yl)phenyl)pyrimidin-2-yl)amino)cyclohexyl)piperidine-4-carboxamide (Compound 540

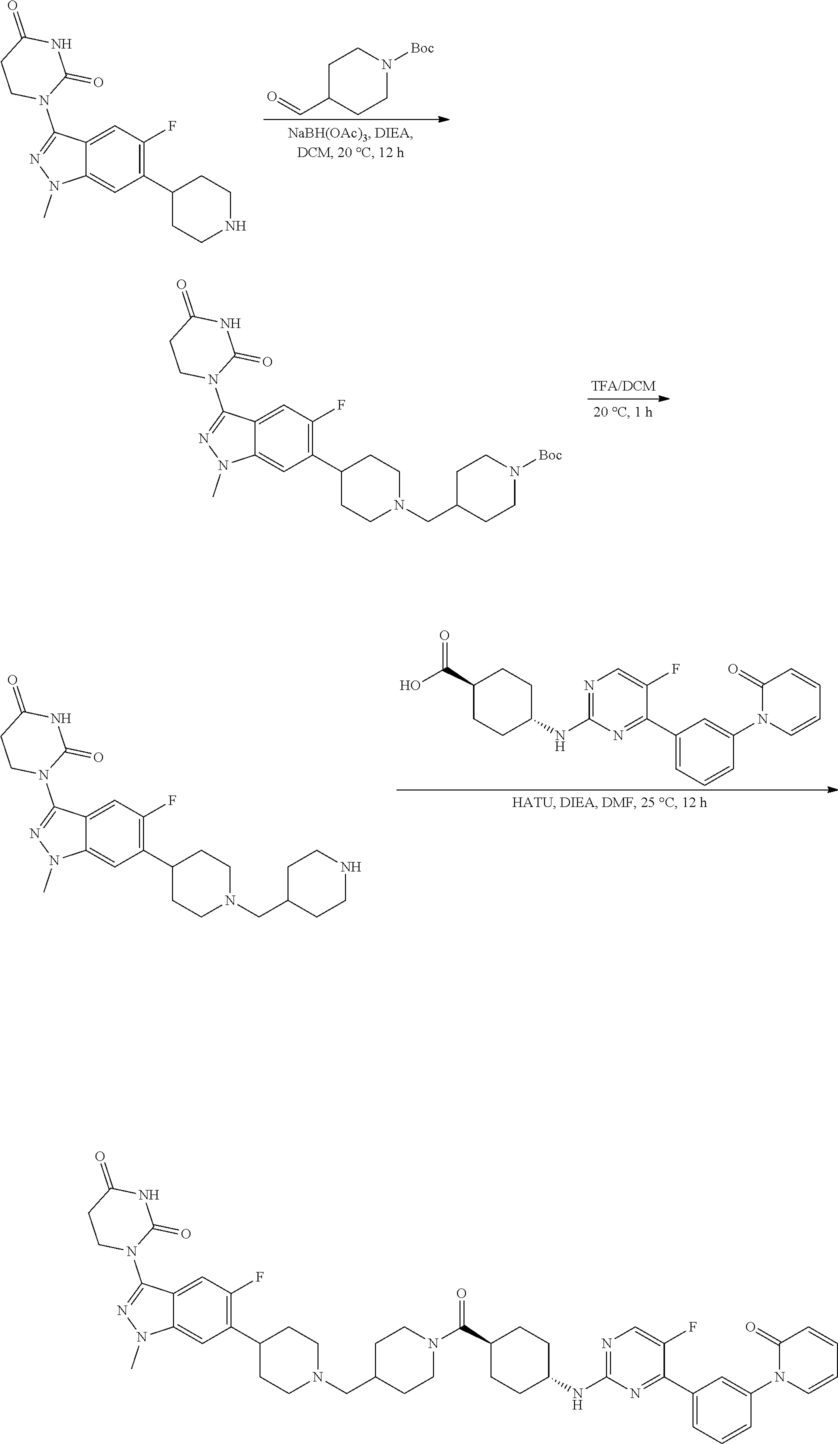

Synthesis of tert-butyl 4-((4-(3-(2,4-dioxotetrahydropyrimidin-1(2H)-yl)-5-fluoro-1-methyl-1H-indazol-6-yl)piperidin-1-yl)methyl)piperidine-1-carboxylate. To a solution of 1-(5-fluoro-1-methyl-6-(piperidin-4-yl)-1H-indazol-3-yl)dihydropyrimidine-2,4(1H,3H)-dione (2.00 g, 5.79 mmol, 1.00 eq) and tert-butyl 4-formylpiperidine-1-carboxylate (1.24 g, 5.79 mmol, 1.00 eq) in DCM (30 mL) were added DIEA (748 mg, 5.79 mmol, 1.01 mL, 1.00 eq) and NaBH(OAc)₃ (1.23 g, 5.79 mmol, 1.00 eq) at 20 °C. The mixture was stirred at 20 °C for 12 h. LC-MS showed that 1-(5-fluoro-1-methyl-6-(piperidin-4-yl)-1H-indazol-3-yl)dihydropyrimidine-2,4(1H,3H)-dione was consumed completely and desired mass was detected. The residue was diluted with H₂O (30 mL) and extracted with DCM (30 mL*3). The combined organic layers were washed with brine (30 mL), dried over anhydrous Na₂SO₄, filtered and concentrated under reduced pressure. The residue was purified by column chromatography (SiO₂, Petroleum ether/Ethyl acetate=1/0 to 0/1) to give the title compound (3.0 g, crude) as a brown oil. LCMS (ES⁺): m/z 543.3 [M+H]⁺.

Synthesis of 1-(5-fluoro-1-methyl-6-(1-(piperidin-4-ylmethyl)piperidin-4-yl)-1H-indazol-3-yl)dihydropyrimidine-2,4(1H,3H)-dione. To a solution of tert-butyl 4-((4-(3-(2,4-dioxotetrahydropyrimidin-1(2H)-yl)-5-fluoro-1-methyl-1H-indazol-6-yl)piperidin-1-yl)methyl)piperidine-1-carboxylate (600 mg, 1.11 mmol, 1.00 eq) in DCM (6 mL) was added TFA (3.07 g, 26.9 mmol, 2.00 mL, 24.4 eq) at 20 °C. The mixture was stirred at 20 °C for 1 h. LC-MS showed that tert-butyl 4-((4-(3-(2,4-dioxotetrahydropyrimidin-1(2H)-yl)-5-fluoro-1-methyl-1H-indazol-6-yl)piperidin-1-yl)methyl)piperidine-1-carboxylate was consumed completely and desired mass was detected. The reaction mixture was concentrated under reduced pressure to remove solvent to give the title compound (600 mg, crude, TFA) as a yellow oil. LCMS (ES⁺): m/z 443.2 [M+H]⁺

Synthesis of trans-1-(5-fluoro-6-(1-((1-((1r,4r)-4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)-1-methyl-1H-indazol-3-yl)dihydropyrimidine-2,4(1H,3H)-dione (Compound 540). To a solution of 1-(5-fluoro-1-methyl-6-(1-(piperidin-4-ylmethyl)piperidin-4-yl)-1H-indazol-3-yl)dihydropyrimidine-2,4(1H,3H)-dione (585 mg, 1.05 mmol, 1.59 eq, TFA) and trans-(1r,4r)-4-((5-fluoro-4-(3-(2-oxopyridin-1(2H)-yl)phenyl)pyrimidin-2-yl)amino)cyclohexane-1-carboxylic acid (270 mg, 661 μmol, 1.00 eq) in DMF (10 mL) were added DIEA (256 mg, 1.98 mmol, 345 μL, 3.00 eq) and HATU (628 mg, 1.65 mmol, 2.50 eq) at 20 °C. The mixture was stirred at 20 °C for 12 h. LC-MS showed that 1-(5-fluoro-1-methyl-6-(1-(piperidin-4-ylmethyl)piperidin-4-yl)-1H-indazol-3-yl)dihydropyrimidine-2,4(1H,3H)-dione was consumed completely and desired mass was detected. The residue was diluted with H₂O (10 mL) and extracted with EtOAc (10 mL*4). The combined organic layers were washed with brine (10 mL), dried over anhydrous Na₂SO₄, filtered and concentrated under reduced pressure to give a residue. The residue was purified by prep-HPLC (column: Phenomenex Luna C18 100*30 mm*3 um; mobile phase: [H₂O (0.2% FA)-ACN]; gradient: 10%-40% B over 8.0 min) to give the title compound (Compound 540) (156 mg, 177 μmol, 26.8% yield, 95.7% purity, 0.2 FA) as a white solid. LCMS (ES⁺): m/z 833.3 [M+H]⁺

### Example 461. Synthesis of trans-(1r,4R)—N-((1r,4R)-4-(4-(4-((2,6-dioxopiperidin-3-yl)amino)-2-fluorophenyl)piperazin-1-yl)cyclohexyl)-4-((5-fluoro-4-(3-(2-oxopyridin-1(2H)-yl)phenyl)pyrimidin-2-yl)amino)-N-methylcyclohexane-1-carboxamide (Compound 488)

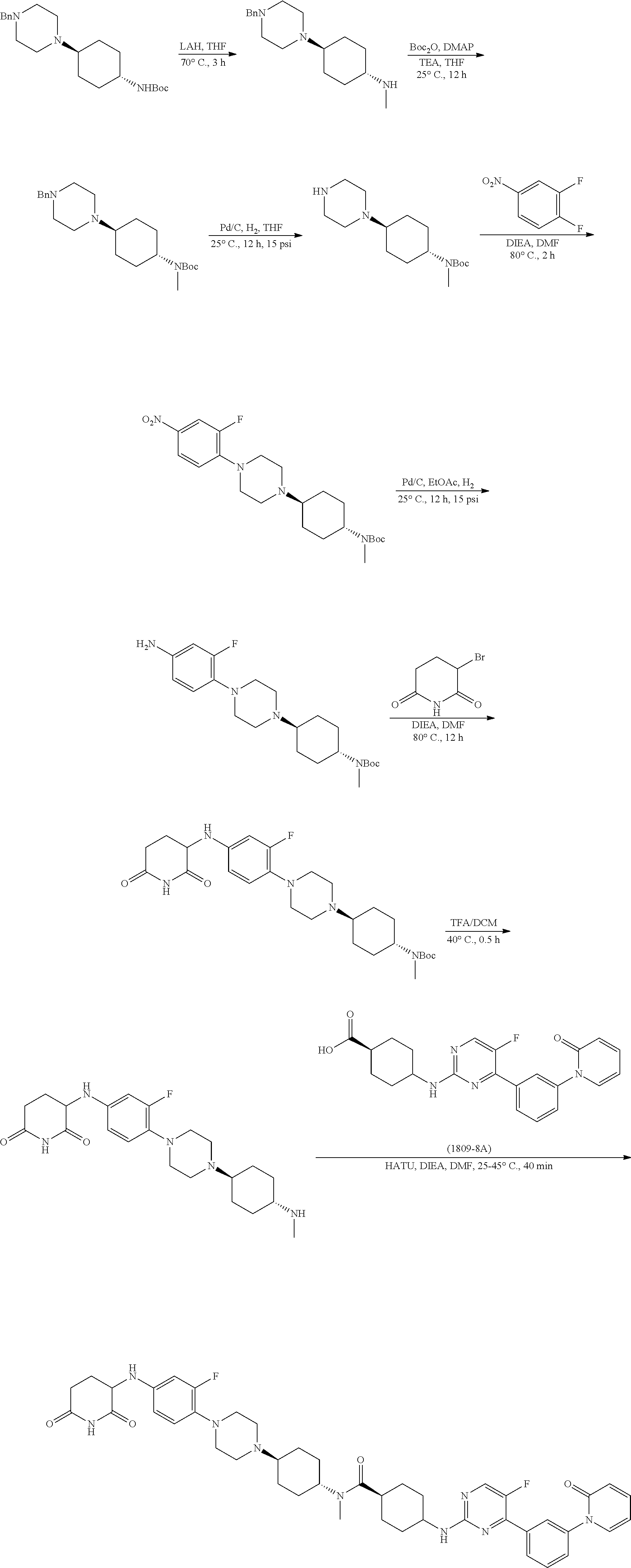

Synthesis of trans-4-(4-benzylpiperazin-1-yl)-N-methyl-cyclohexanamine. To a solution of trans-tert-butyl N-[4-(4-benzylpiperazin-1-yl)cyclohexyl]carbamate (3.50 g, 9.37 mmol, 1.00 eq) in THE (40 mL) was added LAH (2.50 M in THF, 9.37 mL, 2.50 eq) at 0 °C, the mixture was stirred at 70 °C for 3 h under N₂. LCMS showed that the desired mass was detected. The reaction was cooled to 25 °C, then it was quenched with H₂O (1 ml), 15% NaOH solution (1 ml) and H₂O (3 ml) under N₂, the mixture was stirred at 25 °C for 15 min. Then Na₂SO₄ (1 g) was added at 25 °C, the mixture was stirred at 25 °C for 15 min. Filtered and the filter cake was washed with EtOAc (90 ml), then the organic layer was dried over Na₂SO₄, filtered and the filtrate was concentrated under reduced pressure to give the title compound (2.70 g, crude) as a colorless oil liquid. LCMS (ES⁺): m/z 288.1 [M+H]⁺.

Synthesis of trans-tert-butyl N-[4-(4-benzylpiperazin-1-yl)cyclohexyl]-N-methyl-carbamate. To a solution of trans-4-(4-benzylpiperazin-1-yl)-N-methyl-cyclohexanamine (2.70 g, 9.39 mmol, 1.00 eq) and Boc₂O (6.15 g, 28.2 mmol, 6.47 mL, 3.00 eq) in THF (20 mL) was added TEA (1.90 g, 18.8 mmol, 2.61 mL, 2.00 eq) and DMAP (574 mg, 4.70 mmol, 0.500 eq) at 25 °C, the mixture was stirred at 25 °C for 12 h. LCMS showed that the trans-4-(4-benzylpiperazin-1-yl)-N-methyl-cyclohexanamine was consumed and the desired mass was detected. The mixture was concentrated under reduced pressure to give a residue. The residue was purified by MPLC (SiO₂, DCM/MeOH=1/0 to 20/1) to give the title compound (3.60 g, 9.29 mmol, 98.9% yield) as a brown solid. LCMS (ES⁺): m/z 388.3 [M+H]⁺.

Synthesis of trans-tert-butyl N-methyl-N-(4-piperazin-1-ylcyclohexyl)carbamate. To 10% Pd/C (4.00 g, 50% purity) was added THE (40 mL) at 25 °C under N₂. Then trans-tert-butyl N-[4-(4-benzylpiperazin-1-yl)cyclohexyl]-N-methyl-carbamate (3.50 g, 9.03 mmol, 1.00 eq) was added at 25 °C, the mixture was purged and degassed with H₂ for 3 times, and the resulting mixture was stirred at 25 °C for 12 h under H₂ (15 psi). LCMS showed that the trans-N-[4-(4-benzylpiperazin-1-yl)cyclohexyl]-N-methyl-carbamate was consumed and the desired mass was detected. The mixture was filtered and the filtrate was concentrated under reduced pressure to give the title compound (2.10 g, crude) as a brown solid. LCMS (ES⁺): m/z 298.3 [M+H]⁺.

Synthesis of trans-tert-butyl N-methyl-N-(4-piperazin-1-ylcyclohexyl)carbamate. To a solution of trans-tert-butyl N-methyl-N-(4-piperazin-1-ylcyclohexyl)carbamate (2.00 g, 6.72 mmol, 1.00 eq) and 1,2-difluoro-4-nitro-benzene (1.07 g, 6.72 mmol, 744 μL, 1.00 eq) in DMF (20 mL) was added DIEA (1.30 g, 10.09 mmol, 1.76 mL, 1.5 eq) at 25 °C, the mixture was stirred at 80 °C for 2 h. LCMS showed that the trans-tert-butyl N-methyl-N-(4-piperazin-1-ylcyclohexyl)carbamate was consumed and the desired mass was detected. The mixture was added to ice H₂O (150 mL) and filtered. The filter cake was dried under reduced pressure to give the title compound (2.10 g, crude) as a brown solid. LCMS (ES⁺): m/z 437.2 [M+H]⁺.

Synthesis of trans-tert-butyl N-[4-[4-(4-amino-2-fluoro-phenyl)piperazin-1-yl]cyclohexyl]-N-methyl-carbamate. To 10% Pd/C (3.00 g, 50% purity) was added EtOAc (30 mL) at 25 °C under N₂. Then trans-N-[4-[4-(2-fluoro-4-nitro-phenyl)piperazin-1-yl]cyclohexyl]-N-methyl-carbamate (2.40 g, 5.50 mmol, 1.00 eq) was added at 25 °C under N₂, the mixture was purged and degassed with H₂ for 3 times and the mixture was stirred at 25 °C for 12 h under H₂ (15 psi). LCMS showed that the trans-N-[4-[4-(2-fluoro-4-nitro-phenyl)piperazin-1-yl]cyclohexyl]-N-methyl-carbamate was consumed completely and the desired mass was detected. The mixture was filtered and the filtrate was concentrated under reduced pressure to give the title compound (2.20 g, crude) as a brown solid. LCMS (ES⁺): m/z 407.2 [M+H]⁺.

Synthesis of trans-tert-butyl N-[4-[4-[4-[(2,6-dioxo-3-piperidyl)amino]-2-fluoro-phenyl]piperazin-1-yl]cyclohexyl]-N-methyl-carbamate. To a solution of trans-tert-butyl N-[4-[4-(4-amino-2-fluoro-phenyl)piperazin-1-yl]cyclohexyl]-N-methyl-carbamate (2.20 g, 5.41 mmol, 1.00 eq) in DMF (40 mL) were added 3-bromopiperidine-2,6-dione (3.12 g, 16.2 mmol, 3.00 eq) and DIEA (2.10 g, 16.2 mmol, 2.83 mL, 3.00 eq) at 25 °C, the mixture was stirred at 80 °C for 12 h. LCMS showed that the trans-tert-butyl N-[4-[4-(4-amino-2-fluoro-phenyl)piperazin-1-yl]cyclohexyl]-N-methyl-carbamate was consumed completely and the desired mass was detected. To the mixture was added H₂O (40 mL) and the mixture was extracted with EtOAc (40 mL*3). The combined organic layers were washed with brine (50 mL*3), dried over Na₂SO₄, filtered and the filtrate was concentrated under reduced pressure to give a residue. The residue was purified by MPLC (SiO₂, DCM/MeOH=1/0 to 20/1) to give the title compound (1.00 g, 1.93 mmol, 35.7% yield) as a black solid. LCMS (ES⁺): m/z 518.4 [M+H]⁺.

Synthesis of trans-3-[3-fluoro-4-[4-[4-(methylamino)cyclohexyl]piperazin-1-yl]anilino]piperidine-2,6-dione. To a solution of trans-tert-butyl N-[4-[4-[4-[(2,6-dioxo-3-piperidyl)amino]-2-fluoro-phenyl]piperazin-1-yl]cyclohexyl]-N-methyl-carbamate (150 mg, 290 μmol, 1.00 eq) in DCM (3 mL) was added TFA (1 mL) at 25 °C, the mixture was stirred at 40 °C for 0.5 h. LCMS showed that the trans-tert-butyl N-[4-[4-[4-[(2,6-dioxo-3-piperidyl)amino]-2-fluoro-phenyl]piperazin-1-yl]cyclohexyl]-N-methyl-carbamate was consumed completely and the desired mass was detected. The mixture was concentrated under reduced pressure to give the title compound (155 mg, crude, TFA) as a black oil liquid. LCMS (ES⁺): m/z 418.2 [M+H]⁺.

Synthesis of trans-(1r,4R)—N-((1r,4R)-4-(4-(4-((2,6-dioxopiperidin-3-yl)amino)-2-fluorophenyl)piperazin-1-yl)cyclohexyl)-4-((5-fluoro-4-(3-(2-oxopyridin-1(2H)-yl)phenyl)pyrimidin-2-yl)amino)-N-methylcyclohexane-1-carboxamide (Compound 488). To a solution of trans-4-[[5-fluoro-4-[3-(2-oxo-1-pyridyl)phenyl]pyrimidin-2-yl]amino]cyclohexanecarboxylic acid (80.7 mg, 198 μmol, 1.00 eq) in DMF (3 mL) were added DIEA (127.5 mg, 990 μmol, 172 μL, 5.00 eq) and HATU (52.6 mg, 138 μmol, 0.700 eq) at 25 °C, the mixture was stirred at 25 °C for 10 min. Then trans-3-[3-fluoro-4-[4-[4-(methylamino)cyclohexyl]piperazin-1-yl]anilino]piperidine-2,6-dione (147 mg, 277 μmol, 1.40 eq, TFA) was added at 45 °C, the mixture was stirred at 45 °C for 30 min. LCMS showed that the trans-4-[[5-fluoro-4-[3-(2-oxo-1-pyridyl)phenyl]pyrimidin-2-yl]amino]cyclohexanecarboxylic acid was consumed and the desired mass was detected. To the mixture was added H₂O (5 mL) and the mixture was extracted with EtOAc (5 mL*3). The combined organic layers were washed with saturated aqueous NaCl solution (5 mL), dried over Na₂SO₄, filtered and the filtrate was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 25%-55% B over 8.0 min) to give the title compound (Compound 488) (37.3 mg, 42.7 μmol, 21.6% yield, 92.4% purity) as a white solid. LCMS (ES⁺): m/z 808.4 [M+H].

### Example 462. Synthesis of trans-3-((3-fluoro-4-(4-((4-fluoro-1-(4-((5-fluoro-4-(3-(2-oxopyridin-1(2H)-yl)phenyl)pyrimidin-2-yl)amino)cyclohexane-1-carbonyl)piperidin-4-yl)methyl)piperazin-1-yl)phenyl)amino)piperidine-2,6-dione (Compound 528)

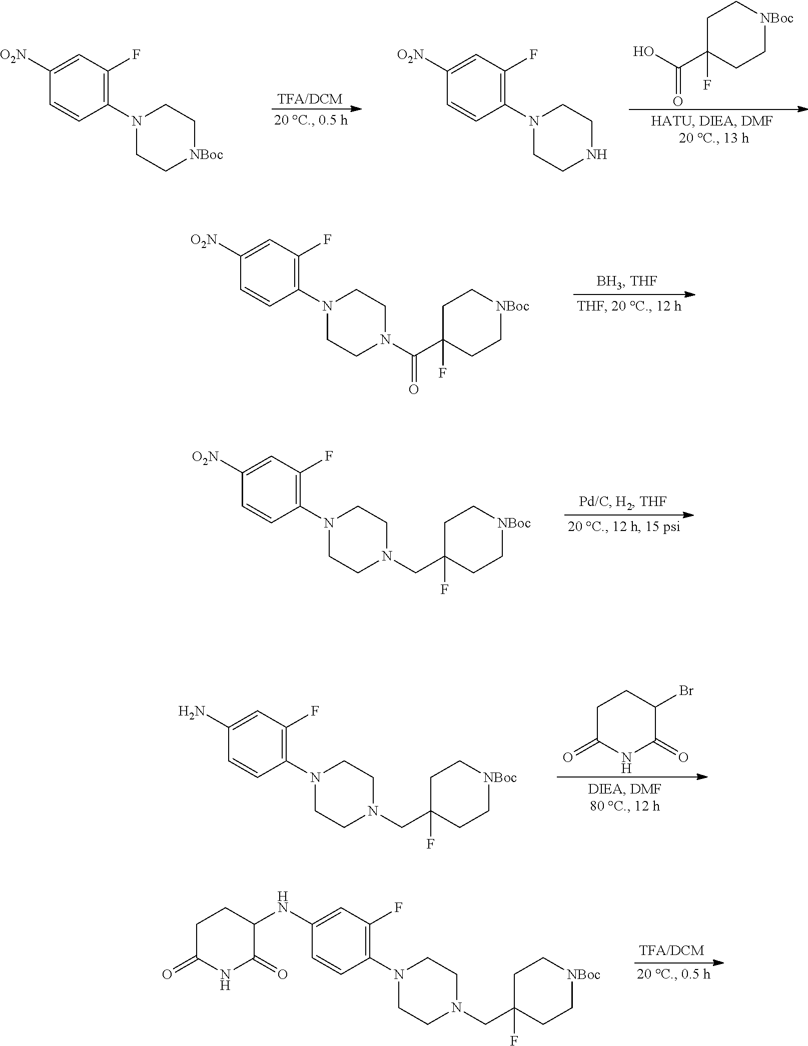

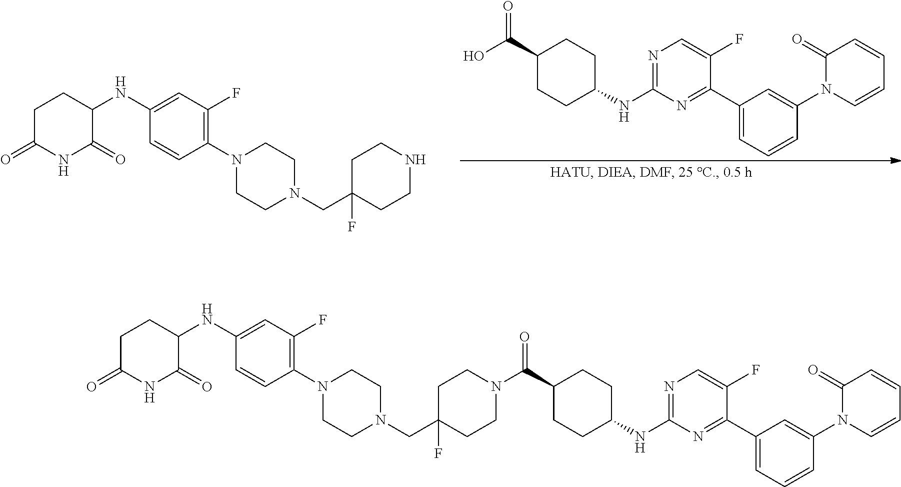 [SMILES: O=C1CCC(Nc2ccc(N3CCN(CC4(F)CCNCC4)CC3)c(F)c2)C(=O)N1 ; O=c1ccccn1-c1cccc(-c2nc(N[C@H]3CC[C@H](C(=O)O)CC3)ncc2F)c1 ; O=C1CCC(Nc2ccc(N3CCN(CC4(F)CCN(C(=O)[C@H]5CC[C@H](Nc6ncc(F)c(-c7cccc(-n8ccccc8=O)c7)n6)CC5)CC4)CC3)c(F)c2)C(=O)N1]

Synthesis of 1-(2-fluoro-4-nitrophenyl)piperazine. A solution of tert-butyl 4-(2-fluoro-4-nitrophenyl)piperazine-1-carboxylate (5.02 g, 15.4 mmol, 1.00 eq) in TFA (10 mL) and DCM (40 mL) was stirred at 20 °C for 0.5 h. LCMS showed tert-butyl 4-(2-fluoro-4-nitrophenyl)piperazine-1-carboxylate was consumed and the desired target MS was detected. The mixture was concentrated under reduced pressure to give the title compound (5.02 g, crude, TFA) as a yellow oil. LCMS (ES⁺): m/z 226.0 [M+H]⁺

Synthesis of tert-butyl 4-fluoro-4-(4-(2-fluoro-4-nitrophenyl)piperazine-1-carbonyl)piperidine-1-carboxylate. To a solution of 1-(tert-butoxycarbonyl)-4-fluoropiperidine-4-carboxylic acid (3.28 g, 13.3 mmol, 0.900 eq) in DMF (100 mL) were added HATU (3.36 g, 8.84 mmol, 0.600 eq), DIEA (9.52 g, 73.7 mmol, 12.8 mL, 5.00 eq) and 1-(2-fluoro-4-nitrophenyl)piperazine (5.00 g, 14.7 mmol, 1.00 eq, TFA) at 20 °C, then the mixture was stirred at 20 °C for 12 h. LCMS showed 1-(2-fluoro-4-nitrophenyl)piperazine remained little and the desired target MS was detected. Then 1-(tert-butoxycarbonyl)-4-fluoropiperidine-4-carboxylic acid (729 mg, 2.95 mmol, 0.200 eq) and HATU (1.12 g, 2.95 mmol, 0.200 eq) were added to the mixture at 20 °C and the mixture was stirred at 20 °C for another 1 h. LCMS showed 1-(2-fluoro-4-nitrophenyl)piperazine was consumed completely and the desired target MS was detected. H₂O (100 mL) was added to the solution, the mixture was extracted with EtOAc (100 mL*3), then the combined organic layers were washed with brine (100 mL*3), dried over Na₂SO₄, filtered and the filtrate was concentrated under reduced pressure to give a residue. The residue was purified by MPLC (SiO₂, PE/EtOAc=1/0 to 1/1) to give the title compound (4.70 g, 10.3 mmol, 70.2% yield) as a yellow solid. LCMS (ES⁺): m/z 399.1 [M+H−56]⁺

Synthesis of tert-butyl 4-fluoro-4-((4-(2-fluoro-4-nitrophenyl)piperazin-1-yl)methyl)piperidine-1-carboxylate. To a solution of tert-butyl 4-fluoro-4-(4-(2-fluoro-4-nitrophenyl)piperazine-1-carbonyl)piperidine-1-carboxylate (4.20 g, 9.24 mmol, 1.00 eq) in THF (50 mL) was added BH₃·THF (1 M, 55.5 mL, 6.00 eq) at 0 °C under N₂, then the mixture was stirred at 20 °C for 12 h under N₂. LCMS showed tert-butyl 4-fluoro-4-(4-(2-fluoro-4-nitrophenyl)piperazine-1-carbonyl)piperidine-1-carboxylate was consumed and the desired target MS was detected. MeOH (50 mL) was added dropwise to the mixture to quench the reaction and the mixture was stirred at 20 °C for 0.5 h. The mixture was concentrated under reduced pressure to give a residue. H₂O (50 mL) was added to the residue and it was extracted with EtOAc (50 mL*3). The combined organic layers were dried over Na₂SO₄, filtered and the filtrate was concentrated under reduced pressure to give a residue. The residue was purified by MPLC (SiO₂, PE/EtOAc=1/0 to 1/1) to give the title compound (3.20 g, 7.26 mmol, 78.6% yield) as a yellow solid. LCMS (ES⁺): m/z 441.2 [M+H]⁺

Synthesis of tert-butyl 4-((4-(4-amino-2-fluorophenyl)piperazin-1-yl)methyl)-4-fluoropiperidine-1-carboxylate. 10% Pd/C (3.20 g, 50.0% purity) was added to THF (80 mL) at 20 °C under N₂ atmosphere, then tert-butyl 4-fluoro-4-((4-(2-fluoro-4-nitrophenyl)piperazin-1-yl)methyl)piperidine-1-carboxylate (3.20 g, 7.26 mmol, 1.00 eq) was added to the mixture at 20 °C, the mixture was degassed and purged with H₂ for three times. Then the mixture was stirred at 20 °C for 12 h under H₂ (15 psi). LCMS showed tert-butyl 4-fluoro-4-((4-(2-fluoro-4-nitrophenyl)piperazin-1-yl)methyl)piperidine-1-carboxylate was consumed and the desired target MS was detected. The mixture were filtered and the filtrate was concentrated under reduced pressure to give the title compound (3.40 g, crude) as a yellow oil. LCMS (ES⁺): m/z 411.3 [M+H]⁺

Synthesis of tert-butyl 4-((4-(4-((2,6-dioxopiperidin-3-yl)amino)-2-fluorophenyl)piperazin-1-yl)methyl)-4-fluoropiperidine-1-carboxylate. To a solution of tert-butyl 4-((4-(4-amino-2-fluorophenyl)piperazin-1-yl)methyl)-4-fluoropiperidine-1-carboxylate (3.40 g, 8.28 mmol, 1.00 eq) and 3-bromopiperidine-2,6-dione (4.77 g, 24.9 mmol, 3.00 eq) in DMF (80 mL) was added DIEA (3.21 g, 24.9 mmol, 4.33 mL, 3.00 eq) at 20 °C, the mixture was stirred at 80 °C for 12 h. LCMS showed tert-butyl 4-((4-(4-amino-2-fluorophenyl)piperazin-1-yl)methyl)-4-fluoropiperidine-1-carboxylate was consumed mostly and the desired target MS was detected. To the residue was added H₂O (40 mL), the mixture was filtered and the filter cake was dried under reduced pressure to give the title compound (3.70 g, crude) as a black solid. LCMS (ES⁺): m/z 522.3 [M+H]⁺

Synthesis of 3-((3-fluoro-4-(4-((4-fluoropiperidin-4-yl)methyl)piperazin-1-yl)phenyl)amino)piperidine-2,6-dione. A solution of tert-butyl 4-((4-(4-((2,6-dioxopiperidin-3-yl)amino)-2-fluorophenyl)piperazin-1-yl)methyl)-4-fluoropiperidine-1-carboxylate (500 mg, 959 μmol, 1.00 eq) in DCM (6 mL) and TFA (2 mL) was stirred at 20 °C for 0.5 h. LCMS showed tert-butyl 4-((4-(4-((2,6-dioxopiperidin-3-yl)amino)-2-fluorophenyl)piperazin-1-yl)methyl)-4-fluoropiperidine-1-carboxylate was consumed and the desired target MS was detected. The mixture was concentrated under reduced pressure to give the title compound (500 mg, crude, TFA) as a brown oil. LCMS (ES⁺): m/z 422.2 [M+H]⁺

Synthesis of trans-3-((3-fluoro-4-(4-((4-fluoro-1-(4-((5-fluoro-4-(3-(2-oxopyridin-1(2H)-yl)phenyl)pyrimidin-2-yl)amino)cyclohexane-1-carbonyl)piperidin-4-yl)methyl)piperazin-1-yl)phenyl)amino)piperidine-2,6-dione (Compound 528). To a solution of trans-4-((5-fluoro-4-(3-(2-oxopyridin-1(2H)-yl)phenyl)pyrimidin-2-yl)amino)cyclohexane-1-carboxylic acid (229 mg, 560 μmol, 0.600 eq) in DMF (5 mL) were added DIEA (603 mg, 4.67 mmol, 813 μL, 5.00 eq) and HATU (124 mg, 327 μmol, 0.350 eq) at 25 °C, then to the mixture was added 3-((3-fluoro-4-(4-((4-fluoropiperidin-4-yl)methyl)piperazin-1-yl)phenyl)amino)piperidine-2,6-dione (500 mg, 934 μmol, 1.00 eq, TFA) at 25 °C. The resulting mixture was stirred at 25 °C for 0.5 h. LCMS showed 3-((3-fluoro-4-(4-((4-fluoropiperidin-4-yl)methyl)piperazin-1-yl)phenyl)amino)piperidine-2,6-dione was consumed and the desired target MS was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 35%-75% B over 8.0 min) to give the title compound (Compound 528) (94.9 mg, 116 μmol, 12.4% yield, 98.8% purity) as a yellow solid. LCMS (ES⁺): m/z 812.3 [M+H]⁺

### Example 463. Synthesis of trans-3-((3-fluoro-4-(1-((4-fluoro-1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)phenyl)amino)piperidine-2,6-dione (Compound 487)

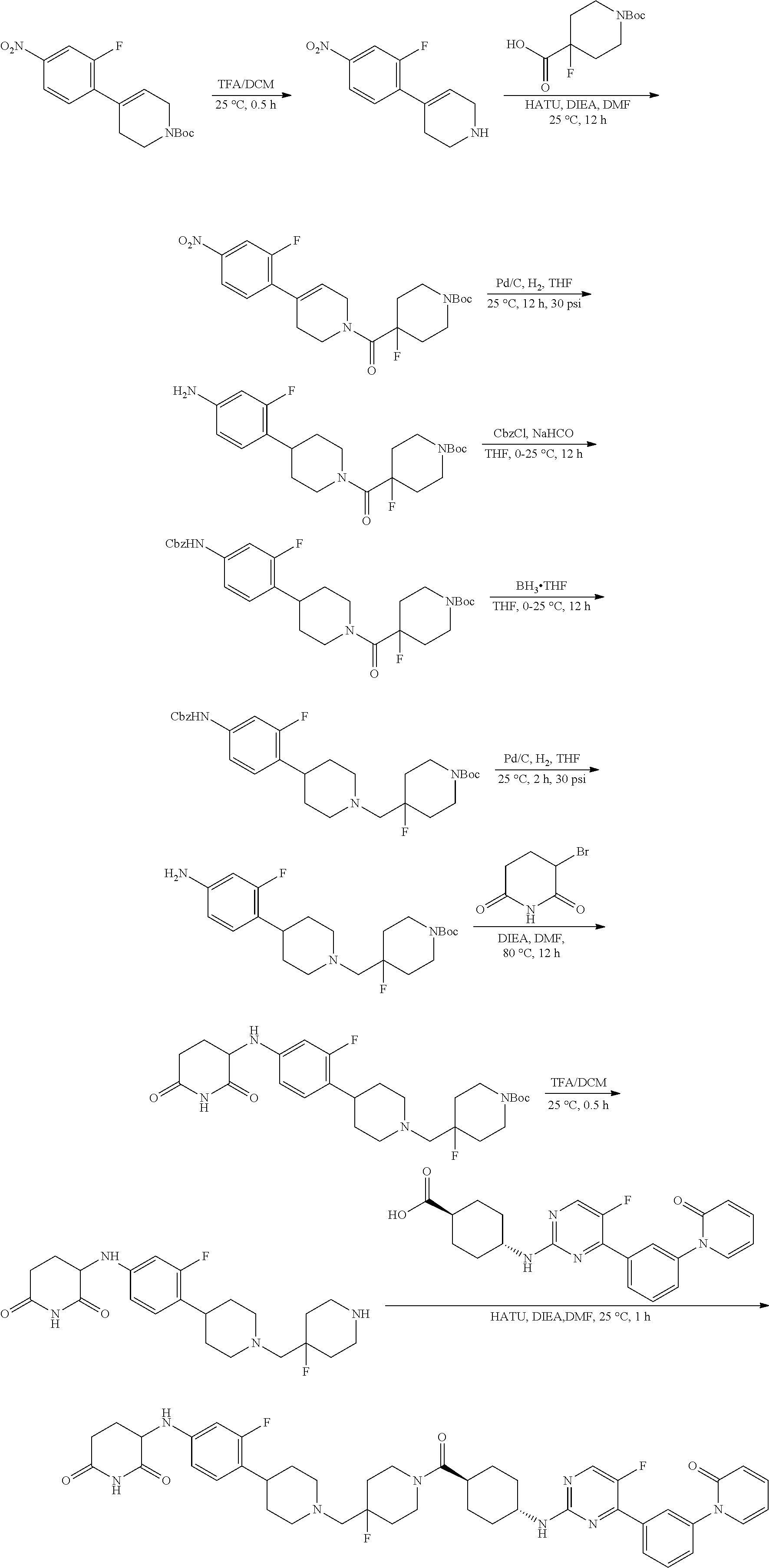

Synthesis of 4-(2-fluoro-4-nitrophenyl)-1,2,3,6-tetrahydropyridine. A solution of tert-butyl 4-(2-fluoro-4-nitrophenyl)-3,6-dihydropyridine-1(2H)-carboxylate (15.1 g, 46.9 mmol, 1.00 eq) in DCM (120 mL) and TFA (40 mL) was stirred at 25 °C for 0.5 h. LCMS showed tert-butyl 4-(2-fluoro-4-nitrophenyl)-3,6-dihydropyridine-1(2H)-carboxylate was consumed completely and the desired mass was detected. The mixture was concentrated under reduced pressure to give the title compound (15.1 g, crude, TFA) as a yellow oil. LCMS (ES⁺): m/z 223.1 [M+H]⁺.

Synthesis of tert-butyl 4-fluoro-4-(4-(2-fluoro-4-nitrophenyl)-1,2,3,6-tetrahydropyridine-1-carbonyl)piperidine-1-carboxylate. To a solution of 1-(tert-butoxycarbonyl)-4-fluoropiperidine-4-carboxylic acid (7.35 g, 29.7 mmol, 1.00 eq) in DMF (200 mL) were added HATU (17.0 g, 44.6 mmol, 1.50 eq) and DIEA (19.2 g, 149 mmol, 25.9 mL, 5.00 eq) at 25 °C, then to the reaction was added 4-(2-fluoro-4-nitrophenyl)-1,2,3,6-tetrahydropyridine (9.99 g, 29.7 mmol, 1.00 eq, TFA) at 25 °C. The resulting mixture was stirred at 25 °C for 12 h. LCMS showed 4-(2-fluoro-4-nitrophenyl)-1,2,3,6-tetrahydropyridine was consumed completely and the desired mass was detected. H₂O (300 mL) was added to the mixture and the mixture was extracted with EtOAc (150 ml*3). Then the combined organic layers were washed with brine (150 mL*3), dried over Na₂SO₄, filtered and the filtrate was concentrated under reduced pressure to give a residue. The residue was purified by MPLC (SiO₂, PE/EtOAc=1/0 to 2/1) to give the title compound (9.50 g, 21.0 mmol, 70.8% yield) as a red solid. LCMS (ES⁺): m/z 396.1 [M+H−56]⁺

Synthesis of tert-butyl 4-(4-(4-amino-2-fluorophenyl)piperidine-1-carbonyl)-4-fluoropiperidine-1-carboxylate. To 10% Pd/C (2.50 g, 50% purity) was added THF (50 mL) at 25 °C under N₂. Then tert-butyl 4-fluoro-4-(4-(2-fluoro-4-nitrophenyl)-1,2,3,6-tetrahydropyridine-1-carbonyl)piperidine-1-carboxylate (2.50 g, 5.54 mmol, 1.00 eq) was added to the mixture at 25 °C. The mixture was degassed and purged with H₂ for three times and the resulting mixture was stirred at 25 °C for 12 h under H₂ (30 psi). LCMS showed tert-butyl 4-fluoro-4-(4-(2-fluoro-4-nitrophenyl)-1,2,3,6-tetrahydropyridine-1-carbonyl)piperidine-1-carboxylate was consumed completely and the desired mass was detected. The mixture was filtered and the filtrate was concentrated under reduced pressure to give the title compound (2.46 g, crude) as a white solid. LCMS (ES⁺): m/z 324.1 [M+H−100]⁺.

Synthesis of tert-butyl 4-(4-(4-(((benzyloxy)carbonyl)amino)-2-fluorophenyl)piperidine-1-carbonyl)-4-fluoropiperidine-1-carboxylate. To a solution of tert-butyl 4-(4-(4-amino-2-fluorophenyl)piperidine-1-carbonyl)-4-fluoropiperidine-1-carboxylate (1.20 g, 2.83 mmol, 1.00 eq) and NaHCO₃ (476 mg, 5.67 mmol, 221 μL, 2.00 eq) in THF (20 mL) was added CbzCl (532 mg, 3.12 mmol, 445 μL, 1.10 eq) at 0 °C, the mixture was stirred at 25 °C for 12 h. LCMS showed tert-butyl 4-(4-(4-amino-2-fluorophenyl)piperidine-1-carbonyl)-4-fluoropiperidine-1-carboxylate was consumed completely and the desired mass was detected. The mixture was diluted with H₂O (30 mL) and then extracted with EtOAc (30 mL*3). The combined organic layers were dried over Na₂SO₄, filtered and the filtrate was concentrated under reduced pressure to give a residue. The residue was purified by MPLC (SiO₂, PE/EtOAc=1/0 to 5/1) to give the title compound (2.10 g, crude) as a white solid. After purification, the amount obtained exceeded the theoretical yield, so was treated as the crude product. LCMS (ES⁺): m/z 458.2 [M−100+H]⁺.

Synthesis of tert-butyl 4-((4-(4-(((benzyloxy)carbonyl)amino)-2-fluorophenyl)piperidin-1-yl)methyl)-4-fluoropiperidine-1-carboxylate. To a solution of tert-butyl 4-(4-(4-(((benzyloxy)carbonyl)amino)-2-fluorophenyl)piperidine-1-carbonyl)-4-fluoropiperidine-1-carboxylate (2.10 g, 3.77 mmol, 1.00 eq) in THF (30 mL) was added BH₃·THF (1 M, 16.1 mL, 9.00 eq) at 0 °C under N₂, the mixture was stirred at 25 °C for 12 h under N₂. LCMS showed tert-butyl 4-(4-(4-(((benzyloxy)carbonyl)amino)-2-fluorophenyl)piperidine-1-carbonyl)-4-fluoropiperidine-1-carboxylate was consumed completely and the desired mass was detected. The mixture is quenched by adding methanol (30 ml) dropwise until no air bubbles were formed. The mixture was diluted with H₂O (60 mL) and then extracted with EtOAc (60 mL*3). The combined organic layers were dried over Na₂SO₄, filtered and the filtrate was concentrated under reduced pressure to give a residue. The residue was purified by MPLC (SiO₂, DCM/EtOAc=1/0 to 10/1) to give the title compound (1.50 g, 2.76 mmol, 73.3% yield) as a white solid. LCMS (ES⁺): m/z 544.3 [M+H]⁺.

Synthesis of tert-butyl 4-((4-(4-amino-2-fluorophenyl)piperidin-1-yl)methyl)-4-fluoropiperidine-1-carboxylate. To 10% Pd/C (1.00 g, 50% purity) was added THF (30 mL) at 25 °C under N₂. Then tert-butyl 4-((4-(4-(((benzyloxy)carbonyl)amino)-2-fluorophenyl)piperidin-1-yl)methyl)-4-fluoropiperidine-1-carboxylate (500 mg, 920 μmol, 1.00 eq) was added to the mixture at 25 °C. The mixture was degassed and purged with H₂ for three times and the resulting mixture was stirred at 25 °C for 2 h under H₂ (30 psi). LCMS showed tert-butyl 4-((4-(4-(((benzyloxy)carbonyl)amino)-2-fluorophenyl)piperidin-1-yl)methyl)-4-fluoropiperidine-1-carboxylate was consumed completely and the desired mass was detected. The mixture was filtered, the filtrate was concentrated under reduced pressure to give the title compound (500 mg, crude) as a white solid. LCMS (ES⁺): m/z 410.2 [M+H]⁺.

Synthesis of tert-butyl 4-((4-(4-((2,6-dioxopiperidin-3-yl)amino)-2-fluorophenyl)piperidin-1-yl)methyl)-4-fluoropiperidine-1-carboxylate. To a solution of tert-butyl 4-((4-(4-amino-2-fluorophenyl)piperidin-1-yl)methyl)-4-fluoropiperidine-1-carboxylate (500 mg, 1.22 μmol, 1.00 eq) in DMF (10 mL) were added DIEA (316 mg, 2.44 mmol, 425 L, 2.00 eq) and 3-bromopiperidine-2,6-dione (352 mg, 1.83 μmol, 1.50 eq) at 25 °C, the mixture was stirred at 80 °C for 12 h. LCMS showed tert-butyl 4-((4-(4-amino-2-fluorophenyl)piperidin-1-yl)methyl)-4-fluoropiperidine-1-carboxylate was consumed completely and the desired mass was detected. H₂O (20 mL) was added to the solution and the mixture was extracted with EtOAc (20 mL*3). Then the combined organic layers were washed with brine (20 mL*3), dried over Na₂SO₄, filtered and the filtrate was concentrated under reduced pressure to give a residue. The residue was purified by prep-TLC (SiO₂, DCM/EtOAc=10/1) to give the title compound (150 mg, 288 μmol, 23.6% yield) as a green oil. LCMS (ES⁺): m/z 521.3 [M+H]⁺.

Synthesis of 3-((3-fluoro-4-(1-((4-fluoropiperidin-4-yl)methyl)piperidin-4-yl)phenyl)amino)piperidine-2,6-dione. A solution of tert-butyl 4-((4-(4-((2,6-dioxopiperidin-3-yl)amino)-2-fluorophenyl)piperidin-1-yl)methyl)-4-fluoropiperidine-1-carboxylate (150 mg, 288 μmol, 1.00 eq) in DCM (3 mL) and TFA (1 mL) was stirred at 25 °C for 0.5 h. LCMS showed tert-butyl 4-((4-(4-((2,6-dioxopiperidin-3-yl)amino)-2-fluorophenyl)piperidin-1-yl)methyl)-4-fluoropiperidine-1-carboxylate was consumed completely and the desired mass was detected. The mixture was concentrated under reduced pressure to give the title compound (150 mg, crude, TFA) as a yellow oil. LCMS (ES⁺): m/z 421.3 [M+H]⁺.

Synthesis of trans-3-((3-fluoro-4-(1-((4-fluoro-1-(4-((5-fluoro-4-(3-(2-oxopyridin-1(2H)-yl)phenyl)pyrimidin-2-yl)amino)cyclohexane-1-carbonyl)piperidin-4-yl)methyl)piperidin-4-yl)phenyl)amino)piperidine-2,6-dione (Compound 487). To a solution of trans-4-((5-fluoro-4-(3-(2-oxopyridin-1(2H)-yl)phenyl)pyrimidin-2-yl)amino)cyclohexane-1-carboxylic acid (68.8 mg, 168 μmol, 0.600 eq) in DMF (3 mL) were added HATU (64.0 mg, 168 μmol, 0.600 eq) and DIEA (182 mg, 1.41 mmol, 245 μL, 5.00 eq) at 25 °C, then to the mixture was added 3-((3-fluoro-4-(1-((4-fluoropiperidin-4-yl)methyl)piperidin-4-yl)phenyl)amino)piperidine-2,6-dione (150 mg, 281 μmol, 1.00 eq, TFA) at 25 °C, the mixture was stirred at 25 °C for 1 h. LCMS showed 3-((3-fluoro-4-(1-((4-fluoropiperidin-4-yl)methyl)piperidin-4-yl)phenyl)amino)piperidine-2,6-dione was consumed completely and the desired mass was detected. The mixture was concentrated under reduced pressure to give a residue. The residue was purified by prep-HPLC (column: Phenomenex luna C18 100*40 mm*3 um; mobile phase: [1H₂O (0.2% FA)-ACN]; gradient: 15%-55% B over 8.0 min) to give the title compound (Compound 487) (39.3 mg, 47.0 μmol, 16.7% yield, 96.9% purity) as a white solid. LCMS (ES⁺): m/z 811.3 [M+H]⁺

### Example 464. Synthesis of 1′-(4-((2,6-dioxopiperidin-3-yl)amino)-2-fluorophenyl)-3′-fluoro-N-((1r,4r)-4-((5-fluoro-4-(3-(2-oxopyridin-1(2H)-yl)phenyl)pyrimidin-2-yl)amino)cyclohexyl)-[1,4′-bipiperidine]-4-carboxamide (Compound 405)

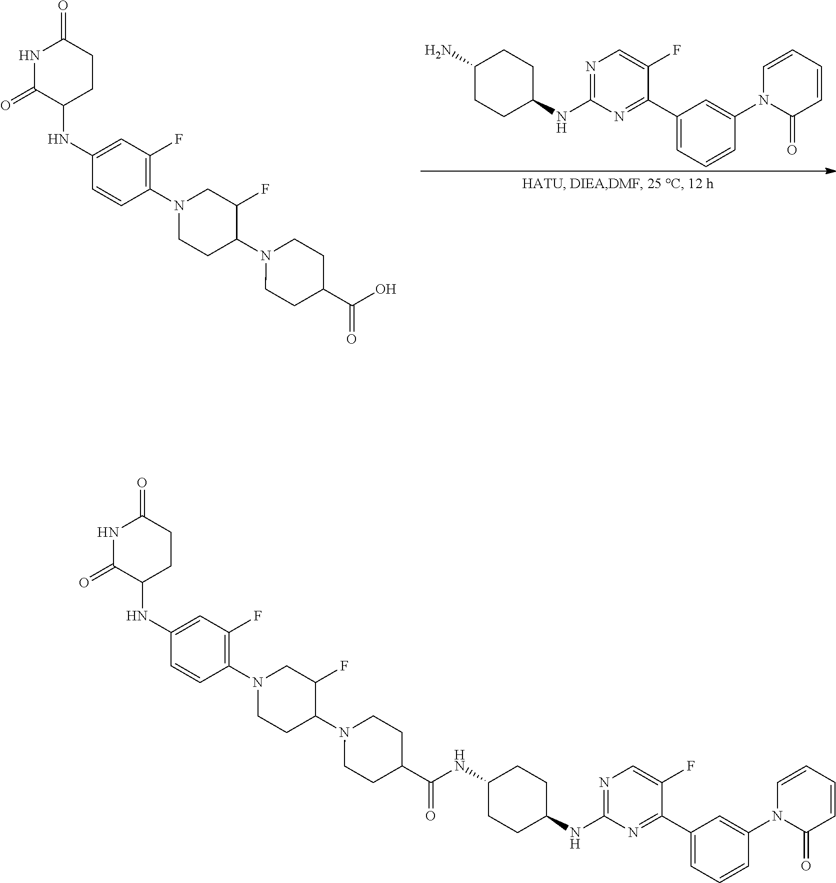 [SMILES: O=C1CCC(Nc2ccc(N3CCC(N4CCC(C(=O)O)CC4)C(F)C3)c(F)c2)C(=O)N1 ; N[C@H]1CC[C@H](Nc2ncc(F)c(-c3cccc(-n4ccccc4=O)c3)n2)CC1 ; O=C1CCC(Nc2ccc(N3CCC(N4CCC(C(=O)N[C@H]5CC[C@H](Nc6ncc(F)c(-c7cccc(-n8ccccc8=O)c7)n6)CC5)CC4)C(F)C3)c(F)c2)C(=O)N1]

Synthesis of 1′-(4-((2,6-dioxopiperidin-3-yl)amino)-2-fluorophenyl)-3′-fluoro-N-((1r,4r)-4-((5-fluoro-4-(3-(2-oxopyridin-1(2H)-yl)phenyl)pyrimidin-2-yl)amino)cyclohexyl)-[1,4′-bipiperidine]-4-carboxamide (Compound 405). To a solution of 1′-(4-((2,6-dioxopiperidin-3-yl)amino)-2-fluorophenyl)-3′-fluoro-[1,4′-bipiperidine]-4-carboxylic acid (1.00 g, 2.22 mmol, 1.00 eq) in DMF (15 mL) were added HATU (675 mg, 1.78 mmol, 0.800 eq) and DIEA (1.43 g, 11.1 mmol, 1.93 mL, 5.00 eq) at 25 °C, to the reaction was added trans-1-(3-(2-((4-aminocyclohexyl)amino)-5-fluoropyrimidin-4-yl)phenyl)pyridin-2(1H)-one (876 mg, 1.78 mmol, 0.800 eq, TFA) at 25 °C, the mixture was stirred at 25 °C for 12 h. LCMS showed 1′-(4-((2,6-dioxopiperidin-3-yl)amino)-2-fluorophenyl)-3′-fluoro-[1,4′-bipiperidine]-4-carboxylic acid was consumed completely and the desired mass was detected. The mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 10%-45% B over 8.0 min) to give the title compound (Compound 405) (110 mg, 130 μmol, 5.87% yield, 96.0% purity) as a yellow solid. LCMS (ES⁺): m/z 812.3 [M+H]⁺

### Example 465. Synthesis of trans-1′-(4-((2,6-dioxopiperidin-3-yl)amino)-2-fluorophenyl)-N-(4-((5-fluoro-4-(3-(2-oxopyridin-1(2H)-yl)phenyl)pyrimidin-2-yl)amino)cyclohexyl)-4-hydroxy-[1,4′-bipiperidine]-4-carboxamide (Compound 444)

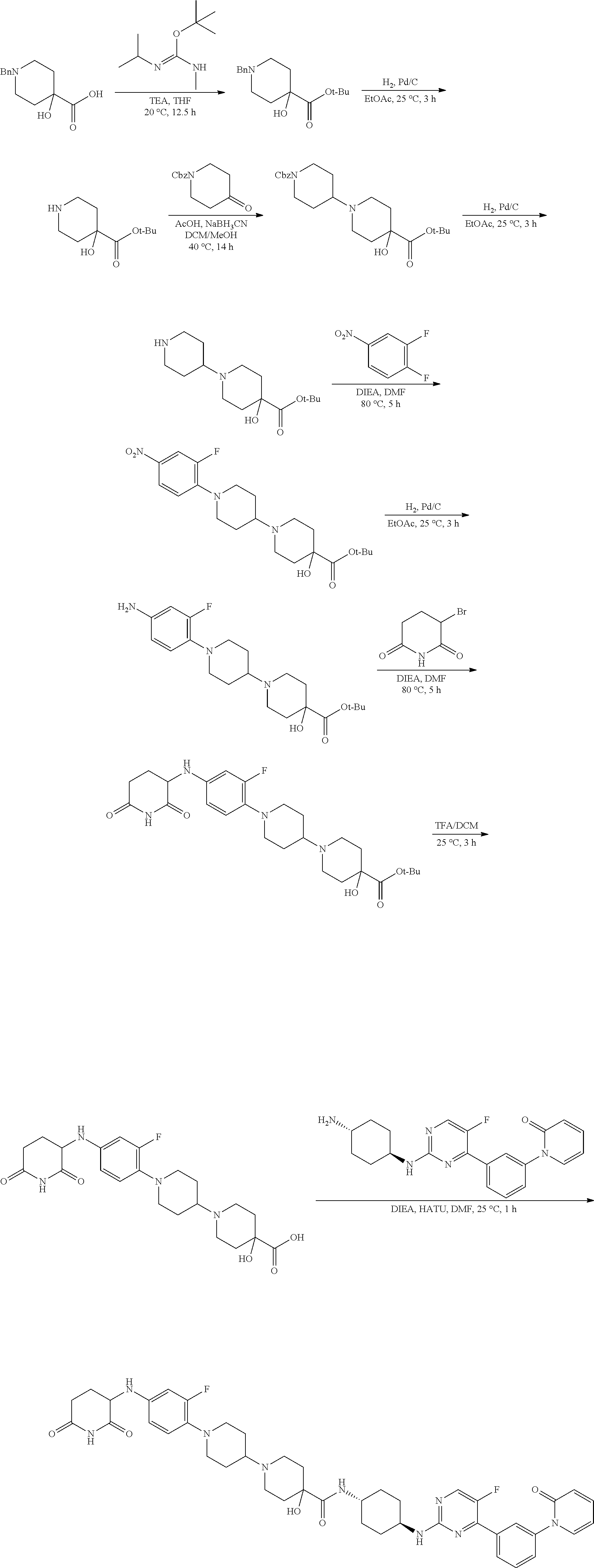

Synthesis of tert-butyl 1-benzyl-4-hydroxypiperidine-4-carboxylate. To a solution of 1-benzyl-4-hydroxypiperidine-4-carboxylic acid (10.0 g, 42.5 mmol, 1.00 eq) in THE (300 mL) was added TEA (8.60 g, 85.0 mmol, 11.8 mL, 2.00 eq) at 20 °C. The mixture was stirred at 20 °C for 30 min. Then tert-butyl (Z)—N,N′-diisopropylcarbamimidate (51.1 g, 255 mmol, 6.00 eq) was added to the mixture at 20 °C. The mixture was stirred at 20 °C for 12 h. LC-MS showed 1-benzyl-4-hydroxypiperidine-4-carboxylic acid was remaining little and the desired mass was detected. Water (200 mL) was added to the mixture at 20 °C. Then the reaction mixture was extracted with DCM (300 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=5:1 to 2:1) to give the title Compound (5.00 g, 17.2 mmol, 40.4% yield) as a white solid. LCMS (ES⁺): m/z 292.2 [M+H]⁺.

Synthesis of tert-butyl 4-hydroxypiperidine-4-carboxylate. 10% Pd/C (3.00 g, 50% purity) was added to EtOAc (70 mL) at 25 °C under N₂ atmosphere, then tert-butyl 1-benzyl-4-hydroxypiperidine-4-carboxylate (4.50 g, 15.4 mmol, 1.00 eq) was added to the mixture at 25 °C, the suspension was degassed and purged with H₂ for three times. The mixture was stirred at 25 °C for 4 h under H₂ (15 psi) atmosphere. LC-MS showed tert-butyl 1-benzyl-4-hydroxypiperidine-4-carboxylate was consumed completely and the desired mass was detected. The product was filtered and the filtrate was concentrated under reduced pressure to give the title Compound (3.10 g, crude) as a brown oil. LCMS (ES⁺): m/z 201.9 [M+H]⁺.

Synthesis of 1′-benzyl 4-(tert-butyl) 4-hydroxy-[1,4′-bipiperidine]-1′,4-dicarboxylate. To a solution of tert-butyl 4-hydroxypiperidine-4-carboxylate (3.10 g, 15.4 mmol, 1.00 eq) and benzyl 4-oxopiperidine-1-carboxylate (21.6 g, 92.4 mmol, 18.4 mL, 6.00 eq) in DCM (100 mL) and MeOH (100 mL) was added AcOH (92.5 mg, 1.54 mmol, 88.2 μL, 0.1 eq) at 20 °C, the mixture was stirred at 40 °C for 2 h, then NaBH₃CN (2.90 g, 46.2 mmol, 3.00 eq) was added to the mixture, the mixture was stirred at 40 °C for 12 h. LCMS showed tert-butyl 4-hydroxypiperidine-4-carboxylate was consumed completely and the desired mass was detected. The reaction was concentrated under reduced pressure to give a residue. Then water (100 mL) was added to the residue, then it was extracted with EtOAc (50 mL*3), the combined organic layers were dried over Na₂SO₄, filtered and the filtrate was concentrated under reduced pressure to give a residue. The residue was purified by column chromatography (SiO₂, Petroleum ether:Ethyl acetate=1:0 to 0:1) to give the title compound (4.20 g, 10.0 mmol, 65.2% yield) as a yellow oil. LCMS (ES⁺): m/z 419.3 [M+H]⁺.

Synthesis tert-butyl 4-hydroxy-[1,4′-bipiperidine]-4-carboxylate. 10% Pd/C (5.00 g, 50% purity) was added to EtOAc (60 mL) at 25 °C under N₂ atmosphere, then 1′-benzyl 4-(tert-butyl) 4-hydroxy-[1,4′-bipiperidine]-1′,4-dicarboxylate (3.90 g, 9.32 mmol, 1.00 eq) was added to the mixture at 25 °C, the suspension was degassed and purged with H₂ for three times. The mixture was stirred at 25 °C for 2 h under H₂ (15 psi) atmosphere. LC-MS showed 1′-benzyl 4-(tert-butyl) 4-hydroxy-[1,4′-bipiperidine]-1′,4-dicarboxylate was consumed completely and the desired mass was detected. The mixture was filtered and the filtrate was concentrated under reduced pressure to give the title Compound (2.65 g, crude) as a brown solid. LCMS (ES⁺): m/z 285.3 [M+H]⁺.

Synthesis of tert-butyl 1′-(2-fluoro-4-nitrophenyl)-4-hydroxy-[1,4′-bipiperidine]-4-carboxylate. To a solution of tert-butyl 4-hydroxy-[1,4′-bipiperidine]-4-carboxylate (1.97 g, 6.93 mmol, 1.00 eq) in DMF (20 mL) were added DIEA (1.79 g, 13.9 mmol, 2.41 mL, 2.00 eq) and 1,2-difluoro-4-nitrobenzene (1.10 g, 6.93 mmol, 767 μL, 1.00 eq) at 25 °C. The mixture was stirred at 80 °C for 12 h. LC-MS showed tert-butyl 4-hydroxy-[1,4′-bipiperidine]-4-carboxylate was consumed completely and the desired mass was detected. Water (20 mL) was added to the mixture at 25 °C. Then the reaction mixture was extracted with EtOAc (10 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=3:1 to 1:1) to give the title compound (1.59 g, 3.75 mmol, 54.2% yield) as a yellow solid. LCMS (ES⁺): m/z 424.2 [M+H]⁺.

Synthesis of tert-butyl 1′-(4-amino-2-fluorophenyl)-4-hydroxy-[1,4′-bipiperidine]-4-carboxylate. 10% Pd/C (2.00 g, 50% purity) was added to EtOAc (40 mL) at 25 °C under N₂ atmosphere, then tert-butyl 1′-(2-fluoro-4-nitrophenyl)-4-hydroxy-[1,4′-bipiperidine]-4-carboxylate (1.59 g, 3.75 mmol, 1.00 eq) was added to the mixture at 25 °C, the suspension was degassed and purged with H₂ for three times. The mixture was stirred at 25 °C for 3 h under H₂ (15 psi) atmosphere. LC-MS showed tert-butyl 1′-(2-fluoro-4-nitrophenyl)-4-hydroxy-[1,4′-bipiperidine]-4-carboxylate was consumed completely and the desired mass was detected. The mixture was filtered and the filtrate was concentrated under reduced pressure to give the title compound (1.60 g, crude) as a brown solid. LCMS (ES⁺): m/z 394.2 [M+H]⁺.

Synthesis of tert-butyl 1′-(4-((2,6-dioxopiperidin-3-yl)amino)-2-fluorophenyl)-4-hydroxy-[1,4′-bipiperidine]-4-carboxylate. To a solution of tert-butyl 1′-(4-amino-2-fluorophenyl)-4-hydroxy-[1,4′-bipiperidine]-4-carboxylate (1.40 g, 3.56 mmol, 1.00 eq) in DMF (30 mL) were added DIEA (920 mg, 7.12 mmol, 1.24 mL, 2.00 eq) and 3-bromopiperidine-2,6-dione (1.71 g, 8.89 mmol, 2.50 eq) at 25 °C. The mixture was stirred at 80 °C for 3 h. LC-MS showed tert-butyl 1′-(4-amino-2-fluorophenyl)-4-hydroxy-[1,4′-bipiperidine]-4-carboxylate was remaining and the desired mass was detected. Then 3-bromopiperidine-2,6-dione (342 mg, 1.78 mmol, 0.500 eq) was added to the mixture at 25 °C. The mixture was stirred at 80 °C for another 2 h. LC-MS showed tert-butyl 1′-(4-amino-2-fluorophenyl)-4-hydroxy-[1,4′-bipiperidine]-4-carboxylate was consumed completely and the desired mass was detected. Water (20 mL) was added to the mixture at 25 °C. Then the reaction mixture was extracted with EtOAc (10 mL*3). The organic layers were dried over anhydrous sodium sulfate, filtered and the filtrate was concentrated under reduced pressure to give a residue. The residue was purified by prep-TLC (SiO₂, Petroleum ether:Ethyl acetate=1:1) to give the title compound (1.50 g, 2.97 mmol, 83.6% yield) as a brown solid. LCMS (ES⁺): m/z 505.2 [M+H]⁺.

Synthesis of 1′-(4-((2,6-dioxopiperidin-3-yl)amino)-2-fluorophenyl)-4-hydroxy-[1,4′-bipiperidine]-4-carboxylic acid. To a solution of tert-butyl 1′-(4-((2,6-dioxopiperidin-3-yl)amino)-2-fluorophenyl)-4-hydroxy-[1,4′-bipiperidine]-4-carboxylate (1.20 g, 2.38 mmol, 1.00 eq) in DCM (6 mL) was added TFA (3.07 g, 26.9 mmol, 2 mL, 11.3 eq) at 25 °C. The mixture was stirred at 25 °C for 3 h. LC-MS showed tert-butyl 1′-(4-((2,6-dioxopiperidin-3-yl)amino)-2-fluorophenyl)-4-hydroxy-[1,4′-bipiperidine]-4-carboxylate was consumed completely and the desired mass was detected. The reaction mixture was concentrated under reduced pressure to remove solvent to give the title compound (1.07 g, crude) as a yellow oil. LCMS (ES⁺): m/z 449.2 [M+H]⁺.

Synthesis of trans-1′-(4-((2,6-dioxopiperidin-3-yl)amino)-2-fluorophenyl)-N-(4-((5-fluoro-4-(3-(2-oxopyridin-1(2H)-yl)phenyl)pyrimidin-2-yl)amino)cyclohexyl)-4-hydroxy-[1,4′-bipiperidine]-4-carboxamide (Compound 444). To a solution of 1′-(4-((2,6-dioxopiperidin-3-yl)amino)-2-fluorophenyl)-4-hydroxy-[1,4′-bipiperidine]-4-carboxylic acid (1.07 g, 2.39 mmol, 1.00 eq) in DMF (20 mL) were added trans-1-(3-(2-(((1r,4r)-4-aminocyclohexyl)amino)-5-fluoropyrimidin-4-yl)phenyl)pyridin-2(1H)-one (471 mg, 954 μmol, 0.400 eq, TFA), HATU (726 mg, 1.91 mmol, 0.800 eq) and DIEA (1.54 g, 12.0 mmol, 2.09 mL, 5.00 eq) at 25 °C. The mixture was stirred at 25 °C for 1 h. LC-MS showed 1′-(4-((2,6-dioxopiperidin-3-yl)amino)-2-fluorophenyl)-4-hydroxy-[1,4′-bipiperidine]-4-carboxylic acid was consumed completely and the desired mass was detected. The reaction mixture was concentrated under reduced pressure to give a residue. The residue was purified by prep-HPLC (column: Waters Xbridge BEH C18 250*50 mm*10 um; mobile phase: [H₂O (10 mM NH₄HCO₃)-ACN]; gradient: 20%-50% B over 10.0 min) to give the title compound (Compound 444) (96.3 mg, 110 μmol, 4.62% yield, 92.7% purity) as a yellow solid. LCMS (ES⁺): m/z 810.4 [M+H]⁺

### Example 466. Synthesis of trans-(R)-1′-(4-((2,6-dioxopiperidin-3-yl)amino)-2-fluorophenyl)-N-(4-((5-fluoro-4-(3-(2-oxopyridin-1(2H)-yl)phenyl)pyrimidin-2-yl)amino)cyclohexyl)-4-hydroxy-[1,4′-bipiperidine]-4-carboxamide (Compound 595) and trans-(S)-1′-(4-((2,6-dioxopiperidin-3-yl)amino)-2-fluorophenyl)-N-(4-((5-fluoro-4-(3-(2-oxopyridin-1(2H)-yl)phenyl)pyrimidin-2-yl)amino)cyclohexyl)-4-hydroxy-[1,4′-bipiperidine]-4-carboxamide (Compound 594) (The Two Isomers were Assigned Arbitrarily)

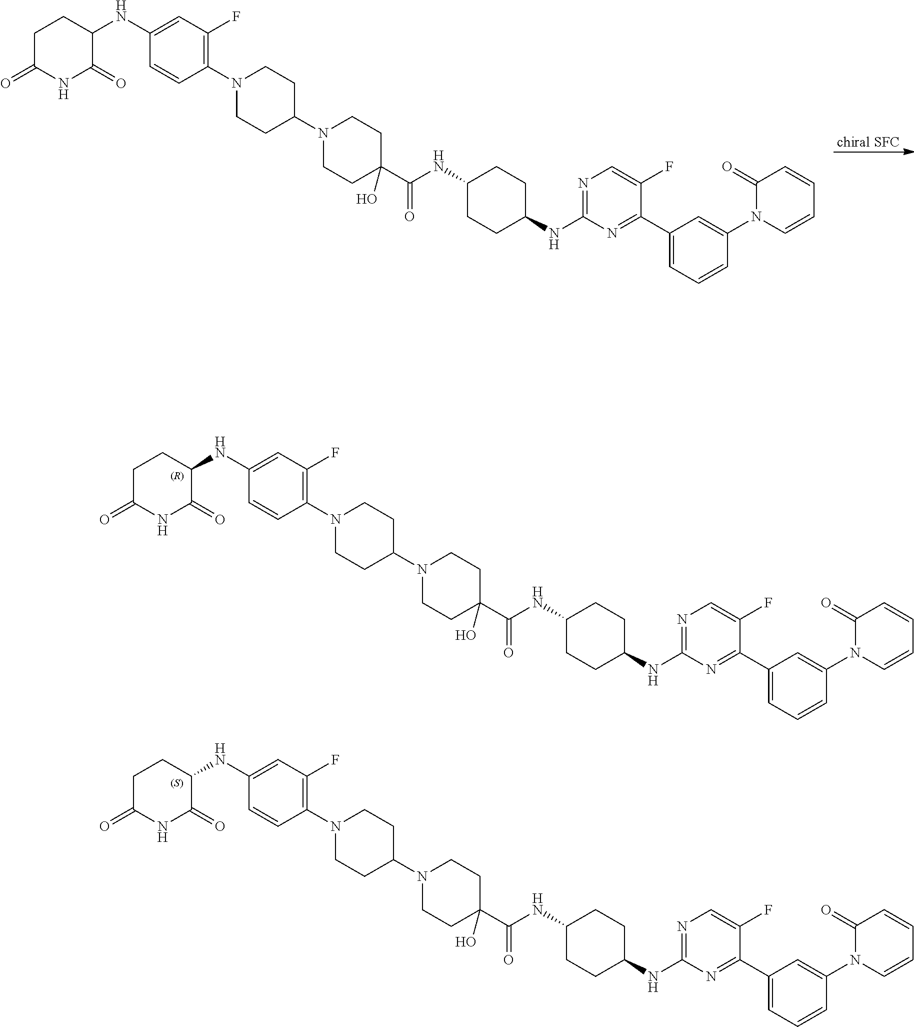

Synthesis of trans-(R)-1′-(4-((2,6-dioxopiperidin-3-yl)amino)-2-fluorophenyl)-N-(4-((5-fluoro-4-(3-(2-oxopyridin-1(2H)-yl)phenyl)pyrimidin-2-yl)amino)cyclohexyl)-4-hydroxy-[1,4′-bipiperidine]-4-carboxamide (Compound 595) and trans-(S)-1′-(4-((2,6-dioxopiperidin-3-yl)amino)-2-fluorophenyl)-N-(4-((5-fluoro-4-(3-(2-oxopyridin-1(2H)-yl)phenyl)pyrimidin-2-yl)amino)cyclohexyl)-4-hydroxy-[1,4′-bipiperidine]-4-carboxamide (Compound 594) (the two isomers were assigned arbitrarily). Trans-1′-(4-((2,6-dioxopiperidin-3-yl)amino)-2-fluorophenyl)-N-(4-((5-fluoro-4-(3-(2-oxopyridin-1(2H)-yl)phenyl)pyrimidin-2-yl)amino)cyclohexyl)-4-hydroxy-[1,4′-bipiperidine]-4-carboxamide (Compound 444) (90.0 mg, 111 μmol, 92.7% purity) was further separated by SFC (column: DAICEL CHIRALPAK IE (250 mm*30 mm, 10 um); mobile phase: [Heptane-IPA:ACN=4:1]; B %: 100%, isocratic elution mode) to give the title compound (Compound 595) (17.6 mg, 21.7 μmol, 19.6% yield, 100% purity, 97.4% ee) (retention time: 2.97, peak 1) as an off-white solid. LCMS (ES⁺): m/z 810.5 [M+H]⁺ and give the title compound (Compound 594) (15.2 mg, 17.6 μmol, 15.9% yield, 94.0% purity, 97.2% ee) (retention time: 4.38, peak 2) as a white solid. LCMS (ES⁺): m/z 810.4 [M+H]⁺

### Example 467. Synthesis of trans-4-(4-(3-(2,4-dioxotetrahydropyrimidin-1(2H)-yl)-5-fluoro-1-methyl-1H-indazol-6-yl)piperidin-1-yl)-N-(trans-4-((5-fluoro-4-(3-(2-oxopyridin-1(2H)-yl)phenyl)pyrimidin-2-yl)amino)cyclohexyl)cyclohexane-1-carboxamide (Compound 512)

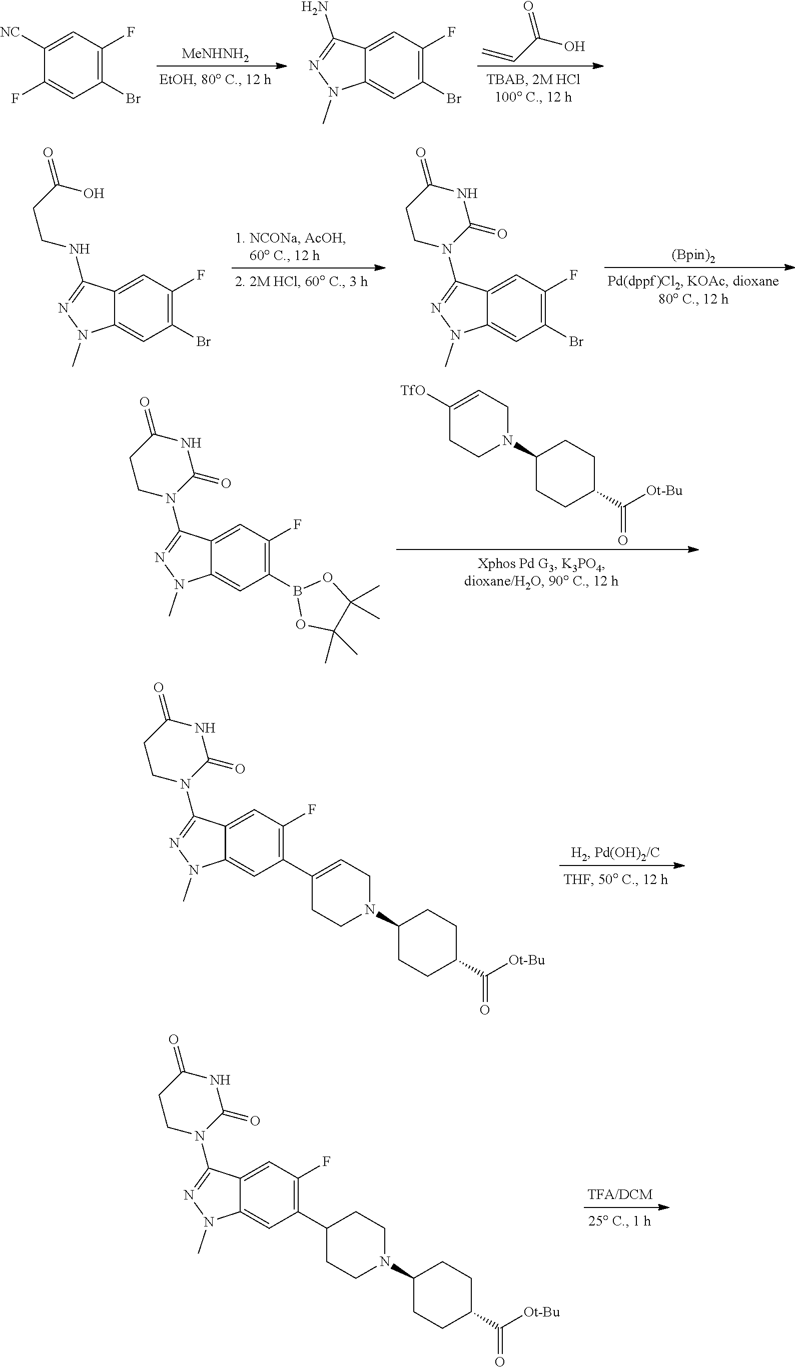

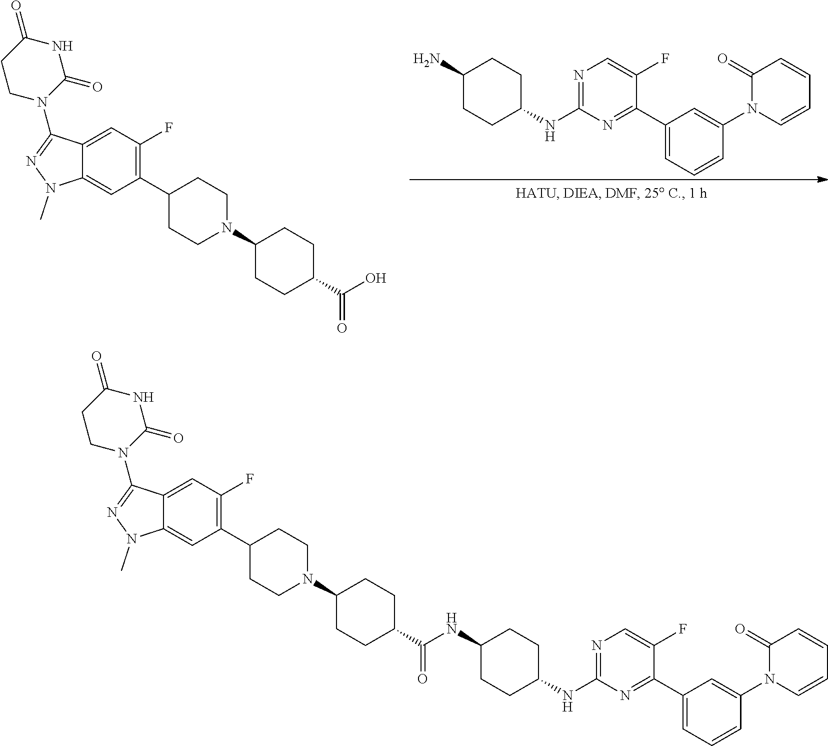 [SMILES: Cn1nc(N2CCC(=O)NC2=O)c2cc(F)c(C3CCN([C@H]4CC[C@H](C(=O)O)CC4)CC3)cc21 ; N[C@H]1CC[C@H](Nc2ncc(F)c(-c3cccc(-n4ccccc4=O)c3)n2)CC1 ; Cn1nc(N2CCC(=O)NC2=O)c2cc(F)c(C3CCN([C@H]4CC[C@H](C(=O)N[C@H]5CC[C@H](Nc6ncc(F)c(-c7cccc(-n8ccccc8=O)c7)n6)CC5)CC4)CC3)cc21]

Synthesis of 6-bromo-5-fluoro-1-methyl-1H-indazol-3-amine. To a solution of 4-bromo-2,5-difluorobenzonitrile (25.0 g, 115 mmol, 1.00 eq) in EtOH (250 mL) was added CH₃NHNH₂ (46.9 g, 407 mmol, 53.6 mL, 3.55 eq) at 15 °C. The mixture was stirred at 80 °C for 12 h. LC-MS showed that 4-bromo-2,5-difluorobenzonitrile was consumed completely, and the desired mass was detected. The reaction mixture was concentrated under reduced pressure. The residue was purified by column chromatography (SiO₂, Petroleum ether/Ethyl acetate=3/1 to 1/1) to give title Compound (25.0 g, 102 mmol, 89.3% yield) as a yellow solid. LCMS (ES⁺): m/z 244.0 [M+H]⁺.

Synthesis of 3-((6-bromo-5-fluoro-1-methyl-1H-indazol-3-yl)amino)propanoic acid. To a solution of 6-bromo-5-fluoro-1-methyl-1H-indazol-3-amine (20.0 g, 81.9 mmol, 1.00 eq) in HCl (2.00 M, 200 mL, 4.88 eq) was added TBAB (2.64 g, 8.19 mmol, 0.100 eq) and acrylic acid (8.86 g, 123 mmol, 8.43 mL, 1.50 eq) at 25 °C. The mixture was stirred at 100 °C for 12 h. LC-MS showed that 6-bromo-5-fluoro-1-methyl-1H-indazol-3-amine was consumed completely and the desired mass was detected. The reaction mixture was added saturated solution of NaHCO₃ to adjust pH=8. Then the mixture was acidified with acetic acid to adjust pH=5. The reaction mixture was filtered and the filtrate was concentrated under reduced pressure to give title Compound (20.0 g, crude) as a yellow solid. LCMS (ES⁺): m/z 316.0 [M+H]⁺.

Synthesis of 1-(6-bromo-5-fluoro-1-methyl-1H-indazol-3-yl)dihydropyrimidine-2,4(1H,3H)-dione. To a solution of 3-((6-bromo-5-fluoro-1-methyl-1H-indazol-3-yl)amino)propanoic acid (20.0 g, 63.2 mmol, 1.00 eq) in AcOH (200 mL) was added NaOCN (8.74 g, 127 mmol, 2.00 eq) at 15 °C. The mixture was stirred at 60 °C for 12 h. Then HCl (2.00 M, 200 mL, 6.32 eq) was added at 15 °C. The mixture was stirred at 60 °C for 3 h. LC-MS showed that 3-((6-bromo-5-fluoro-1-methyl-1H-indazol-3-yl)amino)propanoic acid was consumed completely and the desired mass was detected. The reaction mixture was filtered and the filtrate was concentrated under reduced pressure to give title Compound (9.00 g, crude) as a yellow solid. LCMS (ES⁺): m/z 341.0 [M+H]⁺.

Synthesis of 1-(5-fluoro-1-methyl-6-(4,4,5,5-tetramethyl-1,3,2-dioxaborolan-2-yl)-1H-indazol-3-yl)dihydropyrimidine-2,4(1H,3H)-dione. To a solution of 1-(6-bromo-5-fluoro-1-methyl-1H-indazol-3-yl)dihydropyrimidine-2,4(1H,3H)-dione (8.80 g, 25.8 mmol, 1.00 eq) in dioxane (200 mL) was added 4,4,5,5-tetramethyl-2-(4,4,5,5-tetramethyl-1,3,2-dioxaborolan-2-yl)-1,3,2-dioxaborolane (13.1 g, 51.6 mmol, 2.00 eq), KOAc (7.60 g, 77.4 mmol, 3.00 eq) and Pd(dppf)Cl₂ (1.89 g, 2.58 mmol, 0.100 eq) at 15 °C. The mixture was stirred at 80 °C for 12 h under N₂. LC-MS showed that 1-(6-bromo-5-fluoro-1-methyl-1H-indazol-3-yl)dihydropyrimidine-2,4(1H,3H)-dione was consumed completely and the desired mass was detected. The reaction mixture was diluted with water (100 mL) and extracted with EtOAc (100 mL*3). The combined organic layers were washed with brine (100 mL*3), dried over anhydrous Na₂SO₄, filtered and the filtrate was concentrated under reduced pressure. The residue was purified by column chromatography (SiO₂, Petroleum ether/Ethyl acetate=3/1 to 0/1) to give title Compound (5.60 g, 14.4 mmol, 55.9% yield) as a white solid. LCMS (ES⁺): m/z 389.1 [M+H]⁺.

Synthesis of trans-tert-butyl (1r,4r)-4-(4-(3-(2,4-dioxotetrahydropyrimidin-1(2H)-yl)-5-fluoro-1-methyl-1H-indazol-6-yl)-3,6-dihydropyridin-1(2H)-yl)cyclohexane-1-carboxylate. To a solution of 1-(5-fluoro-1-methyl-6-(4,4,5,5-tetramethyl-1,3,2-dioxaborolan-2-yl)-1H-indazol-3-yl)dihydropyrimidine-2,4(1H,3H)-dione (986 mg, 2.54 mmol, 1.50 eq) in dioxane (8 mL) was added trans-tert-butyl (1r,4r)-4-(4-(((trifluoromethyl)sulfonyl)oxy)-3,6-dihydropyridin-1(2H)-yl)cyclohexane-1-carboxylate (700 mg, 1.69 mmol, 1.00 eq), K₃PO₄ (1.08 g, 5.08 mmol, 3.00 eq) in H₂O (0.8 mL) and XPhos Pd G3 (143 mg, 169 μmol, 0.100 eq) at 25 °C. The mixture was stirred at 90 °C for 12 h under N₂. LC-MS showed that 1-(5-fluoro-1-methyl-6-(4,4,5,5-tetramethyl-1,3,2-dioxaborolan-2-yl)-1H-indazol-3-yl)dihydropyrimidine-2,4(1H,3H)-dione was consumed completely and the desired mass was detected. The reaction mixture was diluted with water (8 mL) and extracted with EtOAc (8 mL*3). The combined organic layers were washed with brine (8 mL*3), dried over Na₂SO₄, filtered and the filtrate was concentrated under reduced pressure. The residue was purified by column chromatography (SiO₂, Petroleum ether/Ethyl acetate=3/1 to 1/1) to give title Compound (1.50 g, 2.85 mmol, 84.3% yield) as a yellow solid. LCMS (ES⁺): m/z 526.3 [M+H]⁺.

Synthesis of trans-tert-butyl (1r,4r)-4-(4-(3-(2,4-dioxotetrahydropyrimidin-1(2H)-yl)-5-fluoro-1-methyl-1H-indazol-6-yl)piperidin-1-yl)cyclohexane-1-carboxylate. To a solution of 20% Pd(OH)₂/C (100 mg, 50.0% purity) in THF (10 mL) was added trans-tert-butyl (1r,4r)-4-(4-(3-(2,4-dioxotetrahydropyrimidin-1(2H)-yl)-5-fluoro-1-methyl-1H-indazol-6-yl)-3,6-dihydropyridin-1(2H)-yl)cyclohexane-1-carboxylate (500 mg, 951 μmol, 1.00 eq) at 25 °C. The mixture was stirred at 50 °C for 12 h under H₂ (15 psi). LC-MS showed that trans-tert-butyl (1r,4r)-4-(4-(3-(2,4-dioxotetrahydropyrimidin-1(2H)-yl)-5-fluoro-1-methyl-1H-indazol-6-yl)-3,6-dihydropyridin-1(2H)-yl)cyclohexane-1-carboxylate was consumed completely and the desired mass was detected. The reaction mixture was filtered and the filtrate was concentrated under reduced pressure to give title Compound (300 mg, crude) as a yellow solid. LCMS (ES⁺): m/z 528.4 [M+H]⁺.

Synthesis of trans-(1r,4r)-4-(4-(3-(2,4-dioxotetrahydropyrimidin-1(2H)-yl)-5-fluoro-1-methyl-1H-indazol-6-yl)piperidin-1-yl)cyclohexane-1-carboxylic acid. To a solution of trans-tert-butyl (1r,4r)-4-(4-(3-(2,4-dioxotetrahydropyrimidin-1(2H)-yl)-5-fluoro-1-methyl-1H-indazol-6-yl)piperidin-1-yl)cyclohexane-1-carboxylate (250 mg, 474 μmol, 1.00 eq) in DCM (2 mL) was added TFA (1.54 g, 13.5 mmol, 1.00 mL, 28.4 eq) at 25 °C. The mixture was stirred at 25 °C for 1 h. LC-MS showed trans-tert-butyl (1r,4r)-4-(4-(3-(2,4-dioxotetrahydropyrimidin-1(2H)-yl)-5-fluoro-1-methyl-1H-indazol-6-yl)piperidin-1-yl)cyclohexane-1-carboxylate was consumed completely and the desired mass was detected. The mixture was concentrated under reduced pressure to give title Compound (250 mg, crude) as a yellow oil. LCMS (ES⁺): m/z 472.2 [M+H]⁺.

Synthesis of trans-4-(4-(3-(2,4-dioxotetrahydropyrimidin-1(2H)-yl)-5-fluoro-1-methyl-1H-indazol-6-yl)piperidin-1-yl)-N-(trans-4-((5-fluoro-4-(3-(2-oxopyridin-1(2H)-yl)phenyl)pyrimidin-2-yl)amino)cyclohexyl)cyclohexane-1-carboxamide (Compound 512). To a solution of trans-(1r,4r)-4-(4-(3-(2,4-dioxotetrahydropyrimidin-1(2H)-yl)-5-fluoro-1-methyl-1H-indazol-6-yl)piperidin-1-yl)cyclohexane-1-carboxylic acid (250 mg, 530 μmol, 1.00 eq) in DMF (3 mL) was added trans-1-(3-(2-(((1r,4r)-4-aminocyclohexyl)amino)-5-fluoropyrimidin-4-yl)phenyl)pyridin-2(1H)-one (201 mg, 407 μmol, 0.770 eq, TFA), DIEA (206 mg, 1.59 mmol, 277 μL, 3.00 eq) and HATU (302 mg, 795 μmol, 1.50 eq) at 25 °C. The mixture was stirred at 25 °C for 1 h. LC-MS showed trans-(1r,4r)-4-(4-(3-(2,4-dioxotetrahydropyrimidin-1(2H)-yl)-5-fluoro-1-methyl-1H-indazol-6-yl)piperidin-1-yl)cyclohexane-1-carboxylic acid was consumed completely and the desired mass was detected. The reaction mixture was concentrated under reduced pressure. The residue was purified by prep-HPLC (column: Phenomenex luna C18 100*40 mm*3 um; mobile phase: [H₂O (0.2% FA)-ACN]; gradient: 15%-40% B over 8.0 min) to give title Compound (Compound 512) (19.2 mg, 21.6 μmol, 4.09% yield, 99.3% purity, FA) as a white solid. LCMS (ES+): m/z 833.4 [M+H]⁺.

### Example 468. Synthesis of trans-2-(1-(4-((2,6-dioxopiperidin-3-yl)amino)-2-fluorophenyl)piperidin-4-yl)-N-((1r,4r)-4-((5-fluoro-4-(3-(2-oxopyridin-1(2H)-yl)phenyl)pyrimidin-2-yl)amino)cyclohexyl)-2-azaspiro[3.3]heptane-6-carboxamide (Compound 575)

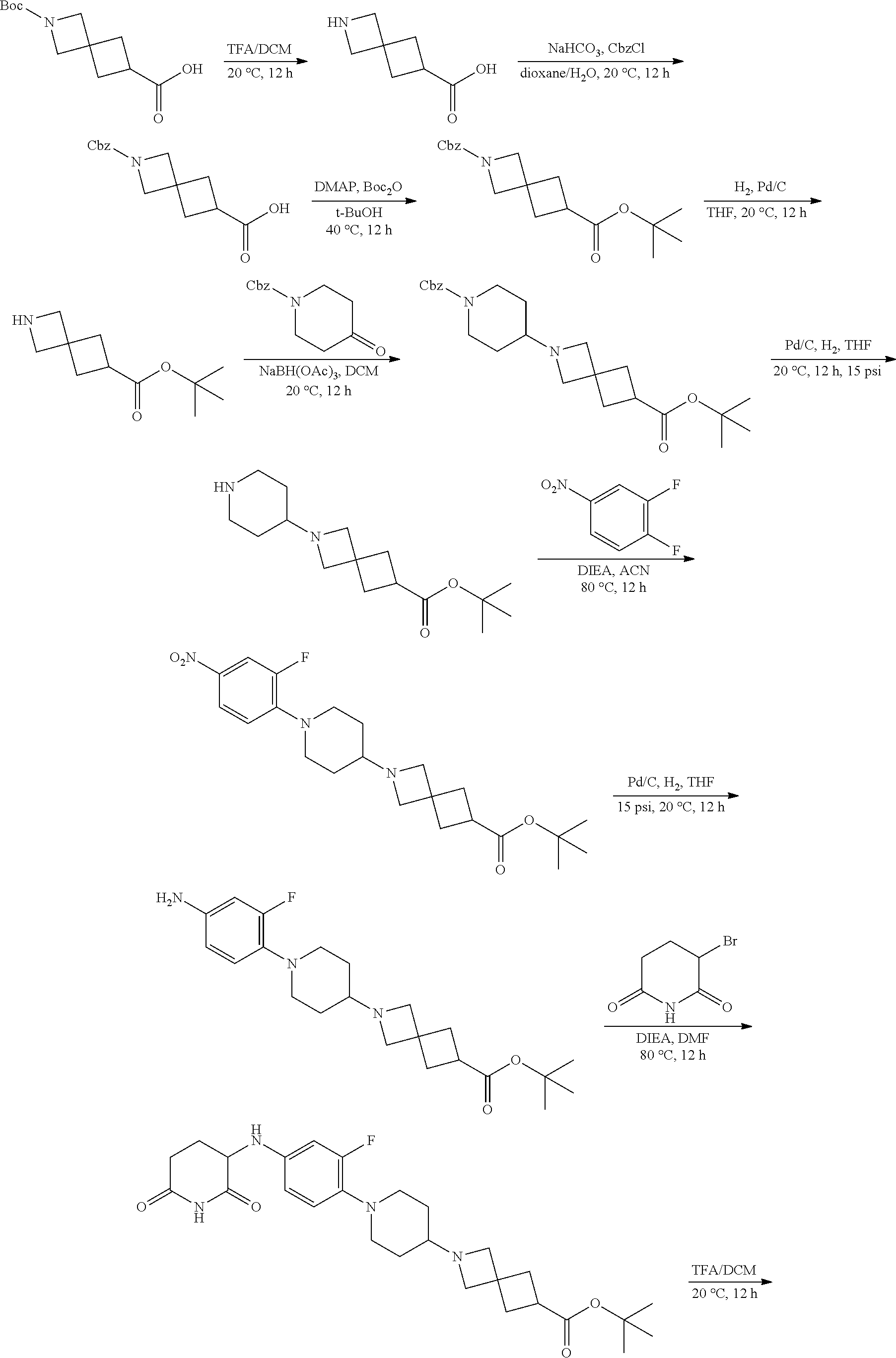

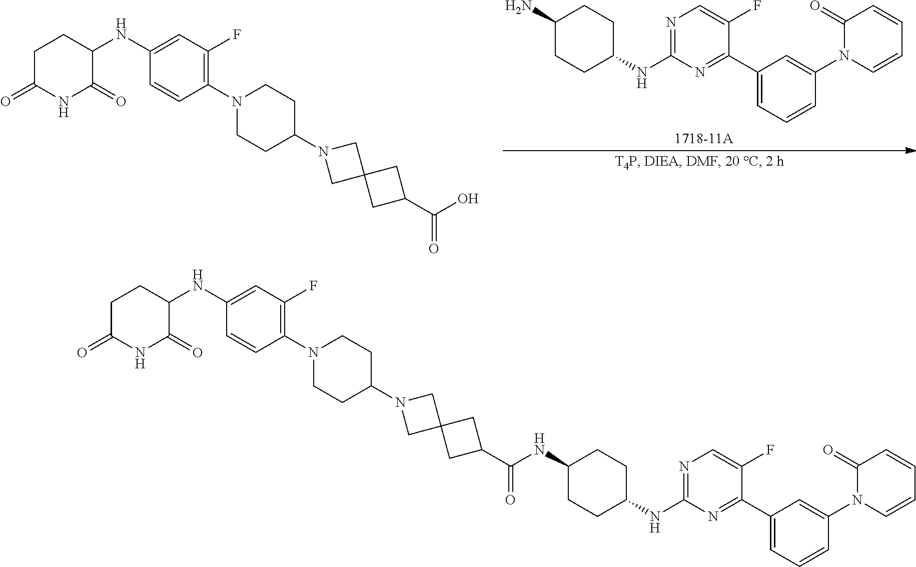 [SMILES: O=C1CCC(Nc2ccc(N3CCC(N4CC5(CC(C(=O)O)C5)C4)CC3)c(F)c2)C(=O)N1 ; N[C@H]1CC[C@H](Nc2ncc(F)c(-c3cccc(-n4ccccc4=O)c3)n2)CC1 ; O=C1CCC(Nc2ccc(N3CCC(N4CC5(CC(C(=O)N[C@H]6CC[C@H](Nc7ncc(F)c(-c8cccc(-n9ccccc9=O)c8)n7)CC6)C5)C4)CC3)c(F)c2)C(=O)N1]

Synthesis of 2-azaspiro[3.3]heptane-6-carboxylic acid. To a solution of 2-(tert-butoxycarbonyl)-2-azaspiro[3.3]heptane-6-carboxylic acid (4.00 g, 16.6 mmol, 1.00 eq) in DCM (15 mL) was added TFA (7.68 g, 67.3 mmol, 5 mL, 4.06 eq) at 20 °C. The mixture was stirred at 20 °C for 12 h. LC-MS showed that 2-(tert-butoxycarbonyl)-2-azaspiro[3.3]heptane-6-carboxylic acid was consumed completely and one main peak with the desired mass was detected. The mixture was concentrated under reduced pressure to give the title compound (4.00 g, crude, TFA) as a yellow oil. LCMS (ES⁺): m/z 142.2 [M+H]⁺.

Synthesis of 2-((benzyloxy)carbonyl)-2-azaspiro[3.3]heptane-6-carboxylic acid. To a solution of 2-azaspiro[3.3]heptane-6-carboxylic acid (3.90 g, 15.3 mmol, 1.00 eq, TFA) in dioxane (40 mL) and H₂O (4 mL) were added NaHCO₃ (1.93 g, 22.9 mmol, 892 μL, 1.50 eq) and CbzCl (3.13 g, 18.3 mmol, 2.62 mL, 1.20 eq) at 0 °C. The mixture was stirred at 20 °C for 12 h. LC-MS showed that 2-azaspiro[3.3]heptane-6-carboxylic acid was consumed completely and one main peak with the desired mass was detected. The mixture was concentrated under reduced pressure. The reaction mixture was quenched by addition H₂O (20 ml), 1M HCl was added to the mixture to adjust pH~8, and extracted with EtOAc (30 mL*3), dried over Na₂SO₄, filtered and concentrated under reduced pressure. The crude product was triturated with EtOAc (10 ml), filtered and the filter cake was concentrated under reduced pressure to give the title compound (2.20 g, 7.99 mmol, 52.3% yield, N/A purity) as a white solid. LCMS (ES⁺): m/z 276.1 [M+H]⁺.

Synthesis of 2-benzyl 6-(tert-butyl) 2-azaspiro[3.3]heptane-2,6-dicarboxylate. To a solution of 2-((benzyloxy)carbonyl)-2-azaspiro[3.3]heptane-6-carboxylic acid (2.70 g, 9.81 mmol, 1.00 eq) in t-BuOH (20 mL) were added Boc₂O (2.57 g, 11.8 mmol, 2.70 mL, 1.20 eq) and DMAP (119 mg, 974 μmol, 9.93e-2 eq) at 20 °C. The mixture was stirred at 40 °C for 12 h. LC-MS showed that 2-((benzyloxy)carbonyl)-2-azaspiro[3.3]heptane-6-carboxylic acid was consumed completely and one main peak with the desired mass was detected. The mixture was concentrated under reduced pressure. The residue was purified by column chromatography (SiO₂, Petroleum ether/Ethyl acetate=1:0 to 3:1) to give the title compound (3.40 g, crude) as a light yellow oil. LCMS (ES⁺): m/z 332.2 [M+H]⁺.

Synthesis of tert-butyl 2-azaspiro[3.3]heptane-6-carboxylate. To a mixture of 10% Pd/C (3.00 g, 50% purity) in THF (50 mL) was added 2-benzyl 6-(tert-butyl) 2-azaspiro[3.3]heptane-2,6-dicarboxylate (3.40 g, 10.3 mmol, 1.00 eq). The mixture was stirred at 20 °C for 12 h under H₂ (15 Psi). LC-MS showed that 2-benzyl 6-(tert-butyl) 2-azaspiro[3.3]heptane-2,6-dicarboxylate was consumed completely and trace desired mass was detected. The mixture was filtered and the filtrate was concentrated under reduced pressure to give the title compound (2.00 g, crude) as a colorless oil. LCMS (ES⁺): m/z 198.1 [M+H]⁺.

Synthesis of tert-butyl 2-(1-((benzyloxy)carbonyl)piperidin-4-yl)-2-azaspiro[3.3]heptane-6-carboxylate. To a solution of benzyl 4-oxopiperidine-1-carboxylate (2.30 g, 9.86 mmol, 1.96 mL, 1.00 eq) in DCM (20 mL) were added tert-butyl 2-azaspiro[3.3]heptane-6-carboxylate (1.95 g, 9.86 mmol, 1.00 eq) and NaBH(OAc)₃ (6.27 g, 29.6 mmol, 3.00 eq) at 20 °C. The mixture was stirred at 20 °C for 12 h. LC-MS showed that tert-butyl 2-azaspiro[3.3]heptane-6-carboxylate was consumed completely and one main peak with the desired mass was detected. The mixture was concentrated under reduced pressure. The residue was purified by column chromatography (SiO₂, Petroleum ether; Ethyl acetate=1:0 to 0:1) to give the title compound (3.00 g, 7.24 mmol, 73.4% yield, n/a purity) as a yellow oil. LCMS (ES⁺): m/z 415.2 [M+H]⁺.

Synthesis of tert-butyl 2-(piperidin-4-yl)-2-azaspiro[3.3]heptane-6-carboxylate. To a mixture of 10% Pd/C (3.00 g, 50% purity) in THF (30 mL) was added tert-butyl 2-(1-((benzyloxy)carbonyl)piperidin-4-yl)-2-azaspiro[3.3]heptane-6-carboxylate (3.00 g, 7.24 mmol, 1.00 eq) at 20 °C. The mixture was stirred at 20 °C for 12 h under H₂ (15 Psi). LC-MS showed that tert-butyl 2-(1-((benzyloxy)carbonyl)piperidin-4-yl)-2-azaspiro[3.3]heptane-6-carboxylate was consumed completely and one main peak with the desired mass was detected. The mixture was filtered and the filtrated was concentrated under reduced pressure to give the title compound (2.00 g, crude) as a light yellow oil. LCMS (ES⁺): m/z 281.2 [M+H]⁺.

Synthesis of tert-butyl 2-(1-(2-fluoro-4-nitrophenyl)piperidin-4-yl)-2-azaspiro[3.3]heptane-6-carboxylate. To a solution of tert-butyl 2-(piperidin-4-yl)-2-azaspiro[3.3]heptane-6-carboxylate (2.00 g, 7.13 mmol, 1.00 eq) in ACN (20 mL) was added 1,2-difluoro-4-nitrobenzene (1.36 g, 8.56 mmol, 948 μL, 1.20 eq) and DIEA (1.84 g, 14.3 mmol, 2.48 mL, 2.00 eq) at 20 °C. The mixture was stirred at 80 °C for 12 h. LC-MS showed that tert-butyl 2-(piperidin-4-yl)-2-azaspiro[3.3]heptane-6-carboxylate was consumed completely and one main peak with the desired mass was detected. The mixture was concentrated under reduced pressure. The residue was purified by column chromatography (SiO₂, Petroleum ether/Ethyl acetate=1/1 to 0/1) to give the title compound (2.50 g, 5.96 mmol, 83.6% yield, n/a purity) as a yellow solid. LCMS (ES⁺): m/z 420.2 [M+H]⁺.

Synthesis of tert-butyl 2-(1-(4-amino-2-fluorophenyl)piperidin-4-yl)-2-azaspiro[3.3]heptane-6-carboxylate. To a mixture of 10% Pd/C (4.00 g, 50% purity) in THE (25 mL) was added tert-butyl 2-(1-(2-fluoro-4-nitrophenyl)piperidin-4-yl)-2-azaspiro[3.3]heptane-6-carboxylate (2.50 g, 5.96 mmol, 1.00 eq) at 20 °C. The mixture was stirred at 20 °C for 12 h under H₂ (15 Psi). LC-MS showed that tert-butyl 2-(1-(2-fluoro-4-nitrophenyl)piperidin-4-yl)-2-azaspiro[3.3]heptane-6-carboxylate was consumed completely and one main peak with the desired mass was detected. The mixture was filtered and the filtrate was concentrated under reduced pressure to give the title compound (2.10 g, crude) as a brown oil. LCMS (ES⁺): m/z 390.2 [M+H]⁺.

Synthesis of tert-butyl 2-(1-(4-((2,6-dioxopiperidin-3-yl)amino)-2-fluorophenyl)piperidin-4-yl)-2-azaspiro[3.3]heptane-6-carboxylate. To a solution of tert-butyl 2-(1-(4-amino-2-fluorophenyl)piperidin-4-yl)-2-azaspiro[3.3]heptane-6-carboxylate (2.00 g, 5.13 mmol, 1.00 eq) in DMF (20 mL) were added 3-bromopiperidine-2,6-dione (3.94 g, 20.5 mmol, 4.00 eq) and DIEA (1.33 g, 10.3 mmol, 1.79 mL, 2.00 eq) at 20 °C. The mixture was stirred at 80 °C for 12 h. LC-MS showed that tert-butyl 2-(1-(4-amino-2-fluorophenyl)piperidin-4-yl)-2-azaspiro[3.3]heptane-6-carboxylate was consumed completely and one peak with the desired mass was detected. The reaction was concentrated under reduced pressure. The residue was purified by column chromatography (SiO₂, Petroleum ether/Ethyl acetate=1/0 to 0/1) to give the title compound (2.00 g, 4.00 mmol, 77.8% yield, n/a purity) as a green solid. LCMS (ES⁺): m/z 501.1 [M+H]⁺.

Synthesis of 2-(1-(4-((2,6-dioxopiperidin-3-yl)amino)-2-fluorophenyl)piperidin-4-yl)-2-azaspiro[3.3]heptane-6-carboxylic acid. To a solution of tert-butyl 2-(1-(4-((2,6-dioxopiperidin-3-yl)amino)-2-fluorophenyl)piperidin-4-yl)-2-azaspiro[3.3]heptane-6-carboxylate (600 mg, 1.20 mmol, 1.00 eq) in DCM (6 mL) was added TFA (3.07 g, 26.9 mmol, 2 mL, 22.5 eq) at 20 °C. The mixture was stirred at 20 °C for 12 h. LC-MS showed that tert-butyl 2-(1-(4-((2,6-dioxopiperidin-3-yl)amino)-2-fluorophenyl)piperidin-4-yl)-2-azaspiro[3.3]heptane-6-carboxylate was consumed completely and one main peak with the desired mass was detected. The mixture was concentrated under reduced pressure to give he title compound (600 mg, crude, TFA) as a green oil LCMS (ES⁺): m/z 445.2 [M+H]⁺.

Synthesis of trans-2-(1-(4-((2,6-dioxopiperidin-3-yl)amino)-2-fluorophenyl)piperidin-4-yl)-N-((1r,4r)-4-((5-fluoro-4-(3-(2-oxopyridin-1(2H)-yl)phenyl)pyrimidin-2-yl)amino)cyclohexyl)-2-azaspiro[3.3]heptane-6-carboxamide (Compound 575). To a solution of trans-2-(1-(4-((2,6-dioxopiperidin-3-yl)amino)-2-fluorophenyl)piperidin-4-yl)-2-azaspiro[3.3]heptane-6-carboxylic acid (600 mg, 1.35 mmol, 1.00 eq) in EtOAc (10 mL) were added trans-1-(3-(2-(((1r,4r)-4-aminocyclohexyl)amino)-5-fluoropyrimidin-4-yl)phenyl)pyridin-2(1H)-one (512 mg, 1.04 mmol, 7.69e-1 eq, TFA), DIEA (523 mg, 4.05 mmol, 705 μL, 3.00 eq) and T₄P (1.95 g, 2.70 mmol, 50% purity, 2.00 eq) at 20 °C. The mixture was stirred at 20 °C for 2 h. LC-MS showed that 2-(1-(4-((2,6-dioxopiperidin-3-yl)amino)-2-fluorophenyl)piperidin-4-yl)-2-azaspiro[3.3]heptane-6-carboxylic acid was consumed completely and one peak with the desired mass was detected. The mixture was concentrated under reduced pressure. The residue was purified by prep-HPLC (column: Phenomenex luna C18 100*40 mm*3 um; mobile phase: [H₂O (0.2% FA)-ACN]; gradient: 10%-40% B over 8.0 min) to give the title compound (Compound 575) (51.1 mg, 62.6 μmol, 4.64% yield, 98.7% purity, 0.8 FA) as a pale yellow solid. LCMS (ES⁺): m/z 806.3 [M+H]⁺.

### Example 469. Synthesis of trans-2-((1r,4R)-4-(4-(3-(2,6-dioxopiperidin-3-yl)-1-methyl-1H-indazol-6-yl)piperidin-1-yl)cyclohexyl)-N-((1r,4R)-4-((5-fluoro-4-(3-(2-oxopyridin-1(2H)-yl)phenyl)pyrimidin-2-yl)amino)cyclohexyl)acetamide (Compound 507)

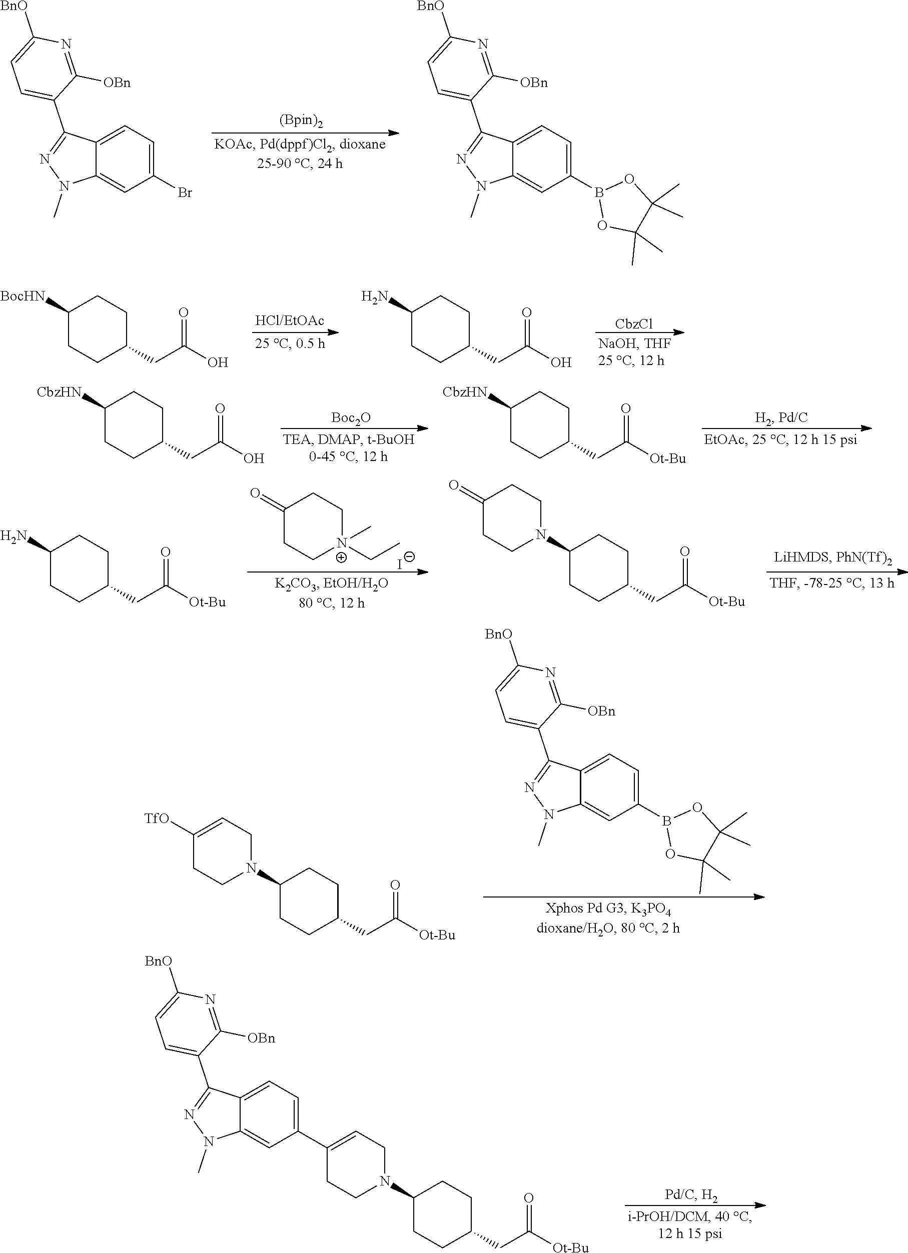

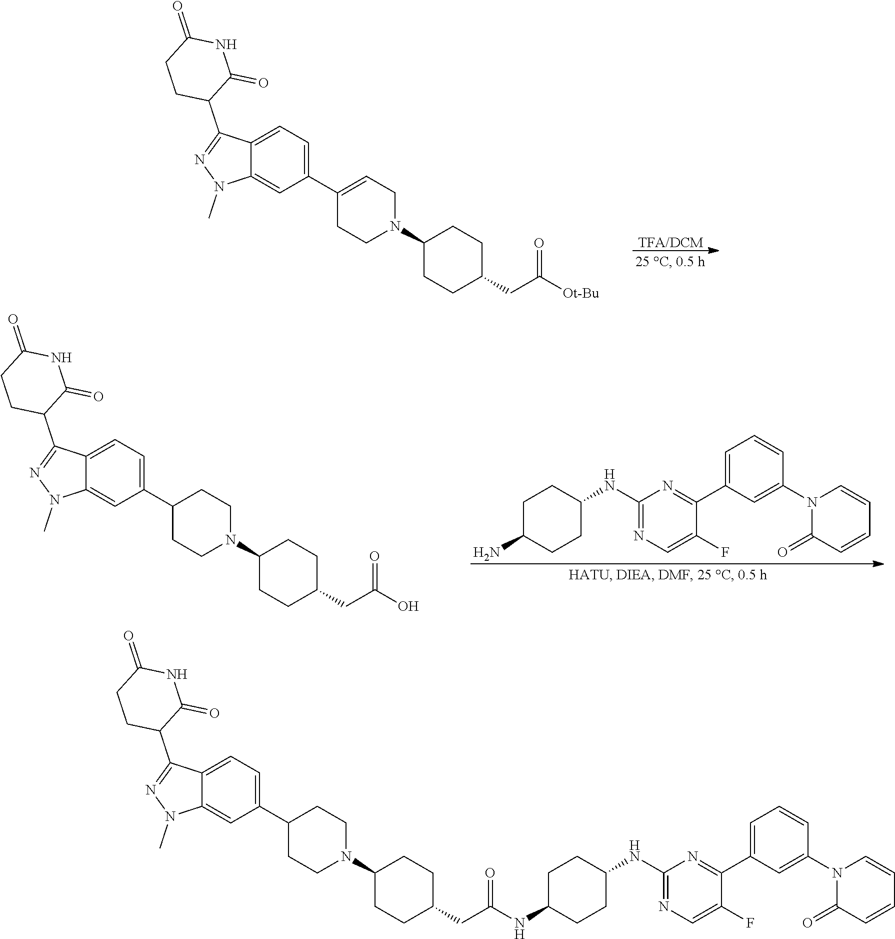 [SMILES: Cn1nc(C2CCC(=O)NC2=O)c2ccc(C3=CCN([C@H]4CC[C@H](CC(=O)OC(C)(C)C)CC4)CC3)cc21 ; "TFA/DCM" ; Cn1nc(C2CCC(=O)NC2=O)c2ccc(C3CCN([C@H]4CC[C@H](CC(=O)O)CC4)CC3)cc21 ; N[C@H]1CC[C@H](Nc2ncc(F)c(-c3cccc(-n4ccccc4=O)c3)n2)CC1 ; Cn1nc(C2CCC(=O)NC2=O)c2ccc(C3CCN([C@H]4CC[C@H](CC(=O)N[C@H]5CC[C@H](Nc6ncc(F)c(-c7cccc(-n8ccccc8=O)c7)n6)CC5)CC4)CC3)cc21]

Synthesis of 3-(2,6-bis(benzyloxy)pyridin-3-yl)-1-methyl-6-(4,4,5,5-tetramethyl-1,3,2-dioxaborolan-2-yl)-1H-indazole. To a solution of 3-(2,6-bis(benzyloxy)pyridin-3-yl)-6-bromo-1-methyl-1H-indazole (5.00 g, 9.99 mmol, 1.00 eq) in dioxane (100 mL) were added 4,4,4′,4′,5,5,5′,5′-octamethyl-2,2′-bi(1,3,2-dioxaborolane) (3.04 g, 12.0 mmol, 1.20 eq), KOAc (1.96 g, 20.0 mmol, 2.00 eq) and Pd(dppf)Cl₂ (366 mg, 500 μmol, 0.0500 eq) at 25 °C under N₂ atmosphere. The mixture was stirred at 90 °C for 12 h under N₂ atmosphere. LC-MS showed 3-(2,6-bis(benzyloxy)pyridin-3-yl)-6-bromo-1-methyl-1H-indazole was remaining and the desired mass was detected. Then 4,4,4′,4′,5,5,5′,5′-octamethyl-2,2′-bi(1,3,2-dioxaborolane) (3.04 g, 12.0 mmol, 1.20 eq), Pd(dppf)Cl₂ (366 mg, 500 μmol, 0.0500 eq) and KOAc (1.96 g, 20.0 mmol, 2.00 eq) were added to the mixture at 25 °C under N₂ atmosphere. The mixture was stirred at 90 °C for another 12 h under N₂ atmosphere. LC-MS showed 3-(2,6-bis(benzyloxy)pyridin-3-yl)-6-bromo-1-methyl-1H-indazole was consumed completely and the desired mass was detected. The reaction mixture was filtered and the filtrate was concentrated under reduced pressure to give a residue. To the residue was added H₂O (60 mL) and the mixture was extracted with EtOAc (60 mL*3). The combined organic layers were washed with brine (60 mL), dried over Na₂SO₄, filtered and the filtrate was concentrated under reduced pressure to give a residue. The residue was purified by column chromatography (SiO₂, Petroleum ether:Ethyl acetate=10:1 to 5:1) to give the title compound (5.00 g, 9.13 mmol, 91.4% yield) as a brown oil. LCMS (ES⁺): m/z 548.3 [M+H]⁺.

Synthesis trans-2-(4-aminocyclohexyl)acetic acid. To a solution of trans-2-(4-((tert-butoxycarbonyl)amino)cyclohexyl)acetic acid (47.0 g, 183 mmol, 1.00 eq) in HCl/EtOAc (500 mL) was stirred at 25 °C for 0.5 h. LC-MS showed trans-2-(4-((tert-butoxycarbonyl)amino)cyclohexyl)acetic acid was consumed completely and the desired mass was detected. The reaction mixture was concentrated under reduced pressure to give the title compound (35.4 g, crude, HCl) as a white solid. LCMS (ES⁺): m/z 158.2 [M+H]⁺.

Synthesis of trans-2-(4-(((benzyloxy)carbonyl)amino)cyclohexyl)acetic acid. To a solution of trans-2-(4-aminocyclohexyl)acetic acid (35.4 g, 183 mmol, 1.00 eq, HCl) in THF (500 mL) was added NaOH (2 M, 183 mL, 2.00 eq, in H₂O) at 25 °C. Then CbzCl (34.3 g, 201 mmol, 28.7 mL, 1.10 eq) was added to the mixture at 0 °C. The mixture was stirred at 25 °C for 12 h. LC-MS showed trans-2-(4-aminocyclohexyl)acetic acid was consumed completely and the desired mass was detected. The mixture was concentrated under reduced pressure to remove THF. Then the remaining aqueous layer was acidified by HCl (1 M) to adjust pH to 4. The mixture was filtered and the filter cake was dried under reduced pressure to give the title compound (43.0 g, crude) as a white solid. LCMS (ES⁺): m/z 292.3 [M+H]⁺.

Synthesis of trans-tert-butyl 2-(4-(((benzyloxy)carbonyl)amino)cyclohexyl)acetate. To a solution of trans-2-(4-(((benzyloxy)carbonyl)amino)cyclohexyl)acetic acid (39.0 g, 134 mmol, 1.00 eq) in t-BuOH (3000 mL) were added TEA (61.0 g, 602 mmol, 83.8 mL, 4.50 eq) at 25 °C. Then DMAP (19.6 g, 161 mmol, 1.20 eq) and Boc₂O (292 g, 1.34 μmol, 308 mL, 10.0 eq) were added to the mixture at 0 °C. The mixture was stirred at 45 °C for 12 h. LC-MS showed trans-2-(4-(((benzyloxy)carbonyl)amino)cyclohexyl)acetic acid was consumed completely and the desired mass was detected. The reaction mixture was concentrated under reduced pressure to remove solvent, then H₂O (2000 mL) was added to the mixture at 25 °C. The reaction mixture was extracted with EtOAc (1000 mL*3). The organic layers were dried over anhydrous sodium sulfate, filtered and the filtrate was concentrated under reduced pressure to give a residue. The residue was purified by MPLC (SiO₂, Petroleum ether:Ethyl acetate=1:0 to 3:1) to give the title compound (26.0 g, 74.8 mmol, 55.9% yield) as a white solid. LCMS (ES⁺): m/z 292.3 [M+H−56]⁺.

Synthesis of trans-tert-butyl 2-(4-aminocyclohexyl)acetate. 10% Pd/C (10.0 g, 57.6 mmol, 50.0% purity, 1.00 eq) was added to EtOAc (300 mL) at 25 °C under N₂ atmosphere, then trans-tert-butyl 2-(4-(((benzyloxy)carbonyl)amino)cyclohexyl)acetate (20.0 g, 57.6 mmol, 1.00 eq) was added to the mixture at 25 °C, the suspension was degassed and purged with H₂ for three times. The mixture was stirred at 25 °C for 12 h under H₂ (15 psi) atmosphere. LC-MS showed trans-tert-butyl 2-(4-(((benzyloxy)carbonyl)amino)cyclohexyl)acetate was consumed completely and the desired mass was detected. The reaction mixture was filtered and the filtrate was concentrated under reduced pressure to give the title compound (12.3 g, crude) as a brown oil. LCMS (ES⁺): m/z 214.3 [M+H]⁺.

Synthesis of trans-tert-butyl 2-(4-(4-oxopiperidin-1-yl)cyclohexyl)acetate. To a solution of trans-tert-butyl 2-(4-aminocyclohexyl)acetate (8.20 g, 38.4 mmol, 1.00 eq) in EtOH (96 mL) and H₂O (60 mL) were added K₂CO₃ (6.38 g, 46.1 mmol, 1.20 eq) and 1-ethyl-1-methyl-4-oxopiperidin-1-ium iodide (6.56 g, 46.1 mmol, 1.20 eq) at 25 °C. The mixture was stirred at 80 °C for 12 h. LC-MS showed trans-tert-butyl 2-(4-aminocyclohexyl)acetate was consumed completely and the desired mass was detected. The reaction mixture was concentrated under reduced pressure to remove EtOH. Then the remaining aqueous phase was extracted with EtOAc (50 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=5:1 to 3:1) to give the title compound (6.00 g, 20.3 mmol, 52.8% yield) as a white solid. LCMS (ES⁺): m/z 296.3 [M+H]⁺.

Synthesis of trans-tert-butyl 2-(4-(4-(((trifluoromethyl)sulfonyl)oxy)-3,6-dihydropyridin-1(2H)-yl)cyclohexyl)acetate. To a solution of trans-tert-butyl 2-(4-(4-oxopiperidin-1-yl)cyclohexyl)acetate (5.90 g, 20.0 mmol, 1.00 eq) in THF (200 mL) was added LiHMDS (1 M solution in THF, 39.9 mL, 6.68 g, 2.00 eq) at −78 °C under N₂ atmosphere. The mixture was stirred at −78 °C for 1 h under N₂ atmosphere. Then PhN(Tf)₂ (10.7 g, 30.0 mmol, 1.50 eq) in THF (50 mL) was added to the mixture at −78 °C under N₂ atmosphere. The mixture was slowly warmed to 25 °C and stirred at 25 °C for 12 h under N₂ atmosphere. LC-MS showed the desired mass was detected. Saturated NH₄Cl aqueous solution (200 mL) was added to the mixture dropwise to quench the reaction at 0 °C under N₂ atmosphere. Then the reaction mixture was extracted with EtOAc (100 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=10:1 to 5:1) to give the title compound (1.80 g, 4.21 mmol, 21.1% yield) as a white solid. LCMS (ES⁺): m/z 428.2 [M+H]⁺.

Synthesis of trans-tert-butyl 2-(4-(4-(3-(2,6-bis(benzyloxy)pyridin-3-yl)-1-methyl-1H-indazol-6-yl)-3,6-dihydropyridin-1(2H)-yl)cyclohexyl)acetate. To a solution of trans-tert-butyl 2-(4-(4-(((trifluoromethyl)sulfonyl)oxy)-3,6-dihydropyridin-1(2H)-yl)cyclohexyl)acetate (1.50 g, 3.51 mmol, 1.00 eq) in dioxane (40 mL) and H₂O (20 mL) were added 3-(2,6-bis(benzyloxy)pyridin-3-yl)-1-methyl-6-(4,4,5,5-tetramethyl-1,3,2-dioxaborolan-2-yl)-1H-indazole (1.54 g, 2.81 mmol, 0.800 eq), K₃PO₄ (2.23 g, 10.5 mmol, 3.00 eq) and XPhos Pd G3 (297 mg, 351 μmol, 0.100 eq) at 25 °C under N₂ atmosphere. The mixture was stirred at 80 °C for 2 h under N₂ atmosphere. LC-MS showed trans-tert-butyl 2-(4-(4-(((trifluoromethyl)sulfonyl)oxy)-3,6-dihydropyridin-1(2H)-yl)cyclohexyl)acetate was consumed completely and the desired mass was detected. Water (30 mL) was added to the mixture at 25 °C. Then the reaction mixture was extracted with EtOAc (50 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=5:1 to 3:1) to give the title compound (300 mg, 430 μmol, 12.2% yield) as a white solid. LCMS (ES⁺): m/z 699.4 [M+H]⁺.

Synthesis of trans-tert-butyl 2-(4-(4-(3-(2,6-dioxopiperidin-3-yl)-1-methyl-1H-indazol-6-yl)piperidin-1-yl)cyclohexyl)acetate. 10% Pd/C (300 mg, 50% purity) was added to the reaction flask at 25 °C under N₂ atmosphere, and then i-PrOH (5 mL) and DCM (5 mL) were added to the reaction flask, then trans-tert-butyl 2-(4-(4-(3-(2,6-bis(benzyloxy)pyridin-3-yl)-1-methyl-1H-indazol-6-yl)-3,6-dihydropyridin-1(2H)-yl)cyclohexyl)acetate (300 mg, 429 μmol, 1.00 eq) was added to the mixture at 25 °C, the suspension was degassed and purged with H₂ for three times. The mixture was stirred at 40 °C for 12 h under H₂ (15 psi) atmosphere. LC-MS showed trans-tert-butyl 2-(4-(4-(3-(2,6-bis(benzyloxy)pyridin-3-yl)-1-methyl-1H-indazol-6-yl)-3,6-dihydropyridin-1(2H)-yl)cyclohexyl)acetate was consumed completely and the desired mass was detected. The reaction mixture was filtered and the filtrate was concentrated under reduced pressure to give the title compound (190 mg, crude) as a brown oil. LCMS (ES⁺): m/z 523.3 [M+H]⁺.

Synthesis of trans-2-(4-(4-(3-(2,6-dioxopiperidin-3-yl)-1-methyl-1H-indazol-6-yl)piperidin-1-yl)cyclohexyl)acetic acid. To a solution of trans-tert-butyl 2-(4-(4-(3-(2,6-dioxopiperidin-3-yl)-1-methyl-1H-indazol-6-yl)piperidin-1-yl)cyclohexyl)acetate (180 mg, 344 μmol, 1.00 eq) in DCM (3 mL) was added TFA (1 mL) at 25 °C. The mixture was stirred at 25 °C for 0.5 h. LC-MS showed trans-tert-butyl 2-(4-(4-(3-(2,6-dioxopiperidin-3-yl)-1-methyl-1H-indazol-6-yl)piperidin-1-yl)cyclohexyl)acetate was consumed completely and the desired mass was detected. The reaction mixture was concentrated under reduced pressure to give the title compound (160 mg, crude) as a yellow oil. LCMS (ES⁺): m/z 467.3 [M+H]⁺.

Synthesis of trans-2-((1r,4R)-4-(4-(3-(2,6-dioxopiperidin-3-yl)-1-methyl-1H-indazol-6-yl)piperidin-1-yl)cyclohexyl)-N-((1r,4R)-4-((5-fluoro-4-(3-(2-oxopyridin-1(2H)-yl)phenyl)pyrimidin-2-yl)amino)cyclohexyl)acetamide (Compound 507). To a solution of trans-2-(4-(4-(3-(2,6-dioxopiperidin-3-yl)-1-methyl-1H-indazol-6-yl)piperidin-1-yl)cyclohexyl)acetic acid (160 mg, 343 μmol, 1.00 eq) in DMF (5 mL) were added HATU (52.2 mg, 137 μmol, 0.400 eq), DIEA (133 mg, 1.03 mmol, 179 μL, 3.00 eq) and trans-1-(3-(2-((4-aminocyclohexyl)amino)-5-fluoropyrimidin-4-yl)phenyl)pyridin-2(1H)-one (118 mg, 240 μmol, 0.700 eq, TFA) at 25 °C. The mixture was stirred at 25 °C for 0.5 h. LC-MS showed trans-2-(4-(4-(3-(2,6-dioxopiperidin-3-yl)-1-methyl-1H-indazol-6-yl)piperidin-1-yl)cyclohexyl)acetic acid was consumed completely, and the desired mass was detected. The reaction mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-80% B over 8.0 min) to give the title compound (Compound 507) (36.2 mg, 39.5 μmol, 11.5% yield, 90.3% purity) as a off-white solid. LCMS (ES⁺): m/z 828.5 [M+H]⁺

### Example 470. Synthesis of trans-(1r,4R)-4-((4-(3-(2,6-dioxopiperidin-3-yl)-1-methyl-1H-indazol-6-yl)piperazin-1-yl)methyl)-N-((1r,4R)-4-((5-fluoro-4-(3-(2-oxopyridin-1(2H)-yl)phenyl)pyrimidin-2-yl)amino)cyclohexyl)cyclohexane-1-carboxamide (Compound 489)

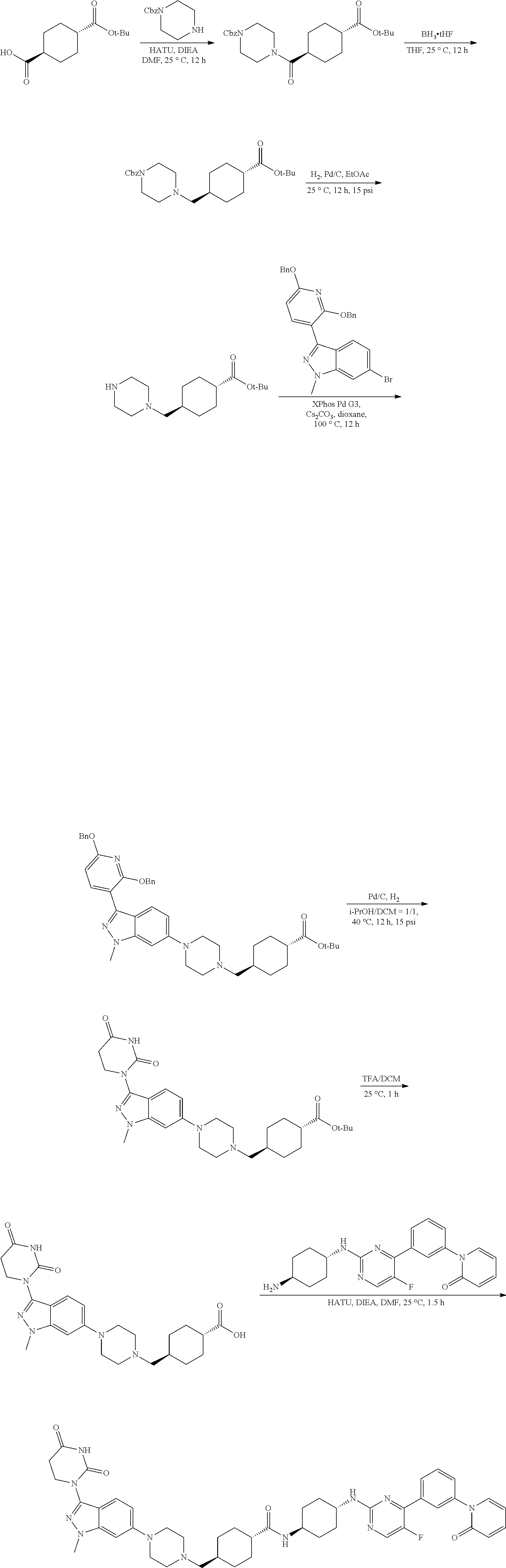

Synthesis of trans-benzyl 4-(4-(tert-butoxycarbonyl)cyclohexane-1-carbonyl)piperazine-1-carboxylate. To a solution of trans-4-(tert-butoxycarbonyl)cyclohexane-1-carboxylic acid (20.0 g, 87.6 mmol, 1.00 eq) in DMF (200 mL) were added HATU (40.0 g, 105 mmol, 1.20 eq), DIEA (22.7 g, 175 mmol, 30.5 mL, 2.00 eq) and benzyl piperazine-1-carboxylate (19.3 g, 87.6 mmol, 16.9 mL, 1.00 eq) at 25 °C. The mixture was stirred at 25 °C for 12 h. LCMS showed trans-4-(tert-butoxycarbonyl)cyclohexane-1-carboxylic acid was consumed completely and the desired mass was detected. TLC (SiO₂, Petroleum ether:Ethyl acetate=1:1) showed trans-4-(tert-butoxycarbonyl)cyclohexane-1-carboxylic acid was consumed completely, and three new spots formed. Water (200 mL) was added to the mixture at 25 °C. Then the reaction mixture was extracted with EtOAc (150 mL*4). The organic layers were dried over anhydrous sodium sulfate, filtered and the filtrate was concentrated under reduced pressure to give a residue. The residue was purified by column chromatography (SiO₂, Petroleum ether:Ethyl acetate=2:1 to 1:1) to give the title compound (13.0 g, 30.2 mmol, 34.5% yield) as a white solid. LCMS (ES⁺): m/z 431.3 [M+H]⁺.

Synthesis of trans-benzyl 4-((4-(tert-butoxycarbonyl)cyclohexyl)methyl)piperazine-1-carboxylate. To a solution of trans-benzyl 4-(4-(tert-butoxycarbonyl)cyclohexane-1-carbonyl)piperazine-1-carboxylate (4.00 g, 9.29 mmol, 1.00 eq) in THE (80 mL) was added BH₃·THF (1.00 M in THF, 23.2 mL, 2.50 eq) under N₂ atmosphere at 0 °C. The mixture was stirred at 25 °C for 12 h. LCMS showed trans-benzyl 4-(4-(tert-butoxycarbonyl)cyclohexane-1-carbonyl)piperazine-1-carboxylate was consumed and the desired mass was detected. MeOH (30 mL) was added to the mixture to quench BH₃·THF at 0 °C. Then the mixture was stirred at 60 °C for 2 h. It was concentrated under reduced pressure to give a residue. The residue was purified by column chromatography (SiO₂, Petroleum ether:Ethyl acetate=10:1 to 4:1) to give the title compound (2.60 g, 5.95 mmol, 64.1% yield, 95.4% purity) as a white solid. LCMS (ES⁺): m/z 417.3 [M+H]⁺.

Synthesis of trans-tert-butyl-4-(piperazin-1-ylmethyl)cyclohexane-1-carboxylate. 10% Pd/C (6.00 g, 50.0% purity) was added to the reaction flask at 25 °C under N₂ atmosphere, and then EtOAc (100 mL) was added to the reaction flask, then trans-benzyl 4-((4-(tert-butoxycarbonyl)cyclohexyl)methyl)piperazine-1-carboxylate (2.60 g, 6.24 mmol, 1.00 eq) was added to the mixture at 25 °C, the suspension was degassed and purged with H₂ for three times. The mixture was stirred at 25 °C for 12 h under H₂ (15 psi) atmosphere. LCMS showed trans-benzyl 4-((4-(tert-butoxycarbonyl)cyclohexyl)methyl)piperazine-1-carboxylate was consume completely and the desired mass was detected. The mixture was filtered and the filtrate was concentrated under reduced pressure to give the title compound (2.40 g, crude) as a yellow oil. LCMS (ES⁺): m/z 283.3 [M+H]⁺.

Synthesis of trans-tert-butyl-4-((4-(3-(2,6-bis(benzyloxy)pyridin-3-yl)-1-methyl-1H-indazol-6-yl)piperazin-1-yl)methyl)cyclohexane-1-carboxylate. To a solution of trans-tert-butyl-4-(piperazin-1-ylmethyl)cyclohexane-1-carboxylate (2.10 g, 7.44 mmol, 1.00 eq) and 6-bromo-3-(2,6-dibenzyloxy-3-pyridyl)-1-methyl-1H-indazole (1.86 g, 3.72 mmol, 0.500 eq) in dioxane (40 mL) were added Cs₂CO₃ (4.85 g, 14.9 mmol, 2.00 eq) and XPhos Pd G3 (629 mg, 744 μmol, 0.100 eq) under N₂ atmosphere at 25 °C. The mixture was stirred at 100 °C for 12 h. LCMS showed trans-tert-butyl-4-(piperazin-1-ylmethyl)cyclohexane-1-carboxylate was consumed completely and the desired mass was detected. The reaction mixture was concentrated under reduced pressure to give a residue. Water (60 mL) was added to the residue at 25 °C. Then the reaction mixture was extracted with EtOAc (60 mL*4). The organic layers were dried over anhydrous sodium sulfate, filtered and the filtrate was concentrated under reduced pressure to give a residue. The residue was purified by column chromatography (SiO₂, Petroleum ether:Ethyl acetate=2:1 to 1:1) to give the title compound (1.60 g, 2.05 mmol, 27.6% yield, 90.1% purity) as a bright yellow oil. LCMS (ES⁺): m/z 702.4 [M+H]⁺.

Synthesis of trans-tert-butyl-4-((4-(3-(2,6-dioxopiperidin-3-yl)-1-methyl-1H-indazol-6-yl)piperazin-1-yl)methyl)cyclohexane-1-carboxylate. 10% Pd/C (0.600 g, 50% purity) was added to the reaction flask at 25 °C under N₂ atmosphere, then i-PrOH (5.00 mL) and DCM (5.00 mL) were added to the reaction flask, then trans-tert-butyl-4-((4-(3-(2,6-bis(benzyloxy)pyridin-3-yl)-1-methyl-1H-indazol-6-yl)piperazin-1-yl)methyl)cyclohexane-1-carboxylate (0.600 g, 855 μmol, 1.00 eq) was added to the mixture at 25 °C, the suspension was degassed and purged with H₂ for three times. The mixture was stirred at 40 °C for 12 h under H₂ (15 psi) atmosphere. LCMS showed trans-tert-butyl-4-((4-(3-(2,6-bis(benzyloxy)pyridin-3-yl)-1-methyl-1H-indazol-6-yl)piperazin-1-yl)methyl)cyclohexane-1-carboxylate was consumed completely and the desired mass was detected. The mixture was filtered and the filtrate was concentrated under reduced to give the title compound (600 mg, crude) as a pale yellow oil. LCMS (ES⁺): m/z 524.3 [M+H]⁺.

Synthesis of trans-4-((4-(3-(2,6-dioxopiperidin-3-yl)-1-methyl-1H-indazol-6-yl)piperazin-1-yl)methyl)cyclohexane-1-carboxylic acid. To a solution of trans-tert-butyl-4-((4-(3-(2,6-dioxopiperidin-3-yl)-1-methyl-1H-indazol-6-yl)piperazin-1-yl)methyl)cyclohexane-1-carboxylate (250 mg, 477 μmol, 1.00 eq) in DCM (3 mL) was added TFA (1 mL) at 25 °C. The mixture was stirred at 25 °C for 1 h. LCMS showed trans-tert-butyl-4-((4-(3-(2,6-dioxopiperidin-3-yl)-1-methyl-1H-indazol-6-yl)piperazin-1-yl)methyl)cyclohexane-1-carboxylate was consumed completely and the desired mass was detected. The reaction mixture was concentrated under reduced pressure to give the title compound (220 mg, crude) as a yellow oil. LCMS (ES⁺): m/z 468.2 [M+H]⁺.

Synthesis of trans-(1r,4R)-4-((4-(3-(2,6-dioxopiperidin-3-yl)-1-methyl-1H-indazol-6-yl)piperazin-1-yl)methyl)-N-((1r,4R)-4-((5-fluoro-4-(3-(2-oxopyridin-1(2H)-yl)phenyl)pyrimidin-2-yl)amino)cyclohexyl)cyclohexane-1-carboxamide (Compound 489). To a solution of trans-4-((4-(3-(2,6-dioxopiperidin-3-yl)-1-methyl-1H-indazol-6-yl)piperazin-1-yl)methyl)cyclohexane-1-carboxylic acid (220 mg, 471 μmol, 1.00 eq) in DMF (3 mL) were added DIEA (305 mg, 2.35 mmol, 410 μL, 5.00 eq) and HATU (71.6 mg, 188 μmol, 0.400 eq) at 25 °C. The mixture was stirred at 25 °C for 0.5 h. Then trans-1-[3-[2-[(4-aminocyclohexyl)amino]-5-fluoro-pyrimidin-4-yl]phenyl]pyridin-2-one (92.9 mg, 188 μmol, 0.400 eq, TFA) was added to the reaction mixture at 25 °C. The mixture was stirred at 25 °C for 1 h. LCMS showed trans-4-((4-(3-(2,6-dioxopiperidin-3-yl)-1-methyl-1H-indazol-6-yl)piperazin-1-yl)methyl)cyclohexane-1-carboxylic acid was consumed completely and the desired mass was detected. The reaction was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 31%-61% B over 8.0 min) to give the title compound (Compound 489) (79.0 mg, 92.6 μmol, 19.7% yield, 97.1% purity) as a white solid. LCMS (ES⁺): m/z 829.5 [M+H]⁺.

### Example 471. Synthesis of 1-(2-(4-(3-(2,6-dioxopiperidin-3-yl)-1-methyl-1H-indazol-6-yl)piperidin-1-yl)ethyl)-N-((1r,4r)-4-((5-fluoro-4-(3-(2-oxopyridin-1(2H)-yl)phenyl)pyrimidin-2-yl)amino)cyclohexyl)piperidine-4-carboxamide (Compound 534)

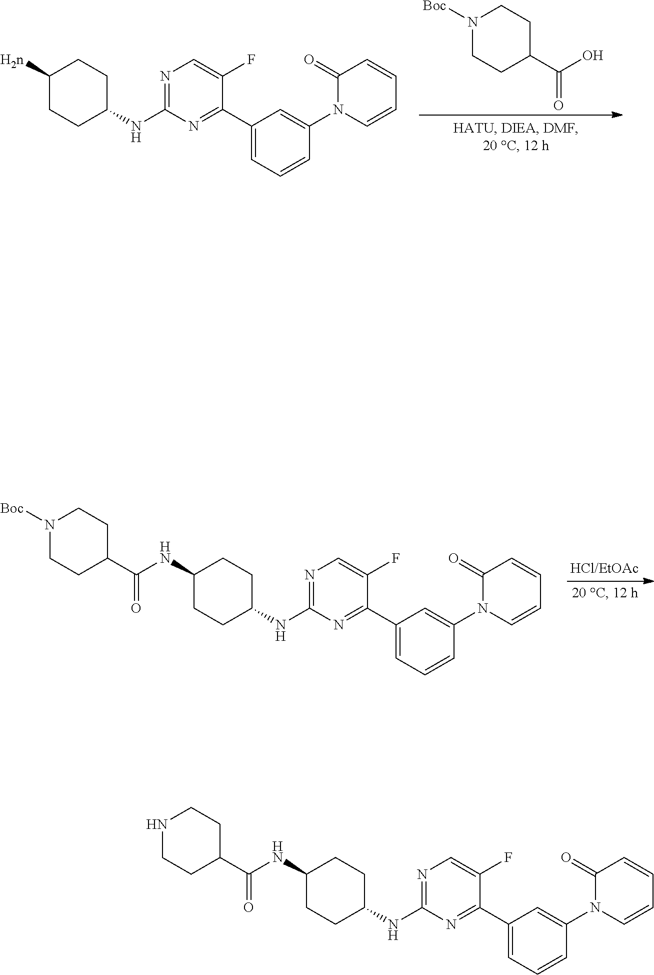

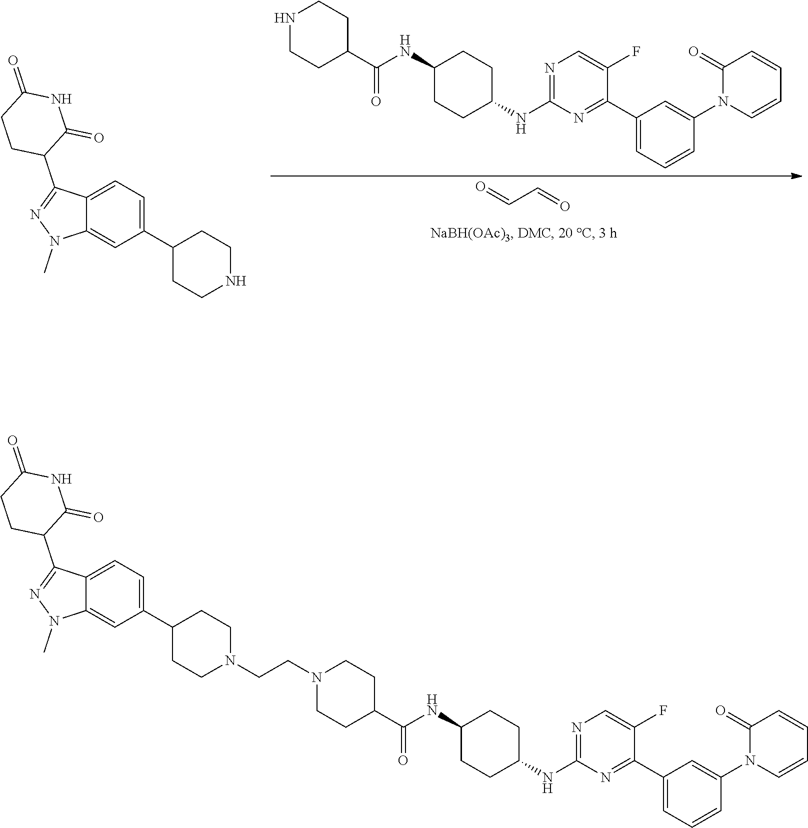

Synthesis of trans-tert-butyl 4-(((1r,4r)-4-((5-fluoro-4-(3-(2-oxopyridin-1(2H)-yl)phenyl)pyrimidin-2-yl)amino)cyclohexyl)carbamoyl)piperidine-1-carboxylate. To a solution of trans-1-(3-(2-(((1r,4r)-4-aminocyclohexyl)amino)-5-fluoropyrimidin-4-yl)phenyl)pyridin-2(1H)-one (2.00 g, 4.05 mmol, 1.00 eq, TFA) in THE (20 mL) were added 1-(tert-butoxycarbonyl)piperidine-4-carboxylic acid (1.12 g, 4.86 mmol, 1.20 eq) and DIEA (2.62 g, 20.3 mmol, 3.53 mL, 5.00 eq) and HATU (4.62 g, 12.2 mmol, 3.00 eq) at 20 °C. The mixture was stirred at 20 °C for 12 h. LC-MS showed that trans-1-(3-(2-(((1r,4r)-4-aminocyclohexyl)amino)-5-fluoropyrimidin-4-yl)phenyl)pyridin-2(1H)-one was consumed completely and one main peak with the desired mass was detected. The mixture was concentrated under reduced pressure. Two batches of crude product, 3.00 g each, were combined to for further purification. The residue was purified by column chromatography (SiO₂, Petroleum ether/Ethyl acetate=1/0 to 0/1) to give the title compound (4.00 g, crude) as a white solid. LCMS (ES⁺): m/z 591.3 [M+H]⁺.

Synthesis of trans-N-((1r,4r)-4-((5-fluoro-4-(3-(2-oxopyridin-1(2H)-yl)phenyl)pyrimidin-2-yl)amino)cyclohexyl)piperidine-4-carboxamide. A solution of trans-tert-butyl 4-(((1r,4r)-4-((5-fluoro-4-(3-(2-oxopyridin-1(2H)-yl)phenyl)pyrimidin-2-yl)amino)cyclohexyl)carbamoyl)piperidine-1-carboxylate (200 mg, 339 μmol, 1.00 eq) in HCl/EtOAc (4 M, 2 mL, 23.6 eq) was stirred at 20 °C for 12 h. LC-MS showed that trans-tert-butyl 4-(((1r,4r)-4-((5-fluoro-4-(3-(2-oxopyridin-1(2H)-yl)phenyl)pyrimidin-2-yl)amino)cyclohexyl)carbamoyl)piperidine-1-carboxylate was consumed completely and one main peak with the desired mass was detected. The mixture was concentrated under reduced pressure to give the title compound (170 mg, crude, HCl) was obtained as a white solid. LCMS (ES⁺): m/z 491.2 [M+H]⁺.

Synthesis of rac-1-(2-(4-(3-(2,6-dioxopiperidin-3-yl)-1-methyl-1H-indazol-6-yl)piperidin-1-yl)ethyl)-N-((1r,4r)-4-((5-fluoro-4-(3-(2-oxopyridin-1(2H)-yl)phenyl)pyrimidin-2-yl)amino)cyclohexyl)piperidine-4-carboxamide (Compound 534). To a solution of 3-(1-methyl-6-(piperidin-4-yl)-1H-indazol-3-yl)piperidine-2,6-dione (70 mg, 193 μmol, 1.00 eq, HCl) in DCM (2 mL) was added trans-N-((1r,4r)-4-((5-fluoro-4-(3-(2-oxopyridin-1(2H)-yl)phenyl)pyrimidin-2-yl)amino)cyclohexyl)piperidine-4-carboxamide (94.6 mg, 180 μmol, 9.31e-1 eq, HCl) and AcOH (11.6 mg, 193 μmol, 11.0 μL, 1.00 eq), oxaldehyde (11.2 mg, 193 μmol, 10.1 μL, 1.00 eq), NaBH(OAc)₃ (123 mg, 579 μmol, 3.00 eq) at 20 °C. The mixture was stirred at 20 °C for 3 h. LC-MS showed that 3-(1-methyl-6-(piperidin-4-yl)-1H-indazol-3-yl)piperidine-2,6-dione was remained and one main peak with the desired mass was detected. The mixture was concentrated under reduced pressure. The residue was purified by prep-HPLC (column: Phenomenex luna C18 100*40 mm*3 um; mobile phase: [H₂O (0.2% FA)-ACN]; gradient: 1%-35% B over 8.0 min) to give the title compound (Compound 534) (4.70 mg, 5.33 μmol, 2.76% yield, 95.7% purity) as a yellow solid. LCMS (ES⁺): m/z 843.4 [M+H]⁺.

### Example 472. Synthesis of trans-1-(2-(4-(3-(2,6-dioxopiperidin-3-yl)-1-methyl-1H-indazol-6-yl)piperazin-1-yl)ethyl)-N-((1r,4r)-4-((5-fluoro-4-(3-(2-oxopyridin-1(2H)-yl)phenyl)pyrimidin-2-yl)amino)cyclohexyl)piperidine-4-carboxamide (Compound 506)

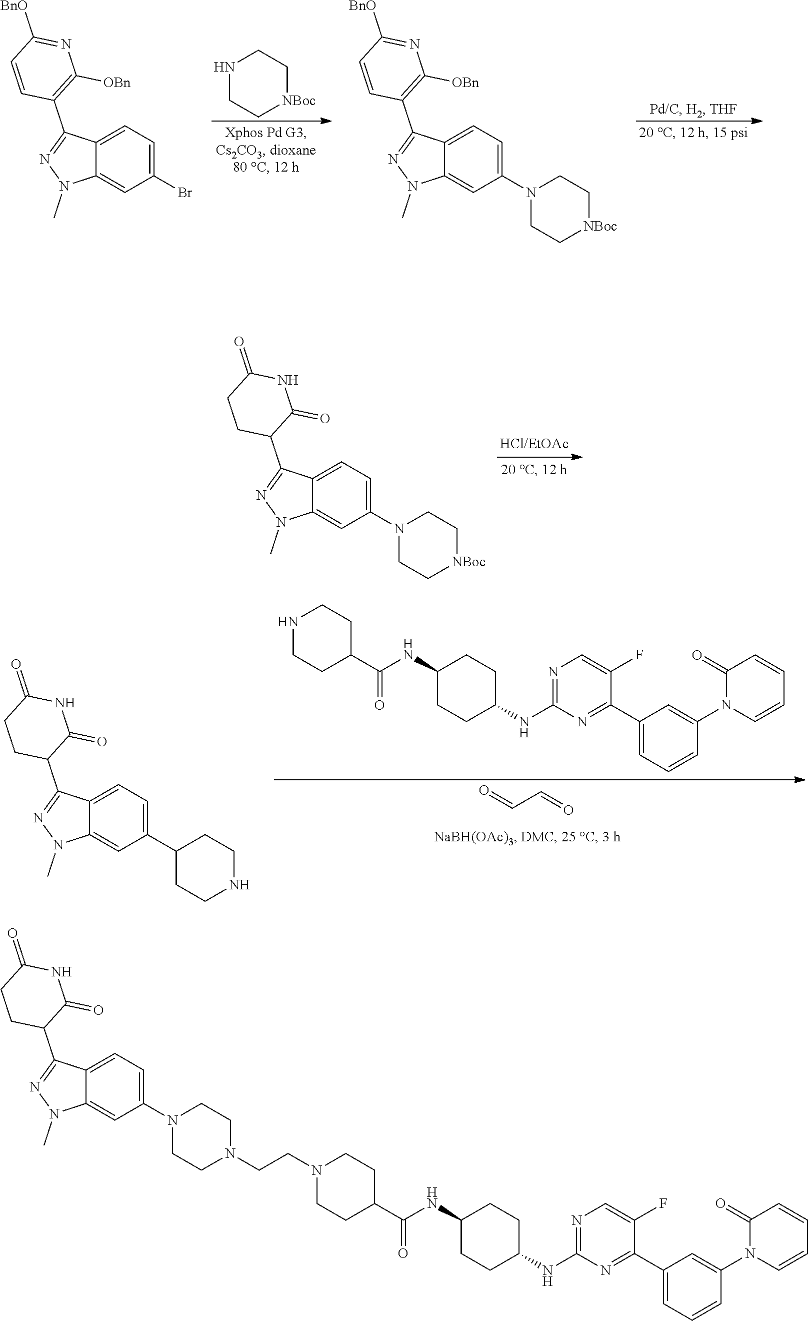

Synthesis of tert-butyl 4-(3-(2,6-bis(benzyloxy)pyridin-3-yl)-1-methyl-1H-indazol-6-yl)piperazine-1-carboxylate. To a solution of 3-(2,6-bis(benzyloxy)pyridin-3-yl)-6-bromo-1-methyl-1H-indazole (2.00 g, 4.00 mmol, 1.00 eq) in dioxane (40 mL) was added tert-butyl piperazine-1-carboxylate (1.12 g, 6.00 mmol, 1.50 eq) and Cs₂CO₃ (2.60 g, 7.99 mmol, 2.00 eq), XPhos Pd G3 (338 mg, 400 μmol, 0.100 eq) at 20 °C. The mixture was stirred at 80 °C for 12 h under N₂. LC-MS showed that 3-(2,6-bis(benzyloxy)pyridin-3-yl)-6-bromo-1-methyl-1H-indazole was consumed completely and one main peak with the desired mass was detected. The mixture was concentrated under reduced pressure. The residue was purified by column chromatography (SiO₂, Petroleum ether:Ethyl acetate=1:0 to 3:1) to give the title compound (2.30 g, 3.80 mmol, 95.0% yield, n/a purity) as a yellow oil. LCMS (ES⁺): m/z 606.3 [M+H]⁺.

Synthesis of tert-butyl 4-(3-(2,6-dioxopiperidin-3-yl)-1-methyl-1H-indazol-6-yl)piperazine-1-carboxylate. To a mixture of 10% Pd/C (2.00 g, 50% purity) in THF (20 mL) was added tert-butyl 4-(3-(2,6-bis(benzyloxy)pyridin-3-yl)-1-methyl-1H-indazol-6-yl)piperazine-1-carboxylate (2.20 g, 3.63 mmol, 1.00 eq) at 20 °C. The mixture was stirred at 20 °C for 12 h under H₂ (15 Psi). LC-MS showed that tert-butyl 4-(3-(2,6-bis(benzyloxy)pyridin-3-yl)-1-methyl-1H-indazol-6-yl)piperazine-1-carboxylate was consumed completely and one main peak with the desired mass was detected. The mixture was filtered and the filtrated was concentrated under reduced pressure to give the title compound (1.50 g, crude) as a yellow solid. LCMS (ES⁺): m/z 428.2 [M+H]⁺.

Synthesis of 3-(1-methyl-6-(piperazin-1-yl)-1H-indazol-3-yl)piperidine-2,6-dione. A solution of tert-butyl 4-(3-(2,6-dioxopiperidin-3-yl)-1-methyl-1H-indazol-6-yl)piperazine-1-carboxylate (100 mg, 234 μmol, 1.00 eq) in HCl/EtOAc (4 M, 4 mL, 68.4 eq) was stirred at 20 °C for 12 h. LC-MS showed that tert-butyl 4-(3-(2,6-dioxopiperidin-3-yl)-1-methyl-1H-indazol-6-yl)piperazine-1-carboxylate was consumed completely and one main peak with the desired mass was detected. The mixture was concentrated under reduced pressure to give the title compound (70 mg, crude, HCl) as a white solid. LCMS (ES⁺): m/z 328.2 [M+H]⁺.

Synthesis of trans-1-(2-(4-(3-(2,6-dioxopiperidin-3-yl)-1-methyl-1H-indazol-6-yl)piperazin-1-yl)ethyl)-N-((1r,4r)-4-((5-fluoro-4-(3-(2-oxopyridin-1(2H)-yl)phenyl)pyrimidin-2-yl)amino)cyclohexyl)piperidine-4-carboxamide (Compound 506). To a solution of 3-(1-methyl-6-(piperazin-1-yl)-1H-indazol-3-yl)piperidine-2,6-dione (70.0 mg, 214 μmol, 1.00 eq) in DCM (2 mL) was added trans-N-((1r,4r)-4-((5-fluoro-4-(3-(2-oxopyridin-1(2H)-yl)phenyl)pyrimidin-2-yl)amino)cyclohexyl)piperidine-4-carboxamide (105 mg, 214 μmol, 1.00 eq) and oxaldehyde (12.4 mg, 214 μmol, 11.2 μL, 1.00 eq), NaBH(OAc)₃ (136 mg, 641 μmol, 3.00 eq) at 20 °C. The mixture was stirred at 20 °C for 3 h. LC-MS showed that 3-(1-methyl-6-(piperazin-1-yl)-1H-indazol-3-yl)piperidine-2,6-dione was remained and one main peak with desired the desired mass was detected. The mixture was concentrated under reduced pressure. The residue was purified by prep-HPLC (column: Xselect CSH C18 100*30 mm*5 um; mobile phase: [H₂O (0.04% HCl)-ACN]; gradient: 10%-50% B over 15.0 min) to give the title compound (Compound 506) (13.1 mg, 14.8 mol, 6.90% yield, 95.1% purity) as a yellow solid. LCMS (ES⁺): m/z 844.4 [M+H]⁺.

### Example 473. Synthesis of trans-2-((1r,4R)-4-(4-(3-(2,6-dioxopiperidin-3-yl)-1-methyl-1H-indazol-6-yl)piperazin-1-yl)cyclohexyl)-N-((1r,4R)-4-((5-fluoro-4-(3-(2-oxopyridin-1(2H)-yl)phenyl)pyrimidin-2-yl)amino)cyclohexyl)acetamide (Compound 505)

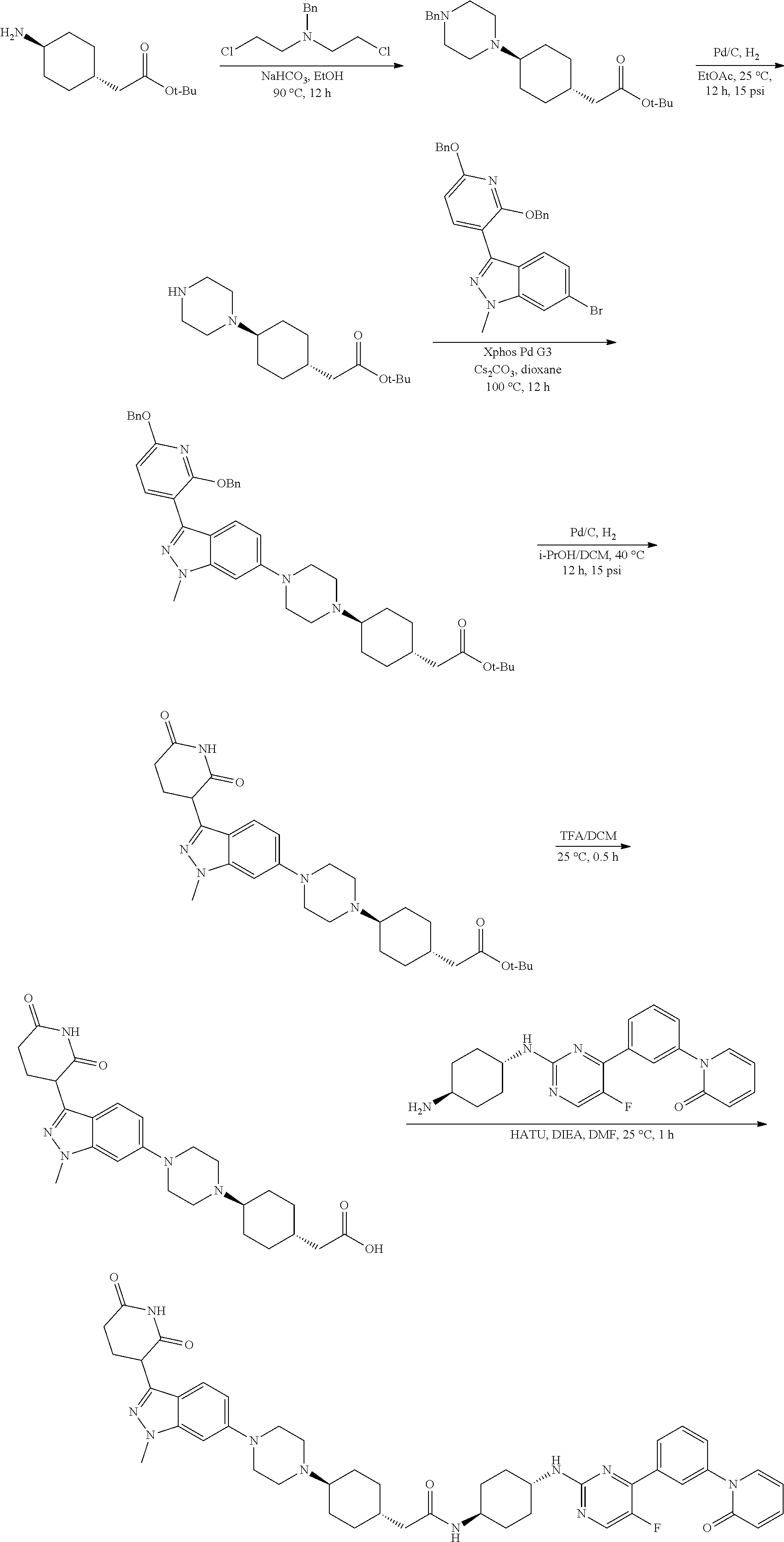

Synthesis trans-tert-butyl 2-(4-(4-benzylpiperazin-1-yl)cyclohexyl)acetate. To a solution of trans-tert-butyl 2-(4-aminocyclohexyl)acetate (5.20 g, 24.4 mmol, 1.00 eq) in EtOH (170 mL) were added NaHCO₃ (5.73 g, 68.3 mmol, 2.66 mL, 2.80 eq) and N-benzyl-2-chloro-N-(2-chloroethyl)ethan-1-amine (6.23 g, 26.8 mmol, 1.10 eq) at 25 °C. The mixture was stirred at 90 °C for 12 h. LC-MS showed trans-tert-butyl 2-(4-aminocyclohexyl)acetate was consumed completely and the desired mass was detected. The reaction mixture was concentrated under reduced pressure to give a residue. The residue was purified by column chromatography (SiO₂, Petroleum ether:Ethyl acetate=5:1 to:1) to give the title compound (5.00 g, 13.4 mmol, 55.1% yield) as a white solid. LCMS (ES⁺): m/z 373.4 [M+H]⁺.

Synthesis of trans-tert-butyl 2-(4-(piperazin-1-yl)cyclohexyl)acetate. 10% Pd/C (4.50 g, 50% purity) was added to the reaction flask at 25 °C under N₂ atmosphere, and then EtOAc (100 mL) were added to the reaction flask, then trans-tert-butyl 2-(4-(4-benzylpiperazin-1-yl)cyclohexyl)acetate (4.50 g, 12.1 mmol, 1.00 eq) was added to the mixture at 25 °C, the suspension was degassed and purged with H₂ for three times. The mixture was stirred at 25 °C for 12 h under H₂ (15 psi) atmosphere. LC-MS showed trans-tert-butyl 2-(4-(4-benzylpiperazin-1-yl)cyclohexyl)acetate was consumed completely and the desired mass was detected. The product was filtered and the filtrate was concentrated under reduced pressure to give the title compound (3.41 g, crude) as a brown oil. LCMS (ES⁺): m/z 283.3 [M+H]⁺.

Synthesis of trans-tert-butyl 2-(4-(4-(3-(2,6-bis(benzyloxy)pyridin-3-yl)-1-methyl-1H-indazol-6-yl)piperazin-1-yl)cyclohexyl)acetate. To a solution of trans-tert-butyl 2-(4-(piperazin-1-yl)cyclohexyl)acetate (3.40 g, 12.0 mmol, 1.00 eq) in dioxane (100 mL) were added 3-(2,6-bis(benzyloxy)pyridin-3-yl)-6-bromo-1-methyl-1H-indazole (6.02 g, 12.0 mmol, 1.00 eq), XPhos Pd G3 (1.02 g, 1.20 mmol, 0.100 eq) and Cs₂CO₃ (7.84 g, 24.1 mmol, 2.00 eq) at 25 °C under N₂ atmosphere. The mixture was stirred at 100 °C for 12 h under N₂ atmosphere. LC-MS showed trans-tert-butyl 2-(4-(piperazin-1-yl)cyclohexyl)acetate was consumed completely and the desired mass was detected. Water (100 ml) was added to the mixture at 25 °C. Then the reaction mixture was extracted with EtOAc (80 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=5:1 to 2:1) to give the title compound (2.70 g, 3.85 mmol, 32.0% yield) as a white solid. LCMS (ES⁺): m/z 702.2 [M+H]⁺.

Synthesis of trans-tert-butyl 2-(4-(4-(3-(2,6-dioxopiperidin-3-yl)-1-methyl-1H-indazol-6-yl)piperazin-1-yl)cyclohexyl)acetate. 10% Pd/C (1.00 g, 50% purity) was added to the reaction flask at 25 °C under N₂ atmosphere, and then i-PrOH (10 mL) and DCM (10 mL) were added to the reaction flask, then trans-tert-butyl 2-(4-(4-(3-(2,6-bis(benzyloxy)pyridin-3-yl)-1-methyl-1H-indazol-6-yl)piperazin-1-yl)cyclohexyl)acetate (1.00 g, 1.42 mmol, 1.00 eq) was added to the mixture at 25 °C, the suspension was degassed and purged with H₂ for three times. The mixture was stirred at 40 °C for 12 h under H₂ (15 psi) atmosphere. LC-MS showed trans-tert-butyl 2-(4-(4-(3-(2,6-bis(benzyloxy)pyridin-3-yl)-1-methyl-1H-indazol-6-yl)piperazin-1-yl)cyclohexyl)acetate was consumed completely and the desired mass was detected. The reaction was filtered and the filtrate was concentrated under reduced pressure to give the title compound (746 mg, crude) as a black oil. LCMS (ES⁺): m/z 524.3 [M+H]⁺.

Synthesis of trans-2-(4-(4-(3-(2,6-dioxopiperidin-3-yl)-1-methyl-1H-indazol-6-yl)piperazin-1-yl)cyclohexyl)acetic acid. To a solution of trans-tert-butyl 2-(4-(4-(3-(2,6-dioxopiperidin-3-yl)-1-methyl-1H-indazol-6-yl)piperazin-1-yl)cyclohexyl)acetate (300 mg, 573 μmol, 1.00 eq) in DCM (3 mL) was added TFA (1 mL) at 25 °C. The mixture was stirred at 25 °C for 0.5 h. LC-MS showed trans-tert-butyl 2-(4-(4-(3-(2,6-dioxopiperidin-3-yl)-1-methyl-1H-indazol-6-yl)piperazin-1-yl)cyclohexyl)acetate was consumed completely and the desired mass was detected. The reaction mixture was concentrated under reduced pressure to remove solvent to give title compound (267 mg, crude) as a brown oil. LCMS (ES⁺): m/z 468.2 [M+H]⁺.

Synthesis of trans-2-((1r,4R)-4-(4-(3-(2,6-dioxopiperidin-3-yl)-1-methyl-1H-indazol-6-yl)piperazin-1-yl)cyclohexyl)-N-((1r,4R)-4-((5-fluoro-4-(3-(2-oxopyridin-1(2H)-yl)phenyl)pyrimidin-2-yl)amino)cyclohexyl)acetamide (Compound 505). To a solution of trans-2-(4-(4-(3-(2,6-dioxopiperidin-3-yl)-1-methyl-1H-indazol-6-yl)piperazin-1-yl)cyclohexyl)acetic acid (267 mg, 571 μmol, 1.00 eq) in DMF (3 mL) were added HATU (86.9 mg, 229 μmol, 0.400 eq), DIEA (221 mg, 1.71 mmol, 298 μL, 3.00 eq) and trans-1-(3-(2-((4-aminocyclohexyl)amino)-5-fluoropyrimidin-4-yl)phenyl)pyridin-2(1H)-one (113 mg, 228 μmol, 0.400 eq, TFA) at 25 °C. The mixture was stirred at 25 °C for 1 h. LC-MS showed trans-2-(4-(4-(3-(2,6-dioxopiperidin-3-yl)-1-methyl-1H-indazol-6-yl)piperazin-1-yl)cyclohexyl)acetic acid was consumed completely and the desired mass was detected. The reaction mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 33%-63% B over 8.0 min) to give title compound (Compound 505) (42.3 mg, 50.6 μmol, 8.86% yield, 99.2% purity) as a off-white solid. LCMS (ES⁺): m/z 829.5 [M+H]⁺

### Example 474. Synthesis of trans-(1r,4R)-4-(4-(4-((2,6-dioxopiperidin-3-yl)amino)-2-fluorophenyl)piperazin-1-yl)-N-((1r,4R)-4-((5-fluoro-4-(3-(2-oxo-1,3-oxazinan-3-yl)phenyl)pyrimidin-2-yl)amino)cyclohexyl)cyclohexane-1-carboxamide (Compound 424)

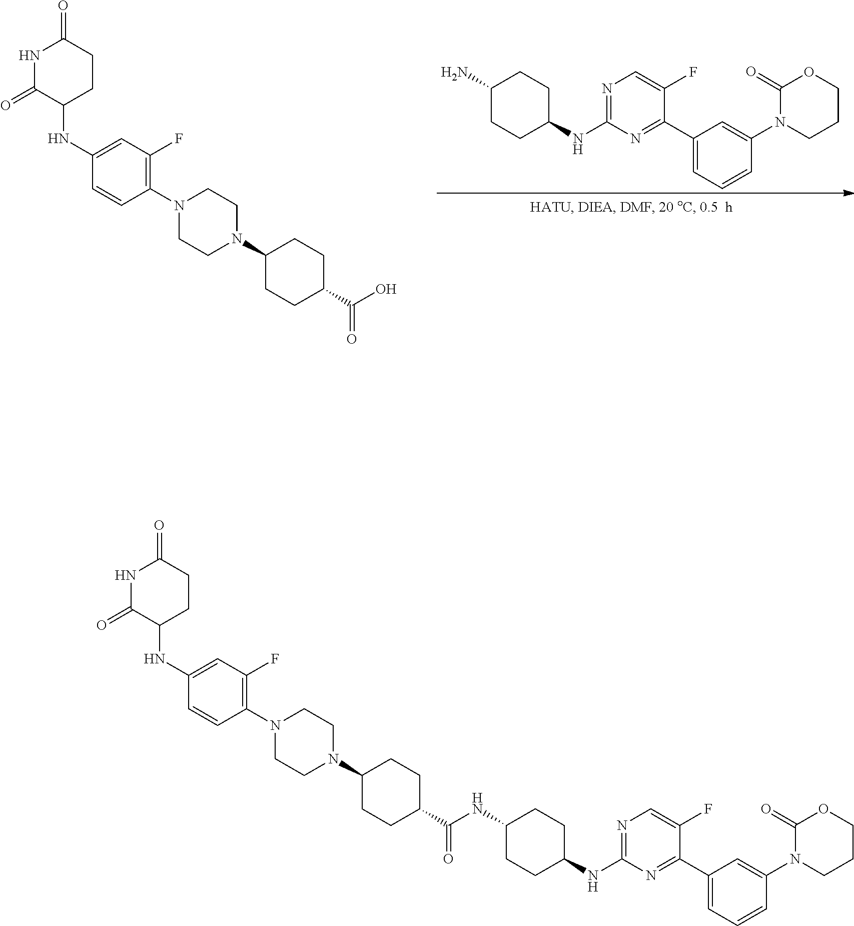 [SMILES: O=C1CCC(Nc2ccc(N3CCN([C@H]4CC[C@H](C(=O)O)CC4)CC3)c(F)c2)C(=O)N1 ; N[C@H]1CC[C@H](Nc2ncc(F)c(-c3cccc(N4CCCOC4=O)c3)n2)CC1 ; O=C1CCC(Nc2ccc(N3CCN([C@H]4CC[C@H](C(=O)N[C@H]5CC[C@H](Nc6ncc(F)c(-c7cccc(N8CCCOC8=O)c7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperazin-1-yl)-N-((1r,4R)-4-((5-fluoro-4-(3-(2-oxo-1,3-oxazinan-3-yl)phenyl)pyrimidin-2-yl)amino)cyclohexyl)cyclohexane-1-carboxamide (Compound 424). To a solution of trans-(1r,4r)-4-(4-(4-((2,6-dioxopiperidin-3-yl)amino)-2-fluorophenyl)piperazin-1-yl)cyclohexane-1-carboxylic acid (1.00 g, 1.92 mmol, 1.00 eq) in DMF (15 mL) were added HATU (219 mg, 576 μmol, 0.300 eq), DIEA (1.24 g, 9.60 mmol, 1.67 mL, 5.00 eq) and trans-3-(3-(2-(((1r,4r)-4-aminocyclohexyl)amino)-5-fluoropyrimidin-4-yl)phenyl)-1,3-oxazinan-2-one (383 mg, 768 μmol, 0.400 eq, TFA) at 20 °C, the mixture was stirred at 20 °C for 0.5 h. LCMS showed trans-(1r,4r)-4-(4-(4-((2,6-dioxopiperidin-3-yl)amino)-2-fluorophenyl)piperazin-1-yl)cyclohexane-1-carboxylic acid was consumed and the desired target MS was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min) to give the title compound (Compound 424) (88.1 mg, 105 μmol, 5.47% yield, 95.4% purity) as a yellow solid. LCMS (ES⁺): m/z 800.3 [M+H]⁺

### Example 475. Synthesis of trans-(1R,4R)-4-(4-(4-(((R)-2,6-dioxopiperidin-3-yl)amino)-2-fluorophenyl)piperazin-1-yl)-N-((1r,4R)-4-((5-fluoro-4-(3-(2-oxo-1,3-oxazinan-3-yl)phenyl)pyrimidin-2-yl)amino)cyclohexyl)cyclohexane-1-carboxamide (Compound 461) and trans-(1S,4R)-4-(4-(4-(((S)-2,6-dioxopiperidin-3-yl)amino)-2-fluorophenyl)piperazin-1-yl)-N-((1r,4R)-4-((5-fluoro-4-(3-(2-oxo-1,3-oxazinan-3-yl)phenyl)pyrimidin-2-yl)amino)cyclohexyl)cyclohexane-1-carboxamide (Compound 462) (The Two Isomers were Assigned Arbitrarily)

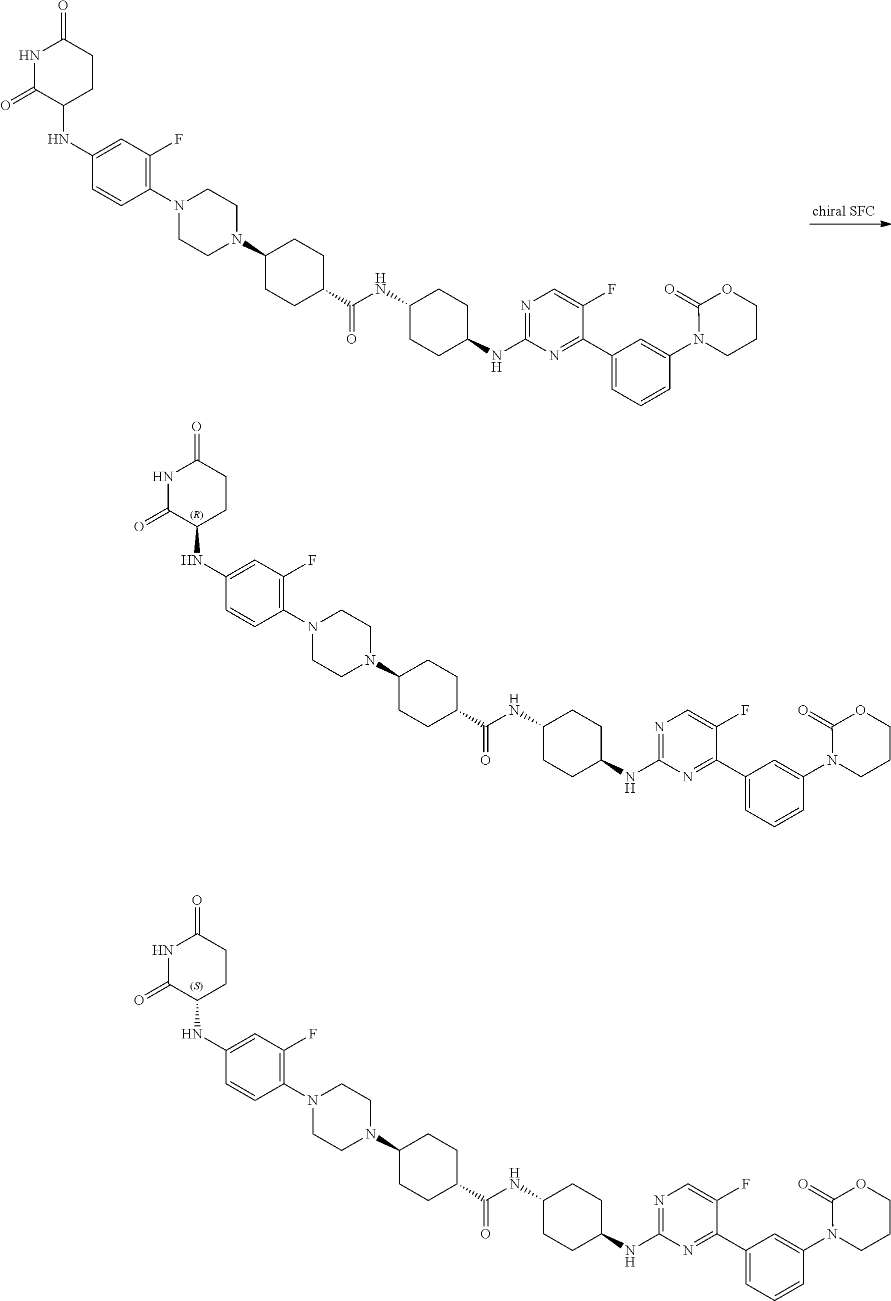

Synthesis of trans-(1R,4R)-4-(4-(4-(((R)-2,6-dioxopiperidin-3-yl)amino)-2-fluorophenyl)piperazin-1-yl)-N-((1r,4R)-4-((5-fluoro-4-(3-(2-oxo-1,3-oxazinan-3-yl)phenyl)pyrimidin-2-yl)amino)cyclohexyl)cyclohexane-1-carboxamide (Compound 461) and trans-(1S,4R)-4-(4-(4-(((S)-2,6-dioxopiperidin-3-yl)amino)-2-fluorophenyl)piperazin-1-yl)-N-((1r,4R)-4-((5-fluoro-4-(3-(2-oxo-1,3-oxazinan-3-yl)phenyl)pyrimidin-2-yl)amino)cyclohexyl)cyclohexane-1-carboxamide (Compound 462) (the two isomers were assigned arbitrarily). Trans-(1r,4R)-4-(4-(4-((2,6-dioxopiperidin-3-yl)amino)-2-fluorophenyl)piperazin-1-yl)-N-((1r,4R)-4-((5-fluoro-4-(3-(2-oxo-1,3-oxazinan-3-yl)phenyl)pyrimidin-2-yl)amino)cyclohexyl)cyclohexane-1-carboxamide (Compound 424) (83.1 mg, 104 μmol, 1.00 eq) was separated by chiral SFC (column: ChiralPak IH, 250*30 mm, 10 um; mobile phase: [CO₂—IPA (0.1% IPAm)]; B %: 56%, isocratic elution mode) to give the title compound (Compound 461) (8.90 mg, 11.1 μmol, 10.7% yield, 100% purity, 100% ee) (retention time: 1.248, peak 1) as a white solid. LCMS (ES⁺): m/z 800.3 [M+H]⁺ and the title compound (Compound 462) (21.4 mg, 26.4 μmol, 25.4% yield, 98.5% purity, 99.5% ee) (retention time: 2.081, peak 2) as a pale yellow solid. LCMS (ES⁺): m/z 800.4 [M+H]⁺

### Example 476. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(3-(2-oxo-1,3-oxazinan-3-yl)phenyl)pyrimidin-2-yl)amino)cyclohexyl)cyclohexane-1-carboxamide (Compound 576)

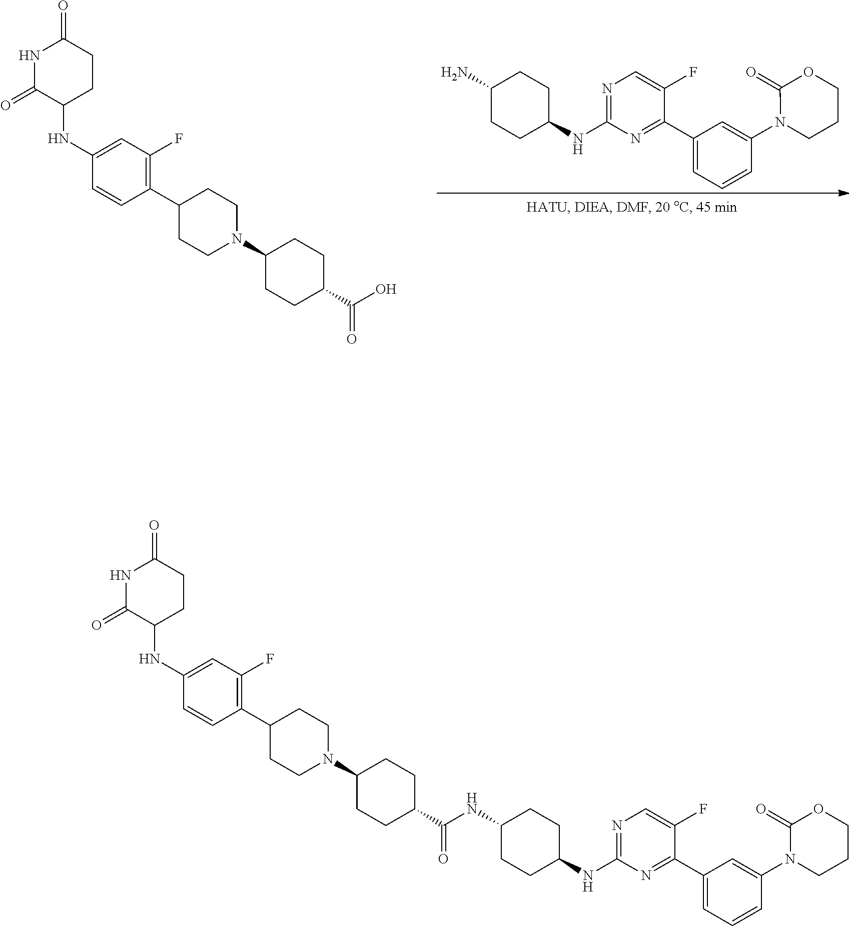 [SMILES: O=C1CCC(Nc2ccc(C3CCN([C@H]4CC[C@H](C(=O)O)CC4)CC3)c(F)c2)C(=O)N1 ; N[C@H]1CC[C@H](Nc2ncc(F)c(-c3cccc(N4CCCOC4=O)c3)n2)CC1 ; O=C1CCC(Nc2ccc(C3CCN([C@H]4CC[C@H](C(=O)N[C@H]5CC[C@H](Nc6ncc(F)c(-c7cccc(N8CCCOC8=O)c7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(3-(2-oxo-1,3-oxazinan-3-yl)phenyl)pyrimidin-2-yl)amino)cyclohexyl)cyclohexane-1-carboxamide (Compound 576). To a solution of trans-(1r,4r)-4-(4-(4-((2,6-dioxopiperidin-3-yl)amino)-2-fluorophenyl)piperidin-1-yl)cyclohexane-1-carboxylic acid (250 mg, 579 μmol, 1.00 eq) in DMF (3 mL) were added HATU (77.1 mg, 203 μmol, 0.350 eq) and DIEA (225 mg, 1.74 mmol, 303 μL, 3.00 eq) at 20 °C, then trans-3-(3-(2-(((1r,4r)-4-aminocyclohexyl)amino)-5-fluoropyrimidin-4-yl)phenyl)-1,3-oxazinan-2-one (116 mg, 232 μmol, 0.400 eq, TFA) was added to the mixture, the mixture was stirred at 20 °C for 30 min. LCMS showed trans-(1r,4r)-4-(4-(4-((2,6-dioxopiperidin-3-yl)amino)-2-fluorophenyl)piperidin-1-yl)cyclohexane-1-carboxylic acid remained and the desired target MS was detected. Then HATU (55.1 mg, 145 μmol, 0.250 eq) was added to the mixture at 20 °C, the mixture was stirred at 20 °C for another 15 min. LCMS showed trans-(1r,4r)-4-(4-(4-((2,6-dioxopiperidin-3-yl)amino)-2-fluorophenyl)piperidin-1-yl)cyclohexane-1-carboxylic acid was consumed completely and the desired target MS was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 20%-50% B over 8.0 min) to give the title compound (Compound 576) (46.0 mg, 53.4 μmol, 9.21% yield, 92.7% purity) as a white solid. LCMS (ES⁺): m/z 799.4 [M+H]+Example 477. Synthesis of trans-1′-(4-((2,6-dioxopiperidin-3-yl)amino)-2-fluorophenyl)-N-(4-((5-fluoro-4-(3-(2-oxo-1,3-oxazinan-3-yl)phenyl)pyrimidin-2-yl)amino)cyclohexyl)-[1,4′-bipiperidine]-4-carboxamide (Compound 597)

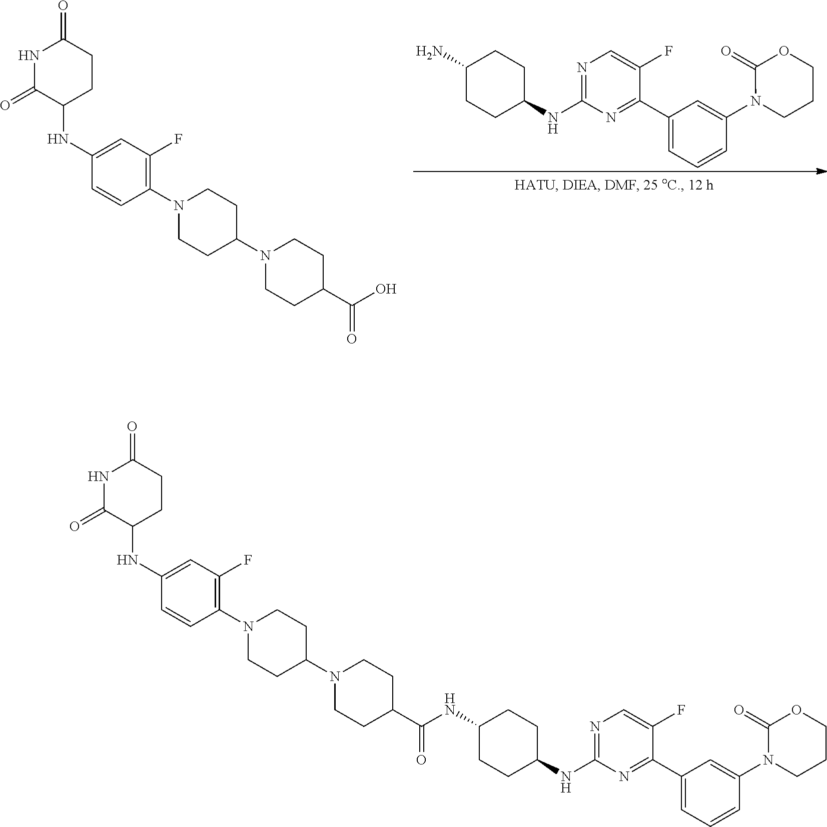 [SMILES: O=C1CCC(Nc2ccc(N3CCC(N4CCC(C(=O)O)CC4)CC3)c(F)c2)C(=O)N1 ; N[C@H]1CC[C@H](Nc2ncc(F)c(-c3cccc(N4CCCOC4=O)c3)n2)CC1 ; O=C1CCC(Nc2ccc(N3CCC(N4CCC(C(=O)N[C@H]5CC[C@H](Nc6ncc(F)c(-c7cccc(N8CCCOC8=O)c7)n6)CC5)CC4)CC3)c(F)c2)C(=O)N1]

Synthesis of trans-1′-(4-((2,6-dioxopiperidin-3-yl)amino)-2-fluorophenyl)-N-(4-((5-fluoro-4-(3-(2-oxo-1,3-oxazinan-3-yl)phenyl)pyrimidin-2-yl)amino)cyclohexyl)-[1,4′-bipiperidine]-4-carboxamide (Compound 597). To a solution of 1′-(4-((2,6-dioxopiperidin-3-yl)amino)-2-fluorophenyl)-[1,4′-bipiperidine]-4-carboxylic acid (500 mg, 914 μmol, 1.00 eq, TFA) in DMF (3 mL) was added DIEA (354 mg, 2.74 mmol, 478.07 μL, 3.00 eq), trans-3-(3-(2-((4-aminocyclohexyl)amino)-5-fluoropyrimidin-4-yl)phenyl)-1,3-oxazinan-2-one (246 mg, 585 μmol, 0.64 eq, HCl) and HATU (521 mg, 1.37 mmol, 1.50 eq) at 25 °C. The mixture was stirred at 25 °C for 12 h. LC-MS showed desired mass was detected. The reaction mixture was concentrated under reduced pressure. The residue was purified by prep-HPLC (column: Phenomenex Gemini-NX 80*40 mm*3 um; mobile phase: [H₂O (10 mM NH₄HCO₃)-ACN]; gradient: 20%-50% B over 8.0 min) to give the title compound (Compound 597) (107 mg, 127 μmol, 13.9% yield, 94.7% purity) as a pale yellow solid. LCMS (ES⁺): m/z 800.4 [M+H]⁺.

### Example 478. Synthesis of 1′-(4-(((R)-2,6-dioxopiperidin-3-yl)amino)-2-fluorophenyl)-N-((trans)-4-((5-fluoro-4-(3-(2-oxo-1,3-oxazinan-3-yl)phenyl)pyrimidin-2-yl)amino)cyclohexyl)-[1,4′-bipiperidine]-4-carboxamide (Compound 543) and 1′-(4-(((S)-2,6-dioxopiperidin-3-yl)amino)-2-fluorophenyl)-N-((trans)-4-((5-fluoro-4-(3-(2-oxo-1,3-oxazinan-3-yl)phenyl)pyrimidin-2-yl)amino)cyclohexyl)-[1,4′-bipiperidine]-4-carboxamide (Compound 542)

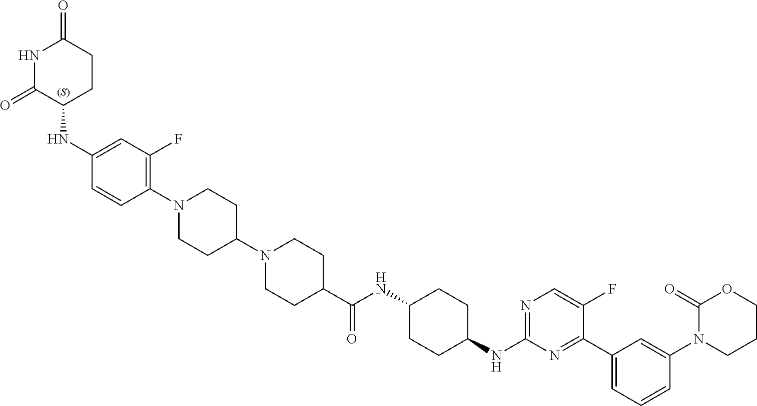

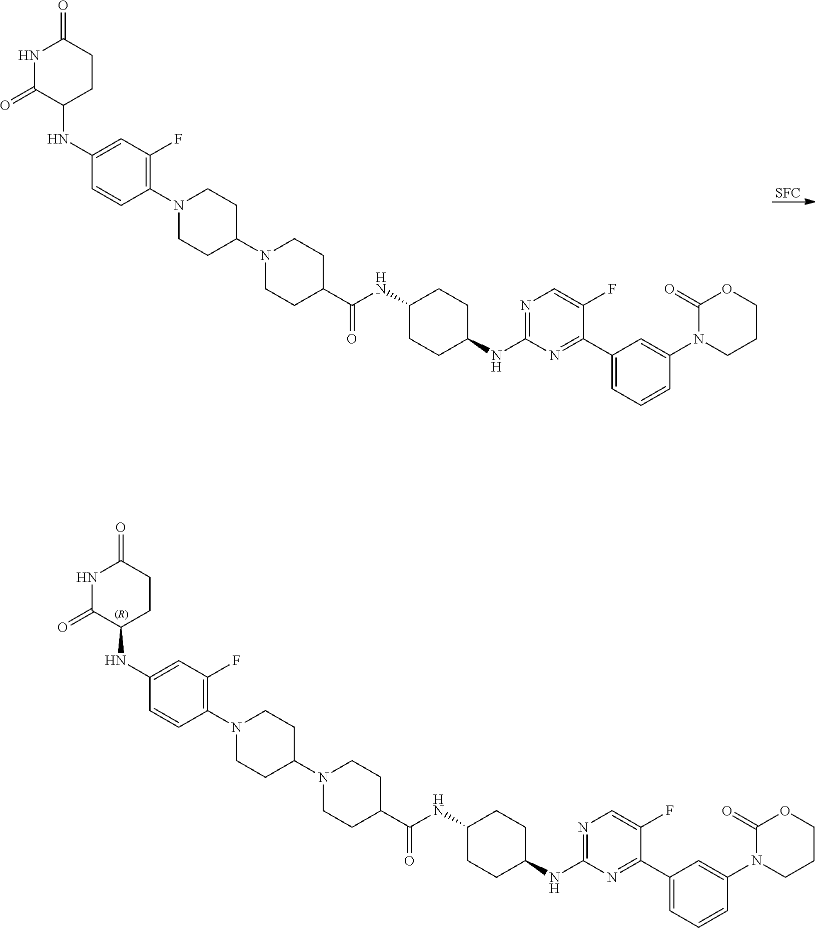

Synthesis of 1′-(4-(((R)-2,6-dioxopiperidin-3-yl)amino)-2-fluorophenyl)-N-((trans)-4-((5-fluoro-4-(3-(2-oxo-1,3-oxazinan-3-yl)phenyl)pyrimidin-2-yl)amino)cyclohexyl)-[1,4′-bipiperidine]-4-carboxamide (Compound 543) and 1′-(4-(((S)-2,6-dioxopiperidin-3-yl)amino)-2-fluorophenyl)-N-((trans)-4-((5-fluoro-4-(3-(2-oxo-1,3-oxazinan-3-yl)phenyl)pyrimidin-2-yl)amino)cyclohexyl)-[1,4′-bipiperidine]-4-carboxamide (Compound 542). trans-1′-(4-((2,6-dioxopiperidin-3-yl)amino)-2-fluorophenyl)-N-(4-((5-fluoro-4-(3-(2-oxo-1,3-oxazinan-3-yl)phenyl)pyrimidin-2-yl)amino)cyclohexyl)-[1,4′-bipiperidine]-4-carboxamide (Compound 597) was separated by SFC (column: ChiralPak IH, 250*30 mm, 10 um; mobile phase: [CO₂-IPA:CAN=1:1]; B %: 65%, isocratic elution mode) to give the title compound (Compound 543) (25.9 mg, 32.3 μmol, 25.9% yield, 100% purity) as a pale yellow solid. LCMS (ES⁺): m/z 800.4 [M+H]⁺ and the title compound (Compound 542) (23.5 mg, 29.2 μmol, 23.4% yield, 99.5% purity) as a pale yellow solid. LCMS (ES⁺): m/z 800.4 [M+H]⁺.

### Example 479. Synthesis of trans-2-((1r,4R)-4-((4-(4-((2,6-dioxopiperidin-3-yl)amino)-2-fluorophenyl)piperazin-1-yl)methyl)cyclohexyl)-N-((1r,4R)-4-((5-fluoro-4-(3-(2-oxo-1,3-oxazinan-3-yl)phenyl)pyrimidin-2-yl)amino)cyclohexyl)acetamide (Compound 561)

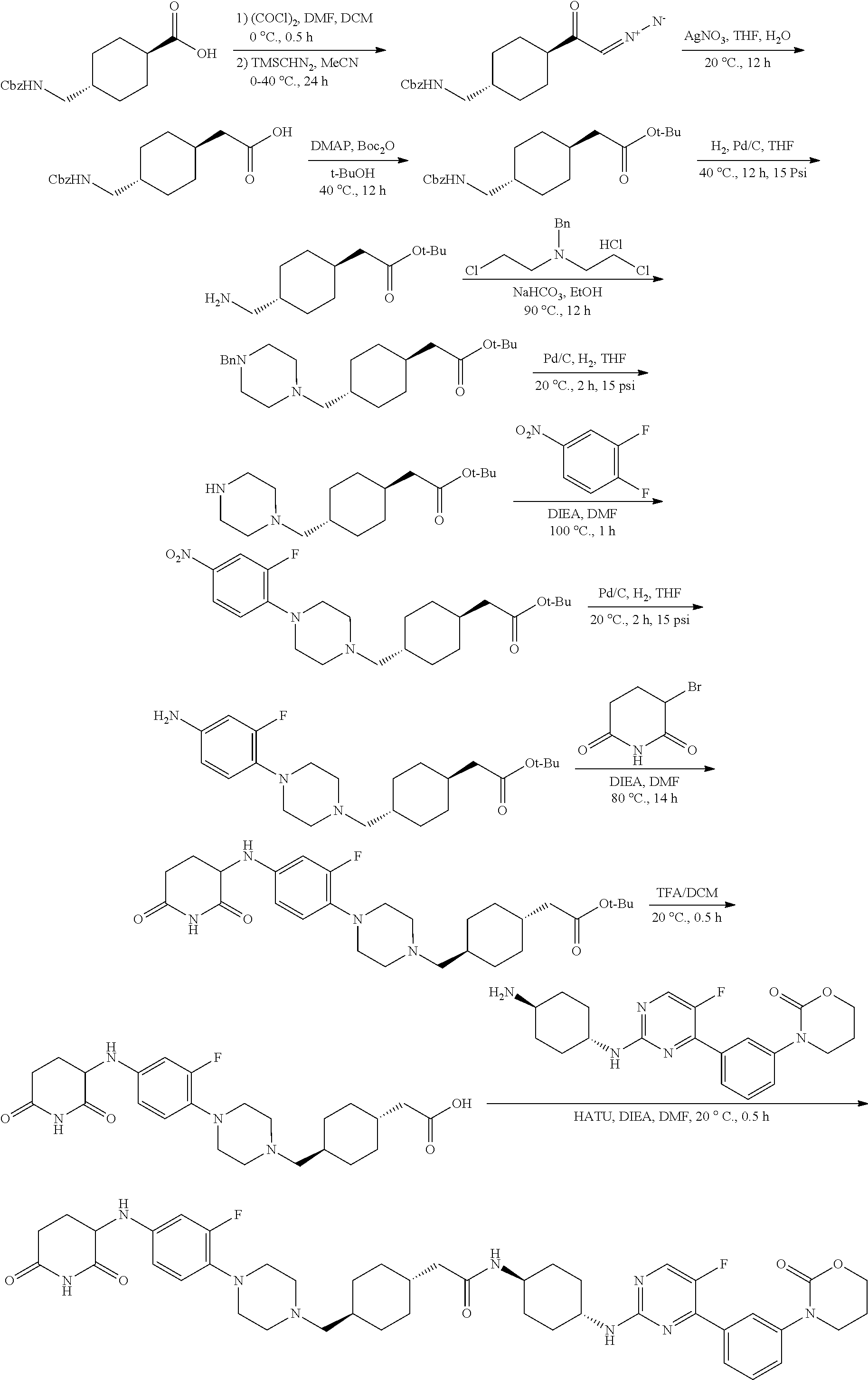

Synthesis of trans-benzyl ((4-(2-diazoacetyl)cyclohexyl)methyl)carbamate. To a solution of 4-(benzyloxycarbonylaminomethyl)cyclohexanecarboxylic acid (20.0 g, 68.7 mmol, 1.00 eq) in DCM (250 mL) was added DMF (502 mg, 6.86 mmol, 528 μL, 0.100 eq) and oxalyl dichloride (9.58 g, 75.5 mmol, 6.61 mL, 1.10 eq) at 0 °C, then the mixture was stirred at 0 °C for 0.5 h under N₂, the mixture was concentrated under reduced pressure to give a residue. Then the residue was dissolved in CH₃CN (500 mL) and TMSCHN₂ (2 M, 41.2 mL, 1.20 eq) at 20 °C, the mixture was stirred at 40 °C for 24 h under N₂. LCMS showed the desired target MS was detected. The mixture was concentrated under reduced pressure to give the title compound (21 g, crude) as a yellow oil. LCMS (ES⁺): m/z 316.2 [M+H]⁺

Synthesis of trans-2-(4-((((benzyloxy)carbonyl)amino)methyl)cyclohexyl)acetic acid. AgNO₃ (13.7 g, 80.5 mmol, 1.21 eq) was dissolved in THF (500 mL) and H₂O (100 mL) at 20 °C, then trans-benzyl ((4-(2-diazoacetyl)cyclohexyl)methyl)carbamate (21.0 g, 66.6 mmol, 1.00 eq) was added to the mixture at 20 °C, the resulting mixture was stirred at 20 °C for 12 h. LCMS showed trans-benzyl ((4-(2-diazoacetyl)cyclohexyl)methyl)carbamate was consumed and the desired target MS was detected. The mixture was concentrated under reduced pressure to give remove THF. Then the remaining aqueous layer was alkalified by NaOH (1 M) to adjust pH to 9 and washed with EtOAc (500 mL*3). The aqueous phase was adjust pH to 2-3 with HCl (1 M). Then the above mixture was extracted with EtOAc (500 mL*3). The combined organic layers were dried over Na₂SO₄, filtered and the filtrate was concentrated under reduced pressure to give the title compound (11.0 g, crude) as a white solid. LCMS (ES⁺): m/z 306.3 [M+H]⁺

Synthesis of trans-tert-butyl 2-(4-((((benzyloxy)carbonyl)amino)methyl)cyclohexyl)acetate. To a solution of trans-2-(4-((((benzyloxy)carbonyl)amino)methyl)cyclohexyl)acetic acid (11.0 g, 36.0 mmol, 1.00 eq) in t-BuOH (200 mL) were added Boc₂O (15.7 g, 72.0 mmol, 16.6 mL, 2.00 eq) and DMAP (1.32 g, 10.81 mmol, 0.300 eq) at 20 °C, then the mixture was stirred at 40 °C for 12 h. LCMS showed trans-2-(4-((((benzyloxy)carbonyl)amino)methyl)cyclohexyl)acetic acid was consumed and the desired target MS was detected. The mixture was concentrated under reduced pressure to give a residue, then H₂O (200 mL) was added to the residue and the mixture was extracted with EtOAc (200 mL*3), the combined organic layers were washed with brine (200 mL*3), dried over Na₂SO₄, filtered and the filtrate was concentrated under reduced pressure to give a residue. The residue was purified by MPLC (SiO₂, PE/EtOAc=1/0 to 0/1) to give the title compound (9.50 g, 26.3 mmol, 73.0% yield) as a colorless oil. LCMS (ES⁺): m/z 306.3 [M+H−56]⁺

Synthesis of trans-tert-butyl 2-(4-(aminomethyl)cyclohexyl)acetate. 10% Pd/C (5.00 g, 50% purity) was added to THF (200 mL) at 20 °C under N₂ atmosphere, then trans-tert-butyl 2-(4-((((benzyloxy)carbonyl)amino)methyl)cyclohexyl)acetate (9.50 g, 26.3 mmol, 1.00 eq) was added to the mixture at 20 °C and the mixture was degassed and purged with H₂ for three times. The mixture was stirred at 40 °C for 12 h under H₂ (15 psi), LCMS showed trans-tert-butyl 2-(4-((((benzyloxy)carbonyl)amino)methyl)cyclohexyl)acetate was consumed and the desired target MS was detected. The mixture were filtered and the filtrate was concentrated under reduced pressure to give the title compound (6.00 g, crude) as a colorless oil. LCMS (ES⁺): m/z 228.4 [M+H]⁺

Synthesis of trans-tert-butyl 2-(4-((4-benzylpiperazin-1-yl)methyl)cyclohexyl)acetate. To a solution of trans-tert-butyl 2-(4-(aminomethyl)cyclohexyl)acetate (5.00 g, 22.0 mmol, 1.00 eq) in EtOH (100 mL) were added N-benzyl-2-chloro-N-(2-chloroethyl)ethanamine (5.90 g, 22.0 mmol, 1.00 eq, HCl) and NaHCO₃ (4.62 g, 55.0 mmol, 2.14 mL, 2.50 eq) at 20 °C. The mixture was stirred at 90 °C for 12 h. LCMS showed trans-tert-butyl 2-(4-(aminomethyl)cyclohexyl)acetate was consumed and the desired mass was detected. The mixture was filtered and the filtrate was concentrated reduced pressure to give a residue, H₂O (100 mL) was added to the residue and the mixture was extracted with EtOAc (50 mL*3). The combined organic layers were washed with saturated aqueous NaCl solution (50 mL*3), dried over Na₂SO₄, filtered and the filtrate was concentrated reduced pressure to give a residue. The residue was purified by MPLC (SiO₂, PE/EtOAc=1/0 to 0/1) to give the title compound (4.20 g, 10.9 mmol, 49.4% yield) as a brown oil. LCMS (ES⁺): m/z 387.6 [M+H]⁺

Synthesis of trans-tert-butyl 2-(4-(piperazin-1-ylmethyl)cyclohexyl)acetate. 10% Pd/C (500 mg, 50% purity) was added to THE (100 mL) at 20 °C under N₂ atmosphere, then trans-tert-butyl 2-(4-((4-benzylpiperazin-1-yl)methyl)cyclohexyl)acetate (4.20 g, 10.9 mmol, 1.00 eq) was added at 20 °C, the suspension was degassed and purged with H₂ for three times. The mixture was stirred at 20 °C for 2 h under H₂ (15 psi). LCMS showed trans-tert-butyl 2-(4-((4-benzylpiperazin-1-yl)methyl)cyclohexyl)acetate was consumed and the desired mass was detected. The mixture was filtered and the filtrate was concentrated under reduced pressure to give the title compound (3.20 g, crude) as a yellow oil. LCMS (ES⁺): m/z 297.2 [M+H]⁺

Synthesis of trans-tert-butyl 2-(4-((4-(2-fluoro-4-nitrophenyl)piperazin-1-yl)methyl)cyclohexyl)acetate. To a solution of trans-tert-butyl 2-(4-(piperazin-1-ylmethyl)cyclohexyl)acetate (2.80 g, 9.45 mmol, 1.00 eq) and 1,2-difluoro-4-nitrobenzene (1.50 g, 9.45 mmol, 1.05 mL, 1.00 eq) in DMF (50 mL) was added DIEA (3.66 g, 28.3 mmol, 4.94 mL, 3.00 eq) at 20 °C. The mixture was stirred at 100 °C for 1 h. LCMS showed trans-tert-butyl 2-(4-(piperazin-1-ylmethyl)cyclohexyl)acetate was consumed and the desired target MS was detected. H₂O (50 mL) was added to the mixture, then the mixture was filtered. The filter cake was dried under reduced pressure to give the title compound (3.50 g, crude) as a yellow solid. LCMS (ES⁺): m/z 436.2 [M+H]⁺

Synthesis of trans-tert-butyl 2-(4-((4-(4-amino-2-fluorophenyl)piperazin-1-yl)methyl)cyclohexyl)acetate. 10% Pd/C (2.00 g, 50.0% purity) was added to THF (80 mL) at 20 °C under N₂ atmosphere, then trans-tert-butyl 2-(4-((4-(2-fluoro-4-nitrophenyl)piperazin-1-yl)methyl)cyclohexyl)acetate (3.50 g, 8.04 mmol, 1.00 eq) was added at 20 °C, the suspension was degassed and purged with H₂ for three times. The mixture was stirred at 20 °C for 2 h under H₂ (15 psi). LCMS showed trans-tert-butyl 2-(4-((4-(2-fluoro-4-nitrophenyl)piperazin-1-yl)methyl)cyclohexyl)acetate was consumed and the desired target MS was detected. The mixture was filtered and the filtrate was concentrated under reduced pressure to give the title compound (3.20 g, crude) as a colorless oil. LCMS (ES⁺): m/z 406.3 [M+H]⁺

Synthesis of trans-tert-butyl 2-(4-((4-(4-((2,6-dioxopiperidin-3-yl)amino)-2-fluorophenyl)piperazin-1-yl)methyl)cyclohexyl)acetate. To a solution of trans-tert-butyl 2-(4-((4-(4-amino-2-fluorophenyl)piperazin-1-yl)methyl)cyclohexyl)acetate (3.20 g, 7.89 mmol, 1.00 eq) and 3-bromopiperidine-2,6-dione (3.03 g, 15.8 mmol, 2.00 eq) in DMF (60 mL) was added DIEA (3.06 g, 23.7 mmol, 4.12 mL, 3.00 eq) at 20 °C, the mixture was stirred at 80 °C for 14 h. LCMS showed trans-tert-butyl 2-(4-((4-(4-amino-2-fluorophenyl)piperazin-1-yl)methyl)cyclohexyl)acetate was consumed and the desired target MS was detected. H₂O (100 mL) was added to the mixture and the mixture was extracted with EtOAc (50 mL*3). The combined organic layers were washed with brine (50 mL*3), dried over Na₂SO₄, filtered and the filtrate was concentrated under reduced pressure to give a residue. The residue was purified by MPLC (SiO₂, PE/EtOAc=1/0 to 1/1) to give the title compound (1.90 g, 3.68 mmol, 46.6% yield) as a yellow oil. LCMS (ES⁺): m/z 517.3 [M+H]⁺

Synthesis of trans-2-(4-((4-(4-((2,6-dioxopiperidin-3-yl)amino)-2-fluorophenyl)piperazin-1-yl)methyl)cyclohexyl)acetic acid. A solution of trans-tert-butyl 2-(4-((4-(4-((2,6-dioxopiperidin-3-yl)amino)-2-fluorophenyl)piperazin-1-yl)methyl)cyclohexyl)acetate (1.00 g, 1.94 mmol, 1.00 eq) in DCM (9 mL) and TFA (3 mL) was stirred at 20 °C for 0.5 h. LCMS showed trans-tert-butyl 2-(4-((4-(4-((2,6-dioxopiperidin-3-yl)amino)-2-fluorophenyl)piperazin-1-yl)methyl)cyclohexyl)acetate was consumed and the desired target MS was detected. The mixture was concentrated under reduced pressure to give the title compound (890 mg, crude) as a yellow oil. LCMS (ES⁺): m/z 461.2 [M+H]⁺

Synthesis of trans-2-((1r,4R)-4-((4-(4-((2,6-dioxopiperidin-3-yl)amino)-2-fluorophenyl)piperazin-1-yl)methyl)cyclohexyl)-N-((1r,4R)-4-((5-fluoro-4-(3-(2-oxo-1,3-oxazinan-3-yl)phenyl)pyrimidin-2-yl)amino)cyclohexyl)acetamide (Compound 561). To a solution of trans-2-(4-((4-(4-((2,6-dioxopiperidin-3-yl)amino)-2-fluorophenyl)piperazin-1-yl)methyl)cyclohexyl)acetic acid (445 mg, 966 μmol, 1.00 eq) in DMF (8 mL) were added HATU (129 mg, 338 μmol, 0.350 eq) and DIEA (375 mg, 2.90 mmol, 505 μL, 3.00 eq) at 20 °C. Then to the mixture was added trans-3-(3-(2-(((1r,4r)-4-aminocyclohexyl)amino)-5-fluoropyrimidin-4-yl)phenyl)-1,3-oxazinan-2-one (290 mg, 580 μmol, 0.600 eq, TFA) and the resulting mixture was stirred at 20 °C for 0.5 h. LCMS showed trans-2-(4-((4-(4-((2,6-dioxopiperidin-3-yl)amino)-2-fluorophenyl)piperazin-1-yl)methyl)cyclohexyl)acetic acid was consumed and the desired target MS was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 35%-70% B over 8.0 min) to give the title compound (Compound 561) (137 mg, 162 μmol, 16.8% yield, 97.6% purity) as a white solid. LCMS (ES⁺): m/z 828.4 [M+H]⁺

### Example 480. Synthesis of 1′-(4-((2,6-dioxopiperidin-3-yl)amino)-2-fluorophenyl)-3′-fluoro-N-((1r,4r)-4-((5-fluoro-4-(3-(2-oxo-1,3-oxazinan-3-yl)phenyl)pyrimidin-2-yl)amino)cyclohexyl)-[1,4′-bipiperidine]-4-carboxamide (Compound 525)

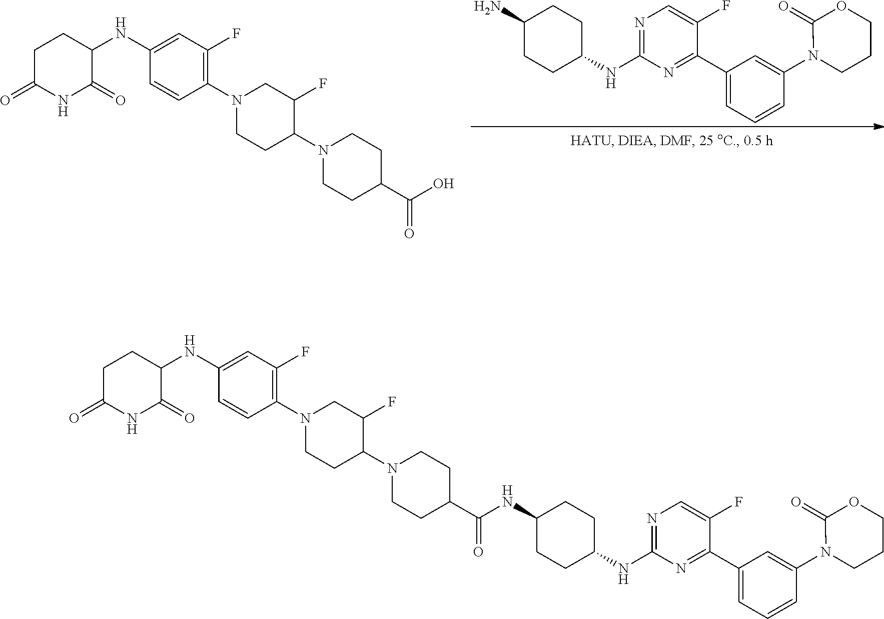 [SMILES: O=C1CCC(Nc2ccc(N3CCC(N4CCC(C(=O)O)CC4)C(F)C3)c(F)c2)C(=O)N1 ; N[C@H]1CC[C@H](Nc2ncc(F)c(-c3cccc(N4CCCOC4=O)c3)n2)CC1 ; O=C1CCC(Nc2ccc(N3CCC(N4CCC(C(=O)N[C@H]5CC[C@H](Nc6ncc(F)c(-c7cccc(N8CCCOC8=O)c7)n6)CC5)CC4)C(F)C3)c(F)c2)C(=O)N1]

Synthesis of 1′-(4-((2,6-dioxopiperidin-3-yl)amino)-2-fluorophenyl)-3′-fluoro-N-((1r,4r)-4-((5-fluoro-4-(3-(2-oxo-1,3-oxazinan-3-yl)phenyl)pyrimidin-2-yl)amino)cyclohexyl)-[1,4′-bipiperidine]-4-carboxamide (Compound 525). To a solution of 1′-(4-((2,6-dioxopiperidin-3-yl)amino)-2-fluorophenyl)-3′-fluoro-[1,4′-bipiperidine]-4-carboxylic acid (260 mg, 577 μmol, 1.00 eq) and trans-3-(3-(2-((4-aminocyclohexyl)amino)-5-fluoropyrimidin-4-yl)phenyl)-1,3-oxazinan-2-one (202 mg, 404 μmol, 0.700 eq, TFA) in DMF (5 mL) were added HATU (76.8 mg, 202 μmol, 0.350 eq) and DIEA (373 mg, 2.89 mmol, 503 μL, 5.00 eq) at 25 °C, the mixture was stirred at 25 °C for 0.5 h. LCMS showed 1′-(4-((2,6-dioxopiperidin-3-yl)amino)-2-fluorophenyl)-3′-fluoro-[1,4′-bipiperidine]-4-carboxylic acid was consumed completely and the desired target MS was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 10%-45% B over 8.0 min) to give the title compound (Compound 525) (53.8 mg, 65.1 μmol, 11.3% yield, 99.0% purity) as a pale yellow solid. LCMS (ES+): m/z 818.4 [M+H]⁺.

### Example 481. Synthesis of trans-1′-(4-((2,6-dioxopiperidin-3-yl)amino)-2-fluorophenyl)-3′,3′-difluoro-N-(4-((5-fluoro-4-(3-(2-oxo-1,3-oxazinan-3-yl)phenyl)pyrimidin-2-yl)amino)cyclohexyl)-[1,4′-bipiperidine]-4-carboxamide (Compound 500)

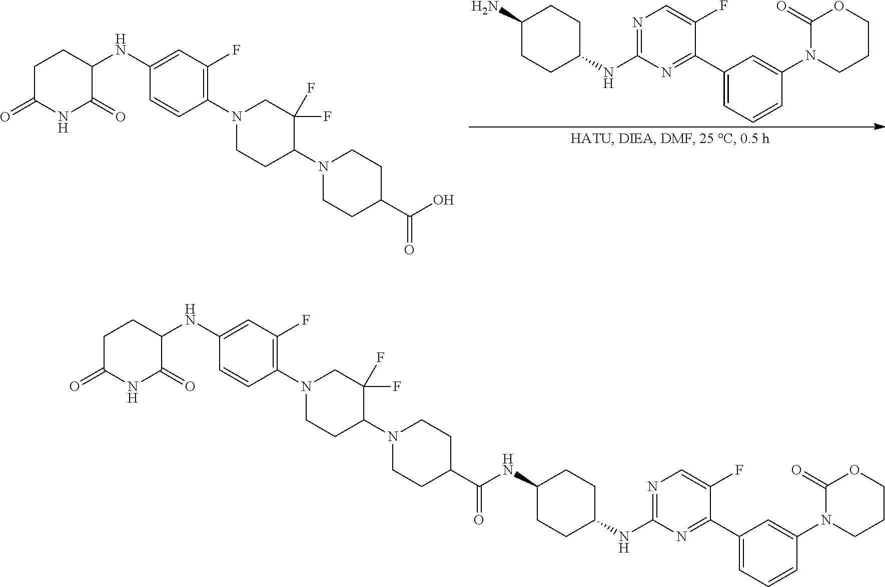 [SMILES: O=C1CCC(Nc2ccc(N3CCC(N4CCC(C(=O)O)CC4)C(F)(F)C3)c(F)c2)C(=O)N1 ; N[C@H]1CC[C@H](Nc2ncc(F)c(-c3cccc(N4CCCOC4=O)c3)n2)CC1 ; O=C1CCC(Nc2ccc(N3CCC(N4CCC(C(=O)N[C@H]5CC[C@H](Nc6ncc(F)c(-c7cccc(N8CCCOC8=O)c7)n6)CC5)CC4)C(F)(F)C3)c(F)c2)C(=O)N1]

Synthesis of trans-1′-(4-((2,6-dioxopiperidin-3-yl)amino)-2-fluorophenyl)-3′,3′ difluoro-N-(4-((5-fluoro-4-(3-(2-oxo-1,3-oxazinan-3-yl)phenyl)pyrimidin-2-yl)amino)cyclohexyl)-[1,4′-bipiperidine]-4-carboxamide (Compound 500). To a solution of 1′-(4-((2,6-dioxopiperidin-3-yl)amino)-2-fluorophenyl)-3′,3′-difluoro-[1,4′-bipiperidine]-4-carboxylic acid (200 mg, 427 μmol, 1.00 eq) in DMF (7 mL) were added HATU (64.9 mg, 171 μmol, 0.400 eq), DIEA (276 mg, 2.13 mmol, 372 μL, 5.00 eq) and trans-3-[3-[2-[(4-aminocyclohexyl)amino]-5-fluoro-pyrimidin-4-yl]phenyl]-1,3-oxazinan-2-one (149 mg, 299 μmol, 0.700 eq, TFA) at 25 °C. The mixture was stirred at 25 °C for 0.5 h. LCMS showed 1′-(4-((2,6-dioxopiperidin-3-yl)amino)-2-fluorophenyl)-3,3′-difluoro-[1,4′-bipiperidine]-4-carboxylic acid was consumed completely and the desired mass was detected. The reaction mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 40%-70% B over 8.0 min) to give the title compound (Compound 500) (61.6 mg, 68.8 μmol, 16.1% yield, 93.3% purity) as a pale yellow solid. LCMS (ES⁺): m/z 836.4 [M+H]⁺.

### Example 482. Synthesis of trans-3-((3-fluoro-4-(4-((1-(-4-((5-fluoro-4-(3-(2-oxo-1,3-oxazinan-3-yl)phenyl)pyrimidin-2-yl)amino)cyclohexane-1-carbonyl)piperidin-4-yl)methyl)piperidin-1-yl)phenyl)amino)piperidine-2,6-dione (Compound 425)

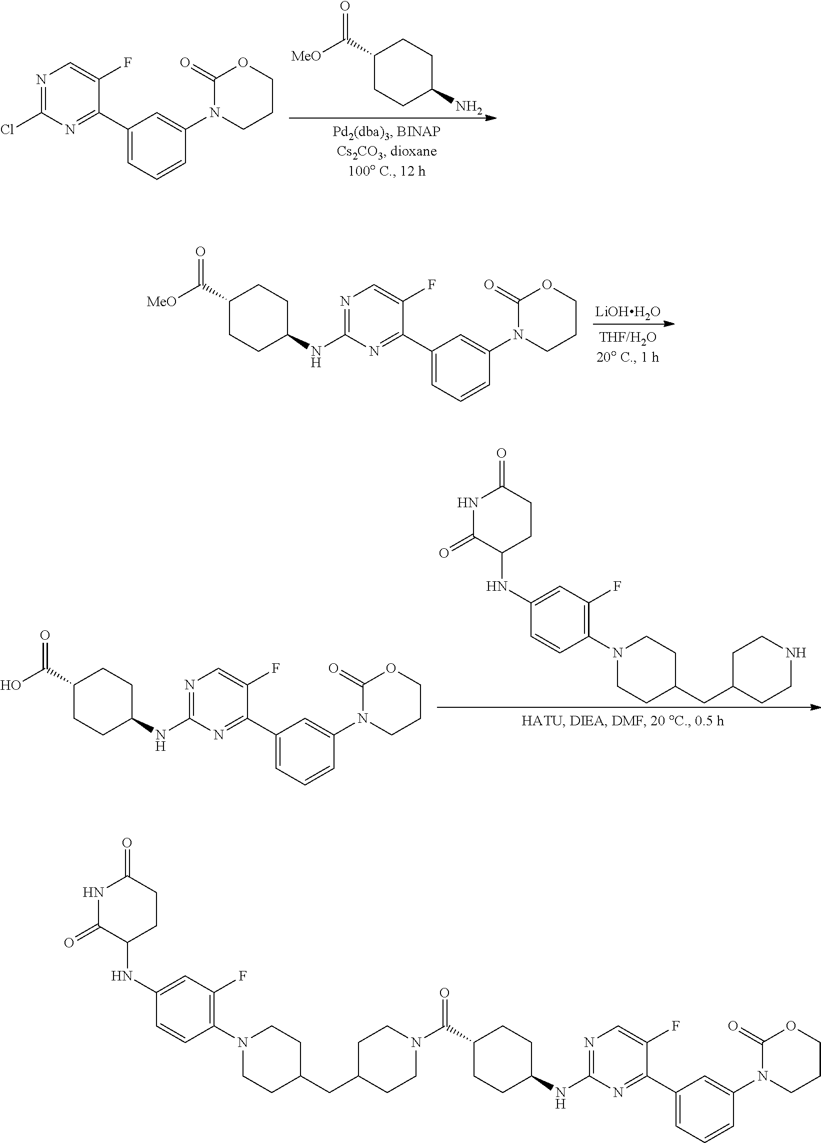

Synthesis of trans-methyl 4-((5-fluoro-4-(3-(2-oxo-1,3-oxazinan-3-yl)phenyl)pyrimidin-2-yl)amino)cyclohexane-1-carboxylate. To a solution of 3-(3-(2-chloro-5-fluoropyrimidin-4-yl)phenyl)-1,3-oxazinan-2-one (5.00 g, 16.3 mmol, 1.00 eq) and trans-methyl 4-aminocyclohexane-1-carboxylate (5.11 g, 32.5 mmol, 2.00 eq) in dioxane (100 mL) were added Pd₂(dba)₃ (149 mg, 162 μmol, 0.0100 eq), BINAP (101 mg, 162 μmol, 0.0100 eq) and Cs₂CO₃ (2.65 g, 8.12 mmol, 0.500 eq) at 25 °C under N₂, the mixture was stirred at 100 °C for 12 h under N₂. LCMS showed 3-(3-(2-chloro-5-fluoropyrimidin-4-yl)phenyl)-1,3-oxazinan-2-one was consumed completely and the desired mass was detected. The mixture was diluted with H₂O (100 mL) and then extracted with EtOAc (100 mL*3). The combined organic layers were dried over Na₂SO₄, filtered and the filtrate was concentrated under reduced pressure to give a residue. The residue was purified by MPLC (SiO₂, PE/EtOAc=1/0 to 3/1) to give the title compound (5.00 g, 11.7 mmol, 71.8% yield) as a yellow solid. LCMS (ES⁺): m/z 429.1 [M+H]⁺

Synthesis of trans-4-((5-fluoro-4-(3-(2-oxo-1,3-oxazinan-3-yl)phenyl)pyrimidin-2-yl)amino)cyclohexane-1-carboxylic acid. To a solution of trans-methyl 4-((5-fluoro-4-(3-(2-oxo-1,3-oxazinan-3-yl)phenyl)pyrimidin-2-yl)amino)cyclohexane-1-carboxylate (2.50 mg, 5.83 mmol, 1.00 eq) in THE (18 mL) and H₂O (9 mL) was added LiOH·H₂O (490 mg, 11.7 mmol, 2.00 eq) at 20 °C, the mixture was stirred at 20 °C for 1 h. LCMS showed trans-methyl 4-((5-fluoro-4-(3-(2-oxo-1,3-oxazinan-3-yl)phenyl)pyrimidin-2-yl)amino)cyclohexane-1-carboxylate was consumed completely and the desired mass was detected. The mixture was concentrated under reduced to remove THF. Then the remaining aqueous layer was acidified by HCl (1 M) to adjust pH to 3-4 and extracted with EtOAc (6 mL*3). The combined organic layers were dried over Na₂SO₄, filtered and the filtrate was concentrated under reduced pressure to give the title compound (480 mg, crude) as a yellow solid. LCMS (ES⁺): m/z 415.1 [M+H]⁺

Synthesis of trans-3-((3-fluoro-4-(4-((1-(-4-((5-fluoro-4-(3-(2-oxo-1,3-oxazinan-3-yl)phenyl)pyrimidin-2-yl)amino)cyclohexane-1-carbonyl)piperidin-4-yl)methyl)piperidin-1-yl)phenyl)amino)piperidine-2,6-dione (Compound 425). To a solution of trans-4-((5-fluoro-4-(3-(2-oxo-1,3-oxazinan-3-yl)phenyl)pyrimidin-2-yl)amino)cyclohexane-1-carboxylic acid (401 mg, 968 μmol, 0.500 eq) in DMF (8 mL) were added HATU (294 mg, 774 μmol, 0.400 eq) and DIEA (1.25 g, 9.68 mmol, 1.69 mL, 5.00 eq) at 20 °C. Then 3-((3-fluoro-4-(4-(piperidin-4-ylmethyl)piperidin-1-yl)phenyl)amino)piperidine-2,6-dione (1.00 g, 1.94 mmol, 1.00 eq, TFA) was added to the mixture, the mixture was stirred at 20 °C for 0.5 h. LCMS showed 3-((3-fluoro-4-(4-(piperidin-4-ylmethyl)piperidin-1-yl)phenyl)amino)piperidine-2,6-dione was consumed and the desired target MS was detected. The mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 15%-50% B over 8.0 min) to give the title compound (Compound 425) (219 mg, 257 μmol, 13.3% yield, 94.0% purity) as a gray solid. LCMS (ES⁺): m/z 799.4 [M+H]⁺

### Example 483. Synthesis of trans-3-[3-fluoro-4-[4-[[1-[4-[[5-fluoro-4-[3-(2-oxo-1,3-oxazinan-3-yl)phenyl]pyrimidin-2-yl]amino]cyclohexanecarbonyl]-4-piperidyl]methyl]piperazin-1-yl]anilino]piperidine-2,6-dione (Compound 426)

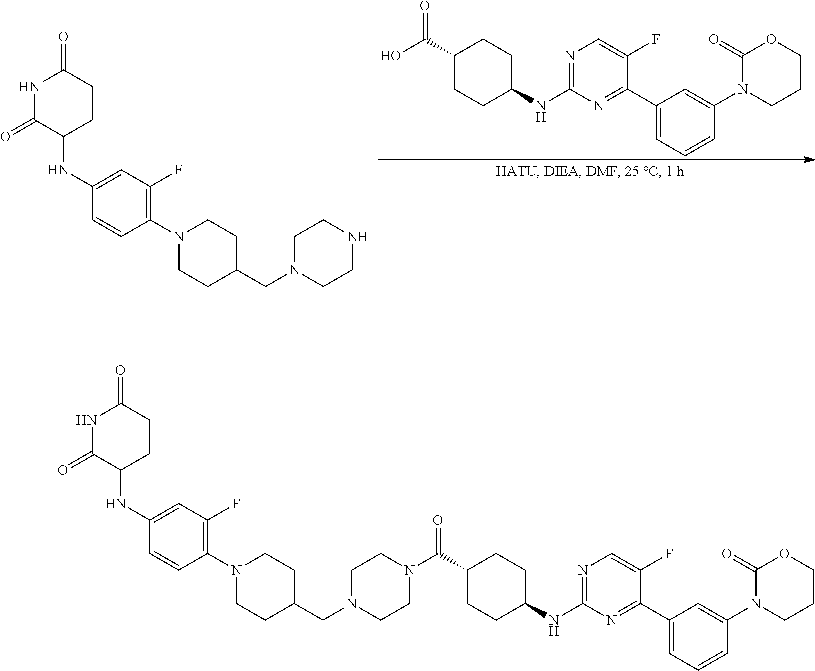 [SMILES: O=C1CCC(Nc2ccc(N3CCC(CN4CCNCC4)CC3)c(F)c2)C(=O)N1 ; O=C1OCCCN1c1cccc(-c2nc(N[C@H]3CC[C@H](C(=O)O)CC3)ncc2F)c1 ; O=C1CCC(Nc2ccc(N3CCC(CN4CCN(C(=O)[C@H]5CC[C@H](Nc6ncc(F)c(-c7cccc(N8CCCOC8=O)c7)n6)CC5)CC4)CC3)c(F)c2)C(=O)N1]

Synthesis of trans-3-[3-fluoro-4-[4-[[1-[4-[[5-fluoro-4-[3-(2-oxo-1,3-oxazinan-3-yl)phenyl]pyrimidin-2-yl]amino]cyclohexanecarbonyl]-4-piperidyl]methyl]piperazin-1-yl]anilino]piperidine-2,6-dione (Compound 426). To a solution of trans-4-((5-fluoro-4-(3-(2-oxo-1,3-oxazinan-3-yl)phenyl)pyrimidin-2-yl)amino)cyclohexane-1-carboxylic acid (320 mg, 773 μmol, 1.00 eq) in DMF (5 mL) were added HATU (441 mg, 1.16 mmol, 1.50 eq) and DIEA (300 mg, 2.32 mmol, 404 μL, 3.00 eq) at 25 °C. Then 3-((3-fluoro-4-(4-(piperidin-4-ylmethyl)piperazin-1-yl)phenyl)amino)piperidine-2,6-dione (400 mg, 773 μmol, 1.00 eq, TFA) was added at 25 °C. The resulting mixture was stirred at 25 °C for 1 h. LCMS showed that 3-((3-fluoro-4-(4-(piperidin-4-ylmethyl)piperazin-1-yl)phenyl)amino)piperidine-2,6-dione was consumed completely and the desired mass was detected. The mixture was filtered and filtrate was concentrate under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 30%-55% B over 8.0 min) to give the title compound (Compound 426) (116 mg, 141 μmol, 18.2% yield, 97.4% purity) as a white solid. LCMS (ES⁺): m/z 800.4 [M+H]⁺

### Example 484. Synthesis of trans-3-((3-fluoro-4-(1-((1-(4-((5-fluoro-4-(3-(2-oxo-1,3-oxazinan-3-yl)phenyl)pyrimidin-2-yl)amino)cyclohexane-1-carbonyl)piperidin-4-yl)methyl)piperidin-4-yl)phenyl)amino)piperidine-2,6-dione (Compound 427)

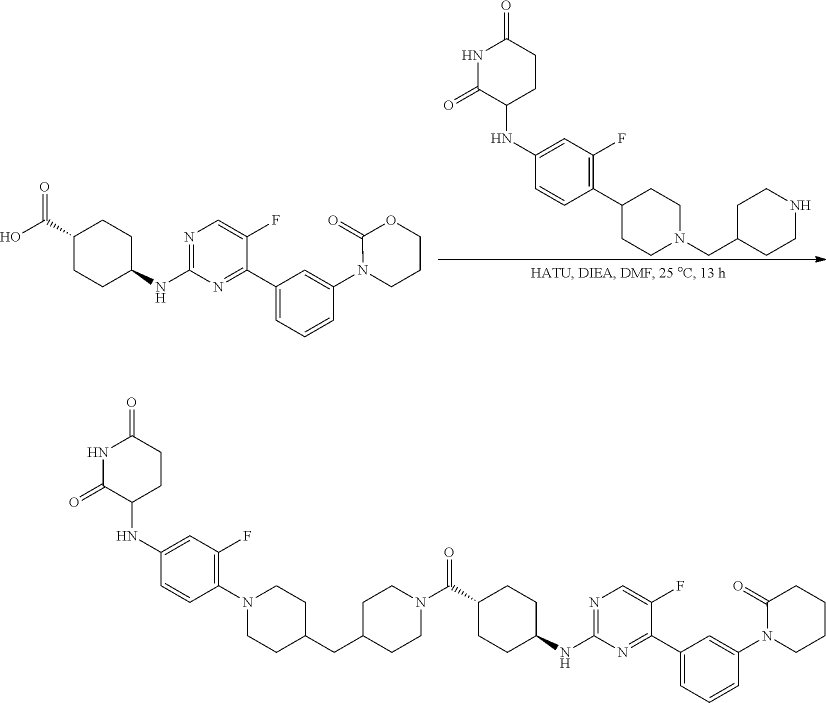 [SMILES: O=C1OCCCN1c1cccc(-c2nc(N[C@H]3CC[C@H](C(=O)O)CC3)ncc2F)c1 ; O=C1CCC(Nc2ccc(C3CCN(CC4CCNCC4)CC3)c(F)c2)C(=O)N1 ; O=C1CCC(Nc2ccc(N3CCC(CC4CCN(C(=O)[C@H]5CC[C@H](Nc6ncc(F)c(-c7cccc(N8CCCCC8=O)c7)n6)CC5)CC4)CC3)c(F)c2)C(=O)N1]

Synthesis of trans-3-((3-fluoro-4-(1-((1-(4-((5-fluoro-4-(3-(2-oxo-1,3-oxazinan-3-yl)phenyl)pyrimidin-2-yl)amino)cyclohexane-1-carbonyl)piperidin-4-yl)methyl)piperidin-4-yl)phenyl)amino)piperidine-2,6-dione (Compound 427). To a solution of trans-4-((5-fluoro-4-(3-(2-oxo-1,3-oxazinan-3-yl)phenyl)pyrimidin-2-yl)amino)cyclohexane-1-carboxylic acid (480 mg, 1.16 mmol, 1.00 eq) in DMF (10 mL) were added HATU (661 mg, 1.74 mmol, 1.50 eq) and DIEA (1.50 g, 5.80 mmol, 1.01 mL, 5.00 eq) at 25 °C, then to the mixture was added 3-((3-fluoro-4-(1-(piperidin-4-ylmethyl)piperidin-4-yl)phenyl)amino)piperidine-2,6-dione (778 mg, 1.51 mmol, 1.30 eq, TFA) at 25 °C. The resulting mixture was stirred at 25 °C for 13 h. LCMS showed trans-4-((5-fluoro-4-(3-(2-oxo-1,3-oxazinan-3-yl)phenyl)pyrimidin-2-yl)amino)cyclohexane-1-carboxylic acid was consumed completely and the desired mass was detected. The mixture was concentrated under reduced pressure to give a residue. The residue was purified by prep-HPLC (column: Waters Xbridge Prep OBD C18 150*40 mm*10 um; mobile phase: [H₂O (0.05% NH₃H₂O+10 mM NH₄HCO₃)-ACN]; gradient: 30%-60% B over 8.0 min) to give the title compound (Compound 427) (247 mg, 307 μmol, 26.5% yield, 99.4% purity) as a white solid. LCMS (ES⁺): m/z 799.4 [M+H]⁺

### Example 485. Synthesis of trans-(R)-3-((3-fluoro-4-(1-((1-((1r,4r)-4-((5-fluoro-4-(3-(2-oxo-1,3-oxazinan-3-yl)phenyl)pyrimidin-2-yl)amino)cyclohexane-1-carbonyl)piperidin-4-yl)methyl)piperidin-4-yl)phenyl)amino)piperidine-2,6-dione (Compound 601) and trans-(S)-3-((3-fluoro-4-(1-((1-((1r,4r)-4-((5-fluoro-4-(3-(2-oxo-1,3-oxazinan-3-yl)phenyl)pyrimidin-2-yl)amino)cyclohexane-1-carbonyl)piperidin-4-yl)methyl)piperidin-4-yl)phenyl)amino)piperidine-2,6-dione (Compound 600) (The Two Isomers were Assigned Arbitrarily)

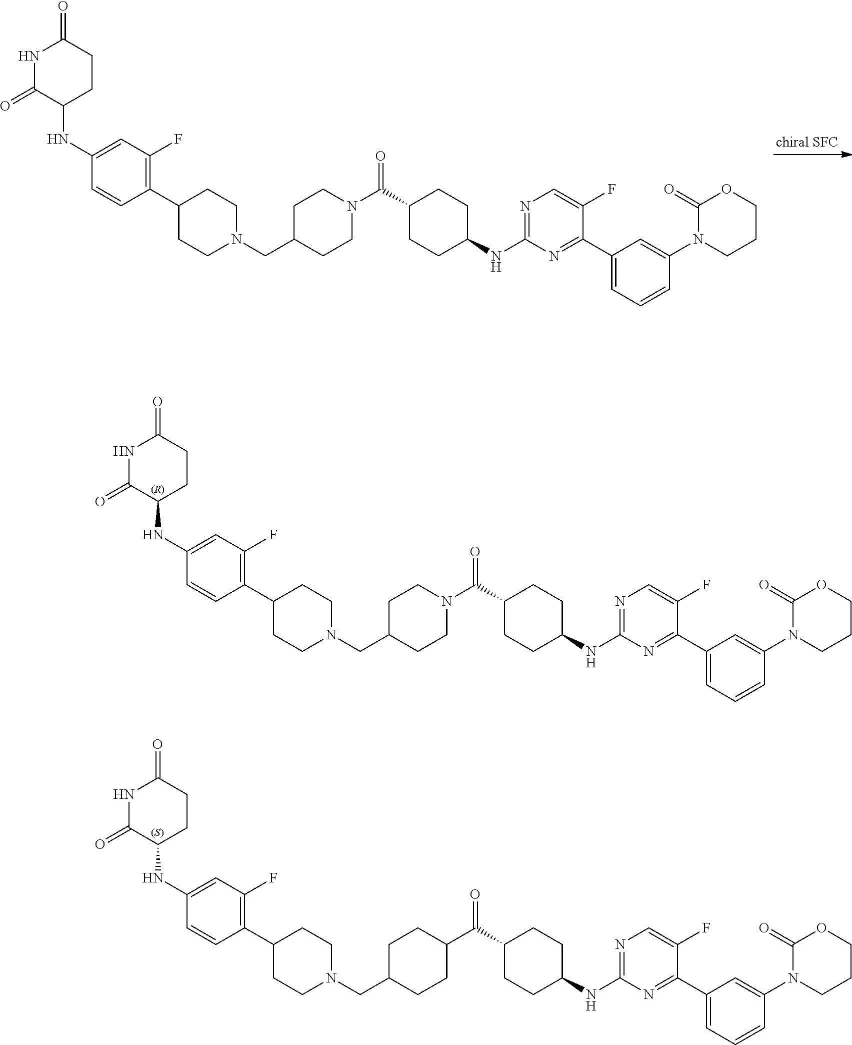

Synthesis of trans-(R)-3-((3-fluoro-4-(1-((1-((1r,4r)-4-((5-fluoro-4-(3-(2-oxo-1,3-oxazinan-3-yl)phenyl)pyrimidin-2-yl)amino)cyclohexane-1-carbonyl)piperidin-4-yl)methyl)piperidin-4-yl)phenyl)amino)piperidine-2,6-dione (Compound 601) and trans-(S)-3-((3-fluoro-4-(1-((1-((1r,4r)-4-((5-fluoro-4-(3-(2-oxo-1,3-oxazinan-3-yl)phenyl)pyrimidin-2-yl)amino)cyclohexane-1-carbonyl)piperidin-4-yl)methyl)piperidin-4-yl)phenyl)amino)piperidine-2,6-dione (Compound 600) (the two isomers were assigned arbitrarily). Trans-3-((3-fluoro-4-(1-((1-((1r,4r)-4-((5-fluoro-4-(3-(2-oxo-1,3-oxazinan-3-yl)phenyl)pyrimidin-2-yl)amino)cyclohexane-1-carbonyl)piperidin-4-yl)methyl)piperidin-4-yl)phenyl)amino)piperidine-2,6-dione (Compound 427) (200 mg, 250 μmol, 1.00 eq) was separated by chiral SFC (column: ChiralPak IH, 250*30 mm, 10 um; mobile phase: [CO₂-IPA (0.1% IPAm)]; B %: 62%, isocratic elution mode) to give the title compound (Compound 601) (83.6 mg, 104 μmol, 41.6% yield, 99.4% purity, 98.9% ee) (retention time: 1.397, peak 1) as a white solid. LCMS (ES⁺): m/z 799.4 [M+H]⁺ and title compound (Compound 600) (104 mg, 128 μmol, 51.2% yield, 98.8% purity, 99.4% ee) (retention time: 2.568, peak 2) as a white solid. LCMS (ES⁺): m/z 799.4 [M+H]⁺

### Example 486. Synthesis of trans-3-((3-fluoro-4-(4-((4-(4-((5-fluoro-4-(3-(2-oxo-1,3-oxazinan-3-yl)phenyl)pyrimidin-2-yl)amino)cyclohexane-1-carbonyl)piperazin-1-yl)methyl)piperidin-1-yl)phenyl)amino)piperidine-2,6-dione (Compound 428)

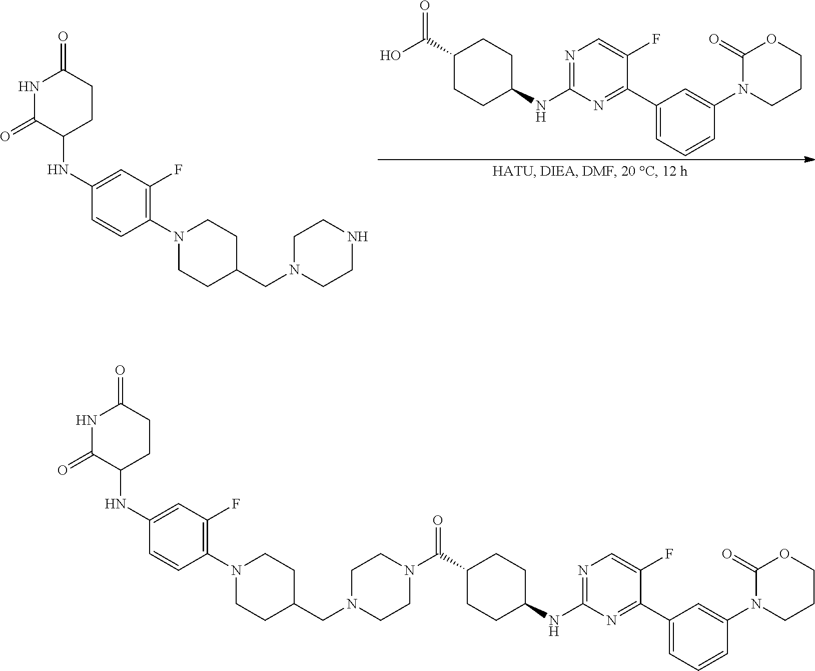 [SMILES: O=C1CCC(Nc2ccc(N3CCC(CN4CCNCC4)CC3)c(F)c2)C(=O)N1 ; O=C1OCCCN1c1cccc(-c2nc(N[C@H]3CC[C@H](C(=O)O)CC3)ncc2F)c1 ; O=C1CCC(Nc2ccc(N3CCC(CN4CCN(C(=O)[C@H]5CC[C@H](Nc6ncc(F)c(-c7cccc(N8CCCOC8=O)c7)n6)CC5)CC4)CC3)c(F)c2)C(=O)N1]

Synthesis of trans-3-((3-fluoro-4-(4-((4-(4-((5-fluoro-4-(3-(2-oxo-1,3-oxazinan-3-yl)phenyl)pyrimidin-2-yl)amino)cyclohexane-1-carbonyl)piperazin-1-yl)methyl)piperidin-1-yl)phenyl)amino)piperidine-2,6-dione (Compound 428). To a solution of trans-4-((5-fluoro-4-(3-(2-oxo-1,3-oxazinan-3-yl)phenyl)pyrimidin-2-yl)amino)cyclohexane-1-carboxylic acid (500 mg, 1.21 mmol, 1.00 eq) in DMF (8 mL) were added DIEA (312 mg, 2.41 mmol, 420 L, 2.00 eq) and HATU (550 mg, 1.45 mmol, 1.20 eq) at 20 °C. Then to the mixture was added 3-((3-fluoro-4-(4-(piperazin-1-ylmethyl)piperidin-1-yl)phenyl)amino)piperidine-2,6-dione (624 mg, 1.21 mmol, 1.00 eq, TFA) at 20 °C. The mixture was stirred at 20 °C for 12 h. LCMS showed 3-((3-fluoro-4-(4-(piperazin-1-ylmethyl)piperidin-1-yl)phenyl)amino)piperidine-2,6-dione was consumed completely and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min) to give the title compound (Compound 428) (268 mg, 328 μmol, 27.2% yield, 98.0% purity) as a pink solid. LCMS (ES⁺): m/z 800.4 [M+H]⁺

### Example 487. Synthesis of trans-N-(trans-4-(4-(4-((2,6-dioxopiperidin-3-yl)amino)-2-fluorophenyl)piperidin-1-yl)cyclohexyl)-4-((5-fluoro-4-(3-(2-oxo-1,3-oxazinan-3-yl)phenyl)pyrimidin-2-yl)amino)cyclohexane-1-carboxamide (Compound 563

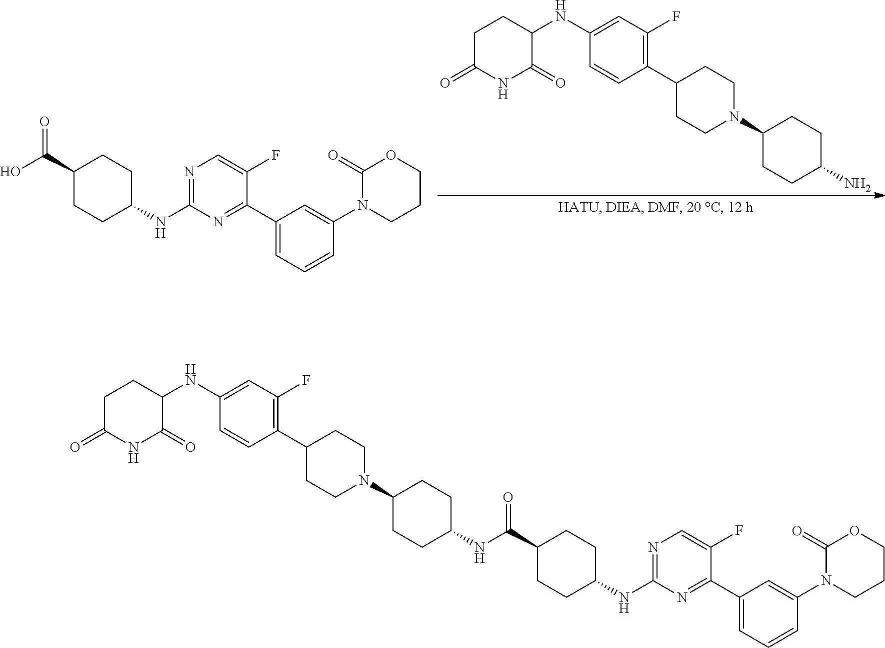 [SMILES: O=C1OCCCN1c1cccc(-c2nc(N[C@H]3CC[C@H](C(=O)O)CC3)ncc2F)c1 ; N[C@H]1CC[C@H](N2CCC(c3ccc(NC4CCC(=O)NC4=O)cc3F)CC2)CC1 ; O=C1CCC(Nc2ccc(C3CCN([C@H]4CC[C@H](NC(=O)[C@H]5CC[C@H](Nc6ncc(F)c(-c7cccc(N8CCCOC8=O)c7)n6)CC5)CC4)CC3)c(F)c2)C(=O)N1]

Synthesis of trans-N-(trans-4-(4-(4-((2,6-dioxopiperidin-3-yl)amino)-2-fluorophenyl)piperidin-1-yl)cyclohexyl)-4-((5-fluoro-4-(3-(2-oxo-1,3-oxazinan-3-yl)phenyl)pyrimidin-2-yl)amino)cyclohexane-1-carboxamide (Compound 563). To a solution of trans-(1r,4r)-4-((5-fluoro-4-(3-(2-oxo-1,3-oxazinan-3-yl)phenyl)pyrimidin-2-yl)amino)cyclohexane-1-carboxylic acid (201 mg, 484 μmol, 1.00 eq) and trans-3-((4-(1-((1r,4r)-4-aminocyclohexyl)piperidin-4-yl)-3-fluorophenyl)amino)piperidine-2,6-dione (250 mg, 484 μmol, 1.00 eq, TFA) in DMF (5 mL) were added HATU (460 mg, 1.21 mmol, 2.50 eq) and DIEA (188 mg, 1.45 mmol, 253 μL, 3.00 eq) at 20 °C. The mixture was stirred at 20 °C for 12 h. LC-MS showed that trans-(1r,4r)-4-((5-fluoro-4-(3-(2-oxo-1,3-oxazinan-3-yl)phenyl)pyrimidin-2-yl)amino)cyclohexane-1-carboxylic acid was consumed completely and desired mass was detected. The reaction mixture was diluted with H₂O (10 mL) and extracted with EtOAc (5 mL*5). The combined organic layers were washed with brine (10 mL), dried over anhydrous Na₂SO₄, filtered and concentrated under reduced pressure. The residue was purified by prep-HPLC (column: 3_Phenomenex Luna C18 75*30 mm*3 um; mobile phase: [H₂O (0.2% FA)-ACN]; gradient: 5%-35% B over 8.0 min) to give the title compound (Compound 563) (85.1 mg, 104 μmol, 21.4% yield, 97.4% purity) as a yellow solid. LCMS (ES⁺): m/z 799.3 [M+H]⁺

### Example 488. Synthesis of trans-N-(trans-4-(4-(4-((2,6-dioxopiperidin-3-yl)amino)-2-fluorophenyl)piperazin-1-yl)cyclohexyl)-4-((5-fluoro-4-(3-(2-oxo-1,3-oxazinan-3-yl)phenyl)pyrimidin-2-yl)amino)cyclohexane-1-carboxamide (Compound 490)

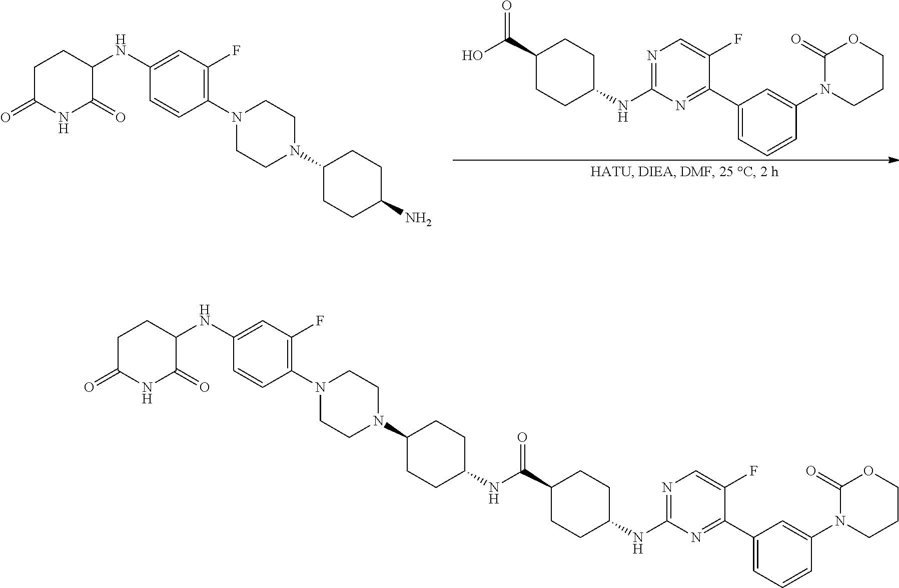 [SMILES: N[C@H]1CC[C@H](N2CCN(c3ccc(NC4CCC(=O)NC4=O)cc3F)CC2)CC1 ; O=C1OCCCN1c1cccc(-c2nc(N[C@H]3CC[C@H](C(=O)O)CC3)ncc2F)c1 ; O=C1CCC(Nc2ccc(N3CCN([C@H]4CC[C@H](NC(=O)[C@H]5CC[C@H](Nc6ncc(F)c(-c7cccc(N8CCCOC8=O)c7)n6)CC5)CC4)CC3)c(F)c2)C(=O)N1]

Synthesis of trans-N-(trans-4-(4-(4-((2,6-dioxopiperidin-3-yl)amino)-2-fluorophenyl)piperazin-1-yl)cyclohexyl)-4-((5-fluoro-4-(3-(2-oxo-1,3-oxazinan-3-yl)phenyl)pyrimidin-2-yl)amino)cyclohexane-1-carboxamide (Compound 490). To a solution of rac-3-((4-(4-((1r,4r)-4-aminocyclohexyl)piperazin-1-yl)-3-fluorophenyl)amino)piperidine-2,6-dione (300 mg, 580 μmol, 1.00 eq, TFA) in DMF (2 mL) was added DIEA (225 mg, 1.74 mmol, 303 μL, 3.00 eq), trans-(1r,4r)-4-((5-fluoro-4-(3-(2-oxo-1,3-oxazinan-3-yl)phenyl)pyrimidin-2-yl)amino)cyclohexane-1-carboxylic acid (96.1 mg, 232 μmol, 0.400 eq) and HATU (331 mg, 870 μmol, 1.50 eq) at 25 °C. The mixture was stirred at 25 °C for 2 h. LCMS showed that trans-3-((4-(4-(4-aminocyclohexyl)piperazin-1-yl)-3-fluorophenyl)amino)piperidine-2,6-dione was consumed completely, and the desired mass was detected. The mixture was concentrated under reduced pressure. The residue was purified by prep-HPLC (column: Waters Xbridge BEH C18 100*30 mm*10 um; mobile phase: [H₂O (10 mM NH₄HCO₃)-ACN]; gradient: 29%-69% B over 8.0 min) to give title (Compound 490) (38.9 mg, 48.3 μmol, 8.34% yield, 99.4% purity) as a brown solid. LCMS (ES⁺): m/z 800.4 [M+H]⁺.

### Example 489. Synthesis of trans-3-((3-fluoro-4-(4-((1-(4-((5-fluoro-4-(3-(2-oxo-1,3-oxazinan-3-yl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 583)

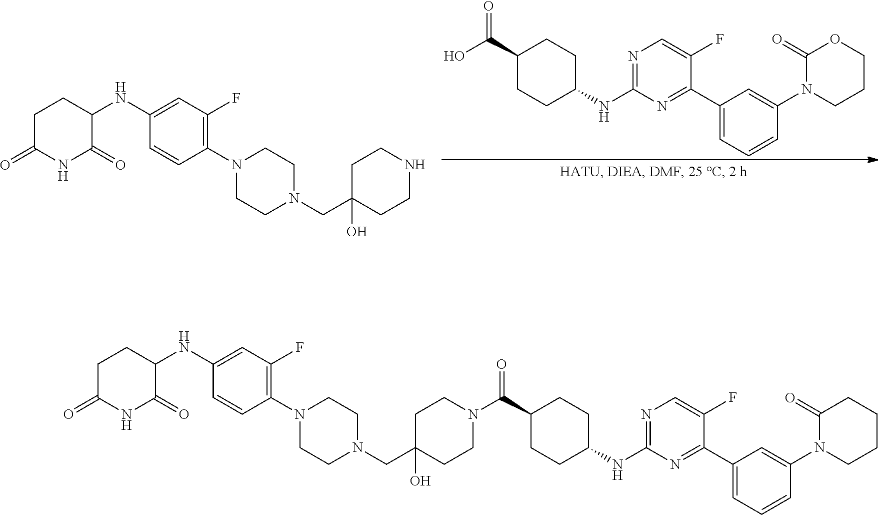 [SMILES: O=C1CCC(Nc2ccc(N3CCN(CC4(O)CCNCC4)CC3)c(F)c2)C(=O)N1 ; O=C1OCCCN1c1cccc(-c2nc(N[C@H]3CC[C@H](C(=O)O)CC3)ncc2F)c1 ; O=C1CCC(Nc2ccc(N3CCN(CC4(O)CCN(C(=O)[C@H]5CC[C@H](Nc6ncc(F)c(-c7cccc(N8CCCCC8=O)c7)n6)CC5)CC4)CC3)c(F)c2)C(=O)N1]

Synthesis of trans-3-((3-fluoro-4-(4-((1-(4-((5-fluoro-4-(3-(2-oxo-1,3-oxazinan-3-yl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 583). To a solution of 3-((3-fluoro-4-(4-((4-hydroxypiperidin-4-yl)methyl)piperazin-1-yl)phenyl)amino)piperidine-2,6-dione (500 mg, 1.19 mmol, 1.00 eq) in DMF (5 mL) was added HATU (453 mg, 1.19 mmol, 1.00 eq), trans-4-[[5-fluoro-4-[3-(2-oxo-1,3-oxazinan-3-yl)phenyl]pyrimidin-2-yl]amino]cyclohexanecarboxylic acid (493 mg, 1.19 mmol, 1.00 eq) and DIEA (462 mg, 3.58 mmol, 622 μL, 3.00 eq) at 25 °C. The mixture was stirred at 25 °C for 2 h. LC-MS showed 3-((3-fluoro-4-(4-((4-hydroxypiperidin-4-yl)methyl)piperazin-1-yl)phenyl)amino)piperidine-2,6-dione was consumed completely and the desired mass was detected. The reaction mixture was concentrated under reduced pressure. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to give the title compound (Compound 583) (134 mg, 158 μmol, 13.3% yield, 96.0% purity) as a pale yellow solid. LCMS (ES⁺): m/z 816.4 [M+H]⁺.

### Example 490. Synthesis of trans-3-((3-fluoro-4-(1-((1-(4-((5-fluoro-4-(3-(2-oxo-1,3-oxazinan-3-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 580)

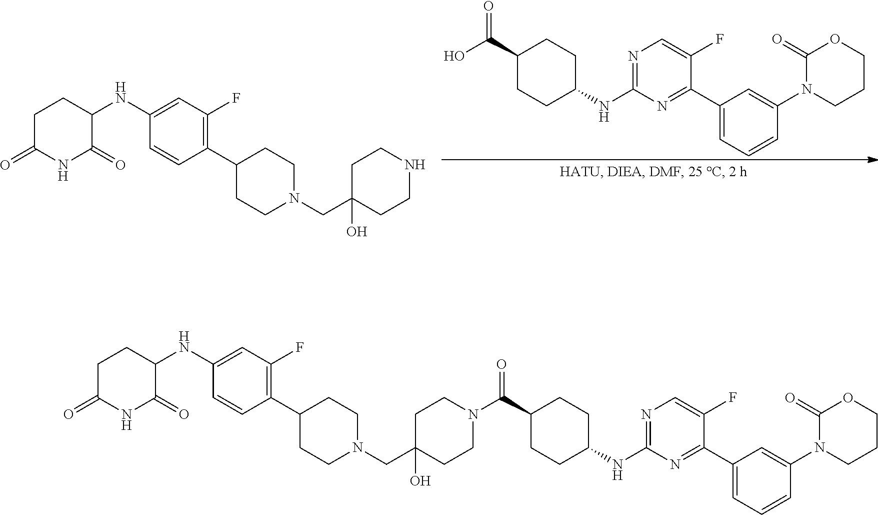 [SMILES: O=C1CCC(Nc2ccc(C3CCN(CC4(O)CCNCC4)CC3)c(F)c2)C(=O)N1 ; O=C1OCCCN1c1cccc(-c2nc(N[C@H]3CC[C@H](C(=O)O)CC3)ncc2F)c1 ; O=C1CCC(Nc2ccc(C3CCN(CC4(O)CCN(C(=O)[C@H]5CC[C@H](Nc6ncc(F)c(-c7cccc(N8CCCOC8=O)c7)n6)CC5)CC4)CC3)c(F)c2)C(=O)N1]

Synthesis of trans-3-((3-fluoro-4-(1-((1-(4-((5-fluoro-4-(3-(2-oxo-1,3-oxazinan-3-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 580). To a solution of 3-((3-fluoro-4-(1-((4-hydroxypiperidin-4-yl)methyl)piperidin-4-yl)phenyl)amino)piperidine-2,6-dione (500 mg, 938 μmol, 1.00 eq, TFA) in DMF (5 mL) was added trans-4-((5-fluoro-4-(3-(2-oxo-1,3-oxazinan-3-yl)phenyl)pyrimidin-2-yl)amino)cyclohexane-1-carboxylic acid (233 mg, 563 μmol, 0.60 eq), DIEA (121 mg, 938 μmol, 163 μL, 1.00 eq) and HATU (357 mg, 938 μmol, 1.00 eq) at 25 °C. The mixture was stirred at 25 °C for 2 h. LC-MS showed the desired mass was detected. The reaction mixture was concentrated under reduced pressure. The residue was purified by prep-HPLC (column: Waters Xbridge Prep OBD C18 150*40 mm*10 um; mobile phase: [H₂O (10 mM NH₄HCO₃)-ACN]; gradient: 40%-70% B over 8.0 min) to give the title compound (Compound 580) (104 mg, 127 μmol, 13.6% yield, 99.6% purity) as an off-white solid. LCMS (ES⁺): m/z 815.4 [M+H]⁺.

### Example 491. Synthesis of trans-(1r,4R)—N-(((1r,4R)-4-((4-(4-((2,6-dioxopiperidin-3-yl)amino)-2-fluorophenyl)piperazin-1-yl)methyl)cyclohexyl)methyl)-4-((5-fluoro-4-(3-(2-oxo-1,3-oxazinan-3-yl)phenyl)pyrimidin-2-yl)amino)cyclohexane-1-carboxamide (Compound 591)

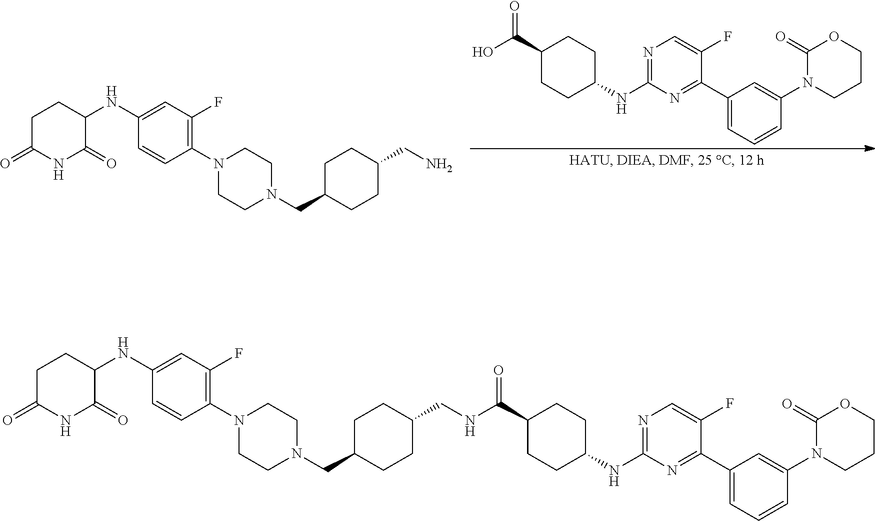 [SMILES: NC[C@H]1CC[C@H](CN2CCN(c3ccc(NC4CCC(=O)NC4=O)cc3F)CC2)CC1 ; O=C1OCCCN1c1cccc(-c2nc(N[C@H]3CC[C@H](C(=O)O)CC3)ncc2F)c1 ; O=C1CCC(Nc2ccc(N3CCN(C[C@H]4CC[C@H](CNC(=O)[C@H]5CC[C@H](Nc6ncc(F)c(-c7cccc(N8CCCOC8=O)c7)n6)CC5)CC4)CC3)c(F)c2)C(=O)N1]

Synthesis of trans-(1r,4R)—N-(((1r,4R)-4-((4-(4-((2,6-dioxopiperidin-3-yl)amino)-2-fluorophenyl)piperazin-1-yl)methyl)cyclohexyl)methyl)-4-((5-fluoro-4-(3-(2-oxo-1,3-oxazinan-3-yl)phenyl)pyrimidin-2-yl)amino)cyclohexane-1-carboxamide (Compound 591). To a solution of trans-4-[[5-fluoro-4-[3-(2-oxo-1,3-oxazinan-3-yl) phenyl]pyrimidin-2-yl]amino]cyclohexane carboxylic acid (176 mg, 425 μmol, 0.800 eq) in DMF (5 mL) were added HATU (303 mg, 797 μmol, 1.50 eq), DIEA (206 mg, 1.59 mmol, 278 μL, 3.00 eq) at 25 °C. Then trans-3-[4-[4-[[4-(aminomethyl) cyclohexyl]methyl]piperazin-1-yl]-3-fluoro-anilino]piperidine-2,6-dione (290 mg, 532 μmol, 1.00 eq, TFA) was added at 25 °C. The resulting mixture was stirred at 25 °C for 12 h. LCMS showed that trans-3-[4-[4-[[4-(aminomethyl) cyclohexyl]methyl]piperazin-1-yl]-3-fluoro-anilino]piperidine-2,6-dione was consumed completely and the desired mass was detected. The mixture was concentrated under reduced pressure to give a residue. The residue was purified by prep-HPLC (column: Phenomenex Gemini-NX 80*40 mm*3 um; mobile phase: [H₂O (10 mM NH₄HCO₄)-ACN]; gradient: 20%-50% B over 8.0 min) to give the title compound (Compound 591) (36.3 mg, 42.1 μmol, 7.91% yield, 95.9% purity) as a white solid. LCMS (ES⁺): m/z 828.4 [M+H]⁺.

### Example 492. Synthesis of trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(2-oxo-1,3-oxazinan-3-yl)phenyl)pyrimidin-2-yl)amino)cyclohexane-1-carboxamide (Compound 486)

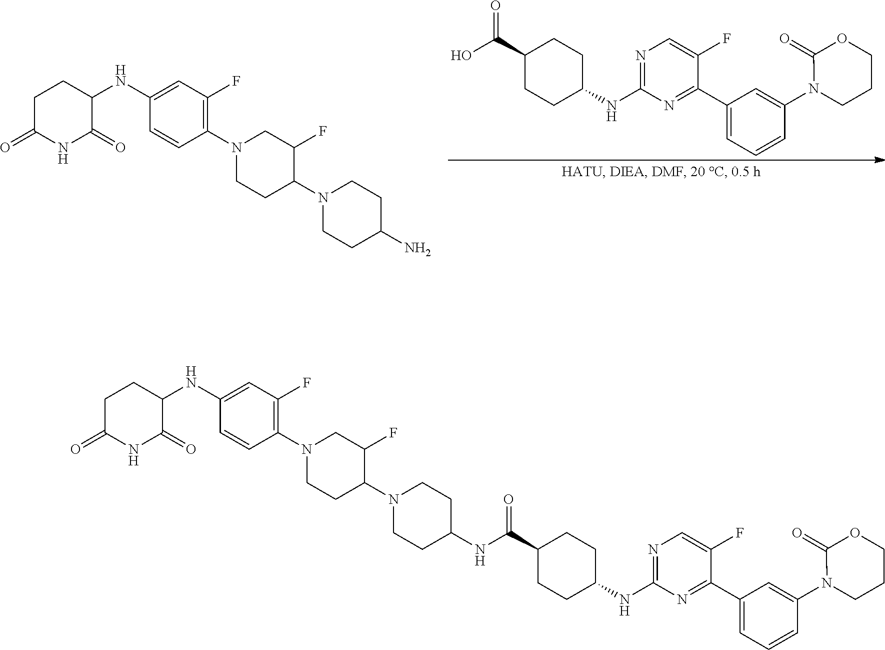 [SMILES: NC1CCN(C2CCN(c3ccc(NC4CCC(=O)NC4=O)cc3F)CC2F)CC1 ; O=C1OCCCN1c1cccc(-c2nc(N[C@H]3CC[C@H](C(=O)O)CC3)ncc2F)c1 ; O=C1CCC(Nc2ccc(N3CCC(N4CCC(NC(=O)[C@H]5CC[C@H](Nc6ncc(F)c(-c7cccc(N8CCCOC8=O)c7)n6)CC5)CC4)C(F)C3)c(F)c2)C(=O)N1]

Synthesis of trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(2-oxo-1,3-oxazinan-3-yl)phenyl)pyrimidin-2-yl)amino)cyclohexane-1-carboxamide (Compound 486). To a solution of trans-4-((5-fluoro-4-(3-(2-oxo-1,3-oxazinan-3-yl)phenyl)pyrimidin-2-yl)amino)cyclohexane-1-carboxylic acid (58.8 mg, 142 μmol, 0.400 eq) in DMF (4 mL) were added HATU (47.2 mg, 124 μmol, 0.350 eq) and DIEA (235 mg, 1.80 mmol, 315 μL, 5.00 eq) at 20 °C, then 3-((4-(4-amino-3′-fluoro-[1,4′-bipiperidin]-1′-yl)-3-fluorophenyl)amino)piperidine-2,6-dione (190 mg, 355 μmol, 1.00 eq, TFA) was added to the mixture at 20 °C, the mixture was stirred at 20 °C for 0.5 h. LCMS showed 3-((4-(4-amino-3′-fluoro-[1,4′-bipiperidin]-1′-yl)-3-fluorophenyl)amino)piperidine-2,6-dione was consumed completely and the desired target MS was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 10%-45% B over 8.0 min) to give the title compound (Compound 486) (12.4 mg, 14.8 μmol, 4.18% yield, 97.8% purity) as a brown solid. LCMS (ES+): m/z 818.5 [M+H]⁺

### Example 493. Synthesis of trans-N-(1′-(4-((2,6-dioxopiperidin-3-yl)amino)-2-fluorophenyl)-3′,3′-difluoro-[1,4′-bipiperidin]-4-yl)-4-((5-fluoro-4-(3-(2-oxo-1,3-oxazinan-3-yl)phenyl)pyrimidin-2-yl)amino)cyclohexane-1-carboxamide (Compound 495)

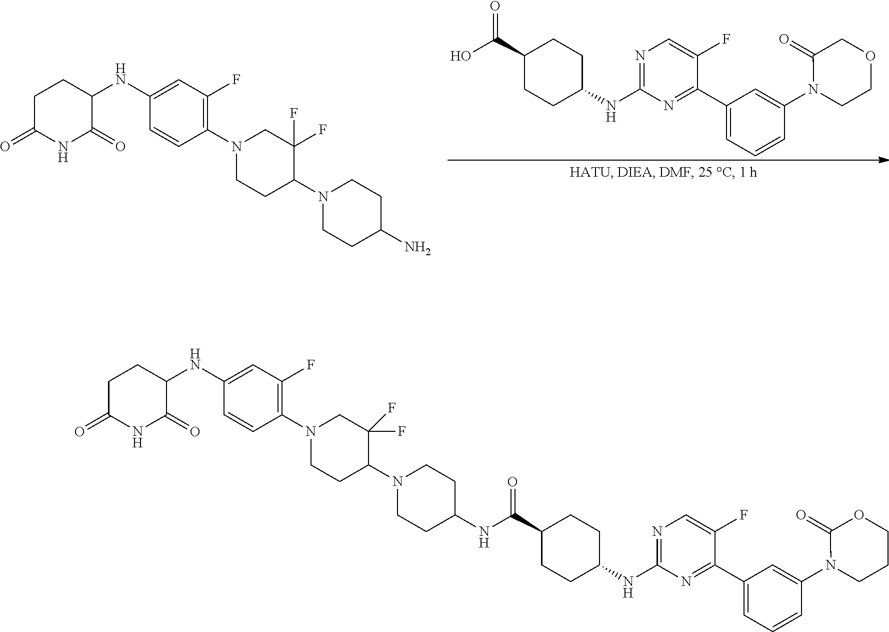 [SMILES: NC1CCN(C2CCN(c3ccc(NC4CCC(=O)NC4=O)cc3F)CC2(F)F)CC1 ; O=C1COCCN1c1cccc(-c2nc(N[C@H]3CC[C@H](C(=O)O)CC3)ncc2F)c1 ; O=C1CCC(Nc2ccc(N3CCC(N4CCC(NC(=O)[C@H]5CC[C@H](Nc6ncc(F)c(-c7cccc(N8CCCOC8=O)c7)n6)CC5)CC4)C(F)(F)C3)c(F)c2)C(=O)N1]

Synthesis of trans-N-(1′-(4-((2,6-dioxopiperidin-3-yl)amino)-2-fluorophenyl)-3,3′-difluoro-[1,4′-bipiperidin]-4-yl)-4-((5-fluoro-4-(3-(2-oxo-1,3-oxazinan-3-yl)phenyl)pyrimidin-2-yl)amino)cyclohexane-1-carboxamide (Compound 495). To a solution of trans-4-((5-fluoro-4-(3-(2-oxo-1,3-oxazinan-3-yl)phenyl)pyrimidin-2-yl)amino)cyclohexane-1-carboxylic acid (74.9 mg, 181 μmol, 0.500 eq) in DMF (6 mL) were added HATU (48.1 mg, 126 μmol, 0.350 eq), DIEA (234 mg, 1.81 mmol, 315 μL, 5.00 eq) and 3-((4-(4-amino-3′,3′-difluoro-[1,4′-bipiperidin]-1′-yl)-3-fluorophenyl)amino)piperidine-2,6-dione (200 mg, 361 μmol, 1.00 eq, TFA) at 25 °C. The mixture was stirred at 25 °C for 1 h. LCMS showed 3-((4-(4-amino-3′,3′-difluoro-[1,4′-bipiperidin]-1′-yl)-3-fluorophenyl)amino)piperidine-2,6-dione was consumed completely and the desired mass was detected. The reaction mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 35%-65% B over 8.0 min) to give the title compound (Compound 495) (28.1 mg, 32.7 μmol, 9.05% yield, 97.3% purity) as a white solid. LCMS (ES⁺): m/z 836.4 [M+H]⁺

### Example 494. Synthesis of trans-N-(1′-(4-((2,6-dioxopiperidin-3-yl)amino)-2-fluorophenyl)-[1,4′-bipiperidin]-4-yl)-4-((5-fluoro-4-(3-(2-oxo-1,3-oxazinan-3-yl)phenyl)pyrimidin-2-yl)amino)cyclohexane-1-carboxamide (Compound 522)

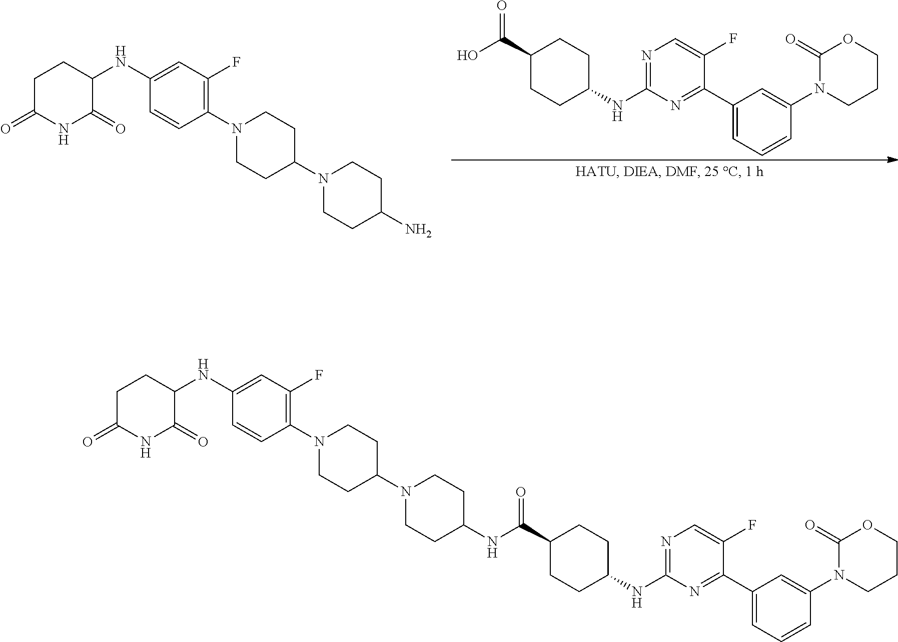 [SMILES: NC1CCN(C2CCN(c3ccc(NC4CCC(=O)NC4=O)cc3F)CC2)CC1 ; O=C1OCCCN1c1cccc(-c2nc(N[C@H]3CC[C@H](C(=O)O)CC3)ncc2F)c1 ; O=C1CCC(Nc2ccc(N3CCC(N4CCC(NC(=O)[C@H]5CC[C@H](Nc6ncc(F)c(-c7cccc(N8CCCOC8=O)c7)n6)CC5)CC4)CC3)c(F)c2)C(=O)N1]

Synthesis of trans-N-(1′-(4-((2,6-dioxopiperidin-3-yl)amino)-2-fluorophenyl)-[1,4′-bipiperidin]-4-yl)-4-((5-fluoro-4-(3-(2-oxo-1,3-oxazinan-3-yl)phenyl)pyrimidin-2-yl)amino)cyclohexane-1-carboxamide (Compound 522). To a stirred solution of trans-4-((5-fluoro-4-(3-(2-oxo-1,3-oxazinan-3-yl)phenyl)pyrimidin-2-yl)amino)cyclohexane-1-carboxylic acid (82.1 mg, 198 μmol, 0.500 eq) and DIEA (154 mg, 1.19 mmol, 205 μL, 3.00 eq) in DMF (5 mL) were added HATU (181 mg, 475 μmol, 1.20 eq) and 3-((4-(4-amino-[1,4′-bipiperidin]-1′-yl)-3-fluorophenyl)amino)piperidine-2,6-dione (205 mg, 396 μmol, 1.00 eq, TFA) at 25 °C. The resulting mixture was stirred at 25 °C for 1 h. LCMS showed 3-((4-(4-amino-[1,4′-bipiperidin]-1′-yl)-3-fluorophenyl)amino)piperidine-2,6-dione was consumed completely and the desired mass was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 18%-48% B over 8.0 min) to give the title compound (Compound 522) (43.9 mg, 51.0 μmol, 12.9% yield, 93.0% purity) as a white solid. LCMS (ES⁺): m/z 800.2 [M+H]⁺.

### Example 495. Synthesis of trans-(1r,4R)-4-(4-(4-((2,6-dioxopiperidin-3-yl)amino)-2-fluorophenyl)piperazin-1-yl)-N-((1r,4R)-4-((5-fluoro-4-(3-(2-oxooxazolidin-3-yl)phenyl)pyrimidin-2-yl)amino)cyclohexyl)cyclohexane-1-carboxamide (Compound 411)

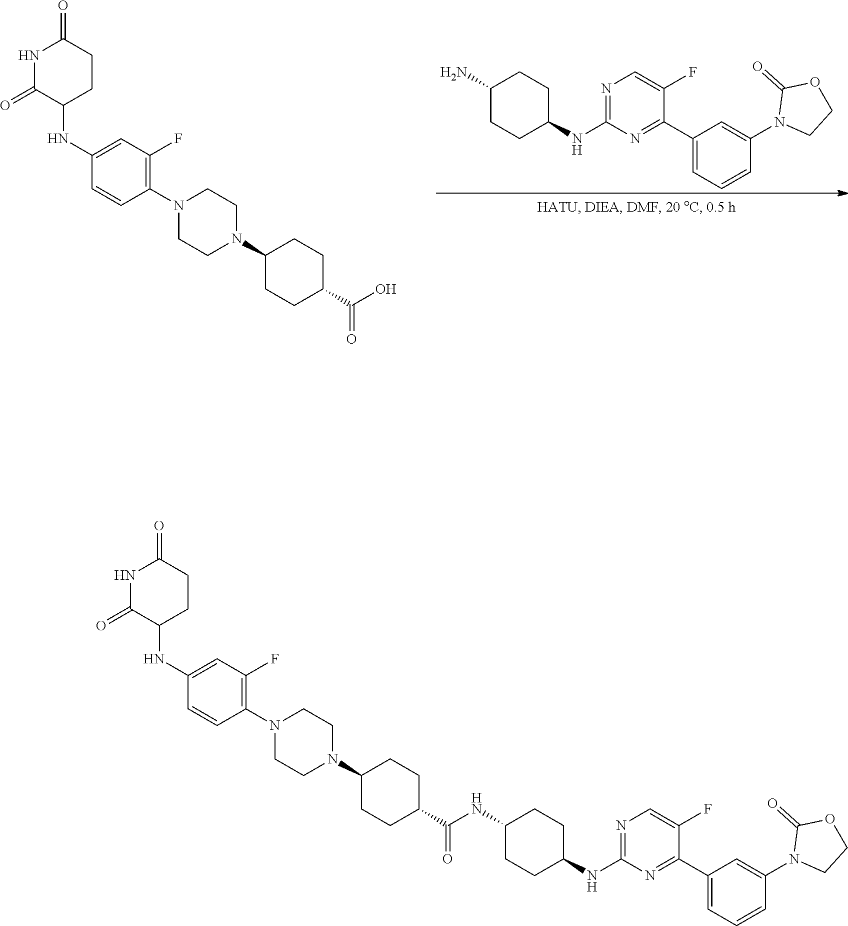 [SMILES: O=C1CCC(Nc2ccc(N3CCN([C@H]4CC[C@H](C(=O)O)CC4)CC3)c(F)c2)C(=O)N1 ; N[C@H]1CC[C@H](Nc2ncc(F)c(-c3cccc(N4CCOC4=O)c3)n2)CC1 ; O=C1CCC(Nc2ccc(N3CCN([C@H]4CC[C@H](C(=O)N[C@H]5CC[C@H](Nc6ncc(F)c(-c7cccc(N8CCOC8=O)c7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperazin-1-yl)-N-((1r,4R)-4-((5-fluoro-4-(3-(2-oxooxazolidin-3-yl)phenyl)pyrimidin-2-yl)amino)cyclohexyl)cyclohexane-1-carboxamide (Compound 411). To a solution of trans-4-(4-(4-((2,6-dioxopiperidin-3-yl)amino)-2-fluorophenyl)piperazin-1-yl)cyclohexane-1-carboxylic acid (500 mg, 1.08 mmol, 1.00 eq) in DMF (4 mL) were added HATU (123 mg, 323 μmol, 0.300 eq), DIEA (695 mg, 5.38 mmol, 936 μL, 5.00 eq) and trans-3-(3-(2-((4-aminocyclohexyl)amino)-5-fluoropyrimidin-4-yl)phenyl)oxazolidin-2-one (209 mg, 430 μmol, 0.400 eq, TFA) at 20 °C, the mixture was stirred at 20 °C for 0.5 h. LCMS showed trans-4-(4-(4-((2,6-dioxopiperidin-3-yl)amino)-2-fluorophenyl)piperazin-1-yl)cyclohexane-1-carboxylic acid was consumed completely and the desired target MS was detected. The mixture was concentrated under reduced pressure to give a residue. The residue was purified by prep-HPLC (column: Phenomenex luna C18 100*40 mm*3 um; mobile phase: [H₂O (0.200% FA)-ACN]; gradient: 2%-37% B over 8.0 min) to give the title compound (Compound 411) (75.6 mg, 91.5 μmol, 8.51% yield, 95.1% purity) as a yellow solid. LCMS (ES⁺): m/z 786.4 [M+H]⁺

### Example 496. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(3-(2-oxooxazolidin-3-yl)phenyl)pyrimidin-2-yl)amino)cyclohexyl)cyclohexane-1-carboxamide (Compound 446)

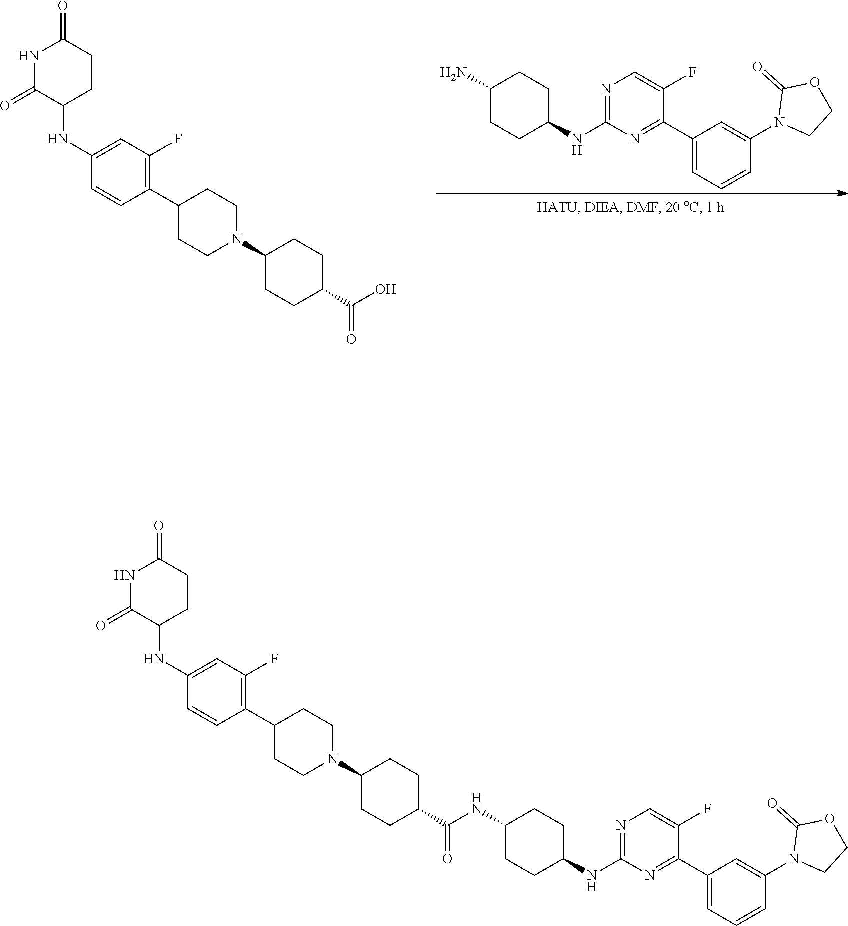 [SMILES: O=C1CCC(Nc2ccc(C3CCN([C@H]4CC[C@H](C(=O)O)CC4)CC3)c(F)c2)C(=O)N1 ; N[C@H]1CC[C@H](Nc2ncc(F)c(-c3cccc(N4CCOC4=O)c3)n2)CC1 ; O=C1CCC(Nc2ccc(C3CCN([C@H]4CC[C@H](C(=O)N[C@H]5CC[C@H](Nc6ncc(F)c(-c7cccc(N8CCOC8=O)c7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(3-(2-oxooxazolidin-3-yl)phenyl)pyrimidin-2-yl)amino)cyclohexyl)cyclohexane-1-carboxamide (Compound 446). To a solution of trans-4-(4-(4-((2,6-dioxopiperidin-3-yl)amino)-2-fluorophenyl)piperidin-1-yl)cyclohexane-1-carboxylic acid (398 mg, 922 μmol, 1.00 eq) in DMF (5 mL) were added HATU (140 mg, 369 μmol, 0.400 eq) and DIEA (609 mg, 4.62 mmol, 819 μL, 5.00 eq) at 20 °C, then 3-(3-(2-((4-aminocyclohexyl)amino)-5-fluoropyrimidin-4-yl)phenyl)oxazolidin-2-one (179 mg, 369 μmol, 0.400 eq, TFA) was added to the mixture, the mixture was stirred at 20 °C for 1 h. LCMS showed trans-4-(4-(4-((2,6-dioxopiperidin-3-yl)amino)-2-fluorophenyl)piperidin-1-yl)cyclohexane-1-carboxylic acid was consumed and the desired target MS was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 10%-40% B over 8.0 min) to give the title compound (Compound 446) (75.6 mg, 90.4 μmol, 9.80% yield, 93.9% purity) as a pale yellow solid. LCMS (ES⁺): m/z 785.4 [M+H]⁺

### Example 497. Synthesis of trans-′-(4-((2,6-dioxopiperidin-3-yl)amino)-2-fluorophenyl)-N-(4-((5-fluoro-4-(3-(2-oxooxazolidin-3-yl)phenyl)pyrimidin-2-yl)amino)cyclohexyl)-[1,4′-bipiperidine]-4-carboxamide (Compound 596)

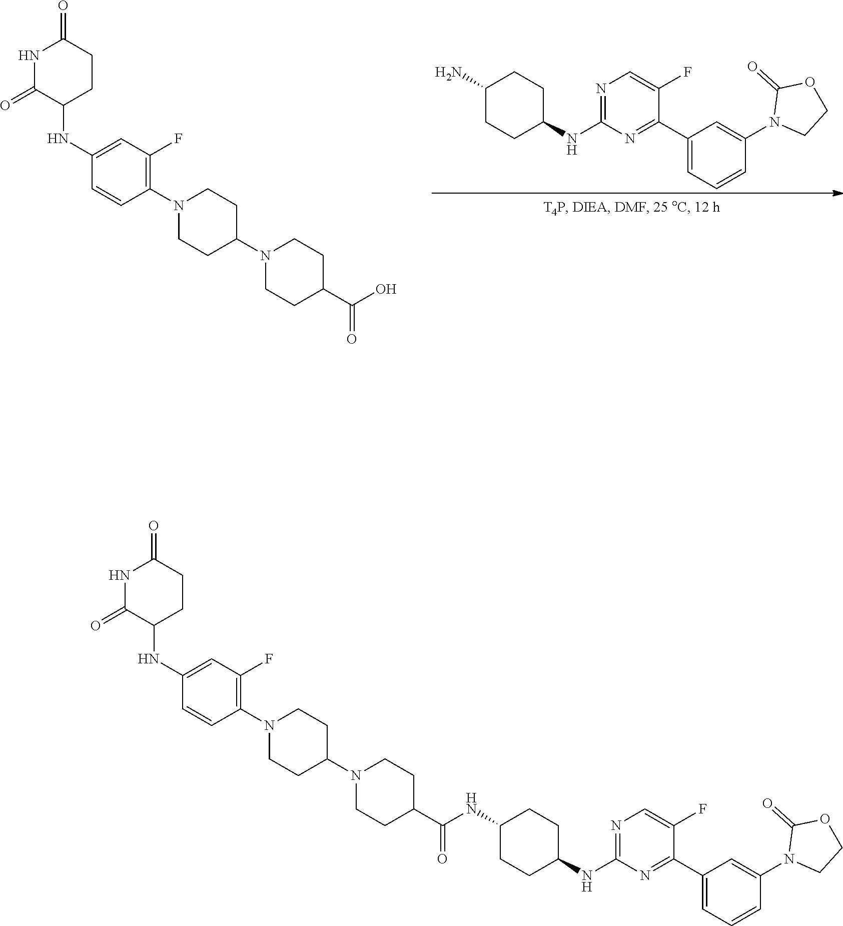 [SMILES: O=C1CCC(Nc2ccc(N3CCC(N4CCC(C(=O)O)CC4)CC3)c(F)c2)C(=O)N1 ; N[C@H]1CC[C@H](Nc2ncc(F)c(-c3cccc(N4CCOC4=O)c3)n2)CC1 ; O=C1CCC(Nc2ccc(N3CCC(N4CCC(C(=O)N[C@H]5CC[C@H](Nc6ncc(F)c(-c7cccc(N8CCOC8=O)c7)n6)CC5)CC4)CC3)c(F)c2)C(=O)N1]

Synthesis of trans-1′-(4-((2,6-dioxopiperidin-3-yl)amino)-2-fluorophenyl)-N-(4-((5-fluoro-4-(3-(2-oxooxazolidin-3-yl)phenyl)pyrimidin-2-yl)amino)cyclohexyl)-[1,4′-bipiperidine]-4-carboxamide (Compound 596). To a solution of 1-[1-[4-[(2,6-dioxo-3-piperidyl)amino]-2-fluoro-phenyl]-4-piperidyl]piperidine-4-carboxylic acid (1.00 g, 1.83 mmol, 1.00 eq, TFA) in DMF (2 mL) was added DIEA (1.17 g, 9.09 mmol, 1.58 mL, 4.97 eq) until pH~7. To a solution of trans-3-(3-(2-((4-aminocyclohexyl)amino)-5-fluoropyrimidin-4-yl)phenyl)oxazolidin-2-one (395 mg, 815 μmol, 0.445 eq, TFA) in DMF (2 mL) was added DIEA (1.17 g, 9.09 mmol, 1.58 mL, 4.97 eq) until pH~7. Then T₄P (1.32 g, 3.66 mmol, 50%, 2.00 eq) was added to the mixture of the two solutions. The mixture was stirred at 25 °C for 12 h. LC-MS showed the desired mass was detected. The reaction mixture was filtered. The residue was purified by prep-HPLC (column: Waters Xbridge BEH C18 100*30 mm*10 um; mobile phase: [H₂O (10 mM NH₄HCO₃)-ACN]; gradient: 30%-60% B over 8.0 min) to give the title compound (Compound 596) (71.6 mg, 88.7 μmol, 4.85% yield, 97.4% purity) as a pale yellow solid. LCMS (ES⁺): m/z 786.4 [M+H]⁺.

### Example 498. Synthesis of trans-(1r,4R)-4-(4-(4-((2,6-dioxopiperidin-3-yl)amino)-2-fluorophenyl)piperazin-1-yl)-N-((1r,4R)-4-((5-fluoro-4-(3-(2-oxopiperidin-1-yl)phenyl)pyrimidin-2-yl)amino)cyclohexyl)cyclohexane-1-carboxamide (Compound 412)

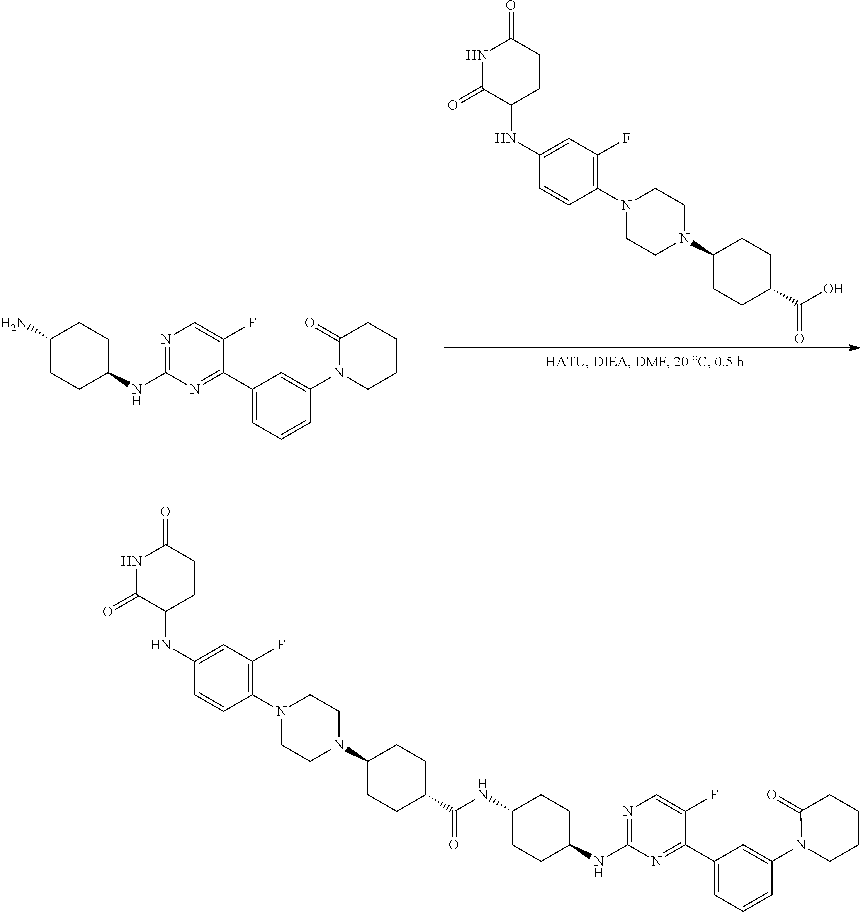 [SMILES: N[C@H]1CC[C@H](Nc2ncc(F)c(-c3cccc(N4CCCCC4=O)c3)n2)CC1 ; O=C1CCC(Nc2ccc(N3CCN([C@H]4CC[C@H](C(=O)O)CC4)CC3)c(F)c2)C(=O)N1 ; O=C1CCC(Nc2ccc(N3CCN([C@H]4CC[C@H](C(=O)N[C@H]5CC[C@H](Nc6ncc(F)c(-c7cccc(N8CCCCC8=O)c7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperazin-1-yl)-N-((1r,4R)-4-((5-fluoro-4-(3-(2-oxopiperidin-1-yl)phenyl)pyrimidin-2-yl)amino)cyclohexyl)cyclohexane-1-carboxamide (Compound 412). To a solution of trans-4-(4-(4-((2,6-dioxopiperidin-3-yl)amino)-2-fluorophenyl)piperazin-1-yl)cyclohexane-1-carboxylic acid (500 mg, 1.08 mmol, 1.00 eq) in DMF (10 mL) were added HATU (123 mg, 323 μmol, 0.300 eq), DIEA (695 mg, 5.38 mmol, 936 μL, 5.00 eq) and trans-1-(3-(2-((4-aminocyclohexyl)amino)-5-fluoropyrimidin-4-yl)phenyl)piperidin-2-one (214 mg, 430 μmol, 0.400 eq, TFA) at 20 °C, the mixture was stirred at 20 °C for 0.5 h. LCMS showed trans-4-(4-(4-((2,6-dioxopiperidin-3-yl)amino)-2-fluorophenyl)piperazin-1-yl)cyclohexane-1-carboxylic acid was consumed completely and the desired target MS was detected. The mixture was concentrated under reduced pressure to give a residue. The residue was purified by prep-HPLC (column: Phenomenex Luna C18 75*30 mm*3 um; mobile phase: [H₂O (0.2% FA)-ACN]; gradient: 10%-50% B over 8.0 min) to give the title compound (Compound 412) (59.9 mg, 68.4 μmol, 6.36% yield, 91.1% purity) as a yellow solid. LCMS (ES⁺): m/z 798.4 [M+H]⁺

### Example 499. Synthesis of trans-(1R,4R)-4-(4-(4-(((R)-2,6-dioxopiperidin-3-yl)amino)-2-fluorophenyl)piperazin-1-yl)-N-((1r,4R)-4-((5-fluoro-4-(3-(2-oxopiperidin-1-yl)phenyl)pyrimidin-2-yl)amino)cyclohexyl)cyclohexane-1-carboxamide (Compound 445) and trans-(1S,4R)-4-(4-(4-(((S)-2,6-dioxopiperidin-3-yl)amino)-2-fluorophenyl)piperazin-1-yl)-N-((1r,4R)-4-((5-fluoro-4-(3-(2-oxopiperidin-1-yl)phenyl)pyrimidin-2-yl)amino)cyclohexyl)cyclohexane-1-carboxamide (Compound 458) (The Two Isomers were Assigned Arbitrarily)

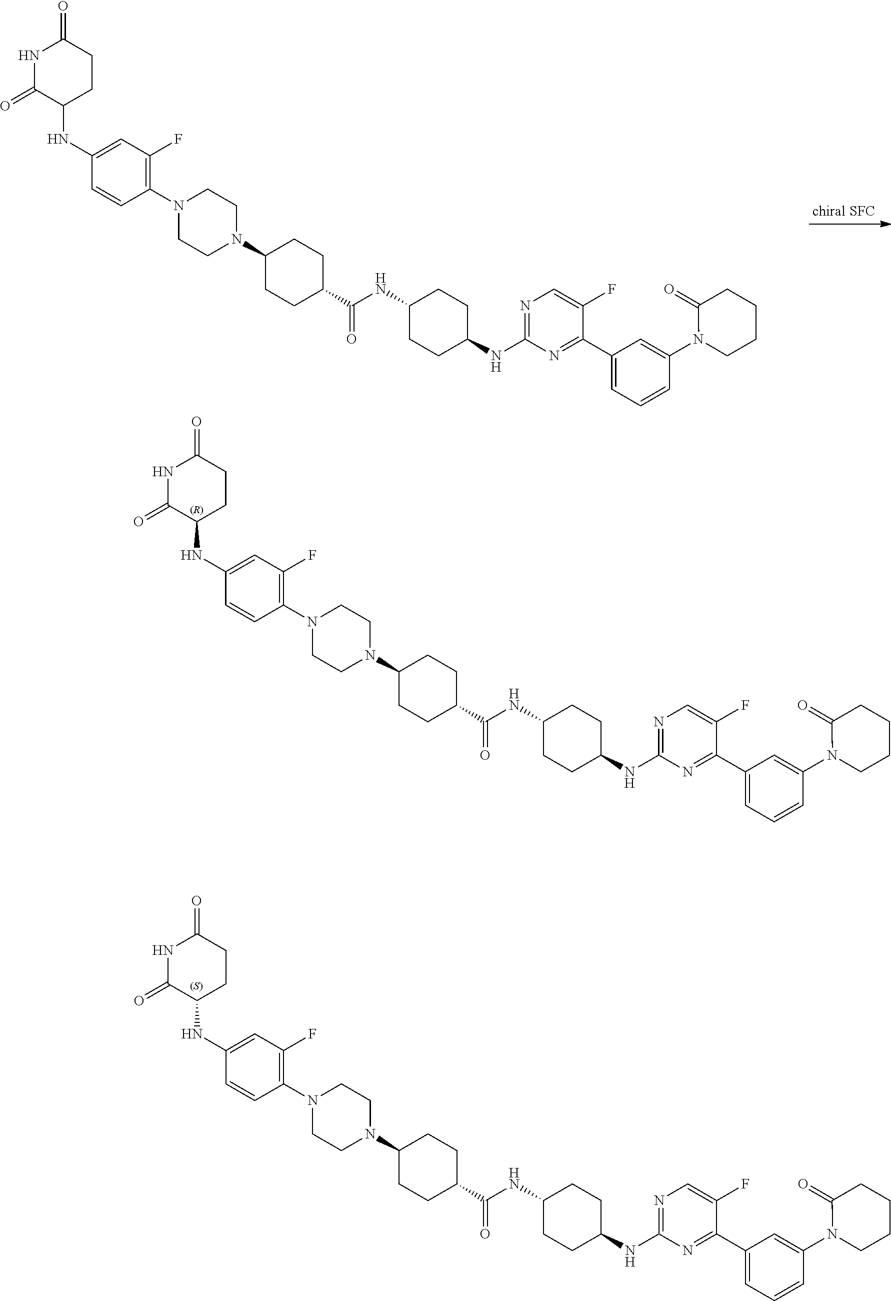

Synthesis of trans-(1R,4R)-4-(4-(4-(((R)-2,6-dioxopiperidin-3-yl)amino)-2-fluorophenyl)piperazin-1-yl)-N-((1r,4R)-4-((5-fluoro-4-(3-(2-oxopiperidin-1-yl)phenyl)pyrimidin-2-yl)amino)cyclohexyl)cyclohexane-1-carboxamide (Compound 445) and trans-(1S,4R)-4-(4-(4-(((S)-2,6-dioxopiperidin-3-yl)amino)-2-fluorophenyl)piperazin-1-yl)-N-((1r,4R)-4-((5-fluoro-4-(3-(2-oxopiperidin-1-yl)phenyl)pyrimidin-2-yl)amino)cyclohexyl)cyclohexane-1-carboxamide (Compound 458) (the two isomers were assigned arbitrarily). Trans-(1r,4R)-4-(4-(4-((2,6-dioxopiperidin-3-yl)amino)-2-fluorophenyl)piperazin-1-yl)-N-((1r,4R)-4-((5-fluoro-4-(3-(2-oxopiperidin-1-yl)phenyl)pyrimidin-2-yl)amino)cyclohexyl)cyclohexane-1-carboxamide (Compound 412) (50.0 mg, 62.7 μmol, 1.00 eq) was separated by chiral SFC (column: ChiralPak IH, 250*30 mm, 10 um; mobile phase: [CO₂—IPA:CAN=1:1 (0.1% NH₃H₂O)]; B %: 55%, isocratic elution mode) to give the title compound (Compound 445) (25.9 mg, 32.5 μmol, 51.8% yield, 100% purity, 100% ee) (retention time: 1.505, peak 1) as an off-white solid. LCMS (ES⁺): m/z 798.4 [M+H]⁺ and the title compound (Compound 458) (16.1 mg, 19.1 μmol, 30.4% yield, 94.4% purity, 91.1% ee) (retention time: 2.460, peak 2) as a pale yellow solid. LCMS (ES⁺): m/z 798.4 [M+H]⁺

### Example 500. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(3-(2-oxopiperidin-1-yl)phenyl)pyrimidin-2-yl)amino)cyclohexyl)cyclohexane-1-carboxamide (Compound 447)

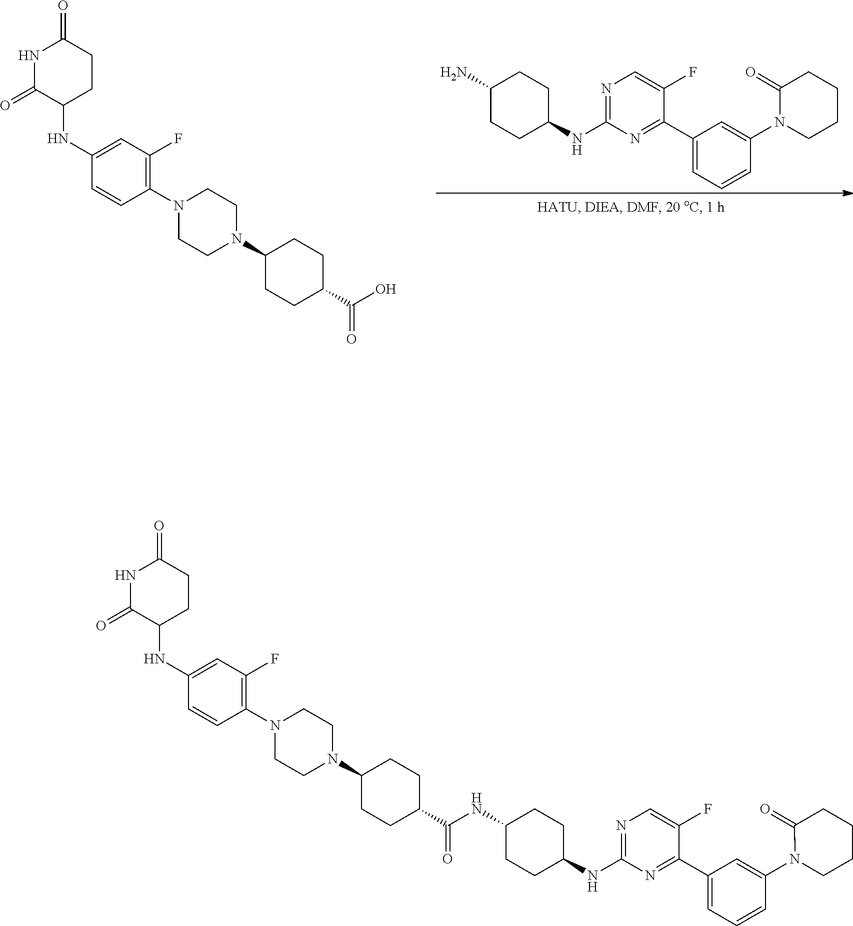 [SMILES: O=C1CCC(Nc2ccc(N3CCN([C@H]4CC[C@H](C(=O)O)CC4)CC3)c(F)c2)C(=O)N1 ; N[C@H]1CC[C@H](Nc2ncc(F)c(-c3cccc(N4CCCCC4=O)c3)n2)CC1 ; O=C1CCC(Nc2ccc(N3CCN([C@H]4CC[C@H](C(=O)N[C@H]5CC[C@H](Nc6ncc(F)c(-c7cccc(N8CCCCC8=O)c7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperidin-1-yl)-N-((1 r,4R)-4-((5-fluoro-4-(3-(2-oxopiperidin-1-yl)phenyl)pyrimidin-2-yl)amino)cyclohexyl)cyclohexane-1-carboxamide (Compound 447). To a solution of trans-4-(4-(4-((2,6-dioxopiperidin-3-yl)amino)-2-fluorophenyl)piperidin-1-yl)cyclohexane-1-carboxylic acid (420 mg, 973 μmol, 1.00 eq) in DMF (5 mL) were added HATU (111 mg, 292 μmol, 0.300 eq) and DIEA (641 mg, 4.87 mmol, 865 μL, 5.00 eq) at 20 °C, then trans-1-(3-(2-((4-aminocyclohexyl)amino)-5-fluoropyrimidin-4-yl)phenyl)piperidin-2-one (194 mg, 389 μmol, 0.400 eq, TFA) was added to the mixture, the mixture was stirred at 20 °C for 1 h. LCMS showed trans-4-(4-(4-((2,6-dioxopiperidin-3-yl)amino)-2-fluorophenyl)piperidin-1-yl)cyclohexane-1-carboxylic acid was consumed and the desired target MS was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 25%-55% B over 8.0 min) to give the title compound (Compound 447) (91.9 mg, 112 μmol, 11.5% yield, 96.8% purity) as an off-white solid. LCMS (ES⁺): m/z 797.4 [M+H]⁺

### Example 501. Synthesis of trans-(1R,4R)-4-(4-(4-(((R)-2,6-dioxopiperidin-3-yl)amino)-2-fluorophenyl)piperidin-1-yl)-N-((1r,4R)-4-((5-fluoro-4-(3-(2-oxopiperidin-1-yl)phenyl)pyrimidin-2-yl)amino)cyclohexyl)cyclohexane-1-carboxamide (Compound 593) and trans-(1S,4R)-4-(4-(4-(((S)-2,6-dioxopiperidin-3-yl)amino)-2-fluorophenyl)piperidin-1-yl)-N-((1r,4R)-4-((5-fluoro-4-(3-(2-oxopiperidin-1-yl)phenyl)pyrimidin-2-yl)amino)cyclohexyl)cyclohexane-1-carboxamide (Compound 592) (The Two Isomers were Assigned Arbitrarily)

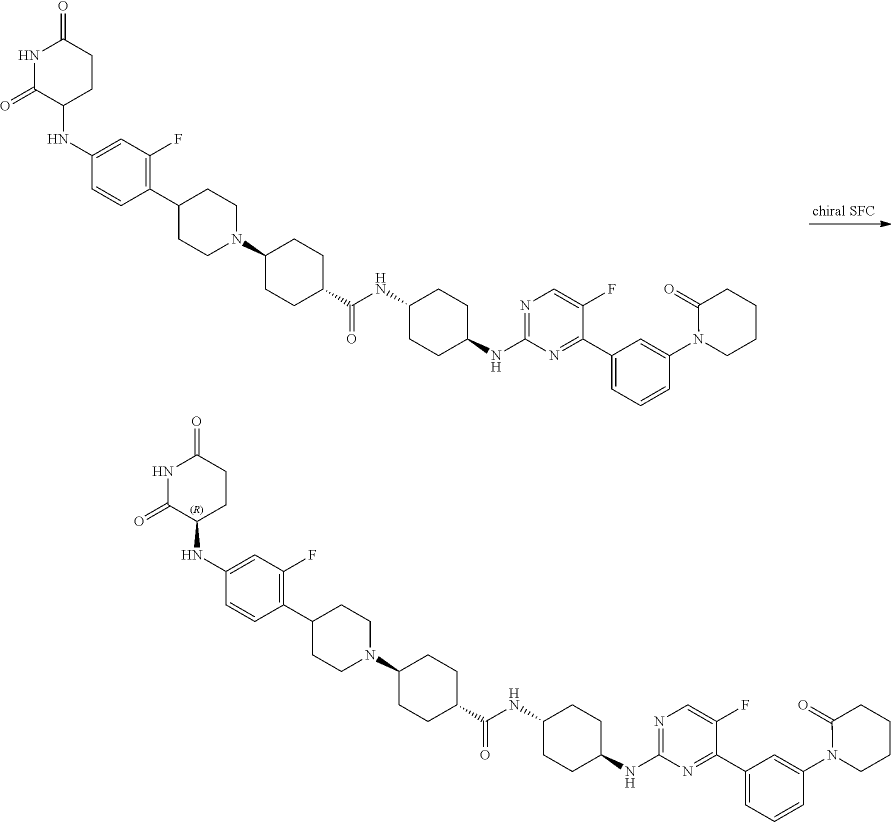

Synthesis of trans-(1R,4R)-4-(4-(4-(((R)-2,6-dioxopiperidin-3-yl)amino)-2-fluorophenyl)piperidin-1-yl)-N-((1r,4R)-4-((5-fluoro-4-(3-(2-oxopiperidin-1-yl)phenyl)pyrimidin-2-yl)amino)cyclohexyl)cyclohexane-1-carboxamide (Compound 593) and trans-(1S,4R)-4-(4-(4-(((S)-2,6-dioxopiperidin-3-yl)amino)-2-fluorophenyl)piperidin-1-yl)-N-((1r,4R)-4-((5-fluoro-4-(3-(2-oxopiperidin-1-yl)phenyl)pyrimidin-2-yl)amino)cyclohexyl)cyclohexane-1-carboxamide (Compound 592) (the two isomers were assigned arbitrarily). Trans-(1r,4R)-4-(4-(4-((2,6-dioxopiperidin-3-yl)amino)-2-fluorophenyl)piperidin-1-yl)-N-((1r,4R)-4-((5-fluoro-4-(3-(2-oxopiperidin-1-yl)phenyl)pyrimidin-2-yl)amino)cyclohexyl)cyclohexane-1-carboxamide (Compound 447) (86.0 mg, 108 μmol, 1.00 eq) was separated by chiral SFC (column: AZT-SB1012 C18 10 μm 120 A 250*50 mm; mobile phase: [Heptane-IPA:CAN=4:1]; B %: 50%, isocratic elution mode) to give the title compound (Compound 593) (12.1 mg, 14.3 μmol, 13.2% yield, 94.0% purity, 95.0% ee) (retention time: 3.912, peak 1) as a white solid. LCMS (ES⁺): m/z 797.4 [M+H]⁺ and the title compound (Compound 592) (12.6 mg, 15.5 μmol, 14.3% yield, 97.8% purity, 91.9% ee) (retention time: 4.718, peak 2) as a white solid. LCMS (ES⁺): m/z 797.4 [M+H]⁺

### Example 502 Synthesis of 1′-(4-((2,6-dioxopiperidin-3-yl)amino)-2-fluorophenyl)-N-(trans-4-((5-fluoro-4-(3-(2-oxopiperidin-1-yl)phenyl)pyrimidin-2-yl)amino)cyclohexyl)-[1,4′-bipiperidine]-4-carboxamide (Compound 587)

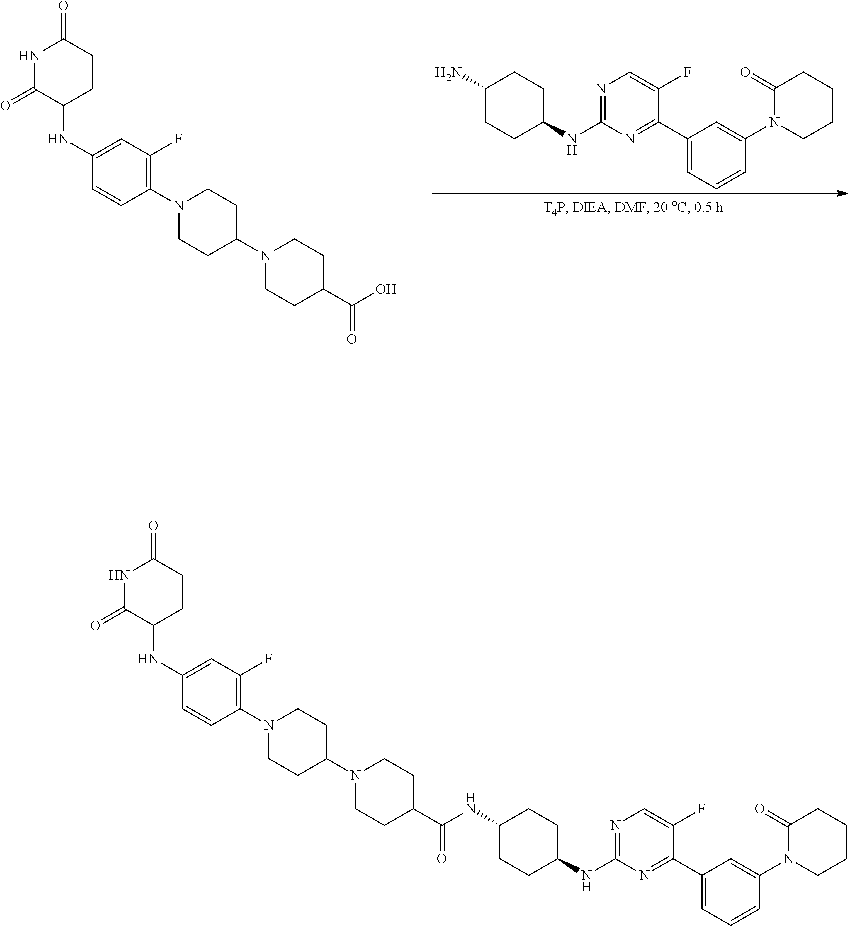 [SMILES: O=C1CCC(Nc2ccc(N3CCC(N4CCC(C(=O)O)CC4)CC3)c(F)c2)C(=O)N1 ; N[C@H]1CC[C@H](Nc2ncc(F)c(-c3cccc(N4CCCCC4=O)c3)n2)CC1 ; O=C1CCC(Nc2ccc(N3CCC(N4CCC(C(=O)N[C@H]5CC[C@H](Nc6ncc(F)c(-c7cccc(N8CCCCC8=O)c7)n6)CC5)CC4)CC3)c(F)c2)C(=O)N1]

Synthesis of 1′-(4-((2,6-dioxopiperidin-3-yl)amino)-2-fluorophenyl)-N-(trans-4-((5-fluoro-4-(3-(2-oxopiperidin-1-yl)phenyl)pyrimidin-2-yl)amino)cyclohexyl)-[1,4′-bipiperidine]-4-carboxamide (Compound 587). The reaction was set up in two batches. To a solution of trans-1-[3-[2-[(4-aminocyclohexyl)amino]-5-fluoro-pyrimidin-4-yl]phenyl]piperidin-2-one (400 mg, 952 μmol, 1.00 eq, HCl) and 1-[1-[4-[(2,6-dioxo-3-piperidyl)amino]-2-fluoro-phenyl]-4-piperidyl]piperidine-4-carboxylic acid (520 mg, 952 μmol, 1.00 eq, TFA) in DMF (2 mL) was added DIEA (246 mg, 1.91 mmol, 331 μL, 2.00 eq) and T₄P (1.37 g, 1.91 mmol, 50% purity, 2.00 eq) at 20 °C. The mixture was stirred at 20 °C for 0.5 h. LC-MS showed the desired mass was detected. Two batches in parallel were combined and the reaction mixture was diluted with H₂O 3 mL and extracted with EtOAc 3 mL*3. The combined organic layers were dried over Na₂SO₄, filtered and concentrated under reduced pressure. The residue was purified by prep-HPLC (column: Waters Xbridge BEH C18 250*50 mm*10 um; mobile phase: [H₂O (10 mM NH₄HCO₃)-ACN]; gradient: 25%-55% B over 10.0 min) to give the title compound (Compound 587) (214 mg, 269 μmol, 14.1% yield, 100% purity) as a gray solid. LCMS (ES⁺): m/z 798.4 [M+H]⁺.

### Example 503. Synthesis of trans-2-((1r,4R)-4-((4-(4-((2,6-dioxopiperidin-3-yl)amino)-2-fluorophenyl)piperazin-1-yl)methyl)cyclohexyl)-N-((1r,4R)-4-((5-fluoro-4-(3-(2-oxopiperidin-1-yl)phenyl)pyrimidin-2-yl)amino)cyclohexyl)acetamide (Compound 560)

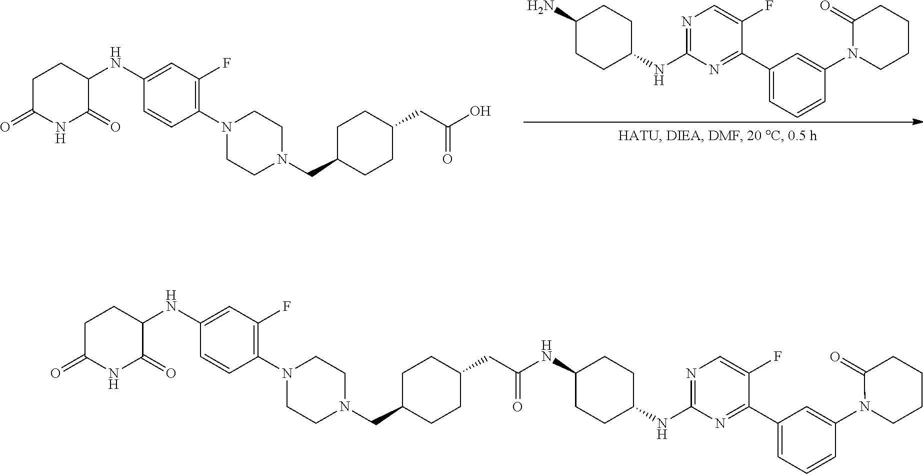 [SMILES: O=C(O)C[C@H]1CC[C@H](CN2CCN(c3ccc(NC4CCC(=O)NC4=O)cc3F)CC2)CC1 ; N[C@H]1CC[C@H](Nc2ncc(F)c(-c3cccc(N4CCCCC4=O)c3)n2)CC1 ; O=C1CCC(Nc2ccc(N3CCN(C[C@H]4CC[C@H](CC(=O)N[C@H]5CC[C@H](Nc6ncc(F)c(-c7cccc(N8CCCCC8=O)c7)n6)CC5)CC4)CC3)c(F)c2)C(=O)N1]

Synthesis of trans-2-((1r,4R)-4-((4-(4-((2,6-dioxopiperidin-3-yl)amino)-2-fluorophenyl)piperazin-1-yl)methyl)cyclohexyl)-N-((1r,4R)-4-((5-fluoro-4-(3-(2-oxopiperidin-1-yl)phenyl)pyrimidin-2-yl)amino)cyclohexyl)acetamide (Compound 560). To a solution of trans-2-(4-((4-(4-((2,6-dioxopiperidin-3-yl)amino)-2-fluorophenyl)piperazin-1-yl)methyl)cyclohexyl)acetic acid (445 mg, 966 μmol, 1.00 eq) in DMF (8 mL) were added HATU (129 mg, 338 μmol, 0.350 eq) and DIEA (625 mg, 4.83 mmol, 842 μL, 5.00 eq) at 20 °C. Then to the mixture was added trans-1-(3-(2-((4-aminocyclohexyl)amino)-5-fluoropyrimidin-4-yl)phenyl)piperidin-2-one (288 mg, 580 μmol, 0.600 eq, TFA) and the resulting mixture was stirred at 20 °C for 0.5 h. LCMS showed trans-2-(4-((4-(4-((2,6-dioxopiperidin-3-yl)amino)-2-fluorophenyl)piperazin-1-yl)methyl)cyclohexyl)acetic acid was consumed and the desired target MS was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 35%-75% B over 8.0 min) to give the title compound (Compound 560) (137 mg, 164 μmol, 17.0% yield, 99.0% purity) as a pale yellow solid. LCMS (ES⁺): m/z 826.4 [M+H]⁺

### Example 504. Synthesis of 1′-(4-((2,6-dioxopiperidin-3-yl)amino)-2-fluorophenyl)-3′-fluoro-N-((1r,4r)-4-((5-fluoro-4-(3-(2-oxopiperidin-1-yl)phenyl)pyrimidin-2-yl)amino)cyclohexyl)-[1,4′-bipiperidine]-4-carboxamide (Compound 524)

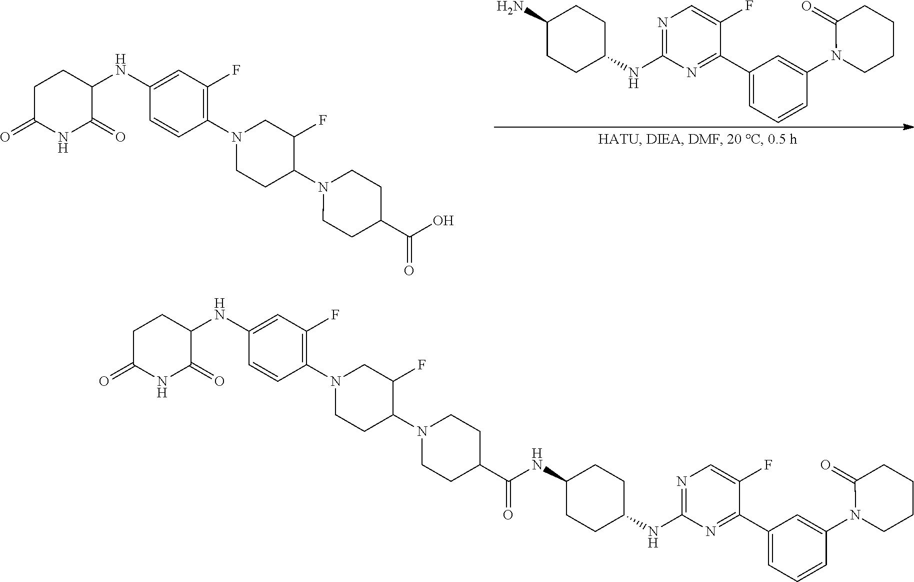 [SMILES: O=C1CCC(Nc2ccc(N3CCC(N4CCC(C(=O)O)CC4)C(F)C3)c(F)c2)C(=O)N1 ; N[C@H]1CC[C@H](Nc2ncc(F)c(-c3cccc(N4CCCCC4=O)c3)n2)CC1 ; O=C1CCC(Nc2ccc(N3CCC(N4CCC(C(=O)N[C@H]5CC[C@H](Nc6ncc(F)c(-c7cccc(N8CCCCC8=O)c7)n6)CC5)CC4)C(F)C3)c(F)c2)C(=O)N1]

Synthesis of 1′-(4-((2,6-dioxopiperidin-3-yl)amino)-2-fluorophenyl)-3′-fluoro-N-((1r,4r)-4-((5-fluoro-4-(3-(2-oxopiperidin-1-yl)phenyl)pyrimidin-2-yl)amino)cyclohexyl)-[1,4′-bipiperidine]-4-carboxamide (Compound 524). To a solution of 1′-(4-((2,6-dioxopiperidin-3-yl)amino)-2-fluorophenyl)-3′-fluoro-[1,4′-bipiperidine]-4-carboxylic acid (266 mg, 590 μmol, 1.00 eq) and trans-1-(3-(2-((4-aminocyclohexyl)amino)-5-fluoropyrimidin-4-yl)phenyl)piperidin-2-one (147 mg, 295 μmol, 0.500 eq, TFA) in DMF (5 mL) were added HATU (78.6 mg, 207 μmol, 0.350 eq) and DIEA (498 mg, 2.95 mmol, 514 μL, 5.00 eq) at 20 °C, then the mixture was stirred at 20 °C for 0.5 h. LCMS showed 1′-(4-((2,6-dioxopiperidin-3-yl)amino)-2-fluorophenyl)-3′-fluoro-[1,4′-bipiperidine]-4-carboxylic acid was consumed completely and the desired target MS was detected. The mixture was concentrated under reduced pressure to give a residue. The residue was purified by prep-HPLC ACN]; gradient: 15%-55% B over 8.0 min) to give the title compound (Compound 524) (71.8 mg, 86.1 μmol, 14.6% yield, 99.0% purity, 0.2 FA) as a yellow solid. LCMS (ES⁺): m/z 816.2 [M+H]⁺

### Example 505. Synthesis of trans-1′-(4-((2,6-dioxopiperidin-3-yl)amino)-2-fluorophenyl)-3′,3′-difluoro-N-(4-((5-fluoro-4-(3-(2-oxopiperidin-1-yl)phenyl)pyrimidin-2-yl)amino)cyclohexyl)-[1,4′-bipiperidine]-4-carboxamide (Compound 499)

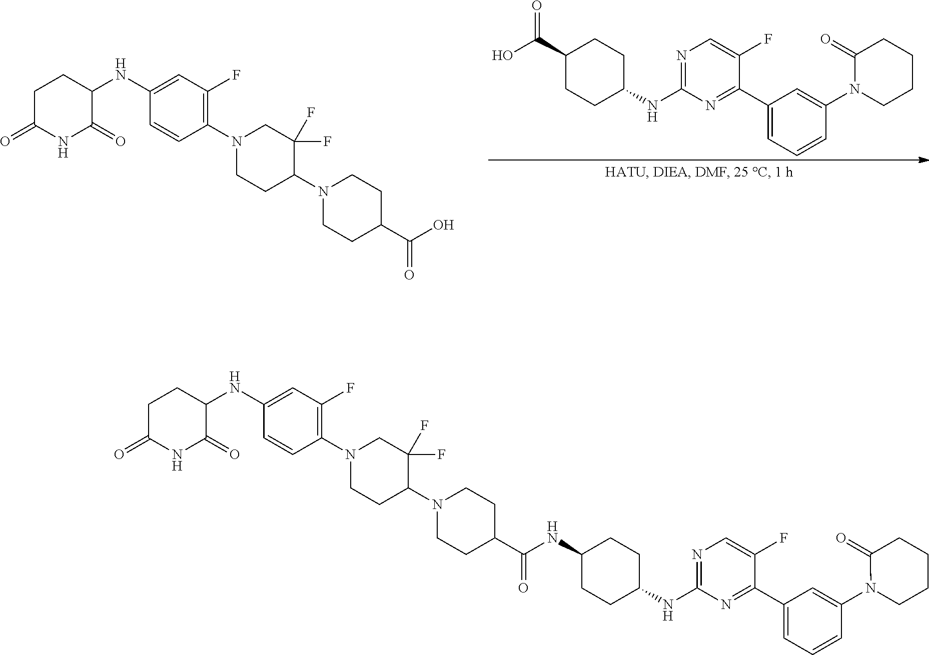

Synthesis of trans-1′-(4-((2,6-dioxopiperidin-3-yl)amino)-2-fluorophenyl)-3′,3′-difluoro-N-(4-((5-fluoro-4-(3-(2-oxopiperidin-1-yl)phenyl)pyrimidin-2-yl)amino)cyclohexyl)-[1,4′-bipiperidine]-4-carboxamide (Compound 499). To a solution of 1′-(4-((2,6-dioxopiperidin-3-yl)amino)-2-fluorophenyl)-3′,3′-difluoro-[1,4′-bipiperidine]-4-carboxylic acid (200 mg, 427 μmol, 1.00 eq) in DMF (7 mL) were added HATU (56.8 mg, 149 μmol, 0.350 eq), DIEA (275 mg, 2.14 mmol, 373 μL, 5.00 eq) and trans-1-(3-(2-((4-aminocyclohexyl)amino)-5-fluoropyrimidin-4-yl)phenyl)piperidin-2-one (85.0 mg, 171 μmol, 0.400 eq, TFA) at 25 °C. The mixture was stirred at 25 °C for 1 h. LCMS showed 1′-(4-((2,6-dioxopiperidin-3-yl)amino)-2-fluorophenyl)-3′,3′-difluoro-[1,4′-bipiperidine]-4-carboxylic acid was consumed completely and the desired mass was detected. The reaction mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 15%-50% B over 8.0 min) to give the title compound (Compound 499) (60.8 mg, 71.1 μmol, 16.7% yield, 97.5% purity) as a pale yellow solid. LCMS (ES⁺): m/z 834.5 [M+H]⁺.

### Example 506. Synthesis of trans-3-((3-fluoro-4-(4-((1-(4-((5-fluoro-4-(3-(2-oxopiperidin-1-yl)phenyl)pyrimidin-2-yl)amino)cyclohexane-1-carbonyl)piperidin-4-yl)oxy)piperidin-1-yl)phenyl)amino)piperidine-2,6-dione (Compound 413)

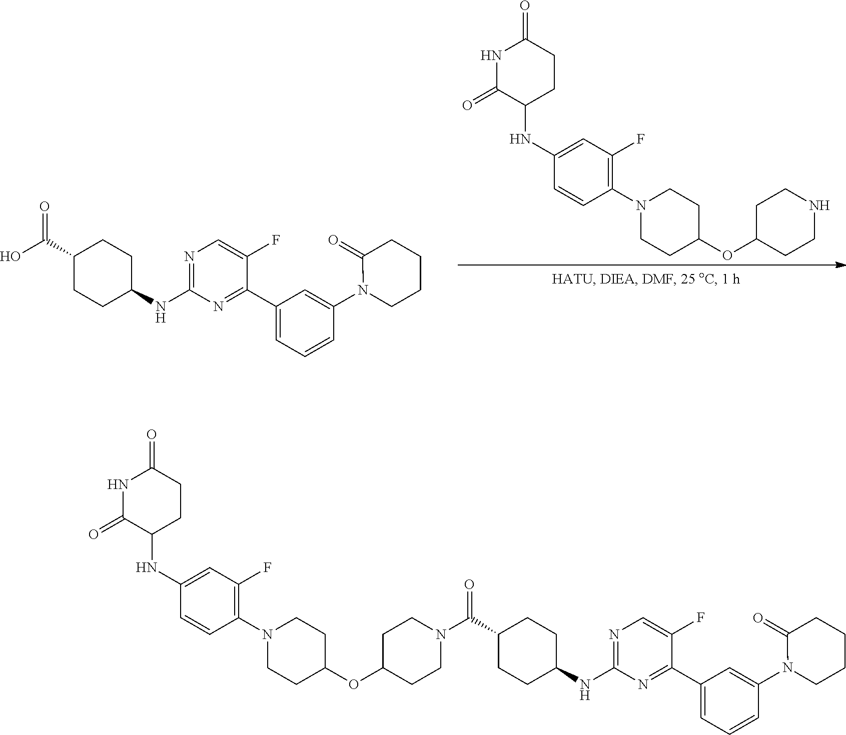 [SMILES: O=C1CCCCN1c1cccc(-c2nc(N[C@H]3CC[C@H](C(=O)O)CC3)ncc2F)c1 ; O=C1CCC(Nc2ccc(N3CCC(OC4CCNCC4)CC3)c(F)c2)C(=O)N1 ; O=C1CCC(Nc2ccc(N3CCC(OC4CCN(C(=O)[C@H]5CC[C@H](Nc6ncc(F)c(-c7cccc(N8CCCCC8=O)c7)n6)CC5)CC4)CC3)c(F)c2)C(=O)N1]

Synthesis of trans-3-((3-fluoro-4-(4-((1-(4-((5-fluoro-4-(3-(2-oxopiperidin-1-yl)phenyl)pyrimidin-2-yl)amino)cyclohexane-1-carbonyl)piperidin-4-yl)oxy)piperidin-1-yl)phenyl)amino)piperidine-2,6-dione (Compound 413). To a solution of trans-4-[[5-fluoro-4-[3-(2-oxo-1-piperidyl)phenyl]pyrimidin-2-yl]amino]cyclohexanecarboxylic acid (318 mg, 771 μmol, 0.500 eq) in DMF (8 mL) were added HATU (587 mg, 1.54 mmol, 1.00 eq) and DIEA (1.20 g, 9.26 mmol, 1.61 mL, 6.00 eq) at 25 °C, then 3-((3-fluoro-4-(4-(piperidin-4-yloxy)piperidin-1-yl)phenyl)amino)piperidine-2,6-dione (800 mg, 1.54 mmol, 1.00 eq, TFA) was added to the reaction mixture at 25 °C. The mixture was stirred at 25 °C for 1 h. LCMS showed 3-((3-fluoro-4-(4-(piperidin-4-yloxy)piperidin-1-yl)phenyl)amino)piperidine-2,6-dione was consumed completely and the desired mass was detected. The reaction mixture was filtered and the filtrate was concentrated under reduced pressure to give a residue. The residue was purified by prep-HPLC (column: Phenomenex luna C18 100*40 mm*5 um; mobile phase: [H₂O (0.2% FA)-ACN]; gradient: 15%-50% B over 8.0 min) to give the title compound (Compound 413) (112 mg, 135 μmol, 8.75% yield, 96.3% purity) as a gray solid. LCMS (ES⁺): m/z 799.5 [M+H]⁺

### Example 507. Synthesis of trans-3-((3-fluoro-4-(4-((1-(-4-((5-fluoro-4-(3-(2-oxopiperidin-1-yl)phenyl)pyrimidin-2-yl)amino)cyclohexane-1-carbonyl)piperidin-4-yl)methyl)piperidin-1-yl)phenyl)amino)piperidine-2,6-dione (Compound 414)

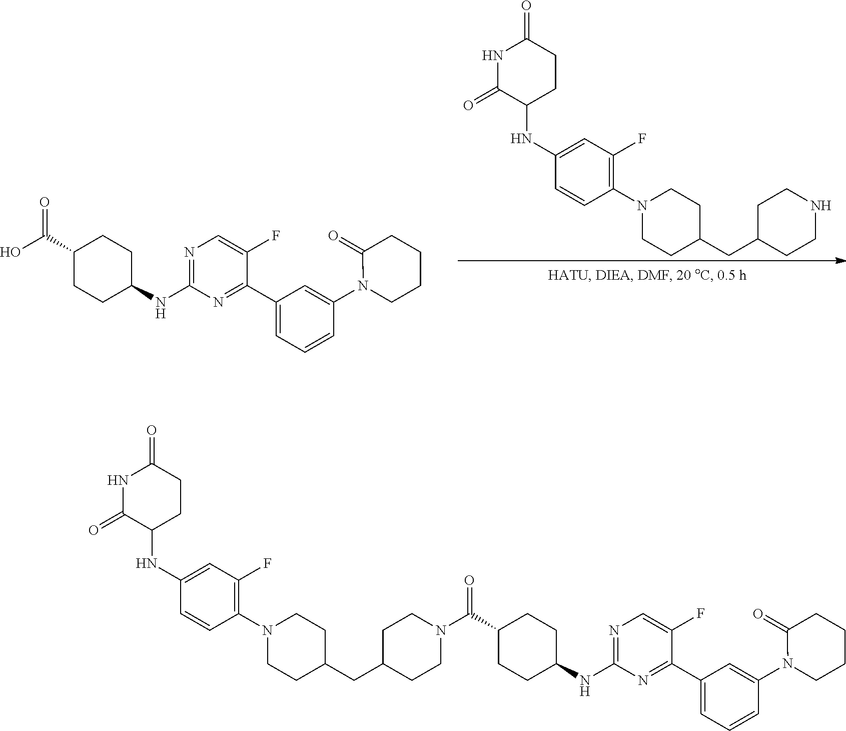 [SMILES: O=C1CCCCN1c1cccc(-c2nc(N[C@H]3CC[C@H](C(=O)O)CC3)ncc2F)c1 ; O=C1CCC(Nc2ccc(N3CCC(CC4CCNCC4)CC3)c(F)c2)C(=O)N1 ; O=C1CCC(Nc2ccc(N3CCC(CC4CCN(C(=O)[C@H]5CC[C@H](Nc6ncc(F)c(-c7cccc(N8CCCCC8=O)c7)n6)CC5)CC4)CC3)c(F)c2)C(=O)N1]

Synthesis of trans-3-((3-fluoro-4-(4-((1-(-4-((5-fluoro-4-(3-(2-oxopiperidin-1-yl)phenyl)pyrimidin-2-yl)amino)cyclohexane-1-carbonyl)piperidin-4-yl)methyl)piperidin-1-yl)phenyl)amino)piperidine-2,6-dione (Compound 414). To a solution of trans-4-((5-fluoro-4-(3-(2-oxopiperidin-1-yl)phenyl)pyrimidin-2-yl)amino)cyclohexane-1-carboxylic acid (83.8 mg, 203 μmol, 0.300 eq) in DMF (5 mL) were added HATU (77.3 mg, 203 μmol, 0.300 eq) and DIEA (438 mg, 3.40 mmol, 590 μL, 5.00 eq) at 20 °C, then 3-((3-fluoro-4-(4-(piperidin-4-ylmethyl)piperidin-1-yl)phenyl)amino)piperidine-2,6-dione (350 mg, 678 μmol, 1.00 eq, TFA) was added to the mixture, the mixture was stirred at 20 °C for 0.5 h. LCMS showed 3-((3-fluoro-4-(4-(piperidin-4-ylmethyl)piperidin-1-yl)phenyl)amino)piperidine-2,6-dione was consumed and the desired target MS was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 15%-55% B over 8.0 min) to give the title compound (Compound 414) (55.9 mg, 66.9 μmol, 9.87% yield, 95.4% purity) as a yellow solid. LCMS (ES⁺): m/z 797.4 [M+H]⁺

### Example 508. Synthesis of trans-3-[3-fluoro-4-[4-[[1-[4-[[5-fluoro-4-[3-(2-oxo-1-piperidyl)phenyl]pyrimidin-2-yl]amino]cyclohexanecarbonyl]-4-piperidyl]methyl]piperazin-1-yl]anilino]piperidine-2,6-dione (Compound 393)

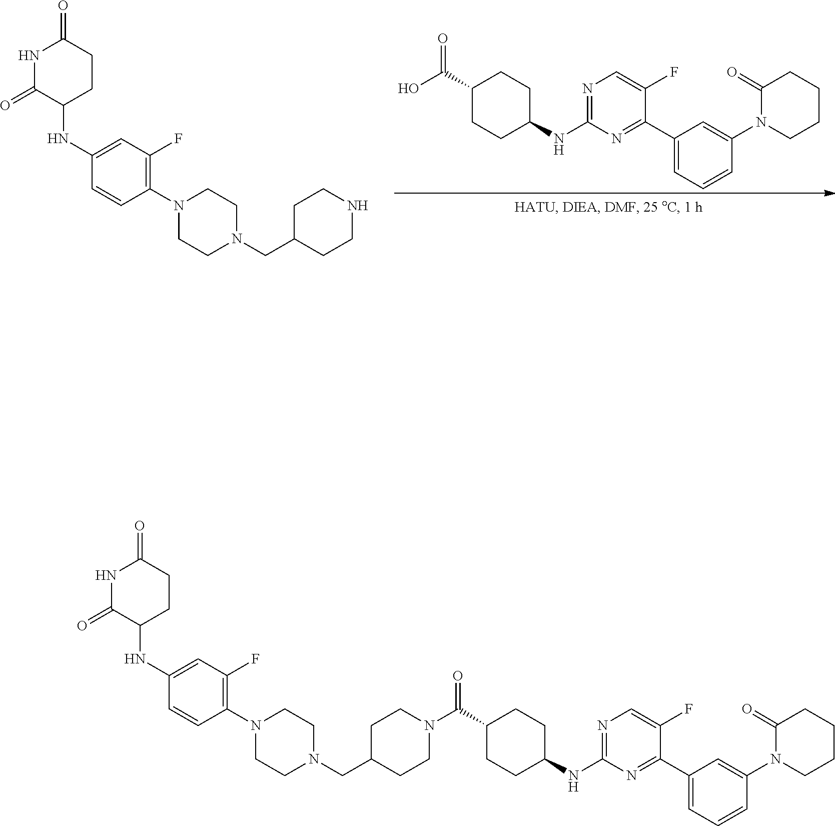 [SMILES: O=C1CCC(Nc2ccc(N3CCN(CC4CCNCC4)CC3)c(F)c2)C(=O)N1 ; O=C1CCCCN1c1cccc(-c2nc(N[C@H]3CC[C@H](C(=O)O)CC3)ncc2F)c1 ; O=C1CCC(Nc2ccc(N3CCN(CC4CCN(C(=O)[C@H]5CC[C@H](Nc6ncc(F)c(-c7cccc(N8CCCCC8=O)c7)n6)CC5)CC4)CC3)c(F)c2)C(=O)N1]

Synthesis of trans-3-[3-fluoro-4-[4-[[1-[4-[[5-fluoro-4-[3-(2-oxo-1-piperidyl)phenyl]pyrimidin-2-yl]amino]cyclohexanecarbonyl]-4-piperidyl]methyl]piperazin-1-yl]anilino]piperidine-2,6-dione (Compound 393). To a solution of trans-4-((5-fluoro-4-(3-(2-oxopiperidin-1-yl)phenyl)pyrimidin-2-yl)amino)cyclohexane-1-carboxylic acid (319 mg, 773 μmol, 1.00 eq) in DMF (4 mL) were added HATU (441 mg, 1.16 mmol, 1.50 eq) and DIEA (300 mg, 2.32 mmol, 404 μL, 3.00 eq) at 25 °C. And then 3-((3-fluoro-4-(4-(piperidin-4-ylmethyl)piperazin-1-yl)phenyl)amino)piperidine-2,6-dione (400 mg, 773 μmol, 1.00 eq, TFA) was added at 25 °C. The resulting mixture was stirred at 25 °C for 1 h. LCMS showed that 3-((3-fluoro-4-(4-(piperidin-4-ylmethyl)piperazin-1-yl)phenyl)amino)piperidine-2,6-dione was consumed completely and the desired mass was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 5%-35% B over 8.0 min) to give the title compound (Compound 393) (116 mg, 132 μmol, 17.1% yield, 90.7% purity) as a yellow solid. LCMS (ESI+): m/s 798.3 [M+H]⁺

### Example 509. Synthesis of trans-3-((3-fluoro-4-(1-((1-(4-((5-fluoro-4-(3-(2-oxopiperidin-1-yl)phenyl)pyrimidin-2-yl)amino)cyclohexane-1-carbonyl)piperidin-4-yl)methyl)piperidin-4-yl)phenyl)amino)piperidine-2,6-dione (Compound 394)

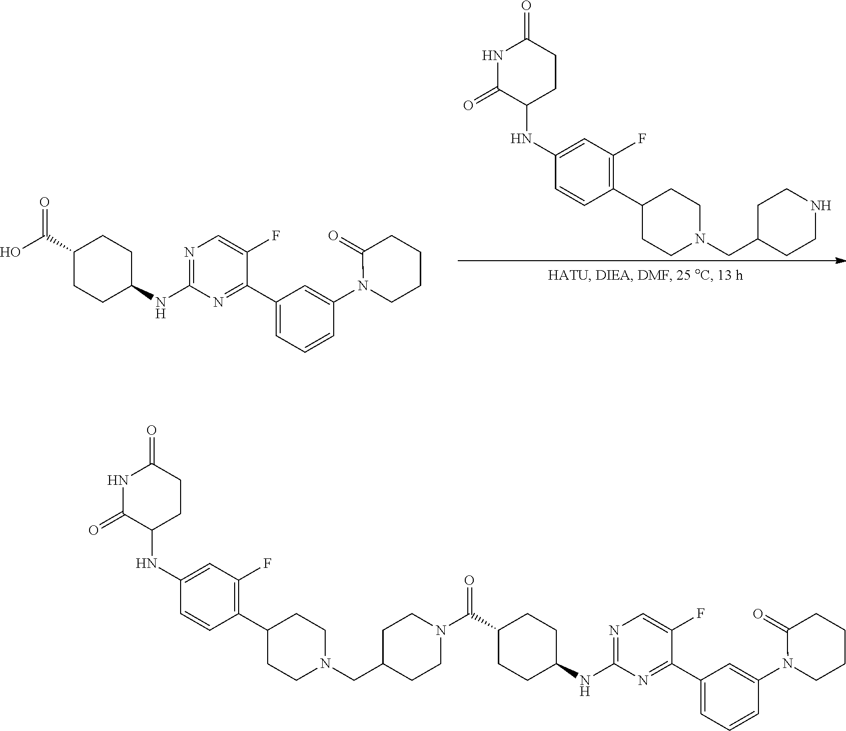 [SMILES: O=C1CCCCN1c1cccc(-c2nc(N[C@H]3CC[C@H](C(=O)O)CC3)ncc2F)c1 ; O=C1CCC(Nc2ccc(C3CCN(CC4CCNCC4)CC3)c(F)c2)C(=O)N1 ; O=C1CCC(Nc2ccc(C3CCN(CC4CCN(C(=O)[C@H]5CC[C@H](Nc6ncc(F)c(-c7cccc(N8CCCCC8=O)c7)n6)CC5)CC4)CC3)c(F)c2)C(=O)N1]

Synthesis of trans-3-((3-fluoro-4-(1-((1-(4-((5-fluoro-4-(3-(2-oxopiperidin-1-yl)phenyl)pyrimidin-2-yl)amino)cyclohexane-1-carbonyl)piperidin-4-yl)methyl)piperidin-4-yl)phenyl)amino)piperidine-2,6-dione (Compound 394). To a solution of trans-4-((5-fluoro-4-(3-(2-oxopiperidin-1-yl)phenyl)pyrimidin-2-yl)amino)cyclohexane-1-carboxylic acid (300 mg, 727 μmol, 1.00 eq) in DMF (10 mL) were added DIEA (470 mg, 3.63 mmol, 633 μL, 5.00 eq) and HATU (277 mg, 727 μmol, 1.00 eq) at 25 °C. Then to the mixture was added 3-((3-fluoro-4-(1-(piperidin-4-ylmethyl)piperidin-4-yl)phenyl)amino)piperidine-2,6-dione (413 mg, 800 μmol, 1.10 eq, TFA) at 25 °C. The mixture was stirred at 25 °C for 3 h. LCMS showed trans-4-((5-fluoro-4-(3-(2-oxopiperidin-1-yl)phenyl)pyrimidin-2-yl)amino)cyclohexane-1-carboxylic acid was consumed completely and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to give the title compound (Compound 394) (78.4 mg, 94.5 umol, 13.0% yield, 96.0% purity) as a white solid. LCMS (ES⁺): m/z 797.4 [M+H]⁺

### Example 510. Synthesis of trans-(R)-3-((3-fluoro-4-(1-((1-((1r,4r)-4-((5-fluoro-4-(3-(2-oxopiperidin-1-yl)phenyl)pyrimidin-2-yl)amino)cyclohexane-1-carbonyl)piperidin-4-yl)methyl)piperidin-4-yl)phenyl)amino)piperidine-2,6-dione (Compound 448) and trans-(S)-3-((3-fluoro-4-(1-((1-((1r,4r)-4-((5-fluoro-4-(3-(2-oxopiperidin-1-yl)phenyl)pyrimidin-2-yl)amino)cyclohexane-1-carbonyl)piperidin-4-yl)methyl)piperidin-4-yl)phenyl)amino)piperidine-2,6-dione (Compound 449) (The Two Isomers were Assigned Arbitrarily)

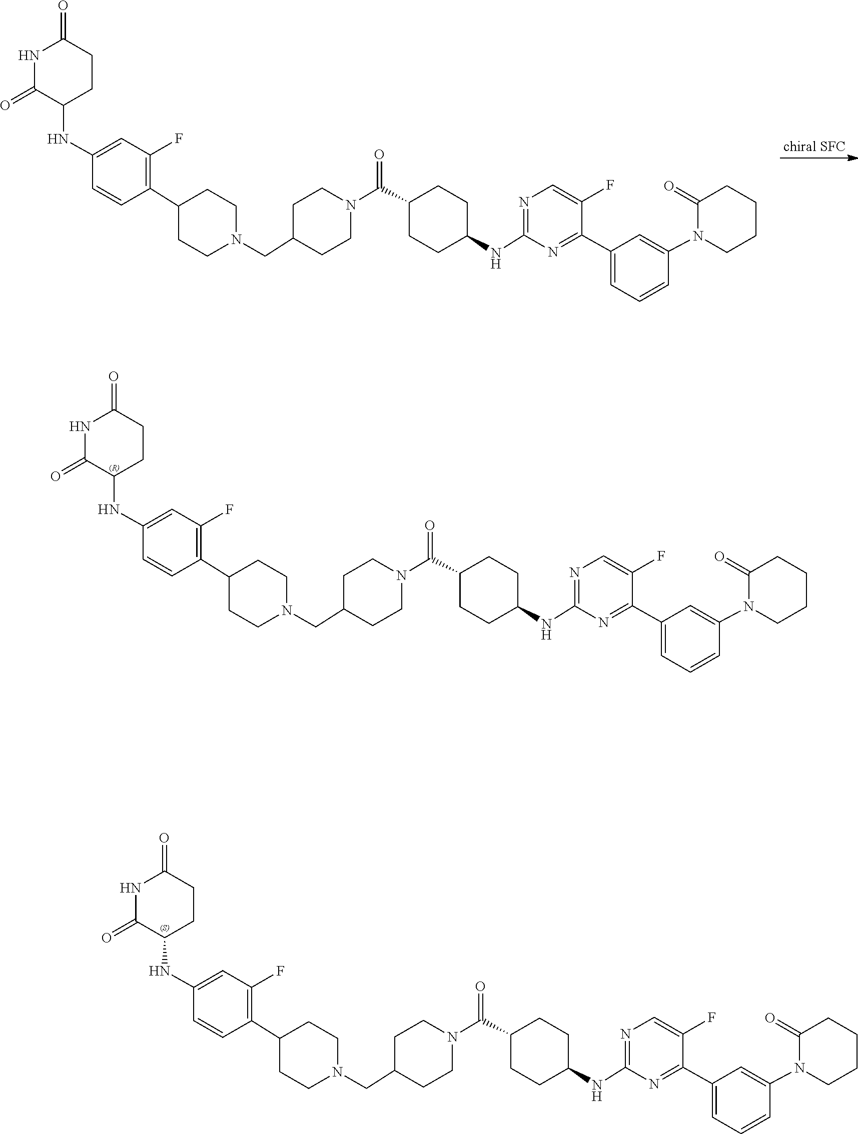

Synthesis of trans-(R)-3-((3-fluoro-4-(1-((1-((1r,4r)-4-((5-fluoro-4-(3-(2-oxopiperidin-1-yl)phenyl)pyrimidin-2-yl)amino)cyclohexane-1-carbonyl)piperidin-4-yl)methyl)piperidin-4-yl)phenyl)amino)piperidine-2,6-dione (Compound 448) and trans-(S)-3-((3-fluoro-4-(1-((1-((1r,4r)-4-((5-fluoro-4-(3-(2-oxopiperidin-1-yl)phenyl)pyrimidin-2-yl)amino)cyclohexane-1-carbonyl)piperidin-4-yl)methyl)piperidin-4-yl)phenyl)amino)piperidine-2,6-dione (Compound 449) (the two isomers were assigned arbitrarily). Trans-3-((3-fluoro-4-(1-((1-((1r,4r)-4-((5-fluoro-4-(3-(2-oxopiperidin-1-yl)phenyl)pyrimidin-2-yl)amino)cyclohexane-1-carbonyl)piperidin-4-yl)methyl)piperidin-4-yl)phenyl)amino)piperidine-2,6-dione (Compound 394) (50.0 mg, 62.7 umol, 1.00 eq) was separated by chiral SFC (column: ChiralPak IH, 250*30 mm, 10 um; mobile phase: [CO₂-IPA (0.1% IPAm)]; B %: 60%, isocratic elution mode) to give the title compound (Compound 448) (3.80 mg, 4.77 μmol, 7.60% yield, 100% purity, 100% ee) (retention time: 1.691, peak 1) as a white solid. LCMS (ES⁺): m/z 797.4 [M+H]⁺ and title compound (Compound 449) (2.80 mg, 3.40 μmol, 5.42% yield, 96.7% purity, 99.6% ee) (retention time: 3.281, peak 2) as a white solid. LCMS (ES⁺): m/z 797.4 [M+H]⁺

### Example 511. Synthesis of trans-3-((3-fluoro-4-(4-((4-(4-((5-fluoro-4-(3-(2-oxopiperidin-1-yl)phenyl)pyrimidin-2-yl)amino)cyclohexane-1-carbonyl)piperazin-1-yl)methyl)piperidin-1-yl)phenyl)amino)piperidine-2,6-dione (Compound 415)

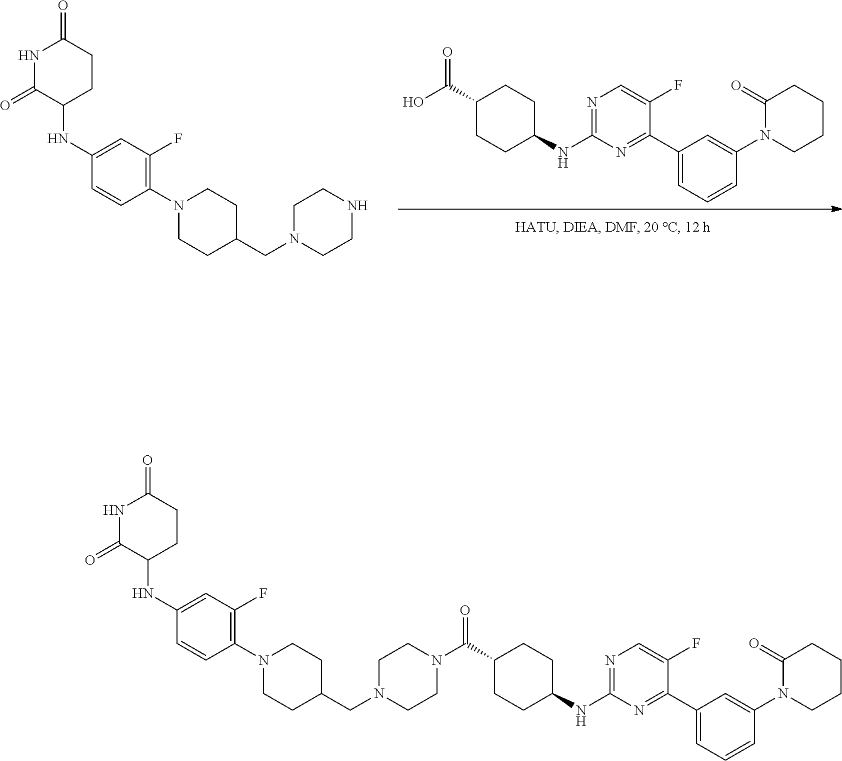 [SMILES: O=C1CCC(Nc2ccc(N3CCC(CN4CCNCC4)CC3)c(F)c2)C(=O)N1 ; O=C1CCCCN1c1cccc(-c2nc(N[C@H]3CC[C@H](C(=O)O)CC3)ncc2F)c1 ; O=C1CCC(Nc2ccc(N3CCC(CN4CCN(C(=O)[C@H]5CC[C@H](Nc6ncc(F)c(-c7cccc(N8CCCCC8=O)c7)n6)CC5)CC4)CC3)c(F)c2)C(=O)N1]

Synthesis of trans-3-((3-fluoro-4-(4-((4-(4-((5-fluoro-4-(3-(2-oxopiperidin-1-yl)phenyl)pyrimidin-2-yl)amino)cyclohexane-1-carbonyl)piperazin-1-yl)methyl)piperidin-1-yl)phenyl)amino)piperidine-2,6-dione (Compound 415). To a solution of trans-4-((5-fluoro-4-(3-(2-oxopiperidin-1-yl)phenyl)pyrimidin-2-yl)amino)cyclohexane-1-carboxylic acid (478 mg, 1.16 mmol, 0.600 eq) in DMF (10 mL) were added DIEA (1.25 g, 9.65 mmol, 1.68 mL, 5.00 eq) and HATU (735 mg, 1.93 mmol, 1.00 eq) at 20 °C. Then to the mixture was added 3-((3-fluoro-4-(4-(piperazin-1-ylmethyl)piperidin-1-yl)phenyl)amino)piperidine-2,6-dione (1.00 g, 1.93 mmol, 1.00 eq, TFA) at 20 °C. The mixture was stirred at 20 °C for 12 h. LCMS showed 3-((3-fluoro-4-(4-(piperazin-1-ylmethyl)piperidin-1-yl)phenyl)amino)piperidine-2,6-dione was consumed completely and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 25%-60% B over 8.0 min) to give the title compound (Compound 415) (195 mg, 244 umol, 12.7% yield, 100% purity) as a pink solid. LCMS (ES⁺): m/z 798.4 [M+H]⁺

### Example 512. Synthesis of trans-N-(trans-4-(4-(4-((2,6-dioxopiperidin-3-yl)amino)-2-fluorophenyl)piperidin-1-yl)cyclohexyl)-4-((5-fluoro-4-(3-(2-oxopiperidin-1-yl)phenyl)pyrimidin-2-yl)amino)cyclohexane-1-carboxamide (Compound 565)

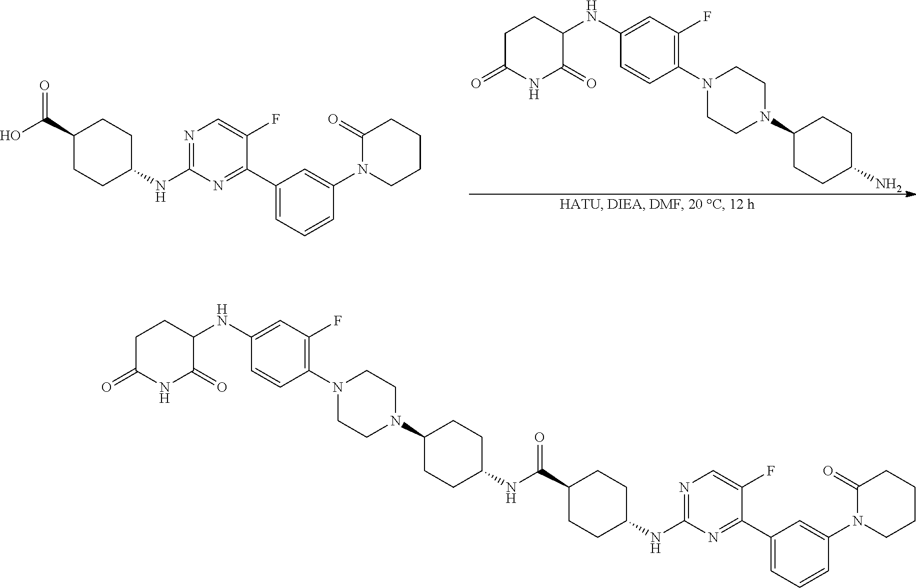 [SMILES: O=C1CCCCN1c1cccc(-c2nc(N[C@H]3CC[C@H](C(=O)O)CC3)ncc2F)c1 ; N[C@H]1CC[C@H](N2CCN(c3ccc(NC4CCC(=O)NC4=O)cc3F)CC2)CC1 ; O=C1CCC(Nc2ccc(N3CCN([C@H]4CC[C@H](NC(=O)[C@H]5CC[C@H](Nc6ncc(F)c(-c7cccc(N8CCCCC8=O)c7)n6)CC5)CC4)CC3)c(F)c2)C(=O)N1]

Synthesis of trans-N-(trans-4-(4-(4-((2,6-dioxopiperidin-3-yl)amino)-2-fluorophenyl)piperidin-1-yl)cyclohexyl)-4-((5-fluoro-4-(3-(2-oxopiperidin-1-yl)phenyl)pyrimidin-2-yl)amino)cyclohexane-1-carboxamide (Compound 565). To a solution of trans-(1r,4r)-4-((5-fluoro-4-(3-(2-oxopiperidin-1-yl)phenyl)pyrimidin-2-yl)amino)cyclohexane-1-carboxylic acid (200 mg, 484 μmol, 1.00 eq) and rac-3-((4-(1-((1r,4r)-4-aminocyclohexyl)piperidin-4-yl)-3-fluorophenyl)amino)piperidine-2,6-dione (250 mg, 484 μmol, 1.00 eq, TFA) in DMF (5 mL) were added HATU (460 mg, 1.21 mmol, 2.50 eq) and DIEA (188 mg, 1.45 mmol, 253 μL, 3.00 eq) at 20 °C. The mixture was stirred at 20 °C for 12 h. LC-MS showed that trans-(1r,4r)-4-((5-fluoro-4-(3-(2-oxopiperidin-1-yl)phenyl)pyrimidin-2-yl)amino)cyclohexane-1-carboxylic acid was completely and desired mass was detected. The residue was diluted with H₂O (10 mL) and extracted with EtOAc (5 mL*5). The combined organic layers were washed with brine (10 mL), dried over Na₂SO₄, filtered and concentrated under reduced pressure to give a residue. The residue was purified by prep-HPLC (column: 3_Phenomenex Luna C18 75*30 mm*3 um; mobile phase: [H₂O (0.2% FA)-ACN]; gradient: 5%-35% B over 8.0 min) to give the title compound (Compound 565) (77.7 mg, 95.7 μmol, 19.8% yield, 98.7% purity, 0.1 FA) as a white solid. LCMS (ES⁺): m/z 797.3 [M+H]⁺

### Example 513. Synthesis of (1r,4R)—N-((1r,4R)-4-(4-(4-((2,6-dioxopiperidin-3-yl)amino)-2-fluorophenyl)piperazin-1-yl)cyclohexyl)-4-((5-fluoro-4-(3-(2-oxopiperidin-1-yl)phenyl)pyrimidin-2-yl)amino)cyclohexane-1-carboxamide (Compound 564)

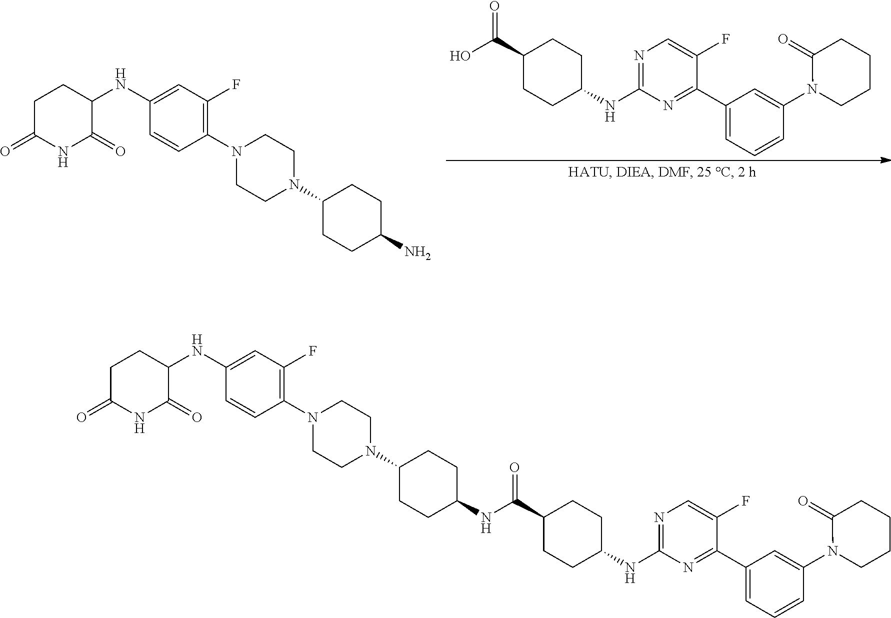 [SMILES: N[C@H]1CC[C@H](N2CCN(c3ccc(NC4CCC(=O)NC4=O)cc3F)CC2)CC1 ; O=C1CCCCN1c1cccc(-c2nc(N[C@H]3CC[C@H](C(=O)O)CC3)ncc2F)c1 ; O=C1CCC(Nc2ccc(N3CCN([C@H]4CC[C@H](NC(=O)[C@H]5CC[C@H](Nc6ncc(F)c(-c7cccc(N8CCCCC8=O)c7)n6)CC5)CC4)CC3)c(F)c2)C(=O)N1]

Synthesis of (1r,4R)—N-((1r,4R)-4-(4-(4-((2,6-dioxopiperidin-3-yl)amino)-2-fluorophenyl)piperazin-1-yl)cyclohexyl)-4-((5-fluoro-4-(3-(2-oxopiperidin-1-yl)phenyl)pyrimidin-2-yl)amino)cyclohexane-1-carboxamide (Compound 564). To a solution of rac-3-((4-(4-((1r,4r)-4-aminocyclohexyl)piperazin-1-yl)-3-fluorophenyl)amino)piperidine-2,6-dione (300 mg, 580 μmol, 1.00 eq, TFA) in DMF (5 mL) was added DIEA (225 mg, 1.74 mmol, 303 μL, 3.00 eq), rac-(1r,4r)-4-((5-fluoro-4-(3-(2-oxopiperidin-1-yl)phenyl)pyrimidin-2-yl)amino)cyclohexane-1-carboxylic acid (167 mg, 406 μmol, 0.700 eq) and HATU (331 mg, 870 μmol, 1.50 eq) at 25 °C. The mixture was stirred at 25 °C for 2 h. LC-MS showed that rac-3-((4-(4-((1r,4r)-4-aminocyclohexyl)piperazin-1-yl)-3-fluorophenyl)amino)piperidine-2,6-dione was consumed completely and the desired mass was detected. The reaction mixture was concentrated under reduced pressure. The residue was purified by prep-HPLC (column: Phenomenex luna C18 100*40 mm*5 um; mobile phase: [H₂O (0.2% FA)-ACN]; gradient: 10%-45% B over 8.0 min) to give title Compound (Compound 564) (207 mg, 242 μmol, 41.8% yield, 93.3% purity) as a pale yellow solid. LCMS (ES⁺): m/z 798.5 [M+H]⁺.

### Example 514. Synthesis of trans-3-((3-fluoro-4-(4-((1-(4-((5-fluoro-4-(3-(2-oxopiperidin-1-yl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 582)

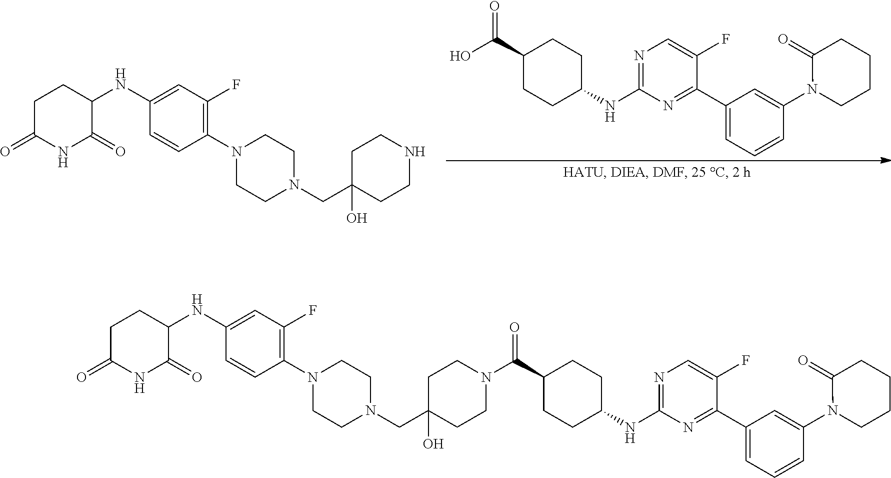 [SMILES: O=C1CCC(Nc2ccc(N3CCN(CC4(O)CCNCC4)CC3)c(F)c2)C(=O)N1 ; O=C1CCCCN1c1cccc(-c2nc(N[C@H]3CC[C@H](C(=O)O)CC3)ncc2F)c1 ; O=C1CCC(Nc2ccc(N3CCN(CC4(O)CCN(C(=O)[C@H]5CC[C@H](Nc6ncc(F)c(-c7cccc(N8CCCCC8=O)c7)n6)CC5)CC4)CC3)c(F)c2)C(=O)N1]

Synthesis of 8-acetyl-2-(2-(2-isopropylphenyl)-4-((pyridin-2-ylmethyl)amino)pyrimidin-5-yl)-2,8-diazaspiro[4.5]decan-1-one (Compound 582). To a solution of 3-((3-fluoro-4-(4-((4-hydroxypiperidin-4-yl)methyl)piperazin-1-yl)phenyl)amino)piperidine-2,6-dione (300 mg, 715 μmol, 1.00 eq) and trans-4-[[5-fluoro-4-[3-(2-oxo-1-piperidyl)phenyl]pyrimidin-2-yl]amino]cyclohexanecarboxylic acid (294 mg, 715 μmol, 1.00 eq) in DMF (3 mL) was added HATU (407 mg, 1.07 mmol, 1.50 eq) and DIEA (277 mg, 2.15 mmol, 373 μL, 3.00 eq) at 25 °C. The mixture was stirred at 25 °C for 2 h. LC-MS showed 3-((3-fluoro-4-(4-((4-hydroxypiperidin-4-yl)methyl)piperazin-1-yl)phenyl)amino)piperidine-2,6-dione was consumed completely and the desired mass was detected. The reaction mixture was filtered and concentrated under reduced pressure. The residue was purified by prep-HPLC (column: Phenomenex luna C18 100*40 mm*5 um; mobile phase: [H₂O (0.2% FA)-ACN]; gradient: 10%-50% B over 8.0 min) to give the title compound (Compound 582) (99.3 mg, 118 μmol, 16.6% yield, 97.3% purity) as a white solid. LCMS (ES⁺): m/z 814.4 [M+H]⁺.

### Example 515. Synthesis of 3-((3-fluoro-4-(1-((1-(trans-4-((5-fluoro-4-(3-(2-oxopiperidin-1-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 579)

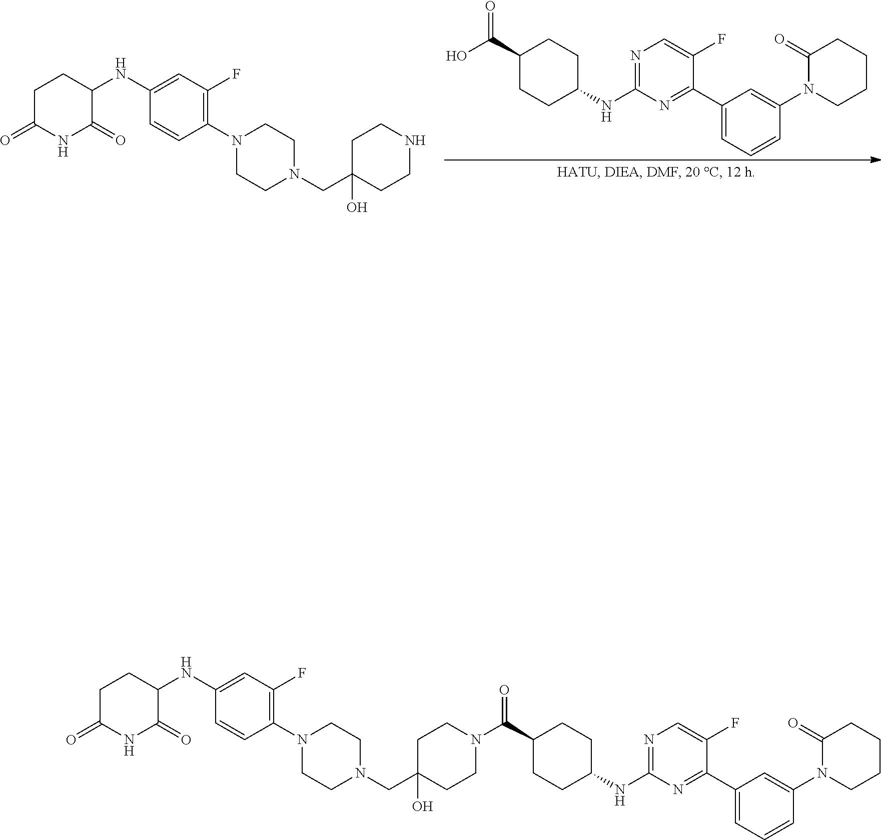 [SMILES: O=C1CCC(Nc2ccc(N3CCN(CC4(O)CCNCC4)CC3)c(F)c2)C(=O)N1 ; O=C1CCCCN1c1cccc(-c2nc(N[C@H]3CC[C@H](C(=O)O)CC3)ncc2F)c1 ; O=C1CCC(Nc2ccc(N3CCN(CC4(O)CCN(C(=O)[C@H]5CC[C@H](Nc6ncc(F)c(-c7cccc(N8CCCCC8=O)c7)n6)CC5)CC4)CC3)c(F)c2)C(=O)N1]

Synthesis of 3-((3-fluoro-4-(1-((1-(trans-4-((5-fluoro-4-(3-(2-oxopiperidin-1-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 579). To a solution of 3-((3-fluoro-4-(1-((4-hydroxypiperidin-4-yl)methyl)piperidin-4-yl)phenyl)amino)piperidine-2,6-dione (500 mg, 1.19 mmol, 1.00 eq) in DMF (5 mL) was added trans-4-((5-fluoro-4-(3-(2-oxopiperidin-1-yl)phenyl)pyrimidin-2-yl)amino)cyclohexane-1-carboxylic acid (246 mg, 597 μmol, 0.500 eq), DIEA (308 mg, 2.39 mmol, 416 μL, 2.00 eq) and HATU (908 mg, 2.39 mmol, 2.00 eq) at 20 °C. The mixture was stirred at 20 °C for 12 h. LC-MS showed the desired mass was detected. The reaction mixture was filtered and the filtrate concentrated under high vacuum. The residue was purified by prep-HPLC (Waters Xbridge Prep OBD C18 150*40 mm*Oum; mobile phase: [H₂O (10 mM NH₄HCO₃)-ACN]; gradient: 30%-70% B over 8.0 min) to give the title compound (Compound 579) (77.8 mg, 93.4 μmol, 7.82% yield, 97.6% purity) as a pale yellow solid. LCMS (ES⁺): m/z 813.4 [M+H]⁺.

### Example 516. Synthesis of trans-(1r,4R)—N-(((1r,4R)-4-((4-(4-((2,6-dioxopiperidin-3-yl)amino)-2-fluorophenyl)piperazin-1-yl)methyl)cyclohexyl)methyl)-4-((5-fluoro-4-(3-(2-oxopiperidin-1-yl)phenyl)pyrimidin-2-yl)amino)cyclohexane-1-carboxamide (Compound 590)

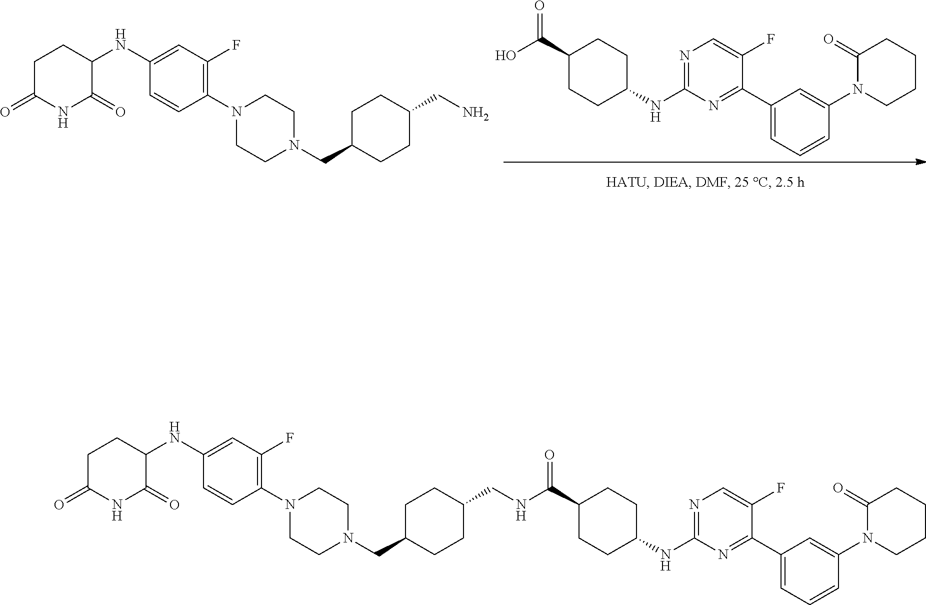 [SMILES: NC[C@H]1CC[C@H](CN2CCN(c3ccc(NC4CCC(=O)NC4=O)cc3F)CC2)CC1 ; O=C1CCCCN1c1cccc(-c2nc(N[C@H]3CC[C@H](C(=O)O)CC3)ncc2F)c1 ; O=C1CCC(Nc2ccc(N3CCN(C[C@H]4CC[C@H](CNC(=O)[C@H]5CC[C@H](Nc6ncc(F)c(-c7cccc(N8CCCCC8=O)c7)n6)CC5)CC4)CC3)c(F)c2)C(=O)N1]

Synthesis of trans-(1r,4R)—N-(((1r,4R)-4-((4-(4-((2,6-dioxopiperidin-3-yl)amino)-2-fluorophenyl)piperazin-1-yl)methyl)cyclohexyl)methyl)-4-((5-fluoro-4-(3-(2-oxopiperidin-1-yl)phenyl)pyrimidin-2-yl)amino)cyclohexane-1-carboxamide (Compound 590). To a solution of trans-4-((5-fluoro-4-(3-(2-oxopiperidin-1-yl)phenyl)pyrimidin-2-yl)amino)cyclohexane-1-carboxylic acid (700 mg, 1.70 mmol, 1.00 eq) in DMF (20 mL) were added DIEA (1.10 g, 8.48 mmol, 1.48 mL, 5.00 eq) and HATU (645 mg, 1.70 mmol, 1.00 eq) at 25 °C. Then to the mixture was added trans-3-((4-(4-((4-(aminomethyl)cyclohexyl)methyl)piperazin-1-yl)-3-fluorophenyl)amino)piperidine-2,6-dione (926 mg, 1.70 mmol, 1.00 eq, TFA) at 25 °C. The mixture was stirred at 25 °C for 2.5 h. LCMS showed trans-3-((4 (4-((4-(aminomethyl)cyclohexyl)methyl)piperazin-1-yl)-3-fluorophenyl)amino)piperidine-2,6-dione was consumed and the desired mass was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 25%-55% B over 8.0 min) to give the title compound (Compound 590) (498 mg, 603 μmol, 35.5% yield, 100% purity) as a white solid. LCMS (ES⁺): m/z 826.5 [M+H]⁺

### Example 517. Synthesis of trans-N-(1′-(4-((2,6-dioxopiperidin-3-yl)amino)-2-fluorophenyl)-3′,3′-difluoro-[1,4′-bipiperidin]-4-yl)-4-((5-fluoro-4-(3-(2-oxopiperidin-1-yl)phenyl)pyrimidin-2-yl)amino)cyclohexane-1-carboxamide (Compound 494)

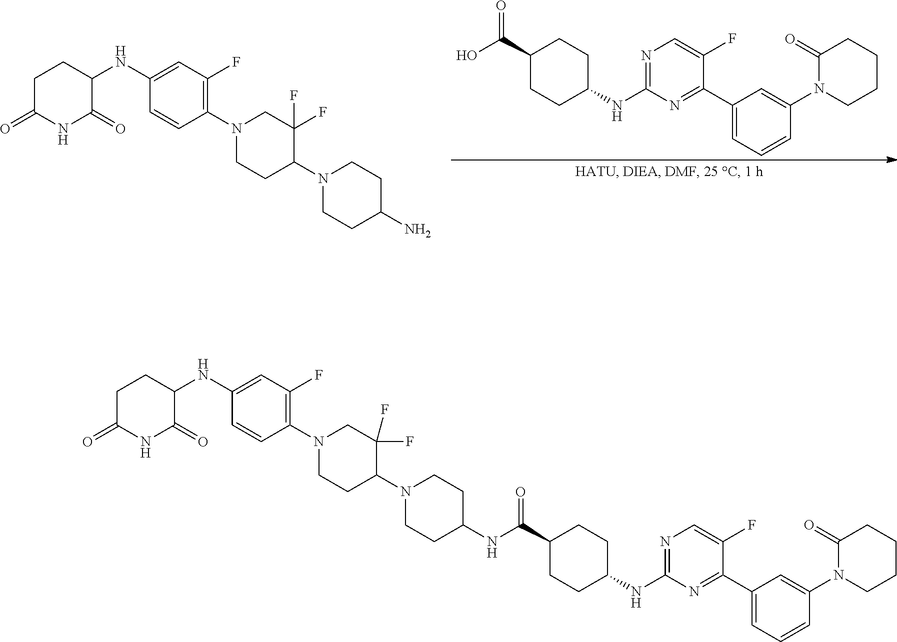 [SMILES: NC1CCN(C2CCN(c3ccc(NC4CCC(=O)NC4=O)cc3F)CC2(F)F)CC1 ; O=C1CCCCN1c1cccc(-c2nc(N[C@H]3CC[C@H](C(=O)O)CC3)ncc2F)c1 ; O=C1CCC(Nc2ccc(N3CCC(N4CCC(NC(=O)[C@H]5CC[C@H](Nc6ncc(F)c(-c7cccc(N8CCCCC8=O)c7)n6)CC5)CC4)C(F)(F)C3)c(F)c2)C(=O)N1]

Synthesis of trans-N-(1′-(4-((2,6-dioxopiperidin-3-yl)amino)-2-fluorophenyl)-3′,3′-difluoro-[1,4′-bipiperidin]-4-yl)-4-((5-fluoro-4-(3-(2-oxopiperidin-1-yl)phenyl)pyrimidin-2-yl)amino)cyclohexane-1-carboxamide (Compound 494). To a solution of trans-4-((5-fluoro-4-(3-(2-oxopiperidin-1-yl)phenyl)pyrimidin-2-yl)amino)cyclohexane-1-carboxylic acid (74.5 mg, 181 μmol, 0.500 eq) in DMF (7 mL) were added HATU (48.1 mg, 126 μmol, 0.350 eq), DIEA (234 mg, 1.81 mmol, 315 μL, 5.00 eq) and 3-((4-(4-amino-3′,3′-difluoro-[1,4′-bipiperidin]-1′-yl)-3-fluorophenyl)amino)piperidine-2,6-dione (200 mg, 361 μmol, 1.00 eq, TFA) at 25 °C. The mixture was stirred at 25 °C for 1 h. LCMS showed 3-((4-(4-amino-3′,3′-difluoro-[1,4′-bipiperidin]-1′-yl)-3-fluorophenyl)amino)piperidine-2,6-dione was consumed completely and the desired mass was detected. The reaction mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-70% B over 8.0 min) to give the title compound (Compound 494) (71.3 mg, 81.0 μmol, 22.4% yield, 94.8% purity) as a white solid. LCMS (ES⁺): m/z 834.4 [M+H]⁺.

### Example 518. Synthesis of trans-N-(1′-(4-((2,6-dioxopiperidin-3-yl)amino)-2-fluorophenyl)-[1,4′-bipiperidin]-4-yl)-4-((5-fluoro-4-(3-(2-oxopiperidin-1-yl)phenyl)pyrimidin-2-yl)amino)cyclohexane-1-carboxamide (Compound 521)

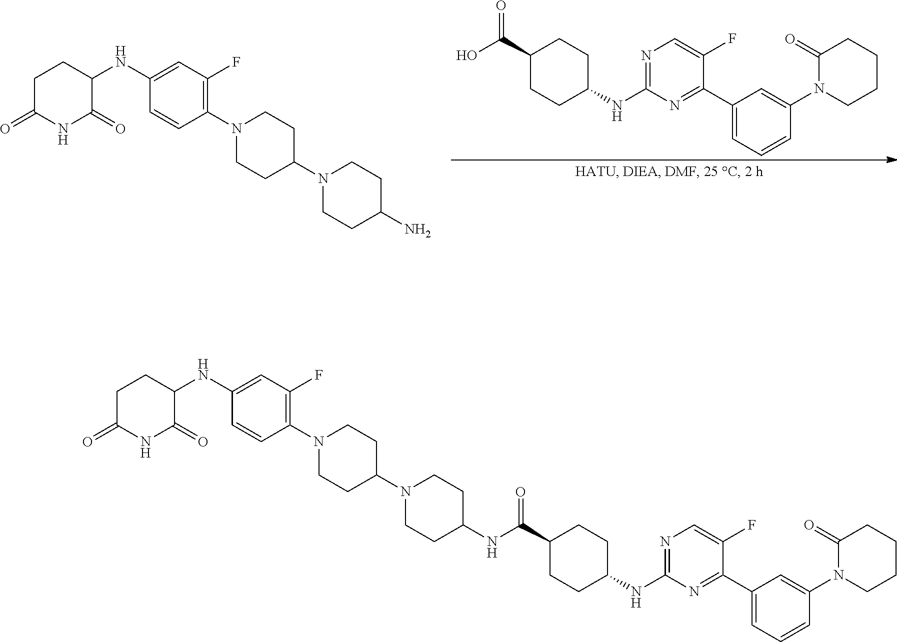 [SMILES: NC1CCN(C2CCN(c3ccc(NC4CCC(=O)NC4=O)cc3F)CC2)CC1 ; O=C1CCCCN1c1cccc(-c2nc(N[C@H]3CC[C@H](C(=O)O)CC3)ncc2F)c1 ; O=C1CCC(Nc2ccc(N3CCC(N4CCC(NC(=O)[C@H]5CC[C@H](Nc6ncc(F)c(-c7cccc(N8CCCCC8=O)c7)n6)CC5)CC4)CC3)c(F)c2)C(=O)N1]

Synthesis of trans-N-(1′-(4-((2,6-dioxopiperidin-3-yl)amino)-2-fluorophenyl)-[1,4′-bipiperidin]-4-yl)-4-((5-fluoro-4-(3-(2-oxopiperidin-1-yl)phenyl)pyrimidin-2-yl)amino)cyclohexane-1-carboxamide (Compound 521). To a solution of trans-4-((5-fluoro-4-(3-(2-oxopiperidin-1-yl)phenyl)pyrimidin-2-yl)amino)cyclohexane-1-carboxylic acid (98.2 mg, 238 μmol, 0.400 eq) in DMF (3 mL) were added DIEA (385 mg, 2.98 mmol, 518 μL, 5.00 eq) and HATU (79.2 mg, 208 μmol, 0.350 eq) at 25 °C. Then to the mixture was added 3-((4-(4-amino-[1,4′-bipiperidin]-1′-yl)-3-fluorophenyl)amino)piperidine-2,6-dione (308 mg, 595 μmol, 1.00 eq, TFA) at 25 °C. The mixture was stirred at 25 °C for 2 h. LCMS showed 3-((4-(4-amino-[1,4′-bipiperidin]-1′-yl)-3-fluorophenyl)amino)piperidine-2,6-dione was consumed and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to give the title compound (Compound 521) (30.3 mg, 35.5 μmol, 5.96% yield, 93.5% purity) as a pale yellow solid. LCMS (ES⁺): m/z 798.4 [M+H]⁺.

### Example 519. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(2-oxo-2H-[1,2′-bipyridin]-4′-yl)pyrimidin-2-yl)amino)cyclohexyl)cyclohexane-1-carboxamide (Compound 573)

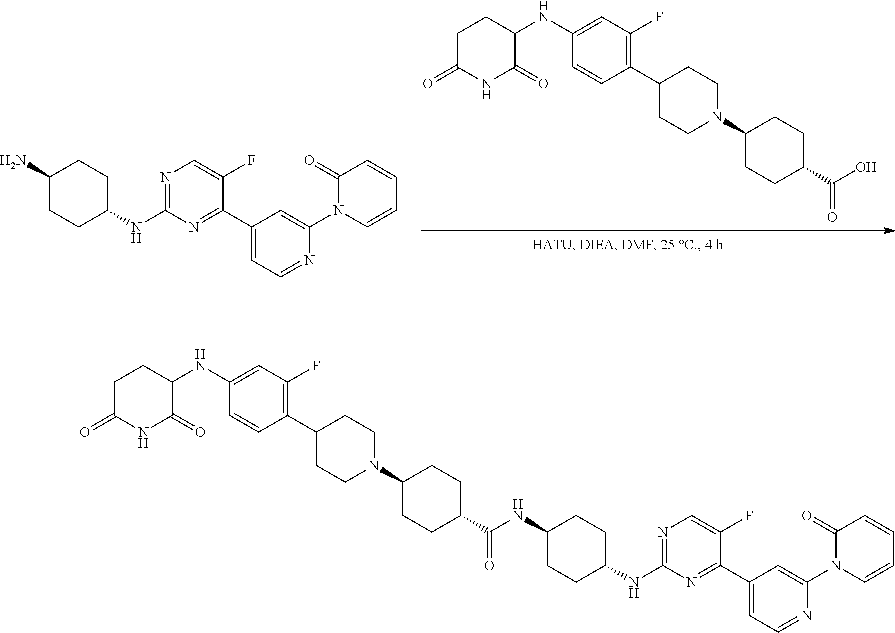 [SMILES: N[C@H]1CC[C@H](Nc2ncc(F)c(-c3ccnc(-n4ccccc4=O)c3)n2)CC1 ; O=C1CCC(Nc2ccc(C3CCN([C@H]4CC[C@H](C(=O)O)CC4)CC3)c(F)c2)C(=O)N1 ; O=C1CCC(Nc2ccc(C3CCN([C@H]4CC[C@H](C(=O)N[C@H]5CC[C@H](Nc6ncc(F)c(-c7ccnc(-n8ccccc8=O)c7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(2-oxo-2H-[1,2′-bipyridin]-4′-yl)pyrimidin-2-yl)amino)cyclohexyl)cyclohexane-1-carboxamide (Compound 573). To a solution of trans-4-(4-(4-((2,6-dioxopiperidin-3-yl)amino)-2-fluorophenyl)piperidin-1-yl)cyclohexane-1-carboxylic acid (266 mg, 616 μmol, 1.00 eq) in DMF (2 mL) were added DIEA (398 mg, 3.08 mmol, 538 μL, 5.00 eq) and HATU (93.8 mg, 247 μmol, 0.400 eq) at 25 °C. Then the mixture was added trans-4′-(2-((4-aminocyclohexyl)amino)-5-fluoropyrimidin-4-yl)-2H-[1,2′-bipyridin]-2-one (152 mg, 308 μmol, 0.500 eq, TFA) at 25 °C. The mixture was stirred at 25 °C for 4 h. LCMS showed trans-4′-(2-((4-aminocyclohexyl)amino)-5-fluoropyrimidin-4-yl)-2H-[1,2′-bipyridin]-2-one was consumed and the desired mass was detected. The mixture was filtered and the filtrate was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 15%-55% B over 8.0 min) to give the title compound (Compound 573) (94.2 mg, 115 μmol, 18.7% yield, 96.94% purity) as a pale yellow solid. LCMS (ES⁺): m/z 794.4 [M+H]⁺

### Example 520. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(3-(5-methyl-2-oxopyridin-1 (2H)-yl)phenyl)pyrimidin-2-yl)amino)cyclohexyl)cyclohexane-1-carboxamide (Compound

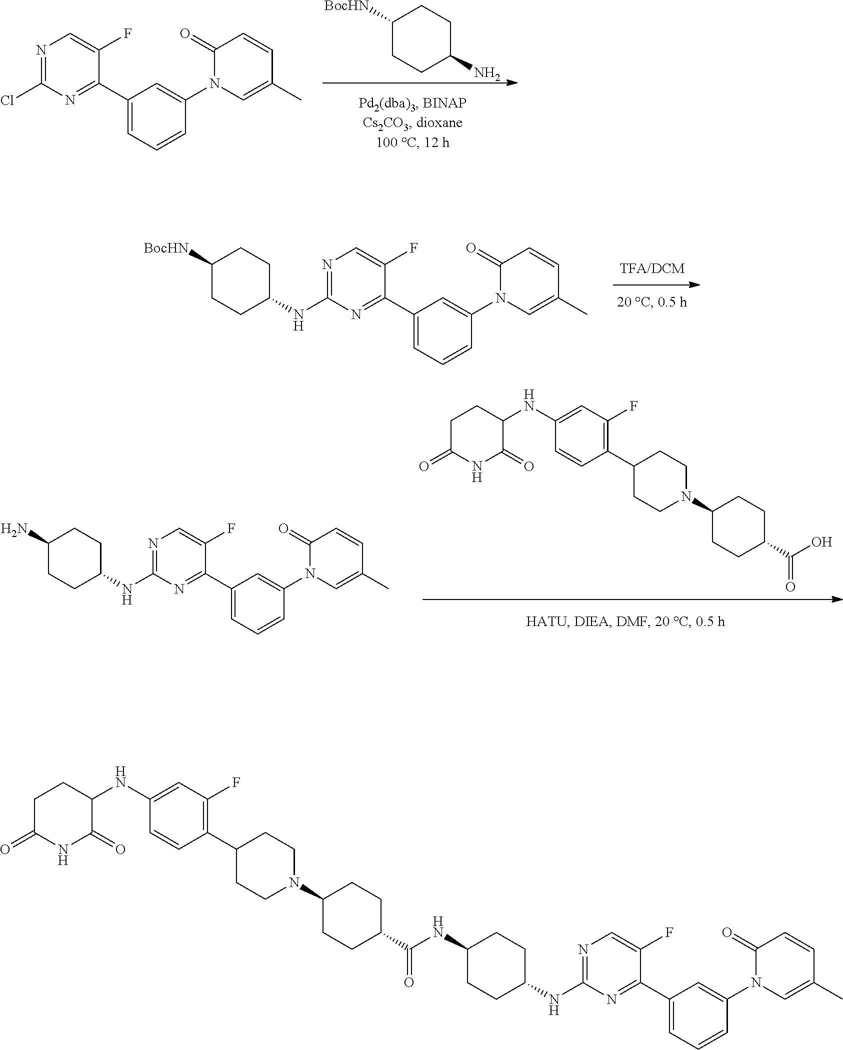 [SMILES: Cc1ccc(=O)n(-c2cccc(-c3nc(Cl)ncc3F)c2)c1 ; CC(C)(C)OC(=O)N[C@H]1CC[C@H](N)CC1 ; Cc1ccc(=O)n(-c2cccc(-c3nc(N[C@H]4CC[C@H](NC(=O)OC(C)(C)C)CC4)ncc3F)c2)c1 ; Cc1ccc(=O)n(-c2cccc(-c3nc(N[C@H]4CC[C@H](N)CC4)ncc3F)c2)c1 ; O=C1CCC(Nc2ccc(C3CCN([C@H]4CC[C@H](C(=O)O)CC4)CC3)c(F)c2)C(=O)N1 ; Cc1ccc(=O)n(-c2cccc(-c3nc(N[C@H]4CC[C@H](NC(=O)[C@H]5CC[C@H](N6CCC(c7ccc(NC8CCC(=O)NC8=O)cc7F)CC6)CC5)CC4)ncc3F)c2)c1]

Synthesis of trans-tert-butyl (4-((5-fluoro-4-(3-(5-methyl-2-oxopyridin-1 (2H)-yl)phenyl)pyrimidin-2-yl)amino)cyclohexyl)carbamate. To a solution of 1-(3-(2-chloro-5-fluoropyrimidin-4-yl)phenyl)-5-methylpyridin-2 (1H)-one (3.50 g, 11.1 mmol, 1.00 eq) and trans-tert-butyl (4-aminocyclohexyl)carbamate (3.56 g, 16.6 mmol, 1.50 eq) in dioxane (150 mL) were added BINAP (621 mg, 998 μmol, 0.0900 eq), Pd₂(dba)₃ (508 mg, 554 μmol, 0.0500 eq) and Cs₂CO₃ (10.8 g, 33.3 mmol, 3.00 eq) at 20 °C under N₂ atmosphere. The mixture was stirred at 100 °C for 12 h under N₂ atmosphere. LCMS showed 1-(3-(2-chloro-5-fluoropyrimidin-4-yl)phenyl)-5-methylpyridin-2 (1H)-one was consumed and the desired target MS was detected. H₂O (100 mL) was added to the mixture and the mixture was extracted with EtOAc (100 mL*3), the combined organic layers were washed with brine (200 mL), dried over Na₂SO₄, filtered and the filtrate was concentrated under reduced pressure to give a residue. The residue was purified by MPLC (SiO₂, PE/EtOAc=1/0 to 0/1) to give the title compound (3.80 g, 7.70 mmol, 69.5% yield) as a yellow solid. LCMS (ES⁺): m/z 494.2 [M+H]⁺

Synthesis of trans-1-(3-(2-((4-aminocyclohexyl)amino)-5-fluoropyrimidin-4-yl)phenyl)-5-methylpyridin-2 (1H)-one. A solution of trans-tert-butyl (4-((5-fluoro-4-(3-(5-methyl-2-oxopyridin-1 (2H)-yl)phenyl)pyrimidin-2-yl)amino)cyclohexyl)carbamate (85.0 mg, 172 μmol, 1.00 eq) in DCM (3 mL) and TFA (1 mL) was stirred at 20 °C for 0.5 h. LCMS showed trans-tert-butyl (4-((5-fluoro-4-(3-(5-methyl-2-oxopyridin-1 (2H)-yl)phenyl)pyrimidin-2-yl)amino)cyclohexyl)carbamate was consumed and the desired target MS was detected. The mixture was concentrated under reduced pressure to give the title compound (87.0 mg, crude, TFA) as a brown oil. LCMS (ES⁺): m/z 394.2 [M+H]⁺

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(3-(5-methyl-2-oxopyridin-1 (2H)-yl)phenyl)pyrimidin-2-yl)amino)cyclohexyl)cyclohexane-1-carboxamide (Compound 574). To a solution of trans-4-(4-(4-((2,6-dioxopiperidin-3-yl)amino)-2-fluorophenyl)piperidin-1-yl)cyclohexane-1-carboxylic acid (176 mg, 408 μmol, 1.00 eq) and trans-1-(3-(2-((4-aminocyclohexyl)amino)-5-fluoropyrimidin-4-yl)phenyl)-5-methylpyridin-2 (1H)-one (82.8 mg, 163 μmol, 0.400 eq, TFA) in DMF (4 mL) were added HATU (54.3 mg, 143 μmol, 0.35 eq) and DIEA (158 mg, 1.22 mmol, 213 μL, 3.00 eq) at 20 °C, then the mixture was stirred at 20 °C for 0.5 h. LCMS showed trans-4-(4-(4-((2,6-dioxopiperidin-3-yl)amino)-2-fluorophenyl)piperidin-1-yl)cyclohexane-1-carboxylic acid was consumed and the desired target MS was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 45%-75% B over 8.0 min) to give the title compound (Compound 574) (45.3 mg, 52.7 μmol, 12.9% yield, 93.8% purity) as a white solid. LCMS (ES⁺): m/z 807.4 [M+H]⁺

### Example 521. Synthesis of trans-N-(trans-4-(4-(4-((2,6-dioxopiperidin-3-yl)amino)-2-fluorophenyl)piperidin-1-yl)cyclohexyl)-4-((5-fluoro-4-(3-(5-methyl-2-oxopyridin-1 (2H)-yl)phenyl)pyrimidin-2-yl)amino)cyclohexane-1-carboxamide (Compound 551)

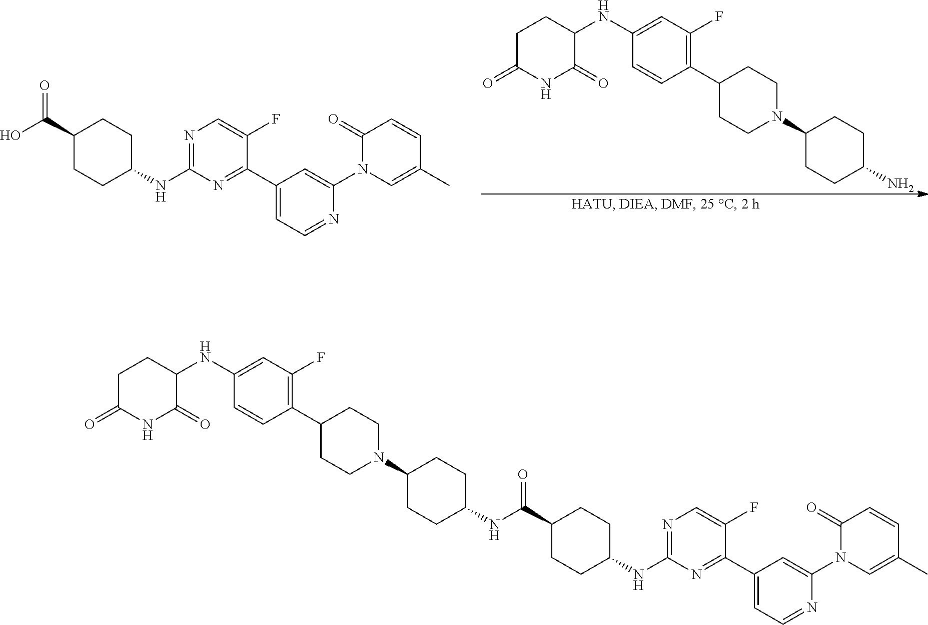 [SMILES: Cc1ccc(=O)n(-c2cc(-c3nc(N[C@H]4CC[C@H](C(=O)O)CC4)ncc3F)ccn2)c1 ; N[C@H]1CC[C@H](N2CCC(c3ccc(NC4CCC(=O)NC4=O)cc3F)CC2)CC1 ; Cc1ccc(=O)n(-c2cc(-c3nc(N[C@H]4CC[C@H](C(=O)N[C@H]5CC[C@H](N6CCC(c7ccc(NC8CCC(=O)NC8=O)cc7F)CC6)CC5)CC4)ncc3F)ccn2)c1]

Synthesis of trans-N-(trans-4-(4-(4-((2,6-dioxopiperidin-3-yl)amino)-2-fluorophenyl)piperidin-1-yl)cyclohexyl)-4-((5-fluoro-4-(3-(5-methyl-2-oxopyridin-1 (2H)-yl)phenyl)pyrimidin-2-yl)amino)cyclohexane-1-carboxamide (Compound 551). To a solution of trans-3-((4-(1-(4-aminocyclohexyl)piperidin-4-yl)-3-fluorophenyl)amino)piperidine-2,6-dione (300 mg, 580 μmol, 1.00 eq, TFA) in DMF (1.5 mL) was added DIEA (225 mg, 1.74 mmol, 303 μL, 3.00 eq) until pH-7. To a solution of trans-4-((5-fluoro-4-(3-(5-methyl-2-oxopyridin-1 (2H)-yl)phenyl)pyrimidin-2-yl)amino)cyclohexane-1-carboxylic acid (245 mg, 580 μmol, 1.00 eq) in DMF (1.5 mL) was added DIEA (225 mg, 1.74 mmol, 303 μL, 3.00 eq) until pH-7. Then HATU (331 mg, 871 μmol, 1.50 eq) was added to the mixture of the two solutions. The mixture was stirred at 25 °C for 2 h. LC-MS showed the desired mass was detected. The reaction mixture was filtered to give a residue. The residue was purified by prep-HPLC (column: Waters Xbridge BEH C18 100*30 mm*10 um; mobile phase: [H₂O (10 mM NH₄HCO₃)-ACN]; gradient: 30%-60% B over 8.0 min) to give the title compound (Compound 551) (109 mg, 130 μmol, 22.5% yield, 96.1% purity) as a pale yellow solid. LCMS (ES⁺): m/z 807.4 [M+H]⁺.

### Example 522. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(2-(2-oxo-1,3-oxazinan-3-yl)pyridin-4-yl)pyrimidin-2-yl)amino)cyclohexyl)cyclohexane-1-carboxamide (Compound 572)

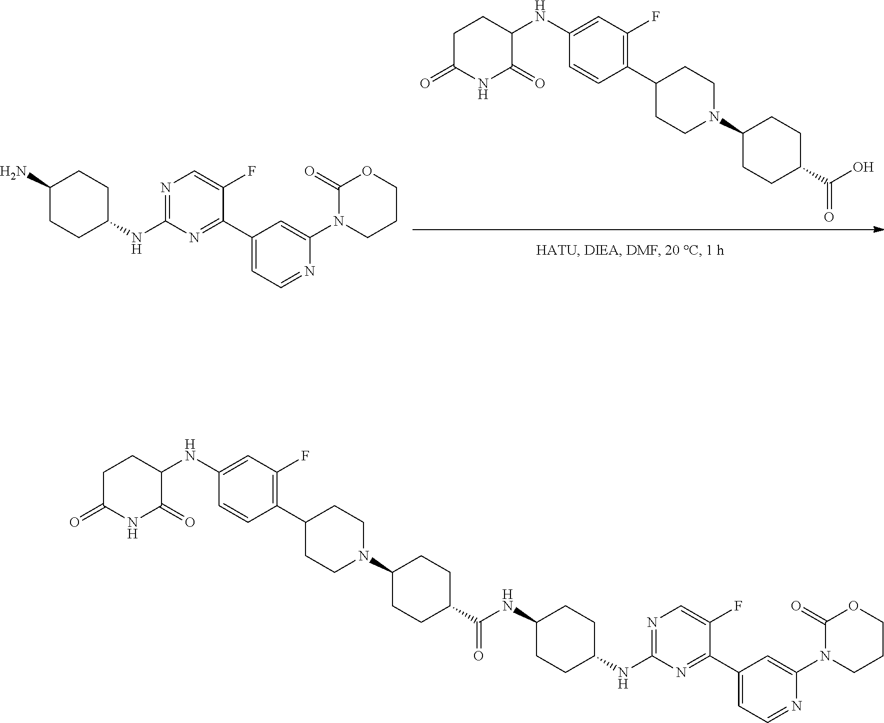 [SMILES: N[C@H]1CC[C@H](Nc2ncc(F)c(-c3ccnc(N4CCCOC4=O)c3)n2)CC1 ; O=C1CCC(Nc2ccc(C3CCN([C@H]4CC[C@H](C(=O)O)CC4)CC3)c(F)c2)C(=O)N1 ; O=C1CCC(Nc2ccc(C3CCN([C@H]4CC[C@H](C(=O)N[C@H]5CC[C@H](Nc6ncc(F)c(-c7ccnc(N8CCCOC8=O)c7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(2-(2-oxo-1,3-oxazinan-3-yl)pyridin-4-yl)pyrimidin-2-yl)amino)cyclohexyl)cyclohexane-1-carboxamide (Compound 572). To a solution of trans-4-(4-(4-((2,6-dioxopiperidin-3-yl)amino)-2-fluorophenyl)piperidin-1-yl)cyclohexane-1-carboxylic acid (250 mg, 579 μmol, 1.00 eq) in DMF (5 mL) were added HATU (77.1 mg, 203 μmol, 0.350 eq) and DIEA (225 mg, 1.74 mmol, 303 μL, 3.00 eq) at 20 °C, then trans-3-(4-(2-((4-aminocyclohexyl)amino)-5-fluoropyrimidin-4-yl)pyridin-2-yl)-1,3-oxazinan-2-one (116 mg, 232 μmol, 0.400 eq, TFA) was added to the mixture, the mixture was stirred at 20 °C for 1 h. LCMS showed trans-4-(4-(4-((2,6-dioxopiperidin-3-yl)amino)-2-fluorophenyl)piperidin-1-yl)cyclohexane-1-carboxylic acid was consumed and the desired target MS was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 20%-50% B over 8.0 min) to give the title compound (Compound 572) (76.3 mg, 88.8 μmol, 15.3% yield, 93.1% purity) as a white solid. LCMS (ES⁺): m/z 800.4 [M+H]⁺

### Example 523. Synthesis of trans-N-((trans)-4-(4-(4-((2,6-dioxopiperidin-3-yl)amino)-2-fluorophenyl)piperidin-1-yl)cyclohexyl)-4-((5-fluoro-4-(2-(2-oxo-1,3-oxazinan-3-yl)pyridin-4-yl)pyrimidin-2-yl)amino)cyclohexane-1-carboxamide (Compound 550)

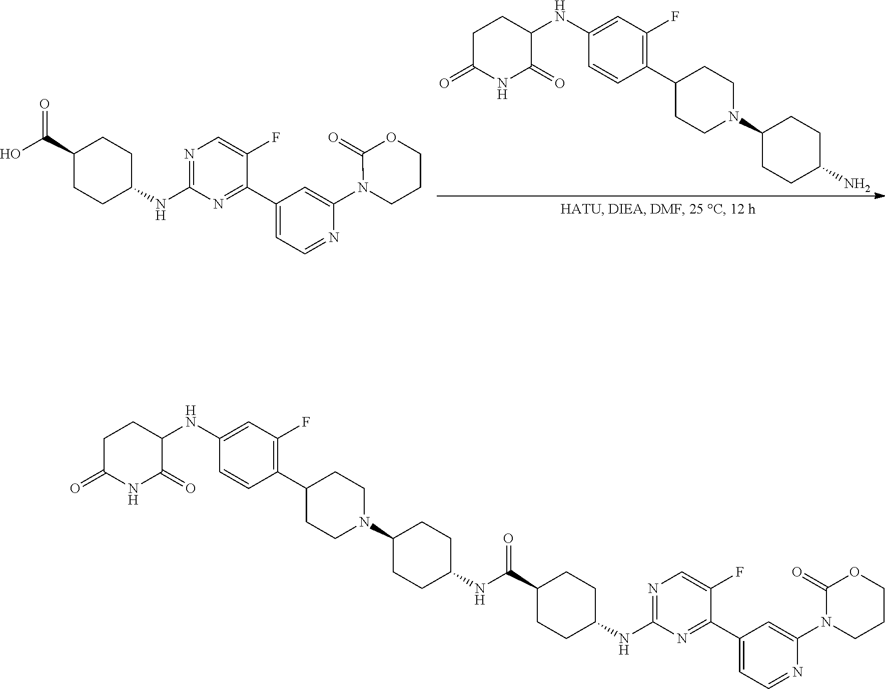 [SMILES: O=C1OCCCN1c1cc(-c2nc(N[C@H]3CC[C@H](C(=O)O)CC3)ncc2F)ccn1 ; N[C@H]1CC[C@H](N2CCC(c3ccc(NC4CCC(=O)NC4=O)cc3F)CC2)CC1 ; O=C1CCC(Nc2ccc(C3CCN([C@H]4CC[C@H](NC(=O)[C@H]5CC[C@H](Nc6ncc(F)c(-c7ccnc(N8CCCOC8=O)c7)n6)CC5)CC4)CC3)c(F)c2)C(=O)N1]

Synthesis of trans-N-((trans)-4-(4-(4-((2,6-dioxopiperidin-3-yl)amino)-2-fluorophenyl)piperidin-1-yl)cyclohexyl)-4-((5-fluoro-4-(2-(2-oxo-1,3-oxazinan-3-yl)pyridin-4-yl)pyrimidin-2-yl)amino)cyclohexane-1-carboxamide (Compound 550). To a solution of trans-4-((5-fluoro-4-(2-(2-oxo-1,3-oxazinan-3-yl)pyridin-4-yl)pyrimidin-2-yl)amino)cyclohexane-1-carboxylic acid (300 mg, 722 μmol, 1.00 eq) in DMF (4 mL) was added HATU (274 mg, 722 μmol, 1.00 eq), trans-3-((4-(1-(4-aminocyclohexyl)piperidin-4-yl)-3-fluorophenyl)amino)piperidine-2,6-dione (290 mg, 562 μmol, 7.79 eq, TFA) and DIEA (280 mg, 2.17 mmol, 377 μL, 3.00 eq) at 25 °C. The mixture was stirred at 25 °C for 12 h. LC-MS showed trans-4-((5-fluoro-4-(2-(2-oxo-1,3-oxazinan-3-yl)pyridin-4-yl)pyrimidin-2-yl)amino)cyclohexane-1-carboxylic acid was consumed completely and the desired mass was detected. The reaction mixture was concentrated under reduced pressure. The residue was purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min) to give product compound The residue was purified by prep-HPLC (column: Waters Xbridge BEH C18 100*30 mm*10 um; mobile phase: [H₂O (10 mM NH₄HCO₃)-ACN]; gradient: 20%-50% B over 8.0 min) to give the title compound (Compound 550) (33.9 mg, 41.9 μmol, 5.80% yield, 100% purity, 0.200 FA) as a white solid. LCMS (ES⁺): m/z 800.4 [M+H]⁺.

### Example 524. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(2-(2-oxopiperidin-1-yl)pyridin-4-yl)pyrimidin-2-yl)amino)cyclohexyl)cyclohexane-1-carboxamide (Compound 571)

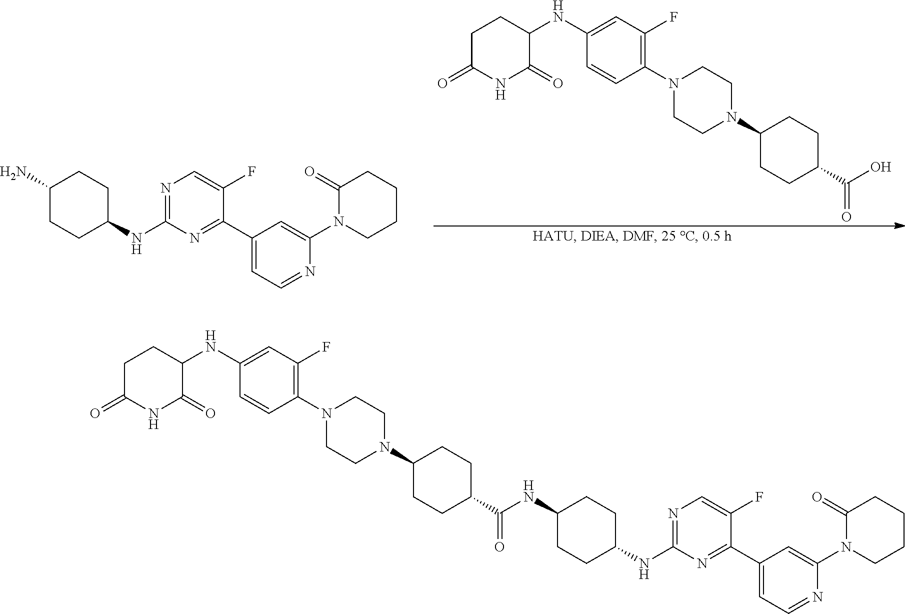 [SMILES: N[C@H]1CC[C@H](Nc2ncc(F)c(-c3ccnc(N4CCCCC4=O)c3)n2)CC1 ; O=C1CCC(Nc2ccc(N3CCN([C@H]4CC[C@H](C(=O)O)CC4)CC3)c(F)c2)C(=O)N1 ; O=C1CCC(Nc2ccc(N3CCN([C@H]4CC[C@H](C(=O)N[C@H]5CC[C@H](Nc6ncc(F)c(-c7ccnc(N8CCCCC8=O)c7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(2-(2-oxopiperidin-1-yl)pyridin-4-yl)pyrimidin-2-yl)amino)cyclohexyl)cyclohexane-1-carboxamide (Compound 571). To a solution of trans-4-(4-(4-((2,6-dioxopiperidin-3-yl)amino)-2-fluorophenyl)piperidin-1-yl)cyclohexane-1-carboxylic acid (265 mg, 615 μmol, 1.00 eq) in DMF (5 mL) were added HATU (81.9 mg, 215 μmol, 0.350 eq), DIEA (398 mg, 3.08 mmol, 535 μL, 5.00 eq) and trans-1-(4-(2-((4-aminocyclohexyl)amino)-5-fluoropyrimidin-4-yl)pyridin-2-yl)piperidin-2-one (123 mg, 246 μmol, 0.400 eq, TFA) at 25 °C. The mixture was stirred at 25 °C for 0.5 h. LCMS showed trans-1-(4-(2-((4-aminocyclohexyl)amino)-5-fluoropyrimidin-4-yl)pyridin-2-yl)piperidin-2-one was consumed completely and the desired mass was detected. The reaction mixture was concentrated under reduced pressure to remove solvent. The residue was purified by prep-HPLC (column: Phenomenex Luna C18 75*30 mm*3 um; mobile phase: [H₂O (0.2% FA)-ACN]; gradient: 10%-50% B over 8.0 min) to give the title compound (Compound 571) (64.3 mg, 74.8 μmol, 12.2% yield, 98.2% purity, FA) as a white solid. LCMS (ES⁺): m/z 798.5 [M+H]⁺

### Example 525. Synthesis of trans-N-(trans-4-(4-(4-((2,6-dioxopiperidin-3-yl)amino)-2-fluorophenyl)piperidin-1-yl)cyclohexyl)-4-((5-fluoro-4-(2-(2-oxopiperidin-1-yl)pyridin-4-yl)pyrimidin-2-yl)amino)cyclohexane-1-carboxamide (Compound 549)

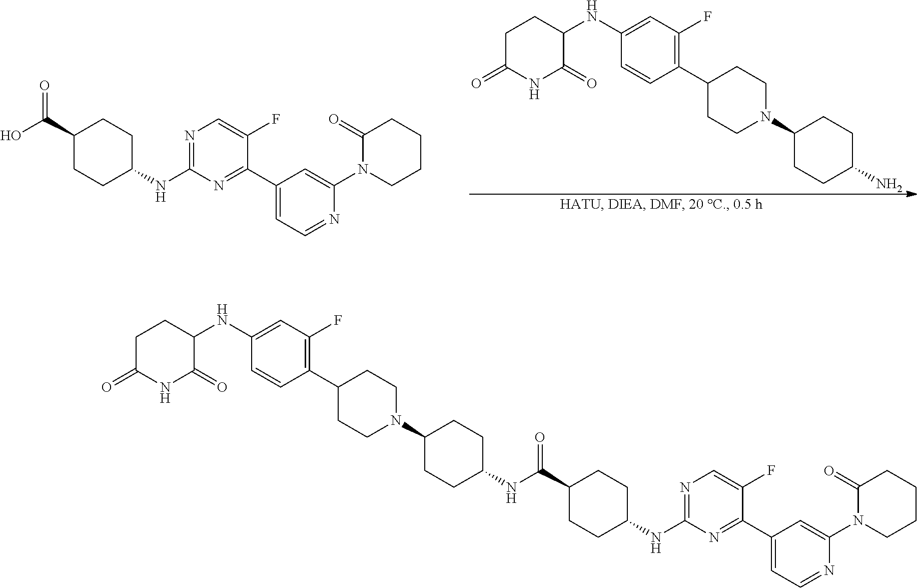 [SMILES: O=C1CCCCN1c1cc(-c2nc(N[C@H]3CC[C@H](C(=O)O)CC3)ncc2F)ccn1 ; N[C@H]1CC[C@H](N2CCC(c3ccc(NC4CCC(=O)NC4=O)cc3F)CC2)CC1 ; O=C1CCC(Nc2ccc(C3CCN([C@H]4CC[C@H](NC(=O)[C@H]5CC[C@H](Nc6ncc(F)c(-c7ccnc(N8CCCCC8=O)c7)n6)CC5)CC4)CC3)c(F)c2)C(=O)N1]

Synthesis of trans-N-(trans-4-(4-(4-((2,6-dioxopiperidin-3-yl)amino)-2-fluorophenyl)piperidin-1-yl)cyclohexyl)-4-((5-fluoro-4-(2-(2-oxopiperidin-1-yl)pyridin-4-yl)pyrimidin-2-yl)amino)cyclohexane-1-carboxamide (Compound 549). To a solution of trans-4-[[5-fluoro-4-[2-(2-oxo-1-piperidyl)-4-pyridyl]pyrimidin-2-yl]amino]cyclohexanecarboxylic acid (144 mg, 348 μmol, 0.600 eq) in DMF (5 mL) was added trans-3-[4-[1-(4-aminocyclohexyl)-4-piperidyl]-3-fluoro-anilino]piperidine-2,6-dione (300 mg, 580 μmol, 1.00 eq, TFA), DIEA (225 mg, 1.74 mmol, 303.50 μL, 3.00 eq) and HATU (331 mg, 871 μmol, 1.50 eq) at 20 °C. The mixture was stirred at 20 °C for 0.5 h. LC-MS showed the desired mass was detected. The reaction mixture was concentrated under reduced pressure. The residue was purified by prep-HPLC (column: Phenomenex Luna C18 100*30 mm*3 um; mobile phase: [H₂O (0.2% FA)-ACN]; gradient: 10%-40% B over 8.0 min) to give the title compound (Compound 549) (136 mg, 163 μmol, 28.1% yield, 95.4% purity) as a yellow solid. LCMS (ES⁺): m/z 798.4 [M+H]⁺.

### Example 526. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(2-(3-oxomorpholino)pyridin-4-yl)pyrimidin-2-yl)amino)cyclohexyl)cyclohexane-1-carboxamide (Compound 570)

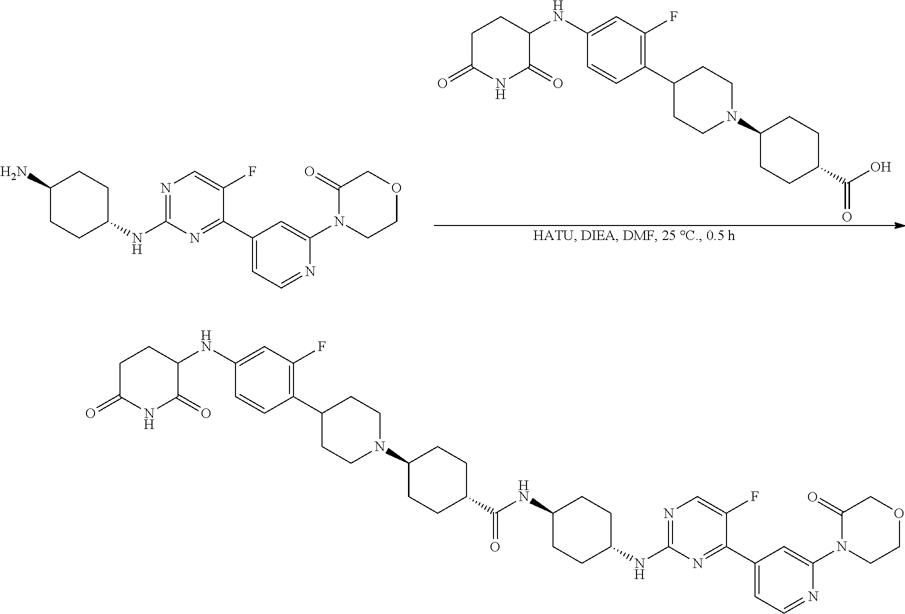 [SMILES: N[C@H]1CC[C@H](Nc2ncc(F)c(-c3ccnc(N4CCOCC4=O)c3)n2)CC1 ; O=C1CCC(Nc2ccc(C3CCN([C@H]4CC[C@H](C(=O)O)CC4)CC3)c(F)c2)C(=O)N1 ; O=C1CCC(Nc2ccc(C3CCN([C@H]4CC[C@H](C(=O)N[C@H]5CC[C@H](Nc6ncc(F)c(-c7ccnc(N8CCOCC8=O)c7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(2-(3-oxomorpholino)pyridin-4-yl)pyrimidin-2-yl)amino)cyclohexyl)cyclohexane-1-carboxamide (Compound 570). To a solution of trans-4-(4-(4-((2,6-dioxopiperidin-3-yl)amino)-2-fluorophenyl)piperidin-1-yl)cyclohexane-1-carboxylic acid (265 mg, 615 μmol, 1.00 eq) in DMF (5 mL) were added HATU (81.9 mg, 215 μmol, 0.350 eq), DIEA (159 mg, 1.23 mmol, 214 μL, 2.00 eq) and trans-4-(4-(2-((4-aminocyclohexyl)amino)-5-fluoropyrimidin-4-yl)pyridin-2-yl)morpholin-3-one (123 mg, 246 μmol, 0.400 eq, TFA) at 25 °C. The mixture was stirred at 25 °C for 0.5 h. LCMS showed trans-4-(4-(2-((4-aminocyclohexyl)amino)-5-fluoropyrimidin-4-yl)pyridin-2-yl)morpholin-3-one was consumed completely and the desired mass was detected. The reaction mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-55% B over 8.0 min) to give the title compound (Compound 570) (56.7 mg, 67.3 μmol, 10.9% yield, 95.0% purity) as a white solid. LCMS (ES⁺): m/z 800.4 [M+H]⁺.

### Example 527. Synthesis of trans-N-((trans)-4-(4-(4-((2,6-dioxopiperidin-3-yl)amino)-2-fluorophenyl)piperidin-1-yl)cyclohexyl)-4-((5-fluoro-4-(2-(3-oxomorpholino)pyridin-4-yl)pyrimidin-2-yl)amino)cyclohexane-1-carboxamide (Compound 480)

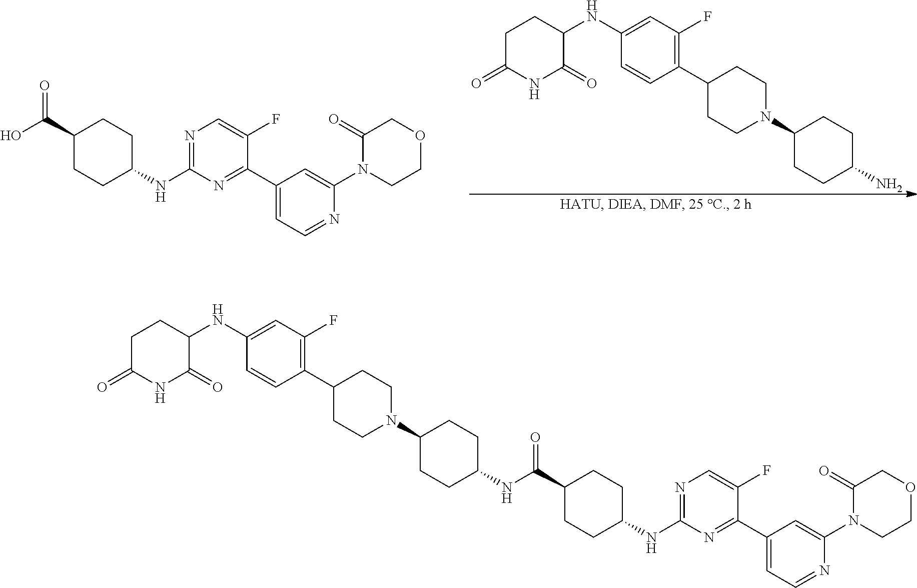 [SMILES: O=C1COCCN1c1cc(-c2nc(N[C@H]3CC[C@H](C(=O)O)CC3)ncc2F)ccn1 ; N[C@H]1CC[C@H](N2CCC(c3ccc(NC4CCC(=O)NC4=O)cc3F)CC2)CC1 ; O=C1CCC(Nc2ccc(C3CCN([C@H]4CC[C@H](NC(=O)[C@H]5CC[C@H](Nc6ncc(F)c(-c7ccnc(N8CCOCC8=O)c7)n6)CC5)CC4)CC3)c(F)c2)C(=O)N1]

Synthesis of trans-N-((trans)-4-(4-(4-((2,6-dioxopiperidin-3-yl)amino)-2-fluorophenyl)piperidin-1-yl)cyclohexyl)-4-((5-fluoro-4-(2-(3-oxomorpholino)pyridin-4-yl)pyrimidin-2-yl)amino)cyclohexane-1-carboxamide (Compound 480). To a solution of trans-4-((5-fluoro-4-(2-(3-oxomorpholino)pyridin-4-yl)pyrimidin-2-yl)amino)cyclohexane-1-carboxylic acid (200 mg, 377 μmol, 1.00 eq, TFA) in DMF (2 mL) was added HATU (143 mg, 377 μmol, 1.00 eq), 3-[4-[1-(4-aminocyclohexyl)-4-piperidyl]-3-fluoro-anilino]piperidine-2,6-dione (152 mg, 294 μmol, 7.79 eq, TFA) and DIEA (146 mg, 1.13 mmol, 197 μL, 3.00 eq) at 25 °C. The mixture was stirred at 25 °C for 2 h. LC-MS showed trans-4-((5-fluoro-4-(2-(3-oxomorpholino)pyridin-4-yl)pyrimidin-2-yl)amino)cyclohexane-1-carboxylic acid was consumed completely and the desired mass was detected. The reaction mixture was concentrated under reduced pressure to give a residue. The residue was purified by prep-HPLC (column: Phenomenex Luna C18 75*30 mm*3 um; mobile phase: [water (0.04% HCl)-ACN]; gradient: 10%-45% B over 8.0 min) to give desired compound as a pale yellow solid. The residue was purified by prep-HPLC (column: Waters Xbridge BEH C18 100*30 mm*10 um; mobile phase: [H₂O (10 mM NH₄HCO₃)-ACN]; gradient: 25%-55% B over 8.0 min) to give the title compound (Compound 480) (42.1 mg, 50.5 μmol, 13.3% yield, 95.9% purity) as a pale yellow solid. LCMS (ES⁺): m/z 800.4 [M+H]⁺.

### Example 528. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(2-(1-hydroxycyclobutyl)pyridin-4-yl)pyrimidin-2-yl)amino)cyclohexyl)cyclohexane-1-carboxamide (Compound 569)

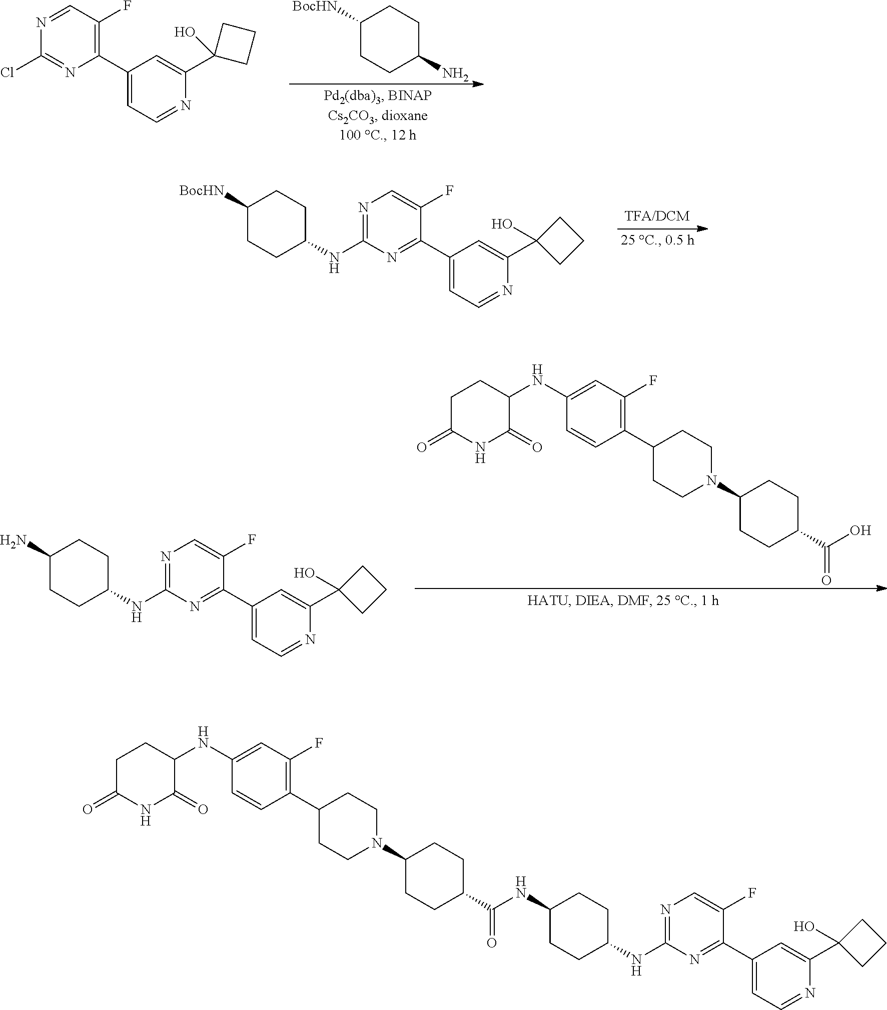 [SMILES: OC1(c2cc(-c3nc(Cl)ncc3F)ccn2)CCC1 ; CC(C)(C)OC(=O)N[C@H]1CC[C@H](N)CC1 ; CC(C)(C)OC(=O)N[C@H]1CC[C@H](Nc2ncc(F)c(-c3ccnc(C4(O)CCC4)c3)n2)CC1 ; N[C@H]1CC[C@H](Nc2ncc(F)c(-c3ccnc(C4(O)CCC4)c3)n2)CC1 ; O=C1CCC(Nc2ccc(C3CCN([C@H]4CC[C@H](C(=O)O)CC4)CC3)c(F)c2)C(=O)N1 ; O=C1CCC(Nc2ccc(C3CCN([C@H]4CC[C@H](C(=O)N[C@H]5CC[C@H](Nc6ncc(F)c(-c7ccnc(C8(O)CCC8)c7)n6)CC5)CC4)CC3)c(F)c2)C(=O)N1]

Synthesis trans-tert-butyl (4-((5-fluoro-4-(2-(1-hydroxycyclobutyl)pyridin-4-yl)pyrimidin-2-yl)amino)cyclohexyl)carbamate. To a solution of 1-(4-(2-chloro-5-fluoropyrimidin-4-yl)pyridin-2-yl)cyclobutan-1-ol (2.00 g, 7.15 mmol, 1.00 eq) in dioxane (80 mL) were added trans-tert-butyl (4-aminocyclohexyl)carbamate (1.53 g, 7.15 mmol, 1.00 eq), Cs₂CO₃ (3.49 g, 10.7 mmol, 1.50 eq), Pd₂(dba)₃ (327 mg, 358 μmol, 0.0500 eq) and BINAP (223 mg, 358 μmol, 0.0500 eq) at 25 °C under N₂ atmosphere. The mixture was stirred at 100 °C for 12 h under N₂ atmosphere. LC-MS showed 1-(4-(2-chloro-5-fluoropyrimidin-4-yl)pyridin-2-yl)cyclobutan-1-ol was consumed completely and the desired mass was detected. Water (50 mL) was added to the mixture at 25 °C. Then the reaction mixture was extracted with EtOAc (30 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=2:1 to 1:1) to give the title Compound (1.20 g, 2.62 mmol, 36.7% yield) as a yellow solid. LCMS (ES⁺): m/z 458.3 [M+H]⁺.

Synthesis of trans-1-(4-(2-((4-aminocyclohexyl)amino)-5-fluoropyrimidin-4-yl)pyridin-2-yl)cyclobutan-1-ol. To a solution of trans-tert-butyl (4-((5-fluoro-4-(2-(1-hydroxycyclobutyl)pyridin-4-yl)pyrimidin-2-yl)amino)cyclohexyl)carbamate (145 mg, 317 μmol, 1.00 eq) in DCM (3 mL) was added TFA (1 mL) at 25 °C. The mixture was stirred at 25 °C for 0.5 h. LC-MS showed trans-tert-butyl (4-((5-fluoro-4-(2-(1-hydroxycyclobutyl)pyridin-4-yl)pyrimidin-2-yl)amino)cyclohexyl)carbamate was consumed completely and the desired mass was detected. The reaction mixture was concentrated under reduced pressure to remove solvent to give the title Compound (149 mg, crude, TFA) as a yellow oil. LCMS (ES⁺): m/z 358.2 [M+H]⁺.

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(2-(1-hydroxycyclobutyl)pyridin-4-yl)pyrimidin-2-yl)amino)cyclohexyl)cyclohexane-1-carboxamide (Compound 569). To a solution of trans-1-(4-(2-((4-aminocyclohexyl)amino)-5-fluoropyrimidin-4-yl)pyridin-2-yl)cyclobutan-1-ol (145 mg, 307 μmol, 0.500 eq, TFA) in DMF (6 mL) were added DIEA (238 mg, 1.84 mmol, 321 μL, 3.00 eq), HATU (93.4 mg, 246 μmol, 0.400 eq) and trans-4-(4-(4-((2,6-dioxopiperidin-3-yl)amino)-2-fluorophenyl)piperidin-1-yl)cyclohexane-1-carboxylic acid (265 mg, 614 μmol, 1.00 eq) at 25 °C. The mixture was stirred at 25 °C for 1 h. LC-MS showed trans-1-(4-(2-((4-aminocyclohexyl)amino)-5-fluoropyrimidin-4-yl)pyridin-2-yl)cyclobutan-1-ol was consumed completely and the desired mass was detected. The reaction mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to give the title compound (Compound 569) (64.5 mg, 83.6 μmol, 13.6% yield, 99.9% purity) as a white solid. LCMS (ES⁺): m/z 771.5 [M+H]⁺

### Example 529. Synthesis of trans-N-(trans-4-(4-(4-((2,6-dioxopiperidin-3-yl)amino)-2-fluorophenyl)piperidin-1-yl)cyclohexyl)-4-((5-fluoro-4-(2-(1-hydroxycyclobutyl)pyridin-4-yl)pyrimidin-2-yl)amino)cyclohexane-1-carboxamide (Compound 548)

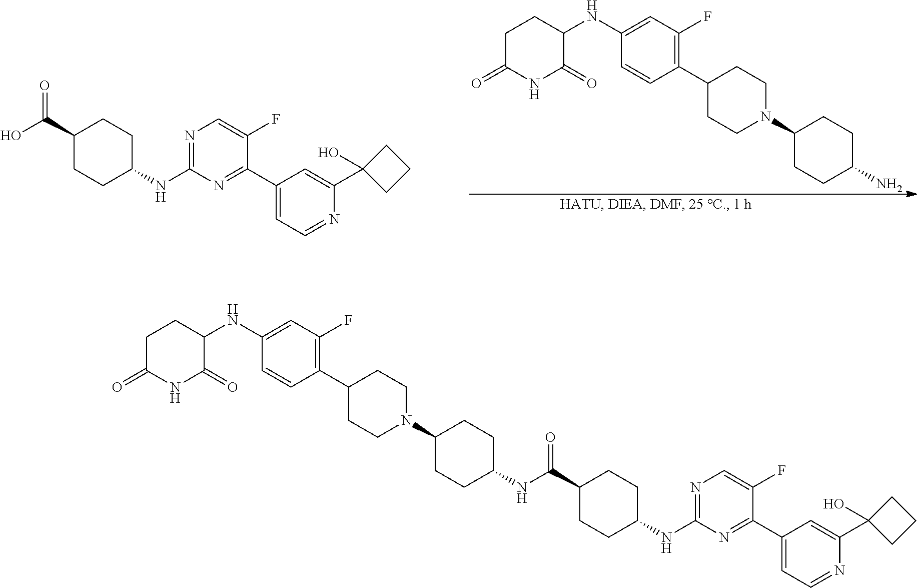 [SMILES: O=C(O)[C@H]1CC[C@H](Nc2ncc(F)c(-c3ccnc(C4(O)CCC4)c3)n2)CC1 ; N[C@H]1CC[C@H](N2CCC(c3ccc(NC4CCC(=O)NC4=O)cc3F)CC2)CC1 ; O=C1CCC(Nc2ccc(C3CCN([C@H]4CC[C@H](NC(=O)[C@H]5CC[C@H](Nc6ncc(F)c(-c7ccnc(C8(O)CCC8)c7)n6)CC5)CC4)CC3)c(F)c2)C(=O)N1]

Synthesis of trans-N-(trans-4-(4-(4-((2,6-dioxopiperidin-3-yl)amino)-2-fluorophenyl)piperidin-1-yl)cyclohexyl)-4-((5-fluoro-4-(2-(1-hydroxycyclobutyl)pyridin-4-yl)pyrimidin-2-yl)amino)cyclohexane-1-carboxamide (Compound 548). To a solution of trans-4-((5-fluoro-4-(2-(1-hydroxycyclobutyl)pyridin-4-yl)pyrimidin-2-yl)amino)cyclohexane-1-carboxylic acid (400 mg, 1.04 mmol, 1.00 eq) in DMF (1 mL) was added trans-3-((4-(1-(4-aminocyclohexyl)piperidin-4-yl)-3-fluorophenyl)amino)piperidine-2,6-dione (458 mg, 1.14 mmol, 1.10 eq), HATU (787 mg, 2.07 mmol, 2.00 eq) and DIEA (267 mg, 2.07 mmol, 360 μL, 2.00 eq). The mixture was stirred at 25 °C for 1 h. LC-MS showed the desired mass was detected. The reaction mixture was quenched by addition H₂O (3 mL), and then extracted with EtOAc (3 mL*3). The combined organic layers were dried over Na₂SO₄, filtered and concentrated under reduced pressure. The residue was purified by prep-HPLC (column: Phenomenex luna C18 100*40 mm*3 um; mobile phase: [H₂O (0.2% FA)-ACN]; gradient: 5%-40% B over 8.0 min) to give the title compound (Compound 548) (81.6 mg, 103 μmol, 10.0% yield, 97.9% purity) as a white solid. LCMS (ES⁺): m/z 771.4 [M+H]⁺

### Example 530. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(3-(3-oxomorpholino)phenyl)pyrimidin-2-yl)amino)cyclohexyl)cyclohexane-1-carboxamide (Compound 568)

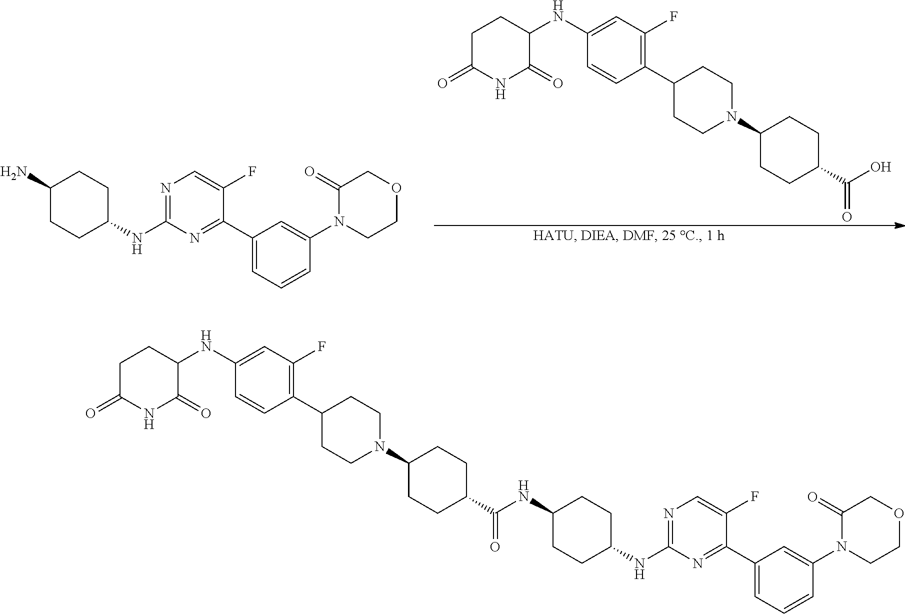 [SMILES: N[C@H]1CC[C@H](Nc2ncc(F)c(-c3cccc(N4CCOCC4=O)c3)n2)CC1 ; O=C1CCC(Nc2ccc(C3CCN([C@H]4CC[C@H](C(=O)O)CC4)CC3)c(F)c2)C(=O)N1 ; O=C1CCC(Nc2ccc(C3CCN([C@H]4CC[C@H](C(=O)N[C@H]5CC[C@H](Nc6ncc(F)c(-c7cccc(N8CCOCC8=O)c7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(3-(3-oxomorpholino)phenyl)pyrimidin-2-yl)amino)cyclohexyl)cyclohexane-1-carboxamide (Compound 568). To a solution of trans-4-(4-(4-((2,6-dioxopiperidin-3-yl)amino)-2-fluorophenyl)piperidin-1-yl)cyclohexane-1-carboxylic acid (300 mg, 695 μmol, 1.00 eq) in DMF (3 mL) were added DIEA (450 mg, 3.48 mmol, 605 μL, 5.00 eq) and HATU (79.3 mg, 209 μmol, 0.300 eq) at 25 °C. The mixture was stirred at 25 °C for 0.5 h. Then trans-4-(3-(2-((4-aminocyclohexyl)amino)-5-fluoropyrimidin-4-yl)phenyl)morpholin-3-one (174 mg, 348 mol, 0.500 eq, TFA) was added to the reaction mixture at 25 °C. The mixture was stirred at 25 °C for 0.5 h. LCMS showed trans-4-(3-(2-((4-aminocyclohexyl)amino)-5-fluoropyrimidin-4-yl)phenyl)morpholin-3-one was consumed completely and the desired mass was detected. The reaction mixture was concentrated under reduced pressure to get a residue. The residue was purified by prep-HPLC (column: Waters Xbridge Prep OBD C18 150*40 mm*10 um; mobile phase: [H₂O (10 mM NH₄HCO₃)-ACN]; gradient: 35%-65% B over 8.0 min) to give the title compound (Compound 568) (67.2 mg, 82.4 μmol, 11.9 yield, 98.0% purity) as a white solid. LCMS (ES⁺): m/z 799.4 [M+H]⁺

### Example 531. Synthesis of trans-4-(4-(4-((2,6-dioxopiperidin-3-yl)amino)-2-fluorophenyl)piperazin-1-yl)-N-(trans-4-((5-fluoro-4-(3-(3-oxomorpholino)phenyl)pyrimidin-2-yl)amino)cyclohexyl)cyclohexane-1-carboxamide (Compound 536)

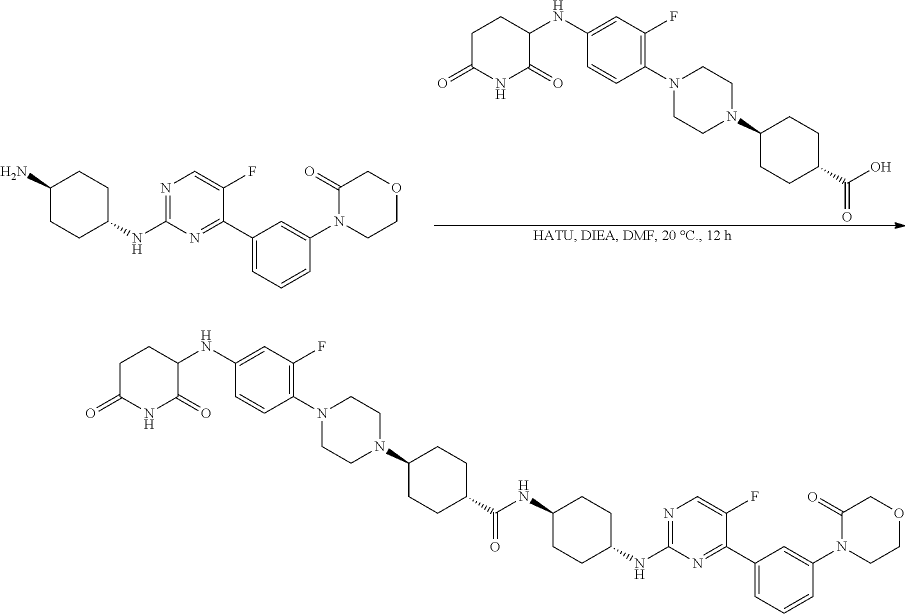 [SMILES: N[C@H]1CC[C@H](Nc2ncc(F)c(-c3cccc(N4CCOCC4=O)c3)n2)CC1 ; O=C1CCC(Nc2ccc(N3CCN([C@H]4CC[C@H](C(=O)O)CC4)CC3)c(F)c2)C(=O)N1 ; O=C1CCC(Nc2ccc(N3CCN([C@H]4CC[C@H](C(=O)N[C@H]5CC[C@H](Nc6ncc(F)c(-c7cccc(N8CCOCC8=O)c7)n6)CC5)CC4)CC3)c(F)c2)C(=O)N1]

Synthesis of trans-4-(4-(4-((2,6-dioxopiperidin-3-yl)amino)-2-fluorophenyl)piperazin-1-yl)-N-(trans-4-((5-fluoro-4-(3-(3-oxomorpholino)phenyl)pyrimidin-2-yl)amino)cyclohexyl)cyclohexane-1-carboxamide (Compound 536) To a solution of trans-4-(4-(4-((2,6-dioxopiperidin-3-yl)amino)-2-fluorophenyl)piperazin-1-yl)cyclohexane-1-carboxylic acid (216 mg, 396 μmol, 7.91e-1 eq, TFA) in DMF (5 mL) were added HATU (381 mg, 1.00 mmol, 2.00 eq), DIEA (323 mg, 2.50 mmol, 436 μL, 5.00 eq) and trans-4-(3-(2-(((1r,4r)-4-aminocyclohexyl)amino)-5-fluoropyrimidin-4-yl)phenyl)morpholin-3-one (250 mg, 501 μmol, 1.00 eq, TFA) at 20 °C. The mixture was stirred at 20 °C for 12 h. LCMS showed that trans-4-(3-(2-(((1r,4r)-4-aminocyclohexyl)amino)-5-fluoropyrimidin-4-yl)phenyl)morpholin-3-one was consumed completely and one main peak with desired MS was detected. The reaction mixture was concentrated under reduced pressure to remove solvent. The residue was purified by prep-HPLC (column: Phenomenex luna C18 100*40 mm*3 um; mobile phase: [H₂O (0.2% FA)-ACN]; gradient: 5%-40% B over 8.0 min) to give title compound (Compound 536) (218 mg, 247 μmol, 49.4% yield, 93.4% purity, 0.48 FA) as a pale yellow solid. LCMS (ES⁺): m/z 800.4 [M+H]⁺

### Example 532. Synthesis of trans-2-((1r,4R)-4-((4-(4-((2,6-dioxopiperidin-3-yl)amino)-2-fluorophenyl)piperazin-1-yl)methyl)cyclohexyl)-N-((1r,4R)-4-((5-fluoro-4-(3-(3-oxomorpholino)phenyl)pyrimidin-2-yl)amino)cyclohexyl)acetamide (Compound 562)

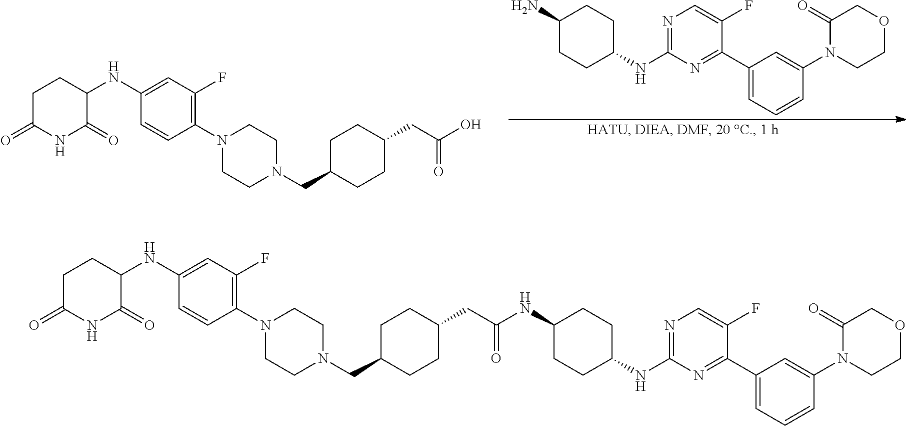 [SMILES: O=C(O)C[C@H]1CC[C@H](CN2CCN(c3ccc(NC4CCC(=O)NC4=O)cc3F)CC2)CC1 ; N[C@H]1CC[C@H](Nc2ncc(F)c(-c3cccc(N4CCOCC4=O)c3)n2)CC1 ; O=C1CCC(Nc2ccc(N3CCN(C[C@H]4CC[C@H](CC(=O)N[C@H]5CC[C@H](Nc6ncc(F)c(-c7cccc(N8CCOCC8=O)c7)n6)CC5)CC4)CC3)c(F)c2)C(=O)N1]

Synthesis of trans-2-((1r,4R)-4-((4-(4-((2,6-dioxopiperidin-3-yl)amino)-2-fluorophenyl)piperazin-1-yl)methyl)cyclohexyl)-N-((1r,4R)-4-((5-fluoro-4-(3-(3-oxomorpholino)phenyl)pyrimidin-2-yl)amino)cyclohexyl)acetamide (Compound 562). To a solution of trans-2-(4-((4-(4-((2,6-dioxopiperidin-3-yl)amino)-2-fluorophenyl)piperazin-1-yl)methyl)cyclohexyl)acetic acid (445 mg, 966 μmol, 1.00 eq) in DMF (8 mL) were added HATU (129 mg, 338 μmol, 0.350 eq) and DIEA (625 mg, 4.83 mmol, 843 μL, 5.00 eq) at 20 °C, then to the mixture was added trans-4-(3-(2-((4-aminocyclohexyl)amino)-5-fluoropyrimidin-4-yl)phenyl)morpholin-3-one (434 mg, 870 μmol, 0.900 eq, TFA) at 20 °C, the mixture was stirred at 20 °C for 1 h. LCMS showed trans-2-(4-((4-(4-((2,6-dioxopiperidin-3-yl)amino)-2-fluorophenyl)piperazin-1-yl)methyl)cyclohexyl)acetic acid was consumed completely and the desired mass was detected. The mixture was concentrated under reduced pressure to give a residue. The residue was purified by prep-HPLC (column: Waters Xbridge BEH C18 100*25 mm*10 um; mobile phase: [H₂O (10 mM NH₄HCO₃)-ACN]; gradient: 30%-60% B over 8.0 min) to give the title compound (Compound 562) (153 mg, 179 μmol, 18.5% yield, 96.8% purity) as a yellow solid. LCMS (ES⁺): m/z 828.4 [M+H]⁺

### Example 533. Synthesis of trans-1′-(4-((2,6-dioxopiperidin-3-yl)amino)-2-fluorophenyl)-N-(4-((5-fluoro-4-(3-(3-oxomorpholino)phenyl)pyrimidin-2-yl)amino)cyclohexyl)-[1,4′-bipiperidine]-4-carboxamide (Compound 585)

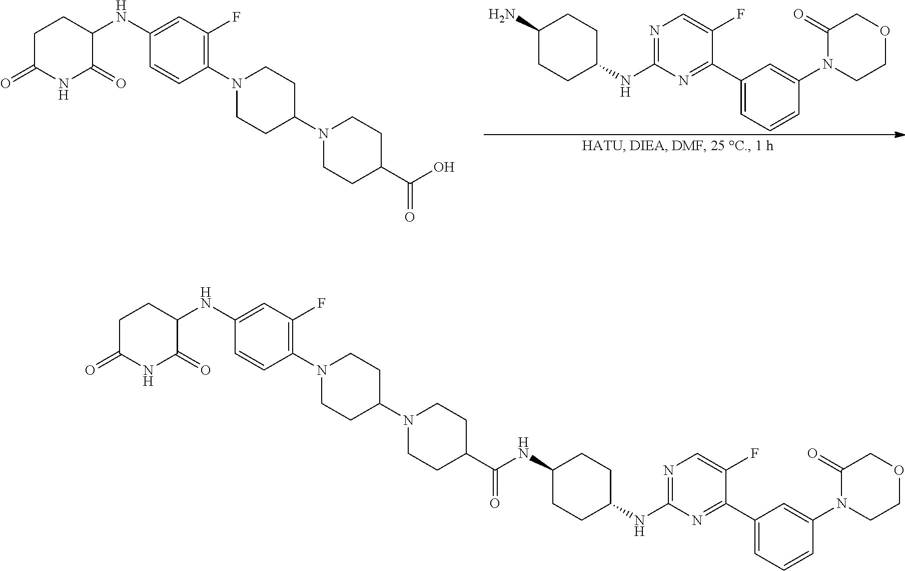 [SMILES: O=C1CCC(Nc2ccc(N3CCC(N4CCC(C(=O)O)CC4)CC3)c(F)c2)C(=O)N1 ; N[C@H]1CC[C@H](Nc2ncc(F)c(-c3cccc(N4CCOCC4=O)c3)n2)CC1 ; O=C1CCC(Nc2ccc(N3CCC(N4CCC(C(=O)N[C@H]5CC[C@H](Nc6ncc(F)c(-c7cccc(N8CCOCC8=O)c7)n6)CC5)CC4)CC3)c(F)c2)C(=O)N1]

Synthesis of trans-1′-(4-((2,6-dioxopiperidin-3-yl)amino)-2-fluorophenyl)-N-(4-((5-fluoro-4-(3-(3-oxomorpholino)phenyl)pyrimidin-2-yl)amino)cyclohexyl)-[1,4′-bipiperidine]-4-carboxamide (Compound 585). To a solution of 1′-(4-((2,6-dioxopiperidin-3-yl)amino)-2-fluorophenyl)-[1,4′-bipiperidine]-4-carboxylic acid (820 mg, 1.90 mmol, 1.00 eq) in DMF (20 mL) were added HATU (721 mg, 1.90 mmol, 1.00 eq), DIEA (735 mg, 5.69 mmol, 991 μL, 3.00 eq) and trans-4-(3-(2-((4-aminocyclohexyl)amino)-5-fluoropyrimidin-4-yl)phenyl)morpholin-3-one (938 mg, 1.88 mmol, 0.990 eq, TFA) at 25 °C. The mixture was stirred at 25 °C for 1 h. LC-MS showed the desired mass was detected. The reaction mixture was concentrated under reduced pressure to give a residue. The residue was purified by prep-HPLC (column: Waters Xbridge BEH C18 250*50 mm*10 um; mobile phase: [H₂O (10 mM NH₄HCO₃)-ACN]; gradient: 25%-55% B over 10.0 min) to give the title compound (Compound 585) (181 mg, 209 μmol, 11.0% yield, 92.2% purity) as a white solid. LCMS (ES⁺): m/z 800.5 [M+H]⁺

### Example 534. Synthesis of 1′-(4-((2,6-dioxopiperidin-3-yl)amino)-2-fluorophenyl)-3′-fluoro-N-((1r,4r)-4-((5-fluoro-4-(3-(3-oxomorpholino)phenyl)pyrimidin-2-yl)amino)cyclohexyl)-[1,4′-bipiperidine]-4-carboxamide (Compound 526)

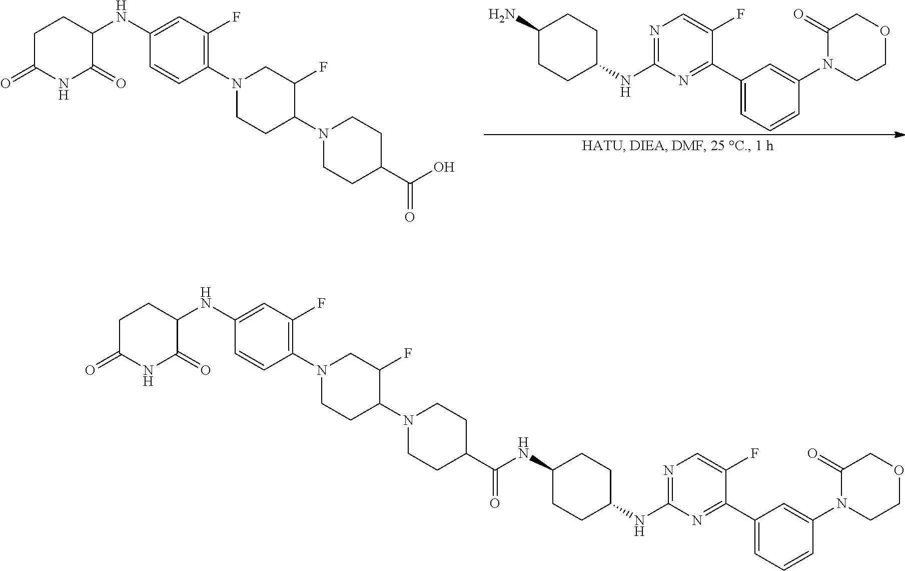 [SMILES: O=C1CCC(Nc2ccc(N3CCC(N4CCC(C(=O)O)CC4)C(F)C3)c(F)c2)C(=O)N1 ; N[C@H]1CC[C@H](Nc2ncc(F)c(-c3cccc(N4CCOCC4=O)c3)n2)CC1 ; O=C1CCC(Nc2ccc(N3CCC(N4CCC(C(=O)N[C@H]5CC[C@H](Nc6ncc(F)c(-c7cccc(N8CCOCC8=O)c7)n6)CC5)CC4)C(F)C3)c(F)c2)C(=O)N1]

Synthesis of 1′-(4-((2,6-dioxopiperidin-3-yl)amino)-2-fluorophenyl)-3′-fluoro-N-((1r,4r)-4-((5-fluoro-4-(3-(3-oxomorpholino)phenyl)pyrimidin-2-yl)amino)cyclohexyl)-[1,4′-bipiperidine]-4-carboxamide (Compound 526). To a solution of 1′-(4-((2,6-dioxopiperidin-3-yl)amino)-2-fluorophenyl)-3′-fluoro-[1,4′-bipiperidine]-4-carboxylic acid (260 mg, 577 μmol, 1.00 eq) in DMF (5 mL) were added HATU (76.8 mg, 202 μmol, 0.350 eq) and DIEA (373 mg, 2.88 mmol, 503 μL, 5.00 eq) at 25 °C, then to the reaction was added trans-4-(3-(2-((4-aminocyclohexyl)amino)-5-fluoropyrimidin-4-yl)phenyl)morpholin-3-one (202 mg, 404 μmol, 0.700 eq, TFA) at 25 °C, the mixture was stirred at 25 °C for 1 h. LCMS showed 1′-(4-((2,6-dioxopiperidin-3-yl)amino)-2-fluorophenyl)-3′-fluoro-[1,4′-bipiperidine]-4-carboxylic acid was consumed completely and the desired mass was detected. The mixture was concentrated under reduced pressure to give a residue. The residue was purified by acidic prep-HPLC (column: Phenomenex Luna C18 75*30 mm*3 um; mobile phase: [H₂O (0.2% FA)-ACN]; gradient: 10%-50% B over 8.0 min) to give the title compound (Compound 526) (80.2 mg, 96.1 μmol, 16.6% yield, 98.0% purity) as a yellow solid. LCMS (ES⁺): m/z 818.1 [M+H]⁺

### Example 535. Synthesis of trans-1′-(4-((2,6-dioxopiperidin-3-yl)amino)-2-fluorophenyl)-3′,3′-difluoro-N-(4-((5-fluoro-4-(3-(3-oxomorpholino)phenyl)pyrimidin-2-yl)amino)cyclohexyl)-[1,4′-bipiperidine]-4-carboxamide (Compound 501)

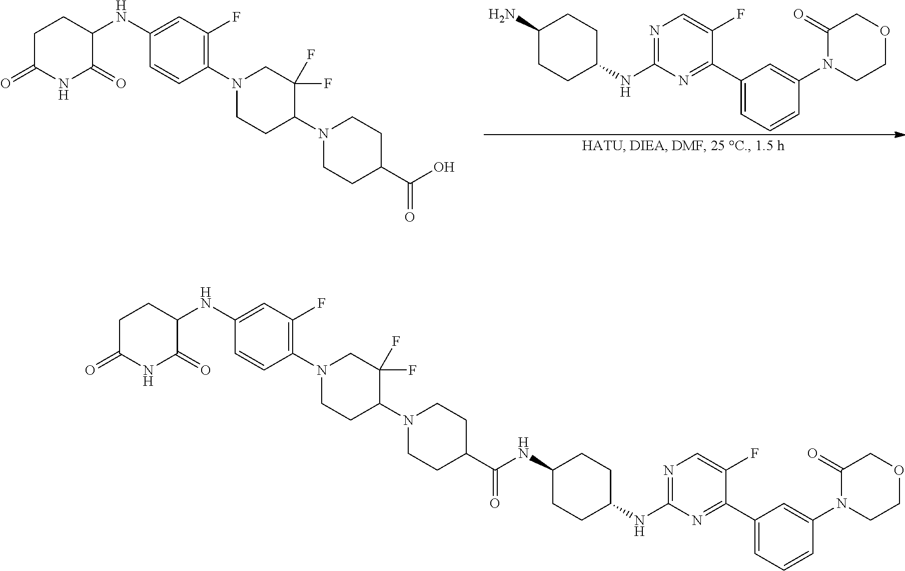 [SMILES: O=C1CCC(Nc2ccc(N3CCC(N4CCC(C(=O)O)CC4)C(F)(F)C3)c(F)c2)C(=O)N1 ; N[C@H]1CC[C@H](Nc2ncc(F)c(-c3cccc(N4CCOCC4=O)c3)n2)CC1 ; O=C1CCC(Nc2ccc(N3CCC(N4CCC(C(=O)N[C@H]5CC[C@H](Nc6ncc(F)c(-c7cccc(N8CCOCC8=O)c7)n6)CC5)CC4)C(F)(F)C3)c(F)c2)C(=O)N1]

Synthesis of trans-1′-(4-((2,6-dioxopiperidin-3-yl)amino)-2-fluorophenyl)-3′,3′-difluoro-N-(4-((5-fluoro-4-(3-(3-oxomorpholino)phenyl)pyrimidin-2-yl)amino)cyclohexyl)-[1,4′-bipiperidine]-4-carboxamide (Compound 501). To a solution of 1′-(4-((2,6-dioxopiperidin-3-yl)amino)-2-fluorophenyl)-3′,3′-difluoro-[1,4′-bipiperidine]-4-carboxylic acid (200 mg, 427 μmol, 1.00 eq) in DMF (6 mL) were added DIEA (277 mg, 2.13 mmol, 372 μL, 5.00 eq) and HATU (61.7 mg, 162 μmol, 0.380 eq) at 25 °C. The mixture was stirred at 25 °C for 0.5 h. Then trans-4-(3-(2-((4-aminocyclohexyl)amino)-5-fluoropyrimidin-4-yl)phenyl)morpholin-3-one (65.8 mg, 132 μmol, 0.309 eq, TFA) was added to the reaction mixture at 25 °C. The mixture was stirred at 25 °C for 1 h. LCMS showed 1′-(4-((2,6-dioxopiperidin-3-yl)amino)-2-fluorophenyl)-3′,3′-difluoro-[1,4′-bipiperidine]-4-carboxylic acid was consumed completely and the desired mass was detected. The mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 1%-40% B over 8.0 min) to give the title compound (Compound 501) (32.5 mg, 36.8 μmol, 8.61% yield, 94.6% purity) as a pale yellow solid. LCMS (ES⁺): m/z 836.4 [M+H]⁺

### Example 536. Synthesis of trans-N-(trans-4-(4-(4-((2,6-dioxopiperidin-3-yl)amino)-2-fluorophenyl)piperidin-1-yl)cyclohexyl)-4-((5-fluoro-4-(3-(3-oxomorpholino)phenyl)pyrimidin-2-yl)amino)cyclohexane-1-carboxamide (Compound 547)

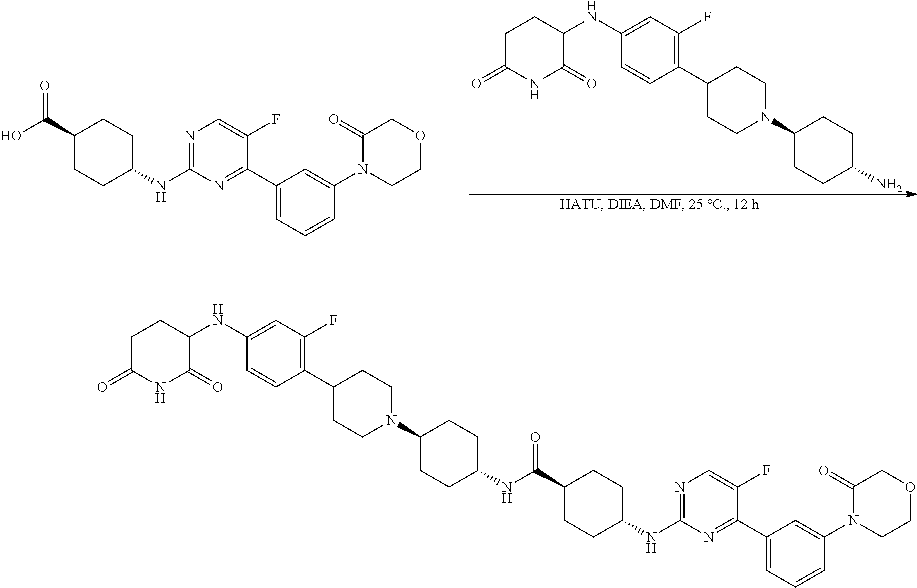 [SMILES: O=C1COCCN1c1cccc(-c2nc(N[C@H]3CC[C@H](C(=O)O)CC3)ncc2F)c1 ; N[C@H]1CC[C@H](N2CCC(c3ccc(NC4CCC(=O)NC4=O)cc3F)CC2)CC1 ; O=C1CCC(Nc2ccc(C3CCN([C@H]4CC[C@H](NC(=O)[C@H]5CC[C@H](Nc6ncc(F)c(-c7cccc(N8CCOCC8=O)c7)n6)CC5)CC4)CC3)c(F)c2)C(=O)N1]

Synthesis of trans-N-(trans-4-(4-(4-((2,6-dioxopiperidin-3-yl)amino)-2-fluorophenyl)piperidin-1-yl)cyclohexyl)-4-((5-fluoro-4-(3-(3-oxomorpholino)phenyl)pyrimidin-2-yl)amino)cyclohexane-1-carboxamide (Compound 547). To a solution of trans-4-[[5-fluoro-4-[3-(3-oxomorpholin-4-yl)phenyl]pyrimidin-2-yl]amino]cyclohexanecarboxylic acid (300 mg, 567 μmol, 1.00 eq, TFA) in DMF (1 mL) was added DIEA (73.3 mg, 567 μmol, 98.8 μL, 1.00 eq) until pH~7. To a solution of trans-3-((4-(1-(4-aminocyclohexyl)piperidin-4-yl)-3-fluorophenyl)amino)piperidine-2,6-dione (297 mg, 575 μmol, 1.01 eq, TFA) in DMF (2 mL) was added DIEA (73.3 mg, 567.7 μmol, 98.8 μL, 1.00 eq) until pH~7. Then HATU (215 mg, 567 μmol, 1.00 eq) was added to the mixture of the two solutions. The mixture was stirred at 25 °C for 12 h. LC-MS showed the desired mass was detected. The reaction mixture was filtered. The residue was purified by prep-HPLC (column: Waters Xbridge Prep OBD C18 150*40 mm*10 um; mobile phase: [H₂O (10 mM NH₄HCO₃)-ACN]; gradient: 25%-55% B over 8.0 min) to give the title compound (Compound 547) (219 mg, 266 μmol, 36.8% yield, 97.1% purity) as a white solid. LCMS (ES⁺): m/z 799.5 [M+H]⁺.

### Example 537. Synthesis of trans-N-(trans-4-(4-(4-((2,6-dioxopiperidin-3-yl)amino)-2-fluorophenyl)piperazin-1-yl)cyclohexyl)-4-((5-fluoro-4-(3-(3-oxomorpholino)phenyl)pyrimidin-2-yl)amino)cyclohexane-1-carboxamide (Compound 508)

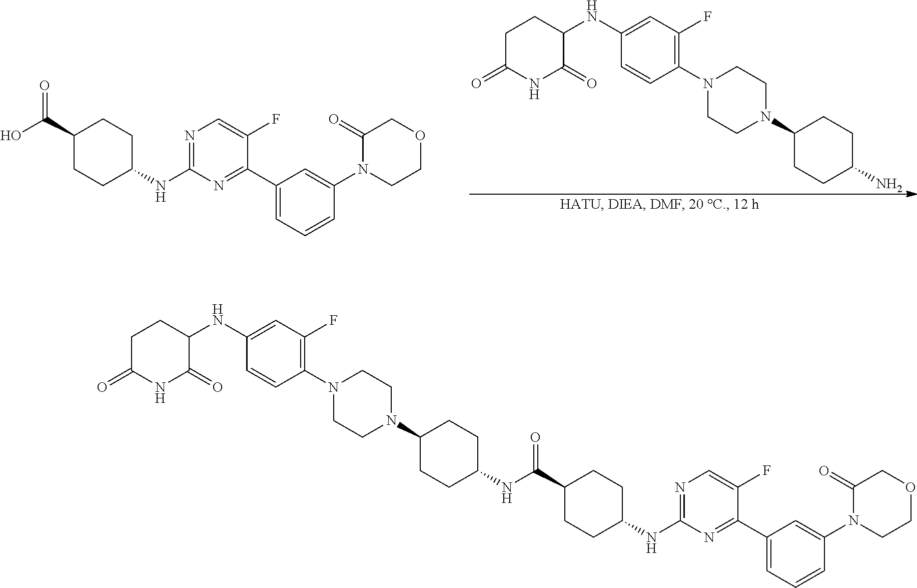 [SMILES: O=C1COCCN1c1cccc(-c2nc(N[C@H]3CC[C@H](C(=O)O)CC3)ncc2F)c1 ; N[C@H]1CC[C@H](N2CCN(c3ccc(NC4CCC(=O)NC4=O)cc3F)CC2)CC1 ; O=C1CCC(Nc2ccc(N3CCN([C@H]4CC[C@H](NC(=O)[C@H]5CC[C@H](Nc6ncc(F)c(-c7cccc(N8CCOCC8=O)c7)n6)CC5)CC4)CC3)c(F)c2)C(=O)N1]

Synthesis of trans-N-(trans-4-(4-(4-((2,6-dioxopiperidin-3-yl)amino)-2-fluorophenyl)piperazin-1-yl)cyclohexyl)-4-((5-fluoro-4-(3-(3-oxomorpholino)phenyl)pyrimidin-2-yl)amino)cyclohexane-1-carboxamide (Compound 508). To a solution of trans-(1r,4r)-4-((5-fluoro-4-(3-(3-oxomorpholino)phenyl)pyrimidin-2-yl)amino)cyclohexane-1-carboxylic acid (160 mg, 386 μmol, 1.00 eq) in DMF (5 mL) was added DIEA (249 mg, 1.93 mmol, 336 μL, 5.00 eq), HATU (294 mg, 772 μmol, 2.00 eq) and trans-3-((4-(4-(4-aminocyclohexyl)piperazin-1-yl)-3-fluorophenyl)amino)piperidine-2,6-dione (250 mg, 483 μmol, 1.25 eq, TFA) at 20 °C. The mixture was stirred at 20 °C for 12 h. LCMS showed that trans-(1r,4r)-4-((5-fluoro-4-(3-(3-oxomorpholino)phenyl)pyrimidin-2-yl)amino)cyclohexane-1-carboxylic acid was consumed completely and one main peak with desired MS was detected. The reaction mixture was concentrated under reduced pressure to remove solvent. The residue was purified by prep-HPLC (column: Phenomenex luna C18 100*40 mm*5 um; mobile phase: [H₂O (0.2% FA)-ACN]; gradient: 5%-40% B over 8.0 min) to give title compound (Compound 508) (69.8 mg, 86.6 μmol, 22.4% yield, 100% purity, 0.14 FA) as a pale yellow solid. LCMS (ES⁺): m/z 800.4 [M+H]⁺

### Example 538. Synthesis of trans-3-((3-fluoro-4-(4-((1-4-((5-fluoro-4-(3-(3-oxomorpholino)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 588)

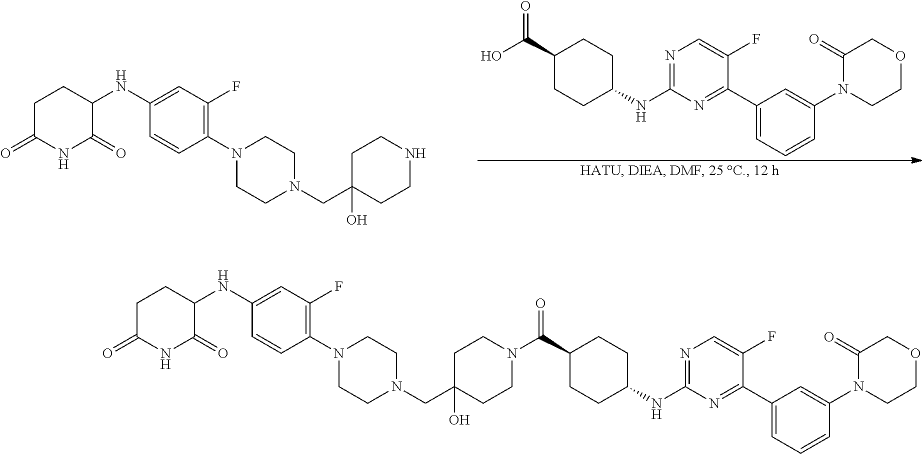 [SMILES: O=C1CCC(Nc2ccc(N3CCN(CC4(O)CCNCC4)CC3)c(F)c2)C(=O)N1 ; O=C1COCCN1c1cccc(-c2nc(N[C@H]3CC[C@H](C(=O)O)CC3)ncc2F)c1 ; O=C1CCC(Nc2ccc(N3CCN(CC4(O)CCN(C(=O)[C@H]5CC[C@H](Nc6ncc(F)c(-c7cccc(N8CCOCC8=O)c7)n6)CC5)CC4)CC3)c(F)c2)C(=O)N1]

Synthesis of trans-3-((3-fluoro-4-(4-((1-4-((5-fluoro-4-(3-(3-oxomorpholino)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 588). To a solution of trans-4-[[5-fluoro-4-[3-(3-oxomorpholin-4-yl)phenyl]pyrimidin-2-yl]amino]cyclohexanecarboxylic acid (500 mg, 1.21 mmol, 1.00 eq) in DMF (1 mL) was added DIEA (155 mg, 1.21 mmol, 210 μL, 1.00 eq) until pH~7. To a solution of 3-((3-fluoro-4-(4-((4-hydroxypiperidin-4-yl) methyl) piperazin-1-yl) phenyl) amino) piperidine-2, 6-dione (759 mg, 1.81 mmol, 1.50 eq) in DMF (2 mL) was added DIEA (155 mg, 1.21 mmol, 210 μL, 1.00 eq) until pH~7. Then HATU (458 mg, 1.21 mmol, 1.00 eq) was added to the mixture of the two solutions. The mixture was stirred at 25 °C for 12 h. LC-MS showed the desired mass was detected. The reaction mixture was filtered. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to give the title compound (Compound 588) (331 mg, 385 μmol, 31.9% yield, 95.0% purity) as a pale yellow solid. LCMS (ES⁺): m/z 816.4 [M+H]⁺.

### Example 539. Synthesis of trans-3-((3-fluoro-4-(1-((1-(4-((5-fluoro-4-(3-(3-oxomorpholino)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 578)

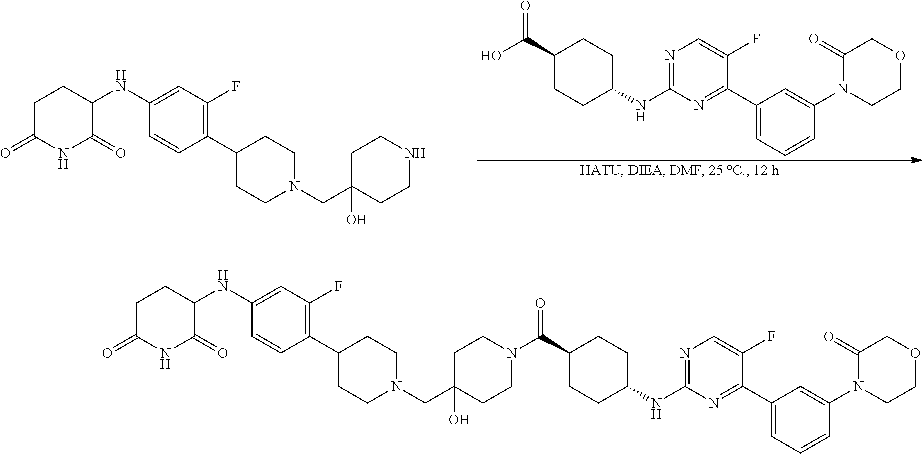 [SMILES: O=C1CCC(Nc2ccc(C3CCN(CC4(O)CCNCC4)CC3)c(F)c2)C(=O)N1 ; O=C1COCCN1c1cccc(-c2nc(N[C@H]3CC[C@H](C(=O)O)CC3)ncc2F)c1 ; O=C1CCC(Nc2ccc(C3CCN(CC4(O)CCN(C(=O)[C@H]5CC[C@H](Nc6ncc(F)c(-c7cccc(N8CCOCC8=O)c7)n6)CC5)CC4)CC3)c(F)c2)C(=O)N1]

Synthesis of trans-3-((3-fluoro-4-(1-((1-(4-((5-fluoro-4-(3-(3-oxomorpholino)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 578). To a solution of trans-4-[[5-fluoro-4-[3-(3-oxomorpholin-4-yl)phenyl]pyrimidin-2-yl]amino]cyclohexanecarboxylic acid (340 mg, 820 μmol, 1.00 eq) in DMF (1 mL) was added DIEA (106 mg, 820 μmol, 142 μL, 1.00 eq) until pH~7. To a solution of 3-((3-fluoro-4-(1-((4-hydroxypiperidin-4-yl)methyl)piperidin-4-yl)phenyl)amino)piperidine-2,6-dione (686 mg, 1.64 mmol, 2.00 eq) in DMF (2 mL) was added DIEA (106 mg, 820 μmol, 142 μL, 1.00 eq) until pH~7. Then HATU (623 mg, 1.64 mmol, 2.00 eq) was added to the mixture of the two solutions. The mixture was stirred at 25 °C for 12 h. LC-MS showed the desired mass was detected. The reaction mixture was filtered. The residue was purified by prep-HPLC (column: Waters Xbridge Prep OBD C18 150*40 mm*10 um; mobile phase: [H₂O (10 mM NH₄HCO₃)-ACN]; gradient: 30%-65% B over 8.0 min) to give the title compound (Compound 578) (168 mg, 202 μmol, 24.6% yield, 98.1% purity) as an off white solid. LCMS (ES⁺): m/z 815.4 [M+H]⁺.

### Example 540. Synthesis of trans-(1r,4R)—N-(((1r,4R)-4-((4-(4-((2,6-dioxopiperidin-3-yl)amino)-2-fluorophenyl)piperazin-1-yl)methyl)cyclohexyl)methyl)-4-((5-fluoro-4-(3-(3-oxomorpholino)phenyl)pyrimidin-2-yl)amino)cyclohexane-1-carboxamide (Compound 586)

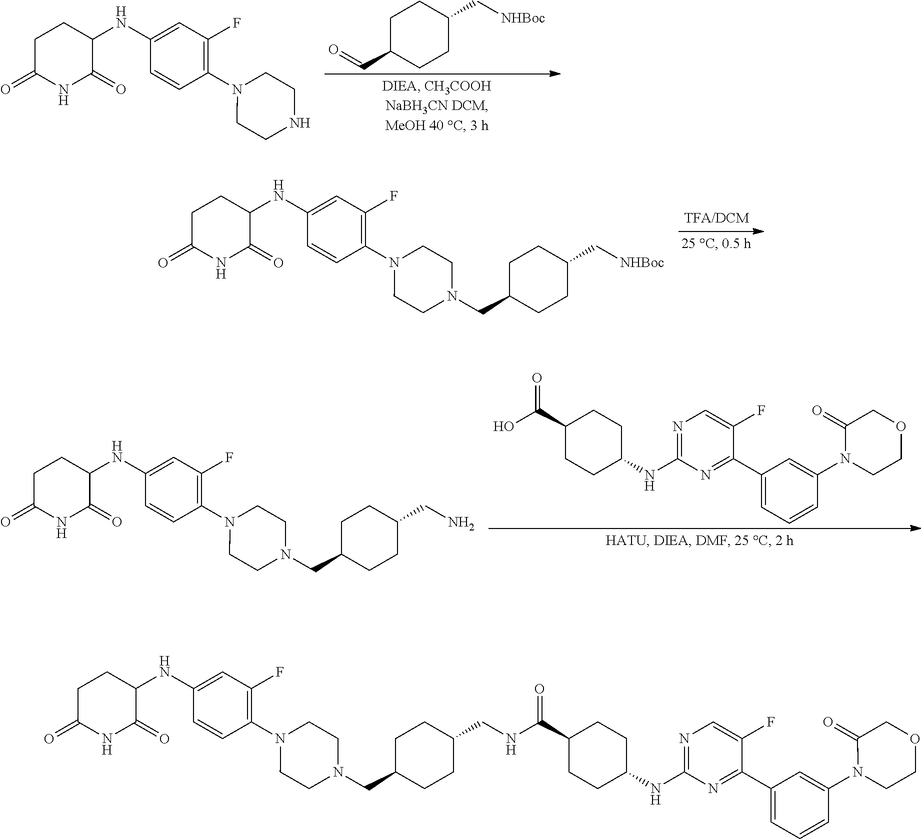 [SMILES: O=C1CCC(Nc2ccc(N3CCNCC3)c(F)c2)C(=O)N1 ; CC(C)(C)OC(=O)NC[C@H]1CC[C@H](C=O)CC1 ; CC(C)(C)OC(=O)NC[C@H]1CC[C@H](CN2CCN(c3ccc(NC4CCC(=O)NC4=O)cc3F)CC2)CC1 ; NC[C@H]1CC[C@H](CN2CCN(c3ccc(NC4CCC(=O)NC4=O)cc3F)CC2)CC1 ; O=C1COCCN1c1cccc(-c2nc(N[C@H]3CC[C@H](C(=O)O)CC3)ncc2F)c1 ; O=C1CCC(Nc2ccc(N3CCN(C[C@H]4CC[C@H](CNC(=O)[C@H]5CC[C@H](Nc6ncc(F)c(-c7cccc(N8CCOCC8=O)c7)n6)CC5)CC4)CC3)c(F)c2)C(=O)N1]

Synthesis of trans-tert-butyl ((4-((4-(4-((2,6-dioxopiperidin-3-yl)amino)-2-fluorophenyl)piperazin-1-yl)methyl)cyclohexyl)methyl)carbamate. To a solution of 3-((3-fluoro-4-(piperazin-1-yl)phenyl)amino)piperidine-2,6-dione (2.10 g, 5.00 mmol, 1.00 eq, TFA) in DCM (40 mL) and MeOH (8 mL) was added DIEA to adjust pH to 8 at 25 °C. Then to the reaction was added CH3COOH (900 mg, 15.0 mmol, 858 μL, 3.00 eq) and trans-tert-butyl ((4-formylcyclohexyl)methyl)carbamate (1.81 g, 7.49 mmol, 1.50 eq) at 25 °C, the pH was 5 now. The mixture was stirred at 40 °C for 1 h. To the reaction mixture was added NaBH3CN (942 mg, 15.0 mmol, 3.00 eq) at 25 °C and the resulting mixture was stirred at 40 °C for 2 h. LCMS showed 3-((3-fluoro-4-(piperazin-1-yl)phenyl)amino)piperidine-2,6-dione was consumed and the desired mass was detected. The mixture was concentrated under reduced pressure to give the residue. To the residue was added H₂O (50 mL) and the mixture was extracted with EtOAc (50 mL*3). The combined organic layers were washed with brine (50 mL*3), dried over Na₂SO₄, filtered and the filtrate was concentrated under reduced pressure to give a residue. The residue was purified by MPLC (SiO2, PE/EtOAc=1/0 to 0/1) to give the title compound (1.00 g, 1.88 mmol, 37.7% yield) as a yellow oil. LCMS (ES+): m/z 532.4 [M+H]+.

Synthesis of trans-3-((4-(4-((4-(aminomethyl)cyclohexyl)methyl)piperazin-1-yl)-3-fluorophenyl)amino)piperidine-2,6-dione. A solution of trans-tert-butyl ((4-((4-(4-((2,6-dioxopiperidin-3-yl)amino)-2-fluorophenyl)piperazin-1-yl)methyl)cyclohexyl)methyl)carbamate (600 mg, 1.13 mmol, 1.00 eq) in DCM (6 mL) and TFA (2 mL) was stirred at 25 °C for 0.5 h. LCMS showed trans-tert-butyl ((4-((4-(4-((2,6-dioxopiperidin-3-yl)amino)-2-fluorophenyl)piperazin-1-yl)methyl)cyclohexyl)methyl)carbamate was consumed completely and the desired mass was detected. The mixture was concentrated under reduced pressure to give the title compound (616 mg, crude, TFA) as a yellow oil. LCMS (ES+): m/z 432.2 [M+H]+.

Synthesis of trans-(1r,4R)—N-(((1r,4R)-4-((4-(4-((2,6-dioxopiperidin-3-yl)amino)-2-fluorophenyl)piperazin-1-yl)methyl)cyclohexyl)methyl)-4-((5-fluoro-4-(3-(3-oxomorpholino)phenyl)pyrimidin-2-yl)amino)cyclohexane-1-carboxamide (Compound 586). To a solution of trans-4-((5-fluoro-4-(3-(3-oxomorpholino)phenyl)pyrimidin-2-yl)amino)cyclohexane-1-carboxylic acid (281 mg, 677 μmol, 0.600 eq) in DMF (5 mL) were added DIEA (730 mg, 2.65 mmol, 983 μL, 5.00 eq) and HATU (429 mg, 1.13 mmol, 1.00 eq) at 25 °C. Then to the mixture was added trans-3-((4-(4-((4-(aminomethyl)cyclohexyl)methyl)piperazin-1-yl)-3-fluorophenyl)amino)piperidine-2,6-dione (616 mg, 1.13 mmol, 1.00 eq, TFA) at 25 °C. The mixture was stirred at 25 °C for 2 h. LCMS showed trans-3-((4-(4-((4-(aminomethyl)cyclohexyl)methyl)piperazin-1-yl)-3-fluorophenyl)amino)piperidine-2,6-dione was consumed and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH4HCO3)-ACN]; gradient: 35%-65% B over 8.0 min) to give the title compound (Compound 586) (295 mg, 332 μmol, 29.4% yield, 93.3% purity) as a white solid. LCMS (ES+): m/z 828.4 [M+H]+

### Example 541. Synthesis of trans-(1r,4R)—N-(((1r,4R)-4-((4-(4-((2,6-dioxopiperidin-3-yl)amino)-2-fluorophenyl)piperidin-1-yl)methyl)cyclohexyl)methyl)-4-((5-fluoro-4-(3-(3-oxomorpholino)phenyl)pyrimidin-2-yl)amino)cyclohexane-1-carboxamide (Compound 589)

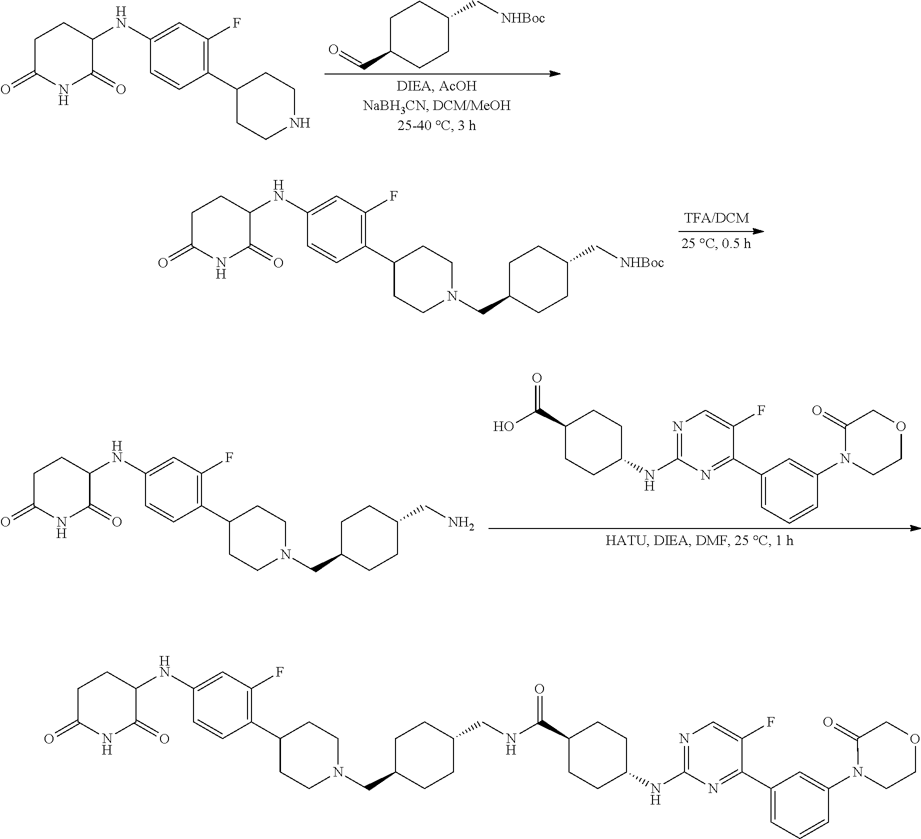 [SMILES: O=C1CCC(Nc2ccc(C3CCNCC3)c(F)c2)C(=O)N1 ; CC(C)(C)OC(=O)NC[C@H]1CC[C@H](C=O)CC1 ; CC(C)(C)OC(=O)NC[C@H]1CC[C@H](CN2CCC(c3ccc(NC4CCC(=O)NC4=O)cc3F)CC2)CC1 ; NC[C@H]1CC[C@H](CN2CCC(c3ccc(NC4CCC(=O)NC4=O)cc3F)CC2)CC1 ; O=C1COCCN1c1cccc(-c2nc(N[C@H]3CC[C@H](C(=O)O)CC3)ncc2F)c1 ; O=C1CCC(Nc2ccc(C3CCN(C[C@H]4CC[C@H](CNC(=O)[C@H]5CC[C@H](Nc6ncc(F)c(-c7cccc(N8CCOCC8=O)c7)n6)CC5)CC4)CC3)c(F)c2)C(=O)N1]

Synthesis of trans-tert-butyl ((4-((4-(4-((2,6-dioxopiperidin-3-yl)amino)-2-fluorophenyl)piperidin-1-yl)methyl)cyclohexyl)methyl)carbamate. To a solution of 3-((3-fluoro-4-(piperidin-4-yl)phenyl)amino)piperidine-2,6-dione (5.00 g, 11.9 mmol, 1.00 eq, TFA) in DCM (100 mL) and MeOH (20 mL) was added DIEA at 25 °C until the pH had reached 8. The mixture was stirred at 25 °C for 0.5 h. To the mixture were added CH3COOH (3.58 g, 59.5 mmol, 3.42 mL, 5.00 eq) and trans-tert-butyl ((4-formylcyclohexyl)methyl)carbamate (4.32 g, 17.9 mmol, 1.50 eq) at 25 °C, pH was 5 now. The mixture was stirred at 40 °C for 0.5 h. Then NaBH3CN (2.25 g, 35.8 mmol, 3.00 eq) was added to the mixture at 25 °C. The mixture was stirred at 40 °C for 2 h. LCMS showed 3-((3-fluoro-4-(piperidin-4-yl)phenyl)amino)piperidine-2,6-dione was consumed completely and the desired mass was detected. The mixture was concentrated under reduced pressure to give a residue. To the residue was added H₂O (100 mL) and then extracted with DCM (100 mL*3). The combined organic layers were dried over Na₂SO₄, filtered and the filtrate was concentrated under reduced pressure to give a residue. The residue was purified by MPLC (SiO2, PE/EtOAc=1/0 to 0/1) to give the title compound (1.60 g, 3.02 mmol, 25.3% yield) as a white solid. LCMS (ES+): m/z 531.2 [M+H]+

Synthesis of trans-3-((4-(1-((4-(aminomethyl)cyclohexyl)methyl)piperidin-4-yl)-3-fluorophenyl)amino)piperidine-2,6-dione. A solution of trans-tert-butyl ((4-((4-(4-((2,6-dioxopiperidin-3-yl)amino)-2-fluorophenyl)piperidin-1-yl)methyl)cyclohexyl)methyl)carbamate (512 mg, 965 μmol, 1.00 eq) in DCM (4.5 mL) and TFA (1.5 mL) was stirred at 25 °C for 0.5 h. LCMS showed trans-tert-butyl ((4-((4-(4-((2,6-dioxopiperidin-3-yl)amino)-2-fluorophenyl)piperidin-1-yl)methyl)cyclohexyl)methyl)carbamate was consumed completely and the desired mass was detected. The mixture was concentrated under reduced pressure to give the title compound (512 mg, crude, TFA) as a yellow oil. LCMS (ES+): m/z 431.2 [M+H]+

Synthesis of trans-(1r,4R)—N-(((1r,4R)-4-((4-(4-((2,6-dioxopiperidin-3-yl)amino)-2-fluorophenyl)piperidin-1-yl)methyl)cyclohexyl)methyl)-4-((5-fluoro-4-(3-(3-oxomorpholino)phenyl)pyrimidin-2-yl)amino)cyclohexane-1-carboxamide (Compound 589). To a solution of trans-4-((5-fluoro-4-(3-(3-oxomorpholino)phenyl)pyrimidin-2-yl)amino)cyclohexane-1-carboxylic acid (389 mg, 939 μmol, 1.00 eq) in DMF (8 mL) were added HATU (535 mg, 1.41 mmol, 1.50 eq) and DIEA (608 mg, 4.70 mmol, 818 μL, 5.00 eq) at 25 °C, then to the mixture was added trans-3-((4-(1-((4-(aminomethyl)cyclohexyl)methyl)piperidin-4-yl)-3-fluorophenyl)amino)piperidine-2,6-dione (511 mg, 939 μmol, 1.00 eq, TFA) at 25 °C, the mixture was stirred at 25 °C for 1 h. LCMS showed trans-3-((4-(1-((4-(aminomethyl)cyclohexyl)methyl)piperidin-4-yl)-3-fluorophenyl)amino)piperidine-2,6-dione was consumed completely and the desired mass was detected. To the mixture was added H₂O (10 mL) and then the mixture was extracted with EtOAc (10 mL*3). The combined organic layers were dried over Na₂SO₄, filtered and the filtrate was concentrated under reduced pressure to give the residue. The residue was purified by prep-HPLC (column: Waters Xbridge BEH C18 100*30 mm*10 um; mobile phase: [H₂O (10 mM NH₄HCO₃)-ACN]; gradient: 25%-65% B over 8.0 min) to give the title compound (Compound 589) (86.7 mg, 98.2 μmol, 10.5% yield, 93.7% purity) as a yellow solid. LCMS (ES⁺): m/z 827.4 [M+H]⁺

### Example 542. Synthesis of trans-N-(1′-(4-((2,6-dioxopiperidin-3-yl)amino)-2-fluorophenyl)-[1,4′-bipiperidin]-4-yl)-4-((5-fluoro-4-(3-(3-oxomorpholino)phenyl)pyrimidin-2-yl)amino)cyclohexane-1-carboxamide (Compound 532)

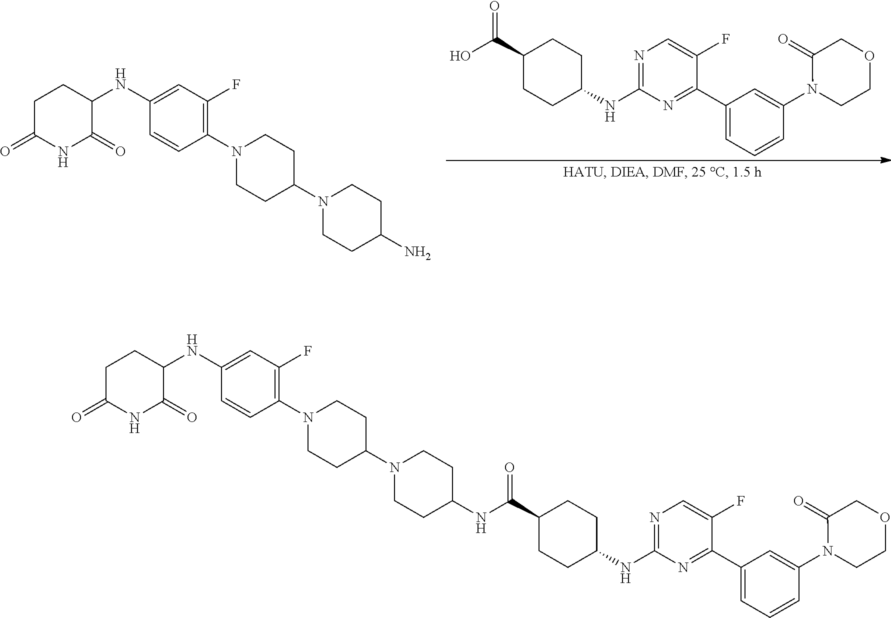 [SMILES: NC1CCN(C2CCN(c3ccc(NC4CCC(=O)NC4=O)cc3F)CC2)CC1 ; O=C1COCCN1c1cccc(-c2nc(N[C@H]3CC[C@H](C(=O)O)CC3)ncc2F)c1 ; O=C1CCC(Nc2ccc(N3CCC(N4CCC(NC(=O)[C@H]5CC[C@H](Nc6ncc(F)c(-c7cccc(N8CCOCC8=O)c7)n6)CC5)CC4)CC3)c(F)c2)C(=O)N1]

Synthesis of trans-N-(1′-(4-((2,6-dioxopiperidin-3-yl)amino)-2-fluorophenyl)-[1,4′-bipiperidin]-4-yl)-4-((5-fluoro-4-(3-(3-oxomorpholino)phenyl)pyrimidin-2-yl)amino)cyclohexane-1-carboxamide (Compound 532). To a solution of trans-4-[[5-fluoro-4-[3-(3-oxomorpholin-4-yl)phenyl]pyrimidin-2-yl]amino]cyclohexanecarboxylic acid (96.1 mg, 232 μmol, 0.400 eq) in DMF (3 mL) were added DIEA (375 mg, 2.90 mmol, 505 μL, 5.00 eq) and HATU (77.2 mg, 203 μmol, 0.350 eq) at 25 °C. The mixture was stirred at 25 °C for 0.5 h. Then 3-((4-(4-amino-[1,4′-bipiperidin]-1′-yl)-3-fluorophenyl)amino)piperidine-2,6-dione (300 mg, 580 μmol, 1.00 eq, TFA) was added to the reaction mixture at 25 °C. The mixture was stirred at 25 °C for 1 h. LCMS showed 3-((4-(4-amino-[1,4′-bipiperidin]-1′-yl)-3-fluorophenyl)amino)piperidine-2,6-dione remained and the desired mass was detected. The reaction mixture was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 20%-50% B over 8.0 min) to give the title compound (Compound 532) (26.6 mg, 32.3 μmol, 5.58% yield, 97.2% purity) as a white solid. LCMS (ES⁺): m/z 800.5 [M+H]⁺.

### Example 543. Synthesis of trans-3-((3-fluoro-4-(4-((4-fluoro-1-(4-((5-fluoro-4-(3-(3-oxomorpholino)phenyl)pyrimidin-2-yl)amino)cyclohexane-1-carbonyl)piperidin-4-yl)methyl)piperazin-1-yl)phenyl)amino)piperidine-2,6-dione (Compound 530)

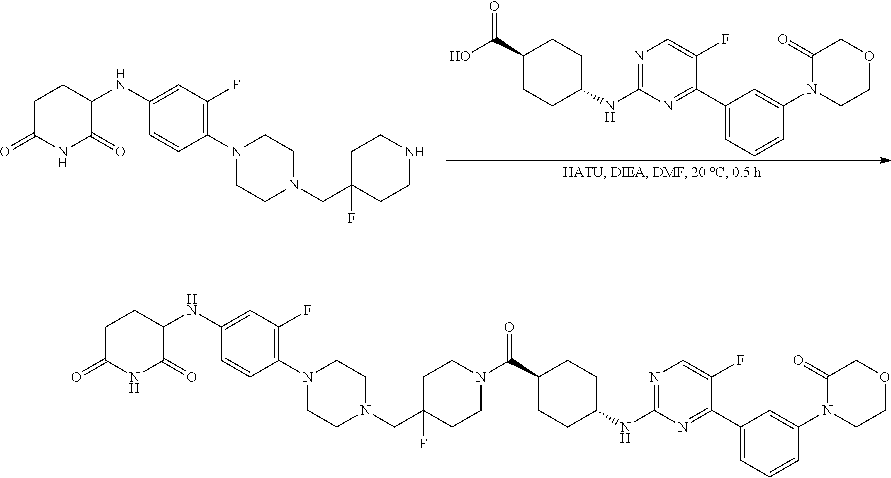 [SMILES: O=C1CCC(Nc2ccc(N3CCN(CC4(F)CCNCC4)CC3)c(F)c2)C(=O)N1 ; O=C1COCCN1c1cccc(-c2nc(N[C@H]3CC[C@H](C(=O)O)CC3)ncc2F)c1 ; O=C1CCC(Nc2ccc(N3CCN(CC4(F)CCN(C(=O)[C@H]5CC[C@H](Nc6ncc(F)c(-c7cccc(N8CCOCC8=O)c7)n6)CC5)CC4)CC3)c(F)c2)C(=O)N1]

Synthesis of trans-3-((3-fluoro-4-(4-((4-fluoro-1-(4-((5-fluoro-4-(3-(3-oxomorpholino)phenyl)pyrimidin-2-yl)amino)cyclohexane-1-carbonyl)piperidin-4-yl)methyl)piperazin-1-yl)phenyl)amino)piperidine-2,6-dione (Compound 530) To a solution of trans-4-((5-fluoro-4-(3-(3-oxomorpholino)phenyl)pyrimidin-2-yl)amino)cyclohexane-1-carboxylic acid (232 mg, 560 μmol, 0.600 eq) in DMF (8 mL) were added HATU (124 mg, 327 μmol, 0.350 eq) and DIEA (603 mg, 4.67 mmol, 813 μL, 5.00 eq) at 20 °C, then to the mixture was added 3-((3-fluoro-4-(4-((4-fluoropiperidin-4-yl)methyl)piperazin-1-yl)phenyl)amino)piperidine-2,6-dione (500 mg, 934 μmol, 1.00 eq, TFA) at 20 °C. The resulting mixture was stirred at 20 °C for 0.5 h. LCMS showed 3-((3-fluoro-4-(4-((4-fluoropiperidin-4-yl)methyl)piperazin-1-yl)phenyl)amino)piperidine-2,6-dione was consumed and the desired target MS was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 45%-75% B over 8.0 min) to give title compound (Compound 530) (90.1 mg, 101 mol, 10.8% yield, 91.6% purity) as a pale yellow solid. LCMS (ES⁺): m/z 818.2 [M+H]⁺

### Example 544. Synthesis of trans-3-((3-fluoro-4-(4-((1-(4-((5-fluoro-4-(3-(3-oxomorpholino)phenyl)pyrimidin-2-yl)amino)cyclohexane-1-carbonyl)piperidin-4-yl)methyl)piperazin-1-yl)phenyl)amino)piperidine-2,6-dione (Compound 553)

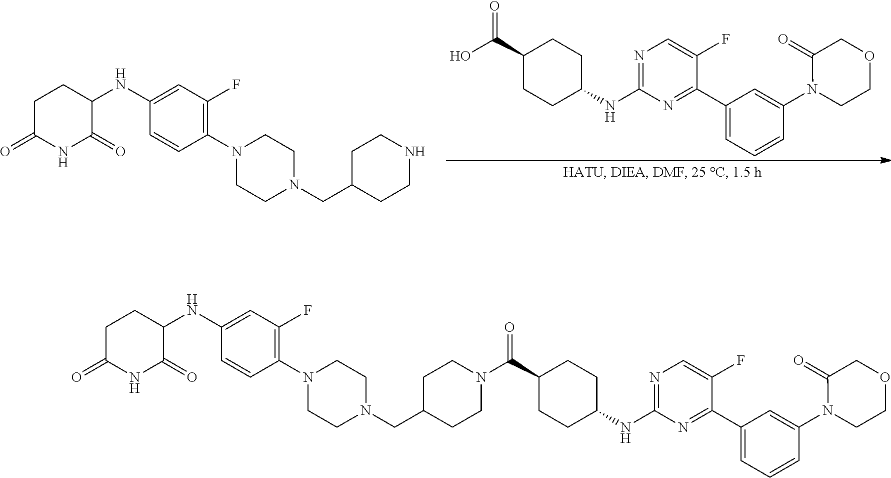 [SMILES: O=C1CCC(Nc2ccc(N3CCN(CC4CCNCC4)CC3)c(F)c2)C(=O)N1 ; O=C1COCCN1c1cccc(-c2nc(N[C@H]3CC[C@H](C(=O)O)CC3)ncc2F)c1 ; O=C1CCC(Nc2ccc(N3CCN(CC4CCN(C(=O)[C@H]5CC[C@H](Nc6ncc(F)c(-c7cccc(N8CCOCC8=O)c7)n6)CC5)CC4)CC3)c(F)c2)C(=O)N1]

Synthesis of trans-3-((3-fluoro-4-(4-((1-(4-((5-fluoro-4-(3-(3-oxomorpholino)phenyl)pyrimidin-2-yl)amino)cyclohexane-1-carbonyl)piperidin-4-yl)methyl)piperazin-1-yl)phenyl)amino)piperidine-2,6-dione (Compound 553). To a solution of trans-4-((5-fluoro-4-(3-(3-oxomorpholino)phenyl)pyrimidin-2-yl)amino)cyclohexane-1-carboxylic acid (247 mg, 595 μmol, 1.00 eq) in DMF (8 mL) were added DIEA (385 mg, 2.98 mmol, 518 μL, 5.00 eq) and HATU (272 mg, 714 μmol, 1.20 eq) at 25 °C. Then to the mixture was added 3-((3-fluoro-4-(4-(piperidin-4-ylmethyl)piperazin-1-yl)phenyl)amino)piperidine-2,6-dione (308 mg, 595 μmol, 1.00 eq, TFA) at 25 °C. The mixture was stirred at 25 °C for 1.5 h. LCMS showed 3-((3-fluoro-4-(4-(piperidin-4-ylmethyl)piperazin-1-yl)phenyl)amino)piperidine-2,6-dione was consumed and the desired mass was detected. The mixture was concentrated under reduced pressure to give a residue. This residue was purified by prep-HPLC (column: Phenomenex Luna C18 75*30 mm*3 um; mobile phase: [H₂O (0.2% FA)-ACN]; gradient: 1%-40% B over 8.0 min) to give the title compound (Compound 553) (201 mg, 232 μmol, 39.0% yield, 97.91% purity, FA) as a pale yellow solid. LCMS (ES⁺): m/z 800.4 [M+H]⁺

### Example 545. Synthesis of trans-3-((3-fluoro-4-(1-((1-(4-((5-fluoro-4-(3-(3-oxomorpholino)phenyl)pyrimidin-2-yl)amino)cyclohexane-1-carbonyl)piperidin-4-yl)methyl)piperidin-4-yl)phenyl)amino)piperidine-2,6-dione (Compound 552)

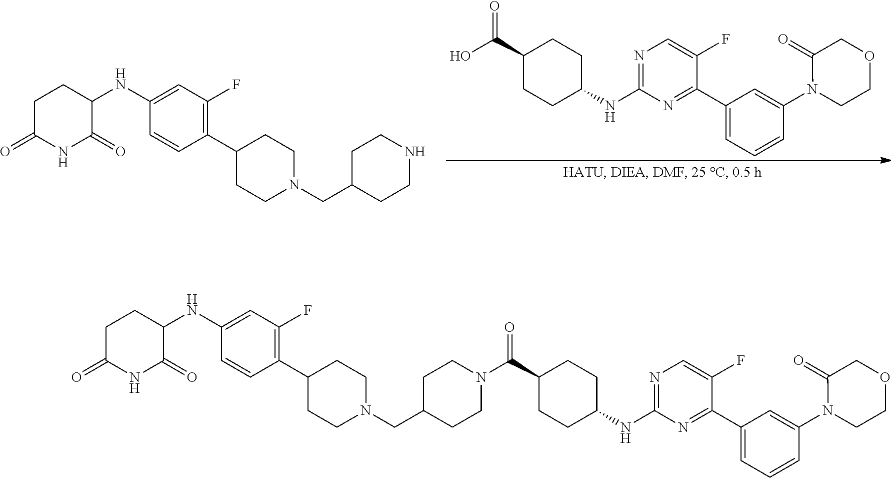 [SMILES: O=C1CCC(Nc2ccc(C3CCN(CC4CCNCC4)CC3)c(F)c2)C(=O)N1 ; O=C1COCCN1c1cccc(-c2nc(N[C@H]3CC[C@H](C(=O)O)CC3)ncc2F)c1 ; O=C1CCC(Nc2ccc(C3CCN(CC4CCN(C(=O)[C@H]5CC[C@H](Nc6ncc(F)c(-c7cccc(N8CCOCC8=O)c7)n6)CC5)CC4)CC3)c(F)c2)C(=O)N1]

Synthesis of trans-3-((3-fluoro-4-(1-((1-(4-((5-fluoro-4-(3-(3-oxomorpholino)phenyl)pyrimidin-2-yl)amino)cyclohexane-1-carbonyl)piperidin-4-yl)methyl)piperidin-4-yl)phenyl)amino)piperidine-2,6-dione (Compound 552). To a solution of trans-4-((5-fluoro-4-(3-(3-oxomorpholino)phenyl)pyrimidin-2-yl)amino)cyclohexane-1-carboxylic acid (263 mg, 635 μmol, 0.500 eq) in DMF (8 mL) were added HATU (145 mg, 381 μmol, 0.300 eq), DIEA (492 mg, 3.81 mmol, 663 μL, 3.00 eq) and 3-((3-fluoro-4-(1-(piperidin-4-ylmethyl)piperidin-4-yl)phenyl)amino)piperidine-2,6-dione (656 mg, 1.27 mmol, 1.00 eq, TFA) at 25 °C. The mixture was stirred at 25 °C for 0.5 h. LCMS showed 3-((3-fluoro-4-(1-(piperidin-4-ylmethyl)piperidin-4-yl)phenyl)amino)piperidine-2,6-dione was consumed completely and the desired mass was detected. The reaction mixture was concentrated under reduced pressure to give a residue. The residue was purified by prep-HPLC (column: Waters Xbridge BEH C18 250*50 mm*10 um; mobile phase: [H₂O (10 mM NH₄HCO₃)-ACN]; gradient: 30%-50% B over 10.0 min) to give the title compound (Compound 552) (75.3 mg, 90.2 μmol, 7.11% yield, 95.7% purity) as a white solid. LCMS (ES⁺): m/z 799.5 [M+H]⁺.

### Example 546. Synthesis of trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(3-oxomorpholino)phenyl)pyrimidin-2-yl)amino)cyclohexane-1-carboxamide (Compound 467)

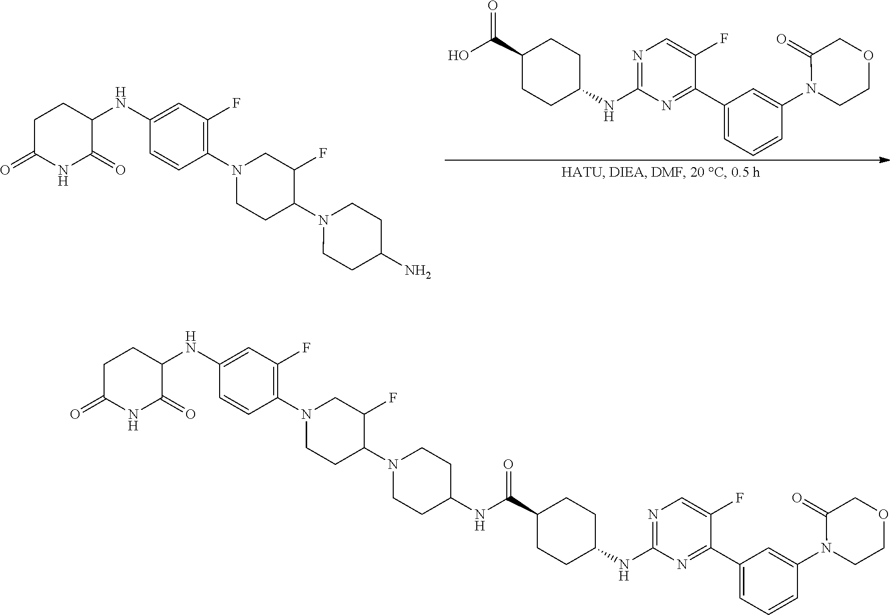 [SMILES: NC1CCN(C2CCN(c3ccc(NC4CCC(=O)NC4=O)cc3F)CC2F)CC1 ; O=C1COCCN1c1cccc(-c2nc(N[C@H]3CC[C@H](C(=O)O)CC3)ncc2F)c1 ; O=C1CCC(Nc2ccc(N3CCC(N4CCC(NC(=O)[C@H]5CC[C@H](Nc6ncc(F)c(-c7cccc(N8CCOCC8=O)c7)n6)CC5)CC4)C(F)C3)c(F)c2)C(=O)N1]

Synthesis of trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(3-oxomorpholino)phenyl)pyrimidin-2-yl)amino)cyclohexane-1-carboxamide (Compound 476). To a solution of trans-4-((5-fluoro-4-(3-(3-oxomorpholino)phenyl)pyrimidin-2-yl)amino)cyclohexane-1-carboxylic acid (54.2 mg, 131 μmol, 0.350 eq) in DMF (3 mL) were added HATU (49.7 mg, 131 μmol, 0.350 eq) and DIEA (242 mg, 1.87 mmol, 325 μL, 5.00 eq) at 20 °C, then 3-((4-(4-amino-3′-fluoro-[1,4′-bipiperidin]-1′-yl)-3-fluorophenyl)amino)piperidine-2,6-dione (200 mg, 373 μmol, 1.00 eq, TFA) was added to the mixture at 20 °C, the mixture was stirred at 20 °C for 0.5 h. LCMS showed 3-((4-(4-amino-3′-fluoro-[1,4′-bipiperidin]-1′-yl)-3-fluorophenyl)amino)piperidine-2,6-dione was consumed completely and the desired mass was detected. The reaction mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 10%-40% B over 8.0 min) to give the title compound (Compound 476) (50.5 mg, 55.7 μmol, 14.9% yield, 90.1% purity) as a brown solid. LCMS (ES⁺): m/z 818.4 [M+H]⁺

### Example 547. Synthesis of trans-N-(1′-(4-((2,6-dioxopiperidin-3-yl)amino)-2-fluorophenyl)-3′,3′-difluoro-[1,4′-bipiperidin]-4-yl)-4-((5-fluoro-4-(3-(3-oxomorpholino)phenyl)pyrimidin-2-yl)amino)cyclohexane-1-carboxamide (Compound 496)

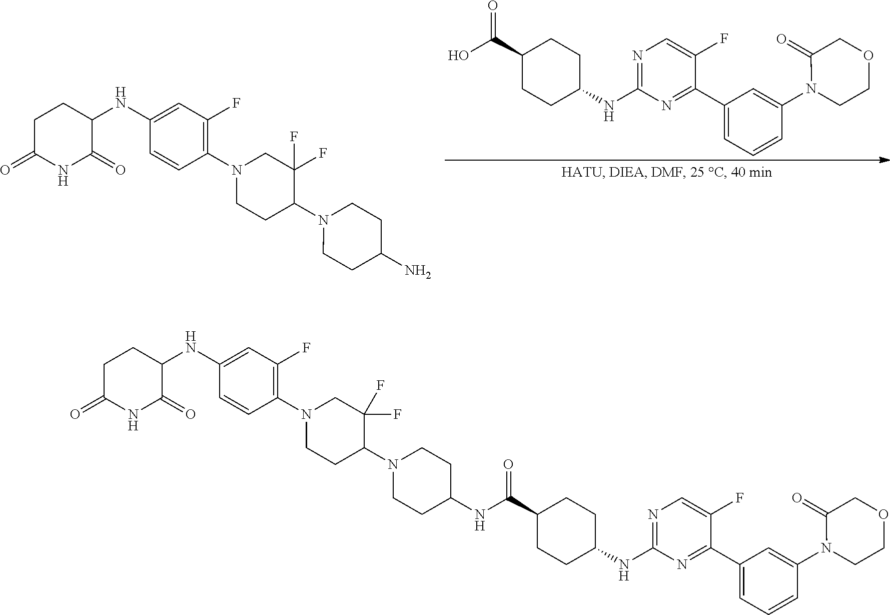 [SMILES: NC1CCN(C2CCN(c3ccc(NC4CCC(=O)NC4=O)cc3F)CC2(F)F)CC1 ; O=C1COCCN1c1cccc(-c2nc(N[C@H]3CC[C@H](C(=O)O)CC3)ncc2F)c1 ; O=C1CCC(Nc2ccc(N3CCC(N4CCC(NC(=O)[C@H]5CC[C@H](Nc6ncc(F)c(-c7cccc(N8CCOCC8=O)c7)n6)CC5)CC4)C(F)(F)C3)c(F)c2)C(=O)N1]

Synthesis of trans-N-(1′-(4-((2,6-dioxopiperidin-3-yl)amino)-2-fluorophenyl)-3′,3′-difluoro-[1,4′-bipiperidin]-4-yl)-4-((5-fluoro-4-(3-(3-oxomorpholino)phenyl)pyrimidin-2-yl)amino)cyclohexane-1-carboxamide (Compound 496). To a solution of trans-4-((5-fluoro-4-(3-(3-oxomorpholino)phenyl)pyrimidin-2-yl)amino)cyclohexane-1-carboxylic acid (59.9 mg, 145 μmol, 0.400 eq) in DMF (5.00 mL) were added DIEA (234 mg, 181 mmol, 315 μL, 5.00 eq) and HATU (48.1 mg, 126 μmol, 0.350 eq) at 25 °C. The mixture was stirred at 25 °C for 10 min. Then 3-((4-(4-amino-3′,3′-difluoro-[1,4′-bipiperidin]-1′-yl)-3-fluorophenyl)amino)piperidine-2,6-dione (200 mg, 361 μmol, 1.00 eq, TFA) was added to the reaction mixture at 25 °C. The mixture was stirred at 25 °C for 30 min. LCMS showed 3-((4-(4-amino-3′,3′-difluoro-[1,4′-bipiperidin]-1′-yl)-3-fluorophenyl)amino)piperidine-2,6-dione remained and the desired mass was detected. The mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 10%-50% B over 8.0 min) to give the title compound (Compound 496) (86.1 mg, 96.4 μmol, 26.7% yield, 93.6% purity) as a pale yellow solid. LCMS (ES⁺): m/z 836.4 [M+H]⁺.

### Example 548. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(2-oxo-2H-[1,2′-bipyridin]-6′-yl)pyrimidin-2-yl)amino)cyclohexyl)cyclohexane-1-carboxamide (Compound 567)

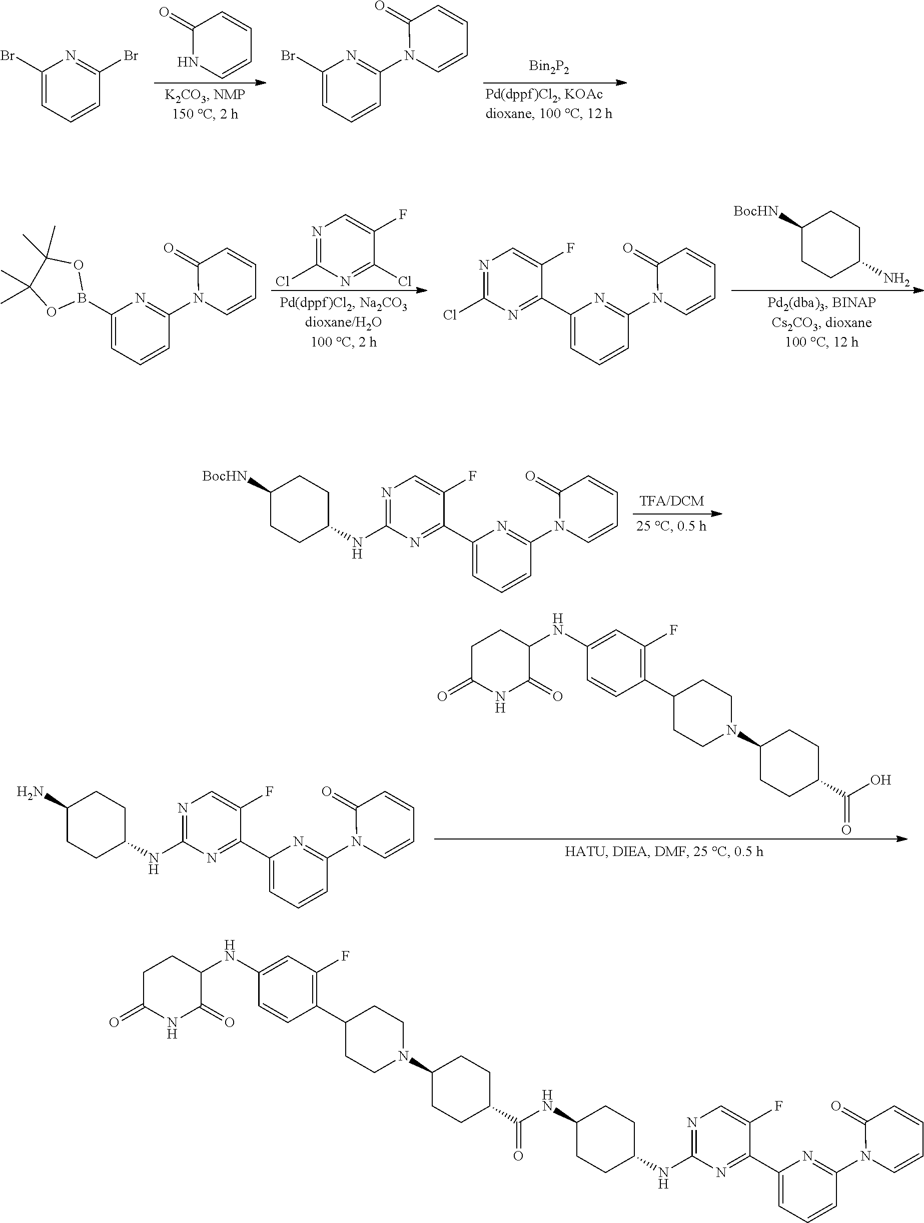

Synthesis of 6′-bromo-2H-[1,2′-bipyridin]-2-one. To a solution of 2,6-dibromopyridine (40.0 g, 169 mmol, 1.00 eq) and pyridin-2 (1H)-one (24.0 g, 254 mmol, 1.50 eq) in NMP (1000 mL) was added K₂CO₃ (35.0 g, 254 mmol, 1.50 eq) at 20 °C. The mixture was stirred at 150 °C for 2 h. LCMS showed the desired mass was detected. To the mixture was added H₂O (1000 mL) and the mixture was extracted with EtOAc (1000 mL*3). The combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (40.0 g, 148 mmol, 87.8% yield) as a white solid. LCMS (ES⁺): m/z 251.1 [M+H]⁺

Synthesis of 6′-(4,4,5,5-tetramethyl-1,3,2-dioxaborolan-2-yl)-2H-[1,2′-bipyridin]-2-one. A mixture of 6′-bromo-2H-[1,2′-bipyridin]-2-one (15.0 g, 59.8 mmol, 1.00 eq), 4,4,5,5-tetramethyl-2-(4,4,5,5-tetramethyl-1,3,2-dioxaborolan-2-yl)-1,3,2-dioxaborolane (18.2 g, 71.8 mmol, 1.20 eq), KOAc (17.6 g, 179 mmol, 3.00 eq) and Pd(dppf)Cl₂ (4.38 g, 5.98 mmol, 0.100 eq) in dioxane (500 mL) was stirred at 100 °C for 12 h under N₂ atmosphere. LCMS showed 6′-bromo-2H-[1,2′-bipyridin]-2-one was consumed and the desired mass was detected. The mixture was concentrated under reduced pressure to give the title compound (20.0 g, crude) as a black oil. LCMS (ES⁺): m/z 299.1 [M+H]⁺

Synthesis of 6′-(2-chloro-5-fluoropyrimidin-4-yl)-2H-[1,2′-bipyridin]-2-one. A mixture of 6′-(4,4,5,5-tetramethyl-1,3,2-dioxaborolan-2-yl)-2H-[1,2′-bipyridin]-2-one (15.0 g, 50.4 mmol, 1.00 eq), 2,4-dichloro-5-fluoropyrimidine (8.40 g, 50.4 mmol, 1.00 eq), Na₂CO₃ (10.7 g, 101 mmol, 2.00 eq), Pd(dppf)Cl₂ (1.84 g, 2.52 mmol, 0.0500 eq) in dioxane (300 mL) and H₂O (30 mL) was stirred at 100 °C for 2 h under N₂ atmosphere. LCMS showed 6′-(4,4,5,5-tetramethyl-1,3,2-dioxaborolan-2-yl)-2H-[1,2′-bipyridin]-2-one was consumed and the desired mass was detected. To the mixture was added H₂O (300 mL) and the mixture was extracted with EtOAc (300 mL*3). The combined organic layers were dried over Na₂SO₄, filtered and the filtrate was concentrated under reduced pressure to give a residue. The residue was purified by MPLC (SiO₂, PE/EtOAc=1/0 to 5/1) to give the title compound (20.0 g, crude) as a yellow oil. LCMS (ES⁺): m/z 303.1 [M+H]⁺

Synthesis of trans-tert-butyl (4-((5-fluoro-4-(2-oxo-2H-[1,2′-bipyridin]-6′-yl)pyrimidin-2-yl)amino)cyclohexyl)carbamate. To a solution of 6′-(2-chloro-5-fluoropyrimidin-4-yl)-2H-[1,2′-bipyridin]-2-one (2.50 g, 8.25 mmol, 1.00 eq) and trans-tert-butyl (4-aminocyclohexyl)carbamate (1.77 g, 8.25 mmol, 1.00 eq) in dioxane (150 mL) were added Cs₂CO₃ (8.05 g, 24.8 mmol, 3.00 eq), BINAP (360 mg, 580 μmol, 0.0700 eq) and Pd₂(dba)₃ (378 mg, 413 μmol, 0.0500 eq) at 20 °C under N₂ atmosphere, the mixture was stirred at 100 °C for 12 h under N₂ atmosphere. LCMS showed 6′-(2-chloro-5-fluoropyrimidin-4-yl)-2H-[1,2′-bipyridin]-2-one was consumed and the desired target MS was detected. H₂O (200 mL) was added to the solution and the mixture was extracted with EtOAc (200 mL*3). Then the combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (830 mg, 1.73 mmol, 20.9% yield) as a yellow solid. LCMS (ES⁺): m/z 481.2 [M+H]⁺

Synthesis of trans-6′-(2-((4-aminocyclohexyl)amino)-5-fluoropyrimidin-4-yl)-2H-[1,2′-bipyridin]-2-one. A solution of trans-tert-butyl (4-((5-fluoro-4-(2-oxo-2H-[1,2′-bipyridin]-6′-yl)pyrimidin-2-yl)amino)cyclohexyl)carbamate (121 mg, 252 μmol, 1.00 eq) in DCM (3 mL) and TFA (1 mL) was stirred at 25 °C for 0.5 h. LCMS showed trans-tert-butyl (4-((5-fluoro-4-(2-oxo-2H-[1,2′-bipyridin]-6′-yl)pyrimidin-2-yl)amino)cyclohexyl)carbamate was consumed and the desired target MS was detected. The mixture was concentrated under reduced pressure to give the title compound (121 mg, crude, TFA) as a yellow oil. LCMS (ES⁺): m/z 381.3 [M+H]⁺

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(2-oxo-2H-[1,2′-bipyridin]-6′-yl)pyrimidin-2-yl)amino)cyclohexyl)cyclohexane-1-carboxamide (Compound 567). To a solution of trans-4-(4-(4-((2,6-dioxopiperidin-3-yl)amino)-2-fluorophenyl)piperidin-1-yl)cyclohexane-1-carboxylic acid (265 mg, 614 μmol, 1.00 eq) in DMF (5 mL) were added HATU (81.7 mg, 215 μmol, 0.350 eq), DIEA (397 mg, 3.07 mmol, 535 μL, 5.00 eq) and trans-6′-(2-((4-aminocyclohexyl)amino)-5-fluoropyrimidin-4-yl)-2H-[1,2′-bipyridin]-2-one (118 mg, 240 μmol, 0.390 eq, TFA) at 25 °C, the mixture was stirred at 25 °C for 0.5 h. LCMS showed trans-6′-(2-((4-aminocyclohexyl)amino)-5-fluoropyrimidin-4-yl)-2H-[1,2′-bipyridin]-2-one was consumed and the desired target MS was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 24%-51% B over 8.0 min) to give the title compound (Compound 567) (35.1 mg, 43.2 μmol, 7.04% yield, 97.8% purity) as a white solid. LCMS (ES⁺): m/z 794.4 [M+H]⁺

### Example 549. Synthesis of trans-N-(trans-4-(4-(4-((2,6-dioxopiperidin-3-yl)amino)-2-fluorophenyl)piperidin-1-yl)cyclohexyl)-4-((5-fluoro-4-(2-oxo-2H-[1,2′-bipyridin]-6′-yl)pyrimidin-2-yl)amino)cyclohexane-1-carboxamide (Compound 520)

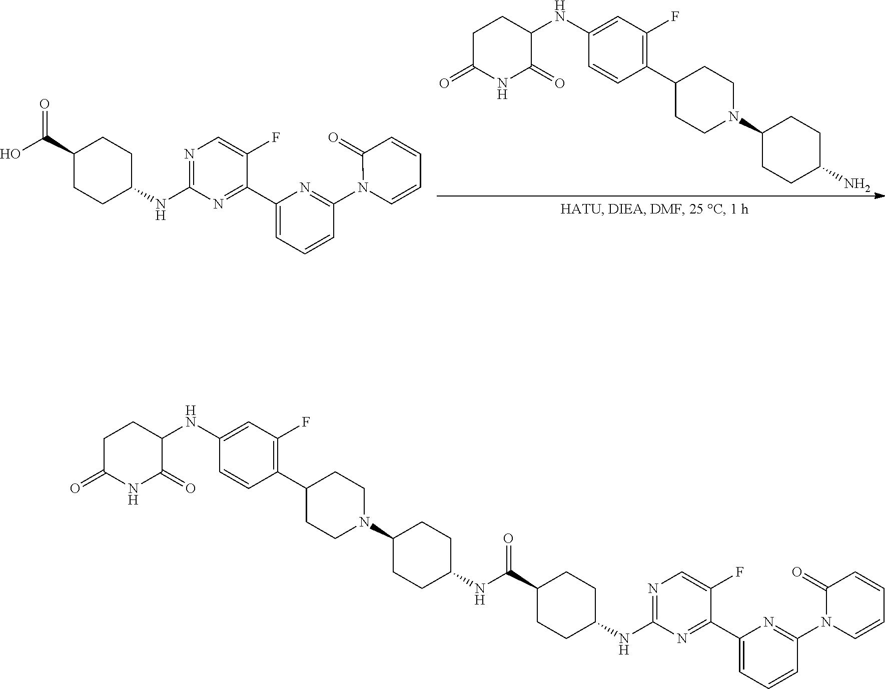 [SMILES: O=c1ccccn1-c1cccc(-c2nc(N[C@H]3CC[C@H](C(=O)O)CC3)ncc2F)n1 ; N[C@H]1CC[C@H](N2CCC(c3ccc(NC4CCC(=O)NC4=O)cc3F)CC2)CC1 ; O=C1CCC(Nc2ccc(C3CCN([C@H]4CC[C@H](NC(=O)[C@H]5CC[C@H](Nc6ncc(F)c(-c7cccc(-n8ccccc8=O)n7)n6)CC5)CC4)CC3)c(F)c2)C(=O)N1]

Synthesis of trans-N-(trans-4-(4-(4-((2,6-dioxopiperidin-3-yl)amino)-2-fluorophenyl)piperidin-1-yl)cyclohexyl)-4-((5-fluoro-4-(2-oxo-2H-[1,2′-bipyridin]-6′-yl)pyrimidin-2-yl)amino)cyclohexane-1-carboxamide (Compound 520). To a solution of trans-4-((5-fluoro-4-(2-oxo-2H-[1,2′-bipyridin]-6′-yl)pyrimidin-2-yl)amino)cyclohexane-1-carboxylic acid (300 mg, 732 μmol, 1.00 eq, TFA) and 3-[4-[1-(trans-4-aminocyclohexyl)-4-piperidyl]-3-fluoro-anilino]piperidine-2,6-dione (294 mg, 732 μmol, 1.00 eq, TFA) in DMF (5 mL) at 25 °C was added DIEA (189 mg, 1.47 mmol, 255 μL, 2.00 eq) and HATU (557 mg, 1.47 mmol, 2.00 eq) at 25 °C. The mixture was stirred at 25 °C for 1 h. LC-MS showed the desired mass was detected. The reaction mixture was filtered and concentrated under reduced pressure. The residue was purified by prep-HPLC (column: Waters Xbridge BEH C18 100*30 mm*10 um; mobile phase: [H₂O (10 mM NH₄HCO₃)-ACN]; gradient: 20%-50% B over 8.0 min) to give the title compound (Compound 520) (11.4 mg, 14.3 μmol, 0.980% yield, 100% purity) as a pale yellow solid. LCMS (ES⁺): m/z 794.4 [M+H]⁺.

### Example 550. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(6-(2-oxo-1,3-oxazinan-3-yl)pyridin-2-yl)pyrimidin-2-yl)amino)cyclohexyl)cyclohexane-1-carboxamide (Compound 539)

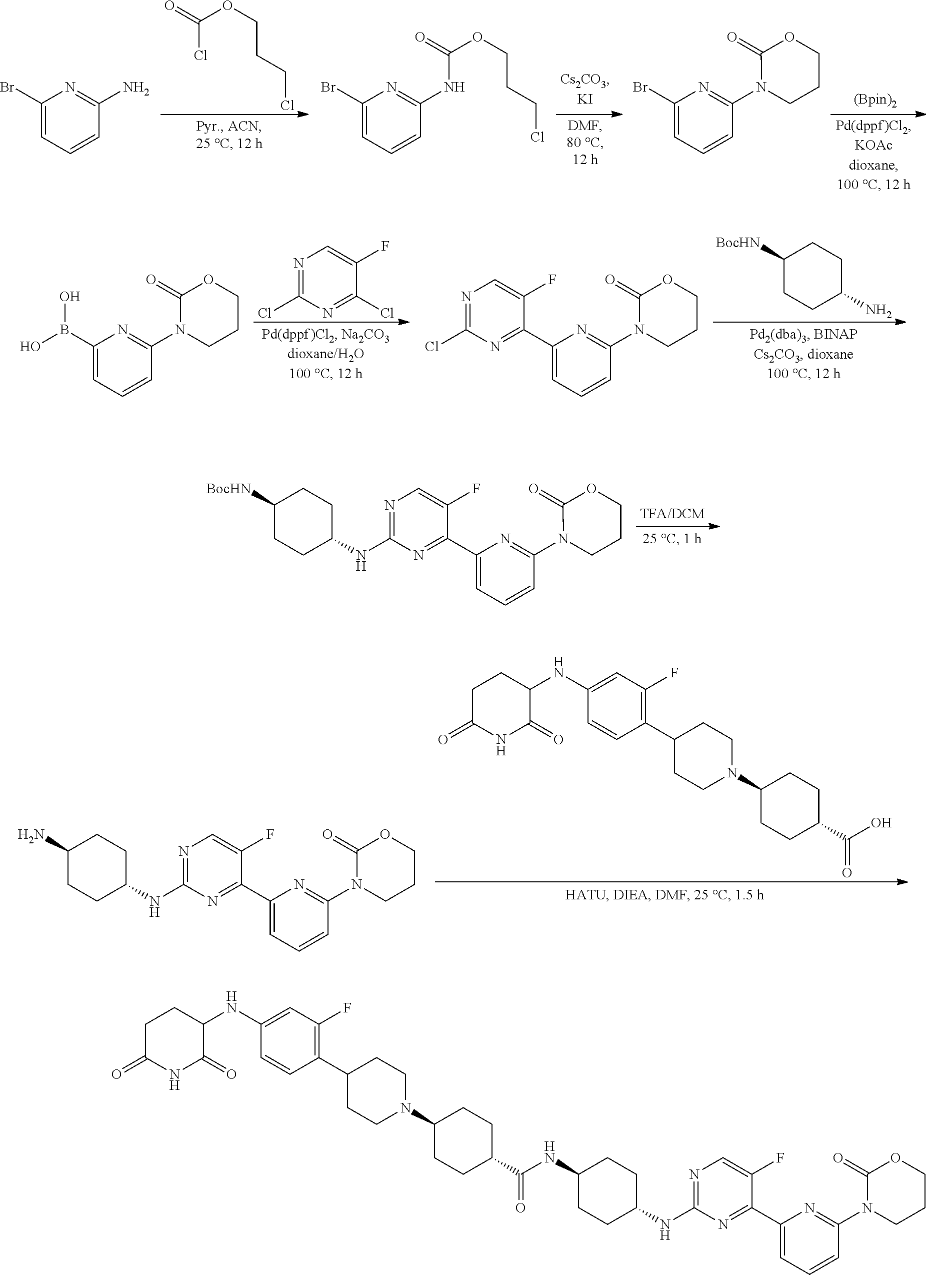

Synthesis of 3-chloropropyl (6-bromopyridin-2-yl)carbamate. To a solution of 6-bromopyridin-2-amine (27.0 g, 156 mmol, 1.00 eq) in ACN (500 mL) were added pyridine (24.6 g, 312 mmol, 25.2 mL, 2.00 eq) and 3-chloropropyl carbonochloridate (24.5 g, 156 mmol, 18.8 mL, 1.00 eq) at 25 °C. The mixture was stirred at 25 °C for 12 h. LCMS showed the desired mass was detected. The mixture was diluted with water (500 mL) and extracted with EtOAc (300 mL*3). The combined organic layers were washed with brine (300 mL*3), dried over Na₂SO₄, filtered and the filtrate was concentrated under reduced pressure to give the title compound (48.0 g, crude) as a yellow oil. LCMS (ES⁺): m/z 292.9 [M+H]⁺.

Synthesis of 3-(6-bromopyridin-2-yl)-1,3-oxazinan-2-one. To a solution of 3-chloropropyl (6-bromopyridin-2-yl)carbamate (46.0 g, 157 mmol, 1.00 eq) in DMF (1500 mL) were added Cs₂CO₃ (102 g, 312 mmol, 2.00 eq) and KI (52.0 g, 312 mmol, 2.00 eq) at 25 °C. The mixture was stirred at 80 °C for 12 h. LCMS showed 3-chloropropyl (6-bromopyridin-2-yl)carbamate was consumed completely and the desired mass was detected. The mixture was diluted with water (1000 mL) and extracted with EtOAc (500 mL*3). The combined organic layers were washed with brine (500 mL*3), dried over Na₂SO₄, filtered and the filtrate was concentrated under reduced pressure to give a residue. The residue was purified by MPLC (SiO₂, EtOAc/THF=3/1 to 0/1) to give the title compound (40.0 g, 62.2 mmol, 39.7% yield) as a yellow oil. LCMS (ES⁺): m/z 257.0 [M+H]⁺

Synthesis of (6-(2-oxo-1,3-oxazinan-3-yl)pyridin-2-yl)boronic acid. A mixture of 3-(6-bromopyridin-2-yl)-1,3-oxazinan-2-one (20.0 g, 77.8 mmol, 1.00 eq), 4,4,5,5-tetramethyl-2-(4,4,5,5-tetramethyl-1,3,2-dioxaborolan-2-yl)-1,3,2-dioxaborolane (19.7 g, 77.8 mmol, 1.00 eq), Pd(dppf)Cl₂ (5.69 g, 7.78 mmol, 0.100 eq) and KOAc (22.9 g, 233 mmol, 3.00 eq) in 1,4-dioxane (500 mL) was degassed and purged with N₂ at 25 °C for three times and then the mixture was stirred at 100 °C for 2 h under N₂ atmosphere. LCMS showed 3-(6-bromopyridin-2-yl)-1,3-oxazinan-2-one was consumed completely and the desired mass was detected. The mixture was concentrated under reduced pressure to give the title compound (20.0 g, crude) as a brown oil. LCMS (ES⁺): m/z 223.1 [M+H]⁺.

Synthesis of 3-(6-(2-chloro-5-fluoropyrimidin-4-yl)pyridin-2-yl)-1,3-oxazinan-2-one. A mixture of (6-(2-oxo-1,3-oxazinan-3-yl)pyridin-2-yl)boronic acid (20.0 g, 90.1 mmol, 1.00 eq), 2,4-dichloro-5-fluoropyrimidine (15.0 g, 90.1 mmol, 1.00 eq), Na₂CO₃ (28.7 g, 270 mmol, 3.00 eq) and Pd(dppf)Cl₂ (6.59 g, 9.01 mmol, 0.100 eq) in 1,4-dioxane (600 mL) and H₂O (120 mL) was degassed and purged with N₂ at 25 °C for three times and then the mixture was stirred at 100 °C for 12 h under N₂ atmosphere. LCMS showed (6-(2-oxo-1,3-oxazinan-3-yl)pyridin-2-yl)boronic acid was consumed completely and the desired mass was detected. The mixture was concentrated under reduced pressure to give a residue. To the residue was added H₂O (300 mL), and then the mixture was extracted with EtOAc (300 mL*3). The combined organic layers were washed with brine (500 mL*3), dried over Na₂SO₄, filtered and the filtrate was concentrated under reduced pressure to give a residue. The residue was purified by MPLC (SiO₂, PE/EtOAc=1/0 to 1/1) to give the title compound (10.0 g, 30.3 mmol, 36.0% yield) as a yellow solid. LCMS (ES⁺): m/z 309.1 [M+H]⁺.

Synthesis of trans-tert-butyl (4-((5-fluoro-4-(6-(2-oxo-1,3-oxazinan-3-yl)pyridin-2-yl)pyrimidin-2-yl)amino)cyclohexyl)carbamate. To a solution of 3-(6-(2-chloro-5-fluoropyrimidin-4-yl)pyridin-2-yl)-1,3-oxazinan-2-one (1.90 g, 6.15 mmol, 1.00 eq) and trans-tert-butyl (4-aminocyclohexyl)carbamate (1.32 g, 6.15 mmol, 1.00 eq) in dioxane (70 mL) were added BINAP (383 mg, 616 μmol, 0.100 eq), Cs₂CO₃ (4.01 g, 12.3 mmol, 2.00 eq) and Pd₂(dba)₃ (564 mg, 616 μmol, 0.100 eq) at 25 °C under N₂ atmosphere. The mixture was stirred at 100 °C for 12 h under N₂ atmosphere. LCMS showed the 3-(6-(2-chloro-5-fluoropyrimidin-4-yl)pyridin-2-yl)-1,3-oxazinan-2-one was consumed completely and the desired mass was detected. H₂O (70 mL) was added to the mixture and then the mixture was extracted with EtOAc (50 mL*3). The combined organic layers were washed with brine (50 mL*3), dried over Na₂SO₄, filtered and the filtrate was concentrated under reduced pressure to give a residue. The residue was purified by MPLC (SiO₂, PE/EtOAc=1/0 to 1/1) to give the title compound (1.03 g, 2.12 mmol, 34.4% yield) as a yellow oil. LCMS (ESI+): m/s 487.2 [M+H]⁺.

Synthesis of trans-3-(6-(2-((4-aminocyclohexyl)amino)-5-fluoropyrimidin-4-yl)pyridin-2-yl)-1,3-oxazinan-2-one. A solution of trans-tert-butyl (4-((5-fluoro-4-(6-(2-oxo-1,3-oxazinan-3-yl)pyridin-2-yl)pyrimidin-2-yl)amino)cyclohexyl)carbamate (120 mg, 247 μmol, 1.00 eq) in DCM (2 mL) and TFA (0.5 mL) was stirred at 25 °C for 1 h. LCMS showed that trans-tert-butyl (4-((5-fluoro-4-(6-(2-oxo-1,3-oxazinan-3-yl)pyridin-2-yl)pyrimidin-2-yl)amino)cyclohexyl)carbamate was consumed and the desired mass was detected. The mixture was concentrated under reduced pressure to give the title compound (120 mg, crude, TFA) as a yellow oil. LCMS (ESI+): m/s 387.1 [M+H]⁺.

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(6-(2-oxo-1,3-oxazinan-3-yl)pyridin-2-yl)pyrimidin-2-yl)amino)cyclohexyl)cyclohexane-1-carboxamide (Compound 539). To a solution of trans-4-(4-(4-((2,6-dioxopiperidin-3-yl)amino)-2-fluorophenyl)piperidin-1-yl)cyclohexane-1-carboxylic acid (250 mg, 579 μmol, 1.00 eq) in DMF (5 mL) were added HATU (77.1 mg, 203 μmol, 0.350 eq) and DIEA (383 mg, 2.90 mmol, 515 μL, 5.00 eq) at 25 °C, then trans-3-(6-(2-((4-aminocyclohexyl)amino)-5-fluoropyrimidin-4-yl)pyridin-2-yl)-1,3-oxazinan-2-one (116 mg, 232 μmol, 0.400 eq, TFA) was added to the mixture, the mixture was stirred at 25 °C for 1 h. LCMS showed trans-3-(6-(2-((-4-aminocyclohexyl)amino)-5-fluoropyrimidin-4-yl)pyridin-2-yl)-1,3-oxazinan-2-one remained and desired target MS was detected. Then HATU (55.1 mg, 145 μmol, 0.250 eq) was added to the mixture at 25 °C, the mixture was stirred at 25 °C for another 0.5 h. LCMS showed trans-3-(6-(2-((-4-aminocyclohexyl)amino)-5-fluoropyrimidin-4-yl)pyridin-2-yl)-1,3-oxazinan-2-one was consumed completely and the desired target MS was detected. The mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 10%-50% B over 8.0 min) to give the title compound (Compound 539) (37.5 mg, 40.5 μmol, 7.00% yield, 91.5% purity, FA) as a white solid. LCMS (ES+): m/z 800.4 [M+H]⁺.

### Example 551. Synthesis of (trans)-N-((trans)-4-(4-(4-((2,6-dioxopiperidin-3-yl)amino)-2-fluorophenyl)piperidin-1-yl)cyclohexyl)-4-((5-fluoro-4-(6-(2-oxo-1,3-oxazinan-3-yl)pyridin-2-yl)pyrimidin-2-yl)amino)cyclohexane-1-carboxamide (Compound 519)

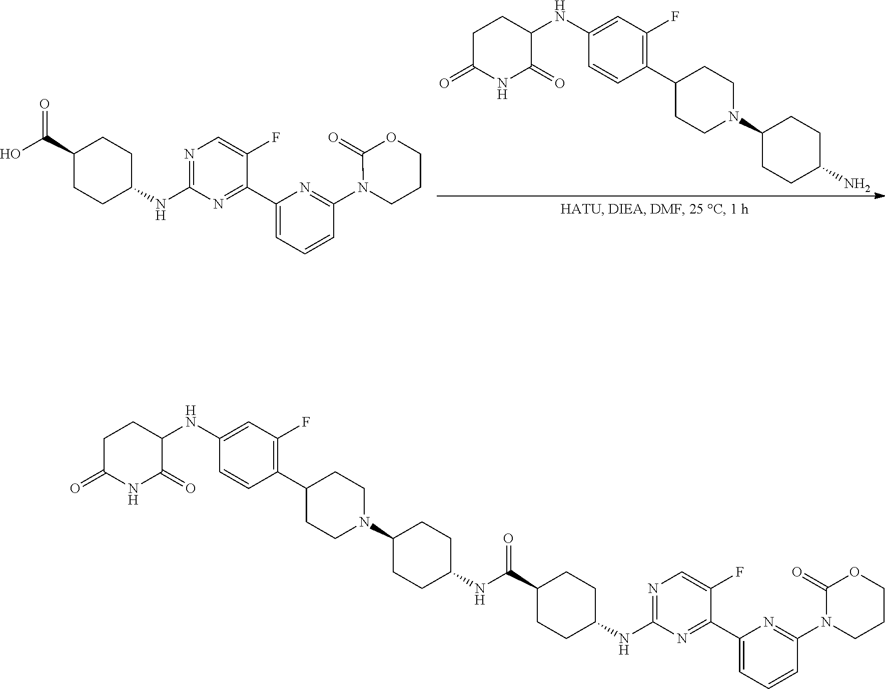 [SMILES: O=C1OCCCN1c1cccc(-c2nc(N[C@H]3CC[C@H](C(=O)O)CC3)ncc2F)n1 ; N[C@H]1CC[C@H](N2CCC(c3ccc(NC4CCC(=O)NC4=O)cc3F)CC2)CC1 ; O=C1CCC(Nc2ccc(C3CCN([C@H]4CC[C@H](NC(=O)[C@H]5CC[C@H](Nc6ncc(F)c(-c7cccc(N8CCCOC8=O)n7)n6)CC5)CC4)CC3)c(F)c2)C(=O)N1]

Synthesis of (trans)-N-((trans)-4-(4-(4-((2,6-dioxopiperidin-3-yl)amino)-2-fluorophenyl)piperidin-1-yl)cyclohexyl)-4-((5-fluoro-4-(6-(2-oxo-1,3-oxazinan-3-yl)pyridin-2-yl)pyrimidin-2-yl)amino)cyclohexane-1-carboxamide (Compound 519). To a solution of trans-4-[[5-fluoro-4-[6-(2-oxo-1,3-oxazinan-3-yl)-2-pyridyl]pyrimidin-2-yl]amino]cyclohexanecarboxylic acid (270 mg, 509 μmol, 1.00 eq, TFA) and trans-3-((4-(1-(4-aminocyclohexyl)piperidin-4-yl)-3-fluorophenyl)amino)piperidine-2,6-dione (205 mg, 397 mol, 0.78 eq, TFA) in DMF (3 mL) was added HATU (290 mg, 764 μmol, 1.50 eq) and DIEA (197 mg, 1.53 mmol, 266 μL, 3.00 eq) at 25 °C. The mixture was stirred at 25 °C for 1 h. LC-MS showed trans-4-[[5-fluoro-4-[6-(2-oxo-1,3-oxazinan-3-yl)-2-pyridyl]pyrimidin-2-yl]amino]cyclohexanecarboxylic acid was consumed completely and the desired mass was detected. The reaction mixture was concentrated under reduced pressure. The residue was purified by prep-HPLC (column: Phenomenex Luna C18 100*30 mm*3 um; mobile phase: [H₂O (0.2% FA)-ACN]; gradient: 1%-40% B over 8.0 min) to give the title compound (Compound 519) (70.8 mg, 82.9 μmol, 16.2% yield, 96.3% purity, 0.5 FA) as a white solid. LCMS (ES⁺): m/z 800.4 [M+H]⁺.

### Example 552. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(6-(2-oxopiperidin-1-yl)pyridin-2-yl)pyrimidin-2-yl)amino)cyclohexyl)cyclohexane-1-carboxamide (Compound 566)

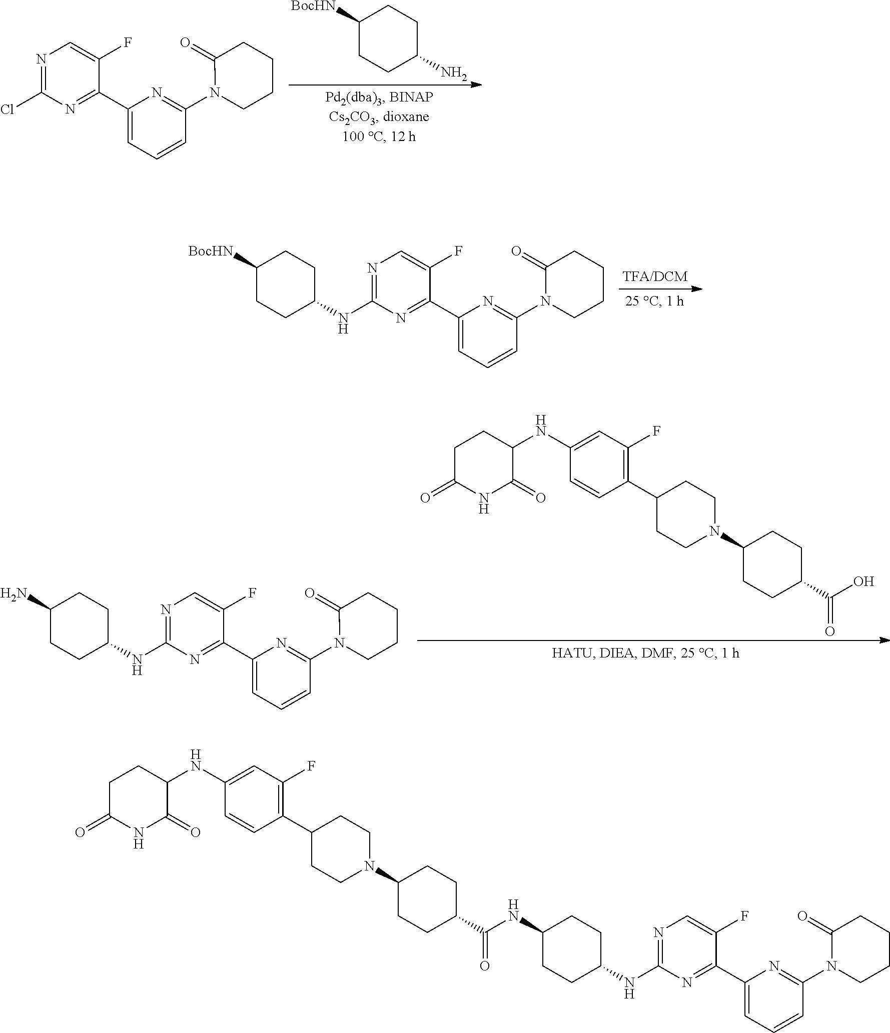 [SMILES: O=C1CCCCN1c1cccc(-c2nc(Cl)ncc2F)n1 ; CC(C)(C)OC(=O)N[C@H]1CC[C@H](N)CC1 ; CC(C)(C)OC(=O)N[C@H]1CC[C@H](Nc2ncc(F)c(-c3cccc(N4CCCCC4=O)n3)n2)CC1 ; N[C@H]1CC[C@H](Nc2ncc(F)c(-c3cccc(N4CCCCC4=O)n3)n2)CC1 ; O=C1CCC(Nc2ccc(C3CCN([C@H]4CC[C@H](C(=O)O)CC4)CC3)c(F)c2)C(=O)N1 ; O=C1CCC(Nc2ccc(C3CCN([C@H]4CC[C@H](C(=O)N[C@H]5CC[C@H](Nc6ncc(F)c(-c7cccc(N8CCCCC8=O)n7)n6)CC5)CC4)CC3)c(F)c2)C(=O)N1]

Synthesis of trans-tert-butyl (4-((5-fluoro-4-(6-(2-oxopiperidin-1-yl)pyridin-2-yl)pyrimidin-2-yl)amino)cyclohexyl)carbamate. To a solution of 1-(6-(2-chloro-5-fluoropyrimidin-4-yl)pyridin-2-yl)piperidin-2-one (3.00 g, 9.78 mmol, 1.00 eq) and trans-tert-butyl (4-aminocyclohexyl)carbamate (4.19 g, 19.6 mmol, 2.00 eq) in dioxane (100 mL) were added BINAP (609 mg, 978 μmol, 0.100 eq), Cs₂CO₃ (6.37 g, 19.6 mmol, 2.00 eq) and Pd₂(dba)₃ (895.65 mg, 978.08 μmol, 0.100 eq) at 20 °C under N₂. The mixture was stirred at 100 °C for 12 h under N₂. LCMS showed 1-(6-(2-chloro-5-fluoropyrimidin-4-yl)pyridin-2-yl)piperidin-2-one was consumed completely and the desired mass was detected. H₂O (100 mL) was added to the mixture, then the mixture was extracted with EtOAc (50 mL*3). The combined organic layers were dried over Na₂SO₄, filtered and the filtrate was concentrated under reduced pressure to give a residue. The residue was purified by MPLC (SiO₂, PE/EtOAc=1/0 to 1/1) to give the title compound (1.64 g, 3.38 mmol, 34.6% yield) as a yellow solid. LCMS (ES+): m/z 485.2 [M+H]⁺

Synthesis of trans-1-(6-(2-((4-aminocyclohexyl)amino)-5-fluoropyrimidin-4-yl)pyridin-2-yl)piperidin-2-one. A solution of trans-tert-butyl (4-((5-fluoro-4-(6-(2-oxopiperidin-1-yl)pyridin-2-yl)pyrimidin-2-yl)amino)cyclohexyl)carbamate (210 mg, 433 μmol, 1.00 eq) in DCM (1 mL) and TFA (1 mL) was stirred at 25 °C for 0.5 h. LCMS showed trans-tert-butyl (4-((5-fluoro-4-(6-(2-oxopiperidin-1-yl)pyridin-2-yl)pyrimidin-2-yl)amino)cyclohexyl)carbamate was consumed completely and the desired mass was detected. The mixture was concentrated under reduced pressure to give the title compound (216 mg, crude, TFA) as a yellow oil. LCMS (ES+): m/z 385.2 [M+H]⁺

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(6-(2-oxopiperidin-1-yl)pyridin-2-yl)pyrimidin-2-yl)amino)cyclohexyl)cyclohexane-1-carboxamide (Compound 566). To a stirred solution of trans-4-(4-(4-((2,6-dioxopiperidin-3-yl)amino)-2-fluorophenyl)piperidin-1-yl)cyclohexane-1-carboxylic acid (265 mg, 614 μmol, 1.00 eq) and DIEA (398 mg, 3.08 mmol, 535 μL, 5.00 eq) in DMF (3 mL) were added HATU (81.7 mg, 215 μmol, 0.350 eq) and trans-1-(6-(2-((4-aminocyclohexyl)amino)-5-fluoropyrimidin-4-yl)pyridin-2-yl)piperidin-2-one (122 mg, 246 μmol, 0.400 eq, TFA) at 25 °C. The resulting mixture was stirred at 25 °C for 1 h. LCMS showed trans-4-(4-(4-((2,6-dioxopiperidin-3-yl)amino)-2-fluorophenyl)piperidin-1-yl)cyclohexane-1-carboxylic acid was consumed completely and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 35%-65% B over 8.0 min) to give the title compound (Compound 566) (12.4 mg, 14.9 μmol, 2.43% yield, 95.9% purity) as a white solid. LCMS (ES+): m/z 798.4 [M+H]⁺

### Example 553. Synthesis of trans-N-(trans-4-(4-(4-((2,6-dioxopiperidin-3-yl)amino)-2-fluorophenyl)piperidin-1-yl)cyclohexyl)-4-((5-fluoro-4-(6-(2-oxopiperidin-1-yl)pyridin-2-)pyrimidin-2-yl)amino)cyclohexane-1-carboxamide (Compound 546)

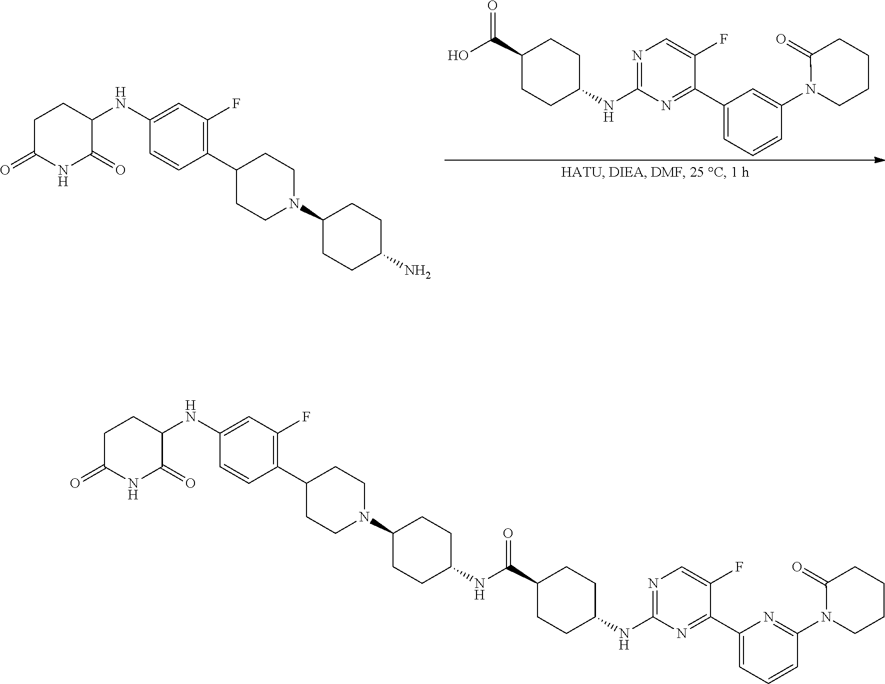 [SMILES: N[C@H]1CC[C@H](N2CCC(c3ccc(NC4CCC(=O)NC4=O)cc3F)CC2)CC1 ; O=C1CCCCN1c1cccc(-c2nc(N[C@H]3CC[C@H](C(=O)O)CC3)ncc2F)c1 ; O=C1CCC(Nc2ccc(C3CCN([C@H]4CC[C@H](NC(=O)[C@H]5CC[C@H](Nc6ncc(F)c(-c7cccc(N8CCCCC8=O)n7)n6)CC5)CC4)CC3)c(F)c2)C(=O)N1]

Synthesis of trans-N-(trans-4-(4-(4-((2,6-dioxopiperidin-3-yl)amino)-2-fluorophenyl)piperidin-1-yl)cyclohexyl)-4-((5-fluoro-4-(6-(2-oxopiperidin-1-yl)pyridin-2-yl)pyrimidin-2-yl)amino)cyclohexane-1-carboxamide (Compound 546). To a solution of trans-3-[4-[1-(4-aminocyclohexyl)-4-piperidyl]-3-fluoro-anilino]piperidine-2,6-dione (270 mg, 670 μmol, 1.00 eq) in DMF (1.5 mL) was added DIEA (260 mg, 2.01 mmol, 350 μL, 3.00 eq) until pH-7. To a solution of trans-4-[[5-fluoro-4-[6-(2-oxo-1-piperidyl)-2-pyridyl]pyrimidin-2-yl]amino]cyclohexanecarboxylic acid (277 mg, 670 μmol, 1.00 eq) in DMF (1.5 mL) was added DIEA (260 mg, 2.01 mmol, 350 μL, 3.00 eq) until pH-7. Then HATU (382 mg, 1.01 mmol, 1.50 eq) was added to the mixture of the two solutions. The mixture was stirred at 25 °C for 1 h. LC-MS showed the desired mass was detected. The reaction mixture was filtered to give a residue. The residue was purified by prep-HPLC (column: Waters Xbridge BEH C18 100*30 mm*10 um; mobile phase: [H₂O (10 mM NH₄HCO₃)-ACN]; gradient: 25%-55% B over 8.0 min) to give the title compound (Compound 546) (38.0 mg, 47.6 μmol, 7.10% yield, 100% purity) as a pale yellow solid. LCMS (ES⁺): m/z 798.4 [M+H]⁺.

### Example 554. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(6-(3-oxomorpholino)pyridin-2-yl)pyrimidin-2-yl)amino)cyclohexyl)cyclohexane-1-carboxamide (Compound 538)

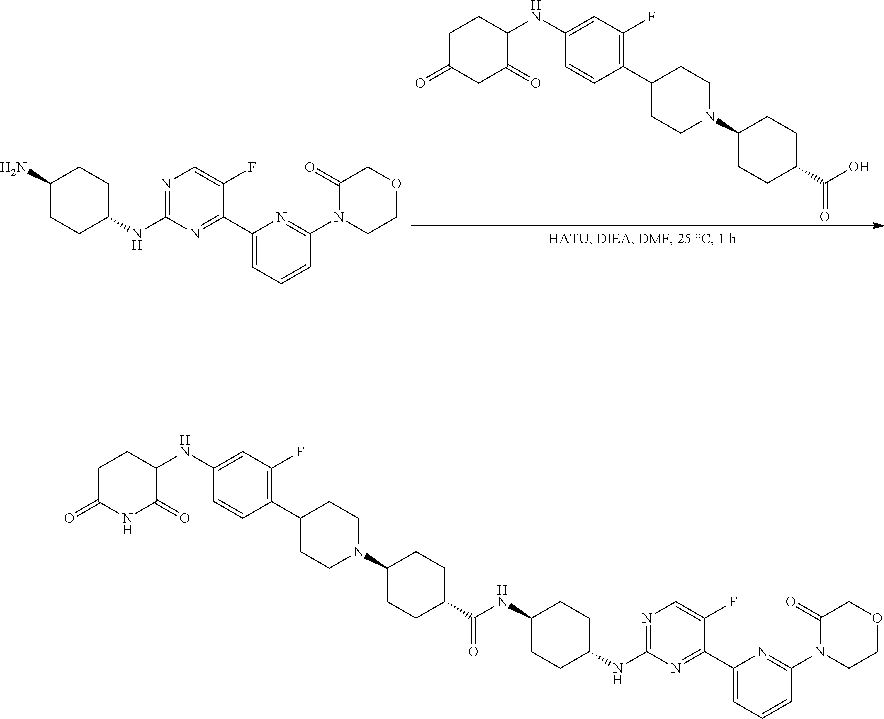 [SMILES: N[C@H]1CC[C@H](Nc2ncc(F)c(-c3cccc(N4CCOCC4=O)n3)n2)CC1 ; O=C1CCC(Nc2ccc(C3CCN([C@H]4CC[C@H](C(=O)O)CC4)CC3)c(F)c2)C(=O)C1 ; O=C1CCC(Nc2ccc(C3CCN([C@H]4CC[C@H](C(=O)N[C@H]5CC[C@H](Nc6ncc(F)c(-c7cccc(N8CCOCC8=O)n7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(6-(3-oxomorpholino)pyridin-2-yl)pyrimidin-2-yl)amino)cyclohexyl)cyclohexane-1-carboxamide (Compound 538). To a solution of trans-4-(4-(4-((2,4-dioxocyclohexyl)amino)-2-fluorophenyl)piperidin-1-yl)cyclohexane-1-carboxylic acid (265 mg, 614 μmol, 1.00 eq) in DMF (4 mL) were added HATU (81.7 mg, 215 μmol, 0.350 eq) and DIEA (663 mg, 3.08 mmol, 535 μL, 5.00 eq) at 25 °C, to the reaction was added trans-4-(6-(2-((4-aminocyclohexyl)amino)-5-fluoropyrimidin-4-yl)pyridin-2-yl)morpholin-3-one (123 mg, 246 μmol, 0.400 eq, TFA) at 25 °C, the mixture was stirred at 25 °C for 1 h. LCMS showed trans-4-(4-(4-((2,4-dioxocyclohexyl)amino)-2-fluorophenyl)piperidin-1-yl)cyclohexane-1-carboxylic acid was consumed completely and the desired mass was detected. The mixture was concentrated under reduced pressure to give a residue. The residue was purified by neutral prep-HPLC (column: Waters Xbridge Prep OBD C18 150*40 mm*10 um; mobile phase: [H₂O (10 mM NH₄HCO₃)-ACN]; gradient: 25%-55% B over 8.0 min) to give the title compound (Compound 538) (6.80 mg, 8.50 μmol, 1.38% yield, 100% purity) as a white solid. LCMS (ES⁺): m/z 800.4 [M+H]⁺

### Example 555. Synthesis of trans-N-(trans-4-(4-(4-((2,6-dioxopiperidin-3-yl)amino)-2-fluorophenyl)piperidin-1-yl)cyclohexyl)-4-((5-fluoro-4-(6-(3-oxomorpholino)pyridin-2-yl)pyrimidin-2-yl)amino)cyclohexane-1-carboxamide (Compound 545)

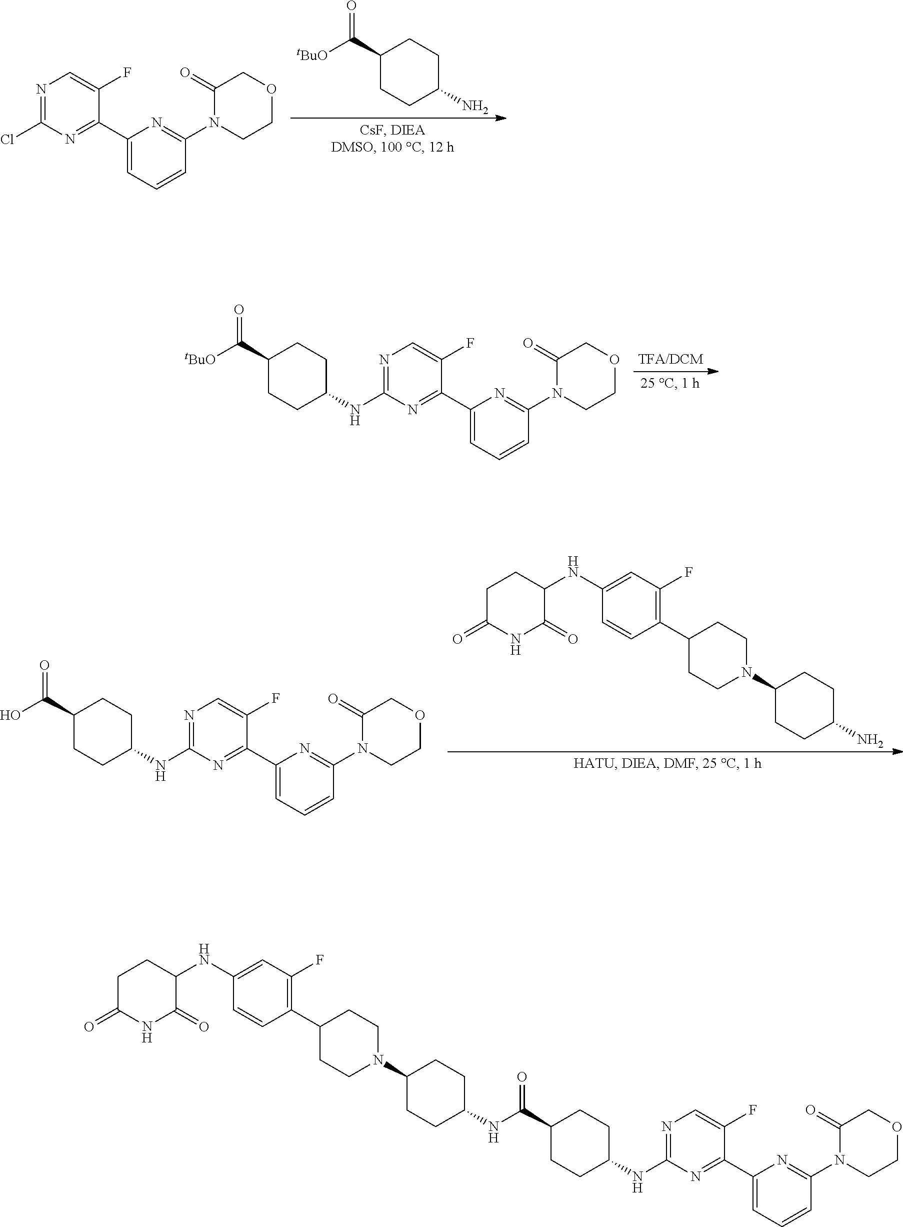 [SMILES: O=C1COCCN1c1cccc(-c2nc(Cl)ncc2F)n1 ; CC(C)(C)OC(=O)[C@H]1CC[C@H](N)CC1 ; CC(C)(C)OC(=O)[C@H]1CC[C@H](Nc2ncc(F)c(-c3cccc(N4CCOCC4=O)n3)n2)CC1 ; O=C1COCCN1c1cccc(-c2nc(N[C@H]3CC[C@H](C(=O)O)CC3)ncc2F)n1 ; N[C@H]1CC[C@H](N2CCC(c3ccc(NC4CCC(=O)NC4=O)cc3F)CC2)CC1 ; O=C1CCC(Nc2ccc(C3CCN([C@H]4CC[C@H](NC(=O)[C@H]5CC[C@H](Nc6ncc(F)c(-c7cccc(N8CCOCC8=O)n7)n6)CC5)CC4)CC3)c(F)c2)C(=O)N1]

Synthesis of tert-butyl trans-4-((5-fluoro-4-(6-(3-oxomorpholino)pyridin-2-yl)pyrimidin-2-yl)amino)cyclohexane-1-carboxylate. A mixture of 4-[6-(2-chloro-5-fluoro-pyrimidin-4-yl)-2-pyridyl]morpholin-3-one (2.00 g, 6.48 mmol, 1.00 eq), tert-butyl 4-aminocyclohexanecarboxylate (1.29 g, 6.48 mmol, 1 eq), DIEA (1.67 g, 12.9 mmol, 2.26 mL, 2.00 eq), CsF (1.97 g, 12.96 mmol, 478 μL, 2.00 eq) in DMSO (50 mL) at 20 °C was degassed and purged with N₂ for 3 times, and then the mixture was stirred at 100 °C for 12 h under N₂ atmosphere. LC-MS showed the desired mass was detected. The reaction mixture was quenched by addition H₂O 100 mL, and extracted with EtOAc 100 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (4 g, crude) as a yellow solid. LCMS (ES⁺): m/z 472.3 [M+H]⁺.

Synthesis of trans-4-((5-fluoro-4-(6-(3-oxomorpholino)pyridin-2-yl)pyrimidin-2-yl)amino)cyclohexane-1-carboxylic acid. To a solution of tert-butyl trans-4-((5-fluoro-4-(6-(3-oxomorpholino)pyridin-2-yl)pyrimidin-2-yl)amino)cyclohexane-1-carboxylate (500 mg, 1.06 mmol, 1.00 eq) in DCM (5 mL) was added TFA (3.07 g, 26.9 mmol, 2.00 mL, 25.3 eq) at 25 °C. The mixture was stirred at 25 °C for 1 h. LC-MS showed the desired mass was detected. The reaction mixture was concentrated under reduced pressure to give the title compound (700 mg, crude, TFA) as a yellow oil. LCMS (ES⁺): m/z 416.1 [M+H]⁺.

Synthesis of trans-N-(trans-4-(4-(4-((2,6-dioxopiperidin-3-yl)amino)-2-fluorophenyl)piperidin-1-yl)cyclohexyl)-4-((5-fluoro-4-(6-(3-oxomorpholino)pyridin-2-yl)pyrimidin-2-yl)amino)cyclohexane-1-carboxamide (Compound 545). To a solution of trans-4-((5-fluoro-4-(6-(3-oxomorpholino)pyridin-2-yl)pyrimidin-2-yl)amino)cyclohexane-1-carboxylic acid (400 mg, 755 μmol, 1.00 eq, TFA) and 3-[4-[1-(trans-4-aminocyclohexyl)-4-piperidyl]-3-fluoro-anilino]piperidine-2,6-dione (312 mg, 604 μmol, 0.800 eq, TFA) in DMF (5 mL) was added DIEA (195 mg, 1.51 mmol, 263 μL, 2.00 eq) and HATU (574 mg, 1.51 mmol, 2.00 eq) at 25 °C. The mixture was stirred at 25 °C for 1 h. LC-MS showed the desired mass was detected. The reaction mixture was concentrated under reduced pressure. The residue was purified by prep-HPLC (column: 3_Phenomenex Luna C18 75*30 mm*3 um; mobile phase: [H₂O (0.2% FA)-ACN]; gradient: 5%-35% B over 8.0 min) to give the title compound (Compound 545) (80.2 mg, 94.9 μmol, 12.5% yield, 94.6% purity) as a pale yellow solid. LCMS (ES⁺): m/z 800.4 [M+H]⁺.

### Example 556. Synthesis of trans-N-((trans)-4-(4-(4-((2,6-dioxopiperidin-3-yl)amino)-2-fluorophenyl)piperidin-1-yl)cyclohexyl)-4-((5-fluoro-4-(6-(1-hydroxycyclobutyl)pyridin-2-yl)pyrimidin-2-yl)amino)cyclohexane-1-carboxamide (Compound 479)

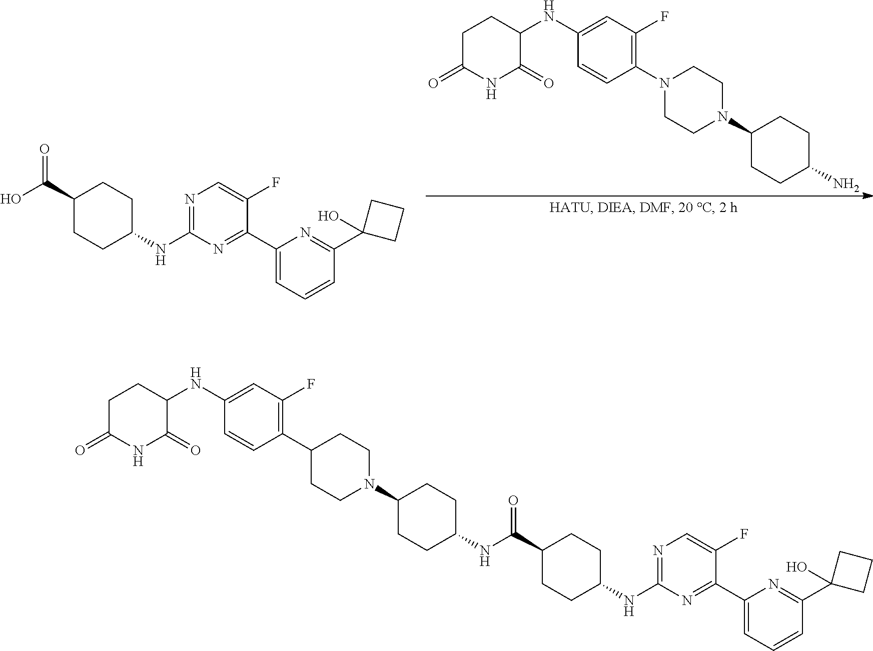 [SMILES: O=C(O)[C@H]1CC[C@H](Nc2ncc(F)c(-c3cccc(C4(O)CCC4)n3)n2)CC1 ; N[C@H]1CC[C@H](N2CCN(c3ccc(NC4CCC(=O)NC4=O)cc3F)CC2)CC1 ; O=C1CCC(Nc2ccc(C3CCN([C@H]4CC[C@H](NC(=O)[C@H]5CC[C@H](Nc6ncc(F)c(-c7cccc(C8(O)CCC8)n7)n6)CC5)CC4)CC3)c(F)c2)C(=O)N1]

Synthesis of trans-N-((trans)-4-(4-(4-((2,6-dioxopiperidin-3-yl)amino)-2-fluorophenyl)piperidin-1-yl)cyclohexyl)-4-((5-fluoro-4-(6-(I-hydroxycyclobutyl)pyridin-2-yl)pyrimidin-2-yl)amino)cyclohexane-1-carboxamide (Compound 479). To a solution of trans-4-((5-fluoro-4-(6-(1-hydroxycyclobutyl)pyridin-2-yl)pyrimidin-2-yl)amino)cyclohexane-1-carboxylic acid (40.0 mg, 103 μmol, 1.00 eq) in DMF (1 mL) was added HATU (39.3 mg, 103 μmol, 1.00 eq), trans-3-((4-(1-(4-aminocyclohexyl)piperidin-4-yl)-3-fluorophenyl)amino)piperidine-2,6-dione (41.6 mg, 103 μmol, 1.00 eq) and DIEA (26.7 mg, 207 μmol, 36.0 μL, 2.00 eq) at 25 °C. The mixture was stirred at 25 °C for 2 h. LC-MS showed trans-4-((5-fluoro-4-(6-(1-hydroxycyclobutyl)pyridin-2-yl)pyrimidin-2-yl)amino)cyclohexane-1-carboxylic acid was consumed completely and the desired mass was detected. The reaction mixture was concentrated under reduced pressure. The residue was purified by prep-HPLC (column: Phenomenex Luna C18 75*30 mm*3 um; mobile phase: [H₂O (0.2% FA)-ACN]; gradient: 5%-45% B over 8.0 min) to give the title compound (Compound 479) (27.6 mg, 35.3 μmol, 34.1% yield, 98.8% purity) as a pale yellow solid. LCMS (ES⁺): m/z 771.5 [M+H]⁺.

### Example 557. Synthesis of trans-4-(4-(4-((2,6-dioxopiperidin-3-yl)amino)-2-fluorophenyl)piperidin-1-yl)-N-(trans-4-((5-fluoro-4-(3-((S)-3-hydroxypyrrolidine-1-carbonyl)phenyl)pyrimidin-2-yl)amino)cyclohexyl)cyclohexane-1-carboxamide (Compound 511)

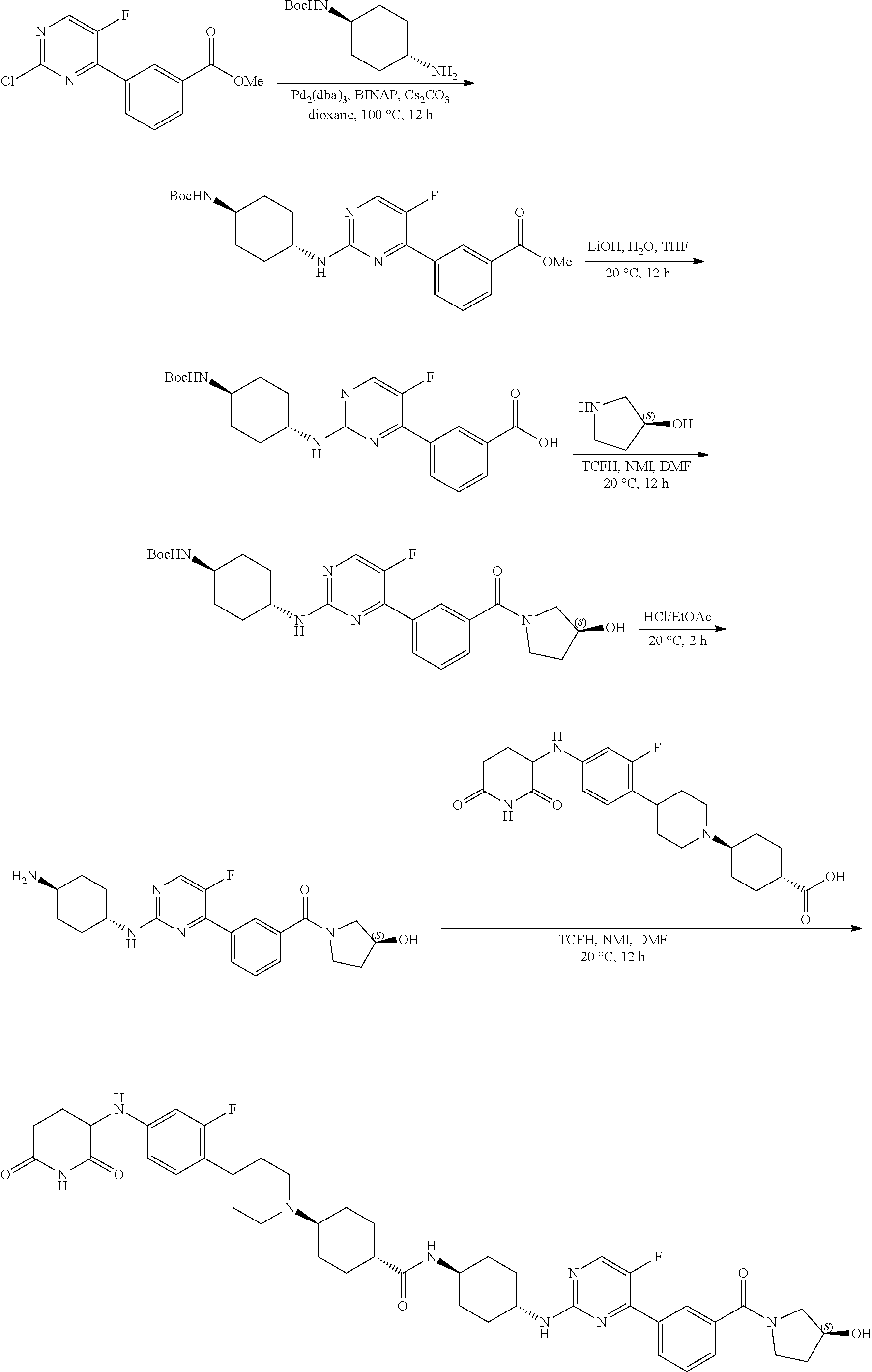

Synthesis of trans-methyl 3-(2-(((1r,4r)-4-((tert-butoxycarbonyl)amino)cyclohexyl)amino)-5-fluoropyrimidin-4-yl)benzoate. To a solution of methyl 3-(2-chloro-5-fluoropyrimidin-4-yl)benzoate (4.00 g, 15.0 mmol, 1.00 eq) and trans-tert-butyl ((1r,4r)-4-aminocyclohexyl)carbamate (3.21 g, 15.0 mmol, 1.00 eq) in dioxane (60 mL) was added Pd₂(dba)₃ (1.37 g, 1.50 mmol, 0.100 eq), Cs₂CO₃ (9.77 g, 30.0 mmol, 2.00 eq) and BINAP (1.87 g, 3.00 mmol, 0.200 eq) at 20 °C. The mixture was stirred at 100 °C for 12 h under N₂. LC-MS showed that methyl 3-(2-chloro-5-fluoropyrimidin-4-yl)benzoate was consumed completely and one main peak with desired MS was detected. The reaction mixture was filtered and the filtrate was diluted with H₂O (100 mL) and extracted with EtOAc (100 mL*3). The combined organic layers were washed with brine (100 mL), dried over anhydrous Na₂SO₄, filtered and concentrated under reduced pressure to give a residue. The crude product was triturated with PE (200 mL) and EtOAc (20 mL) at 20 °C for 30 min to give title compound (5.50 g, crude) as a yellow solid. LCMS (ES⁺): m/z 445.2 [M+H]⁺

Synthesis of trans-3-(2-(((1r,4r)-4-((tert-butoxycarbonyl)amino)cyclohexyl)amino)-5-fluoropyrimidin-4-yl)benzoic acid. To a solution of trans-methyl 3-(2-(((1r,4r)-4-((tert-butoxycarbonyl)amino)cyclohexyl)amino)-5-fluoropyrimidin-4-yl)benzoate (4.50 g, 10.1 mmol, 1.00 eq) in THF (50 mL) and H₂O (10 mL) was added LiOH (727 mg, 30.4 mmol, 3.00 eq) at 20 °C. The mixture was stirred at 20 °C for 12 h. LCMS showed that trans-methyl 3-(2-(((1r,4r)-4-((tert-butoxycarbonyl)amino)cyclohexyl)amino)-5-fluoropyrimidin-4-yl)benzoate was consumed completely and one main peak with desired MS was detected. The reaction mixture was concentrated under reduced pressure to remove solvent. The residue was diluted with H₂O (30 mL) and extracted with EtOAc (10 mL*3). The water layer was adjusted pH to 4-5 with 1 M HCl, filtered and the filter cake was dried under reduced pressure to give title compound (2.90 g, crude) as a yellow solid. LCMS (ES⁺): m/z 431.0 [M+H]⁺

Synthesis of trans-tert-butyl ((4-((5-fluoro-4-(3-((S)-3-hydroxypyrrolidine-1-carbonyl)phenyl)pyrimidin-2-yl)amino)cyclohexyl)carbamate. To a solution of trans-3-(2-(((1r,4r)-4-((tert-butoxycarbonyl)amino)cyclohexyl)amino)-5-fluoropyrimidin-4-yl)benzoic acid (3.00 g, 6.97 mmol, 1.00 eq) in DMF (30 mL) was added TCFH (4.30 g, 15.3 mmol, 2.20 eq), NMI (3.43 g, 41.8 mmol, 3.33 mL, 6.00 eq) and (S)-pyrrolidin-3-ol (607 mg, 6.97 mmol, 563 μL, 1.00 eq) at 20 °C. The mixture was stirred at 20 °C for 12 h. LCMS showed that trans-3-(2-(((1r,4r)-4-((tert-butoxycarbonyl)amino)cyclohexyl)amino)-5-fluoropyrimidin-4-yl)benzoic acid was consumed completely and one main peak with desired MS was detected. The reaction mixture was diluted with H₂O (50 mL) and extracted with EtOAc (30 mL*3). The combined organic layers were washed with brine (50 mL), dried over anhydrous Na₂SO₄, filtered and concentrated under reduced pressure to give a residue. The residue was purified by column chromatography (SiO₂, Petroleum ether/Ethyl acetate=100/1 to 10/90) to give title compound (2.90 g, 5.80 mmol, 83.3% yield, N/A purity) as a yellow solid. LCMS (ES+): m/z 500.2 [M+H]⁺

Synthesis of trans-(3-(2-((4-aminocyclohexyl)amino)-5-fluoropyrimidin-4-yl)phenyl)((S)-3-hydroxypyrrolidin-1-yl)methanone. A mixture of trans-tert-butyl (4-((5-fluoro-4-(3-((S)-3-hydroxypyrrolidine-1-carbonyl)phenyl)pyrimidin-2-yl)amino)cyclohexyl)carbamate (350 mg, 701 μmol, 1.00 eq) in HCl/EtOAc (4 M, 3.50 mL, 20.0 eq) was stirred at 20 °C for 2 h. LCMS showed that trans-tert-butyl ((4-((5-fluoro-4-(3-((S)-3-hydroxypyrrolidine-1-carbonyl)phenyl)pyrimidin-2-yl)amino)cyclohexyl)carbamate was consumed completely and one main peak with desired MS was detected. The reaction mixture was concentrated under reduced pressure to give title compound (300 mg, crude, HCl) as a white solid. LCMS (ES⁺): m/z 400.2 [M+H]⁺

Synthesis of trans-4-(4-(4-((2,6-dioxopiperidin-3-yl)amino)-2-fluorophenyl)piperidin-1-yl)-N-(trans-4-((5-fluoro-4-(3-((S)-3-hydroxypyrrolidine-1-carbonyl)phenyl)pyrimidin-2-yl)amino)cyclohexyl)cyclohexane-1-carboxamide (Compound 511). To a solution of trans-(3-(2-((4-aminocyclohexyl)amino)-5-fluoropyrimidin-4-yl)phenyl)((S)-3-hydroxypyrrolidin-1-yl)methanone (300 mg, 688 μmol, 1.00 eq, HCl) and trans-4-(4-(4-((2,6-dioxopiperidin-3-yl)amino)-2-fluorophenyl)piperidin-1-yl)cyclohexane-1-carboxylic acid (300 mg, 550 μmol, 7.99e-1 eq, TFA) in DMF (3 mL) was added TCFH (386 mg, 1.38 mmol, 2.00 eq) and NMI (282 mg, 3.44 mmol, 274 μL, 5.00 eq) at 20 °C. The mixture was stirred at 20 °C for 12 h. LCMS showed that trans-(3-(2-((4-aminocyclohexyl)amino)-5-fluoropyrimidin-4-yl)phenyl)((S)-3-hydroxypyrrolidin-1-yl)methanone was consumed completely and one main peak with desired MS was detected. The reaction mixture was concentrated under reduced pressure to remove solvent. The residue was purified by prep-HPLC (column: Phenomenex luna C18 100*40 mm*5 um; mobile phase: [H₂O (0.2% FA)-ACN]; gradient: 10%-50% B over 8.0 min) to give title compound (Compound 511) (97.2 mg, 106 μmol, 15.5% yield, 92.1% purity, 0.63 FA) as a white solid. LCMS (ES⁺): m/z 813.3 [M+H]⁺

### Example 558. Synthesis of trans-4-(4-(4-((2,6-dioxopiperidin-3-yl)amino)-2-fluorophenyl)piperazin-1-yl)-N-(trans-4-((5-fluoro-4-(3-((S)-3-hydroxypyrrolidine-1-carbonyl)phenyl)pyrimidin-2-yl)amino)cyclohexyl)cyclohexane-1-carboxamide (Compound 537)

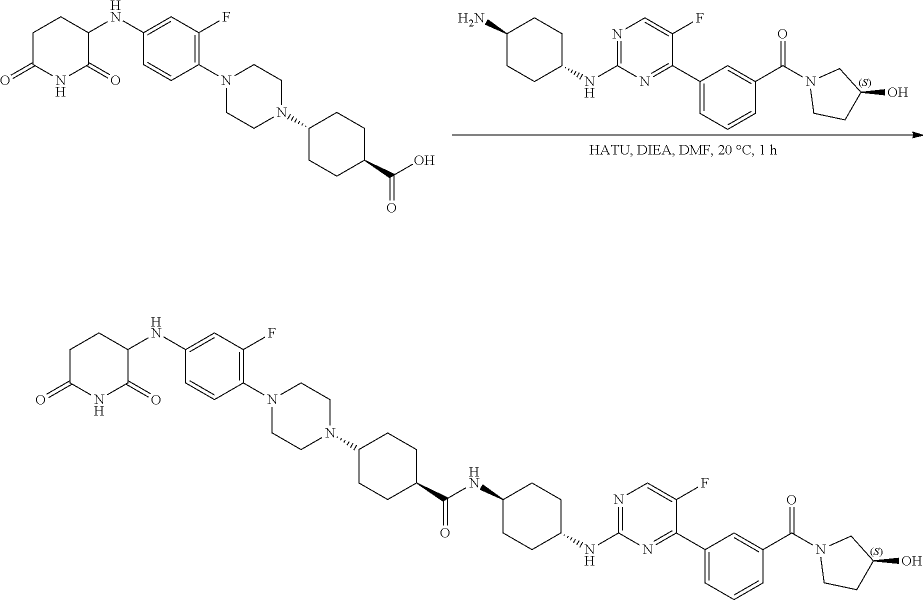

Synthesis of trans-4-(4-(4-((2,6-dioxopiperidin-3-yl)amino)-2-fluorophenyl)piperazin-1-yl)-N-(trans-4-((5-fluoro-4-(3-((S)-3-hydroxypyrrolidine-1-carbonyl)phenyl)pyrimidin-2-yl)amino)cyclohexyl)cyclohexane-1-carboxamide (Compound 537) To a solution of trans-4-(4-(4-((2,6-dioxopiperidin-3-yl)amino)-2-fluorophenyl)piperazin-1-yl)cyclohexane-1-carboxylic acid (350 mg, 640 μmol, 1.00 eq, TFA) in DMF (5 mL) were added HATU (487 mg, 1.28 mmol, 2.00 eq), DIEA (414 mg, 3.20 mmol, 558 μL, 5.00 eq) and trans-(3-(2-((4-aminocyclohexyl)amino)-5-fluoropyrimidin-4-yl)phenyl)((S)-3-hydroxypyrrolidin-1-yl)methanone (206 mg, 4726 μmol, 7.37e-1 eq, HCl) at 20 °C. The mixture was stirred at 20 °C for 1 h. LC-MS showed that trans-4-(4-(4-((2,6-dioxopiperidin-3-yl)amino)-2-fluorophenyl)piperazin-1-yl)cyclohexane-1-carboxylic acid was consumed completely and one main peak with desired MS was detected. The reaction mixture was concentrated under reduced pressure to remove solvent. The reaction mixture was purified by prep-HPLC (column: Waters Xbridge Prep OBD C18 150*40 mm*10 um; mobile phase: [H₂O (10 mM NH₄HCO₃)-ACN]; gradient: 20%-55% B over 8.0 min) to give title compound (Compound 537) (153 mg, 173 μmol, 26.9% yield, 91.7% purity) as a pale yellow solid. LCMS (ES⁺): m/z 814.4 [M+H]⁺.

### Example 559. Synthesis of trans-N-(trans-4-(4-(4-((2,6-dioxopiperidin-3-yl)amino)-2-fluorophenyl)piperidin-1-yl)cyclohexyl)-4-((5-fluoro-4-(3-((S)-3-hydroxypyrrolidine-1-carbonyl)phenyl)pyrimidin-2-yl)amino)cyclohexane-1-carboxamide (Compound 510)

Synthesis of trans-N-(trans-4-(4-(4-((2,6-dioxopiperidin-3-yl)amino)-2-fluorophenyl)piperidin-1-yl)cyclohexyl)-4-((5-fluoro-4-(3-((S)-3-hydroxypyrrolidine-1-carbonyl)phenyl)pyrimidin-2-yl)amino)cyclohexane-1-carboxamide (Compound 510). To a solution of trans-4-((5-fluoro-4-(3-((S)-3-hydroxypyrrolidine-1-carbonyl)phenyl)pyrimidin-2-yl)amino)cyclohexane-1-carboxylic acid (250 mg, 538 μmol, 1.00 eq, HCl) in DMF (3 mL) was added HATU (409 mg, 1.08 mmol, 2.00 eq), DIEA (347 mg, 2.69 mmol, 468 μL, 5.00 eq) and trans-3-((4-(1-(4-aminocyclohexyl)piperidin-4-yl)-3-fluorophenyl)amino)piperidine-2,6-dione (278 mg, 538 μmol, 1.00 eq, TFA) at 20 °C. The mixture was stirred at 20 °C for 12 h. LCMS showed that trams-4-((5-fluoro-4-(3-((S)-3-hydroxypyrrolidine-1-carbonyl)phenyl)pyrimidin-2-yl)amino)cyclohexane-1-carboxylic acid was consumed completely and one main peak with desired MS was detected. The reaction mixture was concentrated under reduced pressure to remove solvent. The residue was purified by prep-HPLC (column: Phenomenex luna C18 100*40 mm*3 um; mobile phase: [H₂O (0.2% FA)-ACN]; gradient: 5%-35% B over 8.0 min). The fraction was concentrated under reduced pressure. The residue was purified by prep-HPLC (column: Phenomenex luna C18 100*40 mm*5 um; mobile phase: [H₂O (0.2% FA)-ACN]; gradient: 10%-50% B over 8.0 min) to give title compound (Compound 510) (9.50 mg, 11.1 μmol, 2.06% yield, 97.1% purity, 0.43 FA) as a yellow solid. LCMS (ES⁺): m/z 813.3 [M+H]⁺

### Example 560. Synthesis of trans-N-(trans-4-(4-(4-((2,6-dioxopiperidin-3-yl)amino)-2-fluorophenyl)piperazin-1-yl)cyclohexyl)-4-((5-fluoro-4-(3-((S)-3-hydroxypyrrolidine-1-carbonyl)phenyl)pyrimidin-2-yl)amino)cyclohexane-1-carboxamide (Compound 509)

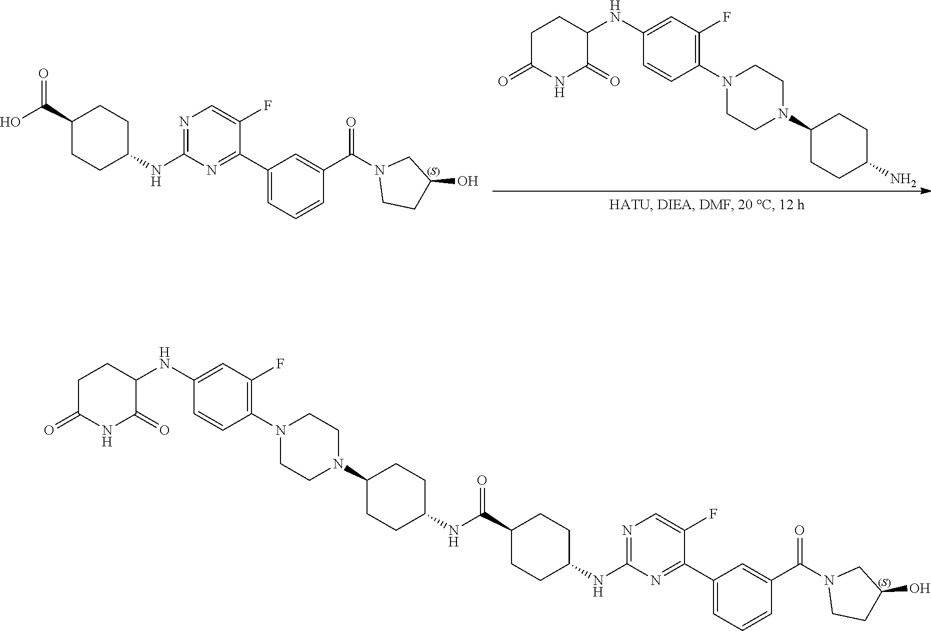

Synthesis of trans-N-(trans-4-(4-(4-((2,6-dioxopiperidin-3-yl)amino)-2-fluorophenyl)piperazin-1-yl)cyclohexyl)-4-((5-fluoro-4-(3-((S)-3-hydroxypyrrolidine-1-carbonyl)phenyl)pyrimidin-2-yl)amino)cyclohexane-1-carboxamide (Compound 509). To a solution of trans-4-((5-fluoro-4-(3-((S)-3-hydroxypyrrolidine-1-carbonyl)phenyl)pyrimidin-2-yl)amino)cyclohexane-1-carboxylic acid (250 mg, 583 μmol, 1.00 eq) in DMF (5 mL) was added HATU (444 mg, 1.17 mmol, 2.00 eq), DIEA (377 mg, 2.92 mmol, 508 μL, 5.00 eq) and trans-3-((4-(4-(4-aminocyclohexyl)piperazin-1-yl)-3-fluorophenyl)amino)piperidine-2,6-dione (300 mg, 580 μmol, 9.93e-1 eq, TFA) at 20 °C. The mixture was stirred at 20 °C for 12 h. LCMS showed that trans-4-((5-fluoro-4-(3-((S)-3-hydroxypyrrolidine-1-carbonyl)phenyl)pyrimidin-2-yl)amino)cyclohexane-1-carboxylic acid was consumed completely and one main peak with desired MS was detected. The reaction mixture was concentrated under reduced pressure to remove solvent. The residue was purified by prep-HPLC (column: Phenomenex luna C18 100*40 mm*5 um; mobile phase: [H₂O (0.2% FA)-ACN]; gradient: 5%-40% B over 8.0 min) to give title compound (Compound 509) (33.1 mg, 38.3 μmol, 6.56% yield, 94.1% purity) as a pale yellow solid. LCMS (ES⁺): m/z 814.5 [M+H]⁺

### Example 561. Synthesis of trans-3-((3-fluoro-4-(4-((1-(4-((5-fluoro-4-(3-((S)-3-hydroxypyrrolidine-1-carbonyl)phenyl)pyrimidin-2-yl)amino)cyclohexane-1-carbonyl)piperidin-4-yl)methyl)piperazin-1-yl)phenyl)amino)piperidine-2,6-dione (Compound 483)

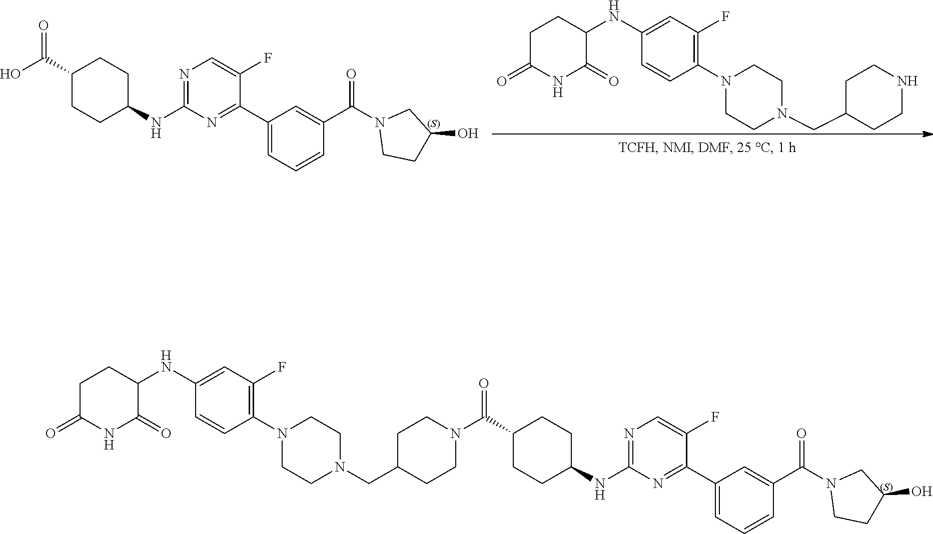

Synthesis of trans-3-((3-fluoro-4-(4-((1-(4-((5-fluoro-4-(3-((S)-3-hydroxypyrrolidine-1-carbonyl)phenyl)pyrimidin-2-yl)amino)cyclohexane-1-carbonyl)piperidin-4-yl)methyl)piperazin-1-yl)phenyl)amino)piperidine-2,6-dione (Compound 483). To a solution of trans-(S)-4-((5-fluoro-4-(3-(3-hydroxypyrrolidine-1-carbonyl)phenyl)pyrimidin-2-yl)amino)cyclohexane-1-carboxylic acid (66.2 mg, 155 μmol, 0.400 eq) in DMF (5 mL) were added TCFH (239 mg, 850 μmol, 2.20 eq) and NMI (63.5 mg, 773 μmol, 61.6 μL, 2.00 eq) at 25 °C. Then 3-[3-fluoro-4-[4-(4-piperidylmethyl)piperazin-1-yl]anilino]piperidine-2, 6-dione (200 mg, 386 μmol, 1.00 eq, TFA) was added to the reaction mixture at 25 °C. The mixture was stirred at 25 °C for 1 h. LCMS showed trans-(S)-4-((5-fluoro-4-(3-(3-hydroxypyrrolidine-1-carbonyl)phenyl)pyrimidin-2-yl)amino)cyclohexane-1-carboxylic acid was consumed completely and the desired mass was detected. The mixture was filtered and the filtrate was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 25%-55% B over 8.0 min) to give the title compound (Compound 483) (10.0 mg, 11.3 μmol, 2.92% yield, 91.9% purity) as a pale yellow solid. LCMS (ES⁺): m/z 814.4 [M+H]⁺.

### Example 562. Synthesis of trans-3-((3-fluoro-4-(4-((1-(4-((5-fluoro-4-(3-((S)-3-hydroxypyrrolidine-1-carbonyl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 482)

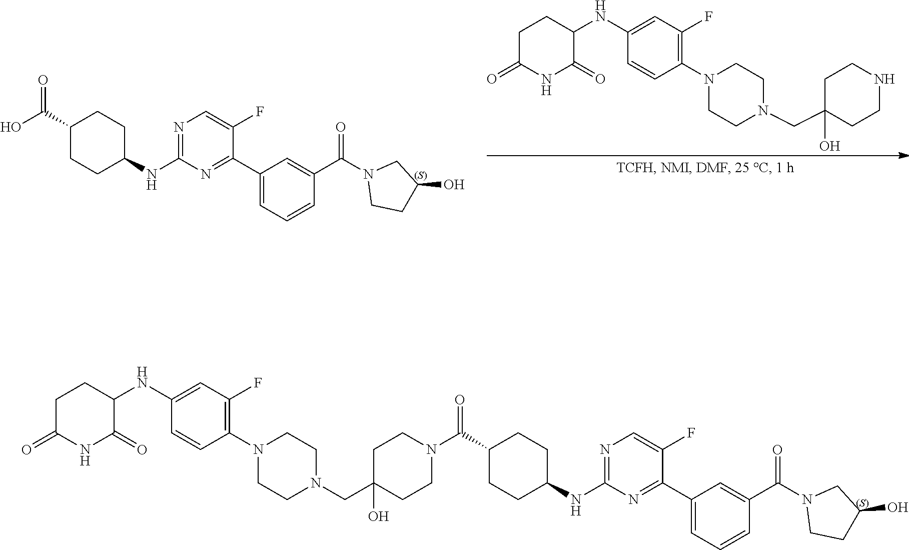

Synthesis of trans-3-((3-fluoro-4-(4-((1-(4-((5-fluoro-4-(3-((S)-3-hydroxypyrrolidine-1-carbonyl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 482). To a solution of trans-(S)-4-((5-fluoro-4-(3-(3-hydroxypyrrolidine-1-carbonyl)phenyl)pyrimidin-2-yl)amino)cyclohexane-1-carboxylic acid (66.0 mg, 154 μmol, 0.400 eq) in DMF (5 mL) were added TCFH (357 mg, 1.27 mmol, 3.30 eq), NMI (94.8 mg, 1.92 mmol, 153 μL, 5.00 eq) and 3-((3-fluoro-4-(4-((4-hydroxypiperidin-4-yl)methyl)piperazin-1-yl)phenyl)amino)piperidine-2,6-dione (205 mg, 385 μmol, 1.00 eq, TFA) at 25 °C. The mixture was stirred at 25 °C for 1 h. LCMS showed trans-(S)-4-((5-fluoro-4-(3-(3-hydroxypyrrolidine-1-carbonyl)phenyl)pyrimidin-2-yl)amino)cyclohexane-1-carboxylic acid was consumed completely and the desired mass was detected. The reaction mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 18%-48% B over 8.0 min) to give the title compound (Compound 482) (9.00 mg, 10.0 μmol, 2.60% yield, 92.2% purity) as a pale yellow solid. LCMS (ES⁺): m/z 830.4 [M+H]⁺.

### Example 563. Synthesis of trans-3-((3-fluoro-4-(1-((1-(4-((5-fluoro-4-(3-((S)-3-hydroxypyrrolidine-1-carbonyl)phenyl)pyrimidin-2-yl)amino)cyclohexane-1-carbonyl)piperidin-4-yl)methyl)piperidin-4-yl)phenyl)amino)piperidine-2,6-dione (Compound 468)

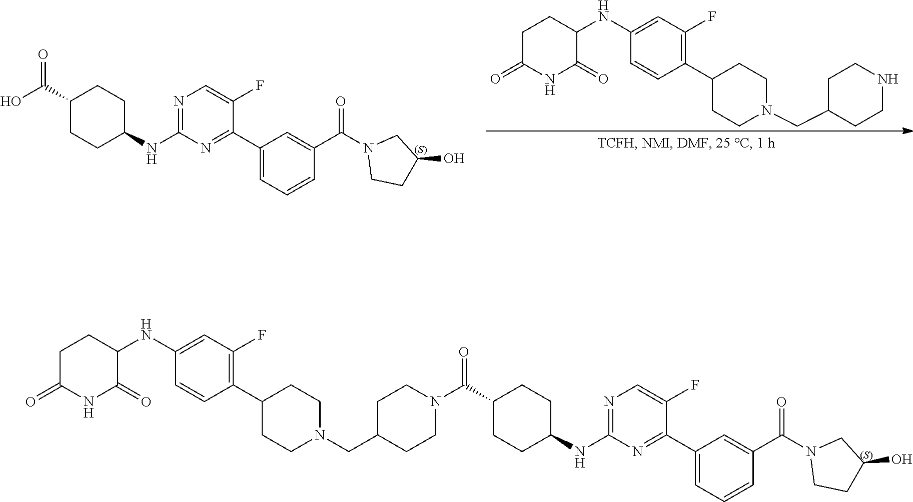

Synthesis of trans-3-((3-fluoro-4-(1-((1-(4-((5-fluoro-4-(3-((S)-3-hydroxypyrrolidine-1-carbonyl)phenyl)pyrimidin-2-yl)amino)cyclohexane-1-carbonyl)piperidin-4-yl)methyl)piperidin-4-yl)phenyl)amino)piperidine-2,6-dione (Compound 468). To a solution of trans-4-((5-fluoro-4-(3-((S)-3-hydroxypyrrolidine-1-carbonyl)phenyl)pyrimidin-2-yl)amino)cyclohexane-1-carboxylic acid (83.0 mg, 194 μmol, 0.500 eq) in DMF (5 mL) were added TCFH (239 mg, 852 μmol, 2.20 eq), NMI (159 mg, 1.94 mmol, 154 μL, 5.00 eq) and 3-[3-fluoro-4-[1-(4-piperidylmethyl)-4-piperidyl]anilino]piperidine-2,6-dione (200 mg, 387 μmol, 1.00 eq, TFA) at 25 °C. The mixture was stirred at 25 °C for 1 h. LCMS showed trans-4-((5-fluoro-4-(3-((S)-3-hydroxypyrrolidine-1-carbonyl)phenyl)pyrimidin-2-yl)amino)cyclohexane-1-carboxylic acid was consumed completely and the desired mass was detected. The reaction mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 10%-40% B over 8.0 min) to give the title compound 468) (13.1 mg, 15.3 μmol, 3.94% yield, 100% purity, FA) as a white solid. LCMS (ES⁺): m/z 813.4 [M+H]⁺.

### Example 564. Synthesis of trans-3-((3-fluoro-4-(1-((1-(4-((5-fluoro-4-(3-((S)-3-hydroxypyrrolidine-1-carbonyl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 481)

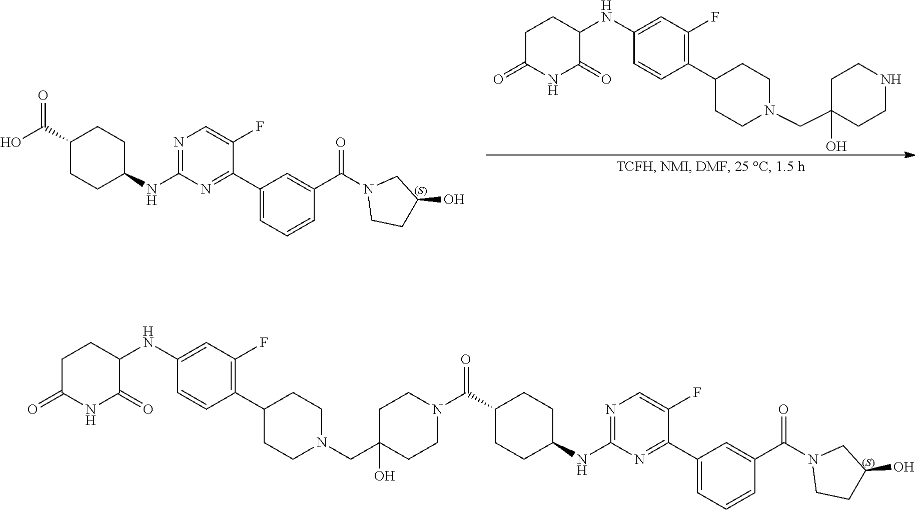

Synthesis of trans-3-((3-fluoro-4-(1-((1-(4-((5-fluoro-4-(3-((S)-3-hydroxypyrrolidine-1-carbonyl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 481). To a solution of trans-(S)-4-((5-fluoro-4-(3-(3-hydroxypyrrolidine-1-carbonyl)phenyl)pyrimidin-2-yl)amino)cyclohexane-1-carboxylic acid (74.2 mg, 173 μmol, 0.450 eq) in DMF (5 mL) were added TCFH (324 mg, 1.16 mmol, 3.00 eq), NMI (158 mg, 1.92 mmol, 154 μL, 5.00 eq) and 3-((3-fluoro-4-(1-((4-hydroxypiperidin-4-yl)methyl)piperidin-4-yl)phenyl)amino)piperidine-2,6-dione (205 mg, 385 μmol, 1.00 eq, TFA) at 25 °C. The mixture was stirred at 25 °C for 1.5 h. LC-MS showed trans-(S)-4-((5-fluoro-4-(3-(3-hydroxypyrrolidine-1-carbonyl)phenyl)pyrimidin-2-yl)amino)cyclohexane-1-carboxylic acid was consumed completely and the desired mass was detected. The reaction mixture was concentrated under reduced pressure to remove solvent to give the title compound (Compound 481) (28.7 mg, 33.0 μmol, 8.57% yield, 95.3% purity) as a white solid. LCMS (ES⁺): m/z 829.5 [M+H]⁺

### Example 565. Synthesis of trans-N-(trans-4-((4-(3-cyclopropylphenyl)-5-fluoropyrimidin-2-yl)amino)cyclohexyl)-4-(4-(4-((2,6-dioxopiperidin-3-yl)amino)-2-fluorophenyl)piperazin-1-yl)cyclohexane-1-carboxamide (Compound 559)

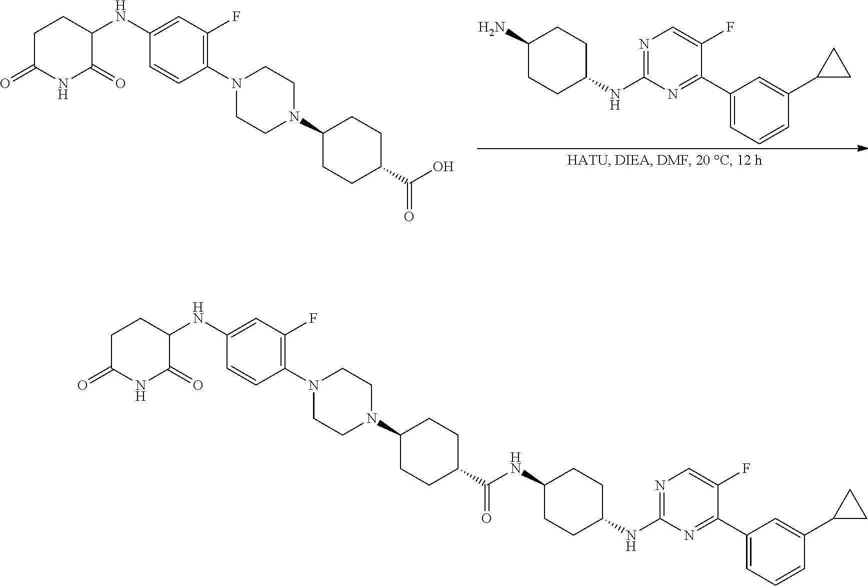 [SMILES: O=C1CCC(Nc2ccc(N3CCN([C@H]4CC[C@H](C(=O)O)CC4)CC3)c(F)c2)C(=O)N1 ; N[C@H]1CC[C@H](Nc2ncc(F)c(-c3cccc(C4CC4)c3)n2)CC1 ; O=C1CCC(Nc2ccc(N3CCN([C@H]4CC[C@H](C(=O)N[C@H]5CC[C@H](Nc6ncc(F)c(-c7cccc(C8CC8)c7)n6)CC5)CC4)CC3)c(F)c2)C(=O)N1]

Synthesis of trans-N-(trans-4-((4-(3-cyclopropylphenyl)-5-fluoropyrimidin-2-yl)amino)cyclohexyl)-4-(4-(4-((2,6-dioxopiperidin-3-yl)amino)-2-fluorophenyl)piperazin-1-yl)cyclohexane-1-carboxamide (Compound 559). To a solution of trans-4-(4-(4-((2,6-dioxopiperidin-3-yl)amino)-2-fluorophenyl)piperazin-1-yl)cyclohexane-1-carboxylic acid (400 mg, 732 μmol, 1.00 eq, TFA) in DMF (5 mL) were added HATU (557 mg, 1.46 mmol, 2.00 eq) and DIEA (473 mg, 3.66 mmol, 637 μL, 5.00 eq) and trans-(1r,4r)-N1-(4-(3-cyclopropylphenyl)-5-fluoropyrimidin-2-yl)cyclohexane-1,4-diamine (237 mg, 539 μmol, 7.36e-1 eq, TFA) at 20 °C. The mixture was stirred at 20 °C for 12 h. LCMS showed that trans-4-(4-(4-((2,6-dioxopiperidin-3-yl)amino)-2-fluorophenyl)piperazin-1-yl)cyclohexane-1-carboxylic acid was consumed completely and one main peak with desired MS was detected. The reaction mixture was concentrated under reduced pressure to remove solvent. The reaction mixture was purified by prep-HPLC (column: Phenomenex luna C18 100*40 mm*5 um; mobile phase: [H₂O (0.2% FA)-ACN]; gradient: 20%-60% B over 8.0 min) to give title compound (Compound 559) (166 mg, 221 μmol, 30.2% yield, 98.7% purity) as a yellow solid. LCMS (ES⁺): m/z 741.4 [M+H]⁺.

### Example 566. Synthesis of trans-N-(4-((4-(3-cyclopropylphenyl)-5-fluoropyrimidin-2-yl)amino)cyclohexyl)-1′-(4-((2,6-dioxopiperidin-3-yl)amino)-2-fluorophenyl)-[1,4′-bipiperidine]-4-carboxamide (Compound 504)

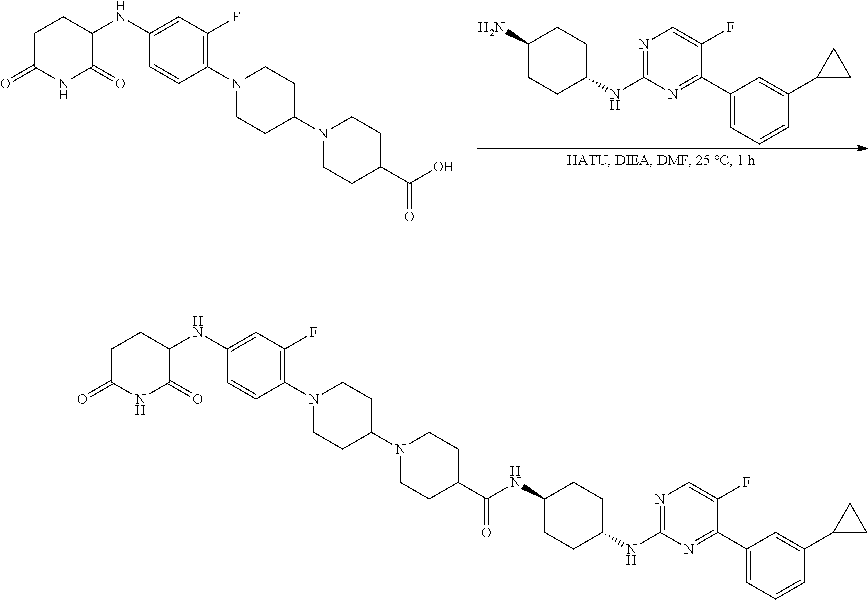 [SMILES: O=C1CCC(Nc2ccc(N3CCC(N4CCC(C(=O)O)CC4)CC3)c(F)c2)C(=O)N1 ; N[C@H]1CC[C@H](Nc2ncc(F)c(-c3cccc(C4CC4)c3)n2)CC1 ; O=C1CCC(Nc2ccc(N3CCC(N4CCC(C(=O)N[C@H]5CC[C@H](Nc6ncc(F)c(-c7cccc(C8CC8)c7)n6)CC5)CC4)CC3)c(F)c2)C(=O)N1]

Synthesis trans-N-(4-((4-(3-cyclopropylphenyl)-5-fluoropyrimidin-2-yl)amino)cyclohexyl)-1′-(4-((2,6-dioxopiperidin-3-yl)amino)-2-fluorophenyl)-[1,4′-bipiperidine]-4-carboxamide (Compound 504). To a solution of 1′-(4-((2,6-dioxopiperidin-3-yl)amino)-2-fluorophenyl)-[1,4′-bipiperidine]-4-carboxylic acid (300 mg, 549 μmol, 1.00 eq, TFA) in DMF (5 mL) was added trans-(1r,4r)-N1-(4-(3-cyclopropylphenyl)-5-fluoropyrimidin-2-yl)cyclohexane-1,4-diamine (89.6 mg, 203 μmol, 0.37 eq, TFA), DIEA (213 mg, 1.65 mmol, 287 μL, 3.00 eq) and HATU (313 mg, 823 μmol, 1.50 eq) at 25 °C. The mixture was stirred at 25 °C for 1 h. LC-MS showed 1′-(4-((2,6-dioxopiperidin-3-yl)amino)-2-fluorophenyl)-[1,4′-bipiperidine]-4-carboxylic acid was consumed completely and the desired mass was detected. The reaction mixture was concentrated under reduced pressure to give a residue. The residue was purified by prep-HPLC (column: Phenomenex Luna C18 100*30 mm*3 um; mobile phase: [H₂O (0.04% HCl)-ACN]; gradient: 15%-45% B over 8.0 min) to give the title Compound (Compound 504) as a pale yellow solid. LCMS (ES⁺): m/z 741.4 [M+H]⁺.

### Example 567. Synthesis of trans-N-((trans)-4-((4-(3-cyclopropylphenyl)-5-fluoropyrimidin-2-yl)amino)cyclohexyl)-4-(4-(4-((2,6-dioxopiperidin-3-yl)amino)-2-fluorophenyl)piperazin-1-yl)-N-methylcyclohexane-1-carboxamide (Compound 478)

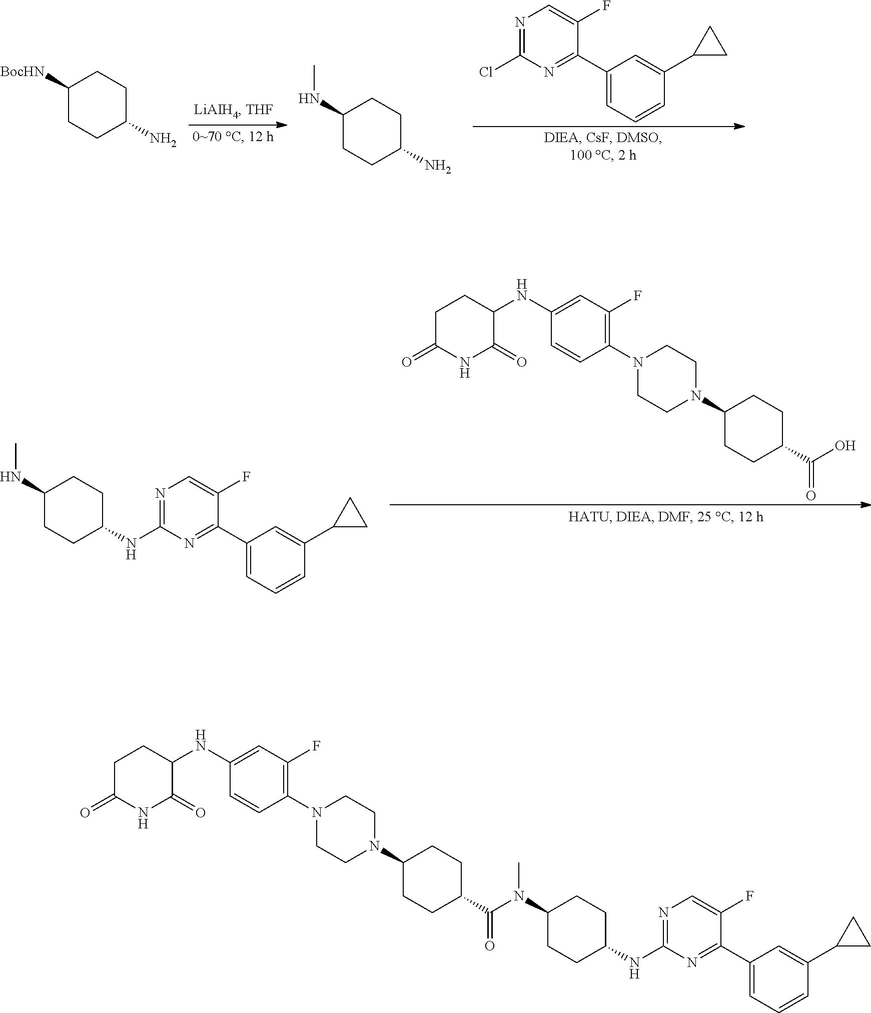

Synthesis of trans-N1-methylcyclohexane-1,4-diamine. To a solution of trans-tert-butyl N-(4-aminocyclohexyl)carbamate (10.0 g, 46.6 mmol, 1.00 eq) in THF (60 mL) was added LiAlH₄ (2.50 M, 93.3 mL, 5.00 eq) at 0 °C. The mixture was stirred at 70 °C for 12 h. LC-MS showed trans-tert-butyl N-(4-aminocyclohexyl)carbamate was consumed completely and the desired mass was detected. The reaction was quenched by H₂O (10 g), 15% NaOH solution (10 g) and H₂O (30 g) at 0 °C, the mixture was dried over Na₂SO₄, filtered and concentrated under reduced pressure to give the title compound (5.00 g, crude) as a brown oil. LCMS (ES⁺): m/z 129.4 [M+H]⁺.

Synthesis of trans-N-(4-(3-cyclopropylphenyl)-5-fluoropyrimidin-2-yl)-N4-methylcyclohexane-1,4-diamine. To a solution of trans-N1-methylcyclohexane-1,4-diamine (500 mg, 3.90 mmol, 1.00 eq) in DMSO (20 mL) was added CsF (592 mg, 3.90 mmol, 143 μL, 1.00 eq), 2-chloro-4-(3-cyclopropylphenyl)-5-fluoro-pyrimidine (484 mg, 1.95 mmol, 0.500 eq) and DIEA (1.51 g, 11.7 mmol, 2.04 mL, 3.00 eq) at 25 °C. The mixture was stirred at 100 °C for 2 h. LC-MS showed trans-N1-methylcyclohexane-1,4-diamine was consumed completely and the desired mass was detected. The reaction was quenched by addition H₂O 80 mL at 25 °C, and then diluted with H₂O 40 mL and then extracted with EtOAc (100 mL*3). The combined organic layers were washed with brine (50 mL*3), dried over Na₂SO₄, filtered and concentrated under reduced pressure. The residue was purified by column chromatography (SiO2, Petroleum ether:Ethyl acetate=3:1 to 0:1) to give the title compound (300 mg, 763 μmol, 19.5% yield, 86.6% purity) as a light yellow solid. LCMS (ES⁺): m/z 341.2 [M+H]⁺.

Synthesis of trans-N-((trans)-4-((4-(3-cyclopropylphenyl)-5-fluoropyrimidin-2-yl)amino)cyclohexyl)-4-(4-(4-((2,6-dioxopiperidin-3-yl)amino)-2-fluorophenyl)piperazin-1-yl)-N-methylcyclohexane-1-carboxamide (Compound 478). To a solution of trans-N1-(4-(3-cyclopropylphenyl)-5-fluoropyrimidin-2-yl)-N4-methylcyclohexane-1,4-diamine (150 mg, 440 μmol, 1.00 eq) in DMF (3 mL) was added HATU (167 mg, 440 μmol, 1.00 eq), 4-[4-[4-[(2,6-dioxo-3-piperidyl)amino]-2-fluoro-phenyl]piperazin-1-yl]cyclohexanecarboxylic acid (190 mg, 348 μmol, 7.91 eq, TFA) and DIEA (170 mg, 1.32 mmol, 230 μL, 3.00 eq) at 25 °C. The mixture was stirred at 25 °C for 12 h. LC-MS showed trans-N1-(4-(3-cyclopropylphenyl)-5-fluoropyrimidin-2-yl)-N4-methylcyclohexane-1,4-diamine was consumed completely and the desired mass was detected. The reaction mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 25%-55% B over 8.0 min) to give the title compound (Compound 478) (50.4 mg, 66.5 μmol, 15.1% yield, 99.6% purity, 0.260 FA) as a pale yellow solid. LCMS (ES⁺): m/z 755.5 [M+H]⁺.

### Example 568. Synthesis of trans-(1r,4R)—N-((1r,4R)-4-((4-(3-cyclopropylphenyl)-5-fluoropyrimidin-2-yl)amino)cyclohexyl)-4-(4-(4-((2,6-dioxopiperidin-3-yl)amino)-2-fluorophenyl)piperidin-1-yl)cyclohexane-1-carboxamide (Compound 558)

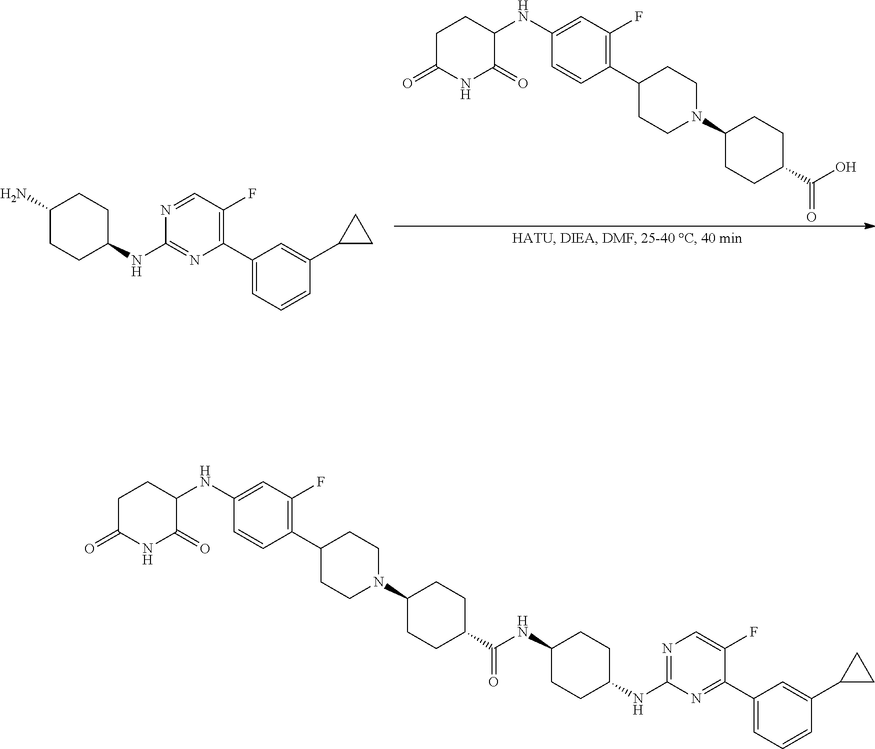 [SMILES: N[C@H]1CC[C@H](Nc2ncc(F)c(-c3cccc(C4CC4)c3)n2)CC1 ; O=C1CCC(Nc2ccc(C3CCN([C@H]4CC[C@H](C(=O)O)CC4)CC3)c(F)c2)C(=O)N1 ; O=C1CCC(Nc2ccc(C3CCN([C@H]4CC[C@H](C(=O)N[C@H]5CC[C@H](Nc6ncc(F)c(-c7cccc(C8CC8)c7)n6)CC5)CC4)CC3)c(F)c2)C(=O)N1]

Synthesis of trans-(1r,4R)—N-((1r,4R)-4-((4-(3-cyclopropylphenyl)-5-fluoropyrimidin-2-yl)amino)cyclohexyl)-4-(4-(4-((2,6-dioxopiperidin-3-yl)amino)-2-fluorophenyl)piperidin-1-yl)cyclohexane-1-carboxamide (Compound 558). To a solution of trans-4-[4-[4-[(2,6-dioxo-3-piperidyl)amino]-2-fluoro-phenyl]-1-piperidyl]cyclohexanecarboxylic acid (170 mg, 394 μmol, 1.00 eq) in DMF (4 mL) were added HATU (150 mg, 394 μmol, 1.00 eq) and DIEA (255 mg, 1.97 mmol, 343 μL, 5.00 eq) at 25 °C, the mixture was stirred at 25 °C for 10 min. Then trans-N1-(4-(3-cyclopropylphenyl)-5-fluoropyrimidin-2-yl)cyclohexane-1,4-diamine (104 mg, 236 μmol, 0.600 eq, TFA) was added at 25 °C, the mixture was stirred at 40 °C for 30 min. LCMS showed that the trans-4-[4-[4-[(2,6-dioxo-3-piperidyl)amino]-2-fluoro-phenyl]-1-piperidyl]cyclohexanecarboxylic acid was consumed and the desired mass was detected. To the mixture was added H₂O (5 mL) and the mixture was extracted with EtOAc (5 mL*3). The combined organic layers were washed with brine (8 mL), dried over Na₂SO₄, filtered and the filtrate was concentrated under reduced pressure to give the crude product. The crude product was purified by prep-HPLC (column: Phenomenex luna C18 100*40 mm*3 um; mobile phase: [H₂O (0.2% FA)-ACN]; gradient: 15%-55% B over 8.0 min) to give the title compound (Compound 558) (50.7 mg, 64.2 μmol, 16.3% yield, 93.7% purity) as a pale yellow solid. LCMS (ES⁺): m/s 740.4 [M+H]⁺

### Example 569. Synthesis of N-((1r,4r)-4-((4-(3-cyclopropylphenyl)-5-fluoropyrimidin-2-yl)amino)cyclohexyl)-1′-(4-((2,6-dioxopiperidin-3-yl)amino)-2-fluorophenyl)-3′-fluoro-[1,4′-bipiperidine]-4-carboxamide (Compound 502)

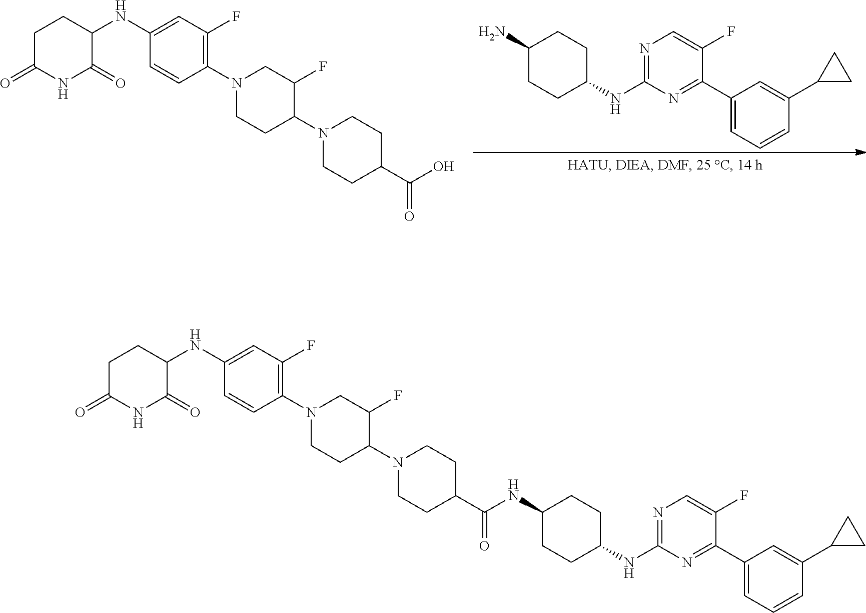 [SMILES: O=C1CCC(Nc2ccc(N3CCC(N4CCC(C(=O)O)CC4)C(F)C3)c(F)c2)C(=O)N1 ; N[C@H]1CC[C@H](Nc2ncc(F)c(-c3cccc(C4CC4)c3)n2)CC1 ; O=C1CCC(Nc2ccc(N3CCC(N4CCC(C(=O)N[C@H]5CC[C@H](Nc6ncc(F)c(-c7cccc(C8CC8)c7)n6)CC5)CC4)C(F)C3)c(F)c2)C(=O)N1]

Synthesis of N-((1r,4r)-4-((4-(3-cyclopropylphenyl)-5-fluoropyrimidin-2-yl)amino)cyclohexyl)-1′-(4-((2,6-dioxopiperidin-3-yl)amino)-2-fluorophenyl)-3′-fluoro-[1,4′-bipiperidine]-4-carboxamide (Compound 502). To a solution of 1′-(4-((2,6-dioxopiperidin-3-yl)amino)-2-fluorophenyl)-3′-fluoro-[1,4′-bipiperidine]-4-carboxylic acid (267 mg, 592 μmol, 1.00 eq) in DMF (5 mL) were added DIEA (383 mg, 2.95 mmol, 515 μL, 5.00 eq) and HATU (90.1 mg, 237 μmol, 0.400 eq) at 25 °C. Then to the mixture was added trans-N¹-(4-(3-cyclopropylphenyl)-5-fluoropyrimidin-2-yl)cyclohexane-1,4-diamine (183 mg, 415 μmol, 0.700 eq, TFA) at 25 °C. The mixture was stirred at 25 °C for 14 h. LCMS showed 1′-(4-((2,6-dioxopiperidin-3-yl)amino)-2-fluorophenyl)-3′-fluoro-[1,4′-bipiperidine]-4-carboxylic acid was consumed completely and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 40%-60% B over 8.0 min) to give the title compound (Compound 502) (54.0 mg, 65.6 μmol, 11.1% yield, 92.21% purity) as a yellow solid. LCMS (ES⁺): m/z 759.4 [M+H]⁺

### Example 570. Synthesis of trans-N-(4-((4-(3-cyclopropylphenyl)-5-fluoropyrimidin-2-yl)amino)cyclohexyl)-1′-(4-((2,6-dioxopiperidin-3-yl)amino)-2-fluorophenyl)-3′,3′-difluoro-[1,4′-bipiperidine]-4-carboxamide (Compound 497)

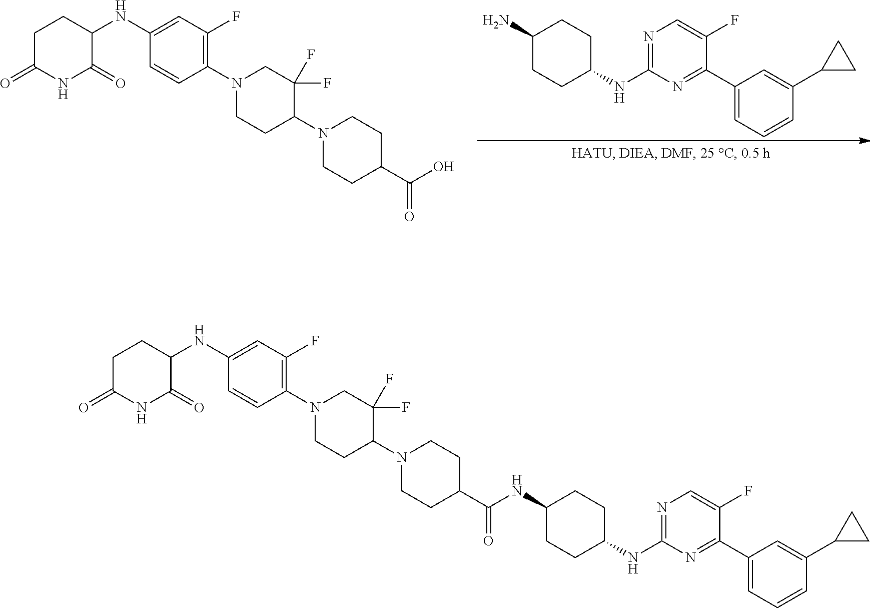 [SMILES: O=C1CCC(Nc2ccc(N3CCC(N4CCC(C(=O)O)CC4)C(F)(F)C3)c(F)c2)C(=O)N1 ; N[C@H]1CC[C@H](Nc2ncc(F)c(-c3cccc(C4CC4)c3)n2)CC1 ; O=C1CCC(Nc2ccc(N3CCC(N4CCC(C(=O)N[C@H]5CC[C@H](Nc6ncc(F)c(-c7cccc(C8CC8)c7)n6)CC5)CC4)C(F)(F)C3)c(F)c2)C(=O)N1]

Synthesis of trans-N-(4-((4-(3-cyclopropylphenyl)-5-fluoropyrimidin-2-yl)amino)cyclohexyl)-1′-(4-((2,6-dioxopiperidin-3-yl)amino)-2-fluorophenyl)-3′,3′-difluoro-[1,4′-bipiperidine]-4-carboxamide (Compound 497). To a solution of 1′-(4-((2,6-dioxopiperidin-3-yl)amino)-2-fluorophenyl)-3′,3′-difluoro-[1,4′-bipiperidine]-4-carboxylic acid (200 mg, 427 μmol, 1.00 eq) in DMF (7 mL) were added HATU (64.9 mg, 171 μmol, 0.400 eq), DIEA (276 mg, 2.13 mmol, 372 μL, 5.00 eq) and trans-N4-[4-(3-cyclopropylphenyl)-5-fluoro-pyrimidin-2-yl]cyclohexane-1,4-diamine (132 mg, 299 μmol, 0.700 eq, TFA) at 25 °C. The mixture was stirred at 25 °C for 0.5 h. LCMS showed 1′-(4-((2,6-dioxopiperidin-3-yl)amino)-2-fluorophenyl)-3′,3′-difluoro-[1,4′-bipiperidine]-4-carboxylic acid was consumed completely and the desired mass was detected. The reaction mixture was filtered and the filtrate was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 40%-70% B over 8.0 min) to give the title compound (Compound 497) (46.9 mg, 55.7 μmol, 13.1% yield, 92.3% purity) as a pale yellow solid. LCMS (ES⁺): m/z 777.4 [M+H]⁺.

### Example 571. Synthesis of trans-4-((4-(3-cyclopropylphenyl)-5-fluoropyrimidin-2-yl)amino)-N-(trans-4-(4-(4-((2,6-dioxopiperidin-3-yl)amino)-2-fluorophenyl)piperidin-1-yl)cyclohexyl)cyclohexane-1-carboxamide (Compound 503)

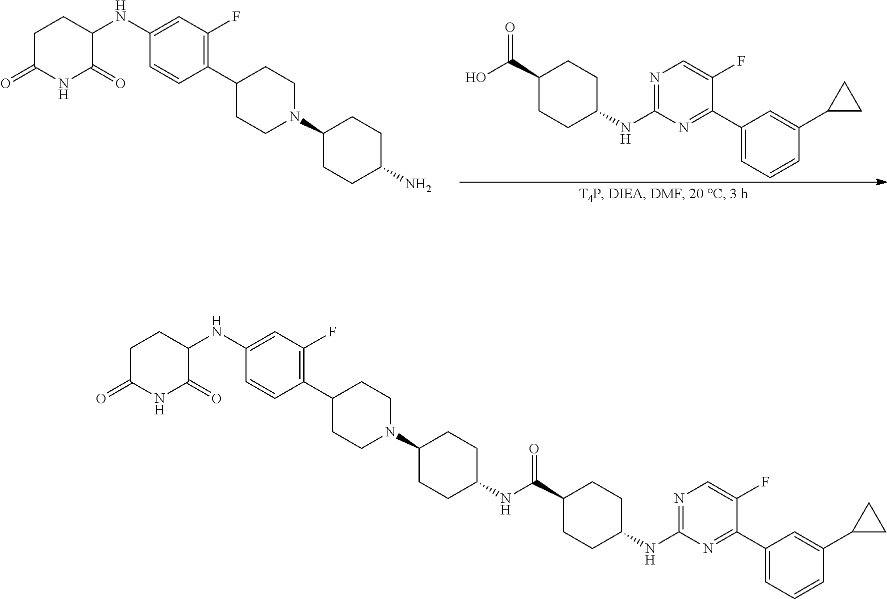 [SMILES: N[C@H]1CC[C@H](N2CCC(c3ccc(NC4CCC(=O)NC4=O)cc3F)CC2)CC1 ; O=C(O)[C@H]1CC[C@H](Nc2ncc(F)c(-c3cccc(C4CC4)c3)n2)CC1 ; O=C1CCC(Nc2ccc(C3CCN([C@H]4CC[C@H](NC(=O)[C@H]5CC[C@H](Nc6ncc(F)c(-c7cccc(C8CC8)c7)n6)CC5)CC4)CC3)c(F)c2)C(=O)N1]

Synthesis of trans-4-((4-(3-cyclopropylphenyl)-5-fluoropyrimidin-2-yl)amino)-N-(trans-4-(4-(4-((2,6-dioxopiperidin-3-yl)amino)-2-fluorophenyl)piperidin-1-yl)cyclohexyl)cyclohexane-1-carboxamide (Compound 503). To a solution of rac-3-((4-(1-((1r,4r)-4-aminocyclohexyl)piperidin-4-yl)-3-fluorophenyl)amino)piperidine-2,6-dione (187 mg, 362 μmol, 7.79e-1 eq, TFA) and trans-(1r,4r)-4-((4-(3-cyclopropylphenyl)-5-fluoropyrimidin-2-yl)amino)cyclohexane-1-carboxylic acid (165 mg, 464 μmol, 1.00 eq) in DMF (2 mL) were added DIEA (180 mg, 1.39 mmol, 243 μL, 3.00 eq) and T₄P (41 mg, 1.16 mmol, 2.50 eq) at 20 °C. The mixture was stirred at 20 °C for 3 h. LC-MS showed trans-3-((4-(1-((1r,4r)-4-aminocyclohexyl)piperidin-4-yl)-3-fluorophenyl)amino)piperidine-2,6-dione was consumed completely and desired mass was detected. The residue was diluted with H₂O (5 mL) and extracted with EtOAc (3 mL*5). The combined organic layers were washed with brine (5 mL), dried over anhydrous Na₂SO₄, filtered and concentrated under reduced pressure. The residue was purified by prep-HPLC (column: Phenomenex luna C18 100*40 mm*5 um; mobile phase: [H₂O (0.04% HCl)-ACN]; gradient: 30%-50% B over 8.0 min) to give the title compound (Compound 503) (23.2 mg, 29.9 μmol, 6.44% yield, 95.4% purity) as a green solid. LCMS (ES⁺): m/z 740.4 [M+H]⁺

### Example 572. Synthesis of trans-(1r,4R)-4-((4-(3-cyclopropylphenyl)-5-fluoropyrimidin-2-yl)amino)-N-((1r,4R)-4-(4-(4-((2,6-dioxopiperidin-3-yl)amino)-2-fluorophenyl)piperazin-1-yl)cyclohexyl)cyclohexane-1-carboxamide (Compound 557)

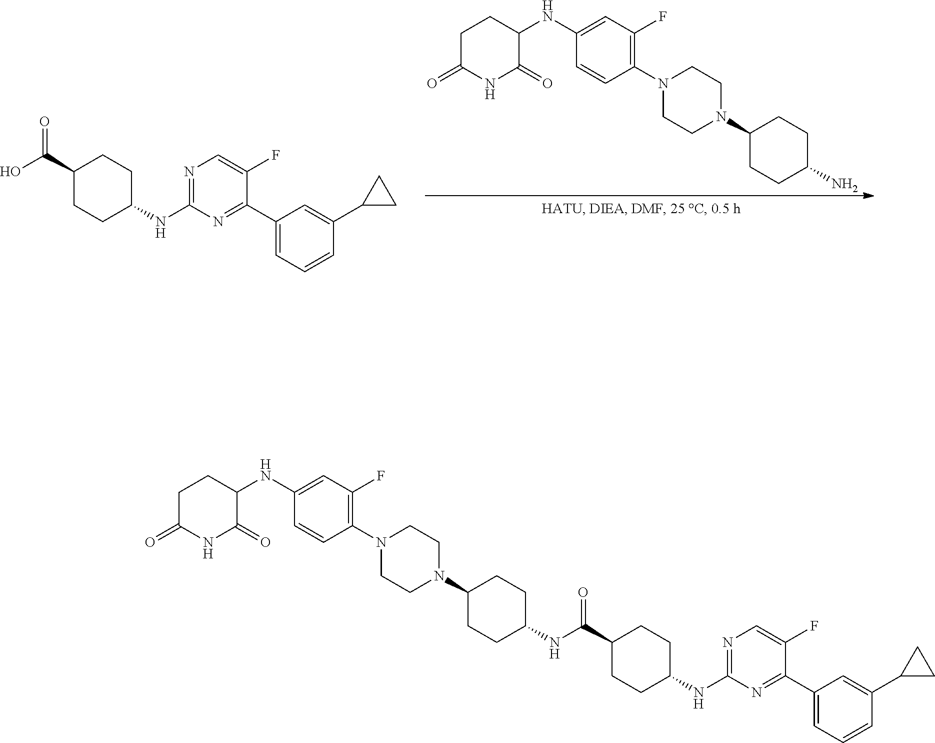 [SMILES: O=C(O)[C@H]1CC[C@H](Nc2ncc(F)c(-c3cccc(C4CC4)c3)n2)CC1 ; N[C@H]1CC[C@H](N2CCN(c3ccc(NC4CCC(=O)NC4=O)cc3F)CC2)CC1 ; O=C1CCC(Nc2ccc(N3CCN([C@H]4CC[C@H](NC(=O)[C@H]5CC[C@H](Nc6ncc(F)c(-c7cccc(C8CC8)c7)n6)CC5)CC4)CC3)c(F)c2)C(=O)N1]

Synthesis of trans-(1r,4R)-4-((4-(3-cyclopropylphenyl)-5-fluoropyrimidin-2-yl)amino)-N-((1r,4R)-4-(4-(4-((2,6-dioxopiperidin-3-yl)amino)-2-fluorophenyl)piperazin-1-yl)cyclohexyl)cyclohexane-1-carboxamide (Compound 557). To a solution of trans-4-((4-(3-cyclopropylphenyl)-5-fluoropyrimidin-2-yl)amino)cyclohexane-1-carboxylic acid (96.2 mg, 271 μmol, 0.500 eq) in DMF (3 mL) were added HATU (72.0 mg, 189 μmol, 0.350 eq), DIEA (350 mg, 2.71 mmol, 471 μL, 5.00 eq) and trans-3-((4-(4-(4-aminocyclohexyl)piperazin-1-yl)-3-fluorophenyl)amino)piperidine-2,6-dione (280 mg, 541 μmol, 1.00 eq, TFA) at 25 °C. The mixture was stirred at 25 °C for 0.5 h. LCMS showed trans-3-((4-(4-(4-aminocyclohexyl)piperazin-1-yl)-3-fluorophenyl)amino)piperidine-2,6-dione was consumed completely and the desired mass was detected. The reaction mixture was filtered and the filtrate was concentrated under reduced pressure to give a residue. The residue was purified by prep-HPLC (column: Waters Xbridge BEH C18 250*50 mm*10 um; mobile phase: [H₂O (10 mM NH₄HCO₃)-ACN]; gradient: 40%-70% B over 10.0 min) to give the title compound (Compound 557) (24.9 mg, 33.4 μmol, 6.17% yield, 99.3% purity) as a yellow solid. LCMS (ES⁺): m/z 741.5 [M+H]⁺.

### Example 573. Synthesis of trans-4-((4-(3-cyclopropylphenyl)-5-fluoropyrimidin-2-yl)amino)-N-(1′-(4-((2,6-dioxopiperidin-3-yl)amino)-2-fluorophenyl)-[1,4′-bipiperidin]-4-yl)cyclohexane-1-carboxamide (Compound 533)

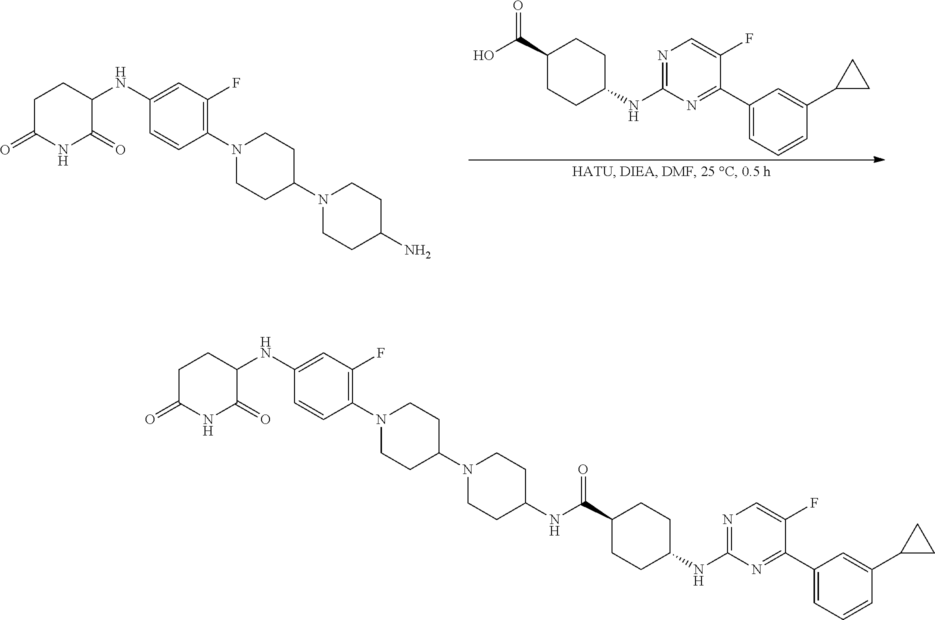 [SMILES: NC1CCN(C2CCN(c3ccc(NC4CCC(=O)NC4=O)cc3F)CC2)CC1 ; O=C(O)[C@H]1CC[C@H](Nc2ncc(F)c(-c3cccc(C4CC4)c3)n2)CC1 ; O=C1CCC(Nc2ccc(N3CCC(N4CCC(NC(=O)[C@H]5CC[C@H](Nc6ncc(F)c(-c7cccc(C8CC8)c7)n6)CC5)CC4)CC3)c(F)c2)C(=O)N1]

Synthesis of trans-4-((4-(3-cyclopropylphenyl)-5-fluoropyrimidin-2-yl)amino)-N-(1′-(4-((2,6-dioxopiperidin-3-yl)amino)-2-fluorophenyl)-[1,4′-bipiperidin]-4-yl)cyclohexane-1-carboxamide (Compound 533). To a solution of trans-4-[[4-(3-cyclopropylphenyl)-5-fluoro-pyrimidin-2-yl]amino]cyclohexanecarboxylic acid (74.1 mg, 208 μmol, 0.350 eq) in DMF (6 mL) were added HATU (79.3 mg, 208 μmol, 0.350 eq), DIEA (385 mg, 2.98 mmol, 520 μL, 5.00 eq) and 3-((4-(4-amino-[1,4′-bipiperidin]-1′-yl)-3-fluorophenyl)amino)piperidine-2,6-dione (308 mg, 596 μmol, 1.00 eq, TFA) at 25 °C. The mixture was stirred at 25 °C for 0.5 h. LCMS showed 3-((4-(4-amino-[1,4′-bipiperidin]-1′-yl)-3-fluorophenyl)amino)piperidine-2,6-dione was consumed completely and the desired mass was detected. The reaction mixture was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 20%-50% B over 8.0 min) to give the title compound (Compound 533) (18.0 mg, 24.1 μmol, 4.04% yield, 99.0% purity) as a pale yellow solid. LCMS (ES⁺): m/z 741.4 [M+H]⁺.

### Example 574. Synthesis of trans-3-((4-(4-((1-(4-((4-(3-cyclopropylphenyl)-5-fluoropyrimidin-2-yl)amino)cyclohexane-1-carbonyl)-4-hydroxypiperidin-4-yl)methyl)piperazin-1-yl)-3-fluorophenyl)amino)piperidine-2,6-dione (Compound 492)

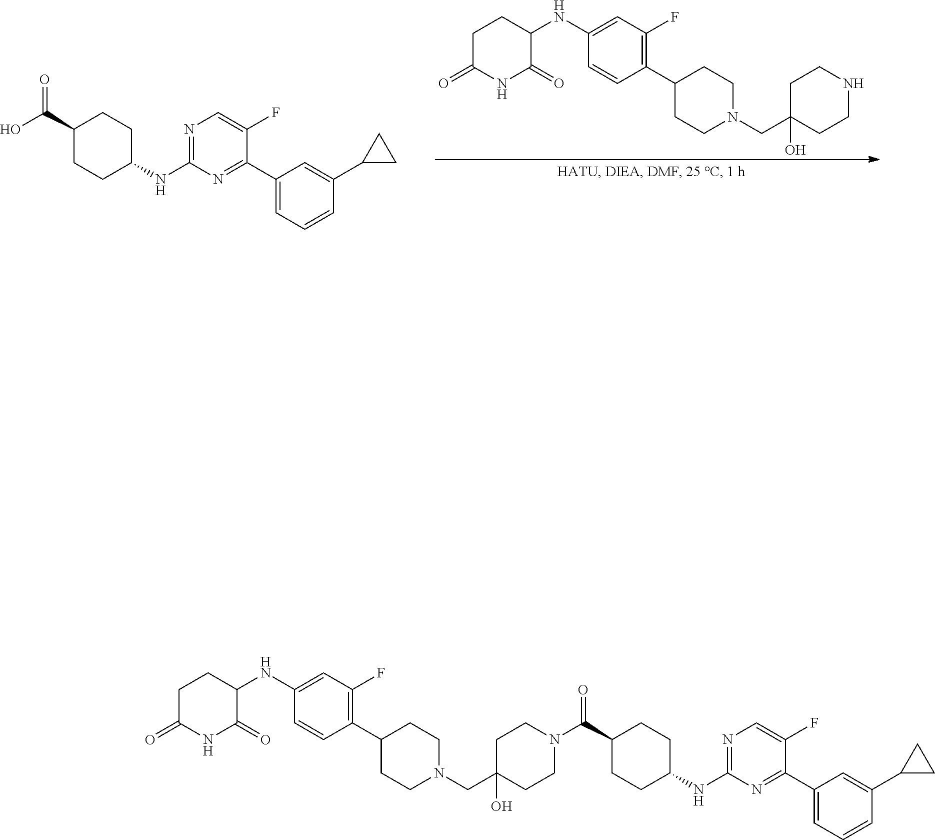 [SMILES: O=C(O)[C@H]1CC[C@H](Nc2ncc(F)c(-c3cccc(C4CC4)c3)n2)CC1 ; O=C1CCC(Nc2ccc(C3CCN(CC4(O)CCNCC4)CC3)c(F)c2)C(=O)N1 ; O=C1CCC(Nc2ccc(C3CCN(CC4(O)CCN(C(=O)[C@H]5CC[C@H](Nc6ncc(F)c(-c7cccc(C8CC8)c7)n6)CC5)CC4)CC3)c(F)c2)C(=O)N1]

Synthesis of trans-3-((4-(4-((1-(4-((4-(3-cyclopropylphenyl)-5-fluoropyrimidin-2-yl)amino)cyclohexane-1-carbonyl)-4-hydroxypiperidin-4-yl)methyl)piperazin-1-yl)-3-fluorophenyl)amino)piperidine-2,6-dione (Compound 492). To a solution of trans-4-((4-(3-cyclopropylphenyl)-5-fluoropyrimidin-2-yl)amino)cyclohexane-1-carboxylic (300 mg, 844 mol, 1.00 eq) in DMF (2 mL) was added DIEA (218 mg, 1.69 mmol, 294 μL, 2.00 eq), 3-((3-fluoro-4-(4-((4-hydroxypiperidin-4-yl)methyl)piperazin-1-yl)phenyl)amino)piperidine-2,6-dione (354 mg, 844 μmol, 1.00 eq) and HATU (641 mg, 1.69 mmol, 2.00 eq) at 25 °C. The mixture was stirred at 25 °C for 1 h. LC-MS showed the desired mass was detected. The reaction mixture was quenched by addition H₂O 3 mL, and then extracted with EtOAc (3 mL*3). The combined organic layers were dried over Na₂SO₄, filtered and concentrated under reduced pressure. The residue was purified by prep-HPLC (column: Waters Xbridge BEH C18 100*30 mm*10 um; mobile phase: [H₂O (10 mM NH₄HCO₃)-ACN]; gradient: 33%-63% B over 8.0 min) to give the title compound (Compound 492) (37.2 mg, 49.1 μmol, 5.82% yield, 100% purity) as a pale yellow solid. LCMS (ES⁺): m/z 757.1 [M+H]⁺.

### Example 575. Synthesis of trans-3-((4-(1-((1-(4-((4-(3-cyclopropylphenyl)-5-fluoropyrimidin-2-yl)amino)cyclohexane-1-carbonyl)-4-hydroxypiperidin-4-yl)methyl)piperidin-4-yl)-3-fluorophenyl)amino)piperidine-2,6-dione (Compound 518)

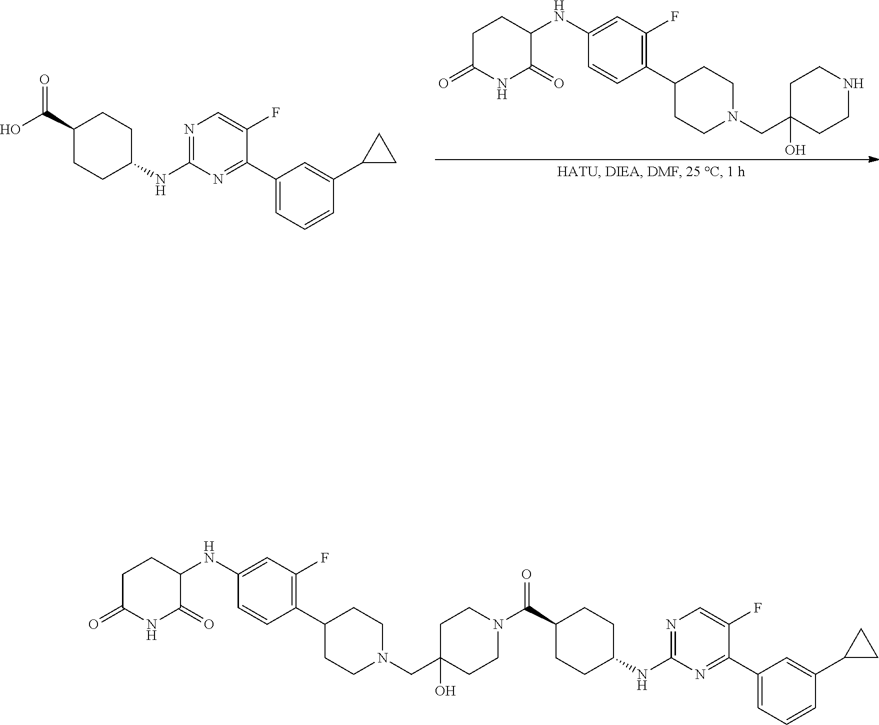 [SMILES: O=C(O)[C@H]1CC[C@H](Nc2ncc(F)c(-c3cccc(C4CC4)c3)n2)CC1 ; O=C1CCC(Nc2ccc(C3CCN(CC4(O)CCNCC4)CC3)c(F)c2)C(=O)N1 ; O=C1CCC(Nc2ccc(C3CCN(CC4(O)CCN(C(=O)[C@H]5CC[C@H](Nc6ncc(F)c(-c7cccc(C8CC8)c7)n6)CC5)CC4)CC3)c(F)c2)C(=O)N1]

Synthesis of trans-3-((4-(1-((1-(4-((4-(3-cyclopropylphenyl)-5-fluoropyrimidin-2-yl)amino)cyclohexane-1-carbonyl)-4-hydroxypiperidin-4-yl)methyl)piperidin-4-yl)-3-fluorophenyl)amino)piperidine-2,6-dione (Compound 518). To a solution of 3-((3-fluoro-4-(1-((4-hydroxypiperidin-4-yl)methyl)piperidin-4-yl)phenyl)amino)piperidine-2,6-dione (529 mg, 1.27 mmol, 1.00 eq) in DMF (2 mL) was added DIEA (490 mg, 3.80 mmol, 661 μL, 3.00 eq) until pH-7. To a solution of trans-4-((4-(3-cyclopropylphenyl)-5-fluoropyrimidin-2-yl)amino)cyclohexane-1-carboxylic acid (450 mg, 1.27 mmol, 1.00 eq) in DMF (2 mL) was added DIEA (490 mg, 3.80 mmol, 661 μL, 3.00 eq) until pH-7. Then HATU (722 mg, 1.90 mmol, 1.50 eq) was added to the mixture of the two solutions. The mixture was stirred at 25 °C for 1 h. LC-MS showed the desired mass was detected. The reaction mixture was filtered to give a residue. The residue was purified by prep-HPLC (column: Phenomenex luna C18 100*40 mm*5 um; mobile phase: [H₂O (0.2% FA)-ACN]; gradient: 20%-60% B over 8.0 min) to give the title compound (Compound 518) (204 mg, 256 μmol, 20.2% yield, 94.9% purity) as a green solid. LCMS (ES⁺): m/z 756.0 [M+H]⁺.

### Example 576. Synthesis of trans-3-((4-(4-((1-((1r,4r)-4-((4-(3-cyclopropylphenyl)-5-fluoropyrimidin-2-yl)amino)cyclohexane-1-carbonyl)-4-fluoropiperidin-4-yl)methyl)piperazin-1-yl)-3-fluorophenyl)amino)piperidine-2,6-dione (Compound 529)

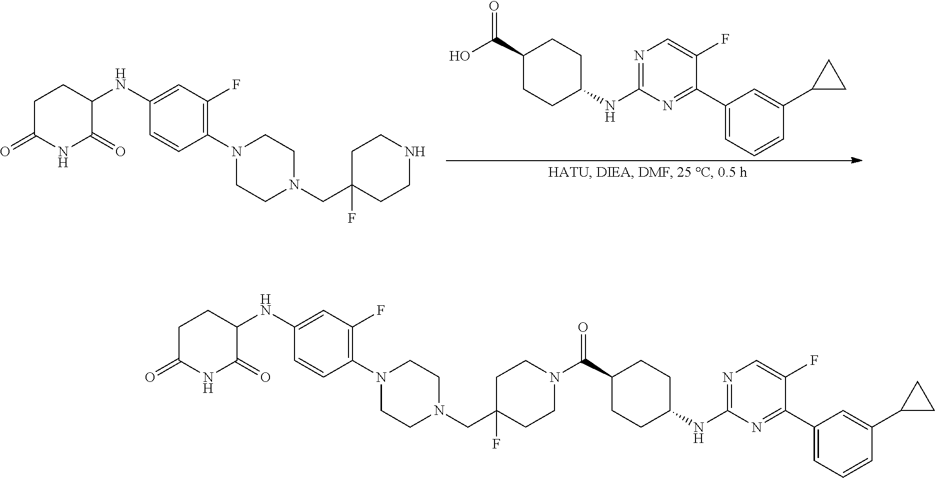 [SMILES: O=C1CCC(Nc2ccc(N3CCN(CC4(F)CCNCC4)CC3)c(F)c2)C(=O)N1 ; O=C(O)[C@H]1CC[C@H](Nc2ncc(F)c(-c3cccc(C4CC4)c3)n2)CC1 ; O=C1CCC(Nc2ccc(N3CCN(CC4(F)CCN(C(=O)[C@H]5CC[C@H](Nc6ncc(F)c(-c7cccc(C8CC8)c7)n6)CC5)CC4)CC3)c(F)c2)C(=O)N1]

Synthesis of trans-3-((4-(4-((1-(4-((4-(3-cyclopropylphenyl)-5-fluoropyrimidin-2-yl)amino)cyclohexane-1-carbonyl)-4-fluoropiperidin-4-yl)methyl)piperazin-1-yl)-3-fluorophenyl)amino)piperidine-2,6-dione (Compound 529). To a solution of trans-4-((4-(3-cyclopropylphenyl)-5-fluoropyrimidin-2-yl)amino)cyclohexane-1-carboxylic acid (199 mg, 560 μmol, 0.600 eq) in DMF (5 mL) were added HATU (124 mg, 327 μmol, 0.350 eq) and DIEA (603 mg, 4.67 mmol, 813 μL, 5.00 eq) at 25 °C, then to the mixture was added 3-[3-fluoro-4-[4-[(4-fluoro-4-piperidyl)methyl]piperazin-1-yl]anilino]piperidine-2,6-dione (500 mg, 934 μmol, 1.00 eq, TFA) at 25 °C. The resulting mixture was stirred at 25 °C for 0.5 h. LCMS showed 3-((3-fluoro-4-(4-((4-fluoropiperidin-4-yl)methyl)piperazin-1-yl)phenyl)amino)piperidine-2,6-dione was consumed and the desired target MS was detected. The mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 15%-45% B over 8.0 min) to give the title compound (Compound 529) (133 mg, 176 μmol, 18.8% yield, 100% purity) as a green solid. LCMS (ES⁺): m/z 759.4 [M+H]⁺

### Example 577. Synthesis of trans-3-[4-[4-[[1-[4-[[4-(3-cyclopropylphenyl)-5-fluoro-pyrimidin-2-yl]amino]cyclohexanecarbonyl]-4-piperidyl]methyl]piperazin-1-yl]-3-fluoro-anilino]piperidine-2,6-dione (Compound 555)

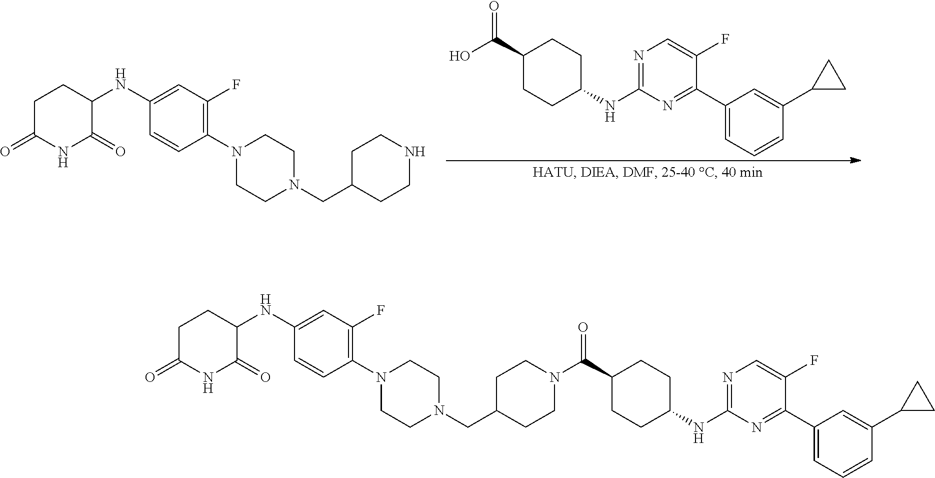 [SMILES: O=C1CCC(Nc2ccc(N3CCN(CC4CCNCC4)CC3)c(F)c2)C(=O)N1 ; O=C(O)[C@H]1CC[C@H](Nc2ncc(F)c(-c3cccc(C4CC4)c3)n2)CC1 ; O=C1CCC(Nc2ccc(N3CCN(CC4CCN(C(=O)[C@H]5CC[C@H](Nc6ncc(F)c(-c7cccc(C8CC8)c7)n6)CC5)CC4)CC3)c(F)c2)C(=O)N1]

Synthesis of trans-3-[4-[4-[[1-[4-[[4-(3-cyclopropylphenyl)-5-fluoro-pyrimidin-2-yl]amino]cyclohexanecarbonyl]-4-piperidyl]methyl]piperazin-1-yl]-3-fluoro-anilino]piperidine-2,6-dione (Compound 555). To a solution of trans-4-[[4-(3-cyclopropylphenyl)-5-fluoro-pyrimidin-2-yl]amino]cyclohexanecarboxylic acid (206 mg, 580 μmol, 1.00 eq) in DMF (5 mL) were added DIEA (375 mg, 2.9 mmol, 505 μL, 5.00 eq) and HATU (265 mg, 696 μmol, 1.20 eq), the mixture was stirred at 25 °C for 10 min, then 3-[3-fluoro-4-[4-(4-piperidylmethyl)piperazin-1-yl]anilino]piperidine-2,6-dione (300 mg, 580 μmol, 1.00 eq, TFA) was added at 25 °C, the mixture was stirred at 40 °C for 30 min. LCMS showed that trans-4-[[4-(3-cyclopropylphenyl)-5-fluoro-pyrimidin-2-yl]amino]cyclohexanecarboxylic acid was consumed and the desired mass was detected. To the mixture was added H₂O (8 mL) and the mixture was extracted with EtOAc (8 mL*3). The combined organic layers were washed with brine (10 mL*3), dried over Na₂SO₄, filtered and the filtrate was concentrated under reduced pressure to give the crude product. This mixture was purified by acidic prep-HPLC (column: Phenomenex luna C18 100*40 mm*3 um; mobile phase: [H₂O (0.2% FA)-ACN]; gradient: 15%-55% B over 8.0 min) to give the title compound (Compound 555) (215 mg, 268 μmol, 46.3% yield, 98.1% purity, FA) as a pale yellow solid. LCMS (ES⁺): m/z 741.3 [M+H]⁺.

### Example 578. Synthesis of trans-3-((4-(1-((1-(4-((4-(3-cyclopropylphenyl)-5-fluoropyrimidin-2-yl)amino)cyclohexane-1-carbonyl)piperidin-4-yl)methyl)piperidin-4-yl)-3-fluorophenyl)amino)piperidine-2,6-dione (Compound 554)

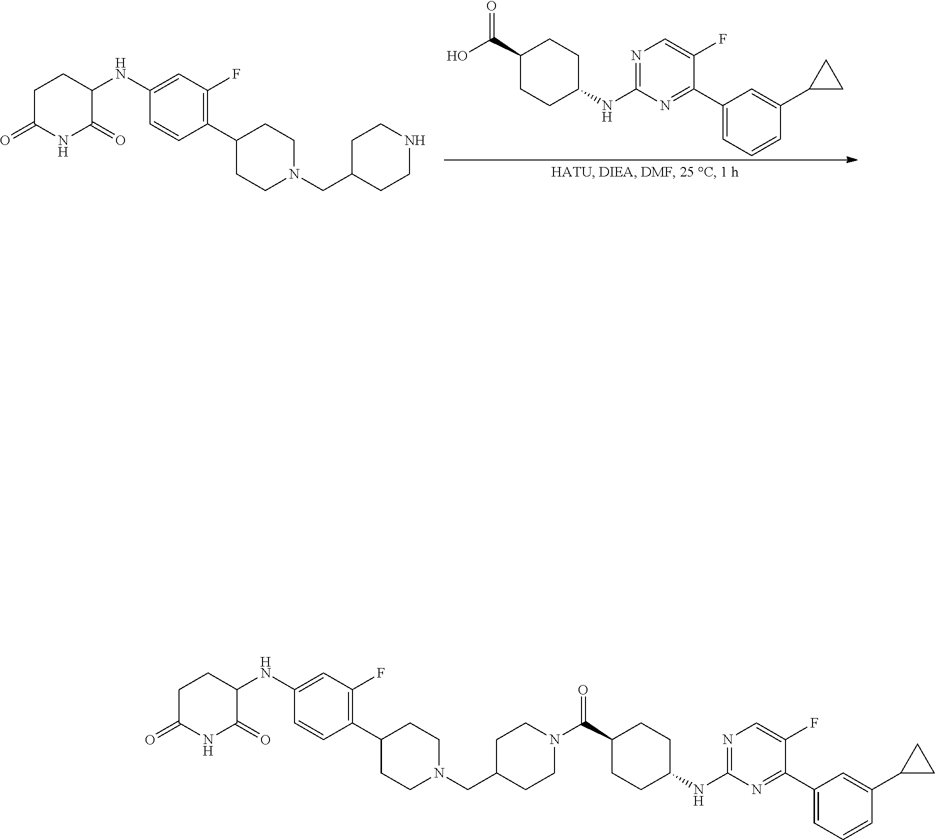 [SMILES: O=C1CCC(Nc2ccc(C3CCN(CC4CCNCC4)CC3)c(F)c2)C(=O)N1 ; O=C(O)[C@H]1CC[C@H](Nc2ncc(F)c(-c3cccc(C4CC4)c3)n2)CC1 ; O=C1CCC(Nc2ccc(C3CCN(CC4CCN(C(=O)[C@H]5CC[C@H](Nc6ncc(F)c(-c7cccc(C8CC8)c7)n6)CC5)CC4)CC3)c(F)c2)C(=O)N1]

Synthesis of trans-3-((4-(1-((1-(4-((4-(3-cyclopropylphenyl)-5-fluoropyrimidin-2-yl)amino)cyclohexane-1-carbonyl)piperidin-4-yl)methyl)piperidin-4-yl)-3-fluorophenyl)amino)piperidine-2,6-dione (Compound 554). To a solution of trans-4-((4-(3-cyclopropylphenyl)-5-fluoropyrimidin-2-yl)amino)cyclohexane-1-carboxylic acid (251 mg, 707 μmol, 0.500 eq) in DMF (8 mL) were added HATU (161 mg, 424 μmol, 0.300 eq), DIEA (913 mg, 7.07 mmol, 1.23 mL, 5.00 eq) and 3-((3-fluoro-4-(1-(piperidin-4-ylmethyl)piperidin-4-yl)phenyl)amino)piperidine-2,6-dione (730 mg, 1.41 mmol, 1.00 eq, TFA) at 25 °C. The mixture was stirred at 25 °C for 1 h. LCMS showed the desired mass was detected. The reaction mixture was filtered and the filtrate was concentrated under reduced pressure to give a residue. The residue was purified by prep-HPLC (column: Waters Xbridge BEH C18 250*50 mm*10 um; mobile phase: [H₂O (10 mM NH₄HCO₃)-ACN]; gradient: 45%-75% B over 10.0 min) to give the title compound (Compound 554) (80.9 mg, 107 μmol, 7.56% yield, 97.8% purity) as a white solid. LCMS (ES⁺): m/z 740.4 [M+H]⁺.

### Example 579. Synthesis of trans-4-(4-(4-((2,6-dioxopiperidin-3-yl)amino)-2-fluorophenyl)piperazin-1-yl)-N-(trans-4-((5-fluoro-4-(3-(piperidin-1-yl)phenyl)pyrimidin-2-yl)amino)cyclohexyl)cyclohexane-1-carboxamide (Compound 541)

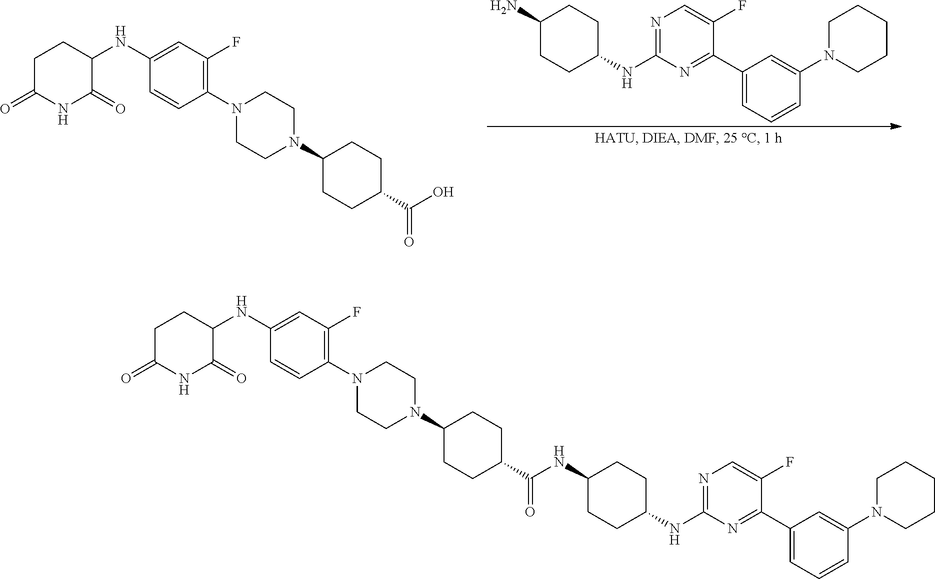 [SMILES: O=C1CCC(Nc2ccc(N3CCN([C@H]4CC[C@H](C(=O)O)CC4)CC3)c(F)c2)C(=O)N1 ; N[C@H]1CC[C@H](Nc2ncc(F)c(-c3cccc(N4CCCCC4)c3)n2)CC1 ; O=C1CCC(Nc2ccc(N3CCN([C@H]4CC[C@H](C(=O)N[C@H]5CC[C@H](Nc6ncc(F)c(-c7cccc(N8CCCCC8)c7)n6)CC5)CC4)CC3)c(F)c2)C(=O)N1]

Synthesis of trans-4-(4-(4-((2,6-dioxopiperidin-3-yl)amino)-2-fluorophenyl)piperazin-1-yl)-N-(trans-4-((5-fluoro-4-(3-(piperidin-1-yl)phenyl)pyrimidin-2-yl)amino)cyclohexyl)cyclohexane-1-carboxamide (Compound 541). To a solution of trans-4-(4-(4-((2,6-dioxopiperidin-3-yl)amino)-2-fluorophenyl)piperazin-1-yl)cyclohexane-1-carboxylic acid (300 mg, 693 μmol, 1.00 eq, TFA) and trans-N1-(5-fluoro-4-(3-(piperidin-1-yl)phenyl)pyrimidin-2-yl)cyclohexane-1,4-diamine (256 mg, 693 μmol, 1.00 eq, TFA) in DMF (5 mL) was added DIEA (179 mg, 1.39 mmol, 241 μL, 2.00 eq) and HATU (527 mg, 1.39 mmol, 2.00 eq) at 25 °C. The mixture was stirred at 25 °C for 1 h. LC-MS showed the desired mass was detected. The reaction mixture was filtered and concentrated under reduced pressure. The residue was purified by prep-HPLC (column: Phenomenex luna C18 100*40 mm*3 um; mobile phase: [H₂O (0.2% FA)-ACN]; gradient: 5%-35% B over 8.0 min) to give the title compound (Compound 541) (154 mg, 196 μmol, 28.3% yield, 100% purity) as a white solid. LCMS (ES⁺): m/z 784.5 [M+H]⁺.

### Example 580. Synthesis of trans-1′-(4-((2,6-dioxopiperidin-3-yl)amino)-2-fluorophenyl)-N-(4-((5-fluoro-4-(3-(piperidin-1-yl)phenyl)pyrimidin-2-yl)amino)cyclohexyl)-[1,4′-bipiperidine]-4-carboxamide (Compound 516)

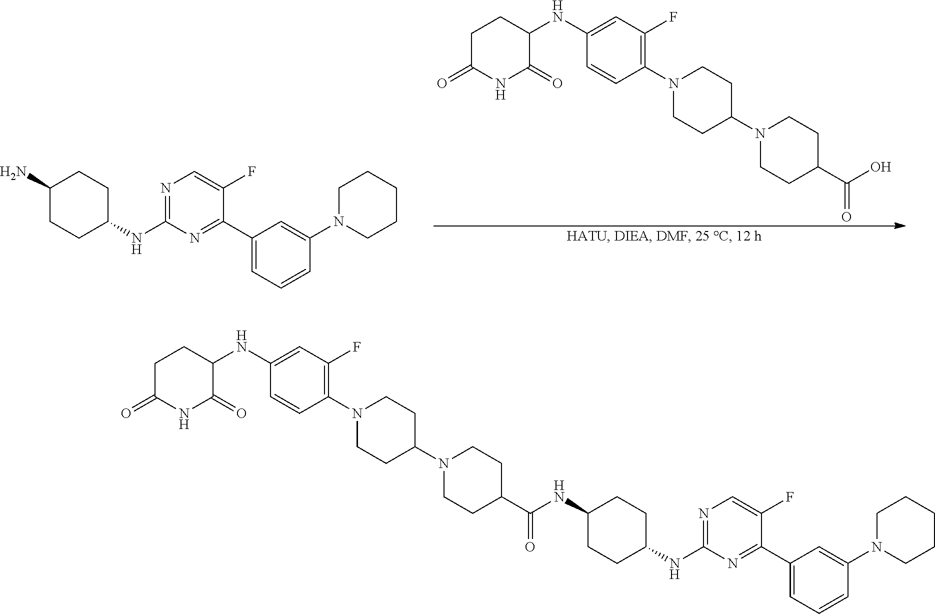 [SMILES: N[C@H]1CC[C@H](Nc2ncc(F)c(-c3cccc(N4CCCCC4)c3)n2)CC1 ; O=C1CCC(Nc2ccc(N3CCC(N4CCC(C(=O)O)CC4)CC3)c(F)c2)C(=O)N1 ; O=C1CCC(Nc2ccc(N3CCC(N4CCC(C(=O)N[C@H]5CC[C@H](Nc6ncc(F)c(-c7cccc(N8CCCCC8)c7)n6)CC5)CC4)CC3)c(F)c2)C(=O)N1]

Synthesis of trans-1′-(4-((2,6-dioxopiperidin-3-yl)amino)-2-fluorophenyl)-N-(4-((5-fluoro-4-(3-(piperidin-1-yl)phenyl)pyrimidin-2-yl)amino)cyclohexyl)-[1,4′-bipiperidine]-4-carboxamide (Compound 516). To a solution of 1-[1-[4-[(2,6-dioxo-3-piperidyl)amino]-2-fluoro-phenyl]-4-piperidyl]piperidine-4-carboxylic acid (319 mg, 739 μmol, 1.00 eq, TFA) in DMF (1 mL) was added DIEA (95.5 mg, 739 μmol, 128 μL, 1.00 eq) until pH~7. To a solution of trans-N¹-[5-fluoro-4-[3-(1-piperidyl)phenyl]pyrimidin-2-yl]cyclohexane-1,4-diamine (300 mg, 739 μmol, 1.00 eq, HCl) in DMF (2 mL) was added DIEA (95.5 mg, 739 mol, 128 μL, 1.00 eq) until pH~7. Then HATU (281 mg, 739 μmol, 1.00 eq) was added to the mixture of the two solutions. The mixture was stirred at 25 °C for 12 h. LC-MS showed the desired mass was detected. The reaction mixture was filtered. The residue was purified by prep-HPLC (column: Phenomenex luna C18 100*40 mm*5 um; mobile phase: [H₂O (0.2% FA)-ACN]; gradient: 5%-45% B over 8.0 min) to give the title compound (Compound 516) (168 mg, 208 μmol, 28.1% yield, 99.7% purity, 0.5 FA) as a pale yellow solid. LCMS (ES⁺): m/z 784.5 [M+H]⁺.

### Example 581. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(3-(piperidin-1-yl)phenyl)pyrimidin-2-yl)amino)cyclohexyl)cyclohexane-1-carboxamide (Compound 556)

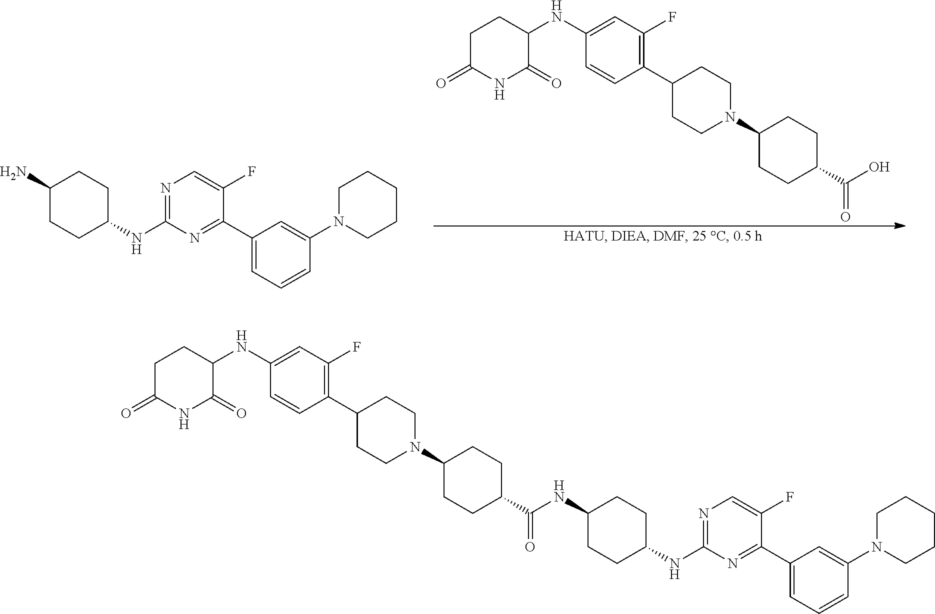 [SMILES: N[C@H]1CC[C@H](Nc2ncc(F)c(-c3cccc(N4CCCCC4)c3)n2)CC1 ; O=C1CCC(Nc2ccc(C3CCN([C@H]4CC[C@H](C(=O)O)CC4)CC3)c(F)c2)C(=O)N1 ; O=C1CCC(Nc2ccc(C3CCN([C@H]4CC[C@H](C(=O)N[C@H]5CC[C@H](Nc6ncc(F)c(-c7cccc(N8CCCCC8)c7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(3-(piperidin-1-yl)phenyl)pyrimidin-2-yl)amino)cyclohexyl)cyclohexane-1-carboxamide (Compound 556). To a solution of trans-4-(4-(4-((2,6-dioxopiperidin-3-yl)amino)-2-fluorophenyl)piperidin-1-yl)cyclohexane-1-carboxylic acid (221 mg, 512 μmol, 1.00 eq) in DMF (7 mL) were added HATU (58.4 mg, 154 μmol, 0.300 eq), DIEA (332 mg, 2.57 mmol, 447 μL, 5.00 eq) and trans-N1-(5-fluoro-4-(3-(piperidin-1-yl)phenyl)pyrimidin-2-yl)cyclohexane-1,4-diamine (124 mg, 256 μmol, 0.500 eq, TFA) at 25 °C. The mixture was stirred at 25 °C for 0.5 h. LC-MS showed trans-N1-(5-fluoro-4-(3-(piperidin-1-yl)phenyl)pyrimidin-2-yl)cyclohexane-1,4-diamine was consumed completely the desired mass was detected. The reaction mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 5%-40% B over 8.0 min) to give the title compound (Compound 556) (66.5 mg, 77.8 μmol, 15.2% yield, 91.6% purity) as a pale yellow solid. LCMS (ES⁺): m/z 783.5 [M+H]⁺

### Example 582. Synthesis of 1′-(4-((2,6-dioxopiperidin-3-yl)amino)-2-fluorophenyl)-3′-fluoro-N-((1r,4r)-4-((5-fluoro-4-(3-(piperidin-1-yl)phenyl)pyrimidin-2-yl)amino)cyclohexyl)-[1,4′-bipiperidine]-4-carboxamide (Compound 523)

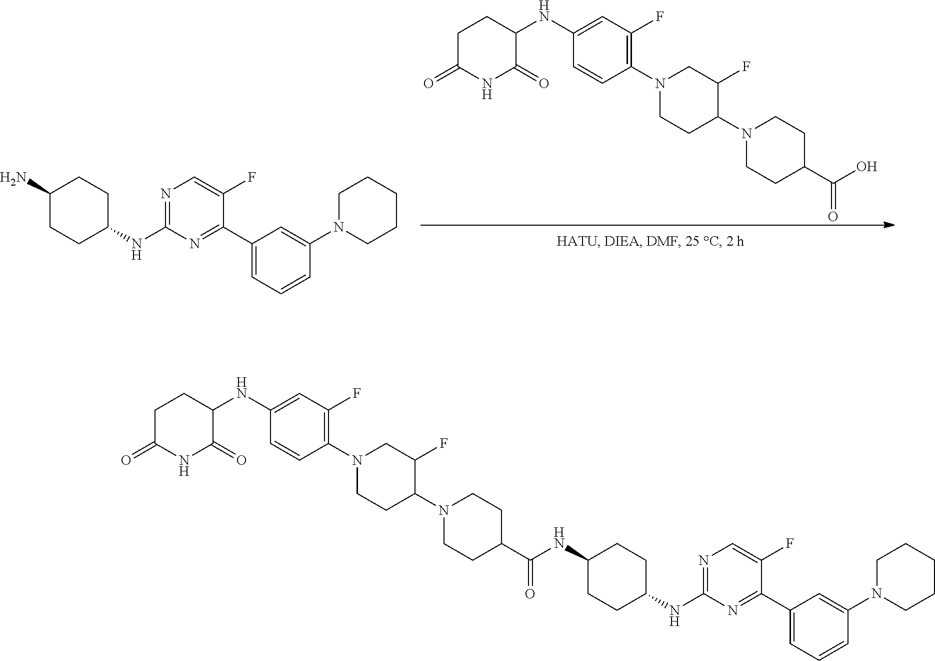 [SMILES: N[C@H]1CC[C@H](Nc2ncc(F)c(-c3cccc(N4CCCCC4)c3)n2)CC1 ; O=C1CCC(Nc2ccc(N3CCC(N4CCC(C(=O)O)CC4)C(F)C3)c(F)c2)C(=O)N1 ; O=C1CCC(Nc2ccc(N3CCC(N4CCC(C(=O)N[C@H]5CC[C@H](Nc6ncc(F)c(-c7cccc(N8CCCCC8)c7)n6)CC5)CC4)C(F)C3)c(F)c2)C(=O)N1]

Synthesis of 1′-(4-((2,6-dioxopiperidin-3-yl)amino)-2-fluorophenyl)-3′-fluoro-N-((1r,4r)-4-((5-fluoro-4-(3-(piperidin-1-yl)phenyl)pyrimidin-2-yl)amino)cyclohexyl)-[1,4′-bipiperidine]-4-carboxamide (Compound 523). To a solution of 1′-(4-((2,6-dioxopiperidin-3-yl)amino)-2-fluorophenyl)-3′-fluoro-[1,4′-bipiperidine]-4-carboxylic acid (300 mg, 666 μmol, 1.00 eq) in DMF (6 mL) were added HATU (177 mg, 466 μmol, 0.700 eq) and DIEA (430.35 mg, 3.33 mmol, 579.99 μL, 5.00 eq) at 25 °C, and then trans-N1-(5-fluoro-4-(3-(piperidin-1-yl)phenyl)pyrimidin-2-yl)cyclohexane-1,4-diamine (189 mg, 466 μmol, 0.700 eq, HCl) was added at 25 °C. The resulting mixture was stirred at 25 °C for 2 h. LCMS showed that 1′-(4-((2,6-dioxopiperidin-3-yl)amino)-2-fluorophenyl)-3′-fluoro-[1,4′-bipiperidine]-4-carboxylic acid was consumed completely and the desired mass was detected. The mixture was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 40%-65% B over 8.0 min) to give the title compound (Compound 523) (41.6 mg, 49.6 μmol, 7.45% yield, 95.7% purity) as a light yellow solid. LCMS (ESI+): m/s 802.5 [M+H]⁺

### Example 583. Synthesis of trans-1′-(4-((2,6-dioxopiperidin-3-yl)amino)-2-fluorophenyl)-3′,3′-difluoro-N-(4-((5-fluoro-4-(3-(piperidin-1-yl)phenyl)pyrimidin-2-yl)amino)cyclohexyl)-[1,4′-bipiperidine]-4-carboxamide (Compound 498)

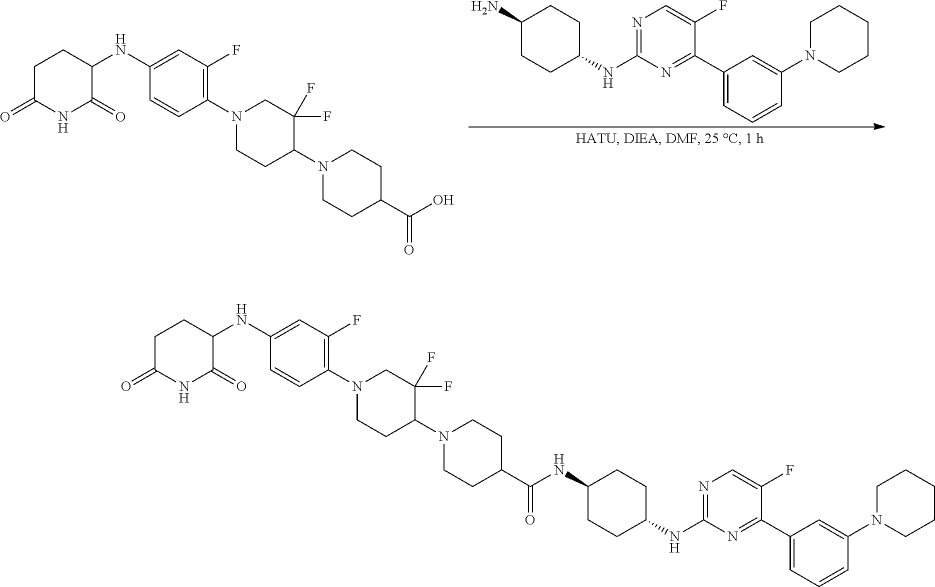 [SMILES: O=C1CCC(Nc2ccc(N3CCC(N4CCC(C(=O)O)CC4)C(F)(F)C3)c(F)c2)C(=O)N1 ; N[C@H]1CC[C@H](Nc2ncc(F)c(-c3cccc(N4CCCCC4)c3)n2)CC1 ; O=C1CCC(Nc2ccc(N3CCC(N4CCC(C(=O)N[C@H]5CC[C@H](Nc6ncc(F)c(-c7cccc(N8CCCCC8)c7)n6)CC5)CC4)C(F)(F)C3)c(F)c2)C(=O)N1]

Synthesis of trans-1′-(4-((2,6-dioxopiperidin-3-yl)amino)-2-fluorophenyl)-3′,3′-difluoro-N-(4-((5-fluoro-4-(3-(piperidin-1-yl)phenyl)pyrimidin-2-yl)amino)cyclohexyl)-[1,4′-bipiperidine]-4-carboxamide (Compound 498). To a solution of 1′-(4-((2,6-dioxopiperidin-3-yl)amino)-2-fluorophenyl)-3′,3′-difluoro-[1,4′-bipiperidine]-4-carboxylic acid (200 mg, 427 μmol, 1.00 eq) in DMF (6 mL) were added HATU (64.9 mg, 170 μmol, 0.400 eq), DIEA (277 mg, 2.13 mmol, 372 μL, 5.00 eq) and trans-N1-(5-fluoro-4-(3-(piperidin-1-yl)phenyl)pyrimidin-2-yl)cyclohexane-1,4-diamine (82.6 mg, 171 μmol, 0.400 eq, TFA) at 25 °C. The mixture was stirred at 25 °C for 1 h. LC-MS showed 1′-(4-((2,6-dioxopiperidin-3-yl)amino)-2-fluorophenyl)-3′,3′-difluoro-[1,4′-bipiperidine]-4-carboxylic acid was consumed completely and the desired mass was detected. The reaction mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 50%-90% B over 8.0 min) to give the title compound (Compound 498) (69.6 mg, 79.3 μmol, 18.6% yield, 93.4% purity) as a pale yellow solid. LCMS (ES⁺): m/z 820.5 [M+H]⁺

### Example 584. Synthesis of trans-N-(trans-4-(4-(4-((2,6-dioxopiperidin-3-yl)amino)-2-fluorophenyl)piperidin-1-yl)cyclohexyl)-4-((5-fluoro-4-(3-(piperidin-1-yl)phenyl)pyrimidin-2-yl)amino)cyclohexane-1-carboxamide (Compound 515)

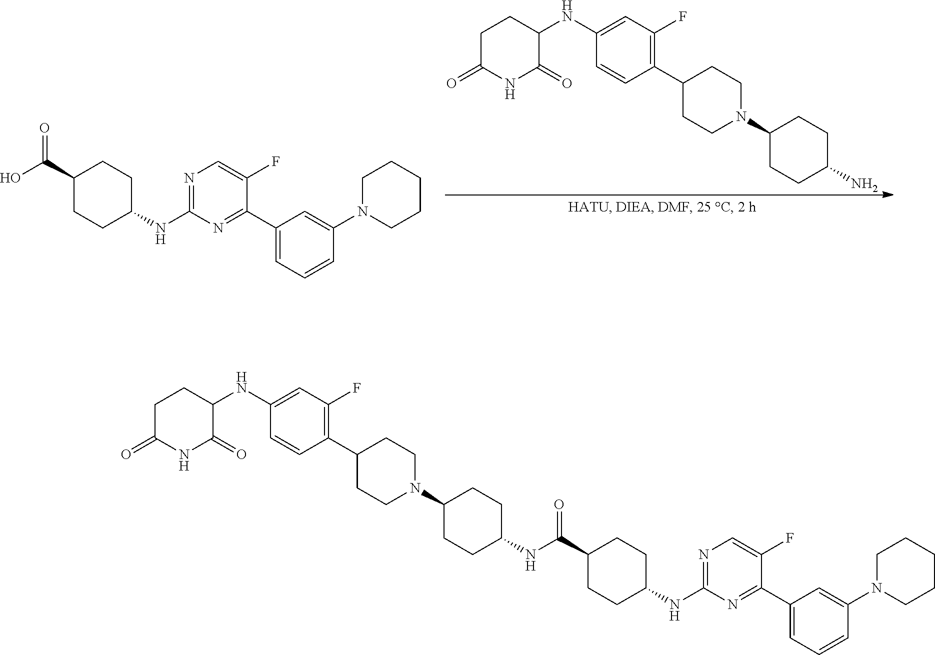 [SMILES: O=C(O)[C@H]1CC[C@H](Nc2ncc(F)c(-c3cccc(N4CCCCC4)c3)n2)CC1 ; N[C@H]1CC[C@H](N2CCC(c3ccc(NC4CCC(=O)NC4=O)cc3F)CC2)CC1 ; O=C1CCC(Nc2ccc(C3CCN([C@H]4CC[C@H](NC(=O)[C@H]5CC[C@H](Nc6ncc(F)c(-c7cccc(N8CCCCC8)c7)n6)CC5)CC4)CC3)c(F)c2)C(=O)N1]

Synthesis of trans-N-(trans-4-(4-(4-((2,6-dioxopiperidin-3-yl)amino)-2-fluorophenyl)piperidin-1-yl)cyclohexyl)-4-((5-fluoro-4-(3-(piperidin-1-yl)phenyl)pyrimidin-2-yl)amino)cyclohexane-1-carboxamide (Compound 515). To a solution of trans-4-[[5-fluoro-4-[3-(1-piperidyl)phenyl]pyrimidin-2-yl]amino]cyclohexanecarboxylic acid (115 mg, 290 μmol, 0.600 eq) in DMF (5 mL) was added trans-3-((4-(1-(4-aminocyclohexyl)piperidin-4-yl)-3-fluorophenyl)amino)piperidine-2,6-dione (250 mg, 484 μmol, 1.00 eq, TFA), DIEA (187 mg, 1.45 mmol, 252 μL, 3.00 eq) and HATU (184 mg, 484 μmol, 1.00 eq) at 25 °C. The mixture was stirred at 25 °C for 2 h. LC-MS showed the desired mass was detected. The reaction mixture was concentrated under reduced pressure. The residue was purified by prep-HPLC (column: Phenomenex Gemini-NX 80*40 mm*3 um; mobile phase: [H₂O (10 mM NH₄HCO₃)-ACN]; gradient: 45%-75% B over 8.0 min) to give the title compound (Compound 515) (41.7 mg, 50.6 μmol, 10.4% yield, 95.1% purity) as a pale yellow solid. LCMS (ES⁺): m/z 783.4 [M+H]⁺.

### Example 585. Synthesis of trans-(1r,4R)—N-((1r,4R)-4-(4-(4-((2,6-dioxopiperidin-3-yl)amino)-2-fluorophenyl)piperazin-1-yl)cyclohexyl)-4-((5-fluoro-4-(3-(piperidin-1-yl)phenyl)pyrimidin-2-yl)amino)cyclohexane-1-carboxamide (Compound 527)

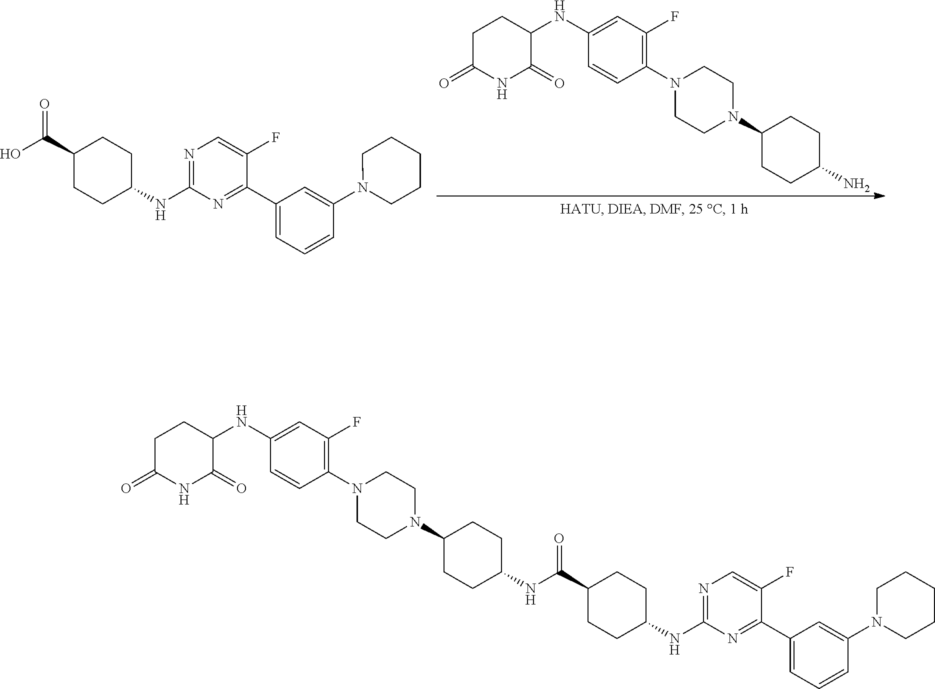 [SMILES: O=C(O)[C@H]1CC[C@H](Nc2ncc(F)c(-c3cccc(N4CCCCC4)c3)n2)CC1 ; N[C@H]1CC[C@H](N2CCN(c3ccc(NC4CCC(=O)NC4=O)cc3F)CC2)CC1 ; O=C1CCC(Nc2ccc(N3CCN([C@H]4CC[C@H](NC(=O)[C@H]5CC[C@H](Nc6ncc(F)c(-c7cccc(N8CCCCC8)c7)n6)CC5)CC4)CC3)c(F)c2)C(=O)N1]

Synthesis of trans-(1r,4R)—N-((1r,4R)-4-(4-(4-((2,6-dioxopiperidin-3-yl)amino)-2-fluorophenyl)piperazin-1-yl)cyclohexyl)-4-((5-fluoro-4-(3-(piperidin-1-yl)phenyl)pyrimidin-2-yl)amino)cyclohexane-1-carboxamide (Compound 527). To a solution of trans-4-[[5-fluoro-4-[3-(1-piperidyl)phenyl]pyrimidin-2-yl]amino]cyclohexanecarboxylic acid (148 mg, 372 mol, 0.500 eq) in DMF (4 mL) were added HATU (99.0 mg, 260 μmol, 0.350 eq), DIEA (480 mg, 3.72 mmol, 648 μL, 5.00 eq) and trans-3-((4-(4-(4-aminocyclohexyl)piperazin-1-yl)-3-fluorophenyl)amino)piperidine-2,6-dione (300 mg, 744 μmol, 1.00 eq) at 25 °C. The mixture was stirred at 25 °C for 1 h. LCMS showed trans-4-[[5-fluoro-4-[3-(1-piperidyl)phenyl]pyrimidin-2-yl]amino]cyclohexanecarboxylic acid was consumed completely and the desired mass was detected. The reaction mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 45%-75% B over 8.0 min) to give the title compound (Compound 527) (50.9 mg, 64.7 μmol, 8.71% yield, 99.7% purity) as a yellow solid. LCMS (ES⁺): m/z 784.5 [M+H]⁺.

### Example 586. Synthesis of trans-N-(1′-(4-((2,6-dioxopiperidin-3-yl)amino)-2-fluorophenyl)-[1,4′-bipiperidin]-4-yl)-4-((5-fluoro-4-(3-(piperidin-1-yl)phenyl)pyrimidin-2-yl)amino)cyclohexane-1-carboxamide (Compound 531)

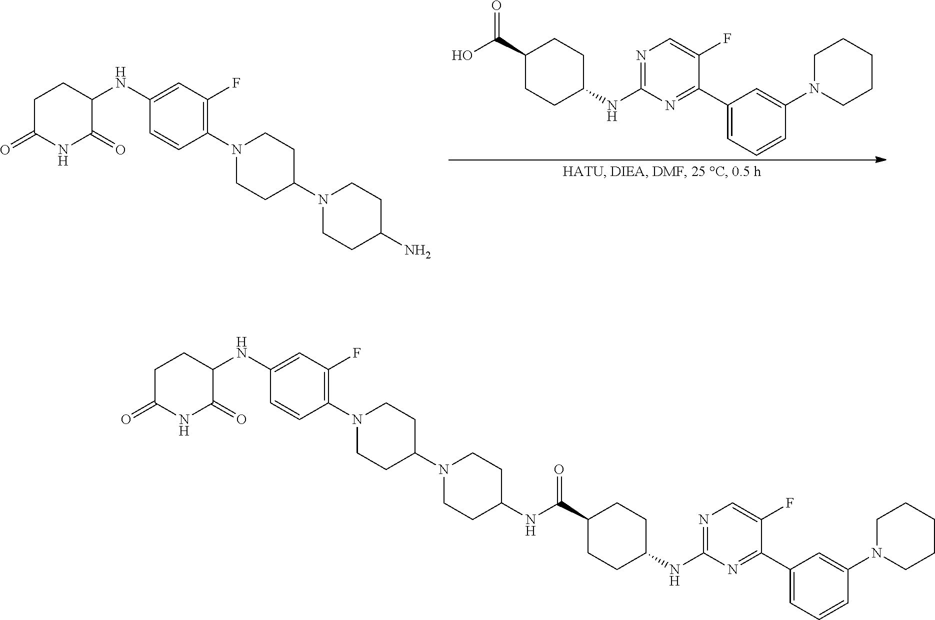 [SMILES: NC1CCN(C2CCN(c3ccc(NC4CCC(=O)NC4=O)cc3F)CC2)CC1 ; O=C(O)[C@H]1CC[C@H](Nc2ncc(F)c(-c3cccc(N4CCCCC4)c3)n2)CC1 ; O=C1CCC(Nc2ccc(N3CCC(N4CCC(NC(=O)[C@H]5CC[C@H](Nc6ncc(F)c(-c7cccc(N8CCCCC8)c7)n6)CC5)CC4)CC3)c(F)c2)C(=O)N1]

Synthesis of trans-N-(1′-(4-((2,6-dioxopiperidin-3-yl)amino)-2-fluorophenyl)-[1,4′-bipiperidin]-4-yl)-4-((5-fluoro-4-(3-(piperidin-1-yl)phenyl)pyrimidin-2-yl)amino)cyclohexane-1-carboxamide (Compound 531). To a solution of trans-4-((5-fluoro-4-(3-(piperidin-1-yl)phenyl)pyrimidin-2-yl)amino)cyclohexane-1-carboxylic acid (83.0 mg, 208 μmol, 0.350 eq) in DMF (3 mL) were added HATU (79.2 mg, 208 μmol, 0.350 eq), DIEA (385 mg, 2.98 mmol, 518 μL, 5.00 eq) and 3-((4-(4-amino-[1,4′-bipiperidin]-1′-yl)-3-fluorophenyl)amino)piperidine-2,6-dione (308 mg, 595 μmol, 1.00 eq, TFA) at 25 °C. The mixture was stirred at 25 °C for 0.5 h. LC-MS showed 3-((4-(4-amino-[1,4′-bipiperidin]-1′-yl)-3-fluorophenyl)amino)piperidine-2,6-dione was consumed completely and the desired mass was detected. The reaction mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 40%-80% B over 8.0 min) to give the title compound (Compound 531) (41.8 mg, 51.8 μmol, 8.70% yield, 97.1% purity) as a pale yellow solid. LCMS (ES⁺): m/z 784.5 [M+H]⁺

### Example 587. Synthesis of trans-3-[3-fluoro-4-[4-[[1-[4-[[5-fluoro-4-[3-(1-piperidyl)phenyl]pyrimidin-2-yl]amino]cyclohexanecarbonyl]-4-hydroxy-4-piperidyl]methyl]piperazin-1-yl]anilino]piperidine-2,6-dione (Compound 493)

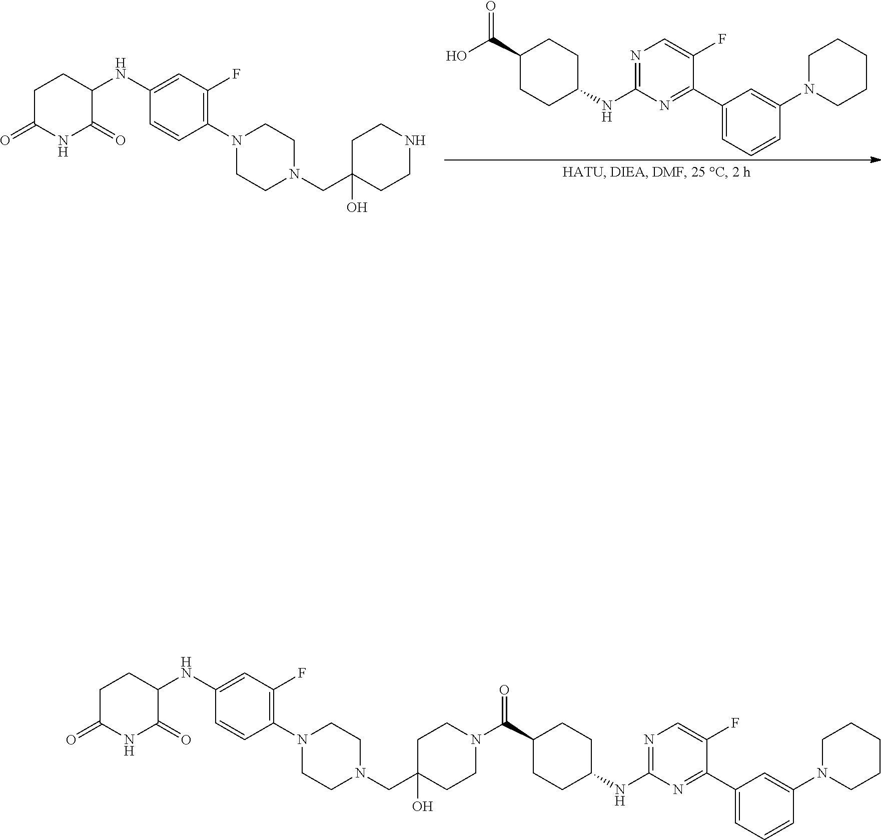 [SMILES: O=C1CCC(Nc2ccc(N3CCN(CC4(O)CCNCC4)CC3)c(F)c2)C(=O)N1 ; O=C(O)[C@H]1CC[C@H](Nc2ncc(F)c(-c3cccc(N4CCCCC4)c3)n2)CC1 ; O=C1CCC(Nc2ccc(N3CCN(CC4(O)CCN(C(=O)[C@H]5CC[C@H](Nc6ncc(F)c(-c7cccc(N8CCCCC8)c7)n6)CC5)CC4)CC3)c(F)c2)C(=O)N1]

Synthesis of trans-3-[3-fluoro-4-[4-[[1-[4-[[5-fluoro-4-[3-(1-piperidyl)phenyl]pyrimidin-2-yl]amino]cyclohexanecarbonyl]-4-hydroxy-4-piperidyl]methyl]piperazin-1-yl]anilino]piperidine-2,6-dione (Compound 493). To a solution of 3-((3-fluoro-4-(4-((4-hydroxypiperidin-4-yl)methyl)piperazin-1-yl)phenyl)amino)piperidine-2,6-dione (300 mg, 562 μmol, 1.00 eq, TFA) and trans-4-[[5-fluoro-4-[3-(1-piperidyl)phenyl]pyrimidin-2-yl]amino]cyclohexanecarboxylic acid (224 mg, 562 μmol, 1.00 eq) in DMF (3 mL) was added DIEA (218 mg, 1.69 mmol, 293 μL, 3.00 eq) and HATU (320 mg, 843 μmol, 1.50 eq) at 25 °C. The mixture was stirred at 25 °C for 2 h. LC-MS showed 3-((3-fluoro-4-(4-((4-hydroxypiperidin-4-yl)methyl)piperazin-1-yl)phenyl)amino)piperidine-2,6-dione was consumed completely and the desired mass was detected. The reaction mixture was concentrated under reduced pressure. The residue was purified by prep-HPLC (column: Phenomenex luna C18 100*40 mm*3 um; mobile phase: [H₂O (0.2% FA)-ACN]; gradient: 5%-40% B over 8.0 min). The residue was purified by prep-HPLC (column: Phenomenex luna C18 100*40 mm*5 um; mobile phase: [H₂O (0.04% HCl)-ACN]; gradient: 5%-40% B over 8.0 min) to give the title compound (Compound 493) (61.0 mg, 73.7 μmol, 13.1% yield, 96.7% purity) as a white solid. LCMS (ES⁺): m/z 800.5 [M+H]⁺.

### Example 588. Synthesis of trans-3-((3-fluoro-4-(1-((1-(4-((5-fluoro-4-(3-(piperidin-1-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 514)

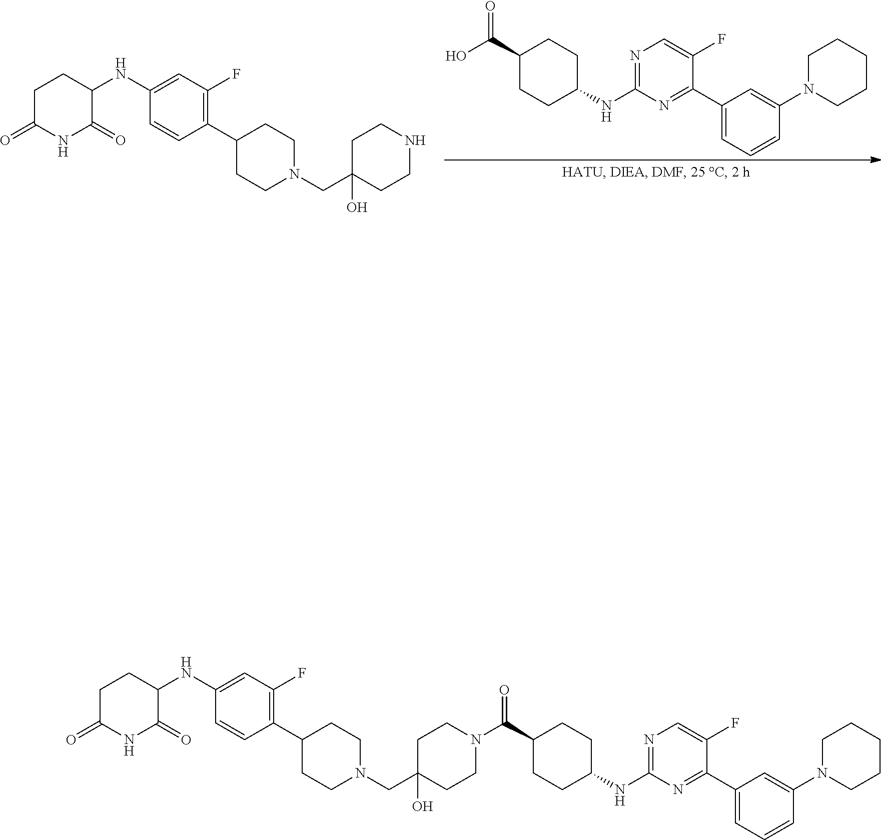 [SMILES: O=C1CCC(Nc2ccc(C3CCN(CC4(O)CCNCC4)CC3)c(F)c2)C(=O)N1 ; O=C(O)[C@H]1CC[C@H](Nc2ncc(F)c(-c3cccc(N4CCCCC4)c3)n2)CC1 ; O=C1CCC(Nc2ccc(C3CCN(CC4(O)CCN(C(=O)[C@H]5CC[C@H](Nc6ncc(F)c(-c7cccc(N8CCCCC8)c7)n6)CC5)CC4)CC3)c(F)c2)C(=O)N1]

Synthesis of trans-3-((3-fluoro-4-(1-((1-(4-((5-fluoro-4-(3-(piperidin-1-yl)phenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 514). To a solution of 3-((3-fluoro-4-(1-((4-hydroxypiperidin-4-yl)methyl)piperidin-4-yl)phenyl)amino)piperidine-2,6-dione (250 mg, 469 μmol, 1.00 eq, TFA) in DMF (5 mL) was added trans-4-[[5-fluoro-4-[3-(1-piperidyl)phenyl]pyrimidin-2-yl]amino]cyclohexanecarboxylic acid (112 mg, 281 μmol, 0.600 eq), DIEA (60.6 mg, 469 μmol, 81.7 μL, 1.00 eq) and HATU (178 mg, 469 μmol, 1.00 eq) at 25 °C. The mixture was stirred at 25 °C for 2 h. LC-MS showed the desired mass was detected. The reaction mixture was concentrated under reduced pressure. The residue was purified by prep-HPLC (column: Waters Xbridge Prep OBD C18 150*40 mm*10 um; mobile phase: [H₂O (10 mM NH₄HCO₃)-ACN]; gradient: 55%-85% B over 8.0 min) to give the title compound (Compound 514) (41.5 mg, 50.6 μmol, 10.7% yield, 97.5% purity) as a yellow solid. LCMS (ES⁺): m/z 799.4 [M+H]⁺.

### Example 589. Synthesis of trans-3-((3-fluoro-4-(4-((1-(4-((5-fluoro-4-(3-(piperidin-1-yl)phenyl)pyrimidin-2-yl)amino)cyclohexane-1-carbonyl)piperidin-4-yl)methyl)piperazin-1-yl)phenyl)amino)piperidine-2,6-dione (Compound 513)

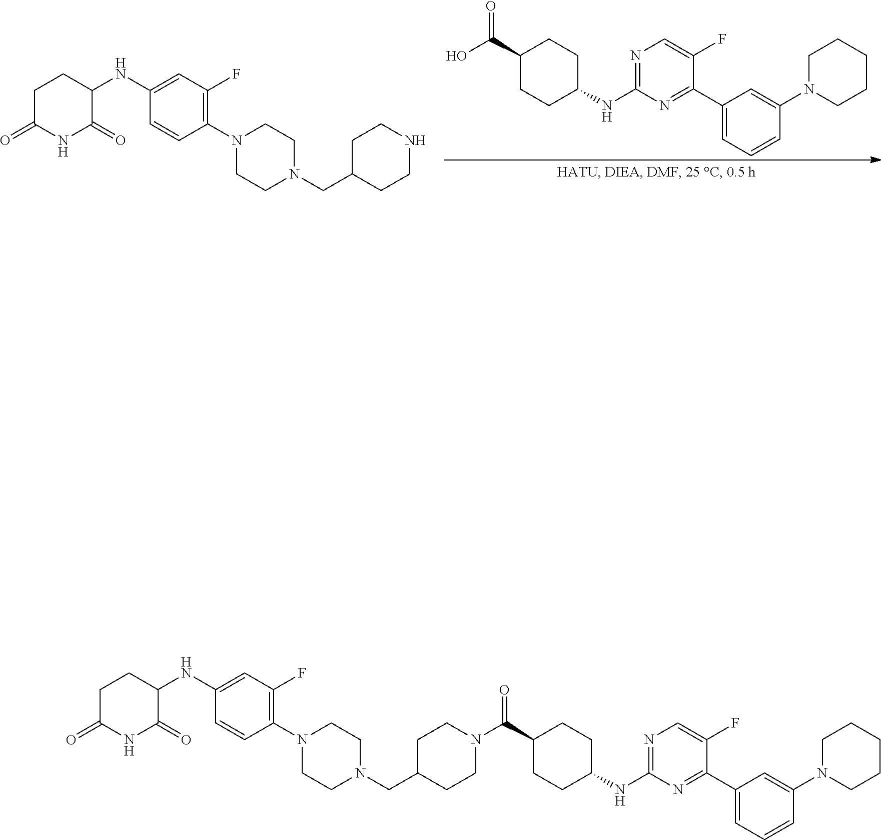 [SMILES: O=C1CCC(Nc2ccc(N3CCN(CC4CCNCC4)CC3)c(F)c2)C(=O)N1 ; O=C(O)[C@H]1CC[C@H](Nc2ncc(F)c(-c3cccc(N4CCCCC4)c3)n2)CC1 ; O=C1CCC(Nc2ccc(N3CCN(CC4CCN(C(=O)[C@H]5CC[C@H](Nc6ncc(F)c(-c7cccc(N8CCCCC8)c7)n6)CC5)CC4)CC3)c(F)c2)C(=O)N1]

Synthesis of trans-3-((3-fluoro-4-(4-((1-(4-((5-fluoro-4-(3-(piperidin-1-yl)phenyl)pyrimidin-2-yl)amino)cyclohexane-1-carbonyl)piperidin-4-yl)methyl)piperazin-1-yl)phenyl)amino)piperidine-2,6-dione (Compound 513). To a solution of 3-((3-fluoro-4-(4-(piperidin-4-ylmethyl)piperazin-1-yl)phenyl)amino)piperidine-2,6-dione (240 mg, 463 μmol, 1.00 eq, TFA) in DMF (3 mL) was added trans-4-((5-fluoro-4-(3-(piperidin-1-yl)phenyl)pyrimidin-2-yl)amino)cyclohexane-1-carboxylic acid (184 mg, 463 μmol, 1.00 eq), HATU (176 mg, 463 μmol, 1.00 eq) and DIEA (179.18 mg, 1.389 μmol, 3.00 eq). The mixture was stirred at 25 °C for 0.5 h. LC-MS showed the desired mass was detected. The reaction mixture was quenched by addition H₂O 3 mL, and then extracted with EtOAc (3 mL*3). The combined organic layers were dried over Na₂SO₄, filtered and concentrated under reduced pressure. The residue was purified by prep-HPLC (column: Phenomenex luna C18 100*40 mm*3 um; mobile phase: [H₂O (0.2% FA)-ACN]; gradient: 10%-35% B over 8.0 min) to give the title compound (Compound 513) (114 mg, 142 μmol, 30.6% yield, 97.6% purity) as a yellow solid. LCMS (ES⁺): m/z 784.5 [M+H]⁺.

### Example 590. Synthesis of trans-3-((3-fluoro-4-(1-((1-(trans-4-((5-fluoro-4-(3-(piperidin-1-yl)phenyl)pyrimidin-2-yl)amino)cyclohexane-1-carbonyl)piperidin-4-yl)methyl)piperidin-4-yl)phenyl)amino)piperidine-2,6-dione (Compound 477)

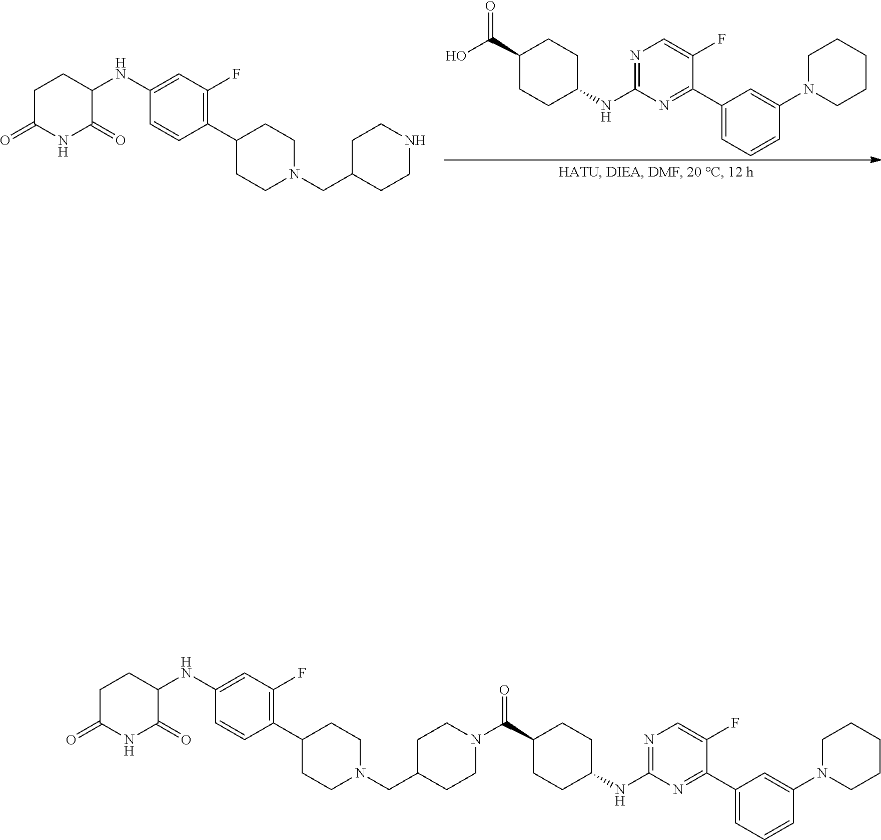 [SMILES: O=C1CCC(Nc2ccc(C3CCN(CC4CCNCC4)CC3)c(F)c2)C(=O)N1 ; O=C(O)[C@H]1CC[C@H](Nc2ncc(F)c(-c3cccc(N4CCCCC4)c3)n2)CC1 ; O=C1CCC(Nc2ccc(C3CCN(CC4CCN(C(=O)[C@H]5CC[C@H](Nc6ncc(F)c(-c7cccc(N8CCCCC8)c7)n6)CC5)CC4)CC3)c(F)c2)C(=O)N1]

Synthesis of trans-3-((3-fluoro-4-(1-((1-(trans-4-((5-fluoro-4-(3-(piperidin-1-yl)phenyl)pyrimidin-2-yl)amino)cyclohexane-1-carbonyl)piperidin-4-yl)methyl)piperidin-4-yl)phenyl)amino)piperidine-2,6-dione (Compound 477). To a solution of 3-((3-fluoro-4-(1-(piperidin-4-ylmethyl)piperidin-4-yl)phenyl)amino)piperidine-2,6-dione (300 mg, 745 μmol, 1.00 eq) in DMF (5 mL) was added trans-4-[[5-fluoro-4-[3-(1-piperidyl)phenyl]pyrimidin-2-yl]amino]cyclohexanecarboxylic acid (148 mg, 372 μmol, 0.500 eq), DIEA (192 mg, 1.49 mmol, 259 μL, 2.00 eq) and HATU (566 mg, 1.49 mmol, 2.00 eq) at 20 °C. The mixture was stirred at 20 °C for 12 h. LC-MS showed the desired mass was detected. The reaction mixture was concentrated under high vacuum. The residue was purified by prep-HPLC (Waters Xbridge BEH C18 100*30 mm*10 um; mobile phase: [H₂O (10 mM NH₄HCO₃)-ACN]; gradient: 66%-96% B over 8.0 min) to give the title compound (Compound 477) (15.9 mg, 19.8 μmol, 2.67% yield, 97.9% purity) as a pale yellow solid. LCMS (ES⁺): m/z 782.8 [M+H]⁺.

### Example 591. Synthesis of trans-3-((3-fluoro-4-(4-((4-fluoro-1-(4-((5-fluoro-4-(3-(piperidin-1-yl)phenyl)pyrimidin-2-yl)amino)cyclohexane-1-carbonyl)piperidin-4-yl)methyl)piperazin-1-yl)phenyl)amino)piperidine-2,6-dione (Compound 491)

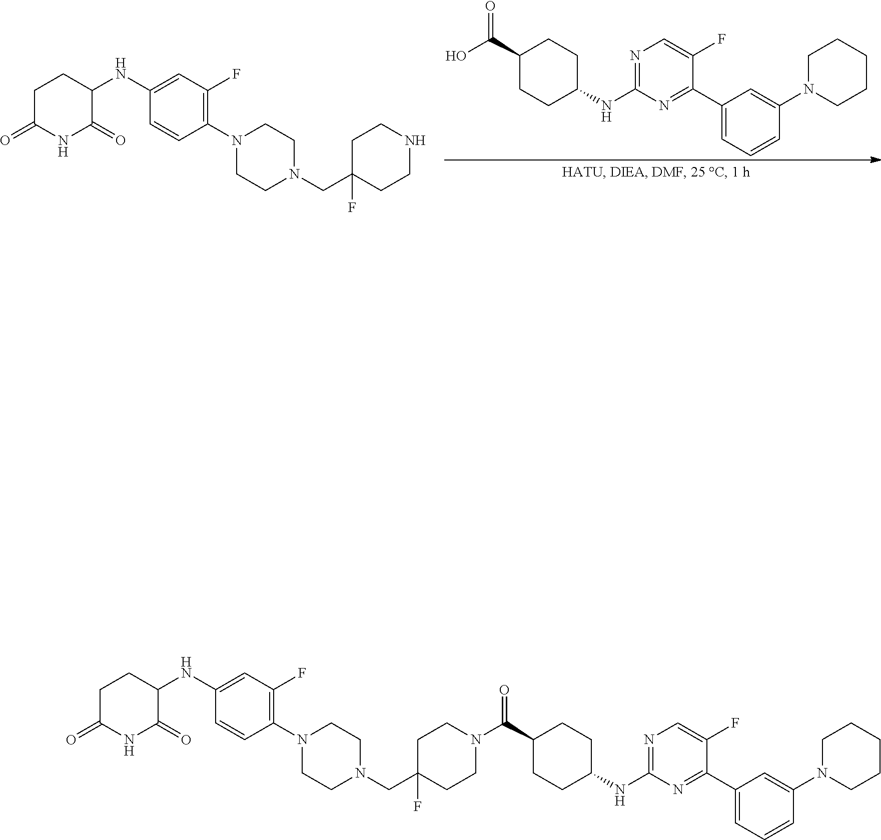 [SMILES: O=C1CCC(Nc2ccc(N3CCN(CC4(F)CCNCC4)CC3)c(F)c2)C(=O)N1 ; O=C(O)[C@H]1CC[C@H](Nc2ncc(F)c(-c3cccc(N4CCCCC4)c3)n2)CC1 ; O=C1CCC(Nc2ccc(N3CCN(CC4(F)CCN(C(=O)[C@H]5CC[C@H](Nc6ncc(F)c(-c7cccc(N8CCCCC8)c7)n6)CC5)CC4)CC3)c(F)c2)C(=O)N1]

Synthesis of trans-3-((3-fluoro-4-(4-((4-fluoro-1-(4-((5-fluoro-4-(3-(piperidin-1-yl)phenyl)pyrimidin-2-yl)amino)cyclohexane-1-carbonyl)piperidin-4-yl)methyl)piperazin-1-yl)phenyl)amino)piperidine-2,6-dione (Compound 491). To a solution of 3-((3-fluoro-4-(4-((4-fluoropiperidin-4-yl)methyl)piperazin-1-yl)phenyl)amino)piperidine-2,6-dione (300 mg, 560 μmol, 1.00 eq, TFA) in DMF (1.5 mL) was added DIEA (217 mg, 1.68 mmol, 292 μL, 3.00 eq) until pH-7. To a solution of trans-4-((5-fluoro-4-(3-(piperidin-1-yl)phenyl)pyrimidin-2-yl)amino)cyclohexane-1-carboxylic acid (223 mg, 560 μmol, 1.00 eq) in DMF (1.5 mL) was added DIEA (217 mg, 1.68 mmol, 292 μL, 3.00 eq) until pH-7. Then HATU (319 mg, 840 μmol, 1.50 eq) was added to the mixture of the two solutions. The mixture was stirred at 25 °C for 1 h. LC-MS showed the desired mass was detected. The reaction mixture was filtered to give a residue. The residue was purified by prep-HPLC (column: Phenomenex Luna C18 100*30 mm*3 um; mobile phase: [H₂O (0.2% FA)-ACN]; gradient: 5%-35% B over 8.0 min) to give the title compound (Compound 491) (20.8 mg, 25.2 μmol, 4.51% yield, 97.3% purity) as a gray solid. LCMS (ES⁺): m/z 802.2 [M+H]⁺.

### Example 592. Synthesis of 3-((4-(1-((1-(trans-4-((4-([1,1′-biphenyl]-3-yl)-5-fluoropyrimidin-2-yl)amino)cyclohexane-1-carbonyl)piperidin-4-yl)methyl)piperidin-4-yl)-3-fluorophenyl)amino)piperidine-2,6-dione (Compound 466)

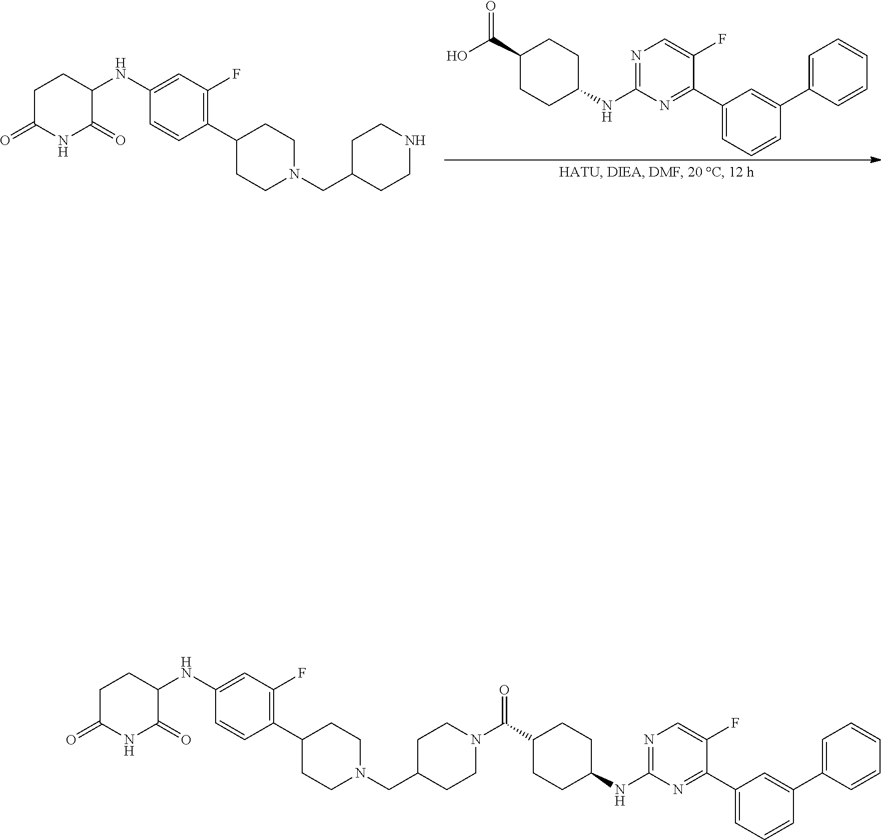

Synthesis of 3-((4-(1-((1-(trans-4-((4-([1,1′-biphenyl]-3-yl)-5-fluoropyrimidin-2-yl)amino)cyclohexane-1-carbonyl)piperidin-4-yl)methyl)piperidin-4-yl)-3-fluorophenyl)amino)piperidine-2,6-dione (Compound 466). To a solution of 3-((3-fluoro-4-(1-(piperidin-4-ylmethyl)piperidin-4-yl)phenyl)amino)piperidine-2,6-dione (200 mg, 387 μmol, 1.00 eq, TFA) in DMF (5 mL) was added trans-4-((4-([1,1′-biphenyl]-3-yl)-5-fluoropyrimidin-2-yl)amino)cyclohexane-1-carboxylic acid (151 mg, 387 μmol, 1.00 eq) HATU (294 mg, 774 μmol, 2.00 eq) and DIEA (100 mg, 774 μmol, 134 μL, 2.00 eq) at 20 °C. The mixture was stirred at 20 °C for 12 h. LC-MS showed the desired mass was detected. The reaction mixture was filtered and the filtrate concentrated under high vacuum. The residue was purified by prep-HPLC (3_Phenomenex Luna C18 75*30 mm*3 um; mobile phase: [H₂O (0.2% FA)-ACN]; gradient: 25%-55% B over 8.0 min) to give the title compound (Compound 466) (45.9 mg, 58.1 μmol, 15.0% yield, 100% purity, 0.3 FA) as a pale yellow solid. LCMS (ES⁺): m/z 776.4 [M+H]⁺.

### Example 593. Synthesis of trans-3-((4-(4-((1-(4-((4-([1,1′-biphenyl]-3-yl)-5-fluoropyrimidin-2-yl)amino)cyclohexane-1-carbonyl)piperidin-4-yl)methyl)piperazin-1-yl)-3-fluorophenyl)amino)piperidine-2,6-dione (Compound 485)

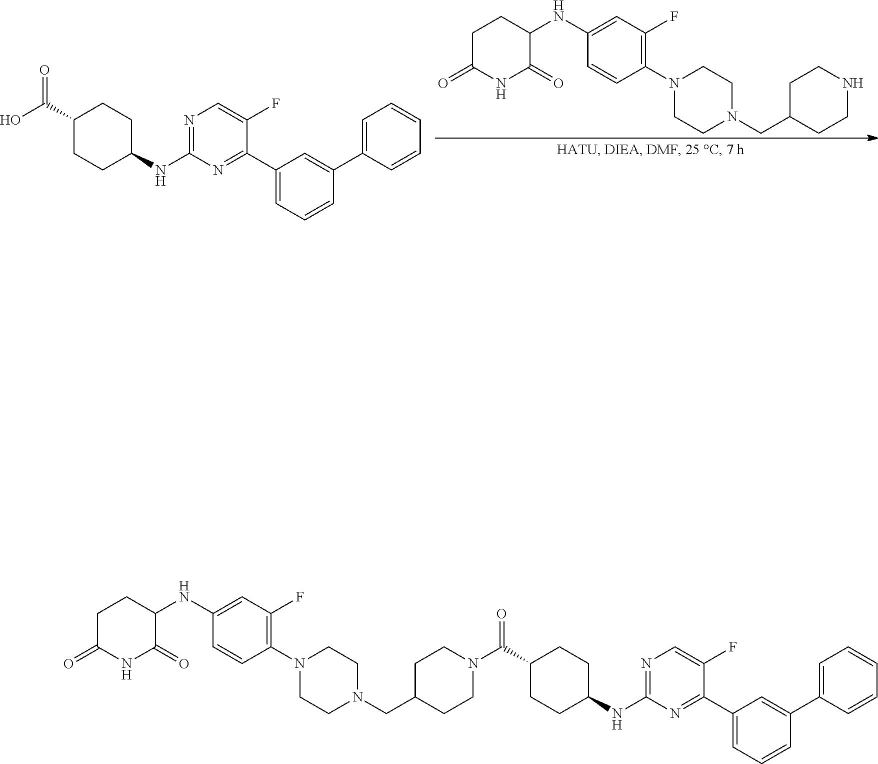

Synthesis of trans-3-((4-(4-((1-(4-((4-([1,1′-biphenyl]-3-yl)-5-fluoropyrimidin-2-yl)amino)cyclohexane-1-carbonyl)piperidin-4-yl)methyl)piperazin-1-yl)-3-fluorophenyl)amino)piperidine-2,6-dione (Compound 485). To a solution of trans-4-((4-([1,1′-biphenyl]-3-yl)-5-fluoropyrimidin-2-yl)amino)cyclohexane-1-carboxylic acid (114 mg, 290 μmol, 0.700 eq) in DMF (4 mL) were added DIEA (268 mg, 2.08 mmol, 363 μL, 5.00 eq) and HATU (63.1 mg, 166 μmol, 0.400 eq) at 25 °C. Then to the mixture was added 3-((3-fluoro-4-(4-(piperidin-4-ylmethyl)piperazin-1-yl)phenyl)amino)piperidine-2,6-dione (215 mg, 415 μmol, 1.00 eq, TFA) at 25 °C. The mixture was stirred at 25 °C for 7 h. LCMS showed 4-((4-([1,1′-biphenyl]-3-yl)-5-fluoropyrimidin-2-yl)amino)cyclohexane-1-carboxylic acid was consumed completely and the desired mass was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 50%-80% B over 8.0 min) to give the title compound (Compound 485) (83.7 mg, 97.8 μmol, 23.6% yield, 90.8% purity) as a pale yellow solid. LCMS (ES⁺): m/z 777.4 [M+H]⁺

### Example 594. Synthesis of trans-3-[3-fluoro-4-[4-[[4-fluoro-1-[4-[[5-fluoro-4-(3-phenylphenyl) pyrimidin-2-yl]amino]cyclohexanecarbonyl]-4-piperidyl]methyl]piperazin-1-yl]anilino]piperidine-2,6-dione (Compound 484)

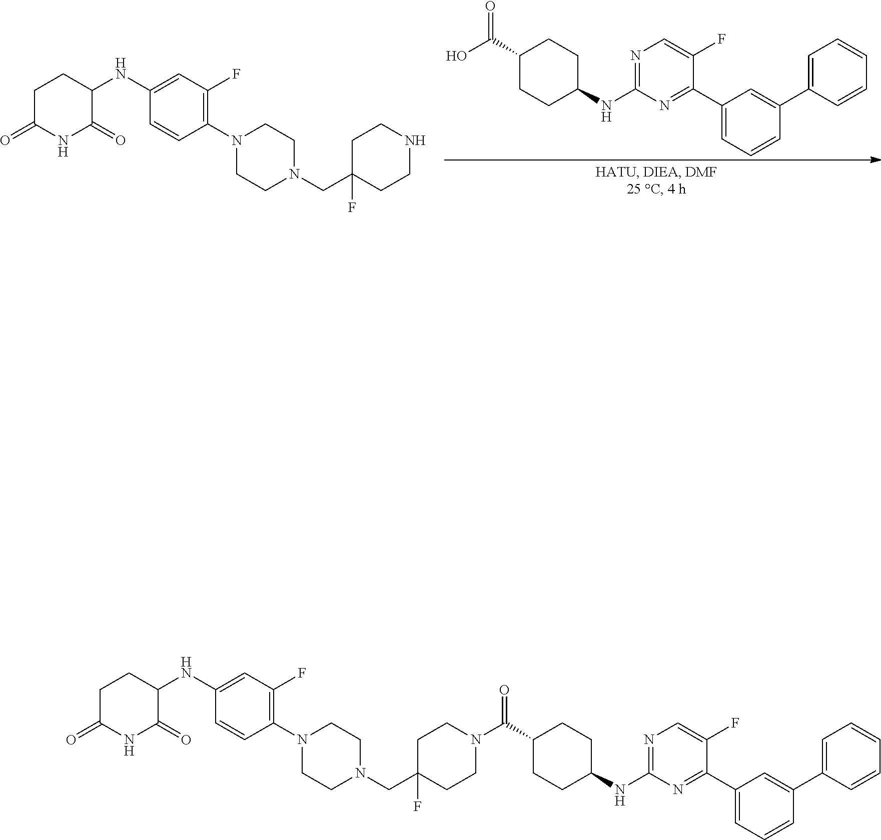 [SMILES: O=C1CCC(Nc2ccc(N3CCN(CC4(F)CCNCC4)CC3)c(F)c2)C(=O)N1 ; O=C(O)[C@H]1CC[C@H](Nc2ncc(F)c(-c3cccc(-c4ccccc4)c3)n2)CC1 ; O=C1CCC(Nc2ccc(N3CCN(CC4(F)CCN(C(=O)[C@H]5CC[C@H](Nc6ncc(F)c(-c7cccc(-c8ccccc8)c7)n6)CC5)CC4)CC3)c(F)c2)C(=O)N1]

Synthesis of trans-3-[3-fluoro-4-[4-[[4-fluoro-1-[4-[[5-fluoro-4-(3-phenylphenyl) pyrimidin-2-yl]amino]cyclohexanecarbonyl]-4-piperidyl]methyl]piperazin-1-yl]anilino]piperidine-2,6-dione (Compound 484). To a solution of trans-4-[[5-fluoro-4-(3-phenylphenyl) pyrimidin-2-yl]amino]cyclohexanecarboxylic acid (100 mg, 256 μmol, 0.700 eq) in DMF (3 mL) were added HATU (55.5 mg, 146 μmol, 0.400 eq) and DIEA (236 mg, 1.83 mmol, 318 μL, 5.00 eq) at 25 °C. After addition, 3-[3-fluoro-4-[4-[(4-fluoro-4-piperidyl) methyl]piperazin-1-yl]anilino]piperidine-2,6-dione (195 mg, 365 μmol, 1.00 eq, TFA) was added to the mixture at 25 °C. The resulting mixture was stirred at 25 °C for 4 h. LCMS showed 3-((3-fluoro-4-(4-((4-fluoropiperidin-4-yl)methyl)piperazin-1-yl)phenyl)amino)piperidine-2,6-dione was consumed and the desired mass was detected. The mixture was concentrated under reduced pressure to give a residue. The residue was purified by prep-HPLC (column: Phenomenex Gemini-NX 80*40 mm*3 um; mobile phase: [H₂O (10 mM NH₄HCO₃)-ACN]; gradient: 45%-75% B over 8.0 min) to give the title compound (Compound 484) (76.7 mg, 96.3 μmol, 26.4% yield, 99.8% purity) as a white solid. LCMS (ES⁺): m/s 795.4 [M+H]⁺

### Example 595. Synthesis of 3-((4-(1-((1-(trans-4-((4-([1,1′-biphenyl]-3-yl)-5-fluoropyrimidin-2-yl)amino)cyclohexane-1-carbonyl)-4-hydroxypiperidin-4-yl)methyl)piperidin-4-yl)-3-fluorophenyl)amino)piperidine-2,6-dione (Compound 464)

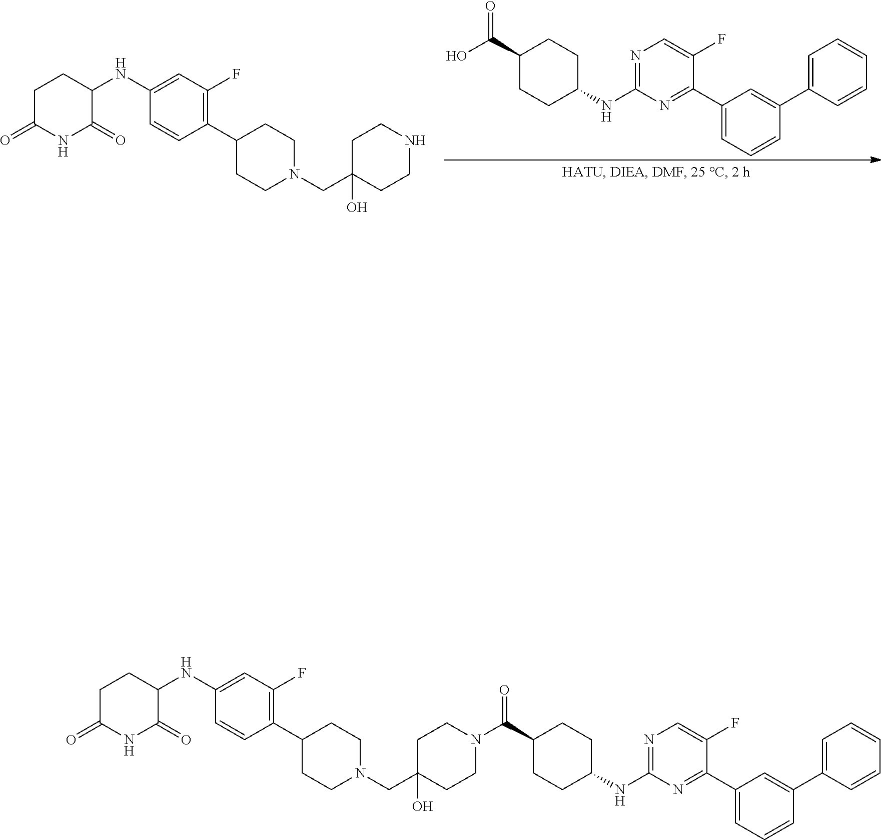 [SMILES: O=C1CCC(Nc2ccc(C3CCN(CC4(O)CCNCC4)CC3)c(F)c2)C(=O)N1 ; O=C(O)[C@H]1CC[C@H](Nc2ncc(F)c(-c3cccc(-c4ccccc4)c3)n2)CC1 ; O=C1CCC(Nc2ccc(C3CCN(CC4(O)CCN(C(=O)[C@H]5CC[C@H](Nc6ncc(F)c(-c7cccc(-c8ccccc8)c7)n6)CC5)CC4)CC3)c(F)c2)C(=O)N1]

Synthesis of 3-((4-(1-((1-(trans-4-((4-([1,1′-biphenyl]-3-yl)-5-fluoropyrimidin-2-yl)amino)cyclohexane-1-carbonyl)-4-hydroxypiperidin-4-yl)methyl)piperidin-4-yl)-3-fluorophenyl)amino)piperidine-2,6-dione (Compound 464). To a solution of 3-[3-fluoro-4-[1-[(4-hydroxy-4-piperidyl)methyl]-4-piperidyl]anilino]piperidine-2,6-dione (200 mg, 375 mol, 1.00 eq, TFA) in DMF (2 mL) was added HATU (285 mg, 751 μmol, 2.00 eq), DIEA (97.1 mg, 751 μmol, 131 μL, 2.00 eq) and trans-4-((4-([1,1′-biphenyl]-3-yl)-5-fluoropyrimidin-2-yl)amino)cyclohexane-1-carboxylic acid (147 mg, 375 μmol, 1.00 eq) at 25 °C. The mixture was stirred at 25 °C for 2 h. LC-MS showed the desired mass was detected. The reaction mixture was quenched by addition H₂O 3 mL, and then extracted with EtOAc (3 mL*3). The combined organic layers were dried over Na₂SO₄, filtered and concentrated under reduced pressure. The residue was purified by prep-HPLC (column: Phenomenex luna C18 100*40 mm*3 um; mobile phase: [H₂O (0.2% FA)-ACN]; gradient: 25%-60% B over 8.0 min) to give the title compound (Compound 464) (213 mg, 269 μmol, 71.6% yield, 100% purity) as a pale yellow solid. LCMS (ES⁺): m/z 792.5 [M+H]⁺.

### Example 596. Synthesis of trans-3-[3-fluoro-4-[4-[[1-[4-[[5-fluoro-4-(3-phenylphenyl)pyrimidin-2-yl]amino]cyclohexanecarbonyl]-4-hydroxy-4-piperidyl]methyl]piperazin-1-yl]anilino]piperidine-2,6-dione (Compound 463)

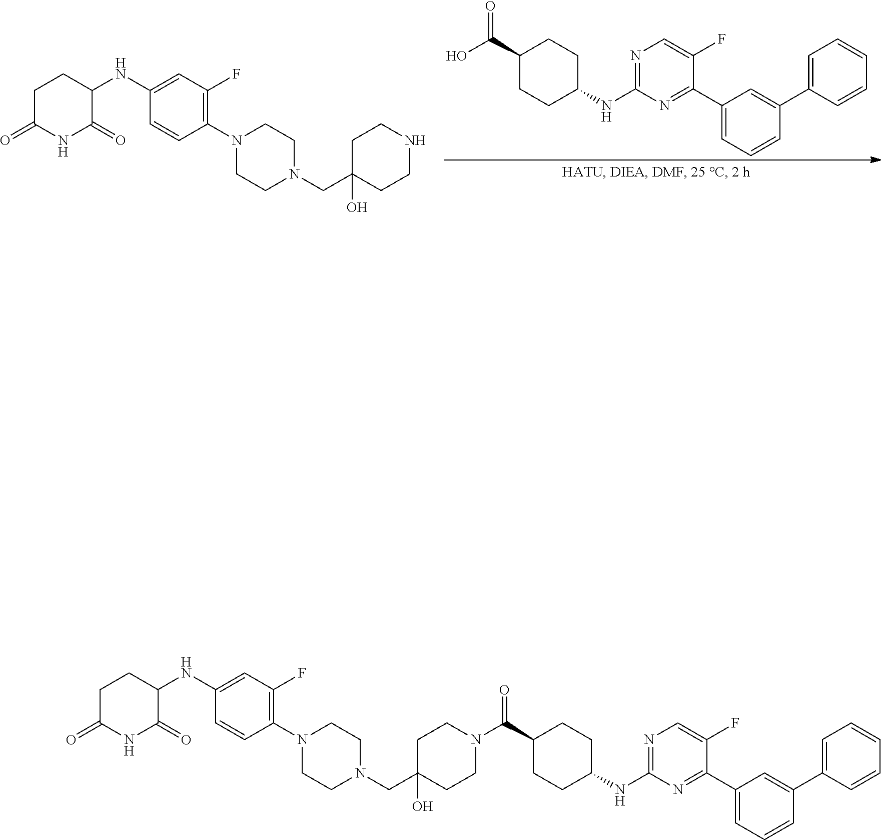 [SMILES: O=C1CCC(Nc2ccc(N3CCN(CC4(O)CCNCC4)CC3)c(F)c2)C(=O)N1 ; O=C(O)[C@H]1CC[C@H](Nc2ncc(F)c(-c3cccc(-c4ccccc4)c3)n2)CC1 ; O=C1CCC(Nc2ccc(N3CCN(CC4(O)CCN(C(=O)[C@H]5CC[C@H](Nc6ncc(F)c(-c7cccc(-c8ccccc8)c7)n6)CC5)CC4)CC3)c(F)c2)C(=O)N1]

Synthesis of trans-3-[3-fluoro-4-[4-[[1-[4-[[5-fluoro-4-(3-phenylphenyl)pyrimidin-2-yl]amino]cyclohexanecarbonyl]-4-hydroxy-4-piperidyl]methyl]piperazin-1-yl]anilino]piperidine-2,6-dione (Compound 463). To a solution of 3-[3-fluoro-4-[4-[(4-hydroxy-4-piperidyl)methyl]piperazin-1-yl]anilino]piperidine-2,6-dione (200 mg, 374 μmol, 1.00 eq, TFA) and trans-4-[[5-fluoro-4-(3-phenylphenyl)pyrimidin-2-yl]amino]cyclohexanecarboxylic acid (146 mg, 374 μmol, 1.00 eq) in DMF (3 mL) was added HATU (213 mg, 562 μmol, 1.50 eq) and DIEA (145 mg, 1.12 mmol, 195 μL, 3.00 eq) at 25 °C. The mixture was stirred at 25 °C for 2 h. LC-MS showed the desired mass was detected. The reaction mixture was concentrated under reduced pressure. The residue was purified by prep-HPLC (column: Phenomenex Luna C18 100*30 mm*3 um; mobile phase: [H₂O (0.2% FA)-ACN]; gradient: 20%-55% B over 8.0 min) to give the title compound (Compound 463) (47.2 mg, 57.9 μmol, 15.4% yield, 97.2% purity) as a pale yellow solid. LCMS (ES⁺): m/z 793.4 [M+H]⁺.

### Example 597. Synthesis of 3-((3-fluoro-4-(1-((1-(4-((5-fluoro-4-(3-(2-oxopyridin-1 (2H)-yl)phenyl)pyrimidin-2-yl)amino)piperidine-1-carbonyl)piperidin-4-yl)methyl)piperidin-4-yl)phenyl)amino)piperidine-2,6-dione (Compound 465)

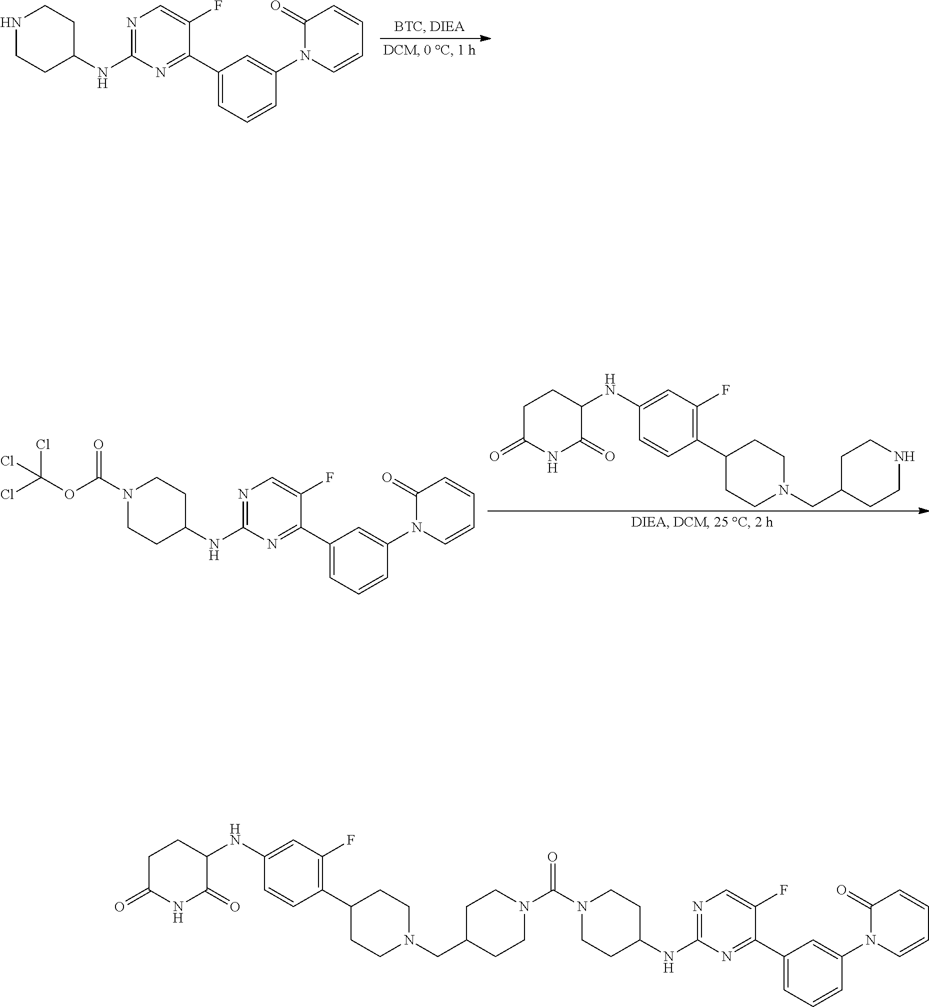

Synthesis of trichloromethyl 4-((5-fluoro-4-(3-(2-oxopyridin-1 (2H)-yl)phenyl)pyrimidin-2-yl)amino)piperidine-1-carboxylate. To a solution of 1-[3-[5-fluoro-2-(4-piperidylamino)pyrimidin-4-yl]phenyl]pyridin-2-one (200 mg, 497 μmol, 1.00 eq, HCl) in DCM (1 mL) was added DIEA (128 mg, 995 μmol, 173 μL, 2.00 eq) and bis(trichloromethyl) carbonate (120 mg, 404 μmol, 0.813). The mixture was stirred at 0 °C for 1 h. TLC (Petroleum ether:Ethyl acetate=3:1, Rf=0.67) indicated one major new spot was detected. Compound (200 mg, crude) in DCM (1 mL) was used to next step directly.

Synthesis of 3-((3-fluoro-4-(1-((1-(4-((5-fluoro-4-(3-(2-oxopyridin-1 (2H)-yl)phenyl)pyrimidin-2-yl)amino)piperidine-1-carbonyl)piperidin-4-yl)methyl)piperidin-4-yl)phenyl)amino)piperidine-2,6-dione (Compound 465). To a solution of trichloromethyl 4-((5-fluoro-4-(3-(2-oxopyridin-1 (2H)-yl)phenyl)pyrimidin-2-yl)amino)piperidine-1-carboxylate (100 mg, 189 μmol, 1.00 eq) in DCM (1 mL) was added DIEA (49.1 mg, 379 μmol, 66.1 μL, 2.00 eq) and 3-[3-fluoro-4-[1-(4-piperidylmethyl)-4-piperidyl]anilino]piperidine-2,6-dione (98.1 mg, 189 μmol, 1.00 eq, TFA). The mixture was stirred at 25 °C for 2 h. LC-MS showed the desired mass was detected. The reaction mixture was quenched by addition H₂O 3 mL, and then extracted with EtOAc (3 mL*3). The combined organic layers were dried over Na₂SO₄, filtered and concentrated under reduced pressure. The residue was purified by prep-HPLC (column: Phenomenex luna C18 100*40 mm*3 um; mobile phase: [H₂O (0.2% FA)-ACN]; gradient: 15%-50% B over 8.0 min) to give the title compound (Compound 465) (20.1 mg, 25.3 μmol, 13.3% yield, 100% purity) as a white solid. LCMS (ES⁺): m/z 794.4 [M+H]⁺.

### Example 598. Synthesis of trans-4-((5-fluoro-4-(3-(2-oxopyridin-1 (2H)-yl)phenyl)pyrimidin-2-yl)amino)cyclohexyl 4-((4-(3-(2,6-dioxopiperidin-3-yl)-1-methyl-1H-indazol-6-yl)piperidin-1-yl)methyl)piperidine-1-carboxylate (Compound 467)

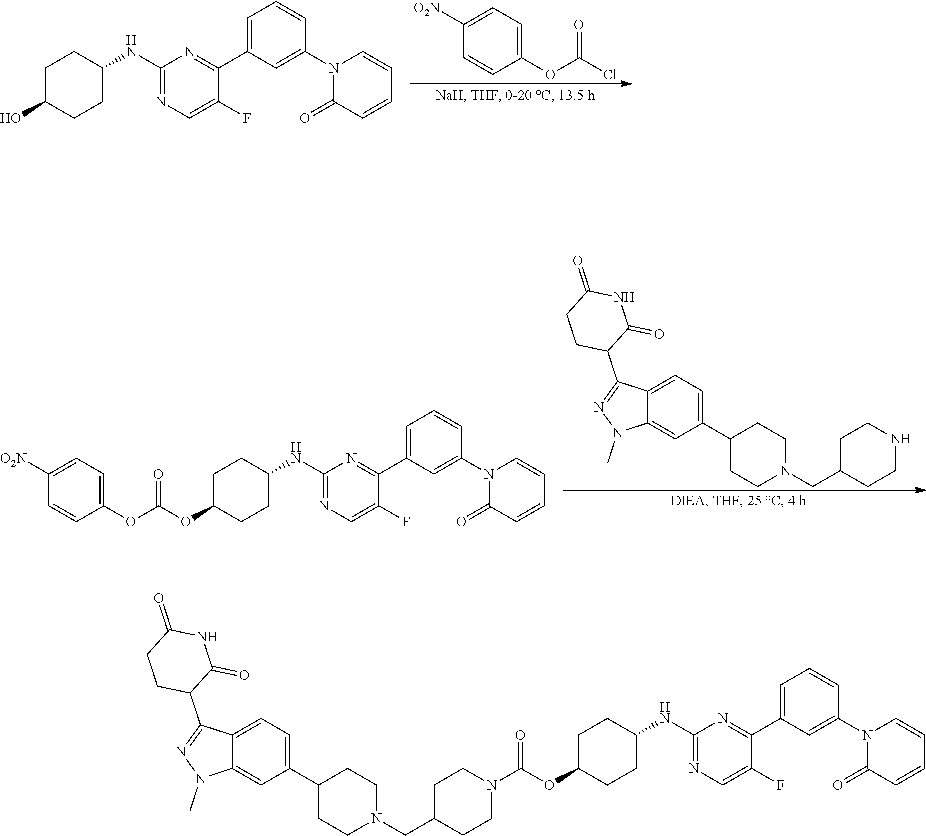 [SMILES: O=c1ccccn1-c1cccc(-c2nc(N[C@H]3CC[C@H](O)CC3)ncc2F)c1 ; O=C(Cl)Oc1ccc([N+](=O)[O-])cc1 ; O=C(Oc1ccc([N+](=O)[O-])cc1)O[C@H]1CC[C@H](Nc2ncc(F)c(-c3cccc(-n4ccccc4=O)c3)n2)CC1 ; Cn1nc(C2CCC(=O)NC2=O)c2ccc(C3CCN(CC4CCNCC4)CC3)cc21 ; Cn1nc(C2CCC(=O)NC2=O)c2ccc(C3CCN(CC4CCN(C(=O)O[C@H]5CC[C@H](Nc6ncc(F)c(-c7cccc(-n8ccccc8=O)c7)n6)CC5)CC4)CC3)cc21]

Synthesis of trans-4-((5-fluoro-4-(3-(2-oxopyridin-1 (2H)-yl)phenyl)pyrimidin-2-yl)amino)cyclohexyl (4-nitrophenyl) carbonate. To a solution of trans-1-(3-(5-fluoro-2-((4-hydroxycyclohexyl)amino)pyrimidin-4-yl)phenyl)pyridin-2 (1H)-one (5 g, 13.14 mmol, 1 eq) in THF (50 mL) was added NaH (1.58 g, 39.4 mmol, 60.0% purity, 3.00 eq) at 0 °C under N₂ atmosphere. The mixture was stirred at 0 °C for 1.5 h under N₂ atmosphere. Then a solution of (4-nitrophenyl) carbonochloridate (10.6 g, 52.6 mmol, 4.00 eq) in THF (50 mL) was added to the mixture at 0 °C. The mixture was slowly warmed to 20 °C and stirred at 20 °C for 12 h. LCMS showed trans-1-(3-(5-fluoro-2-((4-hydroxycyclohexyl)amino)pyrimidin-4-yl)phenyl)pyridin-2 (1H)-one (5 g, 13.14 mmol, 1 eq) was consumed completely and the desired target MS was detected. The reaction was concentrated under reduced pressure to give a residue. The residue was poured into saturated NH₄Cl solution (100 mL) and extracted with EtOAc (100 mL*3). The combined organic layers were dried over anhydrous Na₂SO₄, filtered and the filtrate was concentrated under reduced pressure to give a residue. The residue was purified by MPLC (SiO₂, Petroleum ether:Ethyl acetate=1:0 to 0:1) to give the title compound (430 mg, 788 μmol, 6.00% yield) as a yellow oil. LCMS (ES⁺): m/z 546.2 [M+H]⁺.

Synthesis of trans-4-((5-fluoro-4-(3-(2-oxopyridin-1 (2H)-yl)phenyl)pyrimidin-2-yl)amino)cyclohexyl 4-((4-(3-(2,6-dioxopiperidin-3-yl)-1-methyl-1H-indazol-6-yl)piperidin-1-yl)methyl)piperidine-1-carboxylate (Compound 467). To a solution of 3-(1-methyl-6-(1-(piperidin-4-ylmethyl)piperidin-4-yl)-1H-indazol-3-yl)piperidine-2,6-dione (200 mg, 372 mol, 1.00 eq, TFA) in THF (5 mL) was added DIEA (241 mg, 1.86 mmol, 325 μL, 5.00 eq) at 25 °C. Then trans-4-((5-fluoro-4-(3-(2-oxopyridin-1 (2H)-yl)phenyl)pyrimidin-2-yl)amino)cyclohexyl (4-nitrophenyl) carbonate (194923) (122 mg, 223 μmol, 0.600 eq) was added to the reaction mixture at 25 °C. The mixture was stirred at 25 °C for 4 h. LCMS showed 3-(1-methyl-6-(1-(piperidin-4-ylmethyl)piperidin-4-yl)-1H-indazol-3-yl)piperidine-2,6-dione was consumed completely and the desired mass was detected. The mixture was filtered and the filtrate was concentrated under reduced pressure to give a residue. The residue was purified by prep-HPLC (column: Phenomenex luna C18 100*40 mm*5 um; mobile phase: [1H₂O (0.2% FA)-ACN]; gradient: 15%-50% B over 8.0 min) to give the title compound (Compound 467) (48.2 mg, 50.6 μmol, 13.6% yield, 92.0% purity, FA) as a white solid. LCMS (ES⁺): m/z 830.5 [M+H]⁺.

### Example 599. Synthesis of trans-4-((5-fluoro-4-(3-(2-oxopyridin-1 (2H)-yl)phenyl)pyrimidin-2-yl)amino)cyclohexyl 4-((4-(3-((R)-2,6-dioxopiperidin-3-yl)-1-methyl-1H-indazol-6-yl)piperidin-1-yl)methyl)piperidine-1-carboxylate (Compound 639) and trans-4-((5-fluoro-4-(3-(2-oxopyridin-1 (2H)-yl)phenyl)pyrimidin-2-yl)amino)cyclohexyl 4-((4-(3-((S)-2,6-dioxopiperidin-3-yl)-1-methyl-1H-indazol-6-yl)piperidin-1-yl)methyl)piperidine-1-carboxylate (Compound 638) (The Two Isomers were Assigned Arbitrarily)

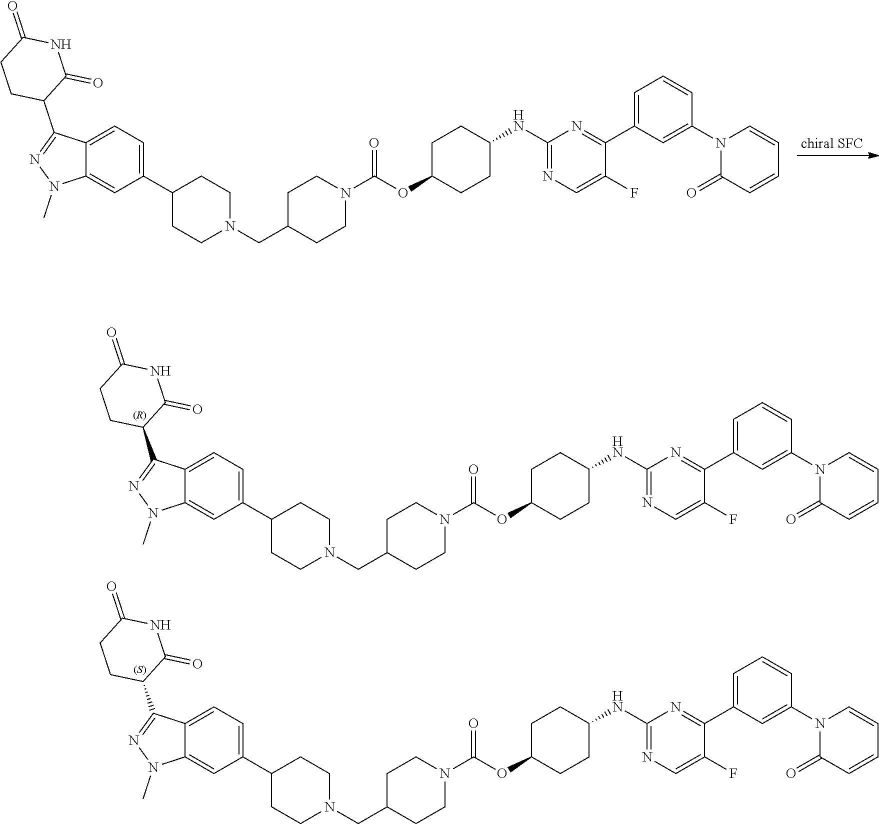

Synthesis of trans-4-((5-fluoro-4-(3-(2-oxopyridin-1 (2H)-yl)phenyl)pyrimidin-2-yl)amino)cyclohexyl 4-((4-(3-((R)-2,6-dioxopiperidin-3-yl)-1-methyl-1H-indazol-6-yl)piperidin-1-yl)methyl)piperidine-1-carboxylate (Compound 639) and trans-4-((5-fluoro-4-(3-(2-oxopyridin-1 (2H)-yl)phenyl)pyrimidin-2-yl)amino)cyclohexyl 4-((4-(3-((S)-2,6-dioxopiperidin-3-yl)-1-methyl-1H-indazol-6-yl)piperidin-1-yl)methyl)piperidine-1-carboxylate (Compound 638). Trans-4-((5-fluoro-4-(3-(2-oxopyridin-1 (2H)-yl)phenyl)pyrimidin-2-yl)amino)cyclohexyl 4-((4-(3-(2,6-dioxopiperidin-3-yl)-1-methyl-1H-indazol-6-yl)piperidin-1-yl)methyl)piperidine-1-carboxylate (Compound 467) (350 mg, 422 mol, 1.00 eq) was further separated by SFC (column: REGIS(S,S)WHELK-O1 (250 mm*25 mm, 10 um); mobile phase: [CO₂-IPA:ACN=1:1 (0.1% IPAm)]; B %: 60%, isocratic elution mode) to give the title compound (Compound 639) (94.0 mg, 113 μmol, 26.9% yield, 100% purity, 96.8% ee) (retention time: 1.42, peak 1) as a white solid. LCMS (ES⁺): m/z 830.5 [M+H]⁺/415.8 and give the title compound (Compound 638) (57.3 mg, 69.0 μmol, 16.4% yield, 99.9% purity, 96.4% ee) (retention time: 2.53, peak 2) as a white solid. LCMS (ES⁺): m/z 830.5 [M+H]⁺.

### Example 600. Synthesis of trans-4-((5-fluoro-4-(3-(2-oxopyridin-1 (2H)-yl)phenyl)pyrimidin-2-yl)amino)cyclohexyl 4-(4-(4-((2,6-dioxopiperidin-3-yl)amino)-2-fluorophenyl)piperazin-1-yl)piperidine-1-carboxylate (Compound 475)

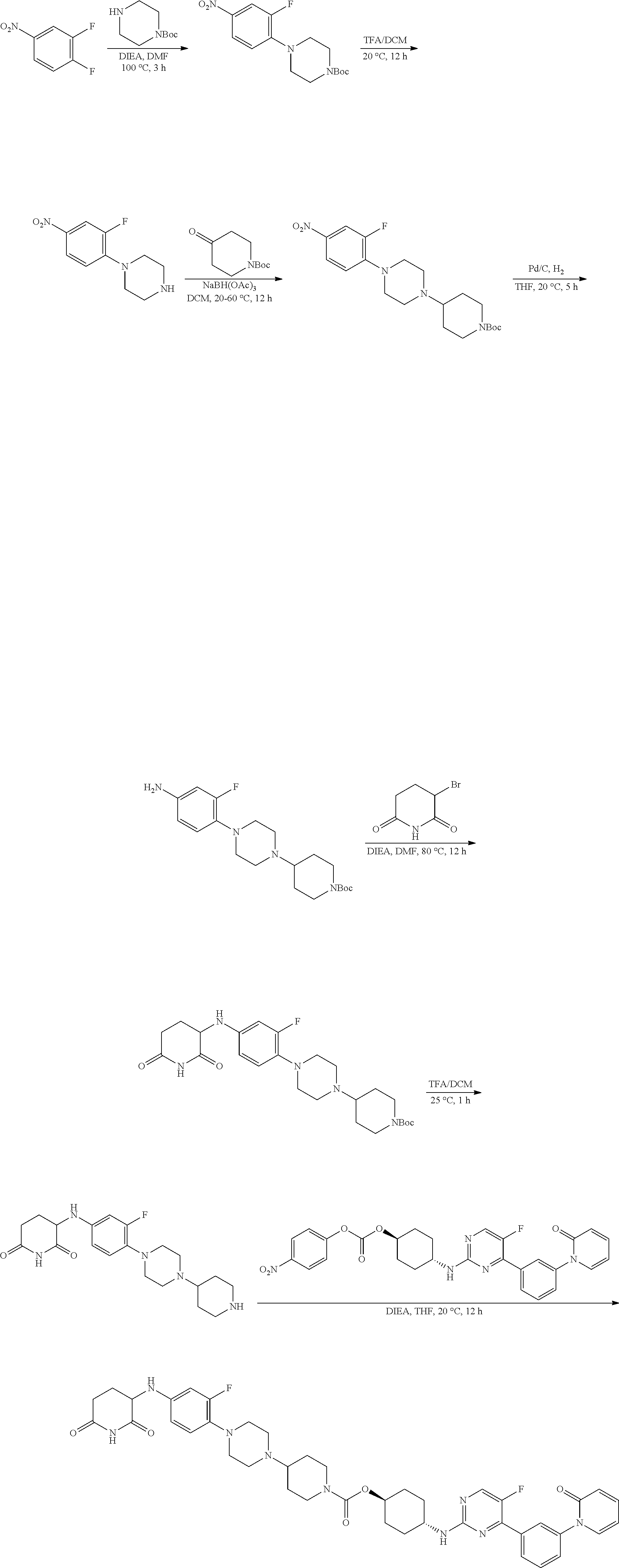

Synthesis of tert-butyl 4-(2-fluoro-4-nitrophenyl)piperazine-1-carboxylate. To a solution of 1,2-difluoro-4-nitrobenzene (20.0 g, 126 mmol, 13.9 mL, 1.00 eq) and tert-butyl piperazine-1-carboxylate (23.4 g, 126 mmol, 1.00 eq) in DMF (50 mL) was added DIEA (48.7 g, 377 mmol, 65.7 mL, 3.00 eq) at 20 °C. The mixture was stirred at 100 °C for 3 h. LC-MS showed that 1,2-difluoro-4-nitrobenzene was consumed completely and desired mass was detected. The mixture was filtered and filtrate cake was concentrated under reduced pressure to remove solvent to give the title compound (26.0 g, crude) as a yellow solid. LCMS (ES⁺): m/z 326.1 [M+H]⁺

Synthesis of 1-(2-fluoro-4-nitrophenyl)piperazine. To a solution of tert-butyl 4-(2-fluoro-4-nitrophenyl)piperazine-1-carboxylate (26.0 g, 79.9 mmol, 1.00 eq) in DCM (60 mL) was added TFA (30.7 g, 269 mmol, 20.0 mL, 3.37 eq) at 20 °C. The mixture was stirred at 20 °C for 12 h. LC-MS showed that tert-butyl 4-(2-fluoro-4-nitrophenyl)piperazine-1-carboxylate was consumed completely and desired mass was detected. The reaction mixture was concentrated under reduced pressure to remove solvent to give the title compound (26.0 g, crude, TFA) as a brown oil. LCMS (ES⁺): m/z 226.0 [M+H]⁺

Synthesis of tert-butyl 4-(4-(2-fluoro-4-nitrophenyl)piperazin-1-yl)piperidine-1-carboxylate. To a solution of 1-(2-fluoro-4-nitrophenyl)piperazine (26.0 g, 76.6 mmol, 1.00 eq, TFA) and tert-butyl 4-oxopiperidine-1-carboxylate (45.8 g, 230 mmol, 3.00 eq) in DCM (500 mL) was added NaBH(OAc)₃ (40.6 g, 192 mmol, 2.50 eq) at 20 °C. The mixture was stirred at 60 °C for 12 h. LC-MS showed that 1-(2-fluoro-4-nitrophenyl)piperazine was consumed completely and desired mass was detected. The residue was diluted with H₂O (500 mL) and extracted with EtOAc (300 mL*5). The combined organic layers were washed with brine 500 mL, dried over anhydrous Na₂SO₄, filtered and concentrated under reduced pressure to give a residue. The residue was purified by column chromatography (SiO₂, Petroleum ether/Ethyl acetate=1/0 to 1/4) to give the title compound (31.0 g, 75.7 mmol, 98.7% yield, 99.7% purity) as an orange solid. LCMS (ES⁺): m/z 409.2 [M+H]⁺

Synthesis of tert-butyl 4-(4-(4-amino-2-fluorophenyl)piperazin-1-yl)piperidine-1-carboxylate To a mixture of 10% Pd/C (3.13 g, 14.7 mmol, 50.0% purity, 1.20 eq) in THF (50 mL) was added tert-butyl 4-(4-(2-fluoro-4-nitrophenyl)piperazin-1-yl)piperidine-1-carboxylate (5.00 g, 12.2 mmol, 1.00 eq) at 20 °C. The mixture was stirred at 20 °C for 5 h under H₂ (15 Psi). LC-MS showed tert-butyl 4-(4-(2-fluoro-4-nitrophenyl)piperazin-1-yl)piperidine-1-carboxylate was consumed completely and desired mass was detected. The mixture was filtered and filtrate was concentrated under reduced pressure to remove solvent to give the title compound (4.60 g, crude) as a pink oil. LCMS (ES⁺): m/z 379.2 [M+H]⁺

Synthesis of tert-butyl-4-(4-(4-((2,6-dioxopiperidin-3-yl)amino)-2-fluorophenyl)piperazin-1-yl)piperidine-1-carboxylate To a solution of tert-butyl 4-(4-(4-amino-2-fluorophenyl)piperazin-1-yl)piperidine-1-carboxylate (4.60 g, 12.2 mmol, 1.00 eq) and 3-bromopiperidine-2,6-dione (9.33 g, 48.6 mmol, 4.00 eq) in DMF (40 mL was added DIEA (4.71 g, 36.5 mmol, 6.35 mL, 3.00 eq) at 20 °C. The mixture was stirred at 80 °C for 12 h. LC-MS showed tert-butyl 4-(4-(4-amino-2-fluorophenyl)piperazin-1-yl)piperidine-1-carboxylate was remained and desired mass was detected. The residue was diluted with H₂O (80 mL) and extracted with EtOAc (60 mL*8). The combined organic layers were washed with brine (80 mL), dried over anhydrous Na₂SO₄, filtered and concentrated under reduced pressure to give a residue. The residue was purified by column chromatography (SiO2, Petroleum ether/THF=1/0 to 0/1) to give the title compound (3.0 g, crude) as a green solid. LCMS (ES⁺): m/z 490.2 [M+H]⁺

Synthesis of 3-((3-fluoro-4-(4-(piperidin-4-yl)piperazin-1-yl)phenyl)amino)piperidine-2,6-dione To a solution of tert-butyl-4-(4-(4-((2,6-dioxopiperidin-3-yl)amino)-2-fluorophenyl)piperazin-1-yl)piperidine-1-carboxylate (200 mg, 409 μmol, 1.00 eq) in DCM (3 mL) was added TFA (1.54 g, 13.5 mmol, 1.00 mL, 33.0 eq) at 20 °C. The mixture was stirred at 20 °C for 1 h. LC-MS showed that tert-butyl-4-(4-(4-((2,6-dioxopiperidin-3-yl)amino)-2-fluorophenyl)piperazin-1-yl)piperidine-1-carboxylate was consumed completely and desired mass was detected. The reaction mixture was concentrated under reduced pressure to remove solvent to give the title compound (200 mg, crude, TFA) as a green solid. LCMS (ES⁺): m/z 390.2 [M+H]⁺

Synthesis of trans-4-((5-fluoro-4-(3-(2-oxopyridin-1 (2H)-yl)phenyl)pyrimidin-2-yl)amino)cyclohexyl 4-(4-(4-((2,6-dioxopiperidin-3-yl)amino)-2-fluorophenyl)piperazin-1-yl)piperidine-1-carboxylate (Compound 475). To a solution of rac-3-((3-fluoro-4-(4-(piperidin-4-yl)piperazin-1-yl)phenyl)amino)piperidine-2,6-dione (200 mg, 397 μmol, 1.00 eq, TFA) and trans-(1r,4r)-4-((5-fluoro-4-(3-(2-oxopyridin-1 (2H)-yl)phenyl)pyrimidin-2-yl)amino)cyclohexyl (4-nitrophenyl) carbonate (217 mg, 397 μmol, 1.00 eq) in THF (2 mL) was added DIEA (51.3 mg, 397 μmol, 69.2 μL, 1.00 eq) at 20 °C. The mixture was stirred at 20 °C for 12 h. LC-MS showed that 3-((3-fluoro-4-(4-(piperidin-4-yl)piperazin-1-yl)phenyl)amino)piperidine-2,6-dione was consumed completely and desired mass was detected. The reaction mixture was concentrated under reduced pressure to remove solvent. The residue was purified by prep-HPLC (column: Phenomenex Luna C18 100*30 mm*3 um; mobile phase: [H₂O (0.2% FA)-ACN]; gradient: 5%-35% B over 8.0 min) to give the title compound (Compound 475) (105 mg, 131 μmol, 32.9% yield, 98.9% purity) as a white solid. LCMS (ES⁺): m/z 796.3 [M+H]⁺

### Example 601. Synthesis of trans-4-((5-fluoro-4-(3-(2-oxopyridin-1 (2H)-yl)phenyl)pyrimidin-2-yl)amino)cyclohexyl 4-(4-((2,6-dioxopiperidin-3-yl)amino)-2-fluorophenyl)-[1,4′-bipiperidine]-1′-carboxylate (Compound 474)

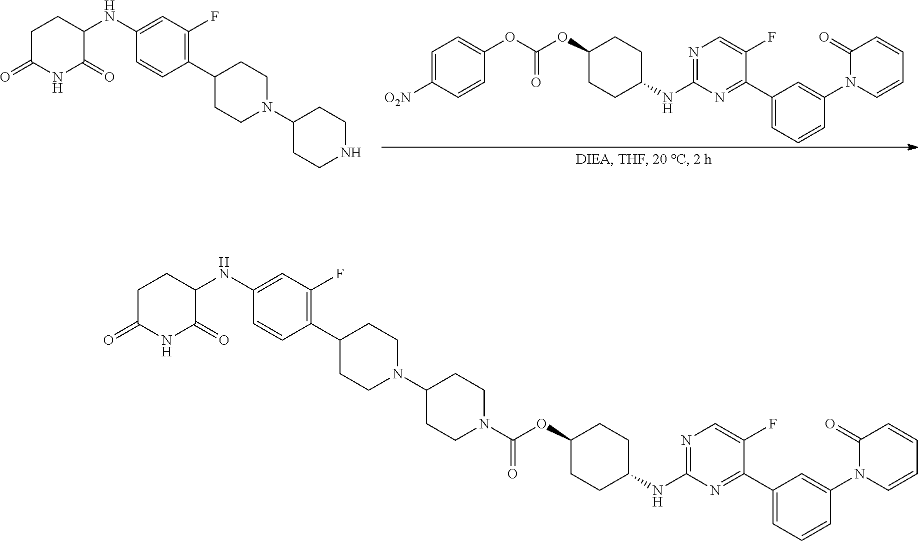 [SMILES: O=C1CCC(Nc2ccc(C3CCN(C4CCNCC4)CC3)c(F)c2)C(=O)N1 ; O=C(Oc1ccc([N+](=O)[O-])cc1)O[C@H]1CC[C@H](Nc2ncc(F)c(-c3cccc(-n4ccccc4=O)c3)n2)CC1 ; O=C1CCC(Nc2ccc(C3CCN(C4CCN(C(=O)O[C@H]5CC[C@H](Nc6ncc(F)c(-c7cccc(-n8ccccc8=O)c7)n6)CC5)CC4)CC3)c(F)c2)C(=O)N1]

Synthesis of trans-4-((5-fluoro-4-(3-(2-oxopyridin-1 (2H)-yl)phenyl)pyrimidin-2-yl)amino)cyclohexyl 4-(4-((2,6-dioxopiperidin-3-yl)amino)-2-fluorophenyl)-[1,4′-bipiperidine]-1′-carboxylate (Compound 474). To a solution of 3-((4-([1,4′-bipiperidin]-4-yl)-3-fluorophenyl)amino)piperidine-2,6-dione (200 mg, 398 μmol, 1.00 eq, TFA) in THF (4 mL) was added trans-(1r,4r)-4-((5-fluoro-4-(3-(2-oxopyridin-1 (2H)-yl)phenyl)pyrimidin-2-yl)amino)cyclohexyl (4-nitrophenyl) carbonate (209 mg, 383 μmol, 9.61e-1 eq) and DIEA (103 mg, 796 μmol, 139 μL, 2.00 eq) at 20 °C. The mixture was stirred at 20 °C for 2 h. LC-MS showed that 3-((4-([1,4′-bipiperidin]-4-yl)-3-fluorophenyl)amino)piperidine-2,6-dione was not consumed completely and one main peak with the desired mass was detected. The mixture was concentrated under reduced pressure. The residue was purified by prep-HPLC (column: Phenomenex luna C18 100*40 mm*5 um; mobile phase: [H₂O (0.2% FA)-ACN]; gradient: 10%-50% B over 8.0 min) to give the title compound (Compound 474) (125 mg, 153 μmol, 38.3% yield, 98.4% purity, 0.2 FA) as a pale yellow solid. LCMS (ES⁺): m/z 795.3 [M+H]⁺.

### Example 602. Synthesis of trans-4-((4-([1,1′-biphenyl]-3-yl)-5-fluoropyrimidin-2-yl)amino)cyclohexyl 4-(2-(4-(4-((2,6-dioxopiperidin-3-yl)amino)-2-fluorophenyl)piperidin-1-yl)ethyl)piperidine-1-carboxylate (Compound 469)

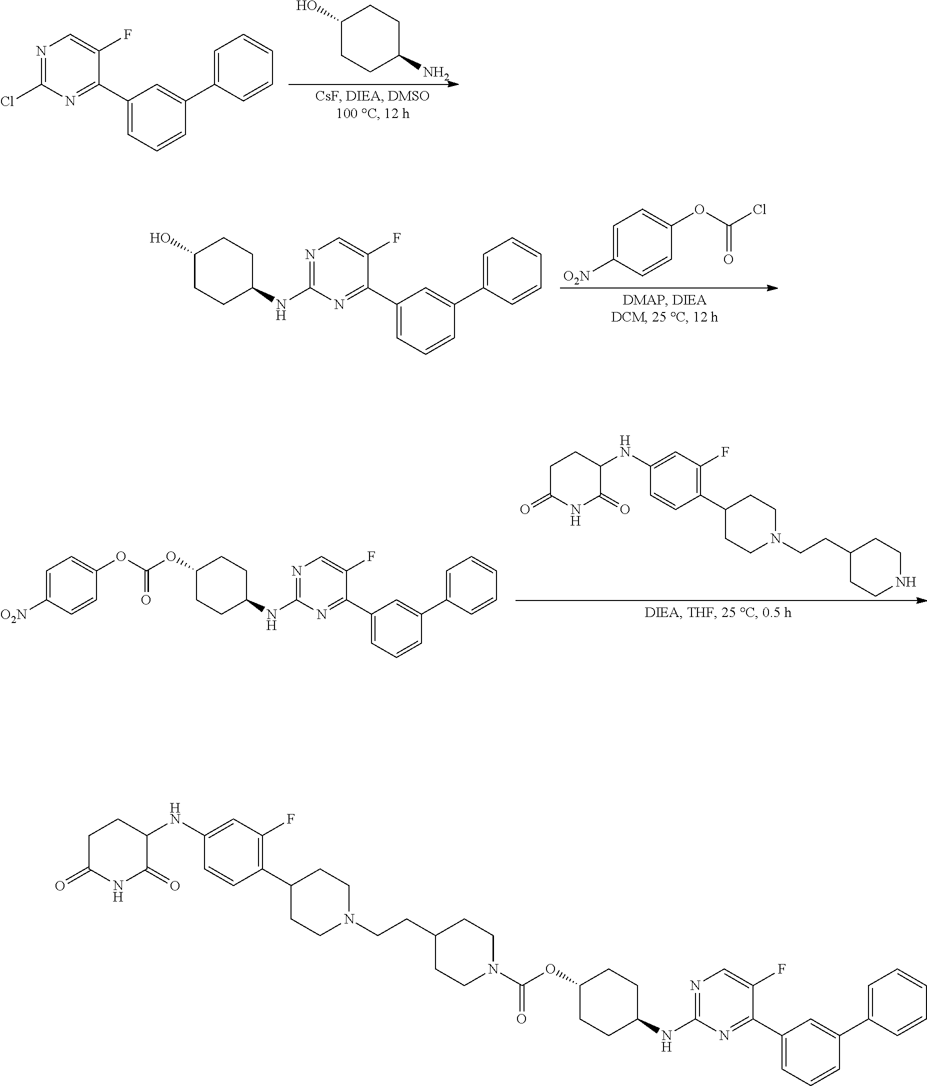

Synthesis of trans-4-((4-([1,1′-biphenyl]-3-yl)-5-fluoropyrimidin-2-yl)amino)cyclohexan-1-ol. To a solution of 4-([1,1′-biphenyl]-3-yl)-2-chloro-5-fluoropyrimidine (1.00 g, 3.51 mmol, 1.00 eq) and trans-4-aminocyclohexan-1-ol (607 mg, 5.27 mmol, 1.50 eq) in DMSO (15 mL) were added CsF (800 mg, 5.27 mmol, 1.50 eq) and DIEA (1.36 g, 10.5 mmol, 1.84 mL, 3.00 eq) at 25 °C. The mixture was stirred at 100 °C for 12 h. LCMS showed 4-([1,1′-biphenyl]-3-yl)-2-chloro-5-fluoropyrimidine was consumed and the desired target MS was detected. A solid was precipitated from the reaction mixture, and the reaction mixture was filtered and the filter cake was dried under reduced pressure to give the title compound (1.20 g, crude) as a white solid. LCMS (ES⁺): m/z 364.2 [M+H]⁺.

Synthesis of trans-4-((4-([1,1′-biphenyl]-3-yl)-5-fluoropyrimidin-2-yl)amino)cyclohexyl (4-nitrophenyl) carbonate. To a solution of trans-4-((4-([1,1′-biphenyl]-3-yl)-5-fluoropyrimidin-2-yl)amino)cyclohexan-1-ol (1.00 g, 2.75 mmol, 1.00 eq) and 4-nitrophenyl carbonochloridate (3.33 g, 16.5 mmol, 6.00 eq) in DCM (15 mL) were added DIEA (711 mg, 5.50 mmol, 959 μL, 2.00 eq) and DMAP (336 mg, 2.75 mmol, 1.00 eq) at 25 °C. The resulting mixture was stirred at 25 °C for 12 h. LCMS showed trans-4-((4-([1,1′-biphenyl]-3-yl)-5-fluoropyrimidin-2-yl)amino)cyclohexan-1-ol was consumed and the desired mass was detected. H₂O (10 mL) was added to the solution, then the mixture was extracted with DCM (10 mL*3), the combined organic layers were dried over Na₂SO₄, filtered and the filtrate was concentrated under reduced pressure to give a residue. The residue was purified by prep-TLC (SiO2, PE/THF=4/1) to give the title compound (550 mg, 1.04 mmol, 37.82% yield) as a yellow solid. LCMS (ES⁺): m/z 529.2 [M+H]⁺.

Synthesis of trans-4-((4-([1,1′-biphenyl]-3-yl)-5-fluoropyrimidin-2-yl)amino)cyclohexyl 4-(2-(4-(4-((2,6-dioxopiperidin-3-yl)amino)-2-fluorophenyl)piperidin-1-yl)ethyl)piperidine-1-carboxylate (Compound 469). To a solution of 3-((3-fluoro-4-(1-(2-(piperidin-4-yl)ethyl)piperidin-4-yl)phenyl)amino)piperidine-2,6-dione (103 mg, 194 μmol, 1.71 eq, TFA) in THF (3 mL) was added DIEA (44.0 mg, 341 μmol, 59.3 μL, 3.00 eq) at 25 °C, and then trans-4-((4-([1,1′-biphenyl]-3-yl)-5-fluoropyrimidin-2-yl)amino)cyclohexyl (4-nitrophenyl) carbonate (100 mg, 114 μmol, 1.00 eq) in THF (3 mL) was added to above mixture at 25 °C. The resulting mixture was stirred at 25 °C for 0.5 h. LCMS showed 3-((3-fluoro-4-(1-(2-(piperidin-4-yl)ethyl)piperidin-4-yl)phenyl)amino)piperidine-2,6-dione was consumed and the desired mass was detected. The mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 40%-75% B over 8.0 min) to give the title compound (Compound 469) (17.5 mg, 62.0 μmol, 19.13% yield) as a yellow solid. LCMS (ES⁺): m/z 805.8 [M+H]⁺.

### Example 603. Synthesis of trans-3-((3-fluoro-4-(1-((1-(4-((5-fluoro-4-(3-(4-methyl-2-oxopyridin-1 (2H)-yl)phenyl)pyrimidin-2-yl)amino)cyclohexane-1-carbonyl)piperidin-4-yl)methyl)piperidin-4-yl)phenyl)amino)piperidine-2,6-dione (Compound 473)

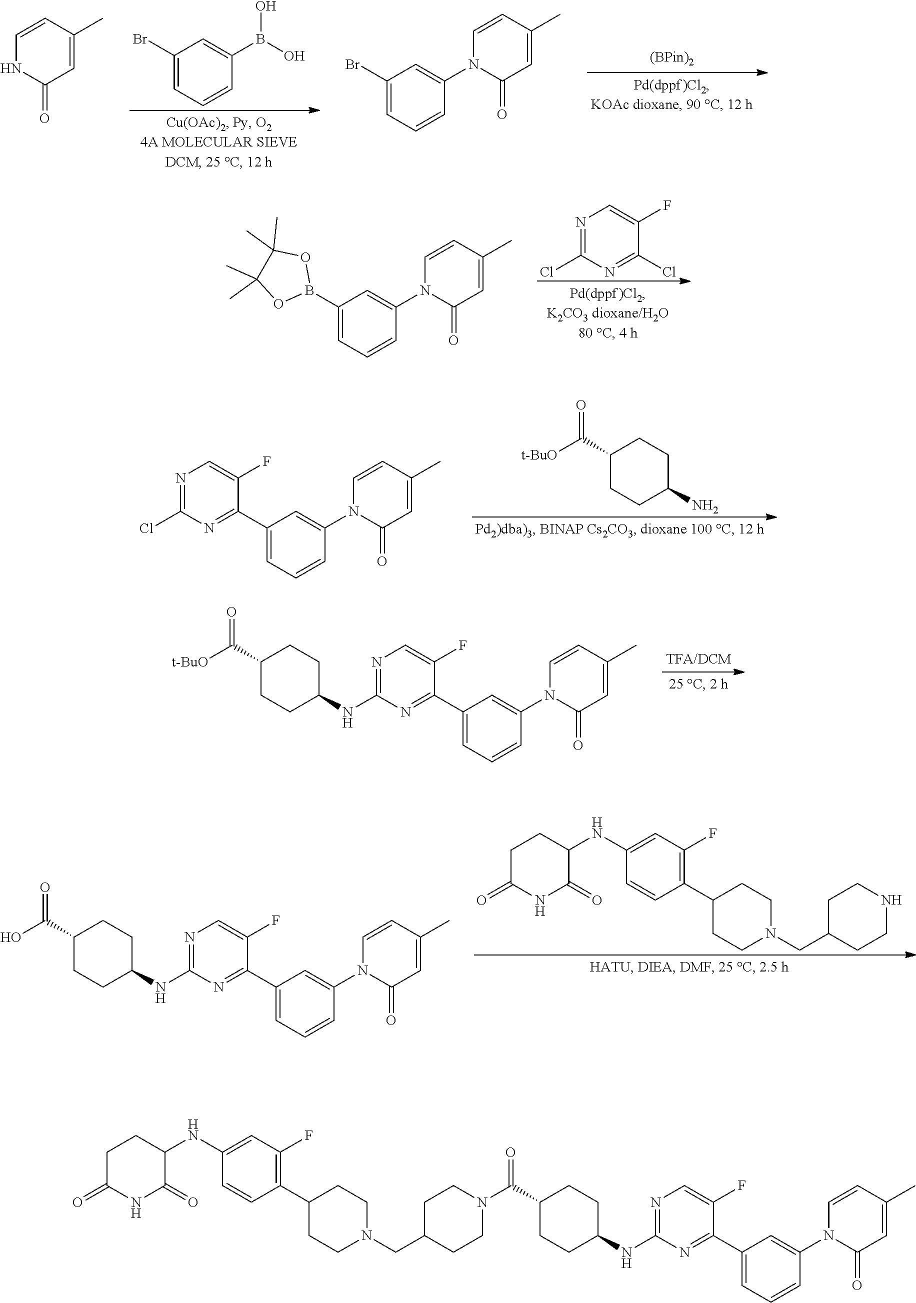

Synthesis of 1-(3-bromophenyl)-4-methylpyridin-2 (1H)-one. To a solution of (3-bromophenyl)boronic acid (55.2 g, 275 mmol, 2.00 eq) and 4-methylpyridin-2 (1H)-one (15.0 g, 137 mmol, 1.00 eq) in DCM (1000 mL) were added Py (21.8 g, 275 mmol, 22.2 mL, 2.00 eq), 4 A MOLECULAR SIEVE (3.00 g) and Cu(OAc)₂ (2.50 g, 13.8 mmol, 0.100 eq) at 25 °C. The mixture was stirred at 25 °C for 12 h under O₂ (15 psi). LCMS showed the desired mass was detected. To the mixture was added H₂O (1000 mL) and the mixture was extracted with DCM (500 mL*3). The combined organic layers were washed with brine (100 mL*3), dried over Na₂SO₄, filtered and the filtrate was concentrated under reduced pressure to give a residue. The residue was purified by MPLC (SiO₂, PE/EtOAc=1/0 to 1/1) to give the title compound (30.0 g, 114 mmol, 82.6% yield) as a pink solid. LCMS (ES+): m/z 263.9 [M+H]⁺

Synthesis of 4-methyl-1-(3-(4,4,5,5-tetramethyl-1,3,2-dioxaborolan-2-yl)phenyl)pyridin-2 (1H)-one. Three batches in parallel were set up. To a solution of 1-(3-bromophenyl)-4-methylpyridin-2 (1H)-one (6.00 g, 22.7 mmol, 1.00 eq) and 4,4,4′,4′,5,5,5′,5′-octamethyl-2,2′-bi(1,3,2-dioxaborolane) (6.93 g, 27.3 mmol, 1.20 eq) in dioxane (50 mL) were added Pd(dppf)Cl₂ (1.66 mg, 2.27 mmol, 0.100 eq) and KOAc (4.47 g, 45.3 mmol, 2.00 eq) at 25 °C under N₂, the mixture was stirred at 90 °C for 12 h under N₂. LCMS showed 1-(3-bromophenyl)-4-methylpyridin-2 (1H)-one was consumed and the desired mass was detected. The mixture was concentrated under reduced pressure to give the title compound (7.00 g, crude) as a black oil. LCMS (ES⁺): m/z 312.0 [M+H]⁺.

Synthesis of 1-(3-(2-chloro-5-fluoropyrimidin-4-yl)phenyl)-4-methylpyridin-2 (1H)-one. To a solution of 4-methyl-1-(3-(4,4,5,5-tetramethyl-1,3,2-dioxaborolan-2-yl)phenyl)pyridin-2 (1H)-one (6.30 g, 20.3 mmol, 1.00 eq) and 2,4-dichloro-5-fluoropyrimidine (3.72 g, 22.3 mmol, 1.10 eq) in dioxane (150 mL) and H₂O (15 mL) were added K₂CO₃ (8.39 g, 60.7 mmol, 3.00 eq) and Pd(dppf)Cl₂ (1.48 g, 2.02 mmol, 0.100 eq) at 25 °C under N₂. The mixture was stirred at 80 °C for 2 h under N₂. LCMS showed 4-methyl-1-(3-(4,4,5,5-tetramethyl-1,3,2-dioxaborolan-2-yl)phenyl)pyridin-2 (1H)-one remained and the desired mass was detected. Then 2,4-dichloro-5-fluoropyrimidine (1.35 g, 0.400 eq) and Pd(dppf)Cl₂ (740 mg, 0.0500 eq) were added to the mixture at 25 °C under N₂, the mixture was stirred at 80 °C for another 2 h under N₂. LCMS showed 4-methyl-1-(3-(4,4,5,5-tetramethyl-1,3,2-dioxaborolan-2-yl)phenyl)pyridin-2 (1H)-one was consumed and the desired mass was detected. To the mixture was added H₂O (150 mL) and the mixture was extracted with EtOAc (100 mL*3). The combined organic layers were washed with brine (100 mL*3), dried over Na₂SO₄, filtered and the filtrate was concentrated under reduced pressure to give a residue. The residue was purified by MPLC (SiO₂, PE/EtOAc=1/0 to 1/1) to give the title compound (5.50 g, 17.4 mmol, 86.0% yield) as a pink solid. LCMS (ES+): m/z 316.0 [M+H]⁺.

Synthesis of trans-tert-butyl 4-((5-fluoro-4-(3-(4-methyl-2-oxopyridin-1 (2H)-yl)phenyl)pyrimidin-2-yl)amino)cyclohexane-1-carboxylate. To a solution of 1-(3-(2-chloro-5-fluoropyrimidin-4-yl)phenyl)-4-methylpyridin-2 (1H)-one (3.00 g, 9.50 mmol, 1.00 eq) and trans-tert-butyl 4-aminocyclohexane-1-carboxylate (2.84 g, 14.3 mmol, 1.50 eq) in dioxane (100 mL) were added BINAP (532 mg, 855 μmol, 0.0900 eq), Cs₂CO₃ (6.19 g, 19.0 mmol, 2.00 eq) and Pd₂(dba)₃ (522 mg, 570 μmol, 0.0600 eq) at 25 °C under N₂ atmosphere. The mixture was stirred at 100 °C for 12 h under N₂ atmosphere. LCMS showed 1-(3-(2-chloro-5-fluoropyrimidin-4-yl)phenyl)-4-methylpyridin-2 (1H)-one was consumed and the desired mass was detected. To the mixture was added H₂O (80 mL) and the mixture was extracted with EtOAc (80 mL*3). The combined organic layers were washed with brine (50 mL*3), dried over Na₂SO₄, filtered and the filtrate was concentrated under reduced pressure to give a residue. The residue was purified by MPLC (SiO₂, PE/EtOAc=1/0 to 1/1) to give the title compound (4.00 g, 8.36 mmol, 88.0% yield) as a white solid. LCMS (ES+): m/z 479.3 [M+H]⁺

Synthesis of trans-4-((5-fluoro-4-(3-(4-methyl-2-oxopyridin-1 (2H)-yl)phenyl)pyrimidin-2-yl)amino)cyclohexane-1-carboxylic acid. A solution of trans-tert-butyl 4-((5-fluoro-4-(3-(4-methyl-2-oxopyridin-1 (2H)-yl)phenyl)pyrimidin-2-yl)amino)cyclohexane-1-carboxylate (200 mg, 418 μmol, 1.00 eq) in DCM (3 mL) and TFA (1 mL) was stirred at 25 °C for 2 h. LCMS showed trans-tert-butyl 4-((5-fluoro-4-(3-(4-methyl-2-oxopyridin-1 (2H)-yl)phenyl)pyrimidin-2-yl)amino)cyclohexane-1-carboxylate was consumed and the desired mass was detected. The mixture was concentrated under reduced pressure to give the title compound (177 mg, crude) as a yellow oil. LCMS (ES+): m/z 423.2 [M+H]⁺

Synthesis of trans-3-((3-fluoro-4-(1-((1-(4-((5-fluoro-4-(3-(4-methyl-2-oxopyridin-1 (2H)-yl)phenyl)pyrimidin-2-yl)amino)cyclohexane-1-carbonyl)piperidin-4-yl)methyl)piperidin-4-yl)phenyl)amino)piperidine-2,6-dione (Compound 473). To a solution of trans-4-((5-fluoro-4-(3-(4-methyl-2-oxopyridin-1 (2H)-yl)phenyl)pyrimidin-2-yl)amino)cyclohexane-1-carboxylic acid (177 mg, 419 μmol, 0.800 eq) in DMF (8 mL) were added DIEA (338 mg, 2.63 mmol, 455 μL, 5.00 eq) and HATU (199 mg, 524 μmol, 1.00 eq) at 25 °C. Then to the mixture was added 3-((3-fluoro-4-(1-(piperidin-4-ylmethyl)piperidin-4-yl)phenyl)amino)piperidine-2,6-dione (271 mg, 524 μmol, 1.00 eq, TFA) at 25 °C. The mixture was stirred at 25 °C for 2.5 h. LCMS showed trans-4-((5-fluoro-4-(3-(4-methyl-2-oxopyridin-1 (2H)-yl)phenyl)pyrimidin-2-yl)amino)cyclohexane-1-carboxylic acid was consumed completely and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to give the title compound (Compound 473) (22.3 mg, 27.2 μmol, 5.19% yield, 98.3% purity) as a white solid. LCMS (ES⁺): m/z 807.4 [M+H]⁺

### Example 604. Synthesis of trans-3-[3-fluoro-4-[1-[[1-[4-[[5-fluoro-4-[3-(4-methyl-2-oxo-1-pyridyl)phenyl]pyrimidin-2-yl]amino]cyclohexanecarbonyl]-4-hydroxy-4-piperidyl]methyl]-4-piperidyl]anilino]piperidine-2,6-dione (Compound 472)

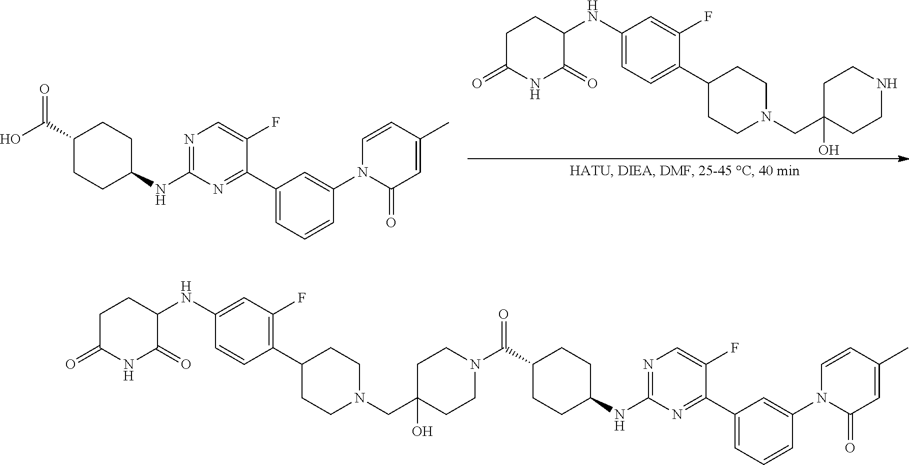 [SMILES: Cc1ccn(-c2cccc(-c3nc(N[C@H]4CC[C@H](C(=O)O)CC4)ncc3F)c2)c(=O)c1 ; O=C1CCC(Nc2ccc(C3CCN(CC4(O)CCNCC4)CC3)c(F)c2)C(=O)N1 ; Cc1ccn(-c2cccc(-c3nc(N[C@H]4CC[C@H](C(=O)N5CCC(O)(CN6CCC(c7ccc(NC8CCC(=O)NC8=O)cc7F)CC6)CC5)CC4)ncc3F)c2)c(=O)c1]

Synthesis of trans-3-[3-fluoro-4-[1-[[1-[4-[[5-fluoro-4-[3-(4-methyl-2-oxo-1-pyridyl)phenyl]pyrimidin-2-yl]amino]cyclohexanecarbonyl]-4-hydroxy-4-piperidyl]methyl]-4-piperidyl]anilino]piperidine-2,6-dione (Compound 472). To a solution of trans-4-[[5-fluoro-4-[3-(4-methyl-2-oxo-1-pyridyl)phenyl]pyrimidin-2-yl]amino]cyclohexanecarboxylic acid (170 mg, 402 μmol, 1.00 eq) in DMF (4 mL) were added DIEA (260 mg, 2.01 mmol, 350 μL, 5.00 eq) and HATU (153 mg, 402 μmol, 1.00 eq) at 25 °C, the mixture was stirred at 25 °C for 10 min. Then 3-[3-fluoro-4-[1-[(4-hydroxy-4-piperidyl)methyl]-4-piperidyl]anilino]piperidine-2,6-dione (236 mg, 443 μmol, 1.10 eq, TFA) was added at 25 °C, the mixture was stirred at 45 °C for 30 min. LCMS showed that the trans-4-[[5-fluoro-4-[3-(4-methyl-2-oxo-1-pyridyl)phenyl]pyrimidin-2-yl]amino]cyclohexanecarboxylic acid was consumed and the desired mass was detected. To the mixture was added H₂O (5 mL) and the mixture was extracted with EtOAc (5 mL*3). The combined organic layers were washed with saturated aqueous NaCl solution (10 mL), dried over Na₂SO₄, filtered and the filtrate was concentrated under reduced pressure to give a residue. The residue was purified by prep-HPLC (column: Phenomenex luna C18 100*40 mm*3 um; mobile phase: [H₂O (0.2% FA)-ACN]; gradient: 15%-45% B over 8.0 min) to give the title compound (Compound 472) (106 mg, 120 μmol, 29.7% yield, 98.47% purity, FA) as a pale yellow solid. LCMS (ES⁺): m/s 823.4 [M+H]⁺

### Example 605. Synthesis of trans-3-((3-fluoro-4-(4-((1-(4-((5-fluoro-4-(3-(4-methyl-2-oxopyridin-1 (2H)-yl)phenyl)pyrimidin-2-yl)amino)cyclohexane-1-carbonyl)piperidin-4-yl)methyl)piperazin-1-yl)phenyl)amino)piperidine-2,6-dione (Compound 471)

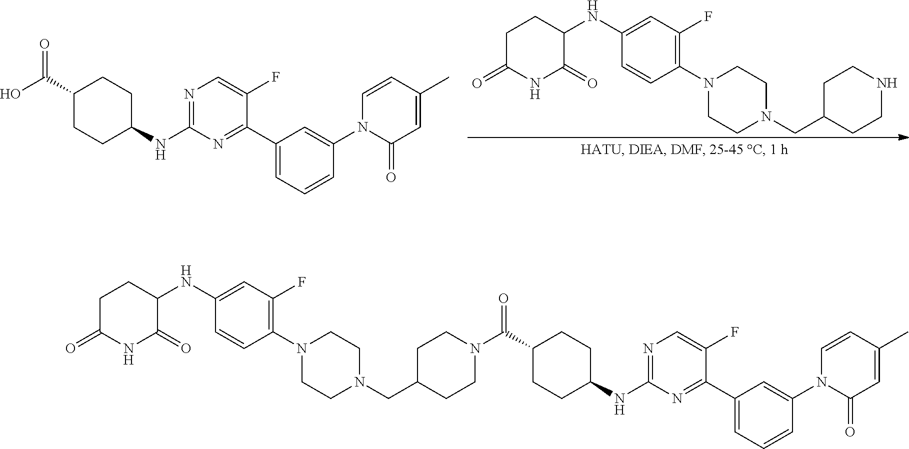

Synthesis of trans-3-((3-fluoro-4-(4-((1-(4-((5-fluoro-4-(3-(4-methyl-2-oxopyridin-1 (2H)-yl)phenyl)pyrimidin-2-yl)amino)cyclohexane-1-carbonyl)piperidin-4-yl)methyl)piperazin-1-yl)phenyl)amino)piperidine-2,6-dione (Compound 471). To a solution of trans-4-((5-fluoro-4-(3-(4-methyl-2-oxopyridin-1 (2H)-yl)phenyl)pyrimidin-2-yl)amino)cyclohexane-1-carboxylic acid (110 mg, 260 μmol, 1.00 eq) in DMF (4 mL) were added HATU (99.0 mg, 260 μmol, 1.00 eq) and DIEA (168 mg, 1.30 mmol, 227 μL, 5.00 eq) at 25 °C, then to the mixture was added 3-((3-fluoro-4-(4-(piperidin-4-ylmethyl)piperazin-1-yl)phenyl)amino)piperidine-2,6-dione (135 mg, 260 μmol, 1.00 eq, TFA) at 25 °C. The resulting mixture was stirred at 25 °C for 1 h. LCMS showed trans-4-((5-fluoro-4-(3-(4-methyl-2-oxopyridin-1 (2H)-yl)phenyl)pyrimidin-2-yl)amino)cyclohexane-1-carboxylic acid was consumed completely and the desired mass was detected. The mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 15%-55% B over 8.0 min) to give the title compound (Compound 471) (25.0 mg, 29.3 μmol, 11.2% yield, 94.6% purity) as a white solid. LCMS (ES⁺): m/z 808.4 [M+H]⁺

### Example 606. Synthesis of trans-3-((3-fluoro-4-(4-((1-(4-((5-fluoro-4-(3-(4-methyl-2-oxopyridin-1 (2H)-yl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 470)

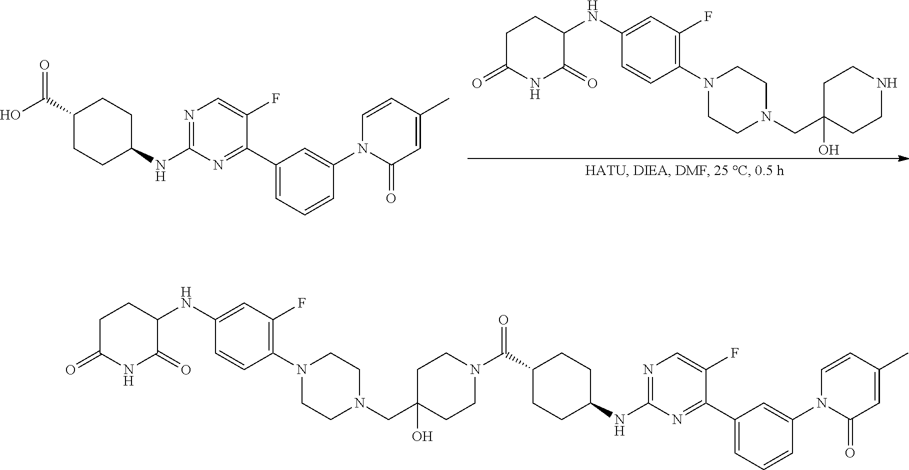

Synthesis of trans-3-((3-fluoro-4-(4-((1-(4-((5-fluoro-4-(3-(4-methyl-2-oxopyridin-1 (2H)-yl)phenyl)pyrimidin-2-yl)amino)cyclohexane-1-carbonyl)-4-hydroxypiperidin-4-yl)methyl)piperazin-1-yl)phenyl)amino)piperidine-2,6-dione (Compound 470). To a stirred solution of trans-4-((5-fluoro-4-(3-(4-methyl-2-oxopyridin-1 (2H)-yl)phenyl)pyrimidin-2-yl)amino)cyclohexane-1-carboxylic acid (130 mg, 307 μmol, 0.800 eq) in DMF (3 mL) were added DIEA (149 mg, 1.15 mmol, 201 μL, 3.00 eq), HATU (175.32 mg, 461.09 μmol, 1.20 eq) and 3-((3-fluoro-4-(4-((4-hydroxypiperidin-4-yl)methyl)piperazin-1-yl)phenyl)amino)piperidine-2,6-dione (205 mg, 384 μmol, 1.00 eq, TFA) at 25 °C. The resulting mixture was stirred at 25 °C for 0.5 h. LCMS showed trans-4-((5-fluoro-4-(3-(4-methyl-2-oxopyridin-1 (2H)-yl)phenyl)pyrimidin-2-yl)amino)cyclohexane-1-carboxylic acid was consumed completely and the desired mass was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 18%-48% B over 8.0 min) to give the title compound (Compound 470) (47.5 mg, 57.2 μmol, 14.9% yield, 99.2% purity) as a pale yellow solid. LCMS (ES⁺): m/z 824.4 [M+H]⁺

### Example 607. Synthesis of 3-(5-(1′-(2-(4-((5-chloro-4-(4′-fluoro-[1,1′-biphenyl]-3-yl)pyrimidin-2-yl)amino)piperidin-1-yl)-2-oxoethyl)-[1,3′-bipiperidin]-4-yl)-1-oxoisoindolin-2-yl)piperidine-2,6-dione (Compound 705)

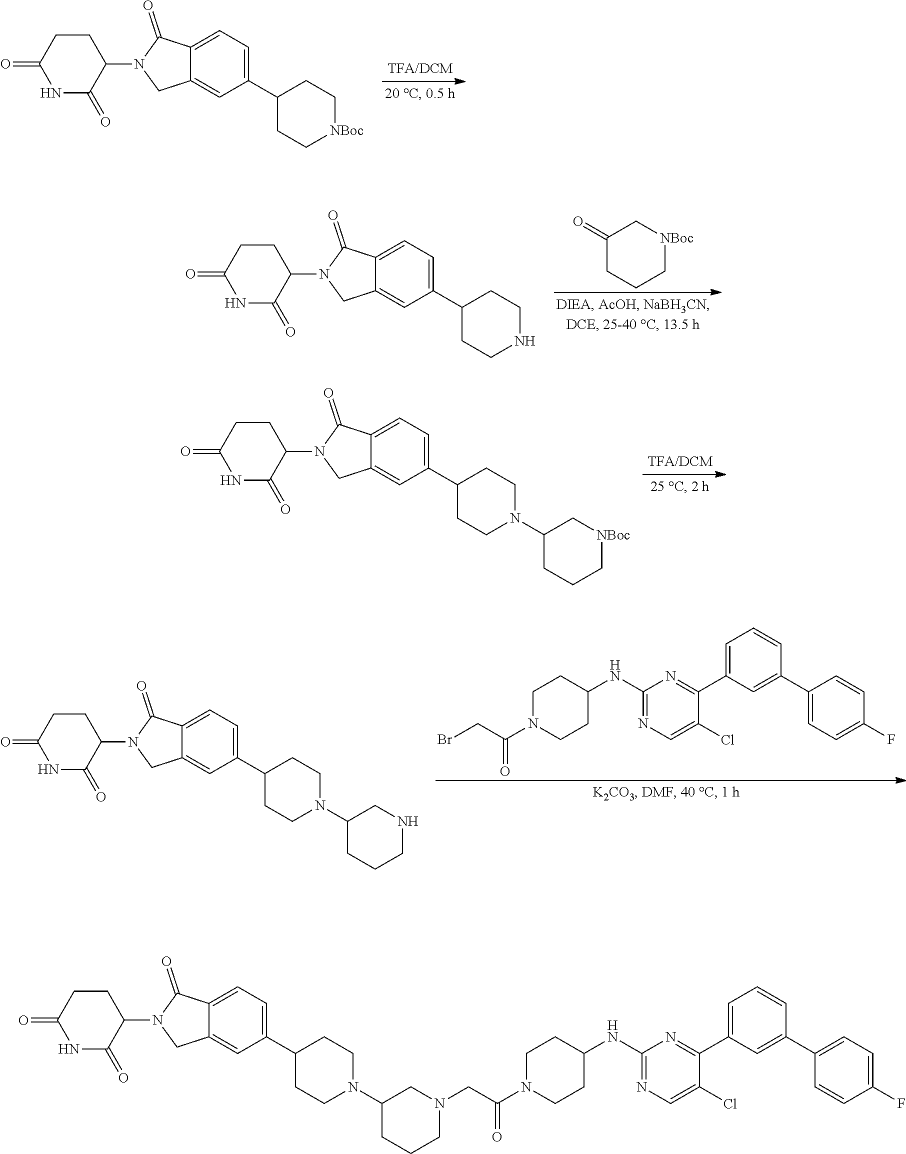

Synthesis of 3-(1-oxo-5-(piperidin-4-yl)isoindolin-2-yl)piperidine-2,6-dione. A solution of tert-butyl 4-(2-(2,6-dioxopiperidin-3-yl)-1-oxoisoindolin-5-yl)piperidine-1-carboxylate (500 mg, 1.17 mmol, 1.00 eq) in DCM (9 mL) and TFA (3 mL) was stirred at 20 °C for 0.5 h. LCMS showed tert-butyl 4-(2-(2,6-dioxopiperidin-3-yl)-1-oxoisoindolin-5-yl)piperidine-1-carboxylate was consumed completely and the desired target MS was detected. The mixture was concentrated under reduced pressure to give the title compound (516 mg, crude, TFA) as a yellow oil. LCMS (ES⁺): m/z 328.1 [M+H]⁺.

Synthesis of tert-butyl 4-(2-(2,6-dioxopiperidin-3-yl)-1-oxoisoindolin-5-yl)-[1,3′-bipiperidine]-1′-carboxylate. To a solution of 3-(1-oxo-5-(piperidin-4-yl)isoindolin-2-yl)piperidine-2,6-dione (516 mg, 1.17 mmol, 1.00 eq, TFA) in DCE (20 mL) was added DIEA (755 mg, 5.85 mmol, 1.02 mL, 5.00 eq) to adjust pH to 8 at 25 °C. The mixture was stirred at 25 °C for 1 h. Then to the reaction mixture was added CH3COOH (7.02 mg, 117 mol, 6.70 μL, 0.100 eq) and tert-butyl 3-oxopiperidine-1-carboxylate (932 mg, 4.68 mmol, 4.00 eq) was added to the mixture at 25 °C, pH was 5 now. The mixture was stirred at 40 °C for 0.5 h. Then to the reaction mixture was added NaBH3CN (294 mg, 4.68 mmol, 4.00 eq) at 25 °C. The resulting mixture was stirred at 40 °C for 12 h. LCMS showed 3-(1-oxo-5-(piperidin-4-yl)isoindolin-2-yl)piperidine-2,6-dione was consumed completely and the desired mass was detected. Water (20 mL) was added to the mixture at 25 °C. The reaction mixture was extracted with DCM (10 mL*4). The organic layers were dried over anhydrous sodium sulfate, filtered and the filtrate was concentrated under reduced re to give a residue. The residue was purified by prep-TLC (SiO₂, Ethyl acetate:Methanol=15:1) to give the title compound (200 mg, 392 μmol, 33.5% yield) as a white solid. LCMS (ES⁺): m/z 511.4 [M+H]⁺.

Synthesis of 3-(5-([1,3′-bipiperidin]-4-yl)-1-oxoisoindolin-2-yl)piperidine-2,6-dione. To a solution of tert-butyl 4-(2-(2,6-dioxopiperidin-3-yl)-1-oxoisoindolin-5-yl)-[1,3′-bipiperidine]-1′-carboxylate (200 mg, 392 μmol, 1.00 eq) in DCM (6 mL) was added TFA (2 mL) at 25 °C. The mixture was stirred at 25 °C for 2 h. LCMS showed tert-butyl 4-(2-(2,6-dioxopiperidin-3-yl)-1-oxoisoindolin-5-yl)-[1,3′-bipiperidine]-1′-carboxylate was consumed completely and the desired mass was detected. The reaction mixture was concentrated under reduced pressure to give the title compound (205 mg, crude, TFA) as a light yellow oil. LCMS (ES⁺): m/z 411.2 [M+H]⁺.

Synthesis of 3-(5-(1′-(2-(4-((5-chloro-4-(4′-fluoro-[1,1′-biphenyl]-3-yl)pyrimidin-2-yl)amino)piperidin-1-yl)-2-oxoethyl)-[1,3′-bipiperidin]-4-yl)-1-oxoisoindolin-2-yl)piperidine-2,6-dione (Compound 705). To a solution of 3-(5-([1,3′-bipiperidin]-4-yl)-1-oxoisoindolin-2-yl)piperidine-2,6-dione (180 mg, 343 μmol, 1.00 eq, TFA) in DMF (7 mL) were added 2-bromo-1-(4-((5-chloro-4-(4′-fluoro-[1,1′-biphenyl]-3-yl)pyrimidin-2-yl)amino)piperidin-1-yl)ethan-1-one (173 mg, 343 μmol, 1.00 eq) and K₂CO₃ (142 mg, 1.03 mmol, 3.00 eq) at 25 °C. The mixture was stirred at 40 °C for 1 h. LCMS showed 3-(5-([1,3′-bipiperidin]-4-yl)-1-oxoisoindolin-2-yl)piperidine-2,6-dione was consumed completely and the desired mass was detected. The reaction mixture was concentrated under reduced pressure to give a residue. The residue was purified by prep-HPLC (column: Phenomenex Luna C18 75*30 mm*3 um; mobile phase: [H₂O (0.1% TFA)-ACN]; gradient: 15%-50% B over 8.0 min) to give the title compound (Compound 705) (50.0 mg, 59.1 μmol, 17.2% yield, 98.5% purity) as a white solid. LCMS (ES⁺): m/z 833.3 [M+H]⁺

### Example 608. Synthesis of trans-2-(1-((1-(4-((2,6-dioxopiperidin-3-yl)amino)-2-fluorophenyl)piperidin-4-yl)methyl)piperidin-4-yl)-N-(4-((5-fluoro-4-(3-(2-oxopyridin-1 (2H)-yl)phenyl)pyrimidin-2-yl)amino)cyclohexyl)acetamide (Compound 704)

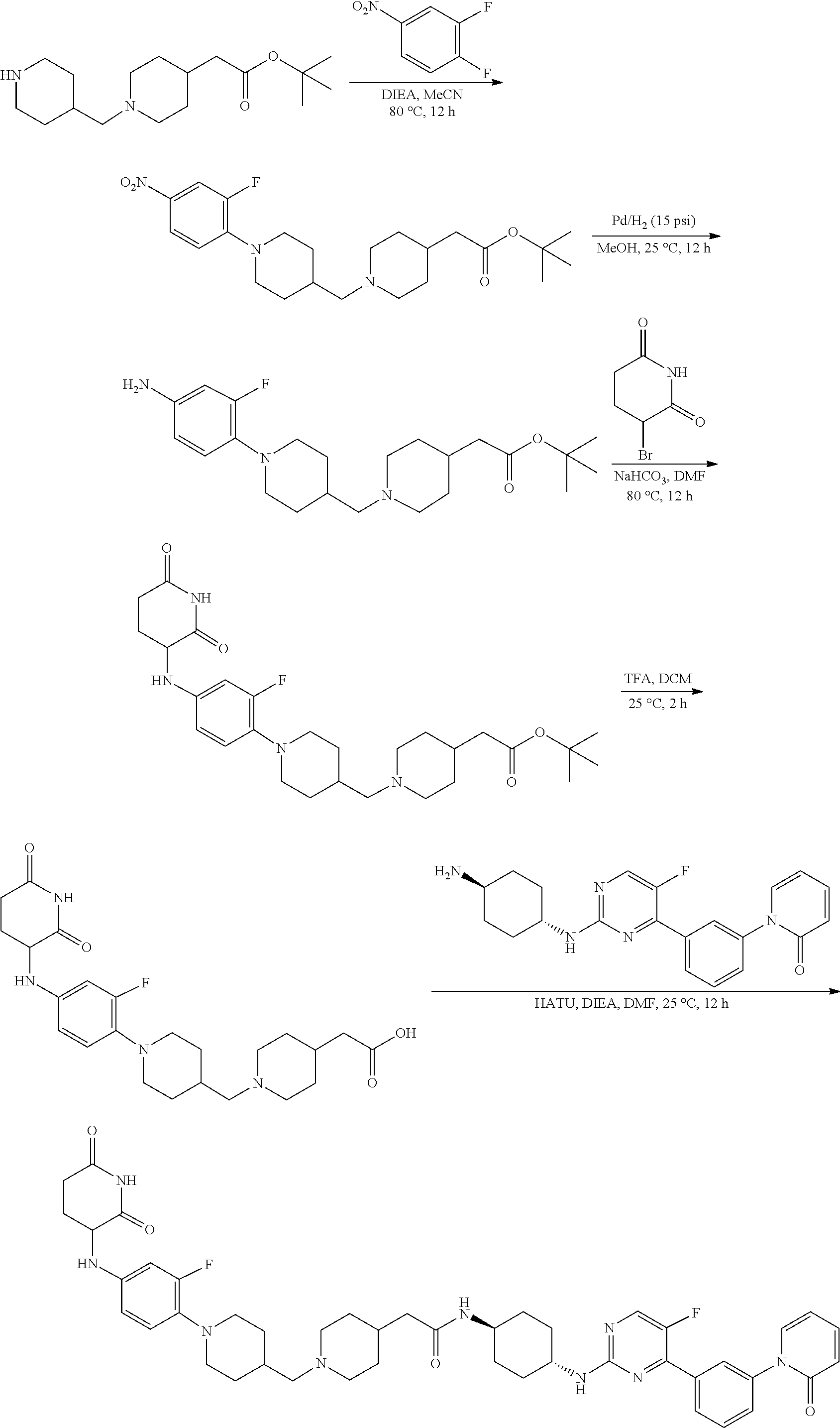

Synthesis of tert-butyl 2-(1-((1-(2-fluoro-4-nitrophenyl)piperidin-4-yl)methyl)piperidin-4-yl)acetate. To a solution of tert-butyl 2-(1-(piperidin-4-ylmethyl)piperidin-4-yl)acetate (2.00 g, 6.75 mmol, 1.00 eq) in MeCN (20.0 mL) was added DIEA (2.62 g, 20.2 mmol, 3.53 mL, 3.00 eq) and 1,2-difluoro-4-nitro-benzene (1.07 g, 6.75 mmol, 746.9 μL, 1.00 eq) at 25 °C. The mixture was stirred at 80 °C for 12 h. LC-MS showed desired mass was detected. The reaction mixture was concentrated under reduced pressure. The crude product was purified by re-crystallization from Petroleum ether (20 mL) at 25 °C to give the title compound (2.56 g, 5.64 mmol, 83.6% yield, 96.0% purity) as a yellow solid. LCMS (ES⁺): m/z 436.3 [M+H]⁺.

Synthesis of tert-butyl 2-(1-((1-(4-amino-2-fluorophenyl)piperidin-4-yl)methyl)piperidin-4-yl)acetate. To a solution of 10% Pd/C (977 mg, 4.59 mmol, 50.0% purity, 1.00 eq) in MeOH (30.0 mL) was added tert-butyl 2-(1-((1-(2-fluoro-4-nitrophenyl)piperidin-4-yl)methyl)piperidin-4-yl)acetate (2.00 g, 4.59 mmol, 1.00 eq) at 25 °C under N₂ atmosphere. The suspension was degassed and purged with H₂ for 3 times. The mixture was stirred under H₂ (15 Psi) at 25 °C for 12 h. LC-MS showed desired mass was detected. The reaction mixture was filtered and the filtrate concentrated under high vacuum. The crude product was purified by re-crystallization from Petroleum ether (30.0 mL) at 25 °C to give the title compound (2.60 g, 4.10 mmol, 89.3% yield, 64.0% purity) as a yellow solid. LCMS (ES⁺): m/z 406.3 [M+H]⁺.

Synthesis of tert-butyl 2-(1-((1-(4-((2,6-dioxopiperidin-3-yl)amino)-2-fluorophenyl)piperidin-4-yl)methyl)piperidin-4-yl)acetate. To a solution of tert-butyl 2-(1-((1-(4-amino-2-fluorophenyl)piperidin-4-yl)methyl)piperidin-4-yl)acetate (2.30 g, 5.67 mmol, 1.00 eq) in DMF (10.0 mL) was added NaHCO₃ (1.43 g, 17.0 mmol, 662 μL, 3.00 eq) and 3-bromopiperidine-2,6-dione (5.44 g, 28.3 mmol, 5.00 eq) at 25 °C. The mixture was stirred at 80 °C for 12 h. LC-MS showed desired mass was detected. The reaction mixture was diluted with water 10 mL and extracted with EtOAc (20 mL*3), filtered and concentrated under reduced pressure. The residue was purified by column chromatography (SiO2, DCM:THF=1:0 to 0:1) to give the title compound (3.90 g, 4.98 mmol, 87.8% yield, 66.0% purity) as a green solid. LCMS (ES⁺): m/z 517.4 [M+H]⁺.

Synthesis of 2-(1-((1-(4-((2,6-dioxopiperidin-3-yl)amino)-2-fluorophenyl)piperidin-4-yl)methyl)piperidin-4-yl)acetic acid. To a solution of tert-butyl 2-(1-((1-(4-((2,6-dioxopiperidin-3-yl)amino)-2-fluorophenyl)piperidin-4-yl)methyl)piperidin-4-yl)acetate (900 mg, 1.74 mmol, 1.00 eq) in DCM (6.0 mL) was added TFA (198 mg, 1.74 mmol, 129 μL, 1.00 eq) at 25 °C. The mixture was stirred at 25 °C for 2 h. LC-MS showed desired mass was detected. The reaction mixture was concentrated under reduced pressure to give the title compound (2.10 g, crude, TFA) as a green oil. LCMS (ES⁺): m/z 461.3 [M+H]⁺.

Synthesis of trans-2-(1-((1-(4-((2,6-dioxopiperidin-3-yl)amino)-2-fluorophenyl)piperidin-4-yl)methyl)piperidin-4-yl)-N-(4-((5-fluoro-4-(3-(2-oxopyridin-1 (2H)-yl)phenyl)pyrimidin-2-yl)amino)cyclohexyl)acetamide (Compound 704). To a solution of 2-(1-((1-(4-((2,6-dioxopiperidin-3-yl)amino)-2-fluorophenyl)piperidin-4-yl)methyl)piperidin-4-yl)acetic acid (100 mg, 217 μmol, 1.00 eq, TFA) in DMF (3.00 mL) was added HATU (123 mg, 325 μmol, 1.50 eq), DIEA (84.1 mg, 651 μmol, 113 μL, 3.00 eq) and trans-1-(3-(2-((4-aminocyclohexyl)amino)-5-fluoropyrimidin-4-yl)phenyl)pyridin-2 (1H)-one (82.3 mg, 217 μmol, 1.00 eq) at 25 °C. The mixture was stirred at 25 °C for 12 h. LC-MS showed desired mass was detected. The reaction mixture was concentrated under reduced pressure to give a residue. The residue was purified by prep-HPLC (column: Phenomenex Luna C18 75*30 mm*3 um; mobile phase: [H₂O (0.2% FA)-ACN]; gradient: 1%-40% B over 8.0 min) to give the title compound (Compound 704) (24.3 mg, 28.0 μmol, 12.9% yield, 98.1% purity, 0.600 FA) as a pink solid. LCMS (ES⁺): m/z 822.4 [M+H]⁺.

### Example 609. Synthesis of (1R,3S,4R)-4-(4-(4-((2,6-dioxopiperidin-3-yl)amino)-2-fluorophenyl)piperidin-1-yl)-3-fluoro-N-((trans)-4-((5-fluoro-4-(3-(2-oxopyridin-1 (2H)-yl)phenyl)pyrimidin-2-yl)amino)cyclohexyl)cyclohexane-1-carboxamide (Compound 694)

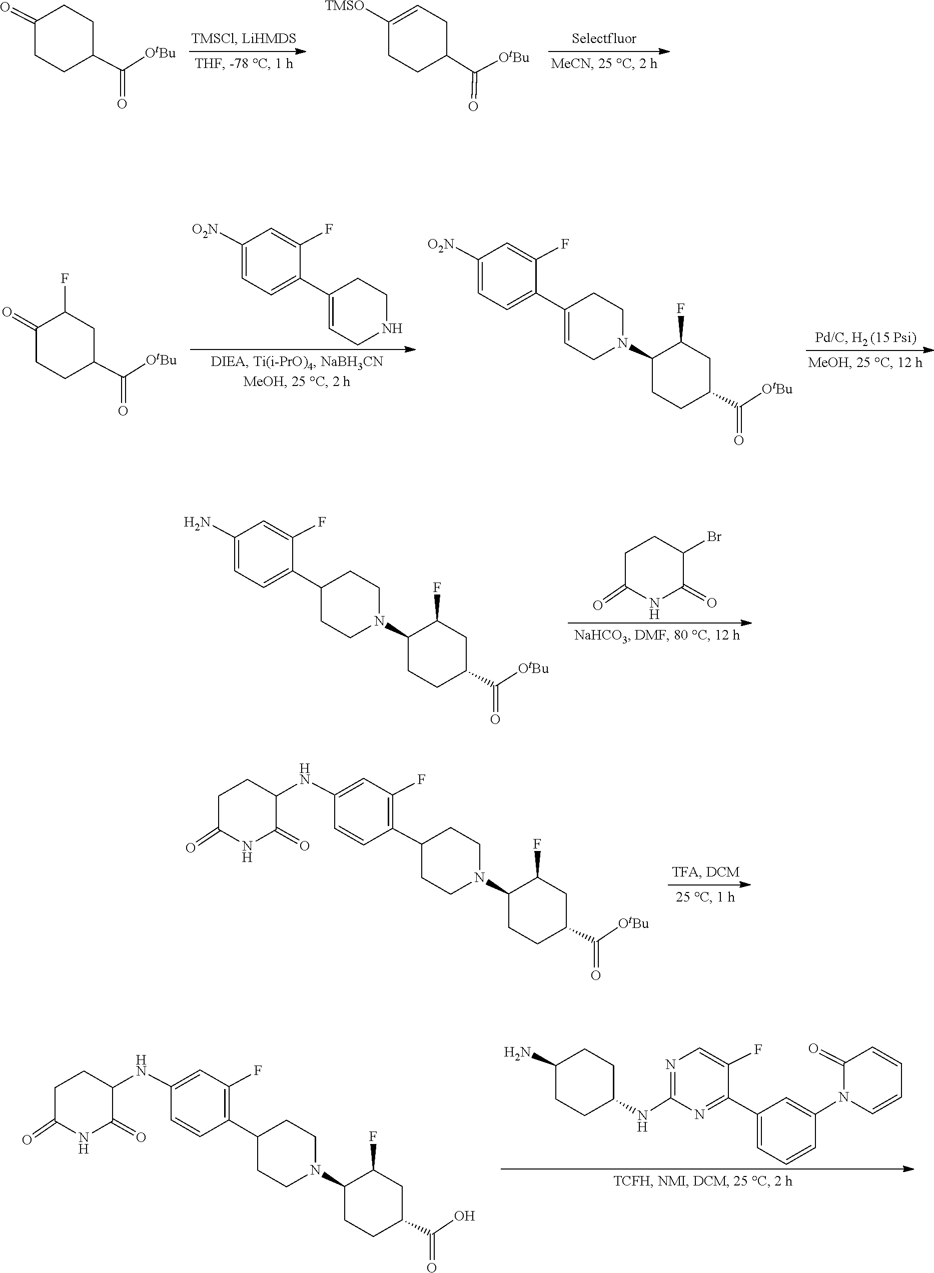

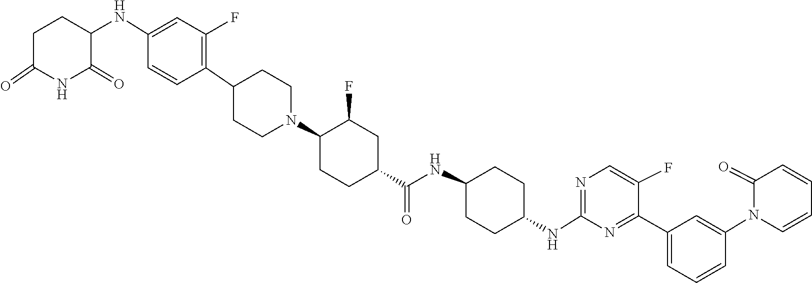 [SMILES: O=C1CCC(Nc2ccc(C3CCN([C@@H]4CC[C@@H](C(=O)N[C@H]5CC[C@H](Nc6ncc(F)c(-c7cccc(-n8ccccc8=O)c7)n6)CC5)C[C@@H]4F)CC3)c(F)c2)C(=O)N1]

Synthesis of tert-butyl 4-((trimethylsilyl)oxy)cyclohex-3-ene-1-carboxylate. A solution of tert-butyl 4-oxocyclohexanecarboxylate (5.00 g, 25.2 mmol, 1.00 eq) in THF (200 mL) was added to LiHMDS (1.00 M, 12.6 mL, 0.500 eq) at −78 °C. After addition, the mixture was stirred at this temperature for 0.5 h under N₂ atmosphere, and then TMSCl (5.48 g, 50.4 mmol, 6.40 mL, 2.00 eq) was added dropwise at −78 °C. The mixture was stirred at −78 °C for 0.5 h. TLC (Petroleum ether:Ethyl acetate=3:1, Rf=0.67) indicated one major new spot was detected. The reaction mixture was quenched by ice water 300 mL at 0 °C, and then extracted with EtOAc (300 mL*3). The combined organic layers were washed with brine (50 mL), dried over Na₂SO₄, filtered and concentrated under reduced pressure. The residue was purified by column chromatography (SiO₂, Petroleum ether:Ethyl acetate=1:0 to 10:1) to give the title compound (50.0 g, crude) as a yellow oil. LCMS (ES⁺): m/z 270.4 [M+H]⁺.

Synthesis of tert-butyl 3-fluoro-4-oxocyclohexane-1-carboxylate. To a solution of tert-butyl 4-trimethylsilyloxycyclohex-3-ene-1-carboxylate (28.0 g, 103 mmol, 1.00 eq) in MeCN (300 mL) was added Select F (55.0 g, 155 mmol, 1.50 eq) at 25 °C. The mixture was stirred at 25 °C for 2 h. TLC (Petroleum ether:Ethyl acetate=3:1, Rf=0.4) indicated one major new spot was detected. The reaction mixture was quenched by ice water 200 mL at 0 °C, and then extracted with EtOAc (500 mL*3). The combined organic layers were washed with brine (100 mL), dried over Na₂SO₄, filtered and concentrated under reduced pressure. The residue was purified by column chromatography (SiO₂, Petroleum ether:Ethyl acetate=1:0 to 3:1) TLC (Petroleum ether:Ethyl acetate=3:1, Rf=0.4) to give the title compound (13.0 g, 60.1 mmol, 58.0% yield) as a yellow oil. LCMS (ES⁺): m/z 216.2 [M+H]⁺.

Synthesis of tert-butyl (1R,3S,4R)-3-fluoro-4-(4-(2-fluoro-4-nitrophenyl)-3,6-dihydropyridin-1 (2H)-yl)cyclohexane-1-carboxylate. To a solution of tert-butyl 3-fluoro-4-oxo-cyclohexanecarboxylate (5.50 g, 25.4 mmol, 1.00 eq) in MeOH (20 mL) was added DIEA (3.29 g, 25.4 mmol, 4.43 mL, 1.00 eq) at 25 °C, The mixture was stirred at 25 °C for 0.5 h, and then add 4-(2-fluoro-4-nitro-phenyl)-1,2,3,6-tetrahydropyridine (5.65 g, 25.4 mmol, 1.00 eq) and Ti(i-PrO)₄ (14.4 g, 50.8 mmol, 15.0 mL, 2.00 eq) at 25 °C, The mixture was stirred at 25 °C for 1 h, and then add NaBH₃CN (4.79 g, 76.3 mmol, 3.00 eq) at 25 °C. The mixture was stirred at 25 °C for 0.5 h. LC-MS showed tert-butyl 3-fluoro-4-oxo-cyclohexanecarboxylate was consumed completely and the desired mass was detected. The reaction was quenched by addition H₂O 80 mL at 25 °C, and then diluted with H₂O 40 mL and then extracted with EtOAc (100 mL*3). The combined organic layers were washed with brine (50 mL*3), dried over Na₂SO₄, filtered and concentrated under reduced pressure to give a residue. The residue was purified by column chromatography (SiO₂, Petroleum ether:Ethyl acetate=5:1 to 1:1) to give the title compound (6.00 g, 13.6 mmol, 53.6% yield, 96.0% purity) as a yellow oil. LCMS (ES⁺): m/z 423.3 [M+H]⁺.

Synthesis of tert-butyl (1R,3S,4R)-4-(4-(4-amino-2-fluorophenyl)piperidin-1-yl)-3-fluorocyclohexane-1-carboxylate. To a solution of tert-butyl 3-fluoro-4-[4-(2-fluoro-4-nitro-phenyl)-3,6-dihydro-2H-pyridin-1-yl]cyclohexanecarboxylate (3.50 g, 8.28 mmol, 1.00 eq) in MeOH (20 mL) was added 10% Pd/C (1.76 g, 8.28 mmol, 50.0% purity, 1.00 eq) at 25 °C under N₂ atmosphere. The suspension was degassed and purged with H₂ for 3 times. The mixture was stirred under H₂ (15 Psi) at 25 °C for 12 h. LC-MS showed tert-butyl 3-fluoro-4-[4-(2-fluoro-4-nitro-phenyl)-3,6-dihydro-2H-pyridin-1-yl]cyclohexanecarboxylate was consumed completely and the desired mass was detected. The reaction mixture was filtered and concentrated under reduced pressure. The residue was purified by column chromatography (SiO₂, Petroleum ether:Ethyl acetate=1:0 to 0:1) to give the title compound (400 mg, 975 μmol, 11.7% yield, 96.1% purity) as a light yellow oil. LCMS (ES⁺): m/z 395.2 [M+H]⁺.

Synthesis of tert-butyl (1R,3S,4R)-4-(4-(4-((2,6-dioxopiperidin-3-yl)amino)-2-fluorophenyl)piperidin-1-yl)-3-fluorocyclohexane-1-carboxylate. To a solution of tert-butyl 4-[4-(4-amino-2-fluoro-phenyl)-1-piperidyl]-3-fluoro-cyclohexanecarboxylate (100 mg, 253 μmol, 1.00 eq) in DMF (2 mL) was added NaHCO₃ (63.8 mg, 760 μmol, 29.5 μL, 3.00 eq) and 3-bromopiperidine-2,6-dione (48.6 mg, 253 μmol, 1.00 eq) at 25 °C. The mixture was stirred at 80 °C for 12 h. LC-MS showed tert-butyl 4-[4-(4-amino-2-fluoro-phenyl)-1-piperidyl]-3-fluoro-cyclohexanecarboxylate was consumed completely and the desired mass was detected. Three in parallel batches were combined and the reaction solution was concentrated under reduced pressure. The residue was purified by column chromatography (SiO₂, Petroleum ether:THF=1:0 to 0:1) to give the title compound (300 mg, 514 μmol, 67.6% yield, 86.6% purity) as a green oil. LCMS (ES⁺): m/z 506.3 [M+H]⁺.

Synthesis of tert-butyl (1R,3S,4R)-4-(4-(4-((2,6-dioxopiperidin-3-yl)amino)-2-fluorophenyl)piperidin-1-yl)-3-fluorocyclohexane-1-carboxylic acid. To a solution of tert-butyl 4-[4-[4-[(2,6-dioxo-3-piperidyl)amino]-2-fluoro-phenyl]-1-piperidyl]-3-fluoro-cyclohexanecarboxylate (200 mg, 395 μmol, 1.00 eq) in DCM (2 mL) was added TFA (15.3 g, 134 mmol, 10.0 mL, 340 eq) at 25 °C. The mixture was stirred at 25 °C for 1 h. LC-MS showed tert-butyl 4-[4-[4-[(2,6-dioxo-3-piperidyl)amino]-2-fluoro-phenyl]-1-piperidyl]-3-fluoro-cyclohexanecarboxylate was consumed completely and the desired mass was detected. The reaction mixture was concentrated under reduced pressure to give the title compound (200 mg, crude, TFA) as a yellow oil. LCMS (ES⁺): m/z 450.3 [M+H]⁺.

Synthesis of (1R,3S,4R)-4-(4-(4-((2,6-dioxopiperidin-3-yl)amino)-2-fluorophenyl)piperidin-1-yl)-3-fluoro-N-((trans)-4-((5-fluoro-4-(3-(2-oxopyridin-1 (2H)-yl)phenyl)pyrimidin-2-yl)amino)cyclohexyl)cyclohexane-1-carboxamide (Compound 694). To a solution of 4-[4-[4-[(2,6-dioxo-3-piperidyl)amino]-2-fluoro-phenyl]-1-piperidyl]-3-fluoro-cyclohexanecarboxylic acid (200 mg, 354 μmol, 1.00 eq, TFA) in DCM (5 mL) was added NMI (87.4 mg, 1.06 mmol, 84.8 μL, 3.00 eq) at 25 °C. The mixture was stirred at 25 °C for 0.5 h, then the trans-1-[3-[2-[(4-aminocyclohexyl)amino]-5-fluoro-pyrimidin-4-yl]phenyl]pyridin-2-one (134 mg, 354 μmol, 1.00 eq) and TCFH (99.5 mg, 354 μmol, 1.00 eq) were added to the reaction solution at 25 °C, The mixture was stirred at 25 °C for 1.5 h. LC-MS showed 4-[4-[4-[(2,6-dioxo-3-piperidyl)amino]-2-fluoro-phenyl]-1-piperidyl]-3-fluoro-cyclohexanecarboxylic acid was consumed completely and the desired mass was detected. The reaction mixture was concentrated under reduced pressure. The residue was purified by prep-HPLC (column: Waters Xbridge Prep OBD C18 150*40 mm*10 um; mobile phase: [H₂O (10 mM NH₄HCO₃)-ACN]; gradient: 35%-65% B over 8.0 min) to give the title compound (Compound 694) (49.7 mg, 57.3 μmol, 16.1% yield, 93.5% purity) as a white solid. LCMS (ES⁺): m/z 811.4 [M+H]⁺.

### Example 610. Synthesis of trans-4-(4-(4-((2,6-dioxopiperidin-3-yl)amino)-2-fluorophenyl)piperidin-1-yl)-N-((trans)-4-((5-fluoro-4-(3-(2-oxopyridin-1 (2H)-yl)phenyl)pyrimidin-2-yl)amino)cyclohexyl)-N-methylcyclohexane-1-carboxamide (Compound 653)

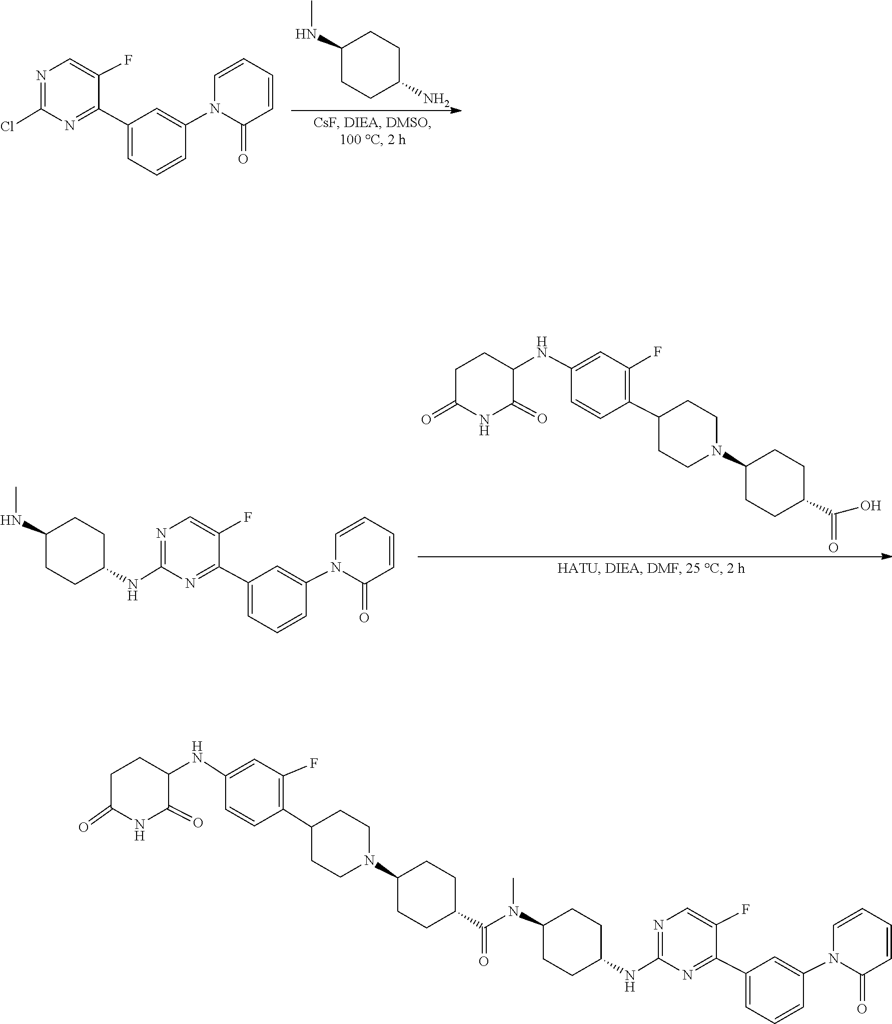

Synthesis of trans-1-(3-(5-fluoro-2-((4-(methylamino)cyclohexyl)amino)pyrimidin-4-yl)phenyl)pyridin-2 (1H)-one. To a solution of N₄-methylcyclohexane-1,4-diamine (169 mg, 1.33 mmol, 2.00 eq) in DMSO (5 mL) was added CsF (100 mg, 662 μmol, 1.00 eq), trans-1-[3-(2-chloro-5-fluoro-pyrimidin-4-yl)phenyl]pyridin-2-one (200 mg, 662 μmol, 1.00 eq) and DIEA (257 mg, 1.99 mmol, 346 μL, 3.00 eq) at 25 °C. The mixture was stirred at 100 °C for 2 h. LC-MS showed the desired mass was detected. The reaction was quenched by addition H₂O 40 mL at 25 °C, and then diluted with H₂O 40 mL and then extracted with EtOAc (70 mL*3). The combined organic layers were washed with brine (50 mL*3), dried over Na₂SO₄, filtered and concentrated under reduced pressure. The residue was purified by prep-HPLC (column: Phenomenex Luna C18 75*30 mm*3 um; mobile phase: [H₂O (0.2% FA)-ACN]; gradient: 5%-45% B over 8.0 min) to give the title compound (150 mg, 373 μmol, 56.3% yield, 98.0% purity) as a pale yellow solid. The product structure was confirmed by 2DNMR. LCMS (ES+): m/z 394.2 [M+H]+.

Synthesis of trans-4-(4-(4-((2,6-dioxopiperidin-3-yl)amino)-2-fluorophenyl)piperidin-1-yl)-N-((trans)-4-((5-fluoro-4-(3-(2-oxopyridin-1 (2H)-yl)phenyl)pyrimidin-2-yl)amino)cyclohexyl)-N-methylcyclohexane-1-carboxamide (Compound 653). To a solution of trans-4-(4-(2-fluoro-4-((6-oxopiperidin-3-yl)amino)phenyl)piperidin-1-yl)cyclohexane-1-carboxylic acid (142 mg, 261 μmol, 7.91e-1 eq, TFA) in DMF (1 mL) was added DIEA (128 mg, 991 μmol, 172 μL, 3.00 eq) at 25 °C. The mixture was stirred at 25 °C for 1 h, then trans-1-[3-[5-fluoro-2-[[4-(methylamino)cyclohexyl]amino]pyrimidin-4-yl]phenyl]pyridin-2-one (130 mg, 330 μmol, 1.00 eq) and HATU (125 mg, 330 μmol, 1.00 eq) added to the reaction solution at 25 °C, the mixture was stirred at 25 °C for 1 h. LC-MS showed the desired mass was detected. The reaction mixture was concentrated under reduced pressure to. The residue was purified by prep-HPLC (column: Waters Xbridge BEH C18 100*30 mm*10 um; mobile phase: [H₂O (10 mM NH₄HCO₃)-ACN]; gradient: 30%-60% B over 8.0 min) to give desired compound as a white solid. The residue was purified by prep-HPLC (column: 3_Phenomenex Luna C18 75*30 mm*3 um; mobile phase: [H₂O (0.2% FA)-ACN]; gradient: 15%-45% B over 8.0 min) to give the title compound (Compound 653) (20.2 mg, 24.2 μmol, 7.35% yield, 99.3% purity, 0.43 FA) as a white solid. LCMS (ES+): m/z 807.4 [M+H]+.

### Example 611. Synthesis of trans-(1r,4R)—N-((1r,4R)-4-((5-chloro-4-(3-(2-oxopyridin-1 (2H)-yl)phenyl)pyrimidin-2-yl)amino)cyclohexyl)-4-(4-(4-((2,6-dioxopiperidin-3-yl)amino)-2-fluorophenyl)piperazin-1-yl)cyclohexane-1-carboxamide (Compound 632)

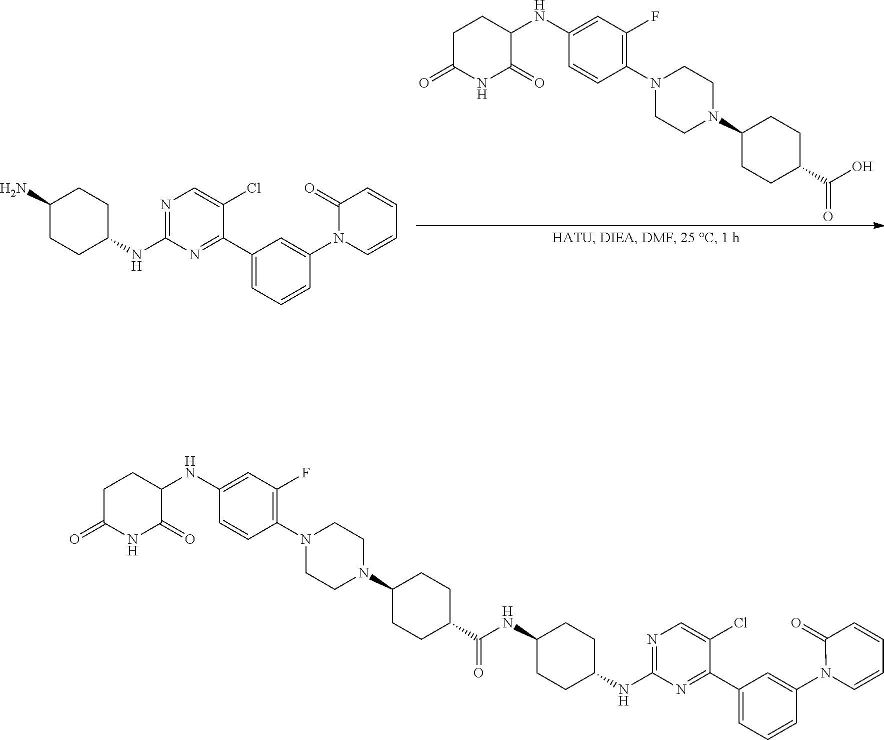 [SMILES: N[C@H]1CC[C@H](Nc2ncc(Cl)c(-c3cccc(-n4ccccc4=O)c3)n2)CC1 ; O=C1CCC(Nc2ccc(N3CCN([C@H]4CC[C@H](C(=O)O)CC4)CC3)c(F)c2)C(=O)N1 ; O=C1CCC(Nc2ccc(N3CCN([C@H]4CC[C@H](C(=O)N[C@H]5CC[C@H](Nc6ncc(Cl)c(-c7cccc(-n8ccccc8=O)c7)n6)CC5)CC4)CC3)c(F)c2)C(=O)N1]

Synthesis of trans-(1r,4R)—N-((1r,4R)-4-((5-chloro-4-(3-(2-oxopyridin-1 (2H)-yl)phenyl)pyrimidin-2-yl)amino)cyclohexyl)-4-(4-(4-((2,6-dioxopiperidin-3-yl)amino)-2-fluorophenyl)piperazin-1-yl)cyclohexane-1-carboxamide (Compound 632). To a solution of trans-4-(4-(4-((2,6-dioxopiperidin-3-yl)amino)-2-fluorophenyl)piperazin-1-yl)cyclohexane-1-carboxylic acid (119 mg, 275 μmol, 1.00 eq) in DMF (4 mL) were added HATU (41.8 mg, 110 μmol, 0.400 eq), DIEA (177 mg, 1.37 mmol, 238 μL, 5.00 eq) and trans-1-(3-(2-((4-aminocyclohexyl)amino)-5-chloropyrimidin-4-yl)phenyl)pyridin-2 (1H)-one (70.0 mg, 137 μmol, 0.500 eq, TFA) at 25 °C. The mixture was stirred at 25 °C for 1 h. LC-MS showed trans-1-(3-(2-((4-aminocyclohexyl)amino)-5-chloropyrimidin-4-yl)phenyl)pyridin-2 (1H)-one was consumed completely and the desired mass was detected. The reaction mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 5%-40% B over 8.0 min) to give the title compound (Compound 632) (39.5 mg, 44.6 μmol, 16.3% yield, 96.8% purity, FA) as a green solid. LCMS (ES⁺): m/z 810.4 [M+H]⁺

### Example 612. Synthesis of trans-N-(trans-4-(4-(4-((2,6-dioxopiperidin-3-yl)amino)-2-fluorophenyl)piperidin-1-yl)cyclohexyl)-4-((5-fluoro-4-(2-oxo-2H-[1,2′-bipyridin]-4′-yl)pyrimidin-2-yl)amino)cyclohexane-1-carboxamide (Compound 696)

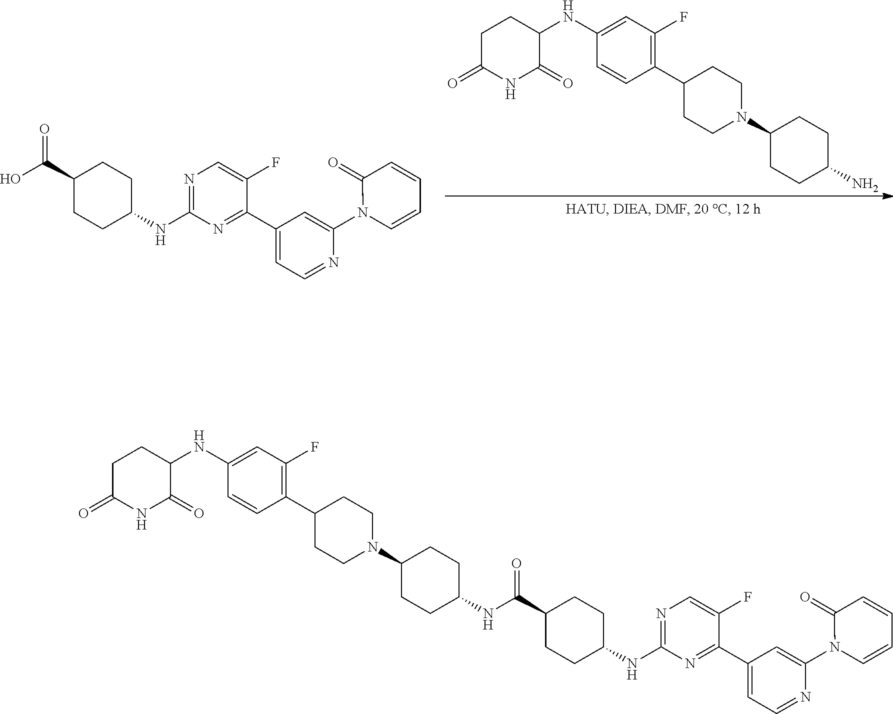 [SMILES: O=c1ccccn1-c1cc(-c2nc(N[C@H]3CC[C@H](C(=O)O)CC3)ncc2F)ccn1 ; N[C@H]1CC[C@H](N2CCC(c3ccc(NC4CCC(=O)NC4=O)cc3F)CC2)CC1 ; O=C1CCC(Nc2ccc(C3CCN([C@H]4CC[C@H](NC(=O)[C@H]5CC[C@H](Nc6ncc(F)c(-c7ccnc(-n8ccccc8=O)c7)n6)CC5)CC4)CC3)c(F)c2)C(=O)N1]

Synthesis of trans-N-(trans-4-(4-(4-((2,6-dioxopiperidin-3-yl)amino)-2-fluorophenyl)piperidin-1-yl)cyclohexyl)-4-((5-fluoro-4-(2-oxo-2H-[1,2′-bipyridin]-4′-yl)pyrimidin-2-yl)amino)cyclohexane-1-carboxamide (Compound 696). To a solution of (trans-4-((5-fluoro-4-(2-oxo-2H-[1,2′-bipyridin]-4′-yl)pyrimidin-2-yl)amino)cyclohexane-1-carboxylic acid (200 mg, 488 μmol, 1.00 eq) in DMF (5 mL) was added 3-[4-[1-(4-aminocyclohexyl)-4-piperidyl]-3-fluoro-anilino]piperidine-2,6-dione (196 mg, 488 μmol, 1.00 eq), DIEA (126 mg, 977 μmol, 170 μL, 2.00 eq) and HATU (371 mg, 977 μmol, 2.00 eq) at 20 °C. The mixture was stirred at 20 °C for 12 h. LC-MS showed the desired mass was detected. The reaction mixture was filtered and the filtrate concentrated under high vacuum. The residue was purified by prep-HPLC (3_Phenomenex Luna C18 75*30 mm*3 um; mobile phase: [H₂O (0.1% TFA)-ACN]; gradient: 10%-40% B over 8.0 min) to give the title compound (Compound 696) (17.0 mg, 21.4 μmol, 4.38% yield, 100% purity) as a green solid. LCMS (ES⁺): m/z 794.4 [M+H]⁺.

### Example 613. Synthesis of trans-N-(trans-4-(4-(4-((2,6-dioxopiperidin-3-yl)amino)-2-fluorophenyl)-3,3-difluoropiperidin-1-yl)cyclohexyl)-4-((5-fluoro-4-(3-(2-oxopyridin-1 (2H)-yl)phenyl)pyrimidin-2-yl)amino)cyclohexane-1-carboxamide (Compound 692)

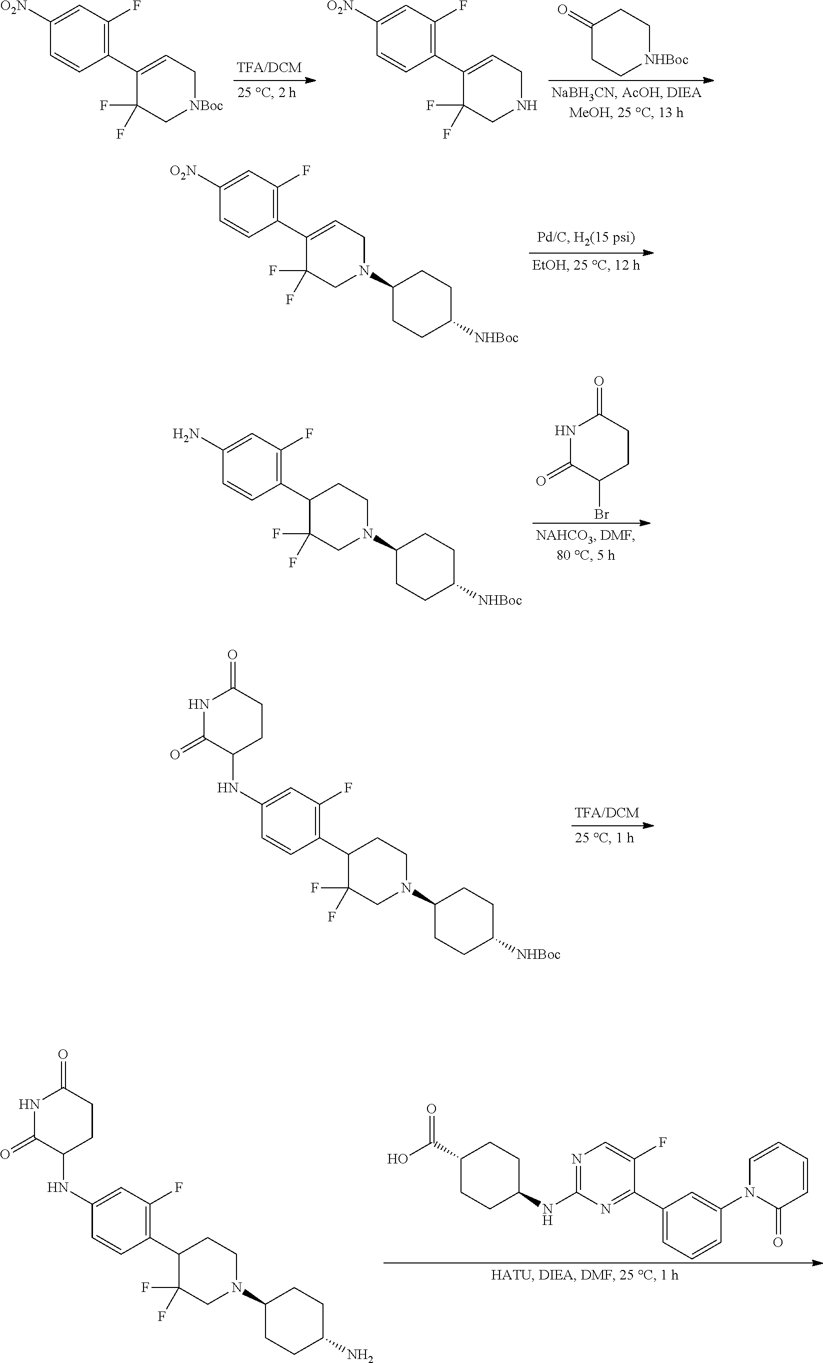

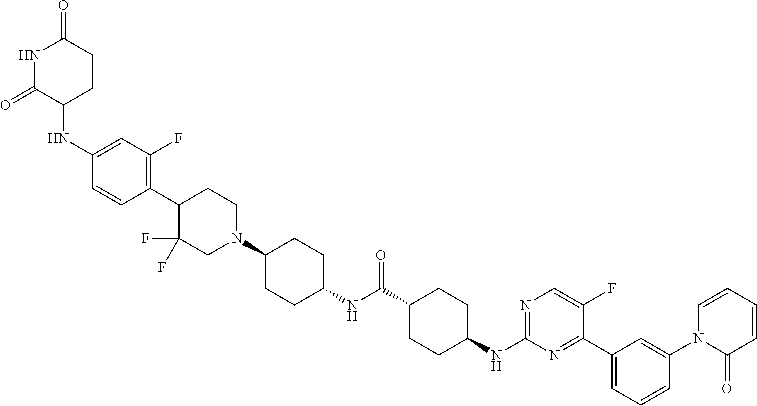 [SMILES: O=C1CCC(Nc2ccc(C3CCN([C@H]4CC[C@H](NC(=O)[C@H]5CC[C@H](Nc6ncc(F)c(-c7cccc(-n8ccccc8=O)c7)n6)CC5)CC4)CC3(F)F)c(F)c2)C(=O)N1]

Synthesis of 3,3-difluoro-4-(2-fluoro-4-nitrophenyl)-1,2,3,6-tetrahydropyridine. To a solution of tert-butyl 3,3-difluoro-4-(2-fluoro-4-nitrophenyl)-3,6-dihydropyridine-1 (2H)-carboxylate (5.00 g, 13.9 mmol, 1.00 eq) in DCM (30 mL) was added TFA (9.21 g, 80.7 mmol, 6 mL, 5.79 eq) at 25 °C. The mixture was stirred at 25 °C for 2 h. LC-MS showed the desired mass was detected. The reaction mixture was concentrated under reduced pressure to give the title compound (5.00 g, crude, TEA) as a brown oil. LCMS (ES⁺): m/z 259.1 [M+H]⁺.

Synthesis of trans-tert-butyl (4-(3,3-difluoro-4-(2-fluoro-4-nitrophenyl)-3,6-dihydropyridin-1 (2H)-yl)cyclohexyl)carbamate. To a solution of 3,3-difluoro-4-(2-fluoro-4-nitrophenyl)-1,2,3,6-tetrahydropyridine (5.00 g, 13.4 mmol, 1.00 eq, TEA) in MeOH (50 mL) was added DIEA (5.21 g, 40.3 mmol, 7.02 mL, 3.00 eq) at 25 °C. The mixture was stirred at 25 °C for 0.5 h. Then AcOH (4.03 g, 67.1 mmol, 3.84 mL, 5.00 eq) and tert-butyl N-(4-oxocyclohexyl)carbamate (11.4 g, 53.7 mmol, 11.4 mL, 4.00 eq) was added at 25 °C. The mixture was stirred at 25 °C for 0.5 h. Then NaBH₃CN (4.22 g, 67.1 mmol, 5.00 eq) at 25 °C. The mixture was stirred at 25 °C for 12 h. LC-MS showed the desired mass was detected. The reaction mixture was concentrated under reduced pressure to give a residue. The reaction mixture was diluted with water 40 mL and extracted with EtOAc (50 mL*3). The combined organic layers dried over Na₂SO₄, filtered and concentrated under reduced pressure. The residue was purified by column chromatography (SiO₂, Petroleum ether:Ethyl acetate=1:0 to 1:1) to give the title compound (800 mg, 1.73 mmol, 12.89% yield, 98.546% purity, rt=2.393 min) was obtained as a white solid. LCMS (ES⁺): m/z 456.2 [M+H]⁺ and cis-tert-butyl N-[4-[3,3-difluoro-4-(2-fluoro-4-nitro-phenyl)-2,6-dihydropyridin-1-yl]cyclohexyl]carbamate (1.2 g, 2.47 mmol, 18.39% yield, 93.773% purity, rt=2.423 min) as a yellow solid. LCMS (ES⁺): m/z 456.3 [M+H]⁺.

Synthesis of trans-tert-butyl (4-(4-(4-amino-2-fluorophenyl)-3,3-difluoropiperidin-1-yl)cyclohexyl)carbamate. To a solution of 10% Pd/C (327 mg, 1.54 mmol, 50% purity, 1.00 eq) in EtOH (10 mL) was added trans-tert-butyl (4-(3,3-difluoro-4-(2-fluoro-4-nitrophenyl)-3,6-dihydropyridin-1 (2H)-yl)cyclohexyl)carbamate (700 mg, 1.54 mmol, 1.00 eq) at 25 °C under N₂ atmosphere. The suspension was degassed and purged with H₂ for 3 times. The mixture was stirred under H₂ (15 Psi) at 25 °C for 12 h. LC-MS showed the desired mass was detected. The reaction mixture was concentrated under reduced pressure to give a residue. The residue was purified by column chromatography (SiO₂, Petroleum ether:Ethyl acetate=1:0 to 0:1) to give the title compound (550 mg, 759 μmol, 49.3% yield, 59.0% purity) as a yellow oil. LCMS (ES⁺): m/z 428.2 [M+H]⁺.

Synthesis of trans-tert-butyl (4-(4-(4-((2,6-dioxopiperidin-3-yl)amino)-2-fluorophenyl)-3,3-difluoropiperidin-1-yl)cyclohexyl)carbamate. To a solution of trans-tert-butyl (4-(4-(4-amino-2-fluorophenyl)-3,3-difluoropiperidin-1-yl)cyclohexyl)carbamate (50.0 mg, 116 μmol, 1.00 eq) in DMF (1 mL) was added NaHCO₃ (39.3 mg, 467 μmol, 18.2 IL, 4.00 eq) and 3-bromopiperidine-2,6-dione (22.4 mg, 116 μmol, 1.00 eq) at 25 °C. The mixture was stirred at 80 °C for 5 h. LC-MS showed the desired mass was detected. The reaction mixture was concentrated under reduced pressure to give a residue. The residue was purified by column chromatography (SiO₂, Petroleum ether:THF=1:0 to 1:1) to give the title compound (330 mg, crude) as a brown oil. LCMS (ES⁺): m/z 539.3 [M+H]⁺.

Synthesis of trans-3-((4-(1-(4-aminocyclohexyl)-3,3-difluoropiperidin-4-yl)-3-fluorophenyl)amino)piperidine-2,6-dione. To a solution of trans-tert-butyl (4-(4-(4-((2,6-dioxopiperidin-3-yl)amino)-2-fluorophenyl)-3,3-difluoropiperidin-1-yl)cyclohexyl)carbamate (200 mg, 371 μmol, 1.00 eq) in DCM (2 mL) was added TFA (1.54 g, 13.4 mmol, 1.00 mL, 36.2 eq) at 25 °C. The mixture was stirred at 25 °C for 1 h. LC-MS showed the desired mass was detected. The reaction mixture was concentrated under reduced pressure to give the title compound (200 mg, crude, TFA) as a brown oil. LCMS (ES⁺): m/z 439.2 [M+H]⁺.

Synthesis of trans-N-(trans-4-(4-(4-((2,6-dioxopiperidin-3-yl)amino)-2-fluorophenyl)-3,3-difluoropiperidin-1-yl)cyclohexyl)-4-((5-fluoro-4-(3-(2-oxopyridin-1 (2H)-yl)phenyl)pyrimidin-2-yl)amino)cyclohexane-1-carboxamide (Compound 692). To a solution of trans-3-((4-(1-(4-aminocyclohexyl)-3,3-difluoropiperidin-4-yl)-3-fluorophenyl)amino)piperidine-2,6-dione (200 mg, 456 μmol, 1.00 eq) in DMF (1 mL) was added DIEA (176 mg, 1.37 mmol, 238 μL, 3.00 eq), trans-4-((5-fluoro-4-(3-(2-oxopyridin-1 (2H)-yl)phenyl)pyrimidin-2-yl)amino)cyclohexane-1-carboxylic acid (186 mg, 456 μmol, 1.00 eq) and HATU (260 mg, 684 μmol, 1.50 eq) at 25 °C. The mixture was stirred at 25 °C for 1 h. LC-MS showed the desired mass was detected. The reaction mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min). The residue was purified by prep-HPLC (column: Phenomenex Luna C18 75*30 mm*3 um; mobile phase: [H₂O (0.2% FA)-ACN]; gradient: 15%-50% B over 8.0 min) to give the title compound (Compound 692) (48.6 mg, 57.4 μmol, 12.5% yield, 97.9% purity) as a white solid. LCMS (ES⁺): m/z 829.4 [M+H]⁺.

### Example 614. Synthesis of trans-3-((3-fluoro-4-(4-((1-(4-((5-fluoro-4-(3-(2-oxopyridin-1 (2H)-yl)phenyl)pyrimidin-2-yl)amino)cyclohexane-1-carbonyl)-4-hydroxypiperidin-4-yl)methyl)piperidin-1-yl)phenyl)amino)piperidine-2,6-dione (Compound 691)

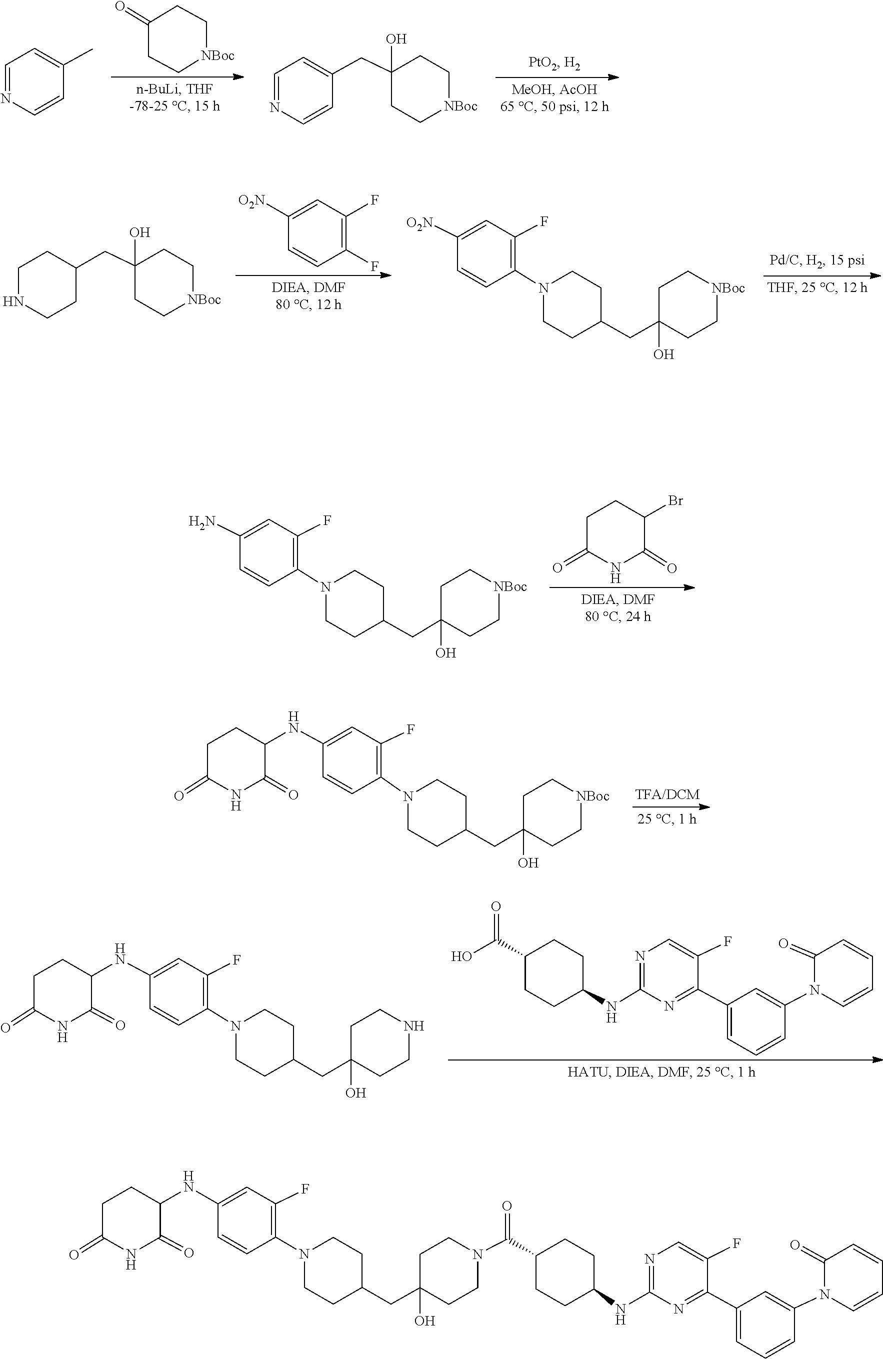

Synthesis of tert-butyl 4-hydroxy-4-(pyridin-4-ylmethyl)piperidine-1-carboxylate. To a solution of 4-methylpyridine (20.0 g, 215 mmol, 21.1 mL, 1.00 eq) in THF (400 mL) was added n-BuLi (2.50 M, 103 mL, 1.20 eq) at −78 °C under N₂ atmosphere. The mixture was stirred at −78 °C for 3 h. Then tert-butyl 4-oxopiperidine-1-carboxylate (42.8 g, 215 mmol, 1.00 eq) in THF (400 mL) was added to the mixture at −78 °C under N₂ atmosphere. The mixture was stirred at 25 °C for 12 h. LCMS showed the desired mass was detected. The reaction mixture was quenched with saturated NH₄Cl solution (800 mL) at 0 °C, then the reaction mixture was extracted with EtOAc (400 mL*3). The organic layers were dried over anhydrous sodium sulfate, filtered and the filtrate was concentrated under reduced pressure to give a residue. The residue was purified by column chromatography (SiO₂, Petroleum ether: Ethyl acetate=1:3 to 0:1) to give the title compound (38.6 g, 132 mmol, 61.5% yield) as a yellow solid. LCMS (ES⁺): m/z 293.0 [M+H]⁺.

Synthesis of tert-butyl 4-hydroxy-4-(piperidin-4-ylmethyl)piperidine-1-carboxylate. PtO₂ (7.50 g, 33.0 mmol, 0.650 eq) was added to MeOH (300 mL) at 25 °C under N₂ atmosphere, then tert-butyl 4-hydroxy-4-(pyridin-4-ylmethyl)piperidine-1-carboxylate (15.0 g, 51.3 mmol, 1.00 eq) and AcOH (15 mL) were added to the mixture at 25 °C, the suspension was degassed and purged with H₂ for three times. The mixture was stirred at 65 °C for 12 h under H₂ (50 psi) atmosphere. LCMS showed tert-butyl 4-hydroxy-4-(pyridin-4-ylmethyl)piperidine-1-carboxylate was consumed completely and the desired mass was detected. The reaction mixture was filtered and the filtrate was concentrated under reduced pressure to give the title compound (15.3 g, crude) as a yellow oil. LCMS (ES⁺): m/z 299.3 [M+H]⁺.

Synthesis of tert-butyl 4-((1-(2-fluoro-4-nitrophenyl)piperidin-4-yl)methyl)-4-hydroxypiperidine-1-carboxylate. To a solution of tert-butyl 4-hydroxy-4-(piperidin-4-ylmethyl)piperidine-1-carboxylate (4.00 g, 13.4 mmol, 1.00 eq) in DMF (100 mL) were added DIEA (5.20 g, 40.2 mmol, 7.00 mL, 3.00 eq) and 1,2-difluoro-4-nitro-benzene (2.13 g, 13.4 mmol, 1.48 mL, 1.00 eq) at 25 °C. The mixture was stirred at 80 °C for 12 h. LCMS showed tert-butyl 4-hydroxy-4-(piperidin-4-ylmethyl)piperidine-1-carboxylate was consumed completely and the desired mass was detected. Water (60 mL) was added to the mixture at 25 °C. The resulting precipitate was collected by filtration. The filter cake was dried under reduced pressure to give the title compound (6.66 g, crude) as a yellow solid. LCMS (ES⁺): m/z 382.1 [M+H−56]⁺.

Synthesis of tert-butyl 4-((1-(4-amino-2-fluorophenyl)piperidin-4-yl)methyl)-4-hydroxypiperidine-1-carboxylate. 10% Pd/C (4.00 g, 50.0% purity) was added to THF (100 mL) at 25 °C under N₂ atmosphere, then tert-butyl 4-((1-(2-fluoro-4-nitrophenyl)piperidin-4-yl)methyl)-4-hydroxypiperidine-1-carboxylate (4.00 g, 9.14 mmol, 1.00 eq) was added to the mixture at 25 °C, the suspension was degassed and purged with H₂ for three times. The mixture was stirred at 25 °C for 12 h under H₂ (15 psi) atmosphere. LCMS showed tert-butyl 4-((1-(2-fluoro-4-nitrophenyl)piperidin-4-yl)methyl)-4-hydroxypiperidine-1-carboxylate was consumed completely and the desired mass was detected. The reaction mixture was filtered and the filtrate was concentrated under reduced pressure to give the title compound (3.70 g, crude) as a yellow oil. LCMS (ES⁺): m/z 408.2 [M+H]⁺.

Synthesis of tert-butyl 4-((1-(4-((2,6-dioxopiperidin-3-yl)amino)-2-fluorophenyl)piperidin-4-yl)methyl)-4-hydroxypiperidine-1-carboxylate. To a solution of tert-butyl 4-((1-(4-amino-2-fluorophenyl)piperidin-4-yl)methyl)-4-hydroxypiperidine-1-carboxylate (3.70 g, 9.08 mmol, 1.00 eq) in DMF (150 mL) were added DIEA (3.52 g, 27.2 mmol, 4.74 mL, 3.00 eq) and 3-bromopiperidine-2,6-dione (8.72 g, 45.4 mmol, 5.00 eq) at 25 °C. The mixture was stirred at 80 °C for 24 h. LCMS showed tert-butyl 4-((1-(4-amino-2-fluorophenyl)piperidin-4-yl)methyl)-4-hydroxypiperidine-1-carboxylate was consumed completely and the desired mass was detected. Water (100 mL) was added to the mixture at 25 °C. Then the reaction mixture was extracted with EtOAc (70 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=1:10 to 0:1) to give the title compound (500 mg, 964 μmol, 10.6% yield) as a green solid. LCMS (ES⁺): m/z 519.4 [M+H]⁺.

Synthesis of 3-((3-fluoro-4-(4-((4-hydroxypiperidin-4-yl)methyl)piperidin-1-yl)phenyl)amino)piperidine-2,6-dione. To a solution of tert-butyl 4-((1-(4-((2,6-dioxopiperidin-3-yl)amino)-2-fluorophenyl)piperidin-4-yl)methyl)-4-hydroxypiperidine-1-carboxylate (200 mg, 386 μmol, 1.00 eq) in DCM (3 mL) was added TFA (1 mL) at 25 °C. The mixture was stirred at 25 °C for 1 h. LCMS showed tert-butyl 4-((1-(4-((2,6-dioxopiperidin-3-yl)amino)-2-fluorophenyl)piperidin-4-yl)methyl)-4-hydroxypiperidine-1-carboxylate was consumed completely and the desired mass was detected. The reaction mixture was concentrated under reduced pressure to give the title compound (200 mg, crude, TFA) as a green oil. LCMS (ES⁺): m/z 419.3 [M+H]⁺.

Synthesis of trans-3-((3-fluoro-4-(4-((1-(4-((5-fluoro-4-(3-(2-oxopyridin-1 (2H)-yl)phenyl)pyrimidin-2-yl)amino)cyclohexane-1-carbonyl)-4-hydroxypiperidin-4-yl)methyl)piperidin-1-yl)phenyl)amino)piperidine-2,6-dione (Compound 691). To a solution of trans-4-[[5-fluoro-4-[3-(2-oxo-1-pyridyl)phenyl]pyrimidin-2-yl]amino]cyclohexanecarboxylic acid (92.1 mg, 225 μmol, 0.600 eq) in DMF (5 mL) were added HATU (42.8 mg, 113 μmol, 0.300 eq), DIEA (146 mg, 1.13 mmol, 196 μL, 3.00 eq) and 3-((3-fluoro-4-(4-((4-hydroxypiperidin-4-yl)methyl)piperidin-1-yl)phenyl)amino)piperidine-2,6-dione (200 mg, 376 μmol, 1.00 eq, TFA) at 25 °C. The mixture was stirred at 25 °C for 1 h. LCMS showed trans-4-[[5-fluoro-4-[3-(2-oxo-1-pyridyl)phenyl]pyrimidin-2-yl]amino]cyclohexanecarboxylic acid was consumed completely and the desired mass was detected. The reaction mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 20%-60% B over 8.0 min) to give the title compound (Compound 691) (58.7 mg, 66.7 μmol, 17.8% yield, 91.9% purity) as a pale yellow solid. LCMS (ES⁺): m/z 809.5 [M+H]⁺.

### Example 615. Synthesis of trans-3-((3-fluoro-4-(1-(1-(4-((5-fluoro-4-(3-(2-oxopyridin-1 (2H)-yl)phenyl)pyrimidin-2-yl)amino)cyclohexane-1-carbonyl)piperidine-4-carbonyl)piperidin-4-yl)phenyl)amino)piperidine-2,6-dione (Compound 669)

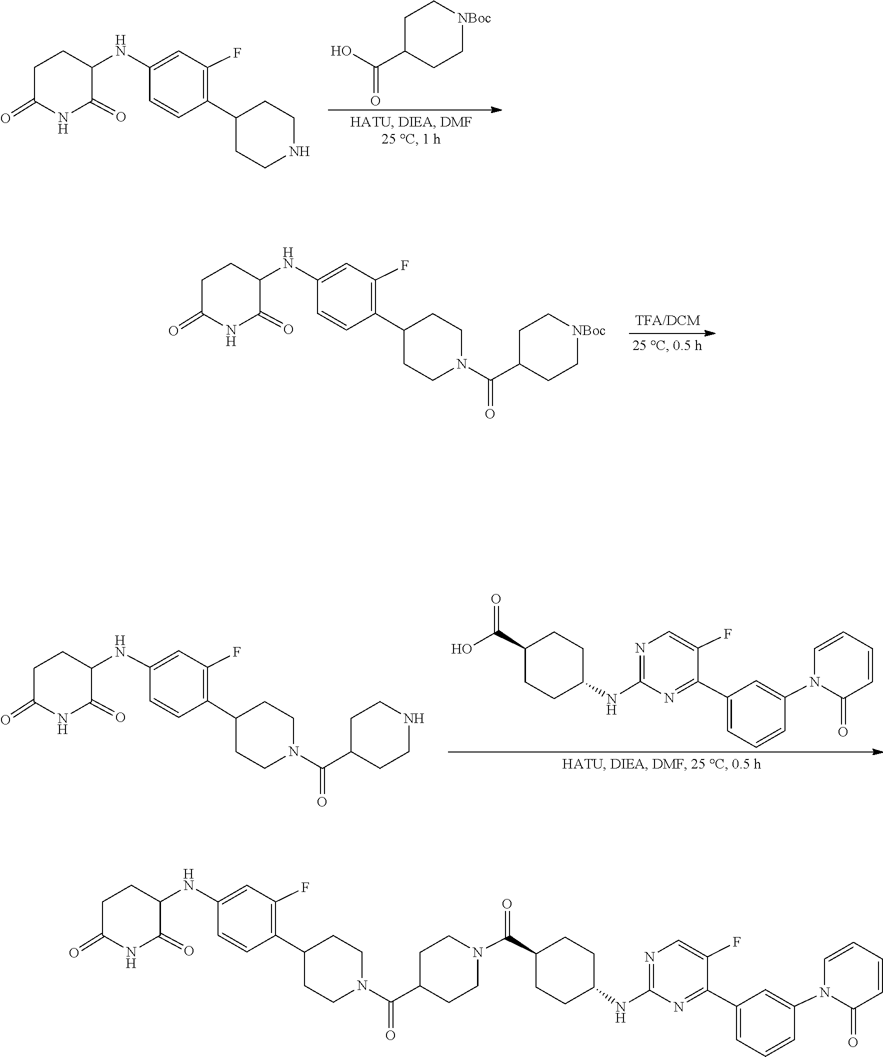 [SMILES: O=C1CCC(Nc2ccc(C3CCNCC3)c(F)c2)C(=O)N1 ; CC(C)(C)OC(=O)N1CCC(C(=O)O)CC1 ; CC(C)(C)OC(=O)N1CCC(C(=O)N2CCC(c3ccc(NC4CCC(=O)NC4=O)cc3F)CC2)CC1 ; O=C1CCC(Nc2ccc(C3CCN(C(=O)C4CCNCC4)CC3)c(F)c2)C(=O)N1 ; O=c1ccccn1-c1cccc(-c2nc(N[C@H]3CC[C@H](C(=O)O)CC3)ncc2F)c1 ; O=C1CCC(Nc2ccc(C3CCN(C(=O)C4CCN(C(=O)[C@H]5CC[C@H](Nc6ncc(F)c(-c7cccc(-n8ccccc8=O)c7)n6)CC5)CC4)CC3)c(F)c2)C(=O)N1]

Synthesis of tert-butyl 4-(4-(4-((2,6-dioxopiperidin-3-yl)amino)-2-fluorophenyl)piperidine-1-carbonyl)piperidine-1-carboxylate. To a solution of 1-tert-butoxycarbonylpiperidine-4-carboxylic acid (226 mg, 986 μmol, 0.800 eq) in DMF (10 mL) were added HATU (469 mg, 1.23 mmol, 1.00 eq), DIEA (797 mg, 6.17 mmol, 1.07 mL, 5.00 eq) and 3-((3-fluoro-4-(piperidin-4-yl)phenyl)amino)piperidine-2,6-dione (517 mg, 1.23 mmol, 1.00 eq, TFA) at 25 °C. The mixture was stirred at 25 °C for 1 h. LC-MS showed 3-((3-fluoro-4-(piperidin-4-yl)phenyl)amino)piperidine-2,6-dione was consumed completely and the desired mass was detected. Water (20 mL) was added to the mixture at 25 °C. Then the reaction mixture was extracted with EtOAc (10 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=1:1 to 1:3) to give the title compound (500 mg, 968 μmol, 78.5% yield) as a blue oil. LCMS (ES⁺): m/z 417.2 [M+H−100]⁺

Synthesis of 3-((3-fluoro-4-(1-(piperidine-4-carbonyl)piperidin-4-yl)phenyl)amino)piperidine-2,6-dione. To a solution of tert-butyl 4-(4-(4-((2,6-dioxopiperidin-3-yl)amino)-2-fluorophenyl)piperidine-1-carbonyl)piperidine-1-carboxylate (200 mg, 387 μmol, 1.00 eq) in DCM (3 mL) was added TFA (1 mL) at 25 °C. The mixture was stirred at 25 °C for 0.5 h. LC-MS showed tert-butyl 4-(4-(4-((2,6-dioxopiperidin-3-yl)amino)-2-fluorophenyl)piperidine-1-carbonyl)piperidine-1-carboxylate was consumed completely and the desired mass was detected. The reaction mixture was concentrated under reduced pressure to give the title compound (205 mg, crude, TFA) as a green oil. LCMS (ES⁺): m/z 417.2 [M+H]⁺

Synthesis of trans-3-((3-fluoro-4-(1-(1-(4-((5-fluoro-4-(3-(2-oxopyridin-1 (2H)-yl)phenyl)pyrimidin-2-yl)amino)cyclohexane-1-carbonyl)piperidine-4-carbonyl)piperidin-4-yl)phenyl)amino)piperidine-2,6-dione (Compound 669). To a solution of trans-4-((5-fluoro-4-(3-(2-oxopyridin-1 (2H)-yl)phenyl)pyrimidin-2-yl)amino)cyclohexane-1-carboxylic acid (78.9 mg, 193 μmol, 0.500 eq) in DMF (6 mL) were added HATU (51.4 mg, 135 μmol, 0.350 eq), DIEA (250 mg, 1.93 mmol, 337 μL, 5.00 eq) and 3-((3-fluoro-4-(1-(piperidine-4-carbonyl)piperidin-4-yl)phenyl)amino)piperidine-2,6-dione (205 mg, 386 μmol, 1.00 eq, TFA) at 25 °C. The mixture was stirred at 25 °C for 0.5 h. LC-MS showed 3-((3-fluoro-4-(1-(piperidine-4-carbonyl)piperidin-4-yl)phenyl)amino)piperidine-2,6-dione was consumed and the desired mass was detected. The reaction mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 25%-55% B over 8.0 min) to give the title compound (Compound 669) (31.7 mg, 38.8 μmol, 10.0% yield, 98.7% purity) as a white solid. LCMS (ES⁺): m/z 807.4 [M+H]⁺

### Example 616. Synthesis of 3-((3-fluoro-4-(1-(((trans)-4-(4-((5-fluoro-4-(3-(2-oxopyridin-1 (2H)-yl)phenyl)pyrimidin-2-yl)amino)piperidine-1-carbonyl)cyclohexyl)methyl)piperidin-4-yl)phenyl)amino)piperidine-2,6-dione (Compound 693)

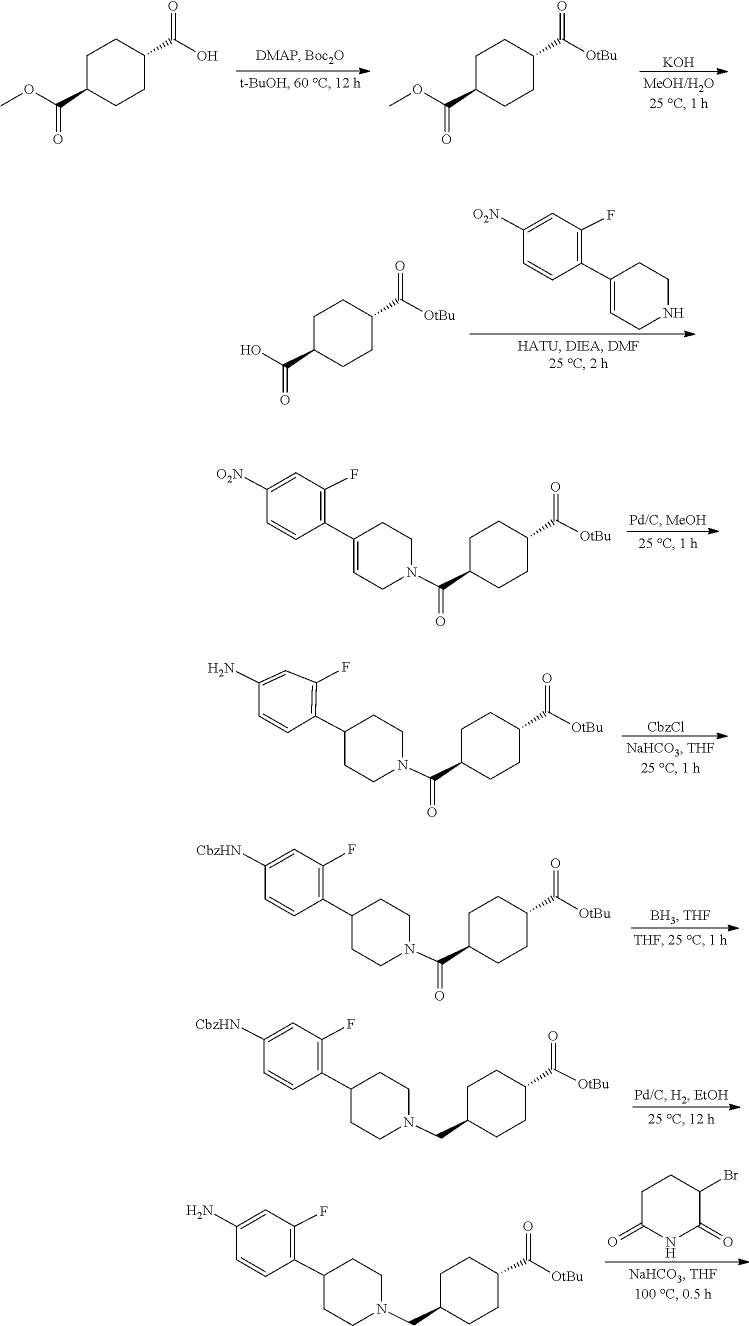

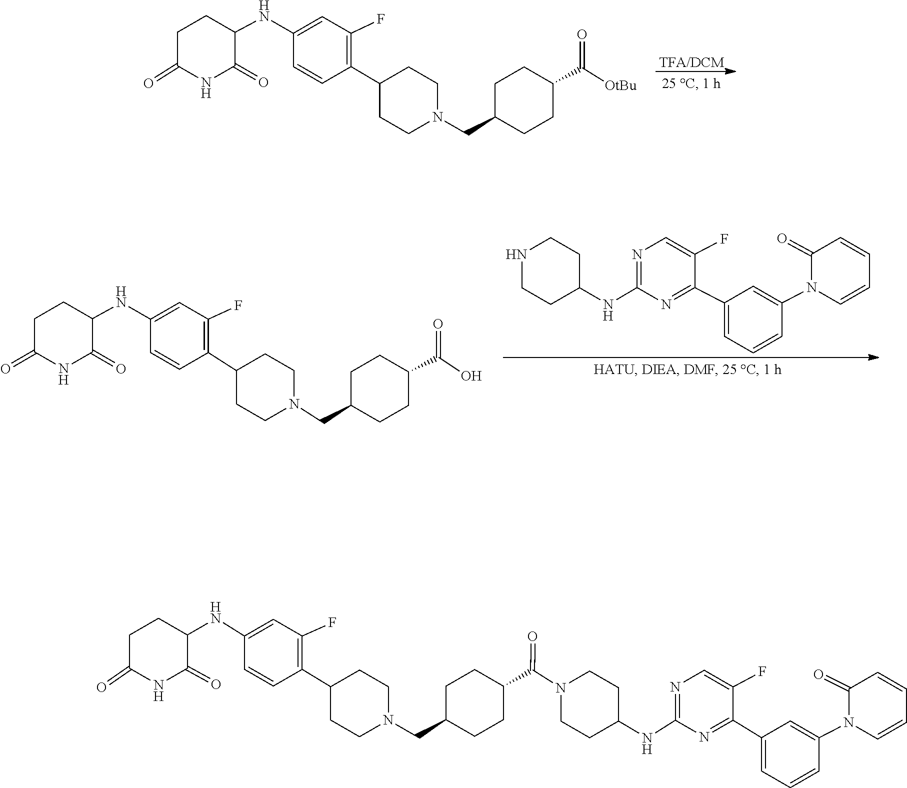 [SMILES: CC(C)(C)OC(=O)[C@H]1CC[C@H](CN2CCC(c3ccc(NC4CCC(=O)NC4=O)cc3F)CC2)CC1 ; "TFA/DCM" ; O=C1CCC(Nc2ccc(C3CCN(C[C@H]4CC[C@H](C(=O)O)CC4)CC3)c(F)c2)C(=O)N1 ; O=c1ccccn1-c1cccc(-c2nc(NC3CCNCC3)ncc2F)c1 ; O=C1CCC(Nc2ccc(C3CCN(C[C@H]4CC[C@H](C(=O)N5CCC(Nc6ncc(F)c(-c7cccc(-n8ccccc8=O)c7)n6)CC5)CC4)CC3)c(F)c2)C(=O)N1]

Synthesis of 1-(tert-butyl) 4-methyl (trans)-cyclohexane-1,4-dicarboxylate. To a solution of DMAP (9.84 g, 80.5 mmol, 0.300 eq) in t-BuOH (1000 mL) was added (Boc)₂₀ (117 g, 537 mmol, 123 mL, 2.00 eq) and trans-4-(methoxycarbonyl)cyclohexane-1-carboxylic acid (50.0 g, 268 mmol, 1.00 eq) at 25 °C. The mixture was stirred at 60 °C for 12 h. TLC indicated one new spot formed. The reaction mixture was quenched by addition H₂O 1000 mL, and extracted with EtOAc 1000 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (40.0 g, crude) as a yellow solid. ¹H NMR (400 MHz, DMSO-d6) δ=3.57 (s, 3H), 2.50-2.10 (m, 2H), 1.92-1.86 (m, 4H), 1.38 (s, 9H), 1.36-1.31 (m, 4H)

Synthesis of trans-4-(tert-butoxycarbonyl)cyclohexane-1-carboxylic acid. To a solution of 1-(tert-butyl) 4-methyl trans-cyclohexane-1,4-dicarboxylate (10.0 g, 41.2 mmol, 1.00 eq) in MeOH (100 mL) and H₂O (60 mL) was added KOH (11.5 g, 206 mmol, 5.00 eq) at 25 °C. The mixture was stirred at 25 °C for 1 h. TLC indicated one new spot formed. The reaction mixture was adjusted with 1 M HCl to pH 6-7. Then the reaction mixture was filtered and filter cake was concentrated under reduced pressure to give the title compound (16.0 g, crude) as a white solid. ¹H NMR (400 MHz, DMSO-d6) δ=12.05 (s, 1H), 2.15-2.13 (m, 2H) 1.91-1.85 (m, 4H), 1.38 (s, 9H), 1.33-1.29 (m, 4H)

Synthesis of tert-butyl trans-4-(4-(2-fluoro-4-nitrophenyl)-1,2,3,6-tetrahydropyridine-1-carbonyl)cyclohexane-1-carboxylate. To a solution of trans-4-(tert-butoxycarbonyl)cyclohexane-1-carboxylic acid (10.0 g, 43.8 mmol, 1.00 eq) and 4-(2-fluoro-4-nitrophenyl)-1,2,3,6-tetrahydropyridine (9.73 g, 37.6 mmol, 8.59° 1 eq, HCl) in DMF (50 mL) was added DIEA (11.3 g, 87.6 mmol, 15.2 mL, 2.00 eq) and HATU (33.3 g, 87.6 mmol, 2.00 eq) at 25 °C. The mixture was stirred at 25 °C for 2 h. LC-MS showed desired mass was detected. The reaction mixture was quenched by addition H₂O 100 mL, and then extracted with EtOAc 300 mL*3. The combined organic layers were filtered and concentrated under reduced pressure. The residue was purified by column chromatography (SiO₂, Petroleum ether:Ethyl acetate=1:0 to 0:1) to give the title compound (10.0 g, 12.7 mmol, 29.0% yield, 55.0% purity) as a yellow solid. LCMS (ES⁺): m/z 433.2 [M+H]⁺.

Synthesis of tert-butyl trans-4-(4-(4-amino-2-fluorophenyl)piperidine-1-carbonyl)cyclohexane-1-carboxylate. To a solution of tert-butyl trans-4-(4-(2-fluoro-4-nitrophenyl)-1,2,3,6-tetrahydropyridine-1-carbonyl)cyclohexane-1-carboxylate (8.00 g, 18.5 mmol, 1.00 eq) in MeOH (100 mL) at 25 °C was added 10% Pd/C (3.00 g, 14.1 mmol, 50.0% purity, 7.62^{e-}1 eq) under N₂ atmosphere. The suspension was degassed and purged with H₂ for 3 times. The mixture was stirred under H₂ (37.3 mg, 18.5 mmol, 1.00 eq) (15 psi) at 25 °C for 1 h. TLC indicated one new spot formed. The reaction mixture was filtered and concentrated under reduced pressure to give the title compound (8.00 g, crude) as a yellow solid. LCMS (ES⁺): m/z 405.3[M+H]⁺.

Synthesis of tert-butyl trans-4-(4-(4-(((benzyloxy)carbonyl)amino)-2-fluorophenyl)piperidine-1-carbonyl)cyclohexane-1-carboxylate. To a solution of tert-butyl tert-butyl trans-4-(4-(4-amino-2-fluorophenyl)piperidine-1-carbonyl)cyclohexane-1-carboxylate (6.00 g, 14.8 mmol, 1.00 eq) in THF (100 mL) was added NaHCO₃ (2.49 g, 29.6 mmol, 1.15 mL, 2.00 eq) and CbzCl (5.06 g, 29.6 mmol, 4.23 mL, 2.00 eq) at 25 °C. The mixture was stirred at 25 °C for 1 h. LC-MS showed the desired mass was detected. Two inparallel batches were combined and the reaction mixture was quenched by addition H₂O 100 mL, and then diluted with H₂O 100 mL and extracted with EtOAc 100 mL*3. The combined organic layers were washed with sat.aq. NaCl (100 mL), dried over Na₂SO₄, filtered and concentrated under reduced pressure. The residue was purified by column chromatography (SiO2, Petroleum ether:Ethyl acetate=1:0 to 0:1) to give the title compound (8.00 g, crude) as a yellow solid. LCMS (ES⁺): m/z 539.3 [M+H]⁺.

Synthesis of tert-butyl trans-4-((4-(4-(((benzyloxy)carbonyl)amino)-2-fluorophenyl)piperidin-1-yl)methyl)cyclohexane-1-carboxylate. To a solution of tert-butyl trans-4-(4-(4-(((benzyloxy)carbonyl)amino)-2-fluorophenyl)piperidine-1-carbonyl)cyclohexane-1-carboxylate (5.00 g, 9.28 mmol, 1.00 eq) in THF (100 mL) was added dropwise BH₃·THF (1 M, 18.5 mL, 2.00 eq) at 0 °C. After addition, the mixture was stirred at 25 °C for 1 h. LC-MS showed the desired mass was detected. The reaction mixture was quenched by addition MeOH 200 mL at 0 °C, and then the reaction mixture was quenched by addition H₂O 100 mL, then extracted with EtOAc 300 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (7.00 g, crude) as a white oil. LCMS (ES⁺): m/z 525.4 [M+H]⁺

Synthesis of tert-butyl trans-4-((4-(4-amino-2-fluorophenyl)piperidin-1-yl)methyl)cyclohexane-1-carboxylate. To a solution of tert-butyl trans-4-((4-(4-(((benzyloxy)carbonyl)amino)-2-fluorophenyl)piperidin-1-yl)methyl)cyclohexane-1-carboxylate (6.00 g, 11.4 mmol, 1.00 eq) in EtOH (100 ml) at 25 °C was added 10% Pd/C (4.00 g, 18.7 mmol, 50.0% purity, 1.64 eq) under N₂ atmosphere. The suspension was degassed and purged with H₂ for 3 times. The mixture was stirred under H₂ (23.1 mg, 11.4 mmol, 1.00 eq) (15 psi) at 25 °C for 12 h. LC-MS showed the desired mass was detected. The reaction mixture was filtered and concentrated under reduced pressure to give the title compound (7.00 g, crude) as a colorless oil. LCMS (ES⁺): m/z 391.3 [M+H]⁺

Synthesis of tert-butyl-trans-4-((4-(4-((2,6-dioxopiperidin-3-yl)amino)-2-fluorophenyl)piperidin-1-yl)methyl)cyclohexane-1-carboxylate. To a solution of tert-butyl trans-4-((4-(4-amino-2-fluorophenyl)piperidin-1-yl)methyl)cyclohexane-1-carboxylate (6.00 g, 15.3 mmol, 1.00 eq) and 3-bromopiperidine-2,6-dione (2.95 g, 15.3 mmol, 1.00 eq) in DMF (50 mL) was added NaHCO₃ (10.3 g, 122 mmol, 4.78 mL, 8.00 eq) at 25 °C. The mixture was stirred at 100 °C for 0.5 h. LC-MS showed the desired mass was detected. The reaction mixture was quenched by addition H₂O 100 mL, and then extracted with EtOAc 150 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (700 mg, 1.20 mmol, 7.81% yield, 86.0% purity) as a green solid. LCMS (ES⁺): m/z 502.3 [M+H]⁺

Synthesis of trans-4-((4-(4-((2,6-dioxopiperidin-3-yl)amino)-2-fluorophenyl)piperidin-1-yl)methyl)cyclohexane-1-carboxylic acid. To a solution of tert-butyl trans-4-((4-(4-((2,6-dioxopiperidin-3-yl)amino)-2-fluorophenyl)piperidin-1-yl)methyl)cyclohexane-1-carboxylate (100 mg, 199 μmol, 1.00 eq) in DCM (3 mL) was added TFA (1.54 g, 13.4 mmol, 1.00 mL, 67.5 eq) at 25 °C. The mixture was stirred at 25 °C for 1 h. LC-MS showed the desired mass was detected. The reaction mixture was concentrated under reduced pressure to give the title compound (100 mg, crude, TFA) as a black oil. LCMS (ES⁺): m/z 446.2 [M+H]⁺.

Synthesis of 3-((3-fluoro-4-(1-((trans-4-(4-((5-fluoro-4-(3-(2-oxopyridin-1 (2H)-yl)phenyl)pyrimidin-2-yl)amino)piperidine-1-carbonyl)cyclohexyl)methyl)piperidin-4-yl)phenyl)amino)piperidine-2,6-dione (Compound 693). To a solution of trans-4-((4-(4-((2,6-dioxopiperidin-3-yl)amino)-2-fluorophenyl)piperidin-1-yl)methyl)cyclohexane-1-carboxylic acid (80.0 mg, 142 μmol, 1.00 eq, TFA) and 1-(3-(5-fluoro-2-(piperidin-4-ylamino)pyrimidin-4-yl)phenyl)pyridin-2 (1H)-one (57.4 mg, 142 μmol, 1.00 eq, HCl) in DMF (2 mL) was added DIEA (36.9 mg, 285 μmol, 49.8 μL, 2.00 eq) and HATU (108 mg, 285 μmol, 2.00 eq) at 25 °C. The mixture was stirred at 25 °C for 1 h. LC-MS showed the desired mass was detected. The reaction mixture was concentrated under reduced pressure. The residue was purified by prep-HPLC (column: Phenomenex luna C18 100*40 mm*5 um; mobile phase: [H₂O (0.2% FA)-ACN]; gradient: 5%-45% B over 8.0 min) to give the title compound (Compound 693) (24.6 mg, 28.7 μmol, 20.0% yield, 93.6% purity, 0.200 FA) as a pale yellow solid. LCMS (ES⁺): m/z 793.4 [M+H]⁺

### Example 617. Synthesis of trans-(1r,4R)-4-((4-(3-(2,6-dioxopiperidin-3-yl)-1-methyl-1H-indazol-6-yl)piperidin-1-yl)methyl)-N-((1r,4R)-4-((5-fluoro-4-(3-(2-oxopyridin-1 (2H)-yl)phenyl)pyrimidin-2-yl)amino)cyclohexyl)cyclohexane-1-carboxamide (Compound 535)

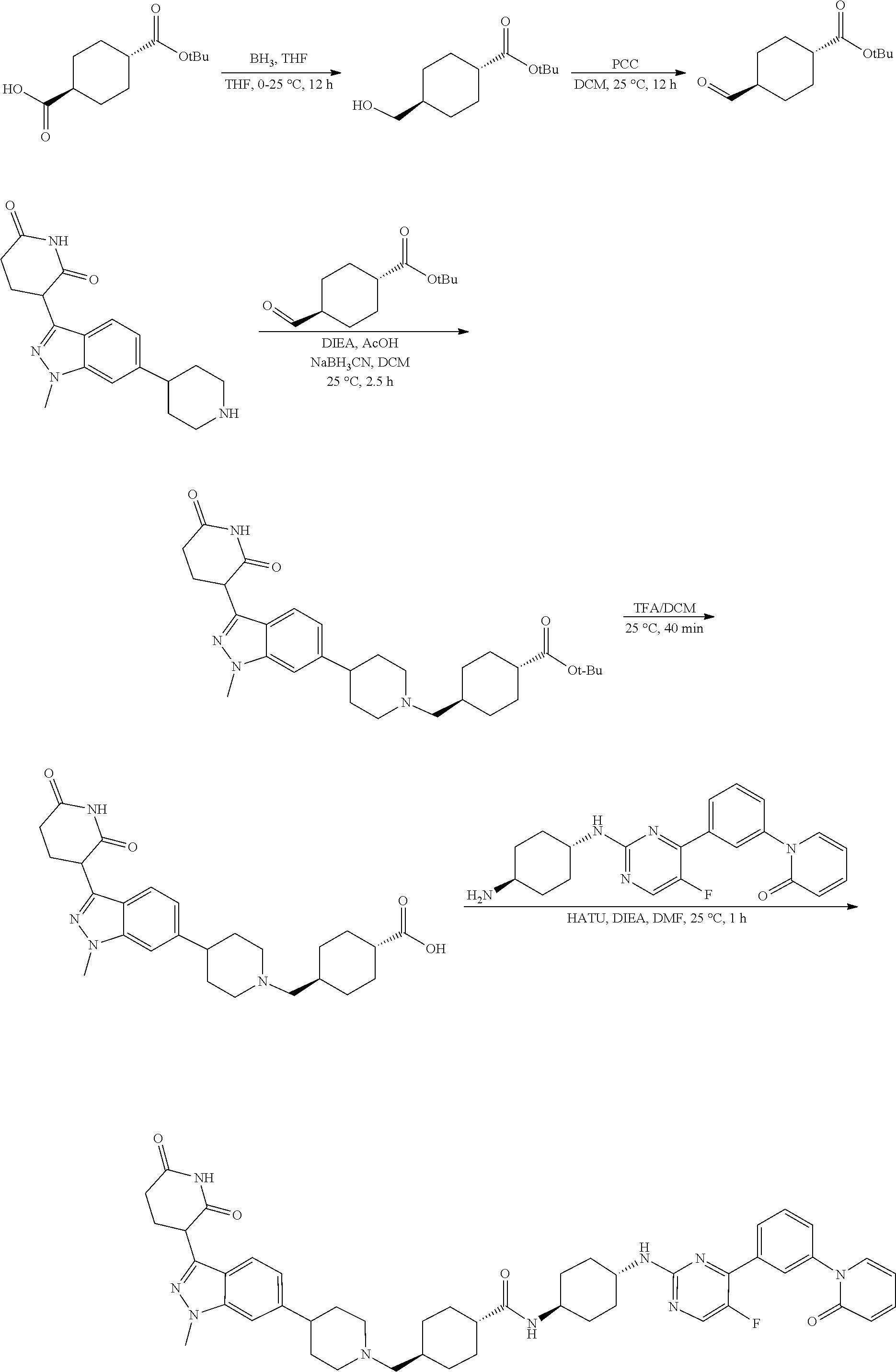

Synthesis of trans-tert-butyl 4-(hydroxymethyl)cyclohexane-1-carboxylate. To a solution of trans-4-(tert-butoxycarbonyl)cyclohexane-1-carboxylic acid (8.00 g, 35.0 mmol, 1.00 eq) in THF (80 mL) was added BH₃-THF (1.00 M, 48.0 mL, 1.37 eq) under N₂ atmosphere at 0 °C. The mixture was stirred at 25 °C for 12 h. TLC (SiO₂, Petroleum ether:Ethyl acetate=3:1) showed trans-4-(tert-butoxycarbonyl)cyclohexane-1-carboxylic acid was consumed completely, and one new spot formed. Water (40 mL) was added to the reaction mixture to quench the reaction at 0 °C. Then the mixture was stirred at 0 °C for 0.5 h. Then the reaction mixture was extracted with EtOAc (30 mL*3). The organic layers were dried over anhydrous sodium sulfate, filtered and the filtrate was concentrated under reduced pressure to give the title compound (6.50 g, crude) as a white clear oil. The product couldn't be detected by LCMS. It was confirmed by ¹H NMR (400 MHz, CDCl₃) δ=3.66-3.53 (m, 1H), 3.49 (d, J=4.4 Hz, 1H), 2.45 (dt, J=9.8, 4.9 Hz, 1H), 1.96 (ddd, J=16.0, 9.4, 4.0 Hz, 2H), 1.62-1.47 (m, 4H), 1.42 (dd, J=4.6, 1.0 Hz, 9H), 1.32-1.21 (m, 2H), 1.02-0.81 (m, 1H).

Synthesis of trans-tert-butyl 4-formylcyclohexane-1-carboxylate. To a solution of trans-tert-butyl 4-(hydroxymethyl)cyclohexane-1-carboxylate (4.00 g, 18.7 mmol, 1.00 eq) in DCM (80 mL) was added PCC (8.05 g, 37.3 mmol, 2.00 eq) at 25 °C. The mixture was stirred at 25 °C for 12 h. TLC (SiO₂, Petroleum ether:Ethyl acetate=3:1) showed trans-tert-butyl 4-(hydroxymethyl)cyclohexane-1-carboxylate was consumed completely, and one new spot formed. The reaction mixture filtered and the filtrate was concentrated under reduced pressure to give a residue. Water (100 mL) was added to the mixture at 25 °C. Then the reaction mixture was extracted with EtOAc (50 mL*3). The organic layers were dried over anhydrous sodium sulfate, filtered and the filtrate was concentrated under reduced pressure to give the title compound (2.00 g, crude) as a brown oil. The product couldn't be detected by LCMS. It was confirmed by ¹H NMR (400 MHz, CDCl₃) δ=9.66-9.61 (m, 1H), 2.32 (ddd, J=8.4, 4.0, 2.4 Hz, 1H), 2.25-2.09 (m, 1H), 2.07-2.02 (m, 1H), 2.00-1.82 (m, 2H), 1.73-1.66 (m, 4H), 1.43-1.41 (m, 9H), 1.29-1.23 (m, 1H).

Synthesis of trans-tert-butyl 4-((4-(3-(2,6-dioxopiperidin-3-yl)-1-methyl-1H-indazol-6-yl)piperidin-1-yl)methyl)cyclohexane-1-carboxylate. To a solution of 3-(1-methyl-6-(piperidin-4-yl)-1H-indazol-3-yl)piperidine-2,6-dione (800 mg, 1.82 mmol, 0.297 eq, TFA) in DCM (30 mL) was added DIEA at 25 °C to adjust pH to 8. The mixture was stirred at 25 °C for 0.5 h. Then trans-tert-butyl 4-formylcyclohexane-1-carboxylate (1.30 g, 6.12 mmol, 1.00 eq) and AcOH (368 mg, 6.12 mmol, 351 μL, 1.00 eq) were added to the reaction mixture at 25 °C. The mixture was stirred at 25 °C for 1 h, pH was 5 now. Then NaBH₃CN (1.92 g, 30.6 mmol, 5.00 eq) was added to the reaction mixture at 25 °C. The mixture was stirred at 25 °C for 1 h. LCMS showed 3-(1-methyl-6-(piperidin-4-yl)-1H-indazol-3-yl)piperidine-2,6-dione was consumed completely and the desired mass was detected. The reaction mixture was concentrated under reduced pressure to give a residue. The residue was purified by column chromatography (SiO₂, Petroleum ether:Ethyl acetate=5:1 to 3:1) to give the title compound (2.00 g, 2.33 mmol, 38.1% yield, 60.9% purity) as a green oil. LCMS (ES⁺): m/z 523.3 [M+H]⁺.

Synthesis of trans-4-((4-(3-(2,6-dioxopiperidin-3-yl)-1-methyl-1H-indazol-6-yl)piperidin-1-yl)methyl)cyclohexane-1-carboxylic acid. To a solution of trans-tert-butyl 4-((4-(3-(2,6-dioxopiperidin-3-yl)-1-methyl-1H-indazol-6-yl)piperidin-1-yl)methyl)cyclohexane-1-carboxylate (430 mg, 823 μmol, 1.00 eq) in DCM (6 mL) was added TFA (2 mL) at 25 °C. The mixture was stirred at 25 °C for 40 min. LCMS showed trans-tert-butyl 4-((4-(3-(2,6-dioxopiperidin-3-yl)-1-methyl-1H-indazol-6-yl)piperidin-1-yl)methyl)cyclohexane-1-carboxylate was consumed completely and the desired mass was detected. The reaction mixture was concentrated under reduced pressure to give the title compound (380 mg, crude) as a green oil. LCMS (ES⁺): m/z 467.3 [M+H]⁺.

Synthesis of trans-(1r,4R)-4-((4-(3-(2,6-dioxopiperidin-3-yl)-1-methyl-1H-indazol-6-yl)piperidin-1-yl)methyl)-N-((1r,4R)-4-((5-fluoro-4-(3-(2-oxopyridin-1 (2H)-yl)phenyl)pyrimidin-2-yl)amino)cyclohexyl)cyclohexane-1-carboxamide (Compound 535). To a solution of trans-4-((4-(3-(2,6-dioxopiperidin-3-yl)-1-methyl-1H-indazol-6-yl)piperidin-1-yl)methyl)cyclohexane-1-carboxylic acid (380 mg, 814 μmol, 1.00 eq) in DMF (4 mL) were added DIEA (525 mg, 4.07 mmol, 710 μL, 5.00 eq) and HATU (124 mg, 326 μmol, 0.400 eq) at 25 °C. Then trans-1-(3-(2-((4-aminocyclohexyl)amino)-5-fluoropyrimidin-4-yl)phenyl)pyridin-2 (1H)-one (201 mg, 407 μmol, 0.500 eq, TFA) was added to the reaction mixture at 25 °C. The mixture was stirred at 25 °C for 1 h. LCMS showed trans-4-((4-(3-(2,6-dioxopiperidin-3-yl)-1-methyl-1H-indazol-6-yl)piperidin-1-yl)methyl)cyclohexane-1-carboxylic acid was consumed completely and the desired mass was detected. The reaction mixture was filtered and the filtrate was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 40%-70% B over 8.0 min) to give the title compound (Compound 535) (55.4 mg, 63.0 μmol, 7.74% yield, 94.2% purity) as a pale yellow solid. LCMS (ES⁺): m/z 828.5 [M+H]⁺.

### Example 618. Synthesis of trans-(1R,4r)-4-(4-(4-(((R)-2,6-dioxopiperidin-3-yl)amino)-2-fluorophenyl)piperazin-1-yl)cyclohexyl 4-((5-fluoro-4-(3-(2-oxopyridin-1 (2H)-yl)phenyl)pyrimidin-2-yl)amino)piperidine-1-carboxylate (Compound 699)

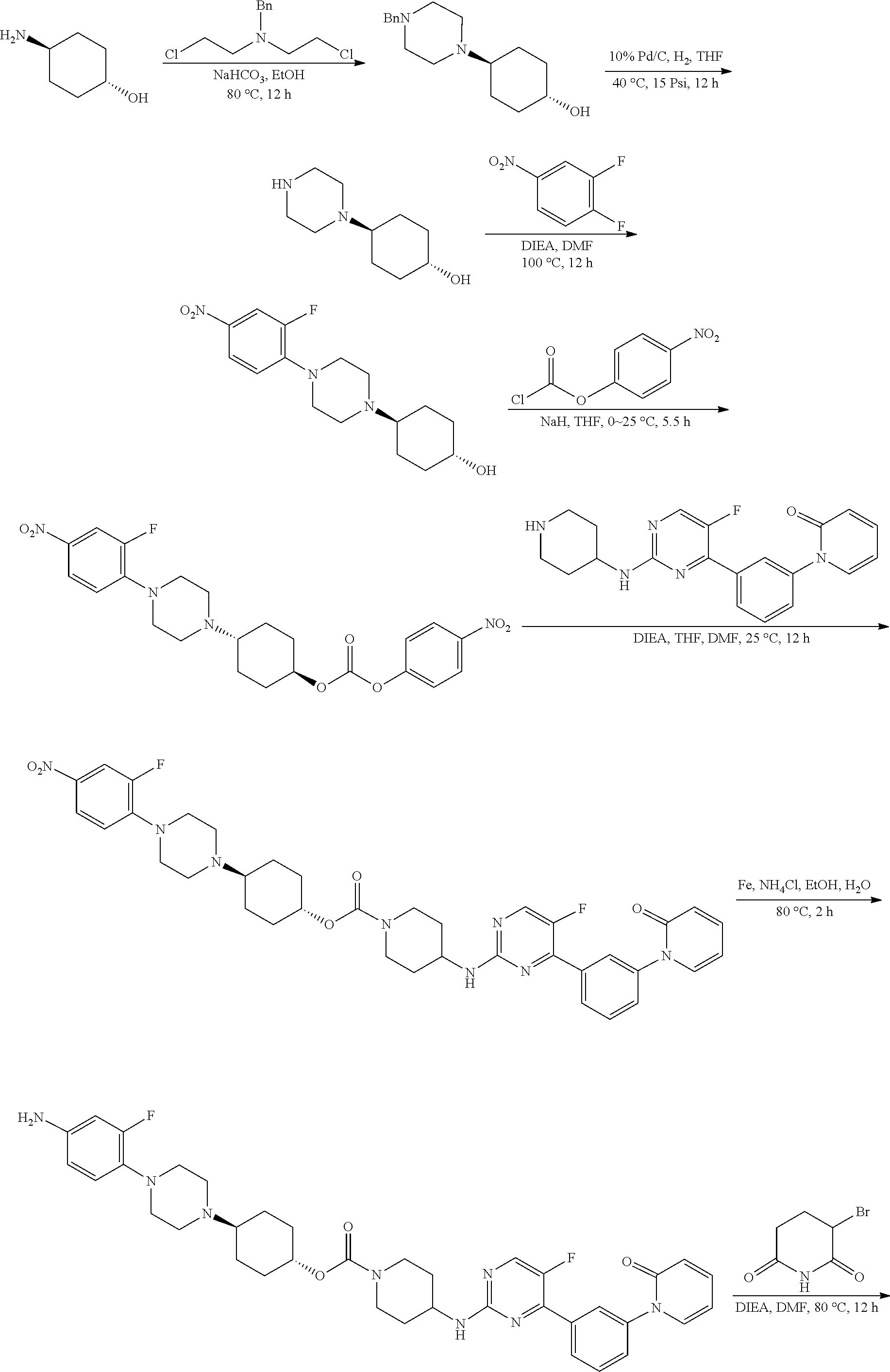

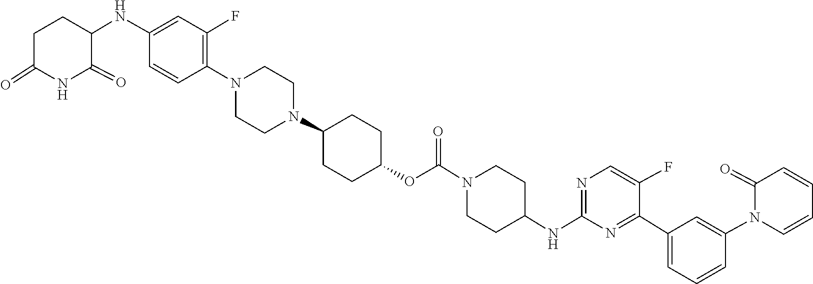 [SMILES: O=C1CCC(Nc2ccc(N3CCN([C@H]4CC[C@H](OC(=O)N5CCC(Nc6ncc(F)c(-c7cccc(-n8ccccc8=O)c7)n6)CC5)CC4)CC3)c(F)c2)C(=O)N1]

Synthesis of trans-(1r,4r)-4-(4-benzylpiperazin-1-yl)cyclohexan-1-ol. To a solution of trans-(1r,4r)-4-aminocyclohexan-1-ol (5.00 g, 43.4 mmol, 1.00 eq) and N-benzyl-2-chloro-N-(2-chloroethyl)ethan-1-amine (11.7 g, 43.4 mmol, 1.00 eq, HCl) in EtOH (100 mL) was added NaHCO₃ (10.9 g, 130 mmol, 5.07 mL, 3.00 eq) at 20 °C. The mixture was stirred at 80 °C for 12 h. LC-MS showed that trans-(1r,4r)-4-aminocyclohexan-1-ol was consumed completely and one main peak with desired MS was detected. The reaction mixture was filtered and the filtrate was concentrated under reduced pressure to remove solvent. The residue was purified by column chromatography (SiO₂, Petroleum ether/THF=100/1 to 74/26) to give the title compound (5.90 g, 21.5 mmol, 49.5% yield) as a white solid. LCMS (ES⁺): m/z 275.3 [M+H]⁺.

Synthesis of trans-(1r,4r)-4-(piperazin-1-yl)cyclohexan-1-ol. To a mixture of 10% Pd/C (1.00 g, 21.5 mmol, 50% purity, 1.00 eq) in THF (50 mL) was added trans-(1r,4r)-4-(4-benzylpiperazin-1-yl)cyclohexan-1-ol (5.90 g, 21.5 mmol, 1.00 eq) at 20 °C. The suspension was degassed and purged with H₂ for 3 times. The mixture was stirred under H₂ (15 Psi) at 40 °C for 12 h. TLC (SiO₂, Petroleum ether/THF=0/1) indicated trans-(1r,4r)-4-(4-benzylpiperazin-1-yl)cyclohexan-1-ol was consumed completely and one new spot formed. The reaction mixture was filtered and the filtrate was concentrated under reduced pressure to remove solvent to give the title compound (3.80 g, crude) as a white solid. LCMS (ES⁺): m/z 185.2 [M+H]⁺.

Synthesis of trans-(1r,4r)-4-(4-(2-fluoro-4-nitrophenyl)piperazin-1-yl)cyclohexan-1-ol. To a solution of trans-(1r,4r)-4-(piperazin-1-yl)cyclohexan-1-ol (3.80 g, 20.6 mmol, 1.00 eq) in DMF (30 mL) was added DIEA (8.00 g, 61.9 mmol, 10.8 mL, 3.00 eq) and 1,2-difluoro-4-nitrobenzene (3.28 g, 20.6 mmol, 1.00 eq) at 20 °C. The mixture was stirred at 100 °C for 12 h. LC-MS showed that trans-(1r,4r)-4-(piperazin-1-yl)cyclohexan-1-ol was consumed completely and one main peak with desired MS was detected. The reaction mixture was diluted with H₂O (25 mL) and filtered. The filter cake was washed with PE (20 mL) and dried under reduced pressure to give title compound (5.50 g, crude) as a yellow solid. LCMS (ES⁺): m/z 324.2 [M+H]⁺

Synthesis of trans-(1r,4r)-4-(4-(2-fluoro-4-nitrophenyl)piperazin-1-yl)cyclohexyl (4-nitrophenyl) carbonate. To a solution of trans-(1r,4r)-4-(4-(2-fluoro-4-nitrophenyl)piperazin-1-yl)cyclohexan-1-ol (1.00 g, 3.09 mmol, 1.00 eq) in THF (15 mL) was added NaH (371 mg, 9.28 mmol, 60% purity, 3.00 eq) at 0 °C under N₂. The mixture was stirred at 25 °C for 0.5 h. The mixture was added 4-nitrophenyl carbonochloridate (1.25 g, 6.19 mmol, 2.00 eq) at 25 °C. The mixture was stirred at 25 °C for 5 h. LC-MS showed that trans-(1r,4r)-4-(4-(2-fluoro-4-nitrophenyl)piperazin-1-yl)cyclohexan-1-ol was remained and one main peak with desired MS was detected. The reaction mixture was poured into saturated NH₄Cl solution (30 mL) and extracted with EtOAc (30 mL). The combined organic layers were dried over anhydrous Na₂SO₄, filtered and concentrated under reduced pressure. The crude product was triturated with PE/EA=5/1 (15 mL) at 25 °C for 10 min to give title compound (1.13 g, 2.31 mmol, 74.8% yield) as a yellow solid. LCMS (ES⁺): m/z 489.2 [M+H]⁺

Synthesis of trans-(1r,4r)-4-(4-(2-fluoro-4-nitrophenyl)piperazin-1-yl)cyclohexyl 4-((5-fluoro-4-(3-(2-oxopyridin-1 (2H)-yl)phenyl)pyrimidin-2-yl)amino)piperidine-1-carboxylate. To a solution of trans-(1r,4r)-4-(4-(2-fluoro-4-nitrophenyl)piperazin-1-yl)cyclohexyl (4-nitrophenyl) carbonate (699 mg, 1.74 mmol, 1.00 eq) in THF (15 mL) and DMF (5 mL) was added DIEA (450 mg, 3.48 mmol, 606 μL, 2.00 eq) and 1-(3-(5-fluoro-2-(piperidin-4-ylamino)pyrimidin-4-yl)phenyl)pyridin-2 (1H)-one (850 mg, 1.74 mmol, 1.00 eq) at 25 °C. The mixture was stirred at 25 °C for 12 h. LC-MS showed that trans-(1r,4r)-4-(4-(2-fluoro-4-nitrophenyl)piperazin-1-yl)cyclohexyl (4-nitrophenyl) carbonate was consumed completely and one main peak with desired MS was detected. The reaction mixture was diluted with H₂O (20 mL) and filtered, the filter cake was dried under reduced pressure to give a residue. The crude product was triturated with PE/EtOAc=5/1 (18 mL) at 20 °C for 5 min to give title compound (1.20 g, 1.68 mmol, 96.5% yield) as a yellow solid. LCMS (ES⁺): m/z 715.3 [M+H]⁺

Synthesis of trans-(1r,4r)-4-(4-(4-amino-2-fluorophenyl)piperazin-1-yl)cyclohexyl 4-((5-fluoro-4-(3-(2-oxopyridin-1 (2H)-yl)phenyl)pyrimidin-2-yl)amino)piperidine-1-carboxylate. To a solution of trans-(1r,4r)-4-(4-(2-fluoro-4-nitrophenyl)piperazin-1-yl)cyclohexyl 4-((5-fluoro-4-(3-(2-oxopyridin-1 (2H)-yl)phenyl)pyrimidin-2-yl)amino)piperidine-1-carboxylate (500 mg, 700 μmol, 1.00 eq) in EtOH (20 mL) and H₂O (3 mL) was added NH₄Cl (374 mg, 7.00 mmol, 10.0 eq) and Fe (391 mg, 7.00 mmol, 10.0 eq) at 20 °C. The mixture was stirred at 80 °C for 2 h. LC-MS showed that trans-(1r,4r)-4-(4-(2-fluoro-4-nitrophenyl)piperazin-1-yl)cyclohexyl 4-((5-fluoro-4-(3-(2-oxopyridin-1 (2H)-yl)phenyl)pyrimidin-2-yl)amino)piperidine-1-carboxylate was consumed completely and one main peak with desired MS was detected. The reaction mixture was filtered and the filtrate was concentrated under reduced pressure to remove solvent to give the title compound (500 mg, crude) as an off-white solid. LCMS (ES⁺): m/z 685.4 [M+H]⁺

Synthesis of trans-(1R,4r)-4-(4-(4-(((R)-2,6-dioxopiperidin-3-yl)amino)-2-fluorophenyl)piperazin-1-yl)cyclohexyl 4-((5-fluoro-4-(3-(2-oxopyridin-1 (2H)-yl)phenyl)pyrimidin-2-yl)amino)piperidine-1-carboxylate (Compound 699). To a solution of trans-(1r,4r)-4-(4-(4-amino-2-fluorophenyl)piperazin-1-yl)cyclohexyl 4-((5-fluoro-4-(3-(2-oxopyridin-1 (2H)-yl)phenyl)pyrimidin-2-yl)amino)piperidine-1-carboxylate (100 mg, 146 mol, 1.00 eq) and 3-bromopiperidine-2,6-dione (112 mg, 584 μmol, 4.00 eq) in DMF (2 mL) was added DIEA (56.6 mg, 438 μmol, 76.3 μL, 3.00 eq) at 20 °C. The mixture was stirred at 80 °C for 12 h. LC-MS showed that trans-(1r,4r)-4-(4-(4-amino-2-fluorophenyl)piperazin-1-yl)cyclohexyl 4-((5-fluoro-4-(3-(2-oxopyridin-1 (2H)-yl)phenyl)pyrimidin-2-yl)amino)piperidine-1-carboxylate was consumed completely and one main peak with desired MS was detected. The reaction mixture was diluted with ACN (15 mL), filtered and concentrated under reduced pressure. The residue was purified by prep-HPLC (column: Waters Xbridge Prep OBD C18 150*40 mm*10 um; mobile phase: [H₂O (10 mM NH₄HCO₃)-ACN]; gradient: 20%-50% B over 8.0 min). Then the residue was twice purified by prep-HPLC (column: Phenomenex luna C18 100*40 mm*3 um; mobile phase: [H₂O (0.2% FA)-ACN]; gradient: 15%-40% B over 8.0 min) to give the title compound (Compound 699) (10.2 mg, 11.9 μmol, 2.72% yield, 94.5% purity, 0.3 FA) as a pale yellow solid. LCMS (ES⁺): m/z 796.3 [M+H]⁺

### Example 619. Synthesis of trans-3-((3-fluoro-4-(1-((4-(4-((5-fluoro-4-(3-(2-oxopyridin-1 (2H)-yl)phenyl)pyrimidin-2-yl)amino)piperidine-1-carbonyl)-1-hydroxycyclohexyl)methyl)piperidin-4-yl)phenyl)amino)piperidine-2,6-dione (Compound 680)

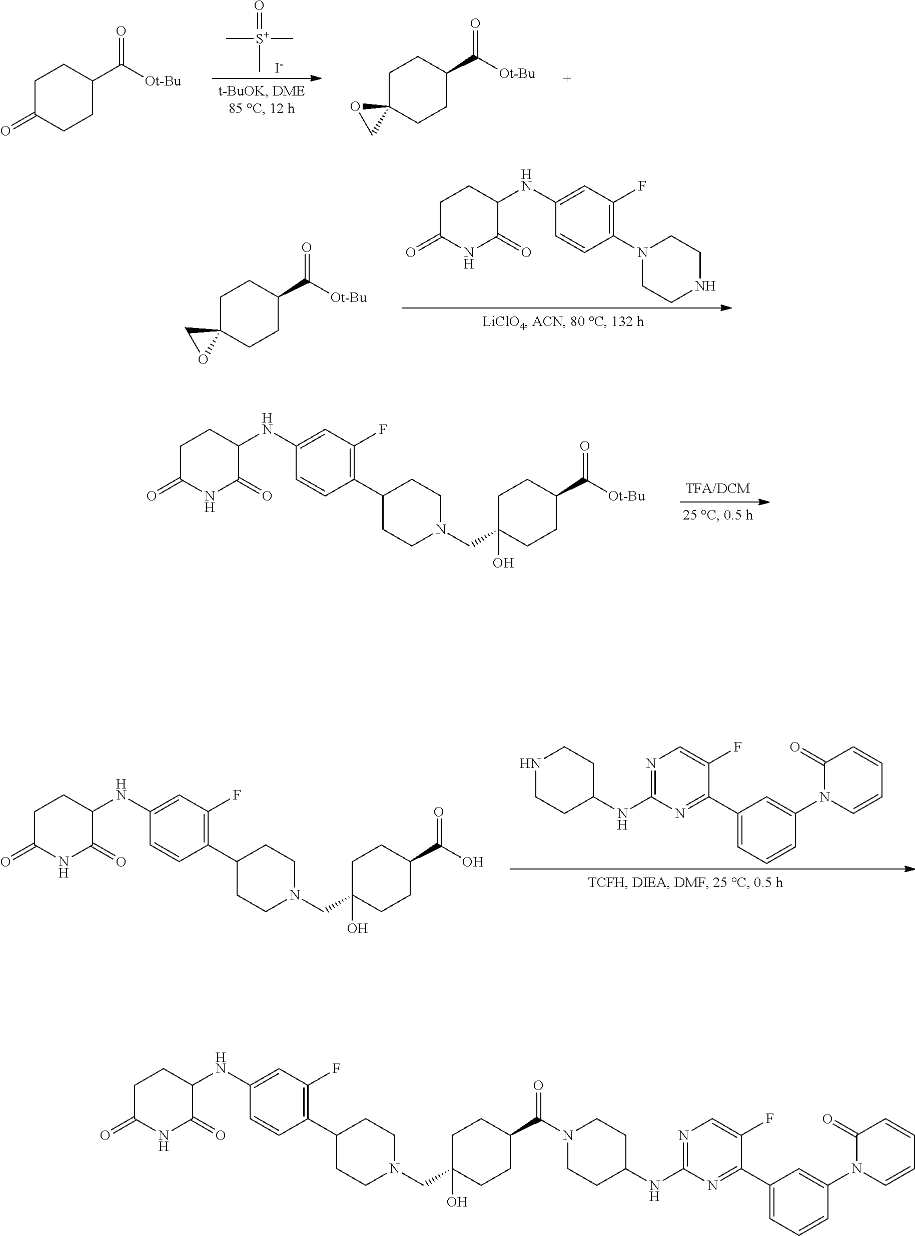

Synthesis of the mixture of tert-butyl (3s,6s)-1-oxaspiro[2.5]octane-6-carboxylate and tert-butyl (3r,6r)-1-oxaspiro[2.5]octane-6-carboxylate. To a solution of tert-butyl 4-oxocyclohexane-1-carboxylate (25.0 g, 126 mmol, 1.00 eq) in DME (800 mL) were added trimethylsulfoxide (27.8 g, 126 mmol, 1.00 eq), t-BuOK (28.3 g, 252 mmol, 2.00 eq) at 25 °C under N₂ atmosphere. The mixture was stirred at 85 °C for 12 h. TLC (SiO₂, Petroleum ether:Ethyl acetate=5:1) indicated tert-butyl 4-oxocyclohexane-1-carboxylate was consumed completely and one new spot formed. Water (300 mL) was added to the mixture at 25 °C. Then the reaction mixture was extracted with EtOAc (300 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=10:1 to 5:1) to give the title compound the mixture of and (6.00 g, 28.2 mmol, 22.4% yield) as a colorless oil. The product couldn't be detected by LCMS. It was confirmed by ¹H NMR (400 MHz, DMSO-d₆) δ=2.57 (s, 2H), 2.27-2.22 (m, 1H), 1.88-1.83 (m, 2H), 1.78-1.72 (m, 4H), 1.67 (s, 2H), 1.38 (s, 9H).

Synthesis of trans-tert-butyl 4-((4-(4-((2,6-dioxopiperidin-3-yl)amino)-2-fluorophenyl)piperidin-1-yl)methyl)-4-hydroxycyclohexane-1-carboxylate. To a solution of the mixture of tert-butyl (3s,6s)-1-oxaspiro[2.5]octane-6-carboxylate and tert-butyl (3r,6r)-1-oxaspiro[2.5]octane-6-carboxylate (3.00 g, 14.1 mmol, 1.00 eq) in ACN (400 mL) was added 3-((3-fluoro-4-(piperidin-4-yl)phenyl)amino)piperidine-2,6-dione (1.51 g, 4.95 mmol, 0.350 eq) at 25 °C. Then LiClO₄ (37.6 g, 354 mmol, 15.6 mL, 25.0 eq) was added to the mixture at 25 °C. The mixture was stirred at 80 °C for 132 h. LC-MS showed 3-[3-fluoro-4-(4-piperidyl)anilino]piperidine-2,6-dione remained and two peaks with the desired mass was detected. Saturated Na₂SO₃ aqueous solution (500 mL) was added dropwise to the mixture at 25 °C. The reaction mixture was extracted with EtOAC (300 mL*3). The organic layers were dried over anhydrous sodium sulfate, filtered and the filtrate was concentrated under reduced to give a residue. The residue was purified by prep-HPLC (column: Waters Xbridge Prep OBD C18 150*40 mm*Oum; mobile phase: [H₂O (10 mM NH₄HCO₃)-ACN]; gradient: 45%-75% B over 8.0 min) to separate the two peaks to give the title compound (100 mg, 193 μmol, 1.37% yield) as a white solid. LCMS (ES⁺): m/z 518.3 [M+H]⁺

Synthesis of trans-4-((4-(4-((2,6-dioxopiperidin-3-yl)amino)-2-fluorophenyl)piperidin-1-yl)methyl)-4-hydroxycyclohexane-1-carboxylic acid. To a solution of trans-tert-butyl 4-((4-(4-((2,6-dioxopiperidin-3-yl)amino)-2-fluorophenyl)piperidin-1-yl)methyl)-4-hydroxycyclohexane-1-carboxylate (100 mg, 193 μmol, 1.00 eq) in DCM (1.5 mL) was added TFA (0.5 mL) at 25 °C. The mixture was stirred at 25 °C for 0.5 h. LC-MS showed trans-tert-butyl 4-((4-(4-((2,6-dioxopiperidin-3-yl)amino)-2-fluorophenyl)piperidin-1-yl)methyl)-4-hydroxycyclohexane-1-carboxylate was consumed completely and the desired mass was detected. The reaction mixture was concentrated under reduced pressure to give the title compound (89.0 mg, crude) as a yellow oil. LCMS (ES⁺): m/z 462.3 [M+H]⁺

Synthesis of trans-3-((3-fluoro-4-(1-((4-(4-((5-fluoro-4-(3-(2-oxopyridin-1 (2H)-yl)phenyl)pyrimidin-2-yl)amino)piperidine-1-carbonyl)-1-hydroxycyclohexyl)methyl)piperidin-4-yl)phenyl)amino)piperidine-2,6-dione (Compound 680). To a solution of trans-4-((4-(4-((2,6-dioxopiperidin-3-yl)amino)-2-fluorophenyl)piperidin-1-yl)methyl)-4-hydroxycyclohexane-1-carboxylic acid (85.0 mg, 184 mol, 1.00 eq) in DMF (5 mL) were added TCFH (181 mg, 645 μmol, 3.50 eq), DIEA (119 mg, 922 μmol, 160 μL, 5.00 eq) and 1-(3-(5-fluoro-2-(piperidin-4-ylamino)pyrimidin-4-yl)phenyl)pyridin-2 (1H)-one (44.2 mg, 92.1 μmol, 0.500 eq, TFA) at 25 °C. The mixture was stirred at 25 °C for 0.5 h. LC-MS showed the desired mass was detected. The reaction mixture was concentrated under reduced pressure to give a residue. The residue was purified by prep-HPLC (column: Phenomenex luna C18 100*40 mm*5 um; mobile phase: [H₂O (0.04% HCl)-ACN]; gradient: 15%-35% B over 8.0 min) to give the title compound (Compound 680) (13.4 mg, 16.4 μmol, 8.91% yield, 99.0% purity) as a yellow solid. LCMS (ES⁺): m/z 809.4 [M+H]⁺

### Example 620. Synthesis of trans-3-((3-fluoro-4-(4-(4-(2-(4-((5-fluoro-4-(3-(2-oxopyridin-1 (2H)-yl)phenyl)pyrimidin-2-yl)amino)piperidin-1-yl)-2-oxoethyl)cyclohexyl)piperazin-1-yl)phenyl)amino)piperidine-2,6-dione (Compound 650)

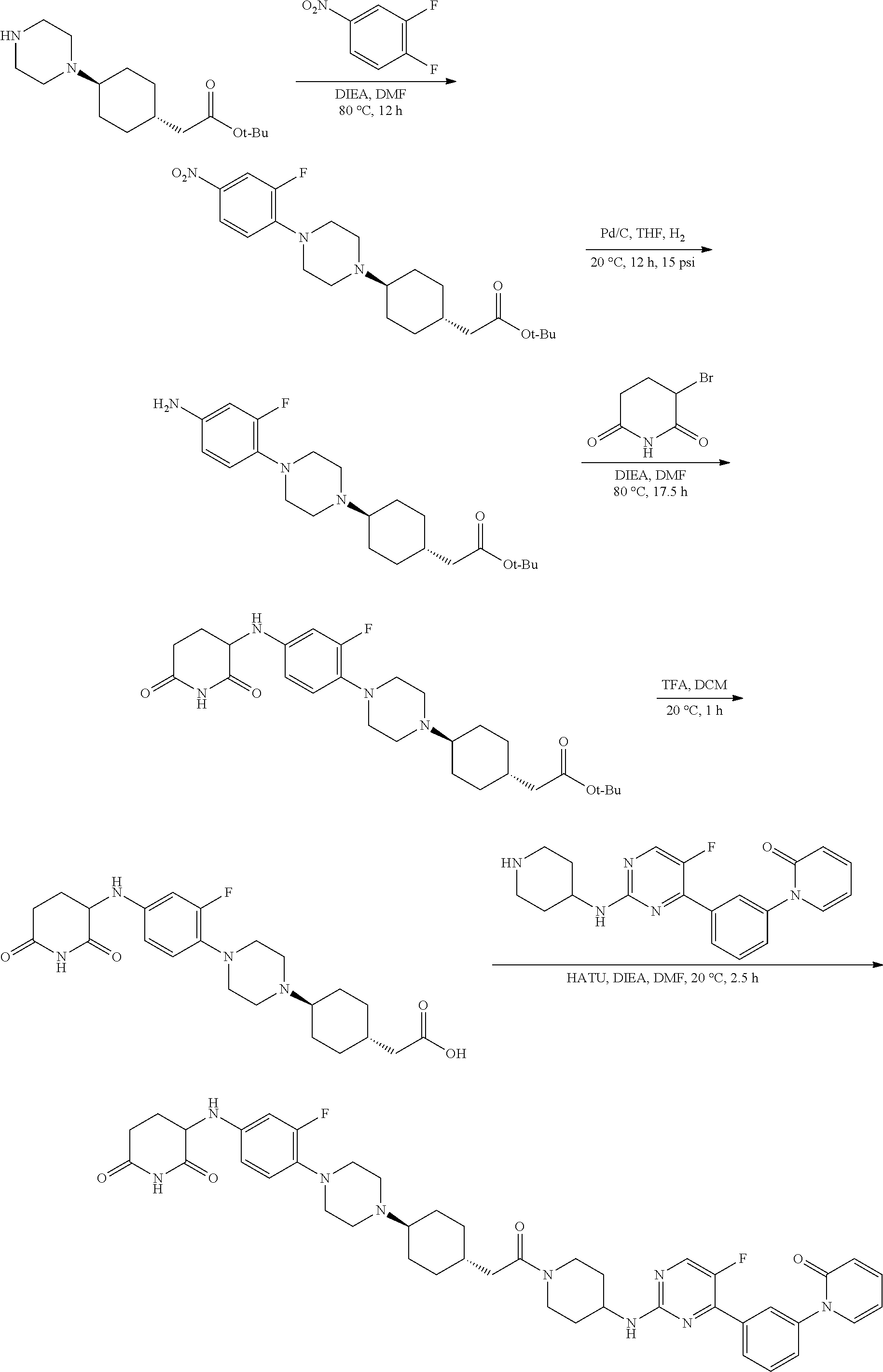

Synthesis of trans-tert-butyl 2-(4-(4-(2-fluoro-4-nitrophenyl)piperazin-1-yl)cyclohexyl)acetate. To a solution of trans-tert-butyl 2-(4-(piperazin-1-yl)cyclohexyl)acetate (200 mg, 708 μmol, 1.00 eq) in DMF (6 mL) were added DIEA (183 mg, 1.42 mmol, 247 μL, 2.00 eq) and 1,2-difluoro-4-nitrobenzene (113 mg, 708 μmol, 78.4 L, 1.00 eq) at 20 °C. The mixture was stirred at 80 °C for 12 h. LCMS showed trans-tert-butyl 2-(4-(piperazin-1-yl)cyclohexyl)acetate was consumed and the desired mass was detected. To the mixture was added H₂O (10 mL) and the mixture was extracted with EtOAc (10 mL*3). The combined organic layers were washed with brine (10 mL*3), dried over Na₂SO₄, filtered and the filtrate was concentrated under reduced pressure to give a residue. The residue was purified by prep-TLC (SiO₂, PE/EtOAc=2/1) to give the title compound (200 mg, 474 μmol, 67.0% yield) as a yellow solid. LCMS (ES+): m/z 422.3 [M+H]⁺.

Synthesis of trans-tert-butyl 2-(4-(4-(4-amino-2-fluorophenyl)piperazin-1-yl)cyclohexyl)acetate. To 10% Pd/C (200 mg, 50% purity) was added THE (2 mL) at 20 °C under N₂. Then trans-tert-butyl 2-(4-(4-(2-fluoro-4-nitrophenyl)piperazin-1-yl)cyclohexyl)acetate (200 mg, 474 μmol, 1.00 eq) was added to the mixture at 20 °C and the mixture was degassed and purged with H₂ for three times. The resulting mixture was stirred at 20 °C for 12 h H₂ (15 psi). LCMS showed trans-tert-butyl 2-(4-(4-(2-fluoro-4-nitrophenyl)piperazin-1-yl)cyclohexyl)acetate was consumed and the desired mass was detected. The mixture was filtered and the filtrate was concentrated under reduced pressure to give the title compound (186 mg, crude) as a yellow oil. LCMS (ES+): m/z 392.1 [M+H]⁺.

Synthesis of trans-tert-butyl 2-(4-(4-(4-((2,6-dioxopiperidin-3-yl)amino)-2-fluorophenyl)piperazin-1-yl)cyclohexyl)acetate. To a solution of trans-tert-butyl 2-(4-(4-(4-amino-2-fluorophenyl)piperazin-1-yl)cyclohexyl)acetate (186 mg, 475 μmol, 1.00 eq) in DMF (5 mL) were added DIEA (123 mg, 950 μmol, 166 μL, 2.00 eq) and 3-bromopiperidine-2,6-dione (274 mg, 1.43 mmol, 3.00 eq) at 20 °C. The mixture was stirred at 80 °C for 12 h. LCMS showed trans-tert-butyl 2-(4-(4-(4-amino-2-fluorophenyl)piperazin-1-yl)cyclohexyl)acetate remained and the desired mass was detected. Then 3-bromopiperidine-2,6-dione (274 mg, 1.43 mmol, 3.00 eq) was added to the mixture at 20 °C. The mixture was stirred at 80 °C for 5.5 h. LCMS showed trans-tert-butyl 2-(4-(4-(4-amino-2-fluorophenyl)piperazin-1-yl)cyclohexyl)acetate was consumed completely. To the mixture was added H₂O (10 mL) and the mixture was extracted with EtOAc (10 mL*3). The combined organic layers were washed with brine (10 mL*3), dried over Na₂SO₄, filtered and the filtrate was concentrated under reduced pressure to give a residue. The residue was purified by prep-TLC (SiO₂, PE/THF=1/1) to give the title compound (110 mg, 219 μmol, 46.1% yield) as a yellow oil. LCMS (ES+): m/z 503.3 [M+H]⁺.

Synthesis of trans-2-(4-(4-(4-((2,6-dioxopiperidin-3-yl)amino)-2-fluorophenyl)piperazin-1-yl)cyclohexyl)acetic acid. A solution of trans-tert-butyl 2-(4-(4-(4-((2,6-dioxopiperidin-3-yl)amino)-2-fluorophenyl)piperazin-1-yl)cyclohexyl)acetate (55.0 mg, 109 μmol, 1.00 eq) in DCM (1.8 mL) and TFA (0.6 mL) was stirred at 20 °C for 1 h. LCMS showed trans-tert-butyl 2-(4-(4-(4-((2,6-dioxopiperidin-3-yl)amino)-2-fluorophenyl)piperazin-1-yl)cyclohexyl)acetate was consumed and the desired mass was detected. The mixture was concentrated under reduced pressure to give the title compound (49.0 mg, crude) as a yellow oil. LCMS (ES+): m/z 447.1 [M+H]⁺.

Synthesis of trans-3-((3-fluoro-4-(4-(4-(2-(4-((5-fluoro-4-(3-(2-oxopyridin-1 (2H)-yl)phenyl)pyrimidin-2-yl)amino)piperidin-1-yl)-2-oxoethyl)cyclohexyl)piperazin-1-yl)phenyl)amino)piperidine-2,6-dione (Compound 650). To a solution of trans-2-(4-(4-(4-((2,6-dioxopiperidin-3-yl)amino)-2-fluorophenyl)piperazin-1-yl)cyclohexyl)acetic acid (49.0 mg, 110 μmol, 1.00 eq) in DMF (3 mL) were added DIEA (71.0 mg, 548 μmol, 95.5 μL, 5.00 eq) and HATU (25.0 mg, 65.8 μmol, 0.600 eq) at 20 °C. Then to the mixture was added 1-(3-(5-fluoro-2-(piperidin-4-ylamino)pyrimidin-4-yl)phenyl)pyridin-2 (1H)-one (31.6 mg, 65.8 mol, 0.600 eq, TFA) at 20 °C. The mixture was stirred at 20 °C for 2.5 h. LCMS showed 1-(3-(5-fluoro-2-(piperidin-4-ylamino)pyrimidin-4-yl)phenyl)pyridin-2 (1H)-one was consumed and the desired mass was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 26%-56% B over 8.0 min) to give the title compound (Compound 650) (4.00 mg, 4.77 μmol, 4.35% yield, 94.7% purity) as a pale yellow solid. LCMS (ES⁺): m/z 794.4 [M+H]⁺

### Example 621. Synthesis of trans-(4-((4-(4-((2,6-dioxopiperidin-3-yl)amino)-2-fluorophenyl)piperidin-1-yl)methyl)cyclohexyl)methyl 4-((5-fluoro-4-(3-(2-oxopyridin-1 (2H)-yl)phenyl)pyrimidin-2-yl)amino)piperidine-1-carboxylate (Compound 644)

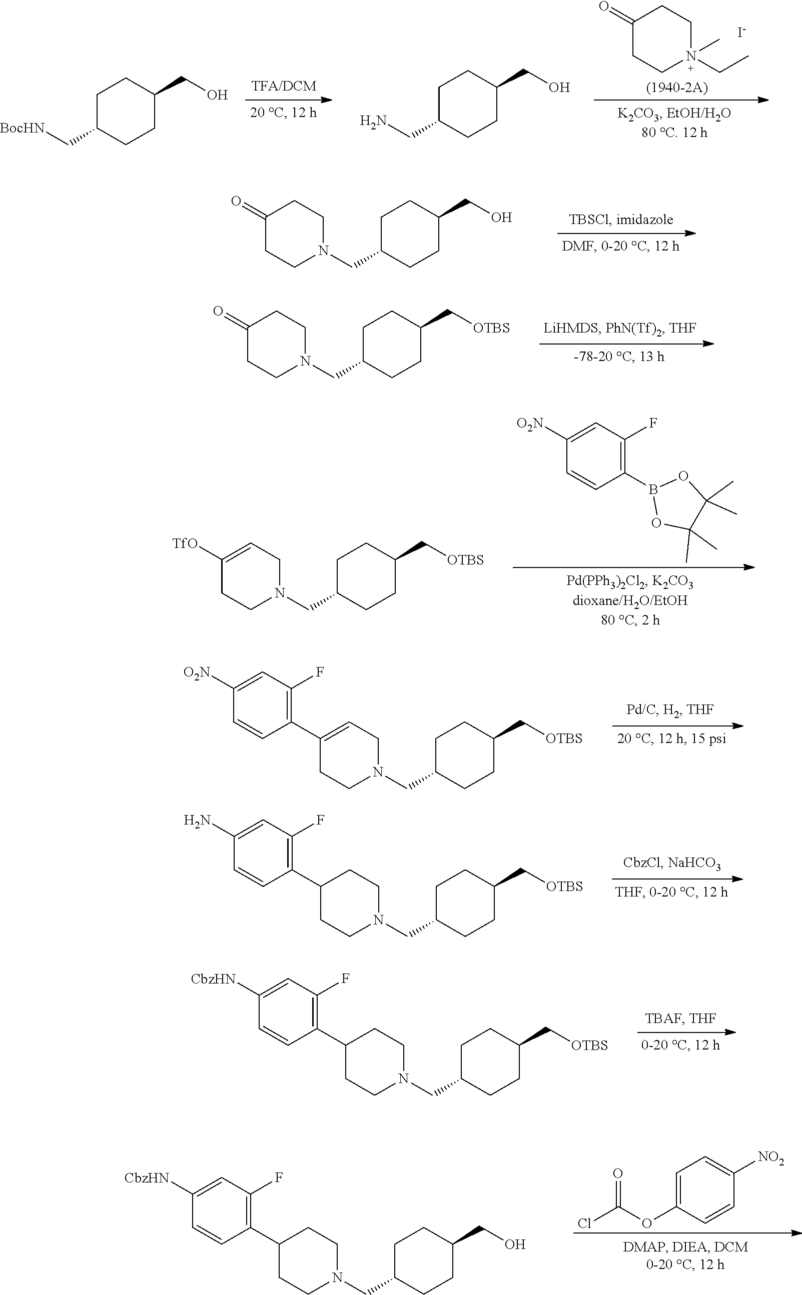

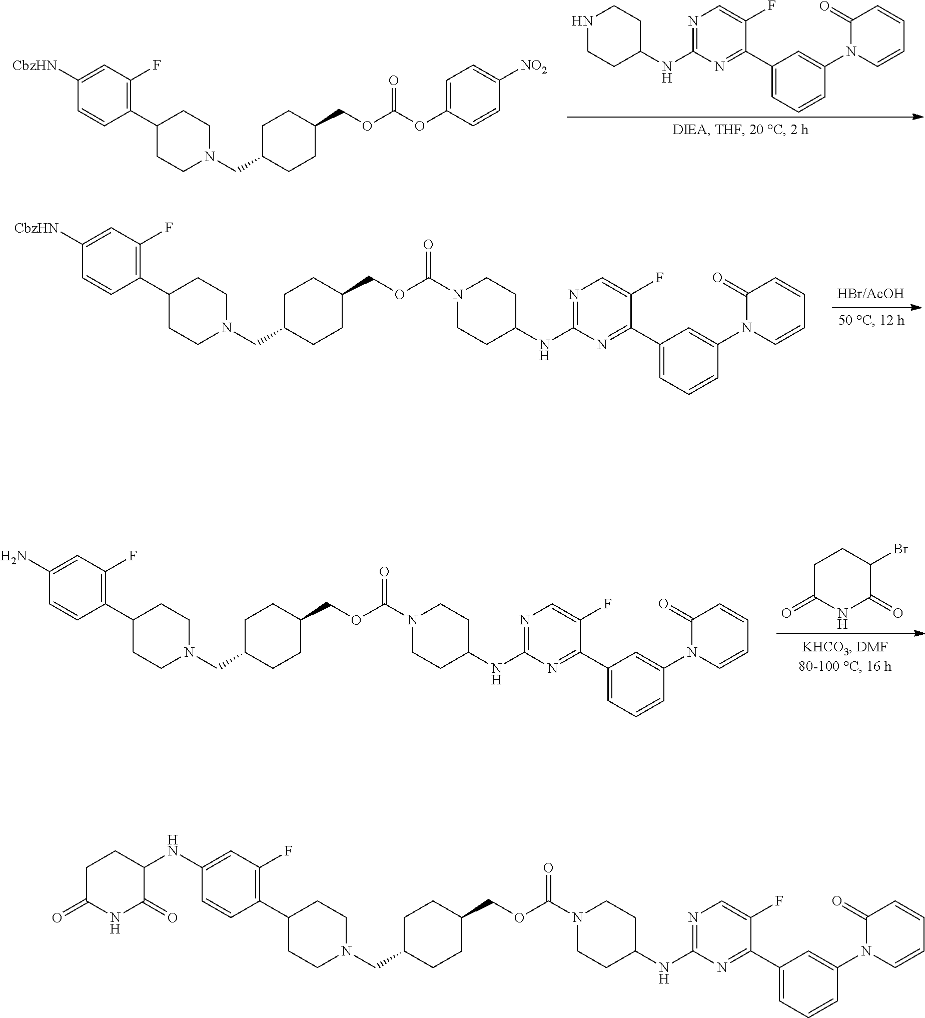

Synthesis of trans-(4-(aminomethyl)cyclohexyl)methanol. A solution of trans-tert-butyl ((4-(hydroxymethyl)cyclohexyl)methyl)carbamate (20.0 g, 82.2 mmol, 1.00 eq) in DCM (150 mL) and TFA (30 mL) was stirred at 20 °C for 12 h. LC-MS showed trans-tert-butyl ((4-(hydroxymethyl)cyclohexyl)methyl)carbamate was consumed completely and the desired mass was detected. The mixture was concentrated under reduced pressure to give the title compound (21.0 g, crude, TFA) as a colorless oil. LCMS (ES⁺): m/z 144.4 [M+H]⁺

Synthesis of trans-1-((4-(hydroxymethyl)cyclohexyl)methyl)piperidin-4-one. To a solution of trans-(4-(aminomethyl)cyclohexyl)methanol (21.0 g, 81.6 mmol, 1.00 eq, TFA) and 1-ethyl-1-methyl-4-oxopiperidin-1-ium (24.2 g, 89.8 mmol, 1.10 eq) in EtOH (400 mL) and H₂O (240 mL) was added K₂CO₃ (33.9 g, 245 mmol, 3.00 eq) at 20 °C. The mixture was stirred at 80 °C for 12 h. LCMS showed trans-(4-(aminomethyl)cyclohexyl)methanol was consumed completely and the desired mass was detected. The mixture was concentrated under reduced pressure to remove EtOH. The remained aqueous phase was extracted with EtOAc (240 mL*3). The combined organic layers were washed with brine (300 mL), dried over Na₂SO₄, filtered and the filtrate was concentrated under reduced pressure to give a residue. The residue was purified by MPLC (SiO₂, PE/EtOAc=1/0 to 0/1) to give the title compound (14.0 g, 62.1 mmol, 76.1% yield) as a yellow oil. LCMS (ES⁺): m/z 226.3 [M+H]⁺

Synthesis of trans-1-((4-(((tert-butyldimethylsilyl)oxy)methyl)cyclohexyl)methyl)piperidin-4-one. To a solution of trans-1-((4-(hydroxymethyl)cyclohexyl)methyl)piperidin-4-one (8.00 g, 35.5 mmol, 1.00 eq) in DMF (200 mL) were added IMIDAZOLE (12.1 g, 178 mmol, 5.00 eq) and tert-butyl-chloro-dimethyl-silane (10.7 g, 71.0 mmol, 8.74 mL, 2.00 eq) at 0 °C. The mixture was stirred at 20 °C for 12 h. LCMS showed trans-1-((4-(hydroxymethyl)cyclohexyl)methyl)piperidin-4-one was consumed completely and the desired mass was detected. H₂O (200 mL) was added to the mixture and the mixture was extracted with EtOAc (200 mL*3). The combined organic layers were washed with saturated aqueous NaCl solution (400 mL), dried over Na₂SO₄, filtered and the filtrate was concentrated under reduced pressure to give a residue. The residue was purified by MPLC (SiO₂, PE/EtOAc=1/0 to 0/1) to give the title compound (9.00 g, 26.5 mmol, 74.7% yield) as a colorless oil. LCMS (ES⁺): m/z 340.3 [M+H]⁺

Synthesis of trans-1-((4-(((tert-butyldimethylsilyl)oxy)methyl)cyclohexyl)methyl)-1,2,3,6-tetrahydropyridin-4-yl trifluoromethanesulfonate. To a solution of trans-1-((4-(((tert-butyldimethylsilyl)oxy)methyl)cyclohexyl)methyl)piperidin-4-one (8.00 g, 23.6 mmol, 1.00 eq) in THF (200 mL) was added LiHMDS (1.00 M, 25.9 mL, 1.10 eq) at −78 °C under N₂ atmosphere. The mixture was stirred at −78 °C for 1 h. Then a solution of 1,1,1-trifluoro-N-phenyl-N-(trifluoromethylsulfonyl)methanesulfonamide (12.6 g, 35.3 mmol, 1.50 eq) in THF (200 mL) was added to the mixture at −78 °C. The mixture was stirred at 20 °C for 12 h. LCMS showed trans-1-((4-(((tert-butyldimethylsilyl)oxy)methyl)cyclohexyl)methyl)piperidin-4-one was consumed completely and the desired mass was detected. Saturated NH₄Cl aqueous solution (400 mL) was added to the mixture to quench the reaction at 0 °C. Then the reaction mixture was extracted with EtOAc (500 mL*3). The organic layers were dried over Na₂SO₄, filtered and the filtrate was concentrated under reduced pressure to give a residue. The residue was purified by MPLC (SiO₂, PE/EtOAc=1/0 to 0/1) to give the title compound (5.50 g, 11.7 mmol, 49.5% yield) as a colorless oil. LCMS (ES⁺): m/z 472.2 [M+H]⁺

Synthesis of trans-1-((4-(((tert-butyldimethylsilyl)oxy)methyl)cyclohexyl)methyl)-4-(2-fluoro-4-nitrophenyl)-1,2,3,6-tetrahydropyridine. To a stirred solution of trans-1-((4-(((tert-butyldimethylsilyl)oxy)methyl)cyclohexyl)methyl)-1,2,3,6-tetrahydropyridin-4-yl trifluoromethanesulfonate (4.50 g, 9.54 mmol, 1.00 eq) and 2-(2-fluoro-4-nitrophenyl)-4,4,5,5-tetramethyl-1,3,2-dioxaborolane (2.80 g, 10.5 mmol, 1.10 eq) in dioxane (60 mL), H₂O (12 mL) and EtOH (24 mL) were added K₂CO₃ (3.96 g, 28.6 mmol, 3.00 eq) and Pd(PPh₃)₂Cl₂ (670 mg, 954 μmol, 0.100 eq) at 20 °C under N₂ atmosphere. The resulting mixture was stirred at 80 °C for 2 h under N₂ atmosphere. LCMS showed trans-1-((4-(((tert-butyldimethylsilyl)oxy)methyl)cyclohexyl)methyl)-1,2,3,6-tetrahydropyridin-4-yl trifluoromethanesulfonate was consumed completely and the desired mass was detected. The mixture was concentrated under reduced pressure to remove EtOH. H₂O (50 mL) was added to the mixture and the mixture was extracted with EtOAc (50 mL*3). The combined organic layers were washed with brine (50 mL*3), dried over Na₂SO₄, filtered and the filtrate was concentrated under reduced pressure to give a residue. The residue was purified by MPLC (SiO₂, PE/EtOAc=1/0 to 0/1) to give the title compound (2.40 g, 5.19 mmol, 54.4% yield) as a yellow oil. LCMS (ES⁺): m/z 463.4 [M+H]⁺

Synthesis of trans-4-(1-((4-(((tert-butyldimethylsilyl)oxy)methyl)cyclohexyl)methyl)piperidin-4-yl)-3-fluoroaniline. 10% Pd/C (600 mg, 50.0% purity) was added to the reaction flask at 20 °C under N₂ atmosphere, and THF (30 mL) was added to the reaction flask under N₂, then trans-1-((4-(((tert-butyldimethylsilyl)oxy)methyl)cyclohexyl)methyl)-4-(2-fluoro-4-nitrophenyl)-1,2,3,6-tetrahydropyridine (1.20 g, 2.59 mmol, 1.00 eq) was added to the mixture at 20 °C under N₂ and the mixture was degassed and purged with H₂ for three times. The mixture was stirred at 20 °C for 12 h under H₂ (15 psi) atmosphere. LCMS showed trans-1-((4-(((tert-butyldimethylsilyl)oxy)methyl)cyclohexyl)methyl)-4-(2-fluoro-4-nitrophenyl)-1,2,3,6-tetrahydropyridine was consumed completely and the desired mass was detected. The mixture was filtered and the filtrate was concentrated under reduced pressure to give the title compound (1.10 g, crude) as a colorless oil. LCMS (ES⁺): m/z 435.4 [M+H]⁺

Synthesis of trans-benzyl (4-(1-((4-(((tert-butyldimethylsilyl)oxy)methyl)cyclohexyl)methyl)piperidin-4-yl)-3-fluorophenyl)carbamate. To a solution of trans-4-(1-((4-(((tert-butyldimethylsilyl)oxy)methyl)cyclohexyl)methyl)piperidin-4-yl)-3-fluoroaniline (1.10 g, 2.53 mmol, 1.00 eq) in THE (20 mL) were added NaHCO₃ (425 mg, 5.06 mmol, 197 μL, 2.00 eq) and CbzCl (475 mg, 2.78 mmol, 397 μL, 1.10 eq) at 0 °C. The mixture was stirred at 20 °C for 12 h. LC-MS showed trans-4-(1-((4-(((tert-butyldimethylsilyl)oxy)methyl)cyclohexyl)methyl)piperidin-4-yl)-3-fluoroaniline was consumed mostly and the desired mass was detected. To the mixture was added H₂O (20 mL) and the mixture was extracted with EtOAc (40 mL*3). The combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (1.40 g, 2.46 mmol, 97.3% yield) as a yellow solid. LCMS (ES⁺): m/z 569.4 [M+H]⁺

Synthesis of trans-benzyl (3-fluoro-4-(1-((4-(hydroxymethyl)cyclohexyl)methyl)piperidin-4-yl)phenyl)carbamate. To a stirred solution of trans-benzyl (4-(1-((4-(((tert-butyldimethylsilyl)oxy)methyl)cyclohexyl)methyl)piperidin-4-yl)-3-fluorophenyl)carbamate (1.40 g, 2.46 mmol, 1.00 eq) in THE (20 mL) was added TBAF (1.00 M, 4.92 mL, 2.00 eq) at 0 °C. The resulting mixture was stirred at 20 °C for 12 h. LCMS showed trans-benzyl (4-(1-((4-(((tert-butyldimethylsilyl)oxy)methyl)cyclohexyl)methyl)piperidin-4-yl)-3-fluorophenyl)carbamate was consumed completely and the desired mass was detected. H₂O (20 mL) was added to the residue and the mixture was extracted with EtOAc (20 mL*3). The combined organic layers were dried over Na₂SO₄, filtered and the filtrate was concentrated under reduced pressure to give a residue. The residue was purified by MPLC (SiO₂, PE/EtOAc=1/0 to 0/1) to give the title compound (600 mg, 1.32 mmol, 53.6% yield) as a yellow solid. LCMS (ES⁺): m/z 455.3 [M+H]⁺

Synthesis of trans-benzyl (3-fluoro-4-(1-((4-((((4-nitrophenoxy)carbonyl)oxy)methyl)cyclohexyl)methyl)piperidin-4-yl)phenyl)carbamate. To a solution of trans-benzyl (3-fluoro-4-(1-((4-(hydroxymethyl)cyclohexyl)methyl)piperidin-4-yl)phenyl)carbamate (600 mg, 1.32 mmol, 1.00 eq) and 4-nitrophenyl carbonochloridate (213 mg, 1.06 mmol, 0.800 eq) in DCM (10 mL) were added DIEA (512 mg, 3.96 mmol, 690 μL, 3.00 eq) and DMAP (129 mg, 1.06 mmol, 0.800 eq) at 0 °C, the mixture was slowly warmed to 20 °C and stirred at 20 °C for 12 h. LCMS showed trans-benzyl (3-fluoro-4-(1-((4-(hydroxymethyl)cyclohexyl)methyl)piperidin-4-yl)phenyl)carbamate was consumed completely and desired mass was detected. To the mixture was added H₂O (10 mL) and the mixture was extracted with DCM (10 mL*3). The combined organic layers were dried over Na₂SO₄, filtered and the filtrate was concentrated under reduced pressure to give a residue. The residue was purified by prep-TLC (SiO₂, PE/EtOAc=2/1) to give the title compound (130 mg, 210 μmol, 15.9% yield) as a yellow oil. LCMS (ES⁺): m/z 620.3 [M+H]⁺

Synthesis of trans-(4-((4-(4-(((benzyloxy)carbonyl)amino)-2-fluorophenyl)piperidin-1-yl)methyl)cyclohexyl)methyl 4-((5-fluoro-4-(3-(2-oxopyridin-1 (2H)-yl)phenyl)pyrimidin-2-yl)amino)piperidine-1-carboxylate. To a solution of trans-benzyl (3-fluoro-4-(1-((4-((((4-nitrophenoxy)carbonyl)oxy)methyl)cyclohexyl)methyl)piperidin-4-yl)phenyl)carbamate (128 mg, 207 μmol, 0.690 eq) and 1-(3-(5-fluoro-2-(piperidin-4-ylamino)pyrimidin-4-yl)phenyl)pyridin-2 (1H)-one (144 mg, 300 μmol, 1.00 eq, TFA) in THF (3 mL) was added DIEA (194 mg, 1.50 mmol, 263 μL, 5.00 eq) at 20 °C, then the mixture was stirred at 20 °C for 2 h. LCMS showed trans-benzyl (3-fluoro-4-(1-((4-((((4-nitrophenoxy)carbonyl)oxy)methyl)cyclohexyl)methyl)piperidin-4-yl)phenyl)carbamate was consumed completely and the desired mass was detected. To the mixture was added H₂O (3 mL) and the mixture was extracted with EtOAc (6 mL*3), the combined organic layers were dried over Na₂SO₄, filtered and the filtrate was concentrated under reduced pressure to give a residue. The residue was purified by prep-TLC (SiO₂, PE/EtOAc=0/1) to give the title compound (130 mg, 154 μmol, 51.2% yield) as a yellow oil. LCMS (ES⁺): m/z 846.5 [M+H]⁺

Synthesis of trans-(4-((4-(4-amino-2-fluorophenyl)piperidin-1-yl)methyl)cyclohexyl)methyl 4-((5-fluoro-4-(3-(2-oxopyridin-1 (2H)-yl)phenyl)pyrimidin-2-yl)amino)piperidine-1-carboxylate. A solution of trans-(4-((4-(4-(((benzyloxy)carbonyl)amino)-2-fluorophenyl)piperidin-1-yl)methyl)cyclohexyl)methyl 4-((5-fluoro-4-(3-(2-oxopyridin-1 (2H)-yl)phenyl)pyrimidin-2-yl)amino)piperidine-1-carboxylate (130 mg, 154 μmol, 1.00 eq) in HBr (1 mL) and AcOH (2 mL) was stirred at 50 °C for 12 h. LCMS showed trans-(4-((4-(4-(((benzyloxy)carbonyl)amino)-2-fluorophenyl)piperidin-1-yl)methyl)cyclohexyl)methyl 4-((5-fluoro-4-(3-(2-oxopyridin-1 (2H)-yl)phenyl)pyrimidin-2-yl)amino)piperidine-1-carboxylate was consumed completely and the desired mass was detected. The mixture was concentrated under reduced pressure to give a residue and then the residue was dissolved in MeCN (1 mL), KHCO₃ (50 mg) was added to the mixture, the mixture was stirred at 20 °C for 1 h. Then the mixture was filtered and the filtrate was concentrated under reduced pressure to give the title compound (110 mg, crude) as a yellow oil. LCMS (ES⁺): m/z 712.4 [M+H]⁺

Synthesis of trans-(4-((4-(4-((2,6-dioxopiperidin-3-yl)amino)-2-fluorophenyl)piperidin-1-yl)methyl)cyclohexyl)methyl 4-((5-fluoro-4-(3-(2-oxopyridin-1 (2H)-yl)phenyl)pyrimidin-2-yl)amino)piperidine-1-carboxylate (Compound 644). To a stirred solution of trans-(4-((4-(4-amino-2-fluorophenyl)piperidin-1-yl)methyl)cyclohexyl)methyl 4-((5-fluoro-4-(3-(2-oxopyridin-1 (2H)-yl)phenyl)pyrimidin-2-yl)amino)piperidine-1-carboxylate (110 mg, 155 μmol, 1.00 eq) and 3-bromopiperidine-2,6-dione (89.0 mg, 464 μmol, 3.00 eq) in DMF (3 mL) was added KHCO₃ (30.9 mg, 309 μmol, 2.00 eq) at 20 °C. The resulting mixture was stirred at 80 °C for 12 h. LCMS showed trans-(4-((4-(4-amino-2-fluorophenyl)piperidin-1-yl)methyl)cyclohexyl)methyl 4-((5-fluoro-4-(3-(2-oxopyridin-1 (2H)-yl)phenyl)pyrimidin-2-yl)amino)piperidine-1-carboxylate remained and the desired mass was detected. Then to the mixture were added 3-bromopiperidine-2,6-dione (59.3 mg, 309 μmol, 2.00 eq) and KHCO₃ (30.9 mg, 309 μmol, 2.00 eq) at 20 °C, the mixture was stirred at 100 °C for 4 h. LCMS showed trans-(4-((4-(4-amino-2-fluorophenyl)piperidin-1-yl)methyl)cyclohexyl)methyl 4-((5-fluoro-4-(3-(2-oxopyridin-1 (2H)-yl)phenyl)pyrimidin-2-yl)amino)piperidine-1-carboxylate was consumed mostly and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 45%-75% B over 8.0 min) to give the title compound (Compound 644) (2.80 mg, 3.20 μmol, 2.07% yield, 93.9% purity) as a pale yellow solid. LCMS (ES⁺): m/z 823.4 [M+H]⁺

### Example 622. Synthesis of trans-4-((4-(3-(2,6-dioxopiperidin-3-yl)-1-methyl-1H-indazol-6-yl)piperidin-1-yl)methyl)cyclohexyl 4-((5-fluoro-4-(3-(2-oxopyridin-1 (2H)-yl)phenyl)pyrimidin-2-yl)amino)piperidine-1-carboxylate (Compound 679)

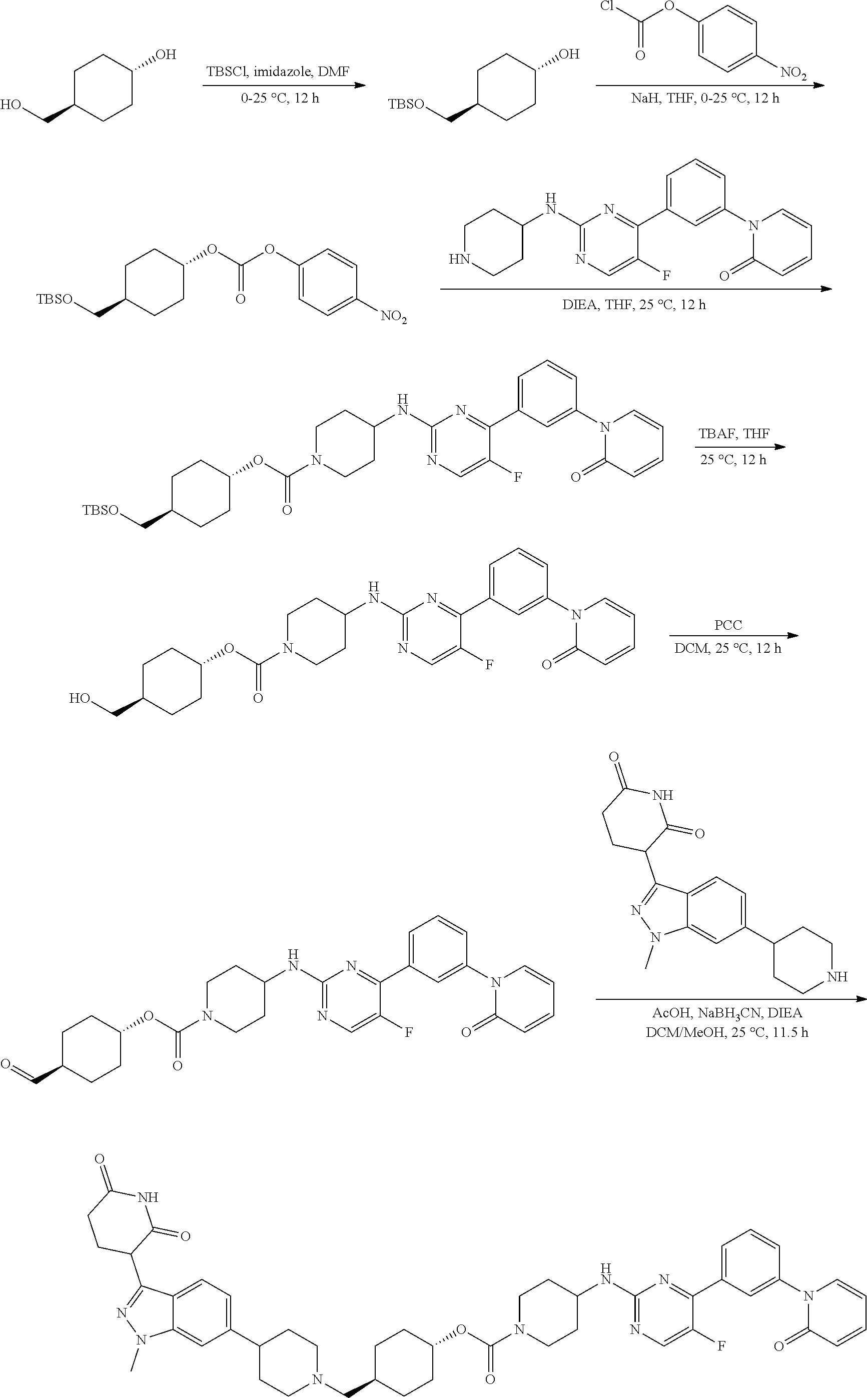

Synthesis of trans-4-(((tert-butyldimethylsilyl)oxy)methyl)cyclohexan-1-ol. To a solution of trans-4-(hydroxymethyl)cyclohexan-1-ol (3.75 g, 28.8 mmol, 1.00 eq) in DMF (200 mL) were added IMIDAZOLE (9.81 g, 144 mmol, 5.00 eq) and TBSCl (8.68 g, 57.6 mmol, 7.09 mL, 2.00 eq) at 0 °C. The mixture was stirred at 25 °C for 12 h. TLC (SiO2, Petroleum ether:Ethyl acetate=3:1) showed trans-4-(hydroxymethyl)cyclohexan-1-ol was consumed completely and three new spots formed. Water (100 mL) was added to the mixture at 25 °C. The reaction mixture was extracted with EtOAC (60 mL*5). The organic layers were dried over anhydrous sodium sulfate, filtered and the filtrate was concentrated under reduced to give a residue. The residue was purified by column chromatography (SiO₂, Petroleum ether:Ethyl acetate=5:1 to 3:1) to give the title compound (1.50, 6.14 mmol, 21.3% yield) as a light yellow oil. The product couldn't be detected by LCMS. It was confirmed by ¹H NMR (400 MHz, DMSO-d₆) δ=4.49 (d, J=4.4 Hz, 1H), 3.35 (br s, 2H), 3.30-3.24 (m, 1H), 1.83-1.77 (m, 2H), 1.70-1.63 (m, 2H), 1.35-1.21 (m, 2H), 1.14-1.03 (m, 2H), 0.94-0.89 (m, 1H), 0.85 (s, 9H), 0.02-0.01 (m, 6H).

Synthesis of trans-4-(((tert-butyldimethylsilyl)oxy)methyl)cyclohexyl (4-nitrophenyl) carbonate. To a solution of trans-4-(((tert-butyldimethylsilyl)oxy)methyl)cyclohexan-1-ol (1.39 g, 5.69 mmol, 1.00 eq) in THF (20 mL) was added NaH (1.14 g, 28.4 mmol, 60% purity, 5.00 eq) at 0 °C under N₂ atmosphere. The mixture was stirred at 0 °C for 1 h. Then 4-nitrophenyl carbonochloridate (2.86 g, 14.2 mmol, 2.50 eq) in THF (30 mL) was added to the mixture at 0 °C under N₂ atmosphere. The mixture was stirred at 25 °C for 11 h under N₂. TLC (SiO₂, Petroleum ether:Ethyl acetate=5:1) showed trans-4-(((tert-butyldimethylsilyl)oxy)methyl)cyclohexan-1-ol was consumed completely and two spots formed. To the reaction mixture was added H₂O (40 mL) at 0 °C for 20 min under N₂ atmosphere. Then the mixture extracted with EtOAc (20 mL*5). The organic layers were dried over anhydrous sodium sulfate, filtered and the filtrate was concentrated under reduced to give a residue. The residue was purified by column chromatography (SiO₂, Petroleum ether:Ethyl acetate=10:1 to 5:1) to give the title compound (1.20 g, 2.93 mmol, 51.5% yield) as a light yellow solid. The product couldn't be detected by LCMS. It was confirmed by ¹H NMR (400 MHz, DMSO-d₆) δ=8.33-8.29 (m, 2H), 7.57-7.53 (m, 2H), 4.58 (tt, J=11.1, 4.3 Hz, 1H), 3.43-3.40 (m, 2H), 2.13-2.05 (m, 2H), 1.82-1.75 (m, 2H), 1.48-1.37 (m, 3H), 1.11-1.00 (m, 2H), 0.87-0.86 (m, 9H), 0.03-0.01 (m, 6H).

Synthesis of trans-4-(((tert-butyldimethylsilyl)oxy)methyl)cyclohexyl 4-((5-fluoro-4-(3-(2-oxopyridin-1 (2H)-yl)phenyl)pyrimidin-2-yl)amino)piperidine-1-carboxylate. To a solution of trans-4-(((tert-butyldimethylsilyl)oxy)methyl)cyclohexyl (4-nitrophenyl) carbonate (1.20 g, 2.93 mmol, 1.00 eq) in THF (40 mL) were added DIEA (1.14 g, 8.79 mmol, 1.53 mL, 3.00 eq) and 1-(3-(5-fluoro-2-(piperidin-4-ylamino)pyrimidin-4-yl)phenyl)pyridin-2 (1H)-one (1.69 g, 3.52 mmol, 1.20 eq, TFA) at 25 °C. The mixture was stirred at 25 °C for 12 h. LCMS showed the desired mass was detected. The reaction mixture was concentrated under reduced pressure to give a residue. The residue was purified by column chromatography (SiO₂, Petroleum ether:Ethyl acetate=2:1 to 1:1) to give the title compound (1.70 g, 2.67 mmol, 91.3% yield) as a white solid. LCMS (ES⁺): m/z 636.4 [M+H]⁺.

Synthesis of trans-4-(hydroxymethyl)cyclohexyl 4-((5-fluoro-4-(3-(2-oxopyridin-1 (2H)-yl)phenyl)pyrimidin-2-yl)amino)piperidine-1-carboxylate. To a solution of trans-4-(((tert-butyldimethylsilyl)oxy)methyl)cyclohexyl 4-((5-fluoro-4-(3-(2-oxopyridin-1 (2H)-yl)phenyl)pyrimidin-2-yl)amino)piperidine-1-carboxylate (1.70 g, 2.67 mmol, 1.00 eq) in THF (50 mL) was added TBAF (2.80 g, 5.61 mmol, 4.00 eq) at 25 °C. The mixture was stirred at 25 °C for 12 h. LCMS showed trans-4-(((tert-butyldimethylsilyl)oxy)methyl)cyclohexyl 4-((5-fluoro-4-(3-(2-oxopyridin-1 (2H)-yl)phenyl)pyrimidin-2-yl)amino)piperidine-1-carboxylate was consumed completely and the desired mass was detected. Water (20 mL) was added to the mixture at 25 °C. The reaction mixture was extracted with EtOAc (30 mL*6). The organic layers were dried over anhydrous sodium sulfate, filtered and the filtrate was concentrated under reduced to give a residue. The residue was purified by column chromatography (SiO₂, Petroleum ether:Tetrahydrofuran=2:1 to 1:1) to give the title compound (1.39 g, crude) as a light yellow solid. LCMS (ES⁺): m/z 522.3 [M+H]⁺.

Synthesis of trans-4-formylcyclohexyl 4-((5-fluoro-4-(3-(2-oxopyridin-1 (2H)-yl)phenyl)pyrimidin-2-yl)amino)piperidine-1-carboxylate. To a solution of trans-4-(hydroxymethyl)cyclohexyl 4-((5-fluoro-4-(3-(2-oxopyridin-1 (2H)-yl)phenyl)pyrimidin-2-yl)amino)piperidine-1-carboxylate (640 mg, 1.23 mmol, 1.00 eq) in DCM (20 mL) was added PCC (793 mg, 3.68 mmol, 3.00 eq) at 25 °C. The mixture was stirred at 25 °C for 12 h. LCMS showed trans-4-(hydroxymethyl)cyclohexyl 4-((5-fluoro-4-(3-(2-oxopyridin-1 (2H)-yl)phenyl)pyrimidin-2-yl)amino)piperidine-1-carboxylate was consumed completely and the desired mass was detected. The reaction mixture was concentrated under reduced pressure to give a residue. Water (10 mL) was added to the residue at 25 °C. The aqueous phase was extracted with EtOAc (5 mL*6). The organic layers were dried over anhydrous sodium sulfate, filtered and the filtrate was concentrated under reduced to give the title compound (290 mg, crude) as a light yellow oil. LCMS (ES⁺): m/z 520.3 [M+H]⁺.

Synthesis of trans-4-((4-(3-(2,6-dioxopiperidin-3-yl)-1-methyl-1H-indazol-6-yl)piperidin-1-yl)methyl)cyclohexyl 4-((5-fluoro-4-(3-(2-oxopyridin-1 (2H)-yl)phenyl)pyrimidin-2-yl)amino)piperidine-1-carboxylate (Compound 679). To a solution of 3-[1-methyl-6-(4-piperidyl)indazol-3-yl]piperidine-2,6-dione (135 mg, 307 μmol, 1.10 eq, TFA) in DCM (3 mL) and MeOH (3 mL) was added DIEA to adjust pH to 8 at 25 °C. The mixture was stirred at 25 °C for 0.5 h. To the reaction was added were added CH₃COOH (33.52 mg, 558 μmol, 32.0 μL, 2.00 eq) and (4-formylcyclohexyl) 4-[[5-fluoro-4-[3-(2-oxo-1-pyridyl)phenyl]pyrimidin-2-yl]amino]piperidine-1-carboxylate (290 mg, 279 μmol, 1.00 eq) at 25 °C, pH was 5 now. The mixture was stirred at 25 °C for 1 hr. Then NaBH₃CN (52.6 mg, 837 μmol, 3.00 eq) was added to the mixture at 25 °C. The mixture was stirred at 25 °C for 10 hr. LCMS showed trans-4-formylcyclohexyl 4-((5-fluoro-4-(3-(2-oxopyridin-1 (2H)-yl)phenyl)pyrimidin-2-yl)amino)piperidine-1-carboxylate was consumed completely and the desired mass was detected. The reaction mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 35%-65% B over 8.0 min) to give the title compound (Compound 679) (20.9 mg, 23.0 μmol, 8.22% yield, 91.2% purity) as an off-white solid. LCMS (ES⁺): m/z 830.4 [M+H]⁺.

### Example 623. Synthesis of 2-(1-(4-((2,6-dioxopiperidin-3-yl)amino)-2-fluorophenyl)piperidin-4-yl)-2-azaspiro[3.3]heptan-6-yl 4-((5-fluoro-4-(3-(2-oxopyridin-1 (2H)-yl)phenyl)pyrimidin-2-yl)amino)piperidine-1-carboxylate (Compound 637)

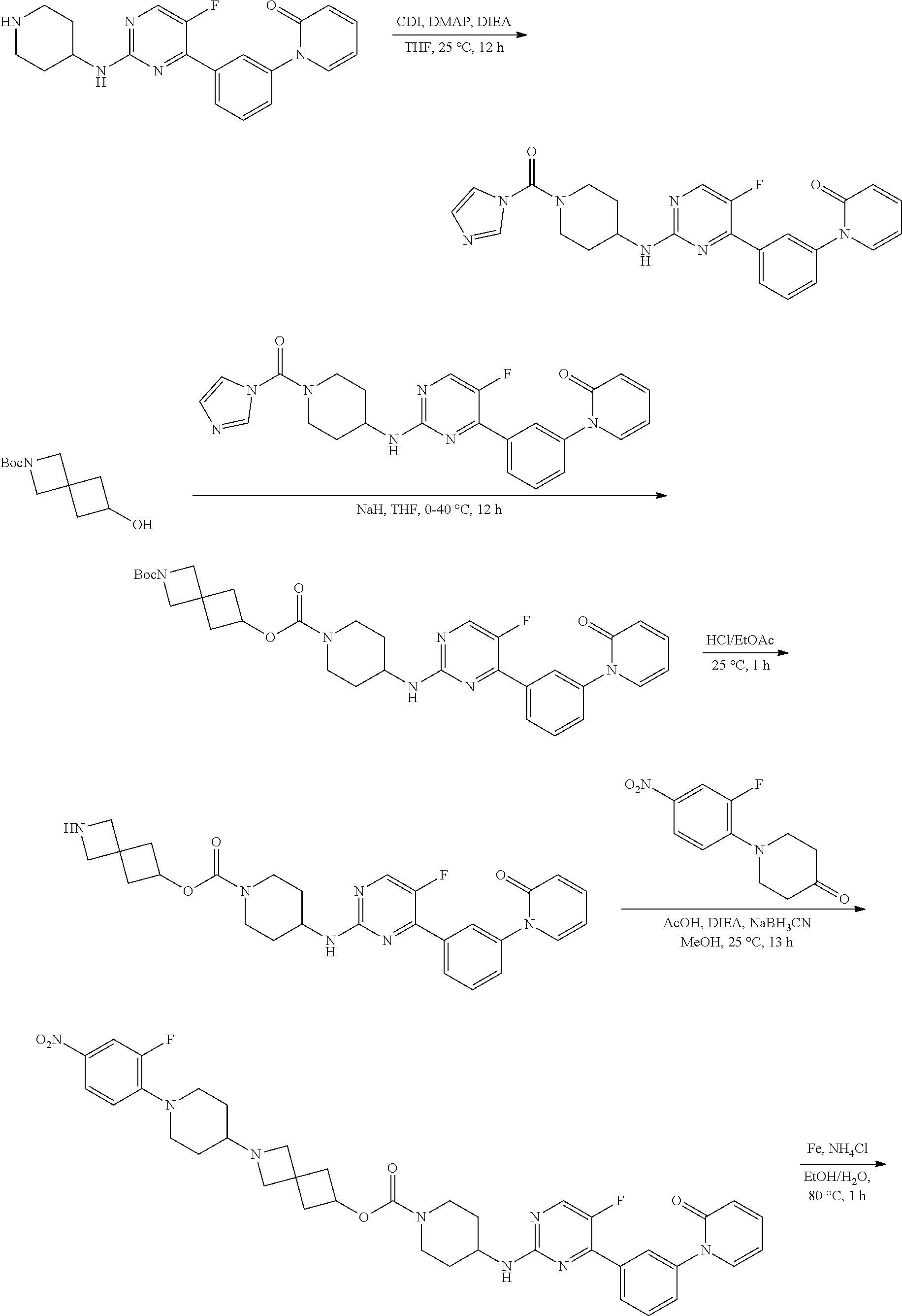

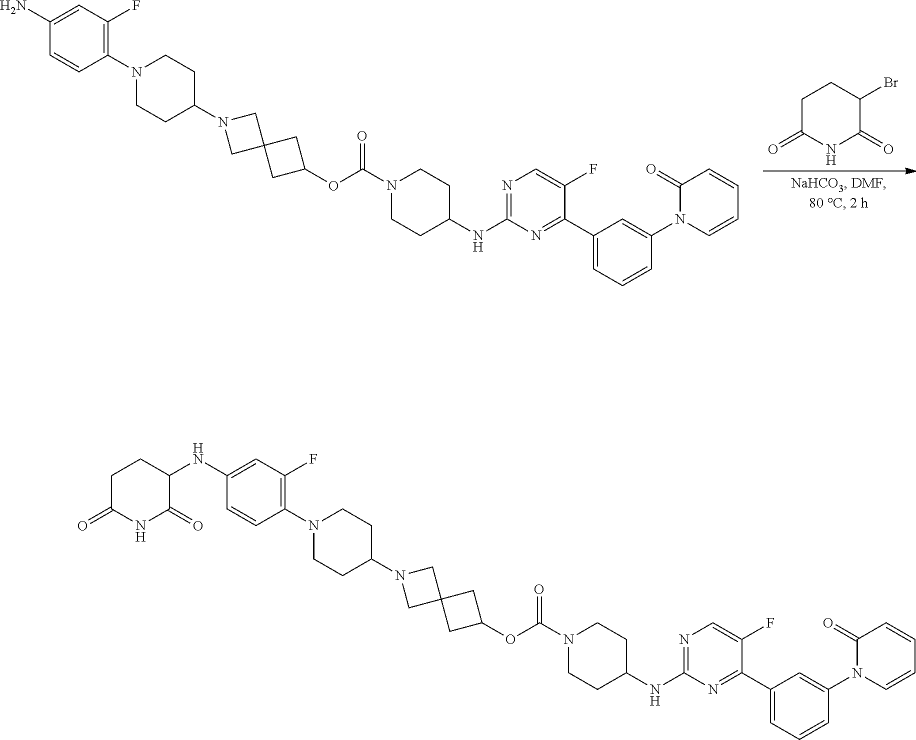 [SMILES: Nc1ccc(N2CCC(N3CC4(CC(OC(=O)N5CCC(Nc6ncc(F)c(-c7cccc(-n8ccccc8=O)c7)n6)CC5)C4)C3)CC2)c(F)c1 ; O=C1CCC(Br)C(=O)N1 ; O=C1CCC(Nc2ccc(N3CCC(N4CC5(CC(OC(=O)N6CCC(Nc7ncc(F)c(-c8cccc(-n9ccccc9=O)c8)n7)CC6)C5)C4)CC3)c(F)c2)C(=O)N1]

Synthesis of 1-[3-[5 fluoro-2-[[1-(imidazole-1-carbonyl)-4-piperidyl]amino]pyrimidin-4-yl]phenyl]pyridin-2-one. To a solution of 1-[3-[5-fluoro-2-(4-piperidylamino)pyrimidin-4-yl]phenyl]pyridin-2-one (2.00 g, 4.98 mmol, 1.00 eq, HCl) in THF (25 mL) was added DIEA (1.93 g, 14.9 mmol, 2.60 mL, 3 eq) and di(imidazol-1-yl)methanone (2.42 g, 14.9 mmol, 3.00 eq) and DMAP (608 mg, 4.98 mmol, 1.00 eq) at 25 °C. The mixture was stirred at 25 °C for 12 h. LC-MS showed 1-[3-[5-fluoro-2-(4-piperidylamino)pyrimidin-4-yl]phenyl]pyridin-2-one was consumed completely and the desired mass was detected. The reaction mixture was diluted with water 20 mL and extracted with EtOAc (25 mL*3). The combined organic layers dried over Na₂SO₄, filtered and concentrated under reduced pressure. The residue was purified by column chromatography (SiO₂, DCM/THF=1/0 to 1/1) to give the title compound (3.1 g, crude) as a brown oil. LCMS (ES⁺): m/z 460.2 [M+H]⁺.

Synthesis of tert-butyl 6-((4-((5-fluoro-4-(3-(2-oxopyridin-1 (2H)-yl)phenyl)pyrimidin-2-yl)amino)piperidine-1-carbonyl)oxy)-2-azaspiro[3.3]heptane-2-carboxylate. To a solution of tert-butyl 6-hydroxy-2-azaspiro[3.3]heptane-2-carboxylate (1.20 g, 5.63 mmol, 1.00 eq) in THF (10 mL) was added NaH (450 mg, 11.2 mmol, 60% purity, 2.00 eq) at 0 °C under N₂ atmosphere. The mixture was stirred at 40 °C for 1 h. Then 1-(3-(2-((1-(1H-imidazole-1-carbonyl)piperidin-4-yl)amino)-5-fluoropyrimidin-4-yl)phenyl)pyridin-2 (1H)-one (2.59 g, 5.63 mmol, 1.00 eq) in THF (10 mL) was added to the mixture at 40 °C. The mixture was stirred at 40 °C for 11 h. LC-MS showed the desired mass was detected. The reaction mixture was diluted with water 20 mL at 25 °C, and extracted with EtOAc (30 mL*3). The combined organic layers dried over Na₂SO₄, filtered and concentrated under reduced pressure. The residue was purified by column chromatography (SiO₂, Dichloromethane:Methanol=1:0 to 3:1) to give the title compound (1.30 g, 1.96 mmol, 34.7% yield, 91.0% purity) as a yellow oil. LCMS (ES⁺): m/z 605.3 [M+H]⁺.

Synthesis of 2-azaspiro[3.3]heptan-6-yl 4-((5-fluoro-4-(3-(2-oxopyridin-1 (2H)-yl)phenyl)pyrimidin-2-yl)amino)piperidine-1-carboxylate. To a solution of tert-butyl 6-((4-((5-fluoro-4-(3-(2-oxopyridin-1 (2H)-yl)phenyl)pyrimidin-2-yl)amino)piperidine-1-carbonyl)oxy)-2-azaspiro[3.3]heptane-2-carboxylate (1.30 g, 2.15 mmol, 1.00 eq) in HCl/EtOAc (4 M, 13.0 mL, 24.1 eq) at 25 °C. The mixture was stirred at 25 °C for 1 h. LC-MS showed the desired mass was detected. The reaction mixture was concentrated under reduced pressure to give the title compound (1.30 g, crude, HCl) as a yellow solid. LCMS (ES⁺): m/z 505.3 [M+H]⁺.

Synthesis of 2-(1-(2-fluoro-4-nitrophenyl)piperidin-4-yl)-2-azaspiro[3.3]heptan-6-yl 4-((5-fluoro-4-(3-(2-oxopyridin-1 (2H)-yl)phenyl)pyrimidin-2-yl)amino)piperidine-1-carboxylate. To a solution of 2-azaspiro[3.3]heptan-6-yl 4-((5-fluoro-4-(3-(2-oxopyridin-1 (2H)-yl)phenyl)pyrimidin-2-yl)amino)piperidine-1-carboxylate (1.30 g, 2.40 mmol, 1.00 eq, HCl) in MeOH (20 mL) was added DIEA (931 mg, 7.21 mmol, 1.26 mL, 3.00 eq) at 25 °C, The mixture was stirred at 25 °C for 0.5 h. Then AcOH (721 mg, 12.0 mmol, 687 μL, 5.00 eq) and 1-(2-fluoro-4-nitrophenyl)piperidin-4-one (2.29 g, 9.61 mmol, 4.00 eq) was added to the mixture at 25 °C, The mixture was stirred at 25 °C for 0.5 h. Then NaBH₃CN (755 mg, 12.0 mmol, 5.00 eq) to the mixture at 25 °C. The mixture was stirred at 25 °C for 12 h. LC-MS showed the desired mass was detected. The residue was poured into water (10 mL), and the reaction mixture was concentrated under reduced pressure to give a residue. The residue diluted with water 20 mL at 25 °C, and extracted with EtOAc (20 mL*3). The combined organic layers dried over Na₂SO₄, filtered and concentrated under reduced pressure. The residue was purified by column chromatography (SiO₂, Dichloromethane:Methanol=1:0 to 5:1) to give the title compound (460 mg, 625 μmol, 26.0% yield, 98.7% purity) as a yellow oil. LCMS (ES⁺): m/z 727.3 [M+H]⁺.

Synthesis of 2-(1-(4-amino-2-fluorophenyl)piperidin-4-yl)-2-azaspiro[3.3]heptan-6-yl 4-((5-fluoro-4-(3-(2-oxopyridin-1 (2H)-yl)phenyl)pyrimidin-2-yl)amino)piperidine-1-carboxylate. To a solution of 2-(1-(2-fluoro-4-nitrophenyl)piperidin-4-yl)-2-azaspiro[3.3]heptan-6-yl 4-((5-fluoro-4-(3-(2-oxopyridin-1 (2H)-yl)phenyl)pyrimidin-2-yl)amino)piperidine-1-carboxylate (340 mg, 467 μmol, 1.00 eq) in EtOH (2 mL) and H₂O (0.4 mL) was added Fe (130 mg, 2.34 mmol, 5.00 eq) and NH₄Cl (125 mg, 2.34 mmol, 5.00 eq) at 25 °C. The mixture was stirred at 80 °C for 1 h. LC-MS showed the desired mass was detected. The reaction mixture was filtered and the filtrate concentrated under high vacuum to give the title compound (350 mg, crude) as a yellow solid. LCMS (ES⁺): m/z 697.4 [M+H]⁺.

Synthesis of 2-(1-(4-((2,6-dioxopiperidin-3-yl)amino)-2-fluorophenyl)piperidin-4-yl)-2-azaspiro[3.3]heptan-6-yl 4-((5-fluoro-4-(3-(2-oxopyridin-1 (2H)-yl)phenyl)pyrimidin-2-yl)amino)piperidine-1-carboxylate (Compound 637). To a solution of 2-(1-(4-amino-2-fluorophenyl)piperidin-4-yl)-2-azaspiro[3.3]heptan-6-yl 4-((5-fluoro-4-(3-(2-oxopyridin-1 (2H)-yl)phenyl)pyrimidin-2-yl)amino)piperidine-1-carboxylate (350 mg, 502 μmol, 1.00 eq) in DMF (5 mL) was added NaHCO₃ (126 mg, 1.51 mmol, 58.6 μL, 3.00 eq) and 3-bromopiperidine-2,6-dione (289 mg, 1.51 mmol, 3.00 eq) at 25 °C. The mixture was stirred at 80 °C for 2 h. LC-MS showed the desired mass was detected. The reaction mixture was filtered to give a residue. The residue was purified by prep-HPLC (column: Phenomenex luna C18 100*40 mm*5 um; mobile phase: [H₂O (0.2% FA)-ACN]; gradient: 10%-50% B over 8.0 min). The residue was purified by prep-HPLC (column: Waters Xbridge BEH C18 100*30 mm*10 um; mobile phase: [H₂O (10 mM NH₄HCO₃)-ACN]; gradient: 35%-65% B over 8.0 min) to give the title compound (Compound 637) (10.2 mg, 12.3 μmol, 2.45% yield, 97.40% purity) as a white solid. LCMS (ES⁺): m/z 808.4 [M+H]⁺.

### Example 624. Synthesis of 6-(4-(4-((2,6-dioxopiperidin-3-yl)amino)-2-fluorophenyl)piperidin-1-yl)spiro[3.3]heptan-2-yl 4-((5-fluoro-4-(3-(2-oxopyridin-1 (2H)-yl)phenyl)pyrimidin-2-yl)amino)piperidine-1-carboxylate (Compound 633)

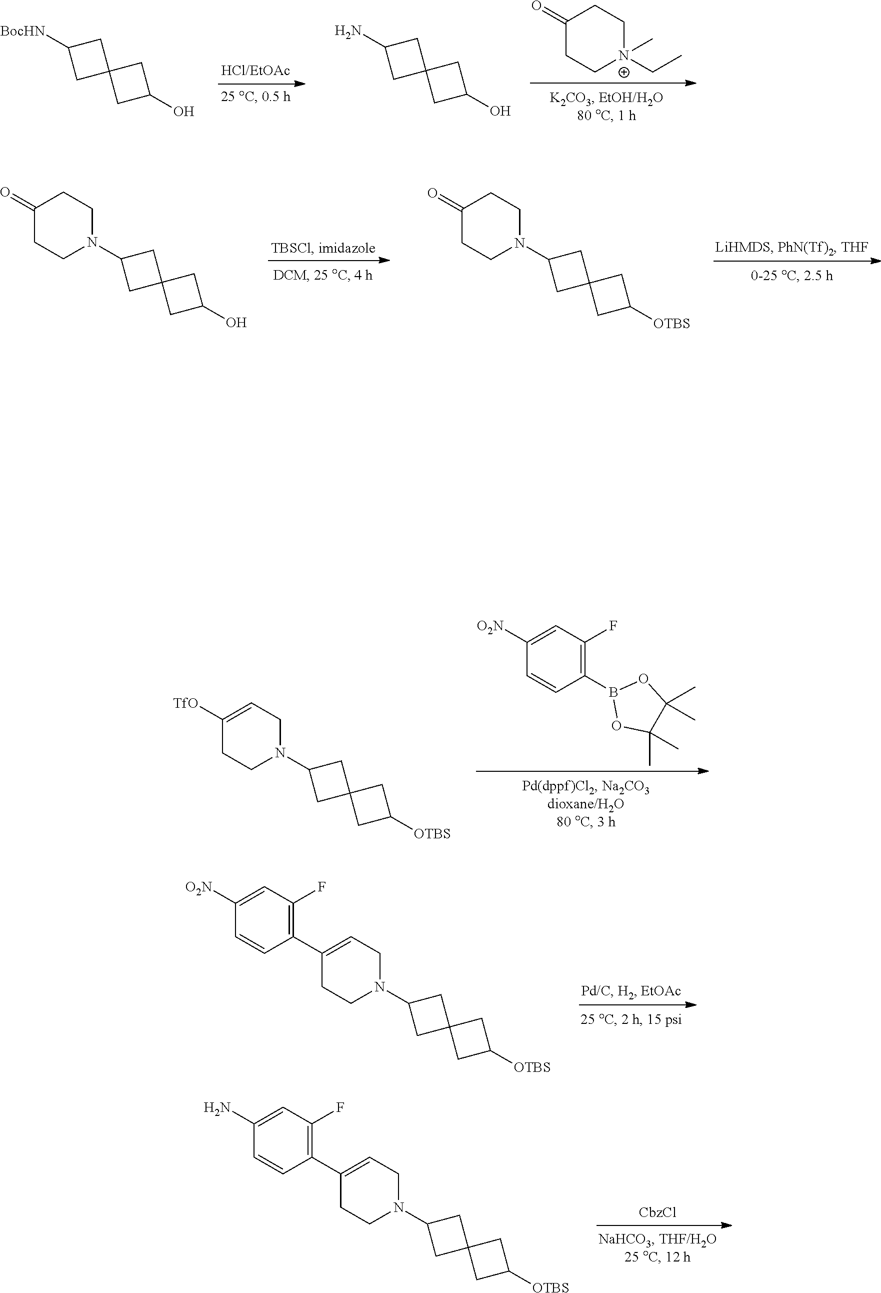

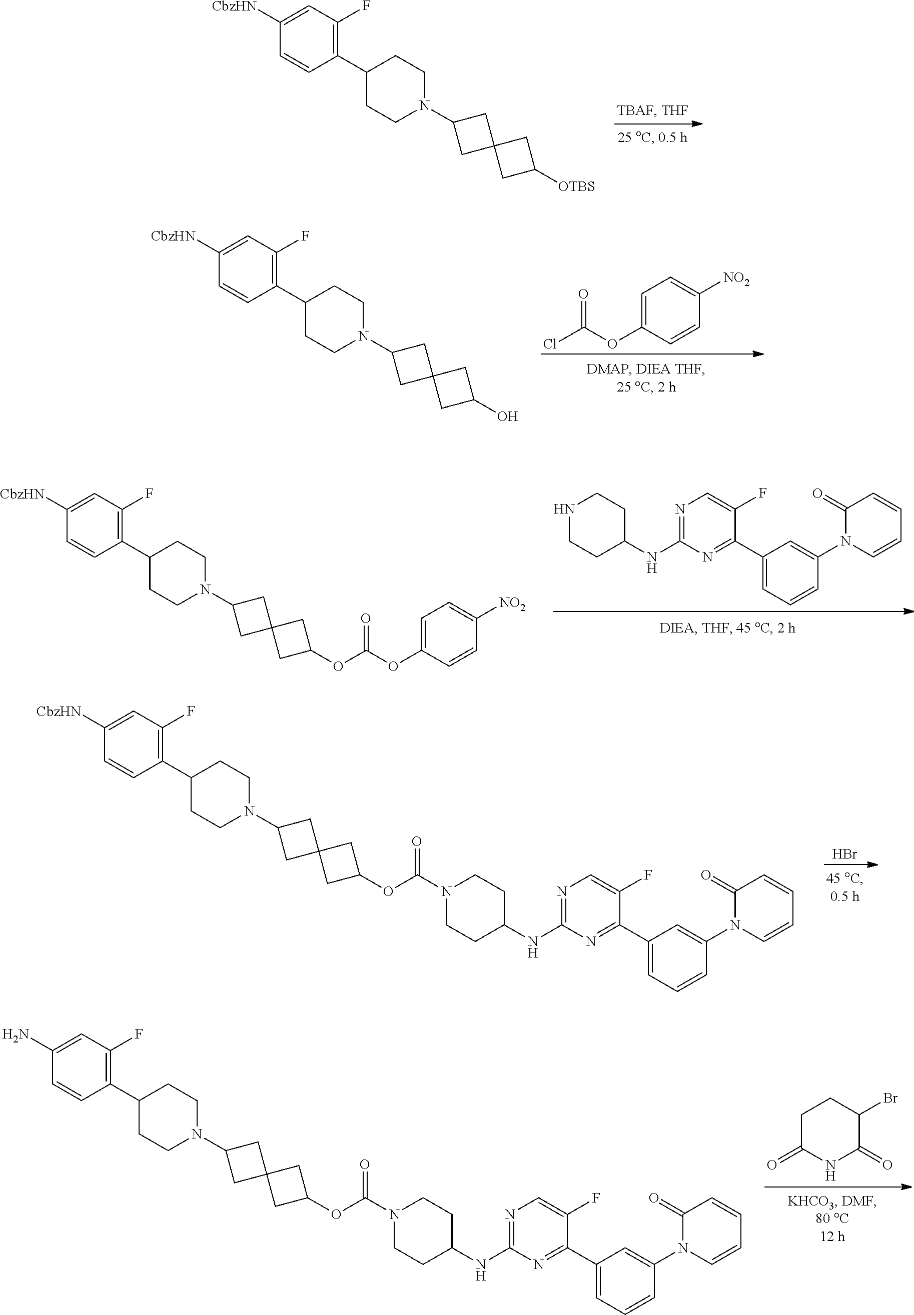

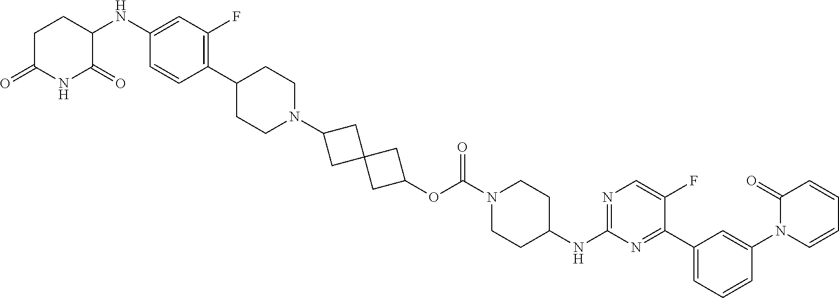 [SMILES: O=C1CCC(Nc2ccc(C3CCN(C4CC5(CC(OC(=O)N6CCC(Nc7ncc(F)c(-c8cccc(-n9ccccc9=O)c8)n7)CC6)C5)C4)CC3)c(F)c2)C(=O)N1]

Synthesis of 6-aminospiro[3.3]heptan-2-ol. A solution of tert-butyl (6-hydroxyspiro[3.3]heptan-2-yl)carbamate (6.50 g, 28.6 mmol, 1.00 eq) in HCl/EtOAc (30 mL) was stirred at 25 °C for 0.5 h. LCMS showed that tert-butyl (6-hydroxyspiro[3.3]heptan-2-yl)carbamate was consumed and the desired mass was detected. The mixture was concentrated under reduced pressure to give the title compound (4.60 g, crude, HCl) as a white solid. LCMS (ES⁺): m/z 128.3 [M+H]⁺.

Synthesis of 1-(6-hydroxyspiro[3.3]heptan-2-yl)piperidin-4-one. To a solution of 6-aminospiro[3.3]heptan-2-ol (4.60 g, 28.1 mmol, 1.00 eq, HCl) in EtOH (30 mL) and H₂O (10 mL) was added K₂CO₃ (7.77 g, 56.2 mmol, 2.00 eq) and 1-ethyl-1-methyl-4-oxopiperidin-1-ium (7.56 g, 28.1 mmol, 1.00 eq) was added at 25 °C, the mixture was stirred at 80 °C for 1 h. LCMS showed that 6-aminospiro[3.3]heptan-2-ol was consumed and the desired mass was detected. The mixture was concentrated under reduced pressure to remove EtOH. Then to the aqueous phase was added H₂O (50 mL) and the mixture was extracted with EtOAc (50 mL*3). The combined organic layers were dried over Na₂SO₄, filtered and the filtrate was concentrated under reduced pressure to give the title compound (4.60 g, crude) as a red oil. LCMS (ES⁺): m/z 210.2 [M+H]⁺.

Synthesis of 1-(6-((tert-butyldimethylsilyl)oxy)spiro[3.3]heptan-2-yl)piperidin-4-one. To a solution of 1-(6-hydroxyspiro[3.3]heptan-2-yl)piperidin-4-one (4.00 g, 19.1 mmol, 1.00 eq) in DCM (30 mL) were added TBSCl (5.76 g, 38.2 mmol, 4.70 mL, 2.00 eq) and imidazole (2.60 g, 38.2 mmol, 2.00 eq) at 25 °C, the mixture was stirred at 25 °C for 4 h. LCMS showed that 1-(6-hydroxyspiro[3.3]heptan-2-yl)piperidin-4-one was consumed and the desired mass was detected. To the mixture was added H₂O (30 mL) and the mixture was extracted with DCM (30 mL*3). The combined organic layers were dried over Na₂SO₄, filtered and the filtrate was concentrated under reduced pressure to give a residue. The residue was purified by MPLC (SiO₂, Petroleum ether/Ethyl acetate=1/0 to 3/1) to give the title compound (2.50 g, 7.73 mmol, 40.43% yield) as a yellow solid. LCMS (ES⁺): m/z 324.3 [M+H]⁺.

Synthesis of 1-(6-((tert-butyldimethylsilyl)oxy)spiro[3.3]heptan-2-yl)-1,2,3,6-tetrahydropyridin-4-yl trifluoromethanesulfonate. To a solution of 1-(6-((tert-butyldimethylsilyl)oxy)spiro[3.3]heptan-2-yl)piperidin-4-one (2.50 g, 7.73 mmol, 1.00 eq) in THF (30 mL) was added LiHMDS (1 M in THF, 11.6 mL, 1.50 eq) at 0 °C, the mixture was stirred at 0 °C for 30 min. Then 1,1,1-trifluoro-N-phenyl-N-(trifluoromethylsulfonyl)methanesulfonamide (4.14 g, 11.6 mmol, 1.50 eq) was added at 0 °C, the mixture was stirred at 25 °C for 2 h. TLC (Petroleum ether/Ethyl acetate=3/1) showed that 1-(6-((tert-butyldimethylsilyl)oxy)spiro[3.3]heptan-2-yl)piperidin-4-one was consumed and one new spot formed. The mixture was quenched by MeOH (10 ml) at 0 °C under N₂, then to the mixture was added H₂O (50 mL) and the mixture was extracted with EtOAc (30 mL*3). The combined organic layers were washed with brine (30 mL*3), dried over Na₂SO₄, filtered and the filtrate was concentrated under reduced pressure to give a residue. The residue was purified by MPLC (SiO₂, Petroleum ether/Ethyl acetate=1/0 to 3/1) to give the title compound (2.20 g, 4.83 mmol, 62.49% yield) as a yellow solid. The product couldn't detected by LCMS, it was confirmed by ¹H NMR (400 MHz, DMSO-d₆) δ=5.98 (br s, 1H), 4.20-4.09 (m, 1H), 3.23 (br s, 2H), 3.13-3.02 (m, 1H), 2.78 (br s, 2H), 2.42-2.29 (m, 2H), 2.18 (br s, 2H), 2.12-2.04 (m, 1H), 2.00-1.90 (m, 2H), 1.90-1.80 (m, 2H), 1.14 (dd, J=1.6, 7.0 Hz, 1H), 0.83 (d, J=2.1 Hz, 9H), 0.00 (d, J=2.0 Hz, 6H)

Synthesis of 1-(6-((tert-butyldimethylsilyl)oxy)spiro[3.3]heptan-2-yl)-4-(2-fluoro-4-nitrophenyl)-1,2,3,6-tetrahydropyridine. To a solution of 1-(6-((tert-butyldimethylsilyl)oxy)spiro[3.3]heptan-2-yl)-1,2,3,6-tetrahydropyridin-4-yl trifluoromethanesulfonate (2.20 g, 4.83 mmol, 1.00 eq) and 2-(2-fluoro-4-nitrophenyl)-4,4,5,5-tetramethyl-1,3,2-dioxaborolane (1.55 g, 5.79 mmol, 1.20 eq) in dioxane (30 mL) and H₂O (5 mL) were added Pd(dppf)Cl₂ (353 mg, 483 μmol, 0.100 eq) and Na₂CO₃ (1.02 g, 9.66 mmol, 2.00 eq) at 25 °C under N₂, the mixture was stirred at 80 °C for 3 h under N₂. LCMS showed that the desired mass was detected. To the mixture was added H₂O (20 mL) and the mixture was extracted with EtOAc (20 mL*3). The combined organic layers were dried over Na₂SO₄, filtered and the filtrate was concentrated under reduced pressure to give a residue. The residue was purified by MPLC (SiO₂, Petroleum ether/Ethyl acetate=1/0 to 3/1) to give the title compound (1.20 g, 2.69 mmol, 55.64% yield) as a red solid. LCMS (ES⁺): m/z 447.3 [M+H]⁺.

Synthesis of 4-(1-(6-((tert-butyldimethylsilyl)oxy)spiro[3.3]heptan-2-yl)piperidin-4-yl)-3-fluoroaniline. To 10% Pd/C (2.00 g, 50% purity) in EtOAc (20 mL) was added 1-(6-((tert-butyldimethylsilyl)oxy)spiro[3.3]heptan-2-yl)-4-(2-fluoro-4-nitrophenyl)-1,2,3,6-tetrahydropyridine (1.20 g, 2.69 mmol, 1.00 eq) at 25 °C under N₂. The mixture was purged and degassed with H₂ for three times. The mixture was stirred at 25 °C for 2 h under H₂ (15 psi). LCMS showed that the 1-(6-((tert-butyldimethylsilyl)oxy)spiro[3.3]heptan-2-yl)-4-(2-fluoro-4-nitrophenyl)-1,2,3,6-tetrahydropyridine was consumed and the desired mass was detected. The mixture was filtered and the filtrate was concentrated under reduced pressure to give the title compound (1.00 g, crude) as a white solid. LCMS (ES⁺): m/z 419.2 [M+H]⁺.

Synthesis of benzyl (4-(1-(6-((tert-butyldimethylsilyl)oxy)spiro[3.3]heptan-2-yl)piperidin-4-yl)-3-fluorophenyl)carbamate. To a solution of 4-(1-(6-((tert-butyldimethylsilyl)oxy)spiro[3.3]heptan-2-yl)piperidin-4-yl)-3-fluoroaniline (1.00 g, 2.39 mmol, 1.00 eq) in THE (30 mL) and H₂O (10 mL) were added CbzCl (489 mg, 2.87 mmol, 409 μL, 1.20 eq) and NaHCO₃ (401 mg, 4.78 mmol, 186 μL, 2.00 eq) at 25 °C, the mixture was stirred at 25 °C for 12 h. LCMS showed that the 4-(1-(6-((tert-butyldimethylsilyl)oxy)spiro[3.3]heptan-2-yl)piperidin-4-yl)-3-fluoroaniline was consumed and the desired mass was detected. To the mixture was added H₂O (50 mL) and the mixture was extracted with EtOAc (50 mL*3). The combined organic layers were dried over Na₂SO₄, filtered and the filtrate was concentrated under reduced pressure to give the title compound (1.50 g, crude) as a white solid. LCMS (ES⁺): m/z 553.4 [M+H]⁺.

Synthesis of benzyl (3-fluoro-4-(1-(6-hydroxyspiro[3.3]heptan-2-yl)piperidin-4-yl)phenyl)carbamate. To a solution of benzyl (4-(1-(6-((tert-butyldimethylsilyl)oxy)spiro[3.3]heptan-2-yl)piperidin-4-yl)-3-fluorophenyl)carbamate (1.50 g, 2.71 mmol, 1.00 eq) in THF (30 mL) was added TBAF (1 M in THF, 3.26 mL, 1.20 eq) at 25 °C, the mixture was stirred at 25 °C for 0.5 h. LCMS showed that the benzyl (4-(1-(6-((tert-butyldimethylsilyl)oxy)spiro[3.3]heptan-2-yl)piperidin-4-yl)-3-fluorophenyl)carbamate was consumed and the desired mass was detected. The mixture was concentrated under reduced pressure to give a residue. The mixture was purified by column chromatography (SiO₂, EtOAc/MeOH=1/0 to 20/1) to give the title compound (1.00 g, 2.28 mmol, 84.04% yield) as a white solid. LCMS (ES⁺): m/z 439.3 [M+H]⁺.

Synthesis of benzyl (3-fluoro-4-(1-(6-(((4-nitrophenoxy)carbonyl)oxy)spiro[3.3]heptan-2-yl)piperidin-4-yl)phenyl)carbamate. To a solution of benzyl (3-fluoro-4-(1-(6-hydroxyspiro[3.3]heptan-2-yl)piperidin-4-yl)phenyl)carbamate (1.00 g, 2.28 mmol, 1.00 eq) and 4-nitrophenyl carbonochloridate (689 mg, 3.42 mmol, 1.50 eq) in THF (20 mL) were added DMAP (279 mg, 2.28 mmol, 1.00 eq) and DIEA (589 mg, 4.56 mmol, 794 μL, 2.00 eq) at 25 °C, the mixture was stirred at 25 °C for 2 h. LCMS showed that the benzyl (3-fluoro-4-(1-(6-hydroxyspiro[3.3]heptan-2-yl)piperidin-4-yl)phenyl)carbamate was consumed and the desired mass was detected. To the mixture was added H₂O (20 mL) and the mixture was extracted with EtOAc (20 mL*3). The combined organic layers were dried over Na₂SO₄, filtered and the filtrate was concentrated under reduced pressure to give the crude product. The mixture was purified by MPLC (SiO2, Petroleum ether/Ethyl acetate=1/0 to 0/1) to give the title compound (700 mg, 1.16 mmol, 50.85% yield) as a yellow solid. LCMS (ES⁺): m/z 604.2 [M+H]⁺.

Synthesis of 6-(4-(4-(((benzyloxy)carbonyl)amino)-2-fluorophenyl)piperidin-1-yl)spiro[3.3]heptan-2-yl 4-((5-fluoro-4-(3-(2-oxopyridin-1 (2H)-yl)phenyl)pyrimidin-2-yl)amino)piperidine-1-carboxylate. To a solution of benzyl (3-fluoro-4-(1-(6-(((4-nitrophenoxy)carbonyl)oxy)spiro[3.3]heptan-2-yl)piperidin-4-yl)phenyl)carbamate (300 mg, 497 μmol, 1.00 eq) and 1-(3-(5-fluoro-2-(piperidin-4-ylamino)pyrimidin-4-yl)phenyl)pyridin-2 (1H)-one (238 mg, 497 μmol, 1.00 eq, TFA) in THF (3 mL) was added DIEA (128 mg, 994 mol, 173 μL, 2.00 eq) at 25 °C, the mixture was stirred at 45 °C for 2 h. LCMS showed that the benzyl (3-fluoro-4-(1-(6-(((4-nitrophenoxy)carbonyl)oxy)spiro[3.3]heptan-2-yl)piperidin-4-yl)phenyl)carbamate was consumed and the desired mass was detected. To the mixture was added H₂O (10 mL) and the mixture was extracted with EtOAc (10 mL*3). The combined organic layers were dried over Na₂SO₄, filtered and the filtrate was concentrated under reduced pressure to give a residue. The residue was purified by prep-TLC (SiO₂, EtOAc/MeOH=20/1) to give the title compound (300 mg, 362 μmol, 72.73% yield) as a white solid. LCMS (ES⁺): m/z 830.3 [M+H]⁺.

Synthesis of 6-(4-(4-amino-2-fluorophenyl)piperidin-1-yl)spiro[3.3]heptan-2-yl 4-((5-fluoro-4-(3-(2-oxopyridin-1 (2H)-yl)phenyl)pyrimidin-2-yl)amino)piperidine-1-carboxylate. A solution of 4-((5-fluoro-4-(3-(2-oxopyridin-1 (2H)-yl)phenyl)pyrimidin-2-yl)amino)piperidine-1-carboxylate (200 mg, 241 μmol, 1.00 eq) in HBr (3 mL in H₂O) was added at 45 °C for 0.5 h. LCMS showed that the 4-((5-fluoro-4-(3-(2-oxopyridin-1 (2H)-yl)phenyl)pyrimidin-2-yl)amino)piperidine-1-carboxylate was consumed and the desired mass was detected. To the mixture was added NaHCO₃ solution (10 mL) and the mixture was extracted with EtOAc (10 mL*3). The combined organic layers were dried over Na₂SO₄, filtered and the filtrate was concentrated under reduced pressure to give a residue. The residue was purified by prep-TLC (SiO₂, EtOAc/MeOH=20/1) to give the title compound (150 mg, 216 μmol, 89.46% yield) as a white solid. LCMS (ES⁺): m/z 696.5 [M+H]⁺.

Synthesis of 6-(4-(4-((2,6-dioxopiperidin-3-yl)amino)-2-fluorophenyl)piperidin-1-yl)spiro[3.3]heptan-2-yl 4-((5-fluoro-4-(3-(2-oxopyridin-1 (2H)-yl)phenyl)pyrimidin-2-yl)amino)piperidine-1-carboxylate (Compound 633). To a solution of 6-(4-(4-amino-2-fluorophenyl)piperidin-1-yl)spiro[3.3]heptan-2-yl 4-((5-fluoro-4-(3-(2-oxopyridin-1 (2H)-yl)phenyl)pyrimidin-2-yl)amino)piperidine-1-carboxylate (60.0 mg, 86.2 μmol, 1.00 eq) in DMF (3 mL) were added KHCO₃ (86.3 mg, 862 μmol, 10.0 eq) and 3-bromopiperidine-2,6-dione (99.3 mg, 517 μmol, 6.00 eq) at 25 °C, the mixture was stirred at 80 °C for 12 h. LCMS showed that the 6-(4-(4-amino-2-fluorophenyl)piperidin-1-yl)spiro[3.3]heptan-2-yl 4-((5-fluoro-4-(3-(2-oxopyridin-1 (2H)-yl)phenyl)pyrimidin-2-yl)amino)piperidine-1-carboxylate was consumed and the desired mass was detected. The reaction was filtered and the filtrate was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 33%-57% B over 8.0 min) to give the title compound (Compound 633) (3.00 mg, 3.52 μmol, 4.09% yield, 94.77% purity) as a pale solid. LCMS (ES⁺): m/z 807.3 [M+H]⁺.

### Example 625. Synthesis of trans-[4-[[5-fluoro-4-[3-(2-oxo-1-pyridyl)phenyl]pyrimidin-2-yl]amino]cyclohexyl]4-[1-[4-[(2,6-dioxo-3-piperidyl)amino]-2-fluoro-phenyl]-3-fluoro-4-piperidyl]piperazine-1-carboxylate (Compound 668)

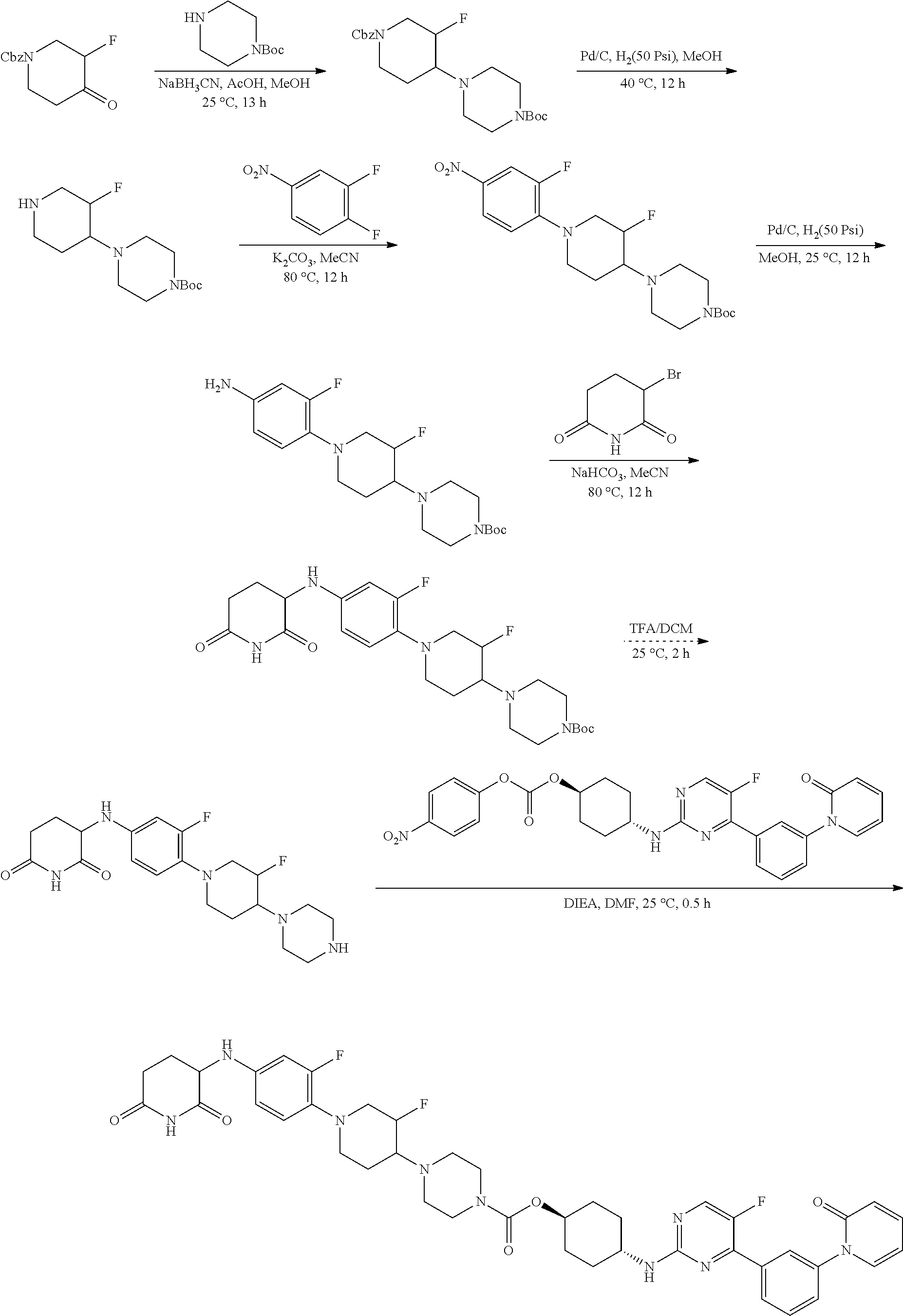

Synthesis of tert-butyl 4-(1-benzyloxycarbonyl-3-fluoro-4-piperidyl)piperazine-1-carboxylate. The reaction was set up in two batches. To a solution of benzyl 3-fluoro-4-oxo-piperidine-1-carboxylate (5.00 g, 19.9 mmol, 1.00 eq) in MeOH (50 mL) was added AcOH (2.39 g, 39.8 mmol, 2.28 mL, 2.00 eq), tert-butyl piperazine-1-carboxylate (18.5 g, 99.5 mmol, 5.00 eq) at 25 °C. After addition, the mixture was stirred at this temperature for 1 h, and then NaBH₃CN (2.50 g, 39.8 mmol, 2.00 eq) was added the mixture at 25 °C. The resulting mixture was stirred at 25 °C for 12 h. LC-MS showed the desired mass was detected. The reaction mixture was quenched by ice water 100 mL at 0 °C, and then extracted with EtOAc (100 mL*3). The combined organic layers were washed with brine (50 mL), dried over Na₂SO₄, filtered and concentrated under reduced pressure. The residue was purified by column chromatography (SiO2, Petroleum ether:Ethyl acetate=1:0 to 3:1) to give the title compound (150 mg, crude, HCl) as a yellow oil. LCMS (ES⁺): m/z 422.2 [M+H]⁺.

Synthesis of tert-butyl 4-(3-fluoro-4-piperidyl)piperazine-1-carboxylate. To a solution of tert-butyl 4-(1-benzyloxycarbonyl-3-fluoro-4-piperidyl)piperazine-1-carboxylate (3.00 g, 7.12 mmol, 1.00 eq) in MeOH (50 mL) was added 10% Pd/C (2.00 g, 9.40 mmol, 50% purity, 1.32 eq) at 25 °C under N₂ atmosphere. The suspension was degassed and purged with H₂ for 3 times. The mixture was stirred under H₂ (50 Psi) at 40 °C for 12 h. LC-MS showed the desired mass was detected. The reaction mixture was filtered and concentrated under reduced pressure to give the title compound (2.50 g, crude) as a yellow solid. LCMS (ES⁺): m/z 288.3 [M+H]⁺.

Synthesis of tert-butyl 4-[3-fluoro-1-(2-fluoro-4-nitro-phenyl)-4-piperidyl]piperazine-1-carboxylate. To a solution of tert-butyl 4-(3-fluoro-4-piperidyl)piperazine-1-carboxylate (2.00 g, 6.96 mmol, 1.00 eq) in MeCN (2 mL) was added K₂CO₃ (1.92 g, 13.9 mmol, 2.00 eq) and 1,2-difluoro-4-nitro-benzene (2.21 g, 13.9 mmol, 1.54 mL, 2.00 eq) at 25 °C. The mixture was stirred at 80 °C for 12 h. LC-MS showed the desired mass was detected. The reaction mixture was quenched by ice water 20 mL at 0 °C, and then extracted with EtOAc (20 mL*3). The combined organic layers were washed with brine (50 mL), dried over Na₂SO₄, filtered and concentrated under reduced pressure. The residue was purified by column chromatography (SiO₂, Petroleum ether:Ethyl acetate=1:0 to 1:1) to give the title compound (1.80 g, 4.22 mmol, 60.6% yield) as a yellow oil. LCMS (ES⁺): m/z 427.3 [M+H]⁺.

Synthesis of tert-butyl 4-[1-(4-amino-2-fluoro-phenyl)-3-fluoro-4-piperidyl]piperazine-1-carboxylate. A mixture of tert-butyl 4-[3-fluoro-1-(2-fluoro-4-nitro-phenyl)-4-piperidyl]piperazine-1-carboxylate (1.80 g, 4.22 mmol, 1.00 eq), 10% Pd/C (1.80 g, 8.46 mmol, 50.0% purity, 2.00 eq) in MeOH (2 mL) was stirred at 25 °C for 12 h under H₂ (15 Psi) atmosphere. LC-MS showed the desired mass was detected. The reaction mixture was filtered and concentrated under reduced pressure to give the title compound (1.20 g, crude) as a yellow solid. LCMS (ES⁺): m/z 397.3 [M+H]⁺.

Synthesis of tert-butyl 4-(1-(4-((2,6-dioxopiperidin-3-yl)amino)-2-fluorophenyl)-3-fluoropiperidin-4-yl)piperazine-1-carboxylate. To a solution of cis-tert-butyl 4-[1-(4-amino-2-fluoro-phenyl)-3-fluoro-4-piperidyl]piperazine-1-carboxylate (600 mg, 1.51 mmol, 1.00 eq) in MeCN (2 mL) was added NaHCO₃ (381 mg, 4.54 mmol, 176 μL, 3.00 eq) and 3-bromopiperidine-2,6-dione (871 mg, 4.54 mmol, 3.00 eq) at 25 °C. The mixture was stirred at 80 °C for 12 h. LC-MS showed the desired mass was detected. The reaction mixture was quenched by ice water 20 mL at 0 °C, and then extracted with EtOAc (20 mL*3). The combined organic layers were washed with brine (50 mL), dried over Na₂SO₄, filtered and concentrated under reduced pressure. The residue was purified by column chromatography (SiO₂, Petroleum ether:Ethyl acetate=1:0 to 3:1) to give the title compound (250 mg, 492 μmol, 16.2% yield) as a blue solid. LCMS (ES⁺): m/z 508.3 [M+H]⁺

Synthesis of 3-[3-fluoro-4-(3-fluoro-4-piperazin-1-yl-1-piperidyl)anilino]piperidine-2,6-dione. A mixture of tert-butyl 4-[1-[4-[(2,6-dioxo-3-piperidyl)amino]-2-fluoro-phenyl]-3-fluoro-4-piperidyl]piperazine-1-carboxylate (250 mg, 492 μmol, 1.00 eq) and TFA (767 mg, 6.73 mmol, 0.5 mL, 13.6 eq) in DCM (2 mL) was stirred at 25 °C for 2 h. LC-MS showed the desired mass was detected. The reaction mixture was concentrated under reduced pressure to give the title compound (200 mg, crude, TFA) as a yellow oil. LCMS (ES⁺): m/z 408.2 [M+H]⁺.

Synthesis of trans-[4-[[5-fluoro-4-[3-(2-oxo-1-pyridyl)phenyl]pyrimidin-2-yl]amino]cyclohexyl]4-[1-[4-[(2,6-dioxo-3-piperidyl)amino]-2-fluoro-phenyl]-3-fluoro-4-piperidyl]piperazine-1-carboxylate (Compound 668). To a solution of 3-[3-fluoro-4-(3-fluoro-4-piperazin-1-yl-1-piperidyl)anilino]piperidine-2,6-dione (190 mg, 466 μmol, 1.00 eq) in DMF (0.5 mL) was added DIEA (120 mg, 932 μmol, 162 μL, 2.00 eq) and trans-4-((5-fluoro-4-(3-(2-oxopyridin-1 (2H)-yl)phenyl)pyrimidin-2-yl)amino)cyclohexyl (4-nitrophenyl) carbonate (254 mg, 466 μmol, 1.00 eq). The mixture was stirred at 25 °C for 0.5 h. LC-MS showed the desired mass was detected. The reaction mixture was quenched by addition H₂O 3 mL, and then extracted with EtOAc (3 mL*3). The combined organic layers were dried over Na₂SO₄, filtered and concentrated under reduced pressure. The residue was purified by prep-HPLC (column: Phenomenex luna C18 100*40 mm*5 um; mobile phase: [H₂O (0.04% HCl)-ACN]; gradient: 15%-35% B over 8.0 min) to give the title compound (Compound 668) (121 mg, 143 μmol, 30.7% yield, 95.8% purity) as a brown solid. LCMS (ES⁺): m/z 814.4[M+H]⁺.

### Example 626. Synthesis of trans-4-((5-fluoro-4-(3-(2-oxopyridin-1 (2H)-yl)phenyl)pyrimidin-2-yl)amino)cyclohexyl 4-(4-((2,6-dioxopiperidin-3-yl)amino)-2-fluorophenyl)-3′-fluoro-[1,4′-bipiperidine]-1′-carboxylate (Compound 695)

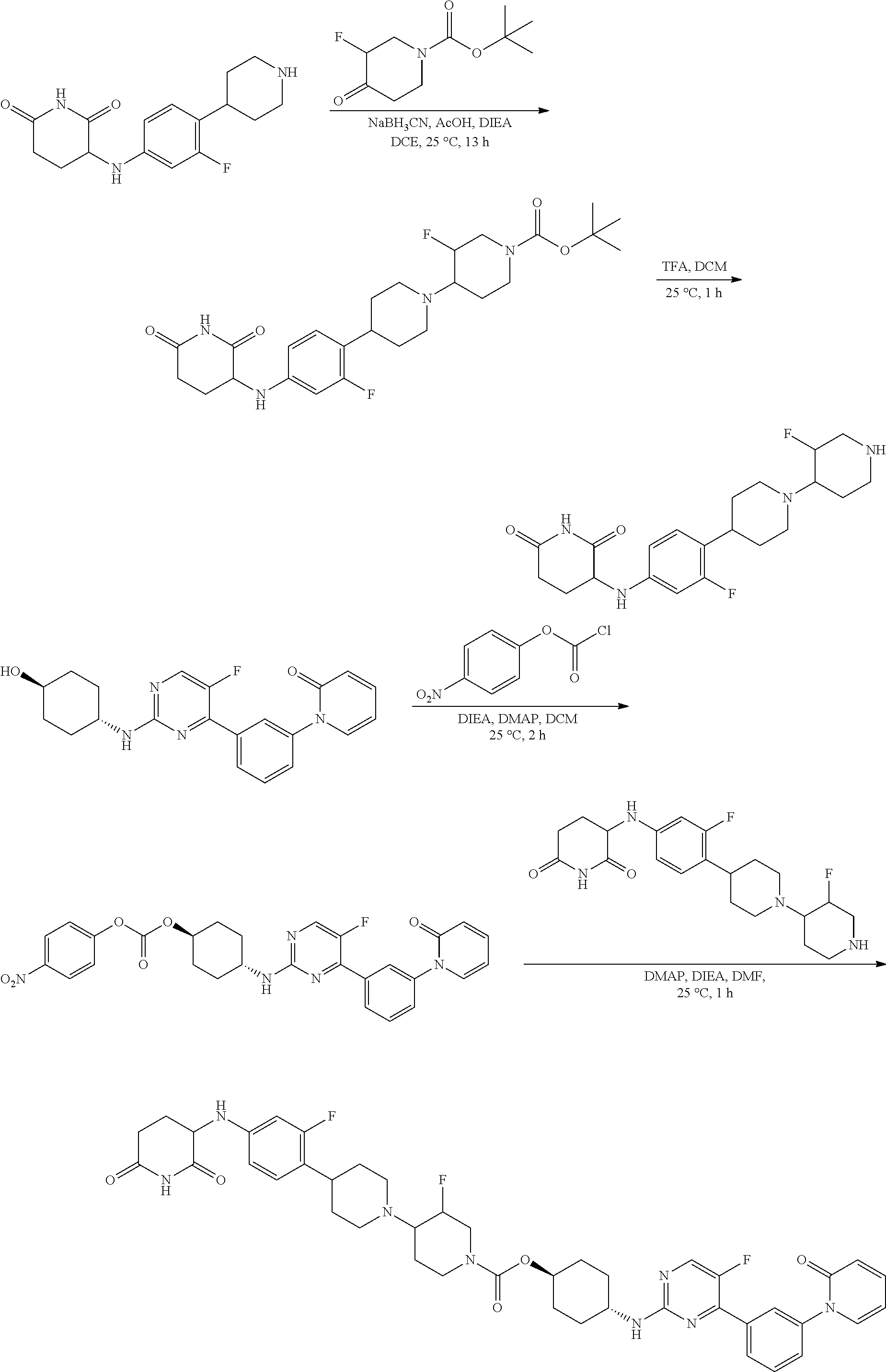

Synthesis of tert-butyl cis-4-(4-((2,6-dioxopiperidin-3-yl)amino)-2-fluorophenyl)-3′-fluoro-[1,4′-bipiperidine]-1′-carboxylate. To a solution of 3-[3-fluoro-4-(4-piperidyl)anilino]piperidine-2,6-dione (1.00 g, 2.38 mmol, 1.00 eq, TFA) in DCE (15 mL) was added DIEA (924 mg, 7.15 mmol, 1.25 mL, 3.00 eq) at 25 °C, the mixture was stirred at 25 °C for 0.5 h. Then AcOH (715 mg, 11.9 mmol, 682 μL, 5.00 eq) and tert-butyl 3-fluoro-4-oxo-piperidine-1-carboxylate (518 mg, 2.38 mmol, 1.00 eq) was added at 25 °C, the mixture was stirred at 25 °C for 0.5 h. Then NaBH3CN (749 mg, 11.9 mmol, 5.00 eq) at 25 °C. The mixture was stirred at 25 °C for 12 h. LC-MS showed the desired mass was detected. The reaction mixture was diluted with water 15 mL and extracted with DCM (15 mL*3). The combined organic layers dried over Na₂SO₄, filtered and concentrated under reduced pressure. The residue was purified by column chromatography (SiO₂, Petroleum ether:Ethyl acetate=1:0 to 0:1) to give the title compound (1.00 g, 1.58 mmol, 66.23% yield, 80% purity) as a yellow oil. LCMS (ES⁺): m/z 507.3 [M+H]⁺.

Synthesis of 3-((3-fluoro-4-(cis-3′-fluoro-[1,4′-bipiperidin]-4-yl)phenyl)amino)piperidine-2,6-dione. To a solution of tert-butyl cis-4-(4-((2,6-dioxopiperidin-3-yl)amino)-2-fluorophenyl)-3′-fluoro-[1,4′-bipiperidine]-1′-carboxylate (650 mg, 1.28 mmol, 1.00 eq) in DCM (5 mL) was added TFA (3.84 g, 33.6 mmol, 2.5 mL, 26.2 eq) at 25 °C. The mixture was stirred at 25 °C for 1 h. LC-MS showed the desired mass was detected. The reaction mixture was concentrated under reduced pressure to give the title compound (650 mg, crude, TFA) as brown oil. LCMS (ES⁺): m/z 407.4 [M+H]⁺.

Synthesis of trans-4-((5-fluoro-4-(3-(2-oxopyridin-1 (2H)-yl)phenyl)pyrimidin-2-yl)amino)cyclohexyl (4-nitrophenyl) carbonate. To a solution of trans-1-(3-(5-fluoro-2-((4-hydroxycyclohexyl)amino)pyrimidin-4-yl)phenyl)pyridin-2 (1H)-one (2.20 g, 5.78 mmol, 1.00 eq) and DMAP (635 mg, 5.20 mmol, 0.900 eq) in DCM (30 mL) was added DIEA (2.84 g, 21.9 mmol, 3.83 mL, 3.80 eq) and 4-nitrophenyl carbonochloridate (2.21 g, 10.9 mmol, 1.90 eq) at 25 °C. The mixture was stirred at 25 °C for 2 h. LC-MS showed the desired mass was detected. The reaction mixture was diluted with water 150 mL and extracted with EtOAc (150 mL*3). The combined organic layers dried over Na₂SO₄, filtered and concentrated under reduced pressure. The residue was purified by column chromatography (SiO₂, Petroleum ether:Ethyl acetate=1:0 to 1:1) to give the title compound (1.10 g, 1.63 mmol, 28.2% yield, 80.8% purity) as a yellow solid. LCMS (ES⁺): m/z 546.2 [M+H]⁺.

Synthesis of trans-4-((5-fluoro-4-(3-(2-oxopyridin-1 (2H)-yl)phenyl)pyrimidin-2-yl)amino)cyclohexyl 4-(4-((2,6-dioxopiperidin-3-yl)amino)-2-fluorophenyl)-3′-fluoro-[1,4′-bipiperidine]-1′-carboxylate (Compound 695) To a solution of trans-4-((5-fluoro-4-(3-(2-oxopyridin-1 (2H)-yl)phenyl)pyrimidin-2-yl)amino)cyclohexyl (4-nitrophenyl) carbonate (200 mg, 366 μmol, 1.00 eq), 3-((3-fluoro-4-(3′-fluoro-[1,4′-bipiperidin]-4-yl)phenyl)amino)piperidine-2,6-dione (190 mg, 366 μmol, 1.00 eq, TFA) in DMF (3 mL) was added DIEA (118 mg, 91.0 μmol, 159 μL, 2.50 eq) and DMAP (44.7 mg, 366 μmol, 1.00 eq) at 25 °C. The mixture was stirred at 25 °C for 1 h. LC-MS showed the desired mass was detected. The reaction mixture was concentrated under reduced pressure. The residue was purified by prep-HPLC (column: Waters Xbridge BEH C18 100*30 mm*10 um; mobile phase: [H₂O (10 mM NH₄HCO₃)-ACN]; gradient: 36%-66% B over 8.0 min). The residue was purified by prep-HPLC (column: Phenomenex Luna C18 100*30 mm*3 um; mobile phase: [1H₂O (0.2% FA)-ACN]; gradient: 15%-45% B over 8.0 min) to give the title compound (Compound 695) (11.6 mg, 14.2 μmol, 3.89% yield, 100% purity) as a white solid. LCMS (ES⁺): m/z 813.3 [M+H]⁺.

### Example 627. Synthesis of trans-4-((5-fluoro-4-(3-(2-oxopyridin-1 (2H)-yl)phenyl)pyrimidin-2-yl)amino)cyclohexyl 4-(4-((2,6-dioxopiperidin-3-yl)amino)-2-fluorophenyl)-3′,3′-difluoro-[1,4′-bipiperidine]-1′-carboxylate (Compound 648)

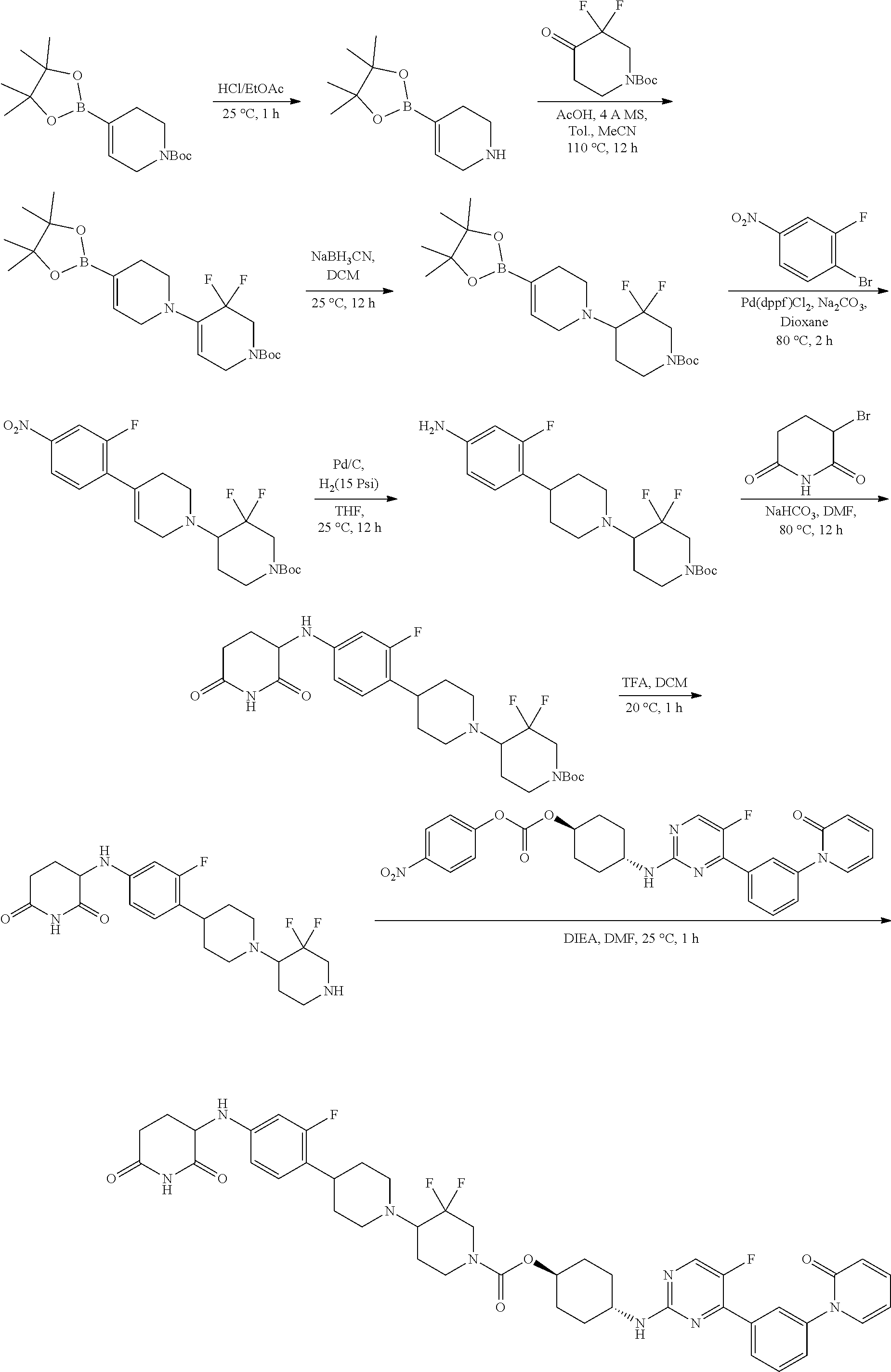

Synthesis of 4-(4,4,5,5-tetramethyl-1,3,2-dioxaborolan-2-yl)-1,2,3,6-tetrahydropyridine. To a solution of tert-butyl 4-(4,4,5,5-tetramethyl-1,3,2-dioxaborolan-2-yl)-3,6-dihydropyridine-1 (2H)-carboxylate (20.0 g, 64.6 mmol, 1.00 eq) in HCl/EtOAc (4 M, 200 mL, 12.3 eq) was stirred at 25 °C for 1 h. LC-MS showed the desired mass was detected. The reaction mixture was concentrated under high vacuum to give the title compound (18.0 g, crude) as a white solid. LCMS (ES⁺): m/z 210.2 [M+H]⁺.

Synthesis of tert-butyl 3′,3′-difluoro-4-(4,4,5,5-tetramethyl-1,3,2-dioxaborolan-2-yl)-3,3′,6,6′-tetrahydro-2H-[1,4′-bipyridine]-1′(2′H)-carboxylate. To the 4-(4,4,5,5-tetramethyl-1,3,2-dioxaborolan-2-yl)-1,2,3,6-tetrahydropyridine (10.0 g, 47.8 mmol, 1.00 eq) in ACN (50 mL) and toluene (50 mL) was added DIEA (12.3 g, 95.6 mmol, 16.6 mL, 2.00 eq) at 25 °C. The resulting reaction mixture was stirred for 1 h. Then the reaction mixture was added tert-butyl 3,3-difluoro-4-oxo-piperidine-1-carboxylate (13.5 g, 57.3 mmol, 1.20 eq), AcOH (5.74 g, 95.6 mmol, 5.48 mL, 2.00 eq), Molecular Sieves (1.00 g) was added to the mixture. the reaction mixture was stirred at 110 °C for 11 h. LC-MS showed the desired mass was detected. The reaction mixture was concentrated under high vacuum to give the title compound (10.0 g, crude) as a yellow solid. LCMS (ES⁺): m/z 427.2 [M+H]⁺.

Synthesis of tert-butyl 3,3-difluoro-4-(4-(4,4,5,5-tetramethyl-1,3,2-dioxaborolan-2-yl)-3,6-dihydropyridin-1 (2H)-yl)piperidine-1-carboxylate To a solution of tert-butyl 3,3-difluoro-4-[4-(4,4,5,5-tetramethyl-1,3,2-dioxaborolan-2-yl)-3,6-dihydro-2H-pyridin-1-yl]-2,6-dihydropyridine-1-carboxylate (10.0 g, 23.4 mmol, 1.00 eq) in DCM (200 mL) was added NaBH₃CN (2.95 g, 46.9 mmol, 2.00 eq) at 25 °C. The mixture was stirred at 25 °C for 12 h. LC-MS showed the desired mass was detected. The reaction mixture was quenched by addition H₂O 100 mL, and then diluted with H₂O 100 mL and extracted with DCM (100 mL*3). The combined organic layers were washed with sat.aq. NaCl (10 mL), dried over Na₂SO₄, filtered and concentrated under reduced pressure to give a residue. The residue was purified by column chromatography (SiO₂, Petroleum ether:Ethyl acetate=1:0 to 3:1) to give the title compound (15.0 g, 17.5 mmol, 74.9% yield, 50.1% purity) as a yellow oil. LCMS (ES⁺): m/z 429.3 [M+H]⁺.

Synthesis of tert-butyl 3,3-difluoro-4-(4-(2-fluoro-4-nitrophenyl)-3,6-dihydropyridin-1 (2H)-yl)piperidine-1-carboxylate. A mixture of tert-butyl 3,3-difluoro-4-(4-(4,4,5,5-tetramethyl-1,3,2-dioxaborolan-2-yl)-3,6-dihydropyridin-1 (2H)-yl)piperidine-1-carboxylate (6.00 g, 14.0 mmol, 1.00 eq), 1-bromo-2-fluoro-4-nitro-benzene (1.54 g, 7.00 mmol, 0.500 eq), Na₂CO₃ (4.45 g, 42.0 mmol, 3.00 eq), Pd(dppf)Cl₂ (1.02 g, 1.40 mmol, 0.100 eq) in 1,4-dioxane (60 mL) was degassed and purged with N₂ at 25 °C for 3 times, and then the mixture was stirred at 80 °C for 2 h under N₂ atmosphere. LC-MS showed the desired mass was detected. The reaction mixture was quenched by addition H₂O 50 mL, and then diluted with H₂O 50 mL and extracted with EtOAc (50 mL*3). The combined organic layers were washed with sat.aq. NaCl (10 mL), dried over Na₂SO₄, filtered and concentrated under reduced pressure to give a residue. The residue was purified by column chromatography (SiO₂, Petroleum ether:Ethyl acetate=1:0 to 3:1) to give the title compound (6.00 g, 12.6 mmol, 90.5% yield, 93.3% purity) as a yellow oil. LCMS (ES⁺): m/z 442.3 [M+H]⁺.

Synthesis of tert-butyl 4-(4-amino-2-fluorophenyl)-3,3′-difluoro-[1,4′-bipiperidine]-1′-carboxylate. To a solution of tert-butyl 3,3-difluoro-4-(4-(2-fluoro-4-nitrophenyl)-3,6-dihydropyridin-1 (2H)-yl)piperidine-1-carboxylate (6.00 g, 13.5 mmol, 1.00 eq) in THF (60 mL) was added 10% Pd/C (3.00 g, 14.1 mmol, 50% purity, 1.04 eq) under N₂ at 25 °C. The suspension was degassed under vacuum and purged with H₂ several times. The mixture was stirred under H₂ (15 psi) at 25 °C for 12 h. LC-MS showed the desired mass was detected. The reaction mixture was filtered and the filtrate concentrated under high vacuum to give the title compound (6.00 g, crude) as a yellow solid. LCMS (ES⁺): m/z 414.2 [M+H]⁺.

Synthesis of tert-butyl 4-(4-((2,6-dioxopiperidin-3-yl)amino)-2-fluorophenyl)-3′,3′-difluoro-[1,4′-bipiperidine]-1′-carboxylate. To a solution of tert-butyl 4-(4-amino-2-fluorophenyl)-3′,3′-difluoro-[1,4′-bipiperidine]-1′-carboxylate (2.00 g, 4.84 mmol, 1.00 eq) in DMF (20 mL) was added 3-bromopiperidine-2,6-dione (2.79 g, 14.5 mmol, 3.00 eq), NaHCO₃ (1.22 g, 14.5 mmol, 564 μL, 3.00 eq) at 25 °C. The mixture was stirred at 80 °C for 12 h. LC-MS showed the desired mass was detected. The reaction mixture was quenched by addition H₂O 50 mL, and then diluted with H₂O 20 mL and extracted with EtOAc (50 mL*3). The combined organic layers were washed with sat.aq. NaCl (10 mL), dried over Na₂SO₄, filtered and concentrated under reduced pressure to give a residue. The residue was purified by column chromatography (SiO₂, PE:THF=1:0 to 0:1) to give the title compound (1.50 g, 2.70 mmol, 55.7% yield, 94.3% purity) as a green oil. LCMS (ES⁺): m/z 525.3 [M+H]⁺.

Synthesis of 3-((4-(3′,3′-difluoro-[1,4′-bipiperidin]-4-yl)-3-fluorophenyl)amino)piperidine-2,6-dione. To a solution of tert-butyl 4-(4-((2,6-dioxopiperidin-3-yl)amino)-2-fluorophenyl)-3′,3′-difluoro-[1,4′-bipiperidine]-1′-carboxylate (200 mg, 381 μmol, 1.00 eq) in DCM (5 mL) was added TFA (3.07 g, 26.9 mmol, 2 mL, 70.6 eq) at 20 °C. The mixture was stirred at 20 °C for 1 h. TLC (PE:THF=0:1, Rf=0.08) indicated tert-butyl 4-(4-((2,6-dioxopiperidin-3-yl)amino)-2-fluorophenyl)-3′,3′-difluoro-[1,4′-bipiperidine]-1′-carboxylate was consumed completely and one main new spot formed. The reaction mixture was concentrated under high vacuum to give the title compound (200 mg, crude, TFA) as a green solid. LCMS (ES⁺): m/z 425.2 [M+H]⁺.

Synthesis of trans-4-((5-fluoro-4-(3-(2-oxopyridin-1 (2H)-yl)phenyl)pyrimidin-2-yl)amino)cyclohexyl 4-(4-((2,6-dioxopiperidin-3-yl)amino)-2-fluorophenyl)-3′,3′-difluoro-[1,4′-bipiperidine]-1′-carboxylate (Compound 648). To a solution of 3-((4-(3′,3′-difluoro-[1,4′-bipiperidin]-4-yl)-3-fluorophenyl)amino)piperidine-2,6-dione (180 mg, 334 μmol, 1.00 eq, TFA) in DMF (3 mL) was added DIEA (86.4 mg, 668 μmol, 116 μL, 2.00 eq) and trans-4-((5-fluoro-4-(3-(2-oxopyridin-1 (2H)-yl)phenyl)pyrimidin-2-yl)amino)cyclohexyl (4-nitrophenyl) carbonate (91.1 mg, 167 μmol, 0.500 eq) at 25 °C. The mixture was stirred at 25 °C for 1 h. LC-MS showed the desired mass was detected. The reaction mixture was filtered and the filtrate concentrated under high vacuum. The residue was purified by prep-HPLC (Waters Xbridge Prep OBD C18 150*40 mm*10 um; mobile phase: [H₂O (10 mM NH₄HCO₃)-ACN]; gradient: 45%-75% B over 8.0 min to give the title compound (Compound 648) (32.7 mg, 39.1 μmol, 11.7% yield, 99.4% purity) as a pale yellow solid. LCMS (ES⁺): m/z 831.4 [M+H]⁺.

### Example 628. Synthesis of trans-4-((5-fluoro-4-(3-(2-oxopyridin-1 (2H)-yl)phenyl)pyrimidin-2-yl)amino)cyclohexyl 4-(1-(4-((2,6-dioxopiperidin-3-yl)amino)-2-fluorophenyl)piperidin-4-yl)piperazine-1-carboxylate (Compound 698)

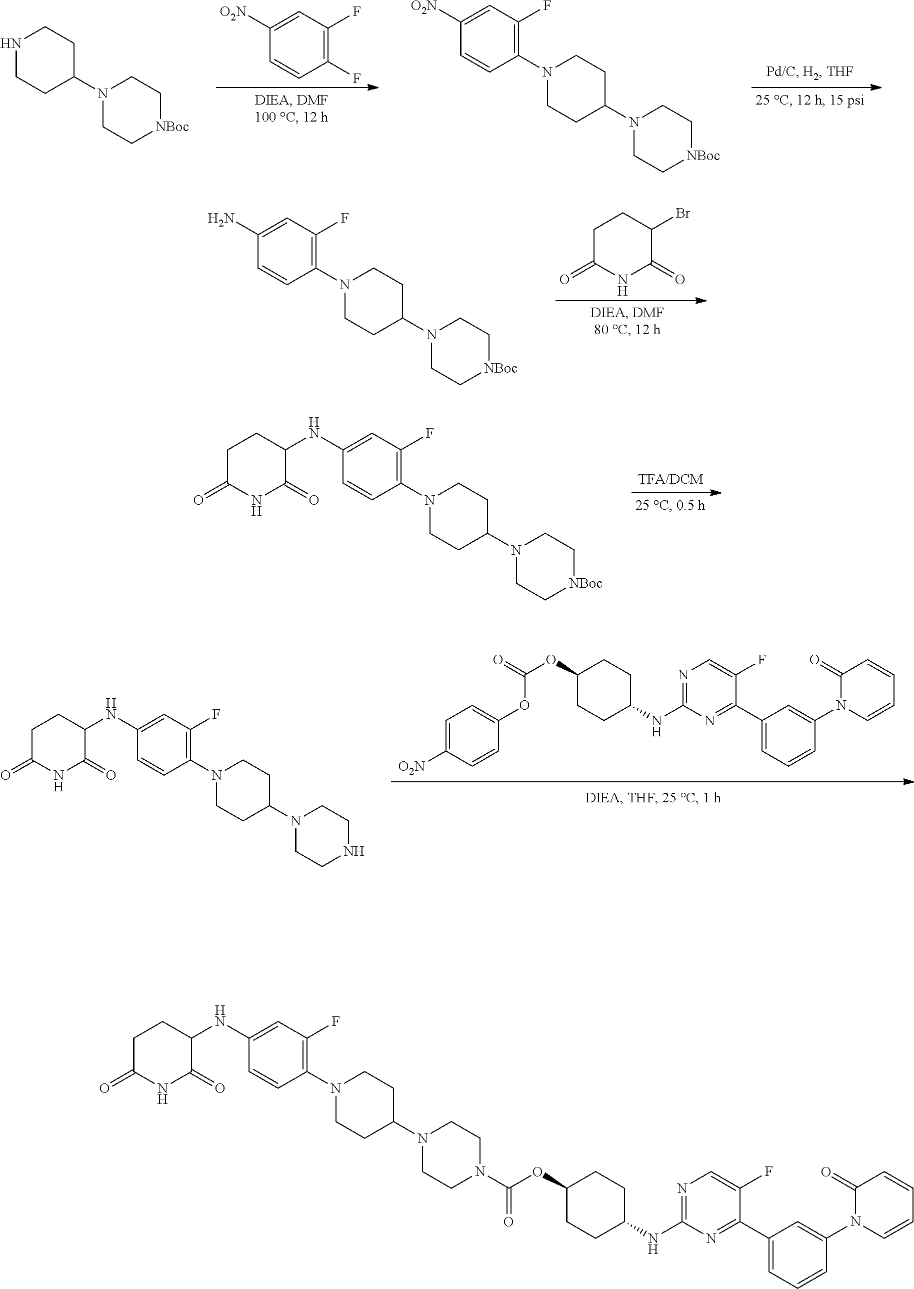

Synthesis of tert-butyl 4-(1-(2-fluoro-4-nitrophenyl)piperidin-4-yl)piperazine-1-carboxylate. To a solution of tert-butyl 4-(piperidin-4-yl)piperazine-1-carboxylate (7.00 g, 25.99 mmol, 1.00 eq) and 1,2-difluoro-4-nitrobenzene (4.13 g, 26.0 mmol, 2.88 mL, 1.00 eq) in DMF (100 mL) was added DIEA (10.1 g, 78.0 mmol, 13.6 mL, 3.00 eq) at 25 °C. The mixture was stirred at 100 °C for 12 h. LCMS showed tert-butyl 4-(piperidin-4-yl)piperazine-1-carboxylate was consumed and the desired target MS was detected. H₂O (100 mL) was added to the mixture and the mixture was filtered. The filter cake was dried under reduced pressure to give the title compound (8.00 g, crude) as a yellow solid. LCMS (ES+): m/z 409.2 [M+H]⁺.

Synthesis of tert-butyl 4-(1-(4-amino-2-fluorophenyl)piperidin-4-yl)piperazine-1-carboxylate. To 10% Pd/C (7.00 g, 50% purity) was added THF (70 mL) at 25 °C under N₂. Then tert-butyl 4-(1-(2-fluoro-4-nitrophenyl)piperidin-4-yl)piperazine-1-carboxylate (7.00 g, 17.1 mmol, 1.00 eq) was added to the mixture at 25 °C and the mixture was degassed and purged with H₂ for three times. The resulting mixture was stirred at 25 °C for 12 h under H₂ (15 psi). LCMS showed tert-butyl 4-(1-(2-fluoro-4-nitrophenyl)piperidin-4-yl)piperazine-1-carboxylate was consumed completely and the desired mass was detected. The mixture was filtered and the filtrate was concentrate under reduced pressure to give the title compound (6.49 g, crude) as a yellow oil. LCMS (ES+): m/z 379.2 [M+H]⁺.

Synthesis of tert-butyl 4-(1-(4-((2,6-dioxopiperidin-3-yl)amino)-2-fluorophenyl)piperidin-4-yl)piperazine-1-carboxylate. To a stirred solution of tert-butyl 4-(1-(4-amino-2-fluorophenyl)piperidin-4-yl)piperazine-1-carboxylate (3.00 g, 7.93 mmol, 1.00 eq) and DIEA (2.05 g, 15.9 mmol, 2.76 mL, 2.00 eq) in DMF (60 mL) was added 3-bromopiperidine-2,6-dione (3.80 g, 19.82 mmol, 2.50 eq) at 25 °C. The resulting mixture was stirred at 80 °C for 12 h. LCMS showed tert-butyl 4-(1-(4-amino-2-fluorophenyl)piperidin-4-yl)piperazine-1-carboxylate was consumed completely and the desired mass was detected. To the mixture was added H₂O (60 mL) and then the mixture was extracted with EtOAc (30 mL*3). The combined organic layers were dried over Na₂SO₄, filtered and the filtrate was concentrated under reduced pressure to give a residue. The residue was purified by MPLC (SiO2, PE/EtOAc=1/0 to 0/1) to give the title compound (1.10 g, 2.25 mmol, 28.4% yield) as a green solid. LCMS (ES+): m/z 490.3 [M+H]⁺.

Synthesis of 3-((3-fluoro-4-(4-(piperazin-1-yl)piperidin-1-yl)phenyl)amino)piperidine-2,6-dione. A solution of tert-butyl 4-(1-(4-((2,6-dioxopiperidin-3-yl)amino)-2-fluorophenyl)piperidin-4-yl)piperazine-1-carboxylate (200 mg, 409 μmol, 1.00 eq) in DCM (3 mL) and TFA (1 mL) was stirred at 25 °C for 0.5 h. LCMS showed tert-butyl 4-(1-(4-((2,6-dioxopiperidin-3-yl)amino)-2-fluorophenyl)piperidin-4-yl)piperazine-1-carboxylate was consumed completely and the desired mass was detected. The mixture was concentrated under reduced pressure to give the title compound (205 mg, crude, TFA) as a yellow oil. LCMS (ES+): m/z 390.1 [M+H]⁺.

Synthesis of trans-4-((5-fluoro-4-(3-(2-oxopyridin-1 (2H)-yl)phenyl)pyrimidin-2-yl)amino)cyclohexyl 4-(1-(4-((2,6-dioxopiperidin-3-yl)amino)-2-fluorophenyl)piperidin-4-yl)piperazine-1-carboxylate (Compound 698). To a stirred solution of 3-((3-fluoro-4-(4-(piperazin-1-yl)piperidin-1-yl)phenyl)amino)piperidine-2,6-dione (205 mg, 407 μmol, 1.00 eq, TFA) and DIEA (340 mg, 2.63 mmol, 458 μL, 5.00 eq) in THF (3 mL) was added trans-(1r,4r)-4-((5-fluoro-4-(3-(2-oxopyridin-1 (2H)-yl)phenyl)pyrimidin-2-yl)amino)cyclohexyl (4-nitrophenyl) carbonate (230 mg, 421 μmol, 0.800 eq) in THE (2 mL) at 25 °C. The resulting mixture was stirred at 25 °C for 1 h. LCMS showed 3-((3-fluoro-4-(4-(piperazin-1-yl)piperidin-1-yl)phenyl)amino)piperidine-2,6-dione was consumed completely and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 40%-70% B over 8.0 min) to give the title compound (Compound 698) (41.2 mg, 47.0 μmol, 8.94% yield, 90.9% purity) as a pale yellow solid. LCMS (ES+): m/z 796.4 [M+H]⁺

### Example 629. Synthesis of trans-4-((5-fluoro-4-(3-(2-oxopyridin-1 (2H)-yl)phenyl)pyrimidin-2-yl)amino)cyclohexyl 4-(2-(4-(4-((2,6-dioxopiperidin-3-yl)amino)-2-fluorophenyl)piperidin-1-yl)ethyl)piperidine-1-carboxylate (Compound 697)

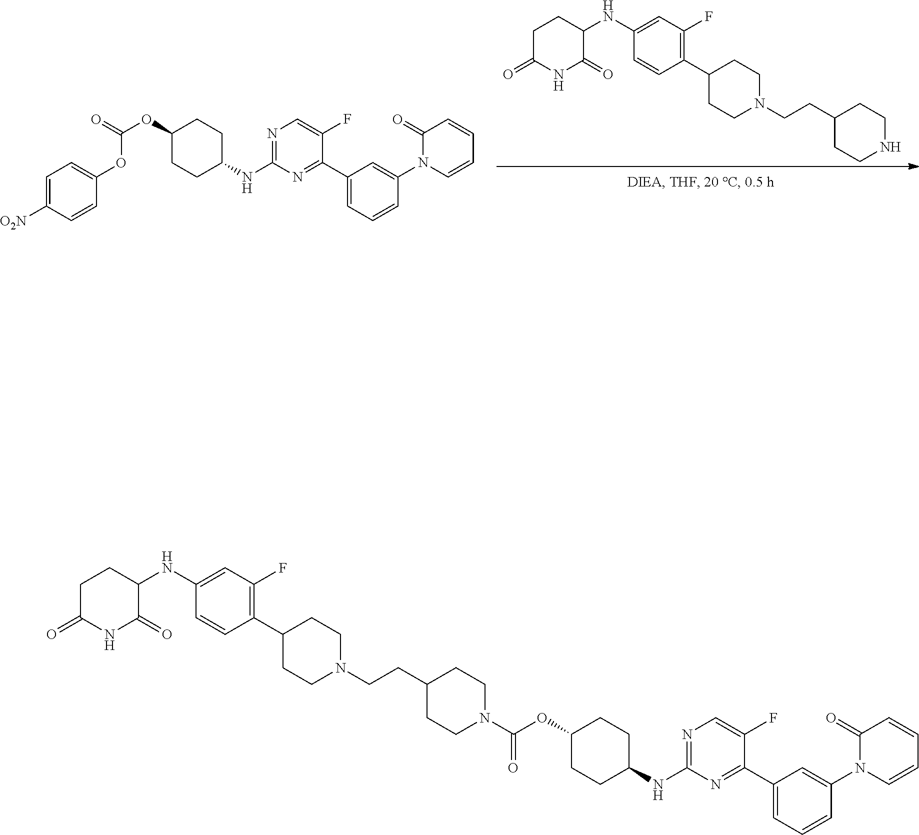 [SMILES: O=C(Oc1ccc([N+](=O)[O-])cc1)O[C@H]1CC[C@H](Nc2ncc(F)c(-c3cccc(-n4ccccc4=O)c3)n2)CC1 ; O=C1CCC(Nc2ccc(C3CCN(CCC4CCNCC4)CC3)c(F)c2)C(=O)N1 ; O=C1CCC(Nc2ccc(C3CCN(CCC4CCN(C(=O)O[C@H]5CC[C@H](Nc6ncc(F)c(-c7cccc(-n8ccccc8=O)c7)n6)CC5)CC4)CC3)c(F)c2)C(=O)N1]

Synthesis of trans-4-((5-fluoro-4-(3-(2-oxopyridin-1 (2H)-yl)phenyl)pyrimidin-2-yl)amino)cyclohexyl 4-(2-(4-(4-((2,6-dioxopiperidin-3-yl)amino)-2-fluorophenyl)piperidin-1-yl)ethyl)piperidine-1-carboxylate (Compound 697). To a solution of 3-((3-fluoro-4-(1-(2-(piperidin-4-yl)ethyl)piperidin-4-yl)phenyl)amino)piperidine-2,6-dione (200 mg, 377 μmol, 1.00 eq, TFA) and trans-4-((5-fluoro-4-(3-(2-oxopyridin-1 (2H)-yl)phenyl)pyrimidin-2-yl)amino)cyclohexyl (4-nitrophenyl) carbonate (103 mg, 188 μmol, 0.500 eq) in THF (6 mL) was added DIEA (244 mg, 1.88 mmol, 328 μL, 5.00 eq) at 20 °C. The mixture was stirred at 20 °C for 0.5 h. LCMS showed 3-((3-fluoro-4-(1-(2-(piperidin-4-yl)ethyl)piperidin-4-yl)phenyl)amino)piperidine-2,6-dione was consumed and the desired target MS was detected. The mixture was concentrated under reduced pressure to give a residue. The residue was purified by prep-HPLC (column: Phenomenex luna C18 100*40 mm*3 um; mobile phase: [H₂O (0.2% FA)-ACN]; gradient: 15%-50% B over 8.0 min) to give the title compound (Compound 697) (31.2 mg, 37.5 μmol, 9.94% yield, 98.9% purity) as a pale yellow solid. LCMS (ES+): m/z 823.4 [M+H]⁺/412.3[M/2+H]⁺

### Example 630. Synthesis of (trans)-4-((5-fluoro-4-(3-(2-oxopyridin-1 (2H)-yl)phenyl)pyrimidin-2-yl)amino)cyclohexyl 6-(4-(4-((2,6-dioxopiperidin-3-yl)amino)-2-fluorophenyl)piperidin-1-yl)-2-azaspiro[3.3]heptane-2-carboxylate (Compound 666)

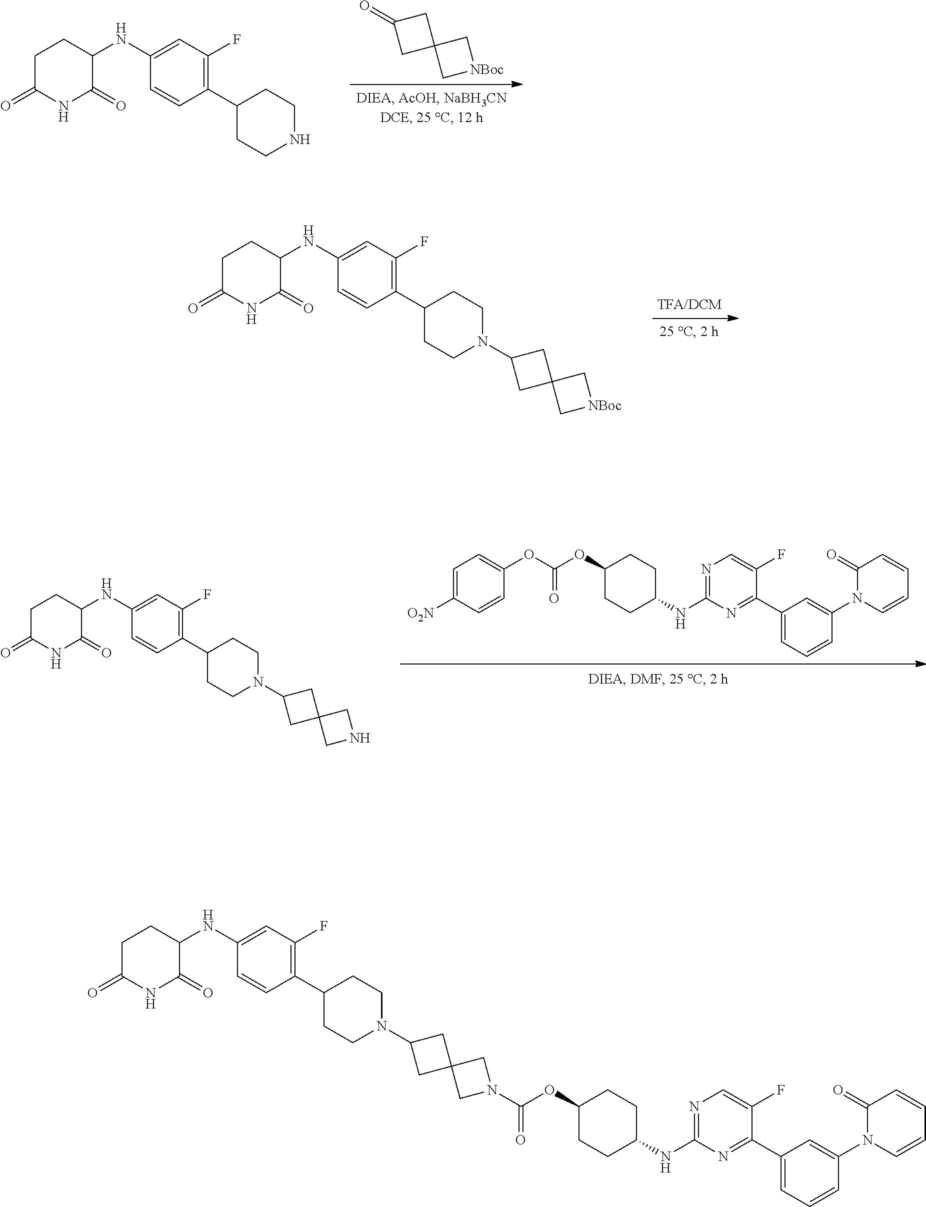 [SMILES: O=C1CCC(Nc2ccc(C3CCNCC3)c(F)c2)C(=O)N1 ; CC(C)(C)OC(=O)N1CC2(CC(=O)C2)C1 ; CC(C)(C)OC(=O)N1CC2(CC(N3CCC(c4ccc(NC5CCC(=O)NC5=O)cc4F)CC3)C2)C1 ; O=C1CCC(Nc2ccc(C3CCN(C4CC5(CNC5)C4)CC3)c(F)c2)C(=O)N1 ; O=C(Oc1ccc([N+](=O)[O-])cc1)O[C@H]1CC[C@H](Nc2ncc(F)c(-c3cccc(-n4ccccc4=O)c3)n2)CC1 ; O=C1CCC(Nc2ccc(C3CCN(C4CC5(C4)CN(C(=O)O[C@H]4CC[C@H](Nc6ncc(F)c(-c7cccc(-n8ccccc8=O)c7)n6)CC4)C5)CC3)c(F)c2)C(=O)N1]

Synthesis of tert-butyl 6-(4-(4-((2,6-dioxopiperidin-3-yl)amino)-2-fluorophenyl)piperidin-1-yl)-2-azaspiro[3.3]heptane-2-carboxylate. To a solution of 3-((3-fluoro-4-(piperidin-4-yl)phenyl)amino)piperidine-2,6-dione (1.00 g, 3.27 mmol, 1.00 eq) in DCE (30 mL) was added dropwise AcOH (196 mg, 3.27 mmol, 187 μL, 1.00 eq) and tert-butyl 6-oxo-2-azaspiro[3.3]heptane-2-carboxylate (1.38 g, 6.55 mmol, 2.00 eq) at 25 °C over 0.5 h. After addition, the mixture was stirred at this temperature for 0.5 h, and then NaBH₃CN (205 mg, 3.27 mmol, 1.00 eq) was added to the mixture at 25 °C. The resulting mixture was stirred at 25 °C for 11 h. LC-MS showed the desired mass was detected. The reaction mixture was quenched by addition H₂O 10 mL, and then diluted with H₂O 10 mL and extracted with EtOAc (10 mL*3). The combined organic layers were washed with sat.aq. NaCl (10 mL), dried over Na₂SO₄, filtered and concentrated under reduced pressure. The residue was purified by column chromatography (SiO₂, PE:THF=1:0 to 0:1) to give the title compound (1.20 g, 1.49 mmol, 45.5% yield, 62.2% purity) as a colourless oil. LCMS (ES⁺): m/z 501.3 [M+H]⁺.

Synthesis of 3-((4-(1-(2-azaspiro[3.3]heptan-6-yl)piperidin-4-yl)-3-fluorophenyl)amino)piperidine-2,6-dione. To a solution of tert-butyl 6-(4-(4-((2,6-dioxopiperidin-3-yl)amino)-2-fluorophenyl)piperidin-1-yl)-2-azaspiro[3.3]heptane-2-carboxylate (200 mg, 399 μmol, 1.00 eq) in DCM (2 mL) was added TFA (1.84 g, 16.1 mmol, 1.20 mL, 40.4 eq) at 25 °C. The mixture was stirred at 25 °C for 2 h. TLC (PE:THF=0:1, Rf=0.21) indicated tert-butyl 6-(4-(4-((2,6-dioxopiperidin-3-yl)amino)-2-fluorophenyl)piperidin-1-yl)-2-azaspiro[3.3]heptane-2-carboxylate was consumed completely and one main new spot formed. The reaction mixture was concentrated under high vacuum to give the title compound (200 mg, crude, TFA) as a yellow solid. LCMS (ES⁺): m/z 401.2 [M+H]⁺.

Synthesis of trans-4-((5-fluoro-4-(3-(2-oxopyridin-1 (2H)-yl)phenyl)pyrimidin-2-yl)amino)cyclohexyl 6-(4-(4-((2,6-dioxopiperidin-3-yl)amino)-2-fluorophenyl)piperidin-1-yl)-2-azaspiro[3.3]heptane-2-carboxylate (Compound 666). To a solution of 3-((4-(1-(2-azaspiro[3.3]heptan-6-yl)piperidin-4-yl)-3-fluorophenyl)amino)piperidine-2,6-dione (200 mg, 388 μmol, 1.00 eq, TFA) in DMF (4 mL) was added DIEA (50.2 mg, 388 μmol, 67.7 μL, 1.00 eq) and trans-4-((5-fluoro-4-(3-(2-oxopyridin-1 (2H)-yl)phenyl)pyrimidin-2-yl)amino)cyclohexyl (4-nitrophenyl) carbonate (212 mg, 388 μmol, 1.00 eq) at 25 °C. The mixture was stirred at 25 °C for 2 h. LC-MS showed the desired mass was detected. The reaction mixture was concentrated under high vacuum. The residue was purified by prep-HPLC (Phenomenex luna C18 100*40 mm*5 um; mobile phase: [H₂O (0.2% FA)-ACN]; gradient: 10%-50% B over 8.0 min) to give the title compound (Compound 666) (123 mg, 148 μmol, 38.1% yield, 100% purity, 0.5 FA) as a pale yellow solid. LCMS (ES⁺): m/z 807.4 [M+H]⁺.

### Example 631. Synthesis of trans-4-((5-fluoro-4-(3-(2-oxopyridin-1 (2H)-yl)phenyl)pyrimidin-2-yl)amino)cyclohexyl 6-(4-(4-((2,6-dioxopiperidin-3-yl)amino)-2-fluorophenyl)piperazin-1-yl)-2-azaspiro[3.3]heptane-2-carboxylate (Compound 681)

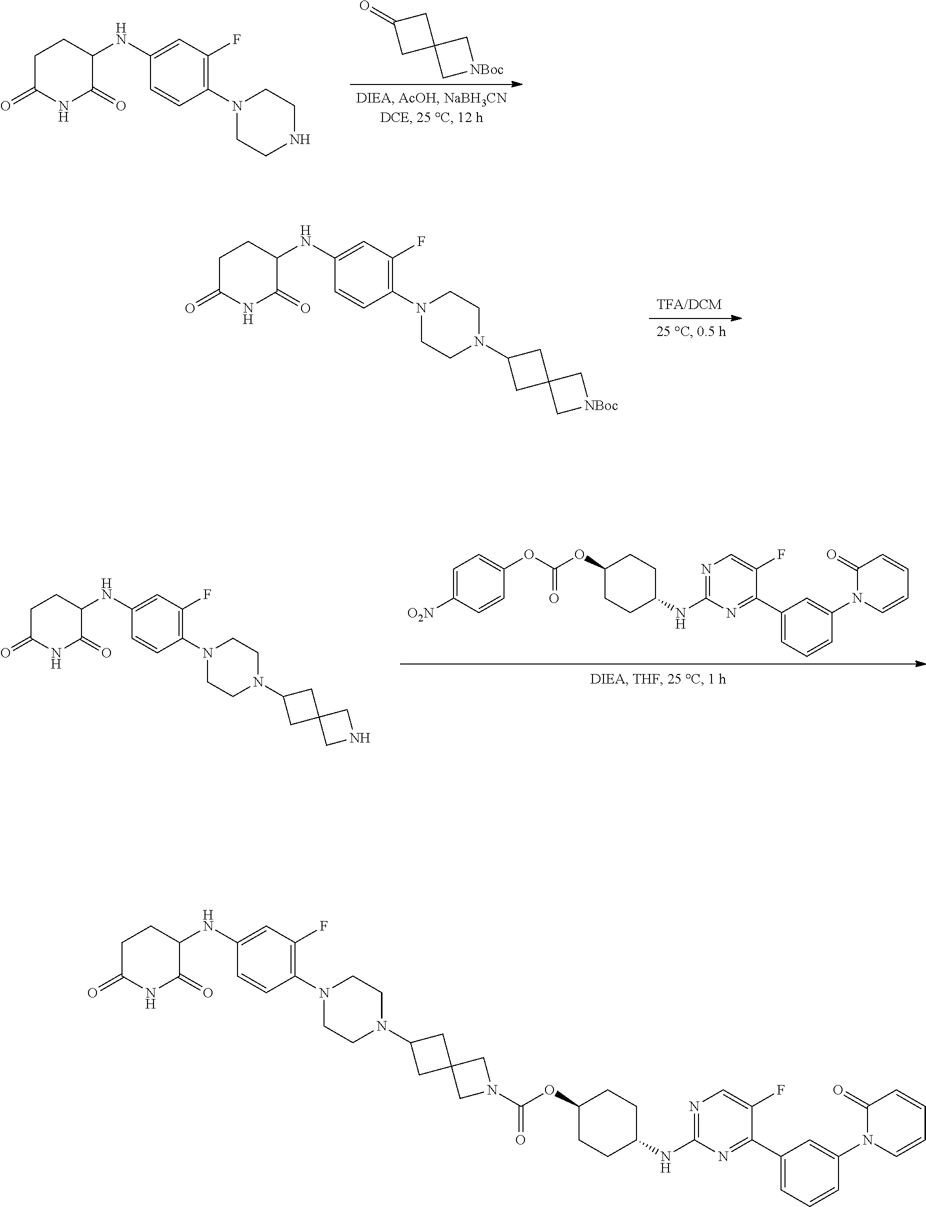 [SMILES: O=C1CCC(Nc2ccc(N3CCNCC3)c(F)c2)C(=O)N1 ; CC(C)(C)OC(=O)N1CC2(CC(=O)C2)C1 ; CC(C)(C)OC(=O)N1CC2(CC(N3CCN(c4ccc(NC5CCC(=O)NC5=O)cc4F)CC3)C2)C1 ; O=C1CCC(Nc2ccc(N3CCN(C4CC5(CNC5)C4)CC3)c(F)c2)C(=O)N1 ; O=C(Oc1ccc([N+](=O)[O-])cc1)O[C@H]1CC[C@H](Nc2ncc(F)c(-c3cccc(-n4ccccc4=O)c3)n2)CC1 ; O=C1CCC(Nc2ccc(N3CCN(C4CC5(C4)CN(C(=O)O[C@H]4CC[C@H](Nc6ncc(F)c(-c7cccc(-n8ccccc8=O)c7)n6)CC4)C5)CC3)c(F)c2)C(=O)N1]

Synthesis of tert-butyl 6-(4-(4-((2,6-dioxopiperidin-3-yl)amino)-2-fluorophenyl)piperazin-1-yl)-2-azaspiro[3.3]heptane-2-carboxylate. To a solution of 3-(3-fluoro-4-piperazin-1-yl-anilino)piperidine-2,6-dione (500 mg, 1.19 mmol, 1.00 eq, TFA) in DCM (10 mL) was added dropwise DIEA (461 mg, 3.57 mmol, 621 μL, 3.00 eq), and tert-butyl 6-oxo-2-azaspiro[3.3]heptane-2-carboxylate (502 mg, 2.38 mmol, 2.00 eq) at 25 °C over 1 h. After addition, the mixture was stirred 25 °C for 1 h, then AcOH (214 mg, 3.57 mmol, 204 μL, 3.00 eq) was added to the mixture at 25 °C, then the mixture was stirred at 25 for 1 h. Then NaBH₃CN (224 mg, 3.57 mmol, 3.00 eq) was added to the mixture at 25 °C. The resulting mixture was stirred at 25 °C for 10 h. LC-MS showed the desired mass was detected. The reaction mixture was quenched by addition H₂O 10 mL, and then diluted with H₂O 30 mL and extracted with EtOAc 30 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (300 mg, crude) as a black oil. LCMS (ES+): m/z 502.2 [M+H]⁺.

Synthesis of 3-((4-(4-(2-azaspiro[3.3]heptan-6-yl)piperazin-1-yl)-3-fluorophenyl)amino)piperidine-2,6-dione. To a solution of tert-butyl 6-[4-[4-[(2,6-dioxo-3-piperidyl)amino]-2-fluoro-phenyl]piperazin-1-yl]-2-azaspiro[3.3]heptane-2-carboxylate (200 mg, 398 μmol, 1.00 eq) in DCM (3 mL) was added TFA (3.07 g, 26.9 mmol, 2.00 mL, 67.5 eq) at 25 °C. The mixture was stirred at 25 °C for 0.5 h. LC-MS showed desired mass was detected. The reaction mixture was concentrated under reduced pressure to give the title compound (200 mg, crude, TFA) as a black oil. LCMS (ES+): m/z 402.2 [M+H]⁺.

Synthesis of trans-4-((5-fluoro-4-(3-(2-oxopyridin-1 (2H)-yl)phenyl)pyrimidin-2-yl)amino)cyclohexyl 6-(4-(4-((2,6-dioxopiperidin-3-yl)amino)-2-fluorophenyl)piperazin-1-yl)-2-azaspiro[3.3]heptane-2-carboxylate (Compound 681). To a solution of 3-[4-[4-(2-azaspiro[3.3]heptan-6-yl)piperazin-1-yl]-3-fluoro-anilino]piperidine-2,6-dione (200 mg, 387 mol, 1.00 eq, TFA) and trans-4-((5-fluoro-4-(3-(2-oxopyridin-1 (2H)-yl)phenyl)pyrimidin-2-yl)amino)cyclohexyl (4-nitrophenyl) carbonate (211 mg, 387 μmol, 1.00 eq) in THF (4 mL) was added DIEA (100 mg, 775 μmol, 135 μL, 2.00 eq) at 25 °C. The mixture was stirred at 25 °C for 1 h. LC-MS showed the desired mass was detected. The reaction mixture was concentrated under reduced pressure. The residue was purified by prep-HPLC (column: Waters Xbridge BEH C18 100*30 mm*10 um; mobile phase: [H₂O (10 mM NH₄HCO₃)-ACN]; gradient: 28%-58% B over 8.0 min) to give the title compound (Compound 681) (81.1 mg, 98.4 μmol, 25.3% yield, 98.1% purity) as a white solid. LCMS (ES+): m/z 808.4 [M+H]+.

### Example 632. Synthesis of (trans)-4-((5-fluoro-4-(3-(2-oxopyridin-1 (2H)-yl)phenyl)pyrimidin-2-yl)amino)cyclohexyl 6-(1-(4-((2,6-dioxopiperidin-3-yl)amino)-2-fluorophenyl)piperidin-4-yl)-2,6-diazaspiro[3.3]heptane-2-carboxylate (Compound 652)

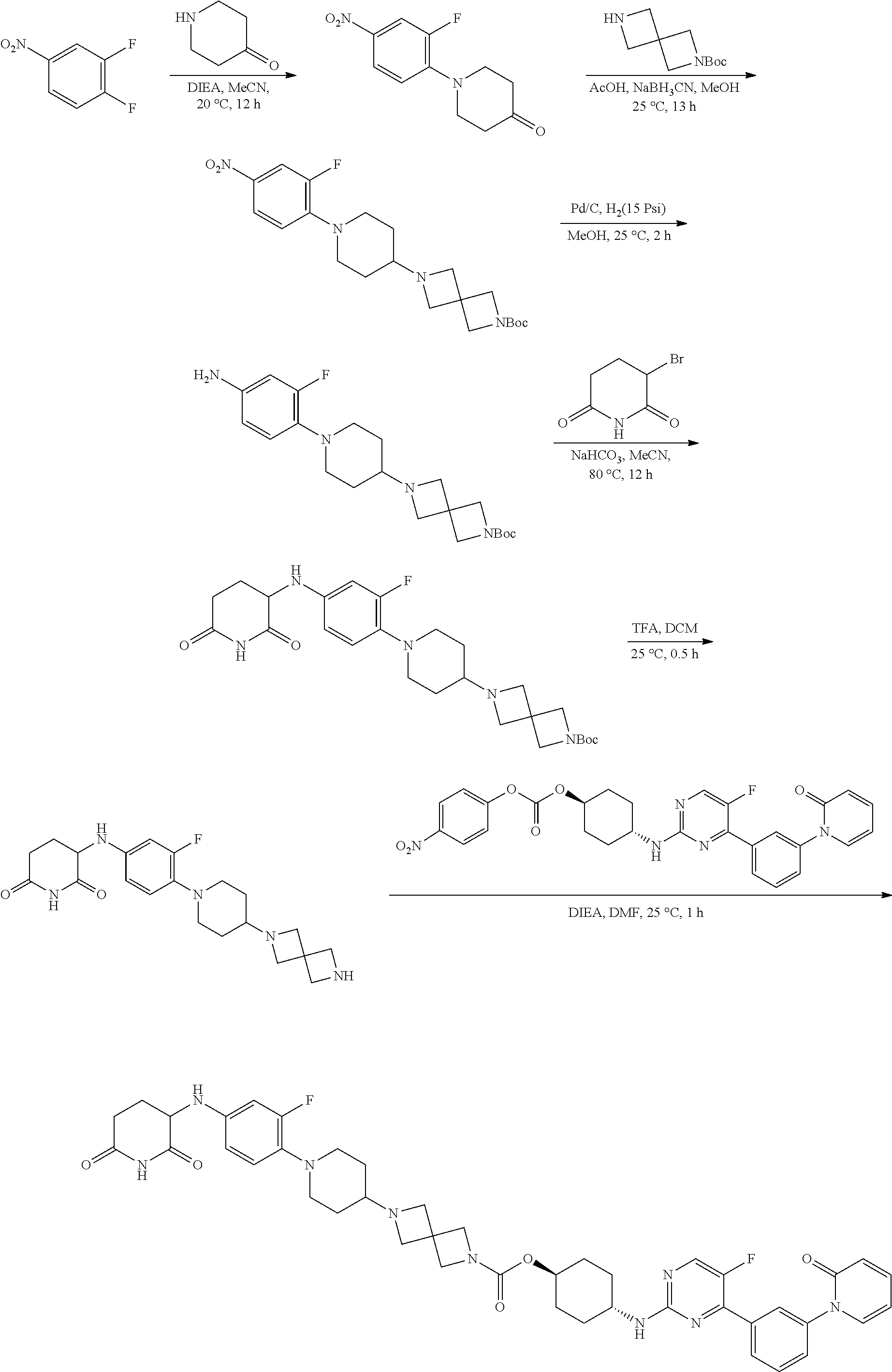

Synthesis of 1-(2-fluoro-4-nitro-phenyl)piperidin-4-one. The reaction was set up in two batches. To a solution of 1,2-difluoro-4-nitro-benzene (25.0 g, 157 mmol, 17.4 mL, 1.00 eq) in MeCN (50 mL) was added DIEA (60.9 g, 471 mmol, 82.1 mL, 3.00 eq) and piperidin-4-one (15.5 g, 157 mmol, 1.00 eq) at 25 °C. The mixture was stirred at 80 °C for 12 h. LC-MS showed the desired mass was detected. The reaction mixture was concentrated under reduced pressure. The crude product was purified by re-crystallization from PE (300 mL) at 25 °C for 0.5 h to give the title compound (50.0 g, 207 mmol, 66.1% yield, 99.0% purity) as a brown solid. LCMS (ES⁺): m/z 239.1[M+H]⁺.

Synthesis of tert-butyl 6-[1-(2-fluoro-4-nitro-phenyl)-4-piperidyl]-2,6-diazaspiro[3.3]heptane-2-carboxylate. To a solution of tert-butyl 1-(2-fluoro-4-nitro-phenyl)piperidin-4-one (3.00 g, 12.5 mmol, 1.00 eq) in MeOH (50 mL) was added, AcOH (756 mg, 12.5 mmol, 720 μL, 1.00 eq), tert-butyl 2,6-diazaspiro[3.3]heptane-2-carboxylate (2.50 g, 12.5 mmol, 1.00 eq) at 25 °C. After addition, the mixture was stirred at this temperature for 1 h, and then NaBH₃CN (1.58 g, 25.1 mmol, 2.00 eq) was added to the mixture at 25 °C. The resulting mixture was stirred at 25 °C for 12 h. LC-MS showed the desired mass was detected. The reaction mixture was quenched by ice water 100 mL at 0 °C, and then extracted with EtOAc (100 mL*3). The combined organic layers were washed with brine (50 mL), dried over Na₂SO₄, filtered and concentrated under reduced pressure. The residue was purified by column chromatography (SiO₂, Petroleum ether:Ethyl acetate=1:0 to 3:1) to give the title compound (5.00 g, 11.9 mmol, 94.4% yield) as a yellow solid. LCMS (ES⁺): m/z 421.2 [M+H]⁺.

Synthesis of tert-butyl 6-[1-(4-amino-2-fluoro-phenyl)-4-piperidyl]-2,6-diazaspiro[3.3]heptane-2-carboxylate. A mixture of tert-butyl 6-[1-(2-fluoro-4-nitro-phenyl)-4-piperidyl]-2,6-diazaspiro[3.3]heptane-2-carboxylate (5.00 g, 11.8 mmol, 1.00 eq), 10% Pd/C (3.33 g, 15.6 mmol, 50.0% purity, 1.32 eq) in MeOH (50 mL) was degassed and purged with N₂ for 3 times, and then the mixture was stirred at 25 °C for 2 h under H₂ (15 psi) atmosphere. LC-MS showed the desired mass was detected. The reaction mixture was concentrated under reduced pressure to give the title compound (4.00 g, crude) as a gray solid. LCMS (ES⁺): m/z 391.2 [M+H]⁺.

Synthesis of tert-butyl 6-[1-[4-[(2,6-dioxo-3-piperidyl)amino]-2-fluoro-phenyl]-4-piperidyl]-2,6-diazaspiro[3.3]heptane-2-carboxylate. To a solution of tert-butyl 6-[1-(4-amino-2-fluoro-phenyl)-4-piperidyl]-2,6-diazaspiro[3.3]heptane-2-carboxylate (1.00 g, 2.56 mmol, 1.00 eq) in MeCN (50 mL) was added NaHCO₃ (430 mg, 5.12 mmol, 199 μL, 2.00 eq) and 3-bromopiperidine-2,6-dione (1.48 g, 7.68 mmol, 3.00 eq) at 25 °C. The mixture was stirred at 80 °C for 12 h. LC-MS showed the desired mass was detected. The reaction mixture was quenched by ice water 20 mL at 0 °C, and then extracted with EtOAc (20 mL*3). The combined organic layers were washed with brine (20 mL), dried over Na₂SO₄, filtered and concentrated under reduced pressure The residue was purified by column chromatography (SiO₂, Petroleum ether:Ethyl acetate=1:0 to 3:1) to give the title compound (300 mg, 598 μmol, 23.3% yield) as a yellow oil. LCMS (ES⁺): m/z 502.3 [M+H]⁺.

Synthesis of 3-[4-[4-(2,6-diazaspiro[3.3]heptan-2-yl)-1-piperidyl]-3-fluoro-anilino]piperidine-2,6-dione. To a solution of tert-butyl 6-[1-[4-[(2,6-dioxo-3-piperidyl)amino]-2-fluoro-phenyl]-4-piperidyl]-2,6-diazaspiro[3.3]heptane-2-carboxylate (300 mg, 598 μmol, 1.00 eq) in DCM (1 mL) was added TFA (767 mg, 6.73 mmol, 0.50 mL, 11.2 eq). The mixture was stirred at 25 °C for 0.5 h. LC-MS showed the desired mass was detected. The reaction mixture was concentrated under reduced pressure to give the title compound (200 mg, crude, TFA) as a yellow oil. LCMS (ES⁺): m/z 402.3[M+H]⁺.

Synthesis of (trans)-4-((5-fluoro-4-(3-(2-oxopyridin-1 (2H)-yl)phenyl)pyrimidin-2-yl)amino)cyclohexyl 6-(1-(4-((2,6-dioxopiperidin-3-yl)amino)-2-fluorophenyl)piperidin-4-yl)-2,6-diazaspiro[3.3]heptane-2-carboxylate (Compound 652). To a solution of 3-[4-[4-(2,6-diazaspiro[3.3]heptan-2-yl)-1-piperidyl]-3-fluoro-anilino]piperidine-2,6-dione (150 mg, 290 μmol, 1.00 eq, TFA) in DMF (2 mL) was added DIEA (75.2 mg, 581 μmol, 101 μL, 2.00 eq) and [4-[[5-fluoro-4-[3-(2-oxo-1-pyridyl)phenyl]pyrimidin-2-yl]amino]cyclohexyl](4-nitrophenyl) carbonate (158 mg, 290 μmol, 1.00 eq) at 25 °C. The mixture was stirred at 25 °C for 1 h. LC-MS showed the desired mass was detected. The reaction mixture was quenched by addition H₂O 3 mL, and then extracted with EtOAc (3 mL*3). The combined organic layers were dried over Na₂SO₄, filtered and concentrated under reduced pressure. The residue was purified by column chromatography (SiO₂, Petroleum ether:Ethyl acetate=1:0 to 5:1) to give the title compound (Compound 652) (71.2 mg, 85.5 μmol, 29.4% yield, 99.3% purity, 0.400 FA) as a yellow solid. LCMS (ES⁺): m/z 808.3 [M+H]⁺.

### Example 633. Synthesis of trans-4-((5-fluoro-4-(3-(2-oxopyridin-1 (2H)-yl)phenyl)pyrimidin-2-yl)amino)cyclohexyl 4-(2-(4-(4-((2,6-dioxopiperidin-3-yl)amino)-2-fluorophenyl)piperidin-1-yl)ethyl)-4-hydroxypiperidine-1-carboxylate (Compound 641)

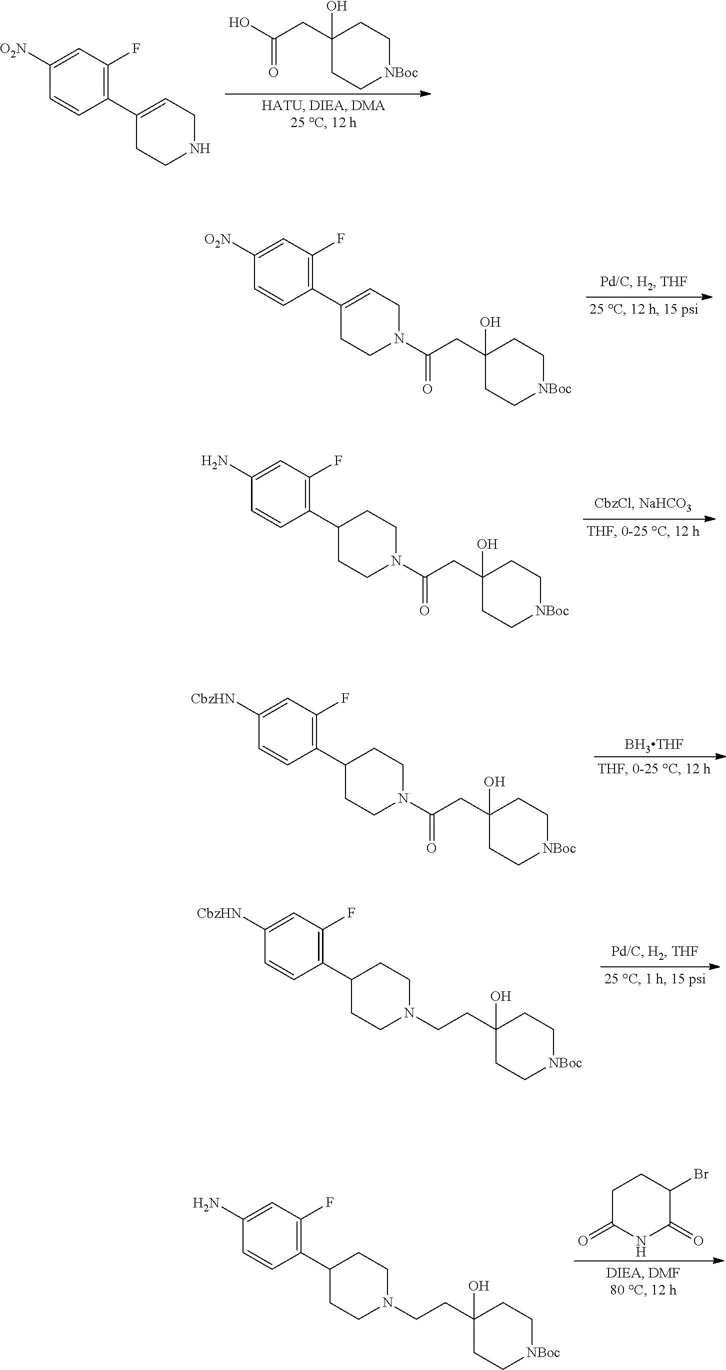

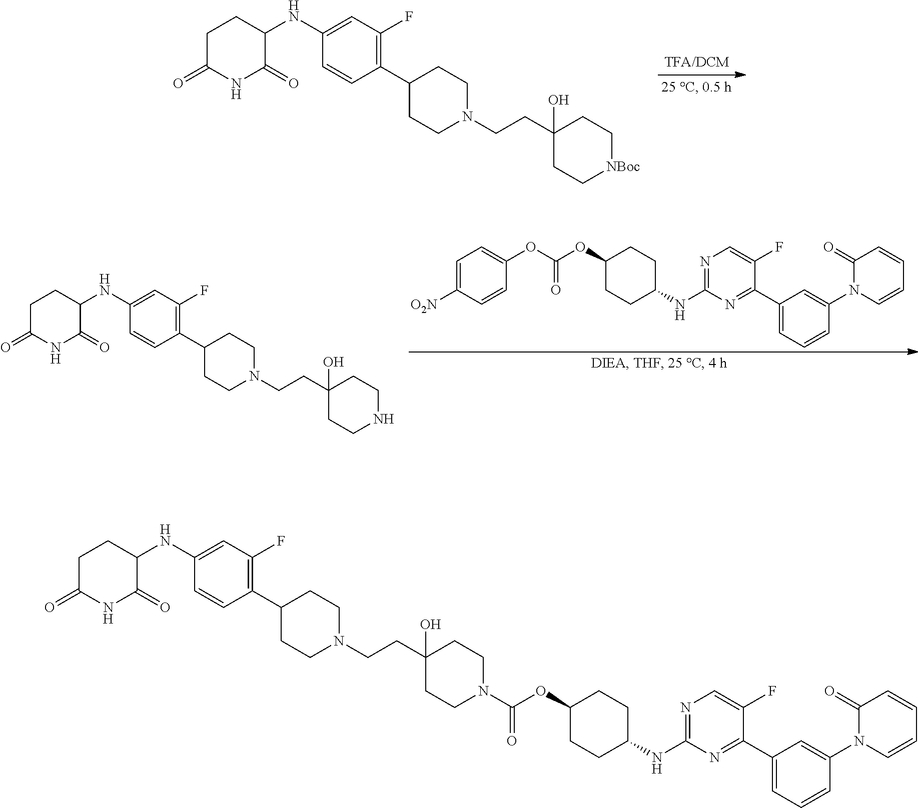 [SMILES: CC(C)(C)OC(=O)N1CCC(O)(CCN2CCC(c3ccc(NC4CCC(=O)NC4=O)cc3F)CC2)CC1 ; O=C1CCC(Nc2ccc(C3CCN(CCC4(O)CCNCC4)CC3)c(F)c2)C(=O)N1 ; O=C(Oc1ccc([N+](=O)[O-])cc1)O[C@H]1CC[C@H](Nc2ncc(F)c(-c3cccc(-n4ccccc4=O)c3)n2)CC1 ; O=C1CCC(Nc2ccc(C3CCN(CCC4(O)CCN(C(=O)O[C@H]5CC[C@H](Nc6ncc(F)c(-c7cccc(-n8ccccc8=O)c7)n6)CC5)CC4)CC3)c(F)c2)C(=O)N1]

Synthesis of tert-butyl 4-(2-(4-(2-fluoro-4-nitrophenyl)-3,6-dihydropyridin-1 (2H)-yl)-2-oxoethyl)-4-hydroxypiperidine-1-carboxylate. To a solution of 2-(1-(tert-butoxycarbonyl)-4-hydroxypiperidin-4-yl)acetic acid (5.40 g, 20.8 mmol, 1.00 eq) in DMF (200 mL) were added HATU (11.9 g, 31.2 mmol, 1.50 eq) and DIEA (13.4 g, 104 mmol, 18.1 mL, 5.00 eq) at 25 °C, then to the mixture was added 4-(2-fluoro-4-nitrophenyl)-1,2,3,6-tetrahydropyridine (7.00 g, 20.8 mmol, 1.00 eq, TFA) at 25 °C, the mixture was stirred at 25 °C for 12 h. LCMS showed 2-(1-(tert-butoxycarbonyl)-4-hydroxypiperidin-4-yl)acetic acid was consumed completely and the desired mass was detected. The mixture was filtered and the filter cake was dried under reduced pressure to give the title compound (8.50 g, crude) as a white solid. LCMS (ES⁺): m/z 408.2 [M+H−56]⁺.

Synthesis of tert-butyl 4-(2-(4-(4-amino-2-fluorophenyl)piperidin-1-yl)-2-oxoethyl)-4-hydroxypiperidine-1-carboxylate. To 10% Pd/C (5.50 g, 50% purity) was added THF (100 mL) at 25 °C under N₂. Then tert-butyl 4-(2-(4-(2-fluoro-4-nitrophenyl)-3,6-dihydropyridin-1 (2H)-yl)-2-oxoethyl)-4-hydroxypiperidine-1-carboxylate (5.00 g, 10.8 mmol, 1.00 eq) was added to the mixture at 25 °C under N₂ and the mixture was degassed and purged with H₂ for three times. The resulting mixture was stirred at 25 °C for 12 h under H₂ (15 psi). LCMS showed 4-(2-(4-(2-fluoro-4-nitrophenyl)-3,6-dihydropyridin-1 (2H)-yl)-2-oxoethyl)-4-hydroxypiperidine-1-carboxylate was consumed completely and the desired mass was detected. The mixture was filtered and the filtrate was concentrate under reduced pressure to give the title compound (6.00 g, crude) as a yellow oil. LCMS (ES⁺): m/z 436.4 [M+H]⁺.

Synthesis of tert-butyl 4-(2-(4-(4-(((benzyloxy)carbonyl)amino)-2-fluorophenyl)piperidin-1-yl)-2-oxoethyl)-4-hydroxypiperidine-1-carboxylate. To a solution of tert-butyl 4-(2-(4-(4-amino-2-fluorophenyl)piperidin-1-yl)-2-oxoethyl)-4-hydroxypiperidine-1-carboxylate (5.80 g, 13.3 mmol, 1.00 eq) in THE (80 mL) were added NaHCO₃ (2.24 g, 26.6 mmol, 1.04 mL, 2.00 eq) and CbzCl (2.50 g, 14.7 mmol, 2.09 mL, 1.10 eq) at 0 °C, the mixture was stirred at 25 °C for 12 h. LCMS showed tert-butyl 4-(2-(4-(4-amino-2-fluorophenyl)piperidin-1-yl)-2-oxoethyl)-4-hydroxypiperidine-1-carboxylate was consumed completely and the desired mass was detected. To the mixture was added H₂O (80 mL) and the mixture was extracted with EtOAc (40 mL*3). The combined organic layers were dried over Na₂SO₄, filtered and the filtrate was concentrated under reduced pressure to give a residue. The residue was purified by MPLC (SiO₂, PE/EtOAc=1/0 to 3/1) to give the title compound (5.00 g, 8.78 mmol, 65.9% yield) as a white solid. LCMS (ES⁺): m/z 514.4 [M+H−56]⁺.

Synthesis of tert-butyl 4-(2-(4-(4-(((benzyloxy)carbonyl)amino)-2-fluorophenyl)piperidin-1-yl)ethyl)-4-hydroxypiperidine-1-carboxylate. To a solution of tert-butyl 4-(2-(4-(4-(((benzyloxy)carbonyl)amino)-2-fluorophenyl)piperidin-1-yl)-2-oxoethyl)-4-hydroxypiperidine-1-carboxylate (1.00 g, 1.76 mmol, 1.00 eq) in THE (15 mL) was added BH₃·THF (1 M, 3.51 mL, 2.00 eq) at 0 °C under N₂, the mixture was stirred at 25 °C under N₂ for 2 h. LCMS showed tert-butyl 4-(2-(4-(4-(((benzyloxy)carbonyl)amino)-2-fluorophenyl)piperidin-1-yl)-2-oxoethyl)-4-hydroxypiperidine-1-carboxylate was consumed and the desired mass was detected. The mixture was quenched by dropping methanol (30 ml) until no air bubbles were formed. The mixture was concentrated under reduced pressure to give a residue. The residue was diluted with H₂O (10 mL) and then extracted with EtOAc (10 mL*3). The combined organic layers were dried over Na₂SO₄, filtered and the filtrate was concentrated under reduced pressure to give a residue. The residue was purified by MPLC (SiO2, PE/EtOAc=1/0 to 3/1) to give title compound (450 mg, 810 μmol, 46.1% yield) as a white solid. LCMS (ES⁺): m/z 556.4 [M+H]⁺.

Synthesis of tert-butyl 4-(2-(4-(4-amino-2-fluorophenyl)piperidin-1-yl)ethyl)-4-hydroxypiperidine-1-carboxylate. To 10% Pd/C (250 mg, 50% purity) was added THE (10 mL) at 25 °C under N₂. Then tert-butyl 4-(2-(4-(4-(((benzyloxy)carbonyl)amino)-2-fluorophenyl)piperidin-1-yl)ethyl)-4-hydroxypiperidine-1-carboxylate (200 mg, 360 μmol, 1.00 eq) was added to the mixture at 25 °C under N₂ and the mixture was degassed and purged with H₂ for three times. The resulting mixture was stirred at 25 °C for 1 h under H₂ (15 psi). LCMS showed tert-butyl 4-(2-(4-(4-(((benzyloxy)carbonyl)amino)-2-fluorophenyl)piperidin-1-yl)ethyl)-4-hydroxypiperidine-1-carboxylate was consumed completely and the desired mass was detected. The mixture was filtered, the filtrate was concentrate under reduced pressure to give the title compound (230 mg, crude) as a yellow oil. LCMS (ES⁺): m/z 422.3 [M+H]⁺.

Synthesis of tert-butyl 4-(2-(4-(4-((2,6-dioxopiperidin-3-yl)amino)-2-fluorophenyl)piperidin-1-yl)ethyl)-4-hydroxypiperidine-1-carboxylate. To a solution of tert-butyl 4-(2-(4-(4-amino-2-fluorophenyl)piperidin-1-yl)ethyl)-4-hydroxypiperidine-1-carboxylate (230 mg, 546 μmol, 1.00 eq) in DMF (5 mL) were added DIEA (141 mg, 1.09 mmol, 190 μL, 2.00 eq) and 3-bromopiperidine-2,6-dione (314 mg, 1.64 mmol, 3.00 eq) at 25 °C, the mixture was stirred at 80 °C for 12 h. LCMS showed tert-butyl 4-(2-(4-(4-amino-2-fluorophenyl)piperidin-1-yl)ethyl)-4-hydroxypiperidine-1-carboxylate was consumed completely and the desired mass was detected. H₂O (10 mL) was added to the mixture and the mixture was extracted with EtOAc (10 mL*3). The combined organic layers were washed with brine (20 mL*3), dried over Na₂SO₄, filtered and the filtrate was concentrated under reduced pressure to give a residue. The residue was purified by prep-TLC (SiO₂, EtOAc/MeOH=10/1) to give the title compound (60.0 mg, 113 μmol, 20.7% yield) as a yellow solid. LCMS (ES⁺): m/z 533.4 [M+H]⁺.

Synthesis of 3-((3-fluoro-4-(1-(2-(4-hydroxypiperidin-4-yl)ethyl)piperidin-4-yl)phenyl)amino)piperidine-2,6-dione. A solution of tert-butyl 4-(2-(4-(4-((2,6-dioxopiperidin-3-yl)amino)-2-fluorophenyl)piperidin-1-yl)ethyl)-4-hydroxypiperidine-1-carboxylate (60.0 mg, 113 μmol, 1.00 eq) in DCM (2.1 mL) and TFA (0.7 mL) was stirred at 25 °C for 0.5 h. LCMS showed tert-butyl 4-(2-(4-(4-((2,6-dioxopiperidin-3-yl)amino)-2-fluorophenyl)piperidin-1-yl)ethyl)-4-hydroxypiperidine-1-carboxylate was consumed completely and the desired mass was detected. The mixture was concentrated under reduced pressure to give the title compound (60.0 mg, crude, TFA) as a yellow oil. LCMS (ES⁺): m/z 433.3 [M+H]⁺.

Synthesis of trans-4-((5-fluoro-4-(3-(2-oxopyridin-1 (2H)-yl)phenyl)pyrimidin-2-yl)amino)cyclohexyl 4-(2-(4-(4-((2,6-dioxopiperidin-3-yl)amino)-2-fluorophenyl)piperidin-1-yl)ethyl)-4-hydroxypiperidine-1-carboxylate (Compound 641). To a solution of 3-((3-fluoro-4-(1-(2-(4-hydroxypiperidin-4-yl)ethyl)piperidin-4-yl)phenyl)amino)piperidine-2,6-dione (50.0 mg, 91.5 μmol, 1.00 eq, TFA) in THF (3 mL) were added DIEA (59.3 mg, 275 μmol, 79.8 μL, 5.00 eq) and trans-4-((5-fluoro-4-(3-(2-oxopyridin-1 (2H)-yl)phenyl)pyrimidin-2-yl)amino)cyclohexyl (4-nitrophenyl) carbonate (34.9 mg, 64.0 μmol, 0.700 eq) at 25 °C, the resulting mixture was stirred at 25 °C for 4 h. LCMS showed 3-((3-fluoro-4-(1-(2-(4-hydroxypiperidin-4-yl)ethyl)piperidin-4-yl)phenyl)amino)piperidine-2,6-dione was consumed completely and the desired mass was detected. The mixture was diluted with H₂O (6 mL) and then extracted with EtOAc (6 mL*3). The combined organic layers were dried over Na₂SO₄, filtered and the filtrate was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 40%-70% B over 8.0 min) to give the compound (Compound 641) (3.10 mg, 3.70 μmol, 4.04% yield, 100% purity) as a yellow solid. LCMS (ES⁺): m/z 839.3 [M+H]⁺.

### Example 634. Synthesis of trans-4-((5-fluoro-4-(3-(2-oxopyridin-1 (2H)-yl)phenyl)pyrimidin-2-yl)amino)cyclohexyl 4-(2-(4-(4-((2,6-dioxopiperidin-3-yl)amino)-2-fluorophenyl)piperidin-1-yl)-2-oxoethyl)-4-hydroxypiperidine-1-carboxylate (Compound 663)

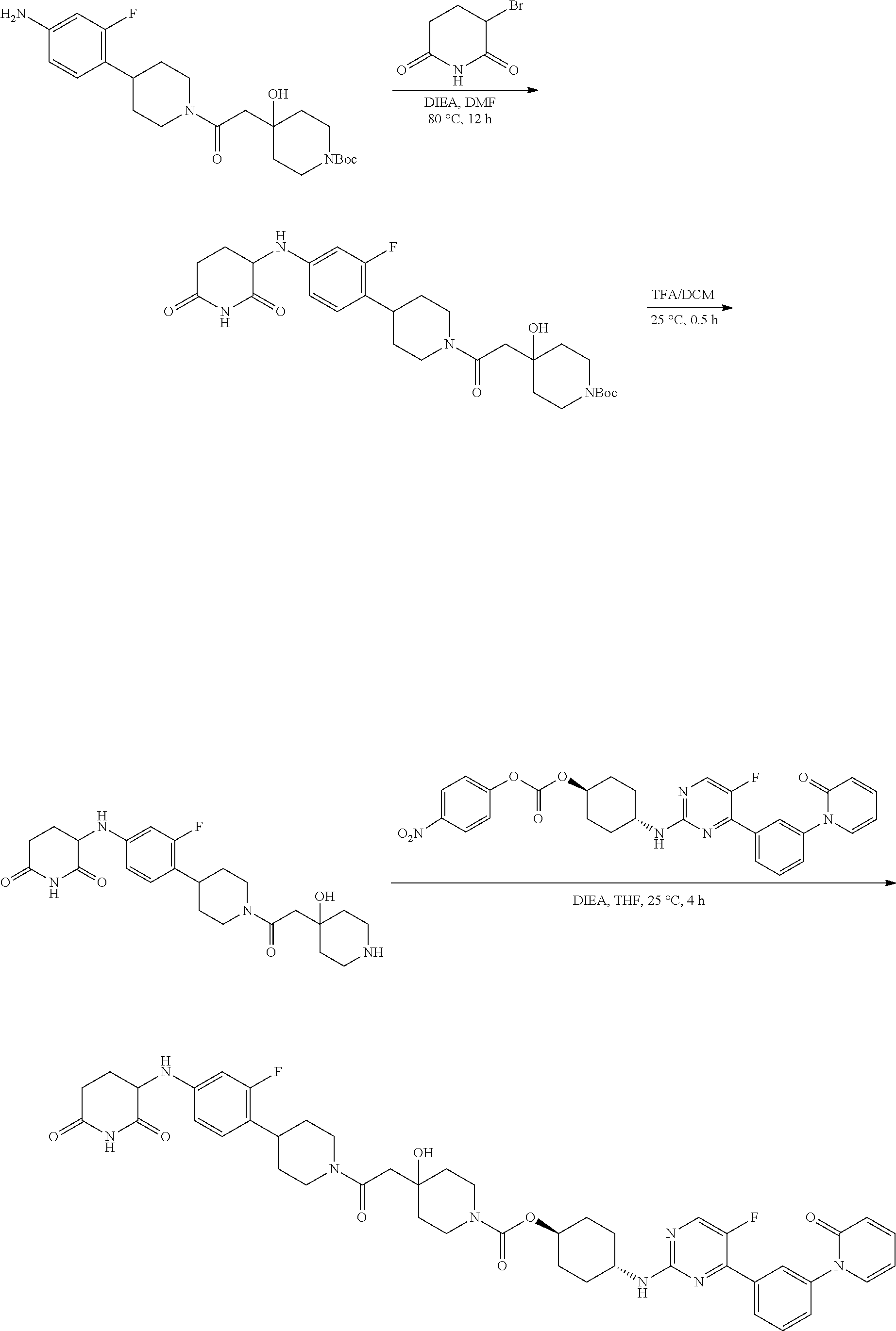

Synthesis of tert-butyl 4-(2-(4-(4-((2,6-dioxopiperidin-3-yl)amino)-2-fluorophenyl)piperidin-1-yl)-2-oxoethyl)-4-hydroxypiperidine-1-carboxylate. To a solution of tert-butyl 4-(2-(4-(4-amino-2-fluorophenyl)piperidin-1-yl)-2-oxoethyl)-4-hydroxypiperidine-1-carboxylate (1.50 g, 3.44 mmol, 1.00 eq) in DMF (35 mL) were added DIEA (890 mg, 6.89 mmol, 1.20 mL, 2.00 eq) and 3-bromopiperidine-2,6-dione (1.98 g, 10.3 mmol, 3.00 eq) at 25 °C, the mixture was stirred at 80 °C for 12 h. LCMS showed tert-butyl 4-(2-(4-(4-amino-2-fluorophenyl)piperidin-1-yl)-2-oxoethyl)-4-hydroxypiperidine-1-carboxylate was consumed and the desired mass was detected. H₂O (50 mL) was added to the mixture and the mixture was extracted with EtOAc (50 mL*3). The combined organic layers were washed with brine (80 mL*3), dried over Na₂SO₄, filtered and the filtrate was concentrated under reduced pressure to give the title compound (800 mg, 1.46 mmol, 42.5% yield) as a black solid. LCMS (ES⁺): m/z 491.3 [M+H−56]+.

Synthesis of 3-((3-fluoro-4-(1-(2-(4-hydroxypiperidin-4-yl)acetyl)piperidin-4-yl)phenyl)amino)piperidine-2,6-dione. A solution of tert-butyl 4-(2-(4-(4-((2,6-dioxopiperidin-3-yl)amino)-2-fluorophenyl)piperidin-1-yl)-2-oxoethyl)-4-hydroxypiperidine-1-carboxylate (300 mg, 549 μmol, 1.00 eq) in DCM (3 mL) and TFA (1 mL) was stirred at 25 °C for 0.5 h. LCMS showed tert-butyl 4-(2-(4-(4-((2,6-dioxopiperidin-3-yl)amino)-2-fluorophenyl)piperidin-1-yl)-2-oxoethyl)-4-hydroxypiperidine-1-carboxylate was consumed completely and the desired mass was detected. The mixture was concentrated under reduced pressure to give the title compound (300 mg, crude, TFA) as a black oil. LCMS (ES⁺): m/z 447.3 [M+H]⁺.

Synthesis of trans-4-((5-fluoro-4-(3-(2-oxopyridin-1 (2H)-yl)phenyl)pyrimidin-2-yl)amino)cyclohexyl 4-(2-(4-(4-((2,6-dioxopiperidin-3-yl)amino)-2-fluorophenyl)piperidin-1-yl)-2-oxoethyl)-4-hydroxypiperidine-1-carboxylate (Compound 663). To a solution of 3-((3-fluoro-4-(1-(2-(4-hydroxypiperidin-4-yl)acetyl)piperidin-4-yl)phenyl)amino)piperidine-2,6-dione (300 mg, 535 μmol, 1.00 eq, TFA) in THF (4 mL) were added DIEA (345 mg, 2.68 mmol, 465 μL, 5.00 eq) and trans-4-((5-fluoro-4-(3-(2-oxopyridin-1 (2H)-yl)phenyl)pyrimidin-2-yl)amino)cyclohexyl (4-nitrophenyl) carbonate (146 mg, 268 μmol, 0.500 eq) at 25 °C, the resulting mixture was stirred at 25 °C for 4 h. LCMS showed 3-((3-fluoro-4-(1-(2-(4-hydroxypiperidin-4-yl)acetyl)piperidin-4-yl)phenyl)amino)piperidine-2,6-dione was consumed completely and the desired mass was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 25%-55% B over 8.0 min) to give the title compound (Compound 663) (29.5 mg, 33.9 μmol, 6.33% yield, 97.9% purity) as a white solid. LCMS (ES⁺): m/z 853.4 [M+H]⁺.

### Example 635. Synthesis of trans-2-((1r,4R)-4-((4-(4-((2,6-dioxopiperidin-3-yl)amino)-2-fluorophenyl)piperazin-1-yl)methyl)cyclohexyl)-N-((1r,4R)-4-((5-fluoro-4-(3-(2-oxo-1,2-dihydropyridin-3-yl)phenyl)pyrimidin-2-yl)amino)cyclohexyl)acetamide (Compound 703)

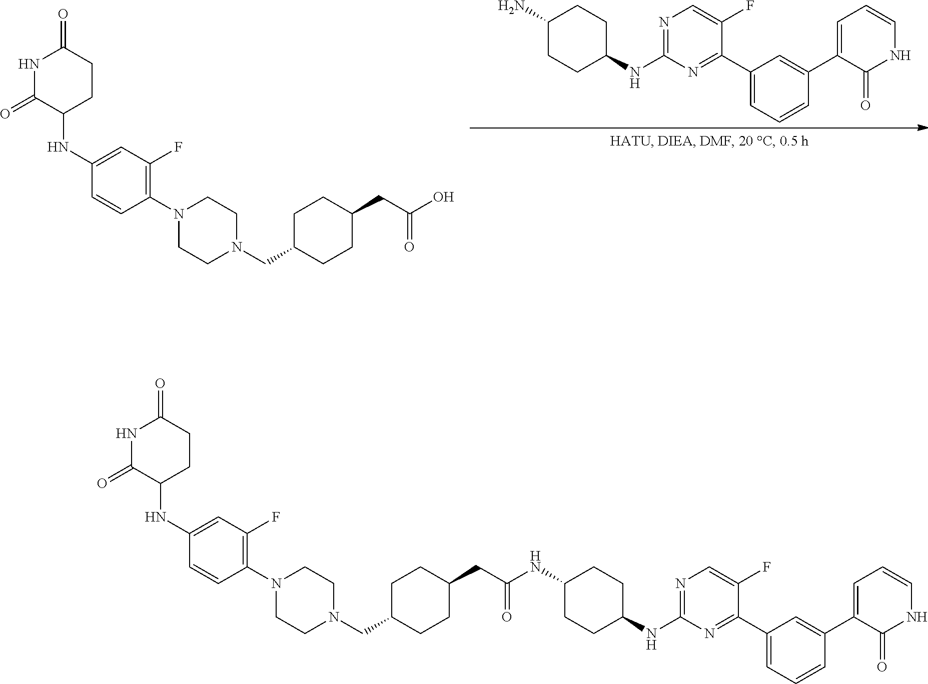 [SMILES: O=C(O)C[C@H]1CC[C@H](CN2CCN(c3ccc(NC4CCC(=O)NC4=O)cc3F)CC2)CC1 ; N[C@H]1CC[C@H](Nc2ncc(F)c(-c3cccc(-c4ccc[nH]c4=O)c3)n2)CC1 ; O=C1CCC(Nc2ccc(N3CCN(C[C@H]4CC[C@H](CC(=O)N[C@H]5CC[C@H](Nc6ncc(F)c(-c7cccc(-c8ccc[nH]c8=O)c7)n6)CC5)CC4)CC3)c(F)c2)C(=O)N1]

Synthesis of trans-2-((1r,4R)-4-((4-(4-((2,6-dioxopiperidin-3-yl)amino)-2-fluorophenyl)piperazin-1-yl)methyl)cyclohexyl)-N-((1r,4R)-4-((5-fluoro-4-(3-(2-oxo-1,2-dihydropyridin-3-yl)phenyl)pyrimidin-2-yl)amino)cyclohexyl)acetamide (Compound 703). To solution of trans-2-(4-((4-(4-((2,6-dioxopiperidin-3-yl)amino)-2-fluorophenyl)piperazin-1-yl)methyl)cyclohexyl)acetic acid (160 mg, 347 μmol, 1.00 eq) and trans-3-(3-(2-((4-aminocyclohexyl)amino)-5-fluoropyrimidin-4-yl)phenyl)pyridin-2 (1H)-one (116 mg, 278 μmol, 0.800 eq, HCl) in DMF (4 mL) were added HATU (66.1 mg, 174 μmol, 0.500 eq) and DIEA (225 mg, 1.73 mmol, 303 μL, 5.00 eq) at 20 °C, the mixture was stirred at 20 °C for 0.5 h. LCMS showed trans-2-(4-((4-(4-((2,6-dioxopiperidin-3-yl)amino)-2-fluorophenyl)piperazin-1-yl)methyl)cyclohexyl)acetic acid was consumed and the desired target MS was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 21%-51% B over 8.0 min) to give the title compound (Compound 703) (51.5 mg, 58.0 μmol, 16.7% yield, 92.6% purity) as a pale yellow solid. LCMS (ES⁺): m/z 822.4 [M+H]⁺

### Example 636. Synthesis of 3-((3-fluoro-4-(1-(((trans)-4-(4-((5-fluoro-4-(3-(2-oxo-1,2-dihydropyridin-3-yl)phenyl)pyrimidin-2-yl)amino)piperidine-1-carbonyl)cyclohexyl)methyl)piperidin-4-yl)phenyl)amino)piperidine-2,6-dione (Compound 654)

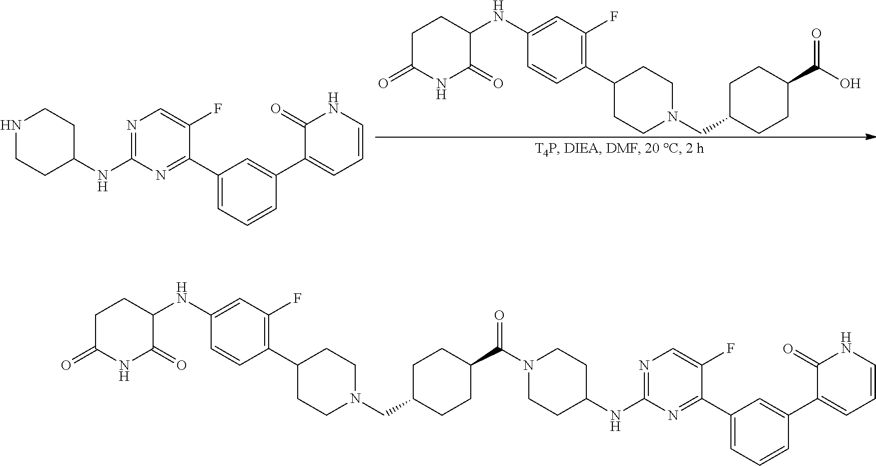 [SMILES: O=c1[nH]cccc1-c1cccc(-c2nc(NC3CCNCC3)ncc2F)c1 ; O=C1CCC(Nc2ccc(C3CCN(C[C@H]4CC[C@H](C(=O)O)CC4)CC3)c(F)c2)C(=O)N1 ; O=C1CCC(Nc2ccc(C3CCN(C[C@H]4CC[C@H](C(=O)N5CCC(Nc6ncc(F)c(-c7cccc(-c8ccc[nH]c8=O)c7)n6)CC5)CC4)CC3)c(F)c2)C(=O)N1]

Synthesis of 3-((3-fluoro-4-(1-(((1r,4r)-4-(4-((5-fluoro-4-(3-(2-oxo-1,2-dihydropyridin-3-yl)phenyl)pyrimidin-2-yl)amino)piperidine-1-carbonyl)cyclohexyl)methyl)piperidin-4-yl)phenyl)amino)piperidine-2,6-dione (Compound 654). To a solution of 3-(3-(5-fluoro-2-(piperidin-4-ylamino)pyrimidin-4-yl)phenyl)pyridin-2 (1H)-one (200 mg, 497 μmol, 1.00 eq, HCl) in DMF (3 mL) was added trans-4-((4-(4-((2,6-dioxopiperidin-3-yl)amino)-2-fluorophenyl)piperidin-1-yl)methyl)cyclohexane-1-carboxylic acid (221.73 mg, 396.26 μmol, 0.796 eq, TFA), DIEA (128 mg, 995 μmol, 173 μL, 2.00 eq) and T₄P (717 mg, 995 μmol, 109 μL, 50.0% purity, 2.00 eq) at 20 °C. The mixture was stirred at 20 °C for 2 h. LC-MS showed the desired mass was detected. The reaction mixture was concentrated under high vacuum. The residue was purified by prep-HPLC (Phenomenex luna C18 100*40 mm*3 um; mobile phase: [H₂O (0.2% FA)-ACN]; gradient: 5%-40% B over 8.0 min). to give the title compound (Compound 654) (21.8 mg, 26.1 μmol, 5.24% yield, 96.0% purity, 0.2 FA) as a pale yellow solid. LCMS (ES⁺): m/z 793.4 [M+H]⁺.

### Example 637. Synthesis of trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(2-oxopiperidin-1-yl)phenyl)pyrimidin-2-yl)amino)cyclohexane-1-carboxamide (Compound 701)

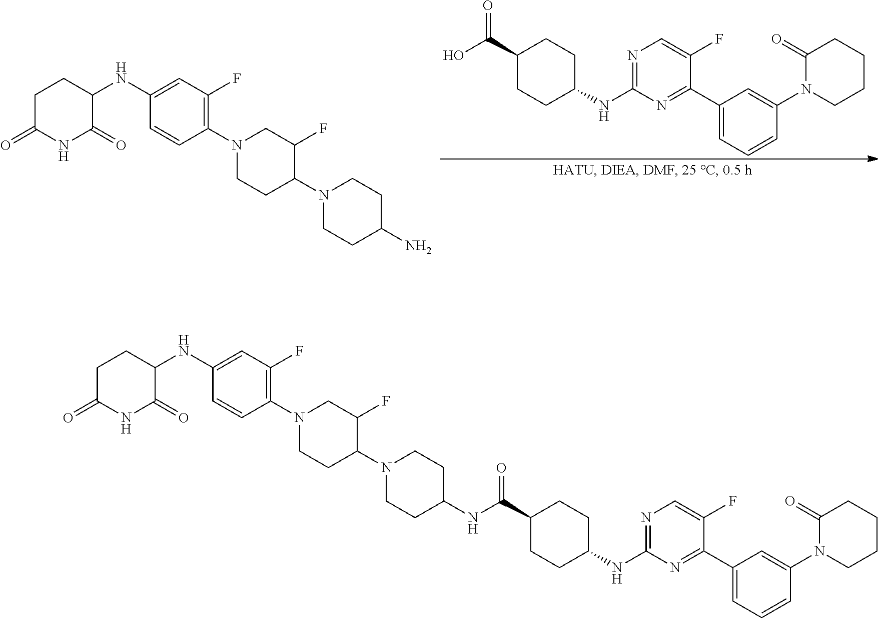 [SMILES: NC1CCN(C2CCN(c3ccc(NC4CCC(=O)NC4=O)cc3F)CC2F)CC1 ; O=C1CCCCN1c1cccc(-c2nc(N[C@H]3CC[C@H](C(=O)O)CC3)ncc2F)c1 ; O=C1CCC(Nc2ccc(N3CCC(N4CCC(NC(=O)[C@H]5CC[C@H](Nc6ncc(F)c(-c7cccc(N8CCCCC8=O)c7)n6)CC5)CC4)C(F)C3)c(F)c2)C(=O)N1]

Synthesis of trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(2-oxopiperidin-1-yl)phenyl)pyrimidin-2-yl)amino)cyclohexane-1-carboxamide (Compound 701). To a solution of 3-((4-(4-amino-3′-fluoro-[1,4′-bipiperidin]-1′-yl)-3-fluorophenyl)amino)piperidine-2,6-dione (200 mg, 373 μmol, 1.00 eq, TFA) and trans-4-((5-fluoro-4-(3-(2-oxopiperidin-1-yl)phenyl)pyrimidin-2-yl)amino)cyclohexane-1-carboxylic acid (123 mg, 299 μmol, 0.800 eq) in DMF (5 mL) were added HATU (49.7 mg, 131 μmol, 0.350 eq) and DIEA (241 mg, 1.87 mmol, 325 μL, 5.00 eq) at 25 °C, then the mixture was stirred at 25 °C for 0.5 h. LCMS showed 3-((4-(4-amino-3′-fluoro-[1,4′-bipiperidin]-1′-yl)-3-fluorophenyl)amino)piperidine-2,6-dione was consumed and the desired target MS was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 40%-70% B over 8.0 min) to give the title compound (Compound 701) (22.9 mg, 25.9 μmol, 6.94% yield, 92.3% purity) as a pale yellow solid. LCMS (ES+): m/z 816.5 [M+H]⁺.

### Example 638. Synthesis of 3-((3-fluoro-4-(1-((trans-4-(4-((5-fluoro-4-(3-(2-oxopiperidin-1-yl)phenyl)pyrimidin-2-yl)amino)piperidine-1-carbonyl)cyclohexyl)methyl)piperidin-4-yl)phenyl)amino)piperidine-2,6-dione (Compound 685)

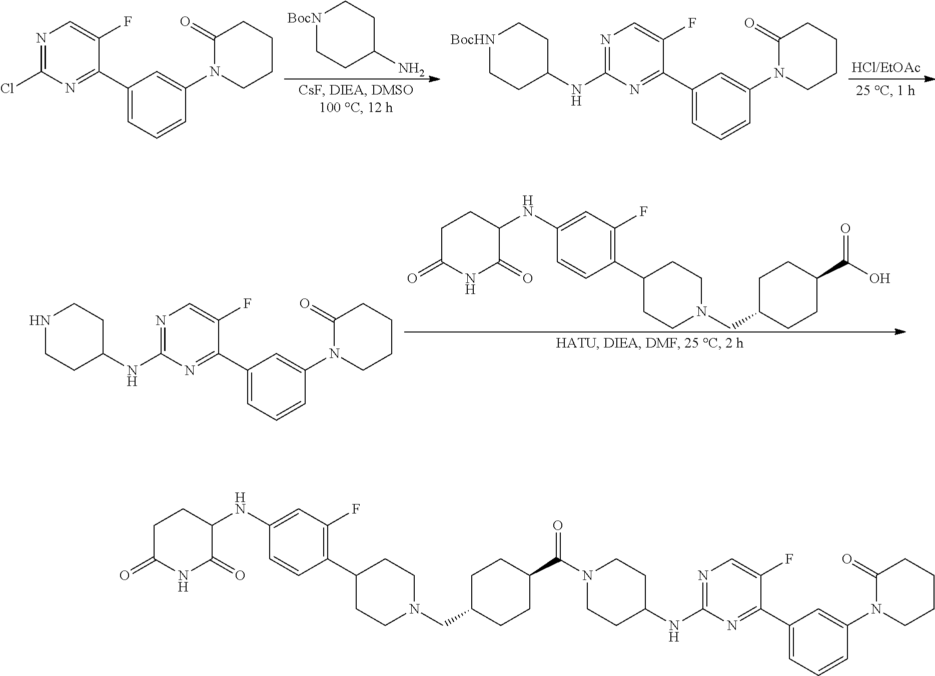

Synthesis of tert-butyl 4-((5-fluoro-4-(3-(2-oxopiperidin-1-yl)phenyl)pyrimidin-2-yl)amino)piperidine-1-carboxylate. To a solution of 1-[3-(2-chloro-5-fluoro-pyrimidin-4-yl)phenyl]piperidin-2-one (5.00 g, 16.3 mmol, 1.00 eq) and tert-butyl 4-aminopiperidine-1-carboxylate (6.55 g, 32.7 mmol, 2.00 eq) in DMSO (50 mL) was added DIEA (4.23 g, 32.7 mmol, 5.70 mL, 2.00 eq) and CsF (4.97 g, 32.7 mmol, 2.00 eq) at 25 °C. The mixture was stirred at 100 °C for 12 h. LC-MS showed the desired mass was detected. The reaction mixture was quenched by addition H₂O 100 mL, and then extracted with EtOAc 300 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (4.00 g, 8.35 mmol, 51.0% yield, 98.0% purity) as a white solid. LCMS (ES+): m/z 370.3 [M+H−100]+.

Synthesis of 1-(3-(5-fluoro-2-(piperidin-4-ylamino)pyrimidin-4-yl)phenyl)piperidin-2-one. A mixture of tert-butyl 4-[[5-fluoro-4-[3-(2-oxo-1-piperidyl)phenyl]pyrimidin-2-yl]amino]piperidine-1-carboxylate (1.00 g, 2.13 mmol, 1.00 eq) in HCl/EtOAc (4.00 M, 20.0 mL, 37.5 eq) was stirred at 25 °C for 1 h. LC-MS showed the desired mass was detected. The reaction mixture was concentrated under reduced pressure to give the title compound (1.00 g, crude, HCl) as a yellow solid. LCMS (ES+): m/z 370.1 [M+H]+.

Synthesis of 3-((3-fluoro-4-(1-(((trans)-4-(4-((5-fluoro-4-(3-(2-oxopiperidin-1-yl)phenyl)pyrimidin-2-yl)amino)piperidine-1-carbonyl)cyclohexyl)methyl)piperidin-4-yl)phenyl)amino)piperidine-2,6-dione (Compound 685). To a solution of 1-(3-(5-fluoro-2-(piperidin-4-ylamino)pyrimidin-4-yl)phenyl)piperidin-2-one (100 mg, 178 μmol, 1.00 eq, TFA) and trans-4-((4-(4-((2,6-dioxopiperidin-3-yl)amino)-2-fluorophenyl)piperidin-1-yl)methyl)cyclohexane-1-carboxylic acid (66.0 mg, 162 μmol, 0.910 eq, HCl) in DMF (2 mL) was added DIEA (46.1 mg, 357 μmol, 62.2 μL, 2.00 eq) and HATU (135 mg, 357 μmol, 2.00 eq) at 25 °C. The mixture was stirred at 25 °C for 2 h. LC-MS showed the desired mass was detected. The reaction mixture was concentrated under reduced pressure. The residue was purified by prep-HPLC (column: Phenomenex Luna C18 100*30 mm*3 um; mobile phase: [H₂O (0.2% FA)-ACN]; gradient: 15%-45% B over 8.0 min) to give the title compound (Compound 685) (15.7 mg, 19.3 μmol, 10.8% yield, 100% purity, 0.350 FA) as a pale yellow solid. LCMS (ES+): m/z 797.4 [M+H]+.

### Example 639. Synthesis of trans-4-((5-fluoro-4-(3-(2-oxopiperidin-1-yl)phenyl)pyrimidin-2-yl)amino)cyclohexyl 4-(4-((2,6-dioxopiperidin-3-yl)amino)-2-fluorophenyl)-[1,4′-bipiperidine]-1′-carboxylate (Compound 678)

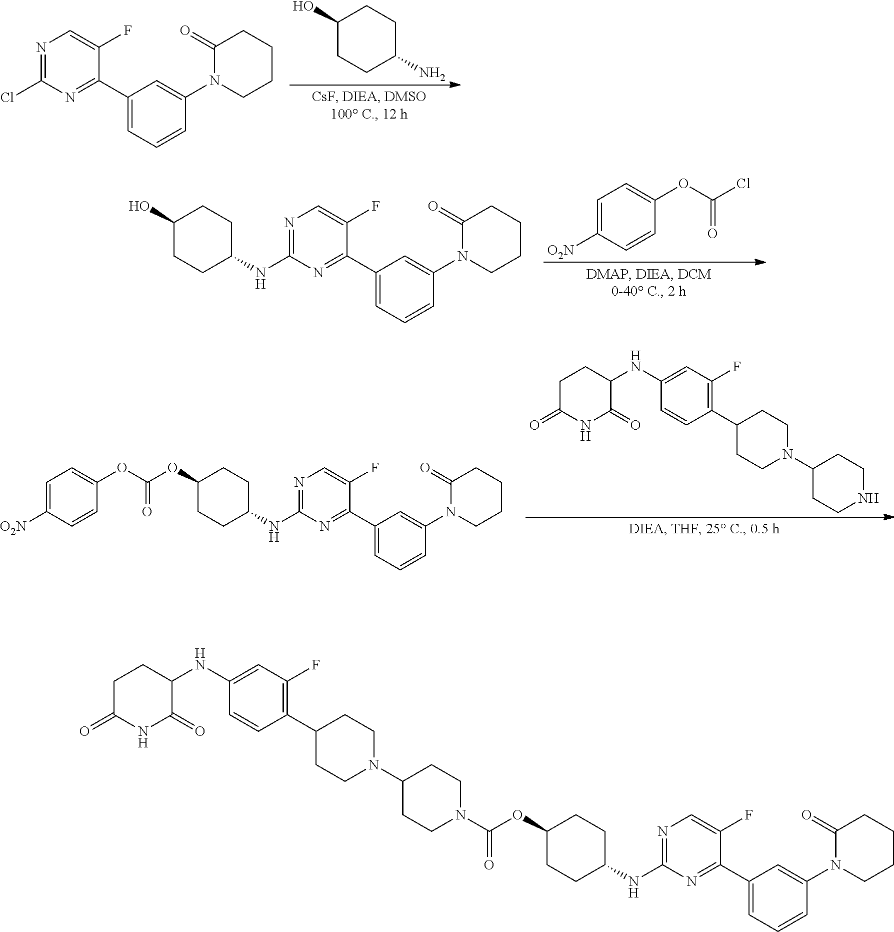

Synthesis of trans-1-(3-(5-fluoro-2-((-4-hydroxycyclohexyl)amino)pyrimidin-4-yl)phenyl)piperidin-2-one. To a solution of 1-(3-(2-chloro-5-fluoropyrimidin-4-yl)phenyl)piperidin-2-one (3.00 g, 9.81 mmol, 1.00 eq) in DMSO (100 mL) were added trans-4-aminocyclohexan-1-ol (1.70 g, 14.7 mmol, 1.50 eq), CsF (2.24 g, 14.7 mmol, 1.50 eq) and DIEA (3.80 g, 29.4 mmol, 5.13 mL, 3.00 eq) at 25 °C. The mixture was stirred at 100 °C for 12 h. LC-MS showed 1-(3-(2-chloro-5-fluoropyrimidin-4-yl)phenyl)piperidin-2-one was consumed completely and the desired mass was detected. To the reaction mixture was added H₂O (20 mL) at 25 °C, then it was filtered. The filter cake was dried reduced pressure to give the crude product. The crude product was purified by column chromatography (SiO₂, Petroleum ether:Ethyl acetate=10:1 to 5:1) to give the title compound (2.48 g, 6.45 mmol, 65.7% yield) as a brown solid. LCMS (ES⁺): m/z 385.2 [M+H]⁺.

Synthesis of trans-4-((5-fluoro-4-(3-(2-oxopiperidin-1-yl)phenyl)pyrimidin-2-yl)amino)cyclohexyl (4-nitrophenyl) carbonate. To a solution of trans-1-(3-(5-fluoro-2-((-4-hydroxycyclohexyl)amino)pyrimidin-4-yl)phenyl)piperidin-2-one (600 mg, 1.56 mmol, 1.00 eq) in DCM (30 mL) was added (4-nitrophenyl) carbonochloridate (1.57 g, 7.80 mmol, 5.00 eq) at 25 °C. Then to the mixture were added DMAP (286 mg, 2.34 mmol, 1.50 eq) and DIEA (605 mg, 4.68 mmol, 816 μL, 3.00 eq) at 0 °C. The mixture was stirred at 40 °C for 2 h. LCMS showed trans-1-(3-(5-fluoro-2-((-4-hydroxycyclohexyl)amino)pyrimidin-4-yl)phenyl)piperidin-2-one was consumed completely, and the desired mass was detected. The reaction mixture was concentrated under reduced pressure to give a residue. The residue was purified by column chromatography (SiO₂, Ethyl acetate:Petroleum ether=1:1 to 2:1) to give the title compound (484 mg, 881 μmol, 56.4% yield) as a brown solid. LCMS (ES⁺): m/z 550.2 [M+H]⁺.

Synthesis of trans-4-((5-fluoro-4-(3-(2-oxopiperidin-1-yl)phenyl)pyrimidin-2-yl)amino)cyclohexyl 4-(4-((2,6-dioxopiperidin-3-yl)amino)-2-fluorophenyl)-[1,4′-bipiperidine]-1′-carboxylate (Compound 678). To a solution of trans-4-((5-fluoro-4-(3-(2-oxopiperidin-1-yl)phenyl)pyrimidin-2-yl)amino)cyclohexyl (4-nitrophenyl) carbonate (161 mg, 294 μmol, 0.700 eq) in THF (4 mL) were added DIEA (272 mg, 2.10 mmol, 365 μL, 5.00 eq) and 3-((4-([1,4′-bipiperidin]-4-yl)-3-fluorophenyl)amino)piperidine-2,6-dione (211 mg, 420 μmol, 1.00 eq, TFA) at 25 °C. The mixture was stirred at 25 °C for 0.5 h. LCMS showed trans-4-((5-fluoro-4-(3-(2-oxopiperidin-1-yl)phenyl)pyrimidin-2-yl)amino)cyclohexyl (4-nitrophenyl) carbonate was consumed completely, and the desired mass was detected. The reaction mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 15%-55% B over 8.0 min) to give the title compound (Compound 678) (78.1 mg, 96.7 μmol, 23.0% yield, 98.9% purity) as a white solid. LCMS (ES⁺): m/z 799.4 [M+H]⁺.

### Example 640. Synthesis of trans-4-((5-fluoro-4-(3-(2-oxopiperidin-1-yl)phenyl)pyrimidin-2-yl)amino)cyclohexyl 4-(4-(4-((2,6-dioxopiperidin-3-yl)amino)-2-fluorophenyl)piperazin-1-yl)piperidine-1-carboxylate (Compound 675)

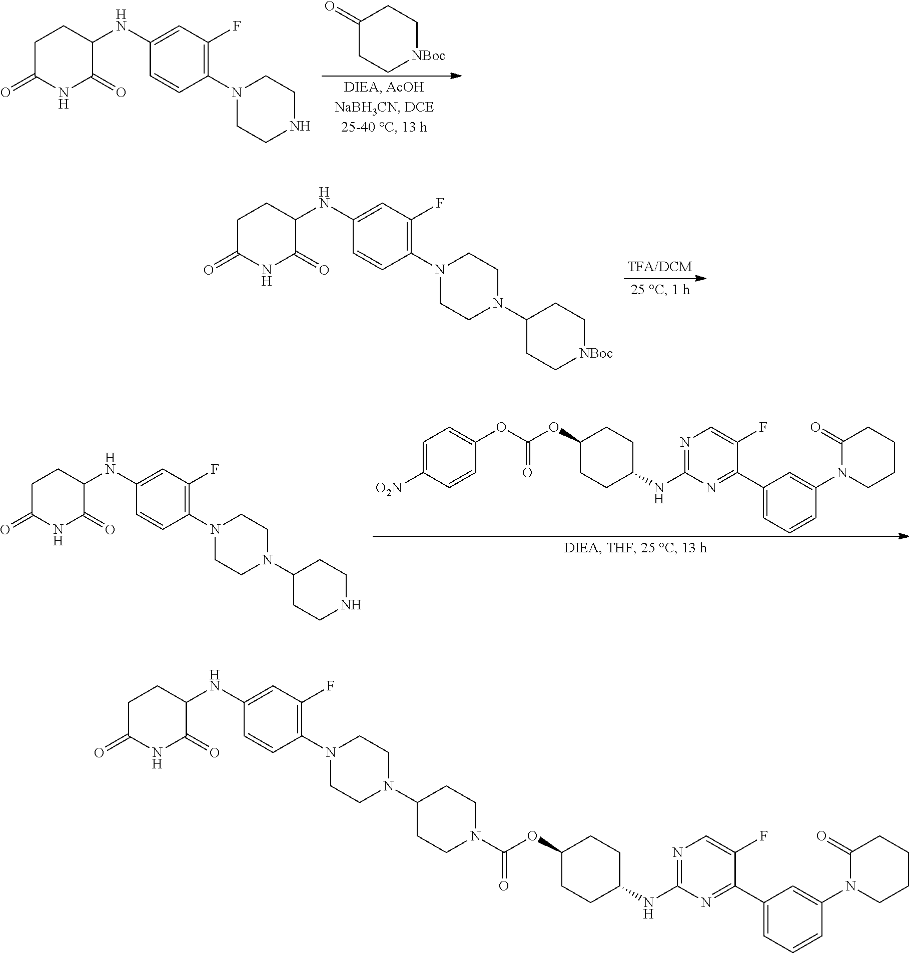 [SMILES: O=C1CCC(Nc2ccc(N3CCNCC3)c(F)c2)C(=O)N1 ; CC(C)(C)OC(=O)N1CCC(=O)CC1 ; CC(C)(C)OC(=O)N1CCC(N2CCN(c3ccc(NC4CCC(=O)NC4=O)cc3F)CC2)CC1 ; O=C1CCC(Nc2ccc(N3CCN(C4CCNCC4)CC3)c(F)c2)C(=O)N1 ; O=C(Oc1ccc([N+](=O)[O-])cc1)O[C@H]1CC[C@H](Nc2ncc(F)c(-c3cccc(N4CCCCC4=O)c3)n2)CC1 ; O=C1CCC(Nc2ccc(N3CCN(C4CCN(C(=O)O[C@H]5CC[C@H](Nc6ncc(F)c(-c7cccc(N8CCCCC8=O)c7)n6)CC5)CC4)CC3)c(F)c2)C(=O)N1]

Synthesis of tert-butyl 4-(4-(4-((2,6-dioxopiperidin-3-yl)amino)-2-fluorophenyl)piperazin-1-yl)piperidine-1-carboxylate. To a solution of 3-((3-fluoro-4-(piperazin-1-yl)phenyl)amino)piperidine-2,6-dione (6.00 g, 14.3 mmol, 1.00 eq, TFA) in DCE (200 mL) was added DIEA at 25 °C until the pH had reached 8. The mixture was stirred at 25 °C for 0.5 h. To the mixture were added CH3COOH (4.29 g, 71.5 mmol, 4.09 mL, 5.00 eq) and tert-butyl 4-oxopiperidine-1-carboxylate (14.2 g, 71.4 mmol, 5.00 eq) at 25 °C, pH was 5 now. The mixture was stirred at 40 °C for 0.5 h. Then NaBH₃CN (2.69 g, 42.8 mmol, 3.00 eq) was added to the mixture at 25 °C. The mixture was stirred at 40 °C for 12 h. LCMS showed 3-((3-fluoro-4-(piperazin-1-yl)phenyl)amino)piperidine-2,6-dione was consumed and the desired mass was detected. The mixture was diluted with H₂O (200 mL) and then extracted with DCM (100 mL*3). The combined organic layers were dried over Na₂SO₄, filtered and the filtrate was concentrated under reduced pressure to give a residue. The residue was purified by MPLC (SiO₂, PE/EtOAc=1/0 to 1/1) to give the title compound (1.50 g, 3.06 mmol, 21.5% yield) as a green solid. LCMS (ES⁺): m/z 490.3 [M+H]⁺.

Synthesis of 3-((3-fluoro-4-(4-(piperidin-4-yl)piperazin-1-yl)phenyl)amino)piperidine-2,6-dione. A solution of tert-butyl 4-(4-(4-((2,6-dioxopiperidin-3-yl)amino)-2-fluorophenyl)piperazin-1-yl)piperidine-1-carboxylate (200 mg, 409 μmol, 1.00 eq) in DCM (3 mL) and TFA (1 mL) was stirred at 25 °C for 1 h. LCMS showed tert-butyl 4-(4-(4-((2,6-dioxopiperidin-3-yl)amino)-2-fluorophenyl)piperazin-1-yl)piperidine-1-carboxylate was consumed completely and the desired mass was detected. The mixture was concentrated under reduced pressure to give the title compound (200 mg, crude, TFA) as a yellow oil. LCMS (ES⁺): m/z 390.3 [M+H]⁺.

Synthesis of trans-4-((5-fluoro-4-(3-(2-oxopiperidin-1-yl)phenyl)pyrimidin-2-yl)amino)cyclohexyl 4-(4-(4-((2,6-dioxopiperidin-3-yl)amino)-2-fluorophenyl)piperazin-1-yl)piperidine-1-carboxylate (Compound 675). To a stirred solution of 3-[3-fluoro-4-[4-(4-piperidyl)piperazin-1-yl]anilino]piperidine-2,6-dione (200 mg, 397 μmol, 1.00 eq, TFA) and DIEA (257 mg, 1.98 mmol, 347 μL, 5.00 eq) in THF (4 mL) was added trans-4-((5-fluoro-4-(3-(2-oxopiperidin-1-yl)phenyl)pyrimidin-2-yl)amino)cyclohexyl (4-nitrophenyl) carbonate (153 mg, 278 μmol, 0.700 eq) at 25 °C. The resulting mixture was stirred at 25 °C for 13 h. LCMS showed 3-[3-fluoro-4-[4-(4-piperidyl)piperazin-1-yl]anilino]piperidine-2,6-dione was consumed and the desired mass was detected. The mixture was concentrated under reduced pressure to give a residue. The residue was purified by prep-HPLC (column: Phenomenex Gemini-NX 80*30 mm*3 um; mobile phase: [H₂O (10 mM NH₄HCO₃)-ACN]; gradient: 30%-60% B over 8.0 min) to give the title compound (Compound 675) (67.0 mg, 76.4 μmol, 19.2% yield, 91.2% purity) as a pale yellow solid. LCMS (ES⁺): m/z 800.4 [M+H]⁺.

### Example 641. Synthesis of trans-N-(1′-(4-((2,6-dioxopiperidin-3-yl)amino)-2-fluorophenyl)-3′-fluoro-[1,4′-bipiperidin]-4-yl)-4-((5-fluoro-4-(3-(piperidin-1-yl)phenyl)pyrimidin-2-yl)amino)cyclohexane-1-carboxamide (Compound 700)

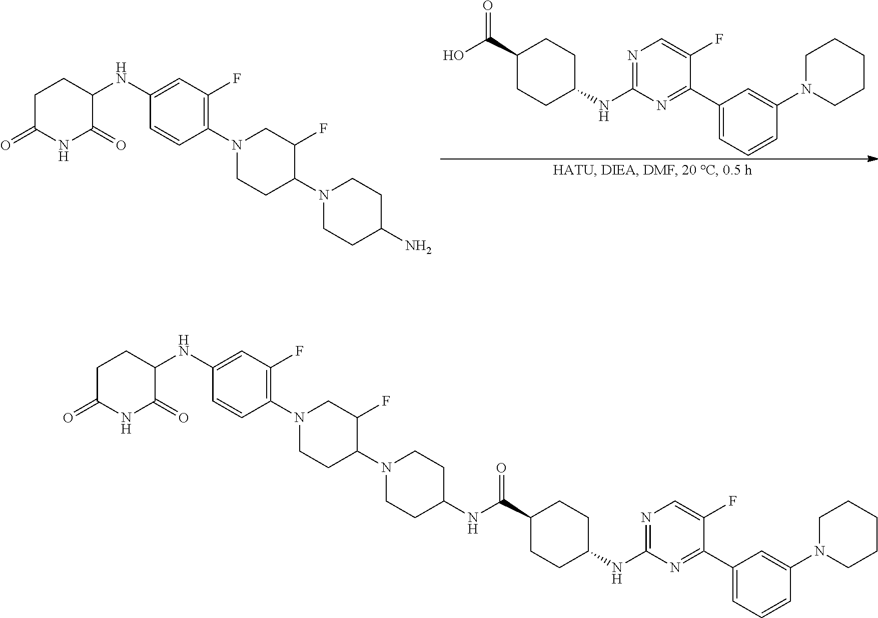 [SMILES: NC1CCN(C2CCN(c3ccc(NC4CCC(=O)NC4=O)cc3F)CC2F)CC1 ; O=C(O)[C@H]1CC[C@H](Nc2ncc(F)c(-c3cccc(N4CCCCC4)c3)n2)CC1 ; O=C1CCC(Nc2ccc(N3CCC(N4CCC(NC(=O)[C@H]5CC[C@H](Nc6ncc(F)c(-c7cccc(N8CCCCC8)c7)n6)CC5)CC4)C(F)C3)c(F)c2)C(=O)N1]

Synthesis of trans-N-(1′-(4-((2,6-dioxopiperidin-3-yl)amino)-2-fluorophenyl)-3′ fluoro-[1,4′-bipiperidin]-4-yl)-4-((5-fluoro-4-(3-(piperidin-1-yl)phenyl)pyrimidin-2-yl)amino)cyclohexane-1-carboxamide (Compound 700). To a solution of trans-4-((5-fluoro-4-(3-(piperidin-1-yl)phenyl)pyrimidin-2-yl)amino)cyclohexane-1-carboxylic acid (59.5 mg, 149 μmol, 0.400 eq) in DMF (3 mL) were added HATU (49.7 mg, 131 μmol, 0.350 eq) and DIEA (242 mg, 1.87 mmol, 325 μL, 5.00 eq) at 20 °C, then 3-((4-(4-amino-3′-fluoro-[1,4′-bipiperidin]-1′-yl)-3-fluorophenyl)amino)piperidine-2,6-dione (200 mg, 373 μmol, 1.00 eq, TFA) was added to the mixture, the mixture was stirred at 20 °C for 0.5 h. LCMS showed 3-((4-(4-amino-3′-fluoro-[1,4′-bipiperidin]-1′-yl)-3-fluorophenyl)amino)piperidine-2,6-dione was consumed and the desired mass was detected. The mixture was concentrated under reduced pressure to give a residue. The residue was purified by prep-HPLC (column: Phenomenex Gemini-NX 80*40 mm*3 um; mobile phase: [1H₂O (10 mM NH₄HCO₃)-ACN]; gradient: 40%-70% B over 8.0 min) to give the title compound (Compound 700) (23.7 mg, 29.3 μmol, 7.83% yield, 99.0% purity) as a white solid. LCMS (ES⁺): m/z 802.5 [M+H]⁺

### Example 642. Synthesis of trans-3-((3-fluoro-4-(1-((4-(4-((5-fluoro-4-(3-(piperidin-1-yl)phenyl)pyrimidin-2-yl)amino)piperidine-1-carbonyl)cyclohexyl)methyl)piperidin-4-yl)phenyl)amino)piperidine-2,6-dione (Compound 682)

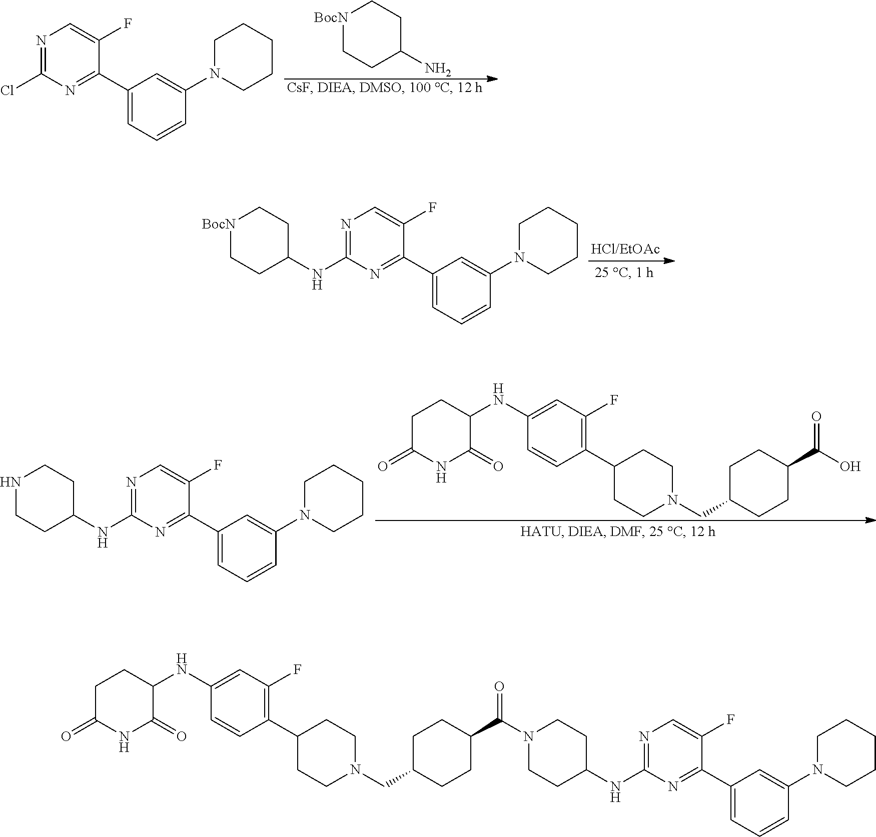

Synthesis of tert-butyl 4-((5-fluoro-4-(3-(piperidin-1-yl)phenyl)pyrimidin-2-yl)amino)piperidine-1-carboxylate. To a solution of 2-chloro-5-fluoro-4-[3-(1-piperidyl)phenyl]pyrimidine (3.00 g, 10.2 mmol, 1.00 eq) in DMSO (20 mL) was added CsF (1.56 g, 10.2 mmol, 1.00 eq), tert-butyl 4-aminopiperidine-1-carboxylate (2.06 g, 10.2 mmol, 1.00 eq) and DIEA (7.82 g, 20.5 mmol, 2.00 eq) at 25 °C. The mixture was stirred at 100 °C for 12 h. LC-MS showed the desired mass was detected. The reaction was quenched by addition H₂O 80 mL at 25 °C, and then diluted with H₂O 40 mL and then extracted with EtOAc (100 mL*3). The combined organic layers were washed with brine (50 mL*3), dried over Na₂SO₄, filtered and concentrated under reduced pressure. The residue was purified by column chromatography (SiO₂, Petroleum ether:Ethyl acetate=5:1 to 1:1) to give the title compound (1.70 g, 3.47 mmol, 33.7% yield, 92.8% purity) as a light yellow solid. LCMS (ES+): m/z 456.3 [M+H]+.

Synthesis of 5-fluoro-4-(3-(piperidin-1-yl)phenyl)-N-(piperidin-4-yl)pyrimidin-2-amine. To a solution of tert-butyl 4-[[5-fluoro-4-[3-(1-piperidyl)phenyl]pyrimidin-2-yl]amino]piperidine-1-carboxylate (700 mg, 1.54 mmol, 1.00 eq) was added HCl/EtOAc (4.00 M, 10.0 mL, 26.0 eq) at 25 °C. The mixture was stirred at 25 °C for 1 h. LC-MS showed the desired mass was detected. The reaction mixture was concentrated under reduced pressure to give the title compound (600 mg, crude, HCl) as a white solid. LCMS (ES+): m/z 356.3 [M+H]+.

Synthesis of trans-3-((3-fluoro-4-(1-((4-(4-((5-fluoro-4-(3-(piperidin-1-yl)phenyl)pyrimidin-2-yl)amino)piperidine-1-carbonyl)cyclohexyl)methyl)piperidin-4-yl)phenyl)amino)piperidine-2,6-dione (Compound 682). To a solution of 5-fluoro-N-(4-piperidyl)-4-[3-(1-piperidyl)phenyl]pyrimidin-2-amine (100 mg, 255 μmol, 1.00 eq, HCl) and trans-4-[[4-[4-[(2,6-dioxo-3-piperidyl)amino]-2-fluoro-phenyl]-1-piperidyl]methyl]cyclohexanecarboxylic acid (113 mg, 203 μmol, 0.796 eq, TFA) in DMF (1 mL) was added DIEA (98.9 mg, 765 μmol, 133 μL, 3.00 eq) at 25 °C. The mixture was stirred at 25 °C for 1 h, then the HATU (97.0 mg, 255 μmol, 1.00 eq) was added to the solution at 25 °C, The mixture was stirred at 25 °C for 11 h. LC-MS showed the desired mass was detected. The reaction mixture was concentrated under reduced pressure. The residue was purified by prep-HPLC (column: Phenomenex Luna C18 75*30 mm*3 um; mobile phase: [water (0.04% HCl)-ACN]; gradient: 15%-45% B over 8.0 min) to give desired compound as a pale yellow solid. The residue was purified by prep-HPLC (column: Phenomenex Luna C18 75*30 mm*3 um; mobile phase: [H₂O (0.2% FA)-ACN]; gradient: 10%-50% B over 8.0 min) to give the title compound (Compound 682) (13.1 mg, 15.9 μmol, 6.25% yield, 98.1% purity, 0.500 FA) as a pale yellow solid. LCMS (ES+): m/z 783.5 [M+H]+.

### Example 643. Synthesis of trans-4-((5-fluoro-4-(3-(piperidin-1-yl)phenyl)pyrimidin-2-yl)amino)cyclohexyl 4-(4-((2,6-dioxopiperidin-3-yl)amino)-2-fluorophenyl)-[1,4′-bipiperidine]-1′-carboxylate (Compound 689)

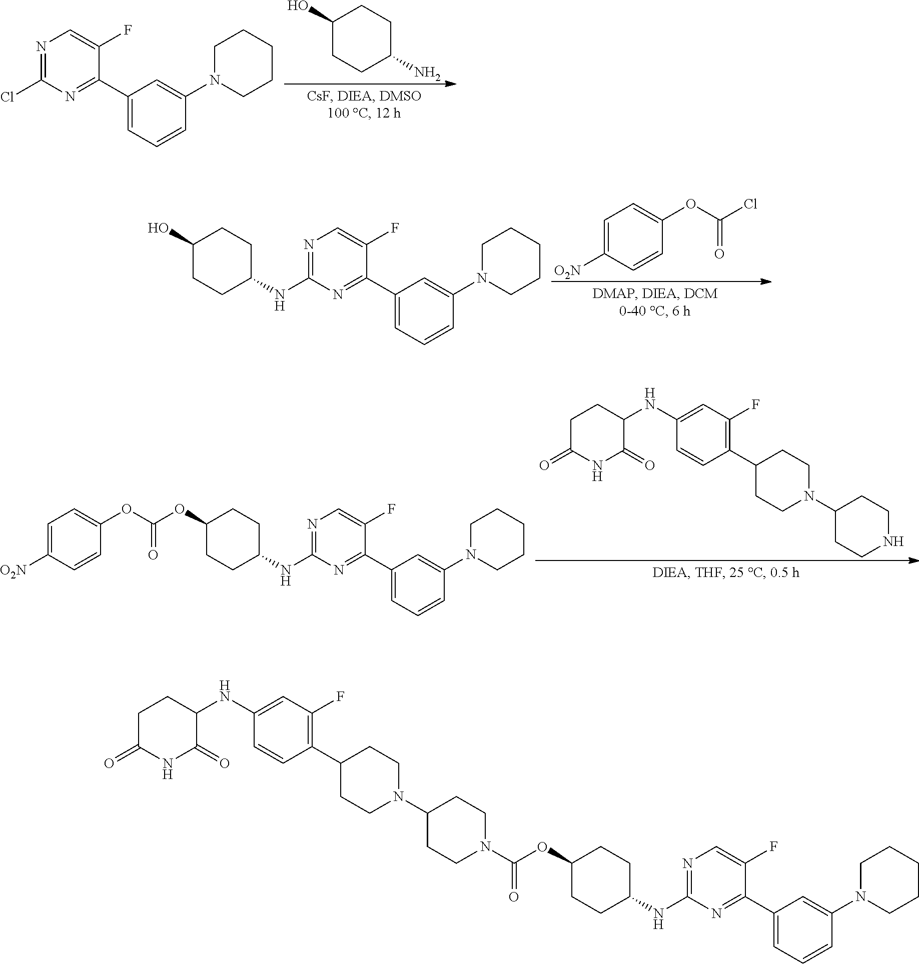

Synthesis of trans-4-((5-fluoro-4-(3-(piperidin-1-yl)phenyl)pyrimidin-2-yl)amino)cyclohexan-1-ol. To a solution of 2-chloro-5-fluoro-4-(3-(piperidin-1-yl)phenyl)pyrimidine (3.00 g, 10.3 mmol, 1.00 eq) in DMSO (100 mL) were added trans-4-aminocyclohexan-1-ol (1.78 g, 15.4 mmol, 1.50 eq), CsF (2.34 g, 15. mmol, 1.50 eq) and DIEA (3.99 g, 30.9 mmol, 5.37 mL, 3.00 eq) at 25 °C. The mixture was stirred at 100 °C for 12 h. LC-MS showed 2-chloro-5-fluoro-4-(3-(piperidin-1-yl)phenyl)pyrimidine was consumed completely and the desired mass was detected. Water (100 mL) was added to the mixture at 0 °C. The mixture was filtered and the filter cake was dried under reduced pressure to give the title compound (5.00 g, crude) as a yellow solid. LCMS (ES⁺): m/z 371.2 [M+H]⁺

Synthesis of trans-4-((5-fluoro-4-(3-(piperidin-1-yl)phenyl)pyrimidin-2-yl)amino)cyclohexyl (4-nitrophenyl) carbonate. To a solution of trans-4-((5-fluoro-4-(3-(piperidin-1-yl)phenyl)pyrimidin-2-yl)amino)cyclohexan-1-ol (3.00 g, 8.10 mmol, 1.00 eq) in DCM (150 mL) were added 4-nitrophenyl carbonochloridate (8.16 g, 40.5 mmol, 5.00 eq), DMAP (1.48 g, 12.2 mmol, 1.50 eq) and DIEA (3.14 g, 24.3 mmol, 4.23 mL, 3.00 eq) at 0 °C. The mixture was stirred at 25 °C for 3 h. LC-MS showed trans-4-((5-fluoro-4-(3-(piperidin-1-yl)phenyl)pyrimidin-2-yl)amino)cyclohexan-1-ol was remaining, and the desired mass was detected. Then the mixture was stirred at 40 °C for 3 h. LC-MS showed trans-4-((5-fluoro-4-(3-(piperidin-1-yl)phenyl)pyrimidin-2-yl)amino)cyclohexan-1-ol was consumed completely and the desired mass was detected. The reaction mixture was concentrated under reduced pressure to give a residue. EtOAc (50 mL) was added to the residue at 25 °C. The mixture was filtered and the filter cake was dried under reduced pressure to give the title compound (3.00 g, crude) as a yellow solid. LCMS (ES⁺): m/z 536.2 [M+H]⁺

Synthesis of trans-4-((5-fluoro-4-(3-(piperidin-1-yl)phenyl)pyrimidin-2-yl)amino)cyclohexyl 4-(4-((2,6-dioxopiperidin-3-yl)amino)-2-fluorophenyl)-[1,4′-bipiperidine]-1′-carboxylate (Compound 689). To a solution of 3-((4-([1,4′-bipiperidin]-4-yl)-3-fluorophenyl)amino)piperidine-2,6-dione (205 mg, 408 μmol, 1.00 eq, TFA) in THF (7 mL) were added DIEA (158 mg, 1.22 mmol, 213 μL, 3.00 eq) and trans-4-((5-fluoro-4-(3-(piperidin-1-yl)phenyl)pyrimidin-2-yl)amino)cyclohexyl (4-nitrophenyl) carbonate (131 mg, 245 μmol, 0.600 eq) at 25 °C. The mixture was stirred at 25 °C for 0.5 h. LC-MS showed trans-4-((5-fluoro-4-(3-(piperidin-1-yl)phenyl)pyrimidin-2-yl)amino)cyclohexyl (4-nitrophenyl) carbonate was consumed completely and the desired mass was detected. The reaction mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 10%-40% B over 8.0 min) to give the title compound (Compound 689) (54.7 mg, 68.6 μmol, 16.8% yield, 98.5% purity) as a white solid. LCMS (ES⁺): m/z 785.5 [M+H]⁺.

### Example 644. Synthesis of trans-4-((5-fluoro-4-(3-(piperidin-1-yl)phenyl)pyrimidin-2-yl)amino)cyclohexyl 4-(4-(4-((2,6-dioxopiperidin-3-yl)amino)-2-fluorophenyl)piperazin-1-yl)piperidine-1-carboxylate (Compound 687)

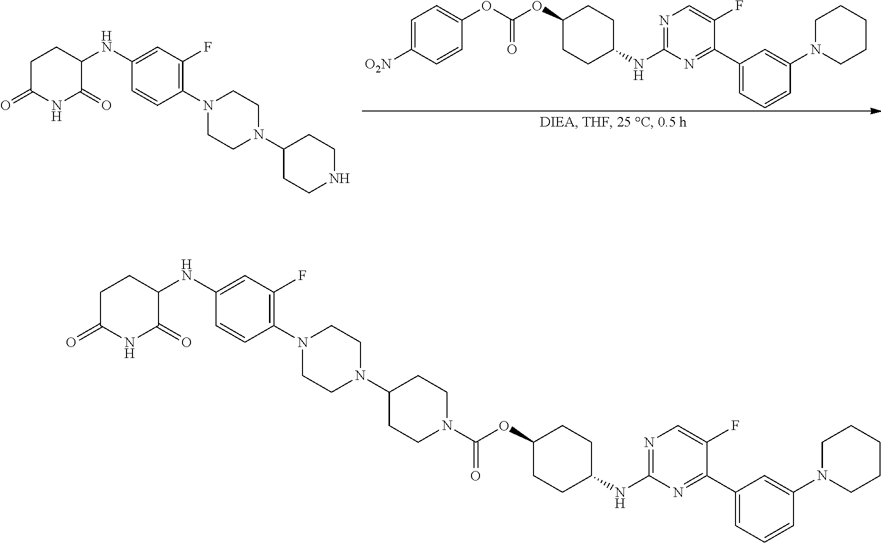 [SMILES: O=C1CCC(Nc2ccc(N3CCN(C4CCNCC4)CC3)c(F)c2)C(=O)N1 ; O=C(Oc1ccc([N+](=O)[O-])cc1)O[C@H]1CC[C@H](Nc2ncc(F)c(-c3cccc(N4CCCCC4)c3)n2)CC1 ; O=C1CCC(Nc2ccc(N3CCN(C4CCN(C(=O)O[C@H]5CC[C@H](Nc6ncc(F)c(-c7cccc(N8CCCCC8)c7)n6)CC5)CC4)CC3)c(F)c2)C(=O)N1]

Synthesis of trans-4-((5-fluoro-4-(3-(piperidin-1-yl)phenyl)pyrimidin-2-yl)amino)cyclohexyl 4-(4-(4-((2,6-dioxopiperidin-3-yl)amino)-2-fluorophenyl)piperazin-1-yl)piperidine-1-carboxylate (Compound 687). To a stirred solution of 3-[3-fluoro-4-[4-(4-piperidyl)piperazin-1-yl]anilino]piperidine-2,6-dione (200 mg, 397 μmol, 1.00 eq, TFA) and DIEA (154 mg, 1.19 mmol, 207 μL, 3.00 eq) in THF (4 mL) was added trans-4-((5-fluoro-4-(3-(piperidin-1-yl)phenyl)pyrimidin-2-yl)amino)cyclohexyl (4-nitrophenyl) carbonate (127 mg, 237 μmol, 0.600 eq) at 25 °C. The resulting mixture was stirred at 25 °C for 0.5 h. LCMS showed 3-[3-fluoro-4-[4-(4-piperidyl)piperazin-1-yl]anilino]piperidine-2,6-dione was consumed completely and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 50%-85% B over 8.0 min) to give the title compound (Compound 687) (56.5 mg, 71.6 μmol, 13.9% yield, 99.6% purity) as a pale yellow solid. LCMS (ES⁺): m/z 786.4 [M+H]⁺.

### Example 645. Synthesis of trans-(4-((4-(4-((2,6-dioxopiperidin-3-yl)amino)-2-fluorophenyl)piperidin-1-yl)methyl)cyclohexyl)methyl 4-((4-([1,1′-biphenyl]-3-yl)-5-fluoropyrimidin-2-yl)amino)piperidine-1-carboxylate (Compound 640)

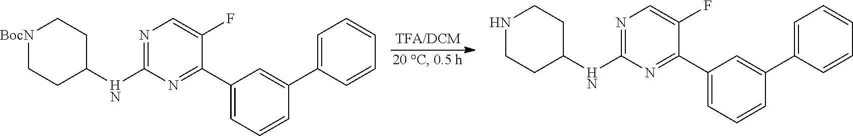 [SMILES: CC(C)(C)OC(=O)N1CCC(Nc2ncc(F)c(-c3cccc(-c4ccccc4)c3)n2)CC1 ; Fc1cnc(NC2CCNCC2)nc1-c1cccc(-c2ccccc2)c1]

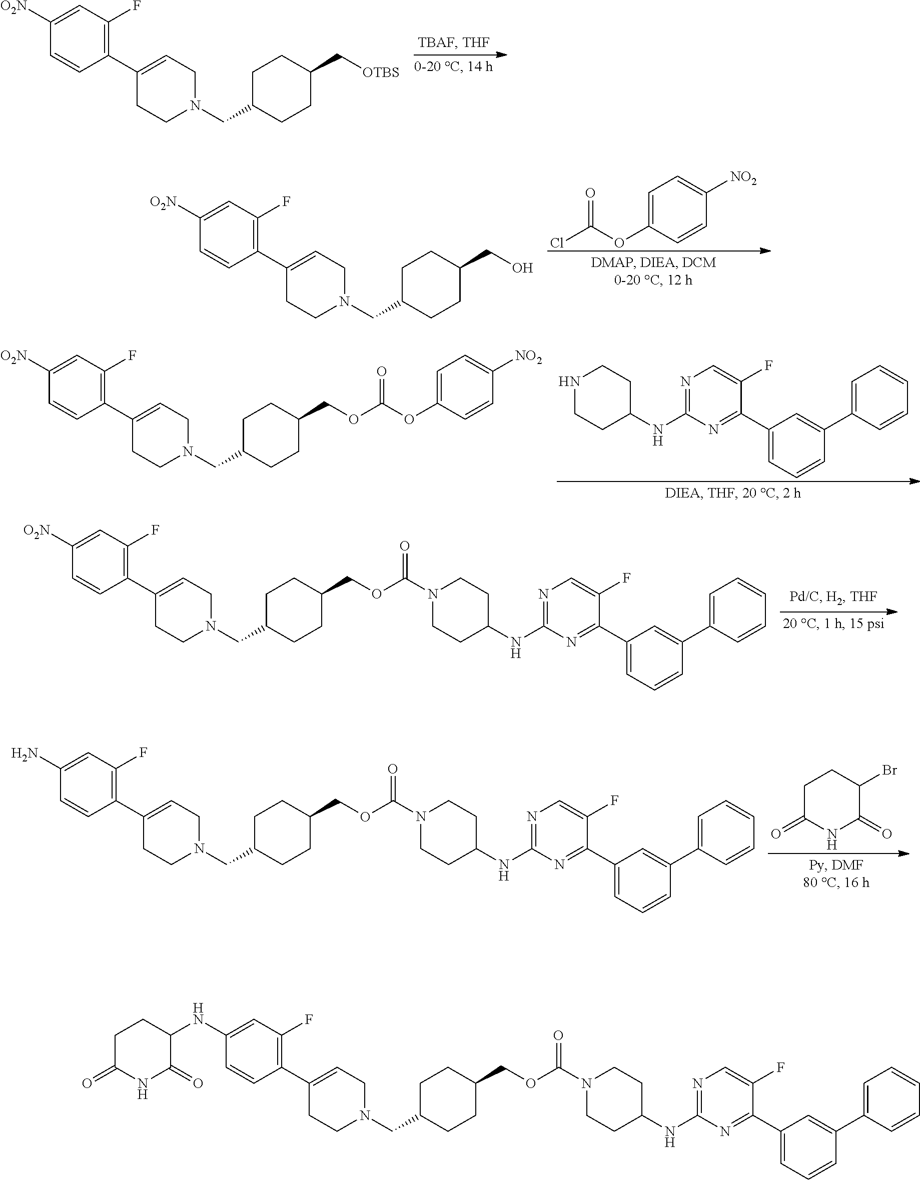

Synthesis of 4-([1,1′-biphenyl]-3-yl)-5-fluoro-N-(piperidin-4-yl)pyrimidin-2-amine. A solution of tert-butyl 4-((4-([1,1′-biphenyl]-3-yl)-5-fluoropyrimidin-2-yl)amino)piperidine-1-carboxylate (160 mg, 357 μmol, 1.00 eq) in DCM (1.5 mL) and TFA (0.5 mL) was stirred at 20 °C for 0.5 h. LCMS showed tert-butyl 4-((4-([1,1′-biphenyl]-3-yl)-5-fluoropyrimidin-2-yl)amino)piperidine-1-carboxylate was consumed completely and the desired mass was detected. The reaction mixture was concentrated under reduced pressure to give the title compound (164 mg, crude, TFA) as a colorless oil. LCMS (ES⁺): m/z 349.2 [M+H]⁺

Synthesis of trans-(4-((4-(2-fluoro-4-nitrophenyl)-3,6-dihydropyridin-1 (2H)-yl)methyl)cyclohexyl)methanol. To a stirred solution of trans-1-((4-(((tert-butyldimethylsilyl)oxy)methyl)cyclohexyl)methyl)-4-(2-fluoro-4-nitrophenyl)-1,2,3,6-tetrahydropyridine (500 mg, 1.08 mmol, 1.00 eq) in THF (10 mL) was added TBAF (1.00 M, 2.16 mL, 2.00 eq) at 0 °C. The resulting mixture was stirred at 20 °C for 14 h. LCMS showed trans-1-((4-(((tert-butyldimethylsilyl)oxy)methyl)cyclohexyl)methyl)-4-(2-fluoro-4-nitrophenyl)-1,2,3,6-tetrahydropyridine was consumed completely and the desired mass was detected. H₂O (10 mL) was added to the mixture and the mixture was extracted with EtOAc (15 mL*3). The combined organic layers were dried over Na₂SO₄, filtered and the filtrate was concentrated under reduced pressure to a residue. The residue was purified by prep-TLC (SiO₂, PE/EtOAc=0/1) to give the title compound (210 mg, 603 μmol, 55.8% yield) as a yellow solid. LCMS (ES⁺): m/z 349.2 [M+H]⁺

Synthesis of trans-(4-((4-(2-fluoro-4-nitrophenyl)-3,6-dihydropyridin-1 (2H)-yl)methyl)cyclohexyl)methyl (4-nitrophenyl) carbonate. To a solution of trans-(4-((4-(2-fluoro-4-nitrophenyl)-3,6-dihydropyridin-1 (2H)-yl)methyl)cyclohexyl)methanol (210 mg, 603 μmol, 1.00 eq) and 4-nitrophenyl carbonochloridate (243 mg, 1.21 mmol, 2.00 eq) in DCM (5 mL) were added DMAP (73.6 mg, 603 μmol, 1.00 eq) and DIEA (390 mg, 3.02 mmol, 525 μL, 5.00 eq) at 0 °C. The mixture was slowly warmed to 20 °C and stirred at 20 °C for 12 h. LCMS showed trans-(4-((4-(2-fluoro-4-nitrophenyl)-3,6-dihydropyridin-1 (2H)-yl)methyl)cyclohexyl)methanol was consumed and the desired mass was detected. To the mixture was added H₂O (5 mL) and the mixture was extracted with DCM (5 mL*3). The combined organic layers were dried over Na₂SO₄, filtered and the filtrate was concentrated under reduced pressure to give a residue. The residue was purified by prep-TLC (SiO₂, PE/EtOAc=2/1) to give the title compound (130 mg, 253 μmol, 42.0% yield) as a brown solid. LCMS (ES⁺): m/z 514.2 [M+H]⁺

Synthesis of trans-(4-((4-(2-fluoro-4-nitrophenyl)-3,6-dihydropyridin-1 (2H)-yl)methyl)cyclohexyl)methyl 4-((4-([1,1′-biphenyl]-3-yl)-5-fluoropyrimidin-2-yl)amino)piperidine-1-carboxylate. To a solution of trans-(4-((4-(2-fluoro-4-nitrophenyl)-3,6-dihydropyridin-1 (2H)-yl)methyl)cyclohexyl)methyl (4-nitrophenyl) carbonate (124 mg, 242 mol, 0.700 eq) and 4-([1,1′-biphenyl]-3-yl)-5-fluoro-N-(piperidin-4-yl)pyrimidin-2-amine (160 mg, 346 μmol, 1.00 eq, TFA) in THE (5 mL) was added DIEA (224 mg, 1.73 mmol, 303 μL, 5.00 eq) at 20 °C, then the mixture was stirred at 20 °C for 2 h. LCMS showed trans-(4-((4-(2-fluoro-4-nitrophenyl)-3,6-dihydropyridin-1 (2H)-yl)methyl)cyclohexyl)methyl (4-nitrophenyl) carbonate was consumed and the desired mass was detected. To the mixture was added H₂O (5 mL) and the mixture was extracted with EtOAc (5 mL*3). The combined organic layers were dried over Na₂SO₄, filtered and the filtrate was concentrated under reduced pressure to give a residue. The residue was purified by prep-TLC (SiO₂, PE/EtOAc=0/1) to give the title compound (160 mg, 221 μmol, 64.0% yield) as a brown oil. LCMS (ES⁺): m/z 723.4 [M+H]⁺

Synthesis of trans-(4-((4-(4-amino-2-fluorophenyl)piperidin-1-yl)methyl)cyclohexyl)methyl 4-((4-([1,1′-biphenyl]-3-yl)-5-fluoropyrimidin-2-yl)amino)piperidine-1-carboxylate. 10% Pd/C (160 mg) was added to the reaction flask at 20 °C under N₂ atmosphere, and then THE (5 mL) was added to the reaction flask under N₂, then trans-(4-((4-(2-fluoro-4-nitrophenyl)-3,6-dihydropyridin-1 (2H)-yl)methyl)cyclohexyl)methyl 4-((4-([1,1′-biphenyl]-3-yl)-5-fluoropyrimidin-2-yl)amino)piperidine-1-carboxylate (160 mg, 221 μmol, 1.00 eq) was added to the mixture at 20 °C under N₂ and the mixture was degassed and purged with H₂ for three times. The mixture was stirred at 20 °C for 1 h under H₂ (15 psi) atmosphere. LCMS showed trans-(4-((4-(2-fluoro-4-nitrophenyl)-3,6-dihydropyridin-1 (2H)-yl)methyl)cyclohexyl)methyl 4-((4-([1,1′-biphenyl]-3-yl)-5-fluoropyrimidin-2-yl)amino)piperidine-1-carboxylate was consumed and the desired mass was detected. The mixture was filtered and the filtrate was concentrated under reduced pressure to give the title compound (150 mg, crude) as a colorless oil. LCMS (ES⁺): m/z 695.5 [M+H]⁺

Synthesis of trans-(4-((4-(4-((2,6-dioxopiperidin-3-yl)amino)-2-fluorophenyl)piperidin-1-yl)methyl)cyclohexyl)methyl 4-((4-([1,1′-biphenyl]-3-yl)-5-fluoropyrimidin-2-yl)amino)piperidine-1-carboxylate (Compound 640). To a stirred solution of trans-(4-((4-(4-amino-2-fluorophenyl)piperidin-1-yl)methyl)cyclohexyl)methyl 4-((4-([1,1′-biphenyl]-3-yl)-5-fluoropyrimidin-2-yl)amino)piperidine-1-carboxylate (60.0 mg, 86.4 mol, 1.00 eq) and 3-bromopiperidine-2,6-dione (49.7 mg, 259 μmol, 3.00 eq) in DMF (3 mL) was added Py (13.7 mg, 173 μmol, 13.9 μL, 2.00 eq) at 20 °C. The resulting mixture was stirred at 80 °C for 2 h. LCMS showed trans-(4-((4-(4-amino-2-fluorophenyl)piperidin-1-yl)methyl)cyclohexyl)methyl 4-((4-([1,1′-biphenyl]-3-yl)-5-fluoropyrimidin-2-yl)amino)piperidine-1-carboxylate remained and the desired mass was detected, then 3-bromopiperidine-2,6-dione (33.2 mg, 173 μmol, 2.00 eq) was added to the mixture at 20 °C, the mixture was stirred at 80 °C for 12 h. LCMS showed trans-(4-((4-(4-amino-2-fluorophenyl)piperidin-1-yl)methyl)cyclohexyl)methyl 4-((4-([1,1′-biphenyl]-3-yl)-5-fluoropyrimidin-2-yl)amino)piperidine-1-carboxylate remained and the desired mass was detected. Then 3-bromopiperidine-2,6-dione (33.2 mg, 173 μmol, 2.00 eq) and Py (13.7 mg, 173 μmol, 13.9 μL, 2.00 eq) were added to the mixture at 20 °C, the mixture was stirred at 80 °C for 2 h. LCMS showed trans-(4-((4-(4-amino-2-fluorophenyl)piperidin-1-yl)methyl)cyclohexyl)methyl 4-((4-([1,1′-biphenyl]-3-yl)-5-fluoropyrimidin-2-yl)amino)piperidine-1-carboxylate was consumed completely and the desired mass was detected. The mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 40%-75% B over 8.0 min) to give the title compound (Compound 640) (5.40 mg, 6.61 μmol, 7.66% yield, 98.7% purity) as a pale yellow solid. LCMS (ES⁺): m/z 806.5 [M+H]⁺

### Example 646. Synthesis of trans-4-((4-([1,1′-biphenyl]-3-yl)-5-fluoropyrimidin-2-yl)amino)cyclohexyl 4-(4-((2,6-dioxopiperidin-3-yl)amino)-2-fluorophenyl)-[1,4′-bipiperidine]-1′-carboxylate (Compound 651)

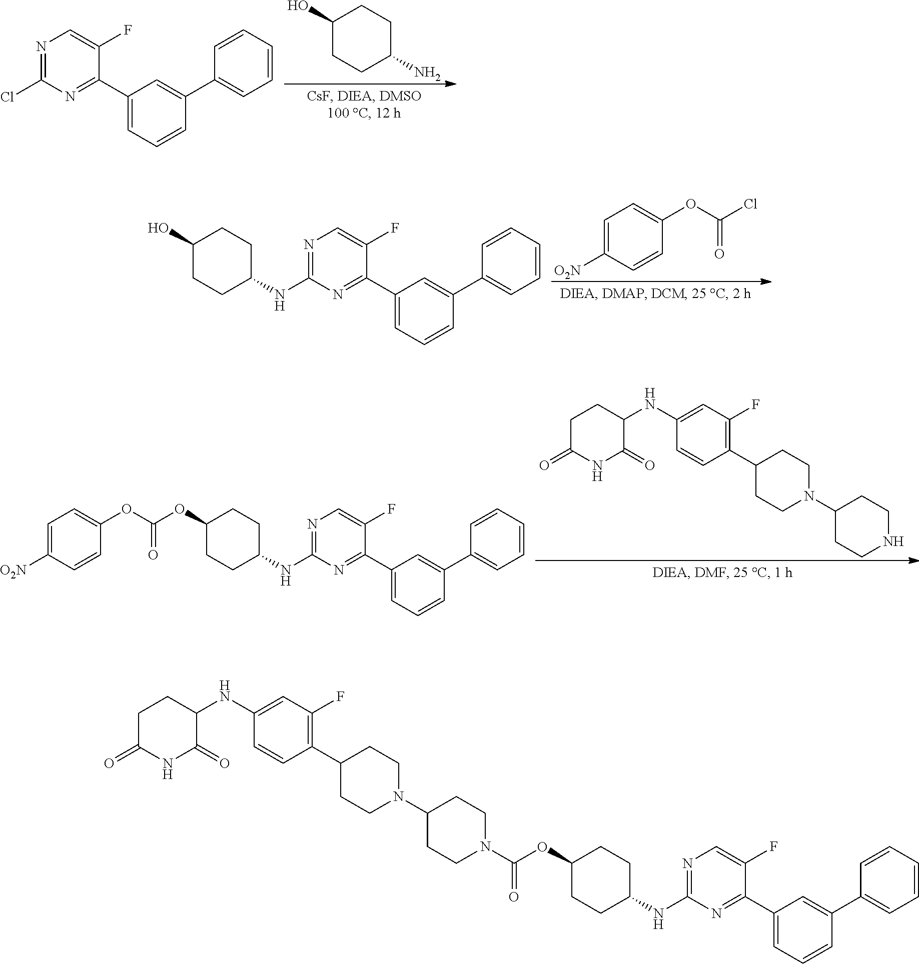

Synthesis of trans-4-((4-([1,1′-biphenyl]-3-yl)-5-fluoropyrimidin-2-yl)amino)cyclohexan-1-ol. To a solution of 4-([1,1′-biphenyl]-3-yl)-2-chloro-5-fluoropyrimidine (5.00 g, 17.5 mmol, 1.00 eq) in DMSO (50 mL) was added trans-4-aminocyclohexan-1-ol (2.22 g, 19.3 mmol, 1.10 eq), CsF (5.34 g, 35.1 mmol, 2.00 eq) and DIEA (4.54 g, 35.1 mmol, 6.12 mL, 2.00 eq) at 25 °C. The mixture was stirred at 100 °C for 12 h. LC-MS showed the desired mass was detected. The solid was filtered, washed with PE (3*20 mL), and dried under high vacuum to give the title compound (9.00 g, crude) as a yellow solid. LCMS (ES⁺): m/z 364.1 [M+H]⁺.

Synthesis of trans-4-((4-([1,1′-biphenyl]-3-yl)-5-fluoropyrimidin-2-yl)amino)cyclohexyl (4-nitrophenyl) carbonate. To a solution of 4-[[5-fluoro-4-(3-phenylphenyl)pyrimidin-2-yl]amino]cyclohexanoltrans-4-((4-([1,1′-biphenyl]-3-yl)-5-fluoropyrimidin-2-yl)amino)cyclohexan-1-ol (3.00 g, 8.25 mmol, 1.00 eq) in DCM (30 mL) was added 4-nitrophenyl carbonochloridate (3.16 g, 15.6 mmol, 1.90 eq), DIEA (4.05 g, 31.3 mmol, 5.46 mL, 3.80 eq) and DMAP (907 mg, 7.43 mmol, 0.900 eq) at 25 °C. The mixture was stirred at 25 °C for 2 h. LC-MS showed the desired mass was detected. The reaction mixture was concentrated under high vacuum. The residue was purified by column chromatography (SiO₂, Petroleum ether:Ethyl acetate=1:0 to 3:1) to give the title compound (2.00 g, 3.27 mmol, 39.5% yield, 86.3% purity) as a yellow solid. LCMS (ES⁺): m/z 529.3 [M+H]⁺.

Synthesis of trans-4-((4-([1,1′-biphenyl]-3-yl)-5-fluoropyrimidin-2-yl)amino)cyclohexyl 4-(4-((2,6-dioxopiperidin-3-yl)amino)-2-fluorophenyl)-[1,4′-bipiperidine]-1′-carboxylate (Compound 651). To a solution of 3-((4-([1,4′-bipiperidin]-4-yl)-3-fluorophenyl)amino)piperidine-2,6-dione (180 mg, 358 μmol, 1.00 eq, TFA) in DMF (2 mL) was added DIEA (46.2 mg, 358 μmol, 62.3 μL, 1.00 eq) and trans-4-((4-([1,1′-biphenyl]-3-yl)-5-fluoropyrimidin-2-yl)amino)cyclohexyl (4-nitrophenyl) carbonate (94.6 mg, 179 μmol, 0.500 eq) at 25 °C. The mixture was stirred at 25 °C for 1 h. LC-MS showed the desired mass was detected. The reaction mixture was filtered and the filtrate concentrated under high vacuum. The residue was purified by prep-HPLC (Waters Xbridge Prep OBD C18 150*40 mm*10 um; mobile phase: [H₂O (10 mM NH₄HCO₃)-ACN]; gradient: 60%-95% B over 8.0 min) to give the title compound (Compound 651) (37.8 mg, 46.7 μmol, 13.0% yield, 96.2% purity) as a white solid. LCMS (ES⁺): m/z 778.4 [M+H]⁺.

### Example 647. Synthesis of trans-[4-[[5-fluoro-4-(3-phenylphenyl)pyrimidin-2-yl]amino]cyclohexyl]4-[4-[4-[(2,6-dioxo-3-piperidyl)amino]-2-fluoro-phenyl]piperazin-1-yl]piperidine-1-carboxylate (Compound 664)

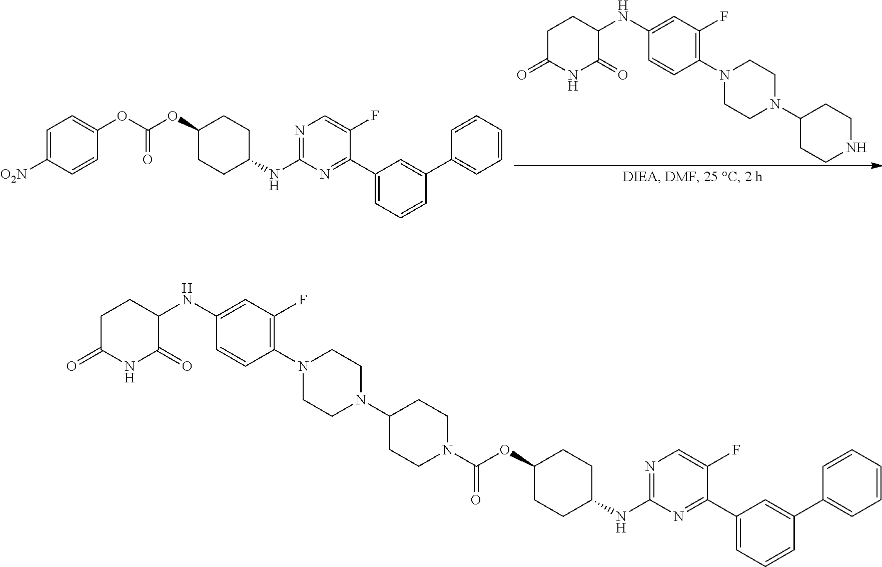 [SMILES: O=C(Oc1ccc([N+](=O)[O-])cc1)O[C@H]1CC[C@H](Nc2ncc(F)c(-c3cccc(-c4ccccc4)c3)n2)CC1 ; O=C1CCC(Nc2ccc(N3CCN(C4CCNCC4)CC3)c(F)c2)C(=O)N1 ; O=C1CCC(Nc2ccc(N3CCN(C4CCN(C(=O)O[C@H]5CC[C@H](Nc6ncc(F)c(-c7cccc(-c8ccccc8)c7)n6)CC5)CC4)CC3)c(F)c2)C(=O)N1]

Synthesis of trans-[4-[[5-fluoro-4-(3-phenylphenyl)pyrimidin-2-yl]amino]cyclohexyl]4-[4-[4-[(2,6-dioxo-3-piperidyl)amino]-2-fluoro-phenyl]piperazin-1-yl]piperidine-1-carboxylate (Compound 664). To a solution of trans-[4-[[5-fluoro-4-(3-phenylphenyl)pyrimidin-2-yl]amino]cyclohexyl](4-nitrophenyl) carbonate (100 mg, 189 μmol, 1.00 eq) in DMF (2 mL) was added DIEA (244 mg, 1.89 mmol, 329 μL, 10.0 eq) and 3-[3-fluoro-4-[4-(4-piperidyl)piperazin-1-yl]anilino]piperidine-2,6-dione (73.69 mg, 146.36 mol, 0.774 eq, TFA) at 25 °C, The mixture was stirred at 25 °C for 2 h. LC-MS showed trans-[4-[[5-fluoro-4-(3-phenylphenyl)pyrimidin-2-yl]amino]cyclohexyl](4-nitrophenyl) carbonate was consumed completely and the desired mass was detected. The reaction mixture was concentrated under reduced pressure. The residue was purified by prep-HPLC (column: Phenomenex Luna C18 100*30 mm*3 um; mobile phase: [H₂O (0.2% FA)-ACN]; gradient: 30%-60% B over 8.0 min) to give the title compound (Compound 664) (52.1 mg, 65.55 μmol, 34.65% yield, 98.00% purity) was obtained as a pale yellow solid. LCMS (ES⁺): m/z 779.4 [M+H]⁺.

### Example 648. Synthesis of trans-4-((4-([1,1′-biphenyl]-3-yl)-5-fluoropyrimidin-2-yl)amino)cyclohexyl 4-((4-(3-(2,6-dioxopiperidin-3-yl)-1-methyl-1H-indazol-6-yl)piperidin-1-yl)methyl)piperidine-1-carboxylate (Compound 647)

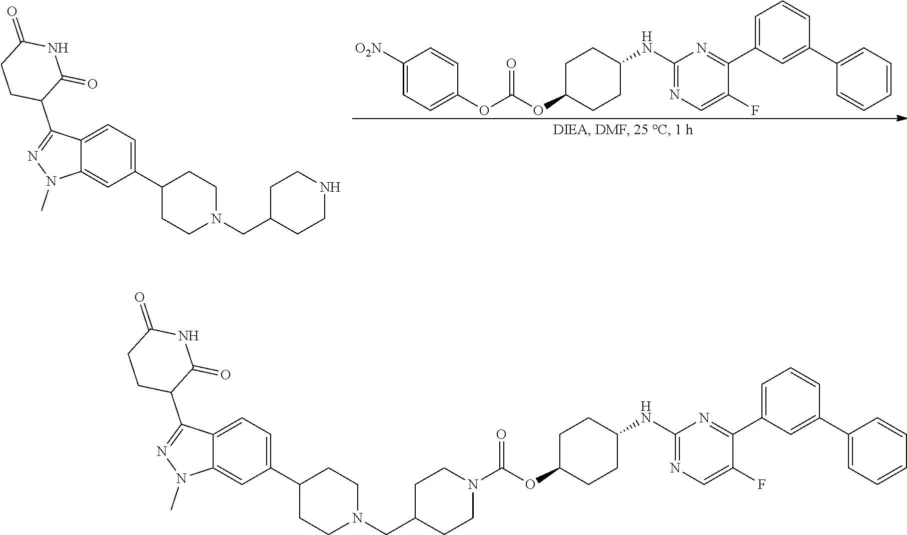 [SMILES: Cn1nc(C2CCC(=O)NC2=O)c2ccc(C3CCN(CC4CCNCC4)CC3)cc21 ; O=C(Oc1ccc([N+](=O)[O-])cc1)O[C@H]1CC[C@H](Nc2ncc(F)c(-c3cccc(-c4ccccc4)c3)n2)CC1 ; Cn1nc(C2CCC(=O)NC2=O)c2ccc(C3CCN(CC4CCN(C(=O)O[C@H]5CC[C@H](Nc6ncc(F)c(-c7cccc(-c8ccccc8)c7)n6)CC5)CC4)CC3)cc21]

Synthesis of trans-4-((4-([1,1′-biphenyl]-3-yl)-5-fluoropyrimidin-2-yl)amino)cyclohexyl 4-((4-(3-(2,6-dioxopiperidin-3-yl)-1-methyl-1H-indazol-6-yl)piperidin-1-yl)methyl)piperidine-1-carboxylate (Compound 647). To a solution of 3-(1-methyl-6-(1-(piperidin-4-ylmethyl)piperidin-4-yl)-1H-indazol-3-yl)piperidine-2,6-dione (180 mg, 334 μmol, 1.00 eq, TFA) in DMF (3 mL) was added DIEA (86.5 mg, 669 μmol, 116 μL, 2.00 eq) and [4-[[5-fluoro-4-(3-phenylphenyl)pyrimidin-2-yl]amino]cyclohexyl](4-nitrophenyl) carbonate (88.4 mg, 167 μmol, 0.500 eq) at 25 °C. The mixture was stirred at 25 °C for 1 h. LC-MS showed the desired mass was detected. The reaction mixture was filtered and the filtrate concentrated under high vacuum. The residue was purified by prep-HPLC (Phenomenex Luna C18 100*30 mm*3 um; mobile phase: [H₂O (0.2% FA)-ACN]; gradient: 30%-70% B over 8.0 min) to give the title compound (Compound 647) (108 mg, 131 μmol, 39.1% yield, 100% purity, 0.3 FA) as a pale yellow solid. LCMS (ES⁺): m/z 813.5 [M+H]⁺.

### Example 649. Synthesis of trans-4-((4-([1,1′-biphenyl]-3-yl)-5-fluoropyrimidin-2-yl)amino)cyclohexyl 4-((4-(3-(2,6-dioxopiperidin-3-yl)-1-methyl-1H-indazol-6-yl)piperidin-1-yl)methyl)-4-hydroxypiperidine-1-carboxylate (Compound 646)

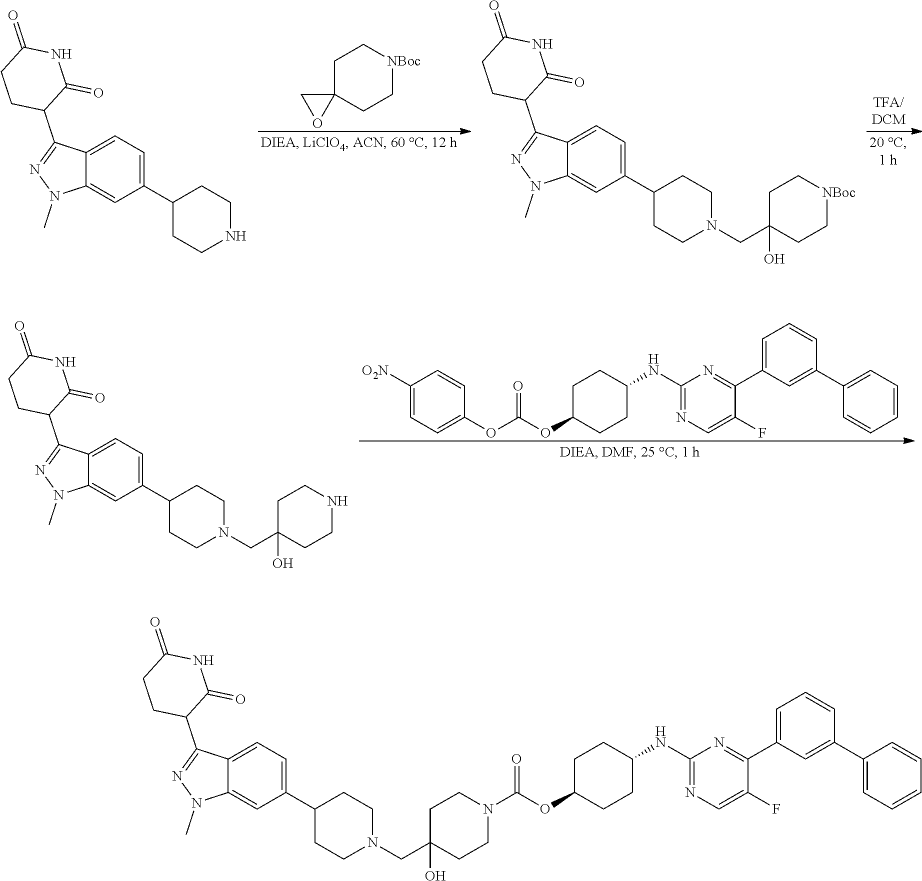 [SMILES: Cn1nc(C2CCC(=O)NC2=O)c2ccc(C3CCNCC3)cc21 ; CC(C)(C)OC(=O)N1CCC2(CC1)CO2 ; Cn1nc(C2CCC(=O)NC2=O)c2ccc(C3CCN(CC4(O)CCN(C(=O)OC(C)(C)C)CC4)CC3)cc21 ; Cn1nc(C2CCC(=O)NC2=O)c2ccc(C3CCN(CC4(O)CCNCC4)CC3)cc21 ; O=C(Oc1ccc([N+](=O)[O-])cc1)O[C@H]1CC[C@H](Nc2ncc(F)c(-c3cccc(-c4ccccc4)c3)n2)CC1 ; Cn1nc(C2CCC(=O)NC2=O)c2ccc(C3CCN(CC4(O)CCN(C(=O)O[C@H]5CC[C@H](Nc6ncc(F)c(-c7cccc(-c8ccccc8)c7)n6)CC5)CC4)CC3)cc21]

Synthesis of tert-butyl 4-((4-(3-(2,6-dioxopiperidin-3-yl)-1-methyl-1H-indazol-6-yl)piperidin-1-yl)methyl)-4-hydroxypiperidine-1-carboxylate. To a solution of 3-(1-methyl-6-(piperidin-4-yl)-1H-indazol-3-yl)piperidine-2,6-dione (1.00 g, 2.27 mmol, 1.00 eq, TFA) in ACN (20 mL) was added DIEA (586 mg, 4.54 mmol, 790 μL, 2.00 eq), tert-butyl 1-oxa-6-azaspiro[2.5]octane-6-carboxylate (968 mg, 4.54 mmol, 2.00 eq) and LiClO₄ (340 mg, 3.20 mmol, 140 μL, 1.41 eq) at 25 °C. The mixture was stirred at 60 °C for 12 h. LC-MS showed the desired mass was detected. The reaction mixture was quenched by addition Na₂SO₃ aq. 10 mL at 25 °C, and then diluted with H₂O 50 mL and then extracted with EtOAc (10 mL*3). The combined organic layers were washed with brine (10 mL*3), dried over Na₂SO₄, filtered and concentrated under reduced pressure to give a residue. The residue was purified by column chromatography (SiO₂, PE:THF=1:0 to 0:1) to give the title compound (2.00 g, crude) as a yellow solid. LCMS (ES⁺): m/z 540.4 [M+H]⁺.

Synthesis of 3-(6-(1-((4-hydroxypiperidin-4-yl)methyl)piperidin-4-yl)-1-methyl-1H-indazol-3-yl)piperidine-2,6-dione. To a solution of tert-butyl 4-((4-(3-(2,6-dioxopiperidin-3-yl)-1-methyl-1H-indazol-6-yl)piperidin-1-yl)methyl)-4-hydroxypiperidine-1-carboxylate (500 mg, 926 μmol, 1.00 eq) in DCM (5 mL) was added TFA (3.07 g, 26.9 mmol, 2 mL, 29.0 eq) at 20 °C. The mixture was stirred at 20 °C for 1 h. TLC (PE:THF=0:1, Rf=0.07) indicated tert-butyl 4-((4-(3-(2,6-dioxopiperidin-3-yl)-1-methyl-1H-indazol-6-yl)piperidin-1-yl)methyl)-4-hydroxypiperidine-1-carboxylate was consumed completely and one main new spot formed. The reaction mixture was concentrated under high vacuum to give the title compound (400 mg, crude, TFA) as a green oil. LCMS (ES⁺): m/z 440.3 [M+H]⁺.

Synthesis of trans-4-((4-([1,1′-biphenyl]-3-yl)-5-fluoropyrimidin-2-yl)amino)cyclohexyl 4-((4-(3-(2,6-dioxopiperidin-3-yl)-1-methyl-1H-indazol-6-yl)piperidin-1-yl)methyl)-4-hydroxypiperidine-1-carboxylate (Compound 646). To a solution of 3-(6-(1-((4-hydroxypiperidin-4-yl)methyl)piperidin-4-yl)-1-methyl-1H-indazol-3-yl)piperidine-2,6-dione (200 mg, 361 μmol, 1.00 eq, TFA) in DMF (3 mL) was added DIEA (46.6 mg, 361 μmol, 62.9 μL, 1.00 eq) and trans-4-((4-([1,1′-biphenyl]-3-yl)-5-fluoropyrimidin-2-yl)amino)cyclohexyl (4-nitrophenyl) carbonate (95.4 mg, 180 μmol, 0.500 eq) at 25 °C. The mixture was stirred at 25 °C for 1 h. LC-MS showed the desired mass was detected. The reaction mixture was filtered and the filtrate concentrated under high vacuum. The residue was purified by prep-HPLC (Waters Xbridge BEH C18 100*30 mm*10 um; mobile phase: [H₂O (10 mM NH₄HCO₃)-ACN]; gradient: 20%-99% B over 8.0 min) to give the title compound (Compound 646) (23.2 mg, 27.6 μmol, 7.65% yield, 98.7% purity) as a pink solid. LCMS (ES⁺): m/z 829.5 [M+H]⁺.

### Example 650. Synthesis of trans-4-((4-([1,1′-biphenyl]-3-yl)-5-fluoropyrimidin-2-yl)amino)cyclohexyl 4-((4-(3-(2,6-dioxopiperidin-3-yl)-1-methyl-1H-indazol-6-yl)piperazin-1-yl)methyl)piperidine-1-carboxylate (Compound 643)

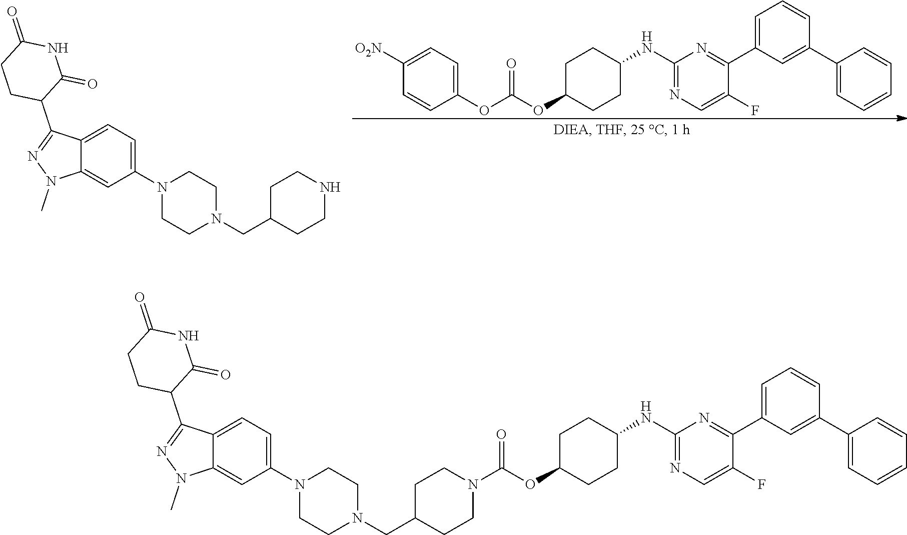 [SMILES: Cn1nc(C2CCC(=O)NC2=O)c2ccc(N3CCN(CC4CCNCC4)CC3)cc21 ; O=C(Oc1ccc([N+](=O)[O-])cc1)O[C@H]1CC[C@H](Nc2ncc(F)c(-c3cccc(-c4ccccc4)c3)n2)CC1 ; Cn1nc(C2CCC(=O)NC2=O)c2ccc(N3CCN(CC4CCN(C(=O)O[C@H]5CC[C@H](Nc6ncc(F)c(-c7cccc(-c8ccccc8)c7)n6)CC5)CC4)CC3)cc21]

Synthesis of trans-4-((4-([1,1′-biphenyl]-3-yl)-5-fluoropyrimidin-2-yl)amino)cyclohexyl 4-((4-(3-(2,6-dioxopiperidin-3-yl)-1-methyl-1H-indazol-6-yl)piperazin-1-yl)methyl)piperidine-1-carboxylate (Compound 643). To a solution of 3-[1-methyl-6-[4-(4-piperidylmethyl)piperazin-1-yl]indazol-3-yl]piperidine-2,6-dione (200 mg, 371 μmol, 1.00 eq, TFA) and trans-[4-[[5-fluoro-4-(3-phenylphenyl)pyrimidin-2-yl]amino]cyclohexyl](4-nitrophenyl) carbonate (98.1 mg, 185 μmol, 0.500 eq) in THF (2 mL) was added DIEA (143 mg, 1.11 mmol, 194 μL, 3.00 eq) at 25 °C. The mixture was stirred at 25 °C for 1 h. LC-MS showed the desired mass was detected. The reaction mixture was concentrated under reduced pressure. The residue was purified by prep-HPLC (column: 3_Phenomenex Luna C18 75*30 mm*3 um; mobile phase: [H₂O (0.2% FA)-ACN]; gradient: 25%-55% B over 8.0 min) to give the title compound (Compound 643) (50.2 mg, 59.8 μmol, 16.1% yield, 98.7% purity, 0.3 FA) as a white solid. LCMS (ES⁺): m/z 814.5 [M+H]⁺.

### Example 651. Synthesis of trans-4-((4-([1,1′-biphenyl]-3-yl)-5-fluoropyrimidin-2-yl)amino)cyclohexyl 4-((4-(3-(2,6-dioxopiperidin-3-yl)-1-methyl-1H-indazol-6-yl)piperazin-1-yl)methyl)-4-hydroxypiperidine-1-carboxylate (Compound 642)

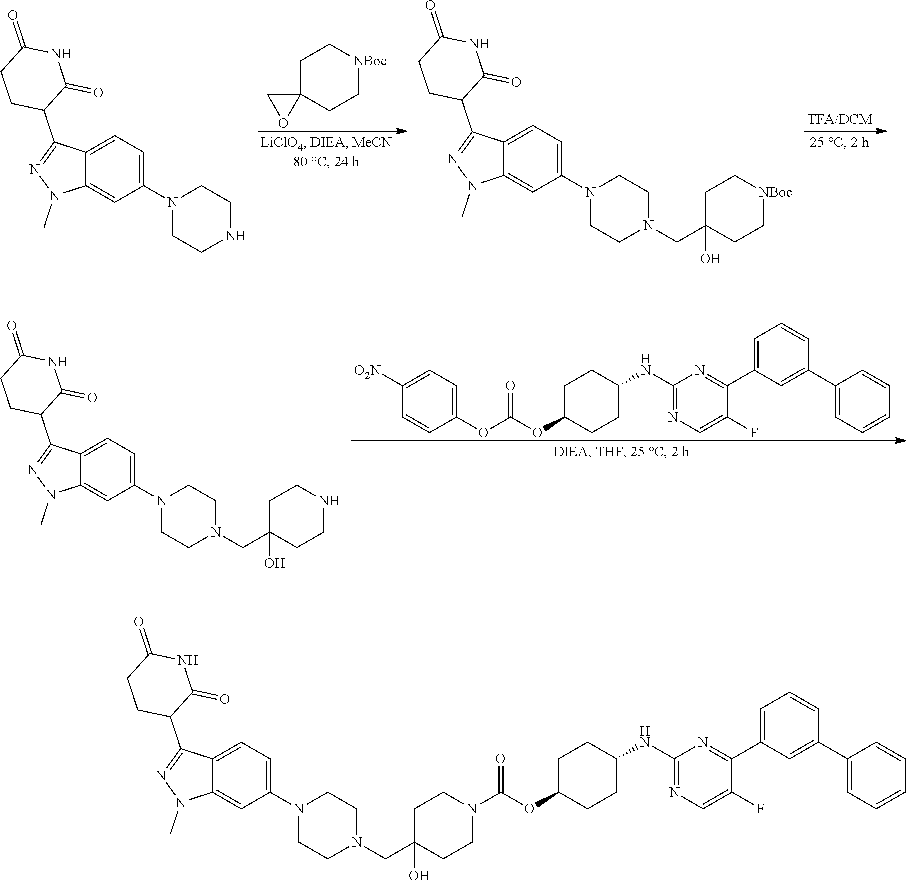 [SMILES: Cn1nc(C2CCC(=O)NC2=O)c2ccc(N3CCNCC3)cc21 ; CC(C)(C)OC(=O)N1CCC2(CC1)CO2 ; Cn1nc(C2CCC(=O)NC2=O)c2ccc(N3CCN(CC4(O)CCN(C(=O)OC(C)(C)C)CC4)CC3)cc21 ; Cn1nc(C2CCC(=O)NC2=O)c2ccc(N3CCN(CC4(O)CCNCC4)CC3)cc21 ; O=C(Oc1ccc([N+](=O)[O-])cc1)O[C@H]1CC[C@H](Nc2ncc(F)c(-c3cccc(-c4ccccc4)c3)n2)CC1 ; Cn1nc(C2CCC(=O)NC2=O)c2ccc(N3CCN(CC4(O)CCN(C(=O)O[C@H]5CC[C@H](Nc6ncc(F)c(-c7cccc(-c8ccccc8)c7)n6)CC5)CC4)CC3)cc21]

Synthesis of 3-(1-methyl-6-(piperazin-1-yl)-1H-indazol-3-yl)piperidine-2,6-dione. To a solution of tert-butyl 4-[3-(2,6-dioxo-3-piperidyl)-1-methyl-indazol-6-yl]piperazine-1-carboxylate (1.00 g, 2.34 mmol, 1.00 eq) in DCM (10 mL) was added TFA (5.12 g, 44.8 mmol, 3.33 mL, 19.1 eq) at 25 °C. The mixture was stirred at 25 °C for 2 h. LC-MS showed desired mass was detected. The reaction mixture was concentrated under reduced pressure to give the title compound (765 mg, crude, TFA) as green oil. LCMS (ES⁺): m/z 328.2 [M+H]⁺.

Synthesis of tert-butyl 4-((4-(3-(2,6-dioxopiperidin-3-yl)-1-methyl-1H-indazol-6-yl)piperazin-1-yl)methyl)-4-hydroxypiperidine-1-carboxylate. To a solution of 3-(1-methyl-6-piperazin-1-yl-indazol-3-yl)piperidine-2,6-dione (700 mg, 1.59 mmol, 1.00 eq, TFA) in ACN (10 mL) was added DIEA (409 mg, 3.17 mmol, 552 μL, 2.00 eq) at 25 °C for 0.5 h, and then added tert-butyl 1-oxa-6-azaspiro[2.5]octane-6-carboxylate (1.01 g, 4.76 mmol, 3.00 eq) and LiClO₄ (253 mg, 2.38 mmol, 104 μL, 1.50 eq) at 25 °C. The mixture was stirred at 80 °C for 23.5 h. LC-MS showed the desired mass was detected. The reaction mixture was quenched by addition saturated Na₂SO₃ solution 10 mL at 0 °C, and then extracted with EtOAc (20 mL*3). The combined organic layers were washed with brine (10 mL), dried over Na₂SO₄, filtered and concentrated under reduced pressure. The residue was purified by column chromatography (SiO₂, Petroleum ether:Tetrahydrofuran=1:1 to 1:2) to give the title compound (1.00 g, 1.48 mmol, 93.3% yield, 80.0% purity) as a brown gum. LCMS (ES⁺): m/z 541.3 [M+H]⁺.

Synthesis of 3-(6-(4-((4-hydroxypiperidin-4-yl)methyl)piperazin-1-yl)-1-methyl-1H-indazol-3-yl)piperidine-2,6-dione. To a solution of tert-butyl 4-[[4-[3-(2,6-dioxo-3-piperidyl)-1-methyl-indazol-6-yl]piperazin-1-yl]methyl]-4-hydroxy-piperidine-1-carboxylate (300 mg, 554 μmol, 1.00 eq) in DCM (1 mL) was added TFA (3.64 g, 31.9 mmol, 2.37 mL, 57.5 eq) at 25 °C. The mixture was stirred at 25 °C for 2 h. LC-MS showed the desired mass was detected. The reaction mixture was concentrated under reduced pressure to give the title compound (240 mg, crude, TFA) as yellow oil. LCMS (ES⁺): m/z 441.2 [M+H]⁺.

Synthesis of trans-4-((4-([1,1′-biphenyl]-3-yl)-5-fluoropyrimidin-2-yl)amino)cyclohexyl 4-((4-(3-(2,6-dioxopiperidin-3-yl)-1-methyl-1H-indazol-6-yl)piperazin-1-yl)methyl)-4-hydroxypiperidine-1-carboxylate (Compound 642). To a solution of 3-[6-[4-[(4-hydroxy-4-piperidyl)methyl]piperazin-1-yl]-1-methyl-indazol-3-yl]piperidine-2,6-dione (200 mg, 360 μmol, 1.00 eq, TFA) and trans-[4-[[5-fluoro-4-(3-phenylphenyl)pyrimidin-2-yl]amino]cyclohexyl](4-nitrophenyl) carbonate (95.3 mg, 180 μmol, 0.500 eq) in THF (2 mL) was added DIEA (139 mg, 1.08 mmol, 188 μL, 3.00 eq) at 25 °C. The mixture was stirred at 25 °C for 2 h. LC-MS showed the desired mass was detected. The reaction mixture was concentrated under reduced pressure. The residue was purified by prep-HPLC (column: 3_Phenomenex Luna C18 75*30 mm*3 um; mobile phase: [H₂O (0.2% FA)-ACN]; gradient: 20%-50% B over 8.0 min) to give the title compound (Compound 642) (57.9 mg, 69.1 μmol, 19.1% yield, 99.1% purity) as a white solid. LCMS (ES⁺): m/z 830.4 [M+H]⁺.

### Example 652. Synthesis of trans-4-((4-([1,1′-biphenyl]-3-yl)-5-fluoropyrimidin-2-yl)amino)cyclohexyl 4-((4-(3-(2,4-dioxotetrahydropyrimidin-1 (2H)-yl)-1-methyl-1H-indazol-6-yl)piperidin-1-yl)methyl)piperidine-1-carboxylate (Compound 636)

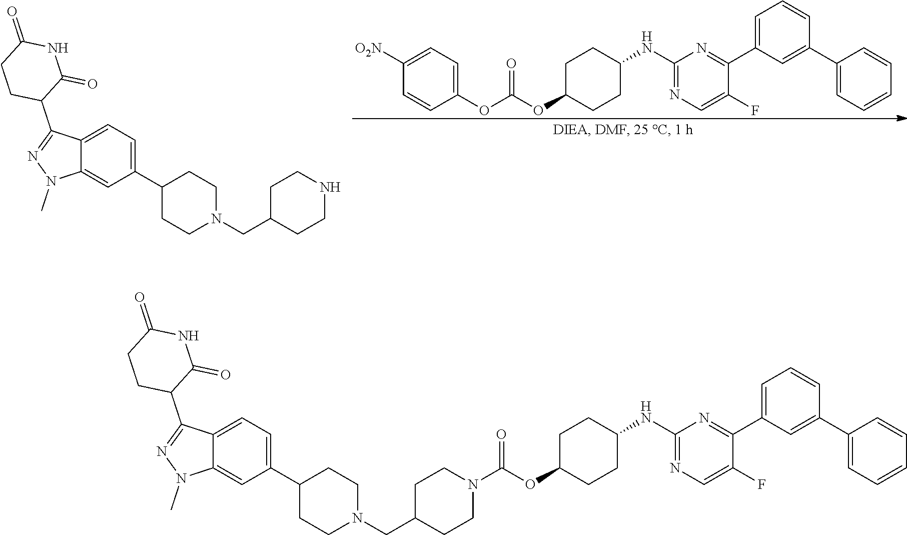 [SMILES: Cn1nc(C2CCC(=O)NC2=O)c2ccc(C3CCN(CC4CCNCC4)CC3)cc21 ; O=C(Oc1ccc([N+](=O)[O-])cc1)O[C@H]1CC[C@H](Nc2ncc(F)c(-c3cccc(-c4ccccc4)c3)n2)CC1 ; Cn1nc(C2CCC(=O)NC2=O)c2ccc(C3CCN(CC4CCN(C(=O)O[C@H]5CC[C@H](Nc6ncc(F)c(-c7cccc(-c8ccccc8)c7)n6)CC5)CC4)CC3)cc21]

Synthesis of trans-4-((4-([1,1′-biphenyl]-3-yl)-5-fluoropyrimidin-2-yl)amino)cyclohexyl 4-((4-(3-(2,4-dioxotetrahydropyrimidin-1 (2H)-yl)-1-methyl-1H-indazol-6-yl)piperidin-1-yl)methyl)piperidine-1-carboxylate (Compound 636). To a solution of 1-(1-methyl-6-(1-(piperidin-4-ylmethyl)piperidin-4-yl)-1H-indazol-3-yl)dihydropyrimidine-2,4 (1H,3H)-dione (180 mg, 334 μmol, 1.00 eq, TFA) in DMF (1 mL) was added DIEA (432 mg, 3.34 mmol, 582 μL, 10.0 eq) and trans-4-((4-([1,1′-biphenyl]-3-yl)-5-fluoropyrimidin-2-yl)amino)cyclohexyl (4-nitrophenyl) carbonate (88.3 mg, 167 μmol, 0.500 eq) at 25 °C. The mixture was stirred at 25 °C for 1 h. LC-MS showed the desired mass was detected. The reaction mixture was filtered and the filtrate concentrated under high vacuum. The residue was purified by prep-HPLC (Waters Xbridge BEH C18 100*30 mm*10 um; mobile phase: [H₂O (10 mM NH₄HCO₃)-ACN]; gradient: 70%-100% B over 8.0 min) to give the title compound (Compound 636) (34.7 mg, 41.4 μmol, 12.4% yield, 97.3% purity) as a white solid. LCMS (ES⁺): m/z 814.5 [M+H]⁺

### Example 653. Synthesis of trans-4-((4-([1,1′-biphenyl]-3-yl)-5-fluoropyrimidin-2-yl)amino)cyclohexyl 4-((4-(3-(2,4-dioxotetrahydropyrimidin-1 (2H)-yl)-1-methyl-1H-indazol-6-yl)piperidin-1-yl)methyl)-4-hydroxypiperidine-1-carboxylate (Compound 635)

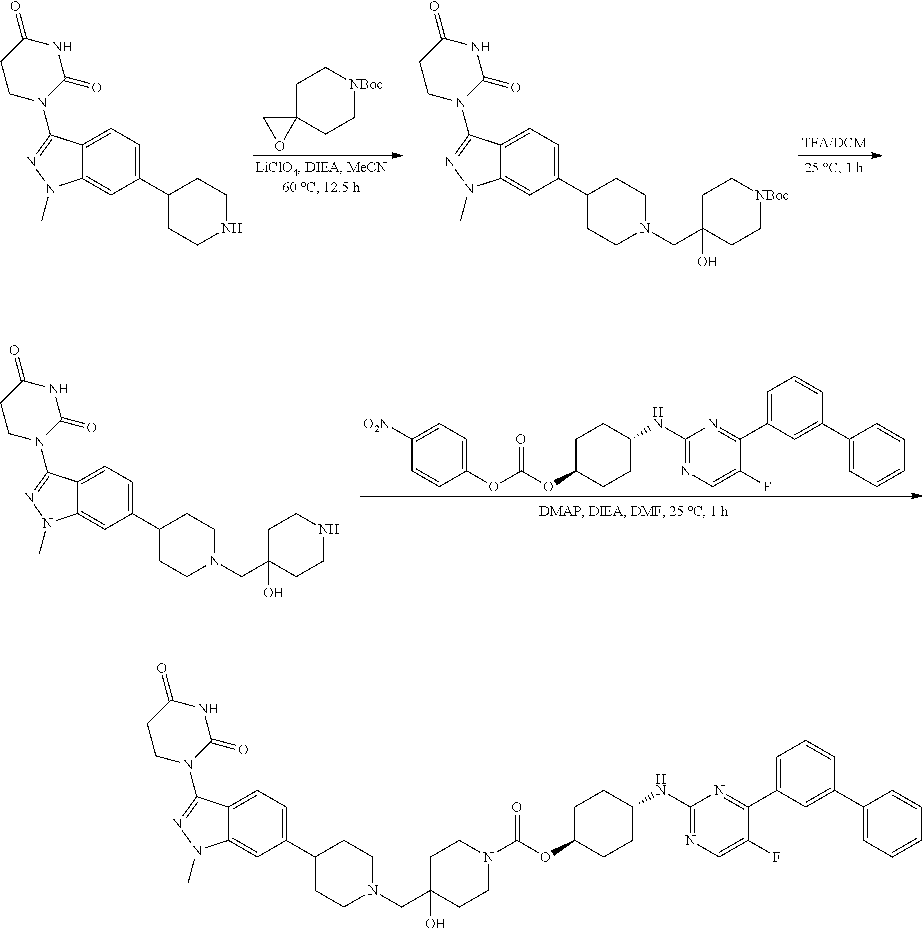 [SMILES: Cn1nc(N2CCC(=O)NC2=O)c2ccc(C3CCNCC3)cc21 ; CC(C)(C)OC(=O)N1CCC2(CC1)CO2 ; Cn1nc(N2CCC(=O)NC2=O)c2ccc(C3CCN(CC4(O)CCN(C(=O)OC(C)(C)C)CC4)CC3)cc21 ; Cn1nc(N2CCC(=O)NC2=O)c2ccc(C3CCN(CC4(O)CCNCC4)CC3)cc21 ; O=C(Oc1ccc([N+](=O)[O-])cc1)O[C@H]1CC[C@H](Nc2ncc(F)c(-c3cccc(-c4ccccc4)c3)n2)CC1 ; Cn1nc(N2CCC(=O)NC2=O)c2ccc(C3CCN(CC4(O)CCN(C(=O)O[C@H]5CC[C@H](Nc6ncc(F)c(-c7cccc(-c8ccccc8)c7)n6)CC5)CC4)CC3)cc21]

Synthesis of tert-butyl 4-((4-(3-(2,4-dioxotetrahydropyrimidin-1 (2H)-yl)-1-methyl-1H-indazol-6-yl)piperidin-1-yl)methyl)-4-hydroxypiperidine-1-carboxylate. To a solution of 1-(1-methyl-6-(piperidin-4-yl)-1H-indazol-3-yl)dihydropyrimidine-2,4 (1H,3H)-dione (450 mg, 1.37 mmol, 1.00 eq) in MeCN (8 mL) was added DIEA (355 mg, 2.75 mmol, 478 μL, 2.00 eq) at 25 °C for 0.5 h. Then tert-butyl 1-oxa-6-azaspiro[2.5]octane-6-carboxylate (879 mg, 4.12 mmol, 3.00 eq) and LiClO₄ (248 mg, 2.34 mmol, 102 μL, 1.70 eq) was added to the mixture at 25 °C. The mixture was stirred at 60 °C for 12 h. LC-MS showed the desired mass was detected. The reaction mixture was quenched by addition saturated Na₂SO₃ solution 10 ml at 0 °C, and extracted with EtOAc (10 mL*3). The combined organic layers dried over Na₂SO₄, filtered and concentrated under reduced pressure. The residue was purified by column chromatography (SiO₂, Petroleum ether:THF=1:0 to 1:1) to give the title compound (750 mg, 1.12 mmol, 81.7% yield, 81.0% purity) as a brown oil. LCMS (ES⁺): m/z 541.3 [M+H]⁺.

Synthesis of 1-(6-(1-((4-hydroxypiperidin-4-yl)methyl)piperidin-4-yl)-1-methyl-1H-indazol-3-yl)dihydropyrimidine-2,4 (1H,3H)-dione. To a solution of tert-butyl 4-((4-(3-(2,4-dioxotetrahydropyrimidin-1 (2H)-yl)-1-methyl-1H-indazol-6-yl)piperidin-1-yl)methyl)-4-hydroxypiperidine-1-carboxylate (250 mg, 462 μmol, 1.00 eq) in DCM (3 mL) was added TFA (1.92 g, 16.8 mmol, 1.25 mL, 36.3 eq) at 25 °C. The mixture was stirred at 25 °C for 1 h. LC-MS showed the desired mass was detected. The reaction mixture was concentrated under reduced pressure to give the title compound (200 mg, crude, TFA) as a yellow oil. LCMS (ES⁺): m/z 441.3 [M+H]⁺.

Synthesis of trans-4-((4-([1,1′-biphenyl]-3-yl)-5-fluoropyrimidin-2-yl)amino)cyclohexyl 4-((4-(3-(2,4-dioxotetrahydropyrimidin-1 (2H)-yl)-1-methyl-1H-indazol-6-yl)piperidin-1-yl)methyl)-4-hydroxypiperidine-1-carboxylate (Compound 635). To a solution of 1-(6-(1-((4-hydroxypiperidin-4-yl)methyl)piperidin-4-yl)-1-methyl-1H-indazol-3-yl)dihydropyrimidine-2,4 (1H,3H)-dione (200 mg, 360 μmol, 1.00 eq, TFA) in DMF (3 mL) was added trans-4-((4-([1,1′-biphenyl]-3-yl)-5-fluoropyrimidin-2-yl)amino)cyclohexyl (4-nitrophenyl) carbonate (190 mg, 360 μmol, 1.00 eq), DMAP (44.0 mg, 360 μmol, 1.00 eq) and DIEA (139 mg, 1.08 mmol, 188 μL, 3.00 eq) at 25 °C. The mixture was stirred at 25 °C for 1 h. LC-MS showed the desired mass was detected. The reaction mixture was filtered to give a residue. The residue was purified by prep-HPLC (column: Phenomenex Luna C18 100*30 mm*3 um; mobile phase: [H₂O (0.2% FA)-ACN]; gradient: 10%-50% B over 8.0 min). The residue was purified by prep-HPLC (column: Waters Xbridge Prep OBD C18 150*40 mm*10 um; mobile phase: [H₂O (10 mM NH₄HCO₃)-ACN]; gradient: 60%-90% B over 8.0 min) to give the title compound (Compound 635) (27.0 mg, 32.5 μmol, 9.02% yield, 100% purity) as a white solid. LCMS (ES⁺): m/z 830.4 [M+H]⁺.

### Example 654. Synthesis of trans-4-((4-([1,1′-biphenyl]-3-yl)-5-fluoropyrimidin-2-yl)amino)cyclohexyl 4-((4-(3-(2,4-dioxotetrahydropyrimidin-1 (2H)-yl)-1-methyl-1H-indazol-6-yl)piperazin-1-yl)methyl)piperidine-1-carboxylate (Compound 634)

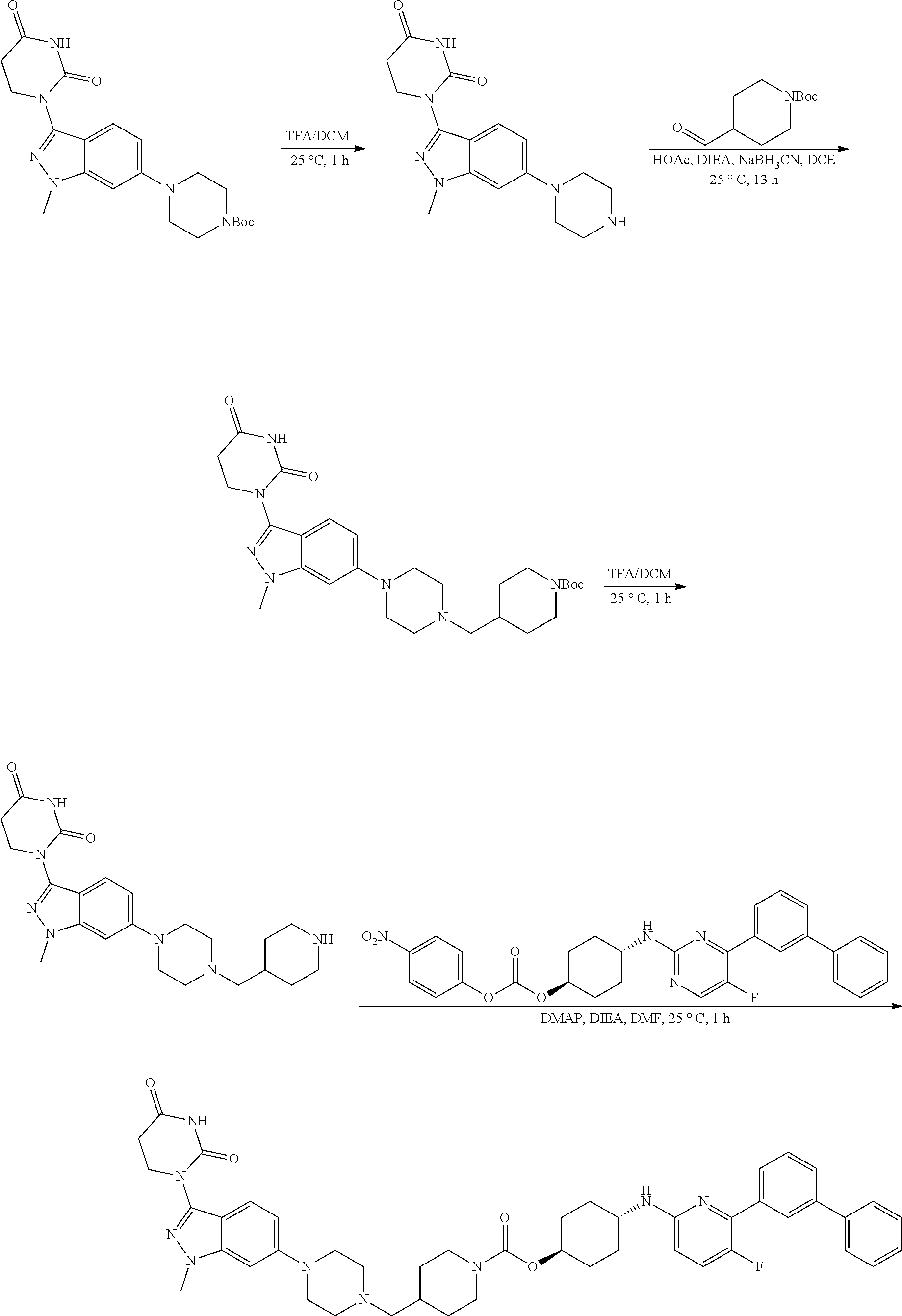

Synthesis of 1-(1-methyl-6-(piperazin-1-yl)-1H-indazol-3-yl)dihydropyrimidine-2,4 (1H,3H)-dione. To a solution of tert-butyl 4-(3-(2,4-dioxotetrahydropyrimidin-1 (2H)-yl)-1-methyl-1H-indazol-6-yl)piperazine-1-carboxylate (200 mg, 466 μmol, 1.00 eq) in DCM (3 mL) was added TFA (1.54 g, 13.4 mmol, 1 mL, 28.8 eq) at 25 °C. The mixture was stirred at 25 °C for 1 h. LC-MS showed the desired mass was detected. The reaction mixture was concentrated under reduced pressure to give the title compound (300 mg, crude, TFA) as a yellow oil. LCMS (ES⁺): m/z 329.2 [M+H]⁺.

Synthesis of tert-butyl 4-((4-(3-(2,4-dioxotetrahydropyrimidin-1 (2H)-yl)-1-methyl-1H-indazol-6-yl)piperazin-1-yl)methyl)piperidine-1-carboxylate. To a solution of 1-(1-methyl-6-(piperazin-1-yl)-1H-indazol-3-yl)dihydropyrimidine-2,4 (1H,3H)-dione (150 mg, 339 μmol, 1.00 eq, TFA) in DCE (5 mL) was added DIEA (131 mg, 1.02 mmol, 177 μL, 3.00 eq) at 25 °C, The mixture was stirred at 25 °C for 0.5 h. Then AcOH (101 mg, 1.70 mmol, 97.0 μL, 5.00 eq) and tert-butyl 4-formylpiperidine-1-carboxylate (216 mg, 1.02 mmol, 3.00 eq) was added to the mixture at 25 °C, The mixture was stirred at 25 °C for 0.5 h. Then NaBH₃CN (106 mg, 1.70 mmol, 5.00 eq) was added to the mixture at 25 °C. The mixture was stirred at 25 °C for 12 h. LC-MS showed the desired mass was detected. The residue was poured into water (5 mL), and extracted with DCM (7 mL*3). The combined organic layers dried over Na₂SO₄, filtered and concentrated under reduced pressure. The residue was purified by column chromatography (SiO₂, Petroleum ether:THF=1:0 to 3:1) to give the title compound (450 mg, crude) as a brown oil. LCMS (ES⁺): m/z 526.4[M+H]⁺.

Synthesis of 1-(1-methyl-6-(4-(piperidin-4-ylmethyl)piperazin-1-yl)-1H-indazol-3-yl)dihydropyrimidine-2,4 (1H,3H)-dione. To a solution of tert-butyl 4-((4-(3-(2,4-dioxotetrahydropyrimidin-1 (2H)-yl)-1-methyl-1H-indazol-6-yl)piperazin-1-yl)methyl)piperidine-1-carboxylate (250 mg, 475 μmol, 1.00 eq) in DCM (3 mL) was added TFA (1.54 g, 13.4 mmol, 1 mL, 28.3 eq) at 25 °C. The mixture was stirred at 25 °C for 1 h. LC-MS showed the desired mass was detected. The reaction mixture was concentrated under reduced pressure to give the title compound (450 mg, crude, TFA) as a yellow oil. LCMS (ES⁺): m/z 426.3[M+H]⁺.

Synthesis of trans-4-((4-([1,1′-biphenyl]-3-yl)-5-fluoropyrimidin-2-yl)amino)cyclohexyl 4-((4-(3-(2,4-dioxotetrahydropyrimidin-1 (2H)-yl)-1-methyl-1H-indazol-6-yl)piperazin-1-yl)methyl)piperidine-1-carboxylate (Compound 634). To a solution of 1-(1-methyl-6-(4-(piperidin-4-ylmethyl)piperazin-1-yl)-1H-indazol-3-yl)dihydropyrimidine-2,4 (1H,3H)-dione (250 mg, 463 μmol, 1.00 eq, TFA) in DMF (3 mL) was added trans-4-((4-([1,1′-biphenyl]-3-yl)-5-fluoropyrimidin-2-yl)amino)cyclohexyl (4-nitrophenyl) carbonate (122 mg, 231 μmol, 0.500 eq), DIEA (179 mg, 1.39 mmol, 242 μL, 3.00 eq) and DMAP (56.6 mg, 463 μmol, 1.00 eq) at 25 °C. The mixture was stirred at 25 °C for 1 h. LC-MS showed the desired mass was detected. The reaction mixture was filtered to give a residue. The residue was purified by prep-HPLC (column: Phenomenex Luna C18 80*30 mm*3 um; mobile phase: [H₂O (0.2% FA)-ACN]; gradient: 30%-60% B over 8.0 min). The residue was purified by prep-HPLC (column: Waters Xbridge BEH C18 100*30 mm*10 um; mobile phase: [H₂O (10 mM NH₄HCO₃)-ACN]; gradient: 35%-75% B over 8.0 min) to give the title compound (Compound 634) (13.1 mg, 16.0 μmol, 3.47% yield, 100% purity) as a white solid. LCMS (ES⁺): m/z 815.4 [M+H]⁺.

### Example 655. Synthesis of trans-(1r,4R)—N-((1r,4R)-4-((4-([1,1′-biphenyl]-3-yl)-5-fluoropyrimidin-2-yl)amino)cyclohexyl)-4-(4-(4-((2,6-dioxopiperidin-3-yl)amino)-2-fluorophenyl)piperazin-1-yl)cyclohexane-1-carboxamide (Compound 604)

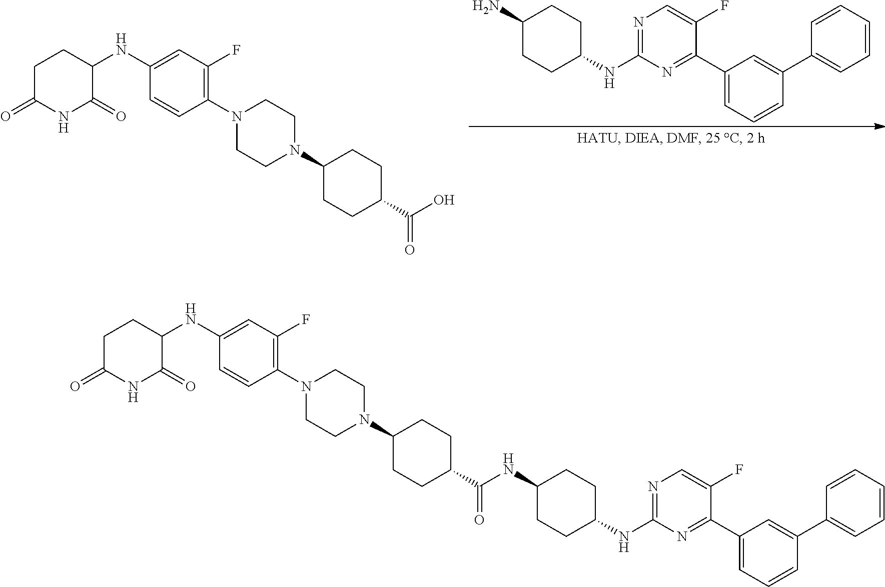 [SMILES: O=C1CCC(Nc2ccc(N3CCN([C@H]4CC[C@H](C(=O)O)CC4)CC3)c(F)c2)C(=O)N1 ; N[C@H]1CC[C@H](Nc2ncc(F)c(-c3cccc(-c4ccccc4)c3)n2)CC1 ; O=C1CCC(Nc2ccc(N3CCN([C@H]4CC[C@H](C(=O)N[C@H]5CC[C@H](Nc6ncc(F)c(-c7cccc(-c8ccccc8)c7)n6)CC5)CC4)CC3)c(F)c2)C(=O)N1]

Synthesis of trans-(1r,4R)—N-((1r,4R)-4-((4-([1,1′-biphenyl]-3-yl)-5-fluoropyrimidin-2-yl)amino)cyclohexyl)-4-(4-(4-((2,6-dioxopiperidin-3-yl)amino)-2-fluorophenyl)piperazin-1-yl)cyclohexane-1-carboxamide (Compound 604). To a solution of trans-4-(4-(4-((2,6-dioxopiperidin-3-yl)amino)-2-fluorophenyl)piperazin-1-yl)cyclohexane-1-carboxylic acid (200 mg, 462 μmol, 1.00 eq) in DMF (4 mL) were added DIEA (179 mg, 1.39 mmol, 242 μL, 3.00 eq), HATU (87.9 mg, 231 μmol, 0.500 eq) and trans-N1-(4-([1,1′-biphenyl]-3-yl)-5-fluoropyrimidin-2-yl)cyclohexane-1,4-diamine (88.1 mg, 185 μmol, 0.400 eq, TFA) at 25 °C. The mixture was stirred at 25 °C for 2 h. LCMS showed trans-4-(4-(4-((2,6-dioxopiperidin-3-yl)amino)-2-fluorophenyl)piperazin-1-yl)cyclohexane-1-carboxylic acid was consumed completely and the desired mass was detected. The reaction mixture was concentrated under reduced pressure to give a residue. The residue were purified by prep-HPLC (column: Phenomenex luna C18 100*40 mm*3 um; mobile phase: [H₂O (0.2% FA)-ACN]; gradient: 15%-55% B over 8.0 min) to give the title compound (Compound 604) (81.5 mg, 95.1 μmol, 20.6% yield, 96.0% purity, FA) as a pale yellow solid. LCMS (ES⁺): m/z 777.4 [M+H]⁺.

### Example 656. Synthesis of trans-(1r,4R)—N-((1r,4R)-4-((4-([1,1′-biphenyl]-3-yl)-5-fluoropyrimidin-2-yl)amino)cyclohexyl)-4-(4-(4-((2,6-dioxopiperidin-3-yl)amino)-2-fluorophenyl)piperidin-1-yl)cyclohexane-1-carboxamide (Compound 660)

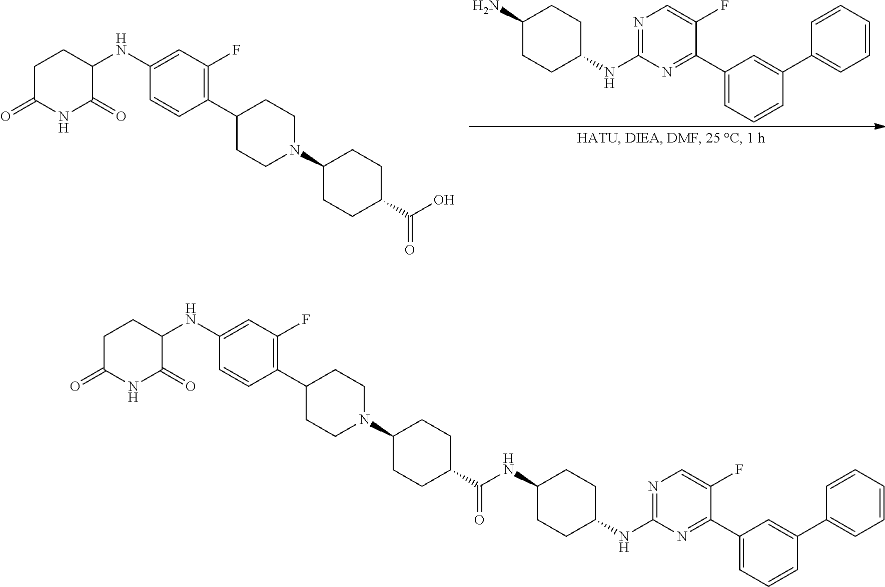 [SMILES: O=C1CCC(Nc2ccc(C3CCN([C@H]4CC[C@H](C(=O)O)CC4)CC3)c(F)c2)C(=O)N1 ; N[C@H]1CC[C@H](Nc2ncc(F)c(-c3cccc(-c4ccccc4)c3)n2)CC1 ; O=C1CCC(Nc2ccc(C3CCN([C@H]4CC[C@H](C(=O)N[C@H]5CC[C@H](Nc6ncc(F)c(-c7cccc(-c8ccccc8)c7)n6)CC5)CC4)CC3)c(F)c2)C(=O)N1]

Synthesis of trans-(1r,4R)—N-((1r,4R)-4-((4-([1,1′-biphenyl]-3-yl)-5-fluoropyrimidin-2-yl)amino)cyclohexyl)-4-(4-(4-((2,6-dioxopiperidin-3-yl)amino)-2-fluorophenyl)piperidin-1-yl)cyclohexane-1-carboxamide (Compound 660). To a solution of trans-4-(4-(4-((2,6-dioxopiperidin-3-yl)amino)-2-fluorophenyl)piperidin-1-yl)cyclohexane-1-carboxylic acid (170 mg, 394 μmol, 1.00 eq) in DMF (5 mL) were added HATU (74.9 mg, 197 μmol, 0.500 eq), DIEA (255 mg, 1.97 mmol, 343 μL, 5.00 eq) and trans-N4-[5-fluoro-4-(3-phenylphenyl)pyrimidin-2-yl]cyclohexane-1,4-diamine (93.9 mg, 197 μmol, 0.500 eq, TFA) at 25 °C. The mixture was stirred at 25 °C for 1 h. LCMS showed trans-4-(4-(4-((2,6-dioxopiperidin-3-yl)amino)-2-fluorophenyl)piperidin-1-yl)cyclohexane-1-carboxylic acid was consumed completely and the desired mass was detected. The reaction mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 20%-60% B over 8.0 min) to give the title compound (Compound 660) (87.4 mg, 105 μmol, 26.6% yield, 98.5% purity, FA) as a pale yellow solid. LCMS (ES⁺): m/z 776.4 [M+H]⁺.

### Example 657. Synthesis of trans-N-(4-((4-([1,1′-biphenyl]-3-yl)-5-fluoropyrimidin-2-yl)amino)cyclohexyl)-1′-(4-((2,6-dioxopiperidin-3-yl)amino)-2-fluorophenyl)-[1,4′-bipiperidine]-4-carboxamide (Compound 649)

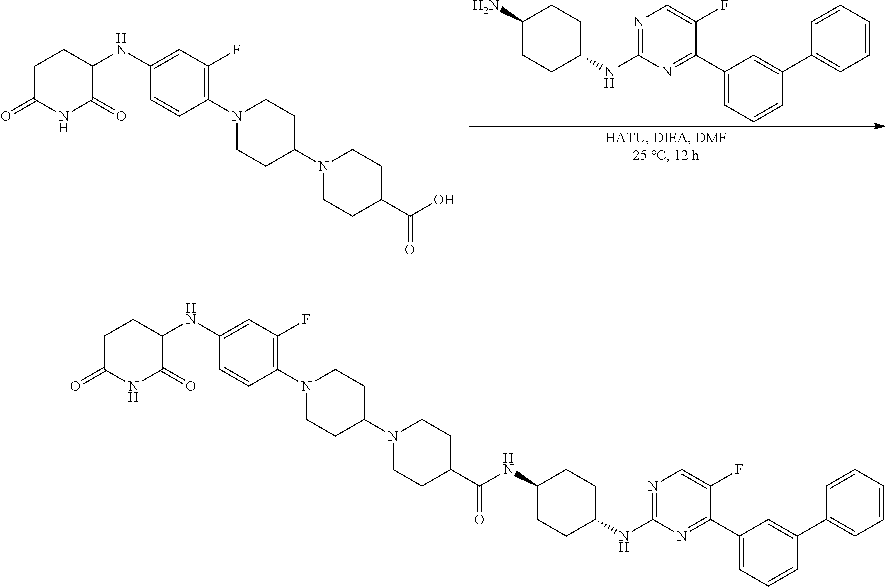 [SMILES: O=C1CCC(Nc2ccc(N3CCC(N4CCC(C(=O)O)CC4)CC3)c(F)c2)C(=O)N1 ; N[C@H]1CC[C@H](Nc2ncc(F)c(-c3cccc(-c4ccccc4)c3)n2)CC1 ; O=C1CCC(Nc2ccc(N3CCC(N4CCC(C(=O)N[C@H]5CC[C@H](Nc6ncc(F)c(-c7cccc(-c8ccccc8)c7)n6)CC5)CC4)CC3)c(F)c2)C(=O)N1]

Synthesis of trans-N-(4-((4-([1,1′-biphenyl]-3-yl)-5-fluoropyrimidin-2-yl)amino)cyclohexyl)-1′-(4-((2,6-dioxopiperidin-3-yl)amino)-2-fluorophenyl)-[1,4′-bipiperidine]-4-carboxamide (Compound 649). To a solution of 1′-(4-((2,6-dioxopiperidin-3-yl)amino)-2-fluorophenyl)-[1,4′-bipiperidine]-4-carboxylic acid (130 mg, 301 μmol, 1.00 eq) in DMF (3 mL) were added HATU (79.8 mg, 210 μmol, 0.700 eq) and DIEA (194 mg, 1.50 mmol, 260 μL, 5.00 eq) at 25 °C, and then trans-N1-(4-([1,1′-biphenyl]-3-yl)-5-fluoropyrimidin-2-yl)cyclohexane-1,4-diamine (100 mg, 210 μmol, 0.700 eq, TFA) was added at 25 °C. The resulting mixture was stirred at 25 °C for 12 h. LCMS showed 1′-(4-((2,6-dioxopiperidin-3-yl) amino)-2-fluorophenyl)-[1,4′-bipiperidine]-4-carboxylic acid was consumed and desired mass was detected. The mixture was concentrated under reduced pressure to give a residue. The residue was purified by prep-HPLC (column: 2_Phenomenex Gemini C18 75*40 mm*3 um; mobile phase: [H₂O (10 mM NH₄HCO₃)-ACN]; gradient: 40%-70% B over 8.0 min) to give the title compound (Compound 649) (7.90 mg, 9.80 μmol, 3.27% yield, 96.4% purity) as a white solid. LCMS (ESI+): m/s 777.4 [M+H]⁺

### Example 658. Synthesis of N-((1r,4r)-4-((4-([1,1′-biphenyl]-3-yl)-5-fluoropyrimidin-2-yl)amino)cyclohexyl)-1′-(4-((2,6-dioxopiperidin-3-yl)amino)-2-fluorophenyl)-3′-fluoro-[1,4′-bipiperidine]-4-carboxamide (Compound 656)

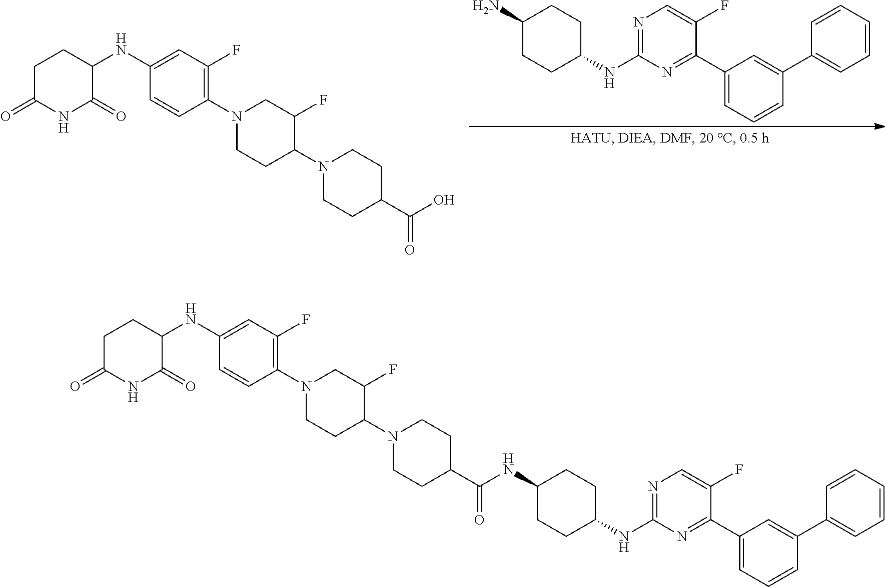 [SMILES: O=C1CCC(Nc2ccc(N3CCC(N4CCC(C(=O)O)CC4)C(F)C3)c(F)c2)C(=O)N1 ; N[C@H]1CC[C@H](Nc2ncc(F)c(-c3cccc(-c4ccccc4)c3)n2)CC1 ; O=C1CCC(Nc2ccc(N3CCC(N4CCC(C(=O)N[C@H]5CC[C@H](Nc6ncc(F)c(-c7cccc(-c8ccccc8)c7)n6)CC5)CC4)C(F)C3)c(F)c2)C(=O)N1]

Synthesis of N-((1r,4r)-4-((4-([1,1′-biphenyl]-3-yl)-5-fluoropyrimidin-2-yl)amino)cyclohexyl)-1′-(4-((2,6-dioxopiperidin-3-yl)amino)-2-fluorophenyl)-3′-fluoro-[1,4′-bipiperidine]-4-carboxamide (Compound 656). To a solution of 1′-(4-((2,6-dioxopiperidin-3-yl)amino)-2-fluorophenyl)-3′-fluoro-[1,4′-bipiperidine]-4-carboxylic acid (177 mg, 393 μmol, 1.00 eq) in DMF (4 mL) were added HATU (89.6 mg, 236 μmol, 0.600 eq) and DIEA (255 mg, 1.97 mmol, 343 μL, 5.00 eq) at 20 °C, then trans-N1-(4-([1,1′-biphenyl]-3-yl)-5-fluoropyrimidin-2-yl)cyclohexane-1,4-diamine (112 mg, 236 μmol, 0.600 eq, TFA) was added to the mixture at 20 °C, the mixture was stirred at 20 °C for 0.5 h. LCMS showed 1′-(4-((2,6-dioxopiperidin-3-yl)amino)-2-fluorophenyl)-3′-fluoro-[1,4′-bipiperidine]-4-carboxylic acid was consumed completely and the desired mass was detected. The mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 25%-65% B over 8.0 min) to give the title compound (Compound 656) (61.2 mg, 70.7 μmol, 18.0% yield, 97.1% purity, FA) as a yellow solid. LCMS (ES⁺): m/z 795.4 [M+H]⁺

### Example 659. Synthesis of trans-(1r,4R)-4-((4-([1,1′-biphenyl]-3-yl)-5-fluoropyrimidin-2-yl)amino)-N-((1r,4R)-4-(4-(4-((2,6-dioxopiperidin-3-yl)amino)-2-fluorophenyl)piperazin-1-yl)cyclohexyl)cyclohexane-1-carboxamide (Compound 662)

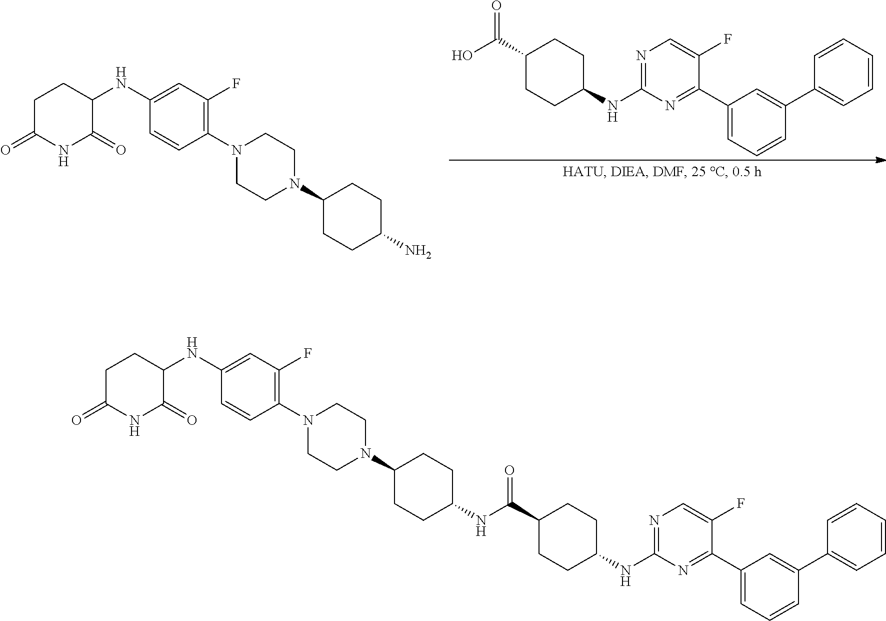 [SMILES: N[C@H]1CC[C@H](N2CCN(c3ccc(NC4CCC(=O)NC4=O)cc3F)CC2)CC1 ; O=C(O)[C@H]1CC[C@H](Nc2ncc(F)c(-c3cccc(-c4ccccc4)c3)n2)CC1 ; O=C1CCC(Nc2ccc(N3CCN([C@H]4CC[C@H](NC(=O)[C@H]5CC[C@H](Nc6ncc(F)c(-c7cccc(-c8ccccc8)c7)n6)CC5)CC4)CC3)c(F)c2)C(=O)N1]

Synthesis of trans-(1r,4R)-4-((4-([1,1′-biphenyl]-3-yl)-5-fluoropyrimidin-2-yl)amino)-N-((1r,4R)-4-(4-(4-((2,6-dioxopiperidin-3-yl)amino)-2-fluorophenyl)piperazin-1-yl)cyclohexyl)cyclohexane-1-carboxamide (Compound 662). To a solution of trans-4-((4-([1,1′-biphenyl]-3-yl)-5-fluoropyrimidin-2-yl)amino)cyclohexane-1-carboxylic acid (93.3 mg, 238 μmol, 0.400 eq) in DMF (5 mL) were added HATU (113 mg, 298 μmol, 0.500 eq), DIEA (385 mg, 2.98 mmol, 1.04 mL, 5.00 eq) and trans-3-((4-(4-(4-aminocyclohexyl)piperazin-1-yl)-3-fluorophenyl)amino)piperidine-2,6-dione (308 mg, 596 μmol, 1.00 eq, TFA) at 25 °C. The mixture was stirred at 25 °C for 0.5 h. LCMS showed trans-3-((4-(4-(4-aminocyclohexyl)piperazin-1-yl)-3-fluorophenyl)amino)piperidine-2,6-dione was consumed completely and the desired mass was detected. The reaction mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 25%-60% B over 8.0 min) to give the title compound (Compound 662) (85.8 mg, 101 μmol, 17.0% yield, 91.5% purity) as a yellow solid. LCMS (ES⁺): m/z 777.4 [M+H]⁺

### Example 660. Synthesis of trans-(1r,4R)-4-((4-([1,1′-biphenyl]-3-yl)-5-fluoropyrimidin-2-yl)amino)-N-((1r,4R)-4-(4-(4-((2,6-dioxopiperidin-3-yl)amino)-2-fluorophenyl)piperidin-1-yl)cyclohexyl)cyclohexane-1-carboxamide (Compound 658)

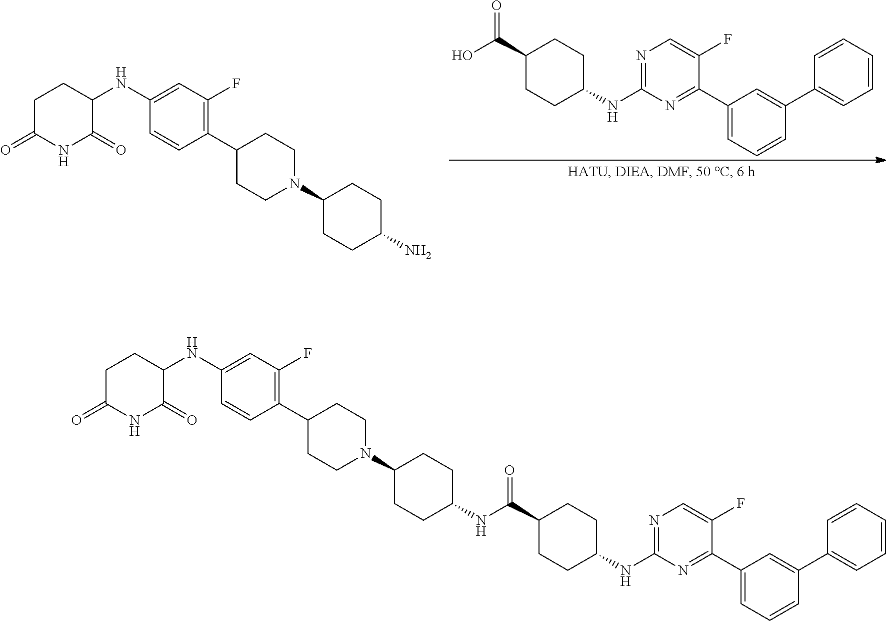 [SMILES: N[C@H]1CC[C@H](N2CCC(c3ccc(NC4CCC(=O)NC4=O)cc3F)CC2)CC1 ; O=C(O)[C@H]1CC[C@H](Nc2ncc(F)c(-c3cccc(-c4ccccc4)c3)n2)CC1 ; O=C1CCC(Nc2ccc(C3CCN([C@H]4CC[C@H](NC(=O)[C@H]5CC[C@H](Nc6ncc(F)c(-c7cccc(-c8ccccc8)c7)n6)CC5)CC4)CC3)c(F)c2)C(=O)N1]

Synthesis of trans-(1r,4R)-4-((4-([1,1′-biphenyl]-3-yl)-5-fluoropyrimidin-2-yl)amino)-N-((1r,4R)-4-(4-(4-((2,6-dioxopiperidin-3-yl)amino)-2-fluorophenyl)piperidin-1-yl)cyclohexyl)cyclohexane-1-carboxamide (Compound 658). To a solution of trans-4-((4-([1,1′-biphenyl]-3-yl)-5-fluoropyrimidin-2-yl)amino)cyclohexane-1-carboxylic acid (45.5 mg, 116 μmol, 0.500 eq) in DMF (3 mL) were added HATU (177 mg, 464 μmol, 2.00 eq) and DIEA (60.1 mg, 465 μmol, 80.9 μL, 2.00 eq) at 25 °C. After addition, trans-3-((4-(1-(4-aminocyclohexyl)piperidin-4-yl)-3-fluorophenyl)amino)piperidine-2,6-dione (120 mg, 232 μmol, 1.00 eq, TFA) was added at 25 °C. The resulting mixture was stirred at 50 °C for 6 h. LCMS showed trans-4-((4-([1,1′-biphenyl]-3-yl)-5-fluoropyrimidin-2-yl)amino)cyclohexane-1-carboxylic acid was consumed and the desired mass was detected. The mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 20%-60% B over 8.0 min) to give the title compound (Compound 658) (23.9 mg, 29.2 μmol, 12.6% yield, 94.9% purity) as a yellow solid. LCMS (ESI+): m/s 776.4 [M+H]⁺.

### Example 661. Synthesis of trans-3-((3-fluoro-4-(1-((4-(4-((5-fluoro-4-(3-morpholinophenyl)pyrimidin-2-yl)amino)piperidine-1-carbonyl)cyclohexyl)methyl)piperidin-4-yl)phenyl)amino)piperidine-2,6-dione (Compound 671)

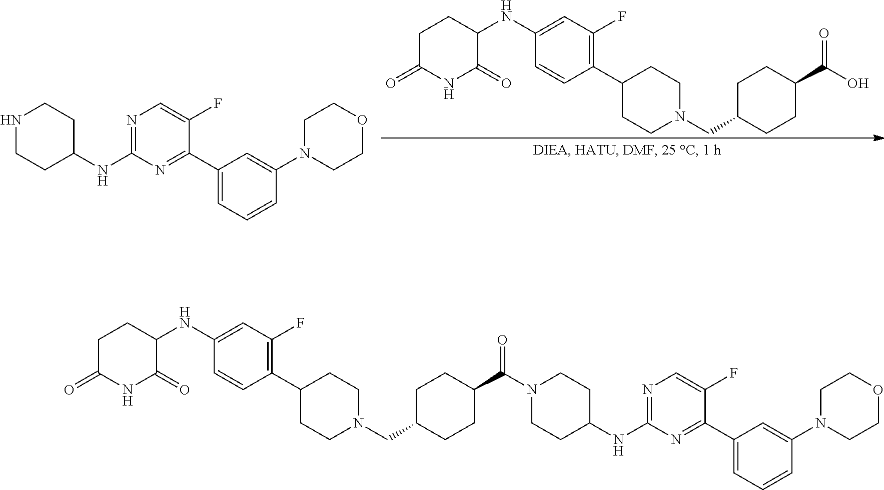

Synthesis of trans-3-((3-fluoro-4-(1-((4-(4-((5-fluoro-4-(3-morpholinophenyl)pyrimidin-2-yl)amino)piperidine-1-carbonyl)cyclohexyl)methyl)piperidin-4-yl)phenyl)amino)piperidine-2,6-dione (Compound 671). To a solution of trans-4-((4-(4-((2,6-dioxopiperidin-3-yl)amino)-2-fluorophenyl)piperidin-1-yl)methyl)cyclohexane-1-carboxylic acid (178 mg, 399 μmol, 1.00 eq) in DMF (2 mL) were added DIEA (258 mg, 1.99 mmol, 348 μL, 5.00 eq) and HATU (152 mg, 399 μmol, 1.00 eq) at 25 °C. Then to the mixture was added 5-fluoro-4-(3-morpholinophenyl)-N-(piperidin-4-yl)pyrimidin-2-amine (113 mg, 239 μmol, 0.600 eq, TFA) at 25 °C. The mixture was stirred at 25 °C for 1 h. LCMS showed trans-4-((4-(4-((2,6-dioxopiperidin-3-yl)amino)-2-fluorophenyl)piperidin-1-yl)methyl)cyclohexane-1-carboxylic acid was consumed and the desired mass was detected. The mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 15%-50% B over 8.0 min) to give the title compound (Compound 671) (71.5 mg, 89.7 μmol, 22.5% yield, 98.5% purity, FA) as a pale yellow solid. LCMS (ES⁺): m/z 785.5 [M+H]⁺.

### Example 662. Synthesis of 3-((3-fluoro-4-(4-((trans-4-(4-((5-fluoro-4-(3-morpholinophenyl) pyrimidin-2-yl) amino) piperidine-1-carbonyl) cyclohexyl) methyl) piperazin-1-yl) phenyl) amino) piperidine-2,6-dione (Compound 670)

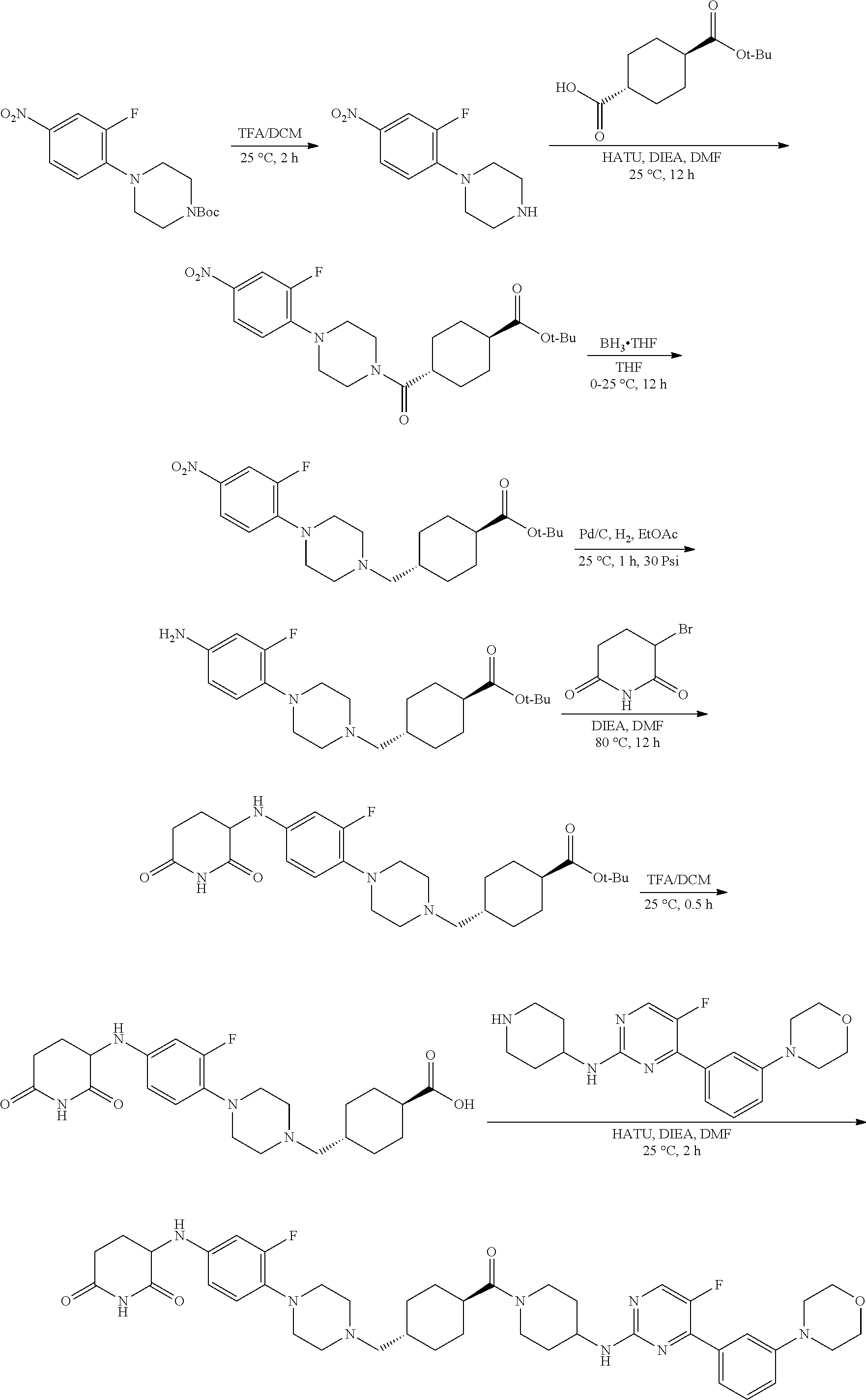

Synthesis of 1-(2-fluoro-4-nitrophenyl) piperazine. To a solution of tert-butyl 4-(2-fluoro-4-nitrophenyl) piperazine-1-carboxylate (5.00 g, 15.4 mmol, 1.00 eq) in DCM (50 mL) was added TFA (10 mL) at 25 °C. The resulting mixture was stirred at 25 °C for 2 h. LCMS showed that tert-butyl 4-(2-fluoro-4-nitrophenyl) piperazine-1-carboxylate was consumed completely and the desired mass was detected. The mixture was concentrated under reduced pressure to give the title compound (3.46 g, crude, TFA) as a yellow oil. LCMS (ES⁺): m/z 226.1 [M+H]⁺.

Synthesis of trans-tert-butyl-4-(4-(2-fluoro-4-nitrophenyl) piperazine-1-carbonyl) cyclohexane-1-carboxylate. To a solution of trans-4-(tert-butoxycarbonyl) cyclohexane-1-carboxylic acid (2.33 g, 10.2 mmol, 1.00 eq) in DMF (40 mL) were added HATU (3.88 g, 10.2 mmol, 1.00 eq) and DIEA (2.64 g, 20.4 mmol, 3.55 mL, 2.00 eq) at 25 °C, and then 1-(2-fluoro-4-nitrophenyl) piperazine (3.46 g, 10.2 mmol, 1.00 eq, TFA) was added at 25 °C. The resulting mixture was stirred at 25 °C for 12 h. LCMS showed that 1-(2-fluoro-4-nitrophenyl) piperazine was consumed completely and the desired mass was detected. H₂O (50 mL) was added to the solution, then the mixture was extracted with EtOAc (50 mL*3), the combined organic layers were dried over Na₂SO₄, filtered and the filtrate was concentrated under reduced pressure to give a residue. The residue was purified by MPLC (SiO₂, Petroleum ether/Ethyl acetate=1/0 to 1/1) to give the title compound (4.40 g, 10.1 mmol, 99.1% yield) as a yellow solid. LCMS (ES⁺): m/z 436.3 [M+H]⁺.

Synthesis of trans-tert-butyl-4-((4-(2-fluoro-4-nitrophenyl) piperazin-1-yl) methyl) cyclohexane-1-carboxylate. To a solution of trans-tert-butyl-4-(4-(2-fluoro-4-nitrophenyl) piperazine-1-carbonyl) cyclohexane-1-carboxylate (1.13 g, 2.59 mmol, 1.00 eq) in THF (20 mL) was added BH₃·THF (1 M in THF, 7.78 mL, 3.00 eq) at 0 °C under N₂, the mixture was stirred at 25 °C for 12 h under N₂ atmosphere. LCMS showed that trans-tert-butyl-4-(4-(2-fluoro-4-nitrophenyl) piperazine-1-carbonyl) cyclohexane-1-carboxylate was consumed completely and the desired mass was detected. The borane was quenched by slowly adding methanol (10 mL) to the reaction system at 0 °C. And H₂O (20 mL) was added to the solution, then the mixture was extracted with EtOAc (40 mL*3), the combined organic layers were dried over Na₂SO₄, filtered and the filtrate was concentrated under reduced pressure to give a residue. The residue was purified by MPLC (SiO₂, Petroleum ether/Ethyl acetate=1/0 to 1/1) to give the title compound (1.00 g, 2.37 mmol, 91.4% yield) as a yellow solid. LCMS (ES⁺): m/z 422.3 [M+H]⁺.

Synthesis of trans-tert-butyl-4-((4-(4-amino-2-fluorophenyl)piperazin-1-yl)methyl)cyclohexane-1-carboxylate. To 10% Pd/C (1.00 g, 50% purity) was added EtOAc (30 mL) at 25 °C under N₂. Then trans-tert-butyl-4-((4-(2-fluoro-4-nitrophenyl) piperazin-1-yl) methyl) cyclohexane-1-carboxylate (1.00 g, 2.37 mmol, 1.00 eq) was added to the mixture at 25 °C under N₂. The mixture was degassed and purged with H₂ for 3 times and the resulting mixture was stirred at 25 °C for 1 h under H₂ (30 psi). LCMS showed that trans-tert-butyl-4-((4-(2-fluoro-4-nitrophenyl) piperazin-1-yl) methyl) cyclohexane-1-carboxylate was consumed completely and the desired mass was detected. The mixture was filtered, the filtrate was concentrated under reduced pressure to give the title compound (1.05 g, crude) as a brown solid. LCMS (ES⁺): m/z 392.3 [M+H]⁺.

Synthesis of trans-tert-butyl-4-((4-(4-((2,6-dioxopiperidin-3-yl) amino)-2-fluorophenyl) piperazin-1-yl) methyl) cyclohexane-1-carboxylate. To a solution of trans-tert-butyl-4-((4-(4-amino-2-fluorophenyl) piperazin-1-yl) methyl) cyclohexane-1-carboxylate (1.05 g, 2.68 mmol, 1.00 eq) and 3-bromopiperidine-2,6-dione (1.54 g, 8.05 mmol, 3.00 eq) in DMF (30 mL) was added DIEA (1.04 g, 8.05 mmol, 1.40 mL, 3.00 eq) at 25 °C. The resulting mixture was stirred at 80 °C for 12 h. LCMS showed that trans-tert-butyl-4-((4-(4-amino-2-fluorophenyl) piperazin-1-yl) methyl) cyclohexane-1-carboxylate was consumed completely and the desired mass was detected. H₂O (30 mL) was added to the solution, then the mixture was extracted with EtOAc (30 mL*3), the combined organic layers were washed with brine (10 mL*3), the combined organic layers were dried over Na₂SO₄, filtered and the filtrate was concentrated under reduced pressure to give a residue. The residue was purified by MPLC (SiO₂, Petroleum ether/Ethyl acetate=1/0 to 1/1) to give the title compound (350 mg, 696 μmol, 25.8% yield) as a brown solid. LCMS (ES⁺): m/z 503.4 [M+H]⁺.

Synthesis of trans-4-((4-(4-((2,6-dioxopiperidin-3-yl) amino)-2-fluorophenyl) piperazin-1-yl) methyl) cyclohexane-1-carboxylic acid. To a solution of trans-tert-butyl-4-((4-(4-((2,6-dioxopiperidin-3-yl) amino)-2-fluorophenyl) piperazin-1-yl) methyl) cyclohexane-1-carboxylate (100 mg, 199 μmol, 1.00 eq) in DCM (3 mL) was added TFA (1 mL) at 25 °C. The resulting mixture was stirred at 25 °C for 0.5 h. LCMS showed that trans-tert-butyl-4-((4-(4-((2,6-dioxopiperidin-3-yl) amino)-2-fluorophenyl) piperazin-1-yl) methyl) cyclohexane-1-carboxylate was consumed completely and the desired mass was detected. The mixture was concentrated under reduced pressure to give the title compound (100 mg, crude) as a yellow oil. LCMS (ES⁺): m/z 447.3 [M+H]⁺.

Synthesis of trans-3-((3-fluoro-4-(4-((4-(4-((5-fluoro-4-(3-morpholinophenyl) pyrimidin-2-yl) amino) piperidine-1-carbonyl) cyclohexyl) methyl) piperazin-1-yl) phenyl) amino) piperidine-2,6-dione (Compound 670). To a solution of trans-4-((4-(4-((2,6-dioxopiperidin-3-yl) amino)-2-fluorophenyl) piperazin-1-yl) methyl) cyclohexane-1-carboxylic acid (90.0 mg, 208 μmol, 1.00 eq) in DMF (4 mL) were added HATU (79.1 mg, 208 μmol, 1.00 eq) and DIEA (108 mg, 832 μmol, 145 μL, 4.00 eq) at 25 °C. Then 5-fluoro-4-(3-morpholinophenyl)-N-(piperidin-4-yl) pyrimidin-2-amine (74.4 mg, 158 μmol, 0.800 eq, TFA) was added at 25 °C. The resulting mixture was stirred at 25 °C for 2 h. LCMS showed that trans-4-((4-(4-((2,6-dioxopiperidin-3-yl) amino)-2-fluorophenyl) piperazin-1-yl) methyl) cyclohexane-1-carboxylic acid was consumed completely and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 35%-65% B over 8.0 min) to give the title compound (Compound 670) (10.5 mg, 12.7 μmol, 6.08% yield, 94.7% purity) as a yellow solid. LCMS (ESI+): m/z 786.4 [M+H]⁺

### Example 663. Synthesis of trans-3-((3-fluoro-4-(1-((4-(4-((5-fluoro-4-(3-(2-oxo-1,3-oxazinan-3-yl)phenyl)pyrimidin-2-yl)amino)piperidine-1-carbonyl)cyclohexyl)methyl)piperidin-4-yl)phenyl)amino)piperidine-2,6-dione (Compound 684)

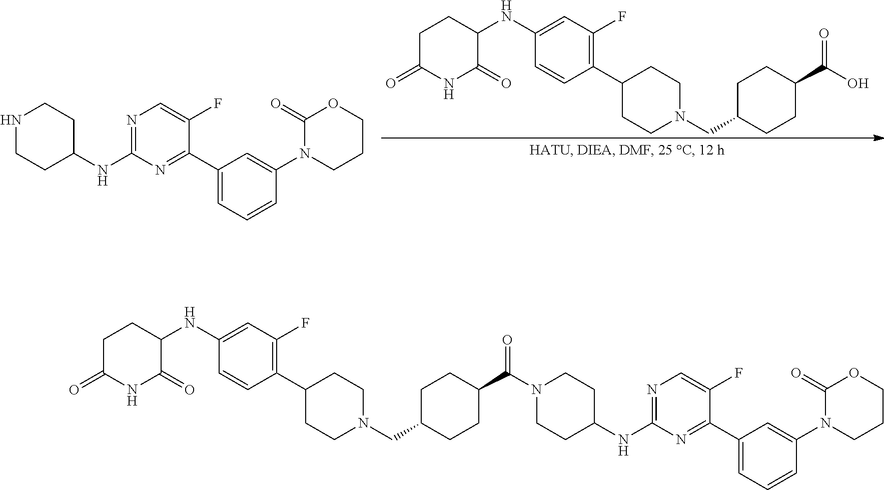

Synthesis of trans-3-((3-fluoro-4-(1-((4-(4-((5-fluoro-4-(3-(2-oxo-1,3-oxazinan-3-yl)phenyl)pyrimidin-2-yl)amino)piperidine-1-carbonyl)cyclohexyl)methyl)piperidin-4-yl)phenyl)amino)piperidine-2,6-dione (Compound 684). To a solution of 3-[3-[5-fluoro-2-(4-piperidylamino)pyrimidin-4-yl]phenyl]-1,3-oxazinan-2-one (59.7 mg, 146 μmol, 5.46e-1 eq, HCl) and trans-4-[[4-[4-[(2,6-dioxo-3-piperidyl)amino]-2-fluoro-phenyl]-1-piperidyl]methyl]cyclohexanecarboxylic acid (150 mg, 268 μmol, 1.00 eq, TFA) in DMF (2 mL) was added DIEA (103 mg, 804 μmol, 140 μL, 3.00 eq) at 25 °C. The mixture was stirred at 25 °C for 1 h, then added HATU (101 mg, 268 μmol, 1.00 eq) to the reaction solution at 25 °C, the mixture was stirred at 25 °C for 11 h. LC-MS showed the desired mass was detected. The reaction mixture was concentrated under reduced pressure. The residue was purified by prep-HPLC (column: Phenomenex luna C18 100*40 mm*5 um; mobile phase: [H₂O (0.04% HCl)-ACN]; gradient: 10%-35% B over 8.0 min) to give desired compound as a pale yellow solid. The residue was purified by prep-HPLC (column: Waters Xbridge BEH C18 100*30 mm*10 um; mobile phase: [H₂O (10 mM NH₄HCO₃)-ACN]; gradient: 20%-80% B over 8.0 min) to give the title compound (Compound 684) (10.0 mg, 12.3 μmol, 4.61% yield, 98.7% purity) as a pale yellow solid. LCMS (ES+): m/z 799.4 [M+H]+.

### Example 664. Synthesis of 3-((3-fluoro-4-(4-((trans-4-(4-((5-fluoro-4-(3-(2-oxo-1,3-oxazinan-3-yl)phenyl)pyrimidin-2-yl)amino)piperidine-1-carbonyl)cyclohexyl)methyl)piperazin-1-yl)phenyl)amino)piperidine-2,6-dione (Compound 667)

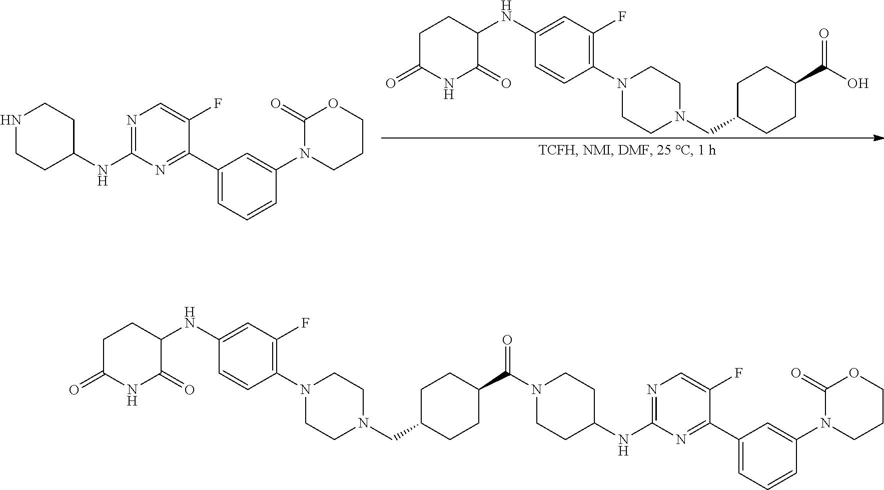

Synthesis of trans-3-((3-fluoro-4-(4-((4-(4-((5-fluoro-4-(3-(2-oxo-1,3-oxazinan-3-yl)phenyl)pyrimidin-2-yl)amino)piperidine-1-carbonyl)cyclohexyl)methyl)piperazin-1-yl)phenyl)amino)piperidine-2,6-dione (Compound 667). To a solution of 3-[3-[5-fluoro-2-(4-piperidylamino)pyrimidin-4-yl]phenyl]-1,3-oxazinan-2-one (250 mg, 515 μmol, 1.00 eq, TFA) in DMF (1 mL) was added NMI (126 mg, 1.55 mmol, 123 μL, 3.00 eq), trans-4-((4-(4-((2,6-dioxopiperidin-3-yl)amino)-2-fluorophenyl)piperazin-1-yl)methyl)cyclohexane-1-carboxylic acid (229 mg, 410 μmol, 0.797 eq, TFA) and TCFH (216 mg, 772 μmol, 1.50 eq) at 25 °C. The mixture was stirred at 25 °C for 1 h. LC-MS showed the desired mass was detected. The reaction mixture was concentrated under reduced pressure. The residue was purified by prep-HPLC (column: Phenomenex luna C18 100*40 mm*5 um; mobile phase: [H₂O (0.2% FA)-ACN]; gradient: 5%-45% B over 8.0 min). The residue was again purified by prep-HPLC (column: Waters Xbridge BEH C18 100*30 mm*10 um; mobile phase: [H₂O (10 mM NH4HCO3)-ACN]; gradient: 35%-65% B over 8.0 min) to give the title compound (Compound 667) (18.8 mg, 22.8 μmol, 4.44% yield, 97.3% purity) as a pale yellow solid. LCMS (ES⁺): m/z 800.4 [M+H]⁺.

### Example 665. Synthesis of trans-4-((5-fluoro-4-(3-(2-oxo-1,3-oxazinan-3-yl)phenyl)pyrimidin-2-yl)amino)cyclohexyl 4-(4-((2,6-dioxopiperidin-3-yl)amino)-2-fluorophenyl)-[1,4′-bipiperidine]-1′-carboxylate (Compound 677)

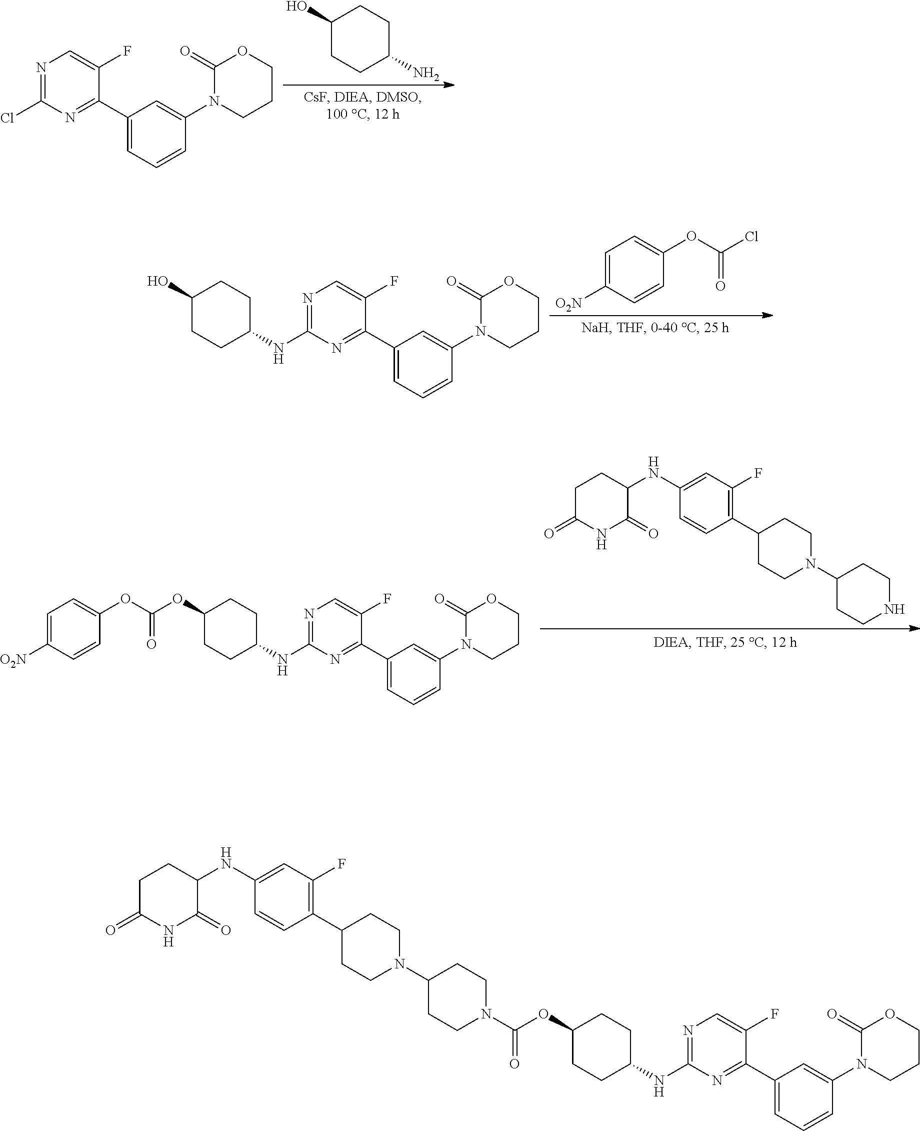

Synthesis of trans-3-(3-(5-fluoro-2-(((1r,4r)-4-hydroxycyclohexyl)amino)pyrimidin-4-yl)phenyl)-1,3-oxazinan-2-one. To a solution of 3-(3-(2-chloro-5-fluoropyrimidin-4-yl)phenyl)-1,3-oxazinan-2-one (11.2 g, 37.1 mmol, 1.00 eq) and trans-4-aminocyclohexan-1-ol (6.40 g, 55.6 mmol, 1.50 eq) in DMSO (500 mL) were added CsF (8.44 g, 55.6 mmol, 1.50 eq) and DIEA (14.4 g, 111 mmol, 19.4 mL, 3.00 eq) at 20 °C. The mixture was stirred at 100 °C for 12 h. LCMS showed 3-(3-(2-chloro-5-fluoropyrimidin-4-yl)phenyl)-1,3-oxazinan-2-one was consumed and the desired target MS was detected. The mixture was filtered and the filter cake was dried under reduced pressure to give the title compound (1.50 g, crude) as a yellow solid. LCMS (ES⁺): m/z 387.1 [M+H]⁺.

Synthesis of trans-4-((5-fluoro-4-(3-(2-oxo-1,3-oxazinan-3-yl)phenyl)pyrimidin-2-yl)amino)cyclohexyl (4-nitrophenyl) carbonate. To a solution of trans-3-(3-(5-fluoro-2-((4-hydroxycyclohexyl)amino)pyrimidin-4-yl)phenyl)-1,3-oxazinan-2-one (900 mg, 2.33 mmol, 1.00 eq) in THE (20 mL) was added NaH (466 mg, 11.7 mmol, 60.0% purity, 3.00 eq) at 0 °C under N₂ atmosphere. The mixture was stirred at 0 °C for 1 h. Then a solution of (4-nitrophenyl) carbonochloridate (1.41 g, 6.99 mmol, 3.00 eq) in THE (10 mL) was added to the mixture at 0 °C. The mixture was slowly warmed to 20 °C and stirred at 40 °C for 24 h under N₂ atmosphere. LCMS showed trans-3-(3-(5-fluoro-2-((4-hydroxycyclohexyl)amino)pyrimidin-4-yl)phenyl)-1,3-oxazinan-2-one was consumed mostly and the desired mass was detected. The reaction mixture was poured into saturated NH₄Cl solution (20 mL) and extracted with EtOAc (20 mL*3). The combined organic layers were dried over anhydrous Na₂SO₄, filtered and the filtrate was concentrated under reduced pressure to give a residue. The residue was purified by MPLC (SiO₂, Petroleum ether:Ethyl acetate=1:0 to 0:1) give the title compound (140 mg, 254 μmol, 10.9% yield) as a yellow oil. LCMS (ES⁺): m/z 552.3 [M+H]⁺.

Synthesis of trans-4-((5-fluoro-4-(3-(2-oxo-1,3-oxazinan-3-yl)phenyl)pyrimidin-2-yl)amino)cyclohexyl 4-(4-((2,6-dioxopiperidin-3-yl)amino)-2-fluorophenyl)-[1,4′-bipiperidine]-1′-carboxylate (Compound 677). To a solution of 3-((4-([1,4′-bipiperidin]-4-yl)-3-fluorophenyl)amino)piperidine-2,6-dione (200 mg, 398 μmol, 1.00 eq, TFA) in THE (4 mL) were added DIEA (257 mg, 1.98 mmol, 347 μL, 5.00 eq) and trans-4-((5-fluoro-4-(3-(2-oxo-1,3-oxazinan-3-yl)phenyl)pyrimidin-2-yl)amino)cyclohexyl (4-nitrophenyl) carbonate (87.8 mg, 159.2 μmol, 0.400 eq) at 25 °C. The mixture was stirred at 25 °C for 12 h. LCMS showed trans-4-((5-fluoro-4-(3-(2-oxo-1,3-oxazinan-3-yl)phenyl)pyrimidin-2-yl)amino)cyclohexyl (4-nitrophenyl) carbonate was consumed completely and the desired mass was detected. The reaction mixture was concentrated under reduced pressure to give a residue. The residue were purified by prep-HPLC (column: Phenomenex luna C18 100*40 mm*3 um; mobile phase: [H₂O (0.2% FA)-ACN]; gradient: 5%-40% B over 8.0 min) to give the title compound (Compound 677) (19.7 mg, 22.5 μmol, 5.66% yield, 96.8% purity, FA) as a white solid. LCMS (ES⁺): m/z 801.4 [M+H]⁺.

### Example 666. Synthesis of trans-4-((5-fluoro-4-(3-(2-oxo-1,3-oxazinan-3-yl)phenyl)pyrimidin-2-yl)amino)cyclohexyl 4-(4-(4-((2,6-dioxopiperidin-3-yl)amino)-2-fluorophenyl)piperazin-1-yl)piperidine-1-carboxylate (Compound 674)

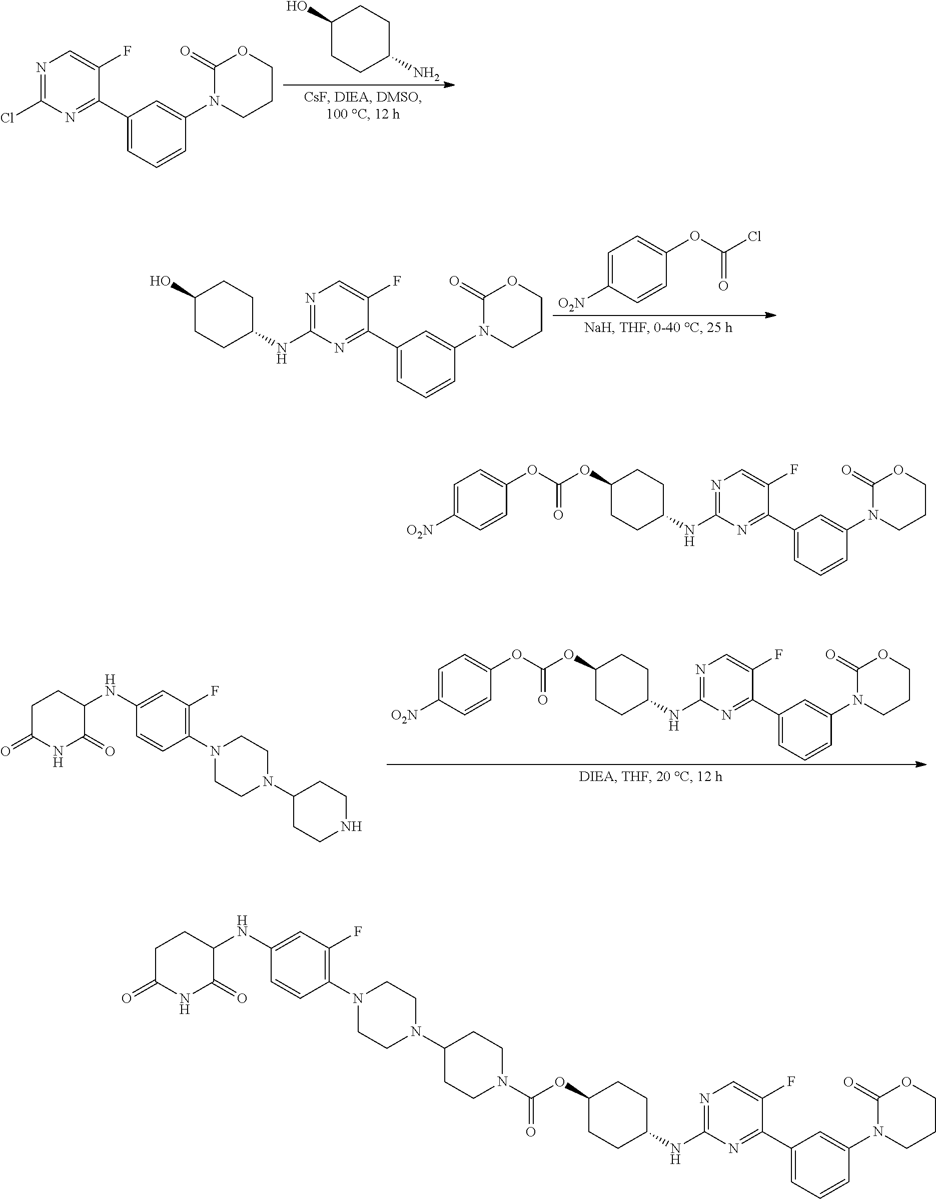

Synthesis of trans-3-(3-(5-fluoro-2-(((1r,4r)-4-hydroxycyclohexyl)amino)pyrimidin-4-yl)phenyl)-1,3-oxazinan-2-one. To a solution of 3-(3-(2-chloro-5-fluoropyrimidin-4-yl)phenyl)-1,3-oxazinan-2-one (11.2 g, 37.1 mmol, 1.00 eq) and trans-4-aminocyclohexan-1-ol (6.40 g, 55.6 mmol, 1.50 eq) in DMSO (500 mL) were added CsF (8.44 g, 55.6 mmol, 1.50 eq) and DIEA (14.4 g, 111 mmol, 19.4 mL, 3.00 eq) at 20 °C. The mixture was stirred at 100 °C for 12 h. LCMS showed 3-(3-(2-chloro-5-fluoropyrimidin-4-yl)phenyl)-1,3-oxazinan-2-one was consumed and the desired target MS was detected. The mixture was filtered and the filter cake was dried under reduced pressure to give the title compound (1.50 g, crude) as a yellow solid. LCMS (ES⁺): m/z 387.1 [M+H]⁺.

Synthesis of trans-4-((5-fluoro-4-(3-(2-oxo-1,3-oxazinan-3-yl)phenyl)pyrimidin-2-yl)amino)cyclohexyl (4-nitrophenyl) carbonate. To a solution of trans-3-(3-(5-fluoro-2-((4-hydroxycyclohexyl)amino)pyrimidin-4-yl)phenyl)-1,3-oxazinan-2-one (900 mg, 2.33 mmol, 1.00 eq) in THF (20 mL) was added NaH (466 mg, 11.7 mmol, 60.0% purity, 3.00 eq) at 0 °C under N₂ atmosphere. The mixture was stirred at 0 °C for 1 h. Then a solution of (4-nitrophenyl) carbonochloridate (1.41 g, 6.99 mmol, 3.00 eq) in THF (10 mL) was added to the mixture at 0 °C. The mixture was slowly warmed to 20 °C and stirred at 40 °C for 24 h under N₂ atmosphere. LCMS showed trans-3-(3-(5-fluoro-2-((4-hydroxycyclohexyl)amino)pyrimidin-4-yl)phenyl)-1,3-oxazinan-2-one was consumed mostly and the desired mass was detected. The reaction mixture was poured into saturated NH₄Cl solution (20 mL) and extracted with EtOAc (20 mL*3). The combined organic layers were dried over anhydrous Na₂SO₄, filtered and the filtrate was concentrated under reduced pressure to give a residue. The residue was purified by MPLC (SiO₂, Petroleum ether:Ethyl acetate=1:0 to 0:1) give the title compound (140 mg, 254 μmol, 10.9% yield) as a yellow oil. LCMS (ES⁺): m/z 552.3 [M+H]⁺.

Synthesis of trans-4-((5-fluoro-4-(3-(2-oxo-1,3-oxazinan-3-yl)phenyl)pyrimidin-2-yl)amino)cyclohexyl 4-(4-(4-((2,6-dioxopiperidin-3-yl)amino)-2-fluorophenyl)piperazin-1-yl)piperidine-1-carboxylate (Compound 674). To a solution of 3-((3-fluoro-4-(4-(piperidin-4-yl)piperazin-1-yl)phenyl)amino)piperidine-2,6-dione (135 mg, 268 μmol, 1.00 eq, TFA) and trans-4-((5-fluoro-4-(3-(2-oxo-1,3-oxazinan-3-yl)phenyl)pyrimidin-2-yl)amino)cyclohexyl (4-nitrophenyl) carbonate (59.2 mg, 107 μmol, 0.400 eq) in THF (4 mL) was added DIEA (177 mg, 1.37 mmol, 238 μL, 5.00 eq) at 20 °C. The mixture was stirred at 20 °C for 12 h. LCMS showed 3-((3-fluoro-4-(4-(piperidin-4-yl)piperazin-1-yl)phenyl)amino)piperidine-2,6-dione was consumed and the desired target MS was detected. The mixture was concentrated under reduced pressure to give a residue. The residue was purified by prep-HPLC (column: Phenomenex Luna C18 100*30 mm*3 um; mobile phase: [H₂O (0.2% FA)-ACN]; gradient: 15%-50% B over 8.0 min) to give the title compound (Compound 674) (26.1 mg, 29.9 μmol, 11.2% yield, 97.2% purity, FA) as a pale yellow solid. LCMS (ES⁺): m/z 802.3 [M+H]⁺.

### Example 667. Synthesis of trans-3-[3-fluoro-4-[4-[[4-[4-[[5-fluoro-4-[3-(3-oxomorpholin-4-yl)phenyl]pyrimidin-2-yl]amino]cyclohexanecarbonyl]cyclohexyl]methyl]piperazin-1-yl]anilino]piperidine-2,6-dione (Compound 683)

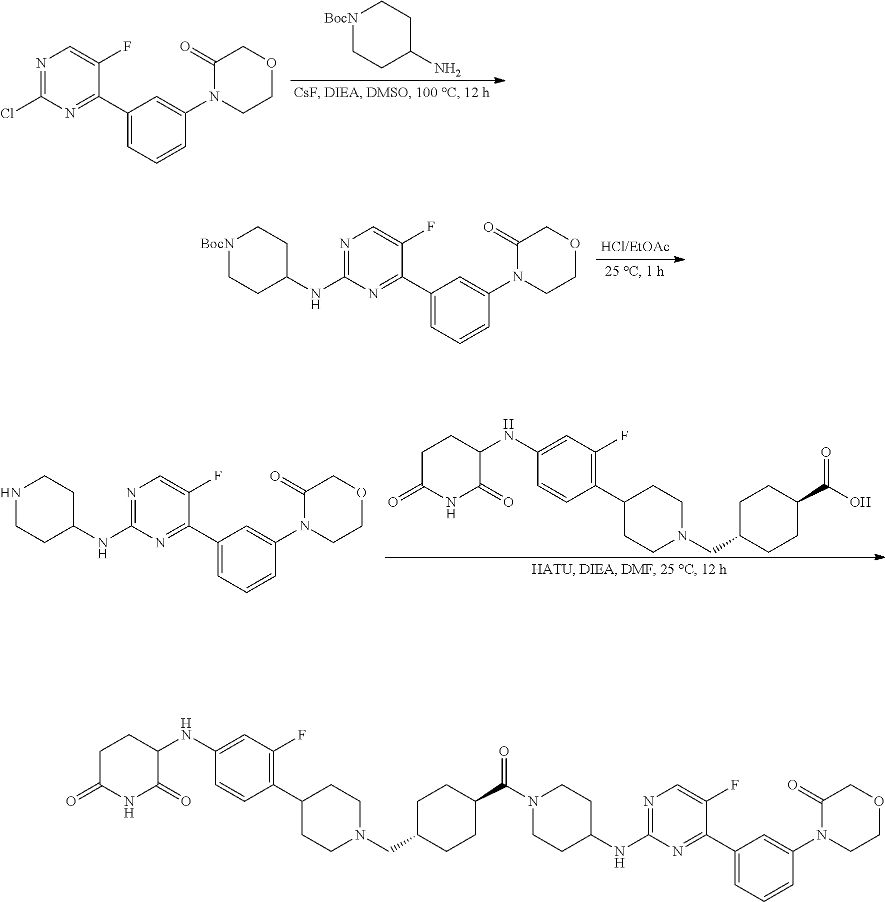

Synthesis of tert-butyl 4-((5-fluoro-4-(3-(3-oxomorpholino)phenyl)pyrimidin-2-yl)amino)piperidine-1-carboxylate. To a solution of 4-[3-(2-chloro-5-fluoro-pyrimidin-4-yl)phenyl]morpholin-3-one (3.00 g, 9.75 mmol, 1.00 eq) and tert-butyl 4-aminopiperidine-1-carboxylate (1.95 g, 9.75 mmol, 1.00 eq) in DMSO (15 mL) was added CsF (2.96 g, 19.5 mmol, 719 μL, 2.00 eq) and DIEA (1.26 g, 9.75 mmol, 1.70 mL, 1.00 eq) at 25 °C. The mixture was stirred at 100 °C for 12 h. LC-MS showed the desired mass was detected. The mixture was diluted with water (60 mL), and extracted with EtOAc (50 mL*3). The combined organic layers were washed with brine (20 mL), dried over Na₂SO₄, filtered and concentrated under reduced pressure. The residue was purified by column chromatography (SiO₂, Petroleum ether:Ethyl acetate=2:1 to 1:1) to give the title compound (3.20 g, 6.11 mmol, 62.6% yield, 90.0% purity) as a white solid. LCMS (ES⁺): m/z 372.3 [M−100+H]⁺.

Synthesis of 4-(3-(5-fluoro-2-(piperidin-4-ylamino)pyrimidin-4-yl)phenyl)morpholin-3-one. A solution of tert-butyl 4-[[5-fluoro-4-[3-(3-oxomorpholin-4-yl)phenyl]pyrimidin-2-yl]amino]piperidine-1-carboxylate (450 mg, 954 μmol, 1.00 eq) in HCl/EtOAc (4.00 M, 5 mL, 20.9 eq) was stirred at 25 °C for 1 h. LC-MS showed the desired mass was detected. The reaction mixture was concentrated under reduced pressure to give the title compound (350 mg, crude, HCl) as a white solid. LCMS (ES⁺): m/z 372.2 [M+H]⁺.

Synthesis of 3-((3-fluoro-4-(1-(((trans)-4-(4-((5-fluoro-4-(3-(3-oxomorpholino)phenyl)pyrimidin-2-yl)amino)piperidine-1-carbonyl)cyclohexyl)methyl)piperidin-4-yl)phenyl)amino)piperidine-2,6-dione (Compound 683). To a solution of trans-4-[[4-[4-[(2,6-dioxo-3-piperidyl)amino]-2-fluoro-phenyl]-1-piperidyl]methyl]cyclohexanecarboxylic acid (99.8 mg, 178 μmol, 1.00 eq, TFA) in DMF (1 mL) was added 4-[3-[5-fluoro-2-(4-piperidylamino)pyrimidin-4-yl]phenyl]morpholin-3-one (66.2 mg, 162 μmol, 0.911 eq, HCl) was added DIEA (230 mg, 1.78 mmol, 310.0 μL, 10.0 eq) and HATU (67.8 mg, 178 μmol, 1.00 eq) at 25 °C. The mixture was stirred at 25 °C for 12 h. LC-MS showed the desired mass was detected. The mixture was purified by prep-HPLC (column: Phenomenex Luna C18 100*30 mm*3 um; mobile phase: [H₂O (0.2% FA)-ACN]; gradient: 5%-35% B over 8.0 min). The residue was purified by prep-HPLC (column: Phenomenex luna C18 100*40 mm*5 um; mobile phase: [H₂O (0.04% HCl)-ACN]; gradient: 5%-30% B over 8.0 min) to give the title compound (Compound 683) (43.0 mg, 51.7 μmol, 29.0% yield, 96.2% purity) as a green gum. LCMS (ES⁺): m/z 799.4 [M+H]⁺.

### Example 668. Synthesis of trans-4-((5-fluoro-4-(3-(3-oxomorpholino)phenyl)pyrimidin-2-yl)amino)cyclohexyl 4-(4-((2,6-dioxopiperidin-3-yl)amino)-2-fluorophenyl)-[1,4′-bipiperidine]-1′-carboxylate (Compound 690)

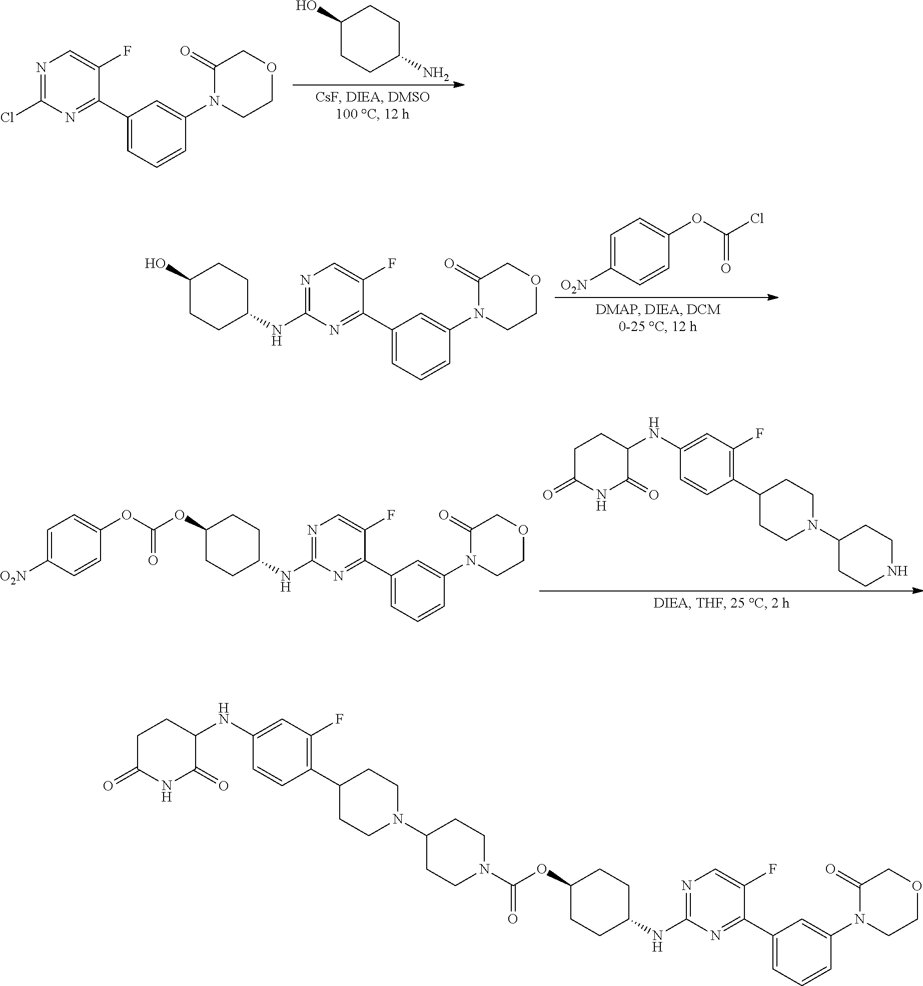

Synthesis of trans-4-(3-(5-fluoro-2-((4-hydroxycyclohexyl)amino)pyrimidin-4-yl)phenyl)morpholin-3-one. To a solution of 4-[3-(2-chloro-5-fluoro-pyrimidin-4-yl)phenyl]morpholin-3-one (4.00 g, 13.0 mmol, 1.00 eq) and trans-4-aminocyclohexanol (2.25 g, 19.5 mmol, 1.50 eq) in DMSO (150 mL) were added CsF (2.96 g, 19.5 mmol, 1.50 eq) and DIEA (5.04 g, 39.0 mmol, 6.79 mL, 3.00 eq) at 25 °C. The mixture was stirred at 100 °C for 12 h. LCMS showed 4-[3-(2-chloro-5-fluoro-pyrimidin-4-yl)phenyl]morpholin-3-one was consumed completely and the desired mass was detected. Water (30 mL) was added to the mixture at 0 °C. The reaction mixture was filtered and the filter cake was dried in vacuum to give the title compound (4.00 g, crude) as a pale yellow solid. LCMS (ES⁺): m/z 387.1 [M+H]⁺.

Synthesis of trans-4-((5-fluoro-4-(3-(3-oxomorpholino)phenyl)pyrimidin-2-yl)amino)cyclohexyl (4-nitrophenyl) carbonate. To a solution of trans-4-[3-[5-fluoro-2-[(4-hydroxycyclohexyl)amino]pyrimidin-4-yl]phenyl]morpholin-3-one (3.60 g, 9.32 mmol, 1.00 eq) in DCM (130 mL) was added (4-nitrophenyl) carbonochloridate (4.69 g, 23.3 mmol, 2.50 eq) at 25 °C. Then DMAP (1.14 g, 9.32 mmol, 1.00 eq) and DIEA (3.61 g, 28.0 mmol, 4.87 mL, 3.00 eq) were added to the mixture at 0 °C. The mixture was stirred at 25 °C for 12 hr. LCMS showed trans-4-[3-[5-fluoro-2-[(4-hydroxycyclohexyl)amino]pyrimidin-4-yl]phenyl]morpholin-3-one was consumed completely and the desired mass was detected. The reaction mixture filtered and the filtrate was concentrated under reduced pressure to give a residue. Water (40 mL) and EtOAc (40 mL) was added to the residue at 25 °C. The reaction mixture was filtered and the filter cake was dried in vacuum to give the title compound (4.00 g, crude) as a pale yellow solid. LCMS (ES⁺): m/z 552.2 [M+H]⁺.

Synthesis of trans-4-((5-fluoro-4-(3-(3-oxomorpholino)phenyl)pyrimidin-2-yl)amino)cyclohexyl 4-(4-((2,6-dioxopiperidin-3-yl)amino)-2-fluorophenyl)-[1,4′-bipiperidine]-1′-carboxylate (Compound 690). To a solution of 3-((4-([1,4′-bipiperidin]-4-yl)-3-fluorophenyl)amino)piperidine-2,6-dione (200 mg, 398 μmol, 1.00 eq, TFA) in THF (3 mL) were added DIEA (154 mg, 1.19 mmol, 208 μL, 3.00 eq) and trans-[4-[[5-fluoro-4-[3-(3-oxomorpholin-4-yl)phenyl]pyrimidin-2-yl]amino]cyclohexyl](4-nitrophenyl) carbonate (87.8 mg, 159 μmol, 0.400 eq) at 25 °C. The mixture was stirred at 25 °C for 2 h. LCMS showed trans-[4-[[5-fluoro-4-[3-(3-oxomorpholin-4-yl)phenyl]pyrimidin-2-yl]amino]cyclohexyl](4-nitrophenyl) carbonate was consumed completely and the desired mass was detected. The reaction mixture was concentrated under reduced pressure to give a residue. The residue was purified by prep-HPLC (column: Phenomenex Luna C18 75*30 mm*3 um; mobile phase: [water (0.04% HCl)-ACN]; gradient: 10%-40% B over 8.0 min) to give the title compound (Compound 690) (64.6 mg, 77.8 μmol, 19.6% yield, 96.5% purity) as a yellow solid. LCMS (ES⁺): m/z 801.4 [M+H]⁺.

### Example 669. Synthesis of trans-4-((5-fluoro-4-(3-(3-oxomorpholino)phenyl)pyrimidin-2-yl)amino)cyclohexyl 4-(4-(4-((2,6-dioxopiperidin-3-yl)amino)-2-fluorophenyl)piperazin-1-yl)piperidine-1-carboxylate (Compound 688)

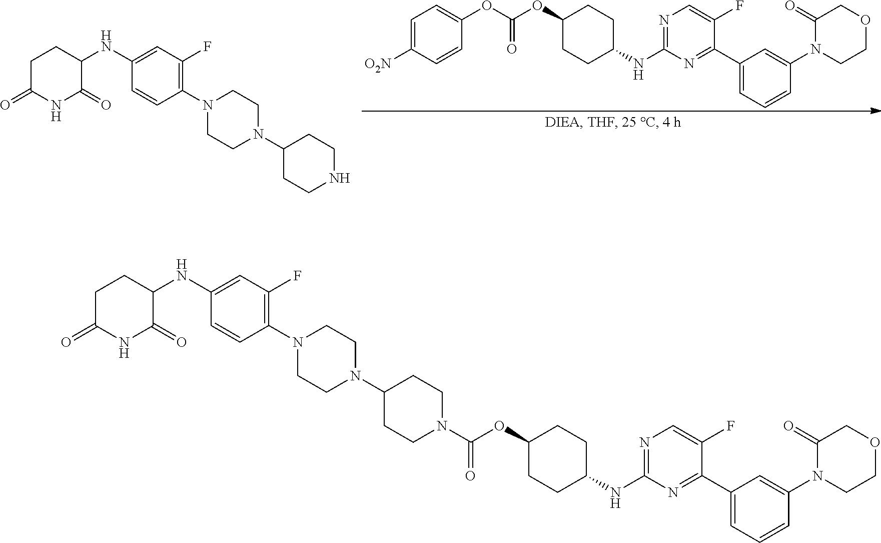 [SMILES: O=C1CCC(Nc2ccc(N3CCN(C4CCNCC4)CC3)c(F)c2)C(=O)N1 ; O=C(Oc1ccc([N+](=O)[O-])cc1)O[C@H]1CC[C@H](Nc2ncc(F)c(-c3cccc(N4CCOCC4=O)c3)n2)CC1 ; O=C1CCC(Nc2ccc(N3CCN(C4CCN(C(=O)O[C@H]5CC[C@H](Nc6ncc(F)c(-c7cccc(N8CCOCC8=O)c7)n6)CC5)CC4)CC3)c(F)c2)C(=O)N1]

Synthesis of trans-4-((5-fluoro-4-(3-(3-oxomorpholino)phenyl)pyrimidin-2-yl)amino)cyclohexyl 4-(4-(4-((2,6-dioxopiperidin-3-yl)amino)-2-fluorophenyl)piperazin-1-yl)piperidine-1-carboxylate (Compound 688). To a solution of 3-((3-fluoro-4-(4-(piperidin-4-yl)piperazin-1-yl)phenyl)amino)piperidine-2,6-dione (200 mg, 397 μmol, 1.00 eq, TFA) in THF (4 mL) were added DIEA (258 mg, 1.99 mmol, 345 μL, 5.00 eq) and trans-4-((5-fluoro-4-(3-(3-oxomorpholino)phenyl)pyrimidin-2-yl)amino)cyclohexyl (4-nitrophenyl) carbonate (131 mg, 238 μmol, 0.600 eq) at 25 °C, the resulting mixture was stirred at 25 °C for 4 h. LCMS showed 3-((3-fluoro-4-(4-(piperidin-4-yl)piperazin-1-yl)phenyl)amino)piperidine-2,6-dione was consumed completely and the desired mass was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 20%-50% B over 8.0 min) to give the title compound (Compound 688) (83.1 mg, 100 μmol, 25.2% yield, 96.7% purity) as a white solid. LCMS (ES⁺): m/z 802.3 [M+H]⁺.

### Example 670. Synthesis of trans-4-((5-fluoro-4-(3-morpholinophenyl)pyrimidin-2-yl)amino)cyclohexyl 4-(4-((2,6-dioxopiperidin-3-yl)amino)-2-fluorophenyl)-[1,4′-bipiperidine]-1′-carboxylate (Compound 676)

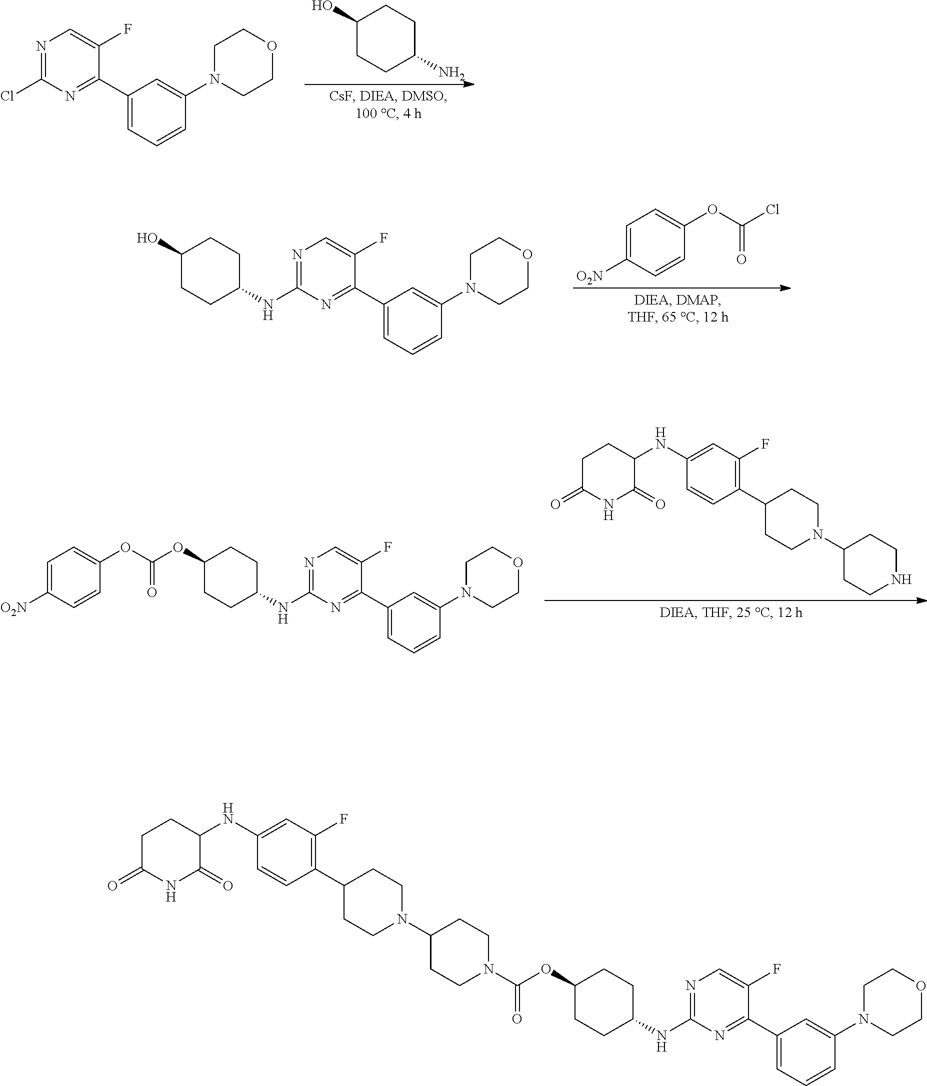

Synthesis of trans-4-((5-fluoro-4-(3-morpholinophenyl)pyrimidin-2-yl)amino)cyclohexan-1-ol. To a solution of 4-(3-(2-chloro-5-fluoropyrimidin-4-yl)phenyl)morpholine (3.40 g, 11.6 mmol, 1.00 eq) and trans-4-aminocyclohexan-1-ol (2.00 g, 17.4 mmol, 1.50 eq) in DMSO (80 mL) were added CsF (2.64 g, 17.4 mmol, 1.50 eq) and DIEA (4.49 g, 34.7 mmol, 6.05 mL, 3.00 eq) at 25 °C, the mixture was stirred at 100 °C for 4 h. LCMS showed that 4-(3-(2-chloro-5-fluoropyrimidin-4-yl)phenyl)morpholine was consumed and the desired mass was detected. H₂O (80 mL) was added to the mixture and the mixture was extracted with EtOAc (70 mL*3). The combined organic layers were washed with brine (50 mL*3), dried over Na₂SO₄, filtered and the filtrate was concentrated under reduced pressure to give the title compound (6.00 g, crude) as a yellow solid. LCMS (ES⁺): m/z 373.2 [M+H]⁺.

Synthesis of trans-4-((5-fluoro-4-(3-morpholinophenyl)pyrimidin-2-yl)amino)cyclohexyl (4-nitrophenyl) carbonate. To a solution of trans-4-((5-fluoro-4-(3-morpholinophenyl)pyrimidin-2-yl)amino)cyclohexan-1-ol (3.50 g, 9.40 mmol, 1.00 eq) and (4-nitrophenyl) carbonochloridate (11.4 g, 56.4 mmol, 6.00 eq) in THF (200 mL) were added DIEA (2.43 g, 18.8 mmol, 3.27 mL, 2.00 eq) and DMAP (1.15 g, 9.40 mmol, 1.00 eq) at 25 °C, the mixture was stirred at 65 °C for 12 h. LCMS showed that trans-4-((5-fluoro-4-(3-morpholinophenyl)pyrimidin-2-yl)amino)cyclohexan-1-ol was consumed and the desired mass was detected. H₂O (100 mL) was added to the mixture and the mixture was extracted with EtOAc (150 mL*3). The combined organic layers were dried over Na₂SO₄, filtered and the filtrate was concentrated under reduced pressure to give a residue. The residue was purified by MPLC (SiO₂, Petroleum ether:Ethyl acetate=1:0 to 3:1) to give the title compound (1.00 g, 1.86 mmol, 19.8% yield) as a yellow solid. LCMS (ES⁺): m/z 538.3 [M+H]⁺.

Synthesis of trans-4-((5-fluoro-4-(3-morpholinophenyl)pyrimidin-2-yl)amino)cyclohexyl 4-(4-((2,6-dioxopiperidin-3-yl)amino)-2-fluorophenyl)-[1,4′-bipiperidine]-1′-carboxylate (Compound 676). To a solution of 3-[3-fluoro-4-[1-(4-piperidyl)-4-piperidyl]anilino]piperidine-2,6-dione (200 mg, 398 μmol, 1.00 eq, TFA) in THF (7 mL) were added DIEA (154 mg, 1.19 mmol, 208 μL, 3.00 eq) and trans-4-((5-fluoro-4-(3-morpholinophenyl)pyrimidin-2-yl)amino)cyclohexyl (4-nitrophenyl) carbonate (214 mg, 398 μmol, 1.00 eq) at 25 °C. The mixture was stirred at 25 °C for 12 h. LCMS showed trans-4-((5-fluoro-4-(3-morpholinophenyl)pyrimidin-2-yl)amino)cyclohexyl (4-nitrophenyl) carbonate was consumed completely and the desired mass was detected. The reaction mixture was concentrated under reduced pressure to give a residue. The residue were purified by prep-HPLC (column: Phenomenex Luna C18 100*30 mm*3 um; mobile phase: [H₂O (0.2% FA)-ACN]; gradient: 20%-50% B over 8.0 min) to give the title compound (Compound 676) (6.90 mg, 8.28 μmol, 2.08% yield, 100% purity, FA) as a white solid. LCMS (ES⁺): m/z 787.5 [M+H]⁺.

### Example 671. Synthesis of trans-4-((5-fluoro-4-(3-morpholinophenyl)pyrimidin-2-yl)amino)cyclohexyl 4-(4-(4-((2,6-dioxopiperidin-3-yl)amino)-2-fluorophenyl)piperazin-1-yl)piperidine-1-carboxylate (Compound 673)

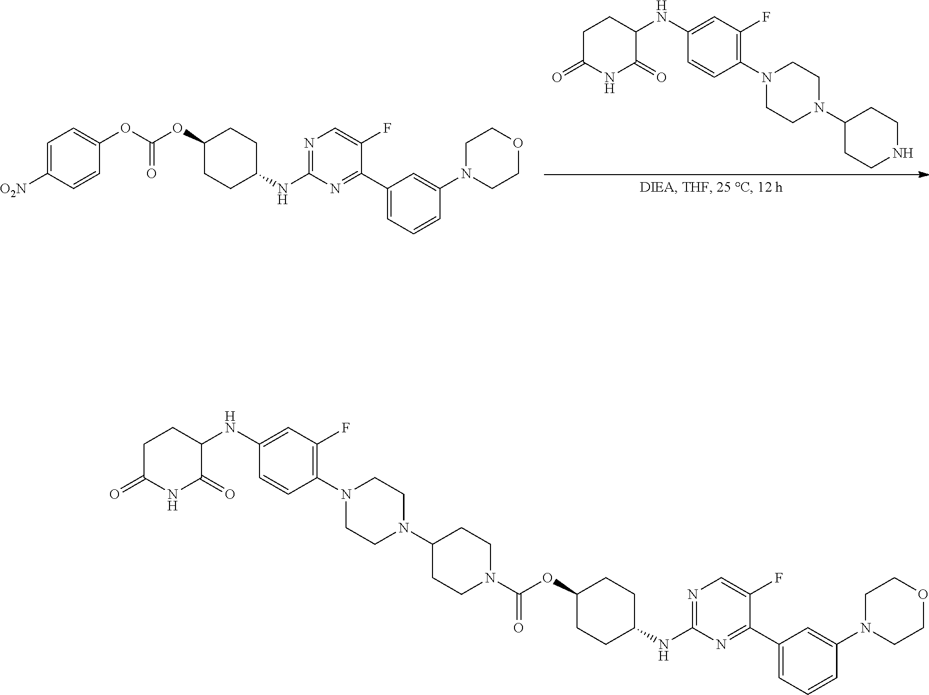 [SMILES: O=C(Oc1ccc([N+](=O)[O-])cc1)O[C@H]1CC[C@H](Nc2ncc(F)c(-c3cccc(N4CCOCC4)c3)n2)CC1 ; O=C1CCC(Nc2ccc(N3CCN(C4CCNCC4)CC3)c(F)c2)C(=O)N1 ; O=C1CCC(Nc2ccc(N3CCN(C4CCN(C(=O)O[C@H]5CC[C@H](Nc6ncc(F)c(-c7cccc(N8CCOCC8)c7)n6)CC5)CC4)CC3)c(F)c2)C(=O)N1]

Synthesis of trans-4-((5-fluoro-4-(3-morpholinophenyl)pyrimidin-2-yl)amino)cyclohexyl 4-(4-(4-((2,6-dioxopiperidin-3-yl)amino)-2-fluorophenyl)piperazin-1-yl)piperidine-1-carboxylate (Compound 673). To a solution of trans-4-((5-fluoro-4-(3-morpholinophenyl)pyrimidin-2-yl)amino)cyclohexyl (4-nitrophenyl) carbonate (100 mg, 199 mol, 1.00 eq, TFA) in THF (4 mL) were added DIEA (77.1 mg, 597 μmol, 104 μL, 3.00 eq) and 3-((3-fluoro-4-(4-(piperidin-4-yl)piperazin-1-yl)phenyl)amino)piperidine-2,6-dione (107 mg, 199 μmol, 1.00 eq) was added at 25 °C, the mixture was stirred at 25 °C for 12 h. LCMS showed that trans-4-((5-fluoro-4-(3-morpholinophenyl)pyrimidin-2-yl)amino)cyclohexyl (4-nitrophenyl) carbonate was consumed and the desired mass was detected. The mixture was concentrated under reduced pressure to give a residue. This residue was purified by prep-HPLC (column: Phenomenex luna C18 100*40 mm*3 um; mobile phase: [H₂O (0.2% FA)-ACN]; gradient: 20%-60% B over 8.0 min) to give the title compound (Compound 673) (18.0 mg, 22.6 μmol, 11.40% yield, 99.07% purity) as a white solid LCMS (ES⁺): m/z 788.4 [M+H]⁺.

### Example 672. Synthesis of trans-3-((3-fluoro-4-(1-((1-(4-((5-fluoro-4-(3-morpholinophenyl)pyrimidin-2-yl)amino)cyclohexane-1-carbonyl)piperidin-4-yl)methyl)piperidin-4-yl)phenyl)amino)piperidine-2,6-dione (Compound 672)

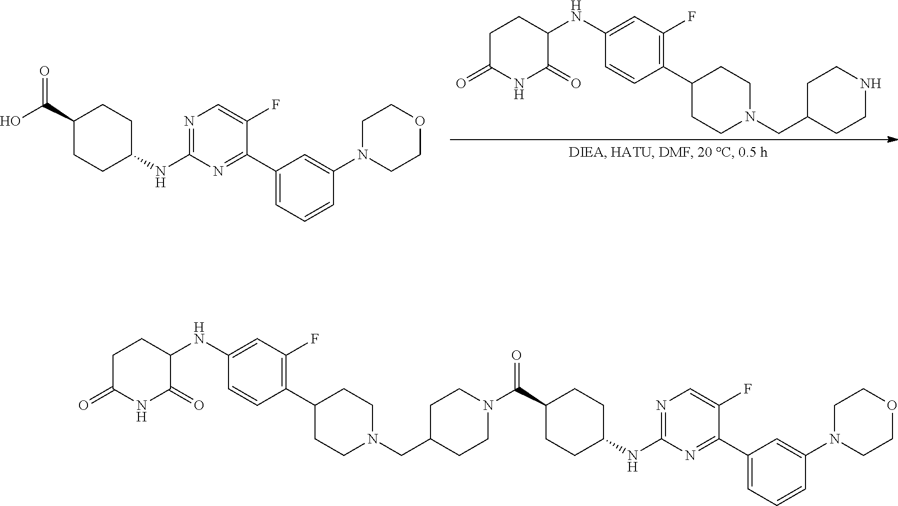 [SMILES: O=C(O)[C@H]1CC[C@H](Nc2ncc(F)c(-c3cccc(N4CCOCC4)c3)n2)CC1 ; O=C1CCC(Nc2ccc(C3CCN(CC4CCNCC4)CC3)c(F)c2)C(=O)N1 ; O=C1CCC(Nc2ccc(C3CCN(CC4CCN(C(=O)[C@H]5CC[C@H](Nc6ncc(F)c(-c7cccc(N8CCOCC8)c7)n6)CC5)CC4)CC3)c(F)c2)C(=O)N1]

Synthesis of trans-3-((3-fluoro-4-(1-((1-(4-((5-fluoro-4-(3-morpholinophenyl)pyrimidin-2-yl)amino)cyclohexane-1-carbonyl)piperidin-4-yl)methyl)piperidin-4-yl)phenyl)amino)piperidine-2,6-dione (Compound 672). To a solution of trans-4-((5-fluoro-4-(3-morpholinophenyl)pyrimidin-2-yl)amino)cyclohexane-1-carboxylic acid (109 mg, 271 μmol, 0.700 eq) in DMF (3 mL) were added HATU (58.9 mg, 155 μmol, 0.400 eq) and DIEA (250 mg, 1.94 mmol, 338 μL, 5.00 eq) at 20 °C, then to the mixture was added 3-((3-fluoro-4-(1-(piperidin-4-ylmethyl)piperidin-4-yl)phenyl)amino)piperidine-2,6-dione (200 mg, 387 μmol, 1.00 eq, TFA) at 20 °C, the mixture was stirred at 20 °C for 0.5 h. LCMS showed trans-4-((5-fluoro-4-(3-morpholinophenyl)pyrimidin-2-yl)amino)cyclohexane-1-carboxylic acid was consumed and the desired mass was detected. The mixture was concentrated under reduced pressure to give a residue. The residue was purified by prep-HPLC (column: 2_Phenomenex Gemini C18 75*40 mm*3 um; mobile phase: [H₂O (10 mM NH₄HCO₃)-ACN]; gradient: 45%-75% B over 8.0 min) to give the title compound (Compound 672) (23.8 mg, 28.5 μmol, 7.35% yield, 93.9% purity) as a white solid. LCMS (ES⁺): m/z 785.5 [M+H]⁺.

### Example 673. Synthesis of trans-3-((3-fluoro-4-(4-((1-(4-((5-fluoro-4-(3-morpholinophenyl)pyrimidin-2-yl)amino)cyclohexane-1-carbonyl)piperidin-4-yl)methyl)piperazin-1-yl)phenyl)amino)piperidine-2,6-dione (Compound 686)

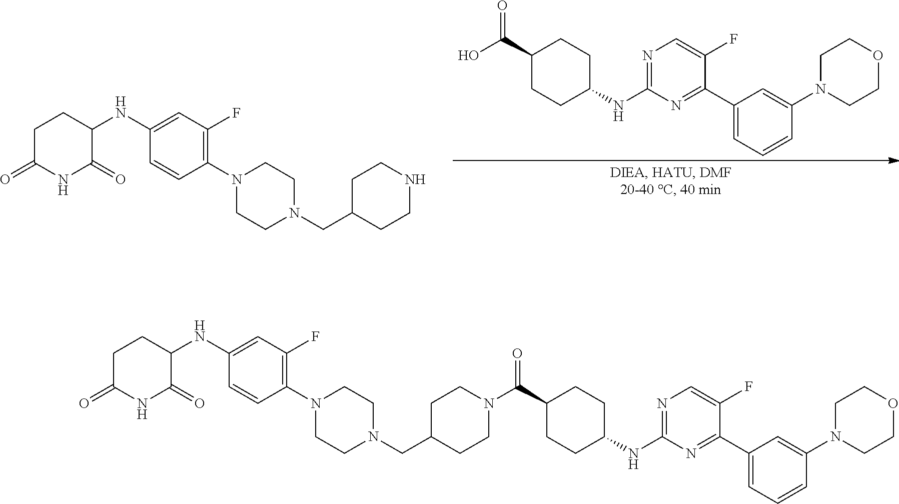 [SMILES: O=C1CCC(Nc2ccc(N3CCN(CC4CCNCC4)CC3)c(F)c2)C(=O)N1 ; O=C(O)[C@H]1CC[C@H](Nc2ncc(F)c(-c3cccc(N4CCOCC4)c3)n2)CC1 ; O=C1CCC(Nc2ccc(N3CCN(CC4CCN(C(=O)[C@H]5CC[C@H](Nc6ncc(F)c(-c7cccc(N8CCOCC8)c7)n6)CC5)CC4)CC3)c(F)c2)C(=O)N1]

Synthesis of trans-3-((3-fluoro-4-(4-((1-(4-((5-fluoro-4-(3-morpholinophenyl)pyrimidin-2-yl)amino)cyclohexane-1-carbonyl)piperidin-4-yl)methyl)piperazin-1-yl)phenyl)amino)piperidine-2,6-dione (Compound 686). To a solution of trans-4-((5-fluoro-4-(3-morpholinophenyl)pyrimidin-2-yl)amino)cyclohexane-1-carboxylic acid (108 mg, 271 μmol, 1.00 eq) in DMF (4 mL) were added DIEA (69.9 mg, 541 μmol, 94.2 μL, 2.00 eq) and HATU (103 mg, 271 μmol, 1.00 eq) at 25 °C, the mixture was stirred at 20 °C for 10 min. Then 3-((3-fluoro-4-(4-(piperidin-4-ylmethyl)piperazin-1-yl)phenyl)amino)piperidine-2,6-dione (140 mg, 271 μmol, 1.00 eq, TFA) was added at 20 °C, the mixture was stirred at 40 °C for 30 min. LCMS showed that 3-((3-fluoro-4-(4-(piperidin-4-ylmethyl)piperazin-1-yl)phenyl)amino)piperidine-2,6-dione was consumed and the desired mass was detected. To the mixture was added H₂O (5 mL) and the mixture was extracted with EtOAc (5 mL*3). The combined organic layers were washed with saturated aqueous NaCl solution (5 mL*3), dried over Na₂SO₄, filtered and the filtrate was concentrated under reduced pressure to give a residue. The residue was purified by prep-HPLC (column: Phenomenex Luna C18 100*30 mm*3 um; mobile phase: [H₂O (0.2% FA)-ACN]; gradient: 10%-40% B over 8.0 min) to give the title compound (Compound 686) (68.4 mg, 82.2 μmol, 30.39% yield, 100% purity, FA) as a pale yellow solid. LCMS (ES⁺): m/z 786.4 [M+H]⁺.

### Example 674. Synthesis of 3-((3-fluoro-4-(1-((1-((trans)-4-((5-fluoro-4-(3-morpholinophenyl)pyrimidin-2-yl)amino)cyclohexane-1-carbonyl)-4-hydroxypiperidin-4-yl)methyl)piperidin-4-yl)phenyl)amino)piperidine-2,6-dione (Compound 665)

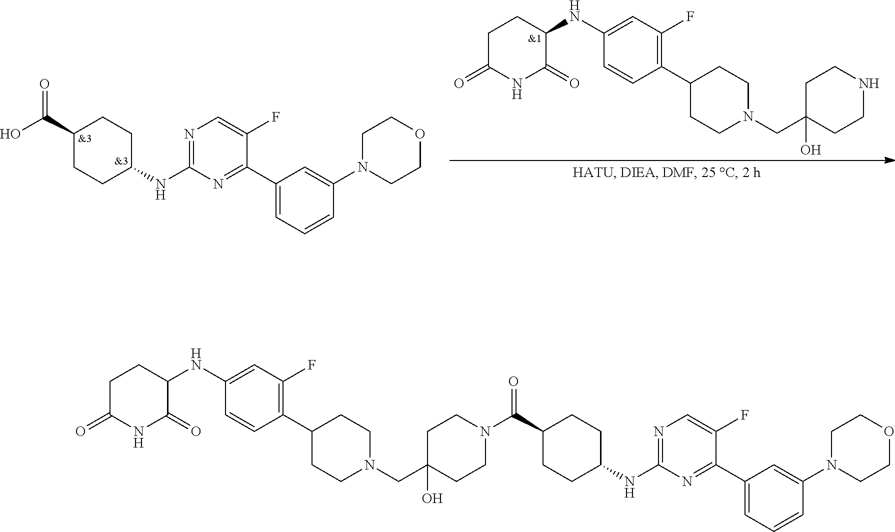 [SMILES: O=C(O)[C@H]1CC[C@H](Nc2ncc(F)c(-c3cccc(N4CCOCC4)c3)n2)CC1 ; O=C1CC[C@@H](Nc2ccc(C3CCN(CC4(O)CCNCC4)CC3)c(F)c2)C(=O)N1 ; O=C1CCC(Nc2ccc(C3CCN(CC4(O)CCN(C(=O)[C@H]5CC[C@H](Nc6ncc(F)c(-c7cccc(N8CCOCC8)c7)n6)CC5)CC4)CC3)c(F)c2)C(=O)N1]

Synthesis of trans-3-[3-fluoro-4-[1-[[1-[4-[[5-fluoro-4-(3-morpholinophenyl)pyrimidin-2-yl]amino]cyclohexanecarbonyl]-4-hydroxy-4-piperidyl]methyl]-4-piperidyl]anilino]piperidine-2,6-dione (Compound 665). To a solution of 4-[[5-fluoro-4-(3-morpholinophenyl)pyrimidin-2-yl]amino]cyclohexanecarboxylic acid (100 mg, 249.72 μmol, 1.00 eq) and 3-[3-fluoro-4-[1-[(4-hydroxy-4-piperidyl)methyl]-4-piperidyl]anilino]piperidine-2,6-dione (104 mg, 249 μmol, 1.00 eq) in DMF (3 mL) was added HATU (142 mg, 374 μmol, 1.50 eq) and DIEA (322 mg, 2.50 mmol, 434 μL, 10.0 eq) at 25 °C. The mixture was stirred at 25 °C for 2 h. LC-MS showed 4-[[5-fluoro-4-(3-morpholinophenyl)pyrimidin-2-yl]amino]cyclohexanecarboxylic acid was consumed completely and the desired mass was detected. The reaction mixture was concentrated under reduced pressure. The residue was purified by prep-HPLC (TFA condition; or neutral condition; or basic condition) to give the title compound (Compound 665) (46.1 mg, 55.88 μmol, 22.38% yield, 97.637% purity, 0.1 FA) as a pale yellow solid. LCMS (ES⁺): m/z 801.4 [M+H]⁺.

### Example 675. Synthesis of trans-(1r,4R)—N-((1r,4R)-4-(4-(4-((2,6-dioxopiperidin-3-yl)amino)-2-fluorophenyl)piperazin-1-yl)cyclohexyl)-4-((5-fluoro-4-(3-morpholinophenyl)pyrimidin-2-yl)amino)cyclohexane-1-carboxamide (Compound 661)

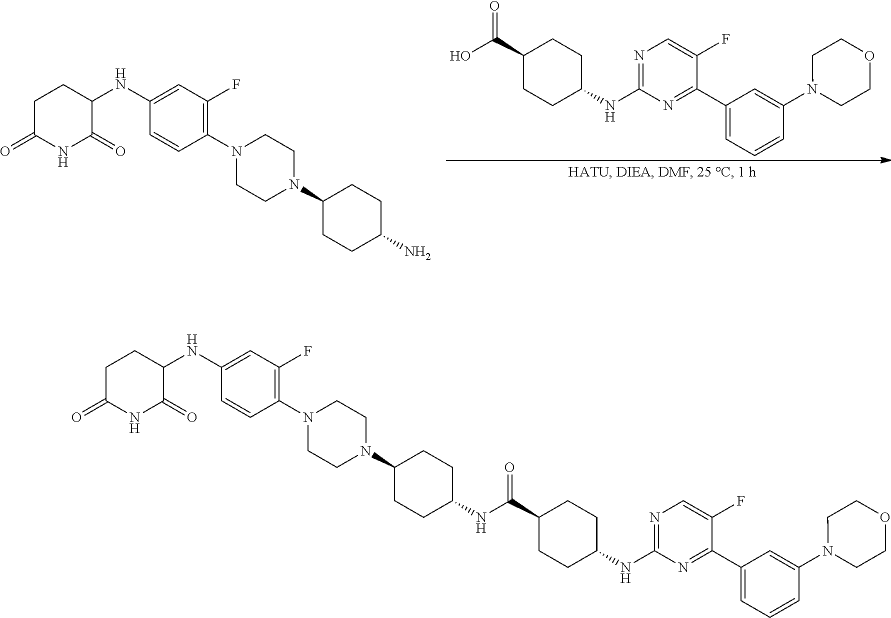 [SMILES: N[C@H]1CC[C@H](N2CCN(c3ccc(NC4CCC(=O)NC4=O)cc3F)CC2)CC1 ; O=C(O)[C@H]1CC[C@H](Nc2ncc(F)c(-c3cccc(N4CCOCC4)c3)n2)CC1 ; O=C1CCC(Nc2ccc(N3CCN([C@H]4CC[C@H](NC(=O)[C@H]5CC[C@H](Nc6ncc(F)c(-c7cccc(N8CCOCC8)c7)n6)CC5)CC4)CC3)c(F)c2)C(=O)N1]

Synthesis of trans-(1r,4R)—N-((1r,4R)-4-(4-(4-((2,6-dioxopiperidin-3-yl)amino)-2-fluorophenyl)piperazin-1-yl)cyclohexyl)-4-((5-fluoro-4-(3-morpholinophenyl)pyrimidin-2-yl)amino)cyclohexane-1-carboxamide (Compound 661). To a solution of trans-4-((5-fluoro-4-(3-morpholinophenyl)pyrimidin-2-yl)amino)cyclohexane-1-carboxylic acid (100 mg, 250 μmol, 0.420 eq) in DMF (4 mL) were added HATU (79.2 mg, 208 μmol, 0.350 eq), DIEA (385 mg, 2.98 mmol, 518 μL, 5.00 eq) and 3-((4-(4-(4-aminocyclohexyl)piperazin-1-yl)-3-fluorophenyl)amino)piperidine-2,6-dione (308 mg, 595 μmol, 1.00 eq, TFA) at 25 °C. The mixture was stirred at 25 °C for 1 h. LC-MS showed trans-4-((5-fluoro-4-(3-morpholinophenyl)pyrimidin-2-yl)amino)cyclohexane-1-carboxylic acid and the desired mass was detected. The reaction mixture was concentrated under reduced pressure to give a residue. The residue was purified by prep-HPLC (column: Phenomenex luna C18 100*40 mm*5 um; mobile phase: [H₂O (0.2% FA)-ACN]; gradient: 20%-55% B over 8.0 min) to give the title compound (Compound 661) (74.7 mg, 86.8 μmol, 14.6% yield, 96.6% purity, FA) as a yellow solid. LCMS (ES⁺): m/z 786.5 [M+H]⁺

### Example 676. Synthesis of trans-(1r,4R)—N-((1r,4R)-4-(4-(4-((2,6-dioxopiperidin-3-yl)amino)-2-fluorophenyl)piperidin-1-yl)cyclohexyl)-4-((5-fluoro-4-(3-morpholinophenyl)pyrimidin-2-yl)amino)cyclohexane-1-carboxamide (Compound 657)

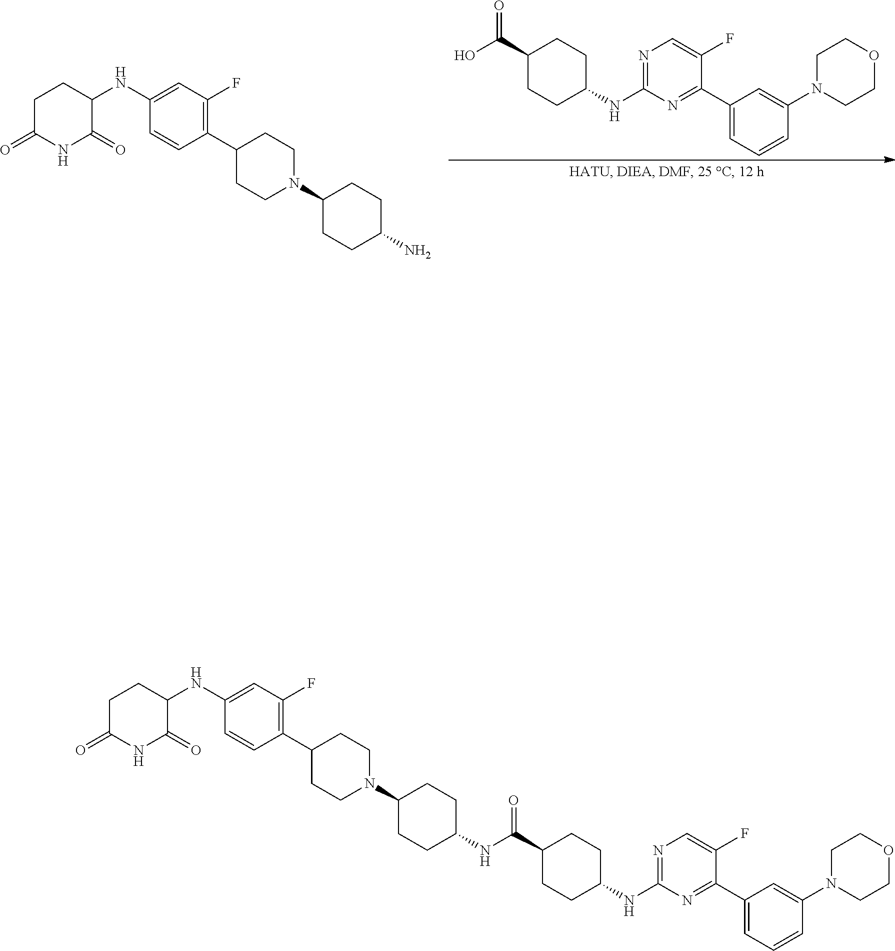 [SMILES: N[C@H]1CC[C@H](N2CCC(c3ccc(NC4CCC(=O)NC4=O)cc3F)CC2)CC1 ; O=C(O)[C@H]1CC[C@H](Nc2ncc(F)c(-c3cccc(N4CCOCC4)c3)n2)CC1 ; O=C1CCC(Nc2ccc(C3CCN([C@H]4CC[C@H](NC(=O)[C@H]5CC[C@H](Nc6ncc(F)c(-c7cccc(N8CCOCC8)c7)n6)CC5)CC4)CC3)c(F)c2)C(=O)N1]

Synthesis of trans-(1r,4R)—N-((1r,4R)-4-(4-(4-((2,6-dioxopiperidin-3-yl)amino)-2-fluorophenyl)piperidin-1-yl)cyclohexyl)-4-((5-fluoro-4-(3-morpholinophenyl)pyrimidin-2-yl)amino)cyclohexane-1-carboxamide (Compound 657). To a solution of trans-4-((5-fluoro-4-(3-morpholinophenyl)pyrimidin-2-yl)amino)cyclohexane-1-carboxylic acid (86.1 mg, 215 μmol, 0.600 eq) in DMF (4 mL) were added DIEA (232 mg, 1.79 mmol, 313 μL, 5.00 eq) and HATU (136 mg, 358 μmol, 1.00 eq) at 25 °C. Then to the mixture was added trans-3-((4-(1-(4-aminocyclohexyl)piperidin-4-yl)-3-fluorophenyl)amino)piperidine-2,6-dione (185 mg, 358 μmol, 1.00 eq, TFA) at 25 °C. The mixture was stirred at 25 °C for 12 h. LCMS showed trans-3-((4-(1-(4-aminocyclohexyl)piperidin-4-yl)-3-fluorophenyl)amino)piperidine-2,6-dione was consumed and the desired mass was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 25%-65% B over 8.0 min) to give the title compound (Compound 657) (31.9 mg, 37.0 μmol, 10.3% yield, 91.1% purity) as a pale yellow solid. LCMS (ES⁺): m/z 785.5 [M+H]⁺

### Example 677. Synthesis of trans-1′-(4-((2,6-dioxopiperidin-3-yl)amino)-2-fluorophenyl)-N-(4-((5-fluoro-4-(3-morpholinophenyl)pyrimidin-2-yl)amino)cyclohexyl)-[1,4′-bipiperidine]-4-carboxamide (Compound 645)

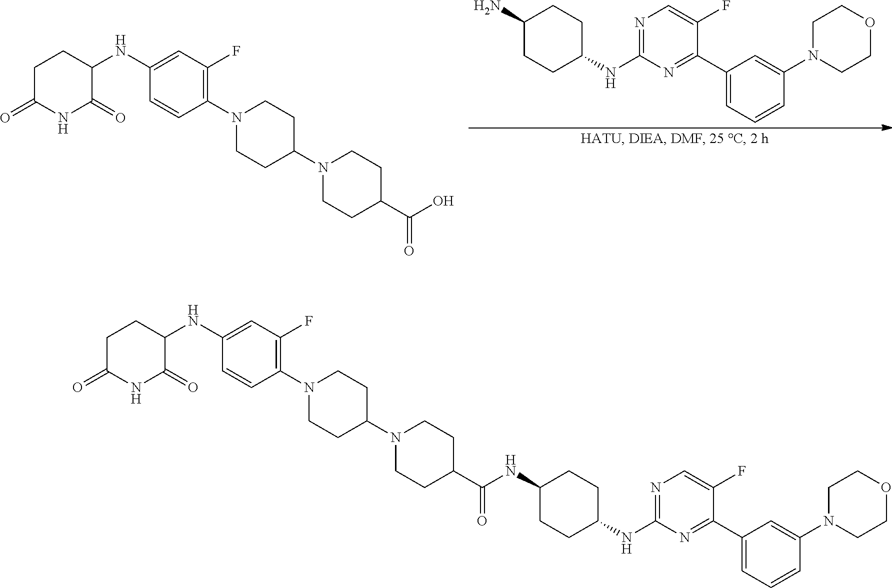 [SMILES: O=C1CCC(Nc2ccc(N3CCC(N4CCC(C(=O)O)CC4)CC3)c(F)c2)C(=O)N1 ; N[C@H]1CC[C@H](Nc2ncc(F)c(-c3cccc(N4CCOCC4)c3)n2)CC1 ; O=C1CCC(Nc2ccc(N3CCC(N4CCC(C(=O)N[C@H]5CC[C@H](Nc6ncc(F)c(-c7cccc(N8CCOCC8)c7)n6)CC5)CC4)CC3)c(F)c2)C(=O)N1]

Synthesis of trans-1′-(4-((2,6-dioxopiperidin-3-yl)amino)-2-fluorophenyl)-N-(4-((5-fluoro-4-(3-morpholinophenyl)pyrimidin-2-yl)amino)cyclohexyl)-[1,4′-bipiperidine]-4-carboxamide (Compound 645). To a solution of 1′-(4-((2,6-dioxopiperidin-3-yl)amino)-2-fluorophenyl)-[1,4′-bipiperidine]-4-carboxylic acid (200 mg, 462 μmol, 1.00 eq) in DMF (5 mL) were added HATU (176 mg, 462 μmol, 1.00 eq) and DIEA (120 mg, 925 μmol, 161 μL, 2.00 eq) at 25 °C, and then trans-N1-(5-fluoro-4-(3-morpholinophenyl)pyrimidin-2-yl)cyclohexane-1,4-diamine (112 mg, 231 μmol, 0.500 eq, TFA) was added at 25 °C. The resulting mixture was stirred at 25 °C for 2 h. LCMS showed that 1′-(4-((2,6-dioxopiperidin-3-yl)amino)-2-fluorophenyl)-[1,4′-bipiperidine]-4-carboxylic acid was consumed completely and the desired mass was detected. The mixture was concentrated under reduced pressure to give a residue. The residue was purified by prep-HPLC (column: Waters Xbridge BEH C18 100*30 mm*10 um; mobile phase: [H₂O (10 mM NH₃HCO₃)-ACN]; gradient: 20%-50% B over 8.0 min) to give the title compound (Compound 645) (56.5 mg, 68.8 μmol, 14.9% yield, 95.8% purity) as a white solid. LCMS (ESI+): m/s 786.4 [M+H]⁺

### Example 678. Synthesis of 1′-(4-((2,6-dioxopiperidin-3-yl)amino)-2-fluorophenyl)-3′-fluoro-N-((1r,4r)-4-((5-fluoro-4-(3-morpholinophenyl)pyrimidin-2-yl)amino)cyclohexyl)-[1,4′-bipiperidine]-4-carboxamide (Compound 655)

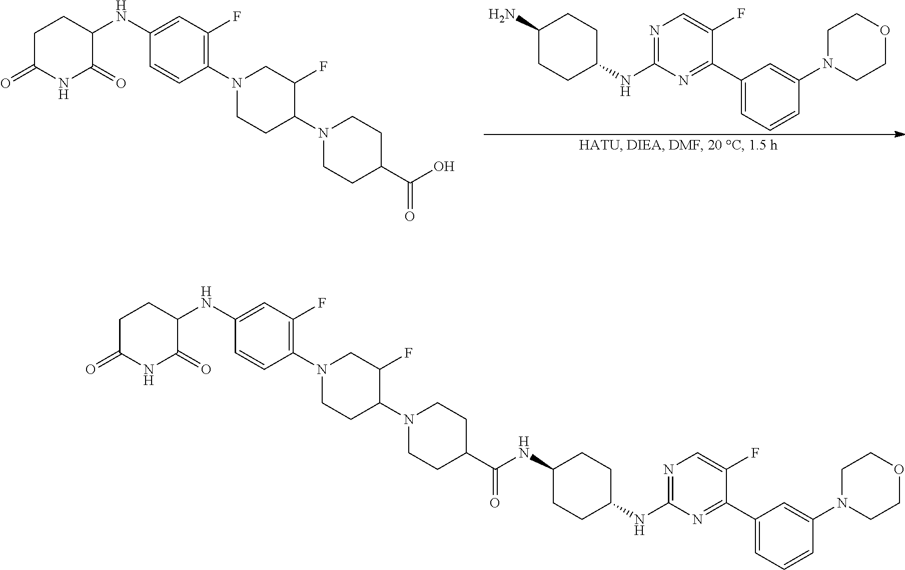 [SMILES: O=C1CCC(Nc2ccc(N3CCC(N4CCC(C(=O)O)CC4)C(F)C3)c(F)c2)C(=O)N1 ; N[C@H]1CC[C@H](Nc2ncc(F)c(-c3cccc(N4CCOCC4)c3)n2)CC1 ; O=C1CCC(Nc2ccc(N3CCC(N4CCC(C(=O)N[C@H]5CC[C@H](Nc6ncc(F)c(-c7cccc(N8CCOCC8)c7)n6)CC5)CC4)C(F)C3)c(F)c2)C(=O)N1]

Synthesis of 1′-(4-((2,6-dioxopiperidin-3-yl)amino)-2-fluorophenyl)-3′-fluoro-N-((1r,4r)-4-((5-fluoro-4-(3-morpholinophenyl)pyrimidin-2-yl)amino)cyclohexyl)-[1,4′-bipiperidine]-4-carboxamide (Compound 655). To a solution of 1′-(4-((2,6-dioxopiperidin-3-yl)amino)-2-fluorophenyl)-3′-fluoro-[1,4′-bipiperidine]-4-carboxylic acid (133 mg, 295 μmol, 1.00 eq) in DMF (4 mL) were added HATU (39.3 mg, 103 μmol, 0.350 eq) and DIEA (194 mg, 1.51 mmol, 262 μL, 5.00 eq) at 20 °C, then trans-N1-(5-fluoro-4-(3-morpholinophenyl)pyrimidin-2-yl)cyclohexane-1,4-diamine (86.0 mg, 177 μmol, 0.600 eq, TFA) was added to the mixture, the mixture was stirred at 20 °C for 1.5 h. LCMS showed 1′-(4-((2,6-dioxopiperidin-3-yl)amino)-2-fluorophenyl)-3′-fluoro-[1,4′-bipiperidine]-4-carboxylic acid was consumed completely and the desired target MS was detected. The mixture was concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 35%-65% B over 8.0 min) to give the title compound (Compound 655) (49.0 mg, 56.7 μmol, 19.2% yield, 93.0% purity) as a pale yellow solid. LCMS (ES+): m/z 804.4 [M+H]⁺

### Example 679. Synthesis of trans-(1r,4R)-4-(4-(4-((2,6-dioxopiperidin-3-yl)amino)-2-fluorophenyl)piperazin-1-yl)-N-((1r,4R)-4-((5-fluoro-4-(3-morpholinophenyl)pyrimidin-2-yl)amino)cyclohexyl)cyclohexane-1-carboxamide (Compound 605)

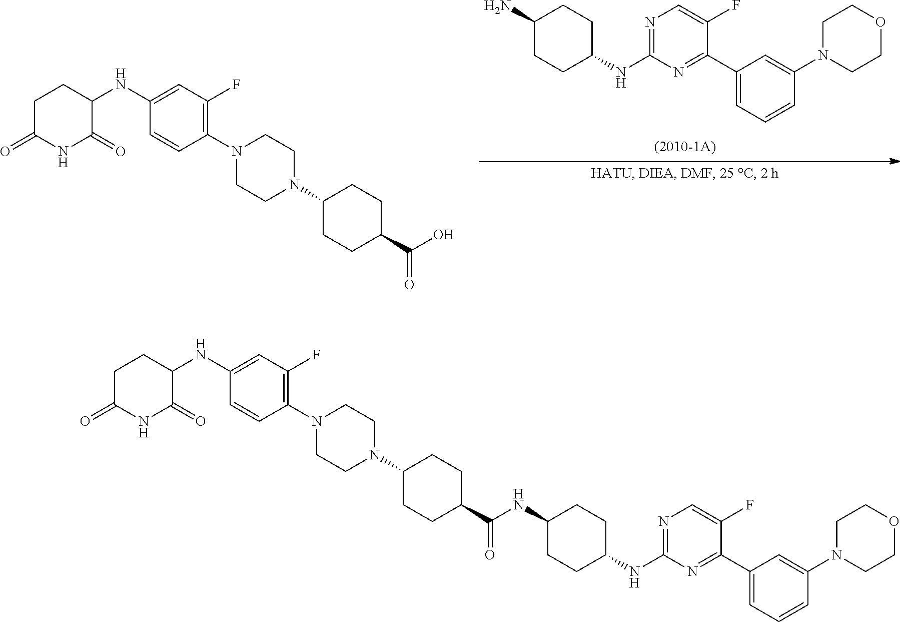 [SMILES: O=C1CCC(Nc2ccc(N3CCN([C@H]4CC[C@H](C(=O)O)CC4)CC3)c(F)c2)C(=O)N1 ; N[C@H]1CC[C@H](Nc2ncc(F)c(-c3cccc(N4CCOCC4)c3)n2)CC1 ; O=C1CCC(Nc2ccc(N3CCN([C@H]4CC[C@H](C(=O)N[C@H]5CC[C@H](Nc6ncc(F)c(-c7cccc(N8CCOCC8)c7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperazin-1-yl)-N-((1r,4R)-4-((5-fluoro-4-(3-morpholinophenyl)pyrimidin-2-yl)amino)cyclohexyl)cyclohexane-1-carboxamide (Compound 605). To a solution of trans-4-(4-(4-((2,6-dioxopiperidin-3-yl)amino)-2-fluorophenyl)piperazin-1-yl)cyclohexane-1-carboxylic acid (200 mg, 462 μmol, 1.00 eq) in DMF (7 mL) were added DIEA (299 mg, 2.31 mmol, 403 μL, 5.00 eq), HATU (87.0 mg, 231 μmol, 0.500 eq) and trans-N1-(5-fluoro-4-(3-morpholinophenyl)pyrimidin-2-yl)cyclohexane-1,4-diamine (67.4 mg, 139 μmol, 0.300 eq, TFA) at 25 °C. The mixture was stirred at 25 °C for 2 h. LCMS showed trans-4-(4-(4-((2,6-dioxopiperidin-3-yl)amino)-2-fluorophenyl)piperazin-1-yl)cyclohexane-1-carboxylic acid was consumed and the desired mass was detected. The reaction mixture was concentrated under reduced pressure to give a residue. The residue were purified by prep-HPLC (column: Phenomenex luna C18 100*40 mm*3 um; mobile phase: [H₂O (0.2% FA)-ACN]; gradient: 5%-40% B over 8.0 min) to give the title compound (Compound 605) (72.3 mg, 81.9 μmol, 17.7% yield, 94.3% purity, FA) as a yellow solid. LCMS (ES⁺): m/z 786.5 [M+H]⁺.

### Example 680. Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(3-morpholinophenyl)pyrimidin-2-yl)amino)cyclohexyl)cyclohexane-1-carboxamide (Compound 659)

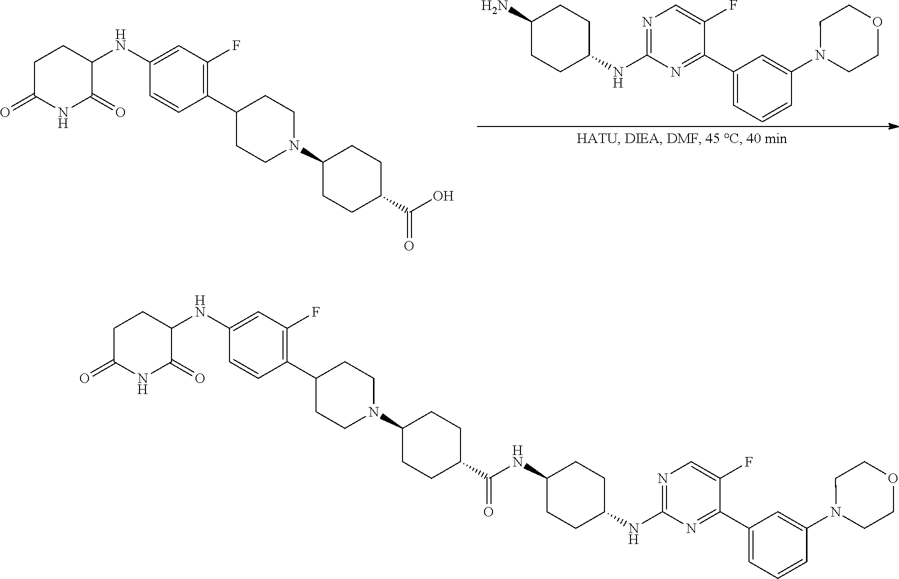 [SMILES: O=C1CCC(Nc2ccc(C3CCN([C@H]4CC[C@H](C(=O)O)CC4)CC3)c(F)c2)C(=O)N1 ; N[C@H]1CC[C@H](Nc2ncc(F)c(-c3cccc(N4CCOCC4)c3)n2)CC1 ; O=C1CCC(Nc2ccc(C3CCN([C@H]4CC[C@H](C(=O)N[C@H]5CC[C@H](Nc6ncc(F)c(-c7cccc(N8CCOCC8)c7)n6)CC5)CC4)CC3)c(F)c2)C(=O)N1]

Synthesis of trans-(1r,4R)-4-(4-(4-((2,6-dioxopiperidin-3-yl)amino)-2-fluorophenyl)piperidin-1-yl)-N-((1r,4R)-4-((5-fluoro-4-(3-morpholinophenyl)pyrimidin-2-yl)amino)cyclohexyl)cyclohexane-1-carboxamide (Compound 659). To a solution of trans-4-(4-(4-((2,6-dioxopiperidin-3-yl)amino)-2-fluorophenyl)piperidin-1-yl)cyclohexane-1-carboxylic acid (130 mg, 301 μmol, 1.00 eq) in DMF (4 mL) were added DIEA (195 mg, 1.51 mmol, 1263 μL, 5.00 eq) and HATU (115 mg, 301 μmol, 1.00 eq) at 25 °C, the mixture was stirred at 45 °C for 10 min. Then trans-N-(5-fluoro-4-(3-morpholinophenyl)pyrimidin-2-yl)cyclohexane-1,4-diamine (132 mg, 271 μmol, 0.900 eq, TFA) was added at 25 °C, the mixture was stirred at 45 °C for 30 min. LCMS showed that trans-4-(4-(4-((2,6-dioxopiperidin-3-yl)amino)-2-fluorophenyl)piperidin-1-yl)cyclohexane-1-carboxylic acid was consumed and the desired mass was detected. The mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 25%-55% B over 8.0 min) to give the title compound (Compound 659) (79.8 mg, 96.0 μmol, 31.9% yield, 94.5% purity) as a white solid. LCMS (ES⁺): m/z 785.5 [M+H]⁺.

TABLE 3
Spectrometric data for compounds of Table 1

| Cmpd | MS | MS | |
|---|---|---|---|
| No. | Calc. | Found | ¹H NMR Data (400 MHz, DMSO-d₆) |
| 1 | 406 | 405 | δ 8.41 (s, 1H), 7.98 (s, 1H), 7.79 (d, J = 7.8 Hz, 1H), 7.76-7.66 |
| | | | (m, 3H), 7.63-7.56 (m, 1H), 7.49 (t, J = 7.6 Hz, 2H), 7.43- |
| | | | 7.37 (m, 1H), 3.89 (br s, 1H), 2.19 (dt, J = 8.2, 4.1 Hz, 1H), |
| | | | 1.94-1.68 (m, 4H), 1.67-1.44 (m, 4H) |
| 2 | 475 | 476 | δ 8.46 (br s, 1H), 8.00 (br s, 1H), 7.81 (d, J = 7.8 Hz, 1H), 7.71 |
| | | | (br d, J = 7.5 Hz, 3H), 7.61 (br t, J = 7.7 Hz, 2H), 7.50 (s, 2H), |
| | | | 7.41 (s, 1H), 4.35-4.23 (m, 1H), 3.98 (br s, 2H), 3.15 (br d, J = |
| | | | 11.0 Hz, 3H), 2.91-2.83 (m, 1H), 2.76 (br t, J = 11.0 Hz, 3H), |
| | | | 2.00-1.87 (m, 2H), 1.65 (br s, 4H), 1.52-1.26 (m, 2H) |
| 3 | 489 | 490 | δ 8.46 (s, 1H), 8.00 (br s, 1H), 7.81 (td, J = 7.7, 1.5 Hz, 1H), |
| | | | 7.77-7.69 (m, 3H), 7.65-7.55 (m, 2H), 7.51 (t, J = 7.6 Hz, |
| | | | 2H), 7.44-7.38 (m, 1H), 4.36-4.24 (m, 1H), 4.05-3.84 (m, |
| | | | 2H), 3.21-3.09 (m, 1H), 2.76 (br d, J = 9.7 Hz, 3H), 2.54 (br s, |
| | | | 1H), 2.14 (s, 3H), 1.98-1.84 (m, 4H), 1.55 (br s, 4H), 1.46- |
| | | | 1.27 (m, 2H) |
| 4 | 517 | 518 | δ 8.46 (s, 1H), 8.00 (br s, 1H), 7.81 (d, J = 7.9 Hz, 1H), 7.76- |
| | | | 7.69 (m, 3H), 7.61 (br t, J = 7.7 Hz, 2H), 7.50 (t, J = 7.6 Hz, |
| | | | 2H), 7.44-7.37 (m, 1H), 4.41-4.22 (m, 2H), 3.99 (br dd, J = |
| | | | 3.1, 7.4 Hz, 2H), 3.80 (br d, J = 12.9 Hz, 1H), 3.25-3.04 (m, |
| | | | 2H), 2.97-2.87 (m, 1H), 2.76 (br t, J = 11.9 Hz, 1H), 2.61 (br t, |
| | | | J = 12.4 Hz, 1H), 1.99 (s, 3H), 1.97-1.85 (m, 2H), 1.61 (br s, |
| | | | 2H), 1.55-1.43 (m, 2H), 1.34 (br d, J = 7.1 Hz, 2H) |
| 5 | 489 | 490 | δ 9.15-8.56 (m, 1H), 8.48 (br s, 1H), 8.02 (br s, 1H), 7.83 (br |
| | | | d, J = 7.7 Hz, 1H), 7.73 (br d, J = 7.6 Hz, 3H), 7.63 (br t, J = |
| | | | 7.6 Hz, 2H), 7.53 (t, J = 7.6 Hz, 2H), 7.47-7.41 (m, 1H), 4.40- |
| | | | 4.24 (m, 1H), 4.06-3.96 (m, 1H), 3.94-3.84 (m, 1H), 3.25 (br |
| | | | d, J = 12.0 Hz, 2H), 3.15 (br t, J = 11.9 Hz, 1H), 2.87 (br t, J = |
| | | | 12.2 Hz, 2H), 2.78 (br t, J = 11.6 Hz, 1H), 2.32 (br d, J = 6.6 |
| | | | Hz, 2H), 2.04-1.88 (m, 3H), 1.84 (br d, J = 13.3 Hz, 2H), 1.53- |
| | | | 1.25 (m, 4H) |
| 6 | 531 | 532 | δ 8.54-8.42 (m, 1H), 8.03 (br dd, J = 4.5, 1.6 Hz, 1H), 7.87- |
| | | | 7.81 (m, 1H), 7.78-7.70 (m, 3H), 7.68-7.58 (m, 2H), 7.57 (br |
| | | | s, 2H), 7.48-7.39 (m, 1H), 4.33 (br d, J = 13.9 Hz, 2H), 4.05- |
| | | | 3.89 (m, 2H), 3.84-3.74 (m, 1H), 3.20-3.10 (m, 1H), 3.06- |
| | | | 2.96 (m, 1H), 2.81-2.72 (m, 1H), 2.33-2.23 (m, 2H), 2.03- |
| | | | 1.85 (m, 7H), 1.75-1.62 (m, 2H), 1.49-1.31 (m, 2H), 1.20 |
| 7 | 503 | 504 | δ 8.45 (s, 1H), 8.00 (br s, 1H), 7.80 (s, 1H), 7.71 (br d, J = 7.5 |
| | | | Hz, 3H), 7.61 (br t, J = 7.7 Hz, 2H), 7.53-7.48 (m, 2H), 7.41 |
| | | | (s, 1H), 4.29 (br d, J = 8.8 Hz, 1H), 4.02-3.93 (m, 1H), 3.90- |
| | | | 3.80 (m, 1H), 3.19-3.08 (m, 3H), 2.70 (br d, J = 11.0 Hz, 3H), |
| | | | 2.36-2.30 (m, 2H), 1.91 (br s, 2H), 1.75 (br d, J = 13.0 Hz, |
| | | | 2H), 1.43 (br d, J = 5.9 Hz, 4H), 1.34 (br s, 1H), 1.25-1.11 (m, |
| | | | 2H) |
| 8 | 545 | 546 | δ 8.45 (s, 1H), 8.03-7.97 (m, 1H), 7.83-7.79 (m, 1H), 7.76- |
| | | | 7.69 (m, 3H), 7.65-7.57 (m, 2H), 7.51 (s, 2H), 7.43-7.38 (m, |
| | | | 1H), 4.40-4.25 (m, 2H), 4.03-3.93 (m, 1H), 3.91-3.82 (m, |
| | | | 1H), 3.81-3.73 (m, 1H), 3.17-3.07 (m, 1H), 3.00-2.90 (m, |
| | | | 1H), 2.80-2.68 (m, 1H), 2.47-2.40 (m, 1H), 2.37-2.30 (m, |
| | | | 2H), 1.97 (s, 3H), 1.95-1.82 (m, 2H), 1.73-1.63 (m, 2H), 1.49- |
| | | | 1.39 (m, 4H), 1.37-1.28 (m, 1H), 1.11-1.00 (m, 1H), 0.97- |
| | | | 0.86 (m, 1H) |
| 9 | 503 | 504 | δ 8.39 (s, 1H), 8.32 (s, 1H), 7.93 (br s, 1H), 7.78 (br d, J = 7.6 |
| | | | Hz, 1H), 7.73-7.63 (m, 3H), 7.62-7.56 (m, 1H), 7.48 (t, J = |
| | | | 7.6 Hz, 2H), 7.42-7.35 (m, 1H), 4.32-4.19 (m, 1H), 3.97- |
| | | | 3.91 (m, 1H), 3.84-3.79 (m, 1H), 3.21 (br d, J = 12.1 Hz, 2H), |
| | | | 3.10 (br t, J = 12.1 Hz, 1H), 2.79-2.70 (m, 3H), 2.61 (s, 3H), |
| | | | 2.28 (br d, J = 6.5 Hz, 2H), 1.98-1.84 (m, 3H), 1.80 (br d, J = |
| | | | 14.5 Hz, 2H), 1.43-1.27 (m, 4H) |
| 10 | 517 | 518 | δ 8.48-8.43 (m, 1H), 8.00 (br s, 1H), 7.80 (s, 1H), 7.76-7.69 |
| | | | (m, 3H), 7.61 (s, 2H), 7.51 (t, J = 7.6 Hz, 2H), 7.41 (s, 1H), |
| | | | 4.34-4.21 (m, 1H), 4.04-3.92 (m, 1H), 3.91-3.81 (m, 1H), |
| | | | 3.31-3.27 (m, 2H), 3.13 (br s, 2H), 2.83 (br s, 3H), 2.35 (br s, |
| | | | 2H), 2.07-1.71 (m, 5H), 1.45 (br d, J = 6.0 Hz, 8H) |
| 11 | 543 | 544 | δ 8.50-8.38 (m, 2H), 8.00 (br s, 1H), 7.81 (dd, J = 7.7, 1.2 Hz, |
| | | | 1H), 7.73-7.69 (m, 2H), 7.64-7.56 (m, 2H), 7.51 (t, J = 7.6 |
| | | | Hz, 2H), 7.44-7.39 (m, 1H), 4.36-4.22 (m, 1H), 4.05-3.87 |
| | | | (m, 2H), 3.21-3.07 (m, 1H), 2.91 (br d, J = 4.4 Hz, 4H), 2.73 |
| | | | (br t, J = 11.4 Hz, 1H), 2.61-2.54 (m, 1H), 2.01-1.83 (m, 2H), |
| | | | 1.74-1.62 (m, 2H), 1.57 (br s, 2H), 1.51-1.37 (m, 7H), 1.35- |
| | | | 1.29 (m, 1H), 1.18 (br t, J = 11.3 Hz, 2H), |
| 12 | 557 | 558 | δ 8.45 (br s, 1H), 8.29 (s, 1H), 8.00 (br s, 1H), 7.81 (m, 1H), |
| | | | 7.76-7.71 (m, 2H), 7.70 (s, 1H), 7.61 (br t, J = 7.8 Hz, 2H), |
| | | | 7.53-7.48 (m, 2H), 7.44-7.38 (m, 1H), 4.33-4.26 (m, 1H), |
| | | | 3.99-3.88 (m, 3H), 3.14 (br t, J = 11.5 Hz, 2H), 2.98 (br d, J = |
| | | | 5.8 Hz, 1H), 2.72 (br t, J = 11.2 Hz, 1H), 2.60-2.53 (m, 1H), |
| | | | 2.39 (br d, J = 3.9 Hz, 3H), 2.24 (s, 3H), 1.97-1.86 (m, 2H), |
| | | | 1.64 (br d, J = 12.1 Hz, 2H), 1.50 (br s, 2H), 1.43 (br s, 3 H), |
| | | | 1.35-1.29 (m, 3H), 1.18-1.08 (m, 2H) |
| 13 | 585 | 586 | δ 8.45 (br s, 1H), 8.15 (s, 1H), 8.00 (br s, 1H), 7.81 (br d, J = |
| | | | 8.1 Hz, 1H), 7.72 (br d, J = 1.2 Hz, 2H), 7.70 (s, 1H), 7.61 (br t, |
| | | | J = 7.8 Hz, 2H), 7.51 (t, J = 7.6 Hz, 2H), 7.43-7.38 (m, 1H), |
| | | | 4.34-4.26 (m, 1H), 4.02-3.88 (m, 2H), 3.43-3.37 (m, 4H), |
| | | | 3.14 (br t, J = 12.5 Hz, 1H), 2.73 (br t, J = 11.8 Hz, 1H), 2.62- |
| | | | 2.54 (m, 1H), 1.97 (d, J = 2.5 Hz, 3H), 1.95-1.86 (m, 2H), |
| | | | 1.69 (br d, J = 11.1 Hz, 2H), 1.46 (br d, J = 2.8 Hz, 4H), 1.41- |
| | | | 1.36 (m, 2H), 1.31-1.25 (m, 2H), 1.23-1.12 (m, 4H) |
| 14 | 392 | 393 | δ 8.50 (s, 1H), 8.03 (br s, 1H), 7.82 (br d, J = 7.9 Hz, 1H), 7.78 |
| | | | (br d, J = 6.9 Hz, 2H), 7.72 (d, J = 7.3 Hz, 2H), 7.65-7.59 (m, |
| | | | 1H), 7.53-7.48 (m, 2H), 7.44-7.38 (m, 1H), 4.28-4.15 (m, |
| | | | 1H), 3.55 (br dd, J = 11.2, 4.1 Hz, 1H), 3.21 (s, 1H), 2.80 (s, |
| | | | 3H), 2.41-2.29 (m, 2H), 2.03-1.88 (m, 2H) |
| 15 | 378 | 379 | δ 8.47 (s, 1H), 8.01 (s, 1H), 7.81 (d, J = 7.8 Hz, 1H), 7.75 (br d, |
| | | | J = 7.5 Hz, 1H), 7.71 (d, J = 7.4 Hz, 2H), 7.66 (br d, J = 7.1 Hz, |
| | | | 1H), 7.63-7.58 (m, 1H), 7.56-7.46 (m, 3H), 7.43-7.37 (m, |
| | | | 1H), 4.22-4.04 (m, 1H), 3.25-3.12 (m, 2H), 2.55 (br d, J = |
| | | | 5.9 Hz, 2H), 2.24 (dd, J = 17.2, 9.3 Hz, 1H), 2.08-1.99 (m, |
| | | | 1H), 1.67 (br d, J = 7.1 Hz, 1H) |
| 16 | 392 | 393 | δ 8.46 (s, 1H), 8.14 (s, 1H), 8.00 (s, 1H), 7.83-7.78 (m, 1H), |
| | | | 7.74 (br d, J = 7.6 Hz, 1H), 7.72-7.66 (m, 3H), 7.64-7.58 (m, |
| | | | 1H), 7.52-7.47 (m, 2H), 7.44-7.37 (m, 1H), 4.21-4.09 (m, |
| | | | 1H), 3.31-3.20 (m, 2H), 2.80 (s, 3H), 2.60 (br dd, J = 17.0, 4.8 |
| | | | Hz, 1H), 2.29 (dd, J = 17.0, 9.3 Hz, 1H), 2.13-2.06 (m, 1H), |
| | | | 1.85-1.72 (m, 1H) |
| 17 | 378 | 379 | δ 8.51-8.42 (m, 1H), 8.00 (br s, 1H), 7.80 (br d, J = 7.8 Hz, |
| | | | 1H), 7.74 (br d, J = 7.4 Hz, 1H), 7.70 (br d, J = 7.1 Hz, 3H), |
| | | | 7.63-7.57 (m, 1H), 7.49 (t, J = 7.6 Hz, 2H), 7.45-7.36 (m, |
| | | | 2H), 4.14-4.04 (m, 1H), 3.36-3.25 (m, 1H), 3.11 (br dd, J = |
| | | | 10.9, 8.6 Hz, 1H), 2.40-2.17 (m, 2H), 2.04-1.93 (m, 1H), |
| | | | 1.92-1.76 (m, 1H) |
| 18 | 406 | 407 | δ 8.47 (br d, J = 14.5 Hz, 1H), 8.01 (s, 1H), 7.81 (br d, J = 7.8 |
| | | | Hz, 1H), 7.77-7.69 (m, 3H), 7.64-7.58 (m, 1H), 7.50 (t, J = |
| | | | 7.6 Hz, 3H), 7.44-7.39 (m, 1H), 4.09-3.96 (m, 1H), 3.81- |
| | | | 3.62 (m, 2H), 3.09-2.91 (m, 1H), 2.68 (m, 1H), 2.05-1.95 (m, |
| | | | 3H), 1.82-1.72 (m, 2H), 1.59-1.30 (m, 2H) |
| 19 | 406 | 407 | δ 8.49 (br s, 1H), 8.05 (br s, 1H), 7.86 (m, 1H), 7.79-7.74 (m, |
| | | | 3H), 7.76-7.70 (m, 1H), 7.59-7.52 (m, 3H), 7.49-7.44 (m, |
| | | | 1H), 7.25 (br s, 1H), 6.74 (br s, 1H), 3.73 (m, 1H), 2.12-2.01 |
| | | | (m, 3H), 1.85 (br d, J = 11.6 Hz, 2H), 1.51-1.43 (m, 2H), 1.39- |
| | | | 1.30 (m, 2H) |
| 20 | 399 | 400 | δ 7.80 (br d, J = 14.4 Hz, 1H), 7.36-7.30 (m, 4H), 7.28-7.21 |
| | | | (m, 1H), 6.83-6.61 (m, 1H), 4.45-4.06 (m, 1H), 3.99-3.75 |
| | | | (m, 3H), 3.72-3.50 (m, 3H), 3.43-3.38 (m, 1H), 3.03-2.88 |
| | | | (m, 1H), 2.85-2.64 (m, 1H), 2.33-2.22 (m, 1H), 2.07-1.79 |
| | | | (m, 5H), 1.76-1.62 (m, 1H), 1.59-1.21 (m, 2H) |
| 21 | 406 | 405 | δ 8.42-8.28 (m, 1H), 8.10-7.95 (m, 1H), 7.86-7.74 (m, 1H), |
| | | | 7.71 (br d, J = 7.5 Hz, 1H), 7.67-7.60 (m, 2H), 7.59-7.52 (m, |
| | | | 1H), 7.47 (t, J = 7.5 Hz, 2H), 7.42-7.35 (m, 1H), 5.44-5.02 |
| | | | (m, 1H), 4.31- 3.75 (m, 3H), 3.55-3.32 (m, 1H), 3.13 (br d, J = |
| | | | 7.5 Hz, 1H), 2.20-1.96 (m, 4H), 1.81-1.73 (m, 1H), 1.64 (br |
| | | | d, J = 13.6 Hz, 2H) |
| 22 | 406 | 407 | δ 8.33 (s, 1H), 8.02 (s, 1H), 7.78 (br d, J = 7.4 Hz, 1H), 7.71 (d, |
| | | | J = 7.9 Hz, 1H), 7.64 (d, J = 7.4 Hz, 2H), 7.59-7.53 (m, 1H), |
| | | | 7.47 (t, J = 7.6 Hz, 2H), 7.41-7.35 (m, 1H), 5.42-5.21 (m, |
| | | | 2H), 3.99-3.79 (m, 1H), 2.44-2.26 (m, 2H), 2.14 (br d, J = |
| | | | 12.4 Hz, 1H), 1.94 (br dd, J = 6.7, 2.6 Hz, 2H), 1.47 (dt, J = 9.0, |
| | | | 4.5 Hz, 2H), 1.40 (d, J = 11.8 Hz, 1H), 1.28-1.21 (m, 2H) |
| 23 | 406 | 405 | δ 8.45 (s, 1H), 7.99 (br s, 1H), 7.80 (dd, J = 7.8, 1.4 Hz, 1H), |
| | | | 7.72-7.69 (m, 2H), 7.60 (br t, J = 7.7 Hz, 2H), 7.50 (t, J = 7.6 |
| | | | Hz, 2H), 7.43-7.36 (m, 1H), 4.26 (br d, J = 11.9 Hz, 1H), 4.03- |
| | | | 3.91 (m, 1H), 3.80 (br d, J = 13.4 Hz, 1H), 3.20-3.06 (m, |
| | | | 1H), 2.79-2.65 (m, 1H), 1.99 (s, 3H), 1.90 (br dd, J = 19.9, |
| | | | 13.6 Hz, 2H), 1.54-1.23 (m, 2H) |
| 24 | 337 | 338 | δ 8.16 (s, 1H), 7.88 (s, 1H), 6.98-6.83 (m, 1H), 4.28-4.21 (m, |
| | | | 1H), 3.84-3.74 (m, 2H), 3.49 (br s, 4H), 3.13-3.06 (m, 1H), |
| | | | 2.72-2.66 (m, 1H), 1.99 (s, 3H), 1.91-1.79 (m, 2H), 1.60 (br |
| | | | s, 6H), 1.45-1.36 (m, 1H), 1.32-1.23 (m, 1H) |
| 25 | 348 | 349 | δ 8.40 (s, 1H), 7.82 (br s, 2H), 7.42 (br d, J = 6.2 Hz, 1H), 7.35 |
| | | | (t, J = 8.9 Hz, 2H), 7.16 (br s, 1H), 6.70 (br s, 1H), 3.93-3.85 |
| | | | (m, 1H), 2.19 (dq, J = 8.1, 4.1 Hz, 1H), 1.92-1.82 (m, 2H), |
| | | | 1.74 (br s, 2H), 1.60 (br dd, J = 9.5, 3.8 Hz, 2H), 1.55-1.46 (m, |
| | | | 2H) |
| 26 | 348 | 349 | δ = 8.44 (br d, J = 15.7 Hz, 1H), 7.83 (br s, 1H), 7.68-7.48 (m, |
| | | | 1H), 7.40-7.25 (m, 2H), 4.08-3.84 (m, 1H), 3.76-3.59 (m, |
| | | | 2H), 3.09-2.92 (m, 1H), 2.78-2.59 (m, 1H), 1.98-1.83 (m, |
| | | | 3H), 1.80-1.67 (m, 1H), 1.64-1.43 (m, 2H), 1.41-1.23 (m, |
| | | | 1H) |
| 27 | 348 | 349 | δ = 8.42 (s, 1H), 7.80 (br d, J = 1.6 Hz, 1H), 7.59 (br s, 1H), |
| | | | 7.34 (br t, J = 8.9 Hz, 2H), 4.30-4.20 (m, 1H), 4.00-3.89 (m, |
| | | | 1H), 3.79 (br d, J = 13.0 Hz, 1H), 3.18-3.09 (m, 1H), 2.72 (br |
| | | | t, J = 11.2 Hz, 1H), 1.99-1.80 (m, 4H), 1.48-1.21 (m, 3H) |
| 28 | 348 | 349 | δ = 8.39 (br s, 1H), 7.83-7.76 (m, 2H), 7.38-7.31 (m, 3H), |
| | | | 7.22 (br s, 1H), 6.69 (br s, 1H), 2.46-2.43 (m, 1H), 2.23-2.15 |
| | | | (m, 1H), 1.96-1.86 (m, 2H), 1.79-1.74 (m, 1H), 1.71-1.66 |
| | | | (m, 1H), 1.37-1.27 (m, 2H), 1.21-1.15 (m, 2H) |
| 29 | 400 | 401 | δ = 8.52 (br d, J = 4.3 Hz, 1H), 7.88-7.69 (m, 2H), 7.37 (br t, |
| | | | J = 7.9 Hz, 1H), 7.25 (br t, J = 5.7 Hz, 1H), 6.73-6.56 (m, 1H), |
| | | | 4.22-4.05 (m, 1H), 3.99-3.70 (m, 4H), 3.71-3.49 (m, 3H), |
| | | | 3.03-2.87 (m, 1H), 2.84-2.64 (m, 1H), 2.35-2.19 (m, 1H), |
| | | | 2.18-2.04 (m, 1H), 1.98 (s, 1H), 1.89 (br s, 3H), 1.76-1.62 |
| | | | (m, 1H), 1.60-1.25 (m, 2H) |
| 30 | 399 | 400 | δ 8.36-8.20 (m, 1H), 7.35 (br d, J = 7.3 Hz, 1H), 7.22-7.11 |
| | | | (m, 2H), 6.62-6.57 (m, 1H), 6.55 (d, J = 7.9 Hz, 2H), 4.37- |
| | | | 4.01 (m, 1H), 3.94-3.69 (m, 2H), 3.67-3.55 (m, 2H), 3.53 (br |
| | | | s, 1H), 3.44-3.33 (m, 2H), 2.77-2.57 (m, 1H), 2.35 (br dd, J = |
| | | | 10.8, 5.8 Hz, 1H), 2.27-2.05 (m, 2H), 1.95 (br s, 3H), 1.85- |
| | | | 1.56 (m, 2H), 1.48-1.05 (m, 2H) |
| 31 | 420 | 421 | δ 8.44 (s, 1H), 8.00 (br s, 1H), 7.80 (d, J = 7.9 Hz, 1H), 7.78- |
| | | | 7.67 (m, 4H), 7.64-7.57 (m, 1H), 7.50 (t, J = 7.6 Hz, 2H), 7.41 |
| | | | (q, J = 7.2 Hz, 2H), 3.83-3.61 (m, 2H), 1.81 (s, 3H), 1.77- |
| | | | 1.47 (m, 8H) |
| 32 | 420 | 421 | δ 8.42 (br s, 1H), 8.03-7.97 (m, 1H), 7.80 (br d, J = 7.8 Hz, |
| | | | 1H), 7.77-7.68 (m, 4H), 7.64-7.56 (m, 1H), 7.52-7.38 (m, |
| | | | 4H), 3.71-3.43 (m, 2H), 2.01-1.90 (m, 2H), 1.87-1.78 (m, |
| | | | 2H), 1.77 (s, 3H), 1.40-1.16 (m, 4H) |
| 33 | 420 | 421 | δ 8.34 (s, 1H), 8.03 (s, 1H), 7.79 (br d, J = 7.8 Hz, 1H), 7.72 (br |
| | | | d, J = 8.4 Hz, 1H), 7.65 (d, J = 7.3 Hz, 2H), 7.59-7.55 (m, |
| | | | 1H), 7.47 (t, J = 7.4 Hz, 2H), 7.40-7.36 (m, 1H), 5.60-5.46 |
| | | | (m, 1H), 5.32 (br d, J = 8.1 Hz, 1H), 4.13-3.85 (m, 2H), 2.45- |
| | | | 2.42 (m, 1H), 2.16-2.13 (m, 1H), 2.04-2.02 (m, 2H), 1.97 (s, |
| | | | 3H), 1.20-1.05 (m, 4H) |
| 34 | 420 | 421 | δ 8.33 (s, 1H), 8.02 (t, J = 1.6 Hz, 1H), 7.79-7.72 (m, 2H), |
| | | | 7.68-7.64 (m, 2H), 7.59-7.54 (m, 1H), 7.49-7.44 (m, 2H), |
| | | | 7.39-7.34 (m, 1H), 4.20-4.03 (m, 2H), 1.89 (br s, 3H), 1.86- |
| | | | 1.77 (m, 3H), 1.75-1.64 (m, 4H), 1.56-1.46 (m, 1H) |
| 35 | 413 | 414 | δ 8.15 (s, 1H), 7.93 (br d, J = 14.2 Hz, 1H), 7.37-7.28 (m, 4H), |
| | | | 7.25 (br d, J = 6.2 Hz, 1H), 7.15-6.83 (m, 1H), 4.42-4.24 (m, |
| | | | 2H), 4.10-3.97 (m, 1H), 3.80 (br d, J = 12.8 Hz, 1H), 3.66 (br |
| | | | d, J = 9.8 Hz, 2H), 3.03-2.84 (m, 4H), 2.77-2.65 (m, 1H), |
| | | | 1.94 (br s, 3H), 1.86-1.64 (m, 5H), 1.59-1.26 (m, 2H) |
| 36 | 383 | 384 | δ 8.32 (d, J = 3.9 Hz, 1H), 7.30-7.23 (m, 1H), 7.18 (br d, J = |
| | | | 0.9 Hz, 1H), 7.17-6.99 (m, 3H), 6.70-6.61 (m, 2H), 3.72- |
| | | | 3.60 (m, 1H), 3.23 (br t, J = 6.4 Hz, 4H), 2.23-2.13 (m, 1H), |
| | | | 1.94 (br t, J = 6.5 Hz, 6H), 1.78-1.65 (m, 2H), 1.33-1.13 (m, |
| | | | 4H) |
| 37 | 413 | 414 | δ 8.41 (br d, J = 14.4 Hz, 1H), 7.70-7.42 (m, 1H), 7.35-7.27 |
| | | | (m, 1H), 7.22 (br s, 1H), 7.06 (br d, J = 8.4 Hz, 2H), 4.39-4.21 |
| | | | (m, 1H), 4.10-3.80 (m, 1H), 3.79-3.58 (m, 2H), 3.25-3.09 |
| | | | (m, 5H), 3.08-2.87 (m, 1H), 2.65 (br dd, J = 12.2, 9.8 Hz, 1H), |
| | | | 2.03-1.84 (m, 4H), 1.81-1.68 (m, 1H), 1.62 (br d, J = 3.8 Hz, |
| | | | 5H), 1.55 (br d, J = 4.8 Hz, 1H), 1.41-1.27 (m, 1H) |
| 38 | 368 | 369 | δ 8.42 (d, J = 3.7 Hz, 1H), 7.89-7.82 (m, 1H), 7.78 (br d, J = |
| | | | 7.1 Hz, 1H), 7.51-7.45 (m, 1H), 7.44-7.39 (m, 1H), 7.28- |
| | | | 7.22 (m, 2H), 6.71 (br s, 1H), 3.79-3.69 (m, 1H), 3.66-3.57 |
| | | | (m, 1H), 2.40-2.31 (m, 2H), 2.28-2.19 (m, 1H), 2.17-2.09 |
| | | | (m, 2H), 2.07-1.93 (m, 3H), 1.90-1.78 (m, 2H), 1.74 (br d, J = |
| | | | 12.0 Hz, 1H), 1.41-1.30 (m, 2H), 1.28-1.15 (m, 2H) |
| 39 | 412 | 413 | δ 8.43 (br d, J = 14.8 Hz, 1H), 7.60 (br s, 1H), 7.56 (br d, J = |
| | | | 8.8 Hz, 2H), 7.44-7.37 (m, 2H), 4.11-3.98 (m, 1H), 3.76- |
| | | | 3.63 (m, 2H), 3.11-2.91 (m, 1H), 2.71-2.61 (m, 1H), 2.02- |
| | | | 1.93 (m, 4H), 1.81 (br s, 5H), 1.71 (br d, J = 12.5 Hz, 2H), |
| | | | 1.49-1.36 (m, 6H), 1.31-1.21 (m, 1H) |
| 40 | 407 | 408 | δ 8.45 (d, J = 3.6 Hz, 1H), 8.04 (d, J = 7.1 Hz, 1H), 7.98 (s, |
| | | | 1H), 7.74-7.66 (m, 2H), 7.62-7.52 (m, 2H), 7.30 (br d, J = |
| | | | 7.8 Hz, 1H), 7.21 (br s, 1H), 6.67 (br s, 1H), 6.51 (d, J = 9.0 Hz, |
| | | | 1H), 6.35 (dt, J = 1.2, 6.7 Hz, 1H), 3.78-3.64 (m, 1H), 2.26- |
| | | | 2.14 (m, 1H), 2.03-1.86 (m, 2H), 1.81-1.65 (m, 2H), 1.37- |
| | | | 1.13 (m, 4H) |
| 41 | 245 | 246 | δ = 8.43-8.23 (m, 2H), 8.15 (dd, J = 1.9, 9.6 Hz, 1H), 7.69- |
| | | | 7.49 (m, 2H), 7.48-7.32 (m, 2H), 7.28-7.15 (m, 2H), 6.67 (br |
| | | | d, J = 9.6 Hz, 2H), 3.69 (br dd, J = 7.8, 3.7 Hz, 1H), 2.27-2.13 |
| | | | (m, 1H), 2.03-1.86 (m, 2H), 1.83-1.63 (m, 2H), 1.42-1.08 |
| | | | (m, 4H) |
| 42 | 398 | 399 | δ 8.63 (d, J = 2.5 Hz, 1 H), 8.42 (d, J = 3.1 Hz, 1 H), 7.99-7.91 |
| | | | (m, 2 H), 7.41 (t, J = 8.7 Hz, 2 H), 7.22 (br s, 2 H), 7.07 (s, 1 |
| | | | H), 6.68 (br s, 1 H), 3.83-3.65 (m, 1 H), 2.23 (br t, J = 11.7 |
| | | | Hz, 1 H), 2.07-1.87 (m, 2 H), 1.83-1.62 (m, 2 H), 1.26-1.23 |
| | | | (m, 1 H), 1.42-1.06 (m, 3 H) |
| 43 | 398 | 399 | δ 8.92 (d, J = 1.4 Hz, 1 H), 8.37 (d, J = 2.9 Hz, 1 H), 8.28 (s, 1 |
| | | | H), 8.03-7.95 (m, 2 H), 7.44-7.33 (m, 2 H), 7.22 (br s, 1 H), |
| | | | 7.07 (d, J = 8.1 Hz, 1 H), 6.67 (br s, 1 H), 3.86-3.61 (m, 1 H), |
| | | | 2.28-2.18 (m, 1 H), 2.07-1.86 (m, 2 H), 1.84-1.62 (m, 2 H), |
| | | | 1.45-1.04 (m, 4 H) |
| 91 | 415 | 416 | δ 8.26-8.23 (m, 1H), 7.23 (br s, 1H), 7.12 (br s, 5H), 6.71 (br |
| | | | s, 1H), 3.70 (br s, 1H), 3.65-3.60 (m, 3H), 3.17 (br d, J = 7.9 |
| | | | Hz, 1H), 3.04 (br dd, J = 4.7, 2.1 Hz, 1H), 2.91-2.75 (m, 1H), |
| | | | 2.20 (br t, J = 11.8 Hz, 1H), 1.96-1.83 (m, 4H), 1.79-1.67 (m, |
| | | | 4H), 1.34-1.14 (m, 4H) |
| 92 | 437 | 438 | δ 12.09-11.23 (m, 1 H) 8.44 (br s, 1 H), 8.17 (br s, 1 H), 7.94 |
| | | | (br d, J = 5.8 Hz, 1 H), 7.81 (br d, J = 6.1 Hz, 1 H), 7.47 (br s, 1 |
| | | | H), 7.70-7.53 (m, 3 H), 7.08-6.59 (m, 1 H), 6.44 (br d, J = 8.3 |
| | | | Hz, 1 H), 3.74-3.60 (m, 1 H), 2.55 (br d, J = 4.4 Hz, 3 H), 2.10- |
| | | | 1.93 (m, 3 H), 1.75 (br d, J = 12.1 Hz, 2 H), 1.51-1.35 (m, 2 H), |
| | | | 1.33-1.21 (m, 2 H), |
| 93 | 437 | 438 | δ 8.44 (s, 1 H) 7.85 (d, J-7.9 Hz, 1 H), 7.75 (s, 1 H), 7.73-7.67 |
| | | | (m, 2 H), 7.66-7.62 (m, 1 H), 7.60-7.50 (m, 2 H), 7.46 (br d, |
| | | | J = 6.0 Hz, 1 H), 6.51 (d, J = 9.1 Hz, 1 H), 6.34 (m, 1 H), 3.77 (br |
| | | | s, 1 H), 3.65 (br d, J = 2.3 Hz, 1 H), 1.80 (s, 3 H), 1.76-1.57 |
| | | | (m, 6 H), 1.56-1.47 (m, 2 H), |
| 94 | 409 | 410 | δ 8.42 (s, 1H), 7.91-7.82 (m, 2H), 7.77-7.62 (m, 4H), 7.60- |
| | | | 7.49 (m, 2H), 6.51 (d, J = 9.1 Hz, 1H), 6.34 (t, J = 6.6 Hz, 1H), |
| | | | 4.27-4.13 (m, 1H), 2.62 (br t, J = 7.9 Hz, 1H), 2.55 (d, J = 4.6 |
| | | | Hz, 3H), 2.36 (dq, J = 2.5, 7.8 Hz, 2H), 2.10 (q, J = 9.3 Hz, 2H) |
| 95 | 455 | 456 | δ 8.32 (br s, 1 H) 8.28-8.18 (m, 2 H) 7.55-7.47 (m, 2 H) 7.30 |
| | | | (t, J = 8.63 Hz, 2 H) 6.70 (d, J-9.63 Hz, 1 H) 3.76 (br t, J = 11.26 |
| | | | Hz, 1 H) 2.71 (s, 3 H) 2.25-2.03 (m, 3 H) 1.87 (br d, J = 11.88 |
| | | | Hz, 2 H) 1.59 (qd, J = 12.94, 2.94 Hz, 2 H) 1.40-1.22 (m, 2 H) |
| 96 | 401 | 402 | δ = 8.32 (br s, 1H), 8.26-8.08 (m, 1H), 7.99-7.82 (m, 1H), |
| | | | 7.71 (br d, J = 7.6 Hz, 1H), 7.36 (br d, J = 7.5 Hz, 1H), 6.47 (br |
| | | | d, J = 9.4 Hz, 1H), 3.71-3.56 (m, 1H), 3.53-3.37 (m, 2H), |
| | | | 1.99-1.87 (m, 2H), 1.86-1.72 (m, 5H), 1.39-1.18 (m, 4H), |
| | | | 1.11-1.00 (m, 2H), 0.93-0.81 (m, 2H). |
| 97 | 346 | 347 | δ = 8.49 (s, 1H), 8.25 (s, 1H), 8.18 (s, 1H), 4.67-4.56 (m, 1H), |
| | | | 3.76 (td, J = 3.6, 7.4 Hz, 1H), 3.03 (br dd, J = 4.4, 5.1 Hz, 1H), |
| | | | 2.75 (s, 3H), 2.65-2.56 (m, 2H), 2.33-2.22 (m, 2H), 1.22- |
| | | | 1.04 (m, 4H) |
| 98 | 360 | 361 | δ 8.51 (s, 1 H) 8.24 (d, J = 9.13 Hz, 2 H) 4.38-4.53 (m, 1 H) |
| | | | 4.05-4.17 (m, 1 H) 3.95 (br dd, J = 13.88, 1.75 Hz, 1 H) 3.78 |
| | | | (dt, J = 7.44, 3.53 Hz, 1 H) 3.26-3.32 (m, 1 H) 2.82-3.06 (m, 1 |
| | | | H) 2.14 (s, 3 H) 1.99-2.13 (m, 2 H) 1.41-1.63 (m, 2 H) 1.05- |
| | | | 1.23 (m, 4 H) |
| 99 | 374 | 375 | δ 8.47 (s, 1 H), 8.20 (d, J = 7.8 Hz, 2 H), 3.96 (br s, 1 H), 3.85- |
| | | | 3.73 (m, 2 H), 1.95 (s, 3 H), 1.81-1.67 (m, 8 H), 1.16-1.07 |
| | | | (m, 4 H) |
| 100 | 419 | 420 | δ 8.24 (br d, J = 2.5 Hz, 1H), 7.72 (br d, J = 7.5 Hz, 1H), 7.38- |
| | | | 7.15 (m, 1H), 4.51-4.35 (m, 1H), 4.34-4.20 (m, 1H), 3.66- |
| | | | 3.54 (m, 1H), 3.52-3.42 (m, 1H), 3.11-2.98 (m, 1H), 2.96- |
| | | | 2.82 (m, 1H), 1.93 (br dd, J = 11.9, 9.8 Hz, 5H), 1.84-1.67 (m, |
| | | | 6H), 1.64-1.43 (m, 1H), 1.42-1.14 (m, 5H), 0.79-0.61 (m, |
| | | | 4H) |
| 101 | 438 | 439 | δ 8.32 (s, 1 H), 7.99 (s, 1 H), 7.77 (br d, J-7.6 Hz, 1 H), 7.72 |
| | | | (d, J = 7.9 Hz, 1 H), 7.70-7.65 (m, 2 H), 7.59-7.53 (m, 1 H), |
| | | | 7.20 (t, J = 8.8 Hz, 2 H), 3.87-3.74 (m, 1 H), 2.71 (s, 3 H), 2.21- |
| | | | 2.14 (m, 3 H), 1.89 (br d, J = 12.4 Hz, 2 H), 1.66-1.57 (m, 2 |
| | | | H), 1.34 (br dd, J = 12.4, 2.8 Hz, 2 H) |
| 102 | 437 | 438 | δ 8.43 (br s, 1 H) 7.86 (d, J = 7.75 Hz, 1 H) 7.80-7.62 (m, 4 H) |
| | | | 7.60-7.39 (m, 3 H) 6.35 (t, J = 6.63 Hz, 1 H) 3.75-3.58 (m, 1 |
| | | | H) 3.47 (br dd, J = 7.44, 3.56 Hz, 1 H) 1.94 (br d, J = 10.38 Hz, |
| | | | 2H) 1.86-1.65 (m, 5 H) 1.43-1.12 (m, 4 H) |
| 103 | 437 | 438 | δ 8.43 (br s, 1 H) 7.85 (d, J-7.8 Hz, 1 H), 7.75 (br s, 1 H), 7.72- |
| | | | 7.61 (m, 3 H), 7.60-7.43 (m, 3 H), 6.50 (d, J = 9.1 Hz, 1 H), |
| | | | 6.34 (t, J = 6.6 Hz, 1 H), 3.65 (m, 1 H), 2.54 (d, J = 4.4 Hz, 3 H), |
| | | | 2.08-1.89 (m, 3 H), 1.73 (br d, J = 11.8 Hz, 2 H), 1.51-1.33 |
| | | | (m, 2 H), 1.31-1.17 (m, 2 H), |
| 104 | 437 | 438 | δ 11.88 (br s, 1H), 8.93 (s, 1H), 8.39-8.31 (m, 2H), 8.09-7.96 |
| | | | (m, 2H), 7.63 (br d, J = 4.6 Hz, 1H), 7.28-7.10 (m, 1H), 6.52 |
| | | | (br d, J = 9.6 Hz, 1H), 4.00-3.87 (m, 1H), 2.57 (d, J = 4.5 Hz, |
| | | | 3H), 2.26-2.15 (m, 1H), 1.89-1.76 (m, 4H), 1.63-1.47 (m, |
| | | | 4H) |
| 105 | 425 | 426 | δ 8.82 (br d, J = 4.88 Hz, 1 H) 8.26-8.13 (m, 3 H) 8.53 (br s, 1 |
| | | | H) 7.86-7.57 (m, 2 H) 7.35 (t, J-8.76 Hz, 2 H) 4.26 (br s, 1 H) |
| | | | 4.04-3.91 (m, 1 H) 3.81 (br d, J = 12.63 Hz, 1 H) 3.22-3.08 |
| | | | (m, 2 H) 2.80-2.65 (m, 1 H) 2.00 (s, 3 H) 1.97-1.83 (m, 2 H) |
| | | | 1.53-1.39 (m, 1 H) 1.38-1.26 (m, 1 H) |
| 106 | 401 | 402 | δ 8.34 (br s, 1H), 8.24 (s, 1H), 8.16-8.05 (m, 1H), 6.59 (d, J = |
| | | | 9.5 Hz, 1H), 3.83-3.69 (m, 1H), 3.49-3.38 (m, 1H), 2.71 (s, |
| | | | 3H), 2.24-2.17 (m, 1H), 2.13 (br d, J = 12.4 Hz, 2H), 1.89 (br |
| | | | d, J = 12.1 Hz, 2H), 1.61 (dq, J = 2.9, 12.9 Hz, 2H), 1.39-1.27 |
| | | | (m, 2H), 1.24-1.11 (m, 2H), 1.03-0.89 (m, 2H) |
| 107 | 374 | 375 | δ = 8.48 (s, 1H), 8.21 (d, J = 11.5 Hz, 2H), 4.08 (br s, 1H), 3.76 |
| | | | (td, J = 3.6, 7.4 Hz, 1H), 2.72 (s, 3H), 2.38-2.26 (m, 1H), 1.97- |
| | | | 1.81 (m, 5H), 1.78-1.61 (m, 5H), 1.19-1.07 (m, 4H) |
| 108 | 416 | 417 | δ = 8.45 (s, 1H), 8.25 (s, 1H), 8.18 (s, 1H), 4.29-4.15 (m, 1H), |
| | | | 4.07 (br s, 1H), 2.72 (s, 3H), 2.37-2.26 (m, 1H), 2.12 (br d, J = |
| | | | 10.8 Hz, 2H), 1.95-1.63 (m, 14H), 1.56-1.43 (m, 2H), 1.39- |
| | | | 1.24 (m, 2H) |
| 109 | 402 | 403 | δ = 8.31 (s, 1H), 7.77 (d, J = 2.4 Hz, 1H), 6.99 (d, J = 2.4 Hz, |
| | | | 1H), 4.48-4.39 (m, 1H), 4.32-4.22 (m, 1H), 4.18-4.07 (m, |
| | | | 1H), 3.99-3.89 (m, 1H), 2.98-2.89 (m, 1H), 2.21-2.09 (m, |
| | | | 6H), 2.08-2.02 (m, 1H), 1.98-1.91 (m, 2H), 1.88-1.79 (m, |
| | | | 2H), 1.52 (br d, J = 12.9 Hz, 4H), 1.35 (br d, J = 12.8 Hz, 1H) |
| 110 | 413 | 414 | δ = 11.96 (br d, J = 2.4 Hz, 1H), 8.94 (s, 1H), 8.36 (br s, 2H), |
| | | | 8.09-7.94 (m, 2H), 7.39 (br d, J = 7.6 Hz, 1H), 6.53 (d, J = 9.6 |
| | | | Hz, 1H), 4.29 (br d, J = 13.3 Hz, 1H), 4.12-3.92 (m, 1H), 3.82 |
| | | | (br d, J = 13.8 Hz, 1H), 3.18 (br s, 1H), 2.87-2.65 (m, 1H), |
| | | | 2.01 (s, 3H), 1.97-1.83 (m, 2H), 1.60-1.26 (m, 2H) |
| 111 | 413 | 414 | δ = 12.64-12.13 (m, 1H), 9.67 (s, 1H), 8.42 (br s, 1H), 8.35 (s, |
| | | | 1H), 8.15 (dd, J = 1.6, 7.4 Hz, 1H), 7.49 (dd, J = 1.6, 6.3 Hz, |
| | | | 1H), 7.42 (br d, J = 7.4 Hz, 1H), 6.44 (t, J = 6.9 Hz, 1H), 4.29 |
| | | | (br d, J = 13.4 Hz, 1H), 3.97 (br dd, J = 3.2, 4.9 Hz, 1H), 3.82 |
| | | | (br d, J = 13.5 Hz, 1H), 3.22-3.07 (m, 1H), 2.83-2.69 (m, |
| | | | 1H), 2.01 (s, 3H), 1.98-1.83 (m, 2H), 1.50-1.24 (m, 2H). |
| 112 | 407 | 408 | δ 9.79-9.59 (m, 1H), 8.92-8.82 (m, 1H), 8.49-8.33 (m, 1H), |
| | | | 8.05-7.91 (m, 1H), 7.68-7.58 (m, 1H), 7.28 (br s, 1H), 7.24- |
| | | | 7.08 (m, 1H), 7.06-6.76 (m, 1H), 6.69 (br s, 1H), 6.45 (br d, J = |
| | | | 8.9 Hz, 1H), 4.97-4.62 (m, 1H), 3.80-3.57 (m, 1H), 2.32- |
| | | | 2.16 (m, 1H), 2.07-1.87 (m, 2H), 1.82-1.64 (m, 2H), 1.45- |
| | | | 1.17 (m, 4H) |
| 113 | 386 | 387 | δ 8.34 (d, J = 3.4 Hz, 1H), 7.91 (d, J = 2.4 Hz, 1H), 7.22 (br s, |
| | | | 1H), 7.11 (br d, J = 7.3 Hz, 1H), 6.80 (s, 1H), 6.67 (br s, 1H), |
| | | | 4.31-4.18 (m, 1H), 3.77-3.64 (m, 1H), 2.21 (br s, 1H), 2.10- |
| | | | 1.62 (m, 11H), 1.49-1.11 (m, 7H) |
| 114 | 425 | 426 | δ 8.50 (br s, 1 H) 8.30-8.17 (m, 2 H) 8.14-8.02 (m, 2 H) 7.92- |
| | | | 7.57 (m, 2 H) 7.35 (br t, J = 8.38 Hz, 2 H) 4.35-4.17(m, 1 H) |
| | | | 4.05- 3.90 (m, 1 H) 3.81 (br d, J = 13.01 Hz, 1 H) 3.15 (br |
| | | | t, J = 11.88 Hz, 1 H) 2.74 (br t, J = 11.69 Hz, 1 H) 2.00 (s, 3 H) |
| | | | 1.97-1.84 (m, 2 H) 1.53-1.25 (m, 2 H) |
| 115 | 411 | 412 | δ = 8.47 (br s, 1H), 8.23 (br t, J = 6.1 Hz, 2H), 8.13-8.03 (m, |
| | | | 2H), 7.96-7.62 (m, 3H), 7.35 (br t, J = 8.4 Hz, 2H), 4.29-4.17 |
| | | | (m, 1H), 2.70-2.59 (m, 1H), 2.56 (br d, J = 4.1 Hz, 3H), 2.39 |
| | | | (br d, J = 7.6 Hz, 2H), 2.13 (br d, J = 9.5 Hz, 2H) |
| 116 | 374 | 375 | δ 8.48 (s, 1 H) 8.19 (br d, J = 11.88 Hz, 2 H) 3.81-3.60 (m, 3 H) |
| | | | 2.09 (br s, 2 H) 2.01-1.90 (m, 5 H) 1.45-1.32 (m, 4 H) 1.20- |
| | | | 1.04 (m, 4 H) |
| 117 | 374 | 375 | δ 8.48 (s, 1 H), 8.21 (d, J = 11.5 Hz, 2 H), 4.08 (br s, 1 H), 3.75 |
| | | | (tt, J = 7.3, 3.7 Hz, 1 H), 2.37-2.24 (m, 1 H), 1.96-1.84 (m, 4 |
| | | | H), 1.79-1.58 (m, 5 H), 1.18-1.08 (m, 4 H) |
| 118 | 416 | 417 | δ 8.46 (s, 1 H), 8.25 (s, 1 H), 8.18 (s, 1 H), 4.30-4.14 (m, 1 H), |
| | | | 3.87-3.70 (m, 1 H), 2.72 (s, 3 H), 2.14 (br d, J = 11.9 Hz, 5 H), |
| | | | 1.95-1.77 (m, 7 H), 1.64 (br d, J = 13.1 Hz, 2 H), 1.51 (br d, |
| | | | J = 12.6 Hz, 2 H), 1.36-1.28 (m, 3 H) |
| 119 | 399 | 400 | δ = 12.03-11.81 (m, 1H), 8.90 (s, 1H), 8.32 (br d, J = 7.4 Hz, |
| | | | 2H), 8.07-7.96 (m, 2H), 7.67 (br d, J = 4.1 Hz, 2H), 6.52 (br d, |
| | | | J = 9.6 Hz, 1H), 4.54-4.41 (m, 1H), 2.89 (br s, 1H), 2.59 (br d, |
| | | | J = 4.3 Hz, 3H), 2.42 (br s, 2H), 2.24-2.12 (m, 2H). |
| 120 | 413 | 414 | δ = 11.74 (br s, 1H), 9.17 (s, 1H), 8.39 (br s, 2H), 7.57 (br d, J = |
| | | | 7.0 Hz, 1H), 7.46 (br d, J = 7.5 Hz, 1H), 6.90 (br s, 2H), 4.29 |
| | | | (br d, J = 12.3 Hz, 1H), 4.16-3.93 (m, 1H), 3.82 (br d, J = 13.4 |
| | | | Hz, 1H), 3.19 (br d, J = 1.8 Hz, 1H), 2.77 (br d, J = 5.1 Hz, |
| | | | 1H), 2.01 (s, 3H), 1.98-1.83 (m, 2H), 1.62-1.19 (m, 2H) |
| 121 | 428 | 429 | δ 9.08 (s, 1 H) 8.37 (br d, J = 11.13 Hz, 2 H) 7.98 (br d, J = 3.13 |
| | | | Hz, 2 H) 7.76 (br d, J-7.50 Hz, 1 H) 7.40 (br t, J-8.57 Hz, 2 H) |
| | | | 7.27 (br d, J = 5.63 Hz, 1 H) 3.83-3.59 (m, 1 H) 3.49 (br s, 1 H) |
| | | | 1.95 (br s, 2 H) 1.87-1.68 (m, 5 H) 1.44-1.16 (m, 4 H) |
| 122 | 400 | 401 | δ = 9.15 (br s, 1H), 8.56-8.27 (m, 2H), 8.01 (br s, 2H), 7.80- |
| | | | 7.57 (m, 2H), 7.40 (br t, J = 8.6 Hz, 2H), 4.25 (br s, 1H), 2.64 |
| | | | (br d, J = 7.6 Hz, 1H), 2.57 (br d, J = 3.6 Hz, 3H), 2.38 (br d, J = |
| | | | 6.4 Hz, 2H), 2.30-2.12 (m, 2H) |
| 123 | 400 | 401 | δ = 9.05 (s, 1H), 8.36 (br d, J = 5.6 Hz, 2H), 7.97 (br dd, J = |
| | | | 4.5, 8.1 Hz, 2H), 7.77-7.59 (m, 2H), 7.39 (br t, J = 8.5 Hz, |
| | | | 2H), 4.58-4.39 (m, 1H), 2.90 (br s, 1H), 2.60 (br d, J = 4.0 Hz, |
| | | | 3H), 2.43 (br s, 2H), 2.26-2.11 (m, 2H). |
| 124 | 473 | 474 | δ 8.38-8.08 (m, 1H), 7.85-7.71 (m, 1H), 7.59-7.43 (m, 2H), |
| | | | 7.29 (br s, 3H), 4.67-4.26 (m, 1H), 3.76-3.41 (m, 3H), 3.22- |
| | | | 2.98 (m, 2H), 2.06-1.70 (m, 11H), 1.68-1.51 (m, 1H), 1.36- |
| | | | 1.18 (m, 4H) |
| 125 | 459 | 460 | δ 8.31-8.13 (m, 1H), 7.52-7.38 (m, 3H), 7.27 (br t, J = 8.2 |
| | | | Hz, 2H), 4.57-4.39 (m, 1H), 4.31-4.22 (m, 1H), 3.95-3.72 |
| | | | (m, 3H), 3.68-3.56 (m, 1H), 3.20-2.99 (m, 4H), 2.77-2.63 |
| | | | (m, 2H), 1.95-1.68 (m, 5H), 1.63-1.53 (m, 1H), 1.49-1.19 |
| | | | (m, 3H) |
| 126 | 459 | 460 | δ 8.31-8.12 (m, 1H), 7.74 (br d, J = 15.9 Hz, 1H), 7.64-7.54 |
| | | | (m, 2H), 7.34-7.17 (m, 3H), 3.85-3.64 (m, 3H), 3.63-3.41 |
| | | | (m, 4H), 2.37-2.17 (m, 1H), 2.13-1.98 (m, 1H), 1.89 (br d, J = |
| | | | 11.3 Hz, 2H), 1.78 (br s, 3H), 1.76 (br s, 2H), 1.39-1.06 (m, 4H) |
| 127 | 455 | 446 | δ 8.34-8.20 (m, 1H), 7.62 (br t, J = 6.6 Hz, 2H), 7.54-7.39 |
| | | | (m, 1H), 7.29 (br t, J = 7.6 Hz, 2H), 4.34-4.02 (m, 2H), 3.93- |
| | | | 3.69 (m, 4H), 3.69-3.49 (m, 2H), 3.23-2.97 (m, 1H), 2.66 (br |
| | | | d, J = 12.5 Hz, 1H), 2.37-2.20 (m, 1H), 2.16-2.05 (m, 1H), |
| | | | 2.04-1.97 (m, 3H), 1.94-1.75 (m, 2H), 1.48-1.23 (m, 2H) |
| 128 | 424 | 425 | δ 8.43 (s, 1H), 8.00 (s, 1H), 7.80-7.70 (m, 4H), 7.64-7.56 (m, |
| | | | 1H), 7.32-7.25 (m, 2H), 4.43-3.57 (m, 3H), 2.91-2.72 (m, |
| | | | 1H), 2.07-1.85 (m, 5H), 1.83-1.71 (m, 1H), 1.70-1.56 (m, |
| | | | 1H), 1.54-1.33 (m, 1H) |
| 129 | 423 | 424 | δ 8.38-8.31 (m, 1H), 8.02-7.84 (m, 2H), 7.68-7.58 (m, 3H), |
| | | | 7.53 (br d, J = 7.8 Hz, 1H), 6.64 (d, J = 9.5 Hz, 1H), 6.48 (t, J = |
| | | | 6.8 Hz, 1H), 4.30 (br d, J = 13.3 Hz, 1H), 4.10-4.01 (m, 1H), |
| | | | 3.96-3.81 (m, 1H), 3.73-3.62 (m, 1H), 3.28-2.95 (m, 2H), |
| | | | 2.12-1.96 (m, 4H), 1.89-1.48 (m, 3H) |
| 130 | 439 | 440 | δ 8.53 (s, 1 H) 8.22-8.32 (m, 2 H) 8.03-8.19 (m, 2 H) 7.70- |
| | | | 7.88 (m, 2 H) 7.51-7.62 (m, 1 H) 7.40 (t, J = 8.88 Hz, 2 H) 3.79- |
| | | | 3.90 (m, 1 H) 3.65-3.77 (m, 1 H) 1.86 (s, 3 H) 1.64-1.82 |
| | | | (m, 6 H) 1.54-1.63 (m, 2 H) |
| 131 | 439 | 440 | δ = 8.48 (s, 1H), 8.23 (dd, J = 5.7, 8.7 Hz, 2H), 8.13-8.04 (m, |
| | | | 2H), 7.86-7.74 (m, 1H), 7.61 (br d, J = 4.5 Hz, 1H), 7.53- |
| | | | 7.48 (m, 1H), 7.35 (t, J = 8.9 Hz, 2H), 3.95-3.87 (m, 1H), 2.56 |
| | | | (d, J = 4.5 Hz, 3H), 2.19 (td, J = 4.2, 8.3 Hz, 1H), 1.92-1.74 |
| | | | (m, 4H), 1.64-1.54 (m, 2H), 1.53-1.46 (m, 2H) |
| 132 | 360 | 361 | δ = 8.31 (br s, 1 H) 6.91 (br s, 1 H) 4.37-4.50 (m, 1 H) 4.05- |
| | | | 4.32 (m, 1 H) 3.94 (br d, J = 13.76 Hz, 1 H) 3.24-3.31 (m, 1 H) |
| | | | 2.92 (br s, 1 H) 2.14 (s, 3 H) 1.96-2.11 (m, 3 H) 1.35-1.57 |
| | | | (m, 2 H) 0.99 (br s, 2 H) 0.78 (br d, J = 3.13 Hz, 2 H) |
| 133 | 399 | 400 | δ = 11.74 (br s, 1H), 9.14 (s, 1H), 8.38 (br d, J = 12.6 Hz, 2H), |
| | | | 7.80-7.65 (m, 2H), 7.56 (br d, J = 6.9 Hz, 1H), 6.94-6.83 (m, |
| | | | 2H), 4.57-4.42 (m, 1H), 2.90 (br s, 1H), 2.59 (br d, J = 2.6 Hz, |
| | | | 3H), 2.43 (br s, 2H), 2.24-2.12 (m, 2H). |
| 134 | 428 | 429 | δ 9.07 (s, 1 H) 8.38 (br dd, J = 19.45, 4.31 Hz, 2 H) 7.98 (br dd, |
| | | | J = 8.94, 4.69 Hz, 2 H) 7.67 (br d, J-4.63 Hz, 1 H) 7.40 (t, |
| | | | J = 8.76 Hz, 2 H) 7.26 (d, J = 8.00 Hz, 1 H) 3.91-3.56 (m, 1 H) |
| | | | 2.56 (d, J-4.50 Hz, 3 H) 2.12-1.90 (m, 3 H) 1.75 (br d, |
| | | | J = 11.38 Hz, 2 H) 1.61-1.37 (m, 2 H) 1.34-1.14 (m, 2 H) |
| 135 | 445 | 446 | δ 8.28-8.18 (m, 1H), 7.64-7.58 (m, 2H), 7.45-7.33 (m, 1H), |
| | | | 7.32-7.24 (m, 3H), 6.69 (br s, 1H), 3.85-3.75 (m, 2H), 3.74- |
| | | | 3.66 (m, 2H), 3.64-3.56 (m, 2H), 2.34-2.15 (m, 2H), 2.14- |
| | | | 1.98 (m, 2H), 1.96-1.83 (m, 2H), 1.78-1.66 (m, 2H), 1.39- |
| | | | 1.30 (m, 1H), 1.26-1.18 (m, 2H) |
| 136 | 423 | 424 | δ 8.43 (br s, 1H), 7.85 (d, J = 7.8 Hz, 1H), 7.77-7.63 (m, 3H), |
| | | | 7.60-7.51 (m, 3H), 7.25-7.18 (m, 1H), 6.68 (br s, 1H), 6.51 |
| | | | (d, J = 9.0 Hz, 1H), 6.35 (t, J = 6.7 Hz, 1H), 3.80-3.68 (m, |
| | | | 1H), 2.26-2.14 (m, 1H), 2.00-1.84 (m, 2H), 1.82-1.63 (m, |
| | | | 2H), 1.42-1.13 (m, 4H) |
| 137 | 423 | 424 | δ 11.85 (br s, 1H), 8.43 (s, 1H), 8.11 (br s, 1H), 7.85 (br d, J = |
| | | | 7.8 Hz, 1H), 7.75-7.64 (m, 2H), 7.61-7.48 (m, 2H), 7.43 (br |
| | | | d, J = 6.0 Hz, 1H), 6.33 (t, J = 6.7 Hz, 1H), 4.26 (br d, J = 12.9 |
| | | | Hz, 1H), 4.04-3.88 (m, 1H), 3.80 (br d, J = 13.6 Hz, 1H), 3.14 |
| | | | (br t, J = 11.5 Hz, 1H), 2.74 (br t, J = 11.5 Hz, 1H), 2.00 (s, |
| | | | 5H), 1.55-1.16 (m, 2H) |
| 138 | 437 | 438 | δ 11.85 (br s, 1H), 8.41 (s, 1H), 8.09 (br s, 1H), 7.84 (br d, J = |
| | | | 7.5 Hz, 1H), 7.72-7.64 (m, 2H), 7.59 (br d, J = 4.4 Hz, 1H), |
| | | | 7.54-7.47 (m, 1H), 7.40 (br dd, J = 6.3, 11.8 Hz, 2H), 6.32 (t, J = |
| | | | 6.7 Hz, 1H), 3.90 (br s, 1H), 2.56 (d, J = 4.4 Hz, 3H), 2.18 (br |
| | | | s, 1H), 1.90-1.73 (m, 4H), 1.62-1.45 (m, 4H) |
| 139 | 409 | 410 | δ 8.30 (s, 1H), 8.11 (s, 1H), 7.82-7.76 (m, 3H), 7.55-7.49 (m, |
| | | | 1H), 7.45 (dd, J = 1.9, 6.4 Hz, 1H), 6.52 (t, J = 6.7 Hz, 1H), |
| | | | 4.62-4.55 (m, 1H), 3.35 (s, 1H), 3.04 (td, J = 4.9, 9.5 Hz, 1H), |
| | | | 2.73 (s, 3H), 2.66-2.55 (m, 2H), 2.35-2.22 (m, 2H). |
| 140 | 411 | 412 | δ 8.48 (s, 1 H) 8.24 (dd, J = 8.63, 5.63 Hz, 2 H) 8.16-8.03 (m, 2 |
| | | | H) 8.01-7.89 (m, 1 H) 7.85-7.64 (m, 2 H) 7.42-7.28 (m, 2 |
| | | | H) 4.57-4.43 (m, 1 H) 2.87 (td, J = 9.16, 4.32 Hz, 1 H) 2.58 (d, |
| | | | J = 4.50 Hz, 3 H) 2.41 (dt, J = 8.41, 4.11 Hz, 2 H) 2.20 (br dd, |
| | | | J = 5.32, 3.06 Hz, 2 H). |
| 141 | 425 | 426 | δ 8.74 (d, J = 5.13 Hz, 1 H) 8.43 (br d, J = 16.26 Hz, 1 H) 8.24- |
| | | | 8.14 (m, 1 H) 8.09-8.01 (m, 2 H) 7.81-7.68 (m, 1 H) 7.29- |
| | | | 7.20 (m, 2 H) 4.10 (br d, J = 12.51 Hz, 1 H) 4.00-3.67 (m, 2 H) |
| | | | 3.19- 2.90 (m, 2 H) 2.15-2.04 (m, 3 H) 1.89-1.51 (m, 4 H) |
| 142 | 439 | 440 | δ 8.82 (br s, 1 H) 8.51 (br s, 1 H) 8.19 (br dd, J = 8.26, 5.63 Hz, |
| | | | 3 H) 7.83-7.50 (m, 3 H) 7.36 (br t, J-8.63 Hz, 2 H) 3.67 (br d, |
| | | | J = 3.00 Hz, 1 H) 3.54-3.41 (m, 1 H) 2.02-1.64 (m, 7 H) 1.47- |
| | | | 1.09 (m, 4 H) |
| 143 | 441 | 442 | δ 8.64-8.14 (m, 3 H) 7.51 (ddt, J = 6.72, 4.57, 2.36, 2.36 Hz, 2 |
| | | | H) 7.30 (td, J = 8.63, 3.50 Hz, 2 H) 6.71 (dd, J = 9.51, 5.13 Hz, 1 |
| | | | H) 4.59-4.27 (m, 1 H) 4.12-3.96 (m, 1 H) 3.90-3.80 (m, 1 |
| | | | H) 3.74 (br d, J = 13.38 Hz, 1 H) 3.23-2.82 (m, 2 H) 2.22-2.02 |
| | | | (m, 3 H) 2.00-1.76 (m, 2 H) 1.74-1.47 (m, 2 H) |
| 144 | 374 | 375 | δ 8.27 (br s, 1 H), 7.01-6.51 (m, 1 H), 4.06-3.67 (m, 1 H), |
| | | | 2.71 (s, 3 H), 2.21-2.10 (m, 3 H), 2.02-1.95 (m, 1 H), 1.88 |
| | | | (br d, J = 12.3 Hz, 2 H), 1.72-1.59 (m, 2 H), 1.34-1.24 (m, 2 |
| | | | H), 0.97 (br d, J = 1.1 Hz, 2 H), 0.76 (br d, J = 4.5 Hz, 2 H) |
| 145 | 427 | 428 | δ 12.11-11.65 (m, 1 H) 8.92 (s, 1 H) 8.33 (br s, 2 H) 8.12- |
| | | | 7.91 (m, 2 H) 7.75 (d, J-7.75 Hz, 1 H) 7.23 (br d, J-2.75 Hz, 1 |
| | | | H) 6.52 (d, J = 9.63 Hz, 1 H) 3.85-3.60 (m, 1 H) 3.49 (br dd, |
| | | | J = 7.13, 3.50 Hz, 1 H) 1.94 (br s, 2 H) 1.86-1.68 (m, 5 H) 1.42- |
| | | | 1.15 (m, 4 H) |
| 146 | 427 | 428 | δ 12.69-11.38 (m, 1 H) 8.92 (s, 1 H) 8.44-8.22 (m, 2 H) 8.14- |
| | | | 7.97 (m, 2 H) 7.67 (br d, J = 4.38 Hz, 1 H) 7.24 (br d, J = 4.00 |
| | | | Hz, 1 H) 6.52 (d, J = 9.76 Hz, 1 H) 3.78-3.62 (m, 1 H) 2.55 (d, |
| | | | J = 4.50 Hz, 3 H) 2.13-1.87 (m, 3 H) 1.75 (br d, J = 11.76 Hz, 2 |
| | | | H) 1.61-1.40 (m, 2 H) 1.34-1.17 (m, 2 H) |
| 147 | 427 | 428 | δ 11.91-11.47 (m, 1 H) 9.16 (s, 1 H) 8.50-8.21 (m, 2 H) 7.75 |
| | | | (d, J = 7.75 Hz, 1 H) 7.56 (d, J = 7.13 Hz, 1 H) 7.30 (br d, J = 7.63 |
| | | | Hz, 1 H) 6.90 (br d, J = 6.63 Hz, 2 H) 3.84-3.62 (m, 1 H) 3.49 |
| | | | (br dd, J = 7.38, 3.38 Hz, 1 H) 1.94 (br d, J = 4.50 Hz, 2 H) 1.86- |
| | | | 1.73 (m, 5 H) 1.38-1.19 (m, 4 H) |
| 148 | 427 | 428 | δ = 11.75 (br s, 1H), 9.17 (s, 1H), 8.45 (s, 1H), 8.37 (s, 1H), |
| | | | 7.63 (br d, J = 4.5 Hz, 1H), 7.56 (d, J = 7.1 Hz, 1H), 7.24 (br d, |
| | | | J = 10.8 Hz, 1H), 6.94-6.87 (m, 2H), 4.06-3.84 (m, 1H), 2.57 |
| | | | (d, J = 4.5 Hz, 3H), 2.25-2.16 (m, 1H), 1.91-1.76 (m, 4H), |
| | | | 1.64-1.47 (m, 4H) |
| 149 | 427 | 428 | δ = 12.18-12.77 (m, 1 H) 9.65 (s, 1 H) 8.40 (s, 1 H) 8.30- |
| | | | 8.38 (m, 1 H) 8.07-8.21 (m, 1 H) 7.74 (br d, J = 7.63 Hz, 1 H) |
| | | | 7.43-7.59 (m, 1 H) 7.28 (br d, J-7.75 Hz, 1 H) 6.45 (t, J-6.88 |
| | | | Hz, 1 H) 3.61-3.73 (m, 1 H) 3.49 (br s, 1 H) 1.81-2.06 (m, 4 |
| | | | H) 1.79 (s, 3 H) 1.24-1.41 (m, 4 H). |
| 150 | 407 | 408 | δ 11.91-11.83 (m, 1H), 8.45-8.40 (m, 1H), 8.39-8.30 (m, |
| | | | 1H), 7.93-7.84 (m, 2H), 7.74-7.68 (m, 1H), 7.55 (t, J = 7.8 |
| | | | Hz, 1H), 7.46-7.40 (m, 1H), 7.23 (br d, J = 6.1 Hz, 2H), 6.67 |
| | | | (br s, 1H), 6.34 (t, J = 6.7 Hz, 1H), 3.79-3.66 (m, 1H), 2.24 (br |
| | | | t, J = 11.8 Hz, 1H), 2.01 (br dd, J = 2.7, 9.9 Hz, 1H), 1.97-1.89 |
| | | | (m, 1H), 1.82-1.65 (m, 2H), 1.40-1.17 (m, 4H) |
| 152 | 423 | 424 | δ 8.34 (br d, J = 14.1 Hz, 1H), 8.15-8.10 (m, 1H), 7.84-7.77 |
| | | | (m, 3H), 7.53 (t, J = 7.8 Hz, 1H), 7.47-7.42 (m, 1H), 6.52 (t, J = |
| | | | 6.7 Hz, 1H), 4.34-4.27 (m, 1H), 4.16-4.04 (m, 1H), 3.99- |
| | | | 3.91 (m, 1H), 3.90-3.77 (m, 1H), 3.74-3.66 (m, 1H), 3.16- |
| | | | 2.93 (m, 2H), 2.13-1.95 (m, 4H), 1.88-1.76 (m, 1H), 1.75- |
| | | | 1.63 (m, 1H), 1.62-1.46 (m, 1H) |
| 153 | 437 | 438 | δ 11.85 (br s, 1 H), 8.41 (s, 1 H), 8.10 (br s, 1 H), 7.84 (br d, |
| | | | J = 7.9 Hz, 1 H), 7.72-7.67 (m, 3 H), 7.53-7.49 (m, 1 H), 7.40 |
| | | | (br dd, J = 11.1, 6.5 Hz, 2 H), 6.33-6.30 (m, 1 H), 3.78-3.59 |
| | | | (m, 3 H), 1.80 (s, 4 H), 1.64 (br d, J = 9.4 Hz, 6 H) |
| 154 | 437 | 438 | δ 11.91 (br d, J = 1.50 Hz, 1 H) 8.47 (br s, 1 H) 8.15 (br s, 1 H) |
| | | | 7.91 (br d, J-7.75 Hz, 1 H) 7.85-7.68 (m, 3 H) 7.62-7.54 (m, |
| | | | 1 H) 7.49 (br d, J = 6.00 Hz, 2 H) 6.38 (t, J = 6.57 Hz, 1 H) 3.75- |
| | | | 3.64 (m, 1 H) 3.54 (br dd, J = 7.50, 3.75 Hz, 1 H) 2.01 (br d, |
| | | | J = 11.38 Hz, 2 H) 1.93-1.73 (m, 5 H) 1.51-1.17 (m, 4 H) |
| 155 | 437 | 438 | δ ppm 8.29 (s, 1 H), 8.11 (br s, 1 H), 7.76-7.83 (m, 3 H), 7.52 |
| | | | (t, J = 7.8 Hz, 1 H), 7.45 (br d, J = 6.5 Hz, 1 H), 7.13-7.04 (m, 1 |
| | | | H), 6.52 (t, J-6.6 Hz, 1 H), 3.86-3.72 (m, 1 H), 2.70 (s, 3 H), |
| | | | 2.22-2.10 (m, 4 H), 1.88 (br d, J-12.6 Hz, 2 H), 1.61 (q, |
| | | | J = 12.3 Hz, 2 H), 1.40-1.23 (m, 1 H) |
| 156 | 409 | 410 | δ 8.30 (s, 1H), 8.10 (s, 1H), 7.85-7.69 (m, 3H), 7.60-7.40 (m, |
| | | | 2H), 6.52 (t, J = 6.7 Hz, 1H), 4.38 (br t, J = 8.2 Hz, 1H), 2.80- |
| | | | 2.74 (m, 1H), 2.74-2.69 (m, 3H), 2.62-2.52 (m, 2H), 2.23- |
| | | | 2.12 (m, 2H) |
| 157 | 423 | 424 | δ 11.94-11.16 (m, 1H), 8.53-8.12 (m, 2H), 7.98-7.76 (m, |
| | | | 2H), 7.73-7.52 (m, 3H), 6.99-6.62 (m, 1H), 6.44 (br d, J = |
| | | | 8.5 Hz, 1H), 4.54-3.59 (m, 3H), 3.11-2.85 (m, 1H), 2.78- |
| | | | 2.57 (m, 1H), 2.06-1.89 (m, 3H), 1.87-1.67 (m, 2H), 1.67- |
| | | | 1.30 (m, 2H) |
| 158 | 439 | 440 | δ 8.47 (br s, 1 H) 8.23 (br t, J = 6.50 Hz, 2 H) 8.15-7.98 (m, 2 |
| | | | H) 7.90-7.64 (m, 2 H) 7.53 (br s, 1 H) 7.35 (br t, J-8.76 Hz, 2 |
| | | | H) 3.75-3.59 (m, 1 H) 3.56-3.41 (m, 1 H) 2.06-1.87 (m, 2 |
| | | | H) 1.86-1.79 (m, 2 H) 1.77 (s, 3 H) 1.42-1.16 (m, 4 H) |
| 159 | 439 | 440 | δ 8.47 (br s, 1 H) 8.23 (br s, 2 H) 8.02-8.12 (m, 2 H) 7.92- |
| | | | 7.71 (m, 1 H) 7.66 (br d, J = 4.50 Hz, 1 H) 7.53 (br d, J = 1.88 Hz, |
| | | | 1 H) 7.35 (br t, J = 8.82 Hz, 2 H) 3.76-3.62 (m, 1 H) 2.55 (br d, |
| | | | J = 4.38 Hz, 3 H) 2.11-1.94 (m, 3 H) 1.75 (br d, J = 12.38 Hz, 2 |
| | | | H) 1.51-1.37 (m, 2 H) 1.35-1.21 (m, 2 H) |
| 160 | 439 | 440 | δ 8.81 (br d, J = 1.00 Hz, 1 H) 8.51 (br s, 1 H) 8.26-8.11 (m, 3 |
| | | | H) 7.65 (br s, 3 H) 7.35 (br t, J = 8.76 Hz, 2 H) 3.75-3.57 (m, 1 |
| | | | H) 2.54 (br d, J = 3.25 Hz, 3 H) 2.09-1.91 (m, 3 H) 1.75 (br d, |
| | | | J = 11.51 Hz, 2 H) 1.42 (q, J = 12.72 Hz, 2 H) 1.34- 1.20 (m, 2 H) |
| 161 | 346 | 347 | δ = 8.27 (s, 1H), 6.83 (s, 1H), 4.45 (br s, 1H), 2.81-2.75 (m, |
| | | | 1H), 2.73 (s, 3H), 2.55 (dq, J = 2.4, 8.0 Hz, 2H), 2.35-2.21 (m, |
| | | | 2H), 2.05-1.94 (m, 1H), 1.02-0.93 (m, 2H), 0.82-0.72 (m, |
| | | | 2H) |
| 162 | 346 | 347 | δ = 8.27 (s, 1H), 6.98-6.79 (m, 1H), 3.00 (br s, 1H), 2.76 (s, |
| | | | 4H), 2.63-2.60 (m, 1H), 2.59-2.55 (m, 1H), 2.31-2.27 (m, |
| | | | 1H), 2.26-2.22 (m, 1H), 2.03-1.95 (m, 1H), 0.97 (br d, J = |
| | | | 6.8 Hz, 2H), 0.79-0.75 (m, 2H). |
| 163 | 427 | 428 | δ = 8.43 (s, 1H), 7.85 (br d, J = 7.8 Hz, 1H), 7.75 (s, 1H), 7.70 |
| | | | (br d, J = 6.3 Hz, 1H), 7.65 (t, J = 7.8 Hz, 1H), 7.56 (br d, J = |
| | | | 9.0 Hz, 1H), 7.45 (br d, J = 6.5 Hz, 1H), 6.51 (d, J = 9.3 Hz, |
| | | | 1H), 6.34 (dt, J = 1.0, 6.7 Hz, 1H), 3.89 (br s, 1H), 2.56 (d, J = |
| | | | 4.5 Hz, 3H), 2.18 (br s, 1H), 1.89-1.71 (m, 4H), 1.62-1.43 |
| | | | (m, 4H) |
| 164 | 399 | 400 | δ = 12.03-11.79 (m, 1H), 9.12-8.76 (m, 1H), 8.55-8.20 (m, |
| | | | 2H), 8.14-7.92 (m, 2H), 7.82-7.50 (m, 2H), 6.52 (d, J = 9.6 |
| | | | Hz, 1H), 4.24 (br d, J = 7.3 Hz, 1H), 2.69-2.61 (m, 1H), 2.57 |
| | | | (d, J = 4.6 Hz, 3H), 2.40-2.35 (m, 2H), 2.21 (br s, 2H) |
| 165 | 427 | 428 | δ 9.07 (s, 1 H) 8.38 (br dd, J = 19.45, 4.31 Hz, 2 H) 7.98 (br dd, |
| | | | J = 8.94, 4.69 Hz, 2 H) 7.67 (br d, J = 4.63 Hz, 1 H) 7.40 (t, |
| | | | J = 8.76 Hz, 2 H) 7.26 (d, J = 8.00 Hz, 1 H) 3.84-3.62 (m, 1 H) |
| | | | 2.56 (d, J = 4.50 Hz, 3 H) 2.12-1.90 (m, 3 H) 1.75 (br d, |
| | | | J = 11.38 Hz, 2 H) 1.63-1.39 (m, 2 H 1.35-1.17 (m, 2 H) |
| 166 | 399 | 400 | δ 12.21-12.66 (m, 1 H) 9.65 (s, 1 H) 8.39 (s, 1 H) 8.35 (s, 1 H) |
| | | | 8.16 (dd, J = 7.32, 1.69 Hz, 1 H) 7.63-7.80 (m, 2 H) 7.51 (dd, |
| | | | J = 6.38, 1.63 Hz, 1 H) 6.45 (t, J = 6.88 Hz, 1 H) 4.25-4.66 (m, 1 |
| | | | H) 2.80-2.99 (m, 1 H) 2.58-2.63 (m, 3 H) 2.35-2.39 (m, 2 |
| | | | H) 2.20 (br d, J = 8.50 Hz, 2 H) |
| 167 | 427 | 428 | δ = 9.32 (s, 1H), 8.45 (s, 1H), 8.25 (s, 1H), 7.79 (t, J = 7.9 Hz, |
| | | | 1H), 7.46 (d, J = 7.8 Hz, 1H), 6.63 (d, J = 8.1 Hz, 1H), 4.60 (s, |
| | | | 1H), 4.12 (br s, 1H), 2.73 (s, 3H), 2.38-2.27 (m, 1H), 2.09- |
| | | | 1.81 (m, 5H), 1.80-1.58 (m, 5H) |
| 168 | 441 | 442 | δ 8.43 (br s, 1H), 7.91 (br s, 1H), 7.86 (d, J = 7.9 Hz, 1H), 7.75 |
| | | | (br s, 1H), 7.67 (br t, J = 7.9 Hz, 1H), 7.60-7.49 (m, 2H), 7.22 |
| | | | (br s, 1H), 6.69 (br s, 1H), 6.49-6.42 (m, 1H), 6.35 (dd, J = |
| | | | 11.5, 2.8 Hz, 1H), 4.10-3.84 (m, 1H), 2.21 (br t, J = 11.7 Hz, |
| | | | 1H), 1.96 (br d, J = 12.4 Hz, 1H), 1.90 (br d, J = 10.5 Hz, 1H), |
| | | | 1.77 (br d, J = 12.0 Hz, 1H), 1.70 (br d, J = 11.9 Hz, 1H), 1.38- |
| | | | 1.16 (m, 4H) |
| 169 | 370 | 371 | δ 8.45 (d, J = 3.8 Hz, 1H), 8.02 (s, 1H), 7.87 (br d, J = 6.6 Hz, |
| | | | 1H), 7.62-7.52 (m, 2H), 7.34 (br d, J = 7.8 Hz, 1H), 5.03- |
| | | | 4.97 (m, 2H), 4.65 (t, J = 6.3 Hz, 2H), 4.40-4.24 (m, 2H), 4.00- |
| | | | 3.90 (m, 1H), 3.87-3.78 (m, 1H), 3.22-3.12 (m, 1H), 2.80- |
| | | | 2.67 (m, 1H), 2.02 (s, 3H), 1.99-1.86 (m, 2H), 1.51-1.28 (m, |
| | | | 2H) |
| 170 | 370 | 371 | δ 8.42 (d, J = 3.6 Hz, 1H), 8.05-7.98 (m, 1H), 7.86 (br d, J = |
| | | | 7.3 Hz, 1H), 7.61-7.51 (m, 2H), 7.29-7.19 (m, 2H), 6.72- |
| | | | 6.65 (m, 1H), 5.00 (dd, J = 8.3, 5.9 Hz, 2H), 4.69-4.62 (m, |
| | | | 2H), 4.40-4.29 (m, 1H), 3.79-3.67 (m, 1H), 2.28-2.17 (m, |
| | | | 1H), 2.04-1.90 (m, 2H), 1.84-1.68 (m, 2H), 1.40-1.28 (m, |
| | | | 2H), 1.27-1.17 (m, 2H) |
| 171 | 386 | 387 | δ 8.45 (d, J = 3.8 Hz, 1H), 8.27 (br s, 1H), 7.91 (br d, J = 7.4 |
| | | | Hz, 1H), 7.79 (d, J = 8.0 Hz, 1H), 7.58 (t, J = 7.8 Hz, 1H), 7.35 |
| | | | (br d, J = 7.3 Hz, 1H), 6.51 (s, 1H), 4.83 (d, J = 6.6 Hz, 2H), |
| | | | 4.71 (d, J = 6.6 Hz, 2H), 4.29 (br d, J = 12.5 Hz, 1H), 4.02- |
| | | | 3.88 (m, 1H), 3.83 (br d, J = 13.9 Hz, 1H), 3.15 (br t, J = 11.4 |
| | | | Hz, 1H), 2.79-2.68 (m, 1H), 2.01 (s, 3H), 2.00-1.88 (m, 2H), |
| | | | 1.51-1.27 (m, 2H) |
| 172 | 373 | 374 | δ 8.67 (d, J = 5.0 Hz, 1H), 8.54 (d, J = 3.5 Hz, 1H), 8.23 (s, |
| | | | 1H), 7.72 (br d, J = 3.8 Hz, 1H), 7.49 (br d, J = 7.6 Hz, 1H), |
| | | | 5.34 (s, 1H), 4.29 (br d, J = 13.5 Hz, 1H), 3.99-3.92 (m, 1H), |
| | | | 3.83 (br d, J = 13.5 Hz, 1H), 3.21-3.12 (m, 1H), 2.79-2.68 |
| | | | (m, 1H), 2.02 (s, 3H), 1.93 (br dd, J = 18.3, 14.7 Hz, 2H), 1.48 |
| | | | (s, 6H), 1.45-1.29 (m, 2H) |
| 173 | 386 | 387 | δ 8.44 (d, J = 3.7 Hz, 1H), 8.35-8.19 (m, 1H), 7.91 (br d, J = |
| | | | 7.0 Hz, 1H), 7.80 (d, J = 7.8 Hz, 1H), 7.63-7.55 (m, 1H), 7.37- |
| | | | 7.27 (m, 1H), 7.24 (br s, 1H), 6.73 (br s, 1H), 4.84 (d, J = 6.6 |
| | | | Hz, 2H), 4.72 (d, J = 6.6 Hz, 2H), 3.74 (br s, 1H), 3.57-3.49 |
| | | | (m, 1H), 2.23 (br t, J = 11.8 Hz, 1H), 2.00 (br d, J = 12.5 Hz, |
| | | | 2H), 1.83-1.76 (m, 1H), 1.72 (br d, J = 11.1 Hz, 1H), 1.42- |
| | | | 1.17 (m, 4H) |
| 174 | 453 | 454 | δ 8.42 (d, J = 3.3 Hz, 1H), 7.95-7.79 (m, 2H), 7.75 (br d, J = |
| | | | 7.5 Hz, 1H), 7.56-7.43 (m, 2H), 7.23-7.11 (m, 1H), 4.53- |
| | | | 4.41 (m, 1H), 3.94-3.80 (m, 1H), 3.74-3.60 (m, 1H), 3.59- |
| | | | 3.44 (m, 1H), 3.22-3.02 (m, 1H), 2.87-2.59 (m, 2H), 2.05 (d, |
| | | | J = 5.9 Hz, 3H), 2.03-1.94 (m, 3H), 1.87-1.74 (m, 7H), 1.64- |
| | | | 1.43 (m, 1H), 1.40-1.20 (m, 4H) |
| 175 | 384 | 385 | δ 8.42 (d, J = 3.7 Hz, 1H), 8.05-7.96 (m, 1H), 7.91-7.80 (m, |
| | | | 1H), 7.73 (br d, J = 7.8 Hz, 1H), 7.62-7.49 (m, 2H), 7.22- |
| | | | 7.13 (m, 1H), 5.06-4.93 (m, 2H), 4.70-4.59 (m, 2H), 4.43- |
| | | | 4.23 (m, 1H), 3.72-3.60 (m, 1H), 3.56-3.44 (m, 1H), 2.04- |
| | | | 1.92 (m, 2H), 1.80 (br s, 2H), 1.78 (s, 3H), 1.40-1.20 (m, 4H) |
| 176 | 400 | 401 | δ 8.45-8.38 (m, 1H), 8.26 (br s, 1H), 7.92-7.85 (m, 1H), 7.77 |
| | | | (br t, J = 7.2 Hz, 2H), 7.60-7.54 (m, 1H), 7.20 (br d, J = 7.4 |
| | | | Hz, 1H), 6.51 (s, 1H), 4.86-4.78 (m, 2H), 4.73-4.67 (m, 2H), |
| | | | 3.72-3.56 (m, 1H), 3.54-3.46 (m, 1H), 2.05-1.90 (m, 2H), |
| | | | 1.84-1.79 (m, 2H), 1.77 (s, 3H), 1.38-1.19 (m, 4H) |
| 177 | 373 | 374 | δ 8.67 (d, J = 5.0 Hz, 1H), 8.52 (d, J = 3.3 Hz, 1H), 8.24 (br s, |
| | | | 1H), 7.72 (br s, 1H), 7.45 (br d, J = 6.1 Hz, 1H), 7.25 (br s, 1H), |
| | | | 6.73 (br s, 1H), 5.38 (s, 1H), 3.80-3.69 (m, 1H), 2.23 (br t, J = |
| | | | 11.3 Hz, 1H), 2.04-1.89 (m, 2H), 1.79 (br d, J = 12.0 Hz, 1H), |
| | | | 1.71 (br d, J = 11.2 Hz, 1H), 1.48 (s, 6H), 1.38-1.18 (m, 4H) |
| 178 | 439 | 440 | δ 8.41 (d, J = 3.4 Hz, 1H), 7.90-7.78 (m, 2H), 7.53-7.44 (m, |
| | | | 2H), 7.27-7.19 (m, 2H), 6.68 (br s, 1H), 4.49-4.41 (m, 1H), |
| | | | 3.90-3.82 (m, 1H), 3.78-3.65 (m, 1H), 3.19-3.05 (m, 1H), |
| | | | 2.69-2.55 (m, 2H), 2.28-2.17 (m, 1H), 2.04 (d, J = 6.1 Hz, |
| | | | 3H), 1.97 (br d, J = 12.1 Hz, 2H), 1.83-1.68 (m, 5H), 1.64- |
| | | | 1.51 (m, 1H), 1.38-1.17 (m, 4H) |
| 179 | 439 | 440 | δ 8.44 (d, J = 3.6 Hz, 1H), 7.91-7.78 (m, 2H), 7.55-7.43 (m, |
| | | | 2H), 7.37-7.28 (m, 1H), 4.50-4.41 (m, 1H), 4.32-4.25 (m, |
| | | | 1H), 4.00-3.78 (m, 3H), 3.21-3.04 (m, 2H), 2.85-2.56 (m, |
| | | | 3H), 2.05-2.00 (m, 6H), 1.99-1.86 (m, 3H), 1.83-1.69 (m, |
| | | | 2H), 1.57-1.28 (m, 3H) |
| 180 | 370 | 371 | δ 8.42 (d, J = 3.8 Hz, 1H), 8.00 (s, 1H), 7.85 (br d, J = 6.1 Hz, |
| | | | 1H), 7.67-7.44 (m, 2H), 7.25-7.11 (m, 2H), 6.69 (br s, 1H), |
| | | | 4.99 (dd, J = 5.9, 8.3 Hz, 2H), 4.64 (t, J = 6.3 Hz, 2H), 4.33 |
| | | | (quin, J = 7.5 Hz, 1H), 3.71-3.54 (m, 1H), 2.15-1.92 (m, 3H), |
| | | | 1.79 (br d, J = 11.5 Hz, 2H), 1.50-1.35 (m, 2H), 1.34-1.20 |
| | | | (m, 2H) |
| 181 | 386 | 387 | δ 8.42 (d, J = 3.6 Hz, 1H), 8.26 (br s, 1H), 7.90 (br d, J = 7.1 |
| | | | Hz, 1H), 7.79 (d, J = 7.9 Hz, 1H), 7.64-7.50 (m, 1H), 7.20 (br |
| | | | s, 2H), 6.69 (br s, 1H), 4.83 (d, J = 6.6 Hz, 2H), 4.71 (d, J = 6.5 |
| | | | Hz, 2H), 3.65 (br s, 2H), 2.15-1.95 (m, 3H), 1.80 (br d, J = |
| | | | 11.8 Hz, 2H), 1.50-1.36 (m, 2H), 1.34-1.22 (m, 2H) |
| 182 | 337 | 338 | δ 7.84 (d, J = 6.8 Hz, 1H), 7.74-7.67 (m, 1H), 6.48 (br d, J = |
| | | | 6.1 Hz, 1H), 3.68-3.65 (m, 4H), 3.60-3.56 (m, 4H), 3.54- |
| | | | 3.41 (m, 2H), 1.92-1.86 (m, 2H), 1.81-1.75 (m, 5H), 1.30- |
| | | | 1.14 (m, 4H) |
| 187 | 407 | 408 | δ 11.79-11.53 (m, 1H), 8.49-8.47 (m, 1H), 8.24 (s, 1H), 8.04 |
| | | | (br d, J = 7.4 Hz, 1H), 7.87 (br d, J = 7.3 Hz, 1H), 7.69-7.64 |
| | | | (m, 1H), 7.50 (br d, J = 6.9 Hz, 1H), 7.38 (br d, J = 7.6 Hz, 1H), |
| | | | 6.62 (s, 1H), 6.54 (br d, J = 7.0 Hz, 1H), 4.32-4.25 (m, 1H), |
| | | | 3.99-3.91 (m, 1H), 3.82 (br d, J = 13.1 Hz, 1H), 3.20-3.13 |
| | | | (m, 1H), 2.79-2.73 (m, 1H), 2.01 (s, 3H), 1.97-1.88 (m, 2H), |
| | | | 1.50-1.41 (m, 1H), 1.38-1.30 (m, 1H) |
| 188 | 421 | 422 | δ 11.75-11.60 (m, 1H), 8.46 (d, J = 3.5 Hz, 1H), 8.26-8.23 |
| | | | (m, 1H), 8.03 (br d, J = 7.5 Hz, 1H), 7.87 (br d, J = 8.0 Hz, 1H), |
| | | | 7.74 (br d, J = 7.4 Hz, 1H), 7.66 (t, J = 7.8 Hz, 1H), 7.51 (d, J = |
| | | | 6.8 Hz, 1H), 7.24 (br d, J = 7.6 Hz, 1H), 6.63 (d, J = 1.3 Hz, |
| | | | 1H), 6.54 (br d, J = 6.0 Hz, 1H), 3.71-3.62 (m, 1H), 3.55- |
| | | | 3.45 (m, 1H), 1.98 (br d, J = 8.8 Hz, 2H), 1.85-1.80 (m, 2H), |
| | | | 1.78 (s, 3H), 1.40-1.31 (m, 2H), 1.31-1.21 (m, 2H) |
| 189 | 414 | 415 | δ 8.15 (s, 1H), 7.86 (d, J = 6.9 Hz, 1H), 7.81-7.77 (m, 1H), |
| | | | 7.45-7.36 (m, 1H), 6.68 (br d, J = 5.6 Hz, 1H), 6.41 (dd, J = |
| | | | 9.1, 2.7 Hz, 1H), 6.28 (t, J = 6.7 Hz, 1H), 4.82 (br s, 1H), 4.37 |
| | | | (br d, J = 11.8 Hz, 1H), 4.32-4.19 (m, 2H), 3.82-3.72 (m, |
| | | | 2H), 3.19 (br t, J = 11.6 Hz, 1H), 3.10-2.99 (m, 2H), 2.67- |
| | | | 2.54 (m, 1H), 2.05-1.99 (m, 1H), 1.98 (d, J = 4.8 Hz, 3H), |
| | | | 1.93-1.75 (m, 4H), 1.73-1.58 (m, 1H), 1.38-1.15 (m, 2H) |
| 190 | 374 | 375 | δ 8.27 (s, 1 H) 6.80 (br s, 1 H) 4.00-3.53 (m, 2 H) 2.22-1.76 |
| | | | (m, 8 H) 1.49-1.20 (m, 4 H) 0.98 (q, J-6.09 Hz, 2 H) 0.83- |
| | | | 0.69 (m, 2 H) |
| 191 | 374 | 375 | δ = 8.28 (s, 1H), 6.81 (s, 1H), 4.77-4.50 (m, 1H), 4.15 (br d, J = |
| | | | 3.3 Hz, 1H), 2.75-2.70 (m, 3H), 2.36-2.28 (m, 1H), 2.04- |
| | | | 1.95 (m, 1H), 1.94-1.80 (m, 5H), 1.77-1.63 (m, 5H), 1.03- |
| | | | 0.94 (m, 2H), 0.80-0.72 (m, 2H) |
| 192 | 360 | 361 | δ 8.40-8.28 (m, 1H), 6.99-6.62 (m, 1H), 4.11-3.72 (m, 3H), |
| | | | 3.29-3.07 (m, 2H), 2.19-2.09 (m, 3H), 2.06-1.97 (m, 2H), |
| | | | 1.93-1.82 (m, 1H), 1.76-1.63 (m, 2H), 0.99 (br s, 2H), 0.84- |
| | | | 0.75 (m, 2H) |
| 193 | 399 | 400 | δ = 8.54 (d, J = 1.5 Hz, 1H), 8.32-8.17 (m, 2H), 8.15-8.02 |
| | | | (m, 2H), 7.87 (br d, J = 6.0 Hz, 2H), 7.35 (br t, J = 8.1 Hz, 2H), |
| | | | 4.42-4.33 (m, 1H), 4.26 (br d, J = 13.0 Hz, 1H), 3.70 (br d, J = |
| | | | 3.3 Hz, 1H), 3.13-3.02 (m, 1H), 1.79 (s, 3H), 1.57-1.41 (m, |
| | | | 2H) |
| 194 | 425 | 426 | δ 8.54 (d, J = 1.50 Hz, 1 H) 8.32-8.19 (m, 2 H) 8.15-8.01 (m, |
| | | | 2 H) 7.87 (br d, J = 6.00 Hz, 2 H) 7.35 (br t, J = 8.07 Hz, 2 H) |
| | | | 4.45-4.18 (m, 2 H) 3.70 (br d, J-3.25 Hz, 1 H) 3.21 (br d, |
| | | | J = 1.00 Hz, 1 H) 3.13-3.02 (m, 1 H) 1.95-1.72 (m, 5 H) 1.56- |
| | | | 1.41 (m, 2 H) |
| 195 | 407 | 408 | δ 11.72-11.63 (m, 1H), 8.46 (d, J = 3.6 Hz, 1H), 8.26-8.23 |
| | | | (m, 1H), 8.04 (br d, J = 6.9 Hz, 1H), 7.87 (br d, J = 7.8 Hz, 1H), |
| | | | 7.67 (br t, J = 7.7 Hz, 1H), 7.50 (d, J = 6.6 Hz, 1H), 7.26-7.19 |
| | | | (m, 2H), 6.69 (br s, 1H), 6.63 (s, 1H), 6.54 (dd, J = 6.9, 1.13 |
| | | | Hz, 1H), 3.70-3.61 (m, 1H), 2.11-2.06 (m, 1H), 2.02 (br d, J = |
| | | | 13.0 Hz, 2H), 1.83-1.78 (m, 2H), 1.48-1.39 (m, 2H), 1.34- |
| | | | 1.24 (m, 2H) |
| 196 | 414 | 415 | δ 12.19-11.11 (m, 1H), 8.15 (s, 1H), 7.81 (d, J = 6.9 Hz, 1H), |
| | | | 7.31 (br d, J = 6.5 Hz, 1H), 7.25 (br d, J = 6.3 Hz, 1H), 6.57 (br |
| | | | d, J = 6.8 Hz, 1H), 6.16 (br t, J = 6.4 Hz, 1H), 4.42 (br d, J = |
| | | | 8.3 Hz, 1H), 4.34 (br d, J = 13.1 Hz, 1H), 4.29-4.18 (m, 1H), |
| | | | 3.84-3.71 (m, 2H), 3.10-3.01 (m, 1H), 2.98-2.82 (m, 3H), |
| | | | 2.64 (br t, J = 11.4 Hz, 1H), 2.02-1.95 (m, 3H), 1.93-1.72 (m, |
| | | | 5H), 1.64-1.52 (m, 1H), 1.40-1.16 (m, 2H) |
| 197 | 418 | 419 | δ 7.91 (br d, J = 4.0 Hz, 1H), 7.78 (d, J = 7.0 Hz, 1H), 7.69 (br |
| | | | d, J = 7.5 Hz, 1H), 6.46-6.40 (m, 1H), 4.29 (br d, J = 13.0 Hz, |
| | | | 1H), 4.23-4.18 (m, 1H), 3.49-3.42 (m, 2H), 2.94 (br s, 1H), |
| | | | 2.93-2.88 (m, 1H), 2.60 (dt, J = 7.3, 3.7 Hz, 1H), 2.26 (ddd, J = |
| | | | 11.0, 7.2, 3.6 Hz, 2H), 1.87 (br d, J =13.4 Hz, 3H), 1.76 (s, |
| | | | 3H), 1.69 (br d, J = 13.9 Hz, 1H), 1.64-1.58 (m, 1H), 1.43 (br |
| | | | dd, J = 11.8, 3.69 Hz, 1H), 1.28-1.14 (m, 5H), 0.60-0.57 (m, |
| | | | 2H), 0.36 (br d, J = 2.3 Hz, 2H) |
| 198 | 418 | 419 | δ 8.28 (br s, 1H), 7.93 (br d, J = 4.1 Hz, 1H), 7.78 (d, J = 6.9 |
| | | | Hz, 1H), 7.71 (br d, J = 7.9 Hz, 1H), 6.45 (br d, J = 5.0 Hz, |
| | | | 1H), 4.34-4.26 (m, 1H), 4.25-4.15 (m, 1H), 3.44 (td, J = 7.3, |
| | | | 3.8 Hz, 3H), 2.98-2.88 (m, 2H), 2.60 (dt, J = 7.3, 3.9 Hz, 1H), |
| | | | 2.26 (ddd, J = 11.0, 7.5, 3.5 Hz, 1H), 1.87 (br d, J = 12.6 Hz, |
| | | | 2H), 1.79 (br s, 2H), 1.76 (s, 3H), 1.71-1.59 (m, 2H), 1.50- |
| | | | 1.37 (m, 1H), 1.28-1.12 (m, 4H), 0.62-0.56 (m, 2H), 0.39- |
| | | | 0.32 (m, 2H) |
| 199 | 414 | 415 | δ 11.89-11.23 (m, 1H), 8.11 (d, J = 8.4 Hz, 1H), 8.04-7.80 |
| | | | (m, 1H), 7.45 (dd, J = 2.6, 9.4 Hz, 1H), 7.23 (br s, 1H), 6.35 |
| | | | (dd, J = 6.1, 9.4 Hz, 1H), 4.48 (br d, J = 6.6 Hz, 2H), 4.34- |
| | | | 4.15 (m, 1H), 3.88-3.72 (m, 3H), 3.13-3.02 (m, 2H), 2.72- |
| | | | 2.58 (m, 2H), 2.00 (d, J = 5.3 Hz, 3H), 1.92-1.79 (m, 4H), |
| | | | 1.78-1.66 (m, 1H), 1.66-1.51 (m, 1H), 1.49-1.37 (m, 1H), |
| | | | 1.35-1.22 (m, 1H) |
| 200 | 407 | 408 | δ 11.77-11.62 (m, 1H), 8.45 (d, J = 3.5 Hz, 1H), 8.25 (br s, |
| | | | 1H), 8.03 (br d, J = 7.4 Hz, 1H), 7.86 (br d, J = 7.8 Hz, 1H), |
| | | | 7.65 (t, J = 7.8 Hz, 1H), 7.50 (d, J = 6.8 Hz, 1H), 7.24 (br s, |
| | | | 2H), 6.75-6.62 (m, 2H), 6.57-6.51 (m, 1H), 3.77-3.66 (m, |
| | | | 1H), 2.23 (br s, 1H), 2.05-1.90 (m, 2H), 1.82-1.76 (m, 1H), |
| | | | 1.71 (br d, J = 10.6 Hz, 1H), 1.64-1.54 (m, 1H), 1.40-1.28 |
| | | | (m, 2H), 1.24-1.18 (m, 1H) |
| 201 | 407 | 408 | δ 11.86 (br s, 1H), 8.48-8.38 (m, 1H), 8.08 (s, 1H), 7.86 (br |
| | | | dd, J = 9.4, 2.8 Hz, 2H), 7.77-7.71 (m, 2H), 7.60-7.55 (m, |
| | | | 1H), 7.29-7.19 (m, 2H), 6.68 (br s, 1H), 6.48 (d, J = 9.5 Hz, |
| | | | 1H), 3.81-3.67 (m, 1H), 2.23 (br t, J = 11.6 Hz, 1H), 2.04- |
| | | | 1.98 (m, 1H), 1.94 (br d, J = 10.4 Hz, 1H), 1.83-1.76 (m, 1H), |
| | | | 1.72 (br d, J = 10.9 Hz, 1H), 1.38-1.17 (m, 4H) |
| 202 | 407 | 408 | δ 11.97-11.86 (m, 1H), 8.46 (d, J = 3.5 Hz, 1H), 8.09 (s, 1H), |
| | | | 7.87 (dd, J = 9.5 2.9 Hz, 2H), 7.78-7.71 (m, 2H), 7.61-7.55 |
| | | | (m, 1H), 7.36 (d, J = 7.6 Hz, 1H), 6.48 (d, J = 9.5 Hz, 1H), 4.29 |
| | | | (br d, J = 13.6 Hz, 1H), 4.01-3.89 (m, 1H), 3.87-3.77 (m, |
| | | | 1H), 3.21-3.12 (m, 1H), 2.75 (br t, J = 11.3 Hz, 1H), 2.01 (s, |
| | | | 3H), 1.99-1.86 (m, 2H), 1.50-1.39 (m, 1H), 1.39-1.27 (m, 1H) |
| 203 | 421 | 422 | δ 12.01-11.79 (m, 1H), 8.46-8.41 (m, 1H), 8.11-8.08 (m, |
| | | | 1H), 7.88-7.82 (m, 2H), 7.77-7.72 (m, 3H), 7.60-7.55 (m, |
| | | | 1H), 7.20 (d, J = 7.6 Hz, 1H), 6.48 (d, J = 9.5 Hz, 1H), 3.70- |
| | | | 3.60 (m, 1H), 3.56-3.45 (m, 1H), 2.02-1.93 (m, 2H), 1.85- |
| | | | 1.80 (m, 2H), 1.78 (s, 3H), 1.41-1.31 (m, 2H), 1.30 (br d, J = |
| | | | 5.1 Hz, 2H) |
| 204 | 407 | 408 | δ 11.88 (br s, 1H), 8.42 (d, J = 3.6 Hz, 1H), 8.36 (br s, 1H), |
| | | | 7.92-7.83 (m, 2H), 7.72 (dd, J = 6.9 Hz, 2.0, 1H), 7.55 (t, J = |
| | | | 7.8 Hz, 1H), 7.44 (dd, J = 6.9 Hz, 2.0, 1H), 7.25 (br s, 2H), 6.69 |
| | | | (br s, 1H), 6.34 (t, J = 6.7 Hz, 1H), 3.73 (br s, 1H), 2.29-2.18 |
| | | | (m, 1H), 2.02 (br d, J = 10.9 Hz, 1H), 1.93 (br d, J = 10.0 Hz, |
| | | | 1H), 1.82-1.74 (m, 1H), 1.70 (br d, J = 11.5 Hz, 1H), 1.39- |
| | | | 1.28 (m, 2H), 1.27-1.14 (m, 2H) |
| 205 | 323 | 324 | δ 7.72 (d, J = 7.0 Hz, 1H), 4.41-4.37 (m, 1H), 3.91-3.87 (m, |
| | | | 2H), 3.75-3.74 (m, 4H), 3.72 (s, 4H), 3.25 (ddd, J = 13.9, 11.6, |
| | | | 2.9 Hz, 1H), 2.90-2.83 (m, 1H), 2.11 (s, 3H), 2.05-1.95 (m, |
| | | | 2H), 1.49-1.37 (m, 2H) |
| 206 | 413 | 414 | δ 8.52-8.39 (m, 1H), 8.00 (br s, 1H), 7.87 (br s, 1H), 7.58 (br |
| | | | d, J = 2.5 Hz, 2H), 7.34 (br d, J = 4.8 Hz, 1H), 4.28 (br s, 1H), |
| | | | 4.24 (br d, J = 3.0 Hz, 2H), 4.00 (br s, 2H), 3.94 (br d, J = 2.9 |
| | | | Hz, 1H), 3.79 (br d, J = 2.5 Hz, 3H), 3.15 (br t, J = 11.4 Hz, |
| | | | 1H), 2.84-2.67 (m, 1H), 2.01 (br d, J = 3.1 Hz, 3H), 1.97- |
| | | | 1.79 (m, 2H), 1.52-1.23 (m, 2H) |
| 207 | 421 | 422 | δ 8.46 (d, J = 3.6 Hz, 1H), 8.03 (br d, J = 7.8 Hz, 1H), 7.98 (s, |
| | | | 1H), 7.71-7.65 (m, 1H), 7.59 (dd, J = 0.9, 7.9 Hz, 1H), 7.53 (s, |
| | | | 1H), 7.43 (dd, J = 2.5, 9.4 Hz, 1H), 7.29 (br d, J = 8.0 Hz, 1H), |
| | | | 7.22 (br s, 1H), 6.68 (br s, 1H), 6.47 (d, J = 9.4 Hz, 1H), 3.77- |
| | | | 3.66 (m, 1H), 2.26-2.16 (m, 1H), 2.07 (s, 3H), 2.02-1.89 (m, |
| | | | 2H), 1.82-1.74 (m, 1H), 1.70 (br d, J = 11.9 Hz, 1H), 1.37- |
| | | | 1.17 (m, 4H) |
| 208 | 475 | 476 | δ 8.46 (d, J = 3.6 Hz, 1H), 8.38-8.35 (m, 1H), 8.08 (br d, J = |
| | | | 7.4 Hz, 1H), 8.03 (s, 1H), 7.82-7.78 (m, 1H), 7.74-7.65 (m, |
| | | | 2H), 7.31-7.26 (m, 1H), 7.21 (br s, 1H), 6.70-6.66 (m, 2H), |
| | | | 3.77-3.67 (m, 1H), 2.26-2.16 (m, 1H), 2.01-1.88 (m, 2H), |
| | | | 1.81-1.74 (m, 1H), 1.73-1.65 (m, 1H), 1.28 (br s, 2H), 1.26- |
| | | | 1.16 (m, 2H) |
| 209 | 413 | 414 | δ 8.40 (d, J = 3.8 Hz, 1H), 7.51 (br s, 1H), 7.32 (br t, J = 6.8 |
| | | | Hz, 3H), 7.12-7.07 (m, 1H), 4.86 (br d, J = 4.3 Hz, 1H), 4.29 |
| | | | (br d, J = 13.8 Hz, 1H), 3.96-3.87 (m, 1H), 3.82 (br d, J = 14.3 |
| | | | Hz, 1H), 3.66-3.58 (m, 2H), 3.52 (br d, J = 12.1 Hz, 1H), 3.14 |
| | | | (br t, J = 11.6 Hz, 1H), 2.75-2.67 (m, 2H), 2.59-2.53 (m, 1H), |
| | | | 2.00 (s, 3H), 1.96 (br d, J = 3.5 Hz, 1H), 1.93-1.87 (m, 2H), |
| | | | 1.77 (td, J = 13.2, 3.5 Hz, 1H), 1.59-1.49 (m, 1H), 1.47 (br d, J = |
| | | | 4.0 Hz, 1H), 1.34-1.26 (m, 2H) |
| 210 | 413 | 414 | δ 8.40 (d, J = 3.8 Hz, 1H), 7.51 (br s, 1H), 7.37-7.26 (m, 3H), |
| | | | 7.13-7.06 (m, 1H), 4.85 (d, J = 4.5 Hz, 1H), 4.35-4.25 (m, |
| | | | 1H), 3.96-3.78 (m, 2H), 3.66-3.49 (m, 3H), 3.19-3.10 (m, |
| | | | 1H), 2.77-2.68 (m, 2H), 2.57 (br d, J = 2.3 Hz, 1H), 2.00 (s, |
| | | | 3H), 1.98-1.86 (m, 3H), 1.80-1.72 (m, 1H), 1.54 (br d, J = |
| | | | 11.8 Hz, 1H), 1.43 (br dd, J = 11.3, 3.1Hz, 1H), 1.36-1.24 (m, |
| | | | 2H) |
| 211 | 413 | 414 | δ 8.40 (d, J = 3.6 Hz, 1H), 7.54 (br s, 1H), 7.36-7.30 (m, 2H), |
| | | | 7.27 (d, J = 7.5 Hz, 1H), 7.12 (br d, J = 6.5 Hz, 1H), 4.69 (d, J = |
| | | | 4.1 Hz, 1H), 4.29 (br d, J = 13.3 Hz, 1H), 3.98-3.89 (m, 1H), |
| | | | 3.82 (br d, J = 13.4 Hz, 1H), 3.70-3.62 (m, 1H), 3.61-3.54 |
| | | | (m, 2H), 3.15 (br t, J = 11.7 Hz, 1H), 2.96-2.88 (m, 2H), 2.73 |
| | | | (br t, J = 11.3 Hz, 1H), 2.01 (s, 3H), 1.99-1.87 (m, 2H), 1.86- |
| | | | 1.79 (m, 2H), 1.55-1.41 (m, 3H), 1.37-1.28 (m, 1H) |
| 212 | 427 | 428 | δ 8.47 (d, J = 3.5 Hz, 1H), 8.12 (br d, J = 8.1 Hz, 1H), 8.06 (br |
| | | | d, J = 7.5 Hz, 1H), 7.71-7.66 (m, 1H), 7.65-7.59 (m, 1H), |
| | | | 7.37 (br d, J = 7.0 Hz, 1H), 4.36-4.23 (m, 2H), 3.99-3.90 (m, |
| | | | 1H), 3.82 (br d, J = 13.8 Hz, 1H), 3.63-3.53 (m, 3H), 3.45- |
| | | | 3.38 (m, 1H), 3.26-3.11 (m, 2H), 2.76 (br t, J = 12.2 Hz, 1H), |
| | | | 2.01 (s, 3H), 1.99-1.78 (m, 4H), 1.50-1.41 (m, 1H), 1.37- |
| | | | 1.27 (m, 1H) |
| 213 | 427 | 428 | δ 8.47 (d, J = 3.7 Hz, 1H), 8.12 (br d, J = 8.1 Hz, 1H), 8.06 (br |
| | | | d, J = 7.3 Hz, 1H), 7.71-7.67 (m, 1H), 7.65-7.59 (m, 1H), |
| | | | 7.37 (br d, J = 7.5 Hz, 1H), 5.07-4.96 (m, 1H), 4.37-4.23 (m, |
| | | | 2H), 4.00-3.91 (m, 1H), 3.81 (br d, J = 14.1 Hz, 1H), 3.62- |
| | | | 3.54 (m, 2H), 3.47-3.37 (m, 1H), 3.27-3.12 (m, 2H), 2.76 (br |
| | | | t, J = 11.9 Hz, 1H), 2.01 (s, 3H), 1.99-1.82 (m, 4H), 1.50- |
| | | | 1.39 (m, 1H), 1.39-1.28 (m, 1H) |
| 214 | 413 | 414 | δ 8.48 (d, J = 3.6 Hz, 1H), 8.24 (s, 1H), 8.10 (br d, J = 7.8 Hz, |
| | | | 1H), 7.79 (d, J = 7.8 Hz, 1H), 7.67-7.60 (m, 1H), 7.39 (br d, J = |
| | | | 6.9 Hz, 1H), 4.56-4.46 (m, 2H), 4.33-4.24 (m, 2H), 4.09 |
| | | | (br d, J = 5.5 Hz, 1H), 3.96 (br s, 1H), 3.82 (br d, J = 11.3 Hz, |
| | | | 2H), 3.16 (br t, J = 11.5 Hz, 1H), 2.76 (br t, J = 11.5 Hz, 1H), |
| | | | 2.01 (s, 3H), 1.99-1.86 (m, 2H), 1.52-1.26 (m, 2H) |
| 215 | 434 | 435 | δ 8.14 (s, 1H), 7.81 (d, J = 7.0 Hz, 1H), 6.58 (br s, 1H), 5.35- |
| | | | 4.59 (m, 1H), 4.42-4.16 (m, 4H), 3.80-3.72 (m, 2H), 3.58- |
| | | | 3.52 (m, 2H), 3.38-3.29 (m, 3H), 3.12-3.04 (m, 1H), 3.01- |
| | | | 2.88 (m, 2H), 2.75-2.53 (m, 2H), 1.98 (s, 3H), 1.91-1.75 (m, |
| | | | 4H), 1.73-1.45 (m, 3H), 1.41-1.17 (m, 2H) |
| 216 | 434 | 435 | δ 7.82 (d, J = 7.0 Hz, 1H), 6.63 (br d, J = 7.0 Hz, 1H), 4.97 (br |
| | | | s, 1H), 4.44-4.35 (m, 1H), 4.34-4.27 (m, 2H), 4.25 (br s, 2H), |
| | | | 3.83-3.71 (m, 2H), 3.62-3.51 (m, 2H), 3.27 (br d, J = 3.4 Hz, |
| | | | 1H), 3.13-3.03 (m, 1H), 3.00-2.91 (m, 2H), 2.70-2.63 (m, |
| | | | 1H), 1.99 (d, J = 1.9 Hz, 3H), 1.90-1.75 (m, 5H), 1.74-1.45 |
| | | | (m, 4H), 1.42-1.18 (m, 2H) |
| 217 | 471 | 472 | δ 8.48 (d, J = 3.5 Hz, 1H), 8.04 (br d, J = 6.9 Hz, 1H), 7.96 (s, |
| | | | 1H), 7.66-7.59 (m, 1H), 7.58-7.54 (m, 1H), 7.39 (br d, J = |
| | | | 7.7 Hz, 1H), 4.32-4.23 (m, 2H), 4.01-3.90 (m, 1H), 3.86- |
| | | | 3.77 (m, 1H), 3.42-3.37 (m, 4H), 3.22 (s, 2H), 3.18-3.08 (m, |
| | | | 2H), 2.82-2.72 (m, 1H), 2.02 (s, 3H), 1.99-1.85 (m, 2H), 1.66- |
| | | | 1.52 (m, 2H), 1.51-1.41 (m, 2H), 1.34 (br d, J = 7.8 Hz, 2H) |
| 218 | 470 | 471 | δ 8.46 (d, J = 3.5 Hz, 1H), 8.06-8.00 (m, 1H), 7.96 (s, 1H), |
| | | | 7.63-7.59 (m, 1H), 7.57-7.53 (m, 1H), 7.36 (br d, J = 7.5 Hz, |
| | | | 1H), 6.53 (br s, 1H), 4.27 (br d, J = 13.1 Hz, 2H), 4.19 (br dd, J = |
| | | | 3.5, 2.1 Hz, 2H), 3.95 (br d, J = 6.6 Hz, 2H), 3.81 (br d, J = |
| | | | 13.5 Hz, 1H), 3.16 (br t, J = 11.3 Hz, 2H), 2.97 (br d, J = 4.1 |
| | | | Hz, 2H), 2.76 (br t, J = 11.8 Hz, 1H), 2.01 (s, 3H), 1.97-1.84 |
| | | | (m, 2H), 1.56-1.45 (m, 3H), 1.43 (br s, 1H), 1.41-1.34 (m, |
| | | | 2H), 1.34-1.27 (m, 1H) |
| 219 | 344 | 345 | δ 8.46 (d, J = 4.2 Hz, 1H), 8.25-8.23 (m, 1H), 7.73 (s, 1H), |
| | | | 7.44 (br d, J = 6.1 Hz, 1H), 4.27 (br d, J = 13.2 Hz, 1H), 3.94- |
| | | | 3.92 (m, 1H), 3.80 (br d, J = 13.7 Hz, 1H), 3.18 (br d, J = 9.3 |
| | | | Hz, 1H), 2.75 (br s, 1H), 2.00 (s, 3H), 1.92-1.79 (m, 3H), 1.46- |
| | | | 1.26 (m, 2H), 0.92-0.87 (m, 2H), 0.61 (br d, J = 3.5 Hz, 2H) |
| 220 | 413 | 414 | δ 8.46 (d, J = 3.6 Hz, 1H), 7.97 (s, 1H), 7.85 (br d, J = 5.8 Hz, |
| | | | 1H), 7.60-7.51 (m, 2H), 7.35 (br d, J = 7.7 Hz, 1H), 4.37 (t, J = |
| | | | 5.3 Hz, 2H), 4.28 (br d, J = 12.8 Hz, 1H), 4.03-3.88 (m, 1H), |
| | | | 3.82 (br d, J = 13.4 Hz, 1H), 3.72 (t, J = 6.0 Hz, 2H), 3.21- |
| | | | 3.11 (m, 1H), 2.76 (br t, J = 11.2 Hz, 1H), 2.22-2.07 (m, 2H), |
| | | | 2.01 (s, 3H), 1.92 (br dd, J = 14.6, 18.7 Hz, 2H), 1.49-1.25 (m, |
| | | | 2H) |
| 221 | 411 | 412 | δ 8.44 (d, J = 3.8 Hz, 1H), 7.94-7.78 (m, 2H), 7.64-7.50 (m, |
| | | | 1H), 7.49-7.39 (m, 2H), 4.27 (br d, J = 13.1 Hz, 1H), 3.93 (br |
| | | | t, J = 9.9 Hz, 1H), 3.75-3.85 (m, 1H), 3.64 (t, J = 5.4 Hz, 2H), |
| | | | 3.25-3.05 (m, 1H), 2.74 (br t, J = 11.4 Hz, 1H), 2.41 (br t, J = |
| | | | 6.3 Hz, 2H), 2.00 (s, 3H), 1.94-1.77 (m, 6H), 1.51-1.18 (m, |
| | | | 2H) |
| 222 | 397 | 398 | δ 8.45 (d, J = 3.7 Hz, 1H), 8.37 (br s, 1H), 7.83-7.66 (m, 2H), |
| | | | 7.60-7.50 (m, 1H), 7.33 (br d, J = 7.6 Hz, 1H), 4.29 (br d, J = |
| | | | 12.9 Hz, 1H), 4.03-3.74 (m, 4H), 3.16 (br t, J = 11.8 Hz, 1H), |
| | | | 2.75 (br t, J = 11.9 Hz, 1H), 2.56-2.52 (m, 2H), 2.10 (quin, J = |
| | | | 7.5 Hz, 2H), 2.01 (s, 3H), 1.98-1.83 (m, 2H), 1.50-1.25 (m, |
| | | | 2H) |
| 223 | 393 | 394 | δ 11.93-11.83 (m, 1H), 8.44-8.41 (m, 1H), 8.36-8.31 (m, |
| | | | 1H), 7.90 (br d, J = 8.1 Hz, 1H), 7.86 (br d, J = 7.8 Hz, 1H), |
| | | | 7.71 (dd, J = 7.0, 1.8 Hz, 1H), 7.59-7.52 (m, 1H), 7.46-7.41 |
| | | | (m, 1H), 7.36 (br d, J = 7.3 Hz, 1H), 7.34-7.30 (m, 1H), 6.83- |
| | | | 6.73 (m, 1H), 6.33 (t, J = 6.7 Hz, 1H), 4.24-4.17 (m, 1H), 2.69- |
| | | | 2.65 (m, 1H), 2.21-2.11 (m, 1H), 1.94-1.86 (m, 1H), 1.84- |
| | | | 1.77 (m, 2H), 1.70-1.61 (m, 2H) |
| 224 | 414 | 415 | δ 11.61-11.24 (m, 1H), 7.83 (d, J = 7.0 Hz, 1H), 7.31 (dd, J = |
| | | | 2.9, 6.6 Hz, 1H), 6.63 (br d, J = 6.4 Hz, 1H), 6.26-6.10 (m, |
| | | | 2H), 4.44-4.20 (m, 3H), 3.76 (br d, J = 10.8 Hz, 2H), 3.11- |
| | | | 2.91 (m, 3H), 2.70-2.54 (m, 2H), 1.98 (d, J = 2.3 Hz, 3H), |
| | | | 1.93-1.62 (m, 5H), 1.60-1.48 (m, 1H), 1.40-1.17 (m, 2H) |
| 225 | 427 | 428 | δ 8.43 (d, J = 3.6 Hz, 1H), 7.95 (s, 1H), 7.83 (br d, J = 6.9 Hz, |
| | | | 1H), 7.74 (br d, J = 7.8 Hz, 1H), 7.60-7.48 (m, 2H), 7.19 (br d, |
| | | | J = 7.8 Hz, 1H), 4.37 (t, J = 5.3 Hz, 2H), 3.71 (t, J = 6.0 Hz, |
| | | | 2H), 3.66-3.57 (m, 1H), 3.54-3.43 (m, 1H), 2.19-2.06 (m, |
| | | | 2H), 2.01-1.90 (m, 2H), 1.81 (br d, J = 12.1 Hz, 2H), 1.77 (s, |
| | | | 3H), 1.40-1.16 (m, 4H) |
| 226 | 399 | 340 | δ 8.45 (d, J = 3.6 Hz, 1H), 8.36-8.24 (m, 1H), 7.77-7.64 (m, |
| | | | 2H), 7.62-7.50 (m, 1H), 7.33 (br d, J = 7.5 Hz, 1H), 4.47 (t, J = |
| | | | 7.9 Hz, 2H), 4.28 (br d, J = 13.1 Hz, 1H), 4.12 (t, J = 7.9 Hz, |
| | | | 2H), 4.01-3.89 (m, 1H), 3.81 (br d, J = 14.1 Hz, 1H), 3.21- |
| | | | 3.09 (m, 1H), 2.74 (br t, J = 11.7 Hz, 1H), 2.00 (s, 3H), 1.91 (br |
| | | | d, J = 18.3 Hz, 2H), 1.48-1.25 (m, 2H) |
| 227 | 413 | 414 | δ 8.42 (d, J = 3.5 Hz, 1H), 7.96 (s, 1H), 7.83 (br d, J = 6.3 Hz, |
| | | | 1H), 7.60-7.48 (m, 2H), 7.31-7.15 (m, 2H), 6.68 (br s, 1H), |
| | | | 4.37 (t, J = 5.3 Hz, 2H), 3.71 (br t, J = 6.0 Hz, 3H), 2.22 (br t, J = |
| | | | 11.9 Hz, 1H), 2.12 (quin, J = 5.7 Hz, 2H), 2.05-1.86 (m, |
| | | | 2H), 1.82-1.64 (m, 2H), 1.40-1.13 (m, 4H) |
| 228 | 413 | 414 | δ 8.45 (d, J = 3.8 Hz, 1H), 8.41-8.37 (m, 1H), 7.84-7.71 (m, |
| | | | 2H), 7.59-7.51 (m, 1H), 7.33 (d, J = 7.6 Hz, 1H), 5.79 (d, J = |
| | | | 5.9 Hz, 1H), 4.42-4.22 (m, 2H), 4.00-3.89 (m, 1H), 3.86- |
| | | | 3.71 (m, 3H), 3.21-3.11 (m, 1H), 2.80-2.65 (m, 1H), 2.46- |
| | | | 2.31 (m, 1H), 2.01 (s, 3H), 1.97-1.82 (m, 3H), 1.50-1.40 (m, |
| | | | 1H), 1.38-1.28 (m, 1H) |
| 229 | 407 | 408 | δ 2.04-11.78 (m, 1H), 8.44 (d, J = 3.6 Hz, 1H), 8.09 (s, 1H), |
| | | | 7.86 (dd, J = 9.4, 2.7 Hz, 2H), 7.80-7.69 (m, 2H), 7.66-7.53 |
| | | | (m, 1H), 7.26-7.11 (m, 2H), 6.74-6.63 (m, 1H), 6.47 (d, J = |
| | | | 9.4 Hz, 1H), 3.71-3.60 (m, 1H), 2.12-1.95 (m, 3H), 1.80 (br |
| | | | d, J = 11.9 Hz, 2H), 1.49-1.37 (m, 2H), 1.34-1.22 (m, 2H) |
| 230 | 413 | 414 | δ 8.44 (d, J = 3.6 Hz, 1H), 8.38 (br s, 1H), 7.84-7.66 (m, 2H), |
| | | | 7.53 (t, J = 8.0 Hz, 1H), 7.38-7.25 (m, 1H), 5.37 (d, J = 3.9 |
| | | | Hz, 1H), 4.42 (br d, J = 3.3 Hz, 1H), 4.34-4.23 (m, 1H), 4.11 |
| | | | (dd, J = 5.1, 10.4 Hz, 1H), 4.01-3.88 (m, 1H), 3.82 (br d, J = |
| | | | 13.5 Hz, 1H), 3.64 (br d, J = 10.4 Hz, 1H), 3.21-3.11 (m, 1H), |
| | | | 2.86 (dd, J = 6.1, 17.0 Hz, 1H), 2.81-2.65 (m, 1H), 2.38-2.29 |
| | | | (m, 1H), 2.01 (s, 3H), 1.99-1.86 (m, 2H), 1.51-1.25 (m, 2H) |
| 231 | 399 | 400 | δ 8.74 (d, J = 4.9 Hz, 1H), 8.52 (d, J = 3.3 Hz, 1H), 8.15 (s, |
| | | | 1H), 7.78 (br d, J = 8.1 Hz, 1H), 7.37 (br d, J = 6.7 Hz, 1H), |
| | | | 5.90 (br s, 1H), 3.72-3.61 (m, 1H), 3.56-3.45 (m, 1H), 2.63- |
| | | | 2.56 (m, 2H), 2.33-2.21 (m, 2H), 2.03-1.92 (m, 3H), 1.90- |
| | | | 1.81 (m, 3H), 1.79 (s, 3H), 1.43-1.19 (m, 5H) |
| 232 | 385 | 386 | δ 8.75 (d, J = 5.1 Hz, 1H), 8.55 (d, J = 3.4 Hz, 1H), 8.15 (s, |
| | | | 1H), 7.77 (br d, J = 2.6 Hz, 1H), 7.51 (br d, J = 7.3 Hz, 1H), |
| | | | 6.25-5.91 (m, 1H), 4.30 (br d, J = 13.2 Hz, 1H), 4.01-3.91 |
| | | | (m, 1H), 3.83 (br d, J = 13.7 Hz, 1H), 3.17 (br t, J = 11.7 Hz, |
| | | | 1H), 2.76 (br t, J = 11.8 Hz, 1H), 2.66-2.56 (m, 2H), 2.32- |
| | | | 2.22 (m, 2H), 2.03 (s, 3H), 2.00-1.85 (m, 4H), 1.52-1.42 (m, |
| | | | 1H), 1.40-1.28 (m, 1H) |
| 233 | 413 | 414 | δ 8.43 (d, J = 3.5 Hz, 1H), 8.39-8.16 (m, 1H), 7.75 (br d, J = |
| | | | 7.8 Hz, 1H), 7.72-7.65 (m, 2H), 7.59-7.53 (m, 1H), 7.18 (br |
| | | | d, J = 7.7 Hz, 1H), 4.48 (t, J = 7.9 Hz, 2H), 4.16-4.09 (m, 2H), |
| | | | 3.65 (br dd, J = 2.8, 6.5 Hz, 1H), 3.53-3.46 (m, 1H), 2.03- |
| | | | 1.94 (m, 2H), 1.78 (s, 4H), 1.40-1.19 (m, 5H) |
| 234 | 489 | 490 | δ 8.47 (d, J = 3.6 Hz, 1H), 8.38-8.35 (m, 1H), 8.10-8.06 (m, |
| | | | 1H), 8.02 (s, 1H), 7.83-7.78 (m, 1H), 7.75-7.65 (m, 3H), 7.24- |
| | | | 7.20 (m, 1H), 6.69 (d, J = 9.6 Hz, 1H), 3.69-3.60 (m, 1H), |
| | | | 3.53-3.44 (m, 1H), 1.96 (br d, J = 10.1 Hz, 2H), 1.83-1.77 |
| | | | (m, 5H), 1.38-1.31 (m, 2H), 1.28-1.18 (m, 2H) |
| 235 | 435 | 436 | δ 8.46 (d, J = 3.6 Hz, 1H), 8.03 (br d, J = 7.8 Hz, 1H), 7.96 (s, |
| | | | 1H), 7.75 (br d, J = 7.6 Hz, 1H), 7.71-7.65 (m, 1H), 7.61- |
| | | | 7.58 (m, 1H), 7.53 (s, 1H), 7.43 (dd, J = 2.4, 9.3 Hz, 1H), 7.24 |
| | | | (br d, J = 7.9 Hz, 1H), 6.47 (d, J = 9.3 Hz, 1H), 3.69-3.59 (m, |
| | | | 1H), 3.54-3.44 (m, 1H), 2.07 (s, 3H), 1.96 (br d, J = 9.8 Hz, |
| | | | 2H), 1.82 (br s, 2H), 1.77 (s, 3H), 1.36-1.21 (m, 4H) |
| 236 | 413 | 414 | δ 8.43 (d, J = 3.5 Hz, 1H), 8.16 (s, 0.15H), 7.94 (s, 1H), 7.84 |
| | | | (br d, J = 6.6 Hz, 1H), 7.64-7.47 (m, 2H), 7.28-7.09 (m, 2H), |
| | | | 6.68 (br s, 1H), 4.36 (t, J = 5.3 Hz, 2H), 3.76-3.57 (m, 3H), |
| | | | 2.22-1.90 (m, 5H), 1.79 (br d, J = 11.8 Hz, 2H), 1.51-1.35 |
| | | | (m, 2H), 1.32-1.18 (m, 2H) |
| 237 | 413 | 414 | δ 8.51-8.31 (m, 2H), 7.81-7.66 (m, 2H), 7.62-7.47 (m, 1H), |
| | | | 7.32 (br d, J = 7.6 Hz, 1H), 5.39 (br d, J = 3.3 Hz, 1H), 4.42 (br |
| | | | s, 1H), 4.28 (br d, J = 13.3 Hz, 1H), 4.11 (dd, J = 5.1, 10.4 Hz, |
| | | | 1H), 4.00-3.88 (m, 1H), 3.81 (br d, J = 13.6 Hz, 1H), 3.64 (br |
| | | | d, J = 10.3 Hz, 1H), 3.16 (br t, J = 11.7 Hz, 1H), 2.86 (dd, J = |
| | | | 6.1, 17.1 Hz, 1H), 2.75 (br t, J = 11.7 Hz, 1H), 2.33 (br d, J = |
| | | | 17.8 Hz, 1H), 2.01 (s, 3H), 1.98-1.82 (m, 2H), 1.56-1.22 (m, |
| | | | 2H) |
| 238 | 421 | 422 | δ 8.46 (d, J = 3.6 Hz, 1H), 8.10-8.02 (m, 1H), 7.98 (s, 1H), |
| | | | 7.79-7.65 (m, 3H), 7.63-7.51 (m, 2H), 7.23 (d, J = 7.9 Hz, |
| | | | 1H), 6.52 (d, J = 9.0 Hz, 1H), 6.36 (dt, J = 1.3, 6.7 Hz, 1H), |
| | | | 3.72-3.58 (m, 1H), 3.49 (dt, J = 3.7, 7.3 Hz, 1H), 1.96 (br d, J = |
| | | | 11.0 Hz, 2H), 1.82 (s, 2H), 1.74 (s, 3H), 1.42-1.14 (m, 4H) |
| 239 | 380 | 381 | δ 12.66 (s, 1H), 8.58 (s, 1H), 8.48 (d, J = 3.5 Hz, 1H), 8.07 (d, J = |
| | | | 7.9 Hz, 1H), 7.91 (br d, J = 7.0 Hz, 1H), 7.62 (t, J = 7.9 Hz, |
| | | | 1H), 7.38 (br d, J = 7.9 Hz, 1H), 7.29 (s, 1H), 7.08-7.06 (m, |
| | | | 1H), 4.30-4.27 (m, 1H), 4.01-3.94 (m, 1H), 3.82 (br d, J = |
| | | | 13.9 Hz, 1H), 3.20-3.14 (m, 1H), 2.80-2.73 (m, 1H), 2.02 (s, |
| | | | 3H), 1.98-1.89 (m, 2H), 1.51-1.32 (m, 2H) |
| 240 | 407 | 408 | δ 8.46 (d, J = 3.7 Hz, 1H), 8.05 (br d, J = 7.6 Hz, 1H), 7.96 (br |
| | | | s, 1H), 7.75-7.67 (m, 2H), 7.62-7.57 (m, 1H), 7.54 (ddd, J = |
| | | | 9.0, 6.7, 2.1 Hz, 1H), 7.24 (br d, J = 7.8 Hz, 1H), 7.21 (br s, |
| | | | 1H), 6.69 (br s, 1H), 6.51 (d, J = 9.2 Hz, 1H), 6.35 (dt, J = 1.3, |
| | | | 6.7 Hz, 1H), 3.70-3.55 (m, 1H), 2.09-2.02 (m, 1H), 2.01- |
| | | | 1.93 (m, 2H), 1.77 (br d, J = 11.6 Hz, 2H), 1.47-1.34 (m, 2H), |
| | | | 1.31-1.23 (m, 2H) |
| 241 | 387 | 388 | δ 8.67 (d, J = 5.0 Hz, 1H), 8.51 (d, J = 3.4 Hz, 1H), 8.23 (s, |
| | | | 1H), 7.78-7.68 (m, 2H), 7.33 (br d, J = 7.4 Hz, 1H), 5.34 (s, |
| | | | 1H), 3.72-3.60 (m, 1H), 3.56-3.45 (m, 1H), 1.97 (br d, J = |
| | | | 9.6 Hz, 2H), 1.83 (br d, J = 11.9 Hz, 2H), 1.78 (s, 3H), 1.48 (s, |
| | | | 6H), 1.40-1.20 (m, 4H) |
| 242 | 373 | 374 | δ 8.68 (d, J = 5.1 Hz, 1H), 8.52 (d, J = 3.3 Hz, 1H), 8.25 (br s, |
| | | | 1H), 7.76 (br s, 1H), 7.35 (br d, J = 6.1 Hz, 1H), 7.21 (br s, 1H), |
| | | | 6.69 (br s, 1H), 3.70-3.63 (m, 1H), 2.12-1.95 (m, 3H), 1.80 |
| | | | (br d, J = 11.5 Hz, 2H), 1.49 (s, 6H), 1.47-1.38 (m, 2H), 1.35- |
| | | | 1.22 (m, 2H) |
| 243 | 385 | 386 | δ 8.73 (d, J = 5.1 Hz, 1H), 8.50 (d, J = 3.6 Hz, 1H), 8.13 (s, |
| | | | 1H), 7.74 (br s, 1H), 7.33 (br d, J = 7.3 Hz, 1H), 7.20 (br s, 1H), |
| | | | 6.68 (br s, 1H), 5.87 (s, 1H), 3.71-3.58 (m, 1H), 2.61-2.54 |
| | | | (m, 2H), 2.30-2.21 (m, 2H), 2.11-2.04 (m, 1H), 2.03-1.96 |
| | | | (m, 2H), 1.95-1.89 (m, 1H), 1.88-1.82 (m, 1H), 1.79 (br d, J = |
| | | | 13.5 Hz, 2H), 1.50-1.36 (m, 2H), 1.29 (br s, 2H) |
| 244 | 385 | 386 | δ 8.72 (d, J = 5.0 Hz, 1H), 8.50 (d, J = 3.3 Hz, 1H), 8.12 (s, |
| | | | 1H), 7.73 (br d, J = 2.8 Hz, 1H), 7.41 (br d, J = 7.9 Hz, 1H), |
| | | | 7.21 (br s, 1H), 6.69 (br s, 1H), 5.89 (s, 1H), 3.77-3.68 (m, |
| | | | 1H), 2.61-2.54 (m, 2H), 2.29-2.20 (m, 3H), 2.02-1.85 (m, |
| | | | 4H), 1.81-1.67 (m, 2H), 1.37-1.17 (m, 4H) |
| 245 | 399 | 400 | δ 8.43 (d, J = 3.6 Hz, 1H), 8.38-8.15 (m, 1H), 7.68 (br d, J = |
| | | | 9.1 Hz, 2H), 7.60-7.52 (m, 1H), 7.24-7.14 (m, 2H), 6.69 (br |
| | | | s, 1H), 4.48 (dd, J = 7.1, 8.9 Hz, 2H), 4.16-4.09 (m, 2H), 3.70- |
| | | | 3.60 (m, 1H), 2.08-1.96 (m, 3H), 1.79 (br d, J = 11.5 Hz, |
| | | | 2H), 1.43 (q, J = 11.9 Hz, 2H), 1.34-1.20 (m, 2H) |
| 246 | 475 | 476 | δ 8.47 (d, J = 3.5 Hz, 1H), 8.36 (s, 1H), 8.11-8.07 (m, 1H), |
| | | | 8.02 (s, 1H), 7.82-7.78 (m, 1H), 7.75-7.65 (m, 2H), 7.25- |
| | | | 7.18 (m, 2H), 6.71-6.66 (m, 2H), 3.70-3.60 (m, 1H), 2.09- |
| | | | 1.96 (m, 3H), 1.81-1.76 (m, 2H), 1.47-1.36 (m, 2H), 1.31- |
| | | | 1.21 (m, 2H) |
| 247 | 421 | 422 | δ 8.46 (d, J = 3.5 Hz, 1H), 8.04 (br d, J = 7.6 Hz, 1H), 7.97 (s, |
| | | | 1H), 7.71-7.65 (m, 1H), 7.59 (td, J = 8.0, 1.0 Hz, 1H), 7.53 (s, |
| | | | 1H), 7.42 (dd, J = 9.4, 2.5 Hz, 1H), 7.27-7.18 (m, 2H), 6.70 |
| | | | (br s, 1H), 6.47 (d, J = 9.3 Hz, 1H), 3.69-3.60 (m, 1H), 2.07 (s, |
| | | | 3H), 2.05-1.94 (m, 3H), 1.78 (br d, J = 11.3 Hz, 2H), 1.47- |
| | | | 1.36 (m, 2H), 1.31-1.20 (m, 2H) |
| 248 | 411 | 412 | δ 8.47-8.24 (m, 2H), 7.83-7.63 (m, 3H), 7.59-7.47 (m, 1H), |
| | | | 7.15 (d, J = 7.6 Hz, 1H), 3.88 (t, J = 7.0 Hz, 2H), 3.73-3.56 |
| | | | (m, 1H), 3.55-3.41 (m, 1H), 2.57-2.53 (m, 2H), 2.09 (quin, J = |
| | | | 7.5 Hz, 2H), 1.97 (br d, J = 11.0 Hz, 2H), 1.81 (br d, J = 11.8 |
| | | | Hz, 2H), 1.78 (s, 3H), 1.42-1.14 (m, 4H) |
| 249 | 397 | 398 | δ 8.41 (d, J = 3.6 Hz, 2H), 7.86-7.64 (m, 2H), 7.59-7.47 (m, |
| | | | 1H), 7.24-7.10 (m, 2H), 6.68 (br s, 1H), 3.88 (t, J = 7.0 Hz, |
| | | | 2H), 3.70-3.58 (m, 1H), 2.57-2.52 (m, 2H), 2.14-1.94 (m, |
| | | | 5H), 1.79 (br d, J = 11.4 Hz, 2H), 1.43 (br d, J = 13.1 Hz, 2H), |
| | | | 1.27 (br d, J = 12.5 Hz, 2H) |
| 250 | 427 | 428 | δ 8.37 (d, J = 3.8 Hz, 1H), 7.78-7.70 (m, 1H), 7.57-7.49 (m, |
| | | | 1H), 7.37-7.25 (m, 2H), 7.15-7.07 (m, 2H), 4.73-4.66 (m, |
| | | | 1H), 3.68-3.60 (m, 2H), 3.60-3.54 (m, 2H), 3.52-3.44 (m, |
| | | | 1H), 2.95-2.86 (m, 2H), 1.97 (br dd, J = 6.5, 1.9 Hz, 2H), 1.81 |
| | | | (br d, J = 4.5 Hz, 4H), 1.77 (s, 3H), 1.48 (br d, J = 9.6 Hz, 2H), |
| | | | 1.39-1.28 (m, 2H), 1.27-1.16 (m, 2H) |
| 251 | 413 | 414 | δ 8.38 (d, J = 3.8 Hz, 1H), 7.59 (br s, 1H), 7.37 (br d, J = 4.3 |
| | | | Hz, 2H), 7.24-7.06 (m, 3H), 6.70 (br s, 1H), 3.72-3.56 (m, |
| | | | 5H), 2.95 (br t, J = 9.7 Hz, 2H), 2.13-1.96 (m, 3H), 1.82 (br |
| | | | dd, J = 17.2, 13.9 Hz, 4H), 1.57-1.37 (m, 4H), 1.34-1.19 (m, |
| | | | 2H) |
| 252 | 441 | 442 | δ 8.45 (d, J = 3.5 Hz, 1H), 8.11 (br s, 1H), 8.05 (br d, J = 7.5 |
| | | | Hz, 1H), 7.78-7.72 (m, 1H), 7.71-7.66 (m, 1H), 7.65-7.58 |
| | | | (m, 1H), 7.33-7.15 (m, 1H), 4.38-4.21 (m, 1H), 3.71-3.41 |
| | | | (m, 7H), 2.02-1.93 (m, 3H), 1.86-1.77 (m, 6H), 1.37-1.21 |
| | | | (m, 4H) |
| 253 | 414 | 415 | δ 8.87-8.62 (m, 1H), 8.55-8.49 (m, 2H), 7.75 (br d, J = 7.8 |
| | | | Hz, 1H), 7.62 (br s, 1H), 7.33 (br d, J = 7.8 Hz, 1H), 4.51-4.44 |
| | | | (m, 2H), 4.26-4.18 (m, 2H), 3.70-3.61 (m, 1H), 3.53-3.44 |
| | | | (m, 1H), 2.03-1.90 (m, 2H), 1.87-1.73 (m, 5H), 1.39-1.20 |
| | | | (m, 4H) |
| 254 | 407 | 408 | δ 8.40 (s, 1H), 8.26 (d, J = 3.9 Hz, 1H), 8.04 (br d, J = 7.5 Hz, |
| | | | 1H), 7.87-7.75 (m, 2H), 7.56 (t, J = 7.8 Hz, 1H), 7.48 (dd, J = |
| | | | 6.4, 1.9 Hz, 1H), 6.54 (t, J = 6.8 Hz, 1H), 3.87-3.76 (m, 1H), |
| | | | 2.33-2.15 (m, 3H), 1.96 (br d, J = 11.8 Hz, 2H), 1.65 (dq, J = |
| | | | 2.9, 13.0 Hz, 2H), 1.43-1.27 (m, 2H) |
| 255 | 421 | 422 | δ 12.10 (s, 1H), 8.41 (d, J = 3.6 Hz, 1H), 8.31 (s, 1H), 7.94- |
| | | | 7.82 (m, 2H), 7.80-7.65 (m, 2H), 7.54 (t, J = 7.8 Hz, 1H), 7.43 |
| | | | (dd, J = 1.9, 6.4 Hz, 1H), 7.15 (br d, J = 7.8 Hz, 1H), 6.33 (t, J = |
| | | | 6.7 Hz, 1H), 3.73-3.59 (m, 1H), 3.56-3.42 (m, 1H), 2.10- |
| | | | 1.92 (m, 2H), 1.82 (br s, 2H), 1.77 (s, 3H), 1.41-1.15 (m, 4H) |
| 256 | 425 | 426 | δ 8.42 (d, J = 3.6 Hz, 1H), 7.89-7.79 (m, 2H), 7.73 (d, J = 7.8 |
| | | | Hz, 1H), 7.54 (t, J = 7.9 Hz, 1H), 7.44 (dd, J = 0.8, 8.1 Hz, 1H), |
| | | | 7.18 (br d, J = 7.8 Hz, 1H), 3.64 (br t, J = 5.5 Hz, 3H), 3.48 (dt, |
| | | | J = 3.6, 7.4 Hz, 1H), 2.41 (t, J = 6.3 Hz, 2H), 1.95 (br d, J = |
| | | | 10.9 Hz, 2H), 1.91-1.79 (m, 6H), 1.77 (s, 3H), 1.41-1.17 (m, |
| | | | 4H) |
| 257 | 426 | 427 | δ 8.59 (d, J = 5.1 Hz, 1H), 8.50 (d, J = 3.3 Hz, 1H), 8.27 (br s, |
| | | | 1H), 7.75 (d, J = 7.7 Hz, 1H), 7.67 (br d, J = 5.0 Hz, 1H), 7.33 |
| | | | (d, J = 7.8 Hz, 1H), 3.89 (br t, J = 5.5 Hz, 2H), 3.69-3.60 (m, |
| | | | 1H), 3.54-3.45 (m, 1H), 2.00-1.92 (m, 2H), 1.91-1.79 (m, |
| | | | 7H), 1.77 (s, 3H), 1.41-1.15 (m, 5H) |
| 258 | 412 | 413 | δ 8.60 (d, J = 5.1 Hz, 1H), 8.51 (d, J = 3.3 Hz, 1H), 8.27 (br s, |
| | | | 1H), 7.68 (br d, J = 4.4 Hz, 1H), 7.34 (br s, 1H), 7.21 (br s, 1H), |
| | | | 6.69 (br s, 1H), 3.89 (t, J = 5.6 Hz, 2H), 3.64 (br s, 1H), 2.10- |
| | | | 2.02 (m, 1H), 1.98 (br d, J = 10.4 Hz, 2H), 1.93-1.84 (m, 4H), |
| | | | 1.82 (br s, 4H), 1.47-1.37 (m, 2H), 1.32-1.23 (m, 2H) |
| 259 | 427 | 428 | δ 8.48 (d, J = 3.8 Hz, 1H), 8.13 (br d, J = 4.1 Hz, 1H), 8.07 (br |
| | | | d, J = 7.5 Hz, 1H), 7.73-7.68 (m, 1H), 7.66-7.60 (m, 1H), |
| | | | 6.93-6.63 (m, 1H), 4.39-4.21 (m, 1H), 3.74-3.50 (m, 4H), |
| | | | 3.49-3.39 (m, 1H), 3.24 (br d, J = 10.9 Hz, 1H), 2.16-2.05 |
| | | | (m, 1H), 2.03-1.90 (m, 3H), 1.88-1.76 (m, 3H), 1.51-1.38 |
| | | | (m, 2H), 1.35-1.22 (m, 2H) |
| 260 | 411 | 412 | δ 9.10 (s, 1H), 8.53 (d, J = 4.0 Hz, 1H), 8.42-8.35 (m, 1H), |
| | | | 8.03 (dd, J = 6.9, 1.4 Hz, 1H), 7.75 (br d, J = 7.8 Hz, 1H), 7.51 |
| | | | (ddd, J = 9.1, 6.8, 1.9 Hz, 1H), 7.46 (d, J = 7.9 Hz, 1H), 6.57 |
| | | | (d, J = 9.1 Hz, 1H), 6.41 (dt, J = 6.8, 1.1 Hz, 1H), 3.71-3.58 |
| | | | (m, 1H), 3.55-3.42 (m, 1H), 1.95 (br d, J = 10.0 Hz, 2H), 1.81 |
| | | | (br d, J = 11.4 Hz, 2H), 1.76 (s, 3H) 1.39-1.19 (m, 4H) |
| 261 | 412 | 413 | δ 8.97 (br s, 1H), 8.56 (d, J = 5.3 Hz, 1H), 8.53 (d, J = 3.4 Hz, |
| | | | 1H), 7.78 (d, J = 7.8 Hz, 1H), 7.62 (br s, 1H), 7.34 (br d, J = 7.8 |
| | | | Hz, 1H), 4.05 (t, J = 7.1 Hz, 2H), 3.76-3.61 (m, 1H), 3.58- |
| | | | 3.44 (m, 1H), 2.63 (t, J = 8.1 Hz, 2H), 2.09 (quin, J = 7.6 Hz, |
| | | | 2H), 2.04-1.93 (m, 2H), 1.83 (br d, J = 11.0 Hz, 2H), 1.79 (s, |
| | | | 3H), 1.43-1.19 (m, 4H) |
| 262 | 398 | 399 | δ 8.93 (br d, J = 2.0 Hz, 1H), 8.54 (d, J = 5.3 Hz, 1H), 8.50 (d, |
| | | | J = 3.4 Hz, 1H), 7.61 (br d, J = 1.6 Hz, 1H), 7.31 (br d, J = 7.6 |
| | | | Hz, 1H), 7.20 (br s, 1H), 6.68 (br s, 1H), 4.03 (t, J = 7.1 Hz, |
| | | | 2H), 3.71-3.57 (m, 1H), 2.61 (t, J = 8.1 Hz, 2H), 2.10-2.05 |
| | | | (m, 3H), 2.03-1.97 (m, 2H), 1.79 (br d, J = 11.5 Hz, 2H), 1.45- |
| | | | 1.32 (m, 2H), 1.29-1.26 (m, 2H) |
| 263 | 400 | 401 | δ 8.89-8.65 (m, 1H), 8.56-8.53 (m, 2H), 7.66-7.63 (m, 1H), |
| | | | 7.36 (br d, J = 7.7 Hz, 1H), 7.24-7.22 (m, 1H), 6.70 (br d, J = |
| | | | 0.9 Hz, 1H), 4.52-4.48 (m, 2H), 4.23 (br t, J = 7.8 Hz, 2H), |
| | | | 3.70-3.63 (m, 1H), 2.07-2.00 (m, 3H), 1.81-1.78 (m, 2H), |
| | | | 1.53-1.37 (m, 2H), 1.30-1.27 (m, 2H) |
| 264 | 411 | 412 | δ 8.42 (d, J = 3.6 Hz, 1H), 7.90-7.79 (m, 2H), 7.54 (t, J = 7.9 |
| | | | Hz, 1H), 7.48-7.41 (m, 1H), 7.20 (br s, 2H), 6.68 (br s, 1H), |
| | | | 3.64 (br t, J = 5.4 Hz, 3H), 2.41 (t, J = 6.3 Hz, 2H), 2.12-1.94 |
| | | | (m, 3H), 1.93-1.83 (m, 4H), 1.79 (br d, J = 11.5 Hz, 2H), 1.51- |
| | | | 1.35 (m, 2H), 1.34-1.18 (m, 2H) |
| 265 | 422 | 423 | δ 8.78 (dd, J = 4.5, 2.2 Hz, 1H), 8.54 (br s, 1H), 8.29 (br s, 1H), |
| | | | 8.01-7.92 (m, 2H), 7.76 (br d, J = 6.3 Hz, 1H), 7.60-7.53 (m, |
| | | | 1H), 7.36 (br d, J = 6.4 Hz, 1H), 6.55 (br d, J = 9.1 Hz, 1H), |
| | | | 6.44-6.37 (m, 1H), 3.70-3.61 (m, 1H), 3.48 (br s, 1H), 1.96 |
| | | | (br d, J = 8.9 Hz, 2H), 1.84-1.76 (m, 5H), 1.39-1.19 (m, 4H) |
| 266 | 428 | 429 | δ 8.59 (d, J = 5.3 Hz, 1H), 8.53 (d, J = 3.3 Hz, 1H), 8.48-8.36 |
| | | | (m, 1H), 7.77 (d, J = 7.8 Hz, 1H), 7.68 (br d, J = 4.4 Hz, 1H), |
| | | | 7.35 (d, J = 7.8 Hz, 1H), 4.39 (t, J = 5.4 Hz, 2H), 3.98 (t, J = 6.2 |
| | | | Hz, 2H), 3.70-3.62 (m, 1H), 3.56-3.47 (m, 1H), 2.17-2.13 |
| | | | (m, 2H), 1.97 (br d, J = 9.2 Hz, 2H), 1.84 (br s, 2H), 1.79 (s, |
| | | | 3H), 1.41-1.32 (m, 2H), 1.31-1.24 (m, 2H) |
| 267 | 427 | 428 | δ 8.43 (d, J = 3.6 Hz, 1H), 7.99 (s, 1H), 7.85 (br d, J = 5.1 Hz, |
| | | | 1H), 7.73 (d, J = 7.6 Hz, 1H), 7.61-7.56 (m, 2H), 7.19 (d, J = |
| | | | 7.9 Hz, 1H), 4.24 (s, 2H), 4.02-3.98 (m, 2H), 3.78 (t, J = 4.9 |
| | | | Hz, 2H), 3.64 (br dd, J = 7.1, 3.4 Hz, 1H), 3.54-3.45 (m, 1H), |
| | | | 1.96 (br d, J = 10.6 Hz, 2H), 1.84-1.75 (m, 5H), 1.39-1.20 |
| | | | (m, 4H) |
| 268 | 413 | 414 | δ 8.43 (d, J = 3.6 Hz, 1H), 7.99 (s, 1H), 7.87 (br d, J = 6.1 Hz, |
| | | | 1H), 7.62-7.54 (m, 2H), 7.20 (br s, 2H), 6.68 (br s, 1H), 4.23 |
| | | | (s, 2H), 4.00 (dd, J = 5.8, 4.2 Hz, 2H), 3.81-3.76 (m, 2H), 3.69- |
| | | | 3.59 (m, 1H), 2.06 (br d, J = 6.0 Hz, 1H), 1.99 (br d, J = 10.4 |
| | | | Hz, 2H), 1.79 (br d, J = 11.4 Hz, 2H), 1.42 (br d, J = 13.6 Hz, |
| | | | 2H), 1.28 (br s, 1H), 1.25 (br s, 1H) |
| 269 | 413 | 414 | δ 8.43 (d, J = 3.6 Hz, 1H), 7.99 (s, 1H), 7.85 (br d, J = 4.8 Hz, |
| | | | 1H), 7.61-7.55 (m, 2H), 7.27 (br d, J = 5.5 Hz, 1H), 7.21 (br s, |
| | | | 1H), 6.68 (br s, 1H), 4.23 (s, 2H), 4.00 (t, J = 5.0 Hz, 2H), 3.78 |
| | | | (br t, J = 4.9 Hz, 2H), 3.71 (br s, 1H), 2.22 (br t, J = 11.6 Hz, |
| | | | 1H), 2.02-1.88 (m, 2H), 1.81-1.67 (m, 2H), 1.38-1.18 (m, |
| | | | 4H) |
| 270 | 428 | 429 | δ 8.48 (d, J = 3.5 Hz, 1H), 8.20 (d, J = 8.2 Hz, 1H), 8.05 (t, J = |
| | | | 7.9 Hz, 1H), 7.93-7.87 (m, 1H), 7.76 (br d, J = 7.9 Hz, 1H), |
| | | | 7.27 (d, J = 7.7 Hz, 1H), 4.31 (s, 2H), 4.04 (s, 3H), 3.72-3.61 |
| | | | (m, 1H), 3.56-3.45 (m, 1H), 2.13-2.07 (m, 1H), 2.02-1.95 |
| | | | (m, 2H), 1.86-1.81 (m, 2H), 1.79 (s, 3H), 1.41-1.31 (m, 2H), |
| | | | 1.31-1.24 (m, 2H) |
| 271 | 414 | 415 | δ 8.47 (d, J = 3.5 Hz, 1H), 8.19 (d, J = 8.6 Hz, 1H), 8.04 (t, J = |
| | | | 8.0 Hz, 1H), 7.96-7.86 (m, 1H), 7.29-7.18 (m, 2H), 6.69 (br |
| | | | s, 1H), 4.30 (s, 2H), 4.03 (s, 4H), 3.74-3.59 (m, 1H), 2.12- |
| | | | 1.96 (m, 3H), 1.80 (br d, J = 11.8 Hz, 2H), 1.53-1.37 (m, 2H), |
| | | | 1.33-1.21 (m, 2H) |
| 272 | 428 | 429 | δ 8.69-8.54 (m, 2H), 8.52 (d, J = 3.4 Hz, 1H), 7.73 (br t, J = |
| | | | 7.4 Hz, 2H), 7.33 (d, J = 7.8 Hz, 1H), 4.30 (s, 2H), 4.01 (s, 4H), |
| | | | 3.65 (dt, J = 7.1, 3.3 Hz, 1H), 3.48 (dt, J = 7.3, 3.6 Hz, 1H), |
| | | | 1.96 (br d, J = 9.9 Hz, 2H), 1.85-1.79 (m, 2H), 1.78 (s, 3H), |
| | | | 1.39-1.19 (m, 4H) |
| 273 | 414 | 415 | δ 8.63 (d, J = 5.1 Hz, 2H), 8.52 (d, J = 3.3 Hz, 1H), 8.13 (s, |
| | | | 1H), 7.73 (br d, J = 3.8 Hz, 1H), 7.33 (br d, J = 7.9 Hz, 1H), |
| | | | 7.20 (br s, 1H), 6.70-6.65 (m, 1H), 4.30 (s, 2H), 4.01 (s, 4H), |
| | | | 3.64 (br dd, J = 7.1, 3.0 Hz, 1H), 2.10-2.03 (m, 1H), 2.03- |
| | | | 1.95 (m, 2H), 1.79 (br d, J = 11.4 Hz, 2H), 1.52-1.37 (m, 2H), |
| | | | 1.34-1.20 (m, 2H) |
| 274 | 412 | 413 | δ 8.44 (d, J = 3.6 Hz, 1H), 8.00-7.93 (m, 1H), 7.92-7.79 (m, |
| | | | 2H), 7.27-7.15 (m, 2H), 6.68 (br s, 1H), 3.93 (t, J = 5.8 Hz, |
| | | | 2H), 3.65 (br d, J = 2.3 Hz, 1H), 2.11-1.95 (m, 3H), 1.93- |
| | | | 1.68 (m, 7H), 1.53-1.36 (m, 2H), 1.34-1.09 (m, 3H) |
| 606 | 399 | 400 | δ = 8.41 (br d, J = 3.6 Hz, 1H), 7.55-7.45 (m, 1H), 7.38 (br d, |
| | | | J = 5.0 Hz, 2H), 7.30-7.22 (m, 1H), 7.13 (br dd, J = 2.9, 5.3 |
| | | | Hz, 1H), 4.35-4.20 (m, 1H), 3.92 (br dd, J = 3.2, 5.1 Hz, 1H), |
| | | | 3.85-3.66 (m, 5H), 3.15 (br d, J = 4.1 Hz, 5H), 2.80-2.64 (m, |
| | | | 1H), 2.06-1.95 (m, 3H), 1.94-1.82 (m, 2H), 1.50-1.25 (m, 2H) |
| 607 | 399 | 400 | δ (DMSO-d6) 8.39 (d, J = 3.8 Hz, 1H), 7.54 (br s, 1H), 7.51- |
| | | | 7.33 (m, 2H), 7.21 (br s, 1H), 7.17-7.10 (m, 2H), 6.68 (br s, |
| | | | 1H), 3.82-3.72 (m, 4H), 3.70-3.57 (m, 1H), 3.19-3.13 (m, |
| | | | 4H), 2.13-1.98 (m, 3H), 1.80 (br d, J = 11.6 Hz, 2H), 1.51- |
| | | | 1.35 (m, 2H), 1.34-1.21 (m, 2H) |
| 608 | 413 | 414 | δ (DMSO-d6) 8.43-8.35 (m, 1H), 7.77-7.70 (m, 1H), 7.59- |
| | | | 7.50 (m, 1H), 7.44-7.34 (m, 2H), 7.18-7.08 (m, 2H), 3.76 (br |
| | | | s, 4H), 3.19-3.12 (m, 4H), 3.05 (br s, 2H), 2.23 (br s, 1H), |
| | | | 2.03-1.92 (m, 2H), 1.82-1.75 (m, 4H), 1.37-1.20 (m, 4H) |
| 609 | 404 | 405 | δ (DMSO-d6) 8.45 (d, J = 3.6 Hz, 1H), 8.25 (br s, 1H), 7.96 (br |
| | | | d, J = 7.5 Hz, 1H), 7.84 (br d, J = 7.9 Hz, 1H), 7.75-7.70 (m, |
| | | | 3H), 7.67-7.62 (m, 1H), 7.52 (t, J = 7.6 Hz, 2H), 7.45-7.40 |
| | | | (m, 1H), 7.20 (br d, J = 7.6 Hz, 1H), 3.66 (br d, J = 6.6 Hz, 1H), |
| | | | 3.50 (br dd, J = 6.9, 3.3 Hz, 1H), 2.01-1.98 (m, 1H), 1.85- |
| | | | 1.77 (m, 5H), 1.40-1.20 (m, 5H) |
| 610 | 390 | 391 | δ (DMSO-d6) 8.45 (d, J = 3.6 Hz, 1H), 8.26 (br s, 1H), 7.97 (br |
| | | | d, J = 6.5 Hz, 1H), 7.85 (d, J = 7.8 Hz, 1H), 7.75-7.70 (m, |
| | | | 2H), 7.68-7.63 (m, 1H), 7.52 (t, J = 7.6 Hz, 2H), 7.45-7.40 |
| | | | (m, 1H), 7.21 (br d, J = 7.6 Hz, 2H), 6.69 (br s, 1H), 3.75-3.59 |
| | | | (m, 1H), 2.13-1.96 (m, 3H), 1.81 (br d, J = 12.3 Hz, 2H), 1.51- |
| | | | 1.38 (m, 2H), 1.34-1.23 (m, 2H) |
| 611 | 390 | 391 | δ (DMSO-d6) 8.48 (d, J = 3.6 Hz, 1H), 8.25 (s, 1H), 7.97 (br d, |
| | | | J = 7.8 Hz, 1H), 7.87-7.82 (m, 1H), 7.72 (d, J = 7.4 Hz, 2H), |
| | | | 7.68-7.63 (m, 1H), 7.52 (t, J = 7.6 Hz, 2H), 7.46-7.40 (m, |
| | | | 1H), 7.38-7.33 (m, 1H), 4.28 (br d, J = 13.6 Hz, 1H), 4.01- |
| | | | 3.91 (m, 1H), 3.83 (br d, J = 13.6 Hz, 1H), 3.40-3.34 (m, 1H), |
| | | | 3.22-3.12 (m, 1H), 2.76 (br t, J = 11.6 Hz, 1H), 2.02 (s, 3H), |
| | | | 1.94 (br d, J = 6.3 Hz, 1H), 1.52-1.31 (m, 2H) |
| 612 | 390 | 391 | δ (DMSO-d6) 8.45 (d, J = 3.7 Hz, 1H), 8.25 (s, 1H), 7.96 (br d, |
| | | | J = 7.4 Hz, 1H), 7.85 (d, J = 7.9 Hz, 1H), 7.74-7.62 (m, 3H), |
| | | | 7.52 (t, J = 7.6 Hz, 2H), 7.46-7.40 (m, 1H), 7.31-7.19 (m, |
| | | | 2H), 6.69 (br s, 1H), 3.74 (br d, J = 7.7 Hz, 1H), 2.24 (br t, J = |
| | | | 11.9 Hz, 1H), 2.07-1.94 (m, 2H), 1.85-1.68 (m, 2H), 1.42- |
| | | | 1.16 (m, 4H) |
| 613 | 393 | 394 | δ 8.51 (t, J = 3.6 Hz, 1H), 8.07 (br d, J = 7.9 Hz, 1H), 7.99 (br |
| | | | d, J = 7.4 Hz, 1H), 7.76-7.58 (m, 4H), 7.54 (ddd, J = 2.1, 6.8, |
| | | | 9.1 Hz, 1H), 6.51 (d, J = 9.3 Hz, 1H), 6.39-6.31 (m, 1H), 4.50- |
| | | | 4.26 (m, 1H), 3.85-3.40 (m, 3H), 3.29 (br s, 1H), 2.26-1.95 |
| | | | (m, 2H), 1.91 (d, J = 10.6 Hz, 3H) |
| 614 | 407 | 408 | δ 8.48 (d, J = 3.6 Hz, 1H), 8.05 (d, J = 7.1 Hz, 1H), 7.98 (s, |
| | | | 1H), 7.77-7.64 (m, 2H), 7.63-7.47 (m, 2H), 7.37 (d, J = 7.6 |
| | | | Hz, 1H), 6.51 (d, J = 9.1 Hz, 1H), 6.35 (dt, J = 1.3, 6.7 Hz, 1H), |
| | | | 4.25 (br d, J = 13.3 Hz, 1H), 4.03-3.86 (m, 1H), 3.79 (br d, J = |
| | | | 13.4 Hz, 1H), 3.22-3.07 (m, 1H), 2.75 (br t, J = 11.2 Hz, 1H), |
| | | | 2.06-1.81 (m, 5H), 1.54-1.22 (m, 2H) |
| 615 | 397 | 398 | δ 8.37 (d, J = 3.8 Hz, 1H), 7.68-7.45 (m, 1H), 7.35 (br s, 2H), |
| | | | 7.22-7.06 (m, 3H), 6.67 (br s, 1H), 3.62 (td, J = 3.7, 7.1 Hz, |
| | | | 1H), 3.37 (br s, 2H), 2.17-1.89 (m, 4H), 1.79 (br d, J = 11.1 |
| | | | Hz, 2H), 1.71-1.47 (m, 7H), 1.47-1.35 (m, 2H), 1.30-1.19 |
| | | | (m, 2H) |
| 616 | 41. | 412 | δ 8.37 (d, J = 3.9 Hz, 1H), 7.76-7.71 (m, 1H), 7.56-7.49 (m, |
| | | | 1H), 7.35-7.29 (m, 2H), 7.14-7.08 (m, 2H), 3.66-3.56 (m, |
| | | | 1H), 3.49 (br dd, J = 3.8, 7.6 Hz, 1H), 3.24-3.15 (m, 4H), 2.54 |
| | | | (s, 1H), 2.02-1.94 (m, 2H), 1.81 (br d, J = 12.3 Hz, 2H), 1.78 |
| | | | (s, 3H), 1.63 (br d, J = 4.6 Hz, 3H), 1.56 (br d, J = 4.8 Hz, 2H), |
| | | | 1.39-1.29 (m, 2H), 1.24 (br t, J = 12.1 Hz, 2H). |
| 617 | 408 | 409 | δ 8.50 (br d, J = 3.4 Hz, 1H), 8.24-8.06 (m, 2H), 8.01-7.90 |
| | | | (m, 2H), 7.57 (ddd, J = 2.1, 6.7, 9.1 Hz, 1H), 7.31 (br d, J = 8.0 |
| | | | Hz, 1H), 7.25-7.16 (m, 1H), 6.68 (br s, 1H), 6.55 (d, J = 9.3 |
| | | | Hz, 1H), 6.48-6.37 (m, 1H), 3.76-3.57 (m, 1H), 2.12-1.95 |
| | | | (m, 3H), 1.80 (br d, J = 12.1 Hz, 2H), 1.48-1.37 (m, 2H), 1.34- |
| | | | 1.23 (m, 2H) |
| 618 | 354 | 355 | δ 8.39 (d, J = 3.8 Hz, 1H), 7.74-7.66 (m, 2H), 7.40 (t, J = 8.0 |
| | | | Hz, 1H), 7.24 (br d, J = 7.8 Hz, 1H), 7.19 (br s, 1H), 7.13 (d, J = |
| | | | 7.8 Hz, 1H), 6.67 (br s, 1H), 3.70-3.56 (m, 1H), 2.11-1.94 |
| | | | (m, 4H), 1.80 (br d, J = 11.8 Hz, 2H), 1.48-1.37 (m, 2H), 1.33- |
| | | | 1.21 (m, 2H), 1.04-0.96 (m, 2H), 0.75-0.66 (m, 2H) |
| 619 | 368 | 369 | δ 8.41 (d, J = 3.7 Hz, 1H), 7.80-7.67 (m, 3H), 7.42 (t, J = 7.6 |
| | | | Hz, 1H), 7.30-7.24 (m, 1H), 7.17 (br d, J = 7.5 Hz, 1H), 3.67- |
| | | | 3.60 (m, 1H), 3.58-3.45 (m, 1H), 2.09-1.93 (m, 3H), 1.88- |
| | | | 1.81 (m, 2H), 1.79 (s, 3H), 1.42-1.21 (m, 4H), 1.06-0.98 (m, |
| | | | 2H), 0.77-0.68 (m, 2H) |
| 620 | 385 | 386 | δ 8.14 (d, J = 6.1 Hz, 1H), 7.17 (br d, J = 8.1 Hz, 3H), 7.09- |
| | | | 6.92 (m, 1H), 6.69-6.62 (m, 1H), 4.87 (br d, J = 8.0 Hz, 2H), |
| | | | 3.53 (br dd, J = 1.9, 4.8 Hz, 1H), 2.35-2.25 (m, 2H), 2.08- |
| | | | 1.98 (m, 1H), 1.97-1.83 (m, 6H), 1.80-1.72 (m, 2H), 1.40 (br |
| | | | d, J = 12.5 Hz, 2H), 1.23 (br s, 2H). |
| 621 | 414 | 415 | δ 8.45 (d, J = 3.6 Hz 1H,), 7.9-8.0 (m, 2H), 7.84 (br s, 1H), 7.20 |
| | | | (br s, 2H), 6.67 (br s, 1H), 4.36 (t, J = 5.4 Hz 2H,), 3.99 (t, J = 6.2 |
| | | | Hz 2H,), 3.6-3.7 (m, 1H), 1.9-2.2 (m, 5H), 1.79 (br d, J = 11.3 |
| | | | Hz, 2H), 1.4-1.5 (m, 2H), 1.2-1.4 (m, 2H) |
| 622 | 414 | 415 | δ 8.67-8.49 (m, 2H), 8.40 (br d, J = 8.9 Hz, 1H), 7.68 (br d, J = |
| | | | 3.5 Hz, 1H), 7.33 (br d, J = 7.9 Hz, 1H), 7.20 (br s, 1H), 6.68 |
| | | | (br s, 1H), 4.38 (t, J = 5.4 Hz, 2H), 3.97 (t, J = 6.2 Hz, 2H), |
| | | | 3.75-3.52 (m, 1H), 2.22-1.94 (m, 5H), 1.79 (br d, J = 12.0 |
| | | | Hz, 2H), 1.52-1.16 (m, 4H) |
| 623 | 408 | 409 | δ = 8.79 (d, J = 5.3 Hz, 1H), 8.55 (d, J = 3.3 Hz, 1H), 8.29 (s, |
| | | | 1H), 7.99 (br d, J = 4.9 Hz, 1H), 7.95 (br d, J = 5.5 Hz, 1H), |
| | | | 7.56 (ddd, J = 2.1, 6.7, 9.1 Hz, 1H), 7.38 (br d, J = 8.0 Hz, 1H), |
| | | | 7.19 (br s, 1H), 6.68 (br s, 1H), 6.54 (d, J = 9.1 Hz, 1H), 6.40 |
| | | | (dt, J = 1.1, 6.8 Hz, 1H), 3.71-3.59 (m, 1H), 2.10-1.94 (m, |
| | | | 3H), 1.78 (br d, J = 12.0 Hz, 2H), 1.49-1.36 (m, 2H), 1.34- |
| | | | 1.21 (m, 2H) |
| 624 | 428 | 429 | δ (DMSO-d6) 8.47 (br s, 1H), 7.99 (br s, 2H), 7.88-7.71 (m, |
| | | | 2H), 7.24 (br d, J = 4.7 Hz, 1H), 4.38 (br d, J = 3.7 Hz, 2H), |
| | | | 4.06-3.95 (m, 2H), 3.73-3.60 (m, 1H), 3.57-3.44 (m, 1H), |
| | | | 2.15 (br s, 2H), 1.98 (br s, 2H), 1.86-1.76 (m, 5H), 1.39-1.21 |
| | | | (m, 4H) |
| 625 | 426 | 427 | δ (DMSO-d6) 8.45 (d, J = 3.5 Hz, 1H), 8.00-7.94 (m, 1H), |
| | | | 7.92-7.88 (m, 1H), 7.82 (br d, J = 5.5 Hz, 1H), 7.73 (d, J = 7.9 |
| | | | Hz, 1H), 7.22 (d, J = 7.9 Hz, 1H), 3.93 (t, J = 5.8 Hz, 2H), 3.72- |
| | | | 3.59 (m, 1H), 3.55-3.43 (m, 1H), 1.98 (br d, J = 10.5 Hz, |
| | | | 2H), 1.92-1.79 (m, 7H), 1.78 (s, 3H), 1.42-1.17 (m, 5H) |
| 626 | 422 | 423 | δ (DMSO-d6) 8.51 (d, J = 3.4 Hz, 1H), 8.23-8.18 (m, 1H), |
| | | | 8.10 (br d, J = 6.8 Hz, 1H), 8.00-7.94 (m, 2H), 7.75 (br d, J = |
| | | | 7.8 Hz, 1H), 7.58 (ddd, J = 9.1, 6.7, 2.1 Hz, 1H), 7.32 (br d, J = |
| | | | 7.8 Hz, 1H), 6.57 (d, J = 8.9 Hz, 1H), 6.43 (dt, J = 6.8, 1.2 Hz, |
| | | | 1H), 3.73-3.62 (m, 1H), 3.56-3.46 (m, 1H), 1.99 (br d, J = |
| | | | 10.5 Hz, 2H), 1.84 (br d, J = 11.9 Hz, 2H), 1.79 (s, 3H), 1.41- |
| | | | 1.30 (m, 3H), 1.26 (br d, J = 13.3 Hz, 1H) |
| 627 | 414 | 415 | δ (DMSO-d6) 8.47 (d, J = 3.5 Hz, 1H), 8.19 (d, J = 8.3 Hz, 1H), |
| | | | 8.04 (t, J = 7.9 Hz, 1H), 7.89 (br d, J = 6.9 Hz, 1H), 7.37-7.16 |
| | | | (m, 2H), 6.69 (br s, 1H), 4.30 (s, 2H), 4.03 (s, 4H), 3.85-3.67 |
| | | | (m, 1H), 2.23 (br t, J = 11.9 Hz, 1H), 2.06-1.89 (m, 2H), 1.85- |
| | | | 1.68 (m, 2H), 1.43-1.14 (m, 4H) |
| 628 | 414 | 415 | δ (DMSO-d6) 8.76 (br d, J = 2.1 Hz, 1H), 8.63 (d, J = 5.3 Hz, |
| | | | 1H), 8.54 (d, J = 3.5 Hz, 1H), 7.78-7.69 (m, 1H), 7.48 (d, J = |
| | | | 7.5 Hz, 1H), 4.30 (s, 2H), 4.26 (br s, 1H), 4.01 (s, 4H), 3.97- |
| | | | 3.89 (m, 1H), 3.81 (br d, J = 13.8 Hz, 1H), 3.15 (br t, J = 11.5 |
| | | | Hz, 1H), 2.74 (br t, J = 11.7 Hz, 1H), 2.00 (s, 3H), 1.98-1.84 |
| | | | (m, 2H), 1.49-1.27 (m, 2H) |
| 629 | 414 | 415 | δ (DMSO-d6) 8.75-8.55 (m, 2H), 8.51 (d, J = 3.5 Hz, 1H), |
| | | | 7.72 (br d, J = 4.3 Hz, 1H), 7.40 (br d, J = 7.0 Hz, 1H), 7.20 (br |
| | | | s, 1H), 6.67 (br s, 1H), 4.30 (s, 2H), 4.01 (s, 4H), 3.79-3.65 |
| | | | (m, 1H), 2.23 (br t, J = 11.6 Hz, 1H), 2.05-1.86 (m, 2H), 1.82- |
| | | | 1.65 (m, 2H), 1.39-1.15 (m, 4H) |
| 630 | 414 | 415 | δ (DMSO-d6) 8.50 (d, J = 3.4 Hz, 1H), 8.20 (d, J = 8.3 Hz, 1H), |
| | | | 8.05 (t, J = 7.9 Hz, 1H), 7.93 (br s, 1H), 7.41 (br d, J = 7.5 Hz, |
| | | | 1H), 4.31 (s, 3H), 4.04 (s, 4H), 3.99-3.92 (m, 1H), 3.83 (br d, |
| | | | J = 13.4 Hz, 1H), 3.17 (br t, J = 11.6 Hz, 1H), 2.76 (br t, J = |
| | | | 11.6 Hz, 1H), 2.02 (s, 3H), 1.99-1.88 (m, 2H), 1.50-1.43 (m, |
| | | | 1H), 1.37-1.29 (m, 1H) |
| 631 | 425 | 426 | δ 8.41 (d, J = 3.5 Hz, 1H), 7.89 (br s, 1H), 7.80 (br d, J = 2.7 |
| | | | Hz, 1H), 7.75 (br d, J = 7.7 Hz, 1H), 7.61 (br d, J = 3.1 Hz, |
| | | | 1H), 7.54-7.45 (m, 2H), 7.18 (br d, J = 7.8 Hz, 1H), 3.70- |
| | | | 3.59 (m, 1H), 3.55-3.43 (m, 1H), 3.31 (br s, 1H), 3.27-3.18 |
| | | | (m, 1H), 3.13-3.02 (m, 1H), 2.40-2.24 (m, 2H), 1.97 (br s, |
| | | | 4H), 1.81 (br d, J = 12.8 Hz, 2H), 1.77 (s, 3H), 1.39-1.17 (m, |
| | | | 4H) |

TABLE 4
Spectrometric data for compounds of Table 2

| Cmpd | MS | MS | |
|---|---|---|---|
| No. | Calc. | found | ¹H NMR Data (400 MHZ, DMSO-d₆) |
| 44 | 843 | 844 | δ = 11.10 (s, 1H), 8.52-8.45 (m, 1H), 8.01 (br d, J = 2.0 Hz, 1H), |
| | | | 7.87-7.60 (m, 6H), 7.55-7.48 (m, 2H), 7.45-7.39 (m, 1H), 7.11- |
| | | | 7.04 (m, 2H), 6.94 (br d, J = 7.9 Hz, 1H), 5.36 (br dd, J = 5.3, |
| | | | 12.8 Hz, 1H), 3.61 (br t, J = 6.4 Hz, 8H), 3.35 (s, 3H), 3.19-3.03 |
| | | | (m, 4H), 2.97-2.84 (m, 4H), 2.77-2.60 (m, 4H), 2.09-1.98 (m, |
| | | | 4H), 1.95-1.85 (m, 3H), 1.76 (td, J = 3.2, 6.5 Hz, 7H), 1.67- |
| | | | 1.58 (m, 1H) |
| 45 | 843 | 844 | δ = 11.10 (s, 1H), 8.46 (br s, 1H), 8.00 (br s, 1H), 7.82 (br d, J = |
| | | | 7.6 Hz, 1H), 7.78-7.67 (m, 3H), 7.66-7.56 (m, 2H), 7.51 (br t, |
| | | | J = 7.6 Hz, 2H), 7.44-7.38 (m, 1H), 7.15-7.00 (m, 2H), 6.97- |
| | | | 6.84 (m, 1H), 5.42-5.30 (m, 1H), 4.40-4.23 (m, 3H), 4.04-3.89 |
| | | | (m, 4H), 3.35 (br s, 3H), 3.27-3.15 (m, 4H), 2.88-2.65 (m, 9H), |
| | | | 2.13-1.70 (m, 12H), 1.62-1.18 (m, 3H) |
| 46 | 857 | 858 | δ = 11.08 (s, 1H), 8.46 (s, 1H), 8.15 (s, 1H), 8.00 (br s, 1H), 7.81 |
| | | | (d, J = 7.8 Hz, 1H), 7.76-7.69 (m, 3H), 7.61 (br t, J = 7.8 Hz, |
| | | | 2H), 7.51 (t, J = 7.6 Hz, 2H), 7.45-7.38 (m, 1H), 7.09-7.00 (m, |
| | | | 2H), 6.91 (br d, J = 7.9 Hz, 1H), 5.34 (dd, J = 5.4, 12.6 Hz, 1H), |
| | | | 4.51 (br d, J = 12.0 Hz, 1H), 4.29 (br d, J = 8.1 Hz, 1H), 4.16 (br |
| | | | d, J = 11.3 Hz, 1H), 4.03-3.90 (m, 2H), 3.33-3.31 (m, 3H), 3.23- |
| | | | 3.02 (m, 4H), 2.97-2.81 (m, 4H), 2.72-2.58 (m, 4H), 2.15 (br |
| | | | s, 2H), 2.06-1.75 (m, 6H), 1.71-1.55 (m, 5H), 1.53-1.40 (m, |
| | | | 2H), 1.33 (br d, J = 9.9 Hz, 1H) |
| 47 | 843 | 844 | δ = 11.10 (s, 1H), 9.64-9.09 (m, 1H), 8.47 (s, 1H), 8.00 (br s, |
| | | | 1H), 7.82 (td, J = 1.4, 7.8 Hz, 1H), 7.78-7.69 (m, 3H), 7.69- |
| | | | 7.59 (m, 2H), 7.51 (t, J = 7.6 Hz, 2H), 7.45-7.39 (m, 1H), 7.05 |
| | | | (br d, J = 5.9 Hz, 1H), 6.92 (br d, J = 8.1 Hz, 1H), 5.35 (br dd, |
| | | | J = 5.3, 12.7 Hz, 1H), 4.39-4.19 (m, 2H), 4.03 (br d, J = 2.8 Hz, |
| | | | 2H), 3.24-3.11 (m, 3H), 3.02-2.83 (m, 7H), 2.77-2.63 (m, 5H), |
| | | | 2.22-1.65 (m, 14H), 1.64-1.23 (m, 5H) |
| 48 | 788 | 789 | δ = 10.78 (s, 1H), 9.74 (s, 1H), 9.41 (s, 1H), 8.46 (s, 1H), 8.00 (s, |
| | | | 1H), 7.81 (d, J = 7.8 Hz, 1H), 7.71 (d, J = 7.3 Hz, 3H), 7.67-7.58 |
| | | | (m, 2H), 7.50 (t, J = 7.6 Hz, 2H), 7.45-7.36 (m, 1H), 6.95 (d, J = |
| | | | 8.4 Hz, 2H), 6.64 (d, J = 8.5 Hz, 2H), 4.28 (d, J = 4.6, 11.1 Hz, |
| | | | 2H), 4.08-3.95 (m, 1H), 3.92-3.71 (m, 3H), 3.22-2.97 (m, 6H), |
| | | | 2.92-2.81 (m, 4H), 2.79-2.65 (m, 2H), 2.63-2.52 (m, 1H), 2.29 |
| | | | (d, J = 12.0 Hz, 2H), 2.17-2.05 (m, 2H), 2.02-1.80 (m, 9H), |
| | | | 1.60-1.27 (m, 3H) |
| 49 | 843 | 844 | δ 11.09 (br s, 1H), 8.45-8.40 (m, 1H), 8.08-7.96 (m, 1H), 7.84 |
| | | | (br s, 1H), 7.72 (d, J = 7.6 Hz, 2H), 7.66-7.57 (m, 1H), 7.51 (br |
| | | | t, J = 7.6 Hz, 3H), 7.44-7.39 (m, 1H), 7.10 (s, 1H), 7.03-6.97 |
| | | | (m, 1H), 6.93-6.87 (m, 1H), 5.34 (dd, J = 5.6, 12.5 Hz, 1H), 3.87- |
| | | | 3.77 (m, 1H), 3.40 (br d, J = 3.2 Hz, 6H), 3.34 (br s, 3H), 3.33- |
| | | | 3.29 (m, 2H), 3.02-2.86 (m, 3H), 2.80-2.58 (m, 4H), 2.46 (br |
| | | | s, 2H), 2.37-2.33 (m, 2H), 2.13-1.89 (m, 6H), 1.79-1.60 (m, |
| | | | 6H), 1.51-1.34 (m, 2H), 1.32-1.22 (m, 2H) |
| 50 | 828 | 829 | δ = 10.98 (s, 1H), 8.45 (s, 1H), 8.00 (br s, 1H), 7.81 (td, J = 1.4, |
| | | | 7.8 Hz, 1H), 7.78-7.67 (m, 3H), 7.67-7.56 (m, 3H), 7.55-7.46 |
| | | | (m, 3H), 7.45-7.34 (m, 2H), 5.10 (dd, J = 5.0, 13.3 Hz, 1H), 4.49- |
| | | | 4.22 (m, 4H), 4.07-3.92 (m, 3H), 3.11 (br t, J = 11.5 Hz, 4H), |
| | | | 3.02-2.92 (m, 3H), 2.91-2.81 (m, 3H), 2.70-2.61 (m, 2H), 2.22 |
| | | | (br d, J = 6.6 Hz, 2H), 2.09-1.99 (m, 4H), 1.96-1.86 (m, 2H), |
| | | | 1.82-1.75 (m, 2H), 1.71 (br d, J = 9.4 Hz, 3H), 1.59-1.45 (m, |
| | | | 2H), 1.40-1.28 (m, 1H), 1.25-1.08 (m, 2H) |
| 51 | 857 | 858 | δ 11.09 (s, 1H), 8.48-8.37 (m, 1H), 7.99 (br s, 1H), 7.81 (br d, |
| | | | J = 7.6 Hz, 1H), 7.76-7.69 (m, 3H), 7.65-7.57 (m, 1H), 7.50 (br |
| | | | t, J = 7.6 Hz, 3H), 7.43-7.38 (m, 1H), 7.08 (br s, 1H), 7.01 (br |
| | | | d, J = 8.0 Hz, 1H), 6.94-6.85 (m, 1H), 5.34 (dd, J = 13.0, 5.3 Hz, |
| | | | 1H), 3.91-3.78 (m, 1H), 3.58-3.48 (m, 5H), 3.47-3.40 (m, 3H), |
| | | | 3.08-2.78 (m, 5H), 2.73-2.58 (m, 3H), 2.36-2.05 (m, 3H), 2.03- |
| | | | 1.91 (m, 3H), 1.84-1.68 (m, 6H), 1.67-1.55 (m, 2H), 1.48- |
| | | | 1.36 (m, 2H), 1.33-1.17 (m, 4H) |
| 52 | 842 | 843 | δ 11.08 (s, 1H), 8.42 (br s, 1H), 8.29 (s, 1H), 7.99 (br s, 1H), 7.81 |
| | | | (br d, J = 7.9 Hz, 2H), 7.71 (br d, J = 7.3 Hz, 3H), 7.65-7.57 (m, |
| | | | 1H), 7.55-7.45 (m, 4H), 7.44-7.36 (m, 2H), 7.15-6.79 (m, 4H), |
| | | | 5.34 (dd, J = 12.6, 5.4 Hz, 1H), 4.42-4.26 (m, 2H), 3.95-3.77 |
| | | | (m, 6H), 2.93-2.85 (m, 1H), 2.86-2.84 (m, 1H), 2.75-2.74 (m, |
| | | | 1H), 2.73-2.63 (m, 4H), 2.38-2.08 (m, 5H), 2.06-1.82 (m, 5H), |
| | | | 1.66-1.49 (m, 6H), 1.28-1.25 (m, 1H), 1.09-0.75 (m, 5H) |
| 53 | 788 | 789 | δ 10.78 (s, 1H), 8.47 (s, 1H), 8.14 (s, 0.32H), 8.00 (br s, 1H), 7.82 |
| | | | (d, J = 7.9 Hz, 1H), 7.72 (br d, J = 7.4 Hz, 3H), 7.66-7.59 (m, |
| | | | 2H), 7.51 (t, J = 7.6 Hz, 2H), 7.41 (s, 1H), 6.96 (br d, J = 8.3 Hz, |
| | | | 2H), 6.63 (d, J = 8.4 Hz, 2H), 5.71 (br d, J = 7.4 Hz, 1H), 4.35- |
| | | | 4.16 (m, 2H), 4.10-3.79 (m, 3H), 3.20-3.10 (m, 3H), 2.94-2.80 |
| | | | (m, 2H), 2.79-2.65 (m, 3H), 2.58 (br dd, J = 17.6, 4.0 Hz, 3H), |
| | | | 2.17-2.05 (m, 2H), 2.04-1.61 (m, 13H), 1.58-1.22 (m, 5H) |
| 54 | 842 | 843 | δ 10.99 (s, 1H), 8.42 (br s, 1H), 7.99 (br d, J = 1.0 Hz, 1H), 7.81 |
| | | | (br d, J = 7.6 Hz, 1H), 7.77-7.65 (m, 4H), 7.62 (br d, J = 6.6 Hz, |
| | | | 1H), 7.50 (br t, J = 7.6 Hz, 4H), 7.45-7.38 (m, 2H), 5.11 (dd, J = |
| | | | 13.2, 5.1 Hz, 1H), 4.47-4.38 (m, 1H), 4.35-4.25 (m, 1H), 3.92- |
| | | | 3.76 (m, 1H), 3.65-3.40 (m, 8H), 3.28-3.05 (m, 2H), 3.01- |
| | | | 2.69 (m, 4H), 2.60 (br d, J = 16.4 Hz, 2H), 2.39 (qd, J = 13.1, 3.7 |
| | | | Hz, 2H), 2.07-1.78 (m, 8H), 1.74-1.52 (m, 2H), 1.51-1.37 (m, |
| | | | 2H), 1.34-1.19 (m, 2H) |
| 55 | 788 | 789 | δ 10.79 (s, 1H), 8.43 (s, 1H), 8.05-7.95 (m, 1H), 7.82 (br d, J = |
| | | | 7.7 Hz, 1H), 7.72 (br d, J = 7.4 Hz, 3H), 7.65-7.59 (m, 1H), 7.51 |
| | | | (br t, J = 7.5 Hz, 4H), 7.45-7.39 (m, 1H), 6.97 (br d, J = 8.5 Hz, |
| | | | 2H), 6.65 (d, J = 8.5 Hz, 2H), 4.29 (dd, J = 11.3, 4.6 Hz, 1H), |
| | | | 3.90-3.80 (m, 2H), 3.09 (br dd, J = 4.5, 2.1 Hz, 4H), 2.87-2.58 |
| | | | (m, 9H), 2.16-2.04 (m, 2H), 1.99-1.73 (m, 10H), 1.67-1.58 |
| | | | (m, 1H), 1.52-1.35 (m, 3H), 1.33-1.12 (m, 4H) |
| 56 | 842 | 843 | δ 10.99 (s, 1H), 9.56-9.36 (m, 1H), 8.47 (s, 1H), 8.00 (br s, |
| | | | 0.9H), 7.82 (br d, J = 7.4 Hz, 1H), 7.77-7.67 (m, 4H), 7.62 (br t, |
| | | | J = 7.7 Hz, 2H), 7.56-7.48 (m, 3H), 7.42 (br t, J = 6.8 Hz, 2H), |
| | | | 5.11 (dd, J = 13.4, 4.8 Hz, 1H), 4.55 (br d, J = 12.6 Hz, 1H), 4.48- |
| | | | 4.41 (m, 1H), 4.37-4.24 (m, 3H), 4.06-3.90 (m, 2H), 3.87- |
| | | | 3.71 (m, 1H), 3.09-2.86 (m, 6H), 2.85-2.74 (m, 2H), 2.70-2.56 |
| | | | (m, 2H), 2.43-2.31 (m, 2H), 2.07-1.78 (m, 9H), 1.77-1.13 (m, |
| | | | 7H |
| 57 | 802 | 803 | δ 10.79 (s, 1H), 8.44 (br s, 1H), 8.00 (br s, 1H), 7.82 (br d, J = 7.6 |
| | | | Hz, 1H), 7.73 (br d, J = 7.5 Hz, 3H), 7.67-7.59 (m, 1H), 7.52 (br |
| | | | t, J = 7.6 Hz, 3H), 7.49-7.48 (m, 1H), 7.46-7.38 (m, 1H), 7.03- |
| | | | 6.90 (m, 2H), 6.64 (br d, J = 8.3 Hz, 2H), 5.78-5.68 (m, 1H), |
| | | | 4.34-4.25 (m, 1H), 3.90-3.80 (m, 1H), 3.63-3.37 (m, 10H), |
| | | | 2.84-2.67 (m, 3H), 2.65-2.55 (m, 2H), 2.11 (td, J = 8.7, 4.3 Hz, |
| | | | 1H), 2.00-1.76 (m, 9H), 1.65 (br d, J = 9.3 Hz, 2H), 1.50-1.37 |
| | | | (m, 2H), 1.35-1.19 (m, 3H) |
| 58 | 857 | 858 | δ 11.09 (s, 1H), 8.46-8.39 (m, 1H), 8.01 (br dd, J = 5.1, 2.1 Hz, |
| | | | 1H), 7.81 (br d, J = 7.4 Hz, 1H), 7.71 (br d, J = 7.4 Hz, 3H), 7.65- |
| | | | 7.58 (m, 1H), 7.50 (br t, J = 7.6 Hz, 3H), 7.44-7.38 (m, 1H), |
| | | | 7.11-7.08 (m, 1H), 7.07 (s, 1H), 7.06-7.05 (m, 1H), 7.03 (br d, |
| | | | J = 1.6 Hz, 1H), 6.91 (br d, J = 7.9 Hz, 2H), 5.34 (br dd, J = 12.9, |
| | | | 5.7 Hz, 1H), 4.57-4.48 (m, 1H), 3.89-3.76 (m, 2H), 3.28-3.23 |
| | | | (m, 2H), 3.20-3.01 (m, 3H), 2.96-2.73 (m, 5H), 2.72-2.57 (m, |
| | | | 5H), 2.04-1.74 (m, 8H), 1.72-1.57 (m, 3H), 1.54-1.35 (m, 4H), |
| | | | 1.31-1.17 (m, 3H) |
| 59 | 856 | 857 | δ 11.11 (s, 1H), 8.43 (br s, 1H), 7.99 (br s, 1H), 7.81 (br d, J = 7.7 |
| | | | Hz, 1H), 7.72 (br d, J = 7.6 Hz, 3H), 7.64-7.59 (m, 1H), 7.51 (br |
| | | | t, J = 7.5 Hz, 3H), 7.43 (br d, J = 7.2 Hz, 1H), 7.11 (br s, 1H), |
| | | | 7.03 (br d, J = 7.7 Hz, 1H), 6.95-6.85 (m, 1H), 5.35 (dd, J = 12.7, |
| | | | 5.2 Hz, 1H), 4.65-4.49 (m, 1H), 4.45-4.29 (m, 1H), 3.34 (s, |
| | | | 3H), 3.17-2.83 (m, 3H), 2.82-2.66 (m, 3H), 2.63 (br d, J = 18.0 |
| | | | Hz, 2H), 2.47 (br d, J = 3.3 Hz, 2H), 2.37-2.13 (m, 2H), 2.06- |
| | | | 1.85 (m, 4H), 1.77 (br d, J = 9.2 Hz, 4H), 1.71-1.35 (m, 7H), |
| | | | 1.34-1.17 (m, 2H), 1.15-0.87 (m, 2H) |
| 60 | 827 | 828 | δ 10.98 (s, 1H), 8.42 (br s, 1H), 7.99 (br s, 1H), 7.83-7.77 (m, |
| | | | 1H), 7.71 (br d, J = 7.0 Hz, 4H), 7.64-7.58 (m, 1H), 7.54-7.47 |
| | | | (m, 4H), 7.41 (br d, J = 7.4 Hz, 2H), 5.11 (dd, J = 13.1, 5.1 Hz, |
| | | | 1H), 4.57 (br d, J = 10.4 Hz, 1H), 4.46-4.37 (m, 2H), 4.34-4.27 |
| | | | (m, 1H), 4.21-3.91 (m, 2H), 3.83 (br d, J = 6.4 Hz, 1H), 3.20- |
| | | | 3.00 (m, 2H), 2.98-2.87 (m, 3H), 2.80-2.71 (m, 1H), 2.60 (br |
| | | | d, J = 16.0 Hz, 3H), 2.40 (br dd, J = 12.6, 3.9 Hz, 1H), 2.02-1.92 |
| | | | (m, 3H), 1.87-1.75 (m, 3H), 1.62 (br s, 3H), 1.50-1.22 (m, 8H) |
| 61 | 828 | 829 | δ 11.14-10.97 (m, 1H), 8.42 (br s, 1H), 7.99 (br s, 1H), 7.80 (br |
| | | | s, 1H), 7.67 (br s, 3H), 7.65-7.58 (m, 1H), 7.55-7.47 (m, 3H), |
| | | | 7.43-7.36 (m, 1H), 7.11 (br s, 1H), 7.02 (br d, J = 7.8 Hz, 1H), |
| | | | 6.95-6.84 (m, 1H), 5.44-5.04 (m, 1H), 4.56 (br d, J = 9.6 Hz, |
| | | | 1H), 4.48-4.27 (m, 1H), 4.19-3.92 (m, 2H), 3.83 (br d, J = 1.8 |
| | | | Hz, 1H), 2.96-2.85 (m, 2H), 2.82-2.67 (m, 3H), 2.66-2.55 (m, |
| | | | 3H), 2.44-2.31 (m, 1H), 2.04-1.91 (m, 3H), 1.89-1.71 (m, 4H), |
| | | | 1.62 (br s, 3H), 1.53-1.35 (m, 4H), 1.34-1.14 (m, 3H) |
| 62 | 827 | 828 | δ 9.96-9.85 (m, 1H), 8.93 (br s, 1H), 8.66 (br d, J = 1.7 Hz, 1H), |
| | | | 8.53 (br s, 1H), 8.40 (s, 1H), 7.85-7.78 (m, 2H), 7.75 (d, J = 7.3 |
| | | | Hz, 2H), 7.64 (t, J = 8.0 Hz, 1H), 7.57-7.46 (m, 3H), 7.45-7.38 |
| | | | (m, 1H), 7.04 (s, 1H), 3.91 (s, 2H), 2.56 (d, J = 7.8 Hz, 2H), 2.13 |
| | | | (br t, J = 7.5 Hz, 2H) |
| 63 | 813 | 814 | δ 10.98 (s, 1H), 8.42 (br s, 1H), 8.07-7.94 (m, 1H), 7.81 (br d, |
| | | | J = 8.4 Hz, 1H), 7.71 (br d, J = 7.4 Hz, 3H), 7.67-7.60 (m, 2H), |
| | | | 7.53-7.48 (m, 4H), 7.43-7.38 (m, 2H), 5.08 (br s, 1H), 4.44- |
| | | | 4.28 (m, 3H), 4.00-3.78 (m, 2H), 3.10-2.75 (m, 6H), 2.03-1.92 |
| | | | (m, 4H), 1.90-1.64 (m, 10H), 1.63-1.52 (m, 2H), 1.51-1.19 |
| | | | (m, 6H), 1.14-0.73 (m, 3H) |
| 64 | 787 | 788 | δ 10.78 (s, 1H), 8.42 (br s, 1H), 8.02-7.95 (m, 1H), 7.81 (br d, |
| | | | J = 7.5 Hz, 1H), 7.72 (br d, J = 7.4 Hz, 3H), 7.65-7.58 (m, 1H), |
| | | | 7.51 (br t, J = 7.3 Hz, 3H), 7.45-7.39 (m, 1H), 6.95 (br d, J = 7.0 |
| | | | Hz, 2H), 6.64 (br d, J = 8.4 Hz, 2H), 5.76 (d, J = 7.6 Hz, 1H), |
| | | | 4.42-4.33 (m, 1H), 4.32-4.24 (m, 1H), 4.02-3.76 (m, 2H), 3.54 |
| | | | (m, 1H), 3.12-2.93 (m, 4H), 2.78-2.68 (m, 2H), 2.63-2.55 (m, |
| | | | 2H), 2.13-2.06 (m, 1H), 2.00-1.82 (m, 6H), 1.81-1.66 (m, 5H), |
| | | | 1.59 (br d, J = 4.0 Hz, 4H), 1.49-1.36 (m, 3H), 1.33-1.18 (m, |
| | | | 3H), 1.13-0.84 (m, 2H) |
| 65 | 773 | 774 | δ 10.78 (s, 1H), 8.42 (br s, 1H), 8.35 (s, 1H), 8.14 (s, 1H), 8.03- |
| | | | 7.94 (m, 1H) 7.81 (br d, J = 7.3 Hz, 1H), 7.72 (br d, J = 7.4 Hz, |
| | | | 3H), 7.65-7.58 (m, 1H), 7.51 (br t, J = 7.4 Hz, 3H), 7.45-7.38 |
| | | | (m, 1H), 6.96 (br d, J = 7.4 Hz, 2H), 6.63 (br d, J = 8.1 Hz, 2H), |
| | | | 5.72 (br d, J = 7.5 Hz, 1H), 4.43-4.34 (m, 1H), 4.32-4.24 (m, |
| | | | 1H), 4.06-3.74 (m, 3H), 2.80-2.69 (m, 3H), 2.61 (br t, J = 4.1 |
| | | | Hz, 1H), 2.58-2.55 (m, 1H), 2.14-2.07 (m, 1H), 2.03-1.64 (m, |
| | | | 14H), 1.59 (br s, 2H), 1.48-1.21 (m, 6H), 1.04-0.82 (m, 2H) |
| 66 | 847 | 848 | δ 11.13 (s, 1H), 8.46 (br s, 1H), 8.01 (br s, 1H), 7.93 (br t, J = 5.4 |
| | | | Hz, 1H), 7.84-7.80 (m, 2H), 7.72 (br d, J = 7.5 Hz, 3H), 7.64- |
| | | | 7.59 (m, 2H), 7.53-7.49 (m, 3H), 7.44-7.39 (m, 2H), 5.13 (br |
| | | | dd, J = 12.9, 5.3 Hz, 1H), 4.78 (s, 2H), 4.34-4.26 (m, 1H), 4.02- |
| | | | 3.95 (m, 1H), 3.90-3.83 (m, 1H), 3.18-3.12 (m, 3H), 2.95- |
| | | | 2.87 (m, 1H), 2.78-2.71 (m, 1H), 2.65-2.55 (m, 3H), 2.30 (br t, |
| | | | J = 7.3 Hz, 2H), 2.09-2.03 (m, 1H), 1.96-1.87 (m, 2H), 1.49- |
| | | | 1.41 (m, 5H), 1.26 (br s, 11H) |
| 67 | 846 | 847 | δ 10.89 (s, 1H), 8.37 (br s, 1H), 8.06-8.00 (m, 1H), 7.91 (br s, |
| | | | 1H), 7.72 (d, J = 7.8 Hz, 1H), 7.68-7.60 (m, 3H), 7.59-7.47 (m, |
| | | | 3H), 7.45-7.38 (m, 2H), 7.35-7.29 (m, 1H), 7.08 (d, J = 1.8 Hz, |
| | | | 1H), 7.01 (m, 1H), 4.99 (m, 1H), 4.49 (s, 2H), 4.36-4.27 (m, |
| | | | 2H), 4.25-4.02 (m, 3H), 3.90 (m, 2H), 3.79-3.69 (m, 1H), 3.09 |
| | | | (br t, J = 11.8 Hz, 2H), 2.97 (br t, J = 11.8 Hz, 1H), 2.87-2.77 |
| | | | (m, 2H), 2.71-2.62 (m, 1H), 2.60-2.45 (m, 3H), 2.30 (m, 2H), |
| | | | 1.94-1.85 (m, 2H), 1.81 (br d, J = 11.3 Hz, 1H), 1.58-1.49 (m, |
| | | | 2H), 1.47-1.46 (m, 1H), 1.37-1.33 (m, 1H), 1.31-1.18 (m, 2H) |
| 68 | 860 | 861 | δ 10.97 (s, 1H), 8.46 (br s, 1H), 8.23-8.18 (m, 1H), 8.00 (br s, |
| | | | 1H), 7.81 (m, 1H), 7.74-7.69 (m, 3H), 7.17 (d, J = 1.8 Hz, 1H), |
| | | | 7.11 (m, 1H), 5.08 (m, 1H), 4.58 (s, 2H), 4.40-4.33 (m, 2H), |
| | | | 4.31-4.24 (m, 2H), 3.99 (m, 2H), 3.81-3.72 (m, 1H), 3.16 (br |
| | | | d, J = 7.3 Hz, 3H), 3.07-3.01 (m, 1H), 2.94-2.85 (m, 2H), 2.80- |
| | | | 2.70 (m, 1H), 2.64-2.55 (m, 2H), 2.41-2.26 (m, 3H), 2.08 (s, |
| | | | 1H), 2.03-1.94 (m, 2H), 1.90 (br d, J = 11.6 Hz, 1H), 1.72-1.64 |
| | | | (m, 2H), 1.60 (br d, J = 12.8 Hz, 2H), 1.44 (br d, J = 10.6 Hz, |
| | | | 2H), 1.33 (br d, J = 8.0 Hz, 2H) |
| 69 | 874 | 875 | δ11.01 (s, 1H), 8.46 (s, 1H), 8.13-8.08 (m, 1H), 8.00 (br s, 1H), |
| | | | 7.83-7.79 (m, 1H), 7.77-7.73 (m, 1H), 7.71 (d, J = 7.3 Hz, 2H), |
| | | | 7.61 (br t, J = 7.7 Hz, 2H), 7.52-7.46 (m, 3H), 7.41 (s, 1H), 7.35 |
| | | | (d, J = 7.4 Hz, 1H), 7.14 (d, J = 8.1 Hz, 1H), 5.17-5.11 (m, 1H), |
| | | | 4.63 (d, J = 1.4 Hz, 2H), 4.51-4.43 (m, 1H), 4.38 (s, 2H), 4.31- |
| | | | 4.24 (m, 1H), 4.02-3.95 (m, 2H), 3.88-3.81 (m, 1H), 3.14 (br |
| | | | s, 2H), 3.08-3.01 (m, 1H), 2.94 (br s, 2H), 2.79-2.71 (m, 1H), |
| | | | 2.64-2.56 (m, 2H), 2.47-2.39 (m, 1H), 2.32-2.26 (m, 2H), 2.04- |
| | | | 1.99 (m, 1H), 1.98-1.87 (m, 2H), 1.65-1.57 (m, 2H), 1.52- |
| | | | 1.40 (m, 7H), 1.27 (br d, J = 7.1 Hz, 2H) |
| 70 | 814 | 815 | δ 10.98 (s, 1H), 8.47 (br s, 1H), 8.04 (br s, 1H), 7.85-7.78 (m, |
| | | | 2H), 7.70 (br d, J = 7.5 Hz, 2H), 7.67-7.58 (m, 3H), 7.53-7.45 |
| | | | (m, 3H), 7.43-7.32 (m, 2H), 5.10 (dd, J = 5.0, 13.3 Hz, 1H), 4.47- |
| | | | 4.23 (m, 2H), 4.10-3.69 (m, 3H), 3.24-3.11 (m, 2H), 3.08- |
| | | | 2.66 (m, 8H), 2.65-2.56 (m, 2H), 2.43-2.32 (m, 1H), 2.26-2.16 |
| | | | (m, 1H), 2.11-1.92 (m, 5H), 1.82-1.53 (m, 8H), 1.50-1.31 (m, |
| | | | 3H) |
| 71 | 814 | 815 | δ 11.04 (s, 1H), 8.51 (s, 1H), 8.25 (s, 1H), 8.06 (br s, 1H), 7.87 |
| | | | (d, J = 7.8 Hz, 1H), 7.83-7.75 (m, 3H), 7.73-7.64 (m, 3H), 7.60- |
| | | | 7.52 (m, 3H), 7.50-7.42 (m, 2H), 5.24-5.08 (m, 1H), 4.55- |
| | | | 4.44 (m, 1H), 4.41-4.28 (m, 2H), 4.16-3.98 (m, 2H), 3.23-3.14 |
| | | | (m, 2H), 3.12-3.00 (m, 4H), 2.99-2.90 (m, 3H), 2.86-2.72 (m, |
| | | | 2H), 2.70-2.61 (m, 1H), 2.50-2.42 (m, 1H), 2.40-2.28 (m, 3H), |
| | | | 2.10-1.93 (m, 5H), 1.77 (br s, 5H), 1.63-1.48 (m, 3H), 1.44- |
| | | | 1.32 (m, 1H) |
| 72 | 829 | 830 | δ 11.08 (s, 1H), 8.45 (s, 1H), 8.20 (s, 1H), 8.00 (br s, 1H), 7.81 |
| | | | (d, J = 7.6 Hz, 1H), 7.78-7.69 (m, 3H), 7.65-7.58 (m, 2H), 7.50 |
| | | | (br t, J = 7.5 Hz, 2H), 7.44-7.35 (m, 1H), 7.09 (s, 1H), 7.03- |
| | | | 6.94 (m, 1H), 6.93-6.82 (m, 1H), 5.33 (dd, J = 5.3, 12.7 Hz, 1H), |
| | | | 4.32-4.22 (m, 1H), 4.13-3.92 (m, 3H), 3.28-3.22 (m, 3H), 3.11 |
| | | | (br s, 2H), 3.05-2.95 (m, 4H), 2.92-2.82 (m, 3H), 2.80-2.57 |
| | | | (m, 3H), 2.31-2.21 (m, 3H), 1.98 (br d, J = 10.0 Hz, 3H), 1.94 |
| | | | (br d, J = 7.5 Hz, 2H), 1.75 (br s, 4H), 1.68 (br d, J = 12.1 Hz, |
| | | | 2H), 1.58-1.41 (m, 3H) |
| 73 | 843 | 844 | δ11.14 (s, 1H), 8.53 (s, 1H), 8.22 (s, 1H), 8.08 (br s, 1H), 7.87 (br |
| | | | d, J = 7.9 Hz, 1H), 7.80-7.74 (m, 2H), 7.73-7.63 (m, 2H), 7.56 |
| | | | (t, J = 7.6 Hz, 3H), 7.49-7.41 (m, 1H), 7.16-7.01 (m, 2H), 7.00- |
| | | | 6.90 (m, 1H), 5.39 (dd, J = 5.4, 12.8 Hz, 1H), 4.03-3.74 (m, |
| | | | 3H), 3.18-3.05 (m, 3H), 3.04-2.89 (m, 3H), 2.88-2.79 (m, 3H), |
| | | | 2.79-2.63 (m, 3H), 2.21 (br d, J = 5.9 Hz, 1H), 2.12-1.87 (m, |
| | | | 7H), 1.84-1.64 (m, 8H), 1.63-1.39 (m, 4H), 1.25-0.99 (m, 3H) |
| 74 | 788 | 789 | δ 10.76 (s, 1H), 8.45 (br s, 1H), 8.14 (s, 1H), 7.99 (br s, 1H), 7.81 |
| | | | (br d, J = 7.6 Hz, 1H), 7.77-7.66 (m, 3H), 7.65-7.55 (m, 2H), |
| | | | 7.50 (br t, J = 7.6 Hz, 2H), 7.44-7.37 (m, 1H), 6.96 (br d, J = 8.4 |
| | | | Hz, 2H), 6.62 (br d, J = 8.5 Hz, 2H), 5.74-5.63 (m, 1H), 4.38- |
| | | | 4.19 (m, 3H), 4.06-3.85 (m, 4H), 3.07 (br dd, J = 1.3, 5.8 Hz, |
| | | | 3H), 2.75 (br s, 9H), 2.30 (br dd, J = 2.5, 17.0 Hz, 2H), 2.14- |
| | | | 2.05 (m, 1H), 2.03-1.86 (m, 3H), 1.85-1.73 (m, 3H), 1.72-1.54 |
| | | | (m, 6H), 1.49-1.29 (m, 2H) |
| 75 | 829 | 830 | δ 11.08 (br s, 1H), 8.53-8.44 (m, 1H), 8.26 (s, 1H), 8.04 (br d, |
| | | | J = 1.1 Hz, 1H), 7.85-7.80 (m, 1H), 7.75-7.68 (m, 2H), 7.67- |
| | | | 7.58 (m, 2H), 7.50 (q, J = 7.1 Hz, 3H), 7.44-7.37 (m, 1H), 7.13- |
| | | | 7.02 (m, 1H), 7.02-6.96 (m, 1H), 6.94-6.83 (m, 1H), 5.33 (br |
| | | | dd, J = 5.5, 12.9 Hz, 1H), 4.12-3.68 (m, 4H), 3.22-3.09 (m, |
| | | | 2H), 3.08-2.80 (m, 7H), 2.77-2.57 (m, 5H), 2.27-2.14 (m, 1H), |
| | | | 2.07 (br s, 1H), 2.03-1.95 (m, 3H), 1.76-1.56 (m, 9H), 1.50- |
| | | | 1.31 (m, 4H) |
| 76 | 829 | 830 | δ 8.24 (s, 1H), 7.93 (br s, 1H), 7.72 (d, J = 8.4 Hz, 1H), 7.70- |
| | | | 7.65 (m, 1H), 7.62 (br d, J = 7.9 Hz, 1H), 7.56 (br d, J = 7.5 Hz, |
| | | | 2H), 7.51-7.45 (m, 1H), 7.39 (br t, J = 7.6 Hz, 2H), 7.33-7.28 |
| | | | (m, 1H), 6.93 (br d, J = 8.4 Hz, 1H), 6.87 (br s, 1H), 5.13 (br dd, |
| | | | J = 5.2, 13.2 Hz, 2H), 4.53 (br d, J = 13.6 Hz, 1H), 4.39-4.30 |
| | | | (m, 2H), 4.24-4.18 (m, 1H), 3.87-3.76 (m, 2H), 2.92-2.80 (m, |
| | | | 2H), 2.79-2.70 (m, 1H), 2.60 (br s, 3H), 2.50-2.41 (m, 1H), |
| | | | 2.29-2.20 (m, 2H), 2.17-2.02 (m, 6H), 1.92 (br d, J = 2.1 Hz, |
| | | | 2H), 1.84 (br d, J = 12.0 Hz, 2H), 1.75-1.74 (m, 1H), 1.74 (br s, |
| | | | 1H), 1.57 (br s, 1H), 1.51-1.31 (m, 4H), 1.23-1.15 (m, 1H), |
| | | | 0.97 (br d, J = 9.1 Hz, 2H) |
| 77 | 843 | 844 | δ 11.00 (s, 1H), 8.45 (br s, 1H), 8.02 (br s, 1H), 7.83 (br d, J = 7.4 |
| | | | Hz, 1H), 7.74 (br d, J = 7.6 Hz, 3H), 7.68-7.61 (m, 2H), 7.53 (br |
| | | | t, J = 7.6 Hz, 3H), 7.46-7.41 (m, 1H), 7.27 (br s, 1H), 7.13 (br |
| | | | d, J = 8.5 Hz, 1H), 5.11 (dd, J = 13.3, 5.1 Hz, 1H), 4.79 (br s, 1H), |
| | | | 4.46-4.36 (m, 2H), 4.33-4.26 (m, 1H), 3.86 (br d, J = 2.5 Hz, |
| | | | 4H), 3.32-3.23 (m, 1H), 3.18-3.06 (m, 1H), 3.00-2.89 (m, 2H), |
| | | | 2.78 (br dd, J = 6.4, 3.1 Hz, 1H), 2.63 (br d, J = 16.3 Hz, 2H), |
| | | | 2.48-2.35 (m, 1H), 2.07-1.91 (m, 5H), 1.79 (br d, J = 11.6 Hz, |
| | | | 1H), 1.64 (br d, J = 0.8 Hz, 4H), 1.57-1.47 (m, 2H), 1.42 (br d, |
| | | | J = 13.9 Hz, 2H), 1.37-1.21 (m, 4H) |
| 78 | 828 | 829 | δ 10.88 (s, 1H), 8.38 (s, 1H), 7.94 (br s, 1H), 7.78-7.47 (m, 7H), |
| | | | 7.45-7.36 (m, 3H), 7.34-7.26 (m, 2H), 5.01 (dd, J = 5.1, 13.2 |
| | | | Hz, 1H), 4.39-4.14 (m, 3H), 4.06-3.77 (m, 3H), 3.08-2.88 (m, |
| | | | 3H), 2.87-2.70 (m, 3H), 2.62-2.45 (m, 5H), 2.34-2.19 (m, 2H), |
| | | | 2.18-1.82 (m, 7H), 1.75-1.46 (m, 8H), 1.43-1.19 (m, 3H) |
| 79 | 843 | 844 | δ 10.98 (s, 1H), 8.44-8.40 (m, 1H), 8.06-7.96 (m, 1H), 7.81 (br |
| | | | d, J = 7.6 Hz, 1H), 7.71 (br d, J = 7.4 Hz, 3H), 7.63-7.58 (m, |
| | | | 2H), 7.50 (br t, J = 7.6 Hz, 3H) 7.43-7.37 (m, 1H), 7.18 (br d, |
| | | | J = 4.3 Hz, 1H), 7.05 (br dd, J = 8.2, 1.9 Hz, 1 H), 5.08 (dd, J = |
| | | | 13.2, 5.1 Hz, 1H), 4.53-4.44 (m, 1H), 4.41-4.32 (m, 2H), 4.29- |
| | | | 4.23 (m, 1H), 3.81 (br d, J = 2.5 Hz, 2H), 2.96-2.86 (m, 1H), |
| | | | 2.71-2.55 (m, 4H), 2.46-2.25 (m, 4H), 2.18 (br d, J = 9.2 Hz, |
| | | | 2H), 2.02-1.85 (m, 6H), 1.79-1.73 (m, 1H), 1.63 (br s, 4H), |
| | | | 1.46-1.23 (m, 8H), 1.07-0.81 (m, 2H) |
| 80 | 844 | 845 | δ 11.23-10.43 (m, 1H), 8.42 (br s, 1H), 8.07-7.94 (m, 1H), 7.81 |
| | | | (br d, J = 7.8 Hz, 1H), 7.72 (d, J = 7.4 Hz, 3H), 7.62 (d, J = 8.4 |
| | | | Hz, 2H), 7.51 (t, J = 7.6 Hz, 3H), 7.43-7.38 (m, 1H), 7.18 (s, |
| | | | 1H), 7.05 (dd, J = 8.4, 1.9 Hz, 1H), 5.08 (dd, J = 13.3, 5.1 Hz, |
| | | | 1H), 4.48 (br s, 1H), 4.42-4.35 (m, 1H), 4.31-4.21 (m, 1H), |
| | | | 3.81 (br d, J = 1.9 Hz, 1H), 3.53-3.41 (m, 3H), 2.97-2.84 (m, |
| | | | 1H), 2.77-2.65 (m, 3H), 2.62-2.56 (m, 1H), 2.46-2.39 (m, 3H), |
| | | | 2.38-2.31 (m, 4H), 2.23 (br s, 2H), 2.12 (br d, J = 5.3 Hz, 1H), |
| | | | 2.02-1.83 (m, 6H), 1.80-1.72 (m, 1H), 1.61 (br d, J = 2.0 Hz, |
| | | | 3H), 1.49-1.35 (m, 2H), 1.34-1.17 (m, 3H) |
| 81 | 857 | 858 | δ 10.98 (s, 1H), 8.42 (br s, 1H), 8.05-7.94 (m, 1H), 7.81 (br d, |
| | | | J = 7.6 Hz, 1H), 7.71 (br d, J = 7.5 Hz, 3H), 7.66-7.57 (m, 2H), |
| | | | 7.50 (br t, J = 7.5 Hz, 3H), 7.44-7.37 (m, 1H), 7.27-7.18 (m, |
| | | | 1H), 7.10 (br d, J = 7.2 Hz, 1H), 5.08 (dd, J = 13.4, 4.9 Hz, 1H), |
| | | | 4.81-4.65 (m, 1H), 4.43-4.20 (m, 3H), 4.02-3.61 (m, 5H), 3.07- |
| | | | 2.83 (m, 2H), 2.81-2.65 (m, 2H), 2.43-2.21 (m, 3H), 2.05- |
| | | | 0.81 (m, 20H) |
| 82 | 858 | 859 | δ 10.97 (s, 1H), 8.45-8.39 (m, 1H), 7.99 (br s, 1H), 7.83-7.78 |
| | | | (m, 1H), 7.71 (br d, J = 7.4 Hz, 3H), 7.66-7.57 (m, 2H), 7.50 (br |
| | | | t, J = 7.6 Hz, 3H), 7.44-7.38 (m, 1H), 7.23 (s, 1H), 7.10 (br d, |
| | | | J = 8.4 Hz, 1H), 5.13-5.04 (m, 1H), 4.77 (br s, 1H), 4.43-4.35 |
| | | | (m, 1H), 4.31-4.23 (m, 1H), 3.94-3.63 (m, 4H), 3.59-3.45 (m, |
| | | | 2H), 3.29-3.11 (m, 3H), 3.01-2.83 (m, 2H), 2.82-2.65 (m, 2H), |
| | | | 2.60 (br d, J = 17.5 Hz, 2H), 2.47-2.27 (m, 3H), 2.10-1.83 (m, |
| | | | 6H), 1.77 (br d, J = 13.0 Hz, 1H), 1.71-1.50 (m, 3H), 1.47-1.33 |
| | | | (m, 2H), 1.30-1.17 (m, 2H) |
| 83 | 858 | 859 | δ 10.98 (s, 1H), 9.47 (br s, 1H), 8.47 (s, 1H), 8.00 (br s, 1H), 7.82 |
| | | | (td, J = 1.4, 7.8 Hz, 1H), 7.77-7.69 (m, 3H), 7.68-7.58 (m, 3H), |
| | | | 7.51 (t, J = 7.6 Hz, 2H), 7.45-7.38 (m, 1H), 7.25 (s, 1H), 7.12 |
| | | | (br d, J = 9.1 Hz, 1H), 5.09 (dd, J = 5.0, 13.3 Hz, 1H), 4.82 (br s, |
| | | | 1H), 4.46-4.23 (m, 5H), 4.08-3.85 (m, 4H), 3.43-3.29 (m, 3H), |
| | | | 3.25-3.15 (m, 1H), 3.11-2.87 (m, 4H), 2.80 (br t, J = 11.4 Hz, |
| | | | 1H), 2.66-2.56 (m, 1H), 2.47-2.31 (m, 1H), 2.09-1.81 (m, 9H), |
| | | | 1.78-1.67 (m, 1H), 1.61 (br d, J = 8.1 Hz, 1H), 1.52-1.30 (m, |
| | | | 2H) |
| 84 | 789 | 790 | δ 10.76 (s, 1H), 8.46 (s, 1H), 8.15 (s, 1H), 8.00 (br s, 1H), 7.81 |
| | | | (d, J = 7.8 Hz, 1H), 7.71 (br d, J = 7.4 Hz, 3H), 7.61 (br t, J = 7.6 |
| | | | Hz, 2H), 7.51 (t, J = 7.6 Hz, 2H), 7.44-7.37 (m, 1H), 6.76 (d, |
| | | | J = 8.9 Hz, 2H), 6.60 (d, J = 8.9 Hz, 2H), 5.38 (br d, J = 3.8 Hz, |
| | | | 1H), 4.33-4.24 (m, 1H), 4.23-4.15 (m, 1H), 4.02-3.93 (m, 1H), |
| | | | 3.88 (br d, J = 13.0 Hz, 1H), 3.43 (br d, J = 11.8 Hz, 2H), 3.19- |
| | | | 3.06 (m, 2H), 2.81-2.68 (m, 3H), 2.66-2.57 (m, 7H), 2.55 (br |
| | | | s, 4H), 2.40-2.32 (m, 1H), 2.10 (qd, J = 13.1, 4.4 Hz, 1H), 1.94 |
| | | | (br dd, J = 14.3, 3.2 Hz, 2H), 1.89-1.78 (m, 4H), 1.60-1.41 (m, |
| | | | 3H), 1.34 (br d, J = 9.9 Hz, 1H), 1.23 (d, J = 6.5 Hz, 1H) |
| 85 | 844 | 845 | δ10.96 (s, 1H), 8.45 (s, 1H), 8.17 (s, 1H), 8.00 (br s, 1H), 7.81 |
| | | | (dd, J = 1.6, 7.7 Hz, 1H), 7.76-7.69 (m, 3H), 7.66-7.57 (m, 3H), |
| | | | 7.50 (t, J = 7.6 Hz, 2H), 7.42 (d, J = 7.3 Hz, 1H), 7.18 (d, J = 1.5 |
| | | | Hz, 1H), 7.05 (dd, J = 1.9, 8.4 Hz, 1H), 5.12-5.01 (m, 1H), 4.55- |
| | | | 4.45 (m, 1H), 4.42-4.34 (m, 1H), 4.31-4.19 (m, 2H), 4.13- |
| | | | 3.91 (m, 3H), 3.05-2.73 (m, 7H), 2.71-2.56 (m, 3H), 2.45-2.31 |
| | | | (m, 1H), 2.25-2.12 (m, 4H), 2.03-1.86 (m, 6H), 1.66 (br d, J = |
| | | | 6.8 Hz, 4H), 1.55-1.40 (m, 2H), 1.38-1.23 (m, 1H), 1.19-1.03 |
| | | | (m, 2H) |
| 86 | 828 | 829 | δ 11.15-10.90 (m, 1H), 9.29-9.25 (m, 1H), 8.59 (br d, J = 6.3 |
| | | | Hz, 1H), 8.50-8.42 (m, 1H), 8.39-8.32 (m, 1H), 8.23-8.19 (m, |
| | | | 1H), 8.05-7.96 (m, 1H), 7.81 (br d, J = 7.6 Hz, 1H), 7.73-7.69 |
| | | | (m, 2H), 7.65-7.59 (m, 2H), 7.53-7.48 (m, 2H), 7.43-7.38 (m, |
| | | | 1H), 5.10 (dd, J = 5.1, 13.2 Hz, 1H), 4.49-4.20 (m, 3H), 4.06- |
| | | | 3.82 (m, 3H), 3.19-3.07 (m, 3H), 3.01-2.85 (m, 5H), 2.83-2.71 |
| | | | (m, 2H), 2.67-2.54 (m, 4H), 2.46-2.31 (m, 2H), 2.29-2.09 (m, |
| | | | 4H), 2.04-1.84 (m, 5H), 1.81-1.55 (m, 5H), 1.52-1.36 (m, 3H), |
| | | | 1.34-1.18 (m, 1H) |
| 87 | 830 | 831 | δ 10.97 (s, 1H), 8.45 (s, 1H), 8.16 (s, 1H), 8.00 (br s, 1H), 7.81 |
| | | | (br d, J = 7.8 Hz, 1H), 7.75-7.68 (m, 3H), 7.65-7.57 (m, 3H), |
| | | | 7.50 (t, J = 7.6 Hz, 2H), 7.43-7.37 (m, 1H), 7.18 (s, 1H), 7.05 |
| | | | (dd, J = 1.9, 8.4 Hz, 1H), 5.07 (dd, J = 5.1, 13.3 Hz, 1H), 4.50 (br |
| | | | dd, J = 3.9, 7.4 Hz, 1H), 4.42-4.33 (m, 1H), 4.29-4.20 (m, 2 |
| | | | H), 4.09-3.92 (m, 3H), 3.16-2.97 (m, 4H), 2.95-2.70 (m, 6H), |
| | | | 2.48-2.38 (m, 2H), 2.29 (br s, 1H), 2.07-1.87 (m, 7H), 1.74 (br |
| | | | d, J = 10.4 Hz, 2H), 1.64 (br d, J = 8.3 Hz, 2H), 1.49-1.40 (m, |
| | | | 4H) |
| 88 | 828 | 829 | δ 10.98 (s, 1H), 8.45 (s, 1H), 8.17 (s, 1H), 8.00 (br s, 1H), 7.81 |
| | | | (td, J = 1.3, 8.0 Hz, 1H), 7.77-7.55 (m, 6H), 7.53-7.46 (m, 3H), |
| | | | 7.44-7.37 (m, 2H), 5.10 (dd, J = 5.1, 13.3 Hz, 1H), 4.48-4.37 |
| | | | (m, 1H), 4.32-4.25 (m, 2H), 4.02-3.91 (m, 2H), 3.12-3.07 (m, |
| | | | 2H), 3.05-2.99 (m, 2H), 2.96-2.85 (m, 2H), 2.65-2.58 (m, 5H), |
| | | | 2.41-2.32 (m, 1H), 2.28-2.16 (m, 4H), 2.04-1.86 (m, 4H), 1.85- |
| | | | 1.70 (m, 4H), 1.70-1.52 (m, 5H), 1.50-1.24 (m, 3H) |
| 89 | 844 | 845 | δ 10.95 (s, 1H), 8.44 (s, 1H), 8.16 (s, 1H), 7.99 (br s, 1H), 7.80 |
| | | | (d, J = 7.8 Hz, 1H), 7.76-7.66 (m, 3H), 7.64-7.53 (m, 3H), 7.49 |
| | | | (t, J = 7.6 Hz, 2H), 7.43-7.36 (m, 1H), 7.18 (d, J = 1.3 Hz, 1H), |
| | | | 7.05 (dd, J = 1.9, 8.4 Hz, 1H), 5.06 (dd, J = 5.0, 13.3 Hz, 1H), |
| | | | 4.57-4.45 (m, 1H), 4.41-4.20 (m, 3H), 4.05-3.85 (m, 3H), 3.21- |
| | | | 3.04 (m, 2H), 2.99-2.84 (m, 3H), 2.82-2.66 (m, 3H), 2.59 (br |
| | | | d, J = 15.6 Hz, 2H), 2.43-2.23 (m, 4H), 2.18-1.83 (m, 8H), 1.72- |
| | | | 1.51 (m, 6H), 1.49-1.22 (m, 2H) |
| 90 | 844 | 845 | δ 11.03 (s, 1H), 8.51 (br s, 1H), 8.24 (s, 1H), 8.05 (br s, 1H), 7.86 |
| | | | (d, J = 7.8 Hz, 1H), 7.82-7.73 (m, 3H), 7.71-7.62 (m, 3H), 7.56 |
| | | | (t, J = 7.6 Hz, 2H), 7.49-7.43 (m, 1H), 7.23 (s, 1H), 7.11 (dd, |
| | | | J = 1.8, 8.4 Hz, 1H), 5.13 (dd, J = 5.1, 13.3 Hz, 1H), 4.55 (td, J = |
| | | | 4.1, 7.6 Hz, 1H), 4.48-4.26 (m, 4H), 4.09-3.99 (m, 2H), 3.19 |
| | | | (br t, J = 11.8 Hz, 1H), 3.09-2.95 (m, 3H), 2.93-2.79 (m, 3H), |
| | | | 2.65 (br d, J = 7.1 Hz, 3H), 2.53-2.44 (m, 3H), 2.43-2.31 (m, |
| | | | 2H), 2.15-1.92 (m, 7H), 1.84-1.63 (m, 4H), 1.60-1.35 (m, 4H) |
| 275 | 847 | 848 | δ (DMSO-d6) 11.11 (s, 1H), 8.48 (s, 1H), 7.98 (br s, 1H), 7.83- |
| | | | 7.74 (m, 4H), 7.64-7.58 (m, 1H), 7.34 (t, J = 8.8 Hz, 2H), 7.08 |
| | | | (d, J = 8.1 Hz, 1H), 7.03 (s, 1H), 6.91 (br d, J = 8.0 Hz, 1H), 5.42- |
| | | | 5.31 (m, 1H), 4.35-4.18 (m, 2H), 4.09-3.98 (m, 2H), 3.76- |
| | | | 3.61 (m, 5H), 3.35 (s, 3H), 3.24-3.12 (m, 4H), 2.98-2.86 (m, |
| | | | 4H), 2.73-2.58 (m, 3H), 2.22-2.10 (m, 1H), 2.09-1.90 (m, 9H), |
| | | | 1.85-1.62 (m, 2H), 1.58-1.49 (m, 1H), 1.45-1.33 (m, 1H) |
| 276 | 875 | 876 | δ (DMSO-d6) 11.09 (s, 1H), 8.48-8.39 (m, 1H), 8.15 (s, 1H), |
| | | | 8.04-7.93 (m, 1H), 7.82-7.73 (m, 4H), 7.65-7.57 (m, 1H), 7.45 |
| | | | (br d, J = 7.4 Hz, 2H), 7.33 (t, J = 8.8 Hz, 2H), 7.12-7.07 (m, |
| | | | 1H), 7.04-6.96 (m, 1H), 6.91 (d, J = 8.3 Hz, 1H), 5.39-5.29 (m, |
| | | | 1H), 3.74-3.64 (m, 1H), 3.63-3.52 (m, 1H), 2.96 (br d, J = 9.1 |
| | | | Hz, 2H), 2.92-2.85 (m, 3H), 2.83-2.76 (m, 2H), 2.73-2.58 (m, |
| | | | 2H), 2.20 (br s, 2H), 2.11-1.92 (m, 8H), 1.85-1.63 (m, 10H), |
| | | | 1.57-1.45 (m, 1H), 1.44-1.29 (m, 5H), 1.24-1.14 (m, 2H) |
| 277 | 845 | 846 | δ 10.89 (s, 1H), 9.45 (br s, 1H), 8.47 (s, 1H), 7.97 (br s, 1H), 7.82- |
| | | | 7.66 (m, 6H), 7.63-7.58 (m, 1H), 7.41 (s, 1H), 7.33 (t, J = 8.9 |
| | | | Hz, 2H), 7.07-6.96 (m, 1H), 4.42-4.23 (m, 4H), 4.08-4.01 (m, |
| | | | 1H), 4.00-3.96 (m, 3H), 3.74-3.61 (m, 3H), 3.27-3.17 (m, 2H), |
| | | | 3.08 (br s, 3H), 2.98 (br d, J = 8.8 Hz, 3H), 2.75-2.58 (m, 2H), |
| | | | 2.45-2.24 (m, 2H), 2.21-2.11 (m, 2H), 2.09-1.91 (m, 8H), 1.80- |
| | | | 1.23 (m, 6H) |
| 278 | 875 | 876 | δ 10.89 (s, 1H), 9.49 (br d, J = 1.9 Hz, 1H), 8.48 (s, 1H), 7.98 (br |
| | | | s, 1H), 7.88-7.56 (m, 6H), 7.53-7.15 (m, 3H), 7.13-6.94 (m, |
| | | | 1H), 6.23-5.98 (m, 1H), 5.00-4.85 (m, 1H), 4.81-4.57 (m, 2H), |
| | | | 4.53-4.39 (m, 2H), 4.34 (br dd, J = 5.1, 9.6 Hz, 2H), 4.27 (br d, |
| | | | J = 7.8 Hz, 1H), 4.13-4.02 (m, 2H), 3.98 (s, 3H), 3.83-3.59 (m, |
| | | | 2H), 3.57-3.46 (m, 1H), 3.35-3.07 (m, 4H), 2.97 (br t, J = 10.5 |
| | | | Hz, 2H), 2.77-2.59 (m, 3H), 2.45-2.24 (m, 3H), 2.23-2.11 (m, |
| | | | 1H), 2.04-1.92 (m, 3H), 1.88 (br d, J = 11.3 Hz, 2H), 1.74-1.61 |
| | | | (m, 1H), 1.59-1.47 (m, 1H), 1.46-1.18 (m, 1H) |
| 279 | 860 | 861 | δ (DMSO-d6) 11.12 (s, 1H), 8.44 (br s, 1H), 8.18 (s, 1H), 8.06- |
| | | | 7.94 (m, 1H), 7.82-7.71 (m, 4H), 7.65-7.52 (m, 2H), 7.34 (br t, |
| | | | J = 8.6 Hz, 2H), 7.10 (br s, 1H), 7.03 (br d, J = 8.1 Hz, 1H), 6.95- |
| | | | 6.86 (m, 1H), 5.36 (br dd, J = 12.7, 5.3 Hz, 1H), 4.43-4.30 (m, |
| | | | 1H), 4.12-3.93 (m, 1H), 3.90-3.73 (m, 2H), 3.56-3.36 (m, 3H), |
| | | | 3.22-3.11 (m, 2H), 3.09-2.99 (m, 1H), 2.98-2.81 (m, 2H), 2.78- |
| | | | 2.56 (m, 6H), 2.50-2.39 (m, 1H), 2.37-2.06 (m, 2H), 2.05- |
| | | | 1.89 (m, 3H), 1.88-1.70 (m, 6H), 1.69-1.55 (m, 2H), 1.44 (br |
| | | | dd, J = 7.5, 1.7 Hz, 4H), 1.29 (br dd, J = 11.7, 1.8 Hz, 2H), 0.94 |
| | | | (br s, 1H) |
| 280 | 842 | 843 | δ (DMSO-d6) 11.09 (s, 1H), 8.47-8.39 (m, 1H), 8.15 (s, 1H), |
| | | | 8.06-7.95 (m, 1H), 7.81 (br d, J = 6.6 Hz, 1H), 7.76-7.67 (m, |
| | | | 3H), 7.66-7.58 (m, 1H), 7.51 (br t, J = 7.3 Hz, 3H), 7.45-7.37 |
| | | | (m, 1H), 7.11-6.99 (m, 2H), 6.95-6.87 (m, 1H), 5.39-5.30 (m, |
| | | | 1H), 4.41-4.29 (m, 1H), 3.98-3.74 (m, 2H), 3.34 (s, 3H), 3.21- |
| | | | 3.10 (m, 2H), 2.97-2.83 (m, 2H), 2.76-2.57 (m, 6H), 2.32- |
| | | | 2.16 (m, 2H), 2.01-1.93 (m, 2H), 1.88-1.65 (m, 8H), 1.63-1.54 |
| | | | (m, 2H), 1.50-1.37 (m, 4H), 1.33-1.22 (m, 3H), 1.08-0.82 (m, |
| | | | 2H) |
| 281 | 842 | 843 | δ (DMSO-d6) 11.10 (s, 1H), 8.42 (br s, 1H), 8.16 (s, 1H), 7.99 |
| | | | (br s, 1H), 7.81 (br d, J = 7.3 Hz, 1H), 7.72 (br d, J = 7.5 Hz, 3H), |
| | | | 7.65-7.58 (m, 1H), 7.51 (br t, J = 7.3 Hz, 3H), 7.44-7.38 (m, |
| | | | 1H), 7.11-6.99 (m, 2H), 6.91 (br d, J = 6.9 Hz, 1H), 5.39-5.31 |
| | | | (m, 1H), 4.36 (br d, J = 4.8 Hz, 1H), 3.93-3.74 (m, 2H), 3.34 (s, |
| | | | 3H), 3.16 (br s, 2H), 2.97-2.84 (m, 2H), 2.78-2.57 (m, 6H), |
| | | | 2.35-2.20 (m, 2H), 2.04-1.91 (m, 3H), 1.88-1.69 (m, 7H), 1.64- |
| | | | 1.54 (m, 2H), 1.49-1.38 (m, 4H), 1.34-1.22 (m, 3H), 1.00- |
| | | | 0.78 (m, 2H) |
| 282 | 839 | 840 | δ (DMSO-d6) 10.82 (br s, 1H), 8.46 (br s, 1H), 7.98 (br s, 1H), |
| | | | 7.81-7.74 (m, 4H), 7.61 (br t, J = 7.4 Hz, 2H), 7.33 (br t, J = 8.3 |
| | | | Hz, 2H), 7.03-6.98 (m, 2H), 6.96-6.91 (m, 1H), 5.01 (s, 1H), |
| | | | 4.48-4.41 (m, 1H), 4.34-4.24 (m, 1H), 4.09-3.95 (m, 4H), 3.80 |
| | | | (br dd, J = 11.6, 4.4 Hz, 1H), 3.24-3.14 (m, 2H), 3.11-2.97 (m, |
| | | | 6H), 2.84-2.72 (m, 2H), 2.63 (br dd, J = 11.6, 5.3 Hz, 2H), 2.24- |
| | | | 2.15 (m, 1H), 2.00 (br dd, J = 12.8, 4.1 Hz, 2H), 1.92-1.88 (m, |
| | | | 1H), 1.80-1.71 (m, 3H), 1.69-1.63 (m, 4H), 1.45-1.32 (m, 3H) |
| 283 | 781 | 782 | δ (DMSO-d6) 10.82 (s, 1H), 8.43 (br s, 1H), 7.96 (br s, 1H), 7.82- |
| | | | 7.67 (m, 4H), 7.66-7.47 (m, 2H), 7.32 (br d, J = 7.6 Hz, 2H), |
| | | | 7.06-6.90 (m, 3H), 4.45-4.30 (m, 1H), 4.06-3.72 (m, 3H), 2.98 |
| | | | (br s, 4H), 2.90-2.74 (m, 2H), 2.71-2.57 (m, 4H), 2.41-2.29 |
| | | | (m, 1H), 2.41-2.09 (m, 2H), 2.08-1.83 (m, 4H), 1.82-1.70 (m, |
| | | | 2H), 1.60 (br d, J = 5.0 Hz, 2H), 1.47-1.35 (m, 2H), 1.34-1.05 |
| | | | (m, 5H) |
| 284 | 864 | 865 | δ 10.80 (s, 1H), 8.42 (br s, 1H), 8.14 (s, 0.2H), 8.04-7.90 (m, |
| | | | 1H), 7.81-7.67 (m, 4H), 7.63-7.49 (m, 2H), 7.32 (t, J = 8.8 Hz, |
| | | | 2H), 6.98 (br t, J = 8.4 Hz, 1H), 6.49-6.40 (m, 2H), 6.02 (br d, |
| | | | J = 7.8 Hz, 1H), 4.37-4.25 (m, 1H), 4.20-3.95 (m, 2H), 3.84- |
| | | | 3.61 (m, 5H), 3.54 (br d, J = 1.9 Hz, 2H), 3.05-2.89 (m, 2H), |
| | | | 2.89-2.81 (m, 1H), 2.71 (br dd, J = 4.5, 12.3 Hz, 1H), 2.59 (br s, |
| | | | 2H), 2.39-2.16 (m, 3H), 2.13-2.02 (m, 2H), 2.00-1.82 (m, 4H), |
| | | | 1.81-1.73 (m, 1H), 1.66 (br s, 5H), 1.44-1.13 (m, 5H) |
| 285 | 792 | 793 | δ (CDC13) 10.77 (br s, 1H), 8.44 (br s, 1H), 8.05-7.93 (m, 1H), |
| | | | 7.82-7.72 (m, 5H), 7.61 (br s, 2H), 7.48 (br d, J = 6.8 Hz, 1H), |
| | | | 7.34 (br t, J = 8.8 Hz, 2H), 6.75 (br d, J = 8.9 Hz, 2H), 6.60 (br d, |
| | | | J = 8.9 Hz, 2H), 5.38 (d, J = 7.1 Hz, 1H), 4.23-4.15 (m, 1H), |
| | | | 3.71-3.61 (m, 1H), 3.53-3.47 (m, 1H), 3.43 (br d, J = 11.6 Hz, |
| | | | 2H), 2.90 (br d, J = 10.1 Hz, 2H), 2.78-2.68 (m, 1H), 2.60-2.55 |
| | | | (m, 1H), 2.48 (br s, 1H), 2.31-2.23 (m, 1H), 2.13-2.07 (m, 3H), |
| | | | 2.00-1.91 (m, 3H), 1.77 (br d, J = 8.9 Hz, 5H), 1.63-1.49 (m, |
| | | | 7H), 1.37-1.29 (m, 2H), 1.25 (br s, 1H) |
| 286 | 827 | 828 | δ (DMSO-d6) 11.15 (br d, J = 2.1 Hz, 1H), 8.50 (d, J = 3.7 Hz, |
| | | | 1H), 8.27 (s, 1H), 8.01-7.96 (m, 1H), 7.87 (d, J = 8.3 Hz, 1H), |
| | | | 7.74 (d, J = 7.2 Hz, 2H), 7.71-7.64 (m, 1H), 7.54 (t, J = 7.6 Hz, |
| | | | 2H), 7.44 (t, J = 7.3 Hz, 2H), 7.10 (br d, J = 2.2 Hz, 1H), 7.05- |
| | | | 6.99 (m, 1H), 6.96-6.91 (m, 1H), 5.36 (dd, J = 13.6, 5.6 Hz, 1H), |
| | | | 4.35-4.26 (m, 1H), 3.28 (s, 1H), 3.21-2.74 (m, 9H), 2.73-2.61 |
| | | | (m, 3H), 2.39-2.34 (m, 1H), 2.29-2.11 (m, 3H), 2.09-1.89 (m, |
| | | | 7H), 1.82-1.68 (m, 7H), 1.62-1.43 (m, 3H), 1.30-1.09 (m, 3H) |
| 287 | 861 | 862 | δ (DMSO-d6) 11.10 (s, 1H), 8.46 (br s, 1H), 8.17 (s, 2H), 7.98 |
| | | | (br s, 1H), 7.82-7.74 (m, 4H), 7.68-7.58 (m, 2H), 7.33 (t, J = |
| | | | 8.8 Hz, 2H), 7.10 (s, 1H), 7.01 (d, J = 8.3 Hz, 1H), 6.91 (d, J = |
| | | | 8.3 Hz, 1H), 5.34 (dd, J = 5.4, 12.9 Hz, 1H), 4.32-4.21 (m, 1H), |
| | | | 4.08-3.97 (m, 2H), 3.34 (s, 3H), 3.13-3.05 (m, 2H), 2.97 (br d, |
| | | | J = 9.5 Hz, 2H), 2.93-2.83 (m, 3H), 2.79-2.70 (m, 2H), 2.69- |
| | | | 2.58 (m, 2H), 2.21 (br d, J = 6.5 Hz, 2H), 2.06-1.97 (m, 5H), |
| | | | 1.94-1.87 (m, 2H), 1.78-1.68 (m, 7H), 1.55-1.47 (m, 2H), 1.41- |
| | | | 1.28 (m, 1H), 1.21-1.13 (m, 2H) |
| 288 | 845 | 846 | δ (DMSO-d6) 11.10 (s, 1H), 8.48 (d, J = 3.6 Hz, 1H), 8.22 (s, |
| | | | 1H), 8.15 (s, 1H), 8.01-7.92 (m, 1H), 7.84 (d, J = 8.5 Hz, 1H), |
| | | | 7.76 (dd, J = 8.8, 5.4 Hz, 2H), 7.65 (t, J = 7.8 Hz, 1H), 7.42-7.32 |
| | | | (m, 3H), 7.10 (s, 1H), 7.05-6.96 (m, 1H), 6.92 (br d, J = 8.3 Hz, |
| | | | 1H), 5.39-5.28 (m, 1H), 4.32-4.22 (m, 1H), 4.10-3.88 (m, 2H), |
| | | | 3.23-3.08 (m, 3H), 3.03-2.94 (m, 2H), 2.92-2.75 (m, 4H), 2.66- |
| | | | 2.56 (m, 2H), 2.27-2.17 (m, 2H), 2.10-1.87 (m, 7H), 1.82- |
| | | | 1.63 (m, 7H), 1.61-1.44 (m, 3H), 1.44-1.03 (m, 5H) |
| 289 | 875 | 876 | δ 10.90 (s, 1H), 9.57-9.35 (m, 1H), 8.46 (br s, 1H), 7.97 (br s, |
| | | | 1H), 7.82-7.71 (m, 4H), 7.68-7.57 (m, 3H), 7.41 (s, 1H), 7.33 |
| | | | (t, J = 8.9 Hz, 2H), 7.04 (d, J = 8.5 Hz, 1H), 6.07 (s, 1H), 4.60- |
| | | | 4.51 (m, 1H), 4.46-4.31 (m, 4H), 4.02-3.98 (m, 1H), 3.97 (s, |
| | | | 3H), 3.90-3.72 (m, 2H), 3.40 (br dd, J = 1.7, 9.7 Hz, 2H), 3.27- |
| | | | 3.15 (m, 3H), 2.99 (br s, 1H), 2.87-2.71 (m, 2H), 2.70-2.58 (m, |
| | | | 2H), 2.37-2.25 (m, 3H), 2.22-2.10 (m, 1H), 1.92 (br d, J = 11.1 |
| | | | Hz, 6H), 1.78-1.68 (m, 1H), 1.65-1.54 (m, 1H), 1.51-1.33 (m, |
| | | | 2H), 1.27-1.16 (m, 1H) |
| 290 | 875 | 876 | δ 10.89 (s, 1H), 8.46 (br s, 1H), 8.19-7.97 (br s, 1H), 7.83-7.70 |
| | | | (m, 4H), 7.66-7.57 (m, 3H), 7.43 (s, 1H), 7.33 (t, J = 8.8 Hz, |
| | | | 2H), 7.08-7.00 (m, 1H), 5.83-5.62 (m, 1H), 4.43-4.28 (m, 2H), |
| | | | 4.11-3.96 (m, 5H), 3.47-3.37 (m, 2H), 3.27 (br s, 2H), 3.18- |
| | | | 3.02 (m, 3H), 2.75-2.58 (m, 4H), 2.42-2.28 (m, 2H), 2.25-2.09 |
| | | | (m, 2H), 2.03-1.62 (m, 11H), 1.53-1.13 (m, 3H) |
| 291 | 843 | 844 | δ (DMSO-d6) 11.11 (s, 1H), 8.47 (d, J = 2.6 Hz, 1H), 8.15 (s, |
| | | | 1H), 8.06 (br d, J = 5.6 Hz, 1H), 7.97 (br s, 1H), 7.76-7.67 (m, |
| | | | 2H), 7.64-7.60 (m, 1H), 7.56 (ddd, J = 9.1, 6.8, 2.0 Hz, 1H), |
| | | | 7.31 (br d, J = 7.8 Hz, 1H), 7.09-7.02 (m, 2H), 6.92 (br d, J = |
| | | | 7.0 Hz, 1H), 6.53 (d, J = 9.0 Hz, 1H), 6.37 (br t, J = 6.6 Hz, 1H), |
| | | | 5.36 (br dd, J = 12.8, 5.3 Hz, 1H), 4.43-4.33 (m, 1H), 4.00-3.89 |
| | | | (m, 1H), 3.86-3.76 (m, 1H), 3.28 (br d, J = 7.6 Hz, 3H), 2.99- |
| | | | 2.86 (m, 2H), 2.81-2.58 (m, 7H), 2.47 (br s, 2H), 2.05-1.83 (m, |
| | | | 6H), 1.82-1.71 (m, 3H), 1.70-1.56 (m, 3H), 1.55-1.41 (m, 4H), |
| | | | 1.39 (br s, 4H), 1.12-0.84 (m, 2H) |
| 292 | 855 | 856 | δ (DMSO-d6) 10.96 (s, 1H), 8.45 (br dd, J = 1.5, 2.9 Hz, 1H), |
| | | | 8.06-7.92 (m, 1H), 7.85-7.69 (m, 5H), 7.63 (br d, J = 5.6 Hz, |
| | | | 1H), 7.35 (t, J = 8.7 Hz, 2H), 7.10-7.00 (m, 1H), 6.97-6.89 (m, |
| | | | 1H), 6.80 (br d, J = 7.3 Hz, 1H), 5.20-5.12 (m, 1H), 3.90-3.77 |
| | | | (m, 4H), 3.54-3.45 (m, 5H), 3.05-2.94 (m, 4H), 2.84-2.77 (m, |
| | | | 1H), 2.71-2.61 (m, 2H), 2.22-2.10 (m, 2H), 2.05-1.86 (m, 3H), |
| | | | 1.83-1.74 (m, 3H), 1.74-1.58 (m, 4H), 1.49-1.37 (m, 2H), 1.34- |
| | | | 1.18 (m, 3H) |
| 293 | 862 | 863 | δ (DMSO-d6) 11.22 (br s, 1H), 8.46 (br s, 1H), 7.99 (br s, 1H), |
| | | | 7.82-7.74 (m, 4H), 7.62 (br t, J = 7.7 Hz, 2H), 7.34 (t, J = 8.9 |
| | | | Hz, 2H), 7.21-7.12 (m, 2H), 7.05 (d, J = 7.8 Hz, 1H), 5.38 (dd, |
| | | | J = 12.8, 5.3 Hz, 1H), 4.53 (br d, J = 12.0 Hz, 1H), 4.33-4.20 |
| | | | (m, 2H), 4.03-3.92 (m, 2H), 3.30 (br d, J = 13.8 Hz, 1H), 3.18- |
| | | | 3.08 (m, 3H), 3.03 (br d, J = 13.3 Hz, 1H), 2.88 (br d, J = 12.6 |
| | | | Hz, 3H), 2.74-2.63 (m, 4H), 2.22-2.14 (m, 1H), 2.12-2.04 (m, |
| | | | 2H), 2.00-1.94 (m, 1H), 1.92-1.78 (m, 4H), 1.77-1.68 (m, 1H), |
| | | | 1.66-1.53 (m, 6H), 1.49-1.40 (m, 1H) |
| 294 | 848 | 849 | δ (DMSO-d6) 11.20 (s, 1H), 8.49-8.42 (m, 1H), 8.19-8.15 (m, |
| | | | 1H), 7.98 (br s, 1H), 7.81 (s, 4H), 7.67-7.57 (m, 2H), 7.36-7.29 |
| | | | (m, 2H), 7.17 (d, J = 8.3 Hz, 1H), 7.12-6.98 (m, 1H), 5.39-5.31 |
| | | | (m, 1H), 4.32-4.20 (m, 1H), 4.10-3.93 (m, 2H), 3.35-3.24 (m, |
| | | | 2H), 3.18-3.00 (m, 3H), 2.94 (br d, J = 10.0 Hz, 2H), 2.87-2.76 |
| | | | (m, 3H), 2.75-2.61 (m, 2H), 2.25-2.10 (m, 3H), 2.07-1.86 (m, |
| | | | 6H), 1.78-1.59 (m, 6H), 1.57-1.44 (m, 2H), 1.41-1.27 (m, 1H), |
| | | | 1.22-1.05 (m, 2H) |
| 295 | 830 | 831 | δ (DMSO-d6) 11.24-11.19 (m, 1H), 8.46 (d, J = 2.6 Hz, 1H), |
| | | | 8.11-8.03 (m, 1H), 7.97 (br s, 1H), 7.77-7.66 (m, 2H), 7.64- |
| | | | 7.52 (m, 2H), 7.34-7.26 (m, 2H), 7.22 (d, J = 8.1 Hz, 1H), 7.14- |
| | | | 7.06 (m, 1H), 6.55-6.50 (m, 1H), 6.41-6.33 (m, 1H), 5.41- |
| | | | 5.32 (m, 1H), 4.43-4.31 (m, 1H), 4.00-3.89 (m, 1H), 3.87-3.74 |
| | | | (m, 1H), 3.56-3.37 (m, 2H), 3.06-2.72 (m, 7H), 2.72-2.61 (m, |
| | | | 2H), 2.45 (br d, J = 6.4 Hz, 2H), 2.19-2.11 (m, 1H), 2.04-1.88 |
| | | | (m, 4H), 1.86-1.73 (m, 3H), 1.72-1.65 (m, 1H), 1.63-1.48 (m, |
| | | | 4H), 1.45-1.18 (m, 5H), 1.12-0.85 (m, 2H) |
| 296 | 848 | 849 | δ 11.00 (s, 1H), 8.47 (s, 1H), 7.97 (br s, 1H), 7.84-7.64 (m, 5H), |
| | | | 7.64-7.57 (m, 1H), 7.54-7.45 (m, 1H), 7.40-7.27 (m, 4H), 5.14 |
| | | | (br dd, J = 5.0, 13.1 Hz, 1H), 4.87-4.71 (m, 1H), 4.48-4.34 (m, |
| | | | 3H), 4.30-4.16 (m, 4H), 4.11-3.92 (m, 4H), 3.81-3.60 (m, 3H), |
| | | | 3.17-3.11 (m, 1H), 3.01-2.85 (m, 3H), 2.28-2.06 (m, 4H), 1.98 |
| | | | (br d, J = 4.6 Hz, 6H), 1.84-1.59 (m, 3H), 1.57-1.32 (m, 3H) |
| 297 | 789 | 790 | δ (DMSO-d6) 10.76 (s, 1H), 8.45 (d, J = 3.6 Hz, 1H), 8.17 (s, |
| | | | 1H), 8.04 (br d, J = 8.0 Hz, 1H), 7.98 (s, 1H), 7.80-7.72 (m, 2H), |
| | | | 7.72-7.66 (m, 2H), 7.60 (br d, J = 7.9 Hz, 1H), 7.57-7.51 (m, |
| | | | 1H), 7.30 (br d, J = 8.3 Hz, 1H), 6.74 (br d, J = 8.8 Hz, 2H), 6.59 |
| | | | (br d, J = 8.9 Hz, 2H), 6.51 (d, J = 9.3 Hz, 1H), 6.35 (t, J = 6.6 |
| | | | Hz, 1H), 5.38 (br d, J = 7.0 Hz, 1H), 4.24-4.12 (m, 1H), 3.78- |
| | | | 3.66 (m, 1H), 3.43 (br d, J = 11.8 Hz, 2H), 2.99-2.94 (m, 2H), |
| | | | 2.91 (br t, J = 5.6 Hz, 2H), 2.77-2.67 (m, 2H), 2.29-2.20 (m, |
| | | | 3H), 2.12-2.06 (m, 1H), 1.95-1.88 (m, 2H), 1.84-1.76 (m, 4H), |
| | | | 1.67-1.52 (m, 6H), 1.40-1.27 (m, 4H), 1.23-1.05 (m, 4H) |
| 298 | 827 | 828 | δ 10.91 (s, 1H), 8.46 (d, J = 2.9 Hz, 1H), 8.14 (s, 1H), 8.10-8.01 |
| | | | (m, 0.3H), 7.96 (br s, 1H), 7.76-7.70 (m, 1H), 7.70-7.65 (m, |
| | | | 1H), 7.65-7.58 (m, 2H), 7.58-7.49 (m, 1H), 7.43 (br s, 1H), |
| | | | 7.35-7.29 (m, 1H), 7.03 (br s, 1H), 6.52 (d, J = 9.8 Hz, 1H), 6.36 |
| | | | (t, J = 6.8 Hz, 1H), 4.43-4.24 (m, 2H), 3.97 (s, 3H), 3.95 (br d, |
| | | | J = 2.9 Hz, 1H), 3.85-3.72 (m, 1H), 3.25-3.06 (m, 2H), 2.93 (br |
| | | | s, 1H), 2.80-2.55 (m, 5H), 2.40-2.23 (m, 3H), 2.20-2.10 (m, |
| | | | 1H), 2.03-1.72 (m, 8H), 1.71-1.54 (m, 3H), 1.52-1.16 (m, 7H), |
| | | | 1.10-0.80 (m, 2H) |
| 299 | 831 | 832 | δ (DMSO-d6) 11.19 (s, 1H), 8.46 (d, J = 3.5 Hz, 1H), 8.05 (br d, |
| | | | J = 7.5 Hz, 1H), 7.99 (s, 1H), 7.80-7.75 (m, 1H), 7.74-7.68 (m, |
| | | | 2H), 7.61 (br d, J = 8.0 Hz, 1H), 7.56 (ddd, J = 9.1, 6.7, 2.0 Hz, |
| | | | 1H), 7.30 (br d, J = 8.6 Hz, 1H), 7.10-7.05 (m, 2H), 6.81-6.75 |
| | | | (m, 1H), 6.52 (d, J = 9.1 Hz, 1H), 6.36 (t, J = 6.4 Hz, 1H), 5.34- |
| | | | 5.26 (m, 1H), 3.76-3.64 (m, 3H), 3.06-2.99 (m, 3H), 2.95-2.88 |
| | | | (m, 3H), 2.68-2.62 (m, 4H), 2.27 (br d, J = 11.3 Hz, 2H), 2.16- |
| | | | 2.11 (m, 1H), 1.95-1.77 (m, 6H), 1.67-1.56 (m, 5H), 1.42-1.28 |
| | | | (m, 4H), 1.24-1.14 (m, 3H) |
| 300 | 861 | 862 | δ (DMSO-d6) 11.10-11.06 (m, 1H), 8.50-8.42 (m, 1H), 8.20 |
| | | | (s, 1H), 7.98 (br s, 1H), 7.81-7.73 (m, 3H), 7.61 (br d, J = 7.4 |
| | | | Hz, 2H), 7.33 (br t, J = 8.7 Hz, 2H), 7.09 (s, 1H), 7.01-6.98 (m, |
| | | | 1H), 6.90 (br d, J = 7.5 Hz, 1H), 5.34 (dd, J = 12.9, 5.3 Hz, 1H), |
| | | | 4.35-4.22 (m, 1H), 4.02-3.95 (m, 1H), 3.92-3.82 (m, 1H), 3.40- |
| | | | 3.33 (m, 3H), 3.20-3.09 (m, 2H), 3.05-2.92 (m, 5H), 2.81- |
| | | | 2.61 (m, 3H), 2.58-2.52 (m, 3H), 2.36-2.23 (m, 4H), 2.02-1.87 |
| | | | (m, 5H), 1.79-1.63 (m, 7H), 1.46 (br d, J = 8.3 Hz, 3H), 1.38- |
| | | | 1.28 (m, 1H) |
| 301 | 858 | 859 | δ (DMSO-d6) 11.12 (s, 1H), 8.48 (d, J = 3.6 Hz, 1H), 8.20 (s, |
| | | | 1H), 8.07 (br d, J = 7.6 Hz, 1H), 7.99 (br s, 1H), 7.79-7.67 (m, |
| | | | 2H), 7.66-7.52 (m, 2H), 7.49 (br d, J = 8.3 Hz, 1H), 7.27 (br d, |
| | | | J = 7.8 Hz, 1H), 7.11 (s, 1H), 7.02 (d, J = 8.1 Hz, 1H), 6.93 (d, |
| | | | J = 8.3 Hz, 1H), 6.53 (d, J = 9.1 Hz, 1H), 6.37 (dt, J = 6.7, 1.1 Hz, |
| | | | 1H), 5.36 (dd, J = 12.8, 5.3 Hz, 1H), 3.56-3.52 (m, 1H), 3.76- |
| | | | 3.51 (m, 3H), 3.07-2.94 (m, 3H), 2.93-2.86 (m, 3H), 2.85-2.55 |
| | | | (m, 5H), 2.22 (br d, J = 7.0 Hz, 2H), 2.12-1.94 (m, 7H), 1.88- |
| | | | 1.64 (m, 8H), 1.61-1.47 (m, 1H), 1.45-1.28 (m, 4H), 1.27-1.13 |
| | | | (m, 2H) |
| 302 | 845 | 846 | δ (DMSO-d6) 11.22 (s, 1H), 8.47 (d, J = 3.5 Hz, 1H), 8.17 (s, |
| | | | 1H), 8.05 (br d, J = 7.8 Hz, 1H), 7.97 (br s, 1H), 7.76-7.67 (m, |
| | | | 2H), 7.63-7.52 (m, 2H), 7.45 (br d, J = 8.4 Hz, 1H), 7.31 (s, 1H), |
| | | | 7.25 (br d, J = 7.8 Hz, 1H), 7.20-7.15 (m, 1H), 7.13-7.08 (m, |
| | | | 1H), 6.52 (d, J = 9.1 Hz, 1H), 6.39-6.33 (m, 1H), 5.39-5.32 (m, |
| | | | 1H), 3.71-3.64 (m, 1H), 3.57 (br d, J = 4.4 Hz, 1H), 2.94 (br d, |
| | | | J = 10.8 Hz, 2H), 2.90-2.84 (m, 3H), 2.78 (br d, J = 10.8 Hz, |
| | | | 2H), 2.72-2.61 (m, 2H), 2.20-2.11 (m, 3H), 2.05-1.92 (m, 6H), |
| | | | 1.82-1.63 (m, 9H), 1.57-1.44 (m, 1H), 1.40-1.31 (m, 4H), 1.23- |
| | | | 1.12 (m, 2H) |
| 303 | 831 | 832 | δ (DMSO-d6) 11.22 (s, 1H), 8.49 (d, J = 3.6 Hz, 1H), 8.19-8.16 |
| | | | (m, 1H), 8.06 (br d, J = 7.6 Hz, 1H), 7.99 (s, 1H), 7.75-7.67 (m, |
| | | | 2H), 7.63-7.59 (m, 1H), 7.57-7.50 (m, 1H), 7.42 (br d, J = 7.6 |
| | | | Hz, 1H), 7.31 (s, 1H), 7.19-7.14 (m, 1H), 7.10 (d, J = 7.8 Hz, |
| | | | 1H), 6.54-6.49 (m, 1H), 6.39-6.33 (m, 1H), 5.39-5.31 (m, 1H), |
| | | | 4.29-4.21 (m, 1H), 4.10-4.02 (m, 1H), 4.01-3.91 (m, 1H), 3.15- |
| | | | 3.08 (m, 1H), 3.02-2.88 (m, 4H), 2.86-2.76 (m, 3H), 2.71- |
| | | | 2.61 (m, 2H), 2.18-2.11 (m, 3H), 2.03-1.86 (m, 7H), 1.77-1.61 |
| | | | (m, 7H), 1.54-1.44 (m, 2H), 1.36-1.26 (m, 1H), 1.19-1.05 (m, |
| | | | 2H) |
| 304 | 843 | 844 | δ (DMSO-d6) 10.89 (br s, 1H), 8.47 (br d, J = 3.3 Hz, 1H), 8.22 |
| | | | (br s, 1H), 8.09-8.03 (m, 1H), 7.99 (br s, 1H), 7.75-7.66 (m, |
| | | | 2H), 7.62 (br d, J = 7.6 Hz, 2H), 7.57-7.52 (m, 1H), 7.45 (br s, |
| | | | 2H), 7.24 (br d, J = 7.6 Hz, 1H), 7.05 (br d, J = 8.1 Hz, 1H), 6.57- |
| | | | 6.50 (m, 1H), 6.40-6.32 (m, 1H), 4.34 (br dd, J = 9.2, 4.6 Hz, |
| | | | 1H), 3.98 (br d, J = 3.6 Hz, 3H), 3.00-2.97 (m, 2H), 2.88 (br s, |
| | | | 2H), 2.80 (br dd, J = 9.5, 1.1 Hz, 2H), 2.68-2.61 (m, 4H), 2.37- |
| | | | 2.32 (m, 2H), 2.21 (br s, 4H), 2.08 (br d, J = 4.3 Hz, 2H), 2.05 (br |
| | | | d, J = 1.3 Hz, 3H), 1.98 (br d, J = 1.1 Hz, 2H), 1.80 (br d, J = 0.8 |
| | | | Hz, 3H), 1.70 (br s, 2H), 1.55-1.49 (m, 2H), 1.37 (br s, 3H), 1.23- |
| | | | 1.16 (m, 3H) |
| 305 | 793 | 794 | δ (DMSO-d6) 10.80 (s, 1H), 9.04 (br d, J = 7.3 Hz, 1H), 8.47 (d, |
| | | | J = 3.5 Hz, 1H), 8.05 (br d, J = 7.6 Hz, 1H), 7.98 (br s, 1H), 7.88 |
| | | | (br d, J = 7.6 Hz, 1H), 7.75-7.68 (m, 2H), 7.61 (br d, J = 8.0 Hz, |
| | | | 1H), 7.58-7.52 (m, 1H), 7.28-7.20 (m, 1H), 6.87 (br t, J = 9.3 |
| | | | Hz, 1H), 6.54 (s, 1H), 6.52-6.50 (m, 1H), 6.43 (br d, J = 8.5 Hz, |
| | | | 1H), 6.36 (t, J = 6.8 Hz, 1H), 4.27 (br dd, J = 11.4, 4.6 Hz, 1H), |
| | | | 3.26 (br d, J = 10.8 Hz, 3H), 3.02-2.92 (m, 2H), 2.79-2.64 (m, |
| | | | 3H), 2.62 (br d, J = 13.1 Hz, 2H), 2.57-2.52 (m, 2H), 2.40-2.32 |
| | | | (m, 1H), 2.09 (br d, J = 9.1 Hz, 3H), 1.96 (br s, 3H), 1.91 (br s, |
| | | | 2H), 1.87 (br d, J = 3.8 Hz, 1H), 1.86-1.76 (m, 6H), 1.36-1.25 |
| | | | (m, 4H) |
| 306 | 830 | 831 | δ (DMSO-d6) 11.25 (s, 1H), 8.48 (d, J = 3.6 Hz, 1H), 8.06 (br d, |
| | | | J = 7.3 Hz, 1H), 8.00 (s, 1H), 7.72 (br d, J = 7.9 Hz, 2H), 7.64- |
| | | | 7.60 (m, 1H), 7.57 (ddd, J = 9.1, 6.8, 2.0 Hz, 1H), 7.40-7.29 (m, |
| | | | 3H), 7.25 (br d, J = 8.5 Hz, 1H), 7.11 (br d, J = 9.4 Hz, 1H), 6.54 |
| | | | (d, J = 9.4 Hz, 1H), 6.38 (t, J = 6.7 Hz, 1H), 5.38 (dd, J = 13.1, |
| | | | 5.3 Hz, 1H), 5.07 (s, 1H), 3.78-3.70 (m, 1H), 3.67-3.57 (m, |
| | | | 2H), 3.47 (s, 1H), 3.12-2.94 (m, 4H), 2.93-2.82 (m, 2H), 2.77- |
| | | | 2.61 (m, 2H), 2.28-2.13 (m, 2H), 1.98 (br s, 1H), 1.93 (br d, |
| | | | J = 10.6 Hz, 3H), 1.85-1.73 (m, 6H), 1.73-1.58 (m, 2H), 1.42- |
| | | | 1.24 (m, 4H), 1.22-1.14 (m, 2H), 1.11-0.99 (m, 2H) |
| 307 | 827 | 828 | δ 10.90 (s, 1H), 8.46 (d, J = 3.5 Hz, 1H), 8.10-8.02 (m, 1H), 8.00 |
| | | | (s, 1H), 7.74-7.66 (m, 4H), 7.62-7.52 (m, 2H), 7.42 (br s, 1H), |
| | | | 7.30 (br d, J = 7.7 Hz, 1H), 7.05 (br d, J = 8.3 Hz, 1H), 6.53 (br |
| | | | d, J = 9.3 Hz, 1H), 6.37 (br t, J = 6.6 Hz, 1H), 4.35 (br dd, J = 9.8, |
| | | | 5.0 Hz, 1H), 4.05-3.92 (m, 3H), 3.81-3.67 (m, 1H), 3.48 (br s, |
| | | | 3H), 3.01-2.79 (m, 4H), 2.74-2.57 (m, 2H), 2.53-2.51 (m, 1H), |
| | | | 2.42-2.30 (m, 1H), 2.26-2.11 (m, 2H), 2.00 (br s, 4H), 1.95- |
| | | | 1.87 (m, 2H), 1.80 (br s, 5H), 1.71 (br s, 1H), 1.64 (br d, J = 8.9 |
| | | | Hz, 1H), 1.43-1.26 (m, 3H), 1.20 (br d, J = 9.8 Hz, 3H), 1.11- |
| | | | 0.96 (m, 2H) |
| 308 | 806 | 807 | δ (DMSO-d6)10.82 (s, 1H), 8.47 (d, J = 3.4 Hz, 1H), 8.10-8.03 |
| | | | (m, 1H), 8.00-7.93 (m, 1H), 7.76-7.72 (m, 1H), 7.71-7.67 (m, |
| | | | 1H), 7.61 (br d, J = 7.9 Hz, 1H), 7.58-7.52 (m, 1H), 7.32 (br d, |
| | | | J = 8.2 Hz, 1H), 7.05-6.95 (m, 1H), 6.53 (d, J = 9.3 Hz, 1H), |
| | | | 6.47-6.41 (m, 2H), 6.39-6.33 (m, 1H), 6.02 (br d, J = 7.6 Hz, |
| | | | 1H), 4.41-4.27 (m, 2H), 3.98-3.89 (m, 1H), 3.84-3.76 (m, 1H), |
| | | | 3.00-2.89 (m, 3H), 2.79-2.70 (m, 2H), 2.59 (br d, J = 4.0 Hz, |
| | | | 2H), 2.36-2.24 (m, 2H), 2.12-2.05 (m, 1H), 2.00-1.81 (m, 6H), |
| | | | 1.78-1.73 (m, 1H), 1.67-1.58 (m, 6H), 1.49-1.16 (m, 8H), 1.07- |
| | | | 0.83 (m, 2H) |
| 309 | 806 | 807 | δ (DMSO-d6) 10.83-10.76 (m, 1H), 9.11-8.97 (m, 1H), 8.50- |
| | | | 8.43 (m, 1H), 8.12-8.01 (m, 1H), 8.01-7.93 (m, 1H), 7.76-7.67 |
| | | | (m, 2H), 7.65-7.53 (m, 2H), 7.37-7.26 (m, 1H), 6.98-6.89 (m, |
| | | | 1H), 6.57-6.44 (m, 3H), 6.41-6.33 (m, 1H), 4.41-4.31 (m, 2H), |
| | | | 3.97-3.92 (m, 2H), 3.57 (br d, J = 10.4 Hz, 2H), 3.09-3.02 (m, |
| | | | 2H), 2.96-2.84 (m, 2H), 2.79-2.67 (m, 2H), 2.15-2.03 (m, 1H), |
| | | | 1.99-1.82 (m, 8H), 1.70-1.49 (m, 7H), 1.46-1.37 (m, 2H), 1.37- |
| | | | 1.21 (m, 4H), 1.09-0.87 (m, 3H) |
| 310 | 842 | 843 | δ (DMSO-d6) 10.98-10.78 (m, 1H), 8.56-8.47 (m, 1H), 8.12 |
| | | | (br d, J = 6.3 Hz, 1H), 8.06-7.98 (m, 1H), 7.84-7.71 (m, 2H), |
| | | | 7.70-7.56 (m, 2H), 7.36 (br d, J = 6.6 Hz, 1H), 7.17-7.07 (m, |
| | | | 1H), 6.61-6.48 (m, 3H), 6.42 (br t, J = 6.7 Hz, 1H), 6.24 (br d, |
| | | | J = 7.4 Hz, 1H), 4.48-4.34 (m, 2H), 4.06-3.92 (m, 1H), 3.91- |
| | | | 3.78 (m, 1H), 3.29-3.13 (m, 2H), 3.10-2.89 (m, 3H), 2.86-2.71 |
| | | | (m, 3H), 2.23-2.08 (m, 3H), 2.04-1.91 (m, 3H), 1.87-1.78 (m, |
| | | | 2H), 1.77-1.62 (m, 4H), 1.60-1.23 (m, 8H), 1.17-0.84 (m, 3H) |
| 311 | 842 | 843 | δ (DMSO-d6) 10.84 (s, 1H), 8.46 (br s, 1H), 8.06 (br d, J = 7.5 |
| | | | Hz, 1H), 7.97 (br s, 1H), 7.77-7.66 (m, 2H), 7.65-7.53 (m, 2H), |
| | | | 7.32 (br d, J = 7.1 Hz, 1H), 7.03 (br d, J = 7.0 Hz, 1H), 6.57-6.47 |
| | | | (m, 3H), 6.41-6.24 (m, 2H), 4.41-4.30 (m, 2H), 4.07-3.89 (m, |
| | | | 2H), 3.86-3.74 (m, 1H), 3.70-3.50 (m, 3H), 3.02-2.85 (m, 1H), |
| | | | 2.83-2.65 (m, 2H), 2.32-2.16 (m, 1H), 2.13-2.01 (m, 2H), 1.99- |
| | | | 1.82 (m, 4H), 1.80-1.73 (m, 1H), 1.71-1.45 (m, 7H), 1.44- |
| | | | 1.14 (m, 6H), 1.11-0.82 (m, 3H) |
| 312 | 844 | 845 | δ (DMSO-d6) 11.15-11.07 (m, 1H), 8.50 (d, J = 3.6 Hz, 1H), |
| | | | 8.29-8.21 (m, 1H), 8.13-8.04 (m, 1H), 8.01-7.98 (m, 1H), 7.77- |
| | | | 7.68 (m, 2H), 7.65-7.59 (m, 1H), 7.55 (ddd, J = 2.1, 6.8, 9.1 |
| | | | Hz, 1H), 7.47-7.40 (m, 1H), 7.14-7.08 (m, 1H), 7.04-6.98 (m, |
| | | | 1H), 6.97-6.89 (m, 1H), 6.53 (d, J = 9.3 Hz, 1H), 6.40-6.33 (m, |
| | | | 1H), 5.38-5.32 (m, 1H), 4.31-4.22 (m, 1H), 4.13-4.03 (m, 1H), |
| | | | 4.02-3.92 (m, 1H), 3.22-3.04 (m, 2H), 3.02-2.86 (m, 4H), 2.85- |
| | | | 2.66 (m, 4H), 2.21-2.09 (m, 3H), 2.05-1.84 (m, 8H), 1.82- |
| | | | 1.64 (m, 7H), 1.60-1.40 (m, 3H), 1.39-1.03 (m, 4H) |
| 313 | 821 | 822 | δ (DMSO-d6) 10.82 (s, 1H), 8.48 (d, J = 3.6 Hz, 1H), 8.19 (s, |
| | | | 1H), 8.07 (br d, J = 7.3 Hz, 1H), 7.99 (s, 1H), 7.78-7.67 (m, 2H), |
| | | | 7.65-7.52 (m, 2H), 7.45 (d, J = 8.3 Hz, 1H), 7.27 (br d, J = 7.6 |
| | | | Hz, 1H), 7.04-6.96 (m, 1H), 6.53 (d, J = 9.1 Hz, 1H), 6.49-6.41 |
| | | | (m, 2H), 6.37 (dt, J = 1.2, 6.7 Hz, 1H), 6.03 (d, J = 8.0 Hz, 1H), |
| | | | 4.37-4.28 (m, 1H), 3.74-3.63 (m, 1H), 3.62-3.53 (m, 1H), 2.93 |
| | | | (br d, J = 10.9 Hz, 2H), 2.87 (s, 2H), 2.81-2.69 (m, 3H), 2.63- |
| | | | 2.55 (m, 2H), 2.17 (br d, J = 7.0 Hz, 2H), 2.12-1.94 (m, 7H), |
| | | | 1.92-1.76 (m, 3H), 1.72-1.60 (m, 6H), 1.54-1.46 (m, 1H), 1.41- |
| | | | 1.30 (m, 4H), 1.24-1.12 (m, 2H) |
| 314 | 807 | 808 | δ (DMSO-d6) 10.81 (br s, 1H), 8.48 (d, J = 3.6 Hz, 1H), 8.27 (s, |
| | | | 1H), 8.06 (br d, J = 7.6 Hz, 1H), 7.99 (s, 1H), 7.74-7.67 (m, 2H), |
| | | | 7.64-7.58 (m, 1H), 7.54 (ddd, J = 9.0, 6.7, 2.0 Hz, 1H), 7.41 (br |
| | | | d, J = 7.6 Hz, 1H), 6.98 (s, 1H), 6.52 (d, J = 9.1 Hz, 1H), 6.46 (br |
| | | | s, 1H), 6.42 (s, 1H), 6.38-6.34 (m, 1H), 6.02 (br d, J = 7.1 Hz, |
| | | | 1H), 4.30 (br dd, J = 11.2, 5.6 Hz, 2H), 4.07-3.93 (m, 2H), 3.32- |
| | | | 3.22 (m, 2H), 3.16-3.03 (m, 2H), 2.95 (br s, 2H), 2.84 (br d, |
| | | | J = 11.3 Hz, 2H), 2.78-2.70 (m, 2H), 2.59 (br d, J = 4.1 Hz, 2H), |
| | | | 2.57-2.53 (m, 1H), 2.21 (br d, J = 6.8 Hz, 1H), 2.18 (br d, J = |
| | | | 6.6 Hz, 1H), 2.06-2.00 (m, 4H), 1.90 (br dd, J = 12.3, 4.3 Hz, |
| | | | 2H), 1.86-1.82 (m, 2H), 1.65 (br d, J = 3.5 Hz, 4H), 1.53-1.47 |
| | | | (m, 1H), 1.31-1.10 (m, 4H) |
| 315 | 828 | 829 | δ (DMSO-d6) 10.90 (br s, 1H), 8.49 (d, J = 3.5 Hz, 1H), 8.06 (br |
| | | | d, J = 7.4 Hz, 1H), 7.99 (s, 1H), 7.75-7.67 (m, 2H), 7.60 (br d, |
| | | | J = 8.4 Hz, 2H), 7.57-7.51 (m, 1H), 7.46-7.40 (m, 2H), 7.03 (d, |
| | | | J = 8.6 Hz, 1H), 6.52 (d, J = 9.3 Hz, 1H), 6.36 (t, J = 6.5 Hz, 1H), |
| | | | 4.33 (dd, J = 9.4, 4.9 Hz, 1H), 4.25 (br d, J = 11.9 Hz, 1H), 4.07 |
| | | | (br d, J = 12.1 Hz, 1H), 3.97 (s, 4H), 3.24 (br d, J = 13.5 Hz, 1H), |
| | | | 3.16-3.07 (m, 1H), 3.00-2.92 (m, 3H), 2.84-2.75 (m, 3H), 2.68- |
| | | | 2.59 (m, 3H), 2.40-2.31 (m, 1H), 2.16 (br d, J = 7.5 Hz, 3H), |
| | | | 2.00-1.93 (m, 4H), 1.92 (br d, J = 3.3 Hz, 2H), 1.78 (br s, 4H), |
| | | | 1.74-1.68 (m, 2H), 1.55-1.45 (m, 2H), 1.34-1.27 (m, 1H), 1.18- |
| | | | 1.07 (m, 2H) |
| 316 | 821 | 822 | δ (DMSO-d6) 10.82 (s, 1H), 9.06-8.94 (m, 1H), 8.47 (d, J = 3.5 |
| | | | Hz, 1H), 8.05 (br d, J = 7.3 Hz, 1H), 7.97 (br s, 1H), 7.89 (br d, |
| | | | J = 8.1 Hz, 1H), 7.75-7.71 (m, 1H), 7.71-7.67 (m, 1H), 7.63- |
| | | | 7.59 (m, 1H), 7.55 (ddd, J = 2.0, 6.8, 9.1 Hz, 1H), 7.28-7.23 (m, |
| | | | 1H), 6.62-6.42 (m, 4H), 6.39-6.33 (m, 1H), 4.32 (br s, 1H), |
| | | | 3.71-3.60 (m, 2H), 3.54 (br d, J = 12.8 Hz, 3H), 3.34-3.22 (m, |
| | | | 2H), 3.16-3.07 (m, 3H), 2.91-2.85 (m, 2H), 2.79-2.69 (m, 2H), |
| | | | 2.10-2.05 (m, 1H), 1.98 (br dd, J = 3.6, 7.4 Hz, 2H), 1.88 (br d, |
| | | | J = 11.9 Hz, 2H), 1.80 (br d, J = 9.9 Hz, 6H), 1.70-1.58 (m, 3H), |
| | | | 1.51-1.40 (m, 3H), 1.35-1.24 (m, 5H) |
| 317 | 817 | 818 | δ (DMSO-d6) 11.23 (s, 1H), 8.49 (br d, J = 3.3 Hz, 1H), 8.07 (br |
| | | | d, J = 7.3 Hz, 1H), 7.99 (br s, 1H), 7.91 (br d, J = 7.1 Hz, 1H), |
| | | | 7.76-7.69 (m, 2H), 7.62 (br d, J = 7.6 Hz, 1H), 7.59-7.54 (m, |
| | | | 1H), 7.34-7.24 (m, 1H), 7.17-7.12 (m, 1H), 6.88-6.81 (m, 1H), |
| | | | 6.58-6.50 (m, 1H), 6.38 (br t, J = 6.7 Hz, 1H), 5.36-5.28 (m, |
| | | | 1H), 3.82 (br d, J = 10.5 Hz, 2H), 3.72-3.63 (m, 1H), 3.62-3.49 |
| | | | (m, 3H), 3.39-3.33 (m, 1H), 3.02-2.88 (m, 3H), 2.77-2.66 (m, |
| | | | 4H), 2.65 (br s, 2H), 2.42-2.32 (m, 1H), 2.19-2.07 (m, 3H), |
| | | | 2.01-1.94 (m, 2H), 1.92 (br s, 1H), 1.88-1.68 (m, 6H), 1.41- |
| | | | 1.24 (m, 4H) |
| 318 | 792 | 793 | δ (DMSO-d6) 10.81 (s, 1H), 8.47 (br d, J = 2.1 Hz, 1H), 8.14 (s, |
| | | | 1H), 8.07 (br d, J = 6.6 Hz, 1H), 7.98 (br s, 1H), 7.76-7.67 (m, |
| | | | 2H), 7.63-7.52 (m, 2H), 7.31 (br d, J = 7.2 Hz, 1H), 6.98 (br t, |
| | | | J = 8.3 Hz, 1H), 6.55-6.42 (m, 3H), 6.36 (br t, J = 6.7 Hz, 1H), |
| | | | 6.07 (br d, J = 7.7 Hz, 1H), 4.44-4.36 (m, 1H), 4.35-4.29 (m, |
| | | | 1H), 3.96 (br d, J = 11.9 Hz, 1H), 3.71-3.60 (m, 1H), 3.34 (br s, |
| | | | 4H), 3.07-2.96 (m, 1H), 2.79-2.70 (m, 2H), 2.60 (br d, J = 2.6 |
| | | | Hz, 2H), 2.56 (br s, 2H), 2.12-2.04 (m, 1H), 2.02-1.96 (m, 2H), |
| | | | 1.94-1.85 (m, 2H), 1.80 (br d, J = 3.3 Hz, 1H), 1.79-1.70 (m, |
| | | | 6H), 1.69 (br s, 1H), 1.52-1.42 (m, 2H), 1.40-1.21 (m, 3H), |
| | | | 1.12-0.91 (m, 2H) |
| 319 | 793 | 794 | δ (DMSO-d6) 10.80 (s, 1H), 9.41-9.29 (m, 1H), 8.47 (d, J = 3.6 |
| | | | Hz, 1H), 8.05 (br d, J = 7.5 Hz, 1H), 7.97 (s, 1H), 7.74-7.68 (m, |
| | | | 3H), 7.60 (br d, J = 7.9 Hz, 1H), 7.55 (ddd, J = 9.1, 6.7, 2.1 Hz, |
| | | | 1H), 7.24 (br d, J = 7.5 Hz, 1H), 6.89 (t, J = 9.3 Hz, 1H), 6.58- |
| | | | 6.50 (m, 2H), 6.48-6.43 (m, 1H), 6.36 (dt, J = 1.1, 6.7 Hz, 1H), |
| | | | 4.29 (dd, J = 4.9, 11.4 Hz, 1H), 3.72-3.57 (m, 2H), 3.56-3.50 |
| | | | (m, 2H), 3.30 (br d, J = 11.9 Hz, 2H), 3.22-3.13 (m, 2H), 3.02- |
| | | | 2.94 (m, 2H), 2.78-2.67 (m, 1H), 2.15 (br s, 2H), 2.11-2.04 (m, |
| | | | 2H), 1.96 (br d, J = 7.4 Hz, 2H), 1.91-1.83 (m, 3H), 1.81-1.77 |
| | | | (m, 2H), 1.44 (br t, J = 8.8 Hz, 4H), 1.29-1.23 (m, 6H) |
| 320 | 831 | 832 | δ (DMSO-d6) 11.23-11.21 (m, 1H), 8.48 (d, J = 3.4 Hz, 1H), |
| | | | 8.05 (s, 1H), 8.02 (s, 1H), 7.96 (br d, J = 6.5 Hz, 1H), 7.77-7.73 |
| | | | (m, 1H), 7.73-7.69 (m, 1H), 7.65-7.61 (m, 1H), 7.60-7.55 (m, |
| | | | 1H), 7.35 (br d, J = 8.2 Hz, 1H), 7.14-7.08 (m, 2H), 6.83 (s, 1H), |
| | | | 6.56 (s, 1H), 6.41-6.35 (m, 1H), 5.36-5.28 (m, 1H), 3.69-3.65 |
| | | | (m, 3H), 3.56-3.51 (m, 2H), 3.04-2.98 (m, 3H), 2.92-2.85 (m, |
| | | | 1H), 2.70-2.68 (m, 2H), 2.31-2.10 (m, 3H), 2.00-1.89 (m, 6H), |
| | | | 1.81 (br dd, J = 3.2, 11.4 Hz, 3H), 1.71-1.62 (m, 3H), 1.43-1.19 |
| | | | (m, 8H) |
| 321 | 830 | 831 | δ (DMSO-d6) 11.24-11.22 (m, 1H), 9.27-9.11 (m, 1H), 8.47 |
| | | | (d, J = 3.6 Hz, 1H), 8.10-8.03 (m, 1H), 7.98 (s, 1H), 7.76-7.67 |
| | | | (m, 2H), 7.63-7.52 (m, 2H), 7.31 (s, 1H), 7.23 (d, J = 8.1 Hz, |
| | | | 1H), 7.10 (br d, J = 9.5 Hz, 1H), 6.55-6.49 (m, 1H), 6.39-6.32 |
| | | | (m, 1H), 5.40-5.34 (m, 1H), 4.45-4.37 (m, 1H), 4.00-3.93 (m, |
| | | | 1H), 3.71-3.56 (m, 3H), 3.40-3.27 (m, 1H), 3.19-3.10 (m, 2H), |
| | | | 3.09-2.96 (m, 3H), 2.93-2.84 (m, 2H), 2.75-2.62 (m, 2H), 2.56 |
| | | | (br d, J = 12.4 Hz, 1H), 2.20-2.12 (m, 1H), 2.07-1.95 (m, 4H), |
| | | | 1.90-1.79 (m, 2H), 1.76-1.56 (m, 7H), 1.51-1.42 (m, 2H), 1.40- |
| | | | 1.31 (m, 2H), 1.16-1.07 (m, 1H), 0.99 (br d, J = 9.6 Hz, 1H) |
| 322 | 806 | 807 | δ (DMSO-d6) 10.81 (s, 1H), 8.47 (d, J = 3.5 Hz, 1H), 8.07 (br d, |
| | | | J = 7.5 Hz, 1H), 7.98 (s, 1H), 7.76-7.66 (m, 2H), 7.64-7.50 (m, |
| | | | 2H), 7.30 (br d, J = 7.5 Hz, 1H), 6.97 (br t, J = 8.8 Hz, 1H), 6.54- |
| | | | 6.41 (m, 3H), 6.36 (dt, J = 6.7, 1.0 Hz, 1H), 6.08 (br d, J = 7.6 |
| | | | Hz, 1H), 4.45-4.27 (m, 2H), 4.00-3.89 (m, 1H), 3.75-3.56 (m, |
| | | | 1H), 2.99 (br t, J = 12.1 Hz, 1H), 2.91-2.65 (m, 5H), 2.57 (br dd, |
| | | | J = 17.4, 4.2 Hz, 3H), 2.08 (td, J = 8.5, 4.3 Hz, 1H), 1.99 (br d, |
| | | | J = 8.0 Hz, 2H), 1.84-1.63 (m, 10H), 1.57-1.41 (m, 6H), 1.40- |
| | | | 1.27 (m, 3H), 1.13-1.02 (m, 1H), 0.96 (br d, J = 9.5 Hz, 1H) |
| 323 | 816 | 817 | δ (DMSO-d6) 11.22 (s, 1H), 8.49-8.45 (m, 1H), 8.10-8.04 (m, |
| | | | 1H), 8.00-7.97 (m, 1H), 7.75-7.67 (m, 2H), 7.63-7.51 (m, 2H), |
| | | | 7.33-7.21 (m, 3H), 7.14-7.08 (m, 1H), 6.52 (d, J = 9.1 Hz, 1H), |
| | | | 6.39-6.33 (m, 1H), 5.41-5.33 (m, 1H), 4.47-4.39 (m, 1H), 4.05- |
| | | | 3.96 (m, 2H), 3.11-2.99 (m, 5H), 2.94-2.84 (m, 2H), 2.74- |
| | | | 2.64 (m, 2H), 2.63-2.53 (m, 2H), 2.20-2.10 (m, 2H), 2.06-1.89 |
| | | | (m, 7H), 1.82 (br d, J = 8.1 Hz, 3H), 1.71 (br d, J = 11.9 Hz, 2H), |
| | | | 1.54-1.42 (m, 2H), 1.40-1.29 (m, 2H), 1.19-1.12 (m, 1H), 1.08- |
| | | | 1.01 (m, 1H) |
| 324 | 806 | 807 | δ 8.21-8.15 (m, 2H), 8.07-8.05 (br d, J = 7.9 Hz, 1H), 7.82-7.81 |
| | | | (m, 1H), 7.56-7.51 (m, 1H), 7.44-7.42 (br d, J = 8.0 Hz, 1H), |
| | | | 7.38-7.32 (m, 2H), 7.04-7.00 (m, 1H), 6.61-6.60 (br d, J = 8.4 |
| | | | Hz, 1H), 6.36-6.34 (m, 1H), 6.28-6.19 (m, 2H), 4.98-4.94 (m, |
| | | | 1H), 4.64-4.62 (m, 1H), 3.98-3.93 (m, 1H), 3.82-3.56 (m, 2H), |
| | | | 2.89-2.79 (m, 2H), 2.74-2.59 (m, 2H), 2.50-2.43 (m, 1H), 2.27- |
| | | | 2.22 (m, 1H), 2.18-2.04 (m, 2H), 2.01-1.99 (m, 1H), 1.94- |
| | | | 1.75 (m, 10H), 1.71-1.65 (m, 3H), 1.43-1.30 (m, 3H), 1.28- |
| | | | 1.25 (br d, J = 11.1 Hz, 1H), 1.22-1.14 (m, 1H), 1.10-0.89 (m, |
| | | | 4H) |
| 325 | 802 | 803 | δ 11.23 (s, 1H), 8.46 (d, J = 3.6 Hz, 1H), 8.14 (s, 0.2H), 8.06 (br |
| | | | d, J = 7.4 Hz, 1H), 7.97 (s, 1H), 7.75-7.69 (m, 2H), 7.69-7.64 |
| | | | (m, 1H), 7.60 (dd, J = 7.9, 0.9 Hz, 1H), 7.55 (ddd, J = 9.1, 6.8, |
| | | | 2.0 Hz, 1H), 7.31 (br s, 1H), 7.20 (d, J = 8.3 Hz, 1H), 7.09 (br d, |
| | | | J = 8.9 Hz, 1H), 6.52 (d, J = 9.1 Hz, 1H), 6.36 (dt, J = 6.7, 1.0 Hz, |
| | | | 1H), 5.36 (br dd, J = 12.9, 5.2 Hz, 1H), 4.39-4.19 (m, 2H), 3.77 |
| | | | (br d, J = 13.3 Hz, 1H), 3.43-3.37 (m, 2H), 3.06-2.98 (m, 1H), |
| | | | 2.97-2.84 (m, 3H), 2.82-2.58 (m, 5H), 2.55 (br s, 2H), 2.45 (br |
| | | | d, J = 7.3 Hz, 2H), 2.19-2.06 (m, 3H), 2.00-1.87 (m, 2H), 1.78 |
| | | | (br d, J = 11.9 Hz, 2H), 1.68 (br s, 2H), 1.53 (br s, 3H), 1.08- |
| | | | 0.88 (m, 2H) |
| 326 | 822 | 823 | δ 10.78 (br s, 1H), 8.46 (d, J = 3.6 Hz, 1H), 8.05 (br d, J = 7.5 Hz, |
| | | | 1H), 7.97 (s, 1H), 7.76-7.66 (m, 2H), 7.63-7.52 (m, 2H), 7.42 |
| | | | (br d, J = 8.4 Hz, 1H), 7.24 (br d, J = 7.9 Hz, 1H), 6.82 (t, J = 9.4 |
| | | | Hz, 1H), 6.56-6.47 (m, 2H), 6.44-6.31 (m, 2H), 5.80 (d, J = 7.8 |
| | | | Hz, 1H), 4.25 (ddd, J = 4.8, 7.2, 11.6 Hz, 1H), 3.73-3.51 (m, |
| | | | 2H), 2.85 (br s, 6H), 2.80-2.67 (m, 3H), 2.58 (br d, J = 4.1 Hz, |
| | | | 2H), 2.46 (br s, 3H), 2.17 (br d, J = 7.1 Hz, 2H), 2.12-1.92 (m, |
| | | | 5H), 1.91-1.73 (m, 3H), 1.67 (br d, J = 11.9 Hz, 2H), 1.48 (br d, |
| | | | J = 5.9 Hz, 1H), 1.41-1.27 (m, 4H), 1.24-1.11 (m, 2H) |
| 327 | 822 | 823 | δ 10.79 (s, 1H), 8.46 (d, J = 3.6 Hz, 1H), 8.15 (s, 0.3H), 8.04 (br |
| | | | d, J = 7.5 Hz, 1H), 7.96 (br s, 1H), 7.75-7.66 (m, 2H), 7.60 (br |
| | | | d, J = 7.9 Hz, 1H), 7.54 (ddd, J = 1.9, 6.8, 9.1 Hz, 1H), 7.47 (br |
| | | | d, J = 8.1 Hz, 1H), 7.25 (br d, J = 7.8 Hz, 1H), 6.82 (t, J = 9.3 Hz, |
| | | | 1H), 6.54-6.46 (m, 2H), 6.42-6.33 (m, 2H), 5.79 (br d, J = 7.6 |
| | | | Hz, 1H), 4.25 (ddd, J = 4.6, 7.3, 11.7 Hz, 1H), 3.72-3.48 (m, |
| | | | 3H), 3.10 (br d, J = 10.9 Hz, 3H), 2.87 (s, 2H), 2.81-2.64 (m, |
| | | | 2H), 2.42 (br d, J = 1.3 Hz, 7H), 2.17 (br d, J = 6.9 Hz, 3H), 2.08 |
| | | | (td, J = 4.2, 8.7 Hz, 1H), 1.96 (br s, 2H), 1.80-1.70 (m, 4H), 1.64- |
| | | | 1.50 (m, 1H), 1.44-1.13 (m, 7H) |
| 328 | 821 | 822 | δ 10.83-10.76 (m, 1H), 8.50-8.44 (m, 1H), 8.14 (s, 1H), 8.08- |
| | | | 8.02 (m, 1H), 7.98 (br s, 1H), 7.87 (br d, J = 2.9 Hz, 1H), 7.74- |
| | | | 7.68 (m, 2H), 7.63-7.59 (m, 1H), 7.56 (ddd, J = 9.1, 6.7, 2.1 Hz, |
| | | | 1H), 7.26-7.21 (m, 1H), 7.01-6.93 (m, 1H), 6.53 (d, J = 9.3 Hz, |
| | | | 1H), 6.51-6.44 (m, 2H), 6.37 (dt, J = 6.8, 1.3 Hz, 1H), 6.10 (br |
| | | | d, J = 6.6 Hz, 1H), 4.37-4.27 (m, 1H), 3.72-3.60 (m, 1H), 3.59- |
| | | | 3.46 (m, 1H), 3.17-3.05 (m, 3H), 2.93-2.82 (m, 2H), 2.80- |
| | | | 2.65 (m, 4H), 2.63-2.59 (m, 1H), 2.58-2.54 (m, 1H), 2.35-2.27 |
| | | | (m, 2H), 2.08 (dt, J = 8.8, 4.4 Hz, 2H), 1.98 (br d, J = 6.6 Hz, 4H), |
| | | | 1.92-1.77 (m, 7H), 1.69-1.57 (m, 2H), 1.41-1.19 (m, 6H) |
| 329 | 845 | 846 | δ (DMSO-d6) 8.45 (d, J = 3.7 Hz, 1H), 8.04 (br d, J = 7.2 Hz, |
| | | | 1H), 7.96 (br s, 1H), 7.74-7.66 (m, 2H), 7.62-7.51 (m, 3H), |
| | | | 7.22 (br d, J = 7.5 Hz, 1H), 7.03-7.00 (m, 1H), 7.00-6.95 (m, |
| | | | 1H), 6.75 (br d, J = 7.9 Hz, 1H), 6.54-6.49 (m, 1H), 6.35 (t, J = |
| | | | 6.5 Hz, 1H), 5.07-4.95 (m, 1H), 3.69-3.44 (m, 5H), 2.90-2.82 |
| | | | (m, 2H), 2.64-2.53 (m, 5H), 2.31-2.23 (m, 2H), 2.06-1.91 (m, |
| | | | 4H), 1.81-1.70 (m, 6H), 1.63-1.53 (m, 4H), 1.38 (br s, 3H), |
| | | | 1.31-1.21 (m, 6H) |
| 330 | 831 | 832 | δ (DMSO-d6) 11.23-11.12 (m, 1H), 8.49 (d, J = 3.5 Hz, 1H), |
| | | | 8.06 (br d, J = 7.5 Hz, 1H), 7.98 (s, 1H), 7.75-7.67 (m, 2H), 7.61 |
| | | | (br d, J = 8.1 Hz, 1H), 7.58-7.52 (m, 1H), 7.37 (br d, J = 7.4 Hz, |
| | | | 1H), 7.09-7.04 (m, 2H), 6.77 (dd, J = 2.0, 8.8 Hz, 1H), 6.52 (d, |
| | | | J = 9.3 Hz, 1H), 6.36 (t, J = 6.7 Hz, 1H), 5.29 (dd, J = 5.2, 13.2 |
| | | | Hz, 1H), 4.33-4.24 (m, 1H), 4.01-3.88 (m, 2H), 3.61-3.55 (m, |
| | | | 2H), 3.20-3.11 (m, 1H), 2.87 (br d, J = 12.0 Hz, 3H), 2.69-2.59 |
| | | | (m, 4H), 2.30 (br t, J = 5.9 Hz, 2H), 2.17-2.09 (m, 1H), 1.96- |
| | | | 1.85 (m, 4H), 1.75 (br d, J = 12.4 Hz, 2H), 1.57 (br d, J = 3.5 Hz, |
| | | | 4H), 1.40 (br s, 5H), 1.34-1.20 (m, 4H) |
| 331 | 861 | 862 | δ (DMSO-d6) 11.23 (s, 1H), 8.50 (d, J = 3.5 Hz, 1H), 8.18 (s, |
| | | | 1H), 8.07 (br d, J = 7.6 Hz, 1H), 8.01-7.98 (m, 1H), 7.72 (d, J = |
| | | | 8.2 Hz, 1H), 7.64-7.59 (m, 1H), 7.56 (ddd, J = 9.0, 6.8, 2.0 Hz, |
| | | | 1H), 7.43 (br d, J = 7.3 Hz, 1H), 7.32 (s, 1H), 7.21-7.14 (m, 1H), |
| | | | 7.14-7.08 (m, 1H), 6.53 (d, J = 9.2 Hz, 1H), 6.41-6.33 (m, 1H), |
| | | | 5.71 (br s, 1H), 5.41-5.33 (m, 1H), 4.89-4.62 (m, 1H), 4.40- |
| | | | 4.21 (m, 1H), 4.03-3.94 (m, 2H), 3.90-3.78 (m, 1H), 3.59-3.49 |
| | | | (m, 1H), 3.35 (br d, J = 12.5 Hz, 4H), 3.28-3.23 (m, 1H), 3.18- |
| | | | 3.06 (m, 2H), 2.95-2.89 (m, 2H), 2.74-2.62 (m, 2H), 2.26-2.08 |
| | | | (m, 3H), 2.01-1.89 (m, 3H), 1.81-1.72 (m, 4H), 1.72-1.64 (m, |
| | | | 3H), 1.51-1.24 (m, 3H) |
| 332 | 828 | 829 | δ (DMSO-d6) 10.89 (s, 1H), 8.49 (d, J = 3.5 Hz, 1H), 8.06 (br d, |
| | | | J = 7.6 Hz, 1H), 8.01 (s, 1H), 7.77-7.69 (m, 3H), 7.63 (br d, J = |
| | | | 7.9 Hz, 1H), 7.57 (br d, J = 7.0 Hz, 1H), 7.51 (d, J = 8.8 Hz, 1H), |
| | | | 7.35 (br d, J = 7.8 Hz, 1H), 6.94 (br d, J = 9.2 Hz, 1H), 6.86 (s, |
| | | | 1H), 6.54 (br d, J = 8.8 Hz, 1H), 6.39 (br t, J = 7.0 Hz, 1H), 4.28 |
| | | | (dd, J = 4.9, 9.4 Hz, 1H), 3.91 (s, 3H), 3.87-3.80 (m, 3H), 3.79- |
| | | | 3.71 (m, 2H), 2.81-2.73 (m, 2H), 2.67-2.61 (m, 2H), 2.21- |
| | | | 2.16 (m, 1H), 2.01-1.77 (m, 11H), 1.63 (br d, J = 2.1 Hz, 5H), |
| | | | 1.42-1.28 (m, 6H), 1.27-1.17 (m, 2H) |
| 333 | 828 | 829 | δ (DMSO-d6) 10.86 (s, 1H), 9.06-8.96 (m, 1H), 8.46 (d, J = 3.5 |
| | | | Hz, 1H), 8.05 (br d, J = 7.8 Hz, 1H), 7.99 (s, 1H), 7.91-7.85 (m, |
| | | | 1H), 7.74-7.67 (m, 2H), 7.63-7.49 (m, 3H), 7.30 (br d, J = 8.1 |
| | | | Hz, 1H), 6.96-6.89 (m, 2H), 6.52 (d, J = 9.3 Hz, 1H), 6.37 (dt, |
| | | | J = 1.0, 6.7 Hz, 1H), 4.27 (dd, J = 5.1, 9.3 Hz, 1H), 3.97 (br d, J = |
| | | | 11.9 Hz, 2H), 3.80-3.70 (m, 1H), 3.49 (br d, J = 10.6 Hz, 2H), |
| | | | 2.96 (br s, 4H), 2.83-2.74 (m, 2H), 2.69-2.59 (m, 2H), 2.36- |
| | | | 2.22 (m, 2H), 2.20-2.04 (m, 3H), 1.99-1.89 (m, 2H), 1.87-1.58 |
| | | | (m, 9H), 1.43-1.21 (m, 7H) |
| 334 | 827 | 828 | δ (DMSO-d6) 10.88 (s, 1H), 8.46 (d, J = 3.7 Hz, 1H), 8.18 (s, |
| | | | 1H), 8.05 (br d, J = 7.4 Hz, 1H), 7.99 (s, 1H), 7.75-7.71 (m, 2H), |
| | | | 7.70-7.67 (m, 1H), 7.62 (br d, J = 1.2 Hz, 1H), 7.60 (s, 1H), 7.55 |
| | | | (ddd, J = 9.1, 6.8, 2.0 Hz, 1H), 7.42 (s, 1H), 7.30 (br d, J = 8.1 |
| | | | Hz, 1H), 7.03 (d, J = 8.3 Hz, 1H), 6.52 (d, J = 9.3 Hz, 1H), 6.39- |
| | | | 6.34 (m, 1H), 4.33 (dd, J = 9.8, 5.1 Hz, 1H), 3.99-3.94 (m, 3H), |
| | | | 3.78-3.70 (m, 1H), 3.61-3.55 (m, 1H), 3.07 (br d, J = 8.8 Hz, |
| | | | 2H), 2.88 (br t, J = 5.3 Hz, 2H), 2.73-2.60 (m, 3H), 2.58 (br s, |
| | | | 1H), 2.34 (br dd, J = 9.0, 4.7 Hz, 1H), 2.30-2.22 (m, 1H), 2.21- |
| | | | 2.12 (m, 1H), 1.92 (br d, J = 11.3 Hz, 2H), 1.86 (br d, J = 11.4 |
| | | | Hz, 4H), 1.80-1.73 (m, 4H), 1.69-1.63 (m, 1H), 1.40 (s, 1H), |
| | | | 1.36-1.28 (m, 4H), 1.28-1.18 (m, 3H), 1.14 (br d, J = 6.4 Hz, |
| | | | 1H), 0.91 (q, J = 11.7 Hz, 2H) |
| 335 | 843 | 844 | δ (DMSO-d6) 11.13 (s, 1H), 8.49 (d, J = 3.2 Hz, 1H), 8.21 (s, |
| | | | 1H), 8.08 (br d, J = 6.1 Hz, 1H), 8.00 (br s, 1H), 7.75 (br d, J = |
| | | | 8.2 Hz, 1H), 7.72-7.69 (m, 1H), 7.63 (br d, J = 7.9 Hz, 1H), 7.58 |
| | | | (ddd, J = 9.1, 6.8, 2.0 Hz, 1H), 7.34 (br d, J = 7.3 Hz, 1H), 7.11 |
| | | | (br s, 1H), 7.04 (br d, J = 8.1 Hz, 1H), 6.93 (br d, J = 6.6 Hz, 1H), |
| | | | 6.55 (d, J = 9.2 Hz, 1H), 6.39 (t, J = 6.6 Hz, 1H), 5.37 (br dd, J = |
| | | | 12.7, 5.3 Hz, 1H), 4.44-4.33 (m, 1H), 4.00-3.91 (m, 1H), 3.87- |
| | | | 3.77 (m, 1H), 3.36 (s, 3H), 3.14 (br d, J = 9.2 Hz, 2H), 3.01- |
| | | | 2.87 (m, 2H), 2.83-2.68 (m, 3H), 2.67 (br s, 1H), 2.64-2.58 (m, |
| | | | 2H), 2.55-2.54 (m, 2H), 2.52-2.46 (m, 1H), 2.24 (br d, J = 6.1 |
| | | | Hz, 2H), 2.06-1.91 (m, 3H), 1.81 (br s, 6H), 1.72-1.60 (m, 2H), |
| | | | 1.54-1.38 (m, 4H), 1.36-1.19 (m, 2H), 0.85 (br s, 1H) |
| 336 | 807 | 808 | δ (DMSO-d6) 10.79 (s, 1H), 8.45 (d, J = 3.7 Hz, 1H), 8.14 (s, |
| | | | 0.5H), 8.03 (d, J = 7.6 Hz, 1H), 7.98 (s, 1H), 7.74-7.65 (m, 3H), |
| | | | 7.62-7.51 (m, 2H), 7.30 (br d, J = 8.1 Hz, 1H), 6.81 (t, J = 9.4 |
| | | | Hz, 1H), 6.54-6.45 (m, 2H), 6.44-6.33 (m, 2H), 5.79 (d, J = 7.8 |
| | | | Hz, 1H), 4.31-4.17 (m, 1H), 3.77-3.65 (m, 1H), 3.54-3.45 (m, |
| | | | 1H), 3.09 (br d, J = 10.6 Hz, 2H), 2.88-2.77 (m, 2H), 2.74-2.65 |
| | | | (m, 1H), 2.61-2.54 (m, 2H), 2.21 (br s, 3H), 2.12-1.98 (m, 3H), |
| | | | 1.95-1.84 (m, 3H), 1.78-1.53 (m, 8H), 1.43-1.32 (m, 3H), 1.30- |
| | | | 1.12 (m, 5H) |
| 337 | 806 | 807 | δ 10.80 (br s, 1H), 8.45 (br d, J = 1.9 Hz, 1H), 8.16 (s, 0.4H), 8.06- |
| | | | 8.02 (m, 1H), 7.98 (br s, 1H), 7.74-7.70 (m, 3H), 7.61-7.53 |
| | | | (m, 2H), 7.32 (br d, J = 6.4 Hz, 1H), 6.99-6.95 (m, 1H), 6.51 (br |
| | | | d, J = 9.0 Hz, 1H), 6.47-6.42 (m, 2H), 6.40-6.32 (m, 1H), 6.03 |
| | | | (br d, J = 7.5 Hz, 1H), 4.33-4.29 (m, 1H), 3.74-3.68 (m, 1H), |
| | | | 3.14-3.12 (m, 2H), 3.04-2.97 (m, 2H), 2.78-2.64 (m, 3H), 2.42- |
| | | | 2.31 (m, 3H), 2.27-2.22 (m, 1H), 2.09-2.05 (m, 1H), 1.94- |
| | | | 1.85 (m, 3H), 1.76 (br dd, J = 2.0, 4.3 Hz, 2H), 1.70-1.60 (m, |
| | | | 8H), 1.55-1.50 (m, 4H), 1.37-1.28 (m, 5H) |
| 338 | 860 | 861 | δ 11.08 (br s, 1H), 8.46 (br s, 1H), 7.98 (br s, 1H), 7.85-7.69 (m, |
| | | | 4H), 7.66-7.50 (m, 2H), 7.33 (t, J = 8.8 Hz, 2H), 7.10 (s, 1H), |
| | | | 7.05-6.96 (m, 1H), 6.92 (d, J = 8.1 Hz, 1H), 5.34 (dd, J = 5.4, |
| | | | 12.8 Hz, 1H), 4.41-4.20 (m, 1H), 4.09-3.83 (m, 2H), 3.33 (s, |
| | | | 3H), 3.22-3.07 (m, 1H), 3.02-2.84 (m, 3H), 2.79-2.54 (m, 4H), |
| | | | 2.37-2.26 (m, 2H), 2.04-1.85 (m, 5H), 1.82-1.60 (m, 8H), 1.53- |
| | | | 1.16 (m, 8H), 1.11-0.94 (m, 2H) |
| 339 | 846 | 847 | δ 11.21 (s, 1H), 9.80-9.62 (m, 1H), 8.58-8.43 (m, 2H), 8.08- |
| | | | 7.94 (m, 2H), 7.75-7.66 (m, 2H), 7.62-7.51 (m, 2H), 7.30-7.13 |
| | | | (m, 3H), 6.90-6.83 (m, 1H), 6.51 (d, J = 9.2 Hz, 1H), 6.36 (t, |
| | | | J = 6.5 Hz, 1H), 5.32 (br dd, J = 4.8, 13.0 Hz, 1H), 3.86 (br s, 4H), |
| | | | 3.64-3.58 (m, 3H), 3.53-3.46 (m, 2H), 3.20-3.08 (m, 4H), 3.06 |
| | | | (br s, 4H), 2.87 (br d, J = 12.7 Hz, 1H), 2.72-2.60 (m, 2H), 2.17- |
| | | | 2.06 (m, 2H), 2.03-1.92 (m, 5H), 1.87 (br s, 2H), 1.59-1.49 |
| | | | (m, 2H), 1.39-1.28 (m, 4H) |
| 340 | 832 | 833 | δ 11.20 (s, 1H), 9.50-9.29 (m, 2H), 8.51 (d, J = 3.5 Hz, 1H), 8.10- |
| | | | 7.96 (m, 2H), 7.76-7.66 (m, 2H), 7.63-7.45 (m, 3H), 7.25- |
| | | | 7.12 (m, 2H), 6.87 (br d, J = 8.3 Hz, 1H), 6.52 (d, J = 9.1 Hz, 1H), |
| | | | 6.36 (s, 1H), 5.36-5.22 (m, 1H), 4.39-4.21 (m, 4H), 3.83-3.76 |
| | | | (m, 6H), 3.64 (br d, J = 10.9 Hz, 4H), 3.20 (br s, 1H), 3.19-3.16 |
| | | | (m, 1H), 3.12 (br s, 1H), 3.08-2.97 (m, 3H), 2.96-2.92 (m, 1H), |
| | | | 2.89 (br s, 1H), 2.75-2.61 (m, 2H), 2.33 (br s, 1H), 2.18-2.06 |
| | | | (m, 2H), 1.97 (br d, J = 8.6 Hz, 4H), 1.62-1.47 (m, 2H), 1.43- |
| | | | 1.31 (m, 1H) |
| 341 | 808 | 809 | δ 10.81 (s, 1H), 9.75-9.32 (m, 2H), 8.51 (d, J = 3.6 Hz, 1H), 8.06 |
| | | | (br d, J = 7.5 Hz, 1H), 8.00 (s, 1H), 7.76-7.67 (m, 2H), 7.62 (dd, |
| | | | J = 0.9, 8.0 Hz, 1H), 7.56 (ddd, J = 2.1, 6.8, 9.1 Hz, 1H), 7.49 (br |
| | | | d, J = 7.1 Hz, 1H), 6.90 (t, J = 9.3 Hz, 1H), 6.65-6.41 (m, 3H), |
| | | | 6.37 (dt, J = 1.0, 6.7 Hz, 1H), 4.43-4.21 (m, 4H), 4.02 (br d, J = |
| | | | 4.4 Hz, 1H), 3.76-3.57 (m, 3H), 3.39-2.83 (m, 14H), 2.81-2.66 |
| | | | (m, 1H), 2.64-2.54 (m, 1H), 2.16-1.78 (m, 7H), 1.76-1.30 (m, |
| | | | 4H) |
| 342 | 831 | 832 | δ 11.21 (s, 1H), 9.63-9.37 (m, 1H), 8.47 (br s, 1H), 8.06 (br d, |
| | | | J = 6.9 Hz, 1H), 7.97 (br s, 1H), 7.79-7.65 (m, 2H), 7.65-7.50 |
| | | | (m, 2H), 7.32 (br d, J = 6.0 Hz, 1H), 7.25-7.12 (m, 2H), 6.87 (br |
| | | | d, J = 7.9 Hz, 1H), 6.53 (d, J = 9.1 Hz, 1H), 6.37 (br t, J = 6.3 Hz, |
| | | | 1H), 5.33 (br dd, J = 5.1, 13.0 Hz, 1H), 4.46-4.28 (m, 1H), 4.07- |
| | | | 3.90 (m, 1H), 3.79 (br s, 3H), 3.58 (br t, J = 10.1 Hz, 2H), 3.15 |
| | | | (br dd, J = 8.9, 19.9 Hz, 4H), 3.04-2.82 (m, 4H), 2.81-2.60 (m, |
| | | | 3H), 2.14 (td, J = 4.9, 10.3 Hz, 1H), 1.95 (br d, J = 10.0 Hz, 2H), |
| | | | 1.84-1.15 (m, 12H), 1.14-0.81 (m, 2H) |
| 343 | 807 | 808 | δ (DMSO-d6) 10.86-10.76 (m, 1H), 9.20-9.09 (m, 1H), 8.53- |
| | | | 8.47 (m, 1H), 8.06 (br d, J = 7.4 Hz, 1H), 7.99 (s, 1H), 7.75-7.68 |
| | | | (m, 2H), 7.64-7.60 (m, 1H), 7.58-7.52 (m, 1H), 7.47-7.40 (m, |
| | | | 1H), 6.99-6.92 (m, 1H), 6.55-6.45 (m, 3H), 6.36 (dt, J = 6.7, |
| | | | 1.1 Hz, 1H), 5.78-5.74 (m, 1H), 4.37-4.23 (m, 2H), 4.08-3.91 |
| | | | (m, 2H), 3.88-3.81 (m, 1H), 3.77-3.66 (m, 2H), 3.57-3.49 (m, |
| | | | 3H), 3.24-3.14 (m, 4H), 3.08-2.95 (m, 3H), 2.93-2.82 (m, 3H), |
| | | | 2.79-2.69 (m, 1H), 2.63-2.59 (m, 1H), 2.28 (br d, J = 9.6 Hz, |
| | | | 1H), 2.11-2.06 (m, 1H), 2.00 (s, 1H), 1.98-1.85 (m, 8H), 1.53- |
| | | | 1.43 (m, 1H), 1.40-1.31 (m, 1H), 1.20-1.13 (m, 1H) |
| 344 | 830 | 831 | δ (DMSO-d6) 11.08 (s, 1H), 8.47 (d, J = 3.6 Hz, 1H), 8.05 (br d, |
| | | | J = 8.1 Hz, 1H), 7.97 (br s, 1H), 7.93-7.83 (m, 1H), 7.76-7.66 |
| | | | (m, 2H), 7.63-7.52 (m, 2H), 7.25 (br d, J = 7.8 Hz, 1H), 7.02- |
| | | | 6.92 (m, 1H), 6.92-6.84 (m, 1H), 6.71-6.63 (m, 1H), 6.52 (d, |
| | | | J = 9.3 Hz, 1H), 6.36 (dt, J = 6.7, 1.2 Hz, 1H), 3.82-3.74 (m, 2H), |
| | | | 3.71-3.62 (m, 2H), 3.58-3.53 (m, 2H), 3.32 (br s, 3H), 3.07- |
| | | | 2.84 (m, 4H), 2.74-2.62 (m, 4H), 2.41-2.36 (m, 1H), 2.16-2.09 |
| | | | (m, 2H), 2.05-1.87 (m, 6H), 1.86-1.71 (m, 5H), 1.40-1.19 (m, |
| | | | 5H) |
| 345 | 792 | 793 | δ (DMSO-d6) 10.81 (s, 1H), 9.02-8.86 (m, 1H), 8.47 (d, J = 3.5 |
| | | | Hz, 1H), 8.05 (br d, J = 7.5 Hz, 1H), 7.98 (br s, 1H), 7.75-7.68 |
| | | | (m, 3H), 7.61 (br d, J = 7.9 Hz, 1H), 7.55 (ddd, J = 9.1, 6.8, 1.9 |
| | | | Hz, 1H), 7.30-7.18 (m, 1H), 6.95 (br t, J = 8.8 Hz, 1H), 6.55- |
| | | | 6.45 (m, 3H), 6.36 (dt, J = 0.9, 6.7 Hz, 1H), 4.32 (br dd, J = 11.4, |
| | | | 4.7 Hz, 1H), 3.65 (br d, J = 1.8 Hz, 2H), 3.49 (br d, J = 10.4 Hz, |
| | | | 3H), 3.23 (br s, 1H), 3.20-3.12 (m, 2H), 2.98-2.92 (m, 1H), |
| | | | 2.78-2.67 (m, 2H), 2.60 (br d, J = 4.0 Hz, 1H), 2.08 (br s, 4H), |
| | | | 1.96-1.90 (m, 4H), 1.87 (br d, J = 3.5 Hz, 3H), 1.79 (br d, J = |
| | | | 11.4 Hz, 2H), 1.46 (br t, J = 8.9 Hz, 4H), 1.35-1.23 (m, 4H) |
| 346 | 859 | 860 | δ (DMSO-d6) 10.58-10.53 (m, 1H), 8.49 (d, J = 3.6 Hz, 1H), |
| | | | 8.09-8.03 (m, 1H), 7.99 (s, 1H), 7.74-7.71 (m, 1H), 7.70-7.68 |
| | | | (m, 1H), 7.62-7.59 (m, 1H), 7.57-7.52 (m, 2H), 7.45-7.40 (m, |
| | | | 2H), 7.07-7.02 (m, 1H), 6.54-6.50 (m, 1H), 6.39-6.33 (m, 1H), |
| | | | 5.69 (s, 1H), 3.97 (s, 3H), 3.93-3.89 (m, 3H), 3.24 (br d, J = 12.8 |
| | | | Hz, 1H), 3.15-3.04 (m, 3H), 2.99-2.91 (m, 3H), 2.78-2.74 (m, |
| | | | 2H), 2.19-2.08 (m, 3H), 2.01-1.89 (m, 4H), 1.86-1.66 (m, |
| | | | 10H), 1.50-1.37 (m, 3H) |
| 347 | 846 | 847 | δ (DMSO-d6) 11.18 (br d, J = 3.1 Hz, 1H), 8.46 (d, J = 3.6 Hz, |
| | | | 1H), 8.06 (br d, J = 7.5 Hz, 1H), 7.98 (br s, 1H), 7.73-7.67 (m, |
| | | | 2H), 7.60 (br d, J = 8.0 Hz, 1H), 7.57-7.53 (m, 1H), 7.30 (br s, |
| | | | 2H), 7.20-7.14 (m, 1H), 7.12-7.06 (m, 1H), 6.52 (d, J = 9.3 Hz, |
| | | | 1H), 6.36 (t, J = 6.7 Hz, 1H), 5.34 (br dd, J = 12.9 , 5.4 Hz, 1H), |
| | | | 5.19 (br s, 1H), 4.08-3.99 (m, 1H), 3.83-3.76 (m, 1H), 3.67 (br |
| | | | d, J = 11.4 Hz, 1H), 3.04-2.95 (m, 3H), 2.92-2.83 (m, 2H), 2.77- |
| | | | 2.66 (m, 4H), 2.17-2.12 (m, 1H), 1.98-1.90 (m, 4H), 1.76 (br |
| | | | d, J = 9.4 Hz, 4H), 1.61 (br d, J = 12.0 Hz, 6H), 1.49 (br d, J = |
| | | | 14.0 Hz, 2H), 1.39 (br d, J = 15.5 Hz, 2H), 1.33-1.23 (m, 4H) |
| 348 | 822 | 823 | δ (DMSO-d6) 10.80 (br s, 1H), 8.46 (d, J = 3.5 Hz, 1H), 8.07 (br |
| | | | d, J = 7.3 Hz, 1H), 7.98 (br s, 1H), 7.75-7.67 (m, 2H), 7.62 (s, |
| | | | 1H), 7.55 (ddd, J = 9.1, 6.8, 1.9Hz, 1H), 7.30 (br d, J = 7.9 Hz, |
| | | | 1H), 7.02-6.93 (m, 1H), 6.52 (d, J = 9.3 Hz, 1H), 6.45 (br d, J = |
| | | | 6.0 Hz, 1H), 6.36 (t, J = 6.6 Hz, 1H), 6.01 (br d, J = 7.8 Hz, 1H), |
| | | | 5.36-5.19 (m, 1H), 4.37-4.27 (m, 1H), 4.10-3.95 (m, 1H), 3.85- |
| | | | 3.74 (m, 1H), 3.72-3.60 (m, 1H), 3.04-2.91 (m, 3H), 2.74 |
| | | | (ddd, J = 5.3, 12.1, 17.4 Hz, 3H), 2.59 (br d, J = 3.9 Hz, 2H), 2.08 |
| | | | (td, J = 4.1, 8.6 Hz, 1H), 2.04-1.81 (m, 6H), 1.76 (br d, J = 12.8 |
| | | | Hz, 1H), 1.73-1.53 (m, 8H), 1.53-1.44 (m, 3H), 1.39 (br d, J = |
| | | | 14.3 Hz, 2H), 1.34-1.21 (m, 4H) |
| 349 | 830 | 831 | δ (DMSO-d6) 11.22 (s, 1H), 9.10-9.03 (m, 1H), 8.47 (d, J = 3.6 |
| | | | Hz, 1H), 8.05 (br d, J = 7.7 Hz, 1H), 7.99 (s, 1H), 7.79 (br t, J = |
| | | | 5.0 Hz, 1H), 7.73 (br d, J = 4.3 Hz, 1H), 7.71-7.68 (m, 1H), 7.63- |
| | | | 7.59 (m, 1H), 7.56 (ddd, J = 9.1, 6.7, 2.0 Hz, 1H), 7.33-7.27 |
| | | | (m, 2H), 7.23 (d, J = 8.0 Hz, 1H), 7.10-7.00 (m, 1H), 6.52 (d, |
| | | | J = 9.2 Hz, 1H), 6.37 (dt, J = 6.7, 1.1 Hz, 1H), 5.37 (dd, J = 12.9, |
| | | | 5.1 Hz, 1H), 3.78-3.69 (m, 1H), 3.52-3.47 (m, 2H), 3.20-3.06 |
| | | | (m, 3H), 2.94-2.86 (m, 4H), 2.71-2.61 (m, 2H), 2.30-2.22 (m, |
| | | | 1H), 2.15 (td, J = 9.7, 5.0 Hz, 1H), 2.06-2.01 (m, 3H), 1.97- |
| | | | 1.87 (m, 4H), 1.84-1.78 (m, 3H), 1.67 (br d, J = 8.7 Hz, 1H), |
| | | | 1.41 (br s, 6H), 1.27-1.16 (m, 2H), 1.03-0.93 (m, 2H) |
| 350 | 830 | 831 | δ (DMSO-d6) 11.21 (br d, J = 3.6 Hz, 1H), 8.49-8.43 (m, 1H), |
| | | | 8.16 (d, J = 4.3 Hz, 1H), 8.09-8.03 (m, 1H), 7.98 (br s, 1H), 7.76- |
| | | | 7.67 (m, 2H), 7.60 (br dd, J = 1.8, 6.8 Hz, 1H), 7.58-7.52 (m, |
| | | | 1H), 7.33-7.26 (m, 2H), 7.17 (br dd, J = 4.1, 7.8 Hz, 1H), 7.10 |
| | | | (br d, J = 1.9 Hz, 1H), 6.52 (br dd, J = 3.8, 9.1 Hz, 1H), 6.36 (br |
| | | | d, J = 3.9 Hz, 1H), 5.40-5.31 (m, 1H), 4.36 (dt, J = 1.8, 5.8 Hz, |
| | | | 1H), 3.96-3.88 (m, 1H), 3.80 (br dd, J = 6.6, 11.7 Hz, 1H), 3.06- |
| | | | 3.00 (m, 2H), 2.96-2.85 (m, 3H), 2.74-2.66 (m, 4H), 2.19- |
| | | | 2.05 (m, 3H), 1.97-1.91 (m, 2H), 1.81-1.72 (m, 4H), 1.70-1.56 |
| | | | (m, 5H), 1.49-1.16 (m, 8H), 1.09-0.85 (m, 2H) |
| 351 | 827 | 828 | δ (DMSO-d6) 10.90 (s, 1H), 9.23-9.04 (m, 1H), 8.46 (br s, 1H), |
| | | | 8.06 (br d, J = 6.0 Hz, 1H), 7.97 (br s, 1H), 7.77-7.65 (m, 3H), |
| | | | 7.62 (br d, J = 6.4 Hz, 1H), 7.59-7.54 (m, 1H), 7.41 (br s, 1H), |
| | | | 7.30 (br s, 1H), 7.03 (br d, J = 7.1 Hz, 1H), 6.53 (br d, J = 9.3 Hz, |
| | | | 1H), 6.37 (br s, 1H), 4.43-4.31 (m, 2H), 3.95-3.92 (m, 1H), |
| | | | 3.99 (s, 3H), 3.86-3.74 (m, 1H), 3.68-3.56 (m, 2H), 3.17-2.90 |
| | | | (m, 6H), 2.81-2.70 (m, 2H), 2.69-2.59 (m, 2H), 2.41-2.31 (m, |
| | | | 2H), 2.17 (br dd, J = 12.8 Hz, 5.2, 2H), 2.08 (br s, 2H), 1.94 (br |
| | | | s, 2H), 1.80-1.75 (m, 1H), 1.72-1.53 (m, 6H), 1.42 (br d, J = |
| | | | 3.3 Hz, 2H), 1.26 (br dd, J = 12.4 Hz, 6.4, 2H), 1.05-0.92 (m, |
| | | | 2H) |
| 352 | 807 | 808 | δ 8.46-8.46 (br d, J = 1.5 Hz, 1H), 8.26-8.25 (d, J = 3.8 Hz, 1H), |
| | | | 8.18-8.16 (br d, J = 7.4 Hz, 1H), 7.98-7.96 (m, 1H), 7.68-7.64 |
| | | | (t, J = 8.0 Hz, 1H), 7.51-7.47 (m, 3H), 6.95-6.90 (t, J = 9.0 Hz, |
| | | | 1H), 6.69-6.66 (d, J = 9.4 Hz, 1H), 6.46-6.40 (m, 2H), 6.38-6.34 |
| | | | (t, J = 6.6 Hz, 1H), 5.12-5.10 (br d, J = 6.9 Hz, 1H), 4.68-4.67 (br |
| | | | d, J = 2.3 Hz, 1H), 4.05-4.00 (br dd, J = 12.8, 4.8 Hz, 1H), 3.87- |
| | | | 3.80 (m, 1H), 3.40-3.33 (m, 3H), 3.27-3.16 (m, 3H), 3.10- |
| | | | 3.07 (br t, J = 5.6 Hz, 2H), 2.94-2.88 (m, 2H), 2.82-2.77 (m, |
| | | | 1H), 2.57-2.46 (m, 2H), 2.34-2.16 (m, 4H), 2.10-2.03 (m, 2H), |
| | | | 1.97-1.83 (m, 7H), 1.63-1.57 (m, 2H), 1.51-1.43 (m, 2H), 1.36- |
| | | | 1.26 (m, 2H), 1.08-0.98 (m, 2H) |
| 353 | 831 | 832 | δ (DMSO-d6) 11.23-11.18 (m, 1H), 8.49 (d, J = 3.5 Hz, 1H), |
| | | | 8.21-8.18 (m, 1H), 8.06 (br d, J = 8.0 Hz, 1H), 8.01-7.97 (m, |
| | | | 1H), 7.75-7.66 (m, 2H), 7.63-7.58 (m, 1H), 7.58-7.51 (m, 1H), |
| | | | 7.39 (br d, J = 7.9 Hz, 1H), 7.30 (s, 1H), 7.19-7.14 (m, 1H), 7.12- |
| | | | 7.06 (m, 1H), 6.54-6.49 (m, 1H), 6.39-6.33 (m, 1H), 5.38- |
| | | | 5.31 (m, 1H), 4.31-4.23 (m, 1H), 3.98-3.83 (m, 2H), 3.20-3.10 |
| | | | (m, 2H), 3.02-2.84 (m, 6H), 2.82-2.61 (m, 4H), 2.34-2.19 (m, |
| | | | 4H), 2.17-2.12 (m, 1H), 1.94 (br t, J = 10.7 Hz, 4H), 1.87 (br s, |
| | | | 1H), 1.74 (br s, 4H), 1.68-1.58 (m, 2H), 1.49-1.40 (m, 3H), |
| | | | 1.36-1.28 (m, 1H) |
| 354 | 844 | 845 | δ (DMSO-d6) 11.21 (br s, 1H), 8.46 (d, J = 3.5 Hz, 1H), 8.17 (s, |
| | | | 1H), 8.04 (br d, J = 6.8 Hz, 1H), 7.97 (br s, 1H), 7.72-7.65 (m, |
| | | | 1H), 7.60 (br d, J = 7.9 Hz, 1H), 7.53 (br s, 1H), 7.43 (dd, J = 9.4, |
| | | | 2.4 Hz, 1H), 7.29 (br d, J = 11.0 Hz, 2H), 7.20-7.15 (m, 1H), |
| | | | 7.09 (br d, J = 6.8 Hz, 1H), 6.47 (d, J = 9.3 Hz, 1H), 5.40-5.31 |
| | | | (m, 1H), 4.41-4.29 (m, 1H), 3.98-3.88 (m, 1H), 3.86-3.75 (m, |
| | | | 1H), 3.10-3.00 (m, 2H), 2.97-2.82 (m, 2H), 2.80-2.59 (m, 4H), |
| | | | 2.47-2.35 (m, 3H), 2.21-2.08 (m, 3H), 2.07 (s, 3H), 1.94 (br d, |
| | | | J = 9.9 Hz, 2H), 1.76 (br d, J = 8.7 Hz, 3H), 1.70-1.57 (m, 4H), |
| | | | 1.54-1.14 (m, 8H), 1.07-0.85 (m, 2H) |
| 355 | 836 | 837 | δ (DMSO-d6) 11.21 (s, 1H), 8.43 (d, J = 3.3 Hz, 1H), 8.15 (s, |
| | | | 1H), 7.96 (br s, 1H), 7.85 (br s, 1H), 7.57-7.54 (m, 2H), 7.31 (br |
| | | | s, 1H), 7.26 (br d, J = 7.5 Hz, 1H), 7.20-7.17 (m, 1H), 7.10 (br |
| | | | d, J = 7.9 Hz, 1H), 5.40-5.31 (m, 1H), 4.38 (br t, J = 5.3 Hz, 3H), |
| | | | 4.01-3.90 (m, 1H), 3.85-3.76 (m, 1H), 3.72 (br t, J = 6.0 Hz, |
| | | | 2H), 3.21-3.12 (m, 2H), 3.04-2.84 (m, 3H), 2.81-2.71 (m, 1H), |
| | | | 2.69-2.59 (m, 4H), 2.32-2.25 (m, 1H), 2.17-2.11 (m, 3H), 1.98- |
| | | | 1.89 (m, 2H), 1.86-1.78 (m, 3H), 1.77-1.58 (m, 6H), 1.53- |
| | | | 1.37 (m, 5H), 1.36-1.19 (m, 3H), 1.07-0.89 (m, 2H) |
| 356 | 834 | 835 | δ (DMSO-d6) 11.22 (s, 1H), 8.43 (d, J = 3.6 Hz, 1H), 7.87 (br s, |
| | | | 2H), 7.58-7.52 (m, 1H), 7.46 (br d, J = 8.1 Hz, 1H), 7.31 (br s, |
| | | | 1H), 7.27 (br d, J = 7.6 Hz, 1H), 7.19-7.15 (m, 1H), 7.10 (br d, |
| | | | J = 7.4 Hz, 1H), 5.35 (br dd, J = 5.4, 13.1 Hz, 1H), 4.37 (td, J = |
| | | | 1.9, 8.2 Hz, 1H), 4.00-3.90 (m, 1H), 3.85-3.75 (m, 1H), 3.68- |
| | | | 3.61 (m, 2H), 3.08-2.96 (m, 3H), 2.91-2.84 (m, 1H), 2.78-2.72 |
| | | | (m, 1H), 2.71-2.65 (m, 2H), 2.45-2.39 (m, 4H), 2.14 (br s, 3H), |
| | | | 1.94-1.82 (m, 7H), 1.81-1.72 (m, 4H), 1.71-1.58 (m, 5H), 1.47- |
| | | | 1.36 (m, 4H), 1.34-1.20 (m, 3H), 1.05-0.84 (m, 2H) |
| 357 | 898 | 899 | δ (DMSO-d6) 11.25-11.17 (m, 1H), 8.47 (d, J = 3.4 Hz, 1H), |
| | | | 8.37 (br s, 1H), 8.15 (s, 1H), 8.09 (br d, J = 6.0 Hz, 1H), 8.03 (br |
| | | | s, 1H), 7.81 (dd, J = 9.7, 2.7 Hz, 1H), 7.74-7.65 (m, 2H), 7.32- |
| | | | 7.26 (m, 2H), 7.20-7.16 (m, 1H), 7.12-7.05 (m, 1H), 6.71-6.67 |
| | | | (m, 1H), 5.35 (dd, J = 12.9, 5.3 Hz, 1H), 4.42-4.29 (m, 1H), 3.92 |
| | | | (br s, 1H), 3.85-3.74 (m, 1H), 3.21-3.05 (m, 3H), 2.94-2.84 |
| | | | (m, 2H), 2.73-2.60 (m, 5H), 2.26-2.13 (m, 2H), 2.00-1.87 (m, |
| | | | 3H), 1.86-1.73 (m, 4H), 1.70-1.57 (m, 4H), 1.49-1.33 (m, 5H), |
| | | | 1.32-1.15 (m, 3H), 1.07-0.83 (m, 2H) |
| 358 | 845 | 846 | δ 11.17 (br s, 1H), 8.45 (d, J = 3.6 Hz, 1H), 8.04 (br d, J = 7.5 Hz, |
| | | | 1H), 7.97 (s, 1H), 7.78-7.64 (m, 3H), 7.63-7.49 (m, 2H), 7.21 |
| | | | (d, J = 7.6 Hz, 1H), 7.12-7.00 (m, 2H), 6.76 (dd, J = 1.9, 8.7 Hz, |
| | | | 1H), 6.51 (d, J = 8.9 Hz, 1H), 6.40-6.31 (m, 1H), 5.28 (dd, J = |
| | | | 5.3, 12.9 Hz, 1H), 3.63-3.55 (m, 2H), 3.53-3.47 (m, 1H), 2.94- |
| | | | 2.84 (m, 1H), 2.82-2.74 (m, 2H), 2.68-2.59 (m, 4H), 2.33 (br |
| | | | d, J = 1.8 Hz, 1H), 2.11 (br d, J = 7.4 Hz, 3H), 1.95 (br d, J = 6.5 |
| | | | Hz, 4H), 1.87-1.70 (m, 6H), 1.67-1.53 (m, 4H), 1.39-1.10 (m, |
| | | | 8H) |
| 359 | 827 | 828 | δ 11.20 (br s, 1H), 8.22 (br s, 1H), 8.16 (s, 1H), 7.42 (br d, J = 8.6 |
| | | | Hz, 1H), 7.30 (s, 1H), 7.19-7.14 (m, 1H), 7.09 (br d, J = 8.0 Hz, |
| | | | 1H), 6.99 (br d, J = 6.4 Hz, 1H), 5.34 (br dd, J = 4.6, 12.8 Hz, |
| | | | 1H), 4.48-4.21 (m, 3H), 2.92 (br d, J = 9.6 Hz, 3H), 2.85 (br s, |
| | | | 3H), 2.77 (br d, J = 10.1 Hz, 3H), 2.71-2.61 (m, 3H), 2.16 (br d, |
| | | | J = 6.6 Hz, 3H), 2.06-1.87 (m, 9H), 1.80-1.62 (m, 9H), 1.55- |
| | | | 1.42 (m, 2H), 1.39-1.28 (m, 4H), 1.27-1.09 (m, 4H), 0.77-0.59 |
| | | | (m, 4H) |
| 360 | 846 | 847 | δ 11.19 (s, 1H), 8.46 (d, J = 3.4 Hz, 1H), 8.15 (s, 0.5H), 8.04 (br |
| | | | d, J = 7.4 Hz, 1H), 7.96 (br s, 1H), 7.76-7.65 (m, 2H), 7.60 (br |
| | | | d, J = 8.1 Hz, 1H), 7.57-7.51 (m, 1H), 7.47 (br d, J = 8.4 Hz, |
| | | | 1H), 7.24 (br d, J = 8.0 Hz, 1H), 7.10-7.02 (m, 2H), 6.77 (br d, |
| | | | J = 8.8 Hz, 1H), 6.51 (d, J = 9.3 Hz, 1H), 6.35 (t, J = 6.6 Hz, 1H), |
| | | | 5.29 (br dd, J = 5.1, 12.7 Hz, 1H), 2.94-2.81 (m, 4H), 2.71-2.56 |
| | | | (m, 6H), 2.47-2.32 (m, 7H), 2.23-2.05 (m, 4H), 1.96 (br s, 2H), |
| | | | 1.76 (br d, J = 7.0 Hz, 4H), 1.69-1.56 (m, 1H), 1.42-1.28 (m, |
| | | | 4H), 1.20 (br d, J = 9.4 Hz, 3H) |
| 361 | 807 | 808 | δ 10.77 (s, 1H), 8.45 (d, J = 3.6 Hz, 1H), 8.15 (s, 0.3H), 8.04 (d, |
| | | | J = 7.4 Hz, 1H), 7.98 (s, 1H), 7.76 (br t, J = 5.3 Hz, 1H), 7.73- |
| | | | 7.70 (m, 1H), 7.70-7.66 (m, 1H), 7.60 (br d, J = 7.8 Hz, 1H), |
| | | | 7.54 (ddd, J = 9.1, 6.7, 2.1 Hz, 1H), 7.29 (br d, J = 8.0 Hz, 1H), |
| | | | 6.82 (t, J = 9.3 Hz, 1H), 6.54-6.45 (m, 2H), 6.42-6.33 (m, 2H), |
| | | | 5.80 (d, J = 7.6 Hz, 1H), 4.24 (ddd, J = 11.7, 7.3, 4.9 Hz, 1H), |
| | | | 3.78-3.67 (m, 1H), 3.16 (br d, J = 11.4 Hz, 3H), 3.01 (br d, J = |
| | | | 7.4 Hz, 2H), 2.92 (br t, J = 5.9 Hz, 2H), 2.77-2.66 (m, 1H), 2.58 |
| | | | (br d, J = 4.0 Hz, 1H), 2.54 (br s, 2H), 2.38-2.18 (m, 3H), 2.07 |
| | | | (td, J = 4.2, 8.5 Hz, 1H), 1.98-1.88 (m, 2H), 1.87-1.82 (m, 2H), |
| | | | 1.81-1.75 (m, 2H), 1.66 (br s, 2H), 1.63 (br s, 2H), 1.60-1.54 |
| | | | (m, 1H), 1.47-1.33 (m, 2H), 1.32-1.22 (m, 2H), 1.21-1.08 (m, |
| | | | 3H) |
| 362 | 851 | 852 | δ (DMSO-d6) 11.23 (s, 1H), 8.44 (d, J = 3.6 Hz, 1H), 8.15 (s, |
| | | | 1H), 7.96 (s, 1H), 7.84 (br d, J = 5.5 Hz, 1H), 7.59-7.46 (m, 3H), |
| | | | 7.32 (s, 1H), 7.23 (br d, J = 7.6 Hz, 1H), 7.20-7.15 (m, 1H), 7.13- |
| | | | 7.07 (m, 1H), 5.35 (br dd, J = 12.8 Hz,5.1, 1H), 4.38 (t, J = 5.3 |
| | | | Hz, 2H), 3.74-3.55 (m, 5H), 2.97 (br d, J = 9.9 Hz, 2H), 2.90 (br |
| | | | s, 3H), 2.81 (br d, J = 10.0 Hz, 2H), 2.71-2.65 (m, 2H), 2.22 (br |
| | | | d, J = 6.1 Hz, 2H), 2.13 (td, J = 10.9 Hz, 5.6, 4H), 2.05 (br d, J = |
| | | | 10.6 Hz, 3H), 1.98 (br s, 2H), 1.78 (br s, 2H), 1.75-1.65 (m, 5H), |
| | | | 1.58-1.48 (m, 1H), 1.41-1.32 (m, 4H), 1.19 (q, J = 11.2 Hz, |
| | | | 2H) |
| 363 | 837 | 838 | δ (DMSO-d6) 11.23-11.21 (m, 1H), 8.48 (d, J = 3.4 Hz, 1H), |
| | | | 8.05 (s, 1H), 8.02 (s, 1H), 7.96 (br d, J = 6.5 Hz, 1H), 7.77-7.73 |
| | | | (m, 1H), 7.73-7.69 (m, 1H), 7.65-7.61 (m, 1H), 7.60-7.55 (m, |
| | | | 1H), 7.35 (br d, J = 8.2 Hz, 1H), 7.14-7.08 (m, 2H), 6.83 (s, 1H), |
| | | | 6.56 (s, 1H), 6.41-6.35 (m, 1H), 5.36-5.28 (m, 1H), 3.69-3.65 |
| | | | (m, 3H), 3.56-3.51 (m, 2H), 3.04-2.98 (m, 3H), 2.92-2.85 (m, |
| | | | 1H), 2.70-2.68 (m, 2H), 2.31-2.10 (m, 3H), 2.00-1.89 (m, 6H), |
| | | | 1.81 (br dd, J = 3.2, 11.4 Hz, 3H), 1.71-1.62 (m, 3H), 1.43-1.19 |
| | | | (m, 8H) |
| 364 | 807 | 808 | δ (DMSO-d6) 11.89-11.82 (m, 1H), 10.82-10.76 (m, 1H), 8.48- |
| | | | 8.43 (m, 1H), 8.39-8.32 (m, 1H), 7.93-7.84 (m, 2H), 7.74- |
| | | | 7.69 (m, 1H), 7.56 (t, J = 7.6 Hz, 1H), 7.47-7.41 (m, 1H), 7.38- |
| | | | 7.32 (m, 1H), 7.02-6.94 (m, 1H), 6.49-6.41 (m, 2H), 6.37-6.30 |
| | | | (m, 1H), 6.05-5.96 (m, 1H), 4.34-4.22 (m, 2H), 4.03-3.94 (m, |
| | | | 1H), 3.20-3.09 (m, 2H), 2.95-2.86 (m, 3H), 2.82-2.68 (m, 5H), |
| | | | 2.59 (br d, J = 4.9 Hz, 3H), 2.17-2.04 (m, 3H), 2.01-1.82 (m, |
| | | | 7H), 1.78-1.68 (m, 2H), 1.68-1.58 (m, 4H), 1.56-1.43 (m, 2H), |
| | | | 1.39-1.29 (m, 1H), 1.19-1.04 (m, 1H) |
| 365 | 821 | 822 | δ (DMSO-d6) 11.85 (s, 1H), 10.82 (s, 1H), 8.44 (d, J = 3.5 Hz, |
| | | | 1H), 8.30 (s, 1H), 7.93-7.86 (m, 2H), 7.72 (dd, J = 7.0, 1.7 Hz, |
| | | | 1H), 7.56 (t, J = 7.8 Hz, 1H), 7.45 (br d, J = 6.5 Hz, 1H), 7.20 (br |
| | | | d, J = 7.9 Hz, 1H), 7.02-6.93 (m, 1H), 6.52-6.46 (m, 2H), 6.46- |
| | | | 6.41 (m, 1H), 6.34 (t, J = 6.7 Hz, 1H), 6.19-5.96 (m, 1H), 4.38- |
| | | | 4.27 (m, 1H), 3.68 (br dd, J = 6.5, 3.2 Hz, 1H), 3.63-3.56 (m, |
| | | | 1H), 3.08-2.83 (m, 6H), 2.75 (ddd, J = 17.7, 11.9, 5.7 Hz, 3H), |
| | | | 2.60 (br d, J = 3.9 Hz, 1H), 2.58-2.54 (m, 1H), 2.21-2.04 (m, |
| | | | 4H), 2.03-1.96 (m, 3H), 1.89 (br dd, J = 11.7, 7.6 Hz, 2H), 1.84- |
| | | | 1.79 (m, 3H), 1.77-1.62 (m, 5H), 1.42-1.33 (m, 4H), 1.32- |
| | | | 1.23 (m, 2H) |
| 366 | 828 | 829 | δ 11.19 (br s, 1H), 8.50 (d, J = 3.5 Hz, 1H), 8.26 (s, 1H), 7.97 (br |
| | | | d, J = 7.4 Hz, 1H), 7.85 (br d, J = 7.5 Hz, 1H), 7.71 (br d, J = 7.6 |
| | | | Hz, 2H), 7.68-7.60 (m, 1H), 7.55-7.46 (m, 2H), 7.44-7.36 (m, |
| | | | 2H), 7.30 (s, 1H), 7.20-7.13 (m, 1H), 7.09 (br d, J = 8.1 Hz, 1H), |
| | | | 5.34 (br dd, J = 5.3, 12.9 Hz, 1H), 4.39-4.27 (m, 1H), 4.26-4.00 |
| | | | (m, 3H), 3.79 (br d, J = 13.0 Hz, 1H), 3.09-2.79 (m, 5H), 2.76- |
| | | | 2.54 (m, 5H), 2.37 (br dd, J = 9.6, 17.0 Hz, 1H), 2.15 (br d, J = |
| | | | 4.9 Hz, 4H), 1.96 (br t, J = 10.9 Hz, 2H), 1.88-1.53 (m, 9H), |
| | | | 1.14-0.83 (m, 2H) |
| 367 | 845 | 846 | δ 11.20 (br s, 1H), 8.52 (d, J = 3.6 Hz, 1H), 8.34 (s, 0.2H), 8.07 |
| | | | (d, J = 7.5 Hz, 1H), 7.99 (s, 1H), 7.75-7.66 (m, 2H), 7.64-7.51 |
| | | | (m, 2H), 7.50-7.41 (m, 1H), 7.34-7.26 (m, 1H), 7.24-7.15 (m, |
| | | | 1H), 7.11 (d, J = 8.6 Hz, 1H), 6.52 (d, J = 9.1 Hz, 1H), 6.36 (t, |
| | | | J = 6.7 Hz, 1H), 6.16 (br s, 1H), 5.36 (qd, J = 5.2, 12.8 Hz, 1H), |
| | | | 4.32 (br d, J = 12.6 Hz, 1H), 4.27-4.05 (m, 3H), 3.80 (br d, J = |
| | | | 12.3 Hz, 1H), 3.11-2.81 (m, 5H), 2.79-2.54 (m, 6H), 2.44-2.23 |
| | | | (m, 3H), 2.22-2.06 (m, 4H), 1.97 (br t, J = 10.6 Hz, 1H), 1.87- |
| | | | 1.53 (m, 7H), 1.14-0.87 (m, 2H) |
| 368 | 830 | 831 | δ 11.96-11.71 (m, 1H), 11.22 (s, 1H), 9.20-8.55 (m, 1H), 8.43 |
| | | | (d, J = 3.6 Hz, 1H), 8.36-8.28 (m, 1H), 7.96 (s, 2H), 7.75-7.68 |
| | | | (m, 1H), 7.59-7.51 (m, 1H), 7.46-7.40 (m, 1H), 7.30 (s, 1H), |
| | | | 7.22 (d, J = 8.1 Hz, 1H), 7.09 (d, J = 7.9 Hz, 1H), 6.32 (t, J = 6.7 |
| | | | Hz, 1H), 5.47-5.23 (m, 1H), 4.47-4.32 (m, 1H), 4.06-3.87 (m, |
| | | | 1H), 3.70-3.57 (m, 4H), 3.20-3.09 (m, 2H), 3.09-2.97 (m, 3H), |
| | | | 2.93-2.84 (m, 2H), 2.17-1.96 (m, 6H), 1.88-1.79 (m, 2H), 1.78- |
| | | | 1.56 (m, 8H), 1.51-1.30 (m, 4H), 1.19-0.88 (m, 3H) |
| 369 | 861 | 862 | δ 11.20 (br s, 1H), 8.44 (br s, 1H), 8.19 (s, 1H), 7.86 (d, J = 7.9 |
| | | | Hz, 1H), 7.80-7.62 (m, 3H), 7.61-7.45 (m, 3H), 7.42 (br d, J = |
| | | | 8.1 Hz, 1H), 7.31 (s, 1H), 7.22-7.14 (m, 1H), 7.10 (d, J = 7.8 |
| | | | Hz, 1H), 6.51 (d, J = 9.1 Hz, 1H), 6.35 (t, J = 6.7 Hz, 1H), 5.35 |
| | | | (dd, J = 5.2, 12.7 Hz, 1H), 3.76-3.49 (m, 3H), 2.97-2.82 (m, |
| | | | 5H), 2.81-2.72 (m, 2H), 2.71-2.61 (m, 2H), 2.23-2.10 (m, 3H), |
| | | | 2.07-1.88 (m, 6H), 1.83-1.58 (m, 8H), 1.56-1.43 (m, 1H), 1.42- |
| | | | 1.28 (m, 4H), 1.28-1.10 (m, 2H) |
| 370 | 846 | 847 | δ 11.20 (s, 1H), 8.48 (d, J = 2.8 Hz, 1H), 8.06 (br d, J = 7.8 Hz, |
| | | | 1H), 7.99 (s, 1H), 7.78-7.66 (m, 2H), 7.65-7.50 (m, 2H), 7.49- |
| | | | 7.35 (m, 1H), 7.30 (s, 1H), 7.21-7.04 (m, 2H), 6.51 (br d, J = |
| | | | 9.5 Hz, 1H), 6.35 (t, J = 6.5 Hz, 1H), 5.46-5.24 (m, 1H), 4.50- |
| | | | 4.30 (m, 1H), 4.20-3.88 (m, 2H), 3.76-3.53 (m, 1H), 3.26-2.81 |
| | | | (m, 5H), 2.76-2.62 (m, 4H), 2.37-2.21 (m, 2H), 2.19-2.05 (m, |
| | | | 4H), 1.93 (br d, J = 8.9 Hz, 4H), 1.78-1.56 (m, 6H), 1.54-1.32 |
| | | | (m, 2H), 1.29-0.83 (m, 6H) |
| 371 | 837 | 838 | δ 11.19 (br s, 1H), 8.45 (d, J = 3.5 Hz, 1H), 8.18 (br s, 1H), 7.97 |
| | | | (s, 1H), 7.85 (br d, J = 6.1 Hz, 1H), 7.61-7.49 (m, 2H), 7.36 (br |
| | | | d, J = 7.5 Hz, 1H), 7.30 (s, 1H), 7.21-7.13 (m, 1H), 7.12-7.04 |
| | | | (m, 1H), 5.34 (br dd, J = 5.2, 12.9 Hz, 1H), 4.37 (t, J = 5.3 Hz, |
| | | | 2H), 4.26 (br d, J = 13.0 Hz, 1H), 4.07 (br d, J = 12.9 Hz, 1H), |
| | | | 4.01-3.87 (m, 2H), 3.71 (br t, J = 6.0 Hz, 3H), 3.03 (br s, 2H), |
| | | | 2.93 (br d, J = 8.6 Hz, 3H), 2.82 (br d, J = 11.3 Hz, 3H), 2.70- |
| | | | 2.62 (m, 2H), 2.23-2.08 (m, 5H), 2.07-1.85 (m, 6H), 1.84-1.57 |
| | | | (m, 6H), 1.50 (br d, J = 8.4 Hz, 2H), 1.39-1.26 (m, 1H), 1.23- |
| | | | 1.01 (m, 2H) |
| 372 | 823 | 824 | δ 11.20 (s, 1H), 8.46 (d, J = 3.5 Hz, 1H), 7.80-7.61 (m, 2H), 7.59- |
| | | | 7.52 (m, 1H), 7.42-7.34 (m, 1H), 7.30 (s, 1H), 7.23-7.15 (m, |
| | | | 1H), 7.12-7.07 (m, 1H), 5.45-5.25 (m, 1H), 4.47 (t, J = 8.0 Hz, |
| | | | 2H), 4.32-4.25 (m, 1H), 4.16-4.07 (m, 3H), 4.05-3.89 (m, 2H), |
| | | | 3.00-2.76 (m, 8H), 2.33 (br s, 1H), 2.24-2.09 (m, 4H), 2.04- |
| | | | 1.87 (m, 6H), 1.84-1.63 (m, 7H), 1.57-1.43 (m, 2H), 1.40-1.28 |
| | | | (m, 2H), 1.25-1.02 (m, 2H) |
| 373 | 847 | 848 | δ 11.21 (d, J = 3.6 Hz, 1H), 8.47 (s, 1H), 7.86 (d, J = 7.9 Hz, 1H), |
| | | | 7.77 (br s, 1H), 7.73-7.61 (m, 3H), 7.60-7.56 (m, 1H), 7.55- |
| | | | 7.51 (m, 1H), 7.30 (br s, 1H), 7.26-7.16 (m, 1H), 7.10 (br d, J = |
| | | | 7.9 Hz, 1H), 6.51 (d, J = 9.1 Hz, 1H), 6.35 (t, J = 6.6 Hz, 1H), |
| | | | 6.17 (br s, 1H), 5.43-5.30 (m, 1H), 4.35-4.15 (m, 2H), 4.00 (br |
| | | | d, J = 11.3 Hz, 2H), 3.27-3.06 (m, 4H), 3.03-2.79 (m, 5H), 2.76- |
| | | | 2.57 (m, 5H), 2.46-2.30 (m, 1H), 2.29-2.08 (m, 2H), 2.08- |
| | | | 1.67 (m, 9H), 1.59-1.13 (m, 5H) |
| 374 | 793 | 794 | δ (DMSO-d6) 10.78 (s, 1H), 8.46 (br d, J = 3.3 Hz, 1H), 8.06 (br |
| | | | d, J = 7.5 Hz, 1H), 7.97 (br s, 1H), 7.74-7.66 (m, 2H), 7.62- |
| | | | 7.51 (m, 2H), 7.28 (br d, J = 7.5 Hz, 1H), 6.85 (br t, J = 9.3 Hz, |
| | | | 1H), 6.56-6.48 (m, 2H), 6.43 (br d, J = 8.4 Hz, 1H), 6.35 (t, J = |
| | | | 6.4 Hz, 1H), 5.87 (br d, J = 6.8 Hz, 1H), 4.45-4.33 (m, 1H), 4.32- |
| | | | 4.21 (m, 1H), 3.95 (br d, J = 12.6 Hz, 1H), 3.71-3.58 (m, 1H), |
| | | | 3.14-2.89 (m, 8H), 2.73 (ddd, J = 5.0, 12.1, 17.4 Hz, 4H), 2.13- |
| | | | 1.91 (m, 5H), 1.82-1.62 (m, 5H), 1.53-1.25 (m, 5H), 1.17- |
| | | | 0.85 (m, 3H) |
| 375 | 807 | 808 | δ (DMSO-d6) 10.81 (s, 1H), 8.48 (d, J = 3.5 Hz, 1H), 8.15 (s, |
| | | | 1H), 8.08 (br d, J = 7.0 Hz, 1H), 7.99 (br s, 1H), 7.76-7.68 (m, |
| | | | 3H), 7.62 (br d, J = 8.1 Hz, 1H), 7.59-7.52 (m, 1H), 7.26 (br d, |
| | | | J = 7.6 Hz, 1H), 6.84 (t, J = 9.3 Hz, 1H), 6.56-6.51 (m, 1H), 6.45- |
| | | | 6.40 (m, 1H), 6.37 (t, J = 6.7 Hz, 1H), 5.82 (br d, J = 7.6 Hz, |
| | | | 1H), 4.31-4.22 (m, 1H), 3.72-3.56 (m, 3H), 3.13 (br d, J = 10.9 |
| | | | Hz, 3H), 3.06-2.97 (m, 1H), 2.81-2.67 (m, 2H), 2.66-2.55 (m, |
| | | | 4H), 2.14-1.95 (m, 4H), 1.92-1.62 (m, 9H), 1.56-1.40 (m, 4H), |
| | | | 1.35-1.21 (m, 4H) |
| 376 | 793 | 794 | δ (DMSO-d6) 11.86 (br s, 1H), 10.77 (s, 1H), 8.47-8.27 (m, 2H), |
| | | | 7.93-7.83 (m, 2H), 7.70 (dd, J = 2.0, 6.9 Hz, 1H), 7.55 (s, 1H), |
| | | | 7.46-7.40 (m, 1H), 7.24-7.18 (m, 1H), 6.87-6.77 (m, 1H), 6.54- |
| | | | 6.47 (m, 1H), 6.45-6.38 (m, 1H), 6.32 (s, 1H), 5.80 (br d, J = |
| | | | 7.6 Hz, 1H), 4.42-4.33 (m, 1H), 4.30-4.20 (m, 1H), 3.98-3.88 |
| | | | (m, 1H), 3.72-3.59 (m, 1H), 3.05-2.94 (m, 1H), 2.85 (br s, 4H), |
| | | | 2.77 (br s, 1H), 2.58 (br d, J = 4.1 Hz, 1H), 2.56-2.52 (m, 2H), |
| | | | 2.48-2.45 (m, 2H), 2.22-2.13 (m, 2H), 2.11-2.05 (m, 1H), 2.04- |
| | | | 1.94 (m, 2H), 1.92-1.76 (m, 3H), 1.70 (br d, J = 12.4 Hz, 4H), |
| | | | 1.54-1.43 (m, 2H), 1.43-1.27 (m, 3H), 1.01 (br d, J = 12.3 Hz, |
| | | | 1H), 0.89 (br d, J = 10.8 Hz, 1H) |
| 377 | 821 | 822 | δ 10.77 (s, 1H), 8.45 (d, J = 3.5 Hz, 1H), 8.21 (s, 1H), 8.03 (br d, |
| | | | J = 8.4 Hz, 1H), 7.95 (br s, 1H), 7.76-7.65 (m, 2H), 7.62-7.48 |
| | | | (m, 2H), 7.21 (br d, J = 7.5 Hz, 1H), 7.05 (br d, J = 7.4 Hz, 1H), |
| | | | 6.81 (br t, J = 9.2 Hz, 1H), 6.57-6.45 (m, 2H), 6.44-6.30 (m, |
| | | | 2H), 5.78 (br d, J = 7.6 Hz, 1H), 4.72 (br t, J = 6.9 Hz, 1H), 4.38- |
| | | | 4.12 (m, 1H), 3.67-3.56 (m, 1H), 3.15-2.99 (m, 6H), 2.79- |
| | | | 2.64 (m, 2H), 2.35-2.28 (m, 1H), 2.16-1.88 (m, 7H), 1.87-1.56 |
| | | | (m, 6H), 1.39-1.14 (m, 7H) |
| 378 | 832 | 833 | δ 11.19 (s, 1H), 8.48 (d, J = 3.5 Hz, 1H), 8.16 (s, 1H), 8.05 (br d, |
| | | | J = 7.2 Hz, 1H), 7.98 (s, 1H), 7.76-7.64 (m, 2H), 7.62-7.58 (m, |
| | | | 1H), 7.54 (ddd, J = 2.0, 6.8, 9.1 Hz, 1H), 7.40 (d, J = 7.6 Hz, 1H), |
| | | | 7.10-7.02 (m, 2H), 6.76 (dd, J = 2.0, 8.8 Hz, 1H), 6.51 (d, J = |
| | | | 9.2 Hz, 1H), 6.35 (dt, J = 1.2, 6.7 Hz, 1H), 5.29 (dd, J = 5.2, 13.0 |
| | | | Hz, 1H), 4.23 (br d, J = 13.1 Hz, 1H), 4.07-3.88 (m, 2H), 3.61- |
| | | | 3.53 (m, 2H), 3.20-3.05 (m, 2H), 3.01 (br d, J = 12.7 Hz, 1H), |
| | | | 2.96-2.69 (m, 3H), 2.69-2.53 (m, 5H), 2.47-2.28 (m, 6H), 2.19- |
| | | | 2.07 (m, 3H), 1.99-1.83 (m, 2H), 1.75 (br d, J = 11.1 Hz, 2H), |
| | | | 1.69-1.56 (m, 1H), 1.48 (br dd, J = 1.7, 9.8 Hz, 1H), 1.39-1.26 |
| | | | (m, 1H), 1.26-1.09 (m, 2H) |
| 379 | 831 | 832 | δ 12.02-11.71 (m, 1H), 11.19 (s, 1H), 8.44 (d, J = 3.6 Hz, 1H), |
| | | | 8.35 (br s, 1H), 8.15 (s, 1H), 7.92-7.81 (m, 2H), 7.71 (dd, J = |
| | | | 1.9, 6.9 Hz, 1H), 7.55 (t, J = 7.8 Hz, 1H), 7.42 (dd, J = 1.8, 6.4 |
| | | | Hz, 1H), 7.37-7.27 (m, 2H), 7.20-7.13 (m, 1H), 7.11-7.05 (m, |
| | | | 1H), 6.32 (t, J = 6.6 Hz, 1H), 5.34 (dd, J = 5.2, 12.8 Hz, 1H), 4.26 |
| | | | (br d, J = 12.9 Hz, 1H), 4.09-4.02 (m, 1H), 4.01-3.92 (m, 1H), |
| | | | 3.17 (br s, 2H), 3.03 (br d, J = 13.5 Hz, 1H), 2.94 (br d, J = 10.4 |
| | | | Hz, 3H), 2.90-2.74 (m, 4H), 2.72-2.61 (m, 2H), 2.14-2.13 (m, |
| | | | 3H), 2.07-1.89 (m, 6H), 1.77-1.59 (m, 6H), 1.56-1.43 (m, 2H), |
| | | | 1.39-1.24 (m, 1H), 1.23-1.04 (m, 2H) |
| 380 | 847 | 848 | δ 11.16 (br s, 1H), 8.48 (d, J = 3.6 Hz, 1H), 8.05 (br d, J = 8.0 Hz, |
| | | | 1H), 7.97 (s, 1H), 7.75-7.65 (m, 2H), 7.61-7.50 (m, 2H), 7.37 |
| | | | (d, J = 7.6 Hz, 1H), 7.09-6.99 (m, 2H), 6.76 (dd, J = 2.1, 8.8 Hz, |
| | | | 1H), 6.51 (d, J = 9.0 Hz, 1H), 6.35 (dt, J = 1.1, 6.8 Hz, 1H), 5.28 |
| | | | (dd, J = 5.4, 12.9 Hz, 1H), 4.82 (s, 1H), 4.38-4.24 (m, 1H), 4.06- |
| | | | 3.92 (m, 2H), 3.57 (br d, J = 12.1 Hz, 2H), 3.22-3.11 (m, 2H), |
| | | | 2.93-2.75 (m, 2H), 2.69-2.56 (m, 5H), 2.35-2.21 (m, 3H), 2.18- |
| | | | 2.05 (m, 3H), 1.95-1.86 (m, 2H), 1.75 (br d, J = 12.1 Hz, 2H), |
| | | | 1.63-1.50 (m, 6H), 1.37-1.27 (m, 1H), 1.25-1.01 (m, 3H) |
| 381 | 793 | 794 | δ (DMSO-d6) 10.78 (s, 1H), 8.46 (br d, J = 3.2 Hz, 1H), 8.06 (br |
| | | | d, J = 7.1 Hz, 1H), 7.96 (br s, 1H), 7.74-7.66 (m, 2H), 7.61- |
| | | | 7.51 (m, 3H), 7.22 (br d, J = 7.8 Hz, 1H), 6.80 (br t, J = 9.4 Hz, |
| | | | 1H), 6.54-6.47 (m, 2H), 6.41 (br d, J = 8.4 Hz, 1H), 6.35 (t, J = |
| | | | 6.7 Hz, 1H), 5.79 (br d, J = 7.6 Hz, 1H), 4.25 (br s, 1H), 3.69- |
| | | | 3.59 (m, 1H), 3.46-3.39 (m, 1H), 2.82 (br s, 4H), 2.60 (br s, 4H), |
| | | | 2.21 (br dd, J = 8.6, 6.2 Hz, 1H), 2.12-2.01 (m, 2H), 2.01-1.94 |
| | | | (m, 2H), 1.80 (br d, J = 9.8 Hz, 5H), 1.71 (br d, J = 11.9 Hz, 2H), |
| | | | 1.43 (br d, J = 11.2 Hz, 2H), 1.33-1.21 (m, 5H), 1.15-1.15 (m, |
| | | | 1H), 1.15 (br d, J = 11.1 Hz, 2H) |
| 382 | 793 | 794 | δ (DMSO-d6) 11.95-11.83 (m, 1H), 10.79 (s, 1H), 8.42 (d, J = |
| | | | 3.5 Hz, 1H), 8.32 (br s, 1H), 8.14 (s, 1H), 7.90-7.84 (m, 2H), |
| | | | 7.70 (dd, J = 2.0, 6.8 Hz, 1H), 7.58 (br dd, J = 12.6, 7.9 Hz, 2H), |
| | | | 7.43 (br d, J = 6.5 Hz, 1H), 7.16 (br d, J = 7.7 Hz, 1H), 6.81 (t, |
| | | | J = 9.4 Hz, 1H), 6.50 (dd, J = 15.0, 1.9 Hz, 1H), 6.41 (br d, J = 8.8 |
| | | | Hz, 1H), 6.32 (t, J = 6.7 Hz, 1H), 5.81 (br d, J = 7.3 Hz, 1H), 4.25 |
| | | | (br s, 1H), 3.70-3.60 (m, 2H), 2.85 (br s, 4H), 2.72 (br d, J = 5.7 |
| | | | Hz, 1H), 2.67 (br s, 4H), 2.58 (br d, J = 4.0 Hz, 1H), 2.33 (br s, |
| | | | 1H), 2.11-2.03 (m, 2H), 2.02-1.97 (m, 2H), 1.85-1.80 (m, 4H), |
| | | | 1.75-1.69 (m, 2H), 1.44 (q, J = 12.2 Hz, 2H), 1.28 (br t, J = 11.3 |
| | | | Hz, 4H), 1.16 (br d, J = 10.9 Hz, 2H) |
| 383 | 814 | 815 | δ (DMSO-d6) 10.85 (s, 1H), 8.46 (br d, J = 3.4 Hz, 1H), 8.20 (s, |
| | | | 1H), 8.07 (br d, J = 7.1 Hz, 1H), 8.01-7.90 (m, 1H), 7.74-7.68 |
| | | | (m, 2H), 7.63-7.50 (m, 4H), 7.22 (br d, J = 7.8 Hz, 1H), 6.92 (br |
| | | | d, J = 8.5 Hz, 1H), 6.83 (s, 1H), 6.52 (d, J = 9.1 Hz, 1H), 6.36 (t, |
| | | | J = 6.8 Hz, 1H), 4.26 (br dd, J = 9.1, 5.1 Hz, 2H), 3.89 (s, 3H), |
| | | | 3.20 (br s, 4H), 2.67 (br s, 4H), 2.61 (br s, 1H), 2.34-2.25 (m, |
| | | | 4H), 2.18-2.15 (m, 1H), 2.03-1.96 (m, 3H), 1.87-1.81 (m, 4H), |
| | | | 1.75-1.70 (m, 2H), 1.48-1.41 (m, 2H), 1.33-1.24 (m, 4H), 1.21- |
| | | | 1.14 (m, 2H) |
| 384 | 792 | 793 | δ (DMSO-d6) 11.96-11.81 (m, 1H), 10.79 (s, 1H), 8.43 (d, J = |
| | | | 3.5 Hz, 1H), 8.34 (br s, 1H), 7.89-7.85 (m, 2H), 7.72-7.69 (m, |
| | | | 1H), 7.56 (t, J = 7.9 Hz, 1H), 7.43 (dd, J = 1.5, 6.2 Hz, 1H), 7.22 |
| | | | (br d, J = 7.5 Hz, 1H), 7.01-6.95 (m, 1H), 6.47-6.44 (m, 1H), |
| | | | 6.33 (t, J = 6.7 Hz, 2H), 6.00 (br d, J = 7.6 Hz, 1H), 4.39 (br d, |
| | | | J = 11.9 Hz, 1H), 4.34-4.27 (m, 1H), 3.96-3.91 (m, 1H), 3.71- |
| | | | 3.63 (m, 2H), 3.03-2.97 (m, 1H), 2.91 (br d, J = 8.6 Hz, 2H), |
| | | | 2.79-2.70 (m, 1H), 2.14 (br d, J = 6.2 Hz, 4H), 2.03-1.99 (m, |
| | | | 2H), 1.94 (br d, J = 3.1 Hz, 2H), 1.79-1.76 (m, 2H), 1.71 (br d, |
| | | | J = 12.8 Hz, 4H), 1.63 (br s, 6H), 1.48-1.42 (m, 2H), 1.35 (br d, |
| | | | J = 4.2 Hz, 2H), 1.00 (br dd, J = 2.5, 11.2 Hz, 1H), 0.88 (br dd, |
| | | | J = 3.9, 7.0 Hz, 1H) |
| 385 | 813 | 814 | δ (DMSO-d6) 8.47 (d, J = 3.5 Hz, 1H), 8.07 (br d, J = 7.8 Hz, |
| | | | 1H), 7.98 (s, 1H), 7.75-7.68 (m, 2H), 7.60 (d, J = 8.1 Hz, 2H), |
| | | | 7.55 (ddd, J = 9.1, 6.8, 2.0 Hz, 1H), 7.45 (s, 1H), 7.31-7.25 (m, |
| | | | 1H), 7.04 (d, J = 8.6 Hz, 1H), 6.52 (d, J = 9.2 Hz, 1H), 6.36 (dt, |
| | | | J = 6.7, 1.1 Hz, 1H), 4.40 (br d, J = 12.3 Hz, 1H), 4.33 (dd, J = 9.8, |
| | | | 5.1 Hz, 1H), 3.97 (s, 3H), 3.93 (br s, 1H), 3.71-3.62 (m, 1H), |
| | | | 3.04-2.93 (m, 2H), 2.74-2.57 (m, 3H), 2.56 (br s, 1H), 2.53- |
| | | | 2.52 (m, 2H), 2.35 (tdd, J = 13.8, 9.2, 4.8 Hz, 1H), 2.19-2.14 (m, |
| | | | 2H), 2.05-1.94 (m, 4H), 1.79 (br d, J = 2.7 Hz, 5H), 1.71 (br d, |
| | | | J = 11.5 Hz, 3H), 1.54-1.41 (m, 2H), 1.39-1.28 (m, 2H), 1.27- |
| | | | 1.19 (m, 1H), 1.19-1.10 (m, 1H), 1.08-0.99 (m, 1H), 0.96-0.84 |
| | | | (m, 1H) |
| 386 | 831 | 832 | δ 11.23-11.08 (m, 1H), 8.55-8.38 (m, 1H), 8.09-8.01 (m, 1H), |
| | | | 7.97 (br s, 1H), 7.71 (br d, J = 5.6 Hz, 2H), 7.60 (br s, 1H), 7.57- |
| | | | 7.50 (m, 1H), 7.33-7.25 (m, 1H), 7.05 (br s, 2H), 6.83-6.71 |
| | | | (m, 1H), 6.57-6.46 (m, 1H), 6.41-6.28 (m, 1H), 5.28 (br dd, |
| | | | J = 2.0, 5.8 Hz, 1H), 3.87-3.74 (m, 4H), 3.59-3.54 (m, 4H), 2.92- |
| | | | 2.80 (m, 7H), 2.01-1.84 (m, 4H), 1.73 (br d, J = 12.4 Hz, 4H), |
| | | | 1.60 (br dd, J = 5.7, 4.4 Hz, 2H), 1.48-1.34 (m, 5H), 1.31-1.19 |
| | | | (m, 5H) |
| 387 | 793 | 794 | δ 10.78 (s, 1H), 8.77 (dd, J = 1.3, 4.3 Hz, 1H) 8.06 (br d, J = 7.6 |
| | | | Hz, 1H), 7.97 (br s, 1H), 7.74-7.66 (m, 2H), 7.63-7.49 (m, 2H), |
| | | | 7.31 (br d, J = 7.5 Hz, 1H), 6.87-6.79 (m, 1H), 6.59-6.27 (m, |
| | | | 4H), 5.80 (br d, J = 8.0 Hz, 1H), 4.36-4.16 (m, 1H), 3.74-3.48 |
| | | | (m, 4H), 3.19-3.02 (m, 3H), 2.78-2.64 (m, 4H), 2.58 (br d, J = |
| | | | 3.8 Hz, 4H), 2.16-1.94 (m, 4H), 1.90-1.65 (m, 7H), 1.54-1.22 |
| | | | (m, 7H) |
| 388 | 814 | 815 | δ (DMSO-d6) 10.86 (s, 1H), 8.47 (d, J = 3.5 Hz, 1H), 8.05 (br d, |
| | | | J = 7.4 Hz, 1H), 7.98 (br s, 1H), 7.75-7.71 (m, 1H), 7.71-7.68 |
| | | | (m, 1H), 7.60 (br d, J = 8.0 Hz, 2H), 7.55 (ddd, J = 9.1, 6.7, 2.0 |
| | | | Hz, 1H), 7.50 (d, J = 9.0 Hz, 1H), 7.23 (br d, J = 7.8 Hz, 1H), |
| | | | 6.94-6.89 (m, 1H), 6.83 (s, 1H), 6.53 (d, J = 9.1 Hz, 1H), 6.36 |
| | | | (dt, J = 6.7, 1.2 Hz, 1H), 4.26 (dd, J = 9.2, 5.2 Hz, 1H), 3.90 (s, |
| | | | 3H), 3.69-3.60 (m, 1H), 3.54-3.44 (m, 1H), 3.20 (br s, 4H), |
| | | | 2.68 (br s, 4H), 2.62 (br d, J = 5.5 Hz, 1H), 2.55 (s, 1H), 2.34- |
| | | | 2.26 (m, 2H), 2.16 (br dd, J = 12.8, 5.8 Hz, 1H), 1.97 (br d, J = |
| | | | 11.4 Hz, 3H), 1.90 (br d, J = 11.0 Hz, 2H), 1.78 (br s, 4H), 1.39- |
| | | | 1.21 (m, 8H) |
| 389 | 814 | 815 | δ (DMSO-d6) 10.87 (s, 1H), 8.43 (d, J = 3.6 Hz, 1H), 8.32 (s, |
| | | | 1H), 8.17 (s, 1H), 7.91-7.86 (m, 2H), 7.71 (dd, J = 6.9, 2.0 Hz, |
| | | | 1H), 7.61 (br d, J = 8.0 Hz, 1H), 7.56 (t, J = 7.8 Hz, 1H), 7.50 (d, |
| | | | J = 9.0 Hz, 1H), 7.44 (dd, J = 6.4, 2.0 Hz, 1H), 7.17 (br d, J = 7.8 |
| | | | Hz, 1H), 6.95-6.90 (m, 1H), 6.83 (s, 1H), 6.34 (t, J = 6.6 Hz, |
| | | | 1H), 4.26 (dd, J = 9.1, 5.1 Hz, 1H), 3.90 (s, 3H), 3.67-3.64 (m, |
| | | | 1H), 3.50 (br d, J = 7.5 Hz, 2H), 2.68 (br s, 4H), 2.62 (br d, J = |
| | | | 5.5 Hz, 2H), 2.34-2.28 (m, 2H), 2.20-2.13 (m, 1H), 1.99 (br d, |
| | | | J = 7.3 Hz, 3H), 1.90 (br d, J = 10.3 Hz, 2H), 1.77 (br d, J = 9.8 |
| | | | Hz, 5H), 1.45-1.34 (m, 4H), 1.31 (br d, J = 10.5 Hz, 2H), 1.28- |
| | | | 1.20 (m, 4H) |
| 390 | 828 | 829 | δ (DMSO-d6) 8.47 (d, J = 3.3 Hz, 1H), 8.07 (br d, J = 7.7 Hz, |
| | | | 1H), 7.98 (br s, 1H), 7.76-7.67 (m, 2H), 7.63-7.52 (m, 2H), |
| | | | 7.30 (br d, J = 7.7 Hz, 1H), 7.08 (br t, J = 8.5 Hz, 1H), 6.58-6.42 |
| | | | (m, 3H), 6.36 (t, J = 6.7 Hz, 1H), 6.18 (br d, J = 7.4 Hz, 1H), 4.44- |
| | | | 4.31 (m, 2H), 4.01-3.89 (m, 1H), 3.71-3.58 (m, 1H), 3.25- |
| | | | 3.08 (m, 2H), 3.07-2.88 (m, 2H), 2.82-2.66 (m, 2H), 2.40-2.22 |
| | | | (m, 4H), 2.21-2.05 (m, 3H), 1.99 (br d, J = 10.4 Hz, 2H), 1.86- |
| | | | 1.77 (m, 2H), 1.76-1.62 (m, 5H), 1.55-1.25 (m, 5H), 1.06-0.85 |
| | | | (m, 2H) |
| 391 | 813 | 814 | δ (DMSO-d6) 1.87 (br s, 1H), 10.89 (s, 1H), 8.49-8.29 (m, 2H), |
| | | | 7.97-7.84 (m, 2H), 7.71 (dd, J = 6.9, 1.8 Hz, 1H), 7.65-7.53 |
| | | | (m, 2H), 7.49-7.41 (m, 2H), 7.23 (br d, J = 7.8 Hz, 1H), 7.04 (d, |
| | | | J = 8.5 Hz, 1H), 6.33 (t, J = 6.7 Hz, 1H), 4.45-4.37 (m, 1H), 4.33 |
| | | | (dd, J = 9.7, 5.1 Hz, 1H), 3.97 (s, 4H), 3.72-3.61 (m, 1H), 3.08- |
| | | | 2.92 (m, 3H), 2.73-2.56 (m, 5H), 2.40-2.31 (m, 1H), 2.23- |
| | | | 2.10 (m, 3H), 2.01 (br s, 4H), 1.85-1.67 (m, 9H), 1.54-1.43 (m, |
| | | | 2H), 1.42-1.28 (m, 2H), 1.10-0.84 (m, 2H) |
| 392 | 809 | 810 | δ (DMSO-d6) 10.92-10.67 (m, 1H), 8.46 (d, J = 3.5 Hz, 1H), |
| | | | 8.06 (br d, J = 7.1 Hz, 1H), 7.97 (br s, 1H), 7.74-7.68 (m, 2H), |
| | | | 7.59 (dd, J = 0.9, 7.9 Hz, 1H), 7.54 (ddd, J = 2.0, 6.7, 9.1 Hz, 1H), |
| | | | 7.30 (br d, J = 7.5 Hz, 1H), 6.81 (t, J = 9.4 Hz, 1H), 6.54-6.46 |
| | | | (m, 2H), 6.43-6.38 (m, 1H), 6.37-6.32 (m, 1H), 5.79 (d, J = 7.5 |
| | | | Hz, 1H), 4.31-4.19 (m, 2H), 4.06-3.97 (m, 1H), 3.64 (br s, 2H), |
| | | | 3.01-2.90 (m, 1H), 2.82 (br s, 4H), 2.73-2.62 (m, 5H), 2.57 (br |
| | | | d, J = 4.0 Hz, 2H), 2.30 (s, 2H), 2.13-2.04 (m, 1H), 2.03-1.96 |
| | | | (m, 2H), 1.88-1.78 (m, 1H), 1.68 (br s, 2H), 1.44 (br s, 6H), 1.36 |
| | | | (br d, J = 11.7 Hz, 3H) |
| 393 | 797 | 798 | δ (DMSO-d6) 10.78 (s, 1H), 8.43 (d, J = 3.6 Hz, 1H), 7.90-7.81 |
| | | | (m, 2H), 7.59-7.53 (m, 1H), 7.45 (br d, J = 8.0 Hz, 1H), 7.25 (br |
| | | | d, J = 7.6 Hz, 1H), 6.84 (t, J = 9.3 Hz, 1H), 6.52 (dd, J = 2.2, 15.2 |
| | | | Hz, 1H), 6.46-6.39 (m, 1H), 5.82 (br d, J = 7.3 Hz, 1H), 4.39 (br |
| | | | d, J = 13.1 Hz, 1H), 4.26 (ddd, J = 4.9, 7.2, 11.6 Hz, 1H), 3.95 (br |
| | | | d, J = 11.1 Hz, 1H), 3.65 (br t, J = 5.3 Hz, 3H), 3.00 (br dd, J = |
| | | | 6.4, 12.9 Hz, 2H), 2.89 (br s, 4H), 2.79-2.66 (m, 2H), 2.62-2.54 |
| | | | (m, 4H), 2.42 (br t, J = 6.3 Hz, 3H), 2.09 (td, J = 4.2, 8.1 Hz, 2H), |
| | | | 2.00 (br d, J = 9.8 Hz, 2H), 1.93-1.79 (m, 7H), 1.71 (br d, J = |
| | | | 10.0 Hz, 4H), 1.53-1.41 (m, 2H), 1.40-1.28 (m, 2H), 1.09-0.98 |
| | | | (m, 1H), 0.96-0.85 (m, 1H) |
| 394 | 796 | 797 | δ (DMSO-d6) 10.84-10.71 (m, 1H), 8.43 (br s, 1H), 7.89-7.84 |
| | | | (m, 2H), 7.56 (t, J = 7.9 Hz, 1H), 7.46 (br d, J = 7.7 Hz, 1H), 7.23 |
| | | | (br d, J = 7.7 Hz, 1H), 7.02-6.97 (m, 1H), 6.47-6.42 (m, 2H), |
| | | | 6.01-5.97 (m, 1H), 4.42-4.35 (m, 1H), 4.33-4.27 (m, 1H), 3.97- |
| | | | 3.91 (m, 1H), 3.65 (br t, J = 5.4 Hz, 3H), 3.04-2.97 (m, 1H), |
| | | | 2.92 (br d, J = 10.5 Hz, 2H), 2.81-2.65 (m, 2H), 2.59 (br d, J = |
| | | | 3.8 Hz, 2H), 2.56 (br d, J = 3.7 Hz, 2H), 2.44-2.41 (m, 2H), 2.15 |
| | | | (br d, J = 6.2 Hz, 2H), 2.13-2.05 (m, 2H), 2.04-1.95 (m, 4H), |
| | | | 1.88 (br d, J = 4.4 Hz, 3H), 1.81-1.76 (m, 2H), 1.71 (br d, J = |
| | | | 12.5 Hz, 3H), 1.63 (br s, 4H), 1.51-1.43 (m, 2H), 1.40-1.32 (m, |
| | | | 2H), 1.06-0.97 (m, 1H), 0.93-0.84 (m, 1H) |
| 395 | 807 | 808 | δ 8.55 (s, 1H), 8.25 (d, J = 3.5 Hz, 1H), 8.17-8.05 (m, 3H), 7.70- |
| | | | 7.53 (m, 2H), 7.50-7.37 (m, 2H), 6.86 (t, J = 9.3 Hz, 1H), 6.71 |
| | | | (d, J = 9.3 Hz, 1H), 6.50-6.36 (m, 2H), 6.34-6.25 (m, 1H), 5.58 |
| | | | (br d, J = 7.0 Hz, 1H), 5.03 (d, J = 8.0 Hz, 1H), 4.64 (br s, 1H), |
| | | | 4.02 (dd, J = 12.4, 4.6 Hz, 1H), 3.81 (br d, J = 1.4 Hz, 2H), 3.39 |
| | | | (br d, J = 10.3 Hz, 4H), 3.16-3.07 (m, 1H), 2.93-2.85 (m, 1H), |
| | | | 2.81-2.68 (m, 4H), 2.58-2.46 (m, 1H), 2.25-2.04 (m, 8H), 1.99- |
| | | | 1.85 (m, 5H), 1.83-1.77 (m, 1H), 1.35 (br t, J = 10.1 Hz, 4H) |
| 396 | 792 | 793 | δ 12.58 (s, 1H), 10.79 (s, 1H), 8.41 (d, J = 3.5 Hz, 1H), 8.32 (br |
| | | | s, 1H), 8.19 (s, 0.3H), 7.94-7.83 (m, 2H), 7.70 (br d, J = 5.8 Hz, |
| | | | 2H), 7.60-7.50 (m, 1H), 7.44 (br s, 1H), 7.15 (br d, J = 7.6 Hz, |
| | | | 1H), 7.08-6.90 (m, 1H), 6.53-6.40 (m, 2H), 6.39-6.26 (m, 1H), |
| | | | 6.16-5.95 (m, 1H), 4.38-4.23 (m, 2H), 3.65 (br s, 1H), 3.52- |
| | | | 3.44 (m, 1H), 2.79-2.65 (m, 4H), 2.15-2.04 (m, 2H), 1.98 (br |
| | | | s, 4H), 1.86 (br dd, J = 4.4, 12.4 Hz, 2H), 1.78 (br d, J = 7.0 Hz, |
| | | | 5H), 1.53-1.41 (m, 4H), 1.37-1.24 (m, 6H), 0.90-0.75 (m, 3H) |
| 397 | 817 | 818 | δ 11.18 (s, 1H), 8.46 (br s, 1H), 8.07 (br d, J = 7.1 Hz, 1H), 7.98 |
| | | | (br s, 1H), 7.81-7.65 (m, 2H), 7.64-7.47 (m, 2H), 7.28 (br d, |
| | | | J = 7.1 Hz, 1H), 7.17-6.96 (m, 2H), 6.79 (br d, J = 8.6 Hz, 1H), |
| | | | 6.52 (br d, J = 9.3 Hz, 1H), 6.36 (br t, J = 6.5 Hz, 1H), 5.76 (d, |
| | | | J = 1.9 Hz, 1H), 5.30 (br dd, J = 3.9, 12.7 Hz, 1H), 4.39 (br d, J = |
| | | | 11.9 Hz, 1H), 3.94 (br d, J = 12.3 Hz, 1H), 3.64 (br s, 2H), 3.11 |
| | | | (br s, 4H), 2.99-2.83 (m, 2H), 2.67 (br d, J = 5.1 Hz, 2H), 2.55 |
| | | | (br s, 3H), 2.25 (br s, 2H), 2.14 (br d, J = 6.0 Hz, 1H), 1.99 (br d, |
| | | | J = 10.1 Hz, 2H), 1.79 (br d, J = 11.5 Hz, 2H), 1.70 (br d, J = 10.9 |
| | | | Hz, 3H), 1.61-1.19 (m, 5H), 1.10-0.85 (m, 2H) |
| 398 | 817 | 818 | δ 12.09-11.76 (m, 1H), 11.20 (s, 1H), 8.43 (d, J = 3.5 Hz, 1H), |
| | | | 8.33 (br d, J = 2.2 Hz, 1H), 7.98-7.83 (m, 2H), 7.71 (dd, J = 2.0, |
| | | | 6.8 Hz, 1H), 7.56 (t, J = 7.6 Hz, 1H), 7.47-7.38 (m, 1H), 7.25 |
| | | | (br d, J = 7.9 Hz, 1H), 7.19-7.02 (m, 2H), 6.90-6.70 (m, 1H), |
| | | | 6.33 (t, J = 6.7 Hz, 1H), 5.41-5.19 (m, 1H), 4.40 (br dd, J = 3.8, |
| | | | 10.8 Hz, 1H), 4.06-3.86 (m, 1H), 3.72-3.58 (m, 1H), 3.08 (br |
| | | | s, 6H), 2.92-2.80 (m, 1H), 2.76-2.54 (m, 4H), 2.38-2.31 (m, |
| | | | 1H), 2.28-2.07 (m, 3H), 2.05-1.95 (m, 2H), 1.93-1.60 (m, 6H), |
| | | | 1.40 (br s, 5H), 1.16-0.85 (m, 2H) |
| 399 | 814 | 815 | δ 10.87 (s, 1H), 8.46 (d, J = 3.3 Hz, 1H), 8.13 (s, 0.14H), 8.06 (br |
| | | | d, J = 7.8 Hz, 1H), 7.97 (br s, 1H), 7.76-7.66 (m, 2H), 7.63- |
| | | | 7.49 (m, 3H), 7.31 (br d, J = 7.8 Hz, 1H), 7.01-6.79 (m, 2H), |
| | | | 6.51 (d, J = 9.3 Hz, 1H), 6.35 (t, J = 6.5 Hz, 1H), 4.40 (br d, J = |
| | | | 11.9 Hz, 1H), 4.27 (br dd, J = 5.1, 9.1 Hz, 1H), 4.04-3.81 (m, |
| | | | 5H), 3.72-3.56 (m, 2H), 3.27-3.10 (m, 3H), 3.08-2.92 (m, 2H), |
| | | | 2.73-2.57 (m, 4H), 2.42-2.07 (m, 4H), 2.06-1.92 (m, 3H), 1.88- |
| | | | 1.60 (m, 5H), 1.53-1.23 (m, 5H), 1.14-0.90 (m, 2H) |
| 400 | 792 | 793 | δ 10.77 (s, 1H), 8.46 (d, J = 3.5 Hz, 1H), 8.06 (br d, J = 8.0 Hz, |
| | | | 1H), 7.97 (s, 1H), 7.75-7.65 (m, 2H), 7.62-7.49 (m, 2H), 7.27 |
| | | | (d, J = 7.8 Hz, 1H), 6.87-6.75 (m, 1H), 6.54-6.46 (m, 2H), 6.41 |
| | | | (br d, J = 7.4 Hz, 1H), 6.35 (dt, J = 1.2, 6.7 Hz, 1H), 5.81-5.73 |
| | | | (m, 1H), 4.39 (br d, J = 12.1 Hz, 1H), 4.25 (br d, J = 5.0 Hz, 1H), |
| | | | 3.98-3.86 (m, 1H), 3.64 (br dd, J = 2.8, 4.6 Hz, 1H), 3.10 (br dd, |
| | | | J = 7.3, 1.8 Hz, 2H), 3.04-2.92 (m, 1H), 2.79-2.67 (m, 1H), |
| | | | 2.58 (br d, J = 4.3 Hz, 1H), 2.56-2.52 (m, 3H), 2.06 (br d, J = |
| | | | 4.4 Hz, 1H), 1.99 (br d, J = 10.1 Hz, 2H), 1.92-1.81 (m, 1H), |
| | | | 1.76-1.65 (m, 6H), 1.65-1.54 (m, 2H), 1.43 (br d, J = 11.3 Hz, |
| | | | 3H), 1.37-1.20 (m, 4H), 1.17 (br d, J = 6.4 Hz, 2H), 1.06-0.94 |
| | | | (m, 1H), 0.88 (br dd, J = 2.1, 9.3 Hz, 1H) |
| 401 | 792 | 793 | δ (DMSO-d6) 10.80 (s, 1H), 8.56-8.41 (m, 1H), 8.07 (br d, J = |
| | | | 7.6 Hz, 1H), 7.98 (br s, 1H), 7.76-7.68 (m, 2H), 7.61 (br d, J = |
| | | | 8.4 Hz, 1H), 7.59-7.52 (m, 2H), 7.43-7.32 (m, 1H), 7.26-7.15 |
| | | | (m, 1H), 7.05-6.93 (m, 1H), 6.53 (d, J = 9.4 Hz, 1H), 6.48-6.41 |
| | | | (m, 1H), 6.40-6.31 (m, 1H), 6.00 (br d, J = 7.8 Hz, 1H), 4.40- |
| | | | 4.23 (m, 1H), 3.73-3.57 (m, 1H), 3.49-3.38 (m, 1H), 2.96-2.86 |
| | | | (m, 2H), 2.80-2.71 (m, 1H), 2.64-2.57 (m, 1H), 2.33-2.20 (m, |
| | | | 3H), 2.15-1.93 (m, 4H), 1.57 (br s, 11H), 1.53-1.09 (m, 9H) |
| 402 | 792 | 793 | δ (DMSO-d6) 10.81 (s, 1H), 10.28-10.12 (m, 1H), 8.47-8.28 |
| | | | (m, 2H), 7.98-7.82 (m, 2H), 7.75-7.65 (m, 2H), 7.57 (t, J = 7.8 |
| | | | Hz, 1H), 7.50-7.42 (m, 1H), 6.97 (t, J = 8.7 Hz, 1H), 6.56-6.41 |
| | | | (m, 2H), 6.34 (t, J = 6.7 Hz, 1H), 4.41-4.25 (m, 1H), 3.52-3.37 |
| | | | (m, 3H), 3.23-3.06 (m, 3H), 3.04-2.93 (m, 1H), 2.82-2.67 (m, |
| | | | 1H), 2.59-2.54 (m, 1H), 2.22-1.98 (m, 9H), 1.93-1.83 (m, 5H), |
| | | | 1.75 (br d, J = 12.6 Hz, 2H), 1.67-1.53 (m, 2H), 1.53-1.40 (m, |
| | | | 2H), 1.36-1.21 (m, 4H) |
| 403 | 794 | 795 | δ (DMSO-d6) 12.03-11.65 (m, 1H), 10.97-10.61 (m, 1H), 8.48- |
| | | | 8.28 (m, 2H), 7.96-7.82 (m, 2H), 7.70 (dd, J = 6.9, 1.9 Hz, 1H), |
| | | | 7.55 (t, J = 7.8 Hz, 1H), 7.43 (dd, J = 6.4, 1.9 Hz, 1H), 7.21 (br |
| | | | d, J = 7.4 Hz, 1H), 6.83 (t, J = 9.4 Hz, 1H), 6.49 (dd, J = 14.9, 2.3 |
| | | | Hz, 1H), 6.40 (dd, J = 8.8, 2.0 Hz, 1H), 6.32 (t, J = 6.6 Hz, 1H), |
| | | | 5.78 (d, J = 7.5 Hz, 1H), 4.29-4.20 (m, 1H), 3.96-3.85 (m, 1H), |
| | | | 3.79-3.62 (m, 3H), 3.59-3.51 (m, 1H), 3.27-3.18 (m, 1H), 3.09- |
| | | | 2.99 (m, 3H), 2.74-2.63 (m, 3H), 2.60-2.54 (m, 2H), 2.12- |
| | | | 1.97 (m, 3H), 1.92-1.81 (m, 4H), 1.71 (br d, J = 12.6 Hz, 3H), |
| | | | 1.56 (br d, J = 9.0 Hz, 2H), 1.49-1.27 (m, 6H) |
| 404 | 814 | 815 | δ (DMSO-d6) 10.92-10.77 (m, 1H), 8.45-8.31 (m, 2H), 7.92- |
| | | | 7.85 (m, 2H), 7.72-7.69 (m, 1H), 7.60-7.53 (m, 2H), 7.49 (d, |
| | | | J = 9.0 Hz, 1H), 7.44 (dd, J = 6.4, 2.0 Hz, 1H), 7.15 (br d, J = 7.8 |
| | | | Hz, 1H), 6.93-6.90 (m, 1H), 6.83 (s, 1H), 6.35-6.31 (m, 1H), |
| | | | 4.28-4.24 (m, 1H), 3.70-3.62 (m, 1H), 3.49-3.42 (m, 1H), 3.20 |
| | | | (br s, 4H), 2.69-2.65 (m, 5H), 2.63-2.56 (m, 2H), 2.34-2.25 |
| | | | (m, 3H), 2.20-2.13 (m, 1H), 2.09-1.99 (m, 3H), 1.87-1.80 (m, |
| | | | 4H), 1.74 (br d, J = 11.3 Hz, 2H), 1.51-1.40 (m, 2H), 1.37-1.24 |
| | | | (m, 5H), 1.23-1.12 (m, 3H) |
| 405 | 811 | 812 | δ (DMSO-d6) 10.79 (s, 1H), 8.47 (d, J = 3.6 Hz, 1H), 8.14 (s, |
| | | | 1H), 8.06 (br d, J = 7.6 Hz, 1H), 7.98 (br s, 1H), 7.82-7.67 (m, |
| | | | 3H), 7.63-7.52 (m, 2H), 7.24 (br d, J = 7.6 Hz, 1H), 6.85 (br t, |
| | | | J = 9.3 Hz, 1H), 6.57-6.48 (m, 2H), 6.44 (br d, J = 8.6 Hz, 1H), |
| | | | 6.37 (t, J = 6.3 Hz, 1H), 5.88 (br d, J = 7.0 Hz, 1H), 5.34-5.09 |
| | | | (m, 1H), 4.33-4.20 (m, 2H), 3.68-3.60 (m, 4H), 2.75-2.66 (m, |
| | | | 6H), 2.59 (br s, 2H), 2.15-2.04 (m, 3H), 1.98 (br d, J = 10.1 Hz, |
| | | | 3H), 1.88-1.78 (m, 5H), 1.36-1.22 (m, 5H) |
| 406 | 811 | 812 | δ (DMSO-d6) 11.86 (br s, 1H), 10.78 (s, 1H), 8.42 (d, J = 3.5 Hz, |
| | | | 1H), 8.35-8.30 (m, 1H), 8.14 (s, 1H), 7.92-7.85 (m, 2H), 7.71 |
| | | | (dd, J = 6.9, 2.0 Hz, 1H), 7.60 (br d, J = 7.8 Hz, 1H), 7.57-7.51 |
| | | | (m, 1H), 7.44 (br d, J = 4.6 Hz, 1H), 7.16 (d, J = 7.8 Hz, 1H), 6.82 |
| | | | (t, J = 9.4 Hz, 1H), 6.51 (dd, J = 15.1, 2.1 Hz, 1H), 6.42 (dd, J = |
| | | | 8.8, 1.8 Hz, 1H), 6.33 (t, J = 6.6 Hz, 1H), 5.82 (d, J = 7.6 Hz, 1H), |
| | | | 5.11-4.91 (m, 1H), 4.26 (ddd, J = 11.6, 7.3, 4.8 Hz, 1H), 3.65 |
| | | | (br d, J = 6.9 Hz, 1H), 3.51-3.48 (m, 1H), 3.20 (br s, 1H), 3.01 |
| | | | (br d, J = 4.0 Hz, 2H), 2.86-2.76 (m, 1H), 2.76-2.69 (m, 2H), |
| | | | 2.68-2.65 (m, 1H), 2.59 (br d, J = 3.9 Hz, 1H), 2.34-2.22 (m, |
| | | | 2H), 2.11-2.03 (m, 2H), 1.98 (br d, J = 8.9 Hz, 3H), 1.91-1.82 |
| | | | (m, 1H), 1.79 (br d, J = 13.6 Hz, 2H), 1.70-1.61 (m, 3H), 1.59- |
| | | | 1.48 (m, 2H), 1.37-1.21 (m, 4H) |
| 407 | 792 | 793 | δ (DMSO-d6) 11.95-11.74 (m, 1H), 10.85-10.71 (m, 1H), 8.43 |
| | | | (d, J = 3.6 Hz, 1H), 8.38-8.30 (m, 1H), 7.95-7.84 (m, 2H), 7.71 |
| | | | (dd, J = 7.0, 2.0 Hz, 1H), 7.56 (t, J = 7.8 Hz, 1H), 7.44 (dd, J = |
| | | | 6.4, 1.9 Hz, 1H), 7.22 (br d, J = 7.6 Hz, 1H), 6.83 (br t, J = 9.3 |
| | | | Hz, 1H), 6.50 (dd, J = 14.9, 2.3 Hz, 1H), 6.42 (dd, J = 8.8, 2.1 Hz, |
| | | | 1H), 6.33 (t, J = 6.6 Hz, 1H), 5.78 (br d, J = 7.5 Hz, 1H), 4.46- |
| | | | 4.36 (m, 1H), 4.32-4.21 (m, 1H), 3.99-3.89 (m, 1H), 3.66 (br |
| | | | dd, J = 5.0, 1.9 Hz, 1H), 3.10 (br d, J = 10.9 Hz, 2H), 3.04-2.93 |
| | | | (m, 1H), 2.73 (dt, J = 11.9, 5.9 Hz, 1H), 2.60-2.56 (m, 2H), 2.14- |
| | | | 2.06 (m, 1H), 2.05-1.97 (m, 2H), 1.85 (dq, J = 12.2, 4.5 Hz, |
| | | | 2H), 1.77-1.67 (m, 6H), 1.65 (br s, 2H), 1.55-1.32 (m, 6H), |
| | | | 1.29-1.22 (m, 2H), 1.18 (br d, J = 6.0 Hz, 2H), 1.07-0.96 (m, |
| | | | 1H), 0.94-0.81 (m, 1H) |
| 408 | 828 | 829 | δ (DMSO-d6) 11.03-10.64 (m, 1H), 8.47-8.40 (m, 1H), 8.38- |
| | | | 8.29 (m, 1H), 7.96-7.84 (m, 2H), 7.75-7.68 (m, 1H), 7.56 (br t, |
| | | | J = 7.3 Hz, 1H), 7.47-7.41 (m, 1H), 7.22 (br d, J = 6.5 Hz, 1H), |
| | | | 7.13-7.05 (m, 1H), 6.54-6.44 (m, 2H), 6.37-6.30 (m, 1H), 6.18 |
| | | | (br d, J = 7.8 Hz, 1H), 4.45-4.30 (m, 2H), 4.01-3.90 (m, 1H), |
| | | | 3.67 (br d, J = 3.3 Hz, 1H), 3.19-3.09 (m, 2H), 3.07-2.89 (m, |
| | | | 3H), 2.81-2.67 (m, 2H), 2.33-2.22 (m, 3H), 2.21-1.96 (m, 6H), |
| | | | 1.79 (br s, 2H), 1.77-1.63 (m, 5H), 1.55-1.28 (m, 5H), 1.07- |
| | | | 0.85 (m, 2H) |
| 409 | 808 | 809 | δ (DMSO-d6) 10.78 (br s, 1H), 8.46 (d, J = 3.6 Hz, 1H), 8.06 (br |
| | | | d, J = 7.5 Hz, 1H), 7.97 (s, 1H), 7.72-7.67 (m, 2H), 7.61-7.57 |
| | | | (m, 1H), 7.54 (ddd, J = 9.1, 6.7, 2.1 Hz, 1H), 7.28 (d, J = 7.6 Hz, |
| | | | 1H), 6.99-6.93 (m, 1H), 6.51 (d, J = 9.3 Hz, 1H), 6.47-6.39 (m, |
| | | | 2H), 6.35 (dt, J = 6.7, 1.3 Hz, 1H), 5.98 (d, J = 7.8 Hz, 1H), 4.29 |
| | | | (ddd, J = 11.8, 7.3, 4.9 Hz, 1H), 4.20 (s, 1H), 4.04 (br d, J = 12.5 |
| | | | Hz, 1H), 3.70-3.61 (m, 2H), 3.05-2.88 (m, 4H), 2.79-2.65 (m, |
| | | | 2H), 2.27 (s, 2H), 2.21 (br t, J = 10.9 Hz, 2H), 2.07 (dt, J = 8.6, |
| | | | 4.6 Hz, 1H), 1.99 (br d, J = 8.9 Hz, 2H), 1.85 (br dd, J = 12.3, 4.3 |
| | | | Hz, 1H), 1.75-1.64 (m, 4H), 1.64-1.54 (m, 4H), 1.50 (br d, J = |
| | | | 9.8 Hz, 2H), 1.44 (br d, J = 13.4 Hz, 2H), 1.39-1.26 (m, 4H) |
| 410 | 809 | 810 | δ (DMSO-d6) 10.82-10.74 (m, 1H), 8.47 (d, J = 3.5 Hz, 1H), |
| | | | 8.14 (s, 1H), 8.07 (br d, J = 7.6 Hz, 1H), 7.98 (s, 1H), 7.75-7.67 |
| | | | (m, 2H), 7.63-7.58 (m, 1H), 7.58-7.51 (m, 1H), 7.29 (br d, J = |
| | | | 7.6 Hz, 1H), 6.90-6.82 (m, 1H), 6.55-6.47 (m, 2H), 6.44-6.39 |
| | | | (m, 1H), 6.38-6.33 (m, 1H), 5.77 (br d, J = 7.6 Hz, 1H), 4.30- |
| | | | 4.21 (m, 1H), 4.20-4.02 (m, 1H), 3.71-3.60 (m, 1H), 3.50 (br |
| | | | d, J = 1.0 Hz, 4H), 2.94-2.80 (m, 4H), 2.79-2.66 (m, 2H), 2.59 |
| | | | (br d, J = 4.4 Hz, 2H), 2.55 (br s, 3H), 2.39-2.28 (m, 2H), 2.14- |
| | | | 2.05 (m, 1H), 2.00 (br d, J = 9.8 Hz, 2H), 1.91-1.79 (m, 1H), |
| | | | 1.71 (br d, J = 10.5 Hz, 4H), 1.58 (br d, J = 11.4 Hz, 2H), 1.52- |
| | | | 1.40 (m, 2H), 1.40-1.28 (m, 2H) |
| 411 | 785 | 786 | δ (DMSO-d6) 10.78 (s, 1H), 8.43 (d, J = 3.6 Hz, 1H), 8.16 (s, |
| | | | 1H), 7.69 (br dd, J = 12.3, 8.3 Hz, 2H), 7.63-7.54 (m, 2H), 7.17 |
| | | | (br d, J = 7.6 Hz, 1H), 6.82 (t, J = 9.3 Hz, 1H), 6.52 (dd, J = 15.1, |
| | | | 2.2 Hz, 1H), 6.45-6.40 (m, 1H), 5.81 (br d, J = 7.5 Hz, 1H), 4.48 |
| | | | (t, J = 7.9 Hz, 2H), 4.30-4.22 (m, 1H), 4.13 (t, J = 7.9 Hz, 2H), |
| | | | 3.68-3.62 (m, 1H), 3.53-3.45 (m, 3H), 2.85 (br s, 3H), 2.79- |
| | | | 2.72 (m, 1H), 2.66 (br s, 4H), 2.35-2.27 (m, 1H), 2.09 (ddd, J = |
| | | | 12.9, 8.7, 4.4 Hz, 1H), 2.02-1.94 (m, 3H), 1.92-1.85 (m, 3H), |
| | | | 1.82-1.74 (m, 4H), 1.42-1.29 (m, 5H), 1.28-1.20 (m, 3H) |
| 412 | 797 | 798 | δ (DMSO-d6) 10.80 (s, 1H), 8.43 (d, J = 3.6 Hz, 1H), 7.87 (s, |
| | | | 1H), 7.84 (br d, J = 7.4 Hz, 1H), 7.69 (br d, J = 7.4 Hz, 1H), 7.61- |
| | | | 7.53 (m, 1H), 7.46 (br d, J = 8.0 Hz, 1H), 7.19 (br d, J = 7.9 Hz, |
| | | | 1H), 6.88 (br t, J = 9.4 Hz, 1H), 6.55 (dd, J = 15.0, 2.1 Hz, 1H), |
| | | | 6.46 (br d, J = 8.8 Hz, 1H), 5.93 (br d, J = 6.6 Hz, 1H), 4.33-4.25 |
| | | | (m, 1H), 3.66 (br t, J = 5.3 Hz, 3H), 3.50 (br d, J = 7.9 Hz, 1H), |
| | | | 3.21-2.93 (m, 6H), 2.80-2.67 (m, 2H), 2.60 (br d, J = 4.4 Hz, |
| | | | 1H), 2.57 (br d, J = 3.8 Hz, 1H), 2.43 (br t, J = 6.3 Hz, 2H), 2.14 |
| | | | (s, 1H), 2.09 (td, J = 8.5, 4.0 Hz, 4H), 2.00-1.94 (m, 2H), 1.91- |
| | | | 1.81 (m, 8H), 1.48-1.35 (m, 5H), 1.34-1.23 (m, 4H) |
| 413 | 798 | 799 | δ (DMSO-d6) 10.80 (s, 1H), 8.42 (d, J = 3.6 Hz, 1H), 7.89-7.79 |
| | | | (m, 2H), 7.59-7.51 (m, 1H), 7.45 (br d, J = 7.9 Hz, 1H), 7.24 (br |
| | | | d, J = 6.4 Hz, 1H), 7.15-6.86 (m, 1H), 6.63-6.42 (m, 2H), 4.37- |
| | | | 4.27 (m, 1H), 3.94-3.87 (m, 1H), 3.80-3.67 (m, 3H), 3.64 (br |
| | | | t, J = 5.4 Hz, 4H), 3.25 (br d, J = 10.5 Hz, 3H), 3.05 (br d, J = 1.1 |
| | | | Hz, 1H), 2.77-2.68 (m, 1H), 2.61-2.54 (m, 2H), 2.41 (br t, J = |
| | | | 6.2 Hz, 2H), 2.10-2.04 (m, 1H), 2.00 (br d, J = 8.6 Hz, 4H), 1.93- |
| | | | 1.81 (m, 7H), 1.71 (br d, J = 11.6 Hz, 4H), 1.53-1.41 (m, 3H), |
| | | | 1.40-1.24 (m, 4H) |
| 414 | 796 | 797 | δ (DMSO-d6) 10.79 (s, 1H), 8.43 (d, J = 3.6 Hz, 1H), 7.90-7.80 |
| | | | (m, 2H), 7.56 (t, J = 7.9 Hz, 1H), 7.46 (br d, J = 7.9 Hz, 1H), 7.29- |
| | | | 7.20 (m, 1H), 7.05-6.77 (m, 1H), 6.58-6.49 (m, 1H), 6.48- |
| | | | 6.40 (m, 1H), 6.13-5.64 (m, 1H), 4.41 (br d, J = 12.8 Hz, 1H), |
| | | | 4.28 (td, J = 7.2, 2.0 Hz, 1H), 3.94 (br d, J = 12.4 Hz, 1H), 3.65 |
| | | | (br t, J = 5.5 Hz, 3H), 3.17 (br s, 2H), 3.00 (br t, J = 12.8 Hz, 1H), |
| | | | 2.80-2.65 (m, 2H), 2.60 (br d, J = 4.0 Hz, 1H), 2.56 (br s, 2H), |
| | | | 2.43 (br t, J = 6.3 Hz, 2H), 2.08 (dt, J = 4.4, 8.7 Hz, 1H), 2.00 (br |
| | | | d, J = 8.9 Hz, 2H), 1.93-1.83 (m, 5H), 1.71 (br d, J = 11.1 Hz, |
| | | | 6H), 1.66-1.55 (m, 2H), 1.53-1.41 (m, 3H), 1.38-1.24 (m, 4H), |
| | | | 1.19 (br d, J = 6.1 Hz, 2H), 1.07-0.96 (m, 1H), 0.89 (br d, J = |
| | | | 11.4 Hz, 1H) |
| 415 | 797 | 798 | δ (DMSO-d6) 10.83-10.72 (m, 1H), 8.46-8.40 (m, 1H), 7.88- |
| | | | 7.82 (m, 2H), 7.58-7.53 (m, 1H), 7.45 (br d, J = 8.3 Hz, 1H), |
| | | | 7.27-7.22 (m, 1H), 6.86-6.79 (m, 1H), 6.53-6.47 (m, 1H), 6.44- |
| | | | 6.39 (m, 1H), 5.80-5.76 (m, 1H), 4.28-4.21 (m, 1H), 3.65 (br |
| | | | t, J = 5.4 Hz, 3H), 3.52-3.43 (m, 4H), 3.14-3.08 (m, 2H), 2.79- |
| | | | 2.67 (m, 1H), 2.55 (br s, 3H), 2.44-2.41 (m, 2H), 2.35 (br d, |
| | | | J = 9.0 Hz, 2H), 2.28 (br s, 2H), 2.18 (br d, J = 6.9 Hz, 2H), 2.13- |
| | | | 2.05 (m, 1H), 2.00 (br d, J = 9.9 Hz, 2H), 1.96-1.81 (m, 6H), |
| | | | 1.79-1.69 (m, 4H), 1.64-1.54 (m, 1H), 1.53-1.41 (m, 2H), 1.41- |
| | | | 1.31 (m, 2H), 1.30-1.20 (m, 2H) |
| 416 | 821 | 822 | δ 10.78 (s, 1H), 8.46 (d, J = 3.6 Hz, 1H), 8.13 (s, 1H), 8.05 (br d, |
| | | | J = 7.4 Hz, 1H), 7.97 (s, 1H), 7.74-7.67 (m, 3H), 7.61-7.51 (m, |
| | | | 2H), 7.22 (d, J = 8.0 Hz, 1H), 6.84 (t, J = 9.3 Hz, 1H), 6.54-6.49 |
| | | | (m, 2H), 6.42 (dd, J = 8.7, 2.1 Hz, 1H), 6.35 (dt, J = 1.3, 6.7 Hz, |
| | | | 1H), 5.84 (br d, J = 7.4 Hz, 1H), 4.30-4.21 (m, 1H), 3.70-3.58 |
| | | | (m, 1H), 2.95-2.86 (m, 6H), 2.78-2.65 (m, 4H), 2.59 (br t, J = |
| | | | 4.0 Hz, 1H), 2.54 (br s, 1H), 2.12-2.05 (m, 2H), 1.99 (br d, J = |
| | | | 10.4 Hz, 2H), 1.90-1.81 (m, 1H), 1.80-1.65 (m, 7H), 1.51-1.39 |
| | | | (m, 3H), 1.33-1.20 (m, 3H), 0.93-0.80 (m, 5H) |
| 417 | 814 | 815 | δ 12.28-11.28 (m, 1H), 10.84 (br s, 1H), 8.42 (d, J = 3.6 Hz, |
| | | | 1H), 8.33 (br s, 1H), 7.95-7.81 (m, 2H), 7.70 (dd, J = 2.0, 7.0 |
| | | | Hz, 1H), 7.55 (t, J = 7.8 Hz, 1H), 7.49 (d, J = 9.0 Hz, 1H), 7.46- |
| | | | 7.39 (m, 1H), 7.20 (br d, J = 7.6 Hz, 1H), 6.91 (dd, J = 1.4, 9.1 |
| | | | Hz, 1H), 6.83 (s, 1H), 6.32 (t, J = 6.7 Hz, 1H), 4.39 (br d, J = 12.5 |
| | | | Hz, 1H), 4.25 (dd, J = 5.1, 9.1 Hz, 1H), 4.02-3.80 (m, 4H), 3.73- |
| | | | 3.59 (m, 1H), 3.24-3.15 (m, 4H), 3.08-2.93 (m, 1H), 2.70- |
| | | | 2.53 (m, 5H), 2.38-2.23 (m, 2H), 2.22-2.09 (m, 3H), 2.06-1.95 |
| | | | (m, 2H), 1.91-1.61 (m, 6H), 1.56-1.27 (m, 5H), 1.10-0.81 (m, |
| | | | 2H) |
| 418 | 792 | 793 | δ (DMSO-d6) 10.84 (br s, 1H), 8.52 (br d, J = 3.1 Hz, 1H), 8.18- |
| | | | 8.00 (m, 2H), 7.81-7.71 (m, 2H), 7.68-7.56 (m, 3H), 7.27 (br |
| | | | d, J = 8.1 Hz, 1H), 7.04 (br t, J = 8.4 Hz, 1H), 6.58 (br d, J = 9.1 |
| | | | Hz, 1H), 6.53-6.46 (m, 2H), 6.42 (br t, J = 6.5 Hz, 1H), 6.04 (br |
| | | | d, J = 7.3 Hz, 1H), 4.40-4.29 (m, 1H), 2.95 (br d, J = 8.9 Hz, |
| | | | 2H), 2.80-2.73 (m, 1H), 2.31 (br t, J = 8.8 Hz, 3H), 2.19-1.99 |
| | | | (m, 5H), 1.93-1.75 (m, 8H), 1.72-1.60 (m, 4H), 1.58-1.42 (m, |
| | | | 3H), 1.38-1.30 (m, 4H), 1.27-1.13 (m, 3H) |
| 419 | 792 | 793 | δ (DMSO-d6) 12.04-11.78 (m, 1H), 10.93-10.75 (m, 1H), 8.52- |
| | | | 8.33 (m, 2H), 8.00-7.88 (m, 2H), 7.79-7.44 (m, 4H), 7.25- |
| | | | 6.97 (m, 2H), 6.56-6.32 (m, 3H), 6.08-5.99 (m, 1H), 4.43-4.27 |
| | | | (m, 1H), 3.04-2.89 (m, 3H), 2.21-1.98 (m, 6H), 1.94-1.45 (m, |
| | | | 16H), 1.40-1.09 (m, 8H) |
| 420 | 793 | 794 | δ (DMSO-d6) 10.78 (s, 1H), 8.45 (br d, J = 2.4 Hz, 1H), 8.06 (br |
| | | | d, J = 6.8 Hz, 1H), 7.97 (br s, 1H), 7.74-7.67 (m, 2H), 7.61- |
| | | | 7.52 (m, 3H), 7.22 (br d, J = 7.7 Hz, 1H), 6.81 (br t, J = 9.2 Hz, |
| | | | 1H), 6.55-6.47 (m, 2H), 6.44-6.30 (m, 2H), 5.80 (br d, J = 6.6 |
| | | | Hz, 1H), 4.25 (br d, J = 4.6 Hz, 1H), 3.64 (br dd, J = 3.4, 1.8 Hz, |
| | | | 1H), 3.48-3.40 (m, 1H), 2.89-2.78 (m, 4H), 2.74-2.57 (m, 6H), |
| | | | 2.36-2.30 (m, 1H), 2.29-2.19 (m, 1H), 2.08-1.95 (m, 4H), 1.82 |
| | | | (br s, 4H), 1.71 (br d, J = 10.6 Hz, 2H), 1.47-1.38 (m, 2H), 1.30- |
| | | | 1.22 (m, 4H), 1.20-1.14 (m, 2H) |
| 421 | 792 | 793 | δ (DMSO-d6) 11.93-11.79 (m, 1H), 10.79 (s, 1H), 8.43 (d, J = |
| | | | 3.6 Hz, 1H), 8.37-8.31 (m, 1H), 7.94-7.85 (m, 2H), 7.73-7.68 |
| | | | (m, 1H), 7.59-7.52 (m, 1H), 7.43 (br d, J = 4.5 Hz, 1H), 7.21 (br |
| | | | d, J = 7.3 Hz, 1H), 7.03-6.95 (m, 1H), 6.48-6.40 (m, 2H), 6.33 |
| | | | (t, J = 6.6 Hz, 1H), 5.99 (d, J = 7.5 Hz, 1H), 4.44-4.35 (m, 1H), |
| | | | 4.31 (ddd, J = 4.7, 7.5, 11.8 Hz, 1H), 3.98-3.90 (m, 1H), 3.71- |
| | | | 3.61 (m, 1H), 3.05-2.95 (m, 1H), 2.92 (br d, J = 10.3 Hz, 2H), |
| | | | 2.70-2.65 (m, 2H), 2.36-2.31 (m, 2H), 2.14 (br d, J = 6.6 Hz, |
| | | | 2H), 2.11-2.06 (m, 1H), 2.04-1.98 (m, 2H), 1.93 (br dd, J = 3.4, |
| | | | 9.3 Hz, 2H), 1.86 (br dd, J = 4.7, 12.9 Hz, 1H), 1.82-1.76 (m, |
| | | | 2H), 1.73 (br s, 2H), 1.69 (br s, 2H), 1.63 (br s, 4H), 1.51-1.42 |
| | | | (m, 2H), 1.36 (br dd, J = 6.6, 8.5 Hz, 2H), 1.05-0.86 (m, 2H) |
| 422 | 813 | 814 | δ (DMSO-d6) 10.88 (s, 1H), 8.47 (d, J = 3.7 Hz, 1H), 8.07 (br d, |
| | | | J = 7.2 Hz, 1H), 7.98 (s, 1H), 7.74-7.67 (m, 2H), 7.61 (s, 1H), |
| | | | 7.59 (s, 1H), 7.55 (ddd, J = 9.1, 6.8, 2.1 Hz, 1H), 7.45 (s, 1H), |
| | | | 7.29 (d, J = 7.6 Hz, 1H), 7.04 (d, J = 8.6 Hz, 1H), 6.52 (d, J = 9.2 |
| | | | Hz, 1H), 6.36 (dt, J = 1.1, 6.7 Hz, 1H), 4.40 (br d, J = 12.0 Hz, |
| | | | 1H), 4.36-4.31 (m, 1H), 3.97 (s, 3H), 3.93 (br d, J = 1.2 Hz, 1H), |
| | | | 3.71-3.61 (m, 1H), 3.06-2.93 (m, 3H), 2.70-2.60 (m, 3H), 2.57 |
| | | | (br d, J = 6.1 Hz, 1H), 2.36-2.30 (m, 1H), 2.21-2.13 (m, 3H), |
| | | | 2.05-1.95 (m, 4H), 1.79 (br s, 6H), 1.71 (br d, J = 12.2 Hz, 3H), |
| | | | 1.51-1.40 (m, 2H), 1.39-1.28 (m, 2H), 1.04 (d, J = 6.1 Hz, 2H), |
| | | | 0.96-0.84 (m, 1H) |
| 423 | 809 | 810 | δ (DMSO-d6) 10.77 (s, 1H), 8.46 (d, J = 3.6 Hz, 1H), 8.11-7.92 |
| | | | (m, 2H), 7.75-7.65 (m, 2H), 7.64-7.43 (m, 2H), 7.27 (br d, J = |
| | | | 7.8 Hz, 1H), 6.81 (t, J = 9.4 Hz, 1H), 6.53-6.47 (m, 2H), 6.41 |
| | | | (dd, J = 8.8, 2.0 Hz, 1H), 6.35 (dt, J = 6.7, 1.2 Hz, 1H), 5.79 (d, |
| | | | J = 7.6 Hz, 1H), 4.29-4.19 (m, 2H), 4.01 (br d, J = 11.9 Hz, 1H), |
| | | | 3.64 (br d, J = 8.5 Hz, 2H), 2.96 (br t, J = 10.3 Hz, 1H), 2.83 (br |
| | | | s, 4H), 2.70-2.58 (m, 5H), 2.34-2.29 (m, 2H), 2.06 (br d, J = |
| | | | 3.9 Hz, 1H), 2.02-1.95 (m, 2H), 1.90-1.78 (m, 1H), 1.69 (br d, |
| | | | J = 11.8 Hz, 2H), 1.57-1.40 (m, 6H), 1.39-1.30 (m, 3H), 1.23 |
| | | | (s, 1H), 1.15 (br s, 1H) |
| 424 | 799 | 800 | δ (DMSO-d6) 10.80 (s, 1H), 8.45 (d, J = 3.5 Hz, 1H), 7.96 (s, |
| | | | 1H), 7.85 (br d, J = 6.1 Hz, 1H), 7.63 (br d, J = 7.9 Hz, 1H), 7.58- |
| | | | 7.53 (m, 2H), 7.22 (br d, J = 7.6 Hz, 1H), 6.82 (t, J = 9.3 Hz, |
| | | | 1H), 6.52 (dd, J = 15.2, 2.2 Hz, 1H), 6.45-6.40 (m, 1H), 5.82 (d, |
| | | | J = 7.6 Hz, 1H), 4.38 (t, J = 5.3 Hz, 2H), 4.31-4.23 (m, 1H), 3.72 |
| | | | (t, J = 6.0 Hz, 2H), 3.65 (br dd, J = 7.3, 1.1 Hz, 1H), 3.54-3.45 |
| | | | (m, 1H), 2.84 (br s, 4H), 2.78-2.68 (m, 1H), 2.61 (br d, J = 4.6 |
| | | | Hz, 4H), 2.31-2.21 (m, 1H), 2.17-2.07 (m, 3H), 2.02-1.93 (m, |
| | | | 3H), 1.91-1.83 (m, 3H), 1.83-1.74 (m, 4H), 1.44-1.32 (m, 4H), |
| | | | 1.31-1.16 (m, 5H) |
| 425 | 798 | 799 | δ (DMSO-d6) 10.80 (s, 1H), 8.45 (d, J = 3.5 Hz, 1H), 7.97 (s, |
| | | | 1H), 7.86 (br d, J = 6.5 Hz, 1H), 7.61-7.51 (m, 2H), 7.27 (br d, |
| | | | J = 7.8 Hz, 1H), 6.90-6.75 (m, 1H), 6.57-6.47 (m, 1H), 6.43 (br |
| | | | d, J = 8.3 Hz, 1H), 5.86-5.74 (m, 1H), 4.42-4.36 (m, 3H), 4.31- |
| | | | 4.22 (m, 1H), 3.94 (br d, J = 13.3 Hz, 1H), 3.72 (t, J = 6.0 Hz, |
| | | | 2H), 3.69-3.61 (m, 1H), 3.17-3.06 (m, 2H), 3.05-2.97 (m, 1H), |
| | | | 2.81-2.68 (m, 2H), 2.60 (br d, J = 3.7 Hz, 2H), 2.18-2.08 (m, |
| | | | 3H), 2.04-1.98 (m, 2H), 1.91-1.84 (m, 1H), 1.71 (br d, J = 11.2 |
| | | | Hz, 6H), 1.67-1.57 (m, 2H), 1.56-1.39 (m, 4H), 1.39-1.32 (m, |
| | | | 2H), 1.31-1.22 (m, 2H), 1.18 (br t, J = 6.4 Hz, 2H), 1.07-0.98 |
| | | | (m, 1H), 0.95-0.84 (m, 1H) |
| 426 | 799 | 800 | δ (DMSO-d6) 10.77 (s, 1H), 8.44 (d, J = 3.6 Hz, 1H), 7.96 (s, |
| | | | 1H), 7.85 (br d, J = 6.1 Hz, 1H), 7.60-7.51 (m, 2H), 7.24 (br d, |
| | | | J = 7.7 Hz, 1H), 6.83 (t, J = 9.4 Hz, 1H), 6.51 (dd, J = 2.4, 15.0 |
| | | | Hz, 1H), 6.42 (dd, J = 2.1, 8.4 Hz, 1H), 5.80 (d, J = 7.6 Hz, 1H), |
| | | | 4.43-4.35 (m, 3H), 4.30-4.21 (m, 1H), 3.94 (br dd, J = 1.8, 12.6 |
| | | | Hz, 1H), 3.75-3.60 (m, 3H), 3.08-2.95 (m, 2H), 2.85 (br s, 4H), |
| | | | 2.80-2.66 (m, 2H), 2.62-2.56 (m, 2H), 2.36-2.30 (m, 1H), 2.20- |
| | | | 2.06 (m, 6H), 2.05-1.95 (m, 2H), 1.92-1.77 (m, 3H), 1.76- |
| | | | 1.66 (m, 4H), 1.52-1.42 (m, 2H), 1.39-1.29 (m, 2H), 1.08-0.97 |
| | | | (m, 1H), 0.95-0.82 (m, 1H) |
| 427 | 798 | 799 | δ (DMSO-d6) 10.79 (s, 1H), 8.44 (d, J = 3.5 Hz, 1H), 7.96 (s, |
| | | | 1H), 7.89-7.82 (m, 1H), 7.60-7.52 (m, 2H), 7.25 (br d, J = 7.5 |
| | | | Hz, 1H), 7.03-6.96 (m, 1H), 6.48-6.41 (m, 2H), 6.00 (d, J = 7.9 |
| | | | Hz, 1H), 4.38 (t, J = 5.3 Hz, 3H), 4.31 (ddd, J = 11.7, 7.5, 4.8 Hz, |
| | | | 1H), 3.94 (br d, J = 12.0 Hz, 1H), 3.72 (t, J = 6.1 Hz, 2H), 3.68- |
| | | | 3.60 (m, 1H), 3.05-2.96 (m, 1H), 2.92 (br d, J = 10.9 Hz, 2H), |
| | | | 2.79 (s, 1H), 2.72-2.67 (m, 1H), 2.59 (br d, J = 3.8 Hz, 2H), 2.38- |
| | | | 2.29 (m, 1H), 2.17-2.12 (m, 4H), 2.11-2.06 (m, 1H), 2.00 (br |
| | | | d, J = 12.0 Hz, 2H), 1.97-1.90 (m, 2H), 1.90-1.84 (m, 1H), 1.82- |
| | | | 1.76 (m, 2H), 1.71 (br d, J = 12.6 Hz, 3H), 1.63 (br s, 4H), 1.52- |
| | | | 1.43 (m, 2H), 1.40-1.32 (m, 2H), 1.07-0.97 (m, 1H), 0.93- |
| | | | 0.84 (m, 1H) |
| 428 | 799 | 800 | δ (DMSO-d6) 10.81-10.74 (m, 1H), 8.44 (d, J = 3.6 Hz, 1H), |
| | | | 7.97-7.95 (m, 1H), 7.86 (br d, J = 6.6 Hz, 1H), 7.59-7.52 (m, |
| | | | 2H), 7.29-7.23 (m, 1H), 6.87-6.80 (m, 1H), 6.53-6.48 (m, 1H), |
| | | | 6.44-6.40 (m, 1H), 5.78 (d, J = 7.8 Hz, 1H), 4.38 (t, J = 5.3 Hz, |
| | | | 2H), 4.30-4.22 (m, 1H), 3.72 (t, J = 5.9 Hz, 2H), 3.68-3.60 (m, |
| | | | 1H), 3.48 (br d, J = 15.5 Hz, 4H), 3.12 (br d, J = 11.8 Hz, 2H), |
| | | | 2.78-2.68 (m, 1H), 2.61-2.57 (m, 2H), 2.37-2.33 (m, 2H), 2.29 |
| | | | (br s, 2H), 2.20-2.16 (m, 2H), 2.16-2.10 (m, 3H), 2.10-2.06 |
| | | | (m, 1H), 2.04-1.97 (m, 2H), 1.91-1.82 (m, 1H), 1.74 (br dd, |
| | | | J = 18.4, 13.9 Hz, 4H), 1.63-1.56 (m, 1H), 1.52-1.42 (m, 2H), |
| | | | 1.40-1.32 (m, 2H), 1.31-1.19 (m, 3H) |
| 429 | 793 | 794 | δ 12.25-11.59 (m, 1H), 10.78 (s, 1H), 9.31-8.99 (m, 1H), 8.42 |
| | | | (br d, J = 3.4 Hz, 1H), 8.31 (s, 1H), 7.86 (br d, J = 7.5 Hz, 3H), |
| | | | 7.70 (br d, J = 6.8 Hz, 1H), 7.55 (br t, J = 7.8 Hz, 1H), 7.43 (br d, |
| | | | J = 5.9 Hz, 1H), 7.17 (br s, 1H), 6.86 (br t, J = 9.2 Hz, 1H), 6.52 |
| | | | (br d, J = 14.8 Hz, 1H), 6.43 (br d, J = 8.6 Hz, 1H), 6.33 (br t, J = |
| | | | 6.6 Hz, 1H), 4.29-4.24 (m, 2H), 3.66 (br s, 2H), 3.56 (br d, J = |
| | | | 11.0 Hz, 3H), 3.26 (br d, J = 10.3 Hz, 3H), 3.04-2.91 (m, 2H), |
| | | | 2.65 (br d, J = 11.9 Hz, 3H), 2.34 (br d, J = 13.0 Hz, 1H), 2.08 (br |
| | | | d, J = 9.8 Hz, 3H), 2.01-1.90 (m, 3H), 1.87-1.71 (m, 5H), 1.56- |
| | | | 1.45 (m, 1H), 1.42-1.24 (m, 4H), 0.93-0.80 (m, 1H) |
| 430 | 793 | 794 | δ 11.86 (br s, 1H), 10.77 (s, 1H), 8.42 (d, J = 3.5 Hz, 1H), 8.33 |
| | | | (br s, 1H), 7.96-7.83 (m, 2H), 7.70 (dd, J = 2.0, 6.9 Hz, 1H), |
| | | | 7.55 (t, J = 7.8 Hz, 1H), 7.43 (br d, J = 5.0 Hz, 1H), 7.22 (br d, |
| | | | J = 7.6 Hz, 1H), 6.83 (t, J = 9.3 Hz, 1H), 6.50 (dd, J = 2.3, 15.0 Hz, |
| | | | 1H), 6.41 (dd, J = 2.1, 8.7 Hz, 1H), 6.33 (t, J = 6.6 Hz, 1H), 5.78 |
| | | | (d, J = 7.6 Hz, 1H), 4.36-4.14 (m, 1H), 3.72-3.60 (m, 1H), 3.47 |
| | | | (br d, J = 14.1 Hz, 4H), 3.11 (br d, J = 11.1 Hz, 2H), 2.82-2.65 |
| | | | (m, 2H), 2.60-2.56 (m, 2H), 2.38-2.26 (m, 4H), 2.18 (br d, J = |
| | | | 6.9 Hz, 2H), 2.12-1.96 (m, 3H), 1.91-1.67 (m, 5H), 1.66-1.08 |
| | | | (m, 8H) |
| 431 | 829 | 830 | δ 10.77 (s, 1H), 8.46 (d, J = 3.6 Hz, 1H), 8.06 (br d, J = 7.6 Hz, |
| | | | 1H), 7.98 (s, 1H), 7.77-7.66 (m, 2H), 7.64-7.49 (m, 3H), 7.21 |
| | | | (br d, J = 7.9 Hz, 1H), 6.85 (t, J = 9.3 Hz, 1H), 6.57-6.47 (m, |
| | | | 2H), 6.42 (dd, J = 1.9, 8.8 Hz, 1H), 6.36 (dt, J = 1.2, 6.7 Hz, 1H), |
| | | | 5.88 (d, J = 7.6 Hz, 1H), 4.33-4.21 (m, 1H), 3.72-3.59 (m, 1H), |
| | | | 3.55-3.42 (m, 2H), 3.28-3.14 (m, 2H), 3.03-2.83 (m, 4H), 2.79- |
| | | | 2.66 (m, 2H), 2.63-2.56 (m, 2H), 2.14-1.95 (m, 4H), 1.95- |
| | | | 1.78 (m, 3H), 1.72 (br t, J = 12.6 Hz, 4H), 1.54-1.18 (m, 6H) |
| 432 | 793 | 794 | δ (DMSO-d6) 11.87 (br s, 1H), 10.79 (s, 1H), 8.42 (d, J = 3.4 Hz, |
| | | | 1H), 8.35-8.28 (m, 1H), 7.90-7.85 (m, 2H), 7.70 (dd, J = 6.9, |
| | | | 1.9 Hz, 1H), 7.63-7.59 (m, 1H), 7.57-7.53 (m, 1H), 7.43 (br d, |
| | | | J = 6.5 Hz, 1H), 7.16 (br d, J = 7.3 Hz, 1H), 6.82 (br t, J = 9.4 Hz, |
| | | | 1H), 6.55-6.46 (m, 1H), 6.42 (br d, J = 8.3 Hz, 1H), 6.32 (t, J = |
| | | | 6.7 Hz, 1H), 5.83 (br dd, J = 5.0, 2.4 Hz, 1H), 4.29-4.20 (m, 1H), |
| | | | 3.72-3.57 (m, 2H), 2.92-2.81 (m, 4H), 2.74-2.65 (m, 3H), 2.60- |
| | | | 2.57 (m, 1H), 2.11-1.95 (m, 6H), 1.86-1.80 (m, 4H), 1.74- |
| | | | 1.70 (m, 2H), 1.47-1.41 (m, 2H), 1.31-1.23 (m, 4H), 1.16 (d, |
| | | | J = 6.6 Hz, 4H) |
| 433 | 807 | 808 | δ 10.76 (s, 1H), 8.45 (d, J = 3.6 Hz, 1H), 8.04 (d, J = 7.0 Hz, 1H), |
| | | | 7.96 (s, 1H), 7.74-7.66 (m, 2H), 7.61-7.50 (m, 3H), 7.21 (br d, |
| | | | J = 7.8 Hz, 1H), 6.81 (t, J = 9.3 Hz, 1H), 6.53-6.45 (m, 2H), 6.43- |
| | | | 6.31 (m, 2H), 5.76 (d, J = 7.5 Hz, 1H), 4.24 (ddd, J = 11.4, 7.2, |
| | | | 4.8 Hz, 1H), 3.63 (br d, J = 7.5 Hz, 1H), 3.53-3.44 (m, 1H), 3.09 |
| | | | (br d, J = 11.0 Hz, 2H), 2.83 (br d, J = 10.4 Hz, 2H), 2.77-2.69 |
| | | | (m, 1H), 2.58 (br s, 1H), 2.13-1.92 (m, 6H), 1.89-1.69 (m, 8H), |
| | | | 1.61-1.51 (m, 5H), 1.39-1.09 (m, 7H) |
| 434 | 792 | 793 | δ (DMSO-d6) 10.84 (br s, 1H), 8.52 (br d, J = 3.1 Hz, 1H), 8.18- |
| | | | 8.00 (m, 2H), 7.81-7.71 (m, 2H), 7.68-7.56 (m, 3H), 7.27 (br |
| | | | d, J = 8.1 Hz, 1H), 7.04 (br t, J = 8.4 Hz, 1H), 6.58 (br d, J = 9.1 |
| | | | Hz, 1H), 6.53-6.46 (m, 2H), 6.42 (br t, J = 6.5 Hz, 1H), 6.04 (br |
| | | | d, J = 7.3 Hz, 1H), 4.40-4.29 (m, 1H), 2.95 (br d, J = 8.9 Hz, |
| | | | 2H), 2.80-2.73 (m, 1H), 2.31 (br t, J = 8.8 Hz, 3H), 2.19-1.99 |
| | | | (m, 5H), 1.93-1.75 (m, 8H), 1.72-1.60 (m, 4H), 1.58-1.42 (m, |
| | | | 3H), 1.38-1.30 (m, 4H), 1.27-1.13 (m, 3H) |
| 435 | 792 | 793 | δ (DMSO-d6) 12.04-11.78 (m, 1H), 10.93-10.75 (m, 1H), 8.52- |
| | | | 8.33 (m, 2H), 8.00-7.88 (m, 2H), 7.79-7.44 (m, 4H), 7.25- |
| | | | 6.97 (m, 2H), 6.56-6.32 (m, 3H), 6.08-5.99 (m, 1H), 4.43-4.27 |
| | | | (m, 1H), 3.04-2.89 (m, 3H), 2.21-1.98 (m, 6H), 1.94-1.45 (m, |
| | | | 16H), 1.40-1.09 (m, 8H) |
| 436 | 793 | 794 | δ (DMSO-d6) 10.77 (s, 1H), 8.45 (br d, J = 2.8 Hz, 1H), 8.08- |
| | | | 7.92 (m, 2H), 7.75-7.65 (m, 2H), 7.61-7.52 (m, 3H), 7.21 (br |
| | | | d, J = 8.0 Hz, 1H), 6.81 (br t, J = 9.2 Hz, 1H), 6.52-6.30 (m, 4H), |
| | | | 5.79 (br d, J = 7.5 Hz, 1H), 4.24 (br d, J = 4.9 Hz, 1H), 3.64 (br |
| | | | dd, J = 4.3, 2.3 Hz, 1H), 3.43 (br dd, J = 6.2, 4.6 Hz, 1H), 2.83 |
| | | | (br s, 4H), 2.60 (br s, 4H), 2.33 (br s, 1H), 2.28-2.19 (m, 1H), |
| | | | 2.08-1.96 (m, 4H), 1.81 (br d, J = 8.1 Hz, 4H), 1.71 (br d, J = |
| | | | 10.3 Hz, 2H), 1.48-1.39 (m, 2H), 1.29-1.23 (m, 4H), 1.21-1.10 |
| | | | (m, 4H) |
| 437 | 792 | 793 | δ (DMSO-d6) 12.14-11.54 (m, 1H), 10.90-10.61 (m, 1H), 8.43 |
| | | | (d, J = 3.5 Hz, 1H), 8.34 (br s, 1H), 7.93-7.89 (m, 1H), 7.87 (br |
| | | | d, J = 7.8 Hz, 1H), 7.71 (dd, J = 2.1, 6.9 Hz, 1H), 7.56 (t, J = 7.9 |
| | | | Hz, 1H), 7.43 (dd, J = 1.9, 6.4 Hz, 1H), 7.24-7.19 (m, 1H), 7.02- |
| | | | 6.96 (m, 1H), 6.51-6.41 (m, 2H), 6.33 (t, J = 6.7 Hz, 1H), 6.00 |
| | | | (d, J = 7.9 Hz, 1H), 4.43-4.36 (m, 1H), 4.35-4.27 (m, 1H), 3.98- |
| | | | 3.90 (m, 1H), 3.72-3.62 (m, 1H), 3.04-2.97 (m, 1H), 2.92 (br |
| | | | d, J = 9.9 Hz, 2H), 2.80-2.69 (m, 3H), 2.14 (br d, J = 6.1 Hz, |
| | | | 2H), 2.08 (br dd, J = 3.7, 8.6 Hz, 1H), 2.02 (br d, J = 10.3 Hz, |
| | | | 2H), 1.97-1.92 (m, 2H), 1.86 (br dd, J = 4.4, 12.3 Hz, 1H), 1.81- |
| | | | 1.75 (m, 2H), 1.71 (br d, J = 13.0 Hz, 3H), 1.63 (br s, 4H), 1.51- |
| | | | 1.43 (m, 2H), 1.39-1.32 (m, 2H), 1.16-1.11 (m, 1H), 1.04 (d, |
| | | | J = 6.0 Hz, 1H), 1.02-0.95 (m, 1H), 0.92-0.85 (m, 1H) |
| 438 | 813 | 814 | δ (DMSO-d6) 10.94 (s, 1H), 8.53 (d, J = 3.5 Hz, 1H), 8.12 (br d, |
| | | | J = 7.8 Hz, 1H), 8.04 (s, 1H), 7.81-7.73 (m, 2H), 7.66 (d, J = 8.3 |
| | | | Hz, 2H), 7.61 (ddd, J = 9.1, 6.8, 2.0 Hz, 1H), 7.50 (s, 1H), 7.35 |
| | | | (d, J = 7.6 Hz, 1H), 7.10 (d, J = 7.9 Hz, 1H), 6.58 (d, J = 9.0 Hz, |
| | | | 1H), 6.41 (dt, J = 1.2, 6.8 Hz, 1H), 4.46 (br d, J = 11.9 Hz, 1H), |
| | | | 4.38 (dd, J = 9.7, 5.1 Hz, 1H), 4.03 (s, 3H), 3.99 (br s, 1H), 3.76- |
| | | | 3.67 (m, 1H), 3.11-2.98 (m, 3H), 2.75-2.64 (m, 4H), 2.42- |
| | | | 2.38 (m, 1H), 2.26-2.18 (m, 3H), 2.10-2.01 (m, 4H), 1.85 (br |
| | | | s, 6H), 1.76 (br d, J = 13.3 Hz, 3H), 1.60-1.47 (m, 2H), 1.46- |
| | | | 1.34 (m, 2H), 1.13-1.05 (m, 2H), 1.01-0.91 (m, 1H) |
| 439 | 809 | 810 | δ (DMSO-d6) 10.76 (s, 1H), 8.45 (d, J = 3.6 Hz, 1H), 8.10-7.91 |
| | | | (m, 2H), 7.76-7.65 (m, 2H), 7.61-7.49 (m, 2H), 7.27 (d, J = 7.6 |
| | | | Hz, 1H), 6.81 (t, J = 9.3 Hz, 1H), 6.54-6.45 (m, 2H), 6.44-6.31 |
| | | | (m, 2H), 5.78 (br d, J = 7.6 Hz, 1H), 4.29-4.20 (m, 2H), 4.06- |
| | | | 3.88 (m, 1H), 3.69-3.59 (m, 2H), 3.01-2.90 (m, 1H), 2.82 (br |
| | | | s, 4H), 2.70-2.56 (m, 5H), 2.34-2.27 (m, 3H), 2.07 (td, J = 8.6, |
| | | | 4.2 Hz, 1H), 1.98 (br d, J = 9.8 Hz, 2H), 1.89-1.77 (m, 1H), 1.69 |
| | | | (br d, J = 11.1 Hz, 2H), 1.57-1.40 (m, 6H), 1.39-1.29 (m, 3H), |
| | | | 1.23 (s, 1H) |
| 440 | 793 | 794 | δ 8.47 (d, J = 3.5 Hz, 1H), 8.06 (br d, J = 7.2 Hz, 1H), 7.98 (br s, |
| | | | 1H), 7.77-7.68 (m, 2H), 7.64-7.60 (m, 2H), 7.56 (ddd, J = 9.1, |
| | | | 6.7, 2.0 Hz, 1H), 7.24 (br d, J = 7.6 Hz, 1H), 6.88-6.78 (m, 1H), |
| | | | 6.55-6.47 (m, 2H), 6.44-6.34 (m, 2H), 5.80 (d, J = 7.6 Hz, 1H), |
| | | | 4.26 (ddd, J = 11.7, 7.0, 4.9 Hz, 1H), 3.75-3.60 (m, 1H), 3.52- |
| | | | 3.44 (m, 1H), 3.16 (br d, J = 11.2 Hz, 2H), 2.87-2.79 (m, 2H), |
| | | | 2.78-2.70 (m, 1H), 2.61-2.57 (m, 1H), 2.33-2.26 (m, 1H), 2.23- |
| | | | 2.16 (m, 2H), 2.13-2.05 (m, 2H), 2.03-1.96 (m, 2H), 1.91- |
| | | | 1.82 (m, 1H), 1.80-1.67 (m, 7H), 1.60-1.53 (m, 2H), 1.49-1.41 |
| | | | (m, 2H), 1.41-1.21 (m, 5H) |
| 441 | 793 | 794 | δ (DMSO-d6) 11.85 (br s, 1H), 10.76 (s, 1H), 8.45-8.26 (m, 2H), |
| | | | 7.94-7.81 (m, 2H), 7.70 (dd, J = 6.9, 2.0 Hz, 1H), 7.59-7.52 |
| | | | (m, 2H), 7.43 (br d, J = 5.8 Hz, 1H), 7.14 (br d, J = 7.8 Hz, 1H), |
| | | | 6.85-6.76 (m, 1H), 6.50 (dd, J = 14.9, 2.2 Hz, 1H), 6.44-6.38 |
| | | | (m, 1H), 6.32 (t, J = 6.6 Hz, 1H), 5.78 (br d, J = 7.6 Hz, 1H), 4.29- |
| | | | 4.18 (m, 1H), 3.71-3.60 (m, 1H), 3.43 (br d, J = 8.8 Hz, 1H), |
| | | | 2.82 (br s, 3H), 2.73-2.66 (m, 2H), 2.59 (br d, J = 3.6 Hz, 4H), |
| | | | 2.54 (s, 1H), 2.33-2.32 (m, 1H), 2.27-2.18 (m, 1H), 2.10-1.97 |
| | | | (m, 4H), 1.81 (br d, J = 8.6 Hz, 4H), 1.72 (br d, J = 11.6 Hz, 2H), |
| | | | 1.49-1.40 (m, 2H), 1.29-1.23 (m, 4H), 1.18-1.13 (m, 2H) |
| 442 | 808 | 809 | δ (DMSO-d6) 10.78 (s, 1H), 8.47 (d, J = 3.5 Hz, 1H), 8.07 (br d, |
| | | | J = 7.6 Hz, 1H), 7.98 (s, 1H), 7.73-7.67 (m, 2H), 7.62-7.51 (m, |
| | | | 2H), 7.30-7.24 (m, 1H), 7.02-6.93 (m, 1H), 6.52 (d, J = 9.3 Hz, |
| | | | 1H), 6.49-6.39 (m, 2H), 6.38-6.33 (m, 1H), 5.99 (br d, J = 7.5 |
| | | | Hz, 1H), 4.35-4.13 (m, 2H), 4.05 (br d, J = 12.6 Hz, 1H), 3.67 |
| | | | (br d, J = 11.8 Hz, 2H), 3.07-2.89 (m, 3H), 2.79-2.67 (m, 1H), |
| | | | 2.61-2.55 (m, 2H), 2.35-2.17 (m, 4H), 2.11-2.05 (m, 1H), 2.00 |
| | | | (br d, J = 9.9 Hz, 2H), 1.91-1.79 (m, 1H), 1.71 (br d, J = 12.4 |
| | | | Hz, 3H), 1.61 (br s, 3H), 1.55-1.23 (m, 10H) |
| 443 | 808 | 809 | δ (DMSO-d6) 10.77 (s, 1H), 8.46 (d, J = 3.5 Hz, 1H), 8.06 (br d, |
| | | | J = 7.9 Hz, 1H), 7.97 (s, 1H), 7.74-7.66 (m, 2H), 7.62-7.50 (m, |
| | | | 2H), 7.27 (d, J = 7.6 Hz, 1H), 7.03-6.93 (m, 1H), 6.51 (d, J = 9.1 |
| | | | Hz, 1H), 6.47-6.39 (m, 2H), 6.35 (dt, J = 6.7, 1.3 Hz, 1H), 5.98 |
| | | | (d, J = 7.6 Hz, 1H), 4.33-4.26 (m, 1H), 4.20 (br s, 1H), 4.04 (br |
| | | | d, J = 12.4 Hz, 1H), 3.71-3.56 (m, 2H), 3.06-2.90 (m, 3H), 2.78- |
| | | | 2.66 (m, 1H), 2.61-2.55 (m, 2H), 2.34-2.18 (m, 4H), 2.07 (qd, |
| | | | J = 12.9, 4.2 Hz, 1H), 1.99 (br d, J = 9.6 Hz, 2H), 1.85 (dq, J = |
| | | | 12.4, 4.9 Hz, 1H), 1.75-1.66 (m, 3H), 1.65-1.54 (m, 4H), 1.54- |
| | | | 1.25 (m, 9H) |
| 444 | 809 | 810 | δ (DMSO-d6) 10.81 (br dd, J = 7.5, 3.1Hz, 1H), 8.47 (d, J = 3.4 |
| | | | Hz, 1H), 8.06 (br d, J = 7.6 Hz, 1H), 7.98 (br s, 1H), 7.74-7.68 |
| | | | (m, 2H), 7.61 (dd, J = 7.9, 0.9 Hz, 1H), 7.56 (ddd, J = 9.1, 6.8, |
| | | | 2.0 Hz, 1H), 7.47 (br d, J = 8.3 Hz, 1H), 7.25 (br d, J = 7.8 Hz, |
| | | | 1H), 6.84 (br t, J = 9.4 Hz, 1H), 6.53 (br d, J = 8.9 Hz, 2H), 6.42- |
| | | | 6.35 (m, 2H), 5.82-5.77 (m, 1H), 5.14 (br s, 1H), 4.31-4.23 |
| | | | (m, 1H), 3.70-3.62 (m, 1H), 3.55-3.50 (m, 1H), 3.17 (br d, J = |
| | | | 9.7 Hz, 3H), 2.77-2.72 (m, 1H), 2.67 (br d, J = 10.1 Hz, 3H), |
| | | | 2.36-2.28 (m, 2H), 2.13-2.05 (m, 2H), 2.00-1.93 (m, 3H), 1.89 |
| | | | (br d, J = 12.6 Hz, 2H), 1.77 (br t, J = 10.6 Hz, 4H), 1.55 (br s, |
| | | | 3H), 1.43-1.33 (m, 6H) |
| 445 | 797 | 798 | δ (DMSO-d6) 10.79 (s, 1H), 8.43 (br d, J = 3.4 Hz, 1H), 7.89- |
| | | | 7.81 (m, 2H), 7.63-7.53 (m, 2H), 7.46 (br d, J = 7.5 Hz, 1H), |
| | | | 7.19 (br d, J = 7.6 Hz, 1H), 6.82 (br t, J = 9.2 Hz, 1H), 6.55-6.48 |
| | | | (m, 1H), 6.42 (br d, J = 7.8 Hz, 1H), 5.80 (br d, J = 7.6 Hz, 1H), |
| | | | 4.30-4.22 (m, 1H), 3.65 (br d, J = 5.5 Hz, 3H), 3.54-3.45 (m, |
| | | | 1H), 2.94 (br s, 1H), 2.84 (br s, 4H), 2.78-2.69 (m, 1H), 2.62 (br |
| | | | s, 4H), 2.43 (br t, J = 6.1 Hz, 2H), 2.27 (br t, J = 10.6 Hz, 1H), |
| | | | 2.13-2.05 (m, 1H), 1.97 (br s, 3H), 1.92-1.83 (m, 8H), 1.82- |
| | | | 1.74 (m, 4H), 1.39-1.30 (m, 4H), 1.28-1.21 (m, 3H) |
| 446 | 784 | 785 | δ (DMSO-d6) 10.81 (s, 1H), 8.44 (d, J = 3.5 Hz, 1H), 8.20 (s, |
| | | | 1H), 7.73 (br s, 2H), 7.63 (br d, J = 8.1 Hz, 1H), 7.55 (s, 1H), 7.22- |
| | | | 7.17 (m, 1H), 7.03-6.95 (m, 1H), 6.48-6.41 (m, 2H), 6.02 (d, |
| | | | J = 7.8 Hz, 1H), 4.49 (t, J = 7.9 Hz, 2H), 4.35-4.28 (m, 1H), 4.13 |
| | | | (t, J = 7.9 Hz, 2H), 3.71-3.61 (m, 2H), 2.96 (br d, J = 9.3 Hz, |
| | | | 2H), 2.78-2.68 (m, 2H), 2.61-2.59 (m, 1H), 2.39 (br dd, J = 8.1, |
| | | | 1.0 Hz, 2H), 2.34 (br d, J = 1.7 Hz, 1H), 2.13-2.06 (m, 1H), 1.99 |
| | | | (br dd, J = 8.3, 2.8 Hz, 3H), 1.90-1.83 (m, 3H), 1.78 (br t, J = |
| | | | 11.2 Hz, 4H), 1.70-1.59 (m, 4H), 1.43-1.33 (m, 4H), 1.31-1.24 |
| | | | (m, 4H) |
| 447 | 796 | 797 | δ (DMSO-d6) 10.80 (s, 1H), 8.43 (d, J = 3.6 Hz, 1H), 8.20 (s, |
| | | | 1H), 7.89-7.81 (m, 2H), 7.62 (br d, J = 7.6 Hz, 1H), 7.58-7.53 |
| | | | (m, 1H), 7.46 (br d, J = 7.6 Hz, 1H), 7.20 (br d, J = 7.4 Hz, 1H), |
| | | | 6.98 (br t, J = 8.9 Hz, 1H), 6.49-6.40 (m, 2H), 6.02 (br d, J = 8.1 |
| | | | Hz, 1H), 4.35-4.27 (m, 1H), 3.67-3.63 (m, 4H), 2.98 (br d, J = |
| | | | 9.4 Hz, 2H), 2.79-2.70 (m, 1H), 2.65-2.58 (m, 2H), 2.43 (br t, |
| | | | J = 6.1 Hz, 5H), 2.09 (td, J =8.6, 4.1 Hz, 1H), 2.01-1.94 (m, 3H), |
| | | | 1.88 (br d, J = 4.0 Hz, 4H), 1.86 (br s, 2H), 1.81-1.74 (m, 4H), |
| | | | 1.67 (br s, 4H), 1.42-1.22 (m, 9H) |
| 448 | 796 | 797 | δ (DMSO-d6) 10.84-10.74 (m, 1H), 8.47-8.41 (m, 1H), 7.89- |
| | | | 7.83 (m, 2H), 7.59-7.53 (m, 1H), 7.48-7.44 (m, 1H), 7.27-7.24 |
| | | | (m, 1H), 7.03-6.95 (m, 1H), 6.49-6.41 (m, 2H), 6.02-5.98 (m, |
| | | | 1H), 4.44-4.36 (m, 1H), 4.34-4.27 (m, 1H), 3.98-3.91 (m, 1H), |
| | | | 3.70-3.60 (m, 3H), 3.05-2.96 (m, 1H), 2.94-2.88 (m, 2H), 2.79- |
| | | | 2.65 (m, 2H), 2.59 (br d, J = 3.6 Hz, 2H), 2.45-2.41 (m, 3H), |
| | | | 2.36-2.32 (m, 1H), 2.15-2.06 (m, 3H), 2.03-1.97 (m, 2H), 1.96- |
| | | | 1.83 (m, 7H), 1.82-1.75 (m, 2H), 1.71 (br dd, J = 2.4, 10.8 Hz, |
| | | | 3H), 1.63 (br s, 3H), 1.53-1.42 (m, 2H), 1.40-1.29 (m, 2H), |
| | | | 1.06-0.95 (m, 1H), 0.93-0.83 (m, 1H) |
| 449 | 796 | 797 | δ (DMSO-d6) 10.80 (s, 1H), 8.44 (d, J = 3.5 Hz, 1H), 7.90-7.81 |
| | | | (m, 2H), 7.59-7.53 (m, 1H), 7.46 (br d, J = 7.1 Hz, 1H), 7.28- |
| | | | 7.23 (m, 1H), 7.03-6.96 (m, 1H), 6.48-6.41 (m, 2H), 6.00 (br |
| | | | d, J = 7.9 Hz, 1H), 4.44-4.36 (m, 1H), 4.32 (br dd, J = 5.5, 2.8 |
| | | | Hz, 1H), 3.97-3.92 (m, 1H), 3.67-3.64 (m, 2H), 3.04-2.97 (m, |
| | | | 3H), 2.94-2.89 (m, 4H), 2.36-2.32 (m, 3H), 2.16-2.06 (m, 4H), |
| | | | 2.01-1.97 (m, 2H), 1.93-1.84 (m, 7H), 1.81-1.76 (m, 2H), 1.74- |
| | | | 1.69 (m, 3H), 1.66-1.62 (m, 4H), 1.51-1.43 (m, 2H), 1.39- |
| | | | 1.31 (m, 2H), 1.07-0.96 (m, 1H), 0.92-0.82 (m, 1H) |
| 450 | 793 | 794 | δ 12.13-11.71 (m, 1H), 10.78 (s, 1H), 8.42 (d, J = 3.5 Hz, 1H), |
| | | | 8.31 (s, 1H), 8.15 (s, 0.38H), 7.87 (br t, J = 7.2 Hz, 2H), 7.70 (dd, |
| | | | J = 2.0, 6.9 Hz, 1H), 7.61 (br d, J = 7.9 Hz, 1H), 7.57-7.49 (m, |
| | | | 1H), 7.43 (dd, J = 1.8, 6.4 Hz, 1H), 7.16 (br d, J = 7.9 Hz, 1H), |
| | | | 6.81 (t, J = 9.4 Hz, 1H), 6.50 (dd, J = 2.0, 15.6 Hz, 1H), 6.45- |
| | | | 6.38 (m, 1H), 6.33 (t, J = 6.7 Hz, 1H), 5.80 (br d, J = 6.0 Hz, 1H), |
| | | | 4.34-4.18 (m, 1H), 3.72-3.58 (m, 3H), 2.85 (br s, 4H), 2.70- |
| | | | 2.62 (m, 5H), 2.38-2.25 (m, 1H), 2.08 (td, J = 4.2, 8.6 Hz, 1H), |
| | | | 2.03-1.66 (m, 9H), 1.48-1.07 (m, 9H), 0.92-0.75 (m, 1H) |
| 451 | 829 | 830 | δ 11.86 (br s, 1H), 10.77 (s, 1H), 8.42 (d, J = 3.6 Hz, 1H), 8.32 |
| | | | (br s, 1H), 7.94-7.83 (m, 2H), 7.70 (dd, J = 2.0, 6.9 Hz, 1H), |
| | | | 7.64-7.52 (m, 2H), 7.43 (br d, J = 5.0 Hz, 1H), 7.15 (br d, J = |
| | | | 7.8 Hz, 1H), 6.84 (t, J = 9.3 Hz, 1H), 6.51 (dd, J = 2.3, 15.0 Hz, |
| | | | 1H), 6.41 (dd, J = 1.5, 8.8 Hz, 1H), 6.32 (t, J = 6.7 Hz, 1H), 5.88 |
| | | | (d, J = 7.5 Hz, 1H), 4.32-4.22 (m, 1H), 3.72-3.59 (m, 1H), 3.55- |
| | | | 3.44 (m, 1H), 3.21 (br s, 2H), 3.01-2.82 (m, 4H), 2.78-2.64 |
| | | | (m, 3H), 2.58 (br d, J = 3.9 Hz, 1H), 2.35-2.30 (m, 1H), 2.11- |
| | | | 1.95 (m, 4H), 1.94-1.78 (m, 3H), 1.77-1.62 (m, 4H), 1.45 (br |
| | | | d, J = 12.5 Hz, 2H), 1.37-1.20 (m, 4H) |
| 452 | 811 | 812 | δ 10.77 (s, 1H), 8.46 (d, J = 3.6 Hz, 1H), 8.06 (br d, J = 7.1 Hz, |
| | | | 1H), 7.97 (s, 1H), 7.75-7.67 (m, 2H), 7.62-7.49 (m, 3H), 7.21 |
| | | | (br d, J = 8.0 Hz, 1H), 6.83 (br s, 1H), 6.54-6.47 (m, 2H), 6.42 |
| | | | (br d, J = 9.0 Hz, 1H), 6.35 (dt, J = 1.2, 6.7 Hz, 1H), 5.88-5.77 |
| | | | (m, 1H), 4.26 (br dd, J = 2.9, 7.6 Hz, 1H), 3.72-3.58 (m, 2H), |
| | | | 3.26-3.19 (m, 2H), 2.80-2.64 (m, 4H), 2.58 (br d, J = 4.1 Hz, |
| | | | 1H), 2.36-2.29 (m, 2H), 2.07 (dt, J = 4.6, 8.4 Hz, 3H), 2.02- |
| | | | 1.95 (m, 4H), 1.85 (br dd, J = 4.4, 12.3 Hz, 2H), 1.74 (br d, J = |
| | | | 11.8 Hz, 4H), 1.52-1.36 (m, 4H), 1.32-1.18 (m, 3H) |
| 453 | 811 | 812 | δ 12.25-11.47 (m, 1H), 11.09-10.51 (m, 1H), 8.42 (d, J = 3.6 |
| | | | Hz, 1H), 8.33 (br s, 1H), 7.95-7.83 (m, 2H), 7.71 (dd, J = 2.0, |
| | | | 6.9 Hz, 1H), 7.62 (br d, J = 7.6 Hz, 1H), 7.56 (t, J = 7.8 Hz, 1H), |
| | | | 7.43 (dd, J = 2.0, 6.4 Hz, 1H), 7.15 (br d, J = 7.9 Hz, 1H), 6.82 (t, |
| | | | J = 9.3 Hz, 1H), 6.51 (dd, J = 2.3, 15.1 Hz, 1H), 6.42 (dd, J = 2.0, |
| | | | 8.8 Hz, 1H), 6.33 (t, J = 6.7 Hz, 1H), 5.82 (d, J = 7.6 Hz, 1H), |
| | | | 5.18-4.80 (m, 1H), 4.26 (ddd, J = 4.9, 7.3, 11.6 Hz, 1H), 3.77- |
| | | | 3.58 (m, 1H), 3.56-3.45 (m, 1H), 3.21 (br d, J = 10.8 Hz, 1H), |
| | | | 3.02-2.87 (m, 2H), 2.85-2.54 (m, 5H), 2.43-2.23 (m, 3H), 2.16- |
| | | | 1.93 (m, 5H), 1.92-1.79 (m, 1H), 1.78-1.60 (m, 5H), 1.54- |
| | | | 1.19 (m, 6H), 0.95 (d, J = 6.6 Hz, 1H). |
| 454 | 792 | 793 | δ 12.05-11.68 (m, 1H), 10.88-10.64 (m, 1H), 8.42 (br d, J = 3.4 |
| | | | Hz, 1H), 8.31 (br s, 1H), 7.97-7.79 (m, 2H), 7.76-7.65 (m, 1H), |
| | | | 7.63-7.49 (m, 2H), 7.43 (br d, J = 6.1 Hz, 1H), 7.15 (br d, J = |
| | | | 7.3 Hz, 1H), 7.03-6.92 (m, 1H), 6.49-6.39 (m, 2H), 6.33 (br t, |
| | | | J = 6.6 Hz, 1H), 5.98 (br d, J = 7.1 Hz, 1H), 4.36-4.22 (m, 3H), |
| | | | 2.89 (br d, J = 7.5 Hz, 3H), 2.76-2.65 (m, 4H), 2.34-2.23 (m, |
| | | | 3H), 2.12-2.04 (m, 1H), 1.96 (br d, J = 7.6 Hz, 3H), 1.88-1.71 |
| | | | (m, 6H), 1.66-1.54 (m, 3H), 1.41-1.15 (m, 8H) |
| 455 | 792 | 793 | δ (DMSO-d6) 10.81 (s, 1H), 8.47 (br d, J = 2.1 Hz, 1H), 8.07 (br |
| | | | d, J = 6.6 Hz, 1H), 7.98 (br s, 1H), 7.76-7.67 (m, 2H), 7.63- |
| | | | 7.52 (m, 2H), 7.31 (br d, J = 7.2 Hz, 1H), 6.98 (br t, J = 8.3 Hz, |
| | | | 1H), 6.55-6.42 (m, 3H), 6.36 (br t, J = 6.7 Hz, 1H), 6.07 (br d, |
| | | | J = 7.7 Hz, 1H), 4.44-4.36 (m, 1H), 4.35-4.29 (m, 1H), 3.96 (br |
| | | | d, J = 11.9 Hz, 1H), 3.71-3.60 (m, 1H), 3.34 (br s, 4H), 3.07- |
| | | | 2.96 (m, 1H), 2.79-2.70 (m, 2H), 2.60 (br d, J = 2.6 Hz, 2H), |
| | | | 2.56 (br s, 2H), 2.12-2.04 (m, 1H), 2.02-1.96 (m, 2H), 1.94- |
| | | | 1.85 (m, 2H), 1.80 (br d, J = 3.3 Hz, 1H), 1.79-1.70 (m, 6H), |
| | | | 1.69 (br s, 1H), 1.52-1.42 (m, 2H), 1.40-1.21 (m, 3H), 1.12- |
| | | | 0.91 (m, 2H) |
| 456 | 792 | 793 | δ (DMSO-d6) 10.81 (s, 1H), 8.47 (br d, J = 2.1 Hz, 1H), 8.07 (br |
| | | | d, J = 6.6 Hz, 1H), 7.98 (br s, 1H), 7.76-7.67 (m, 2H), 7.63- |
| | | | 7.52 (m, 2H), 7.31 (br d, J = 7.2 Hz, 1H), 6.98 (br t, J = 8.3 Hz, |
| | | | 1H), 6.55-6.42 (m, 3H), 6.36 (br t, J = 6.7 Hz, 1H), 6.07 (br d, |
| | | | J = 7.7 Hz, 1H), 4.44-4.36 (m, 1H), 4.35-4.29 (m, 1H), 3.96 (br |
| | | | d, J = 11.9 Hz, 1H), 3.71-3.60 (m, 1H), 3.34 (br s, 4H), 3.07- |
| | | | 2.96 (m, 1H), 2.79-2.70 (m, 2H), 2.60 (br d, J = 2.6 Hz, 2H), |
| | | | 2.56 (br s, 2H), 2.12-2.04 (m, 1H), 2.02-1.96 (m, 2H), 1.94- |
| | | | 1.85 (m, 2H), 1.80 (br d, J = 3.3 Hz, 1H), 1.79-1.70 (m, 6H), |
| | | | 1.69 (br s, 1H), 1.52-1.42 (m, 2H), 1.40-1.21 (m, 3H), 1.12- |
| | | | 0.91 (m, 2H) |
| 457 | 792 | 793 | δ (DMSO-d6) 10.79 (s, 1H), 8.47 (d, J = 3.6 Hz, 1H), 8.05 (br d, |
| | | | J = 8.0 Hz, 1H), 7.98 (s, 1H), 7.75-7.68 (m, 2H), 7.62-7.59 (m, |
| | | | 1H), 7.58-7.48 (m, 2H), 7.23 (br d, J = 8.6 Hz, 1H), 7.03-6.98 |
| | | | (m, 1H), 6.55-6.50 (m, 1H), 6.47-6.41 (m, 2H), 6.39-6.33 (m, |
| | | | 1H), 6.02-5.96 (m, 1H), 4.35-4.27 (m, 1H), 3.70-3.58 (m, 1H), |
| | | | 3.57-3.40 (m, 2H), 3.04-2.97 (m, 2H), 2.60 (br d, J = 3.5 Hz, |
| | | | 1H), 2.33-2.15 (m, 3H), 2.13-2.05 (m, 2H), 2.05-1.93 (m, 4H), |
| | | | 1.89-1.84 (m, 2H), 1.83-1.75 (m, 4H), 1.68-1.63 (m, 2H), 1.62- |
| | | | 1.53 (m, 2H), 1.48-1.41 (m, 2H), 1.40-1.35 (m, 2H), 1.34 (br |
| | | | d, J = 1.2 Hz, 3H), 1.26-1.23 (m, 1H) |
| 458 | 797 | 798 | δ (DMSO-d6) 10.78 (br s, 1H), 8.43 (d, J = 3.6 Hz, 1H), 7.88- |
| | | | 7.82 (m, 2H), 7.62-7.57 (m, 1H), 7.56-7.53 (m, 1H), 7.46 (br |
| | | | d, J = 7.9 Hz, 1H), 7.19 (br d, J = 7.6 Hz, 1H), 6.82 (t, J = 9.4 Hz, |
| | | | 1H), 6.51 (dd, J = 15.1, 2.2 Hz, 1H), 6.42 (br d, J = 8.7 Hz, 1H), |
| | | | 5.80 (br d, J = 7.7 Hz, 1H), 4.30-4.22 (m, 1H), 3.65 (br t, J = 5.2 |
| | | | Hz, 3H), 3.54-3.45 (m, 2H), 2.83 (br s, 3H), 2.75-2.68 (m, 2H), |
| | | | 2.60 (br d, J = 4.5 Hz, 4H), 2.43 (br t, J = 6.3 Hz, 2H), 2.30-2.21 |
| | | | (m, 1H), 2.09 (td, J = 8.6, 4.1 Hz, 1H), 1.98 (br d, J = 11.8 Hz, |
| | | | 2H), 1.91-1.84 (m, 7H), 1.82-1.74 (m, 4H), 1.43-1.30 (m, 5H), |
| | | | 1.30-1.18 (m, 4H) |
| 459 | 821 | 822 | δ 10.77 (s, 1H), 8.45 (br d, J = 3.4 Hz, 1H), 8.06 (br d, J = 7.6 Hz, |
| | | | 1H), 7.97 (br s, 1H), 7.75-7.64 (m, 3H), 7.59 (br d, J = 8.3 Hz, |
| | | | 1H), 7.57-7.51 (m, 1H), 7.21 (br d, J = 7.8 Hz, 1H), 6.85-6.78 |
| | | | (m, 1H), 6.54-6.47 (m, 2H), 6.41 (br d, J = 8.6 Hz, 1H), 6.35 (br |
| | | | t, J = 6.6 Hz, 1H), 5.79 (br d, J = 7.6 Hz, 1H), 4.30-4.20 (m, 1H), |
| | | | 3.71-3.57 (m, 1H), 2.95-2.78 (m, 6H), 2.73 (ddd, J = 17.7, 12.4, |
| | | | 5.3 Hz, 1H), 2.43 (br s, 3H), 2.16-2.05 (m, 4H), 1.98 (br d, J = |
| | | | 5.6 Hz, 2H), 1.91-1.64 (m, 8H), 1.50-1.37 (m, 3H), 1.37-1.22 |
| | | | (m, 3H), 1.07-0.73 (m, 5H) |
| 460 | 821 | 822 | δ 10.82-10.66 (m, 1H), 8.46 (br s, 1H), 8.12-8.02 (m, 1H), 7.97 |
| | | | (br s, 1H), 7.75-7.64 (m, 3H), 7.62-7.50 (m, 2H), 7.21 (br d, |
| | | | J = 7.5 Hz, 1H), 6.82 (br t, J = 9.3 Hz, 1H), 6.56-6.46 (m, 2H), |
| | | | 6.41 (br d, J = 7.6 Hz, 1H), 6.35 (br t, J = 6.4 Hz, 1H), 5.79 (br d, |
| | | | J = 7.3 Hz, 1H), 4.32-4.19 (m, 1H), 3.70-3.58 (m, 1H), 2.91- |
| | | | 2.80 (m, 6H), 2.75-2.65 (m, 1H), 2.43 (br s, 3H), 2.11 (br d, J = |
| | | | 6.1 Hz, 4H), 1.98 (br d, J = 9.1 Hz, 2H), 1.89-1.64 (m, 8H), 1.44 |
| | | | (br d, J = 11.3 Hz, 3H), 1.33-1.19 (m, 3H), 0.98-0.60 (m, 5H) |
| 461 | 799 | 800 | δ (DMSO-d6) 10.78 (br s, 1H), 8.44 (br s, 1H), 7.96 (br s, 1H), |
| | | | 7.84 (br s, 1H), 7.63-7.52 (m, 3H), 7.19 (br s, 1H), 6.86-6.78 |
| | | | (m, 1H), 6.55-6.48 (m, 1H), 6.43 (br d, J = 5.1 Hz, 1H), 5.80 |
| | | | (br d, J = 6.3 Hz, 1H), 4.38 (br d, J = 4.4 Hz, 2H), 4.30-4.23 |
| | | | (m, 1H), 3.72 (br d, J = 5.1 Hz, 3H), 2.84 (br s, 4H), 2.61 (br d, |
| | | | J = 0.9 Hz, 6H), 2.29-2.21 (m, 1H), 2.17-2.08 (m, 3H), 1.97 |
| | | | (br s, 3H), 1.91-1.84 (m, 3H), 1.78 (br s, 4H), 1.45-1.31 (m, |
| | | | 5H), 1.30-1.19 (m, 4H) |
| 462 | 799 | 800 | δ (DMSO-d6) 10.78 (s, 1H), 8.44 (d, J = 3.7 Hz, 1H), 7.96 (s, |
| | | | 1H), 7.84 (br d, J = 5.8 Hz, 1H), 7.64-7.58 (m, 1H), 7.57-7.52 |
| | | | (m, 2H), 7.19 (br d, J = 7.7 Hz, 1H), 6.82 (t, J = 9.4 Hz, 1H), 6.52 |
| | | | (dd, J = 15.1, 2.3 Hz, 1H), 6.43 (dd, J = 8.8, 1.8 Hz, 1H), 5.82 (br |
| | | | d, J = 7.6 Hz, 1H), 4.38 (t, J = 5.4 Hz, 2H), 4.30-4.23 (m, 1H), |
| | | | 3.72 (t, J = 6.0 Hz, 2H), 3.68-3.61 (m, 1H), 3.50 (br d, J = 7.2 |
| | | | Hz, 1H), 2.87 (br s, 3H), 2.79-2.73 (m, 1H), 2.73-2.66 (m, 4H), |
| | | | 2.60 (br t, J = 4.1 Hz, 1H), 2.39-2.32 (m, 1H), 2.17-2.07 (m, |
| | | | 3H), 2.02-1.94 (m, 3H), 1.93-1.84 (m, 3H), 1.82-1.74 (m, 4H), |
| | | | 1.42-1.32 (m, 4H), 1.31-1.20 (m, 5H) |
| 463 | 792 | 793 | δ 10.78 (s, 1H), 8.45 (d, J = 3.7 Hz, 1H), 8.27 (br s, 1H), 7.97 (br |
| | | | d, J = 6.4 Hz, 1H), 7.85 (br d, J = 8.0 Hz, 1H), 7.76-7.69 (m, |
| | | | 2H), 7.67-7.61 (m, 1H), 7.52 (t, J = 7.6 Hz, 2H), 7.46-7.37 (m, |
| | | | 1H), 7.27 (br d, J = 7.5 Hz, 1H), 6.93-6.77 (m, 1H), 6.61-6.49 |
| | | | (m, 1H), 6.43 (br d, J = 8.5 Hz, 1H), 6.02-5.72 (m, 1H), 4.26 (br |
| | | | d, J = 4.6 Hz, 1H), 4.14-3.95 (m, 1H), 3.79-3.56 (m, 3H), 3.25- |
| | | | 3.11 (m, 3H), 3.09-2.95 (m, 2H), 2.92-2.79 (m, 2H), 2.77- |
| | | | 2.65 (m, 3H), 2.64-2.57 (m, 3H), 2.41-2.25 (m, 1H), 2.18-1.98 |
| | | | (m, 3H), 1.95-1.79 (m, 1H), 1.73 (br d, J = 12.3 Hz, 3H), 1.62- |
| | | | 1.26 (m, 8H) |
| 464 | 791 | 792 | δ 10.80 (s, 1H), 8.45 (d, J = 3.4 Hz, 1H), 8.26 (br s, 1H), 8.13 (s, |
| | | | 0.2H), 8.00-7.91 (m, 1H), 7.84 (br d, J = 7.6 Hz, 1H), 7.71 (br |
| | | | d, J = 7.5 Hz, 2H), 7.68-7.61 (m, 1H), 7.51 (t, J = 7.5 Hz, 2H), |
| | | | 7.44-7.38 (m, 1H), 7.28 (br d, J = 7.5 Hz, 1H), 7.03-6.93 (m, |
| | | | 1H), 6.50-6.41 (m, 2H), 6.06 (br d, J = 7.1 Hz, 1H), 4.38-4.25 |
| | | | (m, 1H), 4.15-4.03 (m, 1H), 3.83-3.59 (m, 3H), 3.05-2.87 (m, |
| | | | 2H), 2.87-2.64 (m, 4H), 2.63-2.53 (m, 3H), 2.16-1.71 (m, |
| | | | 12H), 1.64-1.19 (m, 9H) |
| 465 | 793 | 794 | δ 10.78 (s, 1H), 8.47 (d, J = 3.6 Hz, 1H), 8.05 (br d, J = 7.9 Hz, |
| | | | 1H), 7.97 (s, 1H), 7.75-7.66 (m, 2H), 7.62-7.51 (m, 2H), 7.35 |
| | | | (br d, J = 7.8 Hz, 1H), 6.98 (t, J = 8.8 Hz, 1H), 6.51 (d, J = 9.3 |
| | | | Hz, 1H), 6.48-6.39 (m, 2H), 6.35 (dt, J = 1.3, 6.7 Hz, 1H), 5.99 |
| | | | (d, J = 7.9 Hz, 1H), 4.37-4.19 (m, 1H), 3.98-3.76 (m, 1H), 3.54 |
| | | | (br d, J = 12.5 Hz, 5H), 2.93-2.79 (m, 4H), 2.74-2.66 (m, 3H), |
| | | | 2.18-2.04 (m, 3H), 2.01-1.79 (m, 6H), 1.72-1.58 (m, 8H), 1.49- |
| | | | 1.36 (m, 2H), 1.10-0.96 (m, 2H) |
| 466 | 775 | 776 | δ 10.78 (s, 1H), 8.45 (d, J = 3.7 Hz, 1H), 8.26 (br s, 1H), 8.14 (s, |
| | | | 1H), 7.96 (br d, J = 6.1 Hz, 1H), 7.84 (0.3r d, J = 8.0 Hz, 1H), |
| | | | 7.75-7.68 (m, 2H), 7.67-7.62 (m, 1H), 7.54-7.48 (m, 2H), 7.45- |
| | | | 7.36 (m, 1H), 7.25 (d, J = 7.6 Hz, 1H), 7.02-6.94 (m, 1H), 6.49- |
| | | | 6.38 (m, 2H), 6.01 (br d, J = 7.6 Hz, 1H), 4.39 (br d, J = 12.5 |
| | | | Hz, 1H), 4.34-4.25 (m, 1H), 3.94 (br d, J = 11.7 Hz, 1H), 3.72- |
| | | | 3.60 (m, 1H), 3.10-2.95 (m, 3H), 2.80-2.67 (m, 1H), 2.65-2.53 |
| | | | (m, 3H), 2.37-2.24 (m, 2H), 2.22-1.97 (m, 5H), 1.92-1.59 (m, |
| | | | 10H), 1.54-1.19 (m, 5H), 1.08-0.81 (m, 2H) |
| 467 | 829 | 830 | δ (DMSO-d6) 10.87 (s, 1H), 8.47 (d, J = 3.6 Hz, 1H), 8.18 (s, |
| | | | 1H), 8.06 (br d, J = 7.9 Hz, 1H), 7.98 (s, 1H), 7.75-7.66 (m, 2H), |
| | | | 7.60 (d, J = 8.5 Hz, 2H), 7.54 (ddd, J = 9.1, 6.8, 2.0 Hz, 1H), 7.44 |
| | | | (s, 1H), 7.25 (d, J = 7.8 Hz, 1H), 7.03 (d, J = 8.3 Hz, 1H), 6.51 |
| | | | (d, J = 9.1 Hz, 1H), 6.35 (dt, J = 6.7, 1.3 Hz, 1H), 4.49 (br s, 1H), |
| | | | 4.32 (dd, J = 9.7, 5.1 Hz, 1H), 3.97 (s, 3H), 3.73 (br s, 1H), 2.96 |
| | | | (br d, J = 10.8 Hz, 2H), 2.83-2.70 (m, 2H), 2.68-2.59 (m, 3H), |
| | | | 2.34 (dtd, J = 4.8, 9.4, 13.9 Hz, 1H), 2.20-2.13 (m, 3H), 2.06- |
| | | | 1.88 (m, 7H), 1.83-1.67 (m, 8H), 1.46-1.37 (m, 4H), 1.03-0.92 |
| | | | (m, 2H) |
| 468 | 812 | 813 | δ (DMSO-d6) 10.78 (s, 1H), 8.44 (d, J = 3.5 Hz, 1H), 8.30 (s, |
| | | | 1H), 8.16-7.99 (m, 2H), 7.72-7.65 (m, 1H), 7.65-7.58 (m, 1H), |
| | | | 7.28 (br d, J = 7.6 Hz, 1H), 7.02-6.95 (m, 1H), 6.48-6.39 (m, |
| | | | 2H), 5.99 (br d, J = 7.9 Hz, 1H), 5.25-4.75 (m, 1H), 4.42-4.23 |
| | | | (m, 3H), 3.99-3.88 (m, 1H), 3.71-3.50 (m, 6H), 3.05-2.97 (m, |
| | | | 1H), 2.91 (br d, J = 10.6 Hz, 2H), 2.80-2.67 (m, 1H), 2.61-2.53 |
| | | | (m, 3H), 2.13 (br d, J = 6.4 Hz, 2H), 2.10-2.04 (m, 1H), 2.02- |
| | | | 1.90 (m, 5H), 1.88-1.84 (m, 1H), 1.78 (br d, J = 9.6 Hz, 2H), |
| | | | 1.70 (br d, J = 13.3 Hz, 4H), 1.62 (br s, 4H), 1.54-1.42 (m, 2H), |
| | | | 1.39-1.28 (m, 2H), 1.06-0.95 (m, 1H), 0.94-0.82 (m, 1H) |
| 469 | 805 | 806 | δ (DMSO-d6) 10.80 (s, 1H), 8.46 (d, J = 3.4 Hz, 1H), 8.26 (s, |
| | | | 1H), 8.16 (s, 1H), 8.00-7.94 (m, 1H), 7.85 (br d, J = 7.9 Hz, 1H), |
| | | | 7.72 (d, J = 7.6 Hz, 2H), 7.65 (t, J = 7.6 Hz, 1H), 7.55-7.49 (m, |
| | | | 2H), 7.46-7.40 (m, 1H), 7.26-7.21 (m, 1H), 7.02-6.95 (m, 1H), |
| | | | 6.50-6.42 (m, 2H), 6.06-6.01 (m, 1H), 4.56-4.45 (m, 1H), 4.36- |
| | | | 4.27 (m, 1H), 3.96 (br d, J = 12.1 Hz, 2H), 3.74 (br dd, J = 8.3, |
| | | | 13.4 Hz, 1H), 3.11 (br d, J = 9.9 Hz, 2H), 2.82-2.65 (m, 5H), |
| | | | 2.62-2.58 (m, 1H), 2.30-2.20 (m, 2H), 2.13-2.04 (m, 2H), 2.04- |
| | | | 1.94 (m, 4H), 1.74-1.64 (m, 6H), 1.53-1.38 (m, 8H), 1.08- |
| | | | 0.97 (m, 2H) |
| 470 | 823 | 824 | δ (DMSO-d6) 10.87-10.69 (m, 1H), 8.46 (d, J = 3.6 Hz, 1H), |
| | | | 8.05 (br d, J = 7.1 Hz, 1H), 7.96 (s, 1H), 7.68 (t, J = 7.9 Hz, 1H), |
| | | | 7.61 (d, J = 7.1 Hz, 1H), 7.57 (d, J = 8.2 Hz, 1H), 7.28 (d, J = 7.7 |
| | | | Hz, 1H), 6.82 (t, J = 9.3 Hz, 1H), 6.50 (dd, J = 15.1, 2.4 Hz, 1H), |
| | | | 6.42 (dd, J = 8.5, 1.9 Hz, 1H), 6.33 (s, 1H), 6.22 (dd, J = 7.1, 1.7 |
| | | | Hz, 1H), 5.79 (d, J = 7.4 Hz, 1H), 4.36-4.20 (m, 2H), 4.10-3.94 |
| | | | (m, 1H), 3.73-3.59 (m, 2H), 3.02-2.91 (m, 1H), 2.84 (br s, 4H), |
| | | | 2.77-2.70 (m, 1H), 2.69-2.61 (m, 4H), 2.60-2.53 (m, 2H), 2.31 |
| | | | (s, 2H), 2.21 (s, 3H), 2.09 (td, J = 8.8, 4.2 Hz, 1H), 1.99 (br d, |
| | | | J = 11.6 Hz, 2H), 1.91-1.77 (m, 1H), 1.70 (br d, J = 12.7 Hz, 2H), |
| | | | 1.61-1.24 (m, 9H) |
| 471 | 807 | 808 | δ (DMSO-d6) 10.78 (s, 1H), 8.47 (d, J = 3.7 Hz, 1H), 8.14 (s, |
| | | | 1H), 8.05 (br d, J = 7.2 Hz, 1H), 7.96 (s, 1H), 7.69 (t, J = 7.9 Hz, |
| | | | 1H), 7.63-7.55 (m, 2H), 7.28 (br d, J = 7.6 Hz, 1H), 6.88-6.81 |
| | | | (m, 1H), 6.46-6.44 (m, 1H), 6.54-6.42 (m, 2H), 6.33 (s, 1H), |
| | | | 6.23 (dd, J = 1.7, 7.1 Hz, 1H), 5.86-5.80 (m, 1H), 4.44-4.35 |
| | | | (m, 1H), 4.30-4.23 (m, 1H), 3.99-3.91 (m, 1H), 3.70-3.59 (m, |
| | | | 2H), 3.07-2.96 (m, 2H), 2.95-2.85 (m, 4H), 2.78-2.71 (m, 2H), |
| | | | 2.59 (br d, J = 3.8 Hz, 3H), 2.21 (s, 3H), 2.17-2.06 (m, 2H), 2.03- |
| | | | 1.96 (m, 2H), 1.92-1.76 (m, 4H), 1.76-1.68 (m, 3H), 1.52- |
| | | | 1.43 (m, 2H), 1.41-1.23 (m, 3H), 1.09-1.00 (m, 1H), 0.97-0.86 |
| | | | (m, 1H) |
| 472 | 822 | 823 | δ (DMSO-d6) 10.79 (s, 1H), 8.49-8.44 (m, 1H), 8.15 (s, 1H), |
| | | | 8.05 (br d, J = 6.8 Hz, 1H), 7.99-7.93 (m, 1H), 7.72-7.65 (m, |
| | | | 1H), 7.59 (br dd, J = 7.5, 17.6 Hz, 2H), 7.29 (br d, J = 7.5 Hz, |
| | | | 1H), 7.02-6.94 (m, 1H), 6.48-6.40 (m, 2H), 6.33 (s, 1H), 6.22 |
| | | | (br d, J = 7.2 Hz, 1H), 6.01 (br d, J = 7.5 Hz, 1H), 4.35-4.25 (m, |
| | | | 1H), 4.10-4.02 (m, 1H), 3.71-3.61 (m, 2H), 3.06 (br d, J = 10.0 |
| | | | Hz, 2H), 2.98-2.91 (m, 1H), 2.79-2.68 (m, 2H), 2.59 (br d, J = |
| | | | 3.9 Hz, 3H), 2.35 (br s, 3H), 2.32-2.25 (m, 2H), 2.21 (s, 3H), |
| | | | 2.12-2.04 (m, 2H), 2.00 (br d, J = 9.9 Hz, 2H), 1.93-1.81 (m, |
| | | | 2H), 1.69 (br s, 3H), 1.63-1.57 (m, 2H), 1.54 (br s, 1H), 1.44 (br |
| | | | d, J = 9.4 Hz, 2H), 1.41-1.30 (m, 3H) |
| 473 | 806 | 807 | δ (DMSO-d6) 10.79 (s, 1H), 8.47 (d, J = 3.5 Hz, 1H), 8.05 (br d, |
| | | | J = 7.6 Hz, 1H), 7.96 (s, 1H), 7.72-7.66 (m, 1H), 7.61 (d, J = 6.9 |
| | | | Hz, 1H), 7.59-7.56 (m, 1H), 7.28 (br d, J = 7.5 Hz, 1H), 7.02- |
| | | | 6.96 (m, 1H), 6.48-6.42 (m, 2H), 6.33 (s, 1H), 6.22 (dd, J = 7.1, |
| | | | 1.7 Hz, 1H), 6.00 (d, J = 7.6 Hz, 1H), 4.39 (br d, J = 11.6 Hz, 1H), |
| | | | 4.31 (ddd, J = 11.8, 7.4, 4.9 Hz, 1H), 3.97-3.90 (m, 1H), 3.68- |
| | | | 3.60 (m, 1H), 3.04-2.96 (m, 1H), 2.92 (br d, J = 10.7 Hz, 2H), |
| | | | 2.79-2.67 (m, 1H), 2.59 (br d, J = 4.2 Hz, 1H), 2.56 (br d, J = |
| | | | 3.8 Hz, 1H), 2.21 (s, 4H), 2.14 (br d, J = 6.2 Hz, 1H), 2.12-2.06 |
| | | | (m, 1H), 2.02-1.91 (m, 5H), 1.87 (br dd, J = 12.3, 4.4 Hz, 1H), |
| | | | 1.78 (br d, J = 8.7 Hz, 2H), 1.70 (br d, J = 11.4 Hz, 3H), 1.63 (br |
| | | | s, 4H), 1.51-1.41 (m, 3H), 1.37-1.31 (m, 2H), 1.07-0.98 (m, |
| | | | 1H), 0.88 (br d, J = 12.3 Hz, 1H) |
| 474 | 794 | 495 | δ 10.78 (s, 1H), 8.47 (d, J = 3.6 Hz, 1H), 8.18 (s, 0.2H), 8.06 (br |
| | | | d, J = 7.6 Hz, 1H), 7.97 (s, 1H), 7.74-7.67 (m, 2H), 7.62-7.57 |
| | | | (m, 1H), 7.54 (ddd, J = 9.2, 6.8,2.0 Hz, 1H), 7.25 (d, J = 7.6 Hz, |
| | | | 1H), 7.03-6.92 (m, 1H), 6.51 (d, J = 9.4 Hz, 1H), 6.47-6.39 (m, |
| | | | 2H), 6.35 (dt, J = 6.8, 1.2 Hz, 1H), 5.99 (d, J = 7.8 Hz, 1H), 4.48 |
| | | | (br s, 1H), 4.36-4.24 (m, 1H), 4.00 (br d, J = 12.4 Hz, 2H), 3.80- |
| | | | 3.66 (m, 1H), 2.92 (br d, J = 10.8 Hz, 2H), 2.83-2.65 (m, 3H), |
| | | | 2.61-2.52 (m, 2H), 2.47-2.41 (m, 1H), 2.24 (br t, J = 10.4 Hz, |
| | | | 2H), 2.13-2.03 (m, 1H), 2.02-1.92 (m, 4H), 1.91-1.80 (m, 1H), |
| | | | 1.74 (br d, J = 11.0 Hz, 2H), 1.68-1.53 (m, 4H), 1.48-1.26 (m, |
| | | | 6H) |
| 475 | 795 | 796 | δ 10.80 (s, 1H), 8.50 (d, J = 3.8 Hz, 1H), 8.17 (s, 0.18H), 8.09 (br |
| | | | d, J = 7.2 Hz, 1H), 8.00 (s, 1H), 7.76-7.69 (m, 2H), 7.62 (br d, |
| | | | J = 8.0 Hz, 1H), 7.60-7.54 (m, 1H), 7.28 (d, J = 7.8 Hz, 1H), 6.88- |
| | | | 6.81 (m, 1H), 6.57-6.50 (m, 2H), 6.47-6.42 (m, 1H), 6.38 (t, |
| | | | J = 6.6 Hz, 1H), 5.83 (d, J-7.8 Hz, 1H), 4.56-4.47 (m, 1H), |
| | | | 4.32-4.24 (m, 1H), 4.07-3.97 (m, 2H), 3.81-3.72 (m, 1H), 2.87 |
| | | | (br s, 4H), 2.81-2.70 (m, 3H), 2.64 (br s, 5H), 2.47-2.39 (m, |
| | | | 1H), 2.16-2.07 (m, 1H), 1.99 (br dd, J = 5.6, 3.4 Hz, 4H), 1.87 |
| | | | (br dd, J = 12.2, 4.6 Hz, 1H), 1.83-1.77 (m, 2H), 1.48-1.39 (m, |
| | | | 4H), 1.38-1.27 (m, 2H) |
| 476 | 817 | 818 | δ (DMSO-d6) 10.78 (s, 1H), 8.44 (d, J = 3.5 Hz, 1H), 8.15 (s, |
| | | | 1H), 8.00 (s, 1H), 7.88 (br d, J = 6.4 Hz, 1H), 7.68-7.62 (m, 1H), |
| | | | 7.62-7.56 (m, 2H), 7.20 (br d, J = 7.8 Hz, 1H), 6.83 (t, J = 9.4 |
| | | | Hz, 1H), 6.55-6.40 (m, 2H), 5.83 (br d, J = 7.9 Hz, 1H), 5.13- |
| | | | 4.91 (m, 1H), 4.25 (s, 2H), 4.03-3.99 (m, 2H), 3.82-3.78 (m, |
| | | | 2H), 3.71-3.62 (m, 1H), 3.59-3.50 (m, 1H), 3.26-3.20 (m, 1H), |
| | | | 3.07-2.95 (m, 2H), 2.86-2.64 (m, 5H), 2.61-2.55 (m, 2H), 2.14- |
| | | | 2.04 (m, 3H), 2.03-1.95 (m, 3H), 1.91-1.83 (m, 1H), 1.75 (br |
| | | | d, J = 9.8 Hz, 5H), 1.52-1.38 (m, 4H), 1.36-1.21 (m, 3H) |
| 477 | 782 | 783 | δ 10.78 (s, 1H), 8.37 (d, J = 3.9 Hz, 1H), 7.55 (br d, J = 6.8 Hz, |
| | | | 1H), 7.34 (br d, J = 4.5 Hz, 2H), 7.16 (d, J = 7.5 Hz, 1H), 7.10 |
| | | | (dt, J = 2.7, 4.5 Hz, 1H), 7.03-6.94 (m, 1H), 6.57-6.35 (m, 2H), |
| | | | 5.99 (d, J = 7.6 Hz, 1H), 4.42-4.25 (m, 2H), 3.99-3.88 (m, 1H), |
| | | | 3.74-3.53 (m, 2H), 3.24-3.17 (m, 4H), 3.04-2.88 (m, 3H), 2.79- |
| | | | 2.65 (m, 2H), 2.60-2.55 (m, 2H), 2.15-1.98 (m, 5H), 1.97- |
| | | | 1.84 (m, 3H), 1.79-1.67 (m, 5H), 1.63 (br d, J = 4.6 Hz, 7H), |
| | | | 1.56 (br d, J = 4.9 Hz, 2H), 1.52-1.42 (m, 2H), 1.41-1.19 (m, |
| | | | 3H), 1.07-0.82 (m, 2H) |
| 478 | 754 | 755 | δ 10.77 (s, 1H), 8.43-8.35 (m, 1H), 7.74-7.65 (m, 2H), 7.40 (t, |
| | | | J = 7.7 Hz, 1H), 7.26-7.12 (m, 2H), 6.85-6.78 (m, 1H), 6.51 |
| | | | (dd, J = 2.2, 15.1 Hz, 1H), 6.42 (dd, J = 2.1, 8.8 Hz, 1H), 5.82 (br |
| | | | d, J = 7.5 Hz, 1H), 4.32-4.21 (m, 2H), 3.74-3.61 (m, 1H), 2.89- |
| | | | 2.89 (m, 3H), 2.87 (s, 3H), 2.80-2.66 (m, 6H), 2.57 (br d, J = |
| | | | 17.5 Hz, 2H), 2.15-1.95 (m, 4H), 1.93-1.81 (m, 3H), 1.79-1.69 |
| | | | (m, 3H), 1.66-1.54 (m, 2H), 1.52-1.32 (m, 8H), 1.03-0.96 (m, |
| | | | 2H), 0.74-0.66 (m, 2H) |
| 479 | 770 | 771 | δ 10.78 (s, 1H), 8.18-8.17 (m, 1H), 8.12 (d, J = 6.0 Hz, 1H), 7.56 |
| | | | (br d, J = 7.8 Hz, 1H), 7.19-7.13 (m, 2H), 7.01-6.92 (m, 2H), |
| | | | 6.47-6.40 (m, 2H), 6.01-5.97 (m, 1H), 4.85 (br d, J = 8.0 Hz, |
| | | | 2H), 4.34-4.25 (m, 1H), 3.57-3.49 (m, 2H), 2.99-2.90 (m, 2H), |
| | | | 2.73 (s, 1H), 2.64-2.54 (m, 2H), 2.40-2.25 (m, 5H), 2.06 (br s, |
| | | | 2H), 1.96-1.84 (m, 7H), 1.80 (br d, J = 10.8 Hz, 3H), 1.73-1.56 |
| | | | (m, 6H), 1.47-1.37 (m, 2H), 1.32 (br d, J = 11.0 Hz, 2H), 1.25- |
| | | | 1.10 (m, 4H) |
| 480 | 799 | 800 | δ 10.83-10.70 (m, 1H), 8.64 (d, J = 5.1 Hz, 2H), 8.51 (d, J = 3.3 |
| | | | Hz, 1H), 7.73 (br d, J = 2.4 Hz, 1H), 7.56 (d, J = 7.8 Hz, 1H), |
| | | | 7.32 (d, J = 7.8 Hz, 1H), 7.01-6.92 (m, 1H), 6.46-6.38 (m, 2H), |
| | | | 5.98 (d, J = 7.9 Hz, 1H), 4.29 (s, 2H), 4.01 (s, 4H), 3.69-3.62 |
| | | | (m, 1H), 2.88 (br d, J = 10.6 Hz, 2H), 2.78-2.66 (m, 1H), 2.60- |
| | | | 2.53 (m, 1H), 2.33-2.19 (m, 4H), 2.13-1.92 (m, 5H), 1.90-1.68 |
| | | | (m, 8H), 1.67-1.53 (m, 4H), 1.45 (br d, J = 12.5 Hz, 2H), 1.28 |
| | | | (br t, J = 11.9 Hz, 4H), 1.20-1.09 (m, 2H) |
| 481 | 828 | 829 | δ (DMSO-d6) 10.86-10.73 (m, 1H), 8.45 (d, J = 3.5 Hz, 1H), |
| | | | 8.13 (br s, 1H), 8.06 (br d, J = 6.6 Hz, 1H), 7.72-7.66 (m, 1H), |
| | | | 7.65-7.59 (m, 1H), 7.31-7.25 (m, 1H), 6.98 (t, J = 8.8 Hz, 1H), |
| | | | 6.47-6.44 (m, 1H), 6.41 (d, J = 1.9 Hz, 1H), 5.98 (d, J = 7.6 Hz, |
| | | | 1H), 5.17-4.87 (m, 1H), 4.39-4.17 (m, 3H), 4.08-4.00 (m, 1H), |
| | | | 3.74-3.50 (m, 5H), 3.41 (br d, J = 13.1 Hz, 1H), 3.26-3.18 (m, |
| | | | 1H), 3.04-2.90 (m, 3H), 2.78-2.68 (m, 1H), 2.65-2.54 (m, 3H), |
| | | | 2.28 (s, 2H), 2.22 (br t, J = 10.8 Hz, 2H), 2.12-2.05 (m, 1H), |
| | | | 2.05-1.91 (m, 3H), 1.86 (dt, J = 12.1, 8.0 Hz, 2H), 1.70 (br t, |
| | | | J = 12.0 Hz, 3H), 1.64-1.55 (m, 3H), 1.52 (br d, J = 5.4 Hz, 2H), |
| | | | 1.49-1.24 (m, 6H) |
| 482 | 829 | 830 | δ (DMSO-d6) 10.86-10.66 (m, 1H), 8.44 (d, J = 3.5 Hz, 1H), |
| | | | 8.14-8.02 (m, 2H), 7.71-7.57 (m, 2H), 7.27 (br d, J = 7.3 Hz, |
| | | | 1H), 6.81 (t, J = 9.3 Hz, 1H), 6.53-6.38 (m, 2H), 5.78 (d, J = 7.6 |
| | | | Hz, 1H), 5.07-4.89 (m, 1H), 4.29-4.21 (m, 2H), 4.02 (br dd, |
| | | | J = 10.9, 1.8 Hz, 1H), 3.68-3.54 (m, 4H), 3.48-3.40 (m, 1H), 3.24- |
| | | | 3.16 (m, 1H), 2.97 (br s, 1H), 2.87-2.79 (m, 4H), 2.69 (br s, |
| | | | 1H), 2.68-2.61 (m, 4H), 2.58 (br d, J = 4.0 Hz, 1H), 2.34-2.29 |
| | | | (m, 2H), 2.11-2.04 (m, 1H), 2.03-1.93 (m, 3H), 1.92-1.76 (m, |
| | | | 3H), 1.71 (br d, J = 11.8 Hz, 2H), 1.62-1.43 (m, 5H), 1.42-1.26 |
| | | | (m, 4H), 1.23 (s, 1H) |
| 483 | 813 | 814 | δ (DMSO-d6) 10.97-10.41 (m, 1H), 8.44 (d, J = 3.5 Hz, 1H), |
| | | | 8.16-8.01 (m, 2H), 7.72-7.56 (m, 2H), 7.27 (br d, J = 7.5 Hz, |
| | | | 1H), 6.82 (t, J = 9.3 Hz, 1H), 6.50 (dd, J =15.0, 2.3 Hz, 1H), 6.42 |
| | | | (dd, J = 8.8, 2.0 Hz, 1H), 5.79 (d, J = 7.5 Hz, 1H), 5.08-4.91 (m, |
| | | | 1H), 4.40-4.32 (m, 1H), 4.27-4.22 (m, 1H), 3.94 (br d, J = 13.1 |
| | | | Hz, 1H), 3.70-3.50 (m, 4H), 3.40 (br d, J = 13.0 Hz, 1H), 3.04- |
| | | | 2.95 (m, 1H), 2.88-2.79 (m, 4H), 2.78-2.66 (m, 1H), 2.58 (br |
| | | | d, J = 4.1 Hz, 1H), 2.56-2.53 (m, 1H), 2.47 (br s, 3H), 2.17 (br |
| | | | d, J = 6.6 Hz, 2H), 2.12-1.91 (m, 5H), 1.91-1.66 (m, 8H), 1.47 |
| | | | (br d, J = 9.8 Hz, 2H), 1.42-1.22 (m, 3H), 1.09-0.96 (m, 1H), |
| | | | 0.89 (br d, J = 10.0 Hz, 1H) |
| 484 | 794 | 795 | δ (DMSO-d6) 10.78 (br s, 1H), 8.46 (d, J = 3.6 Hz, 1H), 8.27 (br |
| | | | s, 1H), 7.97 (br d, J = 5.6 Hz, 1H), 7.85 (br d, J = 7.5 Hz, 1H), |
| | | | 7.75-7.62 (m, 3H), 7.52 (t, J = 7.6 Hz, 2H), 7.46-7.39 (m, 1H), |
| | | | 7.28 (br d, J = 7.5 Hz, 1H), 6.83 (br t, J = 9.4 Hz, 1H), 6.51 (br d, |
| | | | J = 15.0 Hz, 1H), 6.42 (br d, J = 8.6 Hz, 1H), 5.81 (br d, J = 7.5 |
| | | | Hz, 1H), 4.26 (br t, J = 11.7 Hz, 1H), 4.13 (br d, J = 12.4 Hz, 1H), |
| | | | 3.79 (br d, J = 12.9 Hz, 1H), 3.67 (br s, 1H), 2.96-2.88 (m, 1H), |
| | | | 2.85 (br s, 4H), 2.79-2.67 (m, 2H), 2.61 (br d, J = 7.6 Hz, 6H), |
| | | | 2.55 (br s, 1H), 2.16-1.99 (m, 3H), 1.96-1.77 (m, 4H), 1.73 (br |
| | | | s, 2H), 1.60 (br d, J = 14.9 Hz, 1H), 1.48 (br s, 2H), 1.45-1.31 |
| | | | (m, 3H) |
| 485 | 776 | 777 | δ (DMSO-d6) 10.77 (s, 1H), 8.45 (d, J = 3.8 Hz, 1H), 8.27 (br s, |
| | | | 1H), 7.97 (br d, J = 7.1 Hz, 1H), 7.85 (br d, J = 7.8 Hz, 1H), 7.74- |
| | | | 7.70 (m, 2H), 7.67-7.63 (m, 1H), 7.52 (t, J = 7.6 Hz, 2H), 7.44- |
| | | | 7.40 (m, 1H), 7.25 (d, J = 7.5 Hz, 1H), 6.83 (t, J = 9.3 Hz, 1H), |
| | | | 6.51 (dd, J = 15.1, 2.4 Hz, 1H), 6.42 (dd, J = 8.8, 2.1 Hz, 1H), |
| | | | 5.80 (d, J = 7.8 Hz, 1H), 4.39 (br d, J = 12.4 Hz, 1H), 4.29-4.22 |
| | | | (m, 1H), 3.94 (br d, J = 12.8 Hz, 1H), 3.71-3.64 (m, 1H), 3.01 |
| | | | (br t, J = 12.3 Hz, 1H), 2.85 (br s, 4H), 2.78-2.67 (m, 1H), 2.59 |
| | | | (br d, J = 4.3 Hz, 1H), 2.57-2.53 (m, 2H), 2.48 (br s, 3H), 2.18 |
| | | | (br d, J = 6.6 Hz, 2H), 2.13-1.97 (m, 4H), 1.85 (br dd, J = 12.4, |
| | | | 4.3 Hz, 1H), 1.79 (br d, J = 8.9 Hz, 2H), 1.72 (br d, J = 12.0 Hz, |
| | | | 3H), 1.54-1.44 (m, 2H), 1.42-1.31 (m, 2H), 1.01 (br d, J = 11.6 |
| | | | Hz, 1H), 0.96-0.85 (m, 1H) |
| 486 | 817 | 818 | δ (DMSO-d6) 10.79 (s, 1H), 8.45 (d, J = 3.4 Hz, 1H), 8.22 (s, |
| | | | 1H), 7.97 (s, 1H), 7.87 (br d, J = 6.2 Hz, 1H), 7.68-7.49 (m, 4H), |
| | | | 7.21 (br d, J = 7.5 Hz, 1H), 6.84 (t, J = 9.3 Hz, 1H), 6.56-6.39 |
| | | | (m, 2H), 5.84 (d, J = 7.7 Hz, 1H), 5.10-4.91 (m, 1H), 4.39 (t, |
| | | | J = 5.3 Hz, 2H), 4.32-4.22 (m, 1H), 3.73 (t, J = 5.9 Hz, 2H), 3.69- |
| | | | 3.62 (m, 1H), 3.55-3.45 (m, 2H), 3.25-3.22 (m, 1H), 2.96 (br |
| | | | s, 2H), 2.82-2.70 (m, 2H), 2.38-2.28 (m, 3H), 2.18-2.09 (m, |
| | | | 4H), 2.03-1.94 (m, 3H), 1.79-1.68 (m, 6H), 1.51-1.28 (m, 7H) |
| 487 | 810 | 811 | δ (DMSO-d6) 10.79 (s, 1H), 8.47 (d, J = 3.6 Hz, 1H), 8.07 (br d, |
| | | | J = 7.3 Hz, 1H), 7.98 (s, 1H), 7.74-7.68 (m, 2H), 7.62-7.58 (m, |
| | | | 1H), 7.55 (ddd, J = 2.0, 6.8, 9.1 Hz, 1H), 7.30 (d, J = 7.6 Hz, 1H), |
| | | | 7.02-6.94 (m, 1H), 6.52 (d, J = 9.1 Hz, 1H), 6.44 (br d, J = 11.8 |
| | | | Hz, 2H), 6.36 (dt, J = 1.3, 6.8 Hz, 1H), 6.00 (br d, J = 7.1 Hz, |
| | | | 1H), 4.35-4.27 (m, 1H), 4.13 (dt, J = 3.9, 7.0 Hz, 1H), 3.85- |
| | | | 3.74 (m, 1H), 3.71-3.60 (m, 1H), 3.00-2.85 (m, 3H), 2.79-2.67 |
| | | | (m, 1H), 2.57 (br dd, J = 3.4, 13.4 Hz, 3H), 2.24-2.14 (m, 2H), |
| | | | 2.12-2.05 (m, 1H), 2.00 (br d, J = 9.6 Hz, 2H), 1.93-1.81 (m, |
| | | | 3H), 1.77-1.56 (m, 9H), 1.54-1.27 (m, 6H) |
| 488 | 807 | 808 | δ (DMSO-d6) 10.83-10.75 (m, 1H), 8.48 (br d, J = 2.1 Hz, 1H), |
| | | | 8.11-8.05 (m, 1H), 7.99 (br s, 1H), 7.75-7.68 (m, 2H), 7.63- |
| | | | 7.54 (m, 2H), 7.30 (br d, J = 4.9 Hz, 1H), 6.87-6.80 (m, 1H), |
| | | | 6.56-6.49 (m, 2H), 6.42 (br s, 1H), 6.39-6.34 (m, 1H), 5.81 (br |
| | | | d, J = 7.8 Hz, 1H), 4.31-4.20 (m, 2H), 3.71-3.61 (m, 2H), 2.86 |
| | | | (br s, 6H), 2.66-2.58 (m, 6H), 2.36-2.24 (m, 1H), 2.14-2.06 |
| | | | (m, 1H), 2.05-1.96 (m, 2H), 1.93-1.82 (m, 3H), 1.75-1.62 (m, |
| | | | 4H), 1.55-1.42 (m, 5H), 1.42-1.27 (m, 4H) |
| 489 | 828 | 829 | δ (DMSO-d6) 10.84 (br s, 1H), 8.45 (d, J = 3.6 Hz, 1H), 8.04 (d, |
| | | | J = 7.4 Hz, 1H), 7.96 (s, 1H), 7.74-7.66 (m, 2H), 7.61-7.47 (m, |
| | | | 4H), 7.21 (br d, J = 7.9 Hz, 1H), 6.91 (dd, J = 1.4, 9.1 Hz, 1H), |
| | | | 6.83 (s, 1H), 6.51 (d, J = 9.1 Hz, 1H), 6.35 (dt, J = 6.7, 1.3 Hz, |
| | | | 1H), 4.25 (dd, J = 9.1, 5.1 Hz, 1H), 3.88 (s, 3H), 3.69-3.58 (m, |
| | | | 1H), 3.53-3.43 (m, 1H), 3.20 (br s, 4H), 2.70-2.54 (m, 3H), |
| | | | 2.34-2.26 (m, 1H), 2.19-2.11 (m, 3H), 2.08-1.88 (m, 4H), 1.80 |
| | | | (br dd, J = 18.8, 12.8 Hz, 5H), 1.69 (br d, J = 10.9 Hz, 2H), 1.51 |
| | | | (br d, J = 3.4 Hz, 1H), 1.43-1.17 (m, 7H), 0.91-0.79 (m, 2H) |
| 490 | 799 | 800 | δ 10.79 (s, 1H), 10.28-10.12 (m, 1H), 8.44 (d, J = 3.6 Hz, 1H), |
| | | | 7.96 (s, 1H), 7.85 (br d, J = 6.0 Hz, 1H), 7.70 (br d, J = 7.8 Hz, |
| | | | 1H), 7.61-7.50 (m, 2H), 7.28-7.12 (m, 1H), 6.89 (t, J = 9.4 Hz, |
| | | | 1H), 6.56 (dd, J = 14.8, 2.0 Hz, 1H), 6.50-6.43 (m, 1H), 4.38 (t, |
| | | | J = 5.4 Hz, 2H), 4.29 (dd, J = 11.6, 5.0 Hz, 1H), 3.51 (br d, J = |
| | | | 9.0 Hz, 3H), 3.31-3.06 (m, 8H), 2.80-2.66 (m, 1H), 2.63-2.58 |
| | | | (m, 1H), 2.58-2.54 (m, 1H), 2.20-1.96 (m, 9H), 1.95-1.81 (m, |
| | | | 3H), 1.75 (br d, J = 12.0 Hz, 2H), 1.66-1.53 (m, 2H), 1.52-1.39 |
| | | | (m, 2H), 1.33-1.17 (m, 4H) |
| 491 | 801 | 802 | δ 10.77 (s, 1H), 8.38 (d, J = 3.8 Hz, 1H), 7.65-7.50 (m, 1H), 7.34 |
| | | | (br d, J = 4.8 Hz, 2H), 7.18 (br d, J = 7.3 Hz, 1H), 7.13-7.08 (m, |
| | | | 1H), 6.82 (br t, J = 8.8 Hz, 1H), 6.60-6.36 (m, 2H), 6.00-5.74 |
| | | | (m, 1H), 4.25 (br d, J = 7.5 Hz, 1H), 4.21-4.01 (m, 1H), 3.90- |
| | | | 3.72 (m, 1H), 3.70-3.55 (m, 2H), 3.22-3.16 (m, 4H), 2.96-2.79 |
| | | | (m, 5H), 2.76-2.65 (m, 3H), 2.59 (br d, J = 3.8 Hz, 5H), 2.15- |
| | | | 1.97 (m, 4H), 1.94 (br s, 3H), 1.76-1.68 (m, 3H), 1.63 (br d, J = |
| | | | 4.6 Hz, 5H), 1.56 (br d, J = 4.6 Hz, 2H), 1.50-1.42 (m, 2H), 1.39 |
| | | | (br s, 2H) |
| 492 | 756 | 757 | δ 10.77 (br d, J = 1.3 Hz, 1H), 8.39 (d, J = 3.7 Hz, 1H), 7.84- |
| | | | 7.58 (m, 2H), 7.40 (t, J = 7.8 Hz, 1H), 7.28-7.15 (m, 2H), 6.81 |
| | | | (t, J = 9.4 Hz, 1H), 6.57-6.34 (m, 2H), 5.79 (d, J = 7.6 Hz, 1H), |
| | | | 4.35-4.19 (m, 2H), 4.10-3.94 (m, 1H), 3.79-3.59 (m, 2H), 3.01- |
| | | | 2.93 (m, 1H), 2.83 (br s, 4H), 2.64 (br s, 5H), 2.60-2.54 (m, |
| | | | 2H), 2.30 (s, 2H), 2.08 (td, J = 4.2, 8.7 Hz, 1H), 2.04-1.96 (m, |
| | | | 3H), 1.84 (dq, J = 4.5, 12.2 Hz, 1H), 1.75-1.66 (m, 2H), 1.56- |
| | | | 1.26 (m, 9H), 1.04-0.96 (m, 2H), 0.74-0.68 (m, 2H) |
| 493 | 799 | 800 | δ 10.79 (s, 1H), 9.91 (br s, 1H), 8.47 (d, J = 3.4 Hz, 1H), 8.35- |
| | | | 8.08 (m, 1H), 7.86 (br dd, J = 4.8, 9.4 Hz, 2H), 7.65 (br s, 1H), |
| | | | 7.43-7.17 (m, 1H), 6.89 (t, J = 9.3 Hz, 1H), 6.64-6.42 (m, 2H), |
| | | | 4.29 (br dd, J = 4.8, 11.4 Hz, 1H), 4.10 (br d, J = 12.4 Hz, 2H), |
| | | | 3.70-3.58 (m, 3H), 3.48 (br s, 4H), 3.42-3.15 (m, 9H), 3.00 (br |
| | | | t, J = 11.1 Hz, 1H), 2.74 (ddd, J = 5.3, 12.1, 17.6 Hz, 1H), 2.65- |
| | | | 2.54 (m, 2H), 2.16-1.26 (m, 21H) |
| 494 | 833 | 834 | δ (DMSO-d6) 10.82-10.76 (m, 1H), 8.42 (d, J = 3.7 Hz, 1H), |
| | | | 7.86 (s, 1H), 7.85-7.81 (m, 1H), 7.62 (br d, J = 7.7 Hz, 1H), 7.58- |
| | | | 7.52 (m, 1H), 7.47-7.43 (m, 1H), 7.20 (br d, J = 7.7 Hz, 1H), |
| | | | 6.84 (t, J = 9.4 Hz, 1H), 6.51 (dd, J = 15.0, 2.3 Hz, 1H), 6.41 (dd, |
| | | | J = 8.7, 2.1 Hz, 1H), 5.89 (d, J = 7.8 Hz, 1H), 4.31-4.23 (m, 1H), |
| | | | 3.68-3.60 (m, 3H), 3.49 (br dd, J = 5.2, 1.9 Hz, 1H), 3.29-3.17 |
| | | | (m, 2H), 3.00-2.83 (m, 4H), 2.78-2.66 (m, 2H), 2.58 (br d, J = |
| | | | 4.0 Hz, 1H), 2.57-2.52 (m, 2H), 2.41 (t, J = 6.2 Hz, 2H), 2.11- |
| | | | 2.03 (m, 2H), 2.03-1.94 (m, 2H), 1.93-1.79 (m, 7H), 1.76-1.63 |
| | | | (m, 4H), 1.51-1.39 (m, 2H), 1.37 (br s, 4H) |
| 495 | 835 | 836 | δ (DMSO-d6) 10.78 (br d, J = 4.5 Hz, 1H), 8.43 (d, J = 3.6 Hz, |
| | | | 1H), 7.95 (s, 1H), 7.88-7.81 (m, 1H), 7.63-7.58 (m, 1H), 7.57- |
| | | | 7.51 (m, 2H), 7.18 (br d, J = 7.9 Hz, 1H), 6.88-6.81 (m, 1H), |
| | | | 6.51 (dd, J = 14.8, 2.3 Hz, 1H), 6.42 (br d, J = 9.1 Hz, 1H), 5.88 |
| | | | (d, J = 7.7 Hz, 1H), 4.37 (t, J = 5.3 Hz, 2H), 4.27 (br dd, J = 7.8, |
| | | | 3.3 Hz, 1H), 3.71 (t, J = 6.0 Hz, 2H), 3.67-3.60 (m, 1H), 3.54- |
| | | | 3.44 (m, 1H), 3.25-3.16 (m, 2H), 3.00-2.85 (m, 4H), 2.77-2.66 |
| | | | (m, 2H), 2.58 (br d, J = 4.1 Hz, 1H), 2.57-2.53 (m, 2H), 2.16- |
| | | | 2.08 (m, 3H), 2.08-2.04 (m, 1H), 2.02-1.95 (m, 2H), 1.94-1.78 |
| | | | (m, 3H), 1.77-1.64 (m, 4H), 1.51-1.39 (m, 2H), 1.38-1.22 (m, |
| | | | 4H) |
| 496 | 835 | 836 | δ (DMSO-d6) 10.78 (s, 1H), 8.43 (d, J = 3.6 Hz, 1H), 7.99 (s, |
| | | | 1H), 7.87 (br d, J = 6.1 Hz, 1H), 7.66-7.53 (m, 3H), 7.19 (br d, |
| | | | J = 7.6 Hz, 1H), 6.86 (br s, 1H), 6.52 (br d, J = 14.7 Hz, 1H), 6.43 |
| | | | (br d, J = 8.0 Hz, 1H), 5.94 (br dd, J = 2.7, 7.7 Hz, 1H), 4.30- |
| | | | 4.22 (m, 3H), 4.02-3.98 (m, 2H), 3.79 (br t, J = 4.9 Hz, 2H), 3.70- |
| | | | 3.56 (m, 2H), 3.50 (br d, J = 1.0 Hz, 1H), 3.26 (br d, J = 5.4 Hz, |
| | | | 2H), 3.05-2.83 (m, 3H), 2.73 (ddd, J = 17.6, 12.1, 5.2 Hz, 2H), |
| | | | 2.63-2.53 (m, 2H), 2.07 (dt, J = 8.5, 4.5 Hz, 3H), 2.00 (br d, J = |
| | | | 9.5 Hz, 3H), 1.90-1.81 (m, 2H), 1.80-1.67 (m, 4H), 1.54-1.39 |
| | | | (m, 3H), 1.35-1.19 (m, 3H) |
| 497 | 776 | 777 | δ (DMSO-d6) 10.92-10.59 (m, 1H), 8.41 (d, J = 3.7 Hz, 1H), |
| | | | 7.75-7.66 (m, 2H), 7.63 (d, J = 7.8 Hz, 1H), 7.42 (t, J = 7.7 Hz, |
| | | | 1H), 7.26 (br d, J = 7.6 Hz, 1H), 7.17 (d, J = 7.7 Hz, 1H), 6.86 (t, |
| | | | J = 9.4 Hz, 1H), 6.57-6.48 (m, 1H), 6.47-6.39 (m, 1H), 5.91 (d, |
| | | | J = 7.8 Hz, 1H), 4.33-4.25 (m, 1H), 3.70-3.59 (m, 1H), 3.58- |
| | | | 3.47 (m, 1H), 3.29-3.17 (m, 2H), 3.03 (br d, J = 11.1 Hz, 1H), |
| | | | 3.00-2.93 (m, 2H), 2.92-2.83 (m, 1H), 2.80-2.68 (m, 2H), 2.63- |
| | | | 2.54 (m, 2H), 2.13-2.04 (m, 2H), 2.03-1.91 (m, 4H), 1.90- |
| | | | 1.84 (m, 1H), 1.83-1.75 (m, 3H), 1.66-1.48 (m, 4H), 1.42-1.22 |
| | | | (m, 5H), 1.05-0.99 (m, 2H), 0.75-0.69 (m, 2H) |
| 498 | 819 | 820 | δ (DMSO-d6) 10.93-10.64 (m, 1H), 8.38 (d, J = 3.9 Hz, 1H), |
| | | | 7.60 (br d, J = 7.9 Hz, 1H), 7.55 (br d, J = 1.6 Hz, 1H), 7.34 (br |
| | | | d, J = 5.6 Hz, 2H), 7.11 (br d, J = 7.8 Hz, 2H), 6.85 (t, J = 9.4 Hz, |
| | | | 1H), 6.52 (dd, J = 14.9, 2.3 Hz, 1H), 6.45-6.40 (m, 1H), 5.89 (d, |
| | | | J = 7.5 Hz, 1H), 4.33-4.23 (m, 1H), 3.62 (br dd, J = 6.6, 3.4 Hz, |
| | | | 1H), 3.55-3.46 (m, 1H), 3.27-3.18 (m, 6H), 3.03 (br d, J = 11.5 |
| | | | Hz, 1H), 2.96 (br dd, J = 11.4, 5.1 Hz, 2H), 2.91-2.83 (m, 1H), |
| | | | 2.78-2.67 (m, 2H), 2.62-2.57 (m, 1H), 2.57-2.53 (m, 1H), 2.12- |
| | | | 2.04 (m, 2H), 2.02-1.94 (m, 2H), 1.93-1.87 (m, 1H), 1.86- |
| | | | 1.74 (m, 4H), 1.64 (br d, J = 4.4 Hz, 5H), 1.60-1.50 (m, 5H), |
| | | | 1.41-1.18 (m, 5H) |
| 499 | 833 | 834 | δ (DMSO-d6) 10.78 (s, 1H), 8.45-8.38 (m, 1H), 7.86 (s, 1H), |
| | | | 7.83 (br d, J = 7.5 Hz, 1H), 7.62-7.57 (m, 1H), 7.54 (t, J = 7.9 |
| | | | Hz, 1H), 7.45 (br d, J = 8.8 Hz, 1H), 7.18 (br d, J = 8.0 Hz, 1H), |
| | | | 6.84 (br t, J = 9.2 Hz, 1H), 6.51 (br d, J = 15.1 Hz, 1H), 6.42 (br |
| | | | d, J = 8.9 Hz, 1H), 5.88 (br d, J = 7.4 Hz, 1H), 4.31-4.23 (m, |
| | | | 1H), 3.71-3.55 (m, 3H), 3.55-3.44 (m, 1H), 3.28-3.14 (m, 2H), |
| | | | 3.07-2.81 (m, 4H), 2.78-2.66 (m, 2H), 2.58 (br d, J = 4.4 Hz, |
| | | | 1H), 2.54 (br dd, J = 7.7, 3.3 Hz, 1H), 2.42 (br t, J = 6.2 Hz, 2H), |
| | | | 2.02 (br s, 2H), 1.95 (br t, J = 8.4 Hz, 3H), 1.92-1.82 (m, 6H), |
| | | | 1.78 (br dd, J = 5.1, 4.2 Hz, 3H), 1.66-1.46 (m, 4H), 1.39-1.21 |
| | | | (m, 4H) |
| 500 | 835 | 836 | δ (DMSO-d6) 10.86-10.75 (m, 1H), 8.45 (d, J = 3.5 Hz, 1H), |
| | | | 7.99-7.93 (m, 1H), 7.85 (br d, J = 6.1 Hz, 1H), 7.67-7.60 (m, |
| | | | 1H), 7.60-7.51 (m, 2H), 7.26-7.17 (m, 1H), 6.86 (t, J = 9.3 Hz, |
| | | | 1H), 6.58-6.49 (m, 1H), 6.47-6.39 (m, 1H), 5.91 (d, J = 7.7 Hz, |
| | | | 1H), 4.38 (t, J = 5.3 Hz, 2H), 4.33-4.25 (m, 1H), 3.72 (br t, J = |
| | | | 5.9 Hz, 2H), 3.68-3.58 (m, 1H), 3.56-3.47 (m, 1H), 3.30-3.17 |
| | | | (m, 2H), 3.03 (br d, J = 10.6 Hz, 1H), 3.00-2.93 (m, 2H), 2.92- |
| | | | 2.84 (m, 1H), 2.79-2.69 (m, 2H), 2.63-2.53 (m, 3H), 2.18-2.11 |
| | | | (m, 2H), 2.10-2.02 (m, 2H), 2.01-1.90 (m, 3H), 1.89-1.84 (m, |
| | | | 1H), 1.82-1.75 (m, 3H), 1.67-1.48 (m, 4H), 1.41-1.21 (m, 4H) |
| 501 | 835 | 836 | δ (DMSO-d6) 10.77 (s, 1H), 8.43 (d, J = 3.6 Hz, 1H), 7.99 (s, |
| | | | 1H), 7.86 (br d, J = 5.1 Hz, 1H), 7.64-7.53 (m, 3H), 7.19 (br d, |
| | | | J = 7.8 Hz, 1H), 6.84 (t, J = 9.4 Hz, 1H), 6.55-6.46 (m, 1H), 6.42 |
| | | | (dd, J = 8.8, 1.8 Hz, 1H), 5.88 (br d, J = 7.5 Hz, 1H), 4.31-4.22 |
| | | | (m, 3H), 4.02-3.98 (m, 2H), 3.78 (t, J = 5.0 Hz, 2H), 3.70-3.57 |
| | | | (m, 1H), 3.50 (br dd, J = 7.4, 3.4 Hz, 1H), 3.28-3.16 (m, 2H), |
| | | | 3.05-2.82 (m, 4H), 2.78-2.68 (m, 2H), 2.61-2.52 (m, 2H), 2.08 |
| | | | (td, J = 4.2, 8.4 Hz, 2H), 1.95 (br s, 3H), 1.90-1.74 (m, 5H), 1.65- |
| | | | 1.46 (m, 4H), 1.39-1.22 (m, 4H) |
| 502 | 758 | 759 | δ (DMSO-d6) 10.80 (br s, 1H), 8.41 (d, J = 3.7 Hz, 1H), 7.74- |
| | | | 7.68 (m, 2H), 7.63 (d, J = 7.7 Hz, 1H), 7.42 (t, J = 7.6 Hz, 1H), |
| | | | 7.26 (br d, J = 7.8 Hz, 1H), 7.17 (d, J = 7.6 Hz, 1H), 6.83 (t, J = |
| | | | 9.3 Hz, 1H), 6.52 (dd, J = 15.2, 2.3 Hz, 1H), 6.42 (dd, J = 8.9, 1.8 |
| | | | Hz, 1H), 5.84 (d, J = 7.7 Hz, 1H), 5.09-4.93 (m, 1H), 4.31-4.24 |
| | | | (m, 1H), 3.68-3.60 (m, 1H), 3.56-3.48 (m, 1H), 3.22 (br d, J = |
| | | | 11.2 Hz, 1H), 3.03-2.96 (m, 2H), 2.84-2.75 (m, 1H), 2.73-2.65 |
| | | | (m, 2H), 2.60 (t, J = 4.1 Hz, 1H), 2.57-2.54 (m, 1H), 2.43-2.33 |
| | | | (m, 1H), 2.30-2.21 (m, 2H), 2.12-1.97 (m, 6H), 1.90-1.79 (m, |
| | | | 3H), 1.69-1.52 (m, 5H), 1.38-1.25 (m, 4H), 1.05-0.99 (m, 2H), |
| | | | 0.75-0.71 (m, 2H) |
| 503 | 739 | 740 | δ 10.79 (s, 1H), 10.19 (br d, J = 6.4 Hz, 1H), 8.39 (d, J = 3.5 Hz, |
| | | | 1H), 7.74-7.65 (m, 3H), 7.41 (br t, J = 7.6 Hz, 1H), 7.24 (br d, |
| | | | J = 8.0 Hz, 1H), 6.96 (br t, J = 8.4 Hz, 1H), 6.54-6.43 (m, 2H), |
| | | | 4.32 (br dd, J = 4.6, 11.8 Hz, 1H), 3.20-3.04 (m, 4H), 3.01-2.90 |
| | | | (m, 1H), 2.79-2.66 (m, 1H), 2.57 (br d, J = 17.4 Hz, 1H), 2.16- |
| | | | 1.97 (m, 9H), 1.88 (br d, J = 10.0 Hz, 5H), 1.74 (br d, J = 11.4 |
| | | | Hz, 2H), 1.58 (br d, J = 10.5 Hz, 2H), 1.51-1.38 (m, 3H), 1.35- |
| | | | 1.16 (m, 5H), 1.04-0.95 (m, 2H), 0.86 (dt, J = 3.8, 7.2 Hz, 1H), |
| | | | 0.74-0.66 (m, 2H) |
| 504 | 740 | 741 | δ 10.84-10.69 (m, 1H), 8.39 (d, J = 3.8 Hz, 1H), 7.75-7.65 (m, |
| | | | 2H), 7.60 (br d, J = 8.0 Hz, 1H), 7.41 (t, J = 7.6 Hz, 1H), 7.25 (br |
| | | | d, J = 8.0 Hz, 1H), 7.14 (d, J = 8.0 Hz, 1H), 6.82 (t, J = 9.4 Hz, |
| | | | 1H), 6.50 (dd, J = 15.0, 2.4 Hz, 1H), 6.41 (dd, J = 8.8, 2.2 Hz, |
| | | | 1H), 5.79 (d, J = 8.0 Hz, 1H), 4.31-4.19 (m, 1H), 3.71-3.57 (m, |
| | | | 1H), 3.56-3.48 (m, 1H), 3.22-3.10 (m, 2H), 2.90 (br d, J = 11.0 |
| | | | Hz, 2H), 2.79-2.63 (m, 2H), 2.58 (br d, J = 4.2 Hz, 1H), 2.37- |
| | | | 2.24 (m, 1H), 2.18-2.05 (m, 3H), 2.04-1.91 (m, 4H), 1.89-1.72 |
| | | | (m, 5H), 1.68-1.49 (m, 6H), 1.41-1.20 (m, 5H), 1.05-0.97 (m, |
| | | | 2H), 0.77-0.67 (m, 2H) |
| 505 | 828 | 829 | δ (DMSO-d6) 10.88-10.83 (m, 1H), 8.46 (d, J = 3.5 Hz, 1H), |
| | | | 8.05 (br d, J = 8.0 Hz, 1H), 7.98 (br s, 1H), 7.74-7.65 (m, 3H), |
| | | | 7.62-7.59 (m, 1H), 7.57-7.53 (m, 1H), 7.49 (d, J = 9.0 Hz, 1H), |
| | | | 7.22 (br d, J = 7.6 Hz, 1H), 6.96-6.88 (m, 1H), 6.82 (s, 1H), 6.52 |
| | | | (d, J = 9.4 Hz, 1H), 6.37-6.32 (m, 1H), 4.26 (dd, J = 9.0, 5.0 Hz, |
| | | | 1H), 3.89 (s, 3H), 3.67-3.59 (m, 1H), 3.56-3.49 (m, 1H), 3.20 |
| | | | (br s, 3H), 2.67 (br s, 4H), 2.62 (br d, J = 5.0 Hz, 2H), 2.36-2.21 |
| | | | (m, 3H), 2.20-2.10 (m, 1H), 1.98-1.91 (m, 4H), 1.85-1.78 (m, |
| | | | 4H), 1.76-1.71 (m, 2H), 1.65-1.55 (m, 1H), 1.32 (br s, 2H), |
| | | | 1.28-1.20 (m, 4H), 0.96 (br d, J = 12.4 Hz, 2H) |
| 506 | 843 | 844 | δ 10.86 (s, 1H), 8.46 (d, J = 3.6 Hz, 1H), 8.04 (d, J = 7.8 Hz, 1H), |
| | | | 7.96 (s, 1H), 7.91 (br d, J = 7.6 Hz, 1H), 7.74-7.66 (m, 2H), 7.61- |
| | | | 7.56 (m, 2H), 7.56-7.52 (m, 1H), 7.03-6.94 (m, 2H), 6.51 (d, |
| | | | J = 9.3 Hz, 1H), 6.36 (dt, J = 6.7, 1.0 Hz, 1H), 4.29 (dd, J = 9.2, |
| | | | 5.1 Hz, 1H), 3.99 (br s, 1H), 3.93 (s, 3H), 3.82-3.62 (m, 10H), |
| | | | 3.25 (br s, 5H), 3.06-2.90 (m, 2H), 2.67-2.58 (m, 2H), 2.43- |
| | | | 2.26 (m, 2H), 2.21-2.10 (m, 1H), 1.93 (br d, J = 9.4 Hz, 6H), |
| | | | 1.84-1.75 (m, 2H), 1.41-1.18 (m, 4H) |
| 507 | 827 | 828 | δ (DMSO-d6) 10.87 (s, 1H), 8.46 (d, J = 3.5 Hz, 1H), 8.05 (br d, |
| | | | J = 7.3 Hz, 1H), 7.98 (br s, 1H), 7.75-7.69 (m, 2H), 7.69-7.65 |
| | | | (m, 1H), 7.60 (br d, J = 8.9 Hz, 2H), 7.58-7.52 (m, 1H), 7.43 (s, |
| | | | 1H), 7.22 (br d, J = 7.6 Hz, 1H), 7.03 (d, J = 8.4 Hz, 1H), 6.52 (d, |
| | | | J = 9.0 Hz, 1H), 6.36 (t, J = 6.6 Hz, 1H), 4.32 (dd, J = 9.6, 5.1 Hz, |
| | | | 1H), 3.97 (s, 3H), 3.69-3.60 (m, 1H), 3.55-3.48 (m, 1H), 2.95 |
| | | | (br d, J = 9.1 Hz, 2H), 2.66-2.56 (m, 3H), 2.36-2.28 (m, 4H), |
| | | | 2.21-2.15 (m, 1H), 1.97-1.90 (m, 4H), 1.80 (br d, J = 11.0 Hz, |
| | | | 6H), 1.76-1.70 (m, 4H), 1.61-1.55 (m, 1H), 1.33 (br d, J = 13.4 |
| | | | Hz, 2H), 1.27 (br d, J = 13.5 Hz, 4H), 0.95 (br d, J = 11.9 Hz, 2H) |
| 508 | 799 | 800 | δ 10.81 (s, 1H), 8.45 (d, J = 3.4 Hz, 1H), 8.15 (s, 0.14H), 8.01 (s, |
| | | | 1H), 7.89 (br d, J = 5.6 Hz, 1H), 7.72-7.55 (m, 3H), 7.22 (br d, |
| | | | J = 7.8 Hz, 1H), 6.87 (br t, J = 9.4 Hz, 1H), 6.62-6.51 (m, 1H), |
| | | | 6.45 (br d, J = 8.8 Hz, 1H), 5.99-5.83 (m, 1H), 4.35-4.27 (m, |
| | | | 1H), 4.26 (s, 2H), 4.02 (t, J = 5.0 Hz, 2H), 3.81 (t, J = 4.8 Hz, |
| | | | 2H), 3.72-3.61 (m, 1H), 3.56-3.45 (m, 2H), 3.10-2.86 (m, 6H), |
| | | | 2.82-2.68 (m, 2H), 2.63-2.57 (m, 1H), 2.14-1.96 (m, 6H), 1.93- |
| | | | 1.83 (m, 3H), 1.75 (br d, J = 11.4 Hz, 2H), 1.55-1.37 (m, 4H), |
| | | | 1.35-1.11 (m, 5H) |
| 509 | 813 | 814 | δ 10.81 (s, 1H), 8.46 (d, J = 3.4 Hz, 1H), 8.19-8.01 (m, 2H), 7.76- |
| | | | 7.67 (m, 2H), 7.67-7.60 (m, 1H), 7.25 (br d, J = 7.6 Hz, 1H), |
| | | | 7.00-6.83 (m, 1H), 6.63-6.51 (m, 1H), 6.50-6.37 (m, 1H), 6.08- |
| | | | 5.84 (m, 1H), 5.11-4.93 (m, 1H), 4.42-4.21 (m, 2H), 3.67- |
| | | | 3.50 (m, 7H), 3.29-3.12 (m, 3H), 3.06-2.89 (m, 2H), 2.84-2.65 |
| | | | (m, 2H), 2.64-2.54 (m, 2H), 2.12-2.05 (m, 3H), 2.05-1.98 (m, |
| | | | 3H), 1.96-1.80 (m, 5H), 1.80-1.67 (m, 2H), 1.63-1.38 (m, 4H), |
| | | | 1.33-1.19 (m, 4H), 1.15-1.00 (m, 1H) |
| 510 | 812 | 813 | δ 10.79 (s, 1H), 8.44 (d, J = 3.6 Hz, 1H), 8.40 (br s, 0.43H), 8.19- |
| | | | 8.01 (m, 2H), 7.73-7.53 (m, 3H), 7.21 (br d, J = 7.4 Hz, 1H), |
| | | | 7.05-6.91 (m, 1H), 6.49-6.37 (m, 2H), 5.99 (br d, J = 8.0 Hz, |
| | | | 1H), 5.12-4.89 (m, 1H), 4.42-4.19 (m, 2H), 3.68-3.53 (m, 4H), |
| | | | 2.92-2.85 (m, 2H), 2.82-2.64 (m, 2H), 2.60-2.57 (m, 1H), 2.30- |
| | | | 2.21 (m, 3H), 2.07 (td, J = 8.2, 4.0 Hz, 2H), 2.04-1.95 (m, 3H), |
| | | | 1.84-1.71 (m, 7H), 1.67-1.54 (m, 4H), 1.53-1.37 (m, 3H), 1.36- |
| | | | 1.09 (m, 8H) |
| 511 | 812 | 813 | δ 10.81 (s, 1H), 8.46 (d, J = 3.6 Hz, 1H), 8.20 (s, 0.63H), 8.12 (br |
| | | | s, 1H), 8.06 (br d, J = 7.6 Hz, 1H), 7.74-7.67 (m, 1H), 7.66- |
| | | | 7.57 (m, 2H), 7.24 (br d, J = 7.2 Hz, 1H), 7.03-6.94 (m, 1H), |
| | | | 6.51-6.39 (m, 2H), 6.03 (d, J = 7.6 Hz, 1H), 4.39-4.24 (m, 2H), |
| | | | 3.65-3.56 (m, 4H), 3.44 (br s, 1H), 3.40 (br s, 1H), 3.23 (br d, |
| | | | J = 10.6 Hz, 1H), 3.00 (br d, J = 9.8 Hz, 2H), 2.80-2.71 (m, 1H), |
| | | | 2.60 (br t, J = 3.6 Hz, 2H), 2.56 (br dd, J = 8.0, 4.0 Hz, 1H), 2.45 |
| | | | (br d, J = 7.8 Hz, 2H), 2.13-2.05 (m, 1H), 2.01-1.93 (m, 3H), |
| | | | 1.93-1.85 (m, 4H), 1.84-1.74 (m, 5H), 1.73-1.60 (m, 4H), 1.46- |
| | | | 1.21 (m, 8H) |
| 512 | 832 | 833 | δ 10.56 (s, 1H), 8.46 (s, 1.08H), 8.20 (s, 1H), 8.05 (br d, J = 7.6 |
| | | | Hz, 1H), 7.98 (br s, 1H), 7.75-7.67 (m, 2H), 7.63-7.52 (m, 4H), |
| | | | 7.37 (d, J = 10.8 Hz, 1H), 7.23 (br d, J = 7.6 Hz, 1H), 6.52 (d, J = |
| | | | 9.4 Hz, 1H), 6.36 (t, J = 6.8 Hz, 1H), 4.00 (s, 3H), 3.91 (br t, J = |
| | | | 6.6 Hz, 2H), 3.69-3.63 (m, 2H), 3.08-2.98 (m, 2H), 2.92-2.83 |
| | | | (m, 1H), 2.81-2.69 (m, 2H), 2.43 (br d, J = 9.8 Hz, 3H), 2.05- |
| | | | 1.92 (m, 3H), 1.91-1.74 (m, 10H), 1.48-1.18 (m, 8H) |
| 513 | 783 | 784 | δ 10.77 (s, 1H), 8.37 (d, J = 3.9 Hz, 1H), 8.13 (s, 0.5H), 7.64- |
| | | | 7.43 (m, 1H), 7.34 (br d, J = 4.5 Hz, 2H), 7.20-7.06 (m, 2H), |
| | | | 6.83 (t, J = 9.3 Hz, 1H), 6.51 (dd, J = 2.3, 14.9 Hz, 1H), 6.42 (dd, |
| | | | J = 2.3, 8.7 Hz, 1H), 5.80 (br d, J = 7.6 Hz, 1H), 4.43-4.32 (m, |
| | | | 1H), 4.25 (ddd, J = 4.7, 7.3, 11.7 Hz, 1H), 3.94 (br d, J = 12.3 Hz, |
| | | | 1H), 3.70-3.54 (m, 1H), 3.25-3.15 (m, 5H), 3.05-2.94 (m, 2H), |
| | | | 2.86 (br s, 4H), 2.72 (dt, J = 6.1, 12.0 Hz, 2H), 2.21 (br s, 2H), |
| | | | 2.15-1.92 (m, 4H), 1.90-1.26 (m, 19H), 1.08-0.85 (m, 2H) |
| 514 | 798 | 799 | δ 11.05-10.45 (m, 1H), 8.38 (d, J = 3.8 Hz, 1H), 7.65-7.47 (m, |
| | | | 1H), 7.34 (br d, J = 4.4 Hz, 2H), 7.18 (d, J = 7.5 Hz, 1H), 7.14- |
| | | | 7.08 (m, 1H), 6.98 (t, J = 8.8 Hz, 1H), 6.48-6.39 (m, 2H), 5.98 |
| | | | (d, J = 7.6 Hz, 1H), 4.36-4.13 (m, 2H), 4.05 (br d, J = 12.0 Hz, |
| | | | 1H), 3.76-3.57 (m, 2H), 3.24-3.13 (m, 5H), 3.07-2.89 (m, 3H), |
| | | | 2.80-2.65 (m, 1H), 2.56 (br dd, J = 3.9, 13.2 Hz, 3H), 2.35-2.17 |
| | | | (m, 4H), 2.14-1.96 (m, 3H), 1.85 (dq, J = 4.8, 12.4 Hz, 1H), 1.72 |
| | | | (br d, J = 13.0 Hz, 2H), 1.68-1.31 (m, 18H) |
| 515 | 782 | 783 | δ 11.06-10.55 (m, 1H), 8.38 (d, J = 3.9 Hz, 1H), 7.56 (br d, J = |
| | | | 7.8 Hz, 2H), 7.34 (br d, J = 4.8 Hz, 2H), 7.21-7.05 (m, 2H), 7.04- |
| | | | 6.87 (m, 1H), 6.49-6.37 (m, 2H), 5.98 (d, J = 7.8 Hz, 1H), 4.34- |
| | | | 4.22 (m, 1H), 3.71-3.55 (m, 1H), 3.26-3.17 (m, 5H), 2.89 (br |
| | | | d, J = 10.8 Hz, 2H), 2.80-2.65 (m, 1H), 2.63-2.54 (m, 2H), 2.36- |
| | | | 2.17 (m, 3H), 2.15-1.96 (m, 4H), 1.91-1.69 (m, 7H), 1.68- |
| | | | 1.50 (m, 10H), 1.49-1.39 (m, 2H), 1.37-1.09 (m, 6H) |
| 516 | 783 | 784 | δ 10.77 (s, 1H), 8.37 (d, J = 3.8 Hz, 1H), 8.15 (s, 0.5H), 7.64 (br |
| | | | d, J = 7.8 Hz, 1H), 7.54 (br s, 1H), 7.33 (br d, J = 5.9 Hz, 2H), |
| | | | 7.11 (br d, J = 7.8 Hz, 2H), 6.82 (t, J = 9.4 Hz, 1H), 6.57-6.33 |
| | | | (m, 2H), 5.79 (d, J = 7.6 Hz, 1H), 4.25 (ddd, J = 4.8, 7.3, 11.7 Hz, |
| | | | 1H), 3.61 (br dd, J = 3.4, 7.6 Hz, 1H), 3.53-3.48 (m, 1H), 3.22- |
| | | | 3.12 (m, 7H), 2.99 (br d, J = 10.5 Hz, 2H), 2.79-2.65 (m, 1H), |
| | | | 2.61-2.54 (m, 2H), 2.44 (br s, 1H), 2.26 (br t, J = 10.3 Hz, 2H), |
| | | | 2.13-2.04 (m, 2H), 1.98 (br d, J = 2.0 Hz, 2H), 1.80 (br d, J = |
| | | | 9.8 Hz, 5H), 1.72-1.44 (m, 12H), 1.41-1.16 (m, 4H) |
| 517 | 793 | 794 | δ 12.24-11.51 (m, 1H), 10.77 (s, 1H), 8.41 (d, J = 3.4 Hz, 1H), |
| | | | 8.31 (s, 1H), 8.16 (s, 0.3H), 7.94-7.80 (m, 2H), 7.70 (br d, J = |
| | | | 6.9 Hz, 1H), 7.63-7.48 (m, 2H), 7.43 (br d, J = 6.0 Hz, 1H), 7.15 |
| | | | (br d, J = 7.5 Hz, 1H), 6.81 (br t, J = 9.3 Hz, 1H), 6.50 (br d, J = |
| | | | 15.3 Hz, 1H), 6.41 (br d, J = 8.3 Hz, 1H), 6.32 (t, J = 6.6 Hz, 1H), |
| | | | 5.79 (br d, J = 6.8 Hz, 1H), 4.31-4.18 (m, 1H), 3.75-3.40 (m, |
| | | | 3H), 2.84 (br s, 3H), 2.79-2.56 (m, 6H), 2.28 (br t, J = 10.3 Hz, |
| | | | 1H), 2.15-1.66 (m, 11H), 1.44-1.13 (m, 8H) |
| 518 | 755 | 756 | δ 10.79 (s, 1H), 8.40 (d, J = 3.6 Hz, 1H), 8.13 (s, 0.1H), 7.81- |
| | | | 7.60 (m, 2H), 7.40 (t, J = 7.8 Hz, 1H), 7.30-7.16 (m, 2H), 6.96 |
| | | | (br s, 1H), 6.56-6.41 (m, 2H), 6.08 (br d, J = 7.6 Hz, 1H), 5.60- |
| | | | 4.95 (m, 1H), 4.32 (ddd, J = 4.9, 7.4, 11.9 Hz, 1H), 4.10 (br d, |
| | | | J = 12.0 Hz, 1H), 3.88-3.50 (m, 3H), 3.43-3.35 (m, 1H), 3.18 (br |
| | | | d, J = 5.1 Hz, 2H), 3.01-2.86 (m, 2H), 2.85-2.63 (m, 2H), 2.63- |
| | | | 2.52 (m, 2H), 2.15-1.95 (m, 6H), 1.93-1.80 (m, 2H), 1.79- |
| | | | 1.60 (m, 6H), 1.59-1.39 (m, 5H), 1.37-1.20 (m, 2H), 1.05-0.94 |
| | | | (m, 2H), 0.77-0.65 (m, 2H) |
| 519 | 799 | 800 | δ 10.79 (s, 1H), 8.46 (d, J = 3.6 Hz, 1H), 8.04-7.94 (m, 2H), 7.86 |
| | | | (br s, 1H), 7.59 (br d, J = 7.5 Hz, 1H), 7.22 (br d, J = 7.6 Hz, 1H), |
| | | | 6.98 (br t, J = 8.7 Hz, 1H), 6.51-6.38 (m, 2H), 6.10-5.90 (m, |
| | | | 1H), 4.37 (t, J = 5.4 Hz, 2H), 4.33-4.25 (m, 1H), 4.00 (t, J = 6.1 |
| | | | Hz, 2H), 3.75-3.56 (m, 1H), 3.50-3.40 (m, 2H), 3.10-2.92 (m, |
| | | | 2H), 2.80-2.63 (m, 2H), 2.61-2.53 (m, 2H), 2.22-1.94 (m, 6H), |
| | | | 1.93-1.77 (m, 6H), 1.77-1.58 (m, 6H), 1.55-0.99 (m, 9H). |
| 520 | 793 | 794 | δ = 10.94-10.56 (m, 1H), 8.49 (d, J = 3.4 Hz, 1H), 8.25-8.04 |
| | | | (m, 2H), 8.01-7.88 (m, 2H), 7.66-7.45 (m, 2H), 7.29 (br d, J = |
| | | | 7.8 Hz, 1H), 7.09-6.90 (m, 1H), 6.55 (d, J = 9.3 Hz, 1H), 6.48- |
| | | | 6.34 (m, 3H), 5.97 (d, J = 7.4 Hz, 1H), 4.37-4.19 (m, 1H), 3.77- |
| | | | 3.56 (m, 1H), 2.88 (br d, J = 10.9 Hz, 2H), 2.80-2.55 (m, 3H), |
| | | | 2.29-2.18 (m, 3H), 2.12-1.94 (m, 4H), 1.87-1.71 (m, 7H), 1.68- |
| | | | 1.51 (m, 5H), 1.49-1.38 (m, 2H), 1.35-1.07 (m, 7H) |
| 521 | 797 | 798 | δ (DMSO-d6) 10.84-10.70 (m, 1H), 8.43 (d, J = 3.6 Hz, 1H), |
| | | | 7.88-7.83 (m, 2H), 7.62-7.53 (m, 2H), 7.46 (br d, J = 8.9 Hz, |
| | | | 1H), 7.19 (br d, J = 7.9 Hz, 1H), 6.86-6.80 (m, 1H), 6.50 (dd, |
| | | | J = 15.0, 2.1 Hz, 1H), 6.43-6.39 (m, 1H), 5.79 (d, J = 7.6 Hz, 1H), |
| | | | 4.29-4.22 (m, 1H), 3.65 (br t, J = 5.5 Hz, 3H), 3.49 (br dd, J = |
| | | | 11.9, 4.6 Hz, 1H), 3.16 (br d, J = 10.8 Hz, 2H), 2.83 (br d, J = |
| | | | 11.3 Hz, 2H), 2.77-2.68 (m, 1H), 2.59 (br d, J = 4.1 Hz, 1H), |
| | | | 2.55 (br s, 1H), 2.43 (t, J = 6.2 Hz, 2H), 2.33-2.27 (m, 1H), 2.20 |
| | | | (br t, J = 10.3 Hz, 2H), 2.10 (br d, J = 4.1 Hz, 1H), 2.09-2.06 (m, |
| | | | 1H), 2.03-1.97 (m, 2H), 1.88 (br dd, J = 10.9, 6.0 Hz, 5H), 1.78- |
| | | | 1.69 (m, 6H), 1.60-1.51 (m, 3H), 1.48-1.40 (m, 2H), 1.36- |
| | | | 1.25 (m, 4H) |
| 522 | 799 | 800 | δ (DMSO-d6) 10.77 (br s, 1H), 8.43 (d, J = 3.6 Hz, 1H), 7.95 (s, |
| | | | 1H), 7.85 (br d, J = 6.6 Hz, 1H), 7.63-7.51 (m, 3H), 7.19 (br d, |
| | | | J = 8.0 Hz, 1H), 6.83 (t, J = 9.4 Hz, 1H), 6.50 (dd, J = 2.3, 14.9 |
| | | | Hz, 1H), 6.41 (dd, J = 2.3, 8.6 Hz, 1H), 5.78 (d, J = 7.5 Hz, 1H), |
| | | | 4.37 (t, J = 5.3 Hz, 2H), 4.30-4.21 (m, 1H), 3.75-3.60 (m, 3H), |
| | | | 3.54-3.43 (m, 1H), 3.16 (br d, J = 10.6 Hz, 2H), 2.83 (br d, J = |
| | | | 11.3 Hz, 2H), 2.78-2.65 (m, 1H), 2.60-2.54 (m, 2H), 2.36-2.26 |
| | | | (m, 1H), 2.24-2.16 (m, 2H), 2.16-2.05 (m, 4H), 2.00 (br d, J = |
| | | | 9.6 Hz, 2H), 1.91-1.81 (m, 1H), 1.78-1.67 (m, 6H), 1.61-1.24 |
| | | | (m, 9H) |
| 523 | 801 | 802 | δ (DMSO-d6) 10.81-10.75 (m, 1H), 8.38 (d, J = 3.8 Hz, 1H), |
| | | | 7.60 (br d, J = 7.9 Hz, 1H), 7.58-7.51 (m, 1H), 7.34 (br d, J = |
| | | | 5.6 Hz, 2H), 7.11 (br d, J = 7.9 Hz, 2H), 6.82 (t, J = 9.3 Hz, 1H), |
| | | | 6.51 (dd, J = 2.4, 15.1 Hz, 1H), 6.42 (dd, J = 2.2, 8.8 Hz, 1H), |
| | | | 5.82 (d, J = 7.6 Hz, 1H), 5.00 (br dd, J = 2.9, 4.6 Hz, 1H), 4.30- |
| | | | 4.22 (m, 1H), 3.68-3.57 (m, 1H), 3.56-3.45 (m, 1H), 3.20 (br t, |
| | | | J = 5.0 Hz, 6H), 2.99 (br d, J = 6.5 Hz, 2H), 2.79-2.62 (m, 3H), |
| | | | 2.30-2.19 (m, 2H), 2.13-1.92 (m, 6H), 1.84-1.76 (m, 3H), 1.69- |
| | | | 1.60 (m, 8H), 1.59-1.54 (m, 3H), 1.40-1.19 (m, 5H) |
| 524 | 815 | 816 | δ (DMSO-d6) 10.78 (s, 1H), 8.43 (d, J = 3.6 Hz, 1H), 8.14 (s, |
| | | | 1H), 7.87 (s, 1H), 7.84 (br d, J = 7.4 Hz, 1H), 7.71-7.61 (m, 1H), |
| | | | 7.60-7.51 (m, 1H), 7.46 (br d, J = 7.8 Hz, 1H), 7.19 (br d, J = |
| | | | 7.5 Hz, 1H), 6.83 (t, J = 9.3 Hz, 1H), 6.51 (dd, J = 15.1, 2.1 Hz, |
| | | | 1H), 6.46-6.39 (m, 1H), 5.84 (br d, J = 7.3 Hz, 1H), 5.20-4.93 |
| | | | (m, 1H), 4.34-4.22 (m, 1H), 3.65 (br t, J = 5.3 Hz, 3H), 3.54- |
| | | | 3.47 (m, 1H), 3.23 (br d, J = 11.1 Hz, 2H), 3.19-2.97 (m, 3H), |
| | | | 2.79-2.66 (m, 3H), 2.57-2.53 (m, 1H), 2.43 (br t, J = 6.3 Hz, |
| | | | 2H), 2.09 (td, J = 4.1, 8.4 Hz, 2H), 2.02-1.94 (m, 3H), 1.93- |
| | | | 1.83 (m, 6H), 1.82-1.77 (m, 2H), 1.73-1.56 (m, 4H), 1.38-1.21 |
| | | | (m, 6H) |
| 525 | 817 | 818 | δ (DMSO-d6) 10.80 (s, 1H), 8.45 (d, J = 3.5 Hz, 1H), 8.16 (s, |
| | | | 1H), 7.96 (s, 1H), 7.85 (br d, J = 6.0 Hz, 1H), 7.65 (br d, J = 7.8 |
| | | | Hz, 1H), 7.60-7.53 (m, 2H), 7.21 (br d, J = 7.7 Hz, 1H), 6.83 (t, |
| | | | J = 9.4 Hz, 1H), 6.52 (dd, J = 15.0, 2.2 Hz, 1H), 6.43 (dd, J = 8.8, |
| | | | 1.8 Hz, 1H), 5.85 (d, J = 7.6 Hz, 1H), 5.12-4.96 (m, 1H), 4.39 |
| | | | (t, J = 5.3 Hz, 2H), 4.31-4.24 (m, 1H), 3.73 (t, J = 6.1 Hz, 2H), |
| | | | 3.69-3.63 (m, 1H), 3.55-3.47 (m, 2H), 3.41-3.32 (m, 3H), 3.23 |
| | | | (br d, J = 11.0 Hz, 1H), 3.09-3.01 (m, 2H), 2.87-2.77 (m, 1H), |
| | | | 2.77-2.68 (m, 2H), 2.66-2.55 (m, 2H), 2.32 (br d, J = 8.9 Hz, |
| | | | 1H), 2.17-2.07 (m, 4H), 2.01-1.96 (m, 2H), 1.92-1.84 (m, 1H), |
| | | | 1.84-1.78 (m, 2H), 1.71-1.58 (m, 4H), 1.40-1.24 (m, 4H) |
| 526 | 817 | 818 | δ (DMSO-d6) 10.78 (s, 1H), 8.44 (d, J = 3.5 Hz, 1H), 8.14 (s, |
| | | | 1H), 8.00 (s, 1H), 7.87 (br d, J = 4.8 Hz, 1H), 7.74-7.64 (m, 1H), |
| | | | 7.61-7.56 (m, 2H), 7.20 (br d, J = 7.8 Hz, 1H), 6.87-6.80 (m, |
| | | | 1H), 6.52 (dd, J = 2.3, 15.0 Hz, 1H), 6.45-6.40 (m, 1H), 5.85 (br |
| | | | d, J = 7.3 Hz, 1H), 4.29-4.22 (m, 3H), 4.04-3.99 (m, 2H), 3.80 |
| | | | (t, J = 5.0 Hz, 2H), 3.70-3.61 (m, 1H), 3.57-3.45 (m, 2H), 3.27- |
| | | | 3.21 (m, 2H), 2.91-2.81 (m, 1H), 2.79-2.73 (m, 1H), 2.73- |
| | | | 2.67 (m, 2H), 2.59 (br d, J = 4.4 Hz, 1H), 2.55 (s, 2H), 2.35-2.32 |
| | | | (m, 1H), 2.09 (ddd, J = 4.1, 8.3, 12.6 Hz, 2H), 2.02-1.94 (m, |
| | | | 3H), 1.92-1.85 (m, 1H), 1.85-1.78 (m, 3H), 1.76-1.55 (m, 4H), |
| | | | 1.41-1.35 (m, 1H), 1.32 (br d, J = 8.7 Hz, 2H), 1.29-1.23 (m, |
| | | | 2H) |
| 527 | 783 | 784 | δ (DMSO-d6) 10.78 (br s, 1H), 8.38 (d, J = 3.8 Hz, 1H), 7.58 (br |
| | | | d, J = 7.9 Hz, 2H), 7.35 (br d, J = 4.4 Hz, 2H), 7.11 (br d, J = 7.5 |
| | | | Hz, 2H), 6.85-6.78 (m, 1H), 6.54-6.47 (m, 1H), 6.44-6.39 (m, |
| | | | 1H), 5.79 (d, J = 7.7 Hz, 1H), 4.30-4.21 (m, 1H), 3.70-3.58 (m, |
| | | | 1H), 3.48-3.42 (m, 1H), 3.23-3.16 (m, 4H), 2.83 (br s, 4H), |
| | | | 2.76-2.68 (m, 1H), 2.60 (br s, 5H), 2.28-2.18 (m, 1H), 2.12- |
| | | | 1.98 (m, 4H), 1.87-1.78 (m, 5H), 1.74 (br d, J = 11.6 Hz, 2H), |
| | | | 1.64 (br d, J = 3.8 Hz, 4H), 1.57 (br d, J = 4.9 Hz, 2H), 1.51-1.41 |
| | | | (m, 2H), 1.33-1.23 (m, 4H), 1.20 (br s, 2H) |
| 528 | 811 | 812 | δ (DMSO-d6) 10.78 (s, 1H), 8.47 (d, J = 3.5 Hz, 1H), 8.07 (br d, |
| | | | J = 7.5 Hz, 1H), 7.98 (s, 1H), 7.74-7.67 (m, 2H), 7.62-7.58 (m, |
| | | | 1H), 7.55 (ddd, J = 9.1, 6.7, 2.0 Hz, 1H), 7.30 (d, J = 7.7 Hz, 1H), |
| | | | 6.83 (t, J = 9.4 Hz, 1H), 6.53 (s, 1H), 6.51-6.48 (m, 1H), 6.42 |
| | | | (dd, J = 8.7, 2.2 Hz, 1H), 6.36 (dt, J = 6.7, 1.2 Hz, 1H), 5.81 (d, |
| | | | J = 7.6 Hz, 1H), 4.30-4.22 (m, 1H), 4.12 (br d, J = 13.1 Hz, 1H), |
| | | | 3.79 (br d, J = 12.6 Hz, 1H), 3.70-3.60 (m, 1H), 3.30-3.24 (m, |
| | | | 1H), 2.95-2.89 (m, 1H), 2.85 (br s, 4H), 2.78-2.69 (m, 1H), |
| | | | 2.61 (br d, J = 10.4 Hz, 6H), 2.57-2.53 (m, 2H), 2.13-2.06 (m, |
| | | | 1H), 2.00 (br d, J = 10.9 Hz, 2H), 1.93-1.88 (m, 1H), 1.88-1.79 |
| | | | (m, 2H), 1.72 (br d, J = 11.0 Hz, 2H), 1.63-1.56 (m, 1H), 1.47 |
| | | | (br d, J = 9.8 Hz, 2H), 1.41-1.32 (m, 2H), 0.97 (d, J = 6.5 Hz, |
| | | | 1H) |
| 529 | 758 | 759 | δ (DMSO-d6) 10.80 (s, 1H), 8.41 (d, J = 3.7 Hz, 1H), 7.76-7.68 |
| | | | (m, 2H), 7.42 (t, J = 7.7 Hz, 1H), 7.27-7.22 (m, 2H), 6.84 (br t, |
| | | | J = 9.4 Hz, 1H), 6.52 (dd, J = 15.0, 2.1 Hz, 1H), 6.43 (br d, J = |
| | | | 8.4 Hz, 1H), 5.83 (br d, J = 7.2 Hz, 1H), 4.31-4.23 (m, 1H), 4.14 |
| | | | (br d, J = 11.6 Hz, 1H), 3.85-3.76 (m, 1H), 3.66 (br d, J = 7.9 |
| | | | Hz, 1H), 3.32-3.26 (m, 1H), 2.96-2.90 (m, 1H), 2.86 (br s, 4H), |
| | | | 2.79-2.70 (m, 1H), 2.61 (br d, J = 3.3 Hz, 6H), 2.55 (br d, J = |
| | | | 3.8 Hz, 1H), 2.13-2.07 (m, 1H), 2.06-1.98 (m, 3H), 1.96-1.89 |
| | | | (m, 1H), 1.89-1.80 (m, 2H), 1.74 (br d, J = 10.6 Hz, 3H), 1.65- |
| | | | 1.57 (m, 1H), 1.47 (br d, J = 9.4 Hz, 3H), 1.41-1.30 (m, 2H), |
| | | | 1.04-0.99 (m, 2H), 0.75-0.70 (m, 2H) |
| 530 | 817 | 818 | δ (DMSO-d6) 10.84-10.70 (m, 1H), 8.44 (d, J = 3.6 Hz, 1H), |
| | | | 8.00 (s, 1H), 7.88 (br d, J = 5.6 Hz, 1H), 7.63-7.54 (m, 2H), 7.27 |
| | | | (br d, J = 7.5 Hz, 1H), 6.83 (t, J = 9.3 Hz, 1H), 6.51 (dd, J = 14.9, |
| | | | 2.2 Hz, 1H), 6.42 (dd, J = 8.8, 1.7 Hz, 1H), 5.81 (br d, J = 7.6 Hz, |
| | | | 1H), 4.30-4.23 (m, 3H), 4.13 (br d, J = 12.3 Hz, 1H), 4.04-3.98 |
| | | | (m, 2H), 3.83-3.76 (m, 3H), 3.70-3.62 (m, 1H), 3.31-3.25 (m, |
| | | | 1H), 2.92 (br d, J = 11.6 Hz, 1H), 2.85 (br s, 4H), 2.79-2.68 (m, |
| | | | 2H), 2.61 (br d, J = 9.9 Hz, 6H), 2.54 (br s, 1H), 2.09 (td, J = 4.2, |
| | | | 8.7 Hz, 1H), 2.01 (br d, J = 9.5 Hz, 2H), 1.94-1.80 (m, 3H), 1.73 |
| | | | (br d, J = 10.1 Hz, 2H), 1.64-1.56 (m, 1H), 1.49 (br d, J = 5.3 |
| | | | Hz, 3H), 1.37 (br t, J = 11.6 Hz, 2H) |
| 531 | 783 | 784 | δ (DMSO-d6) 10.77 (br s, 1H), 8.38 (d, J = 3.9 Hz, 1H), 7.64- |
| | | | 7.51 (m, 2H), 7.35 (br d, J = 4.4 Hz, 2H), 7.14-7.07 (m, 2H), |
| | | | 6.82 (t, J = 9.3 Hz, 1H), 6.50 (dd, J = 15.1, 2.3 Hz, 1H), 6.41 (dd, |
| | | | J = 8.8, 2.3 Hz, 1H), 5.78 (d, J = 7.6 Hz, 1H), 4.25 (ddd, J = 11.9, |
| | | | 7.5, 4.9 Hz, 1H), 3.69-3.58 (m, 1H), 3.48 (br dd, J = 7.3, 3.8 Hz, |
| | | | 1H), 3.22-3.17 (m, 5H), 3.15 (br s, 2H), 2.87-2.80 (m, 2H), |
| | | | 2.77-2.67 (m, 1H), 2.62-2.57 (m, 1H), 2.55 (br s, 1H), 2.35- |
| | | | 2.26 (m, 1H), 2.20 (br t, J = 10.5 Hz, 2H), 2.12-2.00 (m, 4H), |
| | | | 1.90-1.81 (m, 1H), 1.79-1.71 (m, 5H), 1.64 (br d, J = 4.5 Hz, |
| | | | 5H), 1.60-1.53 (m, 4H), 1.46 (br d, J = 13.3 Hz, 2H), 1.36-1.25 |
| | | | (m, 4H) |
| 532 | 799 | 800 | δ (DMSO-d6) 10.85-10.61 (m, 1H), 8.43 (d, J = 3.6 Hz, 1H), |
| | | | 7.99 (s, 1H), 7.91-7.82 (m, 1H), 7.62-7.55 (m, 3H), 7.19 (br d, |
| | | | J = 7.8 Hz, 1H), 6.82 (t, J = 9.3 Hz, 1H), 6.49 (dd, J = 14.9, 2.3 |
| | | | Hz, 1H), 6.40 (dd, J = 9.0, 1.9 Hz, 1H), 5.78 (d, J = 7.5 Hz, 1H), |
| | | | 4.27-4.17 (m, 3H), 4.03-3.96 (m, 2H), 3.79 (t, J = 4.9 Hz, 2H), |
| | | | 3.70-3.60 (m, 1H), 3.53-3.42 (m, 1H), 3.15 (br d, J = 11.0 Hz, |
| | | | 2H), 2.82 (br d, J = 11.1 Hz, 2H), 2.77-2.66 (m, 1H), 2.57 (br d, |
| | | | J = 4.0 Hz, 1H), 2.54 (br d, J = 1.6 Hz, 1H), 2.35-2.25 (m, 1H), |
| | | | 2.19 (br t, J = 10.6 Hz, 2H), 2.07 (dt, J = 8.3, 4.3 Hz, 2H), 2.03- |
| | | | 1.96 (m, 2H), 1.82 (br dd, J = 12.2, 3.9 Hz, 1H), 1.78-1.66 (m, |
| | | | 6H), 1.61-1.52 (m, 2H), 1.51-1.37 (m, 3H), 1.36-1.23 (m, 4H) |
| 533 | 740 | 741 | δ (DMSO-d6) 10.85-10.67 (m, 1H), 8.39 (d, J = 3.6 Hz, 1H), |
| | | | 7.75-7.65 (m, 2H), 7.60 (br d, J = 7.6 Hz, 1H), 7.40 (t, J = 7.7 |
| | | | Hz, 1H), 7.24 (d, J = 7.8 Hz, 1H), 7.13 (br d, J = 7.9 Hz, 1H), |
| | | | 6.82 (t, J = 9.4 Hz, 1H), 6.49 (dd, J = 15.0, 2.3 Hz, 1H), 6.40 (dd, |
| | | | J = 8.8, 2.1 Hz, 1H), 5.78 (d, J = 7.5 Hz, 1H), 4.29-4.20 (m, 1H), |
| | | | 3.69-3.58 (m, 1H), 3.53-3.42 (m, 1H), 3.15 (br d, J = 11.0 Hz, |
| | | | 2H), 2.82 (br d, J = 11.1 Hz, 2H), 2.77-2.67 (m, 1H), 2.60-2.56 |
| | | | (m, 1H), 2.54 (br s, 1H), 2.29 (br t, J = 11.6 Hz, 1H), 2.19 (br t, |
| | | | J = 10.4 Hz, 2H), 2.12-2.04 (m, 2H), 2.03-1.96 (m, 3H), 1.83 (br |
| | | | dd, J = 12.4, 4.3 Hz, 1H), 1.78-1.67 (m, 6H), 1.60-1.50 (m, |
| | | | 2H), 1.49-1.39 (m, 2H), 1.39-1.19 (m, 5H), 1.03-0.97 (m, 2H), |
| | | | 0.73-0.68 (m, 2H) |
| 534 | 842 | 843 | δ 10.95-10.77 (m, 1H), 8.45 (d, J = 3.6 Hz, 1H), 8.37 (s, 1H), |
| | | | 8.04 (br d, J = 7.6 Hz, 1H), 7.96 (s, 1H), 7.74-7.66 (m, 2H), 7.62- |
| | | | 7.58 (m, 2H), 7.54 (ddd, J = 9.0, 6.8, 2.0 Hz, 1H), 7.43 (s, 1H), |
| | | | 7.21 (br d, J = 7.8 Hz, 1H), 7.03 (d, J = 8.4 Hz, 1H), 6.51 (d, J = |
| | | | 9.2 Hz, 1H), 6.35 (dt, J = 6.8, 1.2 Hz, 1H), 4.32 (dd, J = 9.6, 5.2 |
| | | | Hz, 1H), 3.96 (s, 3H), 3.01 (br d, J = 10.8 Hz, 2H), 2.89 (br d, |
| | | | J = 11.4 Hz, 2H), 2.70-2.56 (m, 3H), 2.44 (br d, J = 3.0 Hz, 4H), |
| | | | 2.38-2.26 (m, 2H), 2.21-2.11 (m, 1H), 2.10-2.00 (m, 3H), 1.99- |
| | | | 1.90 (m, 3H), 1.90-1.83 (m, 2H), 1.82-1.68 (m, 6H), 1.62- |
| | | | 1.48 (m, 4H), 1.39-1.20 (m, 4H) |
| 535 | 827 | 828 | δ (DMSO-d6) 10.87 (br s, 1H), 8.45 (br s, 1H), 8.10-7.91 (m, |
| | | | 2H), 7.76-7.50 (m, 6H), 7.43 (br s, 1H), 7.21 (br d, J = 3.4 Hz, |
| | | | 1H), 7.03 (br d, J = 4.5 Hz, 1H), 6.51 (br d, J = 8.0 Hz, 1H), 6.35 |
| | | | (br s, 1H), 4.32 (br s, 1H), 3.97 (br s, 3H), 3.70-3.58 (m, 1H), |
| | | | 3.53-3.44 (m, 1H), 3.11-2.94 (m, 2H), 2.63 (br dd, J = 6.3, 4.0 |
| | | | Hz, 3H), 2.34 (br dd, J = 6.5, 1.9 Hz, 1H), 2.17 (br d, J = 3.8 Hz, |
| | | | 3H), 1.95 (br d, J = 5.6 Hz, 4H), 1.86-1.67 (m, 9H), 1.57-1.19 |
| | | | (m, 9H), 0.86 (br d, J = 9.5 Hz, 2H) |
| 536 | 799 | 800 | δ 10.80 (s, 1H), 8.45 (d, J = 3.5 Hz, 1H), 8.17 (s, 0.48H), 8.00 (s, |
| | | | 1H), 7.87 (br d, J = 5.1 Hz, 1H), 7.67-7.56 (m, 3H), 7.22 (br d, |
| | | | J = 7.6 Hz, 1H), 6.83 (t, J = 9.4 Hz, 1H), 6.52 (dd, J =15.2, 1.8 |
| | | | Hz, 1H), 6.43 (br d, J = 8.6 Hz, 1H), 5.82 (d, J = 7.6 Hz, 1H), |
| | | | 4.32-4.20 (m, 3H), 4.02 (t, J = 5.0 Hz, 2H), 3.80 (br t, J = 5.0 |
| | | | Hz, 2H), 3.72-3.59 (m, 1H), 3.50 (br dd, J = 5.0, 2.8 Hz, 1H), |
| | | | 2.86 (br s, 4H), 2.79-2.70 (m, 1H), 2.69-2.62 (m, 4H), 2.62- |
| | | | 2.54 (m, 1H), 2.37-2.26 (m, 1H), 2.14-2.06 (m, 1H), 2.05-1.93 |
| | | | (m, 3H), 1.93-1.83 (m, 3H), 1.78 (br t, J = 10.6 Hz, 4H), 1.47- |
| | | | 1.32 (m, 4H), 1.31-1.20 (m, 4H) |
| 537 | 813 | 814 | δ 10.78 (s, 1H), 8.45 (d, J = 3.6 Hz, 1H), 8.11 (br s, 1H), 8.05 (br |
| | | | d, J = 7.2 Hz, 1H), 7.76-7.66 (m, 1H), 7.65-7.52 (m, 2H), 7.22 |
| | | | (br d, J = 7.6 Hz, 1H), 6.82 (t, J = 9.2 Hz, 1H), 6.51 (dd, J = 15.1, |
| | | | 2.2 Hz, 1H), 6.42 (dd, J = 8.8, 2.0 Hz, 1H), 5.80 (d, J = 7.6 Hz, |
| | | | 1H), 5.07-4.93 (m, 1H), 4.42-4.18 (m, 2H), 3.72-3.52 (m, 4H), |
| | | | 3.45-3.38 (m, 1H), 3.28-3.18 (m, 1H), 2.84 (br s, 4H), 2.74 |
| | | | (ddd, J = 17.6, 12.4, 5.4 Hz, 1H), 2.61 (br s, 5H), 2.30-2.20 (m, |
| | | | 1H), 2.09 (td, J = 8.7, 4.2 Hz, 1H), 2.05-1.92 (m, 4H), 1.92- |
| | | | 1.82 (m, 4H), 1.82-1.74 (m, 4H), 1.43-1.30 (m, 4H), 1.29-1.15 |
| | | | (m, 4H) |
| 538 | 799 | 800 | δ (DMSO-d6) 10.79 (s, 1H), 8.47 (d, J = 3.5 Hz, 1H), 8.19 (d, |
| | | | J = 8.3 Hz, 1H), 8.04 (t, J = 8.0 Hz, 1H), 7.92-7.85 (m, 1H), 7.58 |
| | | | (br d, J = 7.6 Hz, 1H), 7.23 (br d, J = 7.8 Hz, 1H), 6.98 (t, J = 8.9 |
| | | | Hz, 1H), 6.48-6.40 (m, 2H), 5.99 (d, J = 7.6 Hz, 1H), 4.30 (s, |
| | | | 2H), 4.03 (s, 3H), 3.70-3.60 (m, 1H), 3.54-3.45 (m, 1H), 2.89 |
| | | | (br d, J = 9.3 Hz, 2H), 2.79-2.64 (m, 3H), 2.33 (br s, 1H), 2.27 |
| | | | (br t, J = 10.2 Hz, 3H), 2.12-2.05 (m, 1H), 2.01-1.95 (m, 3H), |
| | | | 1.92-1.86 (m, 1H), 1.85-1.80 (m, 3H), 1.75 (br d, J = 14.8 Hz, |
| | | | 3H), 1.66-1.55 (m, 4H), 1.42 (br s, 4H), 1.31-1.20 (m, 5H |
| 539 | 799 | 800 | δ (DMSO-d6) 10.83 (s, 1H), 8.47 (d, J = 3.4 Hz, 1H), 8.31 (s, |
| | | | 1H), 8.01-7.97 (m, 2H), 7.87-7.80 (m, 1H), 7.60 (br d, J = 7.6 |
| | | | Hz, 1H), 7.27-7.20 (m, 1H), 7.01-6.94 (m, 1H), 6.48-6.39 (m, |
| | | | 2H), 6.01 (d, J = 7.7 Hz, 1H), 4.38 (t, J = 5.3 Hz, 2H), 4.35-4.27 |
| | | | (m, 1H), 4.00 (t, J = 6.2 Hz, 2H), 3.67-3.63 (m, 1H), 2.91 (br d, |
| | | | J = 10.4 Hz, 2H), 2.74 (dt, J =11.9, 6.2 Hz, 2H), 2.61-2.58 (m, |
| | | | 1H), 2.35-2.27 (m, 4H), 2.17-2.08 (m, 3H), 2.02-1.95 (m, 3H), |
| | | | 1.85-1.73 (m, 7H), 1.68-1.58 (m, 4H), 1.39-1.23 (m, 8H) |
| 540 | 832 | 833 | δ 10.57 (s, 1H), 8.47 (d, J = 3.6 Hz, 1H), 8.13 (s, 0.2H), 8.05 (br |
| | | | s, 1H), 7.97 (s, 1H), 7.74-7.67 (m, 2H), 7.62-7.51 (m, 3H), 7.40 |
| | | | (br d, J = 10.2 Hz, 1H), 7.30 (br d, J = 7.8 Hz, 1H), 6.55-6.48 |
| | | | (m, 1H), 6.35 (dt, J = 6.8, 1.2 Hz, 1H), 4.45-4.36 (m, 1H), 4.01 |
| | | | (s, 4H), 3.90 (t, J = 6.6 Hz, 2H), 3.71-3.58 (m, 1H), 3.09-2.96 |
| | | | (m, 2H), 2.75 (t, J = 6.6 Hz, 2H), 2.61-2.51 (m, 4H), 2.08-1.89 |
| | | | (m, 6H), 1.87-1.65 (m, 6H), 1.55-1.20 (m, 6H), 1.19-0.82 (m, |
| | | | 3H) |
| 541 | 783 | 784 | δ 10.78 (s, 1H), 8.37 (d, J = 3.9 Hz, 1H), 7.72-7.46 (m, 2H), 7.33 |
| | | | (br d, J = 5.6 Hz, 2H), 7.19-7.04 (m, 2H), 6.81 (t, J = 9.3 Hz, |
| | | | 1H), 6.58-6.33 (m, 2H), 5.81 (br d, J = 7.6 Hz, 1H), 4.34-4.16 |
| | | | (m, 1H), 3.68-3.44 (m, 2H), 3.23-3.13 (m, 4H), 2.96-2.78 (m, |
| | | | 4H), 2.78-2.64 (m, 4H), 2.56 (td, J = 3.9, 17.4 Hz, 1H), 2.42- |
| | | | 2.28 (m, 1H), 2.13-1.72 (m, 10H), 1.69-1.49 (m, 6H), 1.47- |
| | | | 1.06 (m, 10H) |
| 542 | 799 | 800 | δ 10.77 (s, 1H), 9.20-8.87 (m, 1H), 8.43 (d, J = 3.6 Hz, 1H), 7.94 |
| | | | (s, 1H), 7.90-7.80 (m, 2H), 7.63-7.48 (m, 2H), 7.18 (br d, J = |
| | | | 7.9 Hz, 1H), 6.85 (t, J = 9.4 Hz, 1H), 6.57-6.36 (m, 2H), 5.85 |
| | | | (br d, J = 7.2 Hz, 1H), 4.36 (t, J = 5.3 Hz, 2H), 4.31-4.19 (m, |
| | | | 1H), 3.71 (t, J = 6.0 Hz, 2H), 3.66 (br s, 1H), 3.60-3.44 (m, 3H), |
| | | | 3.24 (br d, J = 11.4 Hz, 3H), 3.04-2.88 (m, 2H), 2.80-2.52 (m, |
| | | | 5H), 2.43-2.28 (m, 1H), 2.18-2.04 (m, 5H), 2.02-1.69 (m, |
| | | | 10H), 1.42-1.21 (m, 4H) |
| 543 | 799 | 800 | δ 10.78 (br s, 1H), 8.43 (d, J = 3.5 Hz, 1H), 7.95 (s, 1H), 7.84 (br |
| | | | d, J = 6.1 Hz, 1H), 7.66-7.49 (m, 3H), 7.19 (br d, J = 7.7 Hz, |
| | | | 1H), 6.83 (t, J = 9.4 Hz, 1H), 6.55-6.46 (m, 1H), 6.41 (br d, J = |
| | | | 8.6 Hz, 1H), 5.79 (d, J = 7.5 Hz, 1H), 4.38 (t, J = 5.3 Hz, 2H), |
| | | | 4.25 (ddd, J = 4.8, 7.2, 11.6 Hz, 1H), 3.72 (t, J = 6.0 Hz, 2H), 3.65 |
| | | | (br d, J = 6.9 Hz, 1H), 3.56-3.45 (m, 1H), 3.16 (br d, J = 10.7 |
| | | | Hz, 2H), 2.90 (br d, J = 9.7 Hz, 2H), 2.80-2.66 (m, 1H), 2.62- |
| | | | 2.53 (m, 3H), 2.37-2.25 (m, 1H), 2.22-1.92 (m, 8H), 1.90-1.70 |
| | | | (m, 5H), 1.68-1.45 (m, 6H), 1.40-1.20 (m, 4H) |
| 544 | 793 | 794 | δ 11.06-10.54 (m, 1H), 8.42 (d, J = 3.5 Hz, 1H), 8.31 (s, 1H), |
| | | | 8.21 (s, 1H), 7.95-7.81 (m, 2H), 7.76-7.64 (m, 1H), 7.64-7.50 |
| | | | (m, 2H), 7.43 (br d, J = 5.3 Hz, 1H), 7.15 (br d, J = 7.8 Hz, 1H), |
| | | | 6.81 (t, J = 9.4 Hz, 1H), 6.54-6.45 (m, 1H), 6.41 (br d, J = 8.8 |
| | | | Hz, 1H), 6.33 (t, J = 6.6 Hz, 1H), 5.80 (td, J = 5.5, 11.0 Hz, 1H), |
| | | | 4.25 (br dd, J = 4.5, 7.2 Hz, 1H), 3.72-3.57 (m, 2H), 2.83 (br s, |
| | | | 3H), 2.78-2.68 (m, 1H), 2.60 (br s, 4H), 2.23 (br d, J = 8.9 Hz, |
| | | | 1H), 2.09 (dt, J = 4.5, 8.6 Hz, 1H), 2.03-1.68 (m, 9H), 1.49- |
| | | | 1.09 (m, 11H), 0.85 (t, J = 7.3 Hz, 1H) |
| 545 | 799 | 800 | δ 10.79 (s, 1H), 8.46 (d, J = 3.3 Hz, 1H), 8.24-8.13 (m, 1H), 8.04 |
| | | | (t, J = 7.9 Hz, 1H), 7.96-7.84 (m, 1H), 7.59 (br d, J = 8.2 Hz, |
| | | | 1H), 7.23 (br d, J = 7.8 Hz, 1H), 7.03-6.89 (m, 1H), 6.53-6.36 |
| | | | (m, 2H), 6.02 (br d, J = 7.5 Hz, 1H), 4.38-4.23 (m, 3H), 4.02 (s, |
| | | | 4H), 3.71-3.60 (m, 1H), 3.48 (br s, 1H), 3.13-2.96 (m, 2H), |
| | | | 2.83-2.55 (m, 4H), 2.14-1.94 (m, 4H), 1.92-1.58 (m, 12H), |
| | | | 1.55-1.07 (m, 9H) |
| 546 | 797 | 798 | δ 10.78 (s, 1H), 8.44 (d, J = 3.5 Hz, 1H), 8.07-7.73 (m, 3H), 7.55 |
| | | | (br d, J = 7.9 Hz, 1H), 7.20 (br d, J = 7.8 Hz, 1H), 6.98 (t, J = 8.8 |
| | | | Hz, 1H), 6.52-6.32 (m, 2H), 5.98 (d, J = 7.8 Hz, 1H), 4.29 (ddd, |
| | | | J = 4.9, 7.3, 11.8 Hz, 1H), 3.93 (t, J = 5.6 Hz, 2H), 3.65 (br dd, |
| | | | J = 2.6, 3.8 Hz, 1H), 3.53-3.37 (m, 1H), 2.88 (br d, J = 10.4 Hz, |
| | | | 2H), 2.81-2.66 (m, 1H), 2.63-2.52 (m, 3H), 2.35-2.17 (m, 3H), |
| | | | 2.13-1.95 (m, 4H), 1.93-1.68 (m, 11H), 1.67-1.53 (m, 4H), |
| | | | 1.53-1.18 (m, 7H), 1.18-0.97 (m, 2H) |
| 547 | 798 | 799 | δ 10.78 (s, 1H), 8.43 (d, J = 3.6 Hz, 1H), 7.99 (s, 1H), 7.87 (br d, |
| | | | J = 6.0 Hz, 1H), 7.67-7.42 (m, 3H), 7.18 (br d, J = 7.9 Hz, 1H), |
| | | | 7.05-6.85 (m, 1H), 6.55-6.33 (m, 2H), 5.98 (d, J = 7.6 Hz, 1H), |
| | | | 4.37-4.26 (m, 1H), 4.23 (s, 2H), 4.05-3.94 (m, 2H), 3.78 (t, J = |
| | | | 5.0 Hz, 2H), 3.71-3.56 (m, 1H), 3.48-3.37 (m, 1H), 2.89 (br d, |
| | | | J = 10.3 Hz, 2H), 2.80-2.64 (m, 1H), 2.62-2.52 (m, 2H), 2.25 |
| | | | (br t, J = 10.0 Hz, 3H), 2.14-2.02 (m, 2H), 1.99 (br d, J = 11.9 |
| | | | Hz, 2H), 1.86-1.67 (m, 7H), 1.65-1.52 (m, 4H), 1.44 (br d, J = |
| | | | 12.3 Hz, 2H), 1.28 (br t, J = 12.2 Hz, 4H), 1.21-1.08 (m, 2H) |
| 548 | 770 | 771 | δ 10.79 (s, 1H), 8.74 (d, J = 5.3 Hz, 1H), 8.51 (d, J = 3.4 Hz, 1H), |
| | | | 8.16 (s, 0.5H), 8.14 (s, 1H), 7.75 (br d, J = 1.4 Hz, 1H), 7.60 (br |
| | | | d, J = 7.4 Hz, 1H), 7.36-7.29 (m, 1H), 6.98 (t, J = 8.8 Hz, 1H), |
| | | | 6.50-6.40 (m, 2H), 6.02 (d, J = 7.9 Hz, 1H), 5.88 (s, 1H), 4.38- |
| | | | 4.25 (m, 1H), 3.72-3.60 (m, 1H), 3.10-3.01 (m, 2H), 2.80-2.65 |
| | | | (m, 4H), 2.39-2.21 (m, 4H), 2.12-1.90 (m, 6H), 1.90-1.81 (m, |
| | | | 6H), 1.77-1.64 (m, 6H), 1.57-1.08 (m, 10H) |
| 549 | 797 | 798 | δ 10.81 (s, 1H), 8.91 (br s, 1H), 8.61 (d, J = 5.3 Hz, 1H), 8.52 (d, |
| | | | J = 3.4 Hz, 1H), 8.29 (br s, 1H), 7.75-7.62 (m, 2H), 7.32 (br d, |
| | | | J = 6.1 Hz, 1H), 7.01-6.80 (m, 1H), 6.59-6.36 (m, 2H), 4.32 |
| | | | (dd, J = 4.8, 11.5 Hz, 1H), 3.90 (br t, J = 5.7 Hz, 2H), 3.78-3.57 |
| | | | (m, 2H), 3.35-3.06 (m, 4H), 2.95 (dt, J = 5.3, 10.1 Hz, 1H), 2.81- |
| | | | 2.64 (m, 1H), 2.63-2.53 (m, 2H), 2.21-1.95 (m, 7H), 1.95- |
| | | | 1.81 (m, 11H), 1.74 (br d, J = 12.3 Hz, 2H), 1.65-1.10 (m, 9H) |
| 550 | 799 | 800 | δ 10.78 (s, 1H), 8.58 (d, J = 5.3 Hz, 1H), 8.51 (d, J = 3.3 Hz, 1H), |
| | | | 8.39 (s, 1H), 7.67 (br d, J = 1.4 Hz, 1H), 7.55 (br d, J = 8.0 Hz, |
| | | | 1H), 7.31 (br d, J = 7.6 Hz, 1H), 7.02-6.93 (m, 1H), 6.48-6.37 |
| | | | (m, 2H), 5.98 (d, J = 7.8 Hz, 1H), 4.37 (t, J = 5.3 Hz, 2H), 4.33- |
| | | | 4.25 (m, 1H), 3.97 (t, J = 6.1 Hz, 2H), 3.71-3.59 (m, 1H), 3.49- |
| | | | 3.39 (m, 1H), 2.88 (br d, J = 10.1 Hz, 2H), 2.73 (s, 1H), 2.56 (br |
| | | | dd, J = 3.8, 13.5 Hz, 1H), 2.35-2.19 (m, 3H), 2.17-2.12 (m, |
| | | | 2H), 2.10-2.04 (m, 2H), 2.01-1.92 (m, 2H), 1.88-1.83 (m, 1H), |
| | | | 1.82-1.77 (m, 3H), 1.75 (br d, J = 1.9 Hz, 2H), 1.70 (br s, 1H), |
| | | | 1.67-1.53 (m, 5H), 1.49-1.39 (m, 2H), 1.28 (br t, J = 11.1 Hz, |
| | | | 4H), 1.19-1.05 (m, 2H) |
| 551 | 806 | 807 | δ 10.78 (br s, 1H), 8.45 (d, J = 3.6 Hz, 1H), 8.04 (br d, J = 7.5 Hz, |
| | | | 1H), 7.96 (s, 1H), 7.73-7.63 (m, 1H), 7.62-7.49 (m, 3H), 7.42 |
| | | | (dd, J = 2.4, 9.4 Hz, 1H), 7.20 (br d, J = 7.8 Hz, 1H), 6.98 (br t, |
| | | | J = 8.8 Hz, 1H), 6.53-6.28 (m, 3H), 5.98 (br d, J = 7.6 Hz, 1H), |
| | | | 4.37-4.16 (m, 1H), 3.64 (br d, J = 6.1 Hz, 2H), 3.49-3.40 (m, |
| | | | 2H), 2.89 (br d, J = 10.3 Hz, 2H), 2.81-2.65 (m, 2H), 2.61-2.54 |
| | | | (m, 2H), 2.25 (br t, J = 10.2 Hz, 3H), 2.09 (br d, J = 4.1 Hz, 1H), |
| | | | 2.08-2.04 (m, 3H), 2.04 (br s, 1H), 1.98 (br s, 2H), 1.92-1.84 |
| | | | (m, 1H), 1.82-1.72 (m, 4H), 1.70 (br s, 1H), 1.66-1.55 (m, 3H), |
| | | | 1.50-1.38 (m, 2H), 1.35-1.22 (m, 3H), 1.14 (q, J = 11.5 Hz, |
| | | | 2H) |
| 552 | 798 | 799 | δ (DMSO-d6) 10.79 (s, 1H), 8.46-8.42 (m, 1H), 7.99 (s, 1H), |
| | | | 7.90-7.84 (m, 1H), 7.62-7.56 (m, 2H), 7.26 (br d, J = 7.7 Hz, |
| | | | 1H), 7.01-6.93 (m, 1H), 6.51-6.41 (m, 2H), 6.15-5.97 (m, 1H), |
| | | | 4.43-4.27 (m, 2H), 4.24 (s, 2H), 4.02-3.99 (m, 2H), 3.78 (t, J = |
| | | | 5.0 Hz, 2H), 3.70-3.52 (m, 2H), 3.08-2.87 (m, 4H), 2.79-2.66 |
| | | | (m, 2H), 2.59 (br d, J = 4.0 Hz, 1H), 2.57-2.53 (m, 2H), 2.04 (br |
| | | | s, 3H), 2.03-1.96 (m, 3H), 1.95-1.84 (m, 3H), 1.82-1.75 (m, |
| | | | 2H), 1.70 (br d, J = 14.2 Hz, 3H), 1.66-1.57 (m, 2H), 1.52-1.42 |
| | | | (m, 2H), 1.39-1.30 (m, 2H), 1.11-0.82 (m, 2H) |
| 553 | 799 | 800 | δ (DMSO-d6) 10.79 (s, 1H), 8.46-8.41 (m, 1H), 8.15 (s, 1H), |
| | | | 8.02-7.98 (m, 1H), 7.91-7.85 (m, 1H), 7.63-7.55 (m, 2H), 7.27 |
| | | | (br d, J = 7.7 Hz, 1H), 6.83 (t, J = 9.4 Hz, 1H), 6.55-6.48 (m, |
| | | | 1H), 6.42 (dd, J = 8.7, 1.8 Hz, 1H), 5.82 (br d, J = 7.5 Hz, 1H), |
| | | | 4.44-4.35 (m, 1H), 4.29-4.23 (m, 3H), 4.02-3.98 (m, 2H), 3.94 |
| | | | (br d, J = 12.6 Hz, 1H), 3.81-3.77 (m, 2H), 3.63 (br d, J = 11.8 |
| | | | Hz, 2H), 3.00 (br t, J = 12.1 Hz, 2H), 2.86 (br s, 4H), 2.74 (ddd, |
| | | | J = 17.5, 12.0, 5.3 Hz, 2H), 2.59 (br d, J = 4.1 Hz, 1H), 2.54 (br |
| | | | d, J = 4.4 Hz, 2H), 2.20 (br d, J = 6.2 Hz, 2H), 2.14-2.05 (m, |
| | | | 1H), 2.00 (br d, J = 8.9 Hz, 2H), 1.92-1.67 (m, 7H), 1.51-1.28 |
| | | | (m, 4H), 1.07-0.83 (m, 2H) |
| 554 | 739 | 740 | δ (DMSO-d6) 10.79 (br s, 1H), 8.44-8.34 (m, 1H), 7.76-7.67 |
| | | | (m, 2H), 7.41 (t, J = 7.8 Hz, 1H), 7.28-7.17 (m, 2H), 7.03-6.95 |
| | | | (m, 1H), 6.48-6.40 (m, 2H), 6.03-5.97 (m, 1H), 4.43-4.35 (m, |
| | | | 1H), 4.34-4.27 (m, 1H), 3.94 (br d, J = 12.9 Hz, 1H), 3.70-3.60 |
| | | | (m, 1H), 3.05-2.96 (m, 1H), 2.91 (br d, J = 10.5 Hz, 2H), 2.80- |
| | | | 2.69 (m, 1H), 2.59 (br d, J = 4.0 Hz, 1H), 2.58-2.53 (m, 2H), |
| | | | 2.14 (br d, J = 6.6 Hz, 2H), 2.12-2.06 (m, 1H), 2.05-1.97 (m, |
| | | | 3H), 1.97-1.90 (m, 2H), 1.89-1.84 (m, 1H), 1.83-1.76 (m, 2H), |
| | | | 1.72 (br d, J = 12.9 Hz, 4H), 1.63 (br s, 4H), 1.53-1.42 (m, 2H), |
| | | | 1.40-1.30 (m, 2H), 1.05-0.97 (m, 3H), 0.92-0.85 (m, 1H), 0.74- |
| | | | 0.68 (m, 2H) |
| 555 | 740 | 741 | δ (DMSO-d6) 10.80 (s, 1H), 8.41 (d, J = 3.8 Hz, 1H), 8.15 (s, |
| | | | 1H), 7.77-7.67 (m, 2H), 7.42 (t, J = 7.8 Hz, 1H), 7.24 (br dd, |
| | | | J = 7.7, 12.3 Hz, 2H), 6.86 (br t, J = 9.3 Hz, 1H), 6.53 (dd, J = 2.3, |
| | | | 15.1 Hz, 1H), 6.48-6.41 (m, 1H), 5.86 (br d, J = 7.5 Hz, 1H), |
| | | | 4.46-4.36 (m, 1H), 4.28 (ddd, J = 4.8, 7.1, 11.7 Hz, 1H), 4.02- |
| | | | 3.91 (m, 1H), 3.72-3.60 (m, 1H), 3.08-2.84 (m, 6H), 2.74 (ddd, |
| | | | J = 5.3, 12.1, 17.5 Hz, 2H), 2.62-2.54 (m, 4H), 2.13-2.06 (m, |
| | | | 1H), 2.06-1.97 (m, 3H), 1.92-1.68 (m, 7H), 1.56-1.43 (m, 2H), |
| | | | 1.43-1.29 (m, 3H), 1.12-0.87 (m, 5H), 0.75-0.69 (m, 2H) |
| 556 | 782 | 783 | δ (DMSO-d6) 10.81 (s, 1H), 8.39 (d, J = 3.8 Hz, 1H), 8.18 (s, |
| | | | 1H), 7.65 (br d, J = 7.7 Hz, 1H), 7.56 (br s, 1H), 7.35 (br d, J = |
| | | | 5.9 Hz, 2H), 7.17-7.09 (m, 2H), 6.99 (t, J = 8.8 Hz, 1H), 6.51- |
| | | | 6.41 (m, 2H), 6.05 (d, J = 7.7 Hz, 1H), 4.39-4.27 (m, 1H), 3.67- |
| | | | 3.59 (m, 2H), 3.25-3.18 (m, 6H), 3.10 (br d, J = 9.0 Hz, 3H), |
| | | | 2.78-2.67 (m, 2H), 2.61-2.55 (m, 3H), 2.13-2.06 (m, 1H), 2.04- |
| | | | 1.98 (m, 2H), 1.91 (br dd, J = 11.7, 4.6 Hz, 2H), 1.83-1.76 (m, |
| | | | 4H), 1.73 (br s, 3H), 1.65 (br d, J = 4.2 Hz, 5H), 1.58 (br d, J = |
| | | | 5.0 Hz, 2H), 1.46-1.23 (m, 8H) |
| 557 | 740 | 741 | δ (DMSO-d6) 10.85-10.70 (m, 1H), 8.41 (d, J = 3.8 Hz, 1H), |
| | | | 7.76-7.67 (m, 2H), 7.60 (br d, J = 7.7 Hz, 1H), 7.42 (t, J = 7.8 |
| | | | Hz, 1H), 7.25 (d, J = 7.8 Hz, 1H), 7.15 (br d, J = 7.9 Hz, 1H), |
| | | | 6.82 (t, J = 9.4 Hz, 1H), 6.56-6.48 (m, 1H), 6.45-6.40 (m, 1H), |
| | | | 5.80 (d, J = 7.8 Hz, 1H), 4.30-4.22 (m, 1H), 3.71-3.61 (m, 1H), |
| | | | 3.50-3.39 (m, 2H), 2.84 (br s, 4H), 2.79-2.68 (m, 1H), 2.61 (br |
| | | | d, J = 4.0 Hz, 4H), 2.29-2.20 (m, 1H), 2.13-1.97 (m, 5H), 1.90- |
| | | | 1.78 (m, 5H), 1.77-1.70 (m, 2H), 1.53-1.40 (m, 2H), 1.35- |
| | | | 1.23 (m, 4H), 1.21-1.11 (m, 2H), 1.05-0.99 (m, 2H), 0.75-0.70 |
| | | | (m, 2H) |
| 558 | 739 | 740 | δ (DMSO-d6) 10.82-10.72 (m, 1H), 8.42-8.35 (m, 1H), 7.75- |
| | | | 7.65 (m, 2H), 7.62-7.53 (m, 1H), 7.44-7.37 (m, 1H), 7.29-7.21 |
| | | | (m, 1H), 7.13 (br d, J = 7.7 Hz, 1H), 7.02-6.94 (m, 1H), 6.49- |
| | | | 6.38 (m, 2H), 5.98 (br d, J = 7.6 Hz, 1H), 4.35-4.25 (m, 1H), |
| | | | 3.70-3.59 (m, 1H), 3.53-3.46 (m, 1H), 2.94-2.85 (m, 2H), 2.79- |
| | | | 2.66 (m, 2H), 2.36-2.22 (m, 3H), 2.08 (s, 1H), 2.04-1.93 (m, |
| | | | 4H), 1.86-1.71 (m, 7H), 1.69-1.55 (m, 4H), 1.41-1.31 (m, 4H), |
| | | | 1.31-1.17 (m, 5H), 1.05-0.96 (m, 2H), 0.75-0.67 (m, 2H) |
| 559 | 740 | 741 | δ 10.79 (s, 1H), 8.40 (br d, J = 3.4 Hz, 1H), 7.70 (br d, J = 12.0 |
| | | | Hz, 3H), 7.41 (br t, J = 7.6 Hz, 1H), 7.25 (br d, J = 8.0 Hz, 1H), |
| | | | 7.14 (br d, J = 7.4 Hz, 1H), 6.89 (br t, J = 9.0 Hz, 1H), 6.55 (br d, |
| | | | J = 15.0 Hz, 1H), 6.46 (br d, J = 8.4 Hz, 1H), 5.95 (br d, J = 7.2 |
| | | | Hz, 1H), 4.34-4.23 (m, 1H), 3.65 (br d, J = 3.0 Hz, 1H), 3.52- |
| | | | 3.49 (m, 1H), 3.24 (br d, J = 6.0 Hz, 1H), 3.03-2.97 (m, 1H), |
| | | | 2.81-2.67 (m, 2H), 2.63-2.53 (m, 2H), 2.17-1.93 (m, 9H), 1.92- |
| | | | 1.77 (m, 6H), 1.52-1.38 (m, 5H), 1.37-1.22 (m, 5H), 1.01 (br |
| | | | d, J = 7.6 Hz, 2H), 0.72 (br d, J = 4.8 Hz, 2H) |
| 560 | 825 | 826 | δ (DMSO-d6) 10.77 (br s, 1H), 8.42 (d, J = 3.7 Hz, 1H), 7.90- |
| | | | 7.80 (m, 2H), 7.68 (d, J = 7.7 Hz, 1H), 7.55 (t, J = 7.8 Hz, 1H), |
| | | | 7.45 (br d, J = 8.1 Hz, 1H), 7.18 (d, J = 7.7 Hz, 1H), 6.82 (t, J = |
| | | | 9.4 Hz, 1H), 6.51 (dd, J = 15.0, 2.3 Hz, 1H), 6.42 (dd, J = 8.7, 2.1 |
| | | | Hz, 1H), 5.80 (d, J = 7.7 Hz, 1H), 4.26 (ddd, J = 11.6, 7.3, 4.8 Hz, |
| | | | 1H), 3.68-3.60 (m, 3H), 3.57-3.46 (m, 1H), 2.84 (br s, 4H), |
| | | | 2.78-2.69 (m, 1H), 2.62-2.56 (m, 1H), 2.53 (d, J = 2.0 Hz, 2H), |
| | | | 2.47-2.40 (m, 5H), 2.14-2.06 (m, 3H), 1.99-1.85 (m, 8H), 1.83- |
| | | | 1.74 (m, 4H), 1.72-1.55 (m, 3H), 1.47-1.38 (m, 1H), 1.36- |
| | | | 1.20 (m, 4H), 0.99-0.71 (m, 4H) |
| 561 | 827 | 828 | δ (DMSO-d6) 10.78 (s, 1H), 8.43 (d, J = 3.7 Hz, 1H), 7.95 (s, |
| | | | 1H), 7.84 (br d, J = 6.0 Hz, 1H), 7.68 (br d, J = 7.7 Hz, 1H), 7.61- |
| | | | 7.51 (m, 2H), 7.18 (br d, J = 7.8 Hz, 1H), 6.82 (t, J = 9.3 Hz, |
| | | | 1H), 6.51 (dd, J = 15.2, 2.2 Hz, 1H), 6.42 (dd, J = 8.7, 2.1 Hz, |
| | | | 1H), 5.80 (d, J = 7.8 Hz, 1H), 4.37 (t, J = 5.3 Hz, 2H), 4.26 (ddd, |
| | | | J = 11.5, 7.2, 4.8 Hz, 1H), 3.71 (t, J = 6.1 Hz, 2H), 3.67-3.59 (m, |
| | | | 1H), 3.56-3.48 (m, 1H), 2.84 (br s, 4H), 2.76-2.67 (m, 1H), |
| | | | 2.44 (br s, 4H), 2.17-2.07 (m, 5H), 2.00-1.89 (m, 4H), 1.86- |
| | | | 1.73 (m, 5H), 1.71-1.57 (m, 3H), 1.50-1.39 (m, 1H), 1.35-1.20 |
| | | | (m, 4H), 1.00-0.76 (m, 5H) |
| 562 | 827 | 828 | δ (DMSO-d6) 10.79 (s, 1H), 8.44 (d, J = 3.7 Hz, 1H), 8.00 (s, |
| | | | 1H), 7.90-7.83 (m, 1H), 7.72-7.65 (m, 1H), 7.63-7.56 (m, 2H), |
| | | | 7.21 (br d, J = 7.8 Hz, 1H), 6.83 (t, J = 9.4 Hz, 1H), 6.51 (dd, J = |
| | | | 2.0, 15.2 Hz, 1H), 6.42 (dd, J = 1.8, 8.7 Hz, 1H), 5.86-5.76 (m, |
| | | | 1H), 4.30-4.22 (m, 3H), 4.03-3.98 (m, 2H), 3.82-3.77 (m, 2H), |
| | | | 3.69-3.60 (m, 1H), 3.56-3.46 (m, 1H), 2.85 (br s, 4H), 2.79- |
| | | | 2.64 (m, 2H), 2.59 (br d, J = 4.4 Hz, 1H), 2.45 (br d, J = 2.8 Hz, |
| | | | 2H), 2.17-2.05 (m, 3H), 2.00-1.90 (m, 4H), 1.88-1.83 (m, 1H), |
| | | | 1.83-1.75 (m, 4H), 1.70-1.58 (m, 3H), 1.47-1.38 (m, 1H), 1.37- |
| | | | 1.20 (m, 5H), 0.98-0.78 (m, 4H) |
| 563 | 798 | 799 | δ 10.79 (s, 1H), 8.95-8.85 (m, 1H), 8.42 (d, J = 3.6 Hz, 1H), 7.94 |
| | | | (s, 1H), 7.86-7.81 (m, 1H), 7.66 (br d, J = 7.8 Hz, 1H), 7.58- |
| | | | 7.51 (m, 2H), 7.24-7.12 (m, 1H), 6.94 (s, 1H), 6.51-6.44 (m, |
| | | | 2H), 4.36 (t, J = 5.2 Hz, 2H), 4.31 (br dd, J = 11.8, 4.8 Hz, 1H), |
| | | | 3.73-3.68 (m, 4H), 3.50 (br s, 1H), 3.48-3.45 (m, 1H), 3.21- |
| | | | 3.07 (m, 3H), 2.93-2.93 (m, 1H), 3.00-2.88 (m, 1H), 2.64-2.54 |
| | | | (m, 1H), 2.16-2.07 (m, 3H), 2.03 (br d, J = 14.8 Hz, 5H), 1.90 |
| | | | (br d, J = 10 Hz, 7H), 1.77-1.69 (m, 2H), 1.57 (br d, J = 11.0 Hz, |
| | | | 2H), 1.45 (br d, J = 12.2 Hz, 2H), 1.32-1.16 (m, 4H) |
| 564 | 797 | 798 | δ 10.79 (s, 1H), 8.43 (d, J = 3.6 Hz, 1H), 7.90-7.81 (m, 2H), 7.63 |
| | | | (br d, J = 7.2 Hz, 1H), 7.56 (t, J = 7.8 Hz, 1H), 7.49-7.42 (m, |
| | | | 1H), 7.19 (br d, J = 7.6 Hz, 1H), 6.86 (br t, J = 9.2 Hz, 1H), 6.53 |
| | | | (br d, J = 15.2 Hz, 1H), 6.44 (br d, J = 9.0 Hz, 1H), 5.88 (br d, |
| | | | J = 7.6 Hz, 1H), 4.35-4.20 (m, 1H), 3.66 (br t, J = 5.4 Hz, 3H), |
| | | | 3.50-3.43 (m, 1H), 3.07-2.87 (m, 7H), 2.79-2.68 (m, 2H), 2.43 |
| | | | (br t, J = 6.2 Hz, 2H), 2.13-2.05 (m, 2H), 2.00 (br d, J = 13.4 Hz, |
| | | | 4H), 1.92-1.82 (m, 9H), 1.74 (br d, J = 10.4 Hz, 2H), 1.51-1.38 |
| | | | (m, 4H), 1.32-1.18 (m, 4H) |
| 565 | 796 | 797 | δ 10.78 (s, 1H), 8.42 (d, J = 3.6 Hz, 1H), 8.15 (s, 0.1H), 7.87 (s, |
| | | | 2H), 7.59-7.53 (m, 2H), 7.45 (br d, J = 7.6 Hz, 1H), 7.17 (br d, |
| | | | J = 8 Hz, 1H), 6.97 (br t, J = 8.8 Hz, 1H), 6.48-6.41 (m, 2H), |
| | | | 6.00 (br d, J = 7.6 Hz, 1H), 4.30 (br s, 1H), 3.68-3.60 (m, 3H), |
| | | | 3.49-3.40 (m, 1H), 3.07-2.95 (m, 2H), 2.80-2.65 (m, 2H), 2.61- |
| | | | 2.55 (m, 1H), 2.42 (br t, J = 6.4 Hz, 3H), 2.11-1.95 (m, 4H), |
| | | | 1.92-1.77 (m, 10H), 1.76-1.62 (m, 6H), 1.51-1.14 (m, 9H) |
| 566 | 797 | 798 | δ (DMSO-d6) 10.84-10.74 (m, 1H), 8.45 (d, J = 3.5 Hz, 1H), |
| | | | 8.01-7.94 (m, 1H), 7.92-7.88 (m, 1H), 7.85-7.78 (m, 1H), 7.60 |
| | | | (br d, J = 8.0 Hz, 1H), 7.22 (br d, J = 7.5 Hz, 1H), 7.02-6.94 (m, |
| | | | 1H), 6.50-6.39 (m, 2H), 6.01 (br d, J = 7.8 Hz, 1H), 4.31 (ddd, |
| | | | J = 4.5, 7.2, 11.8 Hz, 1H), 3.97-3.88 (m, 2H), 3.72-3.59 (m, |
| | | | 1H), 3.54-3.44 (m, 1H), 3.06-2.90 (m, 2H), 2.79-2.67 (m, 1H), |
| | | | 2.61-2.53 (m, 2H), 2.46-2.36 (m, 2H), 2.13-2.04 (m, 1H), 2.04- |
| | | | 1.91 (m, 4H), 1.91-1.72 (m, 12H), 1.72-1.60 (m, 4H), 1.45- |
| | | | 1.21 (m, 9H) |
| 567 | 793 | 794 | δ (DMSO-d6) 10.79 (br s, 1H), 8.49 (d, J = 3.3 Hz, 1H), 8.22- |
| | | | 8.16 (m, 1H), 8.09 (br d, J = 6.4 Hz, 1H), 7.99-7.93 (m, 2H), |
| | | | 7.61-7.54 (m, 2H), 7.30 (br d, J = 7.6 Hz, 1H), 7.01-6.95 (m, |
| | | | 1H), 6.56 (d, J = 9.1 Hz, 1H), 6.47-6.43 (m, 2H), 6.43-6.39 (m, |
| | | | 1H), 5.99 (d, J = 7.8 Hz, 1H), 4.30 (ddd, J = 11.7, 7.3, 4.8 Hz, |
| | | | 1H), 3.71-3.61 (m, 1H), 3.54-3.45 (m, 1H), 2.88 (br d, J = 10.5 |
| | | | Hz, 2H), 2.79-2.69 (m, 1H), 2.59 (br d, J = 3.9 Hz, 1H), 2.57- |
| | | | 2.54 (m, 1H), 2.27 (br t, J = 9.9 Hz, 3H), 2.12-2.05 (m, 1H), 1.97 |
| | | | (br d, J = 11.3 Hz, 3H), 1.92-1.86 (m, 1H), 1.84-1.76 (m, 5H), |
| | | | 1.73 (br s, 1H), 1.67-1.55 (m, 4H), 1.40-1.32 (m, 4H), 1.31- |
| | | | 1.21 (m, 4H) |
| 568 | 798 | 799 | δ (DMSO-d6) 10.87-10.61 (m, 1H), 8.43 (br d, J = 3.4 Hz, 1H), |
| | | | 7.99 (br s, 1H), 7.86 (br d, J = 4.1 Hz, 1H), 7.58 (br d, J = 5.6 Hz, |
| | | | 3H), 7.18 (br d, J = 7.8 Hz, 1H), 6.98 (br t, J = 8.8 Hz, 1H), 6.44 |
| | | | (br s, 2H), 5.98 (br d, J = 7.4 Hz, 1H), 4.34-4.20 (m, 3H), 4.00 |
| | | | (br t, J = 4.7 Hz, 2H), 3.79 (br t, J = 4.5 Hz, 2H), 3.67-3.59 (m, |
| | | | 1H), 3.51-3.44 (m, 1H), 2.92-2.83 (m, 2H), 2.78-2.70 (m, 1H), |
| | | | 2.60-2.54 (m, 2H), 2.26 (br t, J = 9.7 Hz, 3H), 2.08 (br dd, J = |
| | | | 12.5, 4.3 Hz, 1H), 1.96 (br d, J = 9.5 Hz, 3H), 1.88-1.72 (m, 7H), |
| | | | 1.67-1.53 (m, 4H), 1.41 (br s, 8H) |
| 569 | 770 | 771 | δ (DMSO-d6) 10.88-10.69 (m, 1H), 8.73 (d, J = 5.0 Hz, 1H), |
| | | | 8.50 (d, J = 3.5 Hz, 1H), 8.13 (s, 1H), 7.74 (br d, J = 4.1 Hz, 1H), |
| | | | 7.59 (br d, J = 7.9 Hz, 1H), 7.32 (br d, J = 7.6 Hz, 1H), 7.02-6.96 |
| | | | (m, 1H), 6.46-6.42 (m, 1H), 5.98 (d, J = 7.5 Hz, 1H), 5.94-5.77 |
| | | | (m, 1H), 4.32-4.24 (m, 1H), 3.65 (br d, J = 7.0 Hz, 1H), 3.50 (br |
| | | | dd, J = 6.9, 3.6 Hz, 1H), 2.89 (br d, J = 10.6 Hz, 2H), 2.77-2.67 |
| | | | (m, 1H), 2.60-2.54 (m, 3H), 2.31-2.20 (m, 5H), 2.11-2.05 (m, |
| | | | 1H), 2.02-1.92 (m, 4H), 1.87-1.72 (m, 8H), 1.68-1.55 (m, 4H), |
| | | | 1.45-1.16 (m, 9H) |
| 570 | 799 | 800 | δ (DMSO-d6) 10.84-10.67 (m, 1H), 8.63 (d, J = 5.2 Hz, 1H), |
| | | | 8.51 (d, J = 3.3 Hz, 1H), 7.72 (br d, J = 3.8 Hz, 1H), 7.58 (br d, |
| | | | J = 7.6 Hz, 1H), 7.31 (br d, J = 7.6 Hz, 1H), 6.98 (br t, J = 8.7 Hz, |
| | | | 1H), 6.47-6.40 (m, 2H), 5.97 (br d, J = 7.5 Hz, 1H), 4.30 (s, 3H), |
| | | | 4.02 (s, 4H), 3.64 (br d, J = 6.6 Hz, 1H), 3.54-3.44 (m, 1H), 2.88 |
| | | | (br d, J = 10.5 Hz, 2H), 2.78-2.66 (m, 1H), 2.60-2.53 (m, 2H), |
| | | | 2.26 (br t, J = 10.0 Hz, 3H), 2.08 (dt, J = 8.3, 4.1 Hz, 1H), 2.02- |
| | | | 1.93 (m, 3H), 1.78 (dt, J = 14.5, 13.6 Hz, 8H), 1.66-1.54 (m, |
| | | | 4H), 1.39-1.20 (m, 8H) |
| 571 | 797 | 798 | δ (DMSO-d6) 10.78 (s, 1H), 8.60 (d, J = 5.2 Hz, 1H), 8.50 (d, |
| | | | J = 3.2 Hz, 1H), 8.28 (br s, 1H), 8.20 (br s, 1H), 7.67 (br d, J = 5.1 |
| | | | Hz, 1H), 7.60 (br d, J = 7.7 Hz, 1H), 7.30 (br d, J = 7.7 Hz, 1H), |
| | | | 7.01-6.94 (m, 1H), 6.48-6.40 (m, 2H), 6.00 (br d, J = 7.6 Hz, |
| | | | 1H), 4.30 (br s, 1H), 3.89 (br t, J = 5.5 Hz, 2H), 3.68-3.59 (m, |
| | | | 1H), 3.53-3.46 (m, 1H), 3.01 (br d, J = 10.5 Hz, 2H), 2.80-2.68 |
| | | | (m, 1H), 2.67-2.57 (m, 2H), 2.56-2.51 (m, 2H), 2.45 (br s, 2H), |
| | | | 2.07 (br dd, J = 8.4, 4.0 Hz, 1H), 1.95 (br d, J = 11.3 Hz, 3H), |
| | | | 1.92-1.82 (m, 8H), 1.78 (br s, 4H), 1.64 (br s, 4H), 1.41-1.23 |
| | | | (m, 8H) |
| 572 | 799 | 800 | δ (DMSO-d6) 10.81-10.73 (m, 1H), 8.59 (d, J = 5.1 Hz, 1H), |
| | | | 8.51 (d, J = 3.5 Hz, 1H), 8.47-8.36 (m, 1H), 7.68 (br d, J = 4.8 |
| | | | Hz, 1H), 7.60-7.55 (m, 1H), 7.29 (br d, J = 7.5 Hz, 1H), 7.03- |
| | | | 6.96 (m, 1H), 6.49-6.41 (m, 2H), 5.97 (br d, J = 7.0 Hz, 1H), |
| | | | 4.39 (t, J = 5.4 Hz, 2H), 4.34-4.25 (m, 1H), 3.98 (t, J = 6.2 Hz, |
| | | | 2H), 3.71-3.61 (m, 1H), 3.52-3.45 (m, 1H), 2.89 (br d, J = 10.4 |
| | | | Hz, 2H), 2.79-2.68 (m, 1H), 2.60 (br d, J = 4.2 Hz, 1H), 2.27 (br |
| | | | t, J = 10.1 Hz, 3H), 2.18-2.06 (m, 4H), 2.01-1.95 (m, 3H), 1.85- |
| | | | 1.72 (m, 7H), 1.66-1.56 (m, 4H), 1.39-1.22 (m, 8H) |
| 573 | 793 | 794 | δ (DMSO-d6) 10.83-10.75 (m, 1H), 8.85-8.76 (m, 1H), 8.59- |
| | | | 8.53 (m, 1H), 8.33-8.28 (m, 1H), 7.97 (br dd, J = 12.4, 5.7 Hz, |
| | | | 2H), 7.62-7.54 (m, 2H), 7.38 (br d, J = 7.9 Hz, 1H), 7.02-6.95 |
| | | | (m, 1H), 6.56 (d, J = 9.3 Hz, 1H), 6.47-6.39 (m, 3H), 6.01-5.96 |
| | | | (m, 1H), 4.34-4.27 (m, 1H), 3.70-3.63 (m, 1H), 3.53-3.45 (m, |
| | | | 1H), 2.89 (br d, J = 9.9 Hz, 2H), 2.79-2.68 (m, 1H), 2.59 (br d, |
| | | | J = 4.0 Hz, 1H), 2.57-2.54 (m, 1H), 2.34-2.24 (m, 3H), 2.12- |
| | | | 2.04 (m, 1H), 1.97 (br d, J = 11.1 Hz, 3H), 1.86-1.74 (m, 7H), |
| | | | 1.66-1.54 (m, 4H), 1.42-1.32 (m, 4H), 1.30-1.21 (m, 4H) |
| 574 | 806 | 807 | δ (DMSO-d6) 10.79 (s, 1H), 8.46 (d, J = 3.6 Hz, 1H), 8.03 (br d, |
| | | | J = 7.8 Hz, 1H), 7.97 (s, 1H), 7.72-7.64 (m, 1H), 7.61-7.56 (m, |
| | | | 2H), 7.53 (s, 1H), 7.43 (dd, J = 9.4, 2.5 Hz, 1H), 7.22 (br d, J = |
| | | | 7.9 Hz, 1H), 7.04-6.93 (m, 1H), 6.49-6.41 (m, 3H), 5.99 (d, |
| | | | J = 7.6 Hz, 1H), 4.35-4.25 (m, 1H), 3.69-3.61 (m, 1H), 3.54- |
| | | | 3.44 (m, 1H), 2.90 (br d, J = 10.6 Hz, 2H), 2.80-2.69 (m, 1H), |
| | | | 2.61-2.53 (m, 2H), 2.28 (br t, J = 9.9 Hz, 3H), 2.07 (s, 3H), 1.97 |
| | | | (br d, J = 11.6 Hz, 3H), 1.85-1.73 (m, 7H), 1.67-1.56 (m, 4H), |
| | | | 1.40-1.30 (m, 4H), 1.29-1.21 (m, 4H), 0.97 (d, J = 6.5 Hz, 1H) |
| 575 | 805 | 806 | δ 10.76 (s, 1H), 8.45 (d, J = 3.7 Hz, 1H), 8.27-8.18 (m, 0.8 H), |
| | | | 8.04 (br d, J = 7.6 Hz, 1H), 7.96 (br s, 1H), 7.74-7.66 (m, 2H), |
| | | | 7.61-7.52 (m, 3H), 7.21 (br d, J = 7.8 Hz, 1H), 6.81 (t, J = 9.4 |
| | | | Hz, 1H), 6.54-6.45 (m, 2H), 6.40 (dd, J = 8.8, 2.0 Hz, 1H), 6.35 |
| | | | (dt, J = 6.8, 1.2 Hz, 1H), 5.77 (br d, J = 7.6 Hz, 1H), 4.30-4.18 |
| | | | (m, 1H), 3.72-3.57 (m, 2H), 3.50-3.42 (m, 2H), 3.18 (br s, 2H), |
| | | | 3.07 (br s, 3H), 2.84-2.75 (m, 1H), 2.74-2.68 (m, 1H), 2.60- |
| | | | 2.55 (m, 1H), 2.53 (br s, 1H), 2.15 (br t, J = 9.0 Hz, 3H), 2.07 (dt, |
| | | | J = 8.8, 4.6 Hz, 2H), 1.95 (br d, J = 9.4 Hz, 2H), 1.89-1.81 (m, |
| | | | 1H), 1.77 (br d, J = 10.2 Hz, 2H), 1.67 (br d, J = 9.8 Hz, 2H), 1.37- |
| | | | 1.16 (m, 7H) |
| 576 | 798 | 799 | δ (DMSO-d6) 10.77 (br s, 1H), 8.43 (d, J = 3.7 Hz, 1H), 7.96 (s, |
| | | | 1H), 7.84 (br d, J = 5.6 Hz, 1H), 7.63-7.47 (m, 3H), 7.22-7.10 |
| | | | (m, 1H), 6.99 (br t, J = 8.8 Hz, 1H), 6.50-6.38 (m, 2H), 5.97 (br |
| | | | d, J = 7.6 Hz, 1H), 4.38 (t, J = 5.3 Hz, 2H), 4.35-4.27 (m, 1H), |
| | | | 3.72 (br t, J = 6.0 Hz, 2H), 3.68-3.59 (m, 1H), 3.54-3.44 (m, |
| | | | 1H), 2.89 (br d, J = 10.0 Hz, 2H), 2.79-2.67 (m, 1H), 2.58 (br |
| | | | dd, J = 13.7, 3.8 Hz, 2H), 2.27 (br t, J = 10.3 Hz, 3H), 2.17-2.07 |
| | | | (m, 3H), 1.97 (br d, J = 11.4 Hz, 3H), 1.88-1.72 (m, 7H), 1.68- |
| | | | 1.55 (m, 4H), 1.41-1.19 (m, 8H) |
| 577 | 793 | 794 | δ 11.91 (br s, 1H), 10.83 (s, 1H), 8.64-8.18 (m, 2H), 8.00-7.89 |
| | | | (m, 2H), 7.76 (dd, J = 1.9, 6.9 Hz, 1H), 7.70-7.56 (m, 2H), 7.49 |
| | | | (br d, J = 4.9 Hz, 1H), 7.21 (br d, J = 7.8 Hz, 1H), 6.88 (t, J = 9.3 |
| | | | Hz, 1H), 6.55 (dd, J = 1.9, 15.1 Hz, 1H), 6.46 (br d, J = 8.8 Hz, |
| | | | 1H), 6.39 (t, J = 6.6 Hz, 1H), 5.84 (br d, J = 7.6 Hz, 1H), 4.37- |
| | | | 4.23 (m, 1H), 3.70 (br d, J = 6.9 Hz, 1H), 3.58-3.52 (m, 1H), |
| | | | 3.22 (br d, J = 10.8 Hz, 2H), 3.04-2.91 (m, 2H), 2.76 (td, J = 5.5, |
| | | | 17.4 Hz, 1H), 2.64 (br d, J = 4.4 Hz, 2H), 2.42-2.32 (m, 1H), |
| | | | 2.14 (br dd, J = 4.7, 13.3 Hz, 3H), 2.03 (br d, J = 10.3 Hz, 2H), |
| | | | 1.97-1.78 (m, 5H), 1.76-1.52 (m, 6H), 1.48-1.19 (m, 6H) |
| 578 | 814 | 815 | δ 10.78 (s, 1H), 8.44 (d, J = 3.5 Hz, 1H), 8.00 (s, 1H), 7.88 (br d, |
| | | | J = 5.8 Hz, 1H), 7.68-7.51 (m, 2H), 7.26 (br d, J = 7.6 Hz, 1H), |
| | | | 6.98 (t, J = 8.8 Hz, 1H), 6.52-6.38 (m, 2H), 5.99 (d, J = 7.8 Hz, |
| | | | 1H), 4.37-4.27 (m, 1H), 4.24 (s, 2H), 4.21 (s, 1H), 4.05 (br d, |
| | | | J = 12.8 Hz, 1H), 4.02-3.95 (m, 2H), 3.79 (t, J = 4.9 Hz, 2H), 3.67 |
| | | | (br d, J = 12.4 Hz, 2H), 3.42-3.34 (m, 1H), 3.07-2.88 (m, 3H), |
| | | | 2.73 (dt, J = 6.0, 11.8 Hz, 1H), 2.63-2.52 (m, 3H), 2.28 (s, 2H), |
| | | | 2.22 (br t, J = 11.1 Hz, 2H), 2.08 (dt, J = 4.4, 8.6 Hz, 1H), 2.00 |
| | | | (br d, J = 9.4 Hz, 2H), 1.93-1.80 (m, 1H), 1.78-1.56 (m, 6H), |
| | | | 1.55-1.26 (m, 8H) |
| 579 | 812 | 813 | δ 10.78 (s, 1H), 8.42 (d, J = 3.6 Hz, 1H), 7.97-7.74 (m, 2H), 7.55 |
| | | | (t, J = 7.9 Hz, 1H), 7.45 (br d, J = 7.9 Hz, 1H), 7.24 (br d, J = 7.5 |
| | | | Hz, 1H), 6.97 (t, J = 8.8 Hz, 1H), 6.52-6.38 (m, 2H), 5.98 (br d, |
| | | | J = 7.8 Hz, 1H), 4.37-4.27 (m, 1H), 4.20 (br s, 1H), 4.04 (br d, |
| | | | J = 12.5 Hz, 1H), 3.64 (br t, J = 5.4 Hz, 4H), 3.04-2.89 (m, 3H), |
| | | | 2.77-2.69 (m, 2H), 2.60-2.53 (m, 3H), 2.41 (br t, J = 6.3 Hz, |
| | | | 2H), 2.35-2.18 (m, 4H), 2.11-1.97 (m, 3H), 1.91-1.80 (m, 5H), |
| | | | 1.73-1.32 (m, 14H) |
| 580 | 814 | 815 | δ 10.78 (s, 1H), 8.44 (d, J = 3.6 Hz, 1H), 7.96 (s, 1H), 7.85 (br d, |
| | | | J = 6.5 Hz, 1H), 7.61-7.46 (m, 2H), 7.25 (br d, J = 7.6 Hz, 1H), |
| | | | 6.98 (t, J = 8.8 Hz, 1H), 6.50-6.36 (m, 2H), 5.99 (d, J = 7.8 Hz, |
| | | | 1H), 4.41-4.35 (m, 2H), 4.35-4.26 (m, 1H), 4.21 (br s, 1H), |
| | | | 4.05 (br d, J = 12.5 Hz, 1H), 3.75-3.60 (m, 4H), 3.06-2.90 (m, |
| | | | 3H), 2.80-2.67 (m, 1H), 2.63-2.53 (m, 3H), 2.32-2.17 (m, 4H), |
| | | | 2.16-2.05 (m, 3H), 2.00 (br d, J = 9.5 Hz, 2H), 1.86 (dq, J = 4.6, |
| | | | 12.2 Hz, 1H), 1.79-1.22 (m, 15H), 1.04 (d, J = 6.1 Hz, 1H). |
| 581 | 808 | 809 | δ 12.01-11.62 (m, 1H), 10.78 (s, 1H), 8.42 (d, J = 3.5 Hz, 1H), |
| | | | 8.33 (br s, 1H), 8.04-7.84 (m, 2H), 7.81-7.66 (m, 1H), 7.55 (t, |
| | | | J = 7.8 Hz, 1H), 7.47-7.40 (m, 1H), 7.21 (br d, J = 7.5 Hz, 1H), |
| | | | 6.97 (t, J = 8.8 Hz, 1H), 6.50-6.39 (m, 2H), 6.36-6.27 (m, 1H), |
| | | | 5.98 (d, J = 7.6 Hz, 1H), 4.36-4.14 (m, 2H), 4.04 (br d, J = 12.6 |
| | | | Hz, 1H), 3.73-3.57 (m, 2H), 3.08-2.89 (m, 3H), 2.80-2.67 (m, |
| | | | 1H), 2.64-2.52 (m, 3H), 2.37-2.14 (m, 4H), 2.12-1.93 (m, 3H), |
| | | | 1.85 (dq, J = 4.6, 12.2 Hz, 1H), 1.75-1.28 (m, 14H), 0.97 (br d, |
| | | | J = 6.3 Hz, 1H) |
| 582 | 813 | 814 | δ 10.78 (s, 1H), 8.43 (d, J = 3.8 Hz, 1H), 7.92-7.79 (m, 2H), 7.61- |
| | | | 7.52 (m, 1H), 7.45 (br d, J = 8.5 Hz, 1H), 7.25 (br d, J = 7.1 Hz, |
| | | | 1H), 6.83 (br t, J = 9.1 Hz, 1H), 6.56-6.38 (m, 2H), 5.88-5.75 |
| | | | (m, 1H), 4.37-4.19 (m, 1H), 4.11-3.96 (m, 1H), 3.75-3.54 (m, |
| | | | 4H), 3.06-2.91 (m, 2H), 2.84 (br dd, J = 2.1, 4.4 Hz, 4H), 2.72- |
| | | | 2.56 (m, 6H), 2.42 (br t, J = 6.3 Hz, 3H), 2.37-2.24 (m, 2H), 2.15- |
| | | | 1.95 (m, 3H), 1.95-1.80 (m, 5H), 1.76-1.63 (m, 2H), 1.63- |
| | | | 1.19 (m, 9H). |
| 583 | 815 | 816 | δ 10.77 (s, 1H), 8.43 (d, J = 3.6 Hz, 1H), 7.95 (s, 1H), 7.84 (br d, |
| | | | J = 6.6 Hz, 1H), 7.59-7.50 (m, 2H), 7.24 (br d, J = 7.6 Hz, 1H), |
| | | | 6.81 (t, J = 9.4 Hz, 1H), 6.50 (dd, J = 2.4, 14.9 Hz, 1H), 6.41 (dd, |
| | | | J = 2.3, 8.8 Hz, 1H), 5.79 (d, J = 7.6 Hz, 1H), 4.36 (t, J = 5.4 Hz, |
| | | | 2H), 4.26 (s, 2H), 4.05-3.98 (m, 1H), 3.71 (br t, J = 6.1 Hz, 4H), |
| | | | 3.01-2.93 (m, 1H), 2.83 (br s, 4H), 2.78-2.73 (m, 1H), 2.64 (br |
| | | | s, 3H), 2.58-2.58 (m, 2H), 2.60-2.56 (m, 1H), 2.54 (br s, 1H), |
| | | | 2.30 (s, 2H), 2.16-2.09 (m, 3H), 2.01 (br s, 2H), 1.84 (br dd, J = |
| | | | 4.0, 12.4 Hz, 1H), 1.70 (br d, J = 11.8 Hz, 2H), 1.55-1.42 (m, |
| | | | 5H), 1.35 (br s, 3H) |
| 584 | 809 | 810 | δ 11.93-11.80 (m, 1H), 10.77 (s, 1H), 8.42 (d, J = 3.5 Hz, 1H), |
| | | | 8.33 (br s, 1H), 8.03-7.74 (m, 3H), 7.70 (dd, J = 2.1, 6.9 Hz, |
| | | | 1H), 7.55 (t, J = 7.8 Hz, 1H), 7.42 (br d, J = 5.8 Hz, 1H), 7.21 (br |
| | | | d, J = 7.3 Hz, 1H), 6.82 (br s, 1H), 6.50 (br d, J = 15.3 Hz, 1H), |
| | | | 6.42 (br d, J = 9.1 Hz, 1H), 6.32 (t, J = 6.7 Hz, 1H), 5.79 (br s, |
| | | | 1H), 4.31-4.21 (m, 1H), 4.08-3.97 (m, 1H), 3.73-3.60 (m, 2H), |
| | | | 2.97 (br s, 2H), 2.91-2.80 (m, 3H), 2.70-2.63 (m, 3H), 2.58 (br |
| | | | d, J = 3.9 Hz, 1H), 2.35-2.25 (m, 2H), 2.11-2.05 (m, 1H), 2.05- |
| | | | 1.96 (m, 2H), 1.90-1.77 (m, 1H), 1.75-1.65 (m, 3H), 1.55- |
| | | | 1.41 (m, 6H), 1.34 (br d, J = 12.9 Hz, 4H), 0.89-0.81 (m, 1H) |
| 585 | 799 | 800 | δ (DMSO-d6) 10.77 (br s, 1H), 8.44 (d, J = 3.5 Hz, 1H), 8.00 (s, |
| | | | 1H), 7.87 (br d, J = 5.1 Hz, 1H), 7.65-7.54 (m, 3H), 7.19 (br d, |
| | | | J = 7.8 Hz, 1H), 6.83 (t, J = 9.4 Hz, 1H), 6.50 (dd, J = 15.0, 2.4 |
| | | | Hz, 1H), 6.41 (dd, J = 8.7, 2.2 Hz, 1H), 5.79 (d, J = 7.6 Hz, 1H), |
| | | | 4.25 (s, 3H), 4.04-3.98 (m, 2H), 3.79 (t, J = 4.9 Hz, 2H), 3.69- |
| | | | 3.59 (m, 1H), 3.55-3.45 (m, 1H), 3.16 (br d, J = 10.4 Hz, 2H), |
| | | | 2.90 (br d, J = 11.4 Hz, 2H), 2.78-2.67 (m, 1H), 2.59 (br d, J = |
| | | | 4.4 Hz, 1H), 2.37-2.23 (m, 1H), 2.19-2.04 (m, 4H), 2.03-1.90 |
| | | | (m, 3H), 1.85-1.72 (m, 5H), 1.67-1.46 (m, 7H), 1.37-1.23 (m, |
| | | | 4H) |
| 586 | 827 | 828 | δ (DMSO-d6) 10.85 (s, 1H), 8.50-8.36 (m, 1H), 8.04-7.96 (m, |
| | | | 1H), 7.91-7.83 (m, 1H), 7.74-7.67 (m, 1H), 7.63-7.57 (m, 2H), |
| | | | 7.19 (br d, J = 7.5 Hz, 1H), 6.89-6.77 (m, 1H), 6.54-6.48 (m, |
| | | | 1H), 6.42 (br d, J = 6.7 Hz, 1H), 5.98-5.76 (m, 1H), 4.29-4.21 |
| | | | (m, 3H), 4.04-3.97 (m, 2H), 3.82-3.76 (m, 2H), 3.69-3.61 (m, |
| | | | 1H), 3.58-3.48 (m, 1H), 3.25-3.11 (m, 2H), 3.07-2.96 (m, 2H), |
| | | | 2.90-2.83 (m, 3H), 2.77-2.66 (m, 1H), 2.59 (br d, J = 3.9 Hz, |
| | | | 1H), 2.46-2.40 (m, 2H), 2.15-2.06 (m, 3H), 2.03-1.96 (m, 2H), |
| | | | 1.92-1.82 (m, 1H), 1.81-1.68 (m, 6H), 1.51-1.40 (m, 3H), 1.37- |
| | | | 1.19 (m, 4H), 0.98-0.77 (m, 4H) |
| 587 | 797 | 798 | δ 10.78 (s, 1H), 8.42 (d, J = 3.7 Hz, 1H), 7.95-7.75 (m, 2H), 7.61 |
| | | | (br d, J = 8.1 Hz, 1H), 7.54 (t, J = 7.8 Hz, 1H), 7.45 (br d, J = 8.6 |
| | | | Hz, 1H), 7.19 (br d, J = 7.5 Hz, 1H), 6.82 (t, J = 9.4 Hz, 1H), 6.49 |
| | | | (dd, J = 2.0, 14.8 Hz, 1H), 6.40 (br d, J = 8.6 Hz, 1H), 5.79 (d, |
| | | | J = 7.6 Hz, 1H), 4.32-4.17 (m, 1H), 3.64 (br t, J = 5.2 Hz, 3H), |
| | | | 3.15 (br d, J = 10.4 Hz, 2H), 2.89 (br d, J = 9.8 Hz, 2H), 2.67 (br |
| | | | d, J = 1.7 Hz, 2H), 2.41 (br t, J = 6.4 Hz, 3H), 2.35-2.26 (m, 3H), |
| | | | 2.17-1.92 (m, 6H), 1.91-1.70 (m, 9H), 1.66-1.46 (m, 6H), 1.38- |
| | | | 1.19 (m, 4H) |
| 588 | 815 | 816 | δ 10.80 (s, 1H), 8.46 (d, J = 3.5 Hz, 1H), 8.01 (s, 1H), 7.94-7.82 |
| | | | (m, 1H), 7.66-7.52 (m, 2H), 7.28 (br d, J = 7.7 Hz, 1H), 6.83 (t, |
| | | | J = 9.4 Hz, 1H), 6.52 (dd, J = 2.4, 15.0 Hz, 1H), 6.43 (dd, J = 2.3, |
| | | | 8.9 Hz, 1H), 5.81 (d, J = 7.6 Hz, 1H), 4.34-4.21 (m, 4H), 4.10- |
| | | | 3.94 (m, 3H), 3.80 (t, J = 5.0 Hz, 2H), 3.67 (br d, J = 9.3 Hz, 2H), |
| | | | 3.06-2.93 (m, 1H), 2.85 (br s, 4H), 2.79-2.72 (m, 1H), 2.70- |
| | | | 2.63 (m, 4H), 2.58 (td, J = 3.7, 17.3 Hz, 2H), 2.32 (s, 2H), 2.10 |
| | | | (td, J = 4.2, 8.7 Hz, 1H), 2.05-1.95 (m, 2H), 1.86 (br dd, J = 4.3, |
| | | | 12.0 Hz, 1H), 1.72 (br d, J = 11.7 Hz, 2H), 1.61-1.26 (m, 9H) |
| 589 | 826 | 827 | δ (DMSO-d6) 10.80 (s, 1H), 8.45 (d, J = 3.6 Hz, 1H), 8.01 (s, |
| | | | 1H), 7.93-7.83 (m, 1H), 7.69 (br t, J = 5.8 Hz, 1H), 7.64-7.57 |
| | | | (m, 2H), 7.21 (br d, J = 7.8 Hz, 1H), 7.03-6.97 (m, 1H), 6.50- |
| | | | 6.41 (m, 2H), 6.05-5.94 (m, 1H), 4.32 (ddd, J = 4.7, 7.1, 11.6 |
| | | | Hz, 1H), 4.25 (s, 2H), 4.02 (dd, J = 4.3, 5.9 Hz, 2H), 3.80 (t, J = |
| | | | 4.9 Hz, 2H), 3.71-3.60 (m, 1H), 2.94-2.86 (m, 4H), 2.81-2.68 |
| | | | (m, 1H), 2.63-2.55 (m, 2H), 2.15-2.06 (m, 4H), 2.01 (br d, J = |
| | | | 10.3 Hz, 2H), 1.95-1.84 (m, 3H), 1.82-1.76 (m, 3H), 1.75 (br |
| | | | s, 3H), 1.63 (br s, 4H), 1.53-1.42 (m, 3H), 1.36-1.23 (m, 3H), |
| | | | 0.95-0.85 (m, 2H), 0.85-0.75 (m, 2H) |
| 590 | 825 | 826 | δ (DMSO-d6) 10.80-10.75 (m, 1H), 8.45-8.39 (m, 1H), 7.89- |
| | | | 7.81 (m, 2H), 7.71-7.65 (m, 1H), 7.56 (t, J = 7.9 Hz, 1H), 7.48- |
| | | | 7.43 (m, 1H), 7.22-7.15 (m, 1H), 6.86-6.78 (m, 1H), 6.55-6.47 |
| | | | (m, 1H), 6.44-6.40 (m, 1H), 5.83-5.76 (m, 1H), 4.29-4.22 (m, |
| | | | 1H), 3.67-3.62 (m, 3H), 2.93-2.81 (m, 6H), 2.78-2.67 (m, 1H), |
| | | | 2.46-2.40 (m, 5H), 2.13-2.06 (m, 3H), 2.00 (br d, J = 9.2 Hz, |
| | | | 2H), 1.93-1.82 (m, 6H), 1.81-1.67 (m, 6H), 1.53-1.39 (m, 4H), |
| | | | 1.35-1.22 (m, 4H), 0.92 (br s, 4H) |
| 591 | 827 | 828 | δ (DMSO-d6) 10.83-10.73 (m, 1H), 8.45 (d, J = 3.6 Hz, 1H), |
| | | | 7.96 (s, 1H), 7.86 (br d, J = 6.6 Hz, 1H), 7.69 (br t, J = 5.6 Hz, |
| | | | 1H), 7.56 (d, J = 1.6 Hz, 1H), 7.20 (br d, J = 7.8 Hz, 1H), 6.84 (t, |
| | | | J = 9.3 Hz, 1H), 6.52 (dd, J = 2.3, 15.1 Hz, 1H), 6.43 (dd, J = 2.0, |
| | | | 8.6 Hz, 1H), 5.81 (d, J = 7.8 Hz, 1H), 4.38 (d, J = 5.4 Hz, 2H), |
| | | | 4.30-4.23 (m, 1H), 3.76-3.60 (m, 3H), 2.93-2.80 (m, 6H), 2.45 |
| | | | (br d, J = 1.5 Hz, 3H), 2.19-2.06 (m, 7H), 2.05-1.97 (m, 2H), |
| | | | 1.91-1.66 (m, 8H), 1.53-1.40 (m, 4H), 1.39-1.22 (m, 4H), 0.95- |
| | | | 0.74 (m, 4H) |
| 592 | 796 | 797 | δ (DMSO-d6) 10.81 (s, 1H), 8.44 (d, J = 3.3 Hz, 1H), 7.90-7.83 |
| | | | (m, 2H), 7.64-7.53 (m, 2H), 7.47 (br d, J = 7.6 Hz, 1H), 7.21 (br |
| | | | d, J = 7.7 Hz, 1H), 7.00 (br t, J = 8.7 Hz, 1H), 6.45 (br d, J = 11.5 |
| | | | Hz, 2H), 6.02 (br d, J = 8.1 Hz, 1H), 4.37-4.28 (m, 1H), 3.66 (br |
| | | | d, J = 5.4 Hz, 3H), 3.55-3.46 (m, 1H), 2.98-2.88 (m, 2H), 2.83- |
| | | | 2.71 (m, 1H), 2.64-2.56 (m, 2H), 2.44 (br t, J = 5.7 Hz, 2H), |
| | | | 2.38-2.28 (m, 3H), 2.15-2.07 (m, 1H), 2.03-1.95 (m, 3H), 1.90 |
| | | | (br d, J = 3.8 Hz, 4H), 1.87 (br d, J = 3.1 Hz, 2H), 1.77 (br d, J = |
| | | | 12.2 Hz, 4H), 1.69-1.59 (m, 4H), 1.42-1.33 (m, 4H), 1.26 (br |
| | | | s, 5H) |
| 593 | 796 | 797 | δ (DMSO-d6) 10.81 (s, 1H), 8.44 (d, J = 3.3 Hz, 1H), 7.90-7.83 |
| | | | (m, 2H), 7.64-7.53 (m, 2H), 7.47 (br d, J = 7.6 Hz, 1H), 7.21 (br |
| | | | d, J = 7.7 Hz, 1H), 7.00 (br t, J = 8.7 Hz, 1H), 6.45 (br d, J = 11.5 |
| | | | Hz, 2H), 6.02 (br d, J = 8.1 Hz, 1H), 4.37-4.28 (m, 1H), 3.66 (br |
| | | | d, J = 5.4 Hz, 3H), 3.55-3.46 (m, 1H), 2.98-2.88 (m, 2H), 2.83- |
| | | | 2.71 (m, 1H), 2.64-2.56 (m, 2H), 2.44 (br t, J = 5.7 Hz, 2H), |
| | | | 2.38-2.28 (m, 3H), 2.15-2.07 (m, 1H), 2.03-1.95 (m, 3H), 1.90 |
| | | | (br d, J = 3.8 Hz, 4H), 1.87 (br d, J = 3.1 Hz, 2H), 1.77 (br d, J = |
| | | | 12.2 Hz, 4H), 1.69-1.59 (m, 4H), 1.42-1.33 (m, 4H), 1.26 (br |
| | | | s, 5H) |
| 594 | 809 | 810 | δ (DMSO-d6) 10.78 (s, 1H), 8.53-8.43 (m, 1H), 8.06 (br d, J = |
| | | | 7.3 Hz, 1H), 7.98 (br s, 1H), 7.78-7.67 (m, 2H), 7.65-7.53 (m, |
| | | | 2H), 7.49-7.42 (m, 1H), 7.24 (br d, J = 7.6 Hz, 1H), 6.84 (br t, |
| | | | J = 9.2 Hz, 1H), 6.56-6.48 (m, 2H), 6.43-6.33 (m, 2H), 5.80 (br |
| | | | d, J = 6.8 Hz, 1H), 5.21-5.06 (m, 1H), 4.30-4.19 (m, 1H), 3.73- |
| | | | 3.62 (m, 1H), 3.53 (br dd, J = 5.7, 3.6 Hz, 1H), 3.18 (br d, J = |
| | | | 9.5 Hz, 2H), 2.81-2.65 (m, 3H), 2.36-2.24 (m, 1H), 2.10 (br |
| | | | dd, J = 8.0, 3.9 Hz, 1H), 2.00-1.85 (m, 5H), 1.85-1.71 (m, 5H), |
| | | | 1.69-1.50 (m, 3H), 1.49-1.31 (m, 7H), 1.28-1.21 (m, 1H), 0.87 |
| | | | (br s, 1H) |
| 595 | 809 | 810 | δ (DMSO-d6) 10.78 (br s, 1H), 8.47 (br d, J = 2.8 Hz, 1H), 8.06 |
| | | | (br d, J = 7.1 Hz, 1H), 7.98 (br s, 1H), 7.77-7.67 (m, 2H), 7.61 |
| | | | (br d, J = 7.6 Hz, 1H), 7.56 (br t, J = 7.5 Hz, 1H), 7.46 (br d, J = |
| | | | 8.1 Hz, 1H), 7.24 (br d, J = 6.8 Hz, 1H), 6.83 (br t, J = 8.8 Hz, |
| | | | 1H), 6.57-6.47 (m, 2H), 6.45-6.34 (m, 2H), 5.88-5.74 (m, 1H), |
| | | | 5.22-5.09 (m, 1H), 4.27 (br d, J = 4.9 Hz, 1H), 3.67 (br d, J = |
| | | | 5.6 Hz, 1H), 3.57-3.47 (m, 1H), 3.20-3.09 (m, 2H), 2.78-2.63 |
| | | | (m, 3H), 2.31 (br dd, J = 10.2, 7.1 Hz, 1H), 2.09 (br d, J = 7.1 Hz, |
| | | | 1H), 2.01-1.84 (m, 5H), 1.76 (br d, J = 10.4 Hz, 4H), 1.68-1.48 |
| | | | (m, 3H), 1.48-1.30 (m, 7H), 1.25 (br s, 2H), 0.95-0.72 (m, 1H) |
| 596 | 785 | 786 | δ 10.77 (br s, 1H), 8.42 (d, J = 3.6 Hz, 1H), 8.38-8.17 (m, 1H), |
| | | | 7.80-7.64 (m, 2H), 7.63-7.49 (m, 2H), 7.16 (d, J = 7.8 Hz, 1H), |
| | | | 6.82 (t, J = 9.3 Hz, 1H), 6.49 (dd, J = 2.3, 14.9 Hz, 1H), 6.40 (dd, |
| | | | J = 2.1, 8.7 Hz, 1H), 5.78 (d, J = 7.6 Hz, 1H), 4.47 (t, J = 8.0 Hz, |
| | | | 2H), 4.24 (ddd, J = 4.8, 7.3, 11.6 Hz, 1H), 4.12 (t, J = 7.9 Hz, |
| | | | 2H), 3.74-3.60 (m, 1H), 3.50 (br dd, J = 3.5, 7.5 Hz, 1H), 3.15 |
| | | | (br d, J = 11.0 Hz, 2H), 2.89 (br d, J = 10.8 Hz, 2H), 2.78-2.66 |
| | | | (m, 1H), 2.63-2.52 (m, 3H), 2.35-2.23 (m, 1H), 2.17-2.06 (m, |
| | | | 3H), 2.04-1.92 (m, 3H), 1.77 (br t, J = 12.0 Hz, 5H), 1.66-1.49 |
| | | | (m, 6H), 1.39-1.20 (m, 4H) |
| 597 | 799 | 800 | δ 10.73 (br dd, J = 1.6, 4.8 Hz, 1H), 8.43 (d, J = 3.5 Hz, 1H), 7.95 |
| | | | (s, 1H), 7.84 (br d, J = 6.5 Hz, 1H), 7.67-7.49 (m, 3H), 7.19 (br |
| | | | d, J = 7.9 Hz, 1H), 6.82 (t, J = 9.4 Hz, 1H), 6.50 (dd, J = 2.1, 14.9 |
| | | | Hz, 1H), 6.41 (dd, J = 1.7, 8.8 Hz, 1H), 5.78 (br d, J = 7.6 Hz, |
| | | | 1H), 4.37 (t, J = 5.3 Hz, 2H), 4.30-4.16 (m, 1H), 3.71 (br t, J = |
| | | | 6.0 Hz, 2H), 3.68-3.58 (m, 1H), 3.55-3.41 (m, 2H), 3.16 (br d, |
| | | | J = 10.8 Hz, 2H), 2.90 (br d, J = 11.0 Hz, 2H), 2.80-2.67 (m, |
| | | | 1H), 2.63-2.53 (m, 3H), 2.36-2.24 (m, 1H), 2.18-2.05 (m, 5H), |
| | | | 2.03-1.93 (m, 2H), 1.89-1.71 (m, 5H), 1.69-1.45 (m, 6H), 1.43- |
| | | | 1.18 (m, 4H). |
| 598 | 829 | 830 | δ 10.79 (s, 1H), 9.95-9.77 (m, 1H), 8.47 (d, J = 3.6 Hz, 1H), 8.05 |
| | | | (br d, J = 7.6 Hz, 1H), 7.97 (br s, 1H), 7.88-7.80 (m, 1H), 7.75- |
| | | | 7.66 (m, 2H), 7.63-7.51 (m, 2H), 7.28-7.19 (m, 1H), 6.89 (t, |
| | | | J = 9.3 Hz, 1H), 6.58-6.49 (m, 2H), 6.44 (br d, J = 8.6 Hz, 1H), |
| | | | 6.40-6.32 (m, 1H), 4.29 (br dd, J = 4.8, 11.6 Hz, 1H), 3.72-3.60 |
| | | | (m, 1H), 3.41-3.40 (m, 3H), 3.31-3.06 (m, 4H), 2.96-2.88 (m, |
| | | | 1H), 2.87-2.65 (m, 3H), 2.33 (br d, J = 1.6 Hz, 1H), 2.10-1.76 |
| | | | (m, 12H), 1.40-1.15 (m, 5H) |
| 599 | 829 | 830 | δ 11.97-11.74 (m, 1H), 10.79 (s, 1H), 8.42 (d, J = 3.5 Hz, 1H), |
| | | | 8.32 (s, 1H), 7.94-7.65 (m, 4H), 7.55 (t, J = 7.8 Hz, 1H), 7.44 |
| | | | (dd, J = 1.9, 6.4 Hz, 1H), 7.16 (br d, J = 7.6 Hz, 1H), 6.88 (br t, |
| | | | J = 9.3 Hz, 1H), 6.59-6.40 (m, 2H), 6.33 (t, J = 6.7 Hz, 1H), 6.08- |
| | | | 5.71 (m, 1H), 4.29 (br dd, J = 4.5, 11.4 Hz, 1H), 3.71-3.58 (m, |
| | | | 2H), 3.48 (br s, 6H), 3.17-3.07 (m, 1H), 2.88-2.65 (m, 3H), |
| | | | 2.64-2.56 (m, 1H), 2.43-2.30 (m, 1H), 2.13-1.94 (m, 5H), 1.92- |
| | | | 1.74 (m, 5H), 1.43-1.16 (m, 6H) |
| 600 | 798 | 799 | δ (DMSO-d6) 10.80 (s, 1H), 8.44 (d, J = 3.6 Hz, 1H), 7.96 (s, |
| | | | 1H), 7.88-7.83 (m, 1H), 7.60-7.52 (m, 2H), 7.26 (br d, J = 7.7 |
| | | | Hz, 1H), 6.99 (br t, J = 8.4 Hz, 1H), 6.49-6.42 (m, 2H), 6.12- |
| | | | 5.94 (m, 1H), 4.43-4.36 (m, 3H), 4.31 (ddd, J = 4.8, 7.2, 11.8 |
| | | | Hz, 1H), 4.00-3.91 (m, 1H), 3.72 (t, J = 6.1 Hz, 2H), 3.68-3.60 |
| | | | (m, 1H), 3.08-2.89 (m, 3H), 2.82-2.65 (m, 2H), 2.65-2.55 (m, |
| | | | 3H), 2.19-2.09 (m, 4H), 2.09-2.05 (m, 1H), 2.01 (br d, J = 9.8 |
| | | | Hz, 3H), 1.93-1.84 (m, 2H), 1.84-1.75 (m, 3H), 1.75-1.66 (m, |
| | | | 5H), 1.46 (dt, J = 2.3, 4.1 Hz, 2H), 1.42-1.32 (m, 2H), 1.27- |
| | | | 1.11 (m, 1H), 1.09-0.86 (m, 2H) |
| 601 | 798 | 799 | δ (DMSO-d6) 10.79 (s, 1H), 8.44 (d, J = 3.6 Hz, 1H), 7.96 (s, |
| | | | 1H), 7.88-7.82 (m, 1H), 7.60-7.52 (m, 2H), 7.29-7.23 (m, 1H), |
| | | | 7.03-6.95 (m, 1H), 6.49-6.41 (m, 2H), 6.00 (d, J = 7.9 Hz, 1H), |
| | | | 4.42-4.35 (m, 3H), 4.34-4.27 (m, 1H), 3.97-3.91 (m, 1H), 3.72 |
| | | | (t, J = 6.1 Hz, 2H), 3.68-3.61 (m, 1H), 3.05-2.96 (m, 1H), 2.95- |
| | | | 2.89 (m, 2H), 2.84-2.64 (m, 2H), 2.60-2.56 (m, 2H), 2.16- |
| | | | 2.11 (m, 4H), 2.09-2.05 (m, 1H), 2.02-1.97 (m, 2H), 1.93 (br |
| | | | dd, J = 4.1, 10.6 Hz, 2H), 1.87 (br dd, J = 4.4, 12.1 Hz, 1H), 1.81- |
| | | | 1.76 (m, 2H), 1.71 (br d, J = 12.3 Hz, 3H), 1.63 (br s, 4H), 1.52- |
| | | | 1.42 (m, 2H), 1.40-1.32 (m, 2H), 1.20-1.12 (m, 1H), 1.03- |
| | | | 0.86 (m, 2H) |
| 602 | 793 | 794 | δ (DMSO-d6) 10.77 (s, 1H), 8.46 (d, J = 3.6 Hz, 1H), 8.05 (br d, |
| | | | J = 7.3 Hz, 1H), 7.97 (s, 1H), 7.75-7.67 (m, 2H), 7.62-7.53 (m, |
| | | | 3H), 7.22 (br d, J = 7.5 Hz, 1H), 6.81 (t, J = 9.4 Hz, 1H), 6.54- |
| | | | 6.48 (m, 2H), 6.44-6.32 (m, 2H), 5.79 (d, J = 7.5 Hz, 1H), 4.29- |
| | | | 4.21 (m, 1H), 3.70-3.60 (m, 1H), 3.52-3.43 (m, 1H), 2.83 (br |
| | | | s, 3H), 2.76-2.68 (m, 1H), 2.61 (br s, 4H), 2.29-2.21 (m, 1H), |
| | | | 2.09 (td, J = 4.0, 8.5 Hz, 1H), 2.02-1.93 (m, 3H), 1.86 (br d, J = |
| | | | 5.1 Hz, 3H), 1.77 (br t, J = 11.9 Hz, 4H), 1.41-1.18 (m, 10H) |
| 603 | 793 | 794 | δ (DMSO-d6) 10.77 (s, 1H), 8.46 (d, J = 3.6 Hz, 1H), 8.05 (br d, |
| | | | J = 7.3 Hz, 1H), 7.97 (s, 1H), 7.75-7.67 (m, 2H), 7.62-7.53 (m, |
| | | | 3H), 7.22 (br d, J = 7.5 Hz, 1H), 6.81 (t, J = 9.4 Hz, 1H), 6.54- |
| | | | 6.48 (m, 2H), 6.44-6.32 (m, 2H), 5.79 (d, J = 7.5 Hz, 1H), 4.29- |
| | | | 4.21 (m, 1H), 3.70-3.60 (m, 1H), 3.52-3.43 (m, 1H), 2.83 (br |
| | | | s, 3H), 2.76-2.68 (m, 1H), 2.61 (br s, 4H), 2.29-2.21 (m, 1H), |
| | | | 2.09 (td, J = 4.0, 8.5 Hz, 1H), 2.02-1.93 (m, 3H), 1.86 (br d, J = |
| | | | 5.1 Hz, 3H), 1.77 (br t, J = 11.9 Hz, 4H), 1.41-1.18 (m, 10H) |
| 604 | 776 | 777 | δ (DMSO-d6) 10.78 (s, 1H), 8.45 (d, J = 3.8 Hz, 1H), 8.26 (br s, |
| | | | 1H), 8.15 (s, 1H), 7.96 (br d, J = 7.5 Hz, 1H), 7.85 (d, J = 7.8 Hz, |
| | | | 1H), 7.74-7.70 (m, 2H), 7.67-7.59 (m, 2H), 7.52 (t, J = 7.6 Hz, |
| | | | 2H), 7.46-7.39 (m, 1H), 7.21 (d, J = 7.8 Hz, 1H), 6.82 (t, J = 9.4 |
| | | | Hz, 1H), 6.51 (dd, J = 15.1, 2.4 Hz, 1H), 6.46-6.39 (m, 1H), 5.82 |
| | | | (d, J = 7.8 Hz, 1H), 4.26 (ddd, J = 11.6, 7.2, 4.9 Hz, 1H), 3.71- |
| | | | 3.60 (m, 1H), 3.50 (br dd, J = 7.4, 4.2 Hz, 1H), 2.86 (br s, 3H), |
| | | | 2.78-2.73 (m, 1H), 2.69 (br d, J = 2.9 Hz, 3H), 2.60 (br t, J = 4.1 |
| | | | Hz, 1H), 2.57-2.53 (m, 1H), 2.39-2.29 (m, 1H), 2.09 (td, J = |
| | | | 8.6, 4.3 Hz, 1H), 2.04-1.96 (m, 3H), 1.93-1.85 (m, 3H), 1.85- |
| | | | 1.73 (m, 5H), 1.44-1.32 (m, 4H), 1.30-1.19 (m, 4H) |
| 605 | 785 | 786 | δ (DMSO-d6) 10.78 (s, 1H), 8.39 (d, J = 3.8 Hz, 1H), 8.20 (s, |
| | | | 1H), 7.64-7.50 (m, 2H), 7.43-7.35 (m, 2H), 7.17-7.10 (m, 2H), |
| | | | 6.81 (t, J = 9.4 Hz, 1H), 6.51 (dd, J = 14.9, 2.3 Hz, 1H), 6.42 (dd, |
| | | | J = 8.7, 2.2 Hz, 1H), 5.80 (d, J = 7.6 Hz, 1H), 4.26 (ddd, J = 11.5, |
| | | | 7.2, 4.8 Hz, 1H), 3.80-3.76 (m, 4H), 3.65-3.58 (m, 1H), 3.49 |
| | | | (br dd, J = 7.7, 3.7 Hz, 1H), 3.18-3.14 (m, 4H), 3.04-2.90 (m, |
| | | | 1H), 2.84 (br s, 3H), 2.78-2.70 (m, 1H), 2.63-2.58 (m, 4H), |
| | | | 2.57-2.53 (m, 1H), 2.31-2.22 (m, 1H), 2.09 (ddd, J = 13.1, 8.6, |
| | | | 4.6 Hz, 1H), 2.04-1.95 (m, 3H), 1.90-1.83 (m, 3H), 1.78 (br t, |
| | | | J = 13.4 Hz, 4H), 1.44-1.31 (m, 4H), 1.29-1.18 (m, 4H) |
| 632 | 809 | 810 | δ (DMSO-d6) 10.80 (br s, 1H), 8.45 (br d, J = 1.3 Hz, 1H), 8.26 |
| | | | (s, 1H), 7.86 (d, J = 7.9 Hz, 1H), 7.78-7.65 (m, 3H), 7.62-7.45 |
| | | | (m, 4H), 6.82 (t, J = 9.4 Hz, 1H), 6.55-6.48 (m, 2H), 6.46-6.40 |
| | | | (m, 1H), 6.36 (t, J = 6.5 Hz, 1H), 5.82 (br d, J = 7.6 Hz, 1H), 4.29- |
| | | | 4.22 (m, 1H), 3.70-3.62 (m, 2H), 3.48 (br dd, J = 7.4, 3.6 Hz, |
| | | | 2H), 2.84 (br s, 3H), 2.78-2.70 (m, 1H), 2.62 (br s, 3H), 2.57- |
| | | | 2.54 (m, 1H), 2.27 (br t, J = 11.2 Hz, 1H), 2.14-2.06 (m, 1H), |
| | | | 2.00-1.91 (m, 3H), 1.90-1.83 (m, 3H), 1.77 (br d, J = 0.9 Hz, |
| | | | 4H), 1.42-1.31 (m, 4H), 1.29-1.19 (m, 4H) |
| 633 | 806 | 807 | δ (DMSO-d6) 10.87-10.72 (m, 1H), 8.48 (d, J = 3.6 Hz, 1H), |
| | | | 8.05 (br d, J = 7.3 Hz, 1H), 7.98 (s, 1H), 7.74-7.67 (m, 2H), 7.63- |
| | | | 7.58 (m, 1H), 7.55 (ddd, J = 2.0, 6.8, 9.1 Hz, 1H), 7.35 (br d, |
| | | | J = 7.6 Hz, 1H), 7.01-6.95 (m, 1H), 6.52 (d, J = 9.1 Hz, 1H), 6.47- |
| | | | 6.40 (m, 2H), 6.39-6.34 (m, 1H), 6.00 (br d, J = 7.9 Hz, 1H), |
| | | | 4.76 (quin, J = 7.2 Hz, 1H), 4.34-4.26 (m, 1H), 3.96-3.85 (m, |
| | | | 3H), 2.97-2.66 (m, 5H), 2.62-2.54 (m, 3H), 2.47-2.39 (m, 1H), |
| | | | 2.30-2.21 (m, 1H), 2.18-2.10 (m, 1H), 2.09-2.01 (m, 2H), 2.01- |
| | | | 1.94 (m, 1H), 1.92-1.77 (m, 5H), 1.77-1.67 (m, 2H), 1.66- |
| | | | 1.54 (m, 4H), 1.43-1.30 (m, 2H), 1.27-1.14 (m, 1H) |
| 634 | 814 | 815 | δ 10.49 (s, 1H), 8.45 (d, J = 3.6 Hz, 1H), 8.25 (s, 1H), 7.96 (br d, |
| | | | J = 7.1 Hz, 1H), 7.84 (d, J = 7.9 Hz, 1H), 7.71 (d, J = 7.8 Hz, 2H), |
| | | | 7.64 (t, J = 7.8 Hz, 1H), 7.51 (t, J = 7.6 Hz, 2H), 7.47-7.38 (m, |
| | | | 2H), 7.22 (d, J = 7.6 Hz, 1H), 6.91 (br d, J = 9.1 Hz, 1H), 6.82 (s, |
| | | | 1H), 4.50 (br s, 1H), 3.98 (br d, J = 12.1 Hz, 2H), 3.93-3.85 (m, |
| | | | 5H), 3.82-3.70 (m, 1H), 3.22 (br s, 6H), 2.86-2.69 (m, 4H), |
| | | | 2.20 (br d, J = 6.6 Hz, 2H), 2.10-1.88 (m, 5H), 1.73 (br d, J = |
| | | | 12.1 Hz, 3H), 1.56-1.27 (m, 5H), 1.12-0.88 (m, 2H) |
| 635 | 829 | 830 | δ 10.53 (s, 1H), 8.45 (d, J = 3.6 Hz, 1H), 8.24 (s, 1H), 7.95 (br d, |
| | | | J = 7.4 Hz, 1H), 7.83 (d, J = 7.9 Hz, 1H), 7.70 (d, J = 7.5 Hz, 2H), |
| | | | 7.63 (t, J = 7.8 Hz, 1H), 7.56-7.47 (m, 3H), 7.45-7.38 (m, 2H), |
| | | | 7.22 (d, J = 7.6 Hz, 1H), 7.02 (d, J = 8.5 Hz, 1H), 4.51 (br s, 1H), |
| | | | 4.20 (s, 1H), 3.95 (s, 3H), 3.89 (t, J = 6.6 Hz, 2H), 3.82-3.62 (m, |
| | | | 3H), 3.24-3.07 (m, 2H), 3.03 (br d, J = 11.0 Hz, 2H), 2.74 (t, |
| | | | J = 6.6 Hz, 2H), 2.62-2.55 (m, 1H), 2.35-2.24 (m, 4H), 2.05- |
| | | | 1.92 (m, 4H), 1.84-1.67 (m, 4H), 1.56-1.47 (m, 3H), 1.46 (br |
| | | | s, 5H) |
| 636 | 813 | 814 | δ 10.53 (s, 1H), 8.45 (d, J = 3.5 Hz, 1H), 8.25 (br s, 1H), 7.96 (br |
| | | | d, J = 6.8 Hz, 1H), 7.84 (br d, J = 7.9 Hz, 1H), 7.71 (br d, J = 7.5 |
| | | | Hz, 2H), 7.67-7.61 (m, 1H), 7.57-7.48 (m, 3H), 7.47-7.39 (m, |
| | | | 2H), 7.22 (br d, J = 7.6 Hz, 1H), 7.03 (br d, J = 8.6 Hz, 1H), 4.50 |
| | | | (br s, 1H), 4.03-3.94 (m, 5H), 3.90 (br t, J = 6.6 Hz, 2H), 3.74 |
| | | | (br d, J = 4.0 Hz, 1H), 2.96 (br d, J = 10.3 Hz, 2H), 2.75 (br t, J = |
| | | | 6.6 Hz, 3H), 2.69-2.60 (m, 1H), 2.17 (br d, J = 6.1 Hz, 2H), 1.99 |
| | | | (br d, J = 7.6 Hz, 6H), 1.84-1.69 (m, 7H), 1.43 (br s, 4H), 1.23 |
| | | | (s, 1H), 0.98 (q, J = 10.8 Hz, 2H) |
| 637 | 807 | 808 | δ 10.90-10.62 (m, 1H), 8.47 (d, J = 3.6 Hz, 1H), 8.04 (d, J = 8.1 |
| | | | Hz, 1H), 7.97 (s, 1H), 7.76-7.67 (m, 2H), 7.63-7.49 (m, 2H), |
| | | | 7.33 (d, J = 7.6 Hz, 1H), 6.81 (t, J = 9.3 Hz, 1H), 6.57-6.46 (m, |
| | | | 2H), 6.43-6.32 (m, 2H), 5.77 (d, J = 7.5 Hz, 1H), 4.74 (t, J = 7.1 |
| | | | Hz, 1H), 4.23 (ddd, J = 5.0, 7.0, 11.5 Hz, 1H), 3.90 (br d, J = 13.0 |
| | | | Hz, 3H), 3.11 (s, 2H), 3.08-3.02 (m, 4H), 3.00-2.86 (m, 2H), |
| | | | 2.80-2.64 (m, 2H), 2.60-2.55 (m, 1H), 2.48-2.39 (m, 3H), 2.13- |
| | | | 2.03 (m, 3H), 2.02-1.94 (m, 1H), 1.90-1.80 (m, 3H), 1.65 (br |
| | | | d, J = 9.4 Hz, 2H), 1.45-1.30 (m, 2H), 1.23 (q, J = 9.6 Hz, 2H) |
| 638 | 829 | 830 | δ (DMSO-d6) 10.88 (s, 1H), 8.48 (d, J = 3.5 Hz, 1H), 8.07 (br d, |
| | | | J = 7.5 Hz, 1H), 7.99 (s, 1H), 7.77-7.66 (m, 2H), 7.61 (d, J = 8.3 |
| | | | Hz, 2H), 7.57-7.52 (m, 1H), 7.45 (s, 1H), 7.26 (d, J = 7.6 Hz, |
| | | | 1H), 7.04 (d, J = 8.4 Hz, 1H), 6.52 (d, J = 9.1 Hz, 1H), 6.36 (dt, |
| | | | J = 6.7, 1.1 Hz, 1H), 4.50 (br s, 1H), 4.33 (dd, J = 9.7, 5.1 Hz, 1H), |
| | | | 3.98 (s, 3H), 3.75 (br dd, J = 4.8, 2.7 Hz, 1H), 2.97 (br d, J = 10.4 |
| | | | Hz, 2H), 2.83-2.70 (m, 2H), 2.69-2.59 (m, 3H), 2.35 (tdd, J = |
| | | | 13.8, 9.2, 4.7 Hz, 1H), 2.20-2.13 (m, 3H), 2.07-1.91 (m, 7H), |
| | | | 1.83-1.67 (m, 8H), 1.46-1.36 (m, 4H), 1.05-0.94 (m, 2H) |
| 639 | 829 | 830 | δ (DMSO-d6) 10.87 (s, 1H), 8.47 (d, J = 3.6 Hz, 1H), 8.07 (br d, |
| | | | J = 7.8 Hz, 1H), 7.98 (s, 1H), 7.74-7.68 (m, 2H), 7.60 (d, J = 8.3 |
| | | | Hz, 2H), 7.57-7.51 (m, 1H), 7.45 (s, 1H), 7.25 (d, J = 7.8 Hz, |
| | | | 1H), 7.04 (d, J = 8.5 Hz, 1H), 6.52 (d, J = 9.1 Hz, 1H), 6.36 (dt, |
| | | | J = 6.7, 1.1, Hz, 1H), 4.50 (br d, J = 4.5 Hz, 1H), 4.33 (dd, J = 9.7, |
| | | | 5.1 Hz, 1H), 3.97 (s, 3H), 3.80-3.68 (m, 1H), 2.97 (br d, J = 10.6 |
| | | | Hz, 2H), 2.82-2.71 (m, 2H), 2.69-2.57 (m, 4H), 2.55 (s, 1H), |
| | | | 2.41-2.31 (m, 2H), 2.20-2.13 (m, 3H), 2.04-1.94 (m, 6H), 1.78 |
| | | | (br s, 3H), 1.73 (br d, J = 11.6 Hz, 3H), 1.46-1.37 (m, 4H), 1.04- |
| | | | 0.91 (m, 2H) |
| 640 | 805 | 806 | δ (DMSO-d6) 10.79 (br s, 1H), 8.47 (d, J = 3.6 Hz, 1H), 8.36 (br |
| | | | s, 1H), 8.24 (s, 1H), 7.96 (br d, J = 7.6 Hz, 1H), 7.84 (br d, J = |
| | | | 7.6 Hz, 1H), 7.71 (br d, J = 7.3 Hz, 2H), 7.67-7.62 (m, 1H), 7.51 |
| | | | (t, J = 7.6 Hz, 2H), 7.45-7.38 (m, 1H), 7.32 (br d, J = 7.6 Hz, |
| | | | 1H), 6.99 (br t, J = 8.6 Hz, 1H), 6.47-6.43 (m, 1H), 5.99 (br d, |
| | | | J = 7.5 Hz, 1H), 4.34-4.26 (m, 2H), 3.96 (br d, J = 13.4 Hz, 3H), |
| | | | 2.92-2.86 (m, 3H), 2.77-2.67 (m, 3H), 2.60-2.56 (m, 2H), 2.09 |
| | | | (br d, J = 6.8 Hz, 3H), 1.96-1.87 (m, 5H), 1.85-1.78 (m, 3H), |
| | | | 1.72 (br d, J = 10.8 Hz, 2H), 1.62 (br s, 4H), 1.46-1.37 (m, 3H), |
| | | | 1.03-0.93 (m, 2H), 0.90-0.83 (m, 2H) |
| 641 | 838 | 839 | δ (DMSO-d6) 8.50-8.46 (m, 1H), 8.07 (br d, J = 7.8 Hz, 1H), |
| | | | 7.99 (s, 1H), 7.75-7.68 (m, 2H), 7.61 (br d, J = 7.8 Hz, 1H), 7.56 |
| | | | (ddd, J = 2.1, 6.7, 9.1 Hz, 1H), 7.27 (br d, J = 8.0 Hz, 1H), 7.02- |
| | | | 6.96 (m, 1H), 6.53 (d, J = 9.3 Hz, 1H), 6.48-6.41 (m, 2H), 6.39- |
| | | | 6.35 (m, 1H), 6.01 (d, J = 7.6 Hz, 1H), 5.39-5.10 (m, 1H), 4.55- |
| | | | 4.44 (m, 1H), 4.35-4.25 (m, 1H), 3.72-3.65 (m, 4H), 3.02 (br |
| | | | d, J = 10.1 Hz, 2H), 2.79-2.68 (m, 3H), 2.13-2.06 (m, 1H), 2.01- |
| | | | 1.94 (m, 6H), 1.67 (br dd, J = 1.8, 12.9 Hz, 3H), 1.56 (br s, 4H), |
| | | | 1.50 (br d, J = 12.5 Hz, 3H), 1.46-1.40 (m, 4H), 1.39-1.33 (m, |
| | | | 3H) |
| 642 | 829 | 830 | δ 10.85 (s, 1H), 8.46 (d, J = 3.5 Hz, 1H), 8.26 (s, 1H), 7.97 (br d, |
| | | | J = 6.6 Hz, 1H), 7.84 (d, J = 7.6 Hz, 1H), 7.72 (d, J = 7.5 Hz, 2H), |
| | | | 7.65 (t, J = 7.8 Hz, 1H), 7.58-7.45 (m, 3H), 7.45-7.37 (m, 1H), |
| | | | 7.23 (d, J = 7.8 Hz, 1H), 6.91 (br d, J = 8.6 Hz, 1H), 6.83 (s, 1H), |
| | | | 4.59-4.43 (m, 1H), 4.26 (br dd, J = 5.1, 9.3 Hz, 2H), 3.89 (s, |
| | | | 3H), 3.68 (br d, J = 13.1 Hz, 3H), 3.21 (br s, 6H), 2.71 (br s, 4H), |
| | | | 2.66-2.57 (m, 2H), 2.43-2.24 (m, 3H), 2.21-2.13 (m, 1H), 2.06- |
| | | | 1.90 (m, 4H), 1.58-1.34 (m, 8H) |
| 643 | 813 | 814 | δ 10.86 (s, 1H), 8.46 (d, J = 3.5 Hz, 1H), 8.26 (s, 1H), 8.20 (s, |
| | | | 1H), 7.97 (br d, J = 7.5 Hz, 1H), 7.85 (br d, J = 7.8 Hz, 1H), 7.72 |
| | | | (d, J = 7.4 Hz, 2H), 7.65 (t, J = 7.6 Hz, 1H), 7.57-7.46 (m, 3H), |
| | | | 7.46-7.37 (m, 1H), 7.24 (br d, J = 7.8 Hz, 1H), 6.93 (br d, J = |
| | | | 8.9 Hz, 1H), 6.85 (s, 1H), 4.51 (br d, J = 2.6 Hz, 1H), 4.26 (dd, |
| | | | J = 4.9, 9.1 Hz, 1H), 3.98 (br d, J = 11.4 Hz, 2H), 3.90 (s, 3H), 3.84- |
| | | | 3.66 (m, 2H), 3.22 (br s, 5H), 2.87-2.68 (m, 3H), 2.65-2.58 |
| | | | (m, 2H), 2.35-2.27 (m, 1H), 2.24-2.10 (m, 3H), 1.98 (br d, J = |
| | | | 9.5 Hz, 4H), 1.74 (br d, J = 12.5 Hz, 3H), 1.41 (br d, J = 14.4 Hz, |
| | | | 5H), 1.00 (br dd, J = 1.4, 12.1 Hz, 2H) |
| 644 | 822 | 823 | δ (DMSO-d6) 8.48 (br s, 1H), 8.10-8.02 (m, 1H), 8.00-7.95 (m, |
| | | | 1H), 7.75-7.67 (m, 2H), 7.63-7.58 (m, 1H), 7.57-7.50 (m, 1H), |
| | | | 7.35 (br dd, J = 7.9, 2.6 Hz, 1H), 7.29-7.19 (m, 1H), 7.04-6.88 |
| | | | (m, 1H), 6.52 (br d, J = 9.0 Hz, 1H), 6.48-6.40 (m, 1H), 6.38- |
| | | | 6.31 (m, 1H), 3.93 (br dd, J = 10.9, 1.5 Hz, 3H), 3.83 (br d, J = |
| | | | 5.4 Hz, 2H), 2.94-2.86 (m, 4H), 2.09 (br s, 3H), 1.93-1.87 (m, |
| | | | 4H), 1.83-1.77 (m, 3H), 1.75-1.69 (m, 3H), 1.67-1.54 (m, 6H), |
| | | | 1.46-1.33 (m, 4H), 1.24 (br s, 1H), 1.01-0.95 (m, 2H), 0.89- |
| | | | 0.83 (m, 2H) |
| 645 | 785 | 786 | δ (DMSO-d6) 8.40 (d, J = 4.2 Hz, 1H), 7.67-7.51 (m, 2H), 7.41 |
| | | | (br d, J = 5.9 Hz, 2H), 7.15 (br d, J = 7.7 Hz, 2H), 6.84 (t, J = 9.4 |
| | | | Hz, 1H), 6.51 (dd, J = 2.5, 14.9 Hz, 1H), 6.42 (br d, J = 8.6 Hz, |
| | | | 1H), 5.80 (d, J = 7.7 Hz, 1H), 4.30-4.22 (m, 1H), 3.82-3.76 (m, |
| | | | 4H), 3.65 (br s, 1H), 3.51 (br s, 1H), 3.20-3.15 (m, 5H), 2.91 (br |
| | | | d, J = 10.5 Hz, 2H), 2.86-2.68 (m, 2H), 2.61-2.55 (m, 3H), 2.36- |
| | | | 2.27 (m, 1H), 2.18-1.96 (m, 6H), 1.88-1.74 (m, 5H), 1.67- |
| | | | 1.50 (m, 6H), 1.41-1.23 (m, 4H) |
| 646 | 828 | 829 | δ 10.86 (s, 1H), 8.44 (d, J = 3.7 Hz, 1H), 8.24 (s, 1H), 7.95 (br d, |
| | | | J = 7.6 Hz, 1H), 7.83 (d, J = 8.0 Hz, 1H), 7.73-7.68 (m, 2H), |
| | | | 7.66-7.55 (m, 2H), 7.50 (t, J = 7.6 Hz, 2H), 7.44-7.37 (m, 2H), |
| | | | 7.22 (d, J = 7.6 Hz, 1H), 7.02 (d, J = 8.6 Hz, 1H), 4.50 (br d, J = |
| | | | 3.5 Hz, 1H), 4.31 (dd, J = 5.1, 9.7 Hz, 1H), 4.20 (s, 1H), 3.95 (s, |
| | | | 3H), 3.82-3.64 (m, 3H), 3.22-2.97 (m, 4H), 2.73-2.54 (m, 3H), |
| | | | 2.33-2.25 (m, 4H), 2.20-2.10 (m, 1H), 1.98 (br d, J = 6.7 Hz, |
| | | | 4H), 1.84-1.70 (m, 4H), 1.55-1.21 (m, 9H) |
| 647 | 812 | 813 | δ 10.87 (s, 1H), 8.45 (d, J = 3.6 Hz, 1H), 8.25 (s, 1H), 8.18 (d, |
| | | | J = 1.3 Hz, 1H), 7.96 (br d, J = 6.9 Hz, 1H), 7.84 (d, J = 7.6 Hz, |
| | | | 1H), 7.71 (d, J = 7.5 Hz, 2H), 7.67-7.57 (m, 2H), 7.55-7.46 (m, |
| | | | 2H), 7.45-7.37 (m, 2H), 7.22 (d, J = 7.6 Hz, 1H), 7.03 (d, J = 8.4 |
| | | | Hz, 1H), 4.50 (br s, 1H), 4.32 (dd, J = 5.1, 9.7 Hz, 1H), 4.05- |
| | | | 3.90 (m, 5H), 3.74 (br s, 1H), 2.97 (br d, J = 10.9 Hz, 2H), 2.82- |
| | | | 2.54 (m, 5H), 2.34 (dtd, J = 4.9, 9.4, 13.9 Hz, 1H), 2.23-2.12 (m, |
| | | | 3H), 2.08-1.90 (m, 6H), 1.82-1.68 (m, 6H), 1.42 (br s, 4H), |
| | | | 1.14-0.86 (m, 3H) |
| 648 | 830 | 831 | δ 11.29-10.46 (m, 1H), 8.47 (d, J = 3.6 Hz, 1H), 8.06 (br d, J = |
| | | | 7.5 Hz, 1H), 7.98 (s, 1H), 7.76-7.65 (m, 2H), 7.64-7.50 (m, |
| | | | 2H), 7.32-7.19 (m, 1H), 6.99 (br t, J = 8.8 Hz, 1H), 6.52 (d, J = |
| | | | 9.1 Hz, 1H), 6.47-6.39 (m, 2H), 6.35 (dt, J = 1.1, 6.7 Hz, 1H), |
| | | | 5.98 (br d, J = 7.8 Hz, 1H), 4.51 (br s, 1H), 4.36-4.24 (m, 1H), |
| | | | 4.17-3.99 (m, 2H), 3.81-3.70 (m, 1H), 3.13-2.92 (m, 4H), 2.73 |
| | | | (ddd, J = 5.1, 12.1, 17.5 Hz, 1H), 2.64-2.54 (m, 4H), 2.14-2.04 |
| | | | (m, 1H), 1.96 (br s, 4H), 1.87-1.72 (m, 3H), 1.68-1.54 (m, 4H), |
| | | | 1.52-1.14 (m, 5H) |
| 649 | 776 | 777 | δ (DMSO-d6) 8.44 (d, J = 3.5 Hz, 1H), 8.25 (br s, 1H), 7.95 (br |
| | | | d, J = 7.4 Hz, 1H), 7.84 (br d, J = 7.0 Hz, 1H), 7.71 (d, J = 7.5 |
| | | | Hz, 2H), 7.68-7.57 (m, 2H), 7.52 (t, J = 7.6 Hz, 2H), 7.46-7.39 |
| | | | (m, 1H), 7.19 (br d, J = 7.5 Hz, 1H), 6.82 (t, J = 9.3 Hz, 1H), 6.49 |
| | | | (br d, J = 14.4 Hz, 1H), 6.44-6.37 (m, 1H), 5.78 (br d, J = 7.5 |
| | | | Hz, 1H), 4.29-4.21 (m, 1H), 3.16 (br d, J = 10.8 Hz, 3H), 2.90 |
| | | | (br d, J = 10.4 Hz, 3H), 2.72 (br d, J = 12.0 Hz, 2H), 2.35-2.25 |
| | | | (m, 2H), 2.18-2.05 (m, 4H), 2.01 (br dd, J = 4.6, 7.6 Hz, 3H), |
| | | | 1.85-1.70 (m, 5H), 1.67-1.51 (m, 6H), 1.38-1.23 (m, 4H) |
| 650 | 793 | 794 | δ (DMSO-d6) 8.52 (d, J = 3.7 Hz, 1H), 8.10 (br d, J = 7.0 Hz, |
| | | | 1H), 8.02 (s, 1H), 7.78-7.71 (m, 2H), 7.64 (br d, J = 8.6 Hz, 1H), |
| | | | 7.59 (ddd, J = 9.1, 6.7, 2.1 Hz, 1H), 7.40 (br d, J = 7.7 Hz, 1H), |
| | | | 6.85 (t, J = 9.3 Hz, 1H), 6.59-6.51 (m, 2H), 6.46 (br d, J = 8.9 |
| | | | Hz, 1H), 6.43-6.38 (m, 1H), 5.83 (d, J = 8.0 Hz, 1H), 4.38-4.25 |
| | | | (m, 2H), 4.03-3.88 (m, 2H), 3.34 (br d, J = 3.8 Hz, 3H), 2.90- |
| | | | 2.85 (m, 3H), 2.81-2.76 (m, 1H), 2.73-2.70 (m, 1H), 2.64 (br |
| | | | d, J = 4.9 Hz, 5H), 2.39-2.36 (m, 1H), 2.24 (br d, J = 6.7 Hz, |
| | | | 2H), 2.17-2.09 (m, 1H), 2.02-1.91 (m, 2H), 1.89-1.78 (m, 4H), |
| | | | 1.70-1.59 (m, 1H), 1.51-1.20 (m, 4H), 1.08-0.96 (m, 2H) |
| 651 | 777 | 778 | δ 10.79 (s, 1H), 8.46 (d, J = 3.6 Hz, 1H), 8.25 (s, 1H), 7.97 (br d, |
| | | | J = 7.4 Hz, 1H), 7.84 (br d, J = 8.0 Hz, 1H), 7.71 (d, J = 7.3 Hz, |
| | | | 2H), 7.67-7.60 (m, 1H), 7.51 (t, J = 7.6 Hz, 2H), 7.45-7.38 (m, |
| | | | 1H), 7.23 (d, J = 7.6 Hz, 1H), 6.98 (t, J = 8.8 Hz, 1H), 6.53-6.38 |
| | | | (m, 2H), 5.99 (d, J = 7.8 Hz, 1H), 4.50 (br s, 1H), 4.30 (ddd, J = |
| | | | 4.9, 7.3, 11.7 Hz, 1H), 4.01 (br d, J = 12.0 Hz, 2H), 3.75 (br s, |
| | | | 1H), 3.00-2.86 (m, 2H), 2.85-2.69 (m, 3H), 2.61-2.53 (m, 2H), |
| | | | 2.48-2.40 (m, 1H), 2.23 (br t, J = 10.3 Hz, 2H), 2.15-1.92 (m, |
| | | | 5H), 1.86 (dq, J = 4.5, 12.3 Hz, 1H), 1.74 (br d, J = 11.3 Hz, 2H), |
| | | | 1.68-1.52 (m, 4H), 1.51-1.21 (m, 6H) |
| 652 | 807 | 808 | δ = 10.76 (s, 1H), 8.46 (d, J = 3.6 Hz, 1H), 8.19 (s, 1H), 8.05 (br |
| | | | d, J = 7.6 Hz, 1H), 7.97 (s, 1H), 7.74-7.66 (m, 2H), 7.59 (dd, |
| | | | J = 0.9, 7.9 Hz, 1H), 7.54 (ddd, J = 2.1, 6.7, 9.1 Hz, 1H), 7.24 (d, |
| | | | J = 7.8 Hz, 1H), 6.81 (t, J = 9.3 Hz, 1H), 6.54-6.45 (m, 2H), 6.43- |
| | | | 6.30 (m, 2H), 5.78 (d, J = 7.6 Hz, 1H), 4.43 (br s, 1H), 4.31- |
| | | | 4.16 (m, 1H), 3.94 (br s, 4H), 3.70 (br d, J = 1.3 Hz, 1H), 3.23 (s, |
| | | | 4H), 3.07-3.01 (m, 2H), 2.78-2.64 (m, 1H), 2.54 (br s, 2H), |
| | | | 2.12-1.78 (m, 8H), 1.66 (br d, J = 10.1 Hz, 2H), 1.43-1.33 (m, |
| | | | 4H), 1.24 (q, J = 9.7 Hz, 2H) |
| 653 | 806 | 807 | δ 10.78 (s, 1H), 8.52-8.41 (m, 1H), 8.24 (s, 1H), 8.05 (br d, J = |
| | | | 7.8 Hz, 1H), 7.98 (br s, 1H), 7.75-7.66 (m, 2H), 7.60 (dd, J = |
| | | | 1.0, 7.9 Hz, 1H), 7.55 (ddd, J = 2.1, 6.7, 9.1 Hz, 1H), 7.30-7.20 |
| | | | (m, 1H), 6.98 (t, J = 8.9 Hz, 1H), 6.52 (d, J = 9.3 Hz, 1H), 6.47- |
| | | | 6.40 (m, 2H), 6.38-6.33 (m, 1H), 5.99 (d, J = 7.8 Hz, 1H), 4.35- |
| | | | 4.19 (m, 2H), 3.68 (br d, J = 9.0 Hz, 2H), 2.91 (br d, J = 9.3 Hz, |
| | | | 2H), 2.86 (s, 2H), 2.79-2.70 (m, 1H), 2.68 (s, 1H), 2.61-2.56 |
| | | | (m, 1H), 2.56-2.53 (m, 1H), 2.33 (td, J = 1.7, 3.6 Hz, 3H), 2.08 |
| | | | (dt, J = 4.3, 8.5 Hz, 1H), 2.01 (br d, J = 10.9 Hz, 2H), 1.92-1.85 |
| | | | (m, 1H), 1.84-1.76 (m, 2H), 1.75-1.69 (m, 2H), 1.68-1.54 (m, |
| | | | 7H), 1.46 (br d, J = 12.0 Hz, 2H), 1.42-1.31 (m, 5H). |
| 654 | 792 | 793 | δ 11.85 (br d, J = 1.0 Hz, 1H), 10.78 (s, 1H), 8.44 (d, J = 3.6 Hz, |
| | | | 1H), 8.35 (br s, 1H), 8.19 (s, 1H), 7.98-7.80 (m, 2H), 7.71 (dd, |
| | | | J = 2.0, 6.9 Hz, 1H), 7.55 (t, J = 7.8 Hz, 1H), 7.43 (dd, J = 1.9, |
| | | | 6.4 Hz, 1H), 7.28 (br d, J = 7.6 Hz, 1H), 7.09-6.94 (m, 1H), 6.48- |
| | | | 6.40 (m, 2H), 6.33 (t, J = 6.7 Hz, 1H), 5.98 (d, J = 7.8 Hz, 1H), |
| | | | 4.30 (ddd, J = 4.9, 7.3, 11.6 Hz, 2H), 3.93 (br d, J = 11.1 Hz, 2H), |
| | | | 3.20-3.13 (m, 1H), 2.90 (br d, J = 10.9 Hz, 2H), 2.74 (ddd, J = |
| | | | 5.3, 12.2, 17.5 Hz, 2H), 2.57 (br dd, J = 3.8, 13.4 Hz, 3H), 2.14- |
| | | | 2.02 (m, 3H), 2.01-1.88 (m, 4H), 1.87-1.76 (m, 3H), 1.72-1.55 |
| | | | (m, 6H), 1.54-1.19 (m, 5H), 0.94 (br d, J = 12.3 Hz, 2H) |
| 655 | 803 | 804 | δ (DMSO-d6) 10.84-10.72 (m, 1H), 8.39 (d, J = 3.8 Hz, 1H), |
| | | | 7.67-7.47 (m, 2H), 7.39 (br d, J = 5.1 Hz, 2H), 7.13 (br d, J = |
| | | | 7.6 Hz, 2H), 6.82 (t, J = 9.2 Hz, 1H), 6.51 (dd, J = 14.7, 2.2 Hz, |
| | | | 1H), 6.42 (br d, J = 7.9 Hz, 1H), 5.82 (d, J = 7.3 Hz, 1H), 5.10- |
| | | | 4.90 (m, 1H), 4.33-4.19 (m, 1H), 3.83-3.73 (m, 4H), 3.70-3.42 |
| | | | (m, 3H), 3.19-3.14 (m, 4H), 3.05-2.91 (m, 2H), 2.89-2.63 (m, |
| | | | 3H), 2.58 (br d, J = 4.0 Hz, 1H), 2.45-2.17 (m, 4H), 2.15-1.91 |
| | | | (m, 5H), 1.89-1.75 (m, 3H), 1.70-1.47 (m, 5H), 1.40-1.20 (m, |
| | | | 4H) |
| 656 | 794 | 795 | δ (DMSO-d6) 10.78 (s, 1H), 8.45 (d, J = 3.6 Hz, 1H), 8.26 (s, |
| | | | 1H), 8.15 (s, 1H), 7.96 (br d, J = 7.5 Hz, 1H), 7.85 (d, J = 7.8 Hz, |
| | | | 1H), 7.72 (d, J = 7.3 Hz, 2H), 7.68-7.59 (m, 2H), 7.52 (t, J = 7.6 |
| | | | Hz, 2H), 7.46-7.40 (m, 1H), 7.21 (d, J = 7.8 Hz, 1H), 6.82 (t, |
| | | | J = 9.3 Hz, 1H), 6.51 (dd, J = 15.1, 2.2 Hz, 1H), 6.42 (br d, J = 8.5 |
| | | | Hz, 1H), 5.83 (d, J = 7.9 Hz, 1H), 5.11-4.92 (m, 1H), 4.31-4.21 |
| | | | (m, 1H), 3.71-3.61 (m, 1H), 3.57-3.46 (m, 1H), 3.22 (br d, J = |
| | | | 10.9 Hz, 1H), 3.07-2.95 (m, 2H), 2.84-2.65 (m, 3H), 2.62-2.53 |
| | | | (m, 2H), 2.34-2.22 (m, 2H), 2.13-2.06 (m, 2H), 2.00 (br dd, |
| | | | J = 11.7, 7.9 Hz, 3H), 1.89-1.74 (m, 3H), 1.71-1.52 (m, 5H), 1.43- |
| | | | 1.29 (m, 3H), 1.24 (br d, J = 8.9 Hz, 2H) |
| 657 | 784 | 785 | δ (DMSO-d6) 10.81 (br s, 1H), 8.40 (d, J = 3.7 Hz, 1H), 7.58 (br |
| | | | d, J = 7.6 Hz, 1H), 7.41 (br d, J = 6.5 Hz, 2H), 7.18-7.12 (m, |
| | | | 3H), 7.00 (s, 1H), 6.48-6.42 (m, 2H), 6.00 (d, J = 6.7 Hz, 1H), |
| | | | 4.35-4.27 (m, 1H), 3.78 (br s, 4H), 3.67-3.60 (m, 1H), 3.48- |
| | | | 3.40 (m, 2H), 3.18 (br d, J = 4.4 Hz, 4H), 2.92-2.88 (m, 2H), |
| | | | 2.76-2.69 (m, 1H), 2.29-2.24 (m, 3H), 2.07-2.01 (m, 3H), 1.83- |
| | | | 1.73 (m, 8H), 1.67-1.57 (m, 5H), 1.46 (br d, J = 11.6 Hz, 2H), |
| | | | 1.31 (br d, J = 13.0 Hz, 4H), 1.19-1.13 (m, 2H) |
| 658 | 775 | 776 | δ (DMSO-d6) 10.80 (s, 1H), 8.45 (d, J = 3.8 Hz, 1H), 8.27 (br s, |
| | | | 1H), 8.16 (s, 1H), 7.97 (br d, J = 6.8 Hz, 1H), 7.85 (d, J = 7.8 Hz, |
| | | | 1H), 7.75-7.69 (m, 2H), 7.68-7.58 (m, 2H), 7.54-7.49 (m, 2H), |
| | | | 7.45-7.40 (m, 1H), 7.20 (d, J = 7.8 Hz, 1H), 7.01-6.94 (m, 1H), |
| | | | 6.51-6.41 (m, 2H), 6.03 (d, J = 7.6 Hz, 1H), 4.36-4.26 (m, 1H), |
| | | | 3.72-3.62 (m, 1H), 3.50-3.42 (m, 2H), 3.12-3.02 (m, 2H), 2.80- |
| | | | 2.64 (m, 2H), 2.59 (br d, J = 3.8 Hz, 1H), 2.11-2.00 (m, 4H), |
| | | | 1.90-1.81 (m, 5H), 1.78-1.66 (m, 6H), 1.53-1.11 (m, 10H) |
| 659 | 784 | 785 | δ (DMSO-d6) 10.84-10.73 (m, 1H), 8.41-8.37 (m, 1H), 7.62- |
| | | | 7.51 (m, 2H), 7.44-7.35 (m, 2H), 7.19-7.09 (m, 2H), 7.03-6.94 |
| | | | (m, 1H), 6.48-6.39 (m, 2H), 6.01-5.96 (m, 1H), 4.35-4.26 (m, |
| | | | 1H), 3.77 (br d, J = 4.3 Hz, 4H), 3.67-3.59 (m, 2H), 3.55-3.45 |
| | | | (m, 3H), 3.17 (br s, 4H), 2.89 (br d, J = 10.8 Hz, 2H), 2.27 (br t, |
| | | | J = 10.3 Hz, 3H), 2.12-2.06 (m, 1H), 2.02-1.94 (m, 3H), 1.86- |
| | | | 1.72 (m, 7H), 1.69-1.55 (m, 4H), 1.41-1.31 (m, 4H), 1.30-1.21 |
| | | | (m, 4H) |
| 660 | 775 | 776 | δ (DMSO-d6) 10.82-10.77 (m, 1H), 8.45 (d, J = 3.8 Hz, 1H), |
| | | | 8.28-8.22 (m, 1H), 8.17 (s, 1H), 7.96 (br d, J = 7.5 Hz, 1H), 7.85 |
| | | | (br d, J = 7.5 Hz, 1H), 7.75-7.69 (m, 2H), 7.68-7.59 (m, 2H), |
| | | | 7.52 (t, J = 7.6 Hz, 2H), 7.46-7.39 (m, 1H), 7.20 (d, J = 7.8 Hz, |
| | | | 1H), 7.02-6.94 (m, 1H), 6.50-6.41 (m, 2H), 6.03 (br d, J = 7.6 |
| | | | Hz, 1H), 4.38-4.22 (m, 1H), 3.72-3.59 (m, 2H), 3.51-3.49 (m, |
| | | | 1H), 3.08-3.02 (m, 2H), 2.79-2.65 (m, 2H), 2.59 (br d, J = 3.9 |
| | | | Hz, 1H), 2.13-2.05 (m, 1H), 2.04-1.95 (m, 3H), 1.94-1.85 (m, |
| | | | 3H), 1.78 (br d, J = 9.6 Hz, 5H), 1.71 (br s, 4H), 1.47-1.22 (m, |
| | | | 9H) |
| 661 | 785 | 786 | δ (DMSO-d6) 10.78 (s, 1H), 8.39 (d, J = 3.8 Hz, 1H), 8.15 (s, |
| | | | 1H), 7.63-7.52 (m, 2H), 7.46-7.34 (m, 2H), 7.17-7.09 (m, 2H), |
| | | | 6.82 (t, J = 9.3 Hz, 1H), 6.51 (dd, J = 15.1, 2.4 Hz, 1H), 6.42 (dd, |
| | | | J = 8.8, 1.9 Hz, 1H), 5.80 (s, 1H), 4.31-4.20 (m, 1H), 3.80-3.75 |
| | | | (m, 4H), 3.68-3.58 (m, 1H), 3.48-3.43 (m, 1H), 3.18-3.15 (m, |
| | | | 4H), 2.86 (br s, 4H), 2.79-2.73 (m, 1H), 2.70-2.66 (m, 3H), |
| | | | 2.59 (br t, J = 3.9 Hz, 1H), 2.39-2.27 (m, 1H), 2.13-1.99 (m, |
| | | | 4H), 1.88-1.79 (m, 5H), 1.74 (br d, J = 11.6 Hz, 2H), 1.51-1.40 |
| | | | (m, 2H), 1.29 (br t, J = 12.8 Hz, 4H), 1.23-1.10 (m, 3H) |
| 662 | 776 | 777 | δ (DMSO-d6) 10.77 (s, 1H), 8.44 (d, J = 3.8 Hz, 1H), 8.26 (br s, |
| | | | 1H), 8.13 (s, 1H), 7.96 (br d, J = 6.9 Hz, 1H), 7.84 (d, J = 7.9 Hz, |
| | | | 1H), 7.71 (d, J = 7.3 Hz, 2H), 7.64 (t, J = 7.8 Hz, 1H), 7.59 (br d, |
| | | | J = 7.8 Hz, 1H), 7.53-7.48 (m, 2H), 7.44-7.39 (m, 1H), 7.18 (d, |
| | | | J = 7.8 Hz, 1H), 6.83 (t, J = 9.4 Hz, 1H), 6.51 (dd, J = 15.1, 2.2 |
| | | | Hz, 1H), 6.42 (dd, J = 8.8, 1.9 Hz, 1H), 5.82 (br d, J = 7.4 Hz, |
| | | | 1H), 4.25 (ddd, J = 11.6, 7.2, 4.8 Hz, 1H), 3.72-3.61 (m, 1H), |
| | | | 3.55-3.40 (m, 2H), 2.90 (br s, 4H), 2.82-2.66 (m, 4H), 2.59 (br |
| | | | t, J = 3.9 Hz, 1H), 2.56-2.53 (m, 1H), 2.11-1.99 (m, 4H), 1.86 |
| | | | (dt, J = 12.2, 4.6 Hz, 5H), 1.74 (br d, J = 11.8 Hz, 2H), 1.52-1.41 |
| | | | (m, 2H), 1.37-1.25 (m, 4H), 1.23-1.13 (m, 2H) |
| 663 | 852 | 853 | δ (DMSO-d6) 11.01-10.56 (m, 1H), 8.47 (d, J = 3.6 Hz, 1H), |
| | | | 8.06 (br d, J = 7.5 Hz, 1H), 7.98 (s, 1H), 7.74-7.67 (m, 2H), 7.60 |
| | | | (dd, J = 1.0, 7.9 Hz, 1H), 7.55 (ddd, J = 2.1, 6.8, 9.1 Hz, 1H), 7.26 |
| | | | (d, J = 7.8 Hz, 1H), 7.01-6.94 (m, 1H), 6.52 (d, J = 9.0 Hz, 1H), |
| | | | 6.48-6.43 (m, 2H), 6.36 (dt, J = 1.2, 6.7 Hz, 1H), 6.03 (d, J = 7.6 |
| | | | Hz, 1H), 5.15-5.07 (m, 1H), 4.62-4.45 (m, 2H), 4.30 (ddd, J = |
| | | | 4.9, 7.3, 11.8 Hz, 1H), 4.10 (br d, J = 12.5 Hz, 1H), 3.78-3.65 |
| | | | (m, 3H), 3.18-3.04 (m, 3H), 2.95-2.85 (m, 1H), 2.79-2.67 (m, |
| | | | 1H), 2.65-2.56 (m, 2H), 2.12-2.05 (m, 1H), 1.96 (br d, J = 6.4 |
| | | | Hz, 4H), 1.92-1.80 (m, 1H), 1.74-1.67 (m, 2H), 1.62-1.48 (m, |
| | | | 6H), 1.48-1.32 (m, 6H) |
| 664 | 778 | 779 | δ 10.78 (s, 1H), 8.46 (d, J = 3.6 Hz, 1H), 8.31-8.18 (m, 1H), 7.97 |
| | | | (br d, J = 6.9 Hz, 1H), 7.85 (s, 1H), 7.78-7.59 (m, 3H), 7.57- |
| | | | 7.48 (m, 2H), 7.46-7.38 (m, 1H), 7.23 (d, J = 7.6 Hz, 1H), 6.82 |
| | | | (t, J = 9.4 Hz, 1H), 6.57-6.36 (m, 2H), 5.80 (d, J = 7.6 Hz, 1H), |
| | | | 4.50 (br s, 1H), 4.26 (ddd, J = 4.9, 7.3, 11.7 Hz, 1H), 4.00 (br d, |
| | | | J = 12.5 Hz, 2H), 3.84-3.69 (m, 1H), 2.84 (br s, 5H), 2.73 (dt, |
| | | | J = 5.9, 11.9 Hz, 2H), 2.65-2.56 (m, 5H), 2.46-2.34 (m, 1H), 2.13- |
| | | | 1.91 (m, 5H), 1.90-1.72 (m, 3H), 1.54-1.39 (m, 4H), 1.38- |
| | | | 1.23 (m, 2H) |
| 665 | 800 | 801 | δ 10.80 (s, 1H), 8.40 (d, J = 3.7 Hz, 1H), 7.55 (br s, 1H), 7.44- |
| | | | 7.32 (m, 2H), 7.27-7.10 (m, 2H), 7.06-6.90 (m, 1H), 6.56-6.41 |
| | | | (m, 2H), 6.08 (br d, J = 7.0 Hz, 1H), 4.48-4.24 (m, 1H), 4.17- |
| | | | 4.03 (m, 1H), 3.85-3.70 (m, 7H), 3.40 (br s, 1H), 3.23-3.10 (m, |
| | | | 6H), 3.06-2.92 (m, 2H), 2.82-2.67 (m, 2H), 2.65-2.54 (m, 2H), |
| | | | 2.18-1.95 (m, 5H), 1.94-1.81 (m, 2H), 1.80-1.64 (m, 6H), 1.60- |
| | | | 1.28 (m, 8H) |
| 666 | 806 | 807 | δ 10.78 (s, 1H), 8.46 (d, J = 3.5 Hz, 1H), 8.16 (s, 1H), 8.05 (br d, |
| | | | J = 7.5 Hz, 1H), 7.97 (s, 1H), 7.77-7.64 (m, 2H), 7.62-7.49 (m, |
| | | | 2H), 7.24 (br d, J = 7.6 Hz, 1H), 6.97 (t, J = 8.8 Hz, 1H), 6.51 (d, |
| | | | J = 9.1 Hz, 1H), 6.48-6.40 (m, 2H), 6.35 (t, J = 6.7 Hz, 1H), 6.00 |
| | | | (br d, J = 7.8 Hz, 1H), 4.43 (br s, 1H), 4.34-4.24 (m, 1H), 3.90 |
| | | | (br s, 2H), 3.81-3.68 (m, 4H), 2.86 (br d, J = 10.5 Hz, 2H), 2.73 |
| | | | (ddd, J = 5.3, 12.2, 17.6 Hz, 1H), 2.64-2.52 (m, 3H), 2.34-2.22 |
| | | | (m, 2H), 2.08 (td, J = 4.1, 8.6 Hz, 1H), 2.00-1.87 (m, 6H), 1.84 |
| | | | (br s, 2H), 1.68-1.50 (m, 4H), 1.37 (br t, J = 9.1 Hz, 4H) |
| 667 | 799 | 800 | δ 10.77 (s, 1H), 8.45 (d, J = 3.6 Hz, 1H), 7.96 (s, 1H), 7.84 (br d, |
| | | | J = 6.5 Hz, 1H), 7.62-7.50 (m, 2H), 7.32 (br d, J = 7.6 Hz, 1H), |
| | | | 6.82 (t, J = 9.3 Hz, 1H), 6.50 (dd, J = 2.4, 15.0 Hz, 1H), 6.41 (dd, |
| | | | J = 2.2, 8.7 Hz, 1H), 5.79 (d, J = 7.6 Hz, 1H), 4.37 (t, J = 5.3 Hz, |
| | | | 2H), 4.33-4.20 (m, 2H), 3.93 (br d, J = 11.0 Hz, 2H), 3.71 (t, |
| | | | J = 6.1 Hz, 2H), 3.22-3.08 (m, 1H), 2.90-2.79 (m, 4H), 2.78- |
| | | | 2.65 (m, 2H), 2.61-2.52 (m, 2H), 2.44 (br s, 3H), 2.18-2.05 (m, |
| | | | 5H), 2.00-1.84 (m, 3H), 1.80 (br d, J = 12.5 Hz, 2H), 1.66 (br d, |
| | | | J = 12.3 Hz, 2H), 1.56-1.20 (m, 6H), 0.94 (q, J = 12.0 Hz, 2H) |
| 668 | 813 | 814 | δ = 11.46-11.31 (m, 1H), 10.79 (s, 1H), 8.47 (d, J = 3.6 Hz, 1H), |
| | | | 8.05 (d, J = 7.5 Hz, 1H), 7.97 (s, 1H), 7.73-7.67 (m, 2H), 7.62- |
| | | | 7.58 (m, 1H), 7.55 (ddd, J = 2.1, 6.7, 9.1 Hz, 1H), 7.46-7.06 (m, |
| | | | 1H), 6.87 (t, J = 9.4 Hz, 1H), 6.60-6.44 (m, 3H), 6.35 (dt, J = |
| | | | 1.3, 6.7 Hz, 1H), 5.55-5.31 (m, 1H), 4.55 (br s, 1H), 4.29 (br dd, |
| | | | J = 4.7, 11.4 Hz, 2H), 3.78-3.65 (m, 2H), 3.63-3.39 (m, 6H), |
| | | | 3.29 (br d, J = 11.2 Hz, 1H), 3.18 (br d, J = 6.7 Hz, 2H), 3.01- |
| | | | 2.83 (m, 1H), 2.73 (ddd, J = 5.3, 12.0, 17.6 Hz, 2H), 2.61-2.53 |
| | | | (m, 1H), 2.19-2.04 (m, 3H), 1.99 (br d, J = 9.9 Hz, 4H), 1.86 (br |
| | | | dd, J = 4.4, 12.1 Hz, 1H), 1.56-1.32 (m, 4H) |
| 669 | 806 | 807 | δ (DMSO-d6) 10.79 (s, 1H), 8.47 (d, J = 3.5 Hz, 1H), 8.07 (br d, |
| | | | J = 7.5 Hz, 1H), 7.98 (s, 1H), 7.74-7.70 (m, 2H), 7.60 (dd, J = |
| | | | 7.9, 1.0 Hz, 1H), 7.57-7.51 (m, 1H), 7.29 (br d, J = 7.6 Hz, 1H), |
| | | | 6.98 (br t, J = 8.5 Hz, 1H), 6.52 (d, J = 9.3 Hz, 1H), 6.48-6.43 |
| | | | (m, 2H), 6.36 (dt, J = 6.7, 1.2 Hz, 1H), 6.03 (d, J = 7.8 Hz, 1H), |
| | | | 4.53 (br d, J = 12.1 Hz, 1H), 4.43-4.36 (m, 1H), 4.34-4.28 (m, |
| | | | 1H), 4.11 (br d, J = 10.8 Hz, 1H), 3.97 (br d, J = 12.0 Hz, 1H), |
| | | | 3.70-3.59 (m, 1H), 3.16-3.08 (m, 2H), 2.97-2.87 (m, 2H), 2.79- |
| | | | 2.68 (m, 1H), 2.58 (td, J = 17.4, 4.1 Hz, 4H), 2.13-2.06 (m, |
| | | | 1H), 2.03-1.97 (m, 2H), 1.93-1.83 (m, 1H), 1.69 (br d, J = 8.3 |
| | | | Hz, 6H), 1.51-1.43 (m, 4H), 1.40-1.29 (m, 4H) |
| 670 | 785 | 786 | δ (DMSO-d6) 10.78 (s, 1H), 8.41 (d, J = 3.6 Hz, 1H), 7.52 (br s, |
| | | | 1H), 7.39 (br d, J = 5.4 Hz, 2H), 7.26 (d, J = 7.9 Hz, 1H), 7.15 (br |
| | | | d, J = 5.9 Hz, 1H), 6.83 (t, J = 9.3 Hz, 1H), 6.51 (br d, J = 15.0 |
| | | | Hz, 1H), 6.42 (br d, J = 9.0 Hz, 1H), 5.80 (br d, J = 7.6 Hz, 1H), |
| | | | 4.35-4.24 (m, 2H), 3.94 (br d, J = 9.8 Hz, 2H), 3.80-3.75 (m, |
| | | | 4H), 3.20-3.13 (m, 6H), 2.85 (br s, 4H), 2.79-2.66 (m, 3H), |
| | | | 2.59 (br s, 1H), 2.47-2.41 (m, 3H), 2.12 (br d, J = 7.4 Hz, 3H), |
| | | | 1.97 (br s, 1H), 1.87-1.77 (m, 3H), 1.67 (br d, J = 10.6 Hz, 2H), |
| | | | 1.50 (br s, 1H), 1.40 (br s, 2H), 1.33 (br s, 2H), 1.02-0.91 (m, |
| | | | 3H) |
| 671 | 784 | 785 | δ (DMSO-d6) 10.80 (s, 1H), 8.42 (d, J = 3.8 Hz, 1H), 8.18 (s, |
| | | | 1H), 7.54 (br s, 1H), 7.40 (br d, J = 5.6 Hz, 2H), 7.27 (d, J = 7.7 |
| | | | Hz, 1H), 7.17 (br s, 1H), 7.00 (s, 1H), 6.47 (br dd, J = 4.5, 2.1 Hz, |
| | | | 2H), 6.01 (d, J = 7.9 Hz, 1H), 4.35-4.27 (m, 2H), 3.99-3.91 (m, |
| | | | 2H), 3.81-3.76 (m, 5H), 3.19-3.16 (m, 5H), 2.94 (br d, J = 10.3 |
| | | | Hz, 2H), 2.75 (br s, 2H), 2.60 (br d, J = 4.2 Hz, 2H), 2.14 (br d, |
| | | | J = 7.1 Hz, 3H), 2.00-1.93 (m, 3H), 1.92-1.78 (m, 4H), 1.66 (br |
| | | | s, 5H), 1.52-1.31 (m, 6H), 1.02-0.91 (m, 2H) |
| 672 | 784 | 785 | δ (DMSO-d6) 10.79 (s, 1H), 8.39 (d, J = 3.8 Hz, 1H), 7.59-7.49 |
| | | | (m, 1H), 7.44-7.35 (m, 2H), 7.22-7.12 (m, 2H), 7.02-6.94 (m, |
| | | | 1H), 6.49-6.38 (m, 2H), 6.04-5.95 (m, 1H), 4.39 (br d, J = 12.2 |
| | | | Hz, 1H), 4.35-4.26 (m, 1H), 3.98-3.89 (m, 1H), 3.80-3.74 (m, |
| | | | 4H), 3.64 (td, J = 3.1, 7.3 Hz, 1H), 3.17 (br d, J = 4.8 Hz, 4H), |
| | | | 3.07-2.82 (m, 4H), 2.77-2.65 (m, 2H), 2.18-1.88 (m, 9H), 1.83- |
| | | | 1.69 (m, 6H), 1.63 (br s, 4H), 1.51-1.42 (m, 2H), 1.39-1.31 |
| | | | (m, 2H), 1.08-0.94 (m, 1H), 0.93-0.83 (m, 1H) |
| 673 | 787 | 788 | δ (DMSO-d6) 10.78 (s, 1H), 8.40 (br d, J = 2.9 Hz, 1H), 7.54 (br |
| | | | s, 1H), 7.43-7.35 (m, 2H), 7.19-7.12 (m, 2H), 6.82 (br t, J = 9.0 |
| | | | Hz, 1H), 6.51 (br d, J = 15.4 Hz, 1H), 6.42 (br d, J = 7.5 Hz, 1H), |
| | | | 5.80 (br d, J = 7.7 Hz, 1H), 4.55-4.45 (m, 1H), 4.30-4.21 (m, |
| | | | 1H), 4.05-3.95 (m, 2H), 3.77 (br d, J = 4.3 Hz, 4H), 3.74-3.68 |
| | | | (m, 1H), 3.17 (br s, 4H), 2.84 (br s, 4H), 2.80-2.67 (m, 3H), 2.61 |
| | | | (br s, 5H), 2.47-2.37 (m, 2H), 2.12-2.05 (m, 1H), 1.98 (br d, |
| | | | J = 7.2 Hz, 4H), 1.88-1.82 (m, 1H), 1.78 (br d, J = 11.2 Hz, 2H), |
| | | | 1.42 (br s, 4H), 1.30 (br d, J = 9.9 Hz, 1H) |
| 674 | 801 | 802 | δ (DMSO-d6) 10.78 (s, 1H), 8.45 (d, J = 3.5 Hz, 1H), 8.18 (s, |
| | | | 1H), 7.96 (s, 1H), 7.87-7.82 (m, 1H), 7.59-7.49 (m, 2H), 7.22 |
| | | | (br d, J = 7.6 Hz, 1H), 6.82 (t, J = 9.3 Hz, 1H), 6.51 (dd, J = 15.3, |
| | | | 2.2 Hz, 1H), 6.44-6.39 (m, 1H), 5.80 (d, J = 7.6 Hz, 1H), 4.50 |
| | | | (br d, J = 4.0 Hz, 1H), 4.37 (t, J = 5.3 Hz, 2H), 4.25 (ddd, J = |
| | | | 11.5, 7.3, 4.5 Hz, 1H), 4.00 (br d, J = 12.1 Hz, 2H), 3.72 (br t, |
| | | | J = 6.1 Hz, 3H), 2.84 (br s, 4H), 2.79-2.66 (m, 3H), 2.61 (br s, |
| | | | 4H), 2.47-2.35 (m, 2H), 2.16-2.07 (m, 3H), 1.97 (br d, J = 9.4 |
| | | | Hz, 4H), 1.89-1.75 (m, 3H), 1.48-1.38 (m, 4H), 1.35-1.26 (m, |
| | | | 2H) |
| 675 | 799 | 800 | δ (DMSO-d6) 10.78 (s, 1H), 8.44 (d, J = 3.6 Hz, 1H), 7.91-7.80 |
| | | | (m, 2H), 7.55 (t, J = 7.8 Hz, 1H), 7.45 (br d, J = 8.0 Hz, 1H), 7.22 |
| | | | (br d, J = 7.7 Hz, 1H), 6.82 (t, J = 9.3 Hz, 1H), 6.51 (dd, J = 2.2, |
| | | | 15.1 Hz, 1H), 6.42 (dd, J = 2.0, 8.5 Hz, 1H), 5.80 (d, J = 7.6 Hz, |
| | | | 1H), 4.50 (br s, 1H), 4.32-4.20 (m, 1H), 4.00 (br d, J = 12.5 Hz, |
| | | | 2H), 3.79-3.60 (m, 3H), 2.84 (br s, 4H), 2.81-2.65 (m, 3H), |
| | | | 2.60 (br d, J = 3.7 Hz, 4H), 2.54 (br d, J = 4.2 Hz, 1H), 2.42 (br t, |
| | | | J = 6.2 Hz, 3H), 2.13-2.06 (m, 1H), 2.03-1.93 (m, 4H), 1.93- |
| | | | 1.83 (m, 5H), 1.78 (br d, J = 12.8 Hz, 2H), 1.42 (q, J = 8.0 Hz, |
| | | | 4H), 1.36-1.26 (m, 2H) |
| 676 | 786 | 787 | δ (DMSO-d6) 10.77 (s, 1H), 8.39 (d, J = 3.8 Hz, 1H), 8.24 (s, |
| | | | 1H), 7.53 (br s, 1H), 7.42-7.32 (m, 2H), 7.13 (br d, J = 7.3 Hz, |
| | | | 2H), 6.98 (t, J = 8.8 Hz, 1H), 6.48-6.39 (m, 2H), 5.97 (d, J = 7.5 |
| | | | Hz, 1H), 4.54-4.41 (m, 1H), 4.35-4.24 (m, 1H), 4.01 (br d, J = |
| | | | 12.4 Hz, 2H), 3.78-3.74 (m, 4H), 3.73-3.66 (m, 1H), 3.19-3.12 |
| | | | (m, 5H), 2.91 (br d, J = 9.9 Hz, 2H), 2.81-2.65 (m, 3H), 2.58 (br |
| | | | d, J = 4.0 Hz, 1H), 2.44 (br s, 1H), 2.28-2.18 (m, 2H), 2.12- |
| | | | 2.05 (m, 1H), 2.03-1.92 (m, 4H), 1.89-1.80 (m, 1H), 1.77-1.70 |
| | | | (m, 2H), 1.68-1.55 (m, 4H), 1.48-1.38 (m, 4H), 1.36-1.27 (m, |
| | | | 2H) |
| 677 | 800 | 801 | δ (DMSO-d6) 10.77 (s, 1H), 8.44 (d, J = 3.5 Hz, 1H), 8.18 (s, |
| | | | 1H), 7.95 (s, 1H), 7.84 (br d, J = 6.5 Hz, 1H), 7.60-7.48 (m, 2H), |
| | | | 7.20 (d, J = 7.8 Hz, 1H), 7.01-6.92 (m, 1H), 6.48-6.38 (m, 2H), |
| | | | 5.97 (d, J = 7.6 Hz, 1H), 4.54-4.43 (m, 1H), 4.37 (t, J = 5.4 Hz, |
| | | | 2H), 4.33-4.24 (m, 1H), 4.05-3.94 (m, 2H), 3.71 (br t, J = 6.1 |
| | | | Hz, 3H), 2.92 (br d, J = 10.6 Hz, 2H), 2.80-2.66 (m, 3H), 2.62- |
| | | | 2.54 (m, 2H), 2.45 (br s, 1H), 2.24 (br t, J = 10.4 Hz, 2H), 2.15- |
| | | | 2.04 (m, 3H), 2.01-1.92 (m, 4H), 1.91-1.82 (m, 1H), 1.77-1.70 |
| | | | (m, 2H), 1.69-1.54 (m, 4H), 1.48-1.37 (m, 4H), 1.36-1.27 (m, |
| | | | 2H) |
| 678 | 798 | 799 | δ (DMSO-d6) 10.78 (s, 1H), 8.43 (d, J = 3.6 Hz, 1H), 8.23 (s, |
| | | | 1H), 7.87 (s, 1H), 7.84 (br d, J = 7.6 Hz, 1H), 7.57-7.52 (m, 1H), |
| | | | 7.45 (dd, J = 8.0, 0.8 Hz, 1H), 7.21 (d, J = 7.6 Hz, 1H), 7.01- |
| | | | 6.95 (m, 1H), 6.45-6.41 (m, 1H), 5.98 (d, J = 7.6 Hz, 1H), 5.57 |
| | | | (d, J = 8.0 Hz, 1H), 4.48 (br s, 1H), 4.29 (ddd, J = 11.8, 7.2, 4.9 |
| | | | Hz, 1H), 4.01 (br d, J = 12.8 Hz, 2H), 3.72 (br dd, J = 3.6, 2.4 Hz, |
| | | | 1H), 3.64 (t, J = 5.5 Hz, 2H), 2.91 (br d, J = 10.3 Hz, 2H), 2.73 |
| | | | (ddd, J = 17.6, 12.1, 5.3 Hz, 2H), 2.61-2.53 (m, 2H), 2.44-2.40 |
| | | | (m, 2H), 2.27-2.19 (m, 2H), 2.11-2.04 (m, 1H), 1.97 (br d, J = |
| | | | 8.9 Hz, 3H), 1.90-1.84 (m, 4H), 1.77-1.69 (m, 4H), 1.61 (br d, |
| | | | J = 13.9 Hz, 4H), 1.54-1.47 (m, 1H), 1.41 (br d, J = 7.5 Hz, 2H), |
| | | | 1.33-1.21 (m, 3H), 1.15-1.02 (m, 2H) |
| 679 | 829 | 830 | δ (DMSO-d6) 10.90-10.83 (m, 1H), 8.47 (d, J = 3.6 Hz, 1H), |
| | | | 8.05 (br d, J = 7.5 Hz, 1H), 7.97 (s, 1H), 7.74-7.66 (m, 2H), 7.59 |
| | | | (br d, J = 8.5 Hz, 2H), 7.57-7.51 (m, 1H), 7.44 (s, 1H), 7.33 (br |
| | | | d, J = 7.5 Hz, 1H), 7.03 (br d, J = 8.5 Hz, 1H), 6.51 (d, J = 9.3 |
| | | | Hz, 1H), 6.35 (t, J = 6.6 Hz, 1H), 4.49-4.41 (m, 1H), 4.32 (dd, |
| | | | J = 9.7, 5.1 Hz, 1H), 3.96 (s, 3H), 3.92 (br d, J = 12.6 Hz, 2H), 3.19- |
| | | | 3.13 (m, 1H), 2.94 (br d, J = 10.0 Hz, 3H), 2.71-2.55 (m, 3H), |
| | | | 2.39-2.29 (m, 1H), 2.21-2.16 (m, 1H), 2.13 (br d, J = 6.8 Hz, |
| | | | 2H), 2.02-1.84 (m, 6H), 1.82-1.72 (m, 4H), 1.61-1.47 (m, 2H), |
| | | | 1.43-1.25 (m, 5H), 1.03-0.90 (m, 3H) |
| 680 | 808 | 809 | δ (DMSO-d6) 10.79 (s, 1H), 9.28 (br s, 1H), 8.49 (d, J = 3.6 Hz, |
| | | | 1H), 8.06 (d, J = 7.1 Hz, 1H), 7.99 (s, 1H), 7.75-7.67 (m, 2H), |
| | | | 7.64-7.59 (m, 1H), 7.57-7.51 (m, 1H), 7.43-7.27 (m, 1H), 6.97 |
| | | | (t, J = 8.6 Hz, 1H), 6.55-6.45 (m, 3H), 6.36 (dt, J = 6.7, 1.2 Hz, |
| | | | 1H), 4.33 (br dd, J = 11.5, 4.8 Hz, 2H), 4.01-3.87 (m, 2H), 3.62 |
| | | | (br d, J = 11.3 Hz, 2H), 3.32 (br s, 1H), 3.20 (br d, J = 11.4 Hz, |
| | | | 3H), 3.05 (br d, J = 3.3 Hz, 2H), 2.94 (br t, J = 12.3 Hz, 1H), 2.74 |
| | | | (ddd, J = 17.6, 12.1, 5.4 Hz, 2H), 2.62-2.54 (m, 2H), 2.35-2.18 |
| | | | (m, 2H), 2.13-2.05 (m, 1H), 1.97 (br d, J = 10.9 Hz, 1H), 1.93- |
| | | | 1.72 (m, 9H), 1.49-1.42 (m, 4H), 1.35-1.23 (m, 1H) |
| 681 | 807 | 808 | δ 10.87-10.68 (m, 1H), 8.46 (d, J = 3.6 Hz, 1H), 8.05 (br d, J = |
| | | | 7.5 Hz, 1H), 7.97 (s, 1H), 7.77-7.50 (m, 4H), 7.25 (d, J = 7.5 |
| | | | Hz, 1H), 6.81 (t, J = 9.3 Hz, 1H), 6.56-6.45 (m, 2H), 6.41 (dd, |
| | | | J = 2.0, 8.8 Hz, 1H), 6.38-6.31 (m, 1H), 5.80 (d, J = 7.6 Hz, 1H), |
| | | | 4.42 (br s, 1H), 4.24 (ddd, J = 4.9, 7.2, 11.7 Hz, 1H), 3.91 (br s, |
| | | | 2H), 3.83-3.62 (m, 3H), 2.82 (br s, 4H), 2.77-2.55 (m, 3H), |
| | | | 2.43-2.19 (m, 6H), 2.07 (dt, J = 4.3, 8.7 Hz, 1H), 2.03-1.75 (m, |
| | | | 7H), 1.46-1.30 (m, 4H) |
| 682 | 782 | 783 | δ 10.78 (s, 1H), 8.39 (d, J = 3.8 Hz, 1H), 8.29 (s, 1H), 7.52 (br s, |
| | | | 1H), 7.33 (br d, J = 6.1 Hz, 2H), 7.24 (d, J = 7.5 Hz, 1H), 7.13- |
| | | | 7.08 (m, 1H), 6.99 (t, J = 8.8 Hz, 1H), 6.48-6.39 (m, 2H), 5.98 |
| | | | (d, J = 7.6 Hz, 1H), 4.31 (br dd, J = 4.5, 7.4 Hz, 2H), 3.93 (br dd, |
| | | | J = 3.4, 7.1 Hz, 2H), 3.22-3.16 (m, 6H), 2.89 (br d, J = 10.8 Hz, |
| | | | 2H), 2.72 (br d, J = 12.0 Hz, 2H), 2.61-2.53 (m, 3H), 2.12-2.04 |
| | | | (m, 3H), 2.01-1.86 (m, 5H), 1.85-1.76 (m, 2H), 1.69-1.60 (m, |
| | | | 9H), 1.58-1.53 (m, 2H), 1.50-1.26 (m, 5H), 0.94 (br d, J = 12.0 |
| | | | Hz, 2H). |
| 683 | 798 | 799 | δ 10.79 (s, 1H), 9.86 (br s, 1H), 8.46 (d, J = 3.5 Hz, 1H), 8.00 (s, |
| | | | 1H), 7.86 (br d, J = 5.5 Hz, 1H), 7.69-7.50 (m, 2H), 7.44-7.22 |
| | | | (m, 1H), 6.96 (t, J = 8.5 Hz, 1H), 6.59-6.37 (m, 2H), 4.32 (br dd, |
| | | | J = 4.7, 11.4 Hz, 2H), 4.23 (s, 2H), 4.03-3.98 (m, 3H), 3.94 (br |
| | | | d, J = 12.3 Hz, 2H), 3.54 (br d, J = 11.5 Hz, 2H), 3.27 (br d, J = |
| | | | 5.1 Hz, 1H), 3.17 (br t, J = 11.4 Hz, 1H), 3.08-2.97 (m, 2H), |
| | | | 2.96-2.83 (m, 3H), 2.80-2.69 (m, 2H), 2.64-2.55 (m, 2H), 2.20- |
| | | | 2.04 (m, 3H), 2.02-1.95 (m, 1H), 1.95-1.74 (m, 7H), 1.68 (br |
| | | | d, J = 11.9 Hz, 2H), 1.54-1.36 (m, 3H), 1.35-1.25 (m, 1H), 1.19- |
| | | | 0.99 (m, 2H) |
| 684 | 798 | 799 | δ 10.78 (br d, J = 1.1 Hz, 1H), 8.45 (d, J = 3.6 Hz, 1H), 7.96 (s, |
| | | | 1H), 7.84 (br d, J = 6.6 Hz, 1H), 7.60-7.51 (m, 2H), 7.32 (br d, |
| | | | J = 7.9 Hz, 1H), 7.02-6.95 (m, 1H), 6.46-6.39 (m, 2H), 5.98 (d, |
| | | | J = 7.8 Hz, 1H), 4.37 (t, J = 5.4 Hz, 2H), 4.29 (br d, J = 3.9 Hz, |
| | | | 2H), 3.99-3.90 (m, 2H), 3.71 (t, J = 6.0 Hz, 2H), 3.15 (br s, 1H), |
| | | | 2.89 (br d, J = 11.1 Hz, 2H), 2.78-2.66 (m, 2H), 2.56 (br dd, J = |
| | | | 3.9, 13.3 Hz, 3H), 2.16-2.05 (m, 5H), 1.99-1.86 (m, 5H), 1.84- |
| | | | 1.76 (m, 2H), 1.68 (br s, 1H), 1.63 (br d, J = 8.1 Hz, 5H), 1.50- |
| | | | 1.28 (m, 5H), 0.93 (br d, J = 12.8 Hz, 2H). |
| 685 | 796 | 797 | δ 10.78 (s, 1H), 8.44 (d, J = 3.6 Hz, 1H), 8.19 (s, 1H), 7.92-7.77 |
| | | | (m, 2H), 7.61-7.51 (m, 1H), 7.48-7.40 (m, 1H), 7.32 (br d, J = |
| | | | 7.8 Hz, 1H), 7.06-6.89 (m, 1H), 6.59-6.26 (m, 2H), 5.99 (d, |
| | | | J = 7.8 Hz, 1H), 4.30 (ddd, J = 4.8, 7.1, 11.6 Hz, 2H), 3.93 (br d, |
| | | | J = 10.5 Hz, 2H), 3.65 (br t, J = 5.4 Hz, 2H), 3.21-3.07 (m, 2H), |
| | | | 2.90 (br d, J = 10.0 Hz, 2H), 2.79-2.65 (m, 2H), 2.62-2.53 (m, |
| | | | 3H), 2.42 (br t, J = 6.3 Hz, 2H), 2.17-2.02 (m, 3H), 2.01-1.73 |
| | | | (m, 11H), 1.71-1.59 (m, 5H), 1.54-1.21 (m, 5H), 0.94 (q, J = |
| | | | 12.1 Hz, 2H) |
| 686 | 785 | 786 | δ (DMSO-d6) 10.79 (s, 1H), 8.43-8.39 (m, 1H), 8.15 (s, 1H), |
| | | | 7.61-7.52 (m, 1H), 7.46-7.37 (m, 2H), 7.21 (d, J = 7.5 Hz, 1H), |
| | | | 7.18-7.14 (m, 1H), 6.85 (t, J = 9.4 Hz, 1H), 6.53 (dd, J = 2.3, |
| | | | 15.0 Hz, 1H), 6.44 (dd, J = 2.2, 8.8 Hz, 1H), 5.83 (br d, J = 7.6 |
| | | | Hz, 1H), 4.45-4.37 (m, 1H), 4.32-4.23 (m, 1H), 4.01-3.91 (m, |
| | | | 1H), 3.81-3.76 (m, 4H), 3.71-3.59 (m, 1H), 3.21-3.15 (m, 4H), |
| | | | 3.08-2.96 (m, 2H), 2.89 (br s, 4H), 2.82-2.67 (m, 2H), 2.63- |
| | | | 2.55 (m, 4H), 2.32-2.16 (m, 2H), 2.14-1.98 (m, 3H), 1.90-1.68 |
| | | | (m, 6H), 1.56-1.29 (m, 4H), 1.11-0.84 (m, 2H) |
| 687 | 785 | 786 | δ (DMSO-d6) 10.79 (s, 1H), 8.39 (d, J = 3.6 Hz, 1H), 7.55 (br s, |
| | | | 1H), 7.34 (br d, J = 4.3 Hz, 2H), 7.20-7.07 (m, 2H), 6.82 (t, J = |
| | | | 9.4 Hz, 1H), 6.51 (br d, J = 15.3 Hz, 1H), 6.42 (br d, J = 8.8 Hz, |
| | | | 1H), 5.81 (br d, J = 7.6 Hz, 1H), 4.50 (br s, 1H), 4.32-4.20 (m, |
| | | | 1H), 4.00 (br d, J = 10.8 Hz, 2H), 3.78-3.65 (m, 1H), 3.24-3.15 |
| | | | (m, 4H), 2.84 (br s, 4H), 2.73 (ddd, J = 5.2, 12.1, 17.6 Hz, 3H), |
| | | | 2.60 (br s, 4H), 2.55 (br d, J = 3.9 Hz, 1H), 2.42 (br t, J = 10.1 |
| | | | Hz, 1H), 2.09 (td, J = 4.3, 8.5 Hz, 1H), 1.98 (br d, J = 8.3 Hz, |
| | | | 4H), 1.85 (br dd, J = 4.2, 11.8 Hz, 1H), 1.78 (br d, J = 12.0 Hz, |
| | | | 2H), 1.64 (br d, J = 3.9 Hz, 4H), 1.57 (br d, J = 4.3 Hz, 2H), 1.42 |
| | | | (br s, 4H), 1.36-1.22 (m, 2H) |
| 688 | 801 | 802 | δ (DMSO-d6) 10.79 (br s, 1H), 8.46 (d, J = 3.6 Hz, 1H), 8.01 (s, |
| | | | 1H), 7.93-7.86 (m, 1H), 7.62-7.57 (m, 2H), 7.24 (br d, J = 7.8 |
| | | | Hz, 1H), 6.83 (t, J = 9.3 Hz, 1H), 6.52 (dd, J = 2.0, 15.0 Hz, 1H), |
| | | | 6.45-6.41 (m, 1H), 5.81 (d, J = 7.6 Hz, 1H), 4.50 (br s, 1H), 4.31- |
| | | | 4.24 (m, 3H), 4.04-3.98 (m, 4H), 3.84-3.71 (m, 3H), 2.85 (br |
| | | | s, 5H), 2.81-2.68 (m, 3H), 2.61 (br d, J = 3.6 Hz, 4H), 2.44 (br |
| | | | d, J = 11.9 Hz, 1H), 2.10 (td, J = 4.1, 8.7 Hz, 1H), 2.02-1.95 (m, |
| | | | 4H), 1.92-1.83 (m, 1H), 1.79 (br d, J = 11.8 Hz, 2H), 1.48-1.39 |
| | | | (m, 4H), 1.37-1.27 (m, 2H) |
| 689 | 784 | 785 | δ (DMSO-d6) 10.78 (s, 1H), 8.39 (d, J = 3.8 Hz, 1H), 8.19 (s, |
| | | | 1H), 7.55 (br s, 1H), 7.34 (br d, J = 4.9 Hz, 2H), 7.16-7.08 (m, |
| | | | 2H), 6.98 (t, J = 8.8 Hz, 1H), 6.49-6.41 (m, 2H), 5.99 (d, J = 7.6 |
| | | | Hz, 1H), 4.50 (br s, 1H), 4.30 (br s, 1H), 4.02 (br d, J = 12.3 Hz, |
| | | | 2H), 3.70 (br s, 2H), 3.23-3.17 (m, 5H), 2.94 (br d, J = 10.8 Hz, |
| | | | 2H), 2.80-2.69 (m, 3H), 2.63-2.56 (m, 2H), 2.27 (br t, J = 10.1 |
| | | | Hz, 2H), 2.12-2.06 (m, 1H), 1.98 (br d, J = 8.6 Hz, 4H), 1.91- |
| | | | 1.84 (m, 1H), 1.76 (br d, J = 11.6 Hz, 2H), 1.64 (br s, 6H), 1.59- |
| | | | 1.52 (m, 3H), 1.46-1.39 (m, 4H), 1.37-1.27 (m, 2H) |
| 690 | 800 | 801 | δ (DMSO-d6) 10.81 (s, 1H), 8.47 (d, J = 3.6 Hz, 1H), 8.03-7.99 |
| | | | (m, 1H), 7.89 (br d, J = 5.5 Hz, 1H), 7.64-7.57 (m, 1H), 7.31- |
| | | | 7.14 (m, 1H), 6.97 (br t, J = 8.6 Hz, 1H), 6.56-6.45 (m, 1H), 4.53 |
| | | | (br s, 1H), 4.37-4.31 (m, 1H), 4.25 (s, 2H), 4.13 (br d, J = 11.0 |
| | | | Hz, 2H), 4.05-3.99 (m, 2H), 3.84-3.78 (m, 2H), 3.76 (br d, J = |
| | | | 3.4 Hz, 1H), 3.49 (br d, J = 10.9 Hz, 2H), 3.37 (br d, J = 1.9 Hz, |
| | | | 1H), 3.16-3.04 (m, 2H), 3.03-2.93 (m, 1H), 2.91-2.82 (m, 1H), |
| | | | 2.81-2.67 (m, 2H), 2.61 (br d, J = 3.9 Hz, 1H), 2.57 (br s, 1H), |
| | | | 2.26-2.03 (m, 6H), 2.00 (br d, J = 9.0 Hz, 4H), 1.92-1.79 (m, |
| | | | 3H), 1.69-1.58 (m, 2H), 1.57-1.32 (m, 5H) |
| 691 | 808 | 809 | δ (DMSO-d6) 10.85-10.72 (m, 1H), 8.48 (d, J = 3.6 Hz, 1H), |
| | | | 8.08 (br d, J = 7.4 Hz, 1H), 7.99 (s, 1H), 7.76-7.67 (m, 2H), 7.64- |
| | | | 7.52 (m, 2H), 7.32-7.26 (m, 1H), 6.83 (t, J = 9.4 Hz, 1H), 6.56- |
| | | | 6.47 (m, 2H), 6.44-6.33 (m, 2H), 5.81-5.75 (m, 1H), 4.29- |
| | | | 4.21 (m, 2H), 4.05 (br d, J = 13.1 Hz, 1H), 3.65 (br s, 2H), 3.07 |
| | | | (br d, J = 11.1 Hz, 2H), 2.95 (br t, J = 11.3 Hz, 1H), 2.79-2.68 |
| | | | (m, 1H), 2.62-2.54 (m, 4H), 2.14-2.06 (m, 1H), 2.00 (br d, J = |
| | | | 10.0 Hz, 2H), 1.91-1.77 (m, 3H), 1.71 (br d, J = 12.0 Hz, 2H), |
| | | | 1.59 (br d, J = 11.8 Hz, 2H), 1.55-1.41 (m, 4H), 1.41-1.26 (m, |
| | | | 8H) |
| 692 | 828 | 829 | δ 10.80 (s, 1H), 8.45 (d, J = 3.5 Hz, 1H), 8.16 (s, 1H), 8.06 (br d, |
| | | | J = 7.5 Hz, 1H), 7.97 (s, 1H), 7.75-7.67 (m, 2H), 7.63-7.47 (m, |
| | | | 3H), 7.21 (br d, J = 7.8 Hz, 1H), 7.07 (br t, J = 8.3 Hz, 1H), 6.60- |
| | | | 6.43 (m, 3H), 6.35 (dt, J = 1.2, 6.7 Hz, 1H), 6.15 (br d, J = 7.8 |
| | | | Hz, 1H), 4.46-4.25 (m, 1H), 3.64 (br d, J = 5.3 Hz, 1H), 3.51- |
| | | | 3.39 (m, 1H), 3.21-3.04 (m, 2H), 2.92 (br d, J = 9.8 Hz, 1H), |
| | | | 2.81-2.66 (m, 1H), 2.64-2.52 (m, 2H), 2.44-2.31 (m, 2H), 2.16- |
| | | | 2.02 (m, 2H), 1.98 (br d, J = 12.1 Hz, 3H), 1.88 (br dd, J = 4.3, |
| | | | 12.3 Hz, 1H), 1.81 (br s, 7H), 1.53-1.38 (m, 2H), 1.36-1.22 (m, |
| | | | 4H), 1.21-1.08 (m, 2H) |
| 693 | 792 | 793 | δ 10.79 (s, 1H), 8.48 (d, J = 3.5 Hz, 1H), 8.13 (s, 1H), 8.05 (br d, |
| | | | J = 7.4 Hz, 1H), 7.98 (s, 1H), 7.75-7.66 (m, 2H), 7.60 (dd, J = |
| | | | 0.9, 7.9 Hz, 1H), 7.54 (ddd, J = 2.0, 6.8, 9.1 Hz, 1H), 7.35 (br d, |
| | | | J = 7.6 Hz, 1H), 6.97 (br t, J = 8.5 Hz, 1H), 6.55-6.41 (m, 3H), |
| | | | 6.35 (dt, J = 1.1, 6.7 Hz, 1H), 6.04 (br d, J = 7.9 Hz, 1H), 4.39- |
| | | | 4.21 (m, 2H), 3.92 (br d, J = 12.3 Hz, 2H), 3.22-3.08 (m, 3H), |
| | | | 2.80-2.53 (m, 6H), 2.16-1.52 (m, 15H), 1.50-1.18 (m, 5H), |
| | | | 1.00 (br d, J = 11.3 Hz, 2H) |
| 694 | 810 | 811 | δ 10.78 (s, 1H), 8.46 (d, J = 3.6 Hz, 1H), 8.04 (d, J = 7.9 Hz, 1H), |
| | | | 7.97 (s, 1H), 7.76-7.66 (m, 3H), 7.62-7.50 (m, 2H), 7.21 (br d, |
| | | | J = 8.0 Hz, 1H), 7.01-6.94 (m, 1H), 6.51 (d, J = 9.0 Hz, 1H), |
| | | | 6.46-6.40 (m, 2H), 6.35 (dt, J = 1.2, 6.7 Hz, 1H), 5.98 (d, J = 7.8 |
| | | | Hz, 1H), 5.21-4.99 (m, 1H), 4.34-4.22 (m, 1H), 3.63 (br dd, |
| | | | J = 2.9, 5.6 Hz, 1H), 3.53-3.43 (m, 1H), 2.98 (br d, J = 8.0 Hz, |
| | | | 2H), 2.78-2.64 (m, 2H), 2.42-2.31 (m, 4H), 2.13-2.03 (m, 1H), |
| | | | 1.98-1.82 (m, 4H), 1.81-1.72 (m, 3H), 1.70-1.53 (m, 7H), 1.50- |
| | | | 1.38 (m, 1H), 1.36-1.19 (m, 5H). |
| 695 | 812 | 813 | δ 10.78 (br s, 1H), 8.47 (d, J = 3.8 Hz, 1H), 8.06 (br d, J = 7.5 Hz, |
| | | | 1H), 7.98 (s, 1H), 7.77-7.66 (m, 2H), 7.63-7.50 (m, 2H), 7.25 |
| | | | (d, J = 7.8 Hz, 1H), 7.07-6.91 (m, 1H), 6.51 (d, J = 9.0 Hz, 1H), |
| | | | 6.47-6.38 (m, 2H), 6.35 (dt, J = 1.2, 6.7 Hz, 1H), 5.98 (d, J = 7.6 |
| | | | Hz, 1H), 5.11-4.87 (m, 1H), 4.58-4.43 (m, 1H), 4.37-3.98 (m, |
| | | | 3H), 3.73 (br d, J = 5.8 Hz, 1H), 3.12-2.93 (m, 3H), 2.92-2.77 |
| | | | (m, 1H), 2.75-2.62 (m, 1H), 2.62-2.54 (m, 2H), 2.38-2.28 (m, |
| | | | 2H), 2.18-2.04 (m, 1H), 2.02-1.86 (m, 5H), 1.84-1.70 (m, 1H), |
| | | | 1.68-1.55 (m, 5H), 1.49-1.17 (m, 5H) |
| 696 | 793 | 794 | δ 10.80 (s, 1H), 9.08 (br s, 1H), 8.80 (d, J = 5.3 Hz, 1H), 8.56 (d, |
| | | | J = 3.3 Hz, 1H), 8.30 (s, 1H), 8.08-7.91 (m, 2H), 7.75-7.64 (m, |
| | | | 1H), 7.57 (ddd, J = 2.0, 6.7, 9.1 Hz, 1H), 7.38 (br d, J = 7.6 Hz, |
| | | | 1H), 6.95 (br t, J = 8.9 Hz, 1H), 6.59-6.37 (m, 4H), 4.32 (br dd, |
| | | | J = 4.8, 11.5 Hz, 1H), 3.49 (br d, J = 11.1 Hz, 3H), 3.24-3.08 |
| | | | (m, 3H), 3.00-2.89 (m, 1H), 2.74 (ddd, J = 5.3, 12.2, 17.6 Hz, |
| | | | 1H), 2.62-2.55 (m, 1H), 2.19-1.96 (m, 7H), 1.91 (br d, J = 10.5 |
| | | | Hz, 6H), 1.74 (br d, J = 11.1 Hz, 2H), 1.68-1.36 (m, 5H), 1.35- |
| | | | 1.15 (m, 4H) |
| 697 | 822 | 823 | δ (DMSO-d6) 10.79 (s, 1H), 8.47 (d, J = 3.5 Hz, 1H), 8.20 (s, |
| | | | 1H), 8.06 (br d, J = 7.1 Hz, 1H), 7.98 (s, 1H), 7.74-7.67 (m, 2H), |
| | | | 7.62-7.53 (m, 2H), 7.25 (d, J = 7.5 Hz, 1H), 6.99 (t, J = 8.9 Hz, |
| | | | 1H), 6.52 (d, J = 9.3 Hz, 1H), 6.47-6.41 (m, 2H), 6.36 (t, J = 6.7 |
| | | | Hz, 1H), 6.00 (d, J = 7.8 Hz, 1H), 4.52-4.46 (m, 1H), 4.35-4.24 |
| | | | (m, 1H), 3.95 (br d, J = 12.8 Hz, 2H), 3.72 (br d, J = 6.5 Hz, 1H), |
| | | | 2.96 (br d, J = 10.9 Hz, 2H), 2.73 (dt, J = 11.9, 6.0 Hz, 3H), 2.59 |
| | | | (br s, 1H), 2.34 (br t, J = 7.3 Hz, 2H), 2.12-2.05 (m, 1H), 2.00- |
| | | | 1.93 (m, 6H), 1.86 (br dd, J = 12.9, 4.4 Hz, 1H), 1.68-1.60 (m, |
| | | | 6H), 1.50-1.34 (m, 8H), 1.05-0.95 (m, 2H) |
| 698 | 795 | 796 | δ (DMSO-d6) 10.79 (s, 1H), 8.49 (d, J = 3.5 Hz, 1H), 8.07 (br d, |
| | | | J = 7.6 Hz, 1H), 7.99 (s, 1H), 7.77-7.68 (m, 2H), 7.61 (br d, J = |
| | | | 7.9 Hz, 1H), 7.56 (ddd, J = 1.9, 6.8, 9.0 Hz, 1H), 7.28 (d, J = 7.6 |
| | | | Hz, 1H), 6.84 (t, J = 9.4 Hz, 1H), 6.56-6.48 (m, 2H), 6.42 (br d, |
| | | | J = 8.6 Hz, 1H), 6.37 (t, J = 6.7 Hz, 1H), 5.81 (d, J = 7.5 Hz, 1H), |
| | | | 4.51 (br s, 1H), 4.41-4.21 (m, 1H), 3.81-3.67 (m, 1H), 3.40- |
| | | | 3.35 (m, 4H), 3.17 (br d, J = 10.8 Hz, 2H), 2.81-2.69 (m, 1H), |
| | | | 2.62-2.54 (m, 3H), 2.49 (br s, 3H), 2.34 (br d, J = 4.1 Hz, 1H), |
| | | | 2.15-2.04 (m, 1H), 1.98 (br d, J = 9.3 Hz, 4H), 1.92-1.77 (m, |
| | | | 3H), 1.67-1.30 (m, 7H) |
| 699 | 795 | 796 | δ 10.79 (s, 1H), 8.50 (d, J = 3.6 Hz, 1H), 8.35 (s, 0.3H), 8.07 (d, |
| | | | J = 7.6 Hz, 1H), 7.99 (s, 1H), 7.76-7.68 (m, 2H), 7.64-7.59 (m, |
| | | | 1H), 7.56 (ddd, J = 9.0, 6.6, 2.0 Hz, 1H), 7.36 (d, J = 7.8 Hz, 1H), |
| | | | 6.83 (t, J = 9.6 Hz, 1H), 6.58-6.47 (m, 2H), 6.46-6.40 (m, 1H), |
| | | | 6.37 (dt, J = 6.8, 1.2 Hz, 1H), 5.81 (d, J = 7.6 Hz, 1H), 4.53-4.39 |
| | | | (m, 1H), 4.33-4.20 (m, 1H), 3.98-3.90 (m, 3H), 3.03-2.90 (m, |
| | | | 3H), 2.85 (br s, 4H), 2.79-2.71 (m, 1H), 2.61 (br s, 4H), 2.34- |
| | | | 2.25 (m, 1H), 2.16-2.05 (m, 1H), 2.02-1.95 (m, 2H), 1.93-1.82 |
| | | | (m, 5H), 1.43-1.34 (m, 6H) |
| 700 | 801 | 802 | δ (DMSO-d6) 10.83-10.74 (m, 1H), 8.38 (d, J = 3.9 Hz, 1H), |
| | | | 7.65-7.48 (m, 2H), 7.35 (br d, J = 4.4 Hz, 2H), 7.12 (br d, J = |
| | | | 7.4 Hz, 2H), 6.82 (t, J = 9.3 Hz, 1H), 6.51 (dd, J = 15.2, 2.3 Hz, |
| | | | 1H), 6.44-6.39 (m, 1H), 5.82 (d, J = 7.3 Hz, 1H), 5.10-4.88 (m, |
| | | | 1H), 4.32-4.22 (m, 1H), 3.68-3.59 (m, 1H), 3.56-3.44 (m, 1H), |
| | | | 3.20 (br t, J = 5.1 Hz, 5H), 2.93 (br d, J = 7.4 Hz, 2H), 2.84-2.62 |
| | | | (m, 4H), 2.41-2.25 (m, 3H), 2.13-1.98 (m, 5H), 1.95 (br s, 1H), |
| | | | 1.90-1.81 (m, 1H), 1.78-1.69 (m, 4H), 1.64 (br d, J = 3.9 Hz, |
| | | | 5H), 1.60-1.55 (m, 2H), 1.51-1.38 (m, 3H), 1.37-1.24 (m, 4H) |
| 701 | 815 | 816 | δ (DMSO-d6) 10.78 (s, 1H), 8.43 (d, J = 3.6 Hz, 1H), 7.87 (s, |
| | | | 1H), 7.85 (br d, J = 7.9 Hz, 1H), 7.61 (br d, J = 7.9 Hz, 1H), 7.56 |
| | | | (t, J = 7.9 Hz, 1H), 7.45 (br d, J = 8.4 Hz, 1H), 7.18 (br d, J = 7.9 |
| | | | Hz, 1H), 6.82 (t, J = 9.4 Hz, 1H), 6.51 (dd, J = 15.1, 2.3 Hz, 1H), |
| | | | 6.42 (dd, J = 8.7, 1.9 Hz, 1H), 5.82 (d, J = 7.5 Hz, 1H), 5.06- |
| | | | 4.90 (m, 1H), 4.30-4.22 (m, 1H), 3.65 (br t, J = 5.4 Hz, 3H), 3.54- |
| | | | 3.45 (m, 1H), 3.21 (br d, J = 9.6 Hz, 1H), 2.93 (br d, J = 7.6 Hz, |
| | | | 2H), 2.82 (br d, J = 12.4 Hz, 1H), 2.77-2.62 (m, 3H), 2.59 (br t, |
| | | | J = 3.9 Hz, 1H), 2.42 (br t, J = 6.3 Hz, 2H), 2.35-2.26 (m, 2H), |
| | | | 2.12-2.04 (m, 2H), 2.02-1.97 (m, 2H), 1.91-1.84 (m, 5H), 1.77- |
| | | | 1.67 (m, 5H), 1.51-1.43 (m, 2H), 1.42-1.34 (m, 2H), 1.33- |
| | | | 1.25 (m, 3H), 1.09-0.99 (m, 1H) |
| 702 | 793 | 794 | δ 11.90 (br s, 1H), 10.82 (s, 1H), 8.68-8.13 (m, 2H), 7.98-7.85 |
| | | | (m, 2H), 7.75 (dd, J = 2.0, 6.9 Hz, 1H), 7.67 (br d, J = 3.5 Hz, |
| | | | 1H), 7.59 (br t, J = 7.8 Hz, 1H), 7.51-7.42 (m, 1H), 7.20 (br d, |
| | | | J = 7.8 Hz, 1H), 6.87 (br t, J = 9.4 Hz, 1H), 6.54 (br d, J = 15.0 Hz, |
| | | | 1H), 6.49-6.42 (m, 1H), 6.38 (t, J = 6.7 Hz, 1H), 5.84 (br d, J = |
| | | | 7.5 Hz, 1H), 4.35-4.23 (m, 1H), 3.75-3.66 (m, 1H), 3.59-3.52 |
| | | | (m, 1H), 3.21 (br d, J = 9.9 Hz, 2H), 3.01 (br d, J = 6.8 Hz, 2H), |
| | | | 2.75 (br dd, J = 5.2, 12.1 Hz, 1H), 2.66-2.59 (m, 2H), 2.36-2.19 |
| | | | (m, 2H), 2.12 (br dd, J = 4.3, 8.5 Hz, 2H), 2.01 (br d, J = 7.3 Hz, |
| | | | 2H), 1.94-1.78 (m, 5H), 1.76-1.53 (m, 6H), 1.48-1.20 (m, 6H) |
| 703 | 821 | 822 | δ (DMSO-d6) 11.94-11.75 (m, 1H), 10.82-10.73 (m, 1H), 8.42 |
| | | | (d, J = 3.6 Hz, 1H), 8.32 (s, 1H), 7.93-7.85 (m, 2H), 7.72-7.62 |
| | | | (m, 2H), 7.60-7.52 (m, 1H), 7.44 (dd, J = 6.3, 1.6 Hz, 1H), 7.16 |
| | | | (br d, J = 7.8 Hz, 1H), 6.82 (t, J = 9.3 Hz, 1H), 6.51 (dd, J = 15.1, |
| | | | 2.3 Hz, 1H), 6.42 (dd, J = 8.6, 2.3 Hz, 1H), 6.33 (t, J = 6.7 Hz, |
| | | | 1H), 5.80 (d, J = 7.6 Hz, 1H), 4.31-4.20 (m, 1H), 3.72-3.61 (m, |
| | | | 1H), 3.55-3.45 (m, 1H), 2.84 (br s, 3H), 2.73 (td, J = 6.1, 17.5 |
| | | | Hz, 1H), 2.59 (br d, J = 4.4 Hz, 1H), 2.56-2.53 (m, 1H), 2.44 (br |
| | | | s, 4H), 2.14-2.06 (m, 3H), 2.01-1.95 (m, 2H), 1.93-1.88 (m, |
| | | | 2H), 1.85-1.74 (m, 5H), 1.67 (br d, J = 11.8 Hz, 2H), 1.62-1.54 |
| | | | (m, 1H), 1.48-1.39 (m, 1H), 1.36-1.23 (m, 4H), 0.98-0.79 (m, |
| | | | 4H) |
| 704 | 821 | 822 | δ 10.77 (s, 1H), 8.46 (d, J = 3.6 Hz, 1H), 8.15 (s, 1H), 8.04 (br d, |
| | | | J = 7.6 Hz, 1H), 7.97 (br s, 1H), 7.75-7.66 (m, 3H), 7.62-7.51 |
| | | | (m, 2H), 7.22 (br d, J = 7.8 Hz, 1H), 6.82 (t, J = 9.3 Hz, 1H), 6.54- |
| | | | 6.46 (m, 2H), 6.43-6.32 (m, 2H), 5.78 (br d, J = 7.6 Hz, 1H), |
| | | | 4.30-4.19 (m, 1H), 3.64 (br dd, J = 1.4, 3.8 Hz, 1H), 3.51 (br dd, |
| | | | J = 3.5, 7.4 Hz, 1H), 3.10 (br d, J = 11.3 Hz, 2H), 2.85 (br d, J = |
| | | | 10.4 Hz, 2H), 2.78-2.63 (m, 2H), 2.62-2.54 (m, 2H), 2.22 (br |
| | | | d, J = 6.1 Hz, 2H), 2.13-2.04 (m, 1H), 2.03-1.88 (m, 6H), 1.86- |
| | | | 1.70 (m, 5H), 1.68-1.52 (m, 4H), 1.37-1.11 (m, 8H) |
| 705 | 832 | 833 | δ (DMSO-d6) 11.06-10.99 (m, 1H), 8.52-8.44 (m, 1H), 7.99 |
| | | | (br s, 1H), 7.84-7.70 (m, 6H), 7.65-7.58 (m, 1H), 7.48 (s, 1H), |
| | | | 7.43-7.32 (m, 3H), 5.13 (dd, J = 13.3, 5.1 Hz, 1H), 4.51-4.42 |
| | | | (m, 1H), 4.38-4.27 (m, 2H), 3.84 (br s, 11H), 3.63-3.49 (m, |
| | | | 4H), 3.24 (br d, J = 10.8 Hz, 3H), 3.05-2.90 (m, 4H), 2.62 (br d, |
| | | | J = 16.6 Hz, 1H), 2.41 (br dd, J = 13.2, 4.5 Hz, 1H), 2.13-2.06 |
| | | | (m, 2H), 2.02 (br d, J = 5.3 Hz, 2H), 1.88-1.72 (m, 2H), 1.54 (br |
| | | | d, J = 10.8 Hz, 1H), 1.47-1.39 (m, 1H) |
| 706 | 792 | 793 | δ (DMSO-d6) 10.79 (s, 1H), 8.47 (d, J = 3.6 Hz, 1H), 8.05 (br d, |
| | | | J = 8.0 Hz, 1H), 7.98 (s, 1H), 7.75-7.68 (m, 2H), 7.62-7.59 (m, |
| | | | 1H), 7.58-7.48 (m, 2H), 7.23 (br d, J = 8.6 Hz, 1H), 7.03-6.98 |
| | | | (m, 1H), 6.55-6.50 (m, 1H), 6.47-6.41 (m, 2H), 6.39-6.33 (m, |
| | | | 1H), 6.02-5.96 (m, 1H), 4.35-4.27 (m, 1H), 3.70-3.58 (m, 1H), |
| | | | 3.57-3.40 (m, 2H), 3.04-2.97 (m, 2H), 2.60 (br d, J = 3.5 Hz, |
| | | | 1H), 2.33-2.15 (m, 3H), 2.13-2.05 (m, 2H), 2.05-1.93 (m, 4H), |
| | | | 1.89-1.84 (m, 2H), 1.83-1.75 (m, 4H), 1.68-1.63 (m, 2H), 1.62- |
| | | | 1.53 (m, 2H), 1.48-1.41 (m, 2H), 1.40-1.35 (m, 2H), 1.34 (br |
| | | | d, J = 1.2 Hz, 3H), 1.26-1.23 (m, 1H) |

### Biological Examples

### Example B-1: Enzyme Binding Assays (KINOMEscan)

PIP4K2C binding constants disclosed herein were determined using the proprietary Human PIP4K2C binding assay (Catalog number 87-0007-1094, Eurofins DiscoverX, San Diego, CA). This assay measures active site-directed competition binding to an immobilized ligand (see https://doi.org/10.1038/nbt1068, herein incorporated by reference in its entirety). Experiments were conducted using an 11-point dose response curve, in duplicate, with a top starting concentration of 5 micromolar; the Levenberg-Marquardt algorithm was used to fit a Hill equation to dose-response data to calculate IC50 values (see Table 5).

TABLE 5

| | Pip4k2c | | Pip4k2c |
|---|---|---|---|
| | Kd: | | Kd: |
| Cmpd | IC50 | Cmpd | IC50 |
| No. | (nM) | No. | (nM) |
| 1 | B | 23 | A |
| 2 | A | 24 | C |
| 3 | A | 25 | B |
| 4 | A | 26 | B |
| 5 | A | 27 | B |
| 6 | A | 28 | C |
| 7 | A | 29 | C |
| 8 | A | 30 | A |
| 9 | A | 31 | B |
| 10 | A | 32 | A |
| 11 | A | 33 | A |
| 12 | A | 34 | A |
| 13 | A | 35 | A |
| 14 | A | 36 | A |
| 15 | A | 37 | A |
| 16 | A | 38 | A |
| 17 | A | 39 | A |
| 18 | A | 40 | A |
| 19 | A | 41 | A |
| 20 | C | 42 | C |
| 21 | A | 43 | B |
| 22 | A | 91 | A |
| 92 | A | 197 | C |
| 93 | A | 198 | C |
| 94 | A | 199 | B |
| 95 | C | 200 | A |
| 96 | B | 201 | A |
| 97 | A | 202 | A |
| 98 | A | 203 | A |
| 99 | B | 204 | A |
| 100 | A | 205 | B |
| 101 | A | 206 | A |
| 102 | A | 207 | A |
| 103 | A | 208 | A |
| 104 | A | 209 | A |
| 105 | A | 210 | A |
| 106 | B | 211 | A |
| 107 | B | 212 | A |
| 108 | C | 213 | A |
| 109 | C | 214 | A |
| 110 | A | 215 | C |
| 111 | A | 216 | B |
| 112 | A | 217 | A |
| 113 | C | 218 | A |
| 114 | A | 219 | A |
| 115 | A | 220 | A |
| 116 | A | 221 | A |
| 117 | A | 222 | A |
| 118 | A | 223 | A |
| 119 | A | 224 | B |
| 120 | A | 225 | A |
| 121 | A | 226 | A |
| 122 | A | 227 | A |
| 123 | B | 228 | A |
| 124 | A | 229 | A |
| 125 | C | 230 | A |
| 126 | C | 231 | A |
| 127 | B | 232 | A |
| 128 | A | 233 | A |
| 129 | A | 234 | A |
| 130 | A | 235 | A |
| 131 | A | 236 | A |
| 132 | A | 237 | A |
| 133 | B | 238 | A |
| 134 | A | 239 | A |
| 135 | A | 240 | A |
| 136 | A | 241 | A |
| 137 | A | 242 | A |
| 138 | A | 243 | A |
| 139 | A | 244 | A |
| 140 | A | 245 | A |
| 141 | A | 246 | A |
| 142 | A | 247 | A |
| 143 | B | 248 | A |
| 144 | C | 249 | A |
| 145 | A | 250 | A |
| 146 | A | 251 | A |
| 147 | A | 252 | A |
| 148 | C | 253 | A |
| 149 | A | 254 | A |
| 150 | A | 255 | A |
| 152 | A | 256 | A |
| 153 | A | 257 | A |
| 154 | A | 258 | A |
| 155 | A | 259 | A |
| 156 | A | 260 | C |
| 157 | A | 261 | A |
| 158 | A | 262 | A |
| 159 | A | 263 | A |
| 160 | A | 264 | A |
| 161 | A | 265 | A |
| 162 | A | 266 | A |
| 163 | B | 267 | A |
| 164 | A | 268 | A |
| 165 | B | 269 | A |
| 166 | A | 270 | A |
| 167 | B | 271 | A |
| 168 | A | 272 | A |
| 169 | A | 273 | A |
| 170 | A | 274 | A |
| 171 | A | 606 | A |
| 172 | A | 607 | A |
| 173 | A | 608 | A |
| 174 | A | 609 | A |
| 175 | A | 610 | A |
| 176 | A | 611 | A |
| 177 | A | 612 | A |
| 178 | A | 613 | A |
| 179 | A | 614 | A |
| 180 | A | 615 | A |
| 181 | A | 616 | A |
| 182 | C | 617 | A |
| | | 618 | A |
| | | 619 | A |
| | | 620 | A |
| | | 621 | A |
| 187 | A | 622 | A |
| 188 | A | 623 | A |
| 189 | C | 624 | A |
| 190 | A | 625 | A |
| 191 | A | 626 | A |
| 192 | A | 627 | A |
| 193 | A | 628 | A |
| 194 | C | 629 | A |
| 195 | A | 630 | A |
| 196 | A | 631 | A |

Legend:
A = <500 nM,
B = 500 nM-1 μM and
C = 1 μM-5 μM

### Example B-2: HiBiT PIP4K2C degradation assay

Materials: MOLT-4, a human T lymphoblast cell line (see https://www.atcc.org/products/crl-1582, herein incorporated by reference in its entirety), was genetically engineered using CRISPR-Cas9 by Synthego Corporation (Redwood City, CA) to C-terminally tag endogenous PIP4K2C with HiBiT peptide. Following clonal selection, genotype was confirmed by Sanger sequencing. MO LT-4 were maintained using RPMI-1640 medium from Gibco (Grand Island, NY, catalog number 11875-085) supplemented with 10% fetal bovine serum (FBS) from VWR (Philadelphia, PA, catalog number 97068-085). For HiBiT assays the cells were resuspended in RPMI-1640 medium without phenol read from Gibco (Grand Island, NY, catalog number 11835-030) supplemented with 10% FBS. Nano-Glo® HiBiT Lytic Assay System was purchased from Promega (Madison, WI, Catalog number N3030). Cell culture flasks and 384-well microplates were purchased from Corning (Glendale, AZ).

To study PIP4K2C degradation, 384-well plates were plated with a 10-point dose response curve, 3-fold dilution, in duplicate with a top starting concentration of 10 micromolar. Subsequently 22,500 cells were added to each well and incubated at 37 C in 5% C02 for 24 hours or 6 hours. At the end of the incubation time, cells were treated with the Nano-Glo® HiBiT Lytic Detection System following the manufacturer's instructions. Luminescence was measured with a PHERAstar FSX plate reader (BMG Labtech, Cary, NC). The Levenberg-Marquardt algorithm was used to fit a Hill equation to dose-response data to calculate DC50 values (see Table 6).

TABLE 6

| | HiBIT-degradation | | HiBIT-degradation |
|---|---|---|---|
| | MOLT4 PIP4k2C | | MOLT4 PIP4k2C |
| Cmpd | 24 hours DC50 | Cmpd | 24 hours DC50 |
| No. | (nM) | No. | (nM) |
| 44 | A | 68 | A |
| 45 | A | 69 | A |
| 46 | A | 70 | A |
| 47 | A | 71 | A |
| 48 | A | 72 | A |
| 49 | A | 73 | A |
| 50 | A | 74 | A |
| 51 | A | 75 | A |
| 52 | A | 76 | A |
| 53 | B | 77 | A |
| 54 | A | 78 | A |
| 55 | B | 79 | A |
| 56 | A | 80 | A |
| 57 | A | 81 | A |
| 58 | A | 82 | A |
| 59 | A | 83 | A |
| 60 | A | 84 | A |
| 61 | A | 85 | A |
| 62 | A | 86 | B |
| 63 | A | 87 | A |
| 64 | A | 88 | A |
| 65 | A | 89 | A |
| 66 | B | 90 | A |
| 67 | A | | |
| 275 | A | 478 | A* |
| 276 | A | 479 | A* |
| 277 | B | 480 | A* |
| 278 | A | 481 | A* |
| 279 | A | 482 | A* |
| 280 | A | 483 | A* |
| 281 | A | 484 | A* |
| 282 | A | 485 | A* |
| 283 | C | 486 | A* |
| 284 | A | 487 | A* |
| 285 | A | 488 | A* |
| 286 | A | 489 | A* |
| 287 | A | 490 | A* |
| 288 | A | 491 | A* |
| 289 | A | 492 | A* |
| 290 | A | 493 | A* |
| 291 | A | 494 | A* |
| 292 | D | 495 | A* |
| 293 | A | 496 | A* |
| 294 | A | 497 | A* |
| 295 | A | 498 | A* |
| 296 | A | 499 | A* |
| 297 | A | 500 | A* |
| 298 | A | 501 | A* |
| 299 | A | 502 | A* |
| 300 | A | 503 | A* |
| 301 | A | 504 | A* |
| 302 | A | 505 | A* |
| 303 | A | 506 | A* |
| 304 | A | 507 | A* |
| 305 | A | 508 | A* |
| 306 | A | 509 | A* |
| 307 | C | 510 | A* |
| 308 | A* | 511 | A* |
| 309 | D | 512 | A* |
| 310 | A | 513 | A* |
| 311 | A | 514 | A* |
| 312 | A | 515 | A* |
| 313 | A | 516 | A* |
| 314 | A | 517 | A* |
| 315 | A | 518 | A* |
| 316 | A | 519 | A* |
| 317 | A | 520 | A* |
| 318 | A | 521 | A* |
| 319 | A | 522 | A* |
| 320 | A | 523 | A* |
| 321 | A | 524 | A* |
| 322 | A | 525 | A* |
| 323 | A | 526 | A* |
| 324 | A | 527 | A* |
| 325 | A | 528 | A* |
| 326 | A | 529 | A* |
| 327 | A | 530 | A* |
| 328 | A | 531 | A* |
| 329 | C | 532 | A* |
| 330 | A | 533 | A* |
| 331 | A | 534 | A* |
| 332 | A | 535 | A* |
| 333 | A | 536 | A* |
| 334 | A | 537 | A* |
| 335 | A | 538 | D* |
| 336 | A | 539 | D* |
| 337 | A | 540 | D* |
| 338 | A | 541 | A* |
| 339 | A | 542 | A* |
| 340 | A | 543 | A* |
| 341 | A | 544 | A* |
| 342 | A | 545 | A* |
| 343 | A | 546 | A* |
| 344 | A | 547 | A* |
| 345 | A* | 548 | A* |
| 346 | D | 549 | A* |
| 347 | A | 550 | A* |
| 348 | A | 551 | A* |
| 349 | A | 552 | A* |
| 350 | A* | 553 | A* |
| 351 | A | 554 | A* |
| 352 | A | 555 | A* |
| 353 | A | 556 | A* |
| 354 | A | 557 | A* |
| 355 | B | 558 | A* |
| 356 | A | 559 | A* |
| 357 | A | 560 | A* |
| 358 | A | 561 | A* |
| 359 | A | 562 | A* |
| 360 | A | 563 | A* |
| 361 | A | 564 | A* |
| 362 | A | 565 | A* |
| 363 | A | 566 | A* |
| 364 | A | 567 | A* |
| 365 | A | 568 | A* |
| 366 | A | 569 | A* |
| 367 | A | 570 | A* |
| 368 | A | 571 | A* |
| 369 | A | 572 | A* |
| 370 | A | 573 | A* |
| 371 | A | 574 | A* |
| 372 | A | 575 | A* |
| 373 | D | 576 | A* |
| 374 | A | 577 | D* |
| 375 | A | 578 | A* |
| 376 | A | 579 | A* |
| 377 | A | 580 | A* |
| 378 | A | 581 | A* |
| 379 | A | 582 | A* |
| 380 | A | 583 | A* |
| 381 | A* | 584 | D* |
| 382 | A* | 585 | A* |
| 383 | A* | 586 | A* |
| 384 | A* | 587 | A* |
| 385 | A* | 588 | A* |
| 386 | A* | 589 | A* |
| 387 | A* | 590 | A* |
| 388 | A* | 591 | A* |
| 389 | A* | 592 | A* |
| 390 | A* | 593 | A* |
| 391 | A* | 594 | A* |
| 392 | A* | 595 | A* |
| 393 | A* | 596 | A* |
| 394 | A* | 597 | A* |
| 395 | A* | 598 | A* |
| 396 | A* | 599 | A* |
| 397 | A* | 600 | A* |
| 398 | A* | 601 | A* |
| 399 | A* | 602 | A* |
| 400 | A* | 603 | A* |
| 401 | A* | 604 | A* |
| 402 | A* | 605 | A* |
| 403 | A* | 632 | A* |
| 404 | D* | 633 | A* |
| 405 | A* | 634 | A* |
| 406 | A* | 635 | A* |
| 407 | A* | 636 | A* |
| 408 | A* | 637 | A* |
| 409 | A* | 638 | A* |
| 410 | A* | 639 | A* |
| 411 | D* | 640 | A* |
| 412 | A* | 641 | A* |
| 413 | A* | 642 | A* |
| 414 | A* | 643 | A* |
| 415 | A* | 644 | A* |
| 416 | A* | 645 | A* |
| 417 | A* | 646 | A* |
| 418 | A* | 647 | A* |
| 419 | A* | 648 | D* |
| 420 | A* | 649 | A* |
| 421 | A* | 650 | D* |
| 422 | A* | 651 | A* |
| 423 | A* | 652 | A* |
| 424 | A* | 653 | A* |
| 425 | A* | 654 | A* |
| 426 | A* | 655 | A* |
| 427 | A* | 656 | A* |
| 428 | A* | 657 | A* |
| 429 | A* | 658 | A* |
| 430 | A* | 659 | A* |
| 431 | A* | 660 | A* |
| 432 | A* | 661 | A* |
| 433 | A* | 662 | A* |
| 434 | A* | 663 | A* |
| 435 | A* | 664 | A* |
| 436 | A* | 665 | A* |
| 437 | A* | 666 | A* |
| 438 | A* | 667 | A* |
| 439 | A* | 668 | D* |
| 440 | A* | 669 | A* |
| 441 | A* | 670 | D* |
| 442 | A* | 671 | A* |
| 443 | A* | 672 | A* |
| 444 | A* | 673 | A* |
| 445 | A* | 674 | A* |
| 446 | A* | 675 | A* |
| 447 | A* | 676 | A* |
| 448 | A* | 677 | A* |
| 449 | A* | 678 | A* |
| 450 | A* | 679 | A* |
| 451 | A* | 680 | A* |
| 452 | A* | 681 | A* |
| 453 | A* | 682 | A* |
| 454 | A* | 683 | D* |
| 455 | A* | 684 | A* |
| 456 | A* | 685 | A* |
| 457 | A* | 686 | A* |
| 458 | A* | 687 | A* |
| 459 | A* | 688 | A* |
| 460 | A* | 689 | A* |
| 461 | A* | 690 | A* |
| 462 | A* | 691 | A* |
| 463 | A* | 692 | A* |
| 464 | A* | 693 | A* |
| 465 | A* | 694 | A* |
| 466 | A* | 695 | A* |
| 467 | A* | 696 | A* |
| 468 | A* | 697 | A* |
| 469 | A* | 698 | A* |
| 470 | A* | 699 | A* |
| 471 | A* | 700 | A* |
| 472 | A* | 701 | A* |
| 473 | A* | 702 | A* |
| 474 | A* | 703 | A* |
| 475 | A* | 704 | A* |
| 476 | A* | 705 | A |
| 477 | A* | 706 | A* |

*6 hours of incubation time, rather than 24 hours
Legend:
A = <500 nM,
B = 500 nM-1 μM,
C = 1 μM-5 μM,
and D = >5 μM

### Example B-3: Degradation in Primary Human PBMC

The ability of certain compounds of Table 1 described by Formula (IV-a) to degrade endogenous PIP4K2C in primary human cells was evaluated in vitro in primary human PBMCs. Briefly, cryopreserved human PBMCs were thawed and incubated in the presence of a range of concentrations of a given compound or DMSO for 6 hours. Cells were fixed and permeabilized, labelled intracellularly with an AlexaFluor647-conjugated monoclonal anti-PIP4K2C antibody, and fluorescence was measured by flow-cytometry. Degradation was measured as percentage of PIP4K2C positive cells compared to DMSO (see Table 7). The Levenberg-Marquardt algorithm was used to fit a Hill equation to dose-response data to calculate DC50 values.

TABLE 7
Degradation DC50 for indicated compound
in primary human PBMCs at 6 h

| | DC50 |
|---|---|
| Compound | (nM) |
| P | 0.300 |
| Q | 0.987 |
| R | 1.000 |
| S | 1.127 |
| T | 1.161 |
| U | 2.999 |
| V | 4.655 |
| W | 6.098 |
| Y | 30.80 |

### Example B-4: Modulation of Phagocytosis

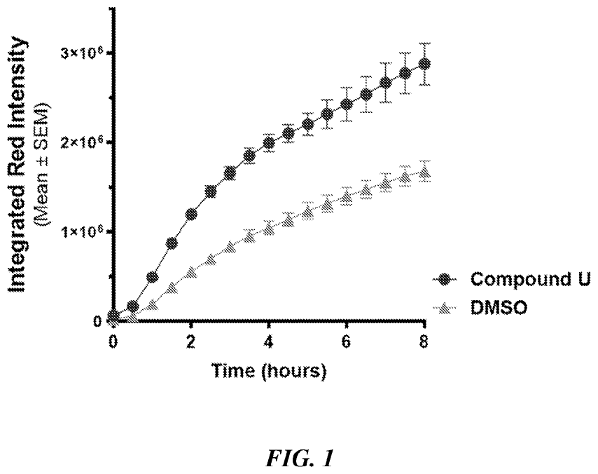

Primary human monocytes were isolated from PBMCs by negative bead selection and differentiated in vitro into immature dendritic cells in the presence of rhGM-CSF (800 u/ml) and rhIL-4 (400 U/mL) for 6 days, in the presence of Compound U (50 nM) or DMSO. The ability of dendritic cells to engulf pHrodo red bioparticles was measured by live cell imagine using Incucyte. Results are summarized in FIG. 1. The degradation of PIP4K2C by Compound U in human dendritic cells enhanced their phagocytosis of bioparticles, emphasizing the intrinsic immunological benefits of PIP4K2C targeting in myeloid cells.

### Example B-5: Anti-Tumor Efficacy

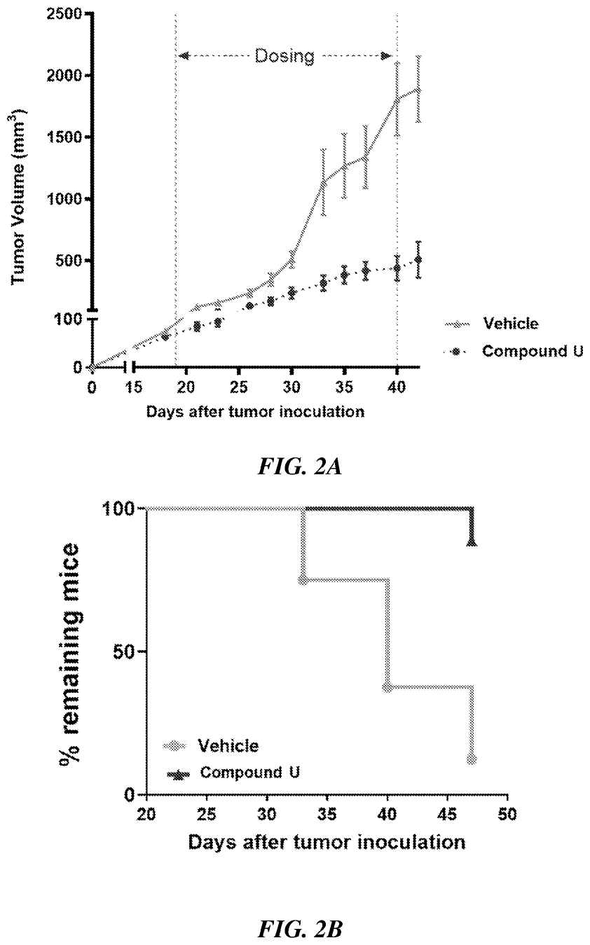

The ability of a PIP4K2C degrader to reduce tumor growth was assessed in the mouse syngeneic colorectal cancer model MC38-OVA. C57BL/6 female mice were implanted subcutaneously with 1×10⁶ MC38-OVA tumor cells. 20 days after tumor inoculation, the mice were randomized into groups of 10 animals and either Compound U or a vehicle control were administered every other day intraperitoneally at a 3 mg/kg dose for 3 weeks. Tumor volume was evaluated every other day by caliper. Results are summarized in FIG. 2A. Briefly, a marked reduction in tumor growth was observed in the treated group when compared to the vehicle control group. Consequently, the group dosed with 3 mg/kg of Compound U demonstrated a significant improvement in survival by end of study, as shown in FIG. 2B, demonstrating the beneficial impact of PIP4K2C degradation on cancer progression.

### Example B-6: Modulation of Phagocytosis

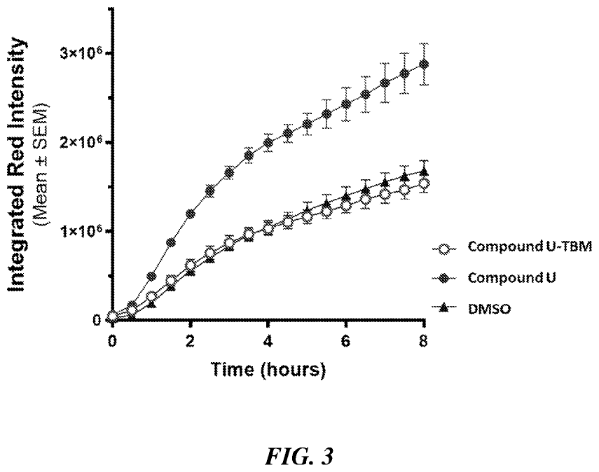

Primary human monocytes were isolated from PBMCs by negative bead selection and differentiated in vitro into immature dendritic cells in the presence of rhGM-CSF (800 u/ml) and rhIL-4 (400 U/mL) for 6 days, in the presence of Compound U or Compound U-TBM (50 nM) or DMSO. Compound U-TBM corresponds to the target binding moiety of Compound U, and it is an enzymatic inhibitor of PIP4K2C. The ability of dendritic cells to engulf pHrodo red bioparticles was measured by live cell imagine using Incucyte. The degradation of PIP4K2C by Compound U in human dendritic cells enhanced their phagocytosis of bioparticles, emphasizing the intrinsic immunological benefits of PIP4K2C targeting in myeloid cells. The same effect is not seen when using Compound U-TBM, demonstrating the unique impact of a degrader of PIP4K2C over an inhibitory compound alone. Results are summarized in FIG. 3.

### Example B-7: Impact on Tumor Antigen Uptake of PIP4K2C Deficiency in Malignant and Immune Cells

In order to evaluate the impact of PIP4K2C deficiency simultaneously in tumor cells and in immune cells, primary human monocytes were derived in vitro into immature dendritic cells in the presence of Compound U (50 nM) or DMSO. SW1417 CRC cells stably KO for PIP4K2C or NT control were labelled with pHrodo red dye and treated to die with H₂O₂ (2 mM) for 4 hrs. The dying tumor cells were harvested and co-cultured with the immature dendritic cells as indicated, and the uptake of the tumor material by the dendritic cells, as denoted by increased red fluorescence, was measured using an Incucyte live imager over 8 hours.

The absence of PIP4K2C in dendritic cells leads to an increased uptake of dying tumor-derived material, adding to the enhanced engulfment of PIP4K2C-deficient tumor cells by immune cells. This additive impact maximizes tumor cell clearance, antigen presentation and immune stimulation when targeting PIP4K2C in both tumor and immune cells. Results are summarized in FIG. 4.

### Example B-8: Degradation In Vivo in Rat and Dog PBMC

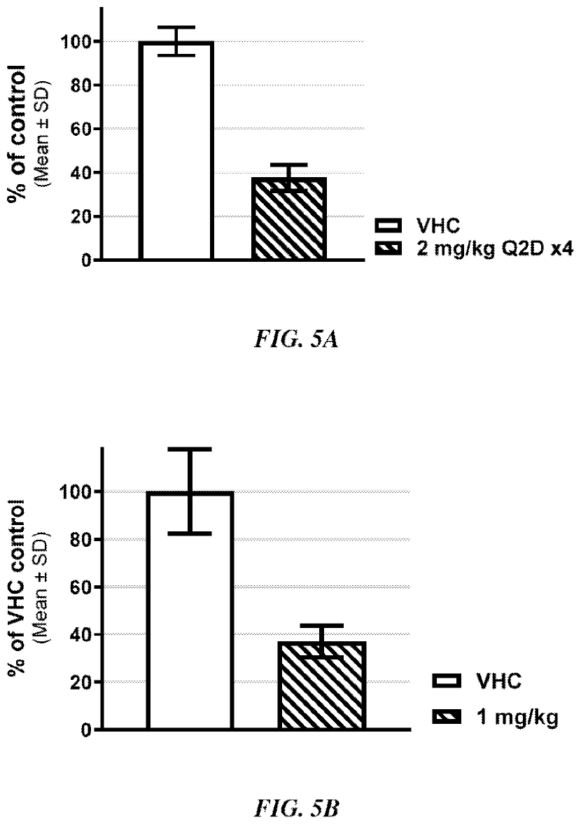

The ability of Compound U to degrade endogenous PIP4K2C in vivo across species was evaluated in rats and dogs. Briefly, animals were dosed IV with vehicle (VHC) or with Compound U at the indicated doses (1 mg/kg in dog, single dose, or 2 mg/kg in rat, Q2D, 4 doses). 24 hr after the last dose, blood was collected from each animal (3 animals per group), PBMCs were isolated and fixed, and the levels of PIP4K2C were assessed using intracellular flow cytometry using an AlexaFluor647-conjugated monoclonal anti-PIP4K2C antibody. Degradation was measured as percentage loss of PIP4K2C positive cells compared to vehicle control at the same time point. Results in rats are summarized in FIG. 5A, and results in dogs are summarized in FIG. 5B.

All references throughout, such as publications, patents, patent applications and published patent applications, are incorporated herein by reference in their entireties.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A compound of Formula (I′) 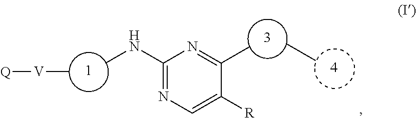
or a tautomer or stereoisomer thereof, or a pharmaceutically acceptable salt of any of the foregoing, wherein:
R is halo;
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Ring 3 is a monocyclic ring selected from the group consisting of 3- to 6-membered cycloalkyl optionally substituted with one or more R^{3a}; 4- to 6-membered heterocyclyl optionally substituted with one or more R^{3a}; phenyl optionally substituted with one or more R^{3b}; or 5- to 6-membered heteroaryl optionally substituted with one or more R^{3b};
Ring 1 is a 4- to 8-membered cycloalkyl or saturated 4- to 8-membered heterocyclyl, each of which is optionally substituted with one or more R¹;
V is a bond, —C₁₋₄alkylene-, —C(O)—, —C(O)O-#, —OC(O)#-C₁₋₄alkylene-C(O)-#, —C(O)N(R^{V})-#, —N(R^{V})C(O)-#, —OC(O)N(R^{V})-#, —N(R^{V})—C(O)O#, —C₁₋₄alkylene-C(O)N(R^{V})-# or —C₁₋₄alkylene-N(R^{V})C(O)-#, wherein # indicates the point of attachment to Ring 1; and
Q is R^{Q}, or Ring 5,
wherein Ring 5 is selected from the group consisting of 3- to 10-membered cycloalkyl optionally substituted with one or more R^{5a}, 4- to 12-membered heterocyclyl optionally substituted with one or more R^{5a}, and 5- to 12-membered heteroaryl optionally substituted with one or more R^{5b};
or
Q is of Formula (i) 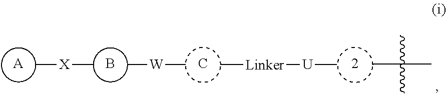
wherein:
Ring A is selected from the group consisting of 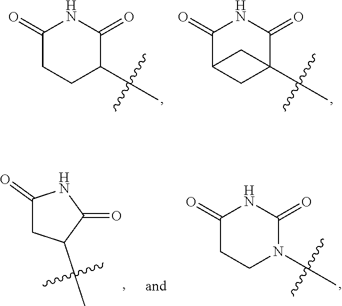 [SMILES: *C1CCC(=O)NC1=O ; *C12CC(C1)C(=O)NC2=O ; *C1CC(=O)NC1=O ; *N1CCC(=O)NC1=O]
each of which is optionally substituted with one or more C₁₋₄alkyl or halo;
X is a bond, —O—, —NH—, or —NHC(O)—;
Ring B is selected from the group consisting of 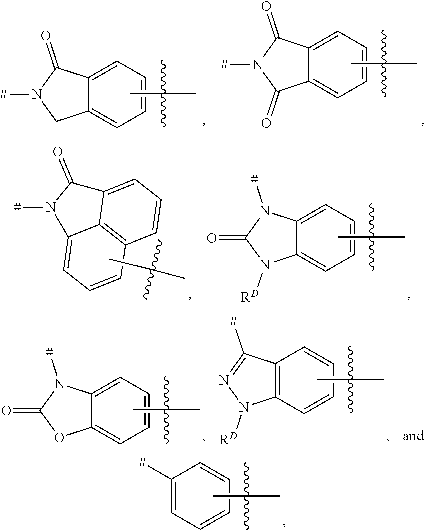
each of which is optionally substituted with one or more halo, C₁₋₄alkyl, C₁₋₄haloalkyl, or OH, and wherein R^{D} is H or C₁₋₄alkyl, wherein # indicates attachment to X;
W is a bond, —O—, —NH—, or —NHC(O)—;
Ring C is optional, and when present is 4- to 12-membered heterocyclyl optionally substituted with one or more R^{C};
Linker is a bond, —O—, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein
* indicates the point of attachment to Ring C when Ring C is present, and * indicates the point of attachment to W when Ring C is absent; and
** indicates the point of attachment to U when Ring 2 is present, and ** indicates the point of attachment to V when Ring 2 is absent;
L¹ is a bond or C₁₋₆ alkylene;
L² is a 3- to 10-membered cycloalkyl or 4- to 12-membered heterocyclyl optionally substituted with one or more R^{L}; and
L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y});
U is absent when Ring 2 is absent, and is a bond or C(O) when Ring 2 is present;
Ring 2 is optional, and when present is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b};
each R¹ is independently oxo, halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{2a}, R^{3a}, R^{4a}, and R^{5a} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{2b}, R^{3b}, R^{4b}, and R^{5b} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C₁₋₄hydroxyalkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R^{C} is independently halo, OH, C₁₋₄alkyl, or C₁₋₄haloalkyl;
each R^{L} is independently halo, OH, C₁₋₄alkyl, or C₁₋₄haloalkyl;
each R^{Q} is independently H, halo, OH, C₁₋₄alkyl, or C₁₋₄haloalkyl;
each R^{V} is independently H, halo, OH, C₁₋₄alkyl, or C₁₋₄haloalkyl;
each R^{w} is independently 3- to 10-membered cycloalkyl, phenyl optionally substituted with halo, or 4- to 12-membered heterocyclyl optionally substituted with hydroxyl, C₁₋₄hydroxyalkyl, or C₁₋₄aminoalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, 3- to 10-membered cycloalkyl, or C₁₋₄alkylene-S(O)ₙ—C₁₋₄alkyl; and
each n is independently 0, 1, or 2;
wherein:
(a) Ring 1 comprises an intraannular amide, or
(b) V comprises an amide or carbamate, or
(c) V is taken together with an atom of Ring 1, Ring 2, or Ring 5 to which it is attached, to form an amide or carbamate;
and wherein:
when Ring 3 is 5-membered heteroaryl, Ring 4 is present and is not cycloalkyl or tetrahydropyranyl; and
when Ring 1 is 8-membered heterocyclyl, Ring 3 is phenyl substituted with CN, Ring 4 is absent, Q is R^{Q}, and V is —OC(O)#, then R^{Q} is not tert-butyl.

2. The compound of claim 1, or a tautomer or stereoisomer thereof, or a pharmaceutically acceptable salt of any of the foregoing, wherein:
V is a bond, —C₁₋₄alkylene-, —C(O)—, —C₁₋₄alkylene-C(O)-#, —C(O)N(R^{V})-#, —N(R^{V})C(O)-#, —C₁₋₄ alkylene-C(O)N(R^{V})-# or —C₁₋₄alkylene-N(R^{V})C(O)-#, wherein # indicates the point of attachment to Ring 1;
Linker is a bond, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**,
each R^{2a}, R^{3a}, R^{4a}, and R^{5a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{2b}, R^{3b}, R^{4b}, and R^{5b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R^{V} is independently halo, OH, C₁₋₄alkyl, or C₁₋₄haloalkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, or C₁₋₄alkylene-S(O)ₙ—C₁₋₄alkyl; and
each n is independently 0, 1, or 2;
wherein:
(a) Ring 1 comprises an intraannular amide, or
(b) V comprises an amide, or
(c) V is taken together with an atom of Ring 1, Ring 2, or Ring 5 to which it is attached, to form an amide;
and wherein:
when Ring 3 is 5-membered heteroaryl, Ring 4 is present and is not cycloalkyl or tetrahydropyranyl; and
when Ring 1 is 8-membered heterocyclyl, Ring 3 is phenyl substituted with CN, Ring 4 is absent, Q is R^{Q}, and V is —OC(O)#, then R^{Q} is not tert-butyl.

3. The compound of claim 1 or 2, or a tautomer or stereoisomer thereof, or a pharmaceutically acceptable salt of any of the foregoing, wherein:
Ring 1 is a 4- to 8-membered cycloalkyl, saturated 4- to 8-membered heterocyclyl comprising at least one annular ether, saturated 4- to 8-membered heterocyclyl comprising at least one annular amine, or saturated 4- to 8-membered heterocyclyl comprising an annular amide, each of which is optionally substituted with one or more R¹;
and wherein:
(i) when Ring 1 is a 4- to 8-membered heterocyclyl ring comprising at least one annular amine, optionally substituted with one or more R¹, then V is attached to an annular nitrogen atom of Ring 1 and is —C(O)— or —C₁₋₄alkylene-C(O)-#, wherein # indicates the point of attachment to Ring 1;
(ii) when Ring 1 is a 4- to 8-membered heterocyclyl ring comprising at least one annular amide, optionally substituted with one or more R¹, then V is attached to an annular nitrogen atom of Ring 1 and is a bond or —C₁₋₄alkylene-; and
(iii) when Ring 1 is C₄₋₈ cycloalkyl or a 4- to 8-membered heterocyclyl ring comprising at least one annular ether and no annular amine or annular amide, each of which is optionally substituted with one or more R¹, then:
(a) V is —C(O)N(R^{V})-#, —N(R^{V})C(O)-#, —C₁₋₄alkylene-C(O)N(R^{V})-# or —C₁₋₄ alkylene-N(R^{V})C(O)-#, wherein # indicates the point of attachment to Ring 1; or
(b) Ring 5 is 4- to 12-membered heterocyclyl optionally substituted with one or more R^{5a}, and V is —C(O)— and is attached to an annular nitrogen atom of Ring 5; or
(c) Ring 2 is 4- to 12-membered heterocyclyl optionally substituted with one or more R^{2a}, and V is —C(O)— and is attached to an annular nitrogen atom of Ring 2.

4. The compound of any of claims 1-3, wherein R is chloro.

5. The compound of any of claims 1-3, wherein R is fluoro.

6. The compound of any of claims 1-5, wherein Ring 3 is 4- to 6-membered heterocyclyl optionally substituted with one or more R^{3a}.

7. The compound of claim 6, wherein Ring 3 is 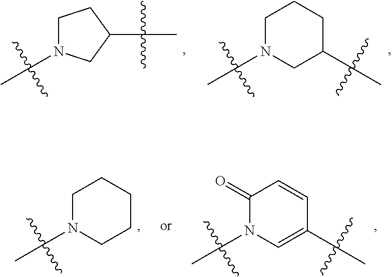 [SMILES: *C1CCN(*)C1 ; *C1CCCN(*)C1 ; *N1CCCCC1 ; *c1ccc(=O)n(*)c1]
each of which is optionally substituted with one or more R^{3a}.

8. The compound of any of claims 1-5, wherein Ring 3 is phenyl optionally substituted with one or more R^{3b}.

9. The compound of any of claims 1-5, wherein Ring 3 is 5- to 6-membered heteroaryl optionally substituted with one or more R^{3b}

10. The compound of claim 9, wherein Ring 3 is 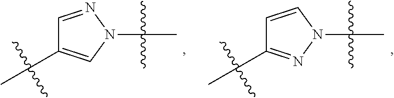 [SMILES: *c1cnn(*)c1 ; *c1ccn(*)n1]
each of which is optionally substituted with one or more R^{3b}.

11. The compound of any of claims 1-8, wherein Ring 4 is absent.

12. The compound of any of claims 1-8, wherein Ring 4 is 3- to 6-membered cycloalkyl.

13. The compound of claim 12, wherein Ring 4 is cyclobutyl, cyclopentyl, or cyclohexyl, optionally substituted with one or more R^{4a}.

14. The compound of any of claims 1-10, wherein Ring 4 is 4- to 6-membered heterocyclyl optionally substituted with one or more R^{4a}.

15. The compound of claim 14, wherein Ring 4 is 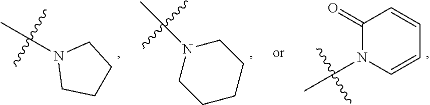 [SMILES: *N1CCCC1 ; *N1CCCCC1 ; *n1ccccc1=O]
each of which is optionally substituted with one or more R^{4a}.

16. The compound of any of claims 1-10, wherein Ring 4 is phenyl optionally substituted with one or more R^{4b}.

17. The compound of any of claims 1-10, wherein Ring 4 is 5- to 6-membered heteroaryl optionally substituted with one or more R^{4b}.

18. The compound of claim 17, wherein Ring 4 is 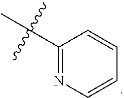 [SMILES: *c1ccccn1]
optionally substituted with one or more R^{4b}.

19. The compound of any of claims 1-5, wherein Ring 3 is phenyl optionally substituted by R^{3b}, and Ring 4 is phenyl optionally substituted by R^{4b}.

20. The compound of any of claims 1-5, wherein Ring 3 is phenyl optionally substituted by R^{3b}, or Ring 4 is phenyl optionally substituted by R^{4b}.

21. The compound of any of claims 1-5, wherein
Ring 3 is selected from the group consisting of 3- to 6-membered cycloalkyl optionally substituted with one or more R^{3a}, 4- to 6-membered heterocyclyl optionally substituted with one or more R^{3a}, and 5- to 6-membered heteroaryl optionally substituted with one or more R^{3b}; and
Ring 4 is selected from the group consisting of 3- to 6-membered cycloalkyl optionally substituted with one or more R^{4a}, 4- to 6-membered heterocyclyl optionally substituted with one or more R^{4a}, and 5- to 6-membered heteroaryl optionally substituted with one or more R^{4b}.

22. The compound of any of claims 1-10 or 14-21, wherein Ring 1 is 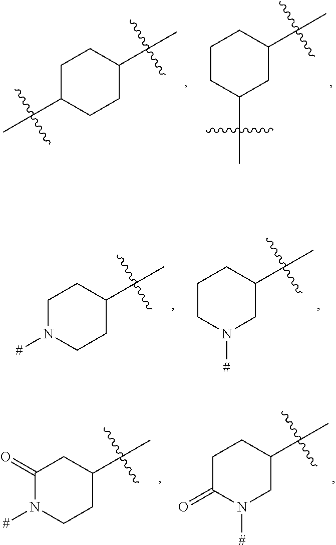
each of which is optionally substituted with one or more R¹, wherein # indicates attachment to V.

23. The compound of claim 22, wherein
Ring 1 is 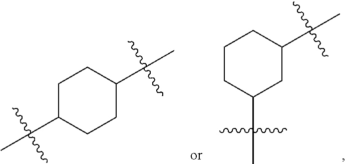 [SMILES: *C1CCC(*)CC1 ; *C1CCCC(*)C1]
each of which is optionally substituted with one or more R¹; and
V is —C(O)N(R^{V})—, —N(R^{V})C(O)—, —C₁₋₄alkylene-C(O)N(R^{V})-#, or —C₁₋₄alkylene-N(R^{V})C(O)-#, wherein ## indicates the point of attachment to Ring 1.

24. The compound of claim 22, wherein
Ring 1 is 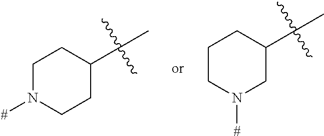
each of which is optionally substituted with one or more R¹, wherein # indicates attachment to V; and
V is —C(O)— or —C₁₋₄alkylene-C(O)-#, wherein # indicates attachment to Ring 1.

25. The compound of claim 22, wherein
Ring 1 is 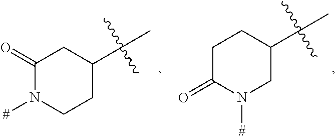
each of which is optionally substituted with one or more R¹, wherein # indicates attachment to V; and
V is bond or —C₁₋₄alkylene-.

26. The compound of any of claims 1-10 or 14-25, wherein Q is R^{Q}.

27. The compound of claim 26, wherein R^{Q} is H, methyl, or acetyl.

28. The compound of any of claims 1-10 or 14-25, wherein Q is Ring 5 and the compound is of Formula (II): 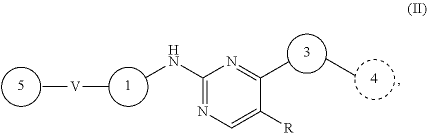
or a tautomer or stereoisomer thereof, or a pharmaceutically acceptable salt of any of the foregoing.

29. The compound of any of claims 1-10 or 14-25, wherein Q is of Formula (i) and the compound is of Formula (IV): 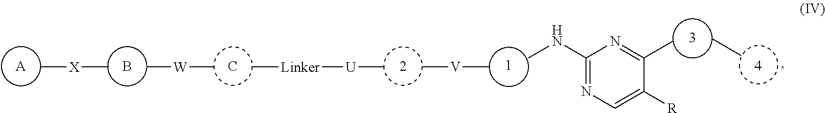
or a tautomer or stereoisomer thereof, or a pharmaceutically acceptable salt of any of the foregoing.

30. The compound of claim 29, wherein Ring A is 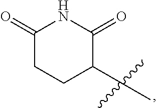 [SMILES: *C1CCC(=O)NC1=O]
optionally substituted with C₁₋₄alkyl or halo.

31. The compound of claim 29 or 30, wherein X is a bond or —NH—.

32. The compound of any of claims 29-31, wherein Ring B is 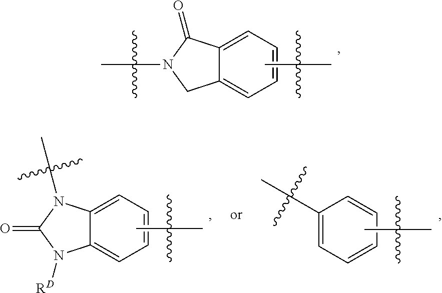
each of which is optionally substituted with one or more halo or OH, wherein R^{D} is H or C₁₋₄alkyl.

33. The compound of any of claims 29-32, wherein W is a bond or —O—.

34. The compound of any of claims 29-33, wherein Ring C is absent.

35. The compound of any of claims 29-33, wherein Ring C is 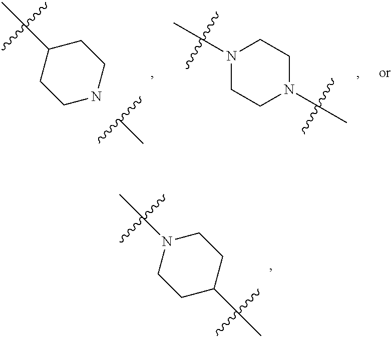
each of which is optionally substituted with one or more R^{C}.

36. The compound of any of claims 29-35, wherein Linker is a bond, C₁₋₁₀alkylene, or C(O)C₁₋₁₀alkylene.

37. The compound of any of claims 29-36, wherein Ring 2 is present and U is a bond.

38. The compound of any of claims 29-37, wherein Ring 2 is 4- to 12-membered heterocyclyl optionally substituted with one or more R^{2a}.

39. The compound of any of claims 1-23 or 26-38, wherein R^{V} is H.

40. The compound of any of claims 29-37 or 39, wherein Ring 2 is 3- to 10-membered cycloalkyl optionally substituted with one or more R^{2a}.

41. The compound of any of claims 29-39, wherein Ring 2 is 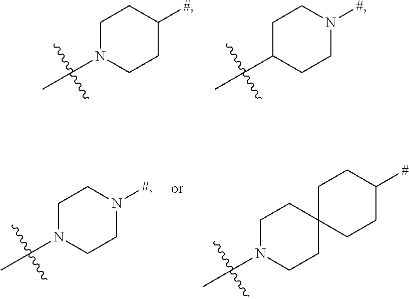
or each of which is optionally substituted with one or more R^{2a}, wherein # indicates attachment to V.

42. The compound of any of claims 29-39 or 41, wherein Ring 2 is 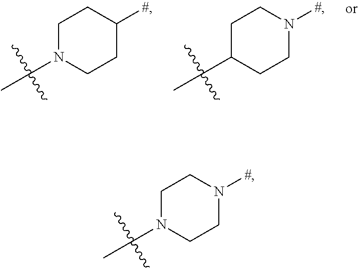
each of which is optionally substituted with one or more R^{2a}, wherein # indicates attachment to V.

43. The compound of claim 42, wherein Ring 2 is

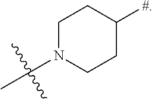

44. The compound of claim 43, wherein V is —C(O)— and Ring 1 is piperidinyl.

45. The compound of claim 41 or 42, wherein Ring 2 is 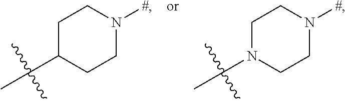
each of which is optionally substituted with one or more R^{2a}, wherein # indicates attachment to V.

46. The compound of claim 45, wherein V is —C(O)— and Ring 1 is cyclohexyl.

47. The compound of claim 45, wherein V is —C₁₋₄alkyl-C(O)— and Ring 1 is piperidinyl.

48. The compound of any of claims 27-35, wherein Ring 2, V, and Ring 1 are taken together to form:

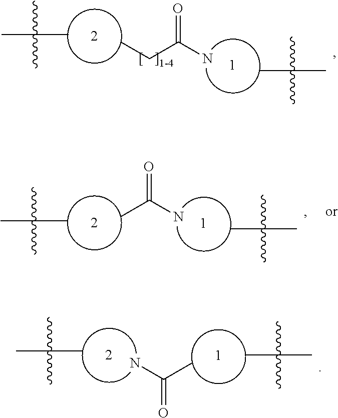

49. The compound of claim 48, wherein Ring 2, V, and Ring 1 are taken together to form:

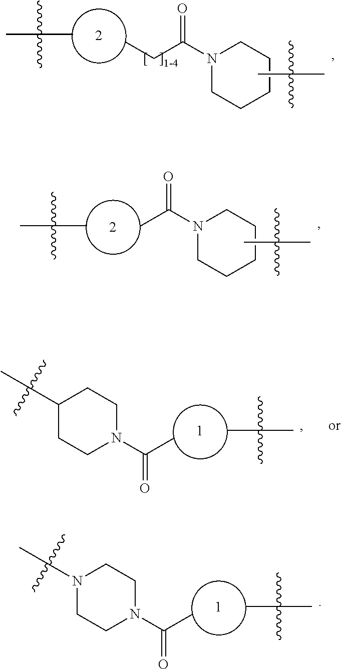

50. The compound of claim 40, wherein Ring 2 is cyclohexyl optionally substituted with one or more R^{2a}.

51. The compound of claim 50, wherein Ring 2 is 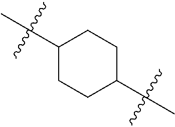 [SMILES: *C1CCC(*)CC1]
optionally substituted with one or more R^{2a}.

52. The compound of claim 50 or 51, wherein V is —C(O)N(R^{V})-#, wherein # indicates the point of attachment to Ring 1, and wherein Ring 1 is cyclohexyl.

53. The compound of any of claims 50-52, wherein Ring 2, V, and Ring 1 are taken together to form:

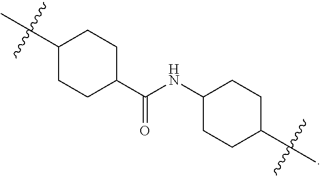

54. The compound of claim 1, wherein the compound is selected from the compounds provided in Table 1 and Table 2, a tautomer or stereoisomer thereof, or a pharmaceutically acceptable salt of any of the foregoing.

55. The compound of claim 54, wherein the compound is selected from the compounds provided in Table 1, a tautomer or stereoisomer thereof, or a pharmaceutically acceptable salt of any of the foregoing.

56. The compound of claim 54, wherein the compound is selected from the compounds provided in Table 2, a tautomer or stereoisomer thereof, or a pharmaceutically acceptable salt of any of the foregoing.

57. A pharmaceutical composition, comprising a compound of any of claims 1-56, a tautomer or stereoisomer thereof, or a pharmaceutically acceptable salt of any of the foregoing, and a pharmaceutically acceptable excipient.

58. The pharmaceutical composition of claim 57, wherein the compound is a compound of any of claims 26-28 or 55, a tautomer or stereoisomer thereof, or a pharmaceutically acceptable salt of any of the foregoing, and a pharmaceutically acceptable excipient.

59. The pharmaceutical composition of claim 57, wherein the compound is a compound of any of claims 29-53 or 56, a tautomer or stereoisomer thereof, or a pharmaceutically acceptable salt of any of the foregoing, and a pharmaceutically acceptable excipient.

60. A method of treating a disease or disorder associated with PIP4K2C, comprising administering to a subject a compound of any of claims 1-56, or a tautomer or stereoisomer thereof, or a pharmaceutically acceptable salt of any of the foregoing, or the pharmaceutical composition of claim 57.

61. A method of treating a disease or disorder by modulating the level or activity of PIP4K2C, comprising administering to a subject a compound of any of claims 1-56, or a tautomer or stereoisomer thereof, or a pharmaceutically acceptable salt of any of the foregoing, or the pharmaceutical composition of claim 57.

62. The method of claim 61, wherein the method modulates the activity of PIP4K2C, and wherein the method comprises administering to the subject a compound of any of claims 26-28 or 55, or a tautomer or stereoisomer thereof, or a pharmaceutically acceptable salt of any of the foregoing, or the pharmaceutical composition of claim 58.

63. The method of claim 61, wherein the method modulates the level of PIP4K2C, and wherein the method comprises administering to the subject a compound of any of claims 29-53 or 56, or a tautomer or stereoisomer thereof, or a pharmaceutically acceptable salt of any of the foregoing, or the pharmaceutical composition of claim 59.

64. The method of any one of claims 60-63, wherein the disease or disorder is a cancer, immune deficiency, autoimmune disease, infectious disease, or a combination thereof.

65. The method of claim 64, wherein the cancer is brain cancer, bladder cancer, breast cancer, triple negative breast cancer, cervical cancer, colorectal carcinoma, colorectal carcinoma with MSI, colorectal carcinoma with MSS, gastric cancer, head and neck squamous cell carcinoma, hepatocellular carcinoma, leukemia, lung cancer, small cell lung cancer, non-small cell lung cancer, lymphoma, melanoma, Merkel cell carcinoma, multiple myeloma, pancreatic ductal carcinoma, or renal cell carcinoma.

66. The method of claim 65, wherein the cancer is brain cancer, breast cancer, triple negative breast cancer, colorectal carcinoma, colorectal carcinoma with MSI, colorectal carcinoma with MSS, or non-small cell lung cancer.

67. The method of any of claims 60-63, wherein the disease or disorder is a neurodegenerative disease.

68. A method of treating a viral infection by modulating the level of PIP4K2C, comprising administering to a subject a compound of any of claims 29-53 or 56, or a tautomer or stereoisomer thereof, or a pharmaceutically acceptable salt of any of the foregoing, or the pharmaceutical composition of claim 59.

69. A method of treating necrotizing soft tissue infection (NSTI), comprising administering to a subject a compound of any of claims 1-56, or a tautomer or stereoisomer thereof, or a pharmaceutically acceptable salt of any of the foregoing, or the pharmaceutical composition of claim 57.

70. A method of modulating the level or activity of at least one of PIP4K2A, PIP4K2B, and PIP4K2C, comprising administering to a subject a compound of any of claims 1-56, or a tautomer or stereoisomer thereof, or a pharmaceutically acceptable salt of any of the foregoing, or the pharmaceutical composition of claim 57.

71. A compound of any of claims 1-56, or the pharmaceutical composition of claim 57, or a tautomer or stereoisomer thereof, or a pharmaceutically acceptable salt of any of the foregoing, for use in treating a disease or disorder associated with PIP4K2C in a subject in need thereof.

72. A compound of any of claims 1-56, or a tautomer or stereoisomer thereof, or a pharmaceutically acceptable salt of any of the foregoing, or the pharmaceutical composition of claim 57, for use in treating a disease or disorder by modulating the level or activity of PIP4K2C in a subject in need thereof.

73. The compound for use of claim 72, wherein the compound for use modulates the activity of PIP4K2C, and wherein the compound for use is a compound of any of claims 26-28 or 55, or a tautomer or stereoisomer thereof, or a pharmaceutically acceptable salt of any of the foregoing, or the pharmaceutical composition of claim 58.

74. The compound for use of claim 72, wherein the compound for use modulates the level of PIP4K2C, and wherein the compound for use is a compound of any of claims 29-53 or 56, or a tautomer or stereoisomer thereof, or a pharmaceutically acceptable salt of any of the foregoing, or the pharmaceutical composition of claim 59.

75. The compound for use of any one of claims 71-74, wherein the disease or disorder is a cancer, immune deficiency, autoimmune disease, infectious disease, or a combination thereof.

76. The compound for use of claim 75, wherein the cancer is brain cancer, bladder cancer, breast cancer, triple negative breast cancer, cervical cancer, colorectal carcinoma, colorectal carcinoma with MSI, colorectal carcinoma with MSS, gastric cancer, head and neck squamous cell carcinoma, hepatocellular carcinoma, leukemia, lung cancer, small cell lung cancer, non-small cell lung cancer, lymphoma, melanoma, Merkel cell carcinoma, multiple myeloma, pancreatic ductal carcinoma, or renal cell carcinoma.

77. The compound for use of claim 76, wherein the cancer is brain cancer, breast cancer, triple negative breast cancer, colorectal carcinoma, colorectal carcinoma with MSI, colorectal carcinoma with MSS, or non-small cell lung cancer.

78. The compound for use of any of claims 71-74, wherein the disease or disorder is a neurodegenerative disease.

79. A compound of any of claims 29-53 or 56, or a tautomer or stereoisomer thereof, or a pharmaceutically acceptable salt of any of the foregoing, or the pharmaceutical composition of claim 59, for use in treating a viral infection by modulating the level of PIP4K2C in a subject in need thereof.

80. A compound of any of claims 1-56, or a tautomer or stereoisomer thereof, or a pharmaceutically acceptable salt of any of the foregoing, or the pharmaceutical composition of claim 57, for use in treating necrotizing soft tissue infection (NSTI) in a subject in need thereof.

81. A compound of any of claims 1-56, or a tautomer or stereoisomer thereof, or a pharmaceutically acceptable salt of any of the foregoing, or the pharmaceutical composition of claim 57, for use in modulating the level or activity of at least one of PIP4K2A, PIP4K2B, and PIP4K2C in a subject in need thereof.

82. Use of a compound of any of claims 1-56, or a tautomer or stereoisomer thereof, or a pharmaceutically acceptable salt of any of the foregoing, for the manufacture of a medicament for treating a disease or disorder associated with PIP4K2C in a subject in need thereof.

83. Use of a compound of any of claims 1-56, or a tautomer or stereoisomer thereof, or a pharmaceutically acceptable salt of any of the foregoing, for the manufacture of a medicament for treating a disease or disorder by modulating the level or activity of PIP4K2C in a subject in need thereof.

84. The use of claim 83, wherein the use modulates the activity of PIP4K2C, and wherein the compound is a compound of any of claims 26-28 or 55, or a tautomer or stereoisomer thereof, or a pharmaceutically acceptable salt of any of the foregoing.

85. The use of claim 83, wherein the use modulates the level of PIP4K2C, and wherein the compound is a compound of any of claims 29-53 or 56, or a tautomer or stereoisomer thereof, or a pharmaceutically acceptable salt of any of the foregoing.

86. The use of any one of claims 82-85, wherein the disease or disorder is a cancer, immune deficiency, autoimmune disease, infectious disease, or a combination thereof.

87. The use of claim 86, wherein the cancer is brain cancer, bladder cancer, breast cancer, triple negative breast cancer, cervical cancer, colorectal carcinoma, colorectal carcinoma with MSI, colorectal carcinoma with MSS, gastric cancer, head and neck squamous cell carcinoma, hepatocellular carcinoma, leukemia, lung cancer, small cell lung cancer, non-small cell lung cancer, lymphoma, melanoma, Merkel cell carcinoma, multiple myeloma, pancreatic ductal carcinoma, or renal cell carcinoma.

88. The use of claim 87, wherein the cancer is brain cancer, breast cancer, triple negative breast cancer, colorectal carcinoma, colorectal carcinoma with MSI, colorectal carcinoma with MSS, or non-small cell lung cancer.

89. The use of any of claims 82-85, wherein the disease or disorder is a neurodegenerative disease.

90. Use of a compound of any of claims 29-53 or 56, or a tautomer or stereoisomer thereof, or a pharmaceutically acceptable salt of any of the foregoing, for the manufacture of a medicament for treating a viral infection by modulating the level of PIP4K2C in a subject in need thereof.

91. Use of a compound of any of claims 1-56, or a tautomer or stereoisomer thereof, or a pharmaceutically acceptable salt of any of the foregoing, for the manufacture of a medicament for treating necrotizing soft tissue infection (NSTI) in a subject in need thereof.

92. Use of a compound of any of claims 1-56, or a tautomer or stereoisomer thereof, or a pharmaceutically acceptable salt of any of the foregoing, for the manufacture of a medicament for modulating the level or activity of at least one of PIP4K2A, PIP4K2B, and PIP4K2C in a subject in need thereof.

93. A kit, comprising (i) a compound of any of claims 1-56, or a stereoisomer or tautomer thereof, or a pharmaceutically acceptable salt of any of the foregoing, or the pharmaceutical composition of claim 57, and (ii) instructions for use in treating an PIP4K-mediated disease, disorder, or condition in an individual in need thereof.

94. The method of claim 65 or 66, wherein the cancer is brain cancer.

95. The method of any of claims 65, 66, or 94 wherein the brain cancer is glioma or glioblastoma.

96. The method of any of claims 65, 66, or 94, wherein the brain cancer is brain stem glioma, gestational trophoblastic tumor glioma, cerebellar astrocytoma, cerebral astrocytoma/malignant glioma, ependymoma, medulloblastoma, supratentorial primitive neuroectodermal tumors, or visual pathway or hypothalamic glioma.

97. The method of claim 65 or 66, wherein the cancer is colorectal cancer.

98. The method of any of claims 65, 66, or 97, wherein the colorectal cancer is colorectal carcinoma, colorectal carcinoma with MSI, or colorectal carcinoma with MSS.

99. The method of any of claims 65, 66, or 97, wherein the colorectal cancer is associated with a hereditary syndrome.

100. The method of claim 65 or 66, wherein the cancer is non-small cell lung cancer.

101. The compound for use of claim 76 or 77, wherein the cancer is brain cancer.

102. The compound for use of any of claims 76, 77, or 101, wherein the brain cancer is glioma or glioblastoma.

103. The compound for use of any of claims 76, 77, or 101, wherein the brain cancer is brain stem glioma, gestational trophoblastic tumor glioma, cerebellar astrocytoma, cerebral astrocytoma/malignant glioma, ependymoma, medulloblastoma, supratentorial primitive neuroectodermal tumors, or visual pathway or hypothalamic glioma.

104. The compound for use of claim 76 or 77, wherein the cancer is colorectal cancer.

105. The compound for use of any of claims 76, 77, or 104, wherein the colorectal cancer is colorectal carcinoma, colorectal carcinoma with MSI, or colorectal carcinoma with MSS.

106. The compound for use of any of claims 76, 77, or 104, wherein the colorectal cancer is associated with a hereditary syndrome.

107. The compound for use of claim 76 or 77, wherein the cancer is non-small cell lung cancer.

108. The use of claim 87 or 88, wherein the cancer is brain cancer.

109. The use of any of claims 87, 88, or 108, wherein the brain cancer is glioma or glioblastoma.

110. The use of any of claims 87, 88, or 108, wherein the brain cancer is brain stem glioma, gestational trophoblastic tumor glioma, cerebellar astrocytoma, cerebral astrocytoma/malignant glioma, ependymoma, medulloblastoma, supratentorial primitive neuroectodermal tumors, or visual pathway or hypothalamic glioma.

111. The use of claim 87 or 88, wherein the cancer is colorectal cancer.

112. The use of claim 87, 88, or 111, wherein the colorectal cancer is colorectal carcinoma, colorectal carcinoma with MSI, or colorectal carcinoma with MSS.

113. The use of claim 87, 88, or 111, wherein the colorectal cancer is associated with a hereditary syndrome.

114. The use of claim 87 or 88, wherein the cancer is non-small cell lung cancer.

115. The method of any one of claims 60-63, wherein the disease or disorder is related to loss-of-function mutations of the phosphoinositide phosphatase FIG. 4.

116. The method of claim 115, wherein the disease or disorder is Charcot-Marie-Tooth.

117. The method of claim 115 or 116, wherein the disease or disorder is Charcot-Marie-Tooth Type 4J.

118. The compound for use of any one of claims 71-74, wherein the disease or disorder is related to loss-of-function mutations of the phosphoinositide phosphatase FIG. 4.

119. The compound for use of claim 118, wherein the disease or disorder is Charcot-Marie-Tooth.

120. The compound for use of claim 118 or 119, wherein the disease or disorder is Charcot-Marie-Tooth Type 4J.

121. The use of any one of claims 82-85, wherein the disease or disorder is related to loss-of-function mutations of the phosphoinositide phosphatase FIG. 4.

122. The use of claim 121, wherein the disease or disorder is Charcot-Marie-Tooth.

123. The use of claim 121 or 122, wherein the disease or disorder is Charcot-Marie-Tooth Type 4J.

124. The method of claim 68, wherein the compound, or a tautomer or stereoisomer thereof, or a pharmaceutically acceptable salt of any of the foregoing, is administered in combination with a vaccine for viral infections to enhance the immune response.

125. The compound for use of claim 79, wherein the compound, or a tautomer or stereoisomer thereof, or a pharmaceutically acceptable salt of any of the foregoing, is administered in combination with a vaccine for viral infections to enhance the immune response.

126. The use of claim 90, wherein the medicament is administered in combination with a vaccine for viral infections to enhance the immune response.
